(19) United States
(12) Patent Application Publication
(10) Pub. No.: US 2006/0046253 A1
(43) Pub. Date: Mar. 2, 2006

(54) Method for analyzing genes of industrial yeasts

(75) Inventors: Yoshihiro Nakao, Kyoto (JP); Norihisa Nakamura, Kyoto (JP); Yukiko Kodama, Osaka (JP); Tomoko Fujimura, Osaka (JP); Toshihiko Ashikari, Osaka (JP)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: SUNTORY LIMITED, Osaka-shi (JP)

(21) Appl. No.: 10/932,182

(22) Filed: Sep. 2, 2004

(51) Int. Cl. C12Q 1/68; C12N 1/18; C12N 15/74

(52) U.S. Cl. 435/6; 435/483; 435/254.21

(57) ABSTRACT

An object of the present invention is to provide a method for analyzing gene of an industrial yeast. The method of the present invention comprises (a) determining the whole genome sequence of the industrial yeast; and
(c-1) selecting a gene of the industrial yeast encoding an amino acid sequence having 70 to 97% identity to an amino acid sequence encoded by the gene of Saccharomyces cerevisiae, or
(c-2) selecting a gene of the industrial yeast consisting of a nucleotide sequence having 60 to 94% identity to the nucleotide sequence of the gene of Saccharomyces cerevisiae.

### SEQUENCE LISTING

The Sequence Listing is submitted on one compact disc including a duplicate. The material on the compact disc including the duplicate copy is hereby incorporated by reference in its entity. The compact disc including the duplicate copy contains a file named “YCT966 sequence listing”, which was created on Aug. 23, 2004 (and stored on the compact disc on Sep. 2, 2004) and is 52,018 kilobytes.

Summary of the sequence listing is as follows:

SEQ ID NO: 1 represents the nucleic acid sequence for the nonSc-SSU1 gene.

SEQ ID NO: 2 represents the nucleic acid sequence for the nonSc-MET14 gene.

SEQ ID NO: 3 represents the amino acid sequence for the nonSc-SSU1 protein deduced from the nucleic acid sequence.

SEQ ID NO: 4 represents the amino acid sequence for the nonSc-MET14 protein deduced from the nucleic acid sequence.

SEQ ID NOs: 5-32 represent oligonucleotide primers for amplification.

SEQ ID NOs: 33-6236 represent 6204 DNA sequences of non-Sc type ORFs, which can be preferably used as probes of the microarry.

SEQ ID NOs: 6237-75336 represent sequences of oligonucleotides based on DNA sequences of SEQ ID NOs: 33-6236, which can be preferably used as probes of the microarry.

SEQ ID NOs: 75337-82784 represent 7448 DNA sequences of Sc type ORFs.

SEQ ID NOs: 82785-166153 represent sequences of oligonucleotides based on DNA sequences of SEQ ID NOs: 75337-82784.

SEQ ID NOs: 166154-166181 represent 28 DNA sequences of mtochondrial ORFs from 34/70 strain, which can be preferably used as probes of the microarry.

SEQ ID NOs: 166182-166489 represent sequences of oligonucleotides based on DNA sequences of SEQ ID NOs: 166154-166181, which can be preferably used as probes of the microarry.

SEQ ID NOs: 166490-167042 represent 553 DNA sequences which have not been identified as the above non-Sc ORFs but have significant similarity to the proteins of S. cerevisiae using NCBI-BlastX homology searching.

SEQ ID NOs. 167043-173124 represent sequences of oligonucleotides based on DNA sequences of SEQ ID NOs: 166490-167042.

SEQ ID NOs: 173125-174603 represent 1479 DNA sequences ORFs which have not been identified as the above, but have significant similarity to the proteins of S. cerevisiae using NCBI-BlastP homology searching.

SEQ ID NOs. 174604-190810 represent sequences of oligonucleotides based on DNA sequences of SEQ ID NOs: 173125-174603.

SEQ ID NOs: 190811-191213 represent 403 S. cerevisiae ORFs from SGD which are not identified as Sc type ORFs in strain 34/70.

SEQ ID NOs: 191214-195587 represent sequences of oligonucleotides based on DNA sequences of SEQ ID NOs: 190811-191213.

SEQ ID NOs: 195588-195651 represent 64 DNA sequences of genes from Saccharomyces cerevisiae, Bacillus subtulis, Escherichia coli., or Bacteriophage P1, used as internal standard.

SEQ ID NOs: 195652-197021 represent sequences of oligonucleotides based on DNA sequences of SEQ ID NOs: 195588-195651.

The character “n” in various nucleotides sequences of the Sequence Listing is used in accordance with its well known meaning in the art to mean or represent a or g or c or t/u, unknown, or other.

### TECHNICAL FIELD

The present invention relates to a method analyzing genes of an industrial yeast used for the production of an alcoholic beverage such as beer or sake, a fuel alcohol, etc. and particularly for genes of brewing yeast used for the production of an alcoholic beverage. More particularly, it relates to a method where, in the production of an alcoholic beverage, DNA sequence information of brewing yeast is compiled in a database so that the gene which participates in increase in productivity and/or improvement in flavor such as stabilization, reinforcement, etc. of the flavor is selected; a method for breeding yeast suitable for the brewing in which expression of a gene is controlled, such as yeast in which the selected gene is disrupted or yeast in which the gene is overexpressed; and a method for the production of an alcoholic beverage using the bred yeast.

### BACKGROUND ART

Development of techniques for production of fuel alcohols, alcoholic beverages such as beer or sake, etc. has been carried out using industrial yeast. Especially in the production of an alcoholic beverage using brewing yeast, there has seen a brisk development in the techniques for increasing productivity and improving flavor such as stabilization or enhancement of flavor of an alcoholic beverage.

The most consumed alcoholic beverage in the world is beer and the amount of beer produced in the world in 2001 was about 140,000,000 kL. Type of beer is roughly classified into three depending upon type of yeast and fermentation method. The three types are, naturally fermented beer where fermentation is carried out utilizing yeast and microorganisms inhabiting in breweries; ale-type beer where fermentation is carried out using a top fermenting yeast belonging to Saccharomyces cerevisiae (hereinafter, abbreviated as S. cerevisiae) at the temperature of 20 to 25° C. and the following aging period is shortened; and lager-type beer where fermentation is carried out using a bottom fermenting yeast belonging to Saccharomyces pastorianus at the temperature of 6 to 15° C. and then subjected to a low-temperature aging. At present, not less than 90% of the beer produced in the world is a lager-type beer and, therefore, the bottom fermenting yeast that is used for brewing of the lager-type beer has been most widely used in beer brewing.

In the so-called fermentation production where production is carried out using a microorganism including the above-mentioned brewing yeast, it is important that the fermentation process is optimized and that the useful strain is selected and bred, in order to increase productivity and improve quality of the product.

In the case of optimization of beer brewing, there has been conducted a method where an amount of yeast metabolites such as alcohol (e.g. ethanol), ester, organic acid, etc. are monitored, and then temperature, quantity of airflow, content of raw material, etc. are controlled. In such a case, material uptake and excretion by yeast cells and metabolism in the cells are handled as a black box and only very superficial control is carried out. In addition, for the purpose of giving, for example, high flavor to an alcoholic beverage, a process control method for suppressing the amount of oxygen supply during beer brewing or the like has been tried. In such a method, however, growth rate of the yeast itself is reduced due to insufficient oxygen, and adverse effect such as retardation of fermentation and/or deterioration of beer quality may arise. Accordingly, there has been a limit on the improvement in productivity and quality of beer by means of optimization of fermentation processes.

On the other hand, with regard to a method of breeding useful industrial yeast such as useful beer yeast, a technique for selecting desirable strain has widely been used rather than actual breeding. Beer brewing per se has been performed since well before the discovery of microorganisms by Pasteur. In the beer brewing, a method of selecting more suitable strain of beer yeast from many strains of yeast used in the beer brewery has been traditionally carried out while there have been few cases where beer yeast with good traits is positively bred.

As an example of a positive breeding method, there is a method where artificial mutagenesis by chemicals or radioactive rays is used. However, brewing yeast, particularly a bottom fermenting yeast which is widely used in beer brewing, is in many cases a polyploid. In that case, it is not possible to produce the desired mutant unless mutation takes place in all of the alleles to be mutated. Accordingly, although it is possible to induce desirable mutation in the case of a haploid laboratory yeast, it is substantially impossible in the case of beer yeast which is a polyploid.

In recent years, there has been tried a breeding where mutation or cross-breeding is carried out by using spores isolated from bottom fermenting yeast (c.f., for example, Non-Patent Document 1). However, the bottom fermenting yeast is a polyploid, and has complicated chromosome structure. Therefore, isolation of spores having proliferation ability is difficult, and moreover, it is almost impossible to obtain a strain with good traits from them.

On the other hand, it has recently become possible that desired genes are introduced and expressed in the brewing yeast using a genetic engineering technique, whereby it has become possible to breed yeast with the desired character by using the results of functional analysis of genes and the genes which have been functionally analyzed. However, as compared with the baker's yeast (S. cerevisiae; c.f., for example, Non-Patent Document 2) of which the whole genome sequence is already clarified, the whole genome sequence of the bottom fermenting yeast has not been clarified and there have been only a very few findings about the gene participating in brewing character specific to bottom fermenting yeast and about the function of the said gene in beer brewing.

In recent years, transcriptome analysis has been conducted using DNA array where DNA fragments or nucleotide oligomers, each of which has a partial sequence of structural gene or internal region of the chromosome are fixed on solid support. For example, Olesen, et al. conducted a comprehensive genetic expression analysis of bottom fermenting yeast during the brewing using GeneFilters (manufactured by Research Genetics Co.) (c.f., for example, Non-Patent Document 3). However, since the whole genome sequence of bottom fermenting yeast has not been clarified yet, it is ambiguous that what gene is monitored for its expression precisely. As a result, such information is quite insufficient to apply to metabolic analysis of bottom fermenting yeast, and to breeding of useful yeast, and to control of beer brewing process.

At present, the whole genome sequences of more than 100 species of microorganisms have been determined (c.f., for example, Non-Patent Document 6) including S. cerevisiae, Escherichia coli (c.f., for example, Non-Patent Document 4) and Mycobacterium tuberculosis (c.f., for example, Non-Patent Document 5). On the basis of the determined DNA sequences, genes of these microorganisms are identified and function of an enormous number of genes have been predicted without conducting genetic, biochemical and molecular biological experiments. However, industrial yeast such as brewing yeast which has high ploidy and complicated chromosome structure, and thus an assembly (an operation for connecting the DNA sequences) is presumed to be difficult. Therefore, the whole genome sequence of bottom fermenting yeast which contains two different types of genome (c.f., for example, Non-Patent Document 7) has not been reported yet.

In the production of specific alcohols or alcoholic beverages, there is a technique to increase concentration of sulfite in the product for control of flavor. Sulfite is known as a compound which has anti-oxidative activity, and has been widely used as an antioxidant in the fields of food, beverage and pharmaceuticals, and also in an alcoholic beverage. For example, in the case of wine that requires a long aging period, sulfite plays an important role for the preservation of its quality. It is also known that, in beer brewing, the quality preservation period becomes long in accordance with the increase in concentration of sulfite contained in the product. Thus, when the amount of sulfite in the product is increased, it is possible to prepare a product that has excellent flavor stability and a long quality preservation period.

The simplest way to increase the amount of sulfite in the product is addition of sulfite. In Japan, so far as wine is concerned, it is permitted by the Ministry of Health, Labor and Welfare to add sulfite to an extent of not more than 350 ppm in terms of residual sulfite concentration. In that case, however, since sulfite is categorized as food additives, it is not appropriate to add sulfite to beer when a negative image of consumers to food additives is taken into consideration.

However, the yeast used in brewing produces hydrogen sulfide by the reduction of sulfate in the medium in order to synthesize sulfur-containing metabolites such as sulfur-containing amino acids. Sulfite is an intermediate metabolite of this pathway. If sulfite is efficiently excreted outside the cells during fermentation period, the amount of sulfite both in the wort and in the product can be increased.

There are two methods for increasing sulfite concentration in the wort during fermentation. One is control of fermentation process and another is breeding of brewing yeast. As for control of fermentation process, amount of sulfite produced during fermentation is inversely proportional to the concentration of dissolved oxygen and, therefore, there has attempted, a method where the concentration of dissolved oxygen is reduced so that amount of sulfite is increased and at the same time the oxidation of sulfite is suppressed. However, in that method, growth rate of yeast is reduced due to lack of oxygen, which has negative effects such as retardation of fermentation and deterioration of quality. Therefore that method is not practical.

On the other hand, as mentioned above, a genetic engineering technique has been developed for breeding brewing yeast. For example, there are some reports focused on sulfur metabolism of yeast. Sulfite (SO₂) is an intermediate product of sulfur-containing amino acid and vitamin synthesis and is produced via a pathway of sulfate ion (SO₄²⁻)→APS (adenyl sulfate)→PAPS (phosphoadenylyl sulfate)→sulfite ion (SO₃²⁻) where the sulfate ion is incorporated from outside of the cells. There is an attempt that copy numbers of MET 3 gene participating in the reaction of sulfate ion (SO₄²⁻)→APS (adenylyl sulfate) and of MET 14 gene participating in the reaction of APS (adenylyl sulfate)→PAPS (phosphoadenylyl sulfate) are increased to improve the ability of the yeast for the production of sulfite (c.f., for example, Non-Patent Document 8). There is another example of an attempt where reduction of sulfite ion (SO₃²⁻) is inhibited by disruption of MET 10 gene whereby amount of sulfite produced by the yeast is increased (c.f., for example, Non-Patent Document 9). According to such attempts, amount of sulfite produced by an MET 10 gene disruptant is increased to an extent of not less than ten-fold of the parental strain, but on the other hands some retardation in fermentation and increase in the amounts of acetaldehyde and 1-propanol in the beer are noted, which has become a problem for the practical use.

Accordingly, although development of breeding methods for industrial yeast such as brewing yeast using genetic engineering have been in progress, it is the current status that, due to insufficient genomic information of brewing yeast, selection of the gene participating in a brewing character of brewing yeast, analysis of function of protein encoded by the gene and utilization of those findings for breeding have not been sufficiently carried out.

Thus, a method for breeding yeast which shows the desired character without deterioration of fermentation speed and product quality has not been established yet and there has been a big demand for the development of such a method not only in the brewing industry but also in the industries where yeast is used.

(Non-Patent Document 1) C. Gjermansen: “Construction of a hybrid brewing strain of Saccharomyces carlsbergensis by mating of meiotic segregants”, Carlsberg Res. Commun., volume 46, pages 1 to 11 (1981).

(Non-Patent Document 2) A. Goffeau, et al.: “The Yeast Genome Directory”, Nature, volume 387, pages 5 to 105 (1997).

(Non-Patent Document 3) K. Olesen, et al.: “The dynamics of the Saccharomyces carlsbergensis brewing yeast transcriptome during a production-scale lager beer fermentation”, FEMS Yeast Research, volume 2, pages 563 to 573 (2000).

(Non-Patent Document 4) F. R. Blattner, et al.: “The Complete Genome Sequence of Escherichia coli K-12”, Science, volume 277, pages 1453-1462 (1997).

(Non-Patent Document 5) S. T. Cole, et al.; “Deciphering the biology of Mycobacterium tuberculosis from the complete genome sequence”, Nature, volume 393, pages 537-544 (1998).

(Non-Patent Document 6) The National Center for Biotechnology Information, http://www.ncbi.nlm.nih.gov/PMGifs/Genomes/micr.html.

(Non-Patent Document 7) Y. Tamai et al.:“Co-existence of two types of chromosome in the fermenting yeast, Sacchaomyces cerevisiae”, Yeast, volume 10, pages 923-933 (1998).

(Non-Patent Document 8) C. Korch, et al.: Proc. Eur. Brew. Conv. Congress, Lisbon, pages 201-208 (1991).

(Non-Patent Document 9) J. Hansen, et al.: “Inactivation of MET 10 in brewer's yeast specifically increases SO₂ formation during beer production”, Nature Biotech., volume 14, pages 1587-1591 (1996).

(Non-Patent Document 10) T. Sijen, et al.: “Transcriptional and posttranscriptional gene silencing are mechanistically related”, Curr. Biol., volume 11, pages 436-440 (2001)

(Non-Patent Document 11) N. Goto, et al.: “SSU1-R, a sulphite resistance gene of wine yeast, is an allele of SSU 1 with a different upstream sequence”, J. Ferment. Bioeng., volume 86, pages 427-433 (1998).

(Non-Patent Document 12) D. Avram, et al.: “SSU 1 encodes a plasma membrane protein with a central role in a network of proteins conferring sulfite tolerance in Saccharomyces cerevisiae”, J. Bacteriol., volume 179, pages 5971-5974 (1997).

(Non-Patent Document 13) H. Park, et al.; “SSU 1 mediates sulphite efflux in Saccharomyces cerevisiae”, Yeast, volume 16, pages 881-888 (2000).

### DISCLOSURE OF THE INVENTION

An object of the present invention is to provide a method of analyzing and selecting genes participating in the desired brewing character, which is achieved in such a manner that a database compiling the whole genome sequence (hereinafter, may be abbreviated as genomic DB) of industrial yeast, particularly brewing yeast used for an alcoholic beverage such as beer, is prepared; gene that the brewing yeast possesses is selected from the database; functional analysis of the gene may be carried out by disruption or overexpression. Another object of the present invention is to provide a DNA array which is useful for analyzing method of genes of an industrial yeast. Further object is to provide a breeding method of the yeast showing the brewing character which the said gene participates in and also a method of producing an alcohol or an alcoholic beverage where productivity and quality are improved using the said yeast. Still another object is to provide genes mentioned above and peptides encoded by the said genes.

It has been known that brewing yeast widely used for industrial purpose is a polyploid and especially, bottom fermenting yeast is an allopolyploid which is composed of at least two kinds of genomes. One of the genomes is thought to be a genome derived from S. cerevisiae of which the whole genome sequence has been clarified, while the source of another genome(s) has not been clarified yet.

The present inventors have determined the whole genome sequence of the bottom fermenting yeast in order to find unidentified genes displaying essential functions for excellent brewing. The amino acid sequences of the bottom fermenting yeast were then compared with those registered in the genomic DB for S. cerevisiae, and functions of proteins encoded by genes of the brewing yeast were estimated. As a result, it has been clarified that the genes of the bottom fermenting yeast are roughly classified into Sc type genes showing nearly 100% amino acid identity to S. cerevisiae and non-Sc type genes showing around 70 to 97% amino acid identify (this corresponds to around 60 to 94% identity in nucleotide level). Moreover, it has been clarified that the bottom fermenting yeast has complicated chromosome structure consists of Sc-type chromosomes, non-Sc-type chromosomes and Sc/non-Sc-type chimera chromosomes. Structure of the whole chromosomes of the bottom fermenting yeast is shown in FIG. 1. On the basis of genomic information clarified by the present invention, the present inventors have found such an unexpectedly complicated structure of chromosomes, and developed a screening method for the genes of bottom fermenting yeast.

To be more specific, there has been achieved a method for analyzing gene of an industrial yeast comprising

(a) determining nucleotide sequence of the whole genome of the industrial yeast, particularly bottom fermenting yeast which is one of the brewing yeasts,; and

(c-1) selecting a gene of the industrial yeast encoding an amino acid sequence having 70 to 97% identity to an amino acid sequence encoded by the gene of Saccharomyces cerevisiae, or

(c-2) selecting a gene of the industrial yeast consisting of a nucleotide sequence having 60 to 94% identity to the nucleotide sequence of the gene of Saccharomyces cerevisiae.

Preferably, the method of the present invention comprises,

(b) comparing the genome sequence with the whole genome sequence of Saccharomyces cerevisiae after the step (a) and before the step (c-1) or (c-2).

Preferably, the analyzing method of the present invention comprises, (d) carrying out functional analysis of the selected gene, after the step (c-1) or (c-2), whereby the brewing character given to the yeast by the genes are identified. The present inventors have repeatedly carried out intensive investigations on the basis of those findings and accomplished the present invention.

Thus, the present invention includes the following embodiments:

1. A method for analyzing gene of an industrial yeast comprising

(a) determining nucleotide sequence of the whole genome sequence of the industrial yeast; and

(c-1) selecting a gene of the industrial yeast encoding an amino acid sequence having 70 to 97% identity to an amino acid sequence encoded by the gene of Saccharomyces cerevisiae, or

(c-2) selecting a gene of the industrial yeast consisting of a nucleotide sequence having 60 to 94% identity to the nucleotide sequence of the gene of Saccharomyces cerevisiae.

2. The analyzing method according to Embodiment 1, comprising,

(b) comparing the genome sequence with the whole genome sequence of Saccharomyces cerevisiae after the step (a) and before the step (c-1) or (c-2).

3. The analyzing method according to Embodiment 1 or 2, comprising

(d) carrying out functional analysis of the selected gene after the step (c-1) or (c-2).

4. The analyzing method according to Embodiment 3, which comprises screening genes participating in increase in productivity and/or improvement in flavor in the production of an alcohol or an alcoholic beverage by the functional analysis of the step (d).

5. The analyzing method according to Embodiment 3 or 4, wherein functional analysis is, performed by disruption of a gene or overexpression of a gene.

6. The analyzing method according to any of Embodiments 1-5, wherein the industrial yeast is a brewing yeast.

7. The analyzing method according to Embodiment 6, wherein the brewing yeast is a beer yeast.

8. The analyzing method according to Embodiment 7, wherein the beer yeast is a bottom fermenting yeast.

9. A gene of the industrial yeast encoding an amino acid sequence having 70 to 97% identity to an amino acid sequence encoded by the gene of Saccharomyces cerevisiae, or consisting of a nucleotide sequence having 60 to 94% identity to the nucleotide sequence of the gene of Saccharomyces cerevisiae, which is obtained by the analyzing method according to any one of Embodiments 1-8.

10. A gene library comprising one or more of genes of the industrial yeast each of which encodes an amino acid sequence having 70 to 97% identity to an amino acid sequence encoded by the gene of Saccharomyces cerevisiae, or consists of a nucleotide sequence having 60 to 94% identity to the nucleotide sequence of the gene of Saccharomyces cerevisiae, which is obtained by the analyzing method according to any one of Embodiments 1-8.

11. The gene library according to Embodiment 10, wherein the gene of the industrial yeast is comprised in, consists of or has any nucleotide sequence selected from SEQ ID NOs:33 to 6236, SEQ ID NOs:166154 to 166181, SEQ ID NOs:166490 to 167042 and SEQ ID NOs:173125 to 174603.

12. A DNA array comprising one or more of DNAs, wherein each DNA is selected from at least one group of (1) to (4):

(1) DNA consisting of a nucleotide sequence of an open reading frame of the genome sequence of an industrial yeast which encodes an amino acid sequence having 70 to 97% identity to an amino acid sequence encoded by the gene of Saccharomyces cerevisiae, or a nucleotide sequence complementary to the above nucleotide sequence, or a nucleotide sequence of continuous 10 or more nucleotides selected from the above nucleotide sequences;

(2) DNA consisting of a nucleotide sequence of the genome sequence of an industrial yeast other than from open reading frames which consists of a nucleotide sequence having 60 to 94% identity to the nucleotide sequence of the gene of Saccharomyces cerevisiae, or a nucleotide sequence complementary to the above nucleotide sequence, or a nucleotide sequence of continuous 10 or more nucleotides selected from the above nucleotide sequences;

(3) DNA having a nucleotide sequence of any of SEQ ID NOs:166490 to 167042, or DNA having a nucleotide sequence of any of SEQ ID NOs:167043 to 173124; and

(4) DNA having a nucleotide sequence of any of SEQ ID NOs:173125 to 174603, or DNA having a nucleotide sequence of any of SEQ ID NOs:174604 to 190810.

13. The DNA array according to Embodiment 12 comprising DNAs wherein at least one DNA is selected from every group of (1) to (4).

14. The DNA array according to Embodiment 12 or 13, wherein the DNAs of (1) is comprised in, consists of or has any nucleotide sequence selected from SEQ ID NOs:33 to 6236 and SEQ ID NOs:166154 to 166181, and SEQ ID NOs:6237 to 75336 and SEQ ID NOs:166182 to 166489.

15. The DNA array according to any one of Embodiments 12-14, which further comprises, in addition to at least one group of DNAs selected from (1) to (4), DNAs of the following (5) and/or (6),:

(5) DNA consisting of a nucleotide sequence of an open reading frame of the genome sequence of an industrial yeast which encodes an amino acid sequence having identity of more than 97% to an amino acid sequence encoded by the gene of Saccharomyces cerevisiae, or a nucleotide sequence complementary to the above nucleotide sequence, or a nucleotide sequence of continuous 10 or more nucleotides selected from the above nucleotide sequences; and

(6) DNA consisting of a nucleotide sequence of the genome sequence of an industrial yeast other than from open reading frames which consists of a nucleotide sequence having identity of more than 94% to the nucleotide sequence of the gene of Saccharomyces cerevisiae, or a nucleotide sequence complementary to the above nucleotide sequence, or a nucleotide sequence of continuous 10 or more nucleotides selected from the above nucleotide sequences, and optionally DNAs of the following (5′) and/or (6′):

(5′) DNA consisting of a nucleotide sequence which contains mismatch of 1 or more base(s) to the DNA of (5), or a nucleotide sequence complementary to the above nucleotide sequence; and

(6′) DNA consisting of a nucleotide sequence which contains mismatch of 1 or more base(s) to the DNA of (6), or a nucleotide sequence complementary to the above nucleotide sequence.

16. The DNA array according to Embodiment 15, wherein the DNAs of (5) has a nucleotide sequence of any one of SEQ ID NOs:75337 to 82784, or SEQ ID NOs:82785 to 166153.

17. The DNA array according to any one of Embodiments 12-16, which comprises, in addition to at least one group of DNAs selected from (1) to (4), DNAs selected from at least one group of (7) to (10):

(7) DNA consisting of a nucleotide sequence which contains mismatch of 1 or more base(s) to the DNA of (1), or a nucleotide sequence complementary to the above nucleotide sequence;

(8) DNA consisting of a nucleotide sequence which contains mismatch of 1 or more base(s) to the DNA of (2), or a nucleotide sequence complementary to the above nucleotide sequence;

(9) DNA consisting of a nucleotide sequence which contains mismatch of 1 or more base(s) to the DNA of (3), or a nucleotide sequence complementary to the above nucleotide sequence; and

(10) DNA consisting of a nucleotide sequence which contains mismatch of 1 or more base(s) to the DNA of (4), or a nucleotide sequence complementary to the above nucleotide sequence.

18. The DNA array according to Embodiment 17, which comprises, in addition to at least one group of DNAs selected from (1) to (4), DNAs selected from at least one group of (7-1) to (10-1):

(7-1) DNA consisting of a nucleotide sequence which contains mismatch of 1 base to the DNA of (1), or a nucleotide sequence complementary to the above nucleotide sequence;

(8-1) DNA consisting of a nucleotide sequence which contains mismatch of 1 base to the DNA of (2), or a nucleotide sequence complementary to the above nucleotide sequence;

(9-1) DNA consisting of a nucleotide sequence which contains mismatch of 1 base to the DNA of (3), or a nucleotide sequence complementary to the above nucleotide sequence; and

(10-1) DNA consisting of a nucleotide sequence which contains mismatch of 1 base to the DNA of (4), or a nucleotide sequence complementary to the above nucleotide sequence.

19. The DNA array according to any one of Embodiments 12-18, which comprises at least one group of DNAs selected from (1) to (4), at least one group of DNAs selected from (7) to (10), DNAs of (5) and/or (6) and DNAs of (5′) and/or (6′).

20. The DNA array according to any one of Embodiments 12-19, wherein the DNAs are oligonucleotides consisting of 10-30 nucleotides.

21. Use of the DNA array according to any one of Embodiments 12-20 for an analyzing method of genes of an industrial yeast.

22. A method for classifying an industrial yeast comprising

(a) hybridizing genomic DNA prepared from the industrial yeast to the DNA array of any one of Embodiments 15-20; and

(b) calculating percentage to hybridize DNAs of any of (1) to (4), and percentage to hybridize DNAs of (5) and/or (6).

23. The method for classification according to Embodiment 22, wherein the DNA array of Embodiment 19 is used, and

percentage of genes which hybridize to DNAs of (1) and/or (2), but not to DNAs of (7) and/or (8) is determined as percentage to hybridize DNAs of any of (1) to (4), and

percentage of genes which hybridize to DNAs of (5) and/or (6), but not to DNAs of (5′) and/or (6′) is determined as percentage to hybridize DNAs of any of (5) and/or (6).

24. A method for detecting nucleotide polymorphism in genome of an industrial yeast comprising

(a) hybridizing genomic DNA prepared from the industrial yeast to the DNA array of any one of Embodiments 17-20; and

(b) selecting a gene wherein hybridization intensity to the DNAs of any of (7) to (10) is higher than hybridization intensity to the DNAs of any of (1) to (4).

25. A method for screening a useful strain of industrial yeast, comprising

(a) hybridizing genomic DNA prepared from the industrial yeast strain to the DNA array of any one of Embodiments 12-20; and

(b) determining the industrial yeast strain as useful when the strain contain 1 or more genes wherein hybridization intensity thereof is 1.5 times higher, or ⅔ or less compared to an average hybridization intensity to the DNAs of any of (1) to (4).

26. A method for screening a gene of an industrial yeast, comprising

(a) hybridizing genomic DNA prepared from the industrial yeast strain to the DNA array of any one of Embodiments 12-20; and

(b) selecting a gene wherein hybridization intensity thereof is 1.5 times higher, or ⅔ or less compared to an average hybridization intensity to the DNAs of any of (1) to (4).

27. A method for screening a gene of an industrial yeast, comprising

(a-1) hybridizing genomic DNA, cDNA or cRNA prepared from the industrial yeast to the DNA array of any one of Embodiments 12-20;

(a-2) independently from the step of (a-1), hybridizing genomic DNA, cDNA or cRNA prepared from the industrial yeast of the strain used for preparing the DNA array to the DNA array of any one of Embodiments 12-20; and

(b) selecting a gene wherein hybridization intensity thereof to the DNAs of any of (1) to (4) in the step (a-1) is significantly different from hybridization intensity thereof in the step (a-2).

28. A method for screening a gene of an industrial yeast, comprising

(a-1) hybridizing cDNA or cRNA prepared from the industrial yeast to the DNA array of any one of Embodiments 12-20;

(a-2) independently from the step of (a-1), hybridizing cDNA or cRNA prepared from another industrial yeast to the DNA array of any one of Embodiments 12-20, wherein the industrial yeast of (a-2) has been cultured in a different condition from the culture condition for the industrial yeast of (a-1); and

(b) selecting a gene wherein hybridization intensity thereof to the DNAs of any of (1) to (4) in the step (a-1) is significantly different from hybridization intensity thereof in the step (a-2).

29. A gene obtained by the screening method of any one of Embodiments 26-28.

30. The gene according to Embodiment 9 or 29 characterized in that concentration of sulfite in a culture medium of an industrial yeast increases when the gene is expressed in the yeast.

31. A nucleic acid encoding a polypeptide of any one of the following i) and ii):

i) a polypeptide having the amino acid sequence represented by SEQ ID NO:3, and

ii) a polypeptide having an amino acid sequence wherein one or more amino acid residue(s) is deleted, substituted and/or added to the amino acid sequence represented by SEQ ID NO:3, and having an activity to increase concentration of sulfite in a culture medium of an industrial yeast when the gene is expressed in the yeast.

32. The nucleic acid according to Embodiment 31, which is selected from the following a) and b):

(a) a nucleic acid having the nucleotide sequence represented by SEQ ID NO:1; and

(b) a nucleic acid having a nucleotide sequence which hybridizes to the nucleotide sequence complementary to the nucleotide sequence represented by SEQ ID NO:1 under a stringent condition, and encodes a polypeptide having an activity to increase concentration of sulfite in a culture medium of an industrial yeast when the gene is expressed in the yeast.

33. A recombinant vector containing the gene of Embodiment 9 or Embodiments 29-30, or the nucleic acid of Embodiment 31-32.

34. A transformant comprising the recombinant vector of Embodiment 33.

35. The transformant according to Embodiment 34, which is a yeast of genus Saccharomyces.

36. A polypeptide of any one of the following i) and ii):

i) a polypeptide having the amino acid sequence represented by SEQ ID NO:3; and

ii) a polypaptide having an amino acid sequence wherein one or more amino acid residue(s) is deleted, substituted and/or added to the amino acid sequence represented by SEQ ID N:3, and having an activity to increase concentration of sulfite in a culture medium of an industrial yeast when the gene is expressed in the yeast.

37. The polypeptide according to Embodiment 36, which is encoded by a nucleic acid selected from the following a) and b):

(a) a nucleic acid having the nucleotide sequence represented by SEQ ID NO:1; and

(b) a nucleic acid having a nucleotide sequence which hybridizes to the nucleotide sequence complementary to the nucleotide sequence represented by SEQ ID No.1 under a stringent condition, and encodes a polypeptide having an activity to increase concentration of sulfite in a culture medium of an industrial yeast when the gene is expressed in the yeast.

38. A method for producing an alcohol or an alcoholic beverage, using the transformant of Embodiment 34 or 35.

39. A breeding method of yeast which is suitable for the production of an alcohol or an alcoholic beverage, characterized in that, expression of the gene of Embodiment 9 or Embodiments 29-30, or the nucleic acid of Embodiments 31-32 is controlled.

40. The breeding method according to Embodiment 39, wherein the yeast belongs to the genus Saccharomyces.

41. Yeast obtained by the breeding method of Embodiment 39 or 40.

42. A method for producing an alcohol or an alcoholic beverage using the yeast mentioned in Embodiment 41.

43. An alcohol or an alcoholic beverage which is produced by the producing method of Embodiment 42.

### BRIEF DESCRIPTION OF THE DRAWING

FIG. 1 shows total chromosome structures of bottom fermenting yeast. A white bar represents an Sc type chromosome while a black bar represents a non-Sc type chromosome. An ellipse represents a centromere. Roman numerals show chromosome numbers for the corresponding S. cerevisiae. In a drawing which shows a non-Sc chromosome, a part marked out in black shows that ligation takes place at the region. For example, in nonScII-nonScIV, it is shown that nonScII and nonScIV are ligated at the part marked out in black (300 kb).

FIG. 2 shows a distribution of identify of the DNA sequence at both ends of 3648 cosmids prepared from the genomic DNA of strain 34/70 with the genome sequence of S. cerevisiae. The X-axis shows the identity to S. cerevisiae and, for example, 84% on the X-axis shows an identity of more than 82% and not more than 84%. The Y-axis shows the numbers of cosmid end sequences showing the identity.

FIG. 3 shows a mapping example of cosmid and shotgun clones to genome sequence of S. cerevisiae. (1) and (2) show genes existing on Watson strand and Crick strand on the chromosome XVI of S. cerevisiae, respectively. (3) and (4) show Sc type and non-Sc type DNA fragments inserted in cosmid clones, respectively. (5) and (6) show Sc-type and non-Sc type DNA fragments inserted in shotgun clones, respectively.

FIG. 4 shows a mapping example of contigs to the genome sequence of S. cerevisiae. (A) is a schematic depiction of Chromosome XVI of S. cerevisiae. (B) is a drawing where the parts of 857 to 886 kb of the Chromosome XVI of S. cerevisiae is enlarged. Y-axis indicates % identity of contigs against S. cerevisiae genome sequence. X-axis indicates position of contigs against S. cerevisiae genome sequence. Contigs (solid lines) are connected with the forward-reverse links (dot lines) from the shotgun and cosmid reads, respectively.

FIG. 5 shows the result of DNA array-based comparative genomic hybridization. The genomic DNA of strain 34/70 was hybridized to a DNA array (Affymetrix Gene Chip Yeast Genome S98 Array) and the signal of each ORF (open reading frame) was normalized to that of the haploid strain S288C and shown as Signal Log Ratio (2ⁿ). Signal Log Ratios were lined following gene order in Chromosome XVI. The non-Sc type genes do not hybridize to this Sc type array, therefore, the points (indicated by arrows) where the Signal Log Ratios show vigorous changes were considered to be translocation sites.

FIG. 6 shows the structure of the Chromosome XVI of strain 34/70 deduced from DNA array and PCR analysis.

FIG. 7 shows the fermentation profiles of SSU1 disruptants and parental strain (BH96). a) shows yeast growth (OD 600), b) shows the change of apparent extract (w/w %) and c) shows sulfite concentration (ppm).

FIG. 8 shows the fermentation profiles of SSU1 overexpressed strains and parental strain (BH225). a) shows yeast growth (OD 600), b) shows the change of apparent extract (w/w %) and c) shows sulfite concentration (ppm).

FIG. 9 shows the change of sulfite concentration during fermentation using MET14 overexpressed strains and parental strains (KN009F and FOY227).

FIG. 10 shows DNA sequences of ScSSU1 and non-ScSSU1.

FIG. 11 shows DNA sequences of ScMET14 and non-ScMET14.

FIG. 12 shows the fermentation profiles of strain 34/70a) shows yeast growth (OD 600) and b) shows the change of apparent extract (w/w %).

### EMBODIMENTS FOR CARRYING OUT THE INVENTION

### I. A Method for Analyzing Gene of an Industrial Yeast

The present invention provides a method for analyzing gene of an industrial yeast. The present method comprises

(a) determining nucleotide sequence of the whole genome sequence of the industrial yeast; and

(c-1) selecting a gene of the industrial yeast encoding an amino acid sequence having 70 to 97% identity to an amino acid sequence encoded by the gene of Saccharomyces cerevisiae, or

(c-2) selecting a gene of the industrial yeast consisting of a nucleotide sequence having 60 to 94% identity to the nucleotide sequence of the gene of Saccharomyces cerevisiae.

In a preferred embodiment, the method of the present invention comprises, (b) comparing the genome sequence with the whole genome sequence of Saccharomyces cerevisiae, after the step (a) and before the step (c-1) or (c-2).

In a preferred embodiment, the analyzing method of the present invention further comprises the step of d) carrying out functional analysis of the selected gene, after the step (c-1) or (c-2).

With regard to the industrial yeast in the present invention, brewing yeast for beer, wine, sake, etc. and yeasts used for the production of fuel alcohols are exemplified. To be more specific, yeast of genus Saccharomyces, etc. may be listed, and in the present invention beer yeasts such as Saccharomyces pastorianus Weihenstephan 34/70, NCYC456, NBRC 1951, NBRC 1952, NBRC 1953, NBRC 1954, etc. may be used. It is also possible to use whisky yeasts such as S. cerevisiae NCYC 90, etc., wine yeasts such as Kyokai wine yeast No. 1, No. 3, No. 4, etc., sake yeasts such as Kyokai sake yeast No. 7, No. 9, etc. and the like.

Preferably, the industrial yeast of the present invention is a brewing yeast, more preferably, a beer yeast. Most preferably, the industrial yeast of the present invention is a bottom fermenting yeast. The bottom fermenting yeast means a yeast used for the lager beer production. Some strains of beer yeasts, such as Saccharomyces pastorianus Weihenstephan 34/70, NCYC456 or the like are classified as bottom fermenting yeasts. The strain used in the following Examples is one of the bottom fermenting yeasts commonly used for the lager beer production. The most of the bottom fermenting yeasts seem to have almost the same genome with that of strain 34/70.

It is also possible to breed yeast having an excellent brewing character when the gene obtained by the method of the present invention is used for carrying out an expression control in such a way that the gene is overexpressed in the yeast, and/or the gene is disrupted. Accordingly, the gene which is obtained by the method of the present invention, peptide which is encoded by the gene, a breeding method of an industrial yeast using the gene, yeast which is obtained by the breeding method, and a method for the production of an alcohol or an alcoholic beverage using the yeast are also within a scope of the present invention.

(A) Determination of Nucleotide Sequence of the Whole Genome Sequence of Industrial Yeast

Determination of the whole genome sequence of an industrial yeast can be achieved by any conventional methods. For example, it includes the steps of (a) genomic DNA is prepared from yeast, (b) shotgun library and (c) cosmid library are prepared from those genomic DNA, (d) DNA fragments to be used for determination of DNA sequence are prepared from those library clones, (e) DNA sequence of the library DNA fragments is determined by a sequence reaction and (f) the sequences of those DNA fragments are assembled to reconstruct the whole genome sequence. Both or only one of (b) shotgun library and (c) cosmid library may be prepared for this purpose.

There is no particular limitation for the methods used for (a) to (f) and the method may be conducted according to the known means, while preferred method for each of them is mentioned below.

(a) Preparation such as extraction, purification, etc. of the genomic DNA is preferably carried out in accordance with the known methods, for example, in “Yeast, a practical approach (IRL Press, 6.2.1, p. 228)” and “Seibutukagakujikkennhou, No. 39, Experiments in Yeast Molecular Genetics (edited by Yasuharu Oshima, Gakkai Shuppan Center, pages 84 to 85, 1996)”. The specific examples of the preferred method for the preparation of DNA are mentioned below.

Yeast cells for the preparation of genomic DNA are cultured by a common method. With regard to a medium, any of natural and synthetic media may be used so far as the medium contains carbon source,-nitrogen source, inorganic salt, etc. which are able to be metabolized by the yeast, whereby cultivation of the microorganism can be efficiently carried out. For example, YPD medium (2% (w/w) glucose, 1% (w/w) yeast extract and 2% (w/w) polypeptone) may be used. With regard to a method of incubation, incubation by shaking at about 25 to 35° C. through the night is recommended.

After the cultivation, cells are recovered from the culture medium by centrifugation. The resulting cell pellet is washed with a washing solution. Example of the washing solution is buffer A (50 mM sodium phosphate, 25 mM EDTA and 1% (v/v) β-mercaptoethanol; pH 7.5), etc. Preparation of the genomic DNA from the washed cells may be carried out according to a common preparation method of genomic DNA where cell walls are lysed using Zymolyase and SDS; protein, etc. are removed using a phenol and phenol/chloroform solution; and genomic DNA is precipitated using ethanol or the like. To be more specific, the following method may be exemplified.

Cultivated cells are washed and resuspended in buffer A, then about 5 to 10 mg of Zymolyase 100T (Seikagaku Kogyo) are added and the mixture is gently shaken at about 25 to 40° C. for about 30 minutes to 2 hours. After the shaking, buffer containing SDS such as buffer B (0.2 M Tris-HCl, 80 mM EDTA and 1% SDS; pH 9.5) is added thereto and the mixture is allowed to stand at about 60 to 70° C. for about 30 minutes to lyse the cells. After that, the cell lysate is cooled on ice, mixed with 5 M potassium acetate and allowed to stand on ice for about 60 minutes further. The resulting solution is centrifuged (for example, at 5,000 g for 10 minutes at 15° C.) to take supernatant. The same volume of ethanol is added to the supernatant to precipitate DNA and the mixture is immediately centrifuged (for example, at 5,000 g for 10 minutes at 15° C.) to obtain DNA. The resulting precipitate is washed with 70% (v/v) ethanol, subjected to natural drying and dissolved in a solution such as TE buffer (10 mM Tris-HCl and 1 mM EDTA; pH 8.0) to give a crude genomic DNA solution. Cesium chloride and bisbenzimide are added to and dissolved in the crude genomic DNA solution, the mixed solution is subjected to an ultracentrifugal separation (for example, at 100,000 g for 17 hours at 25° C.), irradiation with UV light is conducted so that the DNA bands are visualized and the lower band is recovered. Bisbenzimide is removed by extracting the recovered DNA solution with isopropanol which is saturated with cesium chloride solution, then 4-fold by volume of 0.3 M sodium acetate are added to the recovered aqueous layer followed by mixing and the DNA is precipitated by ethanol and recovered by centrifugation. The recovered DNA is treated with RNase and extracted with phenol/chloroform and DNA is purified from the recovered aqueous layer by precipitation with ethanol again. The precipitate recovered by centrifugation is washed with 70% (v/v) ethanol, subjected to natural drying and dissolved in a TE buffer to prepare the genomic DNA solution.

(b) Preparation of a Shotgun Library

As to a method for the preparation of a genomic DNA library using the genomic DNA of yeast prepared in the above (a), a method mentioned in “Molecular Cloning, A, Laboratory Manual, Third Edition-(2001)” (hereinafter, abbreviated as “Molecular Cloning, Third Edition”) may be used and, with regard to a method for the preparation of a shotgun library which is particularly suitable for the determination of the whole genome sequence, the following method may be exemplified.

A TE buffer is added to the genomic DNA prepared in (a) and the genomic DNA is fragmented using Hydroshear (manufactured by GeneMachines) or the like. Terminal of the genome fragment is blunted using a DNA Blunting Kit (manufactured by Takara Shuzo) or the like, and fractionated by means of an agarose gel electrophoresis. Then, genome fragments of about 1.5 to 2.5 kb are excised from the gel and a buffer for the elution of DNA such as an MG-elution buffer (0.5 mol/L ammonium acetate, 10 mmol/L magnesium acetate, 1 mmol/L EDTA and 0.1% SDS) or the like is added to the gel followed by shaking at about 25 to 40° C. through the night to elute DNA. The DNA eluate is treated with phenol/chloroform and precipitated with ethanol to give a genomic library insert. All of the above-mentioned insert and an appropriate vector such as pUC 18 SmaI/BAP (manufactured by Amersham Biosciences) are subjected to ligation using T4 ligase (manufactured by Takara Shuzo) at about 10 to 20° C. for about 20 to 50 hours. The ligation reaction product is precipitated with ethanol and the resulting recombinant vector DNA is dissolved in an appropriate amount of TE buffer. By means of electroporation or the like, the recombinant vector DNA is transformed to Escherichia coli such as an Electro Cell DH5Q strain (manufactured by Takara Shuzo). It is recommended that the electroporation is carried out under the condition mentioned in the attached experimental manual.

The transformants into which recombinant vector containing the genomic DNA fragments is inserted are selected on an appropriate selective medium. For example, when pUC 18 SmaI/BAP is used as a vector, the transformants form white colonies on an LB plate medium (an LB medium (10 g/L of bactotryptone, 5 g/L of yeast extract and 10 g/L of sodium chloride; pH 7.0) which contains 1.6% of agar) containing about 0.01 to 0.1 mg/mL of ampicillin, about 0.1 mg/mL of X-gal and about 1 mmol/L of isopropyl-β-D-thiogalactopyranoside (IPTG) upon incubation through the night at about 30 to 37° C. and, therefore, the selection is easy. The transformants are cultured in LB medium containing about 0.1 mg/mL of ampicillin through the night at about 30 to 37° C. using a 384-well titer plate, a 50% aqueous solution of glycerol in the same volume as the LB is added thereto and the mixture is stirred to give a glycerol stock. Usually, the glycerol stock can be preserved at about −80° C.

(c) Preparation of a Cosmid Library

The genomic DNA prepared in (a) is subjected to a partial digestion using an appropriate restriction enzyme such as Sau3AI (manufactured by Takara Shuzo). It is possible to insert the DNA fragment digested by Sau3AI into a BamHI site of a cosmid vector such as Super CosI vector (manufactured by Stratagene). The treatment with the restriction enzyme and the ligation may be carried out according to the protocol attached thereto. The ligated product obtained by such a method is subjected to a packaging using, for example, Gigapack III Gold (manufactured by Stratagene), and according to the manual for the experimental procedure attached thereto, it is introduced into Escherichia coli such as an XL1-Blue MR strain (manufactured by Stratagene). That is spread on an LB plate medium containing ampicillin and incubated through the night at about 30 to 37° C. to get transformants. The resultant transformants are cultured in LB medium containing about 0.1 mg/mL of ampicillin through the night at about 30 to 37° C. using a 96-well titer plate, a 50% aqueous solution of glycerol in the same volume as the LB is added thereto and the mixture is stirred to give a glycerol stock. Usually, the glycerol stock can be preserved at about −80° C.

(d) Preparation of DNA Fragment for Determination of DNA Sequence

The whole genome sequence of industrial yeast can be determined mainly using the whole genome shotgun method. The DNA fragment of which DNA sequence is determined can be prepared by a PCR using the shotgun library prepared in the above (b). To be specific, clone of the genome shotgun library is inoculated using a replicator (manufactured by Gene Solution) to a 384-well titer plate where about 50 μl each of an ampicillin-containing LB medium is placed to each well and cultured without shaking through the night at about 30 to 37° C. The culture is transferred using a replicator (manufactured by Gene Solution) or the like to a 384-well reaction plate (manufactured by AB Gene) where about 10 μl each of a reaction solution for PCR (TaKaRa Ex Taq manufactured by Takara Shuzo) is placed, and PCR is carried out according to a protocol by Makino, et al. (DNA Research, volume 5, pages 1 to 9 (1998)) or the like using a GeneAmp PCR System 9700 (manufactured by Applied Biosystems) or the like, whereupon amplification of the inserted fragment is carried out.

Excessive primer and nucleotide are removed using a kit for the purification of PCR products (manufactured by Amersham Bioscience), etc. and a sequence reaction is carried out using the sample as a template.

Determination of the genome sequence can also be performed by preparing and using a cosmid library. Cosmid DNA from the cosmid library of (c) can be prepared by the following method. That is, clone derived from cosmid library is inoculated to each well of a 96-well plate where about 1.0 mL each of an ampicillin-containing appropriate medium such as a 2×YT medium (1.6% bactotryptone, 1% yeast extract and 0.5% sodium chloride; pH 7.0) is placed and cultured with shaking through-the night at about 30 to 37° C. Cosmid DNA from the said culture can be prepared using KURABO PI-1100 AUTOMATIC DNA ISOLATION SYSTEM (manufactured by KURABO) according to a manual of KURABO or the like, and they can be used as templates for sequencing reaction.

(e) Sequencing Reaction

A Sequencing reaction can be carried out using a commercially available sequence kit, etc. Preferred examples of the present invention are shown below.

A sequence reaction mixture can be prepared as follows. The PCR product or cosmid DNA prepared in the above (d) is mixed with about 2 μl of DYEnamic ET Terminator Sequencing Kit (manufactured by Amersham Bioscience) and appropriate primers to give about 8 μl of reaction mixture. An M13 forward (M13-21) primer and an M13 reverse (M13RV) primer (manufactured by Takara Bio), etc. are used for the sequence reaction of a PCR product derived from shotgun DNA, while a forward primer such as SS-cos F.1 (SEQ ID NO: 7) and a reverse primer such as SS-cos R.1 (SEQ ID NO: 8), etc. are used for cosmid DNA. Amounts of the primer and the DNA fragment are about 1 to 4 pmole and about 50 to 200 ng, respectively.

A dye terminator sequence reaction of about 50 to 70 cycles can be carried out using the reaction solution and GeneAmp PCR System 9700 (manufactured by Applied Biosciences). When a commercially available kit such as DYEnamic ET Terminator Sequencing Kit is used, a cycle parameter follows a manual attached thereto. Purification of the sample is carried out according to the manual of Millipore using MultiScreen HV plate (manufactured by Millipore), etc. The purified reaction product is precipitated with ethanol and the resulting precipitate is dried and stored in a dark place of about 4° C. The dried product is analyzed using commercially available sequencer and analyzer such as MegaBACE 1000 Sequencing System (manufactured by Amersham Bioscience) and ABI PRISM 3700 DNA Analyzer (manufactured by Applied Biosystems), etc. according to the manuals attached thereto.

(f) Reconstruction of Genome Sequence by Means of Assembly (A Process whereby the Order of Multiple Sequenced DNA Fragments is Determined)

Reconstruction of genomic DNA may be carried out from sequence information of DNA fragments obtained in the above (e). All operations of the reconstruction of genomic DNA sequence can be carried out on an UNIX® platform. Base call can be conducted by a software such as phred (The University of Washington) or the like, masking of vector sequence can be carried out by a software such as Cross Match (The University of Washington) or the like and assembly can be carried out by a software such as Phrap (The University of Washington) or the like. Contig obtained as a result of assembly can be analyzed using a graphical editor such as consed, a graphical editor (The University of Washington) or the like. A series of works from base call to assembly can be carried out en bloc utilizing phredPhrap, a script attached to the consed.

(B) Comparison of the Whole Genome Sequence of the Industrial Yeast with that of S. cerevisiae

Optionally, comparison of the whole genome sequence obtained in (A) with that of S. cerevisiae can be carried our by any known means for comparing two sequences. It may include (g) Preparation of a comparative database compiling the comparison data of each of DNA sequences of both ends of cosmid and shotgun clone and contig with S. cerevisiae genome sequence, and mapping of them on S. cerevisiae genome sequence.

(g) Preparation of a Comparative Database Compiling the Comparison Data of Each of DNA Sequences of Both Ends of Cosmid and Shotgun Clone and a DNA Sequence of Contig with Genomic DNA Sequence of S. cerevisiae, and Mapping of them on S. cerevisiae Genome Sequence.

Widely used industrial yeast such as bottom fermenting yeast (S. pastorianus) has been regarded as a natural hybrid of S. cerevisiae and its closely related species (such as S. bayanus) “Int. J. Syst. Bacteriol. volume 35, pages 508-511 (1985)”. In view of the above, DNA sequences of the both ends of cosmid clone prepared in (e) are subjected to a homology searching against S. cerevisiae genome sequence by a homology searching algorithm, whereupon the homologous region and the identity of each DNA sequence to S. cerevisiae genome sequence can be determined, thus database can be prepared.

The S. cerevisiae genome sequences are accessible from public available data bank, such as, Saccharomyces Genome Database (SGD: http://genome-www.stanford.edu/Saccharomyces/).The percent identity of two amino acid or two nucleic acid sequences can be determined by visual inspection and mathematical calculation, or more preferably, the comparison is done by comparing sequence information using a computer program. An exemplary, preferred computer program is the Genetics Computer Group (GCG; Madison, Wis.) Wisconsin package version 10.0 program, ‘GAP’ (Devereux et al., 1984, Nucl. Acids Res. 12: 387). The preferred default parameters for the ‘GAP’ program includes: (1) The GCG implementation of a unary comparison matrix (containing a value of 1 for identities and 0 for non-identities) for nucleotides, and the weighted amino acid comparison matrix of Gribskov and Burgess, Nucl. Acids Res. 14:6745, 1986, as described by Schwartz and Dayhoff, eds., Atlas of Polypeptide Sequence and Structure, National Biomedical Research Foundation, pp. 353-358, 1979; or other comparable comparison matrices; (2) a penalty of 30 for each gap and an additional penalty of 1 for each symbol in each gap for amino acid sequences, or penalty of 50 for each gap and an additional penalty of 3 for each symbol in each gap for nucleotide sequences; (3) no penalty for end gaps; and (4) no maximum penalty for long gaps. Other programs used by those skilled in the art of sequence comparison can also be used, such as, for example, the BLASTN program version 2.0.9, available for use via the National Library of Medicine website ncbi.nlm.nih.gov/gorf/wblast2.cgi, or the WU-BLAST 2.0 algorithm. Standard default parameter settings for WU-BLAST 2.0 are described at the following Internet site: sapiens.wust1.edu/blast/blast/#Features. In addition, the BLAST algorithm uses the BLOSUM62 amino acid scoring matix, and optional parameters that can be used are as follows: (A) inclusion of a filter to mask segments of the query sequence that have low compositional complexity (as determined by the SEG program of Wootton and Federhen (Computers and Chemistry, 1993); also see Wootton and Federhen, 1996, Analysis of compositionally biased regions in sequence databases, Methods Enzymol. 266: 554-71) or segments consisting of short-periodicity internal repeats (as determined by the XNU program of Claverie and States (Computers and Chemistry, 1993)), and (B) a statistical significance threshold for reporting matches against database'sequences, or E-score (the expected probability of matches being found merely by chance, according to the stochastic model of Karlin and Altschul (1990); if the statistical significance ascribed to a match is greater than this E-score threshold, the match will not be reported.); preferred E-score threshold values are 0.5, or in order of increasing preference, 0.25, 0.1, 0.05, 0.01, 0.001, 0.0001, 1e-5, 1e-10, 1e-15, 1e-20, 1e-25, 1e-30, 1e-40, 1e-50, 1e-75, or 1e-100.

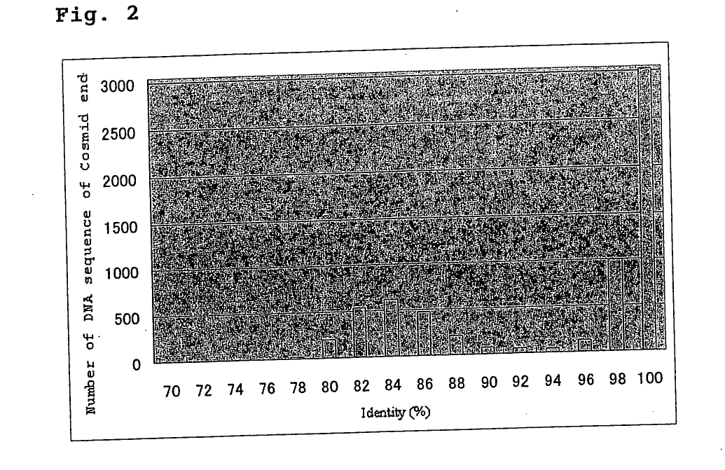

An example of identity percentages distribution graph of cosmid DNA sequence corresponding to S. cerevisiae genomic DNA sequence is shown in FIG. 2. The DNA sequence of cosmid is roughly classified into a DNA sequence group showing more than 94% identity to S. cerevisiae genome sequence and a DNA sequence group showing around 84% identity thereto. Accordingly, a DNA sequence having identity of more than 94% to the nucleotide sequence of the gene of Saccharomyces cerevisiae is named an Sc-type DNA sequence derived from S. cerevisiae. While a DNA sequence having 94% or less identity, more preferably, 60 to 94% identity, most preferably around 84% identity to the nucleotide sequence of the gene of Saccharomyces cerevisiae is named a non-Sc-type DNA sequence derived from a closely-related species of S. cerevisiae. A gene with the Sc type DNA sequence or the non-Sc type DNA sequence is named Sc type gene or non-Sc type gene, respectively.

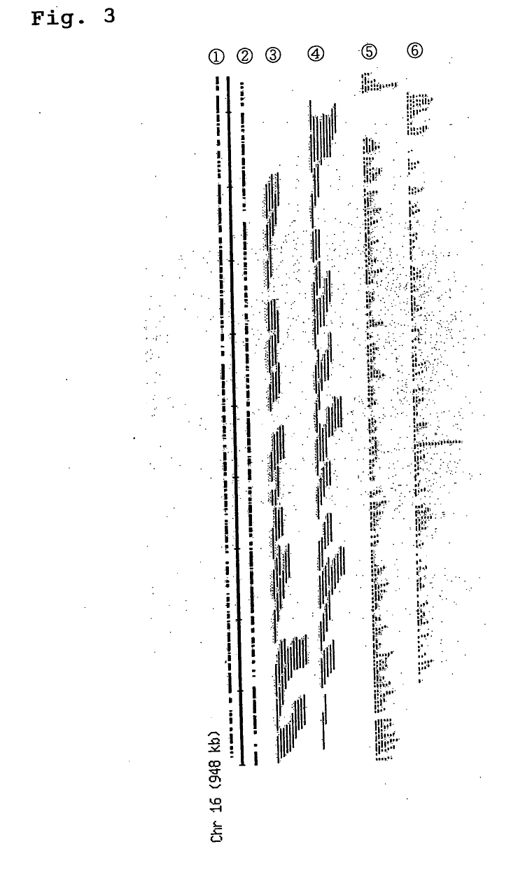

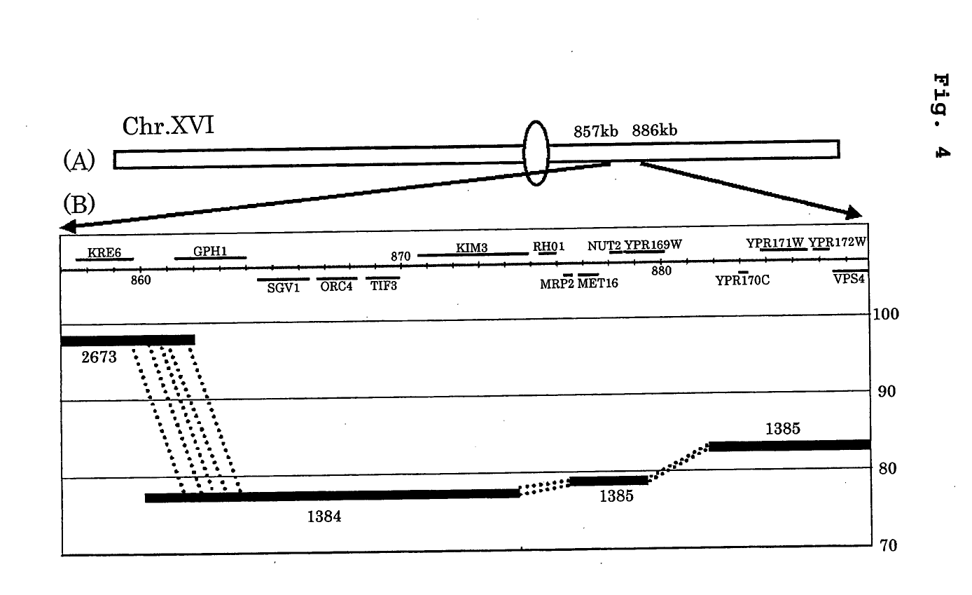

Similarly, a comparative database of the DNA sequence of both ends of shotgun clone prepared in (e) with genomic DNA sequence of S. cerevisiae is prepared. On the basis of the information obtained from the prepared comparative database, a mapping of cosmid clone and shotgun clone on S. cerevisiae genome sequence is carried out (refer, for example, to FIG. 3). A comparative database of the DNA sequence of the contig prepared in (f) with S. cerevisiae genome sequence is also prepared and mapping is carried out. Although the mapping technique is nearly the same as that mentioned above, when contigs linked by paired forward-reverse DNA sequence from the same cosmid and shotgun clone, those contigs are linked (refer, for example, to FIG. 4).

(C)

(C-1) Selection of a gene of the industrial yeast encoding an amino acid sequence having 70 to 97% identity to an amino acid sequence encoded by the gene of Saccharomyces cerevisiae, and

(C-2) selection of-a gene of the industrial yeast consisting of a nucleotide sequence having 60 to 94% identity to the nucleotide sequence of the gene of Saccharomyces cerevisiae.

A stage for the selection of the gene of bottom fermenting yeast encoding an amino acid sequence having 70 to 97% identity to an amino acid sequence encoded by the gene of S. cerevisiae may include (h) a process of identification of ORF (open reading frame) and assignment of function.

(h) Identification of ORF and Assignment of its Function

Identification of ORF in the DNA sequence assembled in (f) is carried out. Preferred examples are specifically mentioned below. With regard to a certain length DNA sequence (such as not less than 150 base) embraced by initiation codon and termination codon, there can be carried out identification of ORF existing in a DNA sequence assembled in (f) using a program, such as ORF finder (http://www.ncbi.nih.gov/gorf/gorf.html) or the like for the identification of ORF for six kinds of reading frames including complementary sequence.

Assignment of function of protein encoded by the identified ORF can be carried out using a homology searching such as BLAST (http://www.ncbi.nlm.nih.gov/BLAST/) or the like to an amino acid sequence of ORF of S. cerevisiae that has been registered and published in the Saccharomyces Genome Database (SGD: http://genome-www.stanford.edu/Saccharomyces/). If necessary, it is possible to analyze the chromosomal structure of an industrial yeast by DNA array-based comparative genomic hybridization and PCR. In the Example 8 in the present specification, the choromosome structure of the S. pastorianus strain 34/70 was analyzed by using a DNA array containing the DNA probes of known S. cerevisiae nucleotide sequences.

Yeast genomic DNA is prepared using a Quiagen Genomic Tip 100/G (#10243) and Qiagen Genomic DNA Buffer Set (#19060) according to the manual attached to the kit. The DNA (e.g., 10 μg) is digested with DNase I (manufactured by Invitrogen) according to a method of Winzeler, et al. (Science, volume 281, pages 1194-1197 (1998)), biotinylated using a terminal transferase (manufactured by Roche) and hybridized to a DNA array (Affymetrix Gene Chip Yeast Genome S98 Array). The DNA array contains the DNA probes of known S. cerevisiae nucleotide sequences. Hybridization and detection of the signal intensity of DNA array are carried out using a Gene Chip Analysis Basic System and analysis soft ware (Microarray Suite 5.0) manufactured by Affymetrix.

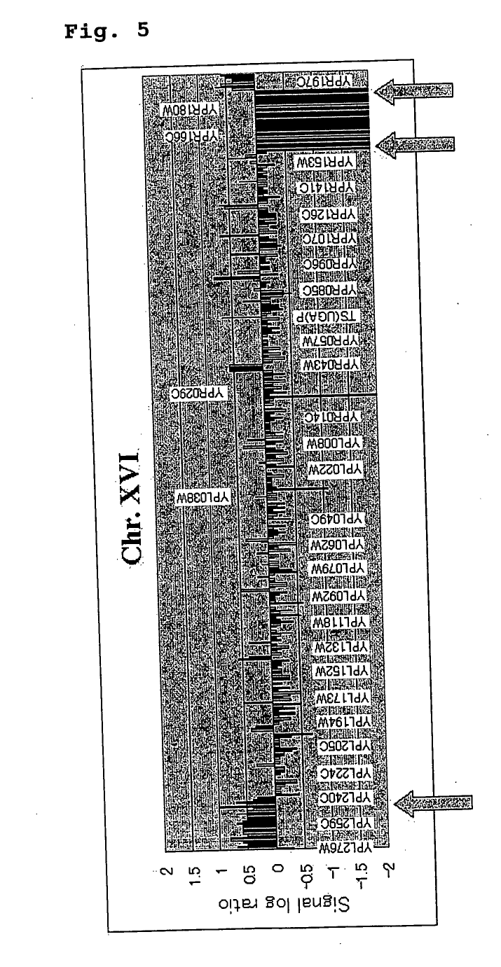

The signal of each probe hybridized with the DNA of brewing yeast is normalized to that of the haploid laboratory yeast strain S288C using an analysis soft ware (Microarray Suite 5.0) and shown as signal log ratio (2ⁿ). Signal log ratios were lined following genes order in each chromosome using a spreadsheet program (Microsoft Excel 2000) and the signal log ratios are shown in bar graphs (refer, for example, to FIG. 5). The non-Sc type genes do not hybridize to the S. cerevisiae array, therefore, the Sc type gene dosage affect the signal log ratio and the points where the signal log ratios show vigorous changes are considered to be translocation sites between Sc type and non-Sc type chromosome.

The chimera chromosome structure can be confirmed by PCR, wherein a genomic DNA derived from brewing yeast is used as a template and Sc type and non-Sc type shotgun sequences are used as primers.

PCR may be carried out using a Takara PCR Thermal Cycler SP according to the attached manual using a Takara LA Taq™ and a buffer attached thereto.

As a result of the PCR, it is confirmed by 0.8% agarose electrophoresis, for example, that a certain length of DNA fragment is amplified from the brewing yeast. When a genomic DNA of S. cerevisiae which is a laboratory strain is used as a template for the PCR, no amplification of DNA fragment is detected. If DNA sequences of the both ends of the DNA fragment amplified from the brewing yeast are further confirmed, it is consistent with the genome sequences determined by a shotgun method and it can be confirmed that, within such region, translocation between Sc type and non-Sc type chromosome takes place, whereupon a chimera chromosome is formed.

Alternatively, a gene of the industrial yeast consisting of a nucleotide sequence having 60 to 94% identity to the nucleotide sequence of the gene of Saccharomyces cerevisiae can be selected. Identity of 60 to 94% in nucleotide sequence level corresponds approximately 70 to 97% in amino acid sequence level. Some nucleotide sequences in non-Sc type chromosome other than open reading frames may have identity of less than 60% to the corresponding nucleotide sequences of Sc type chromosome.

(D) Functional Analysis of the Selected Genes

Further, selected genes may optionally be subjected to functional analysis. For example, the stage of functional analysis of gene may include (i) selection of the gene, (i′) cloning of the gene, (j) functional analysis of the gene by disruption or (k) functional analysis of the gene by overexpression.

(i) Selecting of the Gene

There is no particular limitation for the methods used for the selection of gene(s) for functional analysis. While preferred methods are, for example, a method using the assignment of the function obtained in the above (h) and a method using a DNA array as described below.

One of the purposes of the present invention is to identify genes from industrial yeasts participating in increase in productivity and/or improvement in flavor in the production of an alcohol or an alcoholic beverage. The whole genome sequence of Saccharomyces cerevisiae has been identified, and function of each gene of the of Saccharomyces cerevisiae has already been identified. As mentioned above for the step (h), functions of proteins encoded by the non-Sc type genes can be assigned by a homology searching. As for target genes for functional analysis, non-Sc type genes which are expected from the assignment to have an useful activity, e.g., participating in increase in productivity and/or improvement in flavor in the production of an alcohol or an alcoholic beverage.

For example, the SSU1 gene and the MET14 gene are known to have the sulfite transporting activity, and the adenylsulfate kinase activity, respectively. Both genes are known to play an important role for stabilizing flavor of beer. Other genes from the Sc gene which are known to play some role in a step for producing beer are listed in Table 2 in the following Example 7. These genes, however, do not exert sufficient activity enough for producing tasty beer. Therefore, without any limitation, for the purpose of identifying genes from industrial yeasts participating in increase in productivity and/or improvement in flavor in the production of an alcohol or an alcoholic beverage, non-Sc genes corresponding to these Sc type genes can be selected.

Alternatively, the method using DNA array is, for example, gene expression analysis to identify genes, which show characteristic expression profile under some conditions, or comparative genomic hybridization to identify genes, which have different copy numbers or different DNA sequences, by detecting deference of signal intensities of probes.

(i′) Cloning of the Gene

Genes selected in the above (i) can be obtained from the bottom fermenting yeast according to a common method mentioned, for example, in Molecular Cloning, Third Edition. That is, oligonucleotides having sequences adjacent to the gene are synthesized and a common PCR cloning method is carried out using a genomic DNA prepared from a bottom fermenting yeast as a template, whereupon the selected gene can be isolated and obtained. With regard to DNA sequences obtained as such, for example, SEQ ID NO: 1 may be listed.

When the gene is named, for example, a gene (1), the gene (1) or primer for amplifying the gene (1) by a PCR method may be also synthesized using a polynucleotide synthesizer on the basis of the above-mentioned sequence information.

In addition, gene (1) means not only a DNA fragment having the same DNA sequence as gene (1) but also a DNA fragment hybridizing to the above gene under stringent condition as is defined in the present specification.

The DNA fragment which hybridizes under stringent condition means a DNA fragment which is obtained by a colony hybridization method, a plaque hybridization method, a southern blot hybridization method or the like, using the DNA fragment containing the sequence of the gene (1) identified in the above as a probe.

The term “under stringent condition” means that two nucleic acid fragments can hybridize under a moderately stringent condition or a highly stringent condition. An artisan can easily choose an appropriate moderately or highly stringent condition.

The basic parameters affecting the choice of hybridization conditions and guidance for devising suitable conditions are set forth by Sambrook, Fritsch, and Maniatis (2001, Molecular Cloning: A Laboratory Manual, third edition, Cold Spring Harbor Laboratory Press, Cold Spring Harbor, N.Y., chapters 6 and 7; and Current Protocols in Molecular Biology, 1995, Ausubel et al., eds., John Wiley & Sons, Inc., sections 2.10 and 6.3-6.4), and can be readily determined by those having ordinary skill in the art based on, for example, the length and/or base composition of the DNA.

One way of achieving moderately stringent conditions involves the use of a prewashing solution containing 5×SSC, 0.5% SDS, 1.0 mM EDTA (pH 8.0), hybridization buffer of about 50% formamide, 6×SSC, and a hybridization temperature of about 40-55° C. (or other similar hybridization solutions, such as one containing about 50% formamide, with a hybridization temperature of about 42° C.), and washing conditions of about 60° C., in 0.5×SSC, 0.1% SDS. Preferably, a moderately stringent condition may include hybridization at about 50° C. and 2×SSC.

Highly stringent conditions can also be appropriately selected based on factors like length of DNAs to be hybridized. Generally, such conditions include hybridization and/or washing at higher temperature and/or lower salt concentration (such as hybridization at about 65° C., 6×SCC-0.2×SSC, preferably, 6×SCC, more preferably 2×SSC, most preferably, 0.2×SSC), compared to the moderately stringent condition. For example, highly stringent conditions may include hybridization as defined above, and washing at approximately 68° C., 0.2×SSC, 0.1% SDS. SSPE (1×SSPE is 0.15M NaCl, 10 mM NaH.sub.2 PO.sub.4, and 1.25 mM EDTA, pH 7.4) can be substituted for SSC (1×SSC is 0.15M NaCl and 15 mM sodium citrate) in the hybridization and wash buffers; washes are performed for 15 minutes after hybridization is complete.

It should be understood that the wash temperature and wash salt concentration can be adjusted as necessary to achieve a desired degree of stringency by applying the basic principles that govern hybridization reactions and duplex stability, as known to those skilled in the art and described further below (see, e.g., Sambrook et al., 2001). When hybridizing a nucleic acid to a target nucleic acid of unknown sequence, the hybrid length is assumed to be that of the hybridizing nucleic acid. When nucleic acids of known sequence are hybridized, the hybrid length can be determined by aligning the sequences of the nucleic acids and identifying the region or regions of optimal sequence complementarity.

The hybridization temperature for hybrids anticipated to be less than 50 base pairs in length should be 5 to 25° C., preferably 5 to 10° C. less than the melting temperature (Tm) of the hybrid, where Tm is determined according to the following equations. For hybrids less than 18 base pairs in length, Tm (° C.)=2(number of A+T bases)+4(number of G+C bases). For hybrids above 18 base pairs in length, Tm (° C.)=81.5+16.6(log₁₀ [Na⁺])+0.41(% G+C)−0.63(% formamide−(500/N), where N is the number of bases in the hybrid, and [Na⁺] is the concentration of sodium ions in the hybridization buffer ([Na⁺] for 1×SSC=0.165M) Preferably, each such hybridizing nucleic acid has a length that is at least 15 nucleotides (or more preferably at least 18 nucleotides, or at least 20 nucleotides, or at least 25 nucleotides, or at least 30 nucleotides, or at least 40 nucleotides, or most preferably at least 50 nucleotides), or at least 25% (more preferably at least 50%, or at least 60%, or at least 70%, and most preferably at least 80%) of the length of the nucleic acid of the present invention to which it hybridizes, and has at least 60% sequence identity (more preferably at least 70%, at least 75%, at least 80%, at least 85%, at least 90%, at least 95%, at least 97.5%, or at least 99%, and most preferably at least 99.5%) with the nucleic acid of the present invention to which it hybridizes, where sequence identity is determined by comparing the sequences of the hybridizing nucleic acids when aligned so as to maximize overlap and identity while minimizing sequence gaps as described in more detail above.

The hybridization may be carried out according to a method mentioned in “Molecular Cloning, Third Edition”, “Current Protocols in Molecular Biology, John Wiley & Sons (1987-1997) (hereinafter, abbreviated as Current Protocols in Molecular Biology), “DNA Cloning 1: Core Techniques, A Practical Approach, Second Edition, Oxford University (1995)”, and the like.

To be more specific, shotgun clone containing full-length of the above-mentioned gene (1) can be retrieved using the comparative database obtained in (g) and, on the basis of homology and positional information, etc. When there is no clone containing full-length of the gene in the shotgun library, a DNA fragment encoding the full length of the gene is prepared by a PCR method. For example, a DNA fragment containing the above-mentioned gene is obtained using synthetic DNA primer pair represented by SEQ ID NO: 13 and SEQ ID NO: 14, etc. Similarly, PCR is carried out using a primer pair designed on the basis of the published information of SGD and using genomic DNA of S. cerevisiae or bottom fermenting yeast as a template, whereupon the full length of the Sc type gene corresponding to the non-Sc type gene is prepared. For example, using synthetic oligonucleotides of SEQ ID NO: 15 and NO: 16 as the primer pair, the DNA fragment containing the Sc type gene can be obtained.

Sc or non-Sc type DNA fragment prepared as mentioned above is inserted into, for example, pCR 2.1-TOPO vector attached to a TA cloning kit (Invitrogen) using a TA cloning kit or the like, whereupon a recombinant vector TOPO/Sc gene and TOPO/non-Sc gene containing the DNA fragment having the Sc and the non-Sc type gene, respectively, are able to be prepared. DNA sequences of the Sc and non-Sc type DNA fragments can be comfirmed by a Sanger's method “F. Sanger, Science, volume 214, page 1215, 1981”.

(j) Functional Analysis of the Gene by Disruption

Functional analysis of the selected non-Sc gene may be performed by disruption of the gene, for example. Functional analysis by disruption of the gene can be done by any known conventional methods. In the present specification, “method for disruption of a gene” includes any methods which stop, block, inhibit or suppress at least part of function of the gene, including deletion of the gene, blocking of the gene by antisense nucleic acid, RNAi method or the like. As an example, we will explain disruption of the non-Sc SSU1 gene, which has been identified by the present invention.

According to a method of the document “Goldstein, et al., Yeast, volume 15, page 1541, (1999)”, it is possible to prepare a DNA fragment for gene disruption by PCR where a plasmid containing a drug-resistance gene (such as pFA 6a (G418^{r}), pAG 25 (nat1)) is used as a template. As a primer pair for the PCR, non-ScSSU1_for (SEQ ID NO: 17)/non-ScSSU1_rv (SEQ ID NO: 18) or the like may be used for the non-ScSSU1 disruption. While for the Sc SSU1 disruprion, ScSSU1_for (SEQ ID NO: 19)/ScSSU1_rv (SEQ ID NO: 20) or the like may be used. For the non-Sc type gene disruption, it is also possible to use a plasmid such as pPGAPAUR (AUR1-C) and a primer pair such as non-ScSSU1_for+PGAPAUR (SEQ ID NO: 21)/non-ScSSU1_rv+AURI-C (SEQ ID NO: 22).

An industrial yeast is transformed with the DNA fragment for the gene disruption prepared by the above-mentioned method. The transformation may follow a method mentioned in the Japanese Patent Laid-Open Gazette No. 07/303,475, for example. Further, the concentration of the drug for the selection of transformants may be appropriately determined by investigating the sensitivity of the yeast used as a host.

With regard to the transformant prepared here, it is comfirmed that each of the drug-resistance genes is introduced and the said gene is disrupted correctly using a Southern analysis. To be specific, the genomic DNA extracted from the parental strain and the transformant are firstly digested by an appropriate restriction enzyme to distinguish Sc and non-Sc type gene (for example, at 37° C. for 18 hours), then fractionated with 1.5% agarose gel electrophoresis and transferred to a membrane. After that, they are hybridized to a probe specific to an Sc-type or a non-Sc type gene for example at 55° C. for 18 hours according to a protocol of Alkphos Direct Labelling Reagents (Amersham) and a signal is detected by CDP-Star.

The function of the gene obtained in (i′) can be studied by fermentation test using a parental strain and non-Sc SSU1 disruptants prepared in the above (j) and comparison of their fermentation character. Fermentation test can be carried out, for example, using wort under the following condition.

Original extract: about 10 to 15%

Fermentation scale: 1 to 3 liters

Dissolved oxygen concentration: about 8 to 10 ppm

Fermentation temperature: about 15° C.

Pitching rate: about 4 to 6 g of wet yeast cells/L

Wort is periodically sampled and monitored in cell growth (OD 600), apparent extract, the concentration of the substance participating in the function of the gene obtained in (i′), etc. is analyzed.

For example, when the function of the gene obtained in (i′) participates in discharge of sulfite, the sulfite concentration in the wort during the fermentation is analyzed. Quantitative analysis of sulfite is carried out in such a manner that sulfite is captured in a hydrogen peroxide solution by means of distillation under an acidic condition and subjected to titration with an alkali (Revised Method for BCOJ Beer Analysis by the Brewing Society of Japan).

As candidates for useful genes participating in increase in productivity and/or improvement in flavor in the production of an alcohol or an alcoholic beverage, we may select genes wherein cell growth, apparent extract, and/or the concentration of the substance participating in the function of the gene were substantially changed by disruption of the gene. Especially, change in the concentration of the substance participating in the function of the gene is important and should be monitored.

The “substance participating in the function of the gene” includes, without any limitation, sulfite, ethanol, ester and fusel alcohol or the like. Substances, which are considered to have a negative effect on production of tasty beer, can also be a target of analysis, including diacetyl and phenolic compounds or the like.

(k) Functional Analysis of the Gene by Overexpression

Alternatively, functional analysis of a gene can be achieved by overexpression of the gene.

A DNA fragment containing the full-length of the non-Sc type gene is excised by an appropriate restriction enzyme from the plasmid TOPO/non-Sc gene prepared in (i′). The DNA fragment is inserted into a cloning site of a vector for gene expression such as pNI-NUT to construct a vector (pYI-non-Sc type gene) for overexpression of the non-Sc type gene. The vector pNI-NUT contains URA3 as a homologous recombination site and nourseothricin-resistance gene (nat1) and ampicillin-resistance gene (Amp^{r}) as selective markers. On the other hand, a vector for overexpression of the Sc type gene (pNI-Sc type gene) has a structure where the above-mentioned pYI-non-Sc type gene is substituted by the corresponding Sc type gene. For overexpression of the Sc or non-Sc type gene introduced here, it is preferred to be driven by promoter and terminator of constitutively expressing gene, for example, glyceraldehyde-3-phosphate dehydrogenase gene (TDH3).

An industrial yeast is transformed using the overexpression vector, which is prepared by the above-mentioned method. The transformation is carried out by the method mentioned in the Japanese Patent Laid-Open Gazette No. 07/303,475 and transformants are selected on an appropriate selective medium. Confirmation of the overexpression may be carried out by RT-PCR method, etc. Extraction of the total RNA may be carried out using an RNeasy Mini Kit (Qiagen) or the like, according to the manual of “for total RNA isolation from yeast” attached to the kit. Regarding the SSu1 gene, for example, it is possible to use ScSSU1_for331 (SEQ ID NO: 23)/ScSSU1_{—}982rv (SEQ ID NO: 24) and nonSc-SSU1_for329 (SEQ ID NO: 25)/nonSc-SSU1_{—}981rv (SEQ ID NO: 26) as specific primerpairs for the amplification of Sc and non-ScSSU1 gene, respectively.

To amplify the constitutively expressed gene, for example PDA1, as an internal standard, PDA1_for1 (SEQ ID NO: 27)/PDA1_{—}730rv (SEQ ID NO: 28) etc. may be used as a specific primer pair. PCR product is fractionated with 1.2% agarose gel electrophoresis and detected with ethidium bromide staining. The overexpression of the said gene in the transformant is confirmed by comparison of quantity of the PCR products.

The functional analysis of the gene obtained in (i′) can be carried out by a fermentation test using the parental strain and the overexpressed strain prepared in the above (k). Fermentation test may be carried out under the condition mentioned in (j).

According to the same method mentioned in (j), the wort is periodically sampled and monitored in the cell growth (OD600), apparent extract and the concentration of the substance participating in the function of the gene obtained in (i′).

As candidates for useful genes participating in increase in productivity and/or improvement in flavor in the production of an alcohol or an alcoholic beverage, we may select genes wherein cell growth, apparent extract, and/or the concentration of the substance participating in the function of the gene were substantially changed by overexpression of the gene. Especially, change in the concentration of the substance participating in the function of the gene is important and should be monitored.

(E) Genes and the Gene Library

The present invention also provides genes obtained by the analyzing method of the present invention. The genes of the present invention include both nucleotides of ORF encoding proteins, as well as nucleotides from non-coding regions. Nucleotides from non-coding regions may include regulatory regions, such as promoter, enhancer, silencer, terminator or the like, which are important for regulation of expression of a gene.

Specifically, one embodiment of the present invention is a gene of the industrial yeast encoding an amino acid sequence having 70 to 97% identity to an amino acid sequence encoded by the gene of Saccharomyces cerevisiae, or consisting of a nucleotide sequence having 60 to 94% identity to the nucleotide sequence of the gene of Saccharomyces cerevisiae, which is obtained by the present analyzing method.

The present invention also provides a gene library comprising one or more of genes of the industrial yeast as described above.

Preferably, the gene of the industrial yeast is comprised in, consists of or has any nucleotide sequence selected from SEQ ID NOs:33 to 6236, SEQ ID Nos:166154 to 166181, SEQ ID Nos:166490 to 167042 and SEQ ID Nos:173125 to 174603.

SEQ ID NOs: 33-6236 represent 6204 DNA sequences of non-Sc type ORFs, which can be preferably used as probes of the microarry.

SEQ ID NOs: 166154-166181 represent 28 DNA sequences of mtochondrial ORFs from 34/70 strain, which can be preferably used as probes of the microarry.

SEQ ID NOs: 166490-167042 represent 553 DNA sequences which have not been identified as the above non-Sc ORFs but have significant similarity to the proteins of S. cerevisiae using NCBI-BlastX homology searching.

SEQ ID NOs: 173125-174603 represent 1479 DNA sequences ORFs which have not been identified as the above, but have significant similarity to the proteins of S. cerevisiae using NCBI-BlastP homology searching.

The genes of the present invention also include variants of the natural genes, which may include some modification in one or more nucleotide sequence(s) and/or amino acid sequence(s). The genes of the present invention will be described in detail in the section IV of the present specification.

### II. DNA Array

Embodiments of the Present DNA Array

The present invention also provides a DNA array which is useful for an analyzing method of genes. “Genes” which can be analyzed by using the DNA array of the present invention may include genomic DNA, cDNA or cRNA or the like of an industrial yeast. The present DNA array can also be applied to the ChIP (Chromatin Immuno-Precipitation)-chip method for analyzing binding between DNA and a protein.

The DNA array of the present invention comprises probe(s) of DNA(s) based on the nucleotide sequences of non-Sc genes which have been identified by the present invention. In the present specification, DNA oligonucleotide or cDNA which is fixed on a DNA array, is sometimes called “probe”.

Preferably, the DNA array of the present invention comprises one or more of DNAs, wherein each DNA is selected from at least one group of (1) to (4):

(1) DNA consisting of a nucleotide sequence of an open reading frame of the genome sequence of an industrial yeast which encodes an amino acid sequence having 70 to 97% identity to an amino acid sequence encoded by the gene of Saccharomyces cerevisiae, or a nucleotide sequence complementary to the above nucleotide sequence, or a nucleotide sequence of continuous 10 or more nucleotides selected from the above nucleotide sequences;

(2) DNA consisting of a nucleotide sequence of the genome sequence of an industrial yeast other than from open reading frames which consists of a nucleotide sequence having 60 to 94% identity to the nucleotide sequence of the gene of Saccharomyces cerevisiae, or a nucleotide sequence complementary to the above nucleotide sequence, or a nucleotide sequence of continuous 10 or more nucleotides selected from the above nucleotide sequences;

(3) DNA having a nucleotide sequence of any of SEQ ID Nos.166490 to 167042, or DNA having a nucleotide sequence of any of SEQ ID Nos.167043 to 173124; and

(4) DNA having a nucleotide sequence of any of SEQ ID Nos.173125 to 174603, or DNA having a nucleotide sequence of any of SEQ ID Nos.174604 to 190810. In a preferred embodiment of the present invention, the DNA array comprises DNAs wherein at least one DNA is selected from every group of (1) to (4).

In a preferred embodiments of the present invention, wherein the DNAs of (1) is comprised in, consists of or has any nucleotide sequence selected from SEQ ID NOs:33 to 6236 and SEQ ID NOs:166154 to 166181, and SEQ ID NOs:6237 to 75336 and SEQ ID NOs:166182 to 166489.

SEQ ID NOs: 33 to 6236 correspond to ORF encoding non-SC proteins of the present invention. These sequences can be fixed to the DNA array as cDNA probes. SEQ ID NOs:6237 to 75336 are short nucleotide sequences of about 25 continuous nucleotides selected from each of SEQ ID NOs:1 to 6236 SEQ ID Nos: 166182-166489 represent sequences of oligonucleotides based on DNA sequences of SEQ ID NOs: 166154 to 166181.

The ORF sequences from yeast in the present specification may include intron. Such intron sequences can be easily recognized for an artisan. The term “comprised in”, therefore, means that the nucleotide sequences of the DNA probes on the DNA array can include only exon sequences (without intron) which can be easily selected by referring to the ORF sequences disclosed in the present specification.

The term “have (has)” or “having” means that the nucleotide sequence of the present invention may include additional sequence which does not substantially affect the function of the DNA of the present invention. For example, DNAs encoding ORF may contain additional signal or tag sequence which can facilitate secretion and/or purification of the protein encoded by the ORF. Alternatively, oligonucleotides may contain additional one or several nucleotide sequences at its terminal which does not affect hybridization specificity of the oligonucleotides.

SEQ ID Nos. 167043-173124 represent sequences of oligonucleotides based on DNA sequences of SEQ ID NOs: 166490-167042 SEQ ID NOs: 174604-190810 represent sequences of oligonucleotides based on DNA sequences of SEQ ID NOs: 173125-174603. These sequence can be fixed to the DNA array as oligonucleotide probes.

Another embodiment of the DNA array of the present invention is that of further comprising, in addition to at least one group of DNAs selected from (1) to (4), DNAs of the following (5) and/or (6):

(5) DNA consisting of a nucleotide sequence of an open reading frame of the genome sequence of an industrial yeast which encodes an amino acid sequence having identity of more than 97% to an amino acid sequence encoded by the gene of Saccharomyces cerevisiae, or a nucleotide sequence complementary to the above nucleotide sequence, or a nucleotide sequence of continuous 10 or more nucleotides selected from the above nucleotide sequences; and

(6) DNA consisting of a nucleotide sequence of the genome sequence of an industrial yeast other than from open reading frames which consists of a nucleotide sequence having identity of more than 94% to the nucleotide sequence of the gene of Saccharomyces cerevisiae, or a nucleotide sequence complementary to the above nucleotide sequence, or a nucleotide sequence of continuous 10 or more nucleotides selected from the above nucleotide sequences.

DNAs of (5) correspond to ORF of Sc genes, and DNAs of (6) correspond to non-coding regions of Sc genes, respectively. The DNA array comprising to DNAs of (5) and/or (6) in addition to DNAs of any of (1) to (4), is useful for a method of classifying an industrial yeast, for example. In a preferred embodiment, the DNAs of (5) has a nucleotide sequence of any one of SEQ ID NOs: 75337 to 82784, or SEQ ID NOs: 82785 to 166153. SEQ ID NOs: 75337 to 82784 correspond to ORF encoding non-SC proteins of the present invention. SEQ ID NOs: 82785 to 166153 are short nucleotide sequences of about 25 continuous nucleotides selected from each of SEQ ID NOs: 33 to 6236.

In a preferred embodiment, the DNA array comprises DNAs selected from the group of (1) and/or (2), together DNAs selected from the group of (5) and/or (6). Hybridization intensity of a sample to a DNA probe of (1) is compared to the corresponding DNA probe of (5). Similarly, hybridization intensity of a sample to a DNA probe of (2) is compared to the corresponding DNA probe of (6).

Optionally, the present DNA array of this embodiment may further comprise DNAs of the following (5′) and/or (6′):

(5′) DNA consisting of a nucleotide sequence which contains mismatch of 1 or more base(s) to the DNA of (5), or a nucleotide sequence complementary to the above nucleotide sequence; and

(6′) DNA consisting of a nucleotide sequence which contains mismatch of 1 or more base(s) to the DNA of (6), or a nucleotide sequence complementary to the above nucleotide sequence.

As described above, DNAs of (5) correspond to ORF of Sc genes, and DNAs of (6) correspond to non-coding regions of Sc genes, respectively. DNAs of (5′) and (6′) contain mismatch of one or more base(s), preferably one base to (5) and (6), respectively. In general, a sample of DNAs shows a different hybridization intensity to a probe having mismatch of one or more base(s) in the DNA array hybridization assay. In a preferred embodiment, if a sample DNA hybridizes to a DNA probe of (5) (or (6)) and does not hybridize to the corresponding DNA probe of (5′) (or (6′)), it can be concluded that the sample DNA is positive as regards the DNA probe of (5) of Sc type gene. On the contrary, if a sample DNA hybridizes to a DNA probe of (5) (or (6)) but also hybridizes to the corresponding DNA probe of (5′) (or (6′)), it can be concluded that the sample DNA is negative or pseudopositive as regards the DNA probe of (5) of Sc type gene.

Accordingly, DNAs of (5′) and/or (6′) may be optionally used as DNA probes of the present DNA array for determining and confirming hybridization specificity of a sample to the DNAs of (5) and/or (6).

Still another aspect of the DNA array of the present invention is that of further comprising, in addition to at least one group of DNAs selected from (1) to (4), DNAs selected from at least one group of (7) to (10):

(7) DNA consisting of a nucleotide sequence which contains mismatch of 1 or more base(s) to the DNA of (1), or a nucleotide sequence complementary to the above nucleotide sequence;

(8) DNA consisting of a nucleotide sequence which contains mismatch of 1 or more base(s) to the DNA of (2), or a nucleotide sequence complementary to the above nucleotide sequence;

(9) DNA consisting of a nucleotide sequence which contains mismatch of 1 or more base(s) to the DNA of (3), or a nucleotide sequence complementary to the above nucleotide sequence; and

(10) DNA consisting of a nucleotide sequence which contains mismatch of 1 or more base(s) to the DNA of (4), or a nucleotide sequence complementary to the above nucleotide sequence.

In general, a sample of DNAs shows a different hybridization intensity to a probe having mismatch of one or more base(s) in the DNA array hybridization assay. This type of DNA array, therefore, is useful for a detecting nucleotide polymorphism in genome of an industrial yeast. It can also be useful for the method of screening a useful strain of industrial yeast or useful genes themselves.

In a preferred embodiment, the DNAs of (7) to (10) includes mismatch of only one base. That is, the DNAs of (7) and (10) are the following DNAs of (7-1) to (10-1), respectively.

(7-1) DNA consisting of a nucleotide sequence which contains mismatch of 1 base to the DNA of (1), or a nucleotide sequence complementary to the above nucleotide sequence;

(8-1) DNA consisting of a nucleotide sequence which contains mismatch of 1 base to the DNA of (2), or a nucleotide sequence complementary to the above nucleotide sequence;

(9-1) DNA consisting of a nucleotide sequence which contains mismatch of 1 base to the DNA of (3), or a nucleotide sequence complementary to the above nucleotide sequence; and

(10-1) DNA consisting of a nucleotide sequence which contains mismatch of 1 base to the DNA of (4), or a nucleotide sequence complementary to the above nucleotide sequence.

Preferably, mismatch of one base is at the center or near the center of each DNA.

One of the most preferred embodiments of the present DNA array comprises at least one group of DNAs selected from (1) to (4) (preferably (1)/and/or (2)), at least one group of DNAs selected from (7) to (10) (preferably (7) and/or (8)), DNAs of (5) and/or (6) and DNAs of (5′) and/or (6′). The DNA array of this embodiment if especially useful for the present method for classifying an industrial yeast, as well as a method for analyzing the chromosomal structure of an industrial yeast by DNA array-based comparative genomic hybridization as discussed in the present specification below. Preferably, at least one group of DNAs selected from (7) to (10), and DNAs of (5′) and/or (6′) has a mismatch of only one base.

Probes which are attached to the DNA arrays of the present invention can be cDNA probes or oligonucleotids. There is no limitation to the length of cDNA probes, but preferably, no longer than 1,000 bases. Oligonucleotide probes range form about 5 to about 50 nucleotides, preferably, 10-30 nucleotides.

Production of a DNA Array of this Invention

A DNA array of the present invention can be produced based on the DNA sequence information of the ORFs and/or non-coding regions (intergenic DNA sequence between two ORFs) obtained in the above (f). Examples include a DNA array comprising a solid-support to which at least one of a polynucleotide comprising the DNA sequence obtained above items (f).

DNA arrays of the present invention include substrates known in the art, such as a DNA microarray, a DNA chip, polynucleotide array and a DNA macroarray, or the like, and comprises a solid support and plural polynucleotides of fragments thereof which are adhered to the surface of the solid support. As the polynucleotides or oligonucleotides adhered to the solid support, the polynucleotides or oligonucleotides of the present invention obtained in the above items (f) and (h) can be used.

The analysis described below can be efficiently performed by adhering the polynucleotides or oligonucleotides to the solid support at a high density, though a high fixation density is not always necessary. Apparatus for achieving a high density, such as an arrayer robot or the like, is commercially available from Takara Shuzo (GMS417 Arrayer), and the commercially available product can be used. Also, the oligonucleotide of the present invention can be synthesized directly on the solid support by the photolithography method or the like (Nat. Genet. 21, 20-24 (1999)). In this method, a linker having a protective group which can be removed by light irradiation is first adhered to a solid support, such as slide glass or the like. Then, it is irradiated with light through a mask (a photolithograph mask) permeating light exclusively at a definite part of the adhesion part. Next, an oligonucleotide having a protective group which can be removed by light irradiation is added to the part. Thus, a ligation reaction with the nucleotide arises exclusively at the irradiated part.

By repeating this procedure, oligonucleotides, each having a desired sequence, different from each other can be synthesized in respective parts. Usually, the oligonucleotides to be synthesized have a length of 10 to 30 nucleotides.

Alternatively, cDNA probes which can be prepared by any known conventional methods based on the nucleotide sequences of mRNA obtained from the industrial yeast can be used. A cDNA probe is fixed to the DNA array after it has been prepared. Examples of general methods for preparing and fixing the cDNA probes to the DNA array are explained in “Science volume 270, pages 467-470 (1995)”, for example.

There is no particular limitation for the methods used for the production of DNA array and the method may be conducted according to the known means, while preferred method for each of them is mentioned below.

(1) Production of a DNA Array

(1)-1 Solid Support

Any materials which the polynucleotids or fragments can be adhere to the surface of can be used as the solid supports using the invention DNA array. There is no particular limitation for the material and shape used for the solid support, while preferred materials are some resinoids, such as polycarbonate, plastics or the like, as a material and a plate-like and film-like as a solid.

(1)-2 Selection of an Oligonucleotide

The example of oligonucleotides to be fixed on the plate of a DNA array of this invention are as follows. Based on the DNA sequences of ORFs obtained in the above (h) and/or intergenic DNA sequences deduced from the above (h), unique and perfect complementary probes (PM Probe; Perfect Match Probe) against whole genome sequence of brewing yeast can be designed using a certain method of probe production, such as GeneChip® (Affymetrix) technology or the like.

The oligonucleotide probes range form about 5 to about 50 nucleotides, preferably from about 5 to about 45 nucleotides, still more preferably from about 10 to about 40 nucleotides and most preferably from about 10 to 30 nucletides in length. Particularly preferred arrays contain probes ranging from about 20 to about 25 oligonucleotides in length. The array may comprise more than 10, preferably more than 50, more preferably more than 100, and most preferably more than 1000 oligonucleotide probes specific for each target gene (Where the array refers to the DNA array to be fixed the oligonucleitide probes using a certain method of probe production, such as GeneChip® (Affymetrix) technology or the like). In a preferred embodiment, the array comprises at least 10 different oligonucleotide probes for each gene. In another preferred embodiment, the array 20 or fewer oligonucleotides complementary each gene.

Widely used industrial yeast such as bottom fermenting yeast (S. pastorianus) has been regarded as a natural hybrid of S. cerevisiae and its closely related species (such as S. bayanus) as described above. So the bottom fermenting yeast has at least two different genes whose DNA sequences are similar to each other. Thereby, it is desirable to remove probes that hybridize to transcription products of more than one gene. One can then remove any probes that are similar to any other genes, and make the probe set a probe set for that genes using a certain method of probe production, such as GeneChip® (Affymetrix) technology or the like)

The array may further comprise mismatch control probes. Where such mismatch controls are present, the quantifying step may comprise calculating the difference in hybridization signal intensity between each of the oligonucleotide probes and its corresponding mismatch control probe. The quantifying may further comprise calculating the average difference in hybridization signal intensity between each of the oligonucleotide probes and its corresponding mismatch control probe for each gene. The term mismatch probe refer to probes whose sequence is deliberately selected not to be perfectly complementary complementary to a particular target sequences. For each mismatch (MM) control in a high-density array there typically exists a corresponding perfect match (PM) probe that is perfectly complementary to the same particular target sequences. The mismatch may comprise one or more bases, preferably one base.

While the mismatch(es) may be located anywhere in the mismatch probe, terminal mismatches are less desirable as a terminal mismatch is less likely to prevent hybridization of the target sequences. In a particularly preferred embodiment, the mismatch is located at or near the center of the probe so that the mismatch is most likely to destabilize the duplex with the target sequence under the optimal hybridization conditions. Preferably, the mismatch is adenine (A) to thymine (T) and vice versa, or guanine (G) to cytosine (c) and vice versa.

Nucleotides number of these oligonucleotides are not limited, but 10 to 30 nucleotides are preferable. 8-51 probes an each locus can be designed with focus on 3′ prime side of each locus as the use of sets of probes for each locus provided redundancy in the detection and analysis of the data, can mitigate the potentially confounding effects of occasional cross-hybridization, and can make it so all probes do not have to hybridize identity in order to obtain quantitative information. To further increase the sensitivity and specificity of detection, each PM probe can be designed with a closely related mismatch probe (MM probe) that is identical to PM probe with the exception of a mismatched base, i.e. base 13. The preferable length of oligonucleotide which is used in this invention is 25 base, but no particular limitation. for the length of oligonucleotide. Preferably, the mismatch is adenine (A) to thymine (T) and vice versa, or guanine (G) to cytosine (c) and vice versa.

(1)-3 Adhering Oligonucleotides to Solid Support

There is no particular limitation for the methods used for adhering oligonucleotides to solid support, and the method may be conducted according to the known means, while preferred method is mentioned below. For example, all of designed PM and MM probes as above ((1)-2) can be adhered to the surface of solid support to produce a DNA array using a certain method, such as GeneChip® technology or the like.

There is no particular limitation for the methods used for analysis using DNA maicroarray, while preferred methods for each of them is mentioned below, i.e., the example of gene expression analysis to identify genes, which show characteristic expression profile under some conditions, classification of industrial yeast, detection of nucleotide polymorphism and selection of genes for functional analysis are mentioned below.

### III. Analyzing Methods using the DNA Array

The arrays of the invention can be used for various methods for analyzing genomic DNA, cDNA or cRNA of an industrial yeast. The analyzing methods include, for example, 1) a method for determining expression of each gene of non-Sc type, 2) a method for classifying an industrial yeast, 3) a method for detecting nucleotide polymorphism in genome of an industrial yeast, 4) a method for screening a useful strain of industrial yeasts, and 5) a method for screening a gene of an industrial yeast.

(m) Gene Expression Analysis

Gene expression analysis of an industrial yeast can be carried out using the DNA array of the present invention produced according to the method described in (1). It is possible to identify the highly inducible or reducible gene(s) according to change of not only medium but also environment using the DNA array. It is also possible to identify the specific gene(s) for lager brewing yeast in brewing using the DNA array. But it is not limited for these examples.

Gene expression analysis includes culturing of a industrial yeast, preparation of mRNA, synthesis of labeled cRNA(or cDNA), hybridization, and data analysis. There is no particular limitation for the methods of Gene expression, while preferred methods for each of them is mentioned below.

(m)-1 Culturing a Industrial Yeast in a Various Condition

Industrial yeast can be cultivated under various conditions for any purpose. For example, the cultivation for identification of genes which respond to the change of composition of culture medium can be carried out as mentioned below. Industrial yeast can be grown overnight in a Zinc replete medium, such as LZMM medium+40 μM zinc sulfate at 30° C. with shaking. LZMM medium contains 0.17% yeast nitrogen base w/o amino acids (manufactured by DIFCO), 0.5% ammonium sulfate, 20 mM sodium citrate (pH 4.2), 125 μM MnCl2, 10 μM FeCl2, 2% maltose, 10 mM EDTA (pH 8.0), or the like. Cells are harvested and washed three times with sterile distilled water An adequate amount of cells, are inoculated to an optical density (OD600) of 0.25, or the like, in 1) zinc depleted medium (LZMM medium) or the like, 2) zinc replete medium (LZMM+40 μM zinc sulfate) or the like, 3) oxidative stress medium (LZMM+40 μM zinc sulfate+2 mM H₂O₂) or the like, 4) carbon starvation medium (deleting maltose from above LZMM+40 μM zinc sulfate) or the like. Cells are grown at 30° C. for 6 hours and harvested for RNA preparation. Cells withdrawn from fermentation tube under beer fermenting condition can be used for the following experiments.

(m)-2 Preparation of mRNA

Preparations of total RNA from each cell can be carried out using an RNeasy® Mini Kit (manufactured by QIAGEN) or the like according to a manual. Preparations of Poly (A)+mRNA from each total RNA are carried out using an Oligotex Direct mRNA kit (manufactured by QIAGEN) or the like according to a manual. There is no particular limitation for the methods used for preparation of mRNA and the method may be conducted according to the known means.

(m)-3 Synthesis of Labeled cRNA

Synthesis of Labeled cRNA can be carried out using a BioArray HighYield RNA Transcript Labeling Kit (manufactured by Affymetrix) or the like according to a manual. Biotin can be used for labeling. There is no particular limitation for the methods used for syntheses of Labeled cRNA and the method may be conducted according to the known means.

(m)-4 Hybridization

5 μg of Biotin-Labeled cRNA, 1.7 μl of 3 nM Control Oligonucleotide B2 (manufactured by Affymetrix), 5 μl of 20× Eukaryotic Hybridization Controls (manufactured by Affymetrix), 1 μl of 10 mg/ml Herring Sperm DNA (manufactured by Affymetrix), 1 μl of 50 mg/ml Acetylated BSA (manufactured by Affymetrix), 50 μl of 2× Hybridization buffer (manufactured by Affymetrix), and water (manufactured by Affymetrix) to give final volume of 100 μl are mixed and hybridized to the DNA array according to a Technical Manual of Affymetrix. After 16 hours of hybridization, hybridization cocktail are removed and the DNA array is washed using the a GeneChip® Fludics Station (manufactured by Affymetrix) or the like, and stained with a Streptavidin Phycoerythrin (300 μl of 2× MES Stain Buffer (manufactured by Affymetrix), 24 μl of 50 mg/ml acetylated BSA (manufactured by Affymetrix), 6 μl of 1 mg/ml StreptAvidin-Phycoerythrin (manufactured by Affymetrix), 270 μl of DI Water (manufactured by Affymetrix)) according to a Technical Manual of Affymetrix. There is no particular limitation for the methods used for hybridization and the method may be conducted according to the known means.

(m)-5 Data Analysis

Data analysis of the DNA array can be carried out using a commercially available software (for example, a GCOS (GeneChip Operating Software manufactured by Affymetrix; GeneSpring manufactured by Silicon Genetics; ImaGene manufactured by Takara Shuzo; Array Gauge manufactured by Fuji Photo Film; ImageQuant manufactured by Amersham Pharmacia Biotech, or the like) according to a Technical Manual. Genes which show characteristic expression profile can be identified and selected for functional analysis.

Furthermore, the identified gene can be used as a gene marker to figure out condition of the yeast cells. A fluctuation in the expression amount of a specific gene can be monitored using a nucleic acid molecule obtained in the time course of culture as the nucleic acid molecule derived from brewing yeast. The culture conditions can be optimized by analyzing the fluctuation. The expression profile of the brewing yeast at the total gene level (namely, which genes among a great number of genes encoded by the genome have been expressed and the expression ratio thereof) can be determined using a nucleic acid molecule having the sequences of many genes determined from the whole genome sequence of the yeast. Thus, the expression amount of the genes determined by the whole genome sequence can be analyzed and, in its turn, the biological conditions of the yeast can be recognized as the expression pattern at the full gene level.

As a result of gene expression analysis, a group of genes which shows distinctive expression under a certain condition can be identified as target genes for further functional analysis. “A certain condition” may include, without any limitation, changes in environment, such as, temperature, pH, or osmotic pressure, as well as changes in components of medium. As a screening method for identifying genes showing distinctive expression under the certain condition, for example, a gene wherein significant changes in expression of the gene are recognized according as changes in environment, can be selected. “Significant changes” mean preferably, 1.5 times higher, or ⅔ or less, more preferably, 2 times higher, or ½ or less, even more preferably 3 times higher, or ⅓ or less, but not limited thereto.

There is no particular limitation for the methods used for analysis of data and the method may be conducted according to the known means.

Comparative Hybridization Analysis

The DNA array of the present invention can also be used for comparative hybridization analysis using genomic DNA, cDNA or cRNA as a sample/for hybridization.

(n) Classification of Industrial Yeast

An embodiments of the present invention includes a method for classifying an industrial yeast comprising

(a) hybridizing genomic DNA prepared from the industrial yeast to the DNA array of the present invention; and

(b) calculating percentage to hybridize DNAs of any of (1) to (4), and percentage to hybridize DNAs of (5) and/or (6).

For the purpose of this embodiment, the DNA array comprises, in addition to the DNAs of any of (1) to (4), DNAs of the (5) and/or (6) is preferably used (Embodiment 15 or 16).

Preferably, hybridization intensity to the DNAs of (1) to the DNAs of (5) is compared. Alternatively, hybridization intensity to the DNAs of (2) to the DNAs of (6) is compared.

The present DNA array may optionally comprise DNAs of the (5′) and/or (6′) and/or (7) and/or (8)having one or more mismatch(es) to the DNA of (5) and/or (6) and/or.1 and/or (2), respectively. DNAs of (5′) and/or (6′) and/or . . . and/or . . . may be optionally used as DNA probes of the present DNA array for determining and confirming hybridization specificity of a sample to the DNAs of (5) and/or (6) and/or.1.and/or (2), respectively.

Specifically, in one of preferred embodiment of the present invention, the DNA array comprises at least one group of DNAs selected from (1) to (4) (preferably (1) and/or (2)), at least one group of DNAs selected from (7) to (10) (preferably (7) and/or (8)), DNAs of (5) and/or (6) and DNAs of (5′) and/or (6′). In the method using the above DNA array, percentage of genes which hybridize to DNAs of (1) and/or (2), but not to DNAs of (7) and/or (8) is determined as percentage to hybridize DNAs of any of (1) to (4), and percentage of genes which hybridize to DNAs of (5) and/or (6), but not to DNAs of (5′) and/or (6′) is determined as percentage to hybridize DNAs of any of (5) and/or (6).

It is possible to classify industrial yeast using a DNA array mentioned above. Preparation of yeast genomic DNA and hybridization to a DNA array may be carried out as described before. Detection of the signal intensity of array is carried out using a Gene Chip Analysis Basic System and analysis soft ware (GCOS; GeneChip Operating Software 1.0) manufactured by Affymetrix. The percentage of probes, to which the DNA of an industrial yeast hybridizes, is calculated. The identity between the strain from which the DNA probes of the DNA array are derived (e.g. 34/70) and the tested strain is estimated from the calculation result.

Industrial yeast strains can be classified on the basis of the identity. A strain of industrial yeasts which shows hybridization percentage to the non-Sc genes and the Sc-gene on the DNA array similar to that of another strain can be classified as a close strain of the other strain.

(o) Detection of Nucleotide Polymorphism

An embodiments of the present invention includes a method for detecting nucleotide polymorphism in genome of an industrial yeast comprising

(a) hybridizing genomic DNA prepared from the industrial yeast to the DNA array of the present invention; and

(b) selecting a gene wherein hybridization intensity to the DNAs of any of (7) to (10) is higher than hybridization intensity to the DNAs of any of (1) to (4).

For the purpose of this embodiment, the DNA array comprises, in addition to the DNAs of any of (1) to (4), DNAs of any of (7) to (10) is preferably used (Embodiments 17-19).

It is possible to detect nucleotide polymorphism of a industrial yeast by comparative genomic hybridization with the DNA array mentioned above. For this embodiment, the probes on the array are preferably oligonuclotide probes.

As an illustrative example, the sets of oligonucleotides for each probe consist of Perfect Match oligonucleotide (PM) which is identical to the sequence of strain 34/70 and MisMatch oligonucleotide (MM) which contains a single base mismatch, e.g., in the central position of the oligonucleotide. It is possible to detect nucleotide polymorphism from the gene whose signal intensity in MM is higher (for example, more than 5-fold) than that in PM.

Genes comprising one or more mismatche(s) can be preferable candidates of target genes for further functional analysis.

(p) Selection of Useful Yeast Strains and Genes

An embodiments of the present invention includes a method for screening a useful strain of industrial yeast, comprising

(a) hybridizing genomic DNA prepared from the industrial yeast strain to the DNA array of the present invention; and

(b) determining the industrial yeast strain as useful when the strain contain 1 or more genes, preferably, 2 or more genes, wherein hybridization intensity thereof is 1.5 times higher or less than ⅔ compared to an average hybridization intensity to the DNA of any of (1) to (4).

A gene of genomic DNA which shows hybridization intensity which shows significantly different from other genes may be selected as candidate of useful genes for further functional analysis. Therefore, strains of industrial yeasts comprising 1 or more genes, preferably 2 or more genes can be selected as candidate of useful strains. Preferably, hybridization intensity of such gene is significantly different from that of other genes, more preferably, thereof is 1.5 times higher or less than ⅔ compared to an average hybridization intensity to the DNA of (1) and/or (2). More preferably, the difference in hibridization intensity is 2 times higher, or ½ or less, even more preferably 3 times higher, or ⅓ or less, but not limited thereto.

Further embodiment of the present invention is a method for screening a gene of an industrial yeast, comprising

(a) hybridizing genomic DNA prepared from the industrial yeast strain to the DNA array of the present invention; and

(b) selecting a gene wherein hybridization intensity thereof is 1.5 times higher or less than ⅔ compared to an average hybridization intensity to the DNA of any of (1) to (4).

Further another embodiment of the present invention is a method for screening a gene of an industrial yeast, comprising

(a-1) hybridizing genomic DNA, cDNA or cRNA prepared from the industrial yeast to the DNA array of the present invention;

(a-2) independently from the step of (a-1), hybridizing genomic DNA, cDNA or cRNA prepared from the industrial yeast of the strain used for preparing the DNA array to the DNA array of the present invention; and

(b) selecting a gene wherein hybridization intensity thereof to the DNA any of (1) to (4) in the step (a-1) is significantly different from hybridization intensity thereof in the step (a-2).

The above screening method enable to select genes which hybridization intensity to the DNA array are different between strains. From the results of comparative genomic hybridization analysis, a gene which has probe sets showing low signal intensities may be lost or have different sequence from that of the strain of the probes of the array (e.g., 34/70). In contrast, a gene which has probe sets showing high signal intensities may be high in copy number. For example, as for a gene, which show hybridization intensity of 2 times higher in the step (a-1) than that of the strain used for preparing the DNA array in the step (a-2), the strain of (a-1) is likely to have two or copies of the gene.

Another embodiment of the present invention is a method for screening a gene of an industrial yeast, comprising

(a-1) hybridizing cDNA or cRNA prepared from the industrial yeast to the DNA array of the present invention;

(a-2) independently from the step of (a-1), hybridizing cDNA or cRNA prepared from another industrial yeast to the DNA array of any one of Embodiments 10-19, wherein the industrial yeast of (a-2) has been cultured in a different condition from the culture condition for the industrial yeast of (a-1); and

(b) selecting a gene wherein hybridization intensity thereof to the DNA of any of (1) to (4) in-the step (a-1) is significantly different from hybridization intensity thereof in the step (a-2).

The above screening method enable to select genes wherein expression of the genes may be changed due to difference in culture condition. The “industrial yeast cultured in a different condition” includes yeasts cultured in different medium or temperature, or sequential samples during the process of producing an alcohol or an alcoholic beverage, for example.

Genes obtained by the various screening method of the present invention may be subject for further functional analysis, because differences in hybridization intensity may contribute to the difference of fermentation character between strains. The genes which have nucleotide polymorphism detected by the method mentioned above can be also selected for functional analysis.

As another alternative, the DNA array of the present invention may be used to analyze the chromosomal structure of an industrial yeast by DNA array-based comparative genomic hybridization. Following descriptions are an example of the chromosome structure analysis using the DNA array.

Yeast genomic DNA is prepared using a Quiagen Genomic Tip 100/G (#10243) and Qiagen Genomic DNA Buffer Set (#19060) according to the manual attached to the kit. The DNA (10 μg) is digested with DNase I (manufactured by Invitrogen) according to a method of Winzeler, et al. (Science, volume 281, pages 1194-1197 (1998)), biotinylated using a terminal transferase (manufactured by Roche) and hybridized to a DNA array (Affymetrix Gene Chip Yeast Genome S98 Array). The DNA array of the present invention may include DNAs of any one of (1) to (4) and (7) to (10) from Non-Sc type genes as well as (5) and/or (6) and/or (5′) and/or (6′) of the Sc type. Hybridization and detection of the signal intensity of DNA array are carried out using a Gene Chip Analysis Basic System and analysis soft ware (GCOS; GeneChip Operating Software 1.0) manufactured by Affymetrix.

The signals of probes hybridized with the DNA of brewing yeast were lined following genes order in each chromosome using a spreadsheet program (Microsoft Excel 2000) and the signals are shown in bar graphs. The Sc type genes do not hybridize to the non-Sc type DNA probes (i.e., DNAs of (1) to (4)). Contrary, the non-Sc type genes do not hybridize to the Sc type DNA probes (i.e., DNAs of (5) and/or (6)). Therefore, the gene dosage of Sc type DNA and/or non-Sc type DNA affect the signal and the points where the signals show vigorous changes are considered to be translocation sites between Sc type and non-Sc type chromosome. In a preferred embodiment, hybridization to DNAs of any of (1) to (4) can be determined by hybridization to DNAs of (1) and/or (2), but not to DNAs of (7) and/or (8), and hybridization to DNAs of any of (5) and/or (6) can be determined by hybridization to DNAs of (5) and/or (6), but not to DNAs of (5′) and/or (6′).

### IV. Genes and Polypeptides

The present invention further provides a non-Sc gene obtained by the analyzing method and the screening method of the present invention. Preferably, genes of the present invention participating in increase in productivity and/or improvement in flavor in the production of an alcohol or an alcoholic beverage. For example, genes of the present invention are, preferably, characterized in that concentration of sulfite in a culture medium of an industrial yeast increases when the gene is expressed in the yeast.

The present invention also provides polypeptides or proteins encoded by the gene. The polypeptides of the present invention may be obtained by any known conventional methods including expression of the gene by genetic engineering, or via polypeptide synthesizing technique.

The genes of the present invention may include both ORF and non-coding regions (non-ORF regions). “Gene” includes DNA, cDNA, mRNA, cRNA, without any limitation. The gene of the present invention can be prepared by any known conventional methods, for example, by screening a cDNA library, or via polynucleotide synthesizing techniques. In the present specification, the term “nucleic acid” is use interchangeably with “gene”. In the following explanation, “DNA” will be explained as an example of gene.

With regard to the DNA which can be obtained by the method of the present invention, a DNA containing the DNA sequence of the non-Sc type gene obtained in the above and a DNA which hybridizes to the said DNA under stringent condition and a protein having the substantially the same biological activity may be listed.

The DNA obtained by the method of the present invention includes single-stranded and double-stranded DNAs although they are non-limitative. A DNA which hybridizes to the DNA containing a DNA sequence of the non-Sc type gene obtained in the above under stringent condition includes a degenerated mutant of codon of the protein encoded by the said gene, for example. A degenerated mutant means a polynucleotide fragment encoding the same amino acid sequence by degeneration of codon, although in terms of a DNA sequence, it is different from a DNA sequence of the non-Sc type selected by the present invention.

As an example, the gene of the present invention includes a nucleic acid encoding a polypeptide of any one of the following i) and ii):

i) a polypeptide having the amino acid sequence represented by SEQ ID NO:3; and

ii) a polypaptide having an amino acid sequence wherein one or more amino acid residue(s) is deleted, substituted and/or added to the amino acid sequence represented by SEQ ID NO:3, and having an activity to increase concentration of sulfite in a culture medium of an industrial yeast when the gene is expressed in the yeast.

More preferably, the nucleic acid according to the present invention is selected from the following a) and b):

(a) a nucleic acid having the nucleotide sequence represented by SEQ ID NO:1; and

(b) a nucleic acid having a nucleotide sequence which hybridizes to the nucleotide sequence complementary to the nucleotide sequence represented by SEQ ID NO:1 under a stringent condition, and encodes a polypeptide having an activity to increase concentration of sulfite in a culture medium of an industrial yeast when the gene is expressed in the yeast.

Specific example thereof his a DNA with a sequence as shown by SEQ ID NO:1, a DNA which hybridizes to the said DNA under stringent condition, etc. The DNA which hybridizes under stringent condition means a DNA which is prepared by a colony hybridization method, a plaque hybridization method, a southern blot hybridization method or the like using a DNA fragment with the sequence of the non-Sc type identified hereinabove as a probe. An appropriate stringent condition can be selected as described above.

Hybridization may be carried out according to a method mentioned in “Molecular Cloning, Third Edition”, “Current Protocols in Molecular Biology”, “DNA Cloning 1: Core Techniques, A Practical Approach, Second Edition, Oxford University (1995)”, etc.

Specific examples of the hybridizable DNA is a DNA which shows at least not less than 60% identity, preferably a DNA which shows not less than 80% identity and, more preferably, a DNA which shows not less than 95% identity to a DNA sequence as shown in SEQ ID NO:1 when calculation is conducted using a parameter of the default setting (initial setting) by a software for homology searching such as FASTA, BLAST, Smith-Waterman “Meth. Enzym., volume 164, page 765 (1988)”, etc.

An example of the DNA obtained by the screening method of the present invention is a DNA encoding a polypeptide comprising an amino acid sequence as shown by SEQ ID NO:3, or a DNA which hybridizes to the said DNA under stringent condition.

An example of the polypeptide which is encoded by the DNA obtained by the screening method of the present invention is a polypeptide encoded by the DNA containing the DNA sequence of ORF obtained in the above and a polypeptide encoded by the DNA which is hybridized to the said DNA under stringent condition or a polypeptide comprising an amino acid sequence as shown by SEQ ID NO:3.

Further, a polypeptide having an amino acid sequence wherein one or more amino acid residue(s) is deleted, substituted and/or added to the amino acid sequence of the said polypeptide, and has substantially same biological activity as the activity of the said polypeptide is also included in the present invention. The expression reading “substantially same activity as the activity of the said polypeptide” means the same activity as the activity which is represented by enzymatic activity or the function inherent to the polypeptide before the deficiency, substitution or addition. The said polypeptide can be prepared by a site-specific mutation introduction which is mentioned in “Molecular Cloning, Third Edition”, “Current Protocols in Molecular Biology”, “Nuc. Acids. Res., volume 10, page 6487 (1982)”, “Proc. Natl. Acad. Sic. USA, volume 79, page 6409 (1982)”, “Gene, volume 34, page 315 (1985)”, “Nuc. Acids. Res., volume 13, page 4431 (1985)”, “Proc. Natl. Acad. Sci. USA, volume 82, page 488 (1985)”, etc. For example, it is able to be prepared by introducing a site-specific mutation into a DNA encoding a polypeptide comprising an amino acid sequence as shown in SEQ ID NO:3 or 4. Although there is no particular limitation for the number of the amino acid residue(s) which is/are deficient and/or substituted and/or added, the number is within such an extent that is able to be deficient and/or substituted and/or added by known methods such as the above-mentioned site-specific mutation method and is one to several tens, preferably 1 to 20, more preferably 1 to 10 and, still more preferably, 1 to 5.

A polypeptide having an amino acid sequence wherein one or more amino acid residue(s) is deleted, substituted and/or added to the amino acid of the polypeptide of the present invention means that there is/are one or more deficiency (ies) and/or substitution(s) and/or addition(s) of one or more amino acid residue(s) in any one or more position(s) of the amino acid sequence in the same sequence. Those deficiency (ies) and/or substitution(s) and/or addition(s) may take place at the same time and the substituted or added amino acid residue may be either naturally occurring type or a non-naturally occurring type. Examples of the amino acid residue of a natural type are L-alanine, L-asparagine, L-aspartic acid, L-glutamine, L-glutamic acid, glycine, L-histidine, L-isoleucine, L-leucine, L-lysine, L-methionine, L-phenylalanine, L-proline, L-serine, L-threonine, L-tryptophan, L-tyrosine, L-valine and L-cysteine, etc.

Examples of the amino acid residue which is able to be substituted each other will be shown below. Amino acid residues in the same group may be substituted each other (conservative substitution).

Group A: leucine, isoleucine, norleucine, valine, norvaline, alanine, 2-aminobutanoic acid, methionine, O-methylserine, tert-butylglycine, tert-butylalanine and cyclohexylalanine.

Group B: aspartic acid, glutamic acid, isoaspartic acid, isoglutamic acid, 2-aminoadipic acid and 2-aminosuberic acid.

Group C: asparagine and glutamine.

Group D: lysine, arginine, ornithine, 2,4-diaminobutanoic acid and 2,3-diaminopropionic acid.

Group E: proline, 3-hydroxyproline and 4-hydroxyproline.

Group F: serine, threonine and homoserine.

Group G: phenyl alanine and tyrosine.

For the purpose that the resulting mutated polypeptide has the substantially same activity as the activity of the polypeptide before the mutation, it is preferred that the mutated one has at least 60% or more, usually 80% or more or, particularly, 95% or more of identity to the amino acid sequence of the polypeptide before the mutation when calculation is carried out using a parameter of the default setting (initial setting) by a software for the analysis such as BLAST and FASTA. Identity between two amino acid sequence can be determined as discussed above.

It is also possible to produce the polypeptide of the present invention by a chemical synthetic method such as Fmoc method (fluorenylmethyloxycarbonyl method), tBoc method (tert-butyloxycarbonyl method), etc. It is further possible to chemically synthesize by using a peptide synthesizers manufactured by Advanced ChemTech, Perkin-Elmer, Pharmacia, Protein Technology Instrument, Synthecell-Vega, PerSeptive, Shimadzu, etc.

When the method of the present invention is used, it is possible to determine the whole genome sequence of industrial yeast, to identify the useful genes of industrial yeast and to assign the functions of the said genes. There are many cases where the genes in industrial yeast are industrially useful and, when the genes are classified on the basis of the assigned functions, character of the yeast is clarified and precious information for breeding of industrial yeast is able to be obtained. For example, when the industrial yeast is a brewing yeast, then a gene participating in increase in productivity and improvement in flavor in the production of alcoholic beverage is identified and, in case the gene is disadvantageous for the increase of productivity or for the improvement of flavor, the gene expression is suppressed by a gene disruption or an antisense method or an RNAi method (c.f., for example, Non-Patent Document 10), whereupon yeast which shows an excellent brewing character can be bred. In case the gene is advantageous for the increase of productivity, improvement of flavor, etc., then for example the gene is overexpressed in the yeast, whereupon brewing yeast which shows an excellent brewing character, which is industrially useful, can be bred.

An example where the gene obtained by the method of the present invention is used to breed useful yeast is shown as follows.

As already mentioned above, when the sulfite concentration in a product is increased, it is possible to make a product with excellent flavor stability. Therefore, if the gene obtained by the screening method of the present invention contributes to production and efflux of sulfite, it is now possible that a transformant is cultivated and expressed the said gene to make a product with excellent flavor stability as a result of the increase in the concentration of sulfite in the product.

It has been known that a bottom fermenting yeast reduces sulfate ion (SO₄²⁻) taken from outside of the cell to sulfite ion (SO₃²⁻). However, sulfite inhibits glyceroaldehyde-3-phosphate dehydrogenase and reduces the concentration of intracellular ATP, therefore, yeast has a function of discharging sulfite so that excessive sulfite should not be accumulated in the cell. SSU1 is a gene, which has been isolated and shown complement the sulfite-sensitive mutation (c.f., for example, Non-Patent Document 11). SSU1 gene product comprises 485 amino acid residues, and the structural analysis suggests that it is a transporter with 9 to 10 membrane-spanning domains (c.f., for example, Non-Patent Document 12). Further, as a result of experiment using a SSU1 overexpressed strain, it has been already proved that the SSU1 gene product participates in discharge of sulfite (c.f., for example, Non-Patent Document 13).

Bottom fermenting yeast usually has a high production ability of sulfite, while top fermenting yeast rarely produces it. By using a screening method of the present invention, it is possible to select non-ScSSU1 gene which is specific to bottom fermenting yeast in addition to ScSSU1 gene which exists in both top and bottom fermenting yeast.

Breeding method of yeast, wherein the non-ScSSU1 gene 4 is intensified, is specifically mentioned in the Examples.

### V. Recombinant Vectors, Transformants and Other Embodiments

The present invention further includes the following embodiments:

A recombinant vector containing the gene of the present invention; A transformant comprising the recombinant vector. The transformant is preferably, a yeast of genus Saccharomyces

A method for producing an alcohol or an alcoholic beverage using the transformant of the present invention. Preferably, the present method may comprise culturing the transformant of the present invention in a sugar-containing medium. Preferably, the sugar containing medium can be selected from a group consisting of wort, grape juice, rice juice and glucose and/or maltose syrup.

A breeding method of yeast which is suitable for the production of an alcohol or an alcoholic beverage, characterized in that, expression of the gene or the nucleic acid of the present invention is controlled. Preferably, the yeast belongs to the genus Saccharomyces.

Yeast obtained by the breeding method of the present invention. A method for producing an alcohol or an alcoholic beverage using the yeast of the present invention.

An alcohol or an alcoholic beverage which is produced by the producing method of the present invention.

With regard to yeast used as a host in the introduction of gene selected by the screening method of the present invention, there is no particular limitation so far as it is yeast which is usable for brewing, and any yeast which is widely used as a brewing yeast at present such as beer yeast including NCYC456, NBRC 1951, NBRC 1952, NBRC 1953 and NBRC 1954 may be used. Further, whisky yeasts (such as S. cerevisiae NCYC 90), wine yeasts (such as wine yeast Kyokai No. 1, No. 3, No. 4, etc.) and sake yeasts (such as sake yeast Kyokai No. 7, No. 9, etc.) may be also used.

With regard to a vector used for the introduction of gene into the above-mentioned host, there is no particular limitation so far as it is a vector which can express gene in the yeast, and any of plasmid of a multicopy (YEp type), a single-copy plasmid (YCp type) and a chromosomal DNA-integrating plasmid (YIp type) may be utilized. An example of a YEp vector is YEp 51 (J. R. Broach, et al., Experimental Manipulation of Gene Expression, Academic Press, New York, 83, 1983), etc. an example of a YCp vector is YCp 50 (M. D. Rose, et al., Gene, volume 60, page 237, 1987), etc.; and an example of a YIp vector is YIp 5 (K. Struhl, et al., Proc. Natl. Acad. Sci. USA, volume 76, page 1035, 1979), etc. Those plasmids are put into the market and are easily available.

The above-mentioned vector may have other sequence for controlling expression of gene in yeast such as, promoter, operator, enhancer, silencer, ribosome binding sequence, terminator, etc. With regard to a promoter and a terminator for a constitutive expression of a gene, there is no particular limitation but any combination may be used so far as it functions in a brewing yeast and is independent from sulfite concentration in the product. As to a promoter for example, it is possible to use a promoter for glyceraldehyde-3-phosphate dehydrogenase (TDH3) gene, a promoter for phosphoglycerate kinase (PGK1) gene, etc. Those promoters have been known, and PGK1 gene, for example, is mentioned in detail in publicly known documents such as M. F. Tuite, et al., EMBO J., volume 1, page 603 (1982) and is easily available.

It is not necessary that the above-mentioned other sequences which regulate the expression of the introduced gene are particularly provided from vector so far as the DNA obtained by the screening method of the present invention includes them. When such other sequences are not contained in the said DNA, it is preferred that other sequences are prepared separately and ligated to the said DNA. Alternatively, even in the case of higher expression level or specific regulation of expression is required, other sequences appropriate for such a purpose are ligated to the said DNA.

A method for the transformation of the above vector to a host may follow known procedures. For example, the following methods may be used; an electroporation method “Meth. Enzym., volume 194, page 182 (1991)”, a spheroplast method “Proc. Natl. Acad. Sci. USA, volume 75, page 1929 (1978)”, a lithium acetate method “J. Bacteriology, volume 153, page 163 (1983)”, a method mentioned in “Proc. Natl. Acad. Sci. USA, volume 75, page 1929 (1978)”, etc.

To be more specific, a host is cultivated in a standard yeast nutrient medium (such as YEPD medium “Genetic Engineering, vol. 1, Plenum Press, New York, 117 (1979)”, etc.) so that the absorbance at 600 nm becomes 1 to 6. Cells are collected by centrifugation, washed and subjected to a pre-treatment with an alkali metal ion or, preferably, lithium ion in a concentration of about 1M to 2M. After the cells are incubated at about 30° C. for about 60 minutes, they are incubated together with DNA to be introduced (about 1 to 20 μg) at about 30° C. for about 60 minutes. Polyethyleneglycol or, preferably, polyethyleneglycol of about 4,000 daltons is added as the final concentration will be about 20% to 50%. After the incubation is carried out at about 30° C. for about 30 minutes, the cells are subjected to a heating treatment at about 42° C. for about 5 minutes. Preferably, the cell suspension is washed with a standard yeast nutrient medium and placed in a predetermined amount of a fresh standard yeast nutrient medium, then incubated at about 30° C. for about 1 hour. After the incubation, it is spread on an appropriate selective medium plate.

Besides the above, as for a general cloning technique, “Molecular Cloning, Third Edition”, “Methods in Yeast Genetics, A Laboratory Manual (Cold Spring Harbor Laboratory Press, Cold Spring Harbor, N.Y.)”, etc. were referred to.

With regard to a selective marker used for the transformation, it is not possible to utilize an auxotrophic marker in the case of brewing yeast and, therefore, G 418-resistance gene (G 418^{r}), copper-resistance gene (CUP 1) “M. Karin, et al., Proc. Natl. Acad. Sci. USA, volume 81, page 337, 1984”, serulenin-resistance gene (fas2m, PDR 4) (“Atsushi Inogoshi, et al., Seikagaku, volume 64, page 660, 1992”, “M. Hussain, et al., Gene, volume 101, page 149, 1991”, etc. are applicable.

The brewing yeast bred according to the present invention is not different from a parental strain in terms of growth and fermentation ability of yeast. Accordingly, materials, facilities for the production, production control, etc. may be entirely the same as those in the conventional methods, which is an important aspect of the present invention. However, it goes without saying that, conditions such as fermentation period may be changed on a case-by-case, if desired. For example, when a brewing yeast in which discharging ability of sulfite is intensified and an alcoholic beverage is produced using such yeast, only the content of sulfite in the product changes, and there is no difference from the case where a parental strain is used, in terms of growth and fermentation ability of the yeast. Accordingly, materials, facilities for the production, production control, etc. may be entirely the same as those in the conventional methods, and there is no increase in the cost of production of an alcoholic beverage in which sulfite content increases and of which flavor is improved.

### EXAMPLES

Details of the present invention are mentioned with the following Examples although the present invention is not limited to the following Examples.

### Example 1

### Preparation of Chromosomal DNA of Saccharomyces pastorianus Weihenstephan 34/70 (hereinafter, Abbreviated as Strain 34/70)

Preparation of chromosomal DNA was carried out by a method mentioned in “Yeast, a practical approach (IRL Press) 6.2.1 (pages 228-229)”, which was partially modified. Cells were inoculated and grown in 200 mL of YPD medium (2% glucose, 1% yeast extract and 2% polypeptone) at 30° C. until absorbance of the culture at 660 nm became 4. Cells were collected by centrifugation, washed with buffer A (50 mM sodium phosphate, 25 mM EDTA and 1% (v/v) β-mercaptoethanol; pH 7.5) and resuspended in 25 mL of buffer A 7 mg of Zymolyase 100T (Seikagaku Kogyo) was added thereto and the mixture was mildly shaken at 37° C. for 60 minutes. To this was added 25 mL of buffer B (0.2M Tris-HCl, 80 mM EDTA and 1% SDS; pH 9.5), then the mixture was allowed to stand at 65° C. for 30 minutes, cooled on ice, mixed with 12 mL of 5M potassium acetate and allowed to stand on ice for further 60 minutes. The resulting solution was centrifuged at 5,000 g for 10 minutes at 15° C. To the recovered supernatant was added the same volume of ethanol to precipitate DNA, and the mixture was immediately centrifuged at 5,000 g for 10 minutes at 15° C. to collect the precipitate. The resulting precipitate was washed with 70% (v/v) ethanol, subjected to natural drying and dissolved in 5 mL of TE buffer (10 mM Tris-HCl and 1 mM of EDTA; pH 8.0) to give a crude DNA solution. Cesium chloride (4.06 g) and 840 μg of bisbenzimide (Hoechst 33258) were added and dissolved in 3.5 mL of the crude DNA solution, the mixture was subjected to centrifugal separation at 100,000 g for 17 hours at 25° C. and exposed to, UV light to make DNA bands visible, whereupon the band of the lower layer was recovered. The recovered DNA solution was extracted with isopropanol which was saturated with a cesium chloride solution to remove bisbenzimide (Hoechst 33258). To the recovered aqueous layer was added 4-fold by volume of 0.3 M sodium acetate followed by mixing. Then, 3-fold by volume of ethanol was added thereto to precipitate the DNA, which was recovered by centrifugation. The recovered DNA was dissolved in TE buffer containing 75 μg/mL of RNase, kept at 37° C. for 5 minutes, and extracted with phenol/chloroform for three times and the aqueous layer was further subjected to precipitation with ethanol. The precipitate recovered by centrifugation was washed with 70% (v/v) ethanol, subjected to natural drying and dissolved in TE buffer to prepare a chromosomal DNA solution.

### Example 2

### Preparation of a Shotgun Library

The concentration of a genome solution of strain 34/70 prepared in Example 1 was adjusted to 1 mg/mL using a TE buffer. 0.1 mL thereof was treated with a Hydroshear (manufactured by GeneMachines; speed: 6; cycle: 20) to fragmentize the genomic DNA. End of the genomic fragment was blunted using a DNA Blunting Kit (manufactured by Takara Shuzo), fractionated by 0.8% agarose electrophoresis. Genomic fragments of 1.5 to 2.5 kb were excised from the gel, and DNA was eluted. The DNA eluate was treated with phenol/chloroform and precipitated with ethanol to give a genome library insert. All the above insert and 0.5 μg of pUC 18 SmaI/BAP (manufactured by Amersham Biosciences) were subjected to ligation at 15° C. for 15 hours using T4 ligase (manufactured by Takara Shuzo).

The ligation reaction product was precipitated with ethanol and dissolved in 10 μL of a TE buffer. A ligation solution (1 μL) was inserted into 40 μL of Escherichia coli Electro Cell DH5α (manufactured by Takara Shuzo) by means of electroporation under the condition mentioned in the attached experimental manual. The resulting product is spread on an LB plate medium containing 1.6% of agar (the LB medium (1% bactotryptone, 0.5% yeast extract and 1% sodium chloride; pH 7.0)) containing 0.1 mg/mL of ampicillin; 0.1 mg/mL of X-gal and 1 mmol/L of isopropyl-β-D-thiogalactopyranoside (IPTG), and incubated through the night at 37° C.

The transformant obtained from colonies formed on the said plate medium was subjected to cultivation without shaking through the night at 37° C. in a 384-well titer plate to which 50 μL of an LB medium containing 0.1 mg/mL of ampicillin was added. Then 50 μL of a 50% aqueous solution of glycerol was added thereto followed by stirring, and the mixture was used as a glycerol stock.

### Example 3

### Preparation of a Cosmid Library

About 0.1 mg of the genomic DNA obtained in Example 1 was partially digested with Sau3AI (manufactured by Takara Shuzo). Insertion of the fragment into a BamHI site of Super Cos I vector (manufactured by Stratagene) was carried out according to a manual. A ligated product prepared by this method was subjected to packaging using Gigapack III Gold (manufactured by Stratagene) and introduced into Escherichia coli XL1-Blue MR strain (manufactured by Stratagene) according to a manual. It was spread on an LB plate medium containing 0.1 mg/mL of ampicillin and incubated through the night at 37° C. The resulting transformants were cultured through the night at 37° C. in an LB medium (each well: 50 μL) containing 0.1 mg/mL of ampicillin using a 96-well titer plate, and then 50 μL of 50% glycerol solution was added thereto followed by stirring and the mixture was used as a glycerol stock.

### Example 4

### Determination of DNA Sequence

(4-1) Preparation of DNA Fragment

The whole genome sequence of strain 34/70 was determined mainly using the whole genome shotgun method. A DNA fragment of which DNA sequence is to be determined by that method was prepared by a PCR method from the shotgun library prepared in the above Example 2. To be specific, clone derived from the whole genome shotgun library was inoculated using a replicator (manufactured by Gene Solution) to a 384-well titer plate where 50 μL of an LB medium containing 0.1 mg/mL of ampicillin was placed to each well and cultivated without shaking through the night at 37° C. The culture liquid was transferred to a 384-well reaction plate (manufactured by AB Gene) containing 10 μL of reaction mixture for PCR (TaKaRa Ex Taq manufactured by Takara Shuzo) using a replicator (manufactured by Gene Solution) and PCR was carried out according to a protocol by Makino, et al. “DNA Research, volume 5, pages 1 to 9 (1998)” using a GeneAmp PCR System 9700 (manufactured by Applied Biosystems) to amplify the inserted fragment. After that, excessive primers and nucleotides were removed by a PCR product purification kit (manufactured by Amersham Bioscience) and a sequence reaction was carried out using that sample as a template.

A DNA fragment from the cosmid library of the above Example 3 was prepared according to the following method. That is, clone derived from the whole cosmid library was inoculated to each well of a 96-well plate to which 1.0 mL each of a 2×YT medium (1.6% bactotryptone, 0.1% yeast extract and 0.5% sodium chloride; pH 7.0) containing 50 μg/mL of ampicillin was placed and subjected to shake culture at 30° C. through the night. A cosmid DNA was prepared from the said culture using KURABO PI-1100 AUTOMATIC DNA ISOLATION SYSTEM (manufactured by KURABO) according to a manual of KURABO, and was used as a template for a sequence reaction.

(4-2) Sequence Reaction

A sequence reaction mixture was prepared as follows. The PCR product or cosmid DNA prepared in the above (4-1) is mixed with about 2 μl of DYEnamic ET Terminator Sequencing Kit (manufactured by Amersham Bioscience) and appropriate primers to give about 8 μl of reaction mixture. An M13 forward (M13-21) primer and an M13 reverse (M13RV) primer (manufactured by Takara Bio), are used for the sequence reaction of a PCR product derived from shotgun DNA, while a forward primer SS-cos F.1 (SEQ ID NO: 7) and a reverse primer SS-cos R.1 (SEQ ID NO: 8) are used for cosmid DNA. Amounts of the primers and the DNA fragment were 3.2 pmol and 50 to 200 ng, respectively. The said reaction solution was subjected to dye terminator sequence reaction of 60 cycles using a GeneAmp PCR System 9700. Cycle parameter followed a manual attached to the DYEnamic ET.Terminator Sequencing Kit. Purification of the sample was carried out using a Multi Screen HV Plate (manufactured by Millipore) according to a manual of Millipore. The purified reactant was stored in a dark place of 4° C. The purified reactant was analyzed using a Mega BACE 1000 Sequencing System (manufactured by Amersham Bioscience) and ABI PRISM 3700 DNA Analyser (manufactured by Applied Biosystems) according to manuals attached thereto. The data on 332,592 sequences obtained by the Mega BACE 1000 Sequencing System and on 13,461 sequences obtained by the 3700 DNA Analyser were transferred to a server Enterprise 6500 (manufactured by Sun Microsystems) and preserved. The data on 346,053 sequences corresponded to about 7-fold of the genome size.

A list of the primers for the PCR used in the Example is shown in Table 3.

### Example 5

### Assembly (A Process whereby the Order of Multiple Sequenced DNA Fragments is Determined)

All works for reconstruction of genomic DNA sequence from sequence information for DNA fragment of the 346,053 sequences obtained in the above Example 4 were carried out on a UNIX® platform. Base call was carried out by phred (The University of Washington), masking of vector sequence was carried out using Cross_Match (The University of Washington) and assembly was carried out using Phrap (The University of Washington). Contig obtained as a result of the assembly was analyzed using a graphical editor consed (The University of Washington). A series of works from base call to assembly was carried out all together utilizing a script phredphrap attached to the consed.

### Example 6

### Preparation of a Comparative Database with the Whole Genome Sequence of S. cerevisiae

S. pastorianus is believed to be a natural hybrid of S. cerevisiae with its closely-related species “Int. J. Syst. Bacteriol., volume 35, pages 508 to 511 (1985)”. Therefore, a DNA sequence (comprising 10,044 bases) of both ends of the cosmid DNA clone obtained in (4-2) was subjected to a homology searching by a homology searching algorithm to S. cerevisiae genome sequence, whereupon for each DNA sequence alignment of homologous region on S. cerevisiae genome sequence and the identity thereof were determined to prepare a database. An identity distribution chart for cosmid DNA sequence with the corresponding genomic DNA sequence of S. cerevisiae is shown in FIG. 2. The DNA sequence of cosmid was roughly classified into a DNA sequence group showing not less than 94% identity to the genomic DNA sequence of S. cerevisiae and a DNA sequence group showing around 84% identity thereto. The DNA sequence group showing not less than 94% identity is named a DNA sequence of an Sc type derived from S. cerevisiae and the DNA sequence group showing around 84% identity is named a DNA sequence of a non-Sc type derived from genome of closely related species. Similarly, a comparative database (Table 1) was prepared for the DNA sequence of both ends of shotgun clone obtained in (4-1) with the genomic DNA sequence of S. cerevisiae. Table 1 shows an example of the comparative database of DNA sequence of both ends of 3,648-cosmid clone with the genomic DNA sequence of S. cerevisiae. There are shown the homologous region and the identity of forward sequence and reverse sequence of cosmid subjected to the DNA sequence determination on each genomic DNA sequence of S. cerevisiae.

TABLE 1

| | Forward Chain | | | | | | Reverse Chain | | | | | |
|---|---|---|---|---|---|---|---|---|---|---|---|---|
| | | Matched S. cerevisiae Genomic Base Sequence | | | | | | Matched S. cerevisiae Genomic Base Sequence | | | | |
| | | Information | | | | | | Information | | | | |
| | | | Chro- | | | | | | Chro- | | | |
| | Sequence | Identical | mo- | Initiation | Termination | | Sequence | Identical | mo- | Initiation | Termination | |
| Name of | Length | Length | some | Position | Position | Identity | Length | Length | some | Position | Position | Identity |
| Cosmid | (bases) | (bases) | No. | (bases) | (bases) | (%) | (bases) | (bases) | No. | (bases) | (bases) | (%) |
| SSL052_A06 | 627 | 625 | XVI | 15,940 | 16,565 | 98.7 | 626 | 625 | XVI | 52,979 | 52,354 | 98.7 |
| SSL023_D02 | 346 | 341 | XVI | 16,784 | 17,125 | 87.3 | 633 | 629 | XVI | 66,017 | 65,388 | 90.5 |
| SSL015_E09 | 630 | 625 | XVI | 39,030 | 39,655 | 89.5 | 615 | 614 | XVI | 81,655 | 81,041 | 97.9 |
| SSL029_B08 | 664 | 660 | XVI | 45,916 | 45,256 | 99.3 | 650 | 647 | XVI | 8,504 | 9,151 | 98.8 |
| SSL028_G10 | 656 | 655 | XVI | 47,609 | 46,954 | 98.3 | 646 | 641 | XVI | 10,359 | 11,000 | 98.0 |
| SSL008_E01 | 622 | 620 | XVI | 46,362 | 46,982 | 93.4 | 589 | 587 | XVI | 86,022 | 85,435 | 98.3 |
| SSL030_G05 | 632 | 631 | XVI | 47,013 | 47,644 | 99.2 | 618 | 617 | XVI | 87,004 | 86,387 | 99.5 |
| SSL032_H10 | 646 | 645 | XVI | 52,076 | 51,431 | 98.1 | 637 | 636 | XVI | 13,273 | 13,909 | 98.7 |
| SSL041_G05 | 635 | 634 | XVI | 52,979 | 52,345 | 99.4 | 619 | 618 | XVI | 9,825 | 10,443 | 99.4 |
| SSL031_D08 | 659 | 658 | XVI | 52,297 | 52,955 | 99.2 | 638 | 637 | XVI | 92,295 | 91,658 | 99.1 |
| SSL069_F11 | 417 | 414 | XVI | 55,053 | 55,467 | 88.5 | 788 | 787 | XVI | 97,115 | 96,328 | 94.4 |
| SSL005_A10 | 647 | 645 | XVI | 65,233 | 64,588 | 99.2 | 527 | 516 | XVI | 21,537 | 22,053 | 81.8 |
| SSL014_G07 | 628 | 627 | XVI | 65,229 | 65,856 | 99.8 | 621 | 620 | XVI | 103,674 | 103,054 | 99.2 |

On the basis of the information obtained by the prepared comparative database, mapping of cosmid clone and shotgun clone on S. cerevisiae genome sequence was carried out (FIG. 3). In addition, a comparative database (Table 1) of contig DNA sequence obtained in Example 5 with S. cerevisiae genome sequence was prepared, then mapping was carried out. Although the means for the mapping was almost the same as the above-mentioned method, if forward and reverse sequences of cosmid and shotgun clones were present in different contigs, these contigs were connected by forward-reverse link (FIG. 4).

### Example 7

### Identification and Assignment of Function of ORF

Identification of ORF (open reading frame) in the DNA sequence assembled in Example 5 was carried out. The examples are specifically shown below. Identification of ORF existing in the DNA sequence assembled in Example 5 was carried out using a available program using ORF finder (http://www.ncbi.nih.gov/gorf/gorf.html) for identification of ORF for six kinds of reading frames in the sequence with the length of not less than 150 bases from initiation codon to termination codon including its complementary sequence. Assignment of function of the extracted ORF was carried out by homology searching of amino acid sequence of ORF of S. cerevisiae that has been registered at the SGD and published. Table 2 is a table showing examples of the ORF name of S. cerevisiae corresponding to the result of assignment of function of ORF existing in the non-Sc genome. From the left side of the table, there are shown name of the ORF existing on the brewing yeast, ORF length in polynucleotide, ORF length in polypeptide, name of the ORF of S. cerevisiae determined by homology searching, identity, coincided length and functions of the gene.

TABLE 2

| | ORF | ORF | Name of | | Coincided | |
|---|---|---|---|---|---|---|
| | Length | Length | Homologous | Identity | Length | |
| Name of ORF | (bp) | (aa) | Gene | (%) | (aa) | Functions |
| nonSc-ATF2 | 1638 | 545 | ATF2 | 71 | 535 | alchhol O-acetyltransferase |
| nonSc-THI3 | 1305 | 434 | THI3 | 94 | 431 | transcriptional activator |
| nonSc-FUS3 | 435 | 144 | FUS3 | 90 | 139 | MAP kinase |
| nonSc-ILV5 | 1188 | 395 | ILV5 | 97 | 395 | ketol-acid reductoisomerase |
| nonSc-MET2 | 1461 | 486 | MET2 | 93 | 486 | homoserine O-acetyltransferase |
| nonSc-MET10 | 3108 | 1035 | MET10 | 87 | 1035 | sulfite reductase (NADPH) |
| nonSc-MET14 | 609 | 202 | MET14 | 97 | 202 | adenylsulfate kinase |
| nonSc-MET16 | 786 | 261 | MET16 | 92 | 261 | phosphoadenylyl-sulfate reductase |
| nonSc-TPI1 | 747 | 248 | TPI1 | 96 | 248 | triosephosphate isomerase |
| nonSc-MET3 | 1536 | 511 | MET3 | 94 | 511 | sulfate adenylyltransferase (ATP) |
| nonSc-MET10 | 3108 | 1035 | MET10 | 87 | 1035 | sulfite reductase (NADPH) |
| nonSc-SAM1 | 1149 | 382 | SAM1 | 97 | 382 | methionine adenosyltransferase |
| nonSc-SSU1 | 1377 | 458 | SSU1 | 78 | 457 | sulfite transporter |

### Example 8

### Analysis of Chromosome Structure by DNA Array-Based Comparative Genomic Hybridization and PCR

Preparation of genomic DNA from yeast was carried out using Qiagen Genomic Tip 100/G (#10243: manufactured by Qiagen) and Qiagen Genomic DNA Buffer Set (#19060: manufactured by Qiagen) according to-the manuals attached to the kits. The DNA (10 μg) was digested with DNase I (manufactured by Invitrogen) according to a method of Winzeler,. et al. “Science, volume 281, pages 1194 to 1197 (1998)”, biotinylated by a terminal transferase (manufactured by Roche) and hybridized to a DNA array (Affymetrix Gene Chip Yeast Genome S98 Array: produced by Affymetrix). Hybridization and detection of signal intensity of array were carried out using a Gene Chip Analysis Basic System manufactured by Affymetrix.

The signal of each probe hybridized with the DNA of strain 34/70 is normalized to that of the haploid laboratory yeast strain S288C using an analysis soft ware (Microarray Suite 5.0: manufactured by Affymetrix) and shown as signal log ratio (2ⁿ). Signal log ratios were lined following gene order in each chromosome using a spreadsheet program (Microsoft Excel 2000) and the signal log ratios are shown in bar graphs as shown in FIG. 5. The non-Sc type genes do not hybridize to the S. cerevisiae array, therefore, the Sc type gene dosage affect the signal log ratio and the points where the signal log ratios show vigorous changes are considered to be translocation sites between Sc type and non-Sc type chromosome.

On the basis of genome sequence of strain 34/70 determined by a shotgun method, the chimera chromosome structure was confirmed by PCR where two pairs of primers having DNA sequences in which one side is Sc type while the other side is a non-Sc type (XVI-1(L)cer-95894 (SEQ ID NO: 9)/XVI-1(R)nonSc-106302rv (SEQ ID NO: 10) and XVI-2(L)cer-859737 (SEQ ID NO: 11)/XVI-2(R)nonSc-864595rv (SEQ ID NO: 12) were designed and the genomic DNA derived from strain 34/70 was used as a template. Two examples of translocation of chromosome XVI are shown as follows.

The PCR was carried out using Takara LA Taq™ and a buffer attached thereto in accordance with the attached manual by a Takara PCR Thermal Cycler SP.

As a result of the PCR, it was confirmed by a 0.8% agarose electrophoresis that, a DNA fragment in the predicted length was amplified from strain 34/70, while when genomic DNA of the experimental strain S. cerevisiae X2180-1A was used as a template for the PCR, amplification. of the DNA fragment was not detected. Furthermore, when DNA sequence of both ends of the DNA fragment amplified from strain 34/70 was confirmed, it was consistent with the genome sequence determined by a shotgun method and it was confirmed that, within such a region, translocation between Sc type and non-Sc type chromosome took place.

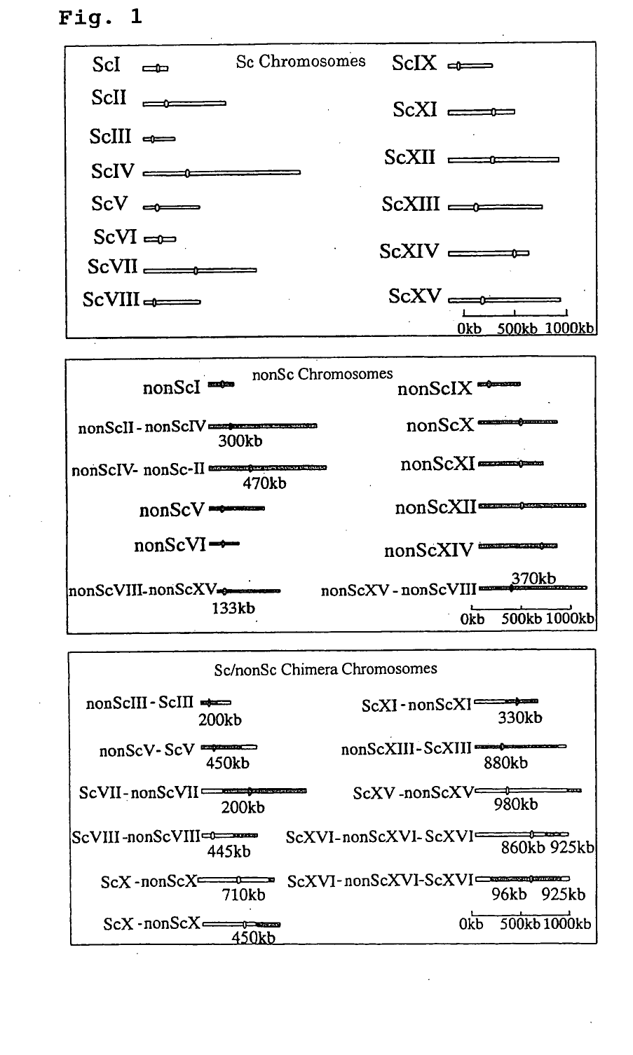

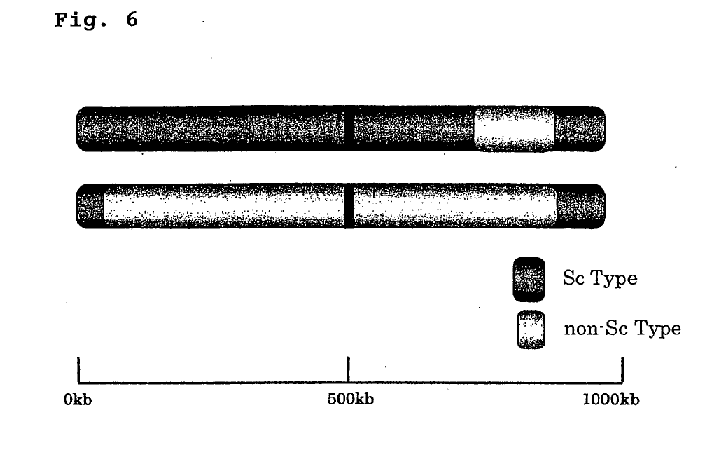

From the above result, it is estimated that at least two kinds of chromosomes were present in the chromosomeXVI as shown in FIG. 6. According to the same techniques, ligation between Sc chromosome and non-Sc chromosome (or inverse thereof) or, in other words, the region where the existence of chimera chromosome structure was suggested was confirmed. Such chimera chromosome structure of the Sc chromosome and non-Sc chromosome was confirmed at least 13 places in the total chromosomes of strain 34/70 (FIG. 1).

As a result of genome analysis, it was found that chromosome structure of bottom fermenting yeast was very complicated and there were at least 37 kinds of chromosomes in strain 34/70.

### Example 9

### Cloning of SSU1 Genes of Strain 34/70

The shotgun clone containing non-ScSSU1 gene was retrieved using a comparative database obtained in Example 6. There was SSS103_G08 which comprises about 2.4 kb of fragment containing full-length of non-ScSSU1 ORF, where identity of forward and reverse sequence of shotgun clone to those of S. cerevisiae were 62.9% and 82.9%, respectively.

SSS103-G08 was selected from a library of genomic DNA, then full length of non-ScSSU1 was prepared by PCR. Synthetic DNAs of SacI-non-Sc-SSU1_for1 (SEQ ID NO: 13) and Bg1II-non-Sc-SSU1_rv1460 (SEQ ID NO: 14) were used as primers. As a result of such a combination, base numbers 1 to 1460 of nonScSSU1 was amplified to give a SacI-Bg1II fragment of about 1.5 kb.

With regard to an ScSSU1 gene, full length of gene was obtained by PCR using a primer pair designed on the basis of the information of SGD using a genomic DNA of strain 34/70 as a template. Synthetic DNAs of SacI-ScSSU1_for1 (SEQ ID NO: 15) and Bg1II-ScSSU1_rv1406 (SEQ ID NO: 16) were used as primers. As a result of such a combination, base numbers 1 to 1406 of ScSSU1 gene was amplified to give a SacI-Bg1II fragment of about 1.4 kb.

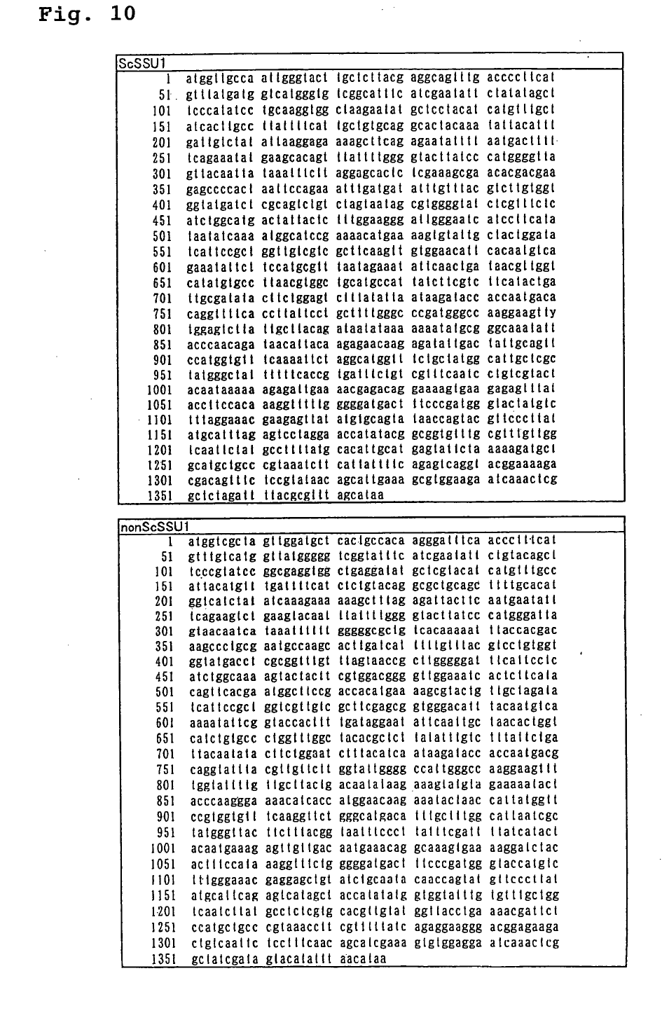

ScSSU1 and non-ScSSU1 gene obtained as above were inserted using TA cloning kit (Invitrogen) into pCR 2.1-TOPO vector attached to the kit, and they were named TOPO/ScSSU1 and TOPO/non-ScSSU1, respectively. Sequences of the resulting ScSSU1 and non-ScSSU1 genes were confirmed by a method of Sanger “F. Sanger, Science, volume 214, page 1215, 1981” (FIG. 10).

### Example 10

### Disruption of each SSU1 Gene

According to a method mentioned in the document “Goldstein, et al., Yeast, volume 15, page 1541 (1999)”, DNA fragment for gene disruption was prepared by PCR using a plasmid containing a drug-resistance marker (pFA6a (G418^{r}), pAG 25 (nat1)) as a template. As primers for the PCR, non-Sc-SSU1_for (SEQ ID NO: 17)/non-Sc-SSU1_rv (SEQ ID NO: 18) were used for disruption of non-ScSSU1 gene, while for disruption of ScSSU1 gene, ScSSU1_for (SEQ ID NO: 19)/ScSSU1_rv (SEQ ID NO: 20) were used. For disruption of non-ScSSU1 gene, a plasmid pPGAPAUR (AUR1-C) and primers non-Sc-SSU1_for+PGAPAUR (SEQ ID NO: 21)/non-Sc-SSU1-rv+AURI-C (SEQ ID NO: 22) were further used. As such, two and three kinds of DNA fragments were prepared for ScSSU1 and non-ScSSU1 gene disruption, respectively.

The bottom fermenting yeast BH 96 was transformed using the DNA fragments for gene disruption prepared with above method. The transformation was carried out by a method mentioned in the Japanese Patent Laid-Open Gazette No. H07/303,475 and concentrations of the drugs were 300 mg/L for geneticin, 50 mg/L for nourseothricin and 1 mg/L for aureobasidin A.

With regard to the transformants prepared, gene disruption was confirmed by Southern analysis. Firstly, the genomic DNA extracted from parental strain and disruptant was subjected to restriction enzyme treatment (at 37° C. or 18 hours) using NcoI for the confirmation of ScSSU1 gene disruption and HindIII for the confirmation of non-ScSSU1 gene disruption, and then fractionated by 1.5% agarose gel electrophoresis and transferred to a membrane. After that, hybridization was carried out (at 55° C. for 18 hours) with a probe specific to the ScSSU1 or non-ScSSU1 following a protocol of the Alkphos Direct Labelling Reagents (Amersham) and signals were detected by CDP-Star.

Each of the strains where gene disruption was confirmed was named as follows.
Sc-1 (ScSSU1/Scssu1::G418^{r})
Sc-2 (Scssu1::G418^{r}/Scssu1::natl)
non-Sc-1 (non-ScSSU1/non-ScSSU1/non-Scssu1::G418^{r})
non-Sc-2 (non-ScSSU1/non-Scssu1: :G418^{r}/non-Scssu1: natl)
non-Sc-3 (non-Scssu1::G418^{r}/non-Scssu1::nat1/non-Scssu1::AUR1-C)

### Example 11

### Quantitative Analysis of Sulfite Production in a Fermentation Test

Fermentation test using parental strain and disruptant Sc-1 to non-Sc-3 prepared in Example 10 was carried out under the following condition.

Original extract: 12.75%

Fermentation scale: 2 liters

Dissolved oxygen concentration: about 9 ppm

Fermentation temperature: 15° C.

Pitching rate: 10 g of wet yeast cells/2 L of wort

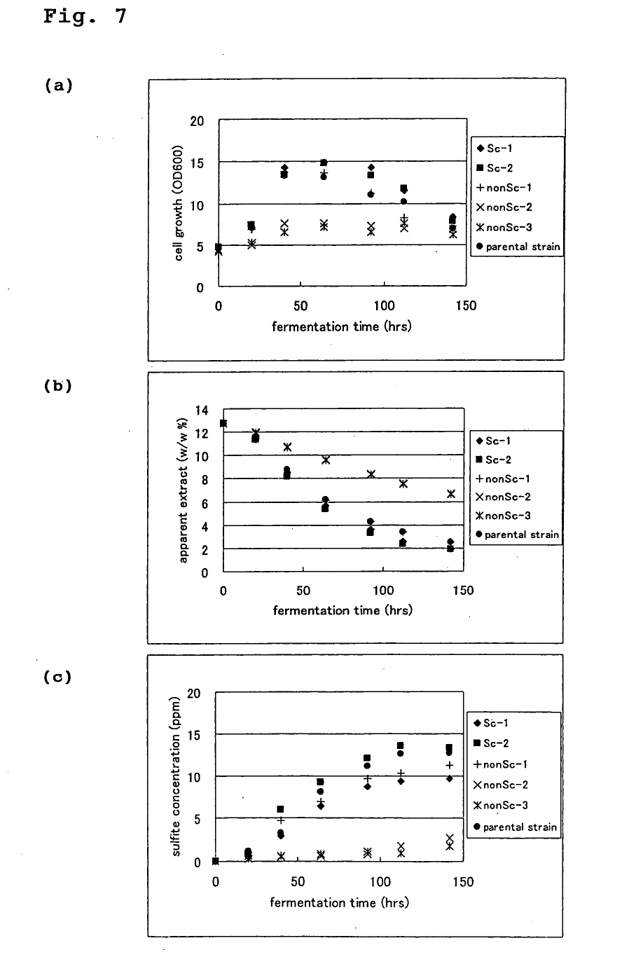

Wort is periodically sampled and monitored in cell growth (OD 600) (FIG. 7-(a)), apparent extract (FIG. 7-(b)) and sulfite concentration (FIG. 7-(c)). Quantitative analysis of sulfite in wort was carried out in such a method that it was captured in a hydrogen peroxide solution by means of distillation in an acidic condition and subjected to titration with an alkali (Revised Method for BCOJ Beer Analysis by the Brewing Society of Japan).

As a result, sulfite production in the wort by ScSSU1 disruptant was nearly same as that by parental strain, while it significantly decreased by non-ScSSU1 disruptant. It was suggested that non-ScSSU1 gene which is specific to bottom fermenting yeast greatly contributes to sulfite production in wort.

At the same time, growth rate and extract-consuming rate significantly were decreased in the non-ScSSU1 disruptant, and it supported that excessive sulfite in cells caused inhibition of cell growth.

### Example 12

### Overexpression of each SSU1 Gene

From the plasmid TOPO/non-ScSSU1 mentioned in Example 9, a fragment of about 1.5 kb including the full length of non-ScSSU1 ORF was excised by a treatment with a restriction enzyme (SacI-Bg1II). Then this fragment was inserted into a plasmid pNI-NUT which was similarly treated with a restriction enzyme (SacI-Bg1II) to construct a non-ScSSU1 overexpression vector pYI-non-ScSSU1 The vector pNI-NUT contains URA3 as a homologous recombination site and nourseothricin-resistance gene (nat1) and ampicillin-resistance gene (Amp^{r}) as selective markers. On the other hand, the ScSSU1 overexpression vector pNI-ScSSU1 has a structure wherein the non-ScSSU1 gene of the above-mentioned pYI-non-ScSSU1 has been substituted with the SSU1-R of about 2 kb derived from S. cerevisiae “J. Ferment. Bioeng., volume 86, page 427 (1998)”. For overexpression of each SSU1 gene, promoter and terminator of glyceraldehyde-3-phosphate dehydrogenase gene (TDH3) were used.

Bottom fermenting yeast BH225 was transformed by a overexpression vector prepared in the above-mentioned method. Transformation was carried out by a method mentioned in the Japanese Patent Laid-Open Gazette No. H07/303,475 and selected on YPD plate medium containing 50 mg/L of nourseothricin.

Confirmation of the overexpression was carried out by RT-PCR. Extraction of total RNA was carried out using an RNeasy Mini Kit (Qiagen), according manual of “for total RNA isolation from yeast” attached the kit. ScSSU1_for331 (SEQ ID NO: 23)/ScSSU1_{—}982rv (SEQ ID NO: 24) were used as ScSSU1-specific primers; non-ScSSU1_for329 (SEQ ID NO: 25)/non-ScSSU1_{—}981rv (SEQ ID NO: 26) were used as non-ScSSU1-specific primers; and PDA1_for1 (SEQ ID NO: 27)/PDA1_{—}730rv (SEQ ID NO: 28) were used as specific primers for constitutively expressed gene PDA1 used as an internal standard. PCR product was fractionated by 1.2% agarose electrophoresis, stained with an ethidium bromide solution and signal value of each SSU1 gene of transformant was normalized with a signal value of PDA 1 and compared with that of the parental strain. The overexpressed strains confirmed as such were named as ScSSU1 overexpressed strain and non-ScSSU1 overexpressed strain.

### Example 13

### Quantitative Analysis of Sulfite Production in a Fermentation Test

Fermentation test using-parental strain and each of SSU1 overexpressed strain obtained in the Example 12 was carried out under the following condition.

Original extract: 12.83%

Fermentation scale: 2 liters

Dissolved oxygen concentration: about 9 ppm

Fermentation temperature: 12° C.

Pitching rate: 10 g of wet yeast cells/2 L of wort

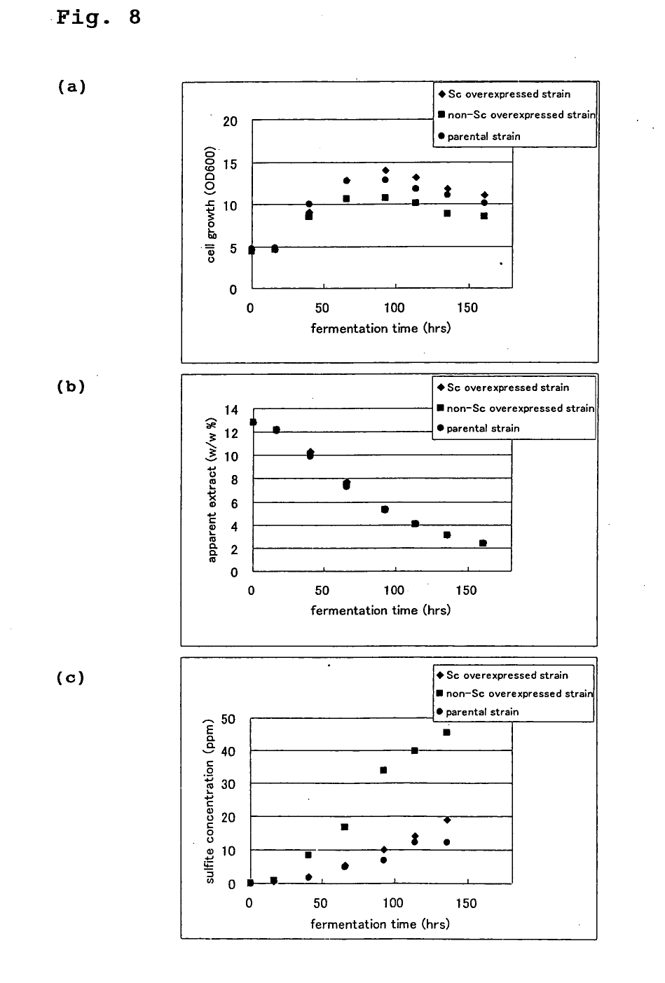

As same as in Example 11, wort is periodically sampled and monitored in cell growth (OD 600) (FIG. 8-(a)), apparent extract (FIG. 8-(b)) and sulfite concentration (FIG. 8-(c)). With regard to the sulfite production, it was only somewhat higher in Sc SSU1 overexpressed strain (19 ppm at the end of the fermentation) as compared with that of the parental strain (12 ppm at the same stage), while non-Sc SSU1 over expressed strain showed a significant increase (45 ppm at the same stage). At the same time, there was no difference in the growth rate and in the extract-consuming rate between the parental strain and the overexpressed strains.

From the above result, by overexpression of the gene encoding the sulfite-discharging pump specific to the bottom fermenting yeast shown in the present invention, it is possible to increase sulfite concentration in beer without changing the fermentation process and the fermentation period. As a result, it is now possible to produce an alcoholic beverage with excellent flavor stability and a longer quality preservation period.

### Example 14

### Cloning of MET14 Gene of Strain 34/70

DNA sequence of non-Sc MET14 gene was retrieved from the comparative database obtained in Example 6. A shotgun clone SSS 134_{—}021 containing about 1.9 kb (full-length) of non-Sc MET14 gene was obtained; its forward and reverse DNA sequence identity to S. cerevisiae were 79.0% and 56.0%, respectively.

The shotgun clone 134_{—}021 was selected from shotgun library and a full length of non-Sc MET14 gene was obtained by PCR. As a primer pair, synthetic DNAs of SacI-nonSc-MET14_for-21 (SEQ ID NO: 29) and BamHI-nonSc-MET14_rv618 (SEQ ID NO: 30) as shown in Table 3 were used. As a result of using such a pair of primers, a non-Sc MET14 gene (about 0.6 kb) embraced by SacI and BamHI restriction sites was obtained.

TABLE 3

| SEQ ID No. | Sequence Name | 5′-Base Sequence-3′ |
|---|---|---|
| 5 | M13_for | agtcacgacg ttgta |
| 6 | M13_rv | caggaaacag ctatgac |
| 7 | SS-cosF. 1 | aggcgtatca cgaggccctt tc |
| 8 | SS-cosR. 1 | cttatcgatg ataagcggtc aaacatgag |
| 9 | XVI-1 (L) cer-95894 | cgcaagctcc gtacgttcaa cattcttatg aacggc |
| 10 | XVI-1 (R) nonSc-106302rv | gcatcatcgt cgtgatcctt ctttggcaaa tgcagg |
| 11 | XVI-2 (L) cer-859737 | gcgggtattt tgatggtaaa tctacaagcc ctcggc |
| 12 | XVI-2 (R) nonSc-864595rv | cccagacaca gtttccagta tcatcctcgc agaac |
| 13 | SacI-nonScSSU1_for1 | gagctcatgg tcgctagttg gatgct |
| 14 | BglII-nonScSSU1_rv1460 | agatctcagc ttcagcccaa tccatt |
| 15 | SacI-ScSSU1_for1 | gagctcatgg ttgccaattg ggtact |
| 16 | BglII-ScSSU1_rv1406 | agatctctcc tacatgaaat gcttgc |
| 17 | nonScSSU1_for | atggtcgcta gttggatgct cactgccaca agggatttca |
| | | accctttcat atcgaatatt ctgtacagct gtttgtcatg |
| | | gttatggggg tcggtatttc ccttgacagt cttgacgtgc |
| 18 | nonScSSU1_rv | tgttaaatat gtactatcga tagccgagtt tgattcctcc |
| | | acactttcga acagtcttct ccgtcccttc ctctgataaa |
| | | tgctgttgaa aggagaattg cgcacttaac ttcgcatctg |
| 19 | ScSSU1_for | atggttgcca attgggtact tgctcttacg aggcagtttg |
| | | accccttcat gtttatgatg gtcatgggtg tcggcatttc |
| | | atcgaatatt ctatatagct ccttgacagt cttgacgtgc |
| 20 | ScSSU1_rv | ttatgctaaa cgcgtaaaat ctagagccga gtttgattct |
| | | tccacgcttt caatgctgtt atacggagaa actgtcgtct |
| | | tttccgtacc tgactctgaa cgcacttaac ttcgcatctg |
| 21 | nonScSSU1_for + pGAPAUR | atggtcgcta gttggatgct cactgccaca agggatttca |
| | | accctttcat gtttgtcatg gttatggggg tcggtatttc |
| | | atcgaatatt ctgtacagct ccggagctta ccagttctca |
| 22 | nonScSSU1_rv + AUR1-C | tgttaaatat gtactatcga tagccgagtt tgattcctcc |
| | | acactttcga tgctgttgaa aggagaattg acagtcttct |
| | | ccgtcccttc ctctgataaa tcgactctag aggatccaga |
| 23 | ScSSU1_for 331 | tcgaaagcga acacgacgaa |
| 24 | ScSSU1_982rv | cgacagaaat cacggtgaaa a |
| 25 | nonScSSU1_329 | tgtcacaaaa atttaccacg ac |
| 26 | nonScSSU1_981rv | aagggaaatt accgtaaaga ag |
| 27 | PDA1_for1 | atgtttgtcg cacctgtatc t |
| 28 | PDA1_730rv | gattagaggc accatcac |
| 29 | SacI-nonSc-MET14_for-21 | ctcgagctct cgtgaaattc attgaaacaa atg |
| 30 | BamHI-nonSc-MET14_rv618 | ggatccttat aagatttata gatgcttccg |
| 31 | SacI-ScMET14_for | ctcgagctca gaaaagttgg aattatttct cca |
| 32 | BamHI-ScMET14_rv | ggatccaatg tacagtaatc ggtcaaatta |

With regard to an Sc MET14 gene, a full length of the structural gene was obtained by PCR using a primer pair designed on the basis of the information of SGD and using genomic DNA of strain 34/70 as a template. Synthetic DNAs of SacI-ScMET14_for (SEQ ID NO: 31) and BamHI-ScMET14_rv (SEQ ID NO: 32) were used as primers. As a result of using such a pair of primers, Sc MET14 gene (about 0.6 kb) embraced by SacI and BamHI restriction sites was obtained.

The Sc MET14 and non-Sc MET14 genes obtained as above were inserted using a TA cloning kit (manufactured by Invitrogen) into pCR 2.1-TOPO vector attached to the kit, and they were named TOPO/ScMET14 and TOPO/nonSc-MET14, respectively.

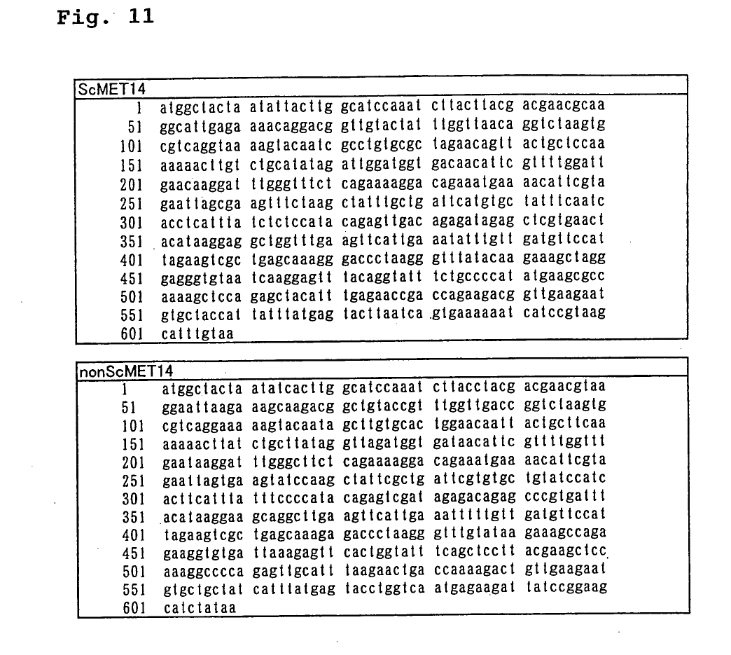

DNA sequences of the resulting Sc MET14 and non-Sc MET14 genes-were checked by a method by Sanger “Science, volume 214, page 1205 (1981)” (FIG. 11).

### Example 15

### Overexpression of each MET14 Gene in Sc SSU1 Overexpressed Strain

A fragment of about 0.6 kb containing Sc MET14 or non-Sc MET14 mentioned in Example 14 was inserted into a multi-cloning site of a expression vector pUP3GLP (Japanese Patent Laid-Open Gazette No. 2000/316,559) to construct overexpression vectors pUP3Sc MET14 and pUP3nonSc-MET14 in which each MET14 gene was expressed under control of glyceraldehyde-3-phosphate dehydrogenase promoter and terminator. On the other hand, top fermenting yeast, strain KN009F, was transformed by an Sc SSU1 overexpression vector pNI-SSU1 mentioned in Example 12 to prepare strain FOY227 which is an Sc SSU1 overexpressed strain. The strain FOY227 was transformed by the above pUP3ScMET14 and pUP3nonSc-MET14 to prepare strain FOY306 and strain FOY 307 in which Sc MET14 and non-Sc MET14, together with Sc SSU1, are overexpressed, respectively.

### Example 16

### Quantitative Analysis of Sulfite Production in a Fermentation Test

Fermentation tests were carried out under the following condition using strains prepared in Example 15; strain FOY227 which is an Sc SSU1 overexpressed strain, strain FOY306 which is an Sc MET14 overexpressed strain in strain FOY227, strain FOY307 which is a non-Sc MET14 overexpressed strain in strain FOY227and the parental strain KN009 F.

Original extract: 12.84%

Fermentation scale: 1.5 liters

Dissolved oxygen concentration: about 9 ppm

Fermentation temperature: 25° C. at all times

Pitching rate: 7.5 g of wet yeast cells/1.5 L of wort

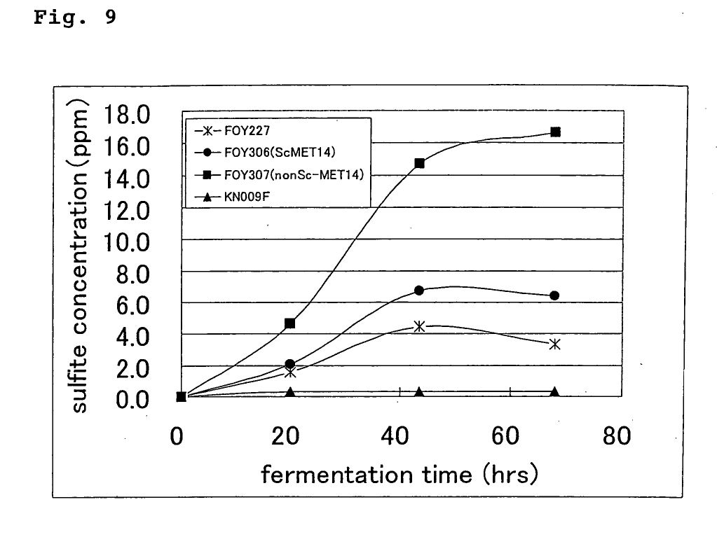

In the same way Example 11, wort is periodically sampled and monitored in cell growth (OD 600), apparent extract and sulfite concentration. With regard to the yeast growth and the consumed amount of extract, there was no difference among the strains. However, with regard to the sulfite production, it was only somewhat higher in Sc SSU1 overexpressed strain FOY227 (3.4 ppm at the end of the fermentation),and Sc MET14 and Sc SSU1 overexpressed strain FOY306 (6.4 ppm at the same stage) as compared with that of the parental strain KN009F(0.32 ppm at the same stage), while non-Sc MET14 and Sc SSU1 overexpressed strain FOY307 showed a significant increase (16.6 ppm at the same stage) as shown in FIG. 9.

From the above results, it was found that overexpression of the gene encoding the adenylyl sulfate kinase specific to the bottom fermenting yeast shown in the present invention was effective to increase sulfite concentration in beer without changing the fermentation process and the fermentation period. As a result, it is now possible to produce an alcoholic beverage with excellent flavor stability and a longer quality preservation period.

### Example 17

### Production of the Bottom Fermenting Yeast DNA Array

DNA array of bottom fermenting yeast was produced based on the DNA sequence information of the ORFs obtained in the above (h) and intergenic DNA sequences located between ORFs deduced from whole genome sequence of strain 34/70.

Production of the DNA Array

Based on the DNA sequence information from following four groups, PM probe (Perfect Match Probe; 25 base long) which is unique against whole genome sequence of the bottom fermenting yeast was designed using GeneChip® (Affymetrix) technology; (1) 15712 regions from the whole genome sequence information of strain 34/70, (2) 403 S. cerevisiae ORFs represented from SEQ ID NOS: 190811 to 191213 from SGD which are not identified as Sc type ORFs in strain 34/70, (3) 64 DNA sequences of genes represented from SEQ ID NOS: 195588 to 195651 used as internal standard.

In order to obtain quantitative and reproducible information, 8 to 11 probes for each region of (1) and (2), and 20 to 51 probes for each region of (3) were designed. To further increase the sensitivity and specificity of detection, mismatch probe (MM probe) that has sequences identical to PM probe with the exception of one mismatched base, which substitutes the complementary base (adenine (A) to thymine (T) and vice versa, or guanine (G) to cytosine (C) and vice versa) at the central position (i.e. base 13), was also designed.

All of designed PM and MM probes were synthesized and packed (manufactured by Affymetrix) to produce the DNA array using GeneChip® technology.

Group (1) Consists of:

A) 6204 DNA sequences of non-Sc type ORFs represented from SEQ ID NOS: 33 to 6236;

B) 7448 DNA sequences of Sc type ORFs represented from SEQ ID NOS: 75337 to 82784;

C) 28 DNA sequences of mitochondrial ORFs from 34/70 strain represented from SEQ ID NOS: 166154 to 166181;

D) 553 DNA sequences represented from SEQ ID NOS: 166490 to 167042, which have not been identified as the above ORFs but have significant similarity (E value <=10e-7) to the proteins of S. cerevisiae using NCBI-BlastX homology searching,

E) 1479 DNA sequences represented from SEQ ID NOS: 173125 to 174603, which have not been identified as the above A) or B) but have significant similarity (E-value <=10e⁻⁷) to the proteins of S. cerevisiae using NCBI-BlastP homology searching.

E-value is an expected value for a possibility that a query sequence is found in a data base by chance. For example, E-value 10 means that 10 sequences can be found accidentally. The smaller the E-value is, the lesser is the chance of finding the sequence by chance, and thus meaning that similarity between the found sequence in data base and the query sequence is significant.

The E-value as the result of homology searching such as NCBI-BLAST can be significant less than 10⁻⁷, preferably less than 10⁻¹⁰, more preferably less than 10⁻²⁰. The function can be assigned as having the least value with the DNA sequences of the ORFs as the result of the homology searching.

Group (2) Consists of:

YBL108C-A, YBR074W, YFL061W, YIL165C, YGR291C, YJR052W, YDR223W, YAL025C, YAR073W, YFL057C, YLL015W, YJR105W, YLR299C-A, YNR073C, YDL246C, YHL049C, YAR010C, YKL096W, YBL026W, YMR230W, YAL037C-A, YAL037C-B, YAL037W, YAL063C-A, YAL064C-A, YAL064W, YAL065C, YAL068W-A, YAL069W, YAR009C, YAR020C, YAR042W, YAR047C, YAR053W, YAR060C, YAR061W, YAR062W, YBL027W, YBL040C, YBLO68W-A, YBL101W-B, YBL109W, YBL112C, YBR092C, YBR191W-A, YBR219C, YCL019W, YCL029C, YCL065W, YCL066W, YCL068C, YCL069W, YCL073C, YCL074W, YCL075W, YCL076W, YCR035C, YCR036W, YCR038W-A, YCR101C, YCR104W, YCR105W, YCR106W, YCR107W, YCR108C, YDL003W, YDL037C, YDL064W, YDL073W, YDL094C, YDL095W, YDL096C, YDL136W, YDL143W, YDL152W, YDL191W, YDL200C, YDL201W, YDL247W-A, YDL248W, YDR014W, YDR015C, YDR034C-D, YDR039C, YDR045C, YDR098C-B, YDR160W, YDR210C-D, YDR210W-B, YDR215C, YDR225W, YDR261C-D, YDR261W-B, YDR292C, YDR302W, YDR304C, YDR305C, YDR342C, YDR344C, YDR364C, YDR365W-B, YDR427W, YDR433W, YDR471W, YDR510C-A, YDR543C, YDR544C, YEL012W, YEL075W-A, YER039C-A, YER046W-A, YER056C-A, YER060W-A, YER074W, YER138C, YER187W, YER188C-A, YER190C-A, YFL002W-A, YFL014W, YFL019C, YFL020C, YFL030W, YFL031W, YFL051C, YFL052W, YFL053W, YFL054C, YFL055W, YFL056C, YFL063W, YFL065C, YFL066C, YFL067W, YFR012W-A, YGL028C, YGL041C, YGL052W, YGL210W-A, YGL259W, YGL262W, YGL263W, YGR034W, YGR038C-B, YGR089W, YGR107W, YGR109W-A, YIL082W-A, YGR122C-A, YGR146C, YGR148C, YGR161W-B, YGR182C, YGR183C, YGR271C-A, YGR290W, YGR295C, YHL009W-A, YHL009W-B, YHL015W-A, YHL046W-A, YHL047C, YHL048C-A, YHL048W, YHR032C-A, YHR032W-A, YHR039C-A, YHR043C, YHR070C-A, YHR071C-A, YHR071W, YHR141C, YHR165W-A, YHR179W, YHR180C-B, YHR180W-A, YHR182W, YHR193C, YHR193C-A, YHR207C, YHR211W, YHR213W-A, YHR216W, YHR217C, YHR218W-A, YIL029C, YIL052C, YIL069C, YIL148W, YIL171W, YIL174W, YIL176C, YIR018C-A, YIR041W, YIR042C, YIR043C, YIR044C, YJL012C-A, YJL014W, YJL062W-A, YJL136C, YJL175W, YJL222W-B, YJR024C, YJR027W, YJR032W, YJR053W, YJR054W, YJR094W-A, YJR107W, YJR110W, YJR111C, YJR140W-A, YJR151C, YJR152W, YJR153W, YJR154W, YJR155W, YJR162C, YKL018W, YKL020C, YKL044W, YKL224C, YKL225W, YKR012C, YKR013W, YKR017C, YKR018C, YKR019C, YKR020W, YKR035C, YKR036C, YKR040C, YKR041W, YKR042W, YKR052C, YKR053C, YKR057W, YKR062W, YKR094C, YKR102W, YKR103W, YKR104W, YLL014W, YLL030C, YLL037W, YLL038C, YLL043W, YLL065W, YLR029C, YLR030W, YLR062C, YLR098C, YLR099W-A, YLR107W, YLR139C, YLR140W, YLR142W, YLR144C, YLR145W, YLR154C-G, YLR154W-A, YLR154W-B, YLR154W-C, YLR154W-E, YLR154W-F, YLR155C, YLR156W, YLR157C-B, YLR157W-C, YLR162W, YLR205C, YLR207W, YLR209C, YLR227W-B, .YLR236C, YLR237W, YLR238W, YLR245C, YLR251W, YLR271W, YLR278C, YLR287C-A, YLR305C, YLR306W, YLR311C, YLR317W, YLR338W, YLR344W, YLR345W, YLR354C, YLR364W, YLR380W, YLR401C, YLR402W, YLR410W-B, YLR411W, YLR412C-A, YLR412W, YLR413W, YLR448W, YLR460C, YLR461W, YLR463C, YLR465C, YML003W, YML039W, YML073C, YMR087W, YMR143W, YMR175W-A, YMR247W-A, YMR268W-A, YMR324C, YMR325W, YNL020C, YNL035C, YNL054W-B, YNL243W, YNR034W-A, YNR075C-A, YNR077C, YOL038C-A, YOL053W, YOL101C, YOL103W-B, YOL162W, YOL163W, YOL164W, YOL164W-A, YOL165C, YOL166C, YOL166W-A, YOR050C, YOR096W, YOR101W, YOR192C-B, YOR192C-C, YOR225W, YOR235W, YOR343W-B, YOR366W, YOR381W-A, YOR382W, YOR383C, YOR384W, YOR385W, YOR386W, YOR387C, YOR389W, YPL003W, YPL019C, YPL023C, YPL036W, YPL048W, YPL055C, YPL060C-A, YPL175W, YPL194W, YPL197C, YPL257W-B, YPR002C-A, YPR008W, YPR014C, YPR028W, YPR043W, YPR048W, YPR087W, YPR094W, YPR108W, YPR137C-B, YPR161C, YPR162C, YPR163C, YPR164W, YPR165W, YPR166C, YPR167C, YPR168W, YPR169W, YPR169W-A, YPR170C, YPR170W-A, YPR171W, YPR172W, YPR173C, YPR174C, YPR175W, YPR176C, YPR177C, YPR178W, YPR179C, YPR180W, YPR181C, YPR182W, YPR183W, YPR184W, YPR185W, YPR186C, YPR187W, YPR188C, YPR189W, and YPR190C Group (3) consists of (GenBank Accession No.):

AFFX-BioB-5_at (J04423.1), AFFX-BioB-M_at (J04423.1), AFFX-BioB-3_at (J04423.1), AFFX-BioC-5_at (J04423.1), AFFX-BioC-3_at (J04423.1), AFFX-BioDn-5_at (J04423.1), AFFX-BioDn-3_at (J04423.1), AFFX-CreX-5_at (X03453.1), AFFX-CreX-3_at (X03453.1), AFFX-DapX-5_at: (L38424.1), AFFX-DapX-M_at (L38424.1), AFFX-DapX-3_at (L38424.1), AFFX-LysX-5_at (X17013.1), AFFX-LysX-M_at (X17013.1), AFFX-LysX-3_at (X17013.1), AFFX-PheX-5_at (M24537.1), AFFX-PheX-M_at (M24537.1), AFFX-PheX-3_at (M24537.1), AFFX-ThrX-5_at (X04603.1), AFFX-ThrX-M_at (XQ4603.1), AFFX-ThrX-3_at (X04603.1), AFFX-TrpnX-5_at (K01391.1), AFFX-TrpnX-M_at (K01391.1), AFFX-TrpnX-3_at (K01391.1), AFFX-r2-Ec-bioB-5_at (J04423.1), AFFX-r2-Ec-bioB-M_at (J04423.1),AFFX-r2-Ec-bioB-3 at (J04423.1), AFFX-r2-Ec-bioC-5 at (J04423.1), AFFX-r2-Ec-bioC-3_at (J04423.1),AFFX-r2-Ec-bioD-5_at (J04423.1), AFFX-r2-Ec-bioD-3_at (J04423.1), AFFX-r2-P1-cre-5_at (X03453.1), AFFX-r2-P1-cre-3_at (X03453.1), AFFX-r2-Bs-dap-5_at (L38424.1), AFFX-r2-Bs-dap-M_at (L38424.1), AFFX-r2-Bs-dap-3_at (L38424.1), AFFX-r2-Bs-dap-3_at (X17013.1), AFFX-r2-Bs-lys-5_at (X17013.1), AFFX-r2-Bs-lys-M_at (X17013.1), AFFX-r2-Bs-phe-5_at (M24537.1), AFFX-r2-Bs-phe-M_at (M24537.1), AFFX-r2-Bs-phe-3_at (M24537.1), AFFX-r2-Bs-thr-5_s_at (X04603.1), AFFX-r2-Bs-thr-M_s_at (X04603.1), AFFX-r2-Bs- thr-3_s_at (X04603.1), AFFX-YFL039CS_at (V01288.1), AFFX-YFL039CM_at (V01288.1), AFFX-YFL039C3_at (V01288.1), AFFX-YER148w5_at (X16860.1), AFFX-YER148wM_at (X16860.1),AFFX-YER148w3_at (X16860.1), AFFX-YER022w5_at (L12026.1), AFFX-YER022wM_at (L12026.1), AFFX-YER022w3_at (L12026.1), AFFX-18srRnaa_at (Z75578.1), AFFX-18srRnab_at (Z75578.1), AFFX-18srRnac_at (Z75578.1), AFFX-18srRnad_at (Z75578.1), AFFX-18srRnae_at (Z75578.1), AFFX-25srRnaa_at (J01355.1), AFFX-25srRnab_at (J01355.1), AFFX-25srRnac_at (J01355.1), AFFX-25srRnad_at (J01355.1) and AFFX-25srRnae_at (J01355.1).

The probes from 6204 DNA sequences of non-Sc type ORFs described as above (1-A) were as follows:

The probes having the nucleotide sequence represented by SEQ ID NOS: 6237, 6238, 6239, 6240, 6241, 6242, 6243, 6244, 6245, 6246, 6247 were selected from the ORFs having the nucleotide sequence represented by SEQ ID NO: 33.

The probes having the nucleotide sequence represented by SEQ ID NOS: 6248, 6249, 6250, 6251, 6252, 6253, 6254, 6255, 6256, 6257, 6258 were selected from the ORFs having the nucleotide sequence represented by SEQ ID NO: 34.

The probes having the nucleotide sequence represented by SEQ ID NOS: 6259, 6260, 6261, 6262, 6263, 6264, 6265, 6266, 6267, 6268, 6269 were selected from the ORFs having the nucleotide sequence represented by SEQ ID NO: 35.

The probes having the nucleotide sequence represented by SEQ ID NOS: 6270, 6271, 6272, 6273, 6274, 6275, 6276, 6277, 6278, 6279, 6280 were selected from the ORFs having the nucleotide sequence represented by SEQ ID NO: 36.

The probes having the nucleotide sequence represented by SEQ ID NOS: 6281, 6282, 6283, 6284, 6285, 6286, 6287, 6288, 6289, 6290, 6291 were selected from the ORFs having the nucleotide sequence represented by SEQ ID NO: 37.

The probes having the nucleotide sequence represented by SEQ ID NOS: 6292, 6293, 6294, 6295, 6296, 6297, 6298, 6299, 6300, 6301, 6302 were selected from the ORFs having the nucleotide sequence represented by SEQ ID NO: 38.

The probes having the nucleotide sequence represented by SEQ ID NOS: 6303, 6304, 6305, 6306, 6307, 6308, 6309, 6310, 6311, 6312, 6313 were selected from the ORFs having the nucleotide sequence represented by SEQ ID NO: 39.

The probes having the nucleotide sequence represented by -SEQ ID NOS: 6314, 6315, 6316, 6317, 6318, 6319, 6320, 6321, 6322, 6323, 6324 were selected from the ORFs having the nucleotide sequence represented by SEQ ID NO: 40.

The probes having the nucleotide sequence represented by SEQ ID NOS: 6325, 6326, 6327, 6328, 6329, 6330, 6331, 6332, 6333, 6334, 6335 were selected from the ORFs having the nucleotide sequence represented by SEQ ID NO: 41.

The probes having the nucleotide sequence represented by SEQ ID NOS: 6336, 6337, 6338, 6339, 6340, 6341, 6342, 6343, 6344, 6345, 6346 were selected from the ORFs having the nucleotide sequence represented by SEQ ID NO: 42.

The probes having the nucleotide sequence represented by SEQ ID NOS: 6347, 6348, 6349, 6350, 6351, 6352, 6353, 6354, 6355, 6356, 6357 were selected from the ORFs having the nucleotide sequence represented by SEQ ID NO: 43.

The probes having the nucleotide sequence represented by SEQ ID NOS: 6358, 6359, 6360, 6361, 6362, 6363, 6364, 6365, 6366, 6367, 6368 were selected from the ORFs having the nucleotide sequence represented by SEQ ID NO: 44.

The probes having the nucleotide sequence represented by SEQ ID NOS: 6369, 6370, 6371, 6372, 6373, 6374, 6375, 6376, 6377, 6378, 6379 were selected from the ORFs having the nucleotide sequence represented by SEQ ID NO: 45.

The probes having the nucleotide sequence represented by SEQ ID NOS: 6380, 6381, 6382, 6383, 6384, 6385, 6386, 6387, 6388, 6389, .6390 were selected from the ORFs having the nucleotide sequence.represented by SEQ ID NO: 46.

The probes having the nucleotide sequence represented by SEQ ID NOS: 6391, 6392, 6393, 6394, 6395, 6396, 6397, 6398, 6399, 6400, 6401 were selected from the ORFs having the nucleotide sequence represented by SEQ ID NO: 47.

The probes having the nucleotide sequence represented by SEQ ID NOS: 6402, 6403, 6404, 6405, 6406, 6407, 6408, 6409, 6410, 6411, 6412 were selected from the ORFs having the nucleotide sequence represented by SEQ ID NO: 48.

The probes having the nucleotide sequence represented by SEQ ID NOS: 6413, 6414, 6415, 6416, 6417, 6418, 6419, 6420, 6421, 6422, 6423 were selected from the ORFs having the nucleotide sequence represented by SEQ ID NO: 49.

The probes having the nucleotide sequence represented by SEQ ID NOS: 6424, 6425, 6426, 6427, 6428, 6429, 6430, 6431, 6432, 6433, 6434 were selected from the ORFs having the nucleotide sequence represented by SEQ ID NO: 50.

The probes having the nucleotide sequence represented by SEQ ID NOS: 6435, 6436, 6437, 6438, 6439, 6440, 6441, 6442, 6443, .6444, 6445 were selected from the ORFs having the nucleotide sequence represented by SEQ ID NO: 51.

The probes having the nucleotide sequence represented by SEQ ID NOS: 6446, 6447, 6448, 6449; 6450, 6451, 6452, 6453, 6454, 6455, 6456 were selected from the ORFs having the nucleotide sequence represented by SEQ ID NO: 52.

The probes having the nucleotide sequence represented by SEQ ID NOS: 6457, 6458, 6459, 6460, 6461, 6462, 6463, 6464, 6465, 6466, 6467 were selected from the ORFs having the nucleotide sequence represented by SEQ ID NO: 53.

The probes having the nucleotide sequence represented by SEQ ID NOS: 6468, 6469, 6470, 6471, 6472, 6473, 6474, 6475, 6476, 6477, 6478 were selected from the ORFs having the nucleotide sequence represented by SEQ ID NO: 54.

The probes having the nucleotide sequence represented by SEQ ID NOS: 6479, 6480, 6481, 6482, 6483, 6484, 6485, 6486, 6487, 6488, 6489 were selected from the ORFs having the nucleotide sequence represented by SEQ ID NO: 55.

The probes having the nucleotide sequence represented by SEQ ID NOS: 6490, 6491, 6492, 6493, 6494, 6495, 6496, 6497, 6498, 6499, 6500 were selected from the ORFs having the nucleotide sequence represented by SEQ ID NO: 56.

The probes having the nucleotide sequence represented by SEQ ID NOS: 6501, 6502, 6503, 6504, 6505, 6506, 6507, 6508, 6509, 6510, 6511 were selected from the ORFs having the nucleotide sequence represented by SEQ ID NO: 57.

The probes having the nucleotide sequence represented by SEQ ID NOS: 6512, 6513, 6514, 6515, 6516, 6517, 6518, 6519, 6520, 6521, 6522 were selected from the OREs having the nucleotide sequence represented by SEQ ID NO: 58.

The probes having the nucleotide sequence represented by SEQ ID NOS: 6523, 6524, 6525, 6526, 6527, 6528, 6529, 6530, 6531, 6532, 6533 were selected from the ORFs having the nucleotide sequence represented by SEQ ID NO: 59.

The probes having the nucleotide sequence represented by SEQ ID NOS: 6534, 6535, 6536, 6537, 6538, 6539, 6540, 6541, 6542, 6543, 6544 were selected from the ORFs having the nucleotide sequence represented by SEQ ID NO: 60.

The probes having the nucleotide sequence represented by SEQ ID NOS: 6545, 6546, 6547, 6548, 6549, 6550, 6551, 6552, 6553, 6554, 6555 were selected from the ORFs having the nucleotide sequence represented by SEQ ID NO: 61.

The probes having the nucleotide sequence represented by SEQ ID NOS: 6556, 6557, 6558, 6559, 6560, 6561, 6562, 6563, 6564, 6565, 6566 were selected from the ORFs having the nucleotide sequence represented by SEQ ID NO: 62.

The probes having the nucleotide sequence represented by SEQ ID NOS: 6567, 6568, 6569, 6570, 6571, 6572, 6573, 6574, 6575, 6576, 6577 were selected from the ORFs having the nucleotide sequence represented by SEQ ID NO: 63.

The probes having the nucleotide sequence represented by SEQ ID NOS: 6578, 6579, 6580, 6581, 6582, 6583, 6584, 6585, 6586, 6587, 6588 were selected from the ORFs having the nucleotide sequence represented by SEQ ID NO: 64.

The probes having the nucleotide sequence represented by SEQ ID NOS: 6589, 6590, 6591, 6592, 6593, 6594, 6595, 6596, 6597, 6598, 6599 were selected from the ORFs having the nucleotide sequence represented by SEQ ID NO: 65.

The probes having the nucleotide sequence represented by SEQ ID NOS: 6600, 6601, 6602, 6603, 6604, 6605, 6606, 6607, 6608, 6609, 6610 were selected from the ORFs having the nucleotide sequence represented by SEQ ID NO: 66.

The probes having the nucleotide sequence represented by SEQ ID NOS: 6611, 6612, 6613, 6614, 6615, 6616, 6617, 6618, 6619, 6620, 6621 were selected from the ORFs having the nucleotide sequence represented by SEQ ID NO: 67.

The probes having the nucleotide sequence represented by SEQ ID NOS: 6622, 6623, 6624, 6625, 6626, 6627, 6628, 6629, 6630, 6631, 6632 were selected from the ORFs having the nucleotide sequence represented by SEQ ID NO: 68.

The probes having the nucleotide sequence represented by SEQ ID NOS: 6633, 6634, 6635, 6636, 6637, 6638, 6639, 6640, 6641, 6642, 6643 were selected from the ORFs having the nucleotide sequence represented by SEQ ID NO: 69.

The probes having the nucleotide sequence represented by SEQ ID NOS: 6644, 6645, 6646, 6647, 6648, 6649, 6650, 6651, 6652, 6653, 6654 were selected from the ORFs having the nucleotide sequence represented by SEQ ID NO: 70.

The probes having the nucleotide sequence represented by SEQ ID NOS: 6655, 6656, 6657, 6658, 6659, 6660, 6661, 6662, 6663, 6664, 6665 were selected from the ORFs having the nucleotide sequence represented by SEQ ID NO: 71.

The probes having the nucleotide sequence represented by SEQ ID NOS: 6666, 6667, 6668, 6669, 6670, 6671, 6672, 6673, 6674, 6675, 6676 were selected from the ORFs having the nucleotide sequence represented by SEQ ID NO: 72.

The probes having the nucleotide sequence represented by SEQ ID NOS: 6677, 6678, 6679, 6680, 6681, 6682, 6683, 6684, 6685, 6686, 6687 were selected from the ORFs having the nucleotide sequence represented by SEQ ID NO: 73.

The probes having the nucleotide sequence represented by SEQ ID NOS: 6688, 6689, 6690, 6691, 6692, 6693, 6694, 6695, 6696, 6697, 6698 were selected from the ORFs having the nucleotide sequence represented by SEQ ID NO: 74.

The probes having the nucleotide sequence represented by SEQ ID NOS: 6699, 6700, 6701, 6702, 6703, 6704, 6705, 6706, 6707, 6708, 6709 were selected from the ORFs having the nucleotide sequence represented by SEQ ID NO: 75.

The probes having the nucleotide sequence represented by SEQ ID NOS: 6710, 6711, 6712, 6713, 6714, 6715, 6716, 6717, 6718, 6719, 6720 were selected from the ORFs having the nucleotide sequence represented by SEQ ID NO: 76.

The probes having the nucleotide sequence represented by SEQ ID NOS: 6721, 6722, 6723, 6724, 6725, 6726, 6727, 6728, 6729, 6730, 6731 were selected from the ORFs having the nucleotide sequence represented by SEQ ID NO: 77.

The probes having the nucleotide sequence represented by SEQ ID NOS: 6732, 6733, 6734, 6735, 6736, 6737, 6738, 6739, 6740, 6741, 6742 were selected from the ORFs having the nucleotide sequence represented by SEQ ID NO: 78.

The probes having the nucleotide sequence represented by SEQ ID NOS: 6743, 6744, 6745, 6746, 6747, 6748, 6749, 6750, 6751, 6752, 6753 were selected from the ORFs having the nucleotide sequence represented by SEQ ID NO: 79.

The probes having the nucleotide sequence represented by SEQ ID NOS: 6754, 6755, 6756, 6757, 6758, 6759, 6760, 6761, 6762, 6763, 6764 were selected from the ORFs having the nucleotide sequence represented by SEQ ID NO: 80.

The probes having the nucleotide sequence represented by SEQ ID NOS: 6765, 6766, 6767, 6768, 6769, 6770, 6771, 6772, 6773, 6774, 6775 were selected from the ORFs having the nucleotide sequence represented by SEQ ID NO: 81.

The probes having the nucleotide sequence represented by SEQ ID NOS: 6776, 6777, 6778, 6779,.6780, 6781, 6782, 6783, 6784, 6785, 6786 were selected from the ORFs having the nucleotide sequence represented by SEQ ID NO: 82.

The probes having the nucleotide sequence represented by SEQ ID NOS: 6787, 6788, 6789, 6790, 6791, 6792, 6793, 6794, 6795, 6796, 6797 were selected from the ORFs having the nucleotide sequence represented by SEQ ID NO: 83.

The probes having the nucleotide sequence represented by SEQ ID NOS: 6798, 6799, 6800, 6801, 6802, 6803, 6804, 6805, 6806, 6807, 6808 were selected from the ORFs having the nucleotide sequence represented by SEQ ID NO: 84.

The probes having the nucleotide sequence represented by SEQ ID NOS: 6809, 6810, 6811, 6812, 6813, 6814, 6815, 6816, 6817, 6818, 6819 were selected from the ORFs having the nucleotide sequence represented by SEQ ID NO: 85.

The probes having the nucleotide sequence represented by SEQ ID NOS: 6820, 6821, 6822, 6823, 6824, 6825, 6826, 6827, 6828, 6829, 6830.were selected from the ORFs having the nucleotide sequence represented by SEQ ID NO: 86.

The probes having the nucleotide sequence represented by SEQ ID NOS: 6831, 6832, 6833, 6834, 6835, 6836, 6837, 6838, 6839, 6840, 6841 were selected from the ORFs having the nucleotide sequence represented by SEQ ID NO: 87.

The probes having the nucleotide sequence represented by SEQ ID NOS: 6842, 6843, 6844, 6845, 6846, 6847, 6848, 6849, 6850, 6851, 6852 were selected from the ORFs having the nucleotide sequence represented by SEQ ID NO: 88.

The probes having the nucleotide sequence represented by SEQ ID NOS.: 6853, 6854, 6855, 6856, 6857, 6858, 6859, 6860, 6861, 6862, 6863 were selected from the ORFs having the nucleotide sequence represented by SEQ ID NO: 89.

The probes having-the nucleotide sequence represented by SEQ ID NOS: 6864, 6865, 6866, 6867, 6868, 6869, 6870, 6871, 6872, 6873, 6874 were selected from the ORFs having the nucleotide sequence represented by SEQ ID NO: 90.

The probes having the nucleotide sequence represented by SEQ ID NOS: 6875, 6876, 6877, 6878, 6879, 6880, 6881, 6882, 6883, 6884, 6885 were selected from the ORFs having the nucleotide sequence represented by SEQ ID NO: 91.

The probes having the nucleotide sequence represented by SEQ ID NOS: 6886, 6887, 6888, 6889, 6890, 6891, 6892, 6893, 6894, 6895, 6896 were selected from the ORFs having the nucleotide sequence represented by SEQ ID NO: 92.

The probes having the nucleotide sequence represented by SEQ ID NOS: 6897, 6898, 6899, 6900, 6901, 6902, 6903, 6904, 6905, 6906, 6907 were selected from the ORFs having the nucleotide sequence represented by.SEQ ID NO: 93.

The probes having the nucleotide sequence represented by SEQ ID NOS: 6908, 6909, 6910, 6911, 6912, 6913, 6914, 6915, 6916, 6917, 6918 were selected from the ORFs having the nucleotide sequence represented by SEQ ID NO: 94.

The probes having the nucleotide sequence represented by SEQ ID NOS: 6919, 6920, 6921, 6922, 6923, 6924, 6925, 6926, 6927, 6928, 6929 were selected from the ORFs having the nucleotide sequence represented by SEQ ID NO: 95.

The probes having the nucleotide sequence represented by SEQ ID NOS: 693Q, 6931, 6932, 6933, 6934, 6935, 6936, 6937, 6938, 6939, 6940 were selected from the ORFs having the nucleotide sequence represented by SEQ ID NO: 96.

The probes having the nucleotide sequence represented by SEQ ID NOS: 6941, 6942, 6943, 6944, 6945, 6946, 6947, 6948, 6949, 6950, 69-51 were selected from the ORFs having the nucleotide sequence represented by SEQ ID NO: 97.

The probes having the nucleotide sequence represented by SEQ ID NOS: 6952, 6953, 6954, 6955, 6956, 6957, 6958, 6959, 6960, 6961, 6962 were selected from the ORFs having the nucleotide sequence represented by SEQ ID NO: 98.

The probes having the nucleotide sequence represented by SEQ ID NOS: 6963, 6964, 6965, 6966, 6967, 6968, 6969, 6970, 6971, 6972, 6973 were selected from the ORFs having the nucleotide sequence represented by SEQ ID NO: 99.

The probes having the nucleotide sequence represented by SEQ ID NOS: 6974, 6975, 6976, 6977, 6978, 6979, 6980, 6981, 6982, 6983, 6984 were selected from the ORFs having the nucleotide sequence represented by SEQ ID NO: 100.

The probes having the nucleotide sequence represented by SEQ ID NOS: 6985, 6986, 6987, 6988, 6989, 6990, 6991, 6992, 6993, 6994, 6995 were selected from the ORFs having the nucleotide sequence represented by SEQ ID NO: 101.

The probes having the nucleotide sequence represented by SEQ ID NOS: 6996, 6997, 6998, 6999, 7000, 7001, 7002, 7003, 7004, 7005, 7006 were selected from the ORFs having the nucleotide sequence represented by SEQ ID NO: 102.

The.probes having the nucleotide sequence represented by SEQ ID NOS: 7007, 7008, 7009, 7010, 7011, 7012, 7013, 7014, 7015, 7016, 7017 were selected from the ORFs having the nucleotide sequence represented by SEQ ID NO: 103.

The probes having the nucleotide sequence represented by SEQ ID NOS: 7018, 7019, 7020, 7021, 7022, 7023, 7024, 7025, 7026, 7027, 7028 were selected from the ORFs having the nucleotide sequence represented by SEQ ID NO: 104.

The probes having the nucleotide sequence represented by SEQ ID NOS: 7029, 7030, 7031, 7032, 7033, 7034, 7035, 7036, 7037, 7038, 7039 were selected from the ORFs having the nucleotide sequence represented by SEQ ID NO: 105.

The probes having the nucleotide sequence represented by SEQ ID NOS: 7040, 7041, 7042, 7043, 7044, 7045, 7046, 7047, 7048, 7049, 7050 were selected from the ORFs having the nucleotide sequence represented by SEQ ID NO: 106.

The probes having the nucleotide sequence represented by SEQ ID NOS: 7051, 7052, 7053, 7054, 7055, 7056, 7057, 7058, 7059, 7060, 7061 were selected from the ORFs having the nucleotide sequence represented by SEQ ID NO: 107.

The probes having the nucleotide sequence represented by SEQ ID NOS: 7062, 7063, 7064, 7065, 7066, 7067, 7068, 7069, 7070, 7071, 7072 were selected from the ORFs having the nucleotide sequence represented by SEQ ID NO: 108.

The probes having the nucleotide sequence represented by SEQ ID NOS: 7073, 7074, 7075, 7076, 7077, 7078, 7079, 7080, 7081, 7082, 7083 were selected from the ORFs having the nucleotide sequence represented by SEQ ID NO: 109.

The probes having the nucleotide sequence represented by SEQ ID NOS: 7084, 7085, 7086, 7087, 7088, 7089, 7090, 7091, 7092, 7093, 7094 were selected from the ORFs having the nucleotide sequence represented by SEQ ID NO: 110.

The probes having the nucleotide sequence represented by SEQ ID NOS: 7095, 7096, 7097, 7098, 7099, 7100, 7101, 7102, 7103, 7104, 7105 were selected from the ORFs having the nucleotide sequence represented by SEQ ID NO: 111.

The probes having the nucleotide sequence represented by SEQ ID NOS: 7106, 7107, 7108, 7109, 7110, 7111, 7112, 7113, 7114, 7115, 7116 were selected from the ORFs having the nucleotide sequence represented by SEQ ID NO: 112.

The probes having the nucleotide sequence represented by SEQ ID NOS: 7117, 7118, 7119, 7120, 7121, 7122, 7123, 7124, 7125 were selected from the ORFs having the nucleotide sequence represented by SEQ ID NO: 113.

The probes having the nucleotide sequence represented by SEQ ID NOS: 7126, 7127, 7128, 7129, 7130, 7131, 7132, 7133, 7134, 7135, 7136 were selected from the ORFs having the nucleotide sequence represented by SEQ ID NO: 114.

The probes having the nucleotide sequence represented by SEQ ID NOS: 7137, 7138, 7139, 7140, 7141, 7142, 7143, 7144, 7145, 7146, 7147 were selected from the ORFs having the nucleotide sequence represented by SEQ ID NO: 115.

The probes having the nucleotide sequence represented by SEQ ID NOS: 7148, 7149, 7150, 7151, 7152, 7153, 7154, 7155, 7156, 7157, 7158 were selected from the ORFs having the nucleotide sequence represented by SEQ ID NO: 116.

The probes having the nucleotide sequence represented by SEQ ID NOS: 7159, 7160, 7161, 7162, 7163, 7164, 7165, 7166, 7167, 7168, 7169 were selected from the ORFs having the nucleotide sequence represented by SEQ ID NO: 117.

The probes having the nucleotide sequence represented by SEQ ID NOS: 7170, 7171, 7172, 7173, 7174, 7175, 7176, 7177, 7178, 7179, 7180 were selected from the ORFs having the nucleotide sequence represented by SEQ ID NO: 118.

The probes having the nucleotide sequence represented by SEQ ID NOS: 7181, 7182,. 7183, 7184, 7185, 7186, 7187, 7188, 7189, 7190, 7191 were selected from the ORFs having the nucleotide sequence represented by SEQ ID NO: 119.

The probes having the nucleotide sequence represented by SEQ ID NOS: 7192, 7193, 7194, 7195, 7196, 7197, 7198, 7199, 7200, 7201, 7202 were selected from the ORFs having the nucleotide sequence represented by SEQ ID NO: 120.

The probes having the nucleotide sequence represented by SEQ ID NOS: 7203, 7204, 7205, 7206, 7207, 7208, 7209, 7210, 7211, 7212, 7213 were selected from the ORFs having the nucleotide sequence represented by SEQ ID NO: 121.

The probes having the nucleotide sequence represented by SEQ ID NOS: 7214, 7215, 7216, 7217, 7218, 7219, 7220, 7.221, 7222, 7223, 7224 were selected from the ORFs having the nucleotide sequence represented by SEQ ID NO: 122.

The probes having the nucleotide sequence represented by SEQ ID NOS: 7225, 7226, 7227, 7228, 7229, 7230, 7231, 7232, 7233, 7234, 7235 were selected from the ORFs having the nucleotide sequence represented by SEQ ID NO: 123.

The probes having the nucleotide sequence represented by SEQ ID NOS: 7236, 7237, 7238, 7239, 7240, 7241, 7242, 7243, 7244, 7245, 7246 were selected from the ORFs having the nucleotide sequence represented by SEQ ID NO: 124.

The probes having the nucleotide sequence represented by SEQ ID NOS: 7247, 7248, 7249, 7250, 7251, 7252, 7253, 7254, 7255, 7256, 7257 were selected from the ORFs having the nucleotide sequence represented by SEQ ID NO: 125.

The probes having the nucleotide sequence represented by SEQ ID NOS: 7258, 7259, 7260, 7261, 7262, 7263, 7264, 7265, 7266, 7267, 7268 were selected from the ORFs having the nucleotide sequence represented by SEQ ID NO: 126.

The probes having the nucleotide sequence represented by SEQ ID NOS: 7269, 7270, 7271, 7272, 7273, 7274, 7275, 7276, 7277, 7278, 7279 were selected from the ORFs having the nucleotide sequence represented by SEQ ID NO: 127.

The probes having the nucleotide sequence represented by SEQ ID NOS: 7280, 7281, 7282, 7283, 7284, 7285, 7286, 7287, 7288, 7289, 7290 were selected from the ORFs having the nucleotide sequence represented by SEQ ID NO: 128.

The probes having the nucleotide sequence represented by SEQ ID NOS: 7291, 7292, 7293, 7294, 7295, 7296, 7297, 7298, 7299, 7300, 7301 were selected from the ORFs having the nucleotide sequence represented by SEQ ID NO: 129.

The probes having the nucleotide sequence represented by SEQ ID NOS: 7302, 7303, 7304, 7305, 7306, 7307, 7308, 7309, 7310, 7311, 7312 were selected from the ORFs having the nucleotide sequence represented by SEQ ID NO: 130.

The probes having the nucleotide sequence represented by SEQ ID NOS: 7313, 7314, 7315, 7316, 7317, 7318, 7319, 7320, 7321, 7322, 7323 were selected from the ORFs having the nucleotide sequence represented by SEQ ID NO: 131.

The probes having the nucleotide sequence represented by SEQ ID NOS: 7324, 7325, 7326, 7327, 7328, 7329, 7330, 7331, 7332, 7333, 7334 were selected from the ORFs having the nucleotide sequence represented by SEQ ID NO: 132.

The probes having the nucleotide sequence represented by SEQ ID NOS: 7335, 7336, 7337, 7338, 7339, 7340, 7341, 7342, 7343, 7344, 7345 were selected from the ORFs having the nucleotide sequence represented by SEQ ID NO: 133.

The probes having the nucleotide sequence represented by SEQ ID NOS: 7346, 7347, 7348, 7349, 7350, 7351, 7352, 7353, 7354, 7355, 7356 were selected from the ORFs having the nucleotide sequence represented by SEQ ID NO: 134.

The probes having the nucleotide sequence represented by SEQ ID NOS: 7357, .7358, 7359, 7360, 7361, 7362, 7363, 7364, 7365, 7366, 7367 were selected from the ORFs having the nucleotide sequence represented by SEQ ID NO: 135.

The probes having the nucleotide sequence represented by SEQ ID NOS: 7368, 7369, 7370, 7371, 7372, 7373, 7374, 7375, 7376, 7377, 7378 were selected from the ORFs having the nucleotide sequence represented by SEQ ID NO: 136.

The probes having the nucleotide sequence represented by SEQ ID NOS: 7379, 7380, 7381, 7382, 7383, 7384, 7385, 7386, 7387, 7388, 7389 were selected from the ORFs having the nucleotide sequence represented by SEQ ID NO: 137.

The probes having the nucleotide sequence represented by SEQ ID NOS: 7390, 7391, 7392, 7393, 7394, 7395, 7396, 7397, 7398, 7399, 7400 were selected from the ORFs having the nucleotide sequence represented by SEQ ID NO: 138.

The probes having the nucleotide sequence represented by SEQ ID NOS: 7401, 7402, 7403, 7404, 7405, 7406, 7407, 7408, 7409, 7410, 7411 were selected from the ORFs having the nucleotide sequence represented by SEQ ID NO: 139.

The probes having the nucleotide sequence represented by SEQ ID NOS: 7412, 7413, 7414, 7415, 7416, 7417, 7418, 7419, 7420, 7421, 7422 were selected from the ORFs having the nucleotide sequence represented by SEQ ID NO: 140.

The probes having the nucleotide sequence represented by SEQ ID NOS: 7423, 7424, 7425, 7426, 7427, 7428, 7429, 7430, 7431, 7432, 7433 were selected from the ORFs having the nucleotide sequence represented by SEQ ID NO: 141.

The probes having the nucleotide sequence represented by SEQ ID NOS: 7434, 7435, 7436, 7437, 7438, 7439, 7440, 7441, 7442, 7443, 7444 were selected from the ORFs having the nucleotide sequence represented by SEQ ID NO: 142.

The probes having the nucleotide sequence represented by SEQ ID NOS: 7445, 7446, 7447, 7448, 7449, 7450, 7451, 7452, 7453, 7454, 7455 were selected from the ORFs having the nucleotide sequence represented by SEQ ID NO: 143.

The probes having the nucleotide-sequence represented by SEQ ID NOS: 7456, 7457, 7458, 7459, 7460, 7461, 7462, 7463, 7464, 7465, 7466 were selected from the ORFs having the nucleotide sequence represented by SEQ ID NO: 144.

The probes having the nucleotide sequence represented by SEQ ID NOS: 7467, 7468, 7469, 7470, 7471, 7472, 7473, 7474, 7475, 7476, 7477 were selected from the ORFs having the nucleotide sequence represented by SEQ ID NO: 145.

The probes having the nucleotide sequence represented by SEQ ID NOS: 7478, 7479, 7480, 7481, 7482, 7483, 7484, 7485, 7486, 7487, 7488 were selected from the ORFs having the nucleotide sequence represented by SEQ ID NO: 146.

The probes having the nucleotide sequence represented by SEQ ID NOS: 7489, 7490, 7491, 7492, 7493, 7494, 7495, 7496, 7497, 7498, 7499 were selected from the ORFs having the nucleotide sequence represented by SEQ ID NO: 147.

The probes having the nucleotide sequence represented by SEQ ID NOS: 7500, 7501, 7502, 7503, 7504, 7505, 7506, 7507, 7508, 7509, 7510 were selected from the ORFs having the nucleotide sequence represented by SEQ ID NO: 148.

The probes having the nucleotide sequence represented by SEQ ID NOS: 7511, 7512, 7513, 7514, 7515, 7516, 7517, 7518, 7519, 7520, 7521 were selected from the ORFs having the nucleotide sequence represented by SEQ ID NO: 149.

The probes having the nucleotide sequence represented by SEQ ID NOS: 7522, 7523, 7524, 7525, 7526, 7527, 7528, 7529, 7530, 7531, 7532 were selected from the ORFs having the nucleotide sequence represented by SEQ ID NO: 150.

The probes having the nucleotide sequence represented by SEQ ID NOS: 7533, 7534, 7535, 7536, 7537, 7538, 7539, 7540, 7541, 7542, 7543 were selected from the ORFs having the nucleotide sequence represented by SEQ ID NO: 151.

The probes having the nucleotide sequence represented by SEQ ID NOS: 7544, 7545, 7546, 7547, 7548, 7549, 7550, 7551, 7552, 7553, 7554 were selected from the ORFs having the nucleotide sequence represented by SEQ ID NO: 152.

The probes having the nucleotide sequence represented by SEQ ID NOS: 7555, 7556, 7557, 7558, 7559, 7560, 7561, 7562, 7563, 7564, 7565 were selected from the ORFs having the nucleotide sequence represented by SEQ ID NO: 153.

The probes having the nucleotide sequence represented by SEQ ID NOS: 7566, 7567, 7568, 7569, 7570, 7571, 7572, 7573, 7574, 7575, 7576 were selected from the ORFs having the nucleotide sequence represented by SEQ ID NO: 154.

The probes having the nucleotide sequence represented by SEQ ID NOS: 7577, 7578, 7579, 7580, 7581, 7582, 7583, 7584, 7585, 7586, 7587 were selected from the ORFs having the nucleotide sequence represented by SEQ ID NO: 155.

The probes having the nucleotide sequence represented by SEQ ID NOS: 7588, 7589, 7590, 7591, 7592, 7593, 7594, 7595, 7596, 7597, 7598 were selected from the ORFs having the nucleotide sequence represented by SEQ ID NO: 156.

The probes having the nucleotide sequence represented by SEQ ID NOS: 7599, 7600, 7601, 7602, 7603, 7604, 7605, 7606, 7607, 7608, 7609 were selected from the ORFs having the nucleotide sequence represented by SEQ ID NO: 157.

The probes having the nucleotide sequence represented by SEQ ID NOS: 7610, 7611, 7612, 7613, 7614, 7615, 7616, 7617, 7618, 7619, 7620 were selected from the ORFs having the nucleotide sequence represented by SEQ ID NO: 158.

The probes having the nucleotide sequence represented by SEQ ID NOS: 7621, 7622, 7623, 7624, 7625, 7626, 7627, 7628, 7629, 7630, 7631 were selected from the ORFs having the nucleotide sequence represented by SEQ ID NO: 159.

The probes having the nucleotide sequence represented by SEQ ID NOS: 7632, 7633, 7634, 7635, 7636, 7637, 7638, 7639, 7640, 7641, 7642 were selected from the ORFs having the nucleotide sequence represented by SEQ ID NO: 160.

The probes having the nucleotide sequence represented by SEQ ID NOS: 7643, 7644, 7645, 7646, 7647, 7648, 7649, 7650, 7651, 7652, 7653 were selected from the ORFs having the nucleotide sequence represented by SEQ ID NO: 161.

The probes having the nucleotide sequence represented by SEQ ID NOS: 7654, 7655, 7656, 7657, 7658, 7659, 7660, 7661, 7662, 7663, 7664 were selected from the ORFs having the nucleotide sequence represented by SEQ ID NO: 162.

The probes having the nucleotide sequence represented by SEQ ID NOS: 7665, 7666, 7667, 7668, 7669, 7670, 7671, 7672, 7673, 7674, 7675 were selected from the ORFs having the nucleotide sequence represented by SEQ ID NO: 163.

The probes having the nucleotide sequence represented by SEQ ID NOS: 7676, 7677, 7678, 7679, 7680, 7681, 7682, 7683, 7684, 7685, 7686 were selected from the ORFs having the nucleotide sequence represented by SEQ ID NO: 164.

The probes having the nucleotide sequence represented by SEQ ID NOS: 7687, 7688, 7689, 7690, 7691, 7692, 7693, 7694, 7695, 7696, 7697 were selected from the ORFs having the nucleotide sequence represented by SEQ ID NO: 165.

The probes having the nucleotide sequence represented by SEQ ID NOS: 7698, 7699, 7700, 7701, 7702, 7703, 7704, 7705, 7706, 7707, 7708 were selected from the ORFs having the nucleotide sequence represented by SEQ ID NO: 166.

The probes having the nucleotide sequence represented by SEQ ID NOS: 7709, 7710, 7711, 7712, 7713, 7714, 7715, 7716, 7717, 7718, 7719 were selected from the ORFs having the nucleotide sequence represented by SEQ ID NO: 167.

The probes having the nucleotide sequence represented by SEQ ID NOS: 7720, 7721, 7722, 7723, 7724, 7725, 7726, 7727, 7728, 7729, 7730 were selected from the ORFs having the nucleotide sequence represented by SEQ ID NO: 168.

The probes having the nucleotide sequence represented by SEQ ID NOS: 7731, 7732, 7733, 7734, 7735, 7736, 7737, 7738, 7739, 7740, 7741 were selected from the ORFs having the nucleotide sequence represented by SEQ ID NO: 169.

The probes having the nucleotide sequence represented by SEQ ID NOS: 7742, 7743, 7744, 7745, 7746, 7747, 7748, 7749, 7750, 7751, 7752 were selected from the ORFs having the nucleotide sequence represented by SEQ ID NO: 170.

The probes having the nucleotide sequence represented by SEQ ID NOS: 7753, 7754, 7755, 7756, 7757, 7758, 7759, 7760, 7761, 7762, 7763 were selected from the ORFs having the nucleotide sequence represented by SEQ ID NO: 171.

The probes having the nucleotide sequence represented by SEQ ID NOS: 7764, 7765, 7766, 7767, 7768, 7769, 7770, 7771, 7772, 7773, 7774 were selected from the ORFs having the nuclebtide sequence represented by SEQ ID NO: 172.

The probes having the nucleotide sequence represented by SEQ ID NOS: 7775, 7776, 7777, 7778, 7779, 7780, 7781, 7782, 7783, 7784, 7785 were selected from the ORFs having the nucleotide sequence represented by SEQ ID NO: 173.

The probes having the nucleotide sequence represented by SEQ ID NOS:. 7786, 7787, 7788, 7789, 7790, 7791, 7792, 7793, 7794, 7795, 7796 were selected from the ORFs having the nucleotide sequence represented by SEQ ID NO: 174.

The probes having the nucleotide sequence represented by SEQ ID NOS: 7797, 7798, 7799, 7800, 7801, 7802, 7803, 7804, 7805, 7806, 7807 were selected from the ORFs having the nucleotide sequence represented by SEQ ID NO: 175.

The probes having the nucleotide sequence represented by SEQ ID NOS: 7808, 7809, 7810, 7811, 7812, 7813, 7814, 7815, 7816, 7817, 7818 were selected from the ORFs having the nucleotide sequence represented by SEQ ID NO: 176.

The probes having the nucleotide sequence represented by SEQ ID NOS: 7819, 7820, 7821, 7822, 7823, 7824, 7825, 7826, 7827, 7828, 7829 were selected from the ORFs having the nucleotide sequence represented by SEQ ID NO: 177.

The probes having the nucleotide sequence represented by SEQ ID NOS: 7830, 7831, 7832, 7833, 7834, 7835, 7836, 7837, 7838, 7839, 7840 were selected from the ORFs having the nucleotide sequence represented by SEQ ID NO: 178.

The probes having the nucleotide sequence represented by SEQ ID NOS: 7841, 7842, 7843, 7844, 7845, 7846, 7847, 7848, 7849, 7850, 7851 were selected from the ORFs having the nucleotide sequence represented by SEQ ID NO: 179.

The probes having the nucleotide sequence represented by SEQ ID NOS: 7852, 7853, 7854, 7855, 7856, 7857, 7858, 7859, 7860, 7861, 7862 were selected from the ORFs having the nucleotide sequence represented by SEQ ID NO: 180.

The probes having the nucleotide sequence represented by SEQ ID NOS: 7863, 7864, 7865, 7866, 7867, 7868, 7869, 7870, 7871, 7872, 7873 were selected from the ORFs having the nucleotide sequence represented by SEQ ID NO: 181.

The probes having the nucleotide sequence represented by SEQ ID NOS: 7874, 7875, 7876, 7877, 7878, 7879, 7880, 7881, 7882, 7883, 7884 were selected from the ORFs having the nucleotide sequence represented by SEQ ID NO: 182.

The probes having the nucleotide sequence represented by SEQ ID NOS: 7885, 7886, 7887, 7888, 7889, 7890, 7891, 7892, 7893, 7894, 7895 were selected from the ORFs having the nucleotide sequence-represented by SEQ ID NO: 183.

The probes having the nucleotide sequence represented by SEQ ID NOS: 7896, 7897, 7898, 7899, 7900, 7901, 7902, 7903, 7904, 7905, 7906 were selected from the ORFs having the nucleotide sequence represented by SEQ ID NO: 184.

The probes having the nucleotide sequence represented by SEQ ID NOS: 7907, 7908, 7909, 7910, 7911, 7912, 7913, 7914, 7915, 7916, 7917 were selected from the ORFs having the nucleotide sequence represented by SEQ ID NO: 185.

The probes having the nucleotide sequence represented by SEQ ID NOS: 7918, 7919, 7920, 7921, 7922, 7923, 7924, 7925, 7926, 7927, 7928 were selected from the ORFs having the nucleotide sequence represented by SEQ ID NO: 186.

The probes having the nucleotide sequence represented by SEQ ID NOS: 7929, 7930, 7931, 7932, 7933, 7934, 7935, 7936, 7937, 7938, 7939 were selected from the ORFs having the nucleotide sequence represented by SEQ ID NO: 187.

The probes having the nucleotide sequence represented by SEQ ID NOS: 7940, 7941, 7942, 7943, 7944, 7945, 7946, 7947, 7948, 7949, 7950 were selected from the ORFs having the nucleotide sequence represented by SEQ ID NO: 188.

The probes having the nucleotide sequence represented by SEQ ID NOS: 7951, 7952, 7953, 7954, 7955, 7956, 7957, 7958, 7959, 7960, 7961 were selected from the ORFs having the nucleotide sequence represented by SEQ ID NO: 189.

The probes having the nucleotide sequence represented by SEQ ID NOS: 7962, 7963, 7964, 7965, 7966, 7967, 7968, 7969, 7970, 7971, 7972 were selected from the ORFs having the nucleotide sequence represented by SEQ ID NO: 190.

The probes having the nucleotide sequence represented by SEQ ID NOS: 7973, 7974, 7975, 7976, 7977, 7978, 7979, 7980, 7981, 7982, 7983 were selected from the ORFs having the nucleotide sequence represented by SEQ ID NO: 191.

The probes having the nucleotide sequence represented by SEQ ID NOS: 7984, 7985, 7986, 7987, 7988, 7989, 7990, 7991, 7992, 7993, 7994 were selected from the ORFs having the nucleotide sequence represented by SEQ ID NO: 192.

The probes having the nucleotide sequence represented by SEQ ID NOS: 7995, 7996, 7997, 7998, 7999, 8000, 8001, 8002, 8003, 8004, 8005 were selected from the,ORFs having the nucleotide sequence represented by SEQ ID NO: 193.

The probes having the nucleotide sequence represented by SEQ ID NOS: 8006, 8007, 8008, 8009, 8010, 8011, 8012, 8013, 8014, 8015, 8016 were selected from the ORFs having the nucleotide sequence represented by SEQ ID NO: 194.

The probes having the nucleotide sequence represented by SEQ ID NOS: 8017, 8018, 8019, 8020, 8021., 8022, 8023, 8024, 8025, 8026, 8027 were selected from the ORFs having the nucleotide sequence represented by SEQ ID NO: 195.

The probes having the nucleotide sequence represented by SEQ ID NOS: 8028, 8029, 8030, 8031, 8032, 8033, 8034, 8035, 8036, 8037, 8038 were selected from the ORFs having the nucleotide sequence represented by SEQ ID NO: 196.

The probes having the nucleotide sequence represented by SEQ ID NOS: 8039, 8040, 8041, 8042, 8043, 8044, 8045, 8046, 8047, 8048, 8049 were selected from the ORFs having the nucleotide sequence represented by SEQ ID NO: 197.

The probes having the nucleotide sequence represented by SEQ ID NOS: 8050, 8051, 8052, 8053, 8054, 8055, 8056, 8057, 8058, 8059, 8060 were selected from the ORFs having the nucleotide sequence represented by SEQ ID NO: 198.

The probes having the nucleotide sequence represented by SEQ ID NOS: 8061, 8062, 8063, 8064, 8065, 8066, 8067, 8068, 8069, 8070, 8071 were selected from the ORFs having the nucleotide sequence represented by SEQ ID NO: 199.

The probes having the nucleotide sequence represented by SEQ ID NOS: 8072, 8073, 8074, 8075, 8076, 8077, 8078, 8079, 8080, 8081, 8082 were selected from the ORFs having the nucleotide sequence represented by SEQ ID NO: 200.

The probes having the nucleotide sequence represented by SEQ ID NOS: 8083, 8084, 8085, 8086, 8087, 8088, 8089, 8090, 8091, 8092, 8093 were selected from the ORFs having the nucleotide sequence represented by SEQ ID NO: 201.

The probes having the nucleotide sequence represented by SEQ ID NOS: 8094, 8095, 8096, 8097, 8098, 8099, 8100, 8101, 8102, 8103, 8104 were selected from the ORFs having the nucleotide sequence represented by SEQ ID NO: 202.

The probes having the nucleotide sequence represented by SEQ ID NOS: 8105, 8106, 8107, 8108, 8109, 8110, 8111, 8112, 8113, 8114, 8115 were selected from the ORFs having the nucleotide sequence represented by SEQ ID NO: 203.

The probes having the nucleotide sequence represented by SEQ ID NOS: 8116, 8117, 8118, 8119, 8120, 8121, 8122, 8123, 8124, 8125, 8126 were selected from the ORFs having the nucleotide sequence represented by SEQ ID NO: 204.

The probes having the nucleotide sequence represented by SEQ ID NOS: 8127, 8128, 8129, 8130, 8131, 8132, 8133, 8134, 8135, 8136, 8137 were selected from the ORFs having the nucleotide sequence represented by SEQ ID NO: 205.

The probes having the nucleotide sequence represented by SEQ ID NOS: 8138, 8139, 8140, 8141, 8142, 8143, 8144, 8145, 8146, 8147, 8148 were selected from the ORFs having the nucleotide sequence represented by SEQ ID NO: 206.

The probes having the nucleotide sequence represented by SEQ ID NOS: 8149, 8150, 8151, 8152, 8153, 8154, 8155, 8156, 8157, 8158, 8159 were selected from the ORFs having the nucleotide sequence represented by SEQ ID NO: 207.

The probes having the nucleotide sequence represented by SEQ ID NOS: 8160, 8161, 8162, 8163, 8164, 8165, 8166, 8167, 8168, 8169, 8170 were selected from the ORFs having the nucleotide sequence represented by SEQ ID NO: 208.

The probes having the nucleotide sequence represented by SEQ ID NOS: 8171, 8172, 8173, 8174, 8175, 8176, 8177, 8178, 8179, 8180, 8181 were selected from the ORFs having the nucleotide sequence represented by SEQ ID NO: 209.

The probes having the nucleotide sequence represented by SEQ ID NOS: 8182, 8183, 8184, 8185, 8186, 8187, 8188, 8189, 8190, 8191, 8192 were selected from the ORFs having the nucleotide sequence represented by SEQ ID NO: 210.

The probes having the nucleotide sequence represented by SEQ ID NOS: 8193, 8194, 8195, 8196, 8197, 8198, 8199, 8200, 8201, 8202, 8203 were selected from the ORFs having the nucleotide sequence represented by SEQ ID NO: 211.

The probes having the nucleotide sequence represented by SEQ ID NOS: 8204, 8205, 8206, 8207, 8208, 8209, 8210, 8211, 8212, 8213, 8214 were selected from the ORFs having the nucleotide sequence represented by SEQ ID NO: 212.

The probes having the nucleotide sequence represented by SEQ ID NOS: 8215, 8216, 8217, 8218, 8219, 8220, 8221, 8222, 8223, 8224, 8225 were selected from the ORFs having the nucleotide sequence represented by SEQ ID NO: 213.

The probes having the nucleotide sequence represented by SEQ ID NOS: 8226, 8227, 8228, 8229, 8230, 8231, 8232, 8233, 8234, 8235, 8236 were selected from the ORFs having the nucleotide sequence represented by SEQ ID NO: 214.

The probes having the nucleotide sequence represented by SEQ ID NOS: 8237, 8238, 8239, 8240, 8241, 8242, 8243, 8244, 8245, 8246, 8247 were selected from the ORFs having the nucleotide sequence represented by SEQ ID NO: 215.

The probes having the nucleotide sequence represented by SEQ ID NOS: 8248, 8249, 8250, 8251, 8252, 8253, 8254, 8255, 8256, 8257, 8258 were selected from the ORFs having the nucleotide sequence represented by SEQ ID NO: 216.

The probes having the nucleotide sequence represented by SEQ ID NOS: 8259, 8260, 8261, 8262, 8263, 8264, 8265, 8266, 8267, 8268, 8269 were selected from the ORFs having the nucleotide sequence represented by SEQ ID NO: 217.

The probes having the nucleotide sequence represented by SEQ ID NOS: 8270, 8271, 8272, 8273, 8274, 8275, 8276, 8277, 8278, 8279, 8280 were selected from the ORFs having the nucleotide sequence represented by SEQ ID NO: 218.

The probes having the nucleotide sequence represented by SEQ ID NOS: 8281, 8282, 8283, 8284, 8285, 8286, 8287, 8288, 8289, 8290, 8291 were selected from the ORFs having the nucleotide sequence represented by SEQ ID NO: 219.

The probes having the nucleotide sequence represented by SEQ ID NOS: 8292, 8293, 8294, 8295, 8296, 8297, 8298, 8299, 8300, 8301, 8302 were selected from the ORFs having the nucleotide sequence represented by SEQ ID NO: 220.

The probes having the nucleotide sequence represented by SEQ ID NOS: 8303, 8304, 8305, 8306, 8307, 8308, 8309, 8310, 8311, 8312, 8313 were selected from the ORFs having the nucleotide sequence represented by SEQ ID NO: 221.

The probes having the nucleotide sequence represented by SEQ ID NOS: 8314, 8315, 8316, 8317, 8318, 8319, 8320, 8321, 8322, 8323, 8324 were selected from the ORFs having the nucleotide sequence represented by SEQ ID NO: 222.

The probes having the nucleotide sequence represented by SEQ ID NOS: 8325, 8326, 8327, 8328, 8329, 8330, 8331, 8332, 8333, 8334, 8335 were selected from the ORFs having the nucleotide sequence represented by SEQ ID NO: 223.

The probes having the nucleotide sequence represented by SEQ ID NOS: 8336, 8337, 8338, 8339, 8340, 8341, 8342, 8343, 8344, 8345, 8346 were selected from the ORFs having the nucleotide sequence represented by SEQ ID NO: 224.

The probes having the nucleotide sequence represented by SEQ ID NOS: 8347, 8348, 8349, 8350, 8351, 8352, 8353, 8354, 8355, 8356, 8357 were selected from the ORFs having the nucleotide sequence represented by SEQ ID NO: 225.

The probes having the nucleotide sequence represented by. SEQ ID NOS: 8358, 8359, 8360, 8361, 8362, 8363, 8364, 8365, 8366, 8367, 8368 were selected from the ORFs having the nucleotide sequence represented by SEQ ID NO: 226.

The probes having the nucleotide sequence represented by SEQ ID NOS: 8369, 8370, 8371, 8372, 8373, 8374, 8375, 8376, 8377, 8378, 8379 were selected from the ORFs having the nucleotide sequence represented by SEQ ID NO: 227.

The probes having the nucleotide sequence represented by SEQ ID NOS: 8380, 8381, 8382, 8383, 8384, 8385, 8386, 8387, 8388, 8389, 8390 were selected from the ORFs having the nucleotide sequence represented by SEQ ID NO: 228.

The probes having the nuclebtide sequence-represented by SEQ ID NOS: 8391, 8392, 8393, 8394, 8395, 8396, 8397, 8398, 8399, 8400, 8401 were selected from the ORFs having the nucleotide sequence represented by SEQ ID NO: 229.

The probes having the nucleotide sequence represented by SEQ ID NOS: 8402, 8403, 8404, 8405, 8406, 8407, 8408, 8409, 8410, 8411, 8412 were selected from the ORFs having the nucleotide sequence represented by SEQ ID NO: 230.

The probes having the nucleotide sequence represented by SEQ ID NOS: 8413, 8414, 8415, 8416, 8417, 8418, 8419, 8420, 8421, 8422, 8423 were selected from the ORFs having the nucleotide sequence represented by SEQ ID NO: 231.

The probes having the nucleotide sequence represented by SEQ ID NOS: 8424, 8425, 8426, 8427, 8428, 8429, 8430, 8431, 8432, 8433, 8434 were selected from the ORFs having the nucleotide sequence represented by SEQ ID NO: 232.

The probes having the nucleotide sequence represented by SEQ ID NOS: 8435, 8436, 8437, 8438, 8439, 8440, 8441, 8442, 8443, 8444, 8445 were selected from the ORFs having the nucleotide sequence represented by SEQ ID NO: 233.

The probes having the nucleotide sequence represented by SEQ ID NOS: 8446, 8447, 8448, 8449, 8450, 8451, 8452, 8453, 8454, 8455, 8456 were selected from the ORFs having the nucleotide sequence represented by SEQ ID NO: 234.

The probes having the nucleotide sequence represented by SEQ ID NOS: 8457, 8458, 8459, 8460, 8461, 8462, 8463, 8464, 8465, 8466, 8467 were selected from the ORFs having the nucleotide sequence represented by SEQ ID NO: 235.

The probes having the nucleotide sequence represented by SEQ ID NOS: 8468, 8469, 8470, 8471, 8472, 8473, 8474, 8475, 8476, 8477, 8478 were selected from the ORFs having the nucleotide sequence represented by SEQ ID NO: 236.

The probes having the nucleotide sequence represented by SEQ ID NOS: 8479, 8480, 8481, 8482, 8483, 8484, 8485, 8486, 8487, 8488, 8489 were selected from the ORFs having the nucleotide sequence represented by SEQ ID NO: 237.

The probes having the nucleotide sequence represented by SEQ ID NOS: 8490, 8491, 8492, 8493, 8494, 8495, 8496, 8497, 8498, 8499, 8500 were selected from the ORFs having the nucleotide sequence represented by SEQ ID NO: 238.

The probes having the nucleotide sequence represented by SEQ ID NOS: 8501, 8502, 8503, 850.4, 8505, 8506, 8507, 8508, 8509, 8510, 8511 were selected from the ORFs having the nucleotide sequence represented by SEQ ID NO: 239.

The probes having the nucleotide sequence represented by SEQ ID NOS: 8512, 8513, 8514, 8515, 8516, 8517, 8518, 8519, 8520, 8521, 8522 were selected from the ORFs having the nucleotide sequence represented by SEQ ID NO: 240.

The probes having the nucleotide sequence represented by SEQ ID NOS: 8523, 8524, 8525, 8526, 8527, 8528, 8529, 8530, 8531, 8532, 8533 were selected from the ORFs having the nucleotide sequence represented by SEQ ID NO: 241.

The probes having the nucleotide sequence represented by SEQ ID NOS: 8534, 8535, 8536, 8537, 8538, 8539, 8540, 8541, 8542, 8543, 8544 were selected from the ORFs having the nucleotide sequence represented by SEQ ID NO: 242.

The probes having the nucleotide sequence represented by SEQ ID NOS: 8545, 8546, 8547, 8548, 8549, 8550, 8551, 8552, 8553, 8554, 8555 were selected from the ORFs having the nucleotide sequence represented by SEQ ID NO: 243.

The probes having the nucleotide sequence represented by SEQ ID NOS: 8556, 8557, 8558, 8559, 8560, 8561, 8562, 8563, 8564, 8565, 8566 were selected from the ORFs having the nucleotide sequence represented by SEQ ID NO: 244.

The probes having the nucleotide sequence represented by SEQ ID NOS: 8567, 8568, 8569, 8570, 8571, 8572, 8573, 8574, 8575, 8576, 8577 were selected from the ORFs having the nucleotide sequence represented by SEQ ID NO: 245.

The probes having the nucleotide sequence represented by SEQ ID NOS: 8578, 8579, 8580, 8581, 8582, 8583, 8584, 8585, 8586, 8587, 8588 were selected from the ORFs having the nucleotide sequence represented by SEQ ID NO: 246.

The probes having the nucleotide sequence represented by SEQ ID NOS: 8589, 8590, 8591, 8592, 8593, 8594, 8595, 8596, 8597, 8598, 8599 were selected from the ORFs having the nucleotide sequence represented by SEQ ID NO: 247.

The probes having the nucleotide sequence represented by SEQ ID NOS: 8600, 8601, 8602, 8603, 8604, 8605, 8606, 8607, 8608, 8609, 8610 were selected from the ORFs having the nucleotide sequence represented by SEQ ID NO: 248.

The probes having the nucleotide sequence represented by SEQ ID NOS: 8611, 8612, 8613, 8614, 8615, 8616, 8617, 8618, 8619, 8620, 8621 were selected from the ORFs having the nucleotide sequence represented by SEQ ID NO: 249.

The probes having the nucleotide sequence represented by SEQ ID NOS: 8622, 8623, 8624, 8625, 8626, 8627, 8628, 8629, 8630, 8631, 8632 were selected from the ORFs having the nucleotide sequence represented by SEQ ID NO: 250.

The probes having the nucleotide sequence represented by SEQ ID NOS: 8633, 8634, 8635, 8636, 8637, 8638, 8639, 8640, 8641, 8642, 8643 were selected from the ORFs having the nucleotide sequence represented by SEQ ID NO: 251.

The probes having the nucleotide sequence represented by SEQ ID NOS: 8644, 8645, 8646, 8647, 8648, 8649, 8650, 8651, 8652, 8653, 8654 were selected from the ORFs having the nucleotide sequence represented by SEQ ID NO: 252.

The probes having the nucleotide sequence represented by SEQ ID NOS: 8655, 8656, 8657, 8658, 8659, 8660, 8661, 8662, 8663, 8664, 8665 were selected from the ORFs having the nucleotide sequence represented by SEQ ID NO: 253.

The probes having the nucleotide sequence represented by SEQ ID NOS: 8666, 8667, 8668, 8669, 8670, 8671, 8672, 8673, 8674, 8675, 8676 were selected from the ORFs having the nucleotide sequence represented by SEQ ID NO: 254.

The probes having the nucleotide sequence represented by SEQ ID NOS: 8677, 8678, 8679, 8680, 8681, 8682, 8683, 8684, 8685, 8686, 8687 were selected from the ORFs having the nucleotide sequence represented by SEQ ID NO: 255.

The probes having the nucleotide sequence represented by SEQ ID NOS: 8688, 8689, 8690, 8691, 8692, 8693, 8694, 8695, 8696, 8697, 8698 were selected from the ORFs having the nucleotide sequence represented by SEQ ID NO: 256.

The probes having the nucleotide sequence represented by SEQ ID NOS: 8699, 8700, 8701, 8702, 8703, 8704, 8705, 8706, 8707, 8708, 8709 were selected from the ORFs having the nucleotide sequence represented by SEQ ID NO: 257.

The probes having the nucleotide sequence represented by SEQ ID NOS: 8710, 8711, 8712, 8713, 8714, 8715, 8716, 8717, 8718, 8719, 8720 were selected from the ORFs having the nucleotide sequence represented by SEQ ID NO: 258.

The probes having the nucleotide sequence represented by SEQ ID NOS: 8721, 8722, 8723, 8724, 8725, 8726, 8727, 8728, 8729, 8730, 8731 were selected from the ORFs having the nucleotide sequence represented by SEQ ID NO: 259.

The probes having the nucleotide sequence represented by SEQ ID NOS: 8732, 8733, 8734, 8735, 8736, 8737, 8738, 8739, 8740, 8741, 8742 were selected from the ORFs having the nucleotide sequence represented by SEQ ID NO: 260.

The probes having the nucleotide sequence represented by SEQ ID NOS: 8743, 8744, 8745, 8746, 8747, 8748, 8749, 8750, 8751, 8752, 8753 were selected from the ORFs having the nucleotide sequence represented by SEQ ID NO: 261.

The probes having the nucleotide sequence represented by SEQ ID NOS: 8754, 8755, 8756, 8757, 8758, 8759, 8760, 8761, 8762, 8763, 8764 were selected from the ORFs having the nucleotide sequence represented by SEQ ID NO: 262.

The probes having the nucleotide sequence represented by SEQ ID NOS: 8765, 8766, 8767, 8768, 8769, 8770, 8771, 8772, 8773, 8774, 8775 were selected from the ORFs having the nucleotide sequence represented by SEQ ID NO: 263.

The probes having the nucleotide sequence represented by SEQ ID NOS: 8776, 8777, 8778, 8779, 8780, 8781, 8782, 8783, 8784, 8785, 8786 were selected from the-ORFs having the nucleotide sequence represented by SEQ ID NO: 264.

The probes having the nucleotide sequence represented by SEQ ID NOS: 8787, 8788, 8789, 8790, 8791, 8792, 8793, 8794, 8795, 8796, 8797 were selected from the ORFs having the nucleotide sequence represented by SEQ ID NO: 265.

The probes having the nucleotide sequence represented by SEQ ID NOS: 8798, 8799, 8800, 8801, 8802, 8803, 8804, 8805, 8806, 8807, 8808 were selected from the ORFs having the nucleotide sequence represented by SEQ ID NO: 266.

The probes having the nucleotide sequence represented by SEQ ID NOS: 8809, 8810, 8811, 8812, 8813, 8814, 8815, 8816, 8817, 8818, 8819 were selected from the ORFs having the nucleotide sequence represented by SEQ ID NO: 267.

The probes having the nucleotide sequence represented by SEQ ID NOS: 8820, 8821, 8822, 8823, 8824, 8825, 8826, 8827, 8828, 8829, 8830 were selected from the ORFs having the nucleotide sequence represented by SEQ ID NO: 268.

The probes having the nucleotide sequence represented by SEQ ID NOS: 8831, 8832, 8833, 8834, 8835, 8836, 8837, 8838, 8839, 8840, 8841 were selected from the ORFs having the nucleotide sequence represented by SEQ ID NO: 269.

The probes having the nucleotide sequence represented by SEQ ID NOS: 8842, 8843, 8844, 8845, 8846, 8847, 8848, 8849, 8850, 8851, 8852 were selected from the ORFs having the nucleotide sequence represented by SEQ ID NO: 270.

The probes having the nucleotide sequence represented by SEQ ID NOS: 8853, 8854, 8855, 8856, 8857, 8858, 8859, 8860, 8861, 8862, 8863 were selected from the ORFs having the nucleotide sequence represented by SEQ ID NO: 271.

The probes having the nucleotide sequence represented by SEQ ID NOS: 8864, 8865, 8866, 8867, 8868, 8869, 8870, 8871, 8872, 8873, 8874 were selected from the ORFs having the nucleotide sequence represented by SEQ ID NO: 272.

The probes having the nucleotide sequence represented by SEQ ID NOS: 8875, 8876, 8877, 8878, 8879, 8880, 8881, 8882, 8883, 8884, 8885 were selected from the ORFs having the nucleotide sequence represented by SEQ ID NO: 273.

The probes having the nucleotide sequence represented by SEQ ID NOS: 8886, 8887, 8888, 8889, 8890, 8891, 8892, 8893, 8894, 8895, 8896 were selected from the ORFs having the nucleotide sequence represented by SEQ ID NO: 274.

The probes having the nucleotide sequence represented by SEQ ID NOS: 8897, 8898, 8899, 8900, 8901, 8902, 8903, 8904, 8905, 8906, 8907 were selected from the ORFs having the nucleotide sequence represented by SEQ ID NO: 275.

The probes having the nucleotide sequence represented by SEQ ID NOS: 8908, 8909, 8910, 8911, 8912, 8913, 8914, 8915, 8916, 8917, 8918 were selected from the ORFs having the nucleotide sequence represented by SEQ ID NO: 276.

The probes having the nucleotide sequence represented by SEQ ID NOS: 8919, 8920, 8921, 8922, 8923, 8924, 8925, 8926, 8927, 8928, 8929 were selected from the ORFs having the nucleotide sequence represented by SEQ ID NO: 277.

The probes having the nucleotide sequence represented by SEQ ID NOS: 8930, 8931, 8932, 8933, 8934, 8935, 8936, 8937, 8938, 8939, 8940 were selected from the ORFs having the nucleotide sequence represented by SEQ ID NO: 278.

The probes having the nucleotide sequence represented by SEQ ID NOS: 8941,. 8942, 8943, 8944, 8945, 8946, 8947, 8948, 8949, 8950, 8951 were selected from the ORFs having the nucleotide sequence represented by SEQ ID NO: 279.

The probes having the nucleotide sequence represented by SEQ ID NOS: 8952, 8953, 8954, 8955, 8956, 8957, 8958, 8959, 8960, 8961, 8962 were selected from the ORFs having the nucleotide sequence represented by SEQ ID NO: 280.

The probes having the nucleotide sequence represented by SEQ ID NOS: 8963, 8964, 8965, 8966, 8967, 8968, 8969, 8970, 8971, 8972, 8973 were selected from the ORFs having the nucleotide sequence represented by SEQ ID NO: 281.

The probes having the nucleotide sequence represented by SEQ ID NOS: 8974, 8975, 8976, 8977, 8978, 8979, 8980, 8981, 8982, 8983, 8984 were selected from the ORFs having the nucleotide sequence represented by SEQ ID NO: 282.

The probes having the nucleotide sequence represented by SEQ ID NOS: 8985, 8986, 8987, 8988, 8989, 8990, 8991, 8992, 8993, 8994, 8995 were selected from the ORFs having the nucleotide sequence represented by SEQ ID NO: 283.

The probes having the nucleotide sequence represented by SEQ ID NOS: 8996, 8997, 8998, 8999, 9000, 9001, 9002, 9003, 9004, 9005, 9006 were selected from the ORFs having the nucleotide sequence represented by SEQ ID NO: 284.

The probes having the nucleotide sequence represented by SEQ ID NOS: 9007, 9008, 9009, 9010, 9011, 9012, 9013, 9014, 9015, 9016, 9017 were selected from the ORFs having the nucleotide sequence represented by SEQ ID NO: 285.

The probes having the nucleotide sequence represented by SEQ ID NOS: 9018, 9019, 9020, 9021, 9022, 9023, 9024, 9025, 9026, 9027, 9028 were selected from the ORFs having the nucleotide sequence represented by SEQ ID NO: 286.

The probes having the nucleotide sequence represented by SEQ ID NOS: 9029, 9030, 9031, 9032, 9033, 9034, 9035, 9036, 9037, 9038, 9039 were selected from the ORFs having the nucleotide sequence represented by SEQ ID NO: 287.

The probes having the nucleotide sequence represented by SEQ. ID NOS: 9040, 9041, 9042, 9043, 9044, 9045, 9046, 9047, 9048, 9049, 9050 were selected from the ORFs having the nucleotide sequence represented by SEQ ID NO: 288.

The probes having the nucleotide sequence represented by SEQ ID NOS: 9051, 9052, 9053, 9054, 9055, 9056, 9057, 9058, 9059, 9060, 9061 were selected from the ORFs having the nucleotide sequence represented by SEQ ID NO: 289.

The probes having the nucleotide sequence represented by SEQ ID NOS: 9062, 9063, 9064, 9065, 9066, 9067, 9068, 9069, 9070, 9071, 9072 were selected from the ORFs having the nucleotide sequence represented by SEQ ID NO: 290.

The probes having the nucleotide sequence represented by SEQ ID NOS: 9073, 9074, 9075, 9076, 9077, 9078, 9079, 9080, 9081, 9082, 9083 were selected from the ORFs having the nucleotide sequence represented by SEQ ID NO: 291.

The probes having the nucleotide sequence represented by SEQ ID NOS: 9084, 9085, 9086, 9087, 9088, 9089, 9090, 9091, 9092, 9093, 9094 were selected from the ORFs having the nucleotide sequence represented by SEQ ID NO: 292.

The probes having the nuclebtide sequence represented by SEQ ID NOS: 9095, 9096, 9097, 9098, 9099, 9100, 9101, 9102, 9103, 9104, 9105 were selected from the ORFs having the nucleotide sequence represented by SEQ ID NO: 293.

The probes having the nucleotide sequence represented by SEQ ID NOS: 9106, 9107, 9108, 9109, 9110, 9111, 9112, 9113, 9114, 9115, 9116 were selected from the ORFs having the nucleotide sequence represented by SEQ ID NO: 294.

The probes having the nucleotide sequence represented by SEQ ID NOS: 9117, 9118, 9119, 9120, 9121, 9122, 9123, 9124, 9125, 9126, 9127 were selected from the ORFs having the nucleotide sequence represented by SEQ ID NO: 295.

The probes having the nucleotide sequence represented by SEQ ID NOS: 9128, 9129, 9130, 9131, 9132, 9133, 9134, 9135, 9136, 9137, 9138 were selected from the ORFs having the nucleotide sequence represented by SEQ ID NO: 296.

The probes having the nucleotide sequence represented by SEQ ID NOS: 9139, 9140, 9141, 9142, 9143, 9144, 9145, 9146, 9147, 9148, 9149 were selected from the ORFs having the nucleotide sequence represented by SEQ ID NO: 297.

The probes having the nucleotide sequence represented by SEQ ID NOS: 9150, 9151, 9152, 9153, 9154, 9155, 9156, 9157, 9158, 9159, 9160 were selected from the ORFs having the nucleotide sequence represented by SEQ ID NO: 298.

The probes having the nucleotide sequence represented by SEQ ID NOS: 9161, 9162, 9163, 9164, 9165, 9166, 9167, 9168, 9169, 9170, 9171 were selected from the ORFs having the nucleotide sequence represented by SEQ ID NO: 299.

The probes having the nucleotide sequence represented by SEQ ID NOS: 9172, 9173, 9174, 9175, 9176, 9177, 9178, 9179, 9180, 9181, 9182 were selected from the ORFs having the nucleotide sequence represented by SEQ ID NO: 300.

The probes having the nucleotide sequence represented by SEQ ID NOS: 9183, 9184, 9185, 9186, 9187, 9188, 9189, 9190, 9191, 9192, 9193 were selected from the ORFs having the nucleotide sequence represented by SEQ ID NO: 301.

The probes having the nucleotide sequence represented by SEQ ID NOS: 9194, 9195, 9196, 9197, 9198, 9199, 9200, 9201, 9202, 9203, 9204 were selected from the ORFs having the nucleotide sequence represented by SEQ ID NO: 302.

The probes having the nucleotide sequence represented by SEQ ID NOS: 9205, 9206, 9207, 9208, 9209, 9210, 9211, 9212, 9213, 9214, 9215 were selected from the ORFs having the nucleotide sequence represented by SEQ ID NO: 303.

The probes having the nucleotide sequence represented by SEQ ID NOS: 9216, 9217, 9218, 9219, 9220, 9221, 9222, 9223, 9224, 9225, 9226 were selected from the ORFs having the nucleotide sequence represented by SEQ ID NO: 304.

The probes having the nucleotide sequence represented by SEQ ID NOS: 9227, 9228, 9229, 9230, 9231, 9232, 9233, 9234, 9235, 9236, 9237 were selected from the ORFs having the nucleotide sequence represented by SEQ ID NO: 305.

The probes having the nucleotide sequence represented by SEQ ID NOS: 9238, 9239, 9240, 9241, 9242, 9243, 9244, 9245, 9246, 9247, 9248 were selected from the ORFs having the nucleotide sequence represented by SEQ ID NO: 306.

The probes having the nucleotide sequence represented by SEQ ID NOS: 9249, 9250, 9251, 9252, 9253, 9254, 9255, 9256, 9257, 9258, 9259 were selected from the ORFs having the nucleotide sequence represented by SEQ ID NO: 307.

The probes having the nucleotide sequence represented by SEQ ID NOS: 9260, 9261, 9262, 9263, 9264, 9265, 9266, 9267, 9268, 9269, 9270 were selected from the ORFs having the nucleotide sequence represented by SEQ ID NO: 308.

The probes having the nucleotide sequence represented by SEQ ID NOS: 9271, 9272, 9273, 9274, 9275, 9276, 9277, 9278, 9279, 9280, 9281 were selected from the ORFs having the nucleotide sequence represented by SEQ ID NO: 309.

The probes having the nucleotide sequence represented by SEQ ID NOS: 9282, 9283, 9284, 9285, 9286, 9287, 9288, 9289, 9290, 9291, 9292 were selected from the ORFs having the nucleotide sequence represented by SEQ ID NO: 310.

The probes having the nucleotide sequence represented by SEQ ID NOS: 9293, 9294, 9295, 9296, 9297, 9298, 9299, 9300, 9301, 9302, 9303 were selected from the ORFs having the nucleotide sequence represented by SEQ ID NO: 311.

The probes having the nucleotide sequence represented by SEQ ID NOS: 9304, 9305, 9306, 9307, 9308, 9309, 9310, 9311, 9312, 9313, 9314 were selected from the ORFs having the nucleotide sequence represented by SEQ ID NO: 312.

The probes having the nucleotide sequence represented by SEQ ID NOS: 9315, 9316, 9317, 9318, 9319, 9320, 9321, 9322, 9323, 9324, 9325 were selected from the ORFs having the nucleotide sequence represented by SEQ ID NO: 313.

The probes having the nucleotide sequence represented by SEQ ID NOS: 9326, 9327, 9328, 9329, 9330, 9331, 9332, 9333, 9334, 9335, 9336 were selected from the ORFs having the nucleotide sequence represented by SEQ ID NO: 314.

The probes having the nucleotide sequence represented by SEQ ID NOS: 9337, 9338, 9339, 9340, 9341, 9342, 9343, 9344, 9345, 9346, 9347, 9348, 9349, 9350, 9351, 9352, 9353, 9354, 9355, 9356, 9357, 9358 were selected from the ORFs having the nucleotide sequence represented by SEQ ID NO: 315.

The probes having the nucleotide sequence represented by SEQ ID NOS: 9359, 9360, 9361, 9362, 9363, 9364, 9365, 9366, 9367, 9368, 9369 were selected from the ORFs having the nucleotide sequence represented by SEQ ID NO: 316.

The probes having the nucleotide sequence represented by SEQ ID NOS: 9370, 9371, 9372, 9373, 9374, 9375, 9376, 9377, 9378, 9379, 9380 were selected from the ORFs having the nucleotide sequence represented by SEQ ID NO: 317.

The probes having the nucleotide sequence represented by SEQ ID NOS: 9381, 9382, 9383, 9384, 9385, 9386, 9387, 9388, 9389, 9390, 9391 were selected from the ORFs having the nucleotide sequence represented by SEQ ID NO: 318.

The probes having the nucleotide sequence represented by SEQ ID NOS: 9392, 9393, 9394, 9395, 9396, 9397, 9398, 9399, 9400, 9401, 9402 were selected from the ORFs having the nucleotide sequence represented by SEQ ID NO: 319.

The probes having the nucleotide sequence represented by SEQ ID NOS: 9403, 9404, 9405, 9406, 9407, 9408, 9409, 9410, 9411, 9412, 9413 were selected from the ORFs having the nucleotide sequence represented by SEQ ID NO: 320.

The probes having the nucleotide sequence represented by SEQ ID NOS: 9414, 9415, 9416, 9417, 9418, 9419, 9420, 9421, 9422, 9423, 9424 were selected from the ORFs having the nucleotide sequence represented by SEQ ID NO: 321.

The probes having the nucleotide sequence represented by SEQ ID NOS: 9425, 9426, 9427, 9428, 9429, 9430, 9431, 9432, 9433, 9434, 9435 were selected from the ORFs having the nucleotide sequence represented by SEQ ID NO: 322.

The probes having the nucleotide sequence represented by SEQ ID NOS: 9436, 9437, 9438, 9439, 9440, 9441, 9442, 9443, 9444, 9445, 9446 were selected from the ORFs having the nucleotide sequence represented by SEQ ID NO: 323.

The probes having the nucleotide sequence represented by SEQ ID NOS: 9447, 9448, 9449, 9450, 9451, 9452, 9453, 9454, 9455, 9456, 9457 were selected from the ORFs having the nucleotide sequence represented by SEQ ID NO: 324.

The probes having the nucleotide sequence represented by SEQ ID NOS: 9458, 9459, 9460, 9461, 9462, 9463, 9464, 9465, 9466, 9467, 9468 were selected from the ORFs having the nucleotide sequence represented by SEQ ID NO: 325.

The probes having the nucleotide sequence represented by SEQ ID NOS: 9469, 9470, 9471, 9472, 9473, 9474, 9475, 9476, 9477, 9478, 9479 were selected from the ORFs having the nucleotide sequence represented by SEQ ID NO: 326.

The probes having the nucleotide sequence represented by SEQ ID NOS: 9480, 9481, 9482, 9483, 9484, 9485, 9486, 9487, 9488, 9489, 9490 were selected from the ORFs having the nucleotide sequence represented by SEQ ID NO: 327.

The probes having the nucleotide sequence represented by SEQ ID NOS: 9491, 9492, 9493, 9494, 9495, 9496, 9497, 9498, 9499, 9500, 9501 were selected from the ORFs having the nucleotide sequence represented by SEQ ID NO: 328.

The probes having the nucleotide sequence represented by SEQ ID NOS: 9502, 9503, 9504, 9505, 9506, 9507, 9508, 9509, 9510, 9511, 9512 were selected from the ORFs having the nucleotide sequence represented by SEQ ID NO: 329.

The probes having the nucleotide sequence represented by SEQ ID NOS: 9513, 9514, 9515, 9516, 9517, 9518, 9519, 9520, 9521, 9522, 9523 were selected from the ORFs having the nucleotide sequence represented by SEQ ID NO: 330.

The probes having the nucleotide sequence represented by SEQ ID NOS: 9524, 9525, 9526, 9527, 9528, 9529, 9530, 9531, 9532, 9533, 9534 were selected from the ORFs having the nucleotide sequence represented by SEQ ID NO: 331.

The probes having the nucleotide sequence represented by SEQ ID NOS: 9535, 9536, 9537, 9538, 9539, 9540, 9541, 9542, 9543, 9544, 9545, 9546, 9547, 9548, 9549, 9550, 9551, 9552, 9553, 9554, 9555, 9556 were selected from the ORFs having the nucleotide sequence represented by SEQ ID NO: 332.

The probes having the nucleotide sequence represented by SEQ ID NOS: 9557, 9558, 9559, 9560, 9561, 9562, 9563, 9564, 9565, 9566, 9567, 9568, 9569, 9570, 9571, 9572, 9573, 9574, 9575, 9576, 9577, 9578 were selected from the ORFs having the nucleotide sequence represented by SEQ ID NO: 333.

The probes having the nucleotide sequence represented by SEQ ID NOS: 9579, 9580, 9581, 9582, 9583, 9584, 9585, 9586, 9587, 9588, 9589 were selected from the ORFs having the nucleotide sequence represented by SEQ ID NO: 334.

The probes having the nucleotide sequence represented by SEQ ID NOS: 9590, 9591, 9592, 9593, 9594, 9595, 9596, 9597, 9598, 9599, 9600 were selected from the ORFs having the nucleotide sequence represented by SEQ ID NO: 335.

The probes having the nucleotide sequence represented by SEQ ID NOS: 9601, 9602, 9603, 9604, 9605, 9606, 9607, 9608, 9609, 9610, 9611 were selected from the ORFs having the nucleotide sequence represented by SEQ ID NO: 336.

The probes having the nucleotide sequence represented by SEQ ID NOS: 9612, 9613, 9614, 9615, 9616, 9617, 9618, 9619, 9620, 9621, 9622 were selected from the ORFs having the nucleotide sequence represented by SEQ ID NO: 337.

The probes having the nucleotide sequence represented by SEQ ID NOS: 9623, 9624, 9625, 9626, 9627, 9628, 9629, 9630, 9631, 9632, 9633 were selected from the ORFs having the nucleotide sequence represented by SEQ ID NO: 338.

The probes having the nucleotide sequence represented by SEQ ID NOS: 9634, 9635, 9636, 9637, 9638, 9639, 9640, 9641, 9642, 9643, 9644 were selected from the ORFs having the nucleotide sequence represented by SEQ ID NO: 339.

The probes having the nucleotide sequence represented by SEQ ID NOS: 9645, 9646, 9647, 9648, 9649, 9650, 9651, 9652, 9653, 9654, 9655 were selected from the ORFs having the nucleotide sequence represented by SEQ ID NO: 340.

The probes having the nucleotide sequence represented by SEQ ID NOS: 9656, 9657, 9658, 9659, 9660, 9661, 9662, 9663, 9664, 9665, 9666 were selected from the ORFs having the nucleotide sequence represented by SEQ ID NO: 341.

The probes having the nucleotide sequence represented by SEQ ID NOS: 9667, 9668, 9669, 9670, 9671, 9672, 9673, 9674, 9675, 9676, 9677 were selected from the ORFs having the nucleotide sequence represented by SEQ ID NO: 342.

The probes having the nucleotide sequence represented-by SEQ ID NOS: 9678, 9679, 9680, 9681, 9682, 9683, 9684, 9685, 9686, 9687, 9688 were selected from the ORFs having the nucleotide sequence represented by SEQ ID NO: 343.

The probes having the nucleotide sequence-represented by SEQ ID NOS: 9689, 9690, 9691, 9692, 9693, 9694, 9695, 9696, 9697, 9698, 9699 were selected from the ORFs having the nucleotide sequence represented by SEQ ID NO: 344.

The probes having-the nucleotide sequence represented by SEQ ID NOS: 9700, 9701, 9702, 9703, 9704, 9705, 9706, 9707, 9708, 9709, 9710 were selected from the ORFs having the nucleotide sequence represented by SEQ ID NO: 345.

The probes having the nucleotide sequence represented by SEQ ID NOS: 9711, 9712, 9713, 9714, 9715, 9716, 9717, 9718, 9719, 9720, 9721 were selected from the ORFs having the nucleotide sequence represented by SEQ ID NO: 346.

The probes having the nucleotide sequence represented by SEQ ID NOS: 9722, 9723, 9724, 9725, 9726, 9727, 9728, 9729, 9730, 9731, 9732 were selected from the ORFs having the nucleotide sequence represented by SEQ ID NO: 347.

The probes having the nucleotide sequence represented by SEQ ID NOS: 9733, 9734, 9735, 9736, 9737, 9738, 9739, 9740, 9741, 9742, 9743 were selected from the ORFs having the nucleotide sequence represented by SEQ ID NO: 348.

The probes having the nucleotide sequence represented by SEQ ID NOS: 9744, 9745, 9746, 9747, 9748, 9749, 9750, 9751, 9752, 9753, 9754 were selected from the ORFs having the nucleotide sequence represented by SEQ ID NO: 349.

The probes having the nucleotide sequence represented by SEQ ID NOS: 9755, 9756, 9757, 9758, 9759, 9760, 9761, 9762, 9763, 9764, 9765 were selected from the ORFs having the nucleotide sequence represented by SEQ ID NO: 350.

The probes having the nucleotide sequence represented by SEQ ID NOS: 9766, 9767, 9768, 9769, 9770, 9771, 9772, 9773, 9774, 9775, 9776 were selected from the ORFs having the nucleotide sequence represented by SEQ ID NO: 351.

The probes having the nucleotide sequence represented by SEQ ID NOS: 9777, 9778, 9779, 9780, 9781, 9782, 9783, 9784, 9785, 9786, 9787 were selected from the ORFs having the nucleotide sequence represented by SEQ ID NO: 352.

The probes having the nucleotide sequence represented by SEQ ID NOS: 9788, 9789, 9790, 9791, 9792, 9793, 9794, 9795, 9796, 9797, 9798 were selected from the ORFs having the nucleotide sequence represented by SEQ ID NO: 353.

The probes having the nucleotide sequence represented by SEQ ID NOS: 9799, 9800, 9801, 9802, 9803, 9804, 9805, 9806, 9807, 9808, 9809 were selected from the ORFs having the nucleotide sequence represented by SEQ ID NO: 354.

The probes having the nucleotide sequence represented by SEQ ID NOS: 9810, 9811, 9812, 9813, 9814, 9815, 9816, 9817, 9818, 9819, 9820 were selected from the ORFs having the nucleotide sequence represented by SEQ ID NO: 355.

The probes having the nucleotide sequence represented by SEQ ID NOS: 9821, 9822, 9823, 9824, 9825, 9826, 9827, 9828, 9829, 9830, 9831 were selected from the ORFs having the nucleotide sequence represented by SEQ ID NO: 356.

The probes having the nucleotide sequence represented by SEQ ID NOS: 9832, 9833, 9834, 9835, 9836, 9837, 9838, 9839, 9840, 9841, 9842 were selected from the ORFs having the nucleotide sequence represented by SEQ ID NO: 357.

The probes having the nucleotide sequence represented by SEQ ID NOS: 9843, 9844, 9845, 9846, 9847, 9848, 9849, 9850, 9851, 9852, 9853 were selected from the ORFs having the nucleotide sequence represented by SEQ ID NO: 358.

The probes having the nucleotide sequence represented by SEQ ID NOS: 9854, 9855, 9856, 9857, 9858, 9859, 9860, 9861, 9862, 9863, 9864 were selected from the ORFs having the nucleotide sequence represented by SEQ ID NO: 359.

The probes having the nucleotide sequence represented by SEQ ID NOS: 9865, 9866, 9867, 9868, 9869, 9870, 9871, 9872, 9873, 9874, 9875 were selected from the ORFs having the nucleotide sequence represented by SEQ ID NO: 360.

The probes having the nucleotide sequence represented by SEQ ID NOS: 9876, 9877, 9878, 9879, 9880, 9881, 9882, 9883, 9884, 9885, 9886 were selected from the ORFs having the nucleotide sequence represented by SEQ ID NO: 361.

The probes having the nucleotide sequence represented by SEQ ID NOS: 9887, 9888, 9889, 9890, 9891, 9892, 9893, 9894, 9895, 9896, 9897 were selected from the ORFs having the nucleotide sequence represented by SEQ ID NO: 362.

The probes having the nucleotide sequence represented by SEQ ID NOS: 9898, 9899, 9900, 9901, 9902, 9903, 9904, 9905, 9906, 9907, 9908 were selected from the ORFs having the nucleotide sequence represented by SEQ ID NO: 363.

The probes having the nucleotide sequence represented by SEQ ID NOS: 9909, 9910, 9911, 9912, 9913, 9914, 9915, 9916, 9917, 9918, 9919 were selected from the ORFs having the nucleotide sequence represented by SEQ ID NO: 364.

The probes having the nucleotide sequence represented by SEQ ID NOS: 9920, 9921, 9922, 9923, 9924, 9925, 9926, 9927, 9928, 9929, 9930 were selected from the ORFs having the nucleotide sequence represented by SEQ ID NO: 365.

The probes having the nucleotide sequence represented by SEQ ID NOS: 9931, 9932, 9933, 9934, 9935, 9936, 9937, 9938, 9939, 9940, 9941 were selected from the ORFs having the nucleotide sequence represented by SEQ ID NO: 366.

The probes having the nucleotide sequence represented by SEQ ID NOS: 9942, 9943, 9944, 9945, 9946, 9947, 9948, 9949, 9950, 9951, 9952 were selected from the ORFs having the nucleotide sequence represented by SEQ ID NO: 367.

The probes having the nucleotide sequence represented by SEQ ID NOS: 9953, 9954, 9955, 9956, 9957, 9958, 9959, 9960, 9961, 9962, 9963 were selected from the ORFs having the nucleotide sequence represented by SEQ ID NO: 368.

The probes having the nucleotide sequence represented by SEQ ID NOS: 9964, 9965, 9966, 9967, 9968, 9969, 9970, 9971, 9972, 9973, 9974 were selected from the ORFs having the nucleotide sequence represented by SEQ ID NO: 369.

The probes having the nucleotide sequence represented by SEQ ID NOS: 9975, 9976, 9977, 9978, 9979, 9980, 9981, 9982, 9983, 9984, 9985 were selected from the ORFs having the nucleotide sequence represented by SEQ ID NO: 370.

The probes having the nucleotide sequence represented by SEQ ID NOS: 9986, 9987, 9988, 9989, 9990, 9991, 9992, 9993, 9994, 9995, 9996 were selected from the ORFs having the nucleotide sequence represented by SEQ ID NO: 371.

The probes having the nucleotide sequence represented by SEQ ID NOS: 9997, 9998, 9999, 10000, 10001, 10002, 10003, 10004, 10005, 10006, 10007 were selected from the ORFs having the nucleotide sequence represented by SEQ ID NO: 372.

The probes having the nucleotide sequence represented by SEQ ID NOS: 10008, 10009, 10010, 10011, 10012, 10013, 10014, 10015, 10016, 10017, 10018 were selected from the ORFs having the nucleotide sequence represented by SEQ ID NO: 373.

The probes having the nucleotide sequence represented by SEQ ID NOS: 10019, 10020, 10021, 10022, 10023, 10024, 10025, 10026, 10027, 10028, 10029 were selected from the ORFs having the nucleotide sequence represented by SEQ ID NO: 374.

The probes having the nucleotide sequence represented by SEQ ID NOS: 10030, 10031, 10032, 10033, 10034, 10035, 10036, 10037, 10038, 10039, 10040 were selected from the ORFs having the nucleotide sequence represented by SEQ ID NO: 375.

The probes having the nucleotide sequence represented by SEQ ID NOS: 10041, 10042, 10043, 10044, 10045, 10046, 10047, 10048, 10049, 10050, 10051 were selected from the ORFs having the nucleotide sequence represented by SEQ ID NO: 376.

The probes having the nucleotide sequence represented by SEQ ID NOS: 10052, 10053, 10054, 10055, 10056, 10057, 10058, 10059, 10060, 10061, 10062 were selected from the ORFs having the nucleotide sequence represented by SEQ ID NO: 377.

The probes having the nucleotide sequence represented by SEQ ID NOS: 10063, 10064, 10065, 10066, 10067, 10068, 10069, 10070, 10071, 10072, 10073 were selected from the ORFs having the nucleotide sequence represented by SEQ ID NO: 378.

The probes having the nucleotide sequence represented by SEQ ID NOS: 10074, 10075, 10076, 10077, 10078, 10079, 10080, 10081, 10082, 10083, 10084 were selected from the ORFs having the nucleotide sequence represented by SEQ ID NO: 379.

The probes having the nucleotide sequence represented by SEQ ID NOS: 10085, 10086, 10087, 10088, 10089, 10090, 10091, 10092, 10093; 10094, 10095 were selected from the ORFs having the nucleotide sequence represented by SEQ ID NO: 380.

The probes having the nucleotide sequence represented by SEQ ID NOS: 10096, 10097, 10098, 10099, 10100, 10101, 10102, 10103, 10104, 10105, 10106 were selected from the ORFs having the nucleotide sequence represented by SEQ ID NO: 381.

The probes having the nucleotide sequence represented by SEQ ID NOS: 10107, 10108, 10109, 10110, 10111, 10112, 10113, 10114, 10115, 10116, 10117 were selected from the ORFs having the nucleotide sequence represented by SEQ ID NO: 382.

The probes having the nucleotide sequence represented by SEQ ID NOS: 10118, 10119, 10120, 10121, 10122, 10123, 10124, 10125, 10126, 10127, 10128 were selected from the ORFs having the nucleotide sequence represented by SEQ ID NO: 383.

The probes having the nucleotide sequence represented by SEQ ID NOS: 10129, 10130, 10131, 10132, 10133, 10134, 10135, 10136, 10137, 10138, 10139 were selected from the ORFs having the nucleotide sequence represented by SEQ ID NO: 384.

The probes having the nucleotide sequence represented by SEQ ID NOS: 10140, 10141, 10142, 10143, 10144, 10145, 10146, 10147, 10148, 10149, 10150 were selected from the ORFs having the nucleotide sequence represented by SEQ ID NO: 385.

The probes having the nucleotide sequence represented by SEQ ID NOS: 10151, 10152, 10153, 10154, 10155, 10156, 10157, 10158, 10159, 10160, 10161 were selected from the ORFs having the nucleotide sequence represented by SEQ ID NO: 386.

The probes having the nucleotide sequence represented by SEQ ID NOS: 10162, 10163, 10164, 10165, 10166, 10167, 10168, 10169, 10170, 10171, 10172 were selected from the ORFs having the nucleotide sequence represented by SEQ ID NO: 387.

The probes having the nucleotide sequence represented by SEQ ID NOS: 10173, 10174, 10175, 10176, 10177, 10178, 10179, 10180, 10181, 10182, 10183 were selected from the ORFs having the nucleotide sequence represented by SEQ ID NO: 388.

The probes having the nucleotide sequence represented by SEQ ID NOS: 10184, 10185, 10186, 10187, 10188, 10189, 10190, 10191, 10192, 10193, 10194 were selected from the ORFs having the nucleotide sequence represented by SEQ ID NO: 389.

The probes having the nucleotide sequence represented by SEQ ID NOS: 10195, 10196, 10197, 10198, 10199, 10200, 10201, 10202, 10203, 10204, 10205 were selected from the ORFs having the nucleotide sequence represented by SEQ ID NO: 390.

The probes having the nucleotide sequence represented by SEQ ID NOS: 10206, 10207, 10208, 10209, 10210, 10211, 10212, 10213, 10214, 10215, 10216 were selected from the ORFs having the nucleotide sequence represented by SEQ ID NO: 391.

The probes having the nucleotide sequence represented by SEQ ID NOS: 10217, 10218, 10219, 10220, 10221, 10222, 10223, 10224, 10225, 10226, 10227 were selected from the ORFs having the nucleotide sequence represented by SEQ ID NO: 392.

The probes having the nucleotide sequence represented by SEQ ID NOS: 10228, 10229, 10230, 10231, 10232, 10233, 10234, 10235, 10236, 10237, 10238 were selected from the ORFs having the nucleotide sequence represented by SEQ ID NO: 393.

The probes having the nucleotide sequence represented by SEQ ID NOS: 10239, 10240, 10241, 10242, 10243, 10244, 10245, 10246, 10247, 10248, 10249 were selected from the ORFs having the nucleotide sequence represented by SEQ ID NO: 394.

The probes having the nucleotide sequence represented by SEQ ID NOS: 10250, 10251, 10252, 10253, 10254, 10255, 10256, 10257, 10258, 10259, 10260 were selected from the ORFs having the nucleotide sequence represented by SEQ ID NO: 395.

The probes having the nucleotide sequence represented by SEQ ID NOS: 10261, 10262, 10263, 10264, 10265, 10266, 10267, 10268, 10269, 10270, 10271 were selected from the ORFs having the nucleotide sequence represented by SEQ ID NO: 396.

The probes having the nucleotide sequence represented by SEQ ID NOS: 10272, 10273, 10274, 10275, 10276, 10277, 10278, 10279, 10280, 10281, 10282 were selected from the ORFs having the nucleotide sequence represented by SEQ ID NO: 397.

The probes having the nucleotide sequence represented by SEQ ID NOS: 10283, 10284, 10285, 10286, 10287, 10288, 10289, 10290, 10291, 10292, 10293 were selected from the ORFs having the nucleotide sequence represented by SEQ ID NO: 398.

The probes having the nucleotide sequence represented by SEQ ID NOS: 10294, 10295, 10296, 10297, 10298, 10299, 10300, 10301, 10302, 10303, 10304 were selected from the ORFs having the nucleotide sequence represented by SEQ ID NO: 399.

The probes having the nucleotide sequence represented by SEQ ID NOS: 10305, 10306, 10307, 10308, 10309, 10310, 10311, 10312, 10313, 10314, 10315 were selected from the ORFs having the nucleotide sequence represented by SEQ ID NO: 400.

The probes having the nucleotide sequence represented by SEQ ID NOS: 10316, 10317, 10318, 10319, 10320, 10321, 1032.2, 10323, 10324, 10325, 10326 were selected from the ORFs having the nucleotide sequence represented by SEQ ID NO: 401.

The probes having the nucleotide sequence represented by SEQ ID NOS: 10327, 10328, 10329, 10330, 10331, 10332, 10333, 10334, 10335, 10336, 10337 were selected from the ORFs having the nucleotide sequence represented by SEQ ID NO: 402.

The probes having the nucleotide sequence represented by SEQ ID NOS: 10338, 10339, 10340, 10341, 10342, 10343, 10344, 10345, 10346, 10347, 10348 were selected from the ORFs having the nucleotide sequence represented by SEQ ID NO: 403.

The probes having the nucleotide sequence represented by SEQ ID NOS: 10349, 10350, 10351, 10352, 10353, 10354, 10355, 10356, 10357, 10358, 10359 were selected from the ORFs having the nucleotide sequence represented by SEQ ID NO: 404.

The probes having the nucleotide sequence represented by SEQ ID NOS: 10360, 10361, 10362, 10363, 10364, 10365, 10366, 10367, 10368, 10369, 10370 were selected from the ORFs having the nucleotide sequence represented by SEQ ID NO: 405.

The probes having the nucleotide sequence represented by SEQ ID NOS: 10371, 10372, 10373, 10374, 10375, 10376, 10377, 10378, 10379, 10380, 10381 were selected from the ORFs having the nucleotide sequence represented by SEQ ID NO: 406.

The probes having the nucleotide sequence represented by SEQ ID NOS: 10382, 10383, 10384, 10385, 10386, 10387, 10388, 10389, 10390, 10391, 10392 were selected from the ORFs having the nucleotide-sequence represented by SEQ ID NO: 407.

The probes having the nucleotide sequence represented by SEQ ID NOS: 10393, 10394, 10395, 10396, 10397, 10398, 10399, 10400, 10401, 10402, 10403 were selected from the ORFs having the nucleotide sequence represented by SEQ ID NO: 408.

The probes having the nucleotide sequence represented by SEQ ID NOS: 10404, 10405, 10406, 10407, 10408, 10409, 10410, 10411, 10412, 10413, 10414 were selected from the ORFs having the nucleotide sequence represented by SEQ ID NO: 409.

The probes having the nucleotide sequence represented by SEQ ID NOS: 10415, 10416, 10417, 10418, 10419, 10420, 10421, 10422, 10423, 10424, 10425 were selected from the ORFs having the nucleotide sequence represented by SEQ ID NO: 410.

The probes having the nucleotide sequence represented by SEQ ID NOS: 10426, 10427, 10428, 10429, 10430, 10431, 10432, 10433, 10434, 10435, 10436 were selected from the ORFs having the nucleotide sequence represented by SEQ ID NO: 411.

The probes having the nucleotide sequence represented by SEQ ID NOS: 10437, 10438, 10439, 10440, 10441, 10442, 10443, 10444, 10445, 10446, 10447 were selected from the ORFs having the nucleotide sequence represented by SEQ ID NO: 412.

The probes having the nucleotide sequence represented by SEQ ID NOS: 10448, 10449, 10450, 10451, 10452, 10453, 10454, 10455, 10456, 10457, 10458 were selected from the ORFs having the nucleotide sequence represented by SEQ ID NO: 413.

The probes having the nucleotide sequence represented by SEQ ID NOS: 10459, 10460, 10461, 10462, 10463, 10464, 10465, 10466, 10467, 10468, 10469 were selected from the ORFs having the nucleotide sequence represented by SEQ ID NO: 414.

The probes having the nucleotide sequence represented by SEQ ID NOS: 10470, 10471, 10472, 10473, 10474, 10475, 10476, 10477, 10478, 10479, 10480 were selected from the ORFs having the nucleotide sequence represented by SEQ ID NO: 415.

The probes having the nucleotide sequence represented by SEQ ID NOS: 10481, 10482, 10483, 10484, 10485, 10486, 10487, 10488, 10489, 10490, 10491 were selected from the ORFs having the nucleotide sequence represented by SEQ ID NO: 416.

The probes having the nucleotide sequence represented by SEQ ID NOS: 10492, 10493, 10494, 10495, 10496, 10497, 10498, 10499, 10500, 10501, 10502 were selected from the ORFs having the nucleotide sequence represented by SEQ ID NO: 417.

The probes having the nucleotide sequence represented by SEQ ID NOS: 10503, 10504, 10505, 10506, 10507, 10508, 10509, 10510, 10511, 10512, 10513 were selected from the ORFs having the nucleotide sequence represented by SEQ ID NO: 418.

The probes having the nucleotide sequence represented by SEQ ID NOS: 10514, 10515, 10516, 10517, 10518, 10519, 10520, 10521, 10522, 10523, 10524 were selected from the ORFs having the nucleotide sequence represented by SEQ ID NO: 419.

The probes having the nucleotide sequence represented by SEQ ID NOS: 10525, 10526, 10527, 10528, 10529, 10530, 10531, 10532, 10533, 10534, 10535 were selected from the ORFs having the nucleotide sequence represented by SEQ ID NO: 420.

The probes having the nucleotide sequence represented by SEQ ID NOS: 10536, 10537, 10538, 10539, 10540, 10541, 10542, 10543, 10544, 10545, 10546 were selected from the ORFs having the nucleotide sequence represented by SEQ ID NO: 421.

The probes having the nucleotide sequence represented by SEQ ID NOS: 10547, 10548, 10549, 10550, 10551, 10552, 10553, 10554, 10555, 10556, 10557 were selected from the ORFs having the nucleotide sequence represented by SEQ ID NO: 422.

The probes having the nucleotide sequence represented by SEQ ID NOS: 10558, 10559, 10560, 10561, 10562, 10563, 10564, 10565, 10566, 10567, 10568 were selected from the ORFs having the nucleotide sequence represented by SEQ ID NO: 423.

The probes having the nucleotide sequence represented by SEQ ID NOS: 10569, 10570, 10571, 10572, 10573, 10574, 10575, 10576, 10577, 10578, 10579 were selected from the ORFs having the nucleotide sequence represented by SEQ ID NO: 424.

The probes having the nucleotide sequence represented by SEQ ID NOS: 10580, 10581, 10582, 10583, 10584, 10585, 10586, 10587, 10588, 10589, 10590 were selected from the ORFs having the nucleotide sequence represented by SEQ ID NO: 425.

The probes having the nucleotide sequence represented by SEQ ID NOS: 10591, 10592, 10593, 10594, 10595, 10596, 10597, 10598, 10599, 10600, 10601 were selected from the ORFs having the nucleotide sequence represented by SEQ ID NO: 426.

The probes having the nucleotide sequence represented by SEQ ID NOS: 10602, 10603, 10604, 10605, 10606, 10607, 10608, 10609, 10610, 10611, 10612 were selected from the ORFs having the nucleotide sequence represented by SEQ ID NO: 427.

The probes having the nucleotide sequence represented by SEQ ID NOS: 10613, 10614, 10615, 10616, 10617, 10618, 10619, 10620, 10621, 10622, 10623 were selected from the ORFs having the nucleotide sequence represented by SEQ ID NO: 428.

The probes having the nucleotide sequence represented by SEQ ID NOS: 10624, 10625, 10626, 10627, 10628, 10629, 10630, 10631, 10632, 10633, 10634 were selected from the ORFs having the nucleotide sequence represented by SEQ ID NO: 429.

The probes having the nucleotide sequence represented by SEQ ID NOS: 10635, 10636, 10637, 10638, 10639, 10640, 10641, 10642, 10643, 10644, 10645 were selected from the ORFs having the nucleotide sequence represented by SEQ ID NO: 430.

The probes having the nucleotide sequence represented by SEQ ID NOS: 10646, 10647, 10648, 10649, 10650, 10651, 10652, 10653, 10654, 10655, 10656 were selected from the ORFs having the nucleotide sequence represented by SEQ ID NO: 431.

The probes having the nucleotide sequence represented by SEQ ID NOS: 10657, 10658, 10659, 10660, 10661, 10662, 10663, 10664, 10665, 10666, 10667 were selected from the ORFs having the nucleotide sequence represented by SEQ ID NO: 432.

The probes having the nucleotide sequence represented by SEQ ID NOS: 10668, 10669, 10670, 10671, 10672, 10673, 10674, 10675, 10676, 10677, 10678 were selected from the ORFs having the nucleotide sequence represented by SEQ ID NO: 433.

The probes having the nucleotide sequence represented by SEQ ID NOS: 10679, 10680, 10681, 10682, 10683, 10684, 10685, 10686, 10687, 10688, 10689 were selected from the ORFs having the nucleotide sequence represented by SEQ ID NO: 434.

The probes having the nucleotide sequence represented by SEQ ID NOS: 10690, 10691, 10692, 10693, 10694, 10695, 10696, 10697, 10698, 10699, 10700 were selected from the ORFs having the nucleotide sequence represented by SEQ ID NO: 435.

The probes having the nucleotide sequence represented by SEQ ID NOS: 10701, 10702, 10703, 10704, 10705, 10706, 10707, 10708, 10709, 10710, 10711 were selected from the ORFs having the nucleotide sequence represented by SEQ ID NO: 436.

The probes having the nucleotide sequence represented by SEQ ID NOS: 10712, 10713, 10714, 10715, 10716, 10717, 10718, 10719, 10720, 10721, 10722 were selected from the ORFs having the nucleotide sequence represented by SEQ ID NO: 437.

The probes having the nucleotide sequence represented by SEQ ID NOS: 10723, 10724, 10725, 10726, 10727, 10728, 10729, 10730, 10731, 10732, 10733 were selected from the ORFs having the nucleotide sequence represented by SEQ ID NO: 438.

The probes having the nucleotide sequence represented by SEQ ID NOS: 10734, 10735, 10736, 10737, 10738, 10739, 10740, 10741, 10742, 10743, 10744 were selected from the ORFs having the nucleotide sequence represented by SEQ ID NO: 439.

The probes having the nucleotide sequence represented by SEQ ID NOS: 10745, 10746, 10747, 10748, 10749, 10750, 10751, 10752, 10753, 10754, 10755 were selected from the ORFs having the nucleotide sequence represented by SEQ ID NO: 440.

The probes having the nucleotide sequence represented by SEQ ID NOS: 10756, 10757, 10758, 10759, 10760, 10761, 10762, 10763, 10764, 10765, 10766 were selected from the ORFs having the nucleotide sequence represented by SEQ ID NO: 441.

The probes having the nucleotide sequence represented by SEQ ID NOS: 10767, 10768, 10769, 10770, 10771, 10772, 10773, 10774, 10775, 10776, 10777 were selected from the ORFs having the nucleotide sequence represented by SEQ ID NO: 442.

The probes having the nucleotide sequence represented by SEQ ID NOS: 10778, 10779, 10780, 10781, 10782, 10783, 10784, 10785, 10786, 10787, 10788 were selected from the ORFs having the nucleotide sequence represented by SEQ ID NO: 443.

The probes having the nucleotide sequence represented by SEQ ID NOS: 10789, 10790, 10791, 10792, 10793, 10794, 10795, 10796, 10797, 10798, 10799 were selected from the ORFs having the nucleotide sequence represented by SEQ ID NO: 444.

The probes having the nucleotide sequence represented by SEQ ID NOS: 10800, 10801, 10802, 10803, 10804, 10805, 10806, 10807, 10808, 10809, 10810 were selected from the ORFs having the nucleotide sequence represented by SEQ ID NO: 445.

The probes having the nucleotide sequence represented by SEQ ID NOS: 10811, 10812, 10813, 10814, 10815, 10816, 10817, 10818, 10819, 10820, 10821 were selected from the ORFs having the nucleotide sequence represented by SEQ ID NO: 446.

The probes having the nucleotide sequence represented by SEQ ID NOS: 10822, 10823, 10824, 10825, 10826, 10827, 10828, 10829, 10830, 10831, 10832 were selected from the ORFs having the nucleotide sequence represented by SEQ ID NO: 447.

The probes having the nucleotide sequence represented by SEQ ID NOS: 10833, 10834, 10835, 10836, 10837, 10838, 10839, 10840, 10841, 10842, 10843 were selected from the ORFs having the nucleotide sequence represented by SEQ ID NO: 448.

The probes having the nucleotide sequence represented by SEQ ID NOS: 10844, 10845, 10846, 10847, 10848, 10849, 10850, 10851, 10852, 10853, 10854 were selected from the ORFs having the nucleotide sequence represented by SEQ ID NO: 449.

The probes having the nucleotide sequence represented by SEQ ID NOS: 10855, 10856, 10857, 10858, 10859, 10860, 10861, 10862, 10863, 10864, 10865 were selected from the ORFs having the nucleotide sequence represented by SEQ ID NO: 450.

The probes having the nucleotide sequence represented by SEQ ID NOS: 10866, 10867, 10868, 10869, 10870, 10871, 10872, 10873, 10874, 10875, 10876 were selected from the ORFs having the nucleotide sequence represented by SEQ ID NO: 451.

The probes having the nucleotide sequence represented by SEQ ID NOS: 10877, 10878, 10879, 10880, 10881, 10882, 10883, 10884, 10885, 10886, 10887 were selected from the ORFs having the nucleotide sequence represented by SEQ ID NO: 452.

The probes having the nucleotide sequence represented by SEQ ID NOS: 10888, 10889, 10890, 10891, 10892, 10893, 10894, 10895, 10896, 10897, 10898 were selected from the ORFs having the nucleotide sequence represented by SEQ ID NO: 453.

The probes having the nucleotide sequence represented by SEQ ID NOS: 10899, 10900, 10901, 10902, 10903, 10904, 10905, 10906, 10907, 10908, 10909 were selected from the ORFs having the nucleotide sequence represented by SEQ ID NO: 454.

The probes having the nucleotide sequence represented by SEQ ID NOS: 10910, 10911, 10912, 10913, 10914, 10915, 10916, 10917, 10918, 10919, 10920 were selected from the ORFs having the nucleotide sequence represented by SEQ ID NO: 455.

The probes having the nucleotide sequence represented by SEQ ID NOS: 10921, 10922, 10923, 10924, 10925, 10926, 10927, 10928, 10929, 10930, 10931 were selected from the ORFs having the nucleotide sequence represented by SEQ ID NO: 456.

The probes having the nucleotide sequence represented by SEQ ID NOS: 10932, 10933, 10934, 10935, 10936, 10937, 10938, 10939, 10940, 10941, 10942 were selected from the ORFs having the nucleotide sequence represented by SEQ ID NO: 457.

The probes having the nucleotide sequence represented by SEQ ID NOS: 10943, 10944, 10945, 10946, 10947, 10948, 10949, 10950, 10951, 10952, 10953 were selected from the ORFs having the nucleotide sequence represented by SEQ ID NO: 458.

The probes having the nucleotide sequence represented by SEQ ID NOS: 10954, 10955, 10956, 10957, 10958, 10959, 10960, 10961, 10962, 10963, 10964 were selected from the ORFs having the nucleotide sequence represented by SEQ ID NO: 459.

The probes having the nucleotide sequence represented by SEQ ID NOS: 10965, 10966, 10967, 10968, 10969, 10970, 10971, 10972, 10973, 10974, 10975 were selected from the ORFs having the nucleotide sequence represented by SEQ ID NO: 460.

The probes having the nucleotide sequence represented by SEQ ID NOS: 10976, 10977, 10978, 10979, 10980, 10981, 10982, 10983, 10984, 10985, 10986 were selected from the ORFs having the nucleotide sequence represented by SEQ ID NO: 461.

The probes having the nucleotide sequence represented by SEQ ID NOS: 10987, 10988, 10989, 10990, 10991, 10992, 10993, 10994, 10995, 10996, 10997 were selected from the ORFs having the nucleotide sequence represented by SEQ ID NO: 462.

The probes having the nucleotide sequence represented by SEQ ID NOS: 10998, 10999, 11000, 11001, 11002, 11003, 11004, 11005, 11006, 11007, 11008 were selected from the ORFs having the nucleotide sequence represented by SEQ ID NO: 463.

The probes having the nucleotide sequence represented by SEQ ID NOS: 11009, 11010, 11011, 11012, 11013, 11014, 11015, 11016, 11017, 11018, 11019 were selected from the ORFs having the nucleotide sequence represented by SEQ ID NO: 464.

The probes having the nucleotide sequence represented by SEQ ID NOS: 11020, 11021, 11022, 11023, 11024, 11025, 11026, 11027, 11028, 11029, 11030 were selected from the ORFs having the nucleotide sequence represented by SEQ ID NO: 465.

The probes having the nucleotide sequence represented by SEQ ID NOS: 11031, 11032, 11033, 11034, 11035, 11036, 11037, 11038, 11039, 11040, 11041 were selected from the ORFs having the nucleotide sequence represented by SEQ ID NO: 466.

The probes having the nucleotide sequence represented by SEQ ID NOS: 11042, 11043, 11044, 11045,.11046, 11047, 11048, 11049, 11050, 11051, 11052 were selected from the ORFs having the nucleotide sequence represented by SEQ ID NO: 467.

The probes having the nucleotide sequence represented by SEQ ID NOS: 11053, 11054, 11055, 11056, 11057, 11058, 11059, 11060, 11061, 11062, 11063 were selected from the ORFs having the nucleotide sequence represented by SEQ ID NO: 468.

The probes having the nucleotide sequence represented by SEQ ID NOS: 11064, 11065, 11066, 11067, 11068, 11069, 11070, 11071, 11072, 11073, 11074 were selected from the ORFs having the nucleotide sequence represented by SEQ ID NO: 469.

The probes having the nucleotide sequence represented by SEQ ID NOS: 11075, 11076, 11077, 11078, 11079, 11080, 11081, 11082, 11083, 11084, 11085 were selected from the ORFs having the nucleotide sequence represented by SEQ ID NO: 470.

The probes having the nucleotide sequence represented by SEQ ID NOS: 11086, 11087, 11088, 11089, 11090, 11091, 11092, 11093, 11094, 11095, 11096 were selected from the ORFs having the nucleotide sequence represented by SEQ ID NO: 471.

The probes having the nucleotide sequence represented by SEQ ID NOS: 11097, 11098, 11099, 11100, 11101, 11102, 11103, 11104, 11105, 11106, 11107 were selected from the ORFs having the nucleotide sequence represented by SEQ ID NO: 472.

The probes having the nucleotide sequence represented by SEQ ID NOS: 11108, 11109, 11110, 11111, 11112, 11113, 11114, 11115, 11116, 11117, 11118 were selected from the ORFs having the nucleotide sequence represented by SEQ ID NO: 473.

The probes having the nucleotide sequence represented by SEQ ID NOS: 11119, 11120, 11121, 11122, 11123, 11124, 11125, 11126, 11127, 11128, 11129 were selected from the ORFs having the nucleotide sequence represented by SEQ ID NO: 474.

The probes having the nucleotide sequence represented by SEQ ID NOS: 11130, 11131, 11132, 11133, 11134, 11135, 11136, 11137, 11138, 11139, 11140 were selected from the ORFs having the nucleotide sequence represented by SEQ ID NO: 475.

The probes having the nucleotide sequence represented by SEQ ID NOS: 11141, 11142, 11143, 11144, 11145, 11146, 11147, 11148, 11149, 11150, 11151 were selected from the ORFs having the nucleotide sequence represented by SEQ ID NO: 476.

The probes having the nucleotide sequence represented by SEQ ID NOS: 11152, 11153, 11154, 11155, 11156, 11157, 11158, 11159, 11160, 11161, 11162 were selected from the ORFs having the nucleotide sequence represented by SEQ ID NO: 477.

The probes having the nucleotide sequence represented by SEQ ID NOS: 11163, 11164, 11165, 11166, 11167, 11168, 11169, 11170, 11171, 11172, 11173 were selected from the ORFs having the nucleotide sequence represented by SEQ ID NO: 478.

The probes having the nucleotide sequence represented by SEQ ID NOS: 11174, 11175, 11176, 11177, 11178, 11179, 11180, 11181, 11182, 11183, 11184 were selected from the ORFs having the nucleotide sequence represented by SEQ ID NO: 479.

The probes having the nucleotide sequence represented by SEQ ID NOS: 11185, 11186, 11187, 11188, 11189, 11190, 11191, 11192, 11193, 11194, 11195 were selected from the ORFs having the nucleotide sequence represented by SEQ ID NO: 480.

The probes having the nucleotide sequence represented by SEQ ID NOS: 11196, 11197, 11198, 11199, 11200, 11201, 11202, 11203, 11204, 11205, 11206 were selected from the ORFs having the nucleotide sequence represented by SEQ ID NO: 481.

The probes having the nucleotide sequence represented by SEQ ID NOS: 11207, 11208, 11209, 11210, 11211, 11212, 11213, 11214, 11215, 11216, 11217 were selected from the ORFs having the nucleotide sequence represented by SEQ ID NO: 482.

The probes having the nucleotide sequence represented by SEQ ID NOS: 11218, 11219, 11220, 11221, 11222, 11223, 11224, 11225, 11226, 11227, 11228 were selected from the ORFs having the nucleotide sequence represented by SEQ ID NO: 483.

The probes having the nucleotide sequence represented by SEQ ID NOS: 11229, 11230, 11231, 11232, 11233, 11234, 11235, 11236, 11237, 11238, 11239 were selected from the ORFs having the nucleotide sequence represented by SEQ ID NO: 484.

The probes having the nucleotide sequence represented by SEQ ID NOS: 11240, 11241, 11242, 11243, 11244, 11245, 11246, 11247, 11248, 11249, 11250 were selected from the ORFs having the nucleotide sequence represented by SEQ ID NO: 485.

The probes having the nucleotide sequence represented by SEQ ID NOS: 11251, 11252, 11253, 11254, 11255, 11256, 11257, 11258, 11259, 11260, 11261 were selected from the ORFs having the nucleotide sequence represented by SEQ ID NO: 486.

The probes having the nucleotide sequence represented by SEQ ID NOS: 11262, 11263, 11264, 11265, 11266, 11267, 11268, 11269, 11270, 11271, 11272 were selected from the ORFs having the nucleotide sequence represented by SEQ ID NO: 487.

The probes having the nucleotide sequence represented by SEQ ID NOS: 11273, 11274, 11275, 11276, 11277, 11278, 11279, 11280, 11281, 11282, 11283 were selected from the ORFs having the nucleotide sequence represented by SEQ ID NO: 488.

The probes having the nucleotide sequence represented by SEQ ID NOS: 11284, 11285, 11286, 11287, 11288, 11289, 11290, 11291, 11292, 11293, 11294 were selected from the ORFs having the nucleotide sequence represented by SEQ ID NO: 489.

The probes having the nucleotide sequence represented by SEQ ID NOS: 11295, 11296, 11297, 11298, 11299, 11300, 11301, 11302, 11303, 11304, 11305 were selected from the ORFs having the nucleotide sequence represented by SEQ ID NO: 490.

The probes having the nucleotide sequence represented by SEQ ID NOS: 11306, 11307, 11308, 11309, 11310, 11311, 11312, 11313, 11314, 11315, 11316 were selected from the ORFs having the nucleotide sequence represented by SEQ ID NO: 491.

The probes having the nucleotide sequence represented by SEQ ID NOS: 11317, 11318, 11319, 11320, 11321, 11322, 11323, 11324, 11325, 11326, 11327 were selected from the ORFs having the nucleotide sequence represented by SEQ ID NO: 492.

The probes having the nucleotide sequence represented by SEQ ID NOS: 11328, 11329, 11330, 11331, 11332, 11333, 11334, 11335, 11336, 11337, 11338 were selected from the ORFs having the nucleotide sequence represented by SEQ ID NO: 493.

The probes having the nucleotide sequence represented by SEQ ID NOS: 11339, 11340, 11341, 11342, 11343, 11344, 11345, 11346, 11347, 11348, 11349 were selected from the ORFs having the nucleotide sequence represented by SEQ ID NO: 494.

The probes having the nucleotide sequence represented by SEQ ID NOS: 11350, 11351, 11352, 11353, 11354, 11355, 11356, 11357, 11358, 11359, 11360 were selected from the ORFs having the nucleotide sequence represented by SEQ ID NO: 495.

The probes having the nucleotide sequence represented by SEQ ID NOS: 11361, 11362, 11363, 11364, 11365, 11366, 11367, 11368, 11369, 11370, 11371 were selected from the ORFs having the nucleotide sequence represented by SEQ ID NO: 496.

The probes having the nucleotide sequence represented by SEQ ID NOS: 11372, 11373, 11374, 11375, 11376, 11377, 11378, 11379, 11380, 11381, 11382 were selected from the ORFs having the nucleotide sequence represented by SEQ ID NO: 497.

The probes having the nucleotide sequence represented by SEQ ID NOS: 11383, 11384, 11385, 11386, 11387, 11388, 11389, 11390, 11391, 11392, 11393 were selected from the ORFs having the nucleotide sequence represented by SEQ ID NO: 498.

The probes having the nucleotide sequence represented by SEQ ID NOS: 11394, 11395, 11396, 11397, 11398, 11399, 11400, 11401, 11402, 11403, 11404 were selected from the ORFs having the nucleotide sequence represented by SEQ ID NO: 499.

The probes having the nucleotide sequence represented by SEQ ID NOS: 11405, 11406, 11407, 11408, 11409, 11410, 11411, 11412, 11413, 11414, 11415 were selected from the ORFs having the nucleotide sequence represented by SEQ ID NO: 500.

The probes having the nucleotide sequence represented by SEQ ID NOS: 11416, 11417, 11418, 11419, 11420, 11421, 11422, 11423, 11424, 11425, 11426 were selected from the ORFs having the nucleotide sequence represented by SEQ ID NO: 501.

The probes having the nucleotide sequence represented by SEQ ID NOS: 11427, 11428, 11429, 11430, 11431, 11432, 11433, 11434, 11435, 11436, 11437 were selected from the ORFs having the nucleotide sequence represented by SEQ ID NO: 502.

The probes having the nucleotide sequence represented by SEQ ID NOS: 11438, 11439, 11440, 11441, 11442, 11443, 11444, 11445, 11446, 11447, 11448 were selected from the ORFs having the nucleotide sequence represented by SEQ ID NO: 503.

The probes having the nucleotide sequence represented by SEQ ID NOS: 11449, 11450, 11451, 11452, 11453, 11454, 11455, 11456, 11457, 11458, 11459 were selected from the ORFs having the nucleotide sequence represented by SEQ ID NO: 504.

The probes having the nucleotide sequence represented by SEQ ID NOS: 11460, 11461, 11462, 11463, 11464, 11465, 11466, 11467, 11468, 11469, 11470 were selected from the ORFs having the nucleotide sequence represented by SEQ ID NO: 505.

The probes having the nucleotide sequence represented by SEQ ID NOS: 11471, 11472, 11473, 11474, 11475, 11476, 11477, 11478, 11479, 11480, 11481 were selected from the ORFs having the nucleotide sequence represented by SEQ ID NO: 506.

The probes having the nucleotide sequence represented by SEQ ID NOS: 11482, 11483, 11484, 11485, 11486, 11487, 11488, 11489, 11490, 11491, 11492 were selected from the ORFs having the nucleotide sequence represented by SEQ ID NO: 507.

The probes having the nucleotide sequence represented by SEQ ID NOS: 11493, 11494, 11495, 11496, 11497, 11498, 11499, 11500, 11501, 11502, 11503 were selected from the ORFs having the nucleotide sequence represented by SEQ ID NO: 508.

The probes having the nucleotide sequence represented by SEQ ID NOS: 11504, 11505, 11506, 11507, 11508, 11509, 11510, 11511, 11512, 11513, 11514 were selected from the ORFs having the nucleotide sequence represented by SEQ ID NO: 509.

The probes having the nucleotide sequence represented by SEQ ID NOS: 11515, 11516, 11517, 11518, 11519, 11520, 11521, 11522, 11523, 11524, 11525 were selected from the ORFs having the nucleotide sequence represented by SEQ ID NO: 510.

The probes having the nucleotide sequence represented by SEQ ID NOS: 11526, 11527, 11528, 11529, 11530, 11531, 11532, 11533, 11534, 11535, 11536 were selected from the ORFs having the nucleotide sequence represented by SEQ ID NO: 511.

The probes having the nucleotide sequence represented by SEQ ID NOS: 11537, 11538, 11539, 11540, 11541, 11542, 11543, 11544, 11545, 11546, 11547 were selected from the ORFs having the nucleotide sequence represented by SEQ ID NO: 512.

The probes having the nucleotide sequence represented by SEQ ID NOS: 11548, 11549, 11550, 11551, 11552, 11553, 11554, 11555, 11556, 11557, 11558 were selected from the ORFs having the nucleotide sequence represented by SEQ ID NO: 513.

The probes having the nucleotide sequence represented by SEQ ID NOS: 11559, 11560, 11561, 11562, 11563, 11564, 11565, 11566, 11567, 11568, 11569 were selected from the ORFs having the nucleotide sequence represented by SEQ ID NO: 514.

The probes having the nucleotide sequence represented by SEQ ID NOS: 11570, 11571, 11572, 11573, 11574, 11575, 11576, 11577, 11578, 11579, 11580 were selected from the ORFs having the nucleotide sequence represented by SEQ ID NO: 515.

The probes having the nucleotide sequence represented by SEQ ID NOS: 11581, 11582, 11583, 11584, 11585, 11586, 11587, 11588, 11589, 11590, 11591 were selected from the ORFs having the nucleotide sequence represented by SEQ ID NO: 516.

The probes having the nucleotide sequence represented by SEQ ID NOS: 11592, 11593, 11594, 11595, 11596, 11597, 11598, 11599, 11600, 11601, 11602 were selected from the ORFs having the nucleotide sequence represented by SEQ ID NO: 517.

The probes having the nucleotide sequence represented by SEQ ID NOS: 11603, 11604, 11605, 11606, 11607, 11608, 11609, 11610, 11611, 11612, 11613 were selected from the ORFs having the nucleotide sequence represented by SEQ ID NO: 518.

The probes having the nucleotide sequence represented by SEQ ID NOS: 11614, 11615, 11616, 11617, 11618, 11619, 11620, 11621, 11622, 11623, 11624 were selected from the ORFs having the nucleotide sequence represented by SEQ ID NO: 519.

The probes having the nucleotide sequence represented by SEQ ID NOS: 11625, 11626, 11627, 11628, 11629, 11630, 11631, 11632, 11633, 11634, 11635 were selected from the ORFs having the nucleotide sequence represented by SEQ ID NO: 520.

The probes having the nucleotide sequence represented by SEQ ID NOS: 11636, 11637, 11638, 11639, 11640, 11641, 11642, 11643, 11644, 11645, 11646 were selected from the ORFs having the nucleotide sequence represented by SEQ ID NO: 521.

The probes having the nucleotide sequence represented by SEQ ID NOS: 11647, 11648, 11649, 11650, 11651, 11652, 11653, 11654, 11655, 11656, 11657 were selected from the ORFs having the nucleotide sequence represented by SEQ ID NO: 522.

The probes having the nucleotide sequence represented by SEQ ID NOS: 11658, 11659, 11660, 11661, 11662, 11663, 11664, 11665, 11666, 11667, 11668 were selected from the ORFs having the nucleotide sequence represented by SEQ ID NO: 523.

The probes having the nucleotide sequence represented by SEQ ID NOS: 11669, 11670, 11671, 11672, 11673, 11674, 11675, 11676, 11677, 11678, 11679 were selected from the ORFs having the nucleotide sequence represented by SEQ ID NO: 524.

The probes having the nucleotide sequence represented by SEQ ID NOS: 11680, 11681, 11682, 11683, 11684, 11685, 11686, 11687, 11688, 11689, 11690 were selected from the ORFs having the nucleotide sequence represented by SEQ ID NO: 525.

The probes having the nucleotide sequence represented by SEQ ID NOS: 11691, 11692, 11693, 11694, 11695, 11696, 11697, 11698, 11699, 11700, 11701 were selected from the ORFs having the nucleotide sequence represented by SEQ ID NO: 526.

The probes having the nucleotide sequence represented by SEQ ID NOS: 11702, 11703, 11704, 11705, 11706, 11707, 11708, 11709, 11710, 11711, 11712 were selected from the ORFs having the nucleotide sequence represented by SEQ ID NO: 527.

The probes having the nucleotide sequence represented by SEQ ID NOS: 11713, 11714, 11715, 11716, 11717, 11718, 11719, 11720, 11721, 11722, 11723 were selected from the ORFs having the nucleotide sequence represented by SEQ ID NO: 528.

The probes having the nucleotide sequence represented by SEQ ID NOS: 11724, 11725, 11726, 11727, 11728, 11729, 11730, 11731, 11732, 11733, 11734 were selected from the ORFs having the nucleotide sequence represented by SEQ ID NO: 529.

The probes having the nucleotide sequence represented by SEQ ID NOS: 11735, 11736, 11737, 11738, 11739, 11740, 11741, 11742, 11743, 11744, 11745 were selected from the ORFs having the nucleotide sequence represented by SEQ ID NO: 530.

The probes having the nucleotide sequence represented by SEQ ID NOS: 11746, 11747, 11748, 11749, 11750, 11751, 11752, 11753, 11754, 11755, 11756 were selected from the ORFs having the nucleotide sequence represented by SEQ ID NO: 531.

The probes having the nucleotide sequence represented by SEQ ID NOS: 11757, 11758, 11759, 11760, 11761, 11762, 11763, 11764, 11765, 11766, 11767 were selected from the ORFs having the nucleotide sequence represented by SEQ ID NO: 532.

The probes having the nucleotide sequence represented by SEQ ID NOS: 11768, 11769, 11770, 11771, 11772, 11773, 11774, 11775, 11776, 11777, 11778 were selected from the ORFs having the nucleotide sequence represented by SEQ ID NO: 533.

The probes having the nucleotide sequence represented by SEQ ID NOS: 11779, 11780, 11781, 11782, 11783, 11784, 11785, 11786, 11787, 11788, 11789 were selected from the ORFs having the nucleotide sequence represented by SEQ ID NO: 534.

The probes having the nucleotide sequence represented by SEQ ID NOS: 11790, 11791, 11792, 11793, 11794, 11795, 11796, 11797, 11798, 11799, 11800 were selected from the ORFs having the nucleotide sequence represented by SEQ ID NO: 535.

The probes having the nucleotide sequence represented by SEQ ID NOS: 11801, 11802, 11803, 11804, 11805, 11806, 11807, 11808, 11809, 11810, 11811 were selected from the ORFs having the nucleotide sequence represented by SEQ ID NO: 536.

The probes having the nucleotide sequence represented by SEQ ID NOS: 11812, 11813, 11814, 11815, 11816, 11817, 11818, 11819, 11820, 11821, 11822 were selected from the ORFs having the nucleotide sequence represented by SEQ ID NO: 537.

The probes having the nucleotide sequence represented by SEQ ID NOS: 11823, 11824, 11825, 11826, 11827, 11828, 11829, 11830, 11831, 11832, 11833 were selected from the ORFs having the nucleotide sequence represented by SEQ ID NO: 538.

The probes having the nucleotide sequence represented by SEQ ID NOS: 11834, 11835, 11836, 11837, 11838, 11839, 11840, 11841, 11842, 11843, 11844 were selected from the ORFs having the nucleotide sequence represented by SEQ ID NO: 539.

The probes having the nucleotide sequence represented by SEQ ID NOS: 11845, 11846, 11847, 11848, 11849, 11850, 11851, 11852, 11853, 11854, 11855 were selected from the ORFs having the nucleotide sequence represented by SEQ ID NO: 540.

The probes having the nucleotide sequence represented by SEQ ID NOS: 11856, 11857, 11858, 11859, 11860, 11861, 11862, 11863, 11864, 11865, 11866 were selected from the ORFs having the nucleotide sequence represented by SEQ ID NO: 541.

The probes having the nucleotide sequence represented by SEQ ID NOS: 11867, 11868, 11869, 11870, 11871, 11872, 11873, 11874, 11875, 11876, 11877 were selected from the ORFs having the nucleotide sequence represented by SEQ ID NO: 542.

The probes having the nucleotide sequence represented by SEQ ID NOS: 11878, 11879, 11880, 11881, 11882, 11883, 11884, 11885, 11886, 11887, 11888 were selected from the. ORFs having the nucleotide sequence represented by SEQ ID NO: 543.

The probes having the nucleotide sequence represented by SEQ ID NOS: 11889, 11890, 11891, 11892, 11893, 11894, 11895, 11896, 11897, 11898, 11899 were selected from the ORFs having the nucleotide sequence represented by SEQ ID NO: 544.

The probes having the nucleotide sequence represented by SEQ ID NOS: 11900, 11901, 11902, 11903, 11904, 11905, 11906, 11907, 11908, 11909, 11910 were selected from the ORFs having the nucleotide sequence represented by SEQ ID NO: 545.

The probes having the nucleotide sequence represented by SEQ ID NOS: 11911, 11912, 11913, 11914, 11915, 11916, 11917, 11918, 11919, 11920, 11921 were selected from the ORFs having the nucleotide sequence represented by SEQ ID NO: 546.

The probes having the nucleotide sequence represented by SEQ ID NOS: 11922, 11923, 11924, 11925, 11926, 11927, 11928, 11929, 11930, 11931, 11932 were selected from the ORFs having the nucleotide sequence represented by SEQ ID NO: 547.

The probes having the nucleotide sequence represented by SEQ ID NOS: 11933, 11934, 11935, 11936, 11937, 11938, 11939, 11940, 11941, 11942, 11943 were selected from the ORFs having the nucleotide sequence represented by SEQ ID NO: 548.

The probes having the nucleotide sequence represented by SEQ ID NOS: 11944, 11945, 1194.6, 11947, 11948, 11949, 11950, 11951, 11952, 11953, 11954 were selected from the ORFs having the nucleotide sequence represented by SEQ ID NO: 549.

The probes having the nucleotide sequence represented by SEQ ID NOS: 11955, 11956, 11957, 11958, 11959, 11960, 11961, 11962, 11963, 11964, 11965 were selected from the ORFs having the nucleotide sequence represented by SEQ ID NO: 550.

The probes having the nucleotide sequence represented by SEQ ID NOS: 11966, 11967, 11968, 11969, 11970, 11971, 11972, 11973, 11974, 11975, 11976 were selected from the ORFs having the nuclebtide sequence represented by SEQ ID NO: 551.

The probes having the nucleotide sequence represented by SEQ ID NOS: 11977, 11978, 11979, 11980, 11981, 11982, 11983, 11984, 11985, 11986, 11987 were selected from the ORFs having the nucleotide sequence represented by SEQ ID NO: 552.

The probes having the nucleotide sequence represented by SEQ ID NOS: 11988, 11989, 11990, 11991, 11992, 11993, 11994, 11995, 11996, 11997, 11998 were selected from the ORFs having the nucleotide sequence represented by SEQ ID NO: 553.

The probes having the nucleotide sequence represented by SEQ ID NOS: 11999, 12000, 12001, 12002, 12003, 12004, 12005, 12006, 12007, 12008, 12009 were selected from the ORFs having the nucleotide sequence represented by SEQ ID NO: 554.

The probes having the nucleotide sequence represented by SEQ ID NOS: 12010, 12011, 12012, 12013, 12014, 12015, 12016, 12017, 12018, 12019, 12020 were selected from the ORFs having the nucleotide sequence represented by SEQ ID NO: 555.

The probes having the nucleotide sequence represented by SEQ ID NOS: 12021, 12022, 12023, 12024, 12025, 12026, 12027, 12028, 12029, 12030, 12031 were selected from the ORFs having the nucleotide sequence represented by SEQ ID NO: 556.

The probes having the nucleotide sequence represented by SEQ ID NOS: 12032, 12033, 12034, 12035, 12036, 12037, 12038, 12039, 12040, 12041, 12042 were selected from the ORFs having the nucleotide sequence represented by SEQ ID NO: 557.

The probes having the nucleotide sequence represented by SEQ ID NOS: 12043, 12044, 12045, 12046, 12047, 12048, 12049, 12050, 12051, 12052, 12053 were selected from the ORFs having the nucleotide sequence represented by SEQ ID NO: 558.

The probes having the nucleotide sequence represented by SEQ ID NOS: 12054, 12055, 12056, 12057, 12058, 12059, 12060, 12061, 12062, 12063, 12064 were selected from the ORFs having the nucleotide sequence represented by SEQ ID NO: 559.

The probes having the nucleotide sequence represented by SEQ ID NOS: 12065, 12066, 12067, 12068, 12069, 12070, 12071, 12072, 12073, 12074, 12075 were selected from the ORFs having the nucleotide sequence represented by SEQ ID NO: 560.

The probes having the nucleotide sequence represented by SEQ ID NOS: 12076, 12077, 12078, 12079, 12080, 12081, 12082, 12083, 12084, 12085, 12086 were selected from the ORFs having the nucleotide sequence represented by SEQ ID NO: 561.

The probes having the nucleotide sequence represented by SEQ ID NOS: 12087, 12088, 12089, 12090, 12091, 12092, 12093, 12094, 12095, 12096, 12097 were selected from the ORFs having the nucleotide sequence represented by SEQ ID NO: 562.

The probes having the nucleotide sequence represented by SEQ ID NOS: 12098, 12099, 12100, 12101, 12102, 12103, 12104, 12105, 12106, 12107, 12108 were selected from the ORFs having the nucleotide sequence represented by SEQ ID NO: 563.

The probes having the nucleotide sequence represented by SEQ ID NOS: 12109, 12110, 12111, 12112, 12113, 12114, 12115, 12116, 12117, 12118, 12119 were selected from the ORFs having the nucleotide sequence represented by SEQ ID NO: 564.

The probes having the nucleotide sequence represented by SEQ ID NOS: 12120, 12121,12122, 12123, 12124, 12125, 12126, 12127, 12128, 12129, 12130 were selected from the ORFs having the nucleotide sequence represented by SEQ ID NO: 565.

The probes having the nucleotide sequence represented by SEQ ID NOS: 12131, 12132, 12133, 12134, 12135, 12136, 12137, 12138, 12139, 12140, 12141 were selected from the ORFs having the nucleotide sequence represented by SEQ ID NO: 566.

The probes having the nucleotide sequence represented by SEQ ID NOS: 12142, 12143, 12144, 12145, 12146, 12147, 12148, 12149, 12150, 12151, 12152 were selected from the ORFs having the nucleotide sequence represented by SEQ ID NO: 567.

The probes having the nucleotide sequence represented by SEQ ID NOS: 12153, 12154, 12155, 12156, 12157, 12158, 12159, 12160, 12161, 12162, 12163 were selected from the ORFs having the nucleotide sequence represented by SEQ ID NO: 568.

The probes having the nucleotide sequence represented by SEQ ID NOS: 12164, 12165, 12166, 12167, 12168, 12169, 12170, 12171, 12172, 12173, 12174 were selected from the ORFs having the nucleotide sequence represented by SEQ ID NO: 569.

The probes having the nucleotide sequence represented by SEQ ID NOS: 12175, 12176, 12177, 12178, 12179, 12180, 12181, 12182, 12183, 12184, 12185 were selected from the ORFs having the nucleotide sequence represented by SEQ ID NO: 570.

The probes having the nucleotide sequence represented by SEQ ID NOS: 12186, 12187, 12188, 12189, 12190, 12191, 12192, 12193, 12194, 12195, 12196 were selected from the ORFs having the nucleotide sequence represented by SEQ ID NO: 571.

The probes having the nucleotide sequence represented by SEQ ID NOS: 12197, 12198, 12199, 12200, 12201, 12202, 12203, 12204, 12205, 12206, 12207 were selected from the ORFs having the nucleotide sequence represented by SEQ ID NO: 572.

The probes having the nucleotide sequence represented by SEQ ID NOS: 12208, 12209, 12210, 12211, 12212, 12213, 12214, 12215, 12216, 12217, 12218 were selected from the ORFs having the nucleotide sequence represented by SEQ ID NO: 573.

The probes having the nucleotide sequence represented by SEQ ID NOS: 12219, 12220, 12221, 12222, 12223, 12224, 12225, 12226, 12227, 12228, 12229 were selected from the ORFs having the nucleotide sequence represented by SEQ ID NO: 574.

The probes having the nucleotide sequence represented by SEQ ID NOS: 12230, 12231, 12232, 12233, 12234, 12235, 12236, 12237, 12238, 12239, 12240 were selected from the ORFs having the nucleotide sequence represented by SEQ ID NO: 575.

The probes having the nucleotide sequence represented by SEQ ID NOS: 12241, 12242, 12243, 12244, 12245, 12246, 12247, 12248, 12249, 12250, 12251 were selected from the ORFs having the nucleotide sequence represented by SEQ ID NO: 576.

The probes having the nucleotide sequence represented by SEQ ID NOS: 12252, 12253, 12254, 12255, 12256, 12257, 12258, 12259, 12260, 12261, 12262 were selected from the ORFs having the nucleotide sequence represented by SEQ ID NO: 577.

The probes having the nucleotide sequence represented by SEQ ID NOS: 12263, 12264, 12265, 12266, 12267, 12268, 12269, 12270, 12271, 12272, 12273 were selected from the ORFs having the nucleotide sequence represented by SEQ ID NO: 578.

The probes having the nucleotide sequence represented by SEQ ID NOS: 12274, 12275, 12276, 12277, 12278, 12279, 12280, 12281, 12282, 12283, 12284 were selected from the ORFs having the nucleotide sequence represented by SEQ ID NO: 579.

The probes having the nucleotide sequence represented by SEQ ID NOS: 12285, 12286, 12287, 12288, 12289, 12290, 12291, 12292, 12293, 12294, 12295 were selected from the ORFs having the nucleotide sequence represented by SEQ ID NO: 580.

The probes having the nucleotide sequence represented by SEQ ID NOS: 12296, 12297, 12298, 12299, 12300, 12301, 12302, 12303, 12304, 12305, 12306 were selected from the ORFs having the nucleotide sequence represented by SEQ ID NO: 581.

The probes having the nucleotide sequence represented by SEQ ID NOS: 12307, 12308, 12309, 12310, 12311, 12312, 12313, 12314, 12315, 12316, 12317 were selected from the ORFs having the nucleotide sequence represented by SEQ ID NO: 582.

The probes having the nucleotide sequence represented by SEQ ID NOS: 12318, 12319, 12320, 12321, 12322, 12323, 12324, 12325, 12326, 12327, 12328 were selected from the ORFs having the nucleotide sequence represented by SEQ ID NO: 583.

The probes having the nucleotide sequence represented by SEQ ID NOS: 12329, 12330, 12331, 12332, 12333, 12334, 12335, 12336, 12337, 12338, 12339 were selected from the ORFs having the nucleotide sequence represented by SEQ ID NO: 584.

The probes having the nucleotide sequence represented by SEQ ID NOS: 12340, 12341, 12342, 12343, 12344, 12345, 12346, 12347, 12348, 12349, 12350 were selected from the ORFs having the nucleotide sequence represented by SEQ ID NO: 585.

The probes having the nucleotide sequence represented by SEQ ID NOS: 12351, 12352, 12353, 12354, 12355, 12356, 12357, 12358, 12359, 12360, 12361 were selected from the ORFs having the nucleotide sequence represented by SEQ ID NO: 586.

The probes having the nucleotide sequence represented by SEQ ID NOS: 12362, 12363, 12364, 12365, 12366, 12367, 12368, 12369, 12370, 12371, 12372 were selected from the ORFs having the nucleotide sequence represented by SEQ ID NO: 587.

The probes having the nucleotide sequence represented by SEQ ID NOS: 12373, 12374, 12375, 12376, 12377, 12378, 12379, 12380, 12381, 12382, 12383 were selected from the ORFs having the nucleotide sequence represented by SEQ ID NO: 588.

The probes having the nucleotide sequence represented by SEQ ID NOS: 12384, 12385, 12386, 12387, 12388, 12389, 12390, 12391, 12392, 12393, 12394 were selected from the ORFs having the nucleotide sequence represented by SEQ ID NO: 589.

The probes having the nucleotide sequence represented by SEQ ID NOS: 12395, 12396, 12397, 12398, 12399, 12400, 12401, 12402, 12403, 12404, 12405 were selected from the ORFs having the nucleotide sequence represented by SEQ ID NO: 590.

The probes having the nucleotide sequence represented by SEQ ID NOS: 12406, 12407, 12408, 12409, 12410, 12411, 12412, 12413, 12414, 12415, 12416 were selected from the ORFs having the nucleotide sequence represented by SEQ ID NO: 591.

The probes having the nucleotide sequence represented by SEQ ID NOS: 12417., 12418, 12419, 12420, 12421, 12422, 12423, 12424, 12425, 12426, 12427 were selected from the ORFs having the nucleotide sequence represented by SEQ ID NO: 592.

The probes having the nucleotide sequence represented by SEQ ID NOS: 12428, 12429, 12430, 12431, 12432, 12433, 12434, 12435, 12436, 12437, 12438 were selected from the ORFs having the nucleotide sequence represented by SEQ ID NO: 593.

The probes having the nucleotide sequence represented by SEQ ID NOS: 12439, 12440, 12441, 12442, 12443, 12444, 12445, 12446, 12447, 12448, 12449 were selected from the ORFs having the nucleotide sequence represented by SEQ ID NO: 594.

The probes having the nucleotide sequence represented by SEQ ID NOS: 12450, 12451, 12452, 12453, 12454, 12455, 12456, 12457, 12458, 12459, 12460 were selected from the ORFs having the nucleotide sequence represented by SEQ ID NO: 595.

The probes having the nucleotide sequence represented by SEQ ID NOS: 12461, 12462, 12463, 12464, 12465, 12466, 12467, 12468, 12469, 12470, 12471 were selected from the ORFs having the nucleotide sequence represented by SEQ ID NO: 596.

The probes having the nucleotide sequence represented by SEQ ID NOS: 12472, 12473, 12474, 12475, 12476, 12477, 12478, 12479, 12480, 12481, 12482 were selected from the ORFs having the nucleotide sequence represented by SEQ ID NO: 597.

The probes having the nucleotide sequence represented by SEQ ID NOS: 12483, 12484, 12485, 12486, 12487, 12488, 12489, 12490, 12491, 12492, 12493 were selected from the ORFs having the nucleotide sequence represented by SEQ ID NO: 598.

The probes having the nucleotide sequence represented by SEQ ID NOS: 12494, 12495, 12496, 12497, 12498, 12499, 12500, 12501, 12502, 12503, 12504 were selected from the ORFs having the nucleotide sequence represented by SEQ ID NO: 599.

The probes having the nucleotide sequence represented by SEQ ID NOS: 12505, 12506, 12507, 12508, 12509, 12510, 12511, 12512, 12513, 12514, 12515 were selected from the ORFs having the nucleotide sequence represented by SEQ ID NO: 600.

The probes having the nucleotide sequence represented by SEQ ID NOS: 12516, 12517, 12518, 12519, 12520, 12521, 12522, 12523, 12524, 12525, 12526 were selected from the ORFs having the nucleotide sequence represented by SEQ ID NO: 601.

The probes having the nucleotide sequence represented by SEQ ID NOS: 12527, 12528, 12529, 12530, 12531, 12532, 12533, 12534, 12535, 12536, 12537 were selected from the ORFs having the nucleotide sequence represented by SEQ ID NO: 602.

The probes having the nucleotide sequence represented by SEQ ID NOS: 12538, 12539, 12540, 12541, 12542, 12543, 12544, 12545, 12546, 12547, 12548 were selected from the ORFs having the nucleotide sequence represented by SEQ ID NO: 603.

The probes having the nucleotide sequence represented by SEQ ID NOS: 12549, 12550, 12551, 12552, 12553, 12554, 12555, 12556, 12557, 12558, 12559 were selected from the ORFs having the nucleotide sequence represented by SEQ ID NO: 604.

The probes having the nucleotide sequence represented by SEQ ID NOS: 12560, 12561, 12562, 12563, 12564, 12565, 12566, 12567, 12568, 12569, 12570 were selected from the ORFs having the nucleotide sequence represented by SEQ ID NO: 605.

The probes having the nucleotide sequence represented by SEQ ID NOS: 12571, 12572, 12573, 12574, 12575, 12576, 12577, 12578, 12579, 12580, 12581 were selected from the ORFs having the nucleotide sequence represented by SEQ ID NO: 606.

The probes having the nucleotide sequence represented by SEQ ID NOS: 12582, 12583, 12584, 12585, 12586, 12587, 12588, 12589, 12590, 12591, 12592 were selected from the ORFs having the nucleotide sequence represented by SEQ ID NO: 607.

The probes having the nucleotide sequence represented by SEQ ID NOS: 12593, 12594, 12595, 12596, 12597, 12598, 12599, 12600, 12601, 12602, 12603 were selected from the ORFs having the nucleotide sequence represented by SEQ ID NO: 608.

The probes having the nucleotide sequence represented by SEQ ID NOS: 12604, 12605, 12606, 12607, 12608, 12609, 12610, 12611, 12612, 12613, 12614 were selected from the ORFs having the nucleotide sequence represented by SEQ ID NO: 609.

The probes having the nucleotide sequence represented by SEQ ID NOS: 12615, 12616, 12617, 12618, 12619, 12620, 12621, 12622, 12623, 12624, 12625 were selected from the ORFs having the nucleotide sequence represented by SEQ ID NO: 610.

The probes having the nucleotide sequence represented by. SEQ ID NOS: 12626, 12627, 12628, 12629, 12630, 12631, 12632, 12633, 12634, 12635, 12636 were selected from the ORFs having the nucleotide sequence represented by SEQ ID NO: 611.

The probes having the nucleotide sequence represented by SEQ ID NOS: 12637, 12638, 12639, 12640, 12641, 12642, 12643, 12644, 12645, 12646, 12647 were selected from the ORFs having the nucleotide sequence represented by SEQ ID NO: 612.

The probes having the nucleotide sequence represented by SEQ ID NOS: 12648, 12649, 12650, 12651, 12652, 12653, 12654, 12655, 12656, 12657, 12658 were selected from the ORFs having the nucleotide sequence represented by SEQ ID NO: 613.

The probes having the nucleotide sequence represented by SEQ ID NOS: 12659, 12660, 12661, 12662, 12663, 12664, 12665, 12666, 12667, 12668, 12669 were selected from the ORFs having the nucleotide sequence represented by SEQ ID NO: 614.

The probes having the nucleotide sequence represented by SEQ ID NOS: 12670, 12671, 12672, 12673, 12674, 12675, 12676, 12677, 12678, 12679, 12680 were selected from the ORFs having the nucleotide sequence represented by SEQ ID NO: 615.

The probes having the nucleotide sequence represented by SEQ ID NOS: 12681, 12682, 12683, 12684, 12685, 12686, 12687, 12688, 12689, 12690, 12691 were selected from the ORFs having the nucleotide sequence represented by SEQ ID NO: 616.

The probes having the nucleotide sequence represented by SEQ ID NOS: 12692, 12693, 12694, 12695, 12696, 12697, 12698, 12699, 12700, 12701, 12702 were selected from the ORFs having the nucleotide sequence represented by SEQ ID NO: 617.

The probes having the nucleotide sequence represented by SEQ ID NOS: 12703, 12704, 12705, 12706, 12707, 12708, 12709, 12710, 12711, 12712, 12713 were selected from the ORFs having the nucleotide sequence represented by SEQ ID NO: 618.

The probes having the nucleotide sequence represented by SEQ ID NOS: 12714, 12715, 12716, 12717, 12718, 12719, 12720, 12721, 12722, 12723, 12724 were selected from the ORFs having the nucleotide sequence represented by SEQ ID NO: 619.

The probes having the nucleotide sequence represented by SEQ ID NOS: 12725, 12726, 12727, 12728, 12729, 12730, 12731, 12732, 12733, 12734, 12735 were selected from the ORFs having the nucleotide sequence represented by SEQ ID NO: 620.

The probes having the nucleotide sequence represented by SEQ ID NOS: 12736, 12737, 12738, 12739, 12740, 12741, 12742, 12743, 12744, 12745, 12746 were selected from the ORFs having the nucleotide sequence represented by SEQ ID NO: 621.

The probes having the nucleotide sequence represented by SEQ ID NOS: 12747, 12748, 12749, 12750, 12751, 12752, 12753, 12754, 12755, 12756, 12757 were selected-from the ORFs having the nucleotide sequence represented by SEQ ID NO: 622.

The probes having the nucleotide sequence represented by SEQ ID NOS: 12758, 12759, 12760, 12761, 12762, 12763, 12764, 12765, 12766, 12767, 12768 were selected from the ORFs having the nucleotide sequence represented by SEQ ID NO: 623.

The probes having the nucleotide sequence represented by SEQ ID NOS: 12769, 12770, 12771, 12772, 12773, 12774, 12775, 12776, 12777, 12778, 12779 were selected from the ORFs having the nucleotide sequence represented by SEQ ID NO: 624.

The probes having the nucleotide sequence represented by SEQ ID NOS: 12780, 12781, 12782, 12783, 12784, 12785, 12786, 12787, 12788, 12789, 12790 were selected from the ORFs having the nucleotide sequence represented by SEQ ID NO: 625.

The probes having the nucleotide sequence represented by SEQ ID NOS: 12791, 12792, 12793, 12794, 12795, 12796, 12797, 12798, 12799, 12800, 12801 were selected from the ORFs having the nucleotide sequence represented by SEQ ID NO: 626.

The probes having the nucleotide sequence represented by SEQ ID NOS: 12802, 12803, 12804, 12805, 12806, 12807, 12808, 12809, 12810, 12811, 12812 were selected from the ORFs having the nucleotide sequence represented by SEQ ID NO: 627.

The probes having the nucleotide sequence represented by SEQ ID NOS: 12813, 12814, 12815, 12816, 12817, 12818, 12819, 12820, 12821, 12822, 12823 were selected from the ORFs having the nucleotide sequence represented by SEQ ID NO: 628.

The probes having the nucleotide sequence represented by SEQ ID NOS: 12824, 12825, 12826, 12827, 12828, 12829, 12830, 12831, 12832, 12833, 12834 were selected from the ORFs having the nucleotide sequence represented by SEQ ID NO: 629.

The probes having the nucleotide sequence represented by SEQ ID NOS: 12835, 12836, 12837, 12838, 12839, 12840, 12841, 12842, 12843, 12844, 12845 were selected from the ORFs having the nucleotide sequence represented by SEQ ID NO: 630.

The probes having the nucleotide sequence represented by SEQ ID NOS: 12846, 12847, 12848, 12849, 12850, 12851, 12852, 12853, 12854, 12855, 12856 were selected from the ORFs having the nucleotide sequence represented by SEQ ID NO: 631.

The probes having the nucleotide sequence represented by SEQ ID NOS: 12857, 12858, 12859, 12860, 12861, 12862, 12863, 12864, 12865, 12866, 12867 were selected from the ORFs having the nucleotide sequence represented by SEQ ID NO: 632.

The probes having the nucleotide sequence represented by SEQ ID NOS: 12868, 12869, 12870, 12871, 12872, 12873, 12874, 12875, 12876, 12877, 12878 were selected from the ORFs having the nucleotide sequence represented by SEQ ID NO: 633.

The probes having the nucleotide sequence represented by SEQ ID NOS: 12879, 12880, 12881, 12882, 12883, 12884, 12885, 12886, 12887, 12888, 12889 were selected from the ORFs having the nucleotide sequence represented by SEQ ID NO: 634.

The probes having the nucleotide sequence represented by SEQ ID NOS: 12890, 12891, 12892, 12893, 12894, 12895, 12896, 12897, 12898, 12899, 12900 were selected from the ORFs having the nucleotide sequence represented by SEQ ID NO: 635.

The probes having the nucleotide sequence represented by SEQ ID NOS: 12901, 12902, 12903, 12904, 12905, 12906, 12907, 12908, 12909, 12910, 12911 were selected from the ORFs having the nucleotide sequence represented by SEQ ID NO: 636.

The probes having the nucleotide sequence represented by SEQ ID NOS: 12912, 12913, 12914, 12915, 12916, 12917, 12918, 12919, 12920, 12921, 12922 were selected from the ORFs having the nucleotide sequence represented by SEQ ID NO: 637.

The probes having the nucleotide sequence represented by SEQ ID NOS: 12923, 12924, 12925, 12926, 12927, 12928, 12929, 12930, 12931, 12932, 12933 were selected from the ORFs having the nucleotide sequence represented by SEQ ID NO: 638.

The probes having the nucleotide sequence represented by SEQ ID NOS: 12934, 12935, 12936, 12937, 12938, 12939, 12940, 12941, 12942, 12943, 12944 were selected from the ORFs having the nucleotide sequence represented by SEQ ID NO: 639.

The probes having the nucleotide sequence represented by SEQ ID NOS: 12945, 12946, 12947, 12948, 12949, 12950, 12951, 12952, 12953, 12954, 12955 were selected from the ORFs having the nucleotide sequence represented by SEQ ID NO: 640.

The probes having the nucleotide sequence represented by SEQ ID NOS: 12956, 12957, 12958, 12959, 12960, 12961, 12962, 12963, 12964, 12965, 12966 were selected from the ORFs having the nucleotide sequence represented by SEQ ID NO: 641.

The probes having the nucleotide sequence represented by SEQ ID NOS: 12967, 12968, 12969, 12970, 12971, 12972, 12973, 12974, 12975, 12976, 12977 were selected from the ORFs having the nucleotide sequence represented by SEQ ID NO: 642.

The probes having the nucleotide sequence represented by SEQ ID NOS: 12978, 12979, 12980, 12981, 12982, 12983, 12984, 12985, 12986, 12987, 12988 were selected from the ORFs having the nucleotide sequence represented by SEQ ID NO: 643.

The probes having the nucleotide sequence represented by SEQ ID NOS: 12989, 12990, 12991, 12992, 12993, 12994, 12995, 12996, 12997, 12998, 12999 were selected from the ORFs having the nucleotide sequence represented by SEQ ID NO: 644.

The probes having the nucleotide sequence represented by SEQ ID NOS: 13000, 13001, 13002, 13003, 13004, 13005, 13006, 13007, 13008, 13009, 13010 were selected from the ORFs having the nucleotide sequence represented by SEQ ID NO: 645.

The probes having the nucleotide sequence represented by SEQ ID NOS: 13011, 13012, 13013, 13014, 13015, 13016, 13017, 13018, 13019, 13020, 13021 were selected from the ORFs having the nucleotide sequence represented by SEQ ID NO: 646.

The probes having the nucleotide sequence represented by SEQ ID NOS: 13022, 13023, 13024, 13025, 13026, 13027, 13028, 13029, 13030, 13031, 13032 were selected from the ORFs having the nucleotide sequence represented by SEQ ID NO: 647.

The probes having the nucleotide sequence represented by SEQ ID NOS: 13033, 13034, 13035, 13036, 13037, 13038, 13039, 13040, 13041, 13042, 13043 were selected from the ORFs having the nucleotide sequence represented by SEQ ID NO: 648.

The probes having the nucleotide sequence represented by SEQ ID NOS: 13044, 13045, 13046, 13047, 13048, 13049, 13050, 13051, 13052, 13053, 13054 were selected from the ORFs having the nucleotide sequence represented by SEQ ID NO: 649.

The probes having the nucleotide sequence represented by SEQ ID NOS: 13055, 13056, 13057, 13058, 13059, 13060, 13061, 13062, 13063, 13064, 13065 were selected from the ORFs having the nucleotide sequence represented by SEQ ID NO: 650.

The probes having the nucleotide sequence represented by SEQ ID NOS: 13066, 13067, 13068, 13069, 13070, 13071, 13072, 13073, 13074, 13075, 13076 were selected from the ORFs having the nucleotide sequence represented by SEQ ID NO: 651.

The probes having the nucleotide sequence represented by SEQ ID NOS: 13077, 13078, 13079, 13080, 13081, 13082, 13083, 13084, 13085, 13086, 13087 were selected from the ORFs having the nucleotide sequence represented by SEQ ID NO: 652.

The probes having the nucleotide sequence represented by SEQ ID NOS: 13088, 13089, 13090, 13091, 13092, 13093, 13094, 13095, 13096, 13097, 13098 were selected from the ORFs having the nucleotide sequence represented by SEQ ID NO: 653.

The probes having the nucleotide sequence represented by SEQ ID NOS: 13099, 13100, 13101, 13102, 13103, 13104, 13105, 13106, 13107, 13108, 13109 were selected from the ORFs having the nucleotide sequence represented by SEQ ID NO: 654.

The probes having the nucleotide sequence represented by SEQ ID NOS: 13110, 13111, 13112, 13113, 13114, 13115, 13116, 13117, 13118, 13119, 13120 were selected from the ORFs having the nucleotide sequence represented by SEQ ID NO: 655.

The probes having the nucleotide sequence represented by SEQ ID NOS: 13121, 13122, 13123, 13124, 13125, 13126, 13127, 13128, 13129, 13130, 13131 were selected from the ORFs having the nucleotide sequence represented by SEQ ID NO: 656.

The probes having the nucleotide sequence represented by SEQ ID NOS: 13132, 13133, 13134, 13135, 13136, 13137, 13138, 13139, 13140, 13141, 13142 were selected from the ORFs having the nucleotide sequence represented by SEQ ID NO: 657.

The probes having the nucleotide sequence represented by SEQ ID NOS: 13143, 13144, 13145, 13146, 13147, 13148, 13149, 13150, 13151, 13152, 13153 were selected from the ORFs having the nucleotide sequence represented by SEQ ID NO: 658.

The probes having the nucleotide sequence represented by SEQ ID NOS: 13154, 13155, 13156, 13157, 13158, 13159, 13160, 13161, 13162, 13163, 13164 were selected from the ORFs having the nucleotide sequence represented by SEQ ID NO: 659.

The probes having the nucleotide sequence represented by SEQ ID NOS: 13165, 13166, 13167, 13168, 13169, 13170, 13171, 13172, 13173, 13174, 13175 were selected from the ORFs having the nucleotide sequence represented by SEQ ID NO: 660.

The probes having the nucleotide sequence represented by SEQ ID NOS: 13176, 13177, 13178, 13179, 13180, 13181, 13182, 13183, 13184, 13185, 13186 were selected from the ORFs having the nucleotide sequence represented by SEQ ID NO: 661.

The probes having the nucleotide sequence represented by SEQ ID NOS: 13187, 13188, 13189, 13190, 13191, 13192, 13193, 13194, 13195, 13196, 13197 were selected from the ORFs having the nucleotide sequence represented by SEQ ID NO: 662.

The probes having the nucleotide sequence represented by SEQ ID NOS: 13198, 13199, 13200, 13201, 13202, 13203, 13204, 13205, 13206, 13207, 13208 were selected from the ORFs having the nucleotide sequence represented by SEQ ID NO: 663.

The probes having the nucleotide sequence represented by SEQ ID NOS: 13209, 13210, 13211, 13212, 13213, 13214, 13215, 13216, 13217, 13218, 13219 were selected from the ORFs having the nucleotide sequence represented by SEQ ID NO: 664.

The probes having the nucleotide sequence represented by SEQ ID NOS: 13220, 13221, 13222, 13223, 13224, 13225, 13226, 13227, 13228, 13229, 13230 were selected from the ORFs having the nucleotide sequence represented by SEQ ID NO: 665.

The probes having the nucleotide sequence represented by SEQ ID NOS: 13231, 13232, 13233, 13234, 13235, 13236, 13237, 13238, 13239, 13240, 13241 were selected from the ORFs having the nucleotide sequence represented by SEQ ID NO: 666.

The probes having the nucleotide sequence represented by SEQ ID NOS: 13242, 13243, 13244, 13245, 13246, 13247, 13248, 13249, 13250, 13251, 13252 were selected from the ORFs having the nucleotide sequence represented by SEQ ID NO: 667.

The probes having the nucleotide sequence represented by SEQ ID NOS: 13253, 13254, 13255, 13256, 13257, 13258, 13259, 13260, 13261, 13262, 13263 were selected from the ORFs having the nucleotide sequence represented by SEQ ID NO: 668.

The probes having the nucleotide sequence represented by SEQ ID NOS: 13264, 13265, 13266, 13267, 13268, 13269, 13270, 13271, 13272, 13273,13274 were selected from the ORFs having the nucleotide sequence represented by SEQ ID NO: 669.

The probes having the nucleotide sequence represented by SEQ ID NOS: 13275, 13276, 13277, 13278, 13279, 13280, 13281, 13282, 13283, 13284, 13285.were selected from the ORFs having the nucleotide sequence represented by SEQ ID NO: 670.

The probes having the nucleotide sequence represented by SEQ ID NOS: 13286, 13287, 13288, 13289, 13290, 13291, 13292, 13293, 13294, 13295, 13296 were selected from the ORFs having the nucleotide sequence represented by SEQ ID NO: 671.

The probes having the nucleotide sequence represented by SEQ ID NOS: 13297, 13298, 13299, 13300, 13301, 13302, 13303, 13304, 13305, 13306, 13307 were selected from the ORFs having the nucleotide sequence represented by SEQ ID NO: 672.

The probes having the nucleotide sequence represented by SEQ ID NOS: 13308, 13309, 13310, 13311, 13312, 13313, 13314, 13315, 13316, 13317, 13318 were selected from the ORFs having the nucleotide sequence represented by SEQ ID NO: 673.

The probes having the nucleotide sequence represented by SEQ ID NOS: 13319, 13320, 13321, 13322, 13323, 13324, 13325, 13326, 13327, 13328, 13329 were selected from the ORFs having the nucleotide sequence represented by SEQ ID NO: 674.

The probes having the nucleotide sequence represented by SEQ ID NOS: 13330, 13331, 13332, 13333, 13334, 13335, 13336, 13337, 13338, 13339, 13340 were selected from the ORFs having the nucleotide sequence represented by SEQ ID NO: 675.

The probes having the nucleotide sequence represented by SEQ ID NOS: 13341, 13342, 13343, 13344, 13345, 13346, 13347, 13348, 13349, 13350, 13351 were selected from the ORFs having the nucleotide sequence represented by SEQ ID NO: 676.

The probes having the nucleotide sequence represented by SEQ ID NOS: 13352, 13353, 13354, 13355, 13356, 13357, 13358, 13359, 13360, 13361, 13362 were selected from the ORFs having the nucleotide sequence represented by SEQ ID NO: 677.

The probes having the nucleotide sequence represented by SEQ ID NOS: 13363, 13364, 13365, 13366, 13367, 13368, 13369, 13370, 13371, 13372, 13373 were selected from the ORFs having the nucleotide sequence represented by SEQ ID NO: 678.

The probes having the nucleotide sequence represented by SEQ ID NOS: 13374, 13375, 13376, 13377, 13378, 13379, 13380, 13381, 13382, 13383, 13384 were selected from the ORFs having the nucleotide sequence represented by SEQ ID NO: 679.

The probes having the nucleotide sequence represented by SEQ ID NOS: 13385, 13386, 13387, 13388, 13389, 13390, 13391, 13392, 13393, 13394, 13395 were selected from the ORFs having the nucleotide sequence represented by SEQ ID NO: 680.

The probes having the nucleotide sequence represented by SEQ ID NOS: 1339.6, 13397, 13398, 13399, 13400, 13401, 13402, 13403, 13404, 13405, 13406 were selected from the ORFs having the nucleotide sequence represented by SEQ ID NO: 681.

The probes having the nucleotide sequence represented by SEQ ID NOS: 13407, 13408, 13409, 13410, 13411, 13412, 13413, 13414, 13415, 13416, 13417, 13418, 13419, 13420, 13421, 13422, 13423, 13424, 13425, 13426, 13427, 13428 were selected from the ORFs having the nucleotide sequence represented by SEQ ID NO: 682.

The probes having the nucleotide sequence represented by SEQ ID NOS: 13429, 13430, 13431, 13432, 13433, 13434, 13435, 13436, 13437, 13438, 13439 were selected from the ORFs having the nucleotide sequence represented by SEQ ID NO: 683.

The probes having the nucleotide sequence represented by SEQ ID NOS: 13440, 13441, 13442, 13443, 13444, 13445, 13446, 13447, 13448, 13449, 13450 were selected from the ORFs having the nucleotide sequence represented by SEQ ID NO: 684.

The probes having the nucleotide sequence represented by SEQ ID NOS: 13451, 13452, 13453, 13454, 13455, 13456, 13457, 13458, 13459, 13460, 13461 were selected from the ORFs having the nucleotide sequence represented by SEQ ID NO: 685.

The probes having the nucleotide sequence represented by SEQ ID NOS: 13462, 13463, 13464, 13465, 13466, 13467, 13468, 13469, 13470, 13471, 13472 were selected from the ORFs having the nucleotide sequence represented by SEQ ID NO: 686.

The probes having the nucleotide sequence represented by SEQ ID NOS: 13473, 13474, 13475, 13476, 13477, 13478, 13479, 13480, 13481, 13482, 13483 were selected from the ORFs having the nucleotide sequence represented by SEQ ID NO: 687.

The probes having the nucleotide sequence represented by SEQ ID NOS: 13484, 13485, 13486, 13487, 13488, 13489, 13490, 13491, 13492, 13493, 13494 were selected from the ORFs having the nucleotide sequence represented by SEQ-ID NO: 688.

The probes having the nucleotide sequence represented by SEQ ID NOS: 13495, 13496, 13497, 13498, 13499, 13500, 13501, 13502, 13503, 13504, 13505 were selected from the ORFs having the nucleotide sequence represented by SEQ ID NO: 689.

The probes having the nucleotide sequence represented by SEQ ID NOS: 13506, 13507, 13508, 13509, 13510, 13511, 13512, 13513, 13514, 13515, 13516 were selected from the ORFs having the nucleotide sequence represented by SEQ ID NO: 690.

The probes having the nucleotide sequence represented by SEQ ID NOS: 13517, 13518, 13519, 13520, 13521, 13522, 13523, 13524, 13525, 13526, 13527 were selected from the ORFs having the nucleotide sequence represented by SEQ ID NO: 691.

The probes having the nucleotide sequence represented by SEQ ID NOS: 13528, 13529, 13530, 13531, 13532, 13533, 13534, 13535, 13536, 13537, 13538 were selected from the ORFs having the nucleotide sequence represented by SEQ ID NO: 692.

The probes having the nucleotide sequence represented by SEQ ID NOS: 13539, 13540, 13541, 13542, 13543, 13544, 13545, 13546, 13547, 13548, 13549 were selected from the ORFs having the nucleotide sequence represented by SEQ ID NO: 693.

The probes having the nucleotide sequence represented by SEQ ID NOS: 13550, 13551, 13552, 13553, 13554, 13555, 13556, 13557, 13558, 13559, 13560 were selected from the ORFs having the nucleotide sequence represented by SEQ ID NO: 694.

The probes having the nucleotide sequence represented by SEQ ID NOS: 13561, 13562, 13563, 13564, 13565, 13566, 13567, 13568, 13569, 13570, 13571 were selected from the ORFs having the nucleotide sequence represented by SEQ ID NO: 695.

The probes having the nucleotide sequence represented by SEQ ID NOS: 13572, 13573, 13574, 13575, 13576, 13577, 13578, 13579, 13580, 13581, 13582 were selected from the ORFs having the nucleotide sequence represented by SEQ ID NO: 696.

The probes having the nucleotide sequence represented by SEQ ID NOS: 13583, 13584, 13585, 13586, 13587, 13588, 13589, 13590, 13591, 13592, 13593 were selected from the ORFs having the nucleotide sequence represented by SEQ ID NO: 697.

The probes having the nucleotide sequence represented by SEQ ID NOS: 13594, 13595, 13596, 13597, 13598, 13599, 13600, 13601, 13602, 13603, 13604 were selected from the ORFs having the nucleotide sequence represented by SEQ ID NO: 698.

The probes having the nucleotide sequence represented by SEQ ID NOS: 13605, 13606, 13607, 13608, 13609, 13610, 13611, 13612, 13613, 13614, 13615 were selected from the ORFs having the nucleotide sequence represented by SEQ ID NO: 699.

The probes having the nucleotide sequence represented by SEQ ID NOS: 13616, 13617, 13618, 13619, 13620, 13621, 13622, 13623, 13624, 13625, 13626 were selected from the ORFs having the nucleotide sequence represented by SEQ ID NO: 700.

The probes having the nucleotide sequence represented by SEQ ID NOS: 13627, 13628, 13629, 13630, 13631, 13632, 13633, 13634, 13635, 13636, 13637 were selected from the ORFs having the nucleotide sequence represented by SEQ ID NO: 701.

The probes having the nucleotide sequence represented by SEQ ID NOS: 13638, 13639, 13640, 13641, 13642, 13643, 13644, 13645, 13646, 13647, 13648 were selected from the ORFs having the nucleotide sequence represented by SEQ ID NO: 702.

The probes having the nucleotide sequence represented by SEQ ID NOS: 13649, 13650, 13651, 13652, 13653, 13654, 13655, 13656, 13657, 13658, 13659 were selected from the ORFs having the nucleotide sequence represented by SEQ ID NO: 703.

The probes having the nucleotide sequence represented by SEQ ID NOS: 13660, 13661, 13662, 13663, 13664, 13665, 13666, 13667, 13668, 13669, 13670 were selected from the ORFs having the nucleotide sequence represented by SEQ ID NO: 704.

The probes having the nucleotide sequence represented by SEQ ID NOS: 13671, 13672, 13673, 13674, 13675, 13676, 13677, 13678, 13679, 13680, 13681 were selected from the ORFs having the nucleotide sequence represented by SEQ ID NO: 705.

The probes having the nucleotide sequence represented by SEQ ID NOS: 13682, 13683, 13684, 13685, 13686, 13687, 13688, 13689, 13690, 13691, 13692 were selected from the ORFs having the nucleotide sequence represented by SEQ ID NO: 706.

The probes having the nucleotide sequence represented by SEQ ID NOS: 13693, 13694, 13695, 13696, 13697, 13698, 13699, 13700, 13701, 13702, 13703 were selected from the ORFs having the nucleotide sequence represented by SEQ ID NO: 707.

The probes having the nucleotide sequence represented by SEQ ID NOS: 13704, 13705, 13706, 13707, 13708, 13709, 13710, 13711, 13712, 13713, 13714 were selected from the ORFs having the nucleotide sequence represented by SEQ ID NO: 708.

The probes having the nucleotide sequence represented by SEQ ID NOS: 13715, 13716, 13717, 13718, 13719, 13720, 13721, 13722, 13723, 13724, 13725 were selected from the ORFs having the nucleotide sequence represented by SEQ ID NO: 709.

The probes having the nucleotide sequence represented by SEQ ID NOS: 13726, 13727, 13728, 13729, 13730, 13731, 13732, 13733, 13734, 13735, 13736 were selected from the ORFs having the nucleotide sequence represented by SEQ ID NO: 710.

The probes having the nucleotide sequence represented by SEQ ID NOS: 13737, 13738, 13739, 13740, 13741, 13742, 13743, 13744, 13745, 13746, 13747 were selected from the ORFs having the nucleotide sequence represented by SEQ ID NO: 711.

The probes having the nucleotide sequence represented by SEQ ID NOS: 13748, 13749, 13750, 13751, 13752, 13753, 13754, 13755, 13756, 13757, 13758 were selected from the ORFs having the nucleotide sequence represented by SEQ ID NO: 712.

The probes having the nucleotide sequence represented by SEQ ID NOS: 13759, 13760, 13761, 13762, 13763, 13764, 13765, 13766, 13767, 13768, 13769 were selected from the ORFs having the nucleotide sequence represented by SEQ ID NO: 713.

The probes having the nucleotide sequence represented by SEQ ID NOS: 13770, 13771, 13772, 13773, 13774, 13775, 13776, 13777, 13778, 13779, 13780 were selected from the ORFs having the nucleotide sequence represented by SEQ ID NO: 714.

The probes having the nucleotide sequence represented by SEQ ID NOS: 13781, 13782, 13783, 13784, 13785, 13786, 13787, 13788, 13789, 13790, 13791 were selected from the ORFs having the nucleotide sequence represented by SEQ ID NO: 715.

The probes having the nucleotide sequence represented by SEQ ID NOS: 13792, 13793, 13794, 13795, 13796, 13797, 13798, 13799, 13800, 13801, 13802 were selected from the ORFs having the nucleotide sequence represented by SEQ ID NO: 716.

The probes having the nucleotide sequence represented by SEQ ID NOS: 13803, 13804, 13805, 13806, 13807, 13808, 13809, 13810, 13811, 13812, 13813 were selected from the ORFs having the nucleotide sequence represented by SEQ ID NO: 717.

The probes having the nucleotide sequence represented by SEQ ID NOS: 13814, 13815, 13816, 13817, 13818, 13819, 13820, 13821, 13822, 13823, 13824 were selected from the ORFs having the nucleotide sequence represented by SEQ ID NO: 718.

The probes having the nucleotide sequence represented by SEQ ID NOS: 13825, 13826, 13827, 13828, 13829, 13830, 13831, 13832, 13833, 13834, 13835 were selected from the ORFs having the nucleotide sequence represented by SEQ ID NO: 719.

The probes having the nucleotide sequence represented by SEQ ID NOS: 13836, 13837, 13838, 13839, 13840, 13841, 13842, 13843, 13844, 13845, 13846 were selected from the ORFs having the nucleotide sequence represented by SEQ ID NO: 720.

The probes having the nucleotide sequence represented by SEQ ID NOS: 13847, 13848, 13849, 13850, 13851, 13852, 13853, 13854, 13855, 13856, 13857 were selected from the ORFs having the nucleotide sequence represented by SEQ ID NO: 721.

The probes having the nucleotide sequence represented by SEQ ID NOS: 13858, 13859, 13860, 13861, 13862, 13863, 13864, 13865, 13866, 13867, 13868 were selected from the ORFs having the nucleotide sequence represented by SEQ ID NO: 722.

The probes having the nucleotide sequence represented by SEQ ID NOS: 13869, 13870, 13871, 13872, 13873, 13874, 13875, 13876, 13877, 13878, 13879 were selected from the ORFs having the nucleotide sequence represented by SEQ ID NO: 723.

The probes having the nucleotide sequence represented by SEQ ID NOS: 13880, 13881, 13882, 13883, 13884, 13885, 13886, 13887, 13888, 13889, 13890 were selected from the ORFs having the nucleotide sequence represented by SEQ ID NO: 724.

The probes having the nucleotide sequence represented by SEQ ID NOS: 13891, 13892, 13893, 13894, 13895, 13896, 13897, 13898, 13899, 13900, 13901 were selected from the ORFs having the nucleotide sequence represented by SEQ ID NO: 725.

The probes having the nucleotide sequence represented by SEQ ID NOS: 13902, 13903, 13904, 13905, 13906, 13907, 13908, 13909, 13910, 13911, 13912 were selected from the ORFs having the nucleotide sequence represented by SEQ ID NO: 726.

The probes having the nucleotide sequence represented by SEQ ID NOS: 13913, 13914, 13915, 13916, 13917, 13918, 13919, 13920, 13921, 13922, 13923 were selected from the ORFs having the nucleotide sequence represented by SEQ ID NO: 727.

The probes having the nucleotide sequence represented by SEQ ID NOS: 13924, 13925, 13926, 13927, 13928, 13929, 13930, 13931, 13932, 13933, 13934 were selected from the ORFs having the nucleotide sequence represented by SEQ ID NO: 728.

The probes having the nucleotide sequence represented by SEQ ID NOS: 13935, 13936, 13937, 13938, 13939, 13940, 13941, 13942, 13943, 13944, 13945 were selected from the ORFs having the nucleotide sequence represented by SEQ ID NO: 729.

The probes having the nucleotide sequence represented by SEQ ID NOS: 13946, 13947, 13948, 13949, 13950, 13951, 13952, 13953, 13954, 13955, 13956 were selected from the ORFs having the nucleotide sequence represented by SEQ ID NO: 730.

The probes having the nucleotide sequence represented by SEQ ID NOS: 13957, 13958, 13959, 13960, 13961, 13962, 13963, 13964, 13965, 13966, 13967 were selected from the ORFs having the nucleotide sequence represented by SEQ ID NO: 731.

The probes having the nucleotide sequence represented by SEQ ID NOS: 13968, 13969, 13970, 13971, 13972, 13973, 13974, 13975, 13976, 13977, 13978 were selected from the ORFs having the nucleotide sequence represented by SEQ ID NO: 732.

The probes having the nucleotide sequence represented by SEQ ID NOS: 13979, 13980, 13981, 13982, 13983, 13984, 13985, 13986, 13987, 13988, 13989 were selected from the ORFs having the nucleotide sequence represented by SEQ ID NO: 733.

The probes having the nucleotide sequence represented by SEQ ID NOS: 13990, 13991, 13992, 13993, 13994, 13995, 13996, 13997, 13998, 13999, 14000 were selected from the ORFs having the nucleotide sequence represented by SEQ ID NO: 734.

The probes having the nucleotide sequence represented by SEQ ID NOS: 14001, 14002, 14003, 14004, 14005, 14006, 14007, 14008, 14009, 14010, 14011 were selected from the ORFs having the nucleotide sequence represented by SEQ ID NO: 735.

The probes having the nucleotide sequence represented by SEQ ID NOS: 14012, 14013, 14014, 14015, 14016, 14017, 14018, 14019, 14020, 14021, 14022 were selected from the ORFs having the nucleotide sequence represented by SEQ ID NO: 736.

The probes having the nucleotide sequence represented by SEQ ID NOS: 14023, 14024, 14025, 14026, 14027, 14028, 14029, 14030, 14031, 14032, 14033 were selected from the ORFs having the nucleotide sequence represented by SEQ ID NO: 737.

The probes having the nucleotide sequence represented by SEQ ID NOS: 14034, 14035, 14036, 14037, 14038, 14039, 14040, 14041, 14042, 14043, 14044, 14045, 14046, 14047, 14048, 14049, 14050, 14051, 14052, 14053, 14054, 14055 were selected from the ORFs having the nucleotide sequence represented by SEQ ID NO: 738.

The probes having the nucleotide sequence represented by SEQ ID NOS: 14056, 14057, 14058, 14059, 14060, 14061, 14062, 14063, 14064, 14065, 14066 were selected from the ORFs having the nucleotide sequence represented by SEQ ID NO: 739.

The probes having the nucleotide sequence represented by SEQ ID NOS: 14067, 14068, 14069, 14070, 14071, 14072, 14073, 14074, 14075, 14076, 14077 were selected from the ORFs having the nucleotide sequence represented by SEQ ID NO: 740.

The probes having the nucleotide sequence represented by SEQ ID NOS: 14078, 14079, 14080, 14081, 14082, 14083, 14084, 14085, 14086, 14087, 14088 were selected from the. ORFs having the nucleotide sequence represented by SEQ ID NO: 741.

The probes having the nucleotide sequence represented by SEQ ID NOS: 14089, 14090, 14091, 14092, 14093, 14094, 14095, 14096, 14097, 14098, 14099 were selected from the ORFs having the nucleotide sequence represented by SEQ ID NO: 742.

The probes having the nucleotide sequence represented by SEQ ID NOS: 14100, 14101, 14102, 14103, 14104, 14105, 14106, 14107, 14108, 14109, 14110 were selected from the ORFs having the nucleotide sequence represented by SEQ ID NO: 743.

The probes having the nucleotide sequence represented by SEQ ID NOS: 14111, 14112, 14113, 14114, 14115, 14116, 14117, 14118, 14119, 14120, 14121 were selected from the ORFs having the nucleotide sequence represented by SEQ ID NO: 744.

The probes having the nucleotide sequence represented by SEQ ID NOS: 14122, 14123, 14124, 14125, 14126, 14127, 14128, 14129, 14130, 14131, 14132 were selected from the ORFs having the nucleotide sequence represented by SEQ ID NO: 745.

The probes having the nucleotide sequence represented by SEQ ID NOS: 14133, 14134, 14135, 14136, 14137, 14138, 14139, 14140, 14141, 14142, 14143 were selected from the ORFs having the nucleotide sequence represented by SEQ ID NO: 746.

The probes having the nucleotide sequence represented by SEQ ID NOS: 14144, 14145, 14146, 14147, 14148, 14149, 14150, 14151, 14152, 14153, 14154 were selected from the ORFs having the nucleotide sequence represented by SEQ ID NO: 747.

The probes having the nucleotide sequence represented by SEQ ID NOS: 14155, 14156, 14157, 14158, 14159, 14160, 14161, 14162, 14163, 14164, 14165 were selected from the ORFs having the nucleotide sequence represented by SEQ ID NO: 748.

The probes having the nucleotide sequence represented by SEQ ID NOS: 14166, 14167, 14168, 14169, 14170, 14171, 14172, 14173, 14174, 14175, 14176 were selected from the ORFs having the nucleotide sequence represented by SEQ ID NO: 749.

The probes having the nucleotide sequence represented by SEQ ID NOS: 14177, 14178, 14179, 14180, 14181, 14182, 14183, 14184, 14185, 14186, 14187 were selected from the ORFs having the nucleotide sequence represented by SEQ ID NO: 750.

The probes having the nucleotide sequence represented by SEQ ID NOS: 14188, 14189, 14190, 14191, 14192, 14193, 14194, 14195, 14196, 14197, 14198 were selected from the ORFs having the nucleotide sequence represented by SEQ ID NO: 751.

The probes having the nucleotide sequence represented by SEQ ID NOS: 14199, 14200, 14201, 14202, 14203, 14204, 14205, 14206, 14207, 14208, 14209 were selected from the ORFs having the nucleotide sequence represented by SEQ ID NO: 752.

The probes having the nucleotide sequence represented by SEQ ID NOS: 14210, 14211, 14212, 14213, 14214, 14215, 14216, 14217, 14218, 14219, 14220 were selected from the ORFs having the nucleotide sequence represented by SEQ ID NO: 753.

The probes having the nucleotide sequence represented by SEQ ID NOS: 14221, 14222, 14223, 14224, 14225, 14226, 14227, 14228, 14229, 14230, 14231 were selected from the ORFs having the nucleotide sequence represented by SEQ ID NO: 754.

The probes having the nucleotide sequence represented by SEQ ID NOS: 14232, 14233, 14234, 14235, 14236, 14237, 14238, 14239, 14240, 14241, 14242 were selected from the ORFs having the nucleotide sequence represented by SEQ ID NO: 755.

The probes having the nucleotide sequence represented by SEQ ID NOS: 14243, 14244, 14245, 14246, 14247, 14248, 14249, 14250, 14251, 14252, 14253 were selected from the ORFs having the nucleotide sequence represented by SEQ ID NO: 756.

The probes having the nucleotide sequence represented by SEQ ID NOS: 14254, 14255, 14256, 14257, 14258, 14259, 14260, 14261, 14262, 14263, 14264 were selected from the ORFs having the nucleotide sequence represented by SEQ ID NO: 757.

The probes having the nucleotide sequence represented by SEQ ID NOS: 14265, 14266, 14267, 14268, 14269, 14270, 14271, 14272, 14273, 14274, 14275 were selected from the ORFs having the nucleotide sequence represented by SEQ ID NO: 758.

The probes having the nucleotide sequence represented by SEQ ID NOS: 14276, 14277, 14278, 14279, 14280, 14281, 14282, 14283, 14284, 14285, 14286 were selected from the ORFs having the nucleotide sequence represented by SEQ ID NO: 759.

The probes having the nucleotide sequence represented by SEQ ID NOS: 14287, 14288, 14289, 14290, 14291, 14292, 14293, 14294, 14295, 14296, 14297 were selected from the ORFs having the nucleotide sequence represented by SEQ ID NO: 760.

The probes having the nucleotide sequence represented by SEQ ID NOS: 14298, 14299, 14300, 14301, 14302, 14303, 14304, 14305, 14306, 14307, 14308 were selected from the ORFs having the nucleotide sequence represented by SEQ ID NO: 761.

The probes having the nucleotide sequence represented by SEQ ID NOS: 14309, 14310, 14311, 14312, 14313, 14314, 14315, 14316, 14317, 14318, 14319 were selected from the ORFs having the nucleotide sequence represented by SEQ ID NO: 762.

The probes having the nucleotide sequence represented by SEQ ID NOS: 14320, 14321, 14322, 14323, 14324, 14325, 14326, 14327, 14328, 14329, 14330 were selected from the ORFs having the nucleotide sequence represented by SEQ ID NO: 763.

The probes having the nucleotide sequence represented by SEQ ID NOS: 14331, 14332, 14333, 14334, 14335, 14336, 14337, 14338, 14339, 14340, 14341 were selected from the ORFs having the nucleotide sequence represented by SEQ ID NO: 764.

The probes having the nucleotide sequence represented by SEQ ID NOS: 14342, 14343, 14344, 14345, 14346, 14347, 14348, 14349, 14350, 14351, 14352, 14353, 14354, 14355, 14356, 14357, 14358, 14359, 14360, 14361, 14362, 14363 were selected from the ORFs having the nucleotide sequence represented by SEQ ID NO: 765.

The probes having the nucleotide sequence represented by SEQ ID NOS: 14364, 14365, 14366, 14367, 14368, 14369, 14370, 14371, 14372, 14373, 14374 were selected from the ORFs having the nucleotide sequence represented by SEQ ID NO: 766.

The probes having the nucleotide sequence represented by SEQ ID NOS: 14375, 14376, 14377, 14378, 14379, 14380, 14381, 14382, 14383, 14384, 14385 were selected from the ORFs having the nucleotide sequence represented by SEQ ID NO: 767.

The probes having the nucleotide sequence represented by SEQ ID NOS: 14386, 14387, 14388, 14389, 14390, 14391, 14392, 14393, 14394, 14395, 14396 were selected from the ORFs having the nucleotide sequence represented by SEQ ID NO: 768.

The probes having the nucleotide sequence represented by SEQ ID NOS: 14397, 14398, 14399, 14400, 14401, 14402, 14403, 14404, 14405, 14406, 14407 were selected from the ORFs having the nucleotide sequence represented by SEQ ID NO: 769.

The probes having the nucleotide sequence represented by SEQ ID NOS: 14408, 14409, 14410, 14411, 14412, 14413, 14414, 14415, 14416, 14417, 14418 were selected from the ORFs having the nucleotide sequence represented by SEQ ID NO: 770.

The probes having the nucleotide sequence represented by SEQ ID NOS: 14419, 14420, 14421, 14422, 14423, 14424, 14425, 14426, 14427, 14428, 14429 were selected from the ORFs having the nucleotide sequence represented by SEQ ID NO: 771.

The probes having the nucleotide sequence represented by SEQ ID NOS: 14430, 14431, 14432, 14433, 14434, 14435, 14436, 14437, 14438, 14439, 14440 were selected from the ORFs having the nucleotide sequence represented by SEQ ID NO: 772.

The probes having the nucleotide sequence represented by SEQ ID NOS: 14441, 14442, 14443, 14444, 14445, 14446, 14447, 14448, 14449, 14450, 14451 were selected from the ORFs having the nucleotide sequence represented by SEQ ID NO: 773.

The probes having the nucleotide sequence represented by SEQ ID NOS: 14452, 14453, 14454, 14455, 14456, 14457, 14458, 14459, 14460, 14461, 14462 were selected from the ORFs having the nucleotide sequence represented by SEQ ID NO: 774.

The probes having the nucleotide sequence represented by SEQ ID NOS: 14463, 14464, 14465, 14466, 14467, 14468, 14469, 14470, 14471, 14472, 14473 were selected from the ORFs having the nucleotide sequence represented by SEQ ID NO: 775.

The probes having the nucleotide sequence represented by SEQ ID NOS: 14474, 14475, 14476, 14477, 14478, 14479, 14480, 14481, 14482, 14483, 14484 were selected from the ORFs having the nucleotide sequence represented by SEQ ID NO: 776.

The probes having the nucleotide sequence represented by SEQ ID NOS: 14485, 14486, 14487, 14488, 14489, 14490, 14491, 14492, 14493, 14494, 14495 were selected from the ORFs having the nucleotide sequence represented by SEQ ID NO: 777.

The probes having the nucleotide sequence represented by SEQ ID NOS: 14496, 14497, 14498, 14499, 14500, 14501, 14502, 14503, 14504, 14505, 14506 were selected from the ORFs having the nucleotide sequence represented by SEQ ID NO: 778.

The probes having the nucleotide sequence represented by SEQ ID NOS: 14507, 14508, 14509, 14510, 14511, 14512, 14513, 14514, 14515, 14516, 14517 were selected from the ORFs having the nucleotide sequence represented by SEQ ID NO: 779.

The probes having the nucleotide sequence represented by SEQ ID NOS: 14518, 14519, 14520, 14521, 14522, 14523, 14524, 14525, 14526, 14527, 14528 were selected from the ORFs having the nucleotide sequence represented by SEQ ID NO: 780.

The probes having the nucleotide sequence represented by SEQ ID NOS: 14529, 14530, 14531, 14532, 14533, 14534, 14535, 14536, 14537, 14538, 14539 were selected from the ORFs having the nucleotide sequence represented by SEQ ID NO: 781.

The probes having the nucleotide sequence represented by SEQ ID NOS: 14540, 14541, 14542, 14543, 14544, 14545, 14546, 14547, 14548, 14549, 14550 were selected from the ORFs having the nucleotide sequence represented by SEQ ID NO: 782.

The probes having the nucleotide sequence represented by SEQ ID NOS: 14551, 14552, 14553, 14554, 14555, 14556, 14557, 14558, 14559, 14560, 14561 were selected from the ORFs having the nucleotide sequence represented by SEQ ID NO: 783.

The probes having the nucleotide sequence represented by SEQ ID NOS: 14562, 14563, 14564, 14565, 14566, 14567, 14568, 14569, 14570, 14571, 14572 were selected from the ORFs having the nucleotide sequence represented by SEQ ID NO: 784.

The probes having the nucleotide sequence represented by SEQ ID NOS: 14573, 14574, 14575, 14576, 14577, 14578, 14579, 14580, 14581, 14582, 14583 were selected from the ORFs having the nucleotide sequence represented by SEQ ID NO: 785.

The probes having the nucleotide sequence represented by SEQ ID NOS: 14584, 14585, 14586, 14587, 14588, 14589, 14590, 14591, 14592, 14593, 14594 were selected from the ORFs having the nucleotide sequence represented by SEQ ID NO: 786.

The probes having the nucleotide sequence represented by SEQ ID NOS: 14595, 14596, 14597, 14598, 14599, 14600, 14601, 14602, 14603, 14604, 14605 were selected from the ORFs having the nucleotide sequence represented by SEQ ID NO: 787.

The probes having the nucleotide sequence represented by SEQ ID NOS: 14606, 14607, 14608, 14609, 14610, 14611, 14612, 14613, 14614, 14615, 14616 were selected from the ORFs having the nucleotide sequence represented by SEQ ID NO: 788.

The probes having the nucleotide sequence represented by SEQ ID NOS: 14617, 14618, 14619, 14620, 14621, 14622, 14623, 14624, 14625, 14626, 14627 were selected from the ORFs having the nucleotide sequence represented by SEQ ID NO: 789.

The probes having the nucleotide sequence represented by SEQ ID NOS: 14628, 14629, 14630, 14631, 14632, 14633, 14634, 14635, 14636, 14637, 14638 were selected from the ORFs having the nucleotide sequence represented by SEQ ID NO: 790.

The probes having the nucleotide sequence represented by SEQ ID NOS: 14639, 14640, 14641, 14642, 14643, 14644, 14645, 14646, 14647, 14648, 14649 were selected from the ORFs having the nucleotide sequence represented by SEQ ID NO: 791.

The probes having the nucleotide sequence represented by SEQ ID NOS: 14650, 14651, 14652, 14653, 14654, 14655, 14656, 14657, 14658, 14659, 14660 were selected from the ORFs having the nucleotide sequence represented by SEQ ID NO: 792.

The probes having the nucleotide sequence represented by SEQ ID NOS: 14661, 14662, 14663, 14664, 14665, 14666, 14667, 14668, 14669, 14670, 14671 were selected from the ORFs having the nucleotide sequence represented by SEQ ID NO: 793.

The probes having the nucleotide sequence represented by SEQ ID NOS: 14672, 14673, 14674, 14675, 14676, 14677, 14678, 14679, 14680, 14681, 14682 were selected from the ORFs having the nucleotide sequence represented by SEQ ID NO: 794.

The probes having the nucleotide sequence represented by SEQ ID NOS: 14705, 14706, 14707, 14708, 14709, 14710, 14711, 14712, 14713, 14714, 14715 were selected from the ORFs having the nucleotide sequence represented by SEQ ID NO: 795.

The probes having the nucleotide sequence represented by SEQ ID NOS: 14683, 14684, 14685, 14686, 14687, 14688, 14689, 14690, 14691, 14692, 14693 were selected from the ORFs having the nucleotide sequence represented by SEQ ID NO: 796.

The probes having the nucleotide sequence represented by SEQ ID NOS: 14694, 14695, 14696, 14697, 14698, 14699, 14700, 14701, 14702, 14703, 14704 were selected from the ORFs having the nucleotide sequence represented by SEQ ID NO: 797.

The probes having the nucleotide sequence represented by SEQ ID NOS: 14716, 14717, 14718, 14719, 14720, 14721, 14722, 14723, 14724, 14725, 14726 were selected from the ORFs having the nucleotide sequence represented by SEQ ID NO: 798.

The probes having the nucleotide sequence represented by SEQ ID NOS: 14727, 14728, 14729, 14730, 14731, 14732, 14733, 14734, 14735, 14736, 14737 were selected from the ORFs having the nucleotide sequence represented by SEQ ID NO: 799.

The probes having the nucleotide sequence represented by SEQ ID NOS: 14738, 14739, 14740, 14741, 14742, 14743, 14744, 14745, 14746, 14747, 14748 were selected from the ORFs having the nucleotide sequence represented by SEQ ID NO: 800.

The probes having the nucleotide sequence represented by SEQ ID NOS: 14749, 14750, 14751, 14752, 14753, 14754, 14755, 14756, 14757, 14758, 14759 were selected from the ORFs having the nucleotide sequence represented by SEQ ID NO: 801.

The probes having the nucleotide sequence represented by SEQ ID NOS: 14760, 14761, 14762, 14763, 14764, 14765, 14766, 14767, 14768, 14769, 14770 were selected from the ORFs having the nucleotide sequence represented by SEQ ID NO: 802.

The probes having the nucleotide sequence represented by SEQ ID NOS: 14771, 14772, 14773, 14774, 14775, 14776, 14777, 14778, 14779, 14780, 14781 were selected from the ORFs having the nucleotide sequence represented by SEQ ID NO: 803.

The probes having the nucleotide sequence represented by SEQ ID NOS: 14782, 14783, 14784, 14785, 14786, 14787, 14788, 14789, 14790, 14791, 14792 were selected from the ORFs having the nucleotide sequence represented by SEQ ID NO: 804.

The probes having the nucleotide sequence represented by SEQ ID NOS: 14793, 14794, 14795, 14796, 14797, 14798, 14799, 14800, 14801, 14802, 14803 were selected from the ORFs having the nucleotide sequence represented by SEQ ID NO: 805.

The probes having the nucleotide sequence represented by SEQ ID NOS: 14804, 14805, 14806, 14807, 14808, 14809, 14810, 14811, 14812, 14813, 14814 were selected from the ORFs having the nucleotide sequence represented by SEQ ID NO: 806.

The probes having the nucleotide sequence represented by SEQ ID NOS: 14815, 14816, 14817, 14818, 14819, 14820, 14821, 14822, 14823, 14824, 14825 were selected from the ORFs having the nucleotide sequence represented by SEQ ID NO: 807.

The probes having the nucleotide sequence represented by SEQ ID NOS: 14826, 14827, 14828, 14829, 14830, 14831, 14832, 14833, 14834, 14835, 14836 were selected from the ORFs having the nucleotide sequence represented by SEQ ID NO: 808.

The probes having the nucleotide sequence represented by SEQ ID NOS: 14837, 14838, 14839, 14840, 14841, 14842, 14843, 14844, 14845, 14846, 14847 were selected from the ORFs having the nucleotide sequence represented by SEQ ID NO: 809.

The probes having the nucleotide sequence represented by SEQ ID NOS: 14848, 14849, 14850, 14851, 14852, 14853, 14854, 14855, 14856, 14857, 14858 were selected from the ORFs having the nucleotide sequence represented by SEQ ID NO: 810.

The probes having the nucleotide sequence represented by SEQ ID NOS: 14859, 14860, 14861, 14862, 14863, 14864, 14865, 14866, 14867, 14868, 14869 were selected from the ORFs having the nucleotide sequence represented by SEQ ID NO: 811.

The probes having the nucleotide sequence represented by SEQ ID NOS: 14870, 14871, 14872, 14873, 14874, 14875, 14876, 14877, 14878, 14879, 14880 were selected from the ORFs having the nucleotide sequence represented by SEQ ID NO: 812.

The probes having the nucleotide sequence represented by SEQ ID NOS: 14881, 14882, 14883, 14884, 14885, 14886, 14887, 14888, 14889, 14890, 14891 were selected from the ORFs having the nucleotide sequence represented by SEQ ID NO: 813.

The probes having the nucleotide sequence represented by SEQ ID NOS: 14892, 14893, 14894, 14895, 14896, 14897, 14898, 14899, 14900, 14901, 14902 were selected from the ORFs having the nucleotide sequence represented by SEQ ID NO: 814.

The probes having the nucleotide sequence represented by SEQ ID NOS: 14903, 14904, 14905, 14906, 14907, 14908, 14909, 14910, 14911, 14912, 14913 were selected from the ORFs having the nucleotide sequence represented by SEQ ID NO: 815.

The probes having the nucleotide sequence represented by SEQ ID NOS: 14914, 14915, 14916, 14917, 14918, 14919, 14920, 14921, 14922, 14923, 14924 were selected from the ORFs having the nucleotide sequence represented by SEQ ID NO: 816.

The probes having the nucleotide sequence represented by SEQ ID NOS: 14925, 14926, 14927, 14928, 14929, 14930, 14931, 14932, 14933, 14934, 14935 were selected from the ORFs having the nucleotide sequence represented by SEQ ID NO: 817.

The probes having the nucleotide sequence represented by SEQ ID NOS: 14936, 14937, 14938, 14939, 14940, 14941, 14942, 14943, 14944, 14945, 14946 were selected from the ORFs having the nucleotide sequence represented by SEQ ID NO: 818.

The probes having the nucleotide sequence represented by SEQ ID NOS: 14947, 14948, 14949, 14950, 14951, 14952, 14953, 14954, 14955, 14956, 14957 were selected from the ORFs having the nucleotide sequence represented by SEQ ID NO: 819.

The probes having the nucleotide sequence represented by SEQ ID NOS: 14958, 14959, 14960, 14961, 14962, 14963, 14964, 14965, 14966, 14967, 14968 were selected from the ORFs having the nucleotide sequence represented by SEQ ID NO: 820.

The probes having the nucleotide sequence represented by SEQ ID NOS: 14969, 14970, 14971, 14972, 14973, 14974, 14975, 14976, 14977, 14978, 14979 were selected from the ORFs having the nucleotide sequence represented by SEQ ID NO: 821.

The probes having the nucleotide sequence represented by SEQ ID NOS: 14980, 14981, 14982, 14983, 14984, 14985, 14986, 14987, 14988, 14989, 14990 were selected from the ORFs having the nucleotide sequence represented by SEQ ID NO: 822.

The probes having the nucleotide sequence represented by SEQ ID NOS: 14991, 14992, 14993, 14994, 14995, 14996, 14997, 14998, 14999, 15000, 15001 were selected from the ORFs having the nucleotide sequence represented by SEQ ID NO: 823.

The probes having the nucleotide sequence represented by SEQ ID NOS: 15002, 15003, 15004, 15005, 15006, 15007, 15008, 15009, 15010, 15011, 15012 were selected from the ORFs having the nucleotide sequence represented by SEQ ID NO: 824.

The probes having the nucleotide sequence represented by SEQ ID NOS: 15013, 15014, 15015, 15016, 15017, 15018, 15019, 15020, 15021, 15022, 15023 were selected from the ORFs having the nucleotide sequence represented by SEQ ID NO: 825.

The probes having the nucleotide sequence represented by SEQ ID NOS: 15024, 15025, 15026, 15027, 15028, 15029, 15030, 15031, 15032, 15033, 15034 were selected from the ORFs having the nucleotide sequence represented by SEQ ID NO: 826.

The probes having the nucleotide sequence represented by SEQ ID NOS: 15035, 15036, 15037, 15038, 15039, 15040, 15041, 15042, 15043, 15044, 15045 were selected from the ORFs having the nucleotide sequence represented by SEQ ID NO: 827.

The probes having the nucleotide sequence represented by SEQ ID NOS: 15046, 15047, 15048, 15049, 15050, 15051, 15052, 15053, 15054, 15055, 15056 were selected from the ORFs having the nucleotide sequence represented by SEQ ID NO: 828.

The probes having the nucleotide sequence represented by SEQ ID NOS: 15057, 15058, 15059, 15060, 15061, 15062, 15063, 15064, 15065, 15066, 15067 were selected from the ORFs having the nucleotide sequence represented by SEQ ID NO: 829.

The probes having the nucleotide sequence represented by SEQ ID NOS: 15068, 15069, 15070, 15071, 15072, 15073, 15074, 15075, 15076, 15077, 15078 were selected from the ORFs having the nucleotide sequence represented by SEQ ID NO: 830.

The probes having the nucleotide sequence represented by SEQ ID NOS: 15079, 15080, 15081, 15082, 15083, 15084, 15085, 15086, 15087, 15088, 15089 were selected from the ORFs having the nucleotide sequence represented by SEQ ID NO: 831.

The probes having the nucleotide sequence represented by SEQ ID NOS: 15090, 15091, 15092, 15093, 15094, 15095, 15096, 15097, 15098, 15099, 15100 were selected from the ORFs having the nucleotide sequence represented by SEQ ID NO: 832.

The probes having the nucleotide sequence represented by SEQ ID NOS: 15101, 15102, 15103, 15104, 15105, 15106, 15107, 15108, 15109, 15110, 15111 were selected from the ORFs having the nucleotide sequence represented by SEQ ID NO: 833.

The probes having the nucleotide sequence represented by SEQ ID NOS: 15112, 15113, 15114, 15115, 15116, 15117, 15118, 15119, 15120, 15121, 15122 were selected from the ORFs having the nucleotide sequence represented by SEQ ID NO: 834.

The probes having the nucleotide sequence represented by SEQ ID NOS: 15123, 15124, 15125, 15126, 15127, 15128, 15129, 15130, 15131, 15132, 15133 were selected from the ORFs having the nucleotide sequence represented by SEQ ID NO: 835.

The probes having the nucleotide sequence represented by SEQ ID NOS: 15134, 15135, 15136, 15137, 15138, 15139, 15140, 15141, 15142, 15143, 15144 were selected from the ORFs having the nucleotide sequence represented by SEQ ID NO: 836.

The probes having the nucleotide sequence represented by SEQ ID NOS: 15145, 15146, 15147, 15148, 15149, 15150, 15151, 15152, 15153, 15154, 15155 were selected from the ORFs having the nucleotide sequence represented by SEQ ID NO: 837.

The probes having the nucleotide sequence represented by SEQ ID NOS: 15156, 15157, 15158, 15159, 15160, 15161, 15162, 15163, 15164, 15165, 15166 were selected from the ORFs having the nucleotide sequence represented by SEQ ID NO: 838.

The probes having the nucleotide sequence represented by SEQ ID NOS: 15167, 15168, 15169, 15170, 15171, 15172, 15173, 15174, 15175, 15176, 15177 were selected from the ORFs having the nucleotide sequence represented by SEQ ID NO: 839.

The probes having the nucleotide sequence represented by SEQ ID NOS: 15178, 15179, 15180, 15181, 15182, 15183, 15184, 15185, 15186, 15187, 15188 were selected from the ORFs having the nucleotide sequence represented by SEQ ID NO: 840.

The probes having the nucleotide sequence represented by SEQ ID NOS: 15189, 15190, 15191, 15192, 15193, 15194, 15195, 15196, 15197, 15198, 15199 were selected from the ORFs having the nucleotide sequence represented by SEQ ID NO: 841.

The probes having the nucleotide sequence represented by SEQ ID NOS: 15200, 15201, 15202, 15203, 15204, 15205, 15206, 15207, 15208, 15209, 15210 were selected from the ORFs having the nucleotide sequence represented by SEQ ID NO: 842.

The probes having the nucleotide sequence represented by SEQ ID NOS: 15211, 15212, 15213, 15214, 15215, 15216, 15217, 15218, 15219, 15220, 15221 were selected from the ORFs having the nucleotide sequence represented by SEQ ID NO: 843.

The probes having the nucleotide sequence represented by SEQ ID NOS: 15222, 15223, 15224, 15225, 15226, 15227, 15228, 15229, 15230, 15231, 15232 were selected from the ORFs having the nucleotide sequence represented by SEQ ID NO: 844.

The probes having the nucleotide sequence represented by SEQ ID NOS: 15233, 15234, 15235, 15236, 15237, 15238, 15239, 15240, 15241, 15242, 15243 were selected from the ORFs having the nucleotide sequence represented by SEQ ID NO: 845.

The probes having the nucleotide sequence represented by SEQ ID NOS: 15244, 15245, 15246, 15247, 15248, 15249, 15250, 15251, 15252, 15253, 15254 were selected from the ORFs having the nucleotide sequence represented by SEQ ID NO: 846.

The probes having the nucleotide sequence represented by SEQ ID NOS: 15255, 15256, 15257, 15258, 15259, 15260, 15261, 15262, 15263, 15264, 15265 were selected from the ORFs having the nucleotide sequence represented by SEQ ID NO: 847.

The probes having the nucleotide sequence represented by SEQ ID NOS: 15266, 15267, 15268, 15269, 15270, 15271, 15272, 15273, 15274, 15275, 15276 were selected from the ORFs having the nucleotide sequence represented by SEQ ID NO: 848.

The probes having the nucleotide sequence represented by SEQ ID NOS: 15277, 15278, 15279, 15280, 15281, 15282, 15283, 15284, 15285, 15286, 15287 were selected from the ORFs having the nucleotide sequence represented by SEQ ID NO: 849.

The probes having the nucleotide sequence represented by SEQ ID NOS: 15288, 15289, 15290, 15291, 15292, 15293, 15294, 15295, 15296, 15297, 15298 were selected from the ORFs having the nucleotide sequence represented by SEQ ID NO: 850.

The probes having the nucleotide sequence represented by SEQ ID NOS: 15299, 15300, 15301, 15302, 15303, 15304, 15305, 15306, 15307, 15308, 15309 were selected from the ORFs having the nucleotide sequence represented by SEQ ID NO: 851.

The probes having the nucleotide sequence represented by SEQ ID NOS: 15310, 15311, 15312, 15313, 15314, 15315, 15316, 15317, 15318, 15319, 15320 were selected from the ORFs having the nucleotide sequence represented by SEQ ID NO: 852.

The probes having the nucleotide sequence represented by SEQ ID NOS: 15321, 15322, 15323, 15324, 15325, 15326, 15327, 15328, 15329, 15330, 15331 were selected from the ORFs having the nucleotide sequence represented by SEQ ID NO: 853.

The probes having the nucleotide sequence represented by SEQ ID NOS: 15332, 15333, 15334, 15335, 15336, 15337, 15338, 15339, 15340, 15341, 15342 were selected from the ORFs having the nucleotide sequence represented by SEQ ID NO: 854.

The probes having the nucleotide sequence represented by SEQ ID NOS: 15343, 15344, 15345, 15346, 15347, 15348, 15349, 15350, 15351, 15352, 15353 were selected from the ORFs having the nucleotide sequence represented by SEQ ID NO: 855.

The probes having the nucleotide sequence represented by SEQ ID NOS: 15354, 15355, 15356, 15357, 15358, 15359, 15360, 15361, 15362, 15363, 15364 were selected from the ORFs having the nucleotide sequence represented by SEQ ID NO: 856.

The probes having the nucleotide sequence represented by SEQ ID NOS: 15365, 15366, 15367, 15368, 15369, 15370, 15371, 15372, 15373, 15374, 15375 were selected from the ORFs having the nucleotide sequence represented by SEQ ID NO: 857.

The probes having the nucleotide sequence represented by SEQ ID NOS: 15376, 15377, 15378, 15379, 15380, 15381, 15382, 15383, 15384, 15385, 15386 were selected from the ORFs having the nucleotide sequence represented by SEQ ID NO: 858.

The probes having the nucleotide sequence represented by SEQ ID NOS: 15387, 15388, 15389, 15390, 15391, 15392, 15393, 15394, 15395, 15396, 15397 were selected from the ORFs having the nucleotide sequence represented by SEQ ID NO: 859.

The probes having the nucleotide sequence represented by SEQ ID NOS: 15398, 15399, 15400, 15401, 15402, 15403, 15404, 15405, 15406, 15407, 15408 were selected from the ORFs having the nucleotide sequence represented by SEQ ID NO: 860.

The probes having the nucleotide sequence represented by SEQ ID NOS: 15409, 15410, 15411, 15412, 15413, 15414, 15415, 15416, 15417, 15418, 15419 were selected from the ORFs having the nucleotide sequence represented by SEQ ID NO: 861.

The probes having the nucleotide sequence represented by SEQ ID NOS: 15420, 15421, 15422, 15423, 15424, 15425, 15426, 15427, 15428, 15429, 15430 were selected from the ORFs having the nucleotide sequence represented by SEQ ID NO: 862.

The probes having the nucleotide sequence represented by SEQ ID NOS: 15431, 15432, 15433, 15434, 15435, 15436, 15437, 15438, 15439, 15440, 15441 were selected from the ORFs having the nucleotide sequence represented by SEQ ID NO: 863.

The probes having the nucleotide sequence represented by SEQ ID NOS: 15442, 15443, 15444, 15445, 15446, 15447, 15448, 15449, 15450, 15451, 15452 were selected from the ORFs having the nucleotide sequence represented by SEQ ID NO: 864.

The probes having the nucleotide sequence represented by SEQ ID NOS: 15453, 15454, 15455, 15456, 15457, 15458, 15459, 15460, 15461, 15462, 15463 were selected from the ORFs having the nucleotide sequence represented by SEQ ID NO: 865.

The probes having the nucleotide sequence represented by SEQ ID NOS: 15464, 15465, 15466, 15467, 15468, 15469, 15470, 15471, 15472, 15473, 15474 were selected from the ORFs having the nucleotide sequence represented by SEQ ID NO: 866.

The probes having the nucleotide sequence represented by SEQ ID NOS: 15475, 15476, 15477, 15478, 15479, 15480, 15481, 15482, 15483, 15484, 15485 were selected from the ORFs having the nucleotide sequence represented by SEQ ID NO: 867.

The probes having the nucleotide sequence represented by SEQ ID NOS: 15486, 15487, 15488, 15489, 15490, 15491, 15492, 15493, 15494, 15495, 15496 were selected from the ORFs having the nucleotide sequence represented by SEQ ID NO: 868.

The probes having the nucleotide sequence represented by SEQ ID NOS: 15497, 15498, 15499, 15500, 15501, 15502, 15503, 15504, 15505, 15506, 15507 were selected from the ORFs having the nucleotide sequence represented by SEQ ID NO: 869.

The probes having the nucleotide sequence represented by SEQ ID NOS: 15508, 15509, 15510, 15511, 15512, 15513, 15514, 15515, 15516, 15517, 15518 were selected from the ORFs having the nucleotide sequence represented by SEQ ID NO: 870.

The probes having the nucleotide sequence represented by SEQ ID NOS: 15519, 15520, 15521, 15522, 15523, 15524, 15525, 15526, 15527, 15528, 15529 were selected from the ORFs having the nucleotide sequence represented by SEQ ID NO: 871.

The probes having the nucleotide sequence represented by SEQ ID NOS: 15530, 15531, 15532, 15533, 15534, 15535, 15536, 15537, 15538, 15539, 15540 were selected from the ORFs having the nucleotide sequence represented by SEQ ID NO: 872.

The probes having the nucleotide sequence represented by SEQ ID NOS: 15541, 15542, 15543, 15544, 15545, 15546, 15547, 15548, 15549, 15550, 15551 were selected from the ORFs having the nucleotide sequence represented by SEQ ID NO: 873.

The probes having the nucleotide sequence represented by SEQ ID NOS: 15552, 15553, 15554, 15555, 15556, 15557, 15558, 15559, 15560, 15561, 15562 were selected from the ORFs having the nucleotide sequence represented by SEQ ID NO: 874.

The probes having the nucleotide sequence represented by SEQ ID NOS: 15563, 15564, 15565, 15566, 15567, 15568, 15569, 15570, 15571, 15572, 15573 were selected from the ORFs having the nucleotide sequence represented by SEQ ID NO: 875.

The probes having the nucleotide sequence represented by SEQ ID NOS: 15574, 15575, 15576, 15577, 15578, 15579, 15580, 15581, 15582, 15583, 15584 were selected from the ORFs having the nucleotide sequence represented by SEQ ID NO: 876.

The probes having the nucleotide sequence represented by SEQ ID NOS: 15585, 15586, 15587, 15588, 15589, 15590, 15591, 15592, 15593, 15594, 15595 were selected from the ORFs having the nucleotide sequence represented by SEQ ID NO: 877.

The probes having the nucleotide sequence represented by SEQ ID NOS: 15596, 15597, 15598, 15599, 15600, 15601, 15602, 15603, 15604, 15605, 15606 were selected from the ORFs having the nucleotide sequence represented by SEQ ID NO: 878.

The probes having the nucleotide sequence represented by SEQ ID NOS: 15607, 15608, 15609, 15610, 15611, 15612, 15613, 15614, 15615, 15616, 15617 were selected from the ORFs having the nucleotide sequence represented by SEQ ID NO: 879.

The probes having the nucleotide sequence represented by SEQ ID NOS: 15618, 15619, 15620, 15621, 15622, 15623, 15624, 15625, 15626, 15627, 15628 were selected from the ORFs having the nucleotide sequence represented by SEQ ID NO: 880.

The probes having the nucleotide sequence represented by SEQ ID NOS: 15629, 15630, 15631, 15632, 15633, 15634, 15635, 15636, 15637, 15638, 15639 were selected from the ORFs having the nucleotide sequence represented by SEQ ID NO: 881.

The probes having the nucleotide sequence represented by SEQ ID NOS: 15640, 15641, 15642, 15643, 15644, 15645, 15646, 15647, 15648, 15649, 15650 were selected from the ORFs having the nucleotide sequence represented by SEQ ID NO: 882.

The probes having the nucleotide sequence represented by SEQ ID NOS: 15651, 15652, 15653, 15654, 15655, 15656, 15657, 15658, 15659, 15660, 15661 were selected from the ORFs having the nucleotide sequence represented by SEQ ID NO: 883.

The probes having the nucleotide sequence represented by SEQ ID NOS: 15662, 15663, 15664, 15665, 15666, 15667, 15668, 15669, 15670, 15671, 15672 were selected from the ORFs having the nucleotide sequence represented by SEQ ID NO: 884.

The probes having the nucleotide sequence represented by SEQ ID NOS: 15673, 15674, 15675, 15676, 15677, 15678, 15679, 15680, 15681, 15682, 15683 were selected from the ORFs having the nucleotide sequence represented by SEQ ID NO: 885.

The probes having the nucleotide sequence represented by SEQ ID NOS: 15684, 15685, 15686, 15687, 15688, 15689, 15690, 15691, 15692, 15693, 15694 were selected from the ORFs having the nucleotide sequence represented by SEQ ID NO: 886.

The probes having the nucleotide sequence represented by SEQ ID NOS: 15695, 15696, 15697, 15698, 15699, 15700, 15701, 15702, 15703, 15704, 15705 were selected from the ORFs having the nucleotide sequence represented by SEQ ID NO: 887.

The probes having the nucleotide sequence represented by SEQ ID NOS: 15706, 15707, 15708, 15709, 15710, 15711, 15712, 15713, 15714, 15715, 15716 were selected from the ORFs having the nucleotide sequence represented by SEQ ID NO: 888.

The probes having the nucleotide sequence represented by SEQ ID NOS: 15717, 15718, 15719, 15720, 15721, 15722, 15723, 15724, 15725, 15726, 15727 were selected from the ORFs having the nucleotide sequence represented by SEQ ID NO: 889.

The probes having the nucleotide sequence represented by SEQ ID NOS: 15728, 15729, 15730, 15731, 15732, 15733, 15734, 15735, 15736, 15737, 15738 were selected from the ORFs having the nucleotide sequence represented by SEQ ID NO: 890.

The probes having the nucleotide sequence represented by SEQ ID NOS: 15739, 15740, 15741, 15742, 15743, 15744, 15745, 15746, 15747, 15748, 15749 were selected from the ORFs having the nucleotide sequence represented by SEQ ID NO: 891.

The probes having the nucleotide sequence represented by SEQ ID NOS: 15750, 15751, 15752, 15753, 15754, 15755, 15756, 15757, 15758, 15759, 15760 were selected from the ORFs having the nucleotide sequence represented by SEQ ID NO: 892.

The probes having the nucleotide sequence represented by SEQ ID NOS: 15761, 15762, 15763, 15764, 15765, 15766, 15767, 15768, 15769, 15770, 15771 were selected from the ORFs having the nucleotide sequence represented by SEQ ID NO: 893.

The probes having the nucleotide sequence represented by SEQ ID NOS: 15772, 15773, 15774, 15775, 15776, 15777, 15778, 15779, 15780, 15781, 15782 were selected from the ORFs having the nucleotide sequence represented by SEQ ID NO: 894.

The probes having the nucleotide sequence represented by SEQ ID NOS: 15783, 15784, 15785, 15786, 15787, 15788, 15789, 15790, 15791, 15792, 15793 were selected from the ORFs having the nucleotide sequence represented by SEQ ID NO: 895.

The probes having the nucleotide sequence represented by SEQ ID NOS: 15794, 15795, 15796, 15797, 15798, 15799, 15800, 15801, 15802, 15803, 15804 were selected from the ORFs having the nucleotide sequence represented by SEQ ID NO: 896.

The probes having the nucleotide sequence represented by SEQ ID NOS: 15805, 15806, 15807, 15808, 15809, 15810, 15811, 15812, 15813, 15814, 15815 were selected from the ORFs having the nucleotide sequence represented by SEQ ID NO: 897.

The probes having the nucleotide sequence represented by SEQ ID NOS: 15816, 15817, 15818, 15819, 15820, 15821, 15822, 15823, 15824, 15825, 15826 were selected from the ORFs having the nucleotide sequence represented by SEQ ID NO: 898.

The probes having the nucleotide sequence represented by SEQ ID NOS: 15827, 15828, 15829, 15830, 15831, 15832, 15833, 15834, 15835, 15836, 15837 were selected from the ORFs having the nucleotide sequence represented by SEQ ID NO: 899.

The probes having the nucleotide sequence represented by SEQ ID NOS: 15838, 15839, 15840, 15841, 15842, 15843, 15844, 15845, 15846, 15847, 15848 were selected from the ORFs having the nucleotide sequence represented by SEQ ID NO: 900.

The probes having the nucleotide sequence represented by SEQ ID NOS: 15849, 15850, 15851, 15852, 15853, 15854, 15855, 15856, 15857, 15858, 15859 were selected from the ORFs having the nucleotide sequence represented by SEQ ID NO: 901.

The probes having the nucleotide sequence represented by SEQ ID NOS: 15860, 15861, 15862, 15863, 15864, 15865, 15866, 15867, 15868, 15869, 15870 were selected from the ORFs having the nucleotide sequence represented by SEQ ID NO: 902.

The probes having the nucleotide sequence represented by SEQ ID NOS: 15871, 15872, 15873, 15874, 15875, 15876, 15877, 158.78, 15879, 15880, 15881 were selected from the ORFs having the nucleotide sequence represented by SEQ ID NO: 903.

The probes having the nucleotide sequence represented by SEQ ID NOS: 15882, 15883, 15884, 15885, 15886, 15887, 15888, 15889, 15890, 15891, 15892 were selected from the ORFs having the nucleotide sequence represented by SEQ ID NO: 904.

The probes having the nucleotide sequence represented by SEQ ID NOS: 15893, 15894, 15895, 15896, 15897, 15898, 15899, 15900, 15901, 15902, 15903 were selected from the ORFs having the nucleotide sequence represented by SEQ ID NO: 905.

The probes having the nucleotide sequence represented by SEQ ID NOS: 15904, 15905, 15906, 15907, 15908, 15909, 15910, 15911, 15912, 15913, 15914 were selected from the ORFs having the nucleotide sequence represented by SEQ ID NO: 906.

The probes having the nucleotide sequence represented by SEQ ID NOS: 15915, 15916, 15917, 15918, 15919, 15920, 15921, 15922, 15923, 15924, 15925 were selected from the ORFs having the nucleotide sequence represented by SEQ ID NO: 907.

The probes having the nucleotide sequence represented by SEQ ID NOS: 15926, 15927, 15928, 15929, 15930, 15931, 15932, 15933, 15934, 15935, 15936 were selected from the ORFs having the nucleotide sequence represented by SEQ ID NO: 908.

The probes having the nucleotide sequence represented by SEQ ID NOS: 15937, 15938, 15939, 15940, 15941, 15942, 15943, 15944, 15945, 15946, 15947 were selected from the ORFs having the nucleotide sequence represented by SEQ ID NO: 909.

The probes having the nucleotide sequence represented by SEQ ID NOS: 15948, 15949, 15950, 15951, 15952, 15953, 15954, 15955, 15956, 15957, 15958 were selected from the ORFs having the nucleotide sequence represented by SEQ ID NO: 910.

The probes having the nucleotide sequence represented by SEQ ID NOS: 15959, 15960, 15961, 15962, 15963, 15964, 15965, 15966, 15967, 15968, 15969 were selected from the ORFs having the nucleotide sequence represented by SEQ ID NO: 911.

The probes having the nucleotide sequence represented by SEQ ID NOS: 15970, 15971, 15972, 15973, 15974, 15975, 15976, 15977, 15978, 15979, 15980 were selected from the ORFs having the nucleotide sequence represented by SEQ ID NO: 912.

The probes having the nucleotide sequence represented by SEQ ID NOS: 15981, 15982, 15983, 15984, 15985, 15986, 15987, 15988, 15989, 15990, 15991 were selected from the ORFs having the nucleotide sequence represented by SEQ ID NO: 913.

The probes having the nucleotide sequence represented by SEQ ID NOS: 15992, 15993, 15994, 15995, 15996, 15997, 15998, 15999, 16000, 16001, 16002 were selected from the ORFs having the nucleotide sequence represented by SEQ ID NO: 914.

The probes having the nucleotide sequence represented by SEQ ID NOS: 16003, 16004, 16005, 16006, 16007, 16008, 16009, 16010, 16011, 16012, 16013 were selected from the ORFs having the nucleotide sequence represented by SEQ ID NO: 915.

The probes having the nucleotide sequence represented by SEQ ID NOS: 16014, 16015, 16016, 16017, 16018, 16019, 16020, 16021, 16022, 16023, 16024 were selected from the ORFs having the nucleotide sequence represented by SEQ ID NO: 916.

The probes having the nucleotide sequence represented by SEQ ID NOS: 16025, 16026, 16027, 16028, 16029, 16030, 16031, 16032, 16033, 16034, 16035 were selected from the ORFs having the nucleotide sequence represented by SEQ ID NO: 917.

The probes having the nucleotide sequence represented by SEQ ID NOS: 16036, 16037, 16038, 16039, 16040, 16041, 16042, 16043, 16044, 16045, 16046 were selected from the ORFs having the nucleotide sequence represented by SEQ ID NO: 918.

The probes having the nucleotide sequence represented by SEQ ID NOS: 16047, 16048, 16049, 16050, 16051, 16052, 16053, 16054, 16055, 16056, 16057 were selected from the ORFs having the nucleotide sequence represented by SEQ ID NO: 919.

The probes having the nucleotide sequence represented by SEQ ID NOS: 16058, 16059, 16060, 16061, 16062, 16063, 16064, 16065, 16066, 16067, 16068 were selected from the ORFs having the nucleotide sequence represented by SEQ ID NO: 920.

The probes having the nucleotide sequence represented by SEQ ID NOS: 16069, 16070, 16071, 16072, 16073, 16074, 16075, 16076, 16077, 16078, 16079 were selected from the ORFs having the nucleotide sequence represented by SEQ ID NO: 921.

The probes having the nucleotide sequence represented by SEQ ID NOS: 16080, 16081, 16082, 16083, 16084, 16085, 16086, 16087, 16088, 16089, 16090 were selected from the ORFs having the nucleotide sequence represented by SEQ ID NO: 922.

The probes having the nucleotide sequence represented by SEQ ID NOS: 16091, 16092, 16093, 16094, 16095, 16096, 16097, 16098, 16099, 16100, 16101 were selected from the ORFs having the nucleotide sequence represented by SEQ ID NO: 923.

The probes having the nucleotide sequence represented by SEQ ID NOS: 16102, 16103, 16104, 16105, 16106, 16107, 16108, 16109, 16110, 16111, 16112 were selected from the ORFs having the nucleotide sequence represented by SEQ ID NO: 924.

The probes having the nucleotide sequence represented by SEQ ID NOS: 16113, 16114, 16115, 16116, 16117, 16118, 16119, 16120, 16121, 16122, 16123 were selected from the ORFs having the nucleotide sequence represented by SEQ ID NO: 925.

The probes having the nucleotide sequence represented by SEQ ID NOS: 16124, 16125, 16126, 16127, 16128, 16129, 16130, 16131, 16132, 16133, 16134 were selected from the ORFs having the nucleotide sequence represented by SEQ ID NO: 926.

The probes having the nucleotide sequence represented by SEQ ID NOS: 16135, 16136, 16137, 16138, 16139, 16140, 16141, 16142, 16143, 16144, 16145 were selected from the ORFs having the nucleotide sequence represented by SEQ ID NO: 927.

The probes having the nucleotide sequence represented by SEQ ID NOS: 16146, 16147, 16148, 16149, 16150, 16151, 16152, 16153, 16154, 16155, 16156 were selected from the ORFs having the nucleotide sequence represented by SEQ ID NO: 928.

The probes having the nucleotide sequence represented by SEQ ID NOS: 16157, 16158, 16159, 16160, 16161, 16162, 16163, 16164, 16165, 16166, 16167 were selected from the ORFs having the nucleotide sequence represented by SEQ ID NO: 929.

The probes having the nucleotide sequence represented by SEQ ID NOS: 16168, 16169, 16170, 16171, 16172, 16173, 16174, 16175, 16176, 16177, 16178 were selected from the ORFs having the nucleotide sequence represented by SEQ ID NO: 930.

The probes having the nucleotide sequence represented by SEQ ID NOS: 16179, 16180, 16181, 16182, 16183, 16184, 16185, 16186, 16187, 16188, 16189 were selected from the ORFs having the nucleotide sequence represented by SEQ ID NO: 931.

The probes having the nucleotide sequence represented by SEQ ID NOS: 16190, 16191, 16192, 16193, 16194, 16195, 16196, 16197, 16198, 16199, 16200 were selected from the ORFs having the nucleotide sequence represented by SEQ ID NO: 932.

The probes having the nucleotide sequence represented by SEQ ID NOS: 16201, 16202, 16203, 16204, 16205, 16206, 16207, 16208, 16209, 16210, 16211 were selected from the ORFs having the nucleotide sequence represented by SEQ ID NO: 933.

The probes having the nucleotide sequence represented by SEQ ID NOS: 16212, 16213, 16214, 16215, 16216, 16217, 16218, 16219, 16220, 16221, 16222 were selected from the ORFs having the nucleotide sequence represented by SEQ ID NO: 934.

The probes having the nucleotide sequence represented by SEQ ID NOS: 16223, 16224, 16225, 16226, 16227, 16228, 16229, 16230, 16231, 16232, 16233 were selected from the ORFs having the nucleotide sequence represented by SEQ ID NO: 935.

The probes having the nucleotide sequence represented by SEQ ID NOS: 16234, 16235, 16236, 16237, 16238, 16239, 16240, 16241, 16242, 16243, 16244 were selected from the ORFs having the nucleotide sequence represented by SEQ ID NO: 936.

The probes having the nucleotide sequence represented by SEQ ID NOS: 16245, 16246, 16247, 16248, 16249, 16250, 16251, 16252, 16253, 16254, 16255 were selected from the ORFs having the nucleotide sequence represented by SEQ ID NO: 937.

The probes having the nucleotide sequence represented by SEQ ID NOS: 16256, 16257, 16258, 16259, 16260, 16261, 16262, 16263, 16264, 16265, 16266 were selected from the ORFs having the nucleotide sequence represented by SEQ ID NO: 938.

The probes having the nucleotide sequence represented by SEQ ID NOS: 16267, 16268, 16269, 16270, 16271, 16272, 16273, 16274, 16275, 16276, 16277 were selected from the ORFs having the nucleotide sequence represented by SEQ ID NO: 939.

The probes having the nucleotide sequence represented by SEQ ID NOS: 16278, 16279, 16280, 16281, 16282, 16283, 16284, 16285, 16286, 16287, 16288 were selected from the ORFs having the nucleotide sequence represented by SEQ ID NO: 940.

The probes having the nucleotide sequence represented by SEQ ID NOS: 16289, 16290, 16291, 16292, 16293, 16294, 16295, 16296, 16297, 16298, 16299 were selected from the ORFs having the nucleotide sequence represented by SEQ ID NO: 941.

The probes having the nucleotide sequence represented by SEQ ID NOS: 16300, 16301, 16302, 16303, 16304, 16305, 16306, 16307, 16308, 16309, 16310 were selected from the ORFs having the nucleotide sequence represented by SEQ ID NO: 942.

The probes having the nucleotide sequence represented by SEQ ID NOS: 16311, 16312, 16313, 16314, 16315, 16316, 16317, 16318, 16319, 16320, 16321 were selected from the ORFs having the nucleotide sequence represented by SEQ ID NO: 943.

The probes having the nucleotide sequence represented by SEQ ID NOS: 16322, 16323, 16324, 16325, 16326, 16327, 16328, 16329, 16330, 16331, 16332 were selected from the ORFs having the nucleotide sequence represented by SEQ ID NO: 944.

The probes having the nucleotide sequence represented by SEQ ID NOS: 16333, 16334, 16335, 16336, 16337, 16338, 16339, 16340, 16341, 16342, 16343 were selected from the ORFs having the nucleotide sequence represented by SEQ ID NO: 945.

The probes having the nucleotide sequence represented by SEQ ID NOS: 16344, 16345, 16346, 16347, 16348, 16349, 16350, 16351, 16352, 16353, 16354 were selected from the ORFs having the nucleotide sequence represented by SEQ ID NO: 946.

The probes having the nucleotide sequence represented by SEQ ID NOS: 16355, 16356, 16357, 16358, 16359, 16360, 16361, 16362, 16363, 16364, 16365 were selected from the ORFs having the nucleotide sequence represented by SEQ ID NO: 947.

The probes having the nucleotide sequence represented by SEQ ID NOS: 16366, 16367, 16368, 16369, 16370, 16371, 16372, 16373, 16374, 16375, 16376 were selected from the ORFs having the nucleotide sequence represented by SEQ ID NO: 948.

The probes having the nucleotide sequence represented by SEQ ID NOS: 16377, 16378, 16379, 16380, 16381, 16382, 16383, 16384, 16385, 16386, 16387 were selected from the ORFs having the nucleotide sequence represented by SEQ ID NO: 949.

The probes having the nucleotide sequence represented by SEQ ID NOS: 16388, 16389, 16390, 16391, 16392, 16393, 16394, 16395, 16396, 16397, 16398 were selected from the ORFs having the nucleotide sequence represented by SEQ ID NO: 950.

The probes having the nucleotide sequence represented by SEQ ID NOS: 16399, 16400, 16401, 16402, 16403, 16404, 16405, 16406, 16407, 16408, 16409 were selected from the ORFs having the nucleotide sequence represented by SEQ ID NO: 951.

The probes having the nucleotide sequence represented by SEQ ID NOS: 16410, 16411, 16412, 16413, 16414, 16415, 16416, 16417, 16418, 16419, 16420 were selected from the ORFs having the nucleotide sequence represented by SEQ ID NO: 952.

The probes having the nucleotide sequence represented by SEQ ID NOS: 16421, 16422, 16423, 16424, 16425, 16426, 16427, 16428, 16429, 16430, 16431 were selected from the ORFs having the nucleotide sequence represented by SEQ ID NO: 953.

The probes having the nucleotide sequence represented by SEQ ID NOS: 16432, 16433, 16434, 16435, 16436, 16437, 16438, 16439, 16440, 16441, 16442 were selected from the ORFs having the nucleotide sequence represented by SEQ ID NO: 954.

The probes having the nucleotide sequence represented by SEQ ID NOS: 16443, 16444, 16445, 16446, 16447, 16448, 16449, 16450, 16451, 16452, 16453 were selected from the ORFs having the nucleotide sequence represented by SEQ ID NO: 955.

The probes having the nucleotide sequence represented by SEQ ID NOS: 16454, 16455, 16456, 16457, 16458, 16459, 16460, 16461, 16462, 16463, 16464 were selected from the ORFs having the nucleotide sequence represented by SEQ ID NO: 956.

The probes having the nucleotide sequence represented by SEQ ID NOS: 16465, 16466, 16467, 16468, 16469, 16470, 16471, 16472, 16473, 16474 were selected from the ORFs having the nucleotide sequence represented by SEQ ID NO: 957.

The probes having the nucleotide sequence represented by SEQ ID NOS: 16475, 16476, 16477, 16478, 16479, 16480, 16481, 16482, 16483, 16484, 16485 were selected from the ORFs having the nucleotide sequence represented by SEQ ID NO: 958.

The probes having the nucleotide sequence represented by SEQ ID NOS: 16486, 16487, 16488, 16489, 16490, 16491, 16492, 16493, 16494, 16495, 16496 were selected from the ORFs having the nucleotide sequence represented by SEQ ID NO: 959.

The probes having the nucleotide sequence represented by SEQ ID NOS: 16497, 16498, 16499, 16500, 16501, 16502, 16503, 16504, 16505, 16506, 16507 were selected from the ORFs having the nucleotide sequence represented by SEQ ID NO: 960.

The probes having the nucleotide sequence represented by SEQ ID NOS: 16508, 16509, 16510, 16511, 16512, 16513, 16514, 16515, 16516, 16517, 16518 were selected from the ORFs having the nucleotide sequence represented by SEQ ID NO: 961.

The probes having the nucleotide sequence represented by SEQ ID NOS: 16519, 16520, 16521, 16522, 16523, 16524, 16525, 16526, 16527, 16528, 16529 were selected from the ORFs having the nucleotide sequence represented by SEQ ID NO: 962.

The probes having the nucleotide sequence represented by SEQ ID NOS: 16530, 16531, 16532, 16533, 16534, 16535, 16536, 16537, 16538, 16539, 16540 were selected from the ORFs having the nucleotide sequence represented by SEQ ID NO: 963.

The probes having the nucleotide sequence represented by SEQ ID NOS: 16541, 16542, 16543, 16544, 16545, 16546, 16547, 16548, 16549, 16550, 16551 were selected from the ORFs having the nucleotide sequence represented by SEQ ID NO: 964.

The probes having the nucleotide sequence represented by SEQ ID NOS: 16552, 16553, 16554, 16555, 16556, 16557, 16558, 16559, 16560, 16561, 16562 were selected from the ORFs having the nucleotide sequence represented by SEQ ID NO: 965.

The probes having the nucleotide sequence represented by SEQ ID NOS: 16563, 16564, 16565, 16566, 16567, 16568, 16569, 16570, 16571, 16572, 16573 were selected from the ORFs having the nucleotide sequence represented by SEQ ID NO: 966.

The probes having the nucleotide sequence represented by SEQ ID NOS: 16574, 16575, 16576, 16577, 16578, 16579, 16580, 16581, 16582, 16583, 16584 were selected from the ORFs having the nucleotide sequence represented by SEQ ID NO: 967.

The probes having the nucleotide sequence represented by SEQ ID NOS: 16585, 16586, 16587, 16588, 16589, 16590, 16591, 16592, 16593, 16594, 16595 were selected from the ORFs having the nucleotide sequence represented by SEQ ID NO: 968.

The probes having the nucleotide sequence represented by SEQ ID NOS: 16596, 16597, 16598, 16599, 16600, 16601, 16602, 16603, 16604, 16605, 16606 were selected from the ORFs having the nucleotide sequence represented by SEQ ID NO: 969.

The probes having the nucleotide sequence represented by SEQ ID NOS: 16607, 16608, 16609, 16610, 16611, 16612, 16613, 16614, 16615, 16616, 16617 were selected from the ORFs having the nucleotide sequence represented by SEQ ID NO: 970.

The probes having the nucleotide sequence represented by SEQ ID NOS: 16618, 16619, 16620, 16621, 16622, 16623, 16624, 16625, 16626, 16627, 16628 were selected from the ORFs having the nucleotide sequence represented by SEQ ID NO: 971.

The probes having the nucleotide sequence represented by SEQ ID NOS: 16629, 16630, 16631, 16632, 16633, 16634, 16635, 16636, 16637, 16638, 16639 were selected from the ORFs having the nucleotide sequence represented by SEQ ID NO: 972.

The probes having the nucleotide sequence represented by SEQ ID NOS: 16640, 16641, 16642, 16643, 16644, 16645, 16646, 16647, 16648, 16649, 16650 were selected from the ORFs having the nucleotide sequence represented by SEQ ID NO: 973.

The probes having the nucleotide sequence represented by SEQ ID NOS: 16651, 16652, 16653, 16654, 16655, 16656, 16657, 16658, 16659, 16660, 16661 were selected from the ORFs having the nucleotide sequence represented by SEQ ID NO: 974.

The probes having the nucleotide sequence represented by SEQ ID NOS: 16662, 16663, 16664, 16665, 16666, 16667, 16668, 16669, 16670, 16671, 16672 were selected from the ORFs having the nucleotide sequence represented by SEQ ID NO: 975.

The probes having the nucleotide sequence represented by SEQ ID NOS: 16673, 16674, 16675, 16676, 16677, 16678, 16679, 16680, 16681, 16682, 16683 were selected from the ORFs having the nucleotide sequence represented by SEQ ID NO: 976.

The probes having the nucleotide sequence represented by SEQ ID NOS: 16684, 16685, 16686, 16687, 16688, 16689, 16690, 16691, 16692, 16693, 16694 were selected from the ORFs having the nucleotide sequence represented by SEQ ID NO: 977.

The probes having the nucleotide sequence represented by SEQ ID NOS: 16695, 16696, 16697, 16698, 16699, 16700, 16701, 16702, 16703, 16704, 16705 were selected from the ORFs having the nucleotide sequence represented by SEQ ID NO: 978.

The probes having the nucleotide sequence represented by SEQ ID NOS: 16706, 16707, 16708, 16709, 16710, 16711, 16712, 16713, 16714, 16715, 16716 were selected from the ORFs having the nucleotide sequence represented by SEQ ID NO: 979.

The probes having the nucleotide sequence represented by SEQ ID NOS: 16717, 16718, 16719, 16720, 16721, 16722, 16723, 16724, 16725, 16726, 16727 were selected from the ORFs having the nucleotide sequence represented by SEQ ID NO: 980.

The probes having the nucleotide sequence represented by SEQ ID NOS: 16728, 16729, 16730, 16731, 16732, 16733, 16734, 16735, 16736, 16737, 16738 were selected from the ORFs having the nucleotide sequence represented by SEQ ID NO: 981.

The probes having the nucleotide sequence represented by SEQ ID NOS: 16739, 16740, 16741, 16742, 16743, 16744, 16745, 16746, 16747, 16748, 16749 were selected from the ORFs having the nucleotide sequence represented by SEQ ID NO: 982.

The probes having the nucleotide sequence represented by SEQ ID NOS: 16750, 16751, 16752, 16753, 16754, 16755, 16756, 16757, 16758, 16759, 16760 were selected from the ORFs having the nucleotide sequence represented by SEQ ID NO: 983.

The probes having the nucleotide sequence represented by SEQ ID NOS: 16761, 16762, 16763, 16764, 16765, 16766, 16767, 16768, 16769, 16770, 16771 were selected from the ORFs having the nucleotide sequence represented by SEQ ID NO: 984.

The probes having the nucleotide sequence represented by SEQ ID NOS: 16772, 16773, 16774, 16775, 16776, 16777, 16778, 16779, 16780, 16781, 16782 were selected from the ORFs having the nucleotide sequence represented by SEQ ID NO: 985.

The probes having the nucleotide sequence represented by SEQ ID NOS: 16783, 16784, 16785, 16786, 16787, 16788, 16789, 16790, 16791, 16792, 16793 were selected from the ORFs having the nucleotide sequence represented by SEQ ID NO: 986.

The probes having the nucleotide sequence represented by SEQ ID NOS: 16794, 16795, 16796, 16797, 16798, 16799, 16800, 16801, 16802, 16803, 16804 were selected from the ORFs having the nucleotide sequence represented by SEQ ID NO: 987.

The probes having the nucleotide sequence represented by SEQ ID NOS: 16805, 16806, 16807, 16808, 16809, 16810, 16811, 16812, 16813, 16814, 16815 were selected from the ORFs having the nucleotide sequence represented by SEQ ID NO: 988.

The probes having the nucleotide sequence represented by SEQ ID NOS: 16816, 16817, 16818, 16819, 16820, 16821, 16822, 16823, 16824, 16825, 16826 were selected from the ORFs having the nucleotide sequence represented by SEQ ID NO: 989.

The probes having the nucleotide sequence represented by SEQ ID NOS: 16827, 16828, 16829, 16830, 16831, 16832, 16833, 16834, 16835, 16836, 16837 were selected from the ORFs having the nucleotide sequence represented by SEQ ID NO: 990.

The probes having the nucleotide sequence represented by SEQ ID NOS: 16838, 16839, 16840, 16841, 16842, 16843, 16844, 16845, 16846, 16847, 16848 were selected from the ORFs having the nucleotide sequence represented by SEQ ID NO: 991.

The probes having the nucleotide sequence represented by SEQ ID NOS: 16849, 16850, 16851, 16852, 16853, 16854, 16855, 16856, 16857, 16858, 16859 were selected from the ORFs having the nucleotide sequence represented by SEQ ID NO: 992.

The probes having the nucleotide sequence represented by SEQ ID NOS: 16860, 16861, 16862, 16863, 16864, 16865, 16866, 16867, 16868, 16869, 16870 were selected from the ORFs having the nucleotide sequence represented by SEQ ID NO: 993.

The probes having the nucleotide sequence represented by SEQ ID NOS: 16871, 16872, 16873, 16874, 16875, 16876, 16877, 16878, 16879, 16880, 16881 were selected from the ORFs having the nucleotide sequence represented by SEQ ID NO: 994.

The probes having the nucleotide sequence represented by SEQ ID NOS: 16882, 16883, 16884, 16885, 16886, 16887, 16888, 16889, 16890, 16891, 16892 were selected from the ORFs having the nucleotide sequence represented by SEQ ID NO: 995.

The probes having the nucleotide sequence represented by SEQ ID NOS: 16893, 16894, 16895, 16896, 16897, 16898, 16899, 16900, 16901, 16902, 16903 were selected from the ORFs having the nucleotide sequence represented by SEQ ID NO: 996.

The probes having the nucleotide sequence represented by SEQ ID NOS: 16904, 16905, 16906, 16907, 16908, 16909, 16910, 16911, 16912, 16913, 16914 were selected from the ORFs having the nucleotide sequence represented by SEQ ID NO: 997.

The probes having the nucleotide sequence represented by SEQ ID NOS: 16915, 16916, 16917, 16918, 16919, 16920, 16921, 16922, 16923, 16924, 16925 were selected from the ORFs having the nucleotide sequence represented by SEQ ID NO: 998.

The probes having the nucleotide sequence represented by SEQ ID NOS: 16926, 16927, 16928, 16929, 16930, 16931, 16932, 16933, 16934, 16935, 16936 were selected from the ORFs having the nucleotide sequence represented by SEQ ID NO: 999.

The probes having the nucleotide sequence represented by SEQ ID NOS: 16937, 16938, 16939, 16940, 16941, 16942, 16943, 16944, 16945, 16946, 16947 were selected from the ORFs having the nucleotide sequence represented by SEQ ID NO: 1000.

The probes having the nucleotide sequence represented by SEQ ID NOS: 16948, 16949, 16950, 16951, 16952, 16953, 16954, 16955, 16956, 16957, 16958 were selected from the ORFs having the nucleotide sequence represented by SEQ ID NO: 1001.

The probes having the nucleotide sequence represented by SEQ ID NOS: 16959, 16960, 16961, 16962, 16963, 16964, 16965, 16966, 16967, 16968, 16969 were selected from the ORFs having the nucleotide sequence represented by SEQ ID NO: 1002.

The probes having the nucleotide sequence represented by SEQ ID NOS: 16970, 16971, 16972, 16973, 16974, 16975, 16976, 16977, 16978, 16979, 16980 were selected from the ORFs having the nucleotide sequence represented by SEQ ID NO: 1003.

The probes having the nucleotide sequence represented by SEQ ID NOS: 16981, 16982, 16983, 16984, 16985, 16986, 16987, 16988, 16989, 16990, 16991 were selected from the ORFs having the nucleotide sequence represented by SEQ ID NO: 1004.

The probes having the nucleotide sequence represented by SEQ ID NOS: 16992, 16993, 16994, 16995, 16996, 16997, 16998, 16999, 17000, 17001, 17002 were selected from the ORFs having the nucleotide sequence represented by SEQ ID NO: 1005.

The probes having the nucleotide sequence represented by SEQ ID NOS: 17003, 17004, 17005, 17006, 17007, 17008, 17009, 17010, 17011, 17012, 17013 were selected from the ORFs having the nucleotide sequence represented by SEQ ID NO: 1006.

The probes having the nucleotide sequence represented by SEQ ID NOS: 17014, 17015, 17016, 17017, 17018, 17019, 17020, 17021, 17022, 17023, 17024 were selected from the ORFs having the nucleotide sequence represented by SEQ ID NO: 1007.

The probes having the nucleotide sequence represented by SEQ ID NOS: 17025, 17026, 17027, 17028, 17029, 17030, 17031, 17032, 17033, 17034, 17035 were selected from the ORFs having the nucleotide sequence represented by SEQ ID NO: 1008.

The probes having the nucleotide sequence represented by SEQ ID NOS: 17036, 17037, 17038, 17039, 17040, 17041, 17042, 17043, 17044, 17045, 17046 were selected from the ORFs having the nucleotide sequence represented by SEQ ID NO: 1009.

The probes having the nucleotide sequence represented by SEQ ID NOS: 17047, 17048, 17049, 17050, 17051, 17052, 17053, 17054, 17055, 17056, 17057 were selected from the ORFs having the nucleotide sequence represented by SEQ ID NO: 1010.

The probes having the nucleotide sequence represented by SEQ ID NOS: 17058, 17059, 17060, 17061, 17062, 17063, 17064, 17065, 17066, 17067, 17068 were selected from the ORFs having the nucleotide sequence represented by SEQ ID NO: 1011.

The probes having the nucleotide sequence represented by SEQ ID NOS: 17069, 17070, 17071, 17072, 17073, 17074, 17075, 17076, 17077, 17078, 17079 were selected from the ORFs having the nucleotide sequence represented by SEQ ID NO: 1012.

The probes having the nucleotide sequence represented by SEQ ID NOS: 17080, 17081, 17082, 17083, 17084, 17085, 17086, 17087, 17088, 17089, 17090 were selected from the ORFs having the nucleotide sequence represented by SEQ ID NO: 1013.

The probes having the nucleotide sequence represented by SEQ ID NOS: 17091, 17092, 17093, 17094, 17095, 17096, 17097, 17098, 17099, 17100, 17101 were selected from the ORFs having the nucleotide sequence represented by SEQ ID NO: 1014.

The probes having the nucleotide sequence represented by SEQ ID NOS: 17102, 17103, 17104, 17105, 17106, 17107, 17108, 17109, 17110, 17111, 17112 were selected from the ORFs having the nucleotide sequence represented by SEQ ID NO: 1015.

The probes having the nucleotide sequence represented by SEQ ID NOS: 17113, 17114, 17115, 17116, 17117, 17118, 17119, 17120, 17121, 17122, 17123 were selected from the ORFs having the nucleotide sequence represented by SEQ ID NO: 1016.

The probes having the nucleotide sequence represented by SEQ ID NOS: 17124, 17125, 17126, 17127, 17128, 17129, 17130, 17131, 17132, 17133, 17134 were selected from the ORFs having the nucleotide sequence represented by SEQ ID NO: 1017.

The probes having the nucleotide sequence represented by SEQ ID NOS: 17135, 17136, 17137, 17138, 17139, 17140, 17141, 17142, 17143, 17144, 17145 were selected from the ORFs having the nucleotide sequence represented by SEQ ID NO: 1018.

The probes having the nucleotide sequence represented by SEQ ID NOS: 17146, 17147, 17148, 17149, 17150, 17151, 17152, 17153, 17154, 17155, 17156 were selected from the ORFs having the nucleotide sequence represented by SEQ ID NO: 1019.

The probes having the nucleotide sequence represented by SEQ ID NOS: 17157, 17158, 17159, 17160, 17161, 17162, 17163, 17164, 17165, 17166, 17167 were selected from the ORFs having the nucleotide sequence represented by SEQ ID NO: 1020.

The probes having the nucleotide sequence represented by SEQ ID NOS: 17168, 17169, 17170, 17171, 17172, 17173, 17174, 17175, 17176, 17177, 17178 were selected from the ORFs having the nucleotide sequence represented by SEQ ID NO: 1021.

The probes having the nucleotide sequence represented by SEQ ID NOS: 17179, 17180, 17181, 17182, 17183, 17184, 17185, 17186, 17187, 17188, 17189 were selected from the ORFs having the nucleotide sequence represented by SEQ ID NO: 1022.

The probes having the nucleotide sequence represented by SEQ ID NOS: 17190, 17191, 1.7192, 17193, 17194, 17195, 17196, 17197, 17198, 17199, 17200 were selected from the ORFs having the nucleotide sequence represented by SEQ ID NO: 1023.

The probes having the nucleotide sequence represented by SEQ ID NOS: 17201, 17202, 17203, 17204, 17205, 17206, 17207, 17208, 17209, 17210, 17211 were selected from the ORFs having the nucleotide sequence represented by SEQ ID NO: 1024.

The probes having the nucleotide sequence represented by SEQ ID NOS: 17212, 17213, 17214, 17215, 17216, 17217, 17218, 17219, 17220, 17221, 17222 were selected from the ORFs having the nucleotide sequence represented by SEQ ID NO: 1025.

The probes having the nucleotide sequence represented by SEQ ID NOS: 17223, 17224, 17225, 17226, 17227, 17228, 17229, 17230, 17231, 17232, 17233 were selected from the ORFs having the nucleotide sequence represented by SEQ ID NO: 1026.

The probes having the nucleotide sequence represented by SEQ ID NOS: 17234, 17235, 17236, 17237, 17238, 17239, 17240, 17241, 17242, 17243, 17244 were selected from the ORFs having the nucleotide sequence represented by SEQ ID NO: 1027.

The probes having the nucleotide sequence represented by SEQ ID NOS: 17245, 17246, 17247, 17248, 17249, 17250, 17251, 17252, 17253, 17254, 17255 were selected from the ORFs having the nucleotide sequence represented by SEQ ID NO: 1028.

The probes having the nucleotide sequence represented by SEQ ID NOS: 17256, 17257, 17258, 17259, 17260, 17261, 17262, 17263, 17264, 17265, 17266 were selected from the ORFs having the nucleotide sequence represented by SEQ ID NO: 1029.

The probes having the nucleotide sequence represented by SEQ ID NOS: 17267, 17268, 17269, 17270, 17271, 17272, 17273, 17274, 17275, 17276, 17277 were selected from the ORFs having the nucleotide sequence represented by SEQ ID NO: 1030.

The probes having the nucleotide sequence represented by SEQ ID NOS: 17278, 17279, 17280, 17281, 17282, 17283, 17284, 17285, 17286, 17287, 17288 were selected from the ORFs having the nucleotide sequence represented by SEQ ID NO: 1031.

The probes having the nucleotide sequence represented by SEQ ID NOS: 17289, 17290, 17291, 17292, 17293, 17294, 17295, 17296, 17297, 17298, 17299 were selected from the ORFs having the nucleotide sequence represented by SEQ ID NO: 1032.

The probes having the nucleotide sequence represented by SEQ ID NOS: 17300, 17301, 17302, 17303, 17304, 17305, 17306, 17307, 17308, 17309, 17310 were selected from the ORFs having the nucleotide sequence represented by SEQ ID NO: 1033.

The probes having the nucleotide sequence represented by SEQ ID NOS: 17311, 17312, 17313, 17314, 17315, 17316, 17317, 17318, 17319, 17320, 17321 were selected from the ORFs having the nucleotide sequence represented by SEQ ID NO: 1034.

The probes having the nucleotide sequence represented by SEQ ID NOS: 17322, 17323, 17324, 17325, 17326, 17327, 17328, 17329, 17330, 17331, 17332 were selected from the ORFs having the nucleotide sequence represented by SEQ ID NO: 1035.

The probes having the nucleotide sequence represented by SEQ ID NOS: 17333, 17334, 17335, 17336, 17337, 17338, 17339, 17340, 17341, 17342, 17343 were selected from the ORFs having the nucleotide sequence represented by SEQ ID NO: 1036.

The probes having the nucleotide sequence represented by SEQ ID NOS: 17344, 17345, 17346, 17347, 17348, 17349, 17350, 17351, 17352, 17353, 17354 were selected from the ORFs having the nucleotide sequence represented by SEQ ID NO: 1037.

The probes having the nucleotide sequence represented by SEQ ID NOS: 17355, 17356, 17357, 17358, 17359, 17360, 17361, 17362, 17363, 17364, 17365 were selected from the ORFs having the nucleotide sequence represented by SEQ ID NO: 1038.

The probes having the nucleotide sequence represented by SEQ ID NOS: 17366, 17367, 17368, 17369, 17370, 17371, 17372, 17373, 17374, 17375, 17376 were selected from the ORFs having the nucleotide sequence represented by SEQ ID NO: 1039.

The probes having the nucleotide sequence represented by SEQ ID NOS: 17377, 17378, 17379, 17380, 17381, 17382, 17383, 17384, 17385, 17386, 17387 were selected from the ORFs having the nucleotide sequence represented by SEQ ID NO: 1040.

The probes having the nucleotide sequence represented by SEQ ID NOS: 17388, 17389, 17390, 17391, 17392, 17393, 17394, 17395, 17396, 17397, 17398 were selected from the ORFs having the nucleotide sequence represented by SEQ ID NO: 1041.

The probes having the nucleotide sequence represented by SEQ ID NOS: 17399, 17400, 17401, 17402, 17403, 17404, 17405, 17406, 17407, 17408, 17409 were selected from the ORFs having the nucleotide sequence represented by SEQ ID NO: 1042.

The probes having the nucleotide sequence represented by SEQ ID NOS: 17410, 17411, 17412, 17413, 17414, 17415, 17416, 17417, 17418, 17419, 17420 were selected from the ORFs having the nucleotide sequence represented by SEQ ID NO: 1043.

The probes having the nucleotide sequence represented by SEQ ID NOS: 17421, 17422, 17423, 17424, 17425, 17426, 17427, 17428, 17429, 17430, 17431 were selected from the ORFs having the nucleotide sequence represented by SEQ ID NO: 1044.

The probes having the nucleotide sequence represented by SEQ ID NOS: 17432, 17433, 17434, 17435, 17436, 17437, 17438, 17439, 17440, 17441, 17442 were selected from the ORFs having the nucleotide sequence represented by SEQ ID NO: 1045.

The probes having the nucleotide sequence represented by SEQ ID NOS: 17443, 17444, 17445, 17446, 17447, 17448, 17449, 17450, 17451, 17452, 17453 were selected from the ORFs having the nucleotide sequence represented by SEQ ID NO: 1046.

The probes having the nucleotide sequence represented by SEQ ID NOS: 17454, 17455, 17456, 17457, 17458, 17459, 17460, 17461, 17462, 17463, 17464 were selected from the ORFs having the nucleotide sequence represented by SEQ ID NO: 1047.

The probes having the nucleotide sequence represented by SEQ ID NOS: 17465, 17466, 17467, 17468, 17469, 17470, 17471, 17472, 17473, 17474, 17475 were selected from the ORFs having the nucleotide sequence represented by SEQ ID NO: 1048.

The probes having the nucleotide sequence represented by SEQ ID NOS: 17476, 17477, 17478, 17479, 17480, 17481, 17482, 17483, 17484, 17485, 17486 were selected from the ORFs having the nucleotide sequence represented by SEQ ID NO: 1049.

The probes having the nucleotide sequence represented by SEQ ID NOS: 17487, 17488, 17489, 17490, 17491, 17492, 17493, 17494, 17495, 17496, 17497 were selected from the ORFs having the nucleotide sequence represented by SEQ ID NO: 1050.

The probes having the nucleotide sequence represented by SEQ ID NOS: 17498, 17499, 17500, 17501, 17502, 17503, 17504, 17505, 17506, 17507, 17508 were selected from the ORFs having the nucleotide sequence represented by SEQ ID NO: 1051.

The probes having the nucleotide sequence represented by SEQ ID NOS: 17509, 17510, 17511, 17512, 17513, 17514, 17515, 17516, 17517, 17518, 17519 were selected from the ORFs having the nucleotide sequence represented by SEQ ID NO: 1052.

The probes having the nucleotide sequence represented by SEQ ID NOS: 17520, 17521, 17522, 17523, 17524, 17525, 17526, 17527, 17528, 17529, 17530 were selected from the ORFs having the nucleotide sequence represented by SEQ ID NO: 1053.

The probes having the nucleotide sequence represented by SEQ ID NOS: 17531, 17532, 17533, 17534, 17535, 17536, 17537, 17538, 17539, 17540, 17541 were selected from the ORFs having the nucleotide sequence represented by SEQ ID NO: 1054.

The probes having the nucleotide sequence represented by SEQ ID NOS: 17542, 17543, 17544, 17545, 17546, 17547, 17548, 17549, 17550, 17551, 17552 were selected from the ORFs having the nucleotide sequence represented by SEQ ID NO: 1055.

The probes having the nucleotide sequence represented by SEQ ID NOS: 17553, 17554, 17555, 17556, 17557, 17558, 17559, 17560, 17561, 17562, 17563 were selected from the ORFs having the nucleotide sequence represented by SEQ ID NO: 1056.

The probes having the nucleotide sequence represented by SEQ ID NOS: 17564, 17565, 17566, 17567, 17568, 17569, 17570, 17571, 17572, 17573, 17574 were selected from the ORFs having the nucleotide sequence represented by SEQ ID NO: 1057.

The probes having the nucleotide sequence represented by SEQ ID NOS: 17575, 17576, 17577, 17578, 17579, 17580, 17581, 17582, 17583, 17584, 17585 were selected from the ORFs having the nucleotide sequence represented by SEQ ID NO: 1058.

The probes having the nucleotide sequence represented by SEQ ID NOS: 17586, 17587, 17588, 17589, 17590, 17591, 17592, 17593, 17594, 17595, 17596 were selected from the ORFs having the nucleotide sequence represented by SEQ ID NO: 1059.

The probes having the nucleotide sequence represented by SEQ ID NOS: 17597, 17598, 17599, 17600, 17601, 17602, 17603, 17604, 17605, 17606, 17607 were selected from the ORFs having the nucleotide sequence represented by SEQ ID NO: 1060.

The probes having the nucleotide sequence represented by SEQ ID NOS: 17608, 17609, 17610, 17611, 17612, 17613, 17614, 17615, 17616, 17617, 17618 were selected from the ORFs having the nucleotide sequence represented by SEQ ID NO: 1061.

The probes having the nucleotide sequence represented by SEQ ID NOS: 17619, 17620, 17621, 17622, 17623, 17624, 17625, 17626, 17627, 17628, 17629 were selected from the ORFs having the nucleotide sequence represented by SEQ ID NO: 1062.

The probes having the nucleotide sequence represented by SEQ ID NOS: 17630, 17631, 17632, 17633, 17634, 17635, 17636, 17637, 17638, 17639, 17640 were selected from the ORFs having the nucleotide sequence represented by SEQ ID NO: 1063.

The probes having the nucleotide sequence represented by SEQ ID NOS: 17641, 17642, 17643, 17644, 17645, 17646, 17647, 17648, 17649, 17650, 17651 were selected from the ORFs having the nucleotide sequence represented by SEQ ID NO: 1064.

The probes having the nucleotide sequence represented by SEQ ID NOS: 17652, 17653, 17654, 17655, 17656, 17657, 17658, 17659, 17660, 17661, 17662 were selected from the ORFs having the nucleotide sequence represented by SEQ ID NO: 1065.

The probes having the nucleotide sequence represented by SEQ ID NOS: 17663, 17664, 17665, 17666, 17667, 17668, 17669, 17670, 17671, 17672, 17673 were selected from the ORFs having the nucleotide sequence represented by SEQ ID NO: 1066.

The probes having the nucleotide sequence represented by SEQ ID NOS: 17674, 17675, 17676, 17677, 17678, 17679, 17680, 17681, 17682, 17683, 17684 were selected from the ORFs having the nucleotide sequence represented by SEQ ID NO: 1067.

The probes having the nucleotide sequence represented by SEQ ID NOS: 17685, 17686, 17687, 17688, 17689, 17690, 17691, 17692, 17693, 17694, 17695 were selected from the ORFs having the nucleotide sequence represented by SEQ ID NO: 1068.

The probes having the nucleotide sequence represented by SEQ ID NOS: 17696, 17697, 17698, 17699, 17700, 17701, 17702, 17703, 17704, 17705, 17706 were selected from the ORFs having the nucleotide sequence represented by SEQ ID NO: 1069.

The probes having the nucleotide sequence represented by SEQ ID NOS: 17707, 17708, 17709, 17710, 17711, 17712, 17713, 17714, 17715, 17716, 17717 were selected from the ORFs having the nucleotide sequence represented by SEQ ID NO: 1070.

The probes having the nucleotide sequence represented by SEQ ID NOS: 17718, 17719, 17720, 17721, 17722, 17723, 17724, 17725, 17726, 17727, 17728 were selected from the ORFs having the nucleotide sequence represented by SEQ ID NO: 1071.

The probes having the nucleotide sequence represented by SEQ ID NOS: 17729, 17730, 17731, 17732, 17733, 17734, 17735, 17736, 17737, 17738, 17739 were selected from the ORFs having the nucleotide sequence represented by SEQ ID NO: 1072.

The probes having the nucleotide sequence represented by SEQ ID NOS: 17740, 17741, 17742, 17743, 17744, 17745, 17746, 17747, 17748, 17749, 17750 were selected from the ORFs having the nucleotide sequence represented by SEQ ID NO: 1073.

The probes having the nucleotide sequence represented by SEQ ID NOS: 17751, 17752, 17753, 17754, 17755, 17756, 17757, 17758, 17759, 17760, 17761 were selected from the ORFs having the nucleotide sequence represented by SEQ ID NO: 1074.

The probes having the nucleotide sequence represented by SEQ ID NOS: 17762, 17763, 17764, 17765, 17766, 17767, 17768, 17769, 17770, 17771, 17772 were selected from the ORFs having the nucleotide sequence represented by SEQ ID NO: 1075.

The probes having the nucleotide sequence represented by SEQ ID NOS: 17773, 17774, 17775, 17776, 17777, 17778, 17779, 17780, 17781, 17782, 17783 were selected from the ORFs having the nucleotide sequence represented by SEQ ID NO: 1076.

The probes having the nucleotide sequence represented by SEQ ID NOS: 17784, 17785, 17786, 17787, 17788, 17789, 17790, 17791, 17792, 17793, 17794 were selected from the ORFs having the nucleotide sequence represented by SEQ ID NO: 1077.

The probes having the nucleotide sequence represented by SEQ ID NOS: 17795, 17796, 17797, 17798, 17799, 17800, 17801, 17802, 17803, 17804, 17805 were selected,from the ORFs having the nucleotide sequence represented by SEQ ID NO: 1078.

The probes having the nucleotide sequence represented by SEQ ID NOS: 17806, 17807, 17808, 17809, 17810, 17811, 17812, 17813, 17814, 17815, 17816 were selected from the ORFs having the nucleotide sequence represented by SEQ ID NO: 1079.

The probes having the nucleotide sequence represented by SEQ ID NOS: 17817, 17818, 17819, 17820, 17821, 17822, 17823, 17824, 17825, 17826, 17827 were selected from the ORFs having the nucleotide sequence represented by SEQ ID NO: 1080.

The probes having the nucleotide sequence represented by SEQ ID NOS: 17828, 17829, 17830, 17831, 17832, 17833, 17834, 17835, 17836, 17837, 17838 were selected from the ORFs having the nucleotide sequence represented by SEQ ID NO: 1081.

The probes having the nucleotide sequence represented by SEQ ID NOS: 17839, 17840, 17841, 17842, 17843, 17844, 17845, 17846, 17847, 17848, 17849 were selected from the ORFs having the nucleotide sequence represented by SEQ ID NO: 1082.

The probes having the nucleotide sequence represented by SEQ ID NOS: 17850, 17851, 17852, 17853, 17854, 17855, 17856, 17857, 17858, 17859, 17860 were selected from the ORFs having the nucleotide sequence represented by SEQ ID NO: 1083.

The probes having the nucleotide sequence represented by SEQ ID NOS: 17861, 17862, 17863, 17864, 17865, 17866, 17867, 17868, 17869, 17870, 17871 were selected from the ORFs having the nucleotide sequence represented by SEQ ID NO: 1084.

The probes having the nucleotide sequence represented by SEQ ID NOS: 17872, 17873, 17874, 17875, 17876, 17877, 17878, 17879, 17880, 17881, 17882 were selected from the ORFs having the nucleotide sequence represented by SEQ ID NO: 1085.

The probes having the nucleotide sequence represented by SEQ ID NOS: 17883, 17884, 17885, 17886, 17887, 17888, 17889, 17890, 17891, 17892, 17893 were selected from the ORFs having the nucleotide sequence represented by SEQ ID NO: 1086.

The probes having the nucleotide sequence represented by SEQ ID NOS: 17894, 17895, 17896, 17897, 17898, 17899, 17900, 17901, 17902, 17903, 17904 were selected from the ORFs having the nucleotide sequence represented by SEQ ID NO: 1087.

The probes having the nucleotide sequence represented by SEQ ID NOS: 17905, 17906, 17907, 17908, 17909, 17910, 17911, 17912, 17913, 17914, 17915 were selected from the ORFs having the nucleotide sequence represented by SEQ ID NO: 1088.

The probes having the nucleotide sequence represented by SEQ ID NOS: 17916, 17917, 17918, 17919, 17920, 17921, 17922, 17923, 17924, 17925, 17926 were selected from the ORFs having the nucleotide sequence represented by SEQ ID NO: 1089.

The probes having the nucleotide sequence represented by SEQ ID NOS: 17927, 17928, 17929, 17930, 17931, 17932, 17933, 17934, 17935, 17936, 17937 were selected from the ORFs having the nucleotide sequence represented by SEQ ID NO: 1090.

The probes having the nucleotide sequence represented by SEQ ID NOS: 17938, 17939, 17940, 17941, 17942, 17943, 17944, 17945, 17946, 17947, 17948 were selected from the ORFs having the nucleotide sequence represented by SEQ ID NO: 1091.

The probes having the nucleotide sequence represented by SEQ ID NOS: 17949, 17950, 17951, 17952, 17953, 17954, 17955, 17956, 17957, 17958, 17959 were selected from the ORFs having the nucleotide sequence represented by SEQ ID NO: 1092.

The probes having the nucleotide sequence represented by SEQ ID NOS: 17960, 17961, 17962, 17963, 17964, 17965, 17966, 17967, 17968, 17969, 17970 were selected from the ORFs having the nucleotide sequence represented by SEQ ID NO: 1093.

The probes having the nucleotide sequence represented by SEQ ID NOS: 17971, 17972, 17973, 17974, 17975, 17976, 17977, 17978, 17979, 17980, 17981 were selected from the ORFs having the nucleotide sequence represented by SEQ ID NO: 1094.

The probes having the nucleotide sequence represented by SEQ ID NOS: 17982, 17983, 17984, 17985, 17986, 17987, 17988, 17989, 17990, 17991, 17992 were selected from the ORFs having the nucleotide sequence represented by SEQ ID NO: 1095.

The probes having the nucleotide sequence represented by SEQ ID NOS: 17993, 17994, 17995, 17996, 17997, 17998, 17999, 18000, 18001, 18002, 18003 were selected from the ORFs having the nucleotide sequence represented by SEQ ID NO: 1096.

The probes having the nucleotide sequence represented by SEQ ID NOS: 18004, 18005, 18006, 18007, 18008, 18009, 18010, 18011, 18012, 18013, 18014 were selected from the ORFs having the nucleotide sequence represented by SEQ ID NO: 1097.

The probes having the nucleotide sequence represented by SEQ ID NOS: 18015, 18016, 18017, 18018, 18019, 18020, 18021, 18022, 18023, 18024, 18025 were selected from the ORFs having the nucleotide sequence represented by SEQ ID NO: 1098.

The probes having the nucleotide sequence represented by SEQ ID NOS: 18026, 18027, 18028, 18029, 18030, 18031, 18032, 18033, 18034, 18035, 18036 were selected from the ORFs having the nucleotide sequence represented by SEQ ID NO: 1099.

The probes having the nucleotide sequence represented by SEQ ID NOS: 18037, 18038, 18039, 18040, 18041, 18042, 18043, 18044, 18045, 18046, 18047 were selected from the ORFs having the nucleotide sequence represented by SEQ ID NO: 1100.

The probes having the nucleotide sequence represented by SEQ ID NOS: 18048, 18049, 18050, 18051, 18052, 18053, 18054, 18055, 18056, 18057, 18058 were selected from the ORFs having the nucleotide sequence represented by SEQ ID NO: 1101.

The probes having the nucleotide sequence represented by SEQ ID NOS: 18059, 18060, 18061, 18062, 18063, 18064, 18065, 18066, 18067, 18068, 18069 were selected from the ORFs having the nucleotide sequence represented by SEQ ID NO: 1102.

The probes having the nucleotide sequence represented by SEQ ID NOS: 18070, 18071, 18072, 18073, 18074, 18075, 18076, 18077, 18078, 18079, 18080 were selected from the ORFs having the nucleotide sequence represented by SEQ ID NO: 1103.

The probes having the nucleotide sequence represented by SEQ ID NOS: 18081, 18082, 18083, 18084, 18085, 18086, 18087, 18088, 18089, 18090, 18091 were selected from the ORFs having the nucleotide sequence represented by SEQ ID NO: 1104.

The probes having the nucleotide sequence represented by SEQ ID NOS: 18092, 18093, 18094, 18095, 18096, 18097, 18098, 18099, 18100, 18101, 18102 were selected from the ORFs having the nucleotide sequence represented by SEQ ID NO: 1105.

The probes having the nucleotide sequence represented by SEQ ID NOS: 18103, 18104, 18105, 18106, 18107, 18108, 18109, 18110, 18111, 18112, 18113 were selected from the ORFs having the nucleotide sequence represented by SEQ ID NO: 1106.

The probes having the nucleotide sequence represented by SEQ ID NOS: 18114, 18115, 18116, 18117, 18118, 18119, 18120, 18121, 18122, 18123, 18124 were selected from the ORFs having the nucleotide sequence represented by SEQ ID NO: 1107.

The probes having the nucleotide sequence represented by SEQ ID NOS: 18125, 18126, 18127, 18128, 18129, 18130, 18131, 18132, 18133, 18134, 18135 were selected from the ORFs having the nucleotide sequence represented by SEQ ID NO: 1108.

The probes having the nucleotide sequence represented by SEQ ID NOS: 18136, 18137, 18138, 18139, 18140, 18141, 18142, 18143, 18144, 18145, 18146 were selected from the ORFs having the nucleotide sequence represented by SEQ ID NO: 1109.

The probes having the nucleotide sequence represented by SEQ ID NOS: 18147, 18148, 18149, 18150, 18151, 18152, 18153, 18154, 18155, 18156, 18157 were selected from the ORFs having the nucleotide sequence represented by SEQ ID NO: 1110.

The probes having the nucleotide sequence represented by SEQ ID NOS: 18158, 18159, 18160, 18161, 18162, 18163, 18164, 18165, 18166, 18167, 18168 were selected from the ORFs having the nucleotide sequence represented by SEQ ID NO: 1111.

The probes having the nucleotide sequence represented by SEQ ID NOS: 18169, 1.8170, 18171, 18172, 18173, 18174, 18175, 18176, 18177, 18178, 18179 were selected from the ORFs having the nucleotide sequence represented by SEQ ID NO: 1112.

The probes having the nucleotide sequence represented by SEQ ID NOS: 18180, 18181, 18182, 18183, 18184, 18185, 18186, 18187, 18188, 18189, 18190 were selected from the ORFs having the nucleotide sequence represented by SEQ ID NO: 1113.

The probes having the nucleotide sequence represented by SEQ ID NOS: 18191, 18192, 18193, 18194, 18195, 18196, 18197, 18198, 18199, 18200, 18201 were selected from the ORFs having the nucleotide sequence represented by SEQ ID NO: 1114.

The probes having the nucleotide sequence represented by SEQ ID NOS: 18202, 18203, 18204, 18205, 18206, 18207, 18208, 18209, 18210, 18211, 18212 were selected from the ORFs having the nucleotide sequence represented by SEQ ID NO: 1115.

The probes having the nucleotide sequence represented by SEQ ID NOS: 18213, 18214, 18215, 18216, 18217, 18218, 18219, 18220, 18221, 18222, 18223 were selected from the ORFs having the nucleotide sequence represented by SEQ ID NO: 1116.

The probes having the nucleotide sequence represented by SEQ ID NOS: 18224, 18225, 18226, 18227, 18228, 18229, 18230, 18231, 18232, 18233, 18234 were selected from the ORFs having the nucleotide sequence represented by SEQ ID NO: 1117.

The probes having the nucleotide sequence represented by SEQ ID NOS: 18235, 18236, 18237, 18238, 18239, 18240, 18241, 18242, 18243, 18244, 18245 were selected from the ORFs having the nucleotide sequence represented by SEQ ID NO: 1118.

The probes having the nucleotide sequence represented by SEQ ID NOS: 18246, 18247, 18248, 18249, 18250, 18251, 18252, 18253, 18254, 18255, 18256 were selected from the ORFs having the nucleotide sequence represented by SEQ ID NO: 1119.

The probes having the nucleotide sequence represented by SEQ ID NOS: 18257, 18258, 18259, 18260, 18261, 18262, 18263, 18264, 18265, 18266, 18267 were selected from the ORFs having the nucleotide sequence represented by SEQ ID NO: 1120.

The probes having the nucleotide sequence represented by SEQ ID NOS: 18268, 18269, 18270, 18271, 18272, 18273, 18274, 18275, 18276, 18277, 18278 were selected from the ORFs having the nucleotide sequence represented by SEQ ID NO: 1121.

The probes having the nucleotide sequence represented by SEQ ID NOS: 18279, 18280, 18281, 18282, 18283, 18284, 18285, 18286, 18287, 18288, 18289 were selected from the ORFs having the nucleotide sequence represented by SEQ ID NO: 1122.

The probes having the nucleotide sequence represented by SEQ ID NOS: 18290, 18291, 18292, 18293, 18294, 18295, 18296, 18297, 18298, 18299, 18300 were selected from the ORFs having the nucleotide sequence represented by SEQ ID NO: 1123.

The probes having the nucleotide sequence represented by SEQ ID NOS: 18301, 18302, 18303, 18304, 18305, 18306, 18307, 18308, 18309, 18310, 18311 were selected from the ORFs having the nucleotide sequence represented by SEQ ID NO: 1124.

The probes having the nucleotide sequence represented by SEQ ID NOS: 18312, 18313, 18314, 18315, 18316, 18317, 18318, 18319, 18320, 18321, 18322 were selected from the ORFs having the nucleotide sequence represented by SEQ ID NO: 1125.

The probes having the nucleotide sequence represented by SEQ ID NOS: 18323, 18324, 18325, 18326, 18327, 18328, 18329, 18330, 18331, 18332, 18333, 18334, 18335, 18336, 18337, 18338, 18339, 18340, 18341, 18342, 18343, 18344 were selected from the ORFs having the nucleotide sequence represented by SEQ ID NO: 1126.

The probes having the nucleotide sequence represented by SEQ ID NOS: 18345, 18346, 18347, 18348, 18349, 18350, 18351, 18352, 18353, 18354, 18355 were selected from the ORFs having the nucleotide sequence represented by SEQ ID NO: 1127.

The probes having the nucleotide sequence represented by SEQ ID NOS: 18356, 18357, 18358, 18359, 18360, 18361, 18362, 18363, 18364, 18365, 18366 were selected from the ORFs having the nucleotide sequence represented by SEQ ID NO: 1128.

The probes having the nucleotide sequence represented by SEQ ID NOS: 18367, 18368, 18369, 18370, 18371, 18372, 18373, 18374, 18375, 18376, 18377 were selected from the ORFs having the nucleotide sequence represented by SEQ ID NO: 1129.

The probes having the nucleotide sequence represented by SEQ ID NOS: 18378, 18379, 18380, 18381, 18382, 18383, 18384, 18385, 18386, 18387, 18388 were selected from the ORFs having the nucleotide sequence represented by SEQ ID NO: 1130.

The probes having the nucleotide sequence represented by SEQ ID NOS: 18389, 18390, 18391, 18392, 18393, 18394, 18395, 18396, 18397, 18398, 18399, 18400, 18401, 18402, 18403, 18404, 18405, 18406, 18407, 18408, 18409, 18410 were selected from the ORFs having the nucleotide sequence represented by SEQ ID NO: 1131.

The probes having the nucleotide sequence represented by SEQ ID NOS: 18411, 18412, 18413, 18414, 18415, 18416, 18417, 18418, 18419, 18420, 18421 were selected from the ORFs having the nucleotide sequence represented by SEQ ID NO: 1132.

The probes having the nucleotide sequence represented by SEQ ID NOS: 18422, 18423, 18424, 18425, 18426, 18427, 18428, 18429, 18430, 18431, 18432 were selected from the ORFs having the nucleotide sequence represented by SEQ ID NO: 1133.

The probes having the nucleotide sequence represented by SEQ ID NOS: 18433, 18434, 18435, 18436, 18437, 18438, 18439, 18440, 18441, 18442, 18443 were selected from the ORFs having the nucleotide sequence represented by SEQ ID NO: 1134.

The probes having the nucleotide sequence represented by SEQ ID NOS: 18444, 18445, 18446, 18447, 18448, 18449, 18450, 18451, 18452, 18453, 18454 were selected from the ORFs having the nucleotide sequence represented by SEQ ID NO: 1135.

The probes having the nucleotide sequence represented by SEQ ID NOS: 18455, 18456, 18457, 18458, 18459, 18460, 18461, 18462, 18463, 18464, 18465 were selected from the ORFs having the nucleotide sequence represented by SEQ ID NO: 1136.

The probes having the nucleotide sequence represented by SEQ ID NOS: 18466, 18467, 18468, 18469, 18470, 18471, 18472, 18473, 18474, 18475, 18476 were selected from the ORFs having the nucleotide sequence represented by SEQ ID NO: 1137.

The probes having the nucleotide sequence represented by SEQ ID NOS: 18477, 18478, 18479, 18480, 18481, 18482, 18483, 18484, 18485, 18486, 18487 were selected from the ORFs having the nucleotide sequence represented by SEQ ID NO: 1138.

The probes having the nucleotide sequence represented by SEQ ID NOS: 18488, 18489, 18490, 18491, 18492, 18493, 18494, 18495, 18496, 18497, 18498 were selected from the ORFs having the nucleotide sequence represented by SEQ ID NO: 1139.

The probes having the nucleotide sequence represented by SEQ ID NOS: 18499, 18500, 18501, 18502, 18503, 18504, 18505, 18506, 18507, 18508, 18509 were selected from the ORFs having the nucleotide sequence represented by SEQ ID NO: 1140.

The probes having the nucleotide sequence represented by SEQ ID NOS: 18510, 18511, 18512, 18513, 18514, 18515, 18516, 18517, 18518, 18519, 18520 were selected from the ORFs having the nucleotide sequence represented by SEQ ID NO: 1141.

The probes having the nucleotide sequence represented by SEQ ID NOS: 18521, 18522, 18523, 18524, 18525, 18526, 18527, 18528, 18529, 18530, 18531 were selected from the ORFs having the nucleotide sequence represented by SEQ ID NO: 1142.

The probes having the nucleotide sequence represented by SEQ ID NOS: 18532, 18533, 18534, 18535, 18536, 18537, 18538, 18539, 18540, 18541, 18542 were selected from the ORFs having the nucleotide sequence represented by SEQ ID NO: 1143.

The probes having the nucleotide sequence represented by SEQ ID NOS: 18543, 18544, 18545, 18546, 18547, 18548, 18549, 18550, 18551, 18552, 18553 were selected from the ORFs having the nucleotide sequence represented by SEQ ID NO: 1144.

The probes having the nucleotide sequence represented by SEQ ID NOS: 18554, 18555, 18556, 18557, 18558, 18559, 18560, 18561, 18562, 18563, 18564 were selected from the ORFs having the nucleotide sequence represented by SEQ ID NO: 1145.

The probes having the nucleotide sequence represented by SEQ ID NOS: 18565, 18566, 18567, 18568, 18569, 18570, 18571, 18572, 18573, 18574, 18575 were selected from the ORFs having the nucleotide sequence represented by SEQ ID NO: 1146.

The probes having the nucleotide sequence represented by SEQ ID NOS: 18576, 18577, 18578, 18579, 18580, 18581, 18582, 18583, 18584, 18585, 18586 were selected from the ORFs having the nucleotide sequence represented by SEQ ID NO: 1147.

The probes having the nucleotide sequence represented by SEQ ID NOS: 18587, 18588, 18589, 18590, 18591, 18592, 18593, 18594, 18595, 18596, 18597 were selected from the ORFs having the nucleotide sequence represented by SEQ ID NO: 1148.

The probes having the nucleotide sequence represented by SEQ ID NOS: 18598, 18599, 18600, 18601, 18602, 18603, 18604, 18605, 18606, 18607, 18608 were selected from the ORFs having the nucleotide sequence represented by SEQ ID NO: 1149.

The probes having the nucleotide sequence represented by SEQ ID NOS: 18609, 18610, 18611, 18612, 18613, 18614, 18615, 18616, 18617, 18618, 18619 were selected from the ORFs having the nucleotide sequence represented by SEQ ID NO: 1150.

The probes having the nucleotide sequence represented by SEQ ID NOS: 18620, 18621, 18622, 18623, 18624, 18625, 18626, 18627, 18628, 18629, 18630 were selected from the ORFs having the nucleotide sequence represented by SEQ ID NO: 1151.

The probes having the nucleotide sequence represented by SEQ ID NOS: 18631, 18632, 18633, 18634, 18635, 18636, 18637, 18638, 18639, 18640, 18641 were selected from the ORFs having the nucleotide sequence represented by SEQ ID NO: 1152.

The probes having the nucleotide sequence represented by SEQ ID NOS: 18642, 18643, 18644, 18645, 18646, 18647, 18648, 18649, 18650, 18651, 18652 were selected from the ORFs having the nucleotide sequence represented by SEQ ID NO: 1153.

The probes having the nucleotide sequence represented by SEQ ID NOS: 18653, 18654, 18655, 18656, 18657, 18658, 18659, 18660, 18661, 18662, 18663 were selected from the ORFs having the nucleotide sequence represented by SEQ ID NO: 1154.

The probes having the nucleotide sequence represented by SEQ ID NOS: 18664, 18665, 18666, 18667, 18668, 18669, 18670, 18671, 18672, 18673, 18674 were selected from the ORFs having the nucleotide sequence represented by SEQ ID NO: 1155.

The probes having the nucleotide sequence represented by SEQ ID NOS: 18675, 18676, 18677, 18678, 18679, 18680, 18681, 18682, 18683, 18684, 18685 were selected from the ORFs having the nucleotide sequence represented by SEQ ID NO: 1156.

The probes having the nucleotide sequence represented by SEQ ID NOS: 18686, 18687, 18688, 18689, 18690, 18691, 18692, 18693, 18694, 18695, 18696 were selected from the ORFs having the nucleotide sequence represented by SEQ ID NO: 1157.

The probes having the nucleotide sequence represented by SEQ ID NOS: 18697, 18698, 18699, 18700, 18701, 18702, 18703, 18704, 18705, 18706, 18707 were selected from the ORFs having the nucleotide sequence represented by SEQ ID NO: 1158.

The probes having the nucleotide sequence represented by SEQ ID NOS: 18708, 18709, 18710, 18711, 18712, 18713, 18714, 18715, 18716, 18717, 18718 were selected from the ORFs having the nucleotide sequence represented by SEQ ID NO: 1159.

The probes having the nucleotide sequence represented by SEQ ID NOS: 18719, 18720, 18721, 18722, 18723, 18724, 18725, 18726, 18727, 18728, 18729 were selected from the ORFs having the nucleotide sequence represented by SEQ ID NO: 1160.

The probes having the nucleotide sequence represented by SEQ ID NOS: 18730, 18731, 18732, 18733, 18734, 18735, 18736, 18737, 18738, 18739, 18740 were selected from the ORFs having the nucleotide sequence represented by SEQ ID NO: 1161.

The probes having the nucleotide sequence represented by SEQ ID NOS: 18741, 18742, 18743, 18744, 18745, 18746, 18747, 18748, 18749, 18750, 18751 were selected from the ORFs having the nucleotide sequence represented by SEQ ID NO: 1162.

The probes having the nucleotide sequence represented by SEQ ID NOS: 18752, 18753, 18754, 18755, 18756, 18757, 18758, 18759, 18760, 18761, 18762 were selected from the ORFs having the nucleotide sequence represented by SEQ ID NO: 1163.

The probes having the nucleotide sequence represented by SEQ ID NOS: 18763, 18764, 18765, 18766, 18767, 18768, 18769, 18770, 18771, 18772, 18773 were selected from the ORFs having the nucleotide sequence represented by SEQ ID NO: 1164.

The probes having the nucleotide sequence represented by SEQ ID NOS: 18774, 18775, 18776, 18777, 18778, 18779, 18780, 18781, 18782, 18783, 18784 were selected from the ORFs having the nucleotide sequence represented by SEQ ID NO: 1165.

The probes having the nucleotide sequence represented by SEQ ID NOS: 18785, 18786, 18787, 18788, 18789, 18790, 18791, 18792, 18793, 18794, 18795 were selected from the ORFs having the nucleotide sequence represented by SEQ ID NO: 1166.

The probes having the nucleotide sequence represented by SEQ ID NOS: 18796, 18797, 18798, 18799, 18800, 18801, 18802, 18803, 18804, 18805, 18806 were selected from the ORFs having the nucleotide sequence represented by SEQ ID NO: 1167.

The probes having the nucleotide sequence represented by SEQ ID NOS: 18807, 18808, 18809, 18810, 18811, 18812, 18813, 18814, 18815, 18816, 18817 were selected from the ORFs having the nucleotide sequence represented by SEQ ID NO: 1168.

The probes having the nucleotide sequence represented by SEQ ID NOS: 18818, 18819, 18820, 18821, 18822, 18823, 18824, 18825, 18826, 18827, 18828 were selected from the ORFs having the nucleotide sequence represented by SEQ ID NO: 1169.

The probes having the nucleotide sequence represented by SEQ ID NOS: 18829, 18830, 18831, 18832, 18833, 18834, 18835, 18836, 18837, 18838, 18839 were selected from the ORFs having the nucleotide sequence represented by SEQ ID NO: 1170.

The probes having the nucleotide sequence represented by SEQ ID NOS: 18840, 18841, 18842, 18843, 18844, 18845, 18846, 18847, 18848, 18849, 18850 were selected from the ORFs having the nucleotide sequence represented by SEQ ID NO: 1171.

The probes having the nucleotide sequence represented by SEQ ID NOS: 18851, 18852, 18853, 18854, 18855, 18856, 18857, 18858, 18859, 18860, 18861 were selected from the ORFs having the nucleotide sequence represented by SEQ ID NO: 1172.

The probes having the nucleotide sequence represented by SEQ ID NOS: 18862, 18863, 18864, 18865, 18866, 18867, 18868, 18869, 18870, 18871, 18872 were selected from the ORFs having the nucleotide sequence represented by SEQ ID NO: 1173.

The probes having the nucleotide sequence represented by SEQ ID NOS: 18873, 18874, 18875, 18876, 18877, 18878, 18879, 18880, 18881, 18882, 18883 were selected from the ORFs having the nucleotide sequence represented by SEQ ID NO: 1174.

The probes having the nucleotide sequence represented by SEQ ID NOS: 18884, 18885, 18886, 18887, 18888, 18889, 18890, 18891, 18892, 18893, 18894 were selected from the ORFs having the nucleotide sequence represented by SEQ ID NO: 1175.

The probes having the nucleotide sequence represented by SEQ ID NOS: 18895, 18896, 18897, 18898, 18899, 18900, 18901, 18902, 18903, 18904, 18905 were selected from the ORFs having the nucleotide sequence represented by SEQ ID NO: 1176.

The probes having the nucleotide sequence represented by SEQ ID NOS: 18906, 18907, 18908, 18909, 18910, 18911, 18912, 18913, 18914, 18915, 18916 were selected from the ORFs having the nucleotide sequence represented by SEQ ID NO: 1177.

The probes having the nucleotide sequence represented by SEQ ID NOS: 18917, 18918, 18919, 18920, 18921, 18922, 18923, 18924, 18925, 18926, 18927 were selected from the ORFs having the nucleotide sequence represented by SEQ ID NO: 1178.

The probes having the nucleotide sequence represented by SEQ ID NOS: 18928, 18929, 18930, 18931, 18932, 18933, 18934, 18935, 18936, 18937, 18938 were selected from the ORFs having the nucleotide sequence represented by SEQ ID NO: 1179.

The probes having the nucleotide sequence represented by SEQ ID NOS: 18939, 18940, 18941, 18942, 18943, 18944, 18945, 18946, 18947, 18948, 18949 were selected from the ORFs having the nucleotide sequence represented by SEQ ID NO: 1180.

The probes having the nucleotide sequence represented by SEQ ID NOS: 18950, 18951, 18952, 18953, 18954, 18955, 18956, 18957, 18958, 18959, 18960 were selected from the ORFs having the nucleotide sequence represented by SEQ ID NO: 1181.

The probes having the nucleotide sequence represented by SEQ ID NOS: 18961, 18962, 18963, 18964, 18965, 18966, 18967, 18968, 18969, 18970, 18971 were selected from the ORFs having the nucleotide sequence represented by SEQ ID NO: 1182.

The probes having the nucleotide sequence represented by SEQ ID NOS: 18972, 18973, 18974, 18975, 18976, 18977, 18978, 18979, 18980, 18981, 18982 were selected from the ORFs having the nucleotide sequence represented by SEQ ID NO: 1183.

The probes having the nucleotide sequence represented by SEQ ID NOS: 18983, 18984, 18985, 18986, 18987, 18988, 18989, 18990, 18991, 18992, 18993 were selected from the ORFs having the nucleotide sequence represented by SEQ ID NO: 1184.

The probes having the nucleotide sequence represented by SEQ ID NOS: 18994, 18995, 18996, 18997, 18998, 18999, 19000, 19001, 19002, 19003, 19004 were selected from the ORFs having the nucleotide sequence represented by SEQ ID NO: 1185.

The probes having the nucleotide sequence represented by SEQ ID NOS: 19005, 19006, 19007, 19008, 19009, 19010, 19011, 19012, 19013, 19014, 19015 were selected from the ORFs having the nucleotide sequence represented by SEQ ID NO: 1186.

The probes having the nucleotide sequence represented by SEQ ID NOS: 19016, 19017, 19018, 19019, 19020, 19021, 19022, 19023, 19024, 19025, 19026 were selected from the ORFs having the nucleotide sequence represented by SEQ ID NO: 1187.

The probes having the nucleotide sequence represented by SEQ ID NOS: 19027, 19028, 19029, 19030, 19031, 19032, 19033, 19034, 19035, 19036, 19037 were selected from the ORFs having the nucleotide sequence represented by SEQ ID NO: 1188.

The probes having the nucleotide sequence represented by SEQ ID NOS: 19038, 19039, 19040, 19041, 19042, 19043, 19044, 19045, 19046, 19047, 19048 were selected from the ORFs having the nucleotide sequence represented by SEQ ID NO: 1189.

The probes having the nucleotide sequence represented by SEQ ID NOS: 19049, 19050, 19051, 19052, 19053, 19054, 19055, 19056, 19057, 19058, 19059 were selected from the ORFs having the nucleotide sequence represented by SEQ ID NO: 1190.

The probes having the nucleotide sequence represented by SEQ ID NOS: 19060, 19061, 19062, 19063, 19064, 19065, 19066, 19067, 19068, 19069, 19070 were selected from the ORFs having the nucleotide sequence represented by SEQ ID NO: 1191.

The probes having the nucleotide sequence represented by SEQ ID NOS: 19071, 19072, 19073, 19074, 19075, 19076, 19077, 19078, 19079, 19080, 19081 were selected from the ORFs having the nucleotide sequence represented by SEQ ID NO: 1192.

The probes having the nucleotide sequence represented by SEQ ID NOS: 19082, 19083, 19084, 19085, 19086, 19087, 19088, 19089, 19090, 19091, 19092 were selected from the ORFs having the nucleotide sequence represented by SEQ ID NO: 1193.

The probes having the nucleotide sequence represented by SEQ ID NOS: 19093, 19094, 19095, 19096, 19097, 19098, 19099, 19100, 19101, 19102, 19103 were selected from the ORFs having the nucleotide sequence represented by SEQ ID NO: 1194.

The probes having the nucleotide sequence represented by SEQ ID NOS: 19104, 19105, 19106, 19107, 19108, 19109, 19110, 19111, 19112, 19113, 19114 were selected from the ORFs having the nucleotide sequence represented by SEQ ID NO: 1195.

The probes having the nucleotide sequence represented by SEQ ID NOS: 19115, 19116, 19117, 19118, 19119, 19120, 19121, 19122, 19123, 19124, 19125 were selected from the ORFs having the nucleotide sequence represented by SEQ ID NO: 1196.

The probes having the nucleotide sequence represented by SEQ ID NOS: 19126, 19127, 19128, 19129, 19130, 19131, 19132, 19133, 19134, 19135, 19136 were selected from the ORFs having the nucleotide sequence represented by SEQ ID NO: 1197.

The probes having the nucleotide sequence represented by SEQ. ID NOS: 19137, 19138, 19139, 19140, 19141, 19142, 19143, 19144, 19145, 19146, 19147 were selected from the ORFs having the nucleotide sequence represented by SEQ ID NO: 1198.

The probes having the nucleotide sequence represented by SEQ ID NOS: 19148, 19149, 19150, 19151, 19152, 19153, 19154, 19155, 19156, 19157, 19158 were selected from the ORFs having the nucleotide sequence represented by SEQ ID NO: 1199.

The probes having the nucleotide sequence represented by SEQ ID NOS: 19159, 19160, 19161, 19162, 19163, 19164, 19165, 19166, 19167, 19168, 19169 were selected from the ORFs having the nucleotide sequence represented by SEQ ID NO: 1200.

The probes having the nucleotide sequence represented by SEQ ID NOS: 19170, 19171, 19172, 19173, 19174, 19175, 19176, 19177, 19178, 19179, 19180 were selected from the ORFs having the nucleotide sequence represented by SEQ ID NO: 1201.

The probes having the nucleotide sequence represented by SEQ ID NOS: 19181, 19182, 19183, 19184, 19185, 19186, 19187, 19188, 19189, 19190, 19191 were selected from the ORFs having the nucleotide sequence represented by SEQ ID NO: 1202.

The probes having the nucleotide sequence represented by SEQ ID NOS: 19192, 19193, 19194, 19195, 19196, 19197, 19198, 19199, 19200, 19201, 19202 were selected from the ORFs having the nucleotide sequence represented by SEQ ID NO: 1203.

The probes having the nucleotide sequence represented by SEQ ID NOS: 19203, 19204, 19205, 19206, 19207, 19208, 19209, 19210, 19211, 19212, 19213 were selected from the ORFs having the nucleotide sequence represented by SEQ ID NO: 1204.

The probes having the nucleotide sequence represented by SEQ ID NOS: 19214, 19215, 19216, 19217, 19218, 19219, 19220, 19221, 19222, 19223, 19224 were selected from the ORFs having the nucleotide sequence represented by SEQ ID NO: 1205.

The probes having the nucleotide sequence represented by SEQ ID NOS: 19225, 19226, 19227, 19228, 19229, 19230, 19231, 19232, 19233, 19234, 19235 were selected from the ORFs having the nucleotide sequence represented by SEQ ID NO: 1206.

The probes having the nucleotide sequence represented by SEQ ID NOS: 19236, 19237, 19238, 19239, 19240, 19241, 19242, 19243, 19244, 19245, 19246 were selected from the ORFs having the nucleotide sequence represented by SEQ ID NO: 1207.

The probes having the nucleotide sequence represented by SEQ ID NOS: 19247, 19248, 19249, 19250, 19251, 19252, 19253, 19254, 19255, 19256, 19257 were selected from the ORFs having the nucleotide sequence represented by SEQ ID NO: 1208.

The probes having the nucleotide sequence represented by SEQ ID NOS: 19258, 19259, 19260, 19261, 19262, 19263, 19264, 19265, 19266, 19267, 19268 were selected from the ORFs having the nucleotide sequence represented by SEQ ID NO: 1209.

The probes having the nucleotide sequence represented by SEQ ID NOS: 19269, 19270, 19271, 19272, 19273, 19274, 19275, 19276, 19277, 19278, 19279 were selected from the ORFs having the nucleotide sequence represented by SEQ ID NO: 1210.

The probes having the nucleotide sequence represented by SEQ ID NOS: 19280, 19281, 19282, 19283, 19284, 19285, 19286, 19287, 19288, 19289, 19290 were selected from the ORFs having the nucleotide sequence represented by SEQ ID NO: 1211.

The probes having the nucleotide sequence represented by SEQ ID NOS: 19291, 19292, 19293, 19294, 19295, 19296, 19297, 19298, 19299, 19300, 19301 were selected from the ORFs having the nucleotide sequence represented by SEQ ID NO: 1212.

The probes having the nucleotide sequence represented by SEQ ID NOS: 19302, 19303, 19304, 19305, 19306, 19307, 19308, 19309, 19310, 19311, 19312 were selected from the ORFs having the nucleotide sequence represented by SEQ ID NO: 1213.

The probes having the nucleotide sequence represented by SEQ ID NOS: 19313, 19314, 19315, 19316, 19317, 19318, 19319, 19320, 19321, 19322, 19323 were selected from the ORFs having the nucleotide sequence represented by SEQ ID NO: 1214.

The probes having the nucleotide sequence represented by SEQ ID NOS: 19324, 19325, 19326, 19327, 19328, 19329, 19330, 19331, 19332, 19333, 19334 were selected from the ORFs having the nucleotide sequence represented by SEQ ID NO: 1215.

The probes having the nucleotide sequence represented by SEQ ID NOS: 19335, 19336, 19337, 19338, 19339, 19340, 19341, 19342, 19343, 19344, 19345 were selected from the ORFs having the nucleotide sequence represented by SEQ ID NO: 1216.

The probes having the nucleotide sequence represented by SEQ ID NOS: 19346, 19347, 19348, 19349, 19350, 19351, 19352, 19353, 19354, 19355, 19356 were selected from the ORFs having the nucleotide sequence represented by SEQ ID NO: 1217.

The probes having the nucleotide sequence represented by SEQ ID NOS: 19357, 19358, 19359, 19360, 19361, 19362, 19363, 19364, 19365, 19366, 19367 were selected from the ORFs having the nucleotide sequence represented by SEQ ID NO: 1218.

The probes having the nucleotide sequence represented by SEQ ID NOS: 19368, 19369, 19370, 19371, 19372, 19373, 19374, 19375, 19376, 19377, 19378 were selected from the ORFs having the nucleotide sequence represented by SEQ ID NO: 1219.

The probes having the nucleotide sequence represented by SEQ ID NOS: 19379, 19380, 19381, 19382, 19383, 19384, 19385, 19386, 19387, 19388, 19389 were selected from the ORFs having the nucleotide sequence represented by SEQ ID NO: 1220.

The probes having the nucleotide sequence represented by SEQ ID NOS: 19390, 19391, 19392, 19393, 19394, 19395, 19396, 19397, 19398, 19399, 19400 were selected from the ORFs having the nucleotide sequence represented by SEQ ID NO: 1221.

The probes having the nucleotide sequence represented by SEQ ID NOS: 19401, 19402, 19403, 19404, 19405, 19406, 19407, 19408, 19409, 19410, 19411 were selected from the ORFs having the nucleotide sequence represented by SEQ ID NO: 1222.

The probes having the nucleotide sequence represented by SEQ ID NOS: 19412, 19413, 19414, 19415, 19416, 19417, 19418, 19419, 19420, 19421, 19422 were selected from the ORFs having the nucleotide sequence represented by SEQ ID NO: 1223.

The probes having the nucleotide sequence represented by SEQ ID NOS: 19423, 19424, 19425, 19426, 19427, 19428, 19429, 19430, 19431, 19432, 19433 were selected from the ORFs having the nucleotide sequence represented by SEQ ID NO: 1224.

The probes having the nucleotide sequence represented by SEQ ID NOS: 19434, 19435, 19436, 19437, 19438, 19439, 19440, 19441, 19442, 19443, 19444 were selected from the ORFs having the nucleotide sequence represented by SEQ ID NO: 1225.

The probes having the nucleotide sequence represented by SEQ ID NOS: 19445, 19446, 19447, 19448, 19449, 19450, 19451, 19452, 19453, 19454, 19455 were selected from the ORFs having the nucleotide sequence represented by SEQ ID NO: 1226.

The probes having the nucleotide sequence represented by SEQ ID NOS: 19456, 19457, 19458, 19459, 19460, 19461, 19462, 19463, 19464, 19465, 19466 were selected from the ORFs having the nucleotide sequence represented by SEQ ID NO: 1227.

The probes having the nucleotide sequence represented by SEQ ID NOS: 19467, 19468, 19469, 19470, 19471, 19472, 19473, 19474, 19475, 19476, 19477 were selected from the ORFs having the nucleotide sequence represented by SEQ ID NO: 1228.

The probes having the nucleotide sequence represented by SEQ ID NOS: 19478, 19479, 19480, 19481, 19482, 19483, 19484, 19485, 19486, 19487, 19488 were selected from the ORFs having the nucleotide sequence represented by SEQ ID NO: 1229.

The probes having the nucleotide sequence represented by SEQ ID NOS: 19489, 19490, 19491, 19492, 19493, 19494, 19495, 19496, 19497, 19498, 19499 were selected from the ORFs having the nucleotide sequence represented by SEQ ID NO: 1230.

The probes having the nucleotide sequence represented by SEQ ID NOS: 19500, 19501, 19502, 19503, 19504, 19505, 19506, 19507, 19508, 19509, 19510 were selected from the ORFs having the nucleotide sequence represented by SEQ ID NO: 1231.

The probes having the nucleotide sequence represented by SEQ ID NOS: 19511, 19512, 19513, 19514, 19515, 19516, 19517, 19518, 19519, 19520, 19521 were selected from the ORFs having the nucleotide sequence represented by SEQ ID NO: 1232.

The probes having the nucleotide sequence represented by SEQ ID NOS: 19522, 19523, 19524, 19525, 19526, 19527, 19528, 19529, 19530, 19531, 19532 were selected from the ORFs having the nucleotide sequence represented by SEQ ID NO: 1233.

The probes having the nucleotide sequence represented by SEQ ID NOS: 19533, 19534, 19535, 19536, 19537, 19538, 19539, 19540, 19541, 19542, 19543 were selected from the ORFs having the nucleotide sequence represented by SEQ ID NO: 1234.

The probes having the nucleotide sequence represented by SEQ ID NOS: 19544, 19545, 19546, 19547, 19548, 19549, 19550, 19551, 19552, 19553, 19554 were selected from the ORFs having the nucleotide sequence represented by SEQ ID NO: 1235.

The probes having the nucleotide sequence represented by SEQ ID NOS: 19555, 19556, 19557, 19558, 19559, 19560, 19561, 19562, 19563, 19564, 19565 were selected from the ORFs having the nucleotide sequence represented by SEQ ID NO: 1236.

The probes having the nucleotide sequence represented by SEQ ID NOS: 19566, 19567, 19568, 19569, 19570, 19571, 19572, 19573, 19574, 19575, 19576 were selected from the ORFs having the nucleotide sequence represented by SEQ ID NO: 1237.

The probes having the nucleotide sequence represented by SEQ ID NOS: 19577, 19578, 19579, 19580, 19581, 19582, 19583, 19584, 19585, 19586, 19587 were selected from the ORFs having the nucleotide sequence represented by SEQ ID NO: 1238.

The probes having the nucleotide sequence represented by SEQ ID NOS: 19588, 19589, 19590, 19591, 19592, 19593, 19594, 19595, 19596, 19597, 19598 were selected from the ORFs having the nucleotide sequence represented by SEQ ID NO: 1239.

The probes having the nucleotide sequence represented by SEQ ID NOS: 19599, 19600, 19601, 19602, 19603, 19604, 19605, 19606, 19607, 19608, 19609 were selected from the ORFs having the nucleotide sequence represented by SEQ ID NO: 1240.

The probes having the nucleotide sequence represented by SEQ ID NOS: 19610, 19611, 19612, 19613, 19614, 19615, 19616, 19617, 19618, 19619, 19620 were selected from the ORFs having the nucleotide sequence represented by SEQ ID NO: 1241.

The probes having the nucleotide sequence represented by SEQ ID NOS: 19621, 19622, 19623, 19624, 19625, 19626, 19627, 19628, 19629, 19630, 19631 were selected from the ORFs having the nucleotide sequence represented by SEQ ID NO: 1242.

The probes having the nucleotide sequence represented by SEQ ID NOS: 19632, 19633, 19634, 19635, 19636, 19637, 19638, 19639, 19640, 19641, 19642 were selected from the ORFs having the nucleotide sequence represented by SEQ ID NO: 1243.

The probes having the nucleotide sequence represented by SEQ ID NOS: 19643, 19644, 19645, 19646, 19647, 19648, 19649, 19650, 19651, 19652, 19653 were selected from the ORFs having the nucleotide sequence represented by SEQ ID NO: 1244.

The probes having the nucleotide sequence represented by SEQ ID NOS: 19654, 19655, 19656, 19657, 19658, 19659, 19660, 19661, 19662, 19663, 19664 were selected from the ORFs having the nucleotide sequence represented by SEQ ID NO: 1245.

The probes having the nucleotide sequence represented by SEQ ID NOS: 19665, 19666, 19667, 19668, 19669, 19670, 19671, 19672, 19673, 19674, 19675 were selected from the ORFs having the nucleotide sequence represented by SEQ ID NO: 1246.

The probes having the nucleotide sequence represented by SEQ ID NOS: 19676, 19677, 19678, 19679, 19680, 19681, 19682, 19683, 19684, 19685, 19686 were selected from the ORFs having the nucleotide sequence represented by SEQ ID NO: 1247.

The probes having the nucleotide sequence represented by SEQ ID NOS: 19687, 19688, 19689, 19690, 19691, 19692, 19693, 19694, 19695, 19696, 19697 were selected from the ORFs having the nucleotide sequence represented by SEQ ID NO: 1248.

The probes having the nucleotide sequence represented by SEQ. ID NOS: 19698, 19699, 19700, 19701, 19702, 19703, 19704, 19705, 19706, 19707, 19708 were selected from the ORFs having the nucleotide sequence represented by SEQ ID NO: 1249.

The probes having the nucleotide sequence represented by SEQ ID NOS: 19709, 19710, 19711, 19712, 19713, 19714, 19715, 19716, 19717, 19718, 19719 were selected from the ORFs having the nucleotide sequence represented by SEQ ID NO: 1250.

The probes having the nucleotide sequence represented by SEQ ID NOS: 19720, 19721, 19722, 19723, 19724, 19725, 19726, 19727, 19728, 19729, 19730 were selected from the ORFs having the nucleotide sequence represented by SEQ ID NO: 1251.

The probes having the nucleotide sequence represented by SEQ ID NOS: 19731, 19732, 19733, 19734, 19735, 19736, 19737, 19738, 19739, 19740, 19741 were selected from the ORFs having the nucleotide sequence represented by SEQ ID NO: 1252.

The probes having the nucleotide sequence represented by SEQ ID NOS: 19742, 19743, 19744, 19745, 19746, 19747, 19748, 19749, 19750, 19751, 19752 were selected from the ORFs having the nucleotide sequence represented by SEQ ID NO: 1253.

The probes having the nucleotide sequence represented by SEQ ID NOS: 19753, 19754, 19755, 19756, 19757, 19758, 19759, 19760, 19761, 19762, 19763 were selected from the ORFs having the nucleotide sequence represented by SEQ ID NO: 1254.

The probes having the nucleotide sequence represented by SEQ ID NOS: 19764, 19765, 19766, 19767, 19768, 19769, 19770, 19771, 19772, 19773, 19774 were selected from the ORFs having the nucleotide sequence represented by SEQ ID NO: 1255.

The probes having the nucleotide sequence represented by SEQ ID NOS: 19775, 19776, 19777, 19778, 19779, 19780, 19781, 19782, 19783, 19784, 19785 were selected from the ORFs having the nucleotide sequence represented by SEQ ID NO: 1256.

The probes having the nucleotide sequence represented by SEQ ID NOS: 19786, 19787, 19788, 19789, 19790, 19791, 19792, 19793, 19794, 19795, 19796 were selected from the ORFs having the nucleotide sequence represented by SEQ ID NO: 1257.

The probes having the nucleotide sequence represented by SEQ ID NOS: 19797, 19798, 19799, 19800, 19801, 19802, 19803, 19804, 19805, 19806, 19807 were selected from the ORFs having the nucleotide sequence represented by SEQ ID NO: 1258.

The probes having the nucleotide sequence represented by SEQ ID NOS: 19808, 19809, 19810, 19811, 19812, 19813, 19814, 19815, 19816, 19817, 19818 were selected from the ORFs having the nucleotide sequence represented by SEQ ID NO: 1259.

The probes having the nucleotide sequence represented by SEQ ID NOS: 19819, 19820, 19821, 19822, 19823, 19824, 19825, 19826, 19827, 19828, 19829 were selected from the ORFs having the nucleotide sequence represented by SEQ ID NO: 1260.

The probes having the nucleotide sequence represented by SEQ ID NOS: 19830, 19831, 19832, 19833, 19834, 19835, 19836, 19837, 19838, 19839, 19840 were selected from the ORFs having the nucleotide sequence represented by SEQ ID NO: 1261.

The probes having the nucleotide sequence represented by SEQ ID NOS: 19841, 19842, 19843, 19844, 19845, 19846, 19847, 19848, 19849, 19850, 19851 were selected from the ORFs having the nucleotide sequence represented by SEQ ID NO: 1262.

The probes having the nucleotide sequence represented by SEQ ID NOS: 19852, 19853, 19854, 19855, 19856, 19857, 19858, 19859, 19860, 19861, 19862 were selected from the ORFs having the nucleotide sequence represented by SEQ ID NO: 1263.

The probes having the nucleotide sequence represented by SEQ ID NOS: 19863, 19864, 19865, 19866, 19867, 19868, 19869, 19870, 19871, 19872, 19873 were selected from the ORFs having the nucleotide sequence represented by SEQ ID NO: 1264.

The probes having the nucleotide sequence represented by SEQ ID NOS: 19874, 19875, 19876, 19877, 19878, 19879, 19880, 19881, 19882, 19883, 19884 were selected from the ORFs having the nucleotide sequence represented by SEQ ID NO: 1265.

The probes having the nucleotide sequence represented by SEQ ID NOS: 19885, 19886, 19887, 19888, 19889, 19890, 19891, 19892, 19893, 19894, 19895 were selected from the ORFs having the nucleotide sequence represented by SEQ ID NO: 1266.

The probes having the nucleotide sequence represented by SEQ ID NOS: 19896, 19897, 19898, 19899, 19900, 19901, 19902, 19903, 19904, 19905, 19906 were selected from the ORFs having the nucleotide sequence represented by SEQ ID NO: 1267.

The probes having the nucleotide sequence represented by SEQ ID NOS: 19907, 19908, 19909, 19910, 19911, 19912, 19913, 19914, 19915, 19916, 19917 were selected from the ORFs having the nucleotide sequence represented by SEQ ID NO: 1268.

The probes having the nucleotide sequence represented by SEQ ID NOS: 19918, 19919, 19920, 19921, 19922, 19923, 19924, 19925, 19926, 19927, 19928 were selected from the ORFs having the nucleotide sequence represented by SEQ ID NO: 1269.

The probes having the nucleotide sequence represented by SEQ ID NOS: 19929, 19930, 19931, 19932, 19933, 19934, 19935, 19936, 19937, 19938, 19939 were selected from the ORFs having the nucleotide sequence represented by SEQ ID NO: 1270.

The probes having the nucleotide sequence represented by SEQ ID NOS: 19940, 19941, 19942, 19943, 19944, 19945, 19946, 19947, 19948, 19949, 19950 were selected from the ORFs having the nucleotide sequence represented by SEQ ID NO: 1271.

The probes having the nucleotide sequence represented by SEQ ID NOS: 19951, 19952, 19953, 19954, 19955, 19956, 19957, 19958, 19959, 19960, 19961 were selected from the ORFs having the nucleotide sequence represented by SEQ ID NO: 1272.

The probes having the nucleotide sequence represented by SEQ ID NOS: 19962, 19963, 19964, 19965, 19966, 19967, 19968, 19969, 19970, 19971, 19972 were selected from the ORFs having the nucleotide sequence represented by SEQ ID NO: 1273.

The probes having the nucleotide sequence represented by SEQ ID NOS: 19973, 19974, 19975, 19976, 19977, 19978, 19979, 19980, 19981, 19982, 19983 were selected from the ORFs having the nucleotide sequence represented by SEQ ID NO: 1274.

The probes having the nucleotide sequence represented by SEQ ID NOS: 19984, 19985, 19986, 19987, 19988, 19989, 19990, 19991, 19992, 19993, 19994 were selected from the ORFs having the nucleotide sequence represented by SEQ ID NO: 1275.

The probes having the nucleotide sequence represented by SEQ ID NOS: 19995, 19996, 19997, 19998, 19999, 20000, 20001, 20002, 20003, 20004, 20005 were selected from the ORFs having the nucleotide sequence represented by SEQ ID NO: 1276.

The probes having the nucleotide sequence represented by SEQ ID NOS: 20006, 20007, 20008, 20009, 20010, 20011, 20012, 20013, 20014, 20015, 20016 were selected from the ORFs having the nucleotide sequence represented by SEQ ID NO: 1277.

The probes having the nucleotide sequence represented by SEQ ID NOS: 20017, 20018, 20019, 20020, 20021, 20022, 20023, 20024, 20025, 20026, 20027 were selected from the ORFs having the nucleotide sequence represented by SEQ ID NO: 1278.

The probes having the nucleotide sequence represented by SEQ ID NOS: 20028, 20029, 20030, 20031, 20032, 20033, 20034, 20035, 20036, 20037, 20038 were selected from the ORFs having the nucleotide sequence represented by SEQ ID NO: 1279.

The probes having the nucleotide sequence represented by SEQ ID NOS: 20039, 20040, 20041, 20042, 20043, 20044, 20045, 20046, 20047, 20048, 20049 were selected from the ORFs having the nucleotide sequence represented by SEQ ID NO: 1280.

The probes having the nucleotide sequence represented by SEQ ID NOS: 20050, 20051, 20052, 20053, 20054, 20055, 20056, 20057, 20058, 20059, 20060 were selected from the ORFs having the nucleotide sequence represented by SEQ ID NO: 1281.

The probes having the nucleotide sequence represented by SEQ ID NOS: 20061, 20062, 20063, 20064, 20065, 20066, 20067, 20068, 20069, 20070, 20071 were selected from the ORFs having the nucleotide sequence represented by SEQ ID NO: 1282.

The probes having the nucleotide sequence represented by SEQ ID NOS: 20072, 20073, 20074, 20075, 20076, 20077, 20078, 20079, 20080, 20081, 20082 were selected from the ORFs having the nucleotide sequence represented by SEQ ID NO: 1283.

The probes having the nucleotide sequence represented by SEQ ID NOS: 20083, 20084, 20085, 20086, 20087, 20088, 20089, 20090, 20091, 20092, 20093 were selected from the ORFs having the nucleotide sequence represented by SEQ ID NO: 1284.

The probes having the nucleotide sequence represented by SEQ ID NOS: 20094, 20095, 20096, 20097, 20098, 20099, 20100, 20101, 20102, 20103, 20104 were selected from the ORFs having the nucleotide sequence represented by SEQ ID NO: 1285.

The probes having the nucleotide sequence represented by SEQ ID NOS: 20105, 20106, 20107, 20108, 20109, 20110, 20111, 20112, 20113, 20114, 20115 were selected from the ORFs having the nucleotide sequence represented by SEQ ID NO: 1286.

The probes having the nucleotide sequence represented by SEQ ID NOS: 20116, 20117, 20118, 20119, 20120, 20121, 20122, 20123, 20124, 20125, 20126 were selected from the ORFs having the nucleotide sequence represented by SEQ ID NO: 1287.

The probes having the nucleotide sequence represented by SEQ ID NOS: 20127, 20128, 20129, 20130, 20131, 20132, 20133, 20134, 20135, 20136, 20137 were selected from the ORFs having the nucleotide sequence represented by SEQ ID NO: 1288.

The probes having the nucleotide sequence represented by SEQ ID NOS: 20138, 20139, 20140, 20141, 20142, 20143, 20144, 20145, 20146, 20147, 20148 were selected from the ORFs having the nucleotide sequence represented by SEQ ID NO: 1289.

The probes having the nucleotide sequence represented by SEQ ID NOS: 20149, 20150, 20151, 20152, 20153, 20154, 20155, 20156, 20157, 20158, 20159 were selected from the ORFs having the nucleotide sequence represented by SEQ ID NO: 1290.

The probes having the nucleotide sequence represented by SEQ ID NOS: 20160, 20161, 20162, 20163, 20164, 20165, 20166, 20167, 20168, 20169, 20170 were selected from the ORFs having the nucleotide sequence represented by SEQ ID NO: 1291.

The probes having the nucleotide sequence represented by SEQ ID NOS: 20171, 20172, 20173, 20174, 20175, 20176, 20177, 20178, 20179, 20180, 20181 were selected from the ORFs having the nucleotide sequence represented by SEQ ID NO: 1292.

The probes having the nucleotide sequence represented by SEQ ID NOS: 20182, 20183, 20184, 20185, 20186, 20187, 20188, 20189, 20190, 20191, 20192 were selected from the ORFs having the nucleotide sequence represented by SEQ ID NO: 1293.

The probes having the nucleotide sequence represented by SEQ ID NOS: 20193, 20194, 20195, 20196, 20197, 20198, 20199, 20200, 20201, 20202, 20203 were selected from the ORFs having the nucleotide sequence represented by SEQ ID NO: 1294.

The probes having the nucleotide sequence represented by SEQ ID NOS: 20204, 20205, 20206, 20207, 20208, 20209, 20210, 20211, 20212, 20213, 20214 were selected from the ORFs having the nucleotide sequence represented by SEQ ID NO: 1295.

The probes having the nucleotide sequence represented by SEQ ID NOS: 20215, 20216, 20217, 20218, 20219, 20220, 20221, 20222, 20223, 20224, 20225 were selected from the ORFs having the nucleotide sequence represented by SEQ ID NO: 1296.

The probes having the nucleotide sequence represented by SEQ ID NOS: 20226, 20227, 20228, 20229, 20230, 20231, 20232, 20233, 20234, 20235, 20236 were selected from the ORFs having the nucleotide sequence represented by SEQ ID NO: 1297.

The probes having the nucleotide sequence represented by SEQ ID NOS: 20237, 20238, 20239, 20240, 20241, 20242, 20243, 20244, 20245, 20246, 20247 were selected from the ORFs having the nucleotide sequence represented by SEQ ID NO: 1298.

The probes having the nucleotide sequence represented by SEQ ID NOS: 20248, 20249, 20250, 20251, 20252, 20253, 20254, 20255, 20256, 20257, 20258 were selected from the ORFs having the nucleotide sequence represented by SEQ ID NO: 1299.

The probes having the nucleotide sequence represented by SEQ ID NOS: 20259, 20260, 20261, 20262, 20263, 20264, 20265, 20266, 20267, 20268, 20269 were selected from the ORFs having the nucleotide sequence represented by SEQ ID NO: 1300.

The probes having the nucleotide sequence represented by SEQ ID NOS: 20270, 20271, 20272, 20273, 20274, 20275, 20276, 20277, 20278, 20279, 20280 were selected from the ORFs having the nucleotide sequence represented by SEQ ID NO: 1301.

The probes having the nucleotide sequence represented by SEQ ID NOS: 20281, 20282, 20283, 20284, 20285, 20286, 20287, 20288, 20289, 20290, 20291 were selected from the ORFs having the nucleotide sequence represented by SEQ ID NO: 1302.

The probes having the nucleotide sequence represented by SEQ ID NOS: 20292, 20293, 20294, 20295, 20296, 20297, 20298, 20299, 20300, 20301, 20302 were selected from the ORFs having the nucleotide sequence represented by SEQ ID NO: 1303.

The probes having the nucleotide sequence represented by SEQ ID NOS: 20303, 20304, 20305, 20306, 20307, 20308, 20309, 20310, 20311, 20312, 20313 were selected from the ORFs having the nucleotide sequence represented by SEQ ID NO: 1304.

The probes having the nucleotide sequence represented by SEQ ID NOS: 20314, 20315, 20316, 20317, 20318, 20319, 20320, 20321, 20322, 20323, 20324 were selected from the ORFs having the nucleotide sequence represented by SEQ ID NO: 1305.

The probes having the nucleotide sequence represented by SEQ ID NOS: 20325, 20326, 20327, 20328, 20329, 20330, 20331, 20332, 20333, 20334, 20335 were selected from the ORFs having the nucleotide sequence represented by SEQ ID NO: 1306.

The probes having the nucleotide sequence represented by SEQ ID NOS: 20336, 20337, 20338, 20339, 20340, 20341, 20342, 20343, 20344, 20345, 20346 were selected from the ORFs having the nucleotide sequence represented by SEQ ID NO: 1307.

The probes having the nucleotide sequence represented by SEQ ID NOS: 20347, 20348, 20349, 20350, 20351, 20352, 20353, 20354, 20355, 20356, 20357 were selected from the ORFs having the nucleotide sequence represented by SEQ ID NO: 1308.

The probes having the nucleotide sequence represented by SEQ ID NOS: 20358, 20359, 20360, 20361, 20362, 20363, 20364, 20365, 20366, 20367, 20368 were selected from the ORFs having the nucleotide sequence represented by SEQ ID NO: 1309.

The probes having the nucleotide sequence represented by SEQ ID NOS: 20369, 20370, 20371, 20372, 20373, 20374, 20375, 20376, 20377, 20378, 20379 were selected from the ORFs having the nucleotide sequence represented by SEQ ID NO: 1310.

The probes having the nucleotide sequence represented by SEQ ID NOS: 20380, 20381, 20382, 20383, 20384, 20385, 20386, 20387, 20388, 20389, 20390 were selected from the ORFs having the nucleotide sequence represented by SEQ ID NO: 1311.

The probes having the nucleotide sequence represented by SEQ ID NOS: 20391, 20392, 20393, 20394, 20395, 20396, 20397, 20398, 20399, 20400, 20401 were selected from the ORFs having the nucleotide sequence represented by SEQ ID NO: 1312.

The probes having the nucleotide sequence represented by SEQ ID NOS: 20402, 20403, 20404, 20405, 20406, 20407, 20408, 20409, 20410, 20411, 20412 were selected from the ORFs having the nucleotide sequence represented by SEQ ID NO: 1313.

The probes having the nucleotide sequence represented by SEQ ID NOS: 20413, 20414, 20415, 20416, 20417, 20418, 20419, 20420, 20421, 20422, 20423 were selected from the ORFs having the nucleotide sequence represented by SEQ ID NO: 1314.

The probes having the nucleotide sequence represented by SEQ ID NOS: 20424, 20425, 20426, 20427, 20428, 20429, 20430, 20431, 20432, 20433, 20434 were selected from the ORFs having the nucleotide sequence represented by SEQ ID NO: 1315.

The probes having the nucleotide sequence represented by SEQ ID NOS: 20435, 20436, 20437, 20438, 20439, 20440, 20441, 20442, 20443, 20444, 20445 were selected from the ORFs having the nucleotide sequence represented by SEQ ID NO: 1316.

The probes having the nucleotide sequence represented by SEQ ID NOS: 20446, 20447, 20448, 20449, 20450, 20451, 20452, 20453, 20454, 20455, 20456 were selected from the ORFs having the nucleotide sequence represented by SEQ ID NO: 1317.

The probes having the nucleotide sequence represented by SEQ ID NOS: 20457, 20458, 20459, 20460, 20461, 20462, 20463, 20464, 20465, 20466, 20467 were selected from the ORFs having the nucleotide sequence represented by SEQ ID NO: 1318.

The probes having the nucleotide sequence represented by SEQ ID NOS: 20468, 20469, 20470, 20471, 20472, 20473, 20474, 20475, 20476, 20477, 20478 were selected from the ORFs having the nucleotide sequence represented by SEQ ID NO: 1319.

The probes having the nucleotide sequence represented by SEQ ID NOS: 20479, 20480, 20481, 20482, 20483, 20484, 20485, 20486, 20487, 20488, 20489 were selected from the ORFs having the nucleotide sequence represented by SEQ ID NO: 1320.

The probes having the nucleotide sequence represented by SEQ ID NOS: 20490, 20491, 20492, 20493, 20494, 20495, 20496, 20497, 20498, 20499, 20500 were selected from the ORFs having the nucleotide sequence represented by SEQ ID NO: 1321.

The probes having the nucleotide sequence represented by SEQ ID NOS: 20501, 20502, 20503, 20504, 20505, 20506, 20507, 20508, 20509, 20510, 20511 were selected from the ORFs having the nucleotide sequence represented by SEQ ID NO: 1322.

The probes having the nucleotide sequence represented by SEQ ID NOS: 20512, 20513, 20514, 20515, 20516, 20517, 20518, 20519, 20520, 20521, 20522 were selected from the ORFs having the nucleotide sequence represented by SEQ ID NO: 1323.

The probes having the nucleotide sequence represented by SEQ ID NOS: 20523, 20524, 20525, 20526, 20527, 20528, 20529, 20530, 20531, 20532, 20533 were selected from the ORFs having the nucleotide sequence represented by SEQ ID NO: 1324.

The probes having the nucleotide sequence represented by SEQ ID NOS: 20534, 20535, 20536, 20537, 20538, 20539, 20540, 20541, 20542, 20543, 20544 were selected from the ORFs having the nucleotide sequence represented by SEQ ID NO: 1325.

The probes having the nucleotide sequence represented by SEQ ID NOS: 20545, 20546, 20547, 20548, 20549, 20550, 20551, 20552, 20553, 20554, 20555 were selected from the ORFs having the nucleotide sequence represented by SEQ ID NO: 1326.

The probes having the nucleotide sequence represented by SEQ ID NOS: 20556, 20557, 20558, 20559, 20560, 20561, 20562, 20563, 20564, 20565, 20566 were selected from the ORFs having the nucleotide sequence represented by SEQ ID NO: 1327.

The probes having the nucleotide sequence represented by SEQ ID NOS: 20567, 20568, 20569, 20570, 20571, 20572, 20573, 20574, 20575, 20576, 20577 were selected from the ORFs having the nucleotide sequence represented by SEQ ID NO: 1328.

The probes having the nucleotide sequence represented by SEQ ID NOS: 20578, 20579, 20580, 20581, 20582, 20583, 20584, 20585, 20586, 20587, 20588 were selected from the ORFs having the nucleotide sequence represented by SEQ ID NO: 1329.

The probes having the nucleotide sequence represented by SEQ ID NOS: 20589, 20590, 20591, 20592, 20593, 20594, 20595, 20596, 20597, 20598, 20599 were selected from the ORFs having the nucleotide sequence represented by SEQ ID NO: 1330.

The probes having the nucleotide sequence represented by SEQ ID NOS: 20600, 20601, 20602, 20603, 20604, 20605, 20606, 20607, 20608, 20609, 20610 were selected from the ORFs having the nucleotide sequence represented by SEQ ID NO: 1331.

The probes having the nucleotide sequence represented by SEQ ID NOS: 20611, 20612, 20613, 20614, 20615, 20616, 20617, 20618, 20619, 20620, 20621 were selected from the ORFs having the nucleotide sequence represented by SEQ ID NO: 1332.

The probes having the nucleotide sequence represented by SEQ ID NOS: 20622, 20623, 20624, 20625, 20626, 20627, 20628, 20629, 20630, 20631, 20632 were selected from the ORFs having the nucleotide sequence represented by SEQ ID NO: 1333.

The probes having the nucleotide sequence represented by SEQ ID NOS: 20633, 20634, 20635, 20636, 20637, 20638, 20639, 20640, 20641, 20642, 20643 were selected from the ORFs having the nucleotide sequence represented by SEQ ID NO: 1334.

The probes having the nucleotide sequence represented by SEQ ID NOS: 20644, 20645, 20646, 20647, 20648, 20649, 20650, 20651, 20652, 20653, 20654 were selected from the ORFs having the nucleotide sequence represented by SEQ ID NO: 1335.

The probes having the nucleotide sequence represented by SEQ ID NOS: 20655, 20656, 20657, 20658, 20659, 20660, 20661, 20662, 20663, 20664, 20665 were selected from the ORFs having the nucleotide sequence represented by SEQ ID NO: 1336.

The probes having the nucleotide sequence represented by SEQ ID NOS: 20666, 20667, 20668, 20669, 20670, 20671, 20672, 20673, 20674, 20675, 20676 were selected from the ORFs having the nucleotide sequence represented by SEQ ID NO: 1337.

The probes having the nucleotide sequence represented by SEQ ID NOS: 20677, 20678, 20679, 20680, 20681, 20682, 20683, 20684, 20685, 20686, 20687 were selected from the ORFs having the nucleotide sequence represented by SEQ ID NO: 1338.

The probes having the nucleotide sequence represented by SEQ ID NOS: 20688, 20689, 20690, 20691, 20692, 20693, 20694, 20695, 20696, 20697, 20698 were selected from the ORFs having the nucleotide sequence represented by SEQ ID NO: 1339.

The probes having the nucleotide sequence represented by SEQ ID NOS: 20699, 20700, 20701, 20702, 20703, 20704, 20705, 20706, 20707, 20708, 20709 were selected from the ORFs having the nucleotide sequence represented by SEQ ID NO: 1340.

The probes having the nucleotide sequence represented by SEQ ID NOS: 20710, 20711, 20712, 20713, 20714, 20715, 20716, 20717, 20718, 20719, 20720 were selected from the ORFs having the nucleotide sequence represented by SEQ ID NO: 1341.

The probes having the nucleotide sequence represented by SEQ ID NOS: 20721, 20722, 20723, 20724, 20725, 20726, 20727, 20728, 20729, 20730, 20731 were selected from the ORFs having the nucleotide sequence represented by SEQ ID NO: 1342.

The probes having the nucleotide sequence represented by SEQ ID NOS: 20732, 20733, 20734, 20735, 20736, 20737, 20738, 20739, 20740, 20741, 20742 were selected from the ORFs having the nucleotide sequence represented by SEQ ID NO: 1343.

The probes having the nucleotide sequence represented by SEQ ID NOS: 20743, 20744, 20745, 20746, 20747, 20748, 20749, 20750, 20751, 20752, 20753 were selected from the ORFs having the nucleotide sequence represented by SEQ ID NO: 1344.

The probes having the nucleotide sequence represented by SEQ ID NOS: 20754, 20755, 20756, 20757, 20758, 20759, 20760, 20761, 20762, 20763, 20764 were selected from the ORFs having the nucleotide sequence represented by SEQ ID NO: 1345.

The probes having the nucleotide sequence represented by SEQ ID NOS: 20765, 20766, 20767, 20768, 20769, 20770, 20771, 20772, 20773, 20774, 20775 were selected from the ORFs having the nucleotide sequence represented by SEQ ID NO: 1346.

The probes having the nucleotide sequence represented by SEQ ID NOS: 20776, 20777, 20778, 20779, 20780, 20781, 20782, 20783, 20784, 20785, 20786 were selected from the ORFs having the nucleotide sequence represented by SEQ ID NO: 1347.

The probes having the nucleotide sequence represented by SEQ ID NOS: 20787, 20788, 20789, 20790, 20791, 20792, 20793, 20794, 20795, 20796, 20797 were selected from the ORFs having the nucleotide sequence represented by SEQ ID NO: 1348.

The probes having the nucleotide sequence represented by SEQ ID NOS: 20798, 20799, 20800, 20801, 20802, 20803, 20804, 20805, 20806, 20807, 20808 were selected from the ORFs having the nucleotide sequence represented by SEQ ID NO: 1349.

The probes having the nucleotide sequence represented by SEQ ID NOS: 20809, 20810, 20811, 20812, 20813, 20814, 20815, 20816, 20817, 20818, 20819 were selected from the ORFs having the nucleotide sequence represented by SEQ ID NO: 1350.

The probes having the nucleotide sequence represented by SEQ ID NOS: 20820, 20821, 20822, 20823, 20824, 20825, 20826, 20827, 20828, 20829, 20830 were selected from the ORFs having the nucleotide sequence represented by SEQ ID NO: 1351.

The probes having the nucleotide sequence represented by SEQ ID NOS: 20831, 20832, 20833, 20834, 20835, 20836, 20837, 20838, 20839, 20840, 20841 were selected from the ORFs having the nucleotide sequence represented by SEQ ID NO: 1352.

The probes having the nucleotide sequence represented by SEQ ID NOS: 20842, 20843, 20844, 20845, 20846, 20847, 20848, 20849, 20850, 20851, 20852 were selected from the ORFs having the nucleotide sequence represented by SEQ ID NO: 1353.

The probes having the nucleotide sequence represented by SEQ ID NOS: 20853, 20854, 20855, 20856, 20857, 20858, 20859, 20860, 20861, 20862, 20863 were selected from the ORFs having the nucleotide sequence represented by SEQ ID NO: 1354.

The probes having the nucleotide sequence represented by SEQ ID NOS: 20864, 20865, 20866, 20867, 20868, 20869, 20870, 20871, 20872, 20873, 20874 were selected from the ORFs having the nucleotide sequence represented by SEQ ID NO: 1355.

The probes having the nucleotide sequence represented by SEQ ID NOS: 20875, 20876, 20877, 20878, 20879, 20880, 20881, 20882, 20883, 20884, 20885 were selected from the ORFs having the nucleotide sequence represented by SEQ ID NO: 1356.

The probes having the nucleotide sequence represented by SEQ ID NOS: 20886, 20887, 20888, 20889, 20890, 20891, 20892, 20893, 20894, 20895, 20896 were selected from the ORFs having the nucleotide sequence represented by SEQ ID NO: 1357.

The probes having the nucleotide sequence represented by SEQ ID NOS: 20897, 20898, 20899, 20900, 20901, 20902, 20903, 20904, 20905, 20906, 20907 were selected from the ORFs having the nucleotide sequence represented by SEQ ID NO: 1358.

The probes having the nucleotide sequence represented by SEQ ID NOS: 20908, 20909, 20910, 20911, 20912, 20913, 20914, 20915, 20916, 20917, 20918 were selected from the ORFs having the nucleotide sequence represented by SEQ ID NO: 1359.

The probes having the nucleotide sequence represented by SEQ ID NOS: 20919, 20920, 20921, 20922, 20923, 20924, 20925, 20926, 20927, 20928, 20929 were selected from the ORFs having the nucleotide sequence represented by SEQ ID NO: 1360.

The probes having the nucleotide sequence represented by SEQ ID NOS: 20930, 20931, 20932, 20933, 20934, 20935, 20936, 20937, 20938, 20939, 20940 were selected from the ORFs having the nucleotide sequence represented by SEQ ID NO: 1361.

The probes having the nucleotide sequence represented by SEQ ID NOS: 20941, 20942, 20943, 20944, 20945, 20946, 20947, 20948, 20949, 20950, 20951 were selected from the ORFs having the nucleotide sequence represented by SEQ ID NO: 1362.

The probes having the nucleotide sequence represented by SEQ ID NOS: 20952, 20953, 20954, 20955, 20956, 20957, 20958, 20959, 20960, 20961, 20962 were selected from the ORFs having the nucleotide sequence represented by SEQ ID NO: 1363.

The probes having the nucleotide sequence represented by SEQ ID NOS: 20963, 20964, 20965, 20966, 20967, 20968, 20969, 20970, 20971, 20972, 20973 were selected from the ORFs having the nucleotide sequence represented by SEQ ID NO: 1364.

The probes having the nucleotide sequence represented by SEQ ID NOS: 20974, 20975, 20976, 20977, 20978, 20979, 20980, 20981, 20982, 20983, 20984 were selected from the ORFs having the nucleotide sequence represented by SEQ ID NO: 1365.

The probes having the nucleotide sequence represented by SEQ ID NOS: 20985, 20986, 20987, 20988, 20989, 20990, 20991, 20992, 20993, 20994, 20995 were selected from the ORFs having the nucleotide sequence represented by SEQ ID NO: 1366.

The probes having the nucleotide sequence represented by SEQ ID NOS: 20996, 20997, 20998, 20999, 21000, 21001, 21002, 21003, 21004, 21005, 21006 were selected from the ORFs having the nucleotide sequence represented by SEQ ID NO: 1367.

The probes having the nucleotide sequence represented by SEQ ID NOS: 21007, 21008, 21009, 21010, 21011, 21012, 21013, 21014, 21015, 21016, 21017 were selected from the ORFs having the nucleotide sequence represented by SEQ ID NO: 1368.

The probes having the nucleotide sequence represented by SEQ ID NOS: 21018, 21019, 21020, 21021, 21022, 21023, 21024, 21025, 21026, 21027, 21028 were selected from the ORFs having the nucleotide sequence represented by SEQ ID NO: 1369.

The probes having the nucleotide sequence represented by SEQ ID NOS: 21029, 21030, 21031, 21032, 21033, 21034, 21035, 21036, 21037, 21038, 21039 were selected from the ORFs having the nucleotide sequence represented by SEQ ID NO: 1370.

The probes having the nucleotide sequence represented by SEQ ID NOS: 21040, 21041, 21042, 21043, 21044, 21045, 21046, 21047, 21048, 21049, 21050 were selected from the ORFs having the nucleotide sequence represented by SEQ ID NO: 1371.

The probes having the nucleotide sequence represented by SEQ ID NOS: 21051, 21052, 21053, 21054, 21055, 21056, 21057, 21058, 21059, 21060, 21061 were selected from the ORFs having the nucleotide sequence represented by SEQ ID NO: 1372.

The probes having the nucleotide sequence represented by SEQ ID NOS: 21062, 21063, 21064, 21065, 21066, 21067, 21068, 21069, 21070, 21071, 21072 were selected from the ORFs having the nucleotide sequence represented by SEQ ID NO: 1373.

The probes having the nucleotide sequence represented by SEQ ID NOS: 21073, 21074, 21075, 21076, 21077, 21078, 21079, 21080, 21081, 21082, 21083 were selected from the ORFs having the nucleotide sequence represented by SEQ ID NO: 1374.

The probes having the nucleotide sequence represented by SEQ ID NOS: 21084, 21085, 21086, 21087, 21088, 21089, 21090, 21091, 21092, 21093, 21094 were selected from the ORFs having the nucleotide sequence represented by SEQ ID NO: 1375.

The probes having the nucleotide sequence represented by SEQ ID NOS: 21095, 21096, 21097, 21098, 21099, 21100, 21101, 21102, 21103, 21104, 21105 were selected from the ORFs having the nucleotide sequence represented by SEQ ID NO: 1376.

The probes having the nucleotide sequence represented by SEQ ID NOS: 21106, 21107, 21108, 21109, 21110, 21111, 21112, 21113, 21114, 21115, 21116 were selected from the ORFs having the nucleotide sequence represented by SEQ ID NO: 1377.

The probes having the nucleotide sequence represented by SEQ ID NOS: 21117, 21118, 21119, 21120, 21121, 21122, 21123, 21124, 21125, 21126, 21127 were selected from the ORFs having the nucleotide sequence represented by SEQ ID NO: 1378.

The probes having the nucleotide sequence represented by SEQ ID NOS: 21128, 21129, 21130, 21131, 21132, 21133, 21134, 21135, 21136, 21137, 21138 were selected from the ORFs having the nucleotide sequence represented by SEQ ID NO: 1379.

The probes having the nucleotide sequence represented by SEQ ID NOS: 21139, 21140, 21141, 21142, 21143, 21144, 21145, 21146, 21147, 21148, 21149 were selected from the ORFs having the nucleotide sequence represented by SEQ ID NO: 1380.

The probes having the nucleotide sequence represented by SEQ ID NOS: 21150, 21151, 21152, 21153, 21154, 21155, 21156, 21157, 21158, 21159, 21160 were selected from the ORFs having the nucleotide sequence represented by SEQ ID NO: 1381.

The probes having the nucleotide sequence represented by SEQ ID NOS: 21161, 21162, 21163, 21164, 21165, 21166, 21167, 21168, 21169, 21170, 21171 were selected from the ORFs having the nucleotide sequence represented by SEQ ID NO: 1382.

The probes having the nucleotide sequence represented by SEQ ID NOS: 21172, 21173, 21174, 21175, 21176, 21177, 21178, 21179, 21180, 21181, 21182 were selected from the ORFs having the nucleotide sequence represented by SEQ ID NO: 1383.

The probes having the nucleotide sequence represented by SEQ ID NOS: 21183, 21184, 21185, 21186, 21187, 21188, 21189, 21190, 21191, 21192, 21193, 21194, 21195, 21196, 21197, 21198, 21199, 21200, 21201, 21202, 21203, 21204 were selected from the ORFs having the nucleotide sequence represented by SEQ ID NO: 1384.

The probes having the nucleotide sequence represented by SEQ ID NOS: 21205, 21206, 21207, 21208, 21209, 21210, 21211, 21212, 21213, 21214, 21215 were selected from the ORFs having the nucleotide sequence represented by SEQ ID NO: 1385.

The probes having the nucleotide sequence represented by SEQ ID NOS: 21216, 21217, 21218, 21219, 21220, 21221, 21222, 21223, 21224, 21225, 21226 were selected from the ORFs having the nucleotide sequence represented by SEQ ID NO: 1386.

The probes having the nucleotide sequence represented by SEQ ID NOS: 21227, 21228, 21229, 21230, 21231, 21232, 21233, 21234, 21235, 21236, 21237 were selected from the ORFs having the nucleotide sequence represented by SEQ ID NO: 1387.

The probes having the nucleotide sequence represented by SEQ ID NOS: 21238, 21239, 21240, 21241, 21242, 21243, 21244, 21245, 21246, 21247, 21248 were selected from the ORFs having the nucleotide sequence represented by SEQ ID NO: 1388.

The probes having the nucleotide sequence represented by SEQ ID NOS: 21249, 21250, 21251, 21252, 21253, 21254, 21255, 21256, 21257, 21258, 21259 were selected from the ORFs having the nucleotide sequence represented by SEQ ID NO: 1389.

The probes having the nucleotide sequence represented by SEQ ID NOS: 21260, 21261, 21262, 21263, 21264, 21265, 21266, 21267, 21268, 21269, 21270 were selected from the ORFs having the nucleotide sequence represented by SEQ ID NO: 1390.

The probes having the nucleotide sequence represented by SEQ ID NOS: 21271, 21272, 21273, 21274, 21275, 21276, 21277, 21278, 21279, 21280, 21281 were selected from the ORFs having the nucleotide sequence represented by SEQ ID NO: 1391.

The probes having the nucleotide sequence represented by SEQ ID NOS: 21282, 21283, 21284, 21285, 21286, 21287, 21288, 21289, 21290, 21291, 21292 were selected from the ORFs having the nucleotide sequence represented by SEQ ID NO: 1392.

The probes having the nucleotide sequence represented by SEQ ID NOS: 21293, 21294, 21295, 21296, 21297, 21298, 21299, 21300, 21301, 21302, 21303 were selected from the ORFs having the nucleotide sequence represented by SEQ ID NO: 1393.

The probes having the nucleotide sequence represented by SEQ ID NOS: 21304, 21305, 21306, 21307, 21308, 21309, 21310, 21311, 21312, 21313, 21314 were selected from the ORFs having the nucleotide sequence represented by SEQ ID NO: 1394.

The probes having the nucleotide sequence represented by SEQ ID NOS: 21315, 21316, 21317, 21318, 21319, 21320, 21321, 21322, 21323, 21324, 21325 were selected from the ORFs having the nucleotide sequence represented by SEQ ID NO: 1395.

The probes having the nucleotide sequence represented by SEQ ID NOS: 21326, 21327, 21328, 21329, 21330, 21331, 21332, 21333, 21334, 21335, 21336 were selected from the ORFs having the nucleotide sequence represented by SEQ ID NO: 1396.

The probes having the nucleotide sequence represented by SEQ ID NOS: 21337, 21338, 21339, 21340, 21341, 21342, 21343, 21344, 21345, 21346, 21347 were selected from the ORFs having the nucleotide sequence represented by SEQ ID NO: 1397.

The probes having the nucleotide sequence represented by SEQ ID NOS: 21348, 21349, 21350, 21351, 21352, 21353, 21354, 21355, 21356, 21357, 21358 were selected from the ORFs having the nucleotide sequence represented by SEQ ID NO: 1398.

The probes having the nucleotide sequence represented by SEQ ID NOS: 21359, 21360, 21361, 21362, 21363, 21364, 21365, 21366, 21367, 21368, 21369 were selected from the ORFs having the nucleotide sequence represented by SEQ ID NO: 1399.

The probes having the nucleotide sequence represented by SEQ ID NOS: 21370, 21371, 21372, 21373, 21374, 21375, 21376, 21377, 21378, 21379, 21380 were selected from the ORFs having the nucleotide sequence represented by SEQ ID NO: 1400.

The probes having the nucleotide sequence represented by SEQ ID NOS: 21381, 21382, 21383, 21384, 21385, 21386, 21387, 21388, 21389, 21390, 21391 were selected from the ORFs having the nucleotide sequence represented by SEQ ID NO: 1401.

The probes having the nucleotide sequence represented by SEQ ID NOS: 21392, 21393, 21394, 21395, 21396, 21397, 21398, 21399, 21400, 21401, 21402 were selected from the ORFs having the nucleotide sequence represented by SEQ ID NO: 1402.

The probes having the nucleotide sequence represented by SEQ ID NOS: 21403, 21404, 21405, 21406, 21407, 21408, 21409, 21410, 21411, 21412, 21413 were selected from the ORFs having the nucleotide sequence represented by SEQ ID NO: 1403.

The probes having the nucleotide sequence represented by SEQ ID NOS: 21414, 21415, 21416, 21417, 21418, 21419, 21420, 21421, 21422, 21423, 21424 were selected from the ORFs having the nucleotide sequence represented by SEQ ID NO: 1404.

The probes having the nucleotide sequence represented by SEQ ID NOS: 21425, 21426, 21427, 21428, 21429, 21430, 21431, 21432, 21433, 21434, 21435 were selected from the ORFs having the nucleotide sequence represented by SEQ ID NO: 1405.

The probes having the nucleotide sequence represented by SEQ ID NOS: 21436, 21437, 21438, 21439, 21440, 21441, 21442, 21443, 21444, 21445, 21446 were selected from the ORFs having the nucleotide sequence represented by SEQ ID NO: 1406.

The probes having the nucleotide sequence represented by SEQ ID NOS: 21447, 21448, 21449, 21450, 21451, 21452, 21453, 21454, 21455, 21456, 21457 were selected from the ORFs having the nucleotide sequence represented by SEQ ID NO: 1407.

The probes having the nucleotide sequence represented by SEQ ID NOS: 21458, 21459, 21460, 21461, 21462, 21463, 21464, 21465, 21466, 21467, 21468 were selected from the ORFs having the nucleotide sequence represented by SEQ ID NO: 1408.

The probes having the nucleotide sequence represented by SEQ ID NOS: 21469, 21470, 21471, 21472, 21473, 21474, 21475, 21476, 21477, 21478, 21479 were selected from the ORFs having the nucleotide sequence represented by SEQ ID NO: 1409.

The probes having the nucleotide sequence represented by SEQ ID NOS: 21480, 21481, 21482, 21483, 21484, 21485, 21486, 21487, 21488, 21489, 21490 were selected from the ORFs having the nucleotide sequence represented by SEQ ID NO: 1410.

The probes having the nucleotide sequence represented by SEQ ID NOS: 21491, 21492, 21493, 21494, 21495, 21496, 21497, 21498, 21499, 21500, 21501 were selected from the ORFs having the nucleotide sequence represented by SEQ ID NO: 1411.

The probes having the nucleotide sequence represented by SEQ ID NOS: 21502, 21503, 21504, 21505, 21506, 21507, 21508, 21509, 21510, 21511, 21512 were selected from the ORFs having the nucleotide sequence represented by SEQ ID NO: 1412.

The probes having the nucleotide sequence represented by SEQ ID NOS: 21513, 21514, 21515, 21516, 21517, 21518, 21519, 21520, 21521, 21522, 21523 were selected from the ORFs having the nucleotide sequence represented by SEQ ID NO: 1413.

The probes having the nucleotide sequence represented by SEQ ID NOS: 21524, 21525, 21526, 21527, 21528, 21529, 21530, 21531, 21532, 21533, 21534 were selected from the ORFs having the nucleotide sequence represented by SEQ ID NO: 1414.

The probes having the nucleotide sequence represented by SEQ ID NOS: 21535, 21536, 21537, 21538, 21539, 21540, 21541, 21542, 21543, 21544, 21545 were selected from the ORFs having the nucleotide sequence represented by SEQ ID NO: 1415.

The probes having the nucleotide sequence represented by SEQ ID NOS: 21546, 21547, 21548, 21549, 21550, 21551, 21552, 21553, 21554, 21555, 21556 were selected from the ORFs having the nucleotide sequence represented by SEQ ID NO: 1416.

The probes having the nucleotide sequence represented by SEQ ID NOS: 21557, 21558, 21559, 21560, 21561, 21562, 21563, 21564, 21565, 21566, 21567 were selected from the ORFs having the nucleotide sequence represented by SEQ ID NO: 1417.

The probes having the nucleotide sequence represented by SEQ ID NOS: 21568, 21569, 21570, 21571, 21572, 21573, 21574, 21575, 21576, 21577, 21578 were selected from the ORFs having the nucleotide sequence represented by SEQ ID NO: 1418.

The probes having the nucleotide sequence represented by SEQ ID NOS: 21579, 21580, 21581, 21582, 21583, 21584, 21585, 21586, 21587, 21588, 21589 were selected from the ORFs having the nucleotide sequence represented by SEQ ID NO: 1419.

The probes having the nucleotide sequence represented by SEQ ID NOS: 21590, 21591, 21592, 21593, 21594, 21595, 21596, 21597, 21598, 21599, 21600 were selected from the ORFs having the nucleotide sequence represented by SEQ ID NO: 1420.

The probes having the nucleotide sequence represented by SEQ ID NOS: 21601, 21602, 21603, 21604, 21605, 21606, 21607, 21608, 21609, 21610, 21611 were selected from the ORFs having the nucleotide sequence represented by SEQ ID NO: 1421.

The probes having the nucleotide sequence represented by SEQ ID NOS: 21612, 21613, 21614, 21615, 21616, 21617, 21618, 21619, 21620, 21621, 21622 were selected from the ORFs having the nucleotide sequence represented by SEQ ID NO: 1422.

The probes having the nucleotide sequence represented by SEQ ID NOS: 21623, 21624, 21625, 21626, 21627, 21628, 21629, 21630, 21631, 21632, 21633 were selected from the ORFs having the nucleotide sequence represented by SEQ ID NO: 1423.

The probes having the nucleotide sequence represented by SEQ ID NOS: 21634, 21635, 21636, 21637, 21638, 21639, 21640, 21641, 21642, 21643, 21644 were selected from the ORFs having the nucleotide sequence represented by SEQ ID NO: 1424.

The probes having the nucleotide sequence represented by SEQ ID NOS: 21645, 21646, 21647, 21648, 21649, 21650, 21651, 21652, 21653, 21654, 21655 were selected from the ORFs having the nucleotide sequence represented by SEQ ID NO: 1425.

The probes having the nucleotide sequence represented by SEQ ID NOS: 21656, 21657, 21658, 21659, 21660, 21661, 21662, 21663, 21664, 21665, 21666 were selected from the ORFs having the nucleotide sequence represented by SEQ ID NO: 1426.

The probes having the nucleotide sequence represented by SEQ ID NOS: 21667, 21668, 21669, 21670, 21671, 21672, 21673, 21674, 21675, 21676, 21677 were selected from the ORFs having the nucleotide sequence represented by SEQ ID NO: 1427.

The probes having the nucleotide sequence represented by SEQ ID NOS: 21678, 21679, 21680, 21681, 21682, 21683, 21684, 21685, 21686, 21687, 21688 were selected from the ORFs having the nucleotide sequence represented by SEQ ID NO: 1428.

The probes having the nucleotide sequence represented by SEQ ID NOS: 21689, 21690, 21691, 21692, 21693, 21694, 21695, 21696, 21697, 21698, 21699 were selected from the ORFs having the nucleotide sequence represented by SEQ ID NO: 1429.

The probes having the nucleotide sequence represented by SEQ ID NOS: 21700, 21701, 21702, 21703, 21704, 21705, 21706, 21707, 21708, 21709, 21710 were selected from the ORFs having the nucleotide sequence represented by SEQ ID NO: 1430.

The probes having the nucleotide sequence represented by SEQ ID NOS: 21711, 21712, 21713, 21714, 21715, 21716, 21717, 21718, 21719, 21720, 21721 were selected from the ORFs having the nucleotide sequence represented by SEQ ID NO: 1431.

The probes having the nucleotide sequence represented by SEQ ID NOS: 21722, 21723, 21724, 21725, 21726, 21727, 21728, 21729, 21730, 21731, 21732 were selected from the ORFs having the nucleotide sequence represented by SEQ ID NO: 1432.

The probes having the nucleotide sequence represented by SEQ ID NOS: 21733, 21734, 21735, 21736, 21737, 21738, 21739, 21740, 21741, 21742, 21743 were selected from the ORFs having the nucleotide sequence represented by SEQ ID NO: 1433.

The probes having the nucleotide sequence represented by SEQ ID NOS: 21744, 21745, 21746, 21747, 21748, 21749, 21750, 21751, 21752, 21753, 21754 were selected from the ORFs having the nucleotide sequence represented by SEQ ID NO: 1434.

The probes having the nucleotide sequence represented by SEQ ID NOS: 21755, 21756, 21757, 21758, 21759, 21760, 21761, 21762, 21763, 21764, 21765 were selected from the ORFs having the nucleotide sequence represented by SEQ ID NO: 1435.

The probes having the nucleotide sequence represented by SEQ ID NOS: 21766, 21767, 21768, 21769, 21770, 21771, 21772, 21773, 21774, 21775, 21776 were selected from the ORFs having the nucleotide sequence represented by SEQ ID NO: 1436.

The probes having the nucleotide sequence represented by SEQ ID NOS: 21777, 21778, 21779, 21780, 21781, 21782, 21783, 21784, 21785, 21786, 21787 were selected from the ORFs having the nucleotide sequence represented by SEQ ID NO: 1437.

The probes having the nucleotide sequence represented by SEQ ID NOS: 21788, 21789, 21790, 21791, 21792, 21793, 21794, 21795, 21796, 21797, 21798 were selected from the ORFs having the nucleotide sequence represented by SEQ ID NO: 1438.

The probes having the nucleotide sequence represented by SEQ ID NOS: 21799, 21800, 21801, 21802, 21803, 21804, 21805, 21806, 21807, 21808, 21809 were selected from the ORFs having the nucleotide sequence represented by SEQ ID NO: 1439.

The probes having the nucleotide sequence represented by SEQ ID NOS: 21810, 21811, 21812, 21813, 21814, 21815, 21816, 21817, 21818, 21819, 21820 were selected from the ORFs having the nucleotide sequence represented by SEQ ID NO: 1440.

The probes having the nucleotide sequence represented by SEQ ID NOS: 21821, 21822, 21823, 21824, 21825, 21826, 21827, 21828, 21829, 21830, 21831 were selected from the ORFs having the nucleotide sequence represented by SEQ ID NO: 1441.

The probes having the nucleotide sequence represented by SEQ ID NOS: 21832, 21833, 21834, 21835, 21836, 21837, 21838, 21839, 21840, 21841, 21842 were selected from the ORFs having the nucleotide sequence represented by SEQ ID NO: 1442.

The probes having the nucleotide sequence represented by SEQ ID NOS: 21843, 21844, 21845, 21846, 21847, 21848, 21849, 21850, 21851, 21852, 21853 were selected from the ORFs having the nucleotide sequence represented by SEQ ID NO: 1443.

The probes having the nucleotide sequence represented by SEQ ID NOS: 21854, 21855, 21856, 21857, 21858, 21859, 21860, 21861, 21862, 21863, 21864 were selected from the ORFs having the nucleotide sequence represented by SEQ ID NO: 1444.

The probes having the nucleotide sequence represented by SEQ ID NOS: 21865, 21866, 21867, 21868, 21869, 21870, 21871, 21872, 21873, 21874, 21875 were selected from the ORFs having the nucleotide sequence represented by SEQ ID NO: 1445.

The probes having the nucleotide sequence represented by SEQ ID NOS: 21876, 21877, 21878, 21879, 21880, 21881, 21882, 21883, 21884, 21885, 21886 were selected from the ORFs having the nucleotide sequence represented by SEQ ID NO: 1446.

The probes having the nucleotide sequence represented by SEQ ID NOS: 21887, 21888, 21889, 21890, 21891, 21892, 21893, 21894, 21895, 21896, 21897 were selected from the ORFs having the nucleotide sequence represented by SEQ ID NO: 1447.

The probes having the nucleotide sequence represented by SEQ ID NOS: 21898, 21899, 21900, 21901, 21902, 21903, 21904, 21905, 21906, 21907, 21908 were selected from the ORFs having the nucleotide sequence represented by SEQ ID NO: 1448.

The probes having the nucleotide sequence represented by SEQ ID NOS: 21909, 21910, 21911, 21912, 21913, 21914, 21915, 21916, 21917, 21918, 21919 were selected from the ORFs having the nucleotide sequence represented by SEQ ID NO: 1449.

The probes having the nucleotide sequence represented by SEQ ID NOS: 21920, 21921, 21922, 21923, 21924, 21925, 21926, 21927, 21928, 21929, 21930 were selected from the ORFs having the nucleotide sequence represented by SEQ ID NO: 1450.

The probes having the nucleotide sequence represented by SEQ ID NOS: 21931, 21932, 21933, 21934, 21935, 21936, 21937, 21938, 21939, 21940, 21941 were selected from the ORFs having the nucleotide sequence represented by SEQ ID NO: 1451.

The probes having the nucleotide sequence represented by SEQ ID NOS: 21942, 21943, 21944, 21945, 21946, 21947, 21948, 21949, 21950, 21951, 21952 were selected from the ORFs having the nucleotide sequence represented by SEQ ID NO: 1452.

The probes having the nucleotide sequence represented by SEQ ID NOS: 21953, 21954, 21955, 21956, 21957, 21958, 21959, 21960, 21961, 21962, 21963 were selected from the ORFs having the nucleotide sequence represented by SEQ ID NO: 1453.

The probes having the nucleotide sequence represented by SEQ ID NOS: 21964, 21965, 21966, 21967, 21968, 21969, 21970, 21971, 21972, 21973, 21974 were selected from the ORFs having the nucleotide sequence represented by SEQ ID NO: 1454.

The probes having the nucleotide sequence represented by SEQ ID NOS: 21975, 21976, 21977, 21978, 21979, 21980, 21981, 21982, 21983, 21984, 21985 were selected from the ORFs having the nucleotide sequence represented by SEQ ID NO: 1455.

The probes having the nucleotide sequence represented by SEQ ID NOS: 21986, 21987, 21988, 21989, 21990, 21991, 21992, 21993, 21994, 21995, 21996 were selected from the ORFs having the nucleotide sequence represented by SEQ ID NO: 1456.

The probes having the nucleotide sequence represented by SEQ ID NOS: 21997, 21998, 21999, 22000, 22001, 22002, 22003, 22004, 22005, 22006, 22007 were selected from the ORFs having the nucleotide sequence represented by SEQ ID NO: 1457.

The probes having the nucleotide sequence represented by SEQ ID NOS: 22008, 22009, 22010, 22011, 22012, 22013, 22014, 22015, 22016, 22017, 22018 were selected from the ORFs having the nucleotide sequence represented by SEQ ID NO: 1458.

The probes having the nucleotide sequence represented by SEQ ID NOS: 22019, 22020, 22021, 22022, 22023, 22024, 22025, 22026, 22027, 22028, 22029 were selected from the ORFs having the nucleotide sequence represented by SEQ ID NO: 1459.

The probes having the nucleotide sequence represented by SEQ ID NOS: 22030, 22031, 22032, 22033, 22034, 22035, 22036, 22037, 22038, 22039, 22040 were selected from the ORFs having the nucleotide sequence represented by SEQ ID NO: 1460.

The probes having the nucleotide sequence represented by SEQ ID NOS: 22041, 22042, 22043, 22044, 22045, 22046, 22047, 22048, 22049, 22050, 22051 were selected from the ORFs having the nucleotide sequence represented by SEQ ID NO: 1461.

The probes having the nucleotide sequence represented by SEQ ID NOS: 22052, 22053, 22054, 22055, 22056, 22057, 22058, 22059, 22060, 22061, 22062 were selected from the ORFs having the nucleotide sequence represented by SEQ ID NO: 1462.

The probes having the nucleotide sequence represented by SEQ ID NOS: 22063, 22064, 22065, 22066, 22067, 22068, 22069, 22070, 22071, 22072, 22073 were selected from the ORFs having the nucleotide sequence represented by SEQ ID NO: 1463.

The probes having the nucleotide sequence represented by SEQ ID NOS: 22074, 22075, 22076, 22077, 22078, 22079, 22080, 22081, 22082, 22083, 22084 were selected from the ORFs having the nucleotide sequence represented by SEQ ID NO: 1464.

The probes having the nucleotide sequence represented by SEQ ID NOS: 22085, 22086, 22087, 22088, 22089, 22090, 22091, 22092, 22093, 22094, 22095 were selected from the ORFs having the nucleotide sequence represented by SEQ ID NO: 1465.

The probes having the nucleotide sequence represented by SEQ ID NOS: 22096, 22097, 22098, 22099, 22100, 22101, 22102, 22103, 22104, 22105, 22106 were selected from the ORFs having the nucleotide sequence represented by SEQ ID NO: 1466.

The probes having the nucleotide sequence represented by SEQ ID NOS: 22107, 22108, 22109, 22110, 22111, 22112, 22113, 22114, 22115, 22116, 22117 were selected from the ORFs having the nucleotide sequence represented by SEQ ID NO: 1467.

The probes having the nucleotide sequence represented by SEQ ID NOS: 22118, 22119, 22120, 22121, 22122, 22123, 22124, 22125, 22126, 22127, 22128 were selected from the ORFs having the nucleotide sequence represented by SEQ ID NO: 1468.

The probes having the nucleotide sequence represented by SEQ ID NOS: 22129, 22130, 22131, 22132, 22133, 22134, 22135, 22136, 22137, 22138, 22139 were selected from the ORFs having the nucleotide sequence represented by SEQ ID NO: 1469.

The probes having the nucleotide sequence represented by SEQ ID NOS: 22140, 22141, 22142, 22143, 22144, 22145, 22146, 22147, 22148, 22149, 22150 were selected from the ORFs having the nucleotide sequence represented by SEQ ID NO: 1470.

The probes having the nucleotide sequence represented by SEQ ID NOS: 22151, 22152, 22153, 22154, 22155, 22156, 22157, 22158, 22159, 22160, 22161 were selected from the ORFs having the nucleotide sequence represented by SEQ ID NO: 1471.

The probes having the nucleotide sequence represented by SEQ ID NOS: 22162, 22163, 22164, 22165, 22166, 22167, 22168, 22169, 22170, 22171, 22172 were selected from the ORFs having the nucleotide sequence represented by SEQ ID NO: 1472.

The probes having the nucleotide sequence represented by SEQ ID NOS: 22173, 22174, 22175, 22176, 22177, 22178, 22179, 22180, 22181, 22182, 22183 were selected from the ORFs having the nucleotide sequence represented by SEQ ID NO: 1473.

The probes having the nucleotide sequence represented by SEQ ID NOS: 22184, 22185, 22186, 22187, 22188, 22189, 22190, 22191, 22192, 22193, 22194 were selected from the ORFs having the nucleotide sequence represented by SEQ ID NO: 1474.

The probes having the nucleotide sequence represented by SEQ ID NOS: 22195, 22196, 22197, 22198, 22199, 22200, 22201, 22202, 22203, 22204, 22205 were selected from the ORFs having the nucleotide sequence represented by SEQ ID NO: 1475.

The probes having the nucleotide sequence represented by SEQ ID NOS: 22206, 22207, 22208, 22209, 22210, 22211, 22212, 22213, 22214, 22215, 22216 were selected from the ORFs having the nucleotide sequence represented by SEQ ID NO: 1476.

The probes having the nucleotide sequence represented by SEQ ID NOS: 22217, 22218, 22219, 22220, 22221, 22222, 22223, 22224, 22225, 22226, 22227 were selected from the ORFs having the nucleotide sequence represented by SEQ ID NO: 1477.

The probes having the nucleotide sequence represented by SEQ ID NOS: 22228, 22229, 22230, 22231, 22232, 22233, 22234, 22235, 22236, 22237, 22238 were selected from the ORFs having the nucleotide sequence represented by SEQ ID NO: 1478.

The probes having the nucleotide sequence represented by SEQ ID NOS: 22239, 22240, 22241, 22242, 22243, 22244, 22245, 22246, 22247, 22248, 22249 were selected from the ORFs having the nucleotide sequence represented by SEQ ID NO: 1479.

The probes having the nucleotide sequence represented by SEQ ID NOS: 22250, 22251, 22252, 22253, 22254, 22255, 22256, 22257, 22258, 22259, 22260 were selected from the ORFs having the nucleotide sequence represented by SEQ ID NO: 1480.

The probes having the nucleotide sequence represented by SEQ ID NOS: 22261, 22262, 22263, 22264, 22265, 22266, 22267, 22268, 22269, 22270, 22271 were selected from the ORFs having the nucleotide sequence represented by SEQ ID NO: 1481.

The probes having the nucleotide sequence represented by SEQ ID NOS: 22272, 22273, 22274, 22275, 22276, 22277, 22278, 22279, 22280, 22281, 22282 were selected from the ORFs having the nucleotide sequence represented by SEQ ID NO: 1482.

The probes having the nucleotide sequence represented by SEQ ID NOS: 22283, 22284, 22285, 22286, 22287, 22288, 22289, 22290, 22291, 22292, 22293 were selected from the ORFs having the nucleotide sequence represented by SEQ ID NO: 1483.

The probes having the nucleotide sequence represented by SEQ ID NOS: 22294, 22295, 22296, 22297, 22298, 22299, 22300, 22301, 22302, 22303, 22304 were selected from the ORFs having the nucleotide sequence represented by SEQ ID NO: 1484.

The probes having the nucleotide sequence represented by SEQ ID NOS: 22305, 22306, 22307, 22308, 22309, 22310, 22311, 22312, 22313, 22314, 22315 were selected from the ORFs having the nucleotide sequence represented by SEQ ID NO: 1485.

The probes having the nucleotide sequence represented by SEQ ID NOS: 22316, 22317, 22318, 22319, 22320, 22321, 22322, 22323, 22324, 22325, 22326 were selected from the ORFs having the nucleotide sequence represented by SEQ ID NO: 1486.

The probes having the nucleotide sequence represented by SEQ ID NOS: 22327, 22328, 22329, 22330, 22331, 22332, 22333, 22334, 22335, 22336, 22337 were selected from the ORFs having the nucleotide sequence represented by SEQ ID NO: 1487.

The probes having the nucleotide sequence represented by SEQ ID NOS: 22338, 22339, 22340, 22341, 22342, 22343, 22344, 22345, 22346, 22347, 22348 were selected from the ORFs having the nucleotide sequence represented by SEQ ID NO: 1488.

The probes having the nucleotide sequence represented by SEQ ID NOS: 22349, 22350, 22351, 22352, 22353, 22354, 22355, 22356, 22357, 22358, 22359 were selected from the ORFs having the nucleotide sequence represented by SEQ ID NO: 1489.

The probes having the nucleotide sequence represented by SEQ ID NOS: 22360, 22361, 22362, 22363, 22364, 22365, 22366, 22367, 22368, 22369, 22370 were selected from the ORFs having the nucleotide sequence represented by SEQ ID NO: 1490.

The probes having the nucleotide sequence represented by SEQ ID NOS: 22371, 22372, 22373, 22374, 22375, 22376, 22377, 22378, 22379, 22380, 22381 were selected from the ORFs having the nucleotide sequence represented by SEQ ID NO: 1491.

The probes having the nucleotide sequence represented by SEQ ID NOS: 22382, 22383, 22384, 22385, 22386, 22387, 22388, 22389, 22390, 22391, 22392 were selected from the ORFs having the nucleotide sequence represented by SEQ ID NO: 1492.

The probes having the nucleotide sequence represented by SEQ ID NOS: 22393, 22394, 22395, 22396, 22397, 22398, 22399, 22400, 22401, 22402, 22403 were selected from the ORFs having the nucleotide sequence represented by SEQ ID NO: 1493.

The probes having the nucleotide sequence represented by SEQ ID NOS: 22404, 22405, 22406, 22407, 22408, 22409, 22410, 22411, 22412, 22413, 22414 were selected from the ORFs having the nucleotide sequence represented by SEQ ID NO: 1494.

The probes having the nucleotide sequence represented by SEQ ID NOS: 22415, 22416, 22417, 22418, 22419, 22420, 22421, 22422, 22423, 22424, 22425 were selected from the ORFs having the nucleotide sequence represented by SEQ ID NO: 1495.

The probes having the nucleotide sequence represented by SEQ ID NOS: 22426, 22427, 22428, 22429, 22430, 22431, 22432, 22433, 22434, 22435, 22436 were selected from the ORFs having the nucleotide sequence represented by SEQ ID NO: 1496.

The probes having the nucleotide sequence represented by SEQ ID NOS: 22437, 22438, 22439, 22440, 22441, 22442, 22443, 22444, 22445, 22446, 22447 were selected from the ORFs having the nucleotide sequence represented by SEQ ID NO: 1497.

The probes having the nucleotide sequence represented by SEQ ID NOS: 22448, 22449, 22450, 22451, 22452, 22453, 22454, 22455, 22456, 22457, 22458 were selected from the ORFs having the nucleotide sequence represented by SEQ ID NO: 1498.

The probes having the nucleotide sequence represented by SEQ ID NOS: 22459, 22460, 22461, 22462, 22463, 22464, 22465, 22466, 22467, 22468, 22469 were selected from the ORFs having the nucleotide sequence represented by SEQ ID NO: 1499.

The probes having the nucleotide sequence represented by SEQ ID NOS: 22470, 22471, 22472, 22473, 22474, 22475, 22476, 22477, 22478, 22479, 22480 were selected from the ORFs having the nucleotide sequence represented by SEQ ID NO: 1500.

The probes having the nucleotide sequence represented by SEQ ID NOS: 22481, 22482, 22483, 22484, 22485, 22486, 22487, 22488, 22489, 22490, 22491 were selected from the ORFs having the nucleotide sequence represented by SEQ ID NO: 1501.

The probes having the nucleotide sequence represented by SEQ ID NOS: 22492, 22493, 22494, 22495, 22496, 22497, 22498, 22499, 22500, 22501, 22502 were selected from the ORFs having the nucleotide sequence represented by SEQ ID NO: 1502.

The probes having the nucleotide sequence represented by SEQ ID NOS: 22503, 22504, 22505, 22506, 22507, 22508, 22509, 22510, 22511, 22512, 22513 were selected from the ORFs having the nucleotide sequence represented by SEQ ID NO: 1503.

The probes having the nucleotide sequence represented by SEQ ID NOS: 22514, 22515, 22516, 22517, 22518, 22519, 22520, 22521, 22522, 22523, 22524 were selected from the ORFs having the nucleotide sequence represented by SEQ ID NO: 1504.

The probes having the nucleotide sequence represented by SEQ ID NOS: 22525, 22526, 22527, 22528, 22529, 22530, 22531, 22532, 22533, 22534, 22535 were selected from the ORFs having the nucleotide sequence represented by SEQ ID NO: 1505.

The probes having the nucleotide sequence represented by SEQ ID NOS: 22536, 22537, 22538, 22539, 22540, 22541, 22542, 22543, 22544, 22545, 22546 were selected from the ORFs having the nucleotide sequence represented by SEQ ID NO: 1506.

The probes having the nucleotide sequence represented by SEQ ID NOS: 22547, 22548, 22549, 22550, 22551, 22552, 22553, 22554, 22555, 22556, 22557 were selected from the ORFs having the nucleotide sequence represented by SEQ ID NO: 1507.

The probes having the nucleotide sequence represented by SEQ ID NOS: 22558, 22559, 22560, 22561, 22562, 22563, 22564, 22565, 22566, 22567, 22568 were selected from the ORFs having the nucleotide sequence represented by SEQ ID NO: 1508.

The probes having the nucleotide sequence represented by SEQ ID NOS: 22569, 22570, 22571, 22572, 22573, 22574, 22575, 22576, 22577, 22578, 22579 were selected from the ORFs having the nucleotide sequence represented by SEQ ID NO: 1509.

The probes having the nucleotide sequence represented by SEQ ID NOS: 22580, 22581, 22582, 22583, 22584, 22585, 22586, 22587, 22588, 22589, 22590 were selected from the ORFs having the nucleotide sequence represented by SEQ ID NO: 1510.

The probes having the nucleotide sequence represented by SEQ ID NOS: 22591, 22592, 22593, 22594, 22595, 22596, 22597, 22598, 22599, 22600, 22601 were selected from the ORFs having the nucleotide sequence represented by SEQ ID NO: 1511.

The probes having the nucleotide sequence represented by SEQ ID NOS: 22602, 22603, 22604, 22605, 22606, 22607, 22608, 22609, 22610, 22611, 22612 were selected from the ORFs having the nucleotide sequence represented by SEQ ID NO: 1512.

The probes having the nucleotide sequence represented by SEQ ID NOS: 22613, 22614, 22615, 22616, 22617, 22618, 22619, 22620, 22621, 22622, 22623 were selected from the ORFs having the nucleotide sequence represented by SEQ ID NO: 1513.

The probes having the nucleotide sequence represented by SEQ ID NOS: 22624, 22625, 22626, 22627, 22628, 22629, 22630, 22631, 22632, 22633, 22634 were selected from the ORFs having the nucleotide sequence represented by SEQ ID NO: 1514.

The probes having the nucleotide sequence represented by SEQ ID NOS: 22635, 22636, 22637, 22638, 22639, 22640, 22641, 22642, 22643, 22644, 22645 were selected from the ORFs having the nucleotide sequence represented by SEQ ID NO: 1515.

The probes having the nucleotide sequence represented by SEQ ID NOS: 22646, 22647, 22648, 22649, 22650, 22651, 22652, 22653, 22654, 22655, 22656 were selected from the ORFs having the nucleotide sequence represented by SEQ ID NO: 1516.

The probes having the nucleotide sequence represented by SEQ ID NOS: 22657, 22658, 22659, 22660, 22661, 22662, 22663, 22664, 22665, 22666, 22667 were selected from the ORFs having the nucleotide sequence represented by SEQ ID NO: 1517.

The probes having the nucleotide sequence represented by SEQ ID NOS: 22668, 22669, 22670, 22671, 22672, 22673, 22674, 22675, 22676, 22677, 22678 were selected from the ORFs having the nucleotide sequence represented by SEQ ID NO: 1518.

The probes having the nucleotide sequence represented by SEQ ID NOS: 22679, 22680, 22681, 22682, 22683, 22684, 22685, 22686, 22687, 22688, 22689 were selected from the ORFs having the nucleotide sequence represented by SEQ ID NO: 1519.

The probes having the nucleotide sequence represented by SEQ ID NOS: 22690, 22691, 22692, 22693, 22694, 22695, 22696, 22697, 22698, 22699, 22700 were selected from the ORFs having the nucleotide sequence represented by SEQ ID NO: 1520.

The probes having the nucleotide sequence represented by SEQ ID NOS: 22701, 22702, 22703, 22704, 22705, 22706, 22707, 22708, 22709, 22710, 22711 were selected from the ORFs having the nucleotide sequence represented by SEQ ID NO: 1521.

The probes having the nucleotide sequence represented by SEQ ID NOS: 22712, 22713, 22714, 22715, 22716, 22717, 22718, 22719, 22720, 22721, 22722 were selected from the ORFs having the nucleotide sequence represented by SEQ ID NO: 1522.

The probes having the nucleotide sequence represented by SEQ ID NOS: 22723, 22724, 22725, 22726, 22727, 22728, 22729, 22730, 22731, 22732, 22733 were selected from the ORFs having the nucleotide sequence represented by SEQ ID NO: 1523.

The probes having the nucleotide sequence represented by SEQ ID NOS: 22734, 22735, 22736, 22737, 22738, 22739, 22740, 22741, 22742, 22743, 22744 were selected from the ORFs having the nucleotide sequence represented by SEQ ID NO: 1524.

The probes having the nucleotide sequence represented by SEQ ID NOS: 22745, 22746, 22747, 22748, 22749, 22750, 22751, 22752, 22753, 22754, 22755 were selected from the ORFs having the nucleotide sequence represented by SEQ ID NO: 1525.

The probes having the nucleotide sequence represented by SEQ ID NOS: 22756, 22757, 22758, 22759, 22760, 22761, 22762, 22763, 22764, 22765, 22766 were selected from the ORFs having the nucleotide sequence represented by SEQ ID NO: 1526.

The probes having the nucleotide sequence represented by SEQ ID NOS: 22767, 22768, 22769, 22770, 22771, 22772, 22773, 22774, 22775, 22776, 22777 were selected from the ORFs having the nucleotide sequence represented by SEQ ID NO: 1527.

The probes having the nucleotide sequence represented by SEQ ID NOS: 22778, 22779, 22780, 22781, 22782, 22783, 22784, 22785, 22786, 22787, 22788 were selected from the ORFs having the nucleotide sequence represented by SEQ ID NO: 1528.

The probes having the nucleotide sequence represented by SEQ ID NOS: 22789, 22790, 22791, 22792, 22793, 22794, 22795, 22796, 22797, 22798, 22799 were selected from the ORFs having the nucleotide sequence represented by SEQ ID NO: 1529.

The probes having the nucleotide sequence represented by SEQ ID NOS: 22800, 22801, 22802, 22803, 22804, 22805, 22806, 22807, 22808, 22809, 22810 were selected from the ORFs having the nucleotide sequence represented by SEQ ID NO: 1530.

The probes having the nucleotide sequence represented by SEQ ID NOS: 22811, 22812, 22813, 22814, 22815, 22816, 22817, 22818, 22819, 22820, 22821 were selected from the ORFs having the nucleotide sequence represented by SEQ ID NO: 1531.

The probes having the nucleotide sequence represented by SEQ ID NOS: 22822, 22823, 22824, 22825, 22826, 22827, 22828, 22829, 22830, 22831, 22832 were selected from the ORFs having the nucleotide sequence represented by SEQ ID NO: 1532.

The probes having the nucleotide sequence represented by SEQ ID NOS: 22833, 22834, 22835, 22836, 22837, 22838, 22839, 22840, 22841, 22842, 22843 were selected from the ORFs having the nucleotide sequence represented by SEQ ID NO: 1533.

The probes having the nucleotide sequence represented by SEQ ID NOS: 22844, 22845, 22846, 22847, 22848, 22849, 22850, 22851, 22852, 22853, 22854 were selected from the ORFs having the nucleotide sequence represented by SEQ ID NO: 1534.

The probes having the nucleotide sequence represented by SEQ ID NOS: 22855, 22856, 22857, 22858, 22859, 22860, 22861, 22862, 22863, 22864, 22865 were selected from the ORFs having the nucleotide sequence represented by SEQ ID NO: 1535.

The probes having the nucleotide sequence represented by SEQ ID NOS: 22866, 22867, 22868, 22869, 22870, 22871, 22872, 22873, 22874, 22875, 22876 were selected from the ORFs having the nucleotide sequence represented by SEQ ID NO: 1536.

The probes having the nucleotide sequence represented by SEQ ID NOS: 22877, 22878, 22879, 22880, 22881, 22882, 22883, 22884, 22885, 22886, 22887 were selected from the ORFs having the nucleotide sequence represented by SEQ ID NO: 1537.

The probes having the nucleotide sequence represented by SEQ ID NOS: 22888, 22889, 22890, 22891, 22892, 22893, 22894, 22895, 22896, 22897, 22898 were selected from the ORFs having the nucleotide sequence represented by SEQ ID NO: 1538.

The probes having the nucleotide sequence represented by SEQ ID NOS: 22899, 22900, 22901, 22902, 22903, 22904, 22905, 22906, 22907, 22908, 22909 were selected from the ORFs having the nucleotide sequence represented by SEQ ID NO: 1539.

The probes having the nucleotide sequence represented by SEQ ID NOS: 22910, 22911, 22912, 22913, 22914, 22915, 22916, 22917, 22918, 22919, 22920 were selected from the ORFs having the nucleotide sequence represented by SEQ ID NO: 1540.

The probes having the nucleotide sequence represented by SEQ ID NOS: 22921, 22922, 22923, 22924, 22925, 22926, 22927, 22928, 22929, 22930, 22931 were selected from the ORFs having the nucleotide sequence represented by SEQ ID NO: 1541.

The probes having the nucleotide sequence represented by SEQ ID NOS: 22932, 22933, 22934, 22935, 22936, 22937, 22938, 22939, 22940, 22941, 22942 were selected from the ORFs having the nucleotide sequence represented by SEQ ID NO: 1542.

The probes having the nucleotide sequence represented by SEQ ID NOS: 22943, 22944, 22945, 22946, 22947, 22948, 22949, 22950, 22951, 22952, 22953 were selected from the ORFs having the nucleotide sequence represented by SEQ ID NO: 1543.

The probes having the nucleotide sequence represented by SEQ ID NOS: 22954, 22955, 22956, 22957, 22958, 22959, 22960, 22961, 22962, 22963, 22964 were selected from the ORFs having the nucleotide sequence represented by SEQ ID NO: 1544.

The probes having the nucleotide sequence represented by SEQ ID NOS: 22965, 22966, 22967, 22968, 22969, 22970, 22971, 22972, 22973, 22974, 22975 were selected from the ORFs having the nucleotide sequence represented by SEQ ID NO: 1545.

The probes having the nucleotide sequence represented by SEQ ID NOS: 22976, 22977, 22978, 22979, 22980, 22981, 22982, 22983, 22984, 22985, 22986 were selected from the ORFs having the nucleotide sequence represented by SEQ ID NO: 1546.

The probes having the nucleotide sequence represented by SEQ ID NOS: 22987, 22988, 22989, 22990, 22991, 22992, 22993, 22994, 22995, 22996, 22997 were selected from the ORFs having the nucleotide sequence represented by SEQ ID NO: 1547.

The probes having the nucleotide sequence represented by SEQ ID NOS: 22998, 22999, 23000, 23001, 23002, 23003, 23004, 23005, 23006, 23007, 23008 were selected from the ORFs having the nucleotide sequence represented by SEQ ID NO: 1548.

The probes having the nucleotide sequence represented by SEQ ID NOS: 23009, 23010, 23011, 23012, 23013, 23014, 23015, 23016, 23017, 23018, 23019 were selected from the ORFs having the nucleotide sequence represented by SEQ ID NO: 1549.

The probes having the nucleotide sequence represented by SEQ ID NOS: 23020, 23021, 23022, 23023, 23024, 23025, 23026, 23027, 23028, 23029, 23030 were selected from the ORFs having the nucleotide sequence represented by SEQ ID NO: 1550.

The probes having the nucleotide sequence represented by SEQ ID NOS: 23031, 23032, 23033, 23034, 23035, 23036, 23037, 23038, 23039, 23040, 23041 were selected from the ORFs having the nucleotide sequence represented by SEQ ID NO: 1551.

The probes having the nucleotide sequence represented by SEQ ID NOS: 23042, 23043, 23044, 23045, 23046, 23047, 23048, 23049, 23050, 23051, 23052 were selected from the ORFs having the nucleotide sequence represented by SEQ ID NO: 1552.

The probes having the nucleotide sequence represented by SEQ ID NOS: 23053, 23054, 23055, 23056, 23057, 23058, 23059, 23060, 23061, 23062, 23063 were selected from the ORFs having the nucleotide sequence represented by SEQ ID NO: 1553.

The probes having the nucleotide sequence represented by SEQ ID NOS: 23064, 23065, 23066, 23067, 23068, 23069, 23070, 23071, 23072, 23073, 23074 were selected from the ORFs having the nucleotide sequence represented by SEQ ID NO: 1554.

The probes having the nucleotide sequence represented by SEQ ID NOS: 23075, 23076, 23077, 23078, 23079, 23080, 23081, 23082, 23083, 23084, 23085 were selected from the ORFs having the nucleotide sequence represented by SEQ ID NO: 1555.

The probes having the nucleotide sequence represented by SEQ ID NOS: 23086, 23087, 23088, 23089, 23090, 23091, 23092, 23093, 23094, 23095, 23096 were selected from the ORFs having the nucleotide sequence represented by SEQ ID NO: 1556.

The probes having the nucleotide sequence represented by SEQ ID NOS: 23097, 23098, 23099, 23100, 23101, 23102, 23103, 23104, 23105, 23106, 23107 were selected from the ORFs having the nucleotide sequence represented by SEQ ID NO: 1557.

The probes having the nucleotide sequence represented by SEQ ID NOS: 23108, 23109, 23110, 23111, 23112, 23113, 23114, 23115, 23116, 23117, 23118, 23119, 23120, 23121, 23122, 23123, 23124, 23125, 23126, 23127, 23128, 23129 were selected from the ORFs having the nucleotide sequence represented by SEQ ID NO: 1558.

The probes having the nucleotide sequence represented by SEQ ID NOS: 23130, 23131, 23132, 23133, 23134, 23135, 23136, 23137, 23138, 23139, 23140 were selected from the ORFs having the nucleotide sequence represented by SEQ ID NO: 1559.

The probes having the nucleotide sequence represented by SEQ ID NOS: 23141, 23142, 23143, 23144, 23145, 23146, 23147, 23148, 23149, 23150, 23151 were selected from the ORFs having the nucleotide sequence represented by SEQ ID NO: 1560.

The probes having the nucleotide sequence represented by SEQ ID NOS: 23152, 23153, 23154, 23155, 23156, 23157, 23158, 23159, 23160, 23161, 23162 were selected from the ORFs having the nucleotide sequence represented by SEQ ID NO: 1561.

The probes having the nucleotide sequence represented by SEQ ID NOS: 23163, 23164, 23165, 23166, 23167, 23168, 23169, 23170, 23171, 23172, 23173 were selected from the ORFs having the nucleotide sequence represented by SEQ ID NO: 1562.

The probes having the nucleotide sequence represented by SEQ ID NOS: 23174, 23175, 23176, 23177, 23178, 23179, 23180, 23181, 23182, 23183, 23184 were selected from the ORFs having the nucleotide sequence represented by SEQ ID NO: 1563.

The probes having the nucleotide sequence represented by SEQ ID NOS: 23185, 23186, 23187, 23188, 23189, 23190, 23191, 23192, 23193, 23194, 23195 were selected from the ORFs having the nucleotide sequence represented by SEQ ID NO: 1564.

The probes having the nucleotide sequence represented by SEQ ID NOS: 23196, 23197, 23198, 23199, 23200, 23201, 23202, 23203, 23204, 23205, 23206 were selected from the ORFs having the nucleotide sequence represented by SEQ ID NO: 1565.

The probes having the nucleotide sequence represented by SEQ ID NOS: 23207, 23208, 23209, 23210, 23211, 23212, 23213, 23214, 23215, 23216, 23217 were selected from the ORFs having the nucleotide sequence represented by SEQ ID NO: 1566.

The probes having the nucleotide sequence represented by SEQ ID NOS: 23218, 23219, 23220, 23221, 23222, 23223, 23224, 23225, 23226, 23227, 23228 were selected from the ORFs having the nucleotide sequence represented by SEQ ID NO: 1567.

The probes having the nucleotide sequence represented by SEQ ID NOS: 23229, 23230, 23231, 23232, 23233, 23234, 23235, 23236, 23237, 23238, 23239 were selected from the ORFs having the nucleotide sequence represented by SEQ ID NO: 1568.

The probes having the nucleotide sequence represented by SEQ ID NOS: 23240, 23241, 23242, 23243, 23244, 23245, 23246, 23247, 23248, 23249, 23250 were selected from the ORFs having the nucleotide sequence represented by SEQ ID NO: 1569.

The probes having the nucleotide sequence represented by SEQ ID NOS: 23251, 23252, 23253, 23254, 23255, 23256, 23257, 23258, 23259, 23260, 23261 were selected from the ORFs having the nucleotide sequence represented by SEQ ID NO: 1570.

The probes having the nucleotide sequence represented by SEQ ID NOS: 23262, 23263, 23264, 23265, 23266, 23267, 23268, 23269, 23270, 23271, 23272 were selected from the ORFs having the nucleotide sequence represented by SEQ ID NO: 1571.

The probes having the nucleotide sequence represented by SEQ ID NOS: 23273, 23274, 23275, 23276, 23277, 23278, 23279, 23280, 23281, 23282, 23283 were selected from the ORFs having the nucleotide sequence represented by SEQ ID NO: 1572.

The probes having the nucleotide sequence represented by SEQ ID NOS: 23284, 23285, 23286, 23287, 23288, 23289, 23290, 23291, 23292, 23293, 23294 were selected from the ORFs having the nucleotide sequence represented by SEQ ID NO: 1573.

The probes having the nucleotide sequence represented by SEQ ID NOS: 23295, 23296, 23297, 23298, 23299, 23300, 23301, 23302, 23303, 23304, 23305 were selected from the ORFs having the nucleotide sequence represented by SEQ ID NO: 1574.

The probes having the nucleotide sequence represented by SEQ ID NOS: 23306, 23307, 23308, 23309, 23310, 23311, 23312, 23313, 23314, 23315, 23316 were selected from the ORFs having the nucleotide sequence represented by SEQ ID NO: 1575.

The probes having the nucleotide sequence represented by SEQ ID NOS: 23317, 23318, 23319, 23320, 23321, 23322, 23323, 23324, 23325, 23326, 23327 were selected from the ORFs having the nucleotide sequence represented by SEQ ID NO: 1576.

The probes having the nucleotide sequence represented by SEQ ID NOS: 23328, 23329, 23330, 23331, 23332, 23333, 23334, 23335, 23336, 23337, 23338 were selected from the ORFs having the nucleotide sequence represented by SEQ ID NO: 1577.

The probes having the nucleotide sequence represented by SEQ ID NOS: 23339, 23340, 23341, 23342, 23343, 23344, 23345, 23346, 23347, 23348, 23349 were selected from the ORFs having the nucleotide sequence represented by SEQ ID NO: 1578.

The probes having the nucleotide sequence represented by SEQ ID NOS: 23350, 23351, 23352, 23353, 23354, 23355, 23356, 23357, 23358, 23359, 23360 were selected from the ORFs having the nucleotide sequence represented by SEQ ID NO: 1579.

The probes having the nucleotide sequence represented by SEQ ID NOS: 23361, 23362, 23363, 23364, 23365, 23366, 23367, 23368, 23369, 23370, 23371 were selected from the ORFs having the nucleotide sequence represented by SEQ ID NO: 1580.

The probes having the nucleotide sequence represented by SEQ ID NOS: 23372, 23373, 23374, 23375, 23376, 23377, 23378, 23379, 23380, 23381, 23382 were selected from the ORFs having the nucleotide sequence represented by SEQ ID NO: 1581.

The probes having the nucleotide sequence represented by SEQ ID NOS: 23383, 23384, 23385, 23386, 23387, 23388, 23389, 23390, 23391, 23392, 23393 were selected from the ORFs having the nucleotide sequence represented by SEQ ID NO: 1582.

The probes having the nucleotide sequence represented by SEQ ID NOS: 23394, 23395, 23396, 23397, 23398, 23399, 23400, 23401, 23402, 23403, 23404 were selected from the ORFs having the nucleotide sequence represented by SEQ ID NO: 1583.

The probes having the nucleotide sequence represented by SEQ ID NOS: 23405, 23406, 23407, 23408, 23409, 23410, 23411, 23412, 23413, 23414, 23415 were selected from the ORFs having the nucleotide sequence represented by SEQ ID NO: 1584.

The probes having the nucleotide sequence represented by SEQ ID NOS: 23416, 23417, 23418, 23419, 23420, 23421, 23422, 23423, 23424, 23425, 23426 were selected from the ORFs having the nucleotide sequence represented by SEQ ID NO: 1585.

The probes having the nucleotide sequence represented by SEQ ID NOS: 23427, 23428, 23429, 23430, 23431, 23432, 23433, 23434, 23435, 23436, 23437 were selected from the ORFs having the nucleotide sequence represented by SEQ ID NO: 1586.

The probes having the nucleotide sequence represented by SEQ ID NOS: 23438, 23439, 23440, 23441, 23442, 23443, 23444, 23445, 23446, 23447, 23448 were selected from the ORFs having the nucleotide sequence represented by SEQ ID NO: 1587.

The probes having the nucleotide sequence represented by SEQ ID NOS: 23449, 23450, 23451, 23452, 23453, 23454, 23455, 23456, 23457, 23458, 23459 were selected from the ORFs having the nucleotide sequence represented by SEQ ID NO: 1588.

The probes having the nucleotide sequence represented by SEQ ID NOS: 23460, 23461, 23462, 23463, 23464, 23465, 23466, 23467, 23468, 23469, 23470 were selected from the ORFs having the nucleotide sequence represented by SEQ ID NO: 1589.

The probes having the nucleotide sequence represented by SEQ ID NOS: 23471, 23472, 23473, 23474, 23475, 23476, 23477, 23478, 23479, 23480, 23481 were selected from the ORFs having the nucleotide sequence represented by SEQ ID NO: 1590.

The probes having the nucleotide sequence represented by SEQ ID NOS: 23482, 23483, 23484, 23485, 23486, 23487, 23488, 23489, 23490, 23491, 23492 were selected from the ORFs having the nucleotide sequence represented by SEQ ID NO: 1591.

The probes having the nucleotide sequence represented by SEQ ID NOS: 23493, 23494, 23495, 23496, 23497, 23498, 23499, 23500, 23501, 23502, 23503 were selected from the ORFs having the nucleotide sequence represented by SEQ ID NO: 1592.

The probes having the nucleotide sequence represented by SEQ ID NOS: 23504, 23505, 23506, 23507, 23508, 23509, 23510, 23511, 23512, 23513, 23514 were selected from the ORFs having the nucleotide sequence represented by SEQ ID NO: 1593.

The probes having the nucleotide sequence represented by SEQ ID NOS: 23515, 23516, 23517, 23518, 23519, 23520, 23521, 23522, 23523, 23524, 23525 were selected from the ORFs having the nucleotide sequence represented by SEQ ID NO: 1594.

The probes having the nucleotide sequence represented by SEQ ID NOS: 23526, 23527, 23528, 23529, 23530, 23531, 23532, 23533, 23534, 23535, 23536 were selected from the ORFs having the nucleotide sequence represented by SEQ ID NO: 1595.

The probes having the nucleotide sequence represented by SEQ ID NOS: 23537, 23538, 23539, 23540, 23541, 23542, 23543, 23544, 23545, 23546, 23547 were selected from the ORFs having the nucleotide sequence represented by SEQ ID NO: 1596.

The probes having the nucleotide sequence represented by SEQ ID NOS: 23548, 23549, 23550, 23551, 23552, 23553, 23554, 23555, 23556, 23557, 23558 were selected from the ORFs having the nucleotide sequence represented by SEQ ID NO: 1597.

The probes having the nucleotide sequence represented by SEQ ID NOS: 23559, 23560, 23561, 23562, 23563, 23564, 23565, 23566, 23567, 23568, 23569 were selected from the ORFs having the nucleotide sequence represented by SEQ ID NO: 1598.

The probes having the nucleotide sequence represented by SEQ ID NOS: 23570, 23571, 23572, 23573, 23574, 23575, 23576, 23577, 23578, 23579, 23580 were selected from the ORFs having the nucleotide sequence represented by SEQ ID NO: 1599.

The probes having the nucleotide sequence represented by SEQ ID NOS: 23581, 23582, 23583, 23584, 23585, 23586, 23587, 23588, 23589, 23590, 23591 were selected from the ORFs having the nucleotide sequence represented by SEQ ID NO: 1600.

The probes having the nucleotide sequence represented by SEQ ID NOS: 23592, 23593, 23594, 23595, 23596, 23597, 23598, 23599, 23600, 23601, 23602 were selected from the ORFs having the nucleotide sequence represented by SEQ ID NO: 1601.

The probes having the nucleotide sequence represented by SEQ ID NOS: 23603, 23604, 23605, 23606, 23607, 23608, 23609, 23610, 23611, 23612, 23613 were selected from the ORFs having the nucleotide sequence represented by SEQ ID NO: 1602.

The probes having the nucleotide sequence represented by SEQ ID NOS: 23614, 23615, 23616, 23617, 23618, 23619, 23620, 23621, 23622, 23623, 23624 were selected from the ORFs having the nucleotide sequence represented by SEQ ID NO: 1603.

The probes having the nucleotide sequence represented by SEQ ID NOS: 23625, 23626, 23627, 23628, 23629, 23630, 23631, 23632, 23633, 23634, 23635 were selected from the ORFs having the nucleotide sequence represented by SEQ ID NO: 1604.

The probes having the nucleotide sequence represented by SEQ ID NOS: 23636, 23637, 23638, 23639, 23640, 23641, 23642, 23643, 23644, 23645, 23646 were selected from the ORFs having the nucleotide sequence represented by SEQ ID NO: 1605.

The probes having the nucleotide sequence represented by SEQ ID NOS: 23647, 23648, 23649, 23650, 23651, 23652, 23653, 23654, 23655, 23656, 23657 were selected from the ORFs having the nucleotide sequence represented by SEQ ID NO: 1606.

The probes having the nucleotide sequence represented by SEQ ID NOS: 23658, 23659, 23660, 23661, 23662, 23663, 23664, 23665, 23666, 23667, 23668 were selected from the ORFs having the nucleotide sequence represented by SEQ ID NO: 1607.

The probes having the nucleotide sequence represented by SEQ ID NOS: 23669, 23670, 23671, 23672, 23673, 23674, 23675, 23676, 23677, 23678, 23679 were selected from the ORFs having the nucleotide sequence represented by SEQ ID NO: 1608.

The probes having the nucleotide sequence represented by SEQ ID NOS: 23680, 23681, 23682, 23683, 23684, 23685, 23686, 23687, 23688, 23689, 23690 were selected from the ORFs having the nucleotide sequence represented by SEQ ID NO: 1609.

The probes having the nucleotide sequence represented by SEQ ID NOS: 23691, 23692, 23693, 23694, 23695, 23696, 23697, 23698, 23699, 23700, 23701 were selected from the ORFs having the nucleotide sequence represented by SEQ ID NO: 1610.

The probes having the nucleotide sequence represented by SEQ ID NOS: 23702, 23703, 23704, 23705, 23706, 23707, 23708, 23709, 23710, 23711, 23712 were selected from the ORFs having the nucleotide sequence represented by SEQ ID NO: 1611.

The probes having the nucleotide sequence represented by SEQ ID NOS: 23713, 23714, 23715, 23716, 23717, 23718, 23719, 23720, 23721, 23722, 23723 were selected from the ORFs having the nucleotide sequence represented by SEQ ID NO: 1612.

The probes having the nucleotide sequence represented by SEQ ID NOS: 23724, 23725, 23726, 23727, 23728, 23729, 23730, 23731, 23732, 23733, 23734 were selected from the ORFs having the nucleotide sequence represented by SEQ ID NO: 1613.

The probes having the nucleotide sequence represented by SEQ ID NOS: 23735, 23736, 23737, 23738, 23739, 23740, 23741, 23742, 23743, 23744, 23745 were selected from the ORFs having the nucleotide sequence represented by SEQ ID NO: 1614.

The probes having the nucleotide sequence represented by SEQ ID NOS: 23746, 23747, 23748, 23749, 23750, 23751, 23752, 23753, 23754, 23755, 23756 were selected from the ORFs having the nucleotide sequence represented by SEQ ID NO: 1615.

The probes having the nucleotide sequence represented by SEQ ID NOS: 23757, 23758, 23759, 23760, 23761, 23762, 23763, 23764, 23765, 23766, 23767 were selected from the ORFs having the nucleotide sequence represented by SEQ ID NO: 1616.

The probes having the nucleotide sequence represented by SEQ ID NOS: 23768, 23769, 23770, 23771, 23772, 23773, 23774, 23775, 23776, 23777, 23778 were selected from the ORFs having the nucleotide sequence represented by SEQ ID NO: 1617.

The probes having the nucleotide sequence represented by SEQ ID NOS: 23779, 23780, 23781, 23782, 23783, 23784, 23785, 23786, 23787, 23788, 23789 were selected from the ORFs having the nucleotide sequence represented by SEQ ID NO: 1618.

The probes having the nucleotide sequence represented by SEQ ID NOS: 23790, 23791, 23792, 23793, 23794, 23795, 23796, 23797, 23798, 23799, 23800 were selected from the ORFs having the nucleotide sequence represented by SEQ ID NO: 1619.

The probes having the nucleotide sequence represented by SEQ ID NOS: 23801, 23802, 23803, 23804, 23805, 23806, 23807, 23808, 23809, 23810, 23811 were selected from the ORFs having the nucleotide sequence represented by SEQ ID NO: 1620.

The probes having the nucleotide sequence represented by SEQ ID NOS: 23812, 23813, 23814, 23815, 23816, 23817, 23818, 23819, 23820, 23821, 23822 were selected from the ORFs having the nucleotide sequence represented by SEQ ID NO: 1621.

The probes having the nucleotide sequence represented by SEQ ID NOS: 23823, 23824, 23825, 23826, 23827, 23828, 23829, 23830, 23831, 23832, 23833 were selected from the ORFs having the nucleotide sequence represented by SEQ ID NO: 1622.

The probes having the nucleotide sequence represented by SEQ ID NOS: 23834, 23835, 23836, 23837, 23838, 23839, 23840, 23841, 23842, 23843, 23844 were selected from the ORFs having the nucleotide sequence represented by SEQ ID NO: 1623.

The probes having the nucleotide sequence represented by SEQ ID NOS: 23845, 23846, 23847, 23848, 23849, 23850, 23851, 23852, 23853, 23854, 23855 were selected from the ORFs having the nucleotide sequence represented by SEQ ID NO: 1624.

The probes having the nucleotide sequence represented by SEQ ID NOS: 23856, 23857, 23858, 23859, 23860, 23861, 23862, 23863, 23864, 23865, 23866 were selected from the ORFs having the nucleotide sequence represented by SEQ ID NO: 1625.

The probes having the nucleotide sequence represented by SEQ ID NOS: 23867, 23868, 23869, 23870, 23871, 23872, 23873, 23874, 23875, 23876, 23877 were selected from the ORFs having the nucleotide sequence represented by SEQ ID NO: 1626.

The probes having the nucleotide sequence represented by SEQ ID NOS: 23878, 23879, 23880, 23881, 23882, 23883, 23884, 23885, 23886, 23887, 23888 were selected from the ORFs having the nucleotide sequence represented by SEQ ID NO: 1627.

The probes having the nucleotide sequence represented by SEQ ID NOS: 23889, 23890, 23891, 23892, 23893, 23894, 23895, 23896, 23897, 23898, 23899 were selected from the ORFs having the nucleotide sequence represented by SEQ ID NO: 1628.

The probes having the nucleotide sequence represented by SEQ ID NOS: 23900, 23901, 23902, 23903, 23904, 23905, 23906, 23907, 23908, 23909, 23910 were selected from the ORFs having the nucleotide sequence represented by SEQ ID NO: 1629.

The probes having the nucleotide sequence represented by SEQ ID NOS: 23911, 23912, 23913, 23914, 23915, 23916, 23917, 23918, 23919, 23920, 23921 were selected from the ORFs having the nucleotide sequence represented by SEQ ID NO: 1630.

The probes having the nucleotide sequence represented by SEQ ID NOS: 23922; 23923, 23924, 23925, 23926, 23927, 23928, 23929, 23930, 23931, 23932 were selected from the ORFs having the nucleotide sequence represented by SEQ ID NO: 1631.

The probes having the nucleotide sequence represented by SEQ ID NOS: 23933, 23934, 23935, 23936, 23937, 23938, 23939, 23940, 23941, 23942, 23943 were selected from the ORFs having the nucleotide sequence represented by SEQ ID NO: 1632.

The probes having the nucleotide sequence represented by SEQ ID NOS: 23944, 23945, 23946, 23947, 23948, 23949, 23950, 23951, 23952, 23953, 23954 were selected from the ORFs having the nucleotide sequence represented by SEQ ID NO: 1633.

The probes having the nucleotide sequence represented by SEQ ID NOS: 23955, 23956, 23957, 23958, 23959, 23960, 23961, 23962, 23963, 23964, 23965 were selected from the ORFs having the nucleotide sequence represented by SEQ ID NO: 1634.

The probes having the nucleotide sequence represented by SEQ ID NOS: 23966, 23967, 23968, 23969, 23970, 23971, 23972, 23973, 23974, 23975, 23976 were selected from the ORFs having the nucleotide sequence represented by SEQ ID NO: 1635.

The probes having the nucleotide sequence represented by SEQ ID NOS: 23977, 23978, 23979, 23980, 23981, 23982, 23983, 23984, 23985, 23986, 23987 were selected from the ORFs having the nucleotide sequence represented by SEQ ID NO: 1636.

The probes having the nucleotide sequence represented by SEQ ID NOS: 23988, 23989, 23990, 23991, 23992, 23993, 23994, 23995, 23996, 23997, 23998 were selected from the ORFs having the nucleotide sequence represented by SEQ ID NO: 1637.

The probes having the nucleotide sequence represented by SEQ ID NOS: 23999, 24000, 24001, 24002, 24003, 24004, 24005, 24006, 24007, 24008, 24009 were selected from the ORFs having the nucleotide sequence represented by SEQ ID NO: 1638.

The probes having the nucleotide sequence represented by SEQ ID NOS: 24010, 24011, 24012, 24013, 24014, 24015, 24016, 24017, 24018, 24019, 24020 were selected from the ORFs having the nucleotide sequence represented by SEQ ID NO: 1639.

The probes having the nucleotide sequence represented by SEQ ID NOS: 24021, 24022, 24023, 24024, 24025, 24026, 24027, 24028, 24029, 24030, 24031 were selected from the ORFs having the nucleotide sequence represented by SEQ ID NO: 1640.

The probes having the nucleotide sequence represented by SEQ ID NOS: 24032, 24033, 24034, 24035, 24036, 24037, 24038, 24039, 24040, 24041, 24042 were selected from the ORFs having the nucleotide sequence represented by SEQ ID NO: 1641.

The probes having the nucleotide sequence represented by SEQ ID NOS: 24043, 24044, 24045, 24046, 24047, 24048, 24049, 24050, 24051, 24052, 24053 were selected from the ORFs having the nucleotide sequence represented by SEQ ID NO: 1642.

The probes having the nucleotide sequence represented by SEQ ID NOS: 24054, 24055, 24056, 24057, 24058, 24059, 24060, 24061, 24062, 24063, 24064 were selected from the ORFs having the nucleotide sequence represented by SEQ ID NO: 1643.

The probes having the nucleotide sequence represented by SEQ ID NOS: 24065, 24066, 24067, 24068, 24069, 24070, 24071, 24072, 24073, 24074, 24075 were selected from the ORFs having the nucleotide sequence represented by SEQ ID NO: 1644.

The probes having the nucleotide sequence represented by SEQ ID NOS: 24076, 24077, 24078, 24079, 24080, 24081, 24082, 24083, 24084, 24085, 24086 were selected from the ORFs having the nucleotide sequence represented by SEQ ID NO: 1645.

The probes having the nucleotide sequence represented by SEQ ID NOS: 24087, 24088, 24089, 24090, 24091, 24092, 24093, 24094, 24095, 24096, 24097 were selected from the ORFs having the nucleotide sequence represented by SEQ ID NO: 1646.

The probes having the nucleotide sequence represented by SEQ ID NOS: 24098, 24099, 24100, 24101, 24102, 24103, 24104, 24105, 24106, 24107, 24108 were selected from the ORFs having the nucleotide sequence represented by SEQ ID NO: 1647.

The probes having the nucleotide sequence represented by SEQ ID NOS: 24109, 24110, 24111, 24112, 24113, 24114, 24115, 24116, 24117, 24118, 24119 were selected from the ORFs having the nucleotide sequence represented by SEQ ID NO: 1648.

The probes having the nucleotide sequence represented by SEQ ID NOS: 24120, 24121, 24122, 24123, 24124, 24125, 24126, 24127, 24128, 24129, 24130 were selected from the ORFs having the nucleotide sequence represented by SEQ ID NO: 1649.

The probes having the nucleotide sequence represented by SEQ ID NOS: 24131, 24132, 24133, 24134, 24135, 24136, 24137, 24138, 24139, 24140, 24141 were selected from the ORFs having the nucleotide sequence represented by SEQ ID NO: 1650.

The probes having the nucleotide sequence represented by SEQ ID NOS: 24142, 24143, 24144, 24145, 24146, 24147, 24148, 24149, 24150, 24151, 24152 were selected from the ORFs having the nucleotide sequence represented by SEQ ID NO: 1651.

The probes having the nucleotide sequence represented by SEQ ID NOS: 24153, 24154, 24155, 24156, 24157, 24158, 24159, 24160, 24161, 24162, 24163 were selected from the ORFs having the nucleotide sequence represented by SEQ ID NO: 1652.

The probes having the nucleotide sequence represented by SEQ ID NOS: 24164, 24165, 24166, 24167, 24168, 24169, 24170, 24171, 24172, 24173, 24174 were selected from the ORFs having the nucleotide sequence represented by SEQ ID NO: 1653.

The probes having the nucleotide sequence represented by SEQ ID NOS: 24175, 24176, 24177, 24178, 24179, 24180, 24181, 24182, 24183, 24184, 24185 were selected from the ORFs having the nucleotide sequence represented by SEQ ID NO: 1654.

The probes having the nucleotide sequence represented by SEQ ID NOS: 24186, 24187, 24188, 24189, 24190, 24191, 24192, 24193, 24194, 24195, 24196 were selected from the ORFs having the nucleotide sequence represented by SEQ ID NO: 1655.

The probes having the nucleotide sequence represented by SEQ ID. NOS: 24197, 24198, 24199, 24200, 24201, 24202, 24203, 24204, 24205, 24206, 24207 were selected from the ORFs having the nucleotide sequence represented by SEQ ID NO: 1656.

The probes having the nucleotide sequence represented by SEQ ID NOS: 24208, 24209, 24210, 24211, 24212, 24213, 24214, 24215, 24216, 24217, 24218 were selected from the ORFs having the nucleotide sequence represented by SEQ ID NO: 1657.

The probes having the nucleotide sequence represented by SEQ ID NOS: 24219, 24220, 24221, 24222, 24223, 24224, 24225, 24226, 24227, 24228, 24229 were selected from the ORFs having the nucleotide sequence represented by SEQ ID NO: 1658.

The probes having the nucleotide sequence represented by SEQ ID NOS: 24230, 24231, 24232, 24233, 24234, 24235, 24236, 24237, 24238, 24239, 24240 were selected from the ORFs having the nucleotide sequence represented by SEQ ID NO: 1659.

The probes having the nucleotide sequence represented by SEQ ID NOS: 24241, 24242, 24243, 24244, 24245, 24246, 24247, 24248, 24249, 24250, 24251 were selected from the ORFs having the nucleotide sequence represented by SEQ ID NO: 1660.

The probes having the nucleotide sequence represented by SEQ ID NOS: 24252, 24253, 24254, 24255, 24256, 24257, 24258, 24259, 24260, 24261, 24262 were selected from the ORFs having the nucleotide sequence represented by SEQ ID NO: 1661.

The probes having the nucleotide sequence represented by SEQ ID NOS: 24263, 24264, 24265, 24266, 24267, 24268, 24269, 24270, 24271, 24272, 24273 were selected from the ORFs having the nucleotide sequence represented by SEQ ID NO: 1662.

The probes having the nucleotide sequence represented by SEQ ID NOS: 24274, 24275, 24276, 24277, 24278, 24279, 24280, 24281, 24282, 24283, 24284, 24285, 24286, 24287, 24288, 24289, 24290, 24291, 24292, 24293, 24294, 24295 were selected from the ORFs having the nucleotide sequence represented by SEQ ID NO: 1663.

The probes having the nucleotide sequence represented by SEQ ID NOS: 24296, 24297, 24298, 24299, 24300, 24301, 24302, 24303, 24304, 24305, 24306 were selected from the ORFs having the nucleotide sequence represented by SEQ ID NO: 1664.

The probes having the nucleotide sequence represented by SEQ ID NOS: 24307, 24308, 24309, 24310, 24311, 24312, 24313, 24314, 24315, 24316, 24317 were selected from the ORFs having the nucleotide sequence represented by SEQ ID NO: 1665.

The probes having the nucleotide sequence represented by SEQ ID NOS: 24318, 24319, 24320, 24321, 24322, 24323, 24324, 24325, 24326, 24327, 24328 were selected from the ORFs having the nucleotide sequence represented by SEQ ID NO: 1666.

The probes having the nucleotide sequence represented by SEQ ID NOS: 24329, 24330, 24331, 24332, 24333, 24334, 24335, 24336, 24337, 24338, 24339 were selected from the ORFs having the nucleotide sequence represented by SEQ ID NO: 1667.

The probes having the nucleotide sequence represented by SEQ ID NOS: 24340, 24341, 24342, 24343, 24344, 24345, 24346, 24347, 24348, 24349, 24350 were selected from the ORFs having the nucleotide sequence represented by SEQ ID NO: 1668.

The probes having the nucleotide sequence represented by SEQ ID NOS: 24351, 24352, 24353, 24354, 24355, 24356, 24357, 24358, 24359, 24360, 24361 were selected from the ORFs having the nucleotide sequence represented by SEQ ID NO: 1669.

The probes having the nucleotide sequence represented by SEQ ID NOS: 24362, 24363, 24364, 24365, 24366, 24367, 24368, 24369, 24370, 24371, 24372 were selected from the ORFs having the nucleotide sequence represented by SEQ ID NO: 1670.

The probes having the nucleotide sequence represented by SEQ ID NOS: 24373, 24374, 24375, 24376, 24377, 24378, 24379, 24380, 24381, 24382, 24383 were selected from the ORFs having the nucleotide sequence represented by SEQ ID NO: 1671.

The probes having the nucleotide sequence represented by SEQ ID NOS: 24384, 24385, 24386, 24387, 24388, 24389, 24390, 24391, 24392, 24393, 24394 were selected from the ORFs having the nucleotide sequence represented by SEQ ID NO: 1672.

The probes having the nucleotide sequence represented by SEQ ID NOS: 24395, 24396, 24397, 24398, 24399, 24400, 24401, 24402, 24403, 24404, 24405 were selected from the ORFs having the nucleotide sequence represented by SEQ ID NO: 1673.

The probes having the nucleotide sequence represented by SEQ ID NOS: 24406, 24407, 24408, 24409, 24410, 24411, 24412, 24413, 24414, 24415, 24416 were selected from the ORFs having the nucleotide sequence represented by SEQ ID NO: 1674.

The probes having the nucleotide sequence represented by SEQ ID NOS: 24417, 24418, 24419, 24420, 24421, 24422, 24423, 24424, 24425, 24426, 24427 were selected from the ORFs having the nucleotide sequence represented by SEQ ID NO: 1675.

The probes having the nucleotide sequence represented by SEQ ID NOS: 24428, 24429, 24430, 24431, 24432, 24433, 24434, 24435, 24436, 24437, 24438 were selected from the ORFs having the nucleotide sequence represented by SEQ ID NO: 1676.

The probes having the nucleotide sequence represented by SEQ ID NOS: 24439, 24440, 24441, 24442, 24443, 24444, 24445, 24446, 24447, 24448, 24449 were selected from the ORFs having the nucleotide sequence represented by SEQ ID NO: 1677.

The probes having the nucleotide sequence represented by SEQ ID NOS: 24450, 24451, 24452, 24453, 24454, 24455, 24456, 24457, 24458, 24459, 24460 were selected from the ORFs having the nucleotide sequence represented by SEQ ID NO: 1678.

The probes having the nucleotide sequence represented by SEQ ID NOS: 24461, 24462, 24463, 24464, 24465, 24466, 24467, 24468, 24469, 24470, 24471 were selected from the ORFs having the nucleotide sequence represented by SEQ ID NO: 1679.

The probes having the nucleotide sequence represented by SEQ ID NOS: 24472, 24473, 24474, 24475, 24476, 24477, 24478, 24479, 24480, 24481, 24482 were selected from the ORFs having the nucleotide sequence represented by SEQ ID NO: 1680.

The probes having the nucleotide sequence represented by SEQ ID NOS: 24483, 24484, 24485, 24486, 24487, 24488, 24489, 24490, 24491, 24492, 24493 were selected from the ORFs having the nucleotide sequence represented by SEQ ID NO: 1681.

The probes having the nucleotide sequence represented by SEQ ID NOS: 24494, 24495, 24496, 24497, 24498, 24499, 24500, 24501, 24502, 24503, 24504 were selected from the ORFs having the nucleotide sequence represented by SEQ ID NO: 1682.

The probes having the nucleotide sequence represented by SEQ ID NOS: 24505, 24506, 24507, 24508, 24509, 24510, 24511, 24512, 24513, 24514, 24515 were selected from the ORFs having the nucleotide sequence represented by SEQ ID NO: 1683.

The probes having the nucleotide sequence represented by SEQ ID NOS: 24516, 24517, 24518, 24519, 24520, 24521, 24522, 24523, 24524, 24525, 24526 were selected from the ORFs having the nucleotide sequence represented by SEQ ID NO: 1684.

The probes having the nucleotide sequence represented by SEQ ID NOS: 24527, 24528, 24529, 24530, 24531, 24532, 24533, 24534, 24535, 24536, 24537 were selected from the ORFs having the nucleotide sequence represented by SEQ ID NO: 1685.

The probes having the nucleotide sequence represented by SEQ ID NOS: 24538, 24539, 24540, 24541, 24542, 24543, 24544, 24545, 24546, 24547, 24548 were selected from the ORFs having the nucleotide sequence represented by SEQ ID NO: 1686.

The probes having the nucleotide sequence represented by SEQ ID NOS: 24549, 24550, 24551, 24552, 24553, 24554, 24555, 24556, 24557, 24558, 24559 were selected from the ORFs having the nucleotide sequence represented by SEQ ID NO: 1687.

The probes having the nucleotide sequence represented by SEQ ID NOS: 24560, 24561, 24562, 24563, 24564, 24565, 24566, 24567, 24568, 24569, 24570 were selected from the ORFs having the nucleotide sequence represented by SEQ ID NO: 1688.

The probes having the nucleotide sequence represented by SEQ ID NOS: 24571, 24572, 24573, 24574, 24575, 24576, 24577, 24578, 24579, 24580, 24581 were selected from the ORFs having the nucleotide sequence represented by SEQ ID NO: 1689.

The probes having the nucleotide sequence represented by SEQ ID NOS: 24582, 24583, 24584, 24585, 24586, 24587, 24588, 24589, 24590, 24591, 24592 were selected from the ORFs having the nucleotide sequence represented by SEQ ID NO: 1690.

The probes having the nucleotide sequence represented by SEQ ID NOS: 24593, 24594, 24595, 24596, 24597, 24598, 24599, 24600, 24601, 24602, 24603 were selected from the ORFs having the nucleotide sequence represented by SEQ ID NO: 1691.

The probes having the nucleotide sequence represented by SEQ ID NOS: 24604, 24605, 24606, 24607, 24608, 24609, 24610, 24611, 24612, 24613, 24614 were selected from the ORFs having the nucleotide sequence represented by SEQ ID NO: 1692.

The probes having the nucleotide sequence represented by SEQ ID NOS: 24615, 24616, 24617, 24618, 24619, 24620, 24621, 24622, 24623, 24624, 24625 were selected from the ORFs having the nucleotide sequence represented by SEQ ID NO: 1693.

The probes having the nucleotide sequence represented by SEQ ID NOS: 24626, 24627, 24628, 24629, 24630, 24631, 24632, 24633, 24634, 24635, 24636 were selected from the ORFs having the nucleotide sequence represented by SEQ ID NO: 1694.

The probes having the nucleotide sequence represented by SEQ ID NOS: 24637, 24638, 24639, 24640, 24641, 24642, 24643, 24644, 24645, 24646, 24647 were selected from the ORFs having the nucleotide sequence represented by SEQ ID NO: 1695.

The probes having the nucleotide sequence represented by SEQ ID NOS: 24648, 24649, 24650, 24651, 24652, 24653, 24654, 24655, 24656, 24657, 24658 were selected from the ORFs having the nucleotide sequence represented by SEQ ID NO: 1696.

The probes having the nucleotide sequence represented by SEQ ID NOS: 24659, 24660, 24661, 24662, 24663, 24664, 24665, 24666, 24667, 24668, 24669 were selected from the ORFs having the nucleotide sequence represented by SEQ ID NO: 1697.

The probes having the nucleotide sequence represented by SEQ ID NOS: 24670, 24671, 24672, 24673, 24674, 24675, 24676, 24677, 24678, 24679, 24680 were selected from the ORFs having the nucleotide sequence represented by SEQ ID NO: 1698.

The probes having the nucleotide sequence represented by SEQ ID NOS: 24681, 24682, 24683, 24684, 24685, 24686, 24687, 24688, 24689, 24690, 24691 were selected from the ORFs having the nucleotide sequence represented by SEQ ID NO: 1699.

The probes having the nucleotide sequence represented by SEQ ID NOS: 24692, 24693, 24694, 24695, 24696, 24697, 24698, 24699, 24700, 24701, 24702 were selected from the ORFs having the nucleotide sequence represented by SEQ ID NO: 1700.

The probes having the nucleotide sequence represented by SEQ ID NOS: 24703, 24704, 24705, 24706, 24707, 24708, 24709, 24710, 24711, 24712, 24713 were selected from the ORFs having the nucleotide sequence represented by SEQ ID NO: 1701.

The probes having the nucleotide sequence represented by SEQ ID NOS: 24714, 24715, 24716, 24717, 24718, 24719, 24720, 24721, 24722, 24723, 24724 were selected from the ORFs having the nucleotide sequence represented by SEQ ID NO: 1702.

The probes having the nucleotide sequence represented by SEQ ID NOS: 24725, 24726, 24727, 24728, 24729, 24730, 24731, 24732, 24733, 24734, 24735 were selected from the ORFs having the nucleotide sequence represented by SEQ ID NO: 1703.

The probes having the nucleotide sequence represented by SEQ ID NOS: 24736, 24737, 24738, 24739, 24740, 24741, 24742, 24743, 24744, 24745, 24746 were selected from the ORFs having the nucleotide sequence represented by SEQ ID NO: 1704.

The probes having the nucleotide sequence represented by SEQ ID NOS: 24747, 24748, 24749, 24750, 24751, 24752, 24753, 24754, 24755, 24756, 24757 were selected from the ORFs having the nucleotide sequence represented by SEQ ID NO: 1705.

The probes having the nucleotide sequence represented by SEQ ID NOS: 24758, 24759, 24760, 24761, 24762, 24763, 24764, 24765, 24766, 24767, 24768 were selected from the ORFs having the nucleotide sequence represented by SEQ ID NO: 1706.

The probes having the nucleotide sequence represented by SEQ ID NOS: 24769, 24770, 24771, 24772, 24773, 24774, 24775, 24776, 24777, 24778, 24779 were selected from the ORFs having the nucleotide sequence represented by SEQ ID NO: 1707.

The probes having the nucleotide sequence represented by SEQ ID NOS: 24780, 24781, 24782, 24783, 24784, 24785, 24786, 24787, 24788, 24789, 24790 were selected from the ORFs having the nucleotide sequence represented by SEQ ID NO: 1708.

The probes having the nucleotide sequence represented by SEQ ID NOS: 24791, 24792, 24793, 24794, 24795, 24796, 24797, 24798, 24799, 24800, 24801 were selected from the ORFs having the nucleotide sequence represented by SEQ ID NO: 1709.

The probes having the nucleotide sequence represented by SEQ ID NOS: 24802, 24803, 24804, 24805, 24806, 24807, 24808, 24809, 24810, 24811, 24812 were selected from the ORFs having the nucleotide sequence represented by SEQ ID NO: 1710.

The probes having the nucleotide sequence represented by SEQ ID NOS: 24813, 24814, 24815, 24816, 24817, 24818, 24819, 24820, 24821, 24822, 24823 were selected from the ORFs having the nucleotide sequence represented by SEQ ID NO: 1711.

The probes having the nucleotide sequence represented by SEQ ID NOS: 24824, 24825, 24826, 24827, 24828, 24829, 24830, 24831, 24832, 24833, 24834 were selected from the ORFs having the nucleotide sequence represented by SEQ ID NO: 1712.

The probes having the nucleotide sequence represented by SEQ ID NOS: 24835, 24836, 24837, 24838, 24839, 24840, 24841, 24842, 24843, 24844, 24845 were selected from the ORFs having the nucleotide sequence represented by SEQ ID NO: 1713.

The probes having the nucleotide sequence represented by SEQ ID NOS: 24846, 24847, 24848, 24849, 24850, 24851, 24852, 24853, 24854, 24855, 24856 were selected from the ORFs having the nucleotide sequence represented by SEQ ID NO: 1714.

The probes having the nucleotide sequence represented by SEQ ID NOS: 24857, 24858, 24859, 24860, 24861, 24862, 24863, 24864, 24865, 24866, 24867 were selected from the ORFs having the nucleotide sequence represented by SEQ ID NO: 1715.

The probes having the nucleotide sequence represented by SEQ ID NOS: 24868, 24869, 24870, 24871, 24872, 24873, 24874, 24875, 24876, 24877, 24878 were selected from the ORFs having the nucleotide sequence represented by SEQ ID NO: 1716.

The probes having the nucleotide sequence represented by SEQ ID NOS: 24879, 24880, 24881, 24882, 24883, 24884, 24885, 24886, 24887, 24888, 24889 were selected from the ORFs having the nucleotide sequence represented by SEQ ID NO: 1717.

The probes having the nucleotide sequence represented by SEQ ID NOS: 24890, 24891, 24892, 24893, 24894, 24895, 24896, 24897, 24898, 24899, 24900 were selected from the ORFs having the nucleotide sequence represented by SEQ ID NO: 1718.

The probes having the nucleotide sequence represented by SEQ ID NOS: 24901, 24902, 24903, 24904, 24905, 24906, 24907, 24908, 24909, 24910, 24911 were selected from the ORFs having the nucleotide sequence represented by SEQ ID NO: 1719.

The probes having the nucleotide sequence represented by SEQ ID NOS: 24912, 24913, 24914, 24915, 24916, 24917, 24918, 24919, 24920, 24921, 24922 were selected from the ORFs having the nucleotide sequence represented by SEQ ID NO: 1720.

The probes having the nucleotide sequence represented by SEQ ID NOS: 24923, 24924, 24925, 24926, 24927, 24928, 24929, 24930, 24931, 24932, 24933 were selected from the ORFs having the nucleotide sequence represented by SEQ ID NO: 1721.

The probes having the nucleotide sequence represented by SEQ ID NOS: 24934, 24935, 24936, 24937, 24938, 24939, 24940, 24941, 24942, 24943, 24944 were selected from the ORFs having the nucleotide sequence represented by SEQ ID NO: 1722.

The probes having the nucleotide sequence represented by SEQ ID NOS: 24945, 24946, 24947, 24948, 24949, 24950, 24951, 24952, 24953, 24954, 24955 were selected from the ORFs having the nucleotide sequence represented by SEQ ID NO: 1723.

The probes having the nucleotide sequence represented by SEQ ID NOS: 24956, 24957, 24958, 24959, 24960, 24961, 24962, 24963, 24964, 24965, 24966 were selected from the ORFs having the nucleotide sequence represented by SEQ ID NO: 1724.

The probes having the nucleotide sequence represented by SEQ ID NOS: 24967, 24968, 24969, 24970, 24971, 24972, 24973, 24974, 24975, 24976, 24977 were selected from the ORFs having the nucleotide sequence represented by SEQ ID NO: 1725.

The probes having the nucleotide sequence represented by SEQ ID NOS: 24978, 24979, 24980, 24981, 24982, 24983, 24984, 24985, 24986, 24987, 24988 were selected from the ORFs having the nucleotide sequence represented by SEQ ID NO: 1726.

The probes having the nucleotide sequence represented by SEQ ID NOS: 24989, 24990, 24991, 24992, 24993, 24994, 24995, 24996, 24997, 24998, 24999 were selected from the ORFs having the nucleotide sequence represented by SEQ ID NO: 1727.

The probes having the nucleotide sequence represented by SEQ ID NOS: 25000, 25001, 25002, 25003, 25004, 25005, 25006, 25007, 25008, 25009, 25010 were selected from the ORFs having the nucleotide sequence represented by SEQ ID NO: 1728.

The probes having the nucleotide sequence represented by SEQ ID NOS: 25011, 25012, 25013, 25014, 25015, 25016, 25017, 25018, 25019, 25020, 25021 were selected from the ORFs having the nucleotide sequence represented by SEQ ID NO: 1729.

The probes having the nucleotide sequence represented by SEQ ID NOS: 25022, 25023, 25024, 25025, 25026, 25027, 25028, 25029, 25030, 25031, 25032 were selected from the ORFs having the nucleotide sequence represented by SEQ ID NO: 1730.

The probes having the nucleotide sequence represented by SEQ ID NOS: 25033, 25034, 25035, 25036, 25037, 25038, 25039, 25040, 25041, 25042, 25043 were selected from the ORFs having the nucleotide sequence represented by SEQ ID NO: 1731.

The probes having the nucleotide sequence represented by SEQ ID NOS: 25044, 25045, 25046, 25047, 25048, 25049, 25050, 25051, 25052, 25053, 25054 were selected from the ORFs having the nucleotide sequence represented by SEQ ID NO: 1732.

The probes having the nucleotide sequence represented by SEQ ID NOS: 25055, 25056, 25057, 25058, 25059, 25060, 25061, 25062, 25063, 25064, 25065 were selected from the ORFs having the nucleotide sequence represented by SEQ ID NO: 1733.

The probes having the nucleotide sequence represented by SEQ ID NOS: 25066, 25067, 25068, 25069, 25070, 25071, 25072, 25073, 25074, 25075, 25076 were selected from the ORFs having the nucleotide sequence represented by SEQ ID NO: 1734.

The probes having the nucleotide sequence represented by SEQ ID NOS: 25077, 25078, 25079, 25080, 25081, 25082, 25083, 25084, 25085, 25086, 25087 were selected from the ORFs having the nucleotide sequence represented by SEQ ID NO: 1735.

The probes having the nucleotide sequence represented by SEQ ID NOS: 25088, 25089, 25090, 25091, 25092, 25093, 25094, 25095, 25096, 25097, 25098 were selected from the ORFs having the nucleotide sequence represented by SEQ ID NO: 1736.

The probes having the nucleotide sequence represented by SEQ ID NOS: 25099, 25100, 25101, 25102, 25103, 25104, 25105, 25106, 25107, 25108, 25109 were selected from the ORFs having the nucleotide sequence represented by SEQ ID NO: 1737.

The probes having the nucleotide sequence represented by SEQ ID NOS: 25110, 25111, 25112, 25113, 25114, 25115, 25116, 25117, 25118, 25119, 25120 were selected from the ORFs having the nucleotide sequence represented by SEQ ID NO: 1738.

The probes having the nucleotide sequence represented by SEQ ID NOS: 25121, 25122, 25123, 25124, 25125, 25126, 25127, 25128, 25129, 25130, 25131 were selected from the ORFs having the nucleotide sequence represented by SEQ ID NO: 1739.

The probes having the nucleotide sequence represented by SEQ ID NOS: 25132, 25133, 25134, 25135, 25136, 25137, 25138, 25139, 25140, 25141, 25142 were selected from the ORFs having the nucleotide sequence represented by SEQ ID NO: 1740.

The probes having the nucleotide sequence represented by SEQ ID NOS: 25143, 25144, 25145, 25146, 25147, 25148, 25149, 25150, 25151, 25152, 25153 were selected from the ORFs having the nucleotide sequence represented by SEQ ID NO: 1741.

The probes having the nucleotide sequence represented by SEQ ID NOS: 25154, 25155, 25156, 25157, 25158, 25159, 25160, 25161, 25162, 25163, 25164 were selected from the ORFs having the nucleotide sequence represented by SEQ ID NO: 1742.

The probes having the nucleotide sequence represented by SEQ ID NOS: 25165, 25166, 25167, 25168, 25169, 25170, 25171, 25172, 25173, 25174, 25175 were selected from the ORFs having the nucleotide sequence represented by SEQ ID NO: 1743.

The probes having the nucleotide sequence represented by SEQ ID NOS: 25176, 25177, 25178, 25179, 25180, 25181, 25182, 25183, 25184, 25185, 25186 were selected from the ORFs having the nucleotide sequence represented by SEQ ID NO: 1744.

The probes having the nucleotide sequence represented by SEQ ID NOS: 25187, 25188, 25189, 25190, 25191, 25192, 25193, 25194, 25195, 25196, 25197 were selected from the ORFs having the nucleotide sequence represented by SEQ ID NO: 1745.

The probes having the nucleotide sequence represented by SEQ ID NOS: 25198, 25199, 25200, 25201, 25202, 25203, 25204, 25205, 25206, 25207, 25208 were selected from the ORFs having the nucleotide sequence represented by SEQ ID NO: 1746.

The probes having the nucleotide sequence represented by SEQ ID NOS: 25209, 25210, 25211, 25212, 25213, 25214, 25215, 25216, 25217, 25218, 25219 were selected from the ORFs having the nucleotide sequence represented by SEQ ID NO: 1747.

The probes having the nucleotide sequence represented by SEQ ID NOS: 25220, 25221, 25222, 25223, 25224, 25225, 25226, 25227, 25228, 25229, 25230 were selected from the ORFs having the nucleotide sequence represented by SEQ ID NO: 1748.

The probes having the nucleotide sequence represented by SEQ ID NOS: 25231, 25232, 25233, 25234, 25235, 25236, 25237, 25238, 25239, 25240, 25241 were selected from the ORFs having the nucleotide sequence represented by SEQ ID NO: 1749.

The probes having the nucleotide sequence represented by SEQ ID NOS: 25242, 25243, 25244, 25245, 25246, 25247, 25248, 25249, 25250, 25251, 25252 were selected from the ORFs having the nucleotide sequence represented by SEQ ID NO: 1750.

The probes having the nucleotide sequence represented by SEQ ID NOS: 25253, 25254, 25255, 25256, 25257, 25258, 25259, 25260, 25261, 25262, 25263 were selected from the ORFs having the nucleotide sequence represented by SEQ ID NO: 1751.

The probes having the nucleotide sequence represented by SEQ ID NOS: 25264, 25265, 25266, 25267, 25268, 25269, 25270, 25271, 25272, 25273, 25274 were selected from the ORFs having the nucleotide sequence represented by SEQ ID NO: 1752.

The probes having the nucleotide sequence represented by SEQ ID NOS: 25275, 25276, 25277, 25278, 25279, 25280, 25281, 25282, 25283, 25284, 25285 were selected from the ORFs having the nucleotide sequence represented by SEQ ID NO: 1753.

The probes having the nucleotide sequence represented by SEQ ID NOS: 25286, 25287, 25288, 25289, 25290, 25291, 25292, 25293, 25294, 25295, 25296 were selected from the ORFs having the nucleotide sequence represented by SEQ ID NO: 1754.

The probes having the nucleotide sequence represented by SEQ ID NOS: 25297, 25298, 25299, 25300, 25301, 25302, 25303, 25304, 25305, 25306, 25307 were selected from the ORFs having the nucleotide sequence represented by SEQ ID NO: 1755.

The probes having the nucleotide sequence represented by SEQ ID NOS: 25308, 25309, 25310, 25311, 25312, 25313, 25314, 25315, 25316, 25317, 25318, 25319, 25320, 25321, 25322, 25323, 25324, 25325, 25326, 25327, 25328, 25329 were selected from the ORFs having the nucleotide sequence represented by SEQ ID NO: 1756.

The probes having the nucleotide sequence represented by SEQ ID NOS: 25330, 25331, 25332, 25333, 25334, 25335, 25336, 25337, 25338, 25339, 25340 were selected from the ORFs having the nucleotide sequence represented by SEQ ID NO: 1757.

The probes having the nucleotide sequence represented by SEQ ID NOS: 25341, 25342, 25343, 25344, 25345, 25346, 25347, 25348, 25349, 25350, 25351 were selected from the ORFs having the nucleotide sequence represented by SEQ ID NO: 1758.

The probes having the nucleotide sequence represented by SEQ ID NOS: 25352, 25353, 25354, 25355, 25356, 25357, 25358, 25359, 25360, 25361, 25362 were selected from the ORFs having the nucleotide sequence represented by SEQ ID NO: 1759.

The probes having the nucleotide sequence represented by SEQ ID NOS: 25363, 25364, 25365, 25366, 25367, 25368, 25369, 25370, 25371, 25372, 25373 were selected from the ORFs having the nucleotide sequence represented by SEQ ID NO: 1760.

The probes having the nucleotide sequence represented by SEQ ID NOS: 25374, 25375, 25376, 25377, 25378, 25379, 25380, 25381, 25382, 25383, 25384 were selected from the ORFs having the nucleotide sequence represented by SEQ ID NO: 1761.

The probes having the nucleotide sequence represented by SEQ ID NOS: 25385, 25386, 25387, 25388, 25389, 25390, 25391, 25392, 25393, 25394, 25395 were selected from the ORFs having the nucleotide sequence represented by SEQ ID NO: 1762.

The probes having the nucleotide sequence represented by SEQ ID NOS: 25396, 25397, 25398, 25399, 25400, 25401, 25402, 25403, 25404, 25405, 25406 were selected from the ORFs having the nucleotide sequence represented by SEQ ID NO: 1763.

The probes having the nucleotide sequence represented by SEQ ID NOS: 25407, 25408, 25409, 25410, 25411, 25412, 25413, 25414, 25415, 25416, 25417 were selected from the ORFs having the nucleotide sequence represented by SEQ ID NO: 1764.

The probes having the nucleotide sequence represented by SEQ ID NOS: 25418, 25419, 25420, 25421, 25422, 25423, 25424, 25425, 25426, 25427, 25428 were selected from the ORFs having the nucleotide sequence represented by SEQ ID NO: 1765.

The probes having the nucleotide sequence represented by SEQ ID NOS: 25429, 25430, 25431, 25432, 25433, 25434, 25435, 25436, 25437, 25438, 25439 were selected from the ORFs having the nucleotide sequence represented by SEQ ID NO: 1766.

The probes having the nucleotide sequence represented by SEQ ID NOS: 25440, 25441, 25442, 25443, 25444, 25445, 25446, 25447, 25448, 25449, 25450 were selected from the ORFs having the nucleotide sequence represented by SEQ ID NO: 1767.

The probes having the nucleotide sequence represented by SEQ ID NOS: 25451, 25452, 25453, 25454, 25455, 25456, 25457, 25458, 25459, 25460, 25461 were selected from the ORFs having the nucleotide sequence represented by SEQ ID NO: 1768.

The probes having the nucleotide sequence represented by SEQ ID NOS: 25462, 25463, 25464, 25465, 25466, 25467, 25468, 25469, 25470, 25471, 25472 were selected from the ORFs having the nucleotide sequence represented by SEQ ID NO: 1769.

The probes having the nucleotide sequence represented by SEQ ID NOS: 25473, 25474, 25475, 25476, 25477, 25478, 25479, 25480, 25481, 25482, 25483 were selected from the ORFs having the nucleotide sequence represented by SEQ ID NO: 1770.

The probes having the nucleotide sequence represented by SEQ ID NOS: 25484, 25485, 25486, 25487, 25488, 25489, 25490, 25491, 25492, 25493, 25494 were selected from the ORFs having the nucleotide sequence represented by SEQ ID NO: 1771.

The probes having the nucleotide sequence represented by SEQ ID NOS: 25495, 25496, 25497, 25498, 25499, 25500, 25501, 25502, 25503, 25504, 25505 were selected from the ORFs having the nucleotide sequence represented by SEQ ID NO: 1772.

The probes having the nucleotide sequence represented by SEQ ID NOS: 25506, 25507, 25508, 25509, 25510, 25511, 25512, 25513, 25514, 25515, 25516 were selected from the ORFs having the nucleotide sequence represented by SEQ ID NO: 1773.

The probes having the nucleotide sequence represented by SEQ ID NOS: 25517, 25518, 25519, 25520, 25521, 25522, 25523, 25524, 25525, 25526, 25527 were selected from the ORFs having the nucleotide sequence represented by SEQ ID NO: 1774.

The probes having the nucleotide sequence represented by SEQ ID NOS: 25528, 25529, 25530, 25531, 25532, 25533, 25534, 25535, 25536, 25537, 25538 were selected from the ORFs having the nucleotide sequence represented by SEQ ID NO: 1775.

The probes having the nucleotide sequence represented by SEQ ID NOS: 25539, 25540, 25541, 25542, 25543, 25544, 25545, 25546, 25547, 25548, 25549 were selected from the ORFs having the nucleotide sequence represented by SEQ ID NO: 1776.

The probes having the nucleotide sequence represented by SEQ ID NOS: 25550, 25551, 25552, 25553, 25554, 25555, 25556, 25557, 25558, 25559, 25560 were selected from the ORFs having the nucleotide sequence represented by SEQ ID NO: 1777.

The probes having the nucleotide sequence represented by SEQ ID NOS: 25561, 25562, 25563, 25564, 25565, 25566, 25567, 25568, 25569, 25570, 25571 were selected from the ORFs having the nucleotide sequence represented by SEQ ID NO: 1778.

The probes having the nucleotide sequence represented by SEQ ID NOS: 25572, 25573, 25574, 25575, 25576, 25577, 25578, 25579, 25580, 25581, 25582 were selected from the ORFs having the nucleotide sequence represented by SEQ ID NO: 1779.

The probes having the nucleotide sequence represented by SEQ ID NOS: 25583, 25584, 25585, 25586, 25587, 25588, 25589, 25590, 25591, 25592, 25593 were selected from the ORFs having the nucleotide sequence represented by SEQ ID NO: 1780.

The probes having the nucleotide sequence represented by SEQ ID NOS: 25594, 25595, 25596, 25597, 25598, 25599, 25600, 25601, 25602, 25603, 25604 were selected from the ORFs having the nucleotide sequence represented by SEQ ID NO: 1781.

The probes having the nucleotide sequence represented by SEQ ID NOS: 25605, 25606, 25607, 25608, 25609, 25610, 25611, 25612, 25613, 25614, 25615 were selected from the ORFs having the nucleotide sequence represented by SEQ ID NO: 1782.

The probes having the nucleotide sequence represented by SEQ ID NOS: 25616, 25617, 25618, 25619, 25620, 25621, 25622, 25623, 25624, 25625, 25626 were selected from the ORFs having the nucleotide sequence represented by SEQ ID NO: 1783.

The probes having the nucleotide sequence represented by SEQ ID NOS: 25627, 25628, 25629, 25630, 25631, 25632, 25633, 25634, 25635, 25636, 25637 were selected from the ORFs having the nucleotide sequence represented by SEQ ID NO: 1784.

The probes having the nucleotide sequence represented by SEQ ID NOS: 25638, 25639, 25640, 25641, 25642, 25643, 25644, 25645, 25646, 25647, 25648 were selected from the ORFs having the nucleotide sequence represented by SEQ ID NO: 1785.

The probes having the nucleotide sequence represented by SEQ ID NOS: 25649, 25650, 25651, 25652, 25653, 25654, 25655, 25656, 25657, 25658, 25659 were selected from the ORFs having the nucleotide sequence represented by SEQ ID NO: 1786.

The probes having the nucleotide sequence represented by SEQ ID NOS: 25660, 25661, 25662, 25663, 25664, 25665, 25666, 25667, 25668, 25669, 25670 were selected from the ORFs having the nucleotide sequence represented by SEQ ID NO: 1787.

The probes having the nucleotide sequence represented by SEQ ID NOS: 25671, 25672, 25673, 25674, 25675, 25676, 25677, 25678, 25679, 25680, 25681 were selected from the ORFs having the nucleotide sequence represented by SEQ ID NO: 1788.

The probes having the nucleotide sequence represented by SEQ ID NOS: 25682, 25683, 25684, 25685, 25686, 25687, 25688, 25689, 25690, 25691, 25692 were selected from the ORFs having the nucleotide sequence represented by SEQ ID NO: 1789.

The probes having the nucleotide sequence represented by SEQ ID NOS: 25693, 25694, 25695, 25696, 25697, 25698, 25699, 25700, 25701, 25702, 25703 were selected from the ORFs having the nucleotide sequence represented by SEQ ID NO: 1790.

The probes having the nucleotide sequence represented by SEQ ID NOS: 25704, 25705, 25706, 25707, 25708, 25709, 25710, 25711, 25712, 25713, 25714 were selected from the ORFs having the nucleotide sequence represented by SEQ ID NO: 1791.

The probes having the nucleotide sequence represented by SEQ ID NOS: 25715, 25716, 25717, 25718, 25719, 25720, 25721, 25722, 25723, 25724, 25725 were selected from the ORFs having the nucleotide sequence represented by SEQ ID NO: 1792.

The probes having the nucleotide sequence represented by SEQ ID NOS: 25726, 25727, 25728, 25729, 25730, 25731, 25732, 25733, 25734, 25735, 25736 were selected from the ORFs having the nucleotide sequence represented by SEQ ID NO: 1793.

The probes having the nucleotide sequence represented by SEQ ID NOS: 25737, 25738, 25739, 25740, 25741, 25742, 25743, 25744, 25745, 25746, 25747, 25748, 25749, 25750, 25751, 25752, 25753, 25754, 25755, 25756, 25757, 25758 were selected from the ORFs having the nucleotide sequence represented by SEQ ID NO: 1794.

The probes having the nucleotide sequence represented by SEQ ID NOS: 25759, 25760, 25761, 25762, 25763, 25764, 25765, 25766, 25767, 25768, 25769 were selected from the ORFs having the nucleotide sequence represented by SEQ ID NO: 1795.

The probes having the nucleotide sequence represented by SEQ ID NOS: 25770, 25771, 25772, 25773, 25774, 25775, 25776, 25777, 25778, 25779, 25780 were selected from the ORFs having the nucleotide sequence represented by SEQ ID NO: 1796.

The probes having the nucleotide sequence represented by SEQ ID NOS: 25781, 25782, 257.83, 25784, 25785, 25786, 25787, 25788, 25789, 25790, 25791 were selected from the ORFs having the nucleotide sequence represented by SEQ ID NO: 1797.

The probes having the nucleotide sequence represented by SEQ ID NOS: 25792, 25793, 25794, 25795, 25796, 25797, 25798, 25799, 25800, 25801, 25802 were selected from the ORFs having the nucleotide sequence represented by SEQ ID NO: 1798.

The probes having the nucleotide sequence represented by SEQ ID NOS: 25803, 25804, 25805, 25806, 25807, 25808, 25809, 25810, 25811, 25812, 25813 were selected from the ORFs having the nucleotide sequence represented by SEQ ID NO: 1799.

The probes having the nucleotide sequence represented by SEQ ID NOS: 25814, 25815, 25816, 25817, 25818, 25819, 25820, 25821, 25822, 25823, 25824 were selected from the ORFs having the nucleotide sequence represented by SEQ ID NO: 1800.

The probes having the nucleotide sequence represented by SEQ ID NOS: 25825, 25826, 25827, 25828, 25829, 25830, 25831, 25832, 25833, 25834, 25835 were selected from the ORFs having the nucleotide sequence represented by SEQ ID NO: 1801.

The probes having the nucleotide sequence represented by SEQ ID NOS: 25836, 25837, 25838, 25839, 25840, 25841, 25842, 25843, 25844, 25845, 25846 were selected from the ORFs having the nucleotide sequence represented by SEQ ID NO: 1802.

The probes having the nucleotide sequence represented by SEQ ID NOS: 25847, 25848, 25849, 25850, 25851, 25852, 25853, 25854, 25855, 25856, 25857 were selected from the ORFs having the nucleotide sequence represented by SEQ ID NO: 1803.

The probes having the nucleotide sequence represented by SEQ ID NOS: 25858, 25859, 25860, 25861, 25862, 25863, 25864, 25865, 25866, 25867, 25868 were selected from the ORFs having the nucleotide sequence represented by SEQ ID NO: 1804.

The probes having the nucleotide sequence represented by SEQ ID NOS: 25869, 25870, 25871, 25872, 25873, 25874, 25875, 25876, 25877, 25878, 25879 were selected from the ORFs having the nucleotide sequence represented by SEQ ID NO: 1805.

The probes having the nucleotide sequence represented by SEQ ID NOS: 25880, 25881, 25882, 25883, 25884, 25885, 25886, 25887, 25888, 25889, 25890 were selected from the ORFs having the nucleotide sequence represented by SEQ ID NO: 1806.

The probes having the nucleotide sequence represented by SEQ ID NOS: 25891, 25892, 25893, 25894, 25895, 25896, 25897, 25898, 25899, 25900, 25901 were selected from the ORFs having the nucleotide sequence represented by SEQ ID NO: 1807.

The probes having the nucleotide sequence represented by SEQ ID NOS: 25902, 25903, 25904, 25905, 25906, 25907, 25908, 25909, 25910, 25911, 25912 were selected from the ORFs having the nucleotide sequence represented by SEQ ID NO: 1808.

The probes having the nucleotide sequence represented by SEQ ID NOS: 25913, 25914, 25915, 25916, 25917, 25918, 25919, 25920, 25921, 25922, 25923 were selected from the ORFs having the nucleotide sequence represented by SEQ ID NO: 1809.

The probes having the nucleotide sequence represented by SEQ ID NOS: 25924, 25925, 25926, 25927, 25928, 25929, 25930, 25931, 25932, 25933, 25934 were selected from the ORFs having the nucleotide sequence represented by SEQ ID NO: 1810.

The probes having the nucleotide sequence represented by SEQ ID NOS: 25935, 25936, 25937, 25938, 25939, 25940, 25941, 25942, 25943, 25944, 25945 were selected from the ORFs having the nucleotide sequence represented by SEQ ID NO: 1811.

The probes having the nucleotide sequence represented by SEQ ID NOS: 25946, 25947, 25948, 25949, 25950, 25951, 25952, 25953, 25954, 25955, 25956 were selected from the ORFs having the nucleotide sequence represented by SEQ ID NO: 1812.

The probes having the nucleotide sequence represented by SEQ ID NOS: 25957, 25958, 25959, 25960, 25961, 25962, 25963, 25964, 25965, 25966, 25967 were selected from the ORFs having the nucleotide sequence represented by SEQ ID NO: 1813.

The probes having the nucleotide sequence represented by SEQ ID NOS: 25968, 25969, 25970, 25971, 25972, 25973, 25974, 25975, 25976, 25977, 25978 were selected from the ORFs having the nucleotide sequence represented by SEQ ID NO: 1814.

The probes having the nucleotide sequence represented by SEQ ID NOS: 25979, 25980, 25981, 25982, 25983, 25984, 25985, 25986, 25987, 25988, 25989 were selected from the ORFs having the nucleotide sequence represented by SEQ ID NO: 1815.

The probes having the nucleotide sequence represented by SEQ ID NOS: 25990, 25991, 25992, 25993, 25994, 25995, 25996, 25997, 25998, 25999, 26000 were selected from the ORFs having the nucleotide sequence represented by SEQ ID NO: 1816.

The probes having the nucleotide sequence represented by SEQ ID NOS: 26001, 26002, 26003, 26004, 26005, 26006, 26007, 26008, 26009, 26010, 26011 were selected from the ORFs having the nucleotide sequence represented by SEQ ID NO: 1817.

The probes having the nucleotide sequence represented by SEQ ID NOS: 26012, 26013, 26014, 26015, 26016, 26017, 26018, 26019, 26020, 26021, 26022 were selected from the ORFs having the nucleotide sequence represented by SEQ ID NO: 1818.

The probes having the nucleotide sequence represented by SEQ ID NOS: 26023, 26024, 26025, 26026, 26027, 26028, 26029, 26030, 26031, 26032, 26033 were selected from the ORFs having the nucleotide sequence represented by SEQ ID NO: 1819.

The probes having the nucleotide sequence represented by SEQ ID NOS: 26034, 26035, 26036, 26037, 26038, 26039, 26040, 26041, 26042, 26043, 26044 were selected from the ORFs having the nucleotide sequence represented by SEQ ID NO: 1820.

The probes having the nucleotide sequence represented by SEQ ID NOS: 26045, 26046, 26047, 26048, 26049, 26050, 26051, 260.52, 26053, 26054, 26055 were selected from the ORFs having the nucleotide sequence represented by SEQ ID NO: 1821.

The probes having the nucleotide sequence represented by SEQ ID NOS: 26056, 26057, 26058, 26059, 26060, 26061, 26062, 26063, 26064, 26065, 26066 were selected from the ORFs having the nucleotide sequence represented by SEQ ID NO: 1822.

The probes having the nucleotide sequence represented by SEQ ID NOS: 26067, 26068, 26069, 26070, 26071, 26072, 26073, 26074, 26075, 26076, 26077 were selected from the ORFs having the nucleotide sequence represented by SEQ ID NO: 1823.

The probes having the nucleotide sequence represented by SEQ ID NOS: 26078, 26079, 26080, 26081, 2.6082, 26083, 26084, 26085, 26086, 26087, 26088 were selected from the ORFs having the nucleotide sequence represented by SEQ ID NO: 1824.

The probes having the nucleotide sequence represented by SEQ ID NOS: 26089, 26090, 26091, 26092, 26093, 26094, 26095, 26096, 26097, 26098, 26099 were selected from the ORFs having the nucleotide sequence represented by SEQ ID NO: 1825.

The probes having the nucleotide sequence represented by SEQ ID NOS: 26100, 26101, 26102, 26103, 26104, 26105, 26106, 26107, 26108, 26109, 26110 were selected from the ORFs having the nucleotide sequence represented by SEQ ID NO: 1826.

The probes having the nucleotide sequence represented by SEQ ID NOS: 26111, 26112, 26113, 26114, 26115, 26116, 26117, 26118, 26119, 26120, 26121 were selected from the ORFs having the nucleotide sequence represented by SEQ ID NO: 1827.

The probes having the nucleotide sequence represented by SEQ ID NOS: 26122, 26123, 26124, 26125, 26126, 26127, 26128, 26129, 26130, 26131, 26132 were selected from the ORFs having the nucleotide sequence represented by SEQ ID NO: 1828.

The probes having the nucleotide sequence represented by SEQ ID NOS: 26133, 26134, 26135, 26136, 26137, 26138, 26139, 26140, 26141, 26142, 26143 were selected from the ORFs having the nucleotide sequence represented by SEQ ID NO: 1829.

The probes having the nucleotide sequence represented by SEQ ID NOS: 26144, 26145, 26146, 26147, 26148, 26149, 26150, 26151, 26152, 26153, 26154 were selected from the ORFs having the nucleotide sequence represented by SEQ ID NO: 1830.

The probes having the nucleotide sequence represented by SEQ ID NOS: 26155, 26156, 26157, 26158, 26159, 26160, 26161, 26162, 26163, 26164, 26165 were selected from the ORFs having the nucleotide sequence represented by SEQ ID NO: 1831.

The probes having the nucleotide sequence represented by SEQ ID NOS: 26166, 26167, 26168, 26169, 26170, 26171, 26172, 26173, 26174, 26175, 26176 were selected from the ORFs having the nucleotide sequence represented by SEQ ID NO: 1832.

The probes having the nucleotide sequence represented by SEQ ID NOS: 26177, 26178, 26179, 26180, 26181, 26182, 26183, 26184, 26185, 26186, 26187 were selected from the ORFs having the nucleotide sequence represented by SEQ ID NO: 1833.

The probes having the nucleotide sequence represented by SEQ ID NOS: 26188, 26189, 26190, 26191, 26192, 26193, 26194, 26195, 26196, 26197, 26198 were selected from the ORFs having the nucleotide sequence represented by SEQ ID NO: 1834.

The probes having the nucleotide sequence represented by SEQ ID NOS: 26199, 26200, 26201, 26202, 26203, 26204, 26205, 26206, 26207, 26208, 26209 were selected from the ORFs having the nucleotide sequence represented by SEQ ID NO: 1835.

The probes having the nucleotide sequence represented by SEQ ID NOS: 26210, 26211, 26212, 26213, 26214, 26215, 26216, 26217, 26218, 26219, 26220 were selected from the ORFs having the nucleotide sequence represented by SEQ ID NO: 1836.

The probes having the nucleotide sequence represented by SEQ ID NOS: 26221, 26222, 26223, 26224, 26225, 26226, 26227, 26228, 26229, 26230, 26231 were selected from the ORFs having the nucleotide sequence represented by SEQ ID NO: 1837.

The probes having the nucleotide sequence represented by SEQ ID NOS: 26232, 26233, 26234, 26235, 26236, 26237, 26238, 26239, 26240, 26241, 26242 were selected from the ORFs having the nucleotide sequence represented by SEQ ID NO: 1838.

The probes having the nucleotide sequence represented by SEQ ID NOS: 26243, 26244, 26245, 26246, 26247, 26248, 26249, 26250, 26251, 26252, 26253 were selected from the ORFs having the nucleotide sequence represented by SEQ ID NO: 1839.

The probes having the nucleotide sequence represented by SEQ ID NOS: 26254, 26255, 26256, 26257, 26258, 26259, 26260, 26261, 26262, 26263, 26264 were selected from the ORFs having the nucleotide sequence represented by SEQ ID NO: 1840.

The probes having the nucleotide sequence represented by SEQ ID NOS: 26265, 26266, 26267, 26268, 26269, 26270, 26271, 26272, 26273, 26274, 26275 were selected from the ORFs having the nucleotide sequence represented by SEQ ID NO: 1841.

The probes having the nucleotide sequence represented by SEQ ID NOS: 26276, 26277, 26278, 26279, 26280, 26281, 26282, 26283, 26284, 26285, 26286 were selected from the ORFs having the nucleotide sequence represented by SEQ ID NO: 1842.

The probes having the nucleotide sequence represented by SEQ ID NOS: 26287, 26288, 26289, 26290, 26291, 26292, 26293, 26294, 26295, 26296, 26297 were selected from the ORFs having the nucleotide sequence represented by SEQ ID NO: 1843.

The probes having the nucleotide sequence represented by SEQ ID NOS: 26298, 26299, 26300, 26301, 26302, 26303, 26304, 26305, 26306, 26307, 26308 were selected from the ORFs having the nucleotide sequence represented by SEQ ID NO: 1844.

The probes having the nucleotide sequence represented by SEQ ID NOS: 26309, 26310, 26311, 26312, 26313, 26314, 26315, 26316, 26317, 26318, 26319 were selected from the ORFs having the nucleotide sequence represented by SEQ ID NO: 1845.

The probes having the nucleotide sequence represented by SEQ ID NOS: 26320, 26321, 26322, 26323, 26324, 26325, 26326, 26327, 26328, 26329, 26330 were selected from the ORFs having the nucleotide sequence represented by SEQ ID NO: 1846.

The probes having the nucleotide sequence represented by SEQ ID NOS: 26331, 26332, 26333, 26334, 26335, 26336, 26337, 26338, 26339, 26340, 26341 were selected from the ORFs having the nucleotide sequence represented by SEQ ID NO: 1847.

The probes having the nucleotide sequence represented by SEQ ID NOS: 26342, 26343, 26344, 26345, 26346, 26347, 26348, 26349, 26350, 26351, 26352 were selected from the ORFs having the nucleotide sequence represented by SEQ ID NO: 1848.

The probes having the nucleotide sequence represented by SEQ ID NOS: 26353, 26354, 26355, 26356, 26357, 26358, 26359, 26360, 26361, 26362, 26363 were selected from the ORFs having the nucleotide sequence represented by SEQ ID NO: 1849.

The probes having the nucleotide sequence represented by SEQ ID NOS: 26364, 26365, 26366, 26367, 26368, 26369, 26370, 26371, 26372, 26373, 26374 were selected from the ORFs having the nucleotide sequence represented by SEQ ID NO: 1850.

The probes having the nucleotide sequence represented by SEQ ID NOS: 26375, 26376, 26377, 26378, 26379, 26380, 26381, 26382, 26383, 26384, 26385 were selected from the ORFs having the nucleotide sequence represented by SEQ ID NO: 1851.

The probes having the nucleotide sequence represented by SEQ ID NOS: 26386, 26387, 26388, 26389, 26390, 26391, 26392, 26393, 26394, 26395, 26396 were selected from the ORFs having the nucleotide sequence represented by SEQ ID NO: 1852.

The probes having the nucleotide sequence represented by SEQ ID NOS: 26397, 26398, 26399, 26400, 26401, 26402, 26403, 26404, 26405, 26406, 26407 were selected from the ORFs having the nucleotide sequence represented by SEQ ID NO: 1853.

The probes having the nucleotide sequence represented by SEQ ID NOS: 26408, 26409, 26410, 26411, 26412, 26413, 26414, 26415, 26416, 26417, 26418 were selected from the ORFs having the nucleotide sequence represented by SEQ ID NO: 1854.

The probes having the nucleotide sequence represented by SEQ ID NOS: 26419, 26420, 26421, 26422, 26423, 26424, 26425, 26426, 26427, 26428, 26429 were selected from the ORFs having the nucleotide sequence represented by SEQ ID NO: 1855.

The probes having the nucleotide sequence represented by SEQ ID NOS: 26430, 26431, 26432, 26433, 26434, 26435, 26436, 26437, 26438, 26439, 26440 were selected from the ORFs having the nucleotide sequence represented by SEQ ID NO: 1856.

The probes having the nucleotide sequence represented by SEQ ID NOS: 26441, 26442, 26443, 26444, 26445, 26446, 26447, 26448, 26449, 26450, 26451 were selected from the ORFs having the nucleotide sequence represented by SEQ ID NO: 1857.

The probes having the nucleotide sequence represented by SEQ ID NOS: 26452, 26453, 26454, 26455, 26456, 26457, 26458, 26459, 26460, 26461, 26462 were selected from the ORFs having the nucleotide sequence represented by SEQ ID NO: 1858.

The probes having the nucleotide sequence represented by SEQ ID NOS: 26463, 26464, 26465, 26466, 26467, 26468, 26469, 26470, 26471, 26472, 26473 were selected from the ORFs having the nucleotide sequence represented by SEQ ID NO: 1859.

The probes having the nucleotide sequence represented by SEQ ID NOS: 26474, 26475, 26476, 26477, 26478, 26479, 26480, 26481, 26482, 26483, 26484 were selected from the ORFs having the nucleotide sequence represented by SEQ ID NO: 1860.

The probes having the nucleotide sequence represented by SEQ ID NOS: 26485, 26486, 26487, 26488, 26489, 26490, 26491, 26492, 26493, 26494, 26495 were selected from the ORFs having the nucleotide sequence represented by SEQ ID NO: 1861.

The probes having the nucleotide sequence represented by SEQ ID NOS: 26496, 26497, 26498, 26499, 26500, 26501, 26502, 26503, 26504, 26505, 26506 were selected from the ORFs having the nucleotide sequence represented by SEQ ID NO: 1862.

The probes having the nucleotide sequence represented by SEQ ID NOS: 26507, 26508, 26509, 26510, 26511, 26512, 26513, 26514, 26515, 26516, 26517 were selected from the ORFs having the nucleotide sequence represented by SEQ ID NO: 1863.

The probes having the nucleotide sequence represented by SEQ ID NOS: 26518, 26519, 26520, 26521, 26522, 26523, 26524, 26525, 26526, 26527, 26528 were selected from the ORFs having the nucleotide sequence represented by SEQ ID NO: 1864.

The probes having the nucleotide sequence represented by SEQ ID NOS: 26529, 26530, 26531, 26532, 26533, 26534, 26535, 26536, 26537, 26538, 26539 were selected from the ORFs having the nucleotide sequence represented by SEQ ID NO: 1865.

The probes having the nucleotide sequence represented by SEQ ID NOS: 26540, 26541, 26542, 26543, 26544, 26545, 26546, 26547, 26548, 26549, 26550 were selected from the ORFs having the nucleotide sequence represented by SEQ ID NO: 1866.

The probes having the nucleotide sequence represented by SEQ ID NOS: 26551, 26552, 26553, 26554, 26555, 26556, 26557, 26558, 26559, 26560, 26561 were selected from the ORFs having the nucleotide sequence represented by SEQ ID NO: 1867.

The probes having the nucleotide sequence represented by SEQ ID NOS: 26562, 26563, 26564, 26565, 26566, 26567, 26568, 26569, 26570, 26571, 26572 were selected from the ORFs having the nucleotide sequence represented by SEQ ID NO: 1868.

The probes having the nucleotide sequence represented by SEQ ID NOS: 26573, 26574, 26575, 26576, 26577, 26578, 26579, 26580, 26581, 26582, 26583 were selected from the ORFs having the nucleotide sequence represented by SEQ ID NO: 1869.

The probes having the nucleotide sequence represented by SEQ ID NOS: 26584, 26585, 26586, 26587, 26588, 26589, 26590, 26591, 26592, 26593, 26594 were selected from the ORFs having the nucleotide sequence represented by SEQ ID NO: 1870.

The probes having the nucleotide sequence represented by SEQ ID NOS: 26595, 26596, 26597, 26598, 26599, 26600, 26601, 26602, 26603, 26604, 26605 were selected from the ORFs having the nucleotide sequence represented by SEQ ID NO: 1871.

The probes having the nucleotide sequence represented by SEQ ID NOS: 26606, 26607, 26608, 26609, 26610, 26611, 26612, 26613, 26614, 26615, 26616 were selected from the ORFs having the nucleotide sequence represented by SEQ ID NO: 1872.

The probes having the nucleotide sequence represented by SEQ ID NOS: 26617, 26618, 26619, 26620, 26621, 26622, 26623, 26624, 26625, 26626, 26627 were selected from the ORFs having the nucleotide sequence represented by SEQ ID NO: 1873.

The probes having the nucleotide sequence represented by SEQ ID NOS: 26628, 26629, 26630, 26631, 26632, 26633, 26634, 26635, 26636, 26637, 26638 were selected from the ORFs having the nucleotide sequence represented by SEQ ID NO: 1874.

The probes having the nucleotide sequence represented by SEQ ID NOS: 26639, 26640, 26641, 26642, 26643, 26644, 26645, 26646, 26647, 26648, 26649 were selected from the ORFs having the nucleotide sequence represented by SEQ ID NO: 1875.

The probes having the nucleotide sequence represented by SEQ ID NOS: 26650, 26651, 26652, 26653, 26654, 26655, 26656, 26657, 26658, 26659, 26660 were selected from the ORFs having the nucleotide sequence represented by SEQ ID NO: 1876.

The probes having the nucleotide sequence represented by SEQ ID NOS: 26661, 26662, 26663, 26664, 26665, 26666, 26667, 26668, 26669, 26670, 26671 were selected from the ORFs having the nucleotide sequence represented by SEQ ID NO: 1877.

The probes having the nucleotide sequence represented by SEQ ID NOS: 26672, 26673, 26674, 26675, 26676, 26677, 26678, 26679, 26680, 26681, 26682 were selected from the ORFs having the nucleotide sequence represented by SEQ ID NO: 1878.

The probes having the nucleotide sequence represented by SEQ ID NOS: 26683, 26684, 26685, 26686, 26687, 26688, 26689, 26690, 26691, 26692, 26693, 26694, 26695, 26696, 26697, 26698, 26699, 26700, 26701, 26702, 26703, 26704 were selected from the ORFs having the nucleotide sequence represented by SEQ ID NO: 1879.

The probes having the nucleotide sequence represented by SEQ ID NOS: 26705, 26706, 26707, 26708, 26709, 26710, 26711, 26712, 26713, 26714, 26715 were selected from the ORFs having the nucleotide sequence represented by SEQ ID NO: 1880.

The probes having the nucleotide sequence represented by SEQ ID NOS: 26716, 26717, 26718, 26719, 26720, 26721, 26722, 26723, 26724, 26725, 26726 were selected from the ORFs having the nucleotide sequence represented by SEQ ID NO: 1881.

The probes having the nucleotide sequence represented by SEQ ID NOS: 26727, 26728, 26729, 26730, 26731, 26732, 26733, 26734, 26735, 26736, 26737 were selected from the ORFs having the nucleotide sequence represented by SEQ ID NO: 1882.

The probes having the nucleotide sequence represented by SEQ ID NOS: 26738, 26739, 26740, 26741, 26742, 26743, 26744, 26745, 26746, 26747, 26748 were selected from the ORFs having the nucleotide sequence represented by SEQ ID NO: 1883.

The probes having the nucleotide sequence represented by SEQ ID NOS: 26749, 26750, 26751, 26752, 26753, 26754, 26755, 26756, 26757, 26758, 26759 were selected from the ORFs having the nucleotide sequence represented by SEQ ID NO: 1884.

The probes having the nucleotide sequence represented by SEQ ID NOS: 26760, 26761, 26762, 26763, 26764, 26765, 26766, 26767, 26768, 26769, 26770 were selected from the ORFs having the nucleotide sequence represented by SEQ ID NO: 1885.

The probes having the nucleotide sequence represented by SEQ ID NOS: 26771, 26772, 26773, 26774, 26775, 26776, 26777, 26778, 26779, 26780, 26781 were selected from the ORFs having the nucleotide sequence represented by SEQ ID NO: 1886.

The probes having the nucleotide sequence represented by SEQ ID NOS: 26782, 26783, 26784, 26785, 26786, 26787, 26788, 26789, 26790, 26791, 26792 were selected from the ORFs having the nucleotide sequence represented by SEQ ID NO: 1887.

The probes having the nucleotide sequence represented by SEQ ID NOS: 26793, 26794, 26795, 26796, 26797, 26798, 26799, 26800, 26801, 26802, 26803 were selected from the ORFs having the nucleotide sequence represented by SEQ ID NO: 1888.

The probes having the nucleotide sequence represented by SEQ ID NOS: 26804, 26805, 26806, 26807, 26808, 26809, 26810, 26811, 26812, 26813, 26814 were selected from the ORFs having the nucleotide sequence represented by SEQ ID NO: 1889.

The probes having the nucleotide sequence represented by SEQ ID NOS: 26815, 26816, 26817, 26818, 26819, 26820, 26821, 26822, 26823, 26824, 26825 were selected from the ORFs having the nucleotide sequence represented by SEQ ID NO: 1890.

The probes having the nucleotide sequence represented by SEQ ID NOS: 26826, 26827, 26828, 26829, 26830, 26831, 26832, 26833, 26834, 26835, 26836 were selected from the ORFs having the nucleotide sequence represented by SEQ ID NO: 1891.

The probes having the nucleotide sequence represented by SEQ ID NOS: 26837, 26838, 26839, 26840, 26841, 26842, 26843, 26844, 26845, 26846, 26847 were selected from the ORFs having the nucleotide sequence represented by SEQ ID NO: 1892.

The probes having the nucleotide sequence represented by SEQ ID NOS: 26848, 26849, 26850, 26851, 26852, 26853, 26854, 26855, 26856, 26857, 26858 were selected from the ORFs having the nucleotide sequence represented by SEQ ID NO: 1893.

The probes having the nucleotide sequence represented by SEQ ID NOS: 26859, 26860, 26861, 26862, 26863, 26864, 26865, 26866, 26867, 26868, 26869 were selected from the ORFs having the nucleotide sequence represented by SEQ ID NO: 1894.

The probes having the nucleotide sequence represented by SEQ ID NOS: 26870, 26871, 26872, 26873, 26874, 26875, 26876, 26877, 26878, 26879, 26880 were selected from the ORFs having the nucleotide sequence represented by SEQ ID NO: 1895.

The probes having the nucleotide sequence represented by SEQ ID NOS: 26881, 26882, 26883, 26884, 26885, 26886, 26887, 26888, 26889, 26890, 26891 were selected from the ORFs having the nucleotide sequence represented by SEQ ID NO: 1896.

The probes having the nucleotide sequence represented by SEQ ID NOS: 26892, 26893, 26894, 26895, 26896, 26897, 26898, 26899, 26900, 26901, 26902, 26903, 26904, 26905, 26906, 26907, 26908, 26909, 26910, 26911, 26912, 26913 were selected from the ORFs having the nucleotide sequence represented by SEQ ID NO: 1897.

The probes having the nucleotide sequence represented by SEQ ID NOS: 26914, 26915, 26916, 26917, 26918, 26919, 26920, 26921, 26922, 26923, 26924 were selected from the ORFs having the nucleotide sequence represented by SEQ ID NO: 1898.

The probes having the nucleotide sequence represented by SEQ ID NOS: 26925, 26926, 26927, 26928, 26929, 26930, 26931, 26932, 26933, 26934, 26935 were selected from the ORFs having the nucleotide sequence represented by SEQ ID NO: 1899.

The probes having the nucleotide sequence represented by SEQ ID NOS: 26936, 26937, 26938, 26939, 26940, 26941, 26942, 26943, 26944, 26945, 26946 were selected from the ORFs having the nucleotide sequence represented by SEQ ID NO: 1900.

The probes having the nucleotide sequence represented by SEQ ID NOS: 26947, 26948, 26949, 26950, 26951, 26952, 26953, 26954, 26955, 26956, 26957 were selected from the ORFs having the nucleotide sequence represented by SEQ ID NO: 1901.

The probes having the nucleotide sequence represented by SEQ ID NOS: 26958, 26959, 26960, 26961, 26962, 26963, 26964, 26965, 26966, 26967, 26968 were selected from the ORFs having the nucleotide sequence represented by SEQ ID NO: 1902.

The probes having the nucleotide sequence represented by SEQ ID NOS: 26969, 26970, 26971, 26972, 26973, 26974, 26975, 26976, 26977, 26978, 26979 were selected from the ORFs having the nucleotide sequence represented by SEQ ID NO: 1903.

The probes having the nucleotide sequence represented by SEQ ID NOS: 26980, 26981, 26982, 26983, 26984, 26985, 26986, 26987, 26988, 26989, 26990 were selected from the ORFs having the nucleotide sequence represented by SEQ ID NO: 1904.

The probes having the nucleotide sequence represented by SEQ ID NOS: 26991, 26992, 26993, 26994, 26995, 26996, 26997, 26998, 26999, 27000, 27001 were selected from the ORFs having the nucleotide sequence represented by SEQ ID NO: 1905.

The probes having the nucleotide sequence represented by SEQ ID NOS: 27002, 27003, 27004, 27005, 27006, 27007, 27008, 27009, 27010, 27011, 27012 were selected from the ORFs having the nucleotide sequence represented by SEQ ID NO: 1906.

The probes having the nucleotide sequence represented by SEQ ID NOS: 27013, 27014, 27015, 27016, 27017, 27018, 27019, 27020, 27021, 27022, 27023 were selected from the ORFs having the nucleotide sequence represented by SEQ ID NO: 1907.

The probes having the nucleotide sequence represented by SEQ ID NOS: 27024, 27025, 27026, 27027, 27028, 27029, 27030, 27031, 27032, 27033, 27034 were selected from the ORFs having the nucleotide sequence represented by SEQ ID NO: 1908.

The probes having the nucleotide sequence represented by SEQ ID NOS: 27035, 27036, 27037, 27038, 27039, 27040, 27041, 27042, 27043, 27044, 27045 were selected from the ORFs having the nucleotide sequence represented by SEQ ID NO: 1909.

The probes having the nucleotide sequence represented by SEQ ID NOS: 27046, 27047, 27048, 27049, 27050, 27051, 27052, 27053, 27054, 27055, 27056 were selected from the ORFs having the nucleotide sequence represented by SEQ ID NO: 1910.

The probes having the nucleotide sequence represented by SEQ ID NOS: 27057, 27058, 27059, 27060, 27061, 27062, 27063, 27064, 27065, 27066, 27067 were selected from the ORFs having the nucleotide sequence represented by SEQ ID NO: 1911.

The probes having the nucleotide sequence represented by SEQ ID NOS: 27068, 27069, 27070, 27071, 27072, 27073, 27074, 27075, 27076, 27077, 27078 were selected from the ORFs having the nucleotide sequence represented by SEQ ID NO: 1912.

The probes having the nucleotide sequence represented by SEQ ID NOS: 27079, 27080, 27081, 27082, 27083, 27084, 27085, 27086, 27087, 27088, 27089 were selected from the ORFs having the nucleotide sequence represented by SEQ ID NO: 1913.

The probes having the nucleotide sequence represented by SEQ ID NOS: 27090, 27091, 27092, 27093, 27094, 27095, 27096, 27097, 27098, 27099, 27100 were selected from the ORFs having the nucleotide sequence represented by SEQ ID NO: 1914.

The probes having the nucleotide sequence represented by SEQ ID NOS: 27101, 27102, 27103, 27104, 27105, 27106, 27107, 27108, 27109, 27110, 27111 were selected from the ORFs having the nucleotide sequence represented by SEQ ID NO: 1915.

The probes having the nucleotide sequence represented by SEQ ID NOS: 27112, 27113, 27114, 27115, 27116, 27117, 27118, 27119, 27120, 27121, 27122 were selected from the ORFs having the nucleotide sequence represented by SEQ ID NO: 1916.

The probes having the nucleotide sequence represented by SEQ ID NOS: 27123, 27124, 27125, 27126, 27127, 27128, 27129, 27130, 27131, 27132, 27133 were selected from the ORFs having the nucleotide sequence represented by SEQ ID NO: 1917.

The probes having the nucleotide sequence represented by SEQ ID NOS: 27134, 27135, 27136, 27137, 27138, 27139, 27140, 27141, 27142, 27143, 27144, 27145, 27146, 27147, 27148, 27149, 27150, 27151, 27152, 27153, 27154, 27155 were selected from the ORFs having the nucleotide sequence represented by SEQ ID NO: 1918.

The probes having the nucleotide sequence represented by SEQ ID NOS: 27156, 27157, 27158, 27159, 27160, 27161, 27162, 27163, 27164, 27165, 27166 were selected from the ORFs having the nucleotide sequence represented by SEQ ID NO: 1919.

The probes having the nucleotide sequence represented by SEQ ID NOS: 27167, 27168, 27169, 27170, 27171, 27172, 27173, 27174, 27175, 27176, 27177 were selected from the ORFs having the nucleotide sequence represented by SEQ ID NO: 1920.

The probes having the nucleotide sequence represented by SEQ ID NOS: 27178, 27179, 27180, 27181, 27182, 27183, 27184, 27185, 27186, 27187, 27188 were selected from the ORFs having the nucleotide sequence represented by SEQ ID NO: 1921.

The probes having the nucleotide sequence represented by SEQ ID NOS: 27189, 27190, 27191, 27192, 27193, 27194, 27195, 27196, 27197, 27198, 27199 were selected from the ORFs having the nucleotide sequence represented by SEQ ID NO: 1922.

The probes having the nucleotide sequence represented by SEQ ID NOS: 27200, 27201, 27202, 27203, 27204, 27205, 27206, 27207, 27208, 27209, 27210 were selected from the ORFs having the nucleotide sequence represented by SEQ ID NO: 1923.

The probes having the nucleotide sequence represented by SEQ ID NOS: 27211, 27212, 27213, 27214, 27215, 27216, 27217, 27218, 27219, 27220, 27221 were selected from the ORFs having the nucleotide sequence represented by SEQ ID NO: 1924.

The probes having the nucleotide sequence represented by SEQ ID NOS: 27222, 27223, 27224, 27225, 27226, 27227, 27228, 27229, 27230, 27231, 27232 were selected from the ORFs having the nucleotide sequence represented by SEQ ID NO: 1925.

The probes having the nucleotide sequence represented by SEQ ID NOS: 27233, 27234, 27235, 27236, 27237, 27238, 27239, 27240, 27241, 27242, 27243 were selected from the ORFs having the nucleotide sequence represented by SEQ ID NO: 1926.

The probes having the nucleotide sequence represented by SEQ ID NOS: 27244, 27245, 27246, 27247, 27248, 27249, 27250, 27251, 27252, 27253, 27254 were selected from the ORFs having the nucleotide sequence represented by SEQ ID NO: 1927.

The probes having the nucleotide sequence represented by SEQ ID NOS: 27255, 27256, 27257, 27258, 27259, 27260, 27261, 27262, 27263, 27264, 27265 were selected from the ORFs having the nucleotide sequence represented by SEQ ID NO: 1928.

The probes having the nucleotide sequence represented by SEQ ID NOS: 27266, 27267, 27268, 27269, 27270, 27271, 27272, 27273, 27274, 27275, 27276 were selected from the ORFs having the nucleotide sequence represented by SEQ ID NO: 1929.

The probes having the nucleotide sequence represented by SEQ ID NOS: 27277, 27278, 27279, 27280, 27281, 27282, 27283, 27284, 27285, 27286, 27287 were selected from the ORFs having the nucleotide sequence represented by SEQ ID NO: 1930.

The probes having the nucleotide sequence represented by SEQ ID NOS: 27288, 27289, 27290, 27291, 27292, 27293, 27294, 27295, 27296, 27297, 27298 were selected from the ORFs having the nucleotide sequence represented by SEQ ID NO: 1931.

The probes having the nucleotide sequence represented by SEQ ID NOS: 27299, 27300, 27301, 27302, 27303, 27304, 27305, 27306, 27307, 27308, 27309 were selected from the ORFs having the nucleotide sequence represented by SEQ ID NO: 1932.

The probes having the nucleotide sequence represented by SEQ ID NOS: 27310, 27311, 27312, 27313, 27314, 27315, 27316, 27317, 27318, 27319, 27320 were selected from the ORFs having the nucleotide sequence represented by SEQ ID NO: 1933.

The probes having the nucleotide sequence represented by SEQ ID NOS: 27321, 27322, 27323, 27324, 27325, 27326, 27327, 27328, 27329, 27330, 27331 were selected from the ORFs having the nucleotide sequence represented by SEQ ID NO: 1934.

The probes having the nucleotide sequence represented by SEQ ID NOS: 27332, 27333, 27334, 27335, 27336, 27337, 27338, 27339, 27340, 27341, 27342 were selected from the ORFs having the nucleotide sequence represented by SEQ ID NO: 1935.

The probes having the nucleotide sequence represented by SEQ ID NOS: 27343, 27344, 27345, 27346, 27347, 27348, 27349, 27350, 27351, 27352, 27353 were selected from the ORFs having the nucleotide sequence represented by SEQ ID NO: 1936.

The probes having the nucleotide sequence represented by SEQ ID NOS: 27354, 27355, 27356, 27357, 27358, 27359, 27360, 27361, 27362, 27363, 27364 were selected from the ORFs having the nucleotide sequence represented by SEQ ID NO: 1937.

The probes having the nucleotide sequence represented by SEQ ID NOS: 27365, 27366, 27367, 27368, 27369, 27370, 27371, 27372, 27373, 27374, 27375 were selected from the ORFs having the nucleotide sequence represented by SEQ ID NO: 1938.

The probes having the nucleotide sequence represented by SEQ ID NOS: 27376, 27377, 27378, 27379, 27380, 27381, 27382, 27383, 27384, 27385, 27386 were selected from the ORFs having the nucleotide sequence represented by SEQ ID NO: 1939.

The probes having the nucleotide sequence represented by SEQ ID NOS: 27387, 27388, 27389, 27390, 27391, 27392, 27393, 27394, 27395, 27396, 27397 were selected from the ORFs having the nucleotide sequence represented by SEQ ID NO: 1940.

The probes having the nucleotide sequence represented by SEQ ID NOS: 27398, 27399, 27400, 27401, 27402, 27403, 27404, 27405, 27406, 27407, 27408 were selected from the ORFs having the nucleotide sequence represented by SEQ ID NO: 1941.

The probes having the nucleotide sequence represented by SEQ ID NOS: 27409, 27410, 27411, 27412, 27413, 27414, 27415, 27416, 27417, 27418, 27419 were selected from the ORFs having the nucleotide sequence represented by SEQ ID NO: 1942.

The probes having the nucleotide sequence represented by SEQ ID NOS: 27420, 27421, 27422, 27423, 27424, 27425, 27426, 27427, 27428, 27429, 27430 were selected from the ORFs having the nucleotide sequence represented by SEQ ID NO: 1943.

The probes having the nucleotide sequence represented by SEQ ID NOS: 27431, 27432, 27433, 27434, 27435, 27436, 27437, 27438, 27439, 27440, 27441 were selected from the ORFs having the nucleotide sequence represented by SEQ ID NO: 1944.

The probes having the nucleotide sequence represented by SEQ ID NOS: 27442, 27443, 27444, 27445, 27446, 27447, 27448, 27449, 27450, 27451, 27452 were selected from the ORFs having the nucleotide sequence represented by SEQ ID NO: 1945.

The probes having the nucleotide sequence represented by SEQ ID NOS: 27453, 27454, 27455, 27456, 27457, 27458, 27459, 27460, 27461, 27462, 27463 were selected from the ORFs having the nucleotide sequence represented by SEQ ID NO: 1946.

The probes having the nucleotide sequence represented by SEQ ID NOS: 27464, 27465, 27466, 27467, 27468, 27469, 27470, 27471, 27472, 27473, 27474 were selected from the ORFs having the nucleotide sequence represented by SEQ ID NO: 1947.

The probes having the nucleotide sequence represented by SEQ ID NOS: 27475, 27476, 27477, 27478, 27479, 27480, 27481, 27482, 27483, 27484, 27485 were selected from the ORFs having the nucleotide sequence represented by SEQ ID NO: 1948.

The probes having the nucleotide sequence represented by SEQ ID NOS: 27486, 27487, 27488, 27489, 27490, 27491, 27492, 27493, 27494, 27495, 27496 were selected from the ORFs having the nucleotide sequence represented by SEQ ID NO: 1949.

The probes having the nucleotide sequence represented by SEQ ID NOS: 27497, 27498, 27499, 27500, 27501, 27502, 27503, 27504, 27505, 27506, 27507 were selected from the ORFs having the nucleotide sequence represented by SEQ ID NO: 1950.

The probes having the nucleotide sequence represented by SEQ ID NOS: 27508, 27509, 27510, 27511, 27512, 27513, 27514, 27515, 27516, 27517, 27518 were selected from the ORFs having the nucleotide sequence represented by SEQ ID NO: 1951.

The probes having the nucleotide sequence represented by SEQ ID NOS: 27519, 27520, 27521, 27522, 27523, 27524, 27525, 27526, 27527, 27528, 27529 were selected from the ORFs having the nucleotide sequence represented by SEQ ID NO: 1952.

The probes having the nucleotide sequence represented by SEQ ID NOS: 27530, 27531, 27532, 27533, 27534, 27535, 27536, 27537, 27538, 27539, 27540 were selected from the ORFs having the nucleotide sequence represented by SEQ ID NO: 1953.

The probes having the nucleotide sequence represented by SEQ ID NOS: 27541, 27542, 27543, 27544, 27545, 27546, 27547, 27548, 27549, 27550, 27551 were selected from the ORFs having the nucleotide sequence represented by SEQ ID NO: 1954.

The probes having the nucleotide sequence represented by SEQ ID NOS: 27552, 27553, 27554, 27555, 27556, 27557, 27558, 27559, 27560, 27561, 27562 were selected from the ORFs having the nucleotide sequence represented by SEQ ID NO: 1955.

The probes having the nucleotide sequence represented by SEQ ID NOS: 27563, 27564, 27565, 27566, 27567, 27568, 27569, 27570, 27571, 27572, 27573 were selected from the ORFs having the nucleotide sequence represented by SEQ ID NO: 1956.

The probes having the nucleotide sequence represented by SEQ ID NOS: 27574, 27575, 27576, 27577, 27578, 27579, 27580, 27581, 27582, 27583, 27584 were selected from the ORFs having the nucleotide sequence represented by SEQ ID NO: 1957.

The probes having the nucleotide sequence represented by SEQ ID NOS: 27585, 27586, 27587, 27588, 27589, 27590, 27591, 27592, 27593, 27594, 27595 were selected from the ORFs having the nucleotide sequence represented by SEQ ID NO: 1958.

The probes having the nucleotide sequence represented by SEQ ID NOS: 27596, 27597, 27598, 27599, 27600, 27601, 27602, 27603, 27604, 27605, 27606 were selected from the ORFs having the nucleotide sequence represented by SEQ ID NO: 1959.

The probes having the nucleotide sequence represented by SEQ ID NOS: 27607, 27608, 27609, 27610, 27611, 27612, 27613, 27614, 27615, 27616, 27617 were selected from the ORFs having the nucleotide sequence represented by SEQ ID NO: 1960.

The probes having the nucleotide sequence represented by SEQ ID NOS: 27618, 27619, 27620, 27621, 27622, 27623, 27624, 27625, 27626, 27627, 27628 were selected from the ORFs having the nucleotide sequence represented by SEQ ID NO: 1961.

The probes having the nucleotide sequence represented by SEQ ID NOS: 27629, 27630, 27631, 27632, 27633, 27634, 27635, 27636, 27637, 27638, 27639 were selected from the ORFs having the nucleotide sequence represented by SEQ ID NO: 1962.

The probes having the nucleotide sequence represented by SEQ ID NOS: 27640, 27641, 27642, 27643, 27644, 27645, 27646, 27647, 27648, 27649, 27650 were selected from the ORFs having the nucleotide sequence represented by SEQ ID NO: 1963.

The probes having the nucleotide sequence represented by SEQ ID NOS: 27651, 27652, 27653, 27654, 27655, 27656, 27657, 27658, 27659, 27660, 27661 were selected from the ORFs having the nucleotide sequence represented by SEQ ID NO: 1964.

The probes having the nucleotide sequence represented by SEQ ID NOS: 27662, 27663, 27664, 27665, 27666, 27667, 27668, 27669, 27670, 27671, 27672 were selected from the ORFs having the nucleotide sequence represented by SEQ ID NO: 1965.

The probes having the nucleotide sequence represented by SEQ ID NOS: 27673, 27674, 27675, 27676, 27677, 27678, 27679, 27680, 27681, 27682, 27683 were selected from the ORFs having the nucleotide sequence represented by SEQ ID NO: 1966.

The probes having the nucleotide sequence represented by SEQ ID NOS: 27684, 27685, 27686, 27687, 27688, 27689, 27690, 27691, 27692, 27693, 27694 were selected from the ORFs having the nucleotide sequence represented by SEQ ID NO: 1967.

The probes having the nucleotide sequence represented by SEQ ID NOS: 27695, 27696, 27697, 27698, 27699, 27700, 27701, 27702, 27703, 27704, 27705 were selected from the ORFs having the nucleotide sequence represented by SEQ ID NO: 1968.

The probes having the nucleotide sequence represented by SEQ ID NOS: 27706, 27707, 27708, 27709, 27710, 27711, 27712, 27713, 27714, 27715, 27716 were selected from the ORFs having the nucleotide sequence represented by SEQ ID NO: 1969.

The probes having the nucleotide sequence represented by SEQ ID NOS: 27717, 27718, 27719, 27720, 27721, 27722, 27723, 27724, 27725, 27726, 27727 were selected from the ORFs having the nucleotide sequence represented by SEQ ID NO: 1970.

The probes having the nucleotide sequence represented by SEQ ID NOS: 27728, 27729, 27730, 27731, 27732, 27733, 27734, 27735, 27736, 27737, 27738 were selected from the ORFs having the nucleotide sequence represented by SEQ ID NO: 1971.

The probes having the nucleotide sequence represented by SEQ ID NOS: 27739, 27740, 27741, 27742, 27743, 27744, 27745, 27746, 27747, 27748, 27749 were selected from the ORFs having the nucleotide sequence represented by SEQ ID NO: 1972.

The probes having the nucleotide sequence represented by SEQ ID NOS: 27750, 27751, 27752, 27753, 27754, 27755, 27756, 27757, 27758, 27759, 27760 were selected from the ORFs having the nucleotide sequence represented by SEQ ID NO: 1973.

The probes having the nucleotide sequence represented by SEQ ID NOS: 27761, 27762, 27763, 27764, 27765, 27766, 27767, 27768, 27769, 27770, 27771 were selected from the ORFs having the nucleotide sequence represented by SEQ ID NO: 1974.

The probes having the nucleotide sequence represented by SEQ ID NOS: 27772, 27773, 27774, 27775, 27776, 27777, 27778, 27779, 27780, 27781, 27782 were selected from the ORFs having the nucleotide sequence represented by SEQ ID NO: 1975.

The probes having the nucleotide sequence represented by SEQ ID NOS: 27783, 27784, 27785, 27786, 27787, 27788, 27789, 27790, 27791, 27792, 27793 were selected from the ORFs having the nucleotide sequence represented by SEQ ID NO: 1976.

The probes having the nucleotide sequence represented by SEQ ID NOS: 27794, 27795, 27796, 27797, 27798, 27799, 27800, 27801, 27802, 27803, 27804 were selected from the ORFs having the nucleotide sequence represented by SEQ ID NO: 1977.

The probes having the nucleotide sequence represented by SEQ ID NOS: 27805, 27806, 27807, 27808, 27809, 27810, 27811, 27812, 27813, 27814, 27815 were selected from the ORFs having the nucleotide sequence represented by SEQ ID NO: 1978.

The probes having the nucleotide sequence represented by SEQ ID NOS: 27816, 27817, 27818, 27819, 27820, 27821, 27822, 27823, 27824, 27825, 27826 were selected from the ORFs having the nucleotide sequence represented by SEQ ID NO: 1979.

The probes having the nucleotide sequence represented by SEQ ID NOS: 27827, 2782.8, 27829, 27830, 27831, 27832, 27833, 27834, 27835, 27836, 27837 were selected from the ORFs having the nucleotide sequence represented by SEQ ID NO: 1980.

The probes having the nucleotide sequence represented by SEQ ID NOS: 27838, 27839, 27840, 27841, 27842, 27843, 27844, 27845, 27846, 27847, 27848 were selected from the ORFs having the nucleotide sequence represented by SEQ ID NO: 1981.

The probes having the nucleotide sequence represented by SEQ ID NOS: 27849, 27850, 27851, 27852, 27853, 27854, 27855, 27856, 27857, 27858, 27859 were selected from the ORFs having the nucleotide sequence represented by SEQ ID NO: 1982.

The probes having the nucleotide sequence represented by SEQ ID NOS: 27860, 27861, 27862, 27863, 27864, 27865, 27866, 27867, 27868, 27869, 27870 were selected from the ORFs having the nucleotide sequence represented by SEQ ID NO: 1983.

The probes having the nucleotide sequence represented by SEQ ID NOS: 27871, 27872, 27873, 27874, 27875, 27876, 27877, 27878, 27879, 27880, 27881 were selected from the ORFs having the nucleotide sequence represented by SEQ ID NO: 1984.

The probes having the nucleotide sequence represented by SEQ ID NOS: 27882, 27883, 27884, 27885, 27886, 27887, 27888, 27889, 27890, 27891, 27892 were selected from the ORFs having the nucleotide sequence represented by SEQ ID NO: 1985.

The probes having the nucleotide sequence represented by SEQ ID NOS: 27893, 27894, 27895, 27896, 27897, 27898, 27899, 27900, 27901, 27902, 27903 were selected from the ORFs having the nucleotide sequence represented by SEQ ID NO: 1986.

The probes having the nucleotide sequence represented by SEQ ID NOS: 27904, 27905, 27906, 27907, 27908, 27909, 27910, 27911, 27912, 27913, 27914 were selected from the ORFs having the nucleotide sequence represented by SEQ ID NO: 1987.

The probes having the nucleotide sequence represented by SEQ ID NOS: 27915, 27916, 27917, 27918, 27919, 27920, 27921, 27922, 27923, 27924, 27925 were selected from the ORFs having the nucleotide sequence represented by SEQ ID NO: 1988.

The probes having the nucleotide sequence represented by SEQ ID NOS: 27926, 27927, 27928, 27929, 27930, 27931, 27932, 27933, 27934, 27935, 27936 were selected from the ORFs having the nucleotide sequence represented by SEQ ID NO: 1989.

The probes having the nucleotide sequence represented by SEQ ID NOS: 27937, 27938, 27939, 27940, 27941, 27942, 27943, 27944, 27945, 27946, 27947 were selected from the ORFs having the nucleotide sequence represented by SEQ ID NO: 1990.

The probes having the nucleotide sequence represented by SEQ ID NOS: 27948, 27949, 27950, 27951, 27952, 27953, 27954, 27955, 27956, 27957, 27958 were selected from the ORFs having the nucleotide sequence represented by SEQ ID NO: 1991.

The probes having the nucleotide sequence represented by SEQ ID NOS: 27959, 27960, 27961, 27962, 27963, 27964, 27965, 27966, 27967, 27968, 27969 were selected from the ORFs having the nucleotide sequence represented by SEQ ID NO: 1992.

The probes having the nucleotide sequence represented by SEQ ID NOS: 27970, 27971, 27972, 27973, 27974, 27975, 27976, 27977, 27978, 27979, 27980 were selected from the ORFs having the nucleotide sequence represented by SEQ ID NO: 1993.

The probes having the nucleotide sequence represented by SEQ ID NOS: 27981, 27982, 27983, 27984, 27985, 27986, 27987, 27988, 27989, 27990 were selected from the ORFs having the nucleotide sequence represented by SEQ ID NO: 1994.

The probes having the nucleotide sequence represented by SEQ ID NOS: 27991, 27992, 27993, 27994, 27995, 27996, 27997, 27998, 27999, 28000, 28001 were selected from the ORFs having the nucleotide sequence represented by SEQ ID NO: 1995.

The probes having the nucleotide sequence represented by SEQ ID NOS: 28002, 28003, 28004, 28005, 28006, 28007, 28008, 28009, 28010, 28011, 28012 were selected from the ORFs having the nucleotide sequence represented by SEQ ID NO: 1996.

The probes having the nucleotide sequence represented by SEQ ID NOS: 28013, 28014, 28015, 28016, 28017, 28018, 28019, 28020, 28021, 28022, 28023 were selected from the ORFs having the nucleotide sequence represented by SEQ ID NO: 1997.

The probes having the nucleotide sequence represented by SEQ ID NOS: 28024, 28025, 28026, 28027, 28028, 28029, 28030, 28031, 28032, 28033, 28034 were selected from the ORFs having the nucleotide sequence represented by SEQ ID NO: 1998.

The probes having the nucleotide sequence represented by SEQ ID NOS: 28035, 28036, 28037, 28038, 28039, 28040, 28041, 28042, 28043, 28044, 28045 were selected from the ORFs having the nucleotide sequence represented by SEQ ID NO: 1999.

The probes having the nucleotide sequence represented by SEQ ID NOS: 28046, 28047, 28048, 28049, 28050, 28051, 28052, 28053, 28054, 28055, 28056 were selected from the ORFs having the nucleotide sequence represented by SEQ ID NO: 2000.

The probes having the nucleotide sequence represented by SEQ ID NOS: 28057, 28058, 28059, 28060, 28061, 28062, 28063, 28064, 28065, 28066, 28067 were selected from the ORFs having the nucleotide sequence represented by SEQ ID NO: 2001.

The probes having the nucleotide sequence represented by SEQ ID NOS: 28068, 28069, 28070, 28071, 28072, 28073, 28074, 28075, 28076, 28077, 28078 were selected from the ORFs having the nucleotide sequence represented by SEQ ID NO: 2002.

The probes having the nucleotide sequence represented by SEQ ID NOS: 28079, 28080, 28081, 28082, 28083, 28084, 28085, 28086, 28087, 28088, 28089 were selected from the ORFs having the nucleotide sequence represented by SEQ ID NO: 2003.

The probes having the nucleotide sequence represented by SEQ ID NOS: 28090, 28091, 28092, 28093, 28094, 28095, 28096, 28097, 28098, 28099, 28100 were selected from the ORFs having the nucleotide sequence represented by SEQ ID NO: 2004.

The probes having the nucleotide sequence represented by SEQ ID NOS: 28101, 28102, 28103, 28104, 28105, 28106, 28107, 28108, 28109, 28110, 28111 were selected from the ORFs having the nucleotide sequence represented by SEQ ID NO: 2005.

The probes having the nucleotide sequence represented by SEQ ID NOS: 28112, 28113, 28114, 28115, 28116, 28117, 28118, 28119, 28120, 28121, 28122 were selected from the ORFs having the nucleotide sequence represented by SEQ ID NO: 2006.

The probes having the nucleotide sequence represented by SEQ ID NOS: 28123, 28124, 28125, 28126, 28127, 28128, 28129, 28130, 28131, 28132, 28133 were selected from the ORFs having the nucleotide sequence represented by SEQ ID NO: 2007.

The probes having the nucleotide sequence represented by SEQ ID NOS: 28134, 28135, 28136, 28137, 28138, 28139, 28140, 28141, 28142, 28143, 28144 were selected from the ORFs having the nucleotide sequence represented by SEQ ID NO: 2008.

The probes having the nucleotide sequence represented by SEQ ID NOS: 28145, 28146, 28147, 28148, 28149, 28150, 28151, 28152, 28153, 28154, 28155 were selected from the ORFs having the nucleotide sequence represented by SEQ ID NO: 2009.

The probes having the nucleotide sequence represented by SEQ ID NOS: 28156, 28157, 28158, 28159, 28160, 28161, 28162, 28163, 28164, 28165, 28166 were selected from the ORFs having the nucleotide sequence represented by SEQ ID NO: 2010.

The probes having the nucleotide sequence represented by SEQ ID NOS: 28167, 28168, 28169, 28170, 28171, 28172, 28173, 28174, 28175, 28176, 28177 were selected from the ORFs having the nucleotide sequence represented by SEQ ID NO: 2011.

The probes having the nucleotide sequence represented by SEQ ID NOS: 28178, 28179, 28180, 28181, 28182, 28183, 28184, 28185, 28186, 28187, 28188 were selected from the ORFs having the nucleotide sequence represented by SEQ ID NO: 2012.

The probes having the nucleotide sequence represented by SEQ ID NOS: 28189, 28190, 28191, 28192, 28193, 28194, 28195, 28196, 28197, 28198, 28199 were selected from the ORFs having the nucleotide sequence represented by SEQ ID NO: 2013.

The probes having the nucleotide sequence represented by SEQ ID NOS: 28200, 28201, 28202, 28203, 28204, 28205, 28206, 28207, 28208, 28209, 28210 were selected from the ORFs having the nucleotide sequence represented by SEQ ID NO: 2014.

The probes having the nucleotide sequence represented by SEQ ID NOS: 28211, 28212, 28213, 28214, 28215, 28216, 28217, 28218, 28219, 28220, 28221 were selected from the ORFs having the nucleotide sequence represented by SEQ ID NO: 2015.

The probes having the nucleotide sequence represented by SEQ ID NOS: 28222, 28223, 28224, 28225, 28226, 28227, 28228, 28229, 28230, 28231, 28232 were selected from the ORFs having the nucleotide sequence represented by SEQ ID NO: 2016.

The probes having the nucleotide sequence represented by SEQ ID NOS: 28233, 28234, 28235, 28236, 28237, 28238, 28239, 28240, 28241, 28242, 28243 were selected from the ORFs having the nucleotide sequence represented by SEQ ID NO: 2017.

The probes having the nucleotide sequence represented by SEQ ID NOS: 28244, 28245, 28246, 28247, 28248, 28249, 28250, 28251, 28252, 28253, 28254 were selected from the ORFs having the nucleotide sequence represented by SEQ ID NO: 2018.

The probes having the nucleotide sequence represented by SEQ ID NOS: 28255, 28256, 28257, 28258, 28259, 28260, 28261, 28262, 28263, 28264, 28265 were selected from the ORFs having the nucleotide sequence represented by SEQ ID NO: 2019.

The probes having the nucleotide sequence represented by SEQ ID NOS: 28266, 28267, 28268, 28269, 28270, 28271, 28272, 28273, 28274, 28275, 28276 were selected from the ORFs having the nucleotide sequence represented by SEQ ID NO: 2020.

The probes having the nucleotide sequence represented by SEQ ID NOS: 28277, 28278, 28279, 28280, 28281, 28282, 28283, 28284, 28285, 28286, 28287 were selected from the ORFs having the nucleotide sequence represented by SEQ ID NO: 2021.

The probes having the nucleotide sequence represented by SEQ ID NOS: 28288, 28289, 28290, 28291, 28292, 28293, 28294, 28295, 28296, 28297, 28298 were selected from the ORFs having the nucleotide sequence represented by SEQ ID NO: 2022.

The probes having the nucleotide sequence represented by SEQ ID NOS: 28299, 28300, 28301, 28302, 28303, 28304, 28305, 28306, 28307, 28308, 28309 were selected from the ORFs having the nucleotide sequence represented by SEQ ID NO: 2023.

The probes having the nucleotide sequence represented by SEQ ID NOS: 28310, 28311, 28312, 28313, 28314, 28315, 28316, 28317, 28318, 28319, 28320 were selected from the ORFs having the nucleotide sequence represented by SEQ ID NO: 2024.

The probes having the nucleotide sequence represented by SEQ ID NOS: 28321, 28322, 28323, 28324, 28325, 28326, 28327, 28328, 28329, 28330, 28331 were selected from the ORFs having the nucleotide sequence represented by SEQ ID NO: 2025.

The probes having the nucleotide sequence represented by SEQ ID NOS: 28332, 28333, 28334, 28335, 28336, 28337, 28338, 28339, 28340, 28341, 28342 were selected from the ORFs having the nucleotide sequence represented by SEQ ID NO: 2026.

The probes having the nucleotide sequence represented by SEQ ID NOS: 28343, 28344, 28345, 28346, 28347, 28348, 28349, 28350, 28351, 28352, 28353 were selected from the ORFs having the nucleotide sequence represented by SEQ ID NO: 2027.

The probes having the nucleotide sequence represented by SEQ ID NOS: 28354, 28355, 28356, 28357, 28358, 28359, 28360, 28361, 28362, 28363, 28364 were selected from the ORFs having the nucleotide sequence represented by SEQ ID NO: 2028.

The probes having the nucleotide sequence represented by SEQ ID NOS: 28365, 28366, 28367, 28368, 28369, 28370, 28371, 28372, 28373, 28374, 28375 were selected from the ORFs having the nucleotide sequence represented by SEQ ID NO: 2029.

The probes having the nucleotide sequence represented by SEQ ID NOS: 28376, 28377, 28378, 28379, 28380, 28381, 28382, 28383, 28384, 28385, 28386 were selected from the ORFs having the nucleotide sequence represented by SEQ ID NO: 2030.

The probes having the nucleotide sequence represented by SEQ ID NOS: 28387, 28388, 28389, 28390, 28391, 28392, 28393, 28394, 28395, 28396, 28397 were selected from the ORFs having the nucleotide sequence represented by SEQ ID NO: 2031.

The probes having the nucleotide sequence represented by SEQ ID NOS: 28398, 28399, 28400, 28401, 28402, 28403, 28404, 28405, 28406, 28407, 28408 were selected from the ORFs having the nucleotide sequence represented by SEQ ID NO: 2032.

The probes having the nucleotide sequence represented by SEQ ID NOS: 28409, 28410, 28411, 28412, 28413, 28414, 28415, 28416, 28417, 28418, 28419 were selected from the ORFs having the nucleotide sequence represented by SEQ ID NO: 2033.

The probes having the nucleotide sequence represented by SEQ ID NOS: 28420, 28421, 28422, 28423, 28424, 28425, 28426, 28427, 28428, 28429, 28430 were selected from the ORFs having the nucleotide sequence represented by SEQ ID NO: 2034.

The probes having the nucleotide sequence represented by SEQ ID NOS: 28431, 28432, 28433, 28434, 28435, 28436, 28437, 28438, 28439, 28440, 28441 were selected from the ORFs having the nucleotide sequence represented by SEQ ID NO: 2035.

The probes having the nucleotide sequence represented by SEQ ID NOS: 28442, 28443, 28444, 28445, 28446, 28447, 28448, 28449, 28450, 28451, 28452 were selected from the ORFs having the nucleotide sequence represented by SEQ ID NO: 2036.

The probes having the nucleotide sequence represented by SEQ ID NOS: 28453, 28454, 28455, 28456, 28457, 28458, 28459, 28460, 28461, 28462, 28463 were selected from the ORFs having the nucleotide sequence represented by SEQ ID NO: 2037.

The probes having the nucleotide sequence represented by SEQ ID NOS: 28464, 28465, 28466, 28467, 28468, 28469, 28470, 28471, 28472, 28473, 28474 were selected from the ORFs having the nucleotide sequence represented by SEQ ID NO: 2038.

The probes having the nucleotide sequence represented by SEQ ID NOS: 28475, 28476, 28477, 28478, 28479, 28480, 28481, 28482, 28483, 28484, 28485 were selected from the ORFs having the nucleotide sequence represented by SEQ ID NO: 2039.

The probes having the nucleotide sequence represented by SEQ ID NOS: 28486, 28487, 28488, 28489, 28490, 28491, 28492, 28493, 28494, 28495, 28496 were selected from the ORFs having the nucleotide sequence represented by SEQ ID NO: 2040.

The probes having the nucleotide sequence represented by SEQ ID NOS: 28497, 28498, 28499, 28500, 28501, 28502, 28503, 28504, 28505, 28506, 28507 were selected from the ORFs having the nucleotide sequence represented by SEQ ID NO: 2041.

The probes having the nucleotide sequence represented by SEQ ID NOS: 28508, 28509, 28510, 28511, 28512, 28513, 28514, 28515, 28516, 28517, 28518 were selected from the ORFs having the nucleotide sequence represented by SEQ ID NO: 2042.

The probes having the nucleotide sequence represented by SEQ ID NOS: 28519, 28520, 28521, 28522, 28523, 28524, 28525, 28526, 28527, 28528, 28529 were selected from the ORFs having the nucleotide sequence represented by SEQ ID NO: 2043.

The probes having the nucleotide sequence represented by SEQ ID NOS: 28530, 28531, 28532, 28533, 28534, 28535, 28536, 28537, 28538, 28539, 28540 were selected from the ORFs having the nucleotide sequence represented by SEQ ID NO: 2044.

The probes having the nucleotide sequence represented by SEQ ID NOS: 28541, 28542, 28543, 28544, 28545, 28546, 28547, 28548, 28549, 28550, 28551 were selected from the ORFs having the nucleotide sequence represented by SEQ ID NO: 2045.

The probes having the nucleotide sequence represented by SEQ ID NOS: 28552, 28553, 28554, 28555, 28556, 28557, 28558, 28559, 28560, 28561, 28562 were selected from the ORFs having the nucleotide sequence represented by SEQ ID NO: 2046.

The probes having the nucleotide sequence represented by SEQ ID NOS: 28563, 28564, 28565, 28566, 28567, 28568, 28569, 28570, 28571, 28572, 28573 were selected from the ORFs having the nucleotide sequence represented by SEQ ID NO: 2047.

The probes having the nucleotide sequence represented by SEQ ID NOS: 28574, 28575, 28576, 28577, 28578, 28579, 28580, 28581, 28582, 28583, 28584 were selected from the ORFs having the nucleotide sequence represented by SEQ ID NO: 2048.

The probes having the nucleotide sequence represented by SEQ ID NOS: 28585, 28586, 28587, 28588, 28589, 28590, 28591, 28592, 28593, 28594, 28595 were selected from the ORFs having the nucleotide sequence represented by SEQ ID NO: 2049.

The probes having the nucleotide sequence represented by SEQ ID NOS: 28596, 28597, 28598, 28599, 28600, 28601, 28602, 28603, 28604, 28605, 28606 were selected from the ORFs having the nucleotide sequence represented by SEQ ID NO: 2050.

The probes having the nucleotide sequence represented by SEQ ID NOS: 28607, 28608, 28609, 28610, 28611, 28612, 28613, 28614, 28615, 28616, 28617 were selected from the ORFs having the nucleotide sequence represented by SEQ ID NO: 2051.

The probes having the nucleotide sequence represented by SEQ ID NOS: 28618, 28619, 28620, 28621, 28622, 28623, 28624, 28625, 28626, 28627, 28628 were selected from the ORFs having the nucleotide sequence represented by SEQ ID NO: 2052.

The probes having the nucleotide sequence represented by SEQ ID NOS: 28629, 28630, 28631, 28632, 28633, 28634, 28635, 28636, 28637, 28638, 28639 were selected from the ORFs having the nucleotide sequence represented by SEQ ID NO: 2053.

The probes having the nucleotide sequence represented by SEQ ID NOS: 28640, 28641, 28642, 28643, 28644, 28645, 28646, 28647, 28648, 28649, 28650 were selected from the ORFs having the nucleotide sequence represented by SEQ ID NO: 2054.

The probes having the nucleotide sequence represented by SEQ ID NOS: 28651, 28652, 28653, 28654, 28655, 28656, 28657, 28658, 28659, 28660, 28661 were selected from the ORFs having the nucleotide sequence represented by SEQ ID NO: 2055.

The probes having the nucleotide sequence represented by SEQ ID NOS: 28662, 28663, 28664, 28665, 28666, 28667, 28668, 28669, 28670, 28671, 28672 were selected from the ORFs having the nucleotide sequence represented by SEQ ID NO: 2056.

The probes having the nucleotide sequence represented by SEQ ID NOS: 28673, 28674, 28675, 28676, 28677, 28678, 28679, 28680, 28681, 28682, 28683 were selected from the ORFs having the nucleotide sequence represented by SEQ ID NO: 2057.

The probes having the nucleotide sequence represented by SEQ ID NOS: 28684, 28685, 28686, 28687, 28688, 28689, 28690, 28691, 28692, 28693, 28694 were selected from the ORFs having the nucleotide sequence represented by SEQ ID NO: 2058.

The probes having the nucleotide sequence represented by SEQ ID NOS: 28695, 28696, 28697, 28698, 28699, 28700, 28701, 28702, 28703, 28704, 28705 were selected from the ORFs having the nucleotide sequence represented by SEQ ID NO: 2059.

The probes having the nucleotide sequence represented by SEQ ID NOS: 28706, 28707, 28708, 28709, 28710, 28711, 28712, 28713, 28714, 28715, 28716 were selected from the ORFs having the nucleotide sequence represented by SEQ ID NO: 2060.

The probes having the nucleotide sequence represented by SEQ ID NOS: 28717, 28718, 28719, 28720, 28721, 28722, 28723, 28724, 28725, 28726, 28727 were selected from the ORFs having the nucleotide sequence represented by SEQ ID NO: 2061.

The probes having the nucleotide sequence represented by SEQ ID NOS: 28728, 28729, 28730, 28731, 28732, 28733, 28734, 28735, 28736, 28737, 28738 were selected from the ORFs having the nucleotide sequence represented by SEQ ID NO: 2062.

The probes having the nucleotide sequence represented by SEQ ID NOS: 28739, 28740, 28741, 28742, 28743, 28744, 28745, 28746, 28747, 28748, 28749 were selected from the ORFs having the nucleotide sequence represented by SEQ ID NO: 2063.

The probes having the nucleotide sequence represented by SEQ ID NOS: 28750, 28751, 28752, 28753, 28754, 28755, 28756, 28757, 28758, 28759, 28760 were selected from the ORFs having the nucleotide sequence represented by SEQ ID NO: 2064.

The probes having the nucleotide sequence represented by SEQ ID NOS: 28761, 28762, 28763, 28764, 28765, 28766, 28767, 28768, 28769, 28770, 28771 were selected from the ORFs having the nucleotide sequence represented by SEQ ID NO: 2065.

The probes having the nucleotide sequence represented by SEQ ID NOS: 28772, 28773, 28774, 28775, 28776, 28777, 28778, 28779, 28780, 28781, 28782 were selected from the ORFs having the nucleotide sequence represented by SEQ ID NO: 2066.

The probes having the nucleotide sequence represented by SEQ ID NOS: 28783, 28784, 28785, 28786, 28787, 28788, 28789, 28790, 28791, 28792, 28793 were selected from the ORFs having the nucleotide sequence represented by SEQ ID NO: 2067.

The probes having the nucleotide sequence represented by SEQ ID NOS: 28794, 28795, 28796, 28797, 28798, 28799, 28800, 28801, 28802, 28803, 28804 were selected from the ORFs having the nucleotide sequence represented by SEQ ID NO: 2068.

The probes having the nucleotide sequence represented by SEQ ID NOS: 28805, 28806, 28807, 28808, 28809, 28810, 28811, 28812, 28813, 28814, 28815 were selected from the ORFs having the nucleotide sequence represented by SEQ ID NO: 2069.

The probes having the nucleotide sequence represented by SEQ ID NOS: 28816, 28817, 28818, 28819, 28820, 28821, 28822, 28823, 28824, 28825, 28826 were selected from the ORFs having the nucleotide sequence represented by SEQ ID NO: 2070.

The probes having the nucleotide sequence represented by SEQ ID NOS: 28827, 28828, 28829, 28830, 28831, 28832, 28833, 28834, 28835, 28836, 28837 were selected from the ORFs having the nucleotide sequence represented by SEQ ID NO: 2071.

The probes having the nucleotide sequence represented by SEQ ID NOS: 28838, 28839, 28840, 28841, 28842, 28843, 28844, 28845, 28846, 28847, 28848 were selected from the ORFs having the nucleotide sequence represented by SEQ ID NO: 2072.

The probes having the nucleotide sequence represented by SEQ ID NOS: 28849, 28850, 28851, 28852, 28853, 28854, 28855, 28856, 28857, 28858, 28859 were selected from the ORFs having the nucleotide sequence represented by SEQ ID NO: 2073.

The probes having the nucleotide sequence represented by SEQ ID NOS: 28860, 28861, 28862, 28863, 28864, 28865, 28866, 28867, 28868, 28869, 28870 were selected from the ORFs having the nucleotide sequence represented by SEQ ID NO: 2074.

The probes having the nucleotide sequence represented by SEQ ID NOS: 28871, 28872, 28873, 28874, 28875, 28876, 28877, 28878, 28879, 28880, 28881 were selected from the ORFs having the nucleotide sequence represented by SEQ ID NO: 2075.

The probes having the nucleotide sequence represented by SEQ ID NOS: 28882, 28883, 28884, 28885, 28886, 28887, 28888, 28889, 28890, 28891, 28892 were selected from the ORFs having the nucleotide sequence represented by SEQ ID NO: 2076.

The probes having the nucleotide sequence represented by SEQ ID NOS: 28893, 28894, 28895, 28896, 28897, 28898, 28899, 28900, 28901, 28902, 28903 were selected from the ORFs having the nucleotide sequence represented by SEQ ID NO: 2077.

The probes having the nucleotide sequence represented by SEQ ID NOS: 28904, 28905, 28906, 28907, 28908, 28909, 28910, 28911, 28912, 28913, 28914 were selected from the ORFs having the nucleotide sequence represented by SEQ ID NO: 2078.

The probes having the nucleotide sequence represented by SEQ ID NOS: 28915, 28916, 28917, 28918, 28919, 28920, 28921, 28922, 28923, 28924, 28925 were selected from the ORFs having the nucleotide sequence represented by SEQ ID NO: 2079.

The probes having the nucleotide sequence represented by SEQ ID NOS: 28926, 28927, 28928, 28929, 28930, 28931, 28932, 28933, 28934, 28935, 28936 were selected from the ORFs having the nucleotide sequence represented by SEQ ID NO: 2080.

The probes having the nucleotide sequence represented by SEQ ID NOS: 28937, 28938, 28939, 28940, 28941, 28942, 28943, 28944, 28945, 28946, 28947 were selected from the ORFs having the nucleotide sequence represented by SEQ ID NO: 2081.

The probes having the nucleotide sequence represented by SEQ ID NOS: 28948, 28949, 28950, 28951, 28952, 28953, 28954, 28955, 28956, 28957, 28958 were selected from the ORFs having the nucleotide sequence represented by SEQ ID NO: 2082.

The probes having the nucleotide sequence represented by SEQ ID NOS: 28959, 28960, 28961, 28962, 28963, 28964, 28965, 28966, 28967, 28968, 28969 were selected from the ORFs having the nucleotide sequence represented by SEQ ID NO: 2083.

The probes having the nucleotide sequence represented by SEQ ID NOS: 28970, 28971, 28972, 28973, 28974, 28975, 28976, 28977, 28978, 28979, 28980 were selected from the ORFs having the nucleotide sequence represented by SEQ ID NO: 2084.

The probes having the nucleotide sequence represented by SEQ ID NOS: 28981, 28982, 28983, 28984, 28985, 28986, 28987, 28988, 28989, 28990, 28991 were selected from the ORFs having the nucleotide sequence represented by SEQ ID NO: 2085.

The probes having the nucleotide sequence represented by SEQ ID NOS: 28992, 28993, 28994, 28995, 28996, 28997, 28998, 28999, 29000, 29001, 29002 were selected from the ORFs having the nucleotide sequence represented by SEQ ID NO: 2086.

The probes having the nucleotide sequence represented by SEQ ID NOS: 29003, 29004, 29005, 29006, 29007, 29008, 29009, 29010, 29011, 29012, 29013 were selected from the ORFs having the nucleotide sequence represented by SEQ ID NO: 2087.

The probes having the nucleotide sequence represented by SEQ ID NOS: 29014, 29015, 29016, 29017, 29018, 29019, 29020, 29021, 29022, 29023, 29024 were selected from the ORFs having the nucleotide sequence represented by SEQ ID NO: 2088.

The probes having the nucleotide sequence represented by SEQ ID NOS: 29025, 29026, 29027, 29028, 29029, 29030, 29031, 29032, 29033, 29034, 29035 were selected from the ORFs having the nucleotide sequence represented by SEQ ID NO: 2089.

The probes having the nucleotide sequence represented by SEQ ID NOS: 29036, 29037, 29038, 29039, 29040, 29041, 29042, 29043, 29044, 29045, 29046 were selected from the ORFs having the nucleotide sequence represented by SEQ ID NO: 2090.

The probes having the nucleotide sequence represented by SEQ ID NOS: 29047, 29048, 29049, 29050, 29051, 29052, 29053, 29054, 29055, 29056, 29057 were selected from the ORFs having the nucleotide sequence represented by SEQ ID NO: 2091.

The probes having the nucleotide sequence represented by SEQ ID NOS: 29058, 29059, 29060, 29061, 29062, 29063, 29064, 29065, 29066, 29067, 29068 were selected from the ORFs having the nucleotide sequence represented by SEQ ID NO: 2092.

The probes having the nucleotide sequence represented by SEQ ID NOS: 29069, 29070, 29071, 29072, 29073, 29074, 29075, 29076, 29077, 29078, 29079 were selected from the ORFs having the nucleotide sequence represented by SEQ ID NO: 2093.

The probes having the nucleotide sequence represented by SEQ ID NOS: 29080, 29081, 29082, 29083, 29084, 29085, 29086, 29087, 29088, 29089, 29090 were selected from the ORFs having the nucleotide sequence represented by SEQ ID NO: 2094.

The probes having the nucleotide sequence represented by SEQ ID NOS: 29091, 29092, 29093, 29094, 29095, 29096, 29097, 29098, 29099, 29100, 29101 were selected from the ORFs having the nucleotide sequence represented by SEQ ID NO: 2095.

The probes having the nucleotide sequence represented by SEQ ID NOS: 29102, 29103, 29104, 29105, 29106, 29107, 29108, 29109, 29110, 29111, 29112 were selected from the ORFs having the nucleotide sequence represented by SEQ ID NO: 2096.

The probes having the nucleotide sequence represented by SEQ ID NOS: 29113, 29114, 29115, 29116, 29117, 29118, 29119, 29120, 29121, 29122, 29123 were selected from the ORFs having the nucleotide sequence represented by SEQ ID NO: 2097.

The probes having the nucleotide sequence represented by SEQ ID NOS: 29124, 29125, 29126, 29127, 29128, 29129, 29130, 29131, 29132, 29133, 29134 were selected from the ORFs having the nucleotide sequence represented by SEQ ID NO: 2098.

The probes having the nucleotide sequence represented by SEQ ID NOS: 29135, 29136, 29137, 29138, 29139, 29140, 29141, 29142, 29143, 29144, 29145 were selected from the ORFs having the nucleotide sequence represented by SEQ ID NO: 2099.

The probes having the nucleotide sequence represented by SEQ ID NOS: 29146, 29147, 29148, 29149, 29150, 29151, 29152, 29153, 29154, 29155, 29156 were selected from the ORFs having the nucleotide sequence represented by SEQ ID NO: 2100.

The probes having the nucleotide sequence represented by SEQ ID NOS: 29157, 29158, 29159, 29160, 29161, 29162, 29163, 29164, 29165, 29166, 29167 were selected from the ORFs having the nucleotide sequence represented by SEQ ID NO: 2101.

The probes having the nucleotide sequence represented by SEQ ID NOS: 29168, 29169, 29170, 29171, 29172, 29173, 29174, 29175, 29176, 29177, 29178 were selected from the ORFs having the nucleotide sequence represented by SEQ ID NO: 2102.

The probes having the nucleotide sequence represented by SEQ ID NOS: 29179, 29180, 29181, 29182, 29183, 29184, 29185, 29186, 29187, 29188, 29189 were selected from the ORFs having the nucleotide sequence represented by SEQ ID NO: 2103.

The probes having the nucleotide sequence represented by SEQ ID NOS: 29190, 29191, 29192, 29193, 29194, 29195, 29196, 29197, 29198, 29199, 29200 were selected from the ORFs having the nucleotide sequence represented by SEQ ID NO: 2104.

The probes having the nucleotide sequence represented by SEQ ID NOS: 29201, 29202, 29203, 29204, 29205, 29206, 29207, 29208, 29209, 29210, 29211 were selected from the ORFs having the nucleotide sequence represented by SEQ ID NO: 2105.

The probes having the nucleotide sequence represented by. SEQ ID NOS: 29212, 29213, 29214, 29215, 29216, 29217, 29218, 29219, 29220, 29221, 29222 were selected from the ORFs having the nucleotide sequence represented by SEQ ID NO: 2106.

The probes having the nucleotide sequence represented by SEQ ID NOS: 29223, 29224, 29225, 29226, 29227, 29228, 29229, 29230, 29231, 29232, 29233 were selected from the ORFs having the nucleotide sequence represented by SEQ ID NO: 2107.

The probes having the nucleotide sequence represented by SEQ ID NOS: 29234, 29235, 29236, 29237, 29238, 29239, 29240, 29241, 29242, 29243, 29244 were selected from the ORFs having the nucleotide sequence represented by SEQ ID NO: 2108.

The probes having the nucleotide sequence represented by SEQ ID NOS: 29245, 29246, 29247, 29248, 29249, 29250, 29251, 29252, 29253, 29254, 29255 were selected from the ORFs having the nucleotide sequence represented by SEQ ID NO: 2109.

The probes having the nucleotide sequence represented by SEQ ID NOS: 29256, 29257, 29258, 29259, 29260, 29261, 29262, 29263, 29264, 29265, 29266 were selected from the ORFs having the nucleotide sequence represented by SEQ ID NO: 2110.

The probes having the nucleotide sequence represented by SEQ ID NOS: 29267, 29268, 29269, 29270, 29271, 29272, 29273, 29274, 29275, 29276, 29277 were selected from the ORFs having the nucleotide sequence represented by SEQ ID NO: 2111.

The probes having the nucleotide sequence represented by SEQ ID NOS: 29278, 29279, 29280, 29281, 29282, 29283, 29284, 29285, 29286, 29287, 29288 were selected from the ORFs having the nucleotide sequence represented by SEQ ID NO: 2112.

The probes having the nucleotide sequence represented by SEQ ID NOS: 29289, 29290, 29291, 29292, 29293, 29294, 29295, 29296, 29297, 29298, 29299 were selected from the ORFs having the nucleotide sequence represented by SEQ ID NO: 2113.

The probes having the nucleotide sequence represented by SEQ ID NOS: 29300, 29301, 29302, 29303, 29304, 29305, 29306, 29307, 29308, 29309, 29310 were selected from the ORFs having the nucleotide sequence represented by SEQ ID NO: 2114.

The probes having the nucleotide sequence represented by SEQ ID NOS: 29311, 29312, 29313, 29314, 29315, 29316, 29317, 29318, 29319, 29320, 29321 were selected from the ORFs having the nucleotide sequence represented by SEQ ID NO: 2115.

The probes having the nucleotide sequence represented by SEQ ID NOS: 29322, 29323, 29324, 29325, 29326, 29327, 29328, 29329, 29330, 29331, 29332 were selected from the ORFs having the nucleotide sequence represented by SEQ ID NO: 2116.

The probes having the nucleotide sequence represented by SEQ ID NOS: 29333, 29334, 29335, 29336, 29337, 29338, 29339, 29340, 29341, 29342, 29343 were selected from the ORFs having the nucleotide sequence represented by SEQ ID NO: 2117.

The probes having the nucleotide sequence represented by SEQ ID NOS: 29344, 29345, 29346, 29347, 29348, 29349, 29350, 29351, 29352, 29353, 29354 were selected from the ORFs having the nucleotide sequence represented by SEQ ID NO: 2118.

The probes having the nucleotide sequence represented by SEQ ID NOS: 29355, 29356, 29357, 29358, 29359, 29360, 29361, 29362, 29363, 29364, 29365 were selected from the ORFs having the nucleotide sequence represented by SEQ ID NO: 2119.

The probes having the nucleotide sequence represented by SEQ ID NOS: 29366, 29367, 29368, 29369, 29370, 29371, 29372, 29373, 29374, 29375, 29376 were selected from the ORFs having the nucleotide sequence represented by SEQ ID NO: 2120.

The probes having the nucleotide sequence represented by SEQ ID NOS: 29377, 29378, 29379, 29380, 29381, 29382, 29383, 29384, 29385, 29386, 29387 were selected from the ORFs having the nucleotide sequence represented by SEQ ID NO: 2121.

The probes having the nucleotide sequence represented by SEQ ID NOS: 29388, 29389, 29390, 29391, 29392, 29393, 29394, 29395, 29396, 29397, 29398 were selected from the ORFs having the nucleotide sequence represented by SEQ ID NO: 2122.

The probes having the nucleotide sequence represented by SEQ ID NOS: 29399, 29400, 29401, 29402, 29403, 29404, 29405, 29406, 29407, 29408, 29409 were selected from the ORFs having the nucleotide sequence represented by SEQ ID NO: 2123.

The probes having the nucleotide sequence represented by SEQ ID NOS: 29410, 29411, 29412, 29413, 29414, 29415, 29416, 29417, 29418, 29419, 29420 were selected from the ORFs having the nucleotide sequence represented by SEQ ID NO: 2124.

The probes having the nucleotide sequence represented by SEQ ID NOS: 29421, 29422, 29423, 29424, 29425, 29426, 29427, 29428, 29429, 29430, 29431 were selected from the ORFs having the nucleotide sequence represented by SEQ ID NO: 2125.

The probes having the nucleotide sequence represented by SEQ ID NOS: 29432, 29433, 29434, 29435, 29436, 29437, 29438, 29439, 29440, 29441, 29442 were selected from the ORFs having the nucleotide sequence represented by SEQ ID NO: 2126.

The probes having the nucleotide sequence represented by SEQ ID NOS: 29443, 29444, 29445, 29446, 29447, 29448, 29449, 29450, 29451, 29452, 29453 were selected from the ORFs having the nucleotide sequence represented by SEQ ID NO: 2127.

The probes having the nucleotide sequence represented by SEQ ID NOS: 29454, 29455, 29456, 29457, 29458, 29459, 29460, 29461, 29462, 29463, 29464 were selected from the ORFs having the nucleotide sequence represented by SEQ ID NO: 2128.

The probes having the nucleotide sequence represented by SEQ ID NOS: 29465, 29466, 29467, 29468, 29469, 29470, 29471, 29472, 29473, 29474, 29475 were selected from the ORFs having the nucleotide sequence represented by SEQ ID NO: 2129.

The probes having the nucleotide sequence represented by SEQ ID NOS: 29476, 29477, 29478, 29479, 29480, 29481, 29482, 29483, 29484, 29485, 29486 were selected from the ORFs having the nucleotide sequence represented by SEQ ID NO: 2130.

The probes having the nucleotide sequence represented by SEQ ID NOS: 29487, 29488, 29489, 29490, 29491, 29492, 29493, 29494, 29495, 29496, 29497 were selected from the ORFs having the nucleotide sequence represented by SEQ ID NO: 2131.

The probes having the nucleotide sequence represented by SEQ ID NOS: 29498, 29499, 29500, 29501, 29502, 29503, 29504, 29505, 29506, 29507, 29508 were selected from the ORFs having the nucleotide sequence represented by SEQ ID NO: 2132.

The probes having the nucleotide sequence represented by SEQ ID NOS: 29509, 29510, 29511, 29512, 29513, 29514, 29515, 29516, 29517, 29518, 29519 were selected from the ORFs having the nucleotide sequence represented by SEQ ID NO: 2133.

The probes having the nucleotide sequence represented by SEQ ID NOS: 29520, 29521, 29522, 29523, 29524, 29525, 29526, 29527, 29528, 29529, 29530 were selected from the ORFs having the nucleotide sequence represented by SEQ ID NO: 2134.

The probes having the nucleotide sequence represented by SEQ ID NOS: 29531, 29532, 29533, 29534, 29535, 29536, 29537, 29538, 29539, 29540, 29541 were selected from the ORFs having the nucleotide sequence represented by SEQ ID NO: 2135.

The probes having the nucleotide sequence represented by SEQ ID NOS: 29542, 29543, 29544, 29545, 29546, 29547, 29548, 29549, 29550, 29551, 29552 were selected from the ORFs having the nucleotide sequence represented by SEQ ID NO: 2136.

The probes having the nucleotide sequence represented by SEQ ID NOS: 29553, 29554, 29555, 29556, 29557, 29558, 29559, 29560, 29561, 29562, 29563 were selected from the ORFs having the nucleotide sequence represented by SEQ ID NO: 2137.

The probes having the nucleotide sequence represented by SEQ ID NOS: 29564, 29565, 29566, 29567, 29568, 29569, 29570, 29571, 29572, 29573, 29574 were selected from the ORFs having the nucleotide sequence represented by SEQ ID NO: 2138.

The probes having the nucleotide sequence represented by SEQ ID NOS: 29575, 29576, 29577, 29578, 29579, 29580, 29581, 29582, 29583, 29584, 29585 were selected from the ORFs having the nucleotide sequence represented by SEQ ID NO: 2139.

The probes having the nucleotide sequence represented by SEQ ID NOS: 29586, 29587, 29588, 29589, 29590, 29591, 29592, 29593, 29594, 29595, 29596 were selected from the ORFs having the nucleotide sequence represented by SEQ ID NO: 2140.

The probes having the nucleotide sequence represented by SEQ ID NOS: 29597, 29598, 29599, 29600, 29601, 29602, 29603, 29604, 29605, 29606, 29607 were selected from the ORFs having the nucleotide sequence represented by SEQ ID NO: 2141.

The probes having the nucleotide sequence represented by SEQ ID NOS: 29608, 29609, 29610, 29611, 29612, 29613, 29614, 29615, 29616, 29617, 29618 were selected from the ORFs having the nucleotide sequence represented by SEQ ID NO: 2142.

The probes having the nucleotide sequence represented by SEQ ID NOS: 29619, 29620, 29621, 29622, 29623, 29624, 29625, 29626, 29627, 29628, 29629 were selected from the ORFs having the nucleotide sequence represented by SEQ ID NO: 2143.

The probes having the nucleotide sequence represented by SEQ ID NOS: 29630, 29631, 29632, 29633, 29634, 29635, 29636, 29637, 29638, 29639, 29640 were selected from the ORFs having the nucleotide sequence represented by SEQ ID NO: 2144.

The probes having the nucleotide sequence represented by SEQ ID NOS: 29641, 29642, 29643, 29644, 29645, 29646, 29647, 29648, 29649, 29650, 29651 were selected from the ORFs having the nucleotide sequence represented by SEQ ID NO: 2145.

The probes having the nucleotide sequence represented by SEQ ID NOS: 29652, 29653, 29654, 29655, 29656, 29657, 29658, 29659, 29660, 29661, 29662 were selected from the ORFs having the nucleotide sequence represented by SEQ ID NO: 2146.

The probes having the nucleotide sequence represented by SEQ ID NOS: 29663, 29664, 29665, 29666, 29667, 29668, 29669, 29670, 29671, 29672, 29673 were selected from the ORFs having the nucleotide sequence represented by SEQ ID NO: 2147.

The probes having the nucleotide sequence represented by SEQ ID NOS: 29674, 29675, 29676, 29677, 29678, 29679, 29680, 29681, 29682, 29683, 29684 were selected from the ORFs having the nucleotide sequence represented by SEQ ID NO: 2148.

The probes having the nucleotide sequence represented by SEQ ID NOS: 29685, 29686, 29687, 29688, 29689, 29690, 29691, 29692, 29693, 29694, 29695 were selected from the ORFs having the nucleotide sequence represented by SEQ ID NO: 2149.

The probes having the nucleotide sequence represented by SEQ ID NOS: 29696, 29697, 29698, 29699, 29700, 29701, 29702, 29703, 29704, 29705, 29706 were selected from the ORFs having the nucleotide sequence represented by SEQ ID NO: 2150.

The probes having the nucleotide sequence represented by SEQ ID NOS: 29707, 29708, 29709, 29710, 29711, 29712, 29713, 29714, 29715, 29716, 29717 were selected from the ORFs having the nucleotide sequence represented by SEQ ID NO: 2151.

The probes having the nucleotide sequence represented by SEQ ID NOS: 29718, 29719, 29720, 29721, 29722, 29723, 29724, 29725, 29726, 29727, 29728 were selected from the ORFs having the nucleotide sequence represented by SEQ ID NO: 2152.

The probes having the nucleotide sequence represented by SEQ ID NOS: 29729, 29730, 29731, 29732, 29733, 29734, 29735, 29736, 29737, 29738, 29739 were selected from the ORFs having the nucleotide sequence represented by SEQ ID NO: 2153.

The probes having the nucleotide sequence represented by SEQ ID NOS: 29740, 29741, 29742, 29743, 29744, 29745, 29746, 29747, 29748, 29749, 29750 were selected from the ORFs having the nucleotide sequence represented by SEQ ID NO: 2154.

The probes having the nucleotide sequence represented by SEQ ID NOS: 29751, 29752, 29753, 29754, 29755, 29756, 29757, 29758, 29759, 29760, 29761 were selected from the ORFs having the nucleotide sequence represented by SEQ ID NO: 2155.

The probes having the nucleotide sequence represented by SEQ ID NOS: 29762, 29763, 29764, 29765, 29766, 29767, 29768, 29769, 29770, 29771, 29772 were selected from the ORFs having the nucleotide sequence represented by SEQ ID NO: 2156.

The probes having the nucleotide sequence represented by SEQ ID NOS: 29773, 29774, 29775, 29776, 29777, 29778, 29779, 29780, 29781, 29782, 29783 were selected from the ORFs having the nucleotide sequence represented by SEQ ID NO: 2157.

The probes having the nucleotide sequence represented by SEQ ID NOS: 29784, 29785, 29786, 29787, 29788, 29789, 29790, 29791, 29792, 29793, 29794 were selected from the ORFs having the nucleotide sequence represented by SEQ ID NO: 2158.

The probes having the nucleotide sequence represented by SEQ ID NOS: 29795, 29796, 29797, 29798, 29799, 29800, 29801, 29802, 29803, 29804, 29805 were selected from the ORFs having the nucleotide sequence represented by SEQ ID NO: 2159.

The probes having the nucleotide sequence represented by SEQ ID NOS: 29806, 29807, 29808, 29809, 29810, 29811, 29812, 29813, 29814, 29815, 29816 were selected from the ORFs having the nucleotide sequence represented by SEQ ID NO: 2160.

The probes having the nucleotide sequence represented by SEQ ID NOS: 29817, 29818, 29819, 29820, 29821, 29822, 29823, 29824, 29825, 29826, 29827 were selected from the ORFs having the nucleotide sequence represented by SEQ ID NO: 2161.

The probes having the nucleotide sequence represented by SEQ ID NOS: 29828, 29829, 29830, 29831, 29832, 29833, 29834, 29835, 29836, 29837, 29838 were selected from the ORFs having the nucleotide sequence represented by SEQ ID NO: 2162.

The probes having the nucleotide sequence represented by SEQ ID NOS: 29839, 29840, 29841, 29842, 29843, 29844, 29845, 29846, 29847, 29848, 29849 were selected from the ORFs having the nucleotide sequence represented by SEQ ID NO: 2163.

The probes having the nucleotide sequence represented by SEQ ID NOS: 29850, 29851, 29852, 29853, 29854, 29855, 29856, 29857, 29858, 29859, 29860 were selected from the ORFs having the nucleotide sequence represented by SEQ ID NO: 2164.

The probes having the nucleotide sequence represented by SEQ ID NOS: 29861, 29862, 29863, 29864, 29865, 29866, 29867, 29868, 29869, 29870, 29871 were selected from the ORFs having the nucleotide sequence represented by SEQ ID NO: 2165.

The probes having the nucleotide sequence represented by SEQ ID NOS: 29872, 29873, 29874, 29875, 29876, 29877, 29878, 29879, 29880, 29881, 29882 were selected from the ORFs having the nucleotide sequence represented by SEQ ID NO: 2166.

The probes having the nucleotide sequence represented by SEQ ID NOS: 29883, 29884, 29885, 29886, 29887, 29888, 29889, 29890, 29891, 29892, 29893 were selected from the ORFs having the nucleotide sequence represented by SEQ ID NO: 2167.

The probes having the nucleotide sequence represented by SEQ ID NOS: 29894, 29895, 29896, 29897, 29898, 29899, 29900, 29901, 29902, 29903, 29904 were selected from the ORFs having the nucleotide sequence represented by SEQ ID NO: 2168.

The probes having the nucleotide sequence represented by SEQ ID NOS: 29905, 29906, 29907, 29908, 29909, 29910, 29911, 29912, 29913, 29914, 29915 were selected from the ORFs having the nucleotide sequence represented by SEQ ID NO: 2169.

The probes having the nucleotide sequence represented by SEQ ID NOS: 29916, 29917, 29918, 29919, 29920, 29921, 29922, 29923, 29924, 29925, 29926 were selected from the ORFs having the nucleotide sequence represented by SEQ ID NO: 2170.

The probes having the nucleotide sequence represented by SEQ ID NOS: 29927, 29928, 29929, 29930, 29931, 29932, 29933, 29934, 29935, 29936, 29937 were selected from the ORFs having the nucleotide sequence represented by SEQ ID NO: 2171.

The probes having the nucleotide sequence represented by SEQ ID NOS: 29938, 29939, 29940, 29941, 29942, 29943, 29944, 29945, 29946, 29947, 29948 were selected from the ORFs having the nucleotide sequence represented by SEQ ID NO: 2172.

The probes having the nucleotide sequence represented by SEQ ID NOS: 29949, 29950, 29951, 29952, 29953, 29954, 29955, 29956, 29957, 29958, 29959 were selected from the ORFs having the nucleotide sequence represented by SEQ ID NO: 2173.

The probes having the nucleotide sequence represented by SEQ ID NOS: 29960, 29961, 29962, 29963, 29964, 29965, 29966, 29967, 29968, 29969, 29970 were selected from the ORFs having the nucleotide sequence represented by SEQ ID NO: 2174.

The probes having the nucleotide sequence represented by SEQ ID NOS: 29971, 29972, 29973, 29974, 29975, 29976, 29977, 29978, 29979, 29980, 29981 were selected from the ORFs having the nucleotide sequence represented by SEQ ID NO: 2175.

The probes having the nucleotide sequence represented by SEQ ID NOS: 29982, 29983, 29984, 29985, 29986, 29987, 29988, 29989, 29990, 29991, 29992 were selected from the ORFs having the nucleotide sequence represented by SEQ ID NO: 2176.

The probes having the nucleotide sequence represented by SEQ ID NOS: 29993, 29994, 29995, 29996, 29997, 29998, 29999, 30000, 30001, 30002, 30003 were selected from the ORFs having the nucleotide sequence represented by SEQ ID NO: 2177.

The probes having the nucleotide sequence represented by SEQ ID NOS: 30004, 30005, 30006, 30007, 30008, 30009, 30010, 30011, 30012, 30013, 30014 were selected from the ORFs having the nucleotide sequence represented by SEQ ID NO: 2178.

The probes having the nucleotide sequence represented by SEQ ID NOS: 30015, 30016, 30017, 30018, 30019, 30020, 30021, 30022, 30023, 30024, 30025 were selected from the ORFs having the nucleotide sequence represented by SEQ ID NO: 2179.

The probes having the nucleotide sequence represented by SEQ ID NOS: 30026, 30027, 30028, 30029, 30030, 30031, 30032, 30033, 30034, 30035, 30036 were selected from the ORFs having the nucleotide sequence represented by SEQ ID NO: 2180.

The probes having the nucleotide sequence represented by SEQ ID NOS: 30037, 30038, 30039, 30040, 30041, 30042, 30043, 30044, 30045, 30046, 30047 were selected from the ORFs having the nucleotide sequence represented by SEQ ID NO: 2181.

The probes having the nucleotide sequence represented by SEQ ID NOS: 30048, 30049, 30050, 30051, 30052, 30053, 30054, 30055, 30056, 30057, 30058were selected from the ORFs having the nucleotide sequence represented by SEQ ID NO: 2182.

The probes having the nucleotide sequence represented by SEQ ID NOS: 30059, 30060, 30061, 30062, 30063, 30064, 30065, 30066, 30067, 30068, 30069 were selected from the ORFs having the nucleotide sequence represented by SEQ ID NO: 2183.

The probes having the nucleotide sequence represented by SEQ ID NOS: 30070, 30071, 30072, 30073, 30074, 30075, 30076, 30077, 30078, 30079, 30080 were selected from the ORFs having the nucleotide sequence represented by SEQ ID NO: 2184.

The probes having the nucleotide sequence represented by SEQ ID NOS: 30081, 30082, 30083, 30084, 30085, 30086, 30087, 30088, 30089, 30090, 30091 were selected from the ORFs having the nucleotide sequence represented by SEQ ID NO: 2185.

The probes having the nucleotide sequence represented by SEQ ID NOS: 30092, 30093, 30094, 30095, 30096, 30097, 30098, 30099, 30100, 30101, 30102 were selected from the ORFs having the nucleotide sequence represented by SEQ ID NO: 2186.

The probes having the nucleotide sequence represented by SEQ ID NOS: 30103, 30104, 30105, 30106, 30107, 30108, 30109, 30110, 30111, 30112, 30113 were selected from the ORFs having the nucleotide sequence represented by SEQ ID NO: 2187.

The probes having the nucleotide sequence represented by SEQ ID NOS: 30114, 30115, 30116, 30117, 30118, 30119, 30120, 30121, 30122, 30123, 30124 were selected from the ORFs having the nucleotide sequence represented by SEQ ID NO: 2188.

The probes having the nucleotide sequence represented by SEQ ID NOS: 30125, 30126, 30127, 30128, 30129, 30130, 30131, 30132, 30133, 30134, 30135 were selected from the ORFs having the nucleotide sequence represented by SEQ ID NO: 2189.

The probes having the nucleotide sequence represented by SEQ ID NOS: 30136, 30137, 30138, 30139, 30140, 30141, 30142, 30143, 30144, 30145, 30146 were selected from the ORFs having the nucleotide sequence represented by SEQ ID NO: 2190.

The probes having the nucleotide sequence represented by SEQ ID NOS: 30147, 30148, 30149, 30150, 30151, 30152, 30153, 30154, 30155, 30156, 30157 were selected from the ORFs having the nucleotide sequence represented by SEQ ID NO: 2191.

The probes having the nucleotide sequence represented by SEQ ID NOS: 30158, 30159, 30160, 30161, 30162, 30163, 30164, 30165, 30166, 30167, 30168 were selected from the ORFs having the nucleotide sequence represented by SEQ ID NO: 2192.

The probes having the nucleotide sequence represented by SEQ ID NOS: 30169, 30170, 30171, 30172, 30173, 30174, 30175, 30176, 30177, 30178, 30179 were selected from the ORFs having the nucleotide sequence represented by SEQ ID NO: 2193.

The probes having the nucleotide sequence represented by SEQ ID NOS: 30180, 30181, 30182, 30183, 30184, 30185, 30186, 30187, 30188, 30189, 30190 were selected from the ORFs having the nucleotide sequence represented by SEQ ID NO: 2194.

The probes having the nucleotide sequence represented by SEQ ID NOS: 30191, 30192, 30193, 30194, 30195, 30196, 30197, 30198, 30199, 30200, 30201 were selected from the ORFs having the nucleotide sequence represented by SEQ ID NO: 2195.

The probes having the nucleotide sequence represented by SEQ ID NOS: 30202, 30203, 30204, 30205, 30206, 30207, 30208, 30209, 30210, 30211, 30212 were selected from the ORFs having the nucleotide sequence represented by SEQ ID NO: 2196.

The probes having the nucleotide sequence represented by SEQ ID NOS: 30213, 30214, 30215, 30216, 30217, 30218, 30219, 30220, 30221, 30222, 30223 were selected from the ORFs having the nucleotide sequence represented by SEQ ID NO: 2197.

The probes having the nucleotide sequence represented by SEQ ID NOS: 30224, 30225, 30226, 30227, 30228, 30229, 30230, 30231, 30232, 30233, 30234 were selected from the ORFs having the nucleotide sequence represented by SEQ ID NO: 2198.

The probes having the nucleotide sequence represented by SEQ ID NOS: 30235, 30236, 30237, 30238, 30239, 30240, 30241, 30242, 30243, 30244, 30245 were selected from the ORFs having the nucleotide sequence represented by SEQ ID NO: 2199.

The probes having the nucleotide sequence represented by SEQ ID NOS: 30246, 30247, 30248, 30249, 30250, 30251, 30252, 30253, 30254, 30255, 30256 were selected from the ORFs having the nucleotide sequence represented by SEQ ID N O: 2200.

The probes having the nucleotide sequence represented by SEQ ID NOS: 30257, 30258, 30259, 30260, 30261, 30262, 30263, 30264, 30265, 30266, 30267, 30268, 30269, 30270, 30271, 30272, 30273, 30274, 30275, 30276, 30277, 30278 were selected from the ORFs having the nucleotide sequence represented by SEQ ID NO: 2201.

The probes having the nucleotide sequence represented by SEQ ID NOS: 30279, 30280, 30281, 30282, 30283, 30284, 30285, 30286, 30287, 30288, 30289 were selected from the ORFs having the nucleotide sequence represented by SEQ ID NO: 2202.

The probes having the nucleotide sequence represented by SEQ ID NOS: 30290, 30291, 30292, 30293, 30294, 30295, 30296, 30297, 30298, 30299, 30300 were selected from the ORFs having the nucleotide sequence represented by SEQ ID NO: 2203.

The probes having the nucleotide sequence represented by SEQ ID NOS: 30301, 30302, 30303, 30304, 30305, 30306, 30307, 30308, 30309, 30310, 30311 were selected from the ORFs having the nucleotide sequence represented by SEQ ID NO: 2204.

The probes having the nucleotide sequence represented by SEQ ID NOS: 30312, 30313, 30314, 30315, 30316, 30317, 30318, 30319, 30320, 30321, 30322 were selected from the ORFs having the nucleotide sequence represented by SEQ ID NO: 2205.

The probes having the nucleotide sequence represented by SEQ ID NOS: 30323, 30324, 30325, 30326, 30327, 30328, 30329, 30330, 30331, 30332, 30333 were selected from the ORFs having the nucleotide sequence represented by SEQ ID NO: 2206.

The probes having the nucleotide sequence represented by SEQ ID NOS: 30334, 30335, 30336, 30337, 30338, 30339, 30340, 30341, 30342, 30343, 30344 were selected from the ORFs having the nucleotide sequence represented by SEQ ID NO: 2207.

The probes having the nucleotide sequence represented by SEQ ID NOS: 30345, 30346, 30347, 30348, 30349, 30350, 30351, 30352, 30353, 30354, 30355 were selected from the ORFs having the nucleotide sequence represented by SEQ ID NO: 2208.

The probes having the nucleotide sequence represented by SEQ ID NOS: 30356, 30357, 30358, 30359, 30360, 30361, 30362, 30363, 30364, 30365, 30366 were selected from the ORFs having the nucleotide sequence represented by SEQ ID NO: 2209.

The probes having the nucleotide sequence represented by SEQ ID NOS: 30367, 30368, 30369, 30370, 30371, 30372, 30373, 30374, 30375, 30376, 30377 were selected from the ORFs having the nucleotide sequence represented by SEQ ID NO: 2210.

The probes having the nucleotide sequence represented by SEQ ID NOS: 30378, 30379, 30380, 30381, 30382, 30383, 30384, 30385, 30386, 30387, 30388 were selected from the ORFs having the nucleotide sequence represented by SEQ ID NO: 2211.

The probes having the nucleotide sequence represented by SEQ ID NOS: 30389, 30390, 30391, 30392, 30393, 30394, 30395, 30396, 30397, 30398, 30399 were selected from the ORFs having the nucleotide sequence represented by SEQ ID NO: 2212.

The probes having the nucleotide sequence represented by SEQ ID NOS: 30400, 30401, 30402, 30403, 30404, 30405, 30406, 30407, 30408, 30409, 30410 were selected from the ORFs having the nucleotide sequence represented by SEQ ID NO: 2213.

The probes having the nucleotide sequence represented by SEQ ID NOS: 30411, 30412, 30413, 30414, 30415, 30416, 30417, 30418, 30419, 30420, 30421 were selected from the ORFs having the nucleotide sequence represented by SEQ ID NO: 2214.

The probes having the nucleotide sequence represented by SEQ ID NOS: 30422, 30423, 30424, 30425, 30426, 30427, 30428, 30429, 30430, 30431, 30432 were selected from the ORFs having the nucleotide sequence represented by SEQ ID NO: 2215.

The probes having the nucleotide sequence represented by SEQ ID NOS: 30433, 30434, 30435, 30436, 30437, 30438, 30439, 30440, 30441, 30442, 30443 were selected from the ORFs having the nucleotide sequence represented by SEQ ID NO: 2216.

The probes having the nucleotide sequence represented by SEQ ID NOS: 30444, 30445, 30446, 30447, 30448, 30449, 30450, 30451, 30452, 30453, 30454 were selected from the ORFs having the nucleotide sequence represented by SEQ ID NO: 2217.

The probes having the nucleotide sequence represented by SEQ ID NOS: 30455, 30456, 30457, 30458, 30459, 30460, 30461, 30462, 30463, 30464, 30465 were selected from the ORFs having the nucleotide sequence represented by SEQ ID NO: 2218.

The probes having the nucleotide sequence represented by SEQ ID NOS: 30466, 30467, 30468, 30469, 30470, 30471, 30472, 30473, 30474, 30475, 30476 were selected from the ORFs having the nucleotide sequence represented by SEQ ID NO: 2219.

The probes having the nucleotide sequence represented by SEQ ID NOS: 30477, 30478, 30479, 30480, 30481, 30482, 30483, 30484, 30485, 30486, 30487 were selected from the ORFs having the nucleotide sequence represented by SEQ ID NO: 2220.

The probes having the nucleotide sequence represented by SEQ ID NOS: 30488, 30489, 30490, 30491, 30492, 30493, 30494, 30495, 30496, 30497, 30498 were selected from the ORFs having the nucleotide sequence represented by SEQ ID NO: 2221.

The probes having the nucleotide sequence represented by SEQ ID NOS: 30499, 30500, 30501, 30502, 30503, 30504, 30505, 30506, 30507, 30508, 30509 were selected from the ORFs having the nucleotide sequence represented by SEQ ID NO: 2222.

The probes having the nucleotide sequence represented by SEQ ID NOS: 30510, 30511, 30512, 30513, 30514, 30515, 30516, 30517, 30518, 30519, 30520 were selected from the ORFs having the nucleotide sequence represented by SEQ ID NO: 2223.

The probes having the nucleotide sequence represented by SEQ ID NOS: 30521, 30522, 30523, 30524, 30525, 30526, 30527, 30528, 30529, 30530, 30531 were selected from the ORFs having the nucleotide sequence represented by SEQ ID NO: 2224.

The probes having the nucleotide sequence represented by SEQ ID NOS: 30532, 30533, 30534, 30535, 30536, 30537, 30538, 30539, 30540, 30541, 30542 were selected from the ORFs having the nucleotide sequence represented by SEQ ID NO: 2225.

The probes having the nucleotide sequence represented by SEQ ID NOS: 30543, 30544, 30545, 30546, 30547, 30548, 30549, 30550, 30551, 30552, 30553 were selected from the ORFs having the nucleotide sequence represented by SEQ ID NO: 2226.

The probes having the nucleotide sequence represented by SEQ ID NOS: 30554, 30555, 30556, 30557, 30558, 30559, 30560, 30561, 30562, 30563, 30564 were selected from the ORFs having the nucleotide sequence represented by SEQ ID NO: 2227.

The probes having the nucleotide sequence represented by SEQ ID NOS: 30565, 30566, 30567, 30568, 30569, 30570, 30571, 30572, 30573, 30574, 30575 were selected from the ORFs having the nucleotide sequence represented by SEQ ID NO: 2228.

The probes having the nucleotide sequence represented by SEQ ID NOS: 30576, 30577, 30578, 30579, 30580, 30581, 30582, 30583, 30584, 30585, 30586 were selected from the ORFs having the nucleotide sequence represented by SEQ ID NO: 2229.

The probes having the nucleotide sequence represented by SEQ ID NOS: 30587, 30588, 30589, 30590, 30591, 30592, 30593, 30594, 30595, 30596, 30597 were selected from the ORFs having the nucleotide sequence represented by SEQ ID NO: 2230.

The probes having the nucleotide sequence represented by SEQ ID NOS: 30598, 30599, 30600, 30601, 30602, 30603, 30604, 30605, 30606, 30607, 30608 were selected from the ORFs having the nucleotide sequence represented by SEQ ID NO: 2231.

The probes having the nucleotide sequence represented by SEQ ID NOS: 30609, 30610, 30611, 30612, 30613, 30614, 30615, 30616, 30617, 30618, 30619 were selected from the ORFs having the nucleotide sequence represented by SEQ ID NO: 2232.

The probes having the nucleotide sequence represented by SEQ ID NOS: 30620, 30621, 30622, 30623, 30624, 30625, 30626, 30627, 30628, 30629, 30630 were selected from the ORFs having the nucleotide sequence represented by SEQ ID NO: 2233.

The probes having the nucleotide sequence represented by SEQ ID NOS: 30631, 30632, 30633, 30634, 30635, 30636, 30637, 30638, 30639, 30640, 30641 were selected from the ORFs having the nucleotide sequence represented by SEQ ID NO: 2234.

The probes having the nucleotide sequence represented by SEQ ID NOS: 30642, 30643, 30644, 30645, 30646, 30647, 30648, 30649, 30650, 30651, 30652 were selected from the ORFs having the nucleotide sequence represented by SEQ ID NO: 2235.

The probes having the nucleotide sequence represented by SEQ ID NOS: 30653, 30654, 30655, 30656, 30657, 30658, 30659, 30660, 30661, 30662, 30663 were selected from the ORFs having the nucleotide sequence represented by SEQ ID NO: 2236.

The probes having the nucleotide sequence represented by SEQ ID NOS: 30664, 30665, 30666, 30667, 30668, 30669, 30670, 30671, 30672, 30673, 30674 were selected from the ORFs having the nucleotide sequence represented by SEQ ID NO: 2237.

The probes having the nucleotide sequence represented by SEQ ID NOS: 30675, 30676, 30677, 30678, 30679, 30680, 30681, 30682, 30683, 30684, 30685 were selected from the ORFs having the nucleotide sequence represented by SEQ ID NO: 2238.

The probes having the nucleotide sequence represented by SEQ ID NOS: 30686, 30687, 30688, 30689, 30690, 30691, 30692, 30693, 30694, 30695, 30696 were selected from the ORFs having the nucleotide sequence represented by SEQ ID NO: 2239.

The probes having the nucleotide sequence represented by SEQ ID NOS: 30697, 30698, 30699, 30700, 30701, 30702, 30703, 30704, 30705, 30706, 30707 were selected from the ORFs having the nucleotide sequence represented by SEQ ID NO: 2240.

The probes having the nucleotide sequence represented by SEQ ID NOS: 30708, 30709, 30710, 30711, 30712, 30713, 30714, 30715, 30716, 30717, 30718 were selected from the ORFs having the nucleotide sequence represented by SEQ ID NO: 2241.

The probes having the nucleotide sequence represented by SEQ ID NOS: 30719, 30720, 30721, 30722, 30723, 30724, 30725, 30726, 30727, 30728, 30729 were selected from the ORFs having the nucleotide sequence represented by SEQ ID NO: 2242.

The probes having the nucleotide sequence represented by SEQ ID NOS: 30730, 30731, 30732, 30733, 30734, 30735, 30736, 30737, 30738, 30739, 30740 were selected from the ORFs having the nucleotide sequence represented by SEQ ID NO: 2243.

The probes having the nucleotide sequence represented by SEQ ID NOS: 30741, 30742, 30743, 30744, 30745, 30746, 30747, 30748, 30749, 30750, 30751 were selected from the ORFs having the nucleotide sequence represented by SEQ ID NO: 2244.

The probes having the nucleotide sequence represented by SEQ ID NOS: 30752, 30753, 30754, 30755, 30756, 30757, 30758, 30759, 30760, 30761, 30762 were selected from the ORFs having the nucleotide sequence represented by SEQ ID NO: 2245.

The probes having the nucleotide sequence represented by SEQ ID NOS: 30763, 30764, 30765, 30766, 30767, 30768, 30769, 30770, 30771, 30772, 30773 were selected from the ORFs having the nucleotide sequence represented by SEQ ID NO: 2246.

The probes having the nucleotide sequence represented by SEQ ID NOS: 30774, 30775, 30776, 30777, 30778, 30779, 30780, 30781, 30782, 30783, 30784 were selected from the ORFs having the nucleotide sequence represented by SEQ ID NO: 2247.

The probes having the nucleotide sequence represented by SEQ ID NOS: 30785, 30786, 30787, 30788, 30789, 30790, 30791, 30792, 30793, 30794, 30795 were selected from the ORFs having the nucleotide sequence represented by SEQ ID NO: 2248.

The probes having the nucleotide sequence represented by SEQ ID NOS: 30796, 30797, 30798, 30799, 30800, 30801, 30802, 30803, 30804, 30805, 30806 were selected from the ORFs having the nucleotide sequence represented by SEQ ID NO: 2249.

The probes having the nucleotide sequence represented by SEQ ID NOS: 30807, 30808, 30809, 30810, 30811, 30812, 30813, 30814, 30815, 30816, 30817 were selected from the ORFs having the nucleotide sequence represented by SEQ ID NO: 2250.

The probes having the nucleotide sequence represented by SEQ ID NOS: 30818, 30819, 30820, 30821, 30822, 30823, 30824, 30825, 30826, 30827, 30828 were selected from the ORFs having the nucleotide sequence represented by SEQ ID NO: 2251.

The probes having the nucleotide sequence represented by SEQ ID NOS: 30829, 30830, 30831, 30832, 30833, 30834, 30835, 30836, 30837, 30838, 30839 were selected from the ORFs having the nucleotide sequence represented by SEQ ID NO: 2252.

The probes having the nucleotide sequence represented by SEQ ID NOS: 30840, 30841, 30842, 30843, 30844, 30845, 30846, 30847, 30848, 30849, 30850 were selected from the ORFs having the nucleotide sequence represented by SEQ ID NO: 2253.

The probes having the nucleotide sequence represented by SEQ ID NOS: 30851, 30852, 30853, 30854, 30855, 30856, 30857, 30858, 30859, 30860, 30861 were selected from the ORFs having the nucleotide sequence represented by SEQ ID NO: 2254.

The probes having the nucleotide sequence represented by SEQ ID NOS: 30862, 30863, 30864, 30865, 30866, 30867, 30868, 30869, 30870, 30871, 30872 were selected from the ORFs having the nucleotide sequence represented by SEQ ID NO: 2255.

The probes having the nucleotide sequence represented by SEQ ID NOS: 30873, 30874, 30875, 30876, 30877, 30878, 30879, 30880, 30881, 30882, 30883 were selected from the ORFs having the nucleotide sequence represented by SEQ ID NO: 2256.

The probes having the nucleotide sequence represented by SEQ ID NOS: 30884, 30885, 30886, 30887, 30888, 30889, 30890, 30891, 30892, 30893, 30894 were selected from the ORFs having the nucleotide sequence represented by SEQ ID NO: 2257.

The probes having the nucleotide sequence represented by SEQ ID NOS: 30895, 30896, 30897, 30898, 30899, 30900, 30901, 30902, 30903, 30904, 30905 were selected from the ORFs having the nucleotide sequence represented by SEQ ID NO: 2258.

The probes having the nucleotide sequence represented by SEQ ID NOS: 30906, 30907, 30908, 30909, 30910, 30911, 30912, 30913, 30914, 30915, 30916 were selected from the ORFs having the nucleotide sequence represented by SEQ ID NO: 2259.

The probes having the nucleotide sequence represented by SEQ ID NOS: 30917, 30918, 30919, 30920, 30921, 30922, 30923, 30924, 30925, 30926, 30927 were selected from the ORFs having the nucleotide sequence represented by SEQ ID NO: 2260.

The probes having the nucleotide sequence represented by SEQ ID NOS: 30928, 30929, 30930, 30931, 30932, 30933, 30934, 30935, 30936, 30937, 30938 were selected from the ORFs having the nucleotide sequence represented by SEQ ID NO: 2261.

The probes having the nucleotide sequence represented by SEQ ID NOS: 30939, 30940, 30941, 30942, 30943, 30944, 30945, 30946, 30947, 30948, 30949 were selected from the ORFs having the nucleotide sequence represented by SEQ ID NO: 2262.

The probes having the nucleotide sequence represented by SEQ ID NOS: 30950, 30951, 30952, 30953, 30954, 30955, 30956, 30957, 30958, 30959, 309.60 were selected from the ORFs having the nucleotide sequence represented by SEQ ID NO: 2263.

The probes having the nucleotide sequence represented by SEQ ID NOS: 30961, 30962, 30963, 30964, 30965, 30966, 30967, 30968, 30969, 30970, 30971 were selected from the ORFs having the nucleotide sequence represented by SEQ ID NO: 2264.

The probes having the nucleotide sequence represented by SEQ ID NOS: 30972, 30973, 30974, 30975, 30976, 30977, 30978, 30979, 30980, 30981, 30982 were selected from the ORFs having the nucleotide sequence represented by SEQ ID NO: 2265.

The probes having the nucleotide sequence represented by SEQ ID NOS: 30983, 30984, 30985, 30986, 30987, 30988, 30989, 30990, 30991, 30992, 30993 were selected from the ORFs having the nucleotide sequence represented by SEQ ID NO: 2266.

The probes having the nucleotide sequence represented by SEQ ID NOS: 30994, 30995, 30996, 30997, 30998, 30999, 31000, 31001, 31002, 31003, 31004 were selected from the ORFs having the nucleotide sequence represented by SEQ ID NO: 2267.

The probes having the nucleotide sequence represented by SEQ ID NOS: 31005, 31006, 31007, 31008, 31009, 31010, 31011, 31012, 31013, 31014, 31015 were selected from the ORFs having the nucleotide sequence represented by SEQ ID NO: 2268.

The probes having the nucleotide sequence represented by SEQ ID NOS: 31016, 31017, 31018, 31019, 31020, 31021, 31022, 31023, 31024, 31025, 31026 were selected from the ORFs having the nucleotide sequence represented by SEQ ID NO: 2269.

The probes having the nucleotide sequence represented by SEQ ID NOS: 31027, 31028, 31029, 31030, 31031, 31032, 31033, 31034, 31035, 31036, 31037 were selected from the ORFs having the nucleotide sequence represented by SEQ ID NO: 2270.

The probes having the nucleotide sequence represented by SEQ ID NOS: 31038, 31039, 31040, 31041, 31042, 31043, 31044, 31045, 31046, 31047, 31048, 31049, 31050, 31051, 31052, 31053, 31054, 31055, 31056, 31057, 31058, 31059 were selected from the ORFs having the nucleotide sequence represented by SEQ ID NO: 2271.

The probes having the nucleotide sequence represented by SEQ ID NOS: 31060, 31061, 31062, 31063, 31064, 31065, 31066, 31067, 31068, 31069, 31070 were selected from the ORFs having the nucleotide sequence represented by SEQ ID NO: 2272.

The probes having the nucleotide sequence represented by SEQ ID NOS: 31071, 31072, 31073, 31074, 31075, 31076, 31077, 31078, 31079, 31080, 31081 were selected from the ORFs having the nucleotide sequence represented by SEQ ID NO: 2273.

The probes having the nucleotide sequence represented by SEQ ID NOS: 31082, 31083, 31084, 31085, 31086, 31087, 31088, 31089, 31090, 31091, 31092 were selected from the ORFs having the nucleotide sequence represented by SEQ ID NO: 2274.

The probes having the nucleotide sequence represented by SEQ ID NOS: 31093, 31094, 31095, 31096, 31097, 31098, 31099, 31100, 31101, 31102, 31103 were selected from the ORFs having the nucleotide sequence represented by SEQ ID NO: 2275.

The probes having the nucleotide sequence represented by SEQ ID NOS: 31104, 31105, 31106, 31107, 31108, 31109, 31110, 31111, 31112, 31113, 31114 were selected from the ORFs having the nucleotide sequence represented by SEQ ID NO: 2276.

The probes having the nucleotide sequence represented by SEQ ID NOS: 31115, 31116, 31117, 31118, 31119, 31120, 31121, 31122, 31123, 31124, 31125 were selected from the ORFs having the nucleotide sequence represented by SEQ ID NO: 2277.

The probes having the nucleotide sequence represented by SEQ ID NOS: 31126, 31127, 31128, 31129, 31130, 31131, 31132, 31133, 31134, 31135, 31136 were selected from the ORFs having the nucleotide sequence represented by SEQ ID NO: 2278.

The probes having the nucleotide sequence represented by SEQ ID NOS: 31137, 31138, 31139, 31140, 31141, 31142, 31143, 31144, 31145, 31146, 31147 were selected from the ORFs having the nucleotide sequence represented by SEQ ID NO: 2279.

The probes having the nucleotide sequence represented by SEQ ID NOS: 31148, 31149, 31150, 31151, 31152, 31153, 31154, 31155, 31156, 31157, 31158 were selected from the ORFs having the nucleotide sequence represented by SEQ ID NO: 2280.

The probes having the nucleotide sequence represented by SEQ ID NOS: 31159, 31160, 31161, 31162, 31163, 31164, 31165, 31166, 31167, 31168, 31169 were selected from the ORFs having the nucleotide sequence represented by SEQ ID NO: 2281.

The probes having the nucleotide sequence represented by SEQ ID NOS: 31170, 31171, 31172, 31173, 31174, 31175, 31176, 31177, 31178, 31179, 31180 were selected from the ORFs having the nucleotide sequence represented by SEQ ID NO: 2282.

The probes having the nucleotide sequence represented by SEQ ID NOS: 31181, 31182, 31183, 31184, 31185, 31186, 31187, 31188, 31189, 31190, 31191 were selected from the ORFs having the nucleotide sequence represented by SEQ ID NO: 2283.

The probes having the nucleotide sequence represented by SEQ ID NOS: 31192, 31193, 31194, 31195, 31196, 31197, 31198, 31199, 31200, 31201, 31202 were selected from the ORFs having the nucleotide sequence represented by SEQ ID NO: 2284.

The probes having the nucleotide sequence represented by SEQ ID NOS: 31203, 31204, 31205, 31206, 31207, 31208, 31209, 31210, 31211, 31212, 31213 were selected from the ORFs having the nucleotide sequence represented by SEQ ID NO: 2285.

The probes having the nucleotide sequence represented by SEQ ID NOS: 31214, 31215, 31216, 31217, 31218, 31219, 31220, 31221, 31222, 31223, 31224 were selected from the ORFs having the nucleotide sequence represented by SEQ ID NO: 2286.

The probes having the nucleotide sequence represented by SEQ ID NOS: 31225, 31226, 31227, 31228, 31229, 31230, 31231, 31232, 31233, 31234, 31235 were selected from the ORFs having the nucleotide sequence represented by SEQ ID NO: 2287.

The probes having the nucleotide sequence represented by SEQ ID NOS: 31236, 31237, 31238, 31239, 31240, 31241, 31242, 31243, 31244, 31245, 31246 were selected from the ORFs having the nucleotide sequence represented by SEQ ID NO: 2288.

The probes having the nucleotide sequence represented by SEQ ID NOS: 31247, 31248, 31249, 31250, 31251, 31252, 31253, 31254, 31255, 31256, 31257 were selected from the ORFs having the nucleotide sequence represented by SEQ ID NO: 2289.

The probes having the nucleotide sequence represented by SEQ ID NOS: 31258, 31259, 31260, 31261, 31262, 31263, 31264, 31265, 31266, 31267, 31268 were selected from the ORFs having the nucleotide sequence represented by SEQ ID NO: 2290.

The probes having the nucleotide sequence represented by SEQ ID NOS: 31269, 31270, 31271, 31272, 31273, 31274, 31275, 31276, 31277, 31278, 31279 were selected from the ORFs having the nucleotide sequence represented by SEQ ID NO: 2291.

The probes having the nucleotide sequence represented by SEQ ID NOS: 31280, 31281, 31282, 31283, 31284, 31285, 31286, 31287, 31288, 31289, 31290 were selected from the ORFs having the nucleotide sequence represented by SEQ ID NO: 2292.

The probes having the nucleotide sequence represented by SEQ ID NOS: 31291, 31292, 31293, 31294, 31295, 31296, 31297, 31298, 31299, 31300, 31301 were selected from the ORFs having the nucleotide sequence represented by SEQ ID NO: 2293.

The probes having the nucleotide sequence represented by SEQ ID NOS: 31302, 31303, 31304, 31305, 31306, 31307, 31308, 31309, 31310, 31311, 31312 were selected from the ORFs having the nucleotide sequence represented by SEQ ID NO: 2294.

The probes having the nucleotide sequence represented by SEQ ID NOS: 31313, 31314, 31315, 31316, 31317, 31318, 31319, 31320, 31321, 31322, 31323 were selected from the ORFs having the nucleotide sequence represented by SEQ ID NO: 2295.

The probes having the nucleotide sequence represented by SEQ ID NOS: 31324, 31325, 31326, 31327, 31328, 31329, 31330, 31331, 31332, 31333, 31334 were selected from the ORFs having the nucleotide sequence represented by SEQ ID NO: 2296.

The probes having the nucleotide sequence represented by SEQ ID NOS: 31335, 31336, 31337, 31338, 31339, 31340, 31341, 31342, 31343, 31344, 31345 were selected from the ORFs having the nucleotide sequence represented by SEQ ID NO: 2297.

The probes having the nucleotide sequence represented by SEQ ID NOS: 31346, 31347, 31348, 31349, 31350, 31351, 31352, 31353, 31354, 31355, 31356 were selected from the ORFs having the nucleotide sequence represented by SEQ ID NO: 2298.

The probes having the nucleotide sequence represented by SEQ ID NOS: 31357, 31358, 31359, 31360, 31361, 31362, 31363, 31364, 31365, 31366, 31367 were selected from the ORFs having the nucleotide sequence represented by SEQ ID NO: 2299.

The probes having the nucleotide sequence represented by SEQ ID NOS: 31368, 31369, 31370, 31371, 31372, 31373, 31374, 31375, 31376, 31377, 31378 were selected from the ORFs having the nucleotide sequence represented by SEQ ID NO: 2300.

The probes having the nucleotide sequence represented by SEQ ID NOS: 31379, 31380, 31381, 31382, 31383, 31384, 31385, 31386, 31387, 31388, 31389 were selected from the ORFs having the nucleotide sequence represented by SEQ ID NO: 2301.

The probes having the nucleotide sequence represented by SEQ ID NOS: 31390, 31391, 31392, 31393, 31394, 31395, 31396, 31397, 31398, 31399, 31400 were selected from the ORFs having the nucleotide sequence represented by SEQ ID NO: 2302.

The probes having the nucleotide sequence represented by SEQ ID NOS: 31401, 31402, 31403, 31404, 31405, 31406, 31407, 31408, 31409, 31410, 31411 were selected from the ORFs having the nucleotide sequence represented by SEQ ID NO: 2303.

The probes having the nucleotide sequence represented by SEQ ID NOS: 31412, 31413, 31414, 31415, 31416, 31417, 31418, 31419, 31420, 31421, 31422 were selected from the ORFs having the nucleotide sequence represented by SEQ ID NO: 2304.

The probes having the nucleotide sequence represented by SEQ ID NOS: 31423, 31424, 31425, 31426, 31427, 31428, 31429, 31430, 31431, 31432, 31433 were selected from the ORFs having the nucleotide sequence represented by SEQ ID NO: 2305.

The probes having the nucleotide sequence represented by SEQ ID NOS: 31434, 31435, 31436, 31437, 31438, 31439, 31440, 31441, 31442, 31443, 31444 were selected from the ORFs having the nucleotide sequence represented by SEQ ID NO: 2306.

The probes having the nucleotide sequence represented by SEQ ID NOS: 31445, 31446, 31447, 31448, 31449, 31450, 31451, 31452, 31453, 31454, 31455 were selected from the ORFs having the nucleotide sequence represented by SEQ ID NO: 2307.

The probes having the nucleotide sequence represented by SEQ ID NOS: 31456, 31457, 31458, 31459, 31460, 31461, 31462, 31463, 31464, 31465, 31466 were selected from the ORFs having the nucleotide sequence represented by SEQ ID NO: 2308.

The probes having the nucleotide sequence represented by SEQ ID NOS: 31467, 31468, 31469, 31470, 31471, 31472, 31473, 31474, 31475, 31476, 31477 were selected from the ORFs having the nucleotide sequence represented by SEQ ID NO: 2309.

The probes having the nucleotide sequence represented by SEQ ID NOS: 31478, 31479, 31480, 31481, 31482, 31483, 31484, 31485, 31486, 31487, 31488 were selected from the ORFs having the nucleotide sequence represented by SEQ ID NO: 2310.

The probes having the nucleotide sequence represented by SEQ ID NOS: 31489, 31490, 31491, 31492, 31493, 31494, 31495, 31496, 31497, 31498, 31499 were selected from the ORFs having the nucleotide sequence represented by SEQ ID NO: 2311.

The probes having the nucleotide sequence represented by SEQ ID NOS: 31500, 31501, 31502, 31503, 31504, 31505, 31506, 31507, 31508, 31509, 31510 were selected from the ORFs having the nucleotide sequence represented by SEQ ID NO: 2312.

The probes having the nucleotide sequence represented by SEQ ID NOS: 31511, 31512, 31513, 31514, 31515, 31516, 31517, 31518, 31519, 31520, 31521 were selected from the ORFs having the nucleotide sequence represented by SEQ ID NO: 2313.

The probes having the nucleotide sequence represented by SEQ ID NOS: 31522, 31523, 31524, 31525, 31526, 31527, 31528, 31529, 31530, 31531, 31532 were selected from the ORFs having the nucleotide sequence represented by SEQ ID NO: 2314.

The probes having the nucleotide sequence represented by SEQ ID NOS: 31533, 31534, 31535, 31536, 31537, 31538, 31539, 31540, 31541, 31542, 31543 were selected from the ORFs having the nucleotide sequence represented by SEQ ID NO: 2315.

The probes having the nucleotide sequence represented by SEQ ID NOS: 31544, 31545, 31546, 31547, 31548, 31549, 31550, 31551, 31552, 31553, 31554 were selected from the ORFs having the nucleotide sequence represented by SEQ ID NO: 2316.

The probes having the nucleotide sequence represented by SEQ ID NOS: 31555, 31556, 31557, 31558, 31559, 31560, 31561, 31562, 31563, 31564, 31565 were selected from the ORFs having the nucleotide sequence represented by SEQ ID NO: 2317.

The probes having the nucleotide sequence represented by SEQ ID NOS: 31566, 31567, 31568, 31569, 31570, 31571, 31572, 31573, 31574, 31575, 31576 were selected from the ORFs having the nucleotide sequence represented by SEQ ID NO: 2318.

The probes having the nucleotide sequence represented by SEQ ID NOS: 31577, 31578, 31579, 31580, 31581, 31582, 31583, 31584, 31585, 31586, 31587 were selected from the ORFs having the nucleotide sequence represented by SEQ ID NO: 2319.

The probes having the nucleotide sequence represented by SEQ ID NOS: 31588, 31589, 31590, 31591, 31592, 31593, 31594, 31595, 31596, 31597, 31598 were selected from the ORFs having the nucleotide sequence represented by SEQ ID NO: 2320.

The probes having the nucleotide sequence represented by SEQ ID NOS: 31599, 31600, 31601, 31602, 31603, 31604, 31605, 31606, 31607, 31608, 31609 were selected from the ORFs having the nucleotide sequence represented by SEQ ID NO: 2321.

The probes having the nucleotide sequence represented by SEQ ID NOS: 31610, 31611, 31612, 31613, 31614, 31615, 31616, 31617, 31618, 31619, 31620 were selected from the ORFs having the nucleotide sequence represented by SEQ ID NO: 2322.

The probes having the nucleotide sequence represented by SEQ ID NOS: 31621, 31622, 31623, 31624, 31625, 31626, 31627, 31628, 31629, 31630, 31631 were selected from the ORFs having the nucleotide sequence represented by SEQ ID NO: 2323.

The probes having the nucleotide sequence represented by SEQ ID NOS: 31632, 31633, 31634, 31635, 31636, 31637, 31638, 31639, 31640, 31641, 31642 were selected from the ORFs having the nucleotide sequence represented by SEQ ID NO: 2324.

The probes having the nucleotide sequence represented by SEQ ID NOS: 31643, 31644, 31645, 31646, 31647, 31648, 31649, 31650, 31651, 31652, 31653 were selected from the ORFs having the nucleotide sequence represented by SEQ ID NO: 2325.

The probes having the nucleotide sequence represented by SEQ ID NOS: 31654, 31655, 31656, 31657, 31658, 31659, 31660, 31661, 31662, 31663, 31664 were selected from the ORFs having the nucleotide sequence represented by SEQ ID NO: 2326.

The probes having the nucleotide sequence represented by SEQ ID NOS: 31665, 31666, 31667, 31668, 31669, 31670, 31671, 31672, 31673, 31674, 31675 were selected from the ORFs having the nucleotide sequence represented by SEQ ID NO: 2327.

The probes having the nucleotide sequence represented by SEQ ID NOS: 31676, 31677, 31678, 31679, 31680, 31681, 31682, 31683, 31684, 31685, 31686 were selected from the ORFs having the nucleotide sequence represented by SEQ ID NO: 2328.

The probes having the nucleotide sequence represented by SEQ ID NOS: 31687, 31688, 31689, 31690, 31691, 31692, 31693, 31694, 31695, 31696, 31697 were selected from the ORFs having the nucleotide sequence represented by SEQ ID NO: 2329.

The probes having the nucleotide sequence represented by SEQ ID NOS: 31698, 31699, 31700, 31701, 31702, 31703, 31704, 31705, 31706, 31707, 31708 were selected from the ORFs having the nucleotide sequence represented by SEQ ID NO: 2330.

The probes having the nucleotide sequence represented by SEQ ID NOS: 31709, 31710, 31711, 31712, 31713, 31714, 31715, 31716, 31717, 31718, 31719, 31720, 31721, 31722, 31723, 31724, 31725, 31726, 31727, 31728, 31729, 31730 were selected from the ORFs having the nucleotide sequence represented by SEQ ID NO: 2331.

The probes having the nucleotide sequence represented by SEQ ID NOS: 31731, 31732, 31733, 31734, 31735, 31736, 31737, 31738, 31739, 31740, 31741 were selected from the ORFs having the nucleotide sequence represented by SEQ ID NO: 2332.

The probes having the nucleotide sequence represented by SEQ ID NOS: 31742, 31743, 31744, 31745, 31746, 31747, 31748, 31749, 31750, 31751, 31752 were selected from the ORFs having the nucleotide sequence represented by SEQ ID NO: 2333.

The probes having the nucleotide sequence represented by SEQ ID NOS: 31753, 31754, 31755, 31756, 31757, 31758, 31759, 31760, 31761, 31762, 31763 were selected from the ORFs having the nucleotide sequence represented by SEQ ID NO: 2334.

The probes having the nucleotide sequence represented by SEQ ID NOS: 31764, 31765, 31766, 31767, 31768, 31769, 31770, 31771, 31772, 31773, 31774 were selected from the ORFs having the nucleotide sequence represented by SEQ ID NO: 2335.

The probes having the nucleotide sequence represented by SEQ ID NOS: 31775, 31776, 31777, 31778, 31779, 31780, 31781, 31782, 31783, 31784, 31785 were selected from the ORFs having the nucleotide sequence represented by SEQ ID NO: 2336.

The probes having the nucleotide sequence represented by SEQ ID NOS: 31786, 31787, 31788, 31789, 31790, 31791, 31792, 31793, 31794, 31795, 31796 were selected from the ORFs having the nucleotide sequence represented by SEQ ID NO: 2337.

The probes having the nucleotide sequence represented by SEQ ID NOS: 31797, 31798, 31799, 31800, 31801, 31802, 31803, 31804, 31805, 31806, 31807 were selected from the ORFs having the nucleotide sequence represented by SEQ ID NO: 2338.

The probes having the nucleotide sequence represented by SEQ ID NOS: 31808, 31809, 31810, 31811, 31812, 31813, 31814, 31815, 31816, 31817, 31818 were selected from the ORFs having the nucleotide sequence represented by SEQ ID NO: 2339.

The probes having the nucleotide sequence represented by SEQ ID NOS: 31819, 31820, 31821, 31822, 31823, 31824, 31825, 31826, 31827, 31828, 31829 were selected from the ORFs having the nucleotide sequence represented by SEQ ID NO: 2340.

The probes having the nucleotide sequence represented by SEQ ID NOS: 31830, 31831, 31832, 31833, 31834, 31835, 31836, 31837, 31838, 31839, 31840 were selected from the ORFs having the nucleotide sequence represented by SEQ ID NO: 2341.

The probes having the nucleotide sequence represented by SEQ ID NOS: 31841, 31842, 31843, 31844, 31845, 31846, 31847, 31848, 31849, 31850, 31851 were selected from the ORFs having the nucleotide sequence represented by SEQ ID NO: 2342.

The probes having the nucleotide sequence represented by SEQ ID NOS: 31852, 31.853, 31854, 31855, 31856, 31857, 31858, 31859, 31860, 31861, 31862 were selected from the ORFs having the nucleotide sequence represented by SEQ ID NO: 2343.

The probes having the nucleotide sequence represented by SEQ ID NOS: 31863, 31864, 31865, 31866, 31867, 31868, 31869, 31870, 31871, 31872, 31873 were selected from the ORFs having the nucleotide sequence represented by SEQ ID NO: 2344.

The probes having the nucleotide sequence represented by SEQ ID NOS: 31874, 31875, 31876, 31877, 31878, 31879, 31880, 31881, 31882, 31883, 31884 were selected from the ORFs having the nucleotide sequence represented by SEQ ID NO: 2345.

The probes having the nucleotide sequence represented by SEQ ID NOS: 31885, 31886, 31887, 31888, 31889, 31890, 31891, 31892, 31893, 31894, 31895 were selected from the ORFs having the nucleotide sequence represented by SEQ ID NO: 2346.

The probes having the nucleotide sequence represented by SEQ ID NOS: 31896, 31897, 31898, 31899, 31900, 31901, 31902, 31903, 31904, 31905, 31906 were selected from the ORFs having the nucleotide sequence represented by SEQ ID NO: 2347.

The probes having the nucleotide sequence represented by SEQ ID NOS: 31907, 31908, 31909, 31910, 31911, 31912, 31913, 31914, 31915, 31916, 31917 were selected from the ORFs having the nucleotide sequence represented by SEQ ID NO: 2348.

The probes having the nucleotide sequence represented by SEQ ID NOS: 31918, 31919, 31920, 31921, 31922, 31923, 31924, 31925, 31926, 31927, 31928 were selected from the ORFs having the nucleotide sequence represented by SEQ ID NO: 2349.

The probes having the nucleotide sequence represented by SEQ ID NOS: 31929, 31930, 31931, 31932, 31933, 31934, 31935, 31936, 31937, 31938, 31939 were selected from the ORFs having the nucleotide sequence represented by SEQ ID NO: 2350.

The probes having the nucleotide sequence represented by SEQ ID NOS: 31940, 31941, 31942, 31943, 31944, 31945, 31946, 31947, 31948, 31949, 31950 were selected from the ORFs having the nucleotide sequence represented by SEQ ID NO: 2351.

The probes having the nucleotide sequence represented by SEQ ID NOS: 31951, 31952, 31953, 31954, 31955, 31956, 31957, 31958, 31959, 31960, 31961 were selected from the ORFs having the nucleotide sequence represented by SEQ ID NO: 2352.

The probes having the nucleotide sequence represented by SEQ ID NOS: 31962, 31963, 31964, 31965, 31966, 31967, 31968, 31969, 31970, 31971, 31972 were selected from the ORFs having the nucleotide sequence represented by SEQ ID NO: 2353.

The probes having the nucleotide sequence represented by SEQ ID NOS: 31973, 31974, 31975, 31976, 31977, 31978, 31979, 31980, 31981, 31982, 31983 were selected from the ORFs having the nucleotide sequence represented by SEQ ID NO: 2354.

The probes having the nucleotide sequence represented by SEQ ID NOS: 31984, 31985, 31986, 31987, 31988, 31989, 31990, 31991, 31992, 31993, 31994 were selected from the ORFs having the nucleotide sequence represented by SEQ ID NO: 2355.

The probes having the nucleotide sequence represented by SEQ ID NOS: 31995, 31996, 31997, 31998, 31999, 32000, 32001, 32002, 32003, 32004, 32005 were selected from the ORFs having the nucleotide sequence represented by SEQ ID NO: 2356.

The probes having the nucleotide sequence represented by SEQ ID NOS: 32006, 32007, 32008, 32009, 32010, 32011, 32012, 32013, 32014, 32015, 32016 were selected from the ORFs having the nucleotide sequence represented by SEQ ID NO: 2357.

The probes having the nucleotide sequence represented by SEQ ID NOS: 32017, 32018, 32019, 32020, 32021, 32022, 32023, 32024, 32025, 32026, 32027 were selected from the ORFs having the nucleotide sequence represented by SEQ ID NO: 2358.

The probes having the nucleotide sequence represented by SEQ ID NOS: 32028, 32029, 32030, 32031, 32032, 32033, 32034, 32035, 32036, 32037, 32038 were selected from the ORFs having the nucleotide sequence represented by SEQ ID NO: 2359.

The probes having the nucleotide sequence represented by SEQ ID NOS: 32039, 32040, 32041, 32042, 32043, 32044, 32045, 32046, 32047, 32048, 32049 were selected from the ORFs having the nucleotide sequence represented by SEQ ID NO: 2360.

The probes having the nucleotide sequence represented by SEQ ID NOS: 32050, 32051, 32052, 32053, 32054, 32055, 32056, 32057, 32058, 32059, 32060 were selected from the ORFs having the nucleotide sequence represented by SEQ ID NO: 2361.

The probes having the nucleotide sequence represented by SEQ ID NOS: 32061, 32062, 32063, 32064, 32065, 32066, 32067, 32068, 32069, 32070, 32071 were selected from the ORFs having the nucleotide sequence represented by SEQ ID NO: 2362.

The probes having the nucleotide sequence represented by SEQ ID NOS: 32072, 32073, 32074, 32075, 32076, 32077, 32078, 32079, 32080, 32081, 32082 were selected from the ORFs having the nucleotide sequence represented by SEQ ID NO: 2363.

The probes having the nucleotide sequence represented by SEQ ID NOS: 32083, 32084, 32085, 32086, 32087, 32088, 32089, 32090, 32091, 32092, 32093 were selected from the ORFs having the nucleotide sequence represented by SEQ ID NO: 2364.

The probes having the nucleotide sequence represented by SEQ ID NOS: 32094, 32095, 32096, 32097, 32098, 32099, 32100, 32101, 32102, 32103, 32104 were selected from the ORFs having the nucleotide sequence represented by SEQ ID NO: 2365.

The probes having the nucleotide sequence represented by SEQ ID NOS: 32105, 32106, 32107, 32108, 32109, 32110, 32111, 32112, 32113, 32114, 32115 were selected from the ORFs having the nucleotide sequence represented by SEQ ID NO: 2366.

The probes having the nucleotide sequence represented by SEQ ID NOS: 32116, 32117, 32118, 32119, 32120, 32121, 32122, 32123, 32124, 32125, 32126 were selected from the ORFs having the nucleotide sequence represented by SEQ ID NO: 2367.

The probes having the nucleotide sequence represented by SEQ ID NOS: 32127, 32128, 32129, 32130, 32131, 32132, 32133, 32134, 32135, 32136, 32137 were selected from the ORFs having the nucleotide sequence represented by SEQ ID NO: 2368.

The probes having the nucleotide sequence represented by SEQ ID NOS: 32138, 32139, 32140, 32141, 32142, 32143, 32144, 32145, 32146, 32147, 32148 were selected from the ORFs having the nucleotide sequence represented by SEQ ID NO: 2369.

The probes having the nucleotide sequence represented by SEQ ID NOS: 32149, 32150, 32151, 32152, 32153, 32154, 32155, 32156, 32157, 32158, 32159 were selected from the ORFs having the nucleotide sequence represented by SEQ ID NO: 2370.

The probes having the nucleotide sequence represented by SEQ ID NOS: 32160, 32161, 32162, 32163, 32164, 32165, 32166, 32167, 32168, 32169, 32170 were selected from the ORFs having the nucleotide sequence represented by SEQ ID NO: 2371.

The probes having the nucleotide sequence represented by SEQ ID NOS: 32171, 32172, 32173, 32174, 32175, 32176, 32177, 32178, 32179, 32180, 32181 were selected from the ORFs having the nucleotide sequence represented by SEQ ID NO: 2372.

The probes having the nucleotide sequence represented by SEQ ID NOS: 32182, 32183, 32184, 32185, 32186, 32187, 32188, 32189, 32190, 32191, 32192 were selected from the ORFs having the nucleotide sequence represented by SEQ ID NO: 2373.

The probes having the nucleotide sequence represented by SEQ ID NOS: 32193, 32194, 32195, 32196, 32197, 32198, 32199, 32200, 32201, 32202, 32203 were selected from the ORFs having the nucleotide sequence represented by SEQ ID NO: 2374.

The probes having the nucleotide sequence represented by SEQ ID NOS: 32204, 32205, 32206, 32207, 32208, 32209, 32210, 32211, 32212, 32213, 32214 were selected from the ORFs having the nucleotide sequence represented by SEQ ID NO: 2375.

The probes having the nucleotide sequence represented by SEQ ID NOS: 32215, 32216, 32217, 32218, 32219, 32220, 32221, 32222, 32223, 32224, 32225 were selected from the ORFs having the nucleotide sequence represented by SEQ ID NO: 2376.

The probes having the nucleotide sequence represented by SEQ ID NOS: 32226, 32227, 32228, 32229, 32230, 32231, 32232, 32233, 32234, 32235, 32236 were selected from the ORFs having the nucleotide sequence represented by SEQ ID NO: 2377.

The probes having the nucleotide sequence represented by SEQ ID NOS: 32237, 32238, 32239, 32240, 32241, 32242, 32243, 32244, 32245, 32246, 32247 were selected from the ORFs having the nucleotide sequence represented by SEQ ID NO: 2378.

The probes having the nucleotide sequence represented by SEQ ID NOS: 32248, 32249, 32250, 32251, 32252, 32253, 32254, 32255, 32256, 32257, 32258 were selected from the ORFs having the nucleotide sequence represented by SEQ ID NO: 2379.

The probes having the nucleotide sequence represented by SEQ ID NOS: 32259, 32260, 32261, 32262, 32263, 32264, 32265, 32266, 32267, 32268, 32269 were selected from the ORFs having the nucleotide sequence represented by SEQ ID NO: 2380.

The probes having the nucleotide sequence represented by SEQ ID NOS: 32270, 32271, 32272, 32273, 32274, 32275, 32276, 32277, 32278, 32279, 32280 were selected from the ORFs having the nucleotide sequence represented by SEQ ID NO: 2381.

The probes having the nucleotide sequence represented by SEQ ID NOS: 32281, 32282, 32283, 32284, 32285, 32286, 32287, 32288, 32289, 32290, 32291 were selected from the ORFs having the nucleotide sequence represented by SEQ ID NO: 2382.

The probes having the nucleotide sequence represented by SEQ ID NOS: 32292, 32293, 32294, 32295, 32296, 32297, 32298, 32299, 32300, 32301, 32302 were selected from the ORFs having the nucleotide sequence represented by SEQ ID NO: 2383.

The probes having the nucleotide sequence represented by SEQ ID NOS: 32303, 32304, 32305, 32306, 32307, 32308, 32309, 32310, 32311, 32312, 32313 were selected from the ORFs having the nucleotide sequence represented by SEQ ID NO: 2384.

The probes having the nucleotide sequence represented by SEQ ID NOS: 32314, 32315, 32316, 32317, 32318, 32319, 32320, 32321, 32322, 32323, 32324 were selected from the ORFs having the nucleotide sequence represented by SEQ ID NO: 2385.

The probes having the nucleotide sequence represented by SEQ ID NOS: 32325, 32326, 32327, 32328, 32329, 32330, 32331, 32332, 32333, 32334, 32335 were selected from the ORFs having the nucleotide sequence represented by SEQ ID NO: 2386.

The probes having the nucleotide sequence represented by SEQ ID NOS: 32336, 32337, 32338, 32339, 32340, 32341, 32342, 32343, 32344, 32345, 32346 were selected from the ORFs having the nucleotide sequence represented by SEQ ID NO: 2387.

The probes having the nucleotide sequence represented by SEQ ID NOS: 32347, 32348, 32349, 32350, 32351, 32352, 32353, 32354, 32355, 32356, 32357 were selected from the ORFs having the nucleotide sequence represented by SEQ ID NO: 2388.

The probes having the nucleotide sequence represented by SEQ ID NOS: 32358, 32359, 32360, 32361, 32362, 32363, 32364, 32365, 32366, 32367, 32368 were selected from the ORFs having the nucleotide sequence represented by SEQ ID NO: 2389.

The probes having the nucleotide sequence represented by SEQ ID NOS: 32369, 32370, 32371, 32372, 32373, 32374, 32375, 32376, 32377, 32378, 32379 were selected from the ORFs having the nucleotide sequence represented by SEQ ID NO: 2390.

The probes having the nucleotide sequence represented by SEQ ID NOS: 32380, 32381, 32382, 32383, 32384, 32385, 32386, 32387, 32388, 32389, 32390 were selected from the ORFs having the nucleotide sequence represented by SEQ ID NO: 2391.

The probes having the nucleotide sequence represented by SEQ ID NOS: 32391, 32392, 32393, 32394, 32395, 32396, 32397, 32398, 32399, 32400, 32401 were selected from the ORFs having the nucleotide sequence represented by SEQ ID NO: 2392.

The probes having the nucleotide sequence represented by SEQ ID NOS: 32402, 32403, 32404, 32405, 32406, 32407, 32408, 32409, 32410, 32411, 32412 were selected from the ORFs having the nucleotide sequence represented by SEQ ID NO: 2393.

The probes having the nucleotide sequence represented by SEQ ID NOS: 32413, 32414, 32415, 32416, 32417, 32418, 32419, 32420, 32421, 32422, 32423 were selected from the ORFs having the nucleotide sequence represented by SEQ ID NO: 2394.

The probes having the nucleotide sequence represented by SEQ ID NOS: 32424, 32425, 32426, 32427, 32428, 32429, 32430, 32431, 32432, 32433, 32434 were selected from the ORFs having the nucleotide sequence represented by SEQ ID NO: 2395.

The probes having the nucleotide sequence represented by SEQ ID NOS: 32435, 32436, 32437, 32438, 32439, 32440, 32441, 32442, 32443, 32444, 32445 were selected from the ORFs having the nucleotide sequence represented by SEQ ID NO: 2396.

The probes having the nucleotide sequence represented by SEQ ID NOS: 32446, 32447, 32448, 32449, 32450, 32451, 32452, 32453, 32454, 32455, 32456 were selected from the ORFs having the nucleotide sequence represented by SEQ ID NO: 2397.

The probes having the nucleotide sequence represented by SEQ ID NOS: 32457, 32458, 32459, 32460, 32461, 32462, 32463, 32464, 32465, 32466, 32467 were selected from the ORFs having the nucleotide sequence represented by SEQ ID NO: 2398.

The probes having the nucleotide sequence represented by SEQ ID NOS: 32468, 32469, 32470, 32471, 32472, 32473, 32474, 32475, 32476, 32477, 32478 were selected from the ORFs having the nucleotide sequence represented by SEQ ID NO: 2399.

The probes having the nucleotide sequence represented by SEQ ID NOS: 32479, 32480, 32481, 32482, 32483, 32484, 32485, 32486, 32487, 32488, 32489 were selected from the ORFs having the nucleotide sequence represented by SEQ ID NO: 2400.

The probes having the nucleotide sequence represented by SEQ ID NOS: 32490, 32491, 32492, 32493, 32494, 32495, 32496, 32497, 32498, 32499, 32500 were selected from the ORFs having the nucleotide sequence represented by SEQ ID NO: 2401.

The probes having the nucleotide sequence represented by SEQ ID NOS: 32501, 32502, 32503, 32504, 32505, 32506, 32507, 32508, 32509, 32510, 32511 were selected from the ORFs having the nucleotide sequence represented by SEQ ID NO: 2402.

The probes having the nucleotide sequence represented by SEQ ID NOS: 32512, 32513, 32514, 32515, 32516, 32517, 32518, 32519, 32520, 32521, 32522 were selected from the ORFs having the nucleotide sequence represented by SEQ ID NO: 2403.

The probes having the nucleotide sequence represented by SEQ ID NOS: 32523, 32524, 32525, 32526, 32527, 32528, 32529, 32530, 32531, 32532, 32533 were selected from the ORFs having the nucleotide sequence represented by SEQ ID NO: 2404.

The probes having the nucleotide sequence represented by SEQ ID NOS: 32534, 32535, 32536, 32537, 32538, 32539, 32540, 32541, 32542, 32543, 32544 were selected from the ORFs having the nucleotide sequence represented by SEQ ID NO: 2405.

The probes having the nucleotide sequence represented by SEQ ID NOS: 32545, 32546, 32547, 32548, 32549, 32550, 32551, 32552, 32553, 32554, 32555 were selected from the ORFs having the nucleotide sequence represented by SEQ ID NO: 2406.

The probes having the nucleotide sequence represented by SEQ ID NOS: 32556, 32557, 32558, 32559, 32560, 32561, 32562, 32563, 32564, 32565, 32566 were selected from the ORFs having the nucleotide sequence represented by SEQ ID NO: 2407.

The probes having the nucleotide sequence represented by SEQ ID NOS: 32567, 32568, 32569, 32570, 32571, 32572, 32573, 32574, 32575, 32576, 32577 were selected from the ORFs having the nucleotide sequence represented by SEQ ID NO: 2408.

The probes having the nucleotide sequence represented by SEQ ID NOS: 32578, 32579, 32580, 32581, 32582, 32583, 32584, 32585, 32586, 32587, 32588 were selected from the ORFs having the nucleotide sequence represented by SEQ ID NO: 2409.

The probes having the nucleotide sequence represented by SEQ ID NOS: 32589, 32590, 32591, 32592, 32593, 32594, 32595, 32596, 32597, 32598, 32599 were selected from the ORFs having the nucleotide sequence represented by SEQ ID NO: 2410.

The probes having the nucleotide sequence represented by SEQ ID NOS: 32600, 32601, 32602, 32603, 32604, 32605, 32606, 32607, 32608, 32609, 32610 were selected from the ORFs having the nucleotide sequence represented by SEQ ID NO: 2411.

The probes having the nucleotide sequence represented by SEQ ID NOS: 32611, 32612, 32613, 32614, 32615, 32616, 32617, 32618, 32619, 32620, 32621 were selected from the ORFs having the nucleotide sequence represented by SEQ ID NO: 2412.

The probes having the nucleotide sequence represented by SEQ ID NOS: 32622, 32623, 32624, 32625, 32626, 32627, 32628, 32629, 32630, 32631, 32632 were selected from the ORFs having the nucleotide sequence represented by SEQ ID NO: 2413.

The probes having the nucleotide sequence represented by SEQ ID NOS: 32633, 32634, 32635, 32636, 32637, 32638, 32639, 32640, 32641, 32642, 32643 were selected from the ORFs having the nucleotide sequence represented by SEQ ID NO: 2414.

The probes having the nucleotide sequence represented by SEQ ID NOS: 32644, 32645, 32646, 32647, 32648, 32649, 32650, 32651, 32652, 32653, 32654 were selected from the ORFs having the nucleotide sequence represented by SEQ ID NO: 2415.

The probes having the nucleotide sequence represented by SEQ ID NOS: 32655, 32656, 32657, 32658, 32659, 32660, 32661, 32662, 32663, 32664, 32665 were selected from the ORFs having the nucleotide sequence represented by SEQ ID NO: 2416.

The probes having the nucleotide sequence represented by SEQ ID NOS: 32666, 32667, 32668, 32669, 32670, 32671, 32672, 32673, 32674, 32675, 32676 were selected from the ORFs having the nucleotide sequence represented by SEQ ID NO: 2417.

The probes having the nucleotide sequence represented by SEQ ID NOS: 32677, 32678, 32679, 32680, 32681, 32682, 32683, 32684, 32685, 32686, 32687 were selected from the ORFs having the nucleotide sequence represented by SEQ ID NO: 2418.

The probes having the nucleotide sequence represented by SEQ ID NOS: 32688, 32689, 32690, 32691, 32692, 32693, 32694, 32695, 32696, 32697, 32698 were selected from the ORFs having the nucleotide sequence represented by SEQ ID NO: 2419.

The probes having the nucleotide sequence represented by SEQ ID NOS: 32699, 32700, 32701, 32702, 32703, 32704, 32705, 32706, 32707, 32708, 32709 were selected from the ORFs having the nucleotide sequence represented by SEQ ID NO: 2420.

The probes having the nucleotide sequence represented by SEQ ID NOS: 32710, 32711, 32712, 32713, 32714, 32715, 32716, 32717, 32718, 32719, 32720 were selected from the ORFs having the nucleotide sequence represented by SEQ ID NO: 2421.

The probes having the nucleotide sequence represented by SEQ ID NOS: 32721, 32722, 32723, 32724, 32725, 32726, 32727, 32728, 32729, 32730, 32731 were selected from the ORFs having the nucleotide sequence represented by SEQ ID NO: 2422.

The probes having the nucleotide sequence represented by SEQ ID NOS: 32732, 32733, 32734, 32735, 32736, 32737, 32738, 32739, 32740, 32741, 32742 were selected from the ORFs having the nucleotide sequence represented by SEQ ID NO: 2423.

The probes having the nucleotide sequence represented by SEQ ID NOS: 32743, 32744, 32745, 32746, 32747, 32748, 32749, 32750, 32751 were selected from the ORFs having the nucleotide sequence represented by SEQ ID NO: 2424.

The probes having the nucleotide sequence represented by SEQ ID NOS: 32752, 32753, 32754, 32755, 32756, 32757, 32758, 32759, 32760, 32761, 32762 were selected from the ORFs having the nucleotide sequence represented by SEQ ID NO: 2425.

The probes having the nucleotide sequence represented by SEQ ID NOS: 32763, 32764, 32765, 32766, 32767, 32768, 32769, 32770, 32771, 32772, 32773 were selected from the ORFs having the nucleotide sequence represented by SEQ ID NO: 2426.

The probes having the nucleotide sequence represented by SEQ ID NOS: 32774, 32775, 32776, 32777, 32778, 32779, 32780, 32781, 32782, 32783, 32784 were selected from the ORFs having the nucleotide sequence represented by SEQ ID NO: 2427.

The probes having the nucleotide sequence represented by SEQ ID NOS: 32785, 32786, 32787, 32788, 32789, 32790, 32791, 32792, 32793, 32794, 32795 were selected from the ORFs having the nucleotide sequence represented by SEQ ID NO: 2428.

The probes having the nucleotide sequence represented by SEQ ID NOS: 32796, 32797, 32798, 32799, 32800, 32801, 32802, 32803, 32804, 32805, 32806 were selected from the ORFs having the nucleotide sequence represented by SEQ ID NO: 2429.

The probes having the nucleotide sequence represented by SEQ ID NOS: 32807, 32808, 32809, 32810, 32811, 32812, 32813, 32814, 32815, 32816, 32817 were selected from the ORFs having the nucleotide sequence represented by SEQ ID NO: 2430.

The probes having the nucleotide sequence represented by SEQ ID NOS: 32818, 32819, 32820, 32821, 32822, 32823, 32824, 32825, 32826, 32827, 32828 were selected from the ORFs having the nucleotide sequence represented by SEQ ID NO: 2431.

The probes having the nucleotide sequence represented by SEQ ID NOS: 32829, 32830, 32831, 32832, 32833, 32834, 32835, 32836, 32837, 32838, 32839 were selected from the ORFs having the nucleotide sequence represented by SEQ ID NO: 2432.

The probes having the nucleotide sequence represented by SEQ ID NOS: 32840, 32841, 32842, 32843, 32844, 32845, 32846, 32847, 32848, 32849, 32850 were selected from the ORFs having the nucleotide sequence represented by SEQ ID NO: 2433.

The probes having the nucleotide sequence represented by SEQ ID NOS: 32851, 32852, 32853, 32854, 32855, 32856, 32857, 32858, 32859, 32860, 32861 were selected from the ORFs having the nucleotide sequence represented by SEQ ID NO: 2434.

The probes having the nucleotide sequence represented by SEQ ID NOS: 32862, 32863, 32864, 32865, 32866, 32867, 32868, 32869, 32870, 32871, 32872 were selected from the ORFs having the nucleotide sequence represented by SEQ ID NO: 2435.

The probes having the nucleotide sequence represented by SEQ ID NOS: 32873, 32874, 32875, 32876, 32877, 32878, 32879, 32880, 32881, 32882, 32883 were selected from the ORFs having the nucleotide sequence represented by SEQ ID NO: 2436.

The probes having the nucleotide sequence represented by SEQ ID NOS: 32884, 32885, 32886, 32887, 32888, 32889, 32890, 32891, 32892, 32893, 32894 were selected from the ORFs having the nucleotide sequence represented by SEQ ID NO: 2437.

The probes having the nucleotide sequence represented by SEQ ID NOS: 32895, 32896, 32897, 32898, 32899, 32900, 32901, 32902, 32903, 32904, 32905 were selected from the ORFs having the nucleotide sequence represented by SEQ ID NO: 2438.

The probes having the nucleotide sequence represented by SEQ ID NOS: 32906, 32907, 32908, 32909, 32910, 32911, 32912, 32913, 32914, 32915, 32916 were selected from the ORFs having the nucleotide sequence represented by SEQ ID NO: 2439.

The probes having the nucleotide sequence represented by SEQ ID NOS: 32917, 32918, 32919, 32920, 32921, 32922, 32923, 32924, 32925, 32926, 32927 were selected from the ORFs having the nucleotide sequence represented by SEQ ID NO: 2440.

The probes having the nucleotide sequence represented by SEQ ID NOS: 32928, 32929, 32930, 32931, 32932, 32933, 32934, 32935, 32936, 32937, 32938 were selected from the ORFs having the nucleotide sequence represented by SEQ ID NO: 2441.

The probes having the nucleotide sequence represented by SEQ ID NOS: 32939, 32940, 32941, 32942, 32943, 32944, 32945, 32946, 32947, 32948, 32949 were selected from the ORFs having the nucleotide sequence represented by SEQ ID NO: 2442.

The probes having the nucleotide sequence represented by SEQ ID NOS: 32950, 32951, 32952, 32953, 32954, 32955, 32956, 32957, 32958, 32959, 32960 were selected from the ORFs having the nucleotide sequence represented by SEQ ID NO: 2443.

The probes having the nucleotide sequence represented by SEQ ID NOS: 32961, 32962, 32963, 32964, 32965, 32966, 32967, 32968, 32969, 32970, 32971 were selected from the ORFs having the nucleotide sequence represented by SEQ ID NO: 2444.

The probes having the nucleotide sequence represented by SEQ ID NOS: 32972, 32973, 32974, 32975, 32976, 32977, 32978, 32979, 32980, 32981, 32982 were selected from the ORFs having the nucleotide sequence represented by SEQ ID NO: 2445.

The probes having the nucleotide sequence represented by SEQ ID NOS: 32983, 32984, 32985, 32986, 32987, 32988, 32989, 32990, 32991, 32992, 32993 were selected from the ORFs having the nucleotide sequence represented by SEQ ID NO: 2446.

The probes having the nucleotide sequence represented by SEQ ID NOS: 32994, 32995, 32996, 32997, 32998, 32999, 33000, 33001, 33002, 33003, 33004 were selected from the ORFs having the nucleotide sequence represented by SEQ ID NO: 2447.

The probes having the nucleotide sequence represented by SEQ ID NOS: 33005, 33006, 33007, 33008, 33009, 33010, 33011, 33012, 33013, 33014, 33015 were selected from the ORFs having the nucleotide sequence represented by SEQ ID NO: 2448.

The probes having the nucleotide sequence represented by SEQ ID NOS: 33016, 33017, 33018, 33019, 33020, 33021, 33022, 33023, 33024, 33025, 33026 were selected from the ORFs having the nucleotide sequence represented by SEQ ID NO: 2449.

The probes having the nucleotide sequence represented by SEQ ID NOS: 33027, 33028, 33029, 33030, 33031, 33032, 33033, 33034, 33035, 33036, 33037 were selected from the ORFs having the nucleotide sequence represented by SEQ ID NO: 2450.

The probes having the nucleotide sequence represented by SEQ ID NOS: 33038, 33039, 33040, 33041, 33042, 33043, 33044, 33045, 33046, 33047, 33048 were selected from the ORFs having the nucleotide sequence represented by SEQ ID NO: 2451.

The probes having the nucleotide sequence represented by SEQ ID NOS: 33049, 33050, 33051, 33052, 33053, 33054, 33055, 33056, 33057, 33058, 33059 were selected from the ORFs having the nucleotide sequence represented by SEQ ID NO: 2452.

The probes having the nucleotide sequence represented by SEQ ID NOS: 33060, 33061, 33062, 33063, 33064, 33065, 33066, 33067, 33068, 33069, 33070 were selected from the ORFs having the nucleotide sequence represented by SEQ ID NO: 2453.

The probes having the nucleotide sequence represented by SEQ ID NOS: 33071, 33072, 33073, 33074, 33075, 33076, 33077, 33078, 33079, 33080, 33081 were selected from the ORFs having the nucleotide sequence represented by SEQ ID NO: 2454.

The probes having the nucleotide sequence represented by SEQ ID NOS: 33082, 33083, 33084, 33085, 33086, 33087, 33088, 33089, 33090, 33091, 33092 were selected from the ORFs having the nucleotide sequence represented by SEQ ID NO: 2455.

The probes having the nucleotide sequence represented by SEQ ID NOS: 33093, 33094, 33095, 33096, 33097, 33098, 33099, 33100, 33101, 33102, 33103 were selected from the ORFs having the nucleotide sequence represented by SEQ ID NO: 2456.

The probes having the nucleotide sequence represented by SEQ ID NOS: 33104, 33105, 33106, 33107, 33108, 33109, 33110, 33111, 33112, 33113, 33114 were selected from the ORFs having the nucleotide sequence represented by SEQ ID NO: 2457.

The probes having the nucleotide sequence represented by SEQ ID NOS: 33115, 33116, 33117, 33118, 33119, 33120, 33121, 33122, 33123, 33124, 33125 were selected from the ORFs having the nucleotide sequence represented by SEQ ID NO: 2458.

The probes having the nucleotide sequence represented by SEQ ID NOS: 33126, 33127, 33128, 33129, 33130, 33131, 33132, 33133, 3313.4, 33135, 33136 were selected from the ORFs having the nucleotide sequence represented by SEQ ID NO: 2459.

The probes having the nucleotide sequence represented by SEQ ID NOS: 33137, 33138, 33139, 33140, 33141, 33142, 33143, 33144, 33145, 33146, 33147 were selected from the ORFs having the nucleotide sequence represented by SEQ ID NO: 2460.

The probes having the nucleotide sequence represented by SEQ ID NOS: 33148, 33149, 33150, 33151, 33152, 33153, 33154, 33155, 33156, 33157, 33158 were selected from the ORFs having the nucleotide sequence represented by SEQ ID NO: 2461.

The probes having the nucleotide sequence represented by SEQ ID NOS: 33159, 33160, 33161, 33162, 33163, 33164, 33165, 33166, 33167, 33168, 33169 were selected from the ORFs having the nucleotide sequence represented by SEQ ID NO: 2462.

The probes having the nucleotide sequence represented by SEQ ID NOS: 33170, 33171, 33172, 33173, 33174, 33175, 33176, 33177, 33178, 33179, 33180 were selected from the ORFs having the nucleotide sequence represented by SEQ ID NO: 2463.

The probes having the nucleotide sequence represented by SEQ ID NOS: 33181, 33182, 33183, 33184, 33185, 33186, 33187, 33188, 33189, 33190, 33191 were selected from the ORFs having the nucleotide sequence represented by SEQ ID NO: 2464.

The probes having the nucleotide sequence represented by SEQ ID NOS: 33192, 33193, 33194, 33195, 33196, 33197, 33198, 33199, 33200, 33201, 33202 were selected from the ORFs having the nucleotide sequence represented by SEQ ID NO: 2465.

The probes having the nucleotide sequence represented by SEQ ID NOS: 33203, 33204, 33205, 33206, 33207, 33208, 33209, 33210, 33211, 33212, 33213 were selected from the ORFs having the nucleotide sequence represented by SEQ ID NO: 2466.

The probes having the nucleotide sequence represented by SEQ ID NOS: 33214, 33215, 33216, 33217, 33218, 33219, 33220, 33221, 33222, 33223, 33224 were selected from the ORFs having the nucleotide sequence represented by SEQ ID NO: 2467.

The probes having the nucleotide sequence represented by SEQ ID NOS: 33225, 33226, 33227, 33228, 33229, 33230, 33231, 33232, 33233, 33234, 33235 were selected from the ORFs having the nucleotide sequence represented by SEQ ID NO: 2468.

The probes having the nucleotide sequence represented by SEQ ID NOS: 33236, 33237, 33238, 33239, 33240, 33241, 33242, 33243, 33244, 33245, 33246 were selected from the ORFs having the nucleotide sequence represented by SEQ ID NO: 2469.

The probes having the nucleotide sequence represented by SEQ ID NOS: 33247, 33248, 33249, 33250, 33251, 33252, 33253, 33254, 33255, 33256, 33257 were selected from the ORFs having the nucleotide sequence represented by SEQ ID NO: 2470.

The probes having the nucleotide sequence represented by SEQ ID NOS: 33258, 33259, 33260, 33261, 33262, 33263, 33264, 33265, 33266, 33267, 33268 were selected from the ORFs having the nucleotide sequence represented by SEQ ID NO: 2471.

The probes having the nucleotide sequence represented by SEQ ID NOS: 33269, 33270, 33271, 33272, 33273, 33274, 33275, 33276, 33277, 33278, 33279 were selected from the ORFs having the nucleotide sequence represented by SEQ ID NO: 2472.

The probes having the nucleotide sequence represented by SEQ ID NOS: 33280, 33281, 33282, 33283, 33284, 33285, 33286, 33287, 33288, 33289, 33290 were selected from the ORFs having the nucleotide sequence represented by SEQ ID NO: 2473.

The probes having the nucleotide sequence represented by SEQ ID NOS: 33291, 33292, 33293, 33294, 33295, 33296, 33297, 33298, 33299, 33300, 33301 were selected from the ORFs having the nucleotide sequence represented by SEQ ID NO: 2474.

The probes having the nucleotide sequence represented by SEQ ID NOS: 33302, 33303, 33304, 33305, 33306, 33307, 33308, 33309, 33310, 33311, 33312 were selected from the ORFs having the nucleotide sequence represented by SEQ ID NO: 2475.

The probes having the nucleotide sequence represented by SEQ ID NOS: 33313, 33314, 33315, 33316, 33317, 33318, 33319, 33320, 33321, 33322, 33323 were selected from the ORFs having the nucleotide sequence represented by SEQ ID NO: 2476.

The probes having the nucleotide sequence represented by SEQ ID NOS: 33324, 33325, 33326, 33327, 33328, 33329, 33330, 33331, 33332, 33333, 33334 were selected from the ORFs having the nucleotide sequence represented by SEQ ID NO: 2477.

The probes having the nucleotide sequence represented by SEQ ID NOS: 33335, 33336, 33337, 33338, 33339, 33340, 33341, 33342, 33343, 33344, 33345 were selected from the ORFs having the nucleotide sequence represented by SEQ ID NO: 2478.

The probes having the nucleotide sequence represented by SEQ ID NOS: 33346, 33347, 33348, 33349, 33350, 33351, 33352, 33353, 33354, 33355, 33356 were selected from the ORFs having the nucleotide sequence represented by SEQ ID NO: 2479.

The probes having the nucleotide sequence represented by SEQ ID NOS: 33357, 33358, 33359, 33360, 33361, 33362, 33363, 33364, 33365, 333.66, 33367 were selected from the ORFs having the nucleotide sequence represented by SEQ ID NO: 2480.

The probes having the nucleotide sequence represented by SEQ ID NOS: 33368, 33369, 33370, 33371, 33372, 33373, 33374, 33375, 33376, 33377, 33378 were selected from the ORFs having the nucleotide sequence represented by SEQ ID NO: 2481.

The probes having the nucleotide sequence represented by SEQ ID NOS: 33379, 33380, 33381, 33382, 33383, 33384, 33385, 33386, 33387, 33388, 33389 were selected from the ORFs having the nucleotide sequence represented by SEQ ID NO: 2482.

The probes having the nucleotide sequence represented by SEQ ID NOS: 33390, 33391, 33392, 33393, 33394, 33395, 33396, 33397, 33398, 33399, 33400 were selected from the ORFs having the nucleotide sequence represented by SEQ ID NO: 2483.

The probes having the nucleotide sequence represented by SEQ ID NOS: 33401, 33402, 33403, 33404, 33405, 33406, 33407, 33408, 33409, 33410, 33411 were selected from the ORFs having the nucleotide sequence represented by SEQ ID NO: 2484.

The probes having the nucleotide sequence represented by SEQ ID NOS: 33412, 33413, 33414, 33415, 33416, 33417, 33418, 33419, 33420, 33421, 33422 were selected from the ORFs having the nucleotide sequence represented by SEQ ID NO: 2485.

The probes having the nucleotide sequence represented by SEQ ID NOS: 33423, 33424, 33425, 33426, 33427, 33428, 33429, 33430, 33431, 33432, 33433 were selected from the ORFs having the nucleotide sequence represented by SEQ ID NO: 2486.

The probes having the nucleotide sequence represented by SEQ ID NOS: 33434, 33435, 33436, 33437, 33438, 33439, 33440, 33441, 33442, 33443, 33444 were selected from the ORFs having the nucleotide sequence represented by SEQ ID NO: 2487.

The probes having the nucleotide sequence represented by SEQ ID NOS: 33445, 33446, 33447, 33448, 33449, 33450, 33451, 33452, 33453, 33454, 33455 were selected from the ORFs having the nucleotide sequence represented by SEQ ID NO: 2488.

The probes having the nucleotide sequence represented by SEQ ID NOS: 33456, 33457, 33458, 33459, 33460, 33461, 33462, 33463, 33464, 33465, 33466 were selected from the ORFs having the nucleotide sequence represented by SEQ ID NO: 2489.

The probes having the nucleotide sequence represented by SEQ ID NOS: 33467, 33468, 33469, 33470, 33471, 33472, 33473, 33474 were selected from the ORFs having the nucleotide sequence represented by SEQ ID NO: 2490.

The probes having the nucleotide sequence represented by SEQ ID NOS: 33475, 33476, 33477, 33478, 33479, 33480, 33481, 33482, 33483, 33484, 33485 were selected from the ORFs having the nucleotide sequence represented by SEQ ID NO: 2491.

The probes having the nucleotide sequence represented by SEQ ID NOS: 33486, 33487, 33488, 33489, 33490, 33491, 33492, 33493, 33494, 33495, 33496 were selected from the ORFs having the nucleotide sequence represented by SEQ ID NO: 2492.

The probes having the nucleotide sequence represented by SEQ ID NOS: 33497, 33498, 33499, 33500, 33501, 33502, 33503, 33504, 33505, 33506, 33507 were selected from the ORFs having the nucleotide sequence represented by SEQ ID NO: 2493.

The probes having the nucleotide sequence represented by SEQ ID NOS: 33508, 33509, 33510, 33511, 33512, 33513, 33514, 33515, 33516, 33517, 33518 were selected from the ORFs having the nucleotide sequence represented by SEQ ID NO: 2494.

The probes having the nucleotide sequence represented by SEQ ID NOS: 33519, 33520, 33521, 33522, 33523, 33524, 33525, 33526, 33527, 33528, 33529 were selected from the ORFs having the nucleotide sequence represented by SEQ ID NO: 2495.

The probes having the nucleotide sequence represented by SEQ ID NOS: 33530, 33531, 33532, 33533, 33534, 33535, 33536, 33537, 33538, 33539, 33540 were selected from the ORFs having the nucleotide sequence represented by SEQ ID NO: 2496.

The probes having the nucleotide sequence represented by SEQ ID NOS: 33541, 33542, 33543, 33544, 33545, 33546, 33547, 33548, 33549, 33550, 33551 were selected from the ORFs having the nucleotide sequence represented by SEQ ID NO: 2497.

The probes having the nucleotide sequence represented by SEQ ID NOS: 33552, 33553, 33554, 33555, 33556, 33557, 33558, 33559, 33560, 33561, 33562 were selected from the ORFs having the nucleotide sequence represented by SEQ ID NO: 2498.

The probes having the nucleotide sequence represented by SEQ ID NOS: 33563, 33564, 33565, 33566, 33567, 33568, 33569, 33570, 33571, 33572, 33573 were selected from the ORFs having the nucleotide sequence represented by SEQ ID NO: 2499.

The probes having the nucleotide sequence represented by SEQ ID NOS: 33574, 33575, 33576, 33577, 33578, 33579, 33580, 33581, 33582, 33583, 33584 were selected from the ORFs having the nucleotide sequence represented by SEQ ID NO: 2500.

The probes having the nucleotide sequence represented by SEQ ID NOS: 33585, 33586, 33587, 33588, 33589, 33590, 33591, 33592, 33593, 33594, 33595 were selected from the ORFs having the nucleotide sequence represented by SEQ ID NO: 2501.

The probes having the nucleotide sequence represented by SEQ ID NOS: 33596, 33597, 33598, 33599, 33600, 33601, 33602, 33603, 33604, 33605, 33606 were selected from the ORFs having the nucleotide sequence represented by SEQ ID NO: 2502.

The probes having the nucleotide sequence represented by SEQ ID NOS: 33607, 33608, 33609, 33610, 33611, 33612, 33613, 33614, 33615, 33616, 33617 were selected from the ORFs having the nucleotide sequence represented by SEQ ID NO: 2503.

The probes having the nucleotide sequence represented by SEQ ID NOS: 33618, 33619, 33620, 33621, 33622, 33623, 33624, 33625, 33626, 33627, 33628 were selected from the ORFs having the nucleotide sequence represented by SEQ ID NO: 2504.

The probes having the nucleotide sequence represented by SEQ ID NOS: 33629, 33630, 33631, 33632, 33633, 33634, 33635, 33636, 33637, 33638, 33639 were selected from the ORFs having the nucleotide sequence represented by SEQ ID NO: 2505.

The probes having the nucleotide sequence represented by SEQ ID NOS: 33640, 33641, 33642, 33643, 33644, 33645, 33646, 33647, 33648, 33649, 33650 were selected from the ORFs having the nucleotide sequence represented by SEQ ID NO: 2506.

The probes having the nucleotide sequence represented by SEQ ID NOS: 33651, 33652, 33653, 33654, 33655, 33656, 33657, 33658, 33659, 33660, 33661 were selected from the ORFs having the nucleotide sequence represented by SEQ ID NO: 2507.

The probes having the nucleotide sequence represented by SEQ ID NOS: 33662, 33663, 33664, 33665, 33666, 33667, 33668, 33669, 33670, 33671, 33672 were selected from the ORFs having the nucleotide sequence represented by SEQ ID NO: 2508.

The probes having the nucleotide sequence represented by SEQ ID NOS: 33673, 33674, 33675, 33676, 33677, 33678, 33679, 33680, 33681, 33682, 33683 were selected from the ORFs having the nucleotide sequence represented by SEQ ID NO: 2509.

The probes having the nucleotide sequence represented by SEQ ID NOS: 33684, 33685, 33686, 33687, 33688, 33689, 33690, 33691, 33692, 33693, 33694 were selected from the ORFs having the nucleotide sequence represented by SEQ ID NO: 2510.

The probes having the nucleotide sequence represented by SEQ ID NOS: 33695, 33696, 33697, 33698, 33699, 33700, 33701, 33702, 33703, 33704, 33705 were selected from the ORFs having the nucleotide sequence represented by SEQ ID NO: 2511.

The probes having the nucleotide sequence represented by SEQ ID NOS: 33706, 33707, 33708, 33709, 33710, 33711, 33712, 33713, 33714, 33715, 33716 were selected from the ORFs having the nucleotide sequence represented by SEQ ID NO: 2512.

The probes having the nucleotide sequence represented by SEQ ID NOS: 33717, 33718, 33719, 33720, 33721, 33722, 33723, 33724, 33725, 33726, 33727 were selected from the ORFs having the nucleotide sequence represented by SEQ ID NO: 2513.

The probes having the nucleotide sequence represented by SEQ ID NOS: 33728, 33729, 33730, 33731, 33732, 33733, 33734, 33735, 33736, 33737, 33738 were selected from the ORFs having the nucleotide sequence represented by SEQ ID NO: 2514.

The probes having the nucleotide sequence represented by SEQ ID NOS: 33739, 33740, 33741, 33742, 33743, 33744, 33745, 33746, 33747, 33748, 33749 were selected from the ORFs having the nucleotide sequence represented by SEQ ID NO: 2515.

The probes having the nucleotide sequence represented by SEQ ID NOS: 33750, 33751, 33752, 33753, 33754, 33755, 33756, 33757, 33758, 33759, 33760 were selected from the ORFs having the nucleotide sequence represented by SEQ ID NO: 2516.

The probes having the nucleotide sequence represented by SEQ ID NOS: 33761, 33762, 33763, 33764, 33765, 33766, 33767, 33768, 33769, 33770, 33771 were selected from the ORFs having the nucleotide sequence represented by SEQ ID NO: 2517.

The probes having the nucleotide sequence represented by SEQ ID NOS: 33772, 33773, 33774, 33775, 33776, 33777, 33778, 33779, 33780, 33781, 33782 were selected from the ORFs having the nucleotide sequence represented by SEQ ID NO: 2518.

The probes having the nucleotide sequence represented by SEQ ID NOS: 33783, 33784, 33785, 33786, 33787, 33788, 33789, 33790, 33791, 33792, 33793 were selected from the ORFs having the nucleotide sequence represented by SEQ ID NO: 2519.

The probes having the nucleotide sequence represented by SEQ ID NOS: 33794, 33795, 33796, 33797, 33798, 33799, 33800, 33801, 33802, 33803, 33804 were selected from the ORFs having the nucleotide sequence represented by SEQ ID NO: 2520.

The probes having the nucleotide sequence represented by SEQ ID NOS: 33805, 33806, 33807, 33808, 33809, 33810, 33811, 33812, 33813, 33814, 33815 were selected from the ORFs having the nucleotide sequence represented by SEQ ID NO: 2521.

The probes having the nucleotide sequence represented by SEQ ID NOS: 33816, 33817, 33818, 33819, 33820, 33821, 33822, 33823, 33824, 33825, 33826 were selected from the ORFs having the nucleotide sequence represented by SEQ ID NO: 2522.

The probes having the nucleotide sequence represented by SEQ ID NOS: 33827, 33828, 33829, 33830, 33831, 33832, 33833, 33834, 33835, 33836, 33837 were selected from the ORFs having the nucleotide sequence represented by SEQ ID NO: 2523.

The probes having the nucleotide sequence represented by SEQ ID NOS: 33838, 33839, 33840, 33841, 33842, 33843, 33844, 33845, 33846, 33847, 33848 were selected from the ORFs having the nucleotide sequence represented by SEQ ID NO: 2524.

The probes having the nucleotide sequence represented by SEQ ID NOS: 33849, 33850, 33851, 33852, 33853, 33854, 33855, 33856, 33857, 33858, 33859 were selected from the ORFs having the nucleotide sequence represented by SEQ ID NO: 2525.

The probes having the nucleotide sequence represented by SEQ ID NOS: 33860, 33861, 33862, 33863, 33864, 33865, 33866, 33867, 33868, 33869, 33870 were selected from the ORFs having the nucleotide sequence represented by SEQ ID NO: 2526.

The probes having the nucleotide sequence represented by SEQ ID NOS: 33871, 33872, 33873, 33874, 33875, 33876, 33877, 33878, 33879, 33880, 33881 were selected from the ORFs having the nucleotide sequence represented by SEQ ID NO: 2527.

The probes having the nucleotide sequence represented by SEQ ID NOS: 33882, 33883, 33884, 33885, 33886, 33887, 33888, 33889, 33890, 33891, 33892 were selected from the ORFs having the nucleotide sequence represented by SEQ ID NO: 2528.

The probes having the nucleotide sequence represented by SEQ ID NOS: 33893, 33894, 33895, 33896, 33897, 33898, 33899, 33900, 33901, 33902, 33903 were selected from the ORFs having the nucleotide sequence represented by SEQ ID NO: 2529.

The probes having the nucleotide sequence represented by SEQ ID NOS: 33904, 33905, 33906, 33907, 33908, 33909, 33910, 33911, 33912, 33913, 33914 were selected from the ORFs having the nucleotide sequence represented by SEQ ID NO: 2530.

The probes having the nucleotide sequence represented by SEQ ID NOS: 33915, 33916, 33917, 33918, 33919, 33920, 33921, 33922, 33923, 33924, 33925 were selected from the ORFs having the nucleotide sequence represented by SEQ ID NO: 2531.

The probes having the nucleotide sequence represented by SEQ ID NOS: 33926, 33927, 33928, 33929, 33930, 33931, 33932, 33933, 33934, 33935, 33936 were selected from the ORFs having the nucleotide sequence represented by SEQ ID NO: 2532.

The probes having the nucleotide sequence represented by SEQ ID NOS: 33937, 33938, 33939, 33940, 33941, 33942, 33943, 33944, 33945, 33946, 33947 were selected from the ORFs having the nucleotide sequence represented by SEQ ID NO: 2533.

The probes having the nucleotide sequence represented by SEQ ID NOS: 33948, 33949, 33950, 33951, 33952, 33953, 33954, 33955, 33956, 33957, 33958 were selected from the ORFs having the nucleotide sequence represented by SEQ ID NO: 2534.

The probes having the nucleotide sequence represented by SEQ ID NOS: 33959, 33960, 33961, 33962, 33963, 33964, 33965, 33966, 33967, 33968, 33969 were selected from the ORFs having the nucleotide sequence represented by SEQ ID NO: 2535.

The probes having the nucleotide sequence represented by SEQ ID NOS: 33970, 33971, 33972, 33973, 33974, 33975, 33976, 33977, 33978, 33979, 33980 were selected from the ORFs having the nucleotide sequence represented by SEQ ID NO: 2536.

The probes having the nucleotide sequence represented by SEQ ID NOS: 33981, 33982, 33983, 33984, 33985, 33986, 33987, 33988, 33989, 33990, 33991 were selected from the ORFs having the nucleotide sequence represented by SEQ ID NO: 2537.

The probes having the nucleotide sequence represented by SEQ ID NOS: 33992, 33993, 33994, 33995, 33996, 33997, 33998, 33999, 34000, 34001, 34002 were selected from the ORFs having the nucleotide sequence represented by SEQ ID NO: 2538.

The probes having the nucleotide sequence represented by SEQ ID NOS: 34003, 34004, 34005, 34006, 34007, 34008, 34009, 34010, 34011, 34012, 34013 were selected from the ORFs having the nucleotide sequence represented by SEQ ID NO: 2539.

The probes having the nucleotide sequence represented by SEQ ID NOS: 34014, 34015, 34016, 34017, 34018, 34019, 34020, 34021, 34022, 34023, 34024 were selected from the ORFs having the nucleotide sequence represented by SEQ ID NO: 2540.

The probes having the nucleotide sequence represented by SEQ ID NOS: 34025, 34026, 34027, 34028, 34029, 34030, 34031, 34032, 34033, 34034, 34035 were selected from the ORFs having the nucleotide sequence represented by SEQ ID NO: 2541.

The probes having the nucleotide sequence represented by SEQ ID NOS: 34036, 34037, 34038, 34039, 34040, 34041, 34042, 34043, 34044, 34045, 34046 were selected from the ORFs having the nucleotide sequence represented by SEQ ID NO: 2542.

The probes having the nucleotide sequence represented by SEQ ID NOS: 34047, 34048, 34049, 34050, 34051, 34052, 34053, 34054, 34055, 34056, 34057 were selected from the ORFs having the nucleotide sequence represented by SEQ ID NO: 2543.

The probes having the nucleotide sequence represented by SEQ ID NOS: 34058, 34059, 34060, 34061, 34062, 34063, 34064, 34065, 34066, 34067, 34068 were selected from the ORFs having the nucleotide sequence represented by SEQ ID NO: 2544.

The probes having the nucleotide sequence represented by SEQ ID NOS: 34069, 34070, 34071, 34072, 34073, 34074, 34075, 34076, 34077, 34078, 34079 were selected from the ORFs having the nucleotide sequence represented by SEQ ID NO: 2545.

The probes having the nucleotide sequence represented by SEQ ID NOS: 34080, 34081, 34082, 34083, 34084, 34085, 34086, 34087, 34088, 34089, 34090 were selected from the ORFs having the nucleotide sequence represented by SEQ ID NO: 2546.

The probes having the nucleotide sequence represented by SEQ ID NOS: 34091, 34092, 34093, 34094, 34095, 34096, 34097, 34098, 34099, 34100, 34101 were selected from the ORFs having the nucleotide sequence represented by SEQ ID NO: 2547.

The probes having the nucleotide sequence represented by SEQ ID NOS: 34102, 34103, 34104, 34105, 34106, 34107, 34108, 34109, 34110, 34111, 34112 were selected from the ORFs having the nucleotide sequence represented by SEQ ID NO: 2548.

The probes having the nucleotide sequence represented by SEQ ID NOS: 34113, 34114, 34115, 34116, 34117, 34118, 34119, 34120, 34121, 34122, 34123 were selected from the ORFs having the nucleotide sequence represented by SEQ ID NO: 2549.

The probes having the nucleotide sequence represented by SEQ ID NOS: 34124, 34125, 34126, 34127, 34128, 34129, 34130, 34131, 34132, 34133, 34134 were selected from the ORFs having the nucleotide sequence represented by SEQ ID NO: 2550.

The probes having the nucleotide sequence represented by SEQ ID NOS: 34135, 34136, 34137, 34138, 34139, 34140, 34141, 34142, 34143, 34144, 34145 were selected from the ORFs having the nucleotide sequence represented by SEQ ID NO: 2551.

The probes having the nucleotide sequence represented by SEQ ID NOS: 34146, 34147, 34148, 34149, 34150, 34151, 34152, 34153, 34154, 34155, 34156 were selected from the ORFs having the nucleotide sequence represented by SEQ ID NO: 2552.

The probes having the nucleotide sequence represented by SEQ ID NOS: 34157, 34158, 34159, 34160, 34161, 34162, 34163, 34164, 34165, 34166, 34167 were selected from the ORFs having the nucleotide sequence represented by SEQ ID NO: 2553.

The probes having the nucleotide sequence represented by SEQ ID NOS: 34168, 34169, 34170, 34171, 34172, 34173, 34174, 34175, 34176, 34177, 34178 were selected from the ORFs having the nucleotide sequence represented by SEQ ID NO: 2554.

The probes having the nucleotide sequence represented by SEQ ID NOS: 34179, 34180, 34181, 34182, 34183, 34184, 34185, 34186, 34187, 34188, 34189 were selected from the ORFs having the nucleotide sequence represented by SEQ ID NO: 2555.

The probes having the nucleotide sequence represented by SEQ ID NOS: 34190, 34191, 34192, 34193, 34194, 34195, 34196, 34197, 34198, 34199, 34200 were selected from the ORFs having the nucleotide sequence represented by SEQ ID NO: 2556.

The probes having the nucleotide sequence represented by SEQ ID NOS: 34201, 34202, 34203, 34204, 34205, 34206, 34207, 34208, 34209, 34210, 34211 were selected from the ORFs having the nucleotide sequence represented by SEQ ID NO: 2557.

The probes having the nucleotide sequence represented by SEQ ID NOS: 34212, 34213, 34214, 34215, 34216, 34217, 34218, 34219, 34220, 34221, 34222, 34223, 34224, 34225, 34226, 34227, 34228, 34229, 34230, 34231, 34232, 34233 were selected from the ORFs having the nucleotide sequence represented by SEQ ID NO: 2558.

The probes having the nucleotide sequence represented by SEQ ID NOS: 34234, 34235, 34236, 34237, 34238, 34239, 34240, 34241, 34242, 34243, 34244 were selected from the ORFs having the nucleotide sequence represented by SEQ ID NO: 2559.

The probes having the nucleotide sequence represented by SEQ ID NOS: 34245, 34246, 34247, 34248, 34249, 34250, 34251, 34252, 34253, 34254, 34255 were selected from the ORFs having the nucleotide sequence represented by SEQ ID NO: 2560.

The probes having the nucleotide sequence represented by SEQ ID NOS: 34256, 34257, 34258, 34259, 34260, 34261, 34262, 34263, 34264, 34265, 34266 were selected from the ORFs having the nucleotide sequence represented by SEQ ID NO: 2561.

The probes having the nucleotide sequence represented by SEQ ID NOS: 34267, 34268, 34269, 34270, 34271, 34272, 34273, 34274, 34275, 34276, 34277 were selected from the ORFs having the nucleotide sequence represented by SEQ ID NO: 2562.

The probes having the nucleotide sequence represented by SEQ ID NOS: 34278, 34279, 34280, 34281, 34282, 34283, 34284, 34285, 34286, 34287, 34288 were selected from the ORFs having the nucleotide sequence represented by SEQ ID NO: 2563.

The probes having the nucleotide sequence represented by SEQ ID NOS: 34289, 34290, 34291, 34292, 34293, 34294, 34295, 34296, 34297, 34298, 34299 were selected from the ORFs having the nucleotide sequence represented by SEQ ID NO: 2564.

The probes having the nucleotide sequence represented by SEQ ID NOS: 34300, 34301, 34302, 34303, 34304, 34305, 34306, 34307, 34308, 34309, 34310 were selected from the ORFs having the nucleotide sequence represented by SEQ ID NO: 2565.

The probes having the nucleotide sequence represented by SEQ ID NOS: 34311, 34312, 34313, 34314, 34315, 34316, 34317, 34318, 34319, 34320, 34321 were selected from the ORFs having the nucleotide sequence represented by SEQ ID NO: 2566.

The probes having the nucleotide sequence represented by SEQ ID NOS: 34322, 34323, 34324, 34325, 34326, 34327, 34328, 34329, 34330, 34331, 34332 were selected from the ORFs having the nucleotide sequence represented by SEQ ID NO: 2567.

The probes having the nucleotide sequence represented by SEQ ID NOS: 34333, 34334, 34335, 34336, 34337, 34338, 34339, 34340, 34341, 34342, 34343 were selected from the ORFs having the nucleotide sequence represented by SEQ ID NO: 2568.

The probes having the nucleotide sequence represented by SEQ ID NOS: 34344, 34345, 34346, 34347, 34348, 34349, 34350, 34351, 34352, 34353, 34354 were selected from the ORFs having the nucleotide sequence represented by SEQ ID NO: 2569.

The probes having the nucleotide sequence represented by SEQ ID NOS: 34355, 34356, 34357, 34358, 34359, 34360, 34361, 34362, 34363, 34364, 34365 were selected from the ORFs having the nucleotide sequence represented by SEQ ID NO: 2570.

The probes having the nucleotide sequence represented by SEQ ID NOS: 34366, 34367, 34368, 34369, 34370, 34371, 34372, 34373, 34374, 34375, 34376 were selected from the ORFs having the nucleotide sequence represented by SEQ ID NO: 2571.

The probes having the nucleotide sequence represented by SEQ ID NOS: 34377, 34378, 34379, 34380, 34381, 34382, 34383, 34384, 34385, 34386, 34387 were selected from the ORFs having the nucleotide sequence represented by SEQ ID NO: 2572.

The probes having the nucleotide sequence represented by SEQ ID NOS: 34388, 34389, 34390, 34391, 34392, 34393, 34394, 34395, 34396, 34397, 34398 were selected from the ORFs having the nucleotide sequence represented by SEQ ID NO: 2573.

The probes having the nucleotide sequence represented by SEQ ID NOS: 34399, 34400, 34401, 34402, 34403, 34404, 34405, 34406, 34407, 34408, 34409 were selected from the ORFs having the nucleotide sequence represented by SEQ ID NO: 2574.

The probes having the nucleotide sequence represented by SEQ ID NOS: 34410, 34411, 34412, 34413, 34414, 34415, 34416, 34417, 34418, 34419, 34420 were selected from the ORFs having the nucleotide sequence represented by SEQ ID NO: 2575.

The probes having the nucleotide sequence represented by SEQ ID NOS: 34421, 34422, 34423, 34424, 34425, 34426, 34427, 34428, 34429, 34430, 34431 were selected from the ORFs having the nucleotide sequence represented by SEQ ID NO: 2576.

The probes having the nucleotide sequence represented by SEQ ID NOS: 34432, 34433, 34434, 34435, 34436, 34437, 34438, 34439, 34440, 34441, 34442 were selected from the ORFs having the nucleotide sequence represented by SEQ ID NO: 2577.

The probes having the nucleotide sequence represented by SEQ ID NOS: 34443, 34444, 34445, 34446, 34447, 34448, 34449, 34450, 34451, 34452, 34453 were selected from the ORFs having the nucleotide sequence represented by SEQ ID NO: 2578.

The probes having the nucleotide sequence represented by SEQ ID NOS: 34454, 34455, 34456, 34457, 34458, 34459, 34460, 34461, 34462, 34463, 34464 were selected from the ORFs having the nucleotide sequence represented by SEQ ID NO: 2579.

The probes having the nucleotide sequence represented by SEQ ID NOS: 34465, 34466, 34467, 34468, 34469, 34470, 34471, 34472, 34473, 34474, 34475 were selected from the ORFs having the nucleotide sequence represented by SEQ ID NO: 2580.

The probes having the nucleotide sequence represented by SEQ ID NOS: 34476, 34477, 34478, 34479, 34480, 34481, 34482, 34483, 34484, 34485, 34486 were selected from the ORFs having the nucleotide sequence represented by SEQ ID NO: 2581.

The probes having the nucleotide sequence represented by SEQ ID NOS: 34487, 34488, 34489, 34490, 34491, 34492, 34493, 34494, 34495, 34496, 34497 were selected from the ORFs having the nucleotide sequence represented by SEQ ID NO: 2582.

The probes having the nucleotide sequence represented by SEQ ID NOS: 34498, 34499, 34500, 34501, 34502, 34503, 34504, 34505, 34506, 34507, 34508 were selected from the ORFs having the nucleotide sequence represented by SEQ ID NO: 2583.

The probes having the nucleotide sequence represented by SEQ ID NOS: 34509, 34510, 34511, 34512, 34513, 34514, 34515, 34516, 34517, 34518, 34519 were selected from the ORFs having the nucleotide sequence represented by SEQ ID NO: 2584.

The probes having the nucleotide sequence represented by SEQ ID NOS: 34520, 34521, 34522, 34523, 34524, 34525, 34526, 34527, 34528, 34529, 34530 were selected from the ORFs having the nucleotide sequence represented by SEQ ID NO: 2585.

The probes having the nucleotide sequence represented by SEQ ID NOS: 34531, 34532, 34533, 34534, 34535, 34536, 34537, 34538, 34539, 34540, 34541 were selected from the ORFs having the nucleotide sequence represented by SEQ ID NO: 2586.

The probes having the nucleotide sequence represented by SEQ ID NOS: 34542, 34543, 34544, 34545, 34546, 34547, 34548, 34549, 34550, 34551, 34552 were selected from the ORFs having the nucleotide sequence represented by SEQ ID NO: 2587.

The probes having the nucleotide sequence represented by SEQ ID NOS: 34553, 34554, 34555, 34556, 34557, 34558, 34559, 34560, 34561, 34562, 34563 were selected from the ORFs having the nucleotide sequence represented by SEQ ID NO: 2588.

The probes having the nucleotide sequence represented by SEQ ID NOS: 34564, 34565, 34566, 34567, 34568, 34569, 34570, 34571, 34572, 34573, 34574 were selected from the ORFs having the nucleotide sequence represented by SEQ ID NO: 2589.

The probes having the nucleotide sequence represented by SEQ ID NOS: 34575, 34576, 34577, 34578, 34579, 34580, 34581, 34582, 34583, 34584, 34585 were selected from the ORFs having the nucleotide sequence represented by SEQ ID NO: 2590.

The probes having the nucleotide sequence represented by SEQ ID NOS: 34586, 34587, 34588, 34589, 34590, 34591, 34592, 34593, 34594, 34595, 34596 were selected from the ORFs having the nucleotide sequence represented by SEQ ID NO: 2591.

The probes having the nucleotide sequence represented by SEQ ID NOS: 34597, 34598, 34599, 34600, 34601, 34602, 34603, 34604, 34605, 34606, 34607 were selected from the ORFs having the nucleotide sequence represented by SEQ ID NO: 2592.

The probes having the nucleotide sequence represented by SEQ ID NOS: 34608, 34609, 34610, 34611, 34612, 34613, 34614, 34615, 34616, 34617, 34618 were selected from the ORFs having the nucleotide sequence represented by SEQ ID NO: 2593.

The probes having the nucleotide sequence represented by SEQ ID NOS: 34619, 34620, 34621, 34622, 34623, 34624, 34625, 34626, 34627, 34628, 34629 were selected from the ORFs having the nucleotide sequence represented by SEQ ID NO: 2594.

The probes having the nucleotide sequence represented by SEQ ID NOS: 34630, 34631, 34632, 34633, 34634, 34635, 34636, 34637, 34638, 34639, 34640 were selected from the ORFs having the nucleotide sequence represented by SEQ ID NO: 2595.

The probes having the nucleotide sequence represented by SEQ ID NOS: 34641, 34642, 34643, 34644, 34645, 34646, 34647, 34648, 34649, 34650, 34651 were selected from the ORFs having the nucleotide sequence represented by SEQ ID NO: 2596.

The probes having the nucleotide sequence represented by SEQ ID NOS: 34652, 34653, 34654, 34655, 34656, 34657, 34658, 34659, 34660, 34661, 34662 were selected from the ORFs having the nucleotide sequence represented by SEQ ID NO: 2597.

The probes having the nucleotide sequence represented by SEQ ID NOS: 34663, 34664, 34665, 34666, 34667, 34668, 34669, 34670, 34671, 34672, 34673 were selected from the ORFs having the nucleotide sequence represented by SEQ ID NO: 2598.

The probes having the nucleotide sequence represented by SEQ ID NOS: 34674, 34675, 34676, 34677, 34678, 34679, 34680, 34681, 34682, 34683, 34684 were selected from the ORFs having the nucleotide sequence represented by SEQ ID NO: 2599.

The probes having the nucleotide sequence represented by SEQ ID NOS: 34685, 34686, 34687, 34688, 34689, 34690, 34691, 34692, 34693, 34694, 34695 were selected from the ORFs having the nucleotide sequence represented by SEQ ID NO: 2600.

The probes having the nucleotide sequence represented by SEQ ID NOS: 34696, 34697, 34698, 34699, 34700, 34701, 34702, 34703, 34704, 34705, 34706 were selected from the ORFs having the nucleotide sequence represented by SEQ ID NO: 2601.

The probes having the nucleotide sequence represented by SEQ ID NOS: 34707, 34708, 34709, 34710, 34711, 34712, 34713, 34714, 34715, 34716, 34717 were selected from the ORFs having the nucleotide sequence represented by SEQ ID NO: 2602.

The probes having the nucleotide sequence represented by SEQ ID NOS: 34718, 34719, 34720, 34721, 34722, 34723, 34724, 34725, 34726, 34727, 34728 were selected from the ORFs having the nucleotide sequence represented by SEQ ID NO: 2603.

The probes having the nucleotide sequence represented by SEQ ID NOS: 34729, 34730, 34731, 34732, 34733, 34734, 34735, 34736, 34737, 34738, 34739 were selected from the ORFs having the nucleotide sequence represented by SEQ ID NO: 2604.

The probes having the nucleotide sequence represented by SEQ ID NOS: 34740, 34741, 34742, 34743, 34744, 34745, 34746, 34747, 34748, 34749, 34750 were selected from the ORFs having the nucleotide sequence represented by SEQ ID NO: 2605.

The probes having the nucleotide sequence represented by SEQ ID NOS: 34751, 34752, 34753, 34754, 34755, 34756, 34757, 34758, 34759, 34760, 34761 were selected from the ORFs having the nucleotide sequence represented by SEQ ID NO: 2606.

The probes having the nucleotide sequence represented by SEQ ID NOS: 34762, 34763, 34764, 34765, 34766, 34767, 34768, 34769, 34770, 34771, 34772 were selected from the ORFs having the nucleotide sequence represented by SEQ ID NO: 2607.

The probes having the nucleotide sequence represented by SEQ ID NOS: 34773, 34774, 34775, 34776, 34777, 34778, 34779, 34780, 34781, 34782, 34783 were selected from the ORFs having the nucleotide sequence represented by SEQ ID NO: 2608.

The probes having the nucleotide sequence represented by SEQ ID NOS: 34784, 34785, 34786, 34787, 34788, 34789, 34790, 34791, 34792, 34793, 34794 were selected from the ORFs having the nucleotide sequence represented by SEQ ID NO: 2609.

The probes having the nucleotide sequence represented by SEQ ID NOS: 34795, 34796, 34797, 34798, 34799, 34800, 34801, 34802, 34803, 34804, 34805 were selected from the ORFs having the nucleotide sequence represented by SEQ ID NO: 2610.

The probes having the nucleotide sequence represented by SEQ ID NOS: 34806, 34807, 34808, 34809, 34810, 34811, 34812, 34813, 34814, 34815, 34816 were selected from the ORFs having the nucleotide sequence represented by SEQ ID NO: 2611.

The probes having the nucleotide sequence represented by SEQ ID NOS: 34817, 34818, 34819, 34820, 34821, 34822, 34823, 34824, 34825, 34826, 34827 were selected from the ORFs having the nucleotide sequence represented by SEQ ID NO: 2612.

The probes having the nucleotide sequence represented by SEQ ID NOS: 34828, 34829, 34830, 34831, 34832, 34833, 34834, 34835, 34836, 34837, 34838 were selected from the ORFs having the nucleotide sequence represented by SEQ ID NO: 2613.

The probes having the nucleotide sequence represented by SEQ ID NOS: 34839, 34840, 34841, 34842, 34843, 34844, 34845, 34846, 34847, 34848, 34849 were selected from the ORFs having the nucleotide sequence represented by SEQ ID NO: 2614.

The probes having the nucleotide sequence represented by SEQ ID NOS: 34850, 34851, 34852, 34853, 34854, 34855, 34856, 34857, 34858, 34859, 34860 were selected from the ORFs having the nucleotide sequence represented by SEQ ID NO: 2615.

The probes having the nucleotide sequence represented by SEQ ID NOS: 34861, 34862, 34863, 34864, 34865, 34866, 34867, 34868, 34869, 34870, 34871 were selected from the ORFs having the nucleotide sequence represented by SEQ ID NO: 2616.

The probes having the nucleotide sequence represented by SEQ ID NOS: 34872, 34873, 34874, 34875, 34876, 34877, 34878, 34879, 34880, 34881, 34882 were selected from the ORFs having the nucleotide sequence represented by SEQ ID NO: 2617.

The probes having the nucleotide sequence represented by SEQ ID NOS: 34883, 34884, 34885, 34886, 34887, 34888, 34889, 34890, 34891, 34892, 34893 were selected from the ORFs having the nucleotide sequence represented by SEQ ID NO: 2618.

The probes having the nucleotide sequence represented by SEQ ID NOS: 34894, 34895, 34896, 34897, 34898, 34899, 34900, 34901, 34902, 34903, 34904 were selected from the ORFs having the nucleotide sequence represented by SEQ ID NO: 2619.

The probes having the nucleotide sequence represented by SEQ ID NOS: 34905, 34906, 34907, 34908, 34909, 34910, 34911, 34912, 34913, 34914, 34915 were selected from the ORFs having the nucleotide sequence represented by SEQ ID NO: 2620.

The probes having the nucleotide sequence represented by SEQ ID NOS: 34916, 34917, 34918, 34919, 34920, 34921, 34922, 34923, 34924, 34925, 34926 were selected from the ORFs having the nucleotide sequence represented by SEQ ID NO: 2621.

The probes having the nucleotide sequence represented by SEQ ID NOS: 34927, 34928, 34929, 34930, 34931, 34932, 34933, 34934, 34935, 34936, 34937 were selected from the ORFs having the nucleotide sequence represented by SEQ ID NO: 2622.

The probes having the nucleotide sequence represented by SEQ ID NOS: 34938, 34939, 34940, 34941, 34942, 34943, 34944, 34945, 34946, 34947, 34948 were selected from the ORFs having the nucleotide sequence represented by SEQ ID NO: 2623.

The probes having the nucleotide sequence represented by SEQ ID NOS: 34949, 34950, 34951, 34952, 34953, 34954, 34955, 34956, 34957, 34958, 34959 were selected from the ORFs having the nucleotide sequence represented by SEQ ID NO: 2624.

The probes having the nucleotide sequence represented by SEQ ID NOS: 34960, 34961, 34962, 34963, 34964, 34965, 34966, 34967, 34968, 34969, 34970 were selected from the ORFs having the nucleotide sequence represented by SEQ ID NO: 2625.

The probes having the nucleotide sequence represented by SEQ ID NOS: 34971, 34972, 34973, 34974, 34975, 34976, 34977, 34978, 34979, 34980, 34981 were selected from the ORFs having the nucleotide sequence represented by SEQ ID NO: 2626.

The probes having the nucleotide sequence represented by SEQ ID NOS: 34982, 34983, 34984, 34985, 34986, 34987, 34988, 34989, 34990, 34991, 34992 were selected from the ORFs having the nucleotide sequence represented by SEQ ID NO: 2627.

The probes having the nucleotide sequence represented by SEQ ID NOS: 34993, 34994, 34995, 34996, 34997, 34998, 34999, 35000, 35001, 35002, 35003 were selected from the ORFs having the nucleotide sequence represented by SEQ ID NO: 2628.

The probes having the nucleotide sequence represented by SEQ ID NOS: 35004, 35005, 35006, 35007, 35008, 35009, 35010, 35011, 35012, 35013, 35014 were selected from the ORFs having the nucleotide sequence represented by SEQ ID NO: 2629.

The probes having the nucleotide sequence represented by SEQ ID NOS: 35015, 35016, 35017, 35018, 35019, 35020, 35021, 35022, 35023, 35024, 35025 were selected from the ORFs having the nucleotide sequence represented by SEQ ID NO: 2630.

The probes having the nucleotide sequence represented by SEQ ID NOS: 35026, 35027, 35028, 35029, 35030, 35031, 35032, 35033, 35034, 35035, 35036 were selected from the ORFs having the nucleotide sequence represented by SEQ ID NO: 2631.

The probes having the nucleotide sequence represented by SEQ ID NOS: 35037, 35038, 35039, 35040, 35041, 35042, 35043, 35044, 35045, 35046, 35047 were selected from the ORFs having the nucleotide sequence represented by SEQ ID NO: 2632.

The probes having the nucleotide sequence represented by SEQ ID NOS: 35048, 35049, 35050, 35051, 35052, 35053, 35054, 35055, 35056, 35057, 35058 were selected from the ORFs having the nucleotide sequence represented by SEQ ID NO: 2633.

The probes having the nucleotide sequence represented by SEQ ID NOS: 35059, 35060, 35061, 35062, 35063, 35064, 35065, 35066, 35067, 35068, 35069 were selected from the ORFs having the nucleotide sequence represented by SEQ ID NO: 2634.

The probes having the nucleotide sequence represented by SEQ ID NOS: 35070, 35071, 35072, 35073, 35074, 35075, 35076, 35077, 35078, 35079, 35080 were selected from the ORFs having the nucleotide sequence represented by SEQ ID NO: 2635.

The probes having the nucleotide sequence represented by SEQ ID NOS: 35081, 35082, 35083, 35084, 35085, 35086, 35087, 35088, 35089, 35090, 35091 were selected from the ORFs having the nucleotide sequence represented by SEQ ID NO: 2636.

The probes having the nucleotide sequence represented by SEQ ID NOS: 35092, 35093, 35094, 35095, 35096, 35097, 35098, 35099, 35100, 35101, 35102 were selected from the ORFs having the nucleotide sequence represented by SEQ ID NO: 2637.

The probes having the nucleotide sequence represented by SEQ ID NOS: 35103, 35104, 35105, 35106, 35107, 35108, 35109, 35110, 35111, 35112, 35113 were selected from the ORFs having the nucleotide sequence represented by SEQ ID NO: 2638.

The probes having the nucleotide sequence represented by SEQ ID NOS: 35114, 35115, 35116, 35117, 35118, 35119, 35120, 35121, 35122, 35123, 35124 were selected from the ORFs having the nucleotide sequence represented by SEQ ID NO: 2639.

The probes having the nucleotide sequence represented by SEQ ID NOS: 35125, 35126, 35127, 35128, 35129, 35130, 35131, 35132, 35133, 35134, 35135 were selected from the ORFs having the nucleotide sequence represented by SEQ ID NO: 2640.

The probes having the nucleotide sequence represented by SEQ ID NOS: 35136, 35137, 35138, 35139, 35140, 35141, 35142, 35143, 35144, 35145, 35146 were selected from the ORFs having the nucleotide sequence represented by SEQ ID NO: 2641.

The probes having the nucleotide sequence represented by SEQ ID NOS: 35147, 35148, 35149, 35150, 35151, 35152, 35153, 35154, 35155, 35156, 35157 were selected from the ORFs having the nucleotide sequence represented by SEQ ID NO: 2642.

The probes having the nucleotide sequence represented by SEQ ID NOS: 35158, 35159, 35160, 35161, 35162, 35163, 35164, 35165, 35166, 35167, 35168 were selected from the ORFs having the nucleotide sequence represented by SEQ ID NO: 2643.

The probes having the nucleotide sequence represented by SEQ ID NOS: 35169, 35170, 35171, 35172, 35173, 35174, 35175, 35176, 35177, 35178, 35179 were selected from the ORFs having the nucleotide sequence represented by SEQ ID NO: 2644.

The probes having the nucleotide sequence represented by SEQ ID NOS: 35180, 35181, 35182, 35183, 35184, 35185, 35186, 35187, 35188, 35189, 35190 were selected from the ORFs having the nucleotide sequence represented by SEQ ID NO: 2645.

The probes having the nucleotide sequence represented by SEQ ID NOS: 35191, 35192, 35193, 35194, 35195, 35196, 35197, 35198, 35199, 35200, 35201 were selected from the ORFs having the nucleotide sequence represented by SEQ ID NO: 2646.

The probes having the nucleotide sequence represented by SEQ ID NOS: 35202, 35203, 35204, 35205, 35206, 35207, 35208, 35209, 35210, 35211, 35212 were selected from the ORFs having the nucleotide sequence represented by SEQ ID NO: 2647.

The probes having the nucleotide sequence represented by SEQ ID NOS: 35213, 35214, 35215, 35216, 35217, 35218, 35219, 35220, 35221, 35222, 35223 were selected from the ORFs having the nucleotide sequence represented by SEQ ID NO: 2648.

The probes having the nucleotide sequence represented by SEQ ID NOS: 35224, 35225, 35226, 35227, 35228, 35229, 35230, 35231, 35232, 35233, 35234 were selected from the ORFs having the nucleotide sequence represented by SEQ ID NO: 2649.

The probes having the nucleotide sequence represented by SEQ ID NOS: 35235, 35236, 35237, 35238, 35239, 35240, 35241, 35242, 35243, 35244, 35245 were selected from the ORFs having the nucleotide sequence represented by SEQ ID NO: 2650.

The probes having the nucleotide sequence represented by SEQ ID NOS: 35246, 35247, 35248, 35249, 35250, 35251, 35252, 35253, 35254, 35255, 35256 were selected from the ORFs having the nucleotide sequence represented by SEQ ID NO: 2651.

The probes having the nucleotide sequence represented by SEQ ID NOS: 35257, 35258, 35259, 35260, 35261, 35262, 35263, 35264, 35265, 35266, 35267 were selected from the ORFs having the nucleotide sequence represented by SEQ ID NO: 2652.

The probes having the nucleotide sequence represented by SEQ ID NOS: 35268, 35269, 35270, 35271, 35272, 35273, 35274, 35275, 35276, 35277, 35278 were selected from the ORFs having the nucleotide sequence represented by SEQ ID NO: 2653.

The probes having the nucleotide sequence represented by SEQ ID NOS: 35279, 35280, 35281, 35282, 35283, 35284, 35285, 35286, 35287, 35288, 35289 were selected from the ORFs having the nucleotide sequence represented by SEQ ID NO: 2654.

The probes having the nucleotide sequence represented by SEQ ID NOS: 35290, 35291, 35292, 35293, 35294, 35295, 35296, 35297, 35298, 35299, 35300 were selected from the ORFs having the nucleotide sequence represented by SEQ ID NO: 2655.

The probes having the nucleotide sequence represented by SEQ ID NOS: 35301, 35302, 35303, 35304, 35305, 35306, 35307, 35308, 35309, 35310, 35311 were selected from the ORFs having the nucleotide sequence represented by SEQ ID NO: 2656.

The probes having the nucleotide sequence represented by SEQ ID NOS: 35312, 35313, 35314, 35315, 35316, 35317, 35318, 35319, 35320, 35321, 35322 were selected from the ORFs having the nucleotide sequence represented by SEQ ID NO: 2657.

The probes having the nucleotide sequence represented by SEQ ID NOS: 35323, 35324, 35325, 35326, 35327, 35328, 35329, 35330, 35331, 35332, 35333 were selected from the ORFs having the nucleotide sequence represented by SEQ ID NO: 2658.

The probes having the nucleotide sequence represented by SEQ ID NOS: 35334, 35335, 35336, 35337, 35338, 35339, 35340, 35341, 35342, 35343, 35344 were selected from the ORFs having the nucleotide sequence represented by SEQ ID NO: 2659.

The probes having the nucleotide sequence represented by SEQ ID NOS: 35345, 35346, 35347, 35348, 35349, 35350, 35351, 35352, 35353, 35354, 35355 were selected from the ORFs having the nucleotide sequence represented by SEQ ID NO: 2660.

The probes having the nucleotide sequence represented by SEQ ID NOS: 35356, 35357, 35358, 35359, 35360, 35361, 35362, 35363, 35364, 35365, 35366were selected from the ORFs having the nucleotide sequence represented by SEQ ID NO: 2661.

The probes having the nucleotide sequence represented by SEQ ID NOS: 35367, 35368, 35369, 35370, 35371, 35372, 35373, 35374, 35375, 35376, 35377 were selected from the ORFs having the nucleotide sequence represented by SEQ ID NO: 2662.

The probes having the nucleotide sequence represented by SEQ ID NOS: 35378, 35379, 35380, 35381, 35382, 35383, 35384, 35385, 35386, 35387, 35388 were selected from the ORFs having the nucleotide sequence represented by SEQ ID NO: 2663.

The probes having the nucleotide sequence represented by SEQ ID NOS: 35389, 35390, 35391, 35392, 35393, 35394, 35395, 35396, 35397, 35398, 35399 were selected from the ORFs having the nucleotide sequence represented by SEQ ID NO: 2664.

The probes having the nucleotide sequence represented by SEQ ID NOS: 35400, 35401, 35402, 35403, 35404, 35405, 35406, 35407, 35408, 35409, 35410 were selected from the ORFs having the nucleotide sequence represented by SEQ ID NO: 2665.

The probes having the nucleotide sequence represented by SEQ ID NOS: 35411, 35412, 35413, 35414, 35415, 35416, 35417, 35418, 35419, 35420, 35421 were selected from the ORFs having the nucleotide sequence represented by SEQ ID NO: 2666.

The probes having the nucleotide sequence represented by SEQ ID NOS: 35422, 35423, 35424, 35425, 35426, 35427, 35428, 35429, 35430, 35431, 35432 were selected from the ORFs having the nucleotide sequence represented by SEQ ID NO: 2667.

The probes having the nucleotide sequence represented by SEQ ID NOS: 35433, 35434, 35435, 35436, 35437, 35438, 35439, 35440, 35441, 35442, 35443 were selected from the ORFs having the nucleotide sequence represented by SEQ ID NO: 2668.

The probes having the nucleotide sequence represented by SEQ ID NOS: 35444, 35445, 35446, 35447, 35448, 35449, 35450, 35451, 35452, 35453, 35454 were selected from the ORFs having the nucleotide sequence represented by SEQ ID NO: 2669.

The probes having the nucleotide sequence represented by SEQ ID NOS: 35455, 35456, 35457, 35458, 35459, 35460, 35461, 35462, 35463, 35464, 35465 were selected from the ORFs having the nucleotide sequence represented by SEQ ID NO: 2670.

The probes having the nucleotide sequence represented by SEQ ID NOS: 35466, 35467, 35468, 35469, 35470, 35471, 35472, 35473, 35474, 35475, 35476 were selected from the ORFs having the nucleotide sequence represented by SEQ ID NO: 2671.

The probes having the nucleotide sequence represented by SEQ ID NOS: 35477, 35478, 35479, 35480, 35481, 35482, 35483, 35484, 35485, 35486, 35487 were selected from the ORFs having the nucleotide sequence represented by SEQ ID NO: 2672.

The probes having the nucleotide sequence represented by SEQ ID NOS: 35488, 35489, 35490, 35491, 35492, 35493, 35494, 35495, 35496, 35497, 35498 were selected from the ORFs having the nucleotide sequence represented by SEQ ID NO: 2673.

The probes having the nucleotide sequence represented by SEQ ID NOS: 35499, 35500, 35501, 35502, 35503, 35504, 35505, 35506, 35507, 35508, 35509 were selected from the ORFs having the nucleotide sequence represented by SEQ ID NO: 2674.

The probes having the nucleotide sequence represented by SEQ ID NOS: 35510, 35511, 35512, 35513, 35514, 35515, 35516, 35517, 35518, 35519, 35520 were selected from the ORFs having the nucleotide sequence represented by SEQ ID NO: 2675.

The probes having the nucleotide sequence represented by SEQ ID NOS: 35521, 35522, 35523, 35524, 35525, 35526, 35527, 35528, 35529, 35530, 35531 were selected from the ORFs having the nucleotide sequence represented by SEQ ID NO: 2676.

The probes having the nucleotide sequence represented by SEQ ID NOS: 35532, 35533, 35534, 35535, 35536, 35537, 35538, 35539, 35540, 35541, 35542 were selected from the ORFs having the nucleotide sequence represented by SEQ ID NO: 2677.

The probes having the nucleotide sequence represented by SEQ ID NOS: 35543, 35544, 35545, 35546, 35547, 35548, 35549, 35550, 35551, 35552, 35553 were selected from the ORFs having the nucleotide sequence represented by SEQ ID NO: 2678.

The probes having the nucleotide sequence represented by SEQ ID NOS: 35554, 35555, 35556, 35557, 35558, 35559, 35560, 35561, 35562, 35563, 35564 were selected from the ORFs having the nucleotide sequence represented by SEQ ID NO: 2679.

The probes having the nucleotide sequence represented by SEQ ID NOS: 35565, 35566, 35567, 35568, 35569, 35570, 35571, 35572, 35573, 35574, 35575 were selected from the ORFs having the nucleotide sequence represented by SEQ ID NO: 2680.

The probes having the nucleotide sequence represented by SEQ ID NOS: 35576, 35577, 35578, 35579, 35580, 35581, 35582, 35583, 35584, 35585, 35586 were selected from the ORFs having the nucleotide sequence represented by SEQ ID NO: 2681.

The probes having the nucleotide sequence represented by SEQ ID NOS: 35587, 35588, 35589, 35590, 35591, 35592, 35593, 35594, 35595, 35596, 35597 were selected from the ORFs having the nucleotide sequence represented by SEQ ID NO: 2682.

The probes having the nucleotide sequence represented by SEQ ID NOS: 35598, 35599, 35600, 35601, 35602, 35603, 35604, 35605, 35606, 35607, 35608 were selected from the ORFs having the nucleotide sequence represented by SEQ ID NO: 2683.

The probes having the nucleotide sequence represented by SEQ ID NOS: 35609, 35610, 35611, 35612, 35613, 35614, 35615, 35616, 35617, 35618, 35619 were selected from the ORFs having the nucleotide sequence represented by SEQ ID NO: 2684.

The probes having the nucleotide sequence represented by SEQ ID NOS: 35620, 35621, 35622, 35623, 35624, 35625, 35626, 35627, 35628, 35629, 35630 were selected from the ORFs having the nucleotide sequence represented by SEQ ID NO: 2685.

The probes having the nucleotide sequence represented by SEQ ID NOS: 35631, 35632, 35633, 35634, 35635, 35636, 35637, 35638, 35639, 35640, 35641 were selected from the ORFs having the nucleotide sequence represented by SEQ ID NO: 2686.

The probes having the nucleotide sequence represented by SEQ ID NOS: 35642, 35643, 35644, 35645, 35646, 35647, 35648, 35649, 35650, 35651, 35652 were selected from the ORFs having the nucleotide sequence represented by SEQ ID NO: 2687.

The probes having the nucleotide sequence represented by SEQ ID NOS: 35653, 35654, 35655, 35656, 35657, 35658, 35659, 35660, 35661, 35662, 35663 were selected from the ORFs having the nucleotide sequence represented by SEQ ID NO: 2688.

The probes having the nucleotide sequence represented by SEQ ID NOS: 35664, 35665, 35666, 35667, 35668, 35669, 35670, 35671, 35672, 35673, 35674 were selected from the ORFs having the nucleotide sequence represented by SEQ ID NO: 2689.

The probes having the nucleotide sequence represented by SEQ ID NOS: 35675, 35676, 35677, 35678, 35679, 35680, 35681, 35682, 35683, 35684, 35685 were selected from the ORFs having the nucleotide sequence represented by SEQ ID NO: 2690.

The probes having the nucleotide sequence represented by SEQ ID NOS: 35686, 35687, 35688, 35689, 35690, 35691, 35692, 35693, 35694, 35695, 35696 were selected from the ORFs having the nucleotide sequence represented by SEQ ID NO: 2691.

The probes having the nucleotide sequence represented by SEQ ID NOS: 35697, 35698, 35699, 35700, 35701, 35702, 35703, 35704, 35705, 35706, 35707 were selected from the ORFs having the nucleotide sequence represented by SEQ ID NO: 2692.

The probes having the nucleotide sequence represented by SEQ ID NOS: 35708, 35709, 35710, 35711, 35712, 35713, 35714, 35715, 35716, 35717, 35718 were selected from the ORFs having the nucleotide sequence represented by SEQ ID NO: 2693.

The probes having the nucleotide sequence represented by SEQ ID NOS: 35719, 35720, 35721, 35722, 35723, 35724, 35725, 35726, 35727, 35728, 35729 were selected from the ORFs having the nucleotide sequence represented by SEQ ID NO: 2694.

The probes having the nucleotide sequence represented by SEQ ID NOS: 35730, 35731, 35732, 35733, 35734, 35735, 35736, 35737, 35738, 35739, 35740 were selected from the ORFs having the nucleotide sequence represented by SEQ ID NO: 2695.

The probes having the nucleotide sequence represented by SEQ ID NOS: 35741, 35742, 35743, 35744, 35745, 35746, 35747, 35748, 35749, 35750, 35751 were selected from the ORFs having the nucleotide sequence represented by SEQ ID NO: 2696.

The probes having the nucleotide sequence represented by SEQ ID NOS: 35752, 35753, 35754, 35755, 35756, 35757, 35758, 35759, 35760, 35761, 35762 were selected from the ORFs having the nucleotide sequence represented by SEQ ID NO: 2697.

The probes having the nucleotide sequence represented by SEQ ID NOS: 35763, 35764, 35765, 35766, 35767, 35768, 35769, 35770, 35771, 35772, 35773, 35774, 35775, 35776, 35777, 35778, 35779, 35780, 35781, 35782, 35783, 35784 were selected from the ORFs having the nucleotide sequence represented by SEQ ID NO: 2698.

The probes having the nucleotide sequence represented by SEQ ID NOS: 35785, 35786, 35787, 35788, 35789, 35790, 35791, 35792, 35793, 35794, 35795 were selected from the ORFs having the nucleotide sequence represented by SEQ ID NO: 2699.

The probes having the nucleotide sequence represented by SEQ ID NOS: 35796, 35797, 35798, 35799, 35800, 35801, 35802, 35803, 35804, 35805, 35806 were selected from the ORFs having the nucleotide sequence represented by SEQ ID NO: 2700.

The probes having the nucleotide sequence represented by SEQ ID NOS: 35807, 35808, 35809, 35810, 35811, 35812, 35813, 35814, 35815, 35816, 35817 were selected from the ORFs having the nucleotide sequence represented by SEQ ID NO: 2701.

The probes having the nucleotide sequence represented by SEQ ID NOS: 35818, 35819, 35820, 35821, 35822, 35823, 35824, 35825, 35826, 35827, 35828 were selected from the ORFs having the nucleotide sequence represented by SEQ ID NO: 2702.

The probes having the nucleotide sequence represented by SEQ ID NOS: 35829, 35830, 35831, 35832, 35833, 35834, 35835, 35836, 35837, 35838, 35839 were selected from the ORFs having the nucleotide sequence represented by SEQ ID NO: 2703.

The probes having the nucleotide sequence represented by SEQ ID NOS: 35840, 35841, 35842, 35843, 35844, 35845, 35846, 35847, 35848, 35849, 35850 were selected from the ORFs having the nucleotide sequence represented by SEQ ID NO: 2704.

The probes having the nucleotide sequence represented by SEQ ID NOS: 35851, 35852, 35853, 35854, 35855, 35856, 35857, 35858, 35859, 35860, 35861 were selected from the ORFs having the nucleotide sequence represented by SEQ ID NO: 2705.

The probes having the nucleotide sequence represented by SEQ ID NOS: 35862, 35863, 35864, 35865, 35866, 35867, 35868, 35869, 35870, 35871, 35872 were selected from the ORFs having the nucleotide sequence represented by SEQ ID NO: 2706.

The probes having the nucleotide sequence represented by SEQ ID NOS: 35873, 35874, 35875, 35876, 35877, 35878, 35879, 35880, 35881, 35882, 35883 were selected from the ORFs having the nucleotide sequence represented by SEQ ID NO: 2707.

The probes having the nucleotide sequence represented by SEQ ID NOS: 35884, 35885, 35886, 35887, 35888, 35889, 35890, 35891, 35892, 35893, 35894 were selected from the ORFs having the nucleotide sequence represented by SEQ ID NO: 2708.

The probes having the nucleotide sequence represented by SEQ ID NOS: 35895, 35896, 35897, 35898, 35899, 35900, 35901, 35902, 35903, 35904, 35905 were selected from the ORFs having the nucleotide sequence represented by SEQ ID NO: 2709.

The probes having the nucleotide sequence represented by SEQ ID NOS: 35906, 35907, 35908, 35909, 35910, 35911, 35912, 35913, 35914, 35915, 35916 were selected from the ORFs having the nucleotide sequence represented by SEQ ID NO: 2710.

The probes having the nucleotide sequence represented by SEQ ID NOS: 35917, 35918, 35919, 35920, 35921, 35922, 35923, 35924, 35925, 35926, 35927 were selected from the ORFs having the nucleotide sequence represented by SEQ ID NO: 2711.

The probes having the nucleotide sequence represented by SEQ ID NOS: 35928, 35929, 35930, 35931, 35932, 35933, 35934, 35935, 35936, 35937, 35938 were selected from the ORFs having the nucleotide sequence represented by SEQ ID NO: 2712.

The probes having the nucleotide sequence represented by SEQ ID NOS: 35939, 35940, 35941, 35942, 35943, 35944, 35945, 35946, 35947, 35948, 35949 were selected from the ORFs having the nucleotide sequence represented by SEQ ID NO: 2713.

The probes having the nucleotide sequence represented by SEQ ID NOS: 35950, 35951, 35952, 35953, 35954, 35955, 35956, 35957, 35958, 35959, 35960 were selected from the ORFs having the nucleotide sequence represented by SEQ ID NO: 2714.

The probes having the nucleotide sequence represented by SEQ ID NOS: 35961, 35962, 35963, 35964, 35965, 35966, 35967, 35968, 35969, 35970, 35971 were selected from the ORFs having the nucleotide sequence represented by SEQ ID NO: 2715.

The probes having the nucleotide sequence represented by SEQ ID NOS: 35972, 35973, 35974, 35975, 35976, 35977, 35978, 35979, 35980, 35981, 35982 were selected from the ORFs having the nucleotide sequence represented by SEQ ID NO: 2716.

The probes having the nucleotide sequence represented by SEQ ID NOS: 35983, 35984, 35985, 35986, 35987, 35988, 35989, 35990, 35991, 35992, 35993 were selected from the ORFs having the nucleotide sequence represented by SEQ ID NO: 2717.

The probes having the nucleotide sequence represented by SEQ ID NOS: 35994, 35995, 35996, 35997, 35998, 35999, 36000, 36001, 36002, 36003, 36004 were selected from the ORFs having the nucleotide sequence represented by SEQ ID NO: 2718.

The probes having the nucleotide sequence represented by SEQ ID NOS: 36005, 36006, 36007, 36008, 36009, 36010, 36011, 36012, 36013, 36014, 36015 were selected from the ORFs having the nucleotide sequence represented by SEQ ID NO: 2719.

The probes having the nucleotide sequence represented by SEQ ID NOS: 36016, 36017, 36018, 36019, 36020, 36021, 36022, 36023, 36024, 36025, 36026 were selected from the ORFs having the nucleotide sequence represented by SEQ ID NO: 2720.

The probes having the nucleotide sequence represented by SEQ ID NOS: 36027, 36028, 36029, 36030, 36031, 36032, 36033, 36034, 36035, 36036, 36037 were selected from the ORFs having the nucleotide sequence represented by SEQ ID NO: 2721.

The probes having the nucleotide sequence represented by SEQ ID NOS: 36038, 36039, 36040, 36041, 36042, 36043, 36044, 36045, 36046, 36047, 36048 were selected from the ORFs having the nucleotide sequence represented by SEQ ID NO: 2722.

The probes having the nucleotide sequence represented by SEQ ID NOS: 36049, 36050, 36051, 36052, 36053, 36054, 36055, 36056, 36057, 36058, 36059 were selected from the ORFs having the nucleotide sequence represented by SEQ ID NO: 2723.

The probes having the nucleotide sequence represented by SEQ ID NOS: 36060, 36061, 36062, 36063, 36064, 36065, 36066, 36067, 36068, 36069, 36070 were selected from the ORFs having the nucleotide sequence represented by SEQ ID NO: 2724.

The probes having the nucleotide sequence represented by SEQ ID NOS: 36071, 36072, 36073, 36074, 36075, 36076, 36077, 36078, 36079, 36080, 36081 were selected from the ORFs having the nucleotide sequence represented by SEQ ID NO: 2725.

The probes having the nucleotide sequence represented by SEQ ID NOS: 36082, 36083, 36084, 36085, 36086, 36087, 36088, 36089, 36090, 36091, 36092 were selected from the ORFs having the nucleotide sequence represented by SEQ ID NO: 2726.

The probes having the nucleotide sequence represented by SEQ ID NOS: 36093, 36094, 36095, 36096, 36097, 36098, 36099, 36100, 36101, 36102, 36103 were selected from the ORFs having the nucleotide sequence represented by SEQ ID NO: 2727.

The probes having the nucleotide sequence represented by SEQ ID NOS: 36104, 36105, 36106, 36107, 36108, 36109, 36110, 36111, 36112, 36113, 36114 were selected from the ORFs having the nucleotide sequence represented by SEQ ID NO: 2728.

The probes having the nucleotide sequence represented by SEQ ID NOS: 36115, 36116, 36117, 36118, 36119, 36120, 36121, 36122, 36123, 36124, 36125 were selected from the ORFs having the nucleotide sequence represented by SEQ ID NO: 2729.

The probes having the nucleotide sequence represented by SEQ ID NOS: 36126, 36127, 36128, 36129, 36130, 36131, 36132, 36133, 36134, 36135, 36136 were selected from the ORFs having the nucleotide sequence represented by SEQ ID NO: 2730.

The probes having the nucleotide sequence represented by SEQ ID NOS: 36137, 36138, 36139, 36140, 36141, 36142, 36143, 36144, 36145, 36146, 36147 were selected from the ORFs having the nucleotide sequence represented by SEQ ID NO: 2731.

The probes having the nucleotide sequence represented by SEQ ID NOS: 36148, 36149, 36150, 36151, 36152, 36153, 36154, 36155, 36156, 36157, 36158 were selected from the ORFs having the nucleotide sequence represented by SEQ ID NO: 2732.

The probes having the nucleotide sequence represented by SEQ ID NOS: 36159, 36160, 36161, 36162, 36163, 36164, 36165, 36166, 36167, 36168, 36169 were selected from the ORFs having the nucleotide sequence represented by SEQ ID NO: 2733.

The probes having the nucleotide sequence represented by SEQ ID NOS: 36170, 36171, 36172, 36173, 36174, 36175, 36176, 36177, 36178, 36179, 36180 were selected from the ORFs having the nucleotide sequence represented by SEQ ID NO: 2734.

The probes having the nucleotide sequence represented by SEQ ID NOS: 36181, 36182, 36183, 36184, 36185, 36186, 36187, 36188, 36189, 36190, 36191 were selected from the ORFs having the nucleotide sequence represented by SEQ ID NO: 2735.

The probes having the nucleotide sequence represented by SEQ ID NOS: 36192, 36193, 36194, 36195, 36196, 36197, 36198, 36199, 36200, 36201, 36202 were selected from the ORFs having the nucleotide sequence represented by SEQ ID NO: 2736.

The probes having the nucleotide sequence represented by SEQ ID NOS: 36203, 36204, 36205, 36206, 36207, 36208, 36209, 36210, 36211, 36212, 36213 were selected from the ORFs having the nucleotide sequence represented by SEQ ID NO: 2737.

The probes having the nucleotide sequence represented by SEQ ID NOS: 36214, 36215, 36216, 36217, 36218, 36219, 36220, 36221, 36222, 36223, 36224 were selected from the ORFs having the nucleotide sequence represented by SEQ ID NO: 2738.

The probes having the nucleotide sequence represented by SEQ ID NOS: 36225, 36226, 36227, 36228, 36229, 36230, 36231, 36232, 36233, 36234, 36235 were selected from the ORFs having the nucleotide sequence represented by SEQ ID NO: 2739.

The probes having the nucleotide sequence represented by SEQ ID NOS: 36236, 36237, 36238, 36239, 36240, 36241, 36242, 36243, 36244, 36245, 36246 were selected from the ORFs having the nucleotide sequence represented by SEQ ID NO: 2740.

The probes having the nucleotide sequence represented by SEQ ID NOS: 36247, 36248, 36249, 36250, 36251, 36252, 36253, 36254, 36255, 36256, 36257 were selected from the ORFs having the nucleotide sequence represented by SEQ ID NO: 2741.

The probes having the nucleotide sequence represented by SEQ ID NOS: 36258, 36259, 36260, 36261, 36262, 36263, 36264, 36265, 36266, 36267, 36268 were selected from the ORFs having the nucleotide sequence represented by SEQ ID NO: 2742.

The probes having the nucleotide sequence represented by SEQ ID NOS: 36269, 36270, 36271, 36272, 36273, 36274, 36275, 36276, 36277, 36278, 36279 were selected from the ORFs having the nucleotide sequence represented by SEQ ID NO: 2743.

The probes having the nucleotide sequence represented by SEQ ID NOS: 36280, 36281, 36282, 36283, 36284, 36285, 36286, 36287, 36288, 36289, 36290 were selected from the ORFs having the nucleotide sequence represented by SEQ ID NO: 2744.

The probes having the nucleotide sequence represented by SEQ ID NOS: 36291, 36292, 36293, 36294, 36295, 36296, 36297, 36298, 36299, 36300, 36301 were selected from the ORFs having the nucleotide sequence represented by SEQ ID NO: 2745.

The probes having the nucleotide sequence represented by SEQ ID NOS: 36302, 36303, 36304, 36305, 36306, 36307, 36308, 36309, 36310, 36311, 36312 were selected from the ORFs having the nucleotide sequence represented by SEQ ID NO: 2746.

The probes having the nucleotide sequence represented by SEQ ID NOS: 36313, 36314, 36315, 36316, 36317, 36318, 36319, 36320, 36321, 36322, 36323 were selected from the ORFs having the nucleotide sequence represented by SEQ ID NO: 2747.

The probes having the nucleotide sequence represented by SEQ ID NOS: 36324, 36325, 36326, 36327, 36328, 36329, 36330, 36331, 36332, 36333, 36334 were selected from the ORFs having the nucleotide sequence represented by SEQ ID NO: 2748.

The probes having the nucleotide sequence represented by SEQ ID NOS: 36335, 36336, 36337, 36338, 36339, 36340, 36341, 36342, 36343, 36344, 36345 were selected from the ORFs having the nucleotide sequence represented by SEQ ID NO: 2749.

The probes having the nucleotide sequence represented by SEQ ID NOS: 36346, 36347, 36348, 36349, 36350, 36351, 36352, 36353, 36354, 36355, 36356 were selected from the ORFs having the nucleotide sequence represented by SEQ ID NO: 2750.

The probes having the nucleotide sequence represented by SEQ ID NOS: 36357, 36358, 36359, 36360, 36361, 36362, 36363, 36364, 36365, 36366, 36367 were selected from the ORFs having the nucleotide sequence represented by SEQ ID NO: 2751.

The probes having the nucleotide sequence represented by SEQ ID NOS: 36368, 36369, 36370, 36371, 36372, 36373, 36374, 36375, 36376, 36377, 36378 were selected from the ORFs having the nucleotide sequence represented by SEQ ID NO: 2752.

The probes having the nucleotide sequence represented by SEQ ID NOS: 36379, 36380, 36381, 36382, 36383, 36384, 36385, 36386, 36387, 36388, 36389 were selected from the ORFs having the nucleotide sequence represented by SEQ ID NO: 2753.

The probes having the nucleotide sequence represented by SEQ ID NOS: 36390, 36391, 36392, 36393, 36394, 36395, 36396, 36397, 36398, 36399, 36400 were selected from the ORFs having the nucleotide sequence represented by SEQ ID NO: 2754.

The probes having the nucleotide sequence represented by SEQ ID NOS: 36401, 36402, 36403, 36404, 36405, 36406, 36407, 36408, 36409, 36410, 36411 were selected from the ORFs having the nucleotide sequence represented by SEQ ID NO: 2755.

The probes having the nucleotide sequence represented by SEQ ID NOS: 36412, 36413, 36414, 36415, 36416, 36417, 36418, 36419, 36420, 36421, 36422 were selected from the ORFs having the nucleotide sequence represented by SEQ ID NO: 2756.

The probes having the nucleotide sequence represented by SEQ ID NOS: 36423, 36424, 36425, 36426, 36427, 36428, 36429, 36430, 36431, 36432, 36433 were selected from the ORFs having the nucleotide sequence represented by SEQ ID NO: 2757.

The probes having the nucleotide sequence represented by SEQ ID NOS: 36434, 36435, 36436, 36437, 36438, 36439, 36440, 36441, 36442, 36443, 36444 were selected from the ORFs having the nucleotide sequence represented by SEQ ID NO: 2758.

The probes having the nucleotide sequence represented by SEQ ID NOS: 36445, 36446, 36447, 36448, 36449, 36450, 36451, 36452, 36453, 36454, 36455 were selected from the ORFs having the nucleotide sequence represented by SEQ ID NO: 2759.

The probes having the nucleotide sequence represented by SEQ ID NOS: 36456, 36457, 36458, 36459, 36460, 36461, 36462, 36463, 36464, 36465, 36466 were selected from the ORFs having the nucleotide sequence represented by SEQ ID NO: 2760.

The probes having the nucleotide sequence represented by SEQ ID NOS: 36467, 36468, 36469, 36470, 36471, 36472, 36473, 36474, 36475, 36476, 36477 were selected from the ORFs having the nucleotide sequence represented by SEQ ID NO: 2761.

The probes having the nucleotide sequence represented by SEQ ID NOS: 36478, 36479, 36480, 36481, 36482, 36483, 36484, 36485, 36486, 36487, 36488 were selected from the ORFs having the nucleotide sequence represented by SEQ ID NO: 2762.

The probes having the nucleotide sequence represented by SEQ ID NOS: 36489, 36490, 36491, 36492, 36493, 36494, 36495, 36496, 36497, 36498, 36499 were selected from the ORFs having the nucleotide sequence represented by SEQ ID NO: 2763.

The probes having the nucleotide sequence represented by SEQ ID NOS: 36500, 36501, 36502, 36503, 36504, 36505, 36506, 36507, 36508, 36509, 36510 were selected from the ORFs having the nucleotide sequence represented by SEQ ID NO: 2764.

The probes having the nucleotide sequence represented by SEQ ID NOS: 36511, 36512, 36513, 36514, 36515, 36516, 36517, 36518, 36519, 36520, 36521 were selected from the ORFs having the nucleotide sequence represented by SEQ ID NO: 2765.

The probes having the nucleotide sequence represented by SEQ ID NOS: 36522, 36523, 36524, 36525, 36526, 36527, 36528, 36529, 36530, 36531, 36532 were selected from the ORFs having the nucleotide sequence represented by SEQ ID NO: 2766.

The probes having the nucleotide sequence represented by SEQ ID NOS: 36533, 36534, 36535, 36536, 36537, 36538, 36539, 36540, 36541, 36542, 36543 were selected from the ORFs having the nucleotide sequence represented by SEQ ID NO: 2767.

The probes having the nucleotide sequence represented by SEQ ID NOS: 36544, 36545, 36546, 36547, 36548, 36549, 36550, 36551, 36552, 36553, 36554 were selected from the ORFs having the nucleotide sequence represented by SEQ ID NO: 2768.

The probes having the nucleotide sequence represented by SEQ ID NOS: 36555, 36556, 36557, 36558, 36559, 36560, 36561, 36562, 36563, 36564, 36565 were selected from the ORFs having the nucleotide sequence represented by SEQ ID NO: 2769.

The probes having the nucleotide sequence represented by SEQ ID NOS: 36566, 36567, 36568, 36569, 36570, 36571, 36572, 36573, 36574, 36575, 36576 were selected from the ORFs having the nucleotide sequence represented by SEQ ID NO: 2770.

The probes having the nucleotide sequence represented by SEQ ID NOS: 36577, 36578, 36579, 36580, 36581, 36582, 36583, 36584, 36585, 36586, 36587 were selected from the ORFs having the nucleotide sequence represented by SEQ ID NO: 2771.

The probes having the nucleotide sequence represented by SEQ ID NOS: 36588, 36589, 36590, 36591, 36592, 36593, 36594, 36595, 36596, 36597, 36598 were selected from the ORFs having the nucleotide sequence represented by SEQ ID NO: 2772.

The probes having the nucleotide sequence represented by SEQ ID NOS: 36599, 36600, 36601, 36602, 36603, 36604, 36605, 36606, 36607, 36608, 36609 were selected from the ORFs having the nucleotide sequence represented by SEQ ID NO: 2773.

The probes having the nucleotide sequence represented by SEQ ID NOS: 36610, 36611, 36612, 36613, 36614, 36615, 36616, 36617, 36618, 36619, 36620 were selected from the ORFs having the nucleotide sequence represented by SEQ ID NO: 2774.

The probes having the nucleotide sequence represented by SEQ ID NOS: 36621, 36622, 36623, 36624, 36625, 36626, 36627, 36628, 36629, 36630, 36631 were selected from the ORFs having the nucleotide sequence represented by SEQ ID NO: 2775.

The probes having the nucleotide sequence represented by SEQ ID NOS: 36632, 36633, 36634, 36635, 36636, 36637, 36638, 36639, 36640, 36641, 36642 were selected from the ORFs having the nucleotide sequence represented by SEQ ID NO: 2776.

The probes having the nucleotide sequence represented by SEQ ID NOS: 36643, 36644, 36645, 36646, 36647, 36648, 36649, 36650, 36651, 36652, 36653 were selected from the ORFs having the nucleotide sequence represented by SEQ ID NO: 2777.

The probes having the nucleotide sequence represented by SEQ ID NOS: 36654, 36655, 36656, 36657, 36658, 36659, 36660, 36661, 36662, 36663, 36664 were selected from the ORFs having the nucleotide sequence represented by SEQ ID NO: 2778.

The probes having the nucleotide sequence represented by SEQ ID NOS: 36665, 36666, 36667, 36668, 36669, 36670, 36671, 36672, 36673, 36674, 36675 were selected from the ORFs having the nucleotide sequence represented by SEQ ID NO: 2779.

The probes having the nucleotide sequence represented by SEQ ID NOS: 36676, 36677, 36678, 36679, 36680, 36681, 36682, 36683, 36684, 36685, 36686 were selected from the ORFs having the nucleotide sequence represented by SEQ ID NO: 2780.

The probes having the nucleotide sequence represented by SEQ ID NOS: 36687, 36688, 36689, 36690, 36691, 36692, 36693, 36694, 36695, 36696, 36697 were selected from the ORFs having the nucleotide sequence represented by SEQ ID NO: 2781.

The probes having the nucleotide sequence represented by SEQ ID NOS: 36698, 36699, 36700, 36701, 36702, 36703, 36704, 36705, 36706, 36707, 36708 were selected from the ORFs having the nucleotide sequence represented by SEQ ID NO: 2782.

The probes having the nucleotide sequence represented by SEQ ID NOS: 36709, 36710, 36711, 36712, 36713, 36714, 36715, 36716, 36717, 36718, 36719 were selected from the ORFs having the nucleotide sequence represented by SEQ ID NO: 2783.

The probes having the nucleotide sequence represented by SEQ ID NOS: 36720, 36721, 36722, 36723, 36724, 36725, 36726, 36727, 36728, 36729, 36730 were selected from the ORFs having the nucleotide sequence represented by SEQ ID NO: 2784.

The probes having the nucleotide sequence represented by SEQ ID NOS: 36731, 36732, 36733, 36734, 36735, 36736, 36737, 36738, 36739, 36740, 36741 were selected from the ORFs having the nucleotide sequence represented by SEQ ID NO: 2785.

The probes having the nucleotide sequence represented by SEQ ID NOS: 36742, 36743, 36744, 36745, 36746, 36747, 36748, 36749, 36750, 36751, 36752 were,selected from the ORFs having the nucleotide sequence represented by SEQ ID NO: 2786.

The probes having the nucleotide sequence represented by SEQ ID NOS: 36753, 36754, 36755, 36756, 36757, 36758, 36759, 36760, 36761, 36762, 36763 were selected from the ORFs having the nucleotide sequence represented by SEQ ID NO: 2787.

The probes having the nucleotide sequence represented by SEQ ID NOS: 36764, 36765, 36766, 36767, 36768, 36769, 36770, 36771, 36772, 36773, 36774 were selected from the ORFs having the nucleotide sequence represented by SEQ ID NO: 2788.

The probes having the nucleotide sequence represented by SEQ ID NOS: 36775, 36776, 36777, 36778, 36779, 36780, 36781, 36782, 36783, 36784, 36785 were selected from the ORFs having the nucleotide sequence represented by SEQ ID NO: 2789.

The probes having the nucleotide sequence represented by SEQ ID NOS: 36786, 36787, 36788, 36789, 36790, 36791, 36792, 36793, 36794, 36795, 36796 were selected from the ORFs having the nucleotide sequence represented by SEQ ID NO: 2790.

The probes having the nucleotide sequence represented by SEQ ID NOS: 36797, 36798, 36799, 36800, 36801, 36802, 36803, 36804, 36805, 36806, 36807 were selected from the ORFs having the nucleotide sequence represented by SEQ ID NO: 2791.

The probes having the nucleotide sequence represented by SEQ ID NOS: 36808, 36809, 36810, 36811, 36812, 36813, 36814, 36815, 36816, 36817, 36818 were selected from the ORFs having the nucleotide sequence represented by SEQ ID NO: 2792.

The probes having the nucleotide sequence represented by SEQ ID NOS: 36819, 36820, 36821, 36822, 36823, 36824, 36825, 36826, 36827, 36828, 36829 were selected from the ORFs having the nucleotide sequence represented by SEQ ID NO: 2793.

The probes having the nucleotide sequence represented by SEQ ID NOS: 36830, 36831, 36832, 36833, 36834, 36835, 36836, 36837, 36838, 36839, 36840 were selected from the ORFs having the nucleotide sequence represented by SEQ ID NO: 2794.

The probes having the nucleotide sequence represented by SEQ ID NOS: 36841, 36842, 36843, 36844, 36845, 36846, 36847, 36848, 36849, 36850, 36851 were selected from the ORFs having the nucleotide sequence represented by SEQ ID NO: 2795.

The probes having the nucleotide sequence represented by SEQ ID NOS: 36852, 36853, 36854, 36855, 36856, 36857, 36858, 36859, 36860, 36861, 36862 were selected from the ORFs having the nucleotide sequence represented by SEQ ID NO: 2796.

The probes having the nucleotide sequence represented by SEQ ID NOS: 36863, 36864, 36865, 36866, 36867, 36868, 36869, 36870, 36871, 36872, 36873 were selected from the ORFs having the nucleotide sequence represented by SEQ ID NO: 2797.

The probes having the nucleotide sequence represented by SEQ ID NOS: 36874, 36875, 36876, 36877, 36878, 36879, 36880, 36881, 36882, 36883, 36884 were selected from the ORFs having the nucleotide sequence represented by SEQ ID NO: 2798.

The probes having the nucleotide sequence represented by SEQ ID NOS: 36885, 36886, 36887, 36888, 36889, 36890, 36891, 36892, 36893, 36894, 36895 were selected from the ORFs having the nucleotide sequence represented by SEQ ID NO: 2799.

The probes having the nucleotide sequence represented by SEQ ID NOS: 36896, 36897, 36898, 36899, 36900, 36901, 36902, 36903, 36904, 36905, 36906 were selected from the ORFs having the nucleotide sequence represented by SEQ ID NO: 2800.

The probes having the nucleotide sequence represented by SEQ ID NOS: 36907, 36908, 36909, 36910, 36911, 36912, 36913, 36914, 36915, 36916, 36917 were selected from the ORFs having the nucleotide sequence represented by SEQ ID NO: 2801.

The probes having the nucleotide sequence represented by SEQ ID NOS: 36918, 36919, 36920, 36921, 36922, 36923, 36924, 36925, 36926, 36927, 36928 were selected from the ORFs having the nucleotide sequence represented by SEQ ID NO: 2802.

The probes having the nucleotide sequence represented by SEQ ID NOS: 36929, 36930, 36931, 36932, 36933, 36934, 36935, 36936, 36937, 36938, 36939 were selected from the ORFs having the nucleotide sequence represented by SEQ ID NO: 2803.

The probes having the nucleotide sequence represented by SEQ ID NOS: 36940, 36941, 36942, 36943, 36944, 36945, 36946, 36947, 36948, 36949, 36950 were selected from the ORFs having the nucleotide sequence represented by SEQ ID NO: 2804.

The probes having the nucleotide sequence represented by SEQ ID NOS: 36951, 36952, 36953, 36954, 36955, 36956, 36957, 36958, 36959, 36960, 36961 were selected from the ORFs having the nucleotide sequence represented by SEQ ID NO: 2805.

The probes having the nucleotide sequence represented by SEQ ID NOS: 36962, 36963, 36964, 36965, 36966, 36967, 36968, 36969, 36970, 36971, 36972 were selected from the ORFs having the nucleotide sequence represented by SEQ ID NO: 2806.

The probes having the nucleotide sequence represented by SEQ ID NOS: 36973, 36974, 36975, 36976, 36977, 36978, 36979, 36980, 36981, 36982, 36983 were selected from the ORFs having the nucleotide sequence represented by SEQ ID NO: 2807.

The probes having the nucleotide sequence represented by SEQ ID NOS: 36984, 36985, 36986, 36987, 36988, 36989, 36990, 36991, 36992, 36993, 36994 were selected from the ORFs having the nucleotide sequence represented by SEQ ID NO: 2808.

The probes having the nucleotide sequence represented by SEQ ID NOS: 36995, 36996, 36997, 36998, 36999, 37000, 37001, 37002, 37003, 37004, 37005 were selected from the ORFs having the nucleotide sequence represented by SEQ ID NO: 2809.

The probes having the nucleotide sequence represented by SEQ ID NOS: 37006, 37007, 37008, 37009, 37010, 37011, 37012, 37013, 37014, 37015, 37016 were selected from the ORFs having the nucleotide sequence represented by SEQ ID NO: 2810.

The probes having the nucleotide sequence represented by SEQ ID NOS: 37017, 37018, 37019, 37020, 37021, 37022, 37023, 37024, 37025, 37026, 37027 were selected from the ORFs having the nucleotide sequence represented by SEQ ID NO: 2811.

The probes having the nucleotide sequence represented by SEQ ID NOS: 37028, 37029, 37030, 37031, 37032, 37033, 37034, 37035, 37036, 37037, 37038 were selected from the ORFs having the nucleotide sequence represented by SEQ ID NO: 2812.

The probes having the nucleotide sequence represented by SEQ ID NOS: 37039, 37040, 37041, 37042, 37043, 37044, 37045, 37046, 37047, 37048, 37049 were selected from the ORFs having the nucleotide sequence represented by SEQ ID NO: 2813.

The probes having the nucleotide sequence represented by SEQ ID NOS: 37050, 37051, 37052, 37053, 37054, 37055, 37056, 37057, 37058, 37059, 37060 were selected from the ORFs having the nucleotide sequence represented by SEQ ID NO: 2814.

The probes having the nucleotide sequence represented by SEQ ID NOS: 37061, 37062, 37063, 37064, 37065, 37066, 37067, 37068, 37069, 37070, 37071 were selected from the ORFs having the nucleotide sequence represented by SEQ ID NO: 2815.

The probes having the nucleotide sequence represented by SEQ ID NOS: 37072, 37073, 37074, 37075, 37076, 37077, 37078, 37079, 37080, 37081, 37082 were selected from the ORFs having the nucleotide sequence represented by SEQ ID NO: 2816.

The probes having the nucleotide sequence represented by SEQ ID NOS: 37083, 37084, 37085, 37086, 37087, 37088, 37089, 37090, 37091, 37092, 37093 were selected from the ORFs having the nucleotide sequence represented by SEQ ID NO: 2817.

The probes having the nucleotide sequence represented by SEQ ID NOS: 37094, 37095, 37096, 37097, 37098, 37099, 37100, 37101, 37102, 37103, 37104 were selected from the ORFs having the nucleotide sequence represented by SEQ ID NO: 2818.

The probes having the nucleotide sequence represented by SEQ ID NOS: 37105, 37106, 37107, 37108, 37109, 37110, 37111, 37112, 37113, 37114, 37115 were selected from the ORFs having the nucleotide sequence represented by SEQ ID NO: 2819.

The probes having the nucleotide sequence represented by SEQ ID NOS: 37116, 37117, 37118, 37119, 37120, 37121, 37122, 37123, 37124, 37125, 37126 were selected from the ORFs having the nucleotide sequence represented by SEQ ID NO: 2820.

The probes having the nucleotide sequence represented by SEQ ID NOS: 37127, 37128, 37129, 37130, 37131, 37132, 37133, 37134, 37135, 37136, 37137 were selected from the ORFs having the nucleotide sequence represented by SEQ ID NO: 2821.

The probes having the nucleotide sequence represented by SEQ ID NOS: 37138, 37139, 37140, 37141, 37142, 37143, 37144, 37145, 37146, 37147, 37148 were selected from the ORFs having the nucleotide sequence represented by SEQ ID NO: 2822.

The probes having the nucleotide sequence represented by SEQ ID NOS: 37149, 37150, 37151, 37152, 37153, 37154, 37155, 37156, 37157, 37158, 37159 were selected from the ORFs having the nucleotide sequence represented by SEQ ID NO: 2823.

The probes having the nucleotide sequence represented by SEQ ID NOS: 37160, 37161, 37162, 37163, 37164, 37165, 37166, 37167, 37168, 37169, 37170 were selected from the ORFs having the nucleotide sequence represented by SEQ ID NO: 2824.

The probes having the nucleotide sequence represented by SEQ ID NOS: 37171, 37172, 37173, 37174, 37175, 37176, 37177, 37178, 37179, 37180, 37181 were selected from the ORFs having the nucleotide sequence represented by SEQ ID NO: 2825.

The probes having the nucleotide sequence represented by SEQ ID NOS: 37182, 37183, 37184, 37185, 37186, 37187, 37188, 37189, 37190, 37191, 37192 were selected from the ORFs having the nucleotide sequence represented by SEQ ID NO: 2826.

The probes having the nucleotide sequence represented by SEQ ID NOS: 37193, 37194, 37195, 37196, 37197, 37198, 37199, 37200, 37201, 37202, 37203 were selected from the ORFs having the nucleotide sequence represented by SEQ ID NO: 2827.

The probes having the nucleotide sequence represented by SEQ ID NOS: 37204, 37205, 37206, 37207, 37208, 37209, 37210, 37211, 37212, 37213, 37214 were selected from the ORFs having the nucleotide sequence represented by SEQ ID NO: 2828.

The probes having the nucleotide sequence represented by SEQ ID NOS: 37215, 37216, 37217, 37218, 37219, 37220, 37221, 37222, 37223, 37224, 37225 were selected from the ORFs having the nucleotide sequence represented by SEQ ID NO: 2829.

The probes having the nucleotide sequence represented by SEQ ID NOS: 37226, 37227, 37228, 37229, 37230, 37231, 37232, 37233, 37234, 37235, 37236 were selected from the ORFs having the nucleotide sequence represented by SEQ ID NO: 2830.

The probes having the nucleotide sequence represented by SEQ ID NOS: 37237, 37238, 37239, 37240, 37241, 37242, 37243, 37244, 37245, 37246, 37247 were selected from the ORFs having the nucleotide sequence represented by SEQ ID NO: 2831.

The probes having the nucleotide sequence represented by SEQ ID NOS: 37248, 37249, 37250, 37251, 37252, 37253, 37254, 37255, 37256, 37257, 37258 were selected from the ORFs having the nucleotide sequence represented by SEQ ID NO: 2832.

The probes having the nucleotide sequence represented by SEQ ID NOS: 37259, 37260, 37261, 37262, 37263, 37264, 37265, 37266, 37267, 37268, 37269 were selected from the ORFs having the nucleotide sequence represented by SEQ ID NO: 2833.

The probes having the nucleotide sequence represented by SEQ ID NOS: 37270, 37271, 37272, 37273, 37274, 37275, 37276, 37277, 37278, 37279, 37280 were selected from the ORFs having the nucleotide sequence represented by SEQ ID NO: 2834.

The probes having the nucleotide sequence represented by SEQ ID NOS: 37281, 37282, 37283, 37284, 37285, 37286, 37287, 37288, 37289, 37290, 37291 were selected from the ORFs having the nucleotide sequence represented by SEQ ID NO: 2835.

The probes having the nucleotide sequence represented by SEQ ID NOS: 37292, 37293, 37294, 37295, 37296, 37297, 37298, 37299, 37300, 37301, 37302 were selected from the ORFs having the nucleotide sequence represented by SEQ ID NO: 2836.

The probes having the nucleotide sequence represented by SEQ ID NOS: 37303, 37304, 37305, 37306, 37307, 37308, 37309, 37310, 37311, 37312, 37313 were selected from the ORFs having the nucleotide sequence represented by SEQ ID NO: 2837.

The probes having the nucleotide sequence represented by SEQ ID NOS: 37314, 37315, 37316, 37317, 37318, 37319, 37320, 37321, 37322, 37323, 37324 were selected from the ORFs having the nucleotide sequence represented by SEQ ID NO: 2838.

The probes having the nucleotide sequence represented by SEQ ID NOS: 37325, 37326, 37327, 37328, 37329, 37330, 37331, 37332, 37333, 37334, 37335 were selected from the ORFs having the nucleotide sequence represented by SEQ ID NO: 2839.

The probes having the nucleotide sequence represented by SEQ ID NOS: 37336, 37337, 37338, 37339, 37340, 37341, 37342, 37343, 37344, 37345, 37346 were selected from the ORFs having the nucleotide sequence represented by SEQ ID NO: 2840.

The probes having the nucleotide sequence represented by SEQ ID NOS: 37347, 37348, 37349, 37350, 37351, 37352, 37353, 37354, 37355, 37356, 37357 were selected from the ORFs having the nucleotide sequence represented by SEQ ID NO: 2841.

The probes having the nucleotide sequence represented by SEQ ID NOS: 37358, 37359, 37360, 37361, 37362, 37363, 37364, 37365, 37366, 37367, 37368 were selected from the ORFs having the nucleotide sequence represented by SEQ ID NO: 2842.

The probes having the nucleotide sequence represented by SEQ ID NOS: 37369, 37370, 37371, 37372, 37373, 37374, 37375, 37376, 37377, 37378, 37379 were selected from the ORFs having the nucleotide sequence represented by SEQ ID NO: 2843.

The probes having the nucleotide sequence represented by SEQ ID NOS: 37380, 37381, 37382, 37383, 37384, 37385, 37386, 37387, 37388, 37389, 37390 were selected from the ORFs having the nucleotide sequence represented by SEQ ID NO: 2844.

The probes having the nucleotide sequence represented by SEQ ID NOS: 37391, 37392, 37393, 37394, 37395, 37396, 37397, 37398, 37399, 37400, 37401 were selected from the ORFs having the nucleotide sequence represented by SEQ ID NO: 2845.

The probes having the nucleotide sequence represented by SEQ ID NOS: 37402, 37403, 37404, 37405, 37406, 37407, 37408, 37409, 37410, 37411, 37412 were selected from the ORFs having the nucleotide sequence represented by SEQ ID NO: 2846.

The probes having the nucleotide sequence represented by SEQ ID NOS: 37413, 37414, 37415, 37416, 37417, 37418, 37419, 37420, 37421, 37422, 37423 were selected from the ORFs having the nucleotide sequence represented by SEQ ID NO: 2847.

The probes having the nucleotide sequence represented by SEQ ID NOS: 37424, 37425, 37426, 37427, 37428, 37429, 37430, 37431, 37432, 37433, 37434 were selected from the ORFs having the nucleotide sequence represented by SEQ ID NO: 2848.

The probes having the nucleotide sequence represented by SEQ ID NOS: 37435, 37436, 37437, 37438, 37439, 37440, 37441, 37442, 37443, 37444, 37445 were selected from the ORFs having the nucleotide sequence represented by SEQ ID NO: 2849.

The probes having the nucleotide sequence represented by SEQ ID NOS: 37446, 37447, 37448, 37449, 37450, 37451, 37452, 37453, 37454, 37455, 37456 were selected from the ORFs having the nucleotide sequence represented by SEQ ID NO: 2850.

The probes having the nucleotide sequence represented by SEQ ID NOS: 37457, 37458, 37459, 37460, 37461, 37462, 37463, 37464, 37465, 37466, 37467 were selected from the ORFs having the nucleotide sequence represented by SEQ ID NO: 2851.

The probes having the nucleotide sequence represented by SEQ ID NOS: 37468, 37469, 37470, 37471, 37472, 37473, 37474, 37475, 37476, 37477, 37478 were selected from the ORFs having the nucleotide sequence represented by SEQ ID NO: 2852.

The probes having the nucleotide sequence represented by SEQ ID NOS: 37479, 37480, 37481, 37482, 37483, 37484, 37485, 37486, 37487, 37488, 37489 were selected from the ORFs having the nucleotide sequence represented by SEQ ID NO: 2853.

The probes having the nucleotide sequence represented by SEQ ID NOS: 37490, 37491, 37492, 37493, 37494, 37495, 37496, 37497, 37498, 37499, 37500 were selected from the ORFs having the nucleotide sequence represented by SEQ ID NO: 2854.

The probes having the nucleotide sequence represented by SEQ ID NOS: 37501, 37502, 37503, 37504, 37505, 37506, 37507, 37508, 37509, 37510, 37511 were selected from the ORFs having the nucleotide sequence represented by SEQ ID NO: 2855.

The probes having the nucleotide sequence represented by SEQ ID NOS: 37512, 37513, 37514, 37515, 37516, 37517, 37518, 37519, 37520, 37521, 37522 were selected from the ORFs having the nucleotide sequence represented by SEQ ID. NO: 2856.

The probes having the nucleotide sequence represented by SEQ ID NOS: 37523, 37524, 37525, 37526, 37527, 37528, 37529, 37530, 37531, 37532, 37533 were selected from the ORFs having the nucleotide sequence represented by SEQ ID NO: 2857.

The probes having the nucleotide sequence represented by SEQ ID NOS: 37534, 37535, 37536, 37537, 37538, 37539, 37540, 37541, 37542, 37543, 37544 were selected from the ORFs having the nucleotide sequence represented by SEQ ID NO: 2858.

The probes having the nucleotide sequence represented by SEQ ID NOS: 37545, 37546, 37547, 37548, 37549, 37550, 37551, 37552, 37553, 37554, 37555 were selected from the ORFs having the nucleotide sequence represented by SEQ ID NO: 2859.

The probes having the nucleotide sequence represented by SEQ ID NOS: 37556, 37557, 37558, 37559, 37560, 37561, 37562, 37563, 37564, 37565, 37566 were selected from the ORFs having the nucleotide sequence represented by SEQ ID NO: 2860.

The probes having the nucleotide sequence represented by SEQ ID NOS: 37567, 37568, 37569, 37570, 37571, 37572, 37573, 37574, 37575, 37576, 37577 were selected from the ORFs having the nucleotide sequence represented by SEQ ID NO: 2861.

The probes having the nucleotide sequence represented by SEQ ID NOS: 37578, 37579, 37580, 37581, 37582, 37583, 37584, 37585, 37586, 37587, 37588 were selected from the ORFs having the nucleotide sequence represented by SEQ ID NO: 2862.

The probes having the nucleotide sequence represented by SEQ ID NOS: 37589, 37590, 37591, 37592, 37593, 37594, 37595, 37596, 37597, 37598, 37599 were selected from the ORFs having the nucleotide sequence represented by SEQ ID NO: 2863.

The probes having the nucleotide sequence represented by SEQ ID NOS: 37600, 37601, 37602, 37603, 37604, 37605, 37606, 37607, 37608, 37609, 37610 were selected from the ORFs having the nucleotide sequence represented by SEQ ID NO: 2864.

The probes having the nucleotide sequence represented by SEQ ID NOS: 37611, 37612, 37613, 37614, 37615, 37616, 37617, 37618, 37619, 37620, 37621 were selected from the ORFs having the nucleotide sequence represented by SEQ ID NO: 2865.

The probes having the nucleotide sequence represented by SEQ ID NOS: 37622, 37623, 37624, 37625, 37626, 37627, 37628, 37629, 37630, 37631, 37632 were selected from the ORFs having the nucleotide sequence represented by SEQ ID NO: 2866.

The probes having the nucleotide sequence represented by SEQ ID NOS: 37633, 37634, 37635, 37636, 37637, 37638, 37639, 37640, 37641, 37642, 37643 were selected from the ORFs having the nucleotide sequence represented by SEQ ID NO: 2867.

The probes having the nucleotide sequence represented by SEQ ID NOS: 37644, 37645, 37646, 37647, 37648, 37649, 37650, 37651, 37652, 37653, 37654 were selected from the ORFs having the nucleotide sequence represented by SEQ ID NO: 2868.

The probes having the nucleotide sequence represented by SEQ ID NOS: 37655, 37656, 37657, 37658, 37659, 37660, 37661, 37662, 37663, 37664, 37665 were selected from the ORFs having the nucleotide sequence represented by SEQ ID NO: 2869.

The probes having the nucleotide sequence represented by SEQ ID NOS: 37666, 37667, 37668, 37669, 37670, 37671, 37672, 37673, 37674, 37675, 37676 were selected from the ORFs having the nucleotide sequence represented by SEQ ID NO: 2870.

The probes having the nucleotide sequence represented by SEQ ID NOS: 37677, 37678, 37679, 37680, 37681, 37682, 37683, 37684, 37685, 37686, 37687 were selected from the ORFs having the nucleotide sequence represented by SEQ ID NO: 2871.

The probes having the nucleotide sequence represented by SEQ ID NOS: 37688, 37689, 37690, 37691, 37692, 37693, 37694, 37695, 37696, 37697, 37698 were selected from the ORFs having the nucleotide sequence represented by SEQ ID NO: 2872.

The probes having the nucleotide sequence represented by SEQ ID NOS: 37699, 37700, 37701, 37702, 37703, 37704, 37705, 37706, 37707, 37708, 37709 were selected from the ORFs having the nucleotide sequence represented by SEQ ID NO: 2873.

The probes having the nucleotide sequence represented by SEQ ID NOS: 37710, 37711, 37712, 37713, 37714, 37715, 37716, 37717, 37718, 37719, 37720 were selected from the ORFs having the nucleotide sequence represented by SEQ ID NO: 2874.

The probes having the nucleotide sequence represented by SEQ ID NOS: 37721, 37722, 37723, 37724, 37725, 37726, 37727, 37728, 37729, 37730, 37731 were selected from the ORFs having the nucleotide sequence represented by SEQ ID NO: 2875.

The probes having the nucleotide sequence represented by SEQ ID NOS: 37732, 37733, 37734, 37735, 37736, 37737, 37738, 37739, 37740, 37741, 37742 were selected from the ORFs having the nucleotide sequence represented by SEQ ID NO: 2876.

The probes having the nucleotide sequence represented by SEQ ID NOS: 37743, 37744, 37745, 37746, 37747, 37748, 37749, 37750, 37751, 37752, 37753 were selected from the ORFs having the nucleotide sequence represented by SEQ ID NO: 2877.

The probes having the nucleotide sequence represented by SEQ ID NOS: 37754, 37755, 37756, 37757, 37758, 37759, 37760, 37761, 37762, 37763, 37764 were selected from the ORFs having the nucleotide sequence represented by SEQ ID NO: 2878.

The probes having the nucleotide sequence represented by SEQ ID NOS: 37765, 37766, 37767, 37768, 37769, 37770, 37771, 37772, 37773, 37774, 37775 were selected from the ORFs having the nucleotide sequence represented by SEQ ID NO: 2879.

The probes having the nucleotide sequence represented by SEQ ID NOS: 37776, 37777, 37778, 37779, 37780, 37781, 37782, 37783, 37784, 37785, 37786 were selected from the ORFs having the nucleotide sequence represented by SEQ ID NO: 2880.

The probes having the nucleotide sequence represented by SEQ ID NOS: 37787, 37788, 37789, 37790, 37791, 37792, 37793, 37794, 37795, 37796, 37797 were selected from the ORFs having the nucleotide sequence represented by SEQ ID NO: 2881.

The probes having the nucleotide sequence represented by SEQ ID NOS: 37798, 37799, 37800, 37801, 37802, 37803, 37804, 37805, 37806, 37807, 37808 were selected from the ORFs having the nucleotide sequence represented by SEQ ID NO: 2882.

The probes having the nucleotide sequence represented by SEQ ID NOS: 37809, 37810, 37811, 37812, 37813, 37814, 37815, 37816, 37817, 37818, 37819 were selected from the ORFs having the nucleotide sequence represented by SEQ ID NO: 2883.

The probes having the nucleotide sequence represented by SEQ ID NOS: 37820, 37821, 37822, 37823, 37824, 37825, 37826, 37827, 37828, 37829, 37830 were selected from the ORFs having the nucleotide sequence represented by SEQ ID NO: 2884.

The probes having the nucleotide sequence represented by SEQ ID NOS: 37831, 37832, 37833, 37834, 37835, 37836, 37837, 37838, 37839, 37840, 37841 were selected from the ORFs having the nucleotide sequence represented by SEQ ID NO: 2885.

The probes having the nucleotide sequence represented by SEQ ID NOS: 37842, 37843, 37844, 37845, 37846, 37847, 37848, 37849, 37850, 37851, 37852 were selected from the ORFs having the nucleotide sequence represented by SEQ ID NO: 2886.

The probes having the nucleotide sequence represented by SEQ ID NOS: 37853, 37854, 37855, 37856, 37857, 37858, 37859, 37860, 37861, 37862, 37863 were selected from the ORFs having the nucleotide sequence represented by SEQ ID NO: 2887.

The probes having the nucleotide sequence represented by SEQ ID NOS: 37864, 37865, 37866, 37867, 37868, 37869, 37870, 37871, 37872, 37873, 37874 were selected from the ORFs having the nucleotide sequence represented by SEQ ID NO: 2888.

The probes having the nucleotide sequence represented by SEQ ID NOS: 37875, 37876, 37877, 37878, 37879, 37880, 37881, 37882, 37883, 37884, 37885 were selected from the ORFs having the nucleotide sequence represented by SEQ ID NO: 2889.

The probes having the nucleotide sequence represented by SEQ ID NOS: 37886, 37887, 37888, 37889, 37890, 37891, 37892, 37893, 37894, 37895, 37896 were selected from the ORFs having the nucleotide sequence represented by SEQ ID NO: 2890.

The probes having the nucleotide sequence represented by SEQ ID NOS: 37897, 37898, 37899, 37900, 37901, 37902, 37903, 37904, 37905, 37906, 37907 were selected from the ORFs having the nucleotide sequence represented by SEQ ID NO: 2891.

The probes having the nucleotide sequence represented by SEQ ID NOS: 37908, 37909, 37910, 37911, 37912, 37913, 37914, 37915, 37916, 37917, 37918 were selected from the ORFs having the nucleotide sequence represented by SEQ ID NO: 2892.

The probes having the nucleotide sequence represented by SEQ ID NOS: 37919, 37920, 37921, 37922, 37923, 37924, 37925, 37926, 37927, 37928, 37929 were selected from the ORFs having the nucleotide sequence represented by SEQ ID NO: 2893.

The probes having the nucleotide sequence represented by SEQ ID NOS: 37930, 37931, 37932, 37933, 37934, 37935, 37936, 37937, 37938, 37939, 37940 were selected from the ORFs having the nucleotide sequence represented by SEQ ID NO: 2894.

The probes having the nucleotide sequence represented by SEQ ID NOS: 37941, 37942, 37943, 37944, 37945, 37946, 37947, 37948, 37949, 37950, 37951 were selected from the ORFs having the nucleotide sequence represented by SEQ ID NO: 2895.

The probes having the nucleotide sequence represented by SEQ ID NOS: 37952, 37953, 37954, 37955, 37956, 37957, 37958, 37959, 37960, 37961, 37962 were selected from the ORFs having the nucleotide sequence represented by SEQ ID NO: 2896.

The probes having the nucleotide sequence represented by SEQ ID NOS: 37963, 37964, 37965, 37966, 37967, 37968, 37969, 37970, 37971, 37972, 37973, 37974, 37975, 37976, 37977, 37978, 37979, 37980, 37981, 37982, 37983, 37984 were selected from the ORFs having the nucleotide sequence represented by SEQ ID NO: 2897.

The probes having the nucleotide sequence represented by SEQ ID NOS: 37985, 37986, 37987, 37988, 37989, 37990, 37991, 37992, 37993, 37994, 37995 were selected from the ORFs having the nucleotide sequence represented by SEQ ID NO: 2898.

The probes having the nucleotide sequence represented by SEQ ID NOS: 37996, 37997, 37998, 37999, 38000, 38001, 38002, 38003, 38004, 38005, 38006 were selected from the ORFs having the nucleotide sequence represented by SEQ ID NO: 2899.

The probes having the nucleotide sequence represented by SEQ ID NOS: 38007, 38008, 38009, 38010, 38011, 38012, 38013, 38014, 38015, 38016, 38017 were selected from the ORFs having the nucleotide sequence represented by SEQ ID NO: 2900.

The probes having the nucleotide sequence represented by SEQ ID NOS: 38018, 38019, 38020, 38021, 38022, 38023, 38024, 38025, 38026, 38027, 38028 were selected from the ORFs having the nucleotide sequence represented by SEQ ID NO: 2901.

The probes having the nucleotide sequence represented by SEQ ID NOS: 38029, 38030, 38031, 38032, 38033, 38034, 38035, 38036, 38037, 38038, 38039 were selected from the ORFs having the nucleotide sequence represented by SEQ ID NO: 2902.

The probes having the nucleotide sequence represented by SEQ ID NOS: 38040, 38041, 38042, 38043, 38044, 38045, 38046, 38047, 38048, 38049, 38050 were selected from the ORFs having the nucleotide sequence represented by SEQ ID NO: 2903.

The probes having the nucleotide sequence represented by SEQ ID NOS: 38051, 38052, 38053, 38054, 38055, 38056, 38057, 38058, 38059, 38060, 38061 were selected from the ORFs having the nucleotide sequence represented by SEQ ID NO: 2904.

The probes having the nucleotide sequence represented by SEQ ID NOS: 38062, 38063, 38064, 38065, 38066, 38067, 38068, 38069, 38070, 38071, 38072 were selected from the ORFs having the nucleotide sequence represented by SEQ ID NO: 2905.

The probes having the nucleotide sequence represented by SEQ ID NOS: 38073, 38074, 38075, 38076, 38077, 38078, 38079, 38080, 38081, 38082, 38083 were selected from the ORFs having the nucleotide sequence represented by SEQ ID NO: 2906.

The probes having the nucleotide sequence represented by SEQ ID NOS: 38084, 38085, 38086, 38087, 38088, 38089, 38090, 38091, 38092, 38093, 38094 were selected from the ORFs having the nucleotide sequence represented by SEQ ID NO: 2907.

The probes having the nucleotide sequence represented by SEQ ID NOS: 38095, 38096, 38097, 38098, 38099, 38100, 38101, 38102, 38103, 38104, 38105 were selected from the ORFs having the nucleotide sequence represented by SEQ ID. NO: 2908.

The probes having the nucleotide sequence represented by SEQ ID NOS: 38106, 38107, 38108, 38109, 38110, 38111, 38112, 38113, 38114, 38115, 38116 were selected from the ORFs having the nucleotide sequence represented by SEQ ID NO: 2909.

The probes having the nucleotide sequence represented by SEQ ID NOS: 38117, 38118, 38119, 38120, 38121, 38122, 38123, 38124, 38125, 38126, 38127 were selected from the ORFs having the nucleotide sequence represented by SEQ ID NO: 2910.

The probes having the nucleotide sequence represented by SEQ ID NOS: 38128, 38129, 38130, 38131, 38132, 38133, 38134, 38135, 38136, 38137, 38138 were selected from the ORFs having the nucleotide sequence represented by SEQ ID NO: 2911.

The probes having the nucleotide sequence represented by SEQ ID NOS: 38139, 38140, 38141, 38142, 38143, 38144, 38145, 38146, 38147, 38148, 38149 were selected from the ORFs having the nucleotide sequence represented by SEQ ID NO: 2912.

The probes having the nucleotide sequence represented by SEQ ID NOS: 38150, 38151, 38152, 38153, 38154, 38155, 38156, 38157, 38158, 38159, 38160 were selected from the ORFs having the nucleotide sequence represented by SEQ ID NO: 2913.

The probes having the nucleotide sequence represented by SEQ ID NOS: 38161, 38162, 38163, 38164, 38165, 38166, 38167, 38168, 38169, 38170, 38171 were selected from the ORFs having the nucleotide sequence represented by SEQ ID NO: 2914.

The probes having the nucleotide sequence represented by SEQ ID NOS: 38172, 38173, 38174, 38175, 38176, 38177, 38178, 38179, 38180, 38181, 38182 were selected from the ORFs having the nucleotide sequence represented by SEQ ID NO: 2915.

The probes having the nucleotide sequence represented by SEQ ID NOS: 38183, 38184, 38185, 38186, 38187, 38188, 38189, 38190, 38191, 38192, 38193 were selected from the ORFs having the nucleotide sequence represented by SEQ ID NO: 2916.

The probes having the nucleotide sequence represented by SEQ ID NOS: 38194, 38195, 38196, 38197, 38198, 38199, 38200, 38201, 38202, 38203, 38204 were selected from the ORFs having the nucleotide sequence represented by SEQ ID NO: 2917.

The probes having the nucleotide sequence represented by SEQ ID NOS: 38205, 38206, 38207, 38208, 38209, 38210, 38211, 38212, 38213, 38214, 38215 were selected from the ORFs having the nucleotide sequence represented by SEQ ID NO: 2918.

The probes having the nucleotide sequence represented by SEQ ID NOS: 38216, 38217, 38218, 38219, 38220, 38221, 38222, 38223, 38224, 38225, 38226 were selected from the ORFs having the nucleotide sequence represented by SEQ ID NO: 2919.

The probes having the nucleotide sequence represented by SEQ ID NOS: 38227, 38228, 38229, 38230, 38231, 38232, 38233, 38234, 38235, 38236, 38237 were selected from the ORFs having the nucleotide sequence represented by SEQ ID NO: 2920.

The probes having the nucleotide sequence represented by SEQ ID NOS: 38238, 38239, 38240, 38241, 38242, 38243, 38244, 38245, 38246, 38247, 38248 were selected from the ORFs having the nucleotide sequence represented by SEQ ID NO: 2921.

The probes having the nucleotide sequence represented by SEQ ID NOS: 38249, 38250, 38251, 38252, 38253, 38254, 38255, 38256, 38257, 38258, 38259 were selected from the ORFs having the nucleotide sequence represented by SEQ ID NO: 2922.

The probes having the nucleotide sequence represented by SEQ ID NOS: 38260, 38261, 38262, 38263, 38264, 38265, 38266, 38267, 38268, 38269, 38270 were selected from the ORFs having the nucleotide sequence represented by SEQ ID NO: 2923.

The probes having the nucleotide sequence represented by SEQ ID NOS: 38271, 38272, 38273, 38274, 38275, 38276, 38277, 38278, 38279, 38280, 38281 were selected from the ORFs having the nucleotide sequence represented by SEQ ID NO: 2924.

The probes having the nucleotide sequence represented by SEQ ID NOS: 38282, 38283, 38284, 38285, 38286, 38287, 38288, 38289, 38290, 38291, 38292 were selected from the ORFs having the nucleotide sequence represented by SEQ ID NO: 2925.

The probes having the nucleotide sequence represented by SEQ ID NOS: 38293, 38294, 38295, 38296, 38297, 38298, 38299, 38300, 38301, 38302, 38303 were selected from the ORFs having the nucleotide sequence represented by SEQ ID NO: 2926.

The probes having the nucleotide sequence represented by SEQ ID NOS: 38304, 38305, 38306, 38307, 38308, 38309, 38310, 38311, 38312, 38313, 38314 were selected from the ORFs having the nucleotide sequence represented by SEQ ID NO: 2927.

The probes having the nucleotide sequence represented by SEQ ID NOS: 38315, 38316, 38317, 38318, 38319, 38320, 38321, 38322, 38323, 38324, 38325 were selected from the ORFs having the nucleotide sequence represented by SEQ ID NO: 2928.

The probes having the nucleotide sequence represented by SEQ ID NOS: 38326, 38327, 38328, 38329, 38330, 38331, 38332, 38333, 38334, 38335, 38336 were selected from the ORFs having the nucleotide sequence represented by SEQ ID NO: 2929.

The probes having the nucleotide sequence represented by SEQ ID NOS: 38337, 38338, 38339, 38340, 38341, 38342, 38343, 38344, 38345, 38346, 38347 were selected from the ORFs having the nucleotide sequence represented by SEQ ID NO: 2930.

The probes having the nucleotide sequence represented by SEQ ID NOS: 38348, 38349, 38350, 38351, 38352, 38353, 38354, 38355, 38356, 38357, 38358 were selected from the ORFs having the nucleotide sequence represented by SEQ ID NO: 2931.

The probes having the nucleotide sequence represented by SEQ ID NOS: 38359, 38360, 38361, 38362, 38363, 38364, 38365, 38366, 38367, 38368, 38369 were selected from the ORFs having the nucleotide sequence represented by SEQ ID NO: 2932.

The probes having the nucleotide sequence represented by SEQ ID NOS: 38370, 38371, 38372, 38373, 38374, 38375, 38376, 38377, 38378, 38379, 38380 were selected from the ORFs having the nucleotide sequence represented by SEQ ID NO: 2933.

The probes having the nucleotide sequence represented by SEQ ID NOS: 38381, 38382, 38383, 38384, 38385, 38386, 38387, 38388, 38389, 38390, 38391 were selected from the ORFs having the nucleotide sequence represented by SEQ ID NO: 2934.

The probes having the nucleotide sequence represented by SEQ ID NOS: 38392, 38393, 38394, 38395, 38396, 38397, 38398, 38399, 38400, 38401, 38402 were selected from the ORFs having the nucleotide sequence represented by SEQ ID NO: 2935.

The probes having the nucleotide sequence represented by. SEQ ID NOS: 38403, 38404, 38405, 38406, 38407, 38408, 38409, 38410, 38411, 38412, 38413 were selected from the ORFs having the nucleotide sequence represented by SEQ ID NO: 2936.

The probes having the nucleotide sequence represented by SEQ ID NOS: 38414, 38415, 38416, 38417, 38418, 38419, 38420, 38421, 38422, 38423, 38424 were selected from the ORFs having the nucleotide sequence represented by SEQ ID NO: 2937.

The probes having the nucleotide sequence represented by SEQ ID NOS: 38425, 38426, 38427, 38428, 38429, 38430, 38431, 38432, 38433, 38434, 38435 were selected from the ORFs having the nucleotide sequence represented by SEQ ID NO: 2938.

The probes having the nucleotide sequence represented by SEQ ID NOS: 38436, 38437, 38438, 38439, 38440, 38441, 38442, 38443, 38444, 38445, 38446 were selected from the ORFs having the nucleotide sequence represented by SEQ ID NO: 2939.

The probes having the nucleotide sequence represented by SEQ ID NOS: 38447, 38448, 38449, 38450, 38451, 38452, 38453, 38454, 38455, 38456, 38457 were selected from the ORFs having the nucleotide sequence represented by SEQ ID NO: 2940.

The probes having the nucleotide sequence represented by SEQ ID NOS: 38458, 38459, 38460, 38461, 38462, 38463, 38464, 38465, 38466, 38467, 38468 were selected from the ORFs having the nucleotide sequence represented by SEQ ID NO: 2941.

The probes having the nucleotide sequence represented by SEQ ID NOS: 38469, 38470, 38471, 38472, 38473, 38474, 38475, 38476, 38477, 38478, 38479 were selected from the ORFs having the nucleotide sequence represented by SEQ ID NO: 2942.

The probes having the nucleotide sequence represented by SEQ ID NOS: 38480, 38481, 38482, 38483, 38484, 38485, 38486, 38487, 38488, 38489, 38490 were selected from the ORFs having the nucleotide sequence represented by SEQ ID NO: 2943.

The probes having the nucleotide sequence represented by SEQ ID NOS: 38491, 38492, 38493, 38494, 38495, 38496, 38497, 38498, 38499, 38500, 38501 were selected from the ORFs having the nucleotide sequence represented by SEQ ID NO: 2944.

The probes having the nucleotide sequence represented by SEQ ID NOS: 38502, 38503, 38504, 38505, 38506, 38507, 38508, 38509, 38510, 38511, 38512 were selected from the ORFs having the nucleotide sequence represented by SEQ ID NO: 2945.

The probes having the nucleotide sequence represented by SEQ ID NOS: 38513, 38514, 38515, 38516, 38517, 38518, 38519, 38520, 38521, 38522, 38523 were selected from the ORFs having the nucleotide sequence represented by SEQ ID NO: 2946.

The probes having the nucleotide sequence represented by SEQ ID NOS: 38524, 38525, 38526, 38527, 38528, 38529, 38530, 38531, 38532, 38533, 38534 were selected from the ORFs having the nucleotide sequence represented by SEQ ID NO: 2947.

The probes having the nucleotide sequence represented by SEQ ID NOS: 38535, 38536, 38537, 38538, 38539, 38540, 38541, 38542, 38543, 38544, 38545 were selected from the ORFs having the nucleotide sequence represented by SEQ ID NO: 2948.

The probes having the nucleotide sequence represented by SEQ ID NOS: 38546, 38547, 38548, 38549, 38550, 38551, 38552, 38553, 38554, 38555, 38556 were selected from the ORFs having the nucleotide sequence represented by SEQ ID NO: 2949.

The probes having the nucleotide sequence represented by SEQ ID NOS: 38557, 38558, 38559, 38560, 38561, 38562, 38563, 38564, 38565, 38566, 38567 were selected from the ORFs having the nucleotide sequence represented by SEQ ID NO: 2950.

The probes having the nucleotide sequence represented by SEQ ID NOS: 38568, 38569, 38570, 38571, 38572, 38573, 38574, 38575, 38576, 38577, 38578 were selected from the ORFs having the nucleotide sequence represented by SEQ ID NO: 2951.

The probes having the nucleotide sequence represented by SEQ ID NOS: 38579, 38580, 38581, 38582, 38583, 38584, 38585, 38586, 38587, 38588, 38589 were selected from the ORFs having the nucleotide sequence represented by SEQ ID NO: 2952.

The probes having the nucleotide sequence represented by SEQ ID NOS: 38590, 38591, 38592, 38593, 38594, 38595, 38596, 38597, 38598, 38599, 38600 were selected from the ORFs having the nucleotide sequence represented by SEQ ID NO: 2953.

The probes having the nucleotide sequence represented by SEQ ID NOS: 38601, 38602, 38603, 38604, 38605, 38606, 38607, 38608, 38609, 38610, 38611 were selected from the ORFs having the nucleotide sequence represented by SEQ ID NO: 2954.

The probes having the nucleotide sequence represented by SEQ ID NOS: 38612, 38613, 38614, 38615, 38616, 38617, 38618, 38619, 38620, 38621, 38622 were selected from the ORFs having the nucleotide sequence represented by SEQ ID NO: 2955.

The probes having the nucleotide sequence represented by SEQ ID NOS: 38623, 38624, 38625, 38626, 38627, 38628, 38629, 38630, 38631, 38632, 38633 were selected from the ORFs having the nucleotide sequence represented by SEQ ID NO: 2956.

The probes having the nucleotide sequence represented by SEQ ID NOS: 38634, 38635, 38636, 38637, 38638, 38639, 38640, 38641, 38642, 38643, 38644 were selected from the ORFs having the nucleotide sequence represented by SEQ ID NO: 2957.

The probes having the nucleotide sequence represented by SEQ ID NOS: 38645, 38646, 38647, 38648, 38649, 38650, 38651, 38652, 38653, 38654, 38655 were selected from the ORFs having the nucleotide sequence represented by SEQ ID NO: 2958.

The probes having the nucleotide sequence represented by SEQ ID NOS: 38656, 38657, 38658, 38659, 38660, 38661, 38662, 38663, 38664, 38665, 38666 were selected from the ORFs having the nucleotide sequence represented by SEQ ID NO: 2959.

The probes having the nucleotide sequence represented by SEQ ID NOS: 38667, 38668, 38669, 38670, 38671, 38672, 38673, 38674, 38675, 38676, 38677 were selected from the ORFs having the nucleotide sequence represented by SEQ ID NO: 2960.

The probes having the nucleotide sequence represented by SEQ ID NOS: 38678, 38679, 38680, 38681, 38682, 38683, 38684, 38685, 38686, 38687, 38688 were selected from the ORFs having the nucleotide sequence represented by SEQ ID NO: 2961.

The probes having the nucleotide sequence represented by SEQ ID NOS: 38689, 38690, 38691, 38692, 38693, 38694, 38695, 38696, 38697, 38698, 38699 were selected from the ORFs having the nucleotide sequence represented by SEQ ID NO: 2962.

The probes having the nucleotide sequence represented by SEQ ID NOS: 38700, 38701, 38702, 38703, 38704, 38705, 38706, 38707, 38708, 38709, 38710 were selected from the ORFs having the nucleotide sequence represented by SEQ ID NO: 2963.

The probes having the nucleotide sequence represented by SEQ ID NOS: 38711, 38712, 38713, 38714, 38715, 38716, 38717, 38718, 38719, 38720, 38721 were selected from the ORFs having the nucleotide sequence represented by SEQ ID NO: 2964.

The probes having the nucleotide sequence represented by SEQ ID NOS: 38722, 38723, 38724, 38725, 38726, 38727, 38728, 38729, 38730, 38731, 38732 were selected from the ORFs having the nucleotide sequence represented by SEQ ID NO: 2965.

The probes having the nucleotide sequence represented by SEQ ID NOS: 38733, 38734, 38735, 38736, 38737, 38738, 38739, 38740, 38741, 38742, 38743 were selected from the ORFs having the nucleotide sequence represented by SEQ ID NO: 2966.

The probes having the nucleotide sequence represented by SEQ ID NOS: 38744, 38745, 38746, 38747, 38748, 38749, 38750, 38751, 38752, 38753, 38754 were selected from the ORFs having the nucleotide sequence represented by SEQ ID NO: 2967.

The probes having the nucleotide sequence represented by SEQ ID NOS: 38755, 38756, 38757, 38758, 38759, 38760, 38761, 38762, 38763, 38764, 38765 were selected from the ORFs having the nucleotide sequence represented by SEQ ID NO: 2968.

The probes having the nucleotide sequence represented by SEQ ID NOS: 38766, 38767, 38768, 38769, 38770, 38771, 38772, 38773, 38774, 38775, 38776 were selected from the ORFs having the nucleotide sequence represented by SEQ ID NO: 2969.

The probes having the nucleotide sequence represented by SEQ ID NOS: 38777, 38778, 38779, 38780, 38781, 38782, 38783, 38784, 38785, 38786, 38787 were selected from the ORFs having the nucleotide sequence represented by SEQ ID NO: 2970.

The probes having the nucleotide sequence represented by SEQ ID NOS: 38788, 38789, 38790, 38791, 38792, 38793, 38794, 38795, 38796, 38797, 38798 were selected from the ORFs having the nucleotide sequence represented by SEQ ID NO: 2971.

The probes having the nucleotide sequence represented by SEQ ID NOS: 38799, 38800, 38801, 38802, 38803, 38804, 38805, 38806, 38807, 38808, 38809 were selected from the ORFs having the nucleotide sequence represented by SEQ ID NO: 2972.

The probes having the nucleotide sequence represented by SEQ ID NOS: 38810, 38811, 38812, 38813, 38814, 38815, 38816, 38817, 38818, 38819, 38820 were selected from the ORFs having the nucleotide sequence represented by SEQ ID NO: 2973.

The probes having the nucleotide sequence represented by SEQ ID NOS: 38821, 38822, 38823, 38824, 38825, 38826, 38827, 38828, 38829, 38830, 38831 were selected from the ORFs having the nucleotide sequence represented by SEQ ID NO: 2974.

The probes having the nucleotide sequence represented by SEQ ID NOS: 38832, 38833, 38834, 38835, 38836, 38837, 38838, 38839, 38840, 38841, 38842 were selected from the ORFs having the nucleotide sequence represented by SEQ ID NO: 2975.

The probes having the nucleotide sequence represented by SEQ ID NOS: 38843, 38844, 38845, 38846, 38847, 38848, 38849, 38850, 38851, 38852, 38853 were selected from the ORFs having the nucleotide sequence represented by SEQ ID NO: 2976.

The probes having the nucleotide sequence represented by SEQ ID NOS: 38854, 38855, 38856, 38857, 38858, 38859, 38860, 38861, 38862, 38863, 38864 were selected from the ORFs having the nucleotide sequence represented by SEQ ID NO: 2977.

The probes having the nucleotide sequence represented by SEQ ID NOS: 38865, 38866, 38867, 38868, 38869, 38870, 38871, 38872, 38873, 38874, 38875 were selected from the ORFs having the nucleotide sequence represented by SEQ ID NO: 2978.

The probes having the nucleotide sequence represented by SEQ ID NOS: 38876, 38877, 38878, 38879, 38880, 38881, 38882, 38883, 38884, 38885, 38886 were selected from the ORFs having the nucleotide sequence represented by SEQ ID NO: 2979.

The probes having the nucleotide sequence represented by SEQ ID NOS: 38887, 38888, 38889, 38890, 38891, 38892, 38893, 38894, 38895, 38896, 38897 were selected from the ORFs having the nucleotide sequence represented by SEQ ID NO: 2980.

The probes having the nucleotide sequence represented by SEQ ID NOS: 38898, 38899, 38900, 38901, 38902, 38903, 38904, 38905, 38906, 38907, 38908 were selected from the ORFs having the nucleotide sequence represented by SEQ ID NO: 2981.

The probes having the nucleotide sequence represented by SEQ ID NOS: 38909, 38910, 38911, 38912, 38913, 38914, 38915, 38916, 38917, 38918, 38919 were selected from the ORFs having the nucleotide sequence represented by SEQ ID NO: 2982.

The probes having the nucleotide sequence represented by SEQ ID NOS: 38920, 38921, 38922, 38923, 38924, 38925, 38926, 38927, 38928, 38929, 38930 were selected from the ORFs having the nucleotide sequence represented by SEQ ID NO: 2983.

The probes having the nucleotide sequence represented by SEQ ID NOS: 38931, 38932, 38933, 38934, 38935, 38936, 38937, 38938, 38939, 38940, 38941 were selected from the ORFs having the nucleotide sequence represented by SEQ ID NO: 2984.

The probes having the nucleotide sequence represented by SEQ ID NOS: 38942, 38943, 38944, 38945, 38946, 38947, 38948, 38949, 38950, 38951, 38952 were selected from the ORFs having the nucleotide sequence represented by SEQ ID NO: 2985.

The probes having the nucleotide sequence represented by SEQ ID NOS: 38953, 38954, 38955, 38956, 38957, 38958, 38959, 38960, 38961, 38962, 38963 were selected from the ORFs having the nucleotide sequence represented by SEQ ID NO: 2986.

The probes having the nucleotide sequence represented by SEQ ID NOS: 38964, 38965, 38966, 38967, 38968, 38969, 38970, 38971, 38972, 38973, 38974 were selected from the ORFs having the nucleotide sequence represented by SEQ ID NO: 2987.

The probes having the nucleotide sequence represented by SEQ ID NOS: 38975, 38976, 38977, 38978, 38979, 38980, 38981, 38982, 38983, 38984, 38985 were selected from the ORFs having the nucleotide sequence represented by SEQ ID NO: 2988.

The probes having the nucleotide sequence represented by SEQ ID NOS: 38986, 38987, 38988, 38989, 38990, 38991, 38992, 38993, 38994, 38995, 38996 were selected from the ORFs having the nucleotide sequence represented by SEQ ID NO: 2989.

The probes having the nucleotide sequence represented by SEQ ID NOS: 38997, 38998, 38999, 39000, 39001, 39002, 39003, 39004, 39005, 39006, 39007 were selected from the ORFs having the nucleotide sequence represented by SEQ ID NO: 2990.

The probes having the nucleotide sequence represented by SEQ ID NOS: 39008, 39009, 39010, 39011, 39012, 39013, 39014, 39015, 39016, 39017, 39018 were selected from the ORFs having the nucleotide sequence represented by SEQ ID NO: 2991.

The probes having the nucleotide sequence represented by SEQ ID NOS: 39019, 39020, 39021, 39022, 39023, 39024, 39025, 39026, 39027, 39028, 39029 were selected from the ORFs having the nucleotide sequence represented by SEQ ID NO: 2992.

The probes having the nucleotide sequence represented by SEQ ID NOS: 39030, 39031, 39032, 39033, 39034, 39035, 39036, 39037, 39038, 39039, 39040 were selected from the ORFs having the nucleotide sequence represented by SEQ ID NO: 2993.

The probes having the nucleotide sequence represented by SEQ ID NOS: 39041, 39042, 39043, 39044, 39045, 39046, 39047, 39048, 39049, 39050, 39051 were selected from the ORFs having the nucleotide sequence represented by SEQ ID NO: 2994.

The probes having the nucleotide sequence represented by SEQ ID NOS: 39052, 39053, 39054, 39055, 39056, 39057, 39058, 39059, 39060, 39061, 39062 were selected from the ORFs having the nucleotide sequence represented by SEQ ID NO: 2995.

The probes having the nucleotide sequence represented by SEQ ID NOS: 39063, 39064, 39065, 39066, 39067, 39068, 39069, 39070, 39071, 39072, 39073 were selected from the ORFs having the nucleotide sequence represented by SEQ ID NO: 2996.

The probes having the nucleotide sequence represented by SEQ ID NOS: 39074, 39075, 39076, 39077, 39078, 39079, 39080, 39081, 39082, 39083, 39084 were selected from the ORFs having the nucleotide sequence represented by SEQ ID NO: 2997.

The probes having the nucleotide sequence represented by SEQ ID NOS: 39085, 39086, 39087, 39088, 39089, 39090, 39091, 39092, 39093, 39094, 39095 were selected from the ORFs having the nucleotide sequence represented by SEQ ID NO: 2998.

The probes having the nucleotide sequence represented by SEQ ID NOS: 39096, 39097, 39098, 39099, 39100, 39101, 39102, 39103, 39104, 39105, 39106 were selected from the ORFs having the nucleotide sequence represented by SEQ ID NO: 2999.

The probes having the nucleotide sequence represented by SEQ ID NOS: 39107, 39108, 39109, 39110, 39111, 39112, 39113, 39114, 39115, 39116, 39117 were selected from the ORFs having the nucleotide sequence represented by SEQ ID NO: 3000.

The probes having the nucleotide sequence represented by SEQ ID NOS: 39118, 39119, 39120, 39121, 39122, 39123, 39124, 39125, 39126, 39127, 39128 were selected from the ORFs having the nucleotide sequence represented by SEQ ID NO: 3001.

The probes having the nucleotide sequence represented by SEQ ID NOS: 39129, 39130, 39131, 39132, 39133, 39134, 39135, 39136, 39137, 39138, 39139 were selected from the ORFs having the nucleotide sequence represented by SEQ ID NO: 3002.

The probes having the nucleotide sequence represented by SEQ ID NOS: 39140, 39141, 39142, 39143, 39144, 39145, 39146, 39147, 39148, 39149, 39150 were selected from the ORFs having the nucleotide sequence represented by SEQ ID NO: 3003.

The probes having the nucleotide sequence represented by SEQ ID NOS: 39151, 39152, 39153, 39154, 39155, 39156, 39157, 39158, 39159, 39160, 39161 were selected from the ORFs having the nucleotide sequence represented by SEQ ID NO: 3004.

The probes having the nucleotide sequence represented by SEQ ID NOS: 39162, 39163, 39164, 39165, 39166, 39167, 39168, 39169, 39170, 39171, 39172 were selected from the ORFs having the nucleotide sequence represented by SEQ ID NO: 3005.

The probes having the nucleotide sequence represented by SEQ ID NOS: 39173, 39174, 39175, 39176, 39177, 39178, 39179, 39180, 39181, 39182, 39183 were selected from the ORFs having the nucleotide sequence represented by SEQ ID NO: 3006.

The probes having the nucleotide sequence represented by SEQ ID NOS: 39184, 39185, 39186, 39187, 39188, 39189, 39190, 39191, 39192, 39193, 39194 were selected from the ORFs having the nucleotide sequence represented by SEQ ID NO: 3007.

The probes having the nucleotide sequence represented by SEQ ID NOS: 39195, 39196, 39197, 39198, 39199, 39200, 39201, 39202, 39203, 39204, 39205 were selected from the ORFs having the nucleotide sequence represented by SEQ ID NO: 3008.

The probes having the nucleotide sequence represented by SEQ ID NOS: 39206, 39207, 39208, 39209, 39210, 39211, 39212, 39213, 39214, 39215, 39216 were selected from the ORFs having the nucleotide sequence represented by SEQ ID NO: 3009.

The probes having the nucleotide sequence represented by SEQ ID NOS: 39217, 39218, 39219, 39220, 39221, 39222, 39223, 39224, 39225, 39226, 39227 were selected from the ORFs having the nucleotide sequence represented by SEQ ID NO: 3010.

The probes having the nucleotide sequence represented by SEQ ID NOS: 39228, 39229, 39230, 39231, 39232, 39233, 39234, 39235, 39236, 39237, 39238 were selected from the ORFs having the nucleotide sequence represented by SEQ ID NO: 3011.

The probes having the nucleotide sequence represented by SEQ ID NOS: 39239, 39240, 39241, 39242, 39243, 39244, 39245, 39246, 39247, 39248, 39249 were selected from the ORFs having the nucleotide sequence represented by SEQ ID NO: 3012.

The probes having the nucleotide sequence represented by SEQ ID NOS: 39250, 39251, 39252, 39253, 39254, 39255, 39256, 39257, 39258, 39259, 39260 were selected from the ORFs having the nucleotide sequence represented by SEQ ID NO: 3013.

The probes having the nucleotide sequence represented by SEQ ID NOS: 39261, 39262, 39263, 39264, 39265, 39266, 39267, 39268, 39269, 39270, 39271 were selected from the ORFs having the nucleotide sequence represented by SEQ ID NO: 3014.

The probes having the nucleotide sequence represented by SEQ ID NOS: 39272, 39273, 39274, 39275, 39276, 39277, 39278, 39279, 39280, 39281, 39282 were selected from the ORFs having the nucleotide sequence represented by SEQ ID NO: 3015.

The probes having the nucleotide sequence represented by SEQ ID NOS: 39283, 39284, 39285, 39286, 39287, 39288, 39289, 39290, 39291, 39292, 39293 were selected from the ORFs having the nucleotide sequence represented by SEQ ID NO: 3016.

The probes having the nucleotide sequence represented by SEQ ID NOS: 39294, 39295, 39296, 39297, 39298, 39299, 39300, 39301, 39302, 39303, 39304 were selected from the ORFs having the nucleotide sequence represented by SEQ ID NO: 3017.

The probes having the nucleotide sequence represented by SEQ ID NOS: 39305, 39306, 39307, 39308, 39309, 39310, 39311, 39312, 39313, 39314, 39315 were selected from the ORFs having the nucleotide sequence represented by SEQ ID NO: 3018.

The probes having the nucleotide sequence represented by SEQ ID NOS: 39316, 39317, 39318, 39319, 39320, 39321, 39322, 39323, 39324, 39325, 39326 were selected from the ORFs having the nucleotide sequence represented by SEQ ID NO: 3019.

The probes having the nucleotide sequence represented by SEQ ID NOS: 39327, 39328, 39329, 39330, 39331, 39332, 39333, 39334, 39335, 39336, 39337 were selected from the ORFs having the nucleotide sequence represented by SEQ ID NO: 3020.

The probes having the nucleotide sequence represented by SEQ ID NOS: 39338, 39339, 39340, 39341, 39342, 39343, 39344, 39345, 39346, 39347, 39348 were selected from the ORFs having the nucleotide sequence represented by SEQ ID NO: 3021.

The probes having the nucleotide sequence represented by SEQ ID NOS: 39349, 39350, 39351, 39352, 39353, 39354, 39355, 39356, 39357, 39358, 39359 were selected from the ORFs having the nucleotide sequence represented by SEQ ID NO: 3022.

The probes having the nucleotide sequence represented by SEQ ID NOS: 39360, 39361, 39362, 39363, 39364, 39365, 39366, 39367, 39368, 39369, 39370 were selected from the ORFs having the nucleotide sequence represented by SEQ ID NO: 3023.

The probes having the nucleotide sequence represented by SEQ ID NOS: 39371, 39372, 39373, 39374, 39375, 39376, 39377, 39378, 39379, 39380, 39381 were selected from the ORFs having the nucleotide sequence represented by SEQ ID NO: 3024.

The probes having the nucleotide sequence represented by SEQ ID NOS: 39382, 39383, 39384, 39385, 39386, 39387, 39388, 39389, 39390, 39391, 39392 were selected from the ORFs having the nucleotide sequence represented by SEQ ID NO: 3025.

The probes having the nucleotide sequence represented by SEQ ID NOS: 39393, 39394, 39395, 39396, 39397, 39398, 39399, 39400, 39401, 39402, 39403 were selected from the ORFs having the nucleotide sequence represented by SEQ ID NO: 3026.

The probes having the nucleotide sequence represented by SEQ ID NOS: 39404, 39405, 39406, 39407, 39408, 39409, 39410, 39411, 39412, 39413, 39414 were selected from the ORFs having the nucleotide sequence represented by SEQ ID NO: 3027.

The probes having the nucleotide sequence represented by SEQ ID NOS: 39415, 39416, 39417, 39418, 39419, 39420, 39421, 39422, 39423, 39424, 39425 were selected from the ORFs having the nucleotide sequence represented by SEQ ID NO: 3028.

The probes having the nucleotide sequence represented by SEQ ID NOS: 39426, 39427, 39428, 39429, 39430, 39431, 39432, 39433, 39434, 39435, 39436 were selected from the ORFs having the nucleotide sequence represented by SEQ ID NO: 3029.

The probes having the nucleotide sequence represented by SEQ ID NOS: 39437, 39438, 39439, 39440, 39441, 39442, 39443, 39444, 39445, 39446, 39447 were selected from the ORFs having the nucleotide sequence represented by SEQ ID NO: 3030.

The probes having the nucleotide sequence represented by SEQ ID NOS: 39448, 39449, 39450, 39451, 39452, 39453, 39454, 39455, 39456, 39457, 39458 were selected from the ORFs having the nucleotide sequence represented by SEQ ID NO: 3031.

The probes having the nucleotide sequence represented by SEQ ID NOS: 39459, 39460, 39461, 39462, 39463, 39464, 39465, 39466, 39467, 39468, 39469 were selected from the ORFs having the nucleotide sequence represented by SEQ ID NO: 3032.

The probes having the nucleotide sequence represented by SEQ ID NOS: 39470, 39471, 39472, 39473, 39474, 39475, 39476, 39477, 39478, 39479, 39480 were selected from the ORFs having the nucleotide sequence represented by SEQ ID NO: 3033.

The probes having the nucleotide sequence represented by SEQ ID NOS: 39481, 39482, 39483, 39484, 39485, 39486, 39487, 39488, 39489, 39490, 39491 were selected from the ORFs having the nucleotide sequence represented by SEQ ID NO: 3034.

The probes having the nucleotide sequence represented by SEQ ID NOS: 39492, 39493, 39494, 39495, 39496, 39497, 39498, 39499, 39500, 39501, 39502 were selected from the ORFs having the nucleotide sequence represented by SEQ ID NO: 3035.

The probes having the nucleotide sequence represented by SEQ ID NOS: 39503, 39504, 39505, 39506, 39507, 39508, 39509,X39510, 39511, 39512, 39513 were selected from the ORFs having the nucleotide sequence represented by SEQ ID NO: 3036.

The probes having the nucleotide sequence represented by SEQ ID NOS: 39514, 39515, 39516, 39517, 39518, 39519, 39520, 39521, 39522, 39523, 39524 were selected from the ORFs having the nucleotide sequence represented by SEQ ID NO: 3037.

The probes having the nucleotide sequence represented by SEQ ID NOS: 39525, 39526, 39527, 39528, 39529, 39530, 39531, 39532, 39533, 39534, 39535 were selected from the ORFs having the nucleotide sequence represented by SEQ ID NO: 3038.

The probes having the nucleotide sequence represented by SEQ ID NOS: 39536, 39537, 39538, 39539, 39540, 39541, 39542, 39543, 39544, 39545, 39546 were selected from the ORFs having the nucleotide sequence represented by SEQ ID NO: 3039.

The probes having the nucleotide sequence represented by SEQ ID NOS: 39547, 39548, 39549, 39550, 39551, 39552, 39553, 39554, 39555, 39556, 39557 were selected from the ORFs having the nucleotide sequence represented by SEQ ID NO: 3040.

The probes having the nucleotide sequence represented by SEQ ID NOS: 39558, 39559, 39560, 39561, 39562, 39563, 39564, 39565, 39566, 39567, 39568 were selected from the ORFs having the nucleotide sequence represented by SEQ ID NO: 3041.

The probes having the nucleotide sequence represented by SEQ ID NOS: 39569, 39570, 39571, 39572, 39573, 39574, 39575, 39576, 39577, 39578, 39579 were selected from the ORFs having the nucleotide sequence represented by SEQ ID NO: 3042.

The probes having the nucleotide sequence represented by SEQ ID NOS: 39580, 39581, 39582, 39583, 39584, 39585, 39586, 39587, 39588, 39589, 39590 were selected from the ORFs having the nucleotide sequence represented by SEQ ID NO: 3043.

The probes having the nucleotide sequence represented by SEQ ID NOS: 39591, 39592, 39593, 39594, 39595, 39596, 39597, 39598, 39599, 39600, 39601 were selected from the ORFs having the nucleotide sequence represented by SEQ ID NO: 3044.

The probes having the nucleotide sequence represented by SEQ ID NOS: 39602, 39603, 39604, 39605, 39606, 39607, 39608, 39609, 39610, 39611, 39612 were selected from the ORFs having the nucleotide sequence represented by SEQ ID NO: 3045.

The probes having the nucleotide sequence represented by SEQ ID NOS: 39613, 39614, 39615, 39616, 39617, 39618, 39619, 39620, 39621, 39622, 39623 were selected from the ORFs having the nucleotide sequence represented by SEQ ID NO: 3046.

The probes having the nucleotide sequence represented by SEQ ID NOS: 39624, 39625, 39626, 39627, 39628, 39629, 39630, 39631, 39632, 39633, 39634 were selected from the ORFs having the nucleotide sequence represented by SEQ ID NO: 3047.

The probes having the nucleotide sequence represented by SEQ ID NOS: 39635, 39636, 39637, 39638, 39639, 39640, 39641, 39642, 39643, 39644, 39645 were selected from the ORFs having the nucleotide sequence represented by SEQ ID NO: 3048.

The probes having the nucleotide sequence represented by SEQ ID NOS: 39646, 39647, 39648, 39649, 39650, 39651, 39652, 39653, 39654, 39655, 39656 were selected from the ORFs having the nucleotide sequence represented by SEQ ID NO: 3049.

The probes having the nucleotide sequence represented by SEQ ID NOS: 39657, 39658, 39659, 39660, 39661, 39662, 39663, 39664, 39665, 39666, 39667 were selected from the ORFs having the nucleotide sequence represented by SEQ ID NO: 3050.

The probes having the nucleotide sequence represented by SEQ ID NOS: 39668, 39669, 39670, 39671, 39672, 39673, 39674, 39675, 39676, 39677, 39678 were selected from the ORFs having the nucleotide sequence represented by SEQ ID NO: 3051.

The probes having the nucleotide sequence represented by SEQ ID NOS: 39679, 39680, 39681, 39682, 39683, 39684, 39685, 39686, 39687, 39688, 39689 were selected from the ORFs having the nucleotide sequence represented by SEQ ID NO: 3052.

The probes having the nucleotide sequence represented by SEQ ID NOS: 39690, 39691, 39692, 39693, 39694, 39695, 39696, 39697, 39698, 39699, 39700 were selected from the ORFs having the nucleotide sequence represented by SEQ ID NO: 3053.

The probes having the nucleotide sequence represented by SEQ ID NOS: 39701, 39702, 39703, 39704, 39705, 39706, 39707, 39708, 39709, 39710, 39711 were selected from the ORFs having the nucleotide sequence represented by SEQ ID NO: 3054.

The probes having the nucleotide sequence represented by SEQ ID NOS: 39712, 39713, 39714, 39715, 39716, 39717, 39718, 39719, 39720, 39721, 39722 were selected from the ORFs having the nucleotide sequence represented by SEQ ID NO: 3055.

The probes having the nucleotide sequence represented by SEQ ID NOS: 39723, 39724, 39725, 39726, 39727, 39728, 39729, 39730, 39731, 39732, 39733 were selected from the ORFs having the nucleotide sequence represented by SEQ ID NO: 3056.

The probes having the nucleotide sequence represented by SEQ ID NOS: 39734, 39735, 39736, 39737, 39738, 39739, 39740, 39741, 39742, 39743, 39744 were selected from the ORFs having the nucleotide sequence represented by SEQ ID NO: 3057.

The probes having the nucleotide sequence represented by SEQ ID NOS: 39745, 39746, 39747, 39748, 39749, 39750, 39751, 39752, 39753, 39754, 39755 were selected from the ORFs having the nucleotide sequence represented by SEQ ID NO: 3058.

The probes having the nucleotide sequence represented by SEQ ID NOS: 39756, 39757, 39758, 39759, 39760, 39761, 39762, 39763, 39764, 39765, 39766 were selected from the ORFs having the nucleotide sequence represented by SEQ ID NO: 3059.

The probes having the nucleotide sequence represented by SEQ ID NOS: 39767, 39768, 39769, 39770, 39771, 39772, 39773, 39774, 39775, 39776, 39777 were selected from the ORFs having the nucleotide sequence represented by SEQ ID NO: 3060.

The probes having the nucleotide sequence represented by SEQ ID NOS: 39778, 39779, 39780, 39781, 39782, 39783, 39784, 39785, 39786, 39787, 39788 were selected from the ORFs having the nucleotide sequence represented by SEQ ID NO: 3061.

The probes having the nucleotide sequence represented by SEQ ID NOS: 39789, 39790, 39791, 39792, 39793, 39794, 39795, 39796, 39797, 39798, 39799 were selected from the ORFs having the nucleotide sequence represented by SEQ ID NO: 3062.

The probes having the nucleotide sequence represented by SEQ ID NOS: 39800, 39801, 39802, 39803, 39804, 39805, 39806, 39807, 39808, 39809, 39810 were selected from the ORFs having the nucleotide sequence represented by SEQ ID NO: 3063.

The probes having the nucleotide sequence represented by SEQ ID NOS: 39811, 39812, 39813, 39814, 39815, 39816, 39817, 39818, 39819, 39820, 39821 were selected from the ORFs having the nucleotide sequence represented by SEQ ID NO: 3064.

The probes having the nucleotide sequence represented by SEQ ID NOS: 39822, 39823, 39824, 39825, 39826, 39827, 39828, 39829, 39830, 39831, 39832 were selected from the ORFs having the nucleotide sequence represented by SEQ ID NO: 3065.

The probes having the nucleotide sequence represented by SEQ ID NOS: 39833, 39834, 39835, 39836, 39837, 39838, 39839, 39840, 39841, 39842, 39843 were selected from the ORFs having the nucleotide sequence represented by SEQ ID NO: 3066.

The probes having the nucleotide sequence represented by SEQ ID NOS: 39844, 39845, 39846, 39847, 39848, 39849, 39850, 39851, 39852, 39853, 39854 were selected from the ORFs having the nucleotide sequence represented by SEQ ID NO: 3067.

The probes having the nucleotide sequence represented by SEQ ID NOS: 39855, 39856, 39857, 39858, 39859, 39860, 39861, 39862, 39863, 39864, 39865 were selected from the ORFs having the nucleotide sequence represented by SEQ ID NO: 3068.

The probes having the nucleotide sequence represented by SEQ ID NOS: 39866, 39867, 39868, 39869, 39870, 39871, 39872, 39873, 39874, 39875, 39876 were selected from the ORFs having the nucleotide sequence represented by SEQ ID NO: 3069.

The probes having the nucleotide sequence represented by SEQ ID NOS: 39877, 39878, 39879, 39880, 39881, 39882, 39883, 39884, 39885, 39886, 39887 were selected from the ORFs having the nucleotide sequence represented by SEQ ID NO: 3070.

The probes having the nucleotide sequence represented by SEQ ID NOS: 39888, 39889, 39890, 39891, 39892, 39893, 39894, 39895, 39896, 39897, 39898 were selected from the ORFs having the nucleotide sequence represented by SEQ ID NO: 3071.

The probes having the nucleotide sequence represented by SEQ ID NOS: 39899, 39900, 39901, 39902, 39903, 39904, 39905, 39906, 39907, 39908, 39909 were selected from the ORFs having the nucleotide sequence represented by SEQ ID NO: 3072.

The probes having the nucleotide sequence represented by SEQ ID NOS: 39910, 39911, 39912, 39913, 39914, 39915, 39916, 39917, 39918, 39919, 39920 were selected from the ORFs having the nucleotide sequence represented by SEQ ID NO: 3073.

The probes having the nucleotide sequence represented by SEQ ID NOS: 39921, 39922, 39923, 39924, 39925, 39926, 39927, 39928, 39929, 39930, 39931 were selected from the ORFs having the nucleotide sequence represented by SEQ ID NO: 3074.

The probes having the nucleotide sequence represented by SEQ ID NOS: 39932, 39933, 39934, 39935, 39936, 39937, 39938, 39939, 39940, 39941, 39942 were selected from the ORFs having the nucleotide sequence represented by SEQ ID NO: 3075.

The probes having the nucleotide sequence represented by SEQ ID NOS: 39943, 39944, 39945, 39946, 39947, 39948, 39949, 39950, 39951, 39952, 39953 were selected from the ORFs having the nucleotide sequence represented by SEQ ID NO: 3076.

The probes having the nucleotide sequence represented by SEQ ID NOS: 39954, 39955, 39956, 39957, 39958, 39959, 39960, 39961, 39962, 39963, 39964 were selected from the ORFs having the nucleotide sequence represented by SEQ ID NO: 3077.

The probes having the nucleotide sequence represented by SEQ ID NOS: 39965, 39966, 39967, 39968, 39969, 39970, 39971, 39972, 39973, 39974, 39975 were selected from the ORFs having the nucleotide sequence represented by SEQ ID NO: 3078.

The probes having the nucleotide sequence represented by SEQ ID NOS: 39976, 39977, 39978, 39979, 39980, 39981, 39982, 39983, 39984, 39985, 39986 were selected from the ORFs having the nucleotide sequence represented by SEQ ID NO: 3079.

The probes having the nucleotide sequence represented by SEQ ID NOS: 39987, 39988, 39989, 39990, 39991, 39992, 39993, 39994, 39995, 39996, 39997 were selected from the ORFs having the nucleotide sequence represented by SEQ ID NO: 3080.

The probes having the nucleotide sequence represented by SEQ ID NOS: 39998, 39999, 40000, 40001, 40002, 40003, 40004, 40005, 40006, 40007, 40008 were selected from the ORFs having the nucleotide sequence represented by SEQ ID NO: 3081.

The probes having the nucleotide sequence represented by SEQ ID NOS: 40009, 40010, 40011, 40012, 40013, 40014, 40015, 40016, 40017, 40018, 40019 were selected from the ORFs having the nucleotide sequence represented by SEQ ID NO: 3082.

The probes having the nucleotide sequence represented by SEQ ID NOS: 40020, 40021, 40022, 40023, 40024, 40025, 40026, 40027, 40028, 40029, 40030 were selected from the ORFs having the nucleotide sequence represented by SEQ ID NO: 3083.

The probes having the nucleotide sequence represented by SEQ ID NOS: 40031, 40032, 40033, 40034, 40035, 40036, 40037, 40038, 40039, 40040, 40041 were selected from the ORFs having the nucleotide sequence represented by SEQ ID NO: 3084.

The probes having the nucleotide sequence represented by SEQ ID NOS: 40042, 40043, 40044, 40045, 40046, 40047, 40048, 40049, 40050, 40051, 40052 were selected from the ORFs having the nucleotide sequence represented by SEQ ID NO: 3085.

The probes having the nucleotide sequence represented by SEQ ID NOS: 40053, 40054, 40055, 40056, 40057, 40058, 40059, 40060, 40061, 40062, 40063 were selected from the ORFs having the nucleotide sequence represented by SEQ ID NO: 3086.

The probes having the nucleotide sequence represented by SEQ ID NOS: 40064, 40065, 40066, 40067, 40068, 40069, 40070, 40071, 40072, 40073, 40074 were selected from the ORFs having the nucleotide sequence represented by SEQ ID NO: 3087.

The probes having the nucleotide sequence represented by SEQ ID NOS: 40075, 40076, 40077, 40078, 40079, 40080, 40081, 40082, 40083, 40084, 40085 were selected from the ORFs having the nucleotide sequence represented by SEQ ID NO: 3088.

The probes having the nucleotide sequence represented by SEQ ID NOS: 40086, 40087, 40088, 40089, 40090, 40091, 40092, 40093, 40094, 40095, 40096 were selected from the ORFs having the nucleotide sequence represented by SEQ ID NO: 3089.

The probes having the nucleotide sequence represented by SEQ ID NOS: 40097, 40098, 40099, 40100, 40101, 40102, 40103, 40104, 40105, 40106, 40107 were selected from the ORFs having the nucleotide sequence represented by SEQ ID NO: 3090.

The probes having the nucleotide sequence represented by SEQ ID NOS: 40108, 40109, 40110, 40111, 40112, 40113, 40114, 40115, 40116, 40117, 40118 were selected from the ORFs having the nucleotide sequence represented by SEQ ID NO: 3091.

The probes having the nucleotide sequence represented by SEQ ID NOS: 40119, 40120, 40121, 40122, 40123, 40124, 40125, 40126, 40127, 40128, 40129 were selected from the ORFs having the nucleotide sequence represented by SEQ ID NO: 3092.

The probes having the nucleotide sequence represented by SEQ ID NOS: 40130, 40131, 40132, 40133, 40134, 40135, 40136, 40137, 40138, 40139, 40140 were selected from the ORFs having the nucleotide sequence represented by SEQ ID NO: 3093.

The probes having the nucleotide sequence represented by SEQ ID NOS: 40141, 40142, 40143, 40144, 40145, 40146, 40147, 40148, 40149, 40150, 40151 were selected from the ORFs having the nucleotide sequence represented by SEQ ID NO: 3094.

The probes having the nucleotide sequence represented by SEQ ID NOS: 40152, 40153, 40154, 40155, 40156, 40157, 40158, 40159, 40160, 40161, 40162 were selected from the ORFs having the nucleotide sequence represented by SEQ ID NO: 3095.

The probes having the nucleotide sequence represented by SEQ ID NOS: 40163, 40164, 40165, 40166, 40167, 40168, 40169, 40170, 40171, 40172, 40173 were selected from the ORFs having the nucleotide sequence represented by SEQ ID NO: 3096.

The probes having the nucleotide sequence represented by SEQ ID NOS: 40174, 40175, 40176, 40177, 40178, 40179, 40180, 40181, 40182, 40183, 40184 were selected from the ORFs having the nucleotide sequence represented by SEQ ID NO: 3097.

The probes having the nucleotide sequence represented by SEQ ID NOS: 40185, 40186, 40187, 40188, 40189, 40190, 40191, 40192, 40193, 40194, 40195 were selected from the ORFs having the nucleotide sequence represented by SEQ ID NO: 3098.

The probes having the nucleotide sequence represented by SEQ ID NOS: 40196, 40197, 40198, 40199, 40200, 40201, 40202, 40203, 40204, 40205, 40206 were selected from the ORFs having the nucleotide sequence represented by SEQ ID NO: 3099.

The probes having the nucleotide sequence represented by SEQ ID NOS: 40207, 40208, 40209, 40210, 40211, 40212, 40213, 40214, 40215, 40216, 40217 were selected from the ORFs having the nucleotide sequence represented by SEQ ID NO: 3100.

The probes having the nucleotide sequence represented by SEQ ID NOS: 40218, 40219, 40220, 40221, 40222, 40223, 40224, 40225, 40226, 40227, 40228 were selected from the ORFs having the nucleotide sequence represented by SEQ ID NO: 3101.

The probes having the nucleotide sequence represented by SEQ ID NOS: 40229, 40230, 40231, 40232, 40233, 40234, 40235, 40236, 40237, 40238, 40239 were selected from the ORFs having the nucleotide sequence represented by SEQ ID NO: 3102.

The probes having the nucleotide sequence represented by SEQ ID NOS: 40240, 40241, 40242, 40243, 40244, 40245, 40246, 40247, 40248, 40249, 40250 were selected from the ORFs having the nucleotide sequence represented by SEQ ID NO: 3103.

The probes having the nucleotide sequence represented by SEQ ID NOS: 40251, 40252, 40253, 40254, 40255, 40256, 40257, 40258, 40259, 40260, 40261 were selected from the ORFs having the nucleotide sequence represented by SEQ ID NO: 3104.

The probes having the nucleotide sequence represented by SEQ ID NOS: 40262, 40263, 40264, 40265, 40266, 40267, 40268, 40269, 40270, 40271, 40272 were selected from the ORFs having the nucleotide sequence represented by SEQ ID NO: 3105.

The probes having the nucleotide sequence represented by SEQ ID NOS: 40273, 40274, 40275, 40276, 40277, 40278, 40279, 40280, 40281, 40282, 40283 were selected from the ORFs having the nucleotide sequence represented by SEQ ID NO: 3106.

The probes having the nucleotide sequence represented by SEQ ID NOS: 40284, 40285, 40286, 40287, 40288, 40289, 40290, 40291, 40292, 40293, 40294 were selected from the ORFs having the nucleotide sequence represented by SEQ ID NO: 3107.

The probes having the nucleotide sequence represented by SEQ ID NOS: 40295, 40296, 40297, 40298, 40299, 40300, 40301, 40302, 40303, 40304, 40305 were selected from the ORFs having the nucleotide sequence represented by SEQ ID NO: 3108.

The probes having the nucleotide sequence represented by SEQ ID NOS: 40306, 40307, 40308, 40309, 40310, 40311, 40312, 40313, 40314, 40315, 40316 were selected from the ORFs having the nucleotide sequence represented by SEQ ID NO: 3109.

The probes having the nucleotide sequence represented by SEQ ID NOS: 40317, 40318, 40319, 40320, 40321, 40322, 40323, 40324, 40325, 40326, 40327 were selected from the ORFs having the nucleotide sequence represented by SEQ ID NO: 3110.

The probes having the nucleotide sequence represented by SEQ ID NOS: 40328, 40329, 40330, 40331, 40332, 40333, 40334, 40335, 40336, 40337, 40338 were selected from the ORFs having the nucleotide sequence represented by SEQ ID NO: 3111.

The probes having the nucleotide sequence represented by SEQ ID NOS: 40339, 40340, 40341, 40342, 40343, 40344, 40345, 40346, 40347, 40348, 40349 were selected from the ORFs having the nucleotide sequence represented by SEQ ID NO: 3112.

The probes having the nucleotide sequence represented by SEQ ID NOS: 40350, 40351, 40352, 40353, 40354, 40355, 40356, 40357, 40358, 40359, 40360 were selected from the ORFs having the nucleotide sequence represented by SEQ ID NO: 3113.

The probes having the nucleotide sequence represented by SEQ ID NOS: 40361, 40362, 40363, 40364, 40365, 40366, 40367, 40368, 40369, 40370, 40371 were selected from the ORFs having the nucleotide sequence represented by SEQ ID NO: 3114.

The probes having the nucleotide sequence represented by SEQ ID NOS: 40372, 40373, 40374, 40375, 40376, 40377, 40378, 40379, 40380, 40381, 40382 were selected from the ORFs having the nucleotide sequence represented by SEQ ID NO: 3115.

The probes having the nucleotide sequence represented by SEQ ID NOS: 40383, 40384, 40385, 40386, 40387, 40388, 40389, 40390, 40391, 40392, 40393 were selected from the ORFs having the nucleotide sequence represented by SEQ ID NO: 3116.

The probes having the nucleotide sequence represented by SEQ ID NOS: 40394, 40395, 40396, 40397, 40398, 40399, 40400, 40401, 40402, 40403, 40404 were selected from the ORFs having the nucleotide sequence represented by SEQ ID NO: 3117.

The probes having the nucleotide sequence represented by SEQ ID NOS: 40405, 40406, 40407, 40408, 40409, 40410, 40411, 40412, 40413, 40414, 40415 were selected from the ORFs having the nucleotide sequence represented by SEQ ID NO: 3118.

The probes having the nucleotide sequence represented by SEQ ID NOS: 40416, 40417, 40418, 40419, 40420, 40421, 40422, 40423, 40424, 40425, 40426 were selected from the ORFs having the nucleotide sequence represented by SEQ ID NO: 3119.

The probes having the nucleotide sequence represented by SEQ ID NOS: 40427, 40428, 40429, 40430, 40431, 40432, 40433, 40434, 40435, 40436, 40437 were selected from the ORFs having the nucleotide sequence represented by SEQ ID NO: 3120.

The probes having the nucleotide sequence represented by SEQ ID NOS: 40438, 40439, 40440, 40441, 40442, 40443, 40444, 40445, 40446, 40447, 40448 were selected from the ORFs having the nucleotide sequence represented by SEQ ID NO: 3121.

The probes having the nucleotide sequence represented by SEQ ID NOS: 40449, 40450, 40451, 40452, 40453, 40454, 40455, 40456, 40457, 40458, 40459 were selected from the ORFs having the nucleotide sequence represented by SEQ ID NO: 3122.

The probes having the nucleotide sequence represented by SEQ ID NOS: 40460, 40461, 40462, 40463, 40464, 40465, 40466, 40467, 40468, 40469, 40470 were selected from the ORFs having the nucleotide sequence represented by SEQ ID NO: 3123.

The probes having the nucleotide sequence represented by SEQ ID NOS: 40471, 40472, 40473, 40474, 40475, 40476, 40477, 40478, 40479, 40480, 40481 were selected from the ORFs having the nucleotide sequence represented by SEQ ID NO: 3124.

The probes having the nucleotide sequence represented by SEQ ID NOS: 40482, 40483, 40484, 40485, 40486, 40487, 40488, 40489, 40490, 40491, 40492 were selected from the ORFs having the nucleotide sequence represented by SEQ ID NO: 3125.

The probes having the nucleotide sequence represented by SEQ ID NOS: 40493, 40494, 40495, 40496, 40497, 40498, 40499, 40500, 40501, 40502, 40503 were selected from the ORFs having the nucleotide sequence represented by SEQ ID NO: 3126.

The probes having the nucleotide sequence represented by SEQ ID NOS: 40504, 40505, 40506, 40507, 40508, 40509, 40510, 40511, 40512, 40513, 40514 were selected from the ORFs having the nucleotide sequence represented by SEQ ID NO: 3127.

The probes having the nucleotide sequence represented by SEQ ID NOS: 40515, 40516, 40517, 40518, 40519, 40520, 40521, 40522, 40523, 40524, 40525 were selected from the ORFs having the nucleotide sequence represented by SEQ ID NO: 3128.

The probes having the nucleotide sequence represented by SEQ ID NOS: 40526, 40527, 40528, 40529, 40530, 40531, 40532, 40533, 40534, 40535, 40536 were selected from the ORFs having the nucleotide sequence represented by SEQ ID NO: 3129.

The probes having the nucleotide sequence represented by SEQ ID NOS: 40537, 40538, 40539, 40540, 40541, 40542, 40543, 40544, 40545, 40546, 40547 were selected from the ORFs having the nucleotide sequence represented by SEQ ID NO: 3130.

The probes having the nucleotide sequence represented by SEQ ID NOS: 40548, 40549, 40550, 40551, 40552, 40553, 40554, 40555, 40556, 40557, 40558 were selected from the ORFs having the nucleotide sequence represented by SEQ ID NO: 3131.

The probes having the nucleotide sequence represented by SEQ ID NOS: 40559, 40560, 40561, 40562, 40563, 40564, 40565, 40566, 40567, 40568, 40569 were selected from the ORFs having the nucleotide sequence represented by SEQ ID NO: 3132.

The probes having the nucleotide sequence represented by SEQ ID NOS: 40570, 40571, 40572, 40573, 40574, 40575, 40576, 40577, 40578, 40579, 40580 were selected from the ORFs having the nucleotide sequence represented by SEQ ID NO: 3133.

The probes having the nucleotide sequence represented by SEQ ID NOS: 40581, 40582, 40583, 40584, 40585, 40586, 40587, 40588, 40589, 40590, 40591 were selected from the ORFs having the nucleotide sequence represented by SEQ ID NO: 3134.

The probes having the nucleotide sequence represented by SEQ ID NOS: 40592, 40593, 40594, 40595, 40596, 40597, 40598, 40599, 40600, 40601, 40602 were selected from the ORFs having the nucleotide sequence represented by SEQ ID NO: 3135.

The probes having the nucleotide sequence represented by SEQ ID NOS: 40603, 40604, 40605, 40606, 40607, 40608, 40609, 40610, 40611, 40612, 40613 were selected from the ORFs having the nucleotide sequence represented by SEQ ID NO: 3136.

The probes having the nucleotide sequence represented by SEQ ID NOS: 40614, 40615, 40616, 40617, 40618, 40619, 40620, 40621, 40622, 40623, 40624 were selected from the ORFs having the nucleotide sequence represented by SEQ ID NO: 3137.

The probes having the nucleotide sequence represented by SEQ ID NOS: 40625, 40626, 40627, 40628, 40629, 40630, 40631, 40632, 40633, 40634, 40635 were selected from the ORFs having the nucleotide sequence represented by SEQ ID NO: 3138.

The probes having the nucleotide sequence represented by SEQ ID NOS: 40636, 40637, 40638, 40639, 40640, 40641, 40642, 40643, 40644, 40645, 40646 were selected from the ORFs having the nucleotide sequence represented by SEQ ID NO: 3139.

The probes having the nucleotide sequence represented by SEQ ID NOS: 40647, 40648, 40649, 40650, 40651, 40652, 40653, 40654, 40655, 40656, 40657 were selected from the ORFs having the nucleotide sequence represented by SEQ ID NO: 3140.

The probes having the nucleotide sequence represented by SEQ ID NOS: 40658, 40659, 40660, 40661, 40662, 40663, 40664, 40665, 40666, 40667, 40668 were selected from the ORFs having the nucleotide sequence represented by SEQ ID NO: 3141.

The probes having the nucleotide sequence represented by SEQ ID NOS: 40669, 40670, 40671, 40672, 40673, 40674, 40675, 40676, 40677, 40678, 40679 were selected from the ORFs having the nucleotide sequence represented by SEQ ID NO: 3142.

The probes having the nucleotide sequence represented by SEQ ID NOS: 40680, 40681, 40682, 40683, 40684, 40685, 40686, 40687, 40688, 40689, 40690 were selected from the ORFs having the nucleotide sequence represented by SEQ ID NO: 3143.

The probes having the nucleotide sequence represented by SEQ ID NOS: 40691, 40692, 40693, 40694, 40695, 40696, 40697, 40698, 40699, 40700, 40701 were selected from the ORFs having the nucleotide sequence represented by SEQ ID NO: 3144.

The probes having the nucleotide sequence represented by SEQ ID NOS: 40702, 40703, 40704, 40705, 40706, 40707, 40708, 40709, 40710, 40711, 40712 were selected from the ORFs having the nucleotide sequence represented by SEQ ID NO: 3145.

The probes having the nucleotide sequence represented by SEQ ID NOS: 40713, 40714, 40715, 40716, 40717, 40718, 40719, 40720, 40721, 40722, 40723 were selected from the ORFs having the nucleotide sequence represented by SEQ ID NO: 3146.

The probes having the nucleotide sequence represented by SEQ ID NOS: 40724, 40725, 40726, 40727, 40728, 40729, 40730, 40731, 40732, 40733, 40734 were selected from the ORFs having the nucleotide sequence represented by SEQ ID NO: 3147.

The probes having the nucleotide sequence represented by SEQ ID NOS: 40735, 40736, 40737, 40738, 40739, 40740, 40741, 40742, 40743, 40744, 40745 were selected from the ORFs having the nucleotide sequence represented by SEQ ID NO: 3148.

The probes having the nucleotide sequence represented by SEQ ID NOS: 40746, 40747, 40748, 40749, 40750, 40751, 40752, 40753, 40754, 40755, 40756 were selected from the ORFs having the nucleotide sequence represented by SEQ ID NO: 3149.

The probes having the nucleotide sequence represented by SEQ ID NOS: 40757, 40758, 40759, 40760, 40761, 40762, 40763, 40764, 40765, 40766, 40767 were selected from the ORFs having the nucleotide sequence represented by SEQ ID NO: 3150.

The probes having the nucleotide sequence represented by SEQ ID NOS: 40768, 40769, 40770, 40771, 40772, 40773, 40774, 40775, 40776, 40777, 40778 were selected from the ORFs having the nucleotide sequence represented by SEQ ID NO: 3151.

The probes having the nucleotide sequence represented by SEQ ID NOS: 40779, 40780, 40781, 40782, 40783, 40784, 40785, 40786, 40787, 40788, 40789 were selected from the ORFs having the nucleotide sequence represented by SEQ ID NO: 3152.

The probes having the nucleotide sequence represented by SEQ ID NOS: 40790, 40791, 40792, 40793, 40794, 40795, 40796, 40797, 40798, 40799, 40800 were selected from the ORFs having the nucleotide sequence represented by SEQ ID NO: 3153.

The probes having the nucleotide sequence represented by SEQ ID NOS: 40801, 40802, 40803, 40804, 40805, 40806, 40807, 40808, 40809, 40810, 40811 were selected from the ORFs having the nucleotide sequence represented by SEQ ID NO: 3154.

The probes having the nucleotide sequence represented by SEQ ID NOS: 40812, 40813, 40814, 40815, 40816, 40817, 40818, 40819, 40820, 40821, 40822 were selected from the ORFs having the nucleotide sequence represented by SEQ ID NO: 3155.

The probes having the nucleotide sequence represented by SEQ ID NOS: 40823, 40824, 40825, 40826, 40827, 40828, 40829, 40830, 40831, 40832, 40833 were selected from the ORFs having the nucleotide sequence represented by SEQ ID NO: 3156.

The probes having the nucleotide sequence represented by SEQ ID NOS: 40834, 40835, 40836, 40837, 40838, 40839, 40840, 40841, 40842, 40843, 40844 were selected from the ORFs having the nucleotide sequence represented by SEQ ID NO: 3157.

The probes having the nucleotide sequence represented by SEQ ID NOS: 40845, 40846, 40847, 40848, 40849, 40850, 40851, 40852, 40853, 40854, 40855 were selected from the ORFs having the nucleotide sequence represented by SEQ ID NO: 3158.

The probes having the nucleotide sequence represented by SEQ ID NOS: 40856, 40857, 40858, 40859, 40860, 40861, 40862, 40863, 40864, 40865, 40866 were selected from the ORFs having the nucleotide sequence represented by SEQ ID NO: 3159.

The probes having the nucleotide sequence represented by SEQ ID NOS: 40867, 40868, 40869, 40870, 40871, 40872, 40873, 40874, 40875, 40876, 40877 were selected from the ORFs having the nucleotide sequence represented by SEQ ID NO: 3160.

The probes having the nucleotide sequence represented by SEQ ID NOS: 40878, 40879, 40880, 40881, 40882, 40883, 40884, 40885, 40886, 40887, 40888 were selected from the ORFs having the nucleotide sequence represented by SEQ ID NO: 3161.

The probes having the nucleotide sequence represented by SEQ ID NOS: 40889, 40890, 40891, 40892, 40893, 40894, 40895, 40896, 40897, 40898, 40899 were selected from the ORFs having the nucleotide sequence represented by SEQ ID NO: 3162.

The probes having the nucleotide sequence represented by SEQ ID NOS: 40900, 40901, 40902, 40903, 40904, 40905, 40906, 40907, 40908, 40909, 40910 were selected from the ORFs having the nucleotide sequence represented by SEQ ID NO: 3163.

The probes having the nucleotide sequence represented by SEQ ID NOS: 40911, 40912, 40913, 40914, 40915, 40916, 40917, 40918, 40919, 40920, 40921 were selected from the ORFs having the nucleotide sequence represented by SEQ ID NO: 3164.

The probes having the nucleotide sequence represented by SEQ ID NOS: 40922, 40923, 40924, 40925, 40926, 40927, 40928, 40929, 40930, 40931, 40932 were selected from the ORFs having the nucleotide sequence represented by SEQ ID NO: 3165.

The probes having the nucleotide sequence represented by SEQ ID NOS: 40933, 40934, 40935, 40936, 40937, 40938, 40939, 40940, 40941, 40942, 40943 were selected from the ORFs having the nucleotide sequence represented by SEQ ID NO: 3166.

The probes having the nucleotide sequence represented by SEQ ID NOS: 40944, 40945, 40946, 40947, 40948, 40949, 40950, 40951, 40952, 40953, 40954 were selected from the ORFs having the nucleotide sequence represented by SEQ ID NO: 3167.

The probes having the nucleotide sequence represented by SEQ ID NOS: 40955, 40956, 40957, 40958, 40959, 40960, 40961, 40962, 40963, 40964, 40965 were selected from the ORFs having the nucleotide sequence represented by SEQ ID NO: 3168.

The probes having the nucleotide sequence represented by SEQ ID NOS: 40966, 40967, 40968, 40969, 40970, 40971, 40972, 40973, 40974, 40975, 40976 were selected from the ORFs having the nucleotide sequence represented by SEQ ID NO: 3169.

The probes having the nucleotide sequence represented by SEQ ID NOS: 40977, 40978, 40979, 40980, 40981, 40982, 40983, 40984, 40985, 40986, 40987 were selected from the ORFs having the nucleotide sequence represented by SEQ ID NO: 3170.

The probes having the nucleotide sequence represented by SEQ ID NOS: 40988, 40989, 40990, 40991, 40992, 40993, 40994, 40995, 40996, 40997, 40998 were selected from the ORFs having the nucleotide sequence represented by SEQ ID NO: 3171.

The probes having the nucleotide sequence represented by SEQ ID NOS: 40999, 41000, 41001, 41002, 41003, 41004, 41005, 41006, 41007, 41008, 41009 were selected from the ORFs having the nucleotide sequence represented by SEQ ID NO: 3172.

The probes having the nucleotide sequence represented by SEQ ID NOS: 41010, 41011, 41012, 41013, 41014, 41015, 41016, 41017, 41018, 41019, 41020 were selected from the ORFs having the nucleotide sequence represented by SEQ ID NO: 3173.

The probes having the nucleotide sequence represented by SEQ ID NOS: 41021, 41022, 41023, 41024, 41025, 41026, 41027, 41028, 41029, 41030, 41031 were selected from the ORFs having the nucleotide sequence represented by SEQ ID NO: 3174.

The probes having the nucleotide sequence represented by SEQ ID NOS: 41032, 41033, 41034, 41035, 41036, 41037, 41038, 41039, 41040, 41041, 41042 were selected from the ORFs having the nucleotide sequence represented by SEQ ID NO: 3175.

The probes having the nucleotide sequence represented by SEQ ID NOS: 41043, 41044, 41045, 41046, 41047, 41048, 41049, 41050, 41051, 41052, 41053 were selected from the ORFs having the nucleotide sequence represented by SEQ ID NO: 3176.

The probes having the nucleotide sequence represented by SEQ ID NOS: 41054, 41055, 41056, 41057, 41058, 41059, 41060, 41061, 41062, 41063, 41064 were selected from the ORFs having the nucleotide sequence represented by SEQ ID NO: 3177.

The probes having the nucleotide sequence represented by SEQ ID NOS: 41065, 41066, 41067, 41068, 41069, 41070, 41071, 41072, 41073, 41074, 41075 were selected from the ORFs having the nucleotide sequence represented by SEQ ID NO: 3178.

The probes having the nucleotide sequence represented by SEQ ID NOS: 41076, 41077, 41078, 41079, 41080, 41081, 41082, 41083, 41084, 41085, 41086 were selected from the ORFs having the nucleotide sequence represented by SEQ ID NO: 3179.

The probes having the nucleotide sequence represented by SEQ ID NOS: 41087, 41088, 41089, 41090, 41091, 41092, 41093, 41094, 41095, 41096, 41097 were selected from the ORFs having the nucleotide sequence represented by SEQ ID NO: 3180.

The probes having the nucleotide sequence represented by SEQ ID NOS: 41098, 41099, 41100, 41101, 41102, 41103, 41104, 41105, 41106, 41107, 41108 were selected from the ORFs having the nucleotide sequence represented by SEQ ID NO: 3181.

The probes having the nucleotide sequence represented by SEQ ID NOS: 41109, 41110, 41111, 41112, 41113, 41114, 41115, 41116, 41117, 41118, 41119 were selected from the ORFs having the nucleotide sequence represented by SEQ ID NO: 3182.

The probes having the nucleotide sequence represented by SEQ ID NOS: 41120, 41121, 41122, 41123, 41124, 41125, 41126, 41127, 41128, 41129, 41130 were selected from the ORFs having the nucleotide sequence represented by SEQ ID NO: 3183.

The probes having the nucleotide sequence represented by SEQ ID NOS: 41131, 41132, 41133, 41134, 41135, 41136, 41137, 41138, 41139, 41140, 41141 were selected from the ORFs having the nucleotide sequence represented by SEQ ID NO: 3184.

The probes having the nucleotide sequence represented by SEQ ID NOS: 41142, 41143, 41144, 41145, 41146, 41147, 41148, 41149, 41150, 41151, 41152 were selected from the ORFs having the nucleotide sequence represented by SEQ ID NO: 3185.

The probes having the nucleotide sequence represented by SEQ ID NOS: 41153, 41154, 41155, 41156, 41157, 41158, 41159, 41160, 41161, 41162, 41163 were selected from the ORFs having the nucleotide sequence represented by SEQ ID NO: 3186.

The probes having the nucleotide sequence represented by SEQ ID NOS: 41164, 41165, 41166, 41167, 41168, 41169, 41170, 41171, 41172, 41173, 41174 were selected from the ORFs having the nucleotide sequence represented by SEQ ID NO: 3187.

The probes having the nucleotide sequence represented by SEQ ID NOS: 41175, 41176, 41177, 41178, 41179, 41180, 41181, 41182, 41183, 41184, 41185 were selected from the ORFs having the nucleotide sequence represented by SEQ ID NO: 3188.

The probes having the nucleotide sequence represented by SEQ ID NOS: 41186, 41187, 41188, 41189, 41190, 41191, 41192, 41193, 41194, 41195, 41196 were selected from the ORFs having the nucleotide sequence represented by SEQ ID NO: 3189.

The probes having the nucleotide sequence represented by SEQ ID NOS: 41197, 41198, 41199, 41200, 41201, 41202, 41203, 41204, 41205, 41206, 41207 were selected from the ORFs having the nucleotide sequence represented by SEQ ID NO: 3190.

The probes having the nucleotide sequence represented by SEQ ID NOS: 41208, 41209, 41210, 41211, 41212, 41213, 41214, 41215, 41216, 41217, 41218 were selected from the ORFs having the nucleotide sequence represented by SEQ ID NO: 3191.

The probes having the nucleotide sequence represented by SEQ ID NOS: 41219, 41220, 41221, 41222, 41223, 41224, 41225, 41226, 41227, 41228, 41229 were selected from the ORFs having the nucleotide sequence represented by SEQ ID NO: 3192.

The probes having the nucleotide sequence represented by SEQ ID NOS: 41230, 41231, 41232, 41233, 41234, 41235, 41236, 41237, 41238, 41239, 41240 were selected from the ORFs having the nucleotide sequence represented by SEQ ID NO: 3193.

The probes having the nucleotide sequence represented by SEQ ID NOS: 41241, 41242, 41243, 41244, 41245, 41246, 41247, 41248, 41249, 41250, 41251 were selected from the ORFs having the nucleotide sequence represented by SEQ ID NO: 3194.

The probes having the nucleotide sequence represented by SEQ ID NOS: 41252, 41253, 41254, 41255, 41256, 41257, 41258, 41259, 41260, 41261, 41262 were selected from the ORFs having the nucleotide sequence represented by SEQ ID NO: 3195.

The probes having the nucleotide sequence represented by SEQ ID NOS: 41263, 41264, 41265, 41266, 41267, 41268, 41269, 41270, 41271, 41272, 41273 were selected from the ORFs having the nucleotide sequence represented by SEQ ID NO: 3196.

The probes having the nucleotide sequence represented by SEQ ID NOS: 41274, 41275, 41276, 41277, 41278, 41279, 41280, 41281, 41282, 41283, 41284 were selected from the ORFs having the nucleotide sequence represented by SEQ ID NO: 3197.

The probes having the nucleotide sequence represented by SEQ ID NOS: 41285, 41286, 41287, 41288, 41289, 41290, 41291, 41292, 41293, 41294, 41295 were selected from the ORFs having the nucleotide sequence represented by SEQ ID NO: 3198.

The probes having the nucleotide sequence represented by SEQ ID NOS: 41296, 41297, 41298, 41299, 41300, 41301, 41302, 41303, 41304, 41305, 41306 were selected from the ORFs having the nucleotide sequence represented by SEQ ID NO: 3199.

The probes having the nucleotide sequence represented by SEQ ID NOS: 41307, 41308, 41309, 41310, 41311, 41312, 41313, 41314, 41315, 41316, 41317 were selected from the ORFs having the nucleotide sequence represented by SEQ ID NO: 3200.

The probes having the nucleotide sequence represented by SEQ ID NOS: 41318, 41319, 41320, 41321, 41322, 41323, 41324, 41325, 41326, 41327, 41328 were selected from the ORFs having the nucleotide sequence represented by SEQ ID NO: 3201.

The probes having the nucleotide sequence represented by SEQ ID NOS: 41329, 41330, 41331, 41332, 41333, 41334, 41335, 41336, 41337, 41338, 41339 were selected from the ORFs having the nucleotide sequence represented by SEQ ID NO: 3202.

The probes having the nucleotide sequence represented by SEQ ID NOS: 41340, 41341, 41342, 41343, 41344, 41345, 41346, 41347, 41348, 41349, 41350 were selected from the ORFs having the nucleotide sequence represented by SEQ ID NO: 3203.

The probes having the nucleotide sequence represented by SEQ ID NOS: 41351, 41352, 41353, 41354, 41355, 41356, 41357, 41358, 41359, 41360, 41361 were selected from the ORFs having the nucleotide sequence represented by SEQ ID NO: 3204.

The probes having the nucleotide sequence represented by SEQ ID NOS: 41362, 41363, 41364, 41365, 41366, 41367, 41368, 41369, 41370, 41371, 41372 were selected from the ORFs having the nucleotide sequence represented by SEQ ID NO: 3205.

The probes having the nucleotide sequence represented by SEQ ID NOS: 41373, 41374, 41375, 41376, 41377, 41378, 41379, 41380, 41381, 41382, 41383 were selected from the ORFs having the nucleotide sequence represented by SEQ ID NO: 3206.

The probes having the nucleotide sequence represented by SEQ ID NOS: 41384, 41385, 41386, 41387, 41388, 41389, 41390, 41391, 41392, 41393, 41394 were selected from the ORFs having the nucleotide sequence represented by SEQ ID NO: 3207.

The probes having the nucleotide sequence represented by SEQ ID NOS: 41395, 41396, 41397, 41398, 41399, 41400, 41401, 41402, 41403, 41404, 41405 were selected from the ORFs having the nucleotide sequence represented by SEQ ID NO: 3208.

The probes having the nucleotide sequence represented by SEQ ID NOS: 41406, 41407, 41408, 41409, 41410, 41411, 41412, 41413, 41414, 41415, 41416 were selected from the ORFs having the nucleotide sequence represented by SEQ ID NO: 3209.

The probes having the nucleotide sequence represented by SEQ ID NOS: 41417, 41418, 41419, 41420, 41421, 41422, 41423, 41424, 41425, 41426, 41427 were selected from the ORFs having the nucleotide sequence represented by SEQ ID NO: 3210.

The probes having the nucleotide sequence represented by SEQ ID NOS: 41428, 41429, 41430, 41431, 41432, 41433, 41434, 41435, 41436, 41437, 41438 were selected from the ORFs having the nucleotide sequence represented by SEQ ID NO: 3211.

The probes having the nucleotide sequence represented by SEQ ID NOS: 41439, 41440, 41441, 41442, 41443, 41444, 41445, 41446, 41447, 41448, 41449 were selected from the ORFs having the nucleotide sequence represented by SEQ ID NO: 3212.

The probes having the nucleotide sequence represented by SEQ ID NOS: 41450, 41451, 41452, 41453, 41454, 41455, 41456, 41457, 41458, 41459, 41460 were selected from the ORFs having the nucleotide sequence represented by SEQ ID NO: 3213.

The probes having the nucleotide sequence represented by SEQ ID NOS: 41461, 41462, 41463, 41464, 41465, 41466, 41467, 41468, 41469, 41470, 41471 were selected from the ORFs having the nucleotide sequence represented by SEQ ID NO: 3214.

The probes having the nucleotide sequence represented by SEQ ID NOS: 41472, 41473, 41474, 41475, 41476, 41477, 41478, 41479, 41480, 41481, 41482 were selected from the ORFs having the nucleotide sequence represented by SEQ ID NO: 3215.

The probes having the nucleotide sequence represented by SEQ ID NOS: 41483, 41484, 41485, 41486, 41487, 41488, 41489, 41490, 41491, 41492, 41493 were selected from the ORFs having the nucleotide sequence represented by SEQ ID NO: 3216.

The probes having the nucleotide sequence represented by SEQ ID NOS: 41494, 41495, 41496, 41497, 41498, 41499, 41500, 41501, 41502, 41503, 41504 were selected from the ORFs having the nucleotide sequence represented by SEQ ID NO: 3217.

The probes having the nucleotide sequence represented by SEQ ID NOS: 41505, 41506, 41507, 41508, 41509, 41510, 41511, 41512, 41513, 41514, 41515 were selected from the ORFs having the nucleotide sequence represented by SEQ ID NO: 3218.

The probes having the nucleotide sequence represented by SEQ ID NOS: 41516, 41517, 41518, 41519, 41520, 41521, 41522, 41523, 41524, 41525, 41526 were selected from the ORFs having the nucleotide sequence represented by SEQ ID NO: 3219.

The probes having the nucleotide sequence represented by SEQ ID NOS: 41527, 41528, 41529, 41530, 41531, 41532, 41533, 41534, 41535, 41536, 41537 were selected from the ORFs having the nucleotide sequence represented by SEQ ID NO: 3220.

The probes having the nucleotide sequence represented by SEQ ID NOS: 41538, 41539, 41540, 41541, 41542, 41543, 41544, 41545, 41546, 41547, 41548 were selected from the ORFs having the nucleotide sequence represented by SEQ ID NO: 3221.

The probes having the nucleotide sequence represented by SEQ ID NOS: 41549, 41550, 41551, 41552, 41553, 41554, 41555, 41556, 41557, 41558, 41559 were selected from the ORFs having the nucleotide sequence represented by SEQ ID NO: 3222.

The probes having the nucleotide sequence represented by SEQ ID NOS: 41560, 41561, 41562, 41563, 41564, 41565, 41566, 41567, 41568, 41569, 41570 were selected from the ORFs having the nucleotide sequence represented by SEQ ID NO: 3223.

The probes having the nucleotide sequence represented by SEQ ID NOS: 41571, 41572, 41573, 41574, 41575, 41576, 41577, 41578, 41579, 41580, 41581 were selected from the ORFs having the nucleotide sequence represented by SEQ ID NO: 3224.

The probes having the nucleotide sequence represented by SEQ ID NOS: 41582, 41583, 41584, 41585, 41586, 41587, 41588, 41589, 41590, 41591, 41592 were selected from the ORFs having the nucleotide sequence represented by SEQ ID NO: 3225.

The probes having the nucleotide sequence represented by SEQ ID NOS: 41593, 41594, 41595, 41596, 41597, 41598, 41599, 41600, 41601, 41602, 41603 were selected from the ORFs having the nucleotide sequence represented by SEQ ID NO: 3226.

The probes having the nucleotide sequence represented by SEQ ID NOS: 41604, 41605, 41606, 41607, 41608, 41609, 41610, 41611, 41612, 41613, 41614 were selected from the ORFs having the nucleotide sequence represented by SEQ ID NO: 3227.

The probes having the nucleotide sequence represented by SEQ ID NOS: 41615, 41616, 41617, 41618, 41619, 41620, 41621, 41622, 41623, 41624, 41625 were selected from the ORFs having the nucleotide sequence represented by SEQ ID NO: 3228.

The probes having the nucleotide sequence represented by SEQ ID NOS: 41626, 41627, 41628, 41629, 41630, 41631, 41632, 41633, 41634, 41635, 41636 were selected from the ORFs having the nucleotide sequence represented by SEQ ID NO: 3229.

The probes having the nucleotide sequence represented by SEQ ID NOS: 41637, 41638, 41639, 41640, 41641, 41642, 41643, 41644, 41645, 41646, 41647 were selected from the ORFs having the nucleotide sequence represented by SEQ ID NO: 3230.

The probes having the nucleotide sequence represented by SEQ ID NOS: 41648, 41649, 41650, 41651, 41652, 41653, 41654, 41655, 41656, 41657, 41658 were selected from the ORFs having the nucleotide sequence represented by SEQ ID NO: 3231.

The probes having the nucleotide sequence represented by SEQ ID NOS: 41659, 41660, 41661, 41662, 41663, 41664, 41665, 41666, 41667, 41668, 41669 were selected from the ORFs having the nucleotide sequence represented by SEQ ID NO: 3232.

The probes having the nucleotide sequence represented by SEQ ID NOS: 41670, 41671, 41672, 41673, 41674, 41675, 41676, 41677, 41678, 41679, 41680 were selected from the ORFs having the nucleotide sequence represented by SEQ ID NO: 3233.

The probes having the nucleotide sequence represented by SEQ ID NOS: 41681, 41682, 41683, 41684, 41685, 41686, 41687, 41688, 41689, 41690, 41691 were selected from the ORFs having the nucleotide sequence represented by SEQ ID NO: 3234.

The probes having the nucleotide sequence represented by SEQ ID NOS: 41692, 41693, 41694, 41695, 41696, 41697, 41698, 41699, 41700, 41701, 41702 were selected from the ORFs having the nucleotide sequence represented by SEQ ID NO: 3235.

The probes having the nucleotide sequence represented by SEQ ID NOS: 41703, 41704, 41705, 41706, 41707, 41708, 41709, 41710, 41711, 41712, 41713 were selected from the ORFs having the nucleotide sequence represented by SEQ ID NO: 3236.

The probes having the nucleotide sequence represented by SEQ ID NOS: 41714, 41715, 41716, 41717, 41718, 41719, 41720, 41721, 41722, 41723, 41724 were selected from the ORFs having the nucleotide sequence represented by SEQ ID NO: 3237.

The probes having the nucleotide sequence represented by SEQ ID NOS: 41725, 41726, 41727, 41728, 41729, 41730, 41731, 41732, 41733, 41734, 41735 were selected from the ORFs having the nucleotide sequence represented by SEQ ID NO: 3238.

The probes having the nucleotide sequence represented by SEQ ID NOS: 41736, 41737, 41738, 41739, 41740, 41741, 41742, 41743, 41744, 41745, 41746 were selected from the ORFs having the nucleotide sequence represented by SEQ ID NO: 3239.

The probes having the nucleotide sequence represented by SEQ ID NOS: 41747, 41748, 41749, 41750, 41751, 41752, 41753, 41754, 41755, 41756, 41757 were selected from the ORFs having the nucleotide sequence represented by SEQ ID NO: 3240.

The probes having the nucleotide sequence represented by SEQ ID NOS: 41758, 41759, 41760, 41761, 41762, 41763, 41764, 41765, 41766, 41767, 41768 were selected from the ORFs having the nucleotide sequence represented by SEQ ID NO: 3241.

The probes having the nucleotide sequence represented by SEQ ID NOS: 41769, 41770, 41771, 41772, 41773, 41774, 41775, 41776, 41777, 41778, 41779 were selected from the ORFs having the nucleotide sequence represented by SEQ ID NO: 3242.

The probes having the nucleotide sequence represented by SEQ ID NOS: 41780, 41781, 41782, 41783, 41784, 41785, 41786, 41787, 41788, 41789, 41790 were selected from the ORFs having the nucleotide sequence represented by SEQ ID NO: 3243.

The probes having the nucleotide sequence represented by SEQ ID NOS: 41791, 41792, 41793, 41794, 41795, 41796, 41797, 41798, 41799, 41800, 41801 were selected from the ORFs having the nucleotide sequence represented by SEQ ID NO: 3244.

The probes having the nucleotide sequence represented by SEQ ID NOS: 41802, 41803, 41804, 41805, 41806, 41807, 41808, 41809, 41810, 41811, 41812 were selected from the ORFs having the nucleotide sequence represented by SEQ ID NO: 3245.

The probes having the nucleotide sequence represented by SEQ ID NOS: 41813, 41814, 41815, 41816, 41817, 41818, 41819, 41820, 41821, 41822, 41823 were selected from the ORFs having the nucleotide sequence represented by SEQ ID NO: 3246.

The probes having the nucleotide sequence represented by SEQ ID NOS: 41824, 41825, 41826, 41827, 41828, 41829, 41830, 41831, 41832, 41833, 41834 were selected from the ORFs having the nucleotide sequence represented by SEQ ID NO: 3247.

The probes having the nucleotide sequence represented by SEQ ID NOS: 41835, 41836, 41837, 41838, 41839, 41840, 41841, 41842, 41843, 41844, 41845 were selected from the ORFs having the nucleotide sequence represented by SEQ ID NO: 3248.

The probes having the nucleotide sequence represented by SEQ ID NOS: 41846, 41847, 41848, 41849, 41850, 41851, 41852, 41853, 41854, 41855, 41856 were selected from the ORFs having the nucleotide sequence represented by SEQ ID NO: 3249.

The probes having the nucleotide sequence represented by SEQ ID NOS: 41857, 41858, 41859, 41860, 41861, 41862, 41863, 41864, 41865, 41866, 41867 were selected from the ORFs having the nucleotide sequence represented by SEQ ID NO: 3250.

The probes having the nucleotide sequence represented by SEQ ID NOS: 41868, 41869, 41870, 41871, 41872, 41873, 41874, 41875, 41876, 41877, 41878 were selected from the ORFs having the nucleotide sequence represented by SEQ ID NO: 3251.

The probes having the nucleotide sequence represented by SEQ ID NOS: 41879, 41880, 41881, 41882, 41883, 41884, 41885, 41886, 41887, 41888, 41889 were selected from the ORFs having the nucleotide sequence represented by SEQ ID NO: 3252.

The probes having the nucleotide sequence represented by SEQ ID NOS: 41890, 41891, 41892, 41893, 41894, 41895, 41896, 41897, 41898, 41899, 41900 were selected from the ORFs having the nucleotide sequence represented by SEQ ID NO: 3253.

The probes having the nucleotide sequence represented by SEQ ID NOS: 41901, 41902, 41903, 41904, 41905, 41906, 41907, 41908, 41909, 41910, 41911 were selected from the ORFs having the nucleotide sequence represented by SEQ ID NO: 3254.

The probes having the nucleotide sequence represented by SEQ ID NOS: 41912, 41913, 41914, 41915, 41916, 41917, 41918, 41919, 41920, 41921, 41922 were selected from the ORFs having the nucleotide sequence represented by SEQ ID NO: 3255.

The probes having the nucleotide sequence represented by SEQ ID NOS: 41923, 41924, 41925, 41926, 41927, 41928, 41929, 41930, 41931, 41932, 41933 were selected from the ORFs having the nucleotide sequence represented by SEQ ID NO: 3256.

The probes having the nucleotide sequence represented by SEQ ID NOS: 41934, 41935, 41936, 41937, 41938, 41939, 41940, 41941, 41942, 41943, 41944 were selected from the ORFs having the nucleotide sequence represented by SEQ ID NO: 3257.

The probes having the nucleotide sequence represented by SEQ ID NOS: 41945, 41946, 41947, 41948, 41949, 41950, 41951, 41952, 41953, 41954, 41955 were selected from the ORFs having the nucleotide sequence represented by SEQ ID NO: 3258.

The probes having the nucleotide sequence represented by SEQ ID NOS: 41956, 41957, 41958, 41959, 41960, 41961, 41962, 41963, 41964, 41965, 41966 were selected from the ORFs having the nucleotide sequence represented by SEQ ID NO: 3259.

The probes having the nucleotide sequence represented by SEQ ID NOS: 41967, 41968, 41969, 41970, 41971, 41972, 41973, 41974, 41975, 41976, 41977 were selected from the ORFs having the nucleotide sequence represented by SEQ ID NO: 3260.

The probes having the nucleotide sequence represented by SEQ ID NOS: 41978, 41979, 41980, 41981, 41982, 41983, 41984, 41985, 41986, 41987, 41988 were selected from the ORFs having the nucleotide sequence represented by SEQ ID NO: 3261.

The probes having the nucleotide sequence represented by SEQ ID NOS: 41989, 41990, 41991, 41992, 41993, 41994, 41995, 41996, 41997, 41998, 41999 were selected from the ORFs having the nucleotide sequence represented by SEQ ID NO: 3262.

The probes having the nucleotide sequence represented by SEQ ID NOS: 42000, 42001, 42002, 42003, 42004, 42005, 42006, 42007, 42008, 42009, 42010 were selected from the ORFs having the nucleotide sequence represented by SEQ ID NO: 3263.

The probes having the nucleotide sequence represented by SEQ ID NOS: 42011, 42012, 42013, 42014, 42015, 42016, 42017, 42018, 42019, 42020, 42021 were selected from the ORFs having the nucleotide sequence represented by SEQ ID NO: 3264.

The probes having the nucleotide sequence represented by SEQ ID NOS: 42022, 42023, 42024, 42025, 42026, 42027, 42028, 42029, 42030, 42031, 42032 were selected from the ORFs having the nucleotide sequence represented by SEQ ID NO: 3265.

The probes having the nucleotide sequence represented by SEQ ID NOS: 42033, 42034, 42035, 42036, 42037, 42038, 42039, 42040, 42041, 42042, 42043 were selected from the ORFs having the nucleotide sequence represented by SEQ ID NO: 3266.

The probes having the nucleotide sequence represented by SEQ ID NOS: 42044, 42045, 42046, 42047, 42048, 42049, 42050, 42051, 42052, 42053, 42054 were selected from the ORFs having the nucleotide sequence represented by SEQ ID NO: 3267.

The probes having the nucleotide sequence represented by SEQ ID NOS: 42055, 42056, 42057, 42058, 42059, 42060, 42061, 42062, 42063, 42064, 42065 were selected from the ORFs having the nucleotide sequence represented by SEQ ID NO: 3268.

The probes having the nucleotide sequence represented by SEQ ID NOS: 42066, 42067, 42068, 42069, 42070, 42071, 42072, 42073, 42074, 42075, 42076 were selected from the ORFs having the nucleotide sequence represented by SEQ ID NO: 3269.

The probes having the nucleotide sequence represented by SEQ ID NOS: 42077, 42078, 42079, 42080, 42081, 42082, 42083, 42084, 42085, 42086, 42087 were selected from the ORFs having the nucleotide sequence represented by SEQ ID NO: 3270.

The probes having the nucleotide sequence represented by SEQ ID NOS: 42088, 42089, 42090, 42091, 42092, 42093, 42094, 42095, 42096, 42097, 42098 were selected from the ORFs having the nucleotide sequence represented by SEQ ID NO: 3271.

The probes having the nucleotide sequence represented by SEQ ID NOS: 42099, 42100, 42101, 42102, 42103, 42104, 42105, 42106, 42107, 42108, 42109 were selected from the ORFs having the nucleotide sequence represented by SEQ ID NO: 3272.

The probes having the nucleotide sequence represented by SEQ ID NOS: 42110, 42111, 42112, 42113, 42114, 42115, 42116, 42117, 42118, 42119, 42120 were selected from the ORFs having the nucleotide sequence represented by SEQ ID NO: 3273.

The probes having the nucleotide sequence represented by SEQ ID NOS: 42121, 42122, 42123, 42124, 42125, 42126, 42127, 42128, 42129, 42130, 42131 were selected from the ORFs having the nucleotide sequence represented by SEQ ID NO: 3274.

The probes having the nucleotide sequence represented by SEQ ID NOS: 42132, 42133, 42134, 42135, 42136, 42137, 42138, 42139, 42140, 42141, 42142 were selected from the ORFs having the nucleotide sequence represented by SEQ ID NO: 3275.

The probes having the nucleotide sequence represented by SEQ ID NOS: 42143, 42144, 42145, 42146, 42147, 42148, 42149, 42150, 42151, 42152, 42153 were selected from the ORFs having the nucleotide sequence represented by SEQ ID NO: 3276.

The probes having the nucleotide sequence represented by SEQ ID NOS: 42154, 42155, 42156, 42157, 42158, 42159, 42160, 42161, 42162, 42163, 42164 were selected from the ORFs having the nucleotide sequence represented by SEQ ID NO: 3277.

The probes having the nucleotide sequence represented by SEQ ID NOS: 42165, 42166, 42167, 42168, 42169, 42170, 42171, 42172, 42173, 42174, 42175 were selected from the ORFs having the nucleotide sequence represented by SEQ ID NO: 3278.

The probes having the nucleotide sequence represented by SEQ ID NOS: 42176, 42177, 42178, 42179, 42180, 42181, 42182, 42183, 42184, 42185, 42186 were selected from the ORFs having the nucleotide sequence represented by SEQ ID NO: 3279.

The probes having the nucleotide sequence represented by SEQ ID NOS: 42187, 42188, 42189, 42190, 42191, 42192, 42193, 42194, 42195, 42196, 42197 were selected from the ORFs having the nucleotide sequence represented by SEQ ID NO: 3280.

The probes having the nucleotide sequence represented by SEQ ID NOS: 42198, 42199, 42200, 42201, 42202, 42203, 42204, 42205, 42206, 42207, 42208 were selected from the ORFs having the nucleotide sequence represented by SEQ ID NO: 3281.

The probes having the nucleotide sequence represented by SEQ ID NOS: 42209, 42210, 42211, 42212, 42213, 42214, 42215, 42216, 42217, 42218, 42219 were selected from the ORFs having the nucleotide sequence represented by SEQ ID NO: 3282.

The probes having the nucleotide sequence represented by SEQ ID NOS: 42220, 42221, 42222, 42223, 42224, 42225, 42226, 42227, 42228, 42229, 42230 were selected from the ORFs having the nucleotide sequence represented by SEQ ID NO: 3283.

The probes having the nucleotide sequence represented by SEQ ID NOS: 42231, 42232, 42233, 42234, 42235, 42236, 42237, 42238, 42239, 42240, 42241 were selected from the ORFs having the nucleotide sequence represented by SEQ ID NO: 3284.

The probes having the nucleotide sequence represented by SEQ ID NOS: 42242, 42243, 42244, 42245, 42246, 42247, 42248, 42249, 42250, 42251, 42252 were selected from the ORFs having the nucleotide sequence represented by SEQ ID NO: 3285.

The probes having the nucleotide sequence represented by SEQ ID NOS: 42253, 42254, 42255, 42256, 42257, 42258, 42259, 42260, 42261, 42262, 42263 were selected from the ORFs having the nucleotide sequence represented by SEQ ID NO: 3286.

The probes having the nucleotide sequence represented by SEQ ID NOS: 42264, 42265, 42266, 42267, 42268, 42269, 42270, 42271, 42272, 42273, 42274 were selected from the ORFs having the nucleotide sequence represented by SEQ ID NO: 3287.

The probes having the nucleotide sequence represented by SEQ ID NOS: 42275, 42276, 42277, 42278, 42279, 42280, 42281, 42282, 42283, 42284, 42285 were selected from the ORFs having the nucleotide sequence represented by SEQ ID NO: 3288.

The probes having the nucleotide sequence represented by SEQ ID NOS: 42286, 42287, 42288, 42289, 42290, 42291, 42292, 42293, 42294, 42295, 42296 were selected from the ORFs having the nucleotide sequence represented by SEQ ID NO: 3289.

The probes having the nucleotide sequence represented by SEQ ID NOS: 42297, 42298, 42299, 42300, 42301, 42302, 42303, 42304, 42305, 42306, 42307 were selected from the ORFs having the nucleotide sequence represented by SEQ ID NO: 3290.

The probes having the nucleotide sequence represented by SEQ ID NOS: 42308, 42309, 42310, 42311, 42312, 42313, 42314, 42315, 42316, 42317, 42318 were selected from the ORFs having the nucleotide sequence represented by SEQ ID NO: 3291.

The probes having the nucleotide sequence represented by SEQ ID NOS: 42319, 42320, 42321, 42322, 42323, 42324, 42325, 42326, 42327, 42328, 42329 were selected from the ORFs having the nucleotide sequence represented by SEQ ID NO: 3292.

The probes having the nucleotide sequence represented by SEQ ID NOS: 42330, 42331, 42332, 42333, 42334, 42335, 42336, 42337, 42338, 42339, 42340 were selected from the ORFs having the nucleotide sequence represented by SEQ ID NO: 3293.

The probes having the nucleotide sequence represented by SEQ ID NOS: 42341, 42342, 42343, 42344, 42345, 42346, 42347, 42348, 42349, 42350, 42351 were selected from the ORFs having the nucleotide sequence represented by SEQ ID NO: 3294.

The probes having the nucleotide sequence represented by SEQ ID NOS: 42352, 42353, 42354, 42355, 42356, 42357, 42358, 42359, 42360, 42361, 42362 were selected from the ORFs having the nucleotide sequence represented by SEQ ID NO: 3295.

The probes having the nucleotide sequence represented by SEQ ID NOS: 42363, 42364, 42365, 42366, 42367, 42368, 42369, 42370, 42371, 42372, 42373 were selected from the ORFs having the nucleotide sequence represented by SEQ ID NO: 3296.

The probes having the nucleotide sequence represented by SEQ ID NOS: 42374, 42375, 42376, 42377, 42378, 42379, 42380, 42381, 42382, 42383, 42384 were selected from the ORFs having the nucleotide sequence represented by SEQ ID NO: 3297.

The probes having the nucleotide sequence represented by SEQ ID NOS: 42385, 42386, 42387, 42388, 42389, 42390, 42391, 42392, 42393, 42394, 42395 were selected from the ORFs having the nucleotide sequence represented by SEQ ID NO: 3298.

The probes having the nucleotide sequence represented by SEQ ID NOS: 42396, 42397, 42398, 42399, 42400, 42401, 42402, 42403, 42404, 42405, 42406 were selected from the ORFs having the nucleotide sequence represented by SEQ ID NO: 3299.

The probes having the nucleotide sequence represented by SEQ ID NOS: 42407, 42408, 42409, 42410, 42411, 42412, 42413, 42414, 42415, 42416, 42417 were selected from the ORFs having the nucleotide sequence represented by SEQ ID NO: 3300.

The probes having the nucleotide sequence represented by SEQ ID NOS: 42418, 42419, 42420, 42421, 42422, 42423, 42424, 42425, 42426, 42427, 42428 were selected from the ORFs having the nucleotide sequence represented by SEQ ID NO: 3301.

The probes having the nucleotide sequence represented by SEQ ID NOS: 42429, 42430, 42431, 42432, 42433, 42434, 42435, 42436, 42437, 42438, 42439 were selected from the ORFs having the nucleotide sequence represented by SEQ ID NO: 3302.

The probes having the nucleotide sequence represented by SEQ ID NOS: 42440, 42441, 42442, 42443, 42444, 42445, 42446, 42447, 42448, 42449, 42450 were selected from the ORFs having the nucleotide sequence represented by SEQ ID NO: 3303.

The probes having the nucleotide sequence represented by SEQ ID NOS: 42451, 42452, 42453, 42454, 42455, 42456, 42457, 42458, 42459, 42460, 42461 were selected from the ORFs having the nucleotide sequence represented by SEQ ID NO: 3304.

The probes having the nucleotide sequence represented by SEQ ID NOS: 42462, 42463, 42464, 42465, 42466, 42467, 42468, 42469, 42470, 42471, 42472 were selected from the ORFs having the nucleotide sequence represented by SEQ ID NO: 3305.

The probes having the nucleotide sequence represented by SEQ ID NOS: 42473, 42474, 42475, 42476, 42477, 42478, 42479, 42480, 42481, 42482, 42483 were selected from the ORFs having the nucleotide sequence represented by SEQ ID NO: 3306.

The probes having the nucleotide sequence represented by SEQ ID NOS: 42484, 42485, 42486, 42487, 42488, 42489, 42490, 42491, 42492, 42493, 42494 were selected from the ORFs having the nucleotide sequence represented by SEQ ID NO: 3307.

The probes having the nucleotide sequence represented by SEQ ID NOS: 42495, 42496, 42497, 42498, 42499, 42500, 42501, 42502, 42503, 42504, 42505 were selected from the ORFs having the nucleotide sequence represented by SEQ ID NO: 3308.

The probes having the nucleotide sequence represented by SEQ ID NOS: 42506, 42507, 42508, 42509, 42510, 42511, 42512, 42513, 42514, 42515, 42516 were selected from the ORFs having the nucleotide sequence represented by SEQ ID NO: 3309.

The probes having the nucleotide sequence represented by SEQ ID NOS: 42517, 42518, 42519, 42520, 42521, 42522, 42523, 42524, 42525, 42526, 42527 were selected from the ORFs having the nucleotide sequence represented by SEQ ID NO: 3310.

The probes having the nucleotide sequence represented by SEQ ID NOS: 42528, 42529, 42530, 42531, 42532, 42533, 42534, 42535, 42536, 42537, 42538 were selected from the ORFs having the nucleotide sequence represented by SEQ ID NO: 3311.

The probes having the nucleotide sequence represented by SEQ ID NOS: 42539, 42540, 42541, 42542, 42543, 42544, 42545, 42546, 42547, 42548, 42549 were selected from the ORFs having the nucleotide sequence represented by SEQ ID NO: 3312.

The probes having the nucleotide sequence represented by SEQ ID NOS: 42550, 42551, 42552, 42553, 42554, 42555, 42556, 42557, 42558, 42559, 42560 were selected from the ORFs having the nucleotide sequence represented by SEQ ID NO: 3313.

The probes having the nucleotide sequence represented by SEQ ID NOS: 42561, 42562, 42563, 42564, 42565, 42566, 42567, 42568, 42569, 42570, 42571 were selected from the ORFs having the nucleotide sequence represented by SEQ ID NO: 3314.

The probes having the nucleotide sequence represented by SEQ ID NOS: 42572, 42573, 42574, 42575, 42576, 42577, 42578, 42579, 42580, 42581, 42582 were selected from the ORFs having the nucleotide sequence represented by SEQ ID NO: 3315.

The probes having the nucleotide sequence represented by SEQ ID NOS: 42583, 42584, 42585, 42586, 42587, 42588, 42589, 42590, 42591, 42592, 42593 were selected from the ORFs having the nucleotide sequence represented by SEQ ID NO: 3316.

The probes having the nucleotide sequence represented by SEQ ID NOS: 42594, 42595, 42596, 42597, 42598, 42599, 42600, 42601, 42602, 42603, 42604 were selected from the ORFs having the nucleotide sequence represented by SEQ ID NO: 3317.

The probes having the nucleotide sequence represented by SEQ ID NOS: 42605, 42606, 42607, 42608, 42609, 42610, 42611, 42612, 42613, 42614, 42615 were selected from the ORFs having the nucleotide sequence represented by SEQ ID NO: 3318.

The probes having the nucleotide sequence represented by SEQ ID NOS: 42616, 42617, 42618, 42619, 42620, 42621, 42622, 42623, 42624, 42625, 42626 were selected from the ORFs having the nucleotide sequence represented by SEQ ID. NO: 3319.

The probes having the nucleotide sequence represented by SEQ ID NOS: 42627, 42628, 42629, 42630, 42631, 42632, 42633, 42634, 42635, 42636, 42637 were selected from the ORFs having the nucleotide sequence represented by SEQ ID NO: 3320.

The probes having the nucleotide sequence represented by SEQ ID NOS: 42638, 42639, 42640, 42641, 42642, 42643, 42644, 42645, 42646, 42647, 42648 were selected from the ORFs having the nucleotide sequence represented by SEQ ID NO: 3321.

The probes having the nucleotide sequence represented by SEQ ID NOS: 42649, 42650, 42651, 42652, 42653, 42654, 42655, 42656, 42657, 42658, 42659 were selected from the ORFs having the nucleotide sequence represented by SEQ ID NO: 3322.

The probes having the nucleotide sequence represented by SEQ ID NOS: 42660, 42661, 42662, 42663, 42664, 42665, 42666, 42667, 42668, 42669, 42670 were selected from the ORFs having the nucleotide sequence represented by SEQ ID NO: 3323.

The probes having the nucleotide sequence represented by SEQ ID NOS: 42671, 42672, 42673, 42674, 42675, 42676, 42677, 42678, 42679, 42680, 42681 were selected from the ORFs having the nucleotide sequence represented by SEQ ID NO: 3324.

The probes having the nucleotide sequence represented by SEQ ID NOS: 42682, 42683, 42684, 42685, 42686, 42687, 42688, 42689, 42690, 42691, 42692 were selected from the ORFs having the nucleotide sequence represented by SEQ ID NO: 3325.

The probes having the nucleotide sequence represented by SEQ ID NOS: 42693, 42694, 42695, 42696, 42697, 42698, 42699, 42700, 42701, 42702, 42703 were selected from the ORFs having the nucleotide sequence represented by SEQ ID NO: 3326.

The probes having the nucleotide sequence represented by SEQ ID NOS: 42704, 42705, 42706, 42707, 42708, 42709, 42710, 42711, 42712, 42713, 42714 were selected from the ORFs having the nucleotide sequence represented by SEQ ID NO: 3327.

The probes having the nucleotide sequence represented by SEQ ID NOS: 42715, 42716, 42717, 42718, 42719, 42720, 42721, 42722, 42723, 42724, 42725 were selected from the ORFs having the nucleotide sequence represented by SEQ ID NO: 3328.

The probes having the nucleotide sequence represented by SEQ ID NOS: 42726, 42727, 42728, 42729, 42730, 42731, 42732, 42733, 42734, 42735, 42736 were selected from the ORFs having the nucleotide sequence represented by SEQ ID NO: 3329.

The probes having the nucleotide sequence represented by SEQ ID NOS: 42737, 42738, 42739, 42740, 42741, 42742, 42743, 42744, 42745, 42746, 42747 were selected from the ORFs having the nucleotide sequence represented by SEQ ID NO: 3330.

The probes having the nucleotide sequence represented by SEQ ID NOS: 42748, 42749, 42750, 42751, 42752, 42753, 42754, 42755, 42756, 42757, 42758 were selected from the ORFs having the nucleotide sequence represented by SEQ ID NO: 3331.

The probes having the nucleotide sequence represented by SEQ ID NOS: 42759, 42760, 42761, 42762, 42763, 42764, 42765, 42766, 42767, 42768, 42769 were selected from the ORFs having the nucleotide sequence represented by SEQ ID NO: 3332.

The probes having the nucleotide sequence represented by SEQ ID NOS: 42770, 42771, 42772, 42773, 42774, 42775, 42776, 42777, 42778, 42779, 42780 were selected from the ORFs having the nucleotide sequence represented by SEQ ID NO: 3333.

The probes having the nucleotide sequence represented by SEQ ID NOS: 42781, 42782, 42783, 42784, 42785, 42786, 42787, 42788, 42789, 42790, 42791 were selected from the ORFs having the nucleotide sequence represented by SEQ ID NO: 3334.

The probes having the nucleotide sequence represented by SEQ ID NOS: 42792, 42793, 42794, 42795, 42796, 42797, 42798, 42799, 42800, 42801, 42802 were selected from the ORFs having the nucleotide sequence represented by SEQ ID NO: 3335.

The probes having the nucleotide sequence represented by SEQ ID NOS: 42803, 42804, 42805, 42806, 42807, 42808, 42809, 42810, 42811, 42812, 42813 were selected from the ORFs having the nucleotide sequence represented by SEQ ID NO: 3336.

The probes having the nucleotide sequence represented by SEQ ID NOS: 42814, 42815, 42816, 42817, 42818, 42819, 42820, 42821, 42822, 42823, 42824 were selected from the ORFs having the nucleotide sequence represented by SEQ ID NO: 3337.

The probes having the nucleotide sequence represented by SEQ ID NOS: 42825, 42826, 42827, 42828, 42829, 42830, 42831, 42832, 42833, 42834, 42835 were selected from the ORFs having the nucleotide sequence represented by SEQ ID NO: 3338.

The probes having the nucleotide sequence represented by SEQ ID NOS: 42836, 42837, 42838, 42839, 42840, 42841, 42842, 42843, 42844, 42845, 42846 were selected from the ORFs having the nucleotide sequence represented by SEQ ID NO: 3339.

The probes having the nucleotide sequence represented by SEQ ID NOS: 42847, 42848, 42849, 42850, 42851, 42852, 42853, 42854, 42855, 42856, 42857 were selected from the ORFs having the nucleotide sequence represented by SEQ ID NO: 3340.

The probes having the nucleotide sequence represented by SEQ ID NOS: 42858, 42859, 42860, 42861, 42862, 42863, 42864, 42865, 42866, 42867, 42868 were selected from the ORFs having the nucleotide sequence represented by SEQ ID NO: 3341.

The probes having the nucleotide sequence represented by SEQ ID NOS: 42869, 42870, 42871, 42872, 42873, 42874, 42875, 42876, 42877, 42878, 42879 were selected from the ORFs having the nucleotide sequence represented by SEQ ID NO: 3342.

The probes having the nucleotide sequence represented by SEQ ID NOS: 42880, 42881, 42882, 42883, 42884, 42885, 42886, 42887, 42888, 42889, 42890 were selected from the ORFs having the nucleotide sequence represented by SEQ ID NO: 3343.

The probes having the nucleotide sequence represented by SEQ ID NOS: 42891, 42892, 42893, 42894, 42895, 42896, 42897, 42898, 42899, 42900, 42901 were selected from the ORFs having the nucleotide sequence represented by SEQ ID NO: 3344.

The probes having the nucleotide sequence represented by SEQ ID NOS: 42902, 42903, 42904, 42905, 42906, 42907, 42908, 42909, 42910, 42911, 42912 were selected from the ORFs having the nucleotide sequence represented by SEQ ID NO: 3345.

The probes having the nucleotide sequence represented by SEQ ID NOS: 42913, 42914, 42915, 42916, 42917, 42918, 42919, 42920, 42921, 42922, 42923 were selected from the ORFs having the nucleotide sequence represented by SEQ ID NO: 3346.

The probes having the nucleotide sequence represented by SEQ ID NOS: 42924, 42925, 42926, 42927, 42928, 42929, 42930, 42931, 42932, 42933, 42934 were selected from the ORFs having the nucleotide sequence represented by SEQ ID NO: 3347.

The probes having the nucleotide sequence represented by SEQ ID NOS: 42935, 42936, 42937, 42938, 42939, 42940, 42941, 42942, 42943, 42944, 42945 were selected from the ORFs having the nucleotide sequence represented by SEQ ID NO: 3348.

The probes having the nucleotide sequence represented by SEQ ID NOS: 42946, 42947, 42948, 42949, 42950, 42951, 42952, 42953, 42954, 42955, 42956 were selected from the ORFs having the nucleotide sequence represented by SEQ ID NO: 3349.

The probes having the nucleotide sequence represented by SEQ ID NOS: 42957, 42958, 42959, 42960, 42961, 42962, 42963, 42964, 42965, 42966, 42967 were selected from the ORFs having the nucleotide sequence represented by SEQ ID NO: 3350.

The probes having the nucleotide sequence represented by SEQ ID NOS: 42968, 42969, 42970, 42971, 42972, 42973, 42974, 42975, 42976, 42977, 42978 were selected from the ORFs having the nucleotide sequence represented by SEQ ID NO: 3351.

The probes having the nucleotide sequence represented by SEQ ID NOS: 42979, 42980, 42981, 42982, 42983, 42984, 42985, 42986, 42987, 42988, 42989 were selected from the ORFs having the nucleotide sequence represented by SEQ ID NO: 3352.

The probes having the nucleotide sequence represented by SEQ ID NOS: 42990, 42991, 42992, 42993, 42994, 42995, 42996, 42997, 42998, 42999, 43000 were selected from the ORFs having the nucleotide sequence represented by SEQ ID NO: 3353.

The probes having the nucleotide sequence represented by SEQ ID NOS: 43001, 43002, 43003, 43004, 43005, 43006, 43007, 43008, 43009, 43010, 43011 were selected from the ORFs having the nucleotide sequence represented by SEQ ID NO: 3354.

The probes having the nucleotide sequence represented by SEQ ID NOS: 43012, 43013, 43014, 43015, 43016, 43017, 43018, 43019, 43020, 43021, 43022 were selected from the ORFs having the nucleotide sequence represented by SEQ ID NO: 3355.

The probes having the nucleotide sequence represented by SEQ ID NOS: 43023, 43024, 43025, 43026, 43027, 43028, 43029, 43030, 43031, 43032, 43033 were selected from the ORFs having the nucleotide sequence represented by SEQ ID NO: 3356.

The probes having the nucleotide sequence represented by SEQ ID NOS: 43034, 43035, 43036, 43037, 43038, 43039, 43040, 43041, 43042, 43043, 43044 were selected from the ORFs having the nucleotide sequence represented by SEQ ID NO: 3357.

The probes having the nucleotide sequence represented by SEQ ID NOS: 43045, 43046, 43047, 43048, 43049, 43050, 43051, 43052, 43053, 43054, 43055 were selected from the ORFs having the nucleotide sequence represented by SEQ ID NO: 3358.

The probes having the nucleotide sequence represented by SEQ ID NOS: 43056, 43057, 43058, 43059, 43060, 43061, 43062, 43063, 43064, 43065, 43066 were selected from the ORFs having the nucleotide sequence represented by SEQ ID NO: 3359.

The probes having the nucleotide sequence represented by SEQ ID NOS: 43067, 43068, 43069, 43070, 43071, 43072, 43073, 43074, 43075, 43076, 43077 were selected from the ORFs having the nucleotide sequence represented by SEQ ID NO: 3360.

The probes having the nucleotide sequence represented by SEQ ID NOS: 43078, 43079, 43080, 43081, 43082, 43083, 43084, 43085, 43086, 43087, 43088 were selected from the ORFs having the nucleotide sequence represented by SEQ ID NO: 3361.

The probes having the nucleotide sequence represented by SEQ ID NOS: 43089, 43090, 43091, 43092, 43093, 43094, 43095, 43096, 43097, 43098, 43099 were selected from the ORFs having the nucleotide sequence represented by SEQ ID NO: 3362.

The probes having the nucleotide sequence represented by SEQ ID NOS: 43100, 43101, 43102, 43103, 43104, 43105, 43106, 43107, 43108, 43109, 43110 were selected from the ORFs having the nucleotide sequence represented by SEQ ID NO: 3363.

The probes having the nucleotide sequence represented by SEQ ID NOS: 43111, 43112, 43113, 43114, 43115, 43116, 43117, 43118, 43119, 43120, 43121 were selected from the ORFs having the nucleotide sequence represented by SEQ ID NO: 3364.

The probes having the nucleotide sequence represented by SEQ ID NOS: 43122, 43123, 43124, 43125, 43126, 43127, 43128, 43129, 43130, 43131, 43132 were selected from the ORFs having the nucleotide sequence represented by SEQ ID NO: 3365.

The probes having the nucleotide sequence represented by SEQ ID NOS: 43133, 43134, 43135, 43136, 43137, 43138, 43139, 43140, 43141, 43142, 43143 were selected from the ORFs having the nucleotide sequence represented by SEQ ID NO: 3366.

The probes having the nucleotide sequence represented by SEQ ID NOS: 43144, 43145, 43146, 43147, 43148, 43149, 43150, 43151, 43152, 43153, 43154 were selected from the ORFs having the nucleotide sequence represented by SEQ ID NO: 3367.

The probes having the nucleotide sequence represented by SEQ ID NOS: 43155, 43156, 43157, 43158, 43159, 43160, 43161, 43162, 43163, 43164, 43165 were selected from the ORFs having the nucleotide sequence represented by SEQ ID NO: 3368.

The probes having the nucleotide sequence represented by SEQ ID NOS: 43166, 43167, 43168, 43169, 43170, 43171, 43172, 43173, 43174, 43175, 43176 were selected from the ORFs having the nucleotide sequence represented by SEQ ID NO: 3369.

The probes having the nucleotide sequence represented by SEQ ID NOS: 43177, 43178, 43179, 43180, 43181, 43182, 43183, 43184, 43185, 43186, 43187 were selected from the ORFs having the nucleotide sequence represented by SEQ ID NO: 3370.

The probes having the nucleotide sequence represented by SEQ ID NOS: 43188, 43189, 43190, 43191, 43192, 43193, 43194, 43195, 43196, 43197, 43198 were selected from the ORFs having the nucleotide sequence represented by SEQ ID NO: 3371.

The probes having the nucleotide sequence represented by SEQ ID NOS: 43199, 43200, 43201, 43202, 43203, 43204, 43205, 43206, 43207, 43208, 43209 were selected from the ORFs having the nucleotide sequence represented by SEQ ID NO: 3372.

The probes having the nucleotide sequence represented by SEQ ID NOS: 43210, 43211, 43212, 43213, 43214, 43215, 43216, 43217, 43218, 43219, 43220 were selected from the ORFs having the nucleotide sequence represented by SEQ ID NO: 3373.

The probes having the nucleotide sequence represented by SEQ ID NOS: 43221, 43222, 43223, 43224, 43225, 43226, 43227, 43228, 43229, 43230, 43231 were selected from the ORFs having the nucleotide sequence represented by SEQ ID NO: 3374.

The probes having the nucleotide sequence represented by SEQ ID NOS: 43232, 43233, 43234, 43235, 43236, 43237, 43238, 43239, 43240, 43241, 43242 were selected from the ORFs having the nucleotide sequence represented by SEQ ID NO: 3375.

The probes having the nucleotide sequence represented by SEQ ID NOS: 43243, 43244, 43245, 43246, 43247, 43248, 43249, 43250, 43251, 43252, 43253 were selected from the ORFs having the nucleotide sequence represented by SEQ ID NO: 3376.

The probes having the nucleotide sequence represented by SEQ ID NOS: 43254, 43255, 43256, 43257, 43258, 43259, 43260, 43261, 43262, 43263, 43264 were selected from the ORFs having the nucleotide sequence represented by SEQ ID NO: 3377.

The probes having the nucleotide sequence represented by SEQ ID NOS: 43265, 43266, 43267, 43268, 43269, 43270, 43271, 43272, 43273, 43274, 43275 were selected from the ORFs having the nucleotide sequence represented by SEQ ID NO: 3378.

The probes having the nucleotide sequence represented by SEQ ID NOS: 43276, 43277, 43278, 43279, 43280, 43281, 43282, 43283, 43284, 43285, 43286 were selected from the ORFs having the nucleotide sequence represented by SEQ ID NO: 3379.

The probes having the nucleotide sequence represented by SEQ ID NOS: 43287, 43288, 43289, 43290, 43291, 43292, 43293, 43294, 43295, 43296, 43297 were selected from the ORFs having the nucleotide sequence represented by SEQ ID NO: 3380.

The probes having the nucleotide sequence represented by SEQ ID NOS: 43298, 43299, 43300, 43301, 43302, 43303, 43304, 43305, 43306, 43307, 43308 were selected from the ORFs having the nucleotide sequence represented by SEQ ID NO: 3381.

The probes having the nucleotide sequence represented by SEQ ID NOS: 43309, 43310, 43311, 43312, 43313, 43314, 43315, 43316, 43317, 43318, 43319 were selected from the ORFs having the nucleotide sequence represented by SEQ ID NO: 3382.

The probes having the nucleotide sequence represented by SEQ ID NOS: 43320, 43321, 43322, 43323, 43324, 43325, 43326, 43327, 43328, 43329, 43330 were selected from the ORFs having the nucleotide sequence represented by SEQ ID NO: 3383.

The probes having the nucleotide sequence represented by SEQ ID NOS: 43331, 43332, 43333, 43334, 43335, 43336, 43337, 43338, 43339, 43340, 43341 were selected from the ORFs having the nucleotide sequence represented by SEQ ID NO: 3384.

The probes having the nucleotide sequence represented by SEQ ID NOS: 43342, 43343, 43344, 43345, 43346, 43347, 43348, 43349, 43350, 43351, 43352 were selected from the ORFs having the nucleotide sequence represented by SEQ ID NO: 3385.

The probes having the nucleotide sequence represented by SEQ ID NOS: 43353, 43354, 43355, 43356, 43357, 43358, 43359, 43360, 43361, 43362, 43363 were selected from the ORFs having the nucleotide sequence represented by SEQ ID NO: 3386.

The probes having the nucleotide sequence represented by SEQ ID NOS: 43364, 43365, 43366, 43367, 43368, 43369, 43370, 43371, 43372, 43373, 43374 were selected from the ORFs having the nucleotide sequence represented by SEQ ID NO: 3387.

The probes having the nucleotide sequence represented by SEQ ID NOS: 43375, 43376, 43377, 43378, 43379, 43380, 43381, 43382, 43383, 43384, 43385 were selected from the ORFs having the nucleotide sequence represented by SEQ ID NO: 3388.

The probes having the nucleotide sequence represented by SEQ ID NOS: 43386, 43387, 43388, 43389, 43390, 43391, 43392, 43393, 43394, 43395, 43396 were selected from the ORFs having the nucleotide sequence represented by SEQ ID NO: 3389.

The probes having the nucleotide sequence represented by SEQ ID NOS: 43397, 43398, 43399, 43400, 43401, 43402, 43403, 43404, 43405, 43406, 43407 were selected from the ORFs having the nucleotide sequence represented by SEQ ID NO: 3390.

The probes having the nucleotide sequence represented by SEQ ID NOS: 43408, 43409, 43410, 43411, 43412, 43413, 43414, 43415, 43416, 43417, 43418 were selected from the ORFs having the nucleotide sequence represented by SEQ ID NO: 3391.

The probes having the nucleotide sequence represented by SEQ ID NOS: 43419, 43420, 43421, 43422, 43423, 43424, 43425, 43426, 43427, 43428, 43429 were selected from the ORFs having the nucleotide sequence represented by SEQ ID NO: 3392.

The probes having the nucleotide sequence represented by SEQ ID NOS: 43430, 43431, 43432, 43433, 43434, 43435, 43436, 43437, 43438, 43439, 43440 were selected from the ORFs having the nucleotide sequence represented by SEQ ID NO: 3393.

The probes having the nucleotide sequence represented by SEQ ID NOS: 43441, 43442, 43443, 43444, 43445, 43446, 43447, 43448, 43449, 43450, 43451 were selected from the ORFs having the nucleotide sequence represented by SEQ ID NO: 3394.

The probes having the nucleotide sequence represented by SEQ ID NOS: 43452, 43453, 43454, 43455, 43456, 43457, 43458, 43459, 43460, 43461, 43462 were selected from the ORFs having the nucleotide sequence represented by SEQ ID NO: 3395.

The probes having the nucleotide sequence represented by SEQ ID NOS: 43463, 43464, 43465, 43466, 43467, 43468, 43469, 43470, 43471, 43472, 43473 were selected from the ORFs having the nucleotide sequence represented by SEQ ID NO: 3396.

The probes having the nucleotide sequence represented by SEQ ID NOS: 43474, 43475, 43476, 43477, 43478, 43479, 43480, 43481, 43482, 43483, 43484 were selected from the ORFs having the nucleotide sequence represented by SEQ ID NO: 3397.

The probes having the nucleotide sequence represented by SEQ ID NOS: 43485, 43486, 43487, 43488, 43489, 43490, 43491, 43492, 43493, 43494, 43495 were selected from the ORFs having the nucleotide sequence represented by SEQ ID NO: 3398.

The probes having the nucleotide sequence represented by SEQ ID NOS: 43496, 43497, 43498, 43499, 43500, 43501, 43502, 43503, 43504, 43505, 43506 were selected from the ORFs having the nucleotide sequence represented by SEQ ID NO: 3399.

The probes having the nucleotide sequence represented by SEQ ID NOS: 43507, 43508, 43509, 43510, 43511, 43512, 43513, 43514, 43515, 43516, 43517 were selected from the ORFs having the nucleotide sequence represented by SEQ ID NO: 3400.

The probes having the nucleotide sequence represented by SEQ ID NOS: 43518, 43519, 43520, 43521, 43522, 43523, 43524, 43525, 43526, 43527, 43528 were selected from the ORFs having the nucleotide sequence represented by SEQ ID NO: 3401.

The probes having the nucleotide sequence represented by SEQ ID NOS: 43529, 43530, 43531, 43532, 43533, 43534, 43535, 43536, 43537, 43538, 43539, 43540, 43541, 43542, 43543, 43544, 43545, 43546, 43547, 43548, 43549, 43550 were selected from the ORFs having the nucleotide sequence represented by SEQ ID NO: 3402.

The probes having the nucleotide sequence represented by SEQ ID NOS: 43551, 43552, 43553, 43554, 43555, 43556, 43557, 43558, 43559, 43560, 43561 were selected from the ORFs having the nucleotide sequence represented by SEQ ID NO: 3403.

The probes having the nucleotide sequence represented by SEQ ID NOS: 43562, 43563, 43564, 43565, 43566, 43567, 43568, 43569, 43570, 43571, 43572 were selected from the ORFs having the nucleotide sequence represented by SEQ ID NO: 3404.

The probes having the nucleotide sequence represented by SEQ ID NOS: 43573, 43574, 43575, 43576, 43577, 43578, 43579, 43580, 43581, 43582, 43583 were selected from the ORFs having the nucleotide sequence represented by SEQ ID NO: 3405.

The probes having the nucleotide sequence represented by SEQ ID NOS: 43584, 43585, 43586, 43587, 43588, 43589, 43590, 43591, 43592, 43593, 43594 were selected from the ORFs having the nucleotide sequence represented by SEQ ID NO: 3406.

The probes having the nucleotide sequence represented by SEQ ID NOS: 43595, 43596, 43597, 43598, 43599, 43600, 43601, 43602, 43603, 43604, 43605 were selected from the ORFs having the nucleotide sequence represented by SEQ ID NO: 3407.

The probes having the nucleotide sequence represented by SEQ ID NOS: 43606, 43607, 43608, 43609, 43610, 43611, 43612, 43613, 43614, 43615, 43616 were selected from the ORFs having the nucleotide sequence represented by SEQ ID NO: 3408.

The probes having the nucleotide sequence represented by SEQ ID NOS: 43617, 43618, 43619, 43620, 43621, 43622, 43623, 43624, 43625, 43626, 43627 were selected from the ORFs having the nucleotide sequence represented by SEQ ID NO: 3409.

The probes having the nucleotide sequence represented by SEQ ID NOS: 43628, 43629, 43630, 43631, 43632, 43633, 43634, 43635, 43636, 43637, 43638 were selected from the ORFs having the nucleotide sequence represented by SEQ ID NO: 3410.

The probes having the nucleotide sequence represented by SEQ ID NOS: 43639, 43640, 43641, 43642, 43643, 43644, 43645, 43646, 43647, 43648, 43649 were selected from the ORFs having the nucleotide sequence represented by SEQ ID NO: 3411.

The probes having the nucleotide sequence represented by SEQ ID NOS: 43650, 43651, 43652, 43653, 43654, 43655, 43656, 43657, 43658, 43659, 43660, 43661, 43662, 43663, 43664, 43665, 43666, 43667, 43668, 43669, 43670, 43671 were selected from the ORFs having the nucleotide sequence represented by SEQ ID NO: 3412.

The probes having the nucleotide sequence represented by SEQ ID NOS: 43672, 43673, 43674, 43675, 43676, 43677, 43678, 43679, 43680, 43681, 43682 were selected from the ORFs having the nucleotide sequence represented by SEQ ID NO: 3413.

The probes having the nucleotide sequence represented by SEQ ID NOS: 43683, 43684, 43685, 43686, 43687, 43688, 43689, 43690, 43691, 43692, 43693 were selected from the ORFs having the nucleotide sequence represented by SEQ ID NO: 3414.

The probes having the nucleotide sequence represented by SEQ ID NOS: 43694, 43695, 43696, 43697, 43698, 43699, 43700, 43701, 43702, 43703, 43704 were selected from the ORFs having the nucleotide sequence represented by SEQ ID NO: 3415.

The probes having the nucleotide sequence represented by SEQ ID NOS: 43705, 43706, 43707, 43708, 43709, 43710, 43711, 43712, 43713, 43714, 43715 were selected from the ORFs having the nucleotide sequence represented by SEQ ID NO: 3416.

The probes having the nucleotide sequence represented by SEQ ID NOS: 43716, 43717, 43718, 43719, 43720, 43721, 43722, 43723, 43724, 43725, 43726 were selected from the ORFs having the nucleotide sequence represented by SEQ ID NO: 3417.

The probes having the nucleotide sequence represented by SEQ ID NOS: 43727, 43728, 43729, 43730, 43731, 43732, 43733, 43734, 43735, 43736, 43737 were selected from the ORFs having the nucleotide sequence represented by SEQ ID NO: 3418.

The probes having the nucleotide sequence represented by SEQ ID NOS: 43738, 43739, 43740, 43741, 43742, 43743, 43744, 43745, 43746, 43747, 43748 were selected from the ORFs having the nucleotide sequence represented by SEQ ID NO: 3419.

The probes having the nucleotide sequence represented by SEQ ID NOS: 43749, 43750, 43751, 43752, 43753, 43754, 43755, 43756, 43757, 43758, 43759 were selected from the ORFs having the nucleotide sequence represented by SEQ ID NO: 3420.

The probes having the nucleotide sequence represented by SEQ ID NOS: 43760, 43761, 43762, 43763, 43764, 43765, 43766, 43767, 43768, 43769, 43770 were selected from the ORFs having the nucleotide sequence represented by SEQ ID NO: 3421.

The probes having the nucleotide sequence represented by SEQ ID NOS: 43771, 43772, 43773, 43774, 43775, 43776, 43777, 43778, 43779, 43780, 43781 were selected from the ORFs having the nucleotide sequence represented by SEQ ID NO: 3422.

The probes having the nucleotide sequence represented by SEQ ID NOS: 43782, 43783, 43784, 43785, 43786, 43787, 43788, 43789, 43790, 43791, 43792 were selected from the ORFs having the nucleotide sequence represented by SEQ ID NO: 3423.

The probes having the nucleotide sequence represented by SEQ ID NOS: 43793, 43794, 43795, 43796, 43797, 43798, 43799, 43800, 43801, 43802, 43803, 43804, 43805, 43806, 43807, 43808, 43809, 43810, 43811, 43812, 43813, 43814 were selected from the ORFs having the nucleotide sequence represented by SEQ ID NO: 3424.

The probes having the nucleotide sequence represented by SEQ ID NOS: 43815, 43816, 43817, 43818, 43819, 43820, 43821, 43822, 43823, 43824, 43825 were selected from the ORFs having the nucleotide sequence represented by SEQ ID NO: 3425.

The probes having the nucleotide sequence represented by SEQ ID NOS: 43826, 43827, 43828, 43829, 43830, 43831, 43832, 43833, 43834, 43835, 43836 were selected from the ORFs having the nucleotide sequence represented by SEQ ID NO: 3426.

The probes having the nucleotide sequence represented by SEQ ID NOS: 43837, 43838, 43839, 43840, 43841, 43842, 43843, 43844, 43845, 43846, 43847 were selected from the ORFs having the nucleotide sequence represented by SEQ ID NO: 3427.

The probes having the nucleotide sequence represented by SEQ ID NOS: 43848, 43849, 43850, 43851, 43852, 43853, 43854, 43855, 43856, 43857, 43858 were selected from the ORFs having the nucleotide sequence represented by SEQ ID NO: 3428.

The probes having the nucleotide sequence represented by SEQ ID NOS: 43859, 43860, 43861, 43862, 43863, 43864, 43865, 43866, 43867, 43868, 43869 were selected from the ORFs having the nucleotide sequence represented by SEQ ID NO: 3429.

The probes having the nucleotide sequence represented by SEQ ID NOS: 43870, 43871, 43872, 43873, 43874, 43875, 43876, 43877, 43878, 43879, 43880 were selected from the ORFs having the nucleotide sequence represented by SEQ ID NO: 3430.

The probes having the nucleotide sequence represented by SEQ ID NOS: 43881, 43882, 43883, 43884, 43885, 43886, 43887, 43888, 43889, 43890, 43891 were selected from the ORFs having the nucleotide sequence represented by SEQ ID NO: 3431.

The probes having the nucleotide sequence represented by SEQ ID NOS: 43892, 43893, 43894, 43895, 43896, 43897, 43898, 43899, 43900, 43901, 43902 were selected from the ORFs having the nucleotide sequence represented by SEQ ID NO: 3432.

The probes having the nucleotide sequence represented by SEQ ID NOS: 43903, 43904, 43905, 43906, 43907, 43908, 43909, 43910, 43911, 43912, 43913 were selected from the ORFs having the nucleotide sequence represented by SEQ ID NO: 3433.

The probes having the nucleotide sequence represented by SEQ ID NOS: 43914, 43915, 43916, 43917, 43918, 43919, 43920, 43921, 43922, 43923, 43924 were selected from the ORFs having the nucleotide sequence represented by SEQ ID NO: 3434.

The probes having the nucleotide sequence represented by SEQ ID NOS: 43925, 43926, 43927, 43928, 43929, 43930, 43931, 43932, 43933, 43934, 43935 were selected from the ORFs having the nucleotide sequence represented by SEQ ID NO: 3435.

The probes having the nucleotide sequence represented by SEQ ID NOS: 43936, 43937, 43938, 43939, 43940, 43941, 43942, 43943, 43944, 43945, 43946 were selected from the ORFs having the nucleotide sequence represented by SEQ ID NO: 3436.

The probes having the nucleotide sequence represented by SEQ ID NOS: 43947, 43948, 43949, 43950, 43951, 43952, 43953, 43954, 43955, 43956, 43957 were selected from the ORFs having the nucleotide sequence represented by SEQ ID NO: 3437.

The probes having the nucleotide sequence represented by SEQ ID NOS: 43958, 43959, 43960, 43961, 43962, 43963, 43964, 43965, 43966, 43967, 43968 were selected from the ORFs having the nucleotide sequence represented by SEQ ID NO: 3438.

The probes having the nucleotide sequence represented by SEQ ID NOS: 43969, 43970, 43971, 43972, 43973, 43974, 43975, 43976, 43977, 43978, 43979 were selected from the ORFs having the nucleotide sequence represented by SEQ ID NO: 3439.

The probes having the nucleotide sequence represented by SEQ ID NOS: 43980, 43981, 43982, 43983, 43984, 43985, 43986, 43987, 43988, 43989, 43990 were selected from the ORFs having the nucleotide sequence represented by SEQ ID NO: 3440.

The probes having the nucleotide sequence represented by SEQ ID NOS: 43991, 43992, 43993, 43994, 43995, 43996, 43997, 43998, 43999, 44000, 44001 were selected from the ORFs having the nucleotide sequence represented by SEQ ID NO: 3441.

The probes having the nucleotide sequence represented by SEQ ID NOS: 44002, 44003, 44004, 44005, 44006, 44007, 44008, 44009, 44010, 44011, 44012.were selected from the ORFs having the nucleotide sequence represented by SEQ ID NO: 3442.

The probes having the nucleotide sequence represented by SEQ ID NOS: 44013, 44014, 44015, 44016, 44017, 44018, 44019, 44020, 44021, 44022, 44023 were selected from the ORFs having the nucleotide sequence represented by SEQ ID NO: 3443.

The probes having the nucleotide sequence represented by SEQ ID NOS: 44024, 44025, 44026, 44027, 44028, 44029, 44030, 44031, 44032, 44033, 44034 were selected from the ORFs having the nucleotide sequence represented by SEQ ID NO: 3444.

The probes having the nucleotide sequence represented by SEQ ID NOS: 44035, 44036, 44037, 44038, 44039, 44040, 44041, 44042, 44043, 44044, 44045 were selected from the ORFs having the nucleotide sequence represented by SEQ ID NO: 3445.

The probes having the nucleotide sequence represented by SEQ ID NOS: 44046, 44047, 44048, 44049, 44050, 44051, 44052, 44053, 44054, 44055, 44056 were selected from the ORFs having the nucleotide sequence represented by SEQ ID NO: 3446.

The probes having the nucleotide sequence represented by SEQ ID NOS: 44057, 44058, 44059, 44060, 44061, 44062, 44063, 44064, 44065, 44066, 44067 were selected from the ORFs having the nucleotide sequence represented by SEQ ID NO: 3447.

The probes having the nucleotide sequence represented by SEQ ID NOS: 44068, 44069, 44070, 44071, 44072, 44073, 44074, 44075, 44076, 44077, 44078 were selected from the ORFs having the nucleotide sequence represented by SEQ ID NO: 3448.

The probes having the nucleotide sequence represented by SEQ ID NOS: 44079, 44080, 44081, 44082, 44083, 44084, 44085, 44086, 44087, 44088, 44089 were selected from the ORFs having the nucleotide sequence represented by SEQ ID NO: 3449.

The probes having the nucleotide sequence represented by SEQ ID NOS: 44090, 44091, 44092, 44093, 44094, 44095, 44096, 44097, 44098, 44099, 44100 were selected from the ORFs having the nucleotide sequence represented by SEQ ID NO: 3450.

The probes having the nucleotide sequence represented by SEQ ID NOS: 44101, 44102, 44103, 44104, 44105, 44106, 44107, 44108, 44109, 44110, 44111 were selected from the ORFs having the nucleotide sequence represented by SEQ ID NO: 3451.

The probes having the nucleotide sequence represented by SEQ ID NOS: 44112, 44113, 44114, 44115, 44116, 44117, 44118, 44119, 44120, 44121, 44122 were selected from the ORFs having the nucleotide sequence represented by SEQ ID NO: 3452.

The probes having the nucleotide sequence represented by SEQ ID NOS: 44123, 44124, 44125, 44126, 44127, 44128, 44129, 44130, 44131, 44132, 44133 were selected from the ORFs having the nucleotide sequence represented by SEQ ID NO: 3453.

The probes having the nucleotide sequence represented by SEQ ID NOS: 44134, 44135, 44136, 44137, 44138, 44139, 44140, 44141, 44142, 44143, 44144 were selected from the ORFs having the nucleotide sequence represented by SEQ ID NO: 3454.

The probes having the nucleotide sequence represented by SEQ ID NOS: 44145, 44146, 44147, 44148, 44149, 44150, 44151, 44152, 44153, 44154, 44155 were selected from the ORFs having the nucleotide sequence represented by SEQ ID NO: 3455.

The probes having the nucleotide sequence represented by SEQ ID NOS: 44156, 44157, 44158, 44159, 44160, 44161, 44162, 44163, 44164, 44165, 44166 were selected from the ORFs having the nucleotide sequence represented by SEQ ID NO: 3456.

The probes having the nucleotide sequence represented by SEQ ID NOS: 44167, 44168, 44169, 44170, 44171, 44172, 44173, 44174, 44175, 44176, 44177 were selected from the ORFs having the nucleotide sequence represented by SEQ ID NO: 3457.

The probes having the nucleotide sequence represented by SEQ ID NOS: 44178, 44179, 44180, 44181, 44182, 44183, 44184, 44185, 44186, 44187, 44188 were selected from the ORFs having the nucleotide sequence represented by SEQ ID NO: 3458.

The probes having the nucleotide sequence represented by SEQ ID NOS: 44189, 44190, 44191, 44192, 44193, 44194, 44195, 44196, 44197, 44198, 44199 were selected from the ORFs having the nucleotide sequence represented by SEQ ID NO: 3459.

The probes having the nucleotide sequence represented by SEQ ID NOS: 44200, 44201, 44202, 44203, 44204, 44205, 44206, 44207, 44208, 44209, 44210 were selected from the ORFs having the nucleotide sequence represented by SEQ ID NO: 3460.

The probes having the nucleotide sequence represented by SEQ ID NOS: 44211, 44212, 44213, 44214, 44215, 44216, 44217, 44218, 44219, 44220, 44221 were selected from the ORFs having the nucleotide sequence represented by SEQ ID NO: 3461.

The probes having the nucleotide sequence represented by SEQ ID NOS: 44222, 44223, 44224, 44225, 44226, 44227, 44228, 44229, 44230, 44231, 44232 were selected from the ORFs having the nucleotide sequence represented by SEQ ID NO: 3462.

The probes having the nucleotide sequence represented by SEQ ID NOS: 44233, 44234, 44235, 44236, 44237, 44238, 44239, 44240, 44241, 44242, 44243 were selected from the ORFs having the nucleotide sequence represented by SEQ ID NO: 3463.

The probes having the nucleotide sequence represented by SEQ ID NOS: 44244, 44245, 44246, 44247, 44248, 44249, 44250, 44251, 44252, 44253, 44254 were selected from the ORFs having the nucleotide sequence represented by SEQ ID NO: 3464.

The probes having the nucleotide sequence represented by SEQ ID NOS: 44255, 44256, 44257, 44258, 44259, 44260, 44261, 44262, 44263, 44264, 44265 were selected from the ORFs having the nucleotide sequence represented by SEQ ID NO: 3465.

The probes having the nucleotide sequence represented by SEQ ID NOS: 44266, 44267, 44268, 44269, 44270, 44271, 44272, 44273, 44274, 44275, 44276 were selected from the ORFs having the nucleotide sequence represented by SEQ ID NO: 3466.

The probes having the nucleotide sequence represented by SEQ ID NOS: 44277, 44278, 44279, 44280, 44281, 44282, 44283, 44284, 44285, 44286, 44287 were selected from the ORFs having the nucleotide sequence represented by SEQ ID NO: 3467.

The probes having the nucleotide sequence represented by SEQ ID NOS: 44288, 44289, 44290, 44291, 44292, 44293, 44294, 44295, 44296, 44297, 44298 were selected from the ORFs having the nucleotide sequence represented by SEQ ID NO: 3468.

The probes having the nucleotide sequence represented by SEQ ID NOS: 44299, 44300, 44301, 44302, 44303, 44304, 44305, 44306, 44307, 44308, 44309 were selected from the ORFs having the nucleotide sequence represented by SEQ ID NO: 3469.

The probes having the nucleotide sequence represented by SEQ ID NOS: 44310, 44311, 44312, 44313, 44314, 44315, 44316, 44317, 44318, 44319, 44320 were selected from the ORFs having the nucleotide sequence represented by SEQ ID NO: 3470.

The probes having the nucleotide sequence represented by SEQ ID NOS: 44321, 44322, 44323, 44324, 44325, 44326, 44327, 44328, 44329, 44330, 44331 were selected from the ORFs having the nucleotide sequence represented by SEQ ID NO: 3471.

The probes having the nucleotide sequence represented by SEQ ID NOS: 44332, 44333, 44334, 44335, 44336, 44337, 44338, 44339, 44340, 44341, 44342 were selected from the ORFs having the nucleotide sequence represented by SEQ ID NO: 3472.

The probes having the nucleotide sequence represented by SEQ ID NOS: 44343, 44344, 44345, 44346, 44347, 44348, 44349, 44350, 44351, 44352, 44353 were selected from the ORFs having the nucleotide sequence represented by SEQ ID NO: 3473.

The probes having the nucleotide sequence represented by SEQ ID NOS: 44354, 44355, 44356, 44357, 44358, 44359, 44360, 44361, 44362, 44363, 44364 were selected from the ORFs having the nucleotide sequence represented by SEQ ID NO: 3474.

The probes having the nucleotide sequence represented by SEQ ID NOS: 44365, 44366, 44367, 44368, 44369, 44370, 44371, 44372, 44373, 44374, 44375 were selected from the ORFs having the nucleotide sequence represented by SEQ ID NO: 3475.

The probes having the nucleotide sequence represented by SEQ ID NOS: 44376, 44377, 44378, 44379, 44380, 44381, 44382, 44383, 44384, 44385, 44386 were selected from the ORFs having the nucleotide sequence represented by SEQ ID NO: 3476.

The probes having the nucleotide sequence represented by SEQ ID NOS: 44387, 44388, 44389, 44390, 44391, 44392, 44393, 44394, 44395, 44396, 44397 were selected from the ORFs having the nucleotide sequence represented by SEQ ID NO: 3477.

The probes having the nucleotide sequence represented by SEQ ID NOS: 44398, 44399, 44400, 44401, 44402, 44403, 44404, 44405, 44406, 44407, 44408 were selected from the ORFs having the nucleotide sequence represented by SEQ ID NO: 3478.

The probes having the nucleotide sequence represented by SEQ ID NOS: 44409, 44410, 44411, 44412, 44413, 44414, 44415, 44416, 44417, 44418, 44419 were selected from the ORFs having the nucleotide sequence represented by SEQ ID NO: 3479.

The probes having the nucleotide sequence represented by SEQ ID NOS: 44420, 44421, 44422, 44423, 44424, 44425, 44426, 44427, 44428, 44429, 44430 were selected from the ORFs having the nucleotide sequence represented by SEQ ID NO: 3480.

The probes having the nucleotide sequence represented by SEQ ID NOS: 44431, 44432, 44433, 44434, 44435, 44436, 44437, 44438, 44439, 44440, 44441 were selected from the ORFs having the nucleotide sequence represented by SEQ ID NO: 3481.

The probes having the nucleotide sequence represented by SEQ ID NOS: 44442, 44443, 44444, 44445, 44446, 44447, 44448, 44449, 44450, 44451, 44452 were selected from the ORFs having the nucleotide sequence represented by SEQ ID NO: 3482.

The probes having the nucleotide sequence represented by SEQ ID NOS: 44453, 44454, 44455, 44456, 44457, 44458, 44459, 44460, 44461, 44462, 44463 were selected from the ORFs having the nucleotide sequence represented by SEQ ID NO: 3483.

The probes having the nucleotide sequence represented by SEQ ID NOS: 44464, 44465, 44466, 44467, 44468, 44469, 44470, 44471, 44472, 44473, 44474 were selected from the ORFs having the nucleotide sequence represented by SEQ ID NO: 3484.

The probes having the nucleotide sequence represented by SEQ ID NOS: 44475, 44476, 44477, 44478, 44479, 44480, 44481, 44482, 44483, 44484, 44485 were selected from the ORFs having the nucleotide sequence represented by SEQ ID NO: 3485.

The probes having the nucleotide sequence represented by SEQ ID NOS: 44486, 44487, 44488, 44489, 44490, 44491, 44492, 44493, 44494, 44495, 44496 were selected from the ORFs having the nucleotide sequence represented by SEQ ID NO: 3486.

The probes having the nucleotide sequence represented by SEQ ID NOS: 44497, 44498, 44499, 44500, 44501, 44502, 44503, 44504, 44505, 44506, 44507 were selected from the ORFs having the nucleotide sequence represented by SEQ ID NO: 3487.

The probes having the nucleotide sequence represented by SEQ ID NOS: 44508, 44509, 44510, 44511, 44512, 44513, 44514, 44515, 44516, 44517, 44518 were selected from the ORFs having the nucleotide sequence represented by SEQ ID NO: 3488.

The probes having the nucleotide sequence represented by SEQ ID NOS: 44519, 44520, 44521, 44522, 44523, 44524, 44525, 44526, 44527, 44528, 44529 were selected from the ORFs having the nucleotide sequence represented by SEQ ID NO: 3489.

The probes having the nucleotide sequence represented by SEQ ID NOS: 44530, 44531, 44532, 44533, 44534, 44535, 44536, 44537, 44538, 44539, 44540 were selected from the ORFs having the nucleotide sequence represented by SEQ ID NO: 3490.

The probes having the nucleotide sequence represented by SEQ ID NOS: 44541, 44542, 44543, 44544, 44545, 44546, 44547, 44548, 44549, 44550, 44551 were selected from the ORFs having the nucleotide sequence represented by SEQ ID NO: 3491.

The probes having the nucleotide sequence represented by SEQ ID NOS: 44552, 44553, 44554, 44555, 44556, 44557, 44558, 44559, 44560, 44561, 44562 were selected from the ORFs having the nucleotide sequence represented by SEQ ID NO: 3492.

The probes having the nucleotide sequence represented by SEQ ID NOS: 44563, 44564, 44565, 44566, 44567, 44568, 44569, 44570, 44571, 44572, 44573 were selected from the ORFs having the nucleotide sequence represented by SEQ ID NO: 3493.

The probes having the nucleotide sequence represented by SEQ ID NOS: 44574, 44575, 44576, 44577, 44578, 44579, 44580, 44581, 44582, 44583, 44584 were selected from the ORFs having the nucleotide sequence represented by SEQ ID NO: 3494.

The probes having the nucleotide sequence represented by SEQ ID NOS: 44585, 44586, 44587, 44588, 44589, 44590, 44591, 44592, 44593, 44594, 44595 were selected from the ORFs having the nucleotide sequence represented by SEQ ID NO: 3495.

The probes having the nucleotide sequence represented by SEQ ID NOS: 44596, 44597, 44598, 44599, 44600, 44601, 44602, 44603, 44604, 44605, 44606 were selected from the ORFs having the nucleotide sequence represented by SEQ ID NO: 3496.

The probes having the nucleotide sequence represented by SEQ ID NOS: 44607, 44608, 44609, 44610, 44611, 44612, 44613, 44614, 44615, 44616, 44617 were selected from the ORFs having the nucleotide sequence represented by SEQ ID NO: 3497.

The probes having the nucleotide sequence represented by SEQ ID NOS: 44618, 44619, 44620, 44621, 44622, 44623, 44624, 44625, 44626, 44627, 44628 were selected from the ORFs having the nucleotide sequence represented by SEQ ID NO: 3498.

The probes having the nucleotide sequence represented by SEQ ID NOS: 44629, 44630, 44631, 44632, 44633, 44634, 44635, 44636, 44637, 44638, 44639 were selected from the ORFs having the nucleotide sequence represented by SEQ ID NO: 3499.

The probes having the nucleotide sequence represented by SEQ ID NOS: 44640, 44641, 44642, 44643, 44644, 44645, 44646, 44647, 44648, 44649, 44650 were selected from the ORFs having the nucleotide sequence represented by SEQ ID NO: 3500.

The probes having the nucleotide sequence represented by SEQ ID NOS: 44651, 44652, 44653, 44654, 44655, 44656, 44657, 44658, 44659, 44660, 44661, 44662, 44663, 44664, 44665, 44666, 44667, 44668, 44669, 44670, 44671, 44672 were selected from the ORFs having the nucleotide sequence represented by SEQ ID NO: 3501.

The probes having the nucleotide sequence represented by SEQ ID NOS: 44673, 44674, 44675, 44676, 44677, 44678, 44679, 44680, 44681, 44682, 44683 were selected from the ORFs having the nucleotide sequence represented by SEQ ID NO: 3502.

The probes having the nucleotide sequence represented by SEQ ID NOS: 44684, 44685, 44686, 44687, 44688, 44689, 44690, 44691, 44692, 44693, 44694 were selected from the ORFs having the nucleotide sequence represented by SEQ ID NO: 3503.

The probes having the nucleotide sequence represented by SEQ ID NOS: 44695, 44696, 44697, 44698, 44699, 44700, 44701, 44702, 44703, 44704, 44705 were selected from the ORFs having the nucleotide sequence represented by SEQ ID NO: 3504.

The probes having the nucleotide sequence represented by SEQ ID NOS: 44706, 44707, 44708, 44709, 44710, 44711, 44712, 44713, 44714, 44715, 44716 were selected from the ORFs having the nucleotide sequence represented by SEQ ID NO: 3505.

The probes having the nucleotide sequence represented by SEQ ID NOS: 44717, 44718, 44719, 44720, 44721, 44722, 44723, 44724, 44725, 44726, 44727 were selected from the ORFs having the nucleotide sequence represented by SEQ ID NO: 3506.

The probes having the nucleotide sequence represented by SEQ ID NOS: 44728, 44729, 44730, 44731, 44732, 44733, 44734, 44735, 44736, 44737, 44738 were selected from the ORFs having the nucleotide sequence represented by SEQ ID NO: 3507.

The probes having the nucleotide sequence represented by SEQ ID NOS: 44739, 44740, 44741, 44742, 44743, 44744, 44745, 44746, 44747, 44748, 44749 were selected from the ORFs having the nucleotide sequence represented by SEQ ID NO: 3508.

The probes having the nucleotide sequence represented by SEQ ID NOS: 44750, 44751, 44752, 44753, 44754, 44755, 44756, 44757, 44758, 44759, 44760 were selected from the ORFs having the nucleotide sequence represented by SEQ ID NO: 3509.

The probes having the nucleotide sequence represented by SEQ ID NOS: 44761, 44762, 44763, 44764, 44765, 44766, 44767, 44768, 44769, 44770, 44771 were selected from the ORFs having the nucleotide sequence represented by SEQ ID NO: 3510.

The probes having the nucleotide sequence represented by SEQ ID NOS: 44772, 44773, 44774, 44775, 44776, 44777, 44778, 44779, 44780, 44781, 44782 were selected from the ORFs having the nucleotide sequence represented by SEQ ID NO: 3511.

The probes having the nucleotide sequence represented by SEQ ID NOS: 44783, 44784, 44785, 44786, 44787, 44788, 44789, 44790, 44791, 44792, 44793 were selected from the ORFs having the nucleotide sequence represented by SEQ ID NO: 3512.

The probes having the nucleotide sequence represented by SEQ ID NOS: 44794, 44795, 44796, 44797, 44798, 44799, 44800, 44801, 44802, 44803, 44804 were selected from the ORFs having the nucleotide sequence represented by SEQ ID NO: 3513.

The probes having the nucleotide sequence represented by SEQ ID NOS: 44805, 44806, 44807, 44808, 44809, 44810, 44811, 44812, 44813, 44814, 44815 were selected from the ORFs having the nucleotide sequence represented by SEQ ID NO: 3514.

The probes having the nucleotide sequence represented by SEQ ID NOS: 44816, 44817, 44818, 44819, 44820, 44821, 44822, 44823, 44824, 44825, 44826 were selected from the ORFs having the nucleotide sequence represented by SEQ ID NO: 3515.

The probes having the nucleotide sequence represented by SEQ ID NOS: 44827, 44828, 44829, 44830, 44831, 44832, 44833, 44834, 44835, 44836, 44837 were selected from the ORFs having the nucleotide sequence represented by SEQ ID NO: 3516.

The probes having the nucleotide sequence represented by SEQ ID NOS: 44838, 44839, 44840, 44841, 44842, 44843, 44844, 44845, 44846, 44847, 44848 were selected from the ORFs having the nucleotide sequence represented by SEQ ID NO: 3517.

The probes having the nucleotide sequence represented by SEQ ID NOS: 44849, 44850, 44851, 44852, 44853, 44854, 44855, 44856, 44857, 44858, 44859 were selected from the ORFs having the nucleotide sequence represented by SEQ ID NO: 3518.

The probes having the nucleotide sequence represented by SEQ ID NOS: 44860, 44861, 44862, 44863, 44864, 44865, 44866, 44867, 44868, 44869, 44870 were selected from the ORFs having the nucleotide sequence represented by SEQ ID NO: 3519.

The probes having the nucleotide sequence represented by SEQ ID NOS: 44871, 44872, 44873, 44874, 44875, 44876, 44877, 44878, 44879, 44880, 44881 were selected from the ORFs having the nucleotide sequence represented by SEQ ID NO: 3520.

The probes having the nucleotide sequence represented by SEQ ID NOS: 44882, 44883, 44884, 44885, 44886, 44887, 44888, 44889, 44890, 44891, 44892 were selected from the ORFs having the nucleotide sequence represented by SEQ ID NO: 3521.

The probes having the nucleotide sequence represented by SEQ ID NOS: 44893, 44894, 44895, 44896, 44897, 44898, 44899, 44900, 44901, 44902, 44903 were selected from the ORFs having the nucleotide sequence represented by SEQ ID NO: 3522.

The probes having the nucleotide sequence represented by SEQ ID NOS: 44904, 44905, 44906, 44907, 44908, 44909, 44910, 44911, 44912, 44913, 44914 were selected from the ORFs having the nucleotide sequence represented by SEQ ID NO: 3523.

The probes having the nucleotide sequence represented by SEQ ID NOS: 44915, 44916, 44917, 44918, 44919, 44920, 44921, 44922, 44923, 44924, 44925 were selected from the ORFs having the nucleotide sequence represented by SEQ ID NO: 3524.

The probes having the nucleotide sequence represented by SEQ ID NOS: 44926, 44927, 44928, 44929, 44930, 44931, 44932, 44933, 44934, 44935, 44936 were selected from the ORFs having the nucleotide sequence represented by SEQ ID NO: 3525.

The probes having the nucleotide sequence represented by SEQ ID NOS: 44937, 44938, 44939, 44940, 44941, 44942, 44943, 44944, 44945, 44946, 44947 were selected from the ORFs having the nucleotide sequence represented by SEQ ID NO: 3526.

The probes having the nucleotide sequence represented by SEQ ID NOS: 44948, 44949, 44950, 44951, 44952, 44953, 44954, 44955, 44956, 44957, 44958 were selected from the ORFs having the nucleotide sequence represented by SEQ ID NO: 3527.

The probes having the nucleotide sequence represented by SEQ ID NOS: 44959, 44960, 44961, 44962, 44963, 44964, 44965, 44966, 44967, 44968, 44969 were selected from the ORFs having the nucleotide sequence represented by SEQ ID NO: 3528.

The probes having the nucleotide sequence represented by SEQ ID NOS: 44970, 44971, 44972, 44973, 44974, 44975, 44976, 44977, 44978, 44979, 44980 were selected from the ORFs having the nucleotide sequence represented by SEQ ID NO: 3529.

The probes having the nucleotide sequence represented by SEQ ID NOS: 44981, 44982, 44983, 44984, 44985, 44986, 44987, 44988, 44989, 44990, 44991 were selected from the ORFs having the nucleotide sequence represented by SEQ ID NO: 3530.

The probes having the nucleotide sequence represented by SEQ ID NOS: 44992, 44993, 44994, 44995, 44996, 44997, 44998, 44999, 45000, 45001, 45002 were selected from the ORFs having the nucleotide sequence represented by SEQ ID NO: 3531.

The probes having the nucleotide sequence represented by SEQ ID NOS: 45003, 45004, 45005, 45006, 45007, 45008, 45009, 45010, 45011, 45012, 45013 were selected from the ORFs having the nucleotide sequence represented by SEQ ID NO: 3532.

The probes having the nucleotide sequence represented by SEQ ID NOS: 45014, 45015, 45016, 45017, 45018, 45019, 45020, 45021, 45022, 45023, 45024 were selected from the ORFs having the nucleotide sequence represented by SEQ ID NO: 3533.

The probes having the nucleotide sequence represented by SEQ ID NOS: 45025, 45026, 45027, 45028, 45029, 45030, 45031, 45032, 45033, 45034, 45035 were selected from the ORFs having the nucleotide sequence represented by SEQ ID NO: 3534.

The probes having the nucleotide sequence represented by SEQ ID NOS: 45036, 45037, 45038, 45039, 45040, 45041, 45042, 45043, 45044, 45045, 45046 were selected from the ORFs having the nucleotide sequence represented by SEQ ID NO: 3535.

The probes having the nucleotide sequence represented by SEQ ID NOS: 45047, 45048, 45049, 45050, 45051, 45052, 45053, 45054, 45055, 45056, 45057 were selected from the ORFs having the nucleotide sequence represented by SEQ ID NO: 3536.

The probes having the nucleotide sequence represented by SEQ ID NOS: 45058, 45059, 45060, 45061, 45062, 45063, 45064, 45065, 45066, 45067, 45068 were selected from the ORFs having the nucleotide sequence represented by SEQ ID NO: 3537.

The probes having the nucleotide sequence represented by SEQ ID NOS: 45069, 45070, 45071, 45072, 45073, 45074, 45075, 45076, 45077, 45078, 45079 were selected from the ORFs having the nucleotide sequence represented by SEQ ID NO: 3538.

The probes having the nucleotide sequence represented by SEQ ID NOS: 45080, 45081, 45082, 45083, 45084, 45085, 45086, 45087, 45088, 45089, 45090 were selected from the ORFs having the nucleotide sequence represented by SEQ ID NO: 3539.

The probes having the nucleotide sequence represented by SEQ ID NOS: 45091, 45092, 45093, 45094, 45095, 45096, 45097, 45098, 45099, 45100, 45101 were selected from the ORFs having the nucleotide sequence represented by SEQ ID NO: 3540.

The probes having the nucleotide sequence represented by SEQ ID NOS: 45102, 45103, 45104, 45105, 45106, 45107, 45108, 45109, 45110, 45111, 45112 were selected from the ORFs having the nucleotide sequence represented by SEQ ID NO: 3541.

The probes having the nucleotide sequence represented by SEQ ID NOS: 45113, 45114, 45115, 45116, 45117, 45118, 45119, 45120, 45121, 45122, 45123 were selected from the ORFs having the nucleotide sequence represented by SEQ ID NO: 3542.

The probes having the nucleotide sequence represented by SEQ ID NOS: 45124, 45125, 45126, 45127, 45128, 45129, 45130, 45131, 45132, 45133, 45134 were selected from the ORFs having the nucleotide sequence represented by SEQ ID NO: 3543.

The probes having the nucleotide sequence represented by SEQ ID NOS: 45135, 45136, 45137, 45138, 45139, 45140, 45141, 45142, 45143, 45144, 45145 were selected from the ORFs having the nucleotide sequence represented by SEQ ID NO: 3544.

The probes having the nucleotide sequence represented by SEQ ID NOS: 45146, 45147, 45148, 45149, 45150, 45151, 45152, 45153, 45154, 45155, 45156 were selected from the ORFs having the nucleotide sequence represented by SEQ ID NO: 3545.

The probes having the nucleotide sequence represented by SEQ ID NOS: 45157, 45158, 45159, 45160, 45161, 45162, 45163, 45164, 45165, 45166, 45167 were selected from the ORFs having the nucleotide sequence represented by SEQ ID NO: 3546.

The probes having the nucleotide sequence represented by SEQ ID NOS: 45168, 45169, 45170, 45171, 45172, 45173, 45174, 45175, 45176, 45177, 45178 were selected from the ORFs having the nucleotide sequence represented by SEQ ID NO: 3547.

The probes having the nucleotide sequence represented by SEQ ID NOS: 45179, 45180, 45181, 45182, 45183, 45184, 45185, 45186, 45187, 45188, 45189 were selected from the ORFs having the nucleotide sequence represented by SEQ ID NO: 3548.

The probes having the nucleotide sequence represented by SEQ ID NOS: 45190, 45191, 45192, 45193, 45194, 45195, 45196, 45197, 45198, 45199, 45200 were selected from the ORFs having the nucleotide sequence represented by SEQ ID NO: 3549.

The probes having the nucleotide sequence represented by SEQ ID NOS: 45201, 45202, 45203, 45204, 45205, 45206, 45207, 45208, 45209, 45210, 45211 were selected from the ORFs having the nucleotide sequence represented by SEQ ID NO: 3550.

The probes having the nucleotide sequence represented by SEQ ID NOS: 45212, 45213, 45214, 45215, 45216, 45217, 45218, 45219, 45220, 45221, 45222 were selected from the ORFs having the nucleotide sequence represented by SEQ ID NO: 3551.

The probes having the nucleotide sequence represented by SEQ ID NOS: 45223, 45224, 45225, 45226, 45227, 45228, 45229, 45230, 45231, 45232, 45233 were selected from the ORFs having the nucleotide sequence represented by SEQ ID NO: 3552.

The probes having the nucleotide sequence represented by SEQ ID NOS: 45234, 45235, 45236, 45237, 45238, 45239, 45240, 45241, 45242, 45243, 45244 were selected from the ORFs having the nucleotide sequence represented by SEQ ID NO: 3553.

The probes having the nucleotide sequence represented by SEQ ID NOS: 45245, 45246, 45247, 45248, 45249, 45250, 45251, 45252, 45253, 45254, 45255 were selected from the ORFs having the nucleotide sequence represented by SEQ ID NO: 3554.

The probes having the nucleotide sequence represented by SEQ ID NOS: 45256, 45257, 45258, 45259, 45260, 45261, 45262, 45263, 45264, 45265, 45266 were selected from the ORFs having the nucleotide sequence represented by SEQ ID NO: 3555.

The probes having the nucleotide sequence represented by SEQ ID NOS: 45267, 45268, 45269, 45270, 45271, 45272, 45273, 45274, 45275, 45276, 45277 were selected from the ORFs having the nucleotide sequence represented by SEQ ID NO: 3556.

The probes having the nucleotide sequence represented by SEQ ID NOS: 45278, 45279, 45280, 45281, 45282, 45283, 45284, 45285, 45286, 45287, 45288 were selected from the ORFs having the nucleotide sequence represented by SEQ ID NO: 3557.

The probes having the nucleotide sequence represented by SEQ ID NOS: 45289, 45290, 45291, 45292, 45293, 45294, 45295, 45296, 45297, 45298, 45299 were selected from the ORFs having the nucleotide sequence represented by SEQ ID NO: 3558.

The probes having the nucleotide sequence represented by SEQ ID NOS: 45300, 45301, 45302, 45303, 45304, 45305, 45306, 45307, 45308, 45309, 45310 were selected from the ORFs having the nucleotide sequence represented by SEQ ID NO: 3559.

The probes having the nucleotide sequence represented by SEQ ID NOS: 45311, 45312, 45313, 45314, 45315, 45316, 45317, 45318, 45319, 45320, 45321 were selected from the ORFs having the nucleotide sequence represented by SEQ ID NO: 3560.

The probes having the nucleotide sequence represented by SEQ ID NOS: 45322, 45323, 45324, 45325, 45326, 45327, 45328, 45329, 45330, 45331, 45332 were selected from the ORFs having the nucleotide sequence represented by SEQ ID NO: 3561.

The probes having the nucleotide sequence represented by SEQ ID NOS: 45333, 45334, 45335, 45336, 45337, 45338, 45339, 45340, 45341, 45342, 45343 were selected from the ORFs having the nucleotide sequence represented by SEQ ID NO: 3562.

The probes having the nucleotide sequence represented by SEQ ID NOS: 45344, 45345, 45346, 45347, 45348, 45349, 45350, 45351, 45352, 45353, 45354 were selected from the ORFs having the nucleotide sequence represented by SEQ ID NO: 3563.

The probes having the nucleotide sequence represented by SEQ ID NOS: 45355, 45356, 45357, 45358, 45359, 45360, 45361, 45362, 45363, 45364, 45365 were selected from the ORFs having the nucleotide sequence represented by SEQ ID NO: 3564.

The probes having the nucleotide sequence represented by SEQ ID NOS: 45366, 45367, 45368, 45369, 45370, 45371, 45372, 45373, 45374, 45375, 45376 were selected from the ORFs having the nucleotide sequence represented by SEQ ID NO: 3565.

The probes having the nucleotide sequence represented by SEQ ID NOS: 45377, 45378, 45379, 45380, 45381, 45382, 45383, 45384, 45385, 45386, 45387 were selected from the ORFs having the nucleotide sequence represented by SEQ ID NO: 3566.

The probes having the nucleotide sequence represented by SEQ ID NOS: 45388, 45389, 45390, 45391, 45392, 45393, 45394, 45395, 45396, 45397, 45398 were selected from the ORFs having the nucleotide sequence represented by SEQ ID NO: 3567.

The probes having the nucleotide sequence represented by SEQ ID NOS: 45399, 45400, 45401, 45402, 45403, 45404, 45405, 45406, 45407, 45408, 45409 were selected from the ORFs having the nucleotide sequence represented by SEQ ID NO: 3568.

The probes having the nucleotide sequence represented by SEQ ID NOS: 45410, 45411, 45412, 45413, 45414, 45415, 45416, 45417, 45418, 45419, 45420 were selected from the ORFs having the nucleotide sequence represented by SEQ ID NO: 3569.

The probes having the nucleotide sequence represented by SEQ ID NOS: 45421, 45422, 45423, 45424, 45425, 45426, 45427, 45428, 45429, 45430, 45431 were selected from the ORFs having the nucleotide sequence represented by SEQ ID NO: 3570.

The probes having the nucleotide sequence represented by SEQ ID NOS: 45432, 45433, 45434, 45435, 45436, 45437, 45438, 45439, 45440, 45441, 45442 were selected from the ORFs having the nucleotide sequence represented by SEQ ID NO: 3571.

The probes having the nucleotide sequence represented by SEQ ID NOS: 45443, 45444, 45445, 45446, 45447, 45448, 45449, 45450, 45451, 45452, 45453 were selected from the ORFs having the nucleotide sequence represented by SEQ ID NO: 3572.

The probes having the nucleotide sequence represented by SEQ ID NOS: 45454, 45455, 45456, 45457, 45458, 45459, 45460, 45461, 45462, 45463, 45464 were selected from the ORFs having the nucleotide sequence represented by SEQ ID NO: 3573.

The probes having the nucleotide sequence represented by SEQ ID NOS: 45465, 45466, 45467, 45468, 45469, 45470, 45471, 45472, 45473, 45474, 45475 were selected from the ORFs having the nucleotide sequence represented by SEQ ID NO: 3574.

The probes having the nucleotide sequence represented by SEQ ID NOS: 45476, 45477, 45478, 45479, 45480, 45481, 45482, 45483, 45484, 45485, 45486 were selected from the ORFs having the nucleotide sequence represented by SEQ ID NO: 3575.

The probes having the nucleotide sequence represented by SEQ ID NOS: 45487, 45488, 45489, 45490, 45491, 45492, 45493, 45494, 45495, 45496, 45497 were selected from the ORFs having the nucleotide sequence represented by SEQ ID NO: 3576.

The probes having the nucleotide sequence represented by SEQ ID NOS: 45498, 45499, 45500, 45501, 45502, 45503, 45504, 45505, 45506, 45507, 45508 were selected from the ORFs having the nucleotide sequence represented by SEQ ID NO: 3577.

The probes having the nucleotide sequence represented by SEQ ID NOS: 45509, 45510, 45511, 45512, 45513, 45514, 45515, 45516, 45517, 45518, 45519 were selected from the ORFs having the nucleotide sequence represented by SEQ ID NO: 3578.

The probes having the nucleotide sequence represented by SEQ ID NOS: 45520, 45521, 45522, 45523, 45524, 45525, 45526, 45527, 45528, 45529, 45530 were selected from the ORFs having the nucleotide sequence represented by SEQ ID NO: 3579.

The probes having the nucleotide sequence represented by SEQ ID NOS: 45531, 45532, 45533, 45534, 45535, 45536, 45537, 45538, 45539, 45540, 45541 were selected from the ORFs having the nucleotide sequence represented by SEQ ID NO: 3580.

The probes having the nucleotide sequence represented by SEQ ID NOS: 45542, 45543, 45544, 45545, 45546, 45547, 45548, 45549, 45550, 45551, 45552 were selected from the ORFs having the nucleotide sequence represented by SEQ ID NO: 3581.

The probes having the nucleotide sequence represented by SEQ ID NOS: 45553, 45554, 45555, 45556, 45557, 45558, 45559, 45560, 45561, 45562, 45563 were selected from the ORFs having the nucleotide sequence represented by SEQ ID NO: 3582.

The probes having the nucleotide sequence represented by SEQ ID NOS: 45564, 45565, 45566, 45567, 45568, 45569, 45570, 45571, 45572, 45573, 45574 were selected from the ORFs having the nucleotide sequence represented by SEQ ID NO: 3583.

The probes having the nucleotide sequence represented by SEQ ID NOS: 45575, 45576, 45577, 45578, 45579, 45580, 45581, 45582, 45583, 45584, 45585 were selected from the ORFs having the nucleotide sequence represented by SEQ ID NO: 3584.

The probes having the nucleotide sequence represented by SEQ ID NOS: 45586, 45587, 45588, 45589, 45590, 45591, 45592, 45593, 45594, 45595, 45596 were selected from the ORFs having the nucleotide sequence represented by SEQ ID NO: 3585.

The probes having the nucleotide sequence represented by SEQ ID NOS: 45597, 45598, 45599, 45600, 45601, 45602, 45603, 45604, 45605, 45606, 45607 were selected from the ORFs having the nucleotide sequence represented by SEQ ID NO: 3586.

The probes having the nucleotide sequence represented by SEQ ID NOS: 45608, 45609, 45610, 45611, 45612, 45613, 45614, 45615, 45616, 45617, 45618 were selected from the ORFs having the nucleotide sequence represented by SEQ ID NO: 3587.

The probes having the nucleotide sequence represented by SEQ ID NOS: 45619, 45620, 45621, 45622, 45623, 45624, 45625, 45626, 45627, 45628, 45629 were selected from the ORFs having the nucleotide sequence represented by SEQ ID NO: 3588.

The probes having the nucleotide sequence represented by SEQ ID NOS: 45630, 45631, 45632, 45633, 45634, 45635, 45636, 45637, 45638, 45639, 45640 were selected from the ORFs having the nucleotide sequence represented by SEQ ID NO: 3589.

The probes having the nucleotide sequence represented by SEQ ID NOS: 45641, 45642, 45643, 45644, 45645, 45646, 45647, 45648, 45649, 45650, 45651 were selected from the ORFs having the nucleotide sequence represented by SEQ ID NO: 3590.

The probes having the nucleotide sequence represented by SEQ ID NOS: 45652, 45653, 45654, 45655, 45656, 45657, 45658, 45659, 45660, 45661, 45662 were selected from the ORFs having the nucleotide sequence represented by SEQ ID NO: 3591.

The probes having the nucleotide sequence represented by SEQ ID NOS: 45663, 45664, 45665, 45666, 45667, 45668, 45669, 45670, 45671, 45672, 45673 were selected from the ORFs having the nucleotide sequence represented by SEQ ID NO: 3592.

The probes having the nucleotide sequence represented by SEQ ID NOS: 45674, 45675, 45676, 45677, 45678, 45679, 45680, 45681, 45682, 45683, 45684 were selected from the ORFs having the nucleotide sequence represented by SEQ ID NO: 3593.

The probes having the nucleotide sequence represented by SEQ ID NOS: 45685, 45686, 45687, 45688, 45689, 45690, 45691, 45692, 45693, 45694, 45695 were selected from the ORFs having the nucleotide sequence represented by SEQ ID NO: 3594.

The probes having the nucleotide sequence represented by SEQ ID NOS: 45696, 45697, 45698, 45699, 45700, 45701, 45702, 45703, 45704, 45705, 45706 were selected from the ORFs having the nucleotide sequence represented by SEQ ID NO: 3595.

The probes having the nucleotide sequence represented by SEQ ID NOS: 45707, 45708, 45709, 45710, 45711, 45712, 45713, 45714, 45715, 45716, 45717 were selected from the ORFs having the nucleotide sequence represented by SEQ ID NO: 3596.

The probes having the nucleotide sequence represented by SEQ ID NOS: 45718, 45719, 45720, 45721, 45722, 45723, 45724, 45725, 45726, 45727, 45728 were selected from the ORFs having the nucleotide sequence represented by SEQ ID NO: 3597.

The probes having the nucleotide sequence represented by SEQ ID NOS: 45729, 45730, 45731, 45732, 45733, 45734, 45735, 45736, 45737, 45738, 45739 were selected from the ORFs having the nucleotide sequence represented by SEQ ID NO: 3598.

The probes having the nucleotide sequence represented by SEQ ID NOS: 45740, 45741, 45742, 45743, 45744, 45745,

45746, 45747, 45748, 45749, 45750 were selected from the ORFs having the nucleotide sequence represented by SEQ ID NO: 3599.

The probes having the nucleotide sequence represented by SEQ ID NOS: 45751, 45752, 45753, 45754, 45755, 45756, 45757, 45758, 45759, 45760, 45761 were selected from the ORFs having the nucleotide sequence represented by SEQ ID NO: 3600.

The probes having the nucleotide sequence represented by SEQ ID NOS: 45762, 45763, 45764, 45765, 45766, 45767, 45768, 45769, 45770, 45771, 45772 were selected from the ORFs having the nucleotide sequence represented by SEQ ID NO: 3601.

The probes having the nucleotide sequence represented by SEQ ID NOS: 45773, 45774, 45775, 45776, 45777, 45778, 45779, 45780, 45781, 45782, 45783 were selected from the ORFs having the nucleotide sequence represented by SEQ ID NO: 3602.

The probes having the nucleotide sequence represented by SEQ ID NOS: 45784, 45785, 45786, 45787, 45788, 45789, 45790, 45791, 45792, 45793, 45794 were selected from the ORFs having the nucleotide sequence represented by SEQ ID NO: 3603.

The probes having the nucleotide sequence represented by SEQ ID NOS: 45795, 45796, 45797, 45798, 45799, 45800, 45801, 45802, 45803, 45804, 45805 were selected from the ORFs having the nucleotide sequence represented by SEQ ID NO: 3604.

The probes having the nucleotide sequence represented by SEQ ID NOS: 45806, 45807, 45808, 45809, 45810, 45811, 45812, 45813, 45814, 45815, 45816 were selected from the ORFs having the nucleotide sequence represented by SEQ ID NO: 3605.

The probes having the nucleotide sequence represented by SEQ ID NOS: 45817, 45818, 45819, 45820, 45821, 45822, 45823, 45824, 45825, 45826, 45827 were selected from the ORFs having the nucleotide sequence represented by SEQ ID NO: 3606.

The probes having the nucleotide sequence represented by SEQ ID NOS: 45828, 45829, 45830, 45831, 45832, 45833, 45834, 45835, 45836, 45837, 45838 were selected from the ORFs having the nucleotide sequence represented by SEQ ID NO: 3607.

The probes having the nucleotide sequence represented by SEQ ID NOS: 45839, 45840, 45841, 45842, 45843, 45844, 45845, 45846, 45847, 45848, 45849 were selected from the ORFs having the nucleotide sequence represented by SEQ ID NO: 3608.

The probes having the nucleotide sequence represented by SEQ ID NOS: 45850, 45851, 45852, 45853, 45854, 45855, 45856, 45857, 45858, 45859, 45860 were selected from the ORFs having the nucleotide sequence represented by SEQ ID NO: 3609.

The probes having the nucleotide sequence represented by SEQ ID NOS: 45861, 45862, 45863, 45864, 45865, 45866, 45867, 45868, 45869, 45870, 45871 were selected from the ORFs having the nucleotide sequence represented by SEQ ID NO: 3610.

The probes having the nucleotide sequence represented by SEQ ID NOS: 45872, 45873, 45874, 45875, 45876, 45877, 45878, 45879, 45880, 45881, 45882 were selected from the ORFs having the nucleotide sequence represented by SEQ ID NO: 3611.

The probes having the nucleotide sequence represented by SEQ ID NOS: 45883, 45884, 45885, 45886, 45887, 45888, 45889, 45890, 45891, 45892, 45893 were selected from the ORFs having the nucleotide sequence represented by SEQ ID NO: 3612.

The probes having the nucleotide sequence represented by SEQ ID NOS: 45894, 45895, 45896, 45897, 45898, 45899, 45900, 45901, 45902, 45903, 45904 were selected from the ORFs having the nucleotide sequence represented by SEQ ID NO: 3613.

The probes having the nucleotide sequence represented by SEQ ID NOS: 45905, 45906, 45907, 45908, 45909, 45910, 45911, 45912, 45913, 45914, 45915 were selected from the ORFs having the nucleotide sequence represented by SEQ ID NO: 3614.

The probes having the nucleotide sequence represented by SEQ ID NOS: 45916, 45917, 45918, 45919, 45920, 45921, 45922, 45923, 45924, 45925, 45926 were selected from the ORFs having the nucleotide sequence represented by SEQ ID NO: 3615.

The probes having the nucleotide sequence represented by SEQ ID NOS: 45927, 45928, 45929, 45930, 45931, 45932, 45933, 45934, 45935, 45936, 45937 were selected from the ORFs having the nucleotide sequence represented by SEQ ID NO: 3616.

The probes having the nucleotide sequence represented by SEQ ID NOS: 45938, 45939, 45940, 45941, 45942, 45943, 45944, 45945, 45946, 45947, 45948 were selected from the ORFs having the nucleotide sequence represented by SEQ ID NO: 3617.

The probes having the nucleotide sequence represented by SEQ ID NOS: 45949, 45950, 45951, 45952, 45953, 45954, 45955, 45956, 45957, 45958, 45959 were selected from the ORFs having the nucleotide sequence represented by SEQ ID NO: 3618.

The probes having the nucleotide sequence represented by SEQ ID NOS: 45960, 45961, 45962, 45963, 45964, A5965, 45966, 45967, 45968, 45969, 45970 were selected from the ORFs having the nucleotide sequence represented by SEQ ID NO: 3619.

The probes having the nucleotide sequence represented by SEQ ID NOS: 45971, 45972, 45973, 45974, 45975, 45976, 45977, 45978, 45979, 45980, 45981 were selected from the ORFs having the nucleotide sequence represented by SEQ ID NO: 3620.

The probes having the nucleotide sequence represented by SEQ ID NOS: 45982, 45983, 45984, 45985, 45986, 45987, 45988, 45989, 45990, 45991, 45992 were selected from the ORFs having the nucleotide sequence represented by SEQ ID NO: 3621.

The probes having the nucleotide sequence represented by SEQ ID NOS: 45993, 45994, 45995, 45996, 45997, 45998, 45999, 46000, 46001, 46002, 46003 were selected from the ORFs having the nucleotide sequence represented by SEQ ID NO: 3622.

The probes having the nucleotide sequence represented by SEQ ID NOS: 46004, 46005, 46006, 46007, 46008, 46009, 46010, 46011, 46012, 46013, 46014 were selected from the ORFs having the nucleotide sequence represented by SEQ ID NO: 3623.

The probes having the nucleotide sequence represented by SEQ ID NOS: 46015, 46016, 46017, 46018, 46019, 46020, 46021, 46022, 46023, 46024, 46025 were selected from the ORFs having the nucleotide sequence represented by SEQ ID NO: 3624.

The probes having the nucleotide sequence represented by SEQ ID NOS: 46026, 46027, 46028, 46029, 46030, 46031, 46032, 46033, 46034, 46035, 46036 were selected from the ORFs having the nucleotide sequence represented by SEQ ID NO: 3625.

The probes having the nucleotide sequence represented by SEQ ID NOS: 46037, 46038, 46039, 46040, 46041, 46042, 46043, 46044, 46045, 46046, 46047 were selected from the ORFs having the nucleotide sequence represented by SEQ ID NO: 3626.

The probes having the nucleotide sequence represented by SEQ ID NOS: 46048, 46049, 46050, 46051, 46052, 46053, 46054, 46055, 46056, 46057, 46058 were selected from the ORFs having the nucleotide sequence represented by SEQ ID NO: 3627.

The probes having the nucleotide sequence represented by SEQ ID NOS: 46059, 46060, 46061, 46062, 46063, 46064, 46065, 46066, 46067, 46068, 46069 were selected from the ORFs having the nucleotide sequence represented by SEQ ID NO: 3628.

The probes having the nucleotide sequence represented by SEQ ID NOS: 46070, 46071, 46072, 46073, 46074, 46075, 46076, 46077, 46078, 46079, 46080 were selected from the ORFs having the nucleotide sequence represented by SEQ ID NO: 3629.

The probes having the nucleotide sequence represented by SEQ ID NOS: 46081, 46082, 46083, 46084, 46085, 46086, 46087, 46088, 46089, 46090, 46091 were selected from the ORFs having the nucleotide sequence represented by SEQ ID NO: 3630.

The probes having the nucleotide sequence represented by SEQ ID NOS: 46092, 46093, 46094, 46095, 46096, 46097, 46098, 46099, 46100, 46101, 46102 were selected from the ORFs having the nucleotide sequence represented by SEQ ID NO: 3631.

The probes having the nucleotide sequence represented by SEQ ID NOS: 46103, 46104, 46105, 46106, 46107, 46108, 46109, 46110, 46111, 46112, 46113 were selected from the ORFs having the nucleotide sequence represented by SEQ ID NO: 3632.

The probes having the nucleotide sequence represented by SEQ ID NOS: 46114, 46115, 46116, 46117, 46118, 46119, 46120, 46121, 46122, 46123, 46124 were selected from the ORFs having the nucleotide sequence represented by SEQ ID NO: 3633.

The probes having the nucleotide sequence represented by SEQ ID NOS: 46125, 46126, 46127, 46128, 46129, 46130, 46131, 46132, 46133, 46134, 46135 were selected from the ORFs having the nucleotide sequence represented by SEQ ID NO: 3634.

The probes having the nucleotide sequence represented by SEQ ID NOS: 46136, 46137, 46138, 46139, 46140, 46141, 46142, 46143, 46144, 46145, 46146 were selected from the ORFs having the nucleotide sequence represented by SEQ ID NO: 3635.

The probes having the nucleotide sequence represented by SEQ ID NOS: 46147, 46148, 46149, 46150, 46151, 46152, 46153, 46154, 46155, 46156, 46157 were selected from the ORFs having the nucleotide sequence represented by SEQ ID NO: 3636.

The probes having the nucleotide sequence represented by SEQ ID NOS: 46158, 46159, 46160, 46161, 46162, 46163, 46164, 46165, 46166, 46167, 46168, 46169, 46170, 46171, 46172, 46173, 46174, 46175, 46176, 46177, 46178, 46179 were selected from the ORFs having the nucleotide sequence represented by SEQ ID NO: 3637.

The probes having the nucleotide sequence represented by SEQ ID NOS: 46180, 46181, 46182, 46183, 46184, 46185, 46186, 46187, 46188, 46189, 46190 were selected from the ORFs having the nucleotide sequence represented by SEQ ID NO: 3638.

The probes having the nucleotide sequence represented by SEQ ID NOS: 46191, 46192, 46193, 46194, 46195, 46196, 46197, 46198, 46199, 46200, 46201 were selected from the ORFs having the nucleotide sequence represented by SEQ ID NO: 3639.

The probes having the nucleotide sequence represented by SEQ ID NOS: 46202, 46203, 46204, 46205, 46206, 46207, 46208, 46209, 46210, 46211, 46212 were selected from the ORFs having the nucleotide sequence represented by SEQ ID NO: 3640.

The probes having the nucleotide sequence represented by SEQ ID NOS: 46213, 46214, 46215, 46216, 46217, 46218, 46219, 46220, 46221, 46222, 46223 were selected from the ORFs having the nucleotide sequence represented by SEQ ID NO: 3641.

The probes having the nucleotide sequence represented by SEQ ID NOS: 46224, 46225, 46226, 46227, 46228, 46229, 46230, 46231, 46232, 46233, 46234 were selected from the ORFs having the nucleotide sequence represented by SEQ ID NO: 3642.

The probes having the nucleotide sequence represented by SEQ ID NOS: 46235, 46236, 46237, 46238, 46239, 46240, 46241, 46242, 46243, 46244, 46245 were selected from the ORFs having the nucleotide sequence represented by SEQ ID NO: 3643.

The probes having the nucleotide sequence represented by SEQ ID NOS: 46246, 46247, 46248, 46249, 46250, 46251, 46252, 46253, 46254, 46255, 46256 were selected from the ORFs having the nucleotide sequence represented by SEQ ID NO: 3644.

The probes having the nucleotide sequence represented by SEQ ID NOS: 46257, 46258, 46259, 46260, 46261, 46262, 46263, 46264, 46265, 46266, 46267 were selected from the ORFs having the nucleotide sequence represented by SEQ ID NO: 3645.

The probes having the nucleotide sequence represented by SEQ ID NOS: 46268, 46269, 46270, 46271, 46272, 46273, 46274, 46275, 46276, 46277, 46278 were selected from the ORFs having the nucleotide sequence represented by SEQ ID NO: 3646.

The probes having the nucleotide sequence represented by SEQ ID NOS: 46279, 46280, 46281, 46282, 46283, 46284, 46285, 46286, 46287, 46288, 46289 were selected from the ORFs having the nucleotide sequence represented by SEQ ID NO: 3647.

The probes having the nucleotide sequence represented by SEQ ID NOS: 46290, 46291, 46292, 46293, 46294, 46295, 46296, 46297, 46298, 46299, 46300 were selected from the ORFs having the nucleotide sequence represented by SEQ ID NO: 3648.

The probes having the nucleotide sequence represented by SEQ ID NOS: 46301, 46302, 46303, 46304, 46305, 46306, 46307, 46308, 46309, 46310, 46311 were selected from the ORFs having the nucleotide sequence represented by SEQ ID NO: 3649.

The probes having the nucleotide sequence represented by SEQ ID NOS: 46312, 46313, 46314, 46315, 46316, 46317, 46318, 46319, 46320, 46321, 46322 were selected from the ORFs having the nucleotide sequence represented by SEQ ID NO: 3650.

The probes having the nucleotide sequence represented by SEQ ID NOS: 46323, 46324, 46325, 46326, 46327, 46328, 46329, 46330, 46331, 46332, 46333 were selected from the ORFs having the nucleotide sequence represented by SEQ ID NO: 3651.

The probes having the nucleotide sequence represented by SEQ ID NOS: 46334, 46335, 46336, 46337, 46338, 46339, 46340, 46341, 46342, 46343, 46344 were selected from the ORFs having the nucleotide sequence represented by SEQ ID NO: 3652.

The probes having the nucleotide sequence represented by SEQ ID NOS: 46345, 46346, 46347, 46348, 46349, 46350, 46351, 46352, 46353, 46354, 46355 were selected from the ORFs having the nucleotide sequence represented by SEQ ID NO: 3653.

The probes having the nucleotide sequence represented by SEQ ID NOS: 46356, 46357, 46358, 46359, 46360, 46361, 46362, 46363, 46364, 46365, 46366 were selected from the ORFs having the nucleotide sequence represented by SEQ ID NO: 3654.

The probes having the nucleotide sequence represented by SEQ ID NOS: 46367, 46368, 46369, 46370, 46371, 46372, 46373, 46374, 46375, 46376, 46377 were selected from the ORFs having the nucleotide sequence represented by SEQ ID NO: 3655.

The probes having the nucleotide sequence represented by SEQ ID NOS: 46378, 46379, 46380, 46381, 46382, 46383, 46384, 46385, 46386, 46387, 46388 were selected from the ORFs having the nucleotide sequence represented by SEQ ID NO: 3656.

The probes having the nucleotide sequence represented by SEQ ID NOS: 46389, 46390, 46391, 46392, 46393, 46394, 46395, 46396, 46397, 46398, 46399 were selected from the ORFs having the nucleotide sequence represented by SEQ ID NO: 3657.

The probes having the nucleotide sequence represented by SEQ ID NOS: 46400, 46401, 46402, 46403, 46404, 46405, 46406, 46407, 46408, 46409, 46410 were selected from the ORFs having the nucleotide sequence represented by SEQ ID NO: 3658.

The probes having the nucleotide sequence represented by SEQ ID NOS: 46411, 46412, 46413, 46414, 46415, 46416, 46417, 46418, 46419, 46420, 46421 were selected from the ORFs having the nucleotide sequence represented by SEQ ID NO: 3659.

The probes having the nucleotide sequence represented by SEQ ID NOS: 46422, 46423, 46424, 46425, 46426, 46427, 46428, 46429, 46430, 46431, 46432 were selected from the ORFs having the nucleotide sequence represented by SEQ ID NO: 3660.

The probes having the nucleotide sequence represented by SEQ ID NOS: 46433, 46434, 46435, 46436, 46437, 46438, 46439, 46440, 46441, 46442, 46443 were selected from the ORFs having the nucleotide sequence represented by SEQ ID NO: 3661.

The probes having the nucleotide sequence represented by SEQ ID NOS: 46444, 46445, 46446, 46447, 46448, 46449, 46450, 46451, 46452, 46453, 46454 were selected from the ORFs having the nucleotide sequence represented by SEQ ID NO: 3662.

The probes having the nucleotide sequence represented by SEQ ID NOS: 46455, 46456, 46457, 46458, 46459, 46460, 46461, 46462, 46463, 46464, 46465 were selected from the ORFs having the nucleotide sequence represented by SEQ ID NO: 3663.

The probes having the nucleotide sequence represented by SEQ ID NOS: 46466, 46467, 46468, 46469, 46470, 46471, 46472, 46473, 46474, 46475, 46476 were selected from the ORFs having the nucleotide sequence represented by SEQ ID NO: 3664.

The probes having the nucleotide sequence represented by SEQ ID NOS: 46477, 46478, 46479, 46480, 46481, 46482, 46483, 46484, 46485, 46486, 46487 were selected from the ORFs having the nucleotide sequence represented by SEQ ID NO: 3665.

The probes having the nucleotide sequence represented by SEQ ID NOS: 46488, 46489, 46490, 46491, 46492, 46493, 46494, 46495, 46496, 46497, 46498 were selected from the ORFs having the nucleotide sequence represented by SEQ ID NO: 3666.

The probes having the nucleotide sequence represented by SEQ ID NOS: 46499, 46500, 46501, 46502, 46503, 46504, 46505, 46506, 46507, 46508, 46509 were selected from the ORFs having the nucleotide sequence represented by SEQ ID NO: 3667.

The probes having the nucleotide sequence represented by SEQ ID NOS: 46510, 46511, 46512, 46513, 46514, 46515, 46516, 46517, 46518, 46519, 46520 were selected from the ORFs having the nucleotide sequence represented by SEQ ID NO: 3668.

The probes having the nucleotide sequence represented by SEQ ID NOS: 46521, 46522, 46523, 46524, 46525, 46526, 46527, 46528, 46529, 46530, 46531 were selected from the ORFs having the nucleotide sequence represented by SEQ ID NO: 3669.

The probes having the nucleotide sequence represented by SEQ ID NOS: 46532, 46533, 46534, 46535, 46536, 46537, 46538, 46539, 46540, 46541, 46542 were selected from the ORFs having the nucleotide sequence represented by SEQ ID NO: 3670.

The probes having the nucleotide sequence represented by SEQ ID NOS: 46543, 46544, 46545, 46546, 46547, 46548, 46549, 46550, 46551, 46552, 46553 were selected from the ORFs having the nucleotide sequence represented by SEQ ID NO: 3671.

The probes having the nucleotide sequence represented by SEQ ID NOS: 46554, 46555, 46556, 46557, 46558, 46559, 46560, 46561, 46562, 46563, 46564 were selected from the ORFs having the nucleotide sequence represented by SEQ ID NO: 3672.

The probes having the nucleotide sequence represented by SEQ ID NOS: 46565, 46566, 46567, 46568, 46569, 46570, 46571, 46572, 46573, 46574, 46575 were selected from the ORFs having the nucleotide sequence represented by SEQ ID NO: 3673.

The probes having the nucleotide sequence represented by SEQ ID NOS: 46576, 46577, 46578, 46579, 46580, 46581, 46582, 46583, 46584, 46585, 46586 were selected from the ORFs having the nucleotide sequence represented by SEQ ID NO: 3674.

The probes having the nucleotide sequence represented by SEQ ID NOS: 46587, 46588, 46589, 46590, 46591, 46592, 46593, 46594, 46595, 46596, 46597 were selected from the ORFs having the nucleotide sequence represented by SEQ ID NO: 3675.

The probes having the nucleotide sequence represented by SEQ ID NOS: 46598, 46599, 46600, 46601, 46602, 46603, 46604, 46605, 46606, 46607, 46608 were selected from the ORFs having the nucleotide sequence represented by SEQ ID NO: 3676.

The probes having the nucleotide sequence represented by SEQ ID NOS: 46609, 46610, 46611, 46612, 46613, 46614, 46615, 46616, 46617, 46618, 46619 were selected from the ORFs having the nucleotide sequence represented by SEQ ID NO: 3677.

The probes having the nucleotide sequence represented by SEQ ID NOS: 46620, 46621, 46622, 46623, 46624, 46625, 46626, 46627, 46628, 46629, 46630 were selected from the ORFs having the nucleotide sequence represented by SEQ ID NO: 3678.

The probes having the nucleotide sequence represented by SEQ ID NOS: 46631, 46632, 46633, 46634, 46635, 46636, 46637, 46638, 46639, 46640, 46641 were selected from the ORFs having the nucleotide sequence represented by SEQ ID NO: 3679.

The probes having the nucleotide sequence represented by SEQ ID NOS: 46642, 46643, 46644, 46645, 46646, 46647, 46648, 46649, 46650, 46651, 46652 were selected from the ORFs having the nucleotide sequence represented by SEQ ID NO: 3680.

The probes having the nucleotide sequence represented by SEQ ID NOS: 46653, 46654, 46655, 46656, 46657, 46658, 46659, 46660, 46661, 46662, 46663 were selected from the ORFs having the nucleotide sequence represented by SEQ ID NO: 3681.

The probes having the nucleotide sequence represented by SEQ ID NOS: 46664, 46665, 46666, 46667, 46668, 46669, 46670, 46671, 46672, 46673, 46674 were selected from the ORFs having the nucleotide sequence represented by SEQ ID NO: 3682.

The probes having the nucleotide sequence represented by SEQ ID NOS: 46675, 46676, 46677, 46678, 46679, 46680, 46681, 46682, 46683, 46684, 46685 were selected from the ORFs having the nucleotide sequence represented by SEQ ID NO: 3683.

The probes having the nucleotide sequence represented by SEQ ID NOS: 46686, 46687, 46688, 46689, 46690, 46691, 46692, 46693, 46694, 46695, 46696 were selected from the ORFs having the nucleotide sequence represented by SEQ ID NO: 3684.

The probes having the nucleotide sequence represented by SEQ ID NOS: 46697, 46698, 46699, 46700, 46701, 46702, 46703, 46704, 46705, 46706, 46707 were selected from the ORFs having the nucleotide sequence represented by SEQ ID NO: 3685.

The probes having the nucleotide sequence represented by SEQ ID NOS: 46708, 46709, 46710, 46711, 46712, 46713, 46714, 46715, 46716, 46717, 46718 were selected from the ORFs having the nucleotide sequence represented by SEQ ID NO: 3686.

The probes having the nucleotide sequence represented by SEQ ID NOS: 46719, 46720, 46721, 46722, 46723, 46724, 46725, 46726, 46727, 46728, 46729 were selected from the ORFs having the nucleotide sequence represented by SEQ ID NO: 3687.

The probes having the nucleotide sequence represented by SEQ ID NOS: 46730, 46731, 46732, 46733, 46734, 46735, 46736, 46737, 46738, 46739, 46740 were selected from the ORFs having the nucleotide sequence represented by SEQ ID NO: 3688.

The probes having the nucleotide sequence represented by SEQ ID NOS: 46741, 46742, 46743, 46744, 46745, 46746, 46747, 46748, 46749, 46750, 46751 were selected from the ORFs having the nucleotide sequence represented by SEQ ID NO: 3689.

The probes having the nucleotide sequence represented by SEQ ID NOS: 46752, 46753, 46754, 46755, 46756, 46757, 46758, 46759, 46760, 46761, 46762 were selected from the ORFs having the nucleotide sequence represented by SEQ ID NO: 3690.

The probes having the nucleotide sequence represented by SEQ ID NOS: 46763, 46764, 46765, 46766, 46767, 46768, 46769, 46770, 46771, 46772, 46773 were selected from the ORFs having the nucleotide sequence represented by SEQ ID NO: 3691.

The probes having the nucleotide sequence represented by SEQ ID NOS: 46774, 46775, 46776, 46777, 46778, 46779, 46780, 46781, 46782, 46783, 46784 were selected from the ORFs having the nucleotide sequence represented by SEQ ID NO: 3692.

The probes having the nucleotide sequence represented by SEQ ID NOS: 46785, 46786, 46787, 46788, 46789, 46790, 46791, 46792, 46793, 46794, 46795 were selected from the ORFs having the nucleotide sequence represented by SEQ ID NO: 3693.

The probes having the nucleotide sequence represented by SEQ ID NOS: 46796, 46797, 46798, 46799, 46800, 46801, 46802, 46803, 46804, 46805, 46806 were selected from the ORFs having the nucleotide sequence represented by SEQ ID NO: 3694.

The probes having the nucleotide sequence represented by SEQ ID NOS: 46807, 46808, 46809, 46810, 46811, 46812, 46813, 46814, 46815, 46816, 46817 were selected from the ORFs having the nucleotide sequence represented by SEQ ID NO: 3695.

The probes having the nucleotide sequence represented by SEQ ID NOS: 46818, 46819, 46820, 46821, 46822, 46823, 46824, 46825, 46826, 46827, 46828 were selected from the ORFs having the nucleotide sequence represented by SEQ ID NO: 3696.

The probes having the nucleotide sequence represented by SEQ ID NOS: 46829, 46830, 46831, 46832, 46833, 46834, 46835, 46836, 46837, 46838, 46839 were selected from the ORFs having the nucleotide sequence represented by SEQ ID NO: 3697.

The probes having the nucleotide sequence represented by SEQ ID NOS: 46840, 46841, 46842, 46843, 46844, 46845, 46846, 46847, 46848, 46849, 46850 were selected from the ORFs having the nucleotide sequence represented by SEQ ID NO: 3698.

The probes having the nucleotide sequence represented by SEQ ID NOS: 46851, 46852, 46853, 46854, 46855, 46856, 46857, 46858, 46859, 46860, 46861 were selected from the ORFs having the nucleotide sequence represented by SEQ ID NO: 3699.

The probes having the nucleotide sequence represented by SEQ ID NOS: 46862, 46863, 46864, 46865, 46866, 46867, 46868, 46869, 46870, 46871, 46872 were selected from the ORFs having the nucleotide sequence represented by SEQ ID NO: 3700.

The probes having the nucleotide sequence represented by SEQ ID NOS: 46873, 46874, 46875, 46876, 46877, 46878, 46879, 46880, 46881, 46882, 46883 were selected from the ORFs having the nucleotide sequence represented by SEQ ID NO: 3701.

The probes having the nucleotide sequence represented by SEQ ID NOS: 46884, 46885, 46886, 46887, 46888, 46889, 46890, 46891, 46892, 46893, 46894 were selected from the ORFs having the nucleotide sequence represented by SEQ ID NO: 3702.

The probes having the nucleotide sequence represented by SEQ ID NOS: 46895, 46896, 46897, 46898, 46899, 46900, 46901, 46902, 46903, 46904, 46905 were selected from the ORFs having the nucleotide sequence represented by SEQ ID NO: 3703.

The probes having the nucleotide sequence represented by SEQ ID NOS: 46906, 46907, 46908, 46909, 46910, 46911, 46912, 46913, 46914, 46915, 46916, 46917, 46918, 46919, 46920, 46921, 46922, 46923, 46924, 46925, 46926, 46927 were selected from the ORFs having the nucleotide sequence represented by SEQ ID NO: 3704.

The probes having the nucleotide sequence represented by SEQ ID NOS: 46928, 46929, 46930, 46931, 46932, 46933, 46934, 46935, 46936, 46937, 46938 were selected from the ORFs having the nucleotide sequence represented by SEQ ID NO: 3705.

The probes having the nucleotide sequence represented by SEQ ID NOS: 46939, 46940, 46941, 46942, 46943, 46944, 46945, 46946, 46947, 46948, 46949 were selected from the ORFs having the nucleotide sequence represented by SEQ ID NO: 3706.

The probes having the nucleotide sequence represented by SEQ ID NOS: 46950, 46951, 46952, 46953, 46954, 46955, 46956, 46957, 46958, 46959, 46960 were selected from the ORFs having the nucleotide sequence represented by SEQ ID NO: 3707.

The probes having the nucleotide sequence represented by SEQ ID NOS: 46961, 46962, 46963, 46964, 46965, 46966, 46967, 46968, 46969, 46970, 46971 were selected from the ORFs having the nucleotide sequence represented by SEQ ID NO: 3708.

The probes having the nucleotide sequence represented by SEQ ID NOS: 46972, 46973, 46974, 46975, 46976, 46977, 46978, 46979, 46980, 46981, 46982 were selected from the ORFs having the nucleotide sequence represented by SEQ ID NO: 3709.

The probes having the nucleotide sequence represented by SEQ ID NOS: 46983, 46984, 46985, 46986, 46987, 46988, 46989, 46990, 46991, 46992, 46993 were selected from the ORFs having the nucleotide sequence represented by SEQ ID NO: 3710.

The probes having the nucleotide sequence represented by SEQ ID NOS: 46994, 46995, 46996, 46997, 46998, 46999, 47000, 47001, 47002, 47003, 47004 were selected from the ORFs having the nucleotide sequence represented by SEQ ID NO: 3711.

The probes having the nucleotide sequence represented by SEQ ID NOS: 47005, 47006, 47007, 47008, 47009, 47010, 47011, 47012, 47013, 47014, 47015 were selected from the ORFs having the nucleotide sequence represented by SEQ ID NO: 3712.

The probes having the nucleotide sequence represented by SEQ ID NOS: 47016, 47017, 47018, 47019, 47020, 47021, 47022, 47023, 47024, 47025, 47026 were selected from the ORFs having the nucleotide sequence represented by SEQ ID NO: 3713.

The probes having the nucleotide sequence represented by SEQ ID NOS: 47027, 47028, 47029, 47030, 47031, 47032, 47033, 47034, 47035, 47036, 47037, 47038, 47039, 47040, 47041, 47042, 47043, 47044, 47045, 47046, 47047, 47048 were selected from the ORFs having the nucleotide sequence represented by SEQ ID NO: 3714.

The probes having the nucleotide sequence represented by SEQ ID NOS: 47049, 47050, 47051, 47052, 47053, 47054, 47055, 47056, 47057, 47058, 47059 were selected from the ORFs having the nucleotide sequence represented by SEQ ID NO: 3715.

The probes having the nucleotide sequence represented by SEQ ID NOS: 47060, 47061, 47062, 47063, 47064, 47065, 47066, 47067, 47068, 47069, 47070 were selected from the ORFs having the nucleotide sequence represented by SEQ ID NO: 3716.

The probes having the nucleotide sequence represented by SEQ ID NOS: 47071, 47072, 47073, 47074, 47075, 47076, 47077, 47078, 47079, 47080, 47081 were selected from the ORFs having the nucleotide sequence represented by SEQ ID NO: 3717.

The probes having the nucleotide sequence represented by SEQ ID NOS: 47082, 47083, 470.84, 47085, 47086, 47087, 47088, 47089, 47090, 47091, 47092 were selected from the ORFs having the nucleotide sequence represented by SEQ ID NO: 3718.

The probes having the nucleotide sequence represented by SEQ ID NOS: 47093, 47094, 47095, 47096, 47097, 47098, 47099, 47100, 47101, 47102, 47103 were selected from the ORFs having the nucleotide sequence represented by SEQ ID NO: 3719.

The probes having the nucleotide sequence represented by SEQ ID NOS: 47104, 47105, 47106, 47107, 47108, 47109, 47110, 47111, 47112, 47113, 47114, 47115, 47116, 47117, 47118, 47119, 47120, 47121, 47122, 47123, 47124, 47125 were selected from the ORFs having the nucleotide sequence represented by SEQ ID NO: 3720.

The probes having the nucleotide sequence represented by SEQ ID NOS: 47126, 47127, 47128, 47129, 47130, 47131, 47132, 47133, 47134, 47135, 47136 were selected from the ORFs having the nucleotide sequence represented by SEQ ID NO: 3721.

The probes having the nucleotide sequence represented by SEQ ID NOS: 47137, 47138, 47139, 47140, 47141, 47142, 47143, 47144, 47145, 47146, 47147 were selected from the ORFs having the nucleotide sequence represented by SEQ ID NO: 3722.

The probes having the nucleotide sequence represented by SEQ ID NOS: 47148, 47149, 47150, 47151, 47152, 47153, 47154, 47155, 47156, 47157, 47158 were selected from the ORFs having the nucleotide sequence represented by SEQ ID NO: 3723.

The probes having the nucleotide sequence represented by SEQ ID NOS: 47159, 47160, 47161, 47162, 47163, 47164, 47165, 47166, 47167, 47168, 47169 were selected from the ORFs having the nucleotide sequence represented by SEQ ID NO: 3724.

The probes having the nucleotide sequence represented by SEQ ID NOS: 47170, 47171, 47172, 47173, 47174, 47175, 47176, 47177, 47178, 47179, 47180 were selected from the ORFs having the nucleotide sequence represented by SEQ ID NO: 3725.

The probes having the nucleotide sequence represented by SEQ ID NOS: 47181, 47182, 47183, 47184, 47185, 47186, 47187, 47188, 47189, 47190, 47191 were selected from the ORFs having the nucleotide sequence represented by SEQ ID NO: 3726.

The probes having the nucleotide sequence represented by SEQ ID NOS: 47192, 47193, 47194, 47195, 47196, 47197, 47198, 47199, 47200, 47201, 47202 were selected from the ORFs having the nucleotide sequence represented by SEQ ID NO: 3727.

The probes having the nucleotide sequence represented by SEQ ID NOS: 47203, 47204, 47205, 47206, 47207, 47208, 47209, 47210, 47211, 47212, 47213 were selected from the ORFs having the nucleotide sequence represented by SEQ ID NO: 3728.

The probes having the nucleotide sequence represented by SEQ ID NOS: 47214, 47215, 47216, 47217, 47218, 47219, 47220, 47221, 47222, 47223, 47224 were selected from the ORFs having the nucleotide sequence represented by SEQ ID NO: 3729.

The probes having the nucleotide sequence represented by SEQ ID NOS: 47225, 47226, 47227, 47228, 47229, 47230, 47231, 47232, 47233, 47234, 47235 were selected from the ORFs having the nucleotide sequence represented by SEQ ID NO: 3730.

The probes having the nucleotide sequence represented by SEQ ID NOS: 47236, 47237, 47238, 47239, 47240, 47241, 47242, 47243, 47244, 47245, 47246 were selected from the ORFs having the nucleotide sequence represented by SEQ ID NO: 3731.

The probes having the nucleotide sequence represented by SEQ ID NOS: 47247, 47248, 47249, 47250, 47251, 47252, 47253, 47254, 47255, 47256, 47257 were selected from the ORFs having the nucleotide sequence represented by SEQ ID NO: 3732.

The probes having the nucleotide sequence represented by SEQ ID NOS: 47258, 47259, 47260, 47261, 47262, 47263, 47264, 47265, 47266, 47267, 47268 were selected from the ORFs having the nucleotide sequence represented by SEQ ID NO: 3733.

The probes having the nucleotide sequence represented by SEQ ID NOS: 47269, 47270, 47271, 47272, 47273, 47274, 47275, 47276, 47277, 47278, 47279 were selected from the ORFs having the nucleotide sequence represented by SEQ ID NO: 3734.

The probes having the nucleotide sequence represented by SEQ ID NOS: 47280, 47281, 47282, 47283, 47284, 47285, 47286, 47287, 47288, 47289, 47290 were selected from the ORFs having the nucleotide sequence represented by SEQ ID NO: 3735.

The probes having the nucleotide sequence represented by SEQ ID NOS: 47291, 47292, 47293, 47294, 47295, 47296, 47297, 47298, 47299, 47300, 47301 were selected from the ORFs having the nucleotide sequence represented by SEQ ID NO: 3736.

The probes having the nucleotide sequence represented by SEQ ID NOS: 47302, 47303, 47304, 47305, 47306, 47307, 47308, 47309, 47310, 47311, 47312 were selected from the ORFs having the nucleotide sequence represented by SEQ ID NO: 3737.

The probes having the nucleotide sequence represented by SEQ ID NOS: 47313, 47314, 47315, 47316, 47317, 47318, 47319, 47320, 47321, 47322, 47323 were selected from the ORFs having the nucleotide sequence represented by SEQ ID NO: 3738.

The probes having the nucleotide sequence represented by SEQ ID NOS: 47324, 47325, 47326, 47327, 47328, 47329, 47330, 47331, 47332, 47333, 47334 were selected from the ORFs having the nucleotide sequence represented by SEQ ID NO: 3739.

The probes having the nucleotide sequence represented by SEQ ID NOS: 47335, 47336, 47337, 47338, 47339, 47340, 47341, 47342, 47343, 47344, 47345 were selected from the ORFs having the nucleotide sequence represented by SEQ ID NO: 3740.

The probes having the nucleotide sequence represented by SEQ ID NOS: 47346, 47347, 47348, 47349, 47350, 47351, 47352, 47353, 47354, 47355, 47356 were selected from the ORFs having the nucleotide sequence represented by SEQ ID NO: 3741.

The probes having the nucleotide sequence represented by SEQ ID NOS: 47357, 47358, 47359, 47360, 47361, 47362, 47363, 47364, 47365, 47366, 47367 were selected from the ORFs having the nucleotide sequence represented by SEQ ID NO: 3742.

The probes having the nucleotide sequence represented by SEQ ID NOS: 47368, 47369, 47370, 47371, 47372, 47373, 47374, 47375, 47376, 47377, 47378 were selected from the ORFs having the nucleotide sequence represented by SEQ ID NO: 3743.

The probes having the nucleotide sequence represented by SEQ ID NOS: 47379, 47380, 47381, 47382, 47383, 47384, 47385, 47386, 47387, 47388, 47389 were selected from the ORFs having the nucleotide sequence represented by SEQ ID NO: 3744.

The probes having the nucleotide sequence represented by SEQ ID NOS: 47390, 47391, 47392, 47393, 47394, 47395, 47396, 47397, 47398, 47399, 47400 were selected from the ORFs having the nucleotide sequence represented by SEQ ID NO: 3745.

The probes having the nucleotide sequence represented by SEQ ID NOS: 47401, 47402, 47403, 47404, 47405, 47406, 47407, 47408, 47409, 47410, 47411 were selected from the ORFs having the nucleotide sequence represented by SEQ ID NO: 3746.

The probes having the nucleotide sequence represented by SEQ ID NOS: 47412, 47413, 47414, 47415, 47416, 47417, 47418, 47419, 47420, 47421, 47422 were selected from the ORFs having the nucleotide sequence represented by SEQ ID NO: 3747.

The probes having the nucleotide sequence represented by SEQ ID NOS: 47423, 47424, 47425, 47426, 47427, 47428, 47429, 47430, 47431, 47432, 47433 were selected from the ORFs having the nucleotide sequence represented by SEQ ID NO: 3748.

The probes having the nucleotide sequence represented by SEQ ID NOS: 47434, 47435, 47436, 47437, 47438, 47439, 47440, 47441, 47442, 47443, 47444 were selected from the ORFs having the nucleotide sequence represented by SEQ ID NO: 3749.

The probes having the nucleotide sequence represented by SEQ ID NOS: 47445, 47446, 47447, 47448, 47449, 47450, 47451, 47452, 47453, 47454, 47455 were selected from the ORFs having the nucleotide sequence represented by SEQ ID NO: 3750.

The probes having the nucleotide sequence represented by SEQ ID NOS: 47456, 47457, 47458, 47459, 47460, 47461, 47462, 47463, 47464, 47465, 47466 were selected from the ORFs having the nucleotide sequence represented by SEQ ID NO: 3751.

The probes having the nucleotide sequence represented by SEQ ID NOS: 47467, 47468, 47469, 47470, 47471, 47472, 47473, 47474, 47475, 47476, 47477 were selected from the ORFs having the nucleotide sequence represented by SEQ ID NO: 3752.

The probes having the nucleotide sequence represented by SEQ ID NOS: 47478, 47479, 47480, 47481, 47482, 47483, 47484, 47485, 47486, 47487, 47488 were selected from the ORFs having the nucleotide sequence represented by SEQ ID NO: 3753.

The probes having the nucleotide sequence represented by SEQ ID NOS: 47489, 47490, 47491, 47492, 47493, 47494, 47495, 47496, 47497, 47498, 47499 were selected from the ORFs having the nucleotide sequence represented by SEQ ID NO: 3754.

The probes having the nucleotide sequence represented by SEQ ID NOS: 47500, 47501, 47502, 47503, 47504, 47505, 47506, 47507, 47508, 47509, 47510 were selected from the ORFs having the nucleotide sequence represented by SEQ ID NO: 3755.

The probes having the nucleotide sequence represented by SEQ ID NOS: 47511, 47512, 47513, 47514, 47515, 47516, 47517, 47518, 47519, 47520, 47521 were selected from the ORFs having the nucleotide sequence represented by SEQ ID NO: 3756.

The probes having the nucleotide sequence represented by SEQ ID NOS: 47522, 47523, 47524, 47525, 47526, 47527, 47528, 47529, 47530, 47531, 47532 were selected from the ORFs having the nucleotide sequence represented by SEQ ID NO: 3757.

The probes having the nucleotide sequence represented by SEQ ID NOS: 47533, 47534, 47535, 47536, 47537, 47538, 47539, 47540, 47541, 47542, 47543 were selected from the ORFs having the nucleotide sequence represented by SEQ ID NO: 3758.

The probes having the nucleotide sequence represented by SEQ ID NOS: 47544, 47545, 47546, 47547, 47548, 47549, 47550, 47551, 47552, 47553, 47554 were selected from the ORFs having the nucleotide sequence represented by SEQ ID NO: 3759.

The probes having the nucleotide sequence represented by SEQ ID NOS: 47555, 47556, 47557, 47558, 47559, 47560, 47561, 47562, 47563, 47564, 47565 were selected from the ORFs having the nucleotide sequence represented by SEQ ID NO: 3760.

The probes having the nucleotide sequence represented by SEQ ID NOS: 47566, 47567, 47568, 47569, 47570, 47571, 47572, 47573, 47574, 47575, 47576 were selected from the ORFs having the nucleotide sequence represented by SEQ ID NO: 3761.

The probes having the nucleotide sequence represented by SEQ ID NOS: 47577, 47578, 47579, 47580, 47581, 47582, 47583, 47584, 47585, 47586, 47587 were selected from the ORFs having the nucleotide sequence represented by SEQ ID NO: 3762.

The probes having the nucleotide sequence represented by SEQ ID NOS: 47588, 47589, 47590, 47591, 47592, 47593, 47594, 47595, 47596, 47597, 47598 were selected from the ORFs having the nucleotide sequence represented by SEQ ID NO: 3763.

The probes having the nucleotide sequence represented by SEQ ID NOS: 47599, 47600, 47601, 47602, 47603, 47604, 47605, 47606, 47607, 47608, 47609 were selected from the ORFs having the nucleotide sequence represented by SEQ ID NO: 3764.

The probes having the nucleotide sequence represented by SEQ ID NOS: 47610, 47611, 47612, 47613, 47614, 47615, 47616, 47617, 47618, 47619, 47620 were selected from the ORFs having the nucleotide sequence represented by SEQ ID NO: 3765.

The probes having the nucleotide sequence represented by SEQ ID NOS: 47621, 47622, 47623, 47624, 47625, 47626, 47627, 47628, 47629, 47630, 47631 were selected from the ORFs having the nucleotide sequence represented by SEQ ID NO: 3766.

The probes having the nucleotide sequence represented by SEQ ID NOS: 47632, 47633, 47634, 47635, 47636, 47637, 47638, 47639, 47640, 47641, 47642 were selected from the ORFs having the nucleotide sequence represented by SEQ ID NO: 3767.

The probes having the nucleotide sequence represented by SEQ ID NOS: 47643, 47644, 47645, 47646, 47647, 47648, 47649, 47650, 47651, 47652, 47653 were selected from the ORFs having the nucleotide sequence represented by SEQ ID NO: 3768.

The probes having the nucleotide sequence represented by SEQ ID NOS: 47654, 47655, 47656, 47657, 47658, 47659, 47660, 47661, 47662, 47663, 47664 were selected from the ORFs having the nucleotide sequence represented by SEQ ID NO: 3769.

The probes having the nucleotide sequence represented by SEQ ID NOS: 47665, 47666, 47667, 47668, 47669, 47670, 47671, 47672, 47673, 47674, 47675 were selected from the ORFs having the nucleotide sequence represented by SEQ ID NO: 3770.

The probes having the nucleotide sequence represented by SEQ ID NOS: 47676, 47677, 47678, 47679, 47680, 47681, 47682, 47683, 47684, 47685, 47686 were selected from the ORFs having the nucleotide sequence represented by SEQ ID NO: 3771.

The probes having the nucleotide sequence represented by SEQ ID NOS: 47687, 47688, 47689, 47690, 47691, 47692, 47693, 47694, 47695, 47696, 47697 were selected from the ORFs having the nucleotide sequence represented by SEQ ID NO: 3772.

The probes having the nucleotide sequence represented by SEQ ID NOS: 47698, 47699, 47700, 47701, 47702, 47703, 47704, 47705, 47706, 47707, 47708 were selected from the ORFs having the nucleotide sequence represented by SEQ ID NO: 3773.

The probes having the nucleotide sequence represented by SEQ ID NOS: 47709, 47710, 47711, 47712, 47713, 47714, 47715, 47716, 47717, 47718, 47719 were selected from the ORFs having the nucleotide sequence represented by SEQ ID NO: 3774.

The probes having the nucleotide sequence represented by SEQ ID NOS: 47720, 47721, 47722, 47723, 47724, 47725, 47726, 47727, 47728, 47729, 47730 were selected from the ORFs having the nucleotide sequence represented by SEQ ID NO: 3775.

The probes having the nucleotide sequence represented by SEQ ID NOS: 47731, 47732, 47733, 47734, 47735, 47736, 47737, 47738, 47739, 47740, 47741 were selected from the ORFs having the nucleotide sequence represented by SEQ ID NO: 3776.

The probes having the nucleotide sequence represented by SEQ ID NOS: 47742, 47743, 47744, 47745, 47746, 47747, 47748, 47749, 47750, 47751, 47752, 47753, 47754, 47755, 47756, 47757, 47758, 47759, 47760, 47761, 47762, 47763 were selected from the ORFs having the nucleotide sequence represented by SEQ ID NO: 3777.

The probes having the nucleotide sequence represented by SEQ ID NOS: 47764, 47765, 47766, 47767, 47768, 47769, 47770, 47771, 47772, 47773, 47774 were selected from the ORFs having the nucleotide sequence represented by SEQ ID NO: 3778.

The probes having the nucleotide sequence represented by SEQ ID NOS: 47775, 47776, 47777, 47778, 47779, 47780, 47781, 47782, 47783, 47784, 47785 were selected from the ORFs having the nucleotide sequence represented by SEQ ID NO: 3779.

The probes having the nucleotide sequence represented by SEQ ID NOS: 47786, 47787, 47788, 47789, 47790, 47791, 47792, 47793, 47794, 47795, 47796 were selected from the ORFs having the nucleotide sequence represented by SEQ ID NO: 3780.

The probes having the nucleotide sequence represented by SEQ ID NOS: 47797, 47798, 47799, 47800, 47801, 47802, 47803, 47804, 47805, 47806, 47807 were selected from the ORFs having the nucleotide sequence represented by SEQ ID NO: 3781.

The probes having the nucleotide sequence represented by SEQ ID NOS: 47808, 47809, 47810, 47811, 47812, 47813, 47814, 47815, 47816, 47817, 47818 were selected from the ORFs having the nucleotide sequence represented by SEQ ID NO: 3782.

The probes having the nucleotide sequence represented by SEQ ID NOS: 47819, 47820, 47821, 47822, 47823, 47824, 47825, 47826, 47827, 47828, 47829 were selected from the ORFs having the nucleotide sequence represented by SEQ ID NO: 3783.

The probes having the nucleotide sequence represented by SEQ ID NOS: 47830, 47831, 47832, 47833, 47834, 47835, 47836, 47837, 47838, 47839, 47840 were selected from the ORFs having the nucleotide sequence represented by SEQ ID NO: 3784.

The probes having the nucleotide sequence represented by SEQ ID NOS: 47841, 47842, 47843, 47844, 47845, 47846, 47847, 47848, 47849, 47850, 47851 were selected from the ORFs having the nucleotide sequence represented by SEQ ID NO: 3785.

The probes having the nucleotide sequence represented by SEQ ID NOS: 47852, 47853, 47854, 47855, 47856, 47857, 47858, 47859, 47860, 47861, 47862 were selected from the ORFs having the nucleotide sequence represented by SEQ ID NO: 3786.

The probes having the nucleotide sequence represented by SEQ ID NOS: 47863, 47864, 47865, 47866, 47867, 47868, 47869, 47870, 47871, 47872, 47873 were selected from the ORFs having the nucleotide sequence represented by SEQ ID NO: 3787.

The probes having the nucleotide sequence represented by SEQ ID NOS: 47874, 47875, 47876, 47877, 47878, 47879, 47880, 47881, 47882, 47883, 47884 were selected from the ORFs having the nucleotide sequence represented by SEQ ID NO: 3788.

The probes having the nucleotide sequence represented by SEQ ID NOS: 47885, 47886, 47887, 47888, 47889, 47890, 47891, 47892, 47893, 47894, 47895 were selected from the ORFs having the nucleotide sequence represented by SEQ ID NO: 3789.

The probes having the nucleotide sequence represented by SEQ ID NOS: 47896, 47897, 47898, 47899, 47900, 47901, 47902, 47903, 47904, 47905, 47906 were selected from the ORFs having the nucleotide sequence represented by SEQ ID NO: 3790.

The probes having the nucleotide sequence represented by SEQ ID NOS: 47907, 47908, 47909, 47910, 47911, 47912, 47913, 47914, 47915, 47916, 47917 were selected from the ORFs having the nucleotide sequence represented by SEQ ID NO: 3791.

The probes having the nucleotide sequence represented by SEQ ID NOS: 47918, 47919, 47920, 47921, 47922, 47923, 47924, 47925, 47926, 47927, 47928 were selected from the ORFs having the nucleotide sequence represented by SEQ ID NO: 3792.

The probes having the nucleotide sequence represented by SEQ ID NOS: 47929, 47930, 47931, 47932, 47933, 47934, 47935, 47936, 47937, 47938, 47939 were selected from the ORFs having the nucleotide sequence represented by SEQ ID NO: 3793.

The probes having the nucleotide sequence represented by SEQ ID NOS: 47940, 47941, 47942, 47943, 47944, 47945, 47946, 47947, 47948, 47949, 47950 were selected from the ORFs having the nucleotide sequence represented by SEQ ID NO: 3794.

The probes having the nucleotide sequence represented by SEQ ID NOS: 47951, 47952, 47953, 47954, 47955, 47956, 47957, 47958, 47959, 47960, 47961 were selected from the ORFs having the nucleotide sequence represented by SEQ ID NO: 3795.

The probes having the nucleotide sequence represented by SEQ ID NOS: 47962, 47963, 47964, 47965, 47966, 47967, 47968, 47969, 47970, 47971, 47972 were selected from the ORFs having the nucleotide sequence represented by SEQ ID NO: 3796.

The probes having the nucleotide sequence represented by SEQ ID NOS: 47973, 47974, 47975, 47976, 47977, 47978, 47979, 47980, 47981, 47982, 47983 were selected from the ORFs having the nucleotide sequence represented by SEQ ID NO: 3797.

The probes having the nucleotide sequence represented by SEQ ID NOS: 47984, 47985, 47986, 47987, 47988, 47989, 47990, 47991, 47992, 47993, 47994 were selected from the ORFs having the nucleotide sequence represented by SEQ ID NO: 3798.

The probes having the nucleotide sequence represented by SEQ ID NOS: 47995, 47996, 47997, 47998, 47999, 48000, 48001, 48002, 48003, 48004, 48005 were selected from the ORFs having the nucleotide sequence represented by SEQ ID NO: 3799.

The probes having the nucleotide sequence represented by SEQ ID NOS: 48006, 48007, 48008, 48009, 48010, 48011, 48012, 48013, 48014, 48015, 48016 were selected from the ORFs having the nucleotide sequence represented by SEQ ID NO: 3800.

The probes having the nucleotide sequence represented by SEQ ID NOS: 48017, 48018, 48019, 48020, 48021, 48022, 48023, 48024, 48025, 48026, 48027 were selected from the ORFs having the nucleotide sequence represented by SEQ ID NO: 3801.

The probes having the nucleotide sequence represented by SEQ ID NOS: 48028, 48029, 48030, 48031, 48032, 48033, 48034, 48035, 48036, 48037, 48038 were selected from the ORFs having the nucleotide sequence represented by SEQ ID NO: 3802.

The probes having the nucleotide sequence represented by SEQ ID NOS: 48039, 48040, 48041, 48042, 48043, 48044, 48045, 48046, 48047, 48048, 48049 were selected from the ORFs having the nucleotide sequence represented by SEQ ID NO: 3803.

The probes having the nucleotide sequence represented by SEQ ID NOS: 48050, 48051, 48052, 48053, 48054, 48055, 48056, 48057, 48058, 48059, 48060 were selected from the ORFs having the nucleotide sequence represented by SEQ ID NO: 3804.

The probes having the nucleotide sequence represented by SEQ ID NOS: 48061, 48062, 48063, 48064, 48065, 48066, 48067, 48068, 48069, 48070, 48071 were selected from the ORFs having the nucleotide sequence represented by SEQ ID NO: 3805.

The probes having the nucleotide sequence represented by SEQ ID NOS: 48072, 48073, 48074, 48075, 48076, 48077, 48078, 48079, 48080, 48081, 48082 were selected from the ORFs having the nucleotide sequence represented by SEQ ID NO: 3806.

The probes having the nucleotide sequence represented by SEQ ID NOS: 48083, 48084, 48085, 48086, 48087, 48088, 48089, 48090, 48091, 48092, 48093 were selected from the ORFs having the nucleotide sequence represented by SEQ ID NO: 3807.

The probes having the nucleotide sequence represented by SEQ ID NOS: 48094, 48095, 48096, 48097, 48098, 48099, 48100, 48101, 48102, 48103, 48104 were selected from the ORFs having the nucleotide sequence represented by SEQ ID NO: 3808.

The probes having the nucleotide sequence represented by SEQ ID NOS: 48105, 48106, 48107, 48108, 48109, 48110, 48111, 48112, 48113, 48114, 48115 were selected from the ORFs having the nucleotide sequence represented by SEQ ID NO: 3809.

The probes having the nucleotide sequence represented by SEQ ID NOS: 48116, 48117, 48118, 48119, 48120, 48121, 48122, 48123, 48124, 48125, 48126 were selected from the ORFs having the nucleotide sequence represented by SEQ ID NO: 3810.

The probes having the nucleotide sequence represented by SEQ ID NOS: 48127, 48128, 48129, 48130, 48131, 48132, 48133, 48134, 48135, 48136, 48137 were selected from the ORFs having the nucleotide sequence represented by SEQ ID NO: 3811.

The probes having the nucleotide sequence represented by SEQ ID NOS: 48138, 48139, 48140, 48141, 48142, 48143, 48144, 48145, 48146, 48147, 48148 were selected from the ORFs having the nucleotide sequence represented by SEQ ID NO: 3812.

The probes having the nucleotide sequence represented by SEQ ID NOS: 48149, 48150, 48151, 48152, 48153, 48154, 48155, 48156, 48157, 48158, 48159 were selected from the ORFs having the nucleotide sequence represented by SEQ ID NO: 3813.

The probes having the nucleotide sequence represented by SEQ ID NOS: 48160, 48161, 48162, 48163, 48164, 48165, 48166, 48167, 48168, 48169, 48170 were selected from the ORFs having the nucleotide sequence represented by SEQ ID NO: 3814.

The probes having the nucleotide sequence represented by SEQ ID NOS: 48171, 48172, 48173, 48174, 48175, 48176, 48177, 48178, 48179, 48180, 48181 were selected from the ORFs having the nucleotide sequence represented by SEQ ID NO: 3815.

The probes having the nucleotide sequence represented by SEQ ID NOS: 48182, 48183, 48184, 48185, 48186, 48187, 48188, 48189, 48190, 48191, 48192 were selected from the ORFs having the nucleotide sequence represented by SEQ ID NO: 3816.

The probes having the nucleotide sequence represented by SEQ ID NOS:. 48193, 48194, 48195, 48196, 48197, 48198, 48199, 48200, 48201, 48202, 48203 were selected from the ORFs having the nucleotide sequence represented by SEQ ID NO: 3817.

The probes having the nucleotide sequence represented by SEQ ID NOS: 48204, 48205, 48206, 48207, 48208, 48209, 48210, 48211, 48212, 48213, 48214 were selected from the ORFs having the nucleotide sequence represented by SEQ ID NO: 3818.

The probes having the nucleotide sequence represented by SEQ ID NOS: 48215, 48216, 48217, 48218, 48219, 48220, 48221, 48222, 48223, 48224, 48225 were selected from the ORFs having the nucleotide sequence represented by SEQ ID NO: 3819.

The probes having the nucleotide sequence represented by SEQ ID NOS: 48226, 48227, 48228, 48229, 48230, 48231, 48232, 48233, 48234, 48235, 48236 were selected from the ORFs having the nucleotide sequence represented by SEQ ID NO: 3820.

The probes having the nucleotide sequence represented by SEQ ID NOS: 48237, 48238, 48239, 48240, 48241, 48242, 48243, 48244, 48245, 48246, 48247 were selected from the ORFs having the nucleotide sequence represented by SEQ ID NO: 3821.

The probes having the nucleotide sequence represented by SEQ ID NOS: 48248, 48249, 48250, 48251, 48252, 48253, 48254, 48255, 48256, 48257, 48258 were selected from the ORFs having the nucleotide sequence represented by SEQ ID NO: 3822.

The probes having the nucleotide sequence represented by SEQ ID NOS: 48259, 48260, 48261, 48262, 48263, 48264, 48265, 48266, 48267, 48268, 48269 were selected from the ORFs having the nucleotide sequence represented by SEQ ID NO: 3823.

The probes having the nucleotide sequence represented by SEQ ID NOS: 48270, 48271, 48272, 48273, 48274, 48275, 48276, 48277, 48278, 48279, 48280 were selected from the ORFs having the nucleotide sequence represented by SEQ ID NO: 3824.

The probes having the nucleotide sequence represented by SEQ ID NOS: 48281, 48282, 48283, 48284, 48285, 48286, 48287, 48288, 48289, 48290, 48291 were selected from the ORFs having the nucleotide sequence represented by SEQ ID NO: 3825.

The probes having the nucleotide sequence represented by SEQ ID NOS: 48292, 48293, 48294, 48295, 48296, 48297, 48298, 48299, 48300, 48301, 48302 were selected from the ORFs having the nucleotide sequence represented by SEQ ID NO: 3826.

The probes having the nucleotide sequence represented by SEQ ID NOS: 48303, 48304, 48305, 48306, 48307, 48308, 48309, 48310, 48311, 48312, 48313, 48314, 48315, 48.316, 48317, 48318, 48319, 48320, 48321, 48322, 48323, 48324 were selected from the ORFs having the nucleotide sequence represented by SEQ ID NO: 3827.

The probes having the nucleotide sequence represented by SEQ ID NOS: 48325, 48326, 48327, 48328, 48329, 48330, 48331, 48332, 48333, 48334, 48335 were selected from the ORFs having the nucleotide sequence represented by SEQ ID NO: 3828.

The probes having the nucleotide sequence represented by SEQ ID NOS: 48336, 48337, 48338, 48339, 48340, 48341, 48342, 48343, 48344, 48345, 48346 were selected from the ORFs having the nucleotide sequence represented by SEQ ID NO: 3829.

The probes having the nucleotide sequence represented by SEQ ID NOS: 48347, 48348, 48349, 48350, 48351, 48352, 48353, 48354, 48355, 48356, 48357 were selected from the ORFs having the nucleotide sequence represented by SEQ ID NO: 3830.

The probes having the nucleotide sequence represented by SEQ ID NOS: 48358, 48359, 48360, 48361, 48362, 48363, 48364, 48365, 48366, 48367, 48368 were selected from the ORFs having the nucleotide sequence represented by SEQ ID NO: 3831.

The probes having the nucleotide sequence represented by SEQ ID NOS: 48369, 48370, 48371, 48372, 48373, 48374, 48375, 48376, 48377, 48378, 48379 were selected from the ORFs having the nucleotide sequence represented by SEQ ID NO: 3832.

The probes having the nucleotide sequence represented by SEQ ID NOS: 48380, 48381, 48382, 48383, 48384, 48385, 48386, 48387, 48388, 48389, 48390 were selected from the ORFs having the nucleotide sequence represented by SEQ ID NO: 3833.

The probes having the nucleotide sequence represented by SEQ ID NOS: 48391, 48392, 48393, 48394, 48395, 48396, 48397, 48398, 48399, 48400, 48401 were selected from the ORFs having the nucleotide sequence represented by SEQ ID NO: 3834.

The probes having the nucleotide sequence represented by SEQ ID NOS: 48402, 48403, 48404, 48405, 48406, 48407, 48408, 48409, 48410, 48411, 48412 were selected from the ORFs having the nucleotide sequence represented by SEQ ID NO: 3835.

The probes having the nucleotide sequence represented by SEQ ID NOS: 48413, 48414, 48415, 48416, 48417, 48418, 48419, 48420, 48421, 48422, 48423 were selected from the ORFs having the nucleotide sequence represented by SEQ ID NO: 3836.

The probes having the nucleotide sequence represented by SEQ ID NOS: 48424, 48425, 48426, 48427, 48428, 48429, 48430, 48431, 48432, 48433, 48434 were selected from the ORFs having the nucleotide sequence represented by SEQ ID NO: 3837.

The probes having the nucleotide sequence represented by SEQ ID NOS: 48435, 48436, 48437, 48438, 48439, 48440, 48441, 48442, 48443, 48444, 48445, 48446, 48447, 48448, 48449, 48450, 48451, 48452, 48453, 48454, 48455, 48456 were selected from the ORFs having the nucleotide sequence represented by SEQ ID NO: 3838.

The probes having the nucleotide sequence represented by SEQ ID NOS: 48457, 48458, 48459, 48460, 48461, 48462, 48463, 48464, 48465, 48466, 48467 were selected from the ORFs having the nucleotide sequence represented by SEQ ID NO: 3839.

The probes having the nucleotide sequence represented by SEQ ID NOS: 48468, 48469, 48470, 48471, 48472, 48473, 48474, 48475, 48476, 48477, 48478 were selected from the ORFs having the nucleotide sequence represented by SEQ ID NO: 3840.

The probes having the nucleotide sequence represented by SEQ ID NOS: 48479, 48480, 48481, 48482, 48483, 48484, 48485, 48486, 48487, 48488, 48489 were selected from the ORFs having the nucleotide sequence represented by SEQ ID NO: 3841.

The probes having the nucleotide sequence represented by SEQ ID NOS: 48490, 48491, 48492, 48493, 48494, 48495, 48496, 48497, 48498, 48499, 48500 were selected from the ORFs having the nucleotide sequence represented by SEQ ID NO: 3842.

The probes having the nucleotide sequence represented by SEQ ID NOS: 48501, 48502, 48503, 48504, 48505, 48506, 48507, 48508, 48509, 48510, 48511 were selected from the ORFs having the nucleotide sequence represented by SEQ ID NO: 3843.

The probes having the nucleotide sequence represented by SEQ ID NOS: 48512, 48513, 48514, 48515, 48516, 48517, 48518, 48519, 48520, 48521, 48522 were selected from the ORFs having the nucleotide sequence represented by SEQ ID NO: 3844.

The probes having the nucleotide sequence represented by SEQ ID NOS: 48523, 48524, 48525, 48526, 48527, 48528, 48529, 48530, 48531, 48532, 48533 were selected from the ORFs having the nucleotide sequence represented by SEQ ID NO: 3845.

The probes having the nucleotide sequence represented by SEQ ID NOS: 48534, 48535, 48536, 48537, 48538, 48539, 48540, 48541, 48542, 48543, 48544, 48545, 48546, 48547, 48548, 48549, 48550, 48551, 48552, 48553, 48554, 48555 were selected from the ORFs having the nucleotide sequence represented by SEQ ID NO: 3846.

The probes having the nucleotide sequence represented by SEQ ID NOS: 48556, 48557, 48558, 48559, 48560, 48561, 48562, 48563, 48564, 48565, 48566 were selected from the ORFs having the nucleotide sequence represented by SEQ ID NO: 3847.

The probes having the nucleotide sequence represented by SEQ ID NOS: 48567, 48568, 48569, 48570, 48571, 48572, 48573, 48574, 48575, 48576, 48577 were selected from the ORFs having the nucleotide sequence represented by SEQ ID NO: 3848.

The probes having the nucleotide sequence represented by SEQ ID NOS: 48578, 48579, 48580, 48581, 48582, 48583, 48584, 48585, 48586, 48587, 48588 were selected from the ORFs having the nucleotide sequence represented by SEQ ID NO: 3849.

The probes having the nucleotide sequence represented by SEQ ID NOS: 48589, 48590., 48591, 48592, 48593, 48594, 48595, 48596, 48597, 48598, 48599 were selected from the ORFs having the nucleotide sequence represented by SEQ ID NO: 3850.

The probes having the nucleotide sequence represented by SEQ ID NOS: 48600, 48601, 48602, 48603, 48604, 48605, 48606, 48607, 48608, 48609, 48610 were selected from the ORFs having the nucleotide sequence represented by SEQ ID NO: 3851.

The probes having the nucleotide sequence represented by SEQ ID NOS: 48611, 48612, 48613, 48614, 48615, 48616, 48617, 48618, 48619, 48620, 48621 were selected from the ORFs having the nucleotide sequence represented by SEQ ID NO: 3852.

The probes having the nucleotide sequence represented by SEQ ID NOS: 48622, 48623, 48624, 48625, 48626, 48627, 48628, 48629, 48630, 48631, 48632 were selected from the ORFs having the nucleotide sequence represented by SEQ ID NO: 3853.

The probes having the nucleotide sequence represented by SEQ ID NOS: 48633, 48634, 48635, 48636, 48637, 48638, 48639, 48640, 48641, 48642, 48643 were selected from the ORFs having the nucleotide sequence represented by SEQ ID NO: 3854.

The probes having the nucleotide sequence represented by SEQ ID NOS: 48644, 48645, 48646, 48647, 48648, 48649, 48650, 48651, 48652, 48653, 48654 were selected from the ORFs having the nucleotide sequence represented by SEQ ID NO: 3855.

The probes having the nucleotide sequence represented by SEQ ID NOS: 48655, 48656, 48657, 48658, 48659, 48660, 48661, 48662, 48663, 48664, 48665 were selected from the ORFs having the nucleotide sequence represented by SEQ ID NO: 3856.

The probes having the nucleotide sequence represented by SEQ ID NOS: 48666, 48667, 48668, 48669, 48670, 48671, 48672, 48673, 48674, 48675, 48676 were selected from the ORFs having the nucleotide sequence represented by SEQ ID NO: 3857.

The probes having the nucleotide sequence represented by SEQ ID NOS: 48677, 48678, 48679, 48680, 48681, 48682, 48683, 48684, 48685, 48686, 48687 were selected from the ORFs having the nucleotide sequence represented by SEQ ID NO: 3858.

The probes having the nucleotide sequence represented by SEQ ID NOS: 48688, 48689, 48690, 48691, 48692, 48693, 48694, 48695, 48696, 48697, 48698 were selected from the ORFs having the nucleotide sequence represented by SEQ ID NO: 3859.

The probes having the nucleotide sequence represented by SEQ ID NOS: 48699, 48700, 48701, 48702, 48703, 48704, 48705, 48706, 48707, 48708, 48709 were selected from the ORFs having the nucleotide sequence represented by SEQ ID NO: 3860.

The probes having the nucleotide sequence represented by SEQ ID NOS: 48710, 48711, 48712, 48713, 48714, 48715, 48716, 48717, 48718, 48719, 48720 were selected from the ORFs having the nucleotide sequence represented by SEQ ID NO: 3861.

The probes having the nucleotide sequence represented by SEQ ID NOS: 48721, 48722, 48723, 48724, 48725, 48726, 48727, 48728, 48729, 48730, 48731 were selected from the ORFs having the nucleotide sequence represented by SEQ ID NO: 3862.

The probes having the nucleotide sequence represented by SEQ ID NOS: 48732, 48733, 48734, 48735, 48736, 48737, 48738, 48739, 48740, 48741, 48742 were selected from the ORFs having the nucleotide sequence represented by SEQ ID NO: 3863.

The probes having the nucleotide sequence represented by SEQ ID NOS: 48743, 48744, 48745, 48746, 48747, 48748, 48749, 48750, 48751, 48752, 48753 were selected from the ORFs having the nucleotide sequence represented by SEQ ID NO: 3864.

The probes having the nucleotide sequence represented by SEQ ID NOS: 48754, 48755, 48756, 48757, 48758, 48759, 48760, 48761, 48762, 48763, 48764 were selected from the ORFs having the nucleotide sequence represented by SEQ ID NO: 3865.

The probes having the nucleotide sequence represented by SEQ ID NOS: 48765, 48766, 48767, 48768, 48769, 48770, 48771, 48772, 48773, 48774, 48775 were selected from the ORFs having the nucleotide sequence represented by SEQ ID NO: 3866.

The probes having the nucleotide sequence represented by SEQ ID NOS: 48776, 48777, 48778, 48779, 48780, 48781, 48782, 48783, 48784, 48785, 48786 were selected from the ORFs having the nucleotide sequence represented by SEQ ID NO: 3867.

The probes having the nucleotide sequence represented by SEQ ID NOS: 48787, 48788, 48789, 48790, 48791, 48792, 48793, 48794, 48795, 48796, 48797 were selected from the ORFs having the nucleotide sequence represented by SEQ ID NO: 3868.

The probes having the nucleotide sequence represented by SEQ ID NOS: 48798, 48799, 48800, 48801, 48802, 48803, 48804, 48805, 48806, 48807, 48808 were selected from the ORFs having the nucleotide sequence represented by SEQ ID NO: 3869.

The probes having the nucleotide sequence represented by SEQ ID NOS: 48809, 48810, 48811, 48812, 48813, 48814, 48815, 48816, 48817, 48818, 48819 were selected from the ORFs having the nucleotide sequence represented by SEQ ID NO: 3870.

The probes having the nucleotide sequence represented by SEQ ID NOS: 48820, 48821, 48822, 48823, 48824, 48825, 48826, 48827, 48828, 48829, 48830 were selected from the ORFs having the nucleotide sequence represented by SEQ ID NO: 3871.

The probes having the nucleotide sequence represented by SEQ ID NOS: 48831, 48832, 48833, 48834, 48835, 48836, 48837, 48838, 48839, 48840, 48841, 48842, 48843, 48844, 48845, 48846, 48847, 48848, 48849, 48850, 48851, 48852 were selected from the ORFs having the nucleotide sequence represented by SEQ ID NO: 3872.

The probes having the nucleotide sequence represented by SEQ ID NOS: 48853, 48854, 48855, 48856, 48857, 48858, 48859, 48860, 48861, 48862, 48863, 48864, 48865, 48866, 48867, 48868, 48869, 48870, 48871, 48872, 48873, 48874 were selected from the ORFs having the nucleotide sequence represented by SEQ ID NO: 3873.

The probes having the nucleotide sequence represented by SEQ ID NOS: 48875, 48876, 48877, 48878, 48879, 48880, 48881, 48882, 48883, 48884, 48885 were selected from the ORFs having the nucleotide sequence represented by SEQ ID NO: 3874.

The probes having the nucleotide sequence represented by SEQ ID NOS: 48886, 48887, 48888, 48889, 48890, 48891, 48892, 48893, 48894, 48895, 48896 were selected from the ORFs having the nucleotide sequence represented by SEQ ID NO: 3875.

The probes having the nucleotide sequence represented by SEQ ID NOS: 48897, 48898, 48899, 48900, 48901, 48902, 48903, 48904, 48905, 48906, 48907 were selected from the ORFs having the nucleotide sequence represented by SEQ ID NO: 3876.

The probes having the nucleotide sequence represented by SEQ ID NOS: 48908, 48909, 48910, 48911, 48912, 48913, 48914, 48915, 48916, 48917, 48918 were selected from the ORFs having the nucleotide sequence represented by SEQ ID NO: 3877.

The probes having the nucleotide sequence represented by SEQ ID NOS: 48919, 48920, 48921, 48922, 48923, 48924, 48925, 48926, 48927, 48928, 48929 were selected from the ORFs having the nucleotide sequence represented by SEQ ID NO: 3878.

The probes having the nucleotide sequence represented by SEQ ID NOS: 48930, 48931, 48932, 48933, 48934, 48935, 48936, 48937, 48938, 48939, 48940 were selected from the ORFs having the nucleotide sequence represented by SEQ ID NO: 3879.

The probes having the nucleotide sequence represented by SEQ ID NOS: 48941, 48942, 48943, 48944, 48945, 48946, 48947, 48948, 48949, 48950, 48951 were selected from the ORFs having the nucleotide sequence represented by SEQ ID NO: 3880.

The probes having the nucleotide sequence represented by SEQ ID NOS: 48952, 48953, 48954, 48955, 48956, 48957, 48958, 48959, 48960, 48961, 48962 were selected from the ORFs having the nucleotide sequence represented by SEQ ID NO: 3881.

The probes having the nucleotide sequence represented by SEQ ID NOS: 48963, 48964, 48965, 48966, 48967, 48968, 48969, 48970, 48971, 48972, 48973 were selected from the ORFs having the nucleotide sequence represented by SEQ ID NO: 3882.

The probes having the nucleotide sequence represented by SEQ ID NOS: 48974, 48975, 48976, 48977, 48978, 48979, 48980, 48981, 48982, 48983, 48984 were selected from the ORFs having the nucleotide sequence represented by SEQ ID NO: 3883.

The probes having the nucleotide sequence represented by SEQ ID NOS: 48985, 48986, 48987, 48988, 48989, 48990, 48991, 48992, 48993, 48994, 48995 were selected from the ORFs having the nucleotide sequence represented by SEQ ID NO: 3884.

The probes having the nucleotide sequence represented by SEQ ID NOS: 48996, 48997, 48998, 48999, 49000, 49001, 49002, 49003, 49004, 49005, 49006 were selected from the ORFs having the nucleotide sequence represented by SEQ ID NO: 3885.

The probes having the nucleotide sequence represented by SEQ ID NOS: 49007, 49008, 49009, 49010, 49011, 49012, 49013, 49014, 49015, 49016, 49017 were selected from the ORFs having the nucleotide sequence represented by SEQ ID NO: 3886.

The probes having the nucleotide sequence represented by SEQ ID NOS: 49018, 49019, 49020, 49021, 49022, 49023, 49024, 49025, 49026, 49027, 49028 were selected from the ORFs having the nucleotide sequence represented by SEQ ID NO: 3887.

The probes having the nucleotide sequence represented by SEQ ID NOS: 49029, 49030, 49031, 49032, 49033, 49034, 49035, 49036, 49037, 49038, 49039 were selected from the ORFs having the nucleotide sequence represented by SEQ ID NO: 3888.

The probes having the nucleotide sequence represented by SEQ ID NOS: 49040, 49041, 49042, 49043, 49044, 49045, 49046, 49047, 49048, 49049, 49050 were selected from the ORFs having the nucleotide sequence represented by SEQ ID NO: 3889.

The probes having the nucleotide sequence represented by SEQ ID NOS: 49051, 49052, 49053, 49054, 49055, 49056, 49057, 49058, 49059, 49060 were selected from the ORFs having the nucleotide sequence represented by SEQ ID NO: 3890.

The probes having the nucleotide sequence represented by SEQ ID NOS: 49061, 49062, 49063, 49064, 49065, 49066, 49067, 49068, 49069, 49070, 49071 were selected from the ORFs having the nucleotide sequence represented by SEQ ID NO: 3891.

The probes having the nucleotide sequence represented by SEQ ID NOS: 49072, 49073, 49074, 49075, 49076, 49077, 49078, 49079, 49080, 49081, 49082 were selected from the ORFs having the nucleotide sequence represented by SEQ ID NO: 3892.

The probes having the nucleotide sequence represented by SEQ ID NOS: 49083, 49084, 49085, 49086, 49087, 49088, 49089, 49090, 49091, 49092, 49093, 49094, 49095, 49096, 49097, 49098, 49099, 49100, 49101, 49102, 49103, 49104 were selected from the ORFs having the nucleotide sequence represented by SEQ ID NO: 3893.

The probes having the nucleotide sequence represented by SEQ ID NOS: 49105, 49106, 49107, 49108, 49109, 49110, 49111, 49112, 49113, 49114, 49115 were selected from the ORFs having the nucleotide sequence represented by SEQ ID NO: 3894.

The probes having the nucleotide sequence represented by SEQ ID NOS: 49116, 49117, 49118, 49119, 49120, 49121, 49122, 49123, 49124, 49125, 49126 were selected from the ORFs having the nucleotide sequence represented by SEQ ID NO: 3895.

The probes having the nucleotide sequence represented by SEQ ID NOS: 49127, 49128, 49129, 49130, 49131, 49132, 49133, 49134, 49135, 49136, 49137 were selected from the ORFs having the nucleotide sequence represented by SEQ ID NO: 3896.

The probes having the nucleotide sequence represented by SEQ ID NOS: 49138, 49139, 49140, 49141, 49142, 49143, 49144, 49145, 49146, 49147, 49148 were selected from the ORFs having the nucleotide sequence represented by SEQ ID NO: 3897.

The probes having the nucleotide sequence represented by SEQ ID NOS: 49149, 49150, 49151, 49152, 49153, 49154, 49155, 49156, 49157, 49158, 49159 were selected from the ORFs having the nucleotide sequence represented by SEQ ID NO: 3898.

The probes having the nucleotide sequence represented by SEQ ID NOS: 49160, 49161, 49162, 49163, 49164, 49165, 49166, 49167, 49168, 49169, 49170 were selected from the ORFs having the nucleotide sequence represented by SEQ ID NO: 3899.

The probes having the nucleotide sequence represented by SEQ ID NOS: 49171, 49172, 49173, 49174, 49175, 49176, 49177, 49178, 49179, 49180, 49181 were selected from the ORFs having the nucleotide sequence represented by SEQ ID NO: 3900.

The probes having the nucleotide sequence represented by SEQ ID NOS: 49182, 49183, 49184, 49185, 49186, 49187, 49188, 49189, 49190, 49191, 49192 were selected from the ORFs having the nucleotide sequence represented by SEQ ID NO: 3901.

The probes having the nucleotide sequence represented by SEQ ID NOS: 49193, 49194, 49195, 49196, 49197, 49198, 49199, 49200, 49201, 49202, 49203 were selected from the ORFs having the nucleotide sequence represented by SEQ ID NO: 3902.

The probes having the nucleotide sequence represented by SEQ ID NOS: 49204, 49205, 49206, 49207, 49208, 49209, 49210, 49211, 49212, 49213, 49214 were selected from the ORFs having the nucleotide sequence represented by SEQ ID NO: 3903.

The probes having the nucleotide sequence represented by SEQ ID NOS: 49215, 49216, 49217, 49218, 49219, 49220, 49221, 49222, 49223, 49224, 49225 were selected from the ORFs having the nucleotide sequence represented by SEQ ID NO: 3904.

The probes having the nucleotide sequence represented by SEQ ID NOS: 49226, 49227, 49228, 49229, 49230, 49231, 49232, 49233, 49234, 49235, 49236, 49237, 49238, 49239, 49240, 49241, 49242, 49243, 49244, 49245, 49246, 49247 were selected from the ORFs having the nucleotide sequence represented by SEQ ID NO: 3905.

The probes having the nucleotide sequence represented by SEQ ID NOS: 49248, 49249, 49250, 49251, 49252, 49253, 49254, 49255, 49256, 49257, 49258 were selected from the ORFs having the nucleotide sequence represented by SEQ ID NO: 3906.

The probes having the nucleotide sequence represented by SEQ ID NOS: 49259, 49260, 49261, 49262, 49263, 49264, 49265, 49266, 49267, 49268, 49269 were selected from the ORFs having the nucleotide sequence represented by SEQ ID NO: 3907.

The probes having the nucleotide sequence represented by SEQ ID NOS: 49270, 49271, 49272, 49273, 49274, 49275, 49276, 49277, 49278, 49279, 49280 were selected from the ORFs having the nucleotide sequence represented by SEQ ID NO: 3908.

The probes having the nucleotide sequence represented by SEQ ID NOS: 49281, 49282, 49283, 49284, 49285, 49286, 49287, 49288, 49289, 49290, 49291 were selected from the ORFs having the nucleotide sequence represented by SEQ ID NO: 3909.

The probes having the nucleotide sequence represented by SEQ ID NOS: 49292, 49293, 49294, 49295, 49296, 49297, 49298, 49299, 49300, 49301, 49302 were selected from the ORFs having the nucleotide sequence represented by SEQ ID NO: 3910.

The probes having the nucleotide sequence represented by SEQ ID NOS: 49303, 49304, 49305, 49306, 49307, 49308, 49309, 49310, 49311, 49312, 49313 were selected from the ORFs having the nucleotide sequence represented by SEQ ID NO: 3911.

The probes having the nucleotide sequence represented by SEQ ID NOS: 49314, 49315, 49316, 49317, 49318, 49319, 49320, 49321, 49322, 49323, 49324 were selected from the ORFs having the nucleotide sequence represented by SEQ ID NO: 3912.

The probes having the nucleotide sequence represented by SEQ ID NOS: 49325, 49326, 49327, 49328, 49329, 49330, 49331, 49332, 49333, 49334, 49335 were selected from the ORFs having the nucleotide sequence represented by SEQ ID NO: 3913.

The probes having the nucleotide sequence represented by SEQ ID NOS: 49336, 49337, 49338, 49339, 49340, 49341, 49342, 49343, 49344, 49345, 49346 were selected from the ORFs having the nucleotide sequence represented by SEQ ID NO: 3914.

The probes having the nucleotide sequence represented by SEQ ID NOS: 49347, 49348, 49349, 49350, 49351, 49352, 49353, 49354, 49355, 49356, 49357 were selected from the ORFs having the nucleotide sequence represented by SEQ ID NO: 3915.

The probes having the nucleotide sequence represented by SEQ ID NOS: 49358, 49359, 49360, 49361, 49362, 49363, 49364, 49365, 49366, 49367, 49368 were selected from the ORFs having the nucleotide sequence represented by SEQ ID NO: 3916.

The probes having the nucleotide sequence represented by SEQ ID NOS: 49369, 49370, 49371, 49372, 49373, 49374, 49375, 49376, 49377, 49378, 49379 were selected from the ORFs having the nucleotide sequence represented by SEQ ID NO: 3917.

The probes having the nucleotide sequence represented by SEQ ID NOS: 49380, 49381, 49382, 49383, 49384, 49385, 49386, 49387, 49388, 49389, 49390 were selected from the ORFs having the nucleotide sequence represented by SEQ ID NO: 3918.

The probes having the nucleotide sequence represented by SEQ ID NOS: 49391, 49392, 49393, 49394, 49395, 49396, 49397, 49398, 49399, 49400, 49401 were selected from the ORFs having the nucleotide sequence represented by SEQ ID NO: 3919.

The probes having the nucleotide sequence represented by SEQ ID NOS: 49402, 49403, 49404, 49405, 49406, 49407, 49408, 49409, 49410, 49411, 49412 were selected from the ORFs having the nucleotide sequence represented by SEQ ID NO: 3920.

The probes having the nucleotide sequence represented by SEQ ID NOS: 49413, 49414, 49415, 49416, 49417, 49418, 49419, 49420, 49421, 49422, 49423 were selected from the ORFs having the nucleotide sequence represented by SEQ ID NO: 3921.

The probes having the nucleotide sequence represented by SEQ ID NOS: 49424, 49425, 49426, 49427, 49428, 49429, 49430, 49431, 49432, 49433, 49434 were selected from the ORFs having the nucleotide sequence represented by SEQ ID NO: 3922.

The probes having the nucleotide sequence represented by SEQ ID NOS: 49435, 49436, 49437, 49438, 49439, 49440, 49441, 49442, 49443, 49444, 49445 were selected from the ORFs having the nucleotide sequence represented by SEQ ID NO: 3923.

The probes having the nucleotide sequence represented by SEQ ID NOS: 49446, 49447, 49448, 49449, 49450, 49451, 49452, 49453, 49454, 49455, 49456 were selected from the ORFs having the nucleotide sequence represented by SEQ ID NO: 3924.

The probes having the nucleotide sequence represented by SEQ ID NOS: 49457, 49458, 49459, 49460, 49461, 49462, 49463, 49464, 49465, 49466, 49467 were selected from the ORFs having the nucleotide sequence represented by SEQ ID NO: 3925.

The probes having the nucleotide sequence represented by SEQ ID NOS: 49468, 49469, 49470, 49471, 49472, 49473, 49474, 49475, 49476, 49477, 49478 were selected from the ORFs having the nucleotide sequence represented by SEQ ID NO: 3926.

The probes having the nucleotide sequence represented by SEQ ID NOS: 49479, 49480, 49481, 49482, 49483, 49484, 49485, 49486, 49487, 49488, 49489 were selected from the ORFs having the nucleotide sequence represented by SEQ ID NO: 3927.

The probes having the nucleotide sequence represented by SEQ ID NOS: 49490, 49491, 49492, 49493, 49494, 49495, 49496, 49497, 49498, 49499, 49500 were selected from the ORFs having the nucleotide sequence represented by SEQ ID NO: 3928.

The probes having the nucleotide sequence represented by SEQ ID NOS: 49501, 49502, 49503, 49504, 49505, 49506, 49507, 49508, 49509, 49510, 49511 were selected from the ORFs having the nucleotide sequence represented by SEQ ID NO: 3929.

The probes having the nucleotide sequence represented by SEQ ID NOS: 49512, 49513, 49514, 49515, 49516, 49517, 49518, 49519, 49520, 49521, 49522 were selected from the ORFs having the nucleotide sequence represented by SEQ ID NO: 3930.

The probes having the nucleotide sequence represented by SEQ ID NOS: 49523, 49524, 49525, 49526, 49527, 49528, 49529, 49530, 49531, 49532, 49533 were selected from the ORFs having the nucleotide sequence represented by SEQ ID NO: 3931.

The probes having the nucleotide sequence represented by SEQ ID NOS: 49534, 49535, 49536, 49537, 49538, 49539, 49540, 49541, 49542, 49543, 49544 were selected from the ORFs having the nucleotide sequence represented by SEQ ID NO: 3932.

The probes having the nucleotide sequence represented by SEQ ID NOS: 49545, 49546, 49547, 49548, 49549, 49550, 49551, 49552, 49553, 49554, 49555 were selected from the ORFs having the nucleotide sequence represented by SEQ ID NO: 3933.

The probes having the nucleotide sequence represented by SEQ ID NOS: 49556, 49557, 49558, 49559, 49560, 49561, 49562, 49563, 49564, 49565, 49566 were selected from the ORFs having the nucleotide sequence represented by SEQ ID NO: 3934.

The probes having the nucleotide sequence represented by SEQ ID NOS: 49567, 49568, 49569, 49570, 49571, 49572, 49573, 49574, 49575, 49576, 49577 were selected from the ORFs having the nucleotide sequence represented by SEQ ID NO: 3935.

The probes having the nucleotide sequence represented by SEQ ID NOS: 49578, 49579, 49580, 49581, 49582, 49583, 49584, 49585, 49586, 49587, 49588 were selected from the ORFs having the nucleotide sequence represented by SEQ ID NO: 3936.

The probes having the nucleotide sequence represented by SEQ ID NOS: 49589, 49590, 49591, 49592, 49593, 49594, 49595, 49596, 49597, 49598, 49599 were selected from the ORFs having the nucleotide sequence represented by SEQ ID NO: 3937.

The probes having the nucleotide sequence represented by SEQ ID NOS: 49600, 49601, 49602, 49603, 49604, 49605, 49606, 49607, 49608, 49609, 49610 were selected from the ORFs having the nucleotide sequence represented by SEQ ID NO: 3938.

The probes having the nucleotide sequence represented by SEQ ID NOS: 49611, 49612, 49613, 49614, 49615, 49616, 49617, 49618, 49619, 49620, 49621 were selected from the ORFs having the nucleotide sequence represented by SEQ ID NO: 3939.

The probes having the nucleotide sequence represented by SEQ ID NOS: 49622, 49623, 49624, 49625, 49626, 49627, 49628, 49629, 49630, 49631, 49632 were selected from the ORFs having the nucleotide sequence represented by SEQ ID NO: 3940.

The probes having the nucleotide sequence represented by SEQ ID NOS: 49633, 49634, 49635, 49636, 49637, 49638, 49639, 49640, 49641, 49642, 49643 were selected from the ORFs having the nucleotide sequence represented by SEQ ID NO: 3941.

The probes having the nucleotide sequence represented by SEQ ID NOS: 49644, 49645, 49646, 49647, 49648, 49649, 49650, 49651, 49652, 49653, 49654 were selected from the ORFs having the nucleotide sequence represented by SEQ ID NO: 3942.

The probes having the nucleotide sequence represented by SEQ ID NOS: 49655, 49656, 49657, 49658, 49659, 49660, 49661, 49662, 49663, 49664, 49665 were selected from the ORFs having the nucleotide sequence represented by SEQ ID NO: 3943.

The probes having the nucleotide sequence represented by SEQ ID NOS: 49666, 49667, 49668, 49669, 49670, 49671, 49672, 49673, 49674, 49675, 49676 were selected from the ORFs having the nucleotide sequence represented by SEQ ID NO: 3944.

The probes having the nucleotide sequence represented by SEQ ID NOS: 49677, 49678, 49679, 49680, 49681, 49682, 49683, 49684, 49685, 49686, 49687 were selected from the ORFs having the nucleotide sequence represented by SEQ ID NO: 3945.

The probes having the nucleotide sequence represented by SEQ ID NOS: 49688, 49689, 49690, 49691, 49692, 49693, 49694, 49695, 49696, 49697, 49698 were selected from the ORFs having the nucleotide sequence represented by SEQ ID NO: 3946.

The probes having the nucleotide sequence represented by SEQ ID NOS: 49699, 49700, 49701, 49702, 49703, 49704, 49705, 49706, 49707, 49708, 49709 were selected from the ORFs having the nucleotide sequence represented by SEQ ID NO: 3947.

The probes having the nucleotide sequence represented by SEQ ID NOS: 49710, 49711, 49712, 49713, 49714, 49715, 49716, 49717, 49718, 49719, 49720 were selected from the ORFs having the nucleotide sequence represented by SEQ ID NO: 3948.

The probes having the nucleotide sequence represented by SEQ ID NOS: 49721, 49722, 49723, 49724, 49725, 49726, 49727, 49728, 49729, 49730, 49731 were selected from the ORFs having the nucleotide sequence represented by SEQ ID NO: 3949.

The probes having the nucleotide sequence represented by SEQ ID NOS: 49732, 49733, 49734, 49735, 49736, 49737, 49738, 49739, 49740, 49741, 49742 were selected from the ORFs having the nucleotide sequence represented by SEQ ID NO: 3950.

The probes having the nucleotide sequence represented by SEQ ID NOS: 49743, 49744, 49745, 49746, 49747, 49748, 49749, 49750, 49751, 49752, 49753 were selected from the ORFs having the nucleotide sequence represented by SEQ ID NO: 3951.

The probes having the nucleotide sequence represented by SEQ ID NOS: 49754, 49755, 49756, 49757, 49758, 49759, 49760, 49761, 49762, 49763, 49764 were selected from the ORFs having the nucleotide sequence represented by SEQ ID NO: 3952.

The probes having the nucleotide sequence represented by SEQ ID NOS: 49765, 49766, 49767, 49768, 49769, 49770, 49771, 49772, 49773, 49774, 49775 were selected from the ORFs having the nucleotide sequence represented by SEQ ID NO: 3953.

The probes having the nucleotide sequence represented by SEQ ID NOS: 49776, 49777, 49778, 49779, 49780, 49781, 49782, 49783, 49784, 49785, 49786 were selected from the ORFs having the nucleotide sequence represented by SEQ ID NO: 3954.

The probes having the nucleotide sequence represented by SEQ ID NOS: 49787, 49788, 49789, 49790, 49791, 49792, 49793, 49794, 49795, 49796, 49797 were selected from the ORFs having the nucleotide sequence represented by SEQ ID NO: 3955.

The probes having the nucleotide sequence represented by SEQ ID NOS: 49798, 49799, 49800, 49801, 49802, 49803, 49804, 49805, 49806, 49807, 49808 were selected from the ORFs having the nucleotide sequence represented by SEQ ID NO: 3956.

The probes having the nucleotide sequence represented by SEQ ID NOS: 49809, 49810, 49811, 49812, 49813, 49814, 49815, 49816, 49817, 49818, 49819 were selected from the ORFs having the nucleotide sequence represented by SEQ ID NO: 3957.

The probes having the nucleotide sequence represented by SEQ ID NOS: 49820, 49821, 49822, 49823, 49824, 49825, 49826, 49827, 49828, 49829, 49830 were selected from the ORFs having the nucleotide sequence represented by SEQ ID NO: 3958.

The probes having the nucleotide sequence represented by SEQ ID NOS: 49831, 49832, 49833, 49834, 49835, 49836, 49837, 49838, 49839, 49840, 49841 were selected from the ORFs having the nucleotide sequence represented by SEQ ID NO: 3959.

The probes having the nucleotide sequence represented by SEQ ID NOS: 49842, 49843, 49844, 49845, 49846, 49847, 49848, 49849, 49850, 49851, 49852 were selected from the ORFs having the nucleotide sequence represented by SEQ ID NO: 3960.

The probes having the nucleotide sequence represented by SEQ ID NOS: 49853, 49854, 49855, 49856, 49857, 49858, 49859, 49860, 49861, 49862, 49863, 49864, 49865, 49866, 49867, 49868, 49869, 49870, 49871, 49872, 49873, 49874 were selected from the ORFs having the nucleotide sequence represented by SEQ ID NO: 3961.

The probes having the nucleotide sequence represented by SEQ ID NOS: 49875, 49876, 49877, 49878, 49879, 49880, 49881, 49882, 49883, 49884, 49885 were selected from the ORFs having the nucleotide sequence represented by SEQ ID NO: 3962.

The probes having the nucleotide sequence represented by SEQ ID NOS: 49886, 49887, 49888, 49889, 49890, 49891, 49892, 49893, 49894, 49895, 49896 were selected from the ORFs having the nucleotide sequence represented by SEQ ID NO: 3963.

The probes having the nucleotide sequence represented by SEQ ID NOS: 49897, 49898, 49899, 49900, 49901, 49902, 49903, 49904, 49905, 49906, 49907 were selected from the ORFs having the nucleotide sequence represented by SEQ ID NO: 3964.

The probes having the nucleotide sequence represented by SEQ ID NOS: 49908, 49909, 49910, 49911, 49912, 49913, 49914, 49915, 49916, 49917, 49918 were selected from the ORFs having the nucleotide sequence represented by SEQ ID NO: 3965.

The probes having the nucleotide sequence represented by SEQ ID NOS: 49919, 49920, 49921, 49922, 49923, 49924, 49925, 49926, 49927, 49928, 49929 were selected from the ORFs having the nucleotide sequence represented by SEQ ID NO: 3966.

The probes having the nucleotide sequence represented by SEQ ID NOS: 49930, 49931, 49932, 49933, 49934, 49935, 49936, 49937, 49938, 49939, 49940 were selected from the ORFs having the nucleotide sequence represented by SEQ ID NO: 3967.

The probes having the nucleotide sequence represented by SEQ ID NOS: 49941, 49942, 49943, 49944, 49945, 49946, 49947, 49948, 49949, 49950, 49951 were selected from the ORFs having the nucleotide sequence represented by SEQ ID NO: 3968.

The probes having the nucleotide sequence represented by SEQ ID NOS: 49952, 49953, 49954, 49955, 49956, 49957, 49958, 49959, 49960, 49961, 49962 were selected from the ORFs having the nucleotide sequence represented by SEQ ID NO: 3969.

The probes having the nucleotide sequence represented by SEQ ID NOS: 49963, 49964, 49965, 49966, 49967, 49968, 49969, 49970, 49971, 49972, 49973 were selected from the ORFs having the nucleotide sequence represented by SEQ ID NO: 3970.

The probes having the nucleotide sequence represented by SEQ ID NOS: 49974, 49975, 49976, 49977, 49978, 49979, 49980, 49981, 49982, 49983, 49984 were selected from the ORFs having the nucleotide sequence represented by SEQ ID NO: 3971.

The probes having the nucleotide sequence represented by SEQ ID NOS: 49985, 49986, 49987, 49988, 49989, 49990, 49991, 49992, 49993, 49994, 49995 were selected from the ORFs having the nucleotide sequence represented by SEQ ID NO: 3972.

The probes having the nucleotide sequence represented by SEQ ID NOS: 49996, 49997, 49998, 49999, 50000, 50001, 50002, 50003, 50004, 50005, 50006 were selected from the ORFs having the nucleotide sequence represented by SEQ ID NO: 3973.

The probes having the nucleotide sequence represented by SEQ ID NOS: 50007, 50008, 50009, 50010, 50011, 50012, 50013, 50014, 50015, 50016, 50017 were selected from the ORFs having the nucleotide sequence represented by SEQ ID NO: 3974.

The probes having the nucleotide sequence represented by SEQ ID NOS: 50018, 50019, 50020, 50021, 50022, 50023, 50024, 50025, 50026, 50027, 50028 were selected from the ORFs having the nucleotide sequence represented by SEQ ID NO: 3975.

The probes having the nucleotide sequence represented by SEQ ID NOS: 50029, 50030, 50031, 50032, 50033, 50034, 50035, 50036, 50037, 50038, 50039 were selected from the ORFs having the nucleotide sequence represented by SEQ ID NO: 3976.

The probes having the nucleotide sequence represented by SEQ ID NOS: 50040, 50041, 50042, 50043, 50044, 50045, 50046, 50047, 50048, 50049, 50050 were selected from the ORFs having the nucleotide sequence represented by SEQ ID NO: 3977.

The probes having the nucleotide sequence represented by SEQ ID NOS: 50051, 50052, 50053, 50054, 50055, 50056, 50057, 50058, 50059, 50060, 50061 were selected from the ORFs having the nucleotide sequence represented by SEQ ID NO: 3978.

The probes having the nucleotide sequence represented by SEQ ID NOS: 50062, 50063, 50064, 50065, 50066, 50067, 50068, 50069, 50070, 50071, 50072 were selected from the ORFs having the nucleotide sequence represented by SEQ ID NO: 3979.

The probes having the nucleotide sequence represented by SEQ ID NOS: 50073, 50074, 50075, 50076, 50077, 50078, 50079, 50080, 50081, 50082, 50083 were selected from the ORFs having the nucleotide sequence represented by SEQ ID NO: 3980.

The probes having the nucleotide sequence represented by SEQ ID NOS: 50084, 50085, 50086, 50087, 50088, 50089, 50090, 50091, 50092, 50093, 50094 were selected from the ORFs having the nucleotide sequence represented by SEQ ID NO: 3981.

The probes having the nucleotide sequence represented by SEQ ID NOS: 50095, 50096, 50097, 50098, 50099, 50100, 50101, 50102, 50103, 50104, 50105 were selected from the ORFs having the nucleotide sequence represented by SEQ ID NO: 3982.

The probes having the nucleotide sequence represented by SEQ ID NOS: 50106, 50107, 50108, 50109, 50110, 50111, 50112, 50113, 50114, 50115, 50116 were selected from the ORFs having the nucleotide sequence represented by SEQ ID NO: 3983.

The probes having the nucleotide sequence represented by SEQ ID NOS: 50117, 50118, 50119, 50120, 50121, 50122, 50123, 50124, 50125, 50126, 50127 were selected from the ORFs having the nucleotide sequence represented by SEQ ID NO: 3984.

The probes having the nucleotide sequence represented by SEQ ID NOS: 50128, 50129, 50130, 50131, 50132, 50133, 50134, 50135, 50136, 50137, 50138 were selected from the ORFs having the nucleotide sequence represented by SEQ ID NO: 3985.

The probes having the nucleotide sequence represented by SEQ ID NOS: 50139, 50140, 50141, 50142, 50143, 50144, 50145, 50146, 50147, 50148, 50149 were selected from the ORFs having the nucleotide sequence represented by SEQ ID NO: 3986.

The probes having the nucleotide sequence represented by SEQ ID NOS: 50150, 50151, 50152, 50153, 50154, 50155, 50156, 50157, 50158, 50159, 50160 were selected from the ORFs having the nucleotide sequence represented by SEQ ID NO: 3987.

The probes having the nucleotide sequence represented by SEQ ID NOS: 50161, 50162, 50163, 50164, 50165, 50166, 50167, 50168, 50169, 50170, 50171 were selected from the ORFs having the nucleotide sequence represented by SEQ ID NO: 3988.

The probes having the nucleotide sequence represented by SEQ ID NOS: 50172, 50173, 50174, 50175, 50176, 50177, 50178, 50179, 50180, 50181, 50182 were selected from the ORFs having the nucleotide sequence represented by SEQ ID NO: 3989.

The probes having the nucleotide sequence represented by SEQ ID NOS: 50183, 50184, 50185, 50186, 50187, 50188, 50189, 50190, 50191, 50192, 50193 were selected from the ORFs having the nucleotide sequence represented by SEQ ID NO: 3990.

The probes having the nucleotide sequence represented by SEQ ID NOS: 50194, 50195, 50196, 50197, 50198, 50199, 50200, 50201, 50202, 50203, 50204 were selected from the ORFs having the nucleotide sequence represented by SEQ ID NO: 3991.

The probes having the nucleotide sequence represented by SEQ ID NOS: 50205, 50206, 50207, 50208, 50209, 50210, 50211, 50212, 50213, 50214, 50215 were selected from the ORFs having the nucleotide sequence represented by SEQ ID NO: 3992.

The probes having the nucleotide sequence represented by SEQ ID NOS: 50216, 50217, 50218, 50219, 50220, 50221, 50222, 50223, 50224, 50225, 50226 were selected from the ORFs having the nucleotide sequence represented by SEQ ID NO: 3993.

The probes having the nucleotide sequence represented by SEQ ID NOS: 50227, 50228, 50229, 50230, 50231, 50232, 50233, 50234, 50235, 50236, 50237 were selected from the ORFs having the nucleotide sequence represented by SEQ ID NO: 3994.

The probes having the nucleotide sequence represented by SEQ ID NOS: 50238, 50239, 50240, 50241, 50242, 50243, 50244, 50245, 50246, 50247, 50248 were selected from the ORFs having the nucleotide sequence represented by SEQ ID NO: 3995.

The probes having the nucleotide sequence represented by SEQ ID NOS: 50249, 50250, 50251, 50252, 50253, 50254, 50255, 50256, 50257, 50258, 50259 were selected from the ORFs having the nucleotide sequence represented by SEQ ID NO: 3996.

The probes having the nucleotide sequence represented by SEQ ID NOS: 50260, 50261, 50262, 50263, 50264, 50265, 50266, 50267, 50268, 50269, 50270 were selected from the ORFs having the nucleotide sequence represented by SEQ ID NO: 3997.

The probes having the nucleotide sequence represented by SEQ ID NOS: 50271, 50272, 50273, 50274, 50275, 50276, 50277, 50278, 50279, 50280., 50281 were selected from the ORFs having the nucleotide sequence represented by SEQ ID NO: 3998.

The probes having the nucleotide sequence represented by SEQ ID NOS: 50282, 50283, 50284, 50285, 50286, 50287, 50288, 50289, 50290, 50291, 50292 were selected from the ORFs having the nucleotide sequence represented by SEQ ID NO: 3999.

The probes having the nucleotide sequence represented by SEQ ID NOS: 50293, 50294, 50295, 50296, 50297, 50298, 50299, 50300, 50301, 50302, 50303 were selected from the ORFs having the nucleotide sequence represented by SEQ ID NO: 4000.

The probes having the nucleotide sequence represented by SEQ ID NOS: 50304, 50305, 50306, 50307, 50308, 50309, 50310, 50311, 50312, 50313, 50314 were selected from the ORFs having the nucleotide sequence represented by SEQ ID NO: 4001.

The probes having the nucleotide sequence represented by SEQ ID NOS: 50315, 50316, 50317, 50318, 50319, 50320, 50321, 50322, 50323, 50324, 50325 were selected from the ORFs having the nucleotide sequence represented by SEQ ID NO: 4002.

The probes having the nucleotide sequence represented by SEQ ID NOS: 50326, 50327, 50328, 50329, 50330, 50331, 50332, 50333, 50334, 50335, 50336 were selected from the ORFs having the nucleotide sequence represented by SEQ ID NO: 4003.

The probes having the nucleotide sequence represented by SEQ ID NOS: 50337, 50338, 50339, 50340, 50341, 50342, 50343, 50344, 50345, 50346, 50347 were selected from the ORFs having the nucleotide sequence represented by SEQ ID NO: 4004.

The probes having the nucleotide sequence represented by SEQ ID NOS: 50348, 50349, 50350, 50351, 50352, 50353, 50354, 50355, 50356, 50357, 50358 were selected from the ORFs having the nucleotide sequence represented by SEQ ID NO: 4005.

The probes having the nucleotide sequence represented by SEQ ID NOS: 50359, 50360, 50361, 50362, 50363, 50364, 50365, 50366, 50367, 50368, 50369 were selected from the ORFs having the nucleotide sequence represented by SEQ ID NO: 4006.

The probes having the nucleotide sequence represented by SEQ ID NOS: 50370, 50371, 50372, 50373, 50374, 50375, 50376, 50377, 50378, 50379, 50380 were selected from the ORFs having the nucleotide sequence represented by SEQ ID NO: 4007.

The probes having the nucleotide sequence represented by SEQ ID NOS: 50381, 50382, 50383, 50384, 50385, 50386, 50387, 50388, 50389, 50390, 50391 were selected from the ORFs having the nucleotide sequence represented by SEQ ID NO: 4008.

The probes having the nucleotide sequence represented by SEQ ID NOS: 50392, 50393, 50394, 50395, 50396, 50397, 50398, 50399, 50400, 50401, 50402 were selected from the ORFs having the nucleotide sequence represented by SEQ ID NO: 4009.

The probes having the nucleotide sequence represented by SEQ ID NOS: 50403, 50404, 50405, 50406, 50407, 50408, 50409, 50410, 50411, 50412, 50413 were selected from the ORFs having the nucleotide sequence represented by SEQ ID NO: 4010.

The probes having the nucleotide sequence represented by SEQ ID NOS: 50414, 50415, 50416, 50417, 50418, 50419, 50420, 50421, 50422, 50423, 50424 were selected from the ORFs having the nucleotide sequence represented by SEQ ID NO: 4011.

The probes having the nucleotide sequence represented by SEQ ID NOS: 50425, 50426, 50427, 50428, 50429, 50430, 50431, 50432, 50433, 50434, 50435 were selected from the ORFs having the nucleotide sequence represented by SEQ ID NO: 4012.

The probes having the nucleotide sequence represented by SEQ ID NOS: 50436, 50437, 50438, 50439, 50440, 50441, 50442, 50443, 50444, 50445, 50446 were selected from the ORFs having the nucleotide sequence represented by SEQ ID NO: 4013.

The probes having the nucleotide sequence represented by SEQ ID NOS: 50447, 50448, 50449, 50450, 50451, 50452, 50453, 50454, 50455, 50456, 50457 were selected from the ORFs having the nucleotide sequence represented by SEQ ID NO: 4014.

The probes having the nucleotide sequence represented by SEQ ID NOS: 50458, 50459, 50460, 50461, 50462, 50463, 50464, 50465, 50466, 50467, 50468 were selected from the ORFs having the nucleotide sequence represented by SEQ ID NO: 4015.

The probes having the nucleotide sequence represented by SEQ ID NOS: 50502, 50503, 50504, 50505, 50506, 50507, 50508, 50509, 50510, 50511, 50512, 50513, 50514, 50515, 50516, 50517, 50518, 50519, 50520, 50521, 50522, 50523 were selected from the ORFs having the nucleotide sequence represented by SEQ ID NO: 4016.

The probes having the nucleotide sequence represented by SEQ ID NOS: 50469, 50470, 50471, 50472, 50473, 50474, 50475, 50476, 50477, 50478, 50479 were selected from the ORFs having the nucleotide sequence represented by SEQ ID NO: 4017.

The probes having the nucleotide sequence represented by SEQ ID NOS: 50480, 50481, 50482, 50483, 50484, 50485, 50486, 50487, 50488, 50489, 50490, 50491, 50492, 50493, 50494, 50495, 50496, 50497, 50498, 50499, 50500, 50501 were selected from the ORFs having the nucleotide sequence represented by SEQ ID NO: 4018.

The probes having the nucleotide sequence represented by SEQ ID NOS: 50524, 50525, 50526, 50527, 50528, 50529, 50530, 50531, 50532, 50533, 50534 were selected from the ORFs having the nucleotide sequence represented by SEQ ID NO: 4019.

The probes having the nucleotide sequence represented by SEQ ID NOS: 50535, 50536, 50537, 50538, 50539, 50540, 50541, 50542, 50543, 50544, 50545 were selected from the ORFs having the nucleotide sequence represented by SEQ ID NO: 4020.

The probes having the nucleotide sequence represented by SEQ ID NOS: 50546, 50547, 50548, 50549, 50550, 50551, 50552, 50553, 50554, 50555, 50556 were selected from the ORFs having the nucleotide sequence represented by SEQ ID NO: 4021.

The probes having the nucleotide sequence represented by SEQ ID NOS: 50557, 50558, 50559, 50560, 50561, 50562, 50563, 50564, 50565, 50566, 50567 were selected from the ORFs having the nucleotide sequence represented by SEQ ID NO: 4022.

The probes having the nucleotide sequence represented by SEQ ID NOS: 50568, 50569, 50570, 50571, 50572, 50573, 50574, 50575, 50576, 50577, 50578 were selected from the ORFs having the nucleotide sequence represented by SEQ ID NO: 4023.

The probes having the nucleotide sequence represented by SEQ ID NOS: 50579, 50580, 50581, 50582, 50583, 50584, 50585, 50586, 50587, 50588, 50589 were selected from the ORFs having the nucleotide sequence represented by SEQ ID NO: 4024.

The probes having the nucleotide sequence represented by SEQ ID NOS: 50590, 50591, 50592, 50593, 50594, 50595, 50596, 50597, 50598, 50599, 50600 were selected from the ORFs having the nucleotide sequence represented by SEQ ID NO: 4025.

The probes having the nucleotide sequence represented by SEQ ID NOS: 50601, 50602, 50603, 50604, 50605, 50606, 50607, 50608, 50609, 50610, 50611 were selected from the ORFs having the nucleotide sequence represented by SEQ ID NO: 4026.

The probes having the nucleotide sequence represented by SEQ ID NOS:,50612, 50613, 50614, 50615, 50616, 50617, 50618, 50619, 50620, 50621, 50622 were selected from the ORFs having the nucleotide sequence represented by SEQ ID NO: 4027.

The probes having the nucleotide sequence represented by SEQ ID NOS: 50623, 50624, 50625, 50626, 50627, 50628, 50629, 50630, 50631, 50632, 50633 were selected from the ORFs having the nucleotide sequence represented by SEQ ID NO: 4028.

The probes having the nucleotide sequence represented by SEQ ID NOS: 50634, 50635, 50636, 50637, 50638, 50639, 50640, 50641, 50642, 50643, 50644 were selected from the ORFs having the nucleotide sequence represented by SEQ ID NO: 4029.

The probes having the nucleotide sequence represented by SEQ ID NOS: 50645, 50646, 50647, 50648, 50649, 50650, 50651, 50652, 50653, 50654, 50655 were selected from the ORFs having the nucleotide sequence represented by SEQ ID NO: 4030.

The probes having the nucleotide sequence represented by SEQ ID NOS: 50656, 50657, 50658, 50659, 50660, 50661, 50662, 50663, 50664, 50665, 50666 were selected from the ORFs having the nucleotide sequence represented by SEQ ID NO: 4031.

The probes having the nucleotide sequence represented by SEQ ID NOS: 50667, 50668, 50669, 50670, 50671, 50672, 50673, 50674, 50675, 50676, 50677 were selected from the ORFs having the nucleotide sequence represented by SEQ ID NO: 4032.

The probes having the nucleotide sequence represented by SEQ ID NOS: 50678, 50679, 50680, 50681, 50682, 50683, 50684, 50685, 50686, 50687, 50688 were selected from the ORFs having the nucleotide sequence represented by SEQ ID NO: 4033.

The probes having the nucleotide sequence represented by SEQ ID NOS: 50689, 50690, 50691, 50692, 50693, 50694, 50695, 50696, 50697, 50698, 50699 were selected from the ORFs having the nucleotide sequence represented by SEQ ID NO: 4034.

The probes having the nucleotide sequence represented by SEQ ID NOS: 50700, 50701, 50702, 50703, 50704, 50705, 50706, 50707, 50708, 50709, 50710 were selected from the ORFs having the nucleotide sequence represented by SEQ ID NO: 4035.

The probes having the nucleotide sequence represented by SEQ ID NOS: 50711, 50712, 50713, 50714, 50715, 50716, 50717, 50718, 50719, 50720, 50721 were selected from the ORFs having the nucleotide sequence represented by SEQ ID NO: 4036.

The probes having the nucleotide sequence represented by SEQ ID NOS: 50722, 50723, 50724, 50725, 50726, 50727, 50728, 50729, 50730, 50731, 50732 were selected from the ORFs having the nucleotide sequence represented by SEQ ID NO: 4037.

The probes having the nucleotide sequence represented by SEQ ID NOS: 50733, 50734, 50735, 50736, 50737, 50738, 50739, 50740, 50741, 50742, 50743 were selected from the ORFs having the nucleotide sequence represented by SEQ ID NO: 4038.

The probes having the nucleotide sequence represented by SEQ ID NOS: 50744, 50745, 50746, 50747, 50748, 50749, 50750, 50751, 50752, 50753, 50754 were selected from the ORFs having the nucleotide sequence represented by SEQ ID NO: 4039.

The probes having the nucleotide sequence represented by SEQ ID NOS: 50755, 50756, 50757, 50758, 50759, 50760, 50761, 50762, 50763, 50764, 50765 were selected from the ORFs having the nucleotide sequence represented by SEQ ID NO: 4040.

The probes having the nucleotide sequence represented by SEQ ID NOS: 50766, 50767, 50768, 50769, 50770, 50771, 50772, 50773, 50774, 50775, 50776 were selected from the ORFs having the nucleotide sequence represented by SEQ ID NO: 4041.

The probes having the nucleotide sequence represented by SEQ ID NOS: 50777, 50778, 50779, 50780, 50781, 50782, 50783, 50784, 50785, 50786, 50787 were selected from the ORFs having the nucleotide sequence represented by SEQ ID NO: 4042.

The probes having the nucleotide sequence represented by SEQ ID NOS: 50788, 50789, 50790, 50791, 50792, 50793, 50794, 50795, 50796, 50797, 50798 were selected from the ORFs having the nucleotide sequence represented by SEQ ID NO: 4043.

The probes having the nucleotide sequence represented by SEQ ID NOS: 50799, 50800, 50801, 50802, 50803, 50804, 50805, 50806, 50807, 50808, 50809 were selected from the ORFs having the nucleotide sequence represented by SEQ ID NO: 4044.

The probes having the nucleotide sequence represented by SEQ ID NOS: 50810, 50811, 50812, 50813, 50814, 50815, 50816, 50817, 50818, 50819, 50820 were selected from the ORFs having the nucleotide sequence represented by SEQ ID NO: 4045.

The probes having the nucleotide sequence represented by SEQ ID NOS: 50821, 50822, 50823, 50824, 50825, 50826, 50827, 50828, 50829, 50830, 50831 were selected from the ORFs having the nucleotide sequence represented by SEQ ID NO: 4046.

The probes having the nucleotide sequence represented by SEQ ID NOS: 50832, 50833, 50834, 50835, 50836, 50837, 50838, 50839, 50840, 50841, 50842 were selected from the ORFs having the nucleotide sequence represented by SEQ ID NO: 4047.

The probes having the nucleotide sequence represented by SEQ ID NOS: 50843, 50844, 50845, 50846, 50847, 50848, 50849, 50850, 50851, 50852, 50853 were selected from the ORFs having the nucleotide sequence represented by SEQ ID NO: 4048.

The probes having the nucleotide sequence represented by SEQ ID NOS: 50854, 50855, 50856, 50857, 50858, 50859, 50860, 50861, 50862, 50863 were selected from the ORFs having the nucleotide sequence represented by SEQ ID NO: 4049.

The probes having the nucleotide sequence represented by SEQ ID NOS: 50864, 50865, 50866, 50867, 50868, 50869, 50870, 50871, 50872, 50873, 50874 were selected from the ORFs having the nucleotide sequence represented by SEQ ID NO: 4050.

The probes having the nucleotide sequence represented by SEQ ID NOS: 50875, 50876, 50877, 50878, 50879, 50880, 50881, 50882, 50883, 50884, 50885 were selected from the ORFs having the nucleotide sequence represented by SEQ ID NO: 4051.

The probes having the nucleotide sequence represented by SEQ ID NOS: 50886, 50887, 50888, 50889, 50890, 50891, 50892, 50893, 50894, 50895, 50896 were selected from the ORFs having the nucleotide sequence represented by SEQ ID NO: 4052.

The probes having the nucleotide sequence represented by SEQ ID NOS: 50897, 50898, 50899, 50900, 50901, 50902, 50903, 50904, 50905, 50906, 50907 were selected from the ORFs having the nucleotide sequence represented by SEQ ID NO: 4053.

The probes having the nucleotide sequence represented by SEQ ID NOS: 50908, 50909, 50910, 50911, 50912, 50913, 50914, 50915, 50916, 50917, 50918 were selected from the ORFs having the nucleotide sequence represented by SEQ ID NO: 4054.

The probes having the nucleotide sequence represented by SEQ ID NOS: 50919, 50920, 50921, 50922, 50923, 50924, 50925, 50926, 50927, 50928, 50929 were selected from the ORFs having the nucleotide sequence represented by SEQ ID NO: 4055.

The probes having the nucleotide sequence represented by SEQ ID NOS: 50930, 50931, 50932, 50933, 50934, 50935, 50936, 50937, 50938, 50939, 50940 were selected from the ORFs having the nucleotide sequence represented by SEQ ID NO: 4056.

The probes having the nucleotide sequence represented by SEQ ID NOS: 50941, 50942, 50943, 50944, 50945, 50946, 50947, 50948, 50949, 50950, 50951 were selected from the ORFs having the nucleotide sequence represented by SEQ ID NO: 4057.

The probes having the nucleotide sequence represented by SEQ ID NOS: 50952, 50953, 50954, 50955, 50956, 50957, 50958, 50959, 50960, 50961, 50962 were selected from the ORFs having the nucleotide sequence represented by SEQ ID NO: 4058.

The probes having the nucleotide sequence represented by SEQ ID NOS: 50963, 50964, 50965, 50966, 50967, 50968, 50969, 50970, 50971, 50972, 50973 were selected from the ORFs having the nucleotide sequence represented by SEQ ID NO: 4059.

The probes having the nucleotide sequence represented by SEQ ID NOS: 50974, 50975, 50976, 50977, 50978, 50979, 50980, 50981, 50982, 50983, 50984 were selected from the ORFs having the nucleotide sequence represented by SEQ ID NO: 4060.

The probes having the nucleotide sequence represented by SEQ ID NOS: 50985, 50986, 50987, 50988, 50989, 50990, 50991, 50992, 50993, 50994, 50995 were selected from the ORFs having the nucleotide sequence represented by SEQ ID NO: 4061.

The probes having the nucleotide sequence represented by SEQ ID NOS: 50996, 50997, 50998, 50999, 51000, 51001, 51002, 51003, 51004, 51005, 51006 were selected from the ORFs having the nucleotide sequence represented by SEQ ID NO: 4062.

The probes having the nucleotide sequence represented by SEQ ID NOS: 51007, 51008, 51009, 51010, 51011, 51012, 51013, 51014, 51015, 51016, 51017 were selected from the ORFs having the nucleotide sequence represented by SEQ ID NO: 4063.

The probes having the nucleotide sequence represented by SEQ ID NOS: 51018, 51019, 51020, 51021, 51022, 51023, 51024, 51025, 51026, 51027, 51028 were selected from the ORFs having the nucleotide sequence represented by SEQ ID NO: 4064.

The probes having the nucleotide sequence represented by SEQ ID NOS: 51029, 51030, 51031, 51032, 51033, 51034, 51035, 51036, 51037, 51038, 51039 were selected from the ORFs having the nucleotide sequence represented by SEQ ID NO: 4065.

The probes having the nucleotide sequence represented by SEQ ID NOS: 51040, 51041, 51042, 51043, 51044, 51045, 51046, 51047, 51048, 51049, 51050 were selected from the ORFs having the nucleotide sequence represented by SEQ ID NO: 4066.

The probes having the nucleotide sequence represented by SEQ ID NOS: 51051, 51052, 51053, 51054, 51055, 51056, 51057, 51058, 51059, 51060, 51061 were selected from the ORFs having the nucleotide sequence represented by SEQ ID NO: 4067.

The probes having the nucleotide sequence represented by SEQ ID NOS: 51062, 51063, 51064, 51065, 51066, 51067, 51068, 51069, 51070, 51071, 51072 were selected from the ORFs having the nucleotide sequence represented by SEQ ID NO: 4068.

The probes having the nucleotide sequence represented by SEQ ID NOS: 51073, 51074, 51075, 51076, 51077, 51078, 51079, 51080, 51081, 51082, 51083 were selected from the ORFs having the nucleotide sequence represented by SEQ ID NO: 4069.

The probes having the nucleotide sequence represented by SEQ ID NOS: 51084, 51085, 51086, 51087, 51088, 51089, 51090, 51091, 51092, 51093, 51094 were selected from the ORFs having the nucleotide sequence represented by SEQ ID NO: 4070.

The probes having the nucleotide sequence represented by SEQ ID NOS: 51095, 51096, 51097, 51098, 51099, 51100, 51101, 51102, 51103, 51104, 51105 were selected from the ORFs having the nucleotide sequence represented by SEQ ID NO: 4071.

The probes having the nucleotide sequence represented by SEQ ID NOS: 51106, 51107, 51108, 51109, 51110, 51111, 51112, 51113, 51114, 51115, 51116 were selected from the ORFs having the nucleotide sequence represented by SEQ ID NO: 4072.

The probes having the nucleotide sequence represented by SEQ ID NOS: 51117, 51118, 51119, 51120, 51121, 51122, 51123, 51124, 51125, 51126, 51127 were selected from the ORFs having the nucleotide sequence represented by SEQ ID NO: 4073.

The probes having the nucleotide sequence represented by SEQ ID NOS: 51128, 51129, 51130, 51131, 51132, 51133, 51134, 51135, 51136, 51137, 51138, 51139, 51140, 51141, 51142, 51143, 51144, 51145, 51146, 51147, 51148, 51149 were selected from the ORFs having the nucleotide sequence represented by SEQ ID NO: 4074.

The probes having the nucleotide sequence represented by SEQ ID NOS: 51150, 51151, 51152, 51153, 51154, 51155, 51156, 51157, 51158, 51159, 51160, 51161, 51162, 51163, 51164, 51165, 51166, 51167, 51168, 51169 were selected from the ORFs having the nucleotide sequence represented by SEQ ID NO: 4075.

The probes having the nucleotide sequence represented by SEQ ID NOS: 51170, 51171, 51172, 51173, 51174, 51175, 51176, 51177, 51178, 51179, 51180 were selected from the ORFs having the nucleotide sequence represented by SEQ ID NO: 4076.

The probes having the nucleotide sequence represented by SEQ ID NOS: 51181, 51182, 51183, 51184, 51185, 51186, 51187, 51188, 51189, 51190, 51191 were selected from the ORFs having the nucleotide sequence represented by SEQ ID NO: 4077.

The probes having the nucleotide sequence represented by SEQ ID NOS: 51192, 51193, 51194, 51195, 51196, 51197, 51198, 51199, 51200, 51201, 51202 were selected from the ORFs having the nucleotide sequence represented by SEQ ID NO: 4078.

The probes having the nucleotide sequence represented by SEQ ID NOS: 51203, 51204, 51205, 51206, 51207, 51208, 51209, 51210, 51211, 51212, 51213 were selected from the ORFs having the nucleotide sequence represented by SEQ ID NO: 4079.

The probes having the nucleotide sequence represented by SEQ ID NOS: 51214, 51215, 51216, 51217, 51218, 51219, 51220, 51221, 51222, 51223, 51224 were selected from the ORFs having the nucleotide sequence represented by SEQ ID NO: 4080.

The probes having the nucleotide sequence represented by SEQ ID NOS: 51225, 51226, 51227, 51228, 51229, 51230, 51231, 51232, 51233, 51234, 51235 were selected from the ORFs having the nucleotide sequence represented by SEQ ID NO: 4081.

The probes having the nucleotide sequence represented by SEQ ID NOS: 51236, 51237, 51238, 51239, 51240, 51241, 51242, 51243, 51244, 51245, 51246 were selected from the ORFs having the nucleotide sequence represented by SEQ ID NO: 4082.

The probes having the nucleotide sequence represented by SEQ ID NOS: 51247, 51248, 51249, 51250, 51251, 51252, 51253, 51254, 51255, 51256, 51257 were selected from the ORFs having the nucleotide sequence represented by SEQ ID NO: 4083.

The probes having the nucleotide sequence represented by SEQ ID NOS: 51258, 51259, 51260, 51261, 51262, 51263, 51264, 51265, 51266, 51267, 51268 were selected from the ORFs having the nucleotide sequence represented by SEQ ID NO: 4084.

The probes having the nucleotide sequence represented by SEQ ID NOS: 51269, 51270, 51271, 51272, 51273, 51274, 51275, 51276, 51277, 51278, 51279 were selected from the ORFs having the nucleotide sequence represented by SEQ ID NO: 4085.

The probes having the nucleotide sequence represented by SEQ ID NOS: 51280, 51281, 51282, 51283, 51284, 51285, 51286, 51287, 51288, 51289, 51290 were selected from the ORFs having the nucleotide sequence represented by SEQ ID NO: 4086.

The probes having the nucleotide sequence represented by SEQ ID NOS: 51291, 51292, 51293, 51294, 51295, 51296, 51297, 51298, 51299, 51300, 51301 were selected from the ORFs having the nucleotide sequence represented by SEQ ID NO: 4087.

The probes having the nucleotide sequence represented by SEQ ID NOS: 51302, 51303, 51304, 51305, 51306, 51307, 51308, 51309, 51310, 51311, 51312 were selected from the ORFs having the nucleotide sequence represented by SEQ ID NO: 4088.

The probes having the nucleotide sequence represented by SEQ ID NOS: 51313, 51314, 51315, 51316, 51317, 51318, 51319, 51320, 51321, 51322, 51323 were selected from the ORFs having the nucleotide sequence represented by SEQ ID NO: 4089.

The probes having the nucleotide sequence represented by SEQ ID NOS: 51324, 51325, 51326, 51327, 51328, 51329, 51330, 51331, 51332, 51333, 51334 were selected from the ORFs having the nucleotide sequence represented by SEQ ID NO: 4090.

The probes having the nucleotide sequence represented by SEQ ID NOS: 51335, 51336, 51337, 51338, 51339, 51340, 51341, 51342, 51343, 51344, 51345 were selected from the ORFs having the nucleotide sequence represented by SEQ ID NO: 4091.

The probes having the nucleotide sequence represented by SEQ ID NOS: 51346, 51347, 51348, 51349, 51350, 51351, 51352, 51353, 51354, 51355, 51356 were selected from the ORFs having the nucleotide sequence represented by SEQ ID NO: 4092.

The probes having the nucleotide sequence represented by SEQ ID NOS: 51357, 51358, 51359, 51360, 51361, 51362, 51363, 51364, 51365, 51366, 51367 were selected from the ORFs having the nucleotide sequence represented by SEQ ID NO: 4093.

The probes having the nucleotide sequence represented by SEQ ID NOS: 51368, 51369, 51370, 51371, 51372, 51373, 51374, 51375, 51376, 51377, 51378 were selected from the ORFs having the nucleotide sequence represented by SEQ ID NO: 4094.

The probes having the nucleotide sequence represented by SEQ ID NOS: 51379, 51380, 51381, 51382, 51383, 51384, 51385, 51386, 51387, 51388, 51389 were selected from the ORFs having the nucleotide sequence represented by SEQ ID NO: 4095.

The probes having the nucleotide sequence represented by SEQ ID NOS: 51390, 51391, 51392, 51393, 51394, 51395, 51396, 51397, 51398, 51399, 51400 were selected from the ORFs having the nucleotide sequence represented by SEQ ID NO: 4096.

The probes having the nucleotide sequence represented by SEQ ID NOS: 51401, 51402, 51403, 51404, 51405, 51406, 51407, 51408, 51409, 51410, 51411 were selected from the ORFs having the nucleotide sequence represented by SEQ ID NO: 4097.

The probes having the nucleotide sequence represented by SEQ ID NOS: 51412, 51413, 51414, 51415, 51416, 51417, 51418, 51419, 51420, 51421, 51422 were selected from the ORFs having the nucleotide sequence represented by SEQ ID NO: 4098.

The probes having the nucleotide sequence represented by SEQ ID NOS: 51423, 51424, 51425, 51426, 51427, 51428, 51429, 51430, 51431, 51432, 51433 were selected from the ORFs having the nucleotide sequence represented by SEQ ID NO: 4099.

The probes having the nucleotide sequence represented by SEQ ID NOS: 51434, 51435, 51436, 51437, 51438, 51439, 51440, 51441, 51442, 51443, 51444 were selected from the ORFs having the nucleotide sequence represented by SEQ ID NO: 4100.

The probes having the nucleotide sequence represented by SEQ ID NOS: 51445, 51446, 51447, 51448, 51449, 51450, 51451, 51452, 51453, 51454, 51455 were selected from the ORFs having the nucleotide sequence represented by SEQ ID NO: 4101.

The probes having the nucleotide sequence represented by SEQ ID NOS: 5.1456, 51457, 51458, 51459, 51460, 51461, 51462, 51463, 51464, 51465, 51466 were selected from the ORFs having the nucleotide sequence represented by SEQ ID NO: 4102.

The probes having the nucleotide sequence represented by SEQ ID NOS: 51467, 51468, 51469, 51470, 51471, 51472, 51473, 51474, 51475, 51476, 51477 were selected from the ORFs having the nucleotide sequence represented by SEQ ID NO: 4103.

The probes having the nucleotide sequence represented by SEQ ID NOS: 51478, 51479, 51480, 51481, 51482, 51483, 51484, 51485, 51486, 51487, 51488 were selected from the ORFs having the nucleotide sequence represented by SEQ ID NO: 4104.

The probes having the nucleotide sequence represented by SEQ ID NOS: 51489, 51490, 51491, 51492, 51493, 51494, 51495, 51496, 51497, 51498, 51499 were selected from the ORFs having the nucleotide sequence represented by SEQ ID NO: 4105.

The probes having the nucleotide sequence represented by SEQ ID NOS: 51500, 51501, 51502, 51503, 51504, 51505, 51506, 51507, 51508, 51509, 51510 were selected from the ORFs having the nucleotide sequence represented by SEQ ID NO: 4106.

The probes having the nucleotide sequence represented by SEQ ID NOS: 51511, 51512, 51513, 51514, 51515, 51516, 51517, 51518, 51519, 51520, 51521 were selected from the ORFs having the nucleotide sequence represented by SEQ ID NO: 4107.

The probes having the nucleotide sequence represented by SEQ ID NOS: 51522, 51523, 51524, 51525, 51526, 51527, 51528, 51529, 51530, 51531, 51532 were selected from the ORFs having the nucleotide sequence represented by SEQ ID NO: 4108.

The probes having the nucleotide sequence represented by SEQ ID NOS: 51533, 51534, 51535, 51536, 51537, 51538, 51539, 51540, 51541, 51542, 51543 were selected from the ORFs having the nucleotide sequence represented by SEQ ID NO: 4109.

The probes having the nucleotide sequence represented by SEQ ID NOS: 51544, 51545, 51546, 51547, 51548, 51549, 51550, 51551, 51552, 51553, 51554 were selected from the ORFs having the nucleotide sequence represented by SEQ ID NO: 4110.

The probes having the nucleotide sequence represented by SEQ ID NOS: 51555, 51556, 51557, 51558, 51559, 51560, 51561, 51562, 51563, 51564, 51565 were selected from the ORFs having the nucleotide sequence represented by SEQ ID NO: 4111.

The probes having the nucleotide sequence represented by SEQ ID NOS: 51566, 51567, 51568, 51569, 51570, 51571, 51572, 51573, 51574, 51575, 51576 were selected from the ORFs having the nucleotide sequence represented by SEQ ID NO: 4112.

The probes having the nucleotide sequence represented by SEQ ID NOS: 51577, 51578, 51579, 51580, 51581, 51582, 51583, 51584, 51585, 51586, 51587 were selected from the ORFs having the nucleotide sequence represented by SEQ ID NO: 4113.

The probes having the nucleotide sequence represented by SEQ ID NOS: 51588, 51589, 51590, 51591, 51592, 51593, 51594, 51595, 51596, 51597, 51598 were selected from the ORFs having the nucleotide sequence represented by SEQ ID NO: 4114.

The probes having the nucleotide sequence represented by SEQ ID NOS: 51599, 51600, 51601, 51602, 51603, 51604, 51605, 51606, 51607, 51608, 51609 were selected from the ORFs having the nucleotide sequence represented by SEQ ID NO: 4115.

The probes having the nucleotide sequence represented by SEQ ID NOS: 51610, 51611, 51612, 51613, 51614, 51615, 51616, 51617, 51618, 51619, 51620 were selected from the ORFs having the nucleotide sequence represented by SEQ ID NO: 4116.

The probes having the nucleotide sequence represented by SEQ ID NOS: 51621, 51622, 51623, 51624, 51625, 51626, 51627, 51628, 51629, 51630, 51631 were selected from the ORFs having the nucleotide sequence represented by SEQ ID NO: 4117.

The probes having the nucleotide sequence represented by SEQ ID NOS: 51632, 51633, 51634, 51635, 51636, 51637, 51638, 51639, 51640, 51641, 51642 were selected from the ORFs having the nucleotide sequence represented by SEQ ID NO: 4118.

The probes having the nucleotide sequence represented by SEQ ID NOS: 51643, 51644, 51645, 51646, 51647, 51648, 51649, 51650, 51651, 51652, 51653 were selected from the ORFs having the nucleotide sequence represented by SEQ ID NO: 4119.

The probes having the nucleotide sequence represented by SEQ ID NOS: 51654, 51655, 51656, 51657, 51658, 51659, 51660, 51661, 51662, 51663, 51664 were selected from the ORFs having the nucleotide sequence represented by SEQ ID NO: 4120.

The probes having the nucleotide sequence represented by SEQ ID NOS: 51665, 51666, 51667, 51668, 51669, 51670, 51671, 51672, 51673, 51674, 51675 were selected from the ORFs having the nucleotide sequence represented by SEQ ID NO: 4121.

The probes having the nucleotide sequence represented by SEQ ID NOS: 51676, 51677, 51678, 51679, 51680, 51681, 51682, 51683, 51684, 51685, 51686 were selected from the ORFs having the nucleotide sequence represented by SEQ ID NO: 4122.

The probes having the nucleotide sequence represented by SEQ ID NOS: 51687, 51688, 51689, 51690, 51691, 51692, 51693, 51694, 51695, 51696, 51697 were selected from the ORFs having the nucleotide sequence represented by SEQ ID NO: 4123.

The probes having the nucleotide sequence represented by SEQ ID NOS: 51698, 51699, 51700, 51701, 51702, 51703, 51704, 51705, 51706, 51707, 51708 were selected from the ORFs having the nucleotide sequence represented by SEQ ID NO: 4124.

The probes having the nucleotide sequence represented by SEQ ID NOS: 51709, 51710, 51711, 51712, 51713, 51714, 51715, 51716, 51717, 51718, 51719 were selected from the ORFs having the nucleotide sequence represented by SEQ ID NO: 4125.

The probes having the nucleotide sequence represented by SEQ ID NOS: 51720, 51721, 51722, 51723, 51724, 51725, 51726, 51727, 51728, 51729, 51730 were selected from the ORFs having the nucleotide sequence represented by SEQ ID NO: 4126.

The probes having the nucleotide sequence represented by SEQ ID NOS: 51731, 51732, 51733, 51734, 51735, 51736, 51737, 51738, 51739, 51740, 51741 were selected from the ORFs having the nucleotide sequence represented by SEQ ID NO: 4127.

The probes having the nucleotide sequence represented by SEQ ID NOS: 51742, 51743, 51744, 51745, 51746, 51747, 51748, 51749, 51750, 51751, 51752 were selected from the ORFs having the nucleotide sequence represented by SEQ ID NO: 4128.

The probes having the nucleotide sequence represented by SEQ ID NOS: 51753, 51754, 51755, 51756, 51757, 51758, 51759, 51760, 51761, 51762, 51763 were selected from the ORFs having the nucleotide sequence represented by SEQ ID NO: 4129.

The probes having the nucleotide sequence represented by SEQ ID NOS: 51764, 51765, 51766, 51767, 51768, 51769, 51770, 51771, 51772, 51773, 51774 were selected from the ORFs having the nucleotide sequence represented by SEQ ID NO: 4130.

The probes having the nucleotide sequence represented by SEQ ID NOS: 51775, 51776, 51777, 51778, 51779, 51780, 51781, 51782, 51783, 51784, 51785 were selected from the ORFs having the nucleotide sequence represented by SEQ ID NO: 4131.

The probes having the nucleotide sequence represented by SEQ ID NOS: 51786, 51787, 51788, 51789, 51790, 51791, 51792, 51793, 51794, 51795, 51796 were selected from the ORFs having the nucleotide sequence represented by SEQ ID NO: 4132.

The probes having the nucleotide sequence represented by SEQ ID NOS: 51797, 51798, 51799, 51800, 51801, 51802, 51803, 51804, 51805, 51806, 51807 were selected from the ORFs having the nucleotide sequence represented by SEQ ID NO: 4133.

The probes having the nucleotide sequence represented by SEQ ID NOS: 51808, 51809, 51810, 51811, 51812, 51813, 51814, 51815, 51816, 51817, 51818 were selected from the ORFs having the nucleotide sequence represented by SEQ ID NO: 4134.

The probes having the nucleotide sequence represented by SEQ ID NOS: 51819, 51820, 51821, 51822, 51823, 51824, 51825, 51826, 51827, 51828, 51829 were selected from the ORFs having the nucleotide sequence represented by SEQ ID NO: 4135.

The probes having the nucleotide sequence represented by SEQ ID NOS: 51830, 51831, 51832, 51833, 51834, 51835, 51836, 51837, 51838, 51839, 51840 were selected from the ORFs having the nucleotide sequence represented by SEQ ID NO: 4136.

The probes having the nucleotide sequence represented by SEQ ID NOS: 51841, 51842, 51843, 51844, 51845, 51846, 51847, 51848, 51849, 51850, 51851, 51852, 51853, 51854, 51855, 51856, 51857, 51858, 51859, 51860, 51861, 51862 were selected from the ORFs having the nucleotide sequence represented by SEQ ID NO: 4137.

The probes having the nucleotide sequence represented by SEQ ID NOS: 51863, 51864, 51865, 51866, 51867, 51868, 51869, 51870, 51871, 51872, 51873 were selected from the ORFs having the nucleotide sequence represented by SEQ ID NO: 4138.

The probes having the nucleotide sequence represented by SEQ ID NOS: 51874, 51875, 51876, 51877, 51878, 51879, 51880, 51881, 51882, 51883, 51884 were selected from the ORFs having the nucleotide sequence represented by SEQ ID NO: 4139.

The probes having the nucleotide sequence represented by SEQ ID NOS: 51885, 51886, 51887, 51888, 51889, 51890, 51891, 51892, 51893, 51894, 51895 were selected from the ORFs having the nucleotide sequence represented by SEQ ID NO: 4140.

The probes having the nucleotide sequence represented by SEQ ID NOS: 51896, 51897, 51898, 51899, 51900, 51901, 51902, 51903, 51904, 51905, 51906 were selected from the ORFs having the nucleotide sequence represented by SEQ ID NO: 4141.

The probes having the nucleotide sequence represented by SEQ ID NOS: 51907, 51908, 51909, 51910, 51911, 51912, 51913, 51914, 51915, 51916, 51917 were selected from the ORFs having the nucleotide sequence represented by SEQ ID NO: 4142.

The probes having the nucleotide sequence represented by SEQ ID NOS: 51918, 51919, 51920, 51921, 51922, 51923, 51924, 51925, 51926, 51927, 51928 were selected from the ORFs having the nucleotide sequence represented by SEQ ID NO: 4143.

The probes having the nucleotide sequence represented by SEQ ID NOS: 51929, 51930, 51931, 51932, 51933, 51934, 51935, 51936, 51937, 51938, 51939 were selected from the ORFs having the nucleotide sequence represented by SEQ ID NO: 4144.

The probes having the nucleotide sequence represented by SEQ ID NOS: 51940, 51941, 51942, 51943, 51944, 51945, 51946, 51947, 51948, 51949, 51950 were selected from the ORFs having the nucleotide sequence represented by SEQ ID NO: 4145.

The probes having the nucleotide sequence represented by SEQ ID NOS: 51951, 51952, 51953, 51954, 51955, 51956, 51957, 51958, 51959, 51960, 51961 were selected from the ORFs having the nucleotide sequence represented by SEQ ID NO: 4146.

The probes having the nucleotide sequence represented by SEQ ID NOS: 51962, 51963, 51964, 51965, 51966, 51967, 51968, 51969, 51970, 51971, 51972 were selected from the ORFs having the nucleotide sequence represented by SEQ ID NO: 4147.

The probes having the nucleotide sequence represented by SEQ ID NOS: 51973, 51974, 51975, 51976, 51977, 51978, 51979, 51980, 51981, 51982, 51983 were selected from the ORFs having the nucleotide sequence represented by SEQ ID NO: 4148.

The probes having the nucleotide sequence represented by SEQ ID NOS: 51984, 51985, 51986, 51987, 51988, 51989, 51990, 51991, 51992, 51993, 51994 were selected from the ORFs having the nucleotide sequence represented by SEQ ID NO: 4149.

The probes having the nucleotide sequence represented by SEQ ID NOS: 51995, 51996, 51997, 51998, 51999, 52000, 52001, 52002, 52003, 52004, 52005 were selected from the ORFs having the nucleotide sequence represented by SEQ ID NO: 4150.

The probes having the nucleotide sequence represented by SEQ ID NOS: 52006, 52007, 52008, 52009, 52010, 52011, 52012, 52013, 52014, 52015, 52016 were selected from the ORFs having the nucleotide sequence represented by SEQ ID NO: 4151.

The probes having the nucleotide sequence represented by SEQ ID NOS: 52017, 52018, 52019, 52020, 52021, 52022, 52023, 52024, 52025, 52026, 52027 were selected from the ORFs having the nucleotide sequence represented by SEQ ID NO: 4152.

The probes having the nucleotide sequence represented by SEQ ID NOS: 52028, 52029, 52030, 52031, 52032, 52033, 52034, 52035, 52036, 52037, 52038 were selected from the ORFs having the nucleotide sequence represented by SEQ ID NO: 4153.

The probes having the nucleotide sequence represented by SEQ ID NOS: 52039, 52040, 52041, 52042, 52043, 52044, 52045, 52046, 52047, 52048, 52049 were selected from the ORFs having the nucleotide sequence represented by SEQ ID NO: 4154.

The probes having the nucleotide sequence represented by SEQ ID NOS: 52050, 52051, 52052, 52053, 52054, 52055, 52056, 52057, 52058, 52059, 52060 were selected from the ORFs having the nucleotide sequence represented by SEQ ID NO: 4155.

The probes having the nucleotide sequence represented by SEQ ID NOS: 52061, 52062, 52063, 52064, 52065, 52066, 52067, 52068, 52069, 52070, 52071 were selected from the ORFs having the nucleotide sequence represented by SEQ ID NO: 4156.

The probes having the nucleotide sequence represented by SEQ ID NOS: 52072, 52073, 52074, 52075, 52076, 52077, 52078, 52079, 52080, 52081, 52082 were selected from the ORFs having the nucleotide sequence represented by SEQ ID NO: 4157.

The probes having the nucleotide sequence represented by SEQ ID NOS: 52083, 52084, 52085, 52086, 52087, 52088, 52089, 52090, 52091, 52092, 52093 were selected from the ORFs having the nucleotide sequence represented by SEQ ID NO: 4158.

The probes having the nucleotide sequence represented by SEQ ID NOS: 52094, 52095, 52096, 52097, 52098, 52099, 52100, 52101, 52102, 52103, 52104 were selected from the ORFs having the nucleotide sequence represented by SEQ ID NO: 4159.

The probes having the nucleotide sequence represented by SEQ ID NOS: 52105, 52106, 52107, 52108, 52109, 52110, 52111, 52112, 52113, 52114, 52115 were selected from the ORFs having the nucleotide sequence represented by SEQ ID NO: 4160.

The probes having the nucleotide sequence represented by SEQ ID NOS: 52116, 52117, 52118, 52119, 52120, 52121, 52122, 52123, 52124, 52125, 52126 were selected from the ORFs having the nucleotide sequence represented by SEQ ID NO: 4161.

The probes having the nucleotide sequence represented by SEQ ID NOS: 52127, 52128, 52129, 52130, 52131, 52132, 52133, 52134, 52135, 52136, 52137 were selected from the ORFs having the nucleotide sequence represented by SEQ ID NO: 4162.

The probes having the nucleotide sequence represented by SEQ ID NOS: 52138, 52139, 52140, 52141, 52142, 52143, 52144, 52145, 52146, 52147, 52148 were selected from the ORFs having the nucleotide sequence represented by SEQ ID NO: 4163.

The probes having the nucleotide sequence represented by SEQ ID NOS: 52149, 52150, 52151, 52152, 52153, 52154, 52155, 52156, 52157, 52158, 52159 were selected from the ORFs having the nucleotide sequence represented by SEQ ID NO: 4164.

The probes having the nucleotide sequence represented by SEQ ID NOS: 52160, 52161, 52162, 52163, 52164, 52165, 52166, 52167, 52168, 52169, 52170 were selected from the ORFs having the nucleotide sequence represented by SEQ ID NO: 4165.

The probes having the nucleotide sequence represented by SEQ ID NOS: 52171, 52172, 52173, 52174, 52175, 52176, 52177, 52178, 52179, 52180, 52181 were selected from the ORFs having the nucleotide sequence represented by SEQ ID NO: 4166.

The probes having the nucleotide sequence represented by SEQ ID NOS: 52182, 52183, 52184, 52185, 52186, 52187, 52188, 52189, 52190, 52191, 52192 were selected from the ORFs having the nucleotide sequence represented by SEQ ID NO: 4167.

The probes having the nucleotide sequence represented by SEQ ID NOS: 52193, 52194, 52195, 52196, 52197, 52198, 52199, 52200, 52201, 52202, 52203 were selected from the ORFs having the nucleotide sequence represented by SEQ ID NO: 4168.

The probes having the nucleotide sequence represented by SEQ ID NOS: 52204, 52205, 52206, 52207, 52208, 52209, 52210, 52211, 52212, 52213, 52214 were selected from the ORFs having the nucleotide sequence represented by SEQ ID NO: 4169.

The probes having the nucleotide sequence represented by SEQ ID NOS: 52215, 52216, 52217, 52218, 52219, 52220, 52221, 52222, 52223, 52224, 52225 were selected from the ORFs having the nucleotide sequence represented by SEQ ID NO: 4170.

The probes having the nucleotide sequence represented by SEQ ID NOS: 52226, 52227, 52228, 52229, 52230, 52231, 52232, 52233, 52234, 52235, 52236 were selected from the ORFs having the nucleotide sequence represented by SEQ ID NO: 4171.

The probes having the nucleotide sequence represented by SEQ ID NOS: 52237, 52238, 52239, 52240, 52241, 52242, 52243, 52244, 52245, 52246, 52247 were selected from the ORFs having the nucleotide sequence represented by SEQ ID NO: 4172.

The probes having the nucleotide sequence represented by SEQ ID NOS: 52248, 52249, 52250, 52251, 52252, 52253, 52254, 52255, 52256, 52257, 52258 were selected from the ORFs having the nucleotide sequence represented by SEQ ID NO: 4173.

The probes having the nucleotide sequence represented by SEQ ID NOS: 52259, 52260, 52261, 52262, 52263, 52264, 52265, 52266, 52267, 52268, 52269 were selected from the ORFs having the nucleotide sequence represented by SEQ ID NO: 4174.

The probes having the nucleotide sequence represented by SEQ ID NOS: 52270, 52271, 52272, 52273, 52274, 52275, 52276, 52277, 52278, 52279, 52280 were selected from the ORFs having the nucleotide sequence represented by SEQ ID NO: 4175.

The probes having the nucleotide sequence represented by SEQ ID NOS: 52281, 52282, 52283, 52284, 52285, 52286, 52287, 52288, 52289, 52290, 52291 were selected from the ORFs having the nucleotide sequence represented by SEQ ID NO: 4176.

The probes having the nucleotide sequence represented by SEQ ID NOS: 52292, 52293, 52294, 52295, 52296, 52297, 52298, 52299, 52300, 52301, 52302, 52303, 52304, 52305, 52306, 52307, 52308, 52309, 52310, 52311, 52312, 52313 were selected from the ORFs having the nucleotide sequence represented by SEQ ID NO: 4177.

The probes having the nucleotide sequence represented by SEQ ID NOS: 52314, 52315, 52316, 52317, 52318, 52319, 52320, 52321, 52322, 52323, 52324 were selected from the ORFs having the nucleotide sequence represented by SEQ ID NO: 4178.

The probes having the nucleotide sequence represented by SEQ ID NOS: 52325, 52326, 52327, 52328, 52329, 52330, 52331, 52332, 52333, 52334, 52335 were selected from the ORFs having the nucleotide sequence represented by SEQ ID NO: 4179.

The probes having the nucleotide sequence represented by SEQ ID NOS: 52336, 52337, 52338, 52339, 52340, 52341, 52342, 52343, 52344, 52345, 52346 were selected from the ORFs having the nucleotide sequence represented by SEQ ID NO: 4180.

The probes having the nucleotide sequence represented by SEQ ID NOS: 52347, 52348, 52349, 52350, 52351, 52352, 52353, 52354, 52355, 52356, 52357 were selected from the ORFs having the nucleotide sequence represented by SEQ ID NO: 4181.

The probes having the nucleotide sequence represented by SEQ ID NOS: 52358, 52359, 52360, 52361, 52362, 52363, 52364, 52365, 52366, 52367, 52368 were selected from the ORFs having the nucleotide sequence represented by SEQ ID NO: 4182.

The probes having the nucleotide sequence represented by SEQ ID NOS: 52369, 52370, 52371, 52372, 52373, 52374, 52375, 52376, 52377, 52378, 52379 were selected from the ORFs having the nucleotide sequence represented by SEQ ID NO: 4183.

The probes having the nucleotide sequence represented by SEQ ID NOS: 52380, 52381, 52382, 52383, 52384, 52385, 52386, 52387, 52388, 52389, 52390 were selected from the ORFs having the nucleotide sequence represented by SEQ ID NO: 4184.

The probes having the nucleotide sequence represented by SEQ ID NOS: 52391, 52392, 52393, 52394, 52395, 52396, 52397, 52398, 52399, 52400, 52401 were selected from the ORFs having the nucleotide sequence represented by SEQ ID NO: 4185.

The probes having the nucleotide sequence represented by SEQ ID NOS: 52402, 52403, 52404, 52405, 52406, 52407, 52408, 52409, 52410, 52411, 52412 were selected from the ORFs having the nucleotide sequence represented by SEQ ID NO: 4186.

The probes having the nucleotide sequence represented by SEQ ID NOS: 52413, 52414, 52415, 52416, 52417, 52418, 52419, 52420, 52421, 52422, 52423 were selected from the ORFs having the nucleotide sequence represented by SEQ ID NO: 4187.

The probes having the nucleotide sequence represented by SEQ ID NOS: 52424, 52425, 52426, 52427, 52428, 52429, 52430, 52431, 52432, 52433, 52434 were selected from the ORFs having the nucleotide sequence represented by SEQ ID NO: 4188.

The probes having the nucleotide sequence represented by SEQ ID NOS: 52435, 52436, 52437, 52438, 52439, 52440, 52441, 52442, 52443, 52444, 52445 were selected from the ORFs having the nucleotide sequence represented by SEQ ID NO: 4189.

The probes having the nucleotide sequence represented by SEQ ID NOS: 52446, 52447, 52448, 52449, 52450, 52451, 52452, 52453, 52454, 52455, 52456 were selected from the ORFs having the nucleotide sequence represented by SEQ ID NO: 4190.

The probes having the nucleotide sequence represented by SEQ ID NOS: 52457, 52458, 52459, 52460, 52461, 52462, 52463, 52464, 52465, 52466, 52467 were selected from the ORFs having the nucleotide sequence represented by SEQ ID NO: 4191.

The probes having the nucleotide sequence represented by SEQ ID NOS: 52468, 52469, 52470, 52471, 52472, 52473, 52474, 52475, 52476, 52477, 52478 were selected from the ORFs having the nucleotide sequence represented by SEQ ID NO: 4192.

The probes having the nucleotide sequence represented by SEQ ID NOS: 52479, 52480, 52481, 52482, 52483, 52484, 52485, 52486, 52487, 52488, 52489 were selected from the ORFs having the nucleotide sequence represented by SEQ ID NO: 4193.

The probes having the nucleotide sequence represented by SEQ ID NOS: 52490, 52491, 52492, 52493, 52494, 52495, 52496, 52497, 52498, 52499, 52500 were selected from the ORFs having the nucleotide sequence represented by SEQ ID NO: 4194.

The probes having the nucleotide sequence represented by SEQ ID NOS: 52501, 52502, 52503, 52504, 52505, 52506, 52507, 52508, 52509, 52510, 52511 were selected from the ORFs having the nucleotide sequence represented by SEQ ID NO: 4195.

The probes having the nucleotide sequence represented by SEQ ID NOS: 52512, 52513, 52514, 52515, 52516, 52517, 52518, 52519, 52520, 52521, 52522 were selected from the ORFs having the nucleotide sequence represented by SEQ ID NO: 4196.

The probes having the nucleotide sequence represented by SEQ ID NOS: 52523, 52524, 52525, 52526, 52527, 52528, 52529, 52530, 52531, 52532, 52533 were selected from the ORFs having the nucleotide sequence represented by SEQ ID NO: 4197.

The probes having the nucleotide sequence represented by SEQ ID NOS: 52534, 52535, 52536, 52537, 52538, 52539, 52540, 52541, 52542, 52543, 52544 were selected from the ORFs having the nucleotide sequence represented by. SEQ ID NO: 4198.

The probes having the nucleotide sequence represented by SEQ ID NOS: 52545, 52546, 52547, 52548, 52549, 52550, 52551, 52552, 52553, 52554, 52555 were selected from the ORFs having the nucleotide sequence represented by SEQ ID NO: 4199.

The probes having the nucleotide sequence represented by SEQ ID NOS: 52556, 52557, 52558, 52559, 52560, 52561, 52562, 52563, 52564, 52565, 52566 were selected from the ORFs having the nucleotide sequence represented by SEQ ID NO: 4200.

The probes having the nucleotide sequence represented by SEQ ID NOS: 52567, 52568, 52569, 52570, 52571, 52572, 52573, 52574, 52575, 52576, 52577 were selected from the ORFs having the nucleotide sequence represented by SEQ ID NO: 4201.

The probes having the nucleotide sequence represented by SEQ ID NOS: 52578, 52579, 52580, 52581, 52582, 52583, 52584, 52585, 52586, 52587, 52588 were selected from the ORFs having the nucleotide sequence represented by SEQ ID NO: 4202.

The probes having the nucleotide sequence represented by SEQ ID NOS: 52589, 52590, 52591, 52592, 52593, 52594, 52595, 52596, 52597, 52598, 52599 were selected from the ORFs having the nucleotide sequence represented by SEQ ID NO: 4203.

The probes having the nucleotide sequence represented by SEQ ID NOS: 52600, 52601, 52602, 52603, 52604, 52605, 52606, 52607, 52608, 52609, 52610 were selected from the ORFs having the nucleotide sequence represented by SEQ ID NO: 4204.

The probes having the nucleotide sequence represented by SEQ ID NOS: 52611, 52612, 52613, 52614, 52615, 52616, 52617, 52618, 52619, 52620, 52621 were selected from the ORFs having the nucleotide sequence represented by SEQ ID NO: 4205.

The probes having the nucleotide sequence represented by SEQ ID NOS: 52622, 52623, 52624, 52625, 52626, 52627, 52628, 52629, 52630, 52631, 52632 were selected from the ORFs having the nucleotide sequence represented by SEQ ID NO: 4206.

The probes having the nucleotide sequence represented by SEQ ID NOS: 52633, 52634, 52635, 52636, 52637, 52638, 52639, 52640, 52641, 52642, 52643 were selected from the ORFs having the nucleotide sequence represented by SEQ ID NO: 4207.

The probes having the nucleotide sequence represented by SEQ ID NOS: 52644, 52645, 52646, 52647, 52648, 52649, 52650, 52651, 52652, 52653, 52654 were selected from the ORFs having the nucleotide sequence represented by SEQ ID NO: 4208.

The probes having the nucleotide sequence represented by SEQ ID NOS: 52655, 52656, 52657,: 52658, 52659, 52660, 52661, 52662, 52663, 52664, 52665 were selected from the ORFs having the nucleotide sequence represented by SEQ ID NO: 4209.

The probes having the nucleotide sequence represented by SEQ ID NOS: 52666, 52667, 52668, 52669, 52670, 52671, 52672, 52673, 52674, 52675, 52676 were selected from the ORFs having the nucleotide sequence represented by SEQ ID NO: 4210.

The probes having the nucleotide sequence represented by SEQ ID NOS: 52677, 52678, 52679, 52680, 52681, 52682, 52683, 52684, 52685, 52686, 52687 were selected from the ORFs having the nucleotide sequence represented by SEQ ID NO: 4211.

The probes having the nucleotide sequence represented by SEQ ID NOS: 52688, 52689, 52690, 52691, 52692, 52693, 52694, 52695, 52696, 52697, 52698 were selected from the ORFs having the nucleotide sequence represented by SEQ ID NO: 4212.

The probes having the nucleotide sequence represented by SEQ ID NOS: 52699, 52700, 52701, 52702, 52703, 52704, 52705, 52706, 52707, 52708, 52709 were selected from the ORFs having the nucleotide sequence represented by SEQ ID NO: 4213.

The probes having the nucleotide sequence represented by SEQ ID NOS: 52710, 52711, 52712, 52713, 52714, 52715, 52716, 52717, 52718, 52719, 52720 were selected from the ORFs having the nucleotide sequence represented by SEQ ID NO: 4214.

The probes having the nucleotide sequence represented by SEQ ID NOS: 52721, 52722, 52723, 52724, 52725, 52726, 52727, 52728, 52729, 52730, 52731 were selected from the ORFs having the nucleotide sequence represented by SEQ ID NO: 4215.

The probes having the nucleotide sequence represented by SEQ ID NOS: 52732, 52733, 52734, 52735, 52736, 52737, 52738, 5,2739, 52740, 52741, 52742 were selected from the ORFs having the nucleotide sequence represented by SEQ ID NO: 4216.

The probes having the nucleotide sequence represented by SEQ ID NOS: 52743, 52744, 52745, 52746, 52747, 52748, 52749, 52750, 52751, 52752, 52753 were selected from the ORFs having the nucleotide sequence represented by SEQ ID NO: 4217.

The probes having the nucleotide sequence represented by SEQ ID NOS: 52754, 52755, 52756, 52757, 52758, 52759, 52760, 52761, 52762, 52763, 52764 were selected from the ORFs having the nucleotide sequence represented by SEQ ID NO: 4218.

The probes having the nucleotide sequence represented by SEQ ID NOS: 52765, 52766, 52767, 52768, 52769, 52770, 52771, 52772, 52773, 52774, 52775 were selected from the ORFs having the nucleotide sequence represented by SEQ ID NO: 4219.

The probes having the nucleotide sequence represented by SEQ ID NOS: 52776, 52777, 52778, 52779, 52780, 52781, 52782, 52783, 52784, 52785, 52786 were selected from the ORFs having the nucleotide sequence represented by SEQ ID NO: 4220.

The probes having the nucleotide sequence represented by SEQ ID NOS: 52787, 52788, 52789, 52790, 52791, 52792, 52793, 52794, 52795, 52796, 52797 were selected from the ORFs having the nucleotide sequence represented by SEQ ID NO: 4221.

The probes having the nucleotide sequence represented by SEQ ID NOS: 52798, 52799, 52800, 52801, 52802, 52803, 52804, 52805, 52806, 52807, 52808 were selected from the ORFs having the nucleotide sequence represented by SEQ ID NO: 4222.

The probes having the nucleotide sequence represented by SEQ ID NOS: 52809, 52810, 52811, 52812, 52813, 52814, 52815, 52816, 52817, 52818, 52819 were selected from the ORFs having the nucleotide sequence represented by SEQ ID NO: 4223.

The probes having the nucleotide sequence represented by SEQ ID NOS: 52820, 52821, 52822, 52823, 52824, 52825, 52826, 52827, 52828, 52829, 52830 were selected from the ORFs having the nucleotide sequence represented by SEQ ID NO: 4224.

The probes having the nucleotide sequence represented by SEQ ID NOS: 52831, 52832, 52833, 52834, 52835, 52836, 52837, 52838, 52839, 52840, 52841 were selected from the ORFs having the nucleotide sequence represented by SEQ ID NO: 4225.

The probes having the nucleotide sequence represented by SEQ ID NOS: 52842, 52843, 52844, 52845, 52846, 52847, 52848, 52849, 52850, 52851, 52852 were selected from the ORFs having the nucleotide sequence represented by SEQ ID NO: 4226.

The probes having the nucleotide sequence represented by SEQ ID NOS: 52853, 52854, 52855, 52856, 52857, 52858, 52859, 52860, 52861, 52862, 52863 were selected from the ORFs having the nucleotide sequence represented by SEQ ID NO: 4227.

The probes having the nucleotide sequence represented by SEQ ID NOS: 52864, 52865, 52866, 52867, 52868, 52869, 52870, 52871, 52872, 52873, 52874 were selected from the ORFs having the nucleotide sequence represented by SEQ ID NO: 4228.

The probes having the nucleotide sequence represented by SEQ ID NOS: 52875, 52876, 52877, 52878, 52879, 52880, 52881, 52882, 52883, 52884, 52885 were selected from the ORFs having the nucleotide sequence represented by SEQ ID NO: 4229.

The probes having the nucleotide sequence represented by SEQ ID NOS: 52886, 52887, 52888, 52889, 52890, 52891, 52892, 52893, 52894, 52895, 52896 were selected from the ORFs having the nucleotide sequence represented by SEQ ID NO: 4230.

The probes having the nucleotide sequence represented by SEQ ID NOS: 52897, 52898, 52899, 52900, 52901, 52902, 52903, 52904, 52905, 52906, 52907 were selected from the ORFs having the nucleotide sequence represented by SEQ ID NO: 4231.

The probes having the nucleotide sequence represented by SEQ ID NOS: 52908, 52909, 52910, 52911, 52912, 52913, 52914, 52915, 52916, 52917, 52918 were selected from the ORFs having the nucleotide sequence represented by SEQ ID NO: 4232.

The probes having the nucleotide sequence represented by SEQ ID NOS: 52919, 52920, 52921, 52922, 52923, 52924, 52925, 52926, 52927, 52928, 52929 were selected from the ORFs having the nucleotide sequence represented by SEQ ID NO: 4233.

The probes having the nucleotide sequence represented by SEQ ID NOS: 52930, 52931, 52932, 52933, 52934, 52935, 52936, 52937, 52938, 52939, 52940 were selected from the ORFs having the nucleotide sequence represented by SEQ ID NO: 4234.

The probes having the nucleotide sequence represented by SEQ ID NOS: 52941, 52942, 52943, 52944, 52945, 52946, 52947, 52948, 52949, 52950, 52951 were selected from the ORFs having the nucleotide sequence represented by SEQ ID NO: 4235.

The probes having the nucleotide sequence represented by SEQ ID NOS: 52952, 52953, 52954, 52955, 52956, 52957, 52958, 52959, 52960, 52961, 52962 were selected from the ORFs having the nucleotide sequence represented by SEQ ID NO: 4236.

The probes having the nucleotide sequence represented by SEQ ID NOS: 52963, 52964, 52965, 52966, 52967, 52968, 52969, 52970, 52971, 52972, 52973 were selected from the ORFs having the nucleotide sequence represented by SEQ ID NO: 4237.

The probes having the nucleotide sequence represented by SEQ ID NOS: 52974, 52975, 52976, 52977, 52978, 52979, 52980, 52981, 52982, 52983, 52984 were selected from the ORFs having the nucleotide sequence represented by SEQ ID NO: 4238.

The probes having the nucleotide sequence represented by SEQ ID NOS: 52985, 52986, 52987, 52988, 52989, 52990, 52991, 52992, 52993, 52994, 52995 were selected from the ORFs having the nucleotide sequence represented by SEQ ID NO: 4239.

The probes having the nucleotide sequence represented by SEQ ID NOS: 52996, 52997, 52998, 52999, 53000, 53001, 53002, 53003, 53004, 53005, 53006 were selected from the ORFs having the nucleotide sequence represented by SEQ ID NO: 4240.

The probes having the nucleotide sequence represented by SEQ ID NOS: 53007, 53008, 53009, 53010, 53011, 53012, 53013, 53014, 53015, 53016, 53017 were selected from the ORFs having the nucleotide sequence represented by SEQ ID NO: 4241.

The probes having the nucleotide sequence represented by SEQ ID NOS: 53018, 53019, 53020, 53021, 53022, 53023, 53024, 53025, 53026, 53027, 53028 were selected from the ORFs having the nucleotide sequence represented by SEQ ID NO: 4242.

The probes having the nucleotide sequence represented by SEQ ID NOS: 53029, 53030, 53031, 53032, 53033, 53034, 53035, 53036, 53037, 53038, 53039 were selected from the ORFs having the nucleotide sequence represented by SEQ ID NO: 4243.

The probes having the nucleotide sequence represented by SEQ ID NOS: 53040, 53041, 53042, 53043, 53044, 53045, 53046, 53047, 53048, 53049, 53050 were selected from the ORFs having the nucleotide sequence represented by SEQ ID NO: 4244.

The probes having the nucleotide sequence represented by SEQ ID NOS: 53051, 53052, 53053, 53054, 53055, 53056, 53057, 53058, 53059, 53060, 53061 were selected from the ORFs having the nucleotide sequence represented by SEQ ID NO: 4245.

The probes having the nucleotide sequence represented by SEQ ID NOS: 53062, 53063, 53064, 53065, 53066, 53067, 53068, 53069, 53070, 53071, 53072 were selected from the ORFs having the nucleotide sequence represented by SEQ ID NO: 4246.

The probes having the nucleotide sequence represented by SEQ ID NOS: 53073, 53074, 53075, 53076, 53077, 53078, 53079, 53080, 53081, 53082, 53083 were selected from the ORFs having the nucleotide sequence represented by SEQ ID NO: 4247.

The probes having the nucleotide sequence represented by SEQ ID NOS: 53084, 53085, 53086, 53087, 53088, 53089, 53090, 53091, 53092, 53093, 53094 were selected from the ORFs having the nucleotide sequence represented by SEQ ID NO: 4248.

The probes having the nucleotide sequence represented by SEQ ID NOS: 53095, 53096, 53097, 53098, 53099, 53100, 53101, 53102, 53103, 53104, 53105 were selected from the ORFs having the nucleotide sequence represented by SEQ ID NO: 4249.

The probes having the nucleotide sequence represented by SEQ ID NOS: 53106, 53107, 53108, 53109, 53110, 53111, 53112, 53113, 53114, 53115, 53116 were selected from the ORFs having the nucleotide sequence represented by SEQ ID NO: 4250.

The probes having the nucleotide sequence represented by SEQ ID NOS: 53117, 53118, 53119, 53120, 53121, 53122, 53123, 53124, 53125, 53126, 53127 were selected from the ORFs having the nucleotide sequence represented by SEQ ID NO: 4251.

The probes having the nucleotide sequence represented by SEQ ID NOS: 53128, 53129, 53130, 53131, 53132, 53133, 53134, 53135, 53136, 53137, 53138 were selected from the ORFs having the nucleotide sequence represented by SEQ ID NO: 4252.

The probes having the nucleotide sequence represented by SEQ ID NOS: 53139, 53140, 53141, 53142, 53143, 53144, 53145, 53146, 53147, 53148, 53149 were selected from the ORFs having the nucleotide sequence represented by SEQ ID NO: 4253.

The probes having the nucleotide sequence represented by SEQ ID NOS: 53150, 53151, 53152, 53153, 53154, 53155, 53156, 53157, 53158, 53159, 53160 were selected from the ORFs having the nucleotide sequence represented by SEQ ID NO: 4254.

The probes having the nucleotide sequence represented by SEQ ID NOS: 53161, 53162, 53163, 53164, 53165, 53166, 53167, 53168, 53169, 53170, 53171 were selected from the ORFs having the nucleotide sequence represented by SEQ ID NO: 4255.

The probes having the nucleotide sequence represented by SEQ ID NOS: 53172, 53173, 53174, 53175, 53176, 53177, 53178, 53179, 53180, 53181, 53182 were selected from the ORFs having the nucleotide sequence represented by SEQ ID NO: 4256.

The probes having the nucleotide sequence represented by SEQ ID NOS: 53183, 53184, 53185, 53186, 53187, 53188, 53189, 53190, 53191, 53192, 53193 were selected from the ORFs having the nucleotide sequence represented by SEQ ID NO: 4257.

The probes having the nucleotide sequence represented by SEQ ID NOS: 53194, 53195, 53196, 53197, 53198, 53199, 53200, 53201, 53202, 53203, 53204 were selected from the ORFs having the nucleotide sequence represented by SEQ ID NO: 4258.

The probes having the nucleotide sequence represented by SEQ ID NOS: 53205, 53206, 53207, 53208, 53209, 53210, 53211, 53212, 53213, 53214, 53215 were selected from the ORFs having the nucleotide sequence represented by SEQ ID NO: 4259.

The probes having the nucleotide sequence represented by SEQ ID NOS: 53216, 53217, 53218, 53219, 53220, 53221, 53222, 53223, 53224, 53225, 53226 were selected from the ORFs having the nucleotide sequence represented by SEQ ID NO: 4260.

The probes having the nucleotide sequence represented by SEQ ID NOS: 53227, 53228, 53229, 53230, 53231, 53232, 53233, 53234, 53235, 53236, 53237 were selected from the ORFs having the nucleotide sequence represented by SEQ ID NO: 4261.

The probes having the nucleotide sequence represented by SEQ ID NOS: 53238, 53239, 53240, 53241, 53242, 53243, 53244, 53245, 53246, 53247, 53248 were selected from the ORFs having the nucleotide sequence represented by SEQ ID NO: 4262.

The probes having the nucleotide sequence represented by SEQ ID NOS: 53249, 53250, 53251, 53252, 53253, 53254, 53255, 53256, 53257, 53258, 53259 were selected from the ORFs having the nucleotide sequence represented by SEQ ID NO: 4263.

The probes having the nucleotide sequence represented by SEQ ID NOS: 53260, 53261, 53262, 53263, 53264, 53265, 53266, 53267, 53268, 53269, 53270 were selected from the ORFs having the nucleotide sequence represented by SEQ ID NO: 4264.

The probes having the nucleotide sequence represented by SEQ ID NOS: 53271, 53272, 53273, 53274, 53275, 53276, 53277, 53278, 53279, 53280, 53281 were selected from the ORFs having the nucleotide sequence represented by SEQ ID NO: 4265.

The probes having the nucleotide sequence represented by SEQ ID NOS: 53282, 53283, 53284, 53285, 53286, 53287, 53288, 53289, 53290, 53291, 53292 were selected from the ORFs having the nucleotide sequence represented by SEQ ID NO: 4266.

The probes having the nucleotide sequence represented by SEQ ID NOS: 53293, 53294, 53295, 53296, 53297, 53298, 53299, 53300, 53301, 53302, 53303 were selected from the ORFs having the nucleotide sequence represented by SEQ ID NO: 4267.

The probes having the nucleotide sequence represented by SEQ ID NOS: 53304, 53305, 53306, 53307, 53308, 53309, 53310, 53311, 53312, 53313, 53314 were selected from the ORFs having the nucleotide sequence represented by SEQ ID NO: 4268.

The probes having the nucleotide sequence represented by SEQ ID NOS: 53315, 53316, 53317, 53318, 53319, 53320, 53321, 53322, 53323, 53324, 53325 were selected from the ORFs having the nucleotide sequence represented by SEQ ID NO: 4269.

The probes having the nucleotide sequence represented by SEQ ID NOS: 53326, 53327, 53328, 53329, 53330, 53331, 53332, 53333, 53334, 53335, 53336 were selected from the ORFs having the nucleotide sequence represented by SEQ ID NO: 4270.

The probes having the nucleotide sequence represented by SEQ ID NOS: 53337, 53338, 53339, 53340, 53341, 53342, 53343, 53344, 53345, 53346, 53347 were selected from the ORFs having the nucleotide sequence represented by SEQ ID NO: 4271.

The probes having the nucleotide sequence represented by SEQ ID NOS: 53348, 53349, 53350, 53351, 53352, 53353, 53354, 53355, 53356, 53357, 53358 were selected from the ORFs having the nucleotide sequence represented by SEQ ID NO: 4272.

The probes having the nucleotide sequence represented by SEQ ID NOS: 53359, 53360, 53361, 53362, 53363, 53364, 53365, 53366, 53367, 53368, 53369 were selected from the ORFs having the nucleotide sequence represented by SEQ ID NO: 4273.

The probes having the nucleotide sequence represented by SEQ ID NOS: 53370, 53371, 53372, 53373, 53374, 53375, 53376, 53377, 53378, 53379, 53380 were selected from the ORFs having the nucleotide sequence represented by SEQ ID NO: 4274.

The probes having the nucleotide sequence represented by SEQ ID NOS: 53381, 53382, 53383, 53384, 53385, 53386, 53387, 53388, 53389, 53390, 53391, 53392, 53393, 53394, 53395, 53396, 53397, 53398, 53399, 53400, 53401, 53402, 53403, 53404, 53405, 53406, 53407, 53408, 53409, 53410, 53411, 53412, 53413 were selected from the ORFs having the nucleotide sequence represented by SEQ ID NO: 4275.

The probes having the nucleotide sequence represented by SEQ ID NOS: 53414, 53415, 53416, 53417, 53418, 53419, 53420, 53421, 53422, 53423, 53424 were selected from the ORFs having the nucleotide sequence represented by SEQ ID NO: 4276.

The probes having the nucleotide sequence represented by SEQ ID NOS: 53425, 53426, 53427, 53428, 53429, 53430, 53431, 53432, 53433, 53434, 53435 were selected from the ORFs having the nucleotide sequence represented by SEQ ID NO: 4277.

The probes having the nucleotide sequence represented by SEQ ID NOS: 53436, 53437, 53438, 53439, 53440, 53441, 53442, 53443, 53444, 53445, 53446 were selected from the ORFs having the nucleotide sequence represented by SEQ ID NO: 4278.

The probes having the nucleotide sequence represented by SEQ ID NOS: 53447, 53448, 53449, 53450, 53451, 53452, 53453, 53454, 53455, 53456, 53457, 53458, 53459, 53460, 53461, 53462, 53463, 53464, 53465, 53466, 53467, 53468 were selected from the ORFs having the nucleotide sequence represented by SEQ ID NO: 4279.

The probes having the nucleotide sequence represented by SEQ ID NOS: 53469, 53470, 53471, 53472, 53473, 53474, 53475, 53476, 53477, 53478, 53479 were selected from the ORFs having the nucleotide sequence represented by SEQ ID NO: 4280.

The probes having the nucleotide sequence represented by SEQ ID NOS: 53480, 53481, 53482, 53483, 53484, 53485, 53486, 53487, 53488, 53489, 53490 were selected from the ORFs having the nucleotide sequence represented by SEQ ID NO: 4281.

The probes having the nucleotide sequence represented by SEQ ID NOS: 53491, 53492, 53493, 53494, 53495, 53496, 53497, 53498, 53499, 53500, 53501 were selected from the ORFs having the nucleotide sequence represented by SEQ ID NO: 4282.

The probes having the nucleotide sequence represented by SEQ ID NOS: 53502, 53503, 53504, 53505, 53506, 53507, 53508, 53509, 53510, 53511, 53512 were selected from the ORFs having the nucleotide sequence represented by SEQ ID NO: 4283.

The probes having the nucleotide sequence represented by SEQ ID NOS: 53513, 53514, 53515, 53516, 53517, 53518, 53519, 53520, 53521, 53522, 53523 were selected from the ORFs having the nucleotide sequence represented by SEQ ID NO: 4284.

The probes having the nucleotide sequence represented by SEQ ID NOS: 53524, 53525, 53526, 53527, 53528, 53529, 53530, 53531, 53532, 53533, 53534 were selected from the ORFs having the nucleotide sequence represented by SEQ ID NO: 4285.

The probes having the nucleotide sequence represented by SEQ ID NOS: 53535, 53536, 53537, 53538, 53539, 53540, 53541, 53542, 53543, 53544, 53545 were selected from the ORFs having the nucleotide sequence represented by SEQ ID NO: 4286.

The probes having the nucleotide sequence represented by SEQ ID NOS: 53546, 53547, 53548, 53549, 53550, 53551, 53552, 53553, 53554, 53555, 53556 were selected from the ORFs having the nucleotide sequence represented by SEQ ID NO: 4287.

The probes having the nucleotide sequence represented by SEQ ID NOS: 53557, 53558, 53559, 53560, 53561, 53562, 53563, 53564, 53565, 53566, 53567 were selected from the ORFs having the nucleotide sequence represented by SEQ ID NO: 4288.

The probes having the nucleotide sequence represented by SEQ ID NOS: 53568, 53569, 53570, 53571, 53572, 53573, 53574, 53575, 53576, 53577, 53578 were selected from the ORFs having the nucleotide sequence represented by SEQ ID NO: 4289.

The probes having the nucleotide sequence represented by SEQ ID NOS: 53579, 53580, 53581, 53582, 53583, 53584, 53585, 53586, 53587, 53588, 53589 were selected from the ORFs having the nucleotide sequence represented by SEQ ID NO: 4290.

The probes having the nucleotide sequence represented by SEQ ID NOS: 53590, 53591, 53592, 53593, 53594, 53595, 53596, 53597, 53598, 53599, 53600 were selected from the ORFs having the nucleotide sequence represented by SEQ ID NO: 4291.

The probes having the nucleotide sequence represented by SEQ ID NOS. 53601, 53602, 53603, 53604, 53605, 53606, 53607, 53608, 53609, 53610, 53611 were selected from the ORFs having the nucleotide sequence represented by SEQ ID NO: 4292.

The probes having the nucleotide sequence represented by SEQ ID NOS: 53612, 53613, 53614, 53615, 53616, 53617, 53618, 53619, 53620, 53621, 53622 were selected from the ORFs having the nucleotide sequence represented by SEQ ID NO: 4293.

The probes having the nucleotide sequence represented by SEQ ID NOS: 53623, 53624, 53625, 53626, 53627, 53628, 53629, 53630, 53631, 53632, 53633, 53634, 53635, 53636, 53637, 53638, 53639, 53640, 53641, 53642, 53643, 53644 were selected from the ORFs having the nucleotide sequence represented by SEQ ID NO: 4294.

The probes having the nucleotide sequence represented by SEQ ID NOS: 53645, 53646, 53647, 53648, 53649, 53650, 53651, 53652, 53653, 53654, 53655 were selected from the ORFs having the nucleotide sequence represented by SEQ ID NO: 4295.

The probes having the nucleotide sequence represented by SEQ ID NOS: 53656, 53657, 53658, 53659, 53660, 53661, 53662, 53663, 53664, 53665, 53666 were selected from the ORFs having the nucleotide sequence represented by SEQ ID NO: 4296.

The probes having the nucleotide sequence represented by SEQ ID NOS: 53667, 53668, 53669, 53670, 53671, 53672, 53673, 53674, 53675, 53676, 53677 were selected from the ORFs having the nucleotide sequence represented by SEQ ID NO: 4297.

The probes having the nucleotide sequence represented by SEQ ID NOS: 53678, 53679, 53680, 53681, 53682, 53683, 53684, 53685, 53686, 53687, 53688 were selected from the ORFs having the nucleotide sequence represented by SEQ ID NO: 4298.

The probes having the nucleotide sequence represented by SEQ ID NOS: 53689, 53690, 53691, 53692, 53693, 53694, 53695, 53696, 53697, 53698, 53699 were selected from the ORFs having the nucleotide sequence represented by SEQ ID NO: 4299.

The probes having the nucleotide sequence represented by SEQ ID NOS: 53700, 53701, 53702, 53703, 53704, 53705, 53706, 53707, 53708, 53709, 53710 were selected from the ORFs having the nucleotide sequence represented by SEQ ID NO: 4300.

The probes having the nucleotide sequence represented by SEQ ID NOS: 53711, 53712, 53713, 53714, 53715, 53716, 53717, 53718, 53719, 53720, 53721 were selected from the ORFs having the nucleotide sequence represented by SEQ ID NO: 4301.

The probes having the nucleotide sequence represented by SEQ ID NOS: 53722, 53723, 53724, 53725, 53726, 53727, 53728, 53729, 53730, 53731, 53732 were selected from the ORFs having the nucleotide sequence represented by SEQ ID NO: 4302.

The probes having the nucleotide sequence represented by SEQ ID NOS: 53733, 53734, 53735, 53736, 53737, 53738, 53739, 53740, 53741, 53742, 53743 were selected from the ORFs having the nucleotide sequence represented by SEQ ID NO: 4303.

The probes having the nucleotide sequence represented by SEQ ID NOS: 53744, 53745, 53746, 53747, 53748, 53749, 53750, 53751, 53752, 53753, 53754 were selected from the ORFs having the nucleotide sequence represented by SEQ ID NO: 4304.

The probes having the nucleotide sequence represented by SEQ ID NOS: 53755, 53756, 53757, 53758, 53759, 53760, 53761, 53762, 53763, 53764, 53765 were selected from the ORFs having the nucleotide sequence represented by SEQ ID NO: 4305.

The probes having the nucleotide sequence represented by SEQ ID NOS: 53766, 53767, 53768, 53769, 53770, 53771, 53772, 53773, 53774, 53775, 53776 were selected from the ORFs having the nucleotide sequence represented by SEQ ID NO: 4306.

The probes having the nucleotide sequence represented by SEQ ID NOS: 53777, 53778, 53779, 53780, 53781, 53782, 53783, 53784, 53785, 53786, 53787 were selected from the ORFs having the nucleotide sequence represented by SEQ ID NO: 4307.

The probes having the nucleotide sequence represented by SEQ ID NOS: 53788, 53789, 53790, 53791, 53792, 53793, 53794, 53795, 53796, 53797, 53798 were selected from the ORFs having the nucleotide sequence represented by SEQ ID NO: 4308.

The probes having the nucleotide sequence represented by SEQ ID NOS: 53799, 53800, 53801, 53802, 53803, 53804, 53805, 53806, 53807, 53808, 53809 were selected from the ORFs having the nucleotide sequence represented by SEQ ID NO: 4309.

The probes having the nucleotide sequence represented by SEQ ID NOS: 53810, 53811, 53812, 53813, 53814, 53815, 53816, 53817, 53818, 53819, 53820 were selected from the ORFs having the nucleotide sequence represented by SEQ ID NO: 4310.

The probes having the nucleotide sequence represented by SEQ ID NOS: 53821, 53822, 53823, 53824, 53825, 53826, 53827, 53828, 53829, 53830, 53831 were selected from the ORFs having the nucleotide sequence represented by SEQ ID NO: 4311.

The probes having the nucleotide sequence represented by SEQ ID NOS: 53832, 53833, 53834, 53835, 53836, 53837, 53838, 53839, 53840, 53841, 53842 were selected from the ORFs having the nucleotide sequence represented by SEQ ID NO: 4312.

The probes having the nucleotide sequence represented by SEQ ID NOS: 53843, 53844, 53845, 53846, 53847, 53848, 53849, 53850, 53851, 53852, 53853 were selected from the ORFs having the nucleotide sequence represented by SEQ ID NO: 4313.

The probes having the nucleotide sequence represented by SEQ ID NOS: 53854, 53855, 53856, 53857, 53858, 53859, 53860, 53861, 53862, 53863, 53864 were selected from the ORFs having the nucleotide sequence represented by SEQ ID NO: 4314.

The probes having the nucleotide sequence represented by SEQ ID NOS: 53865, 53866, 53867, 53868, 53869, 53870, 53871, 53872, 53873, 53874, 53875 were selected from the ORFs having the nucleotide sequence represented by SEQ ID NO: 4315.

The probes having the nucleotide sequence represented by SEQ ID NOS: 53876, 53877, 53878, 53879, 53880, 53881, 53882, 53883, 53884, 53885, 53886 were selected from the ORFs having the nucleotide sequence represented by SEQ ID NO: 4316.

The probes having the nucleotide sequence represented by SEQ ID NOS: 53887, 53888, 53889, 53890, 53891, 53892, 53893, 53894, 53895, 53896, 53897 were selected from the ORFs having the nucleotide sequence represented by SEQ ID NO: 4317.

The probes having the nucleotide sequence represented by SEQ ID NOS: 53898, 53899, 53900, 53901, 53902, 53903, 53904, 53905, 53906, 53907, 53908 were selected from the ORFs having the nucleotide sequence represented by SEQ ID NO: 4318.

The probes having the nucleotide sequence represented by SEQ ID NOS: 53909, 53910, 53911, 53912, 53913, 53914, 53915, 53916, 53917, 53918, 53919 were selected from the ORFs having the nucleotide sequence represented by SEQ ID NO: 4319.

The probes having the nucleotide sequence represented by SEQ ID NOS: 53920, 53921, 53922, 53923, 53924, 53925, 53926, 53927, 53928, 53929, 53930 were selected from the ORFs having the nucleotide sequence represented by SEQ ID NO: 4320.

The probes having the nucleotide sequence represented by SEQ ID NOS: 53931, 53932, 53933, 53934, 53935, 53936, 53937, 53938, 53939, 53940, 53941 were selected from the ORFs having the nucleotide sequence represented by SEQ ID NO: 4321.

The probes having the nucleotide sequence represented by SEQ ID NOS: 53942, 53943, 53944, 53945, 53946, 53947, 53948, 53949, 53950, 53951, 53952 were selected from the ORFs having the nucleotide sequence represented by SEQ ID NO: 4322.

The probes having the nucleotide sequence represented by SEQ ID NOS: 53953, 53954, 53955, 53956, 53957, 53958, 53959, 53960, 53961, 53962, 53963 were selected from the ORFs having the nucleotide sequence represented by SEQ ID NO: 4323.

The probes having the nucleotide sequence represented by SEQ ID NOS: 53964, 53965, 53966, 53967, 53968, 53969, 53970, 53971, 53972, 53973, 53974, 53975, 53976, 53977, 53978, 53979, 53980, 53981, 53982, 53983, 53984, 53985 were selected from the ORFs having the nucleotide sequence represented by SEQ ID NO: 4324.

The probes having the nucleotide sequence represented by SEQ ID NOS: 53986, 53987, 53988, 53989, 53990, 53991, 53992, 53993, 53994, 53995, 53996 were selected from the ORFs having the nucleotide sequence represented by SEQ ID NO: 4325.

The probes having the nucleotide sequence represented by SEQ ID NOS: 53997, 53998, 53999, 54000, 54001, 54002, 54003, 54004, 54005, 54006, 54007 were selected from the ORFs having the nucleotide sequence represented by SEQ ID NO: 4326.

The probes having the nucleotide sequence represented by SEQ ID NOS: 54008, 54009, 54010, 54011, 54012, 54013, 54014, 54015, 54016, 54017, 54018 were selected from the ORFs having the nucleotide sequence represented by SEQ ID NO: 4327.

The probes having the nucleotide sequence represented by SEQ ID NOS: 54019, 54020, 54021, 54022, 54023, 54024, 54025, 54026, 54027, 54028, 54029 were selected from the ORFs having the nucleotide sequence represented by SEQ ID NO: 4328.

The probes having the nucleotide sequence represented by SEQ ID NOS: 54030, 54031, 54032, 54033, 54034, 54035, 54036, 54037, 54038, 54039, 54040 were selected from the ORFs having the nucleotide sequence represented by SEQ ID NO: 4329.

The probes having the nucleotide sequence represented by SEQ ID NOS: 54041, 54042, 54043, 54044, 54045, 54046, 54047, 54048, 54049, 54050, 54051 were selected from the ORFs having the nucleotide sequence represented by SEQ ID NO: 4330.

The probes having the nucleotide sequence represented by SEQ ID NOS: 54052, 54053, 54054, 54055, 54056, 54057, 54058, 54059, 54060, 54061, 54062 were selected from the ORFs having the nucleotide sequence represented by SEQ ID NO: 4331.

The probes having the nucleotide sequence represented by SEQ ID NOS: 54063, 54064, 54065, 54066, 54067, 54068, 54069, 54070, 54071, 54072, 54073 were selected from the ORFs having the nucleotide sequence represented by SEQ ID NO: 4332.

The probes having the nucleotide sequence represented by SEQ ID NOS: 54074, 54075, 54076, 54077, 54078, 54079, 54080, 54081, 54082, 54083, 54084 were selected from the ORFs having the nucleotide sequence represented by SEQ ID NO: 4333.

The probes having the nucleotide sequence represented by SEQ ID NOS: 54085, 54086, 54087, 54088, 54089, 54090, 54091, 54092, 54093, 54094, 54095 were selected from the ORFs having the nucleotide sequence represented by SEQ ID NO: 4334.

The probes having the nucleotide sequence represented by SEQ ID NOS: 54096, 54097, 54098, 54099, 54100, 54101, 54102, 54103, 54104, 54105, 54106 were selected from the ORFs having the nucleotide sequence represented by SEQ ID NO: 4335.

The probes having the nucleotide sequence represented by SEQ ID NOS: 54107, 54108, 54109, 54110, 54111, 54112, 54113, 54114, 54115, 54116, 54117 were selected from the ORFs having the nucleotide sequence represented by SEQ ID NO: 4336.

The probes having the nucleotide sequence represented by SEQ ID NOS: 54118, 54119, 54120, 54121, 54122, 54123, 54124, 54125, 54126, 54127, 54128 were selected from the ORFs having the nucleotide sequence represented by SEQ ID NO: 4337.

The probes having the nucleotide sequence represented by SEQ ID NOS: 54129, 54130, 54131, 54132, 54133, 54134, 54135, 54136, 54137, 54138, 54139 were selected from the ORFs having the nucleotide sequence represented by SEQ ID NO: 4338.

The probes having the nucleotide sequence represented by SEQ ID NOS: 54140, 54141, 54142, 54143, 54144, 54145, 54146, 54147, 54148, 54149, 54150 were selected from the ORFs having the nucleotide sequence represented by SEQ ID NO: 4339.

The probes having the nucleotide sequence represented by SEQ ID NOS: 54151, 54152, 54153, 54154, 54155, 54156, 54157, 54158, 54159, 54160, 54161 were selected from the ORFs having the nucleotide sequence represented by SEQ ID NO: 4340.

The probes having the nucleotide sequence represented by SEQ ID NOS: 54162, 54163, 54164, 54165, 54166, 54167, 54168, 54169, 54170, 54171, 54172 were selected from the ORFs having the nucleotide sequence represented by SEQ ID NO: 4341.

The probes having the nucleotide sequence represented by SEQ ID NOS: 54173, 54174, 54175, 54176, 54177, 54178, 54179, 54180, 54181, 54182, 54183 were selected from the ORFs having the nucleotide sequence represented by SEQ ID NO: 4342.

The probes having the nucleotide sequence represented by SEQ ID NOS: 54184, 54185, 54186, 54187, 54188, 54189, 54190, 54191, 54192, 54193, 54194 were selected from the ORFs having the nucleotide sequence represented by SEQ ID NO: 4343.

The probes having the nucleotide sequence represented by SEQ ID NOS: 54195, 54196, 54197, 54198, 54199, 54200, 54201, 54202, 54203, 54204, 54205 were selected from the ORFs having the nucleotide sequence represented by SEQ ID NO: 4344.

The probes having the nucleotide sequence represented by SEQ ID NOS: 54206, 54207, 54208, 54209, 54210, 54211, 54212, 54213, 54214, 54215, 54216 were selected from the ORFs having the nucleotide sequence represented by SEQ ID NO: 4345.

The probes having the nucleotide sequence represented by SEQ ID NOS: 54217, 54218, 54219, 54220, 54221, 54222, 54223, 54224, 54225, 54226, 54227 were selected from the ORFs having the nucleotide sequence represented by SEQ ID NO: 4346.

The probes having the nucleotide sequence represented by SEQ ID NOS: 54228, 54229, 54230, 54231, 54232, 54233, 54234, 54235, 54236, 54237, 54238 were selected from the ORFs having the nucleotide sequence represented by SEQ ID NO: 4347.

The probes having the nucleotide sequence represented by SEQ ID NOS: 54239, 54240, 54241, 54242, 54243, 54244, 54245, 54246, 54247, 54248, 54249 were selected from the ORFs having the nucleotide sequence represented by SEQ ID NO: 4348.

The probes having the nucleotide sequence represented by SEQ ID NOS: 54250, 54251, 54252, 54253, 54254, 54255, 54256, 54257, 54258, 54259, 54260 were selected from the ORFs having the nucleotide sequence represented by SEQ ID NO: 4349.

The probes having the nucleotide sequence represented by SEQ ID NOS: 54261, 54262, 54263, 54264, 54265, 54266, 54267, 54268, 54269, 54270, 54271 were selected from the ORFs having the nucleotide sequence represented by SEQ ID NO: 4350.

The probes having the nucleotide sequence represented by SEQ ID NOS: 54272, 54273, 54274, 54275, 54276, 54277, 54278, 54279, 54280, 54281, 54282 were selected from the ORFs having the nucleotide sequence represented by SEQ ID NO: 4351.

The probes having the nucleotide sequence represented by SEQ ID NOS: 54283, 54284, 54285, 54286, 54287, 54288, 54289, 54290, 54291, 54292, 54293 were selected from the ORFs having the nucleotide sequence represented by SEQ ID NO: 4352.

The probes having the nucleotide sequence represented by SEQ ID NOS: 54294, 54295, 54296, 54297, 54298, 54299, 54300, 54301, 54302, 54303, 54304 were selected from the ORFs having the nucleotide sequence represented by SEQ ID NO: 4353.

The probes having the nucleotide sequence represented by SEQ ID NOS: 54305, 54306, 54307, 54308, 54309, 54310, 54311, 54312, 54313, 54314, 54315 were selected from the ORFs having the nucleotide sequence represented by SEQ ID NO: 4354.

The probes having the nucleotide sequence represented by SEQ ID NOS: 54316, 54317, 54318, 54319, 54320, 54321, 54322, 54323, 54324, 54325, 54326 were selected from the ORFs having the nucleotide sequence represented by SEQ ID NO: 4355.

The probes having the nucleotide sequence represented by SEQ ID NOS: 54327, 54328, 54329, 54330, 54331, 54332, 54333, 54334, 54335, 54336, 54337 were selected from the ORFs having the nucleotide sequence represented by SEQ ID NO: 4356.

The probes having the nucleotide sequence represented by SEQ ID NOS: 54338, 54339, 54340, 54341, 54342, 54343, 54344, 54345, 54346, 54347, 54348 were selected from the ORFs having the nucleotide sequence represented by SEQ ID NO: 4357.

The probes having the nucleotide sequence represented by SEQ ID NOS: 54349, 54350, 54351, 54352, 54353, 54354, 54355, 54356, 54357, 54358, 54359 were selected from the ORFs having the nucleotide sequence represented by SEQ ID NO: 4358.

The probes having the nucleotide sequence represented by SEQ ID NOS: 54360, 54361, 54362, 54363, 54364, 54365, 54366, 54367, 54368, 54369, 54370 were selected from the ORFs having the nucleotide sequence represented by SEQ ID NO: 4359.

The probes having the nucleotide sequence represented by SEQ ID NOS: 54371, 54372, 54373, 54374, 54375, 54376, 54377, 54378, 54379, 54380, 54381 were selected from the ORFs having the nucleotide sequence represented by SEQ ID NO: 4360.

The probes having the nucleotide sequence represented by SEQ ID NOS: 54382, 54383, 54384, 54385, 54386, 54387, 54388, 54389, 54390, 54391, 54392 were selected from the ORFs having the nucleotide sequence represented by SEQ ID NO: 4361.

The probes having the nucleotide sequence represented by SEQ ID NOS: 54393, 54394, 54395, 54396, 54397, 54398, 54399, 54400, 54401, 54402, 54403 were selected from the ORFs having the nucleotide sequence represented by SEQ ID NO: 4362.

The probes having the nucleotide sequence represented by SEQ ID NOS: 54404, 54405, 54406, 54407, 54408, 54409, 54410, 54411, 54412, 54413, 54414 were selected from the ORFs having the nucleotide sequence represented by SEQ ID NO: 4363.

The probes having the nucleotide sequence represented by SEQ ID NOS: 54415, 54416, 54417, 54418, 54419, 54420, 54421, 54422, 54423, 54424, 54425 were selected from the ORFs having the nucleotide sequence represented by SEQ ID NO: 4364.

The probes having the nucleotide sequence represented by SEQ ID NOS: 54426, 54427, 54428, 54429, 54430, 54431, 54432, 54433, 54434, 54435, 54436 were selected from the ORFs having the nucleotide sequence represented by SEQ ID NO: 4365.

The probes having the nucleotide sequence represented by SEQ ID NOS: 54437, 54438, 54439, 54440, 54441, 54442, 54443, 54444, 54445, 54446, 54447 were selected from the ORFs having the nucleotide sequence represented by SEQ ID NO: 4366.

The probes having the nucleotide sequence represented by SEQ ID NOS: 54448, 54449, 54450, 54451, 54452, 54453, 54454, 54455, 54456, 54457, 54458 were selected from the ORFs having the nucleotide sequence represented by SEQ ID NO: 4367.

The probes having the nucleotide sequence represented by SEQ ID NOS: 54459, 54460, 54461, 54462, 54463, 54464, 54465, 54466, 54467, 54468, 54469 were selected from the ORFs having the nucleotide sequence represented by SEQ ID NO: 4368.

The probes having the nucleotide sequence represented by SEQ ID NOS: 54470, 54471, 54472, 54473, 54474, 54475, 54476, 54477, 54478, 54479, 54480 were selected from the ORFs having the nucleotide sequence represented by SEQ ID NO: 4369.

The probes having the nucleotide sequence represented by SEQ ID NOS: 54481, 54482, 54483, 54484, 54485, 54486, 54487, 54488, 54489, 54490, 54491 were selected from the ORFs having the nucleotide sequence represented by SEQ ID NO: 4370.

The probes having the nucleotide sequence represented by SEQ ID NOS: 54492, 54493, 54494, 54495, 54496, 54497, 54498, 54499, 54500, 54501, 54502 were selected from the ORFs having the nucleotide sequence represented by SEQ ID NO: 4371.

The probes having the nucleotide sequence represented by SEQ ID NOS: 54503, 54504, 54505, 54506, 54507, 54508, 54509, 54510, 54511, 54512, 54513 were selected from the ORFs having the nucleotide sequence represented by SEQ ID NO: 4372.

The probes having the nucleotide sequence represented by SEQ ID NOS: 54514, 54515, 54516, 54517, 54518, 54519, 54520, 54521, 54522, 54523, 54524 were selected from the ORFs having the nucleotide sequence represented by SEQ ID NO: 4373.

The probes having the nucleotide sequence represented by SEQ ID NOS: 54525, 54526, 54527, 54528, 54529, 54530, 54531, 54532, 54533, 54534, 54535 were selected from the ORFs having the nucleotide sequence represented by SEQ ID NO: 4374.

The probes having the nucleotide sequence represented by SEQ ID NOS: 54536, 54537, 54538, 54539, 54540, 54541, 54542, 54543, 54544, 54545, 54546 were selected from the ORFs having the nucleotide sequence represented by SEQ ID NO: 4375.

The probes having the nucleotide sequence represented by SEQ ID NOS: 54547, 54548, 54549, 54550, 54551, 54552, 54553, 54554, 54555, 54556, 54557 were selected from the ORFs having the nucleotide sequence represented by SEQ ID NO: 4376.

The probes having the nucleotide sequence represented by SEQ ID NOS: 54558, 54559, 54560, 54561, 54562, 54563, 54564, 54565, 54566, 54567, 54568 were selected from the ORFs having the nucleotide sequence represented by SEQ ID NO: 4377.

The probes having the nucleotide sequence represented by SEQ ID NOS: 54569, 54570, 54571, 54572, 54573, 54574, 54575, 54576, 54577, 54578, 54579 were selected from the ORFs having the nucleotide sequence represented by SEQ ID NO: 4378.

The probes having the nucleotide sequence represented by SEQ ID NOS: 54580, 54581, 54582, 54583, 54584, 54585, 54586, 54587, 54588, 54589, 54590 were selected from the ORFs having the nucleotide sequence represented by SEQ ID NO: 4379.

The probes having the nucleotide sequence represented by SEQ ID NOS: 54591, 54592, 54593, 54594, 54595, 54596, 54597, 54598, 54599, 54600, 54601 were selected from the ORFs having the nucleotide sequence represented by SEQ ID NO: 4380.

The probes having the nucleotide sequence represented by SEQ ID NOS: 54602, 54603, 54604, 54605, 54606, 54607, 54608, 54609, 54610, 54611, 54612 were selected from the ORFs having the nucleotide sequence represented by SEQ ID NO: 4381.

The probes having the nucleotide sequence represented by SEQ ID NOS: 54613, 54614, 54615, 54616, 54617, 54618, 54619, 54620, 54621, 54622, 54623 were selected from the ORFs having the nucleotide sequence represented by SEQ ID NO: 4382.

The probes having the nucleotide sequence represented by SEQ ID NOS: 54624, 54625, 54626, 54627, 54628, 54629, 54630, 54631, 54632, 54633, 54634 were selected from the ORFs having the nucleotide sequence represented by SEQ ID NO: 4383.

The probes having the nucleotide sequence represented by SEQ ID NOS: 54635, 54636, 54637, 54638, 54639, 54640, 54641, 54642, 54643, 54644, 54645 were selected from the ORFs having the nucleotide sequence represented by SEQ ID NO: 4384.

The probes having the nucleotide sequence represented by SEQ ID NOS: 54646, 54647, 54648, 54649, 54650, 54651, 54652, 54653, 54654, 54655, 54656 were selected from the ORFs having the nucleotide sequence represented by SEQ ID NO: 4385.

The probes having the nucleotide sequence represented by SEQ ID NOS: 54657, 54658, 54659, 54660, 54661, 54662, 54663, 54664, 54665, 54666, 54667 were selected from the ORFs having the nucleotide sequence represented by SEQ ID NO: 4386.

The probes having the nucleotide sequence represented by SEQ ID NOS: 54668, 54669, 54670, 54671, 54672, 54673, 54674, 54675, 54676, 54677, 54678 were selected from the ORFs having the nucleotide sequence represented by SEQ ID NO: 4387.

The probes having the nucleotide sequence represented by SEQ ID NOS: 54679, 54680, 54681, 54682, 54683, 54684, 54685, 54686, 54687, 54688, 54689 were selected from the ORFs having the nucleotide sequence represented by SEQ ID NO: 4388.

The probes having the nucleotide sequence represented by SEQ ID NOS: 54690, 54691, 54692, 54693, 54694, 54695, 54696, 54697, 54698, 54699, 54700 were selected from the ORFs having the nucleotide sequence represented by SEQ ID NO: 4389.

The probes having the nucleotide sequence represented by SEQ ID NOS: 54701, 54702, 54703, 54704, 54705, 54706, 54707, 54708, 54709, 54710, 54711 were selected from the ORFs having the nucleotide sequence represented by SEQ ID NO: 4390.

The probes having the nucleotide sequence represented by SEQ ID NOS: 54712, 54713, 54714, 54715, 54716, 54717, 54718, 54719, 54720, 54721, 54722 were selected from the ORFs having the nucleotide sequence represented by SEQ ID NO: 4391.

The probes having the nucleotide sequence represented by SEQ ID NOS: 54723, 54724, 54725, 54726, 54727, 54728, 54729, 54730, 54731, 54732, 54733 were selected from the ORFs having the nucleotide sequence represented by SEQ ID NO: 4392.

The probes having the nucleotide sequence represented by SEQ ID NOS: 54734, 54735, 54736, 54737, 54738, 54739, 54740, 54741, 54742, 54743, 54744 were selected from the ORFs having the nucleotide sequence represented by SEQ ID NO: 4393.

The probes having the nucleotide sequence represented by SEQ ID NOS: 54745, 54746, 54747, 54748, 54749, 54750, 54751, 54752, 54753, 54754, 54755 were selected from the ORFs having the nucleotide sequence represented by SEQ ID NO: 4394.

The probes having the nucleotide sequence represented by SEQ ID NOS: 54756, 54757, 54758, 54759, 54760, 54761, 54762, 54763, 54764, 54765, 54766 were selected from the ORFs having the nucleotide sequence represented by SEQ ID NO: 4395.

The probes having the nucleotide sequence represented by SEQ ID NOS: 54767, 54768, 54769, 54770, 54771, 54772, 54773, 54774, 54775, 54776, 54777 were selected from the ORFs having the nucleotide sequence represented by SEQ ID NO: 4396.

The probes having the nucleotide sequence represented by SEQ ID NOS: 54778, 54779, 54780, 54781, 54782, 54783, 54784, 54785, 54786, 54787, 54788 were selected from the ORFs having the nucleotide sequence represented by SEQ ID NO: 4397.

The probes having the nucleotide sequence represented by SEQ ID NOS: 54789, 54790, 54791, 54792, 54793, 54794, 54795, 54796, 54797, 54798, 54799 were selected from the ORFs having the nucleotide sequence represented by SEQ ID NO: 4398.

The probes having the nucleotide sequence represented by SEQ ID NOS: 54800, 54801, 54802, 54803, 54804, 54805, 54806, 54807, 54808, 54809, 54810 were selected from the ORFs having the nucleotide sequence represented by SEQ ID NO: 4399.

The probes having the nucleotide sequence represented by SEQ ID NOS: 54811, 54812, 54813, 54814, 54815, 54816, 54817, 54818, 54819, 54820, 54821 were selected from the ORFs having the nucleotide sequence represented by SEQ ID NO: 4400.

The probes having the nucleotide sequence represented by SEQ ID NOS: 54822, 54823, 54824, 54825, 54826, 54827, 54828, 54829, 54830, 54831, 54832 were selected from the ORFs having the nucleotide sequence represented by SEQ ID NO: 4401.

The probes having the nucleotide sequence represented by SEQ ID NOS: 54833, 54834, 54835, 54836, 54837, 54838, 54839, 54840, 54841, 54842, 54843 were selected from the ORFs having the nucleotide sequence represented by SEQ ID NO: 4402.

The probes having the nucleotide sequence represented by SEQ ID NOS: 54844, 54845, 54846, 54847, 54848, 54849, 54850, 54851, 54852, 54853, 54854 were selected from the ORFs having the nucleotide sequence represented by SEQ ID NO: 4403.

The probes having the nucleotide sequence represented by SEQ ID NOS: 54855, 54856, 54857, 54858, 54859, 54860, 54861, 54862, 54863, 54864, 54865 were selected from the ORFs having the nucleotide sequence represented by SEQ ID NO: 4404.

The probes having the nucleotide sequence represented by SEQ ID NOS: 54866, 54867, 54868, 54869, 54870, 54871, 54872, 54873, 54874, 54875, 54876 were selected from the ORFs having the nucleotide sequence represented by SEQ ID NO: 4405.

The probes having the nucleotide sequence represented by SEQ ID NOS: 54877, 54878, 54879, 54880, 54881, 54882, 54883, 54884, 54885, 54886, 54887, 54888, 54889, 54890, 54891, 54892, 54893, 54894, 54895, 54896, 54897, 54898 were selected from the ORFs having the nucleotide sequence represented by SEQ ID NO: 4406.

The probes having the nucleotide sequence represented by SEQ ID NOS: 54899, 54900, 54901, 54902, 54903, 54904, 54905, 54906, 54907, 54908, 54909 were selected from the ORFs having the nucleotide sequence represented by SEQ ID NO: 4407.

The probes having the nucleotide sequence represented by SEQ ID NOS: 54910, 54911, 54912, 54913, 54914, 54915, 54916, 54917, 54918, 54919, 54920 were selected from the ORFs having the nucleotide sequence represented by SEQ ID NO: 4408.

The probes having the nucleotide sequence represented by SEQ ID NOS: 54921, 54922, 54923, 54924, 54925, 54926, 54927, 54928, 54929, 54930, 54931 were selected from the ORFs having the nucleotide sequence represented by SEQ ID NO: 4409.

The probes having the nucleotide sequence represented by SEQ ID NOS: 54932, 54933, 54934, 54935, 54936, 54937, 54938, 54939, 54940, 54941, 54942 were selected from the ORFs having the nucleotide sequence represented by SEQ ID NO: 4410.

The probes having the nucleotide sequence represented by SEQ ID NOS: 54943, 54944, 54945, 54946, 54947, 54948, 54949, 54950, 54951, 54952, 54953 were selected from the ORFs having the nucleotide sequence represented by SEQ ID NO: 4411.

The probes having the nucleotide sequence represented by SEQ ID NOS: 54954, 54955, 54956, 54957, 54958, 54959, 54960, 54961, 54962, 54963, 54964 were selected from the ORFs having the nucleotide sequence represented by SEQ ID NO: 4412.

The probes having the nucleotide sequence represented by SEQ ID NOS: 54965, 54966, 54967, 54968, 54969, 54970, 54971, 54972, 54973, 54974, 54975 were selected from the ORFs having the nucleotide sequence represented by SEQ ID NO: 4413.

The probes having the nucleotide sequence represented by SEQ ID NOS: 54976, 54977, 54978, 54979, 54980, 54981, 54982, 54983, 54984, 54985, 54986 were selected from the ORFs having the nucleotide sequence represented by SEQ ID NO: 4414.

The probes having the nucleotide sequence represented by SEQ ID NOS: 54987, 54988, 54989, 54990, 54991, 54992, 54993, 54994, 54995, 54996, 54997 were selected from the ORFs having the nucleotide sequence represented by SEQ ID NO: 4415.

The probes having the nucleotide sequence represented by SEQ ID NOS: 54998, 54999, 55000, 55001, 55002, 55003, 55004, 55005, 55006, 55007, 55008 were selected from the ORFs having the nucleotide sequence represented by SEQ ID NO: 4416.

The probes having the nucleotide sequence represented by SEQ ID NOS: 55009, 55010, 55011, 55012, 55013, 55014, 55015, 55016, 55017, 55018, 55019 were selected from the ORFs having the nucleotide sequence represented by SEQ ID NO: 4417.

The probes having the nucleotide sequence represented by SEQ ID NOS: 55020, 55021, 55022, 55023, 55024, 55025, 55026, 55027, 55028, 55029, 55030 were selected from the ORFs having the nucleotide sequence represented by SEQ ID NO: 4418.

The probes having the nucleotide sequence represented by SEQ ID NOS: 55031, 55032, 55033, 55034, 55035, 55036, 55037, 55038, 55039, 55040, 55041, 55042, 55043, 55044, 55045, 55046, 55047, 55048, 55049, 55050, 55051, 55052 were selected from the ORFs having the nucleotide sequence represented by SEQ ID NO: 4419.

The probes having the nucleotide sequence represented by SEQ ID NOS: 55053, 55054, 55055, 55056, 55057, 55058, 55059, 55060, 55061, 55062, 55063 were selected from the ORFs having the nucleotide sequence represented by SEQ ID NO: 4420.

The probes having the nucleotide sequence represented by SEQ ID NOS: 55064, 55065, 55066, 55067, 55068, 55069, 55070, 55071, 55072, 55073, 55074 were selected from the ORFs having the nucleotide sequence represented by SEQ ID NO: 4421.

The probes having the nucleotide sequence represented by SEQ ID NOS: 55075, 55076, 55077, 55078, 55079, 55080, 55081, 55082, 55083, 55084, 55085 were selected from the ORFs having the nucleotide sequence represented by SEQ ID NO: 4422.

The probes having the nucleotide sequence represented by SEQ ID NOS: 55086, 55087, 55088, 55089, 55090, 55091, 55092, 55093, 55094 were selected from the ORFs having the nucleotide sequence represented by SEQ ID NO: 4423.

The probes having the nucleotide sequence represented by SEQ ID NOS: 55095, 55096, 55097, 55098, 55099, 55100, 55101, 55102, 55103, 55104, 55105 were selected from the ORFs having the nucleotide sequence represented by SEQ ID NO: 4424.

The probes having the nucleotide sequence represented by SEQ ID NOS: 55106, 55107, 55108, 55109, 55110, 55111, 55112, 55113, 55114, 55115, 55116 were selected from the ORFs having the nucleotide sequence represented by SEQ ID NO: 4425.

The probes having the nucleotide sequence represented by SEQ ID NOS: 55117, 55118, 55119, 55120, 55121, 55122, 55123, 55124, 55125, 55126, 55127 were selected from the ORFs having the nucleotide sequence represented by SEQ ID NO: 4426.

The probes having the nucleotide sequence represented by SEQ ID NOS: 55128, 55129, 55130, 55131, 55132, 55133, 55134, 55135, 55136, 55137, 55138 were selected from the ORFs having the nucleotide sequence represented by SEQ ID NO: 4427.

The probes having the nucleotide sequence represented by SEQ ID NOS: 55139, 55140, 55141, 55142, 55143, 55144, 55145, 55146, 55147, 55148, 55149 were selected from the ORFs having the nucleotide sequence represented by SEQ ID NO: 4428.

The probes having the nucleotide sequence represented by SEQ ID NOS: 55150, 55151, 55152, 55153, 55154, 55155, 55156, 55157, 55158, 55159, 55160 were selected from the ORFs having the nucleotide sequence represented by SEQ ID NO: 4429.

The probes having the nucleotide sequence represented by SEQ ID NOS: 55161, 55162, 55163, 55164, 55165, 55166, 55167, 55168, 55169, 55170, 55171 were selected from the ORFs having the nucleotide sequence represented by SEQ ID NO: 4430.

The probes having the nucleotide sequence represented by SEQ ID NOS: 55172, 55173, 55174, 55175, 55176, 55177, 55178, 55179, 55180, 55181, 55182 were selected from the ORFs having the nucleotide sequence represented by SEQ ID NO: 4431.

The probes having the nucleotide sequence represented by SEQ ID NOS: 55183, 55184, 55185, 55186, 55187, 55188, 55189, 55190, 55191, 55192, 55193 were selected from the ORFs having the nucleotide sequence represented by SEQ ID NO: 4432.

The probes having the nucleotide sequence represented by SEQ ID NOS: 55194, 55195, 55196, 55197, 55198, 55199, 55200, 55201, 55202, 55203, 55204 were selected from the ORFs having the nucleotide sequence represented by SEQ ID NO: 4433.

The probes having the nucleotide sequence represented by SEQ ID NOS: 55205, 55206, 55207, 55208, 55209, 55210, 55211, 55212, 55213, 55214, 55215 were selected from the ORFs having the nucleotide sequence represented by SEQ ID NO: 4434.

The probes having the nucleotide sequence represented by SEQ ID NOS: 55216, 55217, 55218, 55219, 55220, 55221, 55222, 55223, 55224, 55225, 55226 were selected from the ORFs having the nucleotide sequence represented by SEQ ID NO: 4435.

The probes having the nucleotide sequence represented by SEQ ID NOS: 55227, 55228, 55229, 55230, 55231, 55232, 55233, 55234, 55235, 55236, 55237 were selected from the ORFs having the nucleotide sequence represented by SEQ ID NO: 4436.

The probes having the nucleotide sequence represented by SEQ ID NOS: 55238, 55239, 55240, 55241, 55242, 55243, 55244, 55245, 55246, 55247, 55248 were selected from the ORFs having the nucleotide sequence represented by SEQ ID NO: 4437.

The probes having the nucleotide sequence represented by SEQ ID NOS: 55249, 55250, 55251, 55252, 55253, 55254, 55255, 55256, 55257, 55258, 55259 were selected from the ORFs having the nucleotide sequence represented by SEQ ID NO: 4438.

The probes having the nucleotide sequence represented by SEQ ID NOS: 55260, 55261, 55262, 55263, 55264, 55265, 55266, 55267, 55268, 55269, 55270 were selected from the ORFs having the nucleotide sequence represented by SEQ ID NO: 4439.

The probes having the nucleotide sequence represented by SEQ ID NOS: 55271, 55272, 55273, 55274, 55275, 55276, 55277, 55278, 55279, 55280, 55281 were selected from the ORFs having the nucleotide sequence represented by SEQ ID NO: 4440.

The probes having the nucleotide sequence represented by SEQ ID NOS: 55282, 55283, 55284, 55285, 55286, 55287, 55288, 55289, 55290, 55291, 55292 were selected from the ORFs having the nucleotide sequence represented by SEQ ID NO: 4441.

The probes having the nucleotide sequence represented by SEQ ID NOS: 55293, 55294, 55295, 55296, 55297, 55298, 55299, 55300, 55301, 55302, 55303 were selected from the ORFs having the nucleotide sequence represented by SEQ ID NO: 4442.

The probes having the nucleotide sequence represented by SEQ ID NOS: 55304, 55305, 55306, 55307, 55308, 55309, 55310, 55311, 55312, 55313, 55314 were selected from the ORFs having the nucleotide sequence represented by SEQ ID NO: 4443.

The probes having the nucleotide sequence represented by SEQ ID NOS: 55315, 55316, 55317, 55318, 55319, 55320, 55321, 55322, 55323, 55324, 55325 were selected from the ORFs having the nucleotide sequence represented by SEQ ID NO: 4444.

The probes having the nucleotide sequence represented by SEQ ID NOS: 55326, 55327, 55328, 55329, 55330, 55.331, 55332, 55333, 55334, 55335, 55336 were selected from the ORFs having the nucleotide sequence represented by SEQ ID NO: 4445.

The probes having the nucleotide sequence represented by SEQ ID NOS: 55337, 55338, 55339, 55340, 55341, 55342, 55343, 55344, 55345, 55346, 55347 were selected from the ORFs having the nucleotide sequence represented by SEQ ID NO: 4446.

The probes having the nucleotide sequence represented by SEQ ID NOS: 55348, 55349, 55350, 55351, 55352, 55353, 55354, 55355, 55356, 55357, 55358 were selected from the ORFs having the nucleotide sequence represented by SEQ ID NO: 4447.

The probes having the nucleotide sequence represented by SEQ ID NOS: 55359, 55360, 55361, 55362, 55363, 55364, 55365, 55366, 55367, 55368, 55369 were selected from the ORFs having the nucleotide sequence represented by SEQ ID NO: 4448.

The probes having the nucleotide sequence represented by SEQ ID NOS: 55370, 55371, 55372, 55373, 55374, 55375, 55376, 55377, 55378, 55379, 55380 were selected from the ORFs having the nucleotide sequence represented by SEQ ID NO: 4449.

The probes having the nucleotide sequence represented by SEQ ID NOS: 55381, 55382, 55383, 55384, 55385, 55386, 55387, 55388, 55389, 55390, 55391 were selected from the ORFs having the nucleotide sequence represented by SEQ ID NO: 4450.

The probes having the nucleotide sequence represented by SEQ ID NOS: 55392, 55393, 55394, 55395, 55396, 55397, 55398, 55399, 55400, 55401, 55402 were selected from the ORFs having the nucleotide sequence represented by SEQ ID NO: 4451.

The probes having the nucleotide sequence represented by SEQ ID NOS: 55403, 55404, 55405, 55406, 55407, 55408, 55409, 55410, 55411, 55412, 55413 were selected from the ORFs having the nucleotide sequence represented by SEQ ID NO: 4452.

The probes having the nucleotide sequence represented by SEQ ID NOS: 55414, 55415, 55416, 55417, 55418, 55419, 55420, 55421, 55422, 55423,.55424 were selected from the ORFs having the nucleotide sequence represented by SEQ ID NO: 4453.

The probes having the nucleotide sequence represented by SEQ ID NOS: 55425, 55426, 55427, 55428, 55429, 55430, 55431, 55432, 55433, 55434, 55435 were selected from the ORFs having the nucleotide sequence represented by SEQ ID NO: 4454.

The probes having the nucleotide sequence represented by SEQ ID NOS: 55436, 55437, 55438, 55439, 55440, 55441, 55442, 55443, 55444, 55445, 55446 were selected from the ORFs having the nucleotide sequence represented by SEQ ID NO: 4455.

The probes having the nucleotide sequence represented by SEQ ID NOS: 55447, 55448, 55449, 55450, 55451, 55452, 55453, 55454, 55455, 55456, 55457 were selected from the ORFs having the nucleotide sequence represented by SEQ ID NO: 4456.

The probes having the nucleotide sequence represented by SEQ ID NOS: 55458, 55459, 55460, 55461, 55462, 55463, 55464, 55465, 55466, 55467, 55468 were selected from the ORFs having the nucleotide sequence represented by SEQ ID NO: 4457.

The probes having the nucleotide sequence represented by SEQ ID NOS: 55469, 55470, 55471, 55472, 55473, 55474, 55475, 55476, 55477, 55478, 55479 were selected from the ORFs having the nucleotide sequence represented by SEQ ID NO: 4458.

The probes having the nucleotide sequence represented by SEQ ID NOS: 55480,. 55481, 55482, 55483, 55484, 55485, 55486, 55487, 55488, 55489, 55490 were selected from the ORFs having the nucleotide sequence represented by SEQ ID NO: 4459.

The probes having the nucleotide sequence represented by SEQ ID NOS: 55491, 55492, 55493, 55494, 55495, 55496, 55497, 55498, 55499, 55500, 55501 were selected from the ORFs having the nucleotide sequence represented by SEQ ID NO: 4460.

The probes having the nucleotide sequence represented by SEQ ID NOS: 55502, 55503, 55504, 55505, 55506, 55507, 55508, 55509, 555110, 55511, 55512 were selected from the ORFs having the nucleotide sequence represented by SEQ ID NO: 4461.

The probes having the nucleotide sequence represented by SEQ ID NOS: 55513, 55514, 55515, 55516, 55517, 55518, 55519, 55520, 55521, 55522, 55523 were selected from the ORFs having the nucleotide sequence represented by SEQ ID NO: 4462.

The probes having the nucleotide sequence represented by SEQ ID NOS: 55524, 55525, 55526, 55527, 55528, 55529, 55530, 55531, 55532, 55533, 55534 were selected from the ORFs having the nucleotide sequence represented by SEQ ID NO: 4463.

The probes having the nucleotide sequence represented by SEQ ID NOS: 55535, 55536, 55537, 55538, 55539, 55540, 55541, 55542, 55543, 55544, 55545 were selected from the ORFs having the nucleotide sequence represented by SEQ ID NO: 4464.

The probes having the nucleotide sequence represented by SEQ ID NOS: 55546, 55547, 55548, 55549, 55550, 55551, 55552, 55553, 55554, 55555, 55556 were selected from the ORFs having the nucleotide sequence represented by SEQ ID NO: 4465.

The probes having the nucleotide sequence represented by SEQ ID NOS: 55557, 55558, 55559, 55560, 55561, 55562, 55563, 55564, 55565, 55566, 55567 were selected from the ORFs having the nucleotide sequence represented by SEQ ID NO: 4466.

The probes having the nucleotide sequence represented by SEQ ID NOS: 55568, 55569, 55570, 55571, 55572, 55573, 55574, 55575, 55576, 55577, 55578 were selected from the ORFs having the nucleotide sequence represented by SEQ ID NO: 4467.

The probes having the nucleotide sequence represented by SEQ ID NOS: 55579, 55580, 55581, 55582, 55583, 55584, 55585, 55586, 55587, 55588, 55589 were selected from the ORFs having the nucleotide sequence represented by SEQ ID NO: 4468.

The probes having the nucleotide sequence represented by SEQ ID NOS: 55590, 55591, 55592, 55593, 55594, 55595, 55596, 55597, 55598, 55599, 55600 were selected from the ORFs having the nucleotide sequence represented by SEQ ID NO: 4469.

The probes having the nucleotide sequence represented by SEQ ID NOS: 55601, 55602, 55603, 55604, 55605, 55606, 55607, 55608, 55609, 55610, 55611 were selected from the ORFs having the nucleotide sequence represented by SEQ ID NO: 4470.

The probes having the nucleotide sequence represented by SEQ ID NOS: 55612, 55613, 55614, 55615, 55616, 55617, 55618, 55619, 55620, 55621, 55622 were selected from the ORFs having the nucleotide sequence represented by SEQ ID NO: 4471.

The probes having the nucleotide sequence represented by SEQ ID NOS: 55623, 55624, 55625, 55626, 55627, 55628, 55629, 55630, 55631, 55632, 55633 were selected from the ORFs having the nucleotide sequence represented by SEQ ID NO: 4472.

The probes having the nucleotide sequence represented by SEQ ID NOS: 55634, 55635, 55636, 55637, 55638, 55639, 55640, 55641, 55642, 55643, 55644 were selected from the ORFs having the nucleotide sequence represented by SEQ ID NO: 4473.

The probes having the nucleotide sequence represented by SEQ ID NOS: 55645, 55646, 55647, 55648, 55649, 55650, 55651, 55652, 55653, 55654, 55655, 55656, 55657, 55658, 55659, 55660, 55661, 55662, 55663, 55664, 55665, 55666 were selected from the ORFs having the nucleotide sequence represented by SEQ ID NO: 4474.

The probes having the nucleotide sequence represented by SEQ ID NOS: 55667, 55668, 55669, 55670, 55671, 55672, 55673, 55674, 55675, 55676, 55677 were selected from the ORFs having the nucleotide sequence represented by SEQ ID NO: 4475.

The probes having the nucleotide sequence represented by SEQ ID NOS: 55678, 55679, 55680, 55681, 55682, 55683, 55684, 55685, 55686, 55687, 55688 were selected from the ORFs having the nucleotide sequence represented by SEQ ID NO: 4476.

The probes having the nucleotide sequence represented by SEQ ID NOS: 55689, 55690, 55691, 55692, 55693, 55694, 55695, 55696, 55697, 55698, 55699 were selected from the ORFs having the nucleotide sequence represented by SEQ ID NO: 4477.

The probes having the nucleotide sequence represented by SEQ ID NOS: 55700, 55701, 55702, 55703, 55704, 55705, 55706, 55707, 55708, 55709, 55710 were selected from the ORFs having the nucleotide sequence represented by SEQ ID NO: 4478.

The probes having the nucleotide sequence represented by SEQ ID NOS: 55711, 55712, 55713, 55714, 55715, 55716, 55717, 55118, 55719, 55720, 55721 were selected from the ORFs having the nucleotide sequence represented by SEQ ID NO: 4479.

The probes having the nucleotide sequence represented by SEQ ID NOS: 55722, 55723, 55724, 55725, 55726, 55727, 55728, 55729, 55730, 55731, 55732 were selected from the ORFs having the nucleotide sequence represented by SEQ ID NO: 4480.

The probes having the nucleotide sequence represented by SEQ ID NOS: 55733, 55734, 55735, 55736, 55737, 55738, 55739, 55740, 55741, 55742, 55743 were selected from the ORFs having the nucleotide sequence represented by SEQ ID NO: 4481.

The probes having the nucleotide sequence represented by SEQ ID NOS: 55744, 55745, 55746, 55747, 55748, 55749, 55750, 55751, 55752, 55753, 55754 were selected from the ORFs having the nucleotide sequence represented by SEQ ID NO: 4482.

The probes having the nucleotide sequence represented by SEQ ID NOS: 55755, 55756, 55757, 55758, 55759, 55760, 55761, 55762, 55763, 55764, 55765 were selected from the ORFs having the nucleotide sequence represented by SEQ ID NO: 4483.

The probes having the nucleotide sequence represented by SEQ ID NOS: 55766, 55767, 55768, 55769, 55770, 55771, 55772, 55773, 55774, 55775, 55776 were selected from the ORFs having the nucleotide sequence represented by SEQ ID NO: 4484.

The probes having the nucleotide sequence represented by SEQ ID NOS: 55777, 55778, 55779, 55780, 55781, 55782, 55783, 55784, 55785, 55786, 55787 were selected from the ORFs having the nucleotide sequence represented by SEQ ID NO: 4485.

The probes having the nucleotide sequence represented by SEQ ID NOS: 55788, 55789, 55790, 55791, 55792, 55793, 55794, 55795, 55796, 55797, 55798 were selected from the ORFs having the nucleotide sequence represented by SEQ ID NO: 4486.

The probes having the nucleotide sequence represented by SEQ ID NOS: 55799, 55800, 55801, 55802, 55803, 55804, 55805, 55806, 55807, 55808, 55809 were selected from the ORFs having the nucleotide sequence represented by SEQ ID NO: 4487.

The probes having the nucleotide sequence represented by SEQ ID NOS: 55810, 55811, 55812, 55813, 55814, 55815, 55816, 55817, 55818, 55819, 55820 were selected from the ORFs having the nucleotide sequence represented by SEQ ID NO: 4488.

The probes having the nucleotide sequence represented by SEQ ID NOS: 55821, 55822, 55823, 55824, 55825, 55826, 55827, 55828, 55829, 55830, 55831 were selected from the ORFs having the nucleotide sequence represented by SEQ ID NO: 4489.

The probes having the nucleotide sequence represented by SEQ ID NOS: 55832, 55833, 55834, 55835, 55836, 55837, 55838, 55839, 55840, 55841, 55842 were selected from the ORFs having the nucleotide sequence represented by SEQ ID NO: 4490.

The probes having the nucleotide sequence represented by SEQ ID NOS: 55843, 55844, 55845, 55846, 55847, 55848, 55849, 55850, 55851, 55852, 55853 were selected from the ORFs having the nucleotide sequence represented by SEQ ID NO: 4491.

The probes having the nucleotide sequence represented by SEQ ID NOS: 55854, 55855, 55856, 55857, 55858, 55859, 55860, 55861, 55862, 55863, 55864 were selected from the ORFs having the nucleotide sequence represented by SEQ ID NO: 4492.

The probes having the nucleotide sequence represented by SEQ ID NOS: 55865, 55866, 55867, 55868, 55869, 55870, 55871, 55872, 55873, 55874, 55875 were selected from the ORFs having the nucleotide sequence represented by SEQ ID NO: 4493.

The probes having the nucleotide sequence represented by SEQ ID NOS: 55876, 55877, 55878, 55879, 55880, 55881, 55882, 55883, 55884, 55885, 55886 were selected from the ORFs having the nucleotide sequence represented by SEQ ID NO: 4494.

The probes having the nucleotide sequence represented by SEQ ID NOS: 55887, 55888, 55889, 55890, 55891, 55892, 55893, 55894, 55895, 55896, 55897 were selected from the ORFs having the nucleotide sequence represented by SEQ ID NO: 4495.

The probes having the nucleotide sequence represented by SEQ ID NOS: 55898, 55899, 55900, 55901, 55902, 55903, 55904, 55905, 55906, 55907, 55908 were selected from the ORFs having the nucleotide sequence represented by SEQ ID NO: 4496.

The probes having the nucleotide sequence represented by SEQ ID NOS: 55909, 55910, 55911, 55912, 55913, 55914, 55915, 55916, 55917, 55918, 55919 were selected from the ORFs having the nucleotide sequence represented by SEQ ID NO: 4497.

The probes having the nucleotide sequence represented by SEQ ID NOS: 55920, 55921, 55922, 55923, 55924, 55925, 55926, 55927, 55928, 55929, 55930 were selected from the ORFs having the nucleotide sequence represented by SEQ ID NO: 4498.

The probes having the nucleotide sequence represented by SEQ ID NOS: 55931, 55932, 55933, 55934, 55935, 55936, 55937, 55938, 55939, 55940, 55941 were selected from the ORFs having the nucleotide sequence represented by SEQ ID NO: 4499.

The probes having the nucleotide sequence represented by SEQ ID NOS: 55942, 55943, 55944, 55945, 55946, 55947, 55948, 55949, 55950, 55951, 55952 were selected from the ORFs having the nucleotide sequence represented by SEQ ID NO: 4500.

The probes having the nucleotide sequence represented by SEQ ID NOS: 55953, 55954, 55955, 55956, 55957, 55958, 55959, 55960, 55961, 55962, 55963 were selected from the ORFs having the nucleotide sequence represented by SEQ ID NO: 4501.

The probes having the nucleotide sequence represented by SEQ ID NOS: 55964, 55965, 55966, 55967, 55968, 55969, 55970, 55971, 55972, 55973, 55974 were selected from the ORFs having the nucleotide sequence represented by SEQ ID NO: 4502.

The probes having the nucleotide sequence represented by SEQ ID NOS: 55975, 55976, 55977, 55978, 55979, 55980, 55981, 55982, 55983, 55984, 55985 were selected from the ORFs having the nucleotide sequence represented by SEQ ID NO: 4503.

The probes having the nucleotide sequence represented by SEQ ID NOS: 55986, 55987, 55988, 55989, 55990, 55991, 55992, 55993, 55994, 55995, 55996 were selected from the ORFs having the nucleotide sequence represented by SEQ ID NO: 4504.

The probes having the nucleotide sequence represented by SEQ ID NOS: 55997, 55998, 55999, 56000, 56001, 56002, 56003, 56004, 56005, 56006, 56007 were selected from the ORFs having the nucleotide sequence represented by SEQ ID NO: 4505.

The probes having the nucleotide sequence represented by SEQ ID NOS: 56008, 56009, 56010, 56011, 56012, 56013, 56014, 56015, 56016, 56017, 56018 were selected from the ORFs having the nucleotide sequence represented by SEQ ID NO: 4506.

The probes having the nucleotide sequence represented by SEQ ID NOS: 56019, 56020, 56021, 56022, 56023, 56024, 56025, 56026, 56027, 56028, 56029 were selected from the ORFs having the nucleotide sequence represented by SEQ ID NO: 4507.

The probes having the nucleotide sequence represented by SEQ ID NOS: 56030, 56031, 56032, 56033, 56034, 56035, 56036, 56037, 56038, 56039, 56040 were selected from the ORFs having the nucleotide sequence represented by SEQ ID NO: 4508.

The probes having the nucleotide sequence represented by SEQ ID NOS: 56041, 56042, 56043, 56044, 56045, 56046, 56047, 56048, 56049, 56050, 56051 were selected from the ORFs having the nucleotide sequence represented by SEQ ID NO: 4509.

The probes having the nucleotide sequence represented by SEQ ID NOS: 56052, 56053, 56054, 56055, 56056, 56057, 56058, 56059, 56060, 56061, 56062 were selected from the ORFs having the nucleotide sequence represented by SEQ ID NO: 4510.

The probes having the nucleotide sequence represented by SEQ ID NOS: 56063, 56064, 56065, 56066, 56067, 56068, 56069, 56070, 56071, 56072, 56073 were selected from the ORFs having the nucleotide sequence represented by SEQ ID NO: 4511.

The probes having the nucleotide sequence represented by SEQ ID NOS: 56074, 56075, 56076, 56077, 56078, 56079, 56080, 56081, 56082, 56083, 56084 were selected from the ORFs having the nucleotide sequence represented by SEQ ID NO: 4512.

The probes having the nucleotide sequence represented by SEQ ID NOS: 56085, 56086, 56087, 56088, 56089, 56090, 56091, 56092, 56093, 56094, 56095 were selected from the ORFs having the nucleotide sequence represented by SEQ ID NO: 4513.

The probes having the nucleotide sequence represented by SEQ ID NOS: 56096, 56097, 56098, 56099, 56100, 56101, 56102, 56103, 56104, 56105, 56106 were selected from the ORFs having the nucleotide sequence represented by SEQ ID NO: 4514.

The probes having the nucleotide sequence represented by SEQ ID NOS: 56107, 56108, 56109, 56110, 56111, 56112, 56113, 56114, 56115, 56116, 56117 were selected from the ORFs having the nucleotide sequence represented by SEQ ID NO: 4515.

The probes having the nucleotide sequence represented by SEQ ID NOS: 56118, 56119, 56120, 56121, 56122, 56123, 56124, 56125, 56126, 56127, 56128 were selected from the ORFs having the nucleotide sequence represented by SEQ ID NO: 4516.

The probes having the nucleotide sequence represented by SEQ ID NOS: 56129, 56130, 56131, 56132, 56133, 56134, 56135, 56136, 56137, 56138, 56139 were selected from the ORFs having the nucleotide sequence represented by SEQ ID NO: 4517.

The probes having the nucleotide sequence represented by SEQ ID NOS: 56140, 56141, 56142, 56143, 56144, 56145, 56146, 56147, 56148, 56149, 56150 were selected from the ORFs having the nucleotide sequence represented by SEQ ID NO: 4518.

The probes having the nucleotide sequence represented by SEQ ID NOS: 56151, 56152, 56153, 56154, 56155, 56156, 56157, 56158, 56159, 56160, 56161 were selected from the ORFs having the nucleotide sequence represented by SEQ ID NO: 4519.

The probes having the nucleotide sequence represented by SEQ ID NOS: 56162, 56163, 56164, 56165, 56166, 56167, 56168, 56169, 56170, 56171, 56172 were selected from the ORFs having the nucleotide sequence represented by SEQ ID NO: 4520.

The probes having the nucleotide sequence represented by SEQ ID NOS: 56173, 56174, 56175, 56176, 56177, 56178, 56179, 56180, 56181, 56182, 56183 were selected from the ORFs having the nucleotide sequence represented by SEQ ID NO: 4521.

The probes having the nucleotide sequence represented by SEQ ID NOS: 56184, 56185, 56186, 56187, 56188, 56189, 56190, 56191, 56192, 56193, 56194 were selected from the ORFs having the nucleotide sequence represented by SEQ ID NO: 4522.

The probes having the nucleotide sequence represented by SEQ ID NOS: 56195, 56196, 56197, 56198, 56199, 56200, 56201, 56202, 56203, 56204, 56205 were selected from the ORFs having the nucleotide sequence represented by SEQ ID NO: 4523.

The probes having the nucleotide sequence represented by SEQ ID NOS: 56206, 56207, 56208, 56209, 56210, 56211, 56212, 56213, 56214, 56215, 56216 were selected from the ORFs having the nucleotide sequence represented by SEQ ID NO: 4524.

The probes having the nucleotide sequence represented by SEQ ID NOS: 56217, 56218, 56219, 56220, 56221, 56222, 56223, 56224, 56225, 56226, 56227 were selected from the ORFs having the nucleotide sequence represented by SEQ ID NO: 4525.

The probes having the nucleotide sequence represented by SEQ ID NOS: 56228, 56229, 56230, 56231, 56232, 56233, 56234, 56235, 56236, 56237, 56238 were selected from the ORFs having the nucleotide sequence represented by SEQ ID NO: 4526.

The probes having the nucleotide sequence represented by SEQ ID NOS: 56239, 56240, 56241, 56242, 56243, 56244, 56245, 56246, 56247, 56248, 56249 were selected from the ORFs having the nucleotide sequence represented by SEQ ID NO: 4527.

The probes having the nucleotide sequence represented by SEQ ID NOS: 56250, 56251, 56252, 56253, 56254, 56255, 56256, 56257, 56258, 56259, 56260 were selected from the ORFs having the nucleotide sequence represented by SEQ ID NO: 4528.

The probes having the nucleotide sequence represented by SEQ ID NOS: 56261, 56262, 56263, 56264, 56265, 56266, 56267, 56268, 56269, 56270, 56271 were selected from the ORFs having the nucleotide sequence represented by SEQ ID NO: 4529.

The probes having the nucleotide sequence represented by SEQ ID NOS: 56272, 56273, 56274, 56275, 56276, 56277, 56278, 56279, 56280, 56281, 56282 were selected from the ORFs having the nucleotide sequence represented by SEQ ID NO: 4530.

The probes having the nucleotide sequence represented by SEQ ID NOS: 56283, 56284, 56285, 56286, 56287, 56288, 56289, 56290, 56291, 56292, 56293, 56294, 56295, 56296, 56297, 56298, 56299, 56300, 56301, 56302, 56303, 56304 were selected from the ORFs having the nucleotide sequence represented by SEQ ID NO: 4531.

The probes having the nucleotide sequence represented by SEQ ID NOS: 56305, 56306, 56307, 56308, 56309, 56310, 56311, 56312, 56313, 56314, 56315 were selected from the ORFs having the nucleotide sequence represented by SEQ ID NO: 4532.

The probes having the nucleotide sequence represented by SEQ ID NOS: 56316, 56317, 56318, 56319, 56320, 56321, 56322, 56323, 56324, 56325, 56326 were selected from the ORFs having the nucleotide sequence represented by SEQ ID NO: 4533.

The probes having the nucleotide sequence represented by SEQ ID NOS: 56327, 56328, 56329, 56330, 56331, 56332, 56333, 56334, 56335, 56336, 56337 were selected from the ORFs having the nucleotide sequence represented by SEQ ID NO: 4534.

The probes having the nucleotide sequence represented by SEQ ID NOS: 56338, 56339, 56340, 56341, 56342, 56343, 56344, 56345, 56346, 56347, 56348 were selected from the ORFs having the nucleotide sequence represented by SEQ ID NO: 4535.

The probes having the nucleotide sequence represented by SEQ ID NOS: 56349, 56350, 56351, 56352, 56353, 56354, 56355, 56356, 56357, 56358, 56359 were selected from the ORFs having the nucleotide sequence represented by SEQ ID NO: 4536.

The probes having the nucleotide sequence represented by SEQ ID NOS: 56360, 56361, 56362, 56363, 56364, 56365, 56366, 56367, 56368, 56369, 56370 were selected from the ORFs having the nucleotide sequence represented by SEQ ID NO: 4537.

The probes having the nucleotide sequence represented by SEQ ID NOS: 56371, 56372, 56373, 56374, 56375, 56376, 56377, 56378, 56379, 56380, 56381 were selected from the ORFs having the nucleotide sequence represented by SEQ ID NO: 4538.

The probes having the nucleotide sequence represented by SEQ ID NOS: 56382, 56383, 56384, 56385, 56386, 56387, 56388, 56389, 56390, 56391, 56392 were selected from the ORFs having the nucleotide sequence represented by SEQ ID NO: 4539.

The probes having the nucleotide sequence represented by SEQ ID NOS: 56393, 56394, 56395, 56396, 56397, 56398, 56399, 56400, 56401, 56402, 56403 were selected from the ORFs having the nucleotide sequence represented by SEQ ID NO: 4540.

The probes having the nucleotide sequence represented by SEQ ID NOS: 56404, 56405, 56406, 56407, 56408, 56409, 56410, 56411, 56412, 56413, 56414 were selected from the ORFs having the nucleotide sequence represented by SEQ ID NO: 4541.

The probes having the nucleotide sequence represented by SEQ ID NOS: 56415, 56416, 56417, 56418, 56419, 56420, 56421, 56422, 56423, 56424, 56425 were selected from the ORFs having the nucleotide sequence represented by SEQ ID NO: 4542.

The probes having the nucleotide sequence represented by SEQ ID NOS: 56426, 56427, 56428, 56429, 56430, 56431, 56432, 56433, 56434, 56435, 56436 were selected from the ORFs having the nucleotide sequence represented by SEQ ID NO: 4543.

The probes having the nucleotide sequence represented by SEQ ID NOS: 56437, 56438, 56439, 56440, 56441, 56442, 56443, 56444, 56445, 56446, 56447 were selected from the ORFs having the nucleotide sequence represented by SEQ ID NO: 4544.

The probes having the nucleotide sequence represented by SEQ ID NOS: 56448, 56449, 56450, 56451, 56452, 56453, 56454, 56455, 56456, 56457, 56458 were selected from the ORFs having the nucleotide sequence represented by SEQ ID NO: 4545.

The probes having the nucleotide sequence represented by SEQ ID NOS: 56459, 56460, 56461, 56462, 56463, 56464, 56465, 56466, 56467, 56468, 56469 were selected from the ORFs having the nucleotide sequence represented by SEQ ID NO: 4546.

The probes having the nucleotide sequence represented by SEQ ID NOS: 56470, 56471, 56472, 56473, 56474, 56475, 56476, 56477, 56478, 56479, 56480 were selected from the ORFs having the nucleotide sequence represented by SEQ ID NO: 4547.

The probes having the nucleotide sequence represented by SEQ ID NOS: 56481, 56482, 56483, 56484, 56485, 56486, 56487, 56488, 56489, 56490, 56491 were selected from the ORFs having the nucleotide sequence represented by SEQ ID NO: 4548.

The probes having the nucleotide sequence represented by SEQ ID NOS: 56492, 56493, 56494, 56495, 56496, 56497, 56498, 56499, 56500, 56501, 56502 were selected from the ORFs having the nucleotide sequence represented by SEQ ID NO: 4549.

The probes having the nucleotide sequence represented by SEQ ID NOS: 56503, 56504, 56505, 56506, 56507, 56508, 56509, 56510, 56511, 56512, 56513 were selected from the ORFs having the nucleotide sequence represented by SEQ ID NO: 4550.

The probes having the nucleotide sequence represented by SEQ ID NOS: 56514, 56515, 56516, 56517, 56518, 56519, 56520, 56521, 56522, 56523, 56524 were selected from the ORFs having the nucleotide sequence represented by SEQ ID NO: 4551.

The probes having the nucleotide sequence represented by SEQ ID NOS: 56525, 56526, 56527, 56528, 56529, 56530, 56531, 56532, 56533, 56534, 56535, 56536, 56537, 56538, 56539, 56540, 56541, 56542, 56543, 56544, 56545, 56546 were selected from the ORFs having the nucleotide sequence represented by SEQ ID NO: 4552.

The probes having the nucleotide sequence represented by SEQ ID NOS: 56547, 56548, 56549, 56550, 56551, 56552, 56553, 56554, 56555, 56556, 56557 were selected from the ORFs having the nucleotide sequence represented by SEQ ID NO: 4553.

The probes having the nucleotide sequence represented by SEQ ID NOS: 56558, 56559, 56560, 56561, 56562, 56563, 56564, 56565, 56566, 56567, 56568 were selected from the ORFs having the nucleotide sequence represented by SEQ ID NO: 4554.

The probes having the nucleotide sequence represented by SEQ ID NOS: 56569, 56570, 56571, 56572, 56573, 56574, 56575, 56576, 56577, 56578, 56579 were selected from the ORFs having the nucleotide sequence represented by SEQ ID NO: 4555.

The probes having the nucleotide sequence represented by SEQ ID NOS: 56580, 56581, 56582, 56583, 56584, 56585, 56586, 56587, 56588, 56589, 56590 were selected from the ORFs having the nucleotide sequence represented by SEQ ID NO: 4556.

The probes having the nucleotide sequence represented by SEQ ID NOS: 56591, 56592, 56593, 56594, 56595, 56596, 56597, 56598, 56599, 56600, 56601 were selected from the ORFs having the nucleotide sequence represented by SEQ ID NO: 4557.

The probes having the nucleotide sequence represented by SEQ ID NOS: 56602, 56603, 56604, 56605, 56606, 56607, 56608, 56609, 56610, 56611, 56612 were selected from the ORFs having the nucleotide sequence represented by SEQ ID NO: 4558.

The probes having the nucleotide sequence represented by SEQ ID NOS: 56613, 56614, 56615, 56616, 56617, 56618, 56619, 56620, 56621, 56622, 56623 were selected from the ORFs having the nucleotide sequence represented by SEQ ID NO: 4559.

The probes having the nucleotide sequence represented by SEQ ID NOS: 56624, 56625, 56626, 56627, 56628, 56629, 56630, 56631, 56632, 56633, 56634 were selected from the ORFs having the nucleotide sequence represented by SEQ ID NO: 4560.

The probes having the nucleotide sequence represented by SEQ ID NOS: 56635, 56636, 56637, 56638, 56639, 56640, 56641, 56642, 56643, 56644, 56645 were selected from the ORFs having the nucleotide sequence represented by SEQ ID NO: 4561.

The probes having the nucleotide sequence represented by SEQ ID NOS: 56646, 56647, 56648, 56649, 56650, 56651, 56652, 56653, 56654, 56655, 56656 were selected from the ORFs having the nucleotide sequence represented by SEQ ID NO: 4562.

The probes having the nucleotide sequence represented by SEQ ID NOS: 56657, 56658, 56659, 56660, 56661, 56662, 56663, 56664, 56665, 56666, 56667 were selected from the ORFs having the nucleotide sequence represented by SEQ ID NO: 4563.

The probes having the nucleotide sequence represented by SEQ ID NOS: 56668, 56669, 56670, 56671, 56672, 56673, 56674, 56675, 56676, 56677, 56678 were selected from the ORFs having the nucleotide sequence represented by SEQ ID NO: 4564.

The probes having the nucleotide sequence represented by SEQ ID NOS: 56679, 56680, 56681, 56682, 56683, 56684, 56685, 56686, 56687, 56688, 56689 were selected from the ORFs having the nucleotide sequence represented by SEQ ID NO: 4565.

The probes having the nucleotide sequence represented by SEQ ID NOS: 56690, 56691, 56692, 56693, 56694, 56695, 56696, 56697, 56698, 56699, 56700 were selected from the ORFs having the nucleotide sequence represented by SEQ ID NO: 4566.

The probes having the nucleotide sequence represented by SEQ ID NOS: 56701, 56702, 56703, 56704, 56705, 56706, 56707, 56708, 56709, 56710, 56711 were selected from the ORFs having the nucleotide sequence represented by SEQ ID NO: 4567.

The probes having the nucleotide sequence represented by SEQ ID NOS: 56712, 56713, 56714, 56715, 56716, 56717, 56718, 56719, 56720, 56721, 56722, 56723, 56724, 56725, 56726, 56727, 56728, 56729, 56730, 56731, 56732, 56733 were selected from the ORFs having the nucleotide sequence represented by SEQ ID NO: 4568.

The probes having the nucleotide sequence represented by SEQ ID NOS: 56734, 56735, 56736, 56737, 56738, 56739, 56740, 56741, 56742, 56743, 56744 were selected from the ORFs having the nucleotide sequence represented by SEQ ID NO: 4569.

The probes having the nucleotide sequence represented by SEQ ID NOS: 56745, 56746, 56747, 56748, 56749, 56750, 56751, 56752, 56753, 56754, 56755 were selected from the ORFs having the nucleotide sequence represented by SEQ ID NO: 4570.

The probes having the nucleotide sequence represented by SEQ ID NOS: 56756, 56757, 56758, 56759, 56760, 56761, 56762, 56763, 56764, 56765, 56766 were selected from the ORFs having the nucleotide sequence represented by SEQ ID NO: 4571.

The probes having the nucleotide sequence represented by SEQ ID NOS: 56767, 56768, 56769, 56770, 56771, 56772, 56773, 56774, 56775, 56776, 56777 were selected from the ORFs having the nucleotide sequence represented by SEQ ID NO: 4572.

The probes having the nucleotide sequence represented by SEQ ID NOS: 56778, 56779, 56780, 56781, 56782, 56783, 56784, 56785, 56786, 56787, 56788 were selected from the ORFs having the nucleotide sequence represented by SEQ ID NO: 4573.

The probes having the nucleotide sequence represented by SEQ ID NOS: 56789, 56790, 56791, 56792, 56793, 56794, 56795, 56796, 56797, 56798, 56799 were selected from the ORFs having the nucleotide sequence represented by SEQ ID NO: 4574.

The probes having the nucleotide sequence represented by SEQ ID NOS: 56800, 56801, 56802, 56803, 56804, 56805, 56806, 56807, 56808, 56809, 56810 were selected from the ORFs having the nucleotide sequence represented by SEQ ID NO: 4575.

The probes having the nucleotide sequence represented by SEQ ID NOS: 56811, 56812, 56813, 56814, 56815, 56816, 56817, 56818, 56819, 56820, 56821 were selected from the ORFs having the nucleotide sequence represented by SEQ ID NO: 4576.

The probes having the nucleotide sequence represented by SEQ ID NOS: 56822, 56823, 56824, 56825, 56826, 56827, 56828, 56829, 56830, 56831, 56832 were selected from the ORFs having the nucleotide sequence represented by SEQ ID NO: 4577.

The probes having the nucleotide sequence represented by SEQ ID NOS: 56833, 56834, 56835, 56836, 56837, 56838, 56839, 56840, 56841, 56842, 56843 were selected from the ORFs having the nucleotide sequence represented by SEQ ID NO: 4578.

The probes having the nucleotide sequence represented by SEQ ID NOS: 56844, 56845, 56846, 56847, 56848, 56849, 56850, 56851, 56852, 56853, 56854 were selected from the ORFs having the nucleotide sequence represented by SEQ ID NO: 4579.

The probes having the nucleotide sequence represented by SEQ ID NOS: 56855, 56856, 56857, 56858, 56859, 56860, 56861, 56862, 56863, 56864, 56865 were selected from the ORFs having the nucleotide sequence represented by SEQ ID NO: 4580.

The probes having the nucleotide sequence represented by SEQ ID NOS: 56866, 56867, 56868, 56869, 56870, 56871, 56872, 56873, 56874, 56875, 56876 were selected from the ORFs having the nucleotide sequence represented by SEQ ID NO: 4581.

The probes having the nucleotide sequence represented by SEQ ID NOS: 56877, 56878, 56879, 56880, 56881, 56882, 56883, 56884, 56885, 56886, 56887 were selected from the ORFs having the nucleotide sequence represented by SEQ ID NO: 4582.

The probes having the nucleotide sequence represented by SEQ ID NOS: 56888, 56889, 56890, 56891, 56892, 56893, 56894, 56895, 56896, 56897, 56898,were selected from the ORFs having the nucleotide sequence represented by SEQ ID NO: 4583.

The probes having the nucleotide sequence represented by SEQ ID NOS: 56899, 56900, 56901, 56902, 56903, 56904, 56905, 56906, 56907, 56908, 56909 were selected from the ORFs having the nucleotide sequence represented by SEQ ID NO: 4584.

The probes having the nucleotide sequence represented by SEQ ID NOS: 56910, 56911, 56912, 56913, 56914, 56915, 56916, 56917, 56918, 56919, 56920 were selected from the ORFs having the nucleotide sequence represented by SEQ ID NO: 4585.

The probes having the nucleotide sequence represented by SEQ ID NOS: 56921, 56922, 56923, 56924, 56925, 56926, 56927, 56928, 56929, 56930, 56931 were selected from the ORFs having the nucleotide sequence represented by SEQ ID NO: 4586.

The probes having the nucleotide sequence represented by SEQ ID NOS: 56932, 56933, 56934, 56935, 56936, 56937, 56938, 56939, 56940, 56941, 56942 were selected from the ORFs having the nucleotide sequence represented by SEQ ID NO: 4587.

The probes having the nucleotide sequence represented by SEQ ID NOS: 56943, 56944, 56945, 56946, 56947, 56948, 56949, 56950, 56951, 56952, 56953 were selected from the ORFs having the nucleotide sequence represented by SEQ ID NO: 4588.

The probes having the nucleotide sequence represented by SEQ ID NOS: 56954, 56955, 56956, 56957, 56958, 56959, 56960, 56961, 56962, 56963, 56964 were selected from the ORFs having the nucleotide sequence represented by SEQ ID NO: 4589.

The probes having the nucleotide sequence represented by SEQ ID NOS: 56965, 56966, 56967, 56968, 56969, 56970, 56971, 56972, 56973, 56974, 56975 were selected from the ORFs having the nucleotide sequence represented by SEQ ID NO: 4590.

The probes having the nucleotide sequence represented by SEQ ID NOS: 56976, 56977, 56978, 56979, 56980, 56981, 56982, 56983, 56984, 56985, 56986 were selected from the ORFs having the nucleotide sequence represented by SEQ ID NO: 4591.

The probes having the nucleotide sequence represented by SEQ ID NOS: 56987, 56988, 56989, 56990, 56991, 56992, 56993, 56994, 56995, 56996, 56997 were selected from the ORFs having the nucleotide sequence represented by SEQ ID NO: 4592.

The probes having the nucleotide sequence represented by SEQ ID NOS: 56998, 56999, 57000, 57001, 57002, 57003, 57004, 57005, 57006, 57007, 57008 were selected from the ORFs having the nucleotide sequence represented by SEQ ID NO: 4593.

The probes having the nucleotide sequence represented by SEQ ID NOS: 57009, 57010, 57011, 57012, 57013, 57014, 57015, 57016, 57017, 57018, 57019 were selected from the ORFs having the nucleotide sequence represented by SEQ ID NO: 4594.

The probes having the nucleotide sequence represented by SEQ ID NOS: 57020, 57021, 57022, 57023, 57024, 57025, 57026, 57027, 57028, 57029, 57030 were selected from the ORFs having the nucleotide sequence represented by SEQ ID NO: 4595.

The probes having the nucleotide sequence represented by SEQ ID NOS: 57031, 57032, 57033, 57034, 57035, 57036, 57037, 57038, 57039, 57040, 57041 were selected from the ORFs having the nucleotide sequence represented by SEQ ID NO: 4596.

The probes having the nucleotide sequence represented by SEQ ID NOS: 57042, 57043, 57044, 57045, 57046, 57047, 57048, 57049, 57050, 57051, 57052 were selected from the ORFs having the nucleotide sequence represented by SEQ ID NO: 4597.

The probes having the nucleotide sequence represented by SEQ ID NOS: 57053, 57054, 57055, 57056, 57057, 57058, 57059, 57060, 57061, 57062, 57063 were selected from the ORFs having the nucleotide sequence represented by SEQ ID NO: 4598.

The probes having the nucleotide sequence represented by SEQ ID NOS: 57064, 57065, 57066, 57067, 57068, 57069, 57070, 57071, 57072, 57073, 57074 were selected from the ORFs having the nucleotide sequence represented by SEQ ID NO: 4599.

The probes having the nucleotide sequence represented by SEQ ID NOS: 57075, 57076, 57077, 57078, 57079, 57080, 57081, 57082, 57083, 57084, 57085 were selected from the ORFs having the nucleotide sequence represented by SEQ ID NO: 4600.

The probes having the nucleotide sequence represented by SEQ ID NOS: 57086, 57087, 57088, 57089, 57090, 57091, 57092, 57093, 57094, 57095, 57096, 57097, 57098, 57099, 57100, 57101, 57102, 57103, 57104, 57105, 57106, 57107 were selected from the ORFs having the nucleotide sequence represented by SEQ ID NO: 4601.

The probes having the nucleotide sequence represented by SEQ ID NOS: 57108, 57109, 57110, 57111, 57112, 57113, 57114, 57115, 57116, 57117, 57118 were selected from the ORFs having the nucleotide sequence represented by SEQ ID NO: 4602.

The probes having the nucleotide sequence represented by SEQ ID NOS: 57119, 57120, 57121, 57122, 57123, 57124, 57125, 57126, 57127, 57128, 57129 were selected from the ORFs having the nucleotide sequence represented by SEQ ID NO: 4603.

The probes having the nucleotide sequence represented by SEQ ID NOS: 57130, 57131, 57132, 57133, 57134, 57135, 57136, 57137, 57138, 57139, 57140 were selected from the ORFs having the nucleotide sequence represented by SEQ ID NO: 4604.

The probes having the nucleotide sequence represented by SEQ ID NOS: 57141, 57142, 57143, 57144, 57145, 57146, 57147, 57148, 57149, 57150, 57151 were selected from the ORFs having the nucleotide sequence represented by SEQ ID NO: 4605.

The probes having the nucleotide sequence represented by SEQ ID NOS: 57152, 57153, 57154, 57155, 57156, 57157, 57158, 57159, 57160, 57161, 57162 were selected from the ORFs having the nucleotide sequence represented by SEQ ID NO: 4606.

The probes having the nucleotide sequence represented by SEQ ID NOS: 57163, 57164, 57165, 57166, 57167, 57168, 57169, 57170, 57171, 57172, 57173 were selected from the ORFs having the nucleotide sequence represented by SEQ ID NO: 4607.

The probes having the nucleotide sequence represented by SEQ ID NOS: 57174, 57175, 57176, 57177, 57178, 57179, 57180, 57181, 57182, 57183, 57184 were selected from the ORFs having the nucleotide sequence represented by SEQ ID NO: 4608.

The probes having the nucleotide sequence represented by SEQ ID NOS: 57185, 57186, 57187, 57188, 57189, 57190, 57191, 57192, 57193, 57194, 57195 were selected from the ORFs having the nucleotide sequence represented by SEQ ID NO: 4609.

The probes having the nucleotide sequence represented by SEQ ID NOS: 57196, 57197, 57198, 57199, 57200, 57201, 57202, 57203, 57204, 57205, 57206 were selected from the ORFs having the nucleotide sequence represented by SEQ ID NO: 4610.

The probes having the nucleotide sequence represented by SEQ ID NOS: 57207, 57208, 57209, 57210, 57211, 57212, 57213, 57214, 57215, 57216, 57217 were selected from the ORFs having the nucleotide sequence represented by SEQ ID NO: 4611.

The probes having the nucleotide sequence represented by SEQ ID NOS: 57218, 57219, 57220, 57221, 57222, 57223, 57224, 57225, 57226, 57227, 57228 were selected from the ORFs having the nucleotide sequence represented by SEQ ID NO: 4612.

The probes having the nucleotide sequence represented by SEQ ID NOS: 57229, 57230, 57231, 57232, 57233, 57234, 57235, 57236, 57237, 57238, 57239 were selected from the ORFs having the nucleotide sequence represented by SEQ ID NO: 4613.

The probes having the nucleotide sequence represented by SEQ ID NOS: 57240, 57241, 57242, 57243, 57244, 57245, 57246, 57247, 57248, 57249, 57250 were selected from the ORFs having the nucleotide sequence represented by SEQ ID NO: 4614.

The probes having the nucleotide sequence represented by SEQ ID NOS: 57251, 57252, 57253, 57254, 57255, 57256, 57257, 57258, 57259, 57260, 57261 were selected from the ORFs having the nucleotide sequence represented by SEQ ID NO: 4615.

The probes having the nucleotide sequence represented by SEQ ID NOS: 57262, 57263, 57264, 57265, 57266, 57267, 57268, 57269, 57270, 57271, 57272 were selected from the ORFs having the nucleotide sequence represented by SEQ ID NO: 4616.

The probes having the nucleotide sequence represented by SEQ ID NOS: 57273, 57274, 57275, 57276, 57277, 57278, 57279, 57280, 57281, 57282, 57283 were selected from the ORFs having the nucleotide sequence represented by SEQ ID NO: 4617.

The probes having the nucleotide sequence represented by SEQ ID NOS: 57284, 57285, 57286, 57287, 57288, 57289, 57290, 57291, 57292, 57293, 57294 were selected from the ORFs having the nucleotide sequence represented by SEQ ID NO: 4618.

The probes having the nucleotide sequence represented by SEQ ID NOS: 57295, 57296, 57297, 57298, 57299, 57300, 57301, 57302, 57303, 57304, 57305 were selected from the ORFs having the nucleotide sequence represented by SEQ ID NO: 4619.

The probes having the nucleotide sequence represented by SEQ ID NOS: 57306, 57307, 57308, 57309, 57310, 57311, 57312, 57313, 57314, 57315, 57316 were selected from the ORFs having the nucleotide sequence represented by SEQ ID NO: 4620.

The probes having the nucleotide sequence represented by SEQ ID NOS: 57317, 57318, 57319, 57320, 57321, 57322, 57323, 57324, 57325, 57326, 57327 were selected from the ORFs having the nucleotide sequence represented by SEQ ID NO: 4621.

The probes having the nucleotide sequence represented by SEQ ID NOS: 57328, 57329, 57330, 57331, 57332, 57333, 57334, 57335, 57336, 57337, 57338 were selected from the ORFs having the nucleotide sequence represented by SEQ ID NO: 4622.

The probes having the nucleotide sequence represented by SEQ ID NOS: 57339, 57340, 57341, 57342, 57343, 57344, 57345, 57346, 57347, 57348, 57349 were selected from the ORFs having the nucleotide sequence represented by SEQ ID NO: 4623.

The probes having the nucleotide sequence represented by SEQ ID NOS: 57350, 57351, 57352, 57353, 57354, 57355, 57356, 57357, 57358, 57359, 57360 were selected from the ORFs having the nucleotide sequence represented by SEQ ID NO: 4624.

The probes having the nucleotide sequence represented by SEQ ID NOS: 57361, 57362, 57363, 57364, 57365, 57366, 57367, 57368, 57369, 57370, 57371 were selected from the ORFs having the nucleotide sequence represented by SEQ ID NO: 4625.

The probes having the nucleotide sequence represented by SEQ ID NOS: 57372, 57373, 57374, 57375, 57376, 57377, 57378, 57379, 57380, 57381, 57382 were selected from the ORFs having the nucleotide sequence represented by SEQ ID NO: 4626.

The probes having the nucleotide sequence represented by SEQ ID NOS: 57383, 57384, 57385, 57386, 57387, 57388, 57389, 57390, 57391, 57392, 57393 were selected from the ORFs having the nucleotide sequence represented by SEQ ID NO: 4627.

The probes having the nucleotide sequence represented by SEQ ID NOS: 57394, 57395, 57396, 57397, 57398, 57399, 57400, 57401, 57402, 57403, 57404 were selected from the ORFs having the nucleotide sequence represented by SEQ ID NO: 4628.

The probes having the nucleotide sequence represented by SEQ ID NOS: 57405, 57406, 57407, 57408, 57409, 57410, 57411, 57412, 57413, 57414, 57415 were selected from the ORFs having the nucleotide sequence represented by SEQ ID NO: 4629.

The probes having the nucleotide sequence represented by SEQ ID NOS: 57416, 57417, 57418, 57419, 57420, 57421, 57422, 57423, 57424, 57425, 57426 were selected from the ORFs having the nucleotide sequence represented by SEQ ID NO: 4630.

The probes having the nucleotide sequence represented by SEQ ID NOS: 57427, 57428, 57429, 57430, 57431, 57432, 57433, 57434, 57435, 57436, 57437 were selected from the ORFs having the nucleotide sequence represented by SEQ ID NO: 4631.

The probes having the nucleotide sequence represented by SEQ ID NOS: 57438, 57439, 57440, 57441, 57442, 57443, 57444, 57445, 57446, 57447, 57448 were selected from the ORFs having the nucleotide sequence represented by SEQ ID NO: 4632.

The probes having the nucleotide sequence represented by SEQ ID NOS: 57449, 57450, 57451, 57452, 57453, 57454, 57455, 57456, 57457, 57458, 57459 were selected from the ORFs having the nucleotide sequence represented by SEQ ID NO: 4633.

The probes having the nucleotide sequence represented by SEQ ID NOS: 57460, 57461, 57462, 57463, 57464, 57465, 57466, 57467, 57468, 57469, 57470 were selected from the ORFs having the nucleotide sequence represented by SEQ ID NO: 4634.

The probes having the nucleotide sequence represented by SEQ ID NOS: 57471, 57472, 57473, 57474, 57475, 57476, 57477, 57478, 57479, 57480, 57481 were selected from the ORFs having the nucleotide sequence represented by SEQ ID NO: 4635.

The probes having the nucleotide sequence represented by SEQ ID NOS: 57482, 57483, 57484, 57485, 57486, 57487, 57488, 57489, 57490, 57491, 57492 were selected from the ORFs having the nucleotide sequence represented by SEQ ID NO: 4636.

The probes having the nucleotide sequence represented by SEQ ID NOS: 57493, 57494, 57495, 57496, 57497, 57498, 57499, 57500, 57501, 57502, 57503 were selected from the ORFs having the nucleotide sequence represented by SEQ ID NO: 4637.

The probes having the nucleotide sequence represented by SEQ ID NOS: 57504, 57505, 57506, 57507, 57508, 57509, 57510, 57511, 57512, 57513, 57514 were selected from the ORFs having the nucleotide sequence represented by SEQ ID NO: 4638.

The probes having the nucleotide sequence represented by SEQ ID NOS: 57515, 57516, 57517, 57518, 57519, 57520, 57521, 57522, 57523, 57524, 57525 were selected from the ORFs having the nucleotide sequence represented by SEQ ID NO: 4639.

The probes having the nucleotide sequence represented by SEQ ID NOS: 57526, 57527, 57528, 57529, 57530, 57531, 57532, 57533, 57534, 57535, 57536 were selected from the ORFs having the nucleotide sequence represented by SEQ ID NO: 4640.

The probes having the nucleotide sequence represented by SEQ ID NOS: 57537, 57538, 57539, 57540, 57541, 57542, 57543, 57544, 57545, 57546, 57547 were selected from the ORFs having the nucleotide sequence represented by SEQ ID NO: 4641.

The probes having the nucleotide sequence represented by SEQ ID NOS: 57548, 57549, 57550, 57551, 57552, 57553, 57554, 57555, 57556, 57557, 57558 were selected from the ORFs having the nucleotide sequence represented by SEQ ID NO: 4642.

The probes having the nucleotide sequence represented by SEQ ID NOS: 57559, 57560, 57561, 57562, 57563, 57564, 57565, 57566, 57567, 57568, 57569 were selected from the ORFs having the nucleotide sequence represented by SEQ ID NO: 4643.

The probes having the nucleotide sequence represented by SEQ ID NOS: 57570, 57571, 57572, 57573, 57574, 57575, 57576, 57577, 57578, 57579, 57580 were selected from the ORFs having the nucleotide sequence represented by SEQ ID NO: 4644.

The probes having the nucleotide sequence represented by SEQ ID NOS: 57581, 57582, 57583, 57584, 57585, 57586, 57587, 57588, 57589, 57590, 57591 were selected from the ORFs having the nucleotide sequence represented by SEQ ID NO: 4645.

The probes having the nucleotide sequence represented by SEQ ID NOS: 57592, 57593, 57594, 57595, 57596, 57597, 57598, 57599, 57600, 57601, 57602 were selected from the ORFs having the nucleotide sequence represented by SEQ ID NO: 4646.

The probes having the nucleotide sequence represented by SEQ ID NOS: 57603, 57604, 57605, 57606, 57607, 57608, 57609, 57610, 57611, 57612, 57613 were selected from the ORFs having the nucleotide sequence represented by SEQ ID NO: 4647.

The probes having the nucleotide sequence represented by SEQ ID NOS: 57614, 57615, 57616, 57617, 57618, 57619, 57620, 57621, 57622, 57623, 57624 were selected from the ORFs having the nucleotide sequence represented by SEQ ID NO: 4648.

The probes having the nucleotide sequence represented by SEQ ID NOS: 57625, 57626, 57627, 57628, 57629, 57630, 57631, 57632, 57633, 57634, 57635 were selected from the ORFs having the nucleotide sequence represented by SEQ ID NO: 4649.

The probes having the nucleotide sequence represented by SEQ ID NOS: 57636, 57637, 57638, 57639, 57640, 57641, 57642, 57643, 57644, 57645, 57646 were selected from the ORFs having the nucleotide sequence represented by SEQ ID NO: 4650.

The probes having the nucleotide sequence represented by SEQ ID NOS: 57647, 57648, 57649, 57650, 57651, 57652, 57653, 57654, 57655, 57656, 57657 were selected from the ORFs having the nucleotide sequence represented by SEQ ID NO: 4651.

The probes having the nucleotide sequence represented by SEQ ID NOS: 57658, 57659, 57660, 57661, 57662, 57663, 57664, 57665, 57666, 57667, 57668 were selected from the ORFs having the nucleotide sequence represented by SEQ ID NO: 4652.

The probes having the nucleotide sequence represented by SEQ ID NOS: 57669, 57670, 57671, 57672, 57673, 57674, 57675, 57676, 57677, 57678, 57679 were selected from the ORFs having the nucleotide sequence represented by SEQ ID NO: 4653.

The probes having the nucleotide sequence represented by SEQ ID NOS: 57680, 57681, 57682, 57683, 57684, 57685, 57686, 57687, 57688, 57689, 57690 were selected from the ORFs having the nucleotide sequence represented by SEQ ID NO: 4654.

The probes having the nucleotide sequence represented by SEQ ID NOS: 57691, 57692, 57693, 57694, 57695, 57696, 57697, 57698, 57699, 57700, 57701 were selected from the ORFs having the nucleotide sequence represented by SEQ ID NO: 4655.

The probes having the nucleotide sequence represented by SEQ ID NOS: 57702, 57703, 57704, 57705, 57706, 57707, 57708, 57709, 57710, 57711, 57712 were selected from the ORFs having the nucleotide sequence represented by SEQ ID NO: 4656.

The probes having the nucleotide sequence represented by SEQ ID NOS: 57713, 57714, 57715, 57716, 57717, 57718, 57719, 57720, 57721, 57722, 57723 were selected from the ORFs having the nucleotide sequence represented by SEQ ID NO: 4657.

The probes having the nucleotide sequence represented by SEQ ID NOS: 57724, 57725, 57726, 57727, 57728, 57729, 57730, 57731, 57732, 57733, 57734 were selected from the ORFs having the nucleotide sequence represented by SEQ ID NO: 4658.

The probes having the nucleotide sequence represented by SEQ ID NOS: 57735, 57736, 57737, 57738, 57739, 57740, 57741, 57742, 57743, 57744, 57745 were selected from the ORFs having the nucleotide sequence represented by SEQ ID NO: 4659.

The probes having the nucleotide sequence represented by SEQ ID NOS: 57746, 57747, 57748, 57749, 57750, 57751, 57752, 57753, 57754, 57755, 57756 were selected from the ORFs having the nucleotide sequence represented by SEQ ID NO: 4660.

The probes having the nucleotide sequence represented by SEQ ID NOS: 57757, 57758, 57759, 57760, 57761, 57762, 57763, 57764, 57765, 57766, 57767 were selected from the ORFs having the nucleotide sequence represented by SEQ ID NO: 4661.

The probes having the nucleotide sequence represented by SEQ ID NOS: 57768, 57769, 57770, 57771, 57772, 57773, 57774, 57775, 57776, 57777, 57778 were selected from the ORFs having the nucleotide sequence represented by SEQ ID NO: 4662.

The probes having the nucleotide sequence represented by SEQ ID NOS: 57779, 57780, 57781, 57782, 57783, 57784, 57785, 57786, 57787, 57788, 57789 were selected from the ORFs having the nucleotide sequence represented by SEQ ID NO: 4663.

The probes having the nucleotide sequence represented by SEQ ID NOS: 57790, 57791, 57792, 57793, 57794, 57795, 57796, 57797, 57798, 57799, 57800 were selected from the ORFs having the nucleotide sequence represented by SEQ ID NO: 4664.

The probes having the nucleotide sequence represented by SEQ ID NOS: 57801, 57802, 57803, 57804, 57805, 57806, 57807, 57808, 57809, 57810, 57811 were selected from the ORFs having the nucleotide sequence represented by SEQ ID NO: 4665.

The probes having the nucleotide sequence represented by SEQ ID NOS: 57812, 57813, 57814, 57815, 57816, 57817, 57818, 57819, 57820, 57821, 57822 were selected from the ORFs having the nucleotide sequence represented by SEQ ID NO: 4666.

The probes having the nucleotide sequence represented by SEQ ID NOS: 57823, 57824, 57825, 57826, 57827, 57828, 57829, 57830, 57831, 57832, 57833 were selected from the ORFs having the nucleotide sequence represented by SEQ ID NO: 4667.

The probes having the nucleotide sequence represented by SEQ ID NOS: 57834, 57835, 57836, 57837, 57838, 57839, 57840, 57841, 57842, 57843, 57844 were selected from the ORFs having the nucleotide sequence represented by SEQ ID NO: 4668.

The probes having the nucleotide sequence represented by SEQ ID NOS: 57845, 57846, 57847, 57848, 57849, 57850, 57851, 57852, 57853, 57854, 57855 were selected from the ORFs having the nucleotide sequence represented by SEQ ID NO: 4669.

The probes having the nucleotide sequence represented by SEQ ID NOS: 57856, 57857, 57858, 57859, 57860, 57861, 57862, 57863, 57864, 57865, 57866 were selected from the ORFs having the nucleotide sequence represented by SEQ ID NO: 4670.

The probes having the nucleotide sequence represented by SEQ ID NOS: 57867, 57868, 57869, 57870, 57871, 57872, 57873, 57874, 57875, 57876, 57877 were selected from the ORFs having the nucleotide sequence represented by SEQ ID NO: 4671.

The probes having the nucleotide sequence represented by SEQ ID NOS: 57878, 57879, 57880, 57881, 57882, 57883, 57884, 57885, 57886, 57887, 57888 were selected from the ORFs having the nucleotide sequence represented by SEQ ID NO: 4672.

The probes having the nucleotide sequence represented by SEQ ID NOS: 57889, 57890, 57891, 57892, 57893, 57894, 57895, 57896, 57897, 57898, 57899 were selected from the ORFs having the nucleotide sequence represented by SEQ ID NO: 4673.

The probes having the nucleotide sequence represented by SEQ ID NOS: 57900, 57901, 57902, 57903, 57904, 57905, 57906, 57907, 57908, 57909, 57910 were selected from the ORFs having the nucleotide sequence represented by SEQ ID NO: 4674.

The probes having the nucleotide sequence represented by SEQ ID NOS: 57911, 57912, 57913, 57914, 57915, 57916, 57917, 57918, 57919, 57920, 57921 were selected from the ORFs having the nucleotide sequence represented by SEQ ID NO: 4675.

The probes having the nucleotide sequence represented by SEQ ID NOS: 57922, 57923, 57924, 57925, 57926, 57927, 57928, 57929, 57930, 57931, 57932 were selected from the ORFs having the nucleotide sequence represented by SEQ ID NO: 4676.

The probes having the nucleotide sequence represented by SEQ ID NOS: 57933, 57934, 57935, 57936, 57937, 57938, 57939, 57940, 57941, 57942, 57943 were selected from the ORFs having the nucleotide sequence represented by SEQ ID NO: 4677.

The probes having the nucleotide sequence represented by SEQ ID NOS: 57944, 57945, 57946, 57947, 57948, 57949, 57950, 57951, 57952, 57953, 57954 were selected from the ORFs having the nucleotide sequence represented by SEQ ID NO: 4678.

The probes having the nucleotide sequence represented by SEQ ID NOS: 57955, 57956, 57957, 57958, 57959, 57960, 57961, 57962, 57963, 57964, 57965 were selected from the ORFs having the nucleotide sequence represented by SEQ ID NO: 4679.

The probes having the nucleotide sequence represented by SEQ ID NOS: 57966, 57967, 57968, 57969, 57970, 57971, 57972, 57973, 57974, 57975, 57976 were selected from the ORFs having the nucleotide sequence represented by SEQ ID NO: 4680.

The probes having the nucleotide sequence represented by SEQ ID NOS: 57977, 57978, 57979, 57980, 57981, 57982, 57983, 57984, 57985, 57986, 57987 were selected from the ORFs having the nucleotide sequence represented by SEQ ID NO: 4681.

The probes having the nucleotide sequence represented by SEQ ID NOS: 57988, 57989, 57990, 57991, 57992, 57993, 57994, 57995, 57996, 57997, 57998 were selected from the ORFs having the nucleotide sequence represented by SEQ ID NO: 4682.

The probes having the nucleotide sequence represented by SEQ ID NOS: 57999, 58000, 58001, 58002, 58003, 58004, 58005, 58006, 58007, 58008, 58009 were selected from the ORFs having the nucleotide sequence represented by SEQ ID NO: 4683.

The probes having the nucleotide sequence represented by SEQ ID NOS: 58010, 58011, 58012, 58013, 58014, 58015, 58016, 58017, 58018, 58019, 58020 were selected from the ORFs having the nucleotide sequence represented by SEQ ID NO: 4684.

The probes having the nucleotide sequence represented by SEQ ID NOS: 58021, 58022, 58023, 58024, 58025, 58026, 58027, 58028, 58029, 58030, 58031 were selected from the ORFs having the nucleotide sequence represented by SEQ ID NO: 4685.

The probes having the nucleotide sequence represented by SEQ ID NOS: 58032, 58033, 58034, 58035, 58036, 58037, 58038, 58039, 58040, 58041, 58042 were selected from the ORFs having the nucleotide sequence represented by SEQ ID NO: 4686.

The probes having the nucleotide sequence represented by SEQ ID NOS: 58043, 58044, 58045, 58046, 58047, 58048, 58049, 58050, 58051, 58052, 58053 were selected from the ORFs having the nucleotide sequence represented by SEQ ID NO: 4687.

The probes having the nucleotide sequence represented by SEQ ID NOS: 58054, 58055, 58056, 58057, 58058, 58059, 58060, 58061, 58062, 58063, 58064 were selected from the ORFs having the nucleotide sequence represented by SEQ ID NO: 4688.

The probes having the nucleotide sequence represented by SEQ ID NOS: 58065, 58066, 58067, 58068, 58069, 58070, 58071, 58072, 58073, 58074, 58075 were selected from the ORFs having the nucleotide sequence represented by SEQ ID NO: 4689.

The probes having the nucleotide sequence represented by SEQ ID NOS: 58076, 58077, 58078, 58079, 58080, 58081, 58082, 58083, 58084, 58085, 58086 were selected from the ORFs having the nucleotide sequence represented by SEQ ID NO: 4690.

The probes having the nucleotide sequence represented by SEQ ID NOS: 58087, 58088, 58089, 58090, 58091, 58092, 58093, 58094, 58095, 58096, 58097 were selected from the ORFs having the nucleotide sequence represented by SEQ ID NO: 4691.

The probes having the nucleotide sequence represented by SEQ ID NOS: 58098, 58099, 58100, 58101, 58102, 58103, 58104, 58105, 58106, 58107, 58108 were selected from the ORFs having the nucleotide sequence represented by SEQ ID NO: 4692.

The probes having the nucleotide sequence represented by SEQ ID NOS: 58109, 58110, 58111, 58112, 58113, 58114, 58115, 58116, 58117, 58118, 58119 were selected from the ORFs having the nucleotide sequence represented by SEQ ID NO: 4693.

The probes having the nucleotide sequence represented by SEQ ID NOS: 58120, 58121, 58122, 58123, 58124, 58125, 58126, 58127, 58128, 58129, 58130 were selected from the ORFs having the nucleotide sequence represented by SEQ ID NO: 4694.

The probes having the nucleotide sequence represented by SEQ ID NOS: 58131, 58132, 58133, 58134, 58135, 58136, 58137, 58138, 58139, 58140, 58141 were selected from the ORFs having the nucleotide sequence represented by SEQ ID NO: 4695.

The probes having the nucleotide sequence represented by SEQ ID NOS: 58142, 58143, 58144, 58145, 58146, 58147, 58148, 58149, 58150, 58151, 58152 were selected from the ORFs having the nucleotide sequence represented by SEQ ID NO: 4696.

The probes having the nucleotide sequence represented by SEQ ID NOS: 58153, 58154, 58155, 58156, 58157, 58158, 58159, 58160, 58161, 58162, 58163 were selected from the ORFs having the nucleotide sequence represented by SEQ ID NO: 4697.

The probes having the nucleotide sequence represented by SEQ ID NOS: 58164, 58165, 58166, 58167, 58168, 58169, 58170, 58171, 58172, 58173, 58174 were selected from the ORFs having the nucleotide sequence represented by SEQ ID NO: 4698.

The probes having the nucleotide sequence represented by SEQ ID NOS: 58175, 58176, 58177, 58178, 58179, 58180, 58181, 58182, 58183, 58184, 58185 were selected from the ORFs having the nucleotide sequence represented by SEQ ID NO: 4699.

The probes having the nucleotide sequence represented by SEQ ID NOS: 58186, 58187, 58188, 58189, 58190, 58191, 58192, 58193, 58194, 58195, 58196 were selected from the ORFs having the nucleotide sequence represented by SEQ ID NO: 4700.

The probes having the nucleotide sequence represented by SEQ ID NOS: 58197, 58198, 58199, 58200, 58201, 58202, 58203, 58204, 58205, 58206, 58207 were selected from the ORFs having the nucleotide sequence represented by SEQ ID NO: 4701.

The probes having the nucleotide sequence represented by SEQ ID NOS: 58208, 58209, 58210, 58211, 58212, 58213, 58214, 58215, 58216, 58217, 58218 were selected from the ORFs having the nucleotide sequence represented by SEQ ID NO: 4702.

The probes having the nucleotide sequence represented by SEQ ID NOS: 58219, 58220, 58221, 58222, 58223, 58224, 58225, 58226, 58227, 58228, 58229 were selected from the ORFs having the nucleotide sequence represented by SEQ ID NO: 4703.

The probes having the nucleotide sequence represented by SEQ ID NOS: 58230, 58231, 58232, 58233, 58234, 58235, 58236, 58237, 58238, 58239, 58240 were selected from the ORFs having the nucleotide sequence represented by SEQ ID NO: 4704.

The probes having the nucleotide sequence represented by SEQ ID NOS: 58241, 58242, 58243, 58244,.58245, 58246, 58247, 58248, 58249, 58250, 58251 were selected from the ORFs having the nucleotide sequence represented by SEQ ID NO: 4705.

The probes having the nucleotide sequence represented by SEQ ID NOS: 58252, 58253, 58254, 58255, 58256, 58257, 58258, 58259, 58260, 58261, 58262 were selected from the ORFs having the nucleotide sequence represented by SEQ ID NO: 4706.

The probes having the nucleotide sequence represented by SEQ ID NOS: 58263, 58264, 58265, 58266, 58267, 58268, 58269, 58270, 58271, 58272, 58273 were selected from the ORFs having the nucleotide sequence represented by SEQ ID NO: 4707.

The probes having the nucleotide sequence represented by SEQ ID NOS: 58274, 58275, 58276, 58277, 58278, 58279, 58280, 58281, 58282, 58283, 58284 were selected from the ORFs having the nucleotide sequence represented by SEQ ID NO: 4708.

The probes having the nucleotide sequence represented by SEQ ID NOS: 58285, 58286, 58287, 58288, 58289, 58290, 58291, 58292, 58293, 58294, 58295 were selected from the ORFs having the nucleotide sequence represented by SEQ ID NO: 4709.

The probes having the nucleotide sequence represented by SEQ ID NOS: 58296, 58297, 58298, 58299, 58300, 58301, 58302, 58303, 58304, 58305, 58306 were selected from the ORFs having the nucleotide sequence represented by SEQ ID NO: 4710.

The probes having the nucleotide sequence represented by SEQ ID NOS: 58307, 58308, 58309, 58310, 58311, 58312, 58313, 58314, 58315, 58316, 58317 were selected from the ORFs having the nucleotide sequence represented by SEQ ID NO: 4711.

The probes having the nucleotide sequence represented by SEQ ID NOS: 58318, 58319, 58320, 58321, 58322, 58323, 58324, 58325, 58326, 58327, 58328 were selected from the ORFs having the nucleotide sequence represented by SEQ ID NO: 4712.

The probes having the nucleotide sequence represented by SEQ ID NOS: 58329, 58330, 58331, 58332, 58333, 58334, 58335, 58336, 58337, 58338, 58339 were selected from the ORFs having the nucleotide sequence represented by SEQ ID NO: 4713.

The probes having the nucleotide sequence represented by SEQ ID NOS: 58340, 58341, 58342, 58343, 58344, 58345, 58346, 58347, 58348, 58349, 58350 were selected from the ORFs having the nucleotide sequence represented by SEQ ID NO: 4714.

The probes having the nucleotide sequence represented by SEQ ID NOS: 58351, 58352, 58353, 58354, 58355, 58356, 58357, 583.58, 58359, 58360, 58361 were selected from the ORFs having the nucleotide sequence represented by SEQ ID NO: 4715.

The probes having the nucleotide sequence represented by SEQ ID NOS: 58362, 58363, 58364, 58365, 58366, 58367, 58368, 58369, 58370, 58371, 58372 were selected from the ORFs having the nucleotide sequence represented by SEQ ID NO: 4716.

The probes having the nucleotide sequence represented by SEQ ID NOS: 58373, 58374, 58375, 58376, 58377, 58378, 58379, 58380, 58381, 58382, 58383 were selected from the ORFs having the nucleotide sequence represented by SEQ ID NO: 4717.

The probes having the nucleotide sequence represented by SEQ ID NOS: 58384, 58385, 58386, 58387, 58388, 58389, 58390, 58391, 58392, 58393, 58394 were selected from the ORFs having the nucleotide sequence represented by SEQ ID NO: 4718.

The probes having the nucleotide sequence represented by SEQ ID NOS: 58395, 58396, 58397, 58398, 58399, 58400, 58401, 58402, 58403, 58404, 58405 were selected from the ORFs having the nucleotide sequence represented by SEQ ID NO: 4719.

The probes having the nucleotide sequence represented by SEQ ID NOS: 58406, 58407, 58408, 58409, 58410, 58411, 58412, 58413, 58414, 58415, 58416 were selected from the ORFs having the nucleotide sequence represented by SEQ ID NO: 4720.

The probes having the nucleotide sequence represented by SEQ ID NOS: 58417, 58418, 58419, 58420, 58421, 58422, 58423, 58424, 58425, 58426, 58427 were selected from the ORFs having the nucleotide sequence represented by SEQ ID NO: 4721.

The probes having the nucleotide sequence represented by SEQ ID NOS: 58428, 58429, 58430, 58431, 58432, 58433, 58434, 58435, 58436, 58437, 58438 were selected from the ORFs having the nucleotide sequence represented by SEQ ID NO: 4722.

The probes having the nucleotide sequence represented by SEQ ID NOS: 58439, 58440, 58441, 58442, 58443, 58444, 58445, 58446, 58447, 58448, 58449 were selected from the ORFs having the nucleotide sequence represented by SEQ ID NO: 4723.

The probes having the nucleotide sequence represented by SEQ ID NOS: 58450, 58451, 58452, 58453, 58454, 58455, 58456, 58457, 58458, 58459, 58460 were selected from the ORFs having the nucleotide sequence represented by SEQ ID NO: 4724.

The probes having the nucleotide sequence represented by SEQ ID NOS: 58461, 58462, 58463, 58464, 58465, 58466, 58467, 58468, 58469, 58470, 58471 were selected from the ORFs having the nucleotide sequence represented by SEQ ID NO: 4725.

The probes having the nucleotide sequence represented by SEQ ID NOS: 58472, 58473, 58474, 58475, 58476, 58477, 58478, 58479, 58480, 58481, 58482 were selected from the ORFs having the nucleotide sequence represented by SEQ ID NO: 4726.

The probes having the nucleotide sequence represented by SEQ ID NOS: 58483, 58484, 58485, 58486, 58487, 58488, 58489, 58490, 58491, 58492, 58493 were selected from the ORFs having the nucleotide sequence represented by SEQ ID NO: 4727.

The probes having the nucleotide sequence represented by SEQ ID NOS: 58494, 58495, 58496, 58497, 58498, 58499, 58500, 58501, 58502, 58503, 58504 were selected from the ORFs having the nucleotide sequence represented by SEQ ID NO: 4728.

The probes having the nucleotide sequence represented by SEQ ID NOS: 58505, 58506, 58507, 58508, 58509, 58510, 58511, 58512, 58513, 58514, 58515 were selected from the ORFs having the nucleotide sequence represented by SEQ ID NO: 4729.

The probes having the nucleotide sequence represented by SEQ ID NOS: 58516, 58517, 58518, 58519, 58520, 58521, 58522, 58523, 58524, 58525, 58526 were selected from the ORFs having the nucleotide sequence represented by SEQ ID NO: 4730.

The probes having the nucleotide sequence represented by SEQ ID NOS: 58527, 58528, 58529, 58530, 58531, 58532, 58533, 58534, 58535, 58536, 58537 were selected from the ORFs having the nucleotide sequence represented by SEQ ID NO: 4731.

The probes having the nucleotide sequence represented by SEQ ID NOS: 58538, 58539, 58540, 58541, 58542, 58543, 58544, 58545, 58546, 58547, 58548 were selected from the ORFs having the nucleotide sequence represented by SEQ ID NO: 4732.

The probes having the nucleotide sequence represented by SEQ ID NOS: 58549, 58550, 58551, 58552, 58553, 58554, 58555, 58556, 58557, 58558, 58559 were selected from the ORFs having the nucleotide sequence represented by SEQ ID NO: 4733.

The probes having the nucleotide sequence represented by SEQ ID NOS: 58560, 58561, 58562, 58563, 58564, 58565, 58566, 58567, 58568, 58569, 58570 were selected from the ORFs having the nucleotide sequence represented by SEQ ID NO: 4734.

The probes having the nucleotide sequence represented by SEQ ID NOS: 58571, 58572, 58573, 58574, 58575, 58576, 58577, 58578, 58579, 58580, 58581 were selected from the ORFs having the nucleotide sequence represented by SEQ ID NO: 4735.

The probes having the nucleotide sequence represented by SEQ ID NOS: 58582, 58583, 58584, 58585, 58586, 58587, 58588, 58589, 58590, 58591, 58592 were selected from the ORFs having the nucleotide sequence represented by SEQ ID NO: 4736.

The probes having the nucleotide sequence represented by SEQ ID NOS: 58593, 58594, 58595, 58596, 58597, 58598, 58599, 58600, 58601, 58602, 58603 were selected from the ORFs having the nucleotide sequence represented by SEQ ID NO: 4737.

The probes having the nucleotide sequence represented by SEQ ID NOS: 58604, 58605, 58606, 58607, 58608, 58609, 58610, 58611, 58612, 58613, 58614 were selected from the ORFs having the nucleotide sequence represented by SEQ ID NO: 4738.

The probes having the nucleotide sequence represented by SEQ ID NOS: 58615, 58616, 58617, 58618, 58619, 58620, 58621, 58622, 58623, 58624, 58625 were selected from the ORFs having the nucleotide sequence represented by SEQ ID NO: 4739.

The probes having the nucleotide sequence represented by SEQ ID NOS: 58626, 58627, 58628, 58629, 58630, 58631, 58632, 58633, 58634, 58635, 58636 were selected from the ORFs having the nucleotide sequence represented by SEQ ID NO: 4740.

The probes having the nucleotide sequence represented by SEQ ID NOS: 58637, 58638, 58639, 58640, 58641, 58642, 58643, 58644, 58645, 58646, 58647 were selected from the ORFs having the nucleotide sequence represented by SEQ ID NO: 4741.

The probes having the nucleotide sequence represented by SEQ ID NOS: 58648, 58649, 58650, 58651, 58652, 58653, 58654, 58655, 58656, 58657, 58658 were selected from the ORFs having the nucleotide sequence represented by SEQ ID NO: 4742.

The probes having the nucleotide sequence represented by SEQ ID NOS: 58659, 58660, 58661, 58662, 58663, 58664, 58665, 58666, 58667, 58668, 58669 were selected from the ORFs having the nucleotide sequence represented by SEQ ID NO: 4743.

The probes having the nucleotide sequence represented by SEQ ID NOS: 58670, 58671, 58672, 58673, 58674, 58675, 58676, 58677, 58678, 58679, 58680 were selected from the ORFs having the nucleotide sequence represented by SEQ ID NO: 4744.

The probes having the nucleotide sequence represented by SEQ ID NOS: 58681, 58682, 58683, 58684, 58685, 58686, 58687, 58688, 58689, 58690, 58691 were selected from the ORFs having the nucleotide sequence represented by SEQ ID NO: 4745.

The probes having the nucleotide sequence represented by SEQ ID NOS: 58692, 58693, 58694, 58695, 58696, 58697, 58698, 58699, 58700, 58701, 58702 were selected from the ORFs having the nucleotide sequence represented by SEQ ID NO: 4746.

The probes having the nucleotide sequence represented by SEQ ID NOS: 58703, 58704, 58705, 58706, 58707, 58708, 58709, 58710, 58711, 58712, 58713 were selected from the ORFs having the nucleotide sequence represented by SEQ ID NO: 4747.

The probes having the nucleotide sequence represented by SEQ ID NOS: 58714, 58715, 58716, 58717, 58718, 58719, 58720, 58721, 58722, 58723, 58724 were selected from the ORFs having the nucleotide sequence represented by SEQ ID NO: 4748.

The probes having the nucleotide sequence represented by SEQ ID NOS: 58725, 58726, 58727, 58728, 58729, 58730, 58731, 58732, 58733, 58734, 58735 were selected from the ORFs having the nucleotide sequence represented by SEQ ID NO: 4749.

The probes having the nucleotide sequence represented by SEQ ID NOS: 58736, 58737, 58738, 58739, 58740, 58741, 58742, 58743, 58744, 58745, 58746 were selected from the ORFs having the nucleotide sequence represented by SEQ ID NO: 4750.

The probes having the nucleotide sequence represented by SEQ ID NOS: 58747, 58748, 58749, 58750, 58751, 58752, 58753, 58754, 58755, 58756, 58757 were selected from the ORFs having the nucleotide sequence represented by SEQ ID NO: 4751.

The probes having the nucleotide sequence represented by SEQ ID NOS: 58758, 58759, 58760, 58761, 58762, 58763, 58764, 58765, 58766, 58767, 58768 were selected from the ORFs having the nucleotide sequence represented by SEQ ID NO: 4752.

The probes having the nucleotide sequence represented by SEQ ID NOS: 58769, 58770, 58771, 58772, 58773, 58774, 58775, 58776, 58777, 58778, 58779 were selected from the ORFs having the nucleotide sequence represented by SEQ ID NO: 4753.

The probes having the nucleotide sequence represented by SEQ ID NOS: 58780, 58781, 58782, 58783, 58784, 58785, 58786, 58787, 58788, 58789, 58790 were selected from the ORFs having the nucleotide sequence represented by SEQ ID NO: 4754.

The probes having the nucleotide sequence represented by SEQ ID NOS: 58791, 58792, 58793, 58794, 58795, 58796, 58797, 58798, 58799, 58800, 58801 were selected from the ORFs having the nucleotide sequence represented by SEQ ID NO: 4755.

The probes having the nucleotide sequence represented by SEQ ID NOS: 58802, 58803, 58804, 58805, 58806, 58807, 58808, 58809, 58810, 58811, 58812 were selected from the ORFs having the nucleotide sequence represented by SEQ ID NO: 4756.

The probes having the nucleotide sequence represented by SEQ ID NOS: 58813, 58814, 58815, 58816, 58817, 58818, 58819, 58820, 58821, 58822, 58823 were selected from the ORFs having the nucleotide sequence represented by SEQ ID NO: 4757.

The probes having the nucleotide sequence represented by SEQ ID NOS: 58824, 58825, 58826, 58827, 58828, 58829, 58830, 58831, 58832, 58833, 58834 were selected from the ORFs having the nucleotide sequence represented by SEQ ID NO: 4758.

The probes having the nucleotide sequence represented by SEQ ID NOS: 58835, 58836, 58837, 58838, 58839, 58840, 58841, 58842, 58843, 58844, 58845 were selected from the ORFs having the nucleotide sequence represented by SEQ ID NO: 4759.

The probes having the nucleotide sequence represented by SEQ ID NOS: 58846, 58847, 58848, 58849, 58850, 58851, 58852, 58853, 58854, 58855, 58856 were selected from the ORFs having the nucleotide sequence represented by SEQ ID NO: 4760.

The probes having the nucleotide sequence represented by SEQ ID NOS: 58857, 58858, 58859, 58860, 58861, 58862, 58863, 58864, 58865, 58866, 58867 were selected from the ORFs having the nucleotide sequence represented by SEQ ID NO: 4761.

The probes having the nucleotide sequence represented by SEQ ID NOS: 58868, 58869, 58870, 58871, 58872, 58873, 58874, 58875, 58876, 58877, 58878 were selected from the ORFs having the nucleotide sequence represented by SEQ ID NO: 4762.

The probes having the nucleotide sequence represented by SEQ ID NOS: 58879, 58880, 58881, 58882, 58883, 58884, 58885, 58886, 58887, 58888, 58889 were selected from the ORFs having the nucleotide sequence represented by SEQ ID NO: 4763.

The probes having the nucleotide sequence represented by SEQ ID NOS: 58890, 58891, 58892, 58893, 58894, 58895, 58896, 58897, 58898, 58899, 58900 were selected from the ORFs having the nucleotide sequence represented by SEQ ID NO: 4764.

The probes having the nucleotide sequence represented by SEQ ID NOS: 58901, 58902, 58903, 58904, 58905, 58906, 58907, 58908, 58909, 58910, 58911 were selected from the ORFs having the nucleotide sequence represented by SEQ ID NO: 4765.

The probes having the nucleotide sequence represented by SEQ ID NOS: 58912, 58913, 58914, 58915, 58916, 58917, 58918, 58919, 58920, 58921, 58922 were selected from the ORFs having the nucleotide sequence represented by SEQ ID NO: 4766.

The probes having the nucleotide sequence represented by SEQ ID NOS: 58923, 58924, 58925, 58926, 58927, 58928, 58929, 58930, 58931, 58932, 58933 were selected from the ORFs having the nucleotide sequence represented by SEQ ID NO: 4767.

The probes having the nucleotide sequence represented by SEQ ID NOS: 58934, 58935, 58936, 58937, 58938, 58939, 58940, 58941, 58942, 58943, 58944 were selected from the ORFs having the nucleotide sequence represented by SEQ ID NO: 4768.

The probes having the nucleotide sequence represented by SEQ ID NOS: 58945, 58946, 58947, 58948, 58949, 58950, 58951, 58952, 58953, 58954, 58955 were selected from the ORFs having the nucleotide sequence represented by SEQ ID NO: 4769.

The probes having the nucleotide sequence represented by SEQ ID NOS: 58956, 58957, 58958, 58959, 58960, 58961, 58962, 58963, 58964, 58965, 58966 were selected from the ORFs having the nucleotide sequence represented by SEQ ID NO: 4770.

The probes having the nucleotide sequence represented by SEQ ID NOS: 58967, 58968, 58969, 58970, 58971, 58972, 58973, 58974, 58975, 58976, 58977 were selected from the ORFs having the nucleotide sequence represented by SEQ ID NO: 4771.

The probes having the nucleotide sequence represented by SEQ ID NOS: 58978, 58979, 58980, 58981, 58982, 58983, 58984, 58985, 58986, 58987, 58988 were selected from the ORFs having the nucleotide sequence represented by SEQ ID NO: 4772.

The probes having the nucleotide sequence represented by SEQ ID NOS: 58989, 58990, 58991, 58992, 58993, 58994, 58995, 58996, 58997, 58998, 58999 were selected from the ORFs having the nucleotide sequence represented by SEQ ID NO: 4773.

The probes having the nucleotide sequence represented by SEQ ID NOS: 59000, 59001, 59002, 59003, 59004, 59005, 59006, 59007, 59008, 59009, 59010 were selected from the ORFs having the nucleotide sequence represented by SEQ ID NO: 4774.

The probes having the nucleotide sequence represented by SEQ ID NOS: 59011, 59012, 59013, 59014, 59015, 59016, 59017, 59018, 59019, 59020, 59021 were selected from the ORFs having the nucleotide sequence represented by SEQ ID NO: 4775.

The probes having the nucleotide sequence represented by SEQ ID NOS: 59022, 59023, 59024, 59025, 59026, 59027, 59028, 59029, 59030, 59031, 59032 were selected from the ORFs having the nucleotide sequence represented by SEQ ID NO: 4776.

The probes having the nucleotide sequence represented by SEQ ID NOS: 59033, 59034, 59035, 59036, 59037, 59038, 59039, 59040, 59041, 59042, 59043 were selected from the ORFs having the nucleotide sequence represented by SEQ ID NO: 4777.

The probes having the nucleotide sequence represented by SEQ ID NOS: 59044, 59045, 59046, 59047, 59048, 59049, 59050, 59051, 59052, 59053, 59054 were selected from the ORFs having the nucleotide sequence represented by SEQ ID NO: 4778.

The probes having the nucleotide sequence represented by SEQ ID NOS: 59055, 59056, 59057, 59058, 59059, 59060, 59061, 59062, 59063, 59064, 59065 were selected from the ORFs having the nucleotide sequence represented by SEQ ID NO: 4779.

The probes having the nucleotide sequence represented by SEQ ID NOS: 59066, 59067, 59068, 59069, 59070, 59071, 59072, 59073, 59074, 59075, 59076 were selected from the ORFs having the nucleotide sequence represented by SEQ ID NO: 4780.

The probes having the nucleotide sequence represented by SEQ ID NOS: 59077, 59078, 59079, 59080, 59081, 59082, 59083, 59084, 59085, 59086, 59087 were selected from the ORFs having the nucleotide sequence represented by SEQ ID NO: 4781.

The probes having the nucleotide sequence represented by SEQ ID NOS: 59088, 59089, 59090, 59091, 59092, 59093, 59094, 59095, 59096, 59097, 59098 were selected from the ORFs having the nucleotide sequence represented by SEQ ID NO: 4782.

The probes having the nucleotide sequence represented by SEQ ID NOS: 59099, 59100, 59101, 59102, 59103, 59104, 59105, 59106, 59107, 59108, 59109 were selected from the ORFs having the nucleotide sequence represented by SEQ ID NO: 4783.

The probes having the nucleotide sequence represented by SEQ ID NOS: 59110, 59111, 59112, 59113, 59114, 59115, 59116, 59117, 59118, 59119, 59120 were selected from the ORFs having the nucleotide sequence represented by SEQ ID NO: 4784.

The probes having the nucleotide sequence represented by SEQ ID NOS: 59121, 59122, 59123, 59124, 59125, 59126, 59127, 59128, 59129, 59130, 59131 were selected from the ORFs having the nucleotide sequence represented by SEQ ID NO: 4785.

The probes having the nucleotide sequence represented by SEQ ID NOS: 59132, 59133, 59134, 59135, 59136, 59137, 59138, 59139, 59140, 59141, 59142 were selected from the ORFs having the nucleotide sequence represented by SEQ ID NO: 4786.

The probes having the nucleotide sequence represented by SEQ ID NOS: 59143, 59144, 59145, 59146, 59147, 59148, 59149, 59150, 59151, 59152, 59153 were selected from the ORFs having the nucleotide sequence represented by SEQ ID NO: 4787.

The probes having the nucleotide sequence represented by SEQ ID NOS: 59154, 59155, 59156, 59157, 59158, 59159, 59160, 59161, 59162, 59163, 59164 were selected from the ORFs having the nucleotide sequence represented by SEQ ID NO: 4788.

The probes having the nucleotide sequence represented by SEQ ID NOS: 59165, 59166, 59167, 59168, 59169, 59170, 59171, 59172, 59173, 59174, 59175 were selected from the ORFs having the nucleotide sequence represented by SEQ ID NO: 4789.

The probes having the nucleotide sequence represented by SEQ ID NOS: 59176, 59177, 59178, 59179, 59180, 59181, 59182, 59183, 59184, 59185, 59186 were selected from the ORFs having the nucleotide sequence represented by SEQ ID NO: 4790.

The probes having the nucleotide sequence represented by SEQ ID NOS: 59187, 59188, 59189, 59190, 59191, 59192, 59193, 59194, 59195, 59196, 59197 were selected from the ORFs having the nucleotide sequence represented by SEQ ID NO: 4791.

The probes having the nucleotide sequence represented by SEQ ID NOS: 59198, 59199, 59200, 59201, 59202, 59203, 59204, 59205, 59206, 59207, 59208 were selected from the ORFs having the nucleotide sequence represented by SEQ ID NO: 4792.

The probes having the nucleotide sequence represented by SEQ ID NOS: 59209, 59210, 59211, 59212, 59213, 59214, 59215, 59216, 59217, 59218, 59219 were selected from the ORFs having the nucleotide sequence represented by SEQ ID NO: 4793.

The probes having the nucleotide sequence represented by SEQ ID NOS: 59220, 59221, 59222, 59223, 59224, 59225, 59226, 59227, 59228, 59229, 59230 were selected from the ORFs having the nucleotide sequence represented by SEQ ID NO: 4794.

The probes having the nucleotide sequence represented by SEQ ID NOS: 59231, 59232, 59233, 59234, 59235, 59236, 59237, 59238, 59239, 59240, 59241 were selected from the ORFs having the nucleotide sequence represented by SEQ ID NO: 4795.

The probes having the nucleotide sequence represented by SEQ ID NOS: 59242, 59243, 59244, 59245, 59246, 59247, 59248, 59249, 59250, 59251, 59252 were selected from the ORFs having the nucleotide sequence represented by SEQ ID NO: 4796.

The probes having the nucleotide sequence represented by SEQ ID NOS: 59253, 59254, 59255, 59256, 59257, 59258, 59259, 59260, 59261, 59262, 59263 were selected from the ORFs having the nucleotide sequence represented by SEQ ID NO: 4797.

The probes having the nucleotide sequence represented by SEQ ID NOS: 59264, 59265, 59266, 59267, 59268, 59269, 59270, 59271, 59272, 59273, 59274 were selected from the ORFs having the nucleotide sequence represented by SEQ ID NO: 4798.

The probes having the nucleotide sequence represented by SEQ ID NOS: 59275, 59276, 59277, 59278, 59279, 59280, 59281, 59282, 59283, 59284, 59285 were selected from the ORFs having the nucleotide sequence represented by SEQ ID NO: 4799.

The probes having the nucleotide sequence represented by SEQ ID NOS: 59286, 59287, 59288, 59289, 59290, 59291, 59292, 59293, 59294, 59295, 59296 were selected from the ORFs having the nucleotide sequence represented by SEQ ID NO: 4800.

The probes having the nucleotide sequence represented by SEQ ID NOS: 59297, 59298, 59299, 59300, 59301, 59302, 59303, 59304, 59305, 59306, 59307 were selected from the ORFs having the nucleotide sequence represented by SEQ ID NO: 4801.

The probes having the nucleotide sequence represented by SEQ ID NOS: 59308, 59309, 59310, 59311, 59312, 59313, 59314, 59315, 59316,. 59317, 59318 were selected from the ORFs having the nucleotide sequence represented by SEQ ID NO: 4802.

The probes having the nucleotide sequence represented by SEQ ID NOS: 59319, 59320, 59321, 59322, 59323, 59324, 59325, 59326, 59327, 59328, 59329 were selected from the ORFs having the nucleotide sequence represented by SEQ ID NO: 4803.

The probes having the nucleotide sequence represented by SEQ ID NOS: 59330, 59331, 59332, 59333, 59334, 59335, 59336, 59337, 59338, 59339, 59340 were selected from the ORFs having the nucleotide sequence represented by SEQ ID NO: 4804.

The probes having the nucleotide sequence represented by SEQ ID NOS: 59341, 59342, 59343, 59344, 59345, 59346, 59347, 59348, 59349, 59350, 59351 were selected from the ORFs having the nucleotide sequence represented by SEQ ID NO: 4805.

The probes having the nucleotide sequence represented by SEQ ID NOS: 59352, 59353, 59354, 59355, 59356, 59357, 59358, 59359, 59360, 59361, 59362, 59363, 59364, 59365, 59366, 59367, 59368, 59369, 59370, 59371, 59372, 59373 were selected from the ORFs having the nucleotide sequence represented by SEQ ID NO: 4806.

The probes having the nucleotide sequence represented by SEQ ID NOS: 59374, 59375, 59376, 59377, 59378, 59379, 59380, 59381, 59382, 59383, 59384 were selected from the ORFs having the nucleotide sequence represented by SEQ ID NO: 4807.

The probes having the nucleotide sequence represented by SEQ ID NOS: 59385, 59386, 59387, 59388, 59389, 59390, 59391, 59392, 59393, 59394, 59395 were selected from the ORFs having the nucleotide sequence represented by SEQ ID NO: 4808.

The probes having the nucleotide sequence represented by SEQ ID NOS: 59396, 59397, 59398, 59399, 59400, 59401, 59402, 59403, 59404, 59405, 59406 were selected from the ORFs having the nucleotide sequence represented by SEQ ID NO: 4809.

The probes having the nucleotide sequence represented by SEQ ID NOS: 59407, 59408, 59409, 59410, 59411, 59412, 59413, 59414, 59415, 59416, 59417 were selected from the ORFs having the nucleotide sequence represented by SEQ ID NO: 4810.

The probes having the nucleotide sequence represented by SEQ ID NOS: 59418, 59419, 59420, 59421, 59422, 59423, 59424, 59425, 59426, 59427, 59428 were selected from the ORFs having the nucleotide sequence represented by SEQ ID NO: 4811.

The probes having the nucleotide sequence represented by SEQ ID NOS: 59429, 59430, 59431, 59432, 59433, 59434, 59435, 59436, 59437, 59438, 59439 were selected from the ORFs having the nucleotide sequence represented by SEQ ID NO: 4812.

The probes having the nucleotide sequence represented by SEQ ID NOS: 59440, 59441, 59442, 59443, 59444, 59445, 59446, 59447, 59448, 59449, 59450 were selected from the ORFs having the nucleotide sequence represented by SEQ ID NO: 4813.

The probes having the nucleotide sequence represented by SEQ ID NOS: 59451, 59452, 59453, 59454, 59455, 59456, 59457, 59458, 59459, 59460, 59461 were selected from the ORFs having the nucleotide sequence represented by SEQ ID NO: 4814.

The probes having the nucleotide sequence represented by SEQ ID NOS: 59462, 59463, 59464, 59465, 59466, 59467, 59468, 59469, 59470, 59471, 59472 were selected from the ORFs having the nucleotide sequence represented by SEQ ID NO: 4815.

The probes having the nucleotide sequence represented by SEQ ID NOS: 59473, 59474, 59475, 59476, 59477, 59478, 59479, 59480, 59481, 59482, 59483 were selected from the ORFs having the nucleotide sequence represented by SEQ ID NO: 4816.

The probes having the nucleotide sequence represented by SEQ ID NOS: 59484, 59485, 59486, 59487, 59488, 59489, 59490, 59491, 59492, 59493, 59494 were selected from the ORFs having the nucleotide sequence represented by SEQ ID NO: 4817.

The probes having the nucleotide sequence represented by SEQ ID NOS: 59495, 59496, 59497, 59498, 59499, 59500, 59501, 59502, 59503, 59504, 59505 were selected from the ORFs having the nucleotide sequence represented by SEQ ID NO: 4818.

The probes having the nucleotide sequence represented by SEQ ID NOS: 59506, 59507, 59508, 59509, 59510, 59511, 59512, 59513, 59514, 59515, 59516 were selected from the ORFs having the nucleotide sequence represented by SEQ ID NO: 4819.

The probes having the nucleotide sequence represented by SEQ ID NOS: 59517, 59518, 59519, 59520, 59521, 59522, 59523, 59524, 59525, 59526, 59527 were selected from the ORFs having the nucleotide sequence represented by SEQ ID NO: 4820.

The probes having the nucleotide sequence represented by SEQ ID NOS: 59528, 59529, 59530, 59531, 59532, 59533, 59534, 59535, 59536, 59537, 59538 were selected from the ORFs having the nucleotide sequence represented by SEQ ID NO: 4821.

The probes having the nucleotide sequence represented by SEQ ID NOS: 59539, 59540, 59541, 59542, 59543, 59544, 59545, 59546, 59547, 59548, 59549 were selected from the ORFs having the nucleotide sequence represented by SEQ ID NO: 4822.

The probes having the nucleotide sequence represented by SEQ ID NOS: 59550, 59551, 59552, 59553, 59554, 59555, 59556, 59557, 59558, 59559, 59560 were selected from the ORFs having the nucleotide sequence represented by SEQ ID NO: 4823.

The probes having the nucleotide sequence represented by SEQ ID NOS: 59561, 59562, 59563, 59564, 59565, 59566, 59567, 59568, 59569, 59570, 59571 were selected from the ORFs having the nucleotide sequence represented by SEQ ID NO: 4824.

The probes having the nucleotide sequence represented by SEQ ID NOS: 59572, 59573, 59574, 59575, 59576, 59577, 59578, 59579, 59580, 59581, 59582 were selected from the ORFs having the nucleotide sequence represented by SEQ ID NO: 4825.

The probes having the nucleotide sequence represented by SEQ ID NOS: 59583, 59584, 59585, 59586, 59587, 59588, 59589, 59590, 59591, 59592, 59593 were selected from the ORFs having the nucleotide sequence represented by SEQ ID NO: 4826.

The probes having the nucleotide sequence represented by SEQ ID NOS: 59594, 59595, 59596, 59597, 59598, 59599, 59600, 59601, 59602, 59603, 59604 were selected from the ORFs having the nucleotide sequence represented by SEQ ID NO: 4827.

The probes having the nucleotide sequence represented by SEQ ID NOS: 59605, 59606, 59607, 59608, 59609, 59610, 59611, 59612, 59613, 59614, 59615, 59616, 59617, 59618, 59619, 59620, 59621, 59622, 59623, 59624, 59625, 59626 were selected from the ORFs having the nucleotide sequence represented by SEQ ID NO: 4828.

The probes having the nucleotide sequence represented by SEQ ID NOS: 59627, 59628, 59629, 59630, 59631, 59632, 59633, 59634, 59635, 59636, 59637 were selected from the ORFs having the nucleotide sequence represented by SEQ ID NO: 4829.

The probes having the nucleotide sequence represented by SEQ ID NOS: 59638, 59639, 59640, 59641, 59642, 59643, 9644, 59645, 59646, 59647, 59648 were selected from the ORFs having the nucleotide sequence represented by SEQ ID NO: 4830.

The probes having the nucleotide sequence represented by SEQ ID NOS: 59649, 59650, 59651, 59652, 59653, 59654, 59655, 59656, 59657, 59658, 59659 were selected from the ORFs having the nucleotide sequence represented by SEQ ID NO: 4831.

The probes having the nucleotide sequence represented by SEQ ID NOS: 59660, 59661, 59662, 59663, 59664, 59665, 59666, 59667, 59668, 59669, 59670 were selected from the ORFs having the nucleotide sequence represented by SEQ ID NO: 4832.

The probes having the nucleotide sequence represented by SEQ ID NOS: 59671, 59672, 59673, 59674, 59675, 59676, 59677, 59678, 59679, 59680, 59681 were selected from the ORFs having the nucleotide sequence represented by SEQ ID NO: 4833.

The probes having the nucleotide sequence represented by SEQ ID NOS: 59682, 59683, 59684, 59685, 59686, 59687, 59688, 59689, 59690, 59691, 59692 were selected from the ORFs having the nucleotide sequence represented by SEQ ID NO: 4834.

The probes having-the nucleotide sequence represented by SEQ ID NOS: 59693, 59694, 59695, 59696, 59697, 59698, 59699, 59700, 59701, 59702, 59703 were selected from the ORFs having the nucleotide sequence represented by SEQ ID NO: 4835.

The probes having the nucleotide sequence represented by SEQ ID NOS: 59704, 59705, 59706, 59707, 59708, 59709, 59710, 59711, 59712, 59713, 59714 were selected from the ORFs having the nucleotide sequence represented by SEQ ID NO: 4836.

The probes having the nucleotide sequence represented by SEQ ID NOS: 59715, 59716, 59717, 59718, 59719, 59720, 59721, 59722, 59723, 59724, 59725 were selected from the ORFs having the nucleotide sequence represented by SEQ ID NO: 4837.

The probes having the nucleotide sequence represented by SEQ ID NOS: 59726, 59727, 59728, 59729, 59730, 59731, 59732, 59733, 59734, 59735, 59736 were selected from the ORFs having the nucleotide sequence represented by SEQ ID NO: 4838.

The probes having the nucleotide sequence represented by SEQ ID NOS: 59737, 59738, 59739, 59740, 59741, 59742, 59743, 59744, 59745, 59746, 59747 were selected from the ORFs having the nucleotide sequence represented by SEQ ID NO: 4839.

The probes having the nucleotide sequence represented by SEQ ID NOS: 59748, 59749, 59750, 59751, 59752, 59753, 59754, 59755, 59756, 59757, 59758 were selected from the ORFs having the nucleotide sequence represented by SEQ ID NO: 4840.

The probes having the nucleotide sequence represented by SEQ ID NOS: 59759, 59760, 59761, 59762, 59763, 59764, 59765, 59766, 59767, 59768, 59769 were selected from the ORFs having the nucleotide sequence represented by SEQ ID NO: 4841.

The probes having the nucleotide sequence represented by SEQ ID-NOS: 59770, 59771, 59772, 59773, 59774, 59775, 59776, 59777, 59778, 59779, 59780 were selected from the ORFs having the nucleotide sequence represented by SEQ ID NO: 4842.

The probes having the nucleotide sequence represented by SEQ ID NOS: 59781, 59782, 59783, 59784, 59785, 59786, 59787, 59788, 59789, 59790, 59791 were selected from the ORFs having the nucleotide sequence represented by SEQ ID NO: 4843.

The probes having the nucleotide sequence represented by SEQ ID NOS: 59792, 59793, 59794, 59795, 59796, 59797, 59798, 59799, 59800, 59801, 59802 were selected from the ORFs having the nucleotide sequence represented by SEQ ID NO: 4844.

The probes having the nucleotide sequence represented by SEQ ID NOS: 59803, 59804, 59805, 59806, 59807, 59808, 59809, 59810, 59811, 59812, 59813 were selected from the ORFs having the nucleotide sequence represented by SEQ ID NO: 4845.

The probes having the nucleotide sequence represented by SEQ ID NOS: 59814, 59815, 59816, 59817, 59818, 59819, 59820, 59821, 59822, 59823, 59824 were selected from the ORFs having the nucleotide sequence represented by SEQ ID NO: 4846.

The probes having the nucleotide sequence represented by SEQ ID NOS: 59825, 59826, 59827, 59828, 59829, 59830, 59831, 59832, 59833, 59834, 59835 were selected from the ORFs having the nucleotide sequence represented by SEQ ID NO: 4847.

The probes having the nucleotide sequence represented by SEQ ID NOS: 59836, 59837, 59838, 59839, 59840, 59841, 59842, 59843, 59844, 59845, 59846 were selected from the ORFs having the nucleotide sequence represented by SEQ ID NO: 4848.

The probes having the nucleotide sequence represented by SEQ ID NOS: 59847, 59848, 59849, 59850, 59851, 59852, 59853, 59854, 59855, 59856, 59857 were selected from the ORFs having the nucleotide sequence represented by SEQ ID NO: 4849.

The probes having the nucleotide sequence represented by SEQ ID NOS: 59858, 59859, 59860, 59861, 59862, 59863, 59864, 59865, 59866, 59867, 59868 were selected from the ORFs having the nucleotide sequence represented by SEQ ID NO: 4850.

The probes having the nucleotide sequence represented by SEQ ID NOS: 59869, 59870, 59871, 59872, 59873, 59874, 59875, 59876, 5.9877, 59878, 59879 were selected from the ORFs having the nucleotide sequence represented by SEQ ID NO: 4851.

The probes having the nucleotide sequence represented by SEQ ID NOS: 59880, 59881, 59882, 59883, 59884, 59885, 59886, 59887, 59888, 59889, 59890 were selected from the ORFs having the nucleotide sequence represented by SEQ ID NO: 4852.

The probes having the nucleotide sequence represented by SEQ ID NOS: 59891, 59892, 59893, 59894, 59895, 59896, 59897, 59898, 59899, 59900, 59901 were selected from the ORFs having the nucleotide sequence represented by SEQ ID NO: 4853.

The probes having the nucleotide sequence represented by SEQ ID NOS: 59902, 59903, 59904, 59905, 59906, 59907, 59908, 59909, 59910, 59911, 59912 were selected from the ORFs having the nucleotide sequence represented by SEQ ID NO: 4854.

The probes having the nucleotide sequence represented by SEQ ID NOS: 59913, 59914, 59915, 59916, 59917, 59918, 59919, 59920, 59921, 59922, 59923 were selected from the ORFs having the nucleotide sequence represented by SEQ ID NO: 4855.

The probes having the nucleotide sequence represented by SEQ ID NOS: 59924, 59925, 59926, 59927, 59928, 59929, 59930, 59931, 59932, 59933, 59934 were selected from the ORFs having the nucleotide sequence represented by SEQ ID NO: 4856.

The probes having the nucleotide sequence represented by SEQ ID NOS: 59935, 59936, 59937, 59938, 59939, 59940, 59941, 59942, 59943, 59944, 59945 were selected from the ORFs having the nucleotide sequence represented by SEQ ID NO: 4857.

The probes having the nucleotide sequence represented by SEQ ID NOS: 59946, 59947, 59948, 59949, 59950, 59951, 59952, 59953, 59954, 59955, 59956 were selected from the ORFs having the nucleotide sequence represented by SEQ ID NO: 4858.

The probes having the nucleotide sequence represented by SEQ ID NOS: 59957, 59958, 59959, 59960, 59961, 59962, 59963, 59964, 59965, 59966, 59967 were selected from the ORFs having the nucleotide sequence represented by SEQ ID NO: 4859.

The probes having the nucleotide sequence represented by SEQ ID NOS: 59968, 59969, 59970, 59971, 59972, 59973, 59974, 59975, 59976, 59977, 59978 were selected from the ORFs having the nucleotide sequence represented by SEQ ID NO: 4860.

The probes having the nucleotide sequence represented by SEQ ID NOS: 59979, 59980, 59981, 59982, 59983, 59984, 59985, 59986, 59987, 59988, 59989 were selected from the ORFs having the nucleotide sequence represented by SEQ ID NO: 4861.

The probes having the nucleotide sequence represented by SEQ ID NOS: 59990, 59991, 59992, 59993, 59994, 59995, 59996, 59997, 59998, 59999, 60000 were selected from the ORFs having the nucleotide sequence represented by SEQ ID NO: 4862.

The probes having the nucleotide sequence represented by SEQ ID NOS: 60001, 60002, 60003, 60004, 60005, 60006, 60007, 60008, 60009, 60010, 60011 were selected from the ORFs having the nucleotide sequence represented by SEQ ID NO: 4863.

The probes having the nucleotide sequence represented by SEQ ID NOS: 60012, 60013, 60014, 60015, 60016, 60017, 60018, 60019, 60020, 60021, 60022 were selected from the ORFs having the nucleotide sequence represented by SEQ ID NO: 4864.

The probes having the nucleotide sequence represented by SEQ ID NOS: 60023, 60024, 60025, 60026, 60027, 60028, 60029, 60030, 60031, 60032, 60033 were selected from the ORFs having the nucleotide sequence represented by SEQ ID NO: 4865.

The probes having the nucleotide sequence represented by SEQ ID NOS: 60034, 60035, 60036, 60037, 60038, 60039, 60040, 60041, 60042, 60043, 60044 were selected from the ORFs having the nucleotide sequence represented by SEQ ID NO: 4866.

The probes having the nucleotide sequence represented by SEQ ID NOS: 60045, 60046, 60047, 60048, 60049, 60050, 60051, 60052, 60053, 60054, 60055 were selected from the ORFs having the nucleotide sequence represented by SEQ ID NO: 4867.

The probes having the nucleotide sequence represented by SEQ ID NOS: 60056, 60057, 60058, 60059, 60060, 60061, 60062, 60063, 60064, 60065, 60066 were selected from the ORFs having the nucleotide sequence represented by SEQ ID NO: 4868.

The probes having the nucleotide sequence represented by SEQ ID NOS: 60067, 60068, 60069, 60070, 60071, 60072, 60073, 60074, 60075, 60076, 60077 were selected from the ORFs having the nucleotide sequence represented by SEQ ID NO: 4869.

The probes having the nucleotide sequence represented by SEQ ID NOS: 60078, 60079, 60080, 60081, 60082, 60083, 60084, 60085, 60086, 60087, 60088 were selected from the ORFs having the nucleotide sequence represented by SEQ ID NO: 4870.

The probes having the nucleotide sequence represented by SEQ ID NOS: 60089, 60090, 60091, 60092, 60093, 60094, 60095, 60096, 60097, 60098, 60099 were selected from the ORFs having the nucleotide sequence represented by SEQ ID NO: 4871.

The probes having the nucleotide sequence represented by SEQ ID NOS: 60100, 60101, 60102, 60103, 60104, 60105, 60106, 60107, 60108, 60109, 60110 were selected from the ORFs having the nucleotide sequence represented by SEQ ID NO: 4872.

The probes having the nucleotide sequence represented by SEQ ID NOS: 60111, 60112, 60113, 60114, 60115, 60116, 60117, 60118, 60119, 60120, 60121 were selected from the ORFs having the nucleotide sequence represented by SEQ ID NO: 4873.

The probes having the nucleotide sequence represented by SEQ ID NOS: 60122, 60123, 60124, 60125, 60126, 60127, 60128, 60129, 60130, 60131, 60132 were selected from the ORFs having the nucleotide sequence represented by SEQ ID NO: 4874.

The probes having the nucleotide sequence represented by SEQ ID NOS: 60133, 60134, 60135, 60136, 60137, 60138, 60139, 60140, 60141, 60142, 60143 were selected from the ORFs having the nucleotide sequence represented by SEQ ID NO: 4875.

The probes having the nucleotide sequence represented by SEQ ID NOS: 60144, 60145, 60146, 60147, 60148, 60149, 60150, 60151, 60152, 60153, 60154 were selected from the ORFs having the nucleotide sequence represented by SEQ ID NO: 4876.

The probes having the nucleotide sequence represented by SEQ ID NOS: 60155, 60156, 60157, 60158, 60159, 60160, 60161, 60162, 60163, 60164, 60165 were selected from the ORFs having the nucleotide sequence represented by SEQ ID NO: 4877.

The probes having the nucleotide sequence represented by SEQ ID NOS: 60166, 60167, 60168, 60169, 60170, 60171, 60172, 60173, 60174, 60175, 60176 were selected from the ORFs having the nucleotide sequence represented by SEQ ID NO: 4878.

The probes having the nucleotide sequence represented by SEQ ID NOS: 60177, 60178, 60179, 60180, 60181, 60182, 60183, 60184, 60185, 60186, 60187 were selected from the ORFs having the nucleotide sequence represented by SEQ ID NO: 4879.

The probes having the nucleotide sequence represented by SEQ ID NOS: 60188, 60189, 60190, 60191, 60192, 60193, 60194, 60195, 60196, 60197, 60198 were selected from the ORFs having the nucleotide sequence represented by SEQ ID NO: 4880.

The probes having the nucleotide sequence represented by SEQ ID NOS: 60199, 60200, 60201, 60202, 60203, 60204, 60205, 60206, 60207, 60208, 60209 were selected from the ORFs having the nucleotide sequence represented by SEQ ID NO: 4881.

The probes having the nucleotide sequence represented by SEQ ID NOS: 60210, 60211, 60212, 60213, 60214, 60215, 60216, 60217, 60218, 60219, 60220 were selected from the ORFs having the nucleotide sequence represented by SEQ ID NO: 4882.

The probes having the nucleotide sequence represented by SEQ ID NOS: 60221, 60222, 60223, 60224, 60225, 60226, 60227, 60228, 60229, 60230, 60231 were selected from the ORFs having the nucleotide sequence represented by SEQ ID NO: 4883.

The probes having the nucleotide sequence represented by SEQ ID NOS: 60232, 60233, 60234, 60235, 60236, 60237, 60238, 60239, 60240, 60241, 60242 were selected from the ORFs having the nucleotide sequence represented by SEQ ID NO: 4884.

The probes having the nucleotide sequence represented by SEQ ID NOS: 60243, 60244, 60245, 60246, 60247, 60248, 60249, 60250, 60251, 60252, 60253 were selected from the ORFs having the nucleotide sequence represented by SEQ ID NO: 4885.

The probes having the nucleotide sequence represented by SEQ ID NOS: 60254, 60255, 60256, 60257, 60258, 60259, 60260, 60261, 60262, 60263, 60264 were selected from the ORFs having the nucleotide sequence represented by SEQ ID NO: 4886.

The probes having the nucleotide sequence represented by SEQ ID NOS: 60265, 60266, 60267, 60268, 60269, 60270, 60271, 60272, 60273, 60274, 60275 were selected from the ORFs having the nucleotide sequence represented by SEQ ID NO: 4887.

The probes having the nucleotide sequence represented by SEQ ID NOS: 60276, 60277, 60278, 60279, 60280, 60281, 60282, 60283, 60284, 60285, 60286 were selected from the ORFs having the nucleotide sequence represented by SEQ ID NO: 4888.

The probes having the nucleotide sequence represented by SEQ ID NOS: 60287, 60288, 60289, 60290, 60291, 60292, 60293, 60294, 60295, 60296, 60297 were selected from the ORFs having the nucleotide sequence represented by SEQ ID NO: 4889.

The probes having the nucleotide sequence represented by SEQ ID NOS: 60298, 60299, 60300, 60301, 60302, 60303, 60304, 60305, 60306, 60307, 60308 were selected from the ORFs having the nucleotide sequence represented by SEQ ID NO: 4890.

The probes having the nucleotide sequence represented by SEQ ID NOS: 60309, 60310, 60311, 60312, 60313, 60314, 60315, 60316, 60317, 60318, 60319 were selected from the ORFs having the nucleotide sequence represented by SEQ ID NO: 4891.

The probes having the nucleotide sequence represented by SEQ ID NOS: 60320, 60321, 60322, 60323, 60324, 60325, 60326, 60327, 60328, 60329, 60330 were selected from the ORFs having the nucleotide sequence represented by SEQ ID NO: 4892.

The probes having the nucleotide sequence represented by SEQ ID NOS: 60331, 60332, 60333, 60334, 60335, 60336, 60337, 60338, 60339, 60340, 60341 were selected from the ORFs having the nucleotide sequence represented by SEQ ID NO: 4893.

The probes having the nucleotide sequence represented by SEQ ID NOS: 60342, 60343, 60344, 60345, 60346, 60347, 60348, 60349, 60350, 60351, 60352 were selected from the ORFs having the nucleotide sequence represented by SEQ ID NO: 4894.

The probes having the nucleotide sequence represented by SEQ ID NOS: 60353, 60354, 60355, 60356, 60357, 60358, 60359, 60360, 60361, 60362, 60363 were selected from the ORFs having the nucleotide sequence represented by SEQ ID NO: 4895.

The probes having the nucleotide sequence represented by SEQ ID NOS: 60364, 60365, 60366, 60367, 60368, 60369, 60370, 60371, 60372, 60373, 60374 were selected from the ORFs having the nucleotide sequence represented by SEQ ID NO: 4896.

The probes having the nucleotide sequence represented by SEQ ID NOS: 60375, 60376, 60377, 60378, 60379, 60380, 60381, 60382, 60383, 60384, 60385 were selected from the ORFs having the nucleotide sequence represented by SEQ ID NO: 4897.

The probes having the nucleotide sequence represented by SEQ ID NOS: 60386, 60387, 60388, 60389, 60390, 60391, 60392, 60393, 60394, 60395, 60396 were selected from the ORFs having the nucleotide sequence represented by SEQ ID NO: 4898.

The probes having the nucleotide sequence represented by SEQ ID NOS: 60397, 60398, 60399, 60400, 60401, 60402, 60403, 60404, 60405, 60406, 60407 were selected from the ORFs having the nucleotide sequence represented by SEQ ID NO: 4899.

The probes having the nucleotide sequence represented by SEQ ID NOS: 60408, 60409, 60410, 60411, 60412, 60413, 60414, 60415, 60416, 60417, 60418 were selected from the ORFs having the nucleotide sequence represented by SEQ ID NO: 4900.

The probes having the nucleotide sequence represented by SEQ ID NOS: 60419, 60420, 60421, 60422, 60423, 60424, 60425, 60426, 60427, 60428, 60429 were selected from the ORFs having the nucleotide sequence represented by SEQ ID NO: 4901.

The probes having the nucleotide sequence represented by SEQ ID NOS: 60430, 60431, 60432, 60433, 60434, 60435, 60436, 60437, 60438, 60439, 60440 were selected from the ORFs having the nucleotide sequence represented by SEQ ID NO: 4902.

The probes having the nucleotide sequence represented by SEQ ID NOS: 60441, 60442, 60443, 60444, 60445, 60446, 60447, 60448, 60449, 60450, 60451 were selected from the ORFs having the nucleotide sequence represented by SEQ ID NO: 4903.

The probes having the nucleotide sequence represented by SEQ ID NOS: 60452, 60453, 60454, 60455, 60456, 60457, 60458, 60459, 60460, 60461, 60462 were selected from the ORFs having the nucleotide sequence represented by SEQ ID NO: 4904.

The probes having the nucleotide sequence represented by SEQ ID NOS: 60463, 60464, 60465, 60466, 60467, 60468, 60469, 60470, 60471, 60472, 60473 were selected from the ORFs having the nucleotide sequence represented by SEQ ID NO: 4905.

The probes having the nucleotide sequence represented by SEQ ID NOS: 60474, 60475, 60476, 60477, 60478, 60479, 60480, 60481, 60482, 60483, 60484 were selected from the ORFs having the nucleotide sequence represented by SEQ ID NO: 4906.

The probes having the nucleotide sequence represented by SEQ ID NOS: 60485, 60486, 60487, 60488, 60489, 60490, 60491, 60492, 60493, 60494, 60495 were selected from the ORFs having the nucleotide sequence represented by SEQ ID NO: 4907.

The probes having the nucleotide sequence represented by SEQ ID NOS: 60496, 60497, 60498, 60499, 60500, 60501, 60502, 60503, 60504, 60505, 60506 were selected from the ORFs having the nucleotide sequence represented by SEQ ID NO: 4908.

The probes having the nucleotide sequence represented by SEQ ID NOS: 60507, 60508, 60509, 60510, 60511, 60512, 60513, 60514, 60515, 60516, 60517 were selected from the ORFs having the nucleotide sequence represented by SEQ ID NO: 4909.

The probes having the nucleotide sequence represented by SEQ ID NOS: 60518, 60519, 60520, 60521, 60522, 60523, 60524, 60525, 60526, 60527, 60528 were selected from the ORFs having the nucleotide sequence represented by SEQ ID NO: 4910.

The probes having the nucleotide sequence represented by SEQ ID NOS: 60529, 60530, 60531, 60532, 60533, 60534, 60535, 60536, 60537, 60538, 60539 were selected from the ORFs having the nucleotide sequence represented by SEQ ID NO: 4911.

The probes having the nucleotide sequence represented by SEQ ID NOS: 60540, 60541, 60542, 60543, 60544, 60545, 60546, 60547, 60548, 60549, 60550 were selected from the ORFs having the nucleotide sequence represented by SEQ ID NO: 4912.

The probes having the nucleotide sequence represented by SEQ ID NOS: 60551, 60552, 60553, 60554, 60555, 60556, 60557, 60558, 60559, 60560, 60561 were selected from the ORFs having the nucleotide sequence represented by SEQ ID NO: 4913.

The probes having the nucleotide sequence represented by SEQ ID NOS: 60562, 60563, 60564, 60565, 60566, 60567, 60568, 60569, 60570, 60571, 60572 were selected from the ORFs having the nucleotide sequence represented by SEQ ID NO: 4914.

The probes having the nucleotide sequence represented by SEQ ID NOS: 60573, 60574, 60575, 60576, 60577, 60578, 60579, 60580, 60581, 60582, 60583 were selected from the ORFs having the nucleotide sequence represented by SEQ ID NO: 4915.

The probes having the nucleotide sequence represented by SEQ ID NOS: 60584, 60585, 60586, 60587, 60588, 60589, 60590, 60591, 60592, 60593, 60594 were selected from the ORFs having the nucleotide sequence represented by SEQ ID NO: 4916.

The probes having the nucleotide sequence represented by SEQ ID NOS: 60595, 60596, 60597, 60598, 60599, 60600, 60601, 60602, 60603, 60604, 60605 were selected from the ORFs having the nucleotide sequence represented by SEQ ID NO: 4917.

The probes having the nucleotide sequence represented by SEQ ID NOS: 60606, 60607, 60608, 60609, 60610, 60611, 60612, 60613, 60614, 60615, 60616 were selected from the ORFs having the nucleotide sequence represented by SEQ ID NO: 4918.

The probes having the nucleotide sequence represented by SEQ ID NOS: 60617, 60618, 60619, 60620, 60621, 60622, 60623, 60624, 60625, 60626, 60627 were selected from the ORFs having the nucleotide sequence represented by SEQ ID NO: 4919.

The probes having the nucleotide sequence represented by SEQ ID NOS: 60628, 60629, 60630, 60631, 60632, 60633, 60634, 60635, 60636, 60637, 60638 were selected from the ORFs having the nucleotide sequence represented by SEQ ID NO: 4920.

The probes having the nucleotide sequence represented by SEQ ID NOS: 60639, 60640, 60641, 60642, 60643, 60644, 60645, 60646, 60647, 60648, 60649 were selected from the ORFs having the nucleotide sequence represented by SEQ ID NO: 4921.

The probes having the nucleotide sequence represented by SEQ ID NOS: 60650, 60651, 60652, 60653, 60654, 60655, 60656, 60657, 60658, 60659, 60660 were selected from the ORFs having the nucleotide sequence represented by SEQ ID NO: 4922.

The probes having the nucleotide sequence represented by SEQ ID NOS: 60661, 60662, 60663, 60664, 60665, 60666, 60667, 60668, 60669, 60670, 60671 were selected from the ORFs having the nucleotide sequence represented by SEQ ID NO: 4923.

The probes having the nucleotide sequence represented by SEQ ID NOS: 60672, 60673, 60674, 60675, 60676, 60677, 60678, 60679, 60680, 60681, 60682 were selected from the ORFs having the nucleotide sequence represented by SEQ ID NO: 4924.

The probes having the nucleotide sequence represented by SEQ ID NOS: 60683, 60684, 60685, 60686, 60687, 60688, 60689, 60690, 60691, 60692, 60693 were selected from the ORFs having the nucleotide sequence represented by SEQ ID NO: 4925.

The probes having the nucleotide sequence represented by SEQ ID NOS: 60694, 60695, 60696, 60697, 60698, 60699, 60700, 60701, 60702, 60703, 60704 were selected from the ORFs having the nucleotide sequence represented by SEQ ID NO: 4926.

The probes having the nucleotide sequence represented by SEQ ID NOS: 60705, 60706, 60707, 60708, 60709, 60710, 60711, 60712, 60713, 60714, 60715 were selected from the ORFs having the nucleotide sequence represented by SEQ ID NO: 4927.

The probes having the nucleotide sequence represented by SEQ ID NOS: 60716, 60717, 60718, 60719, 60720, 60721, 60722, 60723, 60724, 60725, 60726 were selected from the ORFs having the nucleotide sequence represented by SEQ ID NO: 4928.

The probes having the nucleotide sequence represented by SEQ ID NOS: 60727, 60728, 60729, 60730, 60731, 60732, 60733, 60734, 60735, 60736, 60737 were selected from the ORFs having the nucleotide sequence represented by SEQ ID NO: 4929.

The probes having the nucleotide sequence represented by SEQ ID NOS: 60738, 60739, 60740, 60741, 60742, 60743, 60744, 60745, 60746, 60747, 60748 were selected from the ORFs having the nucleotide sequence represented by SEQ ID NO: 4930.

The probes having the nucleotide sequence represented by SEQ ID NOS: 60749, 60750, 60751, 60752, 60753, 60754, 60755, 60756, 60757, 60758, 60759 were selected from the ORFs having the nucleotide sequence represented by SEQ ID NO: 4931.

The probes having the nucleotide sequence represented by SEQ ID NOS: 60760, 60761, 60762, 60763, 60764, 60765, 60766, 60767, 60768, 60769, 60770 were selected from the ORFs having the nucleotide sequence represented by SEQ ID NO: 4932.

The probes having the nucleotide sequence represented by SEQ ID NOS: 60771, 60772, 60773, 60774, 60775, 60776, 60777, 60778, 60779, 60780, 60781 were selected from the ORFs having the nucleotide sequence represented by SEQ ID NO: 4933.

The probes having the nucleotide sequence represented by SEQ ID NOS: 60782, 60783, 60784, 60785, 60786, 60787, 60788, 60789, 60790, 60791, 60792 were selected from the ORFs having the nucleotide sequence represented by SEQ ID NO: 4934.

The probes having the nucleotide sequence represented by SEQ ID NOS: 60793, 60794, 60795, 60796, 60797, 60798, 60799, 60800, 60801, 60802, 60803 were selected from the ORFs having the nucleotide sequence represented by SEQ ID NO: 4935.

The probes having the nucleotide sequence represented by SEQ ID NOS: 60804, 60805, 60806, 60807, 60808, 60809, 60810, 60811, 60812, 60813, 60814 were selected from the ORFs having the nucleotide sequence represented by SEQ ID NO: 4936.

The probes having the nucleotide sequence represented by SEQ ID NOS: 60815, 60816, 60817, 60818, 60819, 60820, 60821, 60822, 60823, 60824, 60825 were selected from the ORFs having the nucleotide sequence represented by SEQ ID NO: 4937.

The probes having the nucleotide sequence represented by SEQ ID NOS: 60826, 60827, 60828, 60829, 60830, 60831, 60832, 60833, 60834, 60835, 60836 were selected from the ORFs having the nucleotide sequence represented by SEQ ID NO: 4938.

The probes having the nucleotide sequence represented by SEQ ID NOS: 60837, 60838, 60839, 60840, 60841, 60842, 60843, 60844, 60845, 60846, 60847 were selected from the ORFs having the nucleotide sequence represented by SEQ ID NO: 4939.

The probes having the nucleotide sequence represented by SEQ ID NOS: 60848, 60849, 60850, 60851, 60852, 60853, 60854, 60855, 60856, 60857, 60858 were selected from the ORFs having the nucleotide sequence represented by SEQ ID NO: 4940.

The probes having the nucleotide sequence represented by SEQ ID NOS: 60859, 60860, 60861, 60862, 60863, 60864, 60865, 60866, 60867, 60868, 60869 were selected from the ORFs having the nucleotide sequence represented by SEQ ID NO: 4941.

The probes having the nucleotide sequence represented by SEQ ID NOS: 60870, 60871, 60872, 60873, 60874, 60875, 60876, 60877, 60878, 60879, 60880 were selected from the ORFs having the nucleotide sequence represented by SEQ ID NO: 4942.

The probes having the nucleotide sequence represented by SEQ ID NOS: 60881, 60882, 60883, 60884, 60885, 60886, 60887, 60888, 60889, 60890, 60891 were selected from the ORFs having the nucleotide sequence represented by SEQ ID NO: 4943.

The probes having the nucleotide sequence represented by SEQ ID NOS: 60892, 60893, 60894, 60895, 60896, 60897, 60898, 60899, 60900, 60901, 60902 were selected from the ORFs having the nucleotide sequence represented by SEQ ID NO: 4944.

The probes having the nucleotide sequence represented by SEQ ID NOS: 60903, 60904, 60905, 60906, 60907, 60908, 60909, 60910, 60911, 60912, 60913 were selected from the ORFs having the nucleotide sequence represented by SEQ ID NO: 4945.

The probes having the nucleotide sequence represented by SEQ ID NOS: 60914, 60915, 60916, 60917, 60918, 60919, 60920, 60921, 60922, 60923, 60924 were selected from the ORFs having the nucleotide sequence represented by SEQ ID NO: 4946.

The probes having the nucleotide sequence represented by SEQ ID NOS: 60925, 60926, 60927, 60928, 60929, 60930, 60931, 60932, 60933, 60934, 60935 were selected from the ORFs having the nucleotide sequence represented by SEQ ID NO: 4947.

The probes having the nucleotide sequence represented by SEQ ID NOS: 60936, 60937, 60938, 60939, 60940, 60941, 60942, 60943, 60944, 60945, 60946 were selected from the ORFs having the nucleotide sequence represented by SEQ ID NO: 4948.

The probes having the nucleotide sequence represented by SEQ ID NOS: 60947, 60948, 60949, 60950, 60951, 60952, 60953, 60954, 60955, 60956, 60957 were selected from the ORFs having the nucleotide sequence represented by SEQ ID NO: 4949.

The probes having the nucleotide sequence represented by SEQ ID NOS:-60958, 60959, 60960, 60961, 60962, 60963, 60964, 60965, 60966, 60967, 60968 were selected from the ORFs having the nucleotide sequence represented by SEQ ID NO: 4950.

The probes having the nucleotide sequence represented by SEQ ID NOS: 60969, 60970, 60971, 60972, 60973, 60974, 60975, 60976, 60977, 60978, 60979 were selected from the ORFs having the nucleotide sequence represented by SEQ ID NO: 4951.

The probes having the nucleotide sequence represented by SEQ ID NOS: 60980, 60981, 60982, 60983, 60984, 60985, 60986, 60987, 60988, 60989, 60990 were selected from the ORFs having the nucleotide sequence represented by SEQ ID- NO: 4952.

The probes having the nucleotide sequence represented by SEQ ID NOS: 60991, 60992, 60993, 60994, 60995, 60996, 60997, 60998, 60999, 61000, 61001 were selected from the ORFs having the nucleotide sequence represented by SEQ ID NO: 4953.

The probes having the nucleotide sequence represented by SEQ ID NOS: 61002, 61003, 61004, 61005, 61006, 61007, 61008, 61009, 61010, 61011, 61012 were selected from the ORFs having the nucleotide sequence represented by SEQ ID NO: 4954.

The probes having the nucleotide sequence represented by SEQ ID NOS: 61013, 61014, 61015, 61016, 61017, 61018, 61019, 61020, 61021, 61022, 61023 were selected from the ORFs having the nucleotide sequence represented by SEQ ID NO: 4955.

The probes having the nucleotide sequence represented by SEQ ID NOS: 61024, 61025, 61026, 61027, 61028, 61029, 61030, 61031, 61032, 61033, 61034 were selected from the ORFs having the nucleotide sequence represented by SEQ ID NO: 4956.

The probes having the nucleotide sequence represented by SEQ ID NOS: 61035, 61036, 61037, 61038, 61039, 61040, 61041, 61042, 61043, 61044, 61045 were selected from the ORFs having the nucleotide sequence represented by SEQ ID NO: 4957.

The probes having the nucleotide sequence represented by SEQ ID NOS: 61046, 61047, 61048, 61049, 61050, 61051, 61052, 61053, 61054, 61055, 61056 were selected from the ORFs having the nucleotide sequence represented by SEQ ID NO: 4958.

The probes having the nucleotide sequence represented by SEQ ID NOS: 61057, 61058, 61059, 61060, 61061, 61062, 61063, 61064, 61065, 61066, 61067 were selected from the ORFs having the nucleotide sequence represented by SEQ ID NO: 4959.

The probes having the nucleotide sequence represented by SEQ ID NOS: 61068, 61069, 61070, 61071, 61072, 61073, 61074, 61075, 61076, 61077, 61078 were selected from the ORFs having the nucleotide sequence represented by SEQ ID NO: 4960.

The probes having the nucleotide sequence represented by SEQ ID NOS: 61079, 61080, 61081, 61082, 61083, 61084, 61085, 61086, 61087, 61088, 61089 were selected from the ORFs having the nucleotide sequence represented by SEQ ID NO: 4961.

The probes having the nucleotide sequence represented by SEQ ID NOS: 61090, 61091, 61092, 61093, 61094, 61095, 61096, 61097, 61098, 61099, 61100 were selected from the ORFs having the nucleotide sequence represented by SEQ ID NO: 4962.

The probes having the nucleotide sequence represented by SEQ ID NOS: 61101, 61102, 61103, 61104, 61105, 61106, 61107, 61108, 61109, 61110, 61111 were selected from the ORFs having the nucleotide sequence represented by SEQ ID NO: 4963.

The probes having the nucleotide sequence represented by SEQ ID NOS: 61112, 61113, 61114, 61115, 61116, 61117, 61118, 61119, 61120, 61121, 61122 were selected from the ORFs having the nucleotide sequence represented by SEQ ID NO: 4964.

The probes having the nucleotide sequence represented by SEQ ID NOS: 61123, 61124, 61125, 61126, 61127, 61128,

61129, 61130, 61131, 61132, 61133 were selected from the ORFs having the nucleotide sequence represented by SEQ ID NO: 4965.

The probes having the nucleotide sequence represented by SEQ ID NOS: 61134, 61135, 61136, 61137, 61138, 61139, 61140, 61141, 61142, 61143, 61144 were selected from the ORFs having the nucleotide sequence represented by SEQ ID NO: 4966.

The probes having the nucleotide sequence represented by SEQ ID NOS: 61145, 61146, 61147, 61148, 61149, 61150, 61151, 61152, 61153, 61154, 61155 were selected from the ORFs having the nucleotide sequence represented by SEQ ID NO: 4967.

The probes having the nucleotide sequence represented by SEQ ID NOS: 61156, 61157, 61158, 61159, 61160, 61161, 61162, 61163, 61164, 61165, 61166 were selected from the ORFs having the nucleotide sequence represented by SEQ ID NO: 4968.

The probes having the nucleotide sequence represented by SEQ ID NOS: 61167, 61168, 61169, 61170, 61171, 61172, 61173, 61174, 61175, 61176, 61177, 61178, 61179, 61180, 61181, 61182, 61183, 61184, 61185, 61186, 61187, 61188 were selected from the ORFs having the nucleotide sequence represented by SEQ ID NO: 4969.

The probes having the nucleotide sequence represented by SEQ ID NOS: 61189, 61190, 61191, 61192, 61193, 61194, 61195, 61196, 61197, 61198, 61199 were selected from the ORFs having the nucleotide sequence represented by SEQ ID NO: 4970.

The probes having the nucleotide sequence represented by SEQ ID NOS: 61200, 61201, 61202, 61203, 61204, 61205, 61206, 61207, 61208, 61209, 61210 were selected from the ORFs having the nucleotide sequence represented by SEQ ID NO: 4971.

The probes having the nucleotide sequence represented by SEQ ID NOS: 61211, 61212, 61213, 61214, 61215, 61216, 61217, 61218, 61219, 61220, 61221 were selected from the ORFs having the nucleotide sequence represented by SEQ ID NO: 4972.

The probes having the nucleotide sequence represented by SEQ ID NOS: 61222, 61223, 61224, 61225, 61226, 61227, 61228, 61229, 61230, 61231, 61232 were selected from the ORFs having the nucleotide sequence represented by SEQ ID NO: 4973.

The probes having the nucleotide sequence represented by SEQ ID NOS: 61233, 61234, 61235, 61236, 61237, 61238, 61239, 61240, 61241, 61242, 61243 were selected from the ORFs having the nucleotide sequence represented by SEQ ID NO: 4974.

The probes having the nucleotide sequence represented by SEQ ID NOS: 61244, 61245, 61246, 61247, 61248, 61249, 61250, 61251, 61252, 61253, 61254 were selected from the ORFs having the nucleotide sequence represented by SEQ ID NO: 4975.

The probes having the nucleotide sequence represented by SEQ ID NOS: 61255, 61256, 61257, 61258, 61259, 61260, 61261, 61262, 61263, 61264, 61265 were selected from the ORFs having the nucleotide sequence represented by SEQ ID NO: 4976.

The probes having the nucleotide sequence represented by SEQ ID NOS: 61266, 61267, 61268, 61269, 61270, 61271, 61272, 61273, 61274, 61275, 61276 were selected from the ORFs having the nucleotide sequence represented by SEQ ID NO: 4977.

The probes having the nucleotide sequence represented by SEQ ID NOS: 61277, 61278, 61279, 61280, 61281, 61282, 61283, 61284, 61285, 61286, 61287 were selected from the ORFs having the nucleotide sequence represented by SEQ ID NO: 4978.

The probes having the nucleotide sequence represented by SEQ ID NOS: 61288, 61289, 61290, 61291, 61292, 61293, 61294, 61295, 61296, 61297, 61298 were selected from the ORFs having the nucleotide sequence represented by SEQ ID NO: 4979.

The probes having the nucleotide sequence represented by SEQ ID NOS: 61299, 61300, 61301, 61302, 61303, 61304, 61305, 61306, 61307, 61308, 61309 were selected from the ORFs having the nucleotide sequence represented by SEQ ID NO: 4980.

The probes having the nucleotide sequence represented by SEQ ID NOS: 61310, 61311, 61312, 61313, 61314, 61315, 61316, 61317, 61318, 61319, 61320 were selected from the ORFs having the nucleotide sequence represented by SEQ ID NO: 4981.

The probes having the nucleotide sequence represented by SEQ ID NOS: 61321, 61322, 61323, 61324, 61325, 61326, 61327, 61328, 61329, 61330, 61331 were selected from the ORFs having the nucleotide sequence represented by SEQ ID NO: 4982.

The probes having the nucleotide sequence represented by SEQ ID NOS: 61332, 61333, 61334, 61335, 61336, 61337, 61338, 61339, 61340, 61341, 61342 were selected from the ORFs having the nucleotide sequence represented by SEQ ID NO: 4983.

The probes having the nucleotide sequence represented by SEQ ID NOS: 61343, 61344, 61345, 61346, 61347, 61348, 61349, 61350, 61351, 61352, 61353 were selected from the ORFs having the nucleotide sequence represented by SEQ ID NO: 4984.

The probes having the nucleotide sequence represented by SEQ ID NOS: 61354, 61355, 61356, 61357, 61358, 61359, 61360, 61361, 61362, 61363, 61364 were selected from the ORFs having the nucleotide sequence represented by SEQ ID NO: 4985.

The probes having the nucleotide sequence represented by SEQ ID NOS: 61365, 61366, 61367, 61368, 61369, 61370, 61371, 61372, 61373, 61374, 61375 were selected from the ORFs having the nucleotide sequence represented by SEQ ID NO: 4986.

The probes having the nucleotide sequence represented by SEQ ID NOS: 61376, 61377, 61378, 61379, 61380, 61381, 61382, 61383, 61384, 61385, 61386 were selected from the ORFs having the nucleotide sequence represented by SEQ ID NO: 4987.

The probes having the nucleotide sequence represented by SEQ ID NOS: 61387, 61388, 61389, 61390, 61391, 61392, 61393, 61394, 61395, 61396, 61397 were selected from the ORFs having the nucleotide sequence represented by SEQ ID NO: 4988.

The probes having the nucleotide sequence represented by. SEQ ID NOS: 61398, 61399, 61400, 61401, 61402, 61403, 61404, 61405, 61406, 61407, 61408 were selected from the ORFs having the nucleotide sequence represented by SEQ ID NO: 4989.

The probes having the nucleotide sequence represented by SEQ ID NOS: 61409, 61410, 61411, 61412, 61413, 61414, 61415, 61416, 61417, 61418, 61419 were selected from the ORFs having the nucleotide sequence represented by SEQ ID NO: 4990.

The probes having the nucleotide sequence represented by SEQ ID NOS: 61420, 61421, 61422, 61423, 61424, 61425, 61426, 61427, 61428, 61429, 61430 were selected from the ORFs having the nucleotide sequence represented by SEQ ID NO: 4991.

The probes having the nucleotide sequence represented by SEQ ID NOS: 61431, 61432, 61433, 61434, 61435, 61436, 61437, 61438, 61439, 61440, 61441 were selected from the ORFs having the nucleotide sequence represented by SEQ ID NO: 4992.

The probes having the nucleotide sequence represented by SEQ ID NOS: 61442, 61443, 61444, 61445, 61446, 61447, 61448, 61449, 61450, 61451, 61452 were selected from the ORFs having the nucleotide sequence represented by SEQ ID NO: 4993.

The probes having the nucleotide sequence represented by SEQ ID NOS: 61453, 61454, 61455, 61456, 61457, 61458, 61459, 61460, 61461, 61462, 61463 were selected from the ORFs having the nucleotide sequence represented by SEQ ID NO: 4994.

The probes having the nucleotide sequence represented by SEQ ID NOS: 61464, 61465, 61466, 61467, 61468, 61469, 61470, 61471, 61472, 61473, 61474 were selected from the ORFs having the nucleotide sequence represented by SEQ ID NO: 4995.

The probes having the nucleotide sequence represented by SEQ ID NOS: 61475, 61476, 61477, 61478, 61479, 61480, 61481, 61482, 61483, 61484, 61485 were selected from the ORFs having the nucleotide sequence represented by SEQ ID NO: 4996.

The probes having the nucleotide sequence represented by SEQ ID NOS: 61486, 61487, 61488, 61489, 61490, 61491, 61492, 61493, 61494, 61495, 61496 were selected from the ORFs having the nucleotide sequence represented by SEQ ID NO: 4997.

The probes having the nucleotide sequence represented by SEQ ID NOS: 61497, 61498, 61499, 61500, 61501, 61502, 61503, 61504, 61505, 61506, 61507 were selected from the ORFs having the nucleotide sequence represented by SEQ ID NO: 4998.

The probes having the nucleotide sequence represented by SEQ ID NOS: 61508, 61509, 61510, 61511, 61512, 61513, 61514, 61515, 61516, 61517, 61518 were selected from the ORFs having the nucleotide sequence represented by SEQ ID NO: 4999.

The probes-having the nucleotide sequence represented by SEQ ID NOS: 61519, 61520, 61521, 61522, 61523, 61524, 61525, 61526, 61527, 61528, 61529 were selected from the ORFs having the nucleotide sequence represented by SEQ ID NO: 5000.

The probes having the nucleotide sequence represented by SEQ ID NOS: 61530, 61531, 61532, 61533, 61534, 61535, 61536, 61537, 61538, 61539, 61540 were selected from the ORFs having the nucleotide sequence represented by SEQ ID NO: 5001.

The probes having-the nucleotide sequence represented by SEQ ID NOS: 61541, 61542, 61543, 61544, 61545, 61546, 61547, 61548, 61549, 61550, 61551 were selected from the ORFs having the nucleotide sequence represented by SEQ ID NO: 5002.

The probes having the nucleotide sequence represented by SEQ ID NOS:. 61552, 61553, 61554, 61555, 61556, 61557, 61558, 61559, 61560, 61561, 61562 were selected from the ORFs having the nucleotide sequence represented by SEQ ID NO: 5003.

The probes having the nucleotide sequence represented by SEQ ID NOS: 61563, 61564, 61565, 61566, 61567, 61568, 61569, 61570, 61571, 61572, 61573 were selected from the ORFs having the nucleotide sequence represented by SEQ ID NO: 5004.

The probes having the nucleotide sequence represented by SEQ ID NOS: 61574, 61575, 61576, 61577, 61578, 61579, 61580, 61581, 61582, 61583, 61584 were selected from the ORFs having the nucleotide sequence represented by SEQ ID NO: 5005.

The probes having the nucleotide sequence represented by SEQ ID NOS: 61585, 61586, 61587, 61588, 61589, 61590, 61591, 61592, 61593, 61594, 61595 were selected from the ORFs having the nucleotide sequence represented by SEQ ID NO: 5006.

The probes having the nucleotide sequence represented by SEQ ID NOS: 61596, 61597, 61598, 61599, 61600, 61601, 61602, 61603, 61604, 61605, 61606 were selected from the ORFs having the nucleotide sequence represented by SEQ ID NO: 5007.

The probes having the nucleotide sequence represented by SEQ ID NOS: 61607, 61608, 61609, 61610, 61611, 61612, 61613, 61614, 61615, 61616, 61617 were selected from the ORFs having the nucleotide sequence represented by SEQ ID NO: 5008.

The probes having the nucleotide sequence represented by SEQ ID NOS: 61618, 61619, 61620, 61621, 61622, 61623, 61624, 61625, 61626, 61627, 61628 were selected from the ORFs having the nucleotide sequence represented by SEQ ID NO: 5009.

The probes having the nucleotide sequence represented by SEQ ID NOS: 61629, 61630, 61631, 61632, 61633, 61634, 61635, 61636, 61637, 61638, 61639 were selected from the ORFs having the nucleotide sequence represented by SEQ ID NO: 5010.

The probes having the nucleotide sequence represented by SEQ ID NOS: 61640, 61641, 61642, 61643, 61644, 61645, 61646, 61647, 61648, 61649, 61650 were selected from the ORFs having the nucleotide sequence represented by SEQ ID NO: 5011.

The probes having the nucleotide sequence represented by SEQ ID NOS: 61651, 61652, 61653, 61654, 61655, 61656, 61657, 61658, 61659, 61660, 61661 were selected from the ORFs having the nucleotide sequence represented by SEQ ID NO: 5012.

The probes having the nucleotide sequence represented by SEQ ID NOS: 61662, 61663, 61664, 61665, 61666, 61667, 61668, 61669, 61670, 61671, 61672 were selected from the ORFs having the nucleotide sequence represented by SEQ ID NO: 5013.

The probes having the nucleotide sequence represented by SEQ ID NOS: 61673, 61674, 61675, 61676, 61677, 61678, 61679, 61680, 61681, 61682, 61683 were selected from the ORFs having the nucleotide sequence represented by SEQ ID NO: 5014.

The probes having the nucleotide sequence represented by SEQ ID NOS: 61684, 61685, 61686, 61687, 61688, 61689, 61690, 61691, 61692, 61693, 61694 were selected from the ORFs having the nucleotide sequence represented by SEQ ID NO: 5015.

The probes having the nucleotide sequence represented by SEQ ID NOS: 61695, 61696, 61697, 61698, 61699, 61700, 61701, 61702, 61703, 61704, 61705 were selected from the ORFs having the nucleotide sequence represented by SEQ ID NO: 5016.

The probes having the nucleotide sequence represented by SEQ ID NOS: 61706, 61707, 61708, 61709, 61710, 61711, 61712, 61713, 61714, 61715, 61716 were selected from the ORFs having the nucleotide sequence represented by SEQ ID NO: 5017.

The probes having the nucleotide sequence represented by SEQ ID NOS: 61717, 61718, 61719, 61720, 61721, 61722, 61723, 61724, 61725, 61726, 61727 were selected from the ORFs having the nucleotide sequence represented by SEQ ID NO: 5018.

The probes having the nucleotide sequence represented by SEQ ID NOS: 61728, 61729, 61730, 61731, 61732, 61733, 61734, 61735, 61736, 6.1737, 61738 were selected from the ORFs having the nucleotide sequence represented by SEQ ID NO: 5019.

The probes having the nucleotide sequence represented by SEQ ID NOS: 61739, 61740, 61741, 61742, 61743, 61744, 61745, 61746, 61747, 61748, 61749 were selected from the ORFs having the nucleotide sequence represented by SEQ ID NO: 5020.

The probes having the nucleotide sequence represented by SEQ ID NOS: 61750, 61751, 61752, 61753, 61754, 61755, 61756, 61757, 61758, 61759, 61760 were selected from the ORFs having the nucleotide sequence represented by SEQ ID NO: 5021.

The probes having the nucleotide sequence represented by SEQ ID NOS: 61761, 61762, 61763, 61764, 61765, 61766, 61767, 61768, 61769, 61770, 61771 were selected from the ORFs having the nucleotide sequence represented by SEQ ID NO: 5022.

The probes having the nucleotide sequence represented by SEQ ID NOS: 61772, 61773, 61774, 61775, 61776, 61777, 61778, 61779, 61780, 61781, 61782 were selected from the ORFs having the-nucleotide sequence represented by SEQ ID NO: 5023.

The probes having the nucleotide sequence represented by SEQ ID NOS: 61783, 61784, 61785, 61786, 61787, 61788, 61789, 61790, 61791, 61792, 61793 were selected from the ORFs having the nucleotide sequence represented by SEQ ID NO: 5024.

The probes having the nucleotide sequence represented by SEQ ID NOS: 61794, 61795, 61796, 61797, 61798, 61799, 61800, 61801, 61802, 61803, 61804 were selected from the ORFs having the nucleotide sequence represented by SEQ ID NO: 5025.

The probes having the nucleotide sequence represented by SEQ ID NOS: 61805, 61806, 61807, 61808, 61809, 61810, 61811, 61812, 61813, 61814, 61815 were selected from the ORFs having the nucleotide sequence represented by SEQ ID NO: 5026.

The probes having the nucleotide sequence represented by SEQ ID NOS: 61816, 61817, 61818, 61819, 61820, 61821, 61822, 61823, 61824, 61825, 61826 were selected from the ORFs having the nucleotide sequence represented by SEQ ID NO: 5027.

The probes having the nucleotide sequence represented by SEQ ID NOS: 61827, 61828, 61829, 61830, 61831, 61832, 61833, 61834, 61835, 61836, 61837 were selected from the ORFs having the nucleotide sequence represented by SEQ ID NO: 5028.

The probes having the nucleotide sequence represented by SEQ ID NOS: 61838, 61839, 61840, 61841, 61842, 61843, 61844, 61845, 61846, 61847, 61848 were selected from the ORFs having the nucleotide sequence represented by SEQ ID NO: 5029.

The probes having the nucleotide sequence represented by SEQ ID NOS: 61849, 61850, 61851, 61852, 61853, 61854, 61855, 61856, 61857, 61858, 61859 were selected from the ORFs having the nucleotide sequence represented by SEQ ID NO: 5030.

The probes having the nucleotide sequence represented by SEQ ID NOS: 61860, 61861, 61862, 61863, 61864, 61865, 61866, 61867, 61868, 61869, 61870 were selected from the ORFs having the nucleotide sequence represented by SEQ ID NO: 5031.

The probes having the nucleotide sequence represented by SEQ ID NOS: 61871, 61872, 61873, 61874, 61875, 61876, 61877, 61878, 61879, 61880, 61881 were selected from the ORFs having the nucleotide sequence represented by SEQ ID NO: 5032.

The probes having the nucleotide sequence represented by SEQ ID NOS: 61882, 61883, 61884, 61885, 61886, 61887, 61888, 61889, 61890, 61891, 61892 were selected from the ORFs having the nucleotide sequence represented by SEQ ID NO: 5033.

The probes having the nucleotide sequence represented by SEQ ID NOS: 61893, 61894, 61895, 61896, 61897, 61898, 61899, 61900, 61901, 61902, 61903 were selected from the ORFs having the nucleotide sequence represented by SEQ ID NO: 5034.

The probes having the nucleotide sequence represented by SEQ ID NOS: 61904, 61905, 61906, 61907, 61908, 61909, 61910, 61911, 61912, 61913, 61914 were selected from the ORFs having the nucleotide sequence represented by SEQ ID NO: 5035.

The probes having the nucleotide sequence represented by SEQ ID NOS: 61915, 61916, 61917, 61918, 61919, 61920, 61921, 61922, 61923, 61924, 61925 were selected from the ORFs having the nucleotide sequence represented by SEQ ID NO: 5036.

The probes having the nucleotide sequence represented by SEQ ID NOS: 61926, 61927, 61928, 61929, 61930, 61931, 61932, 61933, 61934, 61935, 61936 were selected from the ORFs having the nucleotide sequence represented by SEQ ID NO: 5037.

The probes having the nucleotide sequence represented by SEQ ID NOS: 61937, 61938, 61939, 61940, 61941, 61942, 61943, 61944, 61945, 61946, 61947 were selected from the ORFs having the nucleotide sequence represented by SEQ ID NO: 5038.

The probes having the nucleotide sequence represented by SEQ ID NOS: 61948, 61949, 61950, 61951, 61952, 61953, 61954, 61955, 61956, 61957, 61958 were selected from the ORFs having the nucleotide sequence represented by SEQ ID NO: 5039.

The probes having the nucleotide sequence represented by SEQ ID NOS: 61959, 61960, 61961, 61962, 61963, 61964, 61965, 61966, 61967, 61968, 61969 were selected from the ORFs having the nucleotide sequence represented by SEQ ID NO:. 5040.

The probes having the nucleotide sequence represented by SEQ ID NOS: 61970, 61971, 61972, 61973, 61974, 61975, 61976, 61977, 61978, 61979, 61980, 61981, 61982, 61983, 61984, 61985, 61986, 61987, 61988, 61989, 61990, 61991 were selected from the ORFs having the nucleotide sequence represented by SEQ ID NO: 5041.

The probes having the nucleotide sequence represented by SEQ ID NOS: 61992, 61993, 61994, 61995, 61996, 61997, 61998, 61999, 62000, 62001, 62002 were selected from the ORFs having the nucleotide sequence represented by SEQ ID NO: 5042.

The probes having the nucleotide sequence represented by SEQ ID NOS: 62003, 62004, 62005, 62006, 62007, 62008, 62009, 62010, 62011, 62012, 62013 were selected from the ORFs having the nucleotide sequence represented by SEQ ID NO: 5043.

The probes having the nucleotide sequence represented by SEQ ID NOS: 62014, 62015, 62016, 62017, 62018, 62019, 62020, 62021, 62022, 62023, 62024 were selected from the ORFs having the nucleotide sequence represented by SEQ ID NO: 5044.

The probes having the nucleotide sequence represented by SEQ ID NOS: 62025, 62026, 62027, 62028, 62029, 62030, 62031, 62032, 62033, 62034, 62035 were selected from the ORFs having the nucleotide sequence represented by SEQ ID NO: 5045.

The probes having the nucleotide sequence represented by SEQ ID NOS: 62036, 62037, 62038, 62039, 62040, 62041, 62042, 62043, 62044, 62045, 62046 were selected from the ORFs having the nucleotide sequence represented by SEQ ID NO: 5046.

The probes having the nucleotide sequence represented by SEQ ID NOS: 62047, 62048, 62049, 62050, 62051, 62052, 62053, 62054, 62055, 62056, 62057 were selected from the ORFs having the nucleotide sequence represented by SEQ ID NO: 5047.

The probes having the nucleotide sequence represented by SEQ ID NOS: 62058, 62059, 62060, 62061, 62062, 62063, 62064, 62065, 62066, 62067, 62068 were selected from the ORFs having the nucleotide sequence represented by SEQ ID NO: 5048.

The probes having the nucleotide sequence represented by SEQ ID NOS: 62069, 62070, 62071, 62072, 62073, 62074, 62075, 62076, 62077, 62078, 62079 were selected from the ORFs having the nucleotide sequence represented by SEQ ID NO: 5049.

The probes having the nucleotide sequence represented by SEQ ID NOS: 62080, 62081, 62082, 62083, 62084, 62085, 62086, 62087, 62088, 62089, 62090 were selected from the ORFs having the nucleotide sequence represented by SEQ ID NO: 5050.

The probes having the nucleotide sequence represented by SEQ ID NOS: 62091, 62092, 62093, 62094, 62095, 62096, 62097, 62098, 62099, 62100, 62101 were selected from the ORFs having the nucleotide sequence represented by SEQ ID NO: 5051.

The probes having the nucleotide sequence represented by SEQ ID NOS: 62102, 62103, 62104, 62105, 62106, 62107, 62108, 62109, 62110, 62111, 62112 were selected from the ORFs having the nucleotide sequence represented by SEQ ID NO: 5052.

The probes having the nucleotide sequence represented by SEQ ID NOS: 62113, 62114, 62115, 62116, 62117, 62118, 62119, 62120, 62121, 62122, 62123 were selected from the ORFs having the nucleotide sequence represented by SEQ ID NO: 5053.

The probes having the nucleotide sequence represented by SEQ ID NOS: 62124, 62125, 62126, 62127, 62128, 62129, 62130, 62131, 62132, 62133, 62134 were selected from the ORFs having the nucleotide sequence represented by SEQ ID NO: 5054.

The probes having the nucleotide sequence represented by SEQ ID NOS: 62135, 62136, 62137, 62138, 62139, 62140, 62141, 62142, 62143, 62144, 62145 were selected from the ORFs having the nucleotide sequence represented by SEQ ID NO: 5055.

The probes having the nucleotide sequence represented by SEQ ID NOS: 62146, 62147, 62148, 62149, 62150, 62151, 62152, 62153, 62154, 62155, 62156 were selected from the ORFs having the nucleotide sequence represented by SEQ ID NO: 5056.

The probes having the nucleotide sequence represented by SEQ ID NOS: 62157, 62158, 62159, 62160, 62161, 62162, 62163, 62164, 62165, 62166, 62167 were selected from the ORFs having the nucleotide sequence represented by SEQ ID NO: 5057.

The probes having the nucleotide sequence represented by SEQ ID NOS: 62168, 62169, 62170, 62171, 62172, 62173, 62174, 62175, 62176, 62177, 62178 were selected from the ORFs having the nucleotide sequence represented by SEQ ID NO: 5058.

The probes having the nucleotide sequence represented by SEQ ID NOS: 62179, 62180, 62181, 62182, 62183, 62184, 62185, 62186, 62187, 62188, 62189 were selected from the ORFs having the nucleotide sequence represented by SEQ ID NO: 5059.

The probes having the nucleotide sequence represented by SEQ ID NOS: 62190, 62191, 62192, 62193, 62194, 62195, 62196, 62197, 62198, 62199, 62200 were selected from the ORFs having the nucleotide sequence represented by SEQ ID NO: 5060.

The probes having the nucleotide sequence represented by SEQ ID NOS: 62201, 62202, 62203, 62204, 62205, 62206, 62207, 62208, 62209, 62210, 62211 were selected from the ORFs having the nucleotide sequence represented by SEQ ID NO: 5061.

The probes having the nucleotide sequence represented by SEQ ID NOS: 62212, 62213, 62214, 62215, 62216, 62217, 62218, 62219, 62220, 62221, 62222 were selected from the ORFs having the nucleotide sequence represented by SEQ ID NO: 5062.

The probes having the nucleotide sequence represented by SEQ ID NOS: 62223, 62224, 62225, 62226, 62227, 62228, 62229, 62230, 62231, 62232, 62233 were selected from the ORFs having the nucleotide sequence represented by SEQ ID NO: 5063.

The probes having the nucleotide sequence represented by SEQ ID NOS: 62234, 62235, 62236, 62237, 62238, 62239, 62240, 62241, 62242, 62243, 62244 were selected from the ORFs having the nucleotide sequence represented by SEQ ID NO: 5064.

The probes having the nucleotide sequence represented by SEQ ID NOS: 62245, 62246, 62247, 62248, 62249, 62250, 62251, 62252, 62253, 62254, 62255 were selected from the ORFs having the nucleotide sequence represented by SEQ ID NO: 5065.

The probes having the nucleotide sequence represented by SEQ ID NOS: 62256, 62257, 62258, 62259, 62260, 62261, 62262, 62263, 62264, 62265, 62266 were selected from the ORFs having the nucleotide sequence represented by SEQ ID NO: 5066.

The probes having the nucleotide sequence represented by SEQ ID NOS: 62267, 62268, 62269, 62270, 62271, 62272, 62273, 62274, 62275, 62276, 62277 were selected from the ORFs having the nucleotide sequence represented by SEQ ID NO: 5067.

The probes having the nucleotide sequence represented by SEQ ID NOS: 62278, 62279, 62280, 62281, 62282, 62283, 62284, 62285, 62286, 62287, 62288 were selected from the ORFs having the nucleotide sequence represented by SEQ ID NO: 5068.

The probes having the nucleotide sequence represented by SEQ ID NOS: 62289, 62290, 62291, 62292, 62293, 62294, 62295, 62296, 62297, 62298, 62299 were selected from the ORFs having the nucleotide sequence represented by SEQ ID NO: 5069.

The probes having the nucleotide sequence represented by SEQ ID NOS: 62300, 62301, 62302, 62303, 62304, 62305, 62306, 62307, 62308, 62309, 62310 were selected from the ORFs having the nucleotide sequence represented by SEQ ID NO: 5070.

The probes having the nucleotide sequence represented by SEQ ID NOS: 62311, 62312, 62313, 62314, 62315, 62316, 62317, 62318, 62319, 62320, 62321 were selected from the ORFs having the nucleotide sequence represented by SEQ ID NO: 5071.

The probes having the nucleotide sequence represented by SEQ ID NOS: 62322, 62323, 62324, 62325, 62326, 62327, 62328, 62329, 62330, 62331, 62332 were selected from the ORFs having the nucleotide sequence represented by SEQ ID NO: 5072.

The probes having the nucleotide sequence represented by SEQ ID NOS: 62333, 62334, 62335, 62336, 62337, 62338, 62339, 62340, 62341, 62342, 62343 were selected from the ORFs having the nucleotide sequence represented by SEQ ID NO: 5073.

The probes having the nucleotide sequence represented by SEQ ID NOS: 62344, 62345, 62346, 62347, 62348, 62349, 62350, 62351, 62352, 62353, 62354 were selected from the ORFs having the nucleotide sequence represented by SEQ ID NO: 5074.

The probes having the nucleotide sequence represented by SEQ ID NOS: 62355, 62356, 62357, 62358, 62359, 62360, 62361, 62362, 62363, 62364, 62365 were selected from the ORFs having the nucleotide sequence represented by SEQ ID NO: 5075.

The probes having the nucleotide sequence represented by SEQ ID NOS: 62366, 62367, 62368, 62369, 62370, 62371, 62372, 62373, 62374, 62375, 62376 were selected from the ORFs having the nucleotide sequence represented by SEQ ID NO: 5076.

The probes having the nucleotide sequence represented by SEQ ID NOS: 62377, 62378, 62379, 62380, 62381, 62382, 62383, 62384, 62385, 62386, 62387, 62388, 62389, 62390, 62391, 62392, 62393, 62394, 62395, 62396, 62397, 62398 were selected from the ORFs having the nucleotide sequence represented by SEQ ID NO: 5077.

The probes having the nucleotide sequence represented by SEQ ID NOS: 62399, 62400, 62401, 62402, 62403, 62404, 62405, 62406, 62407, 62408, 62409 were selected from the ORFs having the nucleotide sequence represented by SEQ ID NO: 5078.

The probes having the nucleotide sequence represented by SEQ ID NOS: 62410, 62411, 62412, 62413, 62414, 62415, 62416, 62417, 62418, 62419, 62420 were selected from the ORFs having the nucleotide sequence represented by SEQ ID NO: 5079.

The probes having the nucleotide sequence represented by SEQ ID NOS: 62421, 62422, 62423, 62424, 62425, 62426, 62427, 62428, 62429, 62430, 62431 were selected from the ORFs having the nucleotide sequence represented by SEQ ID NO: 5080.

The probes having the nucleotide sequence represented by SEQ ID NOS: 62432, 62433, 62434, 62435, 62436, 62437, 62438, 62439, 62440, 62441, 62442 were selected from the ORFs having the nucleotide sequence represented by SEQ ID NO: 5081.

The probes having the nucleotide sequence represented by SEQ ID NOS: 62443, 62444, 62445, 62446, 62447, 62448, 62449, 62450, 62451, 62452, 62453 were selected from the ORFs having the nucleotide sequence represented by SEQ ID NO: 5082.

The probes having the nucleotide sequence represented by SEQ ID NOS: 62454, 62455, 62456, 62457, 62458, 62459, 62460, 62461, 62462, 62463, 62464 were selected from the ORFs having the nucleotide sequence represented by SEQ ID NO: 5083.

The probes having the nucleotide sequence represented by SEQ ID NOS: 62465, 62466, 62467, 62468, 62469, 62470, 62471, 62472, 62473, 62474, 62475 were selected from the ORFs having the nucleotide sequence represented by SEQ ID NO: 5084.

The probes having the nucleotide sequence represented by SEQ ID NOS: 62476, 62477, 62478, 62479, 62480, 62481, 62482, 62483, 62484, 62485, 62486 were selected from the ORFs having the nucleotide sequence represented by SEQ ID NO: 5085.

The probes having the nucleotide sequence represented by SEQ ID NOS: 62487, 62488, 62489, 62490, 62491, 62492, 62493, 62494, 62495, 62496, 62497 were selected from the ORFs having the nucleotide sequence represented by SEQ ID NO: 5086.

The probes having the nucleotide sequence represented by SEQ ID NOS: 62498, 62499, 62500, 62501, 62502, 62503, 62504, 62505, 62506, 62507, 62508 were selected from the ORFs having the nucleotide sequence represented by SEQ ID NO: 5087.

The probes having the nucleotide sequence represented by SEQ ID NOS: 62509, 62510, 62511, 62512, 62513, 62514, 62515, 62516, 62517, 62518, 62519 were selected from the ORFs having the nucleotide sequence represented by SEQ ID NO: 5088.

The probes having the nucleotide sequence represented by SEQ ID NOS: 62520, 62521, 62522, 62523, 62524, 62525, 62526, 62527, 62528, 62529, 62530 were selected from the ORFs having the nucleotide sequence represented by SEQ ID NO: 5089.

The probes having the nucleotide sequence represented by SEQ ID NOS: 62531, 62532, 62533, 62534, 62535, 62536, 62537, 62538, 62539, 62540, 62541 were selected from the ORFs having the nucleotide sequence represented by SEQ ID NO: 5090.

The probes having the nucleotide sequence represented by SEQ ID NOS: 62542, 62543, 62544, 62545, 62546, 62547, 62548, 62549, 62550, 62551, 62552 were selected from the ORFs having the nucleotide sequence represented by SEQ ID NO: 5091.

The probes having the nucleotide sequence represented by SEQ ID NOS: 62553, 62554, 62555, 62556, 62557, 62558, 62559, 62560, 62561, 62562, 62563 were selected from the ORFs having the nucleotide sequence represented by SEQ ID NO: 5092.

The probes having the nucleotide sequence represented by SEQ ID NOS: 62564, 62565, 62566, 62567, 62568, 62569, 62570, 62571, 62572, 62573, 62574 were selected from the ORFs having the nucleotide sequence represented by SEQ ID NO: 5093.

The probes having the nucleotide sequence represented by SEQ ID NOS: 62575, 62576, 62577, 62578, 62579, 62580, 62581, 62582, 62583, 62584, 62585 were selected from the ORFs having the nucleotide sequence represented by SEQ ID NO: 5094.

The probes having the nucleotide sequence represented by SEQ ID NOS: 62586, 62587, 62588, 62589, 62590, 62591, 62592, 62593, 62594, 62595, 62596 were selected from the ORFs having the nucleotide sequence represented by SEQ ID NO: 5095.

The probes having the nucleotide sequence represented by SEQ ID NOS: 62597, 62598, 62599, 62600, 62601, 62602, 62603, 62604, 62605, 62606, 62607 were selected from the ORFs having the nucleotide sequence represented by SEQ ID NO: 5096.

The probes having the nucleotide sequence represented by SEQ ID NOS: 62608, 62609, 62610, 62611, 62612, 62613, 62614, 62615, 62616, 62617, 62618 were selected from the ORFs having the nucleotide sequence represented by SEQ ID NO: 5097.

The probes having the nucleotide sequence represented by SEQ ID NOS: 62619, 62620, 62621, 62622, 62623, 62624, 62625, 62626, 62627, 62628, 62629 were selected from the ORFs having the nucleotide sequence represented by SEQ ID NO: 5098.

The probes having the nucleotide sequence represented by SEQ ID NOS: 62630, 62631, 62632, 62633, 62634, 62635, 62636, 62637, 62638, 62639, 62640 were selected from the ORFs having the nucleotide sequence represented by SEQ ID NO: 5099.

The probes having the nucleotide sequence represented by SEQ ID NOS: 62641, 62642, 62643, 62644, 62645, 62646, 62647, 62648, 62649, 62650, 62651 were selected from the ORFs having the nucleotide sequence represented by SEQ ID NO: 5100.

The probes having the nucleotide sequence represented by SEQ ID NOS: 62652, 62653, 62654, 62655, 62656, 62657, 62658, 62659, 62660, 62661, 62662 were selected from the ORFs having the nucleotide sequence represented by SEQ ID NO: 5101.

The probes having the nucleotide sequence represented by SEQ ID NOS: 62663, 62664, 62665, 62666, 62667, 62668, 62669, 62670, 62671, 62672, 62673 were selected from the ORFs having the nucleotide sequence represented by SEQ ID NO: 5102.

The probes having the nucleotide sequence represented by SEQ ID NOS: 62674, 62675, 62676, 62677, 62678, 62679, 62680, 62681, 62682, 62683, 62684 were selected from the ORFs having the nucleotide sequence represented by SEQ ID NO: 5103.

The probes having the nucleotide sequence represented by SEQ ID NOS: 62685, 62686, 62687, 62688, 62689, 62690, 62691, 62692, 62693, 62694, 62695 were selected from the ORFs having the nucleotide sequence represented by SEQ ID NO: 5104.

The probes having the nucleotide sequence represented by SEQ ID NOS: 62696, 62697, 62698, 62699, 62700, 62701, 62702, 62703, 62704, 62705, 62706 were selected from the ORFs having the nucleotide sequence represented by SEQ ID NO: 5105.

The probes having the nucleotide sequence represented by SEQ ID NOS: 62707, 62708, 62709, 62710, 62711, 62712, 62713, 62714, 62715, 62716, 62717 were selected from the ORFs having the nucleotide sequence represented by SEQ ID NO: 5106.

The probes having the nucleotide sequence represented by SEQ ID NOS: 62718, 62719, 62720, 62721, 62722, 62723, 62724, 62725, 62726, 62727, 62728 were selected from the ORFs having the nucleotide sequence represented by SEQ ID NO: 5107.

The probes having the nucleotide sequence represented by SEQ ID NOS: 62729, 62730, 62731, 62732, 62733, 62734, 62735, 62736, 62737, 62738, 62739 were selected from the ORFs having the nucleotide sequence represented by SEQ ID NO: 5108.

The probes having the nucleotide sequence represented by SEQ ID NOS: 62740, 62741, 62742, 62743, 62744, 62745, 62746, 62747, 62748, 62749, 62750 were selected from the ORFs having the nucleotide sequence represented by SEQ ID NO: 5109.

The probes having the nucleotide sequence represented by SEQ ID NOS: 62751, 62752, 62753, 62754, 62755, 62756, 62757, 62758, 62759, 62760, 62761 were selected from the ORFs having the nucleotide sequence represented by SEQ ID NO: 5110.

The probes having the nucleotide sequence represented by SEQ ID NOS: 62762, 62763, 62764, 62765, 62766, 62767, 62768, 62769, 62770, 62771, 62772 were selected from the ORFs having the nucleotide sequence represented by SEQ ID NO: 5111.

The probes having the nucleotide sequence represented by SEQ ID NOS: 62773, 62774, 62775, 62776, 62777, 62778, 62779, 62780, 62781, 62782, 62783 were selected from the ORFs having the nucleotide sequence represented by SEQ ID NO: 5112.

The probes having the nucleotide sequence represented by SEQ ID NOS: 62784, 62785, 62786, 62787, 62788, 62789, 62790, 62791, 62792, 62793, 62794 were selected from the ORFs having the nucleotide sequence represented by SEQ ID NO: 5113.

The probes having the nucleotide sequence represented by SEQ ID NOS: 62795, 62796, 62797, 62798, 62799, 62800, 62801, 62802, 62803, 62804, 62805 were selected from the ORFs having the nucleotide sequence represented by SEQ ID NO: 5114.

The probes having the nucleotide sequence represented by SEQ ID NOS: 62806, 62807, 62808, 62809, 62810, 62811, 62812, 62813, 62814, 62815, 62816 were selected from the ORFs having the nucleotide sequence represented by SEQ ID NO: 5115.

The probes having the nucleotide sequence represented by SEQ ID NOS: 62817, 62818, 62819, 62820, 62821, 62822, 62823, 62824, 62825, 62826, 62827 were selected from the ORFs having the nucleotide sequence represented by SEQ ID NO: 5116.

The probes having the nucleotide sequence represented by SEQ ID NOS: 62828, 62829, 62830, 62831, 62832, 62833, 62834, 62835, 62836, 62837, 62838 were selected from the ORFs having the nucleotide sequence represented by SEQ ID NO: 5117.

The probes having the nucleotide sequence represented by SEQ ID NOS: 62839, 62840, 62841, 62842, 62843, 62844, 62845, 62846, 62847, 62848, 62849 were selected from the ORFs having the nucleotide sequence represented by SEQ ID NO: 5118.

The probes having the nucleotide sequence represented by SEQ ID NOS: 62850, 62851, 62852, 62853, 62854, 62855, 62856, 62857, 62858, 62859, 62860 were selected from the ORFs having the nucleotide sequence represented by SEQ ID NO: 5119.

The probes having the nucleotide sequence represented by SEQ ID NOS: 62861, 62862, 62863, 62864, 62865, 62866, 62867, 62868, 62869, 62870, 62871 were selected from the ORFs having the nucleotide sequence represented by SEQ ID NO: 5120.

The probes having the nucleotide sequence represented by SEQ ID NOS: 62872, 62873, 62874, 62875, 62876, 62877, 62878, 62879, 62880, 62881, 62882 were selected from the ORFs having the nucleotide sequence represented by SEQ ID NO: 5121.

The probes having the nucleotide sequence represented by SEQ ID NOS: 62883, 62884, 62885, 62886, 62887, 62888, 62889, 62890, 62891, 62892, 62893 were selected from the ORFs having the nucleotide sequence represented by SEQ ID NO: 5122.

The probes having the nucleotide sequence represented by SEQ ID NOS: 62894, 62895, 62896, 62897, 62898, 62899, 62900, 62901, 62902, 62903, 62904 were selected from the ORFs having the nucleotide sequence represented by SEQ ID NO: 5123.

The probes having the nucleotide sequence represented by SEQ ID NOS: 62905, 62906, 62907, 62908, 62909, 62910, 62911, 62912, 62913, 62914, 62915 were selected from the ORFs having the nucleotide sequence represented by SEQ ID NO: 5124.

The probes having the nucleotide sequence represented by SEQ ID NOS: 62916, 62917, 62918, 62919, 62920, 62921, 62922, 62923, 62924, 62925, 62926 were selected from the ORFs having the nucleotide sequence represented by SEQ ID NO: 5125.

The probes having the nucleotide sequence represented by SEQ ID NOS: 62927, 62928, 62929, 62930, 62931, 62932, 62933, 62934, 62935, 62936, 62937 were selected from the ORFs having the nucleotide sequence represented by SEQ ID NO: 5126.

The probes having the nucleotide sequence represented by SEQ ID NOS: 62938, 62939, 62940, 62941, 62942, 62943, 62944, 62945, 62946, 62947, 62948 were selected from the ORFs having the nucleotide sequence represented by SEQ ID NO: 5127.

The probes having the nucleotide sequence represented by SEQ ID NOS: 62949, 62950, 62951, 62952, 62953, 62954, 62955, 62956, 62957, 62958, 62959 were selected from the ORFs having the nucleotide sequence represented by SEQ ID NO: 5128.

The probes having the nucleotide sequence represented by SEQ ID NOS: 62960, 62961, 62962, 62963, 62964, 62965, 62966, 62967, 62968, 62969, 62970 were selected from the ORFs having the nucleotide sequence represented by SEQ ID NO: 5129.

The probes having the nucleotide sequence represented by SEQ ID NOS: 62971, 62972, 62973, 62974, 62975, 62976, 62977, 62978, 62979, 62980, 62981 were selected from the ORFs having the nucleotide sequence represented by SEQ ID NO: 5130.

The probes having the nucleotide sequence represented by SEQ ID NOS: 62982, 62983, 62984, 62985, 62986, 62987, 62988, 62989, 62990, 62991, 62992 were selected from the ORFs having the nucleotide sequence represented by SEQ ID NO: 5131.

The probes having the nucleotide sequence represented by SEQ ID NOS: 62993, 62994, 62995, 62996, 62997, 62998, 62999, 63000, 63001, 63002, 63003 were selected from the ORFs having the nucleotide sequence represented by SEQ ID NO: 5132.

The probes having the nucleotide sequence represented by SEQ ID NOS: 63004, 63005, 63006, 63007, 63008, 63009, 63010, 63011, 63012, 63013, 63014 were selected from the ORFs having the nucleotide sequence represented by SEQ ID NO: 5133.

The probes having the nucleotide sequence represented by SEQ ID NOS: 63015, 63016, 63017, 63018, 63019, 63020, 63021, 63022, 63023, 63024, 63025, 63026, 63027, 63028, 63029, 63030, 63031, 63032, 63033, 63034, 63035, 63036 were selected from the ORFs having the nucleotide sequence represented by SEQ ID NO: 5134.

The probes having the nucleotide sequence represented by SEQ ID NOS: 63037, 63038, 63039, 63040, 63041, 63042, 63043, 63044, 63045, 63046, 63047 were selected from the ORFs having the nucleotide sequence represented by SEQ ID NO: 5135.

The probes having the nucleotide sequence represented by SEQ ID NOS: 63048, 63049, 63050, 63051, 63052, 63053, 63054, 63055, 63056, 63057, 63058 were selected from the ORFs having the nucleotide sequence represented by SEQ ID NO: 5136.

The probes having the nucleotide sequence represented by SEQ ID NOS: 63059, 63060, 63061, 63062, 63063, 63064, 63065, 63066, 63067, 63068, 63069 were selected from the ORFs having the nucleotide sequence represented by SEQ ID NO: 5137.

The probes having the nucleotide sequence represented by SEQ ID NOS: 63070, 63071, 63072, 63073, 63074, 63075, 63076, 63077, 63078, 63079, 63080 were selected from the ORFs having the nucleotide sequence represented by SEQ ID NO: 5138.

The probes having the nucleotide sequence represented by SEQ ID NOS: 63081, 63082, 63083, 63084, 63085, 63086, 63087, 63088, 63089, 63090, 63091 were selected from the ORFs having the nucleotide sequence represented by SEQ ID NO: 5139.

The probes having the nucleotide sequence represented by SEQ ID NOS: 63092, 63093, 63094, 63095, 63096, 63097, 63098, 63099, 63100 were selected from the ORFs having the nucleotide sequence represented by SEQ ID NO: 5140.

The probes having the nucleotide sequence represented by SEQ ID NOS: 63101, 63102, 63103, 63104, 63105, 63106, 63107, 63108, 63109, 63110, 63111 were selected from the ORFs having the nucleotide sequence represented by SEQ ID NO: 5141.

The probes having the nucleotide sequence represented by SEQ ID NOS: 63112, 63113, 63114, 63115, 63116, 63117, 63118, 63119, 63120, 63121, 63122 were selected from the ORFs having the nucleotide sequence represented by SEQ ID NO: 5142.

The probes having the nucleotide sequence represented by SEQ ID NOS: 63123, 63124, 63125, 63126, 63127, 63128, 63129, 63130, 63131, 63132, 63133 were selected from the ORFs having the nucleotide sequence represented by SEQ ID NO: 5143.

The probes having the nucleotide sequence represented by SEQ ID NOS: 63134, 63135, 63136, 63137, 63138, 63139, 63140, 63141, 63142, 63143, 63144 were selected from the ORFs having the nucleotide sequence represented by SEQ ID NO: 5144.

The probes having the nucleotide sequence represented by SEQ ID NOS: 63145, 63146, 63147, 63148, 63149, 63150, 63151, 63152, 63153, 63154, 63155 were selected from the ORFs having the nucleotide sequence represented by SEQ ID NO: 5145.

The probes having the nucleotide sequence represented by SEQ ID NOS: 63156, 63157, 63158, 63159, 63160, 63161, 63162, 63163, 63164, 63165, 63166 were selected from the ORFs having the nucleotide sequence represented by SEQ ID NO: 5146.

The probes having the nucleotide sequence represented by SEQ ID NOS: 63167, 63168, 63169, 63170, 63171, 63172, 63173, 63174, 63175, 63176, 63177 were selected from the ORFs having the nucleotide sequence represented by SEQ ID NO: 5147.

The probes having the nucleotide sequence represented by SEQ ID NOS: 63178, 63179, 63180, 63181, 63182, 63183, 63184, 63185, 63186, 63187, 63188 were selected from the ORFs having the nucleotide sequence represented by SEQ ID NO: 5148.

The probes having the nucleotide sequence represented by SEQ ID NOS: 63189, 63190, 63191, 63192, 63193, 63194, 63195, 63196, 63197, 63198, 63199 were selected from the ORFs having the nucleotide sequence represented by SEQ ID NO: 5149.

The probes having the nucleotide sequence represented by SEQ ID NOS: 63200, 63201, 63202, 63203, 63204, 63205, 63206, 63207, 63208, 63209, 63210 were selected from the ORFs having the nucleotide sequence represented by SEQ ID NO: 5150.

The probes having the nucleotide sequence represented by SEQ ID NOS: 63211, 63212, 63213, 63214, 63215, 63216, 63217, 63218, 63219, 63220, 63221 were selected from the ORFs having the nucleotide sequence represented by SEQ ID NO: 5151.

The probes having the nucleotide sequence represented by SEQ ID NOS: 63222, 63223, 63224, 63225, 63226, 63227, 63228, 63229, 63230, 63231, 63232 were selected from the ORFs having the nucleotide sequence represented by SEQ ID NO: 5152.

The probes having the nucleotide sequence represented by SEQ ID NOS: 63233, 63234, 63235, 63236, 63237, 63238, 63239, 63240, 63241, 63242, 63243 were selected from the ORFs having the nucleotide sequence represented by SEQ ID NO: 5153.

The probes having the nucleotide sequence represented by SEQ ID NOS: 63244, 63245, 63246, 63247, 63248, 63249, 63250, 63251, 63252, 63253, 63254 were selected from the ORFs having the nucleotide sequence represented by SEQ ID NO: 5154.

The probes having the nucleotide sequence represented by SEQ ID NOS: 63255, 63256, 63257, 63258, 63259, 63260, 63261, 63262, 63263, 63264, 63265 were selected from the ORFs having the nucleotide sequence represented by SEQ ID NO: 5155.

The probes having the nucleotide sequence represented by SEQ ID NOS: 63266, 63267, 63268, 63269, 63270, 63271, 63272, 63273, 63274, 63275, 63276 were selected from the ORFs having the nucleotide sequence represented by SEQ ID NO: 5156.

The probes having the nucleotide sequence represented by SEQ ID NOS: 63277, 63278, 63279, 63280, 63281, 63282, 63283, 63284, 63285, 63286, 63287 were selected from the ORFs having the nucleotide sequence represented by SEQ ID NO: 5157.

The probes having the nucleotide sequence represented by SEQ ID NOS: 63288, 63289, 63290, 63291, 63292, 63293, 63294, 63295, 63296, 63297, 63298 were selected from the ORFs having the nucleotide sequence represented by SEQ ID NO: 5158.

The probes having the nucleotide sequence represented by SEQ ID NOS: 63299, 63300, 63301, 63302, 63303, 63304, 63305, 63306, 63307, 63308, 63309 were selected from the ORFs having the nucleotide sequence represented by SEQ ID NO: 5159.

The probes having the nucleotide sequence represented by SEQ ID NOS: 63310, 63311, 63312, 63313, 63314, 63315, 63316, 63317, 63318, 63319, 63320 were selected from the ORFs having the nucleotide sequence represented by SEQ ID NO: 5160.

The probes having the nucleotide sequence represented by SEQ ID NOS: 63321, 63322, 63323, 63324, 63325, 63326, 63327, 63328, 63329, 63330, 63331 were selected from the ORFs having the nucleotide sequence represented by SEQ ID NO: 5161.

The probes having the nucleotide sequence represented by SEQ ID NOS: 63332, 63333, 63334, 63335, 63336, 63337, 63338, 63339, 63340, 63341, 63342 were selected from the ORFs having the nucleotide sequence represented by SEQ ID NO: 5162.

The probes having the nucleotide sequence represented by SEQ ID NOS: 63343, 63344, 63345, 63346, 63347, 63348, 63349, 63350, 63351, 63352, 63353 were selected from the ORFs having the nucleotide sequence represented by SEQ ID NO: 5163.

The probes having the nucleotide sequence represented by SEQ ID NOS: 63354, 63355, 63356, 63357, 63358, 63359, 63360, 63361, 63362, 63363, 63364 were selected from the ORFs having the nucleotide sequence represented by SEQ ID NO: 5164.

The probes having the nucleotide sequence represented by SEQ ID NOS: 63365, 63366, 63367, 63368, 63369, 63370, 63371, 63372, 63373, 63374, 63375 were selected from the ORFs having the nucleotide sequence represented by SEQ ID NO: 5165.

The probes having the nucleotide sequence represented by SEQ ID NOS: 63376, 63377, 63378, 63379, 63380, 63381, 63382, 63383, 63384, 63385, 63386 were selected from the ORFs having the nucleotide sequence represented by SEQ ID NO: 5166..

The probes having the nucleotide sequence represented by SEQ ID NOS: 63387, 63388, 63389, 63390, 63391, 63392, 63393, 63394, 63395, 63396, 63397 were selected from the ORFs having the nucleotide sequence represented by SEQ ID NO: 5167.

The probes having the nucleotide sequence represented by SEQ ID NOS: 63398, 63399, 63400, 63401, 63402, 63403, 63404, 63405, 63406, 63407, 63408 were selected from the ORFs having the nucleotide sequence represented by SEQ ID NO: 5168.

The probes having the nucleotide sequence represented by SEQ ID NOS: 63409, 63410, 63411, 63412, 63413, 63414, 63415, 63416, 63417, 63418, 63419 were selected from the ORFs having the nucleotide sequence represented by SEQ ID NO: 5169.

The probes having the nucleotide sequence represented by SEQ ID NOS: 63420, 63421, 63422, 63423, 63424, 63425, 63426, 63427, 63428, 63429, 63430 were selected from the ORFs having the nucleotide sequence represented by SEQ ID NO: 5170.

The probes having the nucleotide sequence represented by SEQ ID NOS: 63431, 63432, 63433, 63434, 63435, 63436, 63437, 63438, 63439, 63440, 63441 were selected from the ORFs having the nucleotide sequence represented by SEQ ID NO: 5171.

The probes having the nucleotide sequence represented by SEQ ID NOS: 63442, 63443, 63444, 63445, 63446, 63447, 63448, 63449, 63450, 63451, 63452 were selected from the ORFs having the nucleotide sequence represented by SEQ ID NO: 5172.

The probes having the nucleotide sequence represented by SEQ ID NOS: 63453, 63454, 63455, 63456, 63457, 63458, 63459, 63460, 63461, 63462, 63463 were selected from the ORFs having the nucleotide sequence represented by SEQ ID NO: 5173.

The probes having the nucleotide sequence represented by SEQ ID NOS: 63464, 63465, 63466, 63467, 63468, 63469, 63470, 63471, 63472, 63473, 63474 were selected from the ORFs having the nucleotide sequence represented by SEQ ID NO: 5174.

The probes having the nucleotide sequence represented by SEQ ID NOS: 63475, 63476, 63477, 63478, 63479, 63480, 63481, 63482, 63483, 63484, 63485 were selected from the ORFs having the nucleotide sequence -represented by SEQ ID NO: 5175.

The probes having the nucleotide sequence represented by SEQ ID NOS: 63486, 63487, 63488, 63489, 63490, 63491, 63492, 63493, 63494, 63495, 63496 were selected from the ORFs having the nucleotide sequence represented by SEQ ID NO: 5176.

The probes having the nucleotide sequence represented by SEQ ID NOS: 63497, 63498, 63499, 63500, 63501, 63502, 63503, 63504, 63505, 63506, 63507 were selected from the ORFs having the nucleotide sequence represented by SEQ ID NO: 5177.

The probes having the nucleotide sequence represented by SEQ ID NOS: 63508, 63509, 63510, 63511, 63512, 63513, 63514, 63515, 63516, 63517, 63518 were selected from the ORFs having the nucleotide sequence represented by SEQ ID NO: 5178.

The probes having the nucleotide sequence represented by SEQ ID NOS: 63519, 63520, 63521, 63522, 63523, 63524, 63525, 63526, 63527, 63528, 63529 were selected from the ORFs having the nucleotide sequence represented by SEQ ID NO: 5179.

The probes having the nucleotide sequence represented by SEQ ID NOS: 63530, 63531, 63532, 63533, 63534, 63535, 63536, 63537, 63538, 63539, 63540 were selected from the ORFs having the nucleotide sequence represented by SEQ ID NO: 5180.

The probes having the nucleotide sequence represented by SEQ ID NOS: 63541, 63542, 63543, 63544, 63545, 63546, 63547, 63548, 63549, 63550, 63551 were selected from the ORFs having the nucleotide sequence represented by SEQ ID NO: 5181.

The probes having the nucleotide sequence represented by SEQ ID NOS: 63552, 63553, 63554, 63555, 63556, 63557, 63558, 63559, 63560, 63561, 63562 were selected from the ORFs having the nucleotide sequence represented by SEQ ID NO: 5182.

The probes having the nucleotide sequence represented by SEQ ID NOS: 63563, 63564, 63565, 63566, 63567, 63568, 63569, 63570, 63571, 63572, 63573 were selected from the ORFs having the nucleotide sequence represented by SEQ ID NO: 5183.

The probes having the nucleotide sequence represented by SEQ ID NOS: 63574, 63575, 63576, 63577, 63578, 63579, 63580, 63581, 63582, 63583, 63584 were selected from the ORFs having the nucleotide sequence represented by SEQ ID NO: 5184.

The probes having the nucleotide sequence represented by SEQ ID NOS: 63585, 63586, 63587, 63588, 63589, 63590, 63591, 63592, 63593, 63594, 63595 were selected from the ORFs having the nucleotide sequence represented by SEQ ID NO: 5185.

The probes having the nucleotide sequence represented by SEQ ID NOS: 63596, 63597, 63598, 63599, 63600, 63601, 63602, 63603, 63604, 63605, 63606 were selected from the ORFs having the nucleotide sequence represented by SEQ ID NO: 5186.

The probes having the nucleotide sequence represented by SEQ ID NOS: 63607, 63608, 63609, 63610, 63611, 63612, 63613, 63614, 63615, 63616, 63617 were selected from the ORFs having the nucleotide sequence represented by SEQ ID NO: 5187.

The probes having the nucleotide sequence represented by SEQ ID NOS: 63618, 63619, 63620, 63621, 6.3622, 63623, 63624, 63625, 63626, 63627, 63628 were selected from the ORFs having the nucleotide sequence represented by SEQ ID NO: 5188.

The probes having the nucleotide sequence represented by SEQ ID NOS: 63629, 63630, 63631, 63632, 63633, 63634, 63635, 63636, 63637, 63638, 63639 were selected from the ORFs having the nucleotide sequence represented by SEQ ID NO: 5189.

The probes having the nucleotide sequence represented by SEQ ID NOS: 63640, 63641, 63642, 63643, 63644, 63645, 63646, 63647, 63648, 63649, 63650 were selected from the ORFs having the nucleotide sequence represented by SEQ ID NO: 5190.

The probes having the nucleotide sequence represented by SEQ ID NOS: 63651, 63652, 63653, 63654, 63655, 63656, 63657, 63658, 63659, 63660, 63661 were selected from the ORFs having the nucleotide sequence represented by SEQ ID NO: 5191.

The probes having the nucleotide sequence represented by SEQ ID NOS: 63662, 63663, 63664, 63665, 63666, 63667, 63668, 63669, 63670, 63671, 63672 were selected from the ORFs having the nucleotide sequence represented by SEQ ID NO: 5192.

The probes having the nucleotide sequence represented by SEQ ID NOS: 63673, 63674, 63675, 63676, 63677, 63678, 63679, 63680, 63681, 63682, 63683 were selected from the ORFs having the nucleotide sequence represented by SEQ ID NO: 5193.

The probes having the nucleotide sequence represented by SEQ ID NOS: 63684, 63685, 63686, 63687, 63688, 63689, 63690, 63691, 63692, 63693, 63694 were selected from the ORFs having the nucleotide sequence represented by SEQ ID NO: 5194.

The probes having the nucleotide sequence represented by SEQ ID NOS: 63695, 63696, 63697, 63698, 63699, 63700, 63701, 63702, 63703, 63704, 63705 were selected from the ORFs having the nucleotide sequence represented by SEQ ID NO: 5195.

The probes having the nucleotide sequence represented by SEQ ID NOS: 63706, 63707, 63708, 63709, 63710, 63711, 63712, 63713, 63714, 63715, 63716 were selected from the ORFs having the nucleotide sequence represented by SEQ ID NO: 5196.

The probes having the nucleotide sequence represented by SEQ ID NOS: 63717, 63718, 63719, 63720, 63721, 63722, 63723, 63724, 63725, 63726, 63727 were selected from the ORFs having the nucleotide sequence represented by SEQ ID NO: 5197.

The probes having the nucleotide sequence represented by SEQ ID NOS: 63728, 63729, 63730, 63731, 63732, 63733, 63734, 63735, 63736, 63737, 63738 were selected from the ORFs having the nucleotide sequence represented by SEQ ID NO: 5198.

The probes having the nucleotide sequence represented by SEQ ID NOS: 63739, 63740, 63741, 63742, 63743, 63744, 63745, 63746, 63747, 63748, 63749 were selected from the ORFs having the nucleotide sequence represented by SEQ ID NO: 5199.

The probes having the nucleotide sequence represented by SEQ ID NOS: 63750, 63751, 63752, 63753, 63754, 63755, 63756, 63757, 63758, 63759, 63760 were selected from the ORFs having the nucleotide sequence represented by SEQ ID NO: 5200.

The probes having the nucleotide sequence represented by SEQ ID NOS: 63761, 63762, 63763, 63764, 63765, 63766, 63767, 63768, 63769, 63770, 63771 were selected from the ORFs having the nucleotide sequence represented by SEQ ID NO: 5201.

The probes having the nucleotide sequence represented by SEQ ID NOS: 63772, 63773, 63774, 63775, 63776, 63777, 63778, 63779, 63780, 63781, 63782 were selected from the ORFs having the nucleotide sequence represented by SEQ ID NO: 5202.

The probes having the nucleotide sequence represented by SEQ ID NOS: 63783, 63784, 63785, 63786, 63787, 63788, 63789, 63790, 63791, 63792, 63793 were selected from the ORFs having the nucleotide sequence represented by SEQ ID NO: 5203.

The probes having the nucleotide sequence represented by SEQ ID NOS: 63794, 63795, 63796, 63797, 63798, 63799, 63800, 63801, 63802, 63803, 63804 were selected from the ORFs having the nucleotide sequence represented by SEQ ID NO: 5204.

The probes having the nucleotide sequence represented by SEQ ID NOS: 63805, 63806, 63807, 63808, 63809, 63810, 63811, 63812, 63813, 63814, 63815 were selected from the ORFs having the nucleotide sequence represented by SEQ ID NO: 5205.

The probes having the nucleotide sequence represented by SEQ ID NOS: 63816, 63817, 63818, 63819, 63820, 63821, 63822, 63823, 63824, 63825, 63826 were selected from the ORFs having the nucleotide sequence represented by SEQ ID NO: 5206.

The probes having the nucleotide sequence represented by SEQ ID NOS: 63827, 63828, 63829, 63830, 63831, 63832, 63833, 63834, 63835, 63836, 63837 were selected from the ORFs having the nucleotide sequence represented by SEQ ID NO: 5207.

The probes having the nucleotide sequence represented by SEQ ID NOS: 63838, 63839, 63840, 63841, 63842, 63843, 63844, 63845, 63846, 63847, 63848 were selected from the ORFs having the nucleotide sequence represented by SEQ ID NO: 5208.

The probes having the nucleotide sequence represented by SEQ ID NOS: 63849, 63850, 63851, 63852, 63853, 63854, 63855, 63856, 63857, 63858, 63859 were selected from the ORFs having the nucleotide sequence represented by SEQ ID NO: 5209.

The probes having the nucleotide sequence represented by SEQ ID NOS: 63860, 63861, 63862, 63863, 63864, 63865, 63866, 63867, 63868, 63869, 63870 were selected from the ORFs having the nucleotide sequence represented by SEQ ID NO: 5210.

The probes having the nucleotide sequence represented by SEQ ID NOS: 63871, 63872, 63873, 63874, 63875, 63876, 63877, 63878, 63879, 63880, 63881 were selected from the ORFs having the nucleotide sequence represented by SEQ ID NO: 5211.

The probes having the nucleotide sequence represented by SEQ ID NOS: 63882, 63883, 63884, 63885, 63886, 63887, 63888, 63889, 63890, 63891, 63892 were selected from the ORFs having the nucleotide sequence represented by SEQ ID NO: 5212.

The probes having the nucleotide sequence represented by SEQ ID NOS: 63893, 63894, 63895, 63896, 63897, 63898, 63899, 63900, 63901, 63902, 63903 were selected from the ORFs having the nucleotide sequence represented by SEQ ID NO: 5213.

The probes having the nucleotide sequence represented by SEQ ID NOS: 63904, 63905, 63906, 63907, 63908, 63909, 63910, 63911, 63912, 63913, 63914 were selected from the ORFs having the nucleotide sequence represented by SEQ ID NO: 5214.

The probes having the nucleotide sequence represented by SEQ ID NOS: 63915, 63916, 63917, 63918, 63919, 63920, 63921, 63922, 63923, 63924, 63925 were selected from the ORFs having the nucleotide sequence represented by SEQ ID NO: 5215.

The probes having the nucleotide sequence represented by SEQ ID NOS: 63926, 63927, 63928, 63929, 63930, 63931, 63932, 63933, 63934, 63935, 63936 were selected from the ORFs having the nucleotide sequence represented by SEQ ID NO: 5216.

The probes having the nucleotide sequence represented by SEQ ID NOS: 63937, 63938, 63939, 63940, 63941, 63942, 63943, 63944, 63945, 63946, 63947 were selected from the ORFs having the nucleotide sequence represented by SEQ ID NO: 5217.

The probes having the nucleotide sequence represented by SEQ ID NOS: 63948, 63949, 63950, 63951, 63952, 63953, 63954, 63955, 63956, 63957, 63958 were selected from the ORFs having the nucleotide sequence represented by SEQ ID NO: 5218.

The probes having the nucleotide sequence represented by SEQ ID NOS: 63959, 63960, 63961, 63962, 63963, 63964, 63965, 63966, 63967, 63968, 63969 were selected from the ORFs having the nucleotide sequence represented by SEQ ID NO: 5219.

The probes having the nucleotide sequence represented by SEQ ID NOS: 63970, 63971, 63972, 63973, 63974, 63975, 63976, 63977, 63978, 63979, 63980 were selected from the ORFs having the nucleotide sequence represented by SEQ ID NO: 5220.

The probes having the nucleotide sequence represented by SEQ ID NOS: 63981, 63982, 63983, 63984, 63985, 63986, 63987, 63988, 63989, 63990, 63991 were selected from the ORFs having the nucleotide sequence represented by SEQ ID NO: 5221.

The probes having the nucleotide sequence represented by SEQ ID NOS: 63992, 63993, 63994, 63995, 63996, 63997, 63998, 63999, 64000, 64001, 64002 were selected from the ORFs having the nucleotide sequence represented by SEQ ID NO: 5222.

The probes having the nucleotide sequence represented by SEQ ID NOS: 64003, 64004, 64005, 64006, 64007, 64008, 64009, 64010, 64011, 64012, 64013 were selected from the ORFs having the nucleotide sequence represented by SEQ ID NO: 5223.

The probes having the nucleotide sequence represented by SEQ ID NOS: 64014, 64015, 64016, 64017, 64018, 64019, 64020, 64021, 64022, 64023, 64024 were selected from the ORFs having the nucleotide sequence represented by SEQ ID NO: 5224.

The probes having the nucleotide sequence represented by SEQ ID NOS: 64025, 64026, 64027, 64028, 64029, 64030, 64031, 64032, 64033, 64034, 64035 were selected from the ORFs having the nucleotide sequence represented by SEQ ID NO: 5225.

The probes having the nucleotide sequence represented by SEQ ID NOS: 64036, 64037, 64038, 64039, 64040, 64041, 64042, 64043, 64044, 64045, 64046 were selected from the ORFs having the nucleotide sequence represented by SEQ ID NO: 5226.

The probes having the nucleotide sequence represented by SEQ ID NOS: 64047, 64048, 64049, 64050, 64051, 64052, 64053, 64054, 64055, 64056, 64057 were selected from the ORFs having the nucleotide sequence represented by SEQ ID NO: 5227.

The probes having the nucleotide sequence represented by SEQ ID NOS: 64058, 64059, 64060, 64061, 64062, 64063, 64064, 64065, 64066, 64067, 64068 were selected from the ORFs having the nucleotide sequence represented by SEQ ID NO: 5228.

The probes having the nucleotide sequence represented by SEQ ID NOS: 64069, 64070, 64071, 64072, 64073, 64074, 64075, 64076, 64077, 64078, 64079 were selected from the ORFs having the nucleotide sequence represented by SEQ ID NO: 5229.

The probes having the nucleotide sequence represented by SEQ ID NOS: 64080, 64081, 64082, 64083, 64084, 64085, 64086, 64087, 64088, 64089, 64090 were selected from the ORFs having the nucleotide sequence represented by SEQ ID NO: 5230.

The probes having the nucleotide sequence represented by SEQ ID NOS: 64091, 64092, 64093, 64094, 64095, 64096, 64097, 64098, 64099, 64100, 64101 were selected from the ORFs having the nucleotide sequence represented by SEQ ID NO: 5231.

The probes having the nucleotide sequence represented by SEQ ID NOS: 64102, 64103, 64104, 64105, 64106, 64107, 64108, 64109, 64110, 64111, 64112 were selected from the ORFs having the nucleotide sequence represented by SEQ ID NO: 5232.

The probes having the nucleotide sequence represented by SEQ ID NOS: 64113, 64114, 64115, 64116, 64117, 64118, 64119, 64120, 64121, 64122, 64123 were selected from the ORFs having the nucleotide sequence represented by SEQ ID NO: 5233.

The probes having the nucleotide sequence represented by SEQ ID NOS: 64124, 64125, 64126, 64127, 64128, 64129, 64130, 64131, 64132, 64133, 64134 were selected from the ORFs having the nucleotide sequence represented by SEQ ID NO: 5234.

The probes having the nucleotide sequence represented by SEQ ID NOS: 64135, 64136, 64137, 64138, 64139, 64140, 64141, 64142, 64143, 64144, 64145 were selected from the ORFs having the nucleotide sequence represented by SEQ ID NO: 5235.

The probes having the nucleotide sequence represented by SEQ ID NOS: 64146, 64147, 64148, 64149, 64150, 64151, 64152, 64153, 64154, 64155, 64156 were selected from the ORFs having the nucleotide sequence represented by SEQ ID NO: 5236.

The probes having the nucleotide sequence represented by SEQ ID NOS: 64157, 64158, 64159, 64160, 64161, 64162, 64163, 64164, 64165, 64166, 64167 were selected from the ORFs having the nucleotide sequence represented by SEQ ID NO: 5237.

The probes having the nucleotide sequence represented by SEQ ID NOS: 64168, 64169, 64170, 64171, 64172, 64173, 64174, 64175, 64176, 64177, 64178 were selected from the ORFs having the nucleotide sequence represented by SEQ ID NO: 5238.

The probes having the nucleotide sequence represented by SEQ ID NOS: 64179, 64180, 64181, 64182, 64183, 64184, 64185, 64186, 64187, 64188, 64189 were selected from the ORFs having the nucleotide sequence represented by SEQ ID NO: 5239.

The probes having the nucleotide sequence represented by SEQ ID NOS: 64190, 64191, 64192, 64193, 64194, 64195, 64196, 64197, 64198, 64199, 64200 were selected from the ORFs having the nucleotide sequence represented by SEQ ID NO: 5240.

The probes having the nucleotide sequence represented by SEQ ID NOS: 64201, 64202, 64203, 64204, 64205, 64206, 64207, 64208, 64209, 64210, 64211 were selected from the ORFs having the nucleotide sequence represented by SEQ ID NO: 5241.

The probes having the nucleotide sequence represented by SEQ ID NOS: 64212, 64213, 64214, 64215, 64216, 64217, 64218, 64219, 64220, 64221, 64222 were selected from the ORFs having the nucleotide sequence represented by SEQ ID NO: 5242.

The probes having the nucleotide sequence represented by SEQ ID NOS: 64223, 64224, 64225, 64226, 64227, 64228, 64229, 64230, 64231, 64232, 64233 were selected from the ORFs having the nucleotide sequence represented by SEQ ID NO: 5243.

The probes having the nucleotide sequence represented by SEQ ID NOS: 64234, 64235, 64236, 64237, 64238, 64239, 64240, 64241, 64242, 64243, 64244 were selected from the ORFs having the nucleotide sequence represented by SEQ ID NO: 5244.

The probes having the nucleotide sequence represented by SEQ ID NOS: 64245, 64246, 64247, 64248, 64249, 64250, 64251, 64252, 64253, 64254, 64255 were selected from the ORFs having the nucleotide sequence represented by SEQ ID NO: 5245.

The probes having the nucleotide sequence represented by SEQ ID NOS: 64256, 64257, 64258, 64259, 64260, 64261, 64262, 64263, 64264, 64265, 64266 were selected from the ORFs having the nucleotide sequence represented by SEQ ID NO: 5246.

The probes having the nucleotide sequence represented by SEQ ID NOS: 64267, 64268, 64269, 64270, 64271, 64272, 64273, 64274, 64275, 64276, 64277 were selected from the ORFs having the nucleotide sequence represented by SEQ ID NO: 5247.

The probes having the nucleotide sequence represented by SEQ ID NOS: 64278, 64279, 64280, 64281, 64282, 64283, 64284, 64285, 64286, 64287, 64288 were selected from the ORFs having the nucleotide sequence represented by SEQ ID NO: 5248.

The probes having the nucleotide sequence represented by SEQ ID NOS: 64289, 64290, 64291, 64292, 64293, 64294, 64295, 64296, 64297, 64298, 64299 were selected from the ORFs having the nucleotide sequence represented by SEQ ID NO: 5249.

The probes having the nucleotide sequence represented by SEQ ID NOS: 64300, 64301, 64302, 64303, 64304, 64305, 64306, 64307, 64308, 64309, 64310 were selected from the ORFs having the nucleotide sequence represented by SEQ ID NO: 5250.

The probes having the nucleotide sequence represented by SEQ ID NOS: 64311, 64312, 64313, 64314, 64315, 64316, 64317, 64318, 64319, 64320, 64321 were selected from the ORFs having the nucleotide sequence represented by SEQ ID NO: 5251.

The probes having the nucleotide sequence represented by SEQ ID NOS: 64322, 64323, 64324, 64325, 64326, 64327, 64328, 64329, 64330, 64331, 64332 were selected from the ORFs having the nucleotide sequence represented by SEQ ID NO: 5252.

The probes having the nucleotide sequence represented by SEQ ID NOS: 64333, 64334, 64335, 64336, 64337, 64338, 64339, 64340, 64341, 64342, 64343 were selected from the ORFs having the nucleotide sequence represented by SEQ ID NO: 5253.

The probes having the nucleotide sequence represented by SEQ ID NOS: 64344, 64345, 64346, 64347, 64348, 64349, 64350, 64351, 64352, 64353, 64354 were selected from the ORFs having the nucleotide sequence represented by SEQ ID NO: 5254.

The probes having the nucleotide sequence represented by SEQ ID NOS: 64355, 64356, 64357, 64358, 64359, 64360, 64361, 64362, 64363, 64364, 64365 were selected from the ORFs having the nucleotide sequence represented by SEQ ID NO: 5255.

The probes having the nucleotide sequence represented by SEQ ID NOS: 64366, 64367, 64368, 64369, 64370, 64371, 64372, 64373, 64374, 64375, 64376, 64377, 64378, 64379, 64380, 64381, 64382, 64383, 64384, 64385, 64386, 64387 were selected from the ORFs having the nucleotide sequence represented by SEQ ID NO: 5256.

The probes having the nucleotide sequence represented by SEQ ID NOS: 64388, 64389, 64390, 64391, 64392, 64393, 64394, 64395, 64396, 64397, 64398 were selected from the ORFs having the nucleotide sequence represented by SEQ ID NO: 5257.

The probes having the nucleotide sequence represented by SEQ ID NOS: 64399, 64400, 64401, 64402, 64403, 64404, 64405, 64406, 64407, 64408, 64409 were selected from the ORFs having the nucleotide sequence represented by SEQ ID NO: 5258.

The probes having the nucleotide sequence represented by SEQ ID NOS: 64410, 64411, 64412, 64413, 64414, 64415, 64416, 64417, 64418, 64419, 64420 were selected from the ORFs having the nucleotide sequence represented by SEQ ID NO: 5259.

The probes having the nucleotide sequence represented by SEQ ID NOS: 64421, 64422, 64423, 64424, 64425, 64426, 64427, 64428, 64429, 64430, 64431 were selected from the ORFs having the nucleotide sequence represented by SEQ ID NO: 5260.

The probes having the nucleotide sequence represented by SEQ ID NOS: 64432, 64433, 64434, 64435, 64436, 64437, 64438, 64439, 64440, 64441, 64442 were selected from the ORFs having the nucleotide sequence represented by SEQ ID NO: 5261.

The probes having the nucleotide sequence represented by SEQ ID NOS: 64443, 64444, 64445, 64446, 64447, 64448, 64449, 64450, 64451, 64452, 64453 were selected from the ORFs having the nucleotide sequence represented by SEQ ID NO: 5262.

The probes having the nucleotide sequence represented by SEQ ID NOS: 64454, 64455, 64456, 64457, 64458, 64459, 64460, 64461, 64462, 64463, 64464 were selected from the ORFs having the nucleotide sequence represented by SEQ ID NO: 5263.

The probes having the nucleotide sequence represented by SEQ ID NOS: 64465, 64466, 64467, 64468, 64469, 64470, 64471, 64472, 64473, 64474, 64475 were selected from the ORFs having the nucleotide sequence represented by SEQ ID NO: 5264.

The probes having the nucleotide sequence represented by SEQ ID NOS: 64476, 64477, 64478, 64479, 64480, 64481, 64482, 64483, 64484, 64485, 64486 were selected from the ORFs having the nucleotide sequence represented by SEQ ID NO: 5265.

The probes having the nucleotide sequence represented by SEQ ID NOS: 64487, 64488, 64489, 64490, 64491, 64492, 64493, 64494, 64495, 64496, 64497 were selected from the ORFs having the nucleotide sequence represented by SEQ ID NO: 5266.

The probes having the nucleotide sequence represented by SEQ ID NOS: 64498, 64499, 64500, 64501, 64502, 64503, 64504, 64505, 64506, 64507, 64508 were selected from the ORFs having the nucleotide sequence represented by SEQ ID NO: 5267.

The probes having the nucleotide sequence represented by SEQ ID NOS: 64509, 64510, 64511, 64512, 64513, 64514, 64515, 64516, 64517, 64518, 64519 were selected from the ORFs having the nucleotide sequence represented by SEQ ID NO: 5268.

The probes having the nucleotide sequence represented by SEQ ID NOS: 64520, 64521, 64522, 64523, 64524, 64525, 64526, 64527, 64528, 64529, 64530 were selected from the ORFs having the nucleotide sequence represented by SEQ ID NO: 5269.

The probes having the nucleotide sequence represented by SEQ ID NOS: 64531, 64532, 64533, 64534, 64535, 64536, 64537, 64538, 64539, 64540, 64541 were selected from the ORFs having the nucleotide sequence represented by SEQ ID NO: 5270.

The probes having the nucleotide sequence represented by SEQ ID NOS: 64542, 64543, 64544, 64545, 64546, 64547, 64548, 64549, 64550, 64551, 64552 were selected from the ORFs having the nucleotide sequence represented by SEQ ID NO: 5271.

The probes having the nucleotide sequence represented by SEQ ID NOS: 64553, 64554, 64555, 64556, 64557, 64558, 64559, 64560, 64561, 64562, 64563, 64564, 64565, 64566, 64567, 64568, 64569, 64570, 64571, 64572, 64573, 64574 were selected from the ORFs having the nucleotide sequence represented by SEQ ID NO: 5272.

The probes having the nucleotide sequence represented by SEQ ID NOS: 64575, 64576, 64577, 64578, 64579, 64580, 64581, 64582, 64583, 64584, 64585 were selected from the ORFs having the nucleotide sequence represented by SEQ ID NO: 5273.

The probes having the nucleotide sequence represented by SEQ ID NOS: 64586, 64587, 64588, 64589, 64590, 64591, 64592, 64593, 64594, 64595, 64596 were selected from the ORFs having the nucleotide sequence represented by SEQ ID NO: 5274.

The probes having the nucleotide sequence represented by SEQ ID NOS: 64597, 64598, 64599, 64600, 64601, 64602, 64603, 64604, 64605, 64606, 64607 were selected from the ORFs having the nucleotide sequence represented by SEQ ID NO: 5275.

The probes having the nucleotide sequence represented by SEQ ID NOS: 64608, 64609, 64610, 64611, 64612, 64613, 64614, 64615, 64616, 64617, 64618 were selected from the ORFs having the nucleotide sequence represented by SEQ ID NO: 5276.

The probes having the nucleotide sequence represented by SEQ ID NOS: 64619, 64620, 64621, 64622, 64623, 64624, 64625, 64626, 64627, 64628, 64629 were selected from the ORFs having the nucleotide sequence represented by SEQ ID NO: 5277.

The probes having the-nucleotide sequence represented by SEQ ID NOS: 64630, 64631, 64632, 64633, 64634, 64635, 64636, 64637, 64638, 64639, 64640 were selected from the ORFs having the nucleotide sequence represented by SEQ ID NO: 5278.

The probes having the nucleotide sequence represented by SEQ ID NOS: 64641, 64642, 64643, 64644, 64645, 64646, 64647, 64648, 64649, 64650, 64651 were selected from the ORFs having the nucleotide sequence represented by SEQ ID NO: 5279.

The probes having the nucleotide sequence represented by SEQ ID NOS: 64652, 64653, 64654, 64655, 64656, 64657, 64658, 64659, 64660, 64661, 64662 were selected from the ORFs having the nucleotide sequence represented by SEQ ID NO: 5280.

The probes having the nucleotide sequence represented by SEQ ID NOS: 64663, 64664, 64665, 64666, 64667, 64668, 64669, 64670, 64671, 64672, 64673 were selected from the ORFs having the nucleotide sequence represented by SEQ ID NO: 5281.

The probes having the nucleotide sequence represented by SEQ ID NOS: 64674, 64675, 64676, 64677, 64678, 64679, 64680, 64681, 64682, 64683, 64684 were selected from the ORFs having the nucleotide sequence represented by SEQ ID NO: 5282.

The probes having the nucleotide sequence represented by SEQ ID NOS: 64685, 64686, 64687, 64688, 64689, 64690, 64691, 64692, 64693, 64694, 64695 were selected from the ORFs having the nucleotide sequence represented by SEQ ID NO: 5283.

The probes having the nucleotide sequence represented by SEQ ID NOS: 64696, 64697, 64698, 64699, 64700, 64701, 64702, 64703, 64704, 64705, 64706 were selected from the ORFs having the nucleotide sequence represented by SEQ ID NO: 5284.

The probes having the nucleotide sequence represented by SEQ ID NOS: 64707, 64708, 64709, 64710, 64711, 64712, 64713, 64714, 64715, 64716, 64717 were selected from the ORFs having the nucleotide sequence represented by SEQ ID NO: 5285.

The probes having the nucleotide sequence represented by SEQ ID NOS: 64718, 64719, 64720, 64721, 64722, 64723, 64724, 64725, 64726, 64727, 64728 were selected from the ORFs having the nucleotide sequence represented by SEQ ID NO: 5286.

The probes having the nucleotide sequence represented by SEQ ID NOS: 64729, 64730, 64731, 64732, 64733, 64734, 64735, 64736, 64737, 64738, 64739 were selected from the ORFs having the nucleotide sequence represented by SEQ ID NO: 5287.

The probes having the nucleotide sequence represented by SEQ ID NOS: 64740, 64741, 64742, 64743, 64744, 64745, 64746, 64747, 64748, 64749, 64750 were selected from the ORFs having the nucleotide sequence represented by SEQ ID NO: 5288.

The probes having the nucleotide sequence represented by SEQ ID NOS: 64751, 64752, 64753, 64754, 64755, 64756, 64757, 64758, 64759, 64760, 64761 were selected from the ORFs having the nucleotide sequence represented by SEQ ID NO: 5289.

The probes having the nucleotide sequence represented by SEQ ID NOS: 64762, 64763, 64764, 64765, 64766, 64767, 64768, 64769, 64770, 64771, 64772 were selected from the ORFs having the nucleotide sequence represented by SEQ ID NO: 5290.

The probes having the nucleotide sequence represented by SEQ ID NOS: 64773, 64774, 64775, 64776, 64777, 64778, 64779, 64780, 64781, 64782, 64783 were selected from the ORFs having the nucleotide sequence represented by SEQ ID NO: 5291.

The probes having the nucleotide sequence represented by SEQ ID NOS: 64784, 64785, 64786, 64787, 64788, 64789, 64790, 64791, 64792, 64793, 64794 were selected from the ORFs having the-nucleotide sequence represented by SEQ ID NO: 5292.

The probes having the nucleotide sequence represented by SEQ ID NOS: 64795, 64796, 64797, 64798, 64799, 64800, 64801, 64802, 64803, 64804, 64805 were selected from the ORFs having the nucleotide sequence represented by SEQ ID NO: 5293.

The probes having the nucleotide sequence represented by SEQ ID NOS: 64806, 64807, 64808, 64809, 64810, 64811, 64812, 64813, 64814, 64815, 64816 were selected from the ORFs having the nucleotide sequence represented by SEQ ID NO: 5294.

The probes having the nucleotide sequence represented by SEQ ID NOS: 64817, 64818, 64819, 64820, 64821, 64822, 64823, 64824, 64825, 64826, 64827 were selected from the ORFs having the nucleotide sequence represented by SEQ ID NO: 5295.

The probes having the nucleotide sequence represented by SEQ ID NOS: 64828, 64829, 64830, 64831, 64832, 64833, 64834, 64835, 64836, 64837, 64838 were selected from the ORFs having the nucleotide sequence represented by SEQ ID NO: 5296.

The probes having the nucleotide sequence represented by SEQ ID NOS: 64839, 64840, 64841, 64842, 64843, 64844, 64845, 64846, 64847, 64848, 64849 were selected from the ORFs having the nucleotide sequence represented by SEQ ID NO: 5297.

The probes having the nucleotide sequence represented by SEQ ID NOS: 64850, 64851, 64852, 64853, 64854, 64855, 64856, 64857, 64858, 64859, 64860 were selected from the ORFs having the nucleotide sequence represented by SEQ ID NO: 5298.

The probes having the nucleotide sequence represented by SEQ ID NOS: 64861, 64862, 64863, 64864, 64865, 64866, 64867, 64868, 64869, 64870, 64871 were selected from the ORFs having the nucleotide sequence represented by SEQ ID NO: 5299.

The probes having the nucleotide sequence represented by SEQ ID NOS: 64872, 64873, 64874, 64875, 64876, 64877, 64878, 64879, 64880, 64881, 64882 were selected from the ORFs having the nucleotide sequence represented by SEQ ID NO: 5300.

The probes having the nucleotide sequence represented by SEQ ID NOS: 64883, 64884, 64885, 64886, 64887, 64888, 64889, 64890, 64891, 64892, 64893 were selected from the ORFs having the nucleotide sequence represented by SEQ ID NO: 5301.

The probes having the nucleotide sequence represented by SEQ ID NOS: 64894, 64895, 64896, 64897, 64898, 64899, 64900, 64901, 64902, 64903, 64904 were selected from the ORFs having the nucleotide sequence represented by SEQ ID NO: 5302.

The probes having the nucleotide sequence represented by SEQ ID NOS: 64905, 64906, 64907, 64908, 64909, 64910, 64911, 64912, 64913, 64914, 64915 were selected from the ORFs having the nucleotide sequence represented by SEQ ID NO: 5303.

The probes having the nucleotide sequence represented by SEQ ID NOS: 64916, 64917, 64918, 64919, 64920, 64921, 64922, 64923, 64924, 64925, 64926 were selected from the ORFs having the nucleotide sequence represented by SEQ ID NO: 5304.

The probes having the nucleotide sequence represented by SEQ ID NOS: 64927, 64928, 64929, 64930, 64931, 64932, 64933, 64934, 64935, 64936, 64937 were selected from the ORFs having the nucleotide sequence represented by SEQ ID NO: 5305.

The probes having the nucleotide sequence represented by SEQ ID NOS: 64938, 64939, 64940, 64941, 64942, 64943, 64944, 64945, 64946, 64947, 64948 were selected from the ORFs having the nucleotide sequence represented by SEQ ID NO: 5306.

The probes having the nucleotide sequence represented by SEQ ID NOS: 64949, 64950, 64951, 64952, 64953, 64954, 64955, 64956, 64957, 64958, 64959 were selected from the ORFs having the nucleotide sequence represented by SEQ ID NO: 5307.

The probes having the nucleotide sequence represented by SEQ ID NOS: 64960, 64961, 64962, 64963, 64964, 64965, 64966, 64967, 64968, 64969, 64970 were selected from the ORFs having the nucleotide sequence represented by SEQ ID NO: 5308.

The probes having the nucleotide sequence represented by SEQ ID NOS: 64971, 64972, 64973, 64974, 64975, 64976, 64977, 64978, 64979, 64980, 64981 were selected from the ORFs having the nucleotide sequence represented by SEQ ID NO: 5309.

The probes having the nucleotide sequence represented by SEQ ID NOS: 64982, 64983, 64984, 64985, 64986, 64987, 64988, 64989, 64990, 64991, 64992 were selected from the ORFs having the nucleotide sequence represented by SEQ ID NO: 5310.

The probes having the nucleotide sequence represented by SEQ ID NOS: 64993, 64994, 64995, 64996, 64997, 64998, 64999, 65000, 65001, 65002, 65003 were selected from the ORFs having the nucleotide sequence represented by SEQ ID NO: 5311.

The probes having the nucleotide sequence represented by SEQ ID NOS: 65004, 65005, 65006, 65007, 65008, 65009, 65010, 65011, 65012, 65013, 65014 were selected from the ORFs having the nucleotide sequence represented by SEQ ID NO: 5312.

The probes having the nucleotide sequence represented by SEQ ID NOS: 65015, 65016, 65017, 65018, 65019, 65020, 65021, 65022, 65023, 65024, 65025 were selected from the ORFs having the nucleotide sequence represented by SEQ ID NO: 5313.

The probes having the nucleotide sequence represented by SEQ ID NOS: 65026, 65027, 65028, 65029, 65030, 65031, 65032, 65033, 65034, 65035, 65036 were selected from the ORFs having the nucleotide sequence represented by SEQ ID NO: 5314.

The probes having the nucleotide sequence represented by SEQ ID NOS: 65037, 65038, 65039, 65040, 65041, 65042, 65043, 65044, 65045, 65046, 65047 were selected from the ORFs having the nucleotide sequence represented by SEQ ID NO: 5315.

The probes having the nucleotide sequence represented by SEQ ID NOS: 65048, 65049, 65050, 65051, 65052, 65053, 65054, 65055, 65056, 65057, 65058 were selected from the ORFs having the nucleotide sequence represented by SEQ ID NO: 5316.

The probes having the nucleotide sequence represented by SEQ ID NOS: 65059, 65060, 65061, 65062, 65063, 65064, 65065, 65066, 65067, 65068, 65069 were selected from the ORFs having the nucleotide sequence represented by SEQ ID NO: 5317.

The probes having the nucleotide sequence represented by SEQ ID NOS: 65070, 65071, 65072, 65073, 65074, 65075, 65076, 65077, 65078, 65079, 65080 were selected from the ORFs having the nucleotide sequence represented by SEQ ID NO: 5318.

The probes having the nucleotide sequence represented by SEQ ID NOS: 65081, 65082, 65083, 65084, 65085, 65086, 65087, 65088, 65089, 65090, 65091 were selected from the ORFs having the nucleotide sequence represented by SEQ ID NO: 5319.

The probes having the nucleotide sequence represented by SEQ ID NOS: 65092, 65093, 65094, 65095, 65096, 65097, 65098, 65099, 65100, 65101, 65102 were selected from the ORFs having the nucleotide sequence represented by SEQ ID NO: 5320.

The probes having the nucleotide sequence represented by SEQ ID NOS: 65103, 65104, 65105, 65106, 65107, 65108, 65109, 65110, 65111, 65112, 65113 were selected from the ORFs having the nucleotide sequence represented by SEQ ID NO: 5321.

The probes having the nucleotide sequence represented by SEQ ID NOS: 65114, 65115, 65116, 65117, 65118, 65119, 65120, 65121, 65122, 65123, 65124 were selected from the ORFs having the nucleotide sequence represented by SEQ ID NO: 5322.

The probes having the nucleotide sequence represented by SEQ ID NOS: 65125, 65126, 65127, 65128, 65129, 65130, 65131, 65132, 65133, 65134, 65135 were selected from the ORFs having the nucleotide sequence represented by SEQ ID NO: 5323.

The probes having the nucleotide sequence represented by SEQ ID NOS: 65136, 65137, 65138, 65139, 65140, 65141, 65142, 65143, 65144, 65145, 65146 were selected from the ORFs having the nucleotide sequence represented by SEQ ID NO: 5324.

The probes having the nucleotide sequence represented by SEQ ID NOS: 65147, 65148, 65149, 65150, 65151, 65152, 65153, 65154, 65155, 65156, 65157 were selected from the ORFs having the nucleotide sequence represented by SEQ ID NO: 5325.

The probes having the nucleotide sequence represented by SEQ ID NOS: 65158, 65159, 65160, 65161, 65162, 65163, 65164, 65165, 65166, 65167, 65168 were selected from the ORFs having the nucleotide sequence represented by SEQ ID NO: 5326.

The probes having the nucleotide sequence represented by SEQ ID NOS: 65169, 65170, 65171, 65172, 65173, 65174, 65175, 65176, 65177, 65178, 65179 were selected from the ORFs having the nucleotide sequence represented by SEQ ID NO: 5327.

The probes having the nucleotide sequence represented by SEQ ID NOS: 65180, 65181, 65182, 65183, 65184, 65185, 65186, 65187, 65188, 65189, 65190 were selected from the ORFs having the nucleotide sequence represented by SEQ ID NO: 5328.

The probes having the nucleotide sequence represented by SEQ ID NOS: 65191, 65192, 65193, 65194, 65195, 65196, 65197, 65198, 65199, 65200, 65201 were selected from the ORFs having the nucleotide sequence represented by SEQ ID NO: 5329.

The probes having the nucleotide sequence represented by SEQ ID NOS: 65202, 65203, 65204, 65205, 65206, 65207, 65208, 65209, 65210, 65211, 65212 were selected from the ORFs having the nucleotide sequence represented by SEQ ID NO: 5330.

The probes having the nucleotide sequence represented by SEQ ID NOS: 65213, 65214, 65215, 65216, 65217, 65218, 65219, 65220, 65221, 65222, 65223 were selected from the ORFs having the nucleotide sequence represented by SEQ ID NO: 5331.

The probes having the nucleotide sequence represented by SEQ ID NOS: 65224, 65225, 65226, 65227, 65228, 65229, 65230, 65231, 65232, 65233, 65234 were selected from the ORFs having the nucleotide sequence represented by SEQ ID NO: 5332.

The probes having the nucleotide sequence represented by SEQ ID NOS: 65235, 65236, 65237, 65238, 65239, 65240, 65241, 65242, 65243, 65244, 65245 were selected from the ORFs having the nucleotide sequence represented by SEQ ID NO: 5333.

The probes having the nucleotide sequence represented by SEQ ID NOS: 65246, 65247, 65248, 65249, 65250, 65251, 65252, 65253, 65254, 65255, 65256 were selected from the ORFs having the nucleotide sequence represented by SEQ ID NO: 5334.

The probes having the nucleotide sequence represented by SEQ ID NOS: 65257, 65258, 65259, 65260, 65261, 65262, 65263, 65264, 65265, 65266, 65267 were selected from the ORFs having the nucleotide sequence represented by SEQ ID NO: 5335.

The probes having the nucleotide sequence represented by SEQ ID NOS: 65268, 65269, 65270, 65271, 65272, 65273, 65274, 65275, 65276, 65277, 65278 were selected from the ORFs having the nucleotide sequence represented by SEQ ID NO: 5336.

The probes having the nucleotide sequence represented by SEQ ID NOS: 65279, 65280, 65281, 65282, 65283, 65284, 65285, 65286, 65287, 65288, 65289 were selected from the ORFs having the nucleotide sequence represented by SEQ ID NO: 5337.

The probes having the nucleotide sequence represented by SEQ ID NOS: 65290, 65291, 65292, 65293, 65294, 65295, 65296, 65297, 65298, 65299, 65300 were selected from the ORFs having the nucleotide sequence represented by SEQ ID NO: 5338.

The probes having the nucleotide sequence represented by SEQ ID NOS: 65301, 65302, 65303, 65304, 65305, 65306, 65307, 65308, 65309, 65310, 65311 were selected from the ORFs having the nucleotide sequence represented by SEQ ID NO: 5339.

The probes having the nucleotide sequence represented by SEQ ID NOS: 65312, 65313, 65314, 65315, 65316, 65317, 65318, 65319, 65320, 65321, 65322 were selected from the ORFs having the nucleotide sequence represented by SEQ ID NO: 5340.

The probes having the nucleotide sequence represented by SEQ ID NOS: 65323, 65324, 65325, 65326, 65327, 65328, 65329, 65330, 65331, 65332, 65333 were selected from the ORFs having the nucleotide sequence represented by SEQ ID NO: 5341.

The probes having the nucleotide sequence represented by SEQ ID NOS: 65334, 65335, 65336, 65337, 65338, 65339, 65340, 65341, 65342, 65343, 65344 were selected from the ORFs having the nucleotide sequence represented by SEQ ID NO: 5342.

The probes having the nucleotide sequence represented by SEQ ID NOS: 65345, 65346, 65347, 65348, 65349, 65350, 65351, 65352, 65353, 65354, 65355 were selected from the ORFs having the nucleotide sequence represented by SEQ ID NO: 5343.

The probes having the nucleotide sequence represented by SEQ ID NOS: 65356, 65357, 65358, 65359, 65360, 65361, 65362, 65363, 65364, 65365, 65366 were selected from the ORFs having the nucleotide sequence represented by SEQ ID NO: 5344.

The probes having the nucleotide sequence represented by SEQ ID NOS: 65367, 65368, 65369, 65370, 65371, 65372, 65373, 65374, 65375, 65376, 65377 were selected from the ORFs having the nucleotide sequence represented by SEQ ID NO: 5345.

The probes having the nucleotide sequence represented by SEQ ID NOS: 65378, 65379, 65380, 65381, 65382, 65383, 65384, 65385, 65386, 65387, 65388 were selected from the ORFs having the nucleotide sequence represented by SEQ ID NO: 5346.

The probes having the nucleotide sequence represented by SEQ ID NOS: 65389, 65390, 65391, 65392, 65393, 65394, 65395, 65396, 65397, 65398, 65399 were selected from the ORFs having the nucleotide sequence represented by SEQ ID NO: 5347.

The probes having the nucleotide sequence represented by SEQ ID NOS: 65400, 65401, 65402, 65403, 65404, 65405, 65406, 65407, 65408, 65409, 65410 were selected from the ORFs having the nucleotide sequence represented by SEQ ID NO: 5348.

The probes having the nucleotide sequence represented by SEQ ID NOS: 65411, 65412, 65413, 65414, 65415, 65416, 65417, 65418, 65419, 65420, 65421 were selected from the ORFs having the nucleotide sequence represented by SEQ ID NO: 5349.

The probes having the nucleotide sequence represented by SEQ ID NOS: 65422, 65423, 65424, 65425, 65426, 65427, 65428, 65429, 65430, 65431, 65432 were selected from the ORFs having the nucleotide sequence represented by SEQ ID NO: 5350.

The probes having the nucleotide sequence represented by SEQ ID NOS: 65433, 65434, 65435, 65436, 65437, 65438, 65439, 65440, 65441, 65442, 65443 were selected from the ORFs having the nucleotide sequence represented by SEQ ID NO: 5351.

The probes having the nucleotide sequence represented by SEQ ID NOS: 65444, 65445, 65446, 65447, 65448, 65449, 65450, 65451, 65452, 65453, 65454 were selected from the ORFs having the nucleotide sequence represented by SEQ ID NO: 5352.

The probes having the nucleotide sequence represented by SEQ ID NOS: 65455, 65456, 65457, 65458, 65459, 65460, 65461, 65462, 65463, 65464, 65465 were selected from the ORFs having the nucleotide sequence represented by SEQ ID NO: 5353.

The probes having the nucleotide sequence represented by SEQ ID NOS: 65466, 65467, 65468, 65469, 65470, 65471, 65472, 65473, 65474, 65475, 65476 were selected from the ORFs having the nucleotide sequence represented by SEQ ID NO: 5354.

The probes having the nucleotide sequence represented by SEQ ID NOS: 65477, 65478, 65479, 65480, 65481, 65482, 65483, 65484, 65485, 65486, 65487 were selected from the ORFs having the nucleotide sequence represented by SEQ ID NO: 5355.

The probes having the nucleotide sequence represented by SEQ ID NOS: 65488, 65489, 65490, 65491, 65492, 65493,

65494, 65495, 65496, 65497, 65498 were selected from the ORFs having the nucleotide sequence represented by SEQ ID NO: 5356.

The probes having the nucleotide sequence represented by SEQ ID NOS: 65499, 65500, 65501, 65502, 65503, 65504, 65505, 65506, 65507, 65508, 65509 were selected from the ORFs having the nucleotide sequence represented by SEQ ID NO: 5357.

The probes having the nucleotide sequence represented by SEQ ID NOS: 65510, 65511, 65512, 65513, 65514, 65515, 65516, 65517, 65518, 65519, 65520 were selected from the ORFs having the nucleotide sequence represented by SEQ ID NO: 5358.

The probes having the nucleotide sequence represented by SEQ ID NOS: 65521, 65522, 65523, 65524, 65525, 65526, 65527, 65528, 65529, 65530, 65531 were selected from the ORFs having the nucleotide sequence represented by SEQ ID NO: 5359.

The probes having the nucleotide sequence represented by SEQ ID NOS: 65532, 65533, 65534, 65535, 65536, 65537, 65538, 65539, 65540, 65541, 65542 were selected from the ORFs having the nucleotide sequence represented by SEQ ID NO: 5360.

The probes having the nucleotide sequence represented by SEQ ID NOS: 65543, 65544, 65545, 65546, 65547, 65548, 65549, 65550, 65551, 65552, 65553 were selected from the ORFs having the nucleotide sequence represented by SEQ ID NO: 5361.

The probes having the nucleotide sequence represented by SEQ ID NOS: 65554, 65555, 65556, 65557, 65558, 65559, 65560, 65561, 65562, 65563, 65564 were selected from the ORFs having the nucleotide sequence represented by SEQ ID NO: 5362.

The probes having the nucleotide sequence represented by SEQ ID NOS: 65565, 65566, 65567, 65568, 65569, 65570, 65571, 65572, 65573, 65574, 65575 were selected from the ORFs having the nucleotide sequence represented by SEQ ID NO: 5363.

The probes having the nucleotide sequence represented by SEQ ID NOS: 65576, 65577, 65578, 65579, 65580, 65581, 65582, 65583, 65584, 65585, 65586 were selected from the ORFs having the nucleotide sequence represented by SEQ ID NO: 5364.

The probes having the nucleotide sequence represented by SEQ ID NOS: 65587, 65588, 65589, 65590, 65591, 65592, 65593, 65594, 65595, 65596, 65597 were selected from the ORFs having the nucleotide sequence represented by SEQ ID NO: 5365.

The probes having the nucleotide sequence represented by SEQ ID NOS: 65598, 65599, 65600, 65601, 65602, 65603, 65604, 65605, 65606, 65607, 65608 were selected from the ORFs having the nucleotide sequence represented by SEQ ID NO: 5366.

The probes having the nucleotide sequence represented by SEQ ID NOS: 65609, 65610, 65611, 65612, 65613, 65614, 65615, 65616, 65617, 65618, 65619 were selected from the ORFs having the nucleotide sequence represented by SEQ ID NO: 5367.

The probes having the nucleotide sequence represented by SEQ ID NOS: 65620, 65621, 65622, 65623, 65624, 65625, 65626, 65627, 65628, 65629, 65630 were selected from the ORFs having the nucleotide sequence represented by SEQ ID NO: 5368.

The probes having the nucleotide sequence represented by SEQ ID NOS: 65631, 65632, 65633, 65634, 65635, 65636, 65637, 65638, 65639, 65640, 65641 were selected from the ORFs having the nucleotide sequence represented by SEQ ID NO: 5369.

The probes having the nucleotide sequence represented by SEQ ID NOS: 65642, 65643, 65644, 65645, 65646, 65647, 65648, 65649, 65650, 65651, 65652 were selected from the ORFs having the nucleotide sequence represented by SEQ ID NO: 5370.

The probes having the nucleotide sequence represented by SEQ ID NOS: 65653, 65654, 65655, 65656, 65657, 65658, 65659, 65660, 65661, 65662, 65663 were selected from the ORFs having the nucleotide sequence represented by SEQ ID NO: 5371.

The probes having the nucleotide sequence represented by SEQ ID NOS: 65664, 65665, 65666, 65667, 65668, 65669, 65670, 65671, 65672, 65673, 65674 were selected from the ORFs having the nucleotide sequence represented by SEQ ID NO: 5372.

The probes having the nucleotide sequence represented by SEQ ID NOS: 65675, 65676, 65677, 65678, 65679, 65680, 65681, 65682, 65683, 65684, 65685 were selected from the ORFs having the nucleotide sequence represented by SEQ ID NO: 5373.

The probes having the nucleotide sequence represented by SEQ ID NOS: 65686, 65687, 65688, 65689, 65690, 65691, 65692, 65693, 65694, 65695, 65696 were selected from the ORFs having the nucleotide sequence represented by SEQ ID NO: 5374.

The probes having the nucleotide sequence represented by SEQ ID NOS: 65697, 65698, 65699, 65700, 65701, 65702, 65703, 65704, 65705, 65706, 65707 were selected from the ORFs having the nucleotide sequence represented by SEQ ID NO: 5375.

The probes having the nucleotide sequence represented by SEQ ID NOS: 65708, 65709, 65710, 65711, 65712, 65713, 65714, 65715, 65716, 65717, 65718 were selected from the ORFs having the nucleotide sequence represented by SEQ ID NO: 5376.

The probes having the nucleotide sequence represented by SEQ ID NOS: 65719, 65720, 65721, 65722, 65723, 65724, 65725, 65726, 65727, 65728, 65729 were selected from the ORFs having the nucleotide sequence represented by SEQ ID NO: 5377.

The probes having the nucleotide sequence represented by SEQ ID NOS: 65730, 65731, 65732, 65733, 65734, 65735, 65736, 65737, 65738, 65739, 65740 were selected from the ORFs having the nucleotide sequence represented by SEQ ID NO: 5378.

The probes having the nucleotide sequence represented by SEQ ID NOS: 65741, 65742, 65743, 65744, 65745, 65746, 65747, 65748, 65749, 65750, 65751 were selected from the ORFs having the nucleotide sequence represented by SEQ ID NO: 5379.

The probes having the nucleotide sequence represented by SEQ ID NOS: 65752, 65753, 65754, 65755, 65756, 65757, 65758, 65759, 65760, 65761, 65762 were selected from the ORFs having the nucleotide sequence represented by SEQ ID NO: 5380.

The probes having the nucleotide sequence represented by SEQ ID NOS: 65763, 65764, 65765, 65766, 65767, 65768, 65769, 65770, 65771, 65772, 65773 were selected from the ORFs having the nucleotide sequence represented by SEQ ID NO: 5381.

The probes having the nucleotide sequence represented by SEQ ID NOS: 65774, 65775, 65776, 65777, 65778, 65779, 65780, 65781, 65782, 65783, 65784 were selected from the ORFs having the nucleotide sequence represented by SEQ ID NO: 5382.

The probes having the nucleotide sequence represented by SEQ ID NOS: 65785, 65786, 65787, 65788, 65789, 65790, 65791, 65792, 65793, 65794, 65795 were selected from the ORFs having the nucleotide sequence represented by SEQ ID NO: 5383.

The probes having the nucleotide sequence represented by SEQ ID NOS: 65796, 65797, 65798, 65799, 65800, 65801, 65802, 65803, 65804, 65805, 65806 were selected from the ORFs having the nucleotide sequence represented by SEQ ID NO: 5384.

The probes having the nucleotide sequence represented by SEQ ID NOS: 65807, 65808, 65809, 65810, 65811, 65812, 65813, 65814, 65815, 65816, 65817 were selected from the ORFs having the nucleotide sequence represented by SEQ ID NO: 5385.

The probes having the nucleotide sequence represented by SEQ ID NOS: 65818, 65819, 65820, 65821, 65822, 65823, 65824, 65825, 65826, 65827, 65828 were selected from the ORFs having the nucleotide sequence represented by SEQ ID NO: 5386.

The probes having the nucleotide sequence represented by SEQ ID NOS: 65829, 65830, 65831, 65832, 65833, 65834, 65835, 65836, 65837, 65838, 65839 were selected from the ORFs having the nucleotide sequence represented by SEQ ID NO: 5387.

The probes having the nucleotide sequence represented by SEQ ID NOS: 65840, 65841, 65842, 65843, 65844, 65845, 65846, 65847, 65848, 65849, 65850 were selected from the ORFs having the nucleotide sequence represented by SEQ ID NO: 5388.

The probes having the nucleotide sequence represented by SEQ ID NOS: 65851, 65852, 65853, 65854, 65855, 65856, 65857, 65858, 65859, 65860, 65861 were selected from the ORFs having the nucleotide sequence represented by SEQ ID NO: 5389.

The probes having the nucleotide sequence represented by SEQ ID NOS: 65862, 65863, 65864, 65865, 65866, 65867, 65868, 65869, 65870, 65871, 65872 were selected from the ORFs having the nucleotide sequence represented by SEQ ID NO: 5390.

The probes having the nucleotide sequence represented by SEQ ID NOS: 65873, 65874, 65875, 65876, 65877, 65878, 65879, 65880, 65881, 65882, 65883 were selected from the ORFs having the nucleotide sequence represented by SEQ ID NO: 5391.

The probes having the nucleotide sequence represented by SEQ ID NOS: 65884, 65885, 65886, 65887, 65888, 65889, 65890, 65891, 65892, 65893, 65894 were selected from the ORFs having the nucleotide sequence represented by SEQ ID NO: 5392.

The probes having the nucleotide sequence represented by SEQ ID NOS: 65895, 65896, 65897, 65898, 65899, 65900, 65901, 65902, 65903, 65904, 65905 were selected from the ORFs having the nucleotide sequence represented by SEQ ID NO: 5393.

The probes having the nucleotide sequence represented by SEQ ID NOS: 65906, 65907, 65908, 65909, 65910, 65911, 65912, 65913, 65914, 65915, 65916 were selected from the ORFs having the nucleotide sequence represented by SEQ ID NO: 5394.

The probes having the nucleotide sequence represented by SEQ ID NOS: 65917, 65918, 65919, 65920, 65921, 65922, 65923, 65924, 65925, 65926, 65927 were selected from the ORFs having the nucleotide sequence represented by SEQ ID NO: 5395.

The probes having the nucleotide sequence represented by SEQ ID NOS: 65928, 65929, 65930, 65931, 65932, 65933, 65934, 65935, 65936, 65937, 65938 were selected from the ORFs having the nucleotide sequence represented by SEQ ID NO: 5396.

The probes having the nucleotide sequence represented by SEQ ID NOS: 65939, 65940, 65941, 65942, 65943, 65944, 65945, 65946, 65947, 65948, 65949 were selected from the ORFs having the nucleotide sequence represented by SEQ ID NO: 5397.

The probes having the nucleotide sequence represented by SEQ ID NOS: 65950, 65951, 65952, 65953, 65954, 65955, 65956, 65957, 65958, 65959, 65960 were selected from the ORFs having the nucleotide sequence represented by SEQ ID NO: 5398.

The probes having the nucleotide sequence represented by SEQ ID NOS: 65961, 65962, 65963, 65964, 65965, 65966, 65967, 65968, 65969, 65970, 65971 were selected from the ORFs having the nucleotide sequence represented by SEQ ID NO: 5399.

The probes having the nucleotide sequence represented by SEQ ID NOS: 65972, 65973, 65974, 65975, 65976, 65977, 65978, 65979, 65980, 65981, 65982 were selected from the ORFs having the nucleotide sequence represented by SEQ ID NO: 5400.

The probes having the nucleotide sequence represented by SEQ ID NOS: 65983, 65984, 65985, 65986, 65987, 65988, 65989, 65990, 65991, 65992, 65993 were selected from the ORFs having the nucleotide sequence represented by SEQ ID NO: 5401.

The probes having the nucleotide sequence represented by SEQ ID NOS: 65994, 65995, 65996, 65997, 65998, 65999, 66000, 66001, 66002, 66003, 66004 were selected from the ORFs having the nucleotide sequence represented by SEQ ID NO: 5402.

The probes having the nucleotide sequence represented by SEQ ID NOS: 66005, 66006, 66007, 66008, 66009, 66010, 66011, 66012, 66013, 66014, 66015 were selected from the ORFs having the nucleotide sequence represented by SEQ ID NO: 5403.

The probes having the nucleotide sequence represented by SEQ ID NOS: 66016, 66017, 66018, 66019, 66020, 66021, 66022, 66023, 66024, 66025, 66026 were selected from the ORFs having the nucleotide sequence represented by SEQ ID NO: 5404.

The probes having the nucleotide sequence represented by SEQ ID NOS: 66027, 66028, 66029, 66030, 66031, 66032, 66033, 66034, 66035, 66036, 66037 were selected from the ORFs having the nucleotide sequence represented by SEQ ID NO: 5405.

The probes having the nucleotide sequence represented by SEQ ID NOS: 66038, 66039, 66040, 66041, 66042, 66043, 66044, 66045, 66046, 66047, 66048 were selected from the ORFs having the nucleotide sequence represented by SEQ ID NO: 5406.

The probes having the nucleotide sequence represented by SEQ ID NOS: 66049, 66050, 66051, 66052, 66053, 66054, 66055, 66056, 66057, 66058, 66059 were selected from the ORFs having the nucleotide sequence represented by SEQ ID NO: 5407.

The probes having the nucleotide sequence represented by SEQ ID NOS: 66060, 66061, 66062, 66063, 66064, 66065, 66066, 66067, 66068, 66069, 66070 were selected from the ORFs having the nucleotide sequence represented by SEQ ID NO: 5408.

The probes having the nucleotide sequence represented by SEQ ID NOS: 66071, 66072, 66073, 66074, 66075, 66076, 66077, 66078, 66079, 66080, 66081 were selected from the ORFs having the nucleotide sequence represented by SEQ ID NO: 5409.

The probes having the nucleotide sequence represented by SEQ ID NOS: 66082, 66083, 66084, 66085, 66086, 66087, 66088, 66089, 66090, 66091, 66092 were selected from the ORFs having the nucleotide sequence represented by SEQ ID NO: 5410.

The probes having the nucleotide sequence represented by SEQ ID NOS: 66093, 66094, 66095, 66096, 66097, 66098, 66099, 66100, 66101, 66102, 66103, 66104, 66105, 66106, 66107, 66108, 66109, 66110, 66111, 66112, 66113, 66114 were selected from the ORFs having the nucleotide sequence represented by SEQ ID NO: 5411.

The probes having the nucleotide sequence represented by SEQ ID NOS: 66115, 66116, 66117, 66118, 66119, 66120, 66121, 66122, 66123, 66124, 66125 were selected from the ORFs having the nucleotide sequence represented by SEQ ID NO: 5412.

The probes having the nucleotide sequence represented by SEQ ID NOS: 66126, 66127, 66128, 66129, 66130, 66131, 66132, 66133, 66134, 66135, 66136 were selected from the ORFs having the nucleotide sequence represented by SEQ ID NO: 5413.

The probes having the nucleotide sequence represented by SEQ ID NOS: 66137, 66138, 66139, 66140, 66141, 66142, 66143, 66144, 66145, 66146, 66147 were selected from the ORFs having the nucleotide sequence represented by SEQ ID NO: 5414.

The probes having the nucleotide sequence represented by SEQ ID NOS: 66148, 66149, 66150, 66151, 66152, 66153, 66154, 66155, 66156, 66157, 66158 were selected from the ORFs having the nucleotide sequence represented by SEQ ID NO: 5415.

The probes having the nucleotide sequence represented by SEQ ID NOS: 66159, 66160, 66161, 66162, 66163, 66164, 66165, 66166, 66167, 66168, 66169 were selected from the ORFs having the nucleotide sequence represented by SEQ ID NO: 5416.

The probes having the nucleotide sequence represented by SEQ ID NOS: 66170, 66171, 66172, 66173, 66174, 66175, 66176, 66177, 66178, 66179, 66180 were selected from the ORFs having the nucleotide sequence represented by SEQ ID NO: 5417.

The probes having the nucleotide sequence represented by SEQ ID NOS: 66181, 66182, 66183, 66184, 66185, 66186, 66187, 66188, 66189, 66190, 66191 were selected from the ORFs having the nucleotide sequence represented by SEQ ID NO: 5418.

The probes having the nucleotide sequence represented by SEQ ID NOS: 66192, 66193, 66194, 66195, 66196, 66197, 66198, 66199, 66200, 66201, 66202 were selected from the ORFs having the nucleotide sequence represented by SEQ ID NO: 5419.

The probes having the nucleotide sequence represented by SEQ ID NOS: 66203, 66204, 66205, 66206, 66207, 66208, 66209, 66210, 66211, 66212, 66213 were selected from the ORFs having the nucleotide sequence represented by SEQ ID NO: 5420.

The probes having the nucleotide sequence represented by SEQ ID NOS: 66214, 66215, 66216, 66217, 66218, 66219, 66220, 66221, 66222, 66223, 66224 were selected from the ORFs having the nucleotide sequence represented by SEQ ID NO: 5421.

The probes having the nucleotide sequence represented by SEQ ID NOS: 66225, 66226, 66227, 66228, 66229, 66230, 66231, 66232, 66233, 66234, 66235 were selected from the ORFs having the nucleotide sequence represented by SEQ ID NO: 5422.

The probes having the nucleotide sequence represented by SEQ ID NOS: 66236, 66237, 66238, 66239, 66240, 66241, 66242, 66243, 66244, 66245, 66246 were selected from the ORFs having the nucleotide sequence represented by SEQ ID NO: 5423.

The probes having the nucleotide sequence represented by SEQ ID NOS: 66247, 66248, 66249, 66250, 66251, 66252, 66253, 66254, 66255, 66256, 66257 were selected from the ORFs having the nucleotide sequence represented by SEQ ID NO: 5424.

The probes having the nucleotide sequence represented by SEQ ID NOS: 66258, 66259, 66260, 66261, 66262, 66263, 66264, 66265, 66266, 66267, 66268 were selected from the ORFs having the nucleotide sequence represented by SEQ ID NO: 5425.

The probes having the nucleotide sequence represented by SEQ ID NOS: 66269, 66270, 66271, 66272, 66273, 66274, 66275, 66276, 66277, 66278, 66279 were selected from the ORFs having the nucleotide sequence represented by SEQ ID NO: 5426.

The probes having the nucleotide sequence represented by SEQ ID NOS: 66280, 66281, 66282, 66283, 66284, 66285, 66286, 66287, 66288, 66289, 66290 were selected from the ORFs having the nucleotide sequence represented by SEQ ID NO: 5427.

The probes having the nucleotide sequence represented by SEQ ID NOS: 66291, 66292, 66293, 66294, 66295, 66296, 66297, 66298, 66299, 66300, 66301 were selected from the ORFs having the nucleotide sequence represented by SEQ ID NO: 5428.

The probes having the nucleotide sequence represented by SEQ ID NOS: 66302, 66303, 66304, 66305, 66306, 66307, 66308, 66309, 66310, 66311, 66312 were selected from the ORFs having the nucleotide sequence represented by SEQ ID NO: 5429.

The probes having the nucleotide sequence represented by SEQ ID NOS: 66313, 66314, 66315, 66316, 66317, 66318, 66319, 66320, 66321, 66322, 66323 were selected from the ORFs having the nucleotide sequence represented by SEQ ID NO: 5430.

The probes having the nucleotide sequence represented by SEQ ID NOS: 66324, 66325, 66326, 66327, 66328, 66329, 66330, 66331, 66332, 66333, 66334 were selected from the ORFs having the nucleotide sequence represented by SEQ ID NO: 5431.

The probes having the nucleotide sequence represented by SEQ ID NOS: 66335, 66336, 66337, 66338, 66339, 66340, 66341, 66342, 66343, 66344, 66345 were selected from the ORFs having the nucleotide sequence represented by SEQ ID NO: 5432.

The probes having the nucleotide sequence represented by SEQ ID NOS: 66346, 66347, 66348, 66349, 66350, 66351, 66352, 66353, 66354, 66355, 66356 were selected from the ORFs having the nucleotide sequence represented by SEQ ID NO: 5433.

The probes having the nucleotide sequence represented by SEQ ID NOS: 66357, 66358, 66359, 66360, 66361,,66362, 66363, 66364, 66365, 66366, 66367 were selected from the ORFs having the nucleotide sequence represented by SEQ ID NO: 5434.

The probes having the nucleotide sequence represented by SEQ ID NOS: 66368, 66369, 66370, 66371, 66372, 66373, 66374, 66375, 66376, 66377, 66378 were selected from the ORFs having the nucleotide sequence represented by SEQ ID NO: 5435.

The probes having the nucleotide sequence represented by SEQ ID NOS: 66379, 66380, 66381, 66382, 66383, 66384, 66385, 66386, 66387, 66388, 66389 were selected from the ORFs having the nucleotide sequence represented by SEQ ID NO: 5436.

The probes having the nucleotide sequence represented by SEQ ID NOS: 66390, 66391, 66392, 66393, 66394, 66395, 66396, 66397, 66398, 66399, 66400 were selected from the ORFs having the nucleotide sequence represented by SEQ ID NO: 5437.

The probes having the nucleotide sequence represented by SEQ ID NOS: 66401, 66402, 66403, 66404, 66405, 66406, 66407, 66408, 66409, 66410, 66411 were selected from the ORFs having the nucleotide sequence represented by SEQ ID NO: 5438.

The probes having the nucleotide sequence represented by SEQ ID NOS: 66412, 66413, 66414, 66415, 66416, 66417, 66418, 66419, 66420, 66421, 66422 were selected from the ORFs having the nucleotide sequence represented by SEQ ID NO: 5439.

The probes having the nucleotide sequence represented by SEQ ID NOS: 66423, 66424, 66425, 66426, 66427, 66428, 66429, 66430, 66431, 66432, 66433 were selected from the ORFs having the nucleotide sequence represented by SEQ ID NO: 5440.

The probes having the nucleotide sequence represented by SEQ ID NOS: 66434, 66435, 66436, 66437, 66438, 66439, 66440, 66441, 66442, 66443, 66444 were selected from the ORFs having the nucleotide sequence represented by SEQ ID NO: 5441.

The probes having the nucleotide sequence represented by SEQ ID NOS: 66445, 66446, 66447, 66448, 66449, 66450, 66451, 66452, 66453, 66454, 66455 were selected from the ORFs having the nucleotide sequence represented by SEQ ID NO: 5442.

The probes having the nucleotide sequence represented by SEQ ID NOS: 66456, 66457, 66458, 66459, 66460, 66461, 66462, 66463, 66464, 66465, 66466 were selected from the ORFs having the nucleotide sequence represented by SEQ ID NO: 5443.

The probes having the nucleotide sequence represented by SEQ ID NOS: 66467, 66468, 66469, 66470, 66471, 66472, 66473, 66474, 66475, 66476, 66477 were selected from the ORFs having the nucleotide sequence represented by SEQ ID NO: 5444.

The probes having the nucleotide sequence represented by SEQ ID NOS: 66478, 66479, 66480, 66481, 66482, 66483, 66484, 66485, 66486, 66487, 66488 were selected from the ORFs having the nucleotide sequence represented by SEQ ID NO: 5445.

The probes having the nucleotide sequence represented by SEQ ID NOS: 66489, 66490, 66491, 66492, 66493, 66494, 66495, 66496, 66497, 66498, 66499 were selected from the ORFs having the nucleotide sequence represented by SEQ ID NO: 5446.

The probes having the nucleotide sequence represented by SEQ ID NOS: 66500, 66501, 66502, 66503, 66504, 66505, 66506, 66507, 66508, 66509, 66510 were selected from the ORFs having the nucleotide sequence represented by SEQ ID NO: 5447.

The probes having the nucleotide sequence represented by SEQ ID NOS: 66511, 66512, 66513, 66514, 66515, 66516, 66517, 66518, 66519, 66520, 66521 were selected from the ORFs having the nucleotide sequence represented by SEQ ID NO: 5448.

The probes having the nucleotide sequence represented by SEQ ID NOS: 66522, 66523, 66524, 66525, 66526, 66527, 66528, 66529, 66530, 66531, 66532 were selected from the ORFs having the nucleotide sequence represented by SEQ ID NO: 5449.

The probes having the nucleotide sequence represented by SEQ ID NOS: 66533, 66534, 66535, 66536, 66537, 66538, 66539, 66540, 66541, 66542, 66543 were selected from the ORFs having the nucleotide sequence represented by SEQ ID NO: 5450.

The probes having the nucleotide sequence represented by SEQ ID NOS: 66544, 66545, 66546, 66547, 66548, 66549, 66550, 66551, 66552, 66553, 66554 were selected from the ORFs having the nucleotide sequence represented by SEQ ID NO: 5451.

The probes having the nucleotide sequence represented by SEQ ID NOS: 66555, 66556, 66557, 66558, 66559, 66560, 66561, 66562, 66563, 66564, 66565 were selected from the ORFs having the nucleotide sequence represented by SEQ ID NO: 5452.

The probes having the nucleotide sequence represented by SEQ ID NOS: 66566, 66567, 66568, 66569, 66570, 66571, 66572, 66573, 66574, 66575, 66576 were selected from the ORFs having the nucleotide sequence represented by SEQ ID NO: 5453.

The probes having the nucleotide sequence represented by SEQ ID NOS: 66577, 66578, 66579, 66580, 66581, 66582, 66583, 66584, 66585, 66586, 66587 were selected from the ORFs having the nucleotide sequence represented by SEQ ID NO: 5454.

The probes having the nucleotide sequence represented by SEQ ID NOS: 66588, 66589, 66590, 66591, 66592, 66593, 66594, 66595, 66596, 66597, 66598 were selected from the ORFs having the nucleotide sequence represented by SEQ ID NO: 5455.

The probes having the nucleotide sequence represented by SEQ ID NOS: 66599, 66600, 66601, 66602, 66603, 66604, 66605, 66606, 66607, 66608, 66609 were selected from the ORFs having the nucleotide sequence represented by SEQ ID NO: 5456.

The probes having the nucleotide sequence represented by SEQ ID NOS: 66610, 66611, 66612, 66613, 66614, 66615, 66616, 66617, 66618, 66619, 66620 were selected from the ORFs having the nucleotide sequence represented by SEQ ID NO: 5457.

The probes having the nucleotide sequence represented by SEQ ID NOS: 66621, 66622, 66623, 66624, 66625, 66626, 66627, 66628, 66629, 66630, 66631 were selected from the ORFs having the nucleotide sequence represented by SEQ ID NO: 5458.

The probes having the nucleotide sequence represented by SEQ ID NOS: 66632, 66633, 66634, 66635, 66636, 66637, 66638, 66639, 66640, 66641, 66642 were selected from the ORFs having the nucleotide sequence represented by SEQ ID NO: 5459.

The probes having the nucleotide sequence represented by SEQ ID NOS: 66643, 66644, 66645, 66646, 66647, 66648, 66649, 66650, 66651, 66652, 66653 were selected from the ORFs having the nucleotide sequence represented by SEQ ID NO: 5460.

The probes having the nucleotide sequence represented by SEQ ID NOS: 66654, 66655, 66656, 66657, 66658, 66659, 66660, 66661, 66662, 66663, 66664 were selected from the ORFs having the nucleotide sequence represented by SEQ ID NO: 5461.

The probes having the nucleotide sequence represented by SEQ ID NOS: 66665, 66666, 66667, 66668, 66669, 66670, 66671, 66672, 66673, 66674, 66675 were selected from the ORFs having the nucleotide sequence represented by SEQ ID NO: 5462.

The probes having the nucleotide sequence represented by SEQ ID NOS: 66676, 66677, 66678, 66679, 66680, 66681, 66682, 66683, 66684, 66685, 66686 were selected from the ORFs having the nucleotide sequence represented by SEQ ID NO: 5463.

The probes having the nucleotide sequence represented by SEQ ID NOS: 66687, 66688, 66689, 66690, 66691, 66692, 66693, 66694, 66695, 66696, 66697 were selected from the ORFs having the nucleotide sequence represented by SEQ ID NO: 5464.

The probes having the nucleotide sequence represented by SEQ ID NOS: 66698, 66699, 66700, 66701, 66702, 66703, 66704, 66705, 66706, 66707, 66708 were selected from the ORFs having the nucleotide sequence represented by SEQ ID NO: 5465.

The probes having the nucleotide sequence represented by SEQ ID NOS: 66709, 66710, 66711, 66712, 66713, 66714, 66715, 66716, 66717, 66718, 66719 were selected from the ORFs having the nucleotide sequence represented by SEQ ID NO: 5466.

The probes having the nucleotide sequence represented by SEQ ID NOS: 66720, 66721, 66722, 66723, 66724, 66725, 66726, 66727, 66728, 66729, 66730 were selected from the ORFs having the nucleotide sequence represented by SEQ ID NO: 5467.

The probes having the nucleotide sequence represented by SEQ ID NOS: 66731, 66732, 66733, 66734, 66735, 66736, 66737, 66738, 66739, 66740, 66741 were selected from the ORFs having the nucleotide sequence represented by SEQ ID NO: 5468.

The probes having the nucleotide sequence represented by SEQ ID NOS: 66742, 66743, 66744, 66745, 66746, 66747, 66748, 66749, 66750, 66751, 66752 were selected from the ORFs having the nucleotide sequence represented by SEQ ID NO: 5469.

The probes having the nucleotide sequence represented by SEQ ID NOS: 66753, 66754, 66755, 66756, 66757, 66758, 66759, 66760, 66761, 66762, 66763 were selected from the ORFs having the nucleotide sequence represented by SEQ ID NO: 5470.

The probes having the nucleotide sequence represented by SEQ ID NOS: 66764, 66765, 66766, 66767, 66768, 66769, 66770, 66771, 66772, 66773, 66774 were selected from the ORFs having the nucleotide sequence represented by SEQ ID NO: 5471.

The probes having the nucleotide sequence represented by SEQ ID NOS: 66775, 66776, 66777, 66778, 66779, 66780, 66781, 66782, 66783, 66784, 66785 were selected from the ORFs having the nucleotide sequence represented by SEQ ID NO: 5472.

The probes having the nucleotide sequence represented by SEQ ID NOS: 66786, 66787, 66788, 66789, 66790, 66791, 66792, 66793, 66794, 66795, 66796 were selected from the ORFs having the nucleotide sequence represented by SEQ ID NO: 5473.

The probes having the nucleotide sequence represented by SEQ ID NOS: 66797, 66798, 66799, 66800, 66801, 66802, 66803, 66804, 66805, 66806, 66807 were selected from the ORFs having the nucleotide sequence represented by SEQ ID NO: 5474.

The probes having the nucleotide sequence represented by SEQ ID NOS: 66808, 66809, 66810, 66811, 66812, 66813, 66814, 66815, 66816, 66817, 66818 were selected from the ORFs having the nucleotide sequence represented by SEQ ID NO: 5475.

The probes having the nucleotide sequence represented by SEQ ID NOS: 66819, 66820, 66821, 66822, 66823, 66824, 66825, 66826, 66827, 66828, 66829 were selected from the ORFs having the nucleotide sequence represented by SEQ ID NO: 5476.

The probes having the nucleotide sequence represented by SEQ ID NOS: 66830, 66831, 66832, 66833, 66834, 66835, 66836, 66837, 66838, 66839, 66840 were selected from the ORFs having the nucleotide sequence represented by SEQ ID NO: 5477.

The probes having the nucleotide sequence represented by SEQ ID NOS: 66841, 66842, 66843, 66844, 66845, 66846, 66847, 66848, 66849, 66850, 66851 were selected from the ORFs having the nucleotide sequence represented by SEQ ID NO: 5478.

The probes having the nucleotide sequence represented by SEQ ID NOS: 66852, 66853, 66854, 66855, 66856, 66857, 66858, 66859, 66860, 66861, 66862 were selected from the ORFs having the nucleotide sequence represented by SEQ ID NO: 5479.

The probes having the nucleotide sequence represented by SEQ ID NOS: 66863, 66864, 66865, 66866, 66867, 66868, 66869, 66870, 66871, 66872, 66873 were selected from the ORFs having the nucleotide sequence represented by SEQ ID NO: 5480.

The probes having the nucleotide sequence represented by SEQ ID NOS: 66874, 66875, 66876, 66877, 66878, 66879, 66880, 66881, 66882, 66883, 66884 were selected from the ORFs having the nucleotide sequence represented by SEQ ID NO: 5481.

The probes having the nucleotide sequence represented by SEQ ID NOS: 66885, 66886, 66887, 66888, 66889, 66890, 66891, 66892, 66893, 66894, 66895 were selected from the ORFs having the nucleotide sequence represented by SEQ ID NO: 5482.

The probes having the nucleotide sequence represented by SEQ ID NOS: 66896, 66897, 66898, 66899, 66900, 66901, 66902, 66903, 66904, 66905, 66906 were selected from the ORFs having the nucleotide sequence represented by SEQ ID NO: 5483.

The probes having the nucleotide sequence represented by SEQ ID NOS: 66907, 66908, 66909, 66910, 66911, 66912, 66913, 66914, 66915, 66916, 66917 were selected from the ORFs having the nucleotide sequence represented by SEQ ID NO: 5484.

The probes having the nucleotide sequence represented by SEQ ID NOS: 66918, 66919, 66920, 66921, 66922, 66923, 66924, 66925, 66926, 66927, 66928 were selected from the ORFs having the nucleotide sequence represented by SEQ ID NO: 5485.

The probes having the nucleotide sequence represented by SEQ ID NOS: 66929, 66930, 66931, 66932, 66933, 66934, 66935, 66936, 66937, 66938, 66939 were selected from the ORFs having the nucleotide sequence represented by SEQ ID NO: 5486.

The probes having the nucleotide sequence represented by SEQ ID NOS: 66940, 66941, 66942, 66943, 66944, 66945, 66946, 66947, 66948, 66949, 66950 were selected from the ORFs having the nucleotide sequence represented by SEQ ID NO: 5487.

The probes having the nucleotide sequence represented by SEQ ID NOS: 66951, 66952, 66953, 66954, 66955, 66956, 66957, 66958, 66959, 66960, 66961 were selected from the ORFs having the nucleotide sequence represented by SEQ ID NO: 5488.

The probes having the nucleotide sequence represented by SEQ ID NOS: 66962, 66963, 66964, 66965, 66966, 66967, 66968, 66969, 66970, 66971, 66972 were selected from the ORFs having the nucleotide sequence represented by SEQ ID NO: 5489.

The probes having the nucleotide sequence represented by SEQ ID NOS: 66973, 66974, 66975, 66976, 66977, 66978, 66979, 66980, 66981, 66982, 66983 were selected from the ORFs having the nucleotide sequence represented by SEQ ID NO: 5490.

The probes having the nucleotide sequence represented by SEQ ID NOS: 66984, 66985, 66986, 66987, 66988, 66989, 66990, 66991, 66992, 66993, 66994 were selected from the ORFs having the nucleotide sequence represented by SEQ ID NO: 5491.

The probes having the nucleotide sequence represented by SEQ ID NOS: 66995, 66996, 66997, 66998, 66999, 67000, 67001, 67002, 67003, 67004, 67005 were selected from the ORFs having the nucleotide sequence represented by SEQ ID NO: 5492.

The probes having the nucleotide sequence represented by SEQ ID NOS: 67006, 67007, 67008, 67009, 67010, 67011, 67012, 67013, 67014, 67015, 67016 were selected from the ORFs having the nucleotide sequence represented by SEQ ID NO: 5493.

The probes having the nucleotide sequence represented by SEQ ID NOS: 67017, 67018, 67019, 67020, 67021, 67022, 67023, 67024, 67025, 67026, 67027 were selected from the ORFs having the nucleotide sequence represented by SEQ ID NO: 5494.

The probes having the nucleotide sequence represented by SEQ ID NOS: 67028, 67029, 67030, 67031, 67032, 67033, 67034, 67035, 67036, 67037, 67038 were selected from the ORFs having the nucleotide sequence represented by SEQ ID NO: 5495.

The probes having the nucleotide sequence represented by SEQ ID NOS: 67039, 67040, 67041, 67042, 67043, 67044, 67045, 67046, 67047, 67048, 67049 were selected from the ORFs having the nucleotide sequence represented by SEQ ID NO: 5496.

The probes having the nucleotide sequence represented by SEQ ID NOS: 67050, 67051, 67052, 67053, 67054, 67055, 67056, 67057, 67058, 67059, 67060 were selected from the ORFs having the nucleotide sequence represented by SEQ ID NO: 5497.

The probes having the nucleotide sequence represented by SEQ ID NOS: 67061, 67062, 67063, 67064, 67065, 67066, 67067, 67068, 67069, 67070, 67071 were selected from the ORFs having the nucleotide sequence represented by SEQ ID NO: 5498.

The probes having the nucleotide sequence represented by SEQ ID NOS: 67072, 67073, 67074, 67075, 67076, 67077, 67078, 67079, 67080, 67081, 67082 were selected from the ORFs having the nucleotide sequence represented by SEQ ID NO: 5499.

The probes having the nucleotide sequence represented by SEQ ID NOS: 67083, 67084, 67085, 67086, 67087, 67088, 67089, 67090, 67091, 67092, 67093 were selected from the ORFs having the nucleotide sequence represented by SEQ ID NO: 5500.

The probes having the nucleotide sequence represented by SEQ ID NOS: 67094, 67095, 67096, 67097, 67098, 67099, 67100, 67101, 67102, 67103, 67104 were selected from the ORFs having the nucleotide sequence represented by SEQ ID NO: 5501.

The probes having the nucleotide sequence represented by SEQ ID NOS: 67105, 67106, 67107, 67108, 67109, 67110, 67111, 67112, 67113, 67114, 67115 were selected from the ORFs having the nucleotide sequence represented by SEQ ID NO: 5502.

The probes having the nucleotide sequence represented by SEQ ID NOS: 67116, 67117, 67118, 67119, 67120, 67121, 67122, 67123, 67124, 67125, 67126 were selected from the ORFs having the nucleotide sequence represented by SEQ ID NO: 5503.

The probes having the nucleotide sequence represented by SEQ ID NOS: 67127, 67128, 67129, 67130, 67131, 67132, 67133, 67134, 67135, 67136, 67137 were selected from the ORFs having the nucleotide sequence represented by SEQ ID NO: 5504.

The probes having the nucleotide sequence represented by SEQ ID NOS: 67138, 67139, 67140, 67141, 67142, 67143, 67144, 67145, 67146, 67147, 67148 were selected from the ORFs having the nucleotide sequence represented by SEQ ID NO: 5505.

The probes having the nucleotide sequence represented by SEQ ID NOS: 67149, 67150, 67151, 67152, 67153, 67154, 67155, 67156, 67157, 67158, 67159 were selected from the ORFs having the nucleotide sequence represented by SEQ ID NO: 5506.

The probes having the nucleotide sequence represented by SEQ ID NOS: 67160, 67161, 67162, 67163, 67164, 67165, 67166, 67167, 67168, 67169, 67170 were selected from the ORFs having the nucleotide sequence represented by SEQ ID NO: 5507.

The probes having the nucleotide sequence represented by SEQ ID NOS: 67171, 67172, 67173, 67174, 67175, 67176, 67177, 67178, 67179, 67180, 67181 were selected from the ORFs having the nucleotide sequence represented by SEQ ID NO: 5508.

The probes having the nucleotide sequence represented by SEQ ID NOS: 67182, 67183, 67184, 67185, 67186, 67187, 67188, 67189, 67190, 67191, 67192 were selected from the ORFs having the nucleotide sequence represented by SEQ ID NO: 5509.

The probes having the nucleotide sequence represented by SEQ ID NOS: 67193, 67194, 67195, 67196, 67197, 67198, 67199, 67200, 67201, 67202, 67203 were selected from the ORFs having the nucleotide sequence represented by SEQ ID NO: 5510.

The probes having the nucleotide sequence represented by SEQ ID NOS: 67204, 67205, 67206, 67207, 67208, 67209, 67210, 67211, 67212, 67213, 67214 were selected from the ORFs having the nucleotide sequence represented by SEQ ID NO: 5511.

The probes having the nucleotide sequence represented by SEQ ID NOS: 67215, 67216, 67217, 67218, 67219, 67220, 67221, 67222, 67223, 67224, 67225 were selected from the ORFs having the nucleotide sequence represented by SEQ ID NO: 5512.

The probes having the nucleotide sequence represented by SEQ ID NOS: 67226, 67227, 67228, 67229, 67230, 67231, 67232, 67233, 67234, 67235, 67236 were selected from the ORFs having the nucleotide sequence represented by SEQ ID NO: 5513.

The probes having the nucleotide sequence represented by SEQ ID NOS: 67237, 67238, 67239, 67240, 67241, 67242, 67243, 67244, 67245, 67246, 67247 were selected from the ORFs having the nucleotide sequence represented by SEQ ID NO: 5514.

The probes having the nucleotide sequence represented by SEQ ID NOS: 67248, 67249, 67250, 67251, 67252, 67253, 67254, 67255, 67256, 67257, 67258 were selected from the ORFs having the nucleotide sequence represented by SEQ ID NO: 5515.

The probes having the nucleotide sequence represented by SEQ ID NOS: 67259, 67260, 67261, 67262, 67263, 67264, 67265, 67266, 67267, 67268, 67269 were selected from the ORFs having the nucleotide sequence represented by SEQ ID NO: 5516.

The probes having the nucleotide sequence represented by SEQ ID NOS: 67270, 67271, 67272, 67273, 67274, 67275, 67276, 67277, 67278, 67279, 67280 were selected from the ORFs having the nucleotide sequence represented by SEQ ID NO: 5517.

The probes having the nucleotide sequence represented by SEQ ID NOS: 67281, 67282, 67283, 67284, 67285, 67286, 67287, 67288, 67289, 67290, 67291 were selected from the ORFs having the nucleotide sequence represented by SEQ ID NO: 5518.

The probes having the nucleotide sequence represented by SEQ ID NOS: 67292, 67293, 67294, 67295, 67296, 67297, 67298, 67299, 67300, 67301, 67302 were selected from the ORFs having the nucleotide sequence represented by SEQ ID NO: 5519.

The probes having the nucleotide sequence represented by SEQ ID NOS: 67303, 67304, 67305, 67306, 67307, 67308, 67309, 67310, 67311, 67312, 67313 were selected from the ORFs having the nucleotide sequence represented by SEQ ID NO: 5520.

The probes having the nucleotide sequence represented by SEQ ID NOS: 67314, 67315, 67316, 67317, 67318, 67319, 67320, 67321, 67322, 67323, 67324 were selected from the ORFs having the nucleotide sequence represented by SEQ ID NO: 5521.

The probes having the nucleotide sequence represented by SEQ ID NOS: 67325, 67326, 67327, 67328, 67329, 67330, 67331, 67332, 67333, 67334, 67335 were selected from the ORFs having the nucleotide sequence represented by SEQ ID NO: 5522.

The probes having the nucleotide sequence represented by SEQ ID NOS: 67336, 67337, 67338, 67339, 67340, 67341, 67342, 67343, 67344, 67345, 67346 were selected from the ORFs having the nucleotide sequence represented by SEQ ID NO: 5523.

The probes having the nucleotide sequence represented by SEQ ID NOS: 67347, 67348, 67349, 67350, 67351, 67352, 67353, 67354, 67355, 67356, 67357, 67358, 67359, 67360, 67361, 67362, 67363, 67364, 67365, 67366, 67367, 67368 were selected from the ORFs having the nucleotide sequence represented by SEQ ID NO: 5524.

The probes having the nucleotide sequence represented by SEQ ID NOS: 67369, 67370, 67371, 67372, 67373, 67374, 67375, 67376, 67377, 67378, 67379 were selected from the ORFs having the nucleotide sequence represented by SEQ ID NO: 5525.

The probes having the nucleotide sequence represented by SEQ ID NOS: 67380, 67381, 67382, 67383, 67384, 67385, 67386, 67387, 67388, 67389, 67390 were selected from the ORFs having the nucleotide sequence represented by SEQ ID NO: 5526.

The probes having the nucleotide sequence represented by SEQ ID NOS: 67391, 67392, 67393, 67394, 67395, 67396, 67397, 67398, 67399, 67400, 67401 were selected from the ORFs having the nucleotide sequence represented by SEQ ID NO: 5527.

The probes having the nucleotide sequence represented by SEQ ID NOS: 67402, 67403, 67404, 67405, 67406, 67407, 67408, 67409, 67410, 67411, 67412 were selected from the ORFs having the nucleotide sequence represented by SEQ ID NO: 5528.

The probes having the nucleotide sequence represented by SEQ ID NOS: 67413, 67414, 67415, 67416, 67417, 67418, 67419, 67420, 67421, 67422, 67423 were selected from the ORFs having the nucleotide sequence represented by SEQ ID NO: 5529.

The probes having the nucleotide sequence represented by SEQ ID NOS: 67424, 67425, 67426, 67427, 67428, 67429, 67430, 67431, 67432, 67433, 67434 were selected from the ORFs having the nucleotide sequence represented by SEQ ID NO: 5530.

The probes having the nucleotide sequence represented by SEQ ID NOS: 67435, 67436, 67437, 67438, 67439, 67440, 67441, 67442, 67443, 67444, 67445 were selected from the ORFs having the nucleotide sequence represented by SEQ ID NO: 5531.

The probes having the nucleotide sequence represented by SEQ ID NOS: 67446, 67447, 67448, 67449, 67450, 67451, 67452, 67453, 67454, 67455, 67456, 67457, 67458, 67459, 67460, 67461, 67462, 67463, 67464, 67465, 67466, 67467 were selected from the ORFs having the nucleotide sequence represented by SEQ ID NO: 5532.

The probes having the nucleotide sequence represented by SEQ ID NOS: 67468, 67469, 67470, 67471, 67472, 67473, 67474, 67475, 67476, 67477, 67478 were selected from the ORFs having the nucleotide sequence represented by SEQ ID NO: 5533.

The probes having the nucleotide sequence represented by SEQ ID NOS: 67479, 67480, 67481, 67482, 67483, 67484, 67485, 67486, 67487, 67488, 67489 were selected from the ORFs having the nucleotide sequence represented by SEQ ID NO: 5534.

The probes having the nucleotide sequence represented by SEQ ID NOS: 67490, 67491, 67492, 67493, 67494, 67495, 67496, 67497, 67498, 67499, 67500 were selected from the ORFs having the nucleotide sequence represented by SEQ ID NO: 5535.

The probes having the nucleotide sequence represented by SEQ ID NOS: 67501, 67502, 67503, 67504, 67505, 67506, 67507, 67508, 67509, 67510, 67511 were selected from the ORFs having the nucleotide sequence represented by SEQ ID NO: 5536.

The probes having the nucleotide sequence represented by SEQ ID NOS: 67512, 67513, 67514, 67515, 67516, 67517, 67518, 67519, 67520, 67521, 67522 were selected from the ORFs having the nucleotide sequence represented by SEQ ID NO: 5537.

The probes having the nucleotide sequence represented by SEQ ID NOS: 67523, 67524, 67525, 67526, 67527, 67528, 67529, 67530, 67531, 67532, 67533 were selected from the ORFs having the nucleotide sequence represented by SEQ ID NO: 5538.

The probes having the nucleotide sequence represented by SEQ ID NOS: 67534, 67535, 67536, 67537, 67538, 67539, 67540, 67541, 67542, 67543, 67544 were selected from the ORFs having the nucleotide sequence represented by SEQ ID NO: 5539.

The probes having the nucleotide sequence represented by SEQ ID NOS: 67545, 67546, 67547, 67548, 67549, 67550, 67551, 67552, 67553, 67554, 67555 were selected from the ORFs having the nucleotide sequence represented by SEQ ID NO: 5540.

The probes having the nucleotide sequence represented by SEQ ID NOS: 67556, 67557, 67558, 67559, 67560, 67561, 67562, 67563, 67564, 67565, 67566 were selected from the ORFs having the nucleotide sequence represented by SEQ ID NO: 5541.

The probes having the nucleotide sequence represented by SEQ ID NOS: 67567, 67568, 67569, 67570, 67571, 67572, 67573, 67574, 67575, 67576, 67577 were selected from the ORFs having the nucleotide sequence represented by SEQ ID NO: 5542.

The probes having the nucleotide sequence represented by SEQ ID NOS: 67578, 67579, 67580, 67581, 67582, 67583, 67584, 67585, 67586, 67587, 67588 were selected from the ORFs having the nucleotide sequence represented by SEQ ID NO: 5543.

The probes having the nucleotide sequence represented by SEQ ID NOS: 67589, 67590, 67591, 67592, 67593, 67594, 67595, 67596, 67597, 67598, 67599 were selected from the ORFs having the nucleotide sequence represented by SEQ ID NO: 5544.

The probes having the nucleotide sequence represented by SEQ ID NOS: 67600, 67601, 67602, 67603, 67604, 67605, 67606, 67607, 67608, 67609, 67610 were selected from the ORFs having the nucleotide sequence represented by SEQ ID NO: 5545.

The probes having the nucleotide sequence represented by SEQ ID NOS: 67611, 67612, 67613, 67614, 67615, 67616, 67617, 67618, 67619, 67620, 67621 were selected from the ORFs having the nucleotide sequence represented by SEQ ID NO: 5546.

The probes having the nucleotide sequence represented by SEQ ID NOS: 67622, 67623, 67624, 67625, 67626, 67627, 67628, 67629, 67630, 67631, 67632 were selected from the ORFs having the nucleotide sequence represented by SEQ ID NO: 5547.

The probes having the nucleotide sequence represented by SEQ ID NOS: 67633, 67634, 67635, 67636, 67637, 67638, 67639, 67640, 67641, 67642, 67643 were selected from the ORFs having the nucleotide sequence represented by SEQ ID NO: 5548.

The probes having the nucleotide sequence represented by SEQ ID NOS: 67644, 67645, 67646, 67647, 67648, 67649, 67650, 67651, 67652, 67653, 67654 were selected from the ORFs having the nucleotide sequence represented by SEQ ID NO: 5549.

The probes having the nucleotide sequence represented by SEQ ID NOS: 67655, 67656, 67657, 67658, 67659, 67660, 67661, 67662, 67663, 67664, 67665 were selected from the ORFs having the nucleotide sequence represented by SEQ ID NO: 5550.

The probes having the nucleotide sequence represented by SEQ ID NOS: 67666, 67667, 67668, 67669, 67670, 67671, 67672, 67673, 67674, 67675, 67676, 67677, 67678, 67679, 67680, 67681, 67682, 67683, 67684, 67685, 67686, 67687 were selected from the ORFs having the nucleotide sequence represented by SEQ ID NO: 5551.

The probes having the nucleotide sequence represented by SEQ ID NOS: 67688, 67689, 67690, 67691, 67692, 67693, 67694, 67695, 67696, 67697, 67698 were selected from the ORFs having the nucleotide sequence represented by SEQ ID NO: 5552.

The probes having the nucleotide sequence represented by SEQ ID NOS: 67699, 67700, 67701, 67702, 67703, 67704, 67705, 67706, 67707, 67708, 67709 were selected from the ORFs having the nucleotide sequence represented by SEQ ID NO: 5553.

The probes having the nucleotide sequence represented by SEQ ID NOS: 67710, 67711, 67712, 67713, 67714, 67715, 67716, 67717, 67718, 67719, 67720 were selected from the ORFs having the nucleotide sequence represented by SEQ ID NO: 5554.

The probes having the nucleotide sequence represented by SEQ ID NOS: 67721, 67722, 67723, 67724, 67725, 67726, 67727, 67728, 67729, 67730, 67731 were selected from the ORFs having the nucleotide sequence represented by SEQ ID NO: 5555.

The probes having the nucleotide sequence represented by SEQ ID NOS: 67732, 67733, 67734, 67735, 67736, 67737, 67738, 67739, 67740, 67741, 67742 were selected from the ORFs having the nucleotide sequence represented by SEQ ID NO: 5556.

The probes having the nucleotide sequence represented by SEQ ID NOS: 67743, 67744, 67745, 67746, 67747, 67748, 67749, 67750, 67751, 67752, 67753 were selected from the ORFs having the nucleotide sequence represented by SEQ ID NO: 5557.

The probes having the nucleotide sequence represented by SEQ ID NOS: 67754, 67755, 67756, 67757, 67758, 67759, 67760, 67761, 67762, 67763, 67764 were selected from the ORFs having the nucleotide sequence represented by SEQ ID NO: 5558.

The probes having the nucleotide sequence represented by SEQ ID NOS: 67765, 67766, 67767, 67768, 67769, 67770, 67771, 67772, 67773, 67774, 67775 were selected from the ORFs having the nucleotide sequence represented by SEQ ID NO: 5559.

The probes having the nucleotide sequence represented by SEQ ID NOS: 67776, 67777, 67778, 67779, 67780, 67781, 67782, 67783, 67784, 67785, 67786 were selected from the ORFs having the nucleotide sequence represented by SEQ ID NO: 5560.

The probes having the nucleotide sequence represented by SEQ ID NOS: 67787, 67788, 67789, 67790, 67791, 67792, 67793, 67794, 67795, 67796, 67797 were selected from the ORFs having the nucleotide sequence represented by SEQ ID NO: 5561.

The probes having the nucleotide sequence represented by SEQ ID NOS: 67798, 67799, 67800, 67801, 67802, 67803, 67804, 67805, 67806, 67807, 67808 were selected from the ORFs having the nucleotide sequence represented by SEQ ID NO: 5562.

The probes having the nucleotide sequence represented by SEQ ID NOS: 67809, 67810, 67811, 67812, 67813, 67814, 67815, 67816, 67817, 67818, 67819 were selected from the ORFs having the nucleotide sequence represented by SEQ ID NO: 5563.

The probes having the nucleotide sequence represented by SEQ ID NOS: 67820, 67821, 67822, 67823, 67824, 67825, 67826, 67827, 67828, 67829, 67830 were selected from the ORFs having the nucleotide sequence represented by SEQ ID NO: 5564.

The probes having the nucleotide sequence represented by SEQ ID NOS: 67831, 67832, 67833, 67834, 67835, 67836, 67837, 67838, 67839, 67840, 67841 were selected from the ORFs having the nucleotide sequence represented by SEQ ID NO: 5565.

The probes having the nucleotide sequence represented by SEQ ID NOS: 67842, 67843, 67844, 67845, 67846, 67847, 67848, 67849, 67850, 67851, 67852 were selected from the ORFs having the nucleotide sequence represented by SEQ ID NO: 5566.

The probes having the nucleotide sequence represented by SEQ ID NOS: 67853, 67854, 67855, 67856, 67857, 67858, 67859, 67860, 67861, 67862, 67863 were selected from the ORFs having the nucleotide sequence represented by SEQ ID NO: 5567.

The probes having the nucleotide sequence represented by SEQ ID NOS: 67864, 67865, 67866, 67867, 67868, 67869, 67870, 67871, 67872, 67873, 67874 were selected from the ORFs having the nucleotide sequence represented by SEQ ID NO: 5568.

The probes having the nucleotide sequence represented by SEQ ID NOS: 67875, 67876, 67877, 67878, 67879, 67880, 67881, 67882, 67883, 67884, 67885 were selected from the ORFs having the nucleotide sequence represented by SEQ ID NO: 5569.

The probes having the nucleotide sequence represented by SEQ ID NOS: 67886, 67887, 67888, 67889, 67890, 67891, 67892, 67893, 67894, 67895, 67896 were selected from the ORFs having the nucleotide sequence represented by SEQ ID NO: 5570.

The probes having the nucleotide sequence represented by SEQ ID NOS: 67897, 67898, 67899, 67900, 67901, 67902, 67903, 67904, 67905, 67906, 67907 were selected from the ORFs having the nucleotide sequence represented by SEQ ID NO: 5571.

The probes having the nucleotide sequence represented by SEQ ID NOS: 67908, 67909, 67910, 67911, 67912, 67913, 67914, 67915, 67916, 67917, 67918 were selected from the ORFs having the nucleotide sequence represented by SEQ ID NO: 5572.

The probes having the nucleotide sequence represented by SEQ ID NOS: 67919, 67920, 67921, 67922, 67923, 67924, 67925, 67926, 67927, 67928, 67929 were selected from the ORFs having the nucleotide sequence represented by SEQ ID NO: 5573.

The probes having the nucleotide sequence represented by SEQ ID NOS: 67930, 67931, 67932, 67933, 67934, 67935, 67936, 67937, 67938, 67939, 67940 were selected from the ORFs having the nucleotide sequence represented by SEQ ID NO: 5574.

The probes having the nucleotide sequence represented by SEQ ID NOS: 67941, 67942, 67943, 67944, 67945, 67946, 67947, 67948, 67949, 67950, 67951 were selected from the ORFs having the nucleotide sequence represented by SEQ ID NO: 5575.

The probes having the nucleotide sequence represented by SEQ ID NOS: 67952, 67953, 67954, 67955, 67956, 67957, 67958, 67959, 67960, 67961, 67962 were selected from the ORFs having the nucleotide sequence represented by SEQ ID NO: 5576.

The probes having the nucleotide sequence represented by SEQ ID NOS: 67963, 67964, 67965, 67966, 67967, 67968, 67969, 67970, 67971, 67972, 67973 were selected from the ORFs having the nucleotide sequence represented by SEQ ID NO: 5577.

The probes having the nucleotide sequence represented by SEQ ID NOS: 67974, 67975, 67976, 67977, 67978, 67979, 67980, 67981, 67982, 67983, 67984 were selected from the ORFs having the nucleotide sequence represented by SEQ ID NO: 5578.

The probes having the nucleotide sequence represented by SEQ ID NOS: 67985, 67986, 67987, 67988, 67989, 67990, 67991, 67992, 67993, 67994, 67995 were selected from the ORFs having the nucleotide sequence represented by SEQ ID NO: 5579.

The probes having the nucleotide sequence represented by SEQ ID NOS: 67996, 67997, 67998, 67999, 68000, 68001, 68002, 68003, 68004, 68005, 68006 were selected from the ORFs having the nucleotide sequence represented by SEQ ID NO: 5580.

The probes having the nucleotide sequence represented by SEQ ID NOS: 68007, 68008, 68009, 68010, 68011, 68012, 68013, 68014, 68015, 68016, 68017 were selected from the ORFs having the nucleotide sequence represented by SEQ ID NO: 5581.

The probes having the nucleotide sequence represented by SEQ ID NOS: 68018, 68019, 68020, 68021, 68022, 68023, 68024, 68025, 68026, 68027, 68028 were selected from the ORFs having the nucleotide sequence represented by SEQ ID NO: 5582.

The probes having the nucleotide sequence represented by SEQ ID NOS: 68029, 68030, 68031, 68032, 68033, 68034, 68035, 68036, 68037, 68038, 68039 were selected from the ORFs having the nucleotide sequence represented by SEQ ID NO: 5583.

The probes having the nucleotide sequence represented by SEQ ID NOS: 68040, 68041, 68042, 68043, 68044, 68045, 68046, 68047, 68048, 68049, 68050 were selected from the ORFs having the nucleotide sequence represented by SEQ ID NO: 5584.

The probes having the nucleotide sequence represented by SEQ ID NOS: 68051, 68052, 68053, 68054, 68055, 68056, 68057, 68058, 68059, 68060, 68061 were selected from the ORFs having the nucleotide sequence represented by SEQ ID NO: 5585.

The probes having the nucleotide sequence represented by SEQ ID NOS: 68062, 68063, 68064, 68065, 68066, 68067, 68068, 68069, 68070, 68071, 68072 were selected from the ORFs having the nucleotide sequence represented by SEQ ID NO: 5586.

The probes having the nucleotide sequence represented by SEQ ID NOS: 68073, 68074, 68075, 68076, 68077, 68078, 68079, 68080, 68081, 68082, 68083 were selected from the ORFs having the nucleotide sequence represented by SEQ ID NO: 5587.

The probes having the nucleotide sequence represented by SEQ ID NOS: 68084, 68085, 68086, 68087, 68088, 68089, 68090, 68091, 68092, 68093, 68094 were selected from the ORFs having the nucleotide sequence represented by SEQ ID NO: 5588.

The probes having the nucleotide sequence represented by SEQ ID NOS: 68095, 68096, 68097, 68098, 68099, 68100, 68101, 68102, 68103, 68104, 68105 were selected from the ORFs having the nucleotide sequence represented by SEQ ID NO: 5589.

The probes having the nucleotide sequence represented by SEQ ID NOS: 68106, 68107, 68108, 68109, 68110, 68111, 68112, 68113, 68114, 68115, 68116 were selected from the ORFs having the nucleotide sequence represented by SEQ ID NO: 5590.

The probes having the nucleotide sequence represented by SEQ ID NOS: 68117, 68118, 68119, 68120, 68121, 68122, 68123, 68124, 68125, 68126, 68127 were selected from the ORFs having the nucleotide sequence represented by SEQ ID NO: 5591.

The probes having the nucleotide sequence represented by SEQ ID NOS: 68128, 68129, 68130, 68131, 68132, 68133, 68134, 68135, 68136, 68137, 68138 were selected from the ORFs having the nucleotide sequence represented by SEQ ID NO: 5592.

The probes having the nucleotide sequence represented by SEQ ID NOS: 68139, 68140, 68141, 68142, 68143, 68144, 68145, 68146, 68147, 68148, 68149 were selected from the ORFs having the nucleotide sequence represented by SEQ ID NO: 5593.

The probes having the nucleotide sequence represented by SEQ ID NOS: 68150, 68151, 68152, 68153, 68154, 68155, 68156, 68157, 68158, 68159, 68160 were selected from the ORFs having the nucleotide sequence represented by SEQ ID NO: 5594.

The probes having the nucleotide sequence represented by SEQ ID NOS: 68161, 68162, 68163, 68164, 68165, 68166, 68167, 68168, 68169, 68170, 68171 were selected from the ORFs having the nucleotide sequence represented by SEQ ID NO: 5595.

The probes having the nucleotide sequence represented by SEQ ID NOS: 68172, 68173, 68174, 68175, 68176, 68177, 68178, 68179, 68180, 68181, 68182 were selected from the ORFs having the nucleotide sequence represented by SEQ ID NO: 5596.

The probes having the nucleotide sequence represented by SEQ ID NOS: 68183, 68184, 68185, 68186, 68187, 68188, 68189, 68190, 68191, 68192, 68193 were selected from the ORFs having the nucleotide sequence represented by SEQ ID NO: 5597.

The probes having the nucleotide sequence represented by SEQ ID NOS: 68194, 68195, 68196, 68197, 68198, 68199, 68200, 68201, 68202, 68203, 68204 were selected from the ORFs having the nucleotide sequence represented by SEQ ID NO: 5598.

The probes having the nucleotide sequence represented by SEQ ID NOS: 68205, 68206, 68207, 68208, 68209, 68210, 68211, 68212, 68213, 68214, 68215 were selected from the ORFs having the nucleotide sequence represented by SEQ ID NO: 5599.

The probes having the nucleotide sequence represented by SEQ ID NOS: 68216, 68217, 68218, 68219, 68220, 68221, 68222, 68223, 68224, 68225, 68226 were selected from the ORFs having the nucleotide sequence represented by SEQ ID NO: 5600.

The probes having the nucleotide sequence represented by SEQ ID NOS: 68227, 68228, 68229, 68230, 68231, 68232, 68233, 68234, 68235, 68236, 68237 were selected from the ORFs having the nucleotide sequence represented by SEQ ID NO: 5601.

The probes having the nucleotide sequence represented by SEQ ID NOS: 68238, 68239, 68240, 68241, 68242, 68243, 68244, 68245, 68246, 68247, 68248 were selected from the ORFs having the nucleotide sequence represented by SEQ ID NO: 5602.

The probes having the nucleotide sequence represented by SEQ ID NOS: 68249, 68250, 68251, 68252, 68253, 68254, 68255, 68256, 68257, 68258, 68259 were selected from the ORFs having the nucleotide sequence represented by SEQ ID NO: 5603.

The probes having the nucleotide sequence represented by SEQ ID NOS: 68260, 68261, 68262, 68263, 68264, 68265, 68266, 68267, 68268, 68269, 68270 were selected from the ORFs having the nucleotide sequence represented by SEQ ID NO: 5604.

The probes having the nucleotide sequence represented by SEQ ID NOS: 68271, 68272, 68273, 68274, 68275, 68276, 68277, 68278, 68279, 68280, 68281 were selected from the ORFs having the nucleotide sequence represented by SEQ ID NO: 5605.

The probes having the nucleotide sequence represented by SEQ ID NOS: 68282, 68283, 68284, 68285, 68286, 68287, 68288, 68289, 68290, 68291, 68292 were selected from the ORFs having the nucleotide sequence represented by SEQ ID NO: 5606.

The probes having the nucleotide sequence represented by SEQ ID NOS: 68293, 68294, 68295, 68296, 68297, 68298, 68299, 68300, 68301, 68302, 68303 were selected from the ORFs having the nucleotide sequence represented by SEQ ID NO: 5607.

The probes having the nucleotide sequence represented by SEQ ID NOS: 68304, 68305, 68306, 68307, 68308, 68309, 68310, 68311, 68312, 68313, 68314 were selected from the ORFs having the nucleotide sequence represented by SEQ ID NO: 5608.

The probes having the nucleotide sequence represented by SEQ ID NOS: 68315, 68316, 68317, 68318, 68319, 68320, 68321, 68322, 68323, 68324, 68325 were selected from the ORFs having the nucleotide sequence represented by SEQ ID NO: 5609.

The probes having the nucleotide sequence represented by SEQ ID NOS: 68326, 68327, 68328, 68329, 68330, 68331, 68332, 68333, 68334, 68335, 68336 were selected from the ORFs having the nucleotide sequence represented by SEQ ID NO: 5610.

The probes having the nucleotide sequence represented by SEQ ID NOS: 68337, 68338, 68339, 68340, 68341, 68342, 68343, 68344, 68345, 68346, 68347 were selected from the ORFs having the nucleotide sequence represented by SEQ ID NO: 5611.

The probes having the nucleotide sequence represented by SEQ ID NOS: 68348, 68349, 68350, 68351, 68352, 68353, 68354, 68355, 68356, 68357, 68358 were selected from the ORFs having the nucleotide sequence represented by SEQ ID NO: 5612.

The probes having the nucleotide sequence represented by SEQ ID NOS: 68359, 68360, 68361, 68362, 68363, 68364, 68365, 68366, 68367, 68368, 68369 were selected from the ORFs having the nucleotide sequence represented by SEQ ID NO: 5613.

The probes having the nucleotide sequence represented by SEQ ID NOS: 68370, 68371, 68372, 68373, 68374, 68375, 68376, 68377, 68378, 68379, 68380 were selected from the ORFs having the nucleotide sequence represented by SEQ ID NO: 5614.

The probes having the nucleotide sequence represented by SEQ ID NOS: 68381, 68382, 68383, 68384, 68385, 68386, 68387, 68388, 68389, 68390, 68391 were selected from the ORFs having the nucleotide sequence represented by SEQ ID NO: 5615.

The probes having the nucleotide sequence represented by SEQ ID NOS: 68392, 68393, 68394, 68395, 68396, 68397, 68398, 68399, 68400, 68401, 68402 were selected from the ORFs having the nucleotide sequence represented by SEQ ID NO: 5616.

The probes having the nucleotide sequence represented by SEQ ID NOS: 68403, 68404, 68405, 68406, 68407, 68408, 68409, 68410, 68411, 68412, 68413 were selected from the ORFs having the nucleotide sequence represented by SEQ ID NO: 5617.

The probes having the nucleotide sequence represented by SEQ ID NOS: 68414, 68415, 68416, 68417, 68418, 68419, 68420, 68421, 68422, 68423, 68424 were selected from the ORFs having the nucleotide sequence represented by SEQ ID NO: 5618.

The probes having the nucleotide sequence represented by SEQ ID NOS: 68425, 68426, 68427, 68428, 68429, 68430, 68431, 68432, 68433, 68434, 68435 were selected from the ORFs having the nucleotide sequence represented by SEQ ID NO: 5619.

The probes having the nucleotide sequence represented by SEQ ID NOS: 68436, 68437, 68438, 68439, 68440, 68441, 68442, 68443, 68444, 68445, 68446 were selected from the ORFs having the nucleotide sequence represented by SEQ ID NO: 5620.

The probes having the nucleotide sequence represented by SEQ ID NOS: 68447, 68448, 68449, 68450, 68451, 68452, 68453, 68454, 68455, 68456, 68457 were selected from the ORFs having the nucleotide sequence represented by SEQ ID NO: 5621.

The probes having the nucleotide sequence represented by SEQ ID NOS: 68458, 68459, 68460, 68461, 68462, 68463, 68464, 68465, 68466, 68467, 68468 were selected from the ORFs having the nucleotide sequence represented by SEQ ID NO: 5622.

The probes having the nucleotide sequence represented by SEQ ID NOS: 68469, 68470, 68471, 68472, 68473, 68474, 68475, 68476, 68477, 68478, 68479 were selected from the ORFs having the nucleotide sequence represented by SEQ ID NO: 5623.

The probes having the nucleotide sequence represented by SEQ ID NOS: 68480, 68481, 68482, 68483, 68484, 68485, 68486, 68487, 68488, 68489, 68490 were selected from the ORFs having the nucleotide sequence represented by SEQ ID NO: 5624.

The probes having the nucleotide sequence represented by SEQ ID NOS: 68491, 68492, 68493, 68494, 68495, 68496, 68497, 68498, 68499, 68500, 68501 were selected from the ORFs having the nucleotide sequence represented by SEQ ID NO: 5625.

The probes having the nucleotide sequence represented by SEQ ID NOS: 68502, 68503, 68504, 68505, 68506, 68507, 68508, 68509, 68510, 68511, 68512 were selected from the ORFs having the nucleotide sequence represented by SEQ ID NO: 5626.

The probes having the nucleotide sequence represented by SEQ ID NOS: 68513, 68514, 68515, 68516, 68517, 68518, 68519, 68520, 68521, 68522, 68523 were selected from the ORFs having the nucleotide sequence represented by SEQ ID NO: 5627.

The probes having the nucleotide sequence represented by SEQ ID NOS: 68524, 68525, 68526, 68527, 68528, 68529, 68530, 68531, 68532, 68533, 68534 were selected from the ORFs having the nucleotide sequence represented by SEQ ID NO: 5628.

The probes having the nucleotide sequence represented by SEQ ID NOS: 68535, 68536, 68537, 68538, 68539, 68540, 68541, 68542, 68543, 68544, 68545 were selected from the ORFs having the nucleotide sequence represented by SEQ ID NO: 5629.

The probes having the nucleotide sequence represented by SEQ ID NOS: 68546, 68547, 68548, 68549, 68550, 68551, 68552, 68553, 68554, 68555, 68556 were selected from the ORFs having the nucleotide sequence represented by SEQ ID NO: 5630.

The probes having the nucleotide sequence represented by SEQ ID NOS: 68557, 68558, 68559, 68560, 68561, 68562, 68563, 68564, 68565, 68566, 68567 were selected from the ORFs having the nucleotide sequence represented by SEQ ID NO: 5631.

The probes having the nucleotide sequence represented by SEQ ID NOS: 68568, 68569, 68570, 68571, 68572, 68573, 68574, 68575, 68576, 68577, 68578 were selected from the ORFs having the nucleotide sequence represented by SEQ ID NO: 5632.

The probes having the nucleotide sequence represented by SEQ ID NOS: 68579, 68580, 68581, 68582, 68583, 68584, 68585, 68586, 68587, 68588, 68589 were selected from the ORFs having the nucleotide sequence represented by SEQ ID NO: 5633.

The probes having the nucleotide sequence represented by SEQ ID NOS: 68590, 68591, 68592, 68593, 68594, 68595, 68596, 68597, 68598, 68599, 68600 were selected from the ORFs having the nucleotide sequence represented by SEQ ID NO: 5634.

The probes having the nucleotide sequence represented by SEQ ID NOS: 68601, 68602, 68603, 68604, 68605, 68606, 68607, 68608, 68609, 68610, 68611 were selected from the ORFs having the nucleotide sequence represented by SEQ ID NO: 5635.

The probes having the nucleotide sequence represented by SEQ ID NOS: 68612, 68613, 68614, 68615, 68616, 68617, 68618, 68619, 68620, 68621, 68622 were selected from the ORFs having the nucleotide sequence represented by SEQ ID NO: 5636.

The probes having the nucleotide sequence represented by SEQ ID NOS: 68623, 68624, 68625, 68626, 68627, 68628, 68629, 68630, 68631, 68632, 68633 were selected from the ORFs having the nucleotide sequence represented by SEQ ID NO: 5637.

The probes having the nucleotide sequence represented by SEQ ID NOS: 68634, 68635, 68636, 68637, 68638, 68639, 68640, 68641, 68642, 68643, 68644 were selected from the ORFs having the nucleotide sequence represented by SEQ ID NO: 5638.

The probes having the nucleotide sequence represented by SEQ ID NOS: 68645, 68646, 68647, 68648, 68649, 68650, 68651, 68652, 68653, 68654, 68655 were selected from the ORFs having the nucleotide sequence represented by SEQ ID NO: 5639.

The probes having the nucleotide sequence represented by SEQ ID NOS: 68656, 68657, 68658, 68659, 68660, 68661, 68662, 68663, 68664, 68665, 68666 were selected from the ORFs having the nucleotide sequence represented by SEQ ID NO: 5640.

The probes having the nucleotide sequence represented by SEQ ID NOS: 68667, 68668, 68669, 68670, 68671, 68672, 68673, 68674, 68675, 68676, 68677 were selected from the ORFs having the nucleotide sequence represented by SEQ ID NO: 5641.

The probes having the nucleotide sequence represented by SEQ ID NOS: 68678, 68679, 68680, 68681, 68682, 68683, 68684, 68685, 68686, 68687, 68688 were selected from the ORFs having the nucleotide sequence represented by SEQ ID NO: 5642.

The probes having the nucleotide sequence represented by SEQ ID NOS: 68689, 68690, 68691, 68692, 68693, 68694, 68695, 68696, 68697, 68698, 68699 were selected from the ORFs having the nucleotide sequence represented by SEQ ID NO: 5643.

The probes having the nucleotide sequence represented by SEQ ID NOS: 68700, 68701, 68702, 68703, 68704, 68705, 68706, 68707, 68708, 68709, 68710 were selected from the ORFs having the nucleotide sequence represented by SEQ ID NO: 5644.

The probes having the nucleotide sequence represented by SEQ ID NOS: 68711, 68712, 68713, 68714, 68715, 68716, 68717, 68718, 68719, 68720, 68721 were selected from the ORFs having the nucleotide sequence represented by SEQ ID NO: 5645.

The probes having the nucleotide sequence represented by SEQ ID NOS: 68722, 68723, 68724, 68725, 68726, 68727, 68728, 68729, 68730, 68731, 68732 were selected from the ORFs having the nucleotide sequence represented by SEQ ID NO: 5646.

The probes having the nucleotide sequence represented by SEQ ID NOS: 68733, 68734, 68735, 68736, 68737, 68738, 68739, 68740, 68741, 68742, 68743 were selected from the ORFs having the nucleotide sequence represented by SEQ ID NO: 5647.

The probes having the nucleotide sequence represented by SEQ ID NOS: 68744, 68745, 68746, 68747, 68748, 68749, 68750, 68751, 68752, 68753, 68754 were selected from the ORFs having the nucleotide sequence represented by SEQ ID NO: 5648.

The probes having the nucleotide sequence represented by SEQ ID NOS: 68755, 68756, 68757, 68758, 68759, 68760, 68761, 68762, 68763, 68764, 68765 were selected from the ORFs having the nucleotide sequence represented by SEQ ID NO: 5649.

The probes having the nucleotide sequence represented by SEQ ID NOS: 68766, 68767, 68768, 68769, 68770, 68771, 68772, 68773, 68774, 68775, 68776 were selected from the ORFs having the nucleotide sequence represented by SEQ ID NO: 5650.

The probes having the nucleotide sequence represented by SEQ ID NOS: 68777, 68778, 68779, 68780, 68781, 68782, 68783, 68784, 68785, 68786, 68787 were selected from the ORFs having the nucleotide sequence represented by SEQ ID NO: 5651.

The probes having the nucleotide sequence represented by SEQ ID NOS: 68788, 68789, 68790, 68791, 68792, 68793, 68794, 68795, 68796, 68797, 68798 were selected from the ORFs having the nucleotide sequence represented by SEQ ID NO: 5652.

The probes having the nucleotide sequence represented by SEQ ID NOS: 68799, 68800, 68801, 68802, 68803, 68804, 68805, 68806, 68807, 68808, 68809 were selected from the ORFs having the nucleotide sequence represented by SEQ ID NO: 5653.

The probes having the nucleotide sequence represented by SEQ ID NOS: 68810, 68811, 68812, 68813, 68814, 68815, 68816, 68817, 68818, 68819, 68820 were selected from the ORFs having the nucleotide sequence represented by SEQ ID NO: 5654.

The probes having the nucleotide sequence represented by SEQ ID NOS: 68821, 68822, 68823, 68824, 68825, 68826, 68827, 68828, 68829, 68830, 68831 were selected from the ORFs having the nucleotide sequence represented by SEQ ID NO: 5655.

The probes having the nucleotide sequence represented by SEQ ID NOS: 68832, 68833, 68834, 68835, 68836, 68837, 68838, 68839, 68840, 68841, 68842 were selected from the ORFs having the nucleotide sequence represented by SEQ ID NO: 5656.

The probes having the nucleotide sequence represented by SEQ ID NOS: 68843, 68844, 68845, 68846, 68847, 68848, 68849, 68850, 68851, 68852, 68853 were selected from the ORFs having the nucleotide sequence represented by SEQ ID NO: 5657.

The probes having the nucleotide sequence represented by SEQ ID NOS: 68854, 68855, 68856, 68857, 68858, 68859, 68860, 68861, 68862, 68863, 68864 were selected from the ORFs having the nucleotide sequence represented by SEQ ID NO: 5658.

The probes having the nucleotide sequence represented by SEQ ID NOS: 68865, 68866, 68867, 68868, 68869, 68870, 68871, 68872, 68873, 68874, 68875 were selected from the ORFs having the nucleotide sequence represented by SEQ ID NO: 5659.

The probes having the nucleotide sequence represented by SEQ ID NOS: 68876, 68877, 68878, 68879, 68880, 68881, 68882, 68883, 68884, 68885, 68886 were selected from the ORFs having the nucleotide sequence represented by SEQ ID NO: 5660.

The probes having the nucleotide sequence represented by SEQ ID NOS: 68887, 68888, 68889, 68890, 68891, 68892, 68893, 68894, 68895, 68896, 68897 were selected from the ORFs having the nucleotide sequence represented by SEQ ID NO: 5661.

The probes having the nucleotide sequence represented by SEQ ID NOS: 68898, 68899, 68900, 68901, 68902, 68903, 68904, 68905, 68906, 68907, 68908 were selected from the ORFs having the nucleotide sequence represented by SEQ ID NO: 5662.

The probes having the nucleotide sequence represented by SEQ ID NOS: 68909, 68910, 68911, 68912, 68913, 68914, 68915, 68916, 68917, 68918, 68919 were selected from the ORFs having the nucleotide sequence represented by SEQ ID NO: 5663.

The probes having the nucleotide sequence represented by SEQ ID NOS: 68920, 68921, 68922, 68923, 68924, 68925, 68926, 68927, 68928, 68929, 68930 were selected from the ORFs having the nucleotide sequence represented by SEQ ID NO: 5664.

The probes having the nucleotide sequence represented by SEQ ID NOS: 68931, 68932, 68933, 68934, 68935, 68936, 68937, 68938, 68939, 68940, 68941 were selected from the ORFs having the nucleotide sequence represented by SEQ ID NO: 5665.

The probes having the nucleotide sequence represented by SEQ ID NOS: 68942, 68943, 68944, 68945, 68946, 68947, 68948, 68949, 68950, 68951, 68952 were selected from the ORFs having the nucleotide sequence represented by SEQ ID NO: 5666.

The probes having the nucleotide sequence represented by SEQ ID NOS: 68953, 68954, 68955, 68956, 68957, 68958, 68959, 68960, 68961, 68962, 68963 were selected from the ORFs having the nucleotide sequence represented by SEQ ID NO: 5667.

The probes having the nucleotide sequence represented by SEQ ID NOS: 68964, 68965, 68966, 68967, 68968, 68969, 68970, 68971, 68972, 68973, 68974 were selected from the ORFs having the nucleotide sequence represented by SEQ ID NO: 5668.

The probes having the nucleotide sequence represented by SEQ ID NOS: 68975, 68976, 68977, 68978, 68979, 68980, 68981, 68982, 68983, 68984, 68985 were selected from the ORFs having the nucleotide sequence represented by SEQ ID NO: 5669.

The probes having the nucleotide sequence represented by SEQ ID NOS: 68986, 68987, 68988, 68989, 68990, 68991, 68992, 68993, 68994, 68995, 68996 were selected from the ORFs having the nucleotide sequence represented by SEQ ID NO: 5670.

The probes having the nucleotide sequence represented by SEQ ID NOS: 68997, 68998, 68999, 69000, 69001, 69002, 69003, 69004, 69005, 69006, 69007 were selected from the ORFs having the nucleotide sequence represented by SEQ ID NO: 5671.

The probes having the nucleotide sequence represented by SEQ ID NOS: 69008, 69009, 69010, 69011, 69012, 69013, 69014, 69015, 69016, 69017, 69018 were selected from the ORFs having the nucleotide sequence represented by SEQ ID NO: 5672.

The probes having the nucleotide sequence represented by SEQ ID NOS: 69019, 69020, 69021, 69022, 69023, 69024, 69025, 69026, 69027, 69028, 69029 were selected from the ORFs having the nucleotide sequence represented by SEQ ID NO: 5673.

The probes having the nucleotide sequence represented by SEQ ID NOS: 69030, 69031, 69032, 69033, 69034, 69035, 69036, 69037, 69038, 69039, 69040 were selected from the ORFs having the nucleotide sequence represented by SEQ ID NO: 5674.

The probes having the nucleotide sequence represented by SEQ ID NOS: 69041, 69042, 69043, 69044, 69045, 69046, 69047, 69048, 69049, 69050, 69051 were selected from the ORFs having the nucleotide sequence represented by SEQ ID NO: 5675.

The probes having the nucleotide sequence represented by SEQ ID NOS: 69052, 69053, 69054, 69055, 69056, 69057, 69058, 69059, 69060, 69061, 69062 were selected from the ORFs having the nucleotide sequence represented by SEQ ID NO: 5676.

The probes having the nucleotide sequence represented by SEQ ID NOS: 69063, 69064, 69065, 69066, 69067, 69068, 69069, 69070, 69071, 69072, 69073 were selected from the ORFs having the nucleotide sequence represented by SEQ ID NO: 5677.

The probes having the nucleotide sequence represented by SEQ ID NOS: 69074, 69075, 69076, 69077, 69078, 69079, 69080, 69081, 69082, 69083, 69084 were selected from the ORFs having the nucleotide sequence represented by SEQ ID NO: 5678.

The probes having the nucleotide sequence represented by SEQ ID NOS: 69085, 69086, 69087, 69088, 69089, 69090, 69091, 69092, 69093, 69094, 69095 were selected from the ORFs having the nucleotide sequence represented by SEQ ID NO: 5679.

The probes having the nucleotide sequence represented by SEQ ID NOS: 69096, 69097, 69098, 69099, 69100, 69101, 69102, 69103, 69104, 69105, 69106 were selected from the ORFs having the nucleotide sequence represented by SEQ ID NO: 5680.

The probes having the nucleotide sequence represented by SEQ ID NOS: 69107, 69108, 69109, 69110, 69111, 69112, 69113, 69114, 69115, 69116, 69117 were selected from the ORFs having the nucleotide sequence represented by SEQ ID NO: 5681.

The probes having the nucleotide sequence represented by SEQ ID NOS: 69118, 69119, 69120, 69121, 69122, 69123, 69124, 69125, 69126, 69127, 69128 were selected from the ORFs having the nucleotide sequence represented by SEQ ID NO: 5682.

The probes having the nucleotide sequence represented by SEQ ID NOS: 69129, 69130, 69131, 69132, 69133, 69134, 69135, 69136, 69137, 69138, 69139 were selected from the ORFs having the nucleotide sequence represented by SEQ ID NO: 5683.

The probes having the nucleotide sequence represented by SEQ ID NOS: 69140, 69141, 69142, 69143, 69144, 69145, 69146, 69147, 69148, 69149, 69150 were selected from the ORFs having the nucleotide sequence represented by SEQ ID NO: 5684.

The probes having the nucleotide sequence represented by SEQ ID NOS: 69151, 69152, 69153, 69154, 69155, 69156,

69157, 69158, 69159, 69160, 69161 were selected from the ORFs having the nucleotide sequence represented by SEQ ID NO: 5685.

The probes having the nucleotide sequence represented by SEQ ID NOS: 69162, 69163, 69164, 69165, 69166, 69167, 69168, 69169, 69170, 69171, 69172 were selected from the ORFs having the nucleotide sequence represented by SEQ ID NO: 5686.

The probes having the nucleotide sequence represented by SEQ ID NOS: 69173, 69174, 69175, 69176, 69177, 69178, 69179, 69180, 69181, 69182, 69183 were selected from the ORFs having the nucleotide sequence represented by SEQ ID NO: 5687.

The probes having the nucleotide sequence represented by SEQ ID NOS: 69184, 69185, 69186, 69187, 69188, 69189, 69190, 69191, 69192, 69193, 69194 were selected from the ORFs having the nucleotide sequence represented by SEQ ID NO: 5688.

The probes having the nucleotide sequence represented by SEQ ID NOS: 69195, 69196, 69197, 69198, 69199, 69200, 69201, 69202, 69203, 69204, 69205 were selected from the ORFs having the nucleotide sequence represented by SEQ ID NO: 5689.

The probes having the nucleotide sequence represented by SEQ ID NOS: 69206, 69207, 69208, 69209, 69210, 69211, 69212, 69213, 69214, 69215, 69216 were selected from the ORFs having the nucleotide sequence represented by SEQ ID NO: 5690.

The probes having the nucleotide sequence represented by SEQ ID NOS: 69217, 6921.8, 69219, 69220, 69221, 69222, 69223, 69224, 69225, 69226, 69227 were selected from the ORFs having the nucleotide sequence represented by SEQ ID NO: 5691.

The probes having the nucleotide sequence represented by SEQ ID NOS: 69228, 69229, 69230, 69231, 69232, 69233, 69234, 69235, 69236, 69237, 69238 were selected from the ORFs having the nucleotide sequence represented by SEQ ID NO: 5692.

The probes having the nucleotide sequence represented by SEQ ID NOS: 69239, 69240, 69241, 69242, 69243, 69244, 69245, 69246, 69247, 69248, 69249 were selected from the ORFs having the nucleotide sequence represented by SEQ ID NO: 5693.

The probes having the nucleotide sequence represented by SEQ ID NOS: 69250, 69251, 69252, 69253, 69254, 69255, 69256, 69257, 69258, 69259, 69260 were selected from the ORFs having the nucleotide sequence represented by SEQ ID NO: 5694.

The probes having the nucleotide sequence represented by SEQ ID NOS: 69261, 69262, 69263, 69264, 69265, 69266, 69267, 69268, 69269, 69270, 69271 were selected from the ORFs having the nucleotide sequence represented by SEQ ID NO: 5695.

The probes having the nucleotide sequence represented by SEQ ID NOS: 69272, 69273, 69274, 69275, 69276, 69277, 69278, 69279, 69280, 69281, 69282 were selected from the ORFs having the nucleotide sequence represented by SEQ ID NO: 5696.

The probes having the nucleotide sequence represented by SEQ ID NOS: 69283, 69284, 69285, 69286, 69287, 69288, 69289, 69290, 69291, 69292, 69293 were selected from the ORFs having the nucleotide sequence represented by SEQ ID NO: 5697.

The probes having the nucleotide sequence represented by SEQ ID NOS: 69294, 69295, 69296, 69297, 69298, 69299, 69300, 69301, 69302, 69303, 69304 were selected from the ORFs having the nucleotide sequence represented by SEQ ID NO: 5698.

The probes having the nucleotide sequence represented by SEQ ID NOS: 69305, 69306, 69307, 69308, 69309, 69310, 69311, 69312, 69313, 69314, 69315 were selected from the ORFs having the nucleotide sequence represented by SEQ ID NO: 5699.

The probes having the nucleotide sequence represented by SEQ ID NOS: 69316, 69317, 69318, 69319, 69320, 69321, 69322, 69323, 69324, 69325, 69326 were selected from the ORFs having the nucleotide sequence represented by SEQ ID NO: 5700.

The probes having the nucleotide sequence represented by SEQ ID NOS: 69327, 69328, 69329, 69330, 69331, 69332, 69333, 69334, 69335, 69336, 69337, 69338, 69339, 69340, 69341, 69342, 69343, 69344, 69345, 69346, 69347, 69348 were selected from the ORFs having the nucleotide sequence represented by SEQ ID NO: 5701.

The probes having the nucleotide sequence represented by SEQ ID NOS: 69349, 69350, 69351, 69352, 69353, 69354, 69355, 69356, 69357, 69358, 69359 were selected from the ORFs having the nucleotide sequence represented by SEQ ID NO: 5702.

The probes having the nucleotide sequence represented by SEQ ID NOS: 69360, 69361, 69362, 69363, 69364, 69365, 69366, 69367, 69368, 69369, 69370 were selected from the ORFs having the nucleotide sequence represented by SEQ ID NO: 5703.

The probes having the nucleotide sequence represented by SEQ ID NOS: 69371, 69372, 69373, 69374, 69375, 69376, 69377, 69378, 69379, 69380, 69381 were selected from the ORFs having the nucleotide sequence represented by SEQ ID NO: 5704.

The probes having the nucleotide sequence represented by SEQ ID NOS: 69382, 69383, 69384, 69385, 69386, 69387, 69388, 69389, 69390, 69391, 69392 were selected from the ORFs having the nucleotide sequence represented by SEQ ID NO: 5705.

The probes having the nucleotide sequence represented by SEQ ID NOS: 69393, 69394, 69395, 69396, 69397, 69398, 69399, 69400, 69401, 69402, 69403 were selected from the ORFs having the nucleotide sequence represented by SEQ ID NO: 5706.

The probes having the nucleotide sequence represented by SEQ ID NOS: 69404, 69405, 69406, 69407, 69408, 69409, 69410, 69411, 69412, 69413, 69414 were selected from the ORFs having the nucleotide sequence represented by SEQ ID NO: 5707.

The probes having the nucleotide sequence represented by SEQ ID NOS: 69415, 69416, 69417, 69418, 69419, 69420, 69421, 69422, 69423, 69424, 69425 were selected from the ORFs having the nucleotide sequence represented by SEQ ID NO: 5708.

The probes having the nucleotide sequence represented by SEQ ID NOS: 69426, 69427, 69428, 69429, 69430, 69431, 69432, 69433, 69434, 69435, 69436 were selected from the ORFs having the nucleotide sequence represented by SEQ ID NO: 5709.

The probes having the nucleotide sequence represented by SEQ ID NOS: 69437, 69438, 69439, 69440, 69441, 69442, 69443, 69444, 69445, 69446, 69447 were selected from-the ORFs having the nucleotide sequence represented by SEQ ID NO: 5710.

The probes having the nucleotide sequence represented by SEQ ID NOS: 69448, 69449, 69450, 69451, 69452, 69453, 69454, 69455, 69456, 69457, 69458 were selected from the ORFs having the nucleotide sequence represented by SEQ ID NO: 5711.

The probes having the nucleotide sequence represented by SEQ ID NOS: 69459, 69460, 69461, 69462, 69463, 69464, 69465, 69466, 69467, 69468, 69469 were selected from the ORFs having the nucleotide sequence represented by SEQ ID NO: 5712.

The probes having the nucleotide sequence represented by SEQ ID NOS: 69470, 69471, 69472, 69473, 69474, 69475, 69476, 69477, 69478, 69479, 69480 were selected from the ORFs having the nucleotide sequence represented by SEQ ID NO: 5713.

The probes having the nucleotide sequence represented by SEQ ID NOS: 69481, 69482, 69483, 69484, 69485, 69486, 69487, 69488, 69489, 69490, 69491 were selected from the ORFs having the nucleotide sequence represented by SEQ ID NO: 5714.

The probes having the nucleotide sequence represented by SEQ ID NOS: 69492, 69493, 69494, 69495, 69496, 69497, 69498, 69499, 69500, 69501, 69502 were selected from the ORFs having the nucleotide sequence represented by SEQ ID NO: 5715.

The probes having the nucleotide sequence represented by SEQ ID NOS: 69503, 69504, 69505, 69506, 69507, 69508, 69509, 69510, 69511, 69512, 69513 were selected from the ORFs having the nucleotide sequence represented by SEQ ID NO: 5716.

The probes having the nucleotide sequence represented by SEQ ID NOS: 69514, 69515, 69516, 69517, 69518, 69519, 69520, 69521, 69522, 69523, 69524 were selected from the ORFs having the nucleotide sequence represented by SEQ ID NO: 5717.

The probes having the nucleotide sequence represented by SEQ ID NOS: 69525, 69526, 69527, 69528, 69529, 69530, 69531, 69532, 69533, 69534, 69535 were selected from the ORFs having the nucleotide sequence represented by SEQ ID NO: 5718.

The probes having the nucleotide sequence represented by SEQ ID NOS: 69536, 69537, 69538, 69539, 69540, 69541, 69542, 69543, 69544, 69545, 69546 were selected from the ORFs having the nucleotide sequence represented by SEQ ID NO: 5719.

The probes having the nucleotide sequence represented by SEQ ID NOS: 69547, 69548, 69549, 69550, 69551, 69552, 69553, 69554, 69555, 69556, 69557 were selected from the ORFs having the nucleotide sequence represented by SEQ ID NO: 5720.

The probes having the nucleotide sequence represented by SEQ ID NOS: 69558, 69559, 69560, 69561, 69562, 69563, 69564, 69565, 69566, 69567, 69568 were selected from the ORFs having the nucleotide sequence represented by SEQ ID NO: 5721.

The probes having the nucleotide sequence represented by SEQ ID NOS: 69569, 69570, 69571, 69572, 69573, 69574, 69575, 69576, 69577, 69578, 69579 were selected from the ORFs having the nucleotide sequence represented by SEQ ID NO: 5722.

The probes having the nucleotide sequence represented by SEQ ID NOS: 69580, 69581, 69582, 69583, 69584, 69585, 69586, 69587, 69588, 69589, 69590 were selected from the ORFs having the nucleotide sequence represented by SEQ ID NO: 5723.

The probes having the nucleotide sequence represented by SEQ ID NOS: 69591, 69592, 69593, 69594, 69595, 69596, 69597, 69598, 69599, 69600, 69601 were selected from the ORFs having the nucleotide sequence represented by SEQ ID NO: 5724.

The probes having the nucleotide sequence represented by SEQ ID NOS: 69602, 69603, 69604, 69605, 69606, 69607, 69608, 69609, 69610, 69611, 69612 were selected from the ORFs having the nucleotide sequence represented by SEQ ID NO: 5725.

The probes having the nucleotide sequence represented by SEQ ID NOS: 69613, 69614, 69615, 69616, 69617, 69618, 69619, 69620, 69621, 69622, 69623 were selected from the ORFs having the nucleotide sequence represented by SEQ ID NO: 5726.

The probes having the nucleotide sequence represented by SEQ ID NOS: 69624, 69625, 69626, 69627, 69628, 69629, 69630, 69631, 69632, 69633, 69634 were selected from the ORFs having the nucleotide sequence represented by SEQ ID NO: 5727.

The probes having the nucleotide sequence represented by SEQ ID NOS: 69635, 69636, 69637, 69638, 69639, 69640, 69641, 69642, 69643, 69644, 69645 were selected from the ORFs having the nucleotide sequence represented by SEQ ID NO: 5728.

The probes having the nucleotide sequence represented by SEQ ID NOS: 69646, 69647, 69648, 69649, 69650, 69651, 69652, 69653, 69654, 69655, 69656 were selected from the ORFs having the nucleotide sequence represented by SEQ ID NO: 5729.

The probes having the nucleotide sequence represented by SEQ ID NOS: 69657, 69658, 69659, 69660, 69661, 69662, 69663, 69664, 69665, 69666, 69667 were selected from the ORFs having the nucleotide sequence represented by SEQ ID NO: 5730.

The probes having the nucleotide sequence represented by SEQ ID NOS: 69668, 69669, 69670, 69671, 69672, 69673, 69674, 69675, 69676, 69677, 69678 were selected from the ORFs having the nucleotide sequence represented by SEQ ID NO: 5731.

The probes having the nucleotide sequence represented by SEQ ID NOS: 69679, 69680, 69681, 69682, 69683, 69684, 69685, 69686, 69687, 69688, 69689 were selected from the ORFs having the nucleotide sequence represented by SEQ ID NO: 5732.

The probes having the nucleotide sequence represented by SEQ ID NOS: 69690, 69691, 69692, 69693, 69694, 69695, 69696, 69697, 69698, 69699, 69700 were selected from the ORFs having the nucleotide sequence represented by SEQ ID NO: 5733.

The probes having the nucleotide sequence represented by SEQ ID NOS: 69701, 69702, 69703, 69704, 69705, 69706, 69707, 69708, 69709, 69710, 69711 were selected from the ORFs having the nucleotide sequence represented by SEQ ID NO: 5734.

The probes having the nucleotide sequence represented by SEQ ID NOS: 69712, 69713, 69714, 69715, 69716, 69717, 69718, 69719, 69720, 69721, 69722 were selected from the ORFs having the nucleotide sequence represented by SEQ ID NO: 5735.

The probes having the nucleotide sequence represented by SEQ ID NOS: 69723, 69724, 69725, 69726, 69727, 69728, 69729, 69730, 69731, 69732, 69733 were selected from the ORFs having the nucleotide sequence represented by SEQ ID NO: 5736.

The probes having the nucleotide sequence represented by SEQ ID NOS: 69734, 69735, 69736, 69737, 69738, 69739, 69740, 69741, 69742, 69743, 69744 were selected from the ORFs having the nucleotide sequence represented by SEQ ID NO: 5737.

The probes having the nucleotide sequence represented by SEQ ID NOS: 69745, 69746, 69747, 69748, 69749, 69750, 6,9751, 69752, 69753, 69754, 69755 were selected from the ORFs having the nucleotide sequence represented by SEQ ID NO: 5738.

The probes having the nucleotide sequence represented by SEQ ID NOS: 69756, 69757, 69758, 69759, 69760, 69761, 69762, 69763, 69764, 69765, 69766 were selected from the ORFs having the nucleotide sequence represented by SEQ ID NO: 5739.

The probes having the nucleotide sequence represented by SEQ ID NOS: 69767, 69768, 69769, 69770, 69771, 69772, 69773, 69774, 69775, 69776, 69777 were selected from the ORFs having the nucleotide sequence represented by SEQ ID NO: 5740.

The probes having the nucleotide sequence represented by SEQ ID NOS: 69778, 69779, 69780, 69781, 69782, 69783, 69784, 69785, 69786, 69787, 69788 were selected from the ORFs having the nucleotide sequence represented by SEQ ID NO: 5741.

The probes having the nucleotide sequence represented by SEQ ID NOS: 69789, 69790, 69791, 69792, 69793, 69794, 69795, 69796, 69797, 69798, 69799 were selected from the ORFs having the nucleotide sequence represented by SEQ ID NO: 5742.

The probes having the nucleotide sequence represented by SEQ ID NOS: 69800, 69801, 69802, 69803, 69804, 69805, 69806, 69807, 69808, 69809, 69810 were selected from the ORFs having the nucleotide sequence represented by SEQ ID NO: 5743.

The probes having the nucleotide sequence represented by SEQ ID NOS: 69811, 69812, 69813, 69814, 69815, 69816, 69817, 69818, 69819, 69820, 69821 were selected from the ORFs having the nucleotide sequence represented by SEQ ID NO: 5744.

The probes having the nucleotide sequence represented by SEQ ID NOS: 69822, 69823, 69824, 69825, 69826, 69827, 69828, 69829, 69830, 69831, 69832 were selected from the ORFs having the nucleotide sequence represented by SEQ ID NO: 5745.

The probes having the nucleotide sequence represented by SEQ ID NOS: 69833, 69834, 69835, 69836, 69837, 69838, 69839, 69840, 69841, 69842, 69843 were selected from the ORFs having the nucleotide sequence represented by SEQ ID NO: 5746.

The probes having the nucleotide sequence represented by SEQ ID NOS: 69844, 69845, 69846, 69847, 69848, 69849, 69850, 69851, 69852, 69853, 69854 were selected from the ORFs having the nucleotide sequence represented by SEQ ID NO: 5747.

The probes having the nucleotide sequence represented by SEQ ID NOS: 69855, 69856, 69857, 69858, 69859, 69860, 69861, 69862, 69863, 69864, 69865 were selected from the ORFs having the nucleotide sequence represented by SEQ ID NO: 5748.

The probes having the nucleotide sequence represented by SEQ ID NOS: 69866, 69867, 69868, 69869, 69870, 69871, 69872, 69873, 69874, 69875, 69876 were selected from the ORFs having the nucleotide sequence represented by SEQ ID NO: 5749.

The probes having the nucleotide sequence represented by SEQ ID NOS: 69877, 69878, 69879, 69880, 69881, 69882, 69883, 69884, 69885, 69886, 69887 were selected from the ORFs having the nucleotide sequence represented by SEQ ID NO: 5750.

The probes having the nucleotide sequence represented by SEQ ID NOS: 69888, 69889, 69890, 69891, 69892, 69893, 69894, 69895, 69896, 69897, 69898 were selected from the ORFs having the nucleotide sequence represented by SEQ ID NO: 5751.

The probes having the nucleotide sequence represented by SEQ ID NOS: 69899, 69900, 69901, 69902, 69903, 69904, 69905, 69906, 69907, 69908, 69909 were selected from the ORFs having the nucleotide sequence represented by SEQ ID NO: 5752.

The probes having the nucleotide sequence represented by SEQ ID NOS: 69910, 69911, 69912, 69913, 69914, 69915, 69916, 69917, 69918, 69919, 69920 were selected from the ORFs having the nucleotide sequence represented by SEQ ID NO: 5753.

The probes having the nucleotide sequence represented by SEQ ID NOS: 69921, 69922, 69923, 69924, 69925, 69926, 69927, 69928, 69929, 69930, 69931 were selected from the ORFs having the nucleotide sequence represented by SEQ ID NO: 5754.

The probes having the nucleotide sequence represented by SEQ ID NOS: 69932, 69933, 69934, 69935, 69936, 69937, 69938, 69939, 69940, 69941, 69942 were selected from the ORFs having the nucleotide sequence represented by SEQ ID NO: 5755.

The probes having the nucleotide sequence represented by SEQ ID NOS: 69943, 69944, 69945, 69946, 69947, 69948, 69949, 69950, 69951, 69952, 69953 were selected from the ORFs having the nucleotide sequence represented by SEQ ID NO: 5756.

The probes having the nucleotide sequence represented by SEQ ID NOS: 69954, 69955, 69956, 69957, 69958, 69959, 69960, 69961, 69962, 69963, 69964 were selected from the ORFs having the nucleotide sequence represented by SEQ ID NO: 5757.

The probes having the nucleotide sequence represented by SEQ ID NOS: 69965, 69966, 69967, 69968, 69969, 69970, 69971, 69972, 69973, 69974, 69975 were selected from the ORFs having the nucleotide sequence represented by SEQ ID NO: 5758.

The probes having the nucleotide sequence represented by SEQ ID NOS: 69976, 69977, 69978, 69979, 69980, 69981, 69982, 69983, 69984, 69985, 69986, 69987, 69988, 69989, 69990, 69991, 69992, 69993, 69994, 69995, 69996, 69997 were selected from the ORFs having the nucleotide sequence represented by SEQ ID NO: 5759.

The probes having the nucleotide sequence represented by SEQ ID NOS: 69998, 69999, 70000, 70001, 70002, 70003, 70004, 70005, 70006, 70007, 70008 were selected from the ORFs having the nucleotide sequence represented by SEQ ID NO: 5760.

The probes having the nucleotide sequence represented by SEQ ID NOS: 70009, 70010, 70011, 70012, 70013, 70014, 70015, 70016, 70017, 70018, 70019 were selected from the ORFs having the nucleotide sequence represented by SEQ ID NO: 5761.

The probes having the nucleotide sequence represented by SEQ ID NOS: 70020, 70021, 70022, 70023, 70024, 70025, 70026, 70027, 70028, 70029, 70030 were selected from the ORFs having the nucleotide sequence represented by SEQ ID NO: 5762.

The probes having the nucleotide sequence represented by SEQ ID NOS: 70031, 70032, 70033, 70034, 70035, 70036, 70037, 70038, 70039, 70040, 70041 were selected from the ORFs having the nucleotide sequence represented by SEQ ID NO: 5763.

The probes having the nucleotide sequence represented by SEQ ID NOS: 70042, 70043, 70044, 70045, 70046, 70047, 70048, 70049, 70050, 70051, 70052 were selected from the ORFs having the nucleotide sequence represented by SEQ ID NO: 5764.

The probes having the nucleotide sequence represented by SEQ ID NOS: 70053, 70054, 70055, 70056, 70057, 70058, 70059, 70060, 70061, 70062, 70063 were selected from the ORFs having the nucleotide sequence represented by SEQ ID NO: 5765.

The probes having the nucleotide sequence represented by SEQ ID NOS: 70064, 70065, 70066, 70067, 70068, 70069, 70070, 70071, 70072, 70073, 70074 were selected from the ORFs having the nucleotide sequence represented by SEQ ID NO: 5766.

The probes having the nucleotide sequence represented by SEQ ID NOS: 70075, 70076, 70077, 70078, 70079, 70080, 70081, 70082, 70083, 70084, 70085 were selected from the ORFs having the nucleotide sequence represented by SEQ ID NO: 5767.

The probes having the nucleotide sequence represented by SEQ ID NOS: 70086, 70087, 70088, 70089, 70090, 70091, 70092, 70093, 70094, 70095, 70096 were selected from the ORFs having the nucleotide sequence represented by SEQ ID NO: 5768.

The probes having the nucleotide sequence represented by SEQ ID NOS: 70097, 70098, 70099, 70100, 70101, 70102, 70103, 70104, 70105, 70106, 70107 were selected from the ORFs having the nucleotide sequence represented by SEQ ID NO: 5769.

The probes having the nucleotide sequence represented by SEQ ID NOS: 70108, 70109, 70110, 70111, 70112, 70113, 70114, 70115, 70116, 70117, 70118, 70119, 70120, 70121, 70122, 70123, 70124, 70125, 70126, 70127, 70128, 70129 were selected from the ORFs having the nucleotide sequence represented by SEQ ID NO: 5770.

The probes having the nucleotide sequence represented by SEQ ID NOS: 70130, 70131, 70132, 70133, 70134, 70135, 70136, 70137, 70138, 70139, 70140 were selected from the ORFs having the nucleotide sequence represented by SEQ ID NO: 5771.

The probes having the nucleotide sequence represented by SEQ ID NOS: 70141, 70142, 70143, 70144, 70145, 70146, 70147, 70148 were selected from the ORFs having the nucleotide sequence represented by SEQ ID NO: 5772.

The probes having the nucleotide sequence represented by SEQ ID NOS: 70149, 70150, 70151, 70152, 70153, 70154, 70155, 70156, 70157, 70158, 70159 were selected from the ORFs having the nucleotide sequence represented by SEQ ID NO: 5773.

The probes having the nucleotide sequence represented by SEQ ID NOS: 70160, 70161, 70162, 70163, 70164, 70165, 70166, 70167, 70168, 70169, 70170 were selected from the ORFs having the nucleotide sequence represented by SEQ ID NO: 5774.

The probes having the nucleotide sequence represented by SEQ ID NOS: 70171, 70172, 70173, 70174, 70175, 70176, 70177, 70178, 70179, 70180, 70181 were selected from the ORFs having the nucleotide sequence represented by SEQ ID NO: 5775.

The probes having the nucleotide sequence represented by SEQ ID NOS: 70182, 70183, 70184, 70185, 70186, 70187, 70188, 70189, 70190, 70191, 70192 were selected from the ORFs having the nucleotide sequence represented by SEQ ID NO: 5776.

The probes having the nucleotide sequence represented by SEQ ID NOS: 70193, 70194, 70195, 70196, 70197, 70198, 70199, 70200, 70201, 70202, 70203 were selected from the ORFs having the nucleotide sequence represented by SEQ ID NO: 5777.

The probes having the nucleotide sequence represented by SEQ ID NOS: 70204, 70205, 70206, 70207, 70208, 70209, 70210, 70211, 70212, 70213, 70214 were selected from the ORFs having the nucleotide sequence represented by SEQ ID NO: 5778.

The probes having the nucleotide sequence represented by SEQ ID NOS: 70215, 70216, 70217, 70218, 70219, 70220, 70221, 70222, 70223, 70224, 70225 were selected from the ORFs having the nucleotide sequence represented by SEQ ID NO: 5779.

The probes having the nucleotide sequence represented by SEQ ID NOS: 70226, 70227, 70228, 70229, 70230, 70231, 70232, 70233, 70234, 70235, 70236 were selected from the ORFs having the nucleotide sequence represented by SEQ ID NO: 5780.

The probes having the nucleotide sequence represented by SEQ ID NOS: 70237, 70238, 70239, 70240, 70241, 70242, 70243, 70244, 70245, 70246, 70247 were selected from the ORFs having the nucleotide sequence represented by SEQ ID NO: 5781.

The probes having the nucleotide sequence represented by SEQ ID NOS: 70248, 70249, 70250, 70251, 70252, 70253, 70254, 70255, 70256, 70257, 70258 were selected from the ORFs having the nucleotide sequence represented by SEQ ID NO: 5782.

The probes having the nucleotide sequence represented by SEQ ID NOS: 70259, 70260, 70261, 70262, 70263, 70264, 70265, 70266, 70267, 70268, 70269 were selected from the ORFs having the nucleotide sequence represented by SEQ ID NO: 5783.

The probes having the nucleotide sequence represented by SEQ ID NOS: 70270, 70271, 70272, 70273, 70274, 70275, 70276, 70277, 70278, 70279, 70280 were selected from the ORFs having the nucleotide sequence represented by SEQ ID NO: 5784.

The probes having the nucleotide sequence represented by SEQ ID NOS: 70281, 70282, 70283, 70284, 70285, 70286, 70287, 70288, 70289, 70290, 70291 were selected from the ORFs having the nucleotide sequence represented by SEQ ID NO: 5785.

The probes having the nucleotide sequence represented by SEQ ID NOS: 70292, 70293, 70294, 70295, 70296, 70297, 70298, 70299, 70300, 70301, 70302 were selected from the ORFs having the nucleotide sequence represented by SEQ ID NO: 5786.

The probes having the nucleotide sequence represented by SEQ ID NOS: 70303, 70304, 70305, 70306, 70307, 70308, 70309, 70310, 70311, 70312, 70313 were selected from the ORFs having the nucleotide sequence represented by SEQ ID NO: 5787.

The probes having the nucleotide sequence represented by SEQ ID NOS: 70314, 70315, 70316, 70317, 70318, 70319, 70320, 70321, 70322, 70323, 70324 were selected from the ORFs having the nucleotide sequence represented by SEQ ID NO: 5788.

The probes having the nucleotide sequence represented by SEQ ID NOS: 70325, 70326, 70327, 70328, 70329, 70330, 70331, 70332, 70333, 70334, 70335 were selected from the ORFs having the nucleotide sequence represented by SEQ ID NO: 5789.

The probes having the nucleotide sequence represented by SEQ ID NOS: 70336, 70337, 70338, 70339, 70340, 70341, 70342, 70343, 70344, 70345, 70346 were selected from the ORFs having the nucleotide sequence represented by SEQ ID NO: 5790.

The probes having the nucleotide sequence represented by SEQ ID NOS: 70347, 70348, 70349, 70350, 70351, 70352, 70353, 70354, 70355, 70356, 70357 were selected from the ORFs having the nucleotide sequence represented by SEQ ID NO: 5791.

The probes having the nucleotide sequence represented by SEQ ID NOS: 70358, 70359, 70360, 70361, 70362, 70363, 70364, 70365, 70366, 70367, 70368 were selected from the ORFs having the nucleotide sequence represented by SEQ ID NO: 5792.

The probes having the nucleotide sequence represented by SEQ ID NOS: 70369, 70370, 70371, 70372, 70373, 70374, 70375, 70376, 70377, 70378, 70379 were selected from the ORFs having the nucleotide sequence represented by SEQ ID NO: 5793.

The probes having the nucleotide sequence represented by SEQ ID NOS: 70380, 70381, 70382, 70383, 70384, 70385, 70386, 70387, 70388, 70389, 70390 were selected from the ORFs having the nucleotide sequence represented by SEQ ID NO: 5794.

The probes having the nucleotide sequence represented by SEQ ID NOS: 70391, 70392, 70393, 70394, 70395, 70396, 70397, 70398, 70399, 70400, 70401 were selected from the ORFs having the nucleotide sequence represented by SEQ ID NO: 5795.

The probes having the nucleotide sequence represented by SEQ ID NOS: 70402, 70403, 70404, 70405, 70406, 70407, 70408, 70409, 70410, 70411, 70412 were selected from the ORFs having the nucleotide sequence represented by SEQ ID NO: 5796.

The probes having the nucleotide sequence represented by SEQ ID NOS: 70413, 70414, 70415, 70416, 70417, 70418, 70419, 70420, 70421, 70422, 70423 were selected from the ORFs having the nucleotide sequence represented by SEQ ID NO: 5797.

The probes having the nucleotide sequence represented by SEQ ID NOS: 70424, 70425, 70426, 70427, 70428, 70429, 70430, 70431, 70432, 70433, 70434, 70435, 70436, 70437, 70438, 70439, 70440, 70441, 70442, 70443, 70444, 70445 were selected from the ORFs having the nucleotide sequence represented by SEQ ID NO: 5798.

The probes having the nucleotide sequence represented by SEQ ID NOS: 70446, 70447, 70448, 70449, 70450, 70451, 70452, 70453, 70454, 70455, 70456 were selected from the ORFs having the nucleotide sequence represented by SEQ ID NO: 5799.

The probes having the nucleotide sequence represented by SEQ ID NOS: 70457, 70458, 70459, 70460, 70461, 70462, 70463, 70464, 70465, 70466, 70467 were selected from the ORFs having the nucleotide sequence represented by SEQ ID NO: 5800.

The probes having the nucleotide sequence represented by SEQ ID NOS: 70468, 70469, 70470, 70471, 70472, 70473, 70474, 70475, 70476, 70477, 70478 were selected from the ORFs having the nucleotide sequence represented by SEQ ID NO: 5801.

The probes having the nucleotide sequence represented by SEQ ID NOS: 70479, 70480, 70481, 70482, 70483, 70484, 70485, 7.0486, 70487, 70488, 70489 were selected from the ORFs having the nucleotide sequence represented by SEQ ID NO: 5802.

The probes having the nucleotide sequence represented by SEQ ID NOS: 70490, 70491, 70492, 70493, 70494, 70495, 70496, 70497, 70498, 70499, 70500 were selected from the ORFs having the nucleotide sequence represented by SEQ ID NO: 5803.

The probes having the nucleotide sequence represented by SEQ ID NOS: 70501, 70502, 70503, 70504, 70505, 70506, 70507, 70508, 70509, 70510, 70511 were selected from the ORFs having the nucleotide sequence represented by SEQ ID NO: 5804.

The probes having the nucleotide sequence represented by SEQ ID NOS: 70512, 70513, 70514, 70515, 70516, 70517, 70518, 70519, 70520, 70521, 70522 were selected from the ORFs having the nucleotide sequence represented by SEQ ID NO: 5805.

The probes having the nucleotide sequence represented by SEQ ID NOS: 70523, 70524, 70525, 70526, 70527, 70528, 70529, 70530, 70531, 70532, 70533 were selected from the ORFs having the nucleotide sequence represented by SEQ ID NO: 5806.

The probes having the nucleotide sequence represented by SEQ ID NOS: 70534, 70535, 70536, 70537, 70538, 70539, 70540, 70541, 70542, 70543, 70544, 70545, 70546, 70547, 70548, 70549, 70550, 70551, 70552, 70553, 70554, 70555 were selected from the ORFs having the nucleotide sequence represented by SEQ ID NO: 5807.

The probes having the nucleotide sequence represented by SEQ ID NOS: 70556, 70557, 70558, 70559, 70560, 70561, 70562, 70563, 70564, 70565, 70566 were selected from the ORFs having the nucleotide sequence represented by SEQ ID NO: 5808.

The probes having the nucleotide sequence represented by SEQ ID NOS: 70567, 70568, 70569, 70570, 70571, 70572, 70573, 70574, 70575, 70576, 70577 were selected from the ORFs having the nucleotide sequence represented by SEQ ID NO: 5809.

The probes having the nucleotide sequence represented by SEQ ID NOS: 70578, 70579, 70580, 70581, 70582, 70583, 70584, 70585, 70586, 70587, 70588 were selected from the ORFs having the nucleotide sequence represented by SEQ ID NO: 5810.

The probes having the nucleotide sequence represented by SEQ ID NOS: 70589, 70590, 70591, 70592, 70593, 70594, 70595, 70596, 70597, 70598, 70599 were selected from the ORFs having the nucleotide sequence represented by SEQ ID NO: 5811.

The probes having the nucleotide sequence represented by SEQ ID NOS: 70600, 70601, 70602, 70603, 70604, 70605,

70606, 70607, 70608, 70609, 70610 were selected from the ORFs having the nucleotide sequence represented by SEQ ID NO: 5812.

The probes having the nucleotide sequence represented by SEQ ID NOS: 70611, 70612, 70613, 70614, 70615, 70616, 70617, 70618, 70619, 70620, 70621 were selected from the ORFs having the nucleotide sequence represented by SEQ ID NO: 5813.

The probes having the nucleotide sequence represented by SEQ ID NOS: 70622, 70623, 70624, 70625, 70626, 70627, 70628, 70629, 70630, 70631, 70632 were selected from the ORFs having the nucleotide sequence represented by SEQ ID NO: 5814.

The probes having the nucleotide sequence represented by SEQ ID NOS: 70633, 70634, 70635, 70636, 70637, 70638, 70639, 70640, 70641, 70642, 70643 were selected from the ORFs having the nucleotide sequence represented by SEQ ID NO: 5815.

The probes having the nucleotide sequence represented by SEQ ID NOS: 70644, 70645, 70646, 70647, 70648, 70649, 70650, 70651, 70652, 70653, 70654 were selected from the ORFs having the nucleotide sequence represented by SEQ ID NO: 5816.

The probes having the nucleotide sequence represented by SEQ ID NOS: 70655, 70656, 70657, 70658, 70659, 70660, 70661, 70662, 70663, 70664, 70665 were selected from the ORFs having the nucleotide sequence represented by SEQ ID NO: 5817.

The probes having the nucleotide sequence represented by SEQ ID NOS: 70666, 70667, 70668, 70669, 70670, 70671, 70672, 70673, 70674, 70675, 70676 were selected from the ORFs having the nucleotide sequence represented by SEQ ID NO: 5818.

The probes having the nucleotide sequence represented by SEQ ID NOS: 70677, 70678, 70679, 70680, 70681, 70682, 70683, 70684, 70685, 70686, 70687 were selected from the ORFs having the nucleotide sequence represented by SEQ ID NO: 5819.

The probes having the nucleotide sequence represented by SEQ ID NOS: 70688, 70689, 70690, 70691, 70692, 70693, 70694, 70695, 70696, 70697, 70698 were selected from the ORFs having the nucleotide sequence represented by SEQ ID NO: 5820.

The probes having the nucleotide sequence represented by SEQ ID NOS: 70699, 70700, 70701, 70702, 70703, 70704, 70705, 70706, 70707, 70708, 70709 were selected from the ORFs having the nucleotide sequence represented by SEQ ID NO: 5821.

The probes having the nucleotide sequence represented by SEQ ID NOS: 70710, 70711, 70712, 70713, 70714, 70715, 70716, 70717, 70718, 70719, 70720 were selected from the ORFs having the nucleotide sequence represented by SEQ ID NO: 5822.

The probes having the nucleotide sequence represented by SEQ ID NOS: 70721, 70722, 70723, 70724, 70725, 70726, 70727, 70728, 70729, 70730, 70731 were selected from the ORFs having the nucleotide sequence represented by SEQ ID NO: 5823.

The probes having the nucleotide sequence represented by SEQ ID NOS: 70732, 70733, 70734, 70735, 70736, 70737, 70738, 70739, 70740, 70741, 70742 were selected from the ORFs having the nucleotide sequence represented by SEQ ID NO: 5824.

The probes having the nucleotide sequence represented by SEQ ID NOS: 70743, 70744, 70745, 70746, 70747, 70748, 70749, 70750, 70751, 70752, 70753 were selected from the ORFs having the nucleotide sequence represented by SEQ ID NO: 5825.

The probes having the nucleotide sequence represented by SEQ ID NOS: 70754, 70755, 70756, 70757, 70758, 70759, 70760, 70761, 70762, 70763, 70764 were selected from the ORFs having the nucleotide sequence represented by SEQ ID NO: 5826.

The probes having the nucleotide sequence represented by SEQ ID NOS: 70765, 70766, 70767, 70768, 70769, 70770, 70771, 70772, 70773, 70774, 70775 were selected from the ORFs having the nucleotide sequence represented by SEQ ID NO: 5827.

The probes having the nucleotide sequence represented by SEQ ID NOS: 70776, 70777, 70778, 70779, 70780, 70781, 70782, 70783, 70784, 70785, 70786 were selected from the ORFs having the nucleotide sequence represented by SEQ ID NO: 5828.

The probes having the nucleotide sequence represented by SEQ ID NOS: 70787, 70788, 70789, 70790, 70791, 70792, 70793, 70794, 70795, 70796, 70797 were selected from the ORFs having the nucleotide sequence represented by SEQ ID NO: 5829.

The probes having the nucleotide sequence represented by SEQ ID NOS: 70798, 70799, 70800, 70801, 70802, 70803, 70804, 70805, 70806, 70807, 70808 were selected from the ORFs having the nucleotide sequence represented by SEQ ID NO: 5830.

The probes having the nucleotide sequence represented by SEQ ID NOS: 70809, 70810, 70811, 70812, 70813, 70814, 70815, 70816, 70817, 70818, 70819 were selected from the ORFs having the nucleotide sequence represented by SEQ ID NO: 5831.

The probes having the nucleotide sequence represented by SEQ ID NOS: 70820, 70821, 70822, 70823, 70824, 70825, 70826, 70827, 70828, 70829, 70830 were selected from the ORFs having the nucleotide sequence represented by SEQ ID NO: 5832.

The probes having the nucleotide sequence represented by SEQ ID NOS: 70831, 70832, 70833, 70834, 70835, 70836, 70837, 70838, 70839, 70840, 70841 were selected from the ORFs having the nucleotide sequence represented by SEQ ID NO: 5833.

The probes having the nucleotide sequence represented by SEQ ID NOS: 70842, 70843, 70844, 70845, 70846, 70847, 70848, 70849, 70850, 70851, 70852 were selected from the ORFs having the nucleotide sequence represented by SEQ ID NO: 5834.

The probes having the nucleotide sequence represented by SEQ ID NOS: 70853, 70854, 70855, 70856, 70857, 70858, 70859, 70860, 70861, 70862, 70863 were selected from the ORFs having the nucleotide sequence represented by SEQ ID NO: 5835.

The probes having-the nucleotide sequence represented by SEQ ID NOS: 70864, 70865, 70866, 70867, 70868, 70869, 70870, 70871, 70872, 70873, 70874 were selected from the ORFs having the nucleotide sequence represented by SEQ ID NO: 5836.

The probes having the nucleotide sequence represented by SEQ ID NOS: 70875, 70876, 70877, 70878, 70879, 70880, 70881, 70882, 70883, 70884, 70885 were selected from the ORFs having the nucleotide sequence represented by SEQ ID NO: 5837.

The probes having the nucleotide sequence represented by SEQ ID NOS: 70886, 70887, 70888, 70889, 70890, 70891, 70892, 70893, 70894, 70895, 70896 were selected from the ORFs having the nucleotide sequence represented by SEQ ID NO: 5838.

The probes having the nucleotide sequence represented by SEQ ID NOS: 70897, 70898, 70899, 70900, 70901, 70902, 70903, 70904, 70905, 70906, 70907 were selected from the ORFs having the nucleotide sequence represented by SEQ ID NO: 5839.

The probes having the nucleotide sequence represented by SEQ ID NOS: 70908, 70909, 70910, 70911, 70912, 70913, 70914, 70915, 70916, 70917, 70918 were selected from the ORFs having the nucleotide sequence represented by SEQ ID NO: 5840.

The probes having the nucleotide sequence represented by SEQ ID NOS: 70919, 70920, 70921, 70922, 70923, 70924, 70925, 70926, 70927, 70928, 70929 were selected from the ORFs having the nucleotide sequence represented by SEQ ID NO: 5841.

The probes having the nucleotide sequence represented by SEQ ID NOS: 70930, 70931, 70932, 70933, 70934, 70935, 70936, 70937, 70938, 70939, 70940 were selected from the ORFs having the nucleotide sequence represented by SEQ ID NO: 5842.

The probes having the nucleotide sequence represented by SEQ ID NOS: 70941, 70942, 70943, 70944, 70945, 70946, 70947, 70948, 70949, 70950, 70951 were selected from the ORFs having the nucleotide sequence represented by SEQ ID NO: 5843.

The probes having the nucleotide sequence represented by SEQ ID NOS: 70952, 70953, 70954, 70955, 70956, 70957, 70958, 70959, 70960, 70961, 70962 were selected from the ORFs having the nucleotide sequence represented by SEQ ID NO: 5844.

The probes having the nucleotide sequence represented by SEQ ID NOS: 70963, 70964, 70965, 70966, 70967, 70968, 70969, 70970, 70971, 70972, 70973 were selected from the ORFs having the nucleotide sequence represented by SEQ ID NO: 5845.

The probes having the nucleotide sequence represented by SEQ ID NOS: 70974, 70975, 70976, 70977, 70978, 70979, 70980, 70981, 70982, 70983, 70984 were selected from the ORFs having the nucleotide sequence represented by SEQ ID NO: 5846.

The probes having the nucleotide sequence represented by SEQ ID NOS: 70985, 70986, 70987, 70988, 70989, 70990, 70991, 70992, 70993, 70994, 70995 were selected from the ORFs having the nucleotide sequence represented by SEQ ID NO: 5847.

The probes having the nucleotide sequence represented by SEQ ID NOS: 70996, 70997, 70998, 70999, 71000, 71001, 71002, 71003, 71004, 71005, 71006 were selected from the ORFs having the nucleotide sequence represented by SEQ ID NO: 5848.

The probes having the nucleotide sequence represented by SEQ ID NOS: 71007, 71008, 71009, 71010, 71011, 71012, 71013, 71014, 71015, 71016, 71017 were selected from the ORFs having the nucleotide sequence represented by SEQ ID NO: 5849.

The probes having the nucleotide sequence represented by SEQ ID NOS: 71018, 71019, 71020, 71021, 71022, 71023, 71024, 71025, 71026, 71027, 71028 were selected from the ORFs having the nucleotide sequence represented by SEQ ID NO: 5850.

The probes having the nucleotide sequence represented by SEQ ID NOS: 71029, 71030, 71031, 71032, 71033, 71034, 71035, 71036, 71037, 71038, 71039 were selected from the ORFs having the nucleotide sequence represented by SEQ ID NO: 5851.

The probes having the nucleotide sequence represented by SEQ ID NOS: 71040, 71041, 71042, 71043, 71044, 71045, 71046, 71047, 71048, 71049, 71050 were selected from the ORFs having the nucleotide sequence represented by SEQ ID NO: 5852.

The probes having the nucleotide sequence represented by SEQ ID NOS: 71051, 71052, 71053, 71054, 71055, 71056, 71057, 71058, 71059, 71060, 71061 were selected from the ORFs having the nucleotide sequence represented by SEQ ID NO: 5853.

The probes having the nucleotide sequence represented by SEQ ID NOS: 71062, 71063, 71064, 71065, 71066, 71067, 71068, 71069, 71070, 71071, 71072 were selected from the ORFs having the nucleotide sequence represented by SEQ ID NO: 5854.

The probes having the nucleotide sequence represented by SEQ ID NOS: 71073, 71074, 71075, 71076, 71077, 71078, 71079, 71080, 71081, 71082, 71083 were selected from the ORFs having the nucleotide sequence represented by SEQ ID NO: 5855.

The probes having the nucleotide sequence represented by SEQ ID NOS: 71084, 71085, 71086, 71087, 71088, 71089, 71090, 71091, 71092, 71093, 71094 were selected from the ORFs having the nucleotide sequence represented by SEQ ID NO: 5856.

The probes having the nucleotide sequence represented by SEQ ID NOS: 71095, 71096, 71097, 71098, 71099, 71100, 71101, 71102, 71103, 71104, 71105 were selected from the ORFs having the nucleotide sequence represented by SEQ ID NO: 5857.

The probes having the nucleotide sequence represented by SEQ ID NOS: 71106, 71107, 71108, 71109, 71110, 71111, 71112, 71113, 71114, 71115, 71116 were selected from the ORFs having the nucleotide sequence represented by SEQ ID NO: 5858.

The probes having the nucleotide sequence represented by SEQ ID NOS: 71117, 71118, 71119, 71120, 71121, 71122, 71123, 71124, 71125, 71126, 71127 were selected from the ORFs having the nucleotide sequence represented by SEQ ID NO: 5859.

The probes having the nucleotide sequence represented by SEQ ID NOS: 71128, 71129, 71130, 71131, 71132, 71133, 71134, 71135, 71136, 71137, 71138 were selected from the ORFs having the nucleotide sequence represented by SEQ ID NO: 5860.

The probes having the nucleotide sequence represented by SEQ ID NOS: 71139, 71140, 71141, 71142, 71143, 71144, 71145, 71146, 71147, 71148, 71149 were selected from the ORFs having the nucleotide sequence represented by SEQ ID NO: 5861.

The probes having the nucleotide sequence represented by SEQ ID NOS: 71150, 71151, 71152, 71153, 71154, 71155,

71156, 71157, 71158, 71159, 71160 were selected from the ORFs having the nucleotide sequence represented by SEQ ID NO: 5862.

The probes having the nucleotide sequence represented by SEQ ID NOS: 71161, 71162, 71163, 71164, 71165, 71166, 71167, 71168, 71169, 71170, 71171 were selected from the ORFs having the nucleotide sequence represented by SEQ ID NO: 5863.

The probes having the nucleotide sequence represented by SEQ ID NOS: 71172, 71173, 71174, 71175, 71176, 71177, 71178, 71179, 71180, 71181, 71182 were selected from the ORFs having the nucleotide sequence represented by SEQ ID NO: 5864.

The probes having the nucleotide sequence represented by SEQ ID NOS: 71183, 71184, 71185, 71186, 71187, 71188, 71189, 71190, 71191, 71192, 71193 were selected from the ORFs having the nucleotide sequence represented by SEQ ID NO: 5865.

The probes having the nucleotide sequence represented by SEQ ID NOS: 71194, 71195, 71196, 71197, 71198, 71199, 71200, 71201, 71202, 71203, 71204 were selected from the ORFs having the nucleotide sequence represented by SEQ ID NO: 5866.

The probes having the nucleotide sequence represented by SEQ ID NOS: 71205, 71206, 71207, 71208, 71209, 71210, 71211, 71212, 71213, 71214, 71215 were selected from the ORFs having the nucleotide sequence represented by SEQ ID NO: 5867.

The probes having the nucleotide sequence represented by SEQ ID NOS: 71216, 71217, 71218, 71219, 71220, 71221, 71222, 71223, 71224, 71225, 71226 were selected from the ORFs having the nucleotide sequence represented by SEQ ID NO: 5868.

The probes having the nucleotide sequence represented by SEQ ID NOS: 71227, 71228, 71229, 71230, 71231, 71232, 71233, 71234, 71235, 71236, 71237 were selected from the ORFs having the nucleotide sequence represented by SEQ ID NO: 5869.

The probes having the nucleotide sequence represented by SEQ ID NOS: 71238, 71239, 71240, 71241, 71242, 71243, 71244, 71245, 71246, 71247, 71248 were selected from the ORFs having the nucleotide sequence represented by SEQ ID NO: 5870.

The probes having the nucleotide sequence represented by SEQ ID NOS: 71249, 71250, 71251, 71252, 71253, 71254, 71255, 71256, 71257, 71258, 71259 were selected from the ORFs having the nucleotide sequence represented by SEQ ID NO: 5871.

The probes having the nucleotide sequence represented by SEQ ID NOS: 71260, 71261, 71262, 71263, 71264, 71265, 71266, 71267, 71268, 71269, 71270 were selected from the ORFs having the nucleotide sequence represented by SEQ ID NO: 5872.

The probes having the nucleotide sequence represented by SEQ ID NOS: 71271, 71272, 71273, 71274, 71275, 71276, 71277, 71278, 71279, 71280, 71281 were selected from the ORFs having the nucleotide sequence represented by SEQ ID NO: 5873.

The probes having the nucleotide sequence represented by SEQ ID NOS: 71282, 71283, 71284, 71285, 71286, 71287, 71288, 71289, 71290, 71291, 71292 were selected from the ORFs having the nucleotide sequence represented by SEQ ID NO: 5874.

The probes having the nucleotide sequence represented by SEQ ID NOS: 71293, 71294, 71295, 71296, 71297, 71298, 71299, 71300, 71301, 71302, 71303 were selected from the ORFs having the nucleotide sequence represented by SEQ ID NO: 5875.

The probes having the nucleotide sequence represented by SEQ ID NOS: 71304, 71305, 71306, 71307, 71308, 71309, 71310, 71311, 71312, 71313, 71314 were selected from the ORFs having the nucleotide sequence represented by SEQ ID NO: 5876.

The probes having the nucleotide sequence represented by SEQ ID NOS: 71315, 71316, 71317, 71318, 71319, 71320, 71321, 71322, 71323, 71324, 71325 were selected from the ORFs having the nucleotide sequence represented by SEQ ID NO: 5877.

The probes having the nucleotide sequence represented by SEQ ID NOS: 71326, 71327, 71328, 71329, 71330, 71331, 71332, 71333, 71334, 71335, 71336 were selected from the ORFs having the nucleotide sequence represented by SEQ ID NO: 5878.

The probes having the nucleotide sequence represented by SEQ ID NOS: 71337, 71338, 71339, 71340, 71341, 71342, 71343, 71344, 71345, 71346, 71347 were selected from the ORFs having the nucleotide sequence represented by SEQ ID NO: 5879.

The probes having the nucleotide sequence represented by SEQ ID NOS: 71348, 71349, 71350, 71351, 71352, 71353, 71354, 71355, 71356, 71357, 71358 were selected from the ORFs having the nucleotide sequence represented by SEQ ID NO: 5880.

The probes having the nucleotide sequence represented by SEQ ID NOS: 71359, 71360, 71361, 71362, 71363, 71364, 71365, 71366, 71367, 71368, 71369 were selected from the ORFs having the nucleotide sequence represented by SEQ ID NO: 5881.

The probes having the nucleotide sequence represented by SEQ ID NOS: 71370, 71371, 71372, 71373, 71374, 71375, 71376, 71377, 71378, 71379, 71380 were selected from the ORFs having the nucleotide sequence represented by SEQ ID NO: 5882.

The probes having the nucleotide sequence represented by SEQ ID NOS: 71381, 71382, 71383, 71384, 71385, 71386, 71387, 71388, 71389, 71390, 71391 were selected from the ORFs having the nucleotide sequence represented by SEQ ID NO: 5883.

The probes having the nucleotide sequence represented by SEQ ID NOS: 71392, 71393, 71394, 71395, 71396, 71397, 71398, 71399, 71400, 71401, 71402 were selected from the ORFs having the nucleotide sequence represented by SEQ ID NO: 5884.

The probes having the nucleotide sequence represented by SEQ ID NOS: 71403, 71404, 71405, 71406, 71407, 71408, 71409, 71410, 71411, 71412, 71413 were selected from the ORFs having the nucleotide sequence represented by SEQ ID NO: 5885.

The probes having the nucleotide sequence represented by SEQ ID NOS: 71414, 71415, 71416, 71417, 71418, 71419, 71420, 71421, 71422, 71423, 71424 were selected from the ORFs having the nucleotide sequence represented by SEQ ID NO: 5886.

The probes having the nucleotide sequence represented by SEQ ID NOS: 71425, 71426, 71427, 71428, 71429, 71430, 71431, 71432, 71433, 71434, 71435 were selected from the ORFs having the nucleotide sequence represented by SEQ ID NO: 5887.

The probes having the nucleotide sequence represented by SEQ ID NOS: 71436, 71437, 71438, 71439, 71440, 71441, 71442, 71443, 71444, 71445, 71446 were selected from the ORFs having the nucleotide sequence represented by SEQ ID NO: 5888.

The probes having the nucleotide sequence represented by SEQ ID NOS: 71447, 71448, 71449, 71450, 71451, 71452, 71453, 71454, 71455, 71456, 71457 were selected from the ORFs having the nucleotide sequence represented by SEQ ID NO: 5889.

The probes having the nucleotide sequence represented by SEQ ID NOS: 71458, 71459, 71460, 71461, 71462, 71463, 71464, 71465, 71466, 71467, 71468 were selected from the ORFs having the nucleotide sequence represented by SEQ ID NO: 5890.

The probes having the nucleotide sequence represented by SEQ ID NOS: 71469, 71470, 71471, 71472, 71473, 71474, 71475, 71476, 71477, 71478, 71479 were selected from the ORFs having the nucleotide sequence represented by SEQ ID NO: 5891.

The probes having the nucleotide sequence represented by SEQ ID NOS: 71480, 71481, 71482, 71483, 71484, 71485, 71486, 71487, 71488, 71489, 71490 were selected from the ORFs having the nucleotide sequence represented by SEQ ID NO: 5892.

The probes having the nucleotide sequence represented by SEQ ID NOS: 71491, 71492, 71493, 71494, 71495, 71496, 71497, 71498, 71499, 71500, 71501 were selected from the ORFs having the nucleotide sequence represented by SEQ ID NO: 5893.

The probes having the nucleotide sequence represented by SEQ ID NOS: 71502, 71503, 71504, 71505, 71506, 71507, 71508, 71509, 71510, 71511, 71512 were selected from the ORFs having the nucleotide sequence represented by SEQ ID NO: 5894.

The probes having the nucleotide sequence represented by SEQ ID NOS: 71513, 71514, 71515, 71516, 71517, 71518, 71519, 71520, 71521, 71522, 71523 were selected from the ORFs having the nucleotide sequence represented by SEQ ID NO: 5895.

The probes having the nucleotide sequence represented by SEQ ID NOS: 71524, 71525, 71526, 71527, 71528, 71529, 71530, 71531, 71532, 71533, 71534 were selected from the ORFs having the nucleotide sequence represented by SEQ ID NO: 5896.

The probes having the nucleotide sequence represented by SEQ ID NOS: 71535, 71536, 71537, 71538, 71539, 71540, 71541, 71542, 71543, 71544, 71545 were selected from the ORFs having the nucleotide sequence represented by SEQ ID NO: 5897.

The probes having the nucleotide sequence represented by SEQ ID NOS: 71546, 71547, 71548, 71549, 71550, 71551, 71552, 71553, 71554, 71555, 71556 were selected from the ORFs having the nucleotide sequence represented by SEQ ID NO: 5898.

The probes having the nucleotide sequence represented by SEQ ID NOS: 71557, 71558, 71559, 71560, 71561, 71562, 71563, 71564, 71565, 71566, 71567 were selected from the ORFs having the nucleotide sequence represented by SEQ ID NO: 5899.

The probes having the nucleotide sequence represented by SEQ ID NOS: 71568, 71569, 71570, 71571, 71572, 71573, 71574, 71575, 71576, 71577, 71578 were selected from the ORFs having the nucleotide sequence represented by SEQ ID NO: 5900.

The probes having the nucleotide sequence represented by SEQ ID NOS: 71579, 71580, 71581, 71582, 71583, 71584, 71585, 71586, 71587, 71588, 71589 were selected from the ORFs having the nucleotide sequence represented by SEQ ID NO: 5901.

The probes having the nucleotide sequence represented by SEQ ID NOS: 71590, 71591, 71592, 71593, 71594, 71595, 71596, 71597, 71598, 71599, 71600 were selected from the ORFs having the nucleotide sequence represented by SEQ ID NO: 5902.

The probes having the nucleotide sequence represented by SEQ ID NOS: 71601, 71602, 71603, 71604, 71605, 71606, 71607, 71608, 71609, 71610, 71611 were selected from the ORFs having the nucleotide sequence represented by SEQ ID NO: 5903.

The probes having the nucleotide sequence represented by SEQ ID NOS: 71612, 71613, 71614, 71615, 71616, 71617, 71618, 71619, 71620, 71621, 71622 were selected from the ORFs having the nucleotide sequence represented by SEQ ID NO: 5904.

The probes having the nucleotide sequence represented by SEQ ID NOS: 71623, 71624, 71625, 71626, 71627, 71628, 71629, 71630, 71631, 71632, 71633 were selected from the ORFs having the nucleotide sequence represented by SEQ ID NO: 5905.

The probes having the nucleotide sequence represented by SEQ ID NOS: 71634, 71635, 71636, 71637, 71638, 71639, 71640, 71641, 71642, 71643, 71644 were selected from the ORFs having the nucleotide sequence represented by SEQ ID NO: 5906.

The probes having the nucleotide sequence represented by SEQ ID NOS: 71645, 71646, 71647, 71648, 71649, 71650, 71651, 71652, 71653, 71654, 71655 were selected from the ORFs having the nucleotide sequence represented by SEQ ID NO: 5907.

The probes having the nucleotide sequence represented by SEQ ID NOS: 71656, 71657, 71658, 71659, 71660, 71661, 71662, 71663, 71664, 71665, 71666 were selected from the ORFs having the nucleotide sequence represented by SEQ ID NO: 5908.

The probes having the nucleotide sequence represented by SEQ ID NOS: 71667, 71668, 71669, 71670, 71671, 71672, 71673, 71674, 71675, 71676, 71677 were selected from the ORFs having the nucleotide sequence represented by SEQ ID NO: 5909.

The probes having the nucleotide sequence represented by SEQ ID NOS: 71678, 71679, 71680, 71681, 71682, 71683, 71684, 71685, 71686, 71687, 71688 were selected from the ORFs having the nucleotide sequence represented by SEQ ID NO: 5910.

The probes having the nucleotide sequence represented by SEQ ID NOS: 71689, 71690, 71691, 71692, 71693, 71694, 71695, 71696, 71697, 71698, 71699 were selected from the ORFs having the nucleotide sequence represented by SEQ ID NO: 5911.

The probes having the nucleotide sequence represented by SEQ ID NOS: 71700, 71701, 71702, 71703, 71704, 71705, 71706, 71707, 71708, 71709, 71710 were selected from the ORFs having the nucleotide sequence represented by SEQ ID NO: 5912.

The probes having the nucleotide sequence represented by SEQ ID NOS: 71711, 71712, 71713, 71714, 71715, 71716, 71717, 71718, 71719, 71720, 71721 were selected from the ORFs having the nucleotide sequence represented by SEQ ID NO: 5913.

The probes having the nucleotide sequence represented by SEQ ID NOS: 71722, 71723, 71724, 71725, 71726, 71727, 71728, 71729, 71730, 71731, 71732 were selected from the ORFs having the nucleotide sequence represented by SEQ ID NO: 5914.

The probes having the nucleotide sequence represented by SEQ ID NOS: 71733, 71734, 71735, 71736, 71737, 71738, 71739, 71740, 71741, 71742, 71743 were selected from the ORFs having the nucleotide sequence represented by SEQ ID NO: 5915.

The probes having the nucleotide sequence represented by SEQ ID NOS: 71744, 71745, 71746, 71747, 71748, 71749, 71750, 71751, 71752, 71753, 71754 were selected from the ORFs having the nucleotide sequence represented by SEQ ID NO: 5916.

The probes having the nucleotide sequence represented by SEQ ID NOS: 71755, 71756, 71757, 71758, 71759, 71760, 71761, 71762, 71763, 71764, 71765 were selected from the ORFs having the nucleotide sequence represented by SEQ ID NO: 5917.

The probes having the nucleotide sequence represented by SEQ ID NOS: 71766, 71767, 71768, 71769, 71770, 71771, 71772, 71773, 71774, 71775, 71776 were selected from the ORFs having the nucleotide sequence represented by SEQ ID NO: 5918.

The probes having the nucleotide sequence represented by SEQ ID NOS: 71777, 71778, 71779, 71780, 71781, 71782, 71783, 71784, 71785, 71786, 71787 were selected from the ORFs having the nucleotide sequence represented by SEQ ID NO: 5919.

The probes having the nucleotide sequence represented by SEQ ID NOS: 71788, 71789, 71790, 71791, 71792, 71793, 71794, 71795, 71796, 71797, 71798 were selected from the ORFs having the nucleotide sequence represented by SEQ ID NO: 5920.

The probes having the nucleotide sequence represented by SEQ ID NOS: 71799, 71800, 71801, 71802, 71803, 71804, 71805, 71806, 71807, 71808, 71809 were selected from the ORFs having the nucleotide sequence represented by SEQ ID NO: 5921.

The probes having the nucleotide sequence represented by SEQ ID NOS: 71810, 71811, 71812, 71813, 71814, 71815, 71816, 71817, 71818, 71819, 71820 were selected from the ORFs having the nucleotide sequence represented by SEQ ID NO: 5922.

The probes having the nucleotide sequence represented by SEQ ID NOS: 71821, 71822, 71823, 71824, 71825, 71826, 71827, 71828, 71829, 71830, 71831 were selected from the ORFs having the nucleotide sequence represented by SEQ ID NO: 5923.

The probes having the nucleotide sequence represented by SEQ ID NOS: 71832, 71833, 71834, 71835, 71836, 71837, 71838, 71839, 71840, 71841, 71842 were selected from the ORFs having the nucleotide sequence represented by SEQ ID NO: 5924.

The probes having the nucleotide sequence represented by SEQ ID NOS: 71843, 71844, 71845, 71846, 71847, 71848, 71849, 71850, 71851, 71852, 71853 were selected from the ORFs having the nucleotide sequence represented by SEQ ID NO: 5925.

The probes having the nucleotide sequence represented by SEQ ID NOS: 71854, 71855, 71856, 71857, 71858, 71859, 71860, 71861, 71862, 71863, 71864 were selected from the ORFs having the nucleotide sequence represented by SEQ ID NO: 5926.

The probes having the nucleotide sequence represented by SEQ ID NOS: 71865, 71866, 71867, 71868, 71869, 71870, 71871, 71872, 71873, 71874, 71875 were selected from the ORFs having the nucleotide sequence represented by SEQ ID NO: 5927.

The probes having the nucleotide sequence represented by SEQ ID NOS: 71876, 71877, 71878, 71879, 71880, 71881, 71882, 71883, 71884, 71885, 71886 were selected from the ORFs having the nucleotide sequence represented by SEQ ID NO: 5928.

The probes having the nucleotide sequence represented by SEQ ID NOS: 71887, 71888, 71889, 71890, 71891, 71892, 71893, 71894, 71895, 71896, 71897 were selected from the ORFs having the nucleotide sequence represented by SEQ ID NO: 5929.

The probes having the nucleotide sequence represented by SEQ ID NOS: 71898, 71899, 71900, 71901, 71902, 71903, 71904, 71905, 71906, 71907, 71908 were selected from the ORFs having the nucleotide sequence represented by SEQ ID NO: 5930.

The probes having the nucleotide sequence represented by SEQ ID NOS: 71909, 71910, 71911, 71912, 71913, 71914, 71915, 71916, 71917, 71918, 71919 were selected from the ORFs having the nucleotide sequence represented by SEQ ID NO: 5931.

The probes having the nucleotide sequence represented by SEQ ID NOS: 71920, 71921, 71922, 71923, 71924, 71925, 71926, 71927, 71928, 71929, 71930 were selected from the ORFs having the nucleotide sequence represented by SEQ ID NO: 5932.

The probes having the nucleotide sequence represented by SEQ ID NOS: 71931, 71932, 71933, 71934, 71935, 71936, 71937, 71938, 71939, 71940, 71941 were selected from the ORFs having the nucleotide sequence represented by SEQ ID NO: 5933.

The probes having the nucleotide sequence represented by SEQ ID NOS: 71942, 71943, 71944, 71945, 71946, 71947, 71948, 71949, 71950, 71951, 71952 were selected from the ORFs having the nucleotide sequence represented by SEQ ID NO: 5934.

The probes having the nucleotide sequence represented by SEQ ID NOS: 71953, 71954, 71955, 71956, 71957, 71958, 71959, 71960, 71961, 71962, 71963 were selected from the ORFs having the nucleotide sequence represented by SEQ ID NO: 5935.

The probes having the nucleotide sequence represented by SEQ ID NOS: 71964, 71965, 71966, 71967, 71968, 71969, 71970, 71971, 71972, 71973, 71974 were selected from the ORFs having the nucleotide sequence represented by SEQ ID NO: 5936.

The probes having the nucleotide sequence represented by SEQ ID NOS: 71975, 71976, 71977, 71978, 71979, 71980, 71981, 71982, 71983, 71984, 71985 were selected from the ORFs having the nucleotide sequence represented by SEQ ID NO: 5937.

The probes having the nucleotide sequence represented by SEQ ID NOS: 71986, 71987, 71988, 71989, 71990, 71991, 71992, 71993, 71994, 719,95, 71996 were selected from the ORFs having the nucleotide sequence represented by SEQ ID NO: 5938.

The probes having the nucleotide sequence represented by SEQ ID NOS: 71997, 71998, 71999, 72000, 72001, 72002, 72003, 72004, 72005, 72006, 72007 were selected from the ORFs having the nucleotide sequence represented by SEQ ID NO: 5939.

The probes having the nucleotide sequence represented by SEQ ID NOS: 72008, 72009, 72010, 72011, 72012, 72013, 72014, 72015, 72016, 72017, 72018 were selected from the ORFs having the nucleotide sequence represented by SEQ ID NO: 5940.

The probes having the nucleotide sequence represented by SEQ ID NOS: 72019, 72020, 72021, 72022, 72023, 72024, 72025, 72026, 72027, 72028, 72029 were selected from the ORFs having the nucleotide sequence represented by SEQ ID NO: 5941.

The probes having the nucleotide sequence represented by SEQ ID NOS: 72030, 72031, 72032, 72033, 72034, 72035, 72036, 72037, 72038, 72039, 72040 were selected from the ORFs having the nucleotide sequence represented by SEQ ID NO: 5942.

The probes having the nucleotide sequence represented by SEQ ID NOS: 72041, 72042, 72043, 72044, 72045, 72046, 72047, 72048, 72049, 72050, 72051 were selected from the ORFs having the nucleotide sequence represented by SEQ ID NO: 5943.

The probes having the nucleotide sequence represented by SEQ ID NOS: 72052, 72053, 72054, 72055, 72056, 72057, 72058, 72059, 72060, 72061, 72062 were selected from the ORFs having the nucleotide sequence represented by SEQ ID NO: 5944.

The probes having the nucleotide sequence represented by SEQ ID NOS: 72063, 72064, 72065, 72066, 72067, 72068, 72069, 72070, 72071, 72072, 72073 were selected from the ORFs having the nucleotide sequence represented by SEQ ID NO: 5945.

The probes having the nucleotide sequence represented by SEQ ID NOS: 72074, 72075, 72076, 72077, 72078, 72079, 72080, 72081, 72082, 72083, 72084 were selected from the ORFs having the nucleotide sequence represented by SEQ ID NO: 5946.

The probes having the nucleotide sequence represented by SEQ ID NOS: 72085, 72086, 72087, 72088, 72089, 72090, 72091, 72092, 72093, 72094, 72095 were selected from the ORFs having the nucleotide sequence represented by SEQ ID NO: 5947.

The probes having the nucleotide sequence represented by SEQ ID NOS: 72096, 72097, 72098, 72099, 72100, 72101, 72102, 72103, 72104, 72105, 72106 were selected from the ORFs having the nucleotide sequence represented by SEQ ID NO: 5948.

The probes having the nucleotide sequence represented by SEQ ID NOS: 72107, 72108, 72109, 72110, 72111, 72112, 72113, 72114, 72115, 72116, 72117 were selected from the ORFs having the nucleotide sequence represented by SEQ ID NO: 5949.

The probes having the nucleotide sequence represented by SEQ ID NOS: 72118, 72119, 72120, 72121, 72122, 72123, 72124, 72125, 72126, 72127, 72128 were selected from the ORFs having the nucleotide sequence represented by SEQ ID NO: 5950.

The probes having the nucleotide sequence represented by SEQ ID NOS: 72129, 72130, 72131, 72132, 72133, 72134, 72135, 72136, 72137, 72138, 72139 were selected from the ORFs having the nucleotide sequence represented by SEQ ID NO: 5951.

The probes having the nucleotide sequence represented by SEQ ID NOS: 72140, 72141, 72142, 72143, 72144, 72145, 72146, 72147, 72148, 72149, 72150 were selected from the ORFs having the nucleotide sequence represented by SEQ ID NO: 5952.

The probes having the nucleotide sequence represented by SEQ ID NOS: 72151, 72152, 72153, 72154, 72155, 72156, 72157, 72158, 72159, 72160, 72161, 72162, 72163, 72164, 72165, 72166, 72167, 72168, 72169, 72170, 72171, 72172 were selected from the ORFs having the nucleotide sequence represented by SEQ ID NO: 5953.

The probes having the nucleotide sequence represented by SEQ ID NOS: 72173, 72174, 72175, 72176, 72177, 72178, 72179, 72180, 72181, 72182, 72183 were selected from the ORFs having the nucleotide sequence represented by SEQ ID NO: 5954.

The probes having the nucleotide sequence represented by SEQ ID NOS: 72184, 72185, 72186, 72187, 72188, 72189, 72190, 72191, 72192, 72193, 72194 were selected from the ORFs having the nucleotide sequence represented by SEQ ID NO: 5955.

The probes having the nucleotide sequence represented by SEQ ID NOS: 72195, 72196, 72197, 72198, 72199, 72200, 72201, 72202, 72203, 72204, 72205 were selected from the ORFs having the nucleotide sequence represented by SEQ ID NO: 5956.

The probes having the nucleotide sequence represented by SEQ ID NOS: 72206, 72207, 72208, 72209, 72210, 72211, 72212, 72213, 72214, 72215, 72216 were selected from the ORFs having the nucleotide sequence represented by SEQ ID NO: 5957.

The probes having the nucleotide sequence represented by SEQ ID NOS: 72217, 72218, 72219, 72220, 72221, 72222, 72223, 72224, 72225, 72226, 72227 were selected from the ORFs having the nucleotide sequence represented by SEQ ID NO: 5958.

The probes having the nucleotide sequence represented by SEQ ID NOS: 72228, 72229, 72230, 72231, 72232, 72233, 72234, 72235, 72236, 72237, 72238 were selected from the ORFs having the nucleotide sequence represented by SEQ ID NO: 5959.

The probes having the nucleotide sequence represented by SEQ ID NOS: 72239, 72240, 72241, 72242, 72243, 72244, 72245, 72246, 72247, 72248, 72249 were selected from the ORFs having the nucleotide sequence represented by SEQ ID NO: 5960.

The probes having the nucleotide sequence represented by SEQ ID NOS: 72250, 72251, 72252, 72253, 72254, 72255, 72256, 72257, 72258, 72259, 72260 were selected from the ORFs having the nucleotide sequence represented by SEQ ID NO: 5961.

The probes having the nucleotide sequence represented by SEQ ID NOS: 72261, 72262, 72263, 72264, 72265, 72266, 72267, 72268, 72269, 72270, 72271 were selected from the ORFs having the nucleotide sequence represented by SEQ ID NO: 5962.

The probes having the nucleotide sequence represented by SEQ ID NOS: 72272, 72273, 72274, 72275, 72276, 72277, 72278, 72279, 72280, 72281, 72282 were selected from the ORFs having the nucleotide sequence represented by SEQ ID NO: 5963.

The probes having the nucleotide sequence represented by SEQ ID NOS: 72283, 72284, 72285, 72286, 72287, 72288, 72289, 72290, 72291, 72292, 72293 were selected from the ORFs having the nucleotide sequence represented by SEQ ID NO: 5964.

The probes having the nucleotide sequence represented by SEQ ID NOS: 72294, 72295, 72296, 72297, 72298, 72299, 72300, 72301, 72302, 72303, 72304 were selected from the ORFs having the nucleotide sequence represented by SEQ ID NO: 5965.

The probes having the nucleotide sequence represented by SEQ ID NOS: 72305, 72306, 72307, 72308, 72309, 72310, 72311, 72312, 72313, 72314, 72315 were selected from the ORFs having the nucleotide sequence represented by SEQ ID NO: 5966.

The probes having the nucleotide sequence represented by SEQ ID NOS: 72316, 72317, 72318, 72319, 72320, 72321, 72322, 72323, 72324, 72325, 72326 were selected from the ORFs having the nucleotide sequence represented by SEQ ID NO: 5967.

The probes having the nucleotide sequence represented by SEQ ID NOS: 72327, 72328, 72329, 72330, 72331, 72332, 72333, 72334, 72335, 72336, 72337 were selected from the ORFs having the nucleotide sequence represented by SEQ ID NO: 5968.

The probes having the nucleotide sequence represented by SEQ ID NOS: 72338, 72339, 72340, 72341, 72342, 72343, 72344, 72345, 72346, 72347, 72348 were selected from the ORFs having the nucleotide sequence represented by SEQ ID NO: 5969.

The probes having the nucleotide sequence represented by SEQ ID NOS: 72349, 72350, 72351, 72352, 72353, 72354, 72355, 72356, 72357, 72358, 72359 were selected from the ORFs having the nucleotide sequence represented by SEQ ID NO: 5970.

The probes having the nucleotide sequence represented by SEQ ID NOS: 72360, 72361, 72362, 72363, 72364, 72365, 72366, 72367, 72368, 72369, 72370, 72371, 72372, 72373, 72374, 72375, 72376, 72377, 72378, 72379, 72380, 72381 were selected from the ORFs having the nucleotide sequence represented by SEQ ID NO: 5971.

The probes having the nucleotide sequence represented by SEQ ID NOS: 72382, 72383, 72384, 72385, 72386, 72387, 72388, 72389, 72390, 72391, 72392 were selected from the ORFs having the nucleotide sequence represented by SEQ ID NO: 5972.

The probes having the nucleotide sequence represented by SEQ ID NOS: 72393, 72394, 72395, 72396, 72397, 72398, 72399, 72400, 72401, 72402, 72403 were selected from the ORFs having the nucleotide sequence represented by SEQ ID NO: 5973.

The probes having the nucleotide sequence represented by SEQ ID NOS: 72404, 72405, 72406, 72407, 72408, 72409, 72410, 72411, 72412, 72413, 72414 were selected from the ORFs having the nucleotide sequence represented by SEQ ID NO: 5974.

The probes having the nucleotide sequence represented by SEQ ID NOS: 72415, 72416, 72417, 72418, 72419, 72420, 72421, 72422, 72423, 72424, 72425 were selected from the ORFs having the nucleotide sequence represented by SEQ ID NO: 5975.

The probes having the nucleotide sequence represented by SEQ ID NOS: 72426, 72427, 72428, 72429, 72430, 72431, 72432, 72433, 72434, 72435, 72436 were selected from the ORFs having the nucleotide sequence represented by SEQ ID NO: 5976.

The probes having the nucleotide sequence represented by SEQ ID NOS: 72437, 72438, 72439, 72440, 72441, 72442, 72443, 72444, 72445, 72446, 72447 were selected from the ORFs having the nucleotide sequence represented by SEQ ID NO: 5977.

The probes having the nucleotide sequence represented by SEQ ID NOS: 72448, 72449, 72450, 72451, 72452, 72453, 72454, 72455, 72456, 72457, 72458, 72459, 72460, 72461, 72462, 72463, 72464, 72465, 72466, 72467, 72468, 72469 were selected from the ORFs having the nucleotide sequence represented by SEQ ID NO: 5978.

The probes having the nucleotide sequence represented by SEQ ID NOS: 72470, 72471, 72472, 72473, 72474, 72475, 72476, 72477, 72478, 72479, 72480 were selected from the ORFs having the nucleotide sequence represented by SEQ ID NO: 5979.

The probes having the nucleotide sequence represented by SEQ ID NOS: 72481, 72482, 72483, 72484, 72485, 72486, 72487, 72488, 72489, 72490, 72491 were selected from the ORFs having the nucleotide sequence represented by SEQ ID NO: 5980.

The probes having the nucleotide sequence represented by SEQ ID NOS: 72492, 72493, 72494, 72495, 72496, 72497, 72498, 72499, 72500, 72501, 72502 were selected from the ORFs having the nucleotide sequence represented by SEQ ID NO: 5981.

The probes having the nucleotide sequence represented by SEQ ID NOS: 72503, 72504, 72505, 72506, 72507, 72508, 72509, 72510, 72511, 72512, 72513 were selected from the ORFs having the nucleotide sequence represented by SEQ ID NO: 5982.

The probes having the nucleotide sequence represented by SEQ ID NOS: 72514, 72515, 72516, 72517, 72518, 72519, 72520, 72521, 72522, 72523, 72524 were selected from the ORFs having the nucleotide sequence represented by SEQ ID NO: 5983.

The probes having the nucleotide sequence represented by SEQ ID NOS: 72525, 72526, 72527, 72528, 72529, 72530, 72531, 72532, 72533, 72534, 72535 were selected from the ORFs having the nucleotide sequence represented by SEQ ID NO: 5984.

The probes having the nucleotide sequence represented by SEQ ID NOS: 72536, 72537, 72538, 72539, 72540, 72541, 72542, 72543, 72544, 72545, 72546 were selected from the ORFs having the nucleotide sequence represented by SEQ ID NO: 5985.

The probes having the nucleotide sequence represented by SEQ ID NOS: 72547, 72548, 72549, 72550, 72551, 72552, 72553, 72554, 72555, 72556, 72557 were selected from the ORFs having the nucleotide sequence represented by SEQ ID NO: 5986.

The probes having the nucleotide sequence represented by SEQ ID NOS: 72558, 72559, 72560, 72561, 72562, 72563, 72564, 72565, 72566, 72567, 72568 were selected from the ORFs having the nucleotide sequence represented by SEQ ID NO: 5987.

The probes having the nucleotide sequence represented by SEQ ID NOS: 72569, 72570, 72571, 72572, 72573, 72574, 72575, 72576, 72577, 72578, 72579 were selected from the ORFs having the nucleotide sequence represented by SEQ ID NO: 5988.

The probes having the nucleotide sequence represented by SEQ ID NOS: 72580, 72581, 72582, 72583, 72584, 72585, 72586, 72587, 72588, 72589, 72590 were selected from the ORFs having the nucleotide sequence represented by SEQ ID NO: 5989.

The probes having the nucleotide sequence represented by SEQ ID NOS: 72591, 72592, 72593, 72594, 72595, 72596, 72597, 72598, 72599, 72600, 72601 were selected from the ORFs having the nucleotide sequence represented by SEQ ID NO: 5990.

The probes having the nucleotide sequence represented by SEQ ID NOS: 72602, 72603, 72604, 72605, 72606, 72607, 72608, 72609, 72610, 72611, 72612 were selected from the ORFs having the nucleotide sequence represented by SEQ ID NO: 5991.

The probes having the nucleotide sequence represented by SEQ ID NOS: 72613, 72614, 72615, 72616, 72617, 72618, 72619, 72620, 72621, 72622, 72623 were selected from the ORFs having the nucleotide sequence represented by SEQ ID NO: 5992.

The probes having the nucleotide sequence represented by SEQ ID NOS: 72624, 72625, 72626, 72627, 72628, 72629, 72630, 72631, 72632, 72633, 72634 were selected from the ORFs having the nucleotide sequence represented by SEQ ID NO: 5993.

The probes having the nucleotide sequence represented by SEQ ID NOS: 72635, 72636, 72637, 72638, 72639, 72640, 72641, 72642, 72643, 72644, 72645 were selected from the ORFs having the nucleotide sequence represented by SEQ ID NO: 5994.

The probes having the nucleotide sequence represented by SEQ ID NOS: 72646, 72647, 72648, 72649, 72650, 72651, 72652, 72653, 72654, 72655, 72656 were selected from the ORFs having the nucleotide sequence represented by SEQ ID NO: 5995.

The probes having the nucleotide sequence represented by SEQ ID NOS: 72657, 72658, 72659, 72660, 72661, 72662, 72663, 72664, 72665, 72666, 72667 were selected from the ORFs having the nucleotide sequence represented by SEQ ID NO: 5996.

The probes having the nucleotide sequence represented by SEQ ID NOS: 72668, 72669, 72670, 72671, 72672, 72673, 72674, 72675, 72676, 72677, 72678 were selected from the ORFs having the nucleotide sequence represented by SEQ ID NO: 5997.

The probes having the nucleotide sequence represented by SEQ ID NOS: 72679, 72680, 72681, 72682, 72683, 72684, 72685, 72686, 72687, 72688, 72689 were selected from the ORFs having the nucleotide sequence represented by SEQ ID NO: 5998.

The probes having the nucleotide sequence represented by SEQ ID NOS: 72690, 72691, 72692, 72693, 72694, 72695, 72696, 72697, 72698, 72699, 72700 were selected from the ORFs having the nucleotide sequence represented by SEQ ID NO: 5999.

The probes having the nucleotide sequence represented by SEQ ID NOS: 72701, 72702, 72703, 72704, 72705, 72706, 72707, 72708, 72709, 72710, 72711 were selected from the ORFs having the nucleotide sequence represented by SEQ ID NO: 6000.

The probes having the nucleotide sequence represented by SEQ ID NOS: 72712, 72713, 72714, 72715, 72716, 72717, 72718, 72719, 72720, 72721, 72722 were selected from the ORFs having the nucleotide sequence represented by SEQ ID NO: 6001.

The probes having the nucleotide sequence represented by SEQ ID NOS: 72723, 72724, 72725, 72726, 72727, 72728, 72729, 72730, 72731, 72732, 72733 were selected from the ORFs having the nucleotide sequence represented by SEQ ID NO: 6002.

The probes having the nucleotide sequence represented by SEQ ID NOS: 72734, 72735, 72736, 72737, 72738, 72739, 72740, 72741, 72742, 72743, 72744 were selected from the ORFs having the nucleotide sequence represented by SEQ ID NO: 6003.

The probes having the nucleotide sequence represented by SEQ ID NOS: 72745, 72746, 72747, 72748, 72749, 72750, 72751, 72752, 72753, 72754, 72755 were selected from the ORFs having the nucleotide sequence represented by SEQ ID NO: 6004.

The probes having the nucleotide sequence represented by SEQ ID NOS: 72756, 72757, 72758, 72759, 72760, 72761, 72762, 72763, 72764, 72765, 72766 were selected from the ORFs having the nucleotide sequence represented by SEQ ID NO: 6005.

The probes having the nucleotide sequence represented by SEQ ID NOS: 72767, 72768, 72769, 72770, 72771, 72772, 72773, 72774, 72775, 72776, 72777 were selected from the ORFs having the nucleotide sequence represented by SEQ ID NO: 6006.

The probes having the nucleotide sequence represented by SEQ ID NOS: 72778, 72779, 72780, 72781, 72782, 72783, 72784, 72785, 72786, 72787, 72788 were selected from the ORFs having the nucleotide sequence represented by SEQ ID NO: 6007.

The probes having the nucleotide sequence represented by SEQ ID NOS: 72789, 72790, 72791, 72792, 72793, 72794, 72795, 72796, 72797, 72798, 72799 were selected from the ORFs having the nucleotide sequence represented by SEQ ID NO: 6008.

The probes having the nucleotide sequence represented by SEQ ID NOS: 72800, 72801, 72802, 72803, 72804, 72805, 72806, 72807, 72808, 72809, 72810 were selected from the ORFs having the nucleotide sequence represented by SEQ ID NO: 6009.

The probes having the nucleotide sequence represented by SEQ ID NOS: 72811, 72812, 72813, 72814, 72815, 72816, 72817, 72818, 72819, 72820, 72821 were selected from the ORFs having the nucleotide sequence represented by SEQ ID NO: 6010.

The probes having the nucleotide sequence represented by SEQ ID NOS: 72822, 72823, 72824, 72825, 72826, 72827, 72828, 72829, 72830, 72831, 72832 were selected from the ORFs having the nucleotide sequence represented by SEQ ID NO: 6011.

The probes having the nucleotide sequence represented by SEQ ID NOS: 72833, 72834, 72835, 72836, 72837, 72838, 72839, 72840, 72841, 72842, 72843 were selected from the ORFs having the nucleotide sequence represented by SEQ ID NO: 6012.

The probes having the nucleotide sequence represented by SEQ ID NOS: 72844, 72845, 72846, 72847, 72848, 72849, 72850, 72851, 72852, 72853, 72854 were selected from the ORFs having the nucleotide sequence represented by SEQ ID NO: 6013.

The probes having the nucleotide sequence represented by SEQ ID NOS: 72855, 72856, 72857, 72858, 72859, 72860, 72861, 72862, 72863, 72864, 72865 were selected from the ORFs having the nucleotide-sequence represented by SEQ ID NO: 6014.

The probes having the nucleotide sequence represented by SEQ ID NOS: 72866, 72867, 72868, 72869, 72870, 72871, 72872, 72873, 72874, 72875, 72876, 72877, 72878, 72879, 72880, 72881, 72882, 72883, 72884, 72885, 72886, 72887 were selected from the ORFs having the nucleotide sequence represented by SEQ ID NO: 6015.

The probes having the nucleotide sequence represented by SEQ ID NOS: 72888, 72889, 72890, 72891, 72892, 72893, 72894, 72895, 72896, 72897, 72898 were selected from the ORFs having the nucleotide sequence represented by SEQ ID NO: 6016.

The probes having the nucleotide sequence represented by SEQ ID NOS: 72899, 72900, 72901, 72902, 72903, 72904, 72905, 72906, 72907, 72908, 72909 were selected from the ORFs having the nucleotide sequence represented by SEQ ID NO: 6017.

The probes having the nucleotide sequence represented by SEQ ID NOS: 72910, 72911, 72912, 72913, 72914, 72915, 72916, 72917, 72918, 72919, 72920 were selected from the ORFs having the nucleotide sequence represented by SEQ ID NO: 6018.

The probes having the nucleotide sequence represented by SEQ ID NOS: 72921, 72922, 72923, 72924, 72925, 72926, 72927, 72928, 72929, 72930, 72931 were selected from the ORFs having the nucleotide sequence represented by SEQ ID NO: 6019.

The probes having the nucleotide sequence represented by SEQ ID NOS: 72932, 72933, 72934, 72935, 72936, 72937, 72938, 72939, 72940, 72941, 72942 were selected from the ORFs having the nucleotide sequence represented by SEQ ID NO: 6020.

The probes having the nucleotide sequence represented by SEQ ID NOS: 72943, 72944, 72945, 72946, 72947, 72948, 72949, 72950, 72951, 72952, 72953 were selected from the ORFs having the nucleotide sequence represented by SEQ ID NO: 6021.

The probes having the nucleotide sequence represented by SEQ ID NOS: 72954, 72955, 72956, 72957, 72958, 72959, 72960, 72961, 72962, 72963, 72964 were selected from the ORFs having the nucleotide sequence represented by SEQ ID NO: 6022.

The probes having the nucleotide sequence represented by SEQ ID NOS: 72965, 72966, 72967, 72968, 72969, 72970, 72971, 72972, 72973, 72974, 72975 were selected from the ORFs having the nucleotide sequence represented by SEQ ID NO: 6023.

The probes having the nucleotide sequence represented by SEQ ID NOS: 72976, 72977, 72978, 72979, 72980, 72981, 72982, 72983, 72984, 72985, 72986 were selected from the ORFs having the nucleotide sequence represented by SEQ ID NO: 6024.

The probes having the nucleotide sequence represented by SEQ ID NOS: 72987, 72988, 72989, 72990, 72991, 72992, 72993, 72994, 72995, 72996, 72997 were selected from the ORFs having the nucleotide sequence represented by SEQ ID NO: 6025.

The probes having the nucleotide sequence represented by SEQ ID NOS: 72998, 72999, 73000, 73001, 73002, 73003, 73004, 73005, 73006, 73007, 73008 were selected from the ORFs having the nucleotide sequence represented by SEQ ID NO: 6026.

The probes having the nucleotide sequence represented by SEQ ID NOS: 73009, 73010, 73011, 73012, 73013, 73014, 73015, 73016, 73017, 73018, 73019 were selected from the ORFs having the nucleotide sequence represented by SEQ ID NO: 6027.

The probes having the nucleotide sequence represented by SEQ ID NOS: 73020, 73021, 73022, 73023, 73024, 73025, 73026, 73027, 73028, 73029, 73030 were selected from the ORFs having the nucleotide sequence represented by SEQ ID NO: 6028.

The probes having the nucleotide sequence represented by SEQ ID NOS: 73031, 73032, 73033, 73034, 73035, 73036, 73037, 73038, 73039, 73040, 73041 were selected from the ORFs having the nucleotide sequence represented by SEQ ID NO: 6029.

The probes having the nucleotide sequence represented by SEQ ID NOS: 73042, 73043, 73044, 73045, 73046, 73047, 73048, 73049, 73050, 73051, 73052 were selected from the ORFs having the nucleotide sequence represented by SEQ ID NO: 6030.

The probes having the nucleotide sequence represented by SEQ ID NOS: 73053, 73054, 73055, 73056, 73057, 73058, 73059, 73060, 73061, 73062, 73063 were selected from the ORFs having the nucleotide sequence represented by SEQ ID NO: 6031.

The probes having the nucleotide sequence represented by SEQ ID NOS: 73064, 73065, 73066, 73067, 73068, 73069, 73070, 73071, 73072, 73073, 73074 were selected from the ORFs having the nucleotide sequence represented by SEQ ID NO: 6032.

The probes having the nucleotide sequence represented by SEQ ID NOS: 73075, 73076, 73077, 73078, 73079, 73080, 73081, 73082 were selected from the ORFs having the nucleotide sequence represented by SEQ ID NO: 6033.

The probes having the nucleotide sequence represented by SEQ ID NOS: 73083, 73084, 73085, 73086, 73087, 73088, 73089, 73090, 73091, 73092, 73093 were selected from the ORFs having the nucleotide sequence represented by SEQ ID NO: 6034.

The probes having the nucleotide sequence represented by SEQ ID NOS: 73094, 73095, 73096, 73097, 73098, 73099, 73100, 73101, 73102, 73103, 73104 were selected from the ORFs having the nucleotide sequence represented by SEQ ID NO: 6035.

The probes having the nucleotide sequence represented by SEQ ID NOS: 73105, 73106, 73107, 73108, 73109, 73110, 73111, 73112, 73113, 73114, 73115 were selected from the ORFs having the nucleotide sequence represented by SEQ ID NO: 6036.

The probes having the nucleotide sequence represented by SEQ ID NOS: 73116, 73117, 73118, 73119, 73120, 73121, 73122, 73123, 73124, 73125, 73126 were selected from the ORFs having the nucleotide sequence represented by SEQ ID NO: 6037.

The probes having the nucleotide sequence represented by SEQ ID NOS: 73127, 73128, 73129, 73130, 73131, 73132, 73133, 73134, 73135, 73136, 73137 were selected from the ORFs having the nucleotide sequence represented by SEQ ID NO: 6038.

The probes having the nucleotide sequence represented by SEQ ID NOS: 73138, 73139, 73140, 73141, 73142, 73143, 73144, 73145, 73146, 73147, 73148 were selected from the ORFs having the nucleotide sequence represented by SEQ ID NO: 6039.

The probes having the nucleotide sequence represented by SEQ ID NOS: 73149, 73150, 73151, 73152, 73153, 73154, 73155, 73156, 73157, 73158, 73159 were selected from the ORFs having the nucleotide sequence represented by SEQ ID NO: 6040.

The probes having the nucleotide sequence represented by SEQ ID NOS: 73160, 73161, 73162, 73163, 73164, 73165, 73166, 73167, 73168, 73169, 73170 were selected from the ORFs having the nucleotide sequence represented by SEQ ID NO: 6041.

The probes having the nucleotide sequence represented by SEQ ID NOS: 73171, 73172, 73173, 73174, 73175, 73176, 73177, 73178, 73179, 73180, 73181 were selected from the ORFs having the nucleotide sequence represented by SEQ ID NO: 6042.

The probes having the nucleotide sequence represented by SEQ ID NOS: 73182, 73183, 73184, 73185, 73186, 73187, 73188, 73189, 73190, 73191, 73192 were selected from the ORFs having the nucleotide sequence represented by SEQ ID NO: 6043.

The probes having the nucleotide sequence represented by SEQ ID NOS: 73193, 73194, 73195, 73196, 73197, 73198, 73199, 73200, 73201, 73202, 73203 were selected from the ORFs having the nucleotide sequence represented by SEQ ID NO: 6044.

The probes having the nucleotide sequence represented by SEQ ID NOS: 73204, 73205, 73206, 73207, 73208, 73209, 73210, 73211, 73212, 73213, 73214 were selected from the ORFs having the nucleotide sequence represented by SEQ ID NO: 6045.

The probes having the nucleotide sequence represented by SEQ ID NOS: 73215, 73216, 73217, 73218, 73219, 73220, 73221, 73222, 73223, 73224, 73225 were selected from the ORFs having the nucleotide sequence represented by SEQ ID NO: 6046.

The probes having the nucleotide sequence represented by SEQ ID NOS: 73226, 73227, 73228, 73229, 73230, 73231, 73232, 73233, 73234, 73235, 73236 were selected from the ORFs having the nucleotide sequence represented by SEQ ID NO: 6047.

The probes having the nucleotide sequence represented by SEQ ID NOS: 73237, 73238, 73239, 73240, 73241, 73242, 73243, 73244, 73245, 73246, 73247 were selected from the ORFs having the nucleotide sequence represented by SEQ ID NO: 6048.

The probes having the nucleotide sequence represented by SEQ ID NOS: 73248, 73249, 73250, 73251, 73252, 73253, 73254, 73255, 73256, 73257, 73258 were selected from the ORFs having the nucleotide sequence represented by SEQ ID NO: 6049.

The probes having the nucleotide sequence represented by SEQ ID NOS: 73259, 73260, 73261, 73262, 73263, 73264, 73265, 73266, 73267, 73268, 73269 were selected from the ORFs having the nucleotide sequence represented by SEQ ID NO: 6050.

The probes having the nucleotide sequence represented by SEQ ID NOS: 73270, 73271, 73272, 73273, 73274, 73275, 73276, 73277, 73278, 73279, 73280 were selected from the ORFs having the nucleotide sequence represented by SEQ ID NO: 6051.

The probes having the nucleotide sequence represented by SEQ ID NOS: 73281, 73282, 73283, 73284, 73285, 73286, 73287, 73288, 73289, 73290, 73291 were selected from the ORFs having the nucleotide sequence represented by SEQ ID NO: 6052.

The probes having the nucleotide sequence represented by SEQ ID NOS: 73292, 73293, 73294, 73295, 73296, 73297, 73298, 73299, 73300, 73301, 73302 were selected from the ORFs having the nucleotide sequence represented by SEQ ID NO: 6053.

The probes having the nucleotide sequence represented by SEQ ID NOS: 73303, 73304, 73305, 73306, 73307, 73308, 73309, 73310, 73311, 73312, 73313 were selected from the ORFs having the nucleotide sequence represented by SEQ ID NO: 6054.

The probes having the nucleotide sequence represented by SEQ ID NOS: 73314, 73315, 73316, 73317, 73318, 73319, 73320, 73321, 73322, 73323, 73324 were selected from the ORFs having the nucleotide sequence represented by SEQ ID NO: 6055.

The probes having the nucleotide sequence represented by SEQ ID NOS: 73325, 73326, 73327, 73328, 73329, 73330, 73331, 73332, 73333, 73334, 73335 were selected from the ORFs having the nucleotide sequence represented by SEQ ID NO: 6056.

The probes having the nucleotide sequence represented by SEQ ID NOS: 73336, 73337, 73338, 73339, 73340, 73341, 73342, 73343, 73344, 73345, 73346 were selected from the ORFs having the nucleotide sequence represented by SEQ ID NO: 6057.

The probes having the nucleotide sequence represented by SEQ ID NOS: 73347, 73348, 73349, 73350, 73351, 73352, 73353, 73354, 73355, 73356, 73357 were selected from the ORFs having the nucleotide sequence represented by SEQ ID NO: 6058.

The probes having the nucleotide sequence represented by SEQ ID NOS: 73358, 73359, 73360, 73361, 73362, 73363, 73364, 73365, 73366, 73367, 73368 were selected from the ORFs having the nucleotide sequence represented by SEQ ID NO: 6059.

The probes having the nucleotide sequence represented by SEQ ID NOS: 73369, 73370, 73371, 73372, 73373, 73374, 73375, 73376, 73377, 73378, 73379 were selected from the ORFs having the nucleotide sequence represented by SEQ ID NO: 6060.

The probes having the nucleotide sequence represented by SEQ ID NOS: 73380, 73381, 73382, 73383, 73384, 73385, 73386, 73387, 73388, 73389, 73390 were selected from the ORFs having the nucleotide sequence represented by SEQ ID NO: 6061.

The probes having the nucleotide sequence represented by SEQ ID NOS: 73391, 73392, 73393, 73394, 73395, 73396, 73397, 73398, 73399, 73400, 73401 were selected from the ORFs having the nucleotide sequence represented by SEQ ID NO: 6062.

The probes having the nucleotide sequence represented by SEQ ID NOS: 73402, 73403, 73404, 73405, 73406, 73407, 73408, 73409, 73410, 73411, 73412 were selected from the ORFs having the nucleotide sequence represented by SEQ ID NO: 6063.

The probes having the nucleotide sequence represented by SEQ ID NOS: 73413, 73414, 73415, 73416, 73417, 73418, 73419, 73420, 73421, 73422, 73423 were selected from the ORFs having the nucleotide sequence represented by SEQ ID NO: 6064.

The probes having the nucleotide sequence represented by SEQ ID NOS: 73424, 73425, 73426, 73427, 73428, 73429, 73430, 73431, 73432, 73433, 73434 were selected from the ORFs having the nucleotide sequence represented by SEQ ID NO: 6065.

The probes having the nucleotide sequence represented by SEQ ID NOS: 73435, 73436, 73437, 73438, 73439, 73440, 73441, 73442, 73443, 73444, 73445 were selected from the ORFs having the nucleotide sequence represented by SEQ ID NO: 6066.

The probes having the nucleotide sequence represented by SEQ ID NOS: 73446, 73447, 73448, 73449, 73450, 73451, 73452, 73453, 73454, 73455, 73456 were selected from the ORFs having the nucleotide sequence represented by SEQ ID NO: 6067.

The probes having the nucleotide sequence represented by SEQ ID NOS: 73457, 73458, 73459, 73460, 73461, 73462, 73463, 73464, 73465, 73466, 73467 were selected from the ORFs having the nucleotide sequence represented by SEQ ID NO: 6068.

The probes having the nucleotide sequence represented by SEQ ID NOS: 73468, 73469, 73470, 73471, 73472, 73473, 73474, 73475, 73476, 73477, 73478 were selected from the ORFs having the nucleotide sequence represented by SEQ ID NO: 6069.

The probes having the nucleotide sequence represented by SEQ ID NOS: 73479, 73480, 73481, 73482, 73483, 73484, 73485, 73486, 73487, 73488, 73489 were selected from the ORFs having the nucleotide sequence represented by SEQ ID NO: 6070.

The probes having the nucleotide sequence represented by SEQ ID NOS: 73490, 73491, 73492, 73493, 73494, 73495, 73496, 73497, 73498, 73499, 73500 were selected from the ORFs having the nucleotide sequence represented by SEQ ID NO: 6071.

The probes having the nucleotide sequence represented by SEQ ID NOS: 73501, 73502, 73503, 73504, 73505, 73506, 73507, 73508, 73509, 73510, 73511 were selected from the ORFs having the nucleotide sequence represented by SEQ ID NO: 6072.

The probes having the nucleotide sequence represented by SEQ ID NOS: 73512, 73513, 73514, 73515, 73516, 73517, 73518, 73519, 73520, 73521, 73522 were selected from the ORFs having the nucleotide sequence represented by SEQ ID NO: 6073.

The probes having the nucleotide sequence represented by SEQ ID NOS: 73523, 73524, 73525, 73526, 73527, 73528, 73529, 73530, 73531, 73532, 73533 were selected from the ORFs having the nucleotide sequence represented by SEQ ID NO: 6074.

The probes having the nucleotide sequence represented by SEQ ID NOS: 73534, 73535, 73536, 73537, 73538, 73539, 73540, 73541, 73542, 73543, 73544 were selected from the ORFs having the nucleotide sequence represented by SEQ ID NO: 6075.

The probes having the nucleotide sequence represented by SEQ ID NOS: 73545, 73546, 73547, 73548, 73549, 73550, 73551, 73552, 73553, 73554, 73555 were selected from the ORFs having the nucleotide sequence represented by SEQ ID NO: 6076.

The probes having the nucleotide sequence represented by SEQ ID NOS: 73556, 73557, 73558, 73559, 73560, 73561, 73562, 73563, 73564, 73565, 73566 were selected from the ORFs having the nucleotide sequence represented by SEQ ID NO: 6077.

The probes having the nucleotide sequence represented by SEQ ID NOS: 73567, 73568, 73569, 73570, 73571, 73572, 73573, 73574, 73575, 73576, 73577 were selected from the ORFs having the nucleotide sequence represented by SEQ ID NO: 6078.

The probes having the nucleotide sequence represented by SEQ ID NOS: 73578, 73579, 73580, 73581, 73582, 73583, 73584, 73585, 73586, 73587, 73588 were selected from the ORFs having the nucleotide sequence represented by SEQ ID NO: 6079.

The probes having the nucleotide sequence represented by SEQ ID NOS: 73589, 73590, 73591, 73592, 73593, 73594, 73595, 73596, 73597, 73598, 73599 were selected from the ORFs having the nucleotide sequence represented by SEQ ID NO: 6080.

The probes having the nucleotide sequence represented by SEQ ID NOS: 73600, 73601, 73602, 73603, 73604, 73605, 73606, 73607, 73608, 73609, 73610 were selected from the ORFs having the nucleotide sequence represented by SEQ ID NO: 6081.

The probes having the nucleotide sequence represented by SEQ ID NOS: 73611, 73612, 73613, 73614, 73615, 73616, 73617, 73618, 73619, 73620, 73621 were selected from the ORFs having the nucleotide sequence represented by SEQ ID NO: 6082.

The probes having the nucleotide sequence represented by SEQ ID NOS: 73622, 73623, 73624, 73625, 73626, 73627, 73628, 73629, 73630, 73631, 73632 were selected from the ORFs having the nucleotide sequence represented by SEQ ID NO: 6083.

The probes having the nucleotide sequence represented by SEQ ID NOS: 73633, 73634, 73635, 73636, 73637, 73638, 73639, 73640, 73641, 73642, 73643 were selected from the ORFs having the nucleotide sequence represented by SEQ ID NO: 6084.

The probes having the nucleotide sequence represented by SEQ ID NOS: 73644, 73645, 73646, 73647, 73648, 73649, 73650, 73651, 73652, 73653, 73654 were selected from the ORFs having the nucleotide sequence represented by SEQ ID NO: 6085.

The probes having the nucleotide sequence represented by SEQ ID NOS: 73655, 73656, 73657, 73658, 73659, 73660, 73661, 73662, 73663, 73664, 73665 were selected from the ORFs having the nucleotide sequence represented by SEQ ID NO: 6086.

The probes having the nucleotide sequence represented by SEQ ID NOS: 73666, 73667, 73668, 73669, 73670, 73671, 73672, 73673, 73674, 73675, 73676 were selected from the ORFs having the nucleotide sequence represented by SEQ ID NO: 6087.

The probes having the nucleotide sequence represented by SEQ ID NOS: 73677, 73678, 73679, 73680, 73681, 73682, 73683, 73684, 73685, 73686, 73687 were selected from the ORFs having the nucleotide sequence represented by SEQ ID NO: 6088.

The probes having the nucleotide sequence represented by SEQ ID NOS: 73688, 73689, 73690, 73691, 73692, 73693, 73694, 73695, 73696, 73697, 73698 were selected from the ORFs having the nucleotide sequence represented by SEQ ID NO: 6089.

The probes having the nucleotide sequence represented by SEQ ID NOS: 73699, 73700, 73701, 73702, 73703, 73704, 73705, 73706, 73707, 73708, 73709 were selected from the ORFs having the nucleotide sequence represented by SEQ ID NO: 6090.

The probes having the nucleotide sequence represented by SEQ ID NOS: 73710, 73711, 73712, 73713, 73714, 73715, 73716, 73717, 73718, 73719, 73720 were selected from the ORFs having the nucleotide sequence represented by SEQ ID NO: 6091.

The probes having the nucleotide sequence represented by SEQ ID NOS: 73721, 73722, 73723, 73724, 73725, 73726, 73727, 73728, 73729, 73730, 73731 were selected from the ORFs having the nucleotide sequence represented by SEQ ID NO: 6092.

The probes having the nucleotide sequence represented by SEQ ID NOS: 73732, 73733, 73734, 73735, 73736, 73737, 73738, 73739, 73740, 73741, 73742 were selected from the ORFs having the nucleotide sequence represented by SEQ ID NO: 6093.

The probes having the nucleotide sequence represented by SEQ ID NOS: 73743, 73744, 73745, 73746, 73747, 73748, 73749, 73750, 73751, 73752, 73753 were selected from the ORFs having the nucleotide sequence represented by SEQ ID NO: 6094.

The probes having the nucleotide sequence represented by SEQ ID NOS: 73754, 73755, 73756, 73757, 73758, 73759, 73760, 737616 73762, 73763, 73764 were selected from the ORFs having the nucleotide sequence represented by SEQ ID NO: 6095.

The probes having the nucleotide sequence represented by SEQ ID NOS: 73765, 73766, 73767, 73768, 73769, 73770, 73771, 73772, 73773, 73774, 73775 were selected from the ORFs having the nucleotide sequence represented by SEQ ID NO: 6096.

The probes having the nucleotide sequence represented by SEQ ID NOS: 73776, 73777, 73778, 73779, 73780, 73781, 73782, 73783, 73784, 73785, 73786 were selected from the ORFs having the nucleotide sequence represented by SEQ ID NO: 6097.

The probes having the nucleotide sequence represented by SEQ ID NOS: 73787, 73788, 73789, 73790, 73791, 73792, 73793, 73794, 73795, 73796, 73797 were selected from the ORFs having the nucleotide sequence represented by SEQ ID NO: 6098.

The probes having the nucleotide sequence represented by SEQ ID NOS: 73798, 73799, 73800, 73801, 73802, 73803, 73804, 73805, 73806, 73807, 73808 were selected from the ORFs having the nucleotide sequence represented by SEQ ID NO: 6099.

The probes having the nucleotide sequence represented by SEQ ID NOS: 73809, 73810, 73811, 73812, 73813, 73814, 73815, 73816, 73817, 73818, 73819 were selected from the ORFs having the nucleotide sequence represented by SEQ ID NO: 6100.

The probes having the nucleotide sequence represented by SEQ ID NOS: 73820, 73821, 73822, 73823, 73824, 73825, 73826, 73827, 73828, 73829, 73830 were selected from the ORFs having the nucleotide sequence represented by SEQ ID NO: 6101.

The probes having the nucleotide sequence represented by SEQ ID NOS: 73831, 73832, 73833, 73834, 73835, 73836, 73837, 73838, 73839, 73840, 73841 were selected from the ORFs having the nucleotide sequence represented by SEQ ID NO: 6102.

The probes having the nucleotide sequence represented by SEQ ID NOS: 73842, 73843, 73844, 73845, 73846, 73847, 73848, 73849, 73850, 73851, 73852 were selected from the ORFs having the nucleotide sequence represented by SEQ ID NO: 6103.

The probes having the nucleotide sequence represented by SEQ ID NOS: 73853, 73854, 73855, 73856, 73857, 73858, 73859, 73860, 73861, 73862, 73863 were selected from the ORFs having the nucleotide sequence represented by SEQ ID NO: 6104.

The probes having the nucleotide sequence represented by SEQ ID NOS: 73864, 73865, 73866, 73867, 73868, 73869, 73870, 73871, 73872, 73873, 73874 were selected from the ORFs having the nucleotide sequence represented by SEQ ID NO: 6105.

The probes having the nucleotide sequence represented by SEQ ID NOS: 73875, 73876, 73877, 73878, 73879, 73880, 73881, 73882, 73883, 73884, 73885 were selected from the ORFs having the nucleotide sequence represented by SEQ ID NO: 6106.

The probes having the nucleotide sequence represented by SEQ ID NOS: 73886, 73887, 73888, 73889, 73890, 73891, 73892, 73893, 73894, 73895, 73896 were selected from the ORFs having the nucleotide sequence represented by SEQ ID NO: 6107.

The probes having the nucleotide sequence represented by SEQ ID NOS: 73897, 73898, 73899, 73900, 73901, 73902, 73903, 73904, 73905, 73906, 73907 were selected from the ORFs having the nucleotide sequence represented by SEQ ID NO: 6108.

The probes having the nucleotide sequence represented by SEQ ID NOS: 73908, 73909, 73910, 73911, 73912, 73913, 73914, 73915, 73916, 73917, 73918 were selected from the ORFs having the nucleotide sequence represented by SEQ ID NO: 6109.

The probes having the nucleotide sequence represented by SEQ ID NOS: 73919, 73920, 73921, 73922, 73923, 73924, 73925, 73926, 73927, 73928, 73929 were selected from the ORFs having the nucleotide sequence represented by SEQ ID NO: 6110.

The probes having the nucleotide sequence represented by SEQ ID NOS: 73930, 73931, 73932, 73933, 73934, 73935, 73936, 73937, 73938, 73939, 73940 were selected from the ORFs having the nucleotide sequence represented by SEQ ID NO: 6111.

The probes having the nucleotide sequence represented by SEQ ID NOS: 73941, 73942, 73943, 73944, 73945, 73946, 73947, 73948, 73949, 73950, 73951 were selected from the ORFs having the nucleotide sequence represented by SEQ ID NO: 6112.

The probes having the nucleotide sequence represented by SEQ ID NOS: 73952, 73953, 73954, 73955, 73956, 73957, 73958, 73959, 73960, 73961, 73962 were selected from the ORFs having the nucleotide sequence represented by SEQ ID NO: 6113.

The probes having the nucleotide sequence represented by SEQ ID NOS: 73963, 73964, 73965, 73966, 73967, 73968, 73969, 73970, 73971, 73972, 73973 were selected from the ORFs having the nucleotide sequence represented by SEQ ID NO: 6114.

The probes having the nucleotide sequence represented by SEQ ID NOS: 73974, 73975, 73976, 73977, 73978, 73979, 73980, 73981, 73982, 73983, 73984 were selected from the ORFs having the nucleotide sequence represented by SEQ ID NO: 6115.

The probes having the nucleotide sequence represented by SEQ ID NOS: 73985, 73986, 73987, 73988, 73989, 73990, 73991, 73992, 73993, 73994, 73995 were selected from the ORFs having the nucleotide sequence represented by SEQ ID NO: 6116.

The probes having the nucleotide sequence represented by SEQ ID NOS: 73996, 73997, 73998, 73999, 74000, 74001, 74002, 74003, 74004, 74005, 74006 were selected from the ORFs having the nucleotide sequence represented by SEQ ID NO: 6117.

The probes having the nucleotide sequence represented by SEQ ID NOS: 74007, 74008, 74009, 74010, 74011, 74012, 74013, 74014, 74015, 74016, 74017 were selected from the ORFs having the nucleotide sequence represented by SEQ ID NO: 6118.

The probes having the nucleotide sequence represented by SEQ ID NOS: 74018, 74019, 74020, 74021, 74022, 74023, 74024, 74025, 74026, 74027, 74028 were selected from the ORFs having the nucleotide sequence represented by SEQ ID NO: 6119.

The probes having the nucleotide sequence represented by SEQ ID NOS: 74029, 74030, 74031, 74032, 74033, 74034, 74035, 74036, 74037, 74038, 74039 were selected from the ORFs having the nucleotide sequence represented by SEQ ID NO: 6120.

The probes having the nucleotide sequence represented by SEQ ID NOS: 74040, 74041, 74042, 74043, 74044, 74045, 74046, 74047, 74048, 74049, 74050 were selected from the ORFs having the nucleotide sequence represented by SEQ ID NO: 6121.

The probes having the nucleotide sequence represented by SEQ ID NOS: 74051, 74052, 74053, 74054, 74055, 74056, 74057, 74058, 74059, 74060, 74061 were selected from the ORFs having the nucleotide sequence represented by SEQ ID NO: 6122.

The probes having the nucleotide sequence represented by SEQ ID NOS: 74062, 74063, 74064, 74065, 74066, 74067, 74068, 74069, 74070, 74071, 74072 were selected from the ORFs having the nucleotide sequence represented by SEQ ID NO: 6123.

The probes having the nucleotide sequence represented by SEQ ID NOS: 74073, 74074, 74075, 74076, 74077, 74078, 74079, 74080, 74081, 74082, 74083 were selected from the ORFs having the nucleotide sequence represented by SEQ ID NO: 6124.

The probes having the nucleotide sequence represented by SEQ ID NOS: 74084, 74085, 74086, 74087, 74088, 74089,

74090, 74091, 74092, 74093, 74094 were selected from the ORFs having the nucleotide sequence represented by SEQ ID NO: 6125.

The probes having the nucleotide sequence represented by SEQ ID NOS: 74095, 74096, 74097, 74098, 74099, 74100, 74101, 74102, 74103, 74104, 74105 were selected from the ORFs having the nucleotide sequence represented by SEQ ID NO: 6126.

The probes having the nucleotide sequence represented by SEQ ID NOS: 74106, 74107, 74108, 74109, 74110, 74111, 74112, 74113, 74114, 74115, 74116 were selected from the ORFs having the nucleotide sequence represented by SEQ ID NO: 6127.

The probes having the nucleotide sequence represented by SEQ ID NOS: 74117, 74118, 74119, 74120, 74121, 74122, 74123, 74124, 74125, 74126, 74127 were selected from the ORFs having the nucleotide sequence represented by SEQ ID NO: 6128.

The probes having the nucleotide sequence represented by SEQ ID NOS: 74128, 74129, 74130, 74131, 74132, 74133, 74134, 74135, 74136, 74137, 74138 were selected from the ORFs having the nucleotide sequence represented by SEQ ID NO: 6129.

The probes having the nucleotide sequence represented by SEQ ID NOS: 74139, 74140, 74141, 74142, 74143, 74144, 74145, 74146, 74147, 74148, 74149 were selected from the ORFs having the nucleotide sequence represented by SEQ ID NO: 6130.

The probes having the nucleotide sequence represented by SEQ ID NOS: 74150, 74151, 74152, 74153, 74154, 74155, 74156, 74157, 74158, 74159, 74160 were selected from the ORFs having the nucleotide sequence represented by SEQ ID NO: 6131.

The probes having the nucleotide sequence represented by SEQ ID NOS: 74161, 74162, 74163, 74164, 74165, 74166, 74167, 74168, 74169, 74170, 74171 were selected from the ORFs having the nucleotide sequence represented by SEQ ID NO: 6132.

The probes having the nucleotide sequence represented by SEQ ID NOS: 74172, 74173, 74174, 74175, 74176, 74177, 74178, 74179, 74180, 74181, 74182 were selected from the ORFs having the nucleotide sequence represented by SEQ ID NO: 6133.

The probes having the nucleotide sequence represented by SEQ ID NOS: 74183, 74184, 74185, 74186, 74187, 74188, 74189, 74190, 74191, 74192, 74193 were selected from the ORFs having the nucleotide sequence represented by SEQ ID NO: 6134.

The probes having the nucleotide sequence represented by SEQ ID NOS: 74194, 74195, 74196, 74197, 74198, 74199, 74200, 74201, 74202, 74203, 74204 were selected from the ORFs having the nucleotide sequence represented by SEQ ID NO: 6135.

The probes having the nucleotide sequence represented by SEQ ID NOS: 74205, 74206, 74207, 74208, 74209, 74210, 74211, 74212, 74213, 74214, 74215 were selected from the ORFs having the nucleotide sequence represented by SEQ ID NO: 6136.

The probes having the nucleotide sequence represented by SEQ ID NOS: 74216, 74217, 74218, 74219, 74220, 74221, 74222, 74223, 74224, 74225, 74226 were selected from the ORFs having the nucleotide sequence represented by SEQ ID NO: 6137.

The probes having the nucleotide sequence represented by SEQ ID NOS: 74227, 74228, 74229, 74230, 74231, 74232, 74233, 74234, 74235, 74236, 74237 were selected from the ORFs having the nucleotide sequence represented by SEQ ID NO: 6138.

The probes having the nucleotide sequence represented by SEQ ID NOS: 74238, 74239, 74240, 74241, 74242, 74243, 74244, 74245, 74246, 74247, 74248 were selected from the ORFs having the nucleotide sequence represented by SEQ ID NO: 6139.

The probes having the nucleotide sequence represented by SEQ ID NOS: 74249, 74250, 74251, 74252, 74253, 74254, 74255, 74256, 74257, 74258, 74259 were selected from the ORFs having the nucleotide sequence represented by SEQ ID NO: 6140.

The probes having the nucleotide sequence represented by SEQ ID NOS: 74260, 74261, 74262, 74263, 74264, 74265, 74266, 74267, 74268, 74269, 74270 were selected from the ORFs having the nucleotide sequence represented by SEQ ID NO: 6141.

The probes having the nucleotide sequence represented by SEQ ID NOS: 74271, 74272, 74273, 74274, 74275, 74276, 74277, 74278, 74279, 74280, 74281 were selected from the ORFs having the nucleotide sequence represented by SEQ ID NO: 6142.

the probes having the nucleotide sequence represented by SEQ ID NOS: 74282, 74283, 74284, 74285, 74286, 74287, 74288, 74289, 74290, 74291, 74292 were selected from the ORFs having the nucleotide sequence represented by SEQ ID NO: 6143.

The probes having the nucleotide sequence represented by SEQ ID NOS: 74293, 74294, 74295, 74296, 74297, 74298, 74299, 74300, 74301, 74302, 74303 were selected from the ORFs having the nucleotide sequence represented by SEQ ID NO: 6144.

The probes having the nucleotide sequence represented by SEQ ID NOS: 74304, 74305, 74306, 74307, 74308, 74309, 74310, 74311, 74312, 74313, 74314 were selected from the ORFs having the nucleotide sequence represented by SEQ ID NO: 6145.

The probes having the nucleotide sequence represented by SEQ ID NOS: 74315, 74316, 74317, 74318, 74319, 74320, 74321, 74322, 74323, 74324, 74325 were selected from the ORFs having the nucleotide sequence represented by SEQ ID NO: 6146.

The probes having the nucleotide sequence represented by SEQ ID NOS: 74326, 74327, 74328, 74329, 74330, 74331, 74332, 74333, 74334, 74335, 74336 were selected from the ORFs having the nucleotide sequence represented by SEQ ID NO: 6147.

The probes having the nucleotide sequence represented by SEQ ID NOS: 74337, 74338, 74339, 74340, 74341, 74342, 74343, 74344, 74345, 74346, 74347 were selected from the ORFs having the nucleotide sequence represented by SEQ ID NO: 6148.

The probes having the nucleotide sequence represented by SEQ ID NOS: 74348, 74349, 74350, 74351, 74352, 74353, 74354, 74355, 74356, 74357, 74358 were selected from the ORFs having the nucleotide sequence represented by SEQ ID NO: 6149.

The probes having the nucleotide sequence represented by SEQ ID NOS: 74359, 74360, 74361, 74362, 74363, 74364, 74365, 74366, 74367, 74368, 74369 were selected from the ORFs having the nucleotide sequence represented by SEQ ID NO: 6150.

The probes having the nucleotide sequence represented by SEQ ID NOS: 74370, 74371, 74372, 74373, 74374, 74375, 74376, 74377, 74378, 74379, 74380 were selected from the ORFs having the nucleotide sequence represented by SEQ ID NO: 6151.

The probes having the nucleotide sequence represented by SEQ ID NOS: 74381, 74382, 74383, 74384, 74385, 74386, 74387, 74388, 74389, 74390, 74391 were selected from the ORFs having the nucleotide sequence represented by SEQ ID NO: 6152.

The probes having the nucleotide sequence represented by SEQ ID NOS: 74392, 74393, 74394, 74395, 74396, 74397, 74398, 74399, 74400, 74401, 74402 were selected from the ORFs having the nucleotide sequence represented by SEQ ID NO: 6153.

The probes having the nucleotide sequence represented by SEQ ID NOS: 74403, 74404, 74405, 74406, 74407, 74408, 74409, 74410, 74411, 74412, 74413 were selected from the ORFs having the nucleotide sequence represented by SEQ ID NO: 6154.

The probes having the nucleotide sequence represented by SEQ ID NOS: 74414, 74415, 74416, 74417, 74418, 74419, 74420, 74421, 74422, 74423, 74424 were selected from the ORFs having the nucleotide sequence represented by SEQ ID NO: 6155.

The probes having the nucleotide sequence represented by SEQ ID NOS: 74425, 74426, 74427, 74428, 74429, 74430, 74431, 74432, 74433, 74434, 74435 were selected from the ORFs having the nucleotide sequence represented by SEQ ID NO: 6156.

The probes having the nucleotide sequence represented by SEQ ID NOS: 74436, 74437, 74438, 74439, 74440, 74441, 74442, 74443, 74444, 74445, 74446 were selected from the ORFs having the nucleotide sequence represented by SEQ ID NO: 6157.

The probes having the nucleotide sequence represented by SEQ ID NOS: 74447, 74448, 74449, 74450, 74451, 74452, 74453, 74454, 74455, 74456, 74457 were selected from the ORFs having the nucleotide sequence represented by SEQ ID NO: 6158.

The probes having the nucleotide sequence represented by SEQ ID NOS: 74458, 74459, 74460, 74461, 74462, 74463, 74464, 74465, 74466, 74467, 74468 were selected from the ORFs having the nucleotide sequence represented by SEQ ID NO: 6159.

The probes having the nucleotide sequence represented by SEQ ID NOS: 74469, 74470, 74471, 74472, 74473, 74474, 74475, 74476, 74477, 74478, 74479 were selected from the ORFs having the nucleotide sequence represented by SEQ ID NO: 6160.

The probes having the nucleotide sequence represented by SEQ ID NOS: 74480, 74481, 74482, 74483, 74484, 74485, 74486, 74487, 74488, 74489, 74490 were selected from the ORFs having the nucleotide sequence represented by SEQ ID NO: 6161.

The probes having the nucleotide sequence represented by SEQ ID NOS: 74491, 74492, 74493, 74494, 74495, 74496, 74497, 74498, 74499, 74500, 74501 were selected from the ORFs having the nucleotide sequence represented by SEQ ID NO: 6162.

The probes having the nucleotide sequence represented by SEQ ID NOS: 74502, 74503, 74504, 74505, 74506, 74507, 74508, 74509, 74510, 74511, 74512 were selected from the ORFs having the nucleotide sequence represented by SEQ ID NO: 6163.

The probes having the nucleotide sequence represented by SEQ ID NOS: 74513, 74514, 74515, 74516, 74517, 74518, 74519, 74520, 74521, 74522, 74523 were selected from the ORFs having the nucleotide sequence represented by SEQ ID NO: 6164.

The probes having the nucleotide sequence represented by SEQ ID NOS: 74524, 74525, 74526, 74527, 74528, 74529, 74530, 74531, 74532, 74533, 74534 were selected from the ORFs having the nucleotide sequence represented by SEQ ID NO: 6165.

The probes having the nucleotide sequence represented by SEQ ID NOS: 74535, 74536, 74537, 74538, 74539, 74540, 74541, 74542, 74543, 74544, 74545 were selected from the ORFs having the nucleotide sequence represented by SEQ ID NO: 6166.

The probes having the nucleotide sequence,represented by SEQ ID NOS: 74546, 74547, 74548, 74549, 74550, 74551, 74552, 74553, 74554, 74555, 74556 were selected from the ORFs having the nucleotide sequence represented by SEQ ID NO: 6167.

The probes having the nucleotide sequence represented by SEQ ID NOS: 74557, 74558, 74559, 74560, 74561, 74562, 74563, 74564, 74565, 74566, 74567 were selected from the ORFs having the nucleotide sequence represented by SEQ ID NO: 6168.

The probes having the nucleotide sequence represented by SEQ ID NOS: 74568, 74569, 74570, 74571, 74572, 74573, 74574, 74575, 74576, 74577, 74578 were selected from the ORFs having the nucleotide sequence represented by SEQ ID NO: 6169.

The probes having the nucleotide sequence represented by SEQ ID NOS: 74579, 74580, 74581, 74582, 74583, 74584, 74585, 74586, 74587, 74588, 74589 were selected from the ORFs having the nucleotide sequence represented by SEQ ID NO: 6170.

The probes having the nucleotide sequence represented by SEQ ID NOS: 74590, 74591, 74592, 74593, 74594, 74595, 74596, 74597, 74598, 74599, 74600 were selected from the ORFs having the nucleotide sequence represented by SEQ ID NO: 6171.

The probes having the nucleotide sequence represented by SEQ ID NOS: 74601, 74602, 74603, 74604, 74605, 74606, 74607, 74608, 74609, 74610, 74611 were selected from the ORFs having the nucleotide sequence represented by SEQ ID NO: 6172.

The probes having the nucleotide sequence represented by SEQ ID NOS: 74612, 74613, 74614, 74615, 74616, 74617, 74618, 74619, 74620, 74621, 74622 were selected from the ORFs having the nucleotide sequence represented by SEQ ID NO: 6173.

The probes having the nucleotide sequence represented by SEQ ID NOS: 74623, 74624, 74625, 74626, 74627, 74628, 74629, 74630, 74631, 74632, 74633 were selected from the ORFs having the nucleotide sequence represented by SEQ ID NO: 6174.

The probes having the nucleotide sequence represented by SEQ ID NOS: 74634, 74635, 74636, 74637, 74638, 74639, 74640, 74641, 74642, 74643, 74644 were selected from the ORFs having the nucleotide sequence represented by SEQ ID NO: 6175.

The probes having the nucleotide sequence represented by SEQ ID NOS: 74645, 74646, 74647, 74648, 74649, 74650, 74651, 74652, 74653, 74654, 74655 were selected from the ORFs having the nucleotide sequence represented by SEQ ID NO: 6176.

The probes having the nucleotide sequence represented by SEQ ID NOS: 74656, 74657, 74658, 74659, 74660, 74661, 74662, 74663, 74664, 74665, 74666, 74667, 74668, 74669, 74670, 74671, 74672, 74673, 74674, 74675, 74676, 74677 were selected from the ORFs having the nucleotide sequence represented by SEQ ID NO: 6177.

The probes having the nucleotide sequence represented by SEQ ID NOS: 74678, 74679, 74680, 74681, 74682, 74683, 74684, 74685, 74686, 74687, 74688 were selected from the ORFs having the nucleotide sequence represented by SEQ ID NO: 6178.

The probes having the nucleotide sequence represented by SEQ ID NOS: 74689, 74690, 74691, 74692, 74693, 74694, 74695, 74696, 74697, 74698, 74699 were selected from the ORFs having the nucleotide sequence represented by SEQ ID NO: 6179.

The probes having the nucleotide sequence represented by SEQ ID NOS: 74700, 74701, 74702, 74703, 74704, 74705, 74706, 74707, 74708, 74709, 74710 were selected from the ORFs having the nucleotide sequence represented by SEQ ID NO: 6180.

The probes having the nucleotide sequence represented by SEQ ID NOS: 74711, 74712, 74713, 74714, 74715, 74716, 74717, 74718, 74719, 74720, 74721 were selected from the ORFs having the nucleotide sequence represented by SEQ ID NO: 6181.

The probes having the nucleotide sequence represented by SEQ ID NOS: 74722, 74723, 74724, 74725, 74726, 74727, 74728, 74729, 74730, 74731, 74732 were selected from the ORFs having the nucleotide sequence represented by SEQ ID NO: 6182.

The probes having the nucleotide sequence represented by SEQ ID NOS: 74733, 74734, 74735, 74736, 74737, 74738, 74739, 74740, 74741, 74742, 74743 were selected from the ORFs having the nucleotide sequence represented by SEQ ID NO: 6183.

The probes having the nucleotide sequence represented by SEQ ID NOS: 74744, 74745, 74746, 74747, 74748, 74749, 74750, 74751, 74752, 74753, 74754 were selected from the ORFs having the nucleotide sequence represented by SEQ ID NO: 6184.

The probes having the nucleotide sequence represented by SEQ ID NOS: 74755, 74756, 74757, 74758, 74759, 74760, 74761, 74762, 74763, 74764, 74765 were selected from the ORFs having the nucleotide sequence represented by SEQ ID NO: 6185.

The probes having the nucleotide sequence represented by SEQ ID NOS: 74766, 74767, 74768, 74769, 74770, 74771, 74772, 74773, 74774, 74775, 74776 were selected from the ORFs having the nucleotide sequence represented by SEQ ID NO: 6186.

The probes having the nucleotide sequence represented by SEQ ID NOS: 74777, 74778, 74779, 74780, 74781, 74782, 74783, 74784, 74785, 74786, 74787 were selected from the ORFs having the nucleotide sequence represented by SEQ ID NO: 6187.

The probes having the nucleotide sequence represented by SEQ ID NOS: 74788, 74789, 74790, 74791, 74792, 74793, 74794, 74795, 74796, 74797, 74798 were selected from the ORFs having the nucleotide sequence represented by SEQ ID NO: 6188.

The probes having the nucleotide sequence represented by SEQ ID NOS: 74799, 74800, 74801, 74802, 74803, 74804, 74805, 74806, 74807, 74808, 74809 were selected from the ORFs having the nucleotide sequence represented by SEQ ID NO: 6189.

The probes having the nucleotide sequence represented by SEQ ID NOS: 74810, 74811, 74812, 74813, 74814, 74815, 74816, 74817, 74818, 74819, 74820 were selected from the ORFs having the nucleotide sequence represented by SEQ ID NO: 6190.

The probes having the nucleotide sequence represented by SEQ ID NOS: 74821, 74822, 74823, 74824, 74825, 74826, 74827, 74828, 74829, 74830, 74831 were selected from the ORFs having the nucleotide sequence represented by SEQ ID NO: 6191.

The probes having the nucleotide sequence represented by SEQ ID NOS: 74832, 74833, 74834, 74835, 74836, 74837, 74838, 74839, 74840, 74841, 74842 were selected from the ORFs having the nucleotide sequence represented by SEQ ID NO: 6192.

The probes having the nucleotide sequence represented by SEQ ID NOS: 74843, 74844, 74845, 74846, 74847, 74848, 74849, 74850, 74851, 74852, 74853 were selected from the ORFs having the nucleotide sequence represented by SEQ ID NO: 6193.

The probes having the nucleotide sequence represented by SEQ ID NOS: 74854, 74855, 74856, 74857, 74858, 74859, 74860, 74861, 74862, 74863, 74864, 74865, 74866, 74867, 74868, 74869, 74870, 74871, 74872, 74873, 74874, 74875 were selected from the ORFs having the nucleotide sequence represented by SEQ ID NO: 6194.

The probes having the nucleotide sequence represented by SEQ ID NOS: 74876, 74877, 74878, 74879, 74880, 74881, 74882, 74883, 74884, 74885, 74886 were selected from the ORFs having the nucleotide sequence represented by SEQ ID NO: 6195.

The probes having the nucleotide sequence represented by SEQ ID NOS: 74887, 74888, 74889, 74890, 74891, 74892, 74893, 74894, 74895, 74896, 74897 were selected from the ORFs having the nucleotide sequence represented by SEQ ID NO: 6196.

The probes having the nucleotide sequence represented by SEQ ID NOS: 74898, 74899, 74900, 74901, 74902, 74903, 74904, 74905, 74906, 74907, 74908 were selected from the ORFs having the nucleotide sequence represented by SEQ ID NO: 6197.

The probes having the nucleotide sequence represented by SEQ ID NOS: 74909, 74910, 74911, 74912, 74913, 74914, 74915, 74916, 74917, 74918, 74919 were selected from the ORFs having the nucleotide sequence represented by SEQ ID NO: 6198.

The probes having the nucleotide sequence represented by SEQ ID NOS: 74920, 74921, 74922, 74923, 74924, 74925, 74926, 74927, 74928, 74929, 74930 were selected from the ORFs having the nucleotide sequence represented by SEQ ID NO: 6199.

The probes having the nucleotide sequence represented by SEQ ID NOS: 74931, 74932, 74933, 74934, 74935, 74936, 74937, 74938, 74939, 74940, 74941 were selected from the ORFs having the nucleotide sequence represented by SEQ ID NO: 6200.

The probes having the nucleotide sequence represented by SEQ ID NOS: 74942, 74943, 74944, 74945, 74946, 74947, 74948, 74949, 74950, 74951, 74952 were selected from the ORFs having the nucleotide sequence represented by SEQ ID NO: 6201.

The probes having the nucleotide sequence represented by SEQ ID NOS: 74953, 74954, 74955, 74956, 74957, 74958, 74959, 74960, 74961, 74962, 74963 were selected from the ORFs having the nucleotide sequence represented by SEQ ID NO: 6202.

The probes having the nucleotide sequence represented by SEQ ID NOS: 74964, 74965, 74966, 74967, 74968, 74969, 74970, 74971, 74972, 74973, 74974 were selected from the ORFs having the nucleotide sequence represented by SEQ ID NO: 6203.

The probes having the nucleotide sequence represented by SEQ ID NOS: 74975, 74976, 74977, 74978, 74979, 74980, 74981, 74982, 74983, 74984, 74985 were selected from the ORFs having the nucleotide sequence represented by SEQ ID NO: 6204.

The probes having the nucleotide sequence represented by SEQ ID NOS: 74986, 74987, 74988, 74989, 74990, 74991, 74992, 74993, 74994, 74995, 74996 were selected from the ORFs having the nucleotide sequence represented by SEQ ID NO: 6205.

The probes having the nucleotide sequence represented by SEQ ID NOS: 74997, 74998, 74999, 75000, 75001, 75002, 75003, 75004, 75005, 75006, 75007 were selected from the ORFs having the nucleotide sequence represented by SEQ ID NO: 6206.

The probes having the nucleotide sequence represented by SEQ ID NOS: 75008, 75009, 75010, 75011, 75012, 75013, 75014, 75015, 75016, 75017, 75018 were selected from the ORFs having the nucleotide sequence represented by SEQ ID NO: 6207.

The probes having the nucleotide sequence represented by SEQ ID NOS: 75019, 75020, 75021, 75022, 75023, 75024, 75025, 75026, 75027, 75028, 75029 were selected from the ORFs having the nucleotide sequence represented by SEQ ID NO: 6208.

The probes having the nucleotide sequence represented by SEQ ID NOS: 75030, 75031, 75032, 75033, 75034, 75035, 75036, 75037, 75038, 75039, 75040 were selected from the ORFs having the nucleotide sequence represented by SEQ ID NO: 6209.

The probes having the nucleotide sequence represented by SEQ ID NOS: 75041, 75042, 75043, 75044, 75045, 75046, 75047, 75048, 75049, 75050, 75051 were selected from the ORFs having the nucleotide sequence represented by SEQ ID NO: 6210.

The probes having the nucleotide sequence represented by SEQ ID NOS: 75052, 75053, 75054, 75055, 75056, 75057, 75058, 75059, 75060, 75061, 75062 were selected from the ORFs having the nucleotide sequence represented by SEQ ID NO: 6211.

The probes having the nucleotide sequence represented by SEQ ID NOS: 75063, 75064, 75065, 75066, 75067, 75068, 75069, 75070, 75071, 75072, 75073 were selected from the ORFs having the nucleotide sequence represented by SEQ ID NO: 6212.

The probes having the nucleotide sequence represented by SEQ ID NOS: 75074, 75075, 75076, 75077, 75078, 75079, 75080, 75081, 75082, 75083, 75084 were selected from the ORFs having the nucleotide sequence represented by SEQ ID NO: 6213.

The probes having the nucleotide sequence represented by SEQ ID NOS: 75085, 75086, 75087, 75088, 75089, 75090, 75091, 75092, 75093, 75094, 75095 were selected from the ORFs having the nucleotide sequence represented by SEQ ID NO: 6214.

The probes having the nucleotide sequence represented by SEQ ID NOS: 75096, 75097, 75098, 75099, 75100, 75101, 75102, 75103, 75104, 75105, 75106 were selected from the ORFs having the nucleotide sequence represented by SEQ ID NO: 6215.

The probes having the nucleotide sequence represented by SEQ ID NOS: 75107, 75108, 75109, 75110, 75111, 75112, 75113, 75114, 75115, 75116, 75117 were selected from the ORFs having the nucleotide sequence represented by SEQ ID NO: 6216.

The probes having the nucleotide sequence represented by SEQ ID NOS: 75118, 75119, 75120, 75121, 75122, 75123, 75124, 75125, 75126, 75127, 75128 were selected from the ORFs having the nucleotide sequence represented by SEQ ID NO: 6217.

The probes having the nucleotide sequence represented by SEQ ID NOS: 75129, 75130, 75131, 75132, 75133, 75134, 75135, 75136, 75137, 75138, 75139 were selected from the ORFs having the nucleotide sequence represented by SEQ ID NO: 6218.

The probes having the nucleotide sequence represented by SEQ ID NOS: 75140, 75141, 75142, 75143, 75144, 75145, 75146, 75147, 75148, 75149, 75150 were selected from the ORFs having the nucleotide sequence represented by SEQ ID NO: 6219.

The probes having the nucleotide sequence represented by SEQ ID NOS: 75151, 75152, 75153, 75154, 75155, 75156, 75157, 75158, 75159, 75160 were selected from the ORFs having the nucleotide sequence represented by SEQ ID NO: 6220.

The probes having the nucleotide sequence represented by SEQ ID NOS: 75161, 75162, 75163, 75164, 75165, 75166, 75167, 75168, 75169, 75170, 75171 were selected from the ORFs having the nucleotide sequence represented by SEQ ID NO: 6221.

The probes having the nucleotide sequence represented by SEQ ID NOS: 75172, 75173, 75174, 75175, 75176, 75177, 75178, 75179, 75180, 75181, 75182 were selected from the ORFs having the nucleotide sequence represented by SEQ ID NO: 6222.

The probes having the nucleotide sequence represented by SEQ ID NOS: 75183, 75184, 75185, 75186, 75187, 75188, 75189, 75190, 75191, 75192, 75193 were selected from the ORFs having the nucleotide sequence represented by SEQ ID NO: 6223.

The probes having the nucleotide sequence represented by SEQ ID NOS: 75194, 75195, 75196, 75197, 75198, 75199, 75200, 75201, 75202, 75203, 75204 were selected from the ORFs having the nucleotide sequence represented by SEQ ID NO: 6224.

The probes having the nucleotide sequence represented by SEQ ID NOS: 75205, 75206, 75207, 75208, 75209, 75210, 75211, 75212, 75213, 75214, 75215 were selected from the ORFs having the nucleotide sequence represented by SEQ ID NO: 6225.

The probes having the nucleotide sequence represented by SEQ ID NOS: 75216, 75217, 75218, 75219, 75220, 75221, 75222, 75223, 75224, 75225, 75226 were selected from the ORFs having the nucleotide sequence represented by SEQ ID NO: 6226.

The probes having the nucleotide sequence represented by SEQ ID NOS: 75227, 75228, 75229, 75230, 75231, 75232, 75233, 75234, 75235, 75236, 75237 were selected from the ORFs having the nucleotide sequence represented by SEQ ID NO: 6227.

The probes having the nucleotide sequence represented by SEQ ID NOS: 75238, 75239, 75240, 75241, 75242, 75243, 75244, 75245, 75246, 75247, 75248 were selected from the ORFs having the nucleotide sequence represented by SEQ ID NO: 6228.

The probes having the nucleotide sequence represented by SEQ ID NOS: 75249, 75250, 75251, 75252, 75253, 75254, 75255, 75256, 75257, 75258, 75259 were selected from the ORFs having the nucleotide sequence represented by SEQ ID NO: 6229.

The probes having the nucleotide sequence represented by SEQ ID NOS: 75260, 75261, 75262, 75263, 75264, 75265, 75266, 75267, 75268, 75269, 75270 were selected from the ORFs having the nucleotide sequence represented by SEQ ID NO: 6230.

The probes having the nucleotide sequence represented by SEQ ID NOS: 75271, 75272, 75273, 75274, 75275, 75276, 75277, 75278, 75279, 75280, 75281 were selected from the ORFs having the nucleotide sequence represented by SEQ ID NO: 6231.

The probes having the nucleotide sequence represented by SEQ ID NOS: 75282, 75283, 75284, 75285, 75286, 75287, 75288, 75289, 75290, 75291, 75292 were selected from the ORFs having the nucleotide sequence represented by SEQ ID NO: 6232.

The probes having the nucleotide sequence represented by SEQ ID NOS: 75293, 75294, 75295, 75296, 75297, 75298, 75299, 75300, 75301, 75302, 75303 were selected from the ORFs having the nucleotide sequence represented by SEQ ID NO: 6233.

The probes having the nucleotide sequence represented by SEQ ID NOS: 75304, 75305, 75306, 75307, 75308, 75309, 75310, 75311, 75312, 75313, 75314 were selected from the ORFs having the nucleotide sequence represented by SEQ ID NO: 6234.

The probes having the nucleotide sequence represented by SEQ ID NOS: 75315, 75316, 75317, 75318, 75319, 75320,

75321, 75322, 75323, 75324, 75325 were selected from the ORFs having the nucleotide sequence represented by SEQ ID NO: 6235.

The probes having the nucleotide sequence represented by SEQ ID NOS: 75326, 75327, 75328, 75329, 75330, 75331,

75332, 75333, 75334, 75335, 75336 were selected from the ORFs having the nucleotide sequence represented by SEQ ID NO: 6236.

The probes from 7448 DNA sequences of non-Sc type ORFs described as above (1-B) were as follows:

The probes having the nucleotide sequence represented by SEQ ID NOS: 82785, 82786, 82787, 82788, 82789, 82790, 82791, 82792, 82793, 82794, 82795 were selected from the ORFs having the nucleotide sequence represented by SEQ ID NO: 75337.

The probes having the nucleotide sequence represented by SEQ ID NOS: 82796, 82797, 82798, 82799, 82800, 82801, 82802, 82803, 82804, 82805, 82806 were selected from the ORFs having the nucleotide sequence represented by SEQ ID NO: 75338.

The probes having the nucleotide sequence represented by SEQ ID NOS: 82807, 82808, 82809, 82810, 82811, 82812, 82813, 82814, 82815, 82816, 82817 were selected from the ORFs having the nucleotide sequence represented by SEQ ID NO: 75339.

The probes having the nucleotide sequence represented by SEQ ID NOS: 82818, 82819, 82820, 82821, 82822, 82823, 82824, 82825, 82826, 82827, 82828 were selected from the ORFs having the nucleotide sequence represented by SEQ ID NO: 75340.

The probes having the nucleotide sequence represented by SEQ ID NOS: 82829, 82830, 82831, 82832, 82833, 82834, 82835, 82836, 82837, 82838, 82839 were selected from the ORFs having the nucleotide sequence represented by SEQ ID NO: 75341.

The probes having the nucleotide sequence represented by SEQ ID NOS: 82840, 82841, 82842, 82843, 82844, 82845, 82846, 82847, 82848, 82849, 82850 were selected from the ORFs having the nucleotide sequence represented by SEQ ID NO: 75342.

The probes having the nucleotide sequence represented by SEQ ID NOS: 82851, 82852, 82853, 82854, 82855, 82856, 82857, 82858, 82859, 82860, 82861 were selected from the ORFs having the nucleotide sequence represented by SEQ ID NO: 75343.

The probes having the nucleotide sequence represented by SEQ ID NOS: 82862, 82863, 82864, 82865, 82866, 82867, 82868, 82869, 82870, 82871, 82872 were selected from the ORFs having the nucleotide sequence represented by SEQ ID NO: 75344.

The probes having the nucleotide sequence represented by SEQ ID NOS: 82873, 82874, 82875, 82876, 82877, 82878, 82879, 82880, 82881, 82882, 82883 were selected from the ORFs having the nucleotide sequence represented by SEQ ID NO: 75345.

The probes having the nucleotide sequence represented by SEQ ID NOS: 82884, 82885, 82886, 82887, 82888, 82889, 82890, 82891, 82892, 82893, 82894 were selected from the ORFs having the nucleotide sequence represented by SEQ ID NO: 75346.

The probes having the nucleotide sequence represented by SEQ ID NOS: 82895, 82896, 82897, 82898, 82899, 82900, 82901, 82902, 82903, 82904, 82905 were selected from the ORFs having the nucleotide sequence represented by SEQ ID NO: 75347.

The probes having the nucleotide sequence represented by SEQ ID NOS: 82906, 82907, 82908, 82909, 82910, 82911, 82912, 82913, 82914, 82915, 82916 were selected from the ORFs having the nucleotide sequence represented by SEQ ID NO: 75348.

The probes having the nucleotide sequence represented by SEQ ID NOS: 82917, 82918, 82919, 82920, 82921, 82922, 82923, 82924, 82925, 82926, 82927 were selected from the ORFs having the nucleotide sequence represented by SEQ ID NO: 75349.

The probes having the nucleotide sequence represented by SEQ ID NOS: 82928, 82929, 82930, 82931, 82932, 82933, 82934, 82935, 82936, 82937, 82938 were selected from the ORFs having the nucleotide sequence represented by SEQ ID NO: 75350.

The probes having the nucleotide sequence represented by SEQ ID NOS: 82939, 82940, 82941, 82942, 82943, 82944, 82945, 82946, 82947, 82948, 82949 were selected from the ORFs having the nucleotide sequence represented by SEQ ID NO: 75351.

The probes having the nucleotide sequence represented by SEQ ID NOS: 82950, 82951, 82952, 82953, 82954, 82955, 82956, 82957, 82958, 82959, 82960 were selected from the ORFs having the nucleotide sequence represented by SEQ ID NO:. 75352.

The probes having the nucleotide sequence represented by SEQ ID NOS: 82961, 82962, 82963, 82964, 82965, 82966, 82967, 82968, 82969, 82970, 82971 were selected from the ORFs having the nucleotide sequence represented by SEQ ID NO: 75353.

The probes having the nucleotide sequence represented by SEQ ID NOS: 82972, 82973, 82974, 82975, 82976, 82977, 82978, 82979, 82980, 82981, 82982 were selected from the ORFs having the nucleotide sequence represented by SEQ ID NO: 75354.

The probes having the nucleotide sequence represented by SEQ ID NOS: 82983, 82984, 82985, 82986, 82987, 82988, 82989, 82990, 82991, 82992, 82993 were selected from the ORFs having the nucleotide sequence represented by SEQ ID NO: 75355.

The probes having the nucleotide sequence represented by SEQ ID NOS: 82994, 82995, 82996, 82997, 82998, 82999, 83000, 83001, 83002, 83003, 83004 were selected from the ORFs having the nucleotide sequence represented by SEQ ID NO: 75356.

The probes having the nucleotide sequence represented by SEQ ID NOS: 83005, 83006, 83007, 83008, 83009, 83010, 83011, 83012, 83013, 83014, 83015 were selected from the ORFs having the nucleotide sequence represented by SEQ ID NO: 75357.

The probes having the nucleotide sequence represented by SEQ ID NOS: 83016, 83017, 83018, 83019, 83020, 83021, 83022, 83023, 83024, 83025, 83026 were selected from the ORFs having the nucleotide sequence represented by SEQ ID NO: 75358.

The probes having the nucleotide sequence represented by SEQ ID NOS: 83027, 83028, 83029, 83030, 83031, 83032, 83033, 83034, 83035, 83036, 83037 were selected from the ORFs having the nucleotide sequence represented by SEQ ID NO: 75359.

The probes having the nucleotide sequence represented by SEQ ID NOS: 83038, 83039, 83040, 83041, 83042, 83043, 83044, 83045, 83046, 83047, 83048 were selected from the ORFs having the nucleotide sequence represented by SEQ ID NO: 75360.

The probes having the nucleotide sequence represented by SEQ ID NOS: 83049, 83050, 83051, 83052, 83053, 83054, 83055, 83056, 83057, 83058, 83059 were selected from the ORFs having the nucleotide sequence represented by SEQ ID NO: 75361.

The probes having the nucleotide sequence represented by SEQ ID NOS: 83060, 83061, 83062, 83063, 83064, 83065, 83066, 83067, 83068, 83069, 83070 were selected from the ORFs having the nucleotide sequence represented by SEQ ID NO: 75362.

The probes having the nucleotide sequence represented by SEQ ID NOS: 83071, 83072, 83073, 83074, 83075, 83076, 83077, 83078, 83079, 83080, 83081 were selected from the ORFs having the nucleotide sequence represented by SEQ ID NO: 75363.

The probes having the nucleotide sequence represented by SEQ ID NOS: 83082, 83083, 83084, 83085, 83086, 83087, 83088, 83089, 83090, 83091, 83092 were selected from the ORFs having the nucleotide sequence represented by SEQ ID NO: 75364.

The probes having the nucleotide sequence represented by SEQ ID NOS: 83093, 83094, 83095, 83096, 83097, 83098, 83099, 83100, 83101, 83102, 83103 were selected from the ORFs having the nucleotide sequence represented by SEQ ID NO: 75365.

The probes having the nucleotide sequence represented by SEQ ID NOS: 83104, 83105, 83106, 83107, 83108, 83109, 83110, 83111, 83112, 83113, 83114 were selected from the ORFs having the nucleotide sequence represented by SEQ ID NO: 75366.

The probes having the nucleotide sequence represented by SEQ ID NOS: 83115, 83116, 83117, 83118, 83119, 83120, 83121, 83122, 83123, 83124, 83125 were selected from the ORFs having the nucleotide sequence represented by SEQ ID NO: 75367.

The probes having the nucleotide sequence represented by SEQ ID NOS: 83126, 83127, 83128, 83129, 83130, 83131, 83132, 83133, 83134, 83135, 83136 were selected from the ORFs having the nucleotide sequence represented by SEQ ID NO: 75368.

The probes having the nucleotide sequence represented by SEQ ID NOS: 83137, 83138, 83139, 83140, 83141, 83142, 83143, 83144, 83145, 83146, 83147 were selected from the ORFs having the nucleotide sequence represented by SEQ ID NO: 75369.

The probes having the nucleotide sequence represented by SEQ ID NOS: 83148, 83149, 83150, 83151, 83152, 83153, 83154, 83155, 83156, 83157, 83158 were selected from the ORFs having the nucleotide sequence represented by SEQ ID NO: 75370.

The probes having the nucleotide sequence represented by SEQ ID NOS: 83159, 83160, 83161, 83162, 83163, 83164, 83165, 83166, 83167, 83168, 83169 were selected from the ORFs having the nucleotide sequence represented by SEQ ID NO: 75371.

The probes having the nucleotide sequence represented by SEQ ID NOS: 83170, 83171, 83172, 83173, 83174, 83175, 83176, 83177, 83178, 83179, 83180, 83181, 83182, 83183, 83184, 83185, 83186, 83187, 83188, 83189, 83190, 83191 were selected from the ORFs having the nucleotide sequence represented by SEQ ID NO: 75372.

The probes having the nucleotide sequence represented by SEQ ID NOS: 83192, 83193, 83194, 83195, 83196, 83197, 83198, 83199, 83200, 83201, 83202 were selected from the ORFs having the nucleotide sequence represented by SEQ ID NO: 75373.

The probes having the nucleotide sequence represented by SEQ ID NOS: 83203, 83204, 83205, 83206, 83207, 83208, 83209, 83210, 83211, 83212, 83213 were selected from the ORFs having the nucleotide sequence represented by SEQ ID NO: 75374.

The probes having the nucleotide sequence represented by SEQ ID NOS: 83214, 83215, 83216, 83217, 83218, 83219, 83220, 83221, 83222, 83223, 83224 were selected from the ORFs having the nucleotide sequence represented by SEQ ID NO: 75375.

The probes having the nucleotide sequence represented by SEQ ID NOS: 83225, 83226, 83227, 83228, 83229, 83230, 83231, 83232, 83233, 83234, 83235 were selected from the ORFs having the nucleotide sequence represented by SEQ ID NO: 75376.

The probes having the nucleotide sequence represented by SEQ ID NOS: 83236, 83237, 83238, 83239, 83240, 83241, 83242, 83243, 83244, 83245, 83246 were selected from the ORFs having the nucleotide sequence represented by SEQ ID NO: 75377.

The probes having the nucleotide sequence represented by SEQ ID NOS: 83247, 83248, 83249, 83250, 83251, 83252, 83253, 83254, 83255, 83256, 83257 were selected from the ORFs having the nucleotide sequence represented by SEQ ID NO: 75378.

The probes having the nucleotide sequence represented by SEQ ID NOS: 83258, 83259, 83260, 83261, 83262, 83263, 83264, 83265, 83266, 83267, 83268 were selected from the ORFs having the nucleotide sequence represented by SEQ ID NO: 75379.

The probes having the nucleotide sequence represented by SEQ ID NOS: 83269, 83270, 83271, 83272, 83273, 83274, 83275, 83276, 83277, 83278, 83279 were selected from the ORFs having the nucleotide sequence represented by SEQ ID NO: 75380.

The probes having the nucleotide sequence-represented by SEQ ID NOS: 83280, 83281, 83282, 83283, 83284, 83285, 83286, 83287, 83288, 83289, 83290 were selected from the ORFs having the nucleotide sequence represented by SEQ ID NO: 75381.

The probes having the nucleotide sequence represented by SEQ ID NOS: 83291, 83292, 83293, 83294, 83295, 83296, 83297, 83298, 83299, 83300, 83301 were selected from the ORFs having the nucleotide sequence represented by SEQ ID NO: 75382.

The probes having the nucleotide sequence represented by SEQ ID NOS: 83302, 83303, 83304, 83305, 83306, 83307, 83308, 83309, 83310, 83311, 83312 were selected from the ORFs having the nucleotide sequence represented by SEQ ID NO: 75383.

The probes having the nucleotide sequence represented by SEQ ID NOS: 83313, 83314, 83315, 83316, 83317, 83318, 83319, 83320, 83321, 83322, 83323 were selected from the ORFs having the nucleotide sequence represented by SEQ ID NO: 75384.

The probes having the nucleotide sequence represented by SEQ ID NOS: 83324, 83325, 83326, 83327, 83328, 83329, 83330, 83331, 83332, 83333, 83334 were selected from the ORFs having the nucleotide sequence represented by SEQ ID NO: 75385.

The probes having the nucleotide sequence represented by SEQ ID NOS: 83335, 83336, 83337, 83338, 83339, 83340, 83341, 83342, 83343, 83344, 83345 were selected from the ORFs having the nucleotide sequence represented by SEQ ID NO: 75386.

The probes having the nucleotide sequence represented by SEQ ID NOS: 83346, 83347, 83348, 83349, 83350, 83351, 83352, 83353, 83354, 83355, 83356 were selected from the ORFs having the nucleotide sequence represented by SEQ ID NO: 75387.

The probes having the nucleotide sequence represented by SEQ ID NOS: 83357, 83358, 83359, 83360, 83361, 83362, 83363, 83364, 83365, 83366, 83367 were selected from the ORFs having the nucleotide sequence represented by SEQ ID NO: 75388.

The probes having the nucleotide sequence represented by SEQ ID NOS: 83368, 83369, 83370, 83371, 83372, 83373, 83374, 83375, 83376, 83377, 83378 were selected from the ORFs having the nucleotide sequence represented by SEQ ID NO: 75389.

The probes having the nucleotide sequence represented by SEQ ID NOS: 83379, 83380, 83381, 83382, 83383, 83384, 83385, 83386, 83387, 83388, 83389 were selected from the ORFs having the nucleotide sequence represented by SEQ ID NO: 75390.

The probes having the nucleotide sequence represented by SEQ ID NOS: 83390, 83391, 83392, 83393, 83394, 83395, 83396, 83397, 83398, 83399 were selected from the ORFs having the nucleotide sequence represented by SEQ ID NO: 75391.

The probes having the nucleotide sequence represented by SEQ ID NOS: 83400, 83401, 83402, 83403, 83404, 83405, 83406, 83407, 83408, 83409, 83410 were selected from the ORFs having the nucleotide sequence represented by SEQ ID NO: 75392.

The probes having the nucleotide sequence represented by SEQ ID NOS: 83411, 83412, 83413, 83414, 83415, 83416, 83417, 83418, 83419, 83420, 83421 were selected from the ORFs having the nucleotide sequence represented by SEQ ID NO: 75393.

The probes having the nucleotide sequence represented by SEQ ID NOS: 83422, 83423, 83424, 83425, 83426, 83427, 83428, 83429, 83430, 83431, 83432 were selected from the ORFs having the nucleotide sequence represented by SEQ ID NO: 75394.

The probes having the nucleotide sequence represented by SEQ ID NOS: 83433, 83434, 83435, 83436, 83437, 83438, 83439, 83440, 83441, 83442, 83443 were selected from the ORFs having the nucleotide sequence represented by SEQ ID NO: 75395.

The probes having the nucleotide sequence represented by SEQ ID NOS: 83444, 83445, 83446, 83447, 83448, 83449, 83450, 83451, 83452, 83453, 83454 were selected from the ORFs having the nucleotide sequence represented by SEQ ID NO: 75396.

The probes having the nucleotide sequence represented by SEQ ID NOS: 83455, 83456, 83457, 83458, 83459, 83460, 83461, 83462, 83463, 83464, 83465 were selected from the ORFs having the nucleotide sequence represented by SEQ ID NO: 75397.

The probes having the nucleotide sequence represented by SEQ ID NOS: 83466, 83467, 83468, 83469, 83470, 83471, 83472, 83473, 83474, 83475, 83476 were selected from the ORFs having the nucleotide sequence represented by SEQ ID NO: 75398.

The probes having the nucleotide sequence represented by SEQ ID NOS: 83477, 83478, 83479, 83480, 83481, 83482, 83483, 83484, 83485, 83486, 83487 were selected from the ORFs having the nucleotide sequence represented by SEQ ID NO: 75399.

The probes having the nucleotide sequence represented by SEQ ID NOS: 83488, 83489, 83490, 83491, 83492, 83493, 83494, 83495, 83496, 83497, 83498 were selected from the ORFs having the nucleotide sequence represented by SEQ ID NO: 75400.

The probes having the nucleotide sequence represented by SEQ ID NOS: 83499, 83500, 83501, 83502, 83503, 83504, 83505, 83506, 83507, 83508, 83509 were selected from the ORFs having the nucleotide sequence represented by SEQ ID NO: 75401.

The probes having the nucleotide sequence represented by SEQ ID NOS: 83510, 83511, 83512, 83513, 83514, 83515, 83516, 83517, 83518, 83519, 83520 were selected from the ORFs having the nucleotide sequence represented by SEQ ID NO: 75402.

The probes having the nucleotide sequence represented by SEQ ID NOS: 83521, 83522, 83523, 83524, 83525, 83526, 83527, 83528, 83529, 83530, 83531 were selected from the ORFs having the nucleotide sequence represented by SEQ ID NO: 75403.

The probes having the nucleotide sequence represented by SEQ ID NOS: 83532, 83533, 83534, 83535, 83536, 83537, 83538, 83539, 83540, 83541, 83542 were selected from the ORFs having the nucleotide sequence represented by SEQ ID NO: 75404.

The probes having the nucleotide sequence represented by SEQ ID NOS: 83543, 83544, 83545, 83546, 83547, 83548, 83549, 83550, 83551, 83552, 83553 were selected from the ORFs having the nucleotide sequence represented by SEQ ID NO: 75405.

The probes having the nucleotide sequence represented by SEQ ID NOS: 83554, 83555, 83556, 83557, 83558, 83559, 83560, 83561, 83562, 83563, 83564 were selected from the ORFs having the nucleotide sequence represented by SEQ ID NO: 75406.

The probes having the nucleotide sequence represented by SEQ ID NOS: 83565, 83566, 83567, 83568, 83569, 83570, 83571, 83572, 83573, 83574, 83575 were selected from the ORFs having the nucleotide sequence represented by SEQ ID NO: 75407.

The probes having the nucleotide sequence represented by SEQ ID NOS: 83576, 83577, 83578, 83579, 83580, 83581, 83582, 83583, 83584, 83585, 83586 were selected from the ORFs having the nucleotide sequence represented by SEQ ID NO: 75408.

The probes having the nucleotide sequence represented by SEQ ID NOS: 83587, 83588, 83589, 83590, 83591, 83592, 83593, 83594, 83595, 83596, 83597 were selected from the ORFs having the nucleotide sequence represented by SEQ ID NO: 75409.

The probes having the nucleotide sequence represented by SEQ ID NOS: 83598, 83599, 83600, 83601, 83602, 83603, 83604, 83605, 83606, 83607, 83608 were selected from the ORFs having the nucleotide sequence represented by SEQ ID NO: 75410.

The probes having the nucleotide sequence represented by SEQ ID NOS: 83609, 83610, 83611, 83612, 83613, 83614, 83615, 83616, 83617, 83618, 83619 were selected from the ORFs having the nucleotide sequence represented by SEQ ID NO: 75411.

The probes having the nucleotide sequence represented by SEQ ID NOS: 83620, 83621, 83622, 83623, 83624, 83625, 83626, 83627, 83628, 83629, 83630 were selected from the ORFs having the nucleotide sequence represented by SEQ ID NO: 75412.

The probes having the nucleotide sequence represented by SEQ ID NOS: 83631, 83632, 83633, 83634, 83635, 83636, 83637, 83638, 83639, 83640, 83641 were selected from the ORFs having the nucleotide sequence represented by SEQ ID NO: 75413.

The probes having the nucleotide sequence represented by SEQ ID NOS: 83642, 83643, 83644, 83645, 83646, 83647, 83648, 83649, 83650, 83651, 83652 were selected from the ORFs having the nucleotide sequence represented by SEQ ID NO: 75414.

The probes having the nucleotide sequence represented by SEQ ID NOS: 83653, 83654, 83655, 83656, 83657, 83658, 83659, 83660, 83661, 83662, 83663 were selected from the ORFs having the nucleotide sequence represented by SEQ ID NO: 75415.

The probes having the nucleotide sequence represented by SEQ ID NOS: 83664, 83665, 83666, 83667, 83668, 83669, 83670, 83671, 83672, 83673, 83674 were selected from the ORFs having the nucleotide sequence represented by SEQ ID NO: 75416.

The probes having the nucleotide sequence represented by SEQ ID NOS: 83675, 83676, 83677, 83678, 83679, 83680, 83681, 83682, 83683, 83684, 83685 were selected from the ORFs having the nucleotide sequence represented by SEQ ID NO: 75417.

The probes having the nucleotide sequence represented by SEQ ID NOS: 83686, 83687, 83688, 83689, 83690, 83691, 83692, 83693, 83694, 83695, 83696 were selected from the ORFs having the nucleotide sequence represented by SEQ ID NO: 75418.

The probes having the nucleotide sequence represented by SEQ ID NOS: 83697, 83698, 83699, 83700, 83701, 83702, 83703, 83704, 83705, 83706, 83707 were selected from the ORFs having the nucleotide sequence represented by SEQ ID NO: 75419.

The probes having the nucleotide sequence represented by SEQ ID NOS: 83708, 83709, 83710, 83711, 83712, 83713, 83714, 83715, 83716, 83717, 83718 were selected from the ORFs having the nucleotide sequence represented by SEQ ID NO: 75420.

The probes having the nucleotide sequence represented by SEQ ID NOS: 83719, 83720, 83721, 83722, 83723, 83724, 83725, 83726, 83727, 83728, 83729 were selected from the ORFs having the nucleotide sequence represented by SEQ ID NO: 75421.

The probes having the nucleotide sequence represented by SEQ ID NOS: 83730, 83731, 83732, 83733, 83734, 83735, 83736, 83737, 83738, 83739, 83740 were selected from the ORFs having the nucleotide sequence represented by SEQ ID NO: 75422.

The probes having the nucleotide sequence represented by SEQ ID NOS: 83741, 83742, 83743, 83744, 83745, 83746, 83747, 83748, 83749, 83750, 83751 were selected from the ORFs having the nucleotide sequence represented by SEQ ID NO: 75423.

The probes having the nucleotide sequence represented by SEQ ID NOS: 83752, 83753, 83754, 83755, 83756, 83757, 83758, 83759, 83760, 83761, 83762 were selected from the ORFs having the nucleotide sequence represented by SEQ ID NO: 75424.

The probes having the nucleotide sequence represented by SEQ ID NOS: 83763, 83764, 83765, 83766, 83767, 83768, 83769, 83770, 83771, 83772, 83773 were selected from the ORFs having the nucleotide sequence represented by SEQ ID NO: 75425.

The probes having the nucleotide sequence represented by SEQ ID NOS: 83774, 83775, 83776, 83777, 83778, 83779, 83780, 83781, 83782, 83783, 83784 were selected from the ORFs having the nucleotide sequence represented by SEQ ID NO: 75426.

The probes having the nucleotide sequence represented by SEQ ID NOS: 83785, 83786, 83787, 83788, 83789, 83790, 83791, 83792, 83793, 83794, 83795 were selected from the ORFs having the nucleotide sequence represented by SEQ ID NO: 75427.

The probes having the nucleotide sequence represented by SEQ ID NOS: 83796, 83797, 83798, 83799, 83800, 83801, 83802, 83803, 83804, 83805, 83806 were selected from the ORFs having the nucleotide sequence represented by SEQ ID NO: 75428.

The probes having the nucleotide sequence represented by SEQ ID NOS: 83807, 83808, 83809, 83810, 83811, 83812, 83813, 83814, 83815, 83816, 83817 were selected from the ORFs having the nucleotide sequence represented by SEQ ID NO: 75429.

The probes having the nucleotide sequence represented by SEQ ID NOS: 83818, 83819, 83820, 83821, 83822, 83823, 83824, 83825, 83826, 83827, 83828 were selected from the ORFs having the nucleotide sequence represented by SEQ ID NO: 75430.

The probes having the nucleotide sequence represented by SEQ ID NOS: 83829, 83830, 83831, 83832, 83833, 83834, 83835, 83836, 83837, 83838, 83839 were selected from the ORFs having the nucleotide sequence represented by SEQ ID NO: 75431.

The probes having the nucleotide sequence represented by SEQ ID NOS: 83840, 83841, 83842, 83843, 83844, 83845, 83846, 83847, 83848, 83849, 83850 were selected from the ORFs having the nucleotide sequence represented by SEQ ID NO: 75432.

The probes having the nucleotide sequence represented by SEQ ID NOS: 83851, 83852, 83853, 83854, 83855, 83856, 83857, 83858, 83859, 83860, 83861 were selected from the ORFs having the nucleotide sequence represented by SEQ ID NO: 75433.

The probes having the nucleotide sequence represented by SEQ ID NOS: 83862, 83863, 83864, 83865, 83866, 83867, 83868, 83869, 83870, 83871, 83872 were selected from the ORFs having the nucleotide sequence represented by SEQ ID NO: 75434.

The probes having the nucleotide sequence represented by SEQ ID NOS: 83873, 83874, 83875, 83876, 83877, 83878, 83879, 83880, 83881, 83882, 83883 were selected from the ORFs having the nucleotide sequence represented by SEQ ID NO: 75435.

The probes having the nucleotide sequence represented by SEQ ID NOS: 83884, 83885, 83886, 83887, 83888, 83889, 83890, 83891 were selected from the ORFs having the nucleotide sequence represented by SEQ ID NO: 75436.

The probes having the nucleotide sequence represented by SEQ ID NOS: 83892, 83893, 83894, 83895, 83896, 83897, 83898, 83899, 83900, 83901, 83902 were selected from the ORFs having the nucleotide sequence represented by SEQ ID NO: 75437.

The probes having the nucleotide sequence represented by SEQ ID NOS: 83903, 83904, 83905, 83906, 83907, 83908, 83909, 83910, 83911, 83912, 83913 were selected from the ORFs having the nucleotide sequence represented by SEQ ID NO: 75438.

The probes having the nucleotide sequence represented by SEQ ID NOS: 83914, 83915, 83916, 83917, 83918, 83919, 83920, 83921, 83922, 83923, 83924 were selected from the ORFs having the nucleotide sequence represented by SEQ ID NO: 75439.

The probes having the nucleotide sequence represented by SEQ ID NOS: 83925, 83926, 83927, 83928, 83929, 83930, 83931, 83932, 83933, 83934, 83935 were selected from the ORFs having the nucleotide sequence represented by SEQ ID NO: 75440.

The probes having the nucleotide sequence represented by SEQ ID NOS: 83936, 83937, 83938, 83939, 83940, 83941, 83942, 83943, 83944, 83945, 83946 were selected from the ORFs having the nucleotide sequence represented by SEQ ID NO: 75441.

The probes having the nucleotide sequence represented by SEQ ID NOS: 83947, 83948, 83949, 83950, 83951, 83952, 83953, 83954, 83955, 83956, 83957 were selected from the ORFs having the nucleotide sequence represented by SEQ ID NO: 75442.

The probes having the nucleotide sequence represented by SEQ ID NOS: 83958, 83959, 83960, 83961, 83962, 83963, 83964, 83965, 83966, 83967, 83968 were selected from the ORFs having the nucleotide sequence represented by SEQ ID NO: 75443.

The probes having the nucleotide sequence represented by SEQ ID NOS: 83969, 83970, 83971, 83972, 83973, 83974, 83975, 83976, 83977, 83978, 83979 were selected from the ORFs having the nucleotide sequence represented by SEQ ID NO: 75444.

The probes having the nucleotide sequence represented by SEQ ID NOS: 83980, 83981, 83982, 83983, 83984, 83985, 83986, 83987, 83988, 83989, 83990 were selected from the ORFs having the nucleotide sequence represented by SEQ ID NO: 75445.

The probes having the nucleotide sequence represented by SEQ ID NOS: 83991, 83992, 83993, 83994, 83995, 83996, 83997, 83998, 83999, 84000, 84001 were selected from the ORFs having the nucleotide sequence represented by SEQ ID NO: 75446.

The probes having the nucleotide sequence represented by SEQ ID NOS: 84002, 84003, 84004, 84005, 84006, 84007, 84008, 84009, 84010, 84011, 84012 were selected from the ORFs having the nucleotide sequence represented by SEQ ID NO: 75447.

The probes having the nucleotide sequence represented by SEQ ID NOS: 84013, 84014, 84015, 84016, 84017, 84018, 84019, 84020, 84021, 84022, 84023 were selected from the ORFs having the nucleotide sequence represented by SEQ ID NO: 75448.

The probes having the nucleotide sequence represented by SEQ ID NOS: 84024, 84025, 84026, 84027, 84028, 84029, 84030, 84031, 84032, 84033, 84034 were selected from the ORFs having the nucleotide sequence represented by SEQ ID NO: 75449.

The probes having the nucleotide sequence represented by SEQ ID NOS: 84035, 84036, 84037, 84038, 84039, 84040, 84041, 84042, 84043, 84044, 84045 were selected from the ORFs having the nucleotide sequence represented by SEQ ID NO: 75450.

The probes having the nucleotide sequence represented by SEQ ID NOS: 84046, 84047, 84048, 84049, 84050, 84051, 84052, 84053, 84054, 84055, 84056 were selected from the ORFs having the nucleotide sequence represented by SEQ ID NO: 75451.

The probes having the nucleotide sequence represented by SEQ ID NOS: 84057, 84058, 84059, 84060, 84061, 84062, 84063, 84064, 84065, 84066, 84067 were selected from the ORFs having the nucleotide sequence represented by SEQ ID NO: 75452.

The probes having the nucleotide sequence represented by SEQ ID NOS: 84068, 84069, 84070, 84071, 84072, 84073, 84074, 84075, 84076, 84077, 84078 were selected from the ORFs having the nucleotide sequence represented by SEQ ID NO: 75453.

The probes having the nucleotide sequence represented by SEQ ID NOS: 84079, 84080, 84081, 84082, 84083, 84084, 84085, 84086, 84087, 84088, 84089 were selected from the ORFs having the nucleotide sequence represented by SEQ ID NO: 75454.

The probes having the nucleotide sequence represented by SEQ ID NOS: 84090, 84091, 84092, 84093, 84094, 84095, 84096, 84097, 84098, 84099, 84100 were selected from the ORFs having the nucleotide sequence represented by SEQ ID NO: 75455.

The probes having the nucleotide sequence represented by SEQ ID NOS: 84101, 84102, 84103, 84104, 84105, 84106, 84107, 84108, 84109, 84110, 84111 were selected from the ORFs having the nucleotide sequence represented by SEQ ID NO: 75456.

The probes having the nucleotide sequence represented by SEQ ID NOS: 84112, 84113, 84114, 84115, 84116, 84117, 84118, 84119, 84120, 84121, 84122 were selected from the ORFs having the nucleotide sequence represented by SEQ ID NO: 75457.

The probes having the nucleotide sequence represented by SEQ ID NOS: 84123, 84124, 84125, 84126, 84127, 84128, 84129, 84130, 84131, 84132, 84133 were selected from the ORFs having the nucleotide sequence represented by SEQ ID NO: 75458.

The probes having the nucleotide sequence represented by SEQ ID NOS: 84134, 84135, 84136, 84137, 84138, 84139, 84140, 84141, 84142, 84143, 84144 were selected from the ORFs having the nucleotide sequence represented by SEQ ID NO: 75459.

The probes having the nucleotide sequence represented by SEQ ID NOS: 84145, 84146, 84147, 84148, 84149, 84150, 84151, 84152, 84153, 84154, 84155 were selected from the ORFs having the nucleotide sequence represented by SEQ ID NO: 75460.

The probes having the nucleotide sequence represented by SEQ ID NOS: 84156, 84157, 84158, 84159, 84160, 84161, 84162, 84163, 84164, 84165, 84166 were selected from the ORFs having the nucleotide sequence represented by SEQ ID NO: 75461.

The probes having the nucleotide sequence represented by SEQ ID NOS: 84167, 84168, 84169, 84170, 84171, 84172, 84173, 84174, 84175, 84176, 84177 were selected from the ORFs having the nucleotide sequence represented by SEQ ID NO: 75462.

The probes having the nucleotide sequence represented by SEQ ID NOS: 84178, 84179, 84180, 84181, 84182, 84183, 84184, 84185, 84186, 84187, 84188 were selected from the ORFs having the nucleotide sequence represented by SEQ ID NO: 75463.

The probes having the nucleotide sequence represented by SEQ ID NOS: 84189, 84190, 84191, 84192, 84193, 84194, 84195, 84196, 84197, 84198 were selected from the ORFs having the nucleotide sequence represented by SEQ ID NO: 75464.

The probes having the nucleotide sequence represented by SEQ ID NOS: 84199, 84200, 84201, 84202, 84203, 84204, 84205, 84206, 84207, 84208, 84209 were selected from the ORFs having the nucleotide sequence represented by SEQ ID NO: 75465.

The probes having the nucleotide sequence represented by SEQ ID NOS: 84210, 84211, 84212, 84213, 84214, 84215, 84216, 84217, 84218, 84219, 84220 were selected from the ORFs having the nucleotide sequence represented by SEQ ID NO: 75466.

The probes having the nucleotide sequence represented by SEQ ID NOS: 84221, 84222, 84223, 84224, 84225, 84226, 84227, 84228, 84229, 84230, 84231 were selected from the ORFs having the nucleotide sequence represented by SEQ ID NO: 75467.

The probes having the nucleotide sequence represented by SEQ ID NOS: 84232, 84233, 84234, 84235, 84236, 84237, 84238, 84239, 84240, 84241, 84242 were selected from the ORFs having the nucleotide sequence represented by SEQ ID NO: 75468.

The probes having the nucleotide sequence represented by SEQ ID NOS: 84243, 84244, 84245, 84246, 84247, 84248, 84249, 84250, 84251, 84252, 84253 were selected from the ORFs having the nucleotide sequence represented by SEQ ID NO: 75469.

The probes having the nucleotide sequence represented by SEQ ID NOS: 84254, 84255, 84256, 84257, 84258, 84259, 84260, 84261, 84262, 84263, 84264 were selected from the ORFs having the nucleotide sequence represented by SEQ ID NO: 75470.

The probes having the nucleotide sequence represented by SEQ ID NOS: 84265, 84266, 84267, 84268, 84269, 84270, 84271, 84272, 84273, 84274, 84275 were selected from the ORFs having the nucleotide sequence represented by SEQ ID NO: 75471.

The probes having the nucleotide sequence represented by SEQ ID NOS: 84276, 84277, 84278, 84279, 84280, 84281, 84282, 84283, 84284, 84285, 84286 were selected from the ORFs having the nucleotide sequence represented by SEQ ID NO: 75472.

The probes having the nucleotide sequence represented by SEQ ID NOS: 84287, 84288, 84289, 84290, 84291, 84292, 84293, 84294, 84295, 84296, 84297 were selected from the ORFs having the nucleotide sequence represented by SEQ ID NO: 75473.

The probes having the nucleotide sequence represented by SEQ ID NOS: 84298, 84299, 84300, 84301, 84302, 84303, 84304, 84305, 84306, 84307, 84308 were selected from the ORFs having the nucleotide sequence represented by SEQ ID NO: 75474.

The probes having the nucleotide sequence represented by SEQ ID NOS: 84309, 84310, 84311, 84312, 84313, 84314, 84315, 84316, 84317, 84318, 84319 were selected from the ORFs having the nucleotide sequence represented by SEQ ID NO: 75475.

The probes having the nucleotide sequence represented by SEQ ID NOS: 84320, 84321, 84322, 84323, 84324, 84325, 84326, 84327, 84328, 84329, 84330 were selected from the ORFs having the nucleotide sequence represented by SEQ ID NO: 75476.

The probes having the nucleotide sequence represented by SEQ ID NOS: 84331, 84332, 84333, 84334, 84335, 84336, 84337, 84338, 84339, 84340, 84341 were selected from the ORFs having the nucleotide sequence represented by SEQ ID NO: 75477.

The probes having the nucleotide sequence represented by SEQ ID NOS: 84342, 84343, 84344, 84345, 84346, 84347, 84348, 84349, 84350, 84351, 84352 were selected from the ORFs having the nucleotide sequence represented by SEQ ID NO: 75478.

The probes having the nucleotide sequence represented by SEQ ID NOS: 84353, 84354, 84355, 84356, 84357, 84358, 84359, 84360, 84361, 84362, 84363 were selected from the ORFs having the nucleotide sequence represented by SEQ ID NO: 75479.

The probes having the nucleotide sequence represented by SEQ ID NOS: 84364, 84365, 84366, 84367, 84368, 84369, 84370, 84371, 84372, 84373, 84374 were selected from the ORFs having the nucleotide sequence represented by SEQ ID NO: 75480.

The probes having the nucleotide sequence represented by SEQ ID NOS: 84375, 84376, 84377, 84378, 84379, 84380, 84381, 84382, 84383, 84384, 84385 were selected from the ORFs having the nucleotide sequence represented by SEQ ID NO: 75481.

The probes having the nucleotide sequence represented by SEQ ID NOS: 84386, 84387, 84388, 84389, 84390, 84391, 84392, 84393, 84394, 84395, 84396 were selected from the ORFs having the nucleotide sequence represented by SEQ ID NO: 75482.

The probes having the nucleotide sequence represented by SEQ ID NOS: 84397, 84398, 84399, 84400, 84401, 84402, 84403, 84404, 84405, 84406, 84407 were selected from the ORFs having the nucleotide sequence represented by SEQ ID NO: 75483.

The probes having the nucleotide sequence represented by SEQ ID NOS: 84408, 84409, 84410, 84411, 84412, 84413, 84414, 84415, 84416, 84417, 84418 were selected from the ORFs having the nucleotide sequence represented by SEQ ID NO: 75484.

The probes having the nucleotide sequence represented by SEQ ID NOS: 84419, 84420, 84421, 84422, 84423, 84424, 84425, 84426, 84427, 84428, 84429 were selected from the ORFs having the nucleotide sequence represented by SEQ ID NO: 75485.

The probes having the nucleotide sequence represented by SEQ ID NOS:.84430, 84431, 84432, 84433, 84434, 84435, 84436, 84437, 84438, 84439, 84440 were selected from the ORFs having the nucleotide sequence represented by SEQ ID NO: 75486.

The probes having the nucleotide sequence represented by SEQ ID NOS: 84441, 84442, 84443, 84444, 84445, 84446, 84447, 84448, 84449, 84450, 84451 were selected from the ORFs having the nucleotide sequence represented by SEQ ID NO: 75487.

The probes having the nucleotide sequence represented by SEQ ID NOS: 84452, 84453, 84454, 84455, 84456, 84457, 84458, 84459, 84460, 84461, 84462 were selected from the ORFs having the nucleotide sequence represented by SEQ ID NO: 75488.

The probes having the nucleotide sequence represented by SEQ ID NOS: 84463, 84464, 84465, 84466, 84467, 84468, 84469, 84470, 84471, 84472, 84473 were selected from the ORFs having the nucleotide sequence represented by SEQ ID NO: 75489.

The probes having the nucleotide sequence represented by SEQ ID NOS: 84474, 84475, 84476, 84477, 84478, 84479, 84480, 84481, 84482, 84483, 84484 were selected from the ORFs having the nucleotide sequence represented by SEQ ID NO: 75490.

The probes having the nucleotide sequence represented by SEQ ID NOS: 84485, 84486, 84487, 84488, 84489, 84490, 84491, 84492, 84493, 84494, 84495 were selected from the ORFs having the nucleotide sequence represented by SEQ ID NO: 75491.

The probes having the nucleotide sequence represented by SEQ ID NOS: 84496, 84497, 84498, 84499, 84500, 84501, 84502, 84503, 84504, 84505, 84506 were selected from the ORFs having the nucleotide sequence represented by SEQ ID NO: 75492.

The probes having the nucleotide sequence represented by SEQ ID NOS: 84507, 84508, 84509, 84510, 84511, 84512, 84513, 84514, 84515, 84516, 84517 were selected from the ORFs having the nucleotide sequence represented by SEQ ID NO: 75493.

The probes having the nucleotide sequence represented by SEQ ID NOS: 84518, 84519, 84520, 84521, 84522, 84523, 84524, 84525, 84526, 84527, 84528 were selected from the ORFs having the nucleotide sequence represented by SEQ ID NO: 75494.

The probes having the nucleotide sequence represented by SEQ ID NOS: 84529, 84530, 84531, 84532, 84533, 84534, 84535, 84536, 84537, 84538, 84539 were selected from the ORFs having the nucleotide sequence represented by SEQ ID NO: 75495.

The probes having the nucleotide sequence represented by SEQ ID NOS: 84540, 84541, 84542, 84543, 84544, 84545, 84546, 84547, 84548, 84549, 84550 were selected from the ORFs having the nucleotide sequence represented by SEQ ID NO: 75496.

The probes having the nucleotide sequence represented by SEQ ID NOS: 84551, 84552, 84553, 84554, 84555, 84556, 84557, 84558, 84559, 84560, 84561 were selected from the ORFs having the nucleotide sequence represented by SEQ ID NO: 75497.

The probes having the nucleotide sequence represented by SEQ ID NOS: 84562, 84563, 84564, 84565, 84566, 84567, 84568, 84569, 84570, 84571, 84572 were selected from the ORFs having the nucleotide sequence represented by SEQ ID NO: 75498.

The probes having the nucleotide sequence represented by SEQ ID NOS: 84573, 84574, 84575, 84576, 84577, 84578, 84579, 84580, 84581, 84582, 84583 were selected from the ORFs having the nucleotide sequence represented by SEQ ID NO: 75499.

The probes having the nucleotide sequence represented by SEQ ID NOS: 84584, 84585, 84586, 84587, 84588, 84589, 84590, 84591, 84592, 84593, 84594 were selected from the ORFs having the nucleotide sequence represented by SEQ ID NO: 75500.

The probes having the nucleotide sequence represented by SEQ ID NOS: 84595, 84596, 84597, 84598, 84599, 84600, 84601, 84602, 84603, 84604, 84605 were selected from the ORFs having the nucleotide sequence represented by SEQ ID NO: 75501.

The probes having the nucleotide sequence represented by SEQ ID NOS: 84606, 84607, 84608, 84609, 84610, 84611, 84612, 84613, 84614, 84615, 84616 were selected from the ORFs having the nucleotide sequence represented by SEQ ID NO: 75502.

The probes having the nucleotide sequence represented by SEQ ID NOS: 84617, 84618, 84619, 84620, 84621, 84622, 84623, 84624, 84625, 84626, 84627 were selected from the ORFs having the nucleotide sequence represented by SEQ ID NO: 75503.

The probes having the nucleotide sequence represented by SEQ ID NOS: 84628, 84629, 84630, 84631, 84632, 84633, 84634, 84635, 84636, 84637, 84638 were selected from the ORFs having the nucleotide sequence represented by SEQ ID NO: 75504.

The probes having the nucleotide sequence represented by SEQ ID NOS: 84639, 84640, 84641, 84642, 84643, 84644, 84645, 84646, 84647, 84648, 84649 were selected from the ORFs having the nucleotide sequence represented by SEQ ID NO: 75505.

The probes having the nucleotide sequence represented by SEQ ID NOS: 84650, 84651, 84652, 84653, 84654, 84655, 84656, 84657, 84658, 84659, 84660 were selected from the ORFs having the nucleotide sequence represented by SEQ ID NO: 75506.

The probes having the nucleotide sequence represented by SEQ ID NOS: 84661, 84662, 84663, 84664, 84665, 84666, 84667, 84668, 84669, 84670, 84671 were selected from the ORFs having the nucleotide sequence represented by SEQ ID NO: 75507.

The probes having the nucleotide sequence represented by SEQ ID NOS: 84672, 84673, 84674, 84675, 84676, 84677, 84678, 84679, 84680, 84681, 84682 were selected from the ORFs having the nucleotide sequence represented by SEQ ID NO: 75508.

The probes having the nucleotide sequence represented by SEQ ID NOS: 84683, 84684, 84685, 84686, 84687, 84688, 84689, 84690, 84691, 84692, 84693 were selected from the ORFs having the nucleotide sequence represented by SEQ ID NO: 75509.

The probes having the nucleotide sequence represented by SEQ ID NOS: 84694, 84695, 84696, 84697, 84698, 84699, 84700, 84701, 84702, 84703, 84704 were selected from the ORFs having the nucleotide sequence represented by SEQ ID NO: 75510.

The probes having the nucleotide sequence represented by SEQ ID NOS: 84705, 84706, 84707, 84708, 84709, 84710, 84711, 84712, 84713, 84714, 84715 were selected from the ORFs having the nucleotide sequence represented by SEQ ID NO: 75511.

The probes having the nucleotide sequence represented by SEQ ID NOS: 84716, 84717, 84718, 84719, 84720, 84721, 84722, 84723, 84724, 84725, 84726 were selected from the ORFs having the nucleotide sequence represented by SEQ ID NO: 75512.

The probes having the nucleotide sequence represented by SEQ ID NOS: 84727, 84728, 84729, 84730, 84731, 84732, 84733, 84734, 84735, 84736, 84737 were selected from the ORFs having the nucleotide sequence represented by SEQ ID NO: 75513.

The probes having the nucleotide sequence represented by SEQ ID NOS: 84738, 84739, 84740, 84741, 84742, 84743, 84744, 84745, 84746, 84747, 84748 were selected from the ORFs having the nucleotide sequence represented by SEQ ID NO: 75514.

The probes having the nucleotide sequence represented by SEQ ID NOS: 84749, 84750, 84751, 84752, 84753, 84754, 84755, 84756, 84757, 84758, 84759 were selected from the ORFs having the nucleotide sequence represented by SEQ ID NO: 75515.

The probes having the nucleotide sequence represented by SEQ ID NOS: 84760, 84761, 84762, 84763, 84764, 84765, 84766, 84767, 84768, 84769, 84770 were selected from the ORFs having the nucleotide sequence represented by SEQ ID NO: 75516.

The probes having the nucleotide sequence represented by SEQ ID NOS: 84771, 84772, 84773, 84774, 84775, 84776, 84777, 84778, 84779, 84780, 84781 were selected from the ORFs having the nucleotide sequence represented by SEQ ID NO: 75517.

The probes having the nucleotide sequence represented by SEQ ID NOS: 84782, 84783, 84784, 84785, 84786, 84787, 84788, 84789, 84790, 84791, 84792 were selected from the ORFs having the nucleotide sequence represented by SEQ ID NO: 75518.

The probes having the nucleotide sequence represented by SEQ ID NOS: 84793, 84794, 84795, 84796, 84797, 84798, 84799, 84800, 84801, 84802, 84803 were selected from the ORFs having the nucleotide sequence represented by SEQ ID NO: 75519.

The probes having the nucleotide sequence represented by SEQ ID NOS: 84804, 84805, 84806, 84807, 84808, 84809, 84810, 84811, 84812, 84813, 84814 were selected from the ORFs having the nucleotide sequence represented by SEQ ID NO: 75520.

The probes having the nucleotide sequence represented by SEQ ID NOS: 84815, 84816, 84817, 84818, 84819, 84820, 84821, 84822, 84823, 84824, 84825 were selected from the ORFs having the nucleotide sequence represented by SEQ ID NO: 75521.

The probes having the nucleotide sequence represented by SEQ ID NOS: 84826, 84827, 84828, 84829, 84830, 84831, 84832, 84833, 84834, 84835, 84836 were selected from the ORFs having the nucleotide sequence represented by SEQ ID NO: 75522.

The probes having the nucleotide sequence represented by SEQ ID NOS: 84837, 84838, 84839, 84840, 84841, 84842, 84843, 84844, 84845, 84846, 84847 were selected from the ORFs having the nucleotide sequence represented by SEQ ID NO: 75523.

The probes having the nucleotide sequence represented by SEQ ID NOS: 84848, 84849, 84850, 84851, 84852, 84853, 84854, 84855, 84856, 84857, 84858 were selected from the ORFs having the nucleotide sequence represented by SEQ ID NO: 75524.

The probes having the nucleotide sequence represented by SEQ ID NOS: 84859, 84860, 84861, 84862, 84863, 84864, 84865, 84866, 84867, 84868, 84869 were selected from the ORFs having the nucleotide sequence represented by SEQ ID NO: 75525.

The probes having the nucleotide sequence represented by SEQ ID NOS: 84870, 84871, 84872, 84873, 84874, 84875, 84876, 84877, 84878, 84879, 84880 were selected from the ORFs having the nucleotide sequence represented by SEQ ID NO: 75526.

The probes having the nucleotide sequence represented by SEQ ID NOS: 84881, 84882, 84883, 84884, 84885, 84886, 84887, 84888, 84889, 84890, 84891 were selected from the ORFs having the nucleotide sequence represented by SEQ ID NO: 75527.

The probes having the nucleotide sequence represented by SEQ ID NOS: 84892, 84893, 84894, 84895, 84896, 84897, 84898, 84899, 84900, 84901, 84902 were selected from the ORFs having the nucleotide sequence represented by SEQ ID NO: 75528.

The probes having the nucleotide sequence represented by SEQ ID NOS: 84903, 84904, 84905, 84906, 84907, 84908, 84909, 84910, 84911, 84912, 84913 were selected from the ORFs having the nucleotide sequence represented by SEQ ID NO: 75529.

The probes having the nucleotide sequence represented by SEQ ID NOS: 84914, 84915, 84916, 84917, 84918, 84919, 84920, 84921, 84922, 84923, 84924 were selected from the ORFs having the nucleotide sequence represented by SEQ ID NO: 75530.

The probes having the nucleotide sequence represented by SEQ ID NOS: 84925, 84926, 84927, 84928, 84929, 84930, 84931, 84932, 84933, 84934, 84935 were selected from the ORFs having the nucleotide sequence represented by SEQ ID NO: 75531.

The probes having the nucleotide sequence represented by SEQ ID NOS: 84936, 84937, 84938, 84939, 84940, 84941, 84942, 84943, 84944, 84945, 84946 were selected from the ORFs having the nucleotide sequence represented by SEQ ID NO: 75532.

The probes having the nucleotide sequence represented by SEQ ID NOS: 84947, 84948, 84949, 84950, 84951, 84952, 84953, 84954, 84955, 84956, 84957 were selected from the ORFs having the nucleotide sequence represented by SEQ ID NO: 75533.

The probes having the nucleotide sequence represented by SEQ ID NOS: 84958, 84959, 84960, 84961, 84962, 84963, 84964, 84965, 84966, 84967, 84968 were selected from the ORFs having the nucleotide sequence represented by SEQ ID NO: 75534.

The probes having the nucleotide sequence represented by SEQ ID NOS: 84969, 84970, 84971, 84972, 84973, 84974, 84975, 84976, 84977, 84978, 84979 were selected from the ORFs having the nucleotide sequence represented by SEQ ID NO: 75535.

The probes having the nucleotide sequence represented by SEQ ID NOS: 84980, 84981, 84982, 84983, 84984, 84985, 84986, 84987, 84988, 84989, 84990 were selected from the ORFs having the nucleotide sequence represented by SEQ ID NO: 75536.

The probes having the nucleotide sequence represented by SEQ ID NOS: 84991, 84992, 84993, 84994, 84995, 84996, 84997, 84998, 84999, 85000, 85001 were selected from the ORFs having the nucleotide sequence represented by SEQ ID NO: 75537.

The probes having the nucleotide sequence represented by SEQ ID NOS: 85002, 85003, 85004, 85005, 85006, 85007, 85008, 85009, 85010, 85011, 85012 were selected from the ORFs having the nucleotide sequence represented by SEQ ID NO: 75538.

The probes having the nucleotide sequence represented by SEQ ID NOS: 85013, 85014, 85015, 85016, 85017, 85018, 85019, 85020, 85021, 85022, 85023 were selected from the ORFs having the nucleotide sequence represented by SEQ ID NO: 75539.

The probes having the nucleotide sequence represented by SEQ ID NOS: 85024, 85025, 85026, 85027, 85028, 85029, 85030, 85031, 85032, 85033, 85034 were selected from the ORFs having the nucleotide sequence represented by SEQ ID NO: 75540.

The probes having the nucleotide sequence represented by SEQ ID NOS: 85035, 85036, 85037, 85038, 85039, 85040, 85041, 85042, 85043, 85044, 85045 were selected from the ORFs having the nucleotide sequence represented by SEQ ID NO: 75541.

The probes having the nucleotide sequence represented by SEQ ID NOS: 85046, 85047, 85048, 85049, 85050, 85051, 85052, 85053, 85054, 85055, 85056 were selected from the ORFs having the nucleotide sequence represented by SEQ ID NO: 75542.

The probes having the nucleotide sequence represented by SEQ ID NOS: 85057, 85058, 85059, 85060, 85061, 85062, 85063, 85064, 85065, 85066, 85067 were selected from the ORFs having the nucleotide sequence represented by SEQ ID NO: 75543.

The probes having the nucleotide sequence represented by SEQ ID NOS: 85068, 85069, 85070, 85071, 85072, 85073,

85074, 85075, 85076, 85077, 85078 were selected from the ORFs having the nucleotide sequence represented by SEQ ID NO: 75544.

The probes having the nucleotide sequence represented by SEQ ID NOS: 85079, 85080, 85081, 85082, 85083, 85084, 85085, 85086, 85087, 85088, 85089 were selected from the ORFs having the nucleotide sequence represented by SEQ ID NO: 75545.

The probes having the nucleotide sequence represented by SEQ ID NOS: 85090, 85091, 85092, 85093, 85094, 85095, 85096, 85097, 85098, 85099, 85100 were selected from the ORFs having the nucleotide sequence represented by SEQ ID NO: 75546.

The probes having the nucleotide sequence represented by SEQ ID NOS: 85101, 85102, 85103, 85104, 85105, 85106, 85107, 85108, 85109, 85110, 85111 were selected from the ORFs having the nucleotide sequence represented by SEQ ID NO: 75547.

The probes having the nucleotide sequence represented by SEQ ID NOS: 85112, 85113, 85114, 85115, 85116, 85117, 85118, 85119, 85120, 85121, 85122 were selected from the ORFs having the nucleotide sequence represented by SEQ ID NO: 75548.

The probes having the nucleotide sequence represented by SEQ ID NOS: 85123, 85124, 85125, 85126, 85127, 85128, 85129, 85130, 85131, 85132, 85133 were selected from the ORFs having the nucleotide sequence represented by SEQ ID NO: 75549.

The probes having the nucleotide sequence represented by SEQ ID NOS: 85134, 85135, 85136, 85137, 85138, 85139, 85140, 85141, 85142, 85143, 85144 were selected from the ORFs having the nucleotide sequence represented by SEQ ID NO: 75550.

The probes having the nucleotide sequence represented by SEQ ID NOS: 85145, 85146, 85147, 85148, 85149, 85150, 85151, 85152, 85153, 85154, 85155 were selected from the ORFs having the nucleotide sequence represented by SEQ ID NO: 75551.

The probes having the nucleotide sequence represented by SEQ ID NOS: 85156, 85157, 85158, 85159, 85160, 85161, 85162, 85163,. 85164, 85165, 85166 were selected from the ORFs having the nucleotide sequence represented by SEQ ID NO: 75552.

The probes having the nucleotide sequence represented by SEQ ID NOS: 85167, 85168, 85169, 85170, 85171, 85172, 85173, 85174, 85175, 85176, 85177 were selected from the ORFs having the nucleotide sequence represented by SEQ ID NO: 75553.

The probes having the nucleotide sequence represented by SEQ ID NOS: 85178, 85179, 85180, 85181, 85182, 85183, 85184, 85185, 85186, 85187, 85188 were selected from the ORFs having the nucleotide sequence represented by SEQ ID NO: 75554.

The probes having the nucleotide sequence represented by SEQ ID NOS: 85189, 85190, 85191, 85192, 85193, 85194, 85195, 85196, 85197, 85198, 85199 were selected from the ORFs having the nucleotide sequence represented by SEQ ID NO: 75555.

The probes having the nucleotide sequence represented by SEQ ID NOS: 85200, 85201, 85202, 85203, 85204, 85205, 85206, 85207, 85208, 85209, 85210 were selected from the ORFs having the nucleotide sequence represented by SEQ ID NO: 75556.

The probes having the nucleotide sequence represented by SEQ ID NOS: 85211, 85212, 85213, 85214, 85215, 85216, 85217, 85218, 85219, 85220, 85221 were selected from the ORFs having the nucleotide sequence represented by SEQ ID NO: 75557.

The probes having the nucleotide sequence represented by SEQ ID NOS: 85222, 85223, 85224, 85225, 85226, 85227, 85228, 85229, 85230, 85231, 85232 were selected from the ORFs having the nucleotide sequence represented by SEQ ID NO: 75558.

The probes having the nucleotide sequence represented by SEQ ID NOS: 85233, 85234, 85235, 85236, 85237, 85238, 85239, 85240, 85241, 85242, 85243 were selected from the ORFs having the nucleotide sequence represented by SEQ ID NO: 75559.

The probes having the nucleotide sequence represented by SEQ ID NOS: 85244, 85245, 85246, 85247, 85248, 85249, 85250, 85251, 85252, 85253, 85254 were selected from the ORFs having the nucleotide sequence represented by SEQ ID NO: 75560.

The probes having the nucleotide sequence represented by SEQ ID NOS: 85255, 85256, 85257, 85258, 85259, 85260, 85261, 85262, 85263, 85264, 85265 were selected from the ORFs having the nucleotide sequence represented by SEQ ID NO: 75561.

The probes having the nucleotide sequence represented by SEQ ID NOS: 85266, 85267, 85268, 85269, 85270, 85271, 85272, 85273, 85274, 85275, 85276 were selected from the ORFs having the nucleotide sequence represented by SEQ ID NO: 75562.

The probes having the nucleotide sequence represented by SEQ ID NOS: 85277, 85278, 85279, 85280, 85281, 85282, 85283, 85284, 85285, 85286, 85287 were selected from the ORFs having the nucleotide sequence represented by SEQ ID NO: 75563.

The probes having the nucleotide sequence represented by SEQ ID NOS: 85288, 85289, 85290, 85291, 85292, 85293, 85294, 85295, 85296, 85297, 85298 were selected from the ORFs having the nucleotide sequence represented by SEQ ID NO: 75564.

The probes having the nucleotide sequence represented by SEQ ID NOS: 85299, 85300, 85301, 85302, 85303, 85304, 85305, 85306, 85307, 85308, 85309 were selected from the ORFs having the nucleotide sequence represented by SEQ ID NO: 75565.

The probes having the nucleotide sequence represented by SEQ ID NOS: 85310, 85311, 85312, 85313, 85314, 85315, 85316, 85317, 85318, 85319, 85320 were selected from the ORFs having the nucleotide sequence represented by SEQ ID NO: 75566.

The probes having the nucleotide sequence represented by SEQ ID NOS: 85321, 85322, 85323, 85324, 85325, 85326, 85327, 85328, 85329, 85330, 85331 were selected from the ORFs having the nucleotide sequence represented by SEQ ID NO: 75567.

The probes having the nucleotide sequence represented by SEQ ID NOS: 85332, 85333, 85334, 85335, 85336, 85337, 85338, 85339, 85340, 85341, 85342 were selected from the ORFs having the nucleotide sequence represented by SEQ ID NO: 75568.

The probes having the nucleotide sequence represented by SEQ ID NOS: 85343,. 85344, 85345, 85346, 85347, 85348, 85349, 85350, 85351, 85352, 85353 were selected from the ORFs having the nucleotide sequence represented by SEQ ID NO: 75569.

The probes having the nucleotide sequence represented by SEQ ID NOS: 85354, 85355, 85356, 85357, 85358, 85359, 85360, 85361, 85362, 85363, 85364 were selected from the ORFs having the nucleotide sequence represented by SEQ ID NO: 75570.

The probes having the nucleotide sequence represented by SEQ ID NOS: 85365, 85366, 85367, 85368, 85369, 85370, 85371, 85372, 85373, 85374, 85375 were selected from the ORFs having the nucleotide sequence represented by SEQ ID NO: 75571.

The probes having the nucleotide sequence represented by SEQ ID NOS: 85376, 85377, 85378, 85379, 85380, 85381, 85382, 85383, 85384, 85385, 85386 were selected from the ORFs having the nucleotide sequence represented by SEQ ID NO: 75572.

The probes having the nucleotide sequence represented by SEQ ID NOS: 85387, 85388, 85389, 85390, 85391, 85392, 85393, 85394, 85395, 85396, 85397 were selected from the ORFs having the nucleotide sequence represented by SEQ ID NO: 75573.

The probes having the nucleotide sequence represented by SEQ ID NOS: 85398, 85399, 85400, 85401, 85402, 85403, 85404, 85405, 85406, 85407, 85408 were selected from the ORFs having the nucleotide sequence represented by SEQ ID NO: 75574.

The probes having the nucleotide sequence represented by SEQ ID NOS: 85409, 85410, 85411, 85412, 85413, 85414, 85415, 85416, 85417, 85418, 85419 were selected from the ORFs having the nucleotide sequence represented by SEQ ID NO: 75575.

The probes having the nucleotide sequence represented by SEQ ID NOS: 85420, 85421, 85422, 85423, 85424, 85425, 85426, 85427, 85428, 85429, 85430 were selected from the ORFs having the nucleotide sequence represented by SEQ ID NO: 75576.

The probes having the nucleotide sequence represented by SEQ ID NOS: 85431, 85432, 85433, 85434, 85435, 85436, 85437, 85438, 85439, 85440, 85441 were selected from the ORFs having the nucleotide sequence represented by SEQ ID NO: 75577.

The probes having the nucleotide sequence represented by SEQ ID NOS: 85442, 85443, 85444, 85445, 85446, 85447, 85448, 85449, 85450, 85451, 85452 were selected from the ORFs having the nucleotide sequence represented by SEQ ID NO: 75578.

The probes having the nucleotide sequence represented by SEQ ID NOS: 85453, 85454, 85455, 85456, 85457, 85458, 85459, 85460, 85461, 85462, 85463 were selected from the ORFs having the nucleotide sequence represented by SEQ ID NO: 75579.

The probes having the nucleotide sequence represented by SEQ ID NOS: 85464, 85465., 85466, 85467, 85468, 85469, 85470, 85471, 85472, 85473, 85474 were selected from the ORFs having the nucleotide sequence represented by SEQ ID NO: 75580.

The probes having the nucleotide sequence represented by SEQ ID NOS: 85475, 85476, 85477, 85478, 85479, 85480, 85481, 85482, 85483, 85484, 85485 were selected from the ORFs having the nucleotide sequence represented by SEQ ID NO: 75581.

The probes having the nucleotide sequence represented by SEQ ID NOS: 85486, 85487, 85488, 85489, 85490, 85491, 85492, 85493, 85494, 85495, 85496 were selected from the ORFs having the nucleotide sequence represented by SEQ ID NO: 75582.

The probes having the nucleotide sequence represented by SEQ ID NOS: 85497, 85498, 85499, 85500, 85501, 85502, 85503, 85504, 85505, 85506, 85507 were selected from the ORFs having the nucleotide sequence represented by SEQ ID NO: 75583.

The probes having the nucleotide sequence represented by SEQ ID NOS: 85508, 85509, 85510, 85511, 85512, 85513, 85514, 85515, 85516, 85517, 85518 were selected from the ORFs having the nucleotide sequence represented by SEQ ID NO: 75584.

The probes having the nucleotide sequence represented by SEQ ID NOS: 85519, 85520, 85521, 85522, 85523, 85524, 85525, 85526, 85527, 85528, 85529 were selected from the ORFs having the nucleotide sequence represented by SEQ ID NO: 75585.

The probes having the nucleotide sequence represented by SEQ ID NOS: 85530, 85531, 85532, 85533, 85534, 85535, 85536, 85537, 85538, 85539, 85540 were selected from the ORFs having the nucleotide sequence represented by SEQ ID NO: 75586.

The probes having the nucleotide sequence represented by SEQ ID NOS: 85541, 85542, 85543, 85544, 85545, 85546, 85547, 85548, 85549, 85550, 85551 were selected from the ORFs having the nucleotide sequence represented by SEQ ID NO: 75587.

The probes having the nucleotide sequence represented by SEQ ID NOS: 85552, 85553, 85554, 85555, 85556, 85557, 85558, 85559, 85560, 85561, 85562 were selected from the ORFs having the nucleotide sequence represented by SEQ ID NO: 75588.

The probes having the nucleotide sequence represented by SEQ ID NOS: 85563, 85564, 85565, 85566, 85567, 85568, 85569, 85570, 85571, 85572, 85573 were selected from the ORFs having the nucleotide sequence represented by SEQ ID NO: 75589.

The probes having the nucleotide sequence represented by SEQ ID NOS: 85574, 85575, 85576, 85577, 85578, 85579, 85580, 85581, 85582, 85583, 85584 were selected from the ORFs having the nucleotide sequence represented by SEQ ID NO: 75590.

The probes having the nucleotide sequence represented by SEQ ID NOS: 85585, 85586, 85587, 85588, 85589, 85590, 85591, 85592, 85593, 85594, 85595 were selected from the ORFs having the nucleotide sequence represented by SEQ ID NO: 75591.

The probes having the nucleotide sequence represented by SEQ ID NOS: 85596, 85597, 85598, 85599, 85600, 85601, 85602, 85603, 85604, 85605, 85606 were selected from the ORFs having the nucleotide sequence represented by SEQ ID NO: 75592.

The probes having the nucleotide sequence represented by SEQ ID NOS: 85607, 85608, 85609, 85610, 85611, 85612, 85613, 85614, 85615, 85616, 85617 were selected from the ORFs having the nucleotide sequence represented by SEQ ID NO: 75593.

The probes having the nucleotide sequence represented by SEQ ID NOS: 85618, 85619, 85620, 85621, 85622, 85623, 85624, 85625, 85626, 85627, 85628 were selected from the ORFs having the nucleotide sequence represented by SEQ ID NO: 75594.

The probes having the nucleotide sequence represented by SEQ ID NOS: 85629, 85630, 85631, 85632, 85633, 85634, 85635, 85636, 85637, 85638, 85639 were selected from the ORFs having the nucleotide sequence represented by SEQ ID NO: 75595.

The probes having the nucleotide sequence represented by SEQ ID NOS: 85640, 85641, 85642, 85643, 85644, 85645, 85646, 85647, 85648, 85649, 85650 were selected from the ORFs having the nucleotide sequence represented by SEQ ID NO: 75596.

The probes having the nucleotide sequence represented by SEQ ID NOS: 85651, 85652, 85653, 85654, 85655, 85656, 85657, 85658, 85659, 85660, 85661 were selected from the ORFs having the nucleotide sequence represented by SEQ ID NO: 75597.

The probes having the nucleotide sequence represented by SEQ ID NOS: 85662, 85663, 85664, 85665, 85666, 85667, 85668, 85669, 85670, 85671, 85672 were selected from the ORFs having the nucleotide sequence represented by SEQ ID NO: 75598.

The probes having the nucleotide sequence represented by SEQ ID NOS: 85673, 85674, 85675, 85676, 85677, 85678, 85679, 85680, 85681, 85682, 85683 were selected from the ORFs having the nucleotide sequence represented by SEQ ID NO: 75599.

The probes having the nucleotide sequence represented by SEQ ID NOS: 85684, 85685, 85686, 85687, 85688, 85689, 85690, 85691, 85692, 85693, 85694 were selected from the ORFs having the nucleotide sequence represented by SEQ ID NO: 75600.

The probes having the nucleotide sequence represented by SEQ ID NOS: 85695, 85696, 85697, 85698, 85699, 85700, 85701, 85702, 85703, 85704, 85705 were selected from the ORFs having the nucleotide sequence represented by SEQ ID NO: 75601.

The probes having the nucleotide sequence represented by SEQ ID NOS: 85706, 85707, 85708, 85709, 85710, 85711, 85712, 85713, 85714, 85715, 85716 were selected from the ORFs having the nucleotide sequence represented by SEQ ID NO: 75602.

The probes having the nucleotide sequence represented by SEQ ID NOS: 85717, 85718, 85719, 85720, 85721, 85722, 85723, 85724, 85725, 85726, 85727 were selected from the ORFs having the nucleotide sequence represented by SEQ ID NO: 75603.

The probes having the nucleotide sequence represented by SEQ ID NOS: 85728, 85729, 85730, 85731, 85732, 85733, 85734, 85735, 85736, 85737, 85738 were selected from the ORFs having the nucleotide sequence represented by SEQ ID NO: 75604.

The probes having the nucleotide sequence represented by SEQ ID NOS: 85739, 85740, 85741, 85742, 85743, 85744, 85745, 85746, 85747, 85748, 85749 were selected from the ORFs having the nucleotide sequence represented by SEQ ID NO: 75605.

The probes having the nucleotide sequence represented by SEQ ID NOS: 85750, 85751, 85752, 85753, 85754, 85755, 85756, 85757, 85758, 85759, 85760, 85761, 85762, 85763, 85764, 85765, 85766, 85767, 85768, 85769, 85770, 85771 were selected from the ORFs having the nucleotide sequence represented by SEQ ID NO: 75606.

The probes having the nucleotide sequence represented by SEQ ID NOS: 85772, 85773, 85774, 85775, 85776, 85777, 85778, 85779, 85780, 85781, 85782 were selected from the ORFs having the nucleotide sequence represented by SEQ ID NO: 75607.

The probes having the nucleotide sequence represented by SEQ ID NOS: 85783, 85784, 85785, 85786, 85787, 85788, 85789, 85790, 85791, 85792, 85793 were selected from the ORFs having the nucleotide sequence represented by SEQ ID NO: 75608.

The probes having the nucleotide sequence represented by SEQ ID NOS: 85794, 85795, 85796, 85797, 85798, 85799, 85800, 85801, 85802, 85803, 85804 were selected from the ORFs having the nucleotide sequence represented by SEQ ID NO: 75609.

The probes having the nucleotide sequence represented by SEQ ID NOS: 85805, 85806, 85807, 85808, 85809, 85810, 85811, 85812, 85813, 85814, 85815 were selected from the ORFs having the nucleotide sequence represented by SEQ ID NO: 75610.

The probes having the nucleotide sequence represented by SEQ ID NOS: 85816, 85817, 85818, 85819, 85820, 85821, 85822, 85823, 85824, 85825, 85826 were selected from the ORFs having the nucleotide sequence represented by SEQ ID NO: 75611.

The probes having the nucleotide sequence represented by SEQ ID NOS: 85827, 85828, 85829, 85830, 85831, 85832, 85833, 85834, 85835, 85836, 85837 were selected from the ORFs having the nucleotide sequence represented by SEQ ID NO: 75612.

The probes having the nucleotide sequence represented by SEQ ID NOS: 85838, 85839, 85840, 85841, 85842, 85843, 85844, 85845, 85846, 85847, 85848 were selected from the ORFs having the nucleotide sequence represented by SEQ ID NO: 75613.

The probes having the nucleotide sequence represented by SEQ ID NOS: 85849, 85850, 85851, 85852, 85853, 85854, 85855, 85856, 85857, 85858, 85859 were selected from the ORFs having the nucleotide sequence represented by SEQ ID NO: 75614.

The probes having the nucleotide sequence represented by SEQ ID NOS: 85860, 85861, 85862, 85863, 85864, 85865, 85866, 85867, 85868, 85869, 85870 were selected from the ORFs having the nucleotide sequence represented by SEQ ID NO: 75615.

The probes having the nucleotide sequence represented by SEQ ID NOS: 85871, 85872, 85873, 85874, 85875, 85876, 85877, 85878, 85879, 85880, 85881 were selected from the ORFs having the nucleotide sequence represented by SEQ ID NO: 75616.

The probes having the nucleotide sequence represented by SEQ ID NOS: 85882, 85883, 85884, 85885, 85886, 85887, 85888, 85889, 85890, 85891, 85892 were selected from the ORFs having the nucleotide sequence represented by SEQ ID NO: 75617.

The probes having the nucleotide sequence represented by SEQ ID NOS: 85893, 85894, 85895, 85896, 85897, 85898, 85899, 85900, 85901, 85902, 85903 were selected from the ORFs having the nucleotide sequence represented by SEQ ID NO: 75618.

The probes having the nucleotide sequence represented by SEQ ID NOS: 85904, 85905, 85906, 85907, 85908, 85909, 85910, 85911, 85912, 85913, 85914 were selected from the ORFs having the nucleotide sequence represented by SEQ ID NO: 75619.

The probes having the nucleotide sequence represented by SEQ ID NOS: 85915, 85916, 85917, 85918, 85919, 85920, 85921, 85922, 85923, 85924, 85925 were selected from the ORFs having the nucleotide sequence represented by SEQ ID NO: 75620.

The probes having the nucleotide sequence represented by SEQ ID NOS: 85926, 85927, 85928, 85929, 85930, 85931, 85932, 85933, 85934, 85935, 85936 were selected from the ORFs having the nucleotide sequence represented by SEQ ID NO: 75621.

The probes having the nucleotide sequence represented by SEQ ID NOS: 85937, 85938, 85939, 85940, 85941, 85942, 85943, 85944, 85945, 85946, 85947 were selected from the ORFs having the nucleotide sequence represented by SEQ ID NO: 75622.

The probes having the nucleotide sequence represented by SEQ ID NOS: 85948, 85949, 85950, 85951, 85952, 85953, 85954, 85955, 85956, 85957, 85958 were selected from the ORFs having the nucleotide sequence represented by SEQ ID NO: 75623.

The probes having the nucleotide sequence represented by SEQ ID NOS: 85959, 85960, 85961, 85962, 85963, 85964, 85965, 85966, 85967, 85968, 85969 were selected from the ORFs having the nucleotide sequence represented by SEQ ID NO: 75624.

The probes having the nucleotide sequence represented by SEQ ID NOS: 85970, 85971, 85972, 85973, 85974, 85975, 85976, 85977, 85978, 85979, 85980 were selected from the ORFs having the nucleotide sequence represented by SEQ ID NO: 75625.

The probes having the nucleotide sequence represented by SEQ ID NOS: 85981, 85982, 85983, 85984, 85985, 85986, 85987, 85988, 85989, 85990, 85991 were selected from the ORFs having the nucleotide sequence represented by SEQ ID NO: 75626.

The probes having the nucleotide sequence represented by SEQ ID NOS: 85992, 85993, 85994, 85995, 85996, 85997, 85998, 85999, 86000, 86001, 86002 were selected from the ORFs having the nucleotide sequence represented by SEQ ID NO: 75627.

The probes having the nucleotide sequence represented by SEQ ID NOS: 86003, 86004, 86005, 86006, 86007, 86008, 86009, 86010, 86011, 86012, 86013 were selected from the ORFs having the nucleotide sequence represented by SEQ ID NO: 75628.

The probes having the nucleotide sequence represented by SEQ ID NOS: 86014, 86015, 86016, 86017, 86018, 86019, 86020, 86021, 86022, 86023, 86024 were selected from the ORFs having the nucleotide sequence represented by SEQ ID NO: 75629.

The probes having the nucleotide sequence represented by SEQ ID NOS: 86025, 86026, 86027, 86028, 86029, 86030, 86031, 86032, 86033, 86034, 86035 were selected from the ORFs having the nucleotide sequence represented by SEQ ID NO: 75630.

The probes having the nucleotide sequence represented by SEQ ID NOS: 86036, 86037, 86038, 86039, 86040, 86041, 86042, 86043, 86044, 86045, 86046 were selected from the ORFs having the nucleotide sequence represented by SEQ ID NO: 75631.

The probes having the nucleotide sequence represented by SEQ ID NOS: 86047, 86048, 86049, 86050, 86051, 86052, 86053, 86054, 86055, 86056, 86057 were selected from the ORFs having the nucleotide sequence represented by SEQ ID NO: 75632.

The probes having the nucleotide sequence represented by SEQ ID NOS: 86058, 86059, 86060, 86061, 86062, 86063, 86064, 86065, 86066, 86067, 86068 were selected from the ORFs having the nucleotide sequence represented by SEQ ID NO: 75633.

The probes having the nucleotide sequence represented by SEQ ID NOS: 86069, 86070, 86071, 86072, 86073, 86074, 86075, 86076, 86077, 86078, 86079 were selected from the ORFs having the nucleotide sequence represented by SEQ ID NO: 75634.

The probes having the nucleotide sequence represented by SEQ ID NOS: 86080, 86081, 86082, 86083, 86084, 86085, 86086, 86087, 86088, 86089, 86090 were selected from the ORFs having the nucleotide sequence represented by SEQ ID NO: 75635.

The probes having the nucleotide sequence represented by SEQ ID NOS: 86091, 86092, 86093, 86094, 86095, 86096, 86097, 86098, 86099, 86100, 86101 were selected from the ORFs having the nucleotide sequence represented by SEQ ID NO: 75636.

The probes having the nucleotide sequence represented by SEQ ID NOS: 86102, 86103, 86104, 86105, 86106, 86107, 86108, 86109, 86110, 86111, 86112 were selected from the ORFs having the nucleotide sequence represented by SEQ ID NO: 75637.

The probes having the nucleotide sequence represented by SEQ ID NOS: 86113, 86114, 86115, 86116, 86117, 86118, 86119, 86120, 86121, 86122, 86123 were selected from the ORFs having the nucleotide sequence represented by SEQ ID NO: 75638.

The probes having the nucleotide sequence represented by SEQ ID NOS: 86124, 86125, 86126, 86127, 86128, 86129, 86130, 86131, 86132, 86133, 86134 were selected from the ORFs having the nucleotide sequence represented by SEQ ID NO: 75639.

The probes having the nucleotide sequence represented by SEQ ID NOS: 86135, 86136, 86137, 86138, 86139, 86140, 86141, 86142, 86143, 86144, 86145 were selected from the ORFs having the nucleotide sequence represented by SEQ ID NO: 75640.

The probes having the nucleotide sequence represented by SEQ ID NOS: 86146, 86147, 86148, 86149, 86150, 86151, 86152, 86153, 86154, 86155, 86156 were selected from the ORFs having the nucleotide sequence represented by SEQ ID NO: 75641.

The probes having the nucleotide sequence represented by SEQ ID NOS: 86157, 86158, 86159, 86160, 86161, 86162, 86163, 86164, 86165, 86166, 86167 were selected from the ORFs having the nucleotide sequence represented by SEQ ID NO: 75642.

The probes having the nucleotide sequence represented by SEQ ID NOS: 86168, 86169, 86170, 86171, 86172, 86173, 86174, 86175, 86176, 86177, 86178 were selected from the ORFs having the nucleotide sequence represented by SEQ ID NO: 75643.

The probes having the nucleotide sequence represented by SEQ ID NOS: 86179, 86180, 86181, 86182, 86183, 86184, 86185, 86186, 86187, 86188, 86189 were selected from the ORFs having the nucleotide sequence represented by SEQ ID NO: 75644.

The probes having the nucleotide sequence represented by SEQ ID NOS: 86190, 86191, 86192, 86193, 86194, 86195, 86196, 86197, 86198, 86199, 86200 were selected from the ORFs having the nucleotide sequence represented by SEQ ID NO: 75645.

The probes having the nucleotide sequence represented by SEQ ID NOS: 86201, 86202, 86203, 86204, 86205, 86206, 86207, 86208, 86209, 86210, 86211 were selected from the ORFs having the nucleotide sequence represented by SEQ ID NO: 75646.

The probes having the nucleotide sequence represented by SEQ ID NOS: 86212, 86213, 86214, 86215, 86216, 86217, 86218, 86219, 86220, 86221, 86222 were selected from the ORFs having the nucleotide sequence represented by SEQ ID NO: 75647.

The probes having the nucleotide sequence represented by SEQ ID NOS: 86223, 86224, 86225, 86226, 86227, 86228, 86229, 86230, 86231, 86232, 86233 were selected from the ORFs having the nucleotide sequence represented by SEQ ID NO: 75648.

The probes having the nucleotide sequence represented by SEQ ID NOS: 86234, 86235, 86236, 86237, 86238, 86239, 86240, 86241, 86242, 86243, 86244 were selected from the ORFs having the nucleotide sequence represented by SEQ ID NO: 75649.

The probes having the nucleotide sequence represented by SEQ ID NOS: 86245, 86246, 86247, 86248, 86249, 86250, 86251, 86252, 86253, 86254, 86255 were selected from the ORFs having the nucleotide sequence represented by SEQ ID NO: 75650.

The probes having the nucleotide sequence represented by SEQ ID NOS: 86256, 86257, 86258, 86259, 86260, 86261, 86262, 86263, 86264, 86265, 86266 were selected from the ORFs having the nucleotide sequence represented by SEQ ID NO: 75651.

The probes having the nucleotide sequence represented by SEQ ID NOS: 86267, 86268, 86269, 86270, 86271, 86272, 86273, 86274, 86275, 86276, 86277 were selected from the ORFs having the nucleotide sequence represented by SEQ ID NO: 75652.

The probes having the nucleotide sequence represented by SEQ ID NOS: 86278, 86279, 86280, 86281, 86282, 86283, 86284, 86285, 86286, 86287, 86288 were selected from the ORFs having the nucleotide sequence represented by SEQ ID NO: 75653.

The probes having the nucleotide sequence represented by SEQ ID NOS: 86289, 86290, 86291, 86292, 86293, 86294, 86295, 86296, 86297, 86298, 86299 were selected from the ORFs having the nucleotide sequence represented by SEQ ID NO: 75654.

The probes having the nucleotide sequence represented by SEQ ID NOS: 86300, 86301, 86302, 86303, 86304, 86305, 86306, 86307, 86308, 86309, 86310 were selected from the ORFs having the nucleotide sequence represented by SEQ ID NO: 75655.

The probes having the nucleotide sequence represented by SEQ ID NOS: 86311, 86312, 86313, 86314, 86315, 86316, 86317, 86318, 86319, 86320, 86321 were selected from the ORFs having the nucleotide sequence represented by SEQ ID NO: 75656.

The probes having the nucleotide sequence represented by SEQ ID NOS: 86322, 86323, 86324, 86325, 86326, 86327, 86328, 86329, 86330, 86331, 86332 were selected from the ORFs having the nucleotide sequence represented by SEQ ID NO: 75657.

The probes having the nucleotide sequence represented by SEQ ID NOS: 86333, 86334, 86335, 86336, 86337, 86338, 86339, 86340, 86341, 86342, 86343 were selected from the ORFs having the nucleotide sequence represented by SEQ ID NO: 75658.

The probes having the nucleotide sequence represented by SEQ ID NOS: 86344, 86345, 86346, 86347, 86348, 86349, 86350, 86351, 86352, 86353, 86354 were selected from the ORFs having the nucleotide sequence represented by SEQ ID NO: 75659.

The probes having the nucleotide sequence represented by SEQ ID NOS: 86355, 86356, 86357, 86358, 86359, 86360, 86361, 86362, 86363, 86364, 86365 were selected from the ORFs having the nucleotide sequence represented by SEQ ID NO: 75660.

The probes having the nucleotide sequence represented by SEQ ID NOS: 86366, 86367, 86368, 86369, 86370, 86371, 86372, 86373, 86374, 86375, 86376 were selected from the ORFs having the nucleotide sequence represented by SEQ ID NO: 75661.

The probes having the nucleotide sequence represented by SEQ ID NOS: 86377, 86378, 86379, 86380, 86381, 86382, 86383, 86384, 86385, 86386, 86387 were selected from the ORFs having the nucleotide sequence represented by SEQ ID NO: 75662.

The probes having the nucleotide sequence represented by SEQ ID NOS: 86388, 86389, 86390, 86391, 86392, 86393, 86394, 86395, 86396, 86397, 86398 were selected from the ORFs having the nucleotide sequence represented by SEQ ID NO: 75663.

The probes having the nucleotide sequence represented by SEQ ID NOS: 86399, 86400, 86401, 86402, 86403, 86404, 86405, 86406, 86407, 86408, 86409 were selected from the ORFs having the nucleotide sequence represented by SEQ ID NO: 75664.

The probes having the nucleotide sequence represented by SEQ ID NOS: 86410, 86411, 86412, 86413, 86414, 86415, 86416, 86417, 86418, 86419, 86420 were selected from the ORFs having the nucleotide sequence represented by SEQ ID NO: 75665.

The probes having the nucleotide sequence represented by SEQ ID NOS: 86421, 86422, 86423, 86424, 86425, 86426, 86427, 86428, 86429, 86430, 86431 were selected from the ORFs having the nucleotide sequence represented by SEQ ID NO: 75666.

The probes having the nucleotide sequence represented by SEQ ID NOS: 86432, 86433, 86434, 86435, 86436, 86437, 86438, 86439, 86440, 86441, 86442 were selected from the ORFs having the nucleotide sequence represented by SEQ ID NO: 75667.

The probes having the nucleotide sequence represented by SEQ ID NOS: 86443, 86444, 86445, 86446, 86447, 86448, 86449, 86450, 86451, 86452, 86453 were selected from the ORFs having the nucleotide sequence represented by SEQ ID NO: 75668.

The probes having the nucleotide sequence represented by SEQ ID NOS: 86454, 86455, 86456, 86457, 86458, 86459, 86460, 86461, 86462, 86463, 86464 were selected from the ORFs having the nucleotide sequence represented by SEQ ID NO: 75669.

The probes having the nucleotide sequence represented by SEQ ID NOS: 86465, 86466, 86467, 86468, 86469, 86470, 86471, 86472, 86473, 86474, 86475 were selected from the ORFs having the nucleotide sequence represented by SEQ ID NO: 75670.

The probes having the nucleotide sequence represented by SEQ ID NOS: 86476, 86477, 86478, 86479, 86480, 86481, 86482, 86483, 86484, 86485, 86486 were selected from the ORFs having the nucleotide sequence represented by SEQ ID NO: 75671.

The probes having the nucleotide sequence represented by SEQ ID NOS: 86487, 86488, 86489, 86490, 86491, 86492, 86493, 86494, 86495, 86496, 86497 were selected from the ORFs having the nucleotide sequence represented by SEQ ID NO: 75672.

The probes having the nucleotide sequence represented by SEQ ID NOS: 86498, 86499, 86500, 86501, 86502, 86503, 86504, 86505, 86506, 86507, 86508 were selected from the ORFs having the nucleotide sequence represented by SEQ ID NO: 75673.

The probes having the nucleotide sequence represented by SEQ ID NOS: 86509, 86510, 86511, 86512, 86513, 86514, 86515, 86516, 86517, 86518, 86519 were selected from the ORFs having the nucleotide sequence represented by SEQ ID NO: 75674.

The probes having the nucleotide sequence represented by SEQ ID NOS: 86520, 86521, 86522, 86523, 86524, 86525, 86526, 86527, 86528, 86529, 86530 were selected from the ORFs having the nucleotide sequence represented by SEQ ID NO: 75675.

The probes having the nucleotide sequence represented by SEQ ID NOS: 86531, 86532, 86533, 86534, 86535, 86536, 86537, 86538, 86539, 86540, 86541 were selected from the ORFs having the nucleotide sequence represented by SEQ ID NO: 75676.

The probes having the nucleotide sequence represented by SEQ ID NOS: 86542, 86543, 86544, 86545, 86546, 86547, 86548, 86549, 86550, 86551, 86552 were selected from the ORFs having the nucleotide sequence represented by SEQ ID NO: 75677.

The probes having the nucleotide sequence represented by SEQ ID NOS: 86553, 86554, 86555, 86556, 86557, 86558, 86559, 86560, 86561, 86562, 86563 were selected from the ORFs having the nucleotide sequence represented by SEQ ID NO: 75678.

The probes having the nucleotide sequence represented by SEQ ID NOS: 86564, 86565, 86566, 86567, 86568, 86569, 86570, 86571, 86572, 86573, 86574 were selected from the ORFs having the nucleotide sequence represented by SEQ ID NO: 75679.

The probes having the nucleotide sequence represented by SEQ ID NOS: 86575, 86576, 86577, 86578, 86579, 86580, 86581, 86582, 86583, 86584, 86585 were selected from the ORFs having the nucleotide sequence represented by SEQ ID NO: 75680.

The probes having the nucleotide sequence represented by SEQ ID NOS: 86586, 86587, 86588, 86589, 86590, 86591, 86592, 86593, 86594, 86595, 86596 were selected from the ORFs having the nucleotide sequence represented by SEQ ID NO: 75681.

The probes having the nucleotide sequence represented by SEQ ID NOS: 86597, 86598, 86599, 86600, 86601, 86602, 86603, 86604, 86605, 86606, 86607 were selected from the ORFs having the nucleotide sequence represented by SEQ ID NO: 75682.

The probes having the nucleotide sequence represented by SEQ ID NOS: 86608, 86609, 86610, 86611, 86612, 86613, 86614, 86615, 86616, 86617, 86618 were selected from the ORFs having the nucleotide sequence represented by SEQ ID NO: 75683.

The probes having the nucleotide sequence represented by SEQ ID NOS: 86619, 86620, 86621, 86622, 86623, 86624, 86625, 86626, 86627, 86628, 86629 were selected from the ORFs having the nucleotide sequence represented by SEQ ID NO: 75684.

The probes having the nucleotide sequence represented by SEQ ID NOS: 86630, 86631, 86632, 86633, 86634, 86635, 86636, 86637, 86638, 86639, 86640 were selected from the ORFs having the nucleotide sequence represented by SEQ ID NO: 75685.

The probes having the nucleotide sequence represented by SEQ ID NOS: 86641, 86642, 86643, 86644, 86645, 86646, 86647, 86648, 86649, 86650, 86651 were selected from the ORFs having the nucleotide sequence represented by SEQ ID NO: 75686.

The probes having the nucleotide sequence represented by SEQ ID NOS: 86652, 86653, 86654, 86655, 86656, 86657, 86658, 86659, 86660, 86661, 86662 were selected from the ORFs having the nucleotide sequence represented by SEQ ID NO: 75687.

The probes having the nucleotide sequence represented by SEQ ID NOS: 86663, 86664, 86665, 86666, 86667, 86668, 86669, 86670, 86671, 86672, 86673 were selected from the ORFs having the nucleotide sequence represented by SEQ ID NO: 75688.

The probes having the nucleotide sequence represented by SEQ ID NOS: 86674, 86675, 86676, 86677, 86678, 86679, 86680, 86681, 86682, 86683, 86684 were selected from the ORFs having the nucleotide sequence represented by SEQ ID NO: 75689.

The probes having the nucleotide sequence represented by SEQ ID NOS: 86685, 86686, 86687, 86688, 86689, 86690, 86691, 86692, 86693, 86694, 86695 were selected from the ORFs having the nucleotide sequence represented by SEQ ID NO: 75690.

The probes having the nucleotide sequence represented by SEQ ID NOS: 86696, 86697, 86698, 86699, 86700, 86701, 86702, 86703, 86704, 86705, 86706 were selected from the ORFs having the nucleotide sequence represented by SEQ ID NO: 75691.

The probes having the nucleotide sequence represented by SEQ ID NOS: 86707, 86708, 86709, 86710, 86711, 86712, 86713, 86714, 86715, 86716, 86717 were selected from the ORFs having the nucleotide sequence represented by SEQ ID NO: 75692.

The probes having the nucleotide sequence represented by SEQ ID NOS: 86718, 86719, 86720, 86721, 86722, 86723, 86724, 86725, 86726, 86727, 86728 were selected from the ORFs having the nucleotide sequence represented by SEQ ID NO: 75693.

The probes having the nucleotide sequence represented by SEQ ID NOS: 86729, 86730, 86731, 86732, 86733, 86734, 86735, 86736, 86737, 86738, 86739 were selected from the ORFs having the nucleotide sequence represented by SEQ ID NO: 75694.

The probes having the nucleotide sequence represented by SEQ ID NOS: 86740, 86741, 86742, 86743, 86744, 86745, 86746, 86747, 86748, 86749, 86750 were selected from the ORFs having the nucleotide sequence represented by SEQ ID NO: 75695.

The probes having the nucleotide sequence represented by SEQ ID NOS: 86751, 86752, 86753, 86754, 86755, 86756, 86757, 86758, 86759, 86760, 86761 were selected from the ORFs having the nucleotide sequence represented by SEQ ID NO: 75696.

The probes having the nucleotide sequence represented by SEQ ID NOS: 86762, 86763, 86764, 86765, 86766, 86767, 86768, 86769, 86770, 86771, 86772 were selected from the ORFs having the nucleotide sequence represented by SEQ ID NO: 75697.

The probes having the nucleotide sequence represented by SEQ ID NOS: 86773, 86774, 86775, 86776, 86777, 86778, 86779, 86780, 86781, 86782, 86783 were selected from the ORFs having the nucleotide sequence represented by SEQ ID NO: 75698.

The probes having the nucleotide sequence represented by SEQ ID NOS: 86784, 86785, 86786, 86787, 86788, 86789, 86790, 86791, 86792, 86793, 86794 were selected from the ORFs having the nucleotide sequence represented by SEQ ID NO: 75699.

The probes having the nucleotide sequence represented by SEQ ID NOS: 86795, 86796, 86797, 86798, 86799, 86800, 86801, 86802, 86803, 86804, 86805 were selected from the ORFs having the nucleotide sequence represented by SEQ ID NO: 75700.

The probes having the nucleotide sequence represented by SEQ ID NOS: 86806, 86807, 86808, 86809, 86810, 86811, 86812, 86813, 86814, 86815, 86816 were selected from the ORFs having the nucleotide sequence represented by SEQ ID NO: 75701.

The probes having the nucleotide sequence represented by SEQ ID NOS: 86817, 86818, 86819, 86820, 86821, 86822, 86823, 86824, 86825, 86826, 86827 were selected from the ORFs having the nucleotide sequence represented by SEQ ID NO: 75702.

The probes having the nucleotide sequence represented by SEQ ID NOS: 86828, 86829, 86830, 86831, 86832, 86833, 86834, 86835, 86836, 86837, 86838 were selected from the ORFs having the nucleotide sequence represented by SEQ ID NO: 75703.

The probes having the nucleotide sequence represented by SEQ ID NOS: 86839, 86840, 86841, 86842, 86843, 86844, 86845, 86846, 86847, 86848, 86849 were selected from the ORFs having the nucleotide sequence represented by SEQ ID NO: 75704.

The probes having the nucleotide sequence represented by SEQ ID NOS: 86850, 86851, 86852, 86853, 86854, 86855, 86856, 86857, 86858, 86859, 86860 were selected from the ORFs having the nucleotide sequence represented by SEQ ID NO: 75705.

The probes having the nucleotide sequence represented by SEQ ID NOS: 86861, 86862, 86863, 86864, 86865, 86866, 86867, 86868, 86869, 86870, 86871 were selected from the ORFs having the nucleotide sequence represented by SEQ ID NO: 75706.

The probes having the nucleotide sequence represented by SEQ ID NOS: 86872, 86873, 86874, 86875, 86876, 86877, 86878, 86879, 86880, 86881, 86882 were selected from the ORFs having the nucleotide sequence represented by SEQ ID NO: 75707.

The probes having the nucleotide sequence represented by SEQ ID NOS: 86883, 86884, 86885, 86886, 86887, 86888, 86889, 86890, 86891, 86892, 86893 were selected from the ORFs having the nucleotide sequence represented by SEQ ID NO: 75708.

The probes having the nucleotide sequence represented by SEQ ID NOS: 86894, 86895, 86896, 86897, 86898, 86899, 86900, 86901, 86902, 86903, 86904 were selected from the ORFs having the nucleotide sequence represented by SEQ ID NO: 75709.

The probes having the nucleotide sequence represented by SEQ ID NOS: 86905, 86906, 86907, 86908, 86909, 86910, 86911, 86912, 86913, 86914, 86915 were selected from the ORFs having the nucleotide sequence represented by SEQ ID NO: 75710.

The probes having the nucleotide sequence represented by SEQ ID NOS: 86916, 86917, 86918, 86919, 86920, 86921, 86922, 86923, 86924, 86925, 86926 were selected from the ORFs having the nucleotide sequence represented by SEQ ID NO: 75711.

The probes having the nucleotide sequence represented by SEQ ID NOS: 86927, 86928, 86929, 86930, 86931, 86932, 86933, 86934, 86935, 86936, 86937 were selected from the ORFs having the nucleotide sequence represented by SEQ ID NO: 75712.

The probes having the nucleotide sequence represented by SEQ ID NOS: 86938, 86939, 86940, 86941, 86942, 86943, 86944, 86945, 86946, 86947, 86948 were selected from the ORFs having the nucleotide sequence represented by SEQ ID NO: 75713.

The probes having the nucleotide sequence represented by SEQ ID NOS: 86949, 86950, 86951, 86952, 86953, 86954, 86955, 86956, 86957, 86958, 86959 were selected from the ORFs having the nucleotide sequence represented by SEQ ID NO: 75714.

The probes having the nucleotide sequence represented by SEQ ID NOS: 86960, 86961, 86962, 86963, 86964, 86965, 86966, 86967, 86968, 86969, 86970 were selected from the ORFs having the nucleotide sequence represented by SEQ ID NO: 75715.

The probes having the nucleotide sequence represented by SEQ ID NOS: 86971, 86972, 86973, 86974, 86975, 86976, 86977, 86978, 86979, 86980, 86981 were selected from the ORFs having the nucleotide sequence represented by SEQ ID NO: 75716.

The probes having the nucleotide sequence represented by SEQ ID NOS: 86982, 86983, 86984, 86985, 86986, 86987, 86988, 86989, 86990, 86991, 86992 were selected from the ORFs having the nucleotide sequence represented by SEQ ID NO: 75717.

The probes having the nucleotide sequence represented by SEQ ID NOS: 86993, 86994, 86995, 86996, 86997, 86998, 86999, 87000, 87001, 87002, 87003 were selected from the ORFs having the nucleotide sequence represented by SEQ ID NO: 75718.

The probes having the nucleotide sequence represented by SEQ ID NOS: 87004, 87005, 87006, 87007, 87008, 87009, 87010, 87011, 87012, 87013, 87014 were selected from the ORFs having the nucleotide sequence represented by SEQ ID NO: 75719.

The probes having the nucleotide sequence represented by SEQ ID NOS: 87015, 87016, 87017, 87018, 87019, 87020, 87021, 87022, 87023, 87024, 87025 were selected from the ORFs having the nucleotide sequence represented by SEQ ID NO: 75720.

The probes having the nucleotide sequence represented by SEQ ID NOS: 87026, 87027, 87028, 87029, 87030, 87031, 87032, 87033, 87034, 87035, 87036 were selected from the ORFs having the nucleotide sequence represented by SEQ ID NO: 75721.

The probes having the nucleotide sequence represented by SEQ ID NOS: 87037, 87038, 87039, 87040, 87041, 87042, 87043, 87044, 87045, 87046, 87047 were selected from the ORFs having the nucleotide sequence represented by SEQ ID NO: 75722.

The probes having the nucleotide sequence represented by SEQ ID NOS: 87048, 87049, 87050, 87051, 87052, 87053, 87054, 87055, 87056, 87057, 87058 were selected from the ORFs having the nucleotide sequence represented by SEQ ID NO: 75723.

The probes having the nucleotide sequence represented by SEQ ID NOS: 87059, 87060, 87061, 87062, 87063, 87064, 87065, 87066, 87067, 87068, 87069 were selected from the ORFs having the nucleotide sequence represented by SEQ ID NO: 75724.

The probes having the nucleotide sequence represented by SEQ ID NOS: 87070, 87071, 87072, 87073, 87074, 87075, 87076, 87077, 87078, 87079, 87080 were selected from the ORFs having the nucleotide sequence represented by SEQ ID NO: 75725.

The probes having the nucleotide sequence represented by SEQ ID NOS: 87081, 87082, 87083, 87084, 87085, 87086, 87087, 87088, 87089, 87090, 87091 were selected from the ORFs having the nucleotide sequence represented by SEQ ID NO: 75726.

The probes having the nucleotide sequence represented by SEQ ID NOS: 87092, 87093, 87094, 87095, 87096, 87097, 87098, 87099, 87100, 87101, 87102 were selected from the ORFs having the nucleotide sequence represented by SEQ ID NO: 75727.

The probes having the nucleotide sequence represented by SEQ ID NOS: 87103, 87104, 87105, 87106, 87107, 87108, 87109, 87110, 87111, 87112, 87113 were selected from the ORFs having the nucleotide sequence represented by SEQ ID NO: 75728.

The probes having the nucleotide sequence represented by SEQ ID NOS: 87114, 87115, 87116, 87117, 87118, 87119, 87120, 87121, 87122, 87123, 87124 were selected from the ORFs having the nucleotide sequence represented by SEQ ID NO: 75729.

The probes having the nucleotide sequence represented by SEQ ID NOS: 87125, 87126, 87127, 87128, 87129, 87130, 87131, 87132, 87133, 87134, 87135 were selected from the ORFs having the nucleotide sequence represented by SEQ ID NO: 75730.

The probes having the nucleotide sequence represented by SEQ ID NOS: 87136, 87137, 87138, 87139, 87140, 87141, 87142, 87143, 87144, 87145, 87146 were selected from the ORFs having the nucleotide sequence represented by SEQ ID NO: 75731.

The probes having the nucleotide sequence represented by SEQ ID NOS: 87147, 87148, 87149, 87150, 87151, 87152, 87153, 87154, 87155, 87156, 87157 were selected from the ORFs having the nucleotide sequence represented by SEQ ID NO: 75732.

The probes having the nucleotide sequence represented by SEQ ID NOS: 87158, 87159, 87160, 87161, 87162, 87163, 87164, 87165, 87166, 87167, 87168 were selected from the ORFs having the nucleotide sequence represented by SEQ ID NO: 75733.

The probes having the nucleotide sequence represented by SEQ ID NOS: 87169, 87170, 87171, 87172, 87173, 87174, 87175, 87176, 87177, 87178, 87179 were selected from the ORFs having the nucleotide sequence represented by SEQ ID NO: 75734.

The probes having the nucleotide sequence represented by SEQ ID NOS: 87180, 87181, 87182, 87183, 87184, 87185, 87186, 87187, 87188, 87189, 87190 were selected from the ORFs having the nucleotide sequence represented by SEQ ID NO: 75735.

The probes having the nucleotide sequence represented by SEQ ID NOS: 87191, 87192, 87193, 87194, 87195, 87196, 87197, 87198, 87199, 87200, 87201 were selected from the ORFs having the nucleotide sequence represented by SEQ ID NO: 75736.

The probes having the nucleotide sequence represented by SEQ ID NOS: 87202, 87203, 87204, 87205, 87206, 87207, 87208, 87209, 87210, 87211, 87212 were selected from the ORFs having the nucleotide sequence represented by SEQ ID NO: 75737.

The probes having the nucleotide sequence represented by SEQ ID NOS: 87213, 87214, 87215, 87216, 87217, 87218, 87219, 87220, 87221, 87222, 87223 were selected from the ORFs having the nucleotide sequence represented by SEQ ID NO: 75738.

The probes having the nucleotide sequence represented by SEQ ID NOS: 87224, 87225, 87226, 87227, 87228, 87229, 87230, 87231, 87232, 87233, 87234 were selected from the ORFs having the nucleotide sequence represented by SEQ ID NO: 75739.

The probes having the nucleotide sequence represented by SEQ ID NOS: 87235, 87236, 87237, 87238, 87239, 87240, 87241, 87242, 87243, 87244, 87245 were selected from the ORFs having the nucleotide sequence represented by SEQ ID NO: 75740.

The probes having the nucleotide sequence represented by SEQ ID NOS: 87246, 87247, 87248, 87249, 87250, 87251, 87252, 87253, 87254, 87255, 87256 were selected from the ORFs having the nucleotide sequence represented by SEQ ID NO: 75741.

The probes having the nucleotide sequence represented by SEQ ID NOS: 87257, 87258, 87259, 87260, 87261, 87262, 87263, 87264, 87265, 87266, 87267 were selected from the ORFs having the nucleotide sequence represented by SEQ ID NO: 75742.

The probes having the nucleotide sequence represented by SEQ ID NOS: 87268, 87269, 87270, 87271, 87272, 87273, 87274, 87275, 87276, 87277, 87278 were selected from the ORFs having the nucleotide sequence represented by SEQ ID NO: 75743.

The probes having the nucleotide sequence represented by SEQ ID NOS: 87279, 87280, 87281, 87282, 87283, 87284, 87285, 87286, 87287, 87288, 87289 were selected from the ORFs having the nucleotide sequence represented by SEQ ID NO: 75744.

The probes having the nucleotide sequence represented by SEQ ID NOS: 87290, 87291, 87292, 87293, 87294, 87295, 87296, 87297, 87298, 87299, 87300 were selected from the ORFs having the nucleotide sequence represented by SEQ ID NO: 75745.

The probes having the nucleotide sequence represented by SEQ ID NOS: 87301, 87302, 87303, 87304, 87305, 87306, 87307, 87308, 87309, 87310, 87311 were selected from the ORFs having the nucleotide sequence represented by SEQ ID NO: 75746.

The probes having the nucleotide sequence represented by SEQ ID NOS: 87312, 87313, 87314, 87315, 87316, 87317, 87318, 87319, 87320, 87321, 87322 were selected from the ORFs having the nucleotide sequence represented by SEQ ID NO: 75747.

The probes having the nucleotide sequence represented by SEQ ID NOS: 87323, 87324, 87325, 87326, 87327, 87328, 87329, 87330, 87331, 87332, 87333 were selected from the ORFs having the nucleotide sequence represented by SEQ ID NO: 75748.

The probes having the nucleotide sequence represented by SEQ ID NOS: 87334, 87335, 87336, 87337, 87338, 87339, 87340, 87341, 87342, 87343, 87344 were selected from the ORFs having the nucleotide sequence represented by SEQ ID NO: 75749.

The probes having the nucleotide sequence represented by SEQ ID NOS: 87345, 87346, 87347, 87348, 87349, 87350, 87351, 87352, 87353, 87354, 87355 were selected from the ORFs having the nucleotide sequence represented by SEQ ID NO: 75750.

The probes having the nucleotide sequence represented by SEQ ID NOS: 87356, 87357, 87358, 87359, 87360, 87361, 87362, 87363, 87364, 87365, 87366 were selected from the ORFs having the nucleotide sequence represented by SEQ ID NO: 75751.

The probes having the nucleotide sequence represented by SEQ ID NOS: 87367, 87368, 87369, 87370, 87371, 87372, 87373, 87374, 87375, 87376, 87377 were selected from the ORFs having the nucleotide sequence represented by SEQ ID NO: 75752.

The probes having the nucleotide sequence represented by SEQ ID NOS: 87378, 87379, 87380, 87381, 87382, 87383, 87384, 87385, 87386, 87387, 87388 were selected from the ORFs having the nucleotide sequence represented by SEQ ID NO: 75753.

The probes having the nucleotide sequence represented by SEQ ID NOS: 87389, 87390, 87391, 87392, 87393, 87394, 87395, 87396, 87397, 87398, 87399 were selected from the ORFs having the nucleotide sequence represented by SEQ ID NO: 75754.

The probes having the nucleotide sequence represented by SEQ ID NOS: 87400, 87401, 87402, 87403, 87404, 87405, 87406, 87407, 87408, 87409, 87410 were selected from the ORFs having the nucleotide sequence represented by SEQ ID NO: 75755.

The probes having the nucleotide sequence represented by SEQ ID NOS: 87411, 87412, 87413, 87414, 87415, 87416, 87417, 87418, 87419, 87420, 87421 were selected from the ORFs having the nucleotide sequence represented by SEQ ID NO: 75756.

The probes having the nucleotide sequence represented by SEQ ID NOS: 87422, 87423, 87424, 87425, 87426, 87427, 87428, 87429, 87430, 87431, 87432 were selected from the ORFs having the nucleotide sequence represented by SEQ ID NO: 75757.

The probes having the nucleotide sequence represented by SEQ ID NOS: 87433, 87434, 87435, 87436, 87437, 87438, 87439, 87440, 87441, 87442, 87443 were selected from the ORFs having the nucleotide sequence represented by SEQ ID NO: 75758.

The probes having the nucleotide sequence represented by SEQ ID NOS: 87444, 87445, 87446, 87447, 87448, 87449, 87450, 87451, 87452, 87453, 87454 were selected from the ORFs having the nucleotide sequence represented by SEQ ID NO: 75759.

The probes having the nucleotide sequence represented by SEQ ID NOS: 87455, 87456, 87457, 87458, 87459, 87460, 87461, 87462, 87463, 87464, 87465 were selected from the ORFs having the nucleotide sequence represented by SEQ ID NO: 75760.

The probes having the nucleotide sequence represented by SEQ ID NOS: 87466, 87467, 87468, 87469, 87470, 87471, 87472, 87473, 87474, 87475, 87476 were selected from the ORFs having the nucleotide sequence represented by SEQ ID NO: 75761.

The probes having the nucleotide sequence represented by SEQ ID NOS: 87477, 87478, 87479, 87480, 87481, 87482, 87483, 87484, 87485, 87486, 87487 were selected from the ORFs having the nucleotide sequence represented by SEQ ID NO: 75762.

The probes having the nucleotide sequence represented by SEQ ID NOS: 87488, 87489, 87490, 87491, 87492, 87493, 87494, 87495, 87496, 87497, 87498 were selected from the ORFs having the nucleotide sequence represented by SEQ ID NO: 75763.

The probes having the nucleotide sequence represented by SEQ ID NOS: 87499, 87500, 87501, 87502, 87503, 87504, 87505, 87506, 87507, 87508, 87509 were selected from the ORFs having the nucleotide sequence represented by SEQ ID NO: 75764.

The probes having the nucleotide sequence represented by SEQ ID NOS: 87510, 87511, 87512, 87513, 87514, 87515, 87516, 87517, 87518, 87519, 87520 were selected from the ORFs having the nucleotide sequence represented by SEQ ID NO: 75765.

The probes having the nucleotide sequence represented by SEQ ID NOS: 87521, 87522, 87523, 87524, 87525, 87526, 87527, 87528, 87529, 87530, 87531 were selected from the ORFs having the nucleotide sequence represented by SEQ ID NO: 75766.

The probes having the nucleotide sequence represented by SEQ ID NOS: 87532, 87533, 87534, 87535, 87536, 87537, 87538, 87539, 87540, 87541, 87542 were selected from the ORFs having the nucleotide sequence represented by SEQ ID NO: 75767.

The probes having the nucleotide sequence represented by SEQ ID NOS: 87543, 87544, 87545, 87546, 87547, 87548, 87549, 87550, 87551, 87552, 87553 were selected from the ORFs having the nucleotide sequence represented by SEQ ID NO: 75768.

The probes having the nucleotide sequence represented by SEQ ID NOS: 87554, 87555, 87556, 87557, 87558, 87559, 87560, 87561, 87562, 87563, 87564 were selected from the ORFs having the nucleotide sequence represented by SEQ ID NO: 75769.

The probes having the nucleotide sequence represented by SEQ ID NOS: 87565, 87566, 87567, 87568, 87569, 87570, 87571, 87572, 87573, 87574, 87575 were selected from the ORFs having the nucleotide sequence represented by SEQ ID NO: 75770.

The probes having the nucleotide sequence represented by SEQ ID NOS: 87576, 87577, 87578, 87579, 87580, 87581, 87582, 87583, 87584, 87585, 87586 were selected from the ORFs having the nucleotide sequence represented by SEQ ID NO: 75771.

The probes having the nucleotide sequence represented by SEQ ID NOS: 87587, 87588, 87589, 87590, 87591, 87592, 87593, 87594, 87595, 87596, 87597 were selected from the ORFs having the nucleotide sequence represented by SEQ ID NO: 75772.

The probes having the nucleotide sequence represented by SEQ ID NOS: 87598, 87599, 87600, 87601, 87602, 87603, 87604, 87605, 87606, 87607, 87608 were selected from the ORFs having the nucleotide sequence represented by SEQ ID NO: 75773.

The probes having the nucleotide sequence represented by SEQ ID NOS: 87609, 87610, 87611, 87612, 87613, 87614, 87615, 87616, 87617, 87618, 87619 were selected from the ORFs having the nucleotide sequence represented by SEQ ID NO: 75774.

The probes having the nucleotide sequence represented by SEQ ID NOS: 87620, 87621, 87622, 87623, 87624, 87625, 87626, 87627, 87628, 87629, 87630 were selected from the ORFs having the nucleotide sequence represented by SEQ ID NO: 75775.

The probes having the nucleotide sequence represented by SEQ ID NOS: 87631, 87632, 87633, 87634, 87635, 87636, 87637, 87638, 87639, 87640, 87641 were selected from the ORFs having the nucleotide sequence represented by SEQ ID NO: 75776.

The probes having the nucleotide sequence represented by SEQ ID NOS: 87642, 87643, 87644, 87645, 87646, 87647, 87648, 87649, 87650, 87651, 87652 were selected from the ORFs having the nucleotide sequence represented by SEQ ID NO: 75777.

The probes having the nucleotide sequence represented by SEQ ID NOS: 87653, 87654, 87655, 87656, 87657, 87658, 87659, 87660, 87661, 87662, 87663 were selected from the ORFs having the nucleotide sequence represented by SEQ ID NO: 75778.

The probes having the nucleotide sequence represented by SEQ ID NOS: 87664, 87665, 87666, 87667, 87668, 87669, 87670, 87671, 87672, 87673, 87674 were selected from the ORFs having the nucleotide sequence represented by SEQ ID NO: 75779.

The probes having the nucleotide sequence represented by SEQ ID NOS: 87675, 87676, 87677, 87678, 87679, 87680, 87681, 87682, 87683, 87684, 87685 were selected from the ORFs having the nucleotide sequence represented by SEQ ID NO: 75780.

The probes having the nucleotide sequence represented by SEQ ID NOS: 87686, 87687, 87688, 87689, 87690, 87691, 87692, 87693, 87694, 87695, 87696 were selected from the ORFs having the nucleotide sequence represented by SEQ ID NO: 75781.

The probes having the nucleotide sequence represented by SEQ ID NOS: 87697, 87698, 87699, 87700, 87701, 87702, 87703, 87704, 87705, 87706, 87707 were selected from the ORFs having the nucleotide sequence represented by SEQ ID NO: 75782.

The probes having the nucleotide sequence represented by SEQ ID NOS: 87708, 87709, 87710, 87711, 87712, 87713, 87714, 87715, 87716, 87717, 87718 were selected from the ORFs having the nucleotide sequence represented by SEQ ID NO: 75783.

The probes having the nucleotide sequence represented by SEQ ID NOS: 87719, 87720, 87721, 87722, 87723, 87724, 87725, 87726, 87727, 87728, 87729 were selected from the ORFs having the nucleotide sequence represented by SEQ ID NO: 75784.

The probes having the nucleotide sequence represented by SEQ ID NOS: 87730, 87731, 87732, 87733, 87734, 87735, 87736, 87737, 87738, 87739, 87740 were selected from the ORFs having the nucleotide sequence represented by SEQ ID NO: 75785.

The probes having the nucleotide sequence represented by SEQ ID NOS: 87741, 87742, 87743, 87744, 87745, 87746, 87747, 87748, 87749, 87750, 87751 were selected from the ORFs having the nucleotide sequence represented by SEQ ID NO: 75786.

The probes having the nucleotide sequence represented by SEQ ID NOS: 87752, 87753, 87754, 87755, 87756, 87757, 87758, 87759, 87760, 87761, 87762 were selected from the ORFs having the nucleotide sequence represented by SEQ ID NO: 75787.

The probes having the nucleotide sequence represented by SEQ ID NOS: 87763, 87764, 87765, 87766, 87767, 87768, 87769, 87770, 87771, 87772, 87773 were selected from the ORFs having the nucleotide sequence represented by SEQ ID NO: 75788.

The probes having the nucleotide sequence represented by SEQ ID NOS: 87774, 87775, 87776, 87777, 87778, 87779, 87780, 87781, 87782, 87783, 87784 were selected from the ORFs having the nucleotide sequence represented by SEQ ID NO: 75789.

The probes having the nucleotide sequence represented by SEQ ID NOS: 87785, 87786, 87787, 87788, 87789, 87790, 87791, 87792, 87793, 87794, 87795 were selected from the ORFs having the nucleotide sequence represented by SEQ ID NO: 75790.

The probes having the nucleotide sequence represented by SEQ ID NOS: 87796, 87797, 87798, 87799, 87800, 87801, 87802, 87803, 87804, 87805, 87806 were selected from the ORFs having the nucleotide sequence represented by SEQ ID NO: 75791.

The probes having the nucleotide sequence represented by SEQ ID NOS: 87807, 87808, 87809, 87810, 87811, 87812, 87813, 87814, 87815, 87816, 87817 were selected from the ORFs having the nucleotide sequence represented by SEQ ID NO: 75792.

The probes having the nucleotide sequence represented by SEQ ID NOS: 87818, 87819, 87820, 87821, 87822, 87823, 87824, 87825, 87826, 87827, 87828, 87829, 87830, 87831, 87832, 87833, 87834, 87835, 87836, 87837, 87838, 87839 were selected from the ORFs having the nucleotide sequence represented by SEQ ID NO: 75793.

The probes having the nucleotide sequence represented by SEQ ID NOS: 87840, 87841, 87842, 87843, 87844, 87845, 87846, 87847, 87848, 87849, 87850 were selected from the ORFs having the nucleotide sequence represented by SEQ ID NO: 75794.

The probes having the nucleotide sequence represented by SEQ ID NOS: 87851, 87852, 87853, 87854, 87855, 87856, 87857, 87858, 87859, 87860, 87861 were selected from the ORFs having the nucleotide sequence represented by SEQ ID NO: 75795.

The probes having the nucleotide sequence represented by SEQ ID NOS: 87862, 87863, 87864, 87865, 87866, 87867, 87868, 87869, 87870, 87871, 87872 were selected from the ORFs having the nucleotide sequence represented by SEQ ID NO: 75796.

The probes having the nucleotide sequence represented by SEQ ID NOS: 87873, 87874, 87875, 87876, 87877, 87878, 87879, 87880, 87881, 87882, 87883 were selected from the ORFs having the nucleotide sequence represented by SEQ ID NO: 75797.

The probes having the nucleotide sequence represented by SEQ ID NOS: 87884, 87885, 87886, 87887, 87888, 87889, 87890, 87891, 87892, 87893, 87894 were selected from the ORFs having the nucleotide sequence represented by SEQ ID NO: 75798.

The probes having the nucleotide sequence represented by SEQ ID NOS: 87895, 87896, 87897, 87898, 87899, 87900, 87901, 87902, 87903, 87904, 87905 were selected from the ORFs having the nucleotide sequence represented by SEQ ID NO: 75799.

The probes having the nucleotide sequence represented by SEQ ID NOS: 87906, 87907, 87908, 87909, 87910, 87911, 87912, 87913, 87914, 87915, 87916 were selected from the ORFs having the nucleotide sequence represented by SEQ ID NO: 75800.

The probes having the nucleotide sequence represented by SEQ ID NOS: 87917, 87918, 87919, 87920, 87921, 87922, 87923, 87924, 87925, 87926, 87927 were selected from the ORFs having the nucleotide sequence represented by SEQ ID NO: 75801.

The probes having the nucleotide sequence represented by SEQ ID NOS: 87928, 87929, 87930, 87931, 87932, 87933, 87934, 87935, 87936, 87937, 87938 were selected from the ORFs having the nucleotide sequence represented by SEQ ID NO: 75802.

The probes having the nucleotide sequence represented by SEQ ID NOS: 87939, 87940, 87941, 87942, 87943, 87944, 87945, 87946, 87947, 87948, 87949 were selected from the ORFs having the nucleotide sequence represented by SEQ ID NO: 75803.

The probes having the nucleotide sequence represented by SEQ ID NOS: 87950, 87951, 87952, 87953, 87954, 87955, 87956, 87957, 87958, 87959, 87960 were selected from the ORFs having the nucleotide sequence represented by SEQ ID NO: 75804.

The probes having the nucleotide sequence represented by SEQ ID NOS: 87961, 87962, 87963, 87964, 87965, 87966, 87967, 87968, 87969, 87970, 87971 were selected from the ORFs having the nucleotide sequence represented by SEQ ID NO: 75805.

The probes having the nucleotide sequence represented by SEQ ID NOS: 87972, 87973, 87974, 87975, 87976, 87977, 87978, 87979, 87980, 87981, 87982 were selected from the ORFs having the nucleotide sequence represented by SEQ ID NO: 75806.

The probes having the nucleotide sequence represented by SEQ ID NOS: 87983, 87984, 87985, 87986, 87987, 87988, 87989, 87990, 87991, 87992, 87993 were selected from the ORFs having the nucleotide sequence represented by SEQ ID NO: 75807.

The probes having the nucleotide sequence represented by SEQ ID NOS: 87994, 87995, 87996, 87997, 87998, 87999, 88000, 88001, 88002, 88003, 88004 were selected from the ORFs having the nucleotide sequence represented by SEQ ID NO: 75808.

The probes having the nucleotide sequence represented by SEQ ID NOS: 88005, 88006, 88007, 88008, 88009, 88010, 88011, 88012, 88013, 88014, 88015 were selected from the ORFs having the nucleotide sequence represented by SEQ ID NO: 75809.

The probes having the nucleotide sequence represented by SEQ ID NOS: 88016, 88017, 88018, 88019, 88020, 88021, 88022, 88023, 88024, 88025, 88026 were selected from the ORFs having the nucleotide sequence represented by SEQ ID NO: 75810.

The probes having the nucleotide sequence represented by SEQ ID NOS: 88027, 88028, 88029, 88030, 88031, 88032, 88033, 88034, 88035, 88036, 88037 were selected from the ORFs having the nucleotide sequence represented by SEQ ID NO: 75811.

The probes having the nucleotide sequence represented by SEQ ID NOS: 88038, 88039, 88040, 88041, 88042, 88043, 88044, 88045, 88046, 88047, 88048 were selected from the ORFs having the nucleotide sequence represented by SEQ ID NO: 75812.

The probes having the nucleotide sequence represented by SEQ ID NOS: 88049, 88050, 88051, 88052, 88053, 88054, 88055, 88056, 88057, 88058, 88059 were selected from the ORFs having the nucleotide sequence represented by SEQ ID NO: 75813.

The probes having the nucleotide sequence represented by SEQ ID NOS: 88060, 88061, 88062, 88063, 88064, 88065, 88066, 88067, 88068, 88069, 88070 were selected from the ORFs having the nucleotide sequence represented by SEQ ID NO: 75814.

The probes having the nucleotide sequence represented by SEQ ID NOS: 88071, 88072, 88073, 88074, 88075, 88076, 88077, 88078, 88079, 88080, 88081 were selected from the ORFs having the nucleotide sequence represented by SEQ ID NO: 75815.

The probes having the nucleotide sequence represented by SEQ ID NOS: 88082, 88083, 88084, 88085, 88086, 88087, 88088, 88089, 88090, 88091, 88092 were selected from the ORFs having the nucleotide sequence represented by SEQ ID NO: 75816.

The probes having the nucleotide sequence represented by SEQ ID NOS: 88093, 88094, 88095, 88096, 88097, 88098, 88099, 88100, 88101, 88102, 88103 were selected from the ORFs having the nucleotide sequence represented by SEQ ID NO: 75817.

The probes having the nucleotide sequence represented by SEQ ID NOS: 88104, 88105, 88106, 88107, 88108, 88109, 88110, 88111, 88112, 88113, 88114 were selected from the ORFs having the nucleotide sequence represented by SEQ ID NO: 75818.

The probes having the nucleotide sequence represented by SEQ ID NOS: 88115, 88116, 88117, 88118, 88119, 88120, 88121, 88122, 88123, 88124, 88125 were selected from the ORFs having the nucleotide sequence represented by SEQ ID NO: 75819.

The probes having the nucleotide sequence represented by SEQ ID NOS: 88126, 88127, 88128, 88129, 88130, 88131, 88132, 88133, 88134, 88135, 88136 were selected from the ORFs having the nucleotide sequence represented by SEQ ID NO: 75820.

The probes having the nucleotide sequence represented by SEQ ID NOS: 88137, 88138, 88139, 88140, 88141, 88142, 88143, 88144, 88145, 88146, 88147 were selected from the ORFs having the nucleotide sequence represented by SEQ ID NO: 75821.

The probes having the nucleotide sequence represented by SEQ ID NOS: 88148, 88149, 88150, 88151, 88152, 88153, 88154, 88155, 88156, 88157, 88158 were selected from the ORFs having the nucleotide sequence represented by SEQ ID NO: 75822.

The probes having the nucleotide sequence represented by SEQ ID NOS: 88159, 88160, 88161, 88162, 88163, 88164, 88165, 88166, 88167, 88168, 88169 were selected from the ORFs having the nucleotide sequence represented by SEQ ID NO: 75823.

The probes having the nucleotide sequence represented by SEQ ID NOS: 88170, 88171, 88172, 88173, 88174, 88175, 88176, 88177, 88178, 88179, 88180 were selected from the ORFs having the nucleotide sequence represented by SEQ ID NO: 75824.

The probes having the nucleotide sequence represented by SEQ ID NOS: 88181, 88182, 88183, 88184, 88185, 88186, 88187, 88188, 88189, 88190, 88191 were selected from the ORFs having the nucleotide sequence represented by SEQ ID NO: 75825.

The probes having the nucleotide sequence represented by SEQ ID NOS: 88192, 88193, 88194, 88195, 88196, 88197, 88198, 88199, 88200, 88201, 88202 were selected from the ORFs having the nucleotide sequence represented by SEQ ID NO: 75826.

The probes having the nucleotide sequence represented by SEQ ID NOS: 88203, 88204, 88205, 88206, 88207, 88208, 88209, 88210, 88211, 88212, 88213 were selected from the ORFs having the nucleotide sequence represented by SEQ ID NO: 75827.

The probes having the nucleotide sequence represented by SEQ ID NOS: 88214, 88215, 88216, 88217, 88218, 88219, 88220, 88221, 88222, 88223, 88224 were selected from the ORFs having the nucleotide sequence represented by SEQ ID NO: 75828.

The probes having the nucleotide sequence represented by SEQ ID NOS: 88225, 88226, 88227, 88228, 88229, 88230, 88231, 88232, 88233, 88234, 88235 were selected from the ORFs having the nucleotide sequence represented by SEQ ID NO: 75829.

The probes having the nucleotide sequence represented by SEQ ID NOS: 88236, 88237, 88238, 88239, 88240, 88241, 88242, 88243, 88244, 88245, 88246 were selected from the ORFs having the nucleotide sequence represented by SEQ ID NO: 75830.

The probes having the nucleotide sequence represented by SEQ ID NOS: 88247, 88248, 88249, 88250, 88251, 88252, 88253, 88254, 88255, 88256, 88257 were selected from the ORFs having the nucleotide sequence represented by SEQ ID NO: 75831.

The probes having the nucleotide sequence represented by SEQ ID NOS: 88258, 88259, 88260, 88261, 88262, 88263, 88264, 88265, 88266, 88267, 88268 were selected from the ORFs having the nucleotide sequence represented by SEQ ID NO: 75832.

The probes having the nucleotide sequence represented by SEQ ID NOS: 88269, 88270, 88271, 88272, 88273, 88274, 88275, 88276, 88277, 88278, 88279 were selected from the ORFs having the nucleotide sequence represented by SEQ ID NO: 75833.

The probes having the nucleotide sequence represented by SEQ ID NOS: 88280, 88281, 88282, 88283, 88284, 88285, 88286, 88287, 88288, 88289, 88290 were selected from the ORFs having the nucleotide sequence represented by SEQ ID NO: 75834.

The probes having the nucleotide sequence represented by SEQ ID NOS: 88291, 88292, 88293, 88294, 88295, 88296, 88297, 88298, 88299, 88300, 88301 were selected from the ORFs having the nucleotide sequence represented by SEQ ID NO: 75835.

The probes having the nucleotide sequence represented by SEQ ID NOS: 88302, 88303, 88304, 88305, 88306, 88307, 88308, 88309, 88310, 88311, 88312 were selected from the ORFs having the nucleotide sequence represented by SEQ ID NO: 75836.

The probes having the nucleotide sequence represented by SEQ ID NOS: 88313, 88314, 88315, 88316, 88317, 88318, 88319, 88320, 88321, 88322, 88323 were selected from the ORFs having the nucleotide sequence represented by SEQ ID NO: 75837.

The probes having the nucleotide sequence represented by SEQ ID NOS: 88324, 88325, 88326, 88327, 88328, 88329, 88330, 88331, 88332, 88333, 88334 were selected from the ORFs having the nucleotide sequence represented by SEQ ID NO: 75838.

The probes having the nucleotide sequence represented by SEQ ID NOS: 88335, 88336, 88337, 88338, 88339, 88340, 88341, 88342, 88343, 88344, 88345 were selected from the ORFs having the nucleotide sequence represented by SEQ ID NO: 75839.

The probes having the nucleotide sequence represented by SEQ ID NOS: 88346, 88347, 88348, 88349, 88350, 88351, 88352, 88353, 88354, 88355, 88356 were selected from the ORFs having the nucleotide sequence represented by SEQ ID NO: 75840.

The probes having the nucleotide sequence represented by SEQ ID NOS: 88357, 88358, 88359, 88360, 88361, 88362, 88363, 88364, 88365, 88366, 88367 were selected from the ORFs having the nucleotide sequence represented by SEQ ID NO: 75841.

The probes having the nucleotide sequence represented by SEQ ID NOS: 88368, 88369, 88370, 88371, 88372, 88373, 88374, 88375, 88376, 88377, 88378 were selected from the ORFs having the nucleotide sequence represented by SEQ ID NO: 75842.

The probes having the nucleotide sequence represented by SEQ ID NOS: 88379, 88380, 88381, 88382, 88383, 88384, 88385, 88386, 88387, 88388, 88389 were selected from the ORFs having the nucleotide sequence represented by SEQ ID NO: 75843.

The probes having the nucleotide sequence represented by SEQ ID NOS: 88390, 88391, 88392, 88393, 88394, 88395, 88396, 88397, 88398, 88399, 88400 were selected from the ORFs having the nucleotide sequence represented by SEQ ID NO: 75844.

The probes having the nucleotide sequence represented by SEQ ID NOS: 88401, 88402, 88403, 88404, 88405, 88406, 88407, 88408, 88409, 88410, 88411 were selected from the ORFs having the nucleotide sequence represented by SEQ ID NO: 75845.

The probes having the nucleotide sequence represented by SEQ ID NOS: 88412, 88413, 88414, 88415, 88416, 88417, 88418, 88419, 88420, 88421 were selected from the ORFs having the nucleotide sequence represented by SEQ ID NO: 75846.

The probes having the nucleotide sequence represented by SEQ ID NOS: 88422, 88423, 88424, 88425, 88426, 88427, 88428, 88429, 88430, 88431, 88432 were selected from the ORFs having the nucleotide sequence represented by SEQ ID NO: 75847.

The probes having the nucleotide sequence represented by SEQ ID NOS: 88433, 88434, 88435, 88436, 88437, 88438, 88439, 88440, 88441, 88442, 88443 were selected from the ORFs having the nucleotide sequence represented by SEQ ID NO: 75848.

The probes having the nucleotide sequence represented by SEQ ID NOS: 88444, 88445, 88446, 88447, 88448, 88449, 88450, 88451, 88452, 88453, 88454 were selected from the ORFs having the nucleotide sequence-represented by SEQ ID NO: 75849.

The probes having the nucleotide sequence represented by SEQ ID NOS: 88455, 88456, 88457, 88458, 88459, 88460, 88461, 88462, 88463, 88464, 88465 were selected from the ORFs having the nucleotide sequence represented by SEQ ID NO: 75850.

The probes having the nucleotide sequence represented by SEQ ID NOS: 88466, 88467, 88468, 88469, 88470, 88471, 88472, 88473, 88474, 88475, 88476 were selected from the ORFs having the nucleotide sequence represented by SEQ ID NO: 75851.

The probes having the nucleotide sequence represented by SEQ ID NOS: 88477, 88478, 88479, 88480, 88481, 88482, 88483, 88484, 88485, 88486, 88487 were selected from the ORFs having the nucleotide sequence represented by SEQ ID NO: 75852.

The probes having the nucleotide sequence represented by SEQ ID NOS: 88488, 88489, 88490, 88491, 88492, 88493, 88494, 88495, 88496, 88497, 88498 were selected from the ORFs having the nucleotide sequence represented by SEQ ID NO: 75853.

The probes having the nucleotide sequence represented by SEQ ID NOS: 88499, 88500, 88501, 88502, 88503, 88504, 88505, 88506, 88507, 88508, 88509 were selected from the ORFs having the nucleotide sequence represented by SEQ ID NO: 75854.

The probes having the nucleotide sequence represented by SEQ ID NOS: 88510, 88511, 88512, 88513, 88514, 88515, 88516, 88517, 88518, 88519, 88520 were selected from the ORFs having the nucleotide sequence represented by SEQ ID NO: 75855.

The probes-having the nucleotide sequence represented by SEQ ID NOS: 88521, 88522, 88523, 88524, 88525, 88526, 88527, 88528, 88529, 88530, 88531 were selected from the ORFs having the nucleotide sequence represented by SEQ ID NO: 75856.

The probes having the nucleotide sequence represented by SEQ ID NOS: 88532, 88533, 88534, 88535, 88536, 88537, 88538, 88539, 88540, 88541, 88542 were selected from the ORFs having the nucleotide sequence represented by SEQ ID NO: 75857.

The probes having the nucleotide sequence represented by SEQ ID NOS: 88543, 88544, 88545, 88546, 88547, 88548, 88549, 88550, 88551, 88552, 88553 were selected from the ORFs having the nucleotide sequence represented by SEQ ID NO: 75858.

The probes having the nucleotide sequence represented by SEQ ID NOS: 88554, 88555, 88556, 88557, 88558, 88559, 88560, 88561, 88562, 88563, 88564 were selected from the ORFs having the nucleotide sequence represented by SEQ ID NO: 75859.

The probes having the nucleotide sequence represented by SEQ ID NOS: 88565, 88566, 88567, 88568, 88569, 88570, 88571, 88572, 88573, 88574, 88575 were selected from the ORFs having the nucleotide sequence represented by SEQ ID NO: 75860.

The probes having the nucleotide sequence represented by SEQ ID NOS: 88576, 88577, 88578, 88579, 88580, 88581, 88582, 88583, 88584, 88585, 88586 were selected from the ORFs having the nucleotide sequence represented by SEQ ID NO: 75861.

The probes having the nucleotide sequence represented by SEQ ID NOS: 88587, 88588, 88589, 88590, 88591, 88592, 88593, 88594, 88595, 88596, 88597 were selected from the ORFs having the nucleotide sequence represented by SEQ ID NO: 75862.

The probes having the nucleotide sequence represented by SEQ ID NOS: 88598, 88599, 88600, 88601, 88602, 88603, 88604, 88605, 88606, 88607, 88608 were selected from the ORFs having the nucleotide sequence represented by SEQ ID NO: 75863.

The probes having the nucleotide sequence represented by SEQ ID NOS: 88609, 88610, 88611, 88612, 88613, 88614, 88615, 88616, 88617, 88618, 88619 were selected from the ORFs having the nucleotide sequence represented by SEQ ID NO: 75864.

The probes having the nucleotide sequence represented by SEQ ID NOS: 88620, 88621, 88622, 88623, 88624, 88625, 88626, 88627, 88628, 88629, 88630 were selected from the ORFs having the nucleotide sequence represented by SEQ ID NO: 75865.

The probes having the nucleotide sequence represented by SEQ ID NOS: 88631, 88632, 88633, 88634, 88635, 88636, 88637, 88638, 88639, 88640, 88641 were selected from the ORFs having the nucleotide sequence represented by SEQ ID NO: 75866.

The probes having the nucleotide sequence represented by SEQ ID NOS: 88642, 88643, 88644, 88645, 88646, 88647, 88648, 88649, 88650, 88651, 88652 were selected from the ORFs having the nucleotide sequence represented by SEQ ID NO: 75867.

The probes having the nucleotide sequence represented by SEQ ID NOS: 88653, 88654, 88655, 88656, 88657, 88658, 88659, 88660, 88661, 88662, 88663 were selected from the ORFs having the nucleotide sequence represented by SEQ ID NO: 75868.

The probes having the nucleotide sequence represented by SEQ ID NOS: 88664, 88665, 88666, 88667, 88668, 88669, 88670, 88671, 88672, 88673, 88674 were selected from the ORFs having the nucleotide sequence represented by SEQ ID NO: 75869.

The probes having the nucleotide sequence represented by SEQ ID NOS: 88675, 88676, 88677, 88678, 88679, 88680, 88681, 88682, 88683, 88684, 88685 were selected from the ORFs having the nucleotide sequence represented by SEQ ID NO: 75870.

The probes having the nucleotide sequence represented by SEQ ID NOS: 88686, 88687, 88688, 88689, 88690, 88691, 88692, 88693, 88694, 88695, 88696 were selected from the ORFs having the nucleotide sequence represented by SEQ ID NO: 75871.

The probes having the nucleotide sequence represented by SEQ ID NOS: 88697, 88698, 88699, 88700, 88701, 88702, 88703, 88704, 88705, 88706, 88707 were selected from the ORFs having the nucleotide sequence represented by SEQ ID NO: 75872.

The probes having the nucleotide sequence represented by SEQ ID NOS: 88708, 88709, 88710, 88711, 88712, 88713, 88714, 88715, 88716, 88717, 88718 were selected from the ORFs having the nucleotide sequence represented by SEQ ID NO: 75873.

The probes having the nucleotide sequence represented by SEQ ID NOS: 88719, 88720, 88721, 88722, 88723, 88724, 88725, 88726, 88727, 88728, 88729 were selected from the ORFs having the nucleotide sequence represented by SEQ ID NO: 75874.

The probes having the nucleotide sequence represented by SEQ ID NOS: 88730, 88731, 88732, 88733, 88734, 88735, 88736, 88737, 88738, 88739, 88740 were selected from the ORFs having the nucleotide sequence represented by SEQ ID NO: 75875.

The probes having the nucleotide sequence represented by SEQ ID NOS: 88741, 88742, 88743, 88744, 88745, 88746, 88747, 88748, 88749, 88750, 88751 were selected from the ORFs having the nucleotide sequence represented by SEQ ID NO: 75876.

The probes having the nucleotide sequence represented by SEQ ID NOS: 88752, 88753, 88754, 88755, 88756, 88757, 88758, 88759, 88760, 88761, 88762 were selected from the ORFs having the nucleotide sequence represented by SEQ ID NO: 75877.

The probes having the nucleotide sequence represented by SEQ ID NOS: 88763, 88764, 88765, 88766, 88767, 88768, 88769, 88770, 88771, 88772, 88773 were selected from the ORFs having the nucleotide sequence represented by SEQ ID NO: 75878.

The probes having the nucleotide sequence represented by SEQ ID NOS: 88774, 88775, 88776, 88777, 88778, 88779, 88780, 88781, 88782, 88783, 88784 were selected from the ORFs having the nucleotide sequence represented by SEQ ID NO: 75879.

The probes having the nucleotide sequence represented by SEQ ID NOS: 88785, 88786, 88787, 88788, 88789, 88790, 88791, 88792, 88793, 88794, 88795 were selected from the ORFs having the nucleotide sequence represented by SEQ ID NO: 75880.

The probes having the nucleotide sequence represented by SEQ ID NOS: 88796, 88797, 88798, 88799, 88800, 88801, 88802, 88803, 88804, 88805, 88806 were selected from the ORFs having the nucleotide sequence represented by SEQ ID NO: 75881.

The probes having the nucleotide sequence represented by SEQ ID NOS: 88807, 88808, 88809, 88810, 88811, 88812, 88813, 88814, 88815, 88816, 88817 were selected from the ORFs having the nucleotide sequence represented by SEQ ID NO: 75882.

The probes having the nucleotide sequence represented by SEQ ID NOS: 88818, 88819, 88820, 88821, 88822, 88823, 88824, 88825, 88826, 88827, 88828 were selected from the ORFs having the nucleotide sequence represented by SEQ ID NO: 75883.

The probes having the nucleotide sequence represented by SEQ ID NOS: 88829, 88830, 88831, 88832, 88833, 88834, 88835, 88836, 88837, 88838, 88839 were selected from the ORFs having the nucleotide sequence represented by SEQ ID NO: 75884.

The probes having the nucleotide sequence represented by SEQ ID NOS: 88840, 88841, 88842, 88843, 88844, 88845, 88846, 88847, 88848, 88849, 88850 were selected from the ORFs having the nucleotide sequence represented by SEQ ID NO: 75885.

The probes having the nucleotide sequence represented by SEQ ID NOS: 88851, 88852, 88853, 88854, 88855, 88856, 88857, 88858, 88859, 88860, 88861 were selected from the ORFs having the nucleotide sequence represented by SEQ ID NO: 75886.

The probes having the nucleotide sequence represented by SEQ ID NOS: 88862, 88863, 88864, 88865, 88866, 88867, 88868, 88869, 88870, 88871, 88872 were selected from the ORFs having the nucleotide sequence represented by SEQ ID NO: 75887.

The probes having the nucleotide sequence represented by SEQ ID NOS: 88873, 88874, 88875, 88876, 88877, 88878, 88879, 88880, 88881, 88882, 88883 were selected from the ORFs having the nucleotide sequence represented by SEQ ID NO: 75888.

The probes having the nucleotide sequence represented by SEQ ID NOS: 88884, 88885, 88886, 88887, 88888, 88889, 88890, 88891, 88892, 88893, 88894 were selected from the ORFs having the nucleotide sequence represented by SEQ ID NO: 75889.

The probes having the nucleotide sequence represented by SEQ ID NOS: 88895, 88896, 88897, 88898, 88899, 88900, 88901, 88902, 88903, 88904, 88905 were selected from the ORFs having the nucleotide sequence represented by SEQ ID NO: 75890.

The probes having the nucleotide sequence represented by SEQ ID NOS: 88906, 88907, 88908, 88909, 88910, 88911, 88912, 88913, 88914, 88915, 88916 were selected from the ORFs having the nucleotide sequence represented by SEQ ID NO: 75891.

The probes having the nucleotide sequence represented by SEQ ID NOS: 88917, 88918, 88919, 88920, 88921, 88922, 88923,. 88924, 88925, 88926, 88927 were selected from the ORFs having the nucleotide sequence represented by SEQ ID NO: 75892.

The probes having the nucleotide sequence represented by SEQ ID NOS: 88928, 88929, 88930, 88931, 88932, 88933, 88934, 88935, 88936, 88937, 88938 were selected from the ORFs having the nucleotide sequence represented by SEQ ID NO: 75893.

The probes having the nucleotide sequence represented by SEQ ID NOS: 88939, 88940, 88941, 88942, 88943, 88944, 88945, 88946, 88947, 88948, 88949 were selected from the ORFs having the nucleotide sequence represented by SEQ ID NO: 75894.

The probes having the nucleotide sequence represented by SEQ ID NOS: 88950, 88951, 88952, 88953, 88954, 88955, 88956, 88957, 88958, 88959, 88960 were selected from the ORFs having the nucleotide sequence represented by SEQ ID NO: 75895.

The probes having the nucleotide sequence represented by SEQ ID NOS: 88961, 88962, 88963, 88964, 88965, 88966, 88967, 88968, 88969, 88970, 88971 were selected from the ORFs having the nucleotide sequence represented by SEQ ID NO: 75896.

The probes having the nucleotide sequence represented by SEQ ID NOS: 88972, 88973, 88974, 88975, 88976, 88977, 88978, 88979, 88980, 88981, 88982 were selected from the ORFs having the nucleotide sequence represented by SEQ ID NO: 75897.

The probes having the nucleotide sequence represented by SEQ ID NOS: 88983, 88984, 88985, 88986, 88987, 88988, 88989, 88990, 88991, 88992, 88993 were selected from the ORFs having the nucleotide sequence represented by SEQ ID NO: 75898.

The probes having the nucleotide sequence represented by SEQ ID NOS: 88994, 88995, 88996, 88997, 88998, 88999, 89000, 89001, 89002, 89003, 89004 were selected from the ORFs having the nucleotide sequence represented by SEQ ID NO: 75899.

The probes having the nucleotide sequence represented by SEQ ID NOS: 89005, 89006, 89007, 89008, 89009, 89010, 89011, 89012, 89013, 89014, 89015 were selected from the ORFs having the nucleotide sequence represented by SEQ ID NO: 75900.

The probes having the nucleotide sequence represented by SEQ ID NOS: 89016, 89017, 89018, 89019, 89020, 89021, 89022, 89023, 89024, 89025, 89026 were selected from the ORFs having the nucleotide sequence represented by SEQ ID NO: 75901.

The probes having the nucleotide sequence represented by SEQ ID NOS: 89027, 89028, 89029, 89030, 89031, 89032, 89033, 89034, 89035, 89036, 89037 were selected from the ORFs having the nucleotide sequence represented by SEQ ID NO: 75902.

The probes having the nucleotide sequence represented by SEQ ID NOS: 89038, 89039, 89040, 89041, 89042, 89043, 89044, 89045, 89046, 89047, 89048 were selected from the ORFs having the nucleotide sequence represented by SEQ ID NO: 75903.

The probes having the nucleotide sequence represented by SEQ ID NOS: 89049, 89050, 89051, 89052, 89053, 89054, 89055, 89056, 89057, 89058, 89059 were selected from the ORFs having the nucleotide sequence represented by SEQ ID NO: 75904.

The probes having the nucleotide sequence represented by SEQ ID NOS: 89060, 89061, 89062, 89063, 89064, 89065, 89066, 89067, 89068, 89069, 89070 were selected from the ORFs having the nucleotide sequence represented by SEQ ID NO: 75905.

The probes having the nucleotide sequence represented by SEQ ID NOS: 89071, 89072, 89073, 89074, 89075, 89076, 89077, 89078, 89079, 89080, 89081 were selected from the ORFs having the nucleotide sequence represented by SEQ ID NO: 75906.

The probes having the nucleotide sequence represented by SEQ ID NOS: 89082, 89083, 89084, 89085, 89086, 89087, 89088, 89089, 89090, 89091, 89092 were selected from the ORFs having the nucleotide sequence represented by SEQ ID NO: 75907.

The probes having the nucleotide sequence represented by SEQ ID NOS: 89093, 89094, 89095, 89096, 89097, 89098, 89099, 89100, 89101, 89102, 89103 were selected from the ORFs having the nucleotide sequence represented by SEQ ID NO: 75908.

The probes having the nucleotide sequence represented by SEQ ID NOS: 89104, 89105, 89106, 89107, 89108, 89109, 89110, 89111, 89112, 89113, 89114 were selected from the ORFs having the nucleotide sequence represented by SEQ ID NO: 75909.

The probes having the nucleotide sequence represented by SEQ ID NOS: 89115, 89116, 89117, 89118, 89119, 89120, 89121, 89122, 89123, 89124, 89125 were selected from the ORFs having the nucleotide sequence represented by SEQ ID NO: 75910.

The probes having the nucleotide sequence represented by SEQ ID NOS: 89126, 89127, 89128, 89129, 89130, 89131, 89132, 89133, 89134, 89135, 89136 were selected from the ORFs having the nucleotide sequence represented by SEQ ID NO: 75911.

The probes having the nucleotide sequence represented by SEQ ID NOS: 89137, 89138, 89139, 89140, 89141, 89142, 89143, 89144, 89145, 89146, 89147 were selected from the ORFs having the nucleotide sequence represented by SEQ ID NO: 75912.

The probes having the nucleotide sequence represented by SEQ ID NOS: 89148, 89149, 89150, 89151, 89152, 89153, 89154, 89155, 89156, 89157, 89158 were selected from the ORFs having the nucleotide sequence represented by SEQ ID NO: 75913.

The probes having the nucleotide sequence represented by SEQ ID NOS: 89159, 89160, 89161, 89162, 89163, 89164, 89165, 89166, 89167, 89168, 89169 were selected from the ORFs having the nucleotide sequence represented by SEQ ID NO: 75914.

The probes having the nucleotide sequence represented by SEQ ID NOS: 89170, 89171, 89172, 89173, 89174, 89175, 89176, 89177, 89178, 89179, 89180 were selected from the ORFs having the nucleotide sequence represented by SEQ ID NO: 75915.

The probes having the nucleotide sequence represented by SEQ ID NOS: 89181, 89182, 89183, 89184, 89185, 89186, 89187, 89188, 89189, 89190, 89191 were selected from the ORFs having the nucleotide sequence represented by SEQ ID. NO: 75916.

The probes having the nucleotide sequence represented by SEQ ID NOS: 89192, 89193, 89194, 89195, 89196, 89197, 89198, 89199, 89200, 89201, 89202 were selected from the ORFs having the nucleotide sequence represented by SEQ ID NO: 75917.

The probes having the nucleotide sequence represented by SEQ ID NOS: 89203, 89204, 89205, 89206, 89207, 89208, 89209, 89210, 89211, 89212, 89213 were selected from the ORFs having the nucleotide sequence represented by SEQ ID NO: 75918.

The probes having the nucleotide sequence represented by SEQ ID NOS: 89214, 89215, 89216, 89217, 89218, 89219, 89220, 89221, 89222, 89223, 89224 were selected from the ORFs having the nucleotide sequence represented by SEQ ID. NO: 75919.

The probes having the nucleotide sequence represented by SEQ ID NOS: 89225, 89226, 89227, 89228, 89229, 89230, 89231, 89232, 89233, 89234, 89235 were selected from the ORFs having the nucleotide sequence represented by SEQ ID NO: 75920.

The probes having the nucleotide sequence represented by SEQ ID NOS: 89236, 89237, 89238, 89239, 89240, 89241, 89242, 89243, 89244, 89245, 89246 were selected from the ORFs having the nucleotide sequence represented by SEQ ID NO: 75921.

The probes having the nucleotide sequence represented by SEQ ID NOS: 89247, 89248, 89249, 89250, 89251, 89252, 89253, 89254, 89255, 89256, 89257 were selected from the ORFs having the nucleotide sequence represented by SEQ ID NO: 75922.

The probes having the nucleotide sequence represented by SEQ ID NOS: 89258, 89259, 89260, 89261, 89262, 89263, 89264, 89265, 89266, 89267, 89268 were selected from the ORFs having the nucleotide sequence represented by SEQ ID NO: 75923.

The probes having the nucleotide sequence represented by SEQ ID NOS: 89269, 89270, 89271, 89272, 89273, 89274, 89275, 89276, 89277, 89278, 89279 were selected from the ORFs having the nucleotide sequence represented by SEQ ID NO: 75924.

The probes having the nucleotide sequence represented by SEQ ID NOS: 89280, 89281, 89282, 89283, 89284, 89285, 89286, 89287, 89288, 89289, 89290 were selected from the ORFs having the nucleotide sequence represented by SEQ ID NO: 75925.

The probes having the nucleotide sequence represented by SEQ ID NOS: 89291, 89292, 89293, 89294, 89295, 89296, 89297, 89298, 89299, 89300, 89301 were selected from the ORFs having the nucleotide sequence represented by SEQ ID NO: 75926.

The probes having the nucleotide sequence represented by SEQ ID NOS: 89302, 89303, 89304, 89305, 89306, 89307, 89308, 89309, 89310, 89311, 89312 were selected from the ORFs having the nucleotide sequence represented by SEQ ID NO: 75927.

The probes having the nucleotide sequence represented by SEQ ID NOS: 89313, 89314, 89315, 89316, 89317, 89318, 89319, 89320, 89321, 89322, 89323 were selected from the ORFs having the nucleotide sequence represented by SEQ ID NO: 75928.

The probes having the nucleotide sequence represented by SEQ ID NOS: 89324, 89325, 89326, 89327, 89328, 89329, 89330, 89331, 89332, 89333, 89334 were selected from the ORFs having the nucleotide sequence represented by SEQ ID NO: 75929.

The probes having the nucleotide sequence represented by SEQ ID NOS: 89335, 89336, 89337, 89338, 89339, 89340, 89341, 89342, 89343, 89344, 89345 were selected from the ORFs having the nucleotide sequence represented by SEQ ID NO: 75930.

The probes having the nucleotide sequence represented by SEQ ID NOS: 89346, 89347, 89348, 89349, 89350, 89351, 89352, 89353, 89354, 89355, 89356 were selected from the ORFs having the nucleotide sequence represented by SEQ ID NO: 75931.

The probes having the nucleotide sequence represented by SEQ ID NOS: 89357, 89358, 89359, 89360, 89361, 89362, 89363, 89364, 89365, 89366, 89367 were selected from the ORFs having the nucleotide sequence represented by SEQ ID NO: 75932.

The probes having the nucleotide sequence represented by SEQ ID NOS: 89368, 89369, 89370, 89371, 89372, 89373, 89374, 89375, 89376, 89377, 89378 were selected from the ORFs having the nucleotide sequence represented by SEQ ID NO: 75933.

The probes having the nucleotide sequence represented by SEQ ID NOS: 89379, 89380, 89381, 89382, 89383, 89384, 89385, 89386, 89387, 89388, 89389 were selected from the ORFs having the nucleotide sequence represented by SEQ ID NO: 75934.

The probes having the nucleotide sequence represented by SEQ ID NOS: 89390, 89391, 89392, 89393, 89394, 89395, 89396, 89397, 89398, 89399, 89400 were selected from the ORFs having the nucleotide sequence represented by SEQ ID NO: 75935.

The probes having the nucleotide sequence represented by SEQ ID NOS: 89401, 89402, 89403, 89404, 89405, 89406, 89407, 89408, 89409, 89410, 89411 were selected from the ORFs having the nucleotide sequence represented by SEQ ID NO: 75936.

The probes having the nucleotide sequence represented by SEQ ID NOS: 89412, 89413, 89414, 89415, 89416, 89417, 89418, 89419, 89420, 89421, 89422 were selected from the ORFs having the nucleotide sequence represented by SEQ ID NO: 75937.

The probes having the nucleotide sequence represented by SEQ ID NOS: 89423, 89424, 89425, 89426, 89427, 89428, 89429, 89430, 89431, 89432, 89433 were selected from the ORFs having the nucleotide sequence represented by SEQ ID NO: 75938.

The probes having the nucleotide sequence represented by SEQ ID NOS: 89434, 89435, 89436, 89437, 89438, 89439, 89440, 89441, 89442, 89443, 8944.4 were selected from the ORFs having the nucleotide sequence represented by SEQ ID NO: 75939.

The probes having the nucleotide sequence represented by SEQ ID NOS: 89445, 89446, 89447, 89448, 89449, 89450, 89451, 89452, 89453, 89454, 89455 were selected from the ORFs having the nucleotide sequence represented by SEQ ID NO: 75940.

The probes having the nucleotide sequence represented by SEQ ID NOS: 89456, 89457, 89458, 89459, 89460, 89461, 89462, 89463, 89464, 89465, 89466 were selected from the ORFs having the nucleotide sequence represented by SEQ ID NO: 75941.

The probes having the nucleotide sequence represented by SEQ ID NOS: 89467, 89468, 89469, 89470, 89471, 89472, 89473, 89474, 89475, 89476, 89477 were selected from the ORFs having the nucleotide sequence represented by SEQ ID NO: 75942.

The probes having the nucleotide sequence represented by SEQ ID NOS: 89478, 89479, 89480, 89481, 89482, 89483, 89484, 89485, 89486, 89487, 89488 were selected from the ORFs having the nucleotide sequence represented by SEQ ID NO: 75943.

The probes having the nucleotide sequence represented by SEQ ID NOS: 89489, 89490, 89491, 89492, 89493, 89494, 89495, 89496, 89497, 89498, 89499 were selected from the ORFs having the nucleotide sequence represented by SEQ ID NO: 75944.

The probes having the nucleotide sequence represented by. SEQ ID NOS: 89500, 89501, 89502, 89503, 89504, 89505, 89506, 89507, 89508, 89509, 89510 were selected from the ORFs having the nucleotide sequence represented by SEQ ID NO: 75945.

The probes having the nucleotide sequence represented by SEQ ID NOS: 89511, 89512, 89513, 89514, 89515, 89516, 89517, 89518, 89519, 89520, 89521 were selected from the ORFs having the nucleotide sequence represented by SEQ ID NO: 75946.

The probes having the nucleotide sequence represented by SEQ ID NOS: 89522, 89523, 89524, 89525, 89526, 89527, 89528, 89529, 89530, 89531, 89532 were selected from the ORFs having the nucleotide sequence represented by SEQ ID NO: 75947.

The probes having the nucleotide sequence represented by SEQ ID NOS: 89533, 89534, 89535, 89536, 89537, 89538, 89539, 89540, 89541, 89542, 89543 were selected from the ORFs having the nucleotide sequence represented by SEQ ID NO: 75948.

The probes having the nucleotide sequence represented by SEQ ID NOS: 89544, 89545, 89546, 89547, 89548, 89549, 89550, 89551, 89552, 89553, 89554 were selected from the ORFs having the nucleotide sequence represented by SEQ ID. NO: 75949.

The probes having the nucleotide sequence represented by SEQ ID NOS: 89555, 89556, 89557, 89558, 89559, 89560, 89561, 89562, 89563, 89564, 89565 were selected from the ORFs having the nucleotide sequence represented by SEQ ID NO: 75950.

The probes having the nucleotide sequence represented by SEQ ID NOS: 89566, 89567, 89568, 89569, 89570, 89571, 89572, 89573, 89574, 89575, 89576 were selected from the ORFs having the nucleotide sequence represented by SEQ ID NO: 75951.

The probes having the nucleotide sequence represented by SEQ ID NOS: 89577, 89578, 89579, 89580, 89581, 89582, 89583, 89584, 89585, 89586, 89587 were selected from the ORFs having the nucleotide sequence represented by SEQ ID NO: 75952.

The probes having the nucleotide sequence represented by SEQ ID NOS: 89588, 89589, 89590, 89591, 89592, 89593, 89594, 89595, 89596, 89597, 89598 were selected from the ORFs having the nucleotide sequence represented by SEQ ID NO: 75953.

The probes having the nucleotide sequence represented by SEQ ID NOS: 89599, 89600, 89601, 89602, 89603, 89604, 89605, 89606, 89607, 89608, 89609 were selected from the ORFs having the nucleotide sequence represented by SEQ ID NO: 75954.

The probes having the nucleotide sequence represented by SEQ ID NOS: 89610, 89611, 89612, 89613, 89614, 89615, 89616, 89617, 89618, 89619, 89620 were selected from the ORFs having the nucleotide sequence represented by SEQ ID NO: 75955.

The probes having the nucleotide sequence represented by SEQ ID NOS: 89621, 89622, 89623, 89624, 89625, 89626, 89627, 89628, 89629, 89630, 89631 were selected from the ORFs having the nucleotide sequence represented by SEQ ID NO: 75956.

The probes having the nucleotide sequence represented by SEQ ID NOS: 89632, 89633, 89634, 89635, 89636, 89637, 89638, 89639, 89640, 89641, 89642 were selected from the ORFs having the nucleotide sequence represented by SEQ ID NO: 75957.

The probes having the nucleotide sequence represented by SEQ ID NOS: 89643, 89644, 89645, 89646, 89647, 89648, 89649, 89650, 89651, 89652, 89653 were selected from the ORFs having the nucleotide sequence represented by SEQ ID NO: 75958.

The probes having the nucleotide sequence represented by SEQ ID NOS: 89654, 89655, 89656, 89657, 89658, 89659, 89660, 89661, 89662, 89663, 89664 were selected from the ORFs having the nucleotide sequence represented by SEQ ID NO: 75959.

The probes having the nucleotide sequence represented by SEQ ID NOS: 89665, 89666, 89667, 89668, 89669, 89670, 89671, 89672, 89673, 89674, 89675 were selected from the ORFs having the nucleotide sequence represented by SEQ ID NO: 75960.

The probes having the nucleotide sequence represented by SEQ ID NOS: 89676, 89677, 89678, 89679, 89680, 89681, 89682, 89683, 89684, 89685, 89686 were selected from the ORFs having the nucleotide sequence represented by SEQ ID NO: 75961.

The probes having the nucleotide sequence represented by SEQ ID NOS: 89687, 89688, 89689, 89690, 89691, 89692, 89693, 89694, 89695, 89696, 89697 were selected from the ORFs having the nucleotide sequence represented by SEQ ID NO: 75962.

The probes having the nucleotide sequence represented by SEQ ID NOS: 89698, 89699, 89700, 89701, 89702, 89703, 89704, 89705, 89706, 89707, 89708 were selected from the ORFs having the nucleotide sequence represented by SEQ ID NO: 75963.

The probes having the nucleotide sequence represented by SEQ ID NOS: 89709, 89710, 89711, 89712, 89713, 89714, 89715, 89716, 89717, 89718, 89719 were selected from the ORFs having the nucleotide sequence represented by SEQ ID NO: 75964.

The probes having the nucleotide sequence represented by SEQ ID NOS: 89720, 89721, 89722, 89723, 89724, 89725, 89726, 89727, 89728, 89729, 89730 were selected from the ORFs having the nucleotide sequence represented by SEQ ID NO: 75965.

The probes having the nucleotide sequence represented by SEQ ID NOS: 89731, 89732, 89733, 89734, 89735, 89736, 89737, 89738, 89739, 89740, 89741 were selected from the ORFs having the nucleotide sequence represented by SEQ ID NO: 75966.

The probes having the nucleotide sequence represented by SEQ ID NOS: 89742, 89743, 89744, 89745, 89746, 89747, 89748, 89749, 89750, 89751, 89752 were selected from the ORFs having the nucleotide sequence represented by SEQ ID NO: 75967.

The probes having the nucleotide sequence represented by SEQ ID NOS: 89753, 89754, 89755, 89756, 89757, 89758, 89759, 89760, 89761, 89762, 89763 were selected from the ORFs having the nucleotide sequence represented by SEQ ID NO: 75968.

The probes having the nucleotide sequence represented by SEQ ID NOS: 89764, 89765, 89766, 89767, 89768, 89769, 89770, 89771, 89772, 89773, 89774 were selected from the ORFs having the nucleotide sequence represented by SEQ ID NO: 75969.

The probes having the nucleotide sequence represented by SEQ ID NOS: 89775, 89776, 89777, 89778, 89779, 89780, 89781, 89782, 89783, 89784, 89785 were selected from the ORFs having the nucleotide sequence represented by SEQ ID NO: 75970.

The probes having the nucleotide sequence represented by SEQ ID NOS: 89786, 89787, 89788, 89789, 89790, 89791, 89792, 89793, 89794, 89795, 89796 were selected from the ORFs having the nucleotide sequence represented by SEQ ID NO: 75971.

The probes having the nucleotide sequence represented by SEQ ID NOS: 89797, 89798, 89799, 89800, 89801, 89802, 89803, 89804, 89805, 89806, 89807 were selected from the ORFs having the nucleotide sequence represented by SEQ ID NO: 75972.

The probes having the nucleotide sequence represented by SEQ ID NOS: 89808, 89809, 89810, 89811, 89812, 89813, 89814, 89815, 89816, 89817, 89818 were selected from the ORFs having the nucleotide sequence represented by SEQ ID NO: 75973.

The probes having the nucleotide sequence represented by SEQ ID NOS: 89819, 89820, 89821, 89822, 89823, 89824, 89825, 89826, 89827, 89828, 89829 were selected from the ORFs having the nucleotide sequence represented by SEQ ID NO: 75974.

The probes having the nucleotide sequence represented by SEQ ID NOS: 89830, 89831, 89832, 89833, 89834, 89835, 89836, 89837, 89838, 89839, 89840 were selected from the ORFs having the nucleotide sequence represented by SEQ ID NO: 75975.

The probes having the nucleotide sequence represented by SEQ ID NOS: 89841, 89842, 89843, 89844, 89845, 89846, 89847, 89848, 89849, 89850, 89851 were selected from the ORFs having the nucleotide sequence represented by SEQ ID NO: 75976.

The probes having the nucleotide sequence represented by SEQ ID NOS: 89852, 89853, 89854, 89855, 89856, 89857, 89858, 89859, 89860, 89861, 89862 were selected from the ORFs having the nucleotide sequence represented by SEQ ID NO: 75977.

The probes having the nucleotide sequence represented by SEQ ID NOS: 89863, 89864, 89865, 89866, 89867, 89868, 89869, 89870, 89871, 89872, 89873 were selected from the ORFs having the nucleotide sequence represented by SEQ ID NO: 75978.

The probes having the nucleotide sequence represented by SEQ ID NOS: 89874, 89875, 89876, 89877, 89878, 89879, 89880, 89881, 89882, 89883, 89884 were selected from the ORFs having the nucleotide sequence represented by SEQ ID NO: 75979.

The probes having the nucleotide sequence represented by SEQ ID NOS: 89885, 89886, 89887, 89888, 89889, 89890, 89891, 89892, 89893, 89894, 89895 were selected from the ORFs having the nucleotide sequence represented by SEQ ID NO: 75980.

The probes having the nucleotide sequence represented by SEQ ID NOS: 89896, 89897, 89898, 89899, 89900, 89901, 89902, 89903, 89904, 89905, 89906 were selected from the ORFs having the nucleotide sequence represented by SEQ ID NO: 75981.

The probes having the nucleotide sequence represented by SEQ ID NOS: 89907, 89908, 89909, 89910, 89911, 89912, 89913, 89914, 89915, 89916, 89917 were selected from the ORFs having the nucleotide sequence represented by SEQ ID NO: 75982.

The probes having the nucleotide sequence represented by SEQ ID NOS: 89918, 89919, 89920, 89921, 89922, 89923, 89924, 89925, 89926, 89927, 89928 were selected from the ORFs having the nucleotide sequence represented by SEQ ID NO: 75983.

The probes having the nucleotide sequence represented by SEQ ID NOS: 89929, 89930, 89931, 89932, 89933, 89934, 89935, 89936, 89937, 89938, 89939 were selected from the ORFs having the nucleotide sequence represented by SEQ ID NO: 75984.

The probes having the nucleotide sequence represented by SEQ ID NOS: 89940, 89941, 89942, 89943, 89944, 89945, 89946, 89947, 89948, 89949, 89950 were selected from the ORFs having the nucleotide sequence represented by SEQ ID NO: 75985.

The probes having the nucleotide sequence represented by SEQ ID NOS: 89951, 89952, 89953, 89954, 89955, 89956, 89957, 89958, 89959, 89960, 89961 were selected from the ORFs having the nucleotide sequence represented by SEQ ID NO: 75986.

The probes having the nucleotide sequence represented by SEQ ID NOS: 89962, 89963, 89964, 89965, 89966, 89967, 89968, 89969, 89970, 89971, 89972 were selected from the ORFs having the nucleotide sequence represented by SEQ ID NO: 75987.

The probes having the nucleotide sequence represented by SEQ ID NOS: 89973, 89974, 89975, 89976, 89977, 89978, 89979, 89980, 89981, 89982, 89983 were selected from the ORFs having the nucleotide sequence represented by SEQ ID NO: 75988.

The probes having the nucleotide sequence represented by SEQ ID NOS: 89984, 89985, 89986, 89987, 89988, 89989, 89990, 89991, 89992, 89993, 89994 were selected from the ORFs having the nucleotide sequence represented by SEQ ID NO: 75989.

The probes having the nucleotide sequence represented by SEQ ID NOS: 89995, 89996, 89997, 89998, 89999, 90000, 90001, 90002, 90003, 90004, 90005 were selected from the ORFs having the nucleotide sequence represented by SEQ ID NO: 75990.

The probes having the nucleotide sequence represented by SEQ ID NOS: 90006, 90007, 90008, 90009, 90010, 90011, 90012, 90013, 90014, 90015, 90016 were selected from the ORFs having the nucleotide sequence represented by SEQ ID NO: 75991.

The probes having the nucleotide sequence represented by SEQ ID NOS: 90017, 90018, 90019, 90020, 90021, 90022, 90023, 90024, 90025, 90026, 90027 were selected from the ORFs having the nucleotide sequence represented by SEQ ID NO: 75992.

The probes having the nucleotide sequence represented by SEQ ID NOS: 90028, 90029, 90030, 90031, 90032, 90033, 90034, 90035, 90036, 90037, 90038, 90039, 90040, 90041, 90042, 90043, 90044, 90045, 90046, 90047, 90048, 90049 were selected from the ORFs having the nucleotide sequence represented by SEQ ID NO: 75993.

The probes having the nucleotide sequence represented by SEQ ID NOS: 90050, 90051, 90052, 90053, 90054, 90055, 90056, 90057, 90058, 90059, 90060 were selected from the ORFs having the nucleotide sequence represented by SEQ ID NO: 75994.

The probes having the nucleotide sequence represented by SEQ ID NOS: 90061, 90062, 90063, 90064, 90065, 90066, 90067, 90068, 90069, 90070, 90071 were selected from the ORFs having the nucleotide sequence represented by SEQ ID NO: 75995.

The probes having the nucleotide sequence represented by SEQ ID NOS: 90072, 90073, 90074, 90075, 90076, 90077, 90078, 90079, 90080, 90081, 90082 were selected from the ORFs having the nucleotide sequence represented by SEQ ID NO: 75996.

The probes having the nucleotide sequence represented by SEQ ID NOS: 90083, 90084, 90085, 90086, 90087, 90088, 90089, 90090, 90091, 90092, 90093 were selected from the ORFs having the nucleotide sequence represented by SEQ ID NO: 75997.

The probes having the nucleotide sequence represented by SEQ ID NOS: 90094, 90095, 90096, 90097, 90098, 90099, 90100, 90101, 90102, 90103, 90104 were selected from the ORFs having the nucleotide sequence represented by SEQ ID NO: 75998.

The probes having the nucleotide sequence represented by SEQ ID NOS: 90105, 90106, 90107, 90108, 90109, 90110, 90111, 90112, 90113, 90114, 90115 were selected from the ORFs having the nucleotide sequence represented by SEQ ID NO: 75999.

The probes having the nucleotide sequence represented by SEQ ID NOS: 90116, 90117, 90118, 90119, 90120, 90121, 90122, 90123, 90124, 90125, 90126 were selected from the ORFs having the nucleotide sequence represented by SEQ ID NO: 76000.

The probes having the nucleotide sequence represented by SEQ ID NOS: 90127, 90128, 90129, 90130, 90131, 90132, 90133, 90134, 90135, 90136, 90137 were selected from the ORFs having the nucleotide sequence represented by SEQ ID NO: 76001.

The probes having the nucleotide sequence represented by SEQ ID NOS: 90138, 90139, 90140, 90141, 90142, 90143, 90144, 90145, 90146, 90147, 90148 were selected from the ORFs having the nucleotide sequence represented by SEQ ID NO: 76002.

The probes having the nucleotide sequence represented by SEQ ID NOS: 90149, 90150, 90151, 90152, 90153, 90154, 90155, 90156, 90157, 90158, 90159 were selected from the ORFs having the nucleotide sequence represented by SEQ ID NO: 76003.

The probes having the nucleotide sequence represented by SEQ ID NOS: 90160, 90161, 90162, 90163, 90164, 90165, 90166, 90167, 90168, 90169, 90170 were selected from the ORFs having the nucleotide sequence represented by SEQ ID NO: 76004.

The probes having the nucleotide sequence represented by SEQ ID NOS: 90171, 90172, 90173, 90174, 90175, 90176, 90177, 90178, 90179, 90180, 90181 were selected from the ORFs having the nucleotide sequence represented by SEQ ID NO: 76005.

The probes having the nucleotide sequence represented by SEQ ID NOS: 90182, 90183, 90184, 90185, 90186, 90187, 90188, 90189, 90190, 90191, 90192 were selected from the ORFs having the nucleotide sequence represented by SEQ ID NO: 76006.

The probes having the nucleotide sequence represented by SEQ ID NOS: 90193, 90194, 90195, 90196, 90197, 90198, 90199, 90200, 90201, 90202, 90203 were selected from the ORFs having the nucleotide sequence represented by SEQ ID NO: 76007.

The probes having the nucleotide sequence represented by SEQ ID NOS: 90204, 90205, 90206, 90207, 90208, 90209, 90210, 90211, 90212, 90213, 90214 were selected from the ORFs having the nucleotide sequence represented by SEQ ID NO: 76008.

The probes having the nucleotide sequence represented by SEQ ID NOS: 90215, 90216, 90217, 90218, 90219, 90220, 90221, 90222, 90223, 90224, 90225 were selected from the ORFs having the nucleotide sequence represented by SEQ ID NO: 76009.

The probes having the nucleotide sequence represented by SEQ ID NOS: 90226, 90227, 90228, 90229, 90230, 90231, 90232, 90233, 90234, 90235, 90236 were selected from the ORFs having the nucleotide sequence represented by SEQ ID NO: 76010.

The probes having the nucleotide sequence represented by SEQ ID NOS: 90237, 90238, 90239, 90240, 90241, 90242, 90243, 90244, 90245, 90246, 90247 were selected from the ORFs having the nucleotide sequence represented by SEQ ID NO: 76011.

The probes having the nucleotide sequence represented by SEQ ID NOS: 90248, 90249, 90250, 90251, 90252, 90253, 90254, 90255, 90256, 90257, 90258 were selected from the ORFs having the nucleotide sequence represented by SEQ ID NO: 76012.

The probes having the nucleotide sequence represented by SEQ ID NOS: 90259, 90260, 90261, 90262, 90263, 90264, 90265, 90266, 90267, 90268, 90269 were selected from the ORFs having the nucleotide sequence represented by SEQ ID NO: 76013.

The probes having the nucleotide sequence represented by SEQ ID NOS: 90270, 90271, 90272, 90273, 90274, 90275, 90276, 90277, 90278, 90279, 90280 were selected from the ORFs having the nucleotide sequence represented by SEQ ID NO: 76014.

The probes having the nucleotide sequence represented by SEQ ID NOS: 90281, 90282, 90283, 90284, 90285, 90286, 90287, 90288, 90289, 90290, 90291 were selected from the ORFs having the nucleotide sequence represented by SEQ ID NO: 76015.

The probes having the nucleotide sequence represented by SEQ ID NOS: 90292, 90293, 90294, 90295, 90296, 90297, 90298, 90299, 90300, 90301, 90302 were selected from the ORFs having the nucleotide sequence represented by SEQ ID NO: 76016.

The probes having the nucleotide sequence represented by SEQ ID NOS: 90303, 90304, 90305, 90306, 90307, 90308, 90309, 90310, 90311, 90312, 90313 were selected from the ORFs having the nucleotide sequence represented by SEQ ID NO: 76017.

The probes having the nucleotide sequence represented by SEQ ID NOS: 90314, 90315, 90316, 90317, 90318, 90319, 90320, 90321, 90322, 90323, 90324 were selected from the ORFs having the nucleotide sequence represented by SEQ ID NO: 76018.

The probes having the nucleotide sequence represented by SEQ ID NOS: 90325, 90326, 90327, 90328, 90329, 90330, 90331, 90332, 90333, 90334, 90335 were selected from the ORFs having the nucleotide sequence represented by SEQ ID NO: 76019.

The probes having the nucleotide sequence represented by SEQ ID NOS: 90336, 90337, 90338, 90339, 90340, 90341, 90342, 90343, 90344, 90345, 90346 were selected from the ORFs having the nucleotide sequence represented by SEQ ID NO: 76020.

The probes having the nucleotide sequence represented by SEQ ID NOS: 90347, 90348, 90349, 90350, 90351, 90352, 90353, 90354, 90355, 90356, 90357 were selected from the ORFs having the nucleotide sequence represented by SEQ ID NO: 76021.

The probes having the nucleotide sequence represented by SEQ ID NOS: 90358, 90359, 90360, 90361, 90362, 90363, 90364, 90365, 90366, 90367, 90368 were selected from the ORFs having the nucleotide sequence represented by SEQ ID NO: 76022.

The probes having the nucleotide sequence represented by SEQ ID NOS: 90369, 90370, 90371, 90372, 90373, 90374, 90375, 90376, 90377, 90378, 90379 were selected from the ORFs having the nucleotide sequence represented by SEQ ID NO: 76023.

The probes having the nucleotide sequence represented by SEQ ID NOS: 90380, 90381, 90382, 90383, 90384, 90385, 90386, 90387, 90388, 90389, 90390 were selected from the ORFs having the nucleotide sequence represented by SEQ ID NO: 76024.

The probes having the nucleotide sequence represented by SEQ ID NOS: 90391, 90392, 90393, 90394, 90395, 90396, 90397, 90398, 90399, 90400, 90401 were selected from the ORFs having the nucleotide sequence represented by SEQ ID NO: 76025.

The probes having the nucleotide sequence represented by SEQ ID NOS: 90402, 90403, 90404, 90405, 90406, 90407, 90408, 90409, 90410, 90411, 90412 were selected from the ORFs having the nucleotide sequence represented by SEQ ID NO: 76026.

The probes having the nucleotide sequence represented by SEQ ID NOS: 90413, 90414, 90415, 90416, 90417, 90418, 90419, 90420, 90421, 90422, 90423 were selected from the ORFs having the nucleotide sequence represented by SEQ ID NO: 76027.

The probes having the nucleotide sequence represented by SEQ ID NOS: 90424, 90425, 90426, 90427, 90428, 90429, 90430, 90431, 90432, 90433, 90434 were selected from the ORFs having the nucleotide sequence represented by SEQ ID NO: 76028.

The probes having the nucleotide sequence represented by SEQ ID NOS: 90435, 90436, 90437, 90438, 90439, 90440, 90441, 90442, 90443, 90444, 90445 were selected from the ORFs having the nucleotide sequence represented by SEQ ID NO: 76029.

The probes having the nucleotide sequence represented by SEQ ID NOS: 90446, 90447, 90448, 90449, 90450, 90451, 90452, 90453, 90454, 90455, 90456 were selected from the ORFs having the nucleotide sequence represented by SEQ ID NO: 76030.

The probes having the nucleotide sequence represented by SEQ ID NOS: 90457, 90458, 90459, 90460, 90461, 90462, 90463, 90464, 90465, 90466, 90467 were selected from the ORFs having the nucleotide sequence represented by SEQ ID NO: 76031.

The probes having the nucleotide sequence represented by SEQ ID NOS: 90468, 90469, 90470, 90471, 90472, 90473, 90474, 90475, 90476, 90477, 90478 were selected from the ORFs having the nucleotide sequence represented by SEQ ID NO: 76032.

The probes having the nucleotide sequence represented by SEQ ID NOS: 90479, 90480, 90481, 90482, 90483, 90484, 90485, 90486, 90487, 90488, 90489 were selected from the ORFs having the nucleotide sequence represented by SEQ ID NO: 76033.

The probes having the nucleotide sequence represented by SEQ ID NOS: 90490, 90491, 90492, 90493, 90494, 90495, 90496, 90497, 90498, 90499, 90500 were selected from the ORFs having the nucleotide sequence represented by SEQ ID NO: 76034.

The probes having the nucleotide sequence represented by SEQ ID NOS: 90501, 90502, 90503, 90504, 90505, 90506, 90507, 90508, 90509, 90510, 90511 were selected from the ORFs having the nucleotide sequence represented by SEQ ID NO: 76035.

The probes having the nucleotide sequence represented by SEQ ID NOS: 90512, 90513, 90514, 90515, 90516, 90517, 90518, 90519, 90520, 90521, 90522 were selected from the ORFs having the nucleotide sequence represented by SEQ ID NO: 76036.

The probes having the nucleotide sequence represented by SEQ ID NOS: 90523, 90524, 90525, 90526, 90527, 90528, 90529, 90530, 90531, 90532, 90533 were selected from the ORFs having the nucleotide sequence represented by SEQ ID NO: 76037.

The probes having the nucleotide sequence represented by SEQ ID NOS: 90534, 90535, 90536, 90537, 90538, 90539, 90540, 90541, 90542, 90543, 90544 were selected from the ORFs having the nucleotide sequence represented by SEQ ID NO: 76038.

The probes having the nucleotide sequence represented by SEQ ID NOS: 90545, 90546, 90547, 90548, 90549, 90550, 90551, 90552, 90553, 90554, 90555 were selected from the ORFs having the nucleotide sequence represented by SEQ ID NO: 76039.

The probes having the nucleotide sequence represented by SEQ ID NOS: 90556, 90557, 90558, 90559, 90560, 90561, 90562, 90563, 90564, 90565, 90566 were selected from the ORFs having the nucleotide sequence represented by SEQ ID NO: 76040.

The probes having the nucleotide sequence represented by SEQ ID NOS: 90567, 90568, 90569, 90570, 90571, 90572, 90573, 90574, 90575, 90576, 90577 were selected from the ORFs having the nucleotide sequence represented by SEQ ID NO: 76041.

The probes having the nucleotide sequence represented by SEQ ID NOS: 90578, 90579, 90580, 90581, 90582, 90583, 90584, 90585, 90586, 90587, 90588 were selected from the ORFs having the nucleotide sequence represented by SEQ ID NO: 76042.

The probes having the nucleotide sequence represented by SEQ ID NOS: 90589, 90590, 90591, 90592, 90593, 90594, 90595, 90596, 90597, 90598, 90599 were selected from the ORFs having the nucleotide sequence represented by SEQ ID NO: 76043.

The probes having the nucleotide sequence represented by SEQ ID NOS: 90600, 90601, 90602, 90603, 90604, 90605, 90606, 90607, 90608, 90609, 90610 were selected from the ORFs having the nucleotide sequence represented by SEQ ID NO: 76044.

The probes having the nucleotide sequence represented by SEQ ID NOS: 90611, 90612, 90613, 90614, 90615, 90616, 90617, 90618, 90619, 90620, 90621 were selected from the ORFs having the nucleotide sequence represented by SEQ ID NO: 76045.

The probes having the nucleotide sequence represented by SEQ ID NOS: 90622, 90623, 90624, 90625, 90626, 90627, 90628, 90629, 90630, 90631, 90632 were selected from the ORFs having the nucleotide sequence represented by SEQ ID NO: 76046.

The probes having the nucleotide sequence represented by SEQ ID NOS: 90633, 90634, 90635, 90636, 90637, 90638, 90639, 90640, 90641, 90642, 90643 were selected from the ORFs having the nucleotide sequence represented by SEQ ID NO: 76047.

The probes having the nucleotide sequence represented by SEQ ID NOS: 90644, 90645, 90646, 90647, 90648, 90649, 90650, 90651, 90652, 90653, 90654 were selected from the ORFs having the nucleotide sequence represented by SEQ ID NO: 76048.

The probes having the nucleotide sequence represented by SEQ ID NOS: 90655, 90656, 90657, 90658, 90659, 90660, 90661, 90662, 90663, 90664, 90665 were selected from the ORFs having the nucleotide sequence represented by SEQ ID NO: 76049.

The probes having the nucleotide sequence represented by SEQ ID NOS: 90666, 90667, 90668, 90669, 90670, 90671,

90672, 90673, 90674, 90675, 90676 were selected from the ORFs having the nucleotide sequence represented by SEQ ID NO: 76050.

The probes having the nucleotide sequence represented by SEQ ID NOS: 90677, 90678, 90679, 90680, 90681, 90682, 90683, 90684, 90685, 90686, 90687 were selected from the ORFs having the nucleotide sequence represented by SEQ ID NO: 76051.

The probes having the nucleotide sequence represented by SEQ ID NOS: 90688, 90689, 90690, 90691, 90692, 90693, 90694, 90695, 90696, 90697, 90698 were selected from the ORFs having the nucleotide sequence represented by SEQ ID NO: 76052.

The probes having the nucleotide sequence represented by SEQ ID NOS: 90699, 90700, 90701, 90702, 90703, 90704, 90705, 90706, 90707, 90708, 90709 were selected from the ORFs having the nucleotide sequence represented by SEQ ID NO: 76053.

The probes having the nucleotide sequence represented by SEQ ID NOS: 90710, 90711, 90712, 90713, 90714, 90715, 90716, 90717, 90718, 90719, 90720 were selected from the ORFs having the nucleotide sequence represented by SEQ ID NO: 76054.

The probes having the nucleotide sequence represented by SEQ ID NOS: 90721, 90722, 90723, 90724, 90725, 90726, 90727, 90728, 90729, 90730, 90731 were selected from the ORFs having the nucleotide sequence represented by SEQ ID NO: 76055.

The probes having the nucleotide sequence represented by SEQ ID NOS: 90732, 90733, 90734, 90735, 90736, 90737, 90738, 90739, 90740, 90741, 90742 were selected from the ORFs having the nucleotide sequence represented by SEQ ID NO: 76056.

The probes having the nucleotide sequence represented by SEQ ID NOS: 90743, 90744, 90745, 90746, 90747, 90748, 90749, 90750, 90751, 90752, 90753 were selected from the ORFs having the nucleotide sequence represented by SEQ ID NO: 76057.

The probes having the nucleotide sequence represented by SEQ ID NOS: 90754, 90755, 90756, 90757, 90758, 90759, 90760, 90761, 90762, 90763, 90764 were selected from the ORFs having the nucleotide sequence represented by SEQ ID NO: 76058.

The probes having the nucleotide sequence represented by SEQ ID NOS: 90765, 90766, 90767, 90768, 90769, 90770, 90771, 90772, 90773, 90774, 90775 were selected from the ORFs having the nucleotide sequence represented by SEQ ID NO: 76059.

The probes having the nucleotide sequence represented by SEQ ID NOS: 90776, 90777, 90778, 90779, 90780, 90781, 90782, 90783, 90784, 90785, 90786 were selected from the ORFs having the nucleotide sequence represented by SEQ ID NO: 76060.

The probes having the nucleotide sequence represented by SEQ ID NOS: 90787, 90788, 90789, 90790, 90791, 90792, 90793, 90794, 90795, 90796, 90797 were selected from the ORFs having the nucleotide sequence represented by SEQ ID NO: 76061.

The probes having the nucleotide sequence represented by SEQ ID NOS: 90798, 90799, 90800, 90801, 90802, 90803, 90804, 90805, 90806, 90807, 90808 were selected from the ORFs having the nucleotide sequence represented by SEQ ID NO: 76062.

The probes having the nucleotide sequence represented by SEQ ID NOS: 90809, 90810, 90811, 90812, 90813, 90814, 90815, 90816, 90817, 90818, 90819 were selected from the ORFs having the nucleotide sequence represented by SEQ ID NO: 76063.

The probes having the nucleotide sequence represented by SEQ ID NOS: 90820, 90821, 90822, 90823, 90824, 90825, 90826, 90827, 90828, 90829, 90830 were selected from the ORFs having the nucleotide sequence represented by SEQ ID NO: 76064.

The probes having the nucleotide sequence represented by SEQ ID NOS: 90831, 90832, 90833, 90834, 90835, 90836, 90837, 90838, 90839, 90840, 90841 were selected from the ORFs having the nucleotide sequence represented by SEQ ID NO: 76065.

The probes having the nucleotide sequence represented by SEQ ID NOS: 90842, 90843, 90844, 90845, 90846, 90847, 90848, 90849, 90850, 90851, 90852 were selected from the ORFs having the nucleotide sequence represented by SEQ ID NO: 76066.

The probes having the nucleotide sequence represented by SEQ ID NOS: 90853, 90854, 90855, 90856, 90857, 90858, 90859, 90860, 90861, 90862, 90863 were selected from the ORFs having the nucleotide sequence represented by SEQ ID NO: 76067.

The probes having the nucleotide sequence represented by SEQ ID NOS: 90864, 90865, 90866, 90867, 90868, 90869, 90870, 90871, 90872, 90873, 90874 were selected from the ORFs having the nucleotide sequence represented by SEQ ID NO: 76068.

The probes having the nucleotide sequence represented by SEQ ID NOS: 90875, 90876, 90877, 90878, 90879, 90880, 90881, 90882, 90883, 90884, 90885 were selected from the ORFs having the nucleotide sequence represented by SEQ ID NO: 76069.

The probes having the nucleotide sequence represented by SEQ ID NOS: 90886, 90887, 90888, 90889, 90890, 90891, 90892, 90893, 90894, 90895, 90896 were selected from the ORFs having the nucleotide sequence represented by SEQ ID NO: 76070.

The probes having the nucleotide sequence represented by SEQ ID NOS: 90897, 90898, 90899, 90900, 90901, 90902, 90903, 90904, 90905, 90906, 90907 were selected from the ORFs having the nucleotide sequence represented by SEQ ID NO: 76071.

The probes having the nucleotide sequence represented by SEQ ID NOS: 90908, 90909, 90910, 90911, 90912, 90913, 90914, 90915, 90916, 90917, 90918 were selected from the ORFs having the nucleotide sequence represented by SEQ ID NO: 76072.

The probes having the nucleotide sequence represented by SEQ ID NOS: 90919, 90920, 90921, 90922, 90923, 90924, 90925, 90926, 90927, 90928, 90929 were selected from the ORFs having the nucleotide sequence represented by SEQ ID NO: 76073.

The probes having the nucleotide sequence represented by SEQ ID NOS: 90930, 90931, 90932, 90933, 90934, 90935, 90936, 90937, 90938, 90939, 90940 were selected from the ORFs having the nucleotide sequence represented by SEQ ID NO: 76074.

The probes having the nucleotide sequence represented by SEQ ID NOS: 90941, 90942, 90943, 90944, 90945, 90946, 90947, 90948, 90949, 90950, 90951 were selected from the ORFs having the nucleotide sequence represented by SEQ ID NO: 76075.

The probes having the nucleotide sequence represented by SEQ ID NOS: 90952, 90953, 90954, 90955, 90956, 90957, 90958, 90959, 90960, 90961, 90962 were selected from the ORFs having the nucleotide sequence represented by SEQ ID NO: 76076.

The probes having the nucleotide sequence represented by SEQ ID NOS: 90963, 90964, 90965, 90966, 90967, 90968, 90969, 90970, 90971, 90972, 90973 were selected from the ORFs having the nucleotide sequence represented by SEQ ID NO: 76077.

The probes having the nucleotide sequence represented by SEQ ID NOS: 90974, 90975, 90976, 90977, 90978, 90979, 90980, 90981, 90982, 90983, 90984 were selected from the ORFs having the nucleotide sequence represented by SEQ ID NO: 76078.

The probes having the nucleotide sequence represented by SEQ ID NOS: 90985, 90986, 90987, 90988, 90989, 90990, 90991, 90992, 90993, 90994, 90995 were selected from the ORFs having the nucleotide sequence represented by SEQ ID NO: 76079.

The probes having the nucleotide sequence represented by SEQ ID NOS: 90996, 90997, 90998, 90999, 91000, 91001, 91002, 91003, 91004, 91005, 91006 were selected from the ORFs having the nucleotide sequence represented by SEQ ID NO: 76080.

The probes having the nucleotide sequence represented by SEQ ID NOS: 91007, 91008, 91009, 91010, 91011, 91012, 91013, 91014, 91015, 91016, 91017 were selected from the ORFs having the nucleotide sequence represented by SEQ ID NO: 76081.

The probes having the nucleotide sequence represented by SEQ ID NOS: 91018, 91019, 91020, 91021, 91022, 91023, 91024, 91025, 91026, 91027, 91028 were selected from the ORFs having the nucleotide sequence represented by SEQ ID NO: 76082.

The probes having the nucleotide sequence represented by SEQ ID NOS: 91029, 91030, 91031, 91032, 91033, 91034, 91035, 91036, 91037, 91038, 91039 were selected from the ORFs having the nucleotide sequence represented by SEQ ID NO: 76083.

The probes having the nucleotide sequence represented by SEQ ID NOS: 91040, 91041, 91042, 91043, 91044, 91045, 91046, 91047, 91048, 91049, 91050 were selected from the ORFs having the nucleotide sequence represented by SEQ ID NO: 76084.

The probes having the nucleotide sequence represented by SEQ ID NOS: 91051, 91052, 91053, 91054, 91055, 91056, 91057, 91058, 91059, 91060, 91061 were selected from the ORFs having the nucleotide sequence represented by SEQ ID NO: 76085.

The probes having the nucleotide sequence represented by SEQ ID NOS: 91062, 91063, 91064, 91065, 91066, 91067, 91068, 91069, 91070, 91071, 91072 were selected from the ORFs having the nucleotide sequence represented by SEQ ID NO: 76086.

The probes having the nucleotide sequence represented by SEQ ID NOS: 91073, 91074, 91075, 91076, 91077, 91078, 91079, 91080, 91081, 91082, 91083 were selected from the ORFs having the nucleotide sequence represented by SEQ ID NO: 76087.

The probes having the nucleotide sequence represented by SEQ ID NOS: 91084, 91085, 91086, 91087, 91088, 91089, 91090, 91091, 91092, 91093, 91094 were selected from the ORFs having the nucleotide sequence represented by SEQ ID NO: 76088.

The probes having the nucleotide sequence represented by SEQ ID NOS: 91095, 91096, 91097, 91098, 91099, 91100, 91101, 91102, 91103, 91104, 91105 were selected from the ORFs having the nucleotide sequence represented by SEQ ID NO: 76089.

The probes having the nucleotide sequence represented by SEQ ID NOS: 91106, 91107, 91108, 91109, 91110, 91111, 91112, 91113, 91114, 91115, 91116 were selected from the ORFs having the nucleotide sequence represented by SEQ ID NO: 76090.

The probes having the nucleotide sequence represented by SEQ ID NOS: 91117, 91118, 91119, 91120, 91121, 91122, 91123, 91124, 91125, 91126, 91127 were selected from the ORFs having the nucleotide sequence represented by SEQ ID NO: 76091.

The probes having the nucleotide sequence represented by SEQ ID NOS: 91128, 91129, 91130, 91131, 91132, 91133, 91134, 91135, 91136, 91137, 91138 were selected from the ORFs having the nucleotide sequence represented by SEQ ID NO: 76092.

The probes having the nucleotide sequence represented by SEQ ID NOS: 91139, 91140, 91141, 91142, 91143, 91144, 91145, 91146, 91147, 91148, 91149 were selected from the ORFs having the nucleotide sequence represented by SEQ ID NO: 76093.

The probes having the nucleotide sequence represented by SEQ ID NOS: 91150, 91151, 91152, 91153, 91154, 91155, 91156, 91157, 91158, 91159, 91160 were selected from the ORFs having the nucleotide sequence represented by SEQ ID NO: 76094.

The probes having the nucleotide sequence represented by SEQ ID NOS: 91161, 91162, 91163, 91164, 91165, 91166, 91167, 91168, 91169, 91170, 91171 were selected from the ORFs having the nucleotide sequence represented by SEQ ID NO: 76095.

The probes having the nucleotide sequence represented by SEQ ID NOS: 91172, 91173, 91174, 91175, 91176, 91177, 91178, 91179, 91180, 91181, 91182 were selected from the ORFs having the nucleotide sequence represented by SEQ ID NO: 76096.

The probes having the nucleotide sequence represented by SEQ ID NOS: 91183, 91184, 91185, 91186, 91187, 91188, 91189, 91190, 91191, 91192, 91193 were selected from the ORFs having the nucleotide sequence represented by SEQ ID NO: 76097.

The probes having the nucleotide sequence represented by SEQ ID NOS: 91194, 91195, 91196, 91197, 91198, 91199, 91200, 91201, 91202, 91203, 91204 were selected from the ORFs having the nucleotide sequence represented by SEQ ID NO: 76098.

The probes having the nucleotide sequence represented by SEQ ID NOS: 91205, 91206, 91207, 91208, 91209, 91210, 91211, 91212, 91213, 91214, 91215 were selected from the ORFs having the nucleotide sequence represented by SEQ ID NO: 76099.

The probes having the nucleotide sequence represented by SEQ ID NOS: 91216, 91217, 91218, 91219, 91220, 91221, 91222, 91223, 91224, 91225, 91226 were selected from the ORFs having the nucleotide sequence represented by SEQ ID NO: 76100.

The probes having the nucleotide sequence represented by SEQ ID NOS: 91227, 91228, 91229, 91230, 91231, 91232, 91233, 91234, 91235, 91236, 91237 were selected from the ORFs having the nucleotide sequence represented by SEQ ID NO: 76101.

The probes having the nucleotide sequence represented by SEQ ID NOS: 91238, 91239, 91240, 91241, 91242, 91243, 91244, 91245, 91246, 91247, 91248, 91249, 91250, 91251, 91252, 91253, 91254, 91255, 91256, 91257, 91258, 91259 were selected from the ORFs having the nucleotide sequence represented by SEQ ID NO: 76102.

The probes having the nucleotide sequence represented by SEQ ID NOS: 91260, 91261, 91262, 91263, 91264, 91265, 91266, 91267, 91268, 91269, 91270 were selected from the ORFs having the nucleotide sequence represented by SEQ ID NO: 76103.

The probes having the nucleotide sequence represented by SEQ ID NOS: 91271, 91272, 91273, 91274, 91275, 91276, 91277, 91278, 91279, 91280, 91281 were selected from the ORFs having the nucleotide sequence represented by SEQ ID NO: 76104.

The probes having the nucleotide sequence represented by SEQ ID NOS: 91282, 91283, 91284, 91285, 91286, 91287, 91288, 91289, 91290, 91291, 91292 were selected from the ORFs having the nucleotide sequence represented by SEQ ID NO: 76105.

The probes having the nucleotide sequence represented by SEQ ID NOS: 91293, 91294, 91295, 91296, 91297, 91298, 91299, 91300, 91301, 91302, 91303 were selected from the ORFs having the nucleotide sequence represented by SEQ ID NO: 76106.

The probes having the nucleotide sequence represented by SEQ ID NOS: 91304, 91305, 91306, 91307, 91308, 91309, 91310, 91311, 91312, 91313, 91314 were selected from the ORFs having the nucleotide sequence represented by SEQ ID NO: 76107.

The probes having the nucleotide sequence represented by SEQ ID NOS: 91315, 91316, 91317, 91318, 91319, 91320, 91321, 91322, 91323, 91324, 91325 were selected from the ORFs having the nucleotide sequence represented by SEQ ID NO: 76108.

The probes having the nucleotide sequence represented by SEQ ID NOS: 91326, 91327, 91328, 91329, 91330, 91331, 91332, 91333, 91334, 91335, 91336 were selected from the ORFs having the nucleotide sequence represented by SEQ ID NO: 76109.

The probes having the nucleotide sequence represented by SEQ ID NOS: 91337, 91338, 91339, 91340, 91341, 91342, 91343, 91344, 91345, 91346, 91347 were selected from the ORFs having the nucleotide sequence represented by SEQ ID NO: 76110.

The probes having the nucleotide sequence represented by SEQ ID NOS: 91348, 91349, 91350, 91351, 91352, 91353, 91354, 91355, 91356, 91357, 91358 were selected from the ORFs having the nucleotide sequence represented by SEQ ID NO: 76111.

The probes having the nucleotide sequence represented by SEQ ID NOS: 91359, 91360, 91361, 91362, 91363, 91364, 91365, 91366, 91367, 91368, 91369 were selected from the ORFs having the nucleotide sequence represented by SEQ ID NO: 76112.

The probes having the nucleotide sequence represented by SEQ ID NOS: 91370, 91371, 91372, 91373, 91374, 91375, 91376, 91377, 91378, 91379, 91380 were selected from the ORFs having the nucleotide sequence represented by SEQ ID NO: 76113.

The probes having the nucleotide sequence represented by SEQ ID NOS: 91381, 91382, 91383, 91384, 91385, 91386, 91387, 91388, 91389, 91390, 91391 were selected from the ORFs having the nucleotide sequence represented by SEQ ID NO: 76114.

The probes having the nucleotide sequence represented by SEQ ID NOS: 91392, 91393, 91394, 91395, 91396, 91397, 91398, 91399, 91400, 91401, 91402 were selected from the ORFs having the nucleotide sequence represented by SEQ ID NO: 76115.

The probes having the nucleotide sequence represented by SEQ ID NOS: 91403, 91404, 91405, 91406, 91407, 91408, 91409, 91410, 91411, 91412, 91413 were selected from the ORFs having the nucleotide sequence represented by SEQ ID NO: 76116.

The probes having the nucleotide sequence represented by SEQ ID NOS: 91414, 91415, 91416, 91417, 91418, 91419, 91420, 91421, 91422, 91423, 91424 were selected from the ORFs having the nucleotide sequence represented by SEQ ID NO: 76117.

The probes having the nucleotide sequence represented by SEQ ID NOS: 91425, 91426, 91427, 91428, 91429, 91430, 91431, 91432, 91433, 91434, 91435 were selected from the ORFs having the nucleotide sequence represented by SEQ ID NO: 76118.

The probes having the nucleotide sequence represented by SEQ ID NOS: 91436, 91437, 91438, 91439, 91440, 91441, 91442, 91443, 91444, 91445, 91446 were selected from the ORFs having the nucleotide sequence represented by SEQ ID NO: 76119.

The probes having the nucleotide sequence represented by SEQ ID NOS: 91447, 91448, 91449, 91450, 91451, 91452, 91453, 91454, 91455, 91456, 91457 were selected from the ORFs having the nucleotide sequence represented by SEQ ID NO: 76120.

The probes having the nucleotide sequence represented by SEQ ID NOS: 91458, 91459, 91460, 91461, 91462, 91463, 91464, 91465, 91466, 91467, 91468 were selected from the ORFs having the nucleotide sequence represented by SEQ ID NO: 76121.

The probes having the nucleotide sequence represented by SEQ ID NOS: 91469, 91470, 91471, 91472, 91473, 91474, 91475, 91476, 91477, 91478, 91479 were selected from the ORFs having the nucleotide sequence represented by SEQ ID NO: 76122.

The probes having the nucleotide sequence represented by SEQ ID NOS: 91480, 91481, 91482, 91483, 91484, 91485, 91486, 91487, 91488, 91489, 91490 were selected from the ORFs having the nucleotide sequence represented by SEQ ID NO: 76123.

The probes having the nucleotide sequence represented by SEQ ID NOS: 91491, 91492, 91493, 91494, 91495, 91496, 91497, 91498, 91499, 91500, 91501 were selected from the ORFs having the nucleotide sequence represented by SEQ ID NO: 76124.

The probes having the nucleotide sequence represented by SEQ ID NOS: 91502, 91503, 91504, 91505, 91506, 91507, 91508, 91509, 91510, 91511, 91512 were selected from the ORFs having the nucleotide sequence represented by SEQ ID NO: 76125.

The probes having the nucleotide sequence represented by SEQ ID NOS: 91513, 91514, 91515, 91516, 91517, 91518, 91519, 91520, 91521, 91522, 91523 were selected from the ORFs having the nucleotide sequence represented by SEQ ID NO: 76126.

The probes having the nucleotide sequence represented by SEQ ID NOS: 91524, 91525, 91526, 91527, 91528, 91529, 91530, 91531, 91532, 91533, 91534, 91535, 91536, 91537, 91538, 91539, 91540, 91541, 91542, 91543, 91544, 91545 were selected from the ORFs having the nucleotide sequence represented by SEQ ID NO: 76127.

The probes having the nucleotide sequence represented by SEQ ID NOS: 91546, 91547, 91548, 91549, 91550, 91551, 91552, 91553, 91554, 91555, 91556 were selected from the ORFs having the nucleotide sequence represented by SEQ ID NO: 76128.

The probes having the nucleotide sequence represented by SEQ ID NOS: 91557, 91558, 91559, 91560, 91561, 91562, 91563, 91564, 91565, 91566, 91567 were selected from the ORFs having the nucleotide sequence represented by SEQ ID NO: 76129.

The probes having the nucleotide sequence represented by SEQ ID NOS: 91568, 91569, 91570, 91571, 91572, 91573, 91574, 91575, 91576, 91577, 91578 were selected from the ORFs having the nucleotide sequence represented by SEQ ID NO: 76130.

The probes having the nucleotide sequence represented by SEQ ID NOS: 91579, 91580, 91581, 91582, 91583, 91584, 91585, 91586, 91587, 91588, 91589 were selected from the ORFs having the nucleotide sequence represented by SEQ ID NO: 76131.

The probes having the nucleotide sequence represented by SEQ ID NOS: 91590, 91591, 91592, 91593, 91594, 91595, 91596, 91597, 91598, 91599, 91600 were selected from the ORFs having the nucleotide sequence represented by SEQ ID NO: 76132.

The probes having the nucleotide sequence represented by SEQ ID NOS: 91601, 91602, 91603, 91604, 91605, 91606, 91607, 91608, 91609, 91610, 91611 were selected from the ORFs having the nucleotide sequence represented by SEQ ID NO: 76133.

The probes having the nucleotide sequence represented by SEQ ID NOS: 91612, 91613, 91614, 91615, 91616, 91617, 91618, 91619, 91620, 91621, 91622 were selected from the ORFs having the nucleotide sequence represented by SEQ ID NO: 76134.

The probes having the nucleotide sequence represented by SEQ ID NOS: 91623, 91624, 91625, 91626, 91627, 91628, 91629, 91630, 91631, 91632, 91633 were selected from the ORFs having the nucleotide sequence represented by SEQ ID NO: 76135.

The probes having the nucleotide sequence represented by SEQ ID NOS: 91634, 91635, 91636, 91637, 91638, 91639, 91640, 91641, 91642, 91643, 91644 were selected from the ORFs having the nucleotide sequence represented by SEQ ID NO: 76136.

The probes having the nucleotide sequence represented by SEQ ID NOS: 91645, 91646, 91647, 91648, 91649, 91650, 91651, 91652, 91653, 91654, 91655 were selected from the ORFs having the nucleotide sequence represented by SEQ ID NO: 76137.

The probes having the nucleotide sequence represented by SEQ ID NOS: 91656, 91657, 91658, 91659, 91660, 91661, 91662, 91663, 91664, 91665, 91666 were selected from the ORFs having the nucleotide sequence represented by SEQ ID NO: 76138.

The probes having the nucleotide sequence represented by SEQ ID NOS: 91667, 91668, 91669, 91670, 91671, 91672, 91673, 91674, 91675, 91676, 91677 were selected from the ORFs having the nucleotide sequence represented by SEQ ID NO: 76139.

The probes having the nucleotide sequence represented by SEQ ID NOS: 91678, 91679, 91680, 91681, 91682, 91683, 91684, 91685, 91686, 91687, 91688 were selected from the ORFs having the nucleotide sequence represented by SEQ ID NO: 76140.

The probes having the nucleotide sequence represented by SEQ ID NOS: 91689, 91690, 91691, 91692, 91693, 91694, 91695, 91696, 91697, 91698, 91699 were selected from the ORFs having the nucleotide sequence represented by SEQ ID NO: 76141.

The probes having the nucleotide sequence represented by SEQ ID NOS: 91700, 91701, 91702, 91703, 91704, 91705, 91706, 91707, 91708, 91709, 91710 were selected from the ORFs having the nucleotide sequence represented by SEQ ID NO: 76142.

The probes having the nucleotide sequence represented by SEQ ID NOS: 91711, 91712, 91713, 91714, 91715, 91716, 91717, 91718, 91719, 91720, 91721 were selected from the ORFs having the nucleotide sequence represented by SEQ ID NO: 76143.

The probes having the nucleotide sequence represented by SEQ ID NOS: 91722, 91723, 91724, 91725, 91726, 91727, 91728, 91729, 91730, 91731, 91732 were selected from the ORFs having the nucleotide sequence represented by SEQ ID NO: 76144.

The probes having the nucleotide sequence represented by SEQ ID NOS: 91733, 91734, 91735, 91736, 91737, 91738, 91739, 91740, 91741, 91742, 91743 were selected from the ORFs having the nucleotide sequence represented by SEQ ID NO: 76145.

The probes having the nucleotide sequence represented by SEQ ID NOS: 91744, 91745, 91746, 91747, 91748, 91749, 91750, 91751, 91752, 91753, 91754 were selected from the ORFs having the nucleotide sequence represented by SEQ ID NO: 76146.

The probes having the nucleotide sequence represented by SEQ ID NOS: 91755, 91756, 91757, 91758, 91759, 91760, 91761, 91762, 91763, 91764, 91765 were selected from the ORFs having the nucleotide sequence represented by SEQ ID NO: 76147.

The probes having the nucleotide sequence represented by SEQ ID NOS: 91766, 91767, 91768, 91769, 91770, 91771, 9177.2, 91773, 91774, 91775, 91776 were selected from the ORFs having the nucleotide sequence represented by SEQ ID NO: 76148.

The probes having the nucleotide sequence represented by SEQ ID NOS: 91777, 91778, 91779, 91780, 91781, 91782, 91783, 91784, 91785, 91786, 91787 were selected from the ORFs having the nucleotide sequence represented by SEQ ID NO: 76149.

The probes having the nucleotide sequence represented by SEQ ID NOS: 91788, 91789, 91790, 91791, 91792, 91793, 91794, 91795, 91796, 91797, 91798 were selected from the ORFs having the nucleotide sequence represented by SEQ ID NO: 76150.

The probes having the nucleotide sequence represented by SEQ ID NOS: 91799, 91800, 91801, 91802, 91803, 91804, 91805, 91806, 91807, 91808, 91809 were selected from the ORFs having the nucleotide sequence represented by SEQ ID NO: 76151.

The probes having the nucleotide sequence represented by SEQ ID NOS: 91810, 91811, 91812, 91813, 91814, 91815, 91816, 91817, 91818, 91819, 91820 were selected from the ORFs having the nucleotide sequence represented by SEQ ID NO: 76152.

The probes having the nucleotide sequence represented by SEQ ID NOS: 91821, 91822, 91823, 91824, 91825, 91826, 91827, 91828, 91829, 91830, 91831 were selected from the ORFs having the nucleotide sequence represented by SEQ ID NO: 76153.

The probes having the nucleotide sequence represented by SEQ ID NOS: 91832, 91833, 91834, 91835, 91836, 91837, 91838, 91839, 91840, 91841, 91842 were selected from the ORFs having the nucleotide sequence represented by SEQ ID NO: 76154.

The probes having the nucleotide sequence represented by SEQ ID NOS: 91843, 91844, 91845, 91846, 91847, 91848, 91849, 91850, 91851, 91852, 91853 were selected from the ORFs having the nucleotide sequence represented by SEQ ID NO: 76155.

The probes having the nucleotide sequence represented by SEQ ID NOS: 91854, 91855, 91856, 91857, 91858, 91859, 91860, 91861, 91862, 91863, 91864 were selected from the ORFs having the nucleotide sequence represented by SEQ ID NO: 76156.

The probes having the nucleotide sequence represented by SEQ ID NOS: 91865, 91866, 91867, 91868, 91869, 91870, 91871, 91872, 91873, 91874, 91875 were selected from the ORFs having the nucleotide sequence represented by SEQ ID NO: 76157.

The probes having the nucleotide sequence represented by SEQ ID NOS: 91876, 91877, 91878, 91879, 91880, 91881, 91882, 91883, 91884, 91885, 91886 were selected from the ORFs having the nucleotide sequence represented by SEQ ID NO: 76158.

The probes having the nucleotide sequence represented by SEQ ID NOS: 91887, 91888, 91889, 91890, 91891, 91892, 91893, 91894, 91895, 91896, 91897 were selected from the ORFs having the nucleotide sequence represented by SEQ ID NO: 76159.

The probes having the nucleotide sequence represented by SEQ ID NOS: 91898, 91899, 91900, 91901, 91902, 91903, 91904, 91905, 91906, 91907, 91908 were selected from the ORFs having the nucleotide sequence represented by SEQ ID NO: 76160.

The probes having the nucleotide sequence represented by SEQ ID NOS: 91909, 91910, 91911, 91912, 91913, 91914, 91915, 91916, 91917, 91918, 91919 were selected from the ORFs having the nucleotide sequence represented by SEQ ID NO: 76161.

The probes having the nucleotide sequence represented by SEQ ID NOS: 91920, 91921, 91922, 91923, 91924, 91925, 91926, 91927, 91928, 91929, 91930 were selected from the ORFs having the nucleotide sequence represented by SEQ ID NO: 76162.

The probes having the nucleotide sequence represented by SEQ ID NOS: 91931, 91932, 91933, 91934, 91935, 91936, 91937, 91938,. 91939, 91940, 91941 were selected from the ORFs having the nucleotide sequence represented by SEQ ID NO: 76163.

The probes having the nucleotide sequence represented by SEQ ID NOS: 91942, 91943, 91944, 91945, 91946, 91947, 91948, 91949, 91950, 91951, 91952 were selected from the ORFs having the nucleotide sequence represented by SEQ ID NO: 76164.

The probes having the nucleotide sequence represented by SEQ ID NOS: 91953, 91954, 91955, 91956, 91957, 91958, 91959, 91960, 91961, 91962, 91963 were selected from the ORFs having the nucleotide sequence represented by SEQ ID NO: 76165.

The probes having the nucleotide sequence represented by SEQ ID NOS: 91964, 91965, 91966, 91967, 91968, 91969, 91970, 91971, 91972, 91973, 91974 were selected from the ORFs having the nucleotide sequence represented by SEQ ID NO: 76166.

The probes having the nucleotide sequence represented by SEQ ID NOS: 91975, 91976, 91977, 91978, 91979, 91980, 91981, 91982, 91983, 91984, 91985 were selected from the ORFs having the nucleotide sequence represented by SEQ ID NO: 76167.

The probes having the nucleotide sequence represented by SEQ ID NOS: 91986, 91987, 91988, 91989, 91990, 91991, 91992, 91993, 91994, 91995, 91996 were selected from the ORFs having the nucleotide sequence represented by SEQ ID NO: 76168.

The probes having the nucleotide sequence represented by SEQ ID NOS: 91997, 91998, 91999, 92000, 92001, 92002, 92003, 92004, 92005, 92006, 92007 were selected from the ORFs having the nucleotide sequence represented by SEQ ID NO: 76169.

The probes having the nucleotide sequence represented by SEQ ID NOS: 92008, 92009, 92010, 92011, 92012, 92013, 92014, 92015, 92016, 92017, 92018 were selected from the ORFs having the nucleotide sequence represented by SEQ ID NO: 76170.

The probes having the nucleotide sequence represented by SEQ ID NOS: 92019, 92020, 92021, 92022, 92023, 92024, 92025, 92026, 92027, 92028, 92029 were selected from the ORFs having the nucleotide sequence represented by SEQ ID NO: 76171.

The probes having the nucleotide sequence represented by SEQ ID NOS: 92030, 92031, 92032, 92033, 92034, 92035, 92036, 92037, 92038, 92039, 92040 were selected from the ORFs having the nucleotide sequence represented by SEQ ID NO: 76172.

The probes having the nucleotide sequence represented by SEQ ID NOS: 92041, 92042, 92043, 92044, 92045, 92046, 92047, 92048, 92049, 92050, 92051 were selected from the ORFs having the nucleotide sequence represented by SEQ ID NO: 76173.

The probes having the nucleotide sequence represented by SEQ ID NOS: 92052, 92053, 92054, 92055, 92056, 92057, 92058, 92059, 92060, 92061, 92062 were selected from the ORFs having the nucleotide sequence represented by SEQ ID NO: 76174.

The probes having the nucleotide sequence represented by SEQ ID NOS: 92063, 92064, 92065, 92066, 92067, 92068, 92069, 92070, 92071, 92072, 92073 were selected from the ORFs having the nucleotide sequence represented by SEQ ID NO: 76175.

The probes having the nucleotide sequence represented by SEQ ID NOS: 92074, 92075, 92076, 92077, 92078, 92079, 92080, 92081, 92082, 92083, 92084 were selected from the ORFs having the nucleotide sequence represented by SEQ ID NO: 76176.

The probes having the nucleotide sequence represented by SEQ ID NOS: 92085, 92086, 92087, 92088, 92089, 92090, 92091, 92092, 92093, 92094, 92095 were selected from the ORFs having the nucleotide sequence represented by SEQ ID NO: 76177.

The probes having the nucleotide sequence represented by SEQ ID NOS.: 92096, 92097, 92098, 92099, 92100, 92101, 92102, 92103, 92104, 92105, 92106 were selected from the ORFs having the nucleotide sequence represented by SEQ ID NO: 76178.

The probes having the nucleotide sequence represented by SEQ ID NOS: 92107, 92108, 92109, 92110, 92111, 92112, 92113, 92114, 92115, 92116, 92117 were selected from the ORFs having the nucleotide sequence represented by SEQ ID NO: 76179.

The probes having the nucleotide sequence represented by SEQ ID NOS: 92118, 92119, 92120, 92121, 92122, 92123, 92124, 92125, 92126, 92127, 92128 were selected from the ORFs having the nucleotide sequence represented by SEQ ID NO: 76180.

The probes having the nucleotide sequence represented by SEQ ID NOS: 92129, 92130, 92131, 92132, 92133, 92134, 92135, 92136, 92137, 92138, 92139 were selected from the ORFs having the nucleotide sequence represented by SEQ ID NO: 76181.

The probes having the nucleotide sequence represented by SEQ ID NOS: 92140, 92141, 92142, 92143, 92144, 92145, 92146, 92147, 92148, 92149, 92150, 92151, 92152, 92153, 92154, 92155, 92156, 92157, 92158, 92159, 92160, 92161 were selected from the ORFs having the nucleotide sequence represented by SEQ ID NO: 76182.

The probes having the nucleotide sequence represented by SEQ ID NOS: 92162, 92163, 92164, 92165, 92166, 92167, 92168, 92169, 92170, 92171, 92172 were selected from the ORFs having the nucleotide sequence represented by SEQ ID NO: 76183.

The probes having the nucleotide sequence represented by SEQ ID NOS: 92173, 92174, 92175, 92176, 92177, 92178, 92179, 92180, 92181, 92182, 92183 were selected from the ORFs having the nucleotide sequence represented by SEQ ID NO: 76184.

The probes having the nucleotide sequence represented by SEQ ID NOS: 92184, 92185, 92186, 92187, 92188, 92189, 92190, 92191, 92192, 92193, 92194 were selected from the ORFs having the nucleotide sequence represented by SEQ ID NO: 76185.

The probes having the nucleotide sequence represented by SEQ ID NOS: 92195, 92196, 92197, 92198, 92199, 92200, 92201, 92202, 92203, 92204, 92205 were selected from the ORFs having the nucleotide sequence represented by SEQ ID NO: 76186.

The probes having the nucleotide sequence represented by SEQ ID NOS: 92206, 92207, 92208, 92209, 92210, 92211, 92212, 92213, 92214, 92215, 92216 were selected from the ORFs having the nucleotide sequence represented by SEQ ID NO: 76187.

The probes having the nucleotide sequence represented by SEQ ID NOS: 92217, 92218, 92219, 92220, 92221, 92222, 92223, 92224, 92225, 92226, 92227 were selected from the ORFs having the nucleotide sequence represented by SEQ ID NO: 76188.

The probes having the nucleotide sequence represented by SEQ ID NOS: 92228, 92229, 92230, 92231, 92232, 92233, 92234, 92235, 92236, 92237, 92238 were selected from the ORFs having the nucleotide sequence represented by SEQ ID NO: 76189.

The probes having the nucleotide sequence represented by SEQ ID NOS: 92239, 92240, 92241, 92242, 92243, 92244, 92245, 92246, 92247, 92248, 92249 were selected from the ORFs having the nucleotide sequence represented by SEQ ID NO: 76190.

The probes having the nucleotide sequence represented by SEQ ID NOS: 92250, 92251, 92252, 92253, 92254, 92255, 92256, 92257, 92258, 92259, 92260 were selected from the ORFs having the nucleotide sequence represented by SEQ ID NO: 76191.

The probes having the nucleotide sequence represented by SEQ ID NOS: 92261, 92262, 92263, 92264, 92265, 92266, 92267, 92268, 92269, 92270, 92271 were selected from the ORFs having the nucleotide sequence represented by SEQ ID NO: 76192.

The probes having the nucleotide sequence represented by SEQ ID NOS: 92272, 92273, 92274, 92275, 92276, 92277, 92278, 92279, 92280, 92281, 92282 were selected from the ORFs having the nucleotide sequence represented by SEQ ID NO: 76193.

The probes having the nucleotide sequence represented by SEQ ID NOS: 92283, 92284, 92285, 92286, 92287, 92288, 92289, 92290, 92291, 92292, 92293 were selected from the ORFs having the nucleotide sequence represented by SEQ ID NO: 76194.

The probes having the nucleotide sequence represented by SEQ ID NOS: 92294, 92295, 92296, 92297, 92298, 92299, 92300, 92301, 92302, 92303, 92304 were selected from the ORFs having the nucleotide sequence represented by SEQ ID NO: 76195.

The probes having the nucleotide sequence represented by SEQ ID NOS: 92305, 92306, 92307, 92308, 92309, 92310, 92311, 92312, 92313, 92314, 92315 were selected from the ORFs having the nucleotide sequence represented by SEQ ID NO: 76196.

The probes having the nucleotide sequence represented by SEQ ID NOS: 92316, 92317, 92318, 92319, 92320, 92321, 92322, 92323, 92324, 92325, 92326 were selected from the ORFs having the nucleotide sequence represented by SEQ ID NO: 76197.

The probes having the nucleotide sequence represented by SEQ ID NOS: 92327, 92328, 92329, 92330, 92331, 92332, 92333, 92334, 92335, 92336, 92337 were selected from the ORFs having the nucleotide sequence represented by SEQ ID NO: 76198.

The probes having the nucleotide sequence represented by SEQ ID NOS: 92338, 92339, 92340, 92341, 92342, 92343, 92344, 92345, 92346, 92347, 92348 were selected from the ORFs having the nucleotide sequence represented by SEQ ID NO: 76199.

The probes having the nucleotide sequence represented by SEQ ID NOS: 92349, 92350, 92351, 92352, 92353, 92354, 92355, 92356, 92357, 92358, 92359 were selected from the ORFs having the nucleotide sequence represented by SEQ ID NO: 76200.

The probes having the nucleotide sequence represented by SEQ ID NOS: 92360, 92361, 92362, 92363, 92364, 92365, 92366, 92367, 92368, 92369, 92370 were selected from the ORFs having the nucleotide sequence represented by SEQ ID NO: 76201.

The probes having the nucleotide sequence represented by SEQ ID NOS: 92371, 92372, 92373, 92374, 92375, 92376, 92377, 92378, 92379, 92380, 92381 were selected from the ORFs having the nucleotide sequence represented by SEQ ID NO: 76202.

The probes having the nucleotide sequence represented by SEQ ID NOS: 92382, 92383, 92384, 92385, 92386, 92387, 92388, 92389, 92390, 92391, 92392 were selected from the ORFs having the nucleotide sequence represented by SEQ ID NO: 76203.

The probes having the nucleotide sequence represented by SEQ ID NOS: 92393, 92394, 92395, 92396, 92397, 92398, 92399, 92400, 92401, 92402, 92403 were selected from the ORFs having the nucleotide sequence represented by SEQ ID NO: 76204.

The probes having the nucleotide sequence represented by SEQ ID NOS: 92404, 92405, 92406, 92407, 92408, 92409, 92410, 92411, 92412, 92413, 92414 were selected from the ORFs having the nucleotide sequence represented by SEQ ID NO: 76205.

The probes having the nucleotide sequence represented by SEQ ID NOS: 92415, 92416, 92417, 92418, 92419, 92420, 92421, 92422, 92423, 92424, 92425 were selected from the ORFs having the nucleotide sequence represented by SEQ ID NO: 76206.

The probes having the nucleotide sequence represented by SEQ ID NOS: 92426, 92427, 92428, 92429, 92430, 92431, 92432, 92433, 92434, 92435, 92436 were selected from the ORFs having the nucleotide sequence represented by SEQ ID NO: 76207.

The probes having the nucleotide sequence represented by SEQ ID NOS: 92437, 92438, 92439, 92440, 92441, 92442, 92443, 92444, 92445, 92446, 92447 were selected from the ORFs having the nucleotide sequence represented by SEQ ID NO: 76208.

The probes having the nucleotide sequence represented by SEQ ID NOS: 92448, 92449, 92450, 92451, 92452, 92453, 92454, 92455, 92456, 92457, 92458 were selected from the ORFs having the nucleotide sequence represented by SEQ ID NO: 76209.

The probes having the nucleotide sequence represented by SEQ ID NOS: 92459, 92460, 92461, 92462, 92463, 92464, 92465, 92466, 92467, 92468, 92469 were selected from the ORFs having the nucleotide sequence represented by SEQ ID NO: 76210.

The probes having the nucleotide sequence represented by SEQ ID NOS: 92470, 92471, 92472, 92473, 92474, 92475, 92476, 92477, 92478, 92479, 92480 were selected from the ORFs having the nucleotide sequence represented by SEQ ID NO: 76211.

The probes having the nucleotide sequence represented by SEQ ID NOS: 92481, 92482, 92483, 92484, 92485, 92486, 92487, 92488, 92489, 92490, 92491 were selected from the ORFs having the nucleotide sequence represented by SEQ ID NO: 76212.

The probes having the nucleotide sequence represented by SEQ ID NOS: 92492, 92493, 92494, 92495, 92496, 92497, 92498, 92499, 92500, 92501, 92502 were selected from the ORFs having the nucleotide sequence represented by SEQ ID NO: 76213.

The probes having the nucleotide sequence represented by SEQ ID NOS: 92503, 92504, 92505, 92506, 92507, 92508, 92509, 92510, 92511, 92512, 92513 were selected from the ORFs having the nucleotide sequence represented by SEQ ID NO: 76214.

The probes having the nucleotide sequence represented by SEQ ID NOS: 92514, 92515, 92516, 92517, 92518, 92519, 92520, 92521, 92522, 92523, 92524 were selected from the ORFs having the nucleotide sequence represented by SEQ ID NO: 76215.

The probes having the nucleotide sequence represented by SEQ ID NOS: 92525, 92526, 92527, 92528, 92529, 92530, 92531, 92532, 92533, 92534, 92535 were selected from the ORFs having the nucleotide sequence represented by SEQ ID NO: 76216.

The probes having the nucleotide sequence represented by SEQ ID NOS: 92536, 92537, 92538, 92539, 92540, 92541, 92542, 92543, 92544, 92545, 92546 were selected from the ORFs having the nucleotide sequence represented by SEQ ID NO: 76217.

The probes having the nucleotide sequence represented by SEQ ID NOS: 92547, 92548, 92549, 92550, 92551, 92552, 92553, 92554, 92555, 92556, 92557 were selected from the ORFs having the nucleotide sequence represented by SEQ ID NO: 76218.

The probes having the nucleotide sequence represented by SEQ ID NOS: 92558, 92559, 92560, 92561, 92562, 92563, 92564, 92565, 92566, 92567, 92568 were selected from the ORFs having the nucleotide sequence represented by SEQ ID NO: 76219.

The probes having the nucleotide sequence represented by SEQ ID NOS: 92569, 92570, 92571, 92572, 92573, 92574, 92575, 92576, 92577, 92578, 92579 were selected from the ORFs having the nucleotide sequence represented by SEQ ID NO: 76220.

The probes having the nucleotide sequence represented by SEQ ID NOS: 92580, 92581, 92582, 92583, 92584, 92585, 92586, 92587, 92588, 92589, 92590 were selected from the ORFs having the nucleotide sequence represented by SEQ ID NO: 76221.

The probes having the nucleotide sequence represented by SEQ ID NOS: 92591, 92592, 92593, 92594, 92595, 92596, 92597, 92598, 92599, 92600, 92601 were selected from the ORFs having the nucleotide sequence represented by SEQ ID NO: 76222.

The probes having the nucleotide sequence represented by SEQ ID NOS: 92602, 92603, 92604, 92605, 92606, 92607, 92608, 92609, 92610, 92611, 92612 were selected from the ORFs having the nucleotide sequence represented by SEQ ID NO: 76223.

The probes having the nucleotide sequence represented by SEQ ID NOS: 92613, 92614, 92615, 92616, 92617, 92618, 92619, 92620, 92621, 92622, 92623 were selected from the ORFs having the nucleotide sequence represented by SEQ ID NO: 76224.

The probes having the nucleotide sequence represented by SEQ ID NOS: 92624, 92625, 92626, 92627, 92628, 92629, 92630, 92631, 92632, 92633, 92634 were selected from the ORFs having the nucleotide sequence represented by SEQ ID NO: 76225.

The probes having the nucleotide sequence represented by SEQ ID NOS: 92635, 92636, 92637, 92638, 92639, 92640, 92641, 92642, 92643, 92644, 92645 were selected from the ORFs having the nucleotide sequence represented by SEQ ID NO: 76226.

The probes having the nucleotide sequence represented by SEQ ID NOS: 92646, 92647, 92648, 92649, 92650, 92651, 92652, 92653, 92654, 92655, 92656 were selected from the ORFs having the nucleotide sequence represented by SEQ ID NO: 76227.

The probes having the nucleotide sequence represented by SEQ ID NOS: 92657, 92658, 92659, 92660, 92661, 92662, 92663, 92664, 92665, 92666, 92667 were selected from the ORFs having the nucleotide sequence represented by SEQ ID NO: 76228.

The probes having the nucleotide sequence represented by SEQ ID NOS: 92668, 92669, 92670, 92671, 92672, 92673, 92674, 92675, 92676, 92677, 92678 were selected from the ORFs having the nucleotide sequence represented by SEQ ID NO: 76229.

The probes having the nucleotide sequence represented by SEQ ID NOS: 92679, 92680, 92681, 92682, 92683, 92684, 92685, 92686, 92687, 92688, 92689 were selected from the ORFs having the nucleotide sequence represented by SEQ ID NO: 76230.

The probes having the nucleotide sequence represented by SEQ ID NOS: 92690, 92691, 92692, 92693, 92694, 92695, 92696, 92697, 92698, 92699, 92700 were selected from the ORFs having the nucleotide sequence represented by SEQ ID NO: 76231.

The probes having the nucleotide sequence represented by SEQ ID NOS: 92701, 92702, 92703, 92704, 92705, 92706, 92707, 92708, 92709, 92710, 92711 were selected from the ORFs having the nucleotide sequence represented by SEQ ID NO: 76232.

The probes having the nucleotide sequence represented by SEQ ID NOS: 92712, 92713, 92714, 92715, 92716, 92717, 92718, 92719, 92720, 92721, 92722, 92723, 92724, 92725, 92726, 92727, 92728, 92729, 92730, 92731, 92732, 92733 were selected from the ORFs having the nucleotide sequence represented by SEQ ID NO: 76233.

The probes having the nucleotide sequence represented by SEQ ID NOS: 92734, 92735, 92736, 92737, 92738, 92739, 92740, 92741, 92742, 92743, 92744 were selected from the ORFs having the nucleotide sequence represented by SEQ ID NO: 76234.

The probes having the nucleotide sequence represented by SEQ ID NOS: 92745, 92746, 92747, 92748, 92749, 92750, 92751, 92752, 92753, 92754, 92755 were selected-from the ORFs having the nucleotide sequence represented by SEQ ID NO: 76235.

The probes having the nucleotide sequence represented by SEQ ID NOS: 92756, 92757, 92758, 92759, 92760, 92761, 92762, 92763, 92764, 92765, 92766 were selected from the ORFs having the nucleotide sequence represented by SEQ ID NO: 76236.

The probes having the nucleotide sequence represented by SEQ ID NOS: 92767, 92768, 92769, 92770, 92771, 92772, 92773, 92774, 92775, 92776, 92777 were selected from the ORFs having the nucleotide sequence represented by SEQ ID NO: 76237.

The probes having the nucleotide sequence represented by SEQ ID NOS: 92778, 92779, 92780, 92781, 92782, 92783, 92784, 92785, 92786, 92787, 92788 were selected from the ORFs having the nucleotide sequence represented by SEQ ID NO: 76238.

The probes having the nucleotide sequence represented by SEQ ID NOS: 92789, 92790, 92791, 92792, 92793, 92794, 92795, 92796, 92797, 92798, 92799 were selected from the ORFs having the nucleotide sequence represented by SEQ ID NO: 76239.

The probes having the nucleotide sequence represented by SEQ ID NOS: 92800, 92801, 92802, 92803, 92804, 92805, 92806, 92807, 92808, 92809, 92810 were selected from the ORFs having the nucleotide sequence represented by SEQ ID NO: 76240.

The probes having the nucleotide sequence represented by SEQ ID NOS: 92811, 92812, 92813, 92814, 92815, 92816, 92817, 92818, 92819, 92820, 92821, 92822, 92823, 92824, 92825, 92826, 92827, 92828, 92829, 92830, 92831, 92832 were selected from the ORFs having the nucleotide sequence represented by SEQ ID NO: 76241.

The probes having the nucleotide sequence represented by SEQ ID NOS: 92833, 92834, 92835, 92836, 92837, 92838, 92839, 92840, 92841, 92842, 92843, 92844, 92845, 92846, 92847, 92848, 92849, 92850, 92851, 92852, 92853, 92854 were selected from the ORFs having the nucleotide sequence represented by SEQ ID NO: 76242.

The probes having the nucleotide sequence represented by SEQ ID NOS: 92855, 92856, 92857, 92858, 92859, 92860, 92861, 92862, 92863, 92864, 92865 were selected from the ORFs having the nucleotide sequence represented by SEQ ID NO: 76243.

The probes having the nucleotide sequence represented by SEQ ID NOS: 92866, 92867, 92868, 92869, 92870, 92871, 92872, 92873, 92874, 92875, 92876 were selected from the ORFs having the nucleotide sequence represented by SEQ ID NO: 76244.

The probes having the nucleotide sequence represented by SEQ ID NOS: 92877, 92878, 92879, 92880, 92881, 92882, 92883, 92884, 92885, 92886, 92887 were selected from the ORFs having the nucleotide sequence represented by SEQ ID NO: 76245.

The probes having the nucleotide sequence represented by SEQ ID NOS: 92888, 92889, 92890, 92891, 92892, 92893, 92894, 92895, 92896, 92897, 92898 were selected from the ORFs having the nucleotide sequence represented by SEQ ID NO: 76246.

The probes having the nucleotide sequence represented by SEQ ID NOS: 92899, 92900, 92901, 92902, 92903, 92904, 92905, 92906, 92907, 92908, 92909 were selected from the ORFs having the nucleotide sequence represented by SEQ ID NO: 76247.

The probes having the nucleotide sequence represented by SEQ ID NOS: 92910, 92911, 92912, 92913, 92914, 92915, 92916, 92917, 92918, 92919, 92920 were selected from the ORFs having the nucleotide sequence represented by SEQ ID NO: 76248.

The probes having the nucleotide sequence represented by SEQ ID NOS: 92921, 92922, 92923, 92924, 92925, 92926, 92927, 92928, 92929, 92930, 92931 were selected from the ORFs having the nucleotide sequence represented by SEQ ID NO: 76249.

The probes having the nucleotide sequence represented by SEQ ID NOS: 92932, 92933, 92934, 92935, 92936, 92937, 92938, 92939, 92940, 92941, 92942 were selected from the ORFs having the nucleotide sequence represented by SEQ ID NO: 76250.

The probes having the nucleotide sequence represented by SEQ ID NOS: 92943, 92944, 92945, 92946, 92947, 92948, 92949, 92950, 92951, 92952, 92953 were selected from the ORFs having the nucleotide sequence represented by SEQ ID NO: 76251.

The probes having the nucleotide sequence represented by SEQ ID NOS: 92954, 92955, 92956, 92957, 92958, 92959, 92960, 92961, 92962, 92963, 92964 were selected from the ORFs having the nucleotide sequence represented by SEQ ID NO: 76252.

The probes having the nucleotide sequence represented by SEQ ID NOS: 92965, 92966, 92967, 92968, 92969, 92970, 92971, 92972, 92973, 92974, 92975 were selected from the ORFs having the nucleotide sequence represented by SEQ ID NO: 76253.

The probes having the nucleotide sequence represented by SEQ ID NOS: 92976, 92977, 92978, 92979, 92980, 92981, 92982, 92983, 92984, 92985, 92986 were selected from the ORFs having the nucleotide sequence represented by SEQ ID NO: 76254.

The probes having the nucleotide sequence represented by SEQ ID NOS: 92987, 92988, 92989, 92990, 92991, 92992, 92993, 92994, 92995, 92996, 92997 were selected from the ORFs having the nucleotide sequence represented by SEQ ID NO: 76255.

The probes having the nucleotide sequence represented by SEQ ID NOS: 92998, 92999, 93000, 93001, 93002, 93003, 93004, 93005, 93006, 93007, 93008 were selected from the ORFs having the nucleotide sequence represented by SEQ ID NO: 76256.

The probes having the nucleotide sequence represented by SEQ ID NOS: 93009, 93010, 93011, 93012, 93013, 93014, 93015, 93016, 93017, 93018, 93019 were selected from the ORFs having the nucleotide sequence represented by SEQ ID NO: 76257.

The probes having the nucleotide sequence represented by SEQ ID NOS: 93020, 93021, 93022, 93023, 93024, 93025, 93026, 93027, 93028, 93029, 93030 were selected from the ORFs having the nucleotide sequence represented by SEQ ID NO: 76258.

The probes having the nucleotide sequence represented by SEQ ID NOS: 93031, 93032, 93033, 93034, 93035, 93036, 93037, 93038, 93039, 93040, 93041 were selected from the ORFs having the nucleotide sequence represented by SEQ ID NO: 76259.

The probes having the nucleotide sequence represented by SEQ ID NOS: 93042, 93043, 93044, 93045, 93046, 93047, 93048, 93049, 93050, 93051, 93052 were selected from the ORFs having the nucleotide sequence represented by SEQ ID NO: 76260.

The probes having the nucleotide sequence represented by SEQ ID NOS: 93053, 93054, 93055, 93056, 93057, 93058, 93059, 93060, 93061, 93062, 93063 were selected from the ORFs having the nucleotide sequence represented by SEQ ID NO: 76261.

The probes having the nucleotide sequence represented by SEQ ID NOS: 93064, 93065, 93066, 93067, 93068, 93069, 93070, 93071, 93072, 93073, 93074 were selected from the ORFs having the nucleotide sequence represented by SEQ ID NO: 76262.

The probes having the nucleotide sequence represented by SEQ ID NOS: 93075, 93076, 93077, 93078, 93079, 93080, 93081, 93082, 93083, 93084, 93085 were selected from the ORFs having the nucleotide sequence represented by SEQ ID NO: 76263.

The probes having the nucleotide sequence represented by SEQ ID NOS: 93086, 93087, 93088, 93089, 93090, 93091, 93092, 93093, 93094, 93095, 93096 were selected from the ORFs having the nucleotide sequence represented by SEQ ID NO: 76264.

The probes having the nucleotide sequence represented by SEQ ID NOS: 93097, 93098, 93099, 93100, 93101, 93102, 93103, 93104, 93105, 93106, 93107 were selected from the ORFs having the nucleotide sequence represented by SEQ ID NO: 76265.

The probes having the nucleotide sequence represented by SEQ ID NOS: 93108, 93109, 93110, 93111, 93112, 93113, 93114, 93115, 93116, 93117, 93118 were selected from the ORFs having the nucleotide sequence represented by SEQ ID NO: 76266.

The probes having the nucleotide sequence represented by SEQ ID NOS: 93119, 93120, 93121, 93122, 93123, 93124, 93125, 93126, 93127, 93128, 93129 were selected from the ORFs having the nucleotide sequence represented by SEQ ID NO: 76267.

The probes having the nucleotide sequence represented by SEQ ID NOS: 93130, 93131, 93132, 93133, 93134, 93135, 93136, 93137, 93138, 93139, 93140 were selected from the ORFs having the nucleotide sequence represented by SEQ ID NO: 76268.

The probes having the nucleotide sequence represented by SEQ ID NOS: 93141, 93142, 93143, 93144, 93145, 93146, 93147, 93148, 93149, 93150, 93151 were selected from the ORFs having the nucleotide sequence represented by SEQ ID NO: 76269.

The probes having the nucleotide sequence represented by SEQ ID NOS: 93152, 93153, 93154, 93155, 93156, 93157, 93158, 93159, 93160, 93161, 93162 were selected from the ORFs having the nucleotide sequence represented by SEQ ID NO: 76270.

The probes having the nucleotide sequence represented by SEQ ID NOS: 93163, 93164, 93165, 93166, 93167, 93168, 93169, 93170, 93171, 93172, 93173, 93174, 93175, 93176, 93177, 93178, 93179, 93180, 93181, 93182, 93183, 93184 were selected from the ORFs having the nucleotide sequence represented by SEQ ID NO: 76271.

The probes having the nucleotide sequence represented by SEQ ID NOS: 93185, 93186, 93187, 93188, 93189, 93190, 93191, 93192, 93193, 93194, 93195 were selected from the ORFs having the nucleotide sequence represented by SEQ ID NO: 76272.

The probes having the nucleotide sequence represented by SEQ ID NOS: 93196, 93197, 93198, 93199, 93200, 93201, 93202, 93203, 93204, 93205, 93206 were selected from the ORFs having the nucleotide sequence represented by SEQ ID NO: 76273.

The probes having the nucleotide sequence represented by SEQ ID NOS: 93207, 93208, 93209, 93210, 93211, 93212, 93213, 93214, 93215, 93216, 93217 were selected from the ORFs having the nucleotide sequence represented by SEQ ID NO: 76274.

The probes having the nucleotide sequence represented by SEQ ID NOS: 93218, 93219, 93220, 93221, 93222, 93223, 93224, 93225, 93226, 93227, 93228 were selected from the ORFs having the nucleotide sequence represented by SEQ ID NO: 76275.

The probes having the nucleotide sequence represented by SEQ ID NOS: 93229, 93230, 93231, 93232, 93233, 93234, 93235, 93236, 93237, 93238, 93239 were selected from the ORFs having the nucleotide sequence represented by SEQ ID NO: 76276.

The probes having the nucleotide sequence represented by SEQ ID NOS: 93240, 93241, 93242, 93243, 93244, 93245, 93246, 93247, 93248, 93249, 93250 were selected from the ORFs having the nucleotide sequence represented by SEQ ID NO: 76277.

The probes having the nucleotide sequence represented by SEQ ID NOS: 93251, 93252, 93253, 93254, 93255, 93256, 93257, 93258, 93259, 93260, 93261 were selected from the ORFs having the nucleotide sequence represented by SEQ ID NO: 76278.

The probes having the nucleotide sequence represented by SEQ ID NOS: 93262, 93263, 93264, 93265, 93266, 93267, 93268, 93269, 93270, 93271, 93272 were selected from the ORFs having the nucleotide sequence represented by SEQ ID NO: 76279.

The probes having the nucleotide sequence represented by SEQ ID NOS: 93273, 93274, 93275, 93276, 93277, 93278, 93279, 93280, 93281, 93282, 93283 were selected from the ORFs having the nucleotide sequence represented by SEQ ID NO: 76280.

The probes having the nucleotide sequence represented by SEQ ID NOS: 93284, 93285, 93286, 93287, 93288, 93289, 93290, 93291, 93292, 93293, 93294 were selected from the ORFs having the nucleotide sequence represented by SEQ ID NO: 76281.

The probes having the nucleotide sequence represented by SEQ ID NOS: 93295, 93296, 93297, 93298, 93299, 93300, 93301, 93302, 93303, 93304, 93305 were selected from the ORFs having the nucleotide sequence represented by SEQ ID NO: 76282.

The probes having the nucleotide sequence represented by SEQ ID NOS: 93306, 93307, 93308, 93309, 93310, 93311, 93312, 93313, 93314, 93315, 93316 were selected from the ORFs having the nucleotide sequence represented by SEQ ID NO: 76283.

The probes having the nucleotide sequence represented by SEQ ID NOS: 93317, 93318, 93319, 93320, 93321, 93322, 93323, 93324, 93325, 93326, 93327 were selected from the ORFs having the nucleotide sequence represented by SEQ ID NO: 76284.

The probes having the nucleotide sequence represented by SEQ ID NOS: 93328, 93329, 93330, 93331, 93332, 93333, 93334, 93335, 93336, 93337, 93338 were selected from the ORFs having the nucleotide sequence represented by SEQ ID NO: 76285.

The probes having the nucleotide sequence represented by SEQ ID NOS: 93339, 93340, 93341, 93342, 93343, 93344, 93345, 93346, 93347, 93348, 93349 were selected from the ORFs having the nucleotide sequence represented by SEQ ID NO: 76286.

The probes having the nucleotide sequence represented by SEQ ID NOS: 93350, 93351, 93352, 93353, 93354, 93355, 93356, 93357, 93358, 93359, 93360 were selected from the ORFs having the nucleotide sequence represented by SEQ ID NO: 76287.

The probes having the nucleotide sequence represented by SEQ ID NOS: 93361, 93362, 93363, 93364, 93365, 93366, 93367, 93368, 93369, 93370, 93371 were selected from the ORFs having the nucleotide sequence represented by SEQ ID NO: 76288.

The probes having the nucleotide sequence represented by SEQ ID NOS: 93372, 93373, 93374, 93375, 93376, 93377, 93378, 93379, 93380, 93381, 93382 were selected from the ORFs having the nucleotide sequence represented by SEQ ID NO: 76289.

The probes having the nucleotide sequence represented by SEQ ID NOS: 93383, 93384, 93385, 93386, 93387, 93388, 93389, 93390, 93391, 93392, 93393 were selected from the ORFs having the nucleotide sequence represented by SEQ ID NO: 76290.

The probes having the nucleotide sequence represented by SEQ ID NOS: 93394, 93395, 93396, 93397, 93398, 93399, 93400, 93401, 93402, 93403, 93404 were selected from the ORFs having the nucleotide sequence represented by SEQ ID NO: 76291.

The probes having the nucleotide sequence represented by SEQ ID NOS: 93405, 93406, 93407, 93408, 93409, 93410, 93411, 93412, 93413, 93414, 93415 were selected from the ORFs having the nucleotide sequence represented by SEQ ID NO: 76292.

The probes having the nucleotide sequence represented by SEQ ID NOS: 93416, 93417, 93418, 93419, 93420, 93421, 93422, 93423, 93424, 93425, 93426 were selected from the ORFs having the nucleotide sequence represented by SEQ ID NO: 76293.

The probes having the nucleotide sequence represented by SEQ ID NOS: 93427, 93428, 93429, 93430, 93431, 93432, 93433, 93434, 93435, 93436, 93437 were selected from the ORFs having the nucleotide sequence represented by SEQ ID NO: 76294.

The probes having the nucleotide sequence represented by SEQ ID NOS: 93438, 93439, 93440, 93441, 93442, 93443, 93444, 93445, 93446, 93447, 93448 were selected from the ORFs having the nucleotide sequence represented by SEQ ID NO: 76295.

The probes having the nucleotide sequence represented by SEQ ID NOS: 93449, 93450, 93451, 93452, 93453, 93454, 93455, 93456, 93457, 93458, 93459 were selected from the ORFs having the nucleotide sequence represented by SEQ ID NO: 76296.

The probes having the nucleotide sequence represented by SEQ ID NOS: 93460, 93461, 93462, 93463, 93464, 93465, 93466, 93467, 93468, 93469, 93470 were selected from the ORFs having the nucleotide sequence represented by SEQ ID NO: 76297.

The probes having the nucleotide sequence represented by SEQ ID NOS: 93471, 93472, 93473, 93474, 93475, 93476, 93477, 93478, 93479, 93480, 93481 were selected from the ORFs having the nucleotide sequence represented by SEQ ID NO: 76298.

The probes having the nucleotide sequence represented by SEQ ID NOS: 93482, 93483, 93484, 93485, 93486, 93487, 93488, 93489, 93490, 93491, 93492 were selected from the ORFs having the nucleotide sequence represented by SEQ ID NO: 76299.

The probes having the nucleotide sequence represented by SEQ ID NOS: 93493, 93494, 93495, 93496, 93497, 93498, 93499, 93500, 93501, 93502, 93503 were selected from the ORFs having the nucleotide sequence represented by SEQ ID NO: 76300.

The probes having the nucleotide sequence represented by SEQ ID NOS: 93504, 93505, 93506, 93507, 93508, 93509, 93510, 93511, 93512, 93513, 93514 were selected from the ORFs having the nucleotide sequence represented by SEQ ID NO: 76301.

The probes having the nucleotide sequence represented by SEQ ID NOS: 93515, 93516, 93517, 93518, 93519, 93520, 93521, 93522, 93523, 93524, 93525 were selected from the ORFs having the nucleotide sequence represented by SEQ ID NO: 76302.

The probes having the nucleotide sequence represented by SEQ ID NOS: 93526, 93527, 93528, 93529, 93530, 93531, 93532, 93533, 93534, 93535, 93536 were selected from the ORFs having the nucleotide sequence represented by SEQ ID NO: 76303.

The probes having the nucleotide sequence represented by SEQ ID NOS: 93537, 93538, 93539, 93540, 93541, 93542, 93543, 93544, 93545, 93546, 93547 were selected from the ORFs having the nucleotide sequence represented by SEQ ID NO: 76304.

The probes having the nucleotide sequence represented by SEQ ID NOS: 93548, 93549, 93550, 93551, 93552, 93553, 93554, 93555, 93556, 93557, 93558 were selected from the ORFs having the nucleotide sequence represented by SEQ ID NO: 76305.

The probes having the nucleotide sequence represented by SEQ ID NOS: 93559, 93560, 93561, 93562, 93563, 93564, 93565, 93566, 93567, 93568, 93569 were selected from the ORFs having the nucleotide sequence represented by SEQ ID NO: 76306.

The probes having the nucleotide sequence represented by SEQ ID NOS: 93570, 93571, 93572, 93573, 93574, 93575, 93576, 93577, 93578, 93579, 93580 were selected from the ORFs having the nucleotide sequence represented by SEQ ID NO: 76307.

The probes having the nucleotide sequence represented by SEQ ID NOS: 93581, 93582, 93583, 93584, 93585, 93586, 93587, 93588, 93589, 93590, 93591 were selected from the ORFs having the nucleotide sequence represented by SEQ ID NO: 76308.

The probes having the nucleotide sequence represented by SEQ ID NOS: 93592, 93593, 93594, 93595, 93596, 93597, 93598, 93599, 93600, 93601, 93602 were selected from the ORFs having the nucleotide sequence represented by SEQ ID NO: 76309.

The probes having the nucleotide sequence represented by SEQ ID NOS: 93603, 93604, 93605, 93606, 93607, 93608, 93609, 93610, 93611, 93612, 93613 were selected from the ORFs having the nucleotide sequence represented by SEQ ID NO: 76310.

The probes having the nucleotide sequence represented by SEQ ID NOS: 93614, 93615, 93616, 93617, 93618, 93619, 93620, 93621, 93622, 93623, 93624 were selected from the ORFs having the nucleotide sequence represented by SEQ ID NO: 76311.

The probes having the nucleotide sequence represented by SEQ ID NOS: 93625, 93626, 93627, 93628, 93629, 93630, 93631, 93632, 93633, 93634, 93635 were selected from the ORFs having the nucleotide sequence represented by SEQ ID NO: 76312.

The probes having the nucleotide sequence represented by SEQ ID NOS: 93636, 93637, 93638, 93639, 93640, 93641, 93642, 93643, 93644, 93645, 93646 were selected from the ORFs having the nucleotide sequence represented by SEQ ID NO: 76313.

The probes having the nucleotide sequence represented by SEQ ID NOS: 93647, 93648, 93649, 93650, 93651, 93652, 93653, 93654, 93655, 93656, 93657 were selected from the ORFs having the nucleotide sequence represented by SEQ ID NO: 76314.

The probes having the nucleotide sequence represented by SEQ ID NOS: 93658, 93659, 93660, 93661, 93662, 93663, 93664, 93665, 93666, 93667, 93668 were selected from the ORFs having the nucleotide sequence represented by SEQ ID NO: 76315.

The probes having the nucleotide sequence represented by SEQ ID NOS: 93669, 93670, 93671, 93672, 93673, 93674, 93675, 93676, 93677, 93678, 93679 were selected from the ORFs having the nucleotide sequence represented by SEQ ID NO: 76316.

The probes having the nucleotide sequence represented by SEQ ID NOS: 93680, 93681, 93682, 93683, 93684, 93685, 93686, 93687, 93688, 93689, 93690 were selected from the ORFs having the nucleotide sequence represented by SEQ ID NO: 76317.

The probes having the nucleotide sequence represented by SEQ ID NOS: 93691, 93692, 93693, 93694, 93695, 93696, 93697, 93698, 93699, 93700, 93701 were selected from the ORFs having the nucleotide sequence represented by SEQ ID NO: 76318.

The probes having the nucleotide sequence represented by SEQ ID NOS: 93702, 93703, 93704, 93705, 93706, 93707, 93708, 93709, 93710, 93711, 93712 were selected from the ORFs having the nucleotide sequence represented by SEQ ID NO: 76319.

The probes having the nucleotide sequence represented by SEQ ID NOS: 93713, 93714, 93715, 93716, 93717, 93718, 93719, 93720, 93721, 93722, 93723 were selected from the ORFs having the nucleotide sequence represented by SEQ ID NO: 76320.

The probes having the nucleotide sequence represented by SEQ ID NOS: 93724, 93725, 93726, 93727, 93728, 93729, 93730, 93731, 93732, 93733, 93734 were selected from the ORFs having the nucleotide sequence represented by SEQ ID NO: 76321.

The probes having the nucleotide sequence represented by SEQ ID NOS: 93735, 93736, 93737, 93738, 93739, 93740, 93741, 93742, 93743, 93744, 93745 were selected from the ORFs having the nucleotide sequence represented by SEQ ID NO: 76322.

The probes having the nucleotide sequence represented by SEQ ID NOS: 93746, 93747, 93748, 93749, 93750, 93751, 93752, 93753, 93754, 93755, 93756 were selected from the ORFs having the nucleotide sequence represented by SEQ ID NO: 76323.

The probes having the nucleotide sequence represented by SEQ ID NOS: 93757, 93758, 93759, 93760, 93761, 93762, 93763, 93764, 93765, 93766, 93767 were selected from the ORFs having the nucleotide sequence represented by SEQ ID NO: 76324.

The probes having the nucleotide sequence represented by SEQ ID NOS: 93768, 93769, 93770, 93771, 93772, 93773, 93774, 93775, 93776, 93777, 93778 were selected from the ORFs having the nucleotide sequence represented by SEQ ID NO: 76325.

The probes having the nucleotide sequence represented by SEQ ID NOS: 93779, 93780, 93781, 93782, 93783, 93784, 93785, 93786, 93787, 93788, 93789 were selected from the ORFs having the nucleotide sequence represented by SEQ ID NO: 76326.

The probes having the nucleotide sequence represented by SEQ ID NOS: 93790, 93791, 93792, 93793, 93794, 93795, 93796, 93797, 93798, 93799, 93800 were selected from the ORFs having the nucleotide sequence represented by SEQ ID NO: 76327.

The probes having the nucleotide sequence represented by SEQ ID NOS: 93801, 93802, 93803, 93804, 93805, 93806, 93807, 93808, 93809, 93810, 93811 were selected from the ORFs having the nucleotide sequence represented by SEQ ID NO: 76328.

The probes having the nucleotide sequence represented by SEQ ID NOS: 93812, 93813, 93814, 93815, 93816, 93817, 93818, 93819, 93820, 93821, 93822 were selected from the ORFs having the nucleotide sequence represented by SEQ ID NO: 76329.

The probes having the nucleotide sequence represented by SEQ ID NOS: 93823, 93824, 93825, 93826, 93827, 93828, 93829, 93830, 93831, 93832, 93833 were selected from the ORFs having the nucleotide sequence represented by SEQ ID NO: 76330.

The probes having the nucleotide sequence represented by SEQ ID NOS: 93834, 93835, 93836, 93837, 93838, 93839, 93840, 93841, 93842, 93843, 93844 were selected from the ORFs having the nucleotide sequence represented by SEQ ID NO: 76331.

The probes having the nucleotide sequence represented by SEQ ID NOS: 93845, 93846, 93847, 93848, 93849, 93850, 93851, 93852, 93853, 93854, 93855 were selected from the ORFs having the nucleotide sequence represented by SEQ ID NO: 76332.

The probes having the nucleotide sequence represented by SEQ ID NOS: 93856, 93857, 93858, 93859, 93860, 93861, 93862, 93863, 93864, 93865, 93866 were selected from the ORFs having the nucleotide sequence represented by SEQ ID NO: 76333.

The probes having the nucleotide sequence represented by SEQ ID NOS: 93867, 93868, 93869, 93870, 93871, 93872, 93873, 93874, 93875, 93876, 93877 were selected from the ORFs having the nucleotide sequence represented by SEQ ID NO: 76334.

The probes having the nucleotide sequence represented by SEQ ID NOS: 93878, 93879, 93880, 93881, 93882, 93883, 93884, 93885, 93886, 93887, 93888 were selected from the ORFs having the nucleotide sequence represented by SEQ ID NO: 76335.

The probes having the nucleotide sequence represented by SEQ ID NOS: 93889, 93890, 93891, 93892, 93893, 93894, 93895, 93896, 93897, 93898, 93899 were selected from the ORFs having the nucleotide sequence represented by SEQ ID NO: 76336.

The probes having the nucleotide sequence represented by SEQ ID NOS: 93900, 93901, 93902, 93903, 93904, 93905, 93906, 93907, 93908, 93909, 93910 were selected from the ORFs having the nucleotide sequence represented by SEQ ID NO: 76337.

The probes having the nucleotide sequence represented by SEQ ID NOS: 93911, 93912, 93913, 93914, 93915, 93916, 93917, 93918, 93919, 93920, 93921 were selected from the ORFs having the nucleotide sequence represented by SEQ ID NO: 76338.

The probes having the nucleotide sequence represented by SEQ ID NOS: 93922, 93923, 93924, 93925, 93926, 93927, 93928, 93929, 93930, 93931, 93932 were selected from the ORFs having the nucleotide sequence represented by SEQ ID NO: 76339.

The probes having the nucleotide sequence represented by SEQ ID NOS: 93933, 93934, 93935, 93936, 93937, 93938, 93939, 93940, 93941, 93942, 93943 were selected from the ORFs having the nucleotide sequence represented by SEQ ID NO: 76340.

The probes having the nucleotide sequence represented by SEQ ID NOS: 93944, 93945, 93946, 93947, 93948, 93949, 93950, 93951, 93952, 93953, 93954 were selected from the ORFs having the nucleotide sequence represented by SEQ ID NO: 76341.

The probes having the nucleotide sequence represented by SEQ ID NOS: 93955, 93956, 93957, 93958, 93959, 93960, 93961, 93962, 93963, 93964, 93965 were selected from the ORFs having the nucleotide sequence represented by SEQ ID NO: 76342.

The probes having the nucleotide sequence represented by SEQ ID NOS: 93966, 93967, 93968, 93969, 93970, 93971, 93972, 93973, 93974, 93975, 93976 were selected from the ORFs having the nucleotide sequence represented by SEQ ID NO: 76343.

The probes having the nucleotide sequence represented by SEQ ID NOS: 93977, 93978, 93979, 93980, 93981, 93982, 93983, 93984, 93985, 93986, 93987 were selected from the ORFs having the nucleotide sequence represented by SEQ ID NO: 76344.

The probes having the nucleotide sequence represented by SEQ ID NOS: 93988, 93989, 93990, 93991, 93992, 93993, 93994, 93995, 93996, 93997, 93998 were selected from the ORFs having the nucleotide sequence represented by SEQ ID NO: 76345.

The probes having the nucleotide sequence represented by SEQ ID NOS: 93999, 94000, 94001, 94002, 94003, 94004, 94005, 94006, 94007, 94008, 94009 were selected from the ORFs having the nucleotide sequence represented by SEQ ID NO: 76346.

The probes having the nucleotide sequence represented by SEQ ID NOS: 94010, 94011, 94012, 94013, 94014, 94015, 94016, 94017, 94018, 94019, 94020 were selected from the ORFs having the nucleotide sequence represented by SEQ ID NO: 76347.

The probes having the nucleotide sequence represented by SEQ ID NOS: 94021, 94022, 94023, 94024, 94025, 94026, 94027, 94028, 94029, 94030, 94031 were selected from the ORFs having the nucleotide sequence represented by SEQ ID NO: 76348.

The probes having the nucleotide sequence represented by SEQ ID NOS: 94032, 94033, 94034, 94035, 94036, 94037, 94038, 94039, 94040, 94041, 94042 were selected from the ORFs having the nucleotide sequence represented by SEQ ID NO: 76349.

The probes having the nucleotide sequence represented by SEQ ID NOS: 94043, 94044, 94045, 94046, 94047, 94048, 94049, 94050, 94051, 94052, 94053 were selected from the ORFs having the nucleotide sequence represented by SEQ ID NO: 76350.

The probes having the nucleotide sequence represented by SEQ ID NOS: 94054, 94055, 94056, 94057, 94058, 94059, 94060, 94061, 94062, 94063, 94064 were selected from the ORFs having the nucleotide sequence represented by SEQ ID NO: 76351.

The probes having the nucleotide sequence represented by SEQ ID NOS: 94065, 94066, 94067, 94068, 94069, 94070, 94071, 94072, 94073, 94074, 94075 were selected from the ORFs having the nucleotide sequence represented by SEQ ID NO: 76352.

The probes having the nucleotide sequence represented by SEQ ID NOS: 94076, 94077, 94078, 94079, 94080, 94081, 94082, 94083, 94084, 94085, 94086 were selected from the ORFs having the nucleotide sequence represented by SEQ ID NO: 76353.

The probes having the nucleotide sequence represented by SEQ ID NOS: 94087, 94088, 94089, 94090, 94091, 94092, 94093, 94094, 94095, 94096, 94097 were selected from the ORFs having the nucleotide sequence represented by SEQ ID NO: 76354.

The probes having the nucleotide sequence represented by SEQ ID NOS: 94098, 94099, 94100, 94101, 94102, 94103, 94104, 94105, 94106, 94107, 94108 were selected from the ORFs having the nucleotide sequence represented by SEQ ID NO: 76355.

The probes having the nucleotide sequence represented by SEQ ID NOS: 94109, 94110, 94111, 94112, 94113, 94114, 94115, 94116, 94117, 94118, 94119 were selected from the ORFs having the nucleotide sequence represented by SEQ ID NO: 76356.

The probes having the nucleotide sequence represented by SEQ ID NOS: 94120, 94121, 94122, 94123, 94124, 94125, 94126, 94127, 94128, 94129, 94130 were selected from the ORFs having the nucleotide sequence represented by SEQ ID NO: 76357.

The probes having the nucleotide sequence represented by SEQ ID NOS: 94131, 94132, 94133, 94134, 94135, 94136, 94137, 94138, 94139, 94140, 94141 were selected from the ORFs having the nucleotide sequence represented by SEQ ID NO: 76358.

The probes having the nucleotide sequence represented by SEQ ID NOS: 94142, 94143, 94144, 94145, 94146, 94147, 94148, 94149, 94150, 94151, 94152 were selected from the ORFs having the nucleotide sequence represented by SEQ ID NO: 76359.

The probes having the nucleotide sequence represented by SEQ ID NOS: 94153, 94154, 94155, 94156, 94157, 94158, 94159, 94160, 94161, 94162, 94163 were selected from the ORFs having the nucleotide sequence represented by SEQ ID NO: 76360.

The probes having the nucleotide sequence represented by SEQ ID NOS: 94164, 94165, 94166, 94167, 94168, 94169, 94170, 94171, 94172, 94173, 94174 were selected from the ORFs having the nucleotide sequence represented by SEQ ID NO: 76361.

The probes having the nucleotide sequence represented by SEQ ID NOS: 94175, 94176, 94177, 94178, 94179, 94180, 94181, 94182, 94183, 94184, 94185 were selected from the ORFs having the nucleotide sequence represented by SEQ ID NO: 76362.

The probes having the nucleotide sequence represented by SEQ ID NOS: 94186, 94187, 94188, 94189, 94190, 94191, 94192, 94193, 94194, 94195, 94196 were selected from the ORFs having the nucleotide sequence represented by SEQ ID NO: 76363.

The probes having the nucleotide sequence represented by SEQ ID NOS: 94197, 94198, 94199, 94200, 94201, 94202, 94203, 94204, 94205, 94206, 94207 were selected from the ORFs having the nucleotide sequence represented by SEQ ID NO: 76364.

The probes having the nucleotide sequence represented by SEQ ID NOS: 94208, 94209, 94210, 94211, 94212, 94213, 94214, 94215, 94216, 94217, 94218 were selected from the ORFs having the nucleotide sequence represented by SEQ ID NO: 76365.

The probes having the nucleotide sequence represented by SEQ ID NOS: 94219, 94220, 94221, 94222, 94223, 94224, 94225, 94226, 94227, 94228, 94229 were selected from the ORFs having the nucleotide sequence represented by SEQ ID NO: 76366.

The probes having the nucleotide sequence represented by SEQ ID NOS: 94230, 94231, 94232, 94233, 94234, 94235, 94236, 94237, 94238, 94239, 94240 were selected from the ORFs having the nucleotide sequence represented by SEQ ID NO: 76367.

The probes having the nucleotide sequence represented by SEQ ID NOS: 94241, 94242, 94243, 94244, 94245, 94246, 94247, 94248, 94249, 94250, 94251 were selected from the ORFs having the nucleotide sequence represented by SEQ ID NO: 76368.

The probes having the nucleotide sequence represented by SEQ ID NOS: 94252, 94253, 94254, 94255, 94256, 94257, 94258, 94259, 94260, 94261, 94262 were selected from the ORFs having the nucleotide sequence represented by SEQ ID NO: 76369.

The probes having the nucleotide sequence represented by SEQ ID NOS: 94263, 94264, 94265, 94266, 94267, 94268, 94269, 94270, 94271, 94272, 94273 were selected from the ORFs having the nucleotide sequence represented by SEQ ID NO: 76370.

The probes having the nucleotide sequence represented by SEQ ID NOS: 94274, 94275, 94276, 94277, 94278, 94279, 94280, 94281, 94282, 94283, 94284 were selected from the ORFs having the nucleotide sequence represented by SEQ ID NO: 76371.

The probes having the nucleotide sequence represented by SEQ ID NOS: 94285, 94286, 94287, 94288, 94289, 94290, 94291, 94292, 94293, 94294, 94295 were selected from the ORFs having the nucleotide sequence represented by SEQ ID NO: 76372.

The probes having the nucleotide sequence represented by SEQ ID NOS: 94296, 94297, 94298, 94299, 94300, 94301, 94302, 94303, 94304, 94305, 94306 were selected from the ORFs having the nucleotide sequence represented by SEQ ID NO: 76373.

The probes having the nucleotide sequence represented by SEQ ID NOS: 94307, 94308, 94309, 94310, 94311, 94312, 94313, 94314, 94315, 94316, 94317 were selected from the ORFs having the nucleotide sequence represented by SEQ ID NO: 76374.

The probes having the nucleotide sequence represented by SEQ ID NOS: 94318, 94319, 94320, 94321, 94322, 94323, 94324, 94325, 94326, 94327, 94328 were selected from the ORFs having the nucleotide sequence represented by SEQ ID NO: 76375.

The probes having the nucleotide sequence represented by SEQ ID NOS: 94329, 94330, 94331, 94332, 94333, 94334, 94335, 94336, 94337, 94338, 94339 were selected from the ORFs having the nucleotide sequence represented by SEQ ID NO: 76376.

The probes having the nucleotide sequence represented by SEQ ID NOS: 94340, 94341, 94342, 94343, 94344, 94345, 94346, 94347, 94348, 94349, 94350 were selected from the ORFs having the nucleotide sequence represented by SEQ ID NO: 76377.

The probes having the nucleotide sequence represented by SEQ ID NOS: 94351, 94352, 94353, 94354, 94355, 94356, 94357, 94358, 94359, 94360, 94361 were selected from the ORFs having the nucleotide sequence represented by SEQ ID NO: 76378.

The probes having the nucleotide sequence represented by SEQ ID NOS: 94362, 94363, 94364, 94365, 94366, 94367, 94368, 94369, 94370, 94371, 94372 were selected from the ORFs having the nucleotide sequence represented by SEQ ID NO: 76379.

The probes having the nucleotide sequence represented by SEQ ID NOS: 94373, 94374, 94375, 94376, 94377, 94378, 94379, 94380, 94381, 94382, 94383 were selected from the ORFs having the nucleotide sequence represented by SEQ ID NO: 76380.

The probes having the nucleotide sequence represented by SEQ ID NOS: 94384, 94385, 94386, 94387, 94388, 94389, 94390, 94391, 94392, 94393, 94394 were selected from the ORFs having the nucleotide sequence represented by SEQ ID NO: 76381.

The probes having the nucleotide sequence represented by SEQ ID NOS: 94417, 94418, 94419, 94420, 94421, 94422, 94423, 94424, 94425, 94426, 94427 were selected from the ORFs having the nucleotide sequence represented by SEQ ID NO: 76382.

The probes having the nucleotide sequence represented by SEQ ID NOS: 94395, 94396, 94397, 94398, 94399, 94400, 94401, 94402, 94403, 94404, 94405 were selected from the ORFs having the nucleotide sequence represented by SEQ ID NO: 76383.

The probes having the nucleotide sequence represented by SEQ ID NOS: 94406, 94407, 94408, 94409, 94410, 94411, 94412, 94413, 94414, 94415, 94416 were selected from the ORFs having the nucleotide sequence represented by SEQ ID NO: 76384.

The probes having the nucleotide sequence represented by SEQ ID NOS: 94428, 94429, 94430, 94431, 94432, 94433, 94434, 94435, 94436, 94437, 94438 were selected from the ORFs having the nucleotide sequence represented by SEQ ID NO: 76385.

The probes having the nucleotide sequence represented by SEQ ID NOS: 94439, 94440, 94441, 94442, 94443, 94444, 94445, 94446, 94447, 94448, 94449 were selected from the ORFs having the nucleotide sequence represented by SEQ ID NO: 76386.

The probes having the nucleotide sequence represented by SEQ ID NOS: 94450, 94451, 94452, 94453, 94454, 94455, 94456, 94457, 94458, 94459, 94460 were selected from the ORFs having the nucleotide sequence represented by SEQ ID NO: 76387.

The probes having the nucleotide sequence represented by SEQ ID NOS: 94461, 94462, 94463, 94464, 94465, 94466, 94467, 94468, 94469, 94470, 94471 were selected from the ORFs having the nucleotide sequence represented by SEQ ID NO: 76388.

The probes having the nucleotide sequence represented by SEQ ID NOS: 94472, 94473, 94474, 94475, 94476, 94477, 94478, 94479, 94480, 94481, 94482 were selected from the ORFs having the nucleotide sequence represented by SEQ ID NO: 76389.

The probes having the nucleotide sequence represented by SEQ ID NOS: 94483, 94484, 94485, 94486, 94487, 94488, 94489, 94490, 94491, 94492, 94493 were selected from the ORFs having the nucleotide sequence represented by SEQ ID NO: 76390.

The probes having the nucleotide sequence represented by SEQ ID NOS: 94494, 94495, 94496, 94497, 94498, 94499, 94500, 94501, 94502, 94503, 94504 were selected from the ORFs having the nucleotide sequence represented by SEQ ID NO: 76391.

The probes having the nucleotide sequence represented by SEQ ID NOS: 94505, 94506, 94507, 94508, 94509, 94510, 94511, 94512, 94513, 94514, 94515 were selected from the ORFs having the nucleotide sequence represented by SEQ ID NO: 76392.

The probes having the nucleotide sequence represented by SEQ ID NOS: 94516, 94517, 94518, 94519, 94520, 94521, 94522, 94523, 94524, 94525, 94526 were selected from the ORFs having the nucleotide sequence represented by SEQ ID NO: 76393.

The probes having the nucleotide sequence represented by SEQ ID NOS: 94527, 94528, 94529, 94530, 94531, 94532, 94533, 94534, 94535, 94536, 94537 were selected from the ORFs having the nucleotide sequence represented by SEQ ID NO: 76394.

The probes having the nucleotide sequence represented by SEQ ID NOS: 94538, 94539, 94540, 94541, 94542, 94543, 94544, 94545, 94546, 94547, 94548 were selected from the ORFs having the nucleotide sequence represented by SEQ ID NO: 76395.

The probes having the nucleotide sequence represented by SEQ ID NOS: 94549, 94550, 94551, 94552, 94553, 94554, 94555, 94556, 94557, 94558, 94559 were selected from the ORFs having the nucleotide sequence represented by SEQ ID NO: 76396.

The probes having the nucleotide sequence represented by SEQ ID NOS: 94560, 94561, 94562, 94563, 94564, 94565, 94566, 94567, 94568, 94569, 94570 were selected from the ORFs having the nucleotide sequence represented by SEQ ID NO: 76397.

The probes having the nucleotide sequence represented by SEQ ID NOS: 94571, 94572, 94573, 94574, 94575, 94576, 94577, 94578, 94579, 94580, 94581 were selected from the ORFs having the nucleotide sequence represented by SEQ ID NO: 76398.

The probes having the nucleotide sequence represented by SEQ ID NOS: 94582, 94583, 94584, 94585, 94586, 94587, 94588, 94589, 94590, 94591, 94592 were selected from the ORFs having the nucleotide sequence represented by SEQ ID NO: 76399.

The probes having the nucleotide sequence represented by SEQ ID NOS: 94593, 94594, 94595, 94596, 94597, 94598, 94599, 94600, 94601, 94602, 94603 were selected from the ORFs having the nucleotide sequence represented by SEQ ID NO: 76400.

The probes having the nucleotide sequence represented by SEQ ID NOS: 94604, 94605, 94606, 94607, 94608, 94609, 94610, 94611, 94612, 94613, 94614 were selected from the ORFs having the nucleotide sequence represented by SEQ ID NO: 76401.

The probes having the nucleotide sequence represented by SEQ ID NOS: 94615, 94616, 94617, 94618, 94619, 94620, 94621, 94622, 94623, 94624, 94625 were selected from the ORFs having the nucleotide sequence represented by SEQ ID NO: 76402.

The probes having the nucleotide sequence represented by SEQ ID NOS: 94626, 94627, 94628, 94629, 94630, 94631, 94632, 94633, 94634, 94635, 94636 were selected from the ORFs having the nucleotide sequence represented by SEQ ID NO: 76403.

The probes having the nucleotide sequence represented by SEQ ID NOS: 94637, 94638, 94639, 94640, 94641, 94642, 94643, 94644, 94645, 94646, 94647 were selected from the ORFs having the nucleotide sequence represented by SEQ ID NO: 76404.

The probes having the nucleotide sequence represented by SEQ ID NOS: 94648, 94649, 94650, 94651, 94652, 94653, 94654, 94655, 94656, 94657, 94658 were selected from the ORFs having the nucleotide sequence represented by SEQ ID NO: 76405.

The probes having the nucleotide sequence represented by SEQ ID NOS: 94659, 94660, 94661, 94662, 94663, 94664, 94665, 94666, 94667, 94668, 94669 were selected from the ORFs having the nucleotide sequence represented by SEQ ID NO: 76406.

The probes having the nucleotide sequence represented by SEQ ID NOS: 94670, 94671, 94672, 94673, 94674, 94675, 94676, 94677, 94678, 94679, 94680 were selected from the ORFs having the nucleotide sequence represented by SEQ ID NO: 76407.

The probes having the nucleotide sequence represented by SEQ ID NOS: 94681, 94682, 94683, 94684, 94685, 94686, 94687, 94688, 94689, 94690, 94691 were selected from the ORFs having the nucleotide sequence represented by SEQ ID NO: 76408.

The probes having the nucleotide sequence represented by SEQ ID NOS: 94692, 94693, 94694, 94695, 94696, 94697, 94698, 94699, 94700, 94701, 94702 were selected from the ORFs having the nucleotide sequence represented by SEQ ID NO: 76409.

The probes having the nucleotide sequence represented by SEQ ID NOS: 94703, 94704, 94705, 94706, 94707, 94708, 94709, 94710, 94711, 94712, 94713 were selected from the ORFs having the nucleotide sequence represented by SEQ ID NO: 76410.

The probes having the nucleotide sequence represented by SEQ ID NOS: 94714, 94715, 94716, 94717, 94718, 94719, 94720, 94721, 94722, 94723, 94724 were selected from the ORFs having the nucleotide sequence represented by SEQ ID NO: 76411.

The probes having the nucleotide sequence represented by SEQ ID NOS: 94725, 94726, 94727, 94728, 94729, 94730, 94731, 94732, 94733, 94734, 94735 were selected from the ORFs having the nucleotide sequence represented by SEQ ID NO: 76412.

The probes having the nucleotide sequence represented by SEQ ID NOS: 94736, 94737, 94738, 94739, 94740, 94741, 94742, 94743, 94744, 94745, 94746 were selected from the ORFs having the nucleotide sequence represented by SEQ ID NO: 76413.

The probes having the nucleotide sequence represented by SEQ ID NOS: 94747, 94748, 94749, 94750, 94751, 94752, 94753, 94754, 94755, 94756, 94757 were selected from the ORFs having the nucleotide sequence represented by SEQ ID NO: 76414.

The probes having the nucleotide sequence represented by SEQ ID NOS: 94758, 94759, 94760, 94761, 94762, 94763, 94764, 94765, 94766, 94767, 94768 were selected from the ORFs having the nucleotide sequence represented by SEQ ID NO: 76415.

The probes having the nucleotide sequence represented by SEQ ID NOS: 94769, 94770, 94771, 94772, 94773, 94774, 94775, 94776, 94777, 94778, 94779 were selected from the ORFs having the nucleotide sequence represented by SEQ ID NO: 76416.

The probes having the nucleotide sequence represented by SEQ ID NOS: 94780, 94781, 94782, 94783, 94784, 94785, 94786, 94787, 94788, 94789, 94790 were selected from the ORFs having the nucleotide sequence represented by SEQ ID NO: 76417.

The probes having the nucleotide sequence represented by SEQ ID NOS: 94791, 94792, 94793, 94794, 94795, 94796, 94797, 94798, 94799, 94800, 94801 were selected from the ORFs having the nucleotide sequence represented by SEQ ID NO: 76418.

The probes having the nucleotide sequence represented by SEQ ID NOS: 94802, 94803, 94804, 94805, 94806, 94807, 94808, 94809, 94810, 94811, 94812 were selected from the ORFs having the nucleotide sequence represented by SEQ ID NO: 76419.

The probes having the nucleotide sequence represented by SEQ ID NOS: 94813, 94814, 94815, 94816, 94817, 94818, 94819, 94820, 94821, 94822, 94823 were selected from the ORFs having the nucleotide sequence represented by SEQ ID NO: 76420.

The probes having the nucleotide sequence represented by SEQ ID NOS: 94824, 94825, 94826, 94827, 94828, 94829, 94830, 94831, 94832, 94833, 94834 were selected from the ORFs having the nucleotide sequence represented by SEQ ID NO: 76421.

The probes having the nucleotide sequence represented by SEQ ID NOS: 94835, 94836, 94837, 94838, 94839, 94840, 94841, 94842, 94843, 94844, 94845 were selected from the ORFs having the nucleotide sequence represented by SEQ ID NO: 76422.

The probes having the nucleotide sequence represented by SEQ ID NOS: 94846, 94847, 94848, 94849, 94850, 94851, 94852, 94853, 94854, 94855, 94856 were selected from the ORFs having the nucleotide sequence represented by SEQ ID NO: 76423.

The probes having the nucleotide sequence represented by SEQ ID NOS: 94857, 94858, 94859, 94860, 94861, 94862, 94863, 94864, 94865, 94866, 94867 were selected from the ORFs having the nucleotide sequence represented by SEQ ID NO: 76424.

The probes having the nucleotide sequence represented by SEQ ID NOS: 94868, 94869, 94870, 94871, 94872, 94873, 94874, 94875, 94876, 94877, 94878 were selected from the ORFs having the nucleotide sequence represented by SEQ ID NO: 76425.

The probes having the nucleotide sequence represented by SEQ ID NOS: 94879, 94880, 94881, 94882, 94883, 94884, 94885, 94886, 94887, 94888, 94889 were selected from the ORFs having the nucleotide sequence represented by SEQ ID NO: 76426.

The probes having the nucleotide sequence represented by SEQ ID NOS: 94890, 94891, 94892, 94893, 94894, 94895, 94896, 94897, 94898, 94899, 94900 were selected from the ORFs having the nucleotide sequence represented by SEQ ID NO: 76427.

The probes having the nucleotide sequence represented by SEQ ID NOS: 94901, 94902, 94903, 94904, 94905, 94906, 94907, 94908, 94909, 94910, 94911 were selected from the ORFs having the nucleotide sequence represented by SEQ ID NO: 76428.

The probes having the nucleotide sequence represented by SEQ ID NOS: 94912, 94913, 94914, 94915, 94916, 94917, 94918, 94919, 94920, 94921, 94922 were selected from the ORFs having the nucleotide sequence represented by SEQ ID NO: 76429.

The probes having the nucleotide sequence represented by SEQ ID NOS: 94923, 94924, 94925, 94926, 94927, 94928, 94929, 94930, 94931, 94932, 94933 were selected from the ORFs having the nucleotide sequence represented by SEQ ID NO: 76430.

The probes having the nucleotide sequence represented by SEQ ID NOS: 94934, 94935, 94936, 94937, 94938, 94939, 94940, 94941, 94942, 94943, 94944 were selected from the ORFs having the nucleotide sequence represented by SEQ ID NO: 76431.

The probes having the nucleotide sequence represented by SEQ ID NOS: 94945, 94946, 94947, 94948, 94949, 94950, 94951, 94952, 94953, 94954, 94955 were selected from the ORFs having the nucleotide sequence represented by SEQ ID NO: 76432.

The probes having the nucleotide sequence represented by SEQ ID NOS: 94956, 94957, 94958, 94959, 94960, 94961, 94962, 94963, 94964, 94965, 94966 were selected from the ORFs having the nucleotide sequence represented by SEQ ID NO: 76433.

The probes having the nucleotide sequence represented by SEQ ID NOS: 94967, 94968, 94969, 94970, 94971, 94972, 94973, 94974, 94975, 94976, 94977 were selected from the ORFs having the nucleotide sequence represented by SEQ ID NO: 76434.

The probes having the nucleotide sequence represented by SEQ ID NOS: 94978, 94979, 94980, 94981, 94982, 94983, 94984, 94985, 94986, 94987, 94988 were selected from the ORFs having the nucleotide sequence represented by SEQ ID NO: 76435.

The probes having the nucleotide sequence represented by SEQ ID NOS: 94989, 94990, 94991, 94992, 94993, 94994, 94995, 94996, 94997, 94998, 94999 were selected from the ORFs having the nucleotide sequence represented by SEQ ID NO: 76436.

The probes having the nucleotide sequence represented by SEQ ID NOS: 95000, 95001, 95002, 95003, 95004, 95005, 95006, 95007, 95008, 95009, 95010 were selected from the ORFs having the nucleotide sequence represented by SEQ ID NO: 76437.

The probes having the nucleotide sequence represented by SEQ ID NOS: 95011, 95012, 95013, 95014, 95015, 95016, 95017, 95018, 95019, 95020, 95021 were selected from the ORFs having the nucleotide sequence represented by SEQ ID NO: 76438.

The probes having the nucleotide sequence represented by SEQ ID NOS: 95022, 95023, 95024, 95025, 95026, 95027, 95028, 95029, 95030, 95031, 95032 were selected from the ORFs having the nucleotide sequence represented by SEQ ID NO: 76439.

The probes having the nucleotide sequence represented by SEQ ID NOS: 95033, 95034, 95035, 95036, 95037, 95038, 95039, 95040, 95041, 95042, 95043 were selected from the ORFs having the nucleotide sequence represented by SEQ ID NO: 76440.

The probes having the nucleotide sequence represented by SEQ ID NOS: 95044, 95045, 95046, 95047, 95048, 95049, 95050, 95051, 95052, 95053, 95054 were selected from the ORFs having the nucleotide sequence represented by SEQ ID NO: 76441.

The probes having the nucleotide sequence represented by SEQ ID NOS: 95055, 95056, 95057, 95058, 95059, 95060, 95061, 95062, 95063, 95064, 95065 were selected from the ORFs having the nucleotide sequence represented by SEQ ID NO: 76442.

The probes having the nucleotide sequence represented by SEQ ID NOS: 95066, 95067, 95068, 95069, 95070, 95071, 95072, 95073, 95074, 95075, 95076 were selected from the ORFs having the nucleotide sequence represented by SEQ ID NO: 76443.

The probes having the nucleotide sequence represented by SEQ ID NOS: 95077, 95078, 95079, 95080, 95081, 95082, 95083, 95084, 95085, 95086, 95087 were selected from the ORFs having the nucleotide sequence represented by SEQ ID NO: 76444.

The probes having the nucleotide sequence represented by SEQ ID NOS: 95088, 95089, 95090, 95091, 95092, 95093, 95094, 95095, 95096, 95097, 95098 were selected from the ORFs having the nucleotide sequence represented by SEQ ID NO: 76445.

The probes having the nucleotide sequence represented by SEQ ID NOS: 95099, 95100, 95101, 95102, 95103, 95104, 95105, 95106, 95107, 95108, 95109 were selected from the ORFs having the nucleotide sequence represented by SEQ ID NO: 76446.

The probes having the nucleotide sequence represented by SEQ ID NOS: 95110, 95111, 95112, 95113, 95114, 95115, 95116, 95117, 95118, 95119, 95120 were selected from the ORFs having the nucleotide sequence represented by SEQ ID NO: 76447.

The probes having the nucleotide sequence represented by SEQ ID NOS: 95121, 95122, 95123, 95124, 95125, 95126, 95127, 95128, 95129, 95130, 95131 were selected from the ORFs having the nucleotide sequence represented by SEQ ID NO: 76448.

The probes having the nucleotide sequence represented by SEQ ID NOS: 95132, 95133, 95134, 95135, 95136, 95137, 95138, 95139, 95140, 95141, 95142 were selected from the ORFs having the nucleotide sequence represented by SEQ ID NO: 76449.

The probes having the nucleotide sequence represented by SEQ ID NOS: 95143, 95144, 95145, 95146, 95147, 95148, 95149, 95150, 95151, 95152, 95153 were selected from the ORFs having the nucleotide sequence represented by SEQ ID NO: 76450.

The probes having the nucleotide sequence represented by SEQ ID NOS: 95154, 95155, 95156, 95157, 95158, 95159, 95160, 95161, 95162, 95163, 95164 were selected from the ORFs having the nucleotide sequence represented by SEQ ID NO: 76451.

The probes having the nucleotide sequence represented by SEQ ID NOS: 95165, 95166, 95167, 95168, 95169, 95170, 95171, 95172, 95173, 95174, 95175 were selected from the ORFs having the nucleotide sequence represented by SEQ ID NO: 76452.

The probes having the nucleotide sequence represented by SEQ ID NOS: 95176, 95177, 95178, 95179, 95180, 95181, 95182, 95183, 95184, 95185, 95186 were selected from the ORFs having the nucleotide sequence represented by SEQ ID NO: 76453.

The probes having the nucleotide sequence represented by SEQ ID NOS: 95187, 95188, 95189, 95190, 95191, 95192, 95193, 95194, 95195, 95196, 95197 were selected from the ORFs having the nucleotide sequence represented by SEQ ID NO: 76454.

The probes having the nucleotide sequence represented by SEQ ID NOS: 95198, 95199, 95200, 95201, 95202, 95203, 95204, 95205, 95206, 95207, 95208 were selected from the ORFs having the nucleotide sequence represented by SEQ ID NO: 76455.

The probes having the nucleotide sequence represented by SEQ ID-NOS: 95209, 95210, 95211, 95212, 95213, 95214, 95215, 95216, 95217, 95218, 95219 were selected from the ORFs having the nucleotide sequence represented by SEQ ID NO: 76456.

The probes having the nucleotide sequence represented by SEQ ID NOS: 95220, 95221, 95222, 95223, 95224, 95225, 95226, 95227, 95228, 95229, 95230 were selected from the ORFs having the nucleotide sequence represented by SEQ ID NO: 76457.

The probes having the nucleotide sequence represented by SEQ ID NOS: 95231, 95232, 95233, 95234, 95235, 95236, 95237, 95238, 95239, 95240, 95241 were selected from the ORFs having the nucleotide sequence represented by SEQ ID NO: 76458.

The probes having the nucleotide sequence represented by SEQ ID NOS: 95242, 95243, 95244, 95245, 95246, 95247, 95248, 95249, 95250, 95251, 95252 were selected from the ORFs having the nucleotide sequence represented by SEQ ID NO: 76459.

The probes having the nucleotide sequence represented by SEQ ID NOS: 95253, 95254, 95255, 95256, 95257, 95258, 95259, 95260, 95261, 95262, 95263 were selected from the ORFs having the nucleotide sequence represented by SEQ ID NO: 76460.

The probes having the nucleotide sequence represented by SEQ ID NOS: 95264, 95265, 95266, 95267, 95268, 95269, 95270, 95271, 95272, 95273, 95274 were selected from the ORFs having the nucleotide sequence represented by SEQ ID NO: 76461.

The probes having the nucleotide sequence represented by SEQ ID NOS: 95275, 95276, 95277, 95278, 95279, 95280, 95281, 95282, 95283, 95284, 95285 were selected from the ORFs having the nucleotide sequence represented by SEQ ID NO: 76462.

The probes having the nucleotide sequence represented by SEQ ID NOS: 95286, 95287, 95288, 95289, 95290, 95291, 95292, 95293, 95294, 95295, 95296 were selected from the ORFs having the nucleotide sequence represented by SEQ ID NO: 76463.

The probes having the nucleotide sequence represented by SEQ ID NOS: 95297, 95298, 95299, 95300, 95301, 95302, 95303, 95304, 95305, 95306, 95307 were selected from the ORFs having the nucleotide sequence represented by SEQ ID NO: 76464.

The probes having the nucleotide sequence represented by SEQ ID NOS: 95308, 95309, 95310, 95311, 95312, 95313, 95314, 95315, 95316, 95317, 95318 were selected from the ORFs having the nucleotide sequence represented by SEQ ID NO: 76465.

The probes having the nucleotide sequence represented by SEQ ID NOS: 95319, 95320, 95321, 95322, 95323, 95324, 95325, 95326, 95327, 95328, 95329 were selected from the ORFs having the nucleotide sequence represented by SEQ ID NO: 76466.

The probes having the nucleotide sequence represented by SEQ ID NOS: 95330, 95331, 95332, 95333, 95334, 95335, 95336, 95337, 95338, 95339, 95340 were selected from the ORFs having the nucleotide sequence represented by SEQ ID NO: 76467.

The probes having the nucleotide sequence represented by SEQ ID NOS: 95341, 95342, 95343, 95344, 95345, 95346, 95347, 95348, 95349, 95350, 95351 were selected from the ORFs having the nucleotide sequence represented by SEQ ID NO: 76468.

The probes having the nucleotide sequence represented by SEQ ID NOS: 95352, 95353, 95354, 95355, 95356, 95357, 95358, 95359, 95360, 95361, 95362 were selected from the ORFs having the nucleotide sequence represented by SEQ ID NO: 76469.

The probes having the nucleotide sequence represented by SEQ ID NOS: 95363, 95364, 95365, 95366, 95367, 95368, 95369, 95370, 95371, 95372, 95373 were selected from the ORFs having the nucleotide sequence represented by SEQ ID NO: 76470.

The probes having the nucleotide sequence represented by SEQ ID NOS: 95374, 95375, 95376, 95377, 95378, 95379, 95380, 95381, 95382, 95383, 95384 were selected from the ORFs having the nucleotide sequence represented by SEQ ID NO: 76471.

The probes having the nucleotide sequence represented by SEQ ID NOS: 95385, 95386, 95387, 95388, 95389, 95390, 95391, 95392, 95393, 95394, 95395 were selected from the ORFs having the nucleotide sequence represented by SEQ ID NO: 76472.

The probes having the nucleotide sequence represented by SEQ ID NOS: 95396, 95397, 95398, 95399, 95400, 95401, 95402, 95403, 95404, 95405, 95406 were selected from the ORFs having the nucleotide sequence represented by SEQ ID NO: 76473.

The probes having the nucleotide sequence represented by SEQ ID NOS: 95407, 95408, 95409, 95410, 95411, 95412, 95413, 95414, 95415, 95416, 95417 were selected from the ORFs having the nucleotide sequence represented by SEQ ID NO: 76474.

The probes having the nucleotide sequence represented by SEQ ID NOS: 95418, 95419, 95420, 95421, 95422, 95423, 95424, 95425, 95426, 95427, 95428 were selected from the ORFs having the nucleotide sequence represented by SEQ ID NO: 76475.

The probes having the nucleotide sequence represented by SEQ ID NOS: 95429, 95430, 95431, 95432, 95433, 95434, 95435, 95436, 95437, 95438, 95439 were selected from the ORFs having the nucleotide sequence represented by SEQ ID NO: 76476.

The probes having the nucleotide sequence represented by SEQ ID NOS: 95440, 95441, 95442, 95443, 95444, 95445, 95446, 95447, 95448, 95449, 95450 were selected from the ORFs having the nucleotide sequence represented by SEQ ID NO: 76477.

The probes having the nucleotide sequence represented by SEQ ID NOS: 95451, 95452, 95453, 95454, 95455, 95456, 95457, 95458, 95459, 95460, 95461 were selected from the ORFs having the nucleotide sequence represented by SEQ ID NO: 76478.

The probes having the nucleotide sequence represented by SEQ ID NOS: 95462, 95463, 95464, 95465, 95466, 95467, 95468, 95469, 95470, 95471, 95472 were selected from the ORFs having the nucleotide sequence represented by SEQ ID NO: 76479.

The probes having the nucleotide sequence represented by SEQ ID NOS: 95473, 95474, 95475, 95476, 95477, 95478, 95479, 95480, 95481, 95482, 95483 were selected from the ORFs having the nucleotide sequence represented by SEQ ID NO: 76480.

The probes having the nucleotide sequence represented by SEQ ID NOS: 95484, 95485, 95486, 95487, 95488, 95489, 95490, 95491, 95492, 95493, 95494 were selected from the ORFs having the nucleotide sequence represented by SEQ ID NO: 76481.

The probes having the nucleotide sequence represented by SEQ ID NOS: 95495, 95496, 95497, 95498, 95499, 95500, 95501, 95502, 95503, 95504, 95505 were selected from the ORFs having the nucleotide sequence represented by SEQ ID NO: 76482.

The probes having the nucleotide sequence represented by SEQ ID NOS: 95506, 95507, 95508, 95509, 95510, 95511, 95512, 95513, 95514, 95515, 95516 were selected from the ORFs having the nucleotide sequence represented by SEQ ID NO: 76483.

The probes having the nucleotide sequence represented by SEQ ID NOS: 95517, 95518, 95519, 95520, 95521, 95522, 95523, 95524, 95525, 95526, 95527 were selected from the ORFs having the nucleotide sequence represented by SEQ ID NO: 76484.

The probes having the nucleotide sequence represented by SEQ ID NOS: 95528, 95529, 95530, 95531, 95532, 95533, 95534, 95535, 95536, 95537, 95538 were selected from the ORFs having the nucleotide sequence represented by SEQ ID NO: 76485.

The probes having the nucleotide sequence represented by SEQ ID NOS: 95539, 95540, 95541, 95542, 95543, 95544, 95545, 95546, 95547, 95548, 95549 were selected from the ORFs having the nucleotide sequence represented by SEQ ID NO: 76486.

The probes having the nucleotide sequence represented by SEQ ID NOS: 95550, 95551, 95552, 95553, 95554, 95555, 95556, 95557, 95558, 95559, 95560 were selected from the ORFs having the nucleotide sequence represented by SEQ ID NO: 76487.

The probes having the nucleotide sequence represented by SEQ ID NOS: 95561, 95562, 95563, 95564, 95565, 95566, 95567, 95568, 95569, 95570, 95571 were selected from the ORFs having the nucleotide sequence represented by SEQ ID NO: 76488.

The probes having the nucleotide sequence represented by SEQ ID NOS: 95572, 95573, 95574, 95575, 95576, 95577, 95578, 95579, 95580, 95581, 95582 were selected from the ORFs having the nucleotide sequence represented by SEQ ID NO: 76489.

The probes having the nucleotide sequence represented by SEQ ID NOS: 95583, 95584, 95585, 95586, 95587, 95588, 95589, 95590, 95591, 95592, 95593 were selected from the ORFs having the nucleotide sequence represented by SEQ ID NO: 76490.

The probes having the nucleotide sequence represented by SEQ ID NOS: 95594, 95595, 95596, 95597, 95598, 95599, 95600, 95601, 95602, 95603, 95604 were selected from the ORFs having the nucleotide sequence represented by SEQ ID NO: 76491.

The probes having the nucleotide sequence represented by SEQ ID NOS: 95605, 95606, 95607, 95608, 95609, 95610, 95611, 95612, 95613, 95614, 95615 were selected from the ORFs having the nucleotide sequence represented by SEQ ID NO: 76492.

The probes having the nucleotide sequence represented by SEQ ID NOS: 95616, 95617, 95618, 95619, 95620, 95621, 95622, 95623, 95624, 95625, 95626 were selected from the ORFs having the nucleotide sequence represented by SEQ ID NO: 76493.

The probes having the nucleotide sequence represented by SEQ ID NOS: 95627, 95628, 95629, 95630, 95631, 95632, 95633, 95634, 95635, 95636, 95637 were selected from the ORFs having the nucleotide sequence represented by SEQ ID NO: 76494.

The probes having the nucleotide sequence represented by SEQ ID NOS: 95638, 95639, 95640, 95641, 95642, 95643, 95644, 95645, 95646, 95647, 95648 were selected from the ORFs having the nucleotide sequence represented by SEQ ID NO: 76495.

The probes having the nucleotide sequence represented by SEQ ID NOS: 95649, 95650, 95651, 95652, 95653, 95654, 95655, 95656, 95657, 95658, 95659 were selected from the ORFs having the nucleotide sequence represented by SEQ ID NO: 76496.

The probes having the nucleotide sequence represented by SEQ ID NOS: 95660, 95661, 95662, 95663, 95664, 95665, 95666, 95667, 95668, 95669, 95670 were selected from the ORFs having the nucleotide sequence represented by SEQ ID NO: 76497.

The probes having the nucleotide sequence represented by SEQ ID NOS: 95671, 95672, 95673, 95674, 95675, 95676, 95677, 95678, 95679, 95680, 95681 were selected from the ORFs having the nucleotide sequence represented by SEQ ID NO: 76498.

The probes having the nucleotide sequence represented by SEQ ID NOS: 95682, 95683, 95684, 95685, 95686, 95687, 95688, 95689, 95690, 95691, 95692 were selected from the ORFs having the nucleotide sequence represented by SEQ ID NO: 76499.

The probes having the nucleotide sequence represented by SEQ ID NOS: 95693, 95694, 95695, 95696, 95697, 95698, 95699, 95700, 95701, 95702, 95703 were selected from the ORFs having the nucleotide sequence represented by SEQ ID NO: 76500.

The probes having the nucleotide sequence represented by SEQ ID NOS: 95704, 95705, 95706, 95707, 95708, 95709, 95710, 95711, 95712, 95713, 95714 were selected from the ORFs having the nucleotide sequence represented by SEQ ID NO: 76501.

The probes having the nucleotide sequence represented by SEQ ID NOS: 95715, 95716, 95717, 95718, 95719, 95720, 95721, 95722, 95723, 95724, 95725 were selected from the ORFs having the nucleotide sequence represented by SEQ ID NO: 76502.

The probes having the nucleotide sequence represented by SEQ ID NOS: 95726, 95727, 95728, 95729, 95730, 95731, 95732, 95733, 95734, 95735, 95736 were selected from the ORFs having the nucleotide sequence represented by SEQ ID NO: 76503.

The probes having the nucleotide sequence represented by SEQ ID NOS: 95737, 95738, 95739, 95740, 95741, 95742, 95743, 95744, 95745, 95746, 95747 were selected from the ORFs having the nucleotide sequence represented by SEQ ID NO: 76504.

The probes having the nucleotide sequence represented by SEQ ID NOS: 95748, 95749, 95750, 95751, 95752, 95753, 95754, 95755, 95756, 95757, 95758 were selected from the ORFs having the nucleotide sequence represented by SEQ ID NO: 76505.

The probes having the nucleotide sequence represented by SEQ ID NOS: 95759, 95760, 95761, 95762, 95763, 95764, 95765, 95766, 95767, 95768, 95769 were selected from the ORFs having the nucleotide sequence represented by SEQ ID NO: 76506.

The probes having the nucleotide sequence represented by SEQ ID NOS: 95770, 95771, 95772, 95773, 95774, 95775, 95776, 95777, 95778, 95779, 95780 were selected from the ORFs having the nucleotide sequence represented by SEQ ID NO: 76507.

The probes having the nucleotide sequence represented by SEQ ID NOS: 95781, 95782, 95783, 95784, 95785, 95786, 95787, 95788, 95789, 95790, 95791 were selected from the ORFs having the nucleotide sequence represented by SEQ ID NO: 76508.

The probes having the nucleotide sequence represented by SEQ ID NOS: 95792, 95793, 95794, 95795, 95796, 95797, 95798, 95799, 95800, 95801, 95802 were selected from the ORFs having the nucleotide sequence represented by SEQ ID NO: 76509.

The probes having the nucleotide sequence represented by SEQ ID NOS: 95803, 95804, 95805, 95806, 95807, 95808, 95809, 95810, 95811, 95812, 95813 were selected from the ORFs having the nucleotide sequence represented by SEQ ID NO: 76510.

The probes having the nucleotide sequence represented by SEQ ID NOS: 95814, 95815, 95816, 95817, 95818, 95819, 95820, 95821, 95822, 95823, 95824 were selected from the ORFs having the nucleotide sequence represented by SEQ ID NO: 76511.

The probes having the nucleotide sequence represented by SEQ ID NOS: 95825, 95826, 95827, 95828, 95829, 95830, 95831, 95832, 95833, 95834, 95835 were selected from the ORFs having the nucleotide sequence represented by SEQ ID NO: 76512.

The probes having the nucleotide sequence represented by SEQ ID NOS: 95836, 95837, 95838, 95839, 95840, 95841, 95842, 95843, 95844, 95845, 95846 were selected from the ORFs having the nucleotide sequence represented by SEQ ID NO: 76513.

The probes having the nucleotide sequence represented by SEQ ID NOS: 95847, 95848, 95849, 95850, 95851, 95852, 95853, 95854, 95855, 95856, 95857 were selected from the ORFs having the nucleotide sequence represented by SEQ ID NO: 76514.

The probes having the nucleotide sequence represented by SEQ ID NOS: 95858, 95859, 95860, 95861, 95862, 95863, 95864, 95865, 95866, 95867, 95868 were selected from the ORFs having the nucleotide sequence represented by SEQ ID NO: 76515.

The probes having the nucleotide sequence represented by SEQ ID NOS: 95869, 95870, 95871, 95872, 95873, 95874, 95875, 95876, 95877, 95878, 95879 were selected from the ORFs having the nucleotide sequence represented by SEQ ID NO: 76516.

The probes having the nucleotide sequence represented by SEQ ID NOS: 95880, 95881, 95882, 95883, 95884, 95885, 95886, 95887, 95888, 95889, 95890 were selected from the ORFs having the nucleotide sequence represented by SEQ ID NO: 76517.

The probes having the nucleotide sequence represented by SEQ ID NOS: 95891, 95892, 95893, 95894, 95895, 95896, 95897, 95898, 95899, 95900, 95901 were selected from the ORFs having the nucleotide sequence represented by SEQ ID NO: 76518.

The probes having the nucleotide sequence represented by SEQ ID NOS: 95902, 95903, 95904, 95905, 95906, 95907, 95908, 95909, 95910, 95911, 95912 were selected from the ORFs having the nucleotide sequence represented by SEQ ID NO: 76519.

The probes having the nucleotide sequence represented by SEQ ID NOS: 95913, 95914, 95915, 95916, 95917, 95918, 95919, 95920, 95921, 95922, 95923 were selected from the ORFs having the nucleotide sequence represented by SEQ ID NO: 76520.

The probes having the nucleotide sequence represented by SEQ ID NOS: 95924, 95925, 95926, 95927, 95928, 95929, 95930, 95931, 95932, 95933, 95934 were selected from the ORFs having the nucleotide sequence represented by SEQ ID NO: 76521.

The probes having the nucleotide sequence represented by SEQ ID NOS: 959.35, 95936, 95937, 95938, 95939, 95940, 95941, 95942, 95943, 95944, 95945 were selected from the ORFs having the nucleotide sequence represented by SEQ ID NO: 76522.

The probes having the nucleotide sequence represented by SEQ ID NOS: 95946, 95947, 95948, 95949, 95950, 95951, 95952, 95953, 95954, 95955, 95956 were selected from the ORFs having the nucleotide sequence represented by SEQ ID NO: 76523.

The probes having the nucleotide sequence represented by SEQ ID NOS: 95957, 95958, 95959, 95960, 95961, 95962, 95963, 95964, 95965, 95966, 95967 were selected from the ORFs having the nucleotide sequence represented by SEQ ID NO: 76524.

The probes having the nucleotide sequence represented by SEQ ID NOS: 95968, 95969, 95970, 95971, 95972, 95973, 95974, 95975, 95976, 95977, 95978 were selected from the ORFs having the nucleotide sequence represented by SEQ ID NO: 76525.

The probes having the nucleotide sequence represented by SEQ ID NOS: 95979, 95980, 95981, 95982, 95983, 95984, 95985, 95986, 95987, 95988, 95989 were selected from the ORFs having the nucleotide sequence represented by SEQ ID NO: 76526.

The probes having the nucleotide sequence represented by SEQ ID NOS: 95990, 95991, 95992, 95993, 95994, 95995, 95996, 95997, 95998, 95999, 96000 were selected from the ORFs having the nucleotide sequence represented by SEQ ID NO: 76527.

The probes having the nucleotide sequence represented by SEQ ID NOS: 96001, 96002, 96003, 96004, 96005, 96006, 96007, 96008, 96009, 96010, 96011 were selected from the ORFs having the nucleotide sequence represented by SEQ ID NO: 76528.

The probes having the nucleotide sequence represented by SEQ ID NOS: 96012, 96013, 96014, 96015, 96016, 96017, 96018, 96019, 96020, 96021, 96022 were selected from the ORFs having the nucleotide sequence represented by SEQ ID NO: 76529.

The probes having the nucleotide sequence represented by SEQ ID NOS: 96023, 96024, 96025, 96026, 96027, 96028, 96029, 96030, 96031, 96032, 96033 were selected from the ORFs having the nucleotide sequence represented by SEQ ID NO: 76530.

The probes having the nucleotide sequence represented by SEQ ID NOS: 96034, 96035, 96036, 96037, 96038, 96039, 96040, 96041, 96042, 96043, 96044 were selected from the ORFs having the nucleotide sequence represented by SEQ ID NO: 76531.

The probes having the nucleotide sequence represented by SEQ ID NOS: 96045, 96046, 96047, 96048, 96049, 96050, 96051, 96052, 96053, 96054, 96055 were selected from the ORFs having the nucleotide sequence represented by SEQ ID NO: 76532.

The probes having the nucleotide sequence represented by SEQ ID NOS: 96056, 96057, 96058, 96059, 96060, 96061, 96062, 96063, 96064, 96065, 96066 were selected from the ORFs having the nucleotide sequence represented by SEQ ID NO: 76533.

The probes having the nucleotide sequence represented by SEQ ID NOS: 96067, 96068, 96069, 96070, 96071, 96072, 96073, 96074, 96075, 96076, 96077 were selected from the ORFs having the nucleotide sequence represented by SEQ ID NO: 76534.

The probes having the nucleotide sequence represented by SEQ ID NOS: 96078, 96079, 96080, 96081, 96082, 96083, 96084, 96085, 96086, 96087, 96088 were selected from the ORFs having the nucleotide sequence represented by SEQ ID NO: 76535.

The probes having the nucleotide sequence represented by SEQ ID NOS: 96089, 96090, 96091, 96092, 96093, 96094, 96095, 96096, 96097, 96098, 96099 were selected from the ORFs having the nucleotide sequence represented by SEQ ID NO: 76536.

The probes having the nucleotide sequence represented by SEQ ID NOS: 96100, 96101, 96102, 96103, 96104, 96105, 96106, 96107, 96108, 96109, 96110 were selected from the ORFs having the nucleotide sequence represented by SEQ ID NO: 76537.

The probes having the nucleotide sequence represented by SEQ ID NOS: 96111, 96112, 96113, 96114, 96115, 96116, 96117, 96118, 96119, 96120, 96121 were selected from the ORFs having the nucleotide sequence represented by SEQ ID NO: 76538.

The probes having the nucleotide sequence represented by SEQ ID NOS: 96122, 96123, 96124, 96125, 96126, 96127, 96128, 96129, 96130, 96131, 96132 were selected from the ORFs having the nucleotide sequence represented by SEQ ID NO: 76539.

The probes having the nucleotide sequence represented by SEQ ID NOS: 96133, 96134, 96135, 96136, 96137, 96138, 96139, 96140, 96141, 96142, 96143 were selected from the ORFs having the nucleotide sequence represented by SEQ ID NO: 76540.

The probes having the nucleotide sequence represented by SEQ ID NOS: 96144, 96145, 96146, 96147, 96148, 96149, 96150, 96151, 96152, 96153, 96154 were selected from the ORFs having the nucleotide sequence represented by SEQ ID NO: 76541.

The probes having the nucleotide sequence represented by SEQ ID NOS: 96155, 96156, 96157, 96158, 96159, 96160, 96161, 96162, 96163, 96164, 96165 were selected from the ORFs having the nucleotide sequence represented by SEQ ID NO: 76542.

The probes having the nucleotide sequence represented by SEQ ID NOS: 96166, 96167, 96168, 96169, 96170, 96171, 96172, 96173, 96174, 96175, 96176 were selected from the ORFs having the nucleotide sequence represented by SEQ ID NO: 76543.

The probes having the nucleotide sequence represented by SEQ ID NOS: 96177, 96178, 96179, 96180, 96181, 96182, 96183, 96184, 96185, 96186, 96187 were selected from the ORFs having the nucleotide sequence represented by SEQ ID NO: 76544.

The probes having the nucleotide sequence represented by SEQ ID NOS: 96188, 96189, 96190, 96191, 96192, 96193, 96194, 96195, 96196, 96197, 96198 were selected from the ORFs having the nucleotide sequence represented by SEQ ID NO: 76545.

The probes having the nucleotide sequence represented by SEQ ID NOS: 96199, 96200, 96201, 96202, 96203, 96204, 96205, 96206, 96207, 96208, 96209 were selected from the ORFs having the nucleotide sequence represented by SEQ ID NO: 76546.

The probes having the nucleotide sequence represented by SEQ ID NOS: 96210, 96211, 96212, 96213, 96214, 96215, 96216, 96217, 96218, 96219, 96220 were selected from the ORFs having the nucleotide sequence represented by SEQ ID NO: 76547.

The probes having the nucleotide sequence represented by SEQ ID NOS: 96221, 96222, 96223, 96224, 96225, 96226, 96227, 96228, 96229, 96230, 96231 were selected from the ORFs having the nucleotide sequence represented by SEQ ID NO: 76548.

The probes having the nucleotide sequence represented by SEQ ID NOS: 96232, 96233, 96234, 96235, 96236, 96237, 96238, 96239, 96240, 96241, 96242 were selected from the ORFs having the nucleotide sequence represented by SEQ ID NO: 76549.

The probes having the nucleotide sequence represented by SEQ ID NOS: 96243, 96244, 96245, 96246, 96247, 96248, 96249, 96250, 96251, 96252, 96253 were selected from the ORFs having the nucleotide sequence represented by SEQ ID NO: 76550.

The probes having the nucleotide sequence represented by SEQ ID NOS: 96254, 96255, 96256, 96257, 96258, 96259, 96260, 96261, 96262, 96263, 96264 were selected from the ORFs having the nucleotide sequence represented by SEQ ID NO: 76551.

The probes having the nucleotide sequence represented by SEQ ID NOS: 96265, 96266, 96267, 96268, 96269, 96270, 96271, 96272, 96273, 96274, 96275 were selected from the ORFs having the nucleotide sequence represented by SEQ ID NO: 76552.

The probes having the nucleotide sequence represented by SEQ ID NOS: 96276, 96277, 96278, 96279, 96280, 96281, 96282, 96283, 96284, 96285, 96286 were selected from the ORFs having the nucleotide sequence represented by SEQ ID NO: 76553.

The probes having the nucleotide sequence represented by SEQ ID NOS: 96287, 96288, 96289, 96290, 96291, 96292, 96293, 96294, 96295, 96296, 96297 were selected from the ORFs having the nucleotide sequence represented by SEQ ID NO: 76554.

The probes having the nucleotide sequence represented by SEQ ID NOS: 96298, 96299, 96300, 96301, 96302, 96303, 96304, 96305, 96306, 96307, 96308 were selected from the ORFs having the nucleotide sequence represented by SEQ ID NO: 76555.

The probes having the nucleotide sequence represented by SEQ ID NOS: 96309, 96310, 96311, 96312, 96313, 96314, 96315, 96316, 96317, 96318, 96319 were selected from the ORFs having the nucleotide sequence represented by SEQ ID NO: 76556.

The probes having the nucleotide sequence represented by SEQ ID NOS: 96320, 96321, 96322, 96323, 96324, 96325, 96326, 96327, 96328, 96329, 96330 were selected from the ORFs having the nucleotide sequence represented by SEQ ID NO: 76557.

The probes having the nucleotide sequence represented by SEQ ID NOS: 96331, 96332, 96333, 96334, 96335, 96336, 96337, 96338, 96339, 96340, 96341 were selected from the ORFs having the nucleotide sequence represented by SEQ ID NO: 76558.

The probes having the nucleotide sequence represented by SEQ ID NOS: 96342, 96343, 96344, 96345, 96346, 96347, 96348, 96349, 96350, 96351, 96352 were selected from the ORFs having the nucleotide sequence represented by SEQ ID NO: 76559.

The probes having the nucleotide sequence represented by SEQ ID NOS: 96353, 96354, 96355, 96356, 96357, 96358, 96359, 96360, 96361, 96362, 96363 were selected from the ORFs having the nucleotide sequence represented by SEQ ID NO: 76560.

The probes having the nucleotide sequence represented by SEQ ID NOS: 96364, 96365, 96366, 96367, 96368, 96369, 96370, 96371, 96372, 96373, 96374 were selected from the ORFs having the nucleotide sequence represented by SEQ ID NO: 76561.

The probes having the nucleotide sequence represented by SEQ ID NOS: 96375, 96376, 96377, 96378, 96379, 96380, 96381, 96382, 96383, 96384, 96385 were selected from the ORFs having the nucleotide sequence represented by SEQ ID NO: 76562.

The probes having the nucleotide sequence represented by SEQ ID NOS: 96386, 96387, 96388, 96389, 96390, 96391, 96392, 96393, 96394, 96395, 96396 were selected from the ORFs having the nucleotide sequence represented by SEQ ID NO: 76563.

The probes having the nucleotide sequence represented by SEQ ID NOS: 96397, 96398, 96399, 96400, 96401, 96402, 96403, 96404, 96405, 96406, 96407 were selected from the ORFs having the nucleotide sequence represented by SEQ ID NO: 76564.

The probes having the nucleotide sequence represented by SEQ ID NOS: 96408, 96409, 96410, 96411, 96412, 96413, 96414, 96415, 96416, 96417, 96418 were selected from the ORFs having the nucleotide sequence represented by SEQ ID NO: 76565.

The probes having the nucleotide sequence represented by SEQ ID NOS: 96419, 96420, 96421, 96422, 96423, 96424, 96425, 96426, 96427, 96428, 96429 were selected from the ORFs having the nucleotide sequence represented by SEQ ID NO: 76566.

The probes having the nucleotide sequence represented by SEQ ID NOS: 96430, 96431, 96432, 96433, 96434, 96435, 96436, 96437, 96438, 96439, 96440 were selected from the ORFs having the nucleotide sequence represented by SEQ ID NO: 76567.

The probes having the nucleotide sequence represented by SEQ ID NOS: 96441, 96442, 96443, 96444, 96445, 96446, 96447, 96448, 96449, 96450, 96451 were selected from the ORFs having the nucleotide sequence represented by SEQ ID NO: 76568.

The probes having the nucleotide sequence represented by SEQ ID NOS: 96452, 96453, 96454, 96455, 96456, 96457, 96458, 96459, 96460, 96461, 96462 were selected from the ORFs having the nucleotide sequence represented by SEQ ID NO: 76569.

The probes having the nucleotide sequence represented by SEQ ID NOS: 96463, 96464, 96465, 96466, 96467, 96468, 96469, 96470, 96471, 96472, 96473 were selected from the ORFs having the nucleotide sequence represented by SEQ ID NO: 76570.

The probes having the nucleotide sequence represented by SEQ ID NOS: 96474, 96475, 96476, 96477, 96478, 96479, 96480, 96481, 96482, 96483, 96484 were selected from the ORFs having the nucleotide sequence represented by SEQ ID NO: 76571.

The probes having the nucleotide sequence represented by SEQ ID NOS: 96485, 96486, 96487, 96488, 96489, 96490, 96491, 96492, 96493, 96494, 96495 were selected from the ORFs having the nucleotide sequence represented by SEQ ID NO: 76572.

The probes having the nucleotide sequence represented by SEQ ID NOS: 96496, 96497, 96498, 96499, 96500, 96501, 96502, 96503, 96504, 96505, 96506 were selected from the ORFs having the nucleotide sequence represented by SEQ ID NO: 76573.

The probes having the nucleotide sequence represented by SEQ ID NOS: 96507, 96508, 96509, 96510, 96511, 96512, 96513, 96514, 96515, 96516, 96517, 96518, 96519, 96520, 96521, 96522, 96523, 96524, 96525, 96526, 96527, 96528 were selected from the ORFs having the nucleotide sequence represented by SEQ ID NO: 76574.

The probes having the nucleotide sequence represented by SEQ ID NOS: 96529, 96530, 96531, 96532, 96533, 96534, 96535, 96536, 96537, 96538, 96539 were selected from the ORFs having the nucleotide sequence represented by SEQ ID NO: 76575.

The probes having the nucleotide sequence represented by SEQ ID NOS: 96540, 96541, 96542, 96543, 96544, 96545, 96546, 96547, 96548, 96549, 96550 were selected from the ORFs having the nucleotide sequence represented by SEQ ID NO: 76576.

The probes having the nucleotide sequence represented by SEQ ID NOS: 96551, 96552, 96553, 96554, 96555, 96556, 96557, 96558, 96559, 96560, 96561 were selected from the ORFs having the nucleotide sequence represented by SEQ ID NO: 76577.

The probes having the nucleotide sequence represented by SEQ ID NOS: 96562, 96563, 96564, 96565, 96566, 96567, 96568, 96569, 96570, 96571, 96572 were selected from the ORFs having the nucleotide sequence represented by SEQ ID NO: 76578.

The probes having the nucleotide sequence represented by SEQ ID NOS: 96573, 96574, 96575, 96576, 96577, 96578, 96579, 96580, 96581, 96582, 96583 were selected from the ORFs having the nucleotide sequence represented by SEQ ID NO: 76579.

The probes having the nucleotide sequence represented by SEQ ID NOS: 96584, 96585, 96586, 96587, 96588, 96589, 96590, 96591, 96592, 96593, 96594 were selected from the ORFs having the nucleotide sequence represented by SEQ ID NO: 76580.

The probes having the nucleotide sequence represented by SEQ ID NOS: 96595, 96596, 96597, 96598, 96599, 96600, 96601, 96602, 96603, 96604, 96605 were selected from the ORFs having the nucleotide sequence represented by SEQ ID NO: 76581.

The probes having the nucleotide sequence represented by SEQ ID NOS: 96606, 96607, 96608, 96609, 96610, 96611, 96612, 96613, 96614, 96615, 96616 were selected from the ORFs having the nucleotide sequence represented by SEQ ID NO: 76582.

The probes having the nucleotide sequence represented by SEQ ID NOS: 96617, 96618, 96619, 96620, 96621, 96622, 96623, 96624, 96625, 96626, 96627 were selected from the ORFs having the nucleotide sequence represented by SEQ ID NO: 76583.

The probes having the nucleotide sequence represented by SEQ ID NOS: 96628, 96629, 96630, 96631, 96632, 96633, 96634, 96635, 96636, 96637, 96638 were selected from the ORFs having the nucleotide sequence represented by SEQ ID NO: 76584.

The probes having the nucleotide sequence represented by SEQ ID NOS: 96639, 96640, 96641, 96642, 96643, 96644, 96645, 96646, 96647, 96648, 96649 were selected from the ORFs having the nucleotide sequence represented by SEQ ID NO: 76585.

The probes having the nucleotide sequence represented by SEQ ID NOS: 96650, 96651, 96652, 96653, 96654, 96655, 96656, 96657, 96658, 96659, 96660 were selected from the ORFs having the nucleotide sequence represented by SEQ ID NO: 76586.

The probes having the nucleotide sequence represented by SEQ ID NOS: 96661, 96662, 96663, 96664, 96665, 96666, 96667, 96668, 96669, 96670, 96671 were selected from the ORFs having the nucleotide sequence represented by SEQ ID NO: 76587.

The probes having the nucleotide sequence represented by SEQ ID NOS: 96672, 96673, 96674, 96675, 96676, 96677, 96678, 96679, 96680, 96681, 96682 were selected from the ORFs having the nucleotide sequence represented by SEQ ID NO: 76588.

The probes having the nucleotide sequence represented by SEQ ID NOS: 96683, 96684, 96685, 96686, 96687, 96688, 96689, 96690, 96691, 96692, 96693 were selected from the ORFs having the nucleotide sequence represented by SEQ ID NO: 76589.

The probes having the nucleotide sequence represented by SEQ ID NOS: 96694, 96695, 96696, 96697, 96698, 96699, 96700, 96701, 96702, 96703, 96704 were selected from the ORFs having the nucleotide sequence represented by SEQ ID NO: 76590.

The probes having the nucleotide sequence represented by SEQ ID NOS: 96705, 96706, 96707, 96708, 96709, 96710, 96711, 96712, 96713, 96714, 96715 were selected from the ORFs having the nucleotide sequence represented by SEQ ID NO: 76591.

The probes having the nucleotide sequence represented by SEQ ID NOS: 96716, 96717, 96718, 96719, 96720, 96721, 96722, 96723, 96724, 96725, 96726 were selected from the ORFs having the nucleotide sequence represented by SEQ ID NO: 76592.

The probes having the nucleotide sequence represented by SEQ ID NOS: 96727, 96728, 96729, 96730, 96731, 96732, 96733, 96734, 96735, 96736, 96737 were selected from the ORFs having the nucleotide sequence represented by SEQ ID NO: 76593.

The probes having the nucleotide sequence represented by SEQ ID NOS: 96738, 96739, 96740, 96741, 96742, 96743, 96744, 96745, 96746, 96747, 96748 were selected from the ORFs having the nucleotide sequence represented by SEQ ID NO: 76594.

The probes having the nucleotide sequence represented by SEQ ID NOS: 96749, 96750, 96751, 96752, 96753, 96754, 96755, 96756, 96757, 96758, 96759 were selected from the ORFs having the nucleotide sequence represented by SEQ ID NO: 76595.

The probes having the nucleotide sequence represented by SEQ ID NOS: 96760, 96761, 96762, 96763, 96764, 96765, 96766, 96767, 96768, 96769, 96770 were selected from the ORFs having the nucleotide sequence represented by SEQ ID NO: 76596.

The probes having the nucleotide sequence represented by SEQ ID NOS: 96771, 96772, 96773, 96774, 96775, 96776, 96777, 96778, 96779, 96780, 96781 were selected from the ORFs having the nucleotide sequence represented by SEQ ID NO: 76597.

The probes having the nucleotide sequence represented by SEQ ID NOS: 96782, 96783, 96784, 96785, 96786, 96787, 96788, 96789, 96790, 96791, 96792 were selected from the ORFs having the nucleotide sequence represented by SEQ ID NO: 76598.

The probes having the nucleotide sequence represented by SEQ ID NOS: 96793, 96794, 96795, 96796, 96797, 96798, 96799, 96800, 96801, 96802, 96803 were selected from the ORFs having the nucleotide sequence represented by SEQ ID NO: 76599.

The probes having the nucleotide sequence represented by SEQ ID NOS: 96804, 96805, 96806, 96807, 96808, 96809, 96810, 96811, 96812, 96813, 96814 were selected from the ORFs having the nucleotide sequence represented by SEQ ID NO: 76600.

The probes having the nucleotide sequence represented by SEQ ID NOS: 96815, 96816, 96817, 96818, 96819, 96820, 96821, 96822, 96823, 96824, 96825 were selected from the ORFs having the nucleotide sequence represented by SEQ ID NO: 76601.

The probes having the nucleotide sequence represented by SEQ ID NOS: 96826, 96827, 96828, 96829, 96830, 96831, 96832, 96833, 96834, 96835, 96836 were selected from the ORFs having the nucleotide sequence represented by SEQ ID NO: 76602.

The probes having the nucleotide sequence represented by SEQ ID NOS: 96837, 96838, 96839, 96840, 96841, 96842, 96843, 96844, 96845, 96846, 96847 were selected from the ORFs having the nucleotide sequence represented by SEQ ID NO: 76603.

The probes having the nucleotide sequence represented by SEQ ID NOS: 96848, 96849, 96850, 96851, 96852, 96853, 96854, 96855, 96856, 96857, 96858 were selected from the ORFs having the nucleotide sequence represented by SEQ ID NO: 76604.

The probes having the nucleotide sequence represented by SEQ ID NOS: 96859, 96860, 96861, 96862, 96863, 96864, 96865, 96866, 96867, 96868, 96869 were selected from the ORFs having the nucleotide sequence represented by SEQ ID NO: 76605.

The probes having the nucleotide sequence represented by SEQ ID NOS: 96870, 96871, 96872, 96873, 96874, 96875, 96876, 96877, 96878, 96879, 96880 were selected from the ORFs having the nucleotide sequence represented by SEQ ID NO: 76606.

The probes having the nucleotide sequence represented by SEQ ID NOS: 96881, 96882, 96883, 96884, 96885, 96886, 96887, 96888, 96889, 96890, 96891 were selected from the ORFs having the nucleotide sequence represented by SEQ ID NO: 76607.

The probes having the nucleotide sequence represented by SEQ ID NOS: 96892, 96893, 96894, 96895, 96896, 96897, 96898, 96899, 96900, 96901, 96902 were selected from the ORFs having the nucleotide sequence represented by SEQ ID NO: 76608.

The probes having the nucleotide sequence represented by SEQ ID NOS: 96903, 96904, 96905, 96906, 96907, 96908, 96909, 96910, 96911, 96912, 96913 were selected from the ORFs having the nucleotide sequence represented by SEQ ID NO: 76609.

The probes having the nucleotide sequence represented by SEQ ID NOS: 96914, 96915, 96916, 96917, 96918, 96919, 96920, 96921, 96922, 96923, 96924 were selected from the ORFs having the nucleotide sequence represented by SEQ ID NO: 76610.

The probes having the nucleotide sequence represented by SEQ ID NOS: 96925, 96926, 96927, 96928, 96929, 96930, 96931, 96932, 96933, 96934, 96935 were selected from the ORFs having the nucleotide sequence represented by SEQ ID NO: 76611.

The probes having the nucleotide sequence represented by SEQ ID NOS: 96936, 96937, 96938, 96939, 96940, 96941, 96942, 96943, 96944, 96945, 96946 were selected from the ORFs having the nucleotide sequence represented by SEQ ID NO: 76612.

The probes having the nucleotide sequence represented by SEQ ID NOS: 96947, 96948, 96949, 96950, 96951, 96952, 96953, 96954, 96955, 96956, 96957 were selected from the ORFs having the nucleotide sequence represented by SEQ ID NO: 76613.

The probes having the nucleotide sequence represented by SEQ ID NOS: 96958, 96959, 96960, 96961, 96962, 96963, 96964, 96965, 96966, 96967, 96968 were selected from the ORFs having the nucleotide sequence represented by SEQ ID NO: 76614.

The probes having the nucleotide sequence represented by SEQ ID NOS: 96969, 96970, 96971, 96972, 96973, 96974, 96975, 96976, 96977, 96978, 96979 were selected from the ORFs having the nucleotide sequence represented by SEQ ID NO: 76615.

The probes having the nucleotide sequence represented by SEQ ID NOS: 96980, 96981, 96982, 96983, 96984, 96985, 96986, 96987, 96988, 96989, 96990 were selected from the ORFs having the nucleotide sequence represented by SEQ ID NO: 76616.

The probes having the nucleotide sequence represented by SEQ ID NOS: 96991, 96992, 96993, 96994, 96995, 96996, 96997, 96998, 96999, 97000, 97001 were selected from the ORFs having the nucleotide sequence represented by SEQ ID NO: 76617.

The probes having the nucleotide sequence represented by SEQ ID NOS: 97002, 97003, 97004, 97005, 97006, 97007, 97008, 97009, 97010, 97011, 97012 were selected from the ORFs having the nucleotide sequence-represented by SEQ ID NO: 76618.

The probes having the nucleotide sequence represented by SEQ ID NOS: 97013, 97014, 97015, 97016, 97017, 97018, 97019, 97020, 97021, 97022, 97023 were selected from the ORFs having the nucleotide sequence represented by SEQ ID NO: 76619.

The probes having the nucleotide sequence represented by SEQ ID NOS: 97024, 97025, 97026, 97027, 97028, 97029, 97030, 97031, 97032, 97033, 97034 were selected from the ORFs having the nucleotide sequence represented by SEQ ID NO: 76620.

The probes having the nucleotide sequence represented by SEQ ID NOS: 97035, 97036, 97037, 97038, 97039, 97040, 97041, 97042, 97043, 97044, 97045 were selected from the ORFs having the nucleotide sequence represented by SEQ ID NO: 76621.

The probes having the nucleotide sequence represented by SEQ ID NOS: 97046, 97047, 97048, 97049, 97050, 97051, 97052, 97053, 97054, 97055, 97056 were selected from the ORFs having the nucleotide sequence represented by SEQ ID NO: 76622.

The probes having the nucleotide sequence represented by SEQ ID NOS: 97057, 97058, 97059, 97060, 97061, 97062, 97063, 97064, 97065, 97066, 97067 were selected from the ORFs having the nucleotide sequence represented by SEQ ID NO: 76623.

The probes having the nucleotide sequence represented by SEQ ID NOS: 97068, 97069, 97070, 97071, 97072, 97073, 97074, 97075, 97076, 97077, 97078 were selected from the ORFs having the nucleotide sequence represented by SEQ ID NO: 76624.

The probes having the nucleotide sequence represented by SEQ ID NOS: 97079, 97080, 97081, 97082, 97083, 97084, 97085, 97086, 97087, 97088, 97089 were selected from the ORFs having the nucleotide sequence represented by SEQ ID NO: 76625.

The probes having the nucleotide sequence represented by SEQ ID NOS: 97090, 97091, 97092, 97093, 97094, 97095, 97096, 97097, 97098, 97099, 97100 were selected from the ORFs having the nucleotide sequence represented by SEQ ID NO: 76626.

The probes having the nucleotide sequence represented by SEQ ID NOS: 97101, 97102, 97103, 97104, 97105, 97106, 97107, 97108, 97109, 97110, 97111 were selected from the ORFs having the nucleotide sequence represented by SEQ ID NO: 76627.

The probes having the nucleotide sequence represented by SEQ ID NOS: 97112, 97113, 97114, 97115, 97116, 97117, 97118, 97119, 97120, 97121, 97122 were selected from the ORFs having the nucleotide sequence represented by SEQ ID NO: 76628.

The probes having the nucleotide sequence represented by SEQ ID NOS: 97123, 97124, 97125, 97126, 97127, 97128, 97129, 97130, 97131, 97132, 97133 were selected from the ORFs having the nucleotide sequence represented by SEQ ID NO: 76629.

The probes having the nucleotide sequence represented by SEQ ID NOS: 97134, 97135, 97136, 97137, 97138, 97139, 97140, 97141, 97142, 97143, 97144 were selected from the ORFs having the nucleotide sequence represented by SEQ ID NO: 76630.

The probes having the nucleotide sequence represented by SEQ ID NOS: 97145, 97146, 97147, 97148, 97149, 97150, 97151, 97152, 97153, 97154, 97155 were selected from the ORFs having the nucleotide sequence represented by SEQ ID NO: 76631.

The probes having the nucleotide sequence represented by SEQ ID NOS: 97156, 97157, 97158, 97159, 97160, 97161, 97162, 97163, 97164, 97165, 97166 were selected from the ORFs having the nucleotide sequence represented by SEQ ID NO: 76632.

The probes having the nucleotide sequence represented by SEQ ID NOS: 97167, 97168, 97169, 97170, 97171, 97172, 97173, 97174, 97175, 97176, 97177 were selected from the ORFs having the nucleotide sequence represented by SEQ ID NO: 76633.

The probes having the nucleotide sequence represented by SEQ ID NOS: 97178, 97179, 97180, 97181, 97182, 97183, 97184, 97185, 97186, 97187, 97188 were selected from the ORFs having the nucleotide sequence represented by SEQ ID NO: 76634.

The probes having the nucleotide sequence represented by SEQ ID NOS: 97189, 97190, 97191, 97192, 97193, 97194, 97195, 97196, 97197, 97198, 97199 were selected from the ORFs having the nucleotide sequence represented by SEQ ID NO: 76635.

The probes having the nucleotide sequence represented by SEQ ID NOS: 97200, 97201, 97202, 97203, 97204, 97205, 97206, 97207, 97208, 97209, 97210 were selected from the ORFs having the nucleotide sequence represented by SEQ ID NO: 76636.

The probes having the nucleotide sequence represented by SEQ ID NOS: 97211, 97212, 97213, 97214, 97215, 97216, 97217, 97218, 97219, 97220, 97221 were selected from the ORFs having the nucleotide sequence represented by SEQ ID NO: 76637.

The probes having the nucleotide sequence represented by SEQ ID NOS: 97222, 97223, 97224, 97225, 97226, 97227, 97228, 97229, 97230, 97231, 97232 were selected from the ORFs having the nucleotide sequence represented by SEQ ID NO: 76638.

The probes having the nucleotide sequence represented by SEQ ID NOS: 97233, 97234, 97235, 97236, 97237, 97238, 97239, 97240, 97241, 97242, 97243 were selected from the ORFs having the nucleotide sequence represented by SEQ ID NO: 76639.

The probes having the nucleotide sequence represented by SEQ ID NOS: 97244, 97245, 97246, 97247, 97248, 97249, 97250, 97251, 97252, 97253, 97254 were selected from the ORFs having the nucleotide sequence represented by SEQ ID NO: 76640.

The probes having the nucleotide sequence represented by SEQ ID NOS: 97255, 97256, 97257, 97258, 97259, 97260, 97261, 97262, 97263, 97264, 97265 were selected from the ORFs having the nucleotide sequence represented by SEQ ID NO: 76641.

The probes having the nucleotide sequence represented by SEQ ID NOS: 97266, 97267, 97268, 97269, 97270, 97271, 97272, 97273, 97274, 97275, 97276 were selected from the ORFs having the nucleotide sequence represented by SEQ ID NO: 76642.

The probes having the nucleotide sequence represented by SEQ ID NOS: 97277, 97278, 97279, 97280, 97281, 97282, 97283, 97284, 97285, 97286, 97287 were selected from the ORFs having the nucleotide sequence represented by SEQ ID NO: 76643.

The probes having the nucleotide sequence represented by SEQ ID NOS: 97288, 97289, 97290, 97291, 97292, 97293, 97294, 97295, 97296, 97297, 97298 were selected from the ORFs having the nucleotide sequence represented by SEQ ID NO: 76644.

The probes having the nucleotide sequence represented by SEQ ID NOS: 97299, 97300, 97301, 97302, 97303, 97304, 97305, 97306, 97307, 97308, 97309 were selected from the ORFs having the nucleotide sequence represented by SEQ ID NO: 76645.

The probes having the nucleotide sequence represented by SEQ ID NOS: 97310, 97311, 97312, 97313, 97314, 97315, 97316, 97317, 97318, 97319, 97320 were selected from the ORFs having the nucleotide sequence represented by SEQ ID NO: 76646.

The probes having the nucleotide sequence represented by SEQ ID NOS: 97321, 97322, 97323, 97324, 97325, 97326, 97327, 97328, 97329, 97330, 97331 were selected from the ORFs having the nucleotide sequence represented by SEQ ID NO: 76647.

The probes having the nucleotide sequence represented by SEQ ID NOS: 97332, 97333, 97334, 97335, 97336, 97337, 97338, 97339, 97340, 97341, 97342 were selected from the ORFs having the nucleotide sequence represented by SEQ ID NO: 76648.

The probes having the nucleotide sequence represented by SEQ ID NOS: 97343, 97344, 97345, 97346, 97347, 97348, 97349, 97350, 97351, 97352, 97353 were selected from the ORFs having the nucleotide sequence represented by SEQ ID NO: 76649.

The probes having the nucleotide sequence represented by SEQ ID NOS: 97354, 97355, 97356, 97357, 97358, 97359, 97360, 97361, 97362, 97363, 97364 were selected from the ORFs having the nucleotide sequence represented by SEQ ID NO: 76650.

The probes having the nucleotide sequence represented by SEQ ID NOS: 97365, 97366, 97367, 97368, 97369, 97370, 97371, 97372, 97373, 97374, 97375 were selected from the ORFs having the nucleotide sequence represented by SEQ ID NO: 76651.

The probes having the nucleotide sequence represented by SEQ ID NOS: 97376, 97377, 97378, 97379, 97380, 97381, 97382, 97383, 97384, 97385, 97386 were selected from the ORFs having the nucleotide sequence represented by SEQ ID NO: 76652.

The probes having the nucleotide sequence represented by SEQ ID NOS: 97387, 97388, 97389, 97390, 97391, 97392, 97393, 97394, 97395, 97396, 97397 were selected from the ORFs having the nucleotide sequence represented by SEQ ID NO: 76653.

The probes having the nucleotide sequence represented by SEQ ID NOS: 97398, 97399, 97400, 97401, 97402, 97403, 97404, 97405, 97406, 97407, 97408 were selected from the ORFs having the nucleotide sequence represented by SEQ ID NO: 76654.

The probes having the nucleotide sequence represented by SEQ ID NOS: 97409, 97410, 97411, 97412, 97413, 97414, 97415, 97416, 97417, 97418, 97419 were selected from the ORFs having the nucleotide sequence represented by SEQ ID NO: 76655.

The probes having the nucleotide sequence represented by SEQ ID NOS: 97420, 97421, 97422, 97423, 97424, 97425, 97426, 97427, 97428, 97429, 97430, 97431, 97432, 97433, 97434, 97435, 97436, 97437, 97438, 97439, 97440, 97441 were selected from the ORFs having the nucleotide sequence represented by SEQ ID NO: 76656.

The probes having the nucleotide sequence represented by SEQ ID NOS: 97442, 97443, 97444, 97445, 97446, 97447, 97448, 97449, 97450, 97451, 97452 were selected from the ORFs having the nucleotide sequence represented by SEQ ID NO: 76657.

The probes having the nucleotide sequence represented by SEQ ID NOS: 97453, 97454, 97455, 97456, 97457, 97458, 97459, 97460, 97461, 97462, 97463 were selected from the ORFs having the nucleotide sequence represented by SEQ ID NO: 76658.

The probes having the nucleotide sequence represented by SEQ ID NOS: 97464, 97465, 97466, 97467, 97468, 97469, 97470, 97471, 97472, 97473, 97474 were selected from the ORFs having the nucleotide sequence represented by SEQ ID NO: 76659.

The probes having the nucleotide sequence represented by SEQ ID NOS: 97475, 97476, 97477, 97478, 97479, 97480, 97481, 97482, 97483, 97484, 97485 were selected from the ORFs having the nucleotide sequence represented by SEQ ID NO: 76660.

The probes having the nucleotide sequence represented by SEQ ID NOS: 97486, 97487, 97488, 97489, 97490, 97491, 97492, 97493, 97494, 97495, 97496 were selected from the ORFs having the nucleotide sequence represented by SEQ ID NO: 76661.

The probes having the nucleotide sequence represented by SEQ ID NOS: 97497, 97498, 97499, 97500, 97501, 97502, 97503, 97504, 97505, 97506, 97507 were selected from the ORFs having the nucleotide sequence represented by SEQ ID NO: 76662.

The probes having the nucleotide sequence represented by SEQ ID NOS: 97508, 97509, 97510, 97511, 97512, 97513, 97514, 97515, 97516, 97517, 97518 were selected from the ORFs having the nucleotide sequence represented by SEQ ID NO: 76663.

The probes having the nucleotide sequence represented by SEQ ID NOS: 97519, 97520, 97521, 97522, 97523, 97524, 97525, 97526, 97527, 97528, 97529 were selected from the ORFs having the nucleotide sequence represented by SEQ ID NO: 76664.

The probes having the nucleotide sequence represented by SEQ ID NOS: 97530, 97531, 97532, 97533, 97534, 97535, 97536, 97537, 97538, 97539, 97540 were selected from the ORFs having the nucleotide sequence represented by SEQ ID NO: 76665.

The probes having the nucleotide sequence represented by SEQ ID NOS: 97541, 97542, 97543, 97544, 97545, 97546, 97547, 97548, 97549, 97550, 97551 were selected from the ORFs having the nucleotide sequence represented by SEQ ID NO: 76666.

The probes having the nucleotide sequence represented by SEQ ID NOS: 97552, 97553, 97554, 97555, 97556, 97557, 97558, 97559, 97560, 97561, 97562 were selected from the ORFs having the nucleotide sequence represented by SEQ ID NO: 76667.

The probes having the nucleotide sequence represented by SEQ ID NOS: 97563, 97564, 97565, 97566, 97567, 97568, 97569, 97570, 97571, 97572, 97573 were selected from the ORFs having the nucleotide sequence represented by SEQ ID NO: 76668.

The probes having the nucleotide sequence represented by SEQ ID NOS: 97574, 97575, 97576, 97577, 97578, 97579, 97580, 97581, 97582, 97583, 97584 were selected from the ORFs having the nucleotide sequence represented by SEQ ID NO: 76669.

The probes having the nucleotide sequence represented by. SEQ ID NOS: 97585, 97586, 97587, 97588, 97589, 97590, 97591, 97592, 97593, 97594, 97595 were selected from the ORFs having the nucleotide sequence represented by SEQ ID NO: 76670.

The probes having the nucleotide sequence represented by SEQ ID NOS: 97596, 97597, 97598, 97599, 97600, 97601, 97602, 97603, 97604, 97605, 97606 were selected from the ORFs having the nucleotide sequence represented by SEQ ID NO: 76671.

The probes having the nucleotide sequence represented by SEQ ID NOS: 97607, 97608, 97609, 97610, 97611, 97612, 97613, 97614, 97615, 97616, 97617 were selected from the ORFs having the nucleotide sequence represented by SEQ ID NO: 76672.

The probes having the nucleotide sequence represented by SEQ ID NOS: 97618, 97619, 97620, 97621, 97622, 97623, 97624, 97625, 97626, 97627, 97628 were selected from the ORFs having the nucleotide sequence represented by SEQ ID NO: 76673.

The probes having the nucleotide sequence represented by SEQ ID NOS: 97629, 97630, 97631, 97632, 97633, 97634, 97635, 97636, 97637, 97638, 97639 were selected from the ORFs having the nucleotide sequence represented by SEQ ID NO: 76674.

The probes having the nucleotide sequence represented by SEQ ID NOS: 97640, 97641, 97642, 97643, 97644, 97645, 97646, 97647, 97648, 97649, 97650 were selected from the ORFs having the nucleotide sequence represented by SEQ ID NO: 76675.

The probes having the nucleotide sequence represented by SEQ ID NOS: 97651, 97652, 97653, 97654, 97655, 97656, 97657, 97658, 97659, 97660, 97661 were selected from the ORFs having the nucleotide sequence represented by SEQ ID NO: 76676.

The probes having the nucleotide sequence represented by SEQ ID NOS: 97662, 97663, 97664, 97665, 97666, 97667, 97668, 97669, 97670, 97671, 97672 were selected from the ORFs having the nucleotide sequence represented by SEQ ID NO: 76677.

The probes having the nucleotide sequence represented by SEQ ID NOS: 97673, 97674, 97675, 97676, 97677, 97678, 97679, 97680, 97681, 97682, 97683 were selected from the ORFs having the nucleotide sequence represented by SEQ ID NO: 76678.

The probes having the nucleotide sequence represented by SEQ ID NOS: 97684, 97685, 97686, 97687, 97688, 97689, 97690, 97691, 97692, 97693, 97694 were selected from the ORFs having the nucleotide sequence represented by SEQ ID NO: 76679.

The probes having the nucleotide sequence represented by SEQ ID NOS: 97695, 97696, 97697, 97698, 97699, 97700, 97701, 97702, 97703, 97704, 97705 were selected from the ORFs having the nucleotide sequence represented by SEQ ID NO: 76680.

The probes having the nucleotide sequence represented by SEQ ID NOS: 97706, 97707, 97708, 97709, 97710, 97711, 97712, 97713, 97714, 97715, 97716 were selected from the ORFs having the nucleotide sequence represented by SEQ ID NO: 76681.

The probes having the nucleotide sequence represented by SEQ ID NOS: 97717, 97718, 97719, 97720, 97721, 97722, 97723, 97724, 97725, 97726, 97727 were selected from the ORFs having the nucleotide sequence represented by SEQ ID NO: 76682.

The probes having the nucleotide sequence represented by SEQ ID NOS: 97728, 97729, 97730, 97731, 97732, 97733, 97734, 97735, 97736, 97737, 97738 were selected from the ORFs having the nucleotide sequence represented by SEQ ID NO: 76683.

The probes having the nucleotide sequence represented by SEQ ID NOS: 97739, 97740, 97741, 97742, 97743, 97744, 97745, 97746, 97747, 97748, 97749 were selected from the ORFs having the nucleotide sequence represented by SEQ ID NO: 76684.

The probes having the nucleotide sequence represented by SEQ ID NOS: 97750, 97751, 97752, 97753, 97754, 97755, 97756, 97757, 97758, 97759, 97760 were selected from the ORFs having the nucleotide sequence represented by SEQ ID NO: 76685.

The probes having the nucleotide sequence represented by SEQ ID NOS: 97761, 97762, 97763, 97764, 97765, 97766, 97767, 97768, 97769, 97770, 97771 were selected from the ORFs having the nucleotide sequence represented by SEQ ID NO: 76686.

The probes having the nucleotide sequence represented by SEQ ID NOS: 97772, 97773, 97774, 97775, 97776, 97777, 97778, 97779, 97780, 97781, 97782 were selected from the ORFs having the nucleotide sequence represented by SEQ ID NO: 76687.

The probes having the nucleotide sequence represented by SEQ ID NOS: 97783, 97784, 97785, 97786, 97787, 97788, 97789, 97790, 97791, 97792, 97793 were selected from the ORFs having the nucleotide sequence represented by SEQ ID NO: 76688.

The probes having the nucleotide sequence represented by SEQ ID NOS: 97794, 97795, 97796, 97797, 97798, 97799, 97800, 97801, 97802, 97803, 97804, 97805, 97806, 97807, 97808, 97809, 97810, 97811, 97812, 97813, 97814, 97815 were selected from the ORFs having the nucleotide sequence represented by SEQ ID NO: 76689.

The probes having the nucleotide sequence represented by SEQ ID NOS: 97816, 97817, 97818, 97819, 97820, 97821, 97822, 97823, 97824, 97825, 97826 were selected from the ORFs having the nucleotide sequence represented by SEQ ID NO: 76690.

The probes having the nucleotide sequence represented by SEQ ID NOS: 97827, 97828, 97829, 97830, 97831, 97832, 97833, 97834, 97835, 97836, 97837 were selected from the ORFs having the nucleotide sequence represented by SEQ ID NO: 76691.

The probes having the nucleotide sequence represented by SEQ ID NOS: 97838, 97839, 97840, 97841, 97842, 97843, 97844, 97845, 97846, 97847, 97848 were selected from the ORFs having the nucleotide sequence represented by SEQ ID NO: 76692.

The probes having the nucleotide sequence represented by SEQ ID NOS: 97849, 97850, 97851, 97852, 97853, 97854, 97855, 97856, 97857, 97858, 97859 were selected from the ORFs having the nucleotide sequence represented by SEQ ID NO: 76693.

The probes having the nucleotide sequence represented by SEQ ID NOS: 97860, 97861, 97862, 97863, 97864, 97865, 97866, 97867, 97868, 97869, 97870 were selected from the ORFs having the nucleotide sequence represented by SEQ ID NO: 76694.

The probes having the nucleotide sequence represented by SEQ ID NOS: 97871, 97872, 97873, 97874, 97875, 97876, 97877, 97878, 97879, 97880, 97881 were selected from the ORFs having the nucleotide sequence represented by SEQ ID NO: 76695.

The probes having the nucleotide sequence represented by SEQ ID NOS: 97882, 97883, 97884, 97885, 97886, 97887, 97888, 97889, 97890, 97891, 97892 were selected from the ORFs having the nucleotide sequence represented by SEQ ID NO: 76696.

The probes having the nucleotide sequence represented by SEQ ID NOS: 97893, 97894, 97895, 97896, 97897, 97898, 97899, 97900, 97901, 97902, 97903 were selected from the ORFs having the nucleotide sequence represented by SEQ ID NO: 76697.

The probes having the nucleotide sequence represented by SEQ ID NOS: 97904, 97905, 97906, 97907, 97908, 97909, 97910, 97911, 97912, 97913, 97914 were selected from the ORFs having the nucleotide sequence represented by SEQ ID NO: 76698.

The probes having the nucleotide sequence represented by SEQ ID NOS: 97915, 97916, 97917, 97918, 97919, 97920, 97921, 97922, 97923, 97924, 97925 were selected from the ORFs having the nucleotide sequence represented by SEQ ID NO: 76699.

The probes having the nucleotide sequence represented by SEQ ID NOS: 97926, 97927, 97928, 97929, 97930, 97931, 97932, 97933, 97934, 97935, 97936 were selected from the ORFs having the nucleotide sequence represented by SEQ ID NO: 76700.

The probes having the nucleotide sequence represented by SEQ ID NOS: 97937, 97938, 97939, 97940, 97941, 97942, 97943, 97944, 97945, 97946, 97947 were selected from the ORFs having the nucleotide sequence represented by SEQ ID NO: 76701.

The probes having the nucleotide sequence represented by SEQ ID NOS: 97948, 97949, 97950, 97951, 97952, 97953, 97954, 97955, 97956, 97957, 97958 were selected from the ORFs having the nucleotide sequence represented by SEQ ID NO: 76702.

The probes having the nucleotide sequence represented by SEQ ID NOS: 97959, 9.7960, 97961, 97962, 97963, 97964, 97965, 97966, 97967, 97968, 97969 were selected from the ORFs having the nucleotide sequence represented by SEQ ID NO: 76703.

The probes having the nucleotide sequence represented by SEQ ID NOS: 97970, 97971, 97972, 97973, 97974, 97975, 97976, 97977, 97978, 97979, 97980 were selected from the ORFs having the nucleotide sequence represented by SEQ ID NO: 76704.

The probes having the nucleotide sequence represented by SEQ ID NOS: 97981, 97982, 97983, 97984, 97985, 97986, 97987, 97988, 97989, 97990, 97991 were selected from the ORFs having the nucleotide sequence represented by SEQ ID NO: 76705.

The probes having the nucleotide sequence represented by SEQ ID NOS: 97992, 97993, 97994, 97995, 97996, 97997, 97998, 97999, 98000, 98001, 98002 were selected from the ORFs having the nucleotide sequence represented by SEQ ID NO: 76706.

The probes having the nucleotide sequence represented by SEQ ID NOS: 98003, 98004, 98005, 98006, 98007, 98008, 98009, 98010, 98011, 98012, 98013 were selected from the ORFs having the nucleotide sequence represented by SEQ ID NO: 76707.

The probes having the nucleotide sequence represented by SEQ ID NOS: 98014, 98015, 98016, 98017, 98018, 98019, 98020, 98021, 98022, 98023, 98024 were selected from the ORFs having the nucleotide sequence represented by SEQ ID NO: 76708.

The probes having the nucleotide sequence represented by SEQ ID NOS: 98025, 98026, 98027, 98028, 98029, 98030, 98031, 98032, 98033, 98034, 98035 were selected from the ORFs having the nucleotide sequence represented by SEQ ID NO: 76709.

The probes having the nucleotide sequence represented by SEQ ID NOS: 98036, 98037, 98038, 98039, 98040, 98041, 98042, 98043, 98044, 98045, 98046 were selected from the ORFs having the nucleotide sequence represented by SEQ ID NO: 76710.

The probes having the nucleotide sequence represented by SEQ ID NOS: 98047, 98048, 98049, 98050, 98051, 98052, 98053, 98054, 98055, 98056, 98057 were selected from the ORFs having the nucleotide sequence represented by SEQ ID NO: 76711.

The probes having the nucleotide sequence represented by SEQ ID NOS: 98058, 98059, 98060, 98061, 98062, 98063, 98064, 98065, 98066, 98067, 98068 were selected from the ORFs having the nucleotide sequence represented by SEQ ID NO: 76712.

The probes having the nucleotide sequence represented by SEQ ID NOS: 98069, 98070, 98071, 98072, 98073, 98074, 98075, 98076, 98077, 98078, 98079 were selected from the ORFs having the nucleotide sequence represented by SEQ ID NO: 76713.

The probes having the nucleotide sequence represented by SEQ ID NOS: 98080, 98081, 98082, 98083, 98084, 98085, 98086, 98087, 98088, 98089, 98090 were selected from the ORFs having the nucleotide sequence represented by SEQ ID NO: 76714.

The probes having the nucleotide sequence represented by SEQ ID NOS: 98091, 98092, 98093, 98094, 98095, 98096, 98097, 98098, 98099, 98100, 98101 were selected from the ORFs having the nucleotide sequence represented by SEQ ID NO: 76715.

The probes having the nucleotide sequence represented by SEQ ID NOS: 98102, 98103, 98104, 98105, 98106, 98107, 98108, 98109, 98110, 98111, 98112 were selected from the ORFs having the nucleotide sequence represented by SEQ ID NO: 76716.

The probes having the nucleotide sequence represented by SEQ ID NOS: 98113, 98114, 98115, 98116, 98117, 98118, 98119, 98120, 98121, 98122, 98123 were selected from the ORFs having the nucleotide sequence represented by SEQ ID NO: 76717.

The probes having the nucleotide sequence represented by SEQ ID NOS: 98124, 98125, 98126, 98127, 98128, 98129, 98130, 98131, 98132, 98133, 98134 were selected from the ORFs having the nucleotide sequence represented by SEQ ID NO: 76718.

The probes having the nucleotide sequence represented by SEQ ID NOS:.98135, 98136, 98137, 98138, 98139, 98140, 98141, 98142, 98143, 98144, 98145 were selected from the ORFs having the nucleotide sequence represented by SEQ ID NO: 76719.

The probes having the nucleotide sequence represented by SEQ ID NOS: 98146, 98147, 98148, 98149, 98150, 98151, 98152, 98153, 98154, 98155, 98156 were selected from the ORFs having the nucleotide sequence represented by SEQ ID NO: 76720.

The probes having the nucleotide sequence represented by SEQ ID NOS: 98157, 98158, 98159, 98160, 98161, 98162, 98163, 98164, 98165, 98166, 98167 were selected from the ORFs having the nucleotide sequence represented by SEQ ID NO: 76721.

The probes having the nucleotide sequence represented by SEQ ID NOS: 98168, 98169, 98170, 98171, 98172, 98173, 98174, 98175, 98176, 98177, 98178 were selected from the ORFs having the nucleotide sequence represented by SEQ ID NO: 76722.

The probes having the nucleotide sequence represented by SEQ ID NOS: 98179, 98180, 98181, 98182, 98183, 98184, 98185, 98186, 98187, 98188, 98189 were selected from the ORFs having the nucleotide sequence represented by SEQ ID NO: 76723.

The probes having the nucleotide sequence represented by SEQ ID NOS: 98190, 98191, 98192, 98193, 98194, 98195, 98196, 98197, 98198, 98199, 98200 were selected from the ORFs having the nucleotide sequence represented by SEQ ID NO: 76724.

The probes having the nucleotide sequence represented by SEQ ID NOS: 98201, 98202, 98203, 98204, 98205, 98206, 98207, 98208, 98209, 98210, 98211 were selected from the ORFs having the nucleotide sequence represented by SEQ ID NO: 76725.

The probes having the nucleotide sequence represented by SEQ ID NOS: 98212, 98213, 98214, 98215, 98216, 98217, 98218, 98219, 98220, 98221, 98222 were selected from the ORFs having the nucleotide sequence represented by SEQ ID NO: 76726.

The probes having the nucleotide sequence represented by SEQ ID NOS: 98223, 98224, 98225, 98226, 98227, 98228, 98229, 98230, 98231, 98232, 98233 were selected from the ORFs having the nucleotide sequence represented by SEQ ID NO: 76727.

The probes having the nucleotide sequence represented by SEQ ID NOS: 98234, 98235, 98236, 98237, 98238, 98239, 98240, 98241, 98242, 98243, 98244 were selected from the ORFs having the nucleotide sequence represented by SEQ ID NO: 76728.

The probes having the nucleotide sequence represented by SEQ ID NOS: 98245, 98246, 98247, 98248, 98249, 98250, 98251, 98252, 98253, 98254, 98255 were selected from the ORFs having the nucleotide sequence represented by SEQ ID NO: 76729.

The probes having the nucleotide sequence represented by SEQ ID NOS: 98256, 98257, 98258, 98259, 98260, 98261, 98262, 98263, 98264, 98265, 98266 were selected from the ORFs having the nucleotide sequence represented by SEQ ID NO: 76730.

The probes having the nucleotide sequence represented by SEQ ID NOS: 98267, 98268, 98269, 98270, 98271, 98272, 98273, 98274, 98275, 98276, 98277 were selected from the ORFs having the nucleotide sequence represented by SEQ ID NO: 76731.

The probes having the nucleotide sequence represented by SEQ ID NOS: 98278, 98279, 98280, 98281, 98282, 98283, 98284, 98285, 98286, 98287, 98288 were selected from the ORFs having the nucleotide sequence represented by SEQ ID NO: 76732.

The probes having the nucleotide sequence represented by SEQ ID NOS: 98289, 98290, 98291, 98292, 98293, 98294, 98295, 98296, 98297, 98298, 98299 were selected from the ORFs having the nucleotide sequence represented by SEQ ID NO: 76733.

The probes having the nucleotide sequence represented by SEQ ID NOS: 98300, 98301, 98302, 98303, 98304, 98305, 98306, 98307, 98308, 98309, 98310 were selected from the ORFs having the nucleotide sequence represented by SEQ ID NO: 76734.

The probes having the nucleotide sequence represented by SEQ ID NOS: 98311, 98312, 98313, 98314, 98315, 98316, 98317, 98318, 98319, 98320, 98321 were selected from the ORFs having the nucleotide sequence represented by SEQ ID NO: 76735.

The probes having the nucleotide sequence represented by SEQ ID NOS: 98322, 98323, 98324, 98325, 98326, 98327, 98328, 98329, 98330, 98331, 98332 were selected from the ORFs having the nucleotide sequence represented by SEQ ID NO: 76736.

The probes having the nucleotide sequence represented by SEQ ID NOS: 98333, 98334, 98335, 98336, 98337, 98338, 98339, 98340, 98341, 98342, 98343 were selected from the ORFs having the nucleotide sequence represented by SEQ ID NO: 76737.

The probes having the nucleotide sequence represented by SEQ ID NOS: 98344, 98345, 98346, 98347, 98348, 98349, 98350, 98351, 98352, 98353, 98354 were selected from the ORFs having the nucleotide sequence represented by SEQ ID NO: 76738.

The probes having the nucleotide sequence represented by SEQ ID NOS: 98355, 98356, 98357, 98358, 98359, 98360, 98361, 98362, 98363, 98364, 98365 were selected from the ORFs having the nucleotide sequence represented by SEQ ID NO: 76739.

The probes having the nucleotide sequence represented by SEQ ID NOS: 98366, 98367, 98368, 98369, 98370, 98371, 98372, 98373, 98374, 98375, 98376 were selected from the ORFs having the nucleotide sequence represented by SEQ ID NO: 76740.

The probes having the nucleotide sequence represented by SEQ ID NOS: 98377, 98378, 98379, 98380, 98381, 98382, 98383, 98384, 98385, 98386, 98387 were selected from the ORFs having the nucleotide sequence represented by SEQ ID NO: 76741.

The probes having the nucleotide sequence represented by SEQ ID NOS: 98388, 98389, 98390, 98391, 98392, 98393, 98394, 98395, 98396, 98397, 98398 were selected from the ORFs having the nucleotide sequence represented by SEQ ID NO: 76742.

The probes having the nucleotide sequence represented by SEQ ID NOS: 98399, 98400, 98401, 98402, 98403, 98404, 98405, 98406, 98407, 98408, 98409 were selected from the ORFs having the nucleotide sequence represented by SEQ ID NO: 76743.

The probes having the nucleotide sequence represented by SEQ ID NOS: 98410, 98411, 98412, 98413, 98414, 98415, 98416, 98417, 98418, 98419, 98420 were selected from the ORFs having the nucleotide sequence represented by SEQ ID NO: 76744.

The probes having the nucleotide sequence represented by SEQ ID NOS: 98421, 98422, 98423, 98424, 98425, 98426, 98427, 98428, 98429, 98430, 98431 were selected from the ORFs having the nucleotide sequence represented by SEQ ID NO: 76745.

The probes having the nucleotide sequence represented by SEQ ID NOS: 98432, 98433, 98434, 98435, 98436, 98437, 98438, 98439, 98440, 98441, 98442 were selected from the ORFs having the nucleotide sequence represented by SEQ ID NO: 76746.

The probes having the nucleotide sequence represented by SEQ ID NOS: 98443, 98444, 98445, 98446, 98447, 98448, 98449, 98450, 98451, 98452, 98453 were selected from the ORFs having the nucleotide sequence represented by SEQ ID NO: 76747.

The probes having the nucleotide sequence represented by SEQ ID NOS: 98454, 98455, 98456, 98457, 98458, 98459, 98460, 98461, 98462, 98463, 98464 were selected from the ORFs having the nucleotide sequence represented by SEQ ID NO: 76748.

The probes having the nucleotide sequence represented by SEQ ID NOS: 98465, 98466, 98467, 98468, 98469, 98470, 98471, 98472, 98473, 98474, 98475 were selected from the ORFs having the nucleotide sequence represented by SEQ ID NO: 76749.

The probes having the nucleotide sequence represented by SEQ ID NOS: 98476, 98477, 98478, 98479, 98480, 98481, 98482, 98483, 98484, 98485, 98486 were selected from the ORFs having the nucleotide sequence represented by SEQ ID NO: 76750.

The probes having the nucleotide sequence represented by SEQ ID NOS: 98487, 98488, 98489, 98490, 98491, 98492, 98493, 98494, 98495, 98496, 98497 were selected from the ORFs having the nucleotide sequence represented by SEQ ID NO: 76751.

The probes having the nucleotide sequence represented by SEQ ID NOS: 98498, 98499, 98500, 98501, 98502, 98503, 98504, 98505, 98506, 98507, 98508 were selected from the ORFs having the nucleotide sequence represented by SEQ ID NO: 76752.

The probes having the nucleotide sequence represented by SEQ ID NOS: 98509, 98510, 98511, 98512, 98513, 98514, 98515, 98516, 98517, 98518, 98519 were selected from the ORFs having the nucleotide sequence represented by SEQ ID NO: 76753.

The probes having the nucleotide sequence represented by SEQ ID NOS: 98520, 98521, 98522, 98523, 98524, 98525, 98526, 98527, 98528, 98529, 98530 were selected from the ORFs having the nucleotide sequence represented by SEQ ID NO: 76754.

The probes having the nucleotide sequence represented by SEQ ID NOS: 98531, 98532, 98533, 98534, 98535, 98536, 98537, 98538, 98539, 98540, 98541 were selected from the ORFs having the nucleotide sequence represented by SEQ ID NO: 76755.

The probes having the nucleotide sequence represented by SEQ ID NOS: 98542, 98543, 98544, 98545, 98546, 98547, 98548, 98549, 98550, 98551, 98552 were selected from the ORFs having the nucleotide sequence represented by SEQ ID NO: 76756.

The probes having the nucleotide sequence represented by SEQ ID NOS: 98553, 98554, 98555, 98556, 98557, 98558, 98559, 98560, 98561, 98562, 98563 were selected from the ORFs having the nucleotide sequence represented by SEQ ID NO: 76757.

The probes having the nucleotide sequence represented by SEQ ID NOS: 98564, 98565, 98566, 98567, 98568, 98569, 98570, 98571, 98572, 98573, 98574 were selected from the ORFs having the nucleotide sequence represented by SEQ ID NO: 76758.

The probes having the nucleotide sequence represented by SEQ ID NOS: 98575, 98576, 98577, 98578, 98579, 98580, 98581, 98582, 98583, 98584, 98585 were selected from the ORFs having the nucleotide sequence represented by SEQ ID NO: 76759.

The probes having the nucleotide sequence represented by SEQ ID NOS: 98586, 98587, 98588, 98589, 98590, 98591, 98592, 98593, 98594, 98595, 98596 were selected from the ORFs having the nucleotide sequence represented by SEQ ID NO: 76760.

The probes having the nucleotide sequence represented by SEQ ID NOS: 98597, 98598, 98599, 98600, 98601, 98602, 98603, 98604, 98605, 98606, 98607 were selected from the ORFs having the nucleotide sequence represented by SEQ ID NO: 76761.

The probes having the nucleotide sequence represented by SEQ ID NOS: 98608, 98609, 98610, 98611, 98612, 98613, 98614, 98615, 98616, 98617, 98618 were selected from the ORFs having the nucleotide sequence represented by SEQ ID NO: 76762.

The probes having the nucleotide sequence represented by SEQ ID NOS: 98619, 98620, 98621, 98622, 98623, 98624, 98625, 98626, 98627, 98628, 98629 were selected from the ORFs having the nucleotide sequence represented by SEQ ID NO: 76763.

The probes having the nucleotide sequence represented by SEQ ID NOS: 98630, 98631, 98632, 98633, 98634, 98635, 98636, 98637, 98638, 98639, 98640 were selected from the ORFs having the nucleotide sequence represented by SEQ ID NO: 76764.

The probes having the nucleotide sequence represented by SEQ ID NOS: 98641, 98642, 98643, 98644, 98645, 98646, 98647, 98648, 98649, 98650, 98651 were selected from the ORFs having the nucleotide sequence represented by SEQ ID NO: 76765.

The probes having the nucleotide sequence represented by SEQ ID NOS: 98652, 98653, 98654, 98655, 98656, 98657, 98658, 98659, 98660, 98661, 98662 were selected from the ORFs having the nucleotide sequence represented by SEQ ID NO: 76766.

The probes having the nucleotide sequence represented by SEQ ID NOS: 98663, 98664, 98665, 98666, 98667, 98668, 98669, 98670, 98671, 98672, 98673 were selected from the ORFs having the nucleotide sequence represented by SEQ ID NO: 76767.

The probes having the nucleotide sequence represented by SEQ ID NOS: 98674, 98675, 98676, 98677, 98678, 98679, 98680, 98681, 98682, 98683, 98684 were selected from the ORFs having the nucleotide sequence represented by SEQ ID NO: 76768.

The probes having the nucleotide sequence represented by SEQ ID NOS: 98685, 98686, 98687, 98688, 98689, 98690, 98691, 98692, 98693, 98694, 98695 were selected from the ORFs having the nucleotide sequence represented by SEQ ID NO: 76769.

The probes having the nucleotide sequence represented by SEQ ID NOS: 98696, 98697, 98698, 98699, 98700, 98701, 98702, 98703, 98704, 98705, 98706 were selected from the ORFs having the nucleotide sequence represented by SEQ ID NO: 76770.

The probes having the nucleotide sequence represented by SEQ ID NOS: 98707, 98708, 98709, 98710, 98711, 98712, 98713, 98714, 98715, 98716, 98717 were selected from the ORFs having the nucleotide sequence represented by SEQ ID NO: 76771.

The probes having the nucleotide sequence represented by SEQ ID NOS: 98718, 98719, 98720, 98721, 98722, 98723, 98724, 98725, 98726, 98727, 98728 were selected from the ORFs having the nucleotide sequence represented by SEQ ID NO: 76772.

The probes having the nucleotide sequence represented by SEQ ID NOS: 98729, 98730, 98731, 98732, 98733, 98734, 98735, 98736, 98737, 98738, 98739 were selected from the ORFs having the nucleotide sequence represented by SEQ ID NO: 76773.

The probes having the nucleotide sequence represented by SEQ ID NOS: 98740, 98741, 98742, 98743, 98744, 98745, 98746, 98747, 98748, 98749, 98750 were selected from the ORFs having the nucleotide sequence represented by SEQ ID NO: 76774.

The probes having the nucleotide sequence represented by SEQ ID NOS: 98751, 98752, 98753, 98754, 98755, 98756, 98757, 98758, 98759, 98760, 98761 were selected from the ORFs having the nucleotide sequence represented by SEQ ID NO: 76775.

The probes having the nucleotide sequence represented by SEQ ID NOS: 98762, 98763, 98764, 98765, 98766, 98767, 98768, 98769, 98770, 98771, 98772 were selected from the ORFs having the nucleotide sequence represented by SEQ ID NO: 76776.

The probes having the nucleotide sequence represented by SEQ ID NOS: 98773, 98774, 98775, 98776, 98777, 98778, 98779, 98780, 98781, 98782, 98783 were selected from the ORFs having the nucleotide sequence represented by SEQ ID NO: 76777.

The probes having the nucleotide sequence represented by SEQ ID NOS: 98784, 98785, 98786, 98787, 98788, 98789, 98790, 98791, 98792, 98793, 98794, 98795, 98796, 98797, 98798, 98799, 98800, 98801, 98802, 98803, 98804, 98805 were selected from the ORFs having the nucleotide sequence represented by SEQ ID NO: 76778.

The probes having the nucleotide sequence represented by SEQ ID NOS: 98806, 98807, 98808, 98809, 98810, 98811, 98812, 98813, 98814, 98815, 98816 were selected from the ORFs having the nucleotide sequence represented by SEQ ID NO: 76779.

The probes having the nucleotide sequence represented by SEQ ID NOS: 98817, 98818, 98819, 98820, 98821, 98822, 98823, 98824, 98825, 98826, 98827 were selected from the ORFs having the nucleotide sequence represented by SEQ ID NO: 76780.

The probes having the nucleotide sequence represented by SEQ ID NOS: 98828, 98829, 98830, 98831, 98832, 98833, 98834, 98835, 98836, 98837, 98838 were selected from the ORFs having the nucleotide sequence represented by SEQ ID NO: 76781.

The probes having the nucleotide sequence represented by SEQ ID NOS: 98839, 98840, 98841, 98842, 98843, 98844, 98845, 98846, 98847, 98848, 98849 were selected from the ORFs having the nucleotide sequence represented by SEQ ID NO: 76782.

The probes having the nucleotide sequence represented by SEQ ID NOS: 98850, 98851, 98852, 98853, 98854, 98855, 98856, 98857, 98858, 98859, 98860, 98861, 98862, 98863, 98864, 98865, 98866, 98867, 98868, 98869, 98870, 98871 were selected from the ORFs having the nucleotide sequence represented by SEQ ID NO: 76783.

The probes having the nucleotide sequence represented by SEQ ID NOS: 98872, 98873, 98874, 98875, 98876, 98877, 98878, 98879, 98880, 98881, 98882 were selected from the ORFs having the nucleotide sequence represented by SEQ ID NO: 76784.

The probes having the nucleotide sequence represented by SEQ ID NOS: 98883, 98884, 98885, 98886, 98887, 98888, 98889, 98890, 98891, 98892, 98893 were selected from the ORFs having the nucleotide sequence represented by SEQ ID NO: 76785.

The probes having the nucleotide sequence represented by SEQ ID NOS: 98894, 98895, 98896, 98897, 98898, 98899, 98900, 98901, 98902, 98903, 98904 were selected from the ORFs having the nucleotide sequence represented by SEQ ID NO: 76786.

The probes having the nucleotide sequence represented by SEQ ID NOS: 98905, 98906, 98907, 98908, 98909, 98910, 98911, 98912, 98913, 98914, 98915 were selected from the ORFs having the nucleotide sequence represented by SEQ ID NO: 76787.

The probes having the nucleotide sequence represented by SEQ ID NOS: 98916, 98917, 98918, 98919, 98920, 98921, 98922, 98923, 98924, 98925, 98926 were selected from the ORFs having the nucleotide sequence represented by SEQ ID NO: 76788.

The probes having the nucleotide sequence represented by SEQ ID NOS: 98927, 98928, 98929, 98930, 98931, 98932, 98933, 98934, 98935, 98936, 98937 were selected from the ORFs having the nucleotide sequence represented by SEQ ID NO: 76789.

The probes having the nucleotide sequence represented by SEQ ID NOS: 98938, 98939, 98940, 98941, 98942, 98943, 98944, 98945, 98946, 98947, 98948 were selected from the ORFs having the nucleotide sequence represented by SEQ ID NO: 76790.

The probes having the nucleotide sequence represented by SEQ ID NOS: 98949, 98950, 98951, 98952, 98953, 98954, 98955, 98956, 98957, 98958, 98959 were selected from the ORFs having the nucleotide sequence represented by SEQ ID NO: 76791.

The probes having the nucleotide sequence represented by SEQ ID NOS: 98960, 98961, 98962, 98963, 98964, 98965, 98966, 98967, 98968, 98969, 98970 were selected from the ORFs having the nucleotide sequence represented by SEQ ID NO: 76792.

The probes having the nucleotide sequence represented by SEQ ID NOS: 98971, 98972, 98973, 98974, 98975, 98976, 98977, 98978, 98979, 98980, 98981 were selected from the ORFs having the nucleotide sequence represented by SEQ ID NO: 76793.

The probes having the nucleotide sequence represented by SEQ ID NOS: 98982, 98983, 98984, 98985, 98986, 98987, 98988, 98989, 98990, 98991, 98992 were selected from the ORFs having the nucleotide sequence represented by SEQ ID NO: 76794.

The probes having the nucleotide sequence represented by SEQ ID NOS: 98993, 98994, 98995, 98996, 98997, 98998, 98999, 99000, 99001, 99002, 99003 were selected from the ORFs having the nucleotide sequence represented by SEQ ID NO: 76795.

The probes having the nucleotide sequence represented by SEQ ID NOS: 99004, 99005, 99006, 99007, 99008, 99009, 99010, 99011, 99012, 99013, 99014 were selected from the ORFs having the nucleotide sequence represented by SEQ ID NO: 76796.

The probes having the nucleotide sequence represented by SEQ ID NOS: 99015, 99016, 99017, 99018, 99019, 99020, 99021, 99022, 99023, 99024, 99025 were selected from the ORFs having the nucleotide sequence represented by SEQ ID NO: 76797.

The probes having the nucleotide sequence represented by SEQ ID NOS: 99026, 99027, 99028, 99029, 99030, 99031, 99032, 99033, 99034, 99035, 99036 were selected from the ORFs having the nucleotide sequence represented by SEQ ID NO: 76798.

The probes having the nucleotide sequence represented by SEQ ID NOS: 99037, 99038, 99039, 99040, 99041, 99042, 99043, 99044, 99045, 99046, 99047 were selected from the ORFs having the nucleotide sequence represented by SEQ ID NO: 76799.

The probes having the nucleotide sequence represented by SEQ ID NOS: 99048, 99049, 99050, 99051, 99052, 99053, 99054, 99055, 99056, 99057, 99058 were selected from the ORFs having the nucleotide sequence represented by SEQ ID NO: 76800.

The probes having the nucleotide sequence represented by SEQ ID NOS: 99059, 99060, 99061, 99062, 99063, 99064, 99065, 99066, 99067, 99068, 99069 were selected from the ORFs having the nucleotide sequence represented by SEQ ID NO: 76801.

The probes having the nucleotide sequence represented by SEQ ID NOS: 99070, 99071, 99072, 99073, 99074, 99075, 99076, 99077, 99078, 99079, 99080 were selected from the ORFs having the nucleotide sequence represented by SEQ ID NO: 76802.

The probes having the nucleotide sequence represented by SEQ ID NOS: 99081, 99082, 99083, 99084, 99085, 99086, 99087, 99088, 99089, 99090, 99091 were selected from the ORFs having the nucleotide sequence represented by SEQ ID NO: 76803.

The probes having the nucleotide sequence represented by SEQ ID NOS: 99092, 99093, 99094, 99095, 99096, 99097, 99098, 99099, 99100, 99101, 99102 were selected from the ORFs having the nucleotide sequence represented by SEQ ID NO: 76804.

The probes having the nucleotide sequence represented by SEQ ID NOS: 99103, 99104, 99105, 99106, 99107, 99108, 99109, 99110, 99111, 99112, 99113 were selected from the ORFs having the nucleotide sequence represented by SEQ ID NO: 76805.

The probes having the nucleotide sequence represented by SEQ ID NOS: 99114, 99115, 99116, 99117, 99118, 99119, 99120, 99121, 99122, 99123, 99124 were selected from the ORFs having the nucleotide sequence represented by SEQ ID NO: 76806.

The probes having the nucleotide sequence represented by SEQ ID NOS: 99125, 99126, 99127, 99128, 99129, 99130, 99131, 99132, 99133, 99134, 99135 were selected from the ORFs having the nucleotide sequence represented by SEQ ID NO: 76807.

The probes having the nucleotide sequence represented by SEQ ID NOS: 99136, 99137, 99138, 99139, 99140, 99141, 99142, 99143, 99144, 99145, 99146 were selected from the ORFs having the nucleotide sequence represented by SEQ ID NO: 76808.

The probes having the nucleotide sequence represented by SEQ ID NOS: 99147, 99148, 99149, 99150, 99151, 99152, 99153, 99154, 99155, 99156, 99157 were selected from the ORFs having the nucleotide sequence represented by SEQ ID NO: 76809.

The probes having the nucleotide sequence represented by SEQ ID NOS: 99158, 99159, 99160, 99161, 99162, 99163, 99164, 99165, 99166, 99167, 99168 were selected from the ORFs having the nucleotide sequence represented by SEQ ID NO: 76810.

The probes having the nucleotide sequence represented by SEQ ID NOS: 99169, 99170, 99171, 99172, 99173, 99174, 99175, 99176, 99177, 99178, 99179 were selected from the ORFs having the nucleotide sequence represented by SEQ ID NO: 76811.

The probes having the nucleotide sequence represented by SEQ ID NOS: 99180, 99181, 99182, 99183, 99184, 99185, 99186, 99187, 99188, 99189, 99190 were selected from the ORFs having the nucleotide sequence represented by SEQ ID NO: 76812.

The probes having the nucleotide sequence represented by SEQ ID NOS: 99191, 99192, 99193, 99194, 99195, 99196, 99197, 99198, 99199, 99200, 99201 were selected from the ORFs having the nucleotide sequence represented by SEQ ID NO: 76813.

The probes having the nucleotide sequence represented by SEQ ID NOS: 99202, 99203, 99204, 99205, 99206, 99207, 99208, 99209, 99210, 99211, 99212 were selected from the ORFs having the nucleotide sequence represented by SEQ ID NO: 76814.

The probes having the nucleotide sequence represented by SEQ ID NOS: 99213, 99214, 99215, 99216, 99217, 99218, 99219, 99220, 99221, 99222, 99223 were selected from the ORFs having the nucleotide sequence represented by SEQ ID NO: 76815.

The probes having the nucleotide sequence represented by SEQ ID NOS: 99224, 99225, 99226, 99227, 99228, 99229, 99230, 99231, 99232, 99233, 99234 were selected from the ORFs having the nucleotide sequence represented by SEQ ID NO: 76816.

The probes having the nucleotide sequence represented by SEQ ID NOS: 99235, 99236, 99237, 99238, 99239, 99240, 99241, 99242, 99243, 99244, 99245 were selected from the ORFs having the nucleotide sequence represented by SEQ ID NO: 76817.

The probes having the nucleotide sequence represented by SEQ ID NOS: 99246, 99247, 99248, 99249, 99250, 99251, 99252, 99253, 99254, 99255, 99256 were selected from the ORFs having the nucleotide sequence represented by SEQ ID NO: 76818.

The probes having the nucleotide sequence represented by SEQ ID NOS: 99257, 99258, 99259, 99260, 99261, 99262, 99263, 99264, 99265, 99266, 99267 were selected from the ORFs having the nucleotide sequence represented by SEQ ID NO: 76819.

The probes having the nucleotide sequence represented by SEQ ID NOS: 99268, 99269, 99270, 99271, 99272, 99273, 99274, 99275, 99276, 99277, 99278 were selected from the ORFs having the nucleotide sequence represented by SEQ ID NO: 76820.

The probes having the nucleotide sequence represented by SEQ ID NOS: 99279, 99280, 99281, 99282, 99283, 99284, 99285, 99286, 99287, 99288, 99289 were selected from the ORFs having the nucleotide sequence represented by SEQ ID NO: 76821.

The probes having the nucleotide sequence represented by SEQ ID NOS: 99290, 99291, 99292, 99293, 99294, 99295, 99296, 99297, 99298, 99299, 99300 were selected from the ORFs having the nucleotide sequence represented by SEQ ID NO: 76822.

The probes having the nucleotide sequence represented by SEQ ID NOS: 99301, 99302, 99303, 99304, 99305, 99306, 99307, 99308, 99309, 99310, 99311 were selected from the ORFs having the nucleotide sequence represented by SEQ ID NO: 76823.

The probes having the nucleotide sequence represented by SEQ ID NOS: 99312, 99313, 99314, 99315, 99316, 99317, 99318, 99319, 99320, 99321, 99322 were selected from the ORFs having the nucleotide sequence represented by SEQ ID NO: 76824.

The probes having the nucleotide sequence represented by SEQ ID NOS: 99323, 99324, 99325, 99326, 99327, 99328, 99329, 99330, 99331, 99332, 99333 were selected from the ORFs having the nucleotide sequence represented by SEQ ID NO: 76825.

The probes having the nucleotide sequence represented by SEQ ID NOS: 99334, 99335, 99336, 99337, 99338, 99339, 99340, 99341, 99342, 99343, 99344 were selected from the ORFs having the nucleotide sequence represented by SEQ ID NO: 76826.

The probes having the nucleotide sequence represented by SEQ ID NOS: 99345, 99346, 99347, 99348, 99349, 99350, 99351, 99352, 99353, 99354, 99355 were selected from the ORFs having the nucleotide sequence represented by SEQ ID NO: 76827.

The probes having the nucleotide sequence represented by SEQ ID NOS: 99356, 99357, 99358, 99359, 99360, 99361, 99362, 99363, 99364, 99365, 99366 were selected from the ORFs having the nucleotide sequence represented by SEQ ID NO: 76828.

The probes having the nucleotide sequence represented by SEQ ID NOS: 99367, 99368, 99369, 99370, 99371, 99372, 99373, 99374, 99375, 99376, 99377 were selected from the ORFs having the nucleotide sequence represented by SEQ ID NO: 76829.

The probes having the nucleotide sequence represented by SEQ ID NOS: 99378, 99379, 99380, 99381, 99382, 99383, 99384, 99385, 99386, 99387, 99388 were selected from the ORFs having the nucleotide sequence represented by SEQ ID NO: 76830.

The probes having the nucleotide sequence represented by SEQ ID NOS: 99389, 99390, 99391, 99392, 99393, 99394, 99395, 99396, 99397, 99398, 99399 were selected from the ORFs having the nucleotide sequence represented by SEQ ID NO: 76831.

The probes having the nucleotide sequence represented by SEQ ID NOS: 99400, 99401, 99402, 99403, 99404, 99405, 99406, 99407, 99408, 99409, 99410 were selected from the ORFs having the nucleotide sequence represented by SEQ ID NO: 76832.

The probes having the nucleotide sequence represented by SEQ ID NOS: 99411, 99412, 99413, 99414, 99415, 99416, 99417, 99418, 99419, 99420, 99421 were selected from the ORFs having the nucleotide sequence represented by SEQ ID NO: 76833.

The probes having the nucleotide sequence represented by SEQ ID NOS: 99422, 99423, 99424, 99425, 99426, 99427, 99428, 99429, 99430, 99431, 99432 were selected from the ORFs having the nucleotide sequence represented by SEQ ID NO: 76834.

The probes having the nucleotide sequence represented by SEQ ID NOS: 9.9433, 99434, 99435, 99436, 99437, 99438, 99439, 99440, 99441, 99442, 99443 were selected from the ORFs having the nucleotide sequence represented by SEQ ID NO: 76835.

The probes having the nucleotide sequence represented by SEQ ID NOS: 99444, 99445, 99446, 99447, 99448, 99449, 99450, 99451, 99452, 99453, 99454 were selected from the ORFs having the nucleotide sequence represented by SEQ ID NO: 76836.

The probes having the nucleotide sequence represented by SEQ ID NOS: 99455, 99456, 99457, 99458, 99459, 99460, 99461, 99462, 99463, 99464, 99465 were selected from the ORFs having the nucleotide sequence represented by SEQ ID NO: 76837.

The probes having the nucleotide sequence represented by SEQ ID NOS: 99466, 99467, 99468, 99469, 99470, 99471, 99472, 99473, 99474, 99475, 99476 were selected from the ORFs having the nucleotide sequence represented by SEQ ID NO: 76838.

The probes having the nucleotide sequence represented by SEQ ID NOS: 99477, 99478, 99479, 99480, 99481, 99482, 99483, 99484, 99485, 99486, 99487 were selected from the ORFs having the nucleotide sequence represented by SEQ ID NO: 76839.

The probes having the nucleotide sequence represented by SEQ ID NOS: 99488, 99489, 99490, 99491, 99492, 99493, 99494, 99495, 99496, 99497, 99498 were selected from the ORFs having the nucleotide sequence represented by SEQ ID NO: 76840.

The probes having the nucleotide sequence represented by SEQ ID NOS: 99499, 99500, 99501, 99502, 99503, 99504, 99505, 99506, 99507, 99508, 99509 were selected from the ORFs having the nucleotide sequence represented by SEQ ID NO: 76841.

The probes having the nucleotide sequence represented by SEQ ID NOS: 99510, 99511, 99512, 99513, 99514, 99515, 99516, 99517, 99518, 99519, 99520 were selected from the ORFs having the nucleotide sequence represented by SEQ ID NO: 76842.

The probes having the nucleotide sequence represented by SEQ ID NOS: 99521, 99522, 99523, 99524, 99525, 99526, 99527, 99528, 99529, 99530, 99531 were selected from the ORFs having the nucleotide sequence represented by SEQ ID NO: 76843.

The probes having the nucleotide sequence represented by SEQ ID NOS: 99532, 99533, 99534, 99535, 99536, 99537, 99538, 99539, 99540, 99541, 99542 were selected from the ORFs having the nucleotide sequence represented by SEQ ID NO: 76844.

The probes having the nucleotide sequence represented by SEQ ID NOS: 99543, 99544, 99545, 99546, 99547, 99548, 99549, 99550, 99551, 99552, 99553 were selected from the ORFs having the nucleotide sequence represented by SEQ ID NO: 76845.

The probes having the nucleotide sequence represented by SEQ ID NOS: 99554, 99555, 99556, 99557, 99558, 99559, 99560, 99561, 99562, 99563, 99564 were selected from the ORFs having the nucleotide sequence represented by SEQ ID NO: 76846.

The probes having the nucleotide sequence represented by SEQ ID NOS: 99565, 99566, 99567, 99568, 99569, 99570, 99571, 99572, 99573, 99574, 99575 were selected from the ORFs having the nucleotide sequence represented by SEQ ID NO: 76847.

The probes having the nucleotide sequence represented by SEQ ID NOS: 99576, 99577, 99578, 99579, 99580, 99581, 99582, 99583, 99584, 99585, 99586 were selected from the ORFs having the nucleotide sequence represented by SEQ ID NO: 76848.

The probes having the nucleotide sequence represented by SEQ ID NOS: 99587, 99588, 99589, 99590, 99591, 99592, 99593, 99594, 99595, 99596, 99597 were selected from the ORFs having the nucleotide sequence represented by SEQ ID NO: 76849.

The probes having the nucleotide sequence represented by SEQ ID NOS: 99598, 99599, 99600, 99601, 99602, 99603, 99604, 99605, 99606, 99607, 99608 were selected from the ORFs having the nucleotide sequence represented by SEQ ID NO: 76850.

The probes having the nucleotide sequence represented by SEQ ID NOS: 99609, 99610, 99611, 99612, 99613, 99614, 99615, 99616, 99617, 99618, 99619 were selected from the ORFs having the nucleotide sequence represented by SEQ ID NO: 76851.

The probes having the nucleotide sequence represented by SEQ ID NOS: 99620, 99621, 99622, 99623, 99624, 99625, 99626, 99627, 99628, 99629, 99630 were selected from the ORFs having the nucleotide sequence represented by SEQ ID NO: 76852.

The probes having the nucleotide sequence represented by SEQ ID NOS: 99631, 99632, 99633, 99634, 99635, 99636, 99637, 99638, 99639, 99640, 99641 were selected from the ORFs having the nucleotide sequence represented by SEQ ID NO: 76853.

The probes having the nucleotide sequence represented by SEQ ID NOS: 99642, 99643, 99644, 99645, 99646, 99647, 99648, 99649, 99650, 99651, 99652 were selected from the ORFs having the nucleotide sequence represented by SEQ ID NO: 76854.

The probes having the nucleotide sequence represented by SEQ ID NOS: 99653, 99654, 99655, 99656, 99657, 99658, 99659, 99660, 99661, 99662, 99663 were selected from the ORFs having the nucleotide sequence represented by SEQ ID NO: 76855.

The probes having the nucleotide sequence represented by SEQ ID NOS: 99664, 99665, 99666, 99667, 99668, 99669, 99670, 99671, 99672, 99673, 99674 were selected from the ORFs having the nucleotide sequence represented by SEQ ID NO: 76856.

The probes having the nucleotide sequence represented by SEQ ID NOS: 99675, 99676, 99677, 99678, 99679, 99680, 99681, 99682, 99683, 99684, 99685 were selected from the ORFs having the nucleotide sequence represented by SEQ ID NO: 76857.

The probes having the nucleotide sequence represented by SEQ ID NOS: 99686, 99687, 99688, 99689, 99690, 99691, 99692, 99693, 99694, 99695, 99696 were selected from the ORFs having the nucleotide sequence represented by SEQ ID NO: 76858.

The probes having the nucleotide sequence represented by SEQ ID NOS: 99697, 99698, 99699, 99700, 99701, 99702, 99703, 99704, 99705, 99706, 99707 were selected from the ORFs having the nucleotide sequence represented by SEQ ID NO: 76859.

The probes having the nucleotide sequence represented by SEQ ID NOS: 99708, 99709, 99710, 99711, 99712, 99713, 99714, 99715, 99716, 99717, 99718 were selected from the ORFs having the nucleotide sequence represented by SEQ ID NO: 76860.

The probes having the nucleotide sequence represented by SEQ ID NOS: 99719, 99720, 99721, 99722, 99723, 99724, 99725, 99726, 99727, 99728, 99729 were selected from the ORFs having the nucleotide sequence represented by SEQ ID NO: 76861.

The probes having the nucleotide sequence represented by SEQ ID NOS: 99730, 99731, 99732, 99733, 99734, 99735, 99736, 99737, 99738, 99739, 99740 were selected from the ORFs having the nucleotide sequence represented by SEQ ID NO: 76862.

The probes having the nucleotide sequence represented by SEQ ID NOS: 99741, 99742, 99743, 99744, 99745, 99746, 99747, 99748, 99749, 99750, 99751 were selected from the ORFs having the nucleotide sequence represented by SEQ ID NO: 76863.

The probes having the nucleotide sequence represented by SEQ ID NOS: 99752, 99753, 99754, 99755, 99756, 99757, 99758, 99759, 99760, 99761, 99762 were selected from the ORFs having the nucleotide sequence represented by SEQ ID NO: 76864.

The probes having the nucleotide sequence represented by SEQ ID NOS: 99763, 99764, 99765, 99766, 99767, 99768, 99769, 99770, 99771, 99772, 99773 were selected from the ORFs having the nucleotide sequence represented by SEQ ID NO: 76865.

The probes having the nucleotide sequence represented by SEQ ID NOS: 99774, 99775, 99776, 99777, 99778, 99779, 99780, 99781, 99782, 99783, 99784 were selected from the ORFs having the nucleotide sequence represented by SEQ ID NO: 76866.

The probes having the nucleotide sequence represented by SEQ ID NOS: 99785, 99786, 99787, 99788, 99789, 99790, 99791, 99792, 99793, 99794, 99795 were selected from the ORFs having the nucleotide sequence represented by SEQ ID NO: 76867.

The probes having the nucleotide sequence represented by SEQ ID NOS: 99796, 99797, 99798, 99799, 99800, 99801, 99802, 99803, 99804, 99805, 99806 were selected from the ORFs having the nucleotide sequence represented by SEQ ID NO: 76868.

The probes having the nucleotide sequence represented by SEQ ID NOS: 99807, 99808, 99809, 99810, 99811, 99812, 99813, 99814, 99815, 99816, 99817 were selected from the ORFs having the nucleotide sequence represented by SEQ ID NO: 76869.

The probes having the nucleotide sequence represented by SEQ ID NOS: 99818, 99819, 99820, 99821, 99822, 99823, 99824, 99825, 99826, 99827, 99828 were selected from the ORFs having the nucleotide sequence represented by SEQ ID NO: 76870.

The probes having the nucleotide sequence represented by SEQ ID NOS: 99829, 99830, 99831, 99832, 99833, 99834, 99835, 99836, 99837, 99838, 99839 were selected from the ORFs having the nucleotide sequence represented by SEQ ID NO: 76871.

The probes having the nucleotide sequence represented by SEQ ID NOS: 99840, 99841, 99842, 99843, 99844, 99845, 99846, 99847, 99848, 99849, 99850 were selected from the ORFs having the nucleotide sequence represented by SEQ ID NO: 76872.

The probes having the nucleotide sequence represented by SEQ ID NOS: 99851, 99852, 99853, 99854, 99855, 99856, 99857, 99858, 99859, 99860, 99861 were selected from the ORFs having the nucleotide sequence represented by SEQ ID NO: 76873.

The probes having the nucleotide sequence represented by SEQ ID NOS: 99862, 99863, 99864, 99865, 99866, 99867, 99868, 99869, 99870, 99871, 99872 were selected from the ORFs having the nucleotide sequence represented by SEQ ID NO: 76874.

The probes having the nucleotide sequence represented by SEQ ID NOS: 99873, 99874, 99875, 99876, 99877, 99878, 99879, 99880, 99881, 99882, 99883 were selected from the ORFs having the nucleotide sequence represented by SEQ ID NO: 76875.

The probes having the nucleotide sequence represented by SEQ ID NOS: 99884, 99885, 99886, 99887, 99888, 99889, 99890, 99891, 99892, 99893, 99894 were selected from the ORFs having the nucleotide sequence represented by SEQ ID NO: 76876.

The probes having the nucleotide sequence represented by SEQ ID NOS: 99895, 99896, 99897, 99898, 99899, 99900, 99901, 99902, 99903, 99904, 99905 were selected from the ORFs having the nucleotide sequence represented by SEQ ID NO: 76877.

The probes having the nucleotide sequence represented by SEQ ID NOS: 99906, 99907, 99908, 99909, 99910, 99911, 99912, 99913, 99914, 99915, 99916 were selected from the ORFs having the nucleotide sequence represented by SEQ ID NO: 76878.

The probes having the nucleotide sequence represented by SEQ ID NOS: 99917, 99918, 99919, 99920, 99921, 99922, 99923, 99924, 99925, 99926, 99927 were selected from the ORFs having the nucleotide sequence represented by SEQ ID NO: 76879.

The probes having the nucleotide sequence represented by SEQ ID NOS: 99928, 99929, 99930, 99931, 99932, 99933, 99934, 99935, 99936, 99937, 99938 were selected from the ORFs having the nucleotide sequence represented by SEQ ID NO: 76880.

The probes having the nucleotide sequence represented by SEQ ID NOS: 99939, 99940, 99941, 99942, 99943, 99944, 99945, 99946, 99947, 99948, 99949 were selected from the ORFs having the nucleotide sequence represented by SEQ ID NO: 76881.

The probes having the nucleotide sequence represented by SEQ ID NOS: 99950, 99951, 99952, 99953, 99954, 99955, 99956, 99957, 99958, 99959, 99960 were selected from the ORFs having the nucleotide sequence represented by SEQ ID NO: 76882.

The probes having the nucleotide sequence represented by SEQ ID NOS: 99961, 99962, 99963, 99964, 99965, 99966, 99967, 99968, 99969, 99970, 99971 were selected from the ORFs having the nucleotide sequence represented by SEQ ID NO: 76883.

The probes having the nucleotide sequence represented by SEQ ID NOS: 99972, 99973, 99974, 99975, 99976, 99977, 99978, 99979, 99980, 99981, 99982 were selected from the ORFs having the nucleotide sequence represented by SEQ ID NO: 76884.

The probes having the nucleotide sequence represented by SEQ ID NOS: 99983, 99984, 99985, 99986, 99987, 99988, 99989, 99990, 99991, 99992, 99993 were selected from the ORFs having the nucleotide sequence represented by SEQ ID NO: 76885.

The probes having the nucleotide sequence represented by SEQ ID NOS: 99994, 99995, 99996, 99997, 99998, 99999, 100000, 100001, 100002, 100003, 100004 were selected from the ORFs having the nucleotide sequence represented by SEQ ID NO: 76886.

The probes having the nucleotide sequence represented by SEQ ID NOS: 100005, 100006, 100007, 100008, 100009, 100010, 100011, 100012, 100013, 100014, 100015, 100016, 100017, 100018, 100019, 100020, 100021, 100022, 100023, 100024, 100025, 100026 were selected from the ORFs having the nucleotide sequence represented by SEQ ID NO: 76887.

The probes having the nucleotide sequence represented by SEQ ID NOS: 100027, 100028, 100029, 100030, 100031, 100032, 100033, 100034, 100035, 100036, 100037 were selected from the ORFs having the nucleotide sequence represented by SEQ ID NO: 76888.

The probes having the nucleotide sequence represented by SEQ ID NOS: 100038, 100039, 100040, 100041, 100042, 100043, 100044, 100045, 100046, 100047, 100048 were selected from the ORFs having the nucleotide sequence represented by SEQ ID NO: 76889.

The probes having the nucleotide sequence represented by SEQ ID NOS: 100049, 100050, 100051, 100052, 100053, 100054, 100055, 100056, 100057, 100058, 100059 were selected from the ORFs having the nucleotide sequence represented by SEQ ID NO: 76890.

The probes having the nucleotide sequence represented by SEQ ID NOS: 100060, 100061, 100062, 100063, 100064, 100065, 100066, 100067, 100068, 100069, 100070 were selected from the ORFs having the nucleotide sequence represented by SEQ ID NO: 76891.

The probes having the nucleotide sequence represented by SEQ ID NOS: 100071, 100072, 100073, 100074, 100075, 100076, 100077, 100078, 100079, 100080, 100081 were selected from the ORFs having the nucleotide sequence represented by SEQ ID NO: 76892.

The probes having the nucleotide sequence represented by SEQ ID NOS: 100082, 100083, 100084, 100085, 100086, 100087, 100088, 100089, 100090, 100091, 100092 were selected from the ORFs having the nucleotide sequence represented by SEQ ID NO: 76893.

The probes having the nucleotide sequence represented by SEQ ID NOS: 100093, 100094, 100095, 100096, 100097, 100098, 100099, 100100, 100101, 100102, 100103 were selected from the ORFs having the nucleotide sequence represented by SEQ ID NO: 76894.

The probes having the nucleotide sequence represented by SEQ ID NOS: 100104, 100105, 100106, 100107, 100108, 100109, 100110, 100111, 100112, 100113, 100114 were selected from the ORFs having the nucleotide sequence represented by SEQ ID NO: 76895.

The probes having the nucleotide sequence represented by SEQ ID NOS: 100115, 100116, 100117, 100118, 100119, 100120, 100121, 100122, 100123, 100124, 100125 were selected from the ORFs having the nucleotide sequence represented by SEQ ID NO: 76896.

The probes having the nucleotide sequence represented by SEQ ID NOS: 100126, 100127, 100128, 100129, 100130, 100131, 100132, 100133, 100134, 100135, 100136 were selected from the ORFs having the nucleotide sequence represented by SEQ ID NO: 76897.

The probes having the nucleotide sequence represented by SEQ ID NOS: 100137, 100138, 100139, 100140, 100141, 100142, 100143, 100144, 100145, 100146, 100147 were selected from the ORFs having the nucleotide sequence represented by SEQ ID NO: 76898.

The probes having the nucleotide sequence represented by SEQ ID NOS: 100148, 100149, 100150, 100151, 100152, 100153, 100154, 100155, 100156, 100157, 100158 were selected from the ORFs having the nucleotide sequence represented by SEQ ID NO: 76899.

The probes having the nucleotide sequence represented by SEQ ID NOS: 100159, 100160, 100161, 100162, 100163, 100164, 100165, 100166, 100167, 100168, 100169, 100170, 100171, 100172, 100173, 100174, 100175, 100176, 100177, 100178, 100179, 100180, 100181, 100182, 100183, 100184, 100185, 100186, 100187, 100188, 100189, 100190, 100191 were selected from the ORFs having the nucleotide sequence represented by SEQ ID NO: 76900.

The probes having the nucleotide sequence represented by SEQ ID NOS: 100192, 100193, 100194, 100195, 100196, 100197, 100198, 100199, 100200, 100201, 100202 were selected from the ORFs having the nucleotide sequence represented by SEQ ID NO: 76901.

The probes having the nucleotide sequence represented by SEQ ID NOS: 100203, 100204, 100205, 100206, 100207, 100208, 100209, 100210, 100211, 100212, 100213 were selected from the ORFs having the nucleotide sequence represented by SEQ ID NO: 76902.

The probes having the nucleotide sequence represented by SEQ ID NOS: 100214, 100215, 100216, 100217, 100218, 100219, 100220, 100221, 100222, 100223, 100224 were selected from the ORFs having the nucleotide sequence represented by SEQ ID NO: 76903.

The probes having the nucleotide sequence represented by SEQ ID NOS: 100225, 100226, 100227, 100228, 100229, 100230, 100231, 100232, 100233, 100234, 100235 were selected from the ORFs having the nucleotide sequence represented by SEQ ID NO: 76904.

The probes having the nucleotide sequence represented by SEQ ID NOS: 100236, 100237, 100238, 100239, 100240, 100241, 100242, 100243, 100244, 100245, 100246 were selected from the ORFs having the nucleotide sequence represented by SEQ ID NO: 76905.

The probes having the nucleotide sequence represented by SEQ ID NOS: 100247, 100248, 100249, 100250, 100251, 100252, 100253, 100254, 100255, 100256, 100257 were selected from the ORFs having the nucleotide sequence represented by SEQ ID NO: 76906.

The probes having the nucleotide sequence represented by SEQ ID NOS: 100258, 100259, 100260, 100261, 100262, 100263, 100264, 100265, 100266, 100267, 100268 were selected from the ORFs having the nucleotide sequence represented by SEQ ID NO: 76907.

The probes having the nucleotide sequence represented by SEQ ID NOS: 100269, 100270, 100271, 100272, 100273, 100274, 100275, 100276, 100277, 100278, 100279 were selected from the ORFs having the nucleotide sequence represented by SEQ ID NO: 76908.

The probes having the nucleotide sequence represented by SEQ ID NOS: 100280, 100281, 100282, 100283, 100284, 100285, 100286, 100287, 100288, 100289, 100290 were selected from the ORFs having the nucleotide sequence represented by SEQ ID NO: 76909.

The probes having the nucleotide sequence represented by SEQ ID NOS: 100291, 100292, 100293, 100294, 100295, 100296, 100297, 100298, 100299, 100300, 100301 were selected from the ORFs having the nucleotide sequence represented by SEQ ID NO: 76910.

The probes having the nucleotide sequence represented by SEQ ID NOS: 100302, 100303, 100304, 100305, 100306, 100307, 100308, 100309, 100310, 100311, 100312 were selected from the ORFs having the nucleotide sequence represented by SEQ ID NO: 76911.

The probes having the nucleotide sequence represented by SEQ ID NOS: 100313, 100314, 100315, 100316, 100317, 100318, 100319, 100320, 100321, 100322, 100323 were selected from the ORFs having the nucleotide sequence represented by SEQ ID NO: 76912.

The probes having the nucleotide sequence represented by SEQ ID NOS: 100324, 100325, 100326, 100327, 100328, 100329, 100330, 100331, 100332, 100333, 100334 were selected from the ORFs having the nucleotide sequence represented by SEQ ID NO: 76913.

The probes having the nucleotide sequence represented by SEQ ID NOS: 100335, 100336, 100337, 100338, 100339, 100340, 100341, 100342, 100343, 100344, 100345 were selected from the ORFs having the nucleotide sequence represented by SEQ ID NO: 76914.

The probes having the nucleotide sequence represented by SEQ ID NOS: 100346, 100347, 100348, 100349, 100350, 100351, 100352, 100353, 100354, 100355, 100356 were selected from the ORFs having the nucleotide sequence represented by SEQ ID NO: 76915.

The probes having the nucleotide sequence represented by SEQ ID NOS: 100357, 100358, 100359, 100360, 100361, 100362, 100363, 100364, 100365, 100366, 100367 were selected from the ORFs having the nucleotide sequence represented by SEQ ID NO: 76916.

The probes having the nucleotide sequence represented by SEQ ID NOS: 100368, 100369, 100370, 100371, 100372, 100373, 100374, 100375, 100376, 100377, 100378 were selected from the ORFs having the nucleotide sequence represented by SEQ ID NO: 76917.

The probes having the nucleotide sequence represented by SEQ ID NOS: 100379, 100380, 100381, 100382, 100383, 100384, 100385, 100386, 100387, 100388, 100389 were selected from the ORFs having the nucleotide sequence represented by SEQ ID NO: 76918.

The probes having the nucleotide sequence represented by SEQ ID NOS: 100390, 100391, 100392, 100393, 100394, 100395, 100396, 100397, 100398, 100399, 100400 were selected from the ORFs having the nucleotide sequence represented by SEQ ID NO: 76919.

The probes having the nucleotide sequence represented by SEQ ID NOS: 100401, 100402, 100403, 100404, 100405, 100406, 100407, 100408, 100409, 100410, 100411 were selected from the ORFs having the nucleotide sequence represented by SEQ ID NO: 76920.

The probes having the nucleotide sequence represented by SEQ ID NOS: 100412, 100413, 100414, 100415, 100416, 100417, 100418, 100419, 100420, 100421, 100422 were selected from the ORFs having the nucleotide sequence represented by SEQ ID NO: 76921.

The probes having the nucleotide sequence represented by SEQ ID NOS: 100423, 100424, 100425, 100426, 100427, 100428, 100429, 100430, 100431, 100432, 100433 were selected from the ORFs having the nucleotide sequence represented by SEQ ID NO: 76922.

The probes having the nucleotide sequence represented by SEQ ID NOS: 100434, 100435, 100436, 100437, 100438, 100439, 100440, 100441, 100442, 100443, 100444 were selected from the ORFs having the nucleotide sequence represented by SEQ ID NO: 76923.

The probes having the nucleotide sequence represented by SEQ ID NOS: 100445, 100446, 100447, 100448, 100449, 100450, 100451, 100452, 100453, 100454, 100455 were selected from the ORFs having the nucleotide sequence represented by SEQ ID NO: 76924.

The probes having the nucleotide sequence represented by SEQ ID NOS: 100456, 100457, 100458, 100459, 100460, 100461, 100462, 100463, 100464, 100465, 100466 were selected from the ORFs having the nucleotide sequence represented by SEQ ID NO: 76925.

The probes having the nucleotide sequence represented by SEQ ID NOS: 100467, 100468, 100469, 100470, 100471, 100472, 100473, 100474, 100475, 100476, 100477 were selected from the ORFs having the nucleotide sequence represented by SEQ ID NO: 76926.

The probes having the nucleotide sequence represented by SEQ ID NOS: 100478, 100479, 100480, 100481, 100482, 100483, 100484, 100485, 100486, 100487, 100488 were selected from the ORFs having the nucleotide sequence represented by SEQ ID NO: 76927.

The probes having the nucleotide sequence represented by SEQ ID NOS: 100489, 100490, 100491, 100492, 100493, 100494, 100495, 100496, 100497, 100498, 100499 were selected from the ORFs having the nucleotide sequence represented by SEQ ID NO: 76928.

The probes having the nucleotide sequence represented by SEQ ID NOS: 100500, 100501, 100502, 100503, 100504, 100505, 100506, 100507, 100508, 100509, 100510 were selected from the ORFs having the nucleotide sequence represented by SEQ ID NO: 76929.

The probes having the nucleotide sequence represented by SEQ ID NOS: 100511, 100512, 100513, 100514, 100515, 100516, 100517, 100518, 100519, 100520, 100521 were selected from the ORFs having the nucleotide sequence represented by SEQ ID NO: 76930.

The probes having the nucleotide sequence represented by SEQ ID NOS: 100522, 100523, 100524, 100525, 100526, 100527, 100528, 100529, 100530, 100531, 100532 were selected from the ORFs having the nucleotide sequence represented by SEQ ID NO: 76931.

The probes having the nucleotide sequence represented by SEQ ID NOS: 100533, 100534, 100535, 100536, 100537, 100538, 100539, 100540, 100541, 100542, 100543 were selected from the ORFs having the nucleotide sequence represented by SEQ ID NO: 76932.

The probes having the nucleotide sequence represented by SEQ ID NOS: 100544, 100545, 100546, 100547, 100548, 100549, 100550, 100551, 100552, 100553, 100554 were selected from the ORFs having the nucleotide sequence represented by SEQ ID NO: 76933.

The probes having the nucleotide sequence represented by SEQ ID NOS: 100555, 100556, 100557, 100558, 100559, 100560, 100561, 100562, 100563, 100564, 100565, 100566, 100567, 100568, 100569, 100570, 100571, 100572, 100573, 100574, 100575, 100576 were selected from the ORFs having the nucleotide sequence represented by SEQ ID NO: 76934.

The probes having the nucleotide sequence represented by SEQ ID NOS: 100577, 100578, 100579, 100580, 100581, 100582, 100583, 100584, 100585, 100586, 100587 were selected from the ORFs having the nucleotide sequence represented by SEQ ID NO: 76935.

The probes having the nucleotide sequence represented by SEQ ID NOS: 100588, 100589, 100590, 100591, 100592, 100593, 100594, 100595, 100596, 100597, 100598 were selected from the ORFs having the nucleotide sequence represented by SEQ ID NO: 76936.

The probes having the nucleotide sequence represented by SEQ ID NOS: 100599, 100600, 100601, 100602, 100603, 100604, 100605, 100606, 100607, 100608, 100609 were selected from the ORFs having the nucleotide sequence represented by SEQ ID NO: 76937.

The probes having the nucleotide sequence represented by SEQ ID NOS: 100610, 100611, 100612, 100613, 100614, 100615, 100616, 100617, 100618, 100619, 100620 were selected from the ORFs having the nucleotide sequence represented by SEQ ID NO: 76938.

The probes having the nucleotide sequence represented by SEQ ID NOS: 100621, 100622, 100623, 100624, 100625, 100626, 100627, 100628, 100629, 100630, 100631 were selected from the ORFs having the nucleotide sequence represented by SEQ ID NO: 76939.

The probes having the nucleotide sequence represented by SEQ ID NOS: 100632, 100633, 100634, 100635, 100636, 100637, 100638, 100639, 100640, 100641, 100642 were selected from the ORFs having the nucleotide sequence represented by SEQ ID NO: 76940.

The probes having the nucleotide sequence represented by SEQ ID NOS: 100643, 100644, 100645, 100646, 100647, 100648, 100649, 100650, 100651, 100652, 100653 were selected from the ORFs having the nucleotide sequence represented by SEQ ID NO: 76941.

The probes having the nucleotide sequence represented by SEQ ID NOS: 100654, 100655, 100656, 100657, 100658, 100659, 100660, 100661, 100662, 100663, 100664 were selected from the ORFs having the nucleotide sequence represented by SEQ ID NO: 76942.

The probes having the nucleotide sequence represented by SEQ ID NOS: 100665, 100666, 100667, 100668, 100669, 100670, 100671, 100672, 100673, 100674, 100675 were selected from the ORFs having the nucleotide sequence represented by SEQ ID NO: 76943.

The probes having the nucleotide sequence represented by SEQ ID NOS: 100676, 100677, 100678, 100679, 100680, 100681, 100682, 100683, 100684, 100685, 100686 were selected from the ORFs having the nucleotide sequence represented by SEQ ID NO: 76944.

The probes having the nucleotide sequence represented by SEQ ID NOS: 100687, 100688, 100689, 100690, 100691, 100692, 100693, 100694, 100695, 100696, 100697 were selected from the ORFs having the nucleotide sequence represented by SEQ ID NO: 76945.

The probes having the nucleotide sequence represented by SEQ ID NOS: 100698, 100699, 100700, 100701, 100702, 100703, 100704, 100705, 100706, 100707, 100708 were selected from the ORFs having the nucleotide sequence represented by SEQ ID NO: 76946.

The probes having the nucleotide sequence represented by SEQ ID NOS: 100709, 100710, 100711, 100712, 100713, 100714, 100715, 100716, 100717, 100718, 100719 were selected from the ORFs having the nucleotide sequence represented by SEQ ID NO: 76947.

The probes having the nucleotide sequence represented by SEQ ID NOS: 100720, 100721, 100722, 100723, 100724, 100725, 100726, 100727, 100728, 100729, 100730 were selected from the ORFs having the nucleotide sequence represented by SEQ ID NO: 76948.

The probes having the nucleotide sequence represented by SEQ ID NOS: 100731, 100732, 100733, 100734, 100735, 100736, 100737, 100738, 100739, 100740, 100741 were selected from the ORFs having the nucleotide sequence represented by SEQ ID NO: 76949.

The probes having the nucleotide sequence represented by SEQ ID NOS: 100742, 100743, 100744, 100745, 100746, 100747, 100748, 100749, 100750, 100751, 100752 were selected from the ORFs having the nucleotide sequence represented by SEQ ID NO: 76950.

The probes having the nucleotide sequence represented by SEQ ID NOS: 100753, 100754, 100755, 100756, 100757, 100758, 100759, 100760, 100761, 100762, 100763 were selected from the ORFs having the nucleotide sequence represented by SEQ ID NO: 76951.

The probes having the nucleotide sequence represented by SEQ ID NOS: 100764, 100765, 100766, 100767, 100768, 100769, 100770, 100771, 100772, 100773, 100774 were selected from the ORFs having the nucleotide sequence represented by SEQ ID NO: 76952.

The probes having the nucleotide sequence represented by SEQ ID NOS: 100775, 100776, 100777, 100778, 100779, 100780, 100781, 100782, 100783, 100784, 100785, 100786, 100787, 100788, 100789, 100790, 100791, 100792, 100793, 100794, 100795, 100796 were selected from the ORFs having the nucleotide sequence represented by SEQ ID NO: 76953.

The probes having the nucleotide sequence represented by SEQ ID NOS: 100797, 100798, 100799, 100800, 100801, 100802, 100803, 100804, 100805, 100806, 100807 were selected from the ORFs having the nucleotide sequence represented by SEQ ID NO: 76954.

The probes having the nucleotide sequence represented by SEQ ID NOS: 100808, 100809, 100810, 100811, 100812, 100813, 100814, 100815, 100816, 100817, 100818 were selected from the ORFs having the nucleotide sequence represented by SEQ ID NO: 76955.

The probes having the nucleotide sequence represented by SEQ ID NOS: 100819, 100820, 100821, 100822, 100823, 100824, 100825, 100826, 100827, 100828, 100829, 100830, 100831, 100832, 100833, 100834, 100835, 100836, 100837, 100838, 100839, 100840 were selected from the ORFs having the nucleotide sequence represented by SEQ ID NO: 76956.

The probes having the nucleotide sequence represented by SEQ ID NOS: 100841, 100842, 100843, 100844, 100845, 100846, 100847, 100848, 100849, 100850, 100851 were selected from the ORFs having the nucleotide sequence represented by SEQ ID NO: 76957.

The probes having the nucleotide sequence represented by SEQ ID NOS: 100852, 100853, 100854, 100855, 100856, 100857, 100858, 100859, 100860, 100861, 100862 were selected from the ORFs having the nucleotide sequence represented by SEQ ID NO: 76958.

The probes having the nucleotide sequence represented by SEQ ID NOS: 100863, 100864, 100865, 100866, 100867, 100868, 100869, 100870, 100871, 100872, 100873 were selected from the ORFs having the nucleotide sequence represented by SEQ ID NO: 76959.

The probes having the nucleotide sequence represented by SEQ ID NOS: 100874, 100875, 100876, 100877, 100878, 100879, 100880, 100881, 100882, 100883, 100884 were selected from the ORFs having the nucleotide sequence represented by SEQ ID NO: 76960.

The probes having the nucleotide sequence represented by SEQ ID NOS: 100885, 100886, 100887, 100888, 100889, 100890, 100891, 100892, 100893, 100894, 100895 were selected from the ORFs having the nucleotide sequence represented by SEQ ID NO: 76961.

The probes having the nucleotide sequence represented by SEQ ID NOS: 100896, 100897, 100898, 100899, 100900, 100901, 100902, 100903, 100904, 100905, 100906 were selected from the ORFs having the nucleotide sequence represented by SEQ ID NO: 76962.

The probes having the nucleotide sequence represented by SEQ ID NOS: 100907, 100908, 100909, 100910, 100911, 100912, 100913, 100914, 100915, 100916, 100917 were selected from the ORFs having the nucleotide sequence represented by SEQ ID NO: 76963.

The probes having the nucleotide sequence represented by SEQ ID NOS: 100918, 100919, 100920, 100921, 100922, 100923, 100924, 100925, 100926, 100927, 100928 were selected from the ORFs having the nucleotide sequence represented by SEQ ID NO: 76964.

The probes having the nucleotide sequence represented by SEQ ID NOS: 100929, 100930, 100931, 100932, 100933, 100934, 100935, 100936, 100937, 100938, 100939 were selected from the ORFs having the nucleotide sequence represented by SEQ ID NO: 76965.

The probes having the nucleotide sequence represented by SEQ ID NOS: 100940, 100941, 100942, 100943, 100944, 100945, 100946, 100947, 100948, 100949, 100950 were selected from the ORFs having the nucleotide sequence represented by SEQ ID NO: 76966.

The probes having the nucleotide sequence represented by SEQ ID NOS: 100951, 100952, 100953, 100954, 100955, 100956, 100957, 100958, 100959, 100960, 100961 were selected from the ORFs having the nucleotide sequence represented by SEQ ID NO: 76967.

The probes having the nucleotide sequence represented by SEQ ID NOS: 100962, 100963, 100964, 100965, 100966, 100967, 100968, 100969, 100970, 100971, 100972 were selected from the ORFs having the nucleotide sequence represented by SEQ ID NO: 76968.

The probes having the nucleotide sequence represented by SEQ ID NOS: 100973, 100974, 100975, 100976, 100977, 100978, 100979, 100980, 100981, 100982, 100983 were selected from the ORFs having the nucleotide sequence represented by SEQ ID NO: 76969.

The probes having the nucleotide sequence represented by SEQ ID NOS: 100984, 100985, 100986, 100987, 100988, 100989, 100990, 100991, 100992, 100993, 100994 were selected from the ORFs having the nucleotide sequence represented by SEQ ID NO: 76970.

The probes having the nucleotide sequence represented by SEQ ID NOS: 100995, 100996, 100997, 100998, 100999, 101000, 101001, 101002, 101003, 101004, 101005 were selected from the ORFs having the nucleotide sequence represented by SEQ ID NO: 76971.

The probes having the nucleotide sequence represented by SEQ ID NOS: 101006, 101007, 101008, 101009, 101010, 101011, 101012, 101013, 101014, 101015, 101016 were selected from the ORFs having the nucleotide sequence represented by SEQ ID NO: 76972.

The probes having the nucleotide sequence represented by SEQ ID NOS: 101017, 101018, 101019, 101020, 101021, 101022, 101023, 101024, 101025, 101026, 101027 were selected from the ORFs having the nucleotide sequence represented by SEQ ID NO: 76973.

The probes having the nucleotide sequence represented by SEQ ID NOS: 101028, 101029, 101030, 101031, 101032, 101033, 101034, 101035, 101036, 101037, 101038 were selected from the ORFs having the nucleotide sequence represented by SEQ ID NO: 76974.

The probes having the nucleotide sequence represented by SEQ ID NOS: 101039, 101040, 101041, 101042, 101043, 101044, 101045, 101046, 101047, 101048, 101049 were selected from the ORFs having the nucleotide sequence represented by SEQ ID NO: 76975.

The probes having the nucleotide sequence represented by SEQ ID NOS: 101050, 101051, 101052, 101053, 101054, 101055, 101056, 101057, 101058, 101059, 101060 were selected from the ORFs having the nucleotide sequence represented by SEQ ID NO: 76976.

The probes having the nucleotide sequence represented by SEQ ID NOS: 101061, 101062, 101063, 101064, 101065, 101066, 101067, 101068, 101069, 101070, 101071 were selected from the ORFs having the nucleotide sequence represented by SEQ ID NO: 76977.

The probes having the nucleotide sequence represented by SEQ ID NOS: 101072, 101073, 101074, 101075, 101076, 101077, 101078, 101079, 101080, 101081, 101082 were selected from the ORFs having the nucleotide sequence represented by SEQ ID NO: 76978.

The probes having the nucleotide sequence represented by SEQ ID NOS: 101083, 101084, 101085, 101086, 101087, 101088, 101089, 101090, 101091, 101092, 101093 were selected from the ORFs having the nucleotide sequence represented by SEQ ID NO: 76979.

The probes having the nucleotide sequence represented by SEQ ID NOS: 101094, 101095, 101096, 101097, 101098, 101099, 101100, 101101, 101102, 101103, 101104 were selected from the ORFs having the nucleotide sequence represented by SEQ ID NO: 76980.

The probes having the nucleotide sequence represented by SEQ ID NOS: 101105, 101106, 101107, 101108, 101109, 101110, 101111, 101112, 101113, 101114, 101115 were selected from the ORFs having the nucleotide sequence represented by SEQ ID NO: 76981.

The probes having the nucleotide sequence represented by SEQ ID NOS: 101116, 101117, 101118, 101119, 101120, 101121, 101122, 101123, 101124, 101125, 101126 were selected from the ORFs having the nucleotide sequence represented by SEQ ID NO: 76982.

The probes having the nucleotide sequence represented by SEQ ID NOS: 101127, 101128, 101129, 101130, 101131, 101132, 101133, 101134, 101135, 101136, 101137 were selected from the ORFs having the nucleotide sequence represented by SEQ ID NO: 76983.

The probes having the nucleotide sequence represented by SEQ ID NOS: 101138, 101139, 101140, 101141, 101142, 101143, 101144, 101145, 101146, 101147, 101148 were selected from the ORFs having the nucleotide sequence represented by SEQ ID NO: 76984.

The probes having the nucleotide sequence represented by SEQ ID NOS: 101149, 101150, 101151, 101152, 101153, 101154, 101155, 101156, 101157, 101158, 101159 were selected from the ORFs having the nucleotide sequence represented by SEQ ID NO: 76985.

The probes having the nucleotide sequence represented by SEQ ID NOS: 101160, 101161, 101162, 101163, 101164, 101165, 101166, 101167, 101168, 101169, 101170 were selected from the ORFs having the nucleotide sequence represented by SEQ ID NO: 76986.

The probes having the nucleotide sequence represented by SEQ ID NOS: 101171, 101172, 101173, 101174, 101175, 101176, 101177, 101178, 101179, 101180, 101181 were selected from the ORFs having the nucleotide sequence represented by SEQ ID NO: 76987.

The probes having the nucleotide sequence represented by SEQ ID NOS: 101182, 101183, 101184, 101185, 101186, 101187, 101188, 101189, 101190, 101191, 101192 were selected from the ORFs having the nucleotide sequence represented by SEQ ID NO: 76988.

The probes having the nucleotide sequence represented by SEQ ID NOS: 101193, 101194, 101195, 101196, 101197, 101198, 101199, 101200, 101201, 101202, 101203 were selected from the ORFs having the nucleotide sequence represented by SEQ ID NO: 76989.

The probes having the nucleotide sequence represented by SEQ ID NOS: 101204, 101205, 101206, 101207, 101208, 101209, 101210, 101211, 101212, 101213, 101214 were selected from the ORFs having the nucleotide sequence represented by SEQ ID NO: 76990.

The probes having the nucleotide sequence represented by SEQ ID NOS: 101215, 101216, 101217, 101218, 101219, 101220, 101221, 101222, 101223, 101224, 101225 were selected from the ORFs having the nucleotide sequence represented by SEQ ID NO: 76991.

The probes having the nucleotide sequence represented by SEQ ID NOS: 101226, 101227, 101228, 101229, 101230, 101231, 101232, 101233, 101234, 101235, 101236 were selected from the ORFs having the nucleotide sequence represented by SEQ ID NO: 76992.

The probes having the nucleotide sequence represented by SEQ ID NOS: 101237, 101238, 101239, 101240, 101241, 101242, 101243, 101244, 101245, 101246, 101247 were selected from the ORFs having the nucleotide sequence represented by SEQ ID NO: 76993.

The probes having the nucleotide sequence represented by SEQ ID NOS: 101248, 101249, 101250, 101251, 101252, 101253, 101254, 101255, 101256, 101257, 101258 were selected from the ORFs having the nucleotide sequence represented by SEQ ID NO: 76994.

The probes having the nucleotide sequence represented by SEQ ID NOS: 101259, 101260, 101261, 101262, 101263, 101264, 101265, 101266, 101267, 101268, 101269 were selected from the ORFs having the nucleotide sequence represented by SEQ ID NO: 76995.

The probes having the nucleotide sequence represented by SEQ ID NOS: 101270, 101271, 101272, 101273, 101274, 101275, 101276, 101277, 101278, 101279, 101280 were selected from the ORFs having the nucleotide sequence represented by SEQ ID NO: 76996.

The probes having the nucleotide sequence represented by SEQ ID NOS: 101281, 101282, 101283, 101284, 101285, 101286, 101287, 101288, 101289, 101290, 101291 were selected from the ORFs having the nucleotide sequence represented by SEQ ID NO: 76997.

The probes having the nucleotide sequence represented by SEQ ID NOS: 101292, 101293, 101294, 101295, 101296, 101297, 101298, 101299, 101300, 101301, 101302 were selected from the ORFs having the nucleotide sequence represented by SEQ ID NO: 76998.

The probes having the nucleotide sequence represented by SEQ ID NOS: 101303, 101304, 101305, 101306, 101307, 101308, 101309, 101310, 101311, 101312, 101313 were selected from the ORFs having the nucleotide sequence represented by SEQ ID NO: 76999.

The probes having the nucleotide sequence represented by SEQ ID NOS: 101314, 101315, 101316, 101317, 101318, 101319, 101320, 101321, 101322, 101323, 101324 were selected from the ORFs having the nucleotide sequence represented by SEQ ID NO: 77000.

The probes having the nucleotide sequence represented by SEQ ID NOS: 101325, 101326, 101327, 101328, 101329, 101330, 101331, 101332, 101333, 101334, 101335 were selected from the ORFs having the nucleotide sequence represented by SEQ ID NO: 77001.

The probes having the nucleotide sequence represented by SEQ ID NOS: 101336, 101337, 101338, 101339, 101340, 101341, 101342, 101343, 101344, 101345, 101346 were selected from the ORFs having the nucleotide sequence represented by SEQ ID NO: 77002.

The probes having the nucleotide sequence represented by SEQ ID NOS: 101347, 101348, 101349, 101350, 101351, 101352, 101353, 101354, 101355, 101356, 101357 were selected from the ORFs having the nucleotide sequence represented by SEQ ID NO: 77003.

The probes having the nucleotide sequence represented by SEQ ID NOS: 101358, 101359, 101360, 101361, 101362, 101363, 101364, 101365, 101366, 101367, 101368 were selected from the ORFs having the nucleotide sequence represented by SEQ ID NO: 77004.

The probes having the nucleotide sequence represented by SEQ ID NOS: 101369, 101370, 101371, 101372, 101373, 101374, 101375, 101376, 101377, 101378, 101379 were selected from the ORFs having the nucleotide sequence represented by SEQ ID NO: 77005.

The probes having the nucleotide sequence represented by SEQ ID NOS: 101380, 101381, 101382, 101383, 101384, 101385, 101386, 101387, 101388, 101389, 101390 were selected from the ORFs having the nucleotide sequence represented by SEQ ID NO: 77006.

The probes having the nucleotide sequence represented by SEQ ID NOS: 101391, 101392, 101393, 101394, 101395, 101396, 101397, 101398, 101399, 101400, 101401 were selected from the ORFs having the nucleotide sequence represented by SEQ ID NO: 77007.

The probes having the nucleotide sequence represented by SEQ ID NOS: 101402, 101403, 101404, 101405, 101406, 101407, 101408, 101409, 101410, 101411, 101412 were selected from the ORFs having the nucleotide sequence represented by SEQ ID NO: 77008.

The probes having the nucleotide sequence represented by SEQ ID NOS: 101413, 101414, 101415, 101416, 101417, 101418, 101419, 101420, 101421, 101422, 101423 were selected from the ORFs having the nucleotide sequence represented by SEQ ID NO: 77009.

The probes having the nucleotide sequence represented by SEQ ID NOS: 101424, 101425, 101426, 101427, 101428, 101429, 101430, 101431, 101432, 101433, 101434 were selected from the ORFs having the nucleotide sequence represented by SEQ ID NO: 77010.

The probes having the nucleotide sequence represented by SEQ ID NOS: 101435, 101436, 101437, 101438, 101439, 101440, 101441, 101442, 101443, 101444, 101445 were selected from the ORFs having the nucleotide sequence represented by SEQ ID NO: 77011.

The probes having the nucleotide sequence represented by SEQ ID NOS: 101446, 101447, 101448, 101449, 101450, 101451, 101452, 101453, 101454, 101455, 101456 were selected from the ORFs having the nucleotide sequence represented by SEQ ID NO: 77012.

The probes having the nucleotide sequence represented by SEQ ID NOS: 101457, 101458, 101459, 101460, 101461, 101462, 101463, 101464, 101465, 101466, 101467 were selected from the ORFs having the nucleotide sequence represented by SEQ ID NO: 77013.

The probes having the nucleotide sequence represented by SEQ ID NOS: 101468, 101469, 101470, 101471, 101472, 101473, 101474, 101475, 101476, 101477, 101478 were selected from the ORFs having the nucleotide sequence represented by SEQ ID NO: 77014.

The probes having the nucleotide sequence represented by SEQ ID NOS: 101479, 101480, 101481, 101482, 101483, 101484, 101485, 101486, 101487, 101488, 101489 were selected from the ORFs having the nucleotide sequence represented by SEQ ID NO: 77015.

The probes having the nucleotide sequence represented by SEQ ID NOS: 101490, 101491, 101492, 101493, 101494, 101495, 101496, 101497, 101498, 101499, 101500 were selected from the ORFs having the nucleotide sequence represented by SEQ ID NO: 77016.

The probes having the nucleotide sequence represented by SEQ ID NOS: 101501, 101502, 101503, 101504, 101505, 101506, 101507, 101508, 101509, 101510, 101511 were selected from the ORFs having the nucleotide sequence represented by SEQ ID NO: 77017.

The probes having the nucleotide sequence represented by SEQ ID NOS: 101512, 101513, 101514, 101515, 101516, 101517, 101518, 101519, 101520, 101521, 101522 were selected from the ORFs having the nucleotide sequence represented by SEQ ID NO: 77018.

The probes having the nucleotide sequence represented by SEQ ID NOS: 101523, 101524, 101525, 101526, 101527, 101528, 101529, 101530, 101531, 101532, 101533 were selected from the ORFs having the nucleotide sequence represented by SEQ ID NO: 77019.

The probes having the nucleotide sequence represented by SEQ ID NOS: 101534, 101535, 101536, 101537, 101538, 101539, 101540, 101541, 101542, 101543, 101544 were selected from the ORFs having the nucleotide sequence represented by SEQ ID NO: 77020.

The probes having the nucleotide sequence represented by SEQ ID NOS: 101545, 101546, 101547, 101548, 101549, 101550, 101551, 101552, 101553, 101554, 101555 were selected from the ORFs having the nucleotide sequence represented by SEQ ID NO: 77021.

The probes having the nucleotide sequence represented by SEQ ID NOS: 101556, 101557, 101558, 101559, 101560, 101561, 101562, 101563, 101564, 101565, 101566 were selected from the ORFs having the nucleotide sequence represented by SEQ ID NO: 77022.

The probes having the nucleotide sequence represented by SEQ ID NOS: 101567, 101568, 101569, 101570, 101571, 101572, 101573, 101574, 101575, 101576, 101577 were selected from the ORFs having the nucleotide sequence represented by SEQ ID NO: 77023.

The probes having the nucleotide sequence represented by SEQ ID NOS: 101578, 101579, 101580, 101581, 101582, 101583, 101584, 101585, 101586, 101587, 101588 were selected from the ORFs having the nucleotide sequence represented by SEQ ID NO: 77024.

The probes having the nucleotide sequence represented by SEQ ID NOS: 101589, 101590, 101591, 101592, 101593, 101594, 101595, 101596, 101597, 101598, 101599 were selected from the ORFs having the nucleotide sequence represented by SEQ ID NO: 77025.

The probes having the nucleotide sequence represented by SEQ ID NOS: 101600, 101601, 101602, 101603, 101604, 101605, 101606, 101607, 101608, 101609, 101610 were selected from the ORFs having the nucleotide sequence represented by SEQ ID NO: 77026.

The probes having the nucleotide sequence represented by SEQ ID NOS: 101611, 101612, 101613, 101614, 101615, 101616, 101617, 101618, 101619, 101620, 101621 were selected from the ORFs having the nucleotide sequence represented by SEQ ID NO: 77027.

The probes having the nucleotide sequence represented by SEQ ID NOS: 101622, 101623, 101624, 101625, 101626, 101627, 101628, 101629, 101.630, 101631, 101632 were selected from the ORFs having the nucleotide sequence represented by SEQ ID NO: 77028.

The probes having the nucleotide sequence represented by SEQ ID NOS: 101633, 101634, 101635, 101636, 101637, 101638, 101639, 101640, 101641, 101642, 101643 were selected from the ORFs having the nucleotide sequence represented by SEQ ID NO: 77029.

The probes having the nucleotide sequence represented by SEQ ID NOS: 101644, 101645, 101646, 101647, 101648, 101649, 101650, 101651, 101652, 101653, 101654 were selected from the ORFs having the nucleotide sequence represented by SEQ ID NO: 77030.

The probes having the nucleotide sequence represented by SEQ ID NOS: 101655, 101656, 101657, 101658, 101659, 101660, 101661, 101662, 101663, 101664, 101665 were selected from the ORFs having the nucleotide sequence represented by SEQ ID NO: 77031.

The probes having the nucleotide sequence represented by SEQ ID NOS: 101666, 101667, 101668, 101669, 101670, 101671, 101672, 101673, 101674, 101675, 101676 were selected from the ORFs having the nucleotide sequence represented by SEQ ID NO: 77032.

The probes having the nucleotide sequence represented by SEQ ID NOS: 101677, 101678, 101679, 101680, 101681, 101682, 101683, 101684, 101685, 101686, 101687 were selected from the ORFs having the nucleotide sequence represented by SEQ ID NO: 77033.

The probes having the nucleotide sequence represented by SEQ ID NOS: 101688, 101689, 101690, 101691, 101692, 101693, 101694, 101695, 101696, 101697, 101698 were selected from the ORFs having the nucleotide sequence represented by SEQ ID NO: 77034.

The probes having the nucleotide sequence represented by SEQ ID NOS: 101699, 101700, 101701, 101702, 101703, 101704, 101705, 101706, 101707, 101708, 101709 were selected from the ORFs having the nucleotide sequence represented by SEQ ID NO: 77035.

The probes having the nucleotide sequence represented by SEQ ID NOS: 101710, 101711, 101712, 101713, 101714, 101715, 101716, 101717, 101718, 101719, 101720 were selected from the ORFs having the nucleotide sequence represented by SEQ ID NO: 77036.

The probes having the nucleotide sequence represented by SEQ ID NOS: 101721, 101722, 101723, 101724, 101725, 101726, 101727, 101728, 101729, 101730, 101731 were selected from the ORFs having the nucleotide sequence represented by SEQ ID NO: 77037.

The probes having the nucleotide sequence represented by SEQ ID NOS: 101732, 101733, 101734, 101735, 101736, 101737, 101738, 101739, 101740, 101741, 101742 were selected from the ORFs having the nucleotide sequence represented by SEQ ID NO: 77038.

The probes having the nucleotide sequence represented by SEQ ID NOS: 101743, 101744, 101745, 101746, 101747, 101748, 101749, 101750, 101751, 101752, 101753 were selected from the ORFs having the nucleotide sequence represented by SEQ ID NO: 77039.

The probes having the nucleotide sequence represented by SEQ ID NOS: 101754, 101755, 101756, 101757, 101758, 101759, 101760, 101761, 101762, 101763, 101764 were selected from the ORFs having the nucleotide sequence represented by SEQ ID NO: 77040.

The probes having the nucleotide sequence represented by SEQ ID NOS: 101765, 101766, 101767, 101768, 101769, 101770, 101771, 101772, 101773, 101774, 101775 were selected from the ORFs having the nucleotide sequence represented by SEQ ID NO: 77041.

The probes having the nucleotide sequence represented by SEQ ID NOS: 101776, 101777, 101778, 101779, 101780, 101781, 101782, 101783, 101784, 101785, 101786 were selected from the ORFs having the nucleotide sequence represented by SEQ ID NO: 77042.

The probes having the nucleotide sequence represented by SEQ ID NOS: 101787, 101788, 101789, 101790, 101791, 101792, 101793, 101794, 101795, 101796, 101797 were selected from the ORFs having the nucleotide sequence represented by SEQ ID NO: 77043.

The probes having the nucleotide sequence represented by SEQ ID NOS: 101798, 101799, 101800, 101801, 101802, 101803, 101804, 101805, 101806, 101807, 101808 were selected from the ORFs having the nucleotide sequence represented by SEQ ID NO: 77044.

The probes having the nucleotide sequence represented by SEQ ID NOS: 101809, 101810, 101811, 101812, 101813, 101814, 101815, 101816, 101817, 101818, 101819 were selected from the ORFs having the nucleotide sequence represented by SEQ ID NO: 77045.

The probes having the nucleotide sequence represented by SEQ ID NOS: 101820, 101821, 101822, 101823, 101824, 101825, 101826, 101827, 101828, 101829, 101830 were selected from the ORFs having the nucleotide sequence represented by SEQ ID NO: 77046.

The probes having the nucleotide sequence represented by SEQ ID NOS: 101831, 101832, 101833, 101834, 101835, 101836, 101837, 101838, 101839, 101840, 101841 were selected from the ORFs having the nucleotide sequence represented by SEQ ID NO: 77047.

The probes having the nucleotide sequence represented by SEQ ID NOS: 101842, 101843, 101844, 101845, 101846, 101847, 101848, 101849, 101850, 101851, 101852 were selected from the ORFs having the nucleotide sequence represented by SEQ ID NO: 77048.

The probes having the nucleotide sequence represented by SEQ ID NOS: 101853, 101854, 101855, 101856, 101857, 101858, 101859, 101860, 101861, 101862, 101863 were selected from the ORFs having the nucleotide sequence represented by SEQ ID NO: 77049.

The probes having the nucleotide sequence represented by SEQ ID NOS: 101864, 101865, 101866, 101867, 101868, 101869, 101870, 101871, 101872, 101873, 101874 were selected from the ORFs having the nucleotide sequence represented by SEQ ID NO: 77050.

The probes having the nucleotide sequence represented by SEQ ID NOS: 101875, 101876, 101877, 101878, 101879, 101880, 101881, 101882, 101883, 101884, 101885 were selected from the ORFs having the nucleotide sequence represented by SEQ ID NO: 77051.

The probes having the nucleotide sequence represented by SEQ ID NOS: 101886, 101887, 101888, 101889, 101890, 101891, 101892, 101893, 101894, 101895, 101896 were selected from the ORFs having the nucleotide sequence represented by SEQ ID NO: 77052.

The probes having the nucleotide sequence represented by SEQ ID NOS: 101897, 101898, 101899, 101900, 101901, 101902, 101903, 101904, 101905, 101906, 101907 were selected from the ORFs having the nucleotide sequence represented by SEQ ID NO: 77053.

The probes having the nucleotide sequence represented by SEQ ID NOS: 101908, 101909, 101910, 101911, 101912, 101913, 101914, 101915, 101916, 101917, 101918 were selected from the ORFs having the nucleotide sequence represented by SEQ ID NO: 77054.

The probes having the nucleotide sequence represented by SEQ ID NOS: 101919, 101920, 101921, 101922, 101923, 101924, 101925, 101926, 101927, 101928, 101929 were selected from the ORFs having the nucleotide sequence represented by SEQ ID NO: 77055.

The probes having the nucleotide sequence represented by SEQ ID NOS: 101930, 101931, 101932, 101933, 101934, 101935, 101936, 101937, 101938, 101939, 101940 were selected from the ORFs having the nucleotide sequence represented by SEQ ID NO: 77056.

The probes having the nucleotide sequence represented by SEQ ID NOS: 101941, 101942, 101943, 101944, 101945, 101946, 101947, 101948, 101949, 101950, 101951 were selected from the ORFs having the nucleotide sequence represented by SEQ ID NO: 77057.

The probes having the nucleotide sequence represented by SEQ ID NOS: 101952, 101953, 101954, 101955, 101956, 101957, 101958, 101959, 101960, 101961, 101962 were selected from the ORFs having the nucleotide sequence represented by SEQ ID NO: 77058.

The probes having the nucleotide sequence represented by SEQ ID NOS: 101963, 101964, 101965, 101966, 101967, 101968, 101969, 101970, 101971, 101972, 101973 were selected from the ORFs having the nucleotide sequence represented by SEQ ID NO: 77059.

The probes having the nucleotide sequence represented by SEQ ID NOS: 101974, 101975, 101976, 101977, 101978, 101979, 101980, 101981, 101982, 101983, 101984 were selected from the ORFs having the nucleotide sequence represented by SEQ ID NO: 77060.

The probes having the nucleotide sequence represented by SEQ ID NOS: 101985, 101986, 101987, 101988, 101989, 101990, 101991, 101992, 101993, 101994, 101995 were selected from the ORFs having the nucleotide sequence represented by SEQ ID NO: 77061.

The probes having the nucleotide sequence represented by SEQ ID NOS: 101996, 101997, 101998, 101999, 102000, 102001, 102002, 102003, 102004, 102005, 102006 were selected from the ORFs having the nucleotide sequence represented by SEQ ID NO: 77062.

The probes having the nucleotide sequence represented by SEQ ID NOS: 102007, 102008, 102009, 102010, 102011, 102012, 102013, 102014, 102015, 102016, 102017 were selected from the ORFs having the nucleotide sequence represented by SEQ ID NO: 77063.

The probes having the nucleotide sequence represented by SEQ ID NOS: 102018, 102019, 102020, 102021, 102022, 102023, 102024, 102025, 102026, 102027, 102028 were selected from the ORFs having the nucleotide sequence represented by SEQ ID NO: 77064.

The probes having the nucleotide sequence represented by SEQ ID NOS: 102029, 102030, 102031, 102032, 102033, 102034, 102035, 102036, 102037, 102038, 102039 were selected from the ORFs having the nucleotide sequence represented by SEQ ID NO: 77065.

The probes having the nucleotide sequence represented by SEQ ID NOS: 102040, 102041, 102042, 102043, 102044, 102045, 102046, 102047, 102048, 102049, 102050 were selected from the ORFs having the nucleotide sequence represented by SEQ ID NO: 77066.

The probes having the nucleotide sequence represented by SEQ ID NOS: 102051, 102052, 102053, 102054, 102055, 102056, 102057, 102058, 102059, 102060, 102061 were selected from the ORFs having the nucleotide sequence represented by SEQ ID NO: 77067.

The probes having the nucleotide sequence represented by SEQ ID NOS: 102062, 102063, 102064, 102065, 102066, 102067, 102068, 102069, 102070, 102071, 102072 were selected from the ORFs having the nucleotide sequence represented by SEQ ID NO: 77068.

The probes having the nucleotide sequence represented by SEQ ID NOS: 102073, 102074, 102075, 102076, 102077, 102078, 102079, 102080, 102081, 102082, 102083 were selected from the ORFs having the nucleotide sequence represented by SEQ ID NO: 77069.

The probes having the nucleotide sequence represented by SEQ ID NOS: 102084, 102085, 102086, 102087, 102088, 102089, 102090, 102091, 102092, 102093, 102094 were selected from the ORFs having the nucleotide sequence represented by SEQ ID NO: 77070.

The probes having the nucleotide sequence represented by SEQ ID NOS: 102095, 102096, 102097, 102098, 102099, 102100, 102101, 102102, 102103, 102104, 102105 were selected from the ORFs having the nucleotide sequence represented by SEQ ID NO: 77071.

The probes having the nucleotide sequence represented by SEQ ID NOS: 102106, 102107, 102108, 102109, 102110, 102111, 102112, 102113, 102114, 102115, 102116 were selected from the ORFs having the nucleotide sequence represented by SEQ ID NO: 77072.

The probes having the nucleotide sequence represented by SEQ ID NOS: 102117, 102118, 102119, 102120, 102121, 102122, 102123, 102124, 102125, 102126, 102127 were selected from the ORFs having the nucleotide sequence represented by SEQ ID NO: 77073.

The probes having the nucleotide sequence represented by SEQ ID NOS: 102128, 102129, 102130, 102131, 102132, 102133, 102134, 102135, 102136, 102137, 102138 were selected from the ORFs having the nucleotide sequence represented by SEQ ID NO: 77074.

The probes having the nucleotide sequence represented by SEQ ID NOS: 102139, 102140, 102141, 102142, 102143, 102144, 102145, 102146, 102147, 102148, 102149 were selected from the ORFs having the nucleotide sequence represented by SEQ ID NO: 77075.

The probes having the nucleotide sequence represented by SEQ ID NOS: 102150, 102151, 102152, 102153, 102154, 102155, 102156, 102157, 102158, 102159, 102160 were selected from the ORFs having the nucleotide sequence represented by SEQ ID NO: 77076.

The probes having the nucleotide sequence represented by SEQ ID NOS: 102161, 102162, 102163, 102164, 102165, 102166, 102167, 102168, 102169, 102170, 102171 were selected from the ORFs having the nucleotide sequence represented by SEQ ID NO: 77077.

The probes having the nucleotide sequence represented by SEQ ID NOS: 102172, 102173, 102174, 102175, 102176, 102177, 102178, 102179, 102180, 102181, 102182 were selected from the ORFs having the nucleotide sequence represented by SEQ ID NO: 77078.

The probes having the nucleotide sequence represented by SEQ ID NOS: 102183, 102184, 102185, 102186, 102187, 102188, 102189, 102190, 102191, 102192, 102193 were selected from the ORFs having the nucleotide sequence represented by SEQ ID NO: 77079.

The probes having the nucleotide sequence represented by SEQ ID NOS: 102194, 102195, 102196, 102197, 102198, 102199, 102200, 102201, 102202, 102203, 102204 were selected from the ORFs having the nucleotide sequence represented by SEQ ID NO: 77080.

The probes having the nucleotide sequence represented by SEQ ID NOS: 102205, 102206, 102207, 102208, 102209, 102210, 102211, 102212, 102213, 102214, 102215 were selected from the ORFs having the nucleotide sequence represented by SEQ ID NO: 77081.

The probes having the nucleotide sequence represented by SEQ ID NOS: 102216, 102217, 102218, 102219, 102220, 102221, 102222, 102223, 102224, 102225, 102226 were selected from the ORFs having the nucleotide sequence represented by SEQ ID NO: 77082.

The probes having the nucleotide sequence represented by SEQ ID NOS: 102227, 102228, 102229, 102230, 102231, 102232, 102233, 102234, 102235, 102236, 102237 were selected from the ORFs having the nucleotide sequence represented by SEQ ID NO: 77083.

The probes having the nucleotide sequence represented by SEQ ID NOS: 102238, 102239, 102240, 102241, 102242, 102243, 102244, 102245, 102246, 102247, 102248 were selected from the ORFs having the nucleotide sequence represented by SEQ ID NO: 77084.

The probes having the nucleotide sequence represented by SEQ ID NOS: 102249, 102250, 102251, 102252, 102253, 102254, 102255, 102256, 102257, 102258, 102259 were selected from the ORFs having the nucleotide sequence represented by SEQ ID NO: 77085.

The probes having the nucleotide sequence represented by SEQ ID NOS: 102260, 102261, 102262, 102263, 102264, 102265, 102266, 102267, 102268, 102269, 102270 were selected from the ORFs having the nucleotide sequence represented by SEQ ID NO: 77086.

The probes having the nucleotide sequence represented by SEQ ID NOS: 102271, 102272, 102273, 102274, 102275, 102276, 102277, 102278, 102279, 102280, 102281 were selected from the ORFs having the nucleotide sequence represented by SEQ ID NO: 77087.

The probes having the nucleotide sequence represented by SEQ ID NOS: 102282, 102283, 102284, 102285, 102286, 102287, 102288, 102289, 102290, 102291, 102292 were selected from the ORFs having the nucleotide sequence represented by SEQ ID NO: 77088.

The probes having the nucleotide sequence represented by SEQ ID NOS: 102293, 102294, 102295, 102296, 102297, 102298, 102299, 102300, 102301, 102302, 102303 were selected from the ORFs having the nucleotide sequence represented by SEQ ID NO: 77089.

The probes having the nucleotide sequence represented by SEQ ID NOS: 102304, 102305, 102306, 102307, 102308, 102309, 102310, 102311, 102312, 102313, 102314 were selected from the ORFs having the nucleotide sequence represented by SEQ ID NO: 77090.

The probes having the nucleotide sequence represented by SEQ ID NOS: 102315, 102316, 102317, 102318, 102319, 102320, 102321, 102322, 102323, 102324, 102325 were selected from the ORFs having the nucleotide sequence represented by SEQ ID NO: 77091.

The probes having the nucleotide sequence represented by SEQ ID NOS: 102326, 102327, 102328, 102329, 102330, 102331, 102332, 102333, 102334, 102335, 102336 were selected from the ORFs having the nucleotide sequence represented by SEQ ID NO: 77092.

The probes having the nucleotide sequence represented by SEQ ID NOS: 102337, 102338, 102339, 102340, 102341, 102342, 102343, 102344, 102345, 102346, 102347 were selected from the ORFs having the nucleotide sequence represented by SEQ ID NO: 77093.

The probes having the nucleotide sequence represented by SEQ ID NOS: 102348, 102349, 102350, 102351, 102352, 102353, 102354, 102355, 102356, 102357, 102358 were selected from the ORFs having the nucleotide sequence represented by SEQ ID NO: 77094.

The probes having the nucleotide sequence represented by SEQ ID NOS: 102359, 102360, 102361, 102362, 102363, 102364, 102365, 102366, 102367, 102368, 102369 were selected from the ORFs having the nucleotide sequence represented by SEQ ID NO: 77095.

The probes having the nucleotide sequence represented by SEQ ID NOS: 102370, 102371, 102372, 102373, 102374, 102375, 102376, 102377, 102378, 102379, 102380 were selected from the ORFs having the nucleotide sequence represented by SEQ ID NO: 77096.

The probes having the nucleotide sequence represented by SEQ ID NOS: 102381, 102382, 102383, 102384, 102385, 102386, 102387, 102388, 102389, 102390, 102391 were selected from the ORFs having the nucleotide sequence represented by SEQ ID NO: 77097.

The probes having the nucleotide sequence represented by SEQ ID NOS: 102392, 102393, 102394, 102395, 102396, 102397, 102398, 102399, 102400, 102401, 102402 were selected from the ORFs having the nucleotide sequence represented by SEQ ID NO: 77098.

The probes having the nucleotide sequence represented by SEQ ID NOS: 102403, 102404, 102405, 102406, 102407, 102408, 102409, 102410, 102411, 102412, 102413 were selected from the ORFs having the nucleotide sequence represented by SEQ ID NO: 77099.

The probes having the nucleotide sequence represented by SEQ ID NOS: 102414, 102415, 102416, 102417, 102418, 102419, 102420, 102421, 102422, 102423, 102424 were selected from the ORFs having the nucleotide sequence represented by SEQ ID NO: 77100.

The probes having the nucleotide sequence represented by SEQ ID NOS: 102425, 102426, 102427, 102428, 102429, 102430, 102431, 102432, 102433, 102434, 102435 were selected from the ORFs having the nucleotide sequence represented by SEQ ID NO: 77101.

The probes having the nucleotide sequence represented by SEQ ID NOS: 102436, 102437, 102438, 102439, 102440, 102441, 102442, 102443, 102444, 102445, 102446 were selected from the ORFs having the nucleotide sequence represented by SEQ ID NO: 77102.

The probes having the nucleotide sequence represented by SEQ ID NOS: 102447, 102448, 102449, 102450, 102451, 102452, 102453, 102454, 102455, 102456, 102457 were selected from the ORFs having the nucleotide sequence represented by SEQ ID NO: 77103.

The probes having the nucleotide sequence represented by SEQ ID NOS: 102458, 102459, 102460, 102461, 102462, 102463, 102464, 102465, 102466, 102467, 102468 were selected from the ORFs having the nucleotide sequence represented by SEQ ID NO: 77104.

The probes having the nucleotide sequence represented by SEQ ID NOS: 102469, 102470, 102471, 102472, 102473, 102474, 102475, 102476, 102477, 102478, 102479 were selected from the ORFs having the nucleotide sequence represented by SEQ ID NO: 77105.

The probes having the nucleotide sequence represented by SEQ ID NOS: 102480, 102481, 102482, 102483, 102484, 102485, 102486, 102487, 102488, 102489, 102490 were selected from the ORFs having the nucleotide sequence represented by SEQ ID NO: 77106.

The probes having the nucleotide sequence represented by SEQ ID NOS: 102491, 102492, 102493, 102494, 102495, 102496, 102497, 102498, 102499, 102500, 102501 were selected from the ORFs having the nucleotide sequence represented by SEQ ID NO: 77107.

The probes having the nucleotide sequence represented by SEQ ID NOS: 102502, 102503, 102504, 102505, 102506, 102507, 102508, 102509, 102510, 102511, 102512 were selected from the ORFs having the nucleotide sequence represented by SEQ ID NO: 77108.

The probes having the nucleotide sequence represented by SEQ ID NOS: 102513, 102514, 102515, 102516, 102517, 102518, 102519, 102520, 102521, 102522, 102523 were selected from the ORFs having the nucleotide sequence represented by SEQ ID NO: 77109.

The probes having the nucleotide sequence represented by SEQ ID NOS: 102524, 102525, 102526, 102527, 102528, 102529, 102530, 102531, 102532, 102533, 102534 were selected from the ORFs having the nucleotide sequence represented by SEQ ID NO: 77110.

The probes having the nucleotide sequence represented by SEQ ID NOS: 102535, 102536, 102537, 102538, 102539, 102540, 102541, 102542, 102543, 102544, 102545 were selected from the ORFs having the nucleotide sequence represented by SEQ ID NO: 77111.

The probes having the nucleotide sequence represented by SEQ ID NOS: 102546, 102547, 102548, 102549, 102550, 102551, 102552, 102553, 102554, 102555, 102556 were selected from the ORFs having the nucleotide sequence represented by SEQ ID NO: 77112.

The probes having the nucleotide sequence represented by SEQ ID NOS: 102557, 102558, 102559, 102560, 102561, 102562, 102563, 102564, 102565, 102566, 102567 were selected from the ORFs having the nucleotide sequence represented by SEQ ID NO: 77113.

The probes having the nucleotide sequence represented by SEQ ID NOS: 102568, 102569, 102570, 102571, 102572, 102573, 102574, 102575, 102576, 102577, 102578 were selected from the ORFs having the nucleotide sequence represented by SEQ ID NO: 77114.

The probes having the nucleotide sequence represented by SEQ ID NOS: 102579, 102580, 102581, 102582, 102583, 102584, 102585, 102586, 102587, 102588, 102589 were selected from the ORFs having the nucleotide sequence represented by SEQ ID NO: 77115.

The probes having the nucleotide sequence represented by SEQ ID NOS: 102590, 102591, 102592, 102593, 102594, 102595, 102596, 102597, 102598, 102599, 102600 were selected from the ORFs having the nucleotide sequence represented by SEQ ID NO: 77116.

The probes having the nucleotide sequence represented by SEQ ID NOS: 102601, 102602, 102603, 102604, 102605, 102606, 102607, 102608, 102609, 102610, 102611 were selected from the ORFs having the nucleotide sequence represented by SEQ ID NO: 77117.

The probes having the nucleotide sequence represented by SEQ ID NOS: 102612, 102613, 102614, 102615, 102616, 102617, 102618, 102619, 102620, 102621, 102622 were selected from the ORFs having the nucleotide sequence represented by SEQ ID NO: 77118.

The probes having the nucleotide sequence represented by SEQ ID NOS: 102623, 102624, 102625, 102626, 102627, 102628, 102629, 102630, 102631, 102632, 102633 were selected from the ORFs having the nucleotide sequence represented by SEQ ID NO: 77119.

The probes having the nucleotide sequence represented by SEQ ID NOS: 102634, 102635, 102636, 102637, 102638, 102639, 102640, 102641, 102642, 102643, 102644 were selected from the ORFs having the nucleotide sequence represented by SEQ ID NO: 77120.

The probes having the nucleotide sequence represented by SEQ ID NOS: 102645, 102646, 102647, 102648, 102649, 102650, 102651, 102652, 102653, 102654, 102655 were selected from the ORFs having the nucleotide sequence represented by SEQ ID NO: 77121.

The probes having the nucleotide sequence represented by SEQ ID NOS: 102656, 102657, 102658, 102659, 102660, 102661, 102662, 102663, 102664, 102665, 102666 were selected from the ORFs having the nucleotide sequence represented by SEQ ID NO: 77122.

The probes having the nucleotide sequence represented by SEQ ID NOS: 102667, 102668, 102669, 102670, 102671, 102672, 102673, 102674, 102675, 102676, 102677 were selected from the ORFs having the nucleotide sequence represented by SEQ ID NO: 77123.

The probes having the nucleotide sequence represented by SEQ ID NOS: 102678, 102679, 102680, 102681, 102682, 102683, 102684, 102685, 102686, 102687, 102688 were selected from the ORFs having the nucleotide sequence represented by SEQ ID NO: 77124.

The probes having the nucleotide sequence represented by SEQ ID NOS: 102689, 102690, 102691, 102692, 102693, 102694, 102695, 102696, 102697, 102698, 102699 were selected from the ORFs having the nucleotide sequence represented by SEQ ID NO: 77125.

The probes having the nucleotide sequence represented by SEQ ID NOS: 102700, 102701, 102702, 102703, 102704, 102705, 102706, 102707, 102708, 102709, 102710 were selected from the ORFs having the nucleotide sequence represented by SEQ ID NO: 77126.

The probes having the nucleotide sequence represented by SEQ ID NOS: 102711, 102712, 102713, 102714, 102715, 102716, 102717, 102718, 102719, 102720, 102721 were selected from the ORFs having the nucleotide sequence represented by SEQ ID NO: 77127.

The probes having the nucleotide sequence represented by SEQ ID NOS: 102722, 102723, 102724, 102725, 102726, 102727, 102728, 102729, 102730, 102731, 102732 were selected from the ORFs having the nucleotide sequence represented by SEQ ID NO: 77128.

The probes having the nucleotide sequence represented by SEQ ID NOS: 102733, 102734, 102735, 102736, 102737, 102738, 102739, 102740, 102741, 102742, 102743 were selected from the ORFs having the nucleotide sequence represented by SEQ ID NO: 77129.

The probes having the nucleotide sequence represented by SEQ ID NOS: 102744, 102745, 102746, 102747, 102748, 102749, 102750, 102751, 102752, 102753, 102754 were selected from the ORFs having the nucleotide sequence represented by SEQ ID NO: 77130.

The probes having the nucleotide sequence represented by SEQ ID NOS: 102755, 102756, 102757, 102758, 102759, 102760, 102761, 102762, 102763, 102764, 102765 were selected from the ORFs having the nucleotide sequence represented by SEQ ID NO: 77131.

The probes having the nucleotide sequence represented by SEQ ID NOS: 102766, 102767, 102768, 102769, 102770, 102771, 102772, 102773, 102774, 102775, 102776 were selected from the ORFs having the nucleotide sequence represented by SEQ ID NO: 77132.

The probes having the nucleotide sequence represented by SEQ ID NOS: 102777, 102778, 102779, 102780, 102781, 102782, 102783, 102784, 102785, 102786, 102787 were selected from the ORFs having the nucleotide sequence represented by SEQ ID NO: 77133.

The probes having the nucleotide sequence represented by SEQ ID NOS: 102788, 102789, 102790, 102791, 102792, 102793, 102794, 102795, 102796, 102797, 102798 were selected from the ORFs having the nucleotide sequence represented by SEQ ID NO: 77134.

The probes having the nucleotide sequence represented by SEQ ID NOS: 102799, 102800, 102801, 102802, 102803, 102804, 102805, 102806, 102807, 102808, 102809 were selected from the ORFs having the nucleotide sequence represented by SEQ ID NO: 77135.

The probes having the nucleotide sequence represented by SEQ ID NOS: 102810, 102811, 102812, 102813, 102814, 102815, 102816, 102817, 102818, 102819, 102820 were selected from the ORFs having the nucleotide sequence represented by SEQ ID NO: 77136.

The probes having the nucleotide sequence represented by SEQ ID NOS: 102821, 102822, 102823, 102824, 102825, 102826, 102827, 102828, 102829, 102830, 102831 were selected from the ORFs having the nucleotide sequence represented by SEQ ID NO: 77137.

The probes having the nucleotide sequence represented by SEQ ID NOS: 102832, 102833, 102834, 102835, 102836, 102837, 102838, 102839, 102840, 102841, 102842 were selected from the ORFs having the nucleotide sequence represented by SEQ ID NO: 77138.

The probes having the nucleotide sequence represented by SEQ ID NOS: 102843, 102844, 102845, 102846, 102847, 102848, 102849, 102850, 102851, 102852, 102853 were selected from the ORFs having the nucleotide sequence represented by SEQ ID NO: 77139.

The probes having the nucleotide sequence represented by SEQ ID NOS: 102854, 102855, 102856, 102857, 102858, 102859, 102860, 102861, 102862, 102863, 102864 were selected from the ORFs having the nucleotide sequence represented by SEQ ID NO: 77140.

The probes having the nucleotide sequence represented by SEQ ID NOS: 102865, 102866, 102867, 102868, 102869, 102870, 102871, 102872, 102873, 102874, 102875 were selected from the ORFs having the nucleotide sequence represented by SEQ ID NO: 77141.

The probes having the nucleotide sequence represented by SEQ ID NOS: 102876, 102877, 102878, 102879, 102880, 102881, 102882, 102883, 102884, 102885, 102886 were selected from the ORFs having the nucleotide sequence represented by SEQ ID NO: 77142.

The probes having the nucleotide sequence represented by SEQ ID NOS: 102887, 102888, 102889, 102890, 102891, 102892, 102893, 102894, 102895, 102896, 102897 were selected from the ORFs having the nucleotide sequence represented by SEQ ID NO: 77143.

The probes having the nucleotide sequence represented by SEQ ID NOS: 102898, 102899, 102900, 102901, 102902, 102903, 102904, 102905, 102906, 102907, 102908 were selected from the ORFs having the nucleotide sequence represented by SEQ ID NO: 77144.

The probes having the nucleotide sequence represented by SEQ ID NOS: 102909, 102910, 102911, 102912, 102913, 102914, 102915, 102916, 102917, 102918, 102919 were selected from the ORFs having the nucleotide sequence represented by SEQ ID NO: 77145.

The probes having the nucleotide sequence represented by SEQ ID NOS: 102920, 102921, 102922, 102923, 102924, 102925, 102926, 102927, 102928, 102929, 102930 were selected from the ORFs having the nucleotide sequence represented by SEQ ID NO: 77146.

The probes having the nucleotide sequence represented by SEQ ID NOS: 102931, 102932, 102933, 102934, 102935, 102936, 102937, 102938, 102939, 102940, 102941 were selected from the ORFs having the nucleotide sequence represented by SEQ ID NO: 77147.

The probes having the nucleotide sequence represented by SEQ ID NOS: 102942, 102943, 102944, 102945, 102946, 102947, 102948, 102949, 102950, 102951, 102952 were selected from the ORFs having the nucleotide sequence represented by SEQ ID NO: 77148.

The probes having the nucleotide sequence represented by SEQ ID NOS: 102953, 102954, 102955, 102956, 102957, 102958, 102959, 102960, 102961, 102962, 102963 were selected from the ORFs having the nucleotide sequence represented by SEQ ID NO: 77149.

The probes having the nucleotide sequence represented by SEQ ID NOS: 102964, 102965, 102966, 102967, 102968, 102969, 102970, 102971, 102972, 102973, 102974 were selected from the ORFs having the nucleotide sequence represented by SEQ ID NO: 77150.

The probes having the nucleotide sequence represented by SEQ ID NOS: 102975, 102976, 102977, 102978, 102979, 102980, 102981, 102982, 102983, 102984, 102985 were selected from the ORFs having the nucleotide sequence represented by SEQ ID NO: 77151.

The probes having the nucleotide sequence represented by SEQ ID NOS: 102986, 102987, 102988, 102989, 102990, 102991, 102992, 102993, 102994, 102995, 102996 were selected from the ORFs having the nucleotide sequence represented by SEQ ID NO: 77152.

The probes having the nucleotide sequence represented by SEQ ID NOS: 102997, 102998, 102999, 103000, 103001, 103002, 103003, 103004, 103005, 103006, 103007 were selected from the ORFs having the nucleotide sequence represented by SEQ ID NO: 77153.

The probes having the nucleotide sequence represented by SEQ ID NOS: 103008, 103009, 103010, 103011, 103012, 103013, 103014, 103015, 103016, 103017, 103018 were selected from the ORFs having the nucleotide sequence represented by SEQ ID NO: 77154.

The probes having the nucleotide sequence represented by SEQ ID NOS: 103019, 103020, 103021, 103022, 103023, 103024, 103025, 103026, 103027, 103028, 103029 were selected from the ORFs having the nucleotide sequence represented by SEQ ID NO: 77155.

The probes having the nucleotide sequence represented by SEQ ID NOS: 103030, 103031, 103032, 103033, 103034, 103035, 103036, 103037, 103038, 103039, 103040 were selected from the ORFs having the nucleotide sequence represented by SEQ ID NO: 77156.

The probes having the nucleotide sequence represented by SEQ ID NOS: 103041, 103042, 103043, 103044, 103045, 103046, 103047, 103048, 103049, 103050, 103051 were selected from the ORFs having the nucleotide sequence represented by SEQ ID NO: 77157.

The probes having the nucleotide sequence represented by SEQ ID NOS: 103052, 103053, 103054, 103055, 103056, 103057, 103058, 103059, 103060, 103061, 103062 were selected from the ORFs having the nucleotide sequence represented by SEQ ID NO: 77158.

The probes having the nucleotide sequence represented by SEQ ID NOS: 103063, 103064, 103065, 103066, 103067, 103068, 103069, 103070, 103071, 103072, 103073 were selected from the ORFs having the nucleotide sequence represented by SEQ ID NO: 77159.

The probes having the nucleotide sequence represented by SEQ ID NOS: 103074, 103075, 103076, 103077, 103078, 103079, 103080, 103081, 103082, 103083, 103084 were selected from the ORFs having the nucleotide sequence represented by SEQ ID NO: 77160.

The probes having the nucleotide sequence represented by SEQ ID NOS: 103085, 103086, 103087, 103088, 103089, 103090, 103091, 103092, 103093, 103094, 103095 were selected from the ORFs having the nucleotide sequence represented by SEQ ID NO: 77161.

The probes having the nucleotide sequence represented by SEQ ID NOS: 103096, 103097, 103098, 103099, 103100, 103101, 103102, 103103, 103104, 103105, 103106 were selected from the ORFs having the nucleotide sequence represented by SEQ ID NO: 77162.

The probes having the nucleotide sequence represented by SEQ ID NOS: 103107, 103108, 103109, 103110, 103111, 103112, 103113, 103114, 103115, 103116, 103117 were selected from the ORFs having the nucleotide sequence represented by SEQ ID NO: 77163.

The probes having the nucleotide sequence represented by SEQ ID NOS: 103118, 103119, 103120, 103121, 103122, 103123, 103124, 103125, 103126, 103127, 103128 were selected from the ORFs having the nucleotide sequence represented by SEQ ID NO: 77164.

The probes having the nucleotide sequence represented by SEQ ID NOS: 103129, 103130, 103131, 103132, 103133, 103134, 103135, 103136, 103137, 103138, 103139 were selected from the ORFs having the nucleotide sequence represented by SEQ ID NO: 77165.

The probes having the nucleotide sequence represented by SEQ ID NOS: 103140, 103141, 103142, 103143, 103144, 103145, 103146, 103147, 103148, 103149, 103150 were selected from the ORFs having the nucleotide sequence represented by SEQ ID NO: 77166.

The probes having the nucleotide sequence represented by SEQ ID NOS: 103151, 103152, 103153, 103154, 103155, 103156, 103157, 103158, 103159, 103160, 103161, 103162, 103163, 103164, 103165, 103166, 103167, 103168, 103169, 103170, 103171, 103172 were selected from the ORFs having the nucleotide sequence represented by SEQ ID NO: 77167.

The probes having the nucleotide sequence represented by SEQ ID NOS: 103173, 103174, 103175, 103176, 103177, 103178, 103179, 103180, 103181, 103182, 103183 were selected from the ORFs having the nucleotide sequence represented by SEQ ID NO: 77168.

The probes having the nucleotide sequence represented by SEQ ID NOS: 103184, 103185, 103186, 103187, 103188, 103189, 103190, 103191, 103192, 103193, 103194 were selected from the ORFs having the nucleotide sequence represented by SEQ ID NO: 77169.

The probes having the nucleotide sequence represented by SEQ ID NOS: 103195, 103196, 103197, 103198, 103199, 103200, 103201, 103202, 103203, 103204, 103205 were selected from the ORFs having the nucleotide sequence represented by SEQ ID NO: 77170.

The probes having the nucleotide sequence represented by SEQ ID NOS: 103206, 103207, 103208, 103209, 103210, 103211, 103212, 103213, 103214, 103215, 103216 were selected from the ORFs having the nucleotide sequence represented by SEQ ID NO: 77171.

The probes having the nucleotide sequence represented by SEQ ID NOS: 103217, 103218, 103219, 103220, 103221, 103222, 103223, 103224, 103225, 103226, 103227 were selected from the ORFs having the nucleotide sequence represented by SEQ ID NO: 77172.

The probes having the nucleotide sequence represented by SEQ ID NOS: 103228, 103229, 103230, 103231, 103232, 103233, 103234, 103235, 103236, 103237, 103238 were selected from the ORFs having the nucleotide sequence represented by SEQ ID NO: 77173.

The probes having the nucleotide sequence represented by SEQ ID NOS: 103239, 103240, 103241, 103242, 103243, 103244, 103245, 103246, 103247, 103248, 103249 were selected from the ORFs having the nucleotide sequence represented by SEQ ID NO: 77174.

The probes having the nucleotide sequence represented by SEQ ID NOS: 103250, 103251, 103252, 103253, 103254, 103255, 103256, 103257, 103258, 103259, 103260 were selected from the ORFs having the nucleotide sequence represented by SEQ ID NO: 77175.

The probes having the nucleotide sequence represented by SEQ ID NOS: 103261, 103262, 103263, 103264, 103265, 103266, 103267, 103268, 103269, 103270, 103271 were selected from the ORFs having the nucleotide sequence represented by SEQ ID NO: 77176.

The probes having the nucleotide sequence represented by SEQ ID NOS: 103272, 103273, 103274, 103275, 103276, 103277, 103278, 103279, 103280, 103281, 103282 were selected from the ORFs having the nucleotide sequence represented by SEQ ID NO: 77177.

The probes having the nucleotide sequence represented by SEQ ID NOS: 103283, 103284, 103285, 103286, 103287, 103288, 103289, 103290, 103291, 103292, 103293 were selected from the ORFs having the nucleotide sequence represented by SEQ ID NO: 77178.

The probes having the nucleotide sequence represented by SEQ ID NOS: 103294, 103295, 103296, 103297, 103298, 103299, 103300, 103301, 103302, 103303, 103304 were selected from the ORFs having the nucleotide sequence represented by SEQ ID NO: 77179.

The probes having the nucleotide sequence represented by SEQ ID NOS: 103305, 103306, 103307, 103308, 103309, 103310, 103311, 103312, 103313, 103314, 103315 were selected from the ORFs having the nucleotide sequence represented by SEQ ID NO: 77180.

The probes having the nucleotide sequence represented by SEQ ID NOS: 103316, 103317, 103318, 103319, 103320, 103321, 103322, 103323, 103324, 103325, 103326 were selected from the ORFs having the nucleotide sequence represented by SEQ ID NO: 77181.

The probes having the nucleotide sequence represented by SEQ ID NOS: 103327, 103328, 103329, 103330, 103331, 103332, 103333, 103334, 103335, 103336, 103337 were selected from the ORFs having the nucleotide sequence represented by SEQ ID NO: 77182.

The probes having the nucleotide sequence represented by SEQ ID NOS: 103338, 103339, 103340, 103341, 103342, 103343, 103344, 103345, 103346, 103347, 103348 were selected from the ORFs having the nucleotide sequence represented by SEQ ID NO: 77183.

The probes having the nucleotide sequence represented by SEQ ID NOS: 103349, 103350, 103351, 103352, 103353, 103354, 103355, 103356, 103357, 103358, 103359 were selected from the ORFs having the nucleotide sequence represented by SEQ ID NO: 77184.

The probes having the nucleotide sequence represented by SEQ ID NOS: 103360, 103361, 103362, 103363, 103364, 103365, 103366, 103367, 103368, 103369, 103370 were selected from the ORFs having the nucleotide sequence represented by SEQ ID NO: 77185.

The probes having the nucleotide sequence represented by SEQ ID NOS: 103371, 103372, 103373, 103374, 103375, 103376, 103377, 103378, 103379, 103380, 103381 were selected from the ORFs having the nucleotide sequence represented by SEQ ID NO: 77186.

The probes having the nucleotide sequence represented by SEQ ID NOS: 103382, 103383, 103384, 103385, 103386, 103387, 103388, 103389, 103390, 103391, 103392 were selected from the ORFs having the nucleotide sequence represented by SEQ ID NO: 77187.

The probes having the nucleotide sequence represented by SEQ ID NOS: 103393, 103394, 103395, 103396, 103397, 103398, 103399, 103400, 103401, 103402, 103403 were selected from the ORFs having the nucleotide sequence represented by SEQ ID NO: 77188.

The probes having the nucleotide sequence represented by SEQ ID NOS: 103404, 103405, 103406, 103407, 103408, 103409, 103410, 103411, 103412, 103413, 103414 were selected from the ORFs having the nucleotide sequence represented by SEQ ID NO: 77189.

The probes having the nucleotide sequence represented by SEQ ID NOS: 103415, 103416, 103417, 103418, 103419, 103420, 103421, 103422, 103423, 103424, 103425 were selected from the ORFs having the nucleotide sequence represented by SEQ ID NO: 77190.

The probes having the nucleotide sequence represented by SEQ ID NOS: 103426, 103427, 103428, 103429, 103430, 103431, 103432, 103433, 103434, 103435, 103436 were selected from the ORFs having the nucleotide sequence represented by SEQ ID NO: 77191.

The probes having the nucleotide sequence represented by SEQ ID NOS: 103437, 103438, 103439, 103440, 103441, 103442, 103443, 103444, 103445, 103446, 103447 were selected from the ORFs having the nucleotide sequence represented by SEQ ID NO: 77192.

The probes having the nucleotide sequence represented by SEQ ID NOS: 103448, 103449, 103450, 103451, 103452, 103453, 103454, 103455, 103456, 103457, 103458 were selected from the ORFs having the nucleotide sequence represented by SEQ ID NO: 77193.

The probes having the nucleotide sequence represented by SEQ ID NOS: 103459, 103460, 103461, 103462, 103463, 103464, 103465, 103466, 103467, 103468, 103469 were selected from the ORFs having the nucleotide sequence represented by SEQ ID NO: 77194.

The probes having the nucleotide sequence represented by SEQ ID NOS: 103470, 103471, 103472, 103473, 103474, 103475, 103476, 103477, 103478, 103479, 103480 were selected from the ORFs having the nucleotide sequence represented by SEQ ID NO: 77195.

The probes having the nucleotide sequence represented by SEQ ID NOS: 103481, 103482, 103483, 103484, 103485, 103486, 103487, 103488, 103489, 103490, 103491 were selected from the ORFs having the nucleotide sequence represented by SEQ ID NO: 77196.

The probes having the nucleotide sequence represented by SEQ ID NOS: 103492, 103493, 103494, 103495, 103496, 103497, 103498, 103499, 103500, 103501, 103502, 103503, 103504, 103505, 103506, 103507, 103508, 103509, 103510, 103511, 103512, 103513 were selected from the ORFs having the nucleotide sequence represented by SEQ ID NO: 77197.

The probes having the nucleotide sequence represented by SEQ ID NOS: 103514, 103515, 103516, 103517, 103518, 103519, 103520, 103521, 103522, 103523, 103524 were selected from the ORFs having the nucleotide sequence represented by SEQ ID NO: 77198.

The probes having the nucleotide sequence represented by SEQ ID NOS: 103525, 103526, 103527, 103528, 103529, 103530, 103531, 103532, 103533, 103534, 103535 were selected from the ORFs having the nucleotide sequence represented by SEQ ID NO: 77199.

The probes having the nucleotide sequence represented by SEQ ID NOS: 103536, 103537, 103538, 103539, 103540, 103541, 103542, 103543, 103544, 103545, 103546 were selected from the ORFs having the nucleotide sequence represented by SEQ ID NO: 77200.

The probes having the nucleotide sequence represented by SEQ ID NOS: 103547, 103548, 103549, 103550, 103551, 103552, 103553, 103554, 103555, 103556, 103557 were selected from the ORFs having the nucleotide sequence represented by SEQ ID NO: 77201.

The probes having the nucleotide sequence represented by SEQ ID NOS: 103558, 103559, 103560, 103561, 103562, 103563, 103564, 103565, 103566, 103567, 103568 were selected from the ORFs having the nucleotide sequence represented by SEQ ID NO: 77202.

The probes having the nucleotide sequence represented by SEQ ID NOS: 103569, 103570, 103571, 103572, 103573, 103574, 103575, 103576, 103577, 103578, 103579 were selected from the ORFs having the nucleotide sequence represented by SEQ ID NO: 77203.

The probes having the nucleotide sequence represented by SEQ ID NOS: 103580, 103581, 103582, 103583, 103584, 103585, 103586, 103587, 103588, 103589, 103590 were selected from the ORFs having the nucleotide sequence represented by SEQ ID NO: 77204.

The probes having the nucleotide sequence represented by SEQ ID NOS: 103591, 103592, 103593, 103594, 103595, 103596, 103597, 103598, 103599, 103600, 103601 were selected from the ORFs having the nucleotide sequence represented by SEQ ID NO: 77205.

The probes having the nucleotide sequence represented by SEQ ID NOS: 103602, 103603, 103604, 103605, 103606, 103607, 103608, 103609, 103610, 103611, 103612 were selected from the ORFs having the nucleotide sequence represented by SEQ ID NO: 77206.

The probes having the nucleotide sequence represented by SEQ ID NOS: 103613, 103614, 103615, 103616, 103617, 103618, 103619, 103620, 103621, 103622, 103623 were selected from the ORFs having the nucleotide sequence represented by SEQ ID NO: 77207.

The probes having the nucleotide sequence represented by SEQ ID NOS: 103624, 103625, 103626, 103627, 103628, 103629, 103630, 103631, 103632, 103633, 103634 were selected from the ORFs having the nucleotide sequence represented by SEQ ID NO: 77208.

The probes having the nucleotide sequence represented by SEQ ID NOS: 103635, 103636, 103637, 103638, 103639, 103640, 103641, 103642, 103643, 103644, 103645 were selected from the ORFs having the nucleotide sequence represented by SEQ ID NO: 77209.

The probes having the nucleotide sequence represented by SEQ ID NOS: 103646, 103647, 103648, 103649, 103650, 103651, 103652, 103653, 103654, 103655, 103656 were selected from the ORFs having the nucleotide sequence represented by SEQ ID NO: 77210.

The probes having the nucleotide sequence represented by SEQ ID NOS: 103657, 103658, 103659, 103660, 103661, 103662, 103663, 103664, 103665, 103666, 103667 were selected from the ORFs having the nucleotide sequence represented by SEQ ID NO: 77211.

The probes having the nucleotide sequence represented by SEQ ID NOS: 103668, 103669, 103670, 103671, 103672, 103673, 103674, 103675, 103676, 103677, 103678 were selected from the ORFs having the nucleotide sequence represented by SEQ ID NO: 77212.

The probes having the nucleotide sequence represented by SEQ ID NOS: 103679, 103680, 103681, 103682, 103683, 103684, 103685, 103686, 103687, 103688, 103689 were selected from the ORFs having the nucleotide sequence represented by SEQ ID NO: 77213.

The probes having the nucleotide sequence represented by SEQ ID NOS: 103690, 103691, 103692, 103693, 103694, 103695, 103696, 103697, 103698, 103699, 103700 were selected from the ORFs having the nucleotide sequence represented by SEQ ID NO: 77214.

The probes having the nucleotide sequence represented by SEQ ID NOS: 103701, 103702, 103703, 103704, 103705, 103706, 103707, 103708, 103709, 103710, 103711 were selected from the ORFs having the nucleotide sequence represented by SEQ ID NO: 77215.

The probes having the nucleotide sequence represented by SEQ ID NOS: 103712, 103713, 103714, 103715, 103716, 103717, 103718, 103719, 103720, 103721, 103722 were selected from the ORFs having the nucleotide sequence represented by SEQ ID NO: 77216.

The probes having the nucleotide sequence represented by SEQ ID NOS: 103723, 103724, 103725, 103726, 103727, 103728, 103729, 103730, 103731, 103732, 103733 were selected from the ORFs having the nucleotide sequence represented by SEQ ID NO: 77217.

The probes having the nucleotide sequence represented by SEQ ID NOS: 103734, 103735, 103736, 103737, 103738, 103739, 103740, 103741, 103742, 103743, 103744 were selected from the ORFs having the nucleotide sequence represented by SEQ ID NO: 77218.

The probes having the nucleotide sequence represented by SEQ ID NOS: 103745, 103746, 103747, 103748, 103749, 103750, 103751, 103752, 103753, 103754, 103755 were selected from the ORFs having the nucleotide sequence represented by SEQ ID NO: 77219.

The probes having the nucleotide sequence represented by SEQ ID NOS: 103756, 103757, 103758, 103759, 103760, 103761, 103762, 103763, 103764, 103765, 103766 were selected from the ORFs having the nucleotide sequence represented by SEQ ID NO: 77220.

The probes having the nucleotide sequence represented by SEQ ID NOS: 103767, 103768, 103769, 103770, 103771, 103772, 103773, 103774, 103775, 103776, 103777 were selected from the ORFs having the nucleotide sequence represented by SEQ ID NO: 77221.

The probes having the nucleotide sequence represented by SEQ ID NOS: 103778, 103779, 103780, 103781, 103782, 103783, 103784, 103785, 103786, 103787, 103788 were selected from the ORFs having the nucleotide sequence represented by SEQ ID NO: 77222.

The probes having the nucleotide sequence represented by SEQ ID NOS: 103789, 103790, 103791, 103792, 103793, 103794, 103795, 103796, 103797, 103798, 103799 were selected from the ORFs having the nucleotide sequence represented by SEQ ID NO: 77223.

The probes having the nucleotide sequence represented by SEQ ID NOS: 103800, 103801, 103802, 103803, 103804, 103805, 103806, 103807, 103808, 103809, 103810 were selected from the ORFs having the nucleotide sequence represented by SEQ ID NO: 77224.

The probes having the nucleotide sequence represented by SEQ ID NOS: 103811, 103812, 103813, 103814, 103815, 103816, 103817, 103818, 103819, 103820, 103821 were selected from the ORFs having the nucleotide sequence represented by SEQ ID NO: 77225.

The probes having the nucleotide sequence represented by SEQ ID NOS: 103822, 103823, 103824, 103825, 103826, 103827, 103828, 103829, 103830, 103831, 103832 were selected from the ORFs having the nucleotide sequence represented by SEQ ID NO: 77226.

The probes having the nucleotide sequence represented by SEQ ID NOS: 103833, 103834, 103835, 103836, 103837, 103838, 103839, 103840, 103841, 103842, 103843 were selected from the ORFs having the nucleotide sequence represented by SEQ ID NO: 77227.

The probes having the nucleotide sequence represented by SEQ ID NOS: 103844, 103845, 1.03846, 103847, 103848, 103849, 103850, 103851, 103852, 103853, 103854 were selected from the ORFs having the nucleotide sequence represented by SEQ ID NO: 77228.

The probes having the nucleotide sequence represented by SEQ ID NOS: 103855, 103856, 103857, 103858, 103859, 103860, 103861, 103862, 103863, 103864, 103865 were selected from the ORFs having the nucleotide sequence represented by SEQ ID NO: 77229.

The probes having the nucleotide sequence represented by SEQ ID NOS: 103866, 103867, 103868, 103869, 103870, 103871, 103872, 103873, 103874, 103875, 103876 were selected from the ORFs having the nucleotide sequence represented by SEQ ID NO: 77230.

The probes having the nucleotide sequence represented by SEQ ID NOS: 103877, 103878, 103879, 103880, 103881, 103882, 103883, 103884, 103885, 103886, 103887 were selected from the ORFs having the nucleotide sequence represented by SEQ ID NO: 77231.

The probes having the nucleotide sequence represented by SEQ ID NOS: 103888, 103889, 103890, 103891, 103892, 103893, 103894, 103895, 103896, 103897, 103898 were selected from the ORFs having the nucleotide sequence represented by SEQ ID NO: 77232.

The probes having the nucleotide sequence represented by SEQ ID NOS: 103899, 103900, 103901, 103902, 103903, 103904, 103905, 103906, 103907, 103908, 103909 were selected from the ORFs having the nucleotide sequence represented by SEQ ID NO: 77233.

The probes having the nucleotide sequence represented by SEQ ID NOS: 103910, 103911, 103912, 103913, 103914, 103915, 103916, 103917, 103918, 103919, 103920 were selected from the ORFs having the nucleotide sequence represented by SEQ ID NO: 77234.

The probes having the nucleotide sequence represented by SEQ ID NOS: 103921, 103922, 103923, 103924, 103925, 103926, 103927, 103928, 103929, 103930, 103931 were selected from the ORFs having the nucleotide sequence represented by SEQ ID NO: 77235.

The probes having the nucleotide sequence represented by SEQ ID NOS: 103932, 103933, 103934, 103935, 103936, 103937, 103938, 103939, 103940, 103941, 103942 were selected from the ORFs having the nucleotide sequence represented by SEQ ID NO: 77236.

The probes having the nucleotide sequence represented by SEQ ID NOS: 103943, 103944, 103945, 103946, 103947, 103948, 103949, 103950, 103951, 103952, 103953 were selected from the ORFs having the nucleotide sequence represented by SEQ ID NO: 77237.

The probes having the nucleotide sequence represented by SEQ ID NOS: 103954, 103955, 103956, 103957, 103958, 103959, 103960, 103961, 103962, 103963, 103964 were selected from the ORFs having the nucleotide sequence represented by SEQ ID NO: 77238.

The probes having the nucleotide sequence represented by SEQ ID NOS: 103965, 103966, 103967, 103968, 103969, 103970, 103971, 103972, 103973, 103974, 103975 were selected from the ORFs having the nucleotide sequence represented by SEQ ID NO: 77239.

The probes having the nucleotide sequence represented by SEQ ID NOS: 103976, 103977, 103978, 103979, 103980, 103981, 103982, 103983, 103984, 103985, 103986 were selected from the ORFs having the nucleotide sequence represented by SEQ ID NO: 77240.

The probes having the nucleotide sequence represented by SEQ ID NOS: 103987, 103988, 103989, 103990, 103991, 103992, 103993, 103994, 103995, 103996, 103997 were selected from the ORFs having the nucleotide sequence represented by SEQ ID NO: 77241.

The probes having the nucleotide sequence represented by SEQ ID NOS: 103998, 103999, 104000, 104001, 104002, 104003, 104004, 104005, 104006, 104007, 104008 were selected from the ORFs having the nucleotide sequence represented by SEQ ID NO: 77242.

The probes having the nucleotide sequence represented by SEQ ID NOS: 104009, 104010, 104011, 104012, 104013, 104014, 104015, 104016, 104017, 104018, 104019 were selected from the ORFs having the nucleotide sequence represented by SEQ ID NO: 77243.

The probes having the nucleotide sequence represented by SEQ ID NOS: 104020, 104021, 104022, 104023, 104024, 104025, 104026, 104027, 104028, 104029, 104030 were selected from the ORFs having the nucleotide sequence represented by SEQ ID NO: 77244.

The probes having the nucleotide sequence represented by SEQ ID NOS: 104031, 104032, 104033, 104034, 104035, 104036, 104037, 104038, 104039, 104040, 104041 were selected from the ORFs having the nucleotide sequence represented by SEQ ID NO: 77245.

The probes having the nucleotide sequence represented by SEQ ID NOS: 104042, 104043, 104044, 104045, 104046, 104047, 104048, 104049, 104050, 104051, 104052 were selected from the ORFs having the nucleotide sequence represented by SEQ ID NO: 77246.

The probes having the nucleotide sequence represented by SEQ ID NOS: 104053, 104054, 104055, 104056, 104057, 104058, 104059, 104060, 104061, 104062, 104063 were selected from the ORFs having the nucleotide sequence represented by SEQ ID NO: 77247.

The probes having the nucleotide sequence represented by SEQ ID NOS: 104064, 104065, 104066, 104067, 104068, 104069, 104070, 104071, 104072, 104073, 104074 were selected from the ORFs having the nucleotide sequence represented by SEQ ID NO: 77248.

The probes having the nucleotide sequence represented by SEQ ID NOS: 104075, 104076, 104077, 104078, 104079, 104080, 104081, 104082, 104083, 104084, 104085 were selected from the ORFs having the nucleotide sequence represented by SEQ ID NO: 77249.

The probes having the nucleotide sequence represented by SEQ ID NOS: 104086, 104087, 104088, 104089, 104090, 104091, 104092, 104093, 104094, 104095, 104096 were selected from the ORFs having the nucleotide sequence represented by SEQ ID NO: 77250.

The probes having the nucleotide sequence represented by SEQ ID NOS: 104097, 104098, 104099, 104100, 104101, 104102, 104103, 104104, 104105, 104106, 104107 were selected from the ORFs having the nucleotide sequence represented by SEQ ID NO: 77251.

The probes having the nucleotide sequence represented by SEQ ID NOS: 104108, 104109, 104110, 104111, 104112, 104113, 104114, 104115, 104116, 104117, 104118 were selected from the ORFs having the nucleotide sequence represented by SEQ ID NO: 77252.

The probes having the nucleotide sequence represented by SEQ ID NOS: 104119, 104120, 104121, 104122, 104123, 104124, 104125, 104126, 104127, 104128, 104129 were selected from the ORFs having the nucleotide sequence represented by SEQ ID NO: 77253.

The probes having the nucleotide sequence represented by SEQ ID NOS: 104130, 104131, 104132, 104133, 104134, 104135, 104136, 104137, 104138, 104139, 104140 were selected from the ORFs having the nucleotide sequence represented by SEQ ID NO: 77254.

The probes having the nucleotide sequence represented by SEQ ID NOS: 104141, 104142, 104143, 104144, 104145, 104146, 104147, 104148, 104149, 104150, 104151 were selected from the ORFs having the nucleotide sequence represented by SEQ ID NO: 77255.

The probes having the nucleotide sequence represented by SEQ ID NOS: 104152, 104153, 104154, 104155, 104156, 104157, 104158, 104159, 104160, 104161, 104162 were selected from the ORFs having the nucleotide sequence represented by SEQ ID NO: 77256.

The probes having the nucleotide sequence represented by SEQ ID NOS: 104163, 104164, 104165, 104166, 104167, 104168, 104169, 104170, 104171, 104172, 104173 were selected from the ORFs having the nucleotide sequence represented by SEQ ID NO: 77257.

The probes having the nucleotide sequence represented by SEQ ID NOS: 104174, 104175, 104176, 104177, 104178, 104179, 104180, 104181, 104182, 104183, 104184 were selected from the ORFs having the nucleotide sequence represented by SEQ ID NO: 77258.

The probes having the nucleotide sequence represented by SEQ ID NOS: 104185, 104186, 104187, 104188, 104189, 104190, 104191, 104192, 104193, 104194, 104195 were selected from the ORFs having the nucleotide sequence represented by SEQ ID NO: 77259.

The probes having the nucleotide sequence represented by SEQ ID NOS: 104196, 104197, 104198, 104199, 104200, 104201, 104202, 104203, 104204, 104205, 104206 were selected from the ORFs having the nucleotide sequence represented by SEQ ID NO: 77260.

The probes having the nucleotide sequence represented by SEQ ID NOS: 104207, 104208, 104209, 104210, 104211, 104212, 104213, 104214, 104215, 104216, 104217 were selected from the ORFs having the nucleotide sequence represented by SEQ ID NO: 77261.

The probes having the nucleotide sequence represented by SEQ ID NOS: 104218, 104219, 104220, 104221, 104222, 104223, 104224, 104225, 104226, 104227, 104228 were selected from the ORFs having the nucleotide sequence represented by SEQ ID NO: 77262.

The probes having the nucleotide sequence represented by SEQ ID NOS: 104229, 104230, 104231, 104232, 104233, 104234, 104235, 104236, 104237, 104238, 104239 were selected from the ORFs having the nucleotide sequence represented by SEQ ID NO: 77263.

The probes having the nucleotide sequence represented by SEQ ID NOS: 104240, 104241, 104242, 104243, 104244, 104245, 104246, 104247, 104248, 104249, 104250 were selected from the ORFs having the nucleotide sequence represented by SEQ ID NO: 77264.

The probes having the nucleotide sequence represented by SEQ ID NOS: 104251, 104252, 104253, 104254, 104255, 104256, 104257, 104258, 104259, 104260, 104261 were selected from the ORFs having the nucleotide sequence represented by SEQ ID NO: 77265.

The probes having the nucleotide sequence represented by SEQ ID NOS: 104262, 104263, 104264, 104265, 104266, 104267, 104268, 104269, 104270, 104271, 104272 were selected from the ORFs having the nucleotide sequence represented by SEQ ID NO: 77266.

The probes having the nucleotide sequence represented by SEQ ID NOS: 104273, 104274, 104275, 104276, 104277, 104278, 104279, 104280, 104281, 104282, 104283 were selected from the ORFs having the nucleotide sequence represented by SEQ ID NO: 77267.

The probes having the nucleotide sequence represented by SEQ ID NOS: 104284, 104285, 104286, 104287, 104288, 104289, 104290, 104291, 104292, 104293, 104294 were selected from the ORFs having the nucleotide sequence represented by SEQ ID NO: 77268.

The probes having the nucleotide sequence represented by SEQ ID NOS: 104295, 104296, 104297, 104298, 104299, 104300, 104301, 104302, 104303, 104304, 104305 were selected from the ORFs having the nucleotide sequence represented by SEQ ID NO: 77269.

The probes having the nucleotide sequence represented by SEQ ID NOS: 104306, 104307, 104308, 104309, 104310, 104311, 104312, 104313, 104314, 104315, 104316 were selected from the ORFs having the nucleotide sequence represented by SEQ ID NO: 77270.

The probes having the nucleotide sequence represented by SEQ ID NOS: 104317, 104318, 104319, 104320, 104321, 104322, 104323, 104324, 104325, 104326, 104327 were selected from the ORFs having the nucleotide sequence represented by SEQ ID NO: 77271.

The probes having the nucleotide sequence represented by SEQ ID NOS: 104328, 104329, 104330, 104331, 104332, 104333, 104334, 104335, 104336, 104337, 104338 were selected from the ORFs having the nucleotide sequence represented by SEQ ID NO: 77272.

The probes having the nucleotide sequence represented by SEQ ID NOS: 104339, 104340, 104341, 104342, 104343, 104344, 104345, 104346, 104347, 104348, 104349 were selected from the ORFs having the nucleotide sequence represented by SEQ ID NO: 77273.

The probes having the nucleotide sequence represented by SEQ ID NOS: 104350, 104351, 104352, 104353, 104354, 104355, 104356, 104357, 104358, 104359, 104360 were selected from the ORFs having the nucleotide sequence represented by SEQ ID NO: 77274.

The probes having the nucleotide sequence represented by SEQ ID NOS: 104361, 104362, 104363, 104364, 104365, 104366, 104367, 104368, 104369, 104370, 104371 were selected from the ORFs having the nucleotide sequence represented by SEQ ID NO: 77275.

The probes having the nucleotide sequence represented by SEQ ID NOS: 104372, 104373, 104374, 104375, 104376, 104377, 104378, 104379, 104380, 104381, 104382 were selected from the ORFs having the nucleotide sequence represented by SEQ ID NO: 77276.

The probes having the nucleotide sequence represented by SEQ ID NOS: 104383, 104384, 104385, 104386, 104387, 104388, 104389, 104390, 104391, 104392, 104393 were selected from the ORFs having the nucleotide sequence represented by SEQ ID NO: 77277.

The probes having the nucleotide sequence represented by SEQ ID NOS: 104394, 104395, 104396, 104397, 104398, 104399, 104400, 104401, 104402, 104403, 104404 were selected from the ORFs having the nucleotide sequence represented by SEQ ID NO: 77278.

The probes having the nucleotide sequence represented by SEQ ID NOS: 104405, 104406, 104407, 104408, 104409, 104410, 104411, 104412, 104413, 104414, 104415 were selected from the ORFs having the nucleotide sequence represented by SEQ ID NO: 77279.

The probes having the nucleotide sequence represented by SEQ ID NOS: 104416, 104417, 104418, 104419, 104420, 104421, 104422, 104423, 104424, 104425, 104426 were selected from the ORFs having the nucleotide sequence represented by SEQ ID NO: 77280.

The probes having the nucleotide sequence represented by SEQ ID NOS: 104427, 104428, 104429, 104430, 104431, 104432, 104433, 104434, 104435, 104436, 104437 were selected from the ORFs having the nucleotide sequence represented by SEQ ID NO: 77281.

The probes having the nucleotide sequence represented by SEQ ID NOS: 104438, 104439, 104440, 104441, 104442, 104443, 104444, 104445, 104446, 104447, 104448 were selected from the ORFs having the nucleotide sequence represented by SEQ ID NO: 77282.

The probes having the nucleotide sequence represented by SEQ ID NOS: 104449, 104450, 104451, 104452, 104453, 104454, 104455, 104456, 104457, 104458, 104459 were selected from the ORFs having the nucleotide sequence represented by SEQ ID NO: 77283.

The probes having the nucleotide sequence represented by SEQ ID NOS: 104460, 104461, 104462, 104463, 104464, 104465, 104466, 104467, 104468, 104469, 104470 were selected from the ORFs having the nucleotide sequence represented by SEQ ID NO: 77284.

The probes having the nucleotide sequence represented by SEQ ID NOS: 104471, 104472, 104473, 104474, 104475, 104476, 104477, 104478, 104479, 104480, 104481 were selected from the ORFs having the nucleotide sequence represented by SEQ ID NO: 77285.

The probes having the nucleotide sequence represented by SEQ ID NOS: 104482, 104483, 104484, 104485, 104486, 104487, 104488, 104489, 104490, 104491, 104492 were selected from the ORFs having the nucleotide sequence represented by SEQ ID NO: 77286.

The probes having the nucleotide sequence represented by SEQ ID NOS: 104493, 104494, 104495, 104496, 104497, 104498, 104499, 104500, 104501, 104502, 104503 were selected from the ORFs having the nucleotide sequence represented by SEQ ID NO: 77287.

The probes having the nucleotide sequence represented by SEQ ID NOS: 104504, 104505, 104506, 104507, 104508, 104509, 104510, 104511, 104512, 104513, 104514 were selected from the ORFs having the nucleotide sequence represented by SEQ ID NO: 77288.

The probes having the nucleotide sequence represented by SEQ ID NOS: 104515, 104516, 104517, 104518, 104519, 104520, 104521, 104522, 104523, 104524, 104525 were selected from the ORFs having the nucleotide sequence represented by SEQ ID NO: 77289.

The probes having the nucleotide sequence represented by SEQ ID NOS: 104526, 104527, 104528, 104529, 104530, 104531, 104532, 104533, 104534, 104535, 104536 were selected from the ORFs having the nucleotide sequence represented by SEQ ID NO: 77290.

The probes having the nucleotide sequence represented by SEQ ID NOS: 104537, 104538, 104539, 104540, 104541, 104542, 104543, 104544, 104545, 104546, 104547 were selected from the ORFs having the nucleotide sequence represented by SEQ ID NO: 77291.

The probes having the nucleotide sequence represented by SEQ ID NOS: 104548, 104549, 104550, 104551, 104552, 104553, 104554, 104555, 104556, 104557, 104558 were selected from the ORFs having the nucleotide sequence represented by SEQ ID NO: 77292.

The probes having the nucleotide sequence represented by SEQ ID NOS: 104559, 104560, 104561, 104562, 104563, 104564, 104565, 104566, 104567, 104568, 104569 were selected from the ORFs having the nucleotide sequence represented by SEQ ID NO: 77293.

The probes having the nucleotide sequence represented by SEQ ID NOS: 104570, 104571, 104572, 104573, 104574, 104575, 104576, 104577, 104578, 104579, 104580 were selected from the ORFs having the nucleotide sequence represented by SEQ ID NO: 77294.

The probes having the nucleotide sequence represented by SEQ ID NOS: 104581, 104582, 104583, 104584, 104585, 104586, 104587, 104588, 104589, 104590, 104591 were selected from the ORFs having the nucleotide sequence represented by SEQ ID NO: 77295.

The probes having the nucleotide sequence represented by SEQ ID NOS: 104592, 104593, 104594, 104595, 104596, 104597, 104598, 104599, 104600, 104601, 104602 were selected from the ORFs having the nucleotide sequence represented by SEQ ID NO: 77296.

The probes having the nucleotide sequence represented by SEQ ID NOS: 104603, 104604, 104605, 104606, 104607, 104608, 104609, 104610, 104611, 104612, 104613 were selected from the ORFs having the nucleotide sequence represented by SEQ ID NO: 77297.

The probes having the nucleotide sequence represented by SEQ ID NOS: 104614, 104615, 104616, 104617, 104618, 104619, 104620, 104621, 104622, 104623, 104624 were selected from the ORFs having the nucleotide sequence represented by SEQ ID NO: 77298.

The probes having the nucleotide sequence represented by SEQ ID NOS: 104625, 104626, 104627, 104628, 104629, 104630, 104631, 104632, 104633, 104634, 104635 were selected from the ORFs having the nucleotide sequence represented by SEQ ID NO: 77299.

The probes having the nucleotide sequence represented by SEQ ID NOS: 104636, 104637, 104638, 104639, 104640, 104641, 104642, 104643, 104644, 104645, 104646 were selected from the ORFs having the nucleotide sequence represented by SEQ ID NO: 77300.

The probes having the nucleotide sequence represented by SEQ ID NOS: 104647, 104648, 104649, 104650, 104651, 104652, 104653, 104654, 104655, 104656, 104657 were selected from the ORFs having the nucleotide sequence represented by SEQ ID NO: 77301.

The probes having the nucleotide sequence represented by SEQ ID NOS: 104658, 104659, 104660, 104661, 104662, 104663, 104664, 104665, 104666, 104667, 104668 were selected from the ORFs having the nucleotide sequence represented by SEQ ID NO: 77302.

The probes having the nucleotide sequence represented by SEQ ID NOS: 104669, 104670, 104671, 104672, 104673, 104674, 104675, 104676, 104677, 104678, 104679 were selected from the ORFs having the nucleotide sequence represented by SEQ ID NO: 77303.

The probes having the nucleotide sequence represented by SEQ ID NOS: 104680, 104681, 104682, 104683, 104684, 104685, 104686, 104687, 104688, 104689, 104690 were selected from the ORFs having the nucleotide sequence represented by SEQ ID NO: 77304.

The probes having the nucleotide sequence represented by SEQ ID NOS: 104691, 104692, 104693, 104694, 104695, 104696, 104697, 104698, 104699, 104700, 104701 were selected from the ORFs having the nucleotide sequence represented by SEQ ID NO: 77305.

The probes having the nucleotide sequence represented by SEQ ID NOS: 104702, 104703, 104704, 104705, 104706, 104707, 104708, 104709, 104710, 104711, 104712 were selected from the ORFs having the nucleotide sequence represented by SEQ ID NO: 77306.

The probes having the nucleotide sequence represented by SEQ ID NOS: 104713, 104714, 104715, 104716, 104717, 104718, 104719, 104720, 104721, 104722, 104723 were selected from the ORFs having the nucleotide sequence represented by SEQ ID NO: 77307.

The probes having the nucleotide sequence represented by SEQ ID NOS: 104724, 104725, 104726, 104727, 104728, 104729, 104730, 104731, 104732, 104733, 104734 were selected from the ORFs having the nucleotide sequence represented by SEQ ID NO: 77308.

The probes having the nucleotide sequence represented by SEQ ID NOS: 104735, 104736, 104737, 104738, 104739, 104740, 104741, 104742, 104743, 104744, 104745 were selected from the ORFs having the nucleotide sequence represented by SEQ ID NO: 77309.

The probes having the nucleotide sequence represented by SEQ ID NOS: 104746, 104747, 104748, 104749, 104750, 104751, 104752, 104753, 104754, 104755, 104756 were selected from the ORFs having the nucleotide sequence represented by SEQ ID NO: 77310.

The probes having the nucleotide sequence represented by SEQ ID NOS: 104757, 104758, 104759, 104760, 104761, 104762, 104763, 104764, 104765, 104766, 104767 were selected from the ORFs having the nucleotide sequence represented by SEQ ID NO: 77311.

The probes having the nucleotide sequence represented by SEQ ID NOS: 104768, 104769, 104770, 104771, 104772, 104773, 104774, 104775, 104776, 104777, 104778 were selected from the ORFs having the nucleotide sequence represented by SEQ ID NO: 77312.

The probes having the nucleotide sequence represented by SEQ ID NOS: 104779, 104780, 104781, 104782, 104783, 104784, 104785, 104786, 104787, 104788, 104789 were selected from the ORFs having the nucleotide sequence represented by SEQ ID NO: 77313.

The probes having the nucleotide sequence represented by SEQ ID NOS: 104790, 104791, 104792, 104793, 104794, 104795, 104796, 104797, 104798, 104799, 104800 were selected from the ORFs having the nucleotide sequence represented by SEQ ID NO: 77314.

The probes having the nucleotide sequence represented by SEQ ID NOS: 104801, 104802, 104803, 104804, 104805, 104806, 104807, 104808, 104809, 104810, 104811 were selected from the ORFs having the nucleotide sequence represented by SEQ ID NO: 77315.

The probes having the nucleotide sequence represented by SEQ ID NOS: 104812, 104813, 104814, 104815, 104816, 104817, 104818, 104819, 104820, 104821, 104822 were selected from the ORFs having the nucleotide sequence represented by SEQ ID NO: 77316.

The probes having the nucleotide sequence represented by SEQ ID NOS: 104823, 104824, 104825, 104826, 104827, 104828, 104829, 104830, 104831, 104832, 104833 were selected from the ORFs having the nucleotide sequence represented by SEQ ID NO: 77317.

The probes having the nucleotide sequence represented by SEQ ID NOS: 104834, 104835, 104836, 104837, 104838, 104839, 104840, 104841, 104842, 104843, 104844 were selected from the ORFs having the nucleotide sequence represented by SEQ ID NO: 77318.

The probes having the nucleotide sequence represented by SEQ ID NOS: 104845, 104846, 104847, 104848, 104849, 104850, 104851, 104852, 104853, 104854, 104855 were selected from the ORFs having the nucleotide sequence represented by SEQ ID NO: 77319.

The probes having the nucleotide sequence represented by SEQ ID NOS: 104856, 104857, 104858, 104859, 104860, 104861, 104862, 104863, 104864, 104865, 104866 were selected from the ORFs having the nucleotide sequence represented by SEQ ID NO: 77320.

The probes having the nucleotide sequence represented by SEQ ID NOS: 104867, 104868, 104869, 104870, 104871, 104872, 104873, 104874, 104875, 104876, 104877 were selected from the ORFs having the nucleotide sequence represented by SEQ ID NO: 77321.

The probes having the nucleotide sequence represented by SEQ ID NOS: 104878, 104879, 104880, 104881, 104882, 104883, 104884, 104885, 104886, 104887, 104888 were selected from the ORFs having the nucleotide sequence represented by SEQ ID NO: 77322.

The probes having the nucleotide sequence represented by SEQ ID NOS: 104889, 104890, 104891, 104892, 104893, 104894, 104895, 104896, 104897, 104898, 104899 were selected from the ORFs having the nucleotide sequence represented by SEQ ID NO: 77323.

The probes having the nucleotide sequence represented by SEQ ID NOS: 104900, 104901, 104902, 104903, 104904, 104905, 104906, 104907, 104908, 104909, 104910 were selected from the ORFs having the nucleotide sequence represented by SEQ ID NO: 77324.

The probes having the nucleotide sequence represented by SEQ ID NOS: 104911, 104912, 104913, 104914, 104915, 104916, 104917, 104918, 104919, 104920, 104921 were selected from the ORFs having the nucleotide sequence represented by SEQ ID NO: 77325.

The probes having the nucleotide sequence represented by SEQ ID NOS: 104922, 104923, 104924, 104925, 104926, 104927, 104928, 104929, 104930, 104931, 104932 were selected from the ORFs having the nucleotide sequence represented by SEQ ID NO: 77326.

The probes having the nucleotide sequence represented by SEQ ID NOS: 104933, 104934, 104935, 104936, 104937, 104938, 104939, 104940, 104941, 104942, 104943 were selected from the ORFs having the nucleotide sequence represented by SEQ ID NO: 77327.

The probes having the nucleotide sequence represented by SEQ ID NOS: 104944, 104945, 104946, 104947, 104948, 104949, 104950, 104951, 104952, 104953, 104954 were selected from the ORFs having the nucleotide sequence represented by SEQ ID NO: 77328.

The probes having the nucleotide sequence represented by SEQ ID NOS: 104955, 104956, 104957, 104958, 104959, 104960, 104961, 104962, 104963, 104964, 104965 were selected from the ORFs having the nucleotide sequence represented by SEQ ID NO: 77329.

The probes having the nucleotide sequence represented by SEQ ID NOS: 104966, 104967, 104968, 104969, 104970, 104971, 104972, 104973, 104974, 104975, 104976 were selected from the ORFs having the nucleotide sequence represented by SEQ ID NO: 77330.

The probes having the nucleotide sequence represented by SEQ ID NOS: 104977, 104978, 104979, 104980, 104981, 104982, 104983, 104984, 104985, 104986, 104987 were selected from the ORFs having the nucleotide sequence represented by SEQ ID NO: 77331.

The probes having the nucleotide sequence represented by SEQ ID NOS: 104988, 104989, 104990, 104991, 104992, 104993, 104994, 104995, 104996, 104997, 104998 were selected from the ORFs having the nucleotide sequence represented by SEQ ID NO: 77332.

The probes having the nucleotide sequence represented by SEQ ID NOS: 104999, 105000, 105001, 105002, 105003, 105004, 105005, 105006, 105007, 105008, 105009 were selected from the ORFs having the nucleotide sequence represented by SEQ ID NO: 77333.

The probes having the nucleotide sequence represented by SEQ ID NOS: 105010, 105011, 105012, 105013, 105014, 105015, 105016, 105017, 105018, 105019, 105020 were selected from the ORFs having the nucleotide sequence represented by SEQ ID NO: 77334.

The probes having the nucleotide sequence represented by SEQ ID NOS: 105021, 105022, 105023, 105024, 105025, 105026, 105027, 105028, 105029, 105030, 105031 were selected from the ORFs having the nucleotide sequence represented by SEQ ID NO: 77335.

The probes having the nucleotide sequence represented by SEQ ID NOS: 105032, 105033, 105034, 105035, 105036, 105037, 105038, 105039, 105040, 105041, 105042 were selected from the ORFs having the nucleotide sequence represented by SEQ ID NO: 77336.

The probes having the nucleotide sequence represented by SEQ ID NOS: 105043, 105044, 105045, 105046, 105047, 105048, 105049, 105050, 105051, 105052, 105053 were selected from the ORFs having the nucleotide sequence represented by SEQ ID NO: 77337.

The probes having the nucleotide sequence represented by SEQ ID NOS: 105054, 105055, 105056, 105057, 105058, 105059, 105060, 105061, 105062, 105063, 105064 were selected from the ORFs having the nucleotide sequence represented by SEQ ID NO: 77338.

The probes having the nucleotide sequence represented by SEQ ID NOS: 105065, 105066, 105067, 105068, 105069, 105070, 105071, 105072, 105073, 105074, 105075 were selected from the ORFs having the nucleotide sequence represented by SEQ ID NO: 77339.

The probes having the nucleotide sequence represented by SEQ ID NOS: 105076, 105077, 105078, 105079, 105080, 105081, 105082, 105083, 105084, 105085, 105086 were selected from the ORFs having the nucleotide sequence represented by SEQ ID NO: 77340.

The probes having the nucleotide sequence represented by SEQ ID NOS: 105087, 105088, 105089, 105090, 105091, 105092, 105093, 105094, 105095, 105096, 105097 were selected from the ORFs having the nucleotide sequence represented by SEQ ID NO: 77341.

The probes having the nucleotide sequence represented by SEQ ID NOS: 105098, 105099, 105100, 105101, 105102, 105103, 105104, 105105, 105106, 105107, 105108 were selected from the ORFs having the nucleotide sequence represented by SEQ ID NO: 77342.

The probes having the nucleotide sequence represented by SEQ ID NOS: 105109, 105110, 105111, 105112, 105113, 105114, 105115, 105116, 105117, 105118, 105119 were selected from the ORFs having the nucleotide sequence represented by SEQ ID NO: 77343.

The probes having the nucleotide sequence represented by SEQ ID NOS: 105120, 105121, 105122, 105123, 105124, 105125, 105126, 105127, 105128, 105129, 105130 were selected from the ORFs having the nucleotide sequence represented by SEQ ID NO: 77344.

The probes having the nucleotide sequence represented by SEQ ID NOS: 105131, 105132, 105133, 105134, 105135, 105136, 105137, 105138, 105139, 105140, 105141 were selected from the ORFs having the nucleotide sequence represented by SEQ ID NO: 77345.

The probes having the nucleotide sequence represented by SEQ ID NOS: 105142, 105143, 105144, 105145, 105146, 105147, 105148, 105149, 105150, 105151, 105152 were selected from the ORFs having the nucleotide sequence represented by SEQ ID NO: 77346.

The probes having the nucleotide sequence represented by SEQ ID NOS: 105153, 105154, 105155, 105156, 105157, 105158, 105159, 105160, 105161, 105162, 105163 were selected from the ORFs having the nucleotide sequence represented by SEQ ID NO: 77347.

The probes having the nucleotide sequence represented by SEQ ID NOS: 105164, 105165, 105166, 105167, 105168, 105169, 105170, 105171, 105172, 105173, 105174 were selected from the ORFs having the nucleotide sequence represented by SEQ ID NO: 77348.

The probes having the nucleotide sequence represented by SEQ ID NOS: 105175, 105176, 105177, 105178, 105179, 105180, 105181, 105182, 105183, 105184, 105185, 105186, 105187, 105188, 105189, 105190, 105191, 105192, 105193, 105194, 105195, 105196 were selected from the ORFs having the nucleotide sequence represented by SEQ ID NO: 77349.

The probes having the nucleotide sequence represented by SEQ ID NOS: 105197, 105198, 105199, 105200, 105201, 105202, 105203, 105204, 105205, 105206, 105207 were selected from the ORFs having the nucleotide sequence represented by SEQ ID NO: 77350.

The probes having the nucleotide sequence represented by SEQ ID NOS: 105208, 105209, 105210, 105211, 105212, 105213, 105214, 105215, 105216, 105217, 105218 were selected from the ORFs having the nucleotide sequence represented by SEQ ID NO: 77351.

The probes having the nucleotide sequence represented by SEQ ID NOS: 105219, 105220, 105221, 105222, 105223, 105224, 105225, 105226, 105227, 105228, 105229 were selected from the ORFs having the nucleotide sequence represented by SEQ ID NO: 77352.

The probes having the nucleotide sequence represented by SEQ ID NOS: 105230, 105231, 105232, 105233, 105234, 105235, 105236, 105237, 105238, 105239, 105240 were selected from the ORFs having the nucleotide sequence represented by SEQ ID NO: 77353.

The probes having the nucleotide sequence represented by SEQ ID NOS: 105241, 105242, 105243, 105244, 105245, 105246, 105247, 105248, 105249, 105250, 105251 were selected from the ORFs having the nucleotide sequence represented by SEQ ID NO: 77354.

The probes having the nucleotide sequence represented by SEQ ID NOS: 105252, 105253, 105254, 105255, 105256, 105257, 105258, 105259, 105260, 105261, 105262 were selected from the ORFs having the nucleotide sequence represented by SEQ ID NO: 77355.

The probes having the nucleotide sequence represented by SEQ ID NOS: 105263, 105264, 105265, 105266, 105267, 105268, 105269, 105270, 105271, 105272, 105273 were selected from the ORFs having the nucleotide sequence represented by SEQ ID NO: 77356.

The probes having the nucleotide sequence represented by SEQ ID NOS: 105274, 105275, 105276, 105277, 105278, 105279, 105280, 105281, 105282, 105283, 105284 were selected from the ORFs having the nucleotide sequence represented by SEQ ID NO: 77357.

The probes having the nucleotide sequence represented by SEQ ID NOS: 105285, 105286, 105287, 105288, 105289, 105290, 105291, 105292, 105293, 105294, 105295 were selected from the ORFs having the nucleotide sequence represented by SEQ ID NO: 77358.

The probes having the nucleotide sequence represented by SEQ ID NOS: 105296, 105297, 105298, 105299, 105300, 105301, 105302, 105303, 105304, 105305, 105306 were selected from the ORFs having the nucleotide sequence represented by SEQ ID NO: 77359.

The probes having the nucleotide sequence represented by SEQ ID NOS: 105307, 105308, 105309, 105310, 105311, 105312, 105313, 105314, 105315, 105316, 105317 were selected from the ORFs having the nucleotide sequence represented by SEQ ID NO: 77360.

The probes having the nucleotide sequence represented by SEQ ID NOS: 105318, 105319, 105320, 105321, 105322, 105323, 105324, 105325, 105326, 105327, 105328 were selected from the ORFs having the nucleotide sequence represented by SEQ ID NO: 77361.

The probes having the nucleotide sequence represented by SEQ ID NOS: 105329, 105330, 105331, 105332, 105333, 105334, 105335, 105336, 105337, 105338, 105339 were selected from the ORFs having the nucleotide sequence represented by SEQ ID NO: 77362.

The probes having the nucleotide sequence represented by SEQ ID NOS: 105340, 105341, 105342, 105343, 105344, 105345, 105346, 105347, 105348, 105349, 105350 were selected from the ORFs having the nucleotide sequence represented by SEQ ID NO: 77363.

The probes having the nucleotide sequence represented by SEQ ID NOS: 105351, 105352, 105353, 105354, 105355, 105356, 105357, 105358, 105359, 105360, 105361 were selected from the ORFs having the nucleotide sequence represented by SEQ ID NO: 77364.

The probes having the nucleotide sequence represented by SEQ ID NOS: 105362, 105363, 105364, 105365, 105366, 105367, 105368, 105369, 105370, 105371, 105372 were selected from the ORFs having the nucleotide sequence represented by SEQ ID NO: 77365.

The probes having the nucleotide sequence represented by SEQ ID NOS: 105373, 105374, 105375, 105376, 105377, 105378, 105379, 105380, 105381, 105382, 105383 were selected from the ORFs having the nucleotide sequence represented by SEQ ID NO: 77366.

The probes having the nucleotide sequence represented by SEQ ID NOS: 105384, 105385, 105386, 105387, 105388, 105389, 105390, 105391, 105392, 105393, 105394 were selected from the ORFs having the nucleotide sequence represented by SEQ ID NO: 77367.

The probes having the nucleotide sequence represented by SEQ ID NOS: 105395, 105396, 105397, 105398, 105399, 105400, 105401, 105402, 105403, 105404, 105405 were selected from the ORFs having the nucleotide sequence represented by SEQ ID NO: 77368.

The probes having the nucleotide sequence represented by SEQ ID NOS: 105406, 105407, 105408, 105409, 105410, 105411, 105412, 105413, 105414, 105415, 105416 were selected from the ORFs having the nucleotide sequence represented by SEQ ID NO: 77369.

The probes having the nucleotide sequence represented by SEQ ID NOS: 105417, 105418, 105419, 105420, 105421, 105422, 105423, 105424, 105425, 105426, 105427 were selected from the ORFs having the nucleotide sequence represented by SEQ ID NO: 77370.

The probes having the nucleotide sequence represented by SEQ ID NOS: 105428, 105429, 105430, 105431, 105432, 105433, 105434, 105435, 105436, 105437, 105438 were selected from the ORFs having the nucleotide sequence represented by SEQ ID NO: 77371.

The probes having the nucleotide sequence represented by SEQ ID NOS: 105439, 105440, 105441, 105442, 105443, 105444, 105445, 105446, 105447, 105448, 105449 were selected from the ORFs having the nucleotide sequence represented by SEQ ID NO: 77372.

The probes having the nucleotide sequence represented by SEQ ID NOS: 105450, 105451, 105452, 105453, 105454, 105455, 105456, 105457, 105458, 105459, 105460 were selected from the ORFs having the nucleotide sequence represented by SEQ ID NO: 77373.

The probes having the nucleotide sequence represented by SEQ ID NOS: 105461, 105462, 105463, 105464, 105465, 105466, 105467, 105468, 105469, 105470, 105471 were selected from the ORFs having the nucleotide sequence represented by SEQ ID NO: 77374.

The probes having the nucleotide sequence represented by SEQ ID NOS: 105472, 105473, 105474, 105475, 105476, 105477, 105478, 105479, 105480, 105481, 105482 were selected from the ORFs having the nucleotide sequence represented by SEQ ID NO: 77375.

The probes having the nucleotide sequence represented by SEQ ID NOS: 105483, 105484, 105485, 105486, 105487, 105488, 105489, 105490, 105491, 105492, 105493 were selected from the ORFs having the nucleotide sequence represented by SEQ ID NO: 77376.

The probes having the nucleotide sequence represented by SEQ ID NOS: 105494, 105495, 105496, 105497, 105498, 105499, 105500, 105501, 105502, 105503, 105504 were selected from the ORFs having the nucleotide sequence represented by SEQ ID NO: 77377.

The probes having the nucleotide sequence represented by SEQ ID NOS: 105505, 105506, 105507, 105508, 105509, 105510, 105511, 105512, 105513, 105514, 105515 were selected from the ORFs having the nucleotide sequence represented by SEQ ID NO: 77378.

The probes having the nucleotide sequence represented by SEQ ID NOS: 105516, 105517, 105518, 105519, 105520, 105521, 105522, 105523, 105524, 105525, 105526 were selected from the ORFs having the nucleotide sequence represented by SEQ ID NO: 77379.

The probes having the nucleotide sequence represented by SEQ ID NOS: 105527, 105528, 105529, 105530, 105531, 105532, 105533, 105534, 105535, 105536, 105537 were selected from the ORFs having the nucleotide sequence represented by SEQ ID NO: 77380.

The probes having the nucleotide sequence represented by SEQ ID NOS: 105538, 105539, 105540, 105541, 105542, 105543, 105544, 105545, 105546, 105547, 105548 were selected from the ORFs having the nucleotide sequence represented by SEQ ID NO: 77381.

The probes having the nucleotide sequence represented by SEQ ID NOS: 105549, 105550, 105551, 105552, 105553, 105554, 105555, 105556, 105557, 105558, 105559 were selected from the ORFs having the nucleotide sequence represented by SEQ ID NO: 77382.

The probes having the nucleotide sequence represented by SEQ ID NOS: 105560, 105561, 105562, 105563, 105564, 105565, 105566, 105567, 105568, 105569, 105570 were selected from the ORFs having the nucleotide sequence represented by SEQ ID NO: 77383.

The probes having the nucleotide sequence represented by SEQ ID NOS: 105571, 105572, 105573, 105574, 105575, 105576, 105577, 105578, 105579, 105580, 105581 were selected from the ORFs having the nucleotide sequence represented by SEQ ID NO: 77384.

The probes having the nucleotide sequence represented by SEQ ID NOS: 105582, 105583, 105584, 105585, 105586, 105587, 105588, 105589, 105590, 105591, 105592 were selected from the ORFs having the nucleotide sequence represented by SEQ ID NO: 77385.

The probes having the nucleotide sequence represented by SEQ ID NOS: 105593, 105594, 105595, 105596, 105597, 105598, 105599, 105600, 105601, 105602, 105603 were selected from the ORFs having the nucleotide sequence represented by SEQ ID NO: 77386.

The probes having the nucleotide sequence represented by SEQ ID NOS: 105604, 105605, 105606, 105607, 105608, 105609, 105610, 105611, 105612, 105613, 105614 were selected from the ORFs having the nucleotide sequence represented by SEQ ID NO: 77387.

The probes having the nucleotide sequence represented by SEQ ID NOS: 105615, 105616, 105617, 105618, 105619, 105620, 105621, 105622, 105623, 105624, 105625 were selected from the ORFs having the nucleotide sequence represented by SEQ ID NO: 77388.

The probes having the nucleotide sequence represented by SEQ ID NOS: 105626, 105627, 105628, 105629, 105630, 105631, 105632, 105633, 105634, 105635, 105636 were selected from the ORFs having the nucleotide sequence represented by SEQ ID NO: 77389.

The probes having the nucleotide sequence represented by SEQ ID NOS: 105637, 105638, 105639, 105640, 105641, 105642, 105643, 105644, 105645, 105646, 105647 were selected from the ORFs having the nucleotide sequence represented by SEQ ID NO: 77390.

The probes having the nucleotide sequence represented by SEQ ID NOS: 105648, 105649, 105650, 105651, 105652, 105653, 105654, 105655, 105656, 105657, 105658 were selected from the ORFs having the nucleotide sequence represented by SEQ ID NO: 77391.

The probes having the nucleotide sequence represented by SEQ ID NOS: 105659, 105660, 105661, 105662, 105663, 105664, 105665, 105666, 105667, 105668, 105669 were selected from the ORFs having the nucleotide sequence represented by SEQ ID NO: 77392.

The probes having the nucleotide sequence represented by SEQ ID NOS: 105670, 105671, 105672, 105673, 105674, 105675, 105676, 105677, 105678, 105679, 105680 were selected from the ORFs having the nucleotide sequence represented by SEQ ID NO: 77393.

The probes having the nucleotide sequence represented by SEQ ID NOS: 105681, 105682, 105683, 105684, 105685, 105686, 105687, 105688, 105689, 105690, 105691 were selected from the ORFs having the nucleotide sequence represented by SEQ ID NO: 77394.

The probes having the nucleotide sequence represented by SEQ ID NOS: 105692, 105693, 105694, 105695, 105696, 105697, 105698, 105699, 105700, 105701, 105702 were selected from the ORFs having the nucleotide sequence represented by SEQ ID NO: 77395.

The probes having the nucleotide sequence represented by SEQ ID NOS: 105703, 105704, 105705, 105706, 105707, 105708, 105709, 105710, 105711, 105712, 105713 were selected from the ORFs having the nucleotide sequence represented by SEQ ID NO: 77396.

The probes having the nucleotide sequence represented by SEQ ID NOS: 105714, 105715, 105716, 105717, 105718, 105719, 105720, 105721, 105722, 105723, 105724 were selected from the ORFs having the nucleotide sequence represented by SEQ ID NO: 77397.

The probes having the nucleotide sequence represented by SEQ ID NOS: 105725, 105726, 105727, 105728, 105729, 105730, 105731, 105732, 105733, 105734, 105735 were selected from the ORFs having the nucleotide sequence represented by SEQ ID NO: 77398.

The probes having the nucleotide sequence represented by SEQ ID NOS: 105736, 105737, 105738, 105739, 105740, 105741, 105742, 105743, 105744, 105745, 105746 were selected from the ORFs having the nucleotide sequence represented by SEQ ID NO: 77399.

The probes having the nucleotide sequence represented by SEQ ID NOS: 105747, 105748, 105749, 105750, 105751, 105752, 105753, 105754, 105755, 105756, 105757 were selected from the ORFs having the nucleotide sequence represented by SEQ ID NO: 77400.

The probes having the nucleotide sequence represented by SEQ ID NOS: 105758, 105759, 105760, 105761, 105762, 105763, 105764, 105765, 105766, 105767, 105768 were selected from the ORFs having the nucleotide sequence represented by SEQ ID NO: 77401.

The probes having the nucleotide sequence represented by SEQ ID NOS: 105769, 105770, 105771, 105772, 105773, 105774, 105775, 105776, 105777, 105778, 105779 were selected from the ORFs having the nucleotide sequence represented by SEQ ID NO: 77402.

The probes having the nucleotide sequence represented by SEQ ID NOS: 105780, 105781, 105782, 105783, 105784, 105785, 105786, 105787, 105788, 105789, 105790 were selected from the ORFs having the nucleotide sequence represented by SEQ ID NO: 77403.

The probes having the nucleotide sequence represented by SEQ ID NOS: 105791, 105792, 105793, 105794, 105795, 105796, 105797, 105798, 105799, 105800, 105801 were selected from the ORFs having the nucleotide sequence represented by SEQ ID NO: 77404.

The probes having the nucleotide sequence represented by SEQ ID NOS: 105802, 105803, 105804, 105805, 105806, 105807, 105808, 105809, 105810, 105811, 105812 were selected from the ORFs having the nucleotide sequence represented by SEQ ID NO: 77405.

The probes having the nucleotide sequence represented by SEQ ID NOS: 105813, 105814, 105815, 105816, 105817, 105818, 105819, 105820, 105821, 105822, 105823 were selected from the ORFs having the nucleotide sequence represented by SEQ ID NO: 77406.

The probes having the nucleotide sequence represented by SEQ ID NOS: 105824, 105825, 105826, 105827, 105828, 105829, 105830, 105831, 105832, 105833, 105834 were selected from the ORFs having the nucleotide sequence represented by SEQ ID NO: 77407.

The probes having the nucleotide sequence represented by SEQ ID NOS: 105835, 105836, 105837, 105838, 105839, 105840, 105841, 105842, 105843, 105844, 105845 were selected from the ORFs having the nucleotide sequence represented by SEQ ID NO: 77408.

The probes having the nucleotide sequence represented by SEQ ID NOS: 105846, 105847, 105848, 105849, 105850, 105851, 105852, 105853, 105854, 105855, 105856 were selected from the ORFs having the nucleotide sequence represented by SEQ ID NO: 77409.

The probes having the nucleotide sequence represented by SEQ ID NOS: 105857, 105858, 105859, 105860, 105861, 105862, 105863, 105864, 105865, 105866, 105867 were selected from the ORFs having the nucleotide sequence represented by SEQ ID NO: 77410.

The probes having the nucleotide sequence represented by SEQ ID NOS: 105868, 105869, 105870, 105871, 105872, 105873, 105874, 105875, 105876, 105877, 105878 were selected from the ORFs having the nucleotide sequence represented by SEQ ID NO: 77411.

The probes having the nucleotide sequence represented by SEQ ID NOS: 105879, 105880, 105881, 105882, 105883, 105884, 105885, 105886, 105887, 105888, 105889 were selected from the ORFs having the nucleotide sequence represented by SEQ ID NO: 77412.

The probes having the nucleotide sequence represented by SEQ ID NOS: 105890, 105891, 105892, 105893, 105894, 105895, 105896, 105897, 105898, 105899, 105900 were selected from the ORFs having the nucleotide sequence represented by SEQ ID NO: 77413.

The probes having the nucleotide sequence represented by SEQ ID NOS: 105901, 105902, 105903, 105904, 105905, 105906, 105907, 105908, 105909, 105910, 105911 were selected from the ORFs having the nucleotide sequence represented by SEQ ID NO: 77414.

The probes having the nucleotide sequence represented by SEQ ID NOS: 105912, 105913, 105914, 105915, 105916, 105917, 105918, 105919, 105920, 105921, 105922 were selected from the ORFs having the nucleotide sequence represented by SEQ ID NO: 77415.

The probes having the nucleotide sequence represented by SEQ ID NOS: 105923, 105924, 105925, 105926, 105927, 105928, 105929, 105930, 105931, 105932, 105933 were selected from the ORFs having the nucleotide sequence represented by SEQ ID NO: 77416.

The probes having the nucleotide sequence represented by SEQ ID NOS: 105934, 105935, 105936, 105937, 105938, 105939, 105940, 105941, 105942, 105943, 105944 were selected from the ORFs having the nucleotide sequence represented by SEQ ID NO: 77417.

The probes having the nucleotide sequence represented by SEQ ID NOS: 105945, 105946, 105947, 105948, 105949, 105950, 105951, 105952, 105953, 105954, 105955 were selected from the ORFs having the nucleotide sequence represented by SEQ ID NO: 77418.

The probes having the nucleotide sequence represented by SEQ ID NOS: 105956, 105957, 105958, 105959, 105960, 105961, 105962, 105963, 105964, 105965, 105966 were selected from the ORFs having the nucleotide sequence represented by SEQ ID NO: 77419.

The probes having the nucleotide sequence represented by SEQ ID NOS: 105967, 105968, 105969, 105970, 105971, 105972, 105973, 105974, 105975, 105976, 105977 were selected from the ORFs having the nucleotide sequence represented by SEQ ID NO: 77420.

The probes having the nucleotide sequence represented by SEQ ID NOS: 105978, 105979, 105980, 105981, 105982, 105983, 105984, 105985, 105986, 105987, 105988 were selected from the ORFs having the nucleotide sequence represented by SEQ ID NO: 77421.

The probes having the nucleotide sequence represented by SEQ ID NOS: 105989, 105990, 105991, 105992, 105993, 105994, 105995, 105996, 105997, 105998, 105999 were selected from the ORFs having the nucleotide sequence represented by SEQ ID NO: 77422.

The probes having the nucleotide sequence represented by SEQ ID NOS: 106000, 106001, 106002, 106003, 106004, 106005, 106006, 106007, 106008, 106009, 106010 were selected from the ORFs having the nucleotide sequence represented by SEQ ID NO: 77423.

The probes having the nucleotide sequence represented by SEQ ID NOS: 106011, 106012, 106013, 106014, 106015, 106016, 106017, 106018, 106019, 106020, 106021 were selected from the ORFs having the nucleotide sequence represented by SEQ ID NO: 77424.

The probes having the nucleotide sequence represented by SEQ ID NOS: 106022, 106023, 106024, 106025, 106026, 106027, 106028, 106029, 106030, 106031, 106032 were selected from the ORFs having the nucleotide sequence represented by SEQ ID NO: 77425.

The probes having the nucleotide sequence represented by SEQ ID NOS: 106033, 106034, 106035, 106036, 106037, 106038, 106039, 106040, 106041, 106042, 106043 were selected from the ORFs having the nucleotide sequence represented by SEQ ID NO: 77426.

The probes having the nucleotide sequence represented by SEQ ID NOS: 106044, 106045, 106046, 106047, 106048, 106049, 106050, 106051, 106052, 106053, 106054 were selected from the ORFs having the nucleotide sequence represented by SEQ ID NO: 77427.

The probes having the nucleotide sequence represented by SEQ ID NOS: 106055, 106056, 106057, 106058, 106059, 106060, 106061, 106062, 106063, 106064, 106065 were selected from the ORFs having the nucleotide sequence represented by SEQ ID NO: 77428.

The probes having the nucleotide sequence represented by SEQ ID NOS: 106066, 106067, 106068, 106069, 106070, 106071, 106072, 106073, 106074, 106075, 106076 were selected from the ORFs having the nucleotide sequence represented by SEQ ID NO: 77429.

The probes having the nucleotide sequence represented by SEQ ID NOS: 106077, 106078, 106079, 106080, 106081, 106082, 106083, 106084, 106085, 106086, 106087 were selected from the ORFs having the nucleotide sequence represented by SEQ ID NO: 77430.

The probes having the nucleotide sequence represented by SEQ ID NOS: 106088, 106089, 106090, 106091, 106092, 106093, 106094, 106095, 106096, 106097, 106098 were selected from the ORFs having the nucleotide sequence represented by SEQ ID NO: 77431.

The probes having the nucleotide sequence represented by SEQ ID NOS: 106099, 106100, 106101, 106102, 106103, 106104, 106105, 106106, 106107, 106108, 106109 were selected from the ORFs having the nucleotide sequence represented by SEQ ID NO: 77432.

The probes having the nucleotide sequence represented by SEQ ID NOS: 106110, 106111, 106112, 106113, 106114, 106115, 106116, 106117, 106118, 106119, 106120 were selected from the ORFs having the nucleotide sequence represented by SEQ ID NO: 77433.

The probes having the nucleotide sequence represented by SEQ ID NOS: 106121, 106122, 106123, 106124, 106125, 106126, 106127, 106128, 106129, 106130, 106131 were selected from the ORFs having the nucleotide sequence represented by SEQ ID NO: 77434.

The probes having the nucleotide sequence represented by SEQ ID NOS: 106132, 106133, 106134, 106135, 106136, 106137, 106138, 106139, 106140, 106141, 106142 were selected from the ORFs having the nucleotide sequence represented by SEQ ID NO: 77435.

The probes having the nucleotide sequence represented by SEQ ID NOS: 106143, 106144, 106145, 106146, 106147, 106148, 106149, 106150, 106151, 106152, 106153 were selected from the ORFs having the nucleotide sequence represented by SEQ ID NO: 77436.

The probes having the nucleotide sequence represented by SEQ ID NOS: 106154, 106155, 106156, 106157, 106158, 106159, 106160, 106161, 106162, 106163, 106164 were selected from the ORFs having the nucleotide sequence represented by SEQ ID NO: 77437.

The probes having the nucleotide sequence represented by SEQ ID NOS: 106165, 106166, 106167, 106168, 106169, 106170, 106171, 106172, 106173, 106174, 106175 were selected from the ORFs having the nucleotide sequence represented by SEQ ID NO: 77438.

The probes having the nucleotide sequence represented by SEQ ID NOS: 106176, 106177, 106178, 106179, 106180, 106181, 106182, 106183, 106184, 106185, 106186 were selected from the ORFs having the nucleotide sequence represented by SEQ ID NO: 77439.

The probes having the nucleotide sequence represented by SEQ ID NOS: 106187, 106188, 106189, 106190, 106191, 106192, 106193, 106194, 106195, 106196, 106197 were selected from the ORFs having the nucleotide sequence represented by SEQ ID NO: 77440.

The probes having the nucleotide sequence represented by SEQ ID NOS: 106198, 106199, 106200, 106201, 106202, 106203, 106204, 106205, 106206, 106207, 106208 were selected from the ORFs having the nucleotide sequence represented by SEQ ID NO: 77441.

The probes having the nucleotide sequence represented by SEQ ID NOS: 106209, 106210, 106211, 106212, 106213, 106214, 106215, 106216, 106217, 106218, 106219 were selected from the ORFs having the nucleotide sequence represented by SEQ ID NO: 77442.

The probes having the nucleotide sequence represented by SEQ ID NOS: 106220, 106221, 106222, 106223, 106224, 106225, 106226, 106227, 106228, 106229, 106230 were selected from the ORFs having the nucleotide sequence represented by SEQ ID NO: 77443.

The probes having the nucleotide sequence represented by SEQ ID NOS: 106231, 106232, 106233, 106234, 106235, 106236, 106237, 106238, 106239, 106240, 106241 were selected from the ORFs having the nucleotide sequence represented by SEQ ID NO: 77444.

The probes having the nucleotide sequence represented by SEQ ID NOS: 106242, 106243, 106244, 106245, 106246, 106247, 106248, 106249, 106250, 106251, 106252 were selected from the ORFs having the nucleotide sequence represented by SEQ ID NO: 77445.

The probes having the nucleotide sequence represented by SEQ ID NOS: 106253, 106254, 106255, 106256, 106257, 106258, 106259, 106260, 106261, 106262, 106263 were selected from the ORFs having the nucleotide sequence represented by SEQ ID NO: 77446.

The probes having the nucleotide sequence represented by SEQ ID NOS: 106264, 106265, 106266, 106267, 106268, 106269, 106270, 106271, 106272, 106273, 106274 were selected from the ORFs having the nucleotide sequence represented by SEQ ID NO: 77447.

The probes having the nucleotide sequence represented by SEQ ID NOS: 106275, 106276, 106277, 106278, 106279, 106280, 106281, 106282, 106283, 106284, 106285 were selected from the ORFs having the nucleotide sequence represented by SEQ ID NO: 77448.

The probes having the nucleotide sequence represented by SEQ ID NOS: 106286, 106287, 106288, 106289, 106290, 106291, 106292, 106293, 106294, 106295, 106296 were selected from the ORFs having the nucleotide sequence represented by SEQ ID NO: 77449.

The probes having the nucleotide sequence represented by SEQ ID NOS: 106297, 106298, 106299, 106300, 106301, 106302, 106303, 106304, 106305, 106306, 106307 were selected from the ORFs having the nucleotide sequence represented by SEQ ID NO: 77450.

The probes having the nucleotide sequence represented by SEQ ID NOS: 106308, 106309, 106310, 106311, 106312, 106313, 106314, 106315, 106316, 106317, 106318 were selected from the ORFs having the nucleotide sequence represented by SEQ ID NO: 77451.

The probes having the nucleotide sequence represented by SEQ ID NOS: 106319, 106320, 106321, 106322, 106323, 106324, 106325, 106326, 106327, 106328, 106329 were selected from the ORFs having the nucleotide sequence represented by SEQ ID NO: 77452.

The probes having the nucleotide sequence represented by SEQ ID NOS: 106330, 106331, 106332, 106333, 106334, 106335, 106336, 106337, 106338, 106339, 106340 were selected from the ORFs having the nucleotide sequence represented by SEQ ID NO: 77453.

The probes having the nucleotide sequence represented by SEQ ID NOS: 106341, 106342, 106343, 106344, 106345, 106346, 106347, 106348, 106349, 106350, 106351 were selected from the ORFs having the nucleotide sequence represented by SEQ ID NO: 77454.

The probes having the nucleotide sequence represented by SEQ ID NOS: 106352, 106353, 106354, 106355, 106356, 106357, 106358, 106359, 106360, 106361, 106362 were selected from the ORFs having the nucleotide sequence represented by SEQ ID NO: 77455.

The probes having the nucleotide sequence represented by SEQ ID NOS: 106363, 106364, 106365, 106366, 106367, 106368, 106369, 106370, 106371, 106372, 106373 were selected from the ORFs having the nucleotide sequence represented by SEQ ID NO: 77456.

The probes having the nucleotide sequence represented by SEQ ID NOS: 106374, 106375, 106376, 106377, 106378, 106379, 106380, 106381, 106382, 106383, 106384 were selected from the ORFs having the nucleotide sequence represented by SEQ ID NO: 77457.

The probes having the nucleotide sequence represented by SEQ ID NOS: 106385, 106386, 106387, 106388, 106389, 106390, 106391, 106392, 106393, 106394, 106395 were selected from the ORFs having the nucleotide sequence represented by SEQ ID NO: 77458.

The probes having the nucleotide sequence represented by SEQ ID NOS: 106396, 106397, 106398, 106399, 106400, 106401, 106402, 106403, 106404, 106405, 106406 were selected from the ORFs having the nucleotide sequence represented by SEQ ID NO: 77459.

The probes having the nucleotide sequence represented by SEQ ID NOS: 106407, 106408, 106409, 106410, 106411, 106412, 106413, 106414, 106415, 106416, 106417, 106418, 106419, 106420, 106421, 106422, 106423, 106424, 106425, 106426, 106427, 106428 were selected from the ORFs having the nucleotide sequence represented by SEQ ID NO: 77460.

The probes having the nucleotide sequence represented by SEQ ID NOS: 106429, 106430, 106431, 106432, 106433, 106434, 106435, 106436, 106437, 106438, 106439 were selected from the ORFs having the nucleotide sequence represented by SEQ ID NO: 77461.

The probes having the nucleotide sequence represented by SEQ ID NOS: 106440, 106441, 106442, 106443, 106444, 106445, 106446, 106447, 106448, 106449, 106450 were selected from the ORFs having the nucleotide sequence represented by SEQ ID NO: 77462.

The probes having the nucleotide sequence represented by SEQ ID NOS: 106451, 106452, 106453, 106454, 106455, 106456, 106457, 106458, 106459, 106460, 106461 were selected from the ORFs having the nucleotide sequence represented by SEQ ID NO: 77463.

The probes having the nucleotide sequence represented by SEQ ID NOS: 106462, 106463, 106464, 106465, 106466, 106467, 106468, 106469, 106470, 106471, 106472 were selected from the ORFs having the nucleotide sequence represented by SEQ ID NO: 77464.

The probes having the nucleotide sequence represented by SEQ ID NOS: 106473, 106474, 106475, 106476, 106477, 106478, 106479, 106480, 106481, 106482, 106483 were selected from the ORFs having the nucleotide sequence represented by SEQ ID NO: 77465.

The probes having the nucleotide sequence represented by SEQ ID NOS: 106484, 106485, 106486, 106487, 106488, 106489, 106490, 106491, 106492, 106493, 106494 were selected from the ORFs having the nucleotide sequence represented by SEQ ID NO: 77466.

The probes having the nucleotide sequence represented by SEQ ID NOS: 106495, 106496, 106497, 106498, 106499, 106500, 106501, 106502, 106503, 106504, 106505 were selected from the ORFs having the nucleotide sequence represented by SEQ ID NO: 77467.

The probes having the nucleotide sequence represented by SEQ ID NOS: 106506, 106507, 106508, 106509, 106510, 106511, 106512, 106513, 106514, 106515, 106516 were selected from the ORFs having the nucleotide sequence represented by SEQ ID NO: 77468.

The probes having the nucleotide sequence represented by SEQ ID NOS: 106517, 106518, 106519, 106520, 106521, 106522, 106523, 106524, 106525, 106526, 106527 were selected from the ORFs having the nucleotide sequence represented by SEQ ID NO: 77469.

The probes having the nucleotide sequence represented by SEQ ID NOS: 106528, 106529, 106530, 106531, 106532, 106533, 106534, 106535, 106536, 106537, 106538 were selected from the ORFs having the nucleotide sequence represented by SEQ ID NO: 77470.

The probes having the nucleotide sequence represented by SEQ ID NOS: 106539, 106540, 106541, 106542, 106543, 106544, 106545, 106546, 106547, 106548, 106549 were selected from the ORFs having the nucleotide sequence represented by SEQ ID NO: 77471.

The probes having the nucleotide sequence represented by SEQ ID NOS: 106550, 106551, 106552, 106553, 106554, 106555, 106556, 106557, 106558, 106559, 106560 were selected from the ORFs having the nucleotide sequence represented by SEQ ID NO: 77472.

The probes having the nucleotide sequence represented by SEQ ID NOS: 106561, 106562, 106563, 106564, 106565, 106566, 106567, 106568, 106569, 106570, 106571 were selected from the ORFs having the nucleotide sequence represented by SEQ ID NO: 77473.

The probes having the nucleotide sequence represented by SEQ ID NOS: 106572, 106573, 106574, 106575, 106576, 106577, 106578, 106579, 106580, 106581, 106582 were selected from the ORFs having the nucleotide sequence represented by SEQ ID NO: 77474.

The probes having the nucleotide sequence represented by SEQ ID NOS: 106583, 106584, 106585, 106586, 106587, 106588, 106589, 106590, 106591, 106592, 106593 were selected from the ORFs having the nucleotide sequence represented by SEQ ID NO: 77475.

The probes having the nucleotide sequence represented by SEQ ID NOS: 106594, 106595, 106596, 106597, 106598, 106599, 106600, 106601, 106602, 106603, 106604 were selected from the ORFs having the nucleotide sequence represented by SEQ ID NO: 77476.

The probes having the nucleotide sequence represented by SEQ ID NOS: 106605, 106606, 106607, 106608, 106609, 106610, 106611, 106612, 106613, 106614, 106615 were selected from the ORFs having the nucleotide sequence represented by SEQ ID NO: 77477.

The probes having the nucleotide sequence represented by SEQ ID NOS: 106616, 106617, 106618, 106619, 106620, 106621, 106622, 106623, 106624, 106625, 106626 were selected from the ORFs having the nucleotide sequence represented by SEQ ID NO: 77478.

The probes having the nucleotide sequence represented by SEQ ID NOS: 106627, 106628, 106629, 106630, 106631, 106632, 106633, 106634, 106635, 106636, 106637 were selected from the ORFs having the nucleotide sequence represented by SEQ ID NO: 77479.

The probes having the nucleotide sequence represented by SEQ ID NOS: 106638, 106639, 106640, 106641, 106642, 106643, 106644, 106645, 106646, 106647, 106648 were selected from the ORFs having the nucleotide sequence represented by SEQ ID NO: 77480.

The probes having the nucleotide sequence represented by SEQ ID NOS: 106649, 106650, 106651, 106652, 106653, 106654, 106655, 106656, 106657, 106658, 106659 were selected from the ORFs having the nucleotide sequence represented by SEQ ID NO: 77481.

The probes having the nucleotide sequence represented by SEQ ID NOS: 106660, 106661, 106662, 106663, 106664, 106665, 106666, 106667, 106668, 106669, 106670 were selected from the ORFs having the nucleotide sequence represented by SEQ ID NO: 77482.

The probes having the nucleotide sequence represented by SEQ ID NOS: 106671, 106672, 106673, 106674, 106675, 106676, 106677, 106678, 106679, 106680, 106681 were selected from the ORFs having the nucleotide sequence represented by SEQ ID NO: 77483.

The probes having the nucleotide sequence represented by SEQ ID NOS: 106682, 106683, 106684, 106685, 106686, 106687, 106688, 106689, 106690, 106691, 106692 were selected from the ORFs having the nucleotide sequence represented by SEQ ID NO: 77484.

The probes having the nucleotide sequence represented by SEQ ID NOS: 106693, 106694, 106695, 106696, 106697, 106698, 106699, 106700, 106701, 106702, 106703 were selected from the ORFs having the nucleotide sequence represented by SEQ ID NO: 77485.

The probes having the nucleotide sequence represented by SEQ ID NOS: 106704, 106705, 106706, 106707, 106708, 106709, 106710, 106711, 106712, 106713, 106714 were selected from the ORFs having the nucleotide sequence represented by SEQ ID NO: 77486.

The probes having the nucleotide sequence represented by SEQ ID NOS: 106715, 106716, 106717, 106718, 106719, 106720, 106721, 106722, 106723, 106724, 106725 were selected from the ORFs having the nucleotide sequence represented by SEQ ID NO: 77487.

The probes having the nucleotide sequence represented by SEQ ID NOS: 106726, 106727, 106728, 106729, 106730, 106731, 106732, 106733, 106734, 106735, 106736 were selected from the ORFs having the nucleotide sequence represented by SEQ ID NO: 77488.

The probes having the nucleotide sequence represented by SEQ ID NOS: 106737, 106738, 106739, 106740, 106741, 106742, 106743, 106744, 106745, 106746, 106747 were selected from the ORFs having the nucleotide sequence represented by SEQ ID NO: 77489.

The probes having the nucleotide sequence represented by SEQ ID NOS: 106748, 106749, 106750, 106751, 106752, 106753, 106754, 106755, 106756, 106757, 106758 were selected from the ORFs having the nucleotide sequence represented by SEQ ID NO: 77490.

The probes having the nucleotide sequence represented by SEQ ID NOS: 106759, 106760, 106761, 106762, 106763, 106764, 106765, 106766, 106767, 106768, 106769 were selected from the ORFs having the nucleotide sequence represented by SEQ ID NO: 77491.

The probes having the nucleotide sequence represented by SEQ ID NOS: 106770, 106771, 106772, 106773, 106774, 106775, 106776, 106777, 106778, 106779, 106780 were selected from the ORFs having the nucleotide sequence represented by SEQ ID NO: 77492.

The probes having the nucleotide sequence represented by SEQ ID NOS: 106781, 106782, 106783, 106784, 106785, 106786, 106787, 106788, 106789, 106790, 106791 were selected from the ORFs having the nucleotide sequence represented by SEQ ID NO: 77493.

The probes having the nucleotide sequence represented by SEQ ID NOS: 106792, 106793, 106794, 106795, 106796, 106797, 106798, 106799, 106800, 106801, 106802 were selected from the ORFs having the nucleotide sequence represented by SEQ ID NO: 77494.

The probes having the nucleotide sequence represented by SEQ ID NOS: 106803, 106804, 106805, 106806, 106807, 106808, 106809, 106810, 106811, 106812, 106813 were selected from the ORFs having the nucleotide sequence represented by SEQ ID NO: 77495.

The probes having the nucleotide sequence represented by SEQ ID NOS: 106814, 106815, 106816, 106817, 106818, 106819, 106820, 106821, 106822, 106823, 106824 were selected from the ORFs having the nucleotide sequence represented by SEQ ID NO: 77496.

The probes having the nucleotide sequence represented by SEQ ID NOS: 106825, 106826, 106827, 106828, 106829, 106830, 106831, 106832, 106833, 106834, 106835 were selected from the ORFs having the nucleotide sequence represented by SEQ ID NO: 77497.

The probes having the nucleotide sequence represented by SEQ ID NOS: 106836, 106837, 106838, 106839, 106840, 106841, 106842, 106843, 106844, 106845, 106846 were selected from the ORFs having the nucleotide sequence represented by SEQ ID NO: 77498.

The probes having the nucleotide sequence represented by SEQ ID NOS: 106847, 106848, 106849, 106850, 106851, 106852, 106853, 106854, 106855, 106856, 106857 were selected from the ORFs having the nucleotide sequence represented by SEQ ID NO: 77499.

The probes having the nucleotide sequence represented by SEQ ID NOS: 106858, 106859, 106860, 106861, 106862, 106863, 106864, 106865, 106866, 106867, 106868 were selected from the ORFs having the nucleotide sequence represented by SEQ ID NO: 77500.

The probes having the nucleotide sequence represented by SEQ ID NOS: 106869, 106870, 106871, 106872, 106873, 106874, 106875, 106876, 106877, 106878, 106879 were selected from the ORFs having the nucleotide sequence represented by SEQ ID NO: 77501.

The probes having the nucleotide sequence represented by SEQ ID NOS: 106880, 106881, 106882, 106883, 106884, 106885, 106886, 106887, 106888, 106889, 106890 were selected from the ORFs having the nucleotide sequence represented by SEQ ID NO: 77502.

The probes having the nucleotide sequence represented by SEQ ID NOS: 106891, 106892, 106893, 106894, 106895, 106896, 106897, 106898, 106899, 106900, 106901 were selected from the ORFs having the nucleotide sequence represented by SEQ ID NO: 77503.

The probes having the nucleotide sequence represented by SEQ ID NOS: 106902, 106903, 106904, 106905, 106906, 106907, 106908, 106909, 106910, 106911, 106912 were selected from the ORFs having the nucleotide sequence represented by SEQ ID NO: 77504.

The probes having the nucleotide sequence represented by SEQ ID NOS: 106913, 106914, 106915, 106916, 106917, 106918, 106919, 106920, 106921, 106922, 106923 were selected from the ORFs having the nucleotide sequence represented by SEQ ID NO: 77505.

The probes having the nucleotide sequence represented by SEQ ID NOS: 106924, 106925, 106926, 106927, 106928, 106929, 106930, 106931, 106932, 106933, 106934 were selected from the ORFs having the nucleotide sequence represented by SEQ ID NO: 77506.

The probes having the nucleotide sequence represented by SEQ ID NOS: 106935, 106936, 106937, 106938, 106939, 106940, 106941, 106942, 106943, 106944, 106945 were selected from the ORFs having the nucleotide sequence represented by SEQ ID NO: 77507.

The probes having the nucleotide sequence represented by SEQ ID NOS: 106946, 106947, 106948, 106949, 106950, 106951, 106952, 106953, 106954, 106955, 106956 were selected from the ORFs having the nucleotide sequence represented by SEQ ID NO: 77508.

The probes having the nucleotide sequence represented by SEQ ID NOS: 106957, 106958, 106959, 106960, 106961, 106962, 106963, 106964, 106965, 106966, 106967 were selected from the ORFs having the nucleotide sequence represented by SEQ ID NO: 77509.

The probes having the nucleotide sequence represented by SEQ ID NOS: 106968, 106969, 106970, 106971, 106972, 106973, 106974, 106975, 106976, 106977, 106978 were selected from the ORFs having the nucleotide sequence represented by SEQ ID NO: 77510.

The probes having the nucleotide sequence represented by SEQ ID NOS: 106979, 106980, 106981, 106982, 106983, 106984, 106985, 106986, 106987, 106988, 106989 were selected from the ORFs having the nucleotide sequence represented by SEQ ID NO: 77511.

The probes having the nucleotide sequence represented by SEQ ID NOS: 106990, 106991, 106992, 106993, 106994, 106995, 106996, 106997, 106998, 106999, 107000 were selected from the ORFs having the nucleotide sequence represented by SEQ ID NO: 77512.

The probes having the nucleotide sequence represented by SEQ ID NOS: 107001, 107002, 107003, 107004, 107005, 107006, 107007, 107008, 107009, 107010, 107011 were selected from the ORFs having the nucleotide sequence represented by SEQ ID NO: 77513.

The probes having the nucleotide sequence represented by SEQ ID NOS: 107012, 107013, 107014, 107015, 107016, 107017, 107018, 107019, 107020, 107021, 107022 were selected from the ORFs having the nucleotide sequence represented by SEQ ID NO: 77514.

The probes having the nucleotide sequence represented by SEQ ID NOS: 107023, 107024, 107025, 107026, 107027, 107028, 107029, 107030, 107031, 107032, 107033 were selected from the ORFs having the nucleotide sequence represented by SEQ ID NO: 77515.

The probes having the nucleotide sequence represented by SEQ ID NOS: 107034, 107035, 107036, 107037, 107038, 107039, 107040, 107041, 107042, 107043, 107044 were selected from the ORFs having the nucleotide sequence represented by SEQ ID NO: 77516.

The probes having the nucleotide sequence represented by SEQ ID NOS: 107045, 107046, 107047, 107048, 107049, 107050, 107051, 107052, 107053, 107054, 107055 were selected from the ORFs having the nucleotide sequence represented by SEQ ID NO: 77517.

The probes having the nucleotide sequence represented by SEQ ID NOS: 107056, 107057, 107058, 107059, 107060, 107061, 107062, 107063, 107064, 107065, 107066 were selected from the ORFs having the nucleotide sequence represented by SEQ ID NO: 77518.

The probes having the nucleotide sequence represented by SEQ ID NOS: 107067, 107068, 107069, 107070, 107071, 107072, 107073, 107074, 107075, 107076, 107077 were selected from the ORFs having the nucleotide sequence represented by SEQ ID NO: 77519.

The probes having the nucleotide sequence represented by SEQ ID NOS: 107078, 107079, 107080, 107081, 107082, 107083, 107084, 107085, 107086, 107087, 107088 were selected from the ORFs having the nucleotide sequence represented by SEQ ID NO: 77520.

The probes having the nucleotide sequence represented by SEQ ID NOS: 107089, 107090, 107091, 107092, 107093, 107094, 107095, 107096, 107097, 107098, 107099, 107100, 107101, 107102, 107103, 107104, 107105, 107106, 107107, 107108, 107109, 107110 were selected from the ORFs having the nucleotide sequence represented by SEQ ID NO: 77521.

The probes having the nucleotide sequence represented by SEQ ID NOS: 107111, 107112, 107113, 107114, 107115, 107116, 107117, 107118, 107119, 107120, 107121 were selected from the ORFs having the nucleotide sequence represented by SEQ ID NO: 77522.

The probes having the nucleotide sequence represented by SEQ ID NOS: 107122, 107123, 107124, 107125, 107126, 107127, 107128, 107129, 107130, 107131, 107132 were selected from the ORFs having the nucleotide sequence represented by SEQ ID NO: 77523.

The probes having the nucleotide sequence represented by SEQ ID NOS: 107133, 107134, 107135, 107136, 107137, 107138, 107139, 107140, 107141, 107142, 107143 were selected from the ORFs having the nucleotide sequence represented by SEQ ID NO: 77524.

The probes having the nucleotide sequence represented by SEQ ID NOS: 107144, 107145, 107146, 107147, 107148, 107149, 107150, 107151, 107152, 107153, 107154 were selected from the ORFs having the nucleotide sequence represented by SEQ ID NO: 77525.

The probes having the nucleotide sequence represented by SEQ ID NOS: 107155, 107156, 107157, 107158, 107159, 107160, 107161, 107162, 107163, 107164, 107165 were selected from the ORFs having the nucleotide sequence represented by SEQ ID NO: 77526.

The probes having the nucleotide sequence represented by SEQ ID NOS: 107166, 107167, 107168, 107169, 107170, 107171, 107172, 107173, 107174, 107175, 107176 were selected from the ORFs having the nucleotide sequence represented by SEQ ID NO: 77527.

The probes having the nucleotide sequence represented by SEQ ID NOS: 107177, 107178, 107179, 107180, 107181, 107182, 107183, 107184, 107185, 107186, 107187 were selected from the ORFs having the nucleotide sequence represented by SEQ ID NO: 77528.

The probes having the nucleotide sequence represented by SEQ ID NOS: 107188, 107189, 107190, 107191, 107192, 107193, 107194, 107195, 107196, 107197, 107198 were selected from the ORFs having the nucleotide sequence represented by SEQ ID NO: 77529.

The probes having the nucleotide sequence represented by SEQ ID NOS: 107199, 107200, 107201, 107202, 107203, 107204, 107205, 107206, 107207, 107208, 107209 were selected from the ORFs having the nucleotide sequence represented by SEQ ID NO: 77530.

The probes having the nucleotide sequence represented by SEQ ID NOS: 107210, 107211, 107212, 107213, 107214, 107215, 107216, 107217, 107218, 107219, 107220 were selected from the ORFs having the nucleotide sequence represented by SEQ ID NO: 77531.

The probes having the nucleotide sequence represented by SEQ ID NOS: 107221, 107222, 107223, 107224, 107225, 107226, 107227, 107228, 107229, 107230, 107231 were selected from the ORFs having the nucleotide sequence represented by SEQ ID NO: 77532.

The probes having the nucleotide sequence represented by SEQ ID NOS: 107232, 107233, 107234, 107235, 107236, 107237, 107238, 107239, 107240, 107241, 107242 were selected from the ORFs having the nucleotide sequence represented by SEQ ID NO: 77533.

The probes having the nucleotide sequence represented by SEQ ID NOS: 107243, 107244, 107245, 107246, 107247, 107248, 107249, 107250, 107251, 107252, 107253 were selected from the ORFs having the nucleotide sequence represented by SEQ ID NO: 77534.

The probes having the nucleotide sequence represented by SEQ ID NOS: 107254, 107255, 107256, 107257, 107258, 107259, 107260, 107261, 107262, 107263, 107264, 107265, 107266, 107267, 107268, 107269, 107270, 107271, 107272, 107273, 107274, 107275 were selected from the ORFs having the nucleotide sequence represented by SEQ ID NO: 77535.

The probes having the nucleotide sequence represented by SEQ ID NOS: 107276, 107277, 107278, 107279, 107280, 107281, 107282, 107283, 107284, 107285, 107286 were selected from the ORFs having the nucleotide sequence represented by SEQ ID NO: 77536.

The probes having the nucleotide sequence represented by SEQ ID NOS: 107287, 107288, 107289, 107290, 107291, 107292, 107293, 107294, 107295, 107296, 107297 were selected from the ORFs having the nucleotide sequence represented by SEQ ID NO: 77537.

The probes having the nucleotide sequence represented by SEQ ID NOS: 107298, 107299, 107300, 107301, 107302, 107303, 107304, 107305, 107306, 107307, 107308 were selected from the ORFs having the nucleotide sequence represented by SEQ ID NO: 77538.

The probes having the nucleotide sequence represented by SEQ ID NOS: 107309, 107310, 107311, 107312, 107313, 107314, 107315, 107316, 107317, 107318, 107319 were selected from the ORFs having the nucleotide sequence represented by SEQ ID NO: 77539.

The probes having the nucleotide sequence represented by SEQ ID NOS: 107320, 107321, 107322, 107323, 107324, 107325, 107326, 107327, 107328, 107329, 107330 were selected from the ORFs having the nucleotide sequence represented by SEQ ID NO: 77540.

The probes having the nucleotide sequence represented by SEQ ID NOS: 107331, 107332, 107333, 107334, 107335, 107336, 107337, 107338, 107339, 107340, 107341 were selected from the ORFs having the nucleotide sequence represented by SEQ ID NO: 77541.

The probes having the nucleotide sequence represented by SEQ ID NOS: 107342, 107343, 107344, 107345, 107346, 107347, 107348, 107349, 107350, 107351, 107352 were selected from the ORFs having the nucleotide sequence represented by SEQ ID NO: 77542.

The probes having the nucleotide sequence represented by SEQ ID NOS: 107353, 107354, 107355, 107356, 107357, 107358, 107359, 107360, 107361, 107362, 107363 were selected from the ORFs having the nucleotide sequence represented by SEQ ID NO: 77543.

The probes having the nucleotide sequence represented by SEQ ID NOS: 107364, 107365, 107366, 107367, 107368, 107369, 107370, 107371, 107372, 107373, 107374 were selected from the ORFs having the nucleotide sequence represented by SEQ ID NO: 77544.

The probes having the nucleotide sequence represented by SEQ ID NOS: 107375, 107376, 107377, 107378, 107379, 107380, 107381, 107382, 107383, 107384, 107385 were selected from the ORFs having the nucleotide sequence represented by SEQ ID NO: 77545.

The probes having the nucleotide sequence represented by SEQ ID NOS: 107386, 107387, 107388, 107389, 107390, 107391, 107392, 107393, 107394, 107395, 107396 were selected from the ORFs having the nucleotide sequence represented by SEQ ID NO: 77546.

The probes having the nucleotide sequence represented by SEQ ID NOS: 107397, 107398, 107399, 107400, 107401, 107402, 107403, 107404, 107405, 107406, 107407 were selected from the ORFs having the nucleotide sequence represented by SEQ ID NO: 77547.

The probes having the nucleotide sequence represented by SEQ ID NOS: 107408, 107409, 107410, 107411, 107412, 107413, 107414, 107415, 107416, 107417, 107418 were selected from the ORFs having the nucleotide sequence represented by SEQ ID NO: 77548.

The probes having the nucleotide sequence represented by SEQ ID NOS: 107419, 107420, 107421, 107422, 107423, 107424, 107425, 107426, 107427, 107428, 107429, 107430, 107431, 107432, 107433, 107434, 107435, 107436, 107437, 107438, 107439, 107440 were selected from the ORFs having the nucleotide sequence represented by SEQ ID NO: 77549.

The probes having the nucleotide sequence represented by SEQ ID NOS: 107441, 107442, 107443, 107444, 107445, 107446, 107447, 107448, 107449, 107450, 107451 were selected from the ORFs having the nucleotide sequence represented by SEQ ID NO: 77550.

The probes having the nucleotide sequence represented by SEQ ID NOS: 107452, 107453, 107454, 107455, 101456, 107457, 107458, 107459, 107460, 107461, 107462 were selected from the ORFs having the nucleotide sequence represented by SEQ ID NO: 77551.

The probes having the nucleotide sequence represented by SEQ ID NOS: 107463, 107464, 107465, 107466, 107467, 107468, 107469, 107470, 107471, 107472, 107473 were selected from the ORFs having the nucleotide sequence represented by SEQ ID NO: 77552.

The probes having the nucleotide sequence represented by SEQ ID NOS: 107474, 107475, 107476, 107477, 107478, 107479, 107480, 107481, 107482, 107483, 10748.4, 107485, 107486, 107487, 107488, 107489, 107490, 107491, 107492, 107493, 107494, 107495 were selected from the ORFs having the nucleotide sequence represented by SEQ ID NO: 77553.

The probes having the nucleotide sequence represented by SEQ ID NOS: 107496, 107497, 107498, 107499, 107500, 107501, 107502, 107503, 107504, 107505, 107506 were selected from the ORFs having the nucleotide sequence represented by SEQ ID NO: 77554.

The probes having the nucleotide sequence represented by SEQ ID NOS: 107507, 107508, 107509, 107510, 107511, 107512, 107513, 107514, 107515, 107516, 107517 were selected from the ORFs having the nucleotide sequence represented by SEQ ID NO: 77555.

The probes having the nucleotide sequence represented by SEQ ID NOS: 107518, 107519, 107520, 107521, 107522, 107523, 107524, 107525, 107526, 107527, 107528 were selected from the ORFs having the nucleotide sequence represented by SEQ ID NO: 77556.

The probes having the nucleotide sequence represented by SEQ ID NOS: 107529, 107530, 107531, 107532, 107533, 107534, 107535, 107536, 107537, 107538, 107539 were selected from the ORFs having the nucleotide sequence represented by SEQ ID NO: 77557.

The probes having the nucleotide sequence represented by SEQ ID NOS: 107540, 107541, 107542, 107543, 107544, 107545, 107546, 107547, 107548, 107549, 107550 were selected from the ORFs having the nucleotide sequence represented by SEQ ID NO: 77558.

The probes having the nucleotide sequence represented by SEQ ID NOS: 107551, 107552, 107553, 107554, 107555, 107556, 107557, 107558, 107559, 107560, 107561 were selected from the ORFs having the nucleotide sequence represented by SEQ ID NO: 77559.

The probes having the nucleotide sequence represented by SEQ ID NOS: 107562, 107563, 107564, 107565, 107566, 107567, 107568, 107569, 107570, 107571, 107572 were selected from the ORFs having the nucleotide sequence represented by SEQ ID NO: 77560.

The probes having the nucleotide sequence represented by SEQ ID NOS: 107573, 107574, 107575, 107576, 107577, 107578, 107579, 107580, 107581, 107582, 107583 were selected from the ORFs having the nucleotide sequence represented by SEQ ID NO: 77561.

The probes having the nucleotide sequence represented by SEQ ID NOS: 107584, 107585, 107586, 107587, 107588, 107589, 107590, 107591, 107592, 107593, 107594 were selected from the ORFs having the nucleotide sequence represented by SEQ ID NO: 77562.

The probes having the nucleotide sequence represented by SEQ ID NOS: 107595, 107596, 107597, 107598, 107599, 107600, 107601, 107602, 107603, 107604, 107605 were selected from the ORFs having the nucleotide sequence represented by SEQ ID NO: 77563.

The probes having the nucleotide sequence represented by SEQ ID NOS: 107606, 107607, 107608, 107609, 107610, 107611, 107612, 107613, 107614, 107615, 107616 were selected from the ORFs having the nucleotide sequence represented by SEQ ID NO: 77564.

The probes having the nucleotide sequence represented by SEQ ID NOS: 107617, 107618, 107619, 107620, 107621, 107622, 107623, 107624, 107625, 107626, 107627 were selected from the ORFs having the nucleotide sequence represented by SEQ ID NO: 77565.

The probes having the nucleotide sequence represented by SEQ ID NOS: 107628, 107629, 107630, 107631, 107632, 107633, 107634, 107635, 107636, 107637, 107638 were selected from the ORFs having the nucleotide sequence represented by SEQ ID NO: 77566.

The probes having the nucleotide sequence represented by SEQ ID NOS: 107639, 107640, 107641, 107642, 107643, 107644, 107645, 107646, 107647, 107648, 107649 were selected from the ORFs having the nucleotide sequence represented by SEQ ID NO: 77567.

The probes having the nucleotide sequence represented by SEQ ID NOS: 107650, 107651, 107652, 107653, 107654, 107655, 107656, 107657, 107658, 107659, 107660 were selected from the ORFs having the nucleotide sequence represented by SEQ ID NO: 77568.

The probes having the nucleotide sequence represented by SEQ ID NOS: 107661, 107662, 107663, 107664, 107665, 107666, 107667, 107668, 107669, 107670, 107671 were selected from the ORFs having the nucleotide sequence represented by SEQ ID NO: 77569.

The probes having the nucleotide sequence represented by SEQ ID NOS: 107672, 107673, 107674, 107675, 107676, 107677, 107678, 107679, 107680, 107681, 107682 were selected from the ORFs having the nucleotide sequence represented by SEQ ID NO: 77570.

The probes having the nucleotide sequence represented by SEQ ID NOS: 107683, 107684, 107685, 107686, 107687, 107688, 107689, 107690, 107691, 107692, 107693 were selected from the ORFs having the nucleotide sequence represented by SEQ ID NO: 77571.

The probes having the nucleotide sequence represented by SEQ ID NOS: 107694, 107695, 107696, 107697, 107698, 107699, 107700, 107701, 107702, 107703, 107704 were selected from the ORFs having the nucleotide sequence represented by SEQ ID NO: 77572.

The probes having the nucleotide sequence represented by SEQ ID NOS: 107705, 107706, 107707, 107708, 107709, 107710, 107711, 107712, 107713, 107714, 107715 were selected from the ORFs having the nucleotide sequence represented by SEQ ID NO: 77573.

The probes having the nucleotide sequence represented by SEQ ID NOS: 107716, 107717, 107718, 107719, 107720, 107721, 107722, 107723, 107724, 107725, 107726 were selected from the ORFs having the nucleotide sequence represented by SEQ ID NO: 77574.

The probes having the nucleotide sequence represented by SEQ ID NOS: 107727, 107728, 107729, 107730, 107731, 107732, 107733, 107734, 107735, 107736, 107737 were selected from the ORFs having the nucleotide sequence represented by SEQ ID NO: 77575.

The probes having the nucleotide sequence represented by SEQ. ID NOS: 107738, 107739, 107740, 107741, 107742, 107743, 107744, 107745, 107746, 107747, 107748 were selected from the ORFs having the nucleotide sequence represented by SEQ ID NO: 77576.

The probes having the nucleotide sequence represented by SEQ ID NOS: 107749, 107750, 107751, 107752, 107753, 107754, 107755, 107756, 107757, 107758, 107759 were selected from the ORFs having the nucleotide sequence represented by SEQ ID NO: 77577.

The probes having the nucleotide sequence represented by SEQ ID NOS: 107760, 107761, 107762, 107763, 107764, 107765, 107766, 107767, 107768, 107769, 107770 were selected from the ORFs having the nucleotide sequence represented by SEQ ID NO: 77578.

The probes having the nucleotide sequence represented by SEQ ID NOS: 107771, 107772, 107773, 107774, 107775, 107776, 107777, 107778, 107779, 107780, 107781 were selected from the ORFs having the nucleotide sequence represented by SEQ ID NO: 77579.

The probes having the nucleotide sequence represented by SEQ ID NOS: 107782, 107783, 107784, 107785, 107786, 107787, 107788, 107789, 107790, 107791, 107792 were selected from the ORFs having the nucleotide sequence represented by SEQ ID NO: 77580.

The probes having the nucleotide sequence represented by SEQ ID NOS: 107793, 107794, 107795, 107796, 107797, 107798, 107799, 107800, 107801, 107802, 107803 were selected from the ORFs having the nucleotide sequence represented by SEQ ID NO: 77581.

The probes having the nucleotide sequence represented by SEQ ID NOS: 107804, 107805, 107806, 107807, 107808, 107809, 107810, 107811, 107812, 107813, 107814 were selected from the ORFs having the nucleotide sequence represented by SEQ ID NO: 77582.

The probes having the nucleotide sequence represented by SEQ ID NOS: 107815, 107816, 107817, 107818, 107819, 107820, 107821, 107822, 107823, 107824, 107825 were selected from the ORFs having the nucleotide sequence represented by SEQ ID NO: 77583.

The probes having the nucleotide sequence represented by SEQ ID NOS: 107826, 107827, 107828, 107829, 107830, 107831, 107832, 107833, 107834, 107835, 107836 were selected from the ORFs having the nucleotide sequence represented by SEQ ID NO: 77584.

The probes having the nucleotide sequence represented by SEQ ID NOS: 107837, 107838, 107839, 107840, 107841, 107842, 107843, 107844, 107845, 107846, 107847 were selected from the ORFs having the nucleotide sequence represented by SEQ ID NO: 77585.

The probes having the nucleotide sequence represented by SEQ ID NOS: 107848, 107849, 107850, 107851, 107852, 107853, 107854, 107855, 107856, 107857, 107858 were selected from the ORFs having the nucleotide sequence represented by SEQ ID NO: 77586.

The probes having the nucleotide sequence represented by SEQ ID NOS: 107859, 107860, 107861, 107862, 107863, 107864, 107865, 107866, 107867, 107868, 107869 were selected from the ORFs having the nucleotide sequence represented by SEQ ID NO: 77587.

The probes having the nucleotide sequence represented by SEQ ID NOS: 107870, 107871, 107872, 107873, 107874, 107875, 107876, 107877, 107878, 107879, 107880 were selected from the ORFs having the nucleotide sequence represented by SEQ ID NO: 77588.

The probes having the nucleotide sequence represented by SEQ ID NOS: 107881, 107882, 107883, 107884, 107885, 107886, 107887, 107888, 107889, 107890, 107891 were selected from the ORFs having the nucleotide sequence represented by SEQ ID NO: 77589.

The probes having the nucleotide sequence represented by SEQ ID NOS: 107892, 107893, 107894, 107895, 107896, 107897, 107898, 107899, 107900, 107901, 107902 were selected from the ORFs having the nucleotide sequence represented by SEQ ID NO: 77590.

The probes having the nucleotide sequence represented by SEQ ID NOS: 107903, 107904, 107905, 107906, 107907, 107908, 107909, 107910, 107911, 107912, 107913 were selected from the ORFs having the nucleotide sequence represented by SEQ ID NO: 77591.

The probes having the nucleotide sequence represented by SEQ ID NOS: 107914, 107915, 107916, 107917, 107918, 107919, 107920, 107921, 107922, 107923, 107924 were selected from the ORFs having the nucleotide sequence represented by SEQ ID NO: 77592.

The probes having the nucleotide sequence represented by SEQ ID NOS: 107925, 107926, 107927, 107928, 107929, 107930, 107931, 107932, 107933, 107934, 107935 were selected from the ORFs having the nucleotide sequence represented by SEQ ID NO: 77593.

The probes having the nucleotide sequence represented by SEQ ID NOS: 107936, 107937, 107938, 107939, 107940, 107941, 107942, 107943, 107944, 107945, 107946 were selected from the ORFs having the nucleotide sequence represented by SEQ ID NO: 77594.

The probes having the nucleotide sequence represented by SEQ ID NOS: 107947, 107948, 107949, 107950, 107951, 107952, 107953, 107954, 107955, 107956, 107957 were selected from the ORFs having the nucleotide sequence represented by SEQ ID NO: 77595.

The probes having the nucleotide sequence represented by SEQ ID NOS: 107958, 107959, 107960, 107961, 107962, 107963, 107964, 107965, 107966, 107967, 107968 were selected from the ORFs having the nucleotide sequence represented by SEQ ID NO: 77596.

The probes having the nucleotide sequence represented by SEQ ID NOS: 107969, 107970, 107971, 107972, 107973, 107974, 107975, 107976, 107977, 107978, 107979 were selected from the ORFs having the nucleotide sequence represented by SEQ ID NO: 77597.

The probes having the nucleotide sequence represented by SEQ ID NOS: 107980, 107981, 107982, 107983, 107984, 107985, 107986, 107987, 107988, 107989, 107990 were selected from the ORFs having the nucleotide sequence represented by SEQ ID NO: 77598.

The probes having the nucleotide sequence represented by SEQ ID NOS: 107991, 107992, 107993, 107994, 107995, 107996, 107997, 107998, 107999, 108000, 108001 were selected from the ORFs having the nucleotide sequence represented by SEQ ID NO: 77599.

The probes having the nucleotide sequence represented by SEQ ID NOS: 108002, 108003, 108004, 108005, 108006, 108007, 108008, 108009, 108010, 108011, 108012 were selected from the ORFs having the nucleotide sequence represented by SEQ ID NO: 77600.

The probes having the nucleotide sequence represented by SEQ ID NOS: 108013, 108014, 108015, 108016, 108017, 108018, 108019, 108020, 108021, 108022, 108023 were selected from the ORFs having the nucleotide sequence represented by SEQ ID NO: 77601.

The probes having the nucleotide sequence represented by SEQ ID NOS: 108024, 108025, 108026, 108027, 108028, 108029, 108030, 108031, 108032, 108033, 108034 were selected from the ORFs having the nucleotide sequence represented by SEQ ID NO: 77602.

The probes having the nucleotide sequence represented by SEQ ID NOS: 108035, 108036, 108037, 108038, 108039, 108040, 108041, 108042, 108043, 108044, 108045 were selected from the ORFs having the nucleotide sequence represented by SEQ ID NO: 77603.

The probes having the nucleotide sequence represented by SEQ ID NOS: 108046, 108047, 108048, 108049, 108050, 108051, 108052, 108053, 108054, 108055, 108056 were selected from the ORFs having the nucleotide sequence represented by SEQ ID NO: 77604.

The probes having the nucleotide sequence represented by SEQ ID NOS: 108057, 108058, 108059, 108060, 108061, 108062, 108063, 108064, 108065, 108066, 108067 were selected from the ORFs having the nucleotide sequence represented by SEQ ID NO: 77605.

The probes having the nucleotide sequence represented by SEQ ID NOS: 108068, 108069, 108070, 108071, 108072, 108073, 108074, 108075, 108076, 108077, 108078 were selected from the ORFs having the nucleotide sequence represented by SEQ ID NO: 77606.

The probes having the nucleotide sequence represented by SEQ ID NOS: 108079, 108080, 108081, 108082, 108083, 108084, 108085, 108086, 108087, 108088, 108089 were selected from the ORFs having the nucleotide sequence represented by SEQ ID NO: 77607.

The probes having the nucleotide sequence represented by SEQ ID NOS: 108090, 108091, 108092, 108093, 108094, 108095, 108096, 108097, 108098, 108099, 108100 were selected from the ORFs having the nucleotide sequence represented by SEQ ID NO: 77608.

The probes having the nucleotide sequence represented by SEQ ID NOS: 108101, 108102, 108103, 108104, 108105, 108106, 108107, 108108, 108109, 108110, 108111 were selected from the ORFs having the nucleotide sequence represented by SEQ ID NO: 77609.

The probes having the nucleotide sequence represented by SEQ ID NOS: 108112, 108113, 108114, 108115, 108116, 108117, 108118, 108119, 108120, 108121, 108122 were selected from the ORFs having the nucleotide sequence represented by SEQ ID NO: 77610.

The probes having the nucleotide sequence represented by SEQ ID NOS: 108123, 108124, 108125, 108126, 108127, 108128, 108129, 108130, 108131, 108132, 108133 were selected from the ORFs having the nucleotide sequence represented by SEQ ID NO: 77611.

The probes having the nucleotide sequence represented by SEQ ID NOS: 108134, 108135, 108136, 108137, 108138, 108139, 108140, 108141, 108142, 108143, 108144 were selected from the ORFs having the nucleotide sequence represented by SEQ ID NO: 77612.

The probes having the nucleotide sequence represented by SEQ ID NOS: 108145, 108146, 108147, 108148, 108149, 108150, 108151, 108152, 108153, 108154, 108155 were selected from the ORFs having the nucleotide sequence represented by SEQ ID NO: 77613.

The probes having the nucleotide sequence represented by SEQ ID NOS: 108156, 108157, 108158, 108159, 108160, 108161, 108162, 108163, 108164, 108165, 108166 were selected from the ORFs having the nucleotide sequence represented by SEQ ID NO: 77614.

The probes having the nucleotide sequence represented by SEQ ID NOS: 108167, 108168, 108169, 108170, 108171, 108172, 108173, 108174, 108175, 108176, 108177 were selected from the ORFs having the nucleotide sequence represented by SEQ ID NO: 77615.

The probes having the nucleotide sequence represented by SEQ ID NOS: 108178, 108179, 108180, 108181, 108182, 108183, 108184, 108185, 108186, 108187, 108188 were selected from the ORFs having the nucleotide sequence represented by SEQ ID NO: 77616.

The probes having the nucleotide sequence represented by SEQ ID NOS: 108189, 108190, 108191, 108192, 108193, 108194, 108195, 108196, 108197, 108198, 108199 were selected from the ORFs having the nucleotide sequence represented by SEQ ID NO: 77617.

The probes having the nucleotide sequence represented by SEQ ID NOS: 108200, 108201, 108202, 108203, 108204, 108205, 108206, 108207, 108208, 108209, 108210 were selected from the ORFs having the nucleotide sequence represented by SEQ ID NO: 77618.

The probes having the nucleotide sequence represented by SEQ ID NOS: 108211, 108212, 108213, 108214, 108215, 108216, 108217, 108218, 108219, 108220, 108221 were selected from the ORFs having the nucleotide sequence represented by SEQ ID NO: 77619.

The probes having the nucleotide sequence represented by SEQ ID NOS: 108222, 108223, 108224, 108225, 108226, 108227, 108228, 108229, 108230, 108231, 108232 were selected from the ORFs having the nucleotide sequence represented by SEQ ID NO: 77620.

The probes having the nucleotide sequence represented by SEQ ID NOS: 108233, 108234, 108235, 108236, 108237, 108238, 108239, 108240, 108241, 108242, 108243 were selected from the ORFs having the nucleotide sequence represented by. SEQ ID NO: 77621.

The probes having the nucleotide sequence represented by SEQ ID NOS: 108244, 108245, 108246, 108247, 108248, 108249, 108250, 108251, 108252, 108253, 108254 were selected from the ORFs having the nucleotide sequence represented by SEQ ID NO: 77622.

The probes having the nucleotide sequence represented by SEQ ID NOS: 108255, 108256, 108257, 108258, 108259, 108260, 108261, 108262, 108263, 108264, 108265, 108266, 108267, 108268, 108269, 108270, 108271, 108272, 108273, 108274, 108275, 108276 were selected from the ORFs having the nucleotide sequence represented by SEQ ID NO: 77623.

The probes having the nucleotide sequence represented by SEQ ID NOS: 108277, 108278, 108279, 108280, 108281, 108282, 108283, 108284, 108285, 108286, 108287 were selected from the ORFs having the nucleotide sequence represented by SEQ ID NO: 77624.

The probes having the nucleotide sequence represented by SEQ ID NOS: 108288, 108289, 108290, 108291, 108292, 108293, 108294, 108295, 108296, 108297, 108298 were selected from the ORFs having the nucleotide sequence represented by SEQ ID NO: 77625.

The probes having the nucleotide sequence represented by SEQ ID NOS: 108299, 108300, 108301, 108302, 108303, 108304, 108305, 108306, 108307, 108308, 108309 were selected from the ORFs having the nucleotide sequence represented by SEQ ID NO: 77626.

The probes having the nucleotide sequence represented by SEQ ID NOS: 108310, 108311, 108312, 108313, 108314, 108315, 108316, 108317, 108318, 108319, 108320 were selected from the ORFs having the nucleotide sequence represented by SEQ ID NO: 77627.

The probes having the nucleotide sequence represented by SEQ ID NOS: 108321, 108322, 108323, 108324, 108325, 108326, 108327, 108328, 108329, 108330, 108331 were selected from the ORFs having the nucleotide sequence represented by SEQ ID NO: 77628.

The probes having the nucleotide sequence represented by SEQ ID NOS: 108332, 108333, 108334, 108335, 108336, 108337, 108338, 108339, 108340, 108341, 108342 were selected from the ORFs having the nucleotide sequence represented by SEQ ID NO: 77629.

The probes having the nucleotide sequence represented by SEQ ID NOS: 108343, 108344, 108345, 108346, 108347, 108348, 108349, 108350, 108351, 108352, 108353 were selected from the ORFs having the nucleotide sequence represented by SEQ ID NO: 77630.

The probes having the nucleotide sequence represented by SEQ ID NOS: 108354, 108355, 108356, 108357, 108358, 108359, 108360, 108361, 108362, 108363, 108364 were selected from the ORFs having the nucleotide sequence represented by SEQ ID NO: 77631.

The probes having the nucleotide sequence represented by SEQ ID NOS: 108365, 108366, 108367, 108368, 108369, 108370, 108371, 108372, 108373, 108374, 108375 were selected from the ORFs having the nucleotide sequence represented by SEQ ID NO: 77632.

The probes having the nucleotide sequence represented by SEQ ID NOS: 108376, 108377, 108378, 108379, 108380, 108381, 108382, 108383, 108384, 108385, 108386 were selected from the ORFs having the nucleotide sequence represented by SEQ ID NO: 77633.

The probes having the nucleotide sequence represented by SEQ ID NOS: 108387, 108388, 108389, 108390, 108391, 108392, 108393, 108394, 108395, 108396, 108397 were selected from the ORFs having the nucleotide sequence represented by SEQ ID NO: 77634.

The probes having the nucleotide sequence represented by SEQ ID NOS: 108398, 108399, 108400, 108401, 108402, 108403, 108404, 108405, 108406, 108407, 108408 were selected from the ORFs having the nucleotide sequence represented by SEQ ID NO: 77635.

The probes having the nucleotide sequence represented by SEQ ID NOS: 108409, 108410, 108411, 108412, 108413, 108414, 108415, 108416, 108417, 108418, 108419 were selected from the ORFs having the nucleotide sequence represented by SEQ ID NO: 77636.

The probes having the nucleotide sequence represented by SEQ ID NOS: 108420, 108421, 108422, 108423, 108424, 108425, 108426, 108427, 108428, 108429, 108430 were selected from the ORFs having the nucleotide sequence represented by SEQ ID NO: 77637.

The probes having the nucleotide sequence represented by SEQ ID NOS: 108431, 108432, 108433, 108434, 108435, 108436, 108437, 108438, 108439, 108440, 108441 were selected from the ORFs having the nucleotide sequence represented by SEQ ID NO: 77638.

The probes having the nucleotide sequence represented by SEQ ID NOS: 108442, 108443, 108444, 108445, 108446, 108447, 108448, 108449, 108450, 108451, 108452 were selected from the ORFs having the nucleotide sequence represented by SEQ ID NO: 77639.

The probes having the nucleotide sequence represented by SEQ ID NOS: 108453, 108454, 108455, 108456, 108457, 108458, 108459, 108460, 108461, 108462, 108463 were selected from the ORFs having the nucleotide sequence represented by SEQ ID NO: 77640.

The probes having the nucleotide sequence represented by SEQ ID NOS: 108464, 108465, 108466, 108467, 108468, 108469, 108470, 108471, 108472, 108473, 108474 were selected from the ORFs having the nucleotide sequence represented by SEQ ID NO: 77641.

The probes having the nucleotide sequence represented by SEQ ID NOS: 108475, 108476, 108477, 108478, 108479, 108480, 108481, 108482, 108483, 108484, 108485 were selected from the ORFs having the nucleotide sequence represented by SEQ ID NO: 77642.

The probes having the nucleotide sequence represented by SEQ ID NOS: 108486, 108487, 108488, 108489, 108490, 108491, 108492, 108493, 108494, 108495, 108496 were selected from the ORFs having the nucleotide sequence represented by SEQ ID NO: 77643.

The probes having the nucleotide sequence represented by SEQ ID NOS: 108497, 108498, 108499, 108500, 108501, 108502, 108503, 108504, 108505, 108506, 108507 were selected from the ORFs having the nucleotide sequence represented by SEQ ID NO: 77644.

The probes having the nucleotide sequence represented by SEQ ID NOS: 108508, 108509, 108510, 108511, 108512, 108513, 108514, 108515, 108516, 108517, 108518 were selected from the ORFs having the nucleotide sequence represented by SEQ ID NO: 77645.

The probes having the nucleotide sequence represented by SEQ ID NOS: 108519, 108520, 108521, 108522, 108523, 108524, 108525, 108526, 108527, 108528, 108529 were selected from the ORFs having the nucleotide sequence represented by SEQ ID NO: 77646.

The probes having the nucleotide sequence represented by SEQ ID NOS: 108530, 108531, 108532, 108533, 108534, 108535, 108536, 108537, 108538, 108539, 108540 were selected from the ORFs having the nucleotide sequence represented by SEQ ID NO: 77647.

The probes having the nucleotide sequence represented by SEQ ID NOS: 108541, 108.542, 108543, 108544, 108545, 108546, 108547, 108548, 108549, 108550, 108551 were selected from the ORFs having the nucleotide sequence represented by SEQ ID NO: 77648.

The probes having the nucleotide sequence represented by SEQ ID NOS: 108552, 108553, 108554, 108555, 108556, 108557, 108558, 108559, 108560, 108561, 108562 were selected from the ORFs having the nucleotide sequence represented by SEQ ID NO: 77649.

The probes having the nucleotide sequence represented by SEQ ID NOS: 108563, 108564, 108565, 108566, 108567, 108568, 108569, 108570, 108571, 108572, 108573 were selected from the ORFs having the nucleotide sequence represented by SEQ ID NO: 77650.

The probes having the nucleotide sequence represented by SEQ ID NOS: 108574, 108575, 108576, 108577, 108578, 108579, 108580, 108581, 108582, 108583, 108584 were selected from the ORFs having the nucleotide sequence represented by SEQ ID NO: 77651.

The probes having the nucleotide sequence represented by SEQ ID NOS: 108585, 108586, 108587, 108588, 108589, 108590, 108591, 108592, 108593, 108594, 108595 were selected from the ORFs having the nucleotide sequence represented by SEQ ID NO: 77652.

The probes having the nucleotide sequence represented by SEQ ID NOS: 108596, 108597, 108598, 108599, 108600, 108601, 108602, 108603, 108604, 108605, 108606 were selected from the ORFs having the nucleotide sequence represented by SEQ ID NO: 77653.

The probes having the nucleotide sequence represented by SEQ ID NOS: 108607, 108608, 108609, 108610, 108611, 108612, 108613, 108614, 108615, 108616, 108617 were selected from the ORFs having the nucleotide sequence represented by SEQ ID NO: 77654.

The probes having the nucleotide sequence represented by SEQ ID NOS: 108618, 108619, 108620, 108621, 108622, 108623, 108624, 108625, 108626, 108627, 108628 were selected from the ORFs having the nucleotide sequence represented by SEQ ID NO: 77655.

The probes having the nucleotide sequence represented by SEQ ID NOS: 108629, 108630, 108631, 108632, 108633, 108634, 108635, 108636, 108637, 108638, 108639 were selected from the ORFs having the nucleotide sequence represented by SEQ ID NO: 77656.

The probes having the nucleotide sequence represented by SEQ ID NOS: 108640, 108641, 108642, 108643, 108644, 108645, 108646, 108647, 108648, 108649, 108650 were selected from the ORFs having the nucleotide sequence represented by SEQ ID NO: 77657.

The probes having the nucleotide sequence represented by SEQ ID NOS: 108651, 108652, 108653, 108654, 108655, 108656, 108657, 108658, 108659, 108660, 108661 were selected from the ORFs having the nucleotide sequence represented by SEQ ID NO: 77658.

The probes having the nucleotide sequence represented by SEQ ID NOS: 108662, 108663, 108664, 108665, 108666, 108667, 108668, 108669, 108670, 108671, 108672 were selected from the ORFs having the nucleotide sequence represented by SEQ ID NO: 77659.

The probes having the nucleotide sequence represented by SEQ ID NOS: 108673, 108674, 108675, 108676, 108677, 108678, 108679, 108680, 108681, 108682, 108683 were selected from the ORFs having the nucleotide sequence represented by SEQ ID NO: 77660.

The probes having the nucleotide sequence represented by SEQ ID NOS: 108684, 108685, 108686, 108687, 108688, 108689, 108690, 108691, 108692, 108693, 108694 were selected from the ORFs having the nucleotide sequence represented by SEQ ID NO: 77661.

The probes having the nucleotide sequence represented by SEQ ID NOS: 108695, 108696, 108697, 108698, 108699, 108700, 108701, 108702, 108703, 108704, 108705 were selected from the ORFs having the nucleotide sequence represented by SEQ ID NO: 77662.

The probes having the nucleotide sequence represented by SEQ ID NOS: 108706, 108707, 108708, 108709, 108710, 108711, 108712, 108713, 108714, 108715, 108716 were selected from the ORFs having the nucleotide sequence represented by SEQ ID NO: 77663.

The probes having the nucleotide sequence represented by SEQ ID NOS: 108717, 108718, 108719, 108720, 108721, 108722, 108723, 108724, 108725, 108726, 108727 were selected from the ORFs having the nucleotide sequence represented by SEQ ID NO: 77664.

The probes having the nucleotide sequence represented by SEQ ID NOS: 108728, 108729, 108730, 108731, 108732, 108733, 108734, 108735, 108736, 108737, 108738 were selected from the ORFs having the nucleotide sequence represented by SEQ ID NO: 77665.

The probes having the nucleotide sequence represented by SEQ ID NOS: 108739, 108740, 108741, 108742, 108743, 108744, 108745, 108746, 108747, 108748, 108749 were selected from the ORFs having the nucleotide sequence represented by SEQ ID NO: 77666.

The probes having the nucleotide sequence represented by SEQ ID NOS: 108750, 108751, 108752, 108753, 108754, 108755, 108756, 108757, 108758, 108759, 108760 were selected from the ORFs having the nucleotide sequence represented by SEQ ID NO: 77667.

The probes having the nucleotide sequence represented by SEQ ID NOS: 108761, 108762, 108763, 108764, 108765, 108766, 108767, 108768, 108769, 108770, 108771 were selected from the ORFs having the nucleotide sequence represented by SEQ ID NO: 77668.

The probes having the nucleotide sequence represented by SEQ ID NOS: 108772, 108773, 108774, 108775, 108776, 108777, 108778, 108779, 108780, 108781, 108782 were selected from the ORFs having the nucleotide sequence represented by SEQ ID NO: 77669.

The probes having the nucleotide sequence represented by SEQ ID NOS: 108783, 108784, 108785, 108786, 108787, 108788, 108789, 108790, 108791, 108792, 108793 were selected from the ORFs having the nucleotide sequence represented by SEQ ID NO: 77670.

The probes having the nucleotide sequence represented by SEQ ID NOS: 108794, 108795, 108796, 108797, 108798, 108799, 108800, 108801, 108802, 108803, 108804 were selected from the ORFs having the nucleotide sequence represented by SEQ ID NO: 77671.

The probes having the nucleotide sequence represented by SEQ ID NOS: 108805, 108806, 108807, 108808, 108809, 108810, 108811, 108812, 108813, 108814, 108815 were selected from the ORFs having the nucleotide sequence represented by SEQ ID NO: 77672.

The probes having the nucleotide sequence represented by SEQ ID NOS: 108816, 108817, 108818, 108819, 108820, 108821, 108822, 108823, 108824, 108825, 108826 were selected from the ORFs having the nucleotide sequence represented by SEQ ID NO: 77673.

The probes having the nucleotide sequence represented by SEQ ID NOS: 108827, 108828, 108829, 108830, 108831, 108832, 108833, 108834, 108835, 108836, 108837 were selected from the ORFs having the nucleotide sequence represented by SEQ ID NO: 77674.

The probes having the nucleotide sequence represented by SEQ ID NOS: 108838, 108839, 108840, 108841, 108842, 108843, 108844, 108845, 108846, 108847, 108848 were selected from the ORFs having the nucleotide sequence represented by SEQ ID NO: 77675.

The probes having the nucleotide sequence represented by SEQ ID NOS: 108849, 108850, 108851, 108852, 108853, 108854, 108855, 108856, 108857, 108858, 108859 were selected from the ORFs having the nucleotide sequence represented by SEQ ID NO: 77676.

The probes having the nucleotide sequence represented by SEQ ID NOS: 108860, 108861, 108862, 108863, 108864, 108865, 108866, 108867, 108868, 108869, 108870 were selected from the ORFs having the nucleotide sequence represented by SEQ ID NO: 77677.

The probes having the nucleotide sequence represented by SEQ ID NOS: 108871, 108872, 108873, 108874, 108875, 108876, 108877, 108878, 108879, 108880, 108881 were selected from the ORFs having the nucleotide sequence represented by SEQ ID NO: 77678.

The probes having the nucleotide sequence represented by SEQ ID NOS: 108882, 108883, 108884, 108885, 108886, 108887, 108888, 108889, 108890, 10889.1, 108892 were selected from the ORFs having the nucleotide sequence represented by SEQ ID NO: 77679.

The probes having the nucleotide sequence represented by SEQ ID NOS: 108893, 108894, 108895, 108896, 108897, 108898, 108899, 108900, 108901, 108902, 108903 were selected from the ORFs having the nucleotide sequence represented by SEQ ID NO: 77680.

The probes having the nucleotide sequence represented by SEQ ID NOS: 108904, 108905, 108906, 108907, 108908, 108909, 108910, 108911, 108912, 108913, 108914 were selected from the ORFs having the nucleotide sequence represented by SEQ ID NO: 77681.

The probes having the nucleotide sequence represented by SEQ ID NOS: 108915, 108916, 108917, 108918, 108919, 108920, 108921, 108922, 108923, 108924, 108925 were selected from the ORFs having the nucleotide sequence represented by SEQ ID NO: 77682.

The probes having the nucleotide sequence represented by SEQ ID NOS: 108926, 108927, 108928, 108929, 108930, 108931, 108932, 108933, 108934, 108935, 108936 were selected from the ORFs having the nucleotide sequence represented by SEQ ID NO: 77683.

The probes having the nucleotide sequence represented by SEQ ID NOS: 108937, 108938, 108939, 108940, 108941, 108942, 108943, 108944, 108945, 108946, 108947 were selected from the ORFs having the nucleotide sequence represented by SEQ ID NO: 77684.

The probes having the nucleotide sequence represented by SEQ ID NOS: 108948, 108949, 108950, 108951, 108952, 108953, 108954, 108955, 108956, 108957, 108958 were selected from the ORFs having the nucleotide sequence represented by SEQ ID NO: 77685.

The probes having the nucleotide sequence represented by SEQ ID NOS: 108959, 108960, 108961, 108962, 108963, 108964, 108965, 108966, 108967, 108968, 108969 were selected from the ORFs having the nucleotide sequence represented by SEQ ID NO: 77686.

The probes having the nucleotide sequence represented by SEQ ID NOS: 108970, 108971, 108972, 108973, 108974, 108975, 108976, 108977, 108978, 108979, 108980 were selected from the ORFs having the nucleotide sequence represented by SEQ ID NO: 77687.

The probes having the nucleotide sequence represented by SEQ ID NOS: 108981, 108982, 108983, 108984, 108985, 108986, 108987, 108988, 108989, 108990, 108991 were selected from the ORFs having the nucleotide sequence represented by SEQ ID NO: 77688.

The probes having the nucleotide sequence represented by SEQ ID NOS: 108992, 108993, 108994, 108995, 108996, 108997, 108998, 108999, 109000, 109001, 109002 were selected from the ORFs having the nucleotide sequence represented by SEQ ID NO: 77689.

The probes having the nucleotide sequence represented by SEQ ID NOS: 109003, 109004, 109005, 109006, 109007, 109008, 109009, 109010, 109011, 109012, 109013 were selected from the ORFs having the nucleotide sequence represented by SEQ ID NO: 77690.

The probes having the nucleotide sequence represented by SEQ ID NOS: 109014, 109015, 109016, 109017, 109018, 109019, 109020, 109021, 109022, 109023, 109024 were selected from the ORFs having the nucleotide sequence represented by SEQ ID NO: 77691.

The probes having the nucleotide sequence represented by SEQ ID NOS: 109025, 109026, 109027, 109028, 109029, 109030, 109031, 109032, 109033, 109034, 109035 were selected from the ORFs having the nucleotide sequence represented by SEQ ID NO: 77692.

The probes having the nucleotide sequence represented by SEQ ID NOS: 109036, 109037, 109038, 109039, 109040, 109041, 109042, 109043, 109044, 109045, 109046, 109047, 109048, 109049, 109050, 109051, 109052, 109053, 109054, 109055, 109056, 109057 were selected from the ORFs having the nucleotide sequence represented by SEQ ID NO: 77693.

The probes having the nucleotide sequence represented by SEQ ID NOS: 109058, 109059, 109060, 109061, 109062, 109063, 109064, 109065, 109066, 109067, 109068 were selected from the ORFs having the nucleotide sequence represented by SEQ ID NO: 77694.

The probes having the nucleotide sequence represented by SEQ ID NOS: 109069, 109070, 109071, 109072, 109073, 109074, 109075, 109076, 109077, 109078, 109079 were selected from the ORFs having the nucleotide sequence represented by SEQ ID NO: 77695.

The probes having the nucleotide sequence represented by SEQ ID NOS: 109080, 109081, 109082, 109083, 109084, 109085, 109086, 109087, 109088, 109089, 109090 were selected from the ORFs having the nucleotide sequence represented by SEQ ID NO: 77696.

The probes having the nucleotide sequence represented by SEQ ID NOS: 109091, 109092, 109093, 109094, 109095, 109096, 109097, 109098, 109099, 109100, 109101 were selected from the ORFs having the nucleotide sequence represented by SEQ ID NO: 77697.

The probes having the nucleotide sequence represented by SEQ ID NOS: 109102, 109103, 109104, 109105, 109106, 109107, 109108, 109109, 109110, 109111, 109112 were selected from the ORFs having the nucleotide sequence represented by SEQ ID NO: 77698.

The probes having the nucleotide sequence represented by SEQ ID NOS: 109113, 109114, 109115, 109116, 109117, 109118, 109119, 109120, 109121, 109122, 109123 were selected from the ORFs having the nucleotide sequence represented by SEQ ID NO: 77699.

The probes having the nucleotide sequence represented by SEQ ID NOS: 109124, 109125, 109126, 109127, 109128, 109129, 109130, 109131, 109132, 109133, 109134 were selected from the ORFs having the nucleotide sequence represented by SEQ ID NO: 77700.

The probes having the nucleotide sequence represented by SEQ ID NOS: 109135, 109136, 109137, 109138, 109139, 109140, 109141, 109142, 109143, 109144, 109145 were selected from the ORFs having the nucleotide sequence represented by SEQ ID NO: 77701.

The probes having the nucleotide sequence represented by SEQ ID NOS: 109146, 109147, 109148, 109149, 109150, 109151, 109152, 109153, 109154, 109155, 109156 were selected from the ORFs having the nucleotide sequence represented by SEQ ID NO: 77702.

The probes having the nucleotide sequence represented by SEQ ID NOS: 109157, 109158, 109159, 109160, 109161, 109162, 109163, 109164, 109165, 109166, 109167 were selected from the ORFs having the nucleotide sequence represented by SEQ ID NO: 77703.

The probes having the nucleotide sequence represented by SEQ ID NOS: 109168, 109169, 109170, 109171, 109172, 109173, 109174, 109175, 109176, 109177, 109178 were selected from the ORFs having the nucleotide sequence represented by SEQ ID NO: 77704.

The probes having the nucleotide sequence represented by SEQ ID NOS: 109179, 109180, 109181, 109182, 109183, 109184, 109185, 109186, 109187, 109188, 109189 were selected from the ORFs having the nucleotide sequence represented by SEQ ID NO: 77705.

The probes having the nucleotide sequence represented by SEQ ID NOS: 109190, 109191, 109192, 109193, 109194, 109195, 109196, 109197, 109198, 109199, 109200 were selected from the ORFs having the nucleotide sequence represented by SEQ ID NO: 77706.

The probes having the nucleotide sequence represented by SEQ ID NOS: 109201, 109202, 109203, 109204, 109205, 109206, 109207, 109208, 109209, 109210, 109211 were selected from the ORFs having the nucleotide sequence represented by SEQ ID NO: 77707.

The probes having the nucleotide sequence represented by SEQ ID NOS: 109212, 109213, 109214, 109215, 109216, 109217, 109218, 109219, 109220, 109221, 109222 were selected from the ORFs having the nucleotide sequence represented by SEQ ID NO: 77708.

The probes having the nucleotide sequence represented by SEQ ID NOS: 109223, 109224, 109225, 109226, 109227, 109228, 109229, 109230, 109231, 109232, 109233 were selected from the ORFs having the nucleotide sequence represented by SEQ ID NO: 77709.

The probes having the nucleotide sequence represented by SEQ ID NOS: 109234, 109235, 109236, 109237, 109238, 109239, 109240, 109241, 109242, 109243, 109244 were selected from the ORFs having the nucleotide sequence represented by SEQ ID NO: 77710.

The probes having the nucleotide sequence represented by SEQ ID NOS: 109245, 109246, 109247, 109248, 109249, 109250, 109251, 109252, 109253, 109254, 109255 were selected from the ORFs having the nucleotide sequence represented by SEQ ID NO: 77711.

The probes having the nucleotide sequence represented by SEQ ID NOS: 109256, 109257, 109258, 109259, 109260, 109261, 109262, 109263, 109264, 109265, 109266 were selected from the ORFs having the nucleotide sequence represented by SEQ ID NO: 77712.

The probes having the nucleotide sequence represented by SEQ ID NOS: 109267, 109268, 109269, 109270, 109271, 109272, 109273, 109274, 109275, 109276, 109277 were selected from the ORFs having the nucleotide sequence represented by SEQ ID NO: 77713.

The probes having the nucleotide sequence represented by SEQ ID NOS: 109278, 109279, 109280, 109281, 109282, 109283, 109284, 109285, 109286, 109287, 109288 were selected from the ORFs having the nucleotide sequence represented by SEQ ID NO: 77714.

The probes having the nucleotide sequence represented by SEQ ID NOS: 109289, 109290, 109291, 109292, 109293, 109294, 109295, 109296, 109297, 109298, 109299 were selected from the ORFs having the nucleotide sequence represented by SEQ ID NO: 77715.

The probes having the nucleotide sequence represented by SEQ ID NOS: 109300, 109301, 109302, 109303, 109304, 109305, 109306, 109307, 109308, 109309, 109310 were selected from the ORFs having the nucleotide sequence represented by SEQ ID NO: 77716.

The probes having the nucleotide sequence represented by SEQ ID NOS: 109311, 109312, 109313, 109314, 109315, 109316, 109317, 109318, 109319, 109320, 109321 were selected from the ORFs having the nucleotide sequence represented by SEQ ID NO: 77717.

The probes having the nucleotide sequence represented by SEQ ID NOS: 109322, 109323, 109324, 109325, 109326, 109327, 109328, 109329, 109330, 109331, 109332 were selected from the ORFs having the nucleotide sequence represented by SEQ ID NO: 77718.

The probes having the nucleotide sequence represented by SEQ ID NOS: 109333, 109334, 109335, 109336, 109337, 109338, 109339, 109340, 109341, 109342, 109343 were selected from the ORFs having the nucleotide sequence represented by SEQ ID NO: 77719.

The probes having the nucleotide sequence represented by SEQ ID NOS: 109344, 109345, 109346, 109347, 109348, 109349, 109350, 109351, 109352, 109353, 109354 were selected from the ORFs having the nucleotide sequence represented by SEQ ID NO: 77720.

The probes having the nucleotide sequence represented by SEQ ID NOS: 109355, 109356, 109357, 109358, 109359, 109360, 109361, 109362, 109363, 109364, 109365 were selected from the ORFs having the nucleotide sequence represented by SEQ ID NO: 77721.

The probes having the nucleotide sequence represented by SEQ ID NOS: 109366, 109367, 109368, 109369, 109370, 109371, 109372, 109373, 109374, 109375, 109376 were selected from the ORFs having the nucleotide sequence represented by SEQ ID NO: 77722.

The probes having the nucleotide sequence represented by SEQ ID NOS: 109377, 109378, 109379, 109380, 109381, 109382, 109383, 109384, 109385, 109386, 109387 were selected from the ORFs having the nucleotide sequence represented by SEQ ID NO: 77723.

The probes having the nucleotide sequence represented by SEQ ID NOS: 109388, 109389, 109390, 109391, 109392, 109393, 109394, 109395, 109396, 109397, 109398 were selected from the ORFs having the nucleotide sequence represented by SEQ ID NO: 77724.

The probes having the nucleotide sequence represented by SEQ ID NOS: 109399, 109400, 109401, 109402, 109403, 109404, 109405, 109406, 109407, 109408, 109409 were selected from the ORFs having the nucleotide sequence represented by SEQ ID NO: 77725.

The probes having the nucleotide sequence represented by SEQ ID NOS: 109410, 109411, 109412, 109413, 109414, 109415, 109416, 109417, 109418, 109419, 109420 were selected from the ORFs having the nucleotide sequence represented by SEQ ID NO: 77726.

The probes having the nucleotide sequence represented by SEQ ID NOS: 109421, 109422, 109423, 109424, 109425, 109426, 109427, 109428, 109429, 109430, 109431 were selected from the ORFs having the nucleotide sequence represented by SEQ ID NO: 77727.

The probes having the nucleotide sequence represented by SEQ ID NOS: 109432, 109433, 109434, 109435, 109436, 109437, 109438, 109439, 109440, 109441, 109442 were selected from the ORFs having the nucleotide sequence represented by SEQ ID NO: 77728.

The probes having the nucleotide sequence represented by SEQ ID NOS: 109443, 109444, 109445, 109446, 109447, 109448, 109449, 109450, 109451, 109452, 109453 were selected from the ORFs having the nucleotide sequence represented by SEQ ID NO: 77729.

The probes having the nucleotide sequence represented by SEQ ID NOS: 109454, 109455, 109456, 109457, 109458, 109459, 109460, 109461, 109462, 109463, 109464 were selected from the ORFs having the nucleotide sequence represented by SEQ ID NO: 77730.

The probes having the nucleotide sequence represented by SEQ ID NOS: 109465, 109466, 109467, 109468, 109469, 109470, 109471, 109472, 109473, 109474, 109475 were selected from the ORFs having the nucleotide sequence represented by SEQ ID NO: 77731.

The probes having the nucleotide sequence represented by SEQ ID NOS: 109476, 109477, 109478, 109479, 109480, 109481, 109482, 109483, 109484, 109485, 109486 were selected from the ORFs having the nucleotide sequence represented by SEQ ID NO: 77732.

The probes having the nucleotide sequence represented by SEQ ID NOS: 109487, 109488, 109489, 109490, 109491, 109492, 109493, 109494, 109495, 109496, 109497 were selected from the ORFs having the nucleotide sequence represented by SEQ ID NO: 77733.

The probes having the nucleotide sequence represented by SEQ ID NOS: 109498, 109499, 109500, 109501, 109502, 109503, 109504, 109505, 109506, 109507, 109508 were selected from the ORFs having the nucleotide sequence represented by SEQ ID NO: 77734.

The probes having the nucleotide sequence represented by SEQ ID NOS: 109509, 109510, 109511, 109512, 109513, 109514, 109515, 109516, 109517, 109518, 109519 were selected from the ORFs having the nucleotide sequence represented by SEQ ID NO: 77735.

The probes having the nucleotide sequence represented by SEQ ID NOS: 109520, 109521, 109522, 109523, 109524, 109525, 109526, 109527, 109528, 109529, 109530, 109531, 109532, 109533, 109534, 109535, 109536, 109537, 109538, 109539, 109540, 109541 were selected from the ORFs having the nucleotide sequence represented by SEQ ID NO: 77736.

The probes having the nucleotide sequence represented by SEQ ID NOS: 109542, 109543, 109544, 109545, 109546, 109547, 109548, 109549, 109550, 109551, 109552 were selected from the ORFs having the nucleotide sequence represented by SEQ ID NO: 77737.

The probes having the nucleotide sequence represented by SEQ ID NOS: 109553, 109554, 109555, 109556, 109557, 109558, 109559, 109560, 109561, 109562, 109563 were selected from the ORFs having the nucleotide sequence represented by SEQ ID NO: 77738.

The probes having the nucleotide sequence represented by SEQ ID NOS: 109564, 109565, 109566, 109567, 109568, 109569, 109570, 109571, 109572, 109573, 109574 were selected from the ORFs having the nucleotide sequence represented by SEQ ID NO: 77739.

The probes having the nucleotide sequence represented by SEQ ID NOS: 109575, 109576, 109577, 109578, 109579, 109580, 109581, 109582, 109583, 109584, 109585 were selected from the ORFs having the nucleotide sequence represented by SEQ ID NO: 77740.

The probes having the nucleotide sequence represented by SEQ ID NOS: 109586, 109587, 109588, 109589, 109590, 109591, 109592, 109593, 109594, 109595, 109596 were selected from the ORFs having the nucleotide sequence represented by SEQ ID NO: 77741.

The probes having the nucleotide sequence represented by SEQ ID NOS: 109597, 109598, 109599, 109600, 109601, 109602, 109603, 109604, 109605, 109606, 109607 were selected from the ORFs having the nucleotide sequence represented by SEQ ID NO: 77742.

The probes having the nucleotide sequence represented by SEQ ID NOS: 109608, 109609, 109610, 109611, 109612, 109613, 109614, 109615, 109616, 109617, 109618 were selected from the ORFs having the nucleotide sequence represented by SEQ ID NO: 77743.

The probes having the nucleotide sequence represented by SEQ ID NOS: 109619, 109620, 109621, 109622, 109623, 109624, 109625, 109626, 109627, 109628, 109629 were selected from the ORFs having the nucleotide sequence represented by SEQ ID NO: 77744.

The probes having the nucleotide sequence represented by SEQ ID NOS: 109630, 109631, 109632, 109633, 109634, 109635, 109636, 109637, 109638, 109639, 109640 were selected from the ORFs having the nucleotide sequence represented by SEQ ID NO: 77745.

The probes having the nucleotide sequence represented by SEQ ID NOS: 109641, 109642, 109643, 109644, 109645, 109646, 109647, 109648, 109649, 109650, 109651 were selected from the ORFs having the nucleotide sequence represented by SEQ ID NO: 77746.

The probes having the nucleotide sequence represented by SEQ ID NOS: 109652, 109653, 109654, 109655, 109656, 109657, 109658, 109659, 109660, 109661, 109662 were selected from the ORFs having the nucleotide sequence represented by SEQ ID NO: 77747.

The probes having the nucleotide sequence represented by SEQ ID NOS: 109663, 109664, 109665, 109666, 109667, 109668, 109669, 109670, 109671, 109672, 109673 were selected from the ORFs having the nucleotide sequence represented by SEQ ID NO: 77748.

The probes having the nucleotide sequence represented by SEQ ID NOS: 109674, 109675, 109676, 109677, 109678, 109679, 109680, 109681, 109682, 109683, 109684 were selected from the ORFs having the nucleotide sequence represented by SEQ ID NO: 77749.

The probes having the nucleotide sequence represented by SEQ ID NOS: 109685, 109686, 109687, 109688, 109689, 109690, 109691, 109692, 109693, 109694, 109695 were selected from the ORFs having the nucleotide sequence represented by SEQ ID NO: 77750.

The probes having the nucleotide sequence represented by SEQ ID NOS: 109696, 109697, 109698, 109699, 109700, 109701, 109702, 109703, 109704, 109705, 109706 were selected from the ORFs having the nucleotide sequence represented by SEQ ID NO: 77751.

The probes having the nucleotide sequence represented by SEQ ID NOS: 109707, 109708, 109709, 109710, 109711, 109712, 109713, 109714, 109715, 109716, 109717 were selected from the ORFs having the nucleotide sequence represented by SEQ ID NO: 77752.

The probes having the nucleotide sequence represented by SEQ ID NOS: 109718, 109719, 109720, 109721, 109722, 109723, 109724, 109725, 109726, 109727, 109728 were selected from the ORFs having the nucleotide sequence represented by SEQ ID NO: 77753.

The probes having the nucleotide sequence represented by SEQ ID NOS: 109729, 109730, 109731, 109732, 109733, 109734, 109735, 109736, 109737, 109738, 109739 were selected from the ORFs having the nucleotide sequence represented by SEQ ID NO: 77754.

The probes having the nucleotide sequence represented by SEQ ID NOS: 109740, 109741, 109742, 109743, 109744, 109745, 109746, 109747, 109748, 109749, 109750 were selected from the ORFs having the nucleotide sequence represented by SEQ ID NO: 77755.

The probes having the nucleotide sequence represented by SEQ ID NOS: 109751, 109752, 109753, 109754, 109755, 109756, 109757, 109758, 109759, 109760, 109761 were selected from the ORFs having the nucleotide sequence represented by SEQ ID NO: 77756.

The probes having the nucleotide sequence represented by SEQ ID NOS: 109762, 109763, 109764, 109765, 109766, 109767, 109768, 109769, 109770, 109771, 109772 were selected from the ORFs having the nucleotide sequence represented by SEQ ID NO: 77757.

The probes having the nucleotide sequence represented by SEQ ID NOS: 109773, 109774, 109775, 109776, 109777, 109778, 109779, 109780, 109781, 109782, 109783 were selected from the ORFs having the nucleotide sequence represented by SEQ ID NO: 77758.

The probes having the nucleotide sequence represented by SEQ ID NOS: 109784, 109785, 109786, 109787, 109788, 109789, 109790, 109791, 109792, 109793, 109794 were selected from the ORFs having the nucleotide sequence represented by SEQ ID NO: 77759.

The probes having the nucleotide sequence represented by SEQ ID NOS: 109795, 109796, 109797, 109798, 109799, 109800, 109801, 109802, 109803, 109804, 109805 were selected from the ORFs having the nucleotide sequence represented by SEQ ID NO: 77760.

The probes having the nucleotide sequence represented by SEQ ID NOS: 109806, 109807, 109808, 109809, 109810, 109811, 109812, 109813, 109814, 109815, 109816 were selected from the ORFs having the nucleotide sequence represented by SEQ ID NO: 77761.

The probes having the nucleotide sequence represented by SEQ ID NOS: 109817, 109818, 109819, 109820, 109821, 109822, 109823, 109824, 109825, 109826, 109827 were selected from the ORFs having the nucleotide sequence represented by SEQ ID NO: 77762.

The probes having the nucleotide sequence represented by SEQ ID NOS: 109828, 109829, 109830, 109831, 109832, 109833, 109834, 109835, 109836, 109837, 109838 were selected from the ORFs having the nucleotide sequence represented by SEQ ID NO: 77763.

The probes having the nucleotide sequence represented by SEQ ID NOS: 109839, 109840, 109841, 109842, 109843, 109844, 109845, 109846, 109847, 109848, 109849 were selected from the ORFs having the nucleotide sequence represented by SEQ ID NO: 77764.

The probes having the nucleotide sequence represented by SEQ ID NOS: 109850, 109851, 109852, 109853, 109854, 109855, 109856, 109857, 109858, 109859, 109860, 109861, 109862, 109863, 109864, 109865, 109866, 109867, 109868, 109869, 109870, 109871 were selected from the ORFs having the nucleotide sequence represented by SEQ ID NO: 77765.

The probes having the nucleotide sequence represented by SEQ ID NOS: 109872, 109873, 109874, 109875, 109876, 109877, 109878, 109879, 109880, 109881, 109882 were selected from the ORFs having the nucleotide sequence represented by SEQ ID NO: 77766.

The probes having the nucleotide sequence represented by SEQ ID NOS: 109883, 109884, 109885, 109886, 109887, 109888, 109889, 109890, 109891, 109892, 109893 were selected from the ORFs having the nucleotide sequence represented by SEQ ID NO: 77767.

The probes having the nucleotide sequence represented by SEQ ID NOS: 109894, 109895, 109896, 109897, 109898, 109899, 109900, 109901, 109902, 109903, 109904 were selected from the ORFs having the nucleotide sequence represented by SEQ ID NO: 77768.

The probes having the nucleotide sequence represented by SEQ ID NOS: 109905, 109906, 109907, 109908, 109909, 109910, 109911, 109912, 109913, 109914, 109915 were selected from the ORFs having the nucleotide sequence represented by SEQ ID NO: 77769.

The probes having the nucleotide sequence represented by SEQ ID NOS: 109916, 109917, 109918, 109919, 109920, 109921, 109922, 109923, 109924, 109925, 109926 were selected from the ORFs having the nucleotide sequence represented by SEQ ID NO: 77770.

The probes having the nucleotide sequence represented by SEQ ID NOS: 109927, 109928, 109929, 109930, 109931, 109932, 109933, 109934, 109935, 109936, 109937, 109938, 109939, 109940, 109941, 109942, 109943, 109944, 109945, 109946, 109947, 109948 were selected from the ORFs having the nucleotide sequence represented by SEQ ID NO: 77771.

The probes having the nucleotide sequence represented by SEQ ID NOS: 109949, 109950, 109951, 109952, 109953, 109954, 109955, 109956, 109957, 109958, 109959 were selected from the ORFs having the nucleotide sequence represented by SEQ ID NO: 77772.

The probes having the nucleotide sequence represented by SEQ ID NOS: 109960, 109961, 109962, 109963, 109964, 109965, 109966, 109967, 109968, 109969, 109970 were selected from the ORFs having the nucleotide sequence represented by SEQ ID NO: 77773.

The probes having the nucleotide sequence represented by SEQ ID NOS: 109971, 109972, 109973, 109974, 109975, 109976, 109977, 109978, 109979, 109980, 109981 were selected from the ORFs having the nucleotide sequence represented by SEQ ID NO: 77774.

The probes having the nucleotide sequence represented by SEQ ID NOS: 109982, 109983, 109984, 109985, 109986, 109987, 109988, 109989, 109990, 109991, 109992 were selected from the ORFs having the nucleotide sequence represented by SEQ ID NO: 77775.

The probes having the nucleotide sequence represented by SEQ ID NOS: 109993, 109994, 109995, 109996, 109997, 109998, 109999, 110000, 110001, 110002, 110003 were selected from the ORFs having the nucleotide sequence represented by SEQ ID NO: 77776.

The probes having the nucleotide sequence represented by SEQ ID NOS: 110004, 110005, 110006, 110007, 110008, 110009, 110010, 110011, 110012, 110013, 110014 were selected from the ORFs having the nucleotide sequence represented by SEQ ID NO: 77777.

The probes having the nucleotide sequence represented by SEQ ID NOS: 110015, 110016, 110017, 110018, 110019, 110020, 110021, 110022, 110023, 110024, 110025 were selected from the ORFs having the nucleotide sequence represented by SEQ ID NO: 77778.

The probes having the nucleotide sequence represented by SEQ ID NOS: 110026, 110027, 110028, 110029, 110030, 110031, 110032, 110033, 110034, 110035, 110036 were selected from the ORFs having the nucleotide sequence represented by SEQ ID NO: 77779.

The probes having the nucleotide sequence represented by SEQ ID NOS: 110037, 110038, 110039, 110040, 110041, 110042, 110043, 110044, 110045, 110046, 110047 were selected from the ORFs having the nucleotide sequence represented by SEQ ID NO: 77780.

The probes having the nucleotide sequence represented by SEQ ID NOS: 110048, 110049, 110050, 110051, 110052, 110053, 110054, 110055, 110056, 110057, 110058 were selected from the ORFs having the nucleotide sequence represented by SEQ ID NO: 77781.

The probes having the nucleotide sequence represented by SEQ ID NOS: 110059, 110060, 110061, 110062, 110063, 110064, 110065, 110066, 110067, 110068, 110069 were selected from the ORFs having the nucleotide sequence represented by SEQ ID NO: 77782.

The probes having the nucleotide sequence represented by SEQ ID NOS: 110070, 110071, 110072, 110073, 110074, 110075, 110076, 110077, 110078, 110079, 110080 were selected from the ORFs having the nucleotide sequence represented by SEQ ID NO: 77783.

The probes having the nucleotide sequence represented by SEQ ID NOS: 110081, 110082, 110083, 110084, 110085, 110086, 110087, 110088, 110089, 110090, 110091 were selected from the ORFs having the nucleotide sequence represented by SEQ ID NO: 77784.

The probes having the nucleotide sequence represented by SEQ ID NOS: 110092, 110093, 110094, 110095, 110096, 110097, 110098, 110099, 110100, 110101, 110102 were selected from the ORFs having the nucleotide sequence represented by SEQ ID NO: 77785.

The probes having the nucleotide sequence represented by SEQ ID NOS: 110103, 110104, 110105, 110106, 110107, 110108, 110109, 110110, 110111, 110112, 110113 were selected from the ORFs having the nucleotide sequence represented by SEQ ID NO: 77786.

The probes having the nucleotide sequence represented by SEQ ID NOS: 110114, 110115, 110116, 110117, 110118, 110119, 110120, 110121, 110122, 110123, 110124 were selected from the ORFs having the nucleotide sequence represented by SEQ ID NO: 77787.

The probes having the nucleotide sequence represented by SEQ ID NOS: 110125, 110126, 110127, 110128, 110129, 110130, 110131, 110132, 110133, 110134, 110135 were selected from the ORFs having the nucleotide sequence represented by SEQ ID NO: 77788.

The probes having the nucleotide sequence represented by SEQ ID NOS: 110136, 110137, 110138, 110139, 110140, 110141, 110142, 110143, 110144, 110145, 110146 were selected from the ORFs having the nucleotide sequence represented by SEQ ID NO: 77789.

The probes having the nucleotide sequence represented by SEQ ID NOS: 110147, 110148, 110149, 110150, 110151, 110152, 110153, 110154, 110155, 110156, 110157 were selected from the ORFs having the nucleotide sequence represented by SEQ ID NO: 77790.

The probes having the nucleotide sequence represented by SEQ ID NOS: 110158, 110159, 110160, 110161, 110162, 110163, 110164, 110165, 110166, 110167, 110168 were selected from the ORFs having the nucleotide sequence represented by SEQ ID NO: 77791.

The probes having the nucleotide sequence represented by SEQ ID NOS: 110169, 110170, 110171, 110172, 110173, 110174, 110175, 110176, 110177, 110178, 110179 were selected from the ORFs having the nucleotide sequence represented by SEQ ID NO: 77792.

The probes having the nucleotide sequence represented by SEQ ID NOS: 110180, 110181, 110182, 110183, 110184, 110185, 110186, 110187, 110188, 110189, 110190 were selected from the ORFs having the nucleotide sequence represented by SEQ ID NO: 77793.

The probes having the nucleotide sequence represented by SEQ ID NOS: 110191, 110192, 110193, 110194, 110195, 110196, 110197, 110198, 110199, 110200, 110201 were selected from the ORFs having the nucleotide sequence represented by SEQ ID NO: 77794.

The probes having the nucleotide sequence represented by SEQ ID NOS: 110202, 110203, 110204, 110205, 110206, 110207, 110208, 110209, 110210, 110211, 110212 were selected from the ORFs having the nucleotide sequence represented by SEQ ID NO: 77795.

The probes having the nucleotide sequence represented by SEQ ID NOS: 110213, 110214, 110215, 110216, 110217, 110218, 110219, 110220, 110221, 110222, 110223 were selected from the ORFs having the nucleotide sequence represented by SEQ ID NO: 77796.

The probes having the nucleotide sequence represented by SEQ ID NOS: 110224, 110225, 110226, 110227, 110228, 110229, 110230, 110231, 110232, 110233, 110234 were selected from the ORFs having the nucleotide sequence represented by SEQ ID NO: 77797.

The probes having the nucleotide sequence represented by SEQ ID NOS: 110235, 110236, 110237, 110238, 110239, 110240, 110241, 110242, 110243, 110244, 110245 were selected from the ORFs having the nucleotide sequence represented by SEQ ID NO: 77798.

The probes having the nucleotide sequence represented by SEQ ID NOS: 110246, 110247, 110248, 110249, 110250, 110251, 110252, 110253, 110254, 110255, 110256 were selected from the ORFs having the nucleotide sequence represented by SEQ ID NO: 77799.

The probes having the nucleotide sequence represented by SEQ ID NOS: 110257, 110258, 110259, 110260, 110261, 110262, 110263, 110264, 110265, 110266, 110267 were selected from the ORFs having the nucleotide sequence represented by SEQ ID NO: 77800.

The probes having the nucleotide sequence represented by SEQ ID NOS: 110268, 110269, 110270, 110271, 110272, 110273, 110274, 110275, 110276, 110277, 110278 were selected from the ORFs having the nucleotide sequence represented by SEQ ID NO: 77801.

The probes having the nucleotide sequence represented by SEQ ID NOS: 110279, 110280, 110281, 110282, 110283, 110284, 110285, 110286, 110287, 110288, 110289 were selected from the ORFs having the nucleotide sequence represented by SEQ ID NO: 77802.

The probes having the nucleotide sequence represented by SEQ ID NOS: 110290, 110291, 110292, 110293, 110294, 110295, 110296, 110297, 110298, 110299, 110300 were selected from the ORFs having the nucleotide sequence represented by SEQ ID NO: 77803.

The probes having the nucleotide sequence represented by SEQ ID NOS: 110301, 110302, 110303, 110304, 110305, 110306, 110307, 110308, 110309, 110310, 110311 were selected from the ORFs having the nucleotide sequence represented by SEQ ID NO: 77804.

The probes having the nucleotide sequence represented by SEQ ID NOS: 110312, 110313, 110314, 110315, 110316, 110317, 110318, 110319, 110320, 110321, 110322 were selected from the ORFs having the nucleotide sequence represented by SEQ ID NO: 77805.

The probes having the nucleotide sequence represented by SEQ ID NOS: 110323, 110324, 110325, 110326, 110327, 110328, 110329, 110330, 110331, 110332, 110333 were selected from the ORFs having the nucleotide sequence represented by SEQ ID NO: 77806.

The probes having the nucleotide sequence represented by SEQ ID NOS: 110334, 110335, 110336, 110337, 110338, 110339, 110340, 110341, 110342, 110343, 110344 were selected from the ORFs having the nucleotide sequence represented by SEQ ID NO: 77807.

The probes having the nucleotide sequence represented by SEQ ID NOS: 110345, 110346, 110347, 110348, 110349, 110350, 110351, 110352, 110353, 110354, 110355 were selected from the ORFs having the nucleotide sequence represented by SEQ. ID NO: 77808.

The probes having the nucleotide sequence represented by SEQ ID NOS: 110356, 110357, 110358, 110359, 110360, 110361, 110362, 110363, 110364, 110365, 110366 were selected from the ORFs having the nucleotide sequence represented by SEQ ID NO: 77809.

The probes having the nucleotide sequence represented by SEQ ID NOS: 110367, 110368, 110369, 110370, 110371, 110372, 110373, 110374, 110375, 110376, 110377 were selected from the ORFs having the nucleotide sequence represented by SEQ ID NO: 77810.

The probes having the nucleotide sequence represented by SEQ ID NOS: 110378, 110379, 110380, 110381, 110382, 110383, 110384, 110385, 110386, 110387, 110388, 110389, 110390, 110391, 110392, 110393, 110394, 110395, 110396, 110397, 110398, 110399 were selected from the ORFs having the nucleotide sequence represented by SEQ ID NO: 77811.

The probes having the nucleotide sequence represented by SEQ ID NOS: 110400, 110401, 110402, 110403, 110404, 110405, 110406, 110407, 110408, 110409, 110410 were selected from the ORFs having the nucleotide sequence represented by SEQ ID NO: 77812.

The probes having the nucleotide sequence represented by SEQ ID NOS: 110411, 110412, 110413, 110414, 110415, 110416, 110417, 110418, 110419, 110420, 110421 were selected from the ORFs having the nucleotide sequence represented by SEQ ID NO: 77813.

The probes having the nucleotide sequence represented by SEQ ID NOS: 110422, 110423, 110424, 110425, 110426, 110427, 110428, 110429, 110430, 110431, 110432 were selected from the ORFs having the nucleotide sequence represented by SEQ ID NO: 77814.

The probes having the nucleotide sequence represented by SEQ ID NOS: 110433, 110434, 110435, 110436, 110437, 110438, 110439, 110440, 110441, 110442, 110443 were selected from the ORFs having the nucleotide sequence represented by SEQ ID NO: 77815.

The probes having the nucleotide sequence represented by SEQ ID NOS: 110444, 110445, 110446, 110447, 110448, 110449, 110450, 110451, 110452, 110453, 110454 were selected from the ORFs having the nucleotide sequence represented by SEQ ID NO: 77816.

The probes having the nucleotide sequence represented by SEQ ID NOS: 110455, 110456, 110457, 110458, 110459, 110460, 110461, 110462, 110463, 110464, 110465 were selected from the ORFs having the nucleotide sequence represented by SEQ ID NO: 77817.

The probes having the nucleotide sequence represented by SEQ ID NOS: 110466, 110467, 110468, 110469, 110470, 110471, 110472, 110473, 110474, 110475, 110476 were selected from the ORFs having the nucleotide sequence represented by SEQ ID NO: 77818.

The probes having the nucleotide sequence represented by SEQ ID NOS: 110477, 110478, 110479, 110480, 110481, 110482, 110483, 110484, 110485, 110486, 110487 were selected from the ORFs having the nucleotide sequence represented by SEQ ID NO: 77819.

The probes having the nucleotide sequence represented by SEQ ID NOS: 110488, 110489, 110490, 110491, 110492, 110493, 110494, 110495, 110496, 110497, 110498 were selected from the ORFs having the nucleotide sequence represented by SEQ ID NO: 77820.

The probes having the nucleotide sequence represented by SEQ ID NOS: 110499, 110500, 110501, 110502, 110503, 110504, 110505, 110506, 110507, 110508, 110509 were selected from the ORFs having the nucleotide sequence represented by SEQ ID NO: 77821.

The probes having the nucleotide sequence represented by SEQ ID NOS: 110510, 110511, 110512, 110513, 110514, 110515, 110516, 110517, 110518, 110519, 110520 were selected from the ORFs having the nucleotide sequence represented by SEQ ID NO: 77822.

The probes having the nucleotide sequence represented by SEQ ID NOS: 110521, 110522, 110523, 110524, 110525, 110526, 110527, 110528, 110529, 110530, 110531 were selected from the ORFs having the nucleotide sequence represented by SEQ ID NO: 77823.

The probes having the nucleotide sequence represented by SEQ ID NOS: 110532, 110533, 110534, 110535, 110536, 110537, 110538, 110539, 110540, 110541, 110542 were selected from the ORFs having the nucleotide sequence represented by SEQ ID NO: 77824.

The probes having the nucleotide sequence represented by SEQ ID NOS: 110543, 110544, 110545, 110546, 110547, 110548, 110549, 110550, 110551, 110552, 110553 were selected from the ORFs having the nucleotide sequence represented by SEQ ID NO: 77825.

The probes having the nucleotide sequence represented by SEQ ID NOS: 110554, 110555, 110556, 110557, 110558, 110559, 110560, 110561, 110562, 110563, 110564 were selected from the ORFs having the nucleotide sequence represented by SEQ ID NO: 77826.

The probes having the nucleotide sequence represented by SEQ ID NOS: 110565, 110566, 110567, 110568, 110569, 110570, 110571, 110572, 110573, 110574, 110575 were selected from the ORFs having the nucleotide sequence represented by SEQ ID NO: 77827.

The probes having the nucleotide sequence represented by SEQ ID NOS: 110576, 110577, 110578, 110579, 110580, 110581, 110582, 110583, 110584, 110585, 110586 were selected from the ORFs having the nucleotide sequence represented by SEQ ID NO: 77828.

The probes having the nucleotide sequence represented by SEQ ID NOS: 110587, 110588, 110589, 110590, 110591, 110592, 110593, 110594, 110595, 110596, 110597 were selected from the ORFs having the nucleotide sequence represented by SEQ ID NO: 77829.

The probes having the nucleotide sequence represented by SEQ ID NOS: 110598, 110599, 110600, 110601, 110602, 110603, 110604, 110605, 110606, 110607, 110608 were selected from the ORFs having the nucleotide sequence represented by SEQ ID NO: 77830.

The probes having the nucleotide sequence represented by SEQ ID NOS: 110609, 110610, 110611, 110612, 110613, 110614, 110615, 110616, 110617, 110618, 110619 were selected from the ORFs having the nucleotide sequence represented by SEQ ID NO: 77831.

The probes having the nucleotide sequence represented by SEQ ID NOS: 110620, 110621, 110622, 110623, 110624, 110625, 110626, 110627, 110628, 110629, 110630 were selected from the ORFs having the nucleotide sequence represented by SEQ ID NO: 77832.

The probes having the nucleotide sequence represented by SEQ ID NOS: 110631, 110632, 110633, 110634, 110635, 110636, 110637, 110638, 110639, 110640, 110641 were selected from the ORFs having the nucleotide sequence represented by SEQ ID NO: 77833.

The probes having the nucleotide sequence represented by SEQ ID NOS: 110642, 110643, 110644, 110645, 110646, 110647, 110648, 110649, 110650, 110651, 110652, 110653, 110654, 110655, 110656, 110657, 110658, 110659, 110660, 110661, 110662, 110663 were selected from the ORFs having the nucleotide sequence represented by SEQ ID NO: 77834.

The probes having the nucleotide sequence represented by SEQ ID NOS: 110664, 110665, 110666, 110667, 110668, 110669, 110670, 110671, 110672, 110673, 110674 were selected from the ORFs having the nucleotide sequence represented by SEQ ID NO: 77835.

The probes having the nucleotide sequence represented by SEQ ID NOS: 110675, 110676, 110677, 110678, 110679, 110680, 110681, 110682, 110683, 110684, 110685 were selected from the ORFs having the nucleotide sequence represented by SEQ ID NO: 77836.

The probes having the nucleotide sequence represented by SEQ ID NOS: 110686, 110687, 110688, 110689, 110690, 110691, 110692, 110693, 110694, 110695, 110696 were selected from the ORFs having the nucleotide sequence represented by SEQ ID NO: 77837.

The probes having the nucleotide sequence represented by SEQ ID NOS: 110697, 110698, 110699, 110700, 110701, 110702, 110703, 110704, 110705, 110706, 110707 were selected from the ORFs having the nucleotide sequence represented by SEQ ID NO: 77838.

The probes having the nucleotide sequence represented by SEQ ID NOS: 110708, 110709, 110710, 110711, 110712, 110713, 110714, 110715, 110716, 110717, 110718 were selected from the ORFs having the nucleotide sequence represented by SEQ ID NO: 77839.

The probes having the nucleotide sequence represented by SEQ ID NOS: 110719, 110720, 110721, 110722, 110723, 110724, 110725, 110726, 110727, 110728, 110729 were selected from the ORFs having the nucleotide sequence represented by SEQ ID NO: 77840.

The probes having the nucleotide sequence represented by SEQ ID NOS: 110730, 110731, 110732, 110733, 110734, 110735, 110736, 110737, 110738, 110739, 110740 were selected from the ORFs having the nucleotide sequence represented by SEQ ID NO: 77841.

The probes having the nucleotide sequence represented by SEQ ID NOS: 110741, 110742, 110743, 110744, 110745, 110746, 110747, 110748, 110749, 110750, 110751 were selected from the ORFs having the nucleotide sequence represented by SEQ ID NO: 77842.

The probes having the nucleotide sequence represented by SEQ ID NOS: 110752, 110753, 110754, 110755, 110756, 110757, 110758, 110759, 110760, 110761, 110762 were selected from the ORFs having the nucleotide sequence represented by SEQ ID NO: 77843.

The probes having the nucleotide sequence represented by SEQ ID NOS: 110763, 110764, 110765, 110766, 110767, 110768, 110769, 110770, 110771, 110772, 110773 were selected from the ORFs having the nucleotide sequence represented by SEQ ID NO: 77844.

The probes having the nucleotide sequence represented by SEQ ID NOS: 110774, 110775, 110776, 110777, 110778, 110779, 110780, 110781, 110782, 110783, 110784, 110785, 110786, 110787, 110788, 110789, 110790, 110791, 110792, 110793, 110794, 110795 were selected from the ORFs having the nucleotide sequence represented by SEQ ID NO: 77845.

The probes having the nucleotide sequence represented by SEQ ID NOS: 110796, 110797, 110798, 110799, 110800, 110801, 110802, 110803, 110804, 110805, 110806 were selected from the ORFs having the nucleotide sequence represented by SEQ ID NO: 77846.

The probes having the nucleotide sequence represented by SEQ ID NOS: 110807, 110808, 110809, 110810, 110811, 110812, 110813, 110814, 110815, 110816, 110817 were selected from the ORFs having the nucleotide sequence represented by SEQ ID NO: 77847.

The probes having the nucleotide sequence represented by SEQ ID NOS: 110818, 110819, 110820, 110821, 110822, 110823, 110824, 110825, 110826, 110827, 110828 were selected from the ORFs having the nucleotide sequence represented by SEQ ID NO: 77848.

The probes having the nucleotide sequence represented by SEQ ID NOS: 110829, 110830, 110831, 110832, 110833, 110834, 110835, 110836, 110837, 110838, 110839 were selected from the ORFs having the nucleotide sequence represented by SEQ ID NO: 77849.

The probes having the nucleotide sequence represented by SEQ ID NOS: 110840, 110841, 110842, 110843, 110844, 110845, 110846, 110847, 110848, 110849, 110850 were selected from the ORFs having the nucleotide sequence represented by SEQ ID NO: 77850.

The probes having the nucleotide sequence represented by SEQ ID NOS: 110851, 110852, 110853, 110854, 110855, 110856, 110857, 110858, 110859, 110860, 110861 were selected from the ORFs having the nucleotide sequence represented by SEQ ID NO: 77851.

The probes having the nucleotide sequence represented by SEQ ID NOS: 110862, 110863, 110864, 110865, 110866, 110867, 110868, 110869, 110870, 110871, 110872 were selected from the ORFs having the nucleotide sequence represented by SEQ ID NO: 77852.

The probes having the nucleotide sequence represented by SEQ ID NOS: 110873, 110874, 110875, 110876, 110877, 110878, 110879, 110880, 110881, 110882, 110883 were selected from the ORFs having the nucleotide sequence represented by SEQ ID NO: 77853.

The probes having the nucleotide sequence represented by SEQ ID NOS: 110884, 110885, 110886, 110887, 110888, 110889, 110890, 110891, 110892, 110893, 110894 were selected from the ORFs having the nucleotide sequence represented by SEQ ID NO: 77854.

The probes having the nucleotide sequence represented by SEQ ID NOS: 110895, 110896, 110897, 110898, 110899, 110900, 110901, 110902, 110903, 110904, 110905 were selected from the ORFs having the nucleotide sequence represented by SEQ ID NO: 77855.

The probes having the nucleotide sequence represented by SEQ ID NOS: 110906, 110907, 110908, 110909, 110910, 110911, 110912, 110913, 110914, 110915, 110916 were selected from the ORFs having the nucleotide sequence represented by SEQ ID NO: 77856.

The probes having the nucleotide sequence represented by SEQ ID NOS: 110917, 110918, 110919, 110920, 110921, 110922, 110923, 110924, 110925, 110926, 110927 were selected from the ORFs having the nucleotide sequence represented by SEQ ID NO: 77857.

The probes having the nucleotide sequence represented by SEQ ID NOS: 110928, 110929, 110930, 110931, 110932, 110933, 110934, 110935, 110936, 110937, 110938 were selected from the ORFs having the nucleotide sequence represented by SEQ ID NO: 77858.

The probes having the nucleotide sequence represented by SEQ ID NOS: 110939, 110940, 110941, 110942, 110943, 110944, 110945, 110946, 110947, 110948, 110949 were selected from the ORFs having the nucleotide sequence represented by SEQ ID NO: 77859.

The probes having the nucleotide sequence represented by SEQ ID NOS: 110950, 110951, 110952, 110953, 110954, 110955, 110956, 110957, 110958, 110959, 110960 were selected from the ORFs having the nucleotide sequence represented by SEQ ID NO: 77860.

The probes having the nucleotide sequence represented by SEQ ID NOS: 110961, 110962, 110963, 110964, 110965, 110966, 110967, 110968, 110969, 110970, 110971 were selected from the ORFs having the nucleotide sequence represented by SEQ ID NO: 77861.

The probes having the nucleotide sequence represented by SEQ ID NOS: 110972, 110973, 110974, 110975, 110976, 110977, 110978, 110979, 110980, 110981, 110982 were selected from the ORFs having the nucleotide sequence represented by SEQ ID NO: 77862.

The probes having the nucleotide sequence represented by SEQ ID NOS: 110983, 110984, 110985, 110986, 110987, 110988, 110989, 110990, 110991, 110992, 110993 were selected from the ORFs having the nucleotide sequence represented by SEQ ID NO: 77863.

The probes having the nucleotide sequence represented by SEQ ID NOS: 110994, 110995, 110996, 110997, 110998, 110999, 111000, 111001, 111002, 111003, 111004 were selected from the ORFs having the nucleotide sequence represented by SEQ ID NO: 77864.

The probes having the nucleotide sequence represented by SEQ ID NOS: 111005, 111006, 111007, 111008, 111009, 111010, 111011, 111012, 111013, 111014, 111015 were selected from the ORFs having the nucleotide sequence represented by SEQ ID NO: 77865.

The probes having the nucleotide sequence represented by SEQ ID NOS: 111016, 111017, 111018, 111019, 111020, 111021, 111022, 111023, 111024, 111025, 111026 were selected from the ORFs having the nucleotide sequence represented by SEQ ID NO: 77866.

The probes having the nucleotide sequence represented by SEQ ID NOS: 111027, 111028, 111029, 111030, 111031, 111032, 111033, 111034, 111035, 111036, 111037 were selected from the ORFs having the nucleotide sequence represented by SEQ ID NO: 77867.

The probes having the nucleotide sequence represented by SEQ ID NOS: 111038, 111039, 111040, 111041, 111042, 111043, 111044, 111045, 111046, 111047, 111048 were selected from the ORFs having the nucleotide sequence represented by SEQ ID NO: 77868.

The probes having the nucleotide sequence represented by SEQ ID NOS: 111049, 111050, 111051, 111052, 111053, 111054, 111055, 111056, 111057, 111058, 111059 were selected from the ORFs having the nucleotide sequence represented by SEQ ID NO: 77869.

The probes having the nucleotide sequence represented by SEQ ID NOS: 111060, 111061, 111062, 111063, 111064, 111065, 111066, 111067, 111068, 111069, 111070 were selected from the ORFs having the nucleotide sequence represented by SEQ ID NO: 77870.

The probes having the nucleotide sequence represented by SEQ ID NOS: 111071, 111072, 111073, 111074, 111075, 111076, 111077, 111078, 111079, 111080, 111081 were selected from the ORFs having the nucleotide sequence represented by SEQ ID NO: 77871.

The probes having the nucleotide sequence represented by SEQ ID NOS: 111082, 111083, 111084, 111085, 111086, 111087, 111088, 111089, 111090, 111091, 111092 were selected from the ORFs having the nucleotide sequence represented by SEQ ID NO: 77872.

The probes having the nucleotide sequence represented by SEQ ID NOS: 111093, 111094, 111095, 111096, 111097, 111098, 111099, 111100, 111101, 111102, 111103 were selected from the ORFs having the nucleotide sequence represented by SEQ ID NO: 77873.

The probes having the nucleotide sequence represented by SEQ ID NOS: 111104, 111105, 111106, 111107, 111108, 111109, 111110, 111111, 111112, 111113, 111114 were selected from the ORFs having the nucleotide sequence represented by SEQ ID NO: 77874.

The probes having the nucleotide sequence represented by SEQ ID NOS: 111115, 111116, 111117, 111118, 111119, 111120, 111121, 111122, 111123, 111124, 111125 were selected from the ORFs having the nucleotide sequence represented by SEQ ID NO: 77875.

The probes having the nucleotide sequence represented by SEQ ID NOS: 111126, 111127, 111128, 111129, 111130, 111131, 111132, 111133, 111134, 111135, 111136 were selected from the ORFs having the nucleotide sequence represented by SEQ ID NO: 77876.

The probes having the nucleotide sequence represented by SEQ ID NOS: 111137, 111138, 111139, 111140, 111141, 111142, 111143, 111144, 111145, 111146, 111147 were selected from the ORFs having the nucleotide sequence represented by SEQ ID NO: 77877.

The probes having the nucleotide sequence represented by SEQ ID NOS: 111148, 111149, 111150, 111151, 111152, 111153, 111154, 111155, 111156, 111157, 111158 were selected from the ORFs having the nucleotide sequence represented by SEQ ID NO: 77878.

The probes having the nucleotide sequence represented by SEQ ID NOS: 111159, 111160, 111161, 111162, 111163, 111164, 111165, 111166, 111167, 111168, 111169 were selected from the ORFs having the nucleotide sequence represented by SEQ ID NO: 77879.

The probes having the nucleotide sequence represented by SEQ ID NOS: 111170, 111171, 111172, 111173, 111174, 111175, 111176, 111177, 111178, 111179, 111180 were selected from the ORFs having the nucleotide sequence represented by SEQ ID NO: 77880.

The probes having the nucleotide sequence represented by SEQ ID NOS: 111181, 111182, 111183, 111184, 111185, 111186, 111187, 111188, 111189, 111190, 111191 were selected from the ORFs having the nucleotide sequence represented by SEQ ID NO: 77881.

The probes having the nucleotide sequence represented by SEQ ID NOS: 111192, 111193, 111194, 111195, 111196, 111197, 111198, 111199, 111200, 111201, 111202 were selected from the ORFs having the nucleotide sequence represented by SEQ ID NO: 77882.

The probes having the nucleotide sequence represented by SEQ ID NOS: 111203, 111204, 111205, 111206, 111207, 111208, 111209, 111210, 111211, 111212, 111213 were selected from the ORFs having the nucleotide sequence represented by SEQ ID NO: 77883.

The probes having the nucleotide sequence represented by SEQ ID NOS: 111214, 111215, 111216, 111217, 111218, 111219, 111220, 111221, 111222, 111223, 111224 were selected from the ORFs having the nucleotide sequence represented by SEQ ID NO: 77884.

The probes having the nucleotide sequence represented by SEQ ID NOS: 111225, 111226, 111227, 111228, 111229, 111230, 111231, 111232, 111233, 111234, 111235 were selected from the ORFs having the nucleotide sequence represented by SEQ ID NO: 77885.

The probes having the nucleotide sequence represented by SEQ ID NOS: 111236, 111237, 111238, 111239, 111240, 111241, 111242, 111243, 111244, 111245, 111246 were selected from the ORFs having the nucleotide sequence represented by SEQ ID NO: 77886.

The probes having the nucleotide sequence represented by SEQ ID NOS: 111247, 111248, 111249, 111250, 111251, 111252, 111253, 111254, 111255, 111256, 111257 were selected from the ORFs having the nucleotide sequence represented by SEQ ID NO: 77887.

The probes having the nucleotide sequence represented by SEQ ID NOS: 111258, 111259, 111260, 111261, 111262, 111263, 111264, 111265, 111266, 111267, 111268 were selected from the ORFs having the nucleotide sequence represented by SEQ ID NO: 77888.

The probes having the nucleotide sequence represented by SEQ ID NOS: 111269, 111270, 111271, 111272, 111273, 111274, 111275, 111276, 111277, 111278, 111279 were selected from the ORFs having the nucleotide sequence represented by SEQ ID NO: 77889.

The probes having the nucleotide sequence represented by SEQ ID NOS: 111280, 111281, 111282, 111283, 111284, 111285, 111286, 111287, 111288, 111289, 111290 were selected from the ORFs having the nucleotide sequence represented by SEQ ID NO: 77890.

The probes having the nucleotide sequence represented by SEQ ID NOS: 111291, 111292, 111293, 111294, 111295, 111296, 111297, 111298, 111299, 111300, 111301 were selected from the ORFs having the nucleotide sequence represented by SEQ ID NO: 77891.

The probes having the nucleotide sequence represented by SEQ ID NOS: 111302, 111303, 111304, 111305, 111306, 111307, 111308, 111309, 111310, 111311, 111312 were selected from the ORFs having the nucleotide sequence represented by SEQ ID NO: 77892.

The probes having the nucleotide sequence represented by SEQ ID NOS: 111313, 111314, 111315, 111316, 111317, 111318, 111319, 111320, 111321, 111322, 111323 were selected from the ORFs having the nucleotide sequence represented by SEQ ID NO: 77893.

The probes having the nucleotide sequence represented by SEQ ID NOS: 111324, 111325, 111326, 111327, 111328, 111329, 111330, 111331, 111332, 111333, 111334 were selected from the ORFs having the nucleotide sequence represented by SEQ ID NO: 77894.

The probes having the nucleotide sequence represented by SEQ ID NOS: 111335, 111336, 111337, 111338, 111339, 111340, 111341, 111342, 111343, 111344, 111345 were selected from the ORFs having the nucleotide sequence represented by SEQ ID NO: 77895.

The probes having the nucleotide sequence represented by SEQ ID NOS: 111346, 111347, 111348, 111349, 111350, 111351, 111352, 111353, 111354, 111355, 111356 were selected from the ORFs having the nucleotide sequence represented by SEQ ID NO: 77896.

The probes having the nucleotide sequence represented by SEQ ID NOS: 111357, 111358, 111359, 111360, 111361, 111362, 111363, 111364, 111365, 111366, 111367 were selected from the ORFs having the nucleotide sequence represented by SEQ ID NO: 77897.

The probes having the nucleotide sequence represented by SEQ ID NOS: 111368, 111369, 111370, 111371, 111372, 111373, 111374, 111375, 111376, 111377, 111378 were selected from the ORFs having the nucleotide sequence represented by SEQ ID NO: 77898.

The probes having the nucleotide sequence represented by SEQ ID NOS: 111379, 111380, 111381, 111382, 111383, 111384, 111385, 111386, 111387, 111388, 111389 were selected from the ORFs having the nucleotide sequence represented by SEQ ID NO: 77899.

The probes having the nucleotide sequence represented by SEQ ID NOS: 111390, 111391, 111392, 111393, 111394, 111395, 111396, 111397, 111398, 111399, 111400 were selected from the ORFs having the nucleotide sequence represented by SEQ ID NO: 77900.

The probes having the nucleotide sequence represented by SEQ ID NOS: 111401, 111402, 111403, 111404, 111405, 111406, 111407, 111408, 111409, 111410, 111411 were selected from the ORFs having the nucleotide sequence represented by SEQ ID NO: 77901.

The probes having the nucleotide sequence represented by SEQ ID NOS: 111412, 111413, 111414, 111415, 111416, 111417, 111418, 111419, 111420, 111421, 111422 were selected from the ORFs having the nucleotide sequence represented by SEQ ID NO: 77902.

The probes having the nucleotide sequence represented by SEQ ID NOS: 111423, 111424, 111425, 111426, 111427, 111428, 111429, 111430, 111431, 111432, 111433 were selected from the ORFs having the nucleotide sequence represented by SEQ ID NO: 77903.

The probes having the nucleotide sequence represented by SEQ ID NOS: 111434, 111435, 111436, 111437, 111438, 111439, 111440, 111441, 111442, 111443, 111444 were selected from the ORFs having the nucleotide sequence represented by SEQ ID NO: 77904.

The probes having the nucleotide sequence represented by SEQ ID NOS: 111445, 111446, 111447, 111448, 111449, 111450, 111451, 111452, 111453, 111454, 111455 were selected from the ORFs having the nucleotide sequence represented by SEQ ID NO: 77905.

The probes having the nucleotide sequence represented by SEQ ID NOS: 111456, 111457, 111458, 111459, 111460, 111461, 111462, 111463, 111464, 111465, 111466 were selected from the ORFs having the nucleotide sequence represented by SEQ ID NO: 77906.

The probes having the nucleotide sequence represented by SEQ ID NOS: 111467, 111468, 111469, 111470, 111471, 111472, 111473, 111474, 111475, 111476, 111477 were selected from the ORFs having the nucleotide sequence represented by SEQ ID NO: 77907.

The probes having the nucleotide sequence represented by SEQ ID NOS: 111478, 111479, 111480, 111481, 111482, 111483, 111484, 111485, 111486, 111487, 111488 were selected from the ORFs having the nucleotide sequence represented by SEQ ID NO: 77908.

The probes having the nucleotide sequence represented by SEQ ID NOS: 111489, 111490, 111491, 111492, 111493, 111494, 111495, 111496, 111497, 111498, 111499 were selected from the ORFs having the nucleotide sequence represented by SEQ ID NO: 77909.

The probes having the nucleotide sequence represented by SEQ ID NOS: 111500, 111501, 111502, 111503, 111504, 111505, 111506, 111507, 111508, 111509, 111510 were selected from the ORFs having the nucleotide sequence represented by SEQ ID NO: 77910.

The probes having the nucleotide sequence represented by SEQ ID NOS: 111511, 111512, 111513, 111514, 111515, 111516, 111517, 111518, 111519, 111520, 111521 were selected from the ORFs having the nucleotide sequence represented by SEQ ID NO: 77911.

The probes having the nucleotide sequence represented by SEQ ID NOS: 111522, 111523, 111524, 111525, 111526, 111527, 111528, 111529, 111530, 111531, 111532 were selected from the ORFs having the nucleotide sequence represented by SEQ ID NO: 77912.

The probes having the nucleotide sequence represented by SEQ ID NOS: 111533, 111534, 111535, 111536, 111537, 111538, 111539, 111540, 111541, 111542, 111543 were selected from the ORFs having the nucleotide sequence represented by SEQ ID NO: 77913.

The probes having the nucleotide sequence represented by SEQ ID NOS: 111544, 111545, 111546, 111547, 111548, 111549, 111550, 111551, 111552, 111553, 111554 were selected from the ORFs having the nucleotide sequence represented by SEQ ID NO: 77914.

The probes having the nucleotide sequence represented by SEQ ID NOS: 111555, 111556, 111557, 111558, 111559, 111560, 111561, 111562, 111563, 111564, 111565 were selected from the ORFs having the nucleotide sequence represented by SEQ ID NO: 77915.

The probes having the nucleotide sequence represented by SEQ ID NOS: 111566, 111567, 111568, 111569, 111570, 111571, 111572, 111573, 111574, 111575, 111576 were selected from the ORFs having the nucleotide sequence represented by SEQ ID NO: 77916.

The probes having the nucleotide sequence represented by SEQ ID NOS: 111577, 111578, 111579, 111580, 111581, 111582, 111583, 111584, 111585, 111586, 111587 were selected from the ORFs having the nucleotide sequence represented by SEQ ID NO: 77917.

The probes having the nucleotide sequence represented by SEQ ID NOS: 111588, 111589, 111590, 111591, 111592, 111593, 111594, 111595, 111596, 111597, 111598 were selected from the ORFs having the nucleotide sequence represented by SEQ ID NO: 77918.

The probes having the nucleotide sequence represented by SEQ ID NOS: 111599, 111600, 111601, 111602, 111603, 111604, 111605, 111606, 111607, 111608, 111609 were selected from the ORFs having the nucleotide sequence represented by SEQ ID NO: 77919.

The probes having the nucleotide sequence represented by SEQ ID NOS: 111610, 111611, 111612, 111613, 111614, 111615, 111616, 111617, 111618, 111619, 111620 were selected from the ORFs having the nucleotide sequence represented by SEQ ID NO: 77920.

The probes having the nucleotide sequence represented by SEQ ID NOS: 111621, 111622, 111623, 111624, 111625, 111626, 111627, 111628, 111629, 111630, 111631 were selected from the ORFs having the nucleotide sequence represented by SEQ ID NO: 77921.

The probes having the nucleotide sequence represented by SEQ ID NOS: 111632, 111633, 111634, 111635, 111636, 111637, 111638, 111639, 111640, 111641, 111642 were selected from the ORFs having the nucleotide sequence represented by SEQ ID NO: 77922.

The probes having the nucleotide sequence represented by SEQ ID NOS: 111643, 111644, 111645, 111646, 111647, 111648, 111649, 111650, 111651, 111652, 111653 were selected from the ORFs having the nucleotide sequence represented by SEQ ID NO: 77923.

The probes having the nucleotide sequence represented by SEQ ID NOS: 111654, 111655, 111656, 111657, 111658, 111659, 111660, 111661, 111662, 111663, 111664 were selected from the ORFs having the nucleotide sequence represented by SEQ ID NO: 77924.

The probes having the nucleotide sequence represented by SEQ ID NOS: 111665, 111666, 111667, 111668, 111669, 111670, 111671, 111672, 111673, 111674, 111675 were selected from the ORFs having the nucleotide sequence represented by SEQ ID NO: 77925.

The probes having the nucleotide sequence represented by SEQ ID NOS: 111676, 111677, 111678, 111679, 111680, 111681, 111682, 111683, 111684, 111685, 111686 were selected from the ORFs having the nucleotide sequence represented by SEQ ID NO: 77926.

The probes having the nucleotide sequence represented by SEQ ID NOS: 111687, 111688, 111689, 111690, 111691, 111692, 111693, 111694, 111695, 111696, 111697 were selected from the ORFs having the nucleotide sequence represented by SEQ ID NO: 77927.

The probes having the nucleotide sequence represented by SEQ ID NOS: 111698, 111699, 111700, 111701, 111702, 111703, 111704, 111705, 111706, 111707, 111708 were selected from the ORFs having the nucleotide sequence represented by SEQ ID NO: 77928.

The probes having the nucleotide sequence represented by SEQ ID NOS: 111709, 111710, 111711, 111712, 111713, 111714, 111715, 111716, 111717, 111718, 111719 were selected from the ORFs having the nucleotide sequence represented by SEQ ID NO: 77929.

The probes having the nucleotide sequence represented by SEQ ID NOS: 111720, 111721, 111722, 111723, 111724, 111725, 111726, 111727, 111728, 111729, 111730 were selected from the ORFs having the nucleotide sequence represented by SEQ ID NO: 77930.

The probes having the nucleotide sequence represented by SEQ ID NOS: 111731, 111732, 111733, 111734, 111735, 111736, 111737, 111738, 111739, 111740, 111741 were selected from the ORFs having the nucleotide sequence represented by SEQ ID NO: 77931.

The probes having the nucleotide sequence represented by SEQ ID NOS: 111742, 111743, 111744, 111745, 111746, 111747, 111748, 111749, 111750, 111751, 111752 were selected from the ORFs having the nucleotide sequence represented by SEQ ID NO: 77932.

The probes having the nucleotide sequence represented by SEQ ID NOS: 111753, 111754, 111755, 111756, 111757, 111758, 111759, 111760, 111761, 111762, 111763 were selected from the ORFs having the nucleotide sequence represented by SEQ ID NO: 77933.

The probes having the nucleotide sequence represented by SEQ ID NOS: 111764, 111765, 111766, 111767, 111768, 111769, 111770, 111771, 111772, 111773, 111774 were selected from the ORFs having the nucleotide sequence represented by SEQ ID NO: 77934.

The probes having the nucleotide sequence represented by SEQ ID NOS: 111775, 111776, 111777, 111778, 111779, 111780, 111781, 111782, 111783, 111784, 111785 were selected from the ORFs having the nucleotide sequence represented by SEQ ID NO: 77935.

The probes having the nucleotide sequence represented by SEQ ID NOS: 111786, 111787, 111788, 111789, 111790, 111791, 111792, 111793, 111794, 111795, 111796 were selected from the ORFs having the nucleotide sequence represented by SEQ ID NO: 77936.

The probes having the nucleotide sequence represented by SEQ ID NOS: 111797, 111798, 111799, 111800, 111801, 111802, 111803, 111804, 111805, 111806, 111807 were selected from the ORFs having the nucleotide sequence represented by SEQ ID NO: 77937.

The probes having the nucleotide sequence represented by SEQ ID NOS: 111808, 111809, 111810, 111811, 111812, 111813, 111814, 111815, 111816, 111817, 111818 were selected from the ORFs having the nucleotide sequence represented by SEQ ID NO: 77938.

The probes having the nucleotide sequence represented by SEQ ID NOS: 111819, 111820, 111821, 111822, 111823, 111824, 111825, 111826, 111827, 111828, 111829 were selected from the ORFs having the nucleotide sequence represented by SEQ ID NO: 77939.

The probes having the nucleotide sequence represented by SEQ ID NOS: 111830, 111831, 111832, 111833, 111834, 111835, 111836, 111837, 111838, 111839, 111840 were selected from the ORFs having the nucleotide sequence represented by SEQ ID NO: 77940.

The probes having the nucleotide sequence represented by SEQ ID NOS: 111841, 111842, 111843, 111844, 111845, 111846, 111847, 111848, 111849, 111850, 111851 were selected from the ORFs having the nucleotide sequence represented by SEQ ID NO: 77941.

The probes having the nucleotide sequence represented by SEQ ID NOS: 111852, 111853, 111854, 111855, 111856, 111857, 111858, 111859, 111860, 111861, 111862 were selected from the ORFs having the nucleotide sequence represented by SEQ ID NO: 77942.

The probes having the nucleotide sequence represented by SEQ ID NOS: 111863, 111864, 111865, 111866, 111867, 111868, 111869, 111870, 111871, 111872, 111873 were selected from the ORFs having the nucleotide sequence represented by SEQ ID NO: 77943.

The probes having the nucleotide sequence represented by SEQ ID NOS: 111874, 111875, 111876, 111877, 111878, 111879, 111880, 111881, 111882, 111883, 111884 were selected from the ORFs having the nucleotide sequence represented by SEQ ID NO: 77944.

The probes having the nucleotide sequence represented by SEQ ID NOS: 111885, 111886, 111887, 111888, 111889, 111890, 111891, 111892, 111893, 111894, 111895 were selected from the ORFs having the nucleotide sequence represented by SEQ ID NO: 77945.

The probes having the nucleotide sequence represented by SEQ ID NOS: 111896, 111897, 111898, 111899, 111900, 111901, 111902, 111903, 111904, 111905, 111906 were selected from the ORFs having the nucleotide sequence represented by SEQ ID NO: 77946.

The probes having the nucleotide sequence represented by SEQ ID NOS: 111907, 111908, 111909, 111910, 111911, 111912, 111913, 111914, 111915, 111916, 111917 were selected from the ORFs having the nucleotide sequence represented by SEQ ID NO: 77947.

The probes having the nucleotide sequence represented by SEQ ID NOS: 111918, 111919, 111920, 111921, 111922, 111923, 111924, 111925, 111926, 111927, 111928 were selected from the ORFs having the nucleotide sequence represented by SEQ ID NO: 77948.

The probes having the nucleotide sequence represented by SEQ ID NOS: 111929, 111930, 111931, 111932, 111933, 111934, 111935, 111936, 111937, 111938, 111939 were selected from the ORFs having the nucleotide sequence represented by SEQ ID NO: 77949.

The probes having the nucleotide sequence represented by SEQ ID NOS: 111940, 111941, 111942, 111943, 111944, 111945, 111946, 111947, 111948, 111949, 111950 were selected from the ORFs having the nucleotide sequence represented by SEQ ID NO: 77950.

The probes having the nucleotide sequence represented by SEQ ID NOS: 111951, 111952, 111953, 111954, 111955, 111956, 111957, 111958, 111959, 111960, 111961 were selected from the ORFs having the nucleotide sequence represented by SEQ ID NO: 77951.

The probes having the nucleotide sequence represented by SEQ ID NOS: 111962, 111963, 111964, 111965, 111966, 111967, 111968, 111969, 111970, 111971, 111972 were selected from the ORFs having the nucleotide sequence represented by SEQ ID NO: 77952.

The probes having the nucleotide sequence represented by SEQ ID NOS: 111973, 111974, 111975, 111976, 111977, 111978, 111979, 111980, 111981, 111982, 111983 were selected from the ORFs having the nucleotide sequence represented by SEQ ID NO: 77953.

The probes having the nucleotide sequence represented by SEQ ID NOS: 111984, 111985, 111986, 111987, 111988, 111989, 111990, 111991, 111992, 111993, 111994 were selected from the ORFs having the nucleotide sequence represented by SEQ ID NO: 77954.

The probes having the nucleotide sequence represented by SEQ ID NOS: 111995, 111996, 111997, 111998, 111999, 112000, 112001, 112002, 112003, 112004, 112005 were selected from the ORFs having the nucleotide sequence represented by SEQ ID NO: 77955.

The probes having the nucleotide sequence represented by SEQ ID NOS: 112006, 112007, 112008, 112009, 112010, 112011, 112012, 112013, 112014, 112015, 112016 were selected from the ORFs having the nucleotide sequence represented by SEQ ID NO: 77956.

The probes having the nucleotide sequence represented by SEQ ID NOS: 112017, 112018, 112019, 112020, 112021, 112022, 112023, 112024, 112025, 112026, 112027 were selected from the ORFs having the nucleotide sequence represented by SEQ ID NO: 77957.

The probes having the nucleotide sequence represented by SEQ ID NOS: 112028, 112029, 112030, 112031, 112032, 112033, 112034, 112035, 112036, 112037, 112038 were selected from the ORFs having the nucleotide sequence represented by SEQ ID NO: 77958.

The probes having the nucleotide sequence represented by SEQ ID NOS: 112039, 112040, 112041, 112042, 112043, 112044, 112045, 112046, 112047, 112048, 112049 were selected from the ORFs having the nucleotide sequence represented by SEQ ID NO: 77959.

The probes having the nucleotide sequence represented by SEQ ID NOS: 112050, 112051, 112052, 112053, 112054, 112055, 112056, 112057, 112058, 112059, 112060 were selected from the ORFs having the nucleotide sequence represented by SEQ ID NO: 77960.

The probes having the nucleotide sequence represented by SEQ ID NOS: 112061, 112062, 112063, 112064, 112065, 112066, 112067, 112068, 112069, 112070, 112071 were selected from the ORFs having the nucleotide sequence represented by SEQ ID NO: 77961.

The probes having the nucleotide sequence represented by SEQ ID NOS: 112072, 112073, 112074, 112075, 112076, 112077, 112078, 112079, 112080, 112081, 112082 were selected from the ORFs having the nucleotide sequence represented by SEQ ID NO: 77962.

The probes having the nucleotide sequence represented by SEQ ID NOS: 112083, 112084, 112085, 112086, 112087, 112088, 112089, 112090, 112091, 112092, 112093 were selected from the ORFs having the nucleotide sequence represented by SEQ ID NO: 77963.

The probes having the nucleotide sequence represented by SEQ ID NOS: 112094, 112095, 112096, 112097, 112098, 112099, 112100, 112101, 112102, 112103, 112104 were selected from the ORFs having the nucleotide sequence represented by SEQ ID NO: 77964.

The probes having the nucleotide sequence represented by SEQ ID NOS: 112105, 112106, 112107, 112108, 112109, 112110, 112111, 112112, 112113, 112114, 112115 were selected from the ORFs having the nucleotide sequence represented by SEQ ID NO: 77965.

The probes having the nucleotide sequence represented by SEQ ID NOS: 112116, 112117, 112118, 112119, 112120, 112121, 112122, 112123, 112124, 112125, 112126 were selected from the ORFs having the nucleotide sequence represented by SEQ ID NO: 77966.

The probes having the nucleotide sequence represented by SEQ ID NOS: 112127, 112128, 112129, 112130, 112131, 112132, 112133, 112134, 112135, 112136, 112137 were selected from the ORFs having the nucleotide sequence represented by SEQ ID NO: 77967.

The probes having the nucleotide sequence represented by SEQ ID NOS: 112138, 112139, 112140, 112141, 112142, 112143, 112144, 112145, 112146, 112147, 112148 were selected from the ORFs having the nucleotide sequence represented by SEQ ID NO: 77968.

The probes having the nucleotide sequence represented by SEQ ID NOS: 112149, 112150, 112151, 112152, 112153, 112154, 112155, 112156, 112157, 112158, 112159 were selected from the ORFs having the nucleotide sequence represented by SEQ ID NO: 77969.

The probes having the nucleotide sequence represented by SEQ ID NOS: 112160, 112161, 112162, 112163, 112164, 112165, 112166, 112167, 112168, 112169, 112170 were selected from the ORFs having the nucleotide sequence represented by SEQ ID NO: 77970.

The probes having the nucleotide sequence represented by SEQ ID NOS: 112171, 112172, 112173, 112174, 112175, 112176, 112177, 112178, 112179, 112180, 112181 were selected from the ORFs having the nucleotide sequence represented by SEQ ID NO: 77971.

The probes having the nucleotide sequence represented by SEQ ID NOS: 112182, 112183, 112184, 112185, 112186, 112187, 112188, 112189, 112190, 112191, 112192 were selected from the ORFs having the nucleotide sequence represented by SEQ ID NO: 77972.

The probes having the nucleotide sequence represented by SEQ ID NOS: 112193, 112194, 112195, 112196, 112197, 112198, 112199, 112200, 112201, 112202, 112203 were selected from the ORFs having the nucleotide sequence represented by SEQ ID NO: 77973.

The probes having the nucleotide sequence represented by SEQ ID NOS: 112204, 112205, 112206, 112207, 112208, 112209, 112210, 112211, 112212, 112213, 112214 were selected from the ORFs having the nucleotide sequence represented by SEQ ID NO: 77974.

The probes having the nucleotide sequence represented by SEQ ID NOS: 112215, 112216, 112217, 112218, 112219, 112220, 112221, 112222, 112223, 112224, 112225 were selected from the ORFs having the nucleotide sequence represented by SEQ ID NO: 77975.

The probes having the nucleotide sequence represented by SEQ ID NOS: 112226, 112227, 112228, 112229, 112230, 112231, 112232, 112233, 112234, 112235, 112236 were selected from the ORFs having the nucleotide sequence represented by SEQ ID NO: 77976.

The probes having the nucleotide sequence represented by SEQ ID NOS: 112237, 112238, 112239, 112240, 112241, 112242, 112243, 112244, 112245, 112246, 112247 were selected from the ORFs having the nucleotide sequence represented by SEQ ID NO: 77977.

The probes having the nucleotide sequence represented by SEQ ID NOS: 112248, 112249, 112250, 112251, 112252, 112253, 112254, 112255, 112256, 112257, 112258 were selected from the ORFs having the nucleotide sequence represented by SEQ ID NO: 77978.

The probes having the nucleotide sequence represented by SEQ ID NOS: 112259, 112260, 112261, 112262, 112263, 112264, 112265, 112266, 112267, 112268, 112269 were selected from the ORFs having the nucleotide sequence represented by SEQ ID NO: 77979.

The probes having the nucleotide sequence represented by SEQ ID NOS: 112270, 112271, 112272, 112273, 112274, 112275, 112276, 112277, 112278, 112279, 112280 were selected from the ORFs having the nucleotide sequence represented by SEQ ID NO: 77980.

The probes having the nucleotide sequence represented by SEQ ID NOS: 112281, 112282, 112283, 112284, 112285, 112286, 112287, 112288, 112289, 112290, 112291 were selected from the ORFs having the nucleotide sequence represented by SEQ ID NO: 77981.

The probes having the nucleotide sequence represented by SEQ ID NOS: 112292, 112293, 112294, 112295, 112296, 112297, 112298, 112299, 112300, 112301, 112302 were selected from the ORFs having the nucleotide sequence represented by SEQ ID NO: 77982.

The probes having the nucleotide sequence represented by SEQ ID NOS: 112303, 112304, 112305, 112306, 112307, 112308, 112309, 112310, 112311, 112312, 112313 were selected from the ORFs having the nucleotide sequence represented by SEQ ID NO: 77983.

The probes having the nucleotide sequence represented by SEQ ID NOS: 112314, 112315, 112316, 112317, 112318, 112319, 112320, 112321, 112322, 112323, 112324 were selected from the ORFs having the nucleotide sequence represented by SEQ ID NO: 77984.

The probes having the nucleotide sequence represented by SEQ ID NOS: 112325, 112326, 112327, 112328, 112329, 112330, 112331, 112332, 112333, 112334, 112335 were selected from the ORFs having the nucleotide sequence represented by SEQ ID NO: 77985.

The probes having the nucleotide sequence represented by SEQ ID NOS: 112336, 112337, 112338, 112339, 112340, 112341, 112342, 112343, 112344, 112345, 112346 were selected from the ORFs having the nucleotide sequence represented by SEQ ID NO: 77986.

The probes having the nucleotide sequence represented by SEQ ID NOS: 112347, 112348, 112349, 112350, 112351, 112352, 112353, 112354, 112355, 112356, 112357 were selected from the ORFs having the nucleotide sequence represented by SEQ ID NO: 77987.

The probes having the nucleotide sequence represented by SEQ ID NOS: 112358, 112359, 112360, 112361, 112362, 112363, 112364, 112365, 112366, 112367, 112368 were selected from the ORFs having the nucleotide sequence represented by SEQ ID NO: 77988.

The probes having the nucleotide sequence represented by SEQ ID NOS: 112369, 112370, 112371, 112372, 112373, 112374, 112375, 112376, 112377, 112378, 112379 were selected from the ORFs having the nucleotide sequence represented by SEQ ID NO: 77989.

The probes having the nucleotide sequence represented by SEQ ID NOS: 112380, 112381, 112382, 112383, 112384, 112385, 112386, 112387, 112388, 112389, 112390 were selected from the ORFs having the nucleotide sequence represented by SEQ ID NO: 77990.

The probes having the nucleotide sequence represented by SEQ ID NOS: 112391, 112392, 112393, 112394, 112395, 112396, 112397, 112398, 112399, 112400, 112401 were selected from the ORFs having the nucleotide sequence represented by SEQ ID NO: 77991.

The probes having the nucleotide sequence represented by SEQ ID NOS: 112402, 112403, 112404, 112405, 112406, 112407, 112408, 112409, 112410, 112411, 112412 were selected from the ORFs having the nucleotide sequence represented by SEQ ID NO: 77992.

The probes having the nucleotide sequence represented by SEQ ID NOS: 112413, 112414, 112415, 112416, 112417, 112418, 112419, 112420, 112421, 112422, 112423 were selected from the ORFs having the nucleotide sequence represented by SEQ ID NO: 77993.

The probes having the nucleotide sequence represented by SEQ ID NOS: 112424, 112425, 112426, 112427, 112428, 112429, 112430, 112431, 112432, 112433, 112434 were selected from the ORFs having the nucleotide sequence represented by SEQ ID NO: 77994.

The probes having the nucleotide sequence represented by SEQ ID NOS: 112435, 112436, 112437, 112438, 112439, 112440, 112441, 112442, 112443, 112444, 112445, 112446, 112447, 112448, 112449, 112450, 112451, 112452, 112453, 112454, 112455, 112456 were selected from the ORFs having the nucleotide sequence represented by SEQ ID NO: 77995.

The probes having the nucleotide sequence represented by SEQ ID NOS: 112457, 112458, 112459, 112460, 112461, 112462, 112463, 112464, 112465, 112466, 112467 were selected from the ORFs having the nucleotide sequence represented by SEQ ID NO: 77996.

The probes having the nucleotide sequence represented by SEQ ID NOS: 112468, 112469, 112470, 112471, 112472, 112473, 112474, 112475, 112476, 112477, 112478 were selected from the ORFs having the nucleotide sequence represented by SEQ ID NO: 77997.

The probes having the nucleotide sequence represented by SEQ ID NOS: 112479, 112480, 112481, 112482, 112483, 112484, 112485, 112486, 112487, 112488, 112489 were selected from the ORFs having the nucleotide sequence represented by SEQ ID NO: 77998.

The probes having the nucleotide sequence represented by SEQ ID NOS: 112490, 112491, 112492, 112493, 112494, 112495, 112496, 112497, 112498, 112499, 112500 were selected from the ORFs having the nucleotide sequence represented by SEQ ID NO: 77999.

The probes having the nucleotide sequence represented by SEQ ID NOS: 112501, 112502, 112503, 112504, 112505, 112506, 112507, 112508, 112509, 112510, 112511 were selected from the ORFs having the nucleotide sequence represented by SEQ ID NO: 78000.

The probes having the nucleotide sequence represented by SEQ ID NOS: 112512, 112513, 112514, 112515, 112516, 112517, 112518, 112519, 112520, 112521, 112522 were selected from the ORFs having the nucleotide sequence represented by SEQ ID NO: 78001.

The probes having the nucleotide sequence represented by SEQ ID NOS: 112523, 112524, 112525, 112526, 112527, 112528, 112529, 112530, 112531, 112532, 112533 were selected from the ORFs having the nucleotide sequence represented by SEQ ID NO: 78002.

The probes having the nucleotide sequence represented by SEQ ID NOS: 112534, 112535, 112536, 112537, 112538, 112539, 112540, 112541, 112542, 112543, 112544 were selected from the ORFs having the nucleotide sequence represented by SEQ ID NO: 78003.

The probes having the nucleotide sequence represented by SEQ ID NOS: 112545, 112546, 112547, 112548, 112549, 112550, 112551, 112552, 112553, 112554, 112555 were selected from the ORFs having the nucleotide sequence represented by SEQ ID NO: 78004.

The probes having the nucleotide sequence represented by SEQ ID NOS: 112556, 112557, 112558, 112559, 112560, 112561, 112562, 112563, 112564, 112565, 112566, 112567, 112568, 112569, 112570, 112571, 112572, 112573, 112574, 112575, 112576, 112577 were selected from the ORFs having the nucleotide sequence represented by SEQ ID NO: 78005.

The probes having the nucleotide sequence represented by SEQ ID NOS: 112578, 112579, 112580, 112581, 112582, 112583, 112584, 112585, 112586, 112587, 112588 were selected from the ORFs having the nucleotide sequence represented by SEQ ID NO: 78006.

The probes having the nucleotide sequence represented by SEQ ID NOS: 112589, 112590, 112591, 112592, 112593, 112594, 112595, 112596, 112597, 112598, 112599 were selected from the ORFs having the nucleotide sequence represented by SEQ ID NO: 78007.

The probes having the nucleotide sequence represented by SEQ ID NOS: 112600, 112601, 112602, 112603, 112604, 112605, 112606, 112607, 112608, 112609, 112610 were selected from the ORFs having the nucleotide sequence represented by SEQ ID NO: 78008.

The probes having the nucleotide sequence represented by SEQ ID NOS: 112611, 112612, 112613, 112614, 112615, 112616, 112617, 112618, 112619, 112620, 112621 were selected from the ORFs having the nucleotide sequence represented by SEQ ID NO: 78009.

The probes having the nucleotide sequence represented by SEQ ID NOS: 112622, 112623, 112624, 112625, 112626, 112627, 112628, 112629, 112630, 112631, 112632 were selected from the ORFs having the nucleotide sequence represented by SEQ ID NO: 78010.

The probes having the nucleotide sequence represented by SEQ ID NOS: 112633, 112634, 112635, 112636, 112637, 112638, 112639, 112640, 112641, 112642, 112643 were selected from the ORFs having the nucleotide sequence represented by SEQ ID NO: 78011.

The probes having the nucleotide sequence represented by SEQ ID NOS: 112644, 112645, 112646, 112647, 112648, 112649, 112650, 112651, 112652, 112653, 112654 were selected from the ORFs having the nucleotide sequence represented by SEQ ID NO: 78012.

The probes having the nucleotide sequence represented by SEQ ID NOS: 112655, 112656, 112657, 112658, 112659, 112660, 112661, 112662, 112663, 112664, 112665 were selected from the ORFs having the nucleotide sequence represented by SEQ ID NO: 78013.

The probes having the nucleotide sequence represented by SEQ ID NOS: 112666, 112667, 112668, 112669, 112670, 112671, 112672, 112673, 112674, 112675, 112676 were selected from the ORFs having the nucleotide sequence represented by SEQ ID NO: 78014.

The probes having the nucleotide sequence represented by SEQ ID NOS: 112677, 112678, 112679, 112680, 112681, 112682, 112683, 112684, 112685, 112686, 112687 were selected from the ORFs having the nucleotide sequence represented by SEQ ID NO: 78015.

The probes having the nucleotide sequence represented by SEQ ID NOS: 112688, 112689, 112690, 112691, 112692, 112693, 112694, 112695, 112696, 112697, 112698 were selected from the ORFs having the nucleotide sequence represented by SEQ ID NO: 78016.

The probes having the nucleotide sequence represented by SEQ ID NOS: 112699, 112700, 112701, 112702, 112703, 112704, 112705, 112706, 112707, 112708, 112709 were selected from the ORFs having the nucleotide sequence represented by SEQ ID NO: 78017.

The probes having the nucleotide sequence represented by SEQ ID NOS: 112710, 112711, 112712, 112713, 112714, 112715, 112716, 112717, 112718, 112719, 112720 were selected from the ORFs having the nucleotide sequence represented by SEQ ID NO: 78018.

The probes having the nucleotide sequence represented by SEQ ID NOS: 112721, 112722, 112723, 112724, 112725, 112726, 112727, 112728, 112729, 112730, 112731 were selected from the ORFs having the nucleotide sequence represented by SEQ ID NO: 78019.

The probes having the nucleotide sequence represented by SEQ ID NOS: 112732, 112733, 112734, 112735, 112736, 112737, 112738, 112739, 112740, 112741, 112742 were selected from the ORFs having the nucleotide sequence represented by SEQ ID NO: 78020.

The probes having the nucleotide sequence represented by SEQ ID NOS: 112743, 112744, 112745, 112746, 112747, 112748, 112749, 112750, 112751, 112752, 112753 were selected from the ORFs having the nucleotide sequence represented by SEQ ID NO: 78021.

The probes having the nucleotide sequence represented by SEQ ID NOS: 112754, 112755, 112756, 112757, 112758, 112759, 112760, 112761, 112762, 112763, 112764 were selected from the ORFs having the nucleotide sequence represented by SEQ ID NO: 78022.

The probes having the nucleotide sequence represented by SEQ ID NOS: 112765, 112766, 112767, 112768, 112769, 112770, 112771, 112772, 112773, 112774, 112775 were selected from the ORFs having the nucleotide sequence represented by SEQ ID NO: 78023.

The probes having the nucleotide sequence represented by SEQ ID NOS: 112776, 112777, 112778, 112779, 112780, 112781, 112782, 112783, 112784, 112785, 112786 were selected from the ORFs having the nucleotide sequence represented by SEQ ID NO: 78024.

The probes having the nucleotide sequence represented by SEQ ID NOS: 112787, 112788, 112789, 112790, 112791, 112792, 112793, 112794, 112795, 112796, 112797 were selected from the ORFs having the nucleotide sequence represented by SEQ ID NO: 78025.

The probes having the nucleotide sequence represented by SEQ ID NOS: 112798, 112799, 112800, 112801, 112802, 112803, 112804, 112805, 112806, 112807, 112808 were selected from the ORFs having the nucleotide sequence represented by SEQ ID NO: 78026.

The probes having the nucleotide sequence represented by SEQ ID NOS: 112809, 112810, 112811, 112812, 112813, 112814, 112815, 112816, 112817, 112818, 112819 were selected from the ORFs having the nucleotide sequence represented by SEQ ID NO: 78027.

The probes having the nucleotide sequence represented by SEQ ID NOS: 112820, 112821, 112822, 112823, 112824, 112825, 112826, 112827, 112828, 112829, 112830 were selected from the ORFs having the nucleotide sequence represented by SEQ ID NO: 78028.

The probes having the nucleotide sequence represented by SEQ ID NOS: 112831, 112832, 112833, 112834, 112835, 112836, 112837, 112838, 112839, 112840, 112841 were selected from the ORFs having the nucleotide sequence represented by SEQ ID NO: 78029.

The probes having the nucleotide sequence represented by SEQ ID NOS: 112842, 112843, 112844, 112845, 112846, 112847, 112848, 112849, 112850, 112851, 112852 were selected from the ORFs having the nucleotide sequence represented by SEQ ID NO: 78030.

The probes having the nucleotide sequence represented by SEQ ID NOS: 112853, 112854, 112855, 112856, 112857, 112858, 112859, 112860, 112861, 112862, 112863 were selected from the ORFs having the nucleotide sequence represented by SEQ ID NO: 78031.

The probes having the nucleotide sequence represented by SEQ ID NOS: 112864, 112865, 112866, 112867, 112868, 112869, 112870, 112871, 112872, 112873, 112874 were selected from the ORFs having the nucleotide sequence represented by SEQ ID NO: 78032.

The probes having the nucleotide sequence represented by SEQ ID NOS: 112875, 112876, 112877, 112878, 112879, 112880, 112881, 112882, 112883, 112884, 112885 were selected from the ORFs having the nucleotide sequence represented by SEQ ID NO: 78033.

The probes having the nucleotide sequence represented by SEQ ID NOS: 112886, 112887, 112888, 112889, 112890, 112891, 112892, 112893, 112894, 112895, 112896 were selected from the ORFs having the nucleotide sequence represented by SEQ ID NO: 78034.

The probes having the nucleotide sequence represented by SEQ ID NOS: 112897, 112898, 112899, 112900, 112901, 112902, 112903, 112904, 112905, 112906, 112907 were selected from the ORFs having the nucleotide sequence represented by SEQ ID NO: 78035.

The probes having the nucleotide sequence represented by SEQ ID NOS: 112908, 112909, 112910, 112911, 112912, 112913, 112914, 112915, 112916, 112917, 112918 were selected from the ORFs having the nucleotide sequence represented by SEQ ID NO: 78036.

The probes having the nucleotide sequence represented by SEQ ID NOS: 112919, 112920, 112921, 112922, 112923, 112924, 112925, 112926, 112927, 112928, 112929 were selected from the ORFs having the nucleotide sequence represented by SEQ ID NO: 78037.

The probes having the nucleotide sequence represented by SEQ ID NOS: 112930, 112931, 112932, 112933, 112934, 112935, 112936, 112937, 112938, 112939, 112940 were selected from the ORFs having the nucleotide sequence represented by SEQ ID NO: 78038.

The probes having the nucleotide sequence represented by SEQ ID NOS: 112941, 112942, 112943, 112944, 112945, 112946, 112947, 112948, 112949, 112950, 112951 were selected from the ORFs having the nucleotide sequence represented by SEQ ID NO: 78039.

The probes having the nucleotide sequence represented by SEQ ID NOS: 112952, 112953, 112954, 112955, 112956, 112957, 112958, 112959, 112960, 112961, 112962 were selected from the ORFs having the nucleotide sequence represented by SEQ ID NO: 78040.

The probes having the nucleotide sequence represented by SEQ ID NOS: 112963, 112964, 112965, 112966, 112967, 112968, 112969, 112970, 112971, 112972, 112973 were selected from the ORFs having the nucleotide sequence represented by SEQ ID NO: 78041.

The probes having the nucleotide sequence represented by SEQ ID NOS: 112974, 112975, 112976, 112977, 112978, 112979, 112980, 112981, 112982, 112983, 112984 were selected from the ORFs having the nucleotide sequence represented by SEQ ID NO: 78042.

The probes having the nucleotide sequence represented by SEQ ID NOS: 112985, 112986, 112987, 112988, 112989, 112990, 112991, 112992, 112993, 112994, 112995 were selected from the ORFs having the nucleotide sequence represented by SEQ ID NO: 78043.

The probes having the nucleotide sequence represented by SEQ ID NOS: 112996, 112997, 112998, 112999, 113000, 113001, 113002, 113003, 113004, 113005, 113006 were selected from the ORFs having the nucleotide sequence represented by SEQ ID NO: 78044.

The probes having the nucleotide sequence represented by SEQ ID NOS: 113007, 113008, 113009, 113010, 113011, 113012, 113013, 113014, 113015, 113016, 113017, 113018, 113019, 113020, 113021, 113022, 113023, 113024, 113025, 113026, 113027, 113028 were selected from the ORFs having the nucleotide sequence represented by SEQ ID NO: 78045.

The probes having the nucleotide sequence represented by SEQ ID NOS: 113029, 113030, 113031, 113032, 113033, 113034, 113035, 113036, 113037, 113038, 113039 were selected from the ORFs having the nucleotide sequence represented by SEQ ID NO: 78046.

The probes having the nucleotide sequence represented by SEQ ID NOS: 113040, 113041, 113042, 113043, 113044, 113045, 113046, 113047, 113048, 113049, 113050 were selected from the ORFs having the nucleotide sequence represented by SEQ ID NO: 78047.

The probes having the nucleotide sequence represented by SEQ ID NOS: 113051, 113052, 113053, 113054, 113055, 113056, 113057, 113058, 113059, 113060, 113061 were selected from the ORFs having the nucleotide sequence represented by SEQ ID NO: 78048.

The probes having the nucleotide sequence represented by SEQ ID NOS: 113062, 113063, 113064, 113065, 113066, 113067, 113068, 113069, 113070, 113071, 113072 were selected from the ORFs having the nucleotide sequence represented by SEQ ID NO: 78049.

The probes having the nucleotide sequence represented by SEQ ID NOS: 113073, 113074, 113075, 113076, 113077, 113078, 113079, 113080, 113081, 113082, 113083 were selected from the ORFs having the nucleotide sequence represented by SEQ ID NO: 78050.

The probes having the nucleotide sequence represented by SEQ ID NOS: 113084, 113085, 113086, 113087, 113088, 113089, 113090, 113091, 113092, 113093, 113094 were selected from the ORFs having the nucleotide sequence represented by SEQ ID NO: 78051.

The probes having the nucleotide sequence represented by SEQ ID NOS: 113095, 113096, 113097, 113098, 113099, 113100, 113101, 113102, 113103, 113104, 113105 were selected from the ORFs having the nucleotide sequence represented by SEQ ID NO: 78052.

The probes having the nucleotide sequence represented by SEQ ID NOS: 113106, 113107, 113108, 113109, 113110, 113111, 113112, 113113, 113114, 113115, 113116 were selected from the ORFs having the nucleotide sequence represented by SEQ ID NO: 78053.

The probes having the nucleotide sequence represented by SEQ ID NOS: 113117, 113118, 113119, 113120, 113121, 113122, 113123, 113124, 113125, 113126, 113127 were selected from the ORFs having the nucleotide sequence represented by SEQ ID NO: 78054.

The probes having the nucleotide sequence represented by SEQ ID NOS: 113128, 113129, 113130, 113131, 113132, 113133, 113134, 113135, 113136, 113137, 113138 were selected from the ORFs having the nucleotide sequence represented by SEQ ID NO: 78055.

The probes having the nucleotide sequence represented by SEQ ID NOS: 113139, 113140, 113141, 113142, 113143, 113144, 113145, 113146, 113147, 113148, 113149 were selected from the ORFs having the nucleotide sequence represented by SEQ ID NO: 78056.

The probes having the nucleotide sequence represented by SEQ ID NOS: 113150, 113151, 113152, 113153, 113154, 113155, 113156, 113157, 113158, 113159, 113160 were selected from the ORFs having the nucleotide sequence represented by SEQ ID NO: 78057.

The probes having the nucleotide sequence represented by SEQ ID NOS: 113161, 113162, 113163, 113164, 113165, 113166, 113167, 113168, 113169, 113170, 113171 were selected from the ORFs having the nucleotide sequence represented by SEQ ID NO: 78058.

The probes having the nucleotide sequence represented by SEQ ID NOS: 113172, 113173, 113174, 113175, 113176, 113177, 113178, 113179, 113180, 113181, 113182 were selected from the ORFs having the nucleotide sequence represented by SEQ ID NO: 78059.

The probes having the nucleotide sequence represented by SEQ ID NOS: 113183, 113184, 113185, 113186, 113187, 113188, 113189, 113190, 113191, 113192, 113193 were selected from the ORFs having the nucleotide sequence represented by SEQ ID NO: 78060.

The probes having the nucleotide sequence represented by SEQ ID NOS: 113194, 113195, 113196, 113197, 113198, 113199, 113200, 113201, 113202, 113203, 113204 were selected from the ORFs having the nucleotide sequence represented by SEQ ID NO: 78061.

The probes having the nucleotide sequence represented by SEQ ID NOS: 113205, 113206, 113207, 113208, 113209, 113210, 113211, 113212, 113213, 113214, 113215 were selected from the ORFs having the nucleotide sequence represented by SEQ ID NO: 78062.

The probes having the nucleotide sequence represented by SEQ ID NOS: 113216, 113217, 113218, 113219, 113220, 113221, 113222, 113223, 113224, 113225, 113226 were selected from the ORFs having the nucleotide sequence represented by SEQ ID NO: 78063.

The probes having the nucleotide sequence represented by SEQ ID NOS: 113227, 113228, 113229, 113230, 113231, 113232, 113233, 113234, 113235, 113236, 113237 were selected from the ORFs having the nucleotide sequence represented by SEQ ID NO: 78064.

The probes having the nucleotide sequence represented by SEQ ID NOS: 113238, 113239, 113240, 113241, 113242, 113243, 113244, 113245, 113246, 113247, 113248 were selected from the ORFs having the nucleotide sequence represented by SEQ ID NO: 78065.

The probes having the nucleotide sequence represented by SEQ ID NOS: 113249, 113250, 113251, 113252, 113253, 113254, 113255, 113256, 113257, 113258, 113259 were selected from the ORFs having the nucleotide sequence represented by SEQ ID NO: 78066.

The probes having the nucleotide sequence represented by SEQ ID NOS: 113260, 113261, 113262, 113263, 113264, 113265, 113266, 113267, 113268, 113269, 113270 were selected from the ORFs having the nucleotide sequence represented by SEQ ID NO: 78067.

The probes having the nucleotide sequence represented by SEQ ID NOS: 113271, 113272, 113273, 113274, 113275, 113276, 113277, 113278, 113279, 113280, 113281 were selected from the ORFs having the nucleotide sequence represented by SEQ ID NO: 78068.

The probes having the nucleotide sequence represented by SEQ ID NOS: 113282, 113283, 113284, 113285, 113286, 113287, 113288, 113289, 113290, 113291, 113292 were selected from the ORFs having the nucleotide sequence represented by SEQ ID NO: 78069.

The probes having the nucleotide sequence represented by SEQ ID NOS: 113293, 113294, 113295, 113296, 113297, 113298, 113299, 113300, 113301, 113302, 113303 were selected from the ORFs having the nucleotide sequence represented by SEQ ID NO: 78070.

The probes having the nucleotide sequence represented by SEQ ID NOS: 113304, 113305, 113306, 113307, 113308, 113309, 113310, 113311, 113312, 113313, 113314 were selected from the ORFs having the nucleotide sequence represented by SEQ ID NO: 78071.

The probes having the nucleotide sequence represented by SEQ ID NOS: 113315, 113316, 113317, 113318, 113319, 113320, 113321, 113322, 113323, 113324, 113325 were selected from the ORFs having the nucleotide sequence represented by SEQ ID NO: 78072.

The probes having the nucleotide sequence represented by SEQ ID NOS: 113326, 113327, 113328, 113329, 113330, 113331, 113332, 113333, 113334, 113335, 113336 were selected from the ORFs having the nucleotide sequence represented by SEQ ID NO: 78073.

The probes having the nucleotide sequence represented by SEQ ID NOS: 113337, 113338, 113339, 113340, 113341, 113342, 113343, 113344, 113345, 113346, 113347 were selected from the ORFs having the nucleotide sequence represented by SEQ ID NO: 78074.

The probes having the nucleotide sequence represented by SEQ ID NOS: 113348, 113349, 113350, 113351, 113352, 113353, 113354, 113355, 113356, 113357, 113358 were selected from the ORFs having the nucleotide sequence represented by SEQ ID NO: 78075.

The probes having the nucleotide sequence represented by SEQ ID NOS: 113359, 113360, 113361, 113362, 113363, 113364, 113365, 113366, 113367, 113368, 113369 were selected from the ORFs having the nucleotide sequence represented by SEQ ID NO: 78076.

The probes having the nucleotide sequence represented by SEQ ID NOS: 113370, 113371, 113372, 113373, 113374, 113375, 113376, 113377, 113378, 113379, 113380 were selected from the ORFs having the nucleotide sequence represented by SEQ ID NO: 78077.

The probes having the nucleotide sequence represented by SEQ ID NOS: 113381, 113382, 113383, 113384, 113385, 113386, 113387, 113388, 113389, 113390, 113391 were selected from the ORFs having the nucleotide sequence represented by SEQ ID NO: 78078.

The probes having the nucleotide sequence represented by SEQ ID NOS: 113392, 113393, 113394, 113395, 113396, 113397, 113398, 113399, 113400, 113401, 113402 were selected from the ORFs having the nucleotide sequence represented by SEQ ID NO: 78079.

The probes having the nucleotide sequence represented by SEQ ID NOS: 113403, 113404, 113405, 113406, 113407, 113408, 113409, 113410, 113411, 113412, 113413 were selected from the ORFs having the nucleotide sequence represented by SEQ ID NO: 78080.

The probes having the nucleotide sequence represented by SEQ ID NOS: 113414, 113415, 113416, 113417, 113418, 113419, 113420, 113421, 113422, 113423, 113424 were selected from the ORFs having the nucleotide sequence represented by SEQ ID NO: 78081.

The probes having the nucleotide sequence represented by SEQ ID NOS: 113425, 113426, 113427, 113428, 113429, 113430, 113431, 113432, 113433, 113434, 113435 were selected from the ORFs having the nucleotide sequence represented by SEQ ID NO: 78082.

The probes having the nucleotide sequence represented by SEQ ID NOS: 113436, 113437, 113438, 113439, 113440, 113441, 113442, 113443, 113444, 113445, 113446 were selected from the ORFs having the nucleotide sequence represented by SEQ ID NO: 78083.

The probes having the nucleotide sequence represented by SEQ ID NOS: 113447, 113448, 113449, 113450, 113451, 113452, 113453, 113454, 113455, 113456, 113457 were selected from the ORFs having the nucleotide sequence represented by SEQ ID NO: 78084.

The probes having the nucleotide sequence represented by SEQ ID NOS: 113458, 113459, 113460, 113461, 113462, 113463, 113464, 113465, 113466, 113467, 113468 were selected from the ORFs having the nucleotide sequence represented by SEQ ID NO: 78085.

The probes having the nucleotide sequence represented by SEQ ID NOS: 113469, 113470, 113471, 113472, 113473, 113474, 113475, 113476, 113477, 113478, 113479 were selected from the ORFs having the nucleotide sequence represented by SEQ ID NO: 78086.

The probes having the nucleotide sequence represented by SEQ ID NOS: 113480, 113481, 113482, 113483, 113484, 113485, 113486, 113487, 113488, 113489, 113490 were selected from the ORFs having the nucleotide sequence represented by SEQ ID NO: 78087.

The probes having the nucleotide sequence represented by SEQ ID NOS: 113491, 113492, 113493, 113494, 113495, 113496, 113497, 113498, 113499, 113500, 113501 were selected from the ORFs having the nucleotide sequence represented by SEQ ID NO: 78088.

The probes having the nucleotide sequence represented by SEQ ID NOS: 113502, 113503, 113504, 113505, 113506, 113507, 113508, 113509, 113510, 113511, 113512 were selected from the ORFs having the nucleotide sequence represented by SEQ ID NO: 78089.

The probes having the nucleotide sequence represented by SEQ ID NOS: 113513, 113514, 113515, 113516, 113517, 113518, 113519, 113520, 113521, 113522, 113523 were selected from the ORFs having the nucleotide sequence represented by SEQ ID NO: 78090.

The probes having the nucleotide sequence represented by SEQ ID NOS: 113524, 113525, 113526, 113527, 113528, 113529, 113530, 113531, 113532, 113533, 113534 were selected from the ORFs having the nucleotide sequence represented by SEQ ID NO: 78091.

The probes having the nucleotide sequence represented by SEQ ID NOS: 113535, 113536, 113537, 113538, 113539, 113540, 113541, 113542, 113543, 113544, 113545 were selected from the ORFs having the nucleotide sequence represented by SEQ ID NO: 78092.

The probes having the nucleotide sequence represented by SEQ ID NOS: 113546, 113547, 113548, 113549, 113550, 113551, 113552, 113553, 113554, 113555, 113556 were selected from the ORFs having the nucleotide sequence represented by SEQ ID NO: 78093.

The probes having the nucleotide sequence represented by SEQ ID NOS: 113557, 113558, 113559, 113560, 113561, 113562, 113563, 113564, 113565, 113566, 113567 were selected from the ORFs having the nucleotide sequence represented by SEQ ID NO: 78094.

The probes having the nucleotide sequence represented by SEQ ID NOS: 113568, 113569, 113570, 113571, 113572, 113573, 113574, 113575, 113576, 113577, 113578 were selected from the ORFs having the nucleotide sequence represented by SEQ ID NO: 78095.

The probes having the nucleotide sequence represented by SEQ ID NOS: 113579, 113580, 113581, 113582, 113583, 113584, 113585, 113586, 113587, 113588, 113589 were selected from the ORFs having the nucleotide sequence represented by SEQ ID NO: 78096.

The probes having the nucleotide sequence represented by SEQ ID NOS: 113590, 113591, 113592, 113593, 113594, 113595, 113596, 113597, 113598, 113599, 113600 were selected from the ORFs having the nucleotide sequence represented by SEQ ID NO: 78097.

The probes having the nucleotide sequence represented by SEQ ID NOS: 113601, 113602, 113603, 113604, 113605, 113606, 113607, 113608, 113609, 113610, 113611 were selected from the ORFs having the nucleotide sequence represented by SEQ ID NO: 78098.

The probes having the nucleotide sequence represented by SEQ ID NOS: 113612, 113613, 113614, 113615, 113616, 113617, 113618, 113619, 113620, 113621, 113622 were selected from the ORFs having the nucleotide sequence represented by SEQ ID NO: 78099.

The probes having the nucleotide sequence represented by SEQ ID NOS: 113623, 113624, 113625, 113626, 113627, 113628, 113629, 113630, 113631, 113632, 113633 were selected from the ORFs having the nucleotide sequence represented by SEQ ID NO: 78100.

The probes having the nucleotide sequence represented by SEQ ID NOS: 113634, 113635, 113636, 113637, 113638, 113639, 113640, 113641, 113642, 113643, 113644 were selected from the ORFs having the nucleotide sequence represented by SEQ ID NO: 78101.

The probes having the nucleotide sequence represented by SEQ ID NOS: 113645, 113646, 113647, 113648, 113649, 113650, 113651, 113652, 113653, 113654, 113655 were selected from the ORFs having the nucleotide sequence represented by SEQ ID NO: 78102.

The probes having the nucleotide sequence represented by SEQ ID NOS: 113656, 113657, 113658, 113659, 113660, 113661, 113662, 113663, 113664, 113665, 113666 were selected from the ORFs having the nucleotide sequence represented by SEQ ID NO: 78103.

The probes having the nucleotide sequence represented by SEQ ID NOS: 113667, 113668, 113669, 113670, 113671, 113672, 113673, 113674, 113675, 113676, 113677 were selected from the ORFs having the nucleotide sequence represented by SEQ ID NO: 78104.

The probes having the nucleotide sequence represented by SEQ ID NOS: 113678, 113679, 113680, 113681, 113682, 113683, 113684, 113685, 113686, 113687, 113688 were selected from the ORFs having the nucleotide sequence represented by SEQ ID NO: 78105.

The probes having the nucleotide sequence represented by SEQ ID NOS: 113689, 113690, 113691, 113692, 113693, 113694, 113695, 113696, 113697, 113698, 113699 were selected from the ORFs having the nucleotide sequence represented by SEQ ID NO: 78106.

The probes having the nucleotide sequence represented by SEQ ID NOS: 113700, 113701, 113702, 113703, 113704, 113705, 113706, 113707, 113708, 113709, 113710 were selected from the ORFs having the nucleotide sequence represented by SEQ ID NO: 78107.

The probes having the nucleotide sequence represented by SEQ ID NOS: 113711, 113712, 113713, 113714, 113715, 113716, 113717, 113718, 113719, 113720, 113721 were selected from the ORFs having the nucleotide sequence represented by SEQ ID NO: 78108.

The probes having the nucleotide sequence represented by SEQ ID NOS: 113722, 113723, 113724, 113725, 113726, 113727, 113728, 113729, 113730, 113731, 113732 were selected from the ORFs having the nucleotide sequence represented by SEQ ID NO: 78109.

The probes having the nucleotide sequence represented by SEQ ID NOS: 113733, 113734, 113735, 113736, 113737, 113738, 113739, 113740, 113741, 113742, 113743 were selected from the ORFs having the nucleotide sequence represented by SEQ ID NO: 78110.

The probes having the nucleotide sequence represented by SEQ ID NOS: 113744, 113745, 113746, 113747, 113748, 113749, 113750, 113751, 113752, 113753, 113754 were selected from the ORFs having the nucleotide sequence represented by SEQ ID NO: 78111.

The probes having the nucleotide sequence represented by SEQ ID NOS: 113755, 113756, 113757, 113758, 113759, 113760, 113761, 113762, 113763, 113764, 113765 were selected from the ORFs having the nucleotide sequence represented by SEQ ID NO: 78112.

The probes having the nucleotide sequence represented by SEQ ID NOS: 113766, 113767, 113768, 113769, 113770, 113771, 113772, 113773, 113774, 113775, 113776 were selected from the ORFs having the nucleotide sequence represented by SEQ ID NO: 78113.

The probes having the nucleotide sequence represented by SEQ ID NOS: 113777, 113778, 113779, 113780, 113781, 113782, 113783, 113784, 113785, 113786, 113787 were selected from the ORFs having the nucleotide sequence represented by SEQ ID NO: 78114.

The probes having the nucleotide sequence represented by SEQ ID NOS: 113788, 113789, 113790, 113791, 113792, 113793, 113794, 113795, 113796, 113797, 113798 were selected from the ORFs having the nucleotide sequence represented by SEQ ID NO: 78115.

The probes having the nucleotide sequence represented by SEQ ID NOS: 113799, 113800, 113801, 113802, 113803, 113804, 113805, 113806, 113807, 113808, 113809 were selected from the ORFs having the nucleotide sequence represented by SEQ ID NO: 78116.

The probes having the nucleotide sequence represented by SEQ ID NOS: 113810, 113811, 113812, 113813, 113814, 113815, 113816, 113817, 113818, 113819, 113820 were selected from the ORFs having the nucleotide sequence represented by SEQ ID NO: 78117.

The probes having the nucleotide sequence represented by SEQ ID NOS: 113821, 113822, 113823, 113824, 113825, 113826, 113827, 113828, 113829, 113830, 113831 were selected from the ORFs having the nucleotide sequence represented by SEQ ID NO: 78118.

The probes having the nucleotide sequence represented by SEQ ID NOS: 113832, 113833, 113834, 113835, 113836, 113837, 113838, 113839, 113840, 113841, 113842 were selected from the ORFs having the nucleotide sequence represented by SEQ ID NO: 78119.

The probes having the nucleotide sequence represented by SEQ ID NOS: 113843, 113844, 113845, 113846, 113847, 113848, 113849, 113850, 113851, 113852, 113853 were selected from the ORFs having the nucleotide sequence represented by SEQ ID NO: 78120.

The probes having the nucleotide sequence represented by SEQ ID NOS: 113854, 113855, 113856, 113857, 113858, 113859, 113860, 113861, 113862, 113863, 113864 were selected from the ORFs having the nucleotide sequence represented by SEQ ID NO: 78121.

The probes having the nucleotide sequence represented by SEQ ID NOS: 113865, 113866, 113867, 113868, 113869, 113870, 113871, 113872, 113873, 113874, 113875 were selected from the ORFs having the nucleotide sequence represented by SEQ ID NO: 78122.

The probes having the nucleotide sequence represented by SEQ ID NOS: 113876, 113877, 113878, 113879, 113880, 113881, 113882, 113883, 113884, 113885, 113886 were selected from the ORFs having the nucleotide sequence represented by SEQ ID NO: 78123.

The probes having the nucleotide sequence represented by SEQ ID NOS: 113887, 113888, 113889, 113890, 113891, 113892, 113893, 113894, 113895, 113896, 113897 were selected from the ORFs having the nucleotide sequence represented by SEQ ID NO: 78124.

The probes having the nucleotide sequence represented by SEQ ID NOS: 113898, 113899, 113900, 113901, 113902, 113903, 113904, 113905, 113906, 113907, 113908 were selected from the ORFs having the nucleotide sequence represented by SEQ ID NO: 78125.

The probes having the nucleotide sequence represented by SEQ ID NOS: 113909, 113910, 113911, 113912, 113913, 113914, 113915, 113916, 113917, 113918, 113919 were selected from the ORFs having the nucleotide sequence represented by SEQ ID NO: 78126.

The probes having the nucleotide sequence represented by SEQ ID NOS: 113920, 113921, 113922, 113923, 113924, 113925, 113926, 113927, 113928, 113929, 113930 were selected from the ORFs having the nucleotide sequence represented by SEQ ID NO: 78127.

The probes having the nucleotide sequence represented by SEQ ID NOS: 113931, 113932, 113933, 113934, 113935, 113936, 113937, 113938, 113939, 113940, 113941 were selected from the ORFs having the nucleotide sequence represented by SEQ ID NO: 78128.

The probes having the nucleotide sequence represented by SEQ ID NOS: 113942, 113943, 113944, 113945, 113946, 113947, 113948, 113949, 113950, 113951, 113952 were selected from the ORFs having the nucleotide sequence represented by SEQ ID NO: 78129.

The probes having the nucleotide sequence represented by SEQ ID NOS: 113953, 113954, 113955, 113956, 113957, 113958, 113959, 113960, 113961, 113962, 113963 were selected from the ORFs having the nucleotide sequence represented by SEQ ID NO: 78130.

The probes having the nucleotide sequence represented by SEQ ID NOS: 113964, 113965, 113966, 113967, 113968, 113969, 113970, 113971, 113972, 113973, 113974 were selected from the ORFs having the nucleotide sequence represented by SEQ ID NO: 78131.

The probes having the nucleotide sequence represented by SEQ ID NOS: 113975, 113976, 113977, 113978, 113979, 113980, 113981, 113982, 113983, 113984, 113985 were selected from the ORFs having the nucleotide sequence represented by SEQ ID NO: 78132.

The probes having the nucleotide sequence represented by SEQ ID NOS: 113986, 113987, 113988, 113989, 113990, 113991, 113992, 113993, 113994, 113995, 113996 were selected from the ORFs having the nucleotide sequence represented by SEQ ID NO: 78133.

The probes having the nucleotide sequence represented by SEQ ID NOS: 113997, 113998, 113999, 114000, 114001, 114002, 114003, 114004, 114005, 114006, 114007 were selected from the ORFs having the nucleotide sequence represented by SEQ ID NO: 78134.

The probes having the nucleotide sequence represented by SEQ ID NOS: 114008, 114009, 114010, 114011, 114012, 114013, 114014, 114015, 114016, 114017, 114018 were selected from the ORFs having the nucleotide sequence represented by SEQ ID NO: 78135.

The probes having the nucleotide sequence represented by SEQ ID NOS: 114019, 114020, 114021, 114022, 114023, 114024, 114025, 114026, 114027, 114028, 114029 were selected from the ORFs having the nucleotide sequence represented by SEQ ID NO: 78136.

The probes having the nucleotide sequence represented by SEQ ID NOS: 114030, 114031, 114032, 114033, 114034, 114035, 114036, 114037, 114038, 114039, 114040 were selected from the ORFs having the nucleotide sequence represented by SEQ ID NO: 78137.

The probes having the nucleotide sequence represented by SEQ ID NOS: 114041, 114042, 114043, 114044, 114045, 114046, 114047, 114048, 114049, 114050, 114051 were selected from the ORFs having the nucleotide sequence represented by SEQ ID NO: 78138.

The probes having the nucleotide sequence represented by SEQ ID NOS: 114052, 114053, 114054, 114055, 114056, 114057, 114058, 114059, 114060, 114061, 114062 were selected from the ORFs having the nucleotide sequence represented by SEQ ID NO: 78139.

The probes having the nucleotide sequence represented by SEQ ID NOS: 114063, 114064, 114065, 114066, 114067, 114068, 114069, 114070, 114071, 114072, 114073 were selected from the ORFs having the nucleotide sequence represented by SEQ ID NO: 78140.

The probes having the nucleotide sequence represented by SEQ ID NOS: 114074, 114075, 114076, 114077, 114078, 114079, 114080, 114081, 114082, 114083, 114084 were selected from the ORFs having the nucleotide sequence represented by SEQ ID NO: 78141.

The probes having the nucleotide sequence represented by SEQ ID NOS: 114085, 114086, 114087, 114088, 114089, 114090, 114091, 114092, 114093, 114094, 114095 were selected from the ORFs having the nucleotide sequence represented by SEQ ID NO: 78142.

The probes having the nucleotide sequence represented by SEQ ID NOS: 114096, 114097, 114098, 114099, 114100, 114101, 114102, 114103, 114104, 114105, 114106 were selected from the ORFs having the nucleotide sequence represented by SEQ ID NO: 78143.

The probes having the nucleotide sequence represented by SEQ ID NOS: 114107, 114108, 114109, 114110, 114111, 114112, 114113, 114114, 114115, 114116, 114117 were selected from the ORFs having the nucleotide sequence represented by SEQ ID NO: 78144.

The probes having the nucleotide sequence represented by SEQ ID NOS: 114118, 114119, 114120, 114121, 114122, 114123, 114124, 114125, 114126, 114127, 114128 were selected from the ORFs having the nucleotide sequence represented by SEQ ID NO: 78145.

The probes having the nucleotide sequence represented by SEQ ID NOS: 114129, 114130, 114131, 114132, 114133, 114134, 114135, 114136, 114137, 114138, 114139 were selected from the ORFs having the nucleotide sequence represented by SEQ ID NO: 78146.

The probes having the nucleotide sequence represented by SEQ ID NOS: 114140, 114141, 114142, 114143, 114144, 114145, 114146, 114147, 114148, 114149, 114150 were selected from the ORFs having the nucleotide sequence represented by SEQ ID NO: 78147.

The probes having the nucleotide sequence represented by SEQ ID NOS: 114151, 114152, 114153, 114154, 114155, 114156, 114157, 114158, 114159, 114160, 114161 were selected from the ORFs having the nucleotide sequence represented by SEQ ID NO: 78148.

The probes having the nucleotide sequence represented by SEQ ID NOS: 114162, 114163, 114164, 114165, 114166, 114167, 114168, 114169, 114170, 114171, 114172 were selected from the ORFs having the nucleotide sequence represented by SEQ ID NO: 78149.

The probes having the nucleotide sequence represented by SEQ ID NOS: 114173, 114174, 114175, 114176, 114177, 114178, 114179, 114180, 114181, 114182, 114183 were selected from the ORFs having the nucleotide sequence represented by SEQ ID NO: 78150.

The probes having the nucleotide sequence represented by SEQ ID NOS: 114184, 114185, 114186, 114187, 114188, 114189, 114190, 114191, 114192, 114193, 114194 were selected from the ORFs having the nucleotide sequence represented by SEQ ID NO: 78151.

The probes having the nucleotide sequence represented by SEQ ID NOS: 114195, 114196, 114197, 114198, 114199, 114200, 114201, 114202, 114203, 114204, 114205 were selected from the ORFs having the nucleotide sequence represented by SEQ ID NO: 78152.

The probes having the nucleotide sequence represented by SEQ ID NOS: 114206, 114207, 114208, 114209, 114210, 114211, 114212, 114213, 114214, 114215, 114216 were selected from the ORFs having the nucleotide sequence represented by SEQ ID NO: 78153.

The probes having the nucleotide sequence represented by SEQ ID NOS: 114217, 114218, 114219, 114220, 114221, 114222, 114223, 114224, 114225, 114226, 114227 were selected from the ORFs having the nucleotide sequence represented by SEQ ID NO: 78154.

The probes having the nucleotide sequence represented by SEQ ID NOS: 114228, 114229, 114230, 114231, 114232, 114233, 114234, 114235, 114236, 114237, 114238 were selected from the ORFs having the nucleotide sequence represented by SEQ ID NO: 78155.

The probes having the nucleotide sequence represented by SEQ ID NOS: 114239, 114240, 114241, 114242, 114243, 114244, 114245, 114246, 114247, 114248, 114249 were selected from the ORFs having the nucleotide sequence represented by SEQ ID NO: 78156.

The probes having the nucleotide sequence represented by SEQ ID NOS: 114250, 114251, 114252, 114253, 114254, 114255, 114256, 114257, 114258, 114259, 114260 were selected from the ORFs having the nucleotide sequence represented by SEQ ID NO: 78157.

The probes having the nucleotide sequence represented by SEQ ID NOS: 114261, 114262, 114263, 114264, 114265, 114266, 114267, 114268, 114269, 114270, 114271 were selected from the ORFs having the nucleotide sequence represented by SEQ ID NO: 78158.

The probes having the nucleotide sequence represented by SEQ ID NOS: 114272, 114273, 114274, 114275, 114276, 114277, 114278, 114279, 114280, 114281, 114282 were selected from the ORFs having the nucleotide sequence represented by SEQ ID NO: 78159.

The probes having the nucleotide sequence represented by SEQ ID NOS: 114283, 114284, 114285, 114286, 114287, 114288, 114289, 114290, 114291, 114292, 114293 were selected from the ORFs having the nucleotide sequence represented by SEQ ID NO: 78160.

The probes having the nucleotide sequence represented by SEQ ID NOS: 114294, 114295, 114296, 114297, 114298, 114299, 114300, 114301, 114302, 114303, 114304 were selected from the ORFs having the nucleotide sequence represented by SEQ ID NO: 78161.

The probes having the nucleotide sequence represented by SEQ ID NOS: 114305, 114306, 114307, 114308, 114309, 114310, 114311, 114312, 114313, 114314, 114315 were selected from the ORFs having the nucleotide sequence represented by SEQ ID NO: 78162.

The probes having the nucleotide sequence represented by SEQ ID NOS: 114316, 114317, 114318, 114319, 114320, 114321, 114322, 114323, 114324, 114325, 114326 were selected from the ORFs having the nucleotide sequence represented by SEQ ID NO: 78163.

The probes having the nucleotide sequence represented by SEQ ID NOS: 114327, 114328, 114329, 114330, 114331, 114332, 114333, 114334, 114335, 114336, 114337 were selected from the ORFs having the nucleotide sequence represented by SEQ ID NO: 78164.

The probes having the nucleotide sequence represented by SEQ ID NOS: 114338, 114339, 114340, 114341, 114342, 114343, 114344, 114345, 114346, 114347, 114348 were selected from the ORFs having the nucleotide sequence represented by SEQ ID NO: 78165.

The probes having the nucleotide sequence represented by SEQ ID NOS: 114349, 114350, 114351, 114352, 114353, 114354, 114355, 114356, 114357, 114358, 114359 were selected from the ORFs having the nucleotide sequence represented by SEQ ID NO: 78166.

The probes having the nucleotide sequence represented by SEQ ID NOS: 114360, 114361, 114362, 114363, 114364, 114365, 114366, 114367, 114368, 114369, 114370 were selected from the ORFs having the nucleotide sequence represented by SEQ ID NO: 78167.

The probes having the nucleotide sequence represented by SEQ ID NOS: 114371, 114372, 114373, 114374, 114375, 114376, 114377, 114378, 114379, 114380, 114381 were selected from the ORFs having the nucleotide sequence represented by SEQ ID NO: 78168.

The probes having the nucleotide sequence represented by SEQ ID NOS: 114382, 114383, 114384, 114385, 114386, 114387, 114388, 114389, 114390, 114391, 114392 were selected from the ORFs having the nucleotide sequence represented by SEQ ID NO: 78169.

The probes having the nucleotide sequence represented by SEQ ID NOS: 114393, 114394, 114395, 114396, 114397, 114398, 114399, 114400, 114401, 114402, 114403 were selected from the ORFs having the nucleotide sequence represented by SEQ ID NO: 78170.

The probes having the nucleotide sequence represented by SEQ ID NOS: 114404, 114405, 114406, 114407, 114408, 114409, 114410, 114411, 114412, 114413, 114414 were selected from the ORFs having the nucleotide sequence represented by SEQ ID NO: 78171.

The probes having the nucleotide sequence represented by SEQ ID NOS: 114415, 114416, 114417, 114418, 114419, 114420, 114421, 114422, 114423, 114424, 114425 were selected from the ORFs having the nucleotide sequence represented by SEQ ID NO: 78172.

The probes having the nucleotide sequence represented by SEQ ID NOS: 114426, 114427, 114428, 114429, 114430, 114431, 114432, 114433, 114434, 114435, 114436 were selected from the ORFs having the nucleotide sequence represented by SEQ ID NO: 78173.

The probes having the nucleotide sequence represented by SEQ ID NOS: 114437, 114438, 114439, 114440, 114441, 114442, 114443, 114444, 114445, 114446, 114447 were selected from the ORFs having the nucleotide sequence represented by SEQ ID NO: 78174.

The probes having the nucleotide sequence represented by SEQ ID NOS: 114448, 114449, 114450, 114451, 114452, 114453, 114454, 114455, 114456, 114457, 114458 were selected from the ORFs having the nucleotide sequence represented by SEQ ID NO: 78175.

The probes having the nucleotide sequence represented by SEQ ID NOS: 114459, 114460, 114461, 114462, 114463, 114464, 114465, 114466, 114467, 114468, 114469 were selected from the ORFs having the nucleotide sequence represented by SEQ ID NO: 78176.

The probes having the nucleotide sequence represented by SEQ ID NOS: 114470, 114471, 114472, 114473, 114474, 114475, 114476, 114477, 114478, 114479, 114480 were selected from the ORFs having the nucleotide sequence represented by SEQ ID NO: 78177.

The probes having the nucleotide sequence represented by SEQ ID NOS: 114481, 114482, 114483, 114484, 114485, 114486, 114487, 114488, 114489, 114490, 114491 were selected from the ORFs having the nucleotide sequence represented by SEQ ID NO: 78178.

The probes having the nucleotide sequence represented by SEQ ID NOS: 114492, 114493, 114494, 114495, 114496, 114497, 114498, 114499, 114500, 114501, 114502 were selected from the ORFs having the nucleotide sequence represented by SEQ ID NO: 78179.

The probes having the nucleotide sequence represented by SEQ ID NOS: 114503, 114504, 114505, 114506, 114507, 114508, 114509, 114510, 114511, 114512, 114513 were selected from the ORFs having the nucleotide sequence represented by SEQ ID NO: 78180.

The probes having the nucleotide sequence represented by SEQ ID NOS: 114514, 114515, 114516, 114517, 114518, 114519, 114520, 114521, 114522, 114523, 114524 were selected from the ORFs having the nucleotide sequence represented by SEQ ID NO: 78181.

The probes having the nucleotide sequence represented by SEQ ID NOS: 114525, 114526, 114527, 114528, 114529, 114530, 114531, 114532, 114533, 114534, 114535 were selected from the ORFs having the nucleotide sequence represented by SEQ ID NO: 78182.

The probes having the nucleotide sequence represented by SEQ ID NOS: 114536, 114537, 114538, 114539, 114540, 114541, 114542, 114543, 114544, 114545, 114546 were selected from the ORFs having the nucleotide sequence represented by SEQ ID NO: 78183.

The probes having the nucleotide sequence represented by SEQ ID NOS: 114547, 114548, 114549, 114550, 114551, 114552, 114553, 114554, 114555, 114556, 114557 were selected from the ORFs having the nucleotide sequence represented by SEQ ID NO: 78184.

The probes having the nucleotide sequence represented by SEQ ID NOS: 114558, 114559, 114560, 114561, 114562, 114563, 114564, 114565, 114566, 114567, 114568 were selected from the ORFs having the nucleotide sequence represented by SEQ ID NO: 78185.

The probes having the nucleotide sequence represented by SEQ ID NOS: 114569, 114570, 114571, 114572, 114573, 114574, 114575, 114576, 114577, 114578, 114579 were selected from the ORFs having the nucleotide sequence represented by SEQ ID NO: 78186.

The probes having the nucleotide sequence represented by SEQ ID NOS: 114580, 114581, 114582, 114583, 114584, 114585, 114586, 114587, 114588, 114589, 114590 were selected from the ORFs having the nucleotide sequence represented by SEQ ID NO: 78187.

The probes having the nucleotide sequence represented by SEQ ID NOS: 114591, 114592, 114593, 114594, 114595, 114596, 114597, 114598, 114599, 114600, 114601 were selected from the ORFs having the nucleotide sequence represented by SEQ ID NO: 78188.

The probes having the nucleotide sequence represented by SEQ ID NOS: 114602, 114603, 114604, 114605, 114606, 114607, 114608, 114609, 114610, 114611, 114612 were selected from the ORFs having the nucleotide sequence represented by SEQ ID NO: 78189.

The probes having the nucleotide sequence represented by SEQ ID NOS: 114613, 114614, 114615, 114616, 114617, 114618, 114619, 114620, 114621, 114622, 114623 were selected from the ORFs having the nucleotide sequence represented by SEQ ID NO: 78190.

The probes having the nucleotide sequence represented by SEQ ID NOS: 114624, 114625, 114626, 114627, 114628, 114629, 114630, 114631, 114632, 114633, 114634 were selected from the ORFs having the nucleotide sequence represented by SEQ ID NO: 78191.

The probes having the nucleotide sequence represented by SEQ ID NOS: 114635, 114636, 114637, 114638, 114639, 114640, 114641, 114642, 114643, 114644, 114645 were selected from the ORFs having the nucleotide sequence represented by SEQ ID NO: 78192.

The probes having the nucleotide sequence represented by SEQ ID NOS: 114646, 114647, 114648, 114649, 114650, 114651, 114652, 114653, 114654, 114655, 114656 were selected from the ORFs having the nucleotide sequence represented by SEQ ID NO: 78193.

The probes having the nucleotide sequence represented by SEQ ID NOS: 114657, 114658, 114659, 114660, 114661, 114662, 114663, 114664, 114665, 114666, 114667 were selected from the ORFs having the nucleotide sequence represented by SEQ ID NO: 78194.

The probes having the nucleotide sequence represented by SEQ ID NOS: 114668, 114669, 114670, 114671, 114672, 114673, 114674, 114675, 114676, 114677, 114678 were selected from the ORFs having the nucleotide sequence represented by SEQ ID NO: 78195.

The probes having the nucleotide sequence represented by SEQ ID NOS: 114679, 114680, 114681, 114682, 114683, 114684, 114685, 114686, 114687, 114688, 114689 were selected from the ORFs having the nucleotide sequence represented by SEQ ID NO: 78196.

The probes having the nucleotide sequence represented by SEQ ID NOS: 114690, 114691, 114692, 114693, 114694, 114695, 114696, 114697, 114698, 114699, 114700 were selected from the ORFs having the nucleotide sequence represented by SEQ ID NO: 78197.

The probes having the nucleotide sequence represented by SEQ ID NOS: 114701, 114702, 114703, 114704, 114705, 114706, 114707, 114708, 114709, 114710, 114711 were selected from the ORFs having the nucleotide sequence represented by SEQ ID NO: 78198.

The probes having the nucleotide sequence represented by SEQ ID NOS: 114712, 114713, 114714, 114715, 114716, 114717, 114718, 114719, 114720, 114721, 114722 were selected from the ORFs having the nucleotide sequence represented by SEQ ID NO: 78199.

The probes having the nucleotide sequence represented by SEQ ID NOS: 114723, 114724, 114725, 114726, 114727, 114728, 114729, 114730, 114731, 114732, 114733 were selected from the ORFs having the nucleotide sequence represented by SEQ ID NO: 78200.

The probes having the nucleotide sequence represented by SEQ ID NOS: 114734, 114735, 114736, 114737, 114738, 114739, 114740, 114741, 114742, 114743, 114744 were selected from the ORFs having the nucleotide sequence represented by SEQ ID NO: 78201.

The probes having the nucleotide sequence represented by SEQ ID NOS: 114745, 114746, 114747, 114748, 114749, 114750, 114751, 114752, 114753, 114754, 114755 were selected from the ORFs having the nucleotide sequence represented by SEQ ID NO: 78202.

The probes having the nucleotide sequence represented by SEQ ID NOS: 114756, 114757, 114758, 114759, 114760, 114761, 114762, 114763, 114764, 114765, 114766 were selected from the ORFs having the nucleotide sequence represented by SEQ ID NO: 78203.

The probes having the nucleotide sequence represented by SEQ ID NOS: 114767, 114768, 114769, 114770, 114771, 114772, 114773, 114774, 114775, 114776, 114777 were selected from the ORFs having the nucleotide sequence represented by SEQ ID NO: 78204.

The probes having the nucleotide sequence represented by SEQ ID NOS: 114778, 114779, 114780, 114781, 114782, 114783, 114784, 114785, 114786, 114787, 114788 were selected from the ORFs having the nucleotide sequence represented by SEQ ID NO: 78205.

The probes having the nucleotide sequence represented by SEQ ID NOS: 114789, 114790, 114791, 114792, 114793, 114794, 114795, 114796, 114797, 114798, 114799 were selected from the ORFs having the nucleotide sequence represented by SEQ ID NO: 78206.

The probes having the nucleotide sequence represented by SEQ ID NOS: 114800, 114801, 114802, 114803, 114804, 114805, 114806, 114807, 114808, 114809, 114810 were selected from the ORFs having the nucleotide sequence represented by SEQ ID NO: 78207.

The probes having the nucleotide sequence represented by SEQ ID NOS: 114811, 114812, 114813, 114814, 114815, 114816, 114817, 114818, 114819, 114820, 114821 were selected from the ORFs having the nucleotide sequence represented by SEQ ID NO: 78208.

The probes having the nucleotide sequence represented by SEQ ID NOS: 114822, 114823, 114824, 114825, 114826, 114827, 114828, 114829, 114830, 114831, 114832 were selected from the ORFs having the nucleotide sequence represented by SEQ ID NO: 78209.

The probes having the nucleotide sequence represented by SEQ ID NOS: 114833, 114834, 114835, 114836, 114837, 114838, 114839, 114840, 114841, 114842, 114843 were selected from the ORFs having the nucleotide sequence represented by SEQ ID NO: 78210.

The probes having the nucleotide sequence represented by SEQ ID NOS: 114844, 114845, 114846, 114847, 114848, 114849, 114850, 114851, 114852, 114853, 114854 were selected from the ORFs having the nucleotide sequence represented by SEQ ID NO: 78211.

The probes having the nucleotide sequence represented by SEQ ID NOS: 114855, 114856, 114857, 114858, 114859, 114860, 114861, 114862, 114863, 114864, 114865 were selected from the ORFs having the nucleotide sequence represented by SEQ ID NO: 78212.

The probes having the nucleotide sequence represented by SEQ ID NOS: 114866, 114867, 114868, 114869, 114870, 114871, 114872, 114873, 114874, 114875, 114876 were selected from the ORFs having the nucleotide sequence represented by SEQ ID NO: 78213.

The probes having the nucleotide sequence represented by SEQ ID NOS: 114877, 114878, 114879, 114880, 114881, 114882, 114883, 114884, 114885, 114886, 114887 were selected from the ORFs having the nucleotide sequence represented by SEQ ID NO: 78214.

The probes having the nucleotide sequence represented by SEQ ID NOS: 114888, 114889, 114890, 114891, 114892, 114893, 114894, 114895, 114896, 114897, 114898 were selected from the ORFs having the nucleotide sequence represented by SEQ ID NO: 78215.

The probes having the nucleotide sequence represented by SEQ ID NOS: 114899, 114900, 114901, 114902, 114903, 114904, 114905, 114906, 114907, 114908, 114909 were selected from the ORFs having the nucleotide sequence represented by SEQ ID NO: 78216.

The probes having the nucleotide sequence represented by SEQ ID NOS: 114910, 114911, 114912, 114913, 114914, 114915, 114916, 114917, 114918, 114919, 114920 were selected from the ORFs having the nucleotide sequence represented by SEQ ID NO: 78217.

The probes having the nucleotide sequence represented by SEQ ID NOS: 114921, 114922, 114923, 114924, 114925, 114926, 114927, 114928, 114929, 114930, 114931 were selected from the ORFs having the nucleotide sequence represented by SEQ ID NO: 78218.

The probes having the nucleotide sequence represented by SEQ ID NOS: 114932, 114933, 114934, 114935, 114936, 114937, 114938, 114939, 114940, 114941, 114942 were selected from the ORFs having the nucleotide sequence represented by SEQ ID NO: 78219.

The probes having the nucleotide sequence represented by SEQ ID NOS: 114943, 114944, 114945, 114946, 114947, 114948, 114949, 114950, 114951, 114952, 114953 were selected from the ORFs having the nucleotide sequence represented by SEQ ID NO: 78220.

The probes having the nucleotide sequence represented by SEQ ID NOS: 114954, 114955, 114956, 114957, 114958, 114959, 114960, 114961, 114962, 114963, 114964 were selected from the ORFs having the nucleotide sequence represented by SEQ ID NO: 78221.

The probes having the nucleotide sequence represented by SEQ ID NOS: 114965, 114966, 114967, 114968, 114969, 114970, 114971, 114972, 114973, 114974, 114975 were selected from the ORFs having the nucleotide sequence represented by SEQ ID NO: 78222.

The probes having the nucleotide sequence represented by SEQ ID NOS: 114976, 114977, 114978, 114979, 114980, 114981, 114982, 114983, 114984, 114985, 114986 were selected from the ORFs having the nucleotide sequence represented by SEQ ID NO: 78223.

The probes having the nucleotide sequence represented by SEQ ID NOS: 114987, 114988, 114989, 114990, 114991, 114992, 114993, 114994, 114995, 114996, 114997 were selected from the ORFs having the nucleotide sequence represented by SEQ ID NO: 78224.

The probes having the nucleotide sequence represented by SEQ ID NOS: 114998, 114999, 115000, 115001, 115002, 115003, 115004, 115005, 115006, 115007, 115008 were selected from the ORFs having the nucleotide sequence represented by SEQ ID NO: 78225.

The probes having the nucleotide sequence represented by SEQ ID NOS: 115009, 115010, 115011, 115012, 115013, 115014, 115015, 115016, 115017, 115018, 115019 were selected from the ORFs having the nucleotide sequence represented by SEQ ID NO: 78226.

The probes having the nucleotide sequence represented by SEQ ID NOS: 115020, 115021, 115022, 115023, 115024, 115025, 115026, 115027, 115028, 115029, 115030 were selected from the ORFs having the nucleotide sequence represented by SEQ ID NO: 78227.

The probes having the nucleotide sequence represented by SEQ ID NOS: 115031, 115032, 115033, 115034, 115035, 115036, 115037, 115038, 115039, 115040, 115041 were selected from the ORFs having the nucleotide sequence represented by SEQ ID NO: 78228.

The probes having the nucleotide sequence represented by SEQ ID NOS: 115042, 115043, 115044, 115045, 115046, 115047, 115048, 115049, 115050, 115051, 115052 were selected from the ORFs having the nucleotide sequence represented by SEQ ID NO: 78229.

The probes having the nucleotide sequence represented by SEQ ID NOS: 115053, 115054, 115055, 115056, 115057, 115058, 115059, 115060, 115061, 115062, 115063 were selected from the ORFs having the nucleotide sequence represented by SEQ ID NO: 78230.

The probes having the nucleotide sequence represented by SEQ ID NOS:.115064, 115065, 115066, 115067, 115068, 115069, 115070, 115071, 115072, 115073, 115074 were selected from the ORFs having the nucleotide sequence represented by SEQ ID NO: 78231.

The probes having the nucleotide sequence represented by SEQ ID NOS: 115075, 115076, 115077, 115078, 115079, 115080, 115081, 115082, 115083, 115084, 115085 were selected from the ORFs having the nucleotide sequence represented by SEQ ID NO: 78232.

The probes having the nucleotide sequence represented by SEQ ID NOS: 115086, 115087, 115088, 115089, 115090, 115091, 115092, 115093, 115094, 115095, 115096 were selected from the ORFs having the nucleotide sequence represented by SEQ ID NO: 78233.

The probes having the nucleotide sequence represented by SEQ ID NOS: 115097, 115098, 115099, 115100, 115101, 115102, 115103, 115104, 115105, 115106, 115107 were selected from the ORFs having the nucleotide sequence represented by SEQ ID NO: 78234.

The probes having the nucleotide sequence represented by SEQ ID NOS: 115108, 115109, 115110, 115111, 115112, 115113, 115114, 115115, 115116, 115117, 115118 were selected from the ORFs having the nucleotide sequence represented by SEQ ID NO: 78235.

The probes having the nucleotide sequence represented by SEQ ID NOS: 115119, 115120, 115121, 115122, 115123, 115124, 115125, 115126, 115127, 115128, 115129 were selected from the ORFs having the nucleotide sequence represented by SEQ ID NO: 78236.

The probes having the nucleotide sequence represented by SEQ ID NOS: 115130, 115131, 115132, 115133, 115134, 115135, 115136, 115137, 115138, 115139, 115140 were selected from the ORFs having the nucleotide sequence represented by SEQ ID NO: 78237.

The probes having the nucleotide sequence represented by SEQ ID NOS: 115141, 115142, 115143, 115144, 115145, 115146, 115147, 115148, 115149, 115150, 115151 were selected from the ORFs having the nucleotide sequence represented by SEQ ID NO: 78238.

The probes having the nucleotide sequence represented by SEQ ID NOS: 115152, 115153, 115154, 115155, 115156, 115157, 115158, 115159, 115160, 115161, 115162 were selected from the ORFs having the nucleotide sequence represented by SEQ ID NO: 78239.

The probes having the nucleotide sequence represented by SEQ ID NOS: 115163, 115164, 115165, 115166, 115167, 115168, 115169, 115170, 115171, 115172, 115173 were selected from the ORFs having the nucleotide sequence represented by SEQ ID NO: 78240.

The probes having the nucleotide sequence represented by SEQ ID NOS: 115174, 115175, 115176, 115177, 115178, 115179, 115180, 115181, 115182, 115183, 115184 were selected from the ORFs having the nucleotide sequence represented by SEQ ID NO: 78241.

The probes having the nucleotide sequence represented by SEQ ID NOS: 115185, 115186, 115187, 115188, 115189, 115190, 115191, 115192, 115193, 115194, 115195 were selected from the ORFs having the nucleotide sequence represented by SEQ ID NO: 78242.

The probes having the nucleotide sequence represented by SEQ ID NOS: 115196, 115197, 115198, 115199, 115200, 115201, 115202, 115203, 115204, 115205, 115206 were selected from the ORFs having the nucleotide sequence represented by SEQ ID NO: 78243.

The probes having the nucleotide sequence represented by SEQ ID NOS: 115207, 115208, 115209, 115210, 115211, 115212, 115213, 115214, 115215, 115216, 115217 were selected from the ORFs having the nucleotide sequence represented by SEQ ID NO: 78244.

The probes having the nucleotide sequence represented by SEQ ID NOS: 115218, 115219, 115220, 115221, 115222, 115223, 115224, 115225, 115226, 115227, 115228 were selected from the ORFs having the nucleotide sequence represented by SEQ ID NO: 78245.

The probes having the nucleotide sequence represented by SEQ ID NOS: 115229, 115230, 115231, 115232, 115233, 115234, 115235, 115236, 115237, 115238, 115239, 115240, 115241, 115242, 115243, 115244, 115245, 115246, 115247, 115248, 115249, 115250 were selected from the ORFs having the nucleotide sequence represented by SEQ ID NO: 78246.

The probes having the nucleotide sequence represented by SEQ ID NOS: 115251, 115252, 115253, 115254, 115255, 115256, 115257, 115258, 115259, 115260, 115261 were selected from the ORFs having the nucleotide sequence represented by SEQ ID NO: 78247.

The probes having the nucleotide sequence represented by SEQ ID NOS: 115262, 115263, 115264, 115265, 115266, 115267, 115268, 115269, 115270, 115271, 115272 were selected from the ORFs having the nucleotide sequence represented by SEQ ID NO: 78248.

The probes having the nucleotide sequence represented by SEQ ID NOS: 115273, 115274, 115275, 115276, 115277, 115278, 115279, 115280, 115281, 115282, 115283 were selected from the ORFs having the nucleotide sequence represented by SEQ ID NO: 78249.

The probes having the nucleotide sequence represented by SEQ ID NOS: 115284, 115285, 115286, 115287, 115288, 115289, 115290, 115291, 115292, 115293, 115294 were selected from the ORFs having the nucleotide sequence represented by SEQ ID NO: 78250.

The probes having the nucleotide sequence represented by SEQ ID NOS: 115295, 115296, 115297, 115298, 115299, 115300, 115301, 115302, 115303, 115304, 115305 were selected from the ORFs having the nucleotide sequence represented by SEQ ID NO: 78251.

The probes having the nucleotide sequence represented by SEQ ID NOS: 115306, 115307, 115308, 115309, 115310, 115311, 115312, 115313, 115314, 115315, 115316 were selected from the ORFs having the nucleotide sequence represented by SEQ ID NO: 78252.

The probes having the nucleotide sequence represented by SEQ ID NOS: 115317, 115318, 115319, 115320, 115321, 115322, 115323, 115324, 115325, 115326, 115327 were selected from the ORFs having the nucleotide sequence represented by SEQ ID NO: 78253.

The probes having the nucleotide sequence represented by SEQ ID NOS: 115328, 115329, 115330, 115331, 115332, 115333, 115334, 115335, 115336, 115337, 115338 were selected from the ORFs having the nucleotide sequence represented by SEQ ID NO: 78254.

The probes having the nucleotide sequence represented by SEQ ID NOS: 115339, 115340, 115341, 115342, 115343, 115344, 115345, 115346, 115347, 115348, 115349 were selected from the ORFs having the nucleotide sequence represented by SEQ ID NO: 78255.

The probes having the nucleotide sequence represented by SEQ ID NOS: 115350, 115351, 115352, 115353, 115354, 115355, 115356, 115357, 115358, 115359, 115360 were selected from the ORFs having the nucleotide sequence represented by SEQ ID NO: 78256.

The probes having the nucleotide sequence represented by SEQ ID NOS: 115361, 115362, 115363, 115364, 115365, 115366, 115367, 115368, 115369, 115370, 115371 were selected from the ORFs having the nucleotide sequence represented by SEQ ID NO: 78257.

The probes having the nucleotide sequence represented by SEQ ID NOS: 115372, 115373, 115374, 115375, 115376, 115377, 115378, 115379, 115380, 115381, 115382 were selected from the ORFs having the nucleotide sequence represented by SEQ ID NO: 78258.

The probes having the nucleotide sequence represented by SEQ ID NOS: 115383, 115384, 115385, 115386, 115387, 115388, 115389, 115390, 115391, 115392, 115393 were selected from the ORFs having the nucleotide sequence represented by SEQ ID NO: 78259.

The probes having the nucleotide sequence represented by SEQ ID NOS: 115394, 115395, 115396, 115397, 115398, 115399, 115400, 115401, 115402, 115403, 115404 were selected from the ORFs having the nucleotide sequence represented by SEQ ID NO: 78260.

The probes having the nucleotide sequence represented by SEQ ID NOS: 115405, 115406, 115407, 115408, 115409, 115410, 115411, 115412, 115413, 115414, 115415 were selected from the ORFs having the nucleotide sequence represented by SEQ ID NO: 78261.

The probes having the nucleotide sequence represented by SEQ ID NOS: 115416, 115417, 115418, 115419, 115420, 115421, 115422, 115423, 115424, 115425, 115426 were selected from the ORFs having the nucleotide sequence represented by SEQ ID NO: 78262.

The probes having the nucleotide sequence represented by SEQ ID NOS: 115427, 115428, 115429, 115430, 115431, 115432, 115433, 115434, 115435, 115436, 115437 were selected from the ORFs having the nucleotide sequence represented by SEQ ID NO: 78263.

The probes having the nucleotide sequence represented by SEQ ID NOS: 115438, 115439, 115440, 115441, 115442, 115443, 115444, 115445, 115446, 115447, 115448 were selected from the ORFs having the nucleotide sequence represented by SEQ ID NO: 78264.

The probes having the nucleotide sequence represented by SEQ ID NOS: 115449, 115450, 115451, 115452, 115453, 115454, 115455, 115456, 115457, 115458, 115459 were selected from the ORFs having the nucleotide sequence represented by SEQ ID NO: 78265.

The probes having the nucleotide sequence represented by SEQ ID NOS: 115460, 115461, 115462, 115463, 115464, 115465, 115466, 115467, 115468, 115469, 115470 were selected from the ORFs having the nucleotide sequence represented by SEQ ID NO: 78266.

The probes having the nucleotide sequence represented by SEQ ID NOS: 115471, 115472, 115473, 115474, 115475, 115476, 115477, 115478, 115479, 115480, 115481 were selected from the ORFs having the nucleotide sequence represented by SEQ ID NO: 78267.

The probes having the nucleotide sequence represented by SEQ ID NOS: 115482, 115483, 115484, 115485, 115486, 115487, 115488, 115489, 115490, 115491, 115492 were selected from the ORFs having the nucleotide sequence represented by SEQ ID NO: 78268.

The probes having the nucleotide sequence represented by SEQ ID NOS: 115493, 115494, 115495, 115496, 115497, 115498, 115499, 115500, 115501, 115502, 115503 were selected from the ORFs having the nucleotide sequence represented by SEQ ID NO: 78269.

The probes having the nucleotide sequence represented by SEQ ID NOS: 115504, 115505, 115506, 115507, 115508, 115509, 115510, 115511, 115512, 115513, 115514 were selected from the ORFs having the nucleotide sequence represented by SEQ ID NO: 78270.

The probes having the nucleotide sequence represented by SEQ ID NOS: 115515, 115516, 115517, 115518, 115519, 115520, 115521, 115522, 115523, 115524, 115525 were selected from the ORFs having the nucleotide sequence represented by SEQ ID NO: 78271.

The probes having the nucleotide sequence represented by SEQ ID NOS: 115526, 115527, 115528, 115529, 115530, 115531, 115532, 115533, 115534, 115535, 115536 were selected from the ORFs having the nucleotide sequence represented by SEQ ID NO: 78272.

The probes having the nucleotide sequence represented by SEQ ID NOS: 115537, 115538, 115539, 115540, 115541, 115542, 115543, 115544, 115545, 115546, 115547 were selected from the ORFs having the nucleotide sequence represented by SEQ ID NO: 78273.

The probes having the nucleotide sequence represented by SEQ ID NOS: 115548, 115549, 115550, 115551, 115552, 115553, 115554, 115555, 115556, 115557, 115558 were selected from the ORFs having the nucleotide sequence represented by SEQ ID NO: 78274.

The probes having the nucleotide sequence represented by SEQ ID NOS: 115559, 115560, 115561, 115562, 115563, 115564, 115565, 115566, 115567, 115568, 115569 were selected from the ORFs having the nucleotide sequence represented by SEQ ID NO: 78275.

The probes having the nucleotide sequence represented by SEQ ID NOS: 115570, 115571, 115572, 115573, 115574, 115575, 115576, 115577, 115578, 115579, 115580 were selected from the ORFs having the nucleotide sequence represented by SEQ ID NO: 78276.

The probes having the nucleotide sequence represented by SEQ ID NOS: 115581, 115582, 115583, 115584, 115585, 115586, 115587, 115588, 115589, 115590, 115591 were selected from the ORFs having the nucleotide sequence represented by SEQ ID NO: 78277.

The probes having the nucleotide sequence represented by SEQ ID NOS: 115592, 115593, 115594, 115595, 115596, 115597, 115598, 115599, 115600, 115601, 115602 were selected from the ORFs having the nucleotide sequence represented by SEQ ID NO: 78278.

The probes having the nucleotide sequence represented by SEQ ID NOS: 115603, 115604, 115605, 115606, 115607, 115608, 115609, 115610, 115611, 115612, 115613 were selected from the ORFs having the nucleotide sequence represented by SEQ ID NO: 78279.

The probes having the nucleotide sequence represented by SEQ ID NOS: 115614, 115615, 115616, 115617, 115618, 115619, 115620, 115621, 115622, 115623, 115624 were selected from the ORFs having the nucleotide sequence represented by SEQ ID NO: 78280.

The probes having the nucleotide sequence represented by SEQ ID NOS: 115625, 115626, 115627, 115628, 115629, 115630, 115631, 115632, 115633, 115634, 115635 were selected from the ORFs having the nucleotide sequence represented by SEQ ID NO: 78281.

The probes having the nucleotide sequence represented by SEQ ID NOS: 115636, 115637, 115638, 115639, 115640, 115641, 115642, 115643, 115644, 115645, 115646 were selected from the ORFs having the nucleotide sequence represented by SEQ ID NO: 78282.

The probes having the nucleotide sequence represented by SEQ ID NOS: 115647, 115648, 115649, 115650, 115651, 115652, 115653, 115654, 115655, 115656, 115657 were selected from the ORFs having the nucleotide sequence represented by SEQ ID NO: 78283.

The probes having the nucleotide sequence represented by SEQ ID NOS: 115658, 115659, 115660, 115661, 115662, 115663, 115664, 115665, 115666, 115667, 115668 were selected from the ORFs having the nucleotide sequence represented by SEQ ID NO: 78284.

The probes having the nucleotide sequence represented by SEQ ID NOS: 115669, 115670, 115671, 115672, 115673, 115674, 115675, 115676, 115677, 115678, 115679 were selected from the ORFs having the nucleotide sequence represented by SEQ ID NO: 78285.

The probes having the nucleotide sequence represented by SEQ ID NOS: 115680, 115681, 115682, 115683, 115684, 115685, 115686, 115687, 115688, 115689, 115690 were selected from the ORFs having the nucleotide sequence represented by SEQ ID NO: 78286.

The probes having the nucleotide sequence represented by SEQ ID NOS: 115691, 115692, 115693, 115694, 115695, 115696, 115697, 115698, 115699, 115700, 115701 were selected from the ORFs having the nucleotide sequence represented by SEQ ID NO: 78287.

The probes having the nucleotide sequence represented by SEQ ID NOS: 115702, 115703, 115704, 115705, 115706, 115707, 115708, 115709, 115710, 115711, 115712 were selected from the ORFs having the nucleotide sequence represented by SEQ ID NO: 78288.

The probes having the nucleotide sequence represented by SEQ ID NOS: 115713, 115714, 115715, 115716, 115717, 115718, 115719, 115720, 115721, 115722, 115723 were selected from the ORFs having the nucleotide sequence represented by SEQ ID NO: 78289.

The probes having the nucleotide sequence represented by SEQ ID NOS: 115724, 115725, 115726, 115727, 115728, 115729, 115730, 115731, 115732, 115733, 115734 were selected from the ORFs having the nucleotide sequence represented by SEQ ID NO: 78290.

The probes having the nucleotide sequence represented by SEQ ID NOS: 115735, 115736, 115737, 115738, 115739, 115740, 115741, 115742, 115743, 115744, 115745 were selected from the ORFs having the nucleotide sequence represented by SEQ ID NO: 78291.

The probes having the nucleotide sequence represented by SEQ ID NOS: 115746, 115747, 115748, 115749, 115750, 115751, 115752, 115753, 115754, 115755, 115756 were selected from the ORFs having the nucleotide sequence represented by SEQ ID NO: 78292.

The probes having the nucleotide sequence represented by SEQ ID NOS: 115757, 115758, 115759, 115760, 115761, 115762, 115763, 115764, 115765, 115766, 115767 were selected from the ORFs having the nucleotide sequence represented by SEQ ID NO: 78293.

The probes having the nucleotide sequence represented by SEQ ID NOS: 115768, 115769, 115770, 115771, 115772, 115773, 115774, 115775, 115776, 115777, 115778 were selected from the ORFs having the nucleotide sequence represented by SEQ ID NO: 78294.

The probes having the nucleotide sequence represented by SEQ ID NOS: 115779, 115780, 115781, 115782, 115783, 115784, 115785, 115786, 115787, 115788, 115789 were selected from the ORFs having the nucleotide sequence represented by SEQ ID NO: 78295.

The probes having the nucleotide sequence represented by SEQ ID NOS: 115790, 115791, 115792, 115793, 115794, 115795, 115796, 115797, 115798, 115799, 115800 were selected from the ORFs having the nucleotide sequence represented by SEQ ID NO: 78296.

The probes having the nucleotide sequence represented by SEQ ID NOS: 115801, 115802, 115803, 115804, 115805, 115806, 115807, 115808, 115809, 115810, 115811 were selected from the ORFs having the nucleotide sequence represented by SEQ ID NO: 78297.

The probes having the nucleotide sequence represented by SEQ ID NOS: 115812, 115813, 115814, 115815, 115816, 115817, 115818, 115819, 115820, 115821, 115822 were selected from the ORFs having the nucleotide sequence represented by SEQ ID NO: 78298.

The probes having the nucleotide sequence represented by SEQ ID NOS: 115823, 115824, 115825, 115826, 115827, 115828, 115829, 115830, 115831, 115832, 115833 were selected from the ORFs having the nucleotide sequence represented by SEQ ID NO: 78299.

The probes having the nucleotide sequence represented by SEQ ID NOS: 115834, 115835, 115836, 115837, 115838, 115839, 115840, 115841, 115842, 115843, 115844 were selected from the ORFs having the nucleotide sequence represented by SEQ ID NO: 78300.

The probes having the nucleotide sequence represented by SEQ ID NOS: 115845, 115846, 115847, 115848, 115849, 115850, 115851, 115852, 115853, 115854, 115855 were selected from the ORFs having the nucleotide sequence represented by SEQ ID NO: 78301.

The probes having the nucleotide sequence represented by SEQ ID NOS: 115856, 115857, 115858, 115859, 115860, 115861, 115862, 115863, 115864, 115865, 115866 were selected from the ORFs having the nucleotide sequence represented by SEQ ID NO: 78302.

The probes having the nucleotide sequence represented by SEQ ID NOS: 115867, 115868, 115869, 115870, 115871, 115872, 115873, 115874, 115875, 115876, 115877 were selected from the ORFs having the nucleotide sequence represented by SEQ ID NO: 78303.

The probes having the nucleotide sequence represented by SEQ ID NOS: 115878, 115879, 115880, 115881, 115882, 115883, 115884, 115885, 115886, 115887, 115888 were selected from the ORFs having the nucleotide sequence represented by SEQ ID NO: 78304.

The probes having the nucleotide sequence represented by SEQ ID NOS: 115889, 115890, 115891, 115892, 115893, 115894, 115895, 115896, 115897, 115898, 115899 were selected from the ORFs having the nucleotide sequence represented by SEQ ID NO: 78305.

The probes having the nucleotide sequence represented by SEQ ID NOS: 115900, 115901, 115902, 115903, 115904, 115905, 115906, 115907, 115908, 115909, 115910 were selected from the ORFs having the nucleotide sequence represented by SEQ ID NO: 78306.

The probes having the nucleotide sequence represented by SEQ ID NOS: 115911, 115912, 115913, 115914, 115915, 115916, 115917, 115918, 115919, 115920, 115921 were selected from the ORFs having the nucleotide sequence represented by SEQ ID NO: 78307.

The probes having the nucleotide sequence represented by SEQ ID NOS: 115922, 115923, 115924, 115925, 115926, 115927, 115928, 115929, 115930, 115931, 115932 were selected from the ORFs having the nucleotide sequence represented by SEQ ID NO: 78308.

The probes having the nucleotide sequence represented by SEQ ID NOS: 115933, 115934, 115935, 115936, 115937, 115938, 115939, 115940, 115941, 115942, 115943 were selected from the ORFs having the nucleotide sequence represented by SEQ ID NO: 78309.

The probes having the nucleotide sequence represented by SEQ ID NOS: 115944, 115945, 115946, 115947, 115948, 115949, 115950, 115951, 115952, 115953, 115954 were selected from the ORFs having the nucleotide sequence represented by SEQ ID NO: 78310.

The probes having the nucleotide sequence represented by SEQ ID NOS: 115955, 115956, 115957, 115958, 115959, 115960, 115961, 115962, 115963, 115964, 115965 were selected from the ORFs having the nucleotide sequence represented by SEQ ID NO: 78311.

The probes having the nucleotide sequence represented by SEQ ID NOS: 115966, 115967, 115968, 115969, 115970, 115971, 115972, 115973, 115974, 115975, 115976 were selected from the ORFs having the nucleotide sequence represented by SEQ ID NO: 78312.

The probes having the nucleotide sequence represented by SEQ ID NOS: 115977, 115978, 115979, 115980, 115981, 115982, 115983, 115984, 115985, 115986, 115987 were selected from the ORFs having the nucleotide sequence represented by SEQ ID NO: 78313.

The probes having the nucleotide sequence represented by SEQ ID NOS: 115988, 115989, 115990, 115991, 115992, 115993, 115994, 115995, 115996, 115997, 115998 were selected from the ORFs having the nucleotide sequence represented by SEQ ID NO: 78314.

The probes having the nucleotide sequence represented by SEQ ID NOS: 115999, 116000, 116001, 116002, 116003, 116004, 116005, 116006, 116007, 116008, 116009 were selected from the ORFs having the nucleotide sequence represented by SEQ ID NO: 78315.

The probes having the nucleotide sequence represented by SEQ ID NOS: 116010, 116011, 116012, 116013, 116014, 116015, 116016, 116017, 116018, 116019, 116020 were selected from the ORFs having the nucleotide sequence represented by SEQ ID NO: 78316.

The probes having the nucleotide sequence represented by SEQ ID NOS: 116021, 116022, 116023, 116024, 116025, 116026, 116027, 116028, 116029, 116030, 116031 were selected from the ORFs having the nucleotide sequence represented by SEQ ID NO: 78317.

The probes having the nucleotide sequence represented by SEQ ID NOS: 116032, 116033, 116034, 116035, 116036, 116037, 116038, 116039, 116040, 116041, 116042 were selected from the ORFs having the nucleotide sequence represented by SEQ ID NO: 78318.

The probes having the nucleotide sequence represented by SEQ ID NOS: 116043, 116044, 116045, 116046, 116047, 116048, 116049, 116050, 116051, 116052, 116053 were selected from the ORFs having the nucleotide sequence represented by SEQ ID NO: 78319.

The probes having the nucleotide sequence represented by SEQ ID NOS: 116054, 116055, 116056, 116057, 116058, 116059, 116060, 116061, 116062, 116063, 116064 were selected from the ORFs having the nucleotide sequence represented by SEQ ID NO: 78320.

The probes having the nucleotide sequence represented by SEQ ID NOS: 116065, 116066, 116067, 116068, 116069, 116070, 116071, 116072, 116073, 116074, 116075 were selected from the ORFs having the nucleotide sequence represented by SEQ ID NO: 78321.

The probes having the nucleotide sequence represented by SEQ ID NOS: 116076, 116077, 116078, 116079, 116080, 116081, 116082, 116083, 116084, 116085, 116086 were selected from the ORFs having the nucleotide sequence represented by SEQ ID NO: 78322.

The probes having the nucleotide sequence represented by SEQ ID NOS: 116087, 116088, 116089, 116090, 116091, 116092, 116093, 116094, 116095, 116096, 116097 were selected from the ORFs having the nucleotide sequence represented by SEQ ID NO: 78323.

The probes having the nucleotide sequence represented by SEQ ID NOS: 116098, 116099, 116100, 116101, 116102, 116103, 116104, 116105, 116106, 116107, 116108 were selected from the ORFs having the nucleotide sequence represented by SEQ ID NO: 78324.

The probes having the nucleotide sequence represented by SEQ ID NOS: 116109, 116110, 116111, 116112, 116113, 116114, 116115, 116116, 116117, 116118, 116119 were selected from the ORFs having the nucleotide sequence represented by SEQ ID NO: 78325.

The probes having the nucleotide sequence represented by SEQ ID NOS: 116120, 116121, 116122, 116123, 116124, 116125, 116126, 116127, 116128, 116129, 116130 were selected from the ORFs having the nucleotide sequence represented by SEQ ID NO: 78326.

The probes having the nucleotide sequence represented by SEQ ID NOS: 116131, 116132, 116133, 116134, 116135, 116136, 116137, 116138, 116139, 116140, 116141 were selected from the ORFs having the nucleotide sequence represented by SEQ ID NO: 78327.

The probes having the nucleotide sequence represented by SEQ ID NOS: 116142, 116143, 116144, 116145, 116146, 116147, 116148, 116149, 116150, 116151, 116152 were selected from the ORFs having the nucleotide sequence represented by SEQ ID NO: 78328.

The probes having the nucleotide sequence represented by SEQ ID NOS: 116153, 116154, 116155, 116156, 116157, 116158, 116159, 116160, 116161, 116162, 116163 were selected from the ORFs having the nucleotide sequence represented by SEQ ID NO: 78329.

The probes having the nucleotide sequence represented by SEQ ID NOS: 116164, 116165, 116166, 116167, 116168, 116169, 116170, 116171, 116172, 116173, 116174 were selected from the ORFs having the nucleotide sequence represented by SEQ ID NO: 78330.

The probes having the nucleotide sequence represented by SEQ ID NOS: 116175, 116176, 116177, 116178, 116179, 116180, 116181, 116182, 116183, 116184, 116185 were selected from the ORFs having the nucleotide sequence represented by SEQ ID NO: 78331.

The probes having the nucleotide sequence represented by SEQ ID NOS: 116186, 116187, 116188, 116189, 116190, 116191, 116192, 116193, 116194, 116195, 116196 were selected from the ORFs having the nucleotide sequence represented by SEQ ID NO: 78332.

The probes having the nucleotide sequence represented by SEQ ID NOS: 116197, 116198, 116199, 116200, 116201, 116202, 116203, 116204, 116205, 116206, 116207 were selected from the ORFs having the nucleotide sequence represented by SEQ ID NO: 78333.

The probes having the nucleotide sequence represented by SEQ ID NOS: 116208, 116209, 116210, 116211, 116212, 116213, 116214, 116215, 116216, 116217, 116218 were selected from the ORFs having the nucleotide sequence represented by SEQ ID NO: 78334.

The probes having the nucleotide sequence represented by SEQ ID NOS: 116219, 116220, 116221, 116222, 116223, 116224, 116225, 116226, 116227, 116228, 116229 were selected from the ORFs having the nucleotide sequence represented by SEQ ID NO: 78335.

The probes having the nucleotide sequence represented by SEQ ID NOS: 116230, 116231, 116232, 116233, 116234, 116235, 116236, 116237, 116238, 116239, 116240 were selected from the ORFs having the nucleotide sequence represented by SEQ ID NO: 78336.

The probes having the nucleotide sequence represented by SEQ ID NOS: 116241, 116242, 116243, 116244, 116245, 116246, 116247, 116248, 116249, 116250, 116251 were selected from the ORFs having the nucleotide sequence represented by SEQ ID NO: 78337.

The probes having the nucleotide sequence represented by SEQ ID NOS: 116252, 116253, 116254, 116255, 116256, 116257, 116258, 116259, 116260, 116261, 116262 were selected from the ORFs having the nucleotide sequence represented by SEQ ID NO: 78338.

The probes having the nucleotide sequence represented by SEQ ID NOS: 116263, 116264, 116265, 116266, 116267, 116268, 116269, 116270, 116271, 116272, 116273 were selected from the ORFs having the nucleotide sequence represented by SEQ ID NO: 78339.

The probes having the nucleotide sequence represented by SEQ ID NOS: 116274, 116275, 116276, 116277, 116278, 116279, 116280, 116281, 116282, 116283, 116284 were selected from the ORFs having the nucleotide sequence represented by SEQ ID NO: 78340.

The probes having the nucleotide sequence represented by SEQ ID NOS: 116285, 116286, 116287, 116288, 116289, 116290, 116291, 116292, 116293, 116294, 116295 were selected from the ORFs having the nucleotide sequence represented by SEQ ID NO: 78341.

The probes having the nucleotide sequence represented by SEQ ID NOS: 116296, 116297, 116298, 116299, 116300, 116301, 116302, 116303, 116304, 116305, 116306 were selected from the ORFs having the nucleotide sequence represented by SEQ ID NO: 78342.

The probes having the nucleotide sequence represented by SEQ ID NOS: 116307, 116308, 116309, 116310, 116311, 116312, 116313, 116314, 116315, 116316, 116317 were selected from the ORFs having the nucleotide sequence represented by SEQ ID NO: 78343.

The probes having the nucleotide sequence represented by SEQ ID NOS: 116318, 116319, 116320, 116321, 116322, 116323, 116324, 116325, 116326, 116327, 116328 were selected from the ORFs having the nucleotide sequence represented by SEQ ID NO: 78344.

The probes having the nucleotide sequence represented by SEQ ID NOS: 116329, 116330, 116331, 116332, 116333, 116334, 116335, 116336, 116337, 116338, 116339 were selected from the ORFs having the nucleotide sequence represented by SEQ ID NO: 78345.

The probes having the nucleotide sequence represented by SEQ ID NOS: 116340, 116341, 116342, 116343, 116344, 116345, 116346, 116347, 116348, 116349, 116350 were selected from the ORFs having the nucleotide sequence represented by SEQ ID NO: 78346.

The probes having the nucleotide sequence represented by SEQ ID NOS: 116351, 116352, 116353, 116354, 116355, 116356, 116357, 116358, 116359, 116360, 116361 were selected from the ORFs having the nucleotide sequence represented by SEQ ID NO: 78347.

The probes having the nucleotide sequence represented by SEQ ID NOS: 116362, 116363, 116364, 116365, 116366, 116367, 116368, 116369, 116370, 116371, 116372 were selected from the ORFs having the nucleotide sequence represented by SEQ ID NO: 78348.

The probes having the nucleotide sequence represented by SEQ ID NOS: 116373, 116374, 116375, 116376, 116377, 116378, 116379, 116380, 116381, 116382, 116383 were selected from the ORFs having the nucleotide sequence represented by SEQ ID NO: 78349.

The probes having the nucleotide sequence represented by SEQ ID NOS: 116384, 116385, 116386, 116387, 116388, 116389, 116390, 116391, 116392, 116393, 116394 were selected from the ORFs having the nucleotide sequence represented by SEQ ID NO: 78350.

The probes having the nucleotide sequence represented by SEQ ID NOS: 116395, 116396, 116397, 116398, 116399, 116400, 116401, 116402, 116403, 116404, 116405 were selected from the ORFs having the nucleotide sequence represented by SEQ ID NO: 78351.

The probes having the nucleotide sequence represented by SEQ ID NOS: 116406, 116407, 116408, 116409, 116410, 116411, 116412, 116413, 116414, 116415, 116416 were selected from the ORFs having the nucleotide sequence represented by SEQ ID NO: 78352.

The probes having the nucleotide sequence represented by SEQ ID NOS: 116417, 116418, 116419, 116420, 116421, 116422, 116423, 116424, 116425, 116426, 116427 were selected from the ORFs having the nucleotide sequence represented by SEQ ID NO: 78353.

The probes having the nucleotide sequence represented by SEQ ID NOS: 116428, 116429, 116430, 116431, 116432, 116433, 116434, 116435, 116436, 116437, 116438 were selected from the ORFs having the nucleotide sequence represented by SEQ ID NO: 78354.

The probes having the nucleotide sequence represented by SEQ ID NOS: 116439, 116440, 116441, 116442, 116443, 116444, 116445, 116446, 116447, 116448, 116449 were selected from the ORFs having the nucleotide sequence represented by SEQ ID NO: 78355.

The probes having the nucleotide sequence represented by SEQ ID NOS: 116450, 116451, 116452, 116453, 116454, 116455, 116456, 116457, 116458, 116459, 116460 were selected from the ORFs having the nucleotide sequence represented by SEQ ID NO: 78356.

The probes having the nucleotide sequence represented by SEQ ID NOS: 116461, 116462, 116463, 116464, 116465, 116466, 116467, 116468, 116469, 116470, 116471 were selected from the ORFs having the nucleotide sequence represented by SEQ ID NO: 78357.

The probes having the nucleotide sequence represented by SEQ ID NOS: 116472, 116473, 116474, 116475, 116476, 116477, 116478, 116479, 116480, 116481, 116482 were selected from the ORFs having the nucleotide sequence represented by SEQ ID NO: 78358.

The probes having the nucleotide sequence represented by SEQ ID NOS: 116483, 116484, 116485, 116486, 116487, 116488, 116489, 116490, 116491, 116492, 116493 were selected from the ORFs having the nucleotide sequence represented by SEQ ID NO: 78359.

The probes having the nucleotide sequence represented by SEQ ID NOS: 116494, 116495, 116496, 116497, 116498, 116499, 116500, 116501, 116502, 116503, 116504 were selected from the ORFs having the nucleotide sequence represented by SEQ ID NO: 78360.

The probes having the nucleotide sequence represented by SEQ ID NOS: 116505, 116506, 116507, 116508, 116509, 116510, 116511, 116512, 116513, 116514, 116515 were selected from the ORFs having the nucleotide sequence represented by SEQ ID NO: 78361.

The probes having the nucleotide sequence represented by SEQ ID NOS: 116516, 116517, 116518, 116519, 116520, 116521, 116522, 116523, 116524, 116525, 116526 were selected from the ORFs having the nucleotide sequence represented by SEQ ID NO: 78362.

The probes having the nucleotide sequence represented by SEQ ID NOS: 116527, 116528, 116529, 116530, 116531, 116532, 116533, 116534, 116535, 116536, 116537 were selected from the ORFs having the nucleotide sequence represented by SEQ ID NO: 78363.

The probes having the nucleotide sequence represented by SEQ ID NOS: 116538, 116539, 116540, 116541, 116542, 116543, 116544, 116545, 116546, 116547, 116548 were selected from the ORFs having the nucleotide sequence represented by SEQ ID NO: 78364.

The probes having the nucleotide sequence represented by SEQ ID NOS: 116549, 116550, 116551, 116552, 116553, 116554, 116555, 116556, 116557, 116558, 116559 were selected from the ORFs having the nucleotide sequence represented by SEQ ID NO: 78365.

The probes having the nucleotide sequence represented by SEQ ID NOS: 116560, 116561, 116562, 116563, 116564, 116565, 116566, 116567, 116568, 116569, 116570 were selected from the ORFs having the nucleotide sequence represented by SEQ ID NO: 78366.

The probes having the nucleotide sequence represented by SEQ ID NOS: 116571, 116572, 116573, 116574, 116575, 116576, 116577, 116578, 116579, 116580, 116581 were selected from the ORFs having the nucleotide sequence represented by SEQ ID NO: 78367.

The probes having the nucleotide sequence represented by SEQ ID NOS: 116582, 116583, 116584, 116585, 116586, 116587, 116588, 116589, 116590, 116591, 116592 were selected from the ORFs having the nucleotide sequence represented by SEQ ID NO: 78368.

The probes having the nucleotide sequence represented by SEQ ID NOS: 116593, 116594, 116595, 116596, 116597, 116598, 116599, 116600, 116601, 116602, 116603 were selected from the ORFs having the nucleotide sequence represented by SEQ ID NO: 78369.

The probes having the nucleotide sequence represented by SEQ ID NOS: 116604, 116605, 116606, 116607, 116608, 116609, 116610, 116611, 116612, 116613, 116614 were selected from the ORFs having the nucleotide sequence represented by SEQ ID NO: 78370.

The probes having the nucleotide sequence represented by SEQ ID NOS: 116615, 116616, 116617, 116618, 116619, 116620, 116621, 116622, 116623, 116624 were selected from the ORFs having the nucleotide sequence represented by SEQ ID NO: 78371.

The probes having the nucleotide sequence represented by SEQ ID NOS: 116625, 116626, 116627, 116628, 116629, 116630, 116631, 116632, 116633, 116634, 116635 were selected from the ORFs having the nucleotide sequence represented by SEQ ID NO: 78372.

The probes having the nucleotide sequence represented by SEQ ID NOS: 116636, 116637, 116638, 116639, 116640, 116641, 116642, 116643, 116644, 116645, 116646 were selected from the ORFs having the nucleotide sequence represented by SEQ ID NO: 78373.

The probes having the nucleotide sequence represented by SEQ ID NOS: 116647, 116648, 116649, 116650, 116651, 116652, 116653, 116654, 116655, 116656, 116657 were selected from the ORFs having the nucleotide sequence represented by SEQ ID NO: 78374.

The probes having the nucleotide sequence represented by SEQ ID NOS: 116658, 116659, 116660, 116661, 116662, 116663, 116664, 116665, 116666, 116667, 116668 were selected from the ORFs having the nucleotide sequence represented by SEQ ID NO: 78375.

The probes having the nucleotide sequence represented by SEQ ID NOS: 116669, 116670, 116671, 116672, 116673, 116674, 116675, 116676, 116677, 116678, 116679 were selected from the ORFs having the nucleotide sequence represented by SEQ ID NO: 78376.

The probes having the nucleotide sequence represented by SEQ ID NOS: 116680, 116681, 116682, 116683, 116684, 116685, 116686, 116687, 116688, 116689, 116690 were selected from the ORFs having the nucleotide sequence represented by SEQ ID NO: 78377.

The probes having the nucleotide sequence represented by SEQ ID NOS: 116691, 116692, 116693, 116694, 116695, 116696, 116697, 116698, 116699, 116700, 116701 were selected from the ORFs having the nucleotide sequence represented by SEQ ID NO: 78378.

The probes having the nucleotide sequence represented by SEQ ID NOS: 116702, 116703, 116704, 116705, 116706, 116707, 116708, 116709, 116710, 116711, 116712 were selected from the ORFs having the nucleotide sequence represented by SEQ ID NO: 78379.

The probes having the nucleotide sequence represented by SEQ ID NOS: 116713, 116714, 116715, 116716, 116717, 116718, 116719, 116720, 116721, 116722, 116723 were selected from the ORFs having the nucleotide sequence represented by SEQ ID NO: 78380.

The probes having the nucleotide sequence represented by SEQ ID NOS: 116724, 116725, 116726, 116727, 116728, 116729, 116730, 116731, 116732, 116733, 116734 were selected from the ORFs having the nucleotide sequence represented by SEQ ID NO: 78381.

The probes having the nucleotide sequence represented by SEQ ID NOS: 116735, 116736, 116737, 116738, 116739, 116740, 116741, 116742, 116743, 116744, 116745 were selected from the ORFs having the nucleotide sequence represented by SEQ ID NO: 78382.

The probes having the nucleotide sequence represented by SEQ ID NOS: 116746, 116747, 116748, 116749, 116750, 116751, 116752, 116753, 116754, 116755, 116756 were selected from the ORFs having the nucleotide sequence represented by SEQ ID NO: 78383.

The probes having the nucleotide sequence represented by SEQ ID NOS: 116757, 116758, 116759, 116760, 116761, 116762, 116763, 116764, 116765, 116766, 116767 were selected from the ORFs having the nucleotide sequence represented by SEQ ID NO: 78384.

The probes having the nucleotide sequence represented by SEQ ID NOS: 116768, 116769, 116770, 116771, 116772, 116773, 116774, 116775, 116776, 116777, 116778 were selected from the ORFs having the nucleotide sequence represented by SEQ ID NO: 78385.

The probes having the nucleotide sequence represented by SEQ ID NOS: 116779, 116780, 116781, 116782, 116783, 116784, 116785, 116786, 116787, 116788, 116789 were selected from the ORFs having the nucleotide sequence represented by SEQ ID NO: 78386.

The probes having the nucleotide sequence represented by SEQ ID NOS: 116790, 116791, 116792, 116793, 116794, 116795, 116796, 116797, 116798, 116799, 116800 were selected from the ORFs having the nucleotide sequence represented by SEQ ID NO: 78387.

The probes having the nucleotide sequence represented by SEQ ID NOS: 116801, 116802, 116803, 116804, 116805, 116806, 116807, 116808, 116809, 116810, 116811 were selected from the ORFs having the nucleotide sequence represented by SEQ ID NO: 78388.

The probes having the nucleotide sequence represented by SEQ ID NOS: 116812, 116813, 116814, 116815, 116816, 116817, 116818, 116819, 116820, 116821, 116822 were selected from the ORFs having the nucleotide sequence represented by SEQ ID NO: 78389.

The probes having the nucleotide sequence represented by SEQ ID NOS: 116823, 116824, 116825, 116826, 116827, 116828, 116829, 116830, 116831, 116832, 116833 were selected from the ORFs having the nucleotide sequence represented by SEQ ID NO: 78390.

The probes having the nucleotide sequence represented by SEQ ID NOS: 116834, 116835, 116836, 116837, 116838, 116839, 116840, 116841, 116842, 116843, 116844 were selected from the ORFs having the nucleotide sequence represented by SEQ ID NO: 78391.

The probes having the nucleotide sequence represented by SEQ ID NOS: 116845, 116846, 116847, 116848, 116849, 116850, 116851, 116852, 116853, 116854, 116855 were selected from the ORFs having the nucleotide sequence represented by SEQ ID NO: 78392.

The probes having the nucleotide sequence represented by SEQ ID NOS: 116856, 116857, 116858, 116859, 116860, 116861, 116862, 116863, 116864, 116865, 116866 were selected from the ORFs having the nucleotide sequence represented by SEQ ID NO: 78393.

The probes having the nucleotide sequence represented by SEQ ID NOS: 116867, 116868, 116869, 116870, 116871, 116872, 116873, 116874, 116875, 116876, 116877 were selected from the ORFs having the nucleotide sequence represented by SEQ ID NO: 78394.

The probes having the nucleotide sequence represented by SEQ ID NOS: 116878, 116879, 116880, 116881, 116882, 116883, 116884, 116885, 116886, 116887, 116888 were selected from the ORFs having the nucleotide sequence represented by SEQ ID NO: 78395.

The probes having the nucleotide sequence represented by SEQ ID NOS: 116889, 116890, 116891, 116892, 116893, 116894, 116895, 116896, 116897, 116898, 116899 were selected from the ORFs having the nucleotide sequence represented by SEQ ID NO: 78396.

The probes having the nucleotide sequence represented by SEQ ID NOS: 116900, 116901, 116902, 116903, 116904, 116905, 116906, 116907, 116908, 116909, 116910 were selected from the ORFs having the nucleotide sequence represented by SEQ ID NO: 78397.

The probes having the nucleotide sequence represented by SEQ ID NOS: 116911, 116912, 116913, 116914, 116915, 116916, 116917, 116918, 116919, 116920, 116921 were selected from the ORFs having the nucleotide sequence represented by SEQ ID NO: 78398.

The probes having the nucleotide sequence represented by SEQ ID NOS: 116922, 116923, 116924, 116925, 116926, 116927, 116928, 116929, 116930, 116931, 116932 were selected from the ORFs having the nucleotide sequence represented by SEQ ID NO: 78399.

The probes having the nucleotide sequence represented by SEQ ID NOS: 116933, 116934, 116935, 116936, 116937, 116938, 116939, 116940, 116941, 116942, 116943 were selected from the ORFs having the nucleotide sequence represented by SEQ ID NO: 78400.

The probes having the nucleotide sequence represented by SEQ ID NOS: 116944, 116945, 116946, 116947, 116948, 116949, 116950, 116951, 116952, 116953, 116954 were selected from the ORFs having the nucleotide sequence represented by SEQ ID NO: 78401.

The probes having the nucleotide sequence represented by SEQ ID NOS: 116955, 116956, 116957, 116958, 116959, 116960, 116961, 116962, 116963, 116964, 116965 were selected from the ORFs having the nucleotide sequence represented by SEQ ID NO: 78402.

The probes having the nucleotide sequence represented by SEQ ID NOS: 116966, 116967, 116968, 116969, 116970, 116971, 116972, 116973, 116974, 116975, 116976 were selected from the ORFs having the nucleotide sequence represented by SEQ ID NO: 78403.

The probes having the nucleotide sequence represented by SEQ ID NOS: 116977, 116978, 116979, 116980, 116981, 116982, 116983, 116984, 116985, 116986, 116987 were selected from the ORFs having the nucleotide sequence represented by SEQ ID NO: 78404.

The probes having the nucleotide sequence represented by SEQ ID NOS: 116988, 116989, 116990, 116991, 116992, 116993, 116994, 116995, 116996, 116997, 116998 were selected from the ORFs having the nucleotide sequence represented by SEQ ID NO: 78405.

The probes having the nucleotide sequence represented by SEQ ID NOS: 116999, 117000, 117001, 117002, 117003, 117004, 117005, 117006, 117007, 117008, 117009 were selected from the ORFs having the nucleotide sequence represented by SEQ ID NO: 78406.

The probes having the nucleotide sequence represented by SEQ ID NOS: 117010, 117011, 117012, 117013, 117014, 117015, 117016, 117017, 117018, 117019, 117020 were selected from the ORFs having the nucleotide sequence represented by SEQ ID NO: 78407.

The probes having the nucleotide sequence represented by SEQ ID NOS: 117021, 117022, 117023, 117024, 117025, 117026, 117027, 117028, 117029, 117030, 117031 were selected from the ORFs having the nucleotide sequence represented by SEQ ID NO: 78408.

The probes having the nucleotide sequence represented by SEQ ID NOS: 117032, 117033, 117034, 117035, 117036, 117037, 117038, 117039, 117040, 117041, 117042 were selected from the ORFs having the nucleotide sequence represented by SEQ ID NO: 78409.

The probes having the nucleotide sequence represented by SEQ ID NOS: 117043, 117044, 117045, 117046, 117047, 117048, 117049, 117050, 117051, 117052, 117053 were selected from the ORFs having the nucleotide sequence represented by SEQ ID NO: 78410.

The probes having the nucleotide sequence represented by SEQ ID NOS: 117054, 117055, 117056, 117057, 117058, 117059, 117060, 117061, 117062, 117063, 117064 were selected from the ORFs having the nucleotide sequence represented by SEQ ID NO: 78411.

The probes having the nucleotide sequence represented by SEQ ID NOS: 117065, 117066, 117067, 117068, 117069, 117070, 117071, 117072, 117073, 117074, 117075 were selected from the ORFs having the nucleotide sequence represented by SEQ ID NO: 78412.

The probes having the nucleotide sequence represented by SEQ ID NOS: 117076, 117077, 117078, 117079, 117080, 117081, 117082, 117083, 117084, 117085, 117086 were selected from the ORFs having the nucleotide sequence represented by SEQ ID NO: 78413.

The probes having the nucleotide sequence represented by SEQ ID NOS: 117087, 117088, 117089, 117090, 117091, 117092, 117093, 117094, 117095, 117096, 117097 were selected from the ORFs having the nucleotide sequence represented by SEQ ID NO: 78414.

The probes having the nucleotide sequence represented by SEQ ID NOS: 117098, 117099, 117100, 117101, 117102, 117103, 117104, 117105, 117106, 117107, 117108 were selected from the ORFs having the nucleotide sequence represented by SEQ ID NO: 78415.

The probes having the nucleotide sequence represented by SEQ ID NOS: 117109, 117110, 117111, 117112, 117113, 117114, 117115, 117116, 117117, 117118, 117119 were selected from the ORFs having the nucleotide sequence represented by SEQ ID NO: 78416.

The probes having the nucleotide sequence represented by SEQ ID NOS: 117120, 117121, 117122, 117123, 117124, 117125, 117126, 117127, 117128, 117129, 117130 were selected from the ORFs having the nucleotide sequence represented by SEQ ID NO: 78417.

The probes having the nucleotide sequence represented by SEQ ID NOS: 117131, 117132, 117133, 117134, 117135, 117136, 117137, 117138, 117139, 117140, 117141 were selected from the ORFs having the nucleotide sequence represented by SEQ ID NO: 78418.

The probes having the nucleotide sequence represented by SEQ ID NOS: 117142, 117143, 117144, 117145, 117146, 117147, 117148, 117149, 117150, 117151, 117152 were selected from the ORFs having the nucleotide sequence represented by SEQ ID NO: 78419.

The probes having the nucleotide sequence represented by SEQ ID NOS: 117153, 117154, 117155, 117156, 117157, 117158, 117159, 117160, 117161, 117162, 117163 were selected from the ORFs having the nucleotide sequence represented by SEQ ID NO: 78420.

The probes having the nucleotide sequence represented by SEQ ID NOS: 117164, 117165, 117166, 117167, 117168, 117169, 117170, 117171, 117172, 117173, 117174 were selected from the ORFs having the nucleotide sequence represented by SEQ ID NO: 78421.

The probes having the nucleotide sequence represented by SEQ ID NOS: 117175, 117176, 117177, 117178, 117179, 117180, 117181, 117182, 117183, 117184, 117185 were selected from the ORFs having the nucleotide sequence represented by SEQ ID NO: 78422.

The probes having the nucleotide sequence represented by SEQ ID NOS: 117186, 117187, 117188, 117189, 117190, 117191, 117192, 117193, 117194, 117195, 117196 were selected from the ORFs having the nucleotide sequence represented by SEQ ID NO: 78423.

The probes having the nucleotide sequence represented by SEQ ID NOS: 117197, 117198, 117199, 117200, 117201, 117202, 117203, 117204, 117205, 117206, 117207 were selected from the ORFs having the nucleotide sequence represented by SEQ ID NO: 78424.

The probes having the nucleotide sequence represented by SEQ ID NOS: 117208, 117209, 117210, 117211, 117212, 117213, 117214, 117215, 117216, 117217, 117218 were selected from the ORFs having the nucleotide sequence represented by SEQ ID NO: 78425.

The probes having the nucleotide sequence represented by SEQ ID NOS: 117219, 117220, 117221, 117222, 117223, 117224, 117225, 117226, 117227, 117228, 117229 were selected from the ORFs having the nucleotide sequence represented by SEQ ID NO: 78426.

The probes having the nucleotide sequence represented by SEQ ID NOS: 117230, 117231, 117232, 117233, 117234, 117235, 117236, 117237, 117238, 117239, 117240 were selected from the ORFs having the nucleotide sequence represented by SEQ ID NO: 78427.

The probes having the nucleotide sequence represented by SEQ ID NOS: 117241, 117242, 117243, 117244, 117245, 117246, 117247, 117248, 117249, 117250, 117251 were selected from the ORFs having the nucleotide sequence represented by SEQ ID NO: 78428.

The probes having the nucleotide sequence represented by SEQ ID NOS: 117252, 117253, 117254, 117255, 117256, 117257, 117258, 117259, 117260, 117261, 117262 were selected from the ORFs having the nucleotide sequence represented by SEQ ID NO: 78429.

The probes having the nucleotide sequence represented by SEQ ID NOS: 117263, 117264, 117265, 117266, 117267, 117268, 117269, 117270, 117271, 117272, 117273 were selected from the ORFs having the nucleotide sequence represented by SEQ ID NO: 78430.

The probes having the nucleotide sequence represented by SEQ ID NOS: 117274, 117275, 117276, 117277, 117278, 117279, 117280, 117281, 117282, 117283, 117284 were selected from the ORFs having the nucleotide sequence represented by SEQ ID NO: 78431.

The probes having the nucleotide sequence represented by SEQ ID NOS: 117285, 117286, 117287, 117288, 117289, 117290, 117291, 117292, 117293, 117294, 117295 were selected from the ORFs having the nucleotide sequence represented by SEQ ID NO: 78432.

The probes having the nucleotide sequence represented by SEQ ID NOS: 117296, 117297, 117298, 117299, 117300, 117301, 117302, 117303, 117304, 117305, 117306 were selected from the ORFs having the nucleotide sequence represented by SEQ ID NO: 78433.

The probes having the nucleotide sequence represented by SEQ ID NOS: 117307, 117308, 117309, 117310, 117311, 117312, 117313, 117314, 117315, 117316, 117317 were selected from the ORFs having the nucleotide sequence represented by SEQ ID NO: 78434.

The probes having the nucleotide sequence represented by SEQ ID NOS: 117318, 117319, 117320, 117321, 117322, 117323, 117324, 117325, 117326, 117327, 117328 were selected from the ORFs having the nucleotide sequence represented by SEQ ID NO: 78435.

The probes having the nucleotide sequence represented by SEQ ID NOS: 117329, 117330, 117331, 117332, 117333, 117334, 117335, 117336, 117337, 117338, 117339 were selected from the ORFs having the nucleotide sequence represented by SEQ ID NO: 78436.

The probes having the nucleotide sequence represented by SEQ ID NOS: 117340, 117341, 117342, 117343, 117344, 117345, 117346, 117347, 117348, 117349, 117350 were selected from the ORFs having the nucleotide sequence represented by SEQ ID NO: 78437.

The probes having the nucleotide sequence represented by SEQ ID NOS: 117351, 117352, 117353, 117354, 117355, 117356, 117357, 117358, 117359, 117360, 117361, 117362, 117363, 117364, 117365, 117366, 117367, 117368, 117369, 117370, 117371, 117372 were selected from the ORFs having the nucleotide sequence represented by SEQ ID NO: 78438.

The probes having the nucleotide sequence represented by SEQ ID NOS: 117373, 117374, 117375, 117376, 117377, 117378, 117379, 117380, 117381, 117382, 117383 were selected from the ORFs having the nucleotide sequence represented by SEQ ID NO: 78439.

The probes having the nucleotide sequence represented by SEQ ID NOS: 117384, 117385, 117386, 117387, 117388, 117389, 117390, 117391, 117392, 117393, 117394 were selected from the ORFs having the nucleotide sequence represented by SEQ ID NO: 78440.

The probes having the nucleotide sequence represented by SEQ ID NOS: 117395, 117396, 117397, 117398, 117399, 117400, 117401, 117402, 117403, 117404, 117405 were selected from the ORFs having the nucleotide sequence represented by SEQ ID NO: 78441.

The probes having the nucleotide sequence represented by SEQ ID NOS: 117406, 117407, 117408, 117409, 117410, 117411, 117412, 117413, 117414, 117415, 117416 were selected from the ORFs having the nucleotide sequence represented by SEQ ID NO: 78442.

The probes having the nucleotide sequence represented by SEQ ID NOS: 117417, 117418, 117419, 117420, 117421, 117422, 117423, 117424, 117425, 117426, 117427 were selected from the ORFs having the nucleotide sequence represented by SEQ ID NO: 78443.

The probes having the nucleotide sequence represented by SEQ ID NOS: 117428, 117429, 117430, 117431, 117432, 117433, 117434, 117435, 117436, 117437, 117438 were selected from the ORFs having the nucleotide sequence represented by SEQ ID NO: 78444.

The probes having the nucleotide sequence represented by SEQ ID NOS: 117439, 117440, 117441, 117442, 117443, 117444, 117445, 117446, 117447, 117448, 117449 were selected from the ORFs having the nucleotide sequence represented by SEQ ID NO: 78445.

The probes having the nucleotide sequence represented by SEQ ID NOS: 117450, 117451, 117452, 117453, 117454, 117455, 117456, 117457, 117458, 117459, 117460 were selected from the ORFs having the nucleotide sequence represented by SEQ ID NO: 78446.

The probes having the nucleotide sequence represented by SEQ ID NOS: 117461, 117462, 117463, 117464, 117465, 117466, 117467, 117468, 117469, 117470, 117471 were selected from the ORFs having the nucleotide sequence represented by SEQ ID NO: 78447.

The probes having the nucleotide sequence represented by SEQ ID NOS: 117472, 117473, 117474, 117475, 117476, 117477, 117478, 117479, 117480, 117481, 117482 were selected from the ORFs having the nucleotide sequence represented by SEQ ID NO: 78448.

The probes having the nucleotide sequence represented by SEQ ID NOS: 117483, 117484, 117485, 117486, 117487, 117488, 117489, 117490, 117491, 117492, 117493 were selected from the ORFs having the nucleotide sequence represented by SEQ ID NO: 78449.

The probes having the nucleotide sequence represented by SEQ ID NOS: 117494, 117495, 117496, 117497, 117498, 117499, 117500, 117501, 117502, 117503, 117504 were selected from the ORFs having the nucleotide sequence represented by SEQ ID NO: 78450.

The probes having the nucleotide sequence represented by SEQ ID NOS: 117505, 117506, 117507, 117508, 117509, 117510, 117511, 117512, 117513, 117514, 117515 were selected from the ORFs having the nucleotide sequence represented by SEQ ID NO: 78451.

The probes having the nucleotide sequence represented by SEQ ID NOS: 117516, 117517, 117518, 117519, 117520, 117521, 117522, 117523, 117524, 117525, 117526 were selected from the ORFs having the nucleotide sequence represented by SEQ ID NO: 78452.

The probes having the nucleotide sequence represented by SEQ ID NOS: 117527, 117528, 117529, 117530, 117531, 117532, 117533, 117534, 117535, 117536, 117537 were selected from the ORFs having the nucleotide sequence represented by SEQ ID NO: 78453.

The probes having the nucleotide sequence represented by SEQ ID NOS: 117538, 117539, 117540, 117541, 117542, 117543, 117544, 117545, 117546, 117547, 117548 were selected from the ORFs having the nucleotide sequence represented by SEQ ID NO: 78454.

The probes having the nucleotide sequence represented by SEQ ID NOS: 117549, 117550, 117551, 117552, 117553, 117554, 117555, 117556, 117557, 117558, 117559 were selected from the ORFs having the nucleotide sequence represented by SEQ ID NO: 78455.

The probes having the nucleotide sequence represented by SEQ ID NOS: 117560, 117561, 117562, 117563, 117564, 117565, 117566, 117567, 117568, 117569, 117570 were selected from the ORFs having the nucleotide sequence represented by SEQ ID NO: 78456.

The probes having the nucleotide sequence represented by SEQ ID NOS: 117571, 117572, 117573, 117574, 117575, 117576, 117577, 117578, 117579, 117580, 117581 were selected from the ORFs having the nucleotide sequence represented by SEQ ID NO: 78457.

The probes having the nucleotide sequence represented by SEQ ID NOS: 117582, 117583, 117584, 117585, 117586, 117587, 117588, 117589, 117590, 117591, 117592 were selected from the ORFs having the nucleotide sequence represented by SEQ ID NO: 78458.

The probes having the nucleotide sequence represented by SEQ ID NOS: 117593, 117594, 117595, 117596, 117597, 117598, 117599, 117600, 117601, 117602, 117603 were selected from the ORFs having the nucleotide sequence represented by SEQ ID NO: 78459.

The probes having the nucleotide sequence represented by SEQ ID NOS: 117604, 117605, 117606, 117607, 117608, 117609, 117610, 117611, 117612, 117613, 117614 were selected from the ORFs having the nucleotide sequence represented by SEQ ID NO: 78460.

The probes having the nucleotide sequence represented by SEQ ID NOS: 117615, 117616, 117617, 117618, 117619, 117620, 117621, 117622, 117623, 117624, 117625 were selected from the ORFs having the nucleotide sequence represented by SEQ ID NO: 78461.

The probes having the nucleotide sequence represented by SEQ ID NOS: 117626, 117627, 117628, 117629, 117630, 117631, 117632, 117633, 117634, 117635, 117636 were selected from the ORFs having the nucleotide sequence represented by SEQ ID NO: 78462.

The probes having the nucleotide sequence represented by SEQ ID NOS: 117637, 117638, 117639, 117640, 117641, 117642, 117643, 117644, 117645, 117646, 117647 were selected from the ORFs having the nucleotide sequence represented by SEQ ID NO: 78463.

The probes having the nucleotide sequence represented by SEQ ID NOS: 117648, 117649, 117650, 117651, 117652, 117653, 117654, 117655, 117656, 117657, 117658 were selected from the ORFs having the nucleotide sequence represented by SEQ ID NO: 78464.

The probes having the nucleotide sequence represented by SEQ ID NOS: 117659, 117660, 117661, 117662, 117663, 117664, 117665, 117666, 117667, 117668, 117669 were selected from the ORFs having the nucleotide sequence represented by SEQ ID NO: 78465.

The probes having the nucleotide sequence represented by SEQ ID NOS: 117670, 117671, 117672, 117673, 117674, 117675, 117676, 117677, 117678, 117679, 117680 were selected from the ORFs having the nucleotide sequence represented by SEQ ID NO: 78466.

The probes having the nucleotide sequence represented by SEQ ID NOS: 117681, 117682, 117683, 117684, 117685, 117686, 117687, 117688, 117689, 117690, 117691 were selected from the ORFs having the nucleotide sequence represented by SEQ ID NO: 78467.

The probes having the nucleotide sequence represented by SEQ ID NOS: 117692, 117693, 117694, 117695, 117696, 117697, 117698, 117699, 117700, 117701, 117702 were selected from the ORFs having the nucleotide sequence represented by SEQ ID NO: 78468.

The probes having the nucleotide sequence represented by SEQ ID NOS: 117703, 117704, 117705, 117706, 117707, 117708, 117709, 117710, 117711, 117712, 117713 were selected from the ORFs having the nucleotide sequence represented by SEQ ID NO: 78469.

The probes having the nucleotide sequence represented by SEQ ID NOS: 117714, 117715, 117716, 117717, 117718, 117719, 117720, 117721, 117722, 117723, 117724 were selected from the ORFs having the nucleotide sequence represented by SEQ ID NO: 78470.

The probes having the nucleotide sequence represented by SEQ ID NOS: 117725, 117726, 117727, 117728, 117729, 117730, 117731, 117732, 117733, 117734, 117735 were selected from the ORFs having the nucleotide sequence represented by SEQ ID NO: 78471.

The probes having the nucleotide sequence represented by SEQ ID NOS: 117736, 117737, 117738, 117739, 117740, 117741, 117742, 117743, 117744, 117745, 117746 were selected from the ORFs having the nucleotide sequence represented by SEQ ID NO: 78472.

The probes having the nucleotide sequence represented by SEQ ID NOS: 117747, 117748, 117749, 117750, 117751, 117752, 117753, 117754, 117755, 117756, 117757 were selected from the ORFs having the nucleotide sequence represented by SEQ ID NO: 78473.

The probes having the nucleotide sequence represented by SEQ ID NOS: 117758, 117759, 117760, 117761, 117762, 117763, 117764, 117765, 117766, 117767, 117768 were selected from the ORFs having the nucleotide sequence represented by SEQ ID NO: 78474.

The probes having the nucleotide sequence represented by SEQ ID NOS: 117769, 117770, 117771, 117772, 117773, 117774, 117775, 117776, 117777, 117778, 117779 were selected from the ORFs having the nucleotide sequence represented by SEQ ID NO: 78475.

The probes having the nucleotide sequence represented by SEQ ID NOS: 117780, 117781, 117782, 117783, 117784, 117785, 117786, 117787, 117788, 117789, 117790 were selected from the ORFs having the nucleotide sequence represented by SEQ ID NO: 78476.

The probes having the nucleotide sequence represented by SEQ ID NOS: 117791, 117792, 117793, 117794, 117795, 117796, 117797, 117798, 117799, 117800, 117801 were selected from the ORFs having the nucleotide sequence represented by SEQ ID NO: 78477.

The probes having the nucleotide sequence represented by SEQ ID NOS: 117802, 117803, 117804, 117805, 117806, 117807, 117808, 117809, 117810, 117811, 117812 were selected from the ORFs having the nucleotide sequence represented by SEQ ID NO: 78478.

The probes having the nucleotide sequence represented by SEQ ID NOS: 117813, 117814, 117815, 117816, 117817, 117818, 117819, 117820, 117821, 117822, 117823 were selected from the ORFs having the nucleotide sequence represented by SEQ ID NO: 78479.

The probes having the nucleotide sequence represented by SEQ ID NOS: 117824, 117825, 117826, 117827, 117828, 117829, 117830, 117831, 117832, 117833, 117834 were selected from the ORFs having the nucleotide sequence represented by SEQ ID NO: 78480.

The probes having the nucleotide sequence represented by SEQ ID NOS: 117835, 117836, 117837, 117838, 117839, 117840, 117841, 117842, 117843, 117844, 117845 were selected from the ORFs having the nucleotide sequence represented by SEQ ID NO: 78481.

The probes having the nucleotide sequence represented by SEQ ID NOS: 117846, 117847, 117848, 117849, 117850, 117851, 117852, 117853, 117854, 117855, 117856 were selected from the ORFs having the nucleotide sequence represented by SEQ ID NO: 78482.

The probes having the nucleotide sequence represented by SEQ ID NOS: 117857, 117858, 117859, 117860, 117861, 117862, 117863, 117864, 117865, 117866, 117867 were selected from the ORFs having the nucleotide sequence represented by SEQ ID NO: 78483.

The probes having the nucleotide sequence represented by SEQ ID NOS: 117868, 117869, 117870, 117871, 117872, 117873, 117874, 117875, 117876, 117877, 117878 were selected from the ORFs having the nucleotide sequence represented by SEQ ID NO: 78484.

The probes having the nucleotide sequence represented by SEQ ID NOS: 117879, 117880, 117881, 117882, 117883, 117884, 117885, 117886, 117887, 117888, 117889 were selected from the ORFs having the nucleotide sequence represented by SEQ ID NO: 78485.

The probes having the nucleotide sequence represented by SEQ ID NOS: 117890, 117891, 117892, 117893, 117894, 117895, 117896, 117897, 117898, 117899, 117900 were selected from the ORFs having the nucleotide sequence represented by SEQ ID NO: 78486.

The probes having the nucleotide sequence represented by SEQ ID NOS: 117901, 117902, 117903, 117904, 117905, 117906, 117907, 117908, 117909, 117910, 117911 were selected from the ORFs having the nucleotide sequence represented by SEQ ID NO: 78487.

The probes having the nucleotide sequence represented by SEQ ID NOS: 117912, 117913, 117914, 117915, 117916, 117917, 117918, 117919, 117920, 117921, 117922 were selected from the ORFs having the nucleotide sequence represented by SEQ ID NO: 78488.

The probes having the nucleotide sequence represented by SEQ ID NOS: 117923, 117924, 117925, 117926, 117927, 117928, 117929, 117930, 117931, 117932, 117933 were selected from the ORFs having the nucleotide sequence represented by SEQ ID NO: 78489.

The probes having the nucleotide sequence represented by SEQ ID NOS: 117934, 117935, 117936, 117937, 117938, 117939, 117940, 117941, 117942, 117943, 117944 were selected from the ORFs having the nucleotide sequence represented by SEQ ID NO: 78490.

The probes having the nucleotide sequence represented by SEQ ID NOS: 117945, 117946, 117947, 117948, 117949, 117950, 117951, 117952, 117953, 117954, 117955 were selected from the ORFs having the nucleotide sequence represented by SEQ ID NO: 78491.

The probes having the nucleotide sequence represented by SEQ ID NOS: 117956, 117957, 117958, 117959, 117960, 117961, 117962, 117963, 117964, 117965, 117966 were selected from the ORFs having the nucleotide sequence represented by SEQ ID NO: 78492.

The probes having the nucleotide sequence represented by SEQ ID NOS: 117967, 117968, 117969, 117970, 117971, 117972, 117973, 117974, 117975, 117976, 117977 were selected from the ORFs having the nucleotide sequence represented by SEQ ID NO: 78493.

The probes having the nucleotide sequence represented by SEQ ID NOS: 117978, 117979, 117980, 117981, 117982, 117983, 117984, 117985, 117986, 117987, 117988 were selected from the ORFs having the nucleotide sequence represented by SEQ ID NO: 78494.

The probes having the nucleotide sequence represented by SEQ ID NOS: 117989, 117990, 117991, 117992, 117993, 117994, 117995, 117996, 117997, 117998, 117999 were selected from the ORFs having the nucleotide sequence represented by SEQ ID NO: 78495.

The probes having the nucleotide sequence represented by SEQ ID NOS: 118000, 118001, 118002, 118003, 118004, 118005, 118006, 118007, 118008, 118009, 118010 were selected from the ORFs having the nucleotide sequence represented by SEQ ID NO: 78496.

The probes having the nucleotide sequence represented by SEQ ID NOS: 118011, 118012, 118013, 118014, 118015, 118016, 118017, 118018, 118019, 118020, 118021 were selected from the ORFs having the nucleotide sequence represented by SEQ ID NO: 78497.

The probes having the nucleotide sequence represented by SEQ ID NOS: 118022, 118023, 118024, 118025, 118026, 118027, 118028, 118029, 118030, 118031, 118032 were selected from the ORFs having the nucleotide sequence represented by SEQ ID NO: 78498.

The probes having the nucleotide sequence represented by SEQ ID NOS: 118033, 118034, 118035, 118036, 118037, 118038, 118039, 118040, 118041, 118042, 118043 were selected from the ORFs having the nucleotide sequence represented by SEQ ID NO: 78499.

The probes having the nucleotide sequence represented by SEQ ID NOS: 118044, 118045, 118046, 118047, 118048, 118049, 118050, 118051, 118052, 118053, 118054 were selected from the ORFs having the nucleotide sequence represented by SEQ ID NO: 78500.

The probes having the nucleotide sequence represented by SEQ ID NOS: 118055, 118056, 118057, 118058, 118059, 118060, 118061, 118062, 118063, 118064, 118065 were selected from the ORFs having the nucleotide sequence represented by SEQ ID NO: 78501.

The probes having the nucleotide sequence represented by SEQ ID NOS: 118066, 118067, 118068, 118069, 118070, 118071, 118072, 118073, 118074, 118075, 118076 were selected from the ORFs having the nucleotide sequence represented by SEQ ID NO: 78502.

The probes having the nucleotide sequence represented by SEQ ID NOS: 118077, 118078, 118079, 118080, 118081, 118082, 118083, 118084, 118085, 118086, 118087 were selected from the ORFs having the nucleotide sequence represented by SEQ ID NO: 78503.

The probes having the nucleotide sequence represented by SEQ ID NOS: 118088, 118089, 118090, 118091, 118092, 118093, 118094, 118095, 118096, 118097, 118098 were selected from the ORFs having the nucleotide sequence represented by SEQ ID NO: 78504.

The probes having the nucleotide sequence represented by SEQ ID NOS: 118099, 118100, 118101, 118102, 118103, 118104, 118105, 118106, 118107, 118108, 118109 were selected from the ORFs having the nucleotide sequence represented by SEQ ID NO: 78505.

The probes having the nucleotide sequence represented by SEQ ID NOS: 118110, 118111, 118112, 118113, 118114, 118115, 118116, 118117, 118118, 118119, 118120 were selected from the ORFs having the nucleotide sequence represented by SEQ ID NO: 78506.

The probes having the nucleotide sequence represented by SEQ ID NOS: 118121, 118122, 118123, 118124, 118125, 118126, 118127, 118128, 118129, 118130, 118131 were selected from the ORFs having the nucleotide sequence represented by SEQ ID NO: 78507.

The probes having the nucleotide sequence represented by SEQ ID NOS: 118132, 118133, 118134, 118135, 118136, 118137, 118138, 118139, 118140, 118141, 118142 were selected from the ORFs having the nucleotide sequence represented by SEQ ID NO: 78508.

The probes having the nucleotide sequence represented by SEQ ID NOS: 118143, 118144, 118145, 118146, 118147, 118148, 118149, 118150, 118151, 118152, 118153 were selected from the ORFs having the nucleotide sequence represented by SEQ ID NO: 78509.

The probes having the nucleotide sequence represented by SEQ ID NOS: 118154, 118155, 118156, 118157, 118158, 118159, 118160, 118161, 118162, 118163, 118164 were selected from the ORFs having the nucleotide sequence represented by SEQ ID NO: 78510.

The probes having the nucleotide sequence represented by SEQ ID NOS: 118165, 118166, 118167, 118168, 118169, 118170, 118171, 118172, 118173, 118174, 118175 were selected from the ORFs having the nucleotide sequence represented by SEQ ID NO: 78511.

The probes having the nucleotide sequence represented by SEQ ID NOS: 118176, 118177, 118178, 118179, 118180, 118181, 118182, 118183, 118184, 118185, 118186 were selected from the ORFs having the nucleotide sequence represented by SEQ ID NO: 78512.

The probes having the nucleotide sequence represented by SEQ ID NOS: 118187, 118188, 118189, 118190, 118191, 118192, 118193, 118194, 118195, 118196, 118197 were selected from the ORFs having the nucleotide sequence represented by SEQ ID NO: 78513.

The probes having the nucleotide sequence represented by SEQ ID NOS: 118198, 118199, 118200, 118201, 118202, 118203, 118204, 118205, 118206, 118207, 118208 were selected from the ORFs having the nucleotide sequence represented by SEQ ID NO: 78514.

The probes having the nucleotide sequence represented by SEQ ID NOS: 118209, 118210, 118211, 118212, 118213, 118214, 118215, 118216, 118217, 118218, 118219 were selected from the ORFs having the nucleotide sequence represented by SEQ ID NO: 78515.

The probes having the nucleotide sequence represented by SEQ ID NOS: 118220, 118221, 118222, 118223, 118224, 118225, 118226, 118227, 118228, 118229, 118230 were selected from the ORFs having the nucleotide sequence represented by SEQ ID NO: 78516.

The probes having the nucleotide sequence represented by SEQ ID NOS: 118231, 118232, 118233, 118234, 118235, 118236, 118237, 118238, 118239, 118240, 118241, 118242, 118243, 118244, 118245, 118246, 118247, 118248, 118249, 118250, 118251, 118252 were selected from the ORFs having the nucleotide sequence represented by SEQ ID NO: 78517.

The probes having the nucleotide sequence represented by SEQ ID NOS: 118253, 118254, 118255, 118256, 118257, 118258, 118259, 118260, 118261, 118262, 118263, 118264, 118265, 118266, 118267, 118268, 118269, 118270, 118271, 118272, 118273, 118274 were selected from the ORFs having the nucleotide sequence represented by SEQ ID NO: 78518.

The probes having the nucleotide sequence represented by SEQ ID NOS: 118275, 118276, 118277, 118278, 118279, 118280, 118281, 118282, 118283, 118284, 118285 were selected from the ORFs having the nucleotide sequence represented by SEQ ID NO: 78519.

The probes having the nucleotide sequence represented by SEQ ID NOS: 118286, 118287, 118288, 118289, 118290, 118291, 118292, 118293, 118294, 118295, 118296 were selected from the ORFs having the nucleotide sequence represented by SEQ ID NO: 78520.

The probes having the nucleotide sequence represented by SEQ ID NOS: 118297, 118298, 118299, 118300, 118301, 118302, 118303, 118304, 118305, 118306, 118307 were selected from the ORFs having the nucleotide sequence represented by SEQ ID NO: 78521.

The probes having the nucleotide sequence represented by SEQ ID NOS: 118308, 118309, 118310, 118311, 118312, 118313, 118314, 118315, 118316, 118317, 118318 were selected from the ORFs having the nucleotide sequence represented by SEQ ID NO: 78522.

The probes having the nucleotide sequence represented by SEQ ID NOS: 118319, 118320, 118321, 118322, 118323, 118324, 118325, 118326, 118327, 118328, 118329 were selected from the ORFs having the nucleotide sequence represented by SEQ ID NO: 78523.

The probes having the nucleotide sequence represented by SEQ ID NOS: 118330, 118331, 118332, 118333, 118334, 118335, 118336, 118337, 118338, 118339, 118340 were selected from the ORFs having the nucleotide sequence represented by SEQ ID NO: 78524.

The probes having the nucleotide sequence represented by SEQ ID NOS: 118341, 118342, 118343, 118344, 118345, 118346, 118347, 118348, 118349, 118350, 118351 were selected from the ORFs having the nucleotide sequence represented by SEQ ID NO: 78525.

The probes having the nucleotide sequence represented by SEQ ID NOS: 118352, 118353, 118354, 118355, 118356, 118357, 118358, 118359, 118360, 118361, 118362 were selected from the ORFs having the nucleotide sequence represented by SEQ ID NO: 78526.

The probes having the nucleotide sequence represented by SEQ ID NOS: 118363, 118364, 118365, 118366, 118367, 118368, 118369, 118370, 118371, 118372, 118373 were selected from the ORFs having the nucleotide sequence represented by SEQ ID NO: 78527.

The probes having the nucleotide sequence represented by SEQ ID NOS: 118374, 118375, 118376, 118377, 118378, 118379, 118380, 118381, 118382, 118383, 118384 were selected from the ORFs having the nucleotide sequence represented by SEQ ID NO: 78528.

The probes having the nucleotide sequence represented by SEQ ID NOS: 118385, 118386, 118387, 118388, 118389, 118390, 118391, 118392, 118393, 118394, 118395 were selected from the ORFs having the nucleotide sequence represented by SEQ ID NO: 78529.

The probes having the nucleotide sequence represented by SEQ ID NOS: 118396, 118397, 118398, 118399, 118400, 118401, 118402, 118403, 118404, 118405, 118406 were selected from the ORFs having the nucleotide sequence represented by SEQ ID NO: 78530.

The probes having the nucleotide sequence represented by SEQ ID NOS: 118407, 118408, 118409, 118410, 118411, 118412, 118413, 118414, 118415, 118416, 118417 were selected from the ORFs having the nucleotide sequence represented by SEQ ID NO: 78531.

The probes having the nucleotide sequence represented by SEQ ID NOS: 118418, 118419, 118420, 118421, 118422, 118423, 118424, 118425, 118426, 118427, 118428 were selected from the ORFs having the nucleotide sequence represented by SEQ ID NO: 78532.

The probes having the nucleotide sequence represented by SEQ ID NOS: 118429, 118430, 118431, 118432, 118433, 118434, 118435, 118436, 118437, 118438, 118439 were selected from the ORFs having the nucleotide sequence represented by SEQ ID NO: 78533.

The probes having the nucleotide sequence represented by SEQ ID NOS: 118440, 118441, 118442, 118443, 118444, 118445, 118446, 118447, 118448, 118449, 118450 were selected from the ORFs having the nucleotide sequence represented by SEQ ID NO: 78534.

The probes having the nucleotide sequence represented by SEQ ID NOS: 118451, 118452, 118453, 118454, 118455, 118456, 118457, 118458, 118459, 118460, 118461 were selected from the ORFs having the nucleotide sequence represented by SEQ ID NO: 78535.

The probes having the nucleotide sequence represented by SEQ ID NOS: 118462, 118463, 118464, 118465, 118466, 118467, 118468, 118469, 118470, 118471, 118472 were selected from the ORFs having the nucleotide sequence represented by SEQ ID NO: 78536.

The probes having the nucleotide sequence represented by SEQ ID NOS: 118473, 118474, 118475, 118476, 118477, 118478, 118479, 118480, 118481, 118482, 118483 were selected from the ORFs having the nucleotide sequence represented by SEQ ID NO: 78537.

The probes having the nucleotide sequence represented by SEQ ID NOS: 118484, 118485, 118486, 118487, 118488, 118489, 118490, 118491, 118492, 118493, 118494 were selected from the ORFs having the nucleotide sequence represented by SEQ ID NO: 78538.

The probes having the nucleotide sequence represented by SEQ ID NOS: 118495, 118496, 118497, 118498, 118499, 118500, 118501, 118502, 118503, 118504, 118505 were selected from the ORFs having the nucleotide sequence represented by SEQ ID NO: 78539.

The probes having the nucleotide sequence represented by SEQ ID NOS: 118506, 118507, 118508, 118509, 118510, 118511, 118512, 118513, 118514, 118515, 118516 were selected from the ORFs having the nucleotide sequence represented by SEQ ID NO: 78540.

The probes having the nucleotide sequence represented by SEQ ID NOS: 118517, 118518, 118519, 118520, 118521, 118522, 118523, 118524, 118525, 118526, 118527 were selected from the ORFs having the nucleotide sequence represented by SEQ ID NO: 78541.

The probes having the nucleotide sequence represented by SEQ ID NOS: 118528, 118529, 118530, 118531, 118532, 118533, 118534, 118535, 118536, 118537, 118538 were selected from the ORFs having the nucleotide sequence represented by SEQ ID NO: 78542.

The probes having the nucleotide sequence represented by SEQ ID NOS: 118539, 118540, 118541, 118542, 118543, 118544, 118545, 118546, 118547, 118548, 118549 were selected from the ORFs having the nucleotide sequence represented by SEQ ID NO: 78543.

The probes having the nucleotide sequence represented by SEQ ID NOS: 118550, 118551, 118552, 118553, 118554, 118555, 118556, 118557, 118558, 118559, 118560 were selected from the ORFs having the nucleotide sequence represented by SEQ ID NO: 78544.

The probes having the nucleotide sequence represented by SEQ ID NOS: 118561, 118562, 118563, 118564, 118565, 118566, 118567, 118568, 118569, 118570, 118571 were selected from the ORFs having the nucleotide sequence represented by SEQ ID NO: 78545.

The probes having the nucleotide sequence represented by SEQ ID NOS: 118572, 118573, 118574, 118575, 118576, 118577, 118578, 118579, 118580, 118581, 118582 were selected from the ORFs having the nucleotide sequence represented by SEQ ID NO: 78546.

The probes having the nucleotide sequence represented by SEQ ID NOS: 118583, 118584, 118585, 118586, 118587, 118588, 118589, 118590, 118591, 118592, 118593 were selected from the ORFs having the nucleotide sequence represented by SEQ ID NO: 78547.

The probes having the nucleotide sequence represented by SEQ ID NOS: 118594, 118595, 118596, 118597, 118598, 118599, 118600, 118601, 118602, 118603, 118604 were selected from the ORFs having the nucleotide sequence represented by SEQ ID NO: 78548.

The probes having the nucleotide sequence represented by SEQ ID NOS: 118605, 118606, 118607, 118608, 118609, 118610, 118611, 118612, 118613, 118614, 118615 were selected from the ORFs having the nucleotide sequence represented by SEQ ID NO: 78549.

The probes having the nucleotide sequence represented by SEQ ID NOS: 118616, 118617, 118618, 118619, 118620, 118621, 118622, 118623, 118624, 118625, 118626 were selected from the ORFs having the nucleotide sequence represented by SEQ ID NO: 78550.

The probes having the nucleotide sequence represented by SEQ ID NOS: 118627, 118628, 118629, 118630, 118631, 118632, 118633, 118634, 118635, 118636, 118637 were selected from the ORFs having the nucleotide sequence represented by SEQ ID NO: 78551.

The probes having the nucleotide sequence represented by SEQ ID NOS: 118638, 118639, 118640, 118641, 118642, 118643, 118644, 118645, 118646, 118647, 118648 were selected from the ORFs having the nucleotide sequence represented by SEQ ID NO: 78552.

The probes having the nucleotide sequence represented by SEQ ID NOS: 118649, 118650, 118651, 118652, 118653, 118654, 118655, 118656, 118657, 118658, 118659 were selected from the ORFs having the nucleotide sequence represented by SEQ ID NO: 78553.

The probes having the nucleotide sequence represented by SEQ ID NOS: 118660, 118661, 118662, 118663, 118664, 118665, 118666, 118667, 118668, 118669, 118670 were selected from the ORFs having the nucleotide sequence represented by SEQ ID NO: 78554.

The probes having the nucleotide sequence represented by SEQ ID NOS: 118671, 118672, 118673, 118674, 118675, 118676, 118677, 118678, 118679, 118680, 118681 were selected from the ORFs having the nucleotide sequence represented by SEQ ID NO: 78555.

The probes having the nucleotide sequence represented by SEQ ID NOS: 118682, 118683, 118684, 118685, 118686, 118687, 118688, 118689, 118690, 118691, 118692 were selected from the ORFs having the nucleotide sequence represented by SEQ ID NO: 78556.

The probes having the nucleotide sequence represented by SEQ ID NOS: 118693, 118694, 118695, 118696, 118697, 118698, 118699, 118700, 118701, 118702, 118703 were selected from the ORFs having the nucleotide sequence represented by SEQ ID NO: 78557.

The probes having the nucleotide sequence represented by SEQ ID NOS: 118704, 118705, 118706, 118707, 118708, 118709, 118710, 118711, 118712, 118713, 118714 were selected from the ORFs having the nucleotide sequence represented by SEQ ID NO: 78558.

The probes having the nucleotide sequence represented by SEQ ID NOS: 118715, 118716, 118717, 118718, 118719, 118720, 118721, 118722, 118723, 118724, 118725 were selected from the ORFs having the nucleotide sequence represented by SEQ ID NO: 78559.

The probes having the nucleotide sequence represented by SEQ ID NOS: 118726, 118727, 118728, 118729, 118730, 118731, 118732, 118733, 118734, 118735, 118736 were selected from the ORFs having the nucleotide sequence represented by SEQ ID NO: 78560.

The probes having the nucleotide sequence represented by SEQ ID NOS: 118737, 118738, 118739, 118740, 118741, 118742, 118743, 118744, 118745, 118746, 118747 were selected from the ORFs having the nucleotide sequence represented by SEQ ID NO: 78561.

The probes having the nucleotide sequence represented by SEQ ID NOS: 118748, 118749, 118750, 118751, 118752, 118753, 118754, 118755, 118756, 118757, 118758 were selected from the ORFs having the nucleotide sequence represented by SEQ ID NO: 78562.

The probes having the nucleotide sequence represented by SEQ ID NOS: 118759, 118760, 118761, 118762, 118763, 118764, 118765, 118766, 118767, 118768, 118769 were selected from the ORFs having the nucleotide sequence represented by SEQ ID NO: 78563.

The probes having the nucleotide sequence represented by SEQ ID NOS: 118770, 118771, 118772, 118773, 118774, 118775, 118776, 118777, 118778, 118779, 118780 were selected from the ORFs having the nucleotide sequence represented by SEQ ID NO: 78564.

The probes having the nucleotide sequence represented by SEQ ID NOS: 118781, 118782, 118783, 118784, 118785, 118786, 118787, 118788, 118789, 118790, 118791 were selected from the ORFs having the nucleotide sequence represented by SEQ ID NO: 78565.

The probes having the nucleotide sequence represented by SEQ ID NOS: 118792, 118793, 118794, 118795, 118796, 118797, 118798, 118799, 118800, 118801, 118802 were selected from the ORFs having the nucleotide sequence represented by SEQ ID NO: 78566.

The probes having the nucleotide sequence represented by SEQ ID NOS: 118803, 118804, 118805, 118806, 118807, 118808, 118809, 118810, 118811, 118812, 118813 were selected from the ORFs having the nucleotide sequence represented by SEQ ID NO: 78567.

The probes having the nucleotide sequence represented by SEQ ID NOS: 118814, 118815, 118816, 118817, 118818, 118819, 118820, 118821, 118822, 118823, 118824 were selected from the ORFs having the nucleotide sequence represented by SEQ ID NO: 78568.

The probes having the nucleotide sequence represented by SEQ ID NOS: 118825, 118826, 118827, 118828, 118829, 118830, 118831, 118832, 118833, 118834, 118835 were selected from the ORFs having the nucleotide sequence represented by SEQ ID NO: 78569.

The probes having the nucleotide sequence represented by SEQ ID NOS: 118836, 118837, 118838, 118839, 118840, 118841, 118842, 118843, 118844, 118845, 118846 were selected from the ORFs having the nucleotide sequence represented by SEQ ID NO: 78570.

The probes having the nucleotide sequence represented by SEQ ID NOS: 118847, 118848, 118849, 118850, 118851, 118852, 118853, 118854, 118855, 118856, 118857 were selected from the ORFs having the nucleotide sequence represented by SEQ ID NO: 78571.

The probes having the nucleotide sequence represented by SEQ ID NOS: 118858, 118859, 118860, 118861, 118862, 118863, 118864, 118865, 118866, 118867, 118868 were selected from the ORFs having the nucleotide sequence represented by SEQ ID NO: 78572.

The probes having the nucleotide sequence represented by SEQ ID NOS: 118869, 118870, 118871, 118872, 118873, 118874, 118875, 118876, 118877, 118878, 118879 were selected from the ORFs having the nucleotide sequence represented by SEQ ID NO: 78573.

The probes having the nucleotide sequence represented by SEQ ID NOS: 118880, 118881, 118882, 118883, 118884, 118885, 118886, 118887, 118888, 118889, 118890 were selected from the ORFs having the nucleotide sequence represented by SEQ ID NO: 78574.

The probes having the nucleotide sequence represented by SEQ ID NOS: 118891, 118892, 118893, 118894, 118895, 118896, 118897, 118898, 118899, 118900, 118901 were selected from the ORFs having the nucleotide sequence represented by SEQ ID NO: 78575.

The probes having the nucleotide sequence represented by SEQ ID NOS: 118902, 118903, 118904, 118905, 118906, 118907, 118908, 118909, 118910, 118911, 118912 were selected from the ORFs having the nucleotide sequence represented by SEQ ID NO: 78576.

The probes having the nucleotide sequence represented by SEQ ID NOS: 118913, 118914, 118915, 118916, 118917, 118918, 118919, 118920, 118921, 118922, 118923 were selected from the ORFs having the nucleotide sequence represented by SEQ ID NO: 78577.

The probes having the nucleotide sequence represented by SEQ ID NOS: 118924, 118925, 118926, 118927, 118928, 118929, 118930, 118931, 118932, 118933, 118934 were selected from the ORFs having the nucleotide sequence represented by SEQ ID NO: 78578.

The probes having the nucleotide sequence represented by SEQ ID NOS: 118935, 118936, 118937, 118938, 118939, 118940, 118941, 118942, 118943, 118944, 118945 were selected from the ORFs having the nucleotide sequence represented by SEQ ID NO: 78579.

The probes having the nucleotide sequence represented by SEQ ID NOS: 118946, 118947, 118948, 118949, 118950, 118951, 118952, 118953, 118954, 118955, 118956 were selected from the ORFs having the nucleotide sequence represented by SEQ ID NO: 78580.

The probes having the nucleotide sequence represented by SEQ ID NOS: 118957, 118958, 118959, 118960, 118961, 118962, 118963, 118964, 118965, 118966, 118967 were selected from the ORFs having the nucleotide sequence represented by SEQ ID NO: 78581.

The probes having the nucleotide sequence represented by SEQ ID NOS: 118968, 118969, 118970, 118971, 118972, 118973, 118974, 118975, 118976, 118977, 118978 were selected from the ORFs having the nucleotide sequence represented by SEQ ID NO: 78582.

The probes having the nucleotide sequence represented by SEQ ID NOS: 118979, 118980, 118981, 118982, 118983, 118984, 118985, 118986, 118987, 118988, 118989 were selected from the ORFs having the nucleotide sequence represented by SEQ ID NO: 78583.

The probes having the nucleotide sequence represented by SEQ ID NOS: 118990, 118991, 118992, 118993, 118994, 118995, 118996, 118997, 118998, 118999, 119000 were selected from the ORFs having the nucleotide sequence represented by SEQ ID NO: 78584.

The probes having the nucleotide sequence represented by SEQ ID NOS: 119001, 119002, 119003, 119004, 119005, 119006, 119007, 119008, 119009, 119010, 119011 were selected from the ORFs having the nucleotide sequence represented by SEQ ID NO: 78585.

The probes having the nucleotide sequence represented by SEQ ID NOS: 119012, 119013, 119014, 119015, 119016, 119017, 119018, 119019, 119020, 119021, 119022 were selected from the ORFs having the nucleotide sequence represented by SEQ ID NO: 78586.

The probes having the nucleotide sequence represented by SEQ ID NOS: 119023, 119024, 119025, 119026, 119027, 119028, 119029, 119030, 119031, 119032, 119033 were selected from the ORFs having the nucleotide sequence represented by SEQ ID NO: 78587.

The probes having the nucleotide sequence represented by SEQ ID NOS: 119034, 119035, 119036, 119037, 119038, 119039, 119040, 119041, 119042, 119043, 119044 were selected from the ORFs having the nucleotide sequence represented by SEQ ID NO: 78588.

The probes having the nucleotide sequence represented by SEQ ID NOS: 119045, 119046, 119047, 119048, 119049, 119050, 119051, 119052, 119053, 119054, 119055, 119056, 119057, 119058, 119059, 119060, 119061, 119062, 119063, 119064, 119065, 119066 were selected from the ORFs having the nucleotide sequence represented by SEQ ID NO: 78589.

The probes having the nucleotide sequence represented by SEQ ID NOS: 119067, 119068, 119069, 119070, 119071, 119072, 119073, 119074, 119075, 119076, 119077 were selected from the ORFs having the nucleotide sequence represented by SEQ ID NO: 78590.

The probes having the nucleotide sequence represented by SEQ ID NOS: 119078, 119079, 119080, 119081, 119082, 119083, 119084, 119085, 119086, 119087, 119088 were selected from the ORFs having the nucleotide sequence represented by SEQ ID NO: 78591.

The probes having the nucleotide sequence represented by SEQ ID NOS: 119089, 119090, 119091, 119092, 119093, 119094, 119095, 119096, 119097, 119098, 119099 were selected from the ORFs having the nucleotide sequence represented by SEQ ID NO: 78592.

The probes having the nucleotide sequence represented by SEQ ID NOS: 119100, 119101, 119102, 119103, 119104, 119105, 119106, 119107, 119108, 119109, 119110 were selected from the ORFs having the nucleotide sequence represented by SEQ ID NO: 78593.

The probes having the nucleotide sequence represented by SEQ ID NOS: 119111, 119112, 119113, 119114, 119115, 119116, 119117, 119118, 119119, 119120, 119121 were selected from the ORFs having the nucleotide sequence represented by SEQ ID NO: 78594.

The probes having the nucleotide sequence represented by SEQ ID NOS: 119122, 119123, 119124, 119125, 119126, 119127, 119128, 119129, 119130, 119131, 119132 were selected from the ORFs having the nucleotide sequence represented by SEQ ID NO: 78595.

The probes having the nucleotide sequence represented by SEQ ID NOS: 119133, 119134, 119135, 119136, 119137, 119138, 119139, 119140, 119141, 119142, 119143 were selected from the ORFs having the nucleotide sequence represented by SEQ ID NO: 78596.

The probes having the nucleotide sequence represented by SEQ ID NOS: 119144, 119145, 119146, 119147, 119148, 119149, 119150, 119151, 119152, 119153, 119154 were selected from the ORFs having the nucleotide sequence represented by SEQ ID NO: 78597.

The probes having the nucleotide sequence represented by SEQ ID NOS: 119155, 119156, 119157, 119158, 119159, 119160, 119161, 119162, 119163, 119164, 119165 were selected from the ORFs having the nucleotide sequence represented by SEQ ID NO: 78598.

The probes having the nucleotide sequence represented by SEQ ID NOS: 119166, 119167, 119168, 119169, 119170, 119171, 119172, 119173, 119174, 119175, 119176 were selected from the ORFs having the nucleotide sequence represented by SEQ ID NO: 78599.

The probes having the nucleotide sequence represented by SEQ ID NOS: 119177, 119178, 119179, 119180, 119181, 119182, 119183, 119184, 119185, 119186, 119187 were selected from the ORFs having the nucleotide sequence represented by SEQ ID NO: 78600.

The probes having the nucleotide sequence represented by SEQ ID NOS: 119188, 119189, 119190, 119191, 119192, 119193, 119194, 119195, 119196, 119197, 119198 were selected from the ORFs having the nucleotide sequence represented by SEQ ID NO: 78601.

The probes having the nucleotide sequence represented by SEQ ID NOS: 119199, 119200, 119201, 119202, 119203, 119204, 119205, 119206, 119207, 119208, 119209 were selected from the ORFs having the nucleotide sequence represented by SEQ ID NO: 78602.

The probes having the nucleotide sequence represented by SEQ ID NOS: 119210, 119211, 119212, 119213, 119214, 119215, 119216, 119217, 119218, 119219, 119220 were selected from the ORFs having the nucleotide sequence represented by SEQ ID NO: 78603.

The probes having the nucleotide sequence represented by SEQ ID NOS: 119221, 119222, 119223, 119224, 119225, 119226, 119227, 119228, 119229, 119230, 119231 were selected from the ORFs having the nucleotide sequence represented by SEQ ID NO: 78604.

The probes having the nucleotide sequence represented by SEQ ID NOS: 119232, 119233, 119234, 119235, 119236, 119237, 119238, 119239, 119240, 119241, 119242 were selected from the ORFs having the nucleotide sequence represented by SEQ ID NO: 78605.

The probes having the nucleotide sequence represented by SEQ ID NOS: 119243, 119244, 119245, 119246, 119247, 119248, 119249, 119250, 119251, 119252, 119253 were selected from the ORFs having the nucleotide sequence represented by SEQ ID NO: 78606.

The probes having the nucleotide sequence represented by SEQ ID NOS: 119254, 119255, 119256, 119257, 119258, 119259, 119260, 119261, 119262, 119263, 119264 were selected from the ORFs having the nucleotide sequence represented by SEQ ID NO: 78607.

The probes having the nucleotide sequence represented by SEQ ID NOS: 119265, 119266, 119267, 119268, 119269, 119270, 119271, 119272, 119273, 119274, 119275 were selected from the ORFs having the nucleotide sequence represented by SEQ ID NO: 78608.

The probes having the nucleotide sequence represented by SEQ ID NOS: 119276, 119277, 119278, 119279, 119280, 119281, 119282, 119283, 119284, 119285, 119286 were selected from the ORFs having the nucleotide sequence represented by SEQ ID NO: 78609.

The probes having the nucleotide sequence represented by SEQ ID NOS: 119287, 119288, 119289, 119290, 119291, 119292, 119293, 119294, 119295, 119296, 119297 were selected from the ORFs having the nucleotide sequence represented by SEQ ID NO: 78610.

The probes having the nucleotide sequence represented by SEQ ID NOS: 119298, 119299, 119300, 119301, 119302, 119303, 119304, 119305, 119306, 119307, 119308 were selected from the ORFs having the nucleotide sequence represented by SEQ ID NO: 78611.

The probes having the nucleotide sequence represented by SEQ ID NOS: 119309, 119310, 119311, 119312, 119313, 119314, 119315, 119316, 119317, 119318, 119319 were selected from the ORFs having the nucleotide sequence represented by SEQ ID NO: 78612.

The probes having the nucleotide sequence represented by SEQ ID NOS: 119320, 119321, 119322, 119323, 119324, 119325, 119326, 119327, 119328, 119329, 119330 were selected from the ORFs having the nucleotide sequence represented by SEQ ID NO: 78613.

The probes having the nucleotide sequence represented by SEQ ID NOS: 119331, 119332, 119333, 119334, 119335, 119336, 119337, 119338, 119339, 119340, 119341 were selected from the ORFs having the nucleotide sequence represented by SEQ ID NO: 78614.

The probes having the nucleotide sequence represented by SEQ ID NOS: 119342, 119343, 119344, 119345, 119346, 119347, 119348, 119349, 119350, 119351, 119352 were selected from the ORFs having the nucleotide sequence represented by SEQ ID NO: 78615.

The probes having the nucleotide sequence represented by SEQ ID NOS: 119353, 119354, 119355, 119356, 119357, 119358, 119359, 119360, 119361, 119362, 119363 were selected from the ORFs having the nucleotide sequence represented by SEQ ID NO: 78616.

The probes having the nucleotide sequence represented by SEQ ID NOS: 119364, 119365, 119366, 119367, 119368, 119369, 119370, 119371, 119372, 119373, 119374 were selected from the ORFs having the nucleotide sequence represented by SEQ ID NO: 78617.

The probes having the nucleotide sequence represented by SEQ ID NOS: 119375, 119376, 119377, 119378, 119379, 119380, 119381, 119382, 119383, 119384, 119385 were selected from the ORFs having the nucleotide sequence represented by SEQ ID NO: 78618.

The probes having the nucleotide sequence represented by SEQ ID NOS: 119386, 119387, 119388, 119389, 119390, 119391, 119392, 119393, 119394, 119395, 119396 were selected from the ORFs having the nucleotide sequence represented by SEQ ID NO: 78619.

The probes having the nucleotide sequence represented by SEQ ID NOS: 119397, 119398, 119399, 119400, 119401, 119402, 119403, 119404, 119405, 119406, 119407 were selected from the ORFs having the nucleotide sequence represented by SEQ ID NO: 78620.

The probes having the nucleotide sequence represented by SEQ ID NOS: 119408, 119409, 119410, 119411, 119412, 119413, 119414, 119415, 119416, 119417, 119418 were selected from the ORFs having the nucleotide sequence represented by SEQ ID NO: 78621.

The probes having the nucleotide sequence represented by SEQ ID NOS: 119419, 119420, 119421, 119422, 119423, 119424, 119425, 119426, 119427, 119428, 119429 were selected from the ORFs having the nucleotide sequence represented by SEQ ID NO: 78622.

The probes having the nucleotide sequence represented by SEQ ID NOS: 119430, 119431, 119432, 119433, 119434, 119435, 119436, 119437, 119438, 119439, 119440 were selected from the ORFs having the nucleotide sequence represented by SEQ ID NO: 78623.

The probes having the nucleotide sequence represented by SEQ ID NOS: 119441, 119442, 119443, 119444, 119445, 119446, 119447, 119448, 119449, 119450, 119451 were selected from the ORFs having the nucleotide sequence represented by SEQ ID NO: 78624.

The probes having the nucleotide sequence represented by SEQ ID NOS: 119452, 119453, 119454, 119455, 119456, 119457, 119458, 119459, 119460, 119461, 119462 were selected from the ORFs having the nucleotide sequence represented by SEQ ID NO: 78625.

The probes having the nucleotide sequence represented by SEQ ID NOS: 119463, 119464, 119465, 119466, 119467, 119468, 119469, 119470, 119471, 119472, 119473 were selected from the ORFs having the nucleotide sequence represented by SEQ ID NO: 78626.

The probes having the nucleotide sequence represented by SEQ ID NOS: 119474, 119475, 119476, 119477, 119478, 119479, 119480, 119481, 119482, 119483, 119484 were selected from the ORFs having the nucleotide sequence represented by SEQ ID NO: 78627.

The probes having the nucleotide sequence represented by SEQ ID NOS: 119485, 119486, 119487, 119488, 119489, 119490, 119491, 119492, 119493, 119494, 119495 were selected from the ORFs having the nucleotide sequence represented by SEQ ID NO: 78628.

The probes having the nucleotide sequence represented by SEQ ID NOS: 119496, 119497, 119498, 119499, 119500, 119501, 119502, 119503, 119504, 119505, 119506 were selected from the ORFs having the nucleotide sequence represented by SEQ ID NO: 78629.

The probes having the nucleotide sequence represented by SEQ ID NOS: 119507, 119508, 119509, 119510, 119511, 119512, 119513, 119514, 119515, 119516, 119517 were selected from the ORFs having the nucleotide sequence represented by SEQ ID NO: 78630.

The probes having the nucleotide sequence represented by SEQ ID NOS: 119518, 119519, 119520, 119521, 119522, 119523, 119524, 119525, 119526, 119527, 119528 were selected from the ORFs having the nucleotide sequence represented by SEQ ID NO: 78631.

The probes having the nucleotide sequence represented by SEQ ID NOS: 119529, 119530, 119531, 119532, 119533, 119534, 119535, 119536, 119537, 119538, 119539 were selected from the ORFs having the nucleotide sequence represented by SEQ ID NO: 78632.

The probes having the nucleotide sequence represented by SEQ ID NOS: 119540, 119541, 119542, 119543, 119544, 119545, 119546, 119547, 119548, 119549, 119550 were selected from the ORFs having the nucleotide sequence represented by SEQ ID NO: 78633.

The probes having the nucleotide sequence represented by SEQ ID NOS: 119551, 119552, 119553, 119554, 119555, 119556, 119557, 119558, 119559, 119560, 119561 were selected from the ORFs having the nucleotide sequence represented by SEQ ID NO: 78634.

The probes having the nucleotide sequence represented by SEQ ID NOS: 119562, 119563, 119564, 119565, 119566, 119567, 119568, 119569, 119570, 119571, 119572 were selected from the ORFs having the nucleotide sequence represented by SEQ ID NO: 78635.

The probes having the nucleotide sequence represented by SEQ ID NOS: 119573, 119574, 119575, 119576, 119577, 119578, 119579, 119580, 119581, 119582, 119583 were selected from the ORFs having the nucleotide sequence represented by SEQ ID NO: 78636.

The probes having the nucleotide sequence represented by SEQ ID NOS: 119584, 119585, 119586, 119587, 119588, 119589, 119590, 119591, 119592, 119593, 119594 were selected from the ORFs having the nucleotide sequence represented by SEQ ID NO: 78637.

The probes having the nucleotide sequence represented by SEQ ID NOS: 119595, 119596, 119597, 119598, 119599, 119600, 119601, 119602, 119603, 119604, 119605 were selected from the ORFs having the nucleotide sequence represented by SEQ ID NO: 78638.

The probes having the nucleotide sequence represented by SEQ ID NOS: 119606, 119607, 119608, 119609, 119610, 119611, 119612, 119613, 119614, 119615, 119616 were selected from the ORFs having the nucleotide sequence represented by SEQ ID NO: 78639.

The probes having the nucleotide sequence represented by SEQ ID NOS: 119617, 119618, 119619, 119620, 119621, 119622, 119623, 119624, 119625, 119626, 119627 were selected from the ORFs having the nucleotide sequence represented by SEQ ID NO: 78640.

The probes having the nucleotide sequence represented by SEQ ID NOS: 119628, 119629, 119630, 119631, 119632, 119633, 119634, 119635, 119636, 119637, 119638 were selected from the ORFs having the nucleotide sequence represented by SEQ ID NO: 78641.

The probes having the nucleotide sequence represented by SEQ ID NOS: 119639, 119640, 119641, 119642, 119643, 119644, 119645, 119646, 119647, 119648, 119649 were selected from the ORFs having the nucleotide sequence represented by SEQ ID NO: 78642.

The probes having the nucleotide sequence represented by SEQ ID NOS: 119650, 119651, 119652, 119653, 119654, 119655, 119656, 119657, 119658, 119659, 119660 were selected from the ORFs having the nucleotide sequence represented by SEQ ID NO: 78643.

The probes having the nucleotide sequence represented by SEQ ID NOS: 119661, 119662, 119663, 119664, 119665, 119666, 119667, 119668, 119669, 119670, 119671 were selected from the ORFs having the nucleotide sequence represented by SEQ ID NO: 78644.

The probes having the nucleotide sequence represented by SEQ ID NOS: 119672, 119673, 119674, 119675, 119676, 119677, 119678, 119679, 119680, 119681, 119682 were selected from the ORFs having the nucleotide sequence represented by SEQ ID NO: 78645.

The probes having the nucleotide sequence represented by SEQ ID NOS: 119683, 119684, 119685, 119686, 119687, 119688, 119689, 119690, 119691, 119692, 119693 were selected from the ORFs having the nucleotide sequence represented by SEQ ID NO: 78646.

The probes having the nucleotide sequence represented by SEQ ID NOS: 119694, 119695, 119696, 119697, 119698, 119699, 119700, 119701, 119702, 119703, 119704 were selected from the ORFs having the nucleotide sequence represented by SEQ ID NO: 78647.

The probes having the nucleotide sequence represented by SEQ ID NOS: 119705, 119706, 119707, 119708, 119709, 119710, 119711, 119712, 119713, 119714, 119715 were selected from the ORFs having the nucleotide sequence represented by SEQ ID NO: 78648.

The probes having the nucleotide sequence represented by SEQ ID NOS: 119716, 119717, 119718, 119719, 119720, 119721, 119722, 119723, 119724, 119725, 119726 were selected from the ORFs having the nucleotide sequence represented by SEQ ID NO: 78649.

The probes having the nucleotide sequence represented by SEQ ID NOS: 119727, 119728, 119729, 119730, 119731, 119732, 119733, 119734, 119735, 119736, 119737 were selected from the ORFs having the nucleotide sequence represented by SEQ ID NO: 78650.

The probes having the nucleotide sequence represented by SEQ ID NOS: 119738, 119739, 119740, 119741, 119742, 119743, 119744, 119745, 119746, 119747, 119748 were selected from the ORFs having the nucleotide sequence represented by SEQ ID NO: 78651.

The probes having the nucleotide sequence represented by SEQ ID NOS: 119749, 119750, 119751, 119752, 119753, 119754, 119755, 119756, 119757, 119758, 119759 were selected from the ORFs having the nucleotide sequence represented by SEQ ID NO: 78652.

The probes having the nucleotide sequence represented by SEQ ID NOS: 119760, 119761, 119762, 119763, 119764, 119765, 119766, 119767, 119768 were selected from the ORFs having the nucleotide sequence represented by SEQ ID NO: 78653.

The probes having the nucleotide sequence represented by SEQ ID NOS: 119769, 119770, 119771, 119772, 119773, 119774, 119775, 119776, 119777, 119778, 119779 were selected from the ORFs having the nucleotide sequence represented by SEQ ID NO: 78654.

The probes having the nucleotide sequence represented by SEQ ID NOS: 119780, 119781, 119782, 119783, 119784, 119785, 119786, 119787, 119788, 119789, 119790 were selected from the ORFs having the nucleotide sequence represented by SEQ ID NO: 78655.

The probes having the nucleotide sequence represented by SEQ ID NOS: 119791, 119792, 119793, 119794, 119795, 119796, 119797, 119798, 119799, 119800, 119801 were selected from the ORFs having the nucleotide sequence represented by SEQ ID NO: 78656.

The probes having the nucleotide sequence represented by SEQ ID NOS: 119802, 119803, 119804, 119805, 119806, 119807, 119808, 119809, 119810, 119811, 119812 were selected from the ORFs having the nucleotide sequence represented by SEQ ID NO: 78657.

The probes having the nucleotide sequence represented by SEQ ID NOS: 119813, 119814, 119815, 119816, 119817, 119818, 119819, 119820, 119821, 119822, 119823 were selected from the ORFs having the nucleotide sequence represented by SEQ ID NO: 78658.

The probes having the nucleotide sequence represented by SEQ ID NOS: 119824, 119825, 119826, 119827, 119828, 119829, 119830, 119831, 119832, 119833, 119834 were selected from the ORFs having the nucleotide sequence represented by SEQ ID NO: 78659.

The probes having the nucleotide sequence represented by SEQ ID NOS: 119835, 119836, 119837, 119838, 119839, 119840, 119841, 119842, 119843, 119844, 119845 were selected from the ORFs having the nucleotide sequence represented by SEQ ID NO: 78660.

The probes having the nucleotide sequence represented by SEQ ID NOS: 119846, 119847, 119848, 119849, 119850, 119851, 119852, 119853, 119854, 119855, 119856 were selected from the ORFs having the nucleotide sequence represented by SEQ ID NO: 78661.

The probes having the nucleotide sequence represented by SEQ ID NOS: 119857, 119858, 119859, 119860, 119861, 119862, 119863, 119864, 119865, 119866, 119867 were selected from the ORFs having the nucleotide sequence represented by SEQ ID NO: 78662.

The probes having the nucleotide sequence represented by SEQ ID NOS: 119868, 119869, 119870, 119871, 119872, 119873, 119874, 119875, 119876, 119877, 119878 were selected from the ORFs having the nucleotide sequence represented by SEQ ID NO: 78663.

The probes having the nucleotide sequence represented by SEQ ID NOS: 119879, 119880, 119881, 119882, 119883, 119884, 119885, 119886, 119887, 119888, 119889 were selected from the ORFs having the nucleotide sequence represented by SEQ ID NO: 78664.

The probes having the nucleotide sequence represented by SEQ ID NOS: 119890, 119891, 119892, 119893, 119894, 119895, 119896, 119897, 119898, 119899, 119900 were selected from the ORFs having the nucleotide sequence represented by SEQ ID NO: 78665.

The probes having the nucleotide sequence represented by SEQ ID NOS: 119901, 119902, 119903, 119904, 119905, 119906, 119907, 119908, 119909, 119910, 119911 were selected from the ORFs having the nucleotide sequence represented by SEQ ID NO: 78666.

The probes having the nucleotide sequence represented by SEQ ID NOS: 119912, 119913, 119914, 119915, 119916, 119917, 119918, 119919, 119920, 119921, 119922 were selected from the ORFs having the nucleotide sequence represented by SEQ ID NO: 78667.

The probes having the nucleotide sequence represented by SEQ ID NOS: 119923, 119924, 119925, 119926, 119927, 119928, 119929, 119930, 119931, 119932, 119933 were selected from the ORFs having the nucleotide sequence represented by SEQ ID NO: 78668.

The probes having the nucleotide sequence represented by SEQ ID NOS: 119934, 119935, 119936, 119937, 119938, 119939, 119940, 119941, 119942, 119943, 119944 were selected from the ORFs having the nucleotide sequence represented by SEQ ID NO: 78669.

The probes having the nucleotide sequence represented by SEQ ID NOS: 119945, 119946, 119947, 119948, 119949, 119950, 119951, 119952, 119953, 119954, 119955 were selected from the ORFs having the nucleotide sequence represented by SEQ ID NO: 78670.

The probes having the nucleotide sequence represented by SEQ ID NOS: 119956, 119957, 119958, 119959, 119960, 119961, 119962, 119963, 119964, 119965, 119966 were selected from the ORFs having the nucleotide sequence represented by SEQ ID NO: 78671.

The probes having the nucleotide sequence represented by SEQ ID NOS: 119967, 119968, 119969, 119970, 119971, 119972, 119973, 119974, 119975, 119976, 119977 were selected from the ORFs having the nucleotide sequence represented by SEQ ID NO: 78672.

The probes having the nucleotide sequence represented by SEQ ID NOS: 119978, 119979, 119980, 119981, 119982, 119983, 119984, 119985, 119986, 119987, 119988 were selected from the ORFs having the nucleotide sequence represented by SEQ ID NO: 78673.

The probes having the nucleotide sequence represented by SEQ ID NOS: 119989, 119990, 119991, 119992, 119993, 119994, 119995, 119996, 119997, 119998, 119999 were selected from the ORFs having the nucleotide sequence represented by SEQ ID NO: 78674.

The probes having the nucleotide sequence represented by SEQ ID NOS: 120000, 120001, 120002, 120003, 120004, 120005, 120006, 120007, 120008, 120009, 120010 were selected from the ORFs having the nucleotide sequence represented by SEQ ID NO: 78675.

The probes having the nucleotide sequence represented by SEQ ID NOS: 120011, 120012, 120013, 120014, 120015, 120016, 120017, 120018, 120019, 120020, 120021 were selected from the ORFs having the nucleotide sequence represented by SEQ ID NO: 78676.

The probes having the nucleotide sequence represented by SEQ ID NOS: 120022, 120023, 120024, 120025, 120026, 120027, 120028, 120029, 120030, 120031, 120032 were selected from the ORFs having the nucleotide sequence represented by SEQ ID NO: 78677.

The probes having the nucleotide sequence represented by SEQ ID NOS: 120033, 120034, 120035, 120036, 120037, 120038, 120039, 120040, 120041, 120042, 120043 were selected from the ORFs having the nucleotide sequence represented by SEQ ID NO: 78678.

The probes having the nucleotide sequence represented by SEQ ID NOS: 120044, 120045, 120046, 120047, 120048, 120049, 120050, 120051, 120052, 120053, 120054 were selected from the ORFs having the nucleotide sequence represented by SEQ ID NO: 78679.

The probes having the nucleotide sequence represented by SEQ ID NOS: 120055, 120056, 120057, 120058, 120059, 120060, 120061, 120062, 120063, 120064, 120065 were selected from the ORFs having the nucleotide sequence represented by SEQ ID NO: 78680.

The probes having the nucleotide sequence represented by SEQ ID NOS: 120066, 120067, 120068, 120069, 120070, 120071, 120072, 120073, 120074, 120075, 120076 were selected from the ORFs having the nucleotide sequence represented by SEQ ID NO: 78681.

The probes having the nucleotide sequence represented by SEQ ID NOS: 120077, 120078, 120079, 120080, 120081, 120082, 120083, 120084, 120085, 120086, 120087 were selected from the ORFs having the nucleotide sequence represented by SEQ ID NO: 78682.

The probes having the nucleotide sequence represented by SEQ ID NOS: 120088, 120089, 120090, 120091, 120092, 120093, 120094, 120095, 120096, 120097, 120098 were selected from the ORFs having the nucleotide sequence represented by SEQ ID NO: 78683.

The probes having the nucleotide sequence represented by SEQ ID NOS: 120099, 120100, 120101, 120102, 120103, 120104, 120105, 120106, 120107, 120108, 120109 were selected from the ORFs having the nucleotide sequence represented by SEQ ID NO: 78684.

The probes having the nucleotide sequence represented by SEQ ID NOS: 120110, 120111, 120112, 120113, 120114, 120115, 120116, 120117, 120118, 120119, 120120 were selected from the ORFs having the nucleotide sequence represented by SEQ ID NO: 78685.

The probes having the nucleotide sequence represented by SEQ ID NOS: 120121, 120122, 120123, 120124, 120125, 120126, 120127, 120128, 120129, 120130, 120131 were selected from the ORFs having the nucleotide sequence represented by SEQ ID NO: 78686.

The probes having the nucleotide sequence represented by SEQ ID NOS: 120132, 120133, 120134, 120135, 120136, 120137, 120138, 120139, 120140, 120141, 120142 were selected from the ORFs having the nucleotide sequence represented by SEQ ID NO: 78687.

The probes having the nucleotide sequence represented by SEQ ID NOS: 120143, 120144, 120145, 120146, 120147, 120148, 120149, 120150, 120151, 120152, 120153 were selected from the ORFs having the nucleotide sequence represented by SEQ ID NO: 78688.

The probes having the nucleotide sequence represented by SEQ ID NOS: 120154, 120155, 120156, 120157, 120158, 120159, 120160, 120161, 120162, 120163, 120164 were selected from the ORFs having the nucleotide sequence represented by SEQ ID NO: 78689.

The probes having the nucleotide sequence represented by SEQ ID NOS: 120165, 120166, 120167, 120168, 120169, 120170, 120171, 120172, 120173, 120174, 120175 were selected from the ORFs having the nucleotide sequence represented by SEQ ID NO: 78690.

The probes having the nucleotide sequence represented by SEQ ID NOS: 120176, 120177, 120178, 120179, 120180, 120181, 120182, 120183, 120184, 120185, 120186 were selected from the ORFs having the nucleotide sequence represented by SEQ ID NO: 78691.

The probes having the nucleotide sequence represented by SEQ ID NOS: 120187, 120188, 120189, 120190, 120191, 120192, 120193, 120194, 120195, 120196, 120197 were selected from the ORFs having the nucleotide sequence represented by SEQ ID NO: 78692.

The probes having the nucleotide sequence represented by SEQ ID NOS: 120198, 120199, 120200, 120201, 120202, 120203, 120204, 120205, 120206, 120207, 120208 were selected from the ORFs having the nucleotide sequence represented by SEQ ID NO: 78693.

The probes having the nucleotide sequence represented by SEQ ID NOS: 120209, 120210, 120211, 120212, 120213, 120214, 120215, 120216, 120217, 120218, 120219 were selected from the ORFs having the nucleotide sequence represented by SEQ ID NO: 78694.

The probes having the nucleotide sequence represented by SEQ ID NOS: 120220, 120221, 120222, 120223, 120224, 120225, 120226, 120227, 120228, 120229, 120230 were selected from the ORFs having the nucleotide sequence represented by SEQ ID NO: 78695.

The probes having the nucleotide sequence represented by SEQ ID NOS: 120231, 120232, 120233, 120234, 120235, 120236, 120237, 120238, 120239, 120240, 120241 were selected from the ORFs having the nucleotide sequence represented by SEQ ID NO: 78696.

The probes having the nucleotide sequence represented by SEQ ID NOS: 120242, 120243, 120244, 120245, 120246, 120247, 120248, 120249, 120250, 120251, 120252 were selected from the ORFs having the nucleotide sequence represented by SEQ ID NO: 78697.

The probes having the nucleotide sequence represented by SEQ ID NOS: 120253, 120254, 120255, 120256, 120257, 120258, 120259, 120260, 120261, 120262, 120263 were selected from the ORFs having the nucleotide sequence represented by SEQ ID NO: 78698.

The probes having the nucleotide sequence represented by SEQ ID NOS: 120264, 120265, 120266, 120267, 120268, 120269, 120270, 120271, 120272, 120273, 120274 were selected from the ORFs having the nucleotide sequence represented by SEQ ID NO: 78699.

The probes having the nucleotide sequence represented by SEQ ID NOS: 120275, 120276, 120277, 120278, 120279, 120280, 120281, 120282, 120283, 120284, 120285 were selected from the ORFs having the nucleotide sequence represented by SEQ ID NO: 78700.

The probes having the nucleotide sequence represented by SEQ ID NOS: 120286, 120287, 120288, 120289, 120290, 120291, 120292, 120293, 120294, 120295, 120296 were selected from the ORFs having the nucleotide sequence represented by SEQ ID NO: 78701.

The probes having the nucleotide sequence represented by SEQ ID NOS: 120297, 120298, 120299, 120300, 120301, 120302, 120303, 120304, 120305, 120306, 120307 were selected from the ORFs having the nucleotide sequence represented by SEQ ID NO: 78702.

The probes having the nucleotide sequence represented by SEQ ID NOS: 120308, 120309, 120310, 120311, 120312, 120313, 120314, 120315, 120316, 120317, 120318 were selected from the ORFs having the nucleotide sequence represented by SEQ ID NO: 78703.

The probes having the nucleotide sequence represented by SEQ ID NOS: 120319, 120320, 120321, 120322, 120323, 120324, 120325, 120326, 120327, 120328, 120329 were selected from the ORFs having the nucleotide sequence represented by SEQ ID NO: 78704.

The probes having the nucleotide sequence represented by SEQ ID NOS: 120330, 120331, 120332, 120333, 120334, 120335, 120336, 120337, 120338, 120339, 120340 were selected from the ORFs having the nucleotide sequence represented by SEQ ID NO: 78705.

The probes having the nucleotide sequence represented by SEQ ID NOS: 120341, 120342, 120343, 120344, 120345, 120346, 120347, 120348, 120349, 120350, 120351 were selected from the ORFs having the nucleotide sequence represented by SEQ ID NO: 78706.

The probes having the nucleotide sequence represented by SEQ ID NOS: 120352, 120353, 120354, 120355, 120356, 120357, 120358, 120359, 120360, 120361, 120362 were selected from the ORFs having the nucleotide sequence represented by SEQ ID NO: 78707.

The probes having the nucleotide sequence represented by SEQ ID NOS: 120363, 120364, 120365, 120366, 120367, 120368, 120369, 120370, 120371, 120372, 120373 were selected from the ORFs having the nucleotide sequence represented by SEQ ID NO: 78708.

The probes having the nucleotide sequence represented by SEQ ID NOS: 120374, 120375, 120376, 120377, 120378, 120379, 120380, 120381, 120382, 120383, 120384 were selected from the ORFs having the nucleotide sequence represented by SEQ ID NO: 78709.

The probes having the nucleotide sequence represented by SEQ ID NOS: 120385, 120386, 120387, 120388, 120389, 120390, 120391, 120392, 120393, 120394, 120395 were selected from the ORFs having the nucleotide sequence represented by SEQ ID NO: 78710.

The probes having the nucleotide sequence represented by SEQ ID NOS: 120396, 120397, 120398, 120399, 120400, 120401, 120402, 120403, 120404, 120405, 120406 were selected from the ORFs having the nucleotide sequence represented by SEQ ID NO: 78711.

The probes having the nucleotide sequence represented by SEQ ID NOS: 120407, 120408, 120409, 120410, 120411, 120412, 120413, 120414, 120415, 120416, 120417 were selected from the ORFs having the nucleotide sequence represented by SEQ ID NO: 78712.

The probes having the nucleotide sequence represented by SEQ ID NOS: 120418, 120419, 120420, 120421, 120422, 120423, 120424, 120425, 120426, 120427, 120428 were selected from the ORFs having the nucleotide sequence represented by SEQ ID NO: 78713.

The probes having the nucleotide sequence represented by SEQ ID NOS: 120429, 120430, 120431, 120432, 120433, 120434, 120435, 120436, 120437, 120438, 120439 were selected from the ORFs having the nucleotide sequence represented by SEQ ID NO: 78714.

The probes having the nucleotide sequence represented by SEQ ID NOS: 120440, 120441, 120442, 120443, 120444, 120445, 120446, 120447, 120448, 120449, 120450 were selected from the ORFs having the nucleotide sequence represented by SEQ ID NO: 78715.

The probes having the nucleotide sequence represented by SEQ ID NOS: 120451, 120452, 120453, 120454, 120455, 120456, 120457, 120458, 120459, 120460, 120461 were selected from the ORFs having the nucleotide sequence represented by SEQ ID NO: 78716.

The probes having the nucleotide sequence represented by SEQ ID NOS: 120462, 120463, 120464, 120465, 120466, 120467, 120468, 120469, 120470, 120471, 120472 were selected from the ORFs having the nucleotide sequence represented by SEQ ID NO: 78717.

The probes having the nucleotide sequence represented by SEQ ID NOS: 120473, 120474, 120475, 120476, 120477, 120478, 120479, 120480, 120481, 120482, 120483 were selected from the ORFs having the nucleotide sequence represented by SEQ ID NO: 78718.

The probes having the nucleotide sequence represented by SEQ ID NOS: 120484, 120485, 120486, 120487, 120488, 120489, 120490, 120491, 120492, 120493, 120494 were selected from the ORFs having the nucleotide sequence represented by SEQ ID NO: 78719.

The probes having the nucleotide sequence represented by SEQ ID NOS: 120495, 120496, 120497, 120498, 120499, 120500, 120501, 120502, 120503, 120504, 120505 were selected from the ORFs having the nucleotide sequence represented by SEQ ID NO: 78720.

The probes having the nucleotide sequence represented by SEQ ID NOS: 120506, 120507, 120508, 120509, 120510, 120511, 120512, 120513, 120514, 120515, 120516 were selected from the ORFs having the nucleotide sequence represented by SEQ ID NO: 78721.

The probes having the nucleotide sequence represented by SEQ ID NOS: 120517, 120518, 120519, 120520, 120521, 120522, 120523, 120524, 120525, 120526, 120527 were selected from the ORFs having the nucleotide sequence represented by SEQ ID NO: 78722.

The probes having the nucleotide sequence represented by SEQ ID NOS: 120528, 120529, 120530, 120531, 120532, 120533, 120534, 120535, 120536, 120537, 120538 were selected from the ORFs having the nucleotide sequence represented by SEQ ID NO: 78723.

The probes having the nucleotide sequence represented by SEQ ID NOS: 120539, 120540, 120541, 120542, 120543, 120544, 120545, 120546, 120547, 120548, 120549 were selected from the ORFs having the nucleotide sequence represented by SEQ ID NO: 78724.

The probes having the nucleotide sequence represented by SEQ ID NOS: 120550, 120551, 120552, 120553, 120554, 120555, 120556, 120557, 120558, 120559, 120560 were selected from the ORFs having the nucleotide sequence represented by SEQ ID NO: 78725.

The probes having the nucleotide sequence represented by SEQ ID NOS: 120561, 120562, 120563, 120564, 120565, 120566, 120567, 120568, 120569, 120570, 120571 were selected from the ORFs having the nucleotide sequence represented by SEQ ID NO: 78726.

The probes having the nucleotide sequence represented by SEQ ID NOS: 120572, 120573, 120574, 120575, 120576, 120577, 120578, 120579, 120580, 120581, 120582 were selected from the ORFs having the nucleotide sequence represented by SEQ ID NO: 78727.

The probes having the nucleotide sequence represented by SEQ ID NOS: 120583, 120584, 120585, 120586, 120587, 120588, 120589, 120590, 120591, 120592, 120593 were selected from the ORFs having the nucleotide sequence represented by SEQ ID NO: 78728.

The probes having the nucleotide sequence represented by SEQ ID NOS: 120594, 120595, 120596, 120597, 120598, 120599, 120600, 120601, 120602, 120603, 120604 were selected from the ORFs having the nucleotide sequence represented by SEQ ID NO: 78729.

The probes having the nucleotide sequence represented by SEQ ID NOS: 120605, 120606, 120607, 120608, 120609, 120610, 120611, 120612, 120613, 120614, 120615 were selected from the ORFs having the nucleotide sequence represented by SEQ ID NO: 78730.

The probes having the nucleotide sequence represented by SEQ ID NOS: 120616, 120617, 120618, 120619, 120620, 120621, 120622, 120623, 120624, 120625, 120626 were selected from the ORFs having the nucleotide sequence represented by SEQ. ID NO: 78731.

The probes having the nucleotide sequence represented by SEQ ID NOS: 120627, 120628, 120629, 120630, 120631, 120632, 120633, 120634, 120635, 120636, 120637 were selected from the ORFs having the nucleotide sequence represented by SEQ ID NO: 78732.

The probes having the nucleotide sequence represented by SEQ ID NOS: 120638, 120639, 120640, 120641, 120642, 120643, 120644, 120645, 120646, 120647, 120648 were selected from the ORFs having the nucleotide sequence represented by SEQ ID NO: 78733.

The probes having the nucleotide sequence represented by SEQ ID NOS: 120649, 120650, 120651, 120652, 120653, 120654, 120655, 120656, 120657, 120658, 120659 were selected from the ORFs having the nucleotide sequence represented by SEQ ID NO: 78734.

The probes having the nucleotide sequence represented by SEQ ID NOS: 120660, 120661, 120662, 120663, 120664, 120665, 120666, 120667, 120668, 120669, 120670 were selected from the ORFs having the nucleotide sequence represented by SEQ ID NO: 78735.

The probes having the nucleotide sequence represented by SEQ ID NOS: 120671, 120672, 120673, 120674, 120675, 120676, 120677, 120678, 120679, 120680, 120681 were selected from the ORFs having the nucleotide sequence represented by SEQ ID NO: 78736.

The probes having the nucleotide sequence represented by SEQ ID NOS: 120682, 120683, 120684, 120685, 120686, 120687, 120688, 120689, 120690, 120691, 120692 were selected from the ORFs having the nucleotide sequence represented by SEQ ID NO: 78737.

The probes having the nucleotide sequence represented by SEQ ID NOS: 120693, 120694, 120695, 120696, 120697, 120698, 120699, 120700, 120701, 120702, 120703 were selected from the ORFs having the nucleotide sequence represented by SEQ ID NO: 78738.

The probes having the nucleotide sequence represented by SEQ ID NOS: 120704, 120705, 120706, 120707, 120708, 120709, 120710, 120711, 120712, 120713, 120714 were selected from the ORFs having the nucleotide sequence represented by SEQ ID NO: 78739.

The probes having the nucleotide sequence represented by SEQ ID NOS: 120715, 120716, 120717, 120718, 120719, 120720, 120721, 120722, 120723, 120724, 120725 were selected from the ORFs having the nucleotide sequence represented by SEQ ID NO: 78740.

The probes having the nucleotide sequence represented by SEQ ID NOS: 120726, 120727, 120728, 120729, 120730, 120731, 120732, 120733, 120734, 120735, 120736 were selected from the ORFs having the nucleotide sequence represented by SEQ ID NO: 78741.

The probes having the nucleotide sequence represented by SEQ ID NOS: 120737, 120738, 120739, 120740, 120741, 120742, 120743, 120744, 120745, 120746, 120747 were selected from the ORFs having the nucleotide sequence represented by SEQ ID NO: 78742.

The probes having the nucleotide sequence represented by SEQ ID NOS: 120748, 120749, 120750, 120751, 120752, 120753, 120754, 120755, 120756, 120757, 120758 were selected from the ORFs having the nucleotide sequence represented by SEQ ID NO: 78743.

The probes having the nucleotide sequence represented by SEQ ID NOS: 120759, 120760, 120761, 120762, 120763, 120764, 120765, 120766, 120767, 120768, 120769 were selected from the ORFs having the nucleotide sequence represented by SEQ ID NO: 78744.

The probes having the nucleotide sequence represented by SEQ ID NOS: 120770, 120771, 120772, 120773, 120774, 120775, 120776, 120777, 120778, 120779, 120780 were selected from the ORFs having the nucleotide sequence represented by SEQ ID NO: 78745.

The probes having the nucleotide sequence represented by SEQ ID NOS: 120781, 120782, 120783, 120784, 120785, 120786, 120787, 120788, 120789, 120790, 120791 were selected from the ORFs having the nucleotide sequence represented by SEQ ID NO: 78746.

The probes having the nucleotide sequence represented by SEQ ID NOS: 120792, 120793, 120794, 120795, 120796, 120797, 120798, 120799, 120800, 120801, 120802 were selected from the ORFs having the nucleotide sequence represented by SEQ ID NO: 78747.

The probes having the nucleotide sequence represented by SEQ ID NOS: 120803, 120804, 120805, 120806, 120807, 120808, 120809, 120810, 120811, 120812, 120813 were selected from the ORFs having the nucleotide sequence represented by SEQ ID NO: 78748.

The probes having the nucleotide sequence represented by SEQ ID NOS: 120814, 120815, 120816, 120817, 120818, 120819, 120820, 120821, 120822, 120823, 120824 were selected from the ORFs having the nucleotide sequence represented by SEQ ID NO: 78749.

The probes having the nucleotide sequence represented by SEQ ID NOS: 120825, 120826, 120827, 120828, 120829, 120830, 120831, 120832, 120833, 120834, 120835 were selected from the ORFs having the nucleotide sequence represented by SEQ ID NO: 78750.

The probes having the nucleotide sequence represented by SEQ ID NOS: 120836, 120837, 120838, 120839, 120840, 120841, 120842, 120843, 120844, 120845, 120846 were selected from the ORFs having the nucleotide sequence represented by SEQ ID NO: 78751.

The probes having the nucleotide sequence represented by SEQ ID NOS: 120847, 120848, 120849, 120850, 120851, 120852, 120853, 120854, 120855, 120856, 120857 were selected from the ORFs having the nucleotide sequence represented by SEQ ID NO: 78752.

The probes having the nucleotide sequence represented by SEQ ID NOS: 120858, 120859, 120860, 120861, 120862, 120863, 120864, 120865, 120866, 120867, 120868 were selected from the ORFs having the nucleotide sequence represented by SEQ ID NO: 78753.

The probes having the nucleotide sequence represented by SEQ ID NOS: 120869, 120870, 120871, 120872, 120873, 120874, 120875, 120876, 120877, 120878, 120879 were selected from the ORFs having the nucleotide sequence represented by SEQ ID NO: 78754.

The probes having the nucleotide sequence represented by SEQ ID NOS: 120880, 120881, 120882, 120883, 120884, 120885, 120886, 120887, 120888, 120889, 120890 were selected from the ORFs having the nucleotide sequence represented by SEQ ID NO: 78755.

The probes having the nucleotide sequence represented by SEQ ID NOS: 120891, 120892, 120893, 120894, 120895, 120896, 120897, 120898, 120899, 120900, 120901, 120902, 120903, 120904, 120905, 120906, 120907, 120908, 120909, 120910, 120911, 120912 were selected from the ORFs having the nucleotide sequence represented by SEQ ID NO: 78756.

The probes having the nucleotide sequence represented by SEQ ID NOS: 120913, 120914, 120915, 120916, 120917, 120918, 120919, 120920, 120921, 120922, 120923 were selected from the ORFs having the nucleotide sequence represented by SEQ ID NO: 78757.

The probes having the nucleotide sequence represented by SEQ ID NOS: 120924, 120925, 120926, 120927, 120928, 120929, 120930, 120931, 120932, 120933, 120934 were selected from the ORFs having the nucleotide sequence represented by SEQ ID NO: 78758.

The probes having the nucleotide sequence represented by SEQ ID NOS: 120935, 120936, 120937, 120938, 120939, 120940, 120941, 120942, 120943, 120944, 120945 were selected from the ORFs having the nucleotide sequence represented by SEQ ID NO: 78759.

The probes having the nucleotide sequence represented by SEQ ID NOS: 120946, 120947, 120948, 120949, 120950, 120951, 120952, 120953, 120954, 120955, 120956 were selected from the ORFs having the nucleotide sequence represented by SEQ ID NO: 78760.

The probes having the nucleotide sequence represented by SEQ ID NOS: 120957, 120958, 120959, 120960, 120961, 120962, 120963, 120964, 120965, 120966, 120967 were selected from the ORFs having the nucleotide sequence represented by SEQ ID NO: 78761.

The probes having the nucleotide sequence represented by SEQ ID NOS: 120968, 120969, 120970, 120971, 120972, 120973, 120974, 120975, 120976, 120977, 120978 were selected from the ORFs having the nucleotide sequence represented by SEQ ID NO: 78762.

The probes having the nucleotide sequence represented by SEQ ID NOS: 120979, 120980, 120981, 120982, 120983, 120984, 120985, 120986, 120987, 120988, 120989 were selected from the ORFs having the nucleotide sequence represented by SEQ ID NO: 78763.

The probes having the nucleotide sequence represented by SEQ ID NOS: 120990, 120991, 120992, 120993, 120994, 120995, 120996, 120997, 120998, 120999, 121000 were selected from the ORFs having the nucleotide sequence represented by SEQ ID NO: 78764.

The probes having the nucleotide sequence represented by SEQ ID NOS: 121001, 121002, 121003, 121004, 121005, 121006, 121007, 121008, 121009, 121010, 121011 were selected from the ORFs having the nucleotide sequence represented by SEQ ID NO: 78765.

The probes having the nucleotide sequence represented by SEQ ID NOS: 121012, 121013, 121014, 121015, 121016, 121017, 121018, 121019, 121020, 121021, 121022 were selected from the ORFs having the nucleotide sequence represented by SEQ ID NO: 78766.

The probes having the nucleotide sequence represented by SEQ ID NOS: 121023, 121024, 121025, 121026, 121027, 121028, 121029, 121030, 121031, 121032, 121033 were selected from the ORFs having the nucleotide sequence represented by SEQ ID NO: 78767.

The probes having the nucleotide sequence represented by SEQ ID NOS: 121034, 121035, 121036, 121037, 121038, 121039, 121040, 121041, 121042, 121043, 121044 were selected from the ORFs having the nucleotide sequence represented by SEQ ID NO: 78768.

The probes having the nucleotide sequence represented by SEQ ID NOS: 121045, 121046, 121047, 121048, 121049, 121050, 121051, 121052, 121053, 121054, 121055 were selected from the ORFs having the nucleotide sequence represented by SEQ ID NO: 78769.

The probes having the nucleotide sequence represented by SEQ ID NOS: 121056, 121057, 121058, 121059, 121060, 121061, 121062, 121063, 121064, 121065, 121066 were selected from the ORFs having the nucleotide sequence represented by SEQ ID NO: 78770.

The probes having the nucleotide sequence represented by SEQ ID NOS: 121067, 121068, 121069, 121070, 121071, 121072, 121073, 121074, 121075, 121076, 121077 were selected from the ORFs having the nucleotide sequence represented by SEQ ID NO: 78771.

The probes having the nucleotide sequence represented by SEQ ID NOS: 121078, 121079, 121080, 121081, 121082, 121083, 121084, 121085, 121086, 121087, 121088, 121089, 121090, 121091, 121092, 121093, 121094, 121095, 121096, 121097, 121098, 121099 were selected from the ORFs having the nucleotide sequence represented by SEQ ID NO: 78772.

The probes having the nucleotide sequence represented by SEQ ID NOS: 121100, 121101, 121102, 121103, 121104, 121105, 121106, 121107, 121108, 121109, 121110 were selected from the ORFs having the nucleotide sequence represented by SEQ ID NO: 78773.

The probes having the nucleotide sequence represented by SEQ ID NOS: 121111, 121112, 121113, 121114, 121115, 121116, 121117, 121118, 121119, 121120, 121121 were selected from the ORFs having the nucleotide sequence represented by SEQ ID NO: 78774.

The probes having the nucleotide sequence represented by SEQ ID NOS: 121122, 121123, 121124, 121125, 121126, 121127, 121128, 121129, 121130, 121131, 121132 were selected from the ORFs having the nucleotide sequence represented by SEQ ID NO: 78775.

The probes having the nucleotide sequence represented by SEQ ID NOS: 121133, 121134, 121135, 121136, 121137, 121138, 121139, 121140, 121141, 121142, 121143 were selected from the ORFs having the nucleotide sequence represented by SEQ ID NO: 78776.

The probes having the nucleotide sequence represented by SEQ ID NOS: 121144, 121145, 121146, 121147, 121148, 121149, 121150, 121151, 121152, 121153, 121154 were selected from the ORFs having the nucleotide sequence represented by SEQ ID NO: 78777.

The probes having the nucleotide sequence represented by SEQ ID NOS: 121155, 121156, 121157, 121158, 121159, 121160, 121161, 121162, 121163, 121164, 121165 were selected from the ORFs having the nucleotide sequence represented by SEQ ID NO: 78778.

The probes having the nucleotide sequence represented by SEQ ID NOS: 121166, 121167, 121168, 121169, 121170, 121171, 121172, 121173, 121174, 121175, 121176 were selected from the ORFs having the nucleotide sequence represented by SEQ ID NO: 78779.

The probes having the nucleotide sequence represented by SEQ ID NOS: 121177, 121178, 121179, 121180, 121181, 121182, 121183, 121184, 121185, 121186, 121187 were selected from the ORFs having the nucleotide sequence represented by SEQ ID NO: 78780.

The probes having the nucleotide sequence represented by SEQ ID NOS: 121188, 121189, 121190, 121191, 121192, 121193, 121194, 121195, 121196, 121197, 121198 were selected from the ORFs having the nucleotide sequence represented by SEQ ID NO: 78781.

The probes having the nucleotide sequence represented by SEQ ID NOS: 121199, 121200, 121201, 121202, 121203, 121204, 121205, 121206, 121207, 121208, 121209 were selected from the ORFs having the nucleotide sequence represented by SEQ ID NO: 78782.

The probes having the nucleotide sequence represented by SEQ ID NOS: 121210, 121211, 121212, 121213, 121214, 121215, 121216, 121217, 121218, 121219, 121220 were selected from the ORFs having the nucleotide sequence represented by SEQ ID NO: 78783.

The probes having the nucleotide sequence represented by SEQ ID NOS: 121221, 121222, 121223, 121224, 121225, 121226, 121227, 121228, 121229, 121230, 121231 were selected from the ORFs having the nucleotide sequence represented by SEQ ID NO: 78784.

The probes having the nucleotide sequence represented by SEQ ID NOS: 121232, 121233, 121234, 121235, 121236, 121237, 121238, 121239, 121240, 121241, 121242 were selected from the ORFs having the nucleotide sequence represented by SEQ ID NO: 78785.

The probes having the nucleotide sequence represented by SEQ ID NOS: 121243, 121244, 121245, 121246, 121247, 121248, 121249, 121250, 121251, 121252, 121253 were selected from the ORFs having the nucleotide sequence represented by SEQ ID NO: 78786.

The probes having the nucleotide sequence represented by SEQ ID NOS: 121254, 121255, 121256, 121257, 121258, 121259, 121260, 121261, 121262, 121263, 121264 were selected from the ORFs having the nucleotide sequence represented by SEQ ID NO: 78787.

The probes having the nucleotide sequence represented by SEQ ID NOS: 121265, 121266, 121267, 121268, 121269, 121270, 121271, 121272, 121273, 121274, 121275 were selected from the ORFs having the nucleotide sequence represented by SEQ ID NO: 78788.

The probes having the nucleotide sequence represented by SEQ ID NOS: 121276, 121277, 121278, 121279, 121280, 121281, 121282, 121283, 121284, 121285, 121286 were selected from the ORFs having the nucleotide sequence represented by SEQ ID NO: 78789.

The probes having the nucleotide sequence represented by SEQ ID NOS: 121287, 121288, 121289, 121290, 121291, 121292, 121293, 121294, 121295, 121296, 121297 were selected from the ORFs having the nucleotide sequence represented by SEQ ID NO: 78790.

The probes having the nucleotide sequence represented by SEQ ID NOS: 121298, 121299, 121300, 121301, 121302, 121303, 121304, 121305, 121306, 121307, 121308 were selected from the ORFs having the nucleotide sequence represented by SEQ ID NO: 78791.

The probes having the nucleotide sequence represented by SEQ ID NOS: 121309, 121310, 121311, 121312, 121313, 121314, 121315, 121316, 121317, 121318, 121319 were selected from the ORFs having the nucleotide sequence represented by SEQ ID NO: 78792.

The probes having the nucleotide sequence represented by SEQ ID NOS: 121320, 121321, 121322, 121323, 121324, 121325, 121326, 121327, 121328, 121329, 121330 were selected from the ORFs having the nucleotide sequence represented by SEQ ID NO: 78793.

The probes having the nucleotide sequence represented by SEQ ID NOS: 121331, 121332, 121333, 121334, 121335, 121336, 121337, 121338, 121339, 121340, 121341 were selected from the ORFs having the nucleotide sequence represented by SEQ ID NO: 78794.

The probes having the nucleotide sequence represented by SEQ ID NOS: 121342, 121343, 121344, 121345, 121346, 121347, 121348, 121349, 121350, 121351, 121352 were selected from the ORFs having the nucleotide sequence represented by SEQ ID NO: 78795.

The probes having the nucleotide sequence represented by SEQ ID NOS: 121353, 121354, 121355, 121356, 121357, 121358, 121359, 121360, 121361, 121362, 121363 were selected from the ORFs having the nucleotide sequence represented by SEQ ID NO: 78796.

The probes having the nucleotide sequence represented by SEQ ID NOS: 121364, 121365, 121366, 121367, 121368, 121369, 121370, 121371, 121372, 121373, 121374 were selected from the ORFs having the nucleotide sequence represented by SEQ ID NO: 78797.

The probes having the nucleotide sequence represented by SEQ ID NOS: 121375, 121376, 121377, 121378, 121379, 121380, 121381, 121382, 121383, 121384, 121385 were selected from the ORFs having the nucleotide sequence represented by SEQ ID NO: 78798.

The probes having the nucleotide sequence represented by SEQ ID NOS: 121386, 121387, 121388, 121389, 121390, 121391, 121392, 121393, 121394, 121395, 121396 were selected from he ORFs having the nucleotide sequence represented by SEQ ID NO: 78799.

The probes having the nucleotide sequence represented by SEQ ID NOS: 121397, 121398, 121399, 121400, 121401, 121402, 121403, 121404, 121405, 121406, 121407 were selected from the ORFs having the nucleotide sequence represented by SEQ ID NO: 78800.

The probes having the nucleotide sequence represented by SEQ ID NOS: 121408, 121409, 121410, 121411, 121412, 121413, 121414, 121415, 121416, 121417, 121418 were selected from the ORFs having the nucleotide sequence represented by SEQ ID NO: 78801.

The probes having the nucleotide sequence represented by SEQ ID NOS: 121419, 121420, 121421, 121422, 121423, 121424, 121425, 121426, 121427, 121428, 121429 were selected from the ORFs having the nucleotide sequence represented by SEQ ID NO: 78802.

The probes having the nucleotide sequence represented by SEQ ID NOS: 121430, 121431, 121432, 121433, 121434, 121435, 121436, 121437, 121438, 121439, 121440 were selected from the ORFs having the nucleotide sequence represented by SEQ ID NO: 78803.

The probes having the nucleotide sequence represented by SEQ ID NOS: 121441, 121442, 121443, 121444, 121445, 121446, 121447, 121448, 121449, 121450, 121451 were selected from the ORFs having the nucleotide sequence represented by SEQ ID NO: 78804.

The probes having the nucleotide sequence represented by SEQ ID NOS: 121452, 121453, 121454, 121455, 121456, 121457, 121458, 121459, 121460, 121461, 121462 were selected from the ORFs having the nucleotide sequence represented by SEQ ID NO: 78805.

The probes having the nucleotide sequence represented by SEQ ID NOS: 121463, 121464, 121465, 121466, 121467, 121468, 121469, 121470, 121471, 121472, 121473 were selected from the ORFs having the nucleotide sequence represented by SEQ ID NO: 78806.

The probes having the nucleotide sequence represented by SEQ ID NOS: 121474, 121475, 121476, 121477, 121478, 121479, 121480, 121481, 121482, 121483, 121484 were selected from the ORFs having the nucleotide sequence represented by SEQ ID NO: 78807.

The probes having the nucleotide sequence represented by SEQ ID NOS: 121485, 121486, 121487, 121488, 121489, 121490, 121491, 121492, 121493, 121494, 121495 were selected from the ORFs having the nucleotide sequence represented by SEQ ID NO: 78808.

The probes having the nucleotide sequence represented by SEQ ID NOS: 121496, 121497, 121498, 121499, 121500, 121501, 121502, 121503, 121504, 121505, 121506 were selected from the ORFs having the nucleotide sequence represented by SEQ ID NO: 78809.

The probes having the nucleotide sequence represented by SEQ ID NOS: 121507, 121508, 121509, 121510, 121511, 121512, 121513, 121514, 121515, 121516, 121517 were selected from the ORFs having the nucleotide sequence represented by SEQ ID NO: 78810.

The probes having the nucleotide sequence represented by SEQ ID NOS: 121518, 121519, 121520, 121521, 121522, 121523, 121524, 121525, 121526, 121527, 121528 were selected from the ORFs having the nucleotide sequence represented by SEQ ID NO: 78811.

The probes having the nucleotide sequence represented by SEQ ID NOS: 121529, 121530, 121531, 121532, 121533, 121534, 121535, 121536, 121537, 121538, 121539 were selected from the ORFs having the nucleotide sequence represented by SEQ ID NO: 78812.

The probes having the nucleotide sequence represented by SEQ ID NOS: 121540, 121541, 121542, 121543, 121544, 121545, 121546, 121547, 121548, 121549, 121550 were selected from the ORFs having the nucleotide sequence represented by SEQ ID NO: 78813.

The probes having the nucleotide sequence represented by SEQ ID NOS: 121551, 121552, 121553, 121554, 121555, 121556, 121557, 121558, 121559, 121560, 121561 were selected from the ORFs having the nucleotide sequence represented by SEQ ID NO: 78814.

The probes having the nucleotide sequence represented by SEQ ID NOS: 121562, 121563, 121564, 121565, 121566, 121567, 121568, 121569, 121570, 121571, 121572 were selected from the ORFs having the nucleotide sequence represented by SEQ ID NO: 78815.

The probes having the nucleotide sequence represented by SEQ ID NOS: 121573, 121574, 121575, 121576, 121577, 121578, 121579, 121580, 121581, 121582, 121583 were selected from the ORFs having the nucleotide sequence represented by SEQ ID NO: 78816.

The probes having the nucleotide sequence represented by SEQ ID NOS: 121584, 121585, 121586, 121587, 121588, 121589, 121590, 121591, 121592, 121593, 121594 were selected from the ORFs having the nucleotide sequence represented by SEQ ID NO: 78817.

The probes having the nucleotide sequence represented by SEQ ID NOS: 121595, 121596, 121597, 121598, 121599, 121600, 121601, 121602, 121603, 121604, 121605 were selected from the ORFs having the nucleotide sequence represented by SEQ ID NO: 78818.

The probes having the nucleotide sequence represented by SEQ ID NOS: 121606, 121607, 121608, 121609, 121610, 121611, 121612, 121613, 121614, 121615, 121616 were selected from the ORFs having the nucleotide sequence represented by SEQ ID NO: 78819.

The probes having the nucleotide sequence represented by SEQ ID NOS: 121617, 121618, 121619, 121620, 121621, 121622, 121623, 121624, 121625, 121626, 121627 were selected from the ORFs having the nucleotide sequence represented by SEQ ID NO: 78820.

The probes having the nucleotide sequence represented by SEQ ID NOS: 121628, 121629, 121630, 121631, 121632, 121633, 121634, 121635, 121636, 121637, 121638 were selected from the ORFs having the nucleotide sequence represented by SEQ ID NO: 78821.

The probes having the nucleotide sequence represented by SEQ ID NOS: 121639, 121640, 121641, 121642, 121643, 121644, 121645, 121646, 121647, 121648, 121649 were selected from the ORFs having the nucleotide sequence represented by SEQ ID NO: 78822.

The probes having the nucleotide sequence represented by SEQ ID NOS: 121650, 121651, 121652, 121653, 121654, 121655, 121656, 121657, 121658, 121659, 121660 were selected from the ORFs having the nucleotide sequence represented by SEQ ID NO: 78823.

The probes having the nucleotide sequence represented by SEQ ID NOS: 121661, 121662, 121663, 121664, 121665, 121666, 121667, 121668, 121669, 121670, 121671 were selected from the ORFs having the nucleotide sequence represented by SEQ ID NO: 78824.

The probes having the nucleotide sequence represented by SEQ ID NOS: 121672, 121673, 121674, 121675, 121676, 121677, 121678, 121679, 121680, 121681, 121682 were selected from the ORFs having the nucleotide sequence represented by SEQ ID NO: 78825.

The probes having the nucleotide sequence represented by SEQ ID NOS: 121683, 121684, 121685, 121686, 121687, 121688, 121689, 121690, 121691, 121692, 121693 were selected from the ORFs having the nucleotide sequence represented by SEQ ID NO: 78826.

The probes having the nucleotide sequence represented by SEQ ID NOS: 121694, 121695, 121696, 121697, 121698, 121699, 121700, 121701, 121702, 121703, 121704 were selected from the ORFs having the nucleotide sequence represented by SEQ ID NO: 78827.

The probes having the nucleotide sequence represented by SEQ ID NOS: 121705, 121706, 121707, 121708, 121709, 121710, 121711, 121712, 121713, 121714, 121715 were selected from the ORFs having the nucleotide sequence represented by SEQ ID NO: 78828.

The probes having the nucleotide sequence represented by SEQ ID NOS: 121716, 121717, 121718, 121719, 121720, 121721, 121722, 121723, 121724, 121725, 121726 were selected from the ORFs having the nucleotide sequence represented by SEQ ID NO: 78829.

The probes having the nucleotide sequence represented by SEQ ID NOS: 121727, 121728, 121729, 121730, 121731, 121732, 121733, 121734, 121735, 121736, 121737 were selected from the ORFs having the nucleotide sequence represented by SEQ ID NO: 78830.

The probes having the nucleotide sequence represented by SEQ ID NOS: 121738, 121739, 121740, 121741, 121742, 121743, 121744, 121745, 121746, 121747, 121748 were selected from the ORFs having the nucleotide sequence represented by SEQ ID NO: 78831.

The probes having the nucleotide sequence represented by SEQ ID NOS: 121749, 121750, 121751, 121752, 121753, 121754, 121755, 121756, 121757, 121758, 121759 were selected from the ORFs having the nucleotide sequence represented by SEQ ID NO: 78832.

The probes having the nucleotide sequence represented by SEQ ID NOS: 121760, 121761, 121762, 121763, 121764, 121765, 121766, 121767, 121768, 121769, 121770 were selected from the ORFs having the nucleotide sequence represented by SEQ ID NO: 78833.

The probes having the nucleotide sequence represented by SEQ ID NOS: 121771, 121772, 121773, 121774, 121775, 121776, 121777, 121778, 121779, 121780, 121781 were selected from the ORFs having the nucleotide sequence represented by SEQ ID NO: 78834.

The probes having the nucleotide sequence represented by SEQ ID NOS: 121782, 121783, 121784, 121785, 121786, 121787, 121788, 121789, 121790, 121791, 121792 were selected from the ORFs having the nucleotide sequence represented by SEQ ID NO: 78835.

The probes having the nucleotide sequence represented by SEQ ID NOS: 121793, 121794, 121795, 121796, 121797, 121798, 121799, 121800, 121801, 121802, 121803 were selected from the ORFs having the nucleotide sequence represented by SEQ ID NO: 78836.

The probes having the nucleotide sequence represented by SEQ ID NOS: 121804, 121805, 121806, 121807, 121808, 121809, 121810, 121811, 121812, 121813, 121814 were selected from the ORFs having the nucleotide sequence represented by SEQ ID NO: 78837.

The probes having the nucleotide sequence represented by SEQ ID NOS: 121815, 121816, 121817, 121818, 121819, 121820, 121821, 121822, 121823, 121824, 121825 were selected from the ORFs having the nucleotide sequence represented by SEQ ID NO: 78838.

The probes having the nucleotide sequence represented by SEQ ID NOS: 121826, 121827, 121828, 121829, 121830, 121831, 121832, 121833, 121834, 121835, 121836, 121837, 121838, 121839, 121840, 121841, 121842, 121843, 121844, 121845, 121846, 121847 were selected from the ORFs having the nucleotide sequence represented by SEQ ID NO: 78839.

The probes having the nucleotide sequence represented by SEQ ID NOS: 121848, 121849, 121850, 121851, 121852, 121853, 121854, 121855, 121856, 121857, 121858 were selected from the ORFs having the nucleotide sequence represented by SEQ ID NO: 78840.

The probes having the nucleotide sequence represented by SEQ ID NOS: 121859, 121860, 121861, 121862, 121863, 121864, 121865, 121866, 121867, 121868, 121869 were selected from the ORFs having the nucleotide sequence represented by SEQ ID NO: 78841.

The probes having the nucleotide sequence represented by SEQ ID NOS: 121870, 121871, 121872, 121873, 121874, 121875, 121876, 121877, 121878, 121879, 121880 were selected from the ORFs having the nucleotide sequence represented by SEQ ID NO: 78842.

The probes having the nucleotide sequence represented by SEQ ID NOS: 121881, 121882, 121883, 121884, 121885, 121886, 121887, 121888, 121889, 121890, 121891 were selected from the ORFs having the nucleotide sequence represented by SEQ ID NO: 78843.

The probes having the nucleotide sequence represented by SEQ ID NOS: 121892, 121893, 121894, 121895, 121896, 121897, 121898, 121899, 121900, 121901, 121902 were selected from the ORFs having the nucleotide sequence represented by SEQ ID NO: 78844.

The probes having the nucleotide sequence represented by SEQ ID NOS: 121903, 121904, 121905, 121906, 121907, 121908, 121909, 121910, 121911, 121912, 121913 were selected from the ORFs having the nucleotide sequence represented by SEQ ID NO: 78845.

The probes having the nucleotide sequence represented by SEQ ID NOS: 121914, 121915, 121916, 121917, 121918, 121919, 121920, 121921, 121922, 121923, 121924 were selected from the ORFs having the nucleotide sequence represented by SEQ ID NO: 78846.

The probes having the nucleotide sequence represented by SEQ ID NOS: 121925, 121926, 121927, 121928, 121929, 121930, 121931, 121932, 121933, 121934, 121935 were selected from the ORFs having the nucleotide sequence represented by SEQ ID NO: 78847.

The probes having the nucleotide sequence represented by SEQ ID NOS: 121936, 121937, 121938, 121939, 121940, 121941, 121942, 121943, 121944, 121945, 121946 were selected from the ORFs having the nucleotide sequence represented by SEQ ID NO: 78848.

The probes having the nucleotide sequence represented by SEQ ID NOS: 121947, 121948, 121949, 121950, 121951, 121952, 121953, 121954, 121955, 121956, 121957 were selected from the ORFs having the nucleotide sequence represented by SEQ ID NO: 78849.

The probes having the nucleotide sequence represented by SEQ ID NOS: 121958, 121959, 121960, 121961, 121962, 121963, 121964, 121965, 121966, 121967, 121968 were selected from the ORFs having the nucleotide sequence represented by SEQ ID NO: 78850.

The probes having the nucleotide sequence represented by SEQ ID NOS: 121969, 121970, 121971, 121972, 121973, 121974, 121975, 121976, 121977, 121978, 121979 were selected from the ORFs having the nucleotide sequence represented by SEQ ID NO: 78851.

The probes having the nucleotide sequence represented by SEQ ID NOS: 121980, 121981, 121982, 121983, 121984, 121985, 121986, 121987, 121988, 121989, 121990 were selected from the ORFs having the nucleotide sequence represented by SEQ ID NO: 78852.

The probes having the nucleotide sequence represented by SEQ ID NOS: 121991, 121992, 121993, 121994, 121995, 121996, 121997, 121998, 121999, 122000, 122001 were selected from the ORFs having the nucleotide sequence represented by SEQ ID NO: 78853.

The probes having the nucleotide sequence represented by SEQ ID NOS: 122002, 122003, 122004, 122005, 122006, 122007, 122008, 122009, 122010, 122011, 122012 were selected from the ORFs having the nucleotide sequence represented by SEQ ID NO: 78854.

The probes having the nucleotide sequence represented by SEQ ID NOS: 122013, 122014, 122015, 122016, 122017, 122018, 122019, 122020, 122021, 122022, 122023 were selected from the ORFs having the nucleotide sequence represented by SEQ ID NO: 78855.

The probes having the nucleotide sequence represented by SEQ ID NOS: 122024, 122025, 122026, 122027, 122028, 122029, 122030, 122031, 122032, 122033, 122034 were selected from the ORFs having the nucleotide sequence represented by SEQ ID NO: 78856.

The probes having the nucleotide sequence represented by SEQ ID NOS: 122035, 122036, 122037, 122038, 122039, 122040, 122041, 122042, 122043, 122044, 122045 were selected from the ORFs having the nucleotide sequence represented by SEQ ID NO: 78857.

The probes having the nucleotide sequence represented by SEQ ID NOS: 122046, 122047, 122048, 122049, 122050, 122051, 122052, 122053, 122054, 122055, 122056 were selected from the ORFs having the nucleotide sequence represented by SEQ ID NO: 78858.

The probes having the nucleotide sequence represented by SEQ ID NOS: 122057, 122058, 122059, 122060, 122061, 122062, 122063, 122064, 122065, 122066, 122067 were selected from the ORFs having the nucleotide sequence represented by SEQ ID NO: 78859.

The probes having the nucleotide sequence represented by SEQ ID NOS: 122068, 122069, 122070, 122071, 122072, 122073, 122074, 122075, 122076, 122077, 122078 were selected from the ORFs having the nucleotide sequence represented by SEQ ID NO: 78860.

The probes having the nucleotide sequence represented by SEQ ID NOS: 122079, 122080, 122081, 122082, 122083, 122084, 122085, 122086, 122087, 122088, 122089 were selected from the ORFs having the nucleotide sequence represented by SEQ ID NO: 78861.

The probes having the nucleotide sequence represented by SEQ ID NOS: 122090, 122091, 122092, 122093, 122094, 122095, 122096, 122097, 122098, 122099, 122100 were selected from the ORFs having the nucleotide sequence represented by SEQ ID NO: 78862.

The probes having the nucleotide sequence represented by SEQ ID NOS: 122101, 122102, 122103, 122104, 122105, 122106, 122107, 122108, 122109, 122110, 122111 were selected from the ORFs having the nucleotide sequence represented by SEQ ID NO: 78863.

The probes having the nucleotide sequence represented by SEQ ID NOS: 122112, 122113, 122114, 122115, 122116, 122117, 122118, 122119, 122120, 122121, 122122 were selected from the ORFs having the nucleotide sequence represented by SEQ ID NO: 78864.

The probes having the nucleotide sequence represented by SEQ ID NOS: 122123, 122124, 122125, 122126, 122127, 122128, 122129, 122130, 122131, 122132, 122133 were selected from the ORFs having the nucleotide sequence represented by SEQ ID NO: 78865.

The probes having the nucleotide sequence represented by SEQ ID NOS: 122134, 122135, 122136, 122137, 122138, 122139, 122140, 122141, 122142, 122143, 122144 were selected from the ORFs having the nucleotide sequence represented by SEQ ID NO: 78866.

The probes having the nucleotide sequence represented by SEQ ID NOS: 122145, 122146, 122147, 122148, 122149, 122150, 122151, 122152, 122153, 122154, 122155 were selected from the ORFs having the nucleotide sequence represented by SEQ ID NO: 78867.

The probes having the nucleotide sequence represented by SEQ ID NOS: 122156, 122157, 122158, 122159, 122160, 122161, 122162, 122163, 122164, 122165, 122166 were selected from the ORFs having the nucleotide sequence represented by SEQ ID NO: 78868.

The probes having the nucleotide sequence represented by SEQ ID NOS: 122167, 122168, 122169, 122170, 122171, 122172, 122173, 122174, 122175, 122176, 122177 were selected from the ORFs having the nucleotide sequence represented by SEQ ID NO: 78869.

The probes having the nucleotide sequence represented by SEQ ID NOS: 122178, 122179, 122180, 122181, 122182, 122183, 122184, 122185, 122186, 122187, 122188 were selected from the ORFs having the nucleotide sequence represented by SEQ ID NO: 78870.

The probes having the nucleotide sequence represented by SEQ ID NOS: 122189, 122190, 122191, 122192, 122193, 122194, 122195, 122196, 122197, 122198, 122199 were selected from the ORFs having the nucleotide sequence represented by SEQ ID NO: 78871.

The probes having the nucleotide sequence represented by SEQ ID NOS: 122200, 122201, 122202, 122203, 122204, 122205, 122206, 122207, 122208, 122209, 122210 were selected from the ORFs having the nucleotide sequence represented by SEQ ID NO: 78872.

The probes having the nucleotide sequence represented by SEQ ID NOS: 122211, 122212, 122213, 122214, 122215, 122216, 122217, 122218, 122219, 122220, 122221 were selected from the ORFs having the nucleotide sequence represented by SEQ ID NO: 78873.

The probes having the nucleotide sequence represented by SEQ ID NOS: 122222, 122223, 122224, 122225, 122226, 122227, 122228, 122229, 122230, 122231, 122232 were selected from the ORFs having the nucleotide sequence represented by SEQ ID NO: 78874.

The probes having the nucleotide sequence represented by SEQ ID NOS: 122233, 122234, 122235, 122236, 122237, 122238, 122239, 122240, 122241, 122242, 122243 were selected from the ORFs having the nucleotide sequence represented by SEQ ID NO: 78875.

The probes having the nucleotide sequence represented by SEQ ID NOS: 122244, 122245, 122246, 122247, 122248, 122249, 122250, 122251, 122252, 122253, 122254 were selected from the ORFs having the nucleotide sequence represented by SEQ ID NO: 78876.

The probes having the nucleotide sequence represented by SEQ ID NOS: 122255, 122256, 122257, 122258, 122259, 122260, 122261, 122262, 122263, 122264, 122265 were selected from the ORFs having the nucleotide sequence represented by SEQ ID NO: 78877.

The probes having the nucleotide sequence represented by SEQ ID NOS: 122266, 122267, 122268, 122269, 122270, 122271, 122272, 122273, 122274, 122275, 122276 were selected from the ORFs having the nucleotide sequence represented by SEQ ID NO: 78878.

The probes having the nucleotide sequence represented by SEQ ID NOS: 122277, 122278, 122279, 122280, 122281, 122282, 122283, 122284, 122285, 122286, 122287 were selected from the ORFs having the nucleotide sequence represented by SEQ ID NO: 78879.

The probes having the nucleotide sequence represented by SEQ ID NOS: 122288, 122289, 122290, 122291, 122292, 122293, 122294, 122295, 122296, 122297, 122298 were selected from the ORFs having the nucleotide sequence represented by SEQ ID NO: 78880.

The probes having the nucleotide sequence represented by SEQ ID NOS: 122299, 122300, 122301, 122302, 122303, 122304, 122305, 122306, 122307, 122308, 122309 were selected from the ORFs having the nucleotide sequence represented by SEQ ID NO: 78881.

The probes having the nucleotide sequence represented by SEQ ID NOS: 122310, 122311, 122312, 122313, 122314, 122315, 122316, 122317, 122318, 122319, 122320 were selected from the ORFs having the nucleotide sequence represented by SEQ ID NO: 78882.

The probes having the nucleotide sequence represented by SEQ ID NOS: 122321, 122322, 122323, 122324, 122325, 122326, 122327, 122328, 122329, 122330, 122331 were selected from the ORFs having the nucleotide sequence represented by SEQ ID NO: 78883.

The probes having the nucleotide sequence represented by SEQ ID NOS: 122332, 122333, 122334, 122335, 122336, 122337, 122338, 122339, 122340, 122341, 122342 were selected from the ORFs having the nucleotide sequence represented by SEQ ID NO: 78884.

The probes having the nucleotide sequence represented by SEQ ID NOS: 122343, 122344, 122345, 122346, 122347, 122348, 122349, 122350, 122351, 122352, 122353 were selected from the ORFs having the nucleotide sequence represented by SEQ ID NO: 78885.

The probes having the nucleotide sequence represented by SEQ ID NOS: 122354, 122355, 122356, 122357, 122358, 122359, 122360, 122361, 122362, 122363, 122364 were selected from the ORFs having the nucleotide sequence represented by SEQ ID NO: 78886.

The probes having the nucleotide sequence represented by SEQ ID NOS: 122365, 122366, 122367, 122368, 122369, 122370, 122371, 122372, 122373, 122374, 122375 were selected from the ORFs having the nucleotide sequence represented by SEQ ID NO: 78887.

The probes having the nucleotide sequence represented by SEQ ID NOS: 122376, 122377, 122378, 122379, 122380, 122381, 122382, 122383, 122384, 122385, 122386 were selected from the ORFs having the nucleotide sequence represented by SEQ ID NO: 78888.

The probes having the nucleotide sequence represented by SEQ ID NOS: 122387, 122388, 122389, 122390, 122391, 122392, 122393, 122394, 122395, 122396, 122397 were selected from the ORFs having the nucleotide sequence represented by SEQ ID NO: 78889.

The probes having the nucleotide sequence represented by SEQ ID NOS: 122398, 122399, 122400, 122401, 122402, 122403, 122404, 122405, 122406, 122407, 122408 were selected from the ORFs having the nucleotide sequence represented by SEQ ID NO: 78890.

The probes having the nucleotide sequence represented by SEQ ID NOS: 122409, 122410, 122411, 122412, 122413, 122414, 122415, 122416, 122417, 122418, 122419 were selected from the ORFs having the nucleotide sequence represented by SEQ ID NO: 78891.

The probes having the nucleotide sequence represented by SEQ ID NOS: 122420, 122421, 122422, 122423, 122424, 122425, 122426, 122427, 122428, 122429, 122430 were selected from the ORFs having the nucleotide sequence represented by SEQ ID NO: 78892.

The probes having the nucleotide sequence represented by SEQ ID NOS: 122431, 122432, 122433, 122434, 122435, 122436, 122437, 122438, 122439, 122440, 122441 were selected from the ORFs having the nucleotide sequence represented by SEQ ID NO: 78893.

The probes having the nucleotide sequence represented by SEQ ID NOS: 122442, 122443, 122444, 122445, 122446, 122447, 122448, 122449, 122450, 122451, 122452 were selected from the ORFs having the nucleotide sequence represented by SEQ ID NO: 78894.

The probes having the nucleotide sequence represented by SEQ ID NOS: 122453, 122454, 122455, 122456, 122457, 122458, 122459, 122460, 122461, 122462, 122463 were selected from the ORFs having the nucleotide sequence represented by SEQ ID NO: 78895.

The probes having the nucleotide sequence represented by SEQ ID NOS: 122464, 122465, 122466, 122467, 122468, 122469, 122470, 122471, 122472, 122473, 122474 were selected from the ORFs having the nucleotide sequence represented by SEQ ID NO: 78896.

The probes having the nucleotide sequence represented by SEQ ID NOS: 122475, 122476, 122477, 122478, 122479, 122480, 122481, 122482, 122483, 122484, 122485 were selected from the ORFs having the nucleotide sequence represented by SEQ ID NO: 78897.

The probes having the nucleotide sequence represented by SEQ ID NOS: 122486, 122487, 122488, 122489, 122490, 122491, 122492, 122493, 122494, 122495, 122496 were selected from the ORFs having the nucleotide sequence represented by SEQ ID NO: 78898.

The probes having the nucleotide sequence represented by SEQ ID NOS: 122497, 122498, 122499, 122500, 122501, 122502, 122503, 122504, 122505, 122506, 122507 were selected from the ORFs having the nucleotide sequence represented by SEQ ID NO: 78899.

The probes having the nucleotide sequence represented by SEQ ID NOS: 122508, 122509, 122510, 122511, 122512, 122513, 122514, 122515, 122516, 122517, 122518 were selected from the ORFs having the nucleotide sequence represented by SEQ ID NO: 78900.

The probes having the nucleotide sequence represented by SEQ ID NOS: 122519, 122520, 122521, 122522, 122523, 122524, 122525, 122526, 122527, 122528, 122529 were selected from the ORFs having the nucleotide sequence represented by SEQ ID NO: 78901.

The probes having the nucleotide sequence represented by SEQ ID NOS: 122530, 122531, 122532, 122533, 122534, 122535, 122536, 122537, 122538, 122539, 122540 were selected from the ORFs having the nucleotide sequence represented by SEQ ID NO: 78902.

The probes having the nucleotide sequence represented by SEQ ID NOS: 122541, 122542, 122543, 122544, 122545, 122546, 122547, 122548, 122549, 122550, 122551 were selected from the ORFs having the nucleotide sequence represented by SEQ ID NO: 78903.

The probes having the nucleotide sequence represented by SEQ ID NOS: 122552, 122553, 122554, 122555, 122556, 122557, 122558, 122559, 122560, 122561, 122562 were selected from the ORFs having the nucleotide sequence represented by SEQ ID NO: 78904.

The probes having the nucleotide sequence represented by SEQ ID NOS: 122563, 122564, 122565, 122566, 122567, 122568, 122569, 122570, 122571, 122572, 122573 were selected from the ORFs having the nucleotide sequence represented by SEQ ID NO: 78905.

The probes having the nucleotide sequence represented by SEQ ID NOS: 122574, 122575, 122576, 122577, 122578, 122579, 122580, 122581, 122582, 122583, 122584 were selected from the ORFs having the nucleotide sequence represented by SEQ ID NO: 78906.

The probes having the nucleotide sequence represented by SEQ ID NOS: 122585, 122586, 122587, 122588, 122589, 122590, 122591, 122592, 122593, 122594, 122595 were selected from the ORFs having the nucleotide sequence represented by SEQ ID NO: 78907.

The probes having the nucleotide sequence represented by SEQ ID NOS: 122596, 122597, 122598, 122599, 122600, 122601, 122602, 122603, 122604, 122605, 122606 were selected from the ORFs having the nucleotide sequence represented by SEQ ID NO: 78908.

The probes having the nucleotide sequence represented by SEQ ID NOS: 122607, 122608, 122609, 122610, 122611, 122612, 122613, 122614, 122615, 122616, 122617 were selected from the ORFs having the nucleotide sequence represented by SEQ ID NO: 78909.

The probes having the nucleotide sequence represented by SEQ ID NOS: 122618, 122619, 122620, 122621, 122622, 122623, 122624, 122625, 122626, 122627, 122628 were selected from the ORFs having the nucleotide sequence represented by SEQ ID NO: 78910.

The probes having the nucleotide sequence represented by SEQ ID NOS: 122629, 122630, 122631, 122632, 122633, 122634, 122635, 122636, 122637, 122638, 122639 were selected from the ORFs having the nucleotide sequence represented by SEQ ID NO: 78911.

The probes having the nucleotide sequence represented by SEQ ID NOS: 122640, 122641, 122642, 122643, 122644, 122645, 122646, 122647, 122648, 122649, 122650 were selected from the ORFs having the nucleotide sequence represented by SEQ ID NO: 78912.

The probes having the nucleotide sequence represented by SEQ ID NOS: 122651, 122652, 122653, 122654, 122655, 122656, 122657, 122658, 122659, 122660, 122661 were selected from the ORFs having the nucleotide sequence represented by SEQ ID NO: 78913.

The probes having the nucleotide sequence represented by SEQ ID NOS: 122662, 122663, 122664, 122665, 122666, 122667, 122668, 122669, 122670, 122671, 122672 were selected from the ORFs having the nucleotide sequence represented by SEQ ID NO: 78914.

The probes having the nucleotide sequence represented by SEQ ID NOS: 122673, 122674, 122675, 122676, 122677, 122678, 122679, 122680, 122681, 122682, 122683 were selected from the ORFs having the nucleotide sequence represented by SEQ ID NO: 78915.

The probes having the nucleotide sequence represented by SEQ ID NOS: 122684, 122685, 122686, 122687, 122688, 122689, 122690, 122691, 122692, 122693, 122694 were selected from the ORFs having the nucleotide sequence represented by SEQ ID NO: 78916.

The probes having the nucleotide sequence represented by SEQ ID NOS: 122695, 122696, 122697, 122698, 122699, 122700, 122701, 122702, 122703, 122704, 122705 were selected from the ORFs having the nucleotide sequence represented by SEQ ID NO: 78917.

The probes having the nucleotide sequence represented by SEQ ID NOS: 122706, 122707, 122708, 122709, 122710, 122711, 122712, 122713, 122714, 122715, 122716 were selected from the ORFs having the nucleotide sequence represented by SEQ ID NO: 78918.

The probes having the nucleotide sequence represented by SEQ ID NOS: 122717, 122718, 122719, 122720, 122721, 122722, 122723, 122724, 122725, 122726, 122727 were selected from the ORFs having the nucleotide sequence represented by SEQ ID NO: 78919.

The probes having the nucleotide sequence represented by SEQ ID NOS: 122728, 122729, 122730, 122731, 122732, 122733, 122734, 122735, 122736, 122737, 122738 were selected from the ORFs having the nucleotide sequence represented by SEQ ID NO: 78920.

The probes having the nucleotide sequence represented by SEQ ID NOS: 122739, 122740, 122741, 122742, 122743, 122744, 122745, 122746, 122747, 122748, 122749 were selected from the ORFs having the nucleotide sequence represented by SEQ ID NO: 78921.

The probes having the nucleotide sequence represented by SEQ ID NOS: 122750, 122751, 122752, 122753, 122754, 122755, 122756, 122757, 122758, 122759, 122760 were selected from the ORFs having the nucleotide sequence represented by SEQ ID NO: 78922.

The probes having the nucleotide sequence represented by SEQ ID NOS: 122761, 122762, 122763, 122764, 122765, 122766, 122767, 122768, 122769, 122770, 122771 were selected from the ORFs having the nucleotide sequence represented by SEQ ID NO: 78923.

The probes having the nucleotide sequence represented by SEQ ID NOS: 122772, 122773, 122774, 122775, 122776, 122777, 122778, 122779, 122780, 122781, 122782 were selected from the ORFs having the nucleotide sequence represented by SEQ ID NO: 78924.

The probes having the nucleotide sequence represented by SEQ ID NOS: 122783, 122784, 122785, 122786, 122787, 122788, 122789, 122790, 122791, 122792, 122793 were selected from the ORFs having the nucleotide sequence represented by SEQ ID NO: 78925.

The probes having the nucleotide sequence represented by SEQ ID NOS: 122794, 122795, 122796, 122797, 122798, 122799, 122800, 122801, 122802, 122803, 122804 were selected from the ORFs having the nucleotide sequence represented by SEQ ID NO: 78926.

The probes having the nucleotide sequence represented by SEQ ID NOS: 122805, 122806, 122807, 122808, 122809, 122810, 122811, 122812, 122813, 122814, 122815 were selected from the ORFs having the nucleotide sequence represented by SEQ ID NO: 78927.

The probes having the nucleotide sequence represented by SEQ ID NOS: 122816, 122817, 122818, 122819, 122820, 122821, 122822, 122823, 122824, 122825, 122826 were selected from the ORFs having the nucleotide sequence represented by SEQ ID NO: 78928.

The probes having the nucleotide sequence represented by SEQ ID NOS: 122827, 122828, 122829, 122830, 122831, 122832, 122833, 122834, 122835, 122836, 122837 were selected from the ORFs having the nucleotide sequence represented by SEQ ID NO: 78929.

The probes having the nucleotide sequence represented by SEQ ID NOS: 122838, 122839, 122840, 122841, 122842, 122843, 122844, 122845, 122846, 122847, 122848 were selected from the ORFs having the nucleotide sequence represented by SEQ ID NO: 78930.

The probes having the nucleotide sequence represented by SEQ ID NOS: 122849, 122850, 122851, 122852, 122853, 122854, 122855, 122856, 122857, 122858, 122859 were selected from the ORFs having the nucleotide sequence represented by SEQ ID NO: 78931.

The probes having the nucleotide sequence represented by SEQ ID NOS: 122860, 122861, 122862, 122863, 122864, 122865, 122866, 122867, 122868, 122869, 122870 were selected from the ORFs having the nucleotide sequence represented by SEQ ID NO: 78932.

The probes having the nucleotide sequence represented by SEQ ID NOS: 122871, 122872, 122873, 122874, 122875, 122876, 122877, 122878, 122879, 122880, 122881 were selected from the ORFs having the nucleotide sequence represented by SEQ ID NO: 78933.

The probes having the nucleotide sequence represented by SEQ ID NOS: 122882, 122883, 122884, 122885, 122886, 122887, 122888, 122889, 122890, 122891, 122892 were selected from the ORFs having the nucleotide sequence represented by SEQ ID NO: 78934.

The probes having the nucleotide sequence represented by SEQ ID NOS: 122893, 122894, 122895, 122896, 122897, 122898, 122899, 122900, 122901, 122902, 122903 were selected from the ORFs having the nucleotide sequence represented by SEQ ID NO: 78935.

The probes having the nucleotide sequence represented by SEQ ID NOS: 122904, 122905, 122906, 122907, 122908, 122909, 122910, 122911, 122912, 122913, 122914 were selected from the ORFs having the nucleotide sequence represented by SEQ ID NO: 78936.

The probes having the nucleotide sequence represented by SEQ ID NOS: 122915, 122916, 122917, 122918, 122919, 122920, 122921, 122922, 122923, 122924, 122925 were selected from the ORFs having the nucleotide sequence represented by SEQ ID NO: 78937.

The probes having the nucleotide sequence represented by SEQ ID NOS: 122926, 122927, 122928, 122929, 122930, 122931, 122932, 122933, 122934, 122935, 122936 were selected from the ORFs having the nucleotide sequence represented by SEQ ID NO: 78938.

The probes having the nucleotide sequence represented by SEQ ID NOS: 122937, 122938, 122939, 122940, 122941, 122942, 122943, 122944, 122945, 122946, 122947 were selected from the ORFs having the nucleotide sequence represented by SEQ ID NO: 78939.

The probes having the nucleotide sequence represented by SEQ ID NOS: 122948, 122949, 122950, 122951, 122952, 122953, 122954, 122955, 122956, 122957, 122958 were selected from the ORFs having the nucleotide sequence represented by SEQ ID NO: 78940.

The probes having the nucleotide sequence represented by SEQ ID NOS: 122959, 122960, 122961, 122962, 122963, 122964, 122965, 122966, 122967, 122968, 122969 were selected from the ORFs having the nucleotide sequence represented by SEQ ID NO: 78941.

The probes having the nucleotide sequence represented by SEQ ID NOS: 122970, 122971, 122972, 122973, 122974, 122975, 122976, 122977, 122978, 122979, 122980 were selected from the ORFs having the nucleotide sequence represented by SEQ ID NO: 78942.

The probes having the nucleotide sequence represented by SEQ ID NOS: 122981, 122982, 122983, 122984, 122985, 122986, 122987, 122988, 122989, 122990, 122991 were selected from the ORFs having the nucleotide sequence represented by SEQ ID NO: 78943.

The probes having the nucleotide sequence represented by SEQ ID NOS: 122992, 122993, 122994, 122995, 122996, 122997, 122998, 122999, 123000, 123001, 123002 were selected from the ORFs having the nucleotide sequence represented by SEQ ID NO: 78944.

The probes having the nucleotide sequence represented by SEQ ID NOS: 123003, 123004, 123005, 123006, 123007, 123008, 123009, 123010, 123011, 123012, 123013 were selected from the ORFs having the nucleotide sequence represented by SEQ ID NO: 78945.

The probes having the nucleotide sequence represented by SEQ ID NOS: 123014, 123015, 123016, 123017, 123018, 123019, 123020, 123021, 123022, 123023, 123024 were selected from the ORFs having the nucleotide sequence represented by SEQ ID NO: 78946.

The probes having the nucleotide sequence represented by SEQ ID NOS: 123025, 123026, 123027, 123028, 123029, 123030, 123031, 123032, 123033, 123034, 123035 were selected from the ORFs having the nucleotide sequence represented by SEQ ID NO: 78947.

The probes having the nucleotide sequence represented by SEQ ID NOS: 123036, 123037, 123038, 123039, 123040, 123041, 123042, 123043, 123044, 123045, 123046 were selected from the ORFs having the nucleotide sequence represented by SEQ ID NO: 78948.

The probes having the nucleotide sequence represented by SEQ ID NOS: 123047, 123048, 123049, 123050, 123051, 123052, 123053, 123054, 123055, 123056, 123057 were selected from the ORFs having the nucleotide sequence represented by SEQ ID NO: 78949.

The probes having the nucleotide sequence represented by SEQ ID NOS: 123058, 123059, 123060, 123061, 123062, 123063, 123064, 123065, 123066, 123067, 123068 were selected from the ORFs having the nucleotide sequence represented by SEQ ID NO: 78950.

The probes having the nucleotide sequence represented by SEQ ID NOS: 123069, 123070, 123071, 123072, 123073, 123074, 123075, 123076, 123077, 123078, 123079 were selected from the ORFs having the nucleotide sequence represented by SEQ ID NO: 78951.

The probes having the nucleotide sequence represented by SEQ ID NOS: 123080, 123081, 123082, 123083, 123084, 123085, 123086, 123087, 123088, 123089, 123090 were selected from the ORFs having the nucleotide sequence represented by SEQ ID NO: 78952.

The probes having the nucleotide sequence represented by SEQ ID NOS: 123091, 123092, 123093, 123094, 123095, 123096, 123097, 123098, 123099, 123100, 123101 were selected from the ORFs having the nucleotide sequence represented by SEQ ID NO: 78953.

The probes having the nucleotide sequence represented by SEQ ID NOS: 123102, 123103, 123104, 123105, 123106, 123107, 123108, 123109, 123110, 123111, 123112 were selected from the ORFs having the nucleotide sequence represented by SEQ ID NO: 78954.

The probes having the nucleotide sequence represented by SEQ ID NOS: 123113, 123114, 123115, 123116, 123117, 123118, 123119, 123120, 123121, 123122, 123123 were selected from the ORFs having the nucleotide sequence represented by SEQ ID NO: 78955.

The probes having the nucleotide sequence represented by SEQ ID NOS: 123124, 123125, 123126, 123127, 123128, 123129, 123130, 123131, 123132, 123133, 123134 were selected from the ORFs having the nucleotide sequence represented by SEQ ID NO: 78956.

The probes having the nucleotide sequence represented by SEQ ID NOS: 123135, 123136, 123137, 123138, 123139, 123140, 123141, 123142, 123143, 123144, 123145 were selected from the ORFs having the nucleotide sequence represented by SEQ ID NO: 78957.

The probes having the nucleotide sequence represented by SEQ ID NOS: 123146, 123147, 123148, 123149, 123150, 123151, 123152, 123153, 123154, 123155, 123156 were selected from the ORFs having the nucleotide sequence represented by SEQ ID NO: 78958.

The probes having the nucleotide sequence represented by SEQ ID NOS: 123157, 123158, 123159, 123160, 123161, 123162, 123163, 123164, 123165, 123166, 123167 were selected from the ORFs having the nucleotide sequence represented by SEQ ID NO: 78959.

The probes having the nucleotide sequence represented by SEQ ID NOS: 123168, 123169, 123170, 123171, 123172, 123173, 123174, 123175, 123176, 123177, 123178 were selected from the ORFs having the nucleotide sequence represented by SEQ ID NO: 78960.

The probes having the nucleotide sequence represented by SEQ ID NOS: 123179, 123180, 123181, 123182, 123183, 123184, 123185, 123186, 123187, 123188, 123189 were selected from the ORFs having the nucleotide sequence represented by SEQ ID NO: 78961.

The probes having the nucleotide sequence represented by SEQ ID NOS: 123190, 123191, 123192, 123193, 123194, 123195, 123196, 123197, 123198, 123199, 123200 were selected from the ORFs having the nucleotide sequence represented by SEQ ID NO: 78962.

The probes having the nucleotide sequence represented by SEQ ID NOS: 123201, 123202, 123203, 123204, 123205, 123206, 123207, 123208, 123209, 123210, 123211 were selected from the ORFs having the nucleotide sequence represented by SEQ ID NO: 78963.

The probes having the nucleotide sequence represented by SEQ ID NOS: 123212, 123213, 123214, 123215, 123216, 123217, 123218, 123219, 123220, 123221, 123222 were selected from the ORFs having the nucleotide sequence represented by SEQ ID NO: 78964.

The probes having the nucleotide sequence represented by SEQ ID NOS: 123223, 123224, 123225, 123226, 123227, 123228, 123229, 123230, 123231, 123232, 123233 were selected from the ORFs having the nucleotide sequence represented by SEQ ID NO: 78965.

The probes having the nucleotide sequence represented by SEQ ID NOS: 123234, 123235, 123236, 123237, 123238, 123239, 123240, 123241, 123242, 123243, 123244 were selected from the ORFs having the nucleotide sequence represented by SEQ ID NO: 78966.

The probes having the nucleotide sequence represented by SEQ ID NOS: 123245, 123246, 123247, 123248, 123249, 123250, 123251, 123252, 123253, 123254, 123255 were selected from the ORFs having the nucleotide sequence represented by SEQ ID NO: 78967.

The probes having the nucleotide sequence represented by SEQ ID NOS: 123256, 123257, 123258, 123259, 123260, 123261, 123262, 123263, 123264, 123265, 123266 were selected from the ORFs having the nucleotide sequence represented by SEQ ID NO: 78968.

The probes having the nucleotide sequence represented by SEQ ID NOS: 123267, 123268, 123269, 123270, 123271, 123272, 123273, 123274, 123275, 123276, 123277 were selected from the ORFs having the nucleotide sequence represented by SEQ ID NO: 78969.

The probes having the nucleotide sequence represented by SEQ ID NOS: 123278, 123279, 123280, 123281, 123282, 123283, 123284, 123285, 123286, 123287, 123288 were selected from the ORFs having the nucleotide sequence represented by SEQ ID NO: 78970.

The probes having the nucleotide sequence represented by SEQ ID NOS: 123289, 123290, 123291, 123292, 123293, 123294, 123295, 123296, 123297, 123298, 123299 were selected from the ORFs having the nucleotide sequence represented by SEQ ID NO: 78971.

The probes having the nucleotide sequence represented by SEQ ID NOS: 123300, 123301, 123302, 123303, 123304, 123305, 123306, 123307, 123308, 123309, 123310 were selected from the ORFs having the nucleotide sequence represented by SEQ ID NO: 78972.

The probes having the nucleotide sequence represented by SEQ ID NOS: 123311, 123312, 123313, 123314, 123315, 123316, 123317, 123318, 123319, 123320, 123321 were selected from the ORFs having the nucleotide sequence represented by SEQ ID NO: 78973.

The probes having the nucleotide sequence represented by SEQ ID NOS: 123322, 123323, 123324, 123325, 123326, 123327, 123328, 123329, 123330, 123331, 123332 were selected from the ORFs having the nucleotide sequence represented by SEQ ID NO: 78974.

The probes having the nucleotide sequence represented by SEQ ID NOS: 123333, 123334, 123335, 123336, 123337, 123338, 123339, 123340, 123341, 123342, 123343 were selected from the ORFs having the nucleotide sequence represented by SEQ ID NO: 78975.

The probes having the nucleotide sequence represented by SEQ ID NOS: 123344, 123345, 123346, 123347, 123348, 123349, 123350, 123351, 123352, 123353, 123354 were selected from the ORFs having the nucleotide sequence represented by SEQ ID NO: 78976.

The probes having the nucleotide sequence represented by SEQ ID NOS: 123355, 123356, 123357, 123358, 123359, 123360, 123361, 123362, 123363, 123364, 123365 were selected from the ORFs having the nucleotide sequence represented by SEQ ID NO: 78977.

The probes having the nucleotide sequence represented by SEQ ID NOS: 123366, 123367, 123368, 123369, 123370, 123371, 123372, 123373, 123374, 123375, 123376, 123377, 123378, 123379, 123380, 123381, 123382, 123383, 123384, 123385, 123386, 123387 were selected from the ORFs having the nucleotide sequence represented by SEQ ID NO: 78978.

The probes having the nucleotide sequence represented by SEQ ID NOS: 123388, 123389, 123390, 123391, 123392, 123393, 123394, 123395, 123396, 123397, 123398 were selected from the ORFs having the nucleotide sequence represented by SEQ ID NO: 78979.

The probes having the nucleotide sequence represented by SEQ ID NOS: 123399, 123400, 123401, 123402, 123403, 123404, 123405, 123406, 123407, 123408, 123409 were selected from the ORFs having the nucleotide sequence represented by SEQ ID NO: 78980.

The probes having the nucleotide sequence represented by SEQ ID NOS: 123410, 123411, 123412, 123413, 123414, 123415, 123416, 123417, 123418, 123419, 123420 were selected from the ORFs having the nucleotide sequence represented by SEQ ID NO: 78981.

The probes having the nucleotide sequence represented by SEQ ID NOS: 123421, 123422, 123423, 123424, 123425, 123426, 123427, 123428, 123429, 123430, 123431 were selected from the ORFs having the nucleotide sequence represented by SEQ ID NO: 78982.

The probes having the nucleotide sequence represented by SEQ ID NOS: 123432, 123433, 123434, 123435, 123436, 123437, 123438, 123439, 123440, 123441, 123442 were selected from the ORFs having the nucleotide sequence represented by SEQ ID NO: 78983.

The probes having the nucleotide sequence represented by SEQ ID NOS: 123443, 123444, 123445, 123446, 123447, 123448, 123449, 123450, 123451, 123452, 123453 were selected from the ORFs having the nucleotide sequence represented by SEQ ID NO: 78984.

The probes having the nucleotide sequence represented by SEQ ID NOS: 123454, 123455, 123456, 123457, 123458, 123459, 123460, 123461, 123462, 123463, 123464 were selected from the ORFs having the nucleotide sequence represented by SEQ ID NO: 78985.

The probes having the nucleotide sequence represented by SEQ ID NOS: 123465, 123466, 123467, 123468, 123469, 123470, 123471, 123472, 123473, 123474, 123475 were selected from the ORFs having the nucleotide sequence represented by SEQ ID NO: 78986.

The probes having the nucleotide sequence represented by SEQ ID NOS: 123476, 123477, 123478, 123479, 123480, 123481, 123482, 123483, 123484, 123485, 123486 were selected from the ORFs having the nucleotide sequence represented by SEQ ID NO: 78987.

The probes having the nucleotide sequence represented by SEQ ID NOS: 123487, 123488, 123489, 123490, 123491, 123492, 123493, 123494, 123495, 123496, 123497 were selected from the ORFs having the nucleotide sequence represented by SEQ ID NO: 78988.

The probes having the nucleotide sequence represented by SEQ ID NOS: 123498, 123499, 123500, 123501, 123502, 123503, 123504, 123505, 123506, 123507, 123508 were selected from the ORFs having the nucleotide sequence represented by SEQ ID NO: 78989.

The probes having the nucleotide sequence represented by SEQ ID NOS: 123509, 123510, 123511, 123512, 123513, 123514, 123515, 123516, 123517, 123518, 123519 were selected from the ORFs having the nucleotide sequence represented by SEQ ID NO: 78990.

The probes having the nucleotide sequence represented by SEQ ID NOS: 123520, 123521, 123522, 123523, 123524, 123525, 123526, 123527, 123528, 123529, 123530 were selected from the ORFs having the nucleotide sequence represented by SEQ ID NO: 78991.

The probes having the nucleotide sequence represented by SEQ ID NOS: 123531, 123532, 123533, 123534, 123535, 123536, 123537, 123538, 123539, 123540, 123541 were selected from the ORFs having the nucleotide sequence represented by SEQ ID NO: 78992.

The probes having the nucleotide sequence represented by SEQ ID NOS: 123542, 123543, 123544, 123545, 123546, 123547, 123548, 123549, 123550, 123551, 123552 were selected from the ORFs having the nucleotide sequence represented by SEQ ID NO: 78993.

The probes having the nucleotide sequence represented by SEQ ID NOS: 123553, 123554, 123555, 123556, 123557, 123558, 123559, 123560, 123561, 123562, 123563 were selected from the ORFs having the nucleotide sequence represented by SEQ ID NO: 78994.

The probes having the nucleotide sequence represented by SEQ ID NOS: 123564, 123565, 123566, 123567, 123568, 123569, 123570, 123571, 123572, 123573, 123574 were selected from the ORFs having the nucleotide sequence represented by SEQ ID NO: 78995.

The probes having the nucleotide sequence represented by SEQ ID NOS: 123575, 123576, 123577, 123578, 123579, 123580, 123581, 123582, 123583, 123584, 123585 were selected from the ORFs having the nucleotide sequence represented by SEQ ID NO: 78996.

The probes having the nucleotide sequence represented by SEQ ID NOS: 123586, 123587, 123588, 123589, 123590, 123591, 123592, 123593, 123594, 123595, 123596 were selected from the ORFs having the nucleotide sequence represented by SEQ ID NO: 78997.

The probes having the nucleotide sequence represented by SEQ ID NOS: 123597, 123598, 123599, 123600, 123601, 123602, 123603, 123604, 123605, 123606, 123607 were selected from the ORFs having the nucleotide sequence represented by SEQ ID NO: 78998.

The probes having the nucleotide sequence represented by SEQ ID NOS: 123608, 123609, 123610, 123611, 123612, 123613, 123614, 123615, 123616, 123617, 123618 were selected from the ORFs having the nucleotide sequence represented by SEQ ID NO: 78999.

The probes having the nucleotide sequence represented by SEQ ID NOS: 123619, 123620, 123621, 123622, 123623, 123624, 123625, 123626, 123627, 123628, 123629 were selected from the ORFs having the nucleotide sequence represented by SEQ ID NO: 79000.

The probes having the nucleotide sequence represented by SEQ ID NOS: 123630, 123631, 123632, 123633, 123634, 123635, 123636, 123637, 123638, 123639, 123640 were selected from the ORFs having the nucleotide sequence represented by SEQ ID NO: 79001.

The probes having the nucleotide sequence represented by SEQ ID NOS: 123641, 123642, 123643, 123644, 123645, 123646, 123647, 123648, 123649, 123650, 123651 were selected from the ORFs having the nucleotide sequence represented by SEQ ID NO: 79002.

The probes having the nucleotide sequence represented by SEQ ID NOS: 123652, 123653, 123654, 123655, 123656, 123657, 123658, 123659, 123660, 123661, 123662 were selected from the ORFs having the nucleotide sequence represented by SEQ ID NO: 79003.

The probes having the nucleotide sequence represented by SEQ ID NOS: 123663, 123664, 123665, 123666, 123667, 123668, 123669, 123670, 123671, 123672, 123673 were selected from the ORFs having the nucleotide sequence represented by SEQ ID NO: 79004.

The probes having the nucleotide sequence represented by SEQ ID NOS: 123674, 123675, 123676, 123677, 123678, 123679, 123680, 123681, 123682, 123683, 123684 were selected from the ORFs having the nucleotide sequence represented by SEQ ID NO: 79005.

The probes having the nucleotide sequence represented by SEQ ID NOS: 123685, 123686, 123687, 123688, 123689, 123690, 123691, 123692, 123693, 123694, 123695 were selected from the ORFs having the nucleotide sequence represented by SEQ ID NO: 79006.

The probes having the nucleotide sequence represented by SEQ ID NOS: 123696, 123697, 123698, 123699, 123700, 123701, 123702, 123703, 123704, 123705, 123706 were selected from the ORFs having the nucleotide sequence represented by SEQ ID NO: 79007.

The probes having the nucleotide sequence represented by SEQ ID NOS: 123707, 123708, 123709, 123710, 123711, 123712, 123713, 123714, 123715, 123716, 123717 were selected from the ORFs having the nucleotide sequence represented by SEQ ID NO: 79008.

The probes having the nucleotide sequence represented by SEQ ID NOS: 123718, 123719, 123720, 123721, 123722, 123723, 123724, 123725, 123726, 123727, 123728 were selected from the ORFs having the nucleotide sequence represented by SEQ ID NO: 79009.

The probes having the nucleotide sequence represented by SEQ ID NOS: 123729, 123730, 123731, 123732, 123733, 123734, 123735, 123736, 123737, 123738, 123739 were selected from the ORFs having the nucleotide sequence represented by SEQ ID NO: 79010.

The probes having the nucleotide sequence represented by SEQ ID NOS: 123740, 123741, 123742, 123743, 123744, 123745, 123746, 123747, 123748, 123749, 123750 were selected from the ORFs having the nucleotide sequence represented by SEQ ID NO: 79011.

The probes having the nucleotide sequence represented by SEQ ID NOS: 123751, 123752, 123753, 123754, 123755, 123756, 123757, 123758, 123759, 123760, 123761 were selected from the ORFs having the nucleotide sequence represented by SEQ ID NO: 79012.

The probes having the nucleotide sequence represented by SEQ ID NOS: 123762, 123763, 123764, 123765, 123766, 123767, 123768, 123769, 123770, 123771, 123772 were selected from the ORFs having the nucleotide sequence represented by SEQ ID NO: 79013.

The probes having the nucleotide sequence represented by SEQ ID NOS: 123773, 123774, 123775, 123776, 123777, 123778, 123779, 123780, 123781, 123782, 123783 were selected from the ORFs having the nucleotide sequence represented by SEQ ID NO: 79014.

The probes having the nucleotide sequence represented by SEQ ID NOS: 123784, 123785, 123786, 123787, 123788, 123789, 123790, 123791, 123792, 123793, 123794 were selected from the ORFs having the nucleotide sequence represented by SEQ ID NO: 79015.

The probes having the nucleotide sequence represented by SEQ ID NOS: 123795, 123796, 123797, 123798, 123799, 123800, 123801, 123802, 123803, 123804, 123805 were selected from the ORFs having the nucleotide sequence represented by SEQ ID NO: 79016.

The probes having the nucleotide sequence represented by SEQ ID NOS: 123806, 123807, 123808, 123809, 123810, 123811, 123812, 123813, 123814, 123815, 123816 were selected from the ORFs having the nucleotide sequence represented by SEQ ID NO: 79017.

The probes having the nucleotide sequence represented by SEQ ID NOS: 123817, 123818, 123819, 123820, 123821, 123822, 123823, 123824, 123825, 123826, 123827 were selected from the ORFs having the nucleotide sequence represented by SEQ ID NO: 79018.

The probes having the nucleotide sequence represented by SEQ ID NOS: 123828, 123829, 123830, 123831, 123832, 123833, 123834, 123835, 123836, 123837, 123838 were selected from the ORFs having the nucleotide sequence represented by SEQ ID NO: 79019.

The probes having the nucleotide sequence represented by SEQ ID NOS: 123839, 123840, 123841, 123842, 123843, 123844, 123845, 123846, 123847, 123848, 123849 were selected from the ORFs having the nucleotide sequence represented by SEQ ID NO: 79020.

The probes having the nucleotide sequence represented by SEQ ID NOS: 123850, 123851, 123852, 123853, 123854, 123855, 123856, 123857, 123858, 123859, 123860 were selected from the ORFs having the nucleotide sequence represented by SEQ ID NO: 79021.

The probes having the nucleotide sequence represented by SEQ ID NOS: 123861, 123862, 123863, 123864, 123865, 123866, 123867, 123868, 123869, 123870, 123871 were selected from the ORFs having the nucleotide sequence represented by SEQ ID NO: 79022.

The probes having the nucleotide sequence represented by SEQ ID NOS: 123872, 123873, 123874, 123875, 123876, 123877, 123878, 123879, 123880, 123881, 123882 were selected from the ORFs having the nucleotide sequence represented by SEQ ID NO: 79023.

The probes having the nucleotide sequence represented by SEQ ID NOS: 123883, 123884, 123885, 123886, 123887, 123888, 123889, 123890, 123891, 123892, 123893 were selected from the ORFs having the nucleotide sequence represented by SEQ ID NO: 79024.

The probes having the nucleotide sequence represented by SEQ ID NOS: 123894, 123895, 123896, 123897, 123898, 123899, 123900, 123901, 123902, 123903, 123904 were selected from the ORFs having the nucleotide sequence represented by SEQ ID NO: 79025.

The probes having the nucleotide sequence represented by SEQ ID NOS: 123905, 123906, 123907, 123908, 123909, 123910, 123911, 123912, 123913, 123914, 123915 were selected from the ORFs having the nucleotide sequence represented by SEQ ID NO: 79026.

The probes having the nucleotide sequence represented by SEQ ID NOS: 123916, 123917, 123918, 123919, 123920, 123921, 123922, 123923, 123924, 123925, 123926 were selected from the ORFs having the nucleotide sequence represented by SEQ ID NO: 79027.

The probes having the nucleotide sequence represented by SEQ ID NOS: 123927, 123928, 123929, 123930, 123931, 123932, 123933, 123934, 123935, 123936, 123937 were selected from the ORFs having the nucleotide sequence represented by SEQ ID NO: 79028.

The probes having the nucleotide sequence represented by SEQ ID NOS: 123938, 123939, 123940, 123941, 123942, 123943, 123944, 123945, 123946, 123947, 123948 were selected from the ORFs having the nucleotide sequence represented by SEQ ID NO: 79029.

The probes having the nucleotide sequence represented by SEQ ID NOS: 123949, 123950, 123951, 123952, 123953, 123954, 123955, 123956, 123957, 123958, 123959 were selected from the ORFs having the nucleotide sequence represented by SEQ ID NO: 79030.

The probes having the nucleotide sequence represented by SEQ ID NOS: 123960, 123961, 123962, 123963, 123964, 123965, 123966, 123967, 123968, 123969, 123970 were selected from the ORFs having the nucleotide sequence represented by SEQ ID NO: 79031.

The probes having the nucleotide sequence represented by SEQ ID NOS: 123971, 123972, 123973, 123974, 123975, 123976, 123977, 123978, 123979, 123980, 123981 were selected from the ORFs having the nucleotide sequence represented by SEQ ID NO: 79032.

The probes having the nucleotide sequence represented by SEQ ID NOS: 123982, 123983, 123984, 123985, 123986, 123987, 123988, 123989, 123990, 123991, 123992 were selected from the ORFs having the nucleotide sequence represented by SEQ ID NO: 79033.

The probes having the nucleotide sequence represented by SEQ ID NOS: 123993, 123994, 123995, 123996, 123997, 123998, 123999, 124000, 124001, 124002, 124003 were selected from the ORFs having the nucleotide sequence represented by SEQ ID NO: 79034.

The probes having the nucleotide sequence represented by SEQ ID NOS: 124004, 124005, 124006, 124007, 124008, 124009, 124010, 124011, 124012, 124013, 124014 were selected from the ORFs having the nucleotide sequence represented by SEQ ID NO: 79035.

The probes having the nucleotide sequence represented by SEQ ID NOS: 124015, 124016, 124017, 124018, 124019, 124020, 124021, 124022, 124023, 124024, 124025 were selected from the ORFs having the nucleotide sequence represented by SEQ ID NO: 79036.

The probes having the nucleotide sequence represented by SEQ ID NOS: 124026, 124027, 124028, 124029, 124030, 124031, 124032, 124033, 124034, 124035, 124036 were selected from the ORFs having the nucleotide sequence represented by SEQ ID NO: 79037.

The probes having the nucleotide sequence represented by SEQ ID NOS: 124037, 124038, 124039, 124040, 124041, 124042, 124043, 124044, 124045, 124046, 124047 were selected from the ORFs having the nucleotide sequence represented by SEQ ID NO: 79038.

The probes having the nucleotide sequence represented by SEQ ID NOS: 124048, 124049, 124050, 124051, 124052, 124053, 124054, 124055, 124056, 124057, 124058 were selected from the ORFs having the nucleotide sequence represented by SEQ ID NO: 79039.

The probes having the nucleotide sequence represented by SEQ ID NOS: 124059, 124060, 124061, 124062, 124063, 124064, 124065, 124066, 124067, 124068, 124069 were selected from the ORFs having the nucleotide sequence represented by SEQ ID NO: 79040.

The probes having the nucleotide sequence represented by SEQ ID NOS: 124070, 124071, 124072, 124073, 124074, 124075, 124076, 124077, 124078, 124079, 124080 were selected from the ORFs having the nucleotide sequence represented by SEQ ID NO: 79041.

The probes having the nucleotide sequence represented by SEQ ID NOS: 124081, 124082, 124083, 124084, 124085, 124086, 124087, 124088, 124089, 124090, 124091 were selected from the ORFs having the nucleotide sequence represented by SEQ ID NO: 79042.

The probes having the nucleotide sequence represented by SEQ ID NOS: 124092, 124093, 124094, 124095, 124096, 124097, 124098, 124099, 124100, 124101, 124102 were selected from the ORFs having the nucleotide sequence represented by SEQ ID NO: 79043.

The probes having the nucleotide sequence represented by SEQ ID NOS: 124103, 124104, 124105, 124106, 124107, 124108, 124109, 124110, 124111, 124112, 124113 were selected from the ORFs having the nucleotide sequence represented by SEQ ID NO: 79044.

The probes having the nucleotide sequence represented by SEQ ID NOS: 124114, 124115, 124116, 124117, 124118, 124119, 124120, 124121, 124122, 124123, 124124 were selected from the ORFs having the nucleotide sequence represented by SEQ ID NO: 79045.

The probes having the nucleotide sequence represented by SEQ ID NOS: 124125, 124126, 124127, 124128, 124129, 124130, 124131, 124132, 124133, 124134, 124135 were selected from the ORFs having the nucleotide sequence represented by SEQ ID NO: 79046.

The probes having the nucleotide sequence represented by SEQ ID NOS: 124136, 124137, 124138, 124139, 124140, 124141, 124142, 124143, 124144, 124145, 124146 were selected from the ORFs having the nucleotide sequence represented by SEQ ID NO: 79047.

The probes having the nucleotide sequence represented by SEQ ID NOS: 124147, 124148, 124149, 124150, 124151, 124152, 124153, 124154, 124155, 124156, 124157 were selected from the ORFs having the nucleotide sequence represented by SEQ ID NO: 79048.

The probes having the nucleotide sequence represented by SEQ ID NOS: 124158, 124159, 124160, 124161, 124162, 124163, 124164, 124165, 124166, 124167, 124168 were selected from the ORFs having the nucleotide sequence represented by SEQ ID NO: 79049.

The probes having the nucleotide sequence represented by SEQ ID NOS: 124169, 124170, 124171, 124172, 124173, 124174, 124175, 124176, 124177, 124178, 124179 were selected from the ORFs having the nucleotide sequence represented by SEQ ID NO: 79050.

The probes having the nucleotide sequence represented by SEQ ID NOS: 124180, 124181, 124182, 124183, 124184, 124185, 124186, 124187, 124188, 124189, 124190 were selected from the ORFs having the nucleotide sequence represented by SEQ ID NO: 79051.

The probes having the nucleotide sequence represented by SEQ ID NOS: 124191, 124192, 124193, 124194, 124195, 124196, 124197, 124198, 124199, 124200, 124201, 124202, 124203, 124204, 124205, 124206, 124207, 124208, 124209, 124210, 124211, 124212 were selected from the ORFs having the nucleotide sequence represented by SEQ ID NO: 79052.

The probes having the nucleotide sequence represented by SEQ ID NOS: 124213, 124214, 124215, 124216, 124217, 124218, 124219, 124220, 124221, 124222, 124223 were selected from the ORFs having the nucleotide sequence represented by SEQ ID NO: 79053.

The probes having the nucleotide sequence represented by SEQ ID NOS: 124224, 124225, 124226, 124227, 124228, 124229, 124230, 124231, 124232, 124233, 124234 were selected from the ORFs having the nucleotide sequence represented by SEQ ID NO: 79054.

The probes having the nucleotide sequence represented by SEQ ID NOS: 124235, 124236, 124237, 124238, 124239, 124240, 124241, 124242, 124243, 124244, 124245 were selected from the ORFs having the nucleotide sequence represented by SEQ ID NO: 79055.

The probes having the nucleotide sequence represented by SEQ ID NOS: 124246, 124247, 124248, 124249, 124250, 124251, 124252, 124253, 124254, 124255, 124256 were selected from the ORFs having the nucleotide sequence represented by SEQ ID NO: 79056.

The probes having the nucleotide sequence represented by SEQ ID NOS: 124257, 124258, 124259, 124260, 124261, 124262, 124263, 124264, 124265, 124266, 124267 were selected from the ORFs having the nucleotide sequence represented by SEQ ID NO: 79057.

The probes having the nucleotide sequence represented by SEQ ID NOS: 124268, 124269, 124270, 124271, 124272, 124273, 124274, 124275, 124276, 124277, 124278 were selected from the ORFs having the nucleotide sequence represented by SEQ ID NO: 79058.

The probes having the nucleotide sequence represented by SEQ ID NOS: 124279, 124280, 124281, 124282, 124283, 124284, 124285, 124286, 124287, 124288, 124289 were selected from the ORFs having the nucleotide sequence represented by SEQ ID NO: 79059.

The probes having the nucleotide sequence represented by SEQ ID NOS: 124290, 124291, 124292, 124293, 124294, 124295, 124296, 124297, 124298, 124299, 124300 were selected from the ORFs having the nucleotide sequence represented by SEQ ID NO: 79060.

The probes having the nucleotide sequence represented by SEQ ID NOS: 124301, 124302, 124303, 124304, 124305, 124306, 124307, 124308, 124309, 124310, 124311 were selected from the ORFs having the nucleotide sequence represented by SEQ ID NO: 79061.

The probes having the nucleotide sequence represented by SEQ ID NOS: 124312, 124313, 124314, 124315, 124316, 124317, 124318, 124319, 124320, 124321, 124322 were selected from the ORFs having the nucleotide sequence represented by SEQ ID NO: 79062.

The probes having the nucleotide sequence represented by SEQ ID NOS: 124323, 124324, 124325, 124326, 124327, 124328, 124329, 124330, 124331, 124332, 124333 were selected from the ORFs having the nucleotide sequence represented by SEQ ID NO: 79063.

The probes having the nucleotide sequence represented by SEQ ID NOS: 124334, 124335, 124336, 124337, 124338, 124339, 124340, 124341, 124342, 124343, 124344 were selected from the ORFs having the nucleotide sequence represented by SEQ ID NO: 79064.

The probes having the nucleotide sequence represented by SEQ ID NOS: 124345, 124346, 124347, 124348, 124349, 124350, 124351, 124352, 124353, 124354, 124355 were selected from the ORFs having the nucleotide sequence represented by SEQ ID NO: 79065.

The probes having the nucleotide sequence represented by SEQ ID NOS: 124356, 124357, 124358, 124359, 124360, 124361, 124362, 124363, 124364, 124365, 124366 were selected from the ORFs having the nucleotide sequence represented by SEQ ID NO: 79066.

The probes having the nucleotide sequence represented by SEQ ID NOS: 124367, 124368, 124369, 124370, 124371, 124372, 124373, 124374, 124375, 124376, 124377 were selected from the ORFs having the nucleotide sequence represented by SEQ ID NO: 79067.

The probes having the nucleotide sequence represented by SEQ ID NOS: 124378, 124379, 124380, 124381, 124382, 124383, 124384, 124385, 124386, 124387, 124388 were selected from the ORFs having the nucleotide sequence represented by SEQ ID NO: 79068.

The probes having the nucleotide sequence represented by SEQ ID NOS: 124389, 124390, 124391, 124392, 124393, 124394, 124395, 124396, 124397, 124398, 124399 were selected from the ORFs having the nucleotide sequence represented by SEQ ID NO: 79069.

The probes having the nucleotide sequence represented by SEQ ID NOS: 124400, 124401, 124402, 124403, 124404, 124405, 124406, 124407, 124408, 124409, 124410 were selected from the ORFs having the nucleotide sequence represented by SEQ ID NO: 79070.

The probes having the nucleotide sequence represented by SEQ ID NOS: 124411, 124412, 124413, 124414, 124415, 124416, 124417, 124418, 124419, 124420, 124421 were selected from the ORFs having the nucleotide sequence represented by SEQ ID NO: 79071.

The probes having the nucleotide sequence represented by SEQ ID NOS: 124422, 124423, 124424, 124425, 124426, 124427, 124428, 124429, 124430, 124431, 124432 were selected from the ORFs having the nucleotide sequence represented by SEQ ID NO: 79072.

The probes having the nucleotide sequence represented by SEQ ID NOS: 124433, 124434, 124435, 124436, 124437, 124438, 124439, 124440, 124441, 124442, 124443 were selected from the ORFs having the nucleotide sequence represented by SEQ ID NO: 79073.

The probes having the nucleotide sequence represented by SEQ ID NOS: 124444, 124445, 124446, 124447, 124448, 124449, 124450, 124451, 124452, 124453, 124454 were selected from the ORFs having the nucleotide sequence represented by SEQ ID NO: 79074.

The probes having the nucleotide sequence represented by SEQ ID NOS: 124455, 124456, 124457, 124458, 124459, 124460, 124461, 124462, 124463, 124464, 124465 were selected from the ORFs having the nucleotide sequence represented by SEQ ID NO: 79075.

The probes having the nucleotide sequence represented by SEQ ID NOS: 124466, 124467, 124468, 124469, 124470, 124471, 124472, 124473, 124474, 124475, 124476 were selected from the ORFs having the nucleotide sequence represented by SEQ ID NO: 79076.

The probes having the nucleotide sequence represented by SEQ ID NOS: 124477, 124478, 124479, 124480, 124481, 124482, 124483, 124484, 124485, 124486, 124487 were selected from the ORFs having the nucleotide sequence represented by SEQ ID NO: 79077.

The probes having the nucleotide sequence represented by SEQ ID NOS: 124488, 124489, 124490, 124491, 124492, 124493, 124494, 124495, 124496, 124497, 124498 were selected from the ORFs having the nucleotide sequence represented by SEQ ID NO: 79078.

The probes having the nucleotide sequence represented by SEQ ID NOS: 124499, 124500, 124501, 124502, 124503, 124504, 124505, 124506, 124507, 124508, 124509 were selected from the ORFs having the nucleotide sequence represented by SEQ ID NO: 79079.

The probes having the nucleotide sequence represented by SEQ ID NOS: 124510, 124511, 124512, 124513, 124514, 124515, 124516, 124517, 124518, 124519, 124520 were selected from the ORFs having the nucleotide sequence represented by SEQ ID NO: 79080.

The probes having the nucleotide sequence represented by SEQ ID NOS: 124521, 124522, 124523, 124524, 124525, 124526, 124527, 124528, 124529, 124530, 124531 were selected from the ORFs having the nucleotide sequence represented by SEQ ID NO: 79081.

The probes having the nucleotide sequence represented by SEQ ID NOS: 124532, 124533, 124534, 124535, 124536, 124537, 124538, 124539, 124540, 124541, 124542 were selected from the ORFs having the nucleotide sequence represented by SEQ ID NO: 79082.

The probes having the nucleotide sequence represented by SEQ ID NOS: 124543, 124544, 124545, 124546, 124547, 124548, 124549, 124550, 124551, 124552, 124553 were selected from the ORFs having the nucleotide sequence represented by SEQ ID NO: 79083.

The probes having the nucleotide sequence represented by SEQ ID NOS: 124554, 124555, 124556, 124557, 124558, 124559, 124560, 124561, 124562, 124563, 124564 were selected from the ORFs having the nucleotide sequence represented by SEQ ID NO: 79084.

The probes having the nucleotide sequence represented by SEQ ID NOS: 124565, 124566, 124567, 124568, 124569, 124570, 124571, 124572, 124573, 124574, 124575 were selected from the ORFs having the nucleotide sequence represented by SEQ ID NO: 79085.

The probes having the nucleotide sequence represented by SEQ ID NOS: 124576, 124577, 124578, 124579, 124580, 124581, 124582, 124583, 124584, 124585, 124586 were selected from the ORFs having the nucleotide sequence represented by SEQ ID NO: 79086.

The probes having the nucleotide sequence represented by SEQ ID NOS: 124587, 124588, 124589, 124590, 124591, 124592, 124593, 124594, 124595, 124596, 124597 were selected from the ORFs having the nucleotide sequence represented by SEQ ID NO: 79087.

The probes having the nucleotide sequence represented by SEQ ID NOS: 124598, 124599, 124600, 124601, 124602, 124603, 124604, 124605, 124606, 124607, 124608 were selected from the ORFs having the nucleotide sequence represented by SEQ ID NO: 79088.

The probes having the nucleotide sequence represented by SEQ ID NOS: 124609, 124610, 124611, 124612, 124613, 124614, 124615, 124616, 124617, 124618, 124619 were selected from the ORFs having the nucleotide sequence represented by SEQ ID NO: 79089.

The probes having the nucleotide sequence represented by SEQ ID NOS: 124620, 124621, 124622, 124623, 124624, 124625, 124626, 124627, 124628, 124629, 124630 were selected from the ORFs having the nucleotide sequence represented by SEQ ID NO: 79090.

The probes having the nucleotide sequence represented by SEQ ID NOS: 124631, 124632, 124633, 124634, 124635, 124636, 124637, 124638, 124639, 124640, 124641 were selected from the ORFs having the nucleotide sequence represented by SEQ ID NO: 79091.

The probes having the nucleotide sequence represented by SEQ ID NOS: 124642, 124643, 124644, 124645, 124646, 124647, 124648, 124649, 124650, 124651, 124652 were selected from the ORFs having the nucleotide sequence represented by SEQ ID NO: 79092.

The probes having the nucleotide sequence represented by SEQ ID NOS: 124653, 124654, 124655, 124656, 124657, 124658, 124659, 124660, 124661, 124662, 124663 were selected from the ORFs having the nucleotide sequence represented by SEQ ID NO: 79093.

The probes having the nucleotide sequence represented by SEQ ID NOS: 124664, 124665, 124666, 124667, 124668, 124669, 124670, 124671, 124672, 124673, 124674 were selected from the ORFs having the nucleotide sequence represented by SEQ ID NO: 79094.

The probes having the nucleotide sequence represented by SEQ ID NOS: 124675, 124676, 124677, 124678, 124679, 124680, 124681, 124682, 124683, 124684, 124685 were selected from the ORFs having the nucleotide sequence represented by SEQ ID NO: 79095.

The probes having the nucleotide sequence represented by SEQ ID NOS: 124686, 124687, 124688, 124689, 124690, 124691, 124692, 124693, 124694, 124695, 124696 were selected from the ORFs having the nucleotide sequence represented by SEQ ID NO: 79096.

The probes having the nucleotide sequence represented by SEQ ID NOS: 124697, 124698, 124699, 124700, 124701, 124702, 124703, 124704, 124705, 124706, 124707 were selected from the ORFs having the nucleotide sequence represented by SEQ ID NO: 79097.

The probes having the nucleotide sequence represented by SEQ ID NOS: 124708, 124709, 124710, 124711, 124712, 124713, 124714, 124715, 124716, 124717, 124718 were selected from the ORFs having the nucleotide sequence represented by SEQ ID NO: 79098.

The probes having the nucleotide sequence represented by SEQ ID NOS: 124719, 124720, 124721, 124722, 124723, 124724, 124725, 124726, 124727, 124728, 124729 were selected from the ORFs having the nucleotide sequence represented by SEQ ID NO: 79099.

The probes having the nucleotide sequence represented by SEQ ID NOS: 124730, 124731, 124732, 124733, 124734, 124735, 124736, 124737, 124738, 124739, 124740 were selected from the ORFs having the nucleotide sequence represented by SEQ ID NO: 79100.

The probes having the nucleotide sequence represented by SEQ ID NOS: 124741, 124742, 124743, 124744, 124745, 124746, 124747, 124748, 124749, 124750, 124751 were selected from the ORFs having the nucleotide sequence represented by SEQ ID NO: 79101.

The probes having the nucleotide sequence represented by SEQ ID NOS: 124752, 124753, 124754, 124755, 124756, 124757, 124758, 124759, 124760, 124761, 124762 were selected from the ORFs having the nucleotide sequence represented by SEQ ID NO: 79102.

The probes having the nucleotide sequence represented by SEQ ID NOS: 124763, 124764, 124765, 124766, 124767, 124768, 124769, 124770, 124771, 124772, 124773 were selected from the ORFs having the nucleotide sequence represented by SEQ ID NO: 79103.

The probes having the nucleotide sequence represented by SEQ ID NOS: 124774, 124775, 124776, 124777, 124778, 124779, 124780, 124781, 124782, 124783, 124784 were selected from the ORFs having the nucleotide sequence represented by SEQ ID NO: 79104.

The probes having the nucleotide sequence represented by SEQ ID NOS: 124785, 124786, 124787, 124788, 124789, 124790, 124791, 124792, 124793, 124794, 124795 were selected from the ORFs having the nucleotide sequence represented by SEQ. ID NO: 79105.

The probes having the nucleotide sequence represented by SEQ ID NOS: 124796, 124797, 124798, 124799, 124800, 124801, 124802, 124803, 124804, 124805, 124806 were selected from the ORFs having the nucleotide sequence represented by SEQ ID NO: 79106.

The probes having the nucleotide sequence represented by SEQ ID NOS: 124807, 124808, 124809, 124810, 124811, 124812, 124813, 124814, 124815, 124816, 124817 were selected from the ORFs having the nucleotide sequence represented by SEQ ID NO: 79107.

The probes having the nucleotide sequence represented by SEQ ID NOS: 124818, 124819, 124820, 124821, 124822, 124823, 124824, 124825, 124826, 124827, 124828 were selected from the ORFs having the nucleotide sequence represented by SEQ ID NO: 79108.

The probes having the nucleotide sequence represented by SEQ ID NOS: 124829, 124830, 124831, 124832, 124833, 124834, 124835, 124836, 124837, 124838, 124839 were selected from the ORFs having the nucleotide sequence represented by SEQ ID NO: 79109.

The probes having the nucleotide sequence represented by SEQ ID NOS: 124840, 124841, 124842, 124843, 124844, 124845, 124846, 124847, 124848, 124849, 124850 were selected from the ORFs having the nucleotide sequence represented by SEQ ID NO: 79110.

The probes having the nucleotide sequence represented by SEQ ID NOS: 124851, 124852, 124853, 124854, 124855, 124856, 124857, 124858, 124859, 124860, 124861 were selected from the ORFs having the nucleotide sequence represented by SEQ ID NO: 79111.

The probes having the nucleotide sequence represented by SEQ ID NOS: 124862, 124863, 124864, 124865, 124866, 124867, 124868, 124869, 124870, 124871, 124872 were selected from the ORFs having the nucleotide sequence represented by SEQ ID NO: 79112.

The probes having the nucleotide sequence represented by SEQ ID NOS: 124873, 124874, 124875, 124876, 124877, 124878, 124879, 124880, 124881, 124882, 124883 were selected from the ORFs having the nucleotide sequence represented by SEQ ID NO: 79113.

The probes having the nucleotide sequence represented by SEQ ID NOS: 124884, 124885, 124886, 124887, 124888, 124889, 124890, 124891, 124892, 124893, 124894 were selected from the ORFs having the nucleotide sequence represented by SEQ ID NO: 79114.

The probes having the nucleotide sequence represented by SEQ ID NOS: 124895, 124896, 124897, 124898, 124899, 124900, 124901, 124902, 124903, 124904, 124905 were selected from the ORFs having the nucleotide sequence represented by SEQ ID NO: 79115.

The probes having the nucleotide sequence represented by SEQ ID NOS: 124906, 124907, 124908, 124909, 124910, 124911, 124912, 124913, 124914, 124915, 124916 were selected from the ORFs having the nucleotide sequence represented by SEQ ID NO: 79116.

The probes having the nucleotide sequence represented by SEQ ID NOS: 124917, 124918, 124919, 124920, 124921, 124922, 124923, 124924, 124925, 124926, 124927 were selected from the ORFs having the nucleotide sequence represented by SEQ ID NO: 79117.

The probes having the nucleotide sequence represented by SEQ ID NOS: 124928, 124929, 124930, 124931, 124932, 124933, 124934, 124935, 124936, 124937, 124938 were selected from the ORFs having the nucleotide sequence represented by SEQ ID NO: 79118.

The probes having the nucleotide sequence represented by SEQ ID NOS: 124939, 124940, 124941, 124942, 124943, 124944, 124945, 124946, 124947, 124948, 124949 were selected from the ORFs having the nucleotide sequence represented by SEQ ID NO: 79119.

The probes having the nucleotide sequence represented by SEQ ID NOS: 124950, 124951, 124952, 124953, 124954, 124955, 124956, 124957, 124958, 124959, 124960 were selected from the ORFs having the nucleotide sequence represented by SEQ ID NO: 79120.

The probes having the nucleotide sequence represented by SEQ ID NOS: 124961, 124962, 124963, 124964, 124965, 124966, 124967, 124968, 124969, 124970, 124971 were selected from the ORFs having the nucleotide sequence represented by SEQ ID NO: 79121.

The probes having the nucleotide sequence represented by SEQ ID NOS: 124972, 124973, 124974, 124975, 124976, 124977, 124978, 124979, 124980, 124981, 124982 were selected from the ORFs having the nucleotide sequence represented by SEQ ID NO: 79122.

The probes having the nucleotide sequence represented by SEQ ID NOS: 124983, 124984, 124985, 124986, 124987, 124988, 124989, 124990, 124991, 124992, 124993 were selected from the ORFs having the nucleotide sequence represented by SEQ ID NO: 79123.

The probes having the nucleotide sequence represented by SEQ ID NOS: 124994, 124995, 124996, 124997, 124998, 124999, 125000, 125001, 125002, 125003, 125004 were selected from the ORFs having the nucleotide sequence represented by SEQ ID NO: 79124.

The probes having the nucleotide sequence represented by SEQ ID NOS: 125005, 125006, 125007, 125008, 125009, 125010, 125011, 125012, 125013, 125014, 125015 were selected from the ORFs having the nucleotide sequence represented by SEQ ID NO: 79125.

The probes having the nucleotide sequence represented by SEQ ID NOS: 125016, 125017, 125018, 125019, 125020, 125021, 125022, 125023, 125024, 125025, 125026 were selected from the ORFs having the nucleotide sequence represented by SEQ ID NO: 79126.

The probes having the nucleotide sequence represented by SEQ ID NOS: 125027, 125028, 125029, 125030, 125031, 125032, 125033, 125034, 125035, 125036, 125037, 125038, 125039, 125040, 125041, 125042, 125043, 125044, 125045, 125046, 125047, 125048 were selected from the ORFs having the nucleotide sequence represented by SEQ ID NO: 79127.

The probes having the nucleotide sequence represented by SEQ ID NOS: 125049, 125050, 125051, 125052, 125053, 125054, 125055, 125056, 125057, 125058 were selected from the ORFs having the nucleotide sequence represented by SEQ ID NO: 79128.

The probes having the nucleotide sequence represented by SEQ ID NOS: 125059, 125060, 125061, 125062, 125063, 125064, 125065, 125066, 125067, 125068, 125069 were selected from the ORFs having the nucleotide sequence represented by SEQ ID NO: 79129.

The probes having the nucleotide sequence represented by SEQ ID NOS: 125070, 125071, 125072, 125073, 125074, 125075, 125076, 125077, 125078, 125079, 125080 were selected from the ORFs having the nucleotide sequence represented by SEQ ID NO: 79130.

The probes having the nucleotide sequence represented by SEQ ID NOS: 125081, 125082, 125083, 125084, 125085, 125086, 125087, 125088, 125089, 125090, 125091 were selected from the ORFs having the nucleotide sequence represented by SEQ ID NO: 79131.

The probes having the nucleotide sequence represented by SEQ ID NOS: 125092, 125093, 125094, 125095, 125096, 125097, 125098, 125099, 125100, 125101, 125102 were selected from the ORFs having the nucleotide sequence represented by SEQ ID NO: 79132.

The probes having the nucleotide sequence represented by SEQ ID NOS: 125103, 125104, 125105, 125106, 125107, 125108, 125109, 125110, 125111, 125112, 125113 were selected from the ORFs having the nucleotide sequence represented by SEQ ID NO: 79133.

The probes having the nucleotide sequence represented by SEQ ID NOS: 125114, 125115, 125116, 125117, 125118, 125119, 125120, 125121, 125122, 125123, 125124 were selected from the ORFs having the nucleotide sequence represented by SEQ ID NO: 79134.

The probes having the nucleotide sequence represented by SEQ ID NOS: 125125, 125126, 125127, 125128, 125129, 125130, 125131, 125132, 125133, 125134, 125135 were selected from the ORFs having the nucleotide sequence represented by SEQ ID NO: 79135.

The probes having the nucleotide sequence represented by SEQ ID NOS: 125136, 125137, 125138, 125139, 125140, 125141, 125142, 125143, 125144, 125145, 125146 were selected from the ORFs having the nucleotide sequence represented by SEQ ID NO: 79136.

The probes having the nucleotide sequence represented by SEQ ID NOS: 125147, 125148, 125149, 125150, 125151, 125152, 125153, 125154, 125155, 125156, 125157 were selected from the ORFs having the nucleotide sequence represented by SEQ ID NO: 79137.

The probes having the nucleotide sequence represented by SEQ ID NOS: 125158, 125159, 125160, 125161, 125162, 125163, 125164, 125165, 125166, 125167, 125168 were selected from the ORFs having the nucleotide sequence represented by SEQ ID NO: 79138.

The probes having the nucleotide sequence represented by SEQ ID NOS: 125169, 125170, 125171, 125172, 125173, 125174, 125175, 125176, 125177, 125178, 125179 were selected from the ORFs having the nucleotide sequence represented by SEQ ID NO: 79139.

The probes having the nucleotide sequence represented by SEQ ID NOS: 125180, 125181, 125182, 125183, 125184, 125185, 125186, 125187, 125188, 125189, 125190 were selected from the ORFs having the nucleotide sequence represented by SEQ ID NO: 79140.

The probes having the nucleotide sequence represented by SEQ ID NOS: 125191, 125192, 125193, 125194, 125195, 125196, 125197, 125198, 125199, 125200, 125201 were selected from the ORFs having the nucleotide sequence represented by SEQ ID NO: 79141.

The probes having the nucleotide sequence represented by SEQ ID NOS: 125202, 125203, 125204, 125205, 125206, 125207, 125208, 125209, 125210, 125211, 125212 were selected from the ORFs having the nucleotide sequence represented by SEQ ID NO: 79142.

The probes having the nucleotide sequence represented by SEQ ID NOS: 125213, 125214, 125215, 125216, 125217, 125218, 125219, 125220, 125221, 125222, 125223 were selected from the ORFs having the nucleotide sequence represented by SEQ ID NO: 79143.

The probes having the nucleotide sequence represented by SEQ ID NOS: 125224, 125225, 125226, 125227, 125228, 125229, 125230, 125231, 125232, 125233, 125234 were selected from the ORFs having the nucleotide sequence represented by SEQ ID NO: 79144.

The probes having the nucleotide sequence represented by SEQ ID NOS: 125235, 125236, 125237, 125238, 125239, 125240, 125241, 125242, 125243, 125244, 125245, 125246, 125247, 125248, 125249, 125250, 125251, 125252, 125253, 125254, 125255, 125256 were selected from the ORFs having the nucleotide sequence represented by SEQ ID NO: 79145.

The probes having the nucleotide sequence represented by SEQ ID NOS: 125257, 125258, 125259, 125260, 125261, 125262, 125263, 125264, 125265, 125266, 125267 were selected from the ORFs having the nucleotide sequence represented by SEQ ID NO: 79146.

The probes having the nucleotide sequence represented by SEQ ID NOS: 125268, 125269, 125270, 125271, 125272, 125273, 125274, 125275, 125276, 125277, 125278 were selected from the ORFs having the nucleotide sequence represented by SEQ ID NO: 79147.

The probes having the nucleotide sequence represented by SEQ ID NOS: 125279, 125280, 125281, 125282, 125283, 125284, 125285, 125286, 125287, 125288, 125289 were selected from the ORFs having the nucleotide sequence represented by SEQ ID NO: 79148.

The probes having the nucleotide sequence represented by SEQ ID NOS: 125290, 125291, 125292, 125293, 125294, 125295, 125296, 125297, 125298, 125299, 125300 were selected from the ORFs having the nucleotide sequence represented by SEQ ID NO: 79149.

The probes having the nucleotide sequence represented by SEQ ID NOS: 125301, 125302, 125303, 125304, 125305, 125306, 125307, 125308, 125309, 125310, 125311 were selected from the ORFs having the nucleotide sequence represented by SEQ ID NO: 79150.

The probes having the nucleotide sequence represented by SEQ ID NOS: 125312, 125313, 125314, 125315, 125316, 125317, 125318, 125319, 125320, 125321, 125322, 125323, 125324, 125325, 125326, 125327, 125328, 125329, 125330, 125331, 125332, 125333 were selected from the ORFs having the nucleotide sequence represented by SEQ ID NO: 79151.

The probes having the nucleotide sequence represented by SEQ ID NOS: 125334, 125335, 125336, 125337, 125338, 125339, 125340, 125341, 125342, 125343, 125344 were selected from the ORFs having the nucleotide sequence represented by SEQ ID NO: 79152.

The probes having the nucleotide sequence represented by SEQ ID NOS: 125345, 125346, 125347, 125348, 125349, 125350, 125351, 125352, 125353, 125354, 125355 were selected from the ORFs having the nucleotide sequence represented by SEQ ID NO: 79153.

The probes having the nucleotide sequence represented by SEQ ID NOS: 125356, 125357, 125358, 125359, 125360, 125361, 125362, 125363, 125364, 125365, 125366 were selected from the ORFs having the nucleotide sequence represented by SEQ ID NO: 79154.

The probes having the nucleotide sequence represented by SEQ ID NOS: 125367, 125368, 125369, 125370, 125371, 125372, 125373, 125374, 125375, 125376, 125377 were selected from the ORFs having the nucleotide sequence represented by SEQ ID NO: 79155.

The probes having the nucleotide sequence represented by SEQ ID NOS: 125378, 125379, 125380, 125381, 125382, 125383, 125384, 125385, 125386, 125387, 125388 were selected from the ORFs having the nucleotide sequence represented by SEQ ID NO: 79156.

The probes having the nucleotide sequence represented by SEQ ID NOS: 125389, 125390, 125391, 125392, 125393, 125394, 125395, 125396, 125397, 125398, 125399 were selected from the ORFs having the nucleotide sequence represented by SEQ ID NO: 79157.

The probes having the nucleotide sequence represented by SEQ ID NOS: 125400, 125401, 125402, 125403, 125404, 125405, 125406, 125407, 125408, 125409, 125410 were selected from the ORFs having the nucleotide sequence represented by SEQ ID NO: 79158.

The probes having the nucleotide sequence represented by SEQ ID NOS: 125411, 125412, 125413, 125414, 125415, 125416, 125417, 125418, 125419, 125420, 125421 were selected from the ORFs having the nucleotide sequence represented by SEQ ID NO: 79159.

The probes having the nucleotide sequence represented by SEQ ID NOS: 125422, 125423, 125424, 125425, 125426, 125427, 125428, 125429, 125430, 125431, 125432 were selected from the ORFs having the nucleotide sequence represented by SEQ ID NO: 79160.

The probes having the nucleotide sequence represented by SEQ ID NOS: 125433, 125434, 125435, 125436, 125437, 125438, 125439, 125440, 125441, 125442, 125443 were selected from the ORFs having the nucleotide sequence represented by SEQ ID NO: 79161.

The probes having the nucleotide sequence represented by SEQ ID NOS: 125444, 125445, 125446, 125447, 125448, 125449, 125450, 125451, 125452, 125453, 125454 were selected from the ORFs having the nucleotide sequence represented by SEQ ID NO: 79162.

The probes having the nucleotide sequence represented by SEQ ID NOS: 125455, 125456, 125457, 125458, 125459, 125460, 125461, 125462, 125463, 125464, 125465 were selected from the ORFs having the nucleotide sequence represented by SEQ ID NO: 79163.

The probes having the nucleotide sequence represented by SEQ ID NOS: 125466, 125467, 125468, 125469, 125470, 125471, 125472, 125473, 125474, 125475, 125476, 125477, 125478, 125479, 125480, 125481, 125482, 125483, 125484, 125485, 125486, 125487 were selected from the ORFs having the nucleotide sequence represented by SEQ ID NO: 79164.

The probes having the nucleotide sequence represented by SEQ ID NOS: 125488, 125489, 125490, 125491, 125492, 125493, 125494, 125495, 125496, 125497, 125498 were selected from the ORFs having the nucleotide sequence represented by SEQ ID NO: 79165.

The probes having the nucleotide sequence represented by SEQ ID NOS: 125499, 125500, 125501, 125502, 125503, 125504, 125505, 125506, 125507, 125508, 125509 were selected from the ORFs having the nucleotide sequence represented by SEQ ID NO: 79166.

The probes having the nucleotide sequence represented by SEQ ID NOS: 125510, 125511, 125512, 125513, 125514, 125515, 125516, 125517, 125518, 125519, 125520 were selected from the ORFs having the nucleotide sequence represented by SEQ ID NO: 79167.

The probes having the nucleotide sequence represented by SEQ ID NOS: 125521, 125522, 125523, 125524, 125525, 125526, 125527, 125528, 125529, 125530, 125531 were selected from the ORFs having the nucleotide sequence represented by SEQ ID NO: 79168.

The probes having the nucleotide sequence represented by SEQ ID NOS: 125532, 125533, 125534, 125535, 125536, 125537, 125538, 125539, 125540, 125541, 125542 were selected from the ORFs having the nucleotide sequence represented by SEQ ID NO: 79169.

The probes having the nucleotide sequence represented by SEQ ID NOS: 125543, 125544, 125545, 125546, 125547, 125548, 125549, 125550, 125551, 125552, 125553 were selected from the ORFs having the nucleotide sequence represented by SEQ ID NO: 79170.

The probes having the nucleotide sequence represented by SEQ ID NOS: 125554, 125555, 125556, 125557, 125558, 125559, 125560, 125561, 125562, 125563, 125564 were selected from the ORFs having the nucleotide sequence represented by SEQ ID NO: 79171.

The probes having the nucleotide sequence represented by SEQ ID NOS: 125565, 125566, 125567, 125568, 125569, 125570, 125571, 125572, 125573, 125574, 125575 were selected from the ORFs having the nucleotide sequence represented by SEQ ID NO: 79172.

The probes having the nucleotide sequence represented by SEQ ID NOS: 125576, 125577, 125578, 125579, 125580, 125581, 125582, 125583, 125584, 125585, 125586 were selected from the ORFs having the nucleotide sequence represented by SEQ ID NO: 79173.

The probes having the nucleotide sequence represented by SEQ ID NOS: 125587, 125588, 125589, 125590, 125591, 125592, 125593, 125594, 125595, 125596, 125597 were selected from the ORFs having the nucleotide sequence represented by SEQ ID NO: 79174.

The probes having the nucleotide sequence represented by SEQ ID NOS: 125598, 125599, 125600, 125601, 125602, 125603, 125604, 125605, 125606, 125607, 125608 were selected from the ORFs having the nucleotide sequence represented by SEQ ID NO: 79175.

The probes having the nucleotide sequence represented by SEQ ID NOS: 125609, 125610, 125611, 125612, 125613, 125614, 125615, 125616, 125617, 125618, 125619 were selected from the ORFs having the nucleotide sequence represented by SEQ ID NO: 79176.

The probes having the nucleotide sequence represented by SEQ ID NOS: 125620, 125621, 125622, 125623, 125624, 125625, 125626, 125627, 125628, 125629, 125630 were selected from the ORFs having the nucleotide sequence represented by SEQ ID NO: 79177.

The probes having the nucleotide sequence represented by SEQ ID NOS: 125631, 125632, 125633, 125634, 125635, 125636, 125637, 125638, 125639, 125640, 125641 were selected from the ORFs having the nucleotide sequence represented by SEQ ID NO: 79178.

The probes having the nucleotide sequence represented by SEQ ID NOS: 125642, 125643, 125644, 125645, 125646, 125647, 125648, 125649, 125650, 125651, 125652 were selected from the ORFs having the nucleotide sequence represented by SEQ ID NO: 79179.

The probes having the nucleotide sequence represented by SEQ ID NOS: 125653, 125654, 125655, 125656, 125657, 125658, 125659, 125660, 125661, 125662, 125663 were selected from the ORFs having the nucleotide sequence represented by SEQ ID NO: 79180.

The probes having the nucleotide sequence represented by SEQ ID NOS: 125664, 125665, 125666, 125667, 125668, 125669, 125670, 125671, 125672, 125673, 125674 were selected from the ORFs having the nucleotide sequence represented by SEQ ID NO: 79181.

The probes having the nucleotide sequence represented by SEQ ID NOS: 125675, 125676, 125677, 125678, 125679, 125680, 125681, 125682, 125683, 125684, 125685 were selected from the ORFs having the nucleotide sequence represented by SEQ ID NO: 79182.

The probes having the nucleotide sequence represented by SEQ ID NOS: 125686, 125687, 125688, 125689, 125690, 125691, 125692, 125693, 125694, 125695, 125696 were selected from the ORFs having the nucleotide sequence represented by SEQ ID NO: 79183.

The probes having the nucleotide sequence represented by SEQ ID NOS: 125697, 125698, 125699, 125700, 125701, 125702, 125703, 125704, 125705, 125706, 125707 were selected from the ORFs having the nucleotide sequence represented by SEQ ID NO: 79184.

The probes having the nucleotide sequence represented by SEQ ID NOS: 125708, 125709, 125710, 125711, 125712, 125713, 125714, 125715, 125716, 125717, 125718 were selected from the ORFs having the nucleotide sequence represented by SEQ ID NO: 79185.

The probes having the nucleotide sequence represented by SEQ ID NOS: 125719, 125720, 125721, 125722, 125723, 125724, 125725, 125726, 125727, 125728, 125729 were selected from the ORFs having the nucleotide sequence represented by SEQ ID NO: 79186.

The probes having the nucleotide sequence represented by SEQ ID NOS: 125730, 125731, 125732, 125733, 125734, 125735, 125736, 125737, 125738, 125739, 125740 were selected from the ORFs having the nucleotide sequence represented by SEQ ID NO: 79187.

The probes having the nucleotide sequence represented by SEQ ID NOS: 125741, 125742, 125743, 125744, 125745, 125746, 125747, 125748, 125749, 125750, 125751 were selected from the ORFs having the nucleotide sequence represented by SEQ ID NO: 79188.

The probes having the nucleotide sequence represented by SEQ ID NOS: 125752, 125753, 125754, 125755, 125756, 125757, 125758, 125759, 125760, 125761, 125762 were selected from the ORFs having the nucleotide sequence represented by SEQ ID NO: 79189.

The probes having the nucleotide sequence represented by SEQ ID NOS: 125763, 125764, 125765, 125766, 125767, 125768, 125769, 125770, 125771, 125772, 125773 were selected from the ORFs having the nucleotide sequence represented by SEQ ID NO: 79190.

The probes having the nucleotide sequence represented by SEQ ID NOS: 125774, 125775, 125776, 125777, 125778, 125779, 125780, 125781, 125782, 125783, 125784 were selected from the ORFs having the nucleotide sequence represented by SEQ ID NO: 79191.

The probes having the nucleotide sequence represented by SEQ ID NOS: 125785, 125786, 125787, 125788, 125789, 125790, 125791, 125792, 125793, 125794, 125795 were selected from the ORFs having the nucleotide sequence represented by SEQ ID NO: 79192.

The probes having the nucleotide sequence represented by SEQ ID NOS: 125796, 125797, 125798, 125799, 125800, 125801, 125802, 125803, 125804, 125805, 125806 were selected from the ORFs having the nucleotide sequence represented by SEQ ID NO: 79193.

The probes having the nucleotide sequence represented by SEQ ID NOS: 125807, 125808, 125809, 125810, 125811, 125812, 125813, 125814, 125815, 125816, 125817 were selected from the ORFs having the nucleotide sequence represented by SEQ ID NO: 79194.

The probes having the nucleotide sequence represented by SEQ ID NOS: 125818, 125819, 125820, 125821, 125822, 125823, 125824, 125825, 125826, 125827, 125828 were selected from the ORFs having the nucleotide sequence represented by SEQ ID NO: 79195.

The probes having the nucleotide sequence represented by SEQ ID NOS: 125829, 125830, 125831, 125832, 125833, 125834, 125835, 125836, 125837, 125838, 125839 were selected from the ORFs having the nucleotide sequence represented by SEQ ID NO: 79196.

The probes having the nucleotide sequence represented by SEQ ID NOS: 125840, 125841, 125842, 125843, 125844, 125845, 125846, 125847, 125848, 125849, 125850 were selected from the ORFs having the nucleotide sequence represented by SEQ ID NO: 79197.

The probes having the nucleotide sequence represented by SEQ ID NOS: 125851, 125852, 125853, 125854, 125855, 125856, 125857, 125858, 125859, 125860, 125861 were selected from the ORFs having the nucleotide sequence represented by SEQ ID NO: 79198.

The probes having the nucleotide sequence represented by SEQ ID NOS: 125862, 125863, 125864, 125865, 125866, 125867, 125868, 125869, 125870, 125871, 125872 were selected from the ORFs having the nucleotide sequence represented by SEQ ID NO: 79199.

The probes having the nucleotide sequence represented by SEQ ID NOS: 125873, 125874, 125875, 125876, 125877, 125878, 125879, 125880, 125881, 125882, 125883 were selected from the ORFs having the nucleotide sequence represented by SEQ ID NO: 79200.

The probes having the nucleotide sequence represented by SEQ ID NOS: 125884, 125885, 125886, 125887, 125888, 125889, 125890, 125891, 125892, 125893, 125894 were selected from the ORFs having the nucleotide sequence represented by SEQ ID NO: 79201.

The probes having the nucleotide sequence represented by SEQ ID NOS: 125895, 125896, 125897, 125898, 125899, 125900, 125901, 125902, 125903, 125904, 125905 were selected from the ORFs having the nucleotide sequence represented by SEQ ID NO: 79202.

The probes having the nucleotide sequence represented by SEQ ID NOS: 125906, 125907, 125908, 125909, 125910, 125911, 125912, 125913, 125914, 125915, 125916 were selected from the ORFs having the nucleotide sequence represented by SEQ ID NO: 79203.

The probes having the nucleotide sequence represented by SEQ ID NOS: 125917, 125918, 125919, 125920, 125921, 125922, 125923, 125924, 125925, 125926, 125927 were selected from the ORFs having the nucleotide sequence represented by SEQ ID NO: 79204.

The probes having the nucleotide sequence represented by SEQ ID NOS: 125928, 125929, 125930, 125931, 125932, 125933, 125934, 125935, 125936, 125937, 125938 were selected from the ORFs having the nucleotide sequence represented by SEQ ID NO: 79205.

The probes having the nucleotide sequence represented by SEQ ID NOS: 125939, 125940, 125941, 125942, 125943, 125944, 125945, 125946, 125947, 125948, 125949 were selected from the ORFs having the nucleotide sequence represented by SEQ ID NO: 79206.

The probes having the nucleotide sequence represented by SEQ ID NOS: 125950, 125951, 125952, 125953, 125954, 125955, 125956, 125957, 125958, 125959, 125960 were selected from the ORFs having the nucleotide sequence represented by SEQ ID NO: 79207.

The probes having the nucleotide sequence represented by SEQ ID NOS: 125961, 125962, 125963, 125964, 125965, 125966, 125967, 125968, 125969, 125970, 125971, 125972, 125973, 125974, 125975, 125976, 125977, 125978, 125979, 125980, 125981, 125982 were selected from the ORFs having the nucleotide sequence represented by SEQ ID NO: 79208.

The probes having the nucleotide sequence represented by SEQ ID NOS: 125983, 125984, 125985, 125986, 125987, 125988, 125989, 125990, 125991, 125992, 125993 were selected from the ORFs having the nucleotide sequence represented by SEQ ID NO: 79209.

The probes having the nucleotide sequence represented by SEQ ID NOS: 125994, 125995, 125996, 125997, 125998, 125999, 126000, 126001, 126002, 126003, 126004 were selected from the ORFs having the nucleotide sequence represented by SEQ ID NO: 79210.

The probes having the nucleotide sequence represented by SEQ ID NOS: 126005, 126006, 126007, 126008, 126009, 126010, 126011, 126012, 126013, 126014, 126015, 126016, 126017, 126018, 126019, 126020, 126021, 126022, 126023, 126024, 126025, 126026 were selected from the ORFs having the nucleotide sequence represented by SEQ ID NO: 79211.

The probes having the nucleotide sequence represented by SEQ ID NOS: 126027, 126028, 126029, 126030, 126031, 126032, 126033, 126034, 126035, 126036, 126037 were selected from the ORFs having the nucleotide sequence represented by SEQ ID NO: 79212.

The probes having the nucleotide sequence represented by SEQ ID NOS: 126038, 126039, 126040, 126041, 126042, 126043, 126044, 126045, 126046, 126047, 126048 were selected from the ORFs having the nucleotide sequence represented by SEQ ID NO: 79213.

The probes having the nucleotide sequence represented by SEQ ID NOS: 126049, 126050, 126051, 126052, 126053, 126054, 126055, 126056, 126057, 126058, 126059 were selected from the ORFs having the nucleotide sequence represented by SEQ ID NO: 79214.

The probes having the nucleotide sequence represented by SEQ ID NOS: 126060, 126061, 126062, 126063, 126064, 126065, 126066, 126067, 126068, 126069, 126070 were selected from the ORFs having the nucleotide sequence represented by SEQ ID NO: 79215.

The probes having the nucleotide sequence represented by SEQ ID NOS: 126071, 126072, 126073, 126074, 126075, 126076, 126077, 126078, 126079, 126080, 126081 were selected from the ORFs having the nucleotide sequence represented by SEQ ID NO: 79216.

The probes having the nucleotide sequence represented by SEQ ID NOS: 126082, 126083, 126084, 126085, 126086, 126087, 126088, 126089, 126090, 126091, 126092 were selected from the ORFs having the nucleotide sequence represented by SEQ ID NO: 79217.

The probes having the nucleotide sequence represented by SEQ ID NOS: 126093, 126094, 126095, 126096, 126097, 126098, 126099, 126100, 126101, 126102, 126103 were selected from the ORFs having the nucleotide sequence represented by SEQ ID NO: 79218.

The probes having the nucleotide sequence represented by SEQ ID NOS: 126104, 126105, 126106, 126107, 126108, 126109, 126110, 126111, 126112, 126113, 126114 were selected from the ORFs having the nucleotide sequence represented by SEQ ID NO: 79219.

The probes having the nucleotide sequence represented by SEQ ID NOS: 126115, 126116, 126117, 126118, 126119, 126120, 126121, 126122, 126123, 126124, 126125, 126126, 126127, 126128, 126129, 126130, 126131, 126132, 126133, 126134, 126135, 126136 were selected from the ORFs having the nucleotide sequence represented by SEQ ID NO: 79220.

The probes having the nucleotide sequence represented by SEQ ID NOS: 126137, 126138, 126139, 126140, 126141, 126142, 126143, 126144, 126145, 126146, 126147 were selected from the ORFs having the nucleotide sequence represented by SEQ ID NO: 79221.

The probes having the nucleotide sequence represented by SEQ ID NOS: 126148, 126149, 126150, 126151, 126152, 126153, 126154, 126155, 126156, 126157, 126158 were selected from the ORFs having the nucleotide sequence represented by SEQ ID NO: 79222.

The probes having the nucleotide sequence represented by SEQ ID NOS: 126159, 126160, 126161, 126162, 126163, 126164, 126165, 126166, 126167, 126168, 126169 were selected from the ORFs having the nucleotide sequence represented by SEQ ID NO: 79223.

The probes having the nucleotide sequence represented by SEQ ID NOS: 126170, 126171, 126172, 126173, 126174, 126175, 126176, 126177, 126178, 126179, 126180 were selected from the ORFs having the nucleotide sequence represented by SEQ ID NO: 79224.

The probes having the nucleotide sequence represented by SEQ ID NOS: 126181, 126182, 126183, 126184, 126185, 126186, 126187, 126188, 126189, 126190, 126191 were selected from the OREs having the nucleotide sequence represented by SEQ ID NO: 79225.

The probes having the nucleotide sequence represented by SEQ ID NOS: 126192, 126193, 126194, 126195, 126196, 126197, 126198, 126199, 126200, 126201, 126202 were selected from the ORFs having the nucleotide sequence represented by SEQ ID NO: 79226.

The probes having the nucleotide sequence represented by SEQ ID NOS: 126203, 126204, 126205, 126206, 126207, 126208, 126209, 126210, 126211, 126212, 126213 were selected from the ORFs having the nucleotide sequence represented by SEQ ID NO: 79227.

The probes having the nucleotide sequence represented by SEQ ID NOS: 126214, 126215, 126216, 126217, 126218, 126219, 126220, 126221, 126222, 126223, 126224, 126225, 126226, 126227, 126228, 126229, 126230, 126231, 126232, 126233, 126234, 126235 were selected from the ORFs having the nucleotide sequence represented by SEQ ID NO: 79228.

The probes having the nucleotide sequence represented by SEQ ID NOS: 126236, 126237, 126238, 126239, 126240, 126241, 126242, 126243, 126244, 126245, 126246 were selected from the ORFs having the nucleotide sequence represented by SEQ ID NO: 79229.

The probes having the nucleotide sequence represented by SEQ ID NOS: 126247, 126248, 126249, 126250, 126251, 126252, 126253, 126254, 126255, 126256, 126257 were selected from the ORFs having the nucleotide sequence represented by SEQ ID NO: 79230.

The probes having the nucleotide sequence represented by SEQ ID NOS: 126258, 126259, 126260, 126261, 126262, 126263, 126264, 126265, 126266, 126267, 126268 were selected from the ORFs having the nucleotide sequence represented by SEQ ID NO: 79231.

The probes having the nucleotide sequence represented by SEQ ID NOS: 126269, 126270, 126271, 126272, 126273, 126274, 126275, 126276, 126277, 126278, 126279, 126280, 126281, 126282, 126283, 126284, 126285, 126286, 126287, 126288, 126289, 126290 were selected from the ORFs having the nucleotide sequence represented by SEQ ID NO: 79232.

The probes having the nucleotide sequence represented by SEQ ID NOS: 126291, 126292, 126293, 126294, 126295, 126296, 126297, 126298, 126299, 126300, 126301 were selected from the ORFs having the nucleotide sequence represented by SEQ ID NO: 79233.

The probes having the nucleotide sequence represented by SEQ ID NOS: 126302, 126303, 126304, 126305, 126306, 126307, 126308, 126309, 126310, 126311, 126312 were selected from the ORFs having the nucleotide sequence represented by SEQ ID NO: 79234.

The probes having the nucleotide sequence represented by SEQ ID NOS: 126313, 126314, 126315, 126316, 126317, 126318, 126319, 126320, 126321, 126322, 126323 were selected from the ORFs having the nucleotide sequence represented by SEQ ID NO: 79235.

The probes having the nucleotide sequence represented by SEQ ID NOS: 126324, 126325, 126326, 126327, 126328, 126329, 126330, 126331, 126332, 126333, 126334 were selected from the ORFs having the nucleotide sequence represented by SEQ ID NO: 79236.

The probes having the nucleotide sequence represented by SEQ ID NOS: 126335, 126336, 126337, 126338, 126339, 126340, 126341, 126342, 126343, 126344, 126345 were selected from the ORFs having the nucleotide sequence represented by SEQ ID NO: 79237.

The probes having the nucleotide sequence represented by SEQ ID NOS: 126346, 126347, 126348, 126349, 126350, 126351, 126352, 126353, 126354, 126355, 126356 were selected from the ORFs having the nucleotide sequence represented by SEQ ID NO: 79238.

The probes having the nucleotide sequence represented by SEQ ID NOS: 126357, 126358, 126359, 126360, 126361, 126362, 126363, 126364, 126365, 126366, 126367 were selected from the ORFs having the nucleotide sequence represented by SEQ ID NO: 79239.

The probes having the nucleotide sequence represented by SEQ ID NOS: 126368, 126369, 126370, 126371, 126372, 126373, 126374, 126375, 126376, 126377, 126378 were selected from the ORFs having the nucleotide sequence represented by SEQ ID NO: 79240.

The probes having the nucleotide sequence represented by SEQ ID NOS: 126379, 126380, 126381, 126382, 126383, 126384, 126385, 126386, 126387, 126388, 126389 were selected from the ORFs having the nucleotide sequence represented by SEQ ID NO: 79241.

The probes having the nucleotide sequence represented by SEQ ID NOS: 126390, 126391, 126392, 126393, 126394, 126395, 126396, 126397, 126398, 126399, 126400 were selected from the ORFs having the nucleotide sequence represented by SEQ ID NO: 79242.

The probes having the nucleotide sequence represented by SEQ ID NOS: 126401, 126402, 126403, 126404, 126405, 126406, 126407, 126408, 126409, 126410, 126411 were selected from the ORFs having the nucleotide sequence represented by SEQ ID NO: 79243.

The probes having the nucleotide sequence represented by SEQ ID NOS: 126412, 126413, 126414, 126415, 126416, 126417, 126418, 126419, 126420, 126421, 126422 were selected from the ORFs having the nucleotide sequence represented by SEQ ID NO: 79244.

The probes having the nucleotide sequence represented by SEQ ID NOS: 126423, 126424, 126425, 126426, 126427, 126428, 126429, 126430, 126431, 126432, 126433 were selected from the ORFs having the nucleotide sequence represented by SEQ ID NO: 79245.

The probes having the nucleotide sequence represented by SEQ ID NOS: 126434, 126435, 126436, 126437, 126438, 126439, 126440, 126441, 126442, 126443, 126444 were selected from the ORFs having the nucleotide sequence represented by SEQ ID NO: 79246.

The probes having the nucleotide sequence represented by SEQ ID NOS: 126445, 126446, 126447, 126448, 126449, 126450, 126451, 126452, 126453, 126454, 126455 were selected from the ORFs having the nucleotide sequence represented by SEQ ID NO: 79247.

The probes having the nucleotide sequence represented by SEQ ID NOS: 126456, 126457, 126458, 126459, 126460, 126461, 126462, 126463, 126464, 126465, 126466 were selected from the ORFs having the nucleotide sequence represented by SEQ ID NO: 79248.

The probes having the nucleotide sequence represented by SEQ ID NOS: 126467, 126468, 126469, 126470, 126471, 126472, 126473, 126474, 126475, 126476, 126477 were selected from the ORFs having the nucleotide sequence represented by SEQ ID NO: 79249.

The probes having the nucleotide sequence represented by SEQ ID NOS: 126478, 126479, 126480, 126481, 126482, 126483, 126484, 126485, 126486, 126487, 126488 were selected from the ORFs having the nucleotide sequence represented by SEQ ID NO: 79250.

The probes having the nucleotide sequence represented by SEQ ID NOS: 126489, 126490, 126491, 126492, 126493, 126494, 126495, 126496, 126497, 126498, 126499 were selected from the ORFs having the nucleotide sequence represented by SEQ ID NO: 79251.

The probes having the nucleotide sequence represented by SEQ ID NOS: 126500, 126501, 126502, 126503, 126504, 126505, 126506, 126507, 126508, 126509, 126510 were selected from the ORFs having the nucleotide sequence represented by SEQ ID NO: 79252.

The probes having the nucleotide sequence represented by SEQ ID NOS: 126511, 126512, 126513, 126514, 126515, 126516, 126517, 126518, 126519, 126520, 126521 were selected from the ORFs having the nucleotide sequence represented by SEQ ID NO: 79253.

The probes having the nucleotide sequence represented by SEQ ID NOS: 126522, 126523, 126524, 126525, 126526, 126527, 126528, 126529, 126530, 126531, 126532 were selected from the ORFs having the nucleotide sequence represented by SEQ ID NO: 79254.

The probes having the nucleotide sequence represented by SEQ ID NOS: 126533, 126534, 126535, 126536, 126537, 126538, 126539, 126540, 126541, 126542, 126543 were selected from the ORFs having the nucleotide sequence represented by SEQ ID NO: 79255.

The probes having the nucleotide sequence represented by SEQ ID NOS: 126544, 126545, 126546, 126547, 126548, 126549, 126550, 126551, 126552, 126553, 126554 were selected from the ORFs having the nucleotide sequence represented by SEQ ID NO: 79256.

The probes having the nucleotide sequence represented by SEQ ID NOS: 126555, 126556, 126557, 126558, 126559, 126560, 126561, 126562, 126563, 126564, 126565 were selected from the ORFs having the nucleotide sequence represented by SEQ ID NO: 79257.

The probes having the nucleotide sequence represented by SEQ ID NOS: 126566, 126567, 126568, 126569, 126570, 126571, 126572, 126573, 126574, 126575, 126576 were selected from the ORFs having the nucleotide sequence represented by SEQ ID NO: 79258.

The probes having the nucleotide sequence represented by SEQ ID NOS: 126577, 126578, 126579, 126580, 126581, 126582, 126583, 126584, 126585, 126586, 126587 were selected from the ORFs having the nucleotide sequence represented by SEQ ID NO: 79259.

The probes having the nucleotide sequence represented by SEQ ID NOS: 126588, 126589, 126590, 126591, 126592, 126593, 126594, 126595, 126596, 126597, 126598 were selected from the ORFs having the nucleotide sequence represented by SEQ ID NO: 79260.

The probes having the nucleotide sequence represented by SEQ ID NOS: 126599, 126600, 126601, 126602, 126603, 126604, 126605, 126606, 126607, 126608, 126609 were selected from the ORFs having the nucleotide sequence represented by SEQ ID NO: 79261.

The probes having the nucleotide sequence represented by SEQ ID NOS: 126610, 126611, 126612, 126613, 126614, 126615, 126616, 126617, 126618, 126619, 126620 were selected from the ORFs having the nucleotide sequence represented by SEQ ID NO: 79262.

The probes having the nucleotide sequence represented by SEQ ID NOS: 126621, 126622, 126623, 126624, 126625, 126626, 126627, 126628, 126629, 126630, 126631 were selected from the ORFs having the nucleotide sequence represented by SEQ ID NO: 79263.

The probes having the nucleotide sequence represented by SEQ ID NOS: 126632, 126633, 126634, 126635, 126636, 126637, 126638, 126639, 126640, 126641, 126642 were selected from the ORFs having the nucleotide sequence represented by SEQ ID NO: 79264.

The probes having the nucleotide sequence represented by SEQ ID NOS: 126643, 126644, 126645, 126646, 126647, 126648, 126649, 126650, 126651, 126652, 126653 were selected from the ORFs having the nucleotide sequence represented by SEQ ID NO: 79265.

The probes having the nucleotide sequence represented by SEQ ID NOS: 126654, 126655, 126656, 126657, 126658, 126659, 126660, 126661, 126662, 126663, 126664 were selected from the ORFs having the nucleotide sequence represented by SEQ ID NO: 79266.

The probes having the nucleotide sequence represented by SEQ ID NOS: 126665, 126666, 126667, 126668, 126669, 126670, 126671, 126672, 126673, 126674, 126675 were selected from the ORFs having the nucleotide sequence represented by SEQ ID NO: 79267.

The probes having the nucleotide sequence represented by SEQ ID NOS: 126676, 126677, 126678, 126679, 126680, 126681, 126682, 126683, 126684, 126685, 126686 were selected from the ORFs having the nucleotide sequence represented by SEQ ID NO: 79268.

The probes having the nucleotide sequence represented by SEQ ID NOS: 126687, 126688, 126689, 126690, 126691, 126692, 126693, 126694, 126695, 126696, 126697 were selected from the ORFs having the nucleotide sequence represented by SEQ ID NO: 79269.

The probes having the nucleotide sequence represented by SEQ ID NOS: 126698, 126699, 126700, 126701, 126702, 126703, 126704, 126705, 126706, 126707, 126708 were selected from the ORFs having the nucleotide sequence represented by SEQ ID NO: 79270.

The probes having the nucleotide sequence represented by SEQ ID NOS: 126709, 126710, 126711, 126712, 126713, 126714, 126715, 126716, 126717, 126718, 126719 were selected from the ORFs having the nucleotide sequence represented by SEQ ID NO: 79271.

The probes having the nucleotide sequence represented by SEQ ID NOS: 126720, 126721, 126722, 126723, 126724, 126725, 126726, 126727, 126728, 126729, 126730 were selected from the ORFs having the nucleotide sequence represented by SEQ ID NO: 79272.

The probes having the nucleotide sequence represented by SEQ ID NOS: 126731, 126732, 126733, 126734, 126735, 126736, 126737, 126738, 126739, 126740, 126741 were selected from the ORFs having the nucleotide sequence represented by SEQ ID NO: 79273.

The probes having the nucleotide sequence represented by SEQ ID NOS: 126742, 126743, 126744, 126745, 126746, 126747, 126748, 126749, 126750, 126751, 126752 were selected from the ORFs having the nucleotide sequence represented by SEQ ID NO: 79274.

The probes having the nucleotide sequence represented by SEQ ID NOS: 126753, 126754, 126755, 126756, 126757, 126758, 126759, 126760, 126761, 126762, 126763 were selected from the ORFs having the nucleotide sequence represented by SEQ ID NO: 79275.

The probes having the nucleotide sequence represented by SEQ ID NOS: 126764, 126765, 126766, 126767, 126768, 126769, 126770, 126771, 126772, 126773, 126774 were selected from the ORFs having the nucleotide sequence represented by SEQ ID NO: 79276.

The probes having the nucleotide sequence represented by SEQ ID NOS: 126775, 126776, 126777, 126778, 126779, 126780, 126781, 126782, 126783, 126784, 126785 were selected from the ORFs having the nucleotide sequence represented by SEQ ID NO: 79277.

The probes having the nucleotide sequence represented by SEQ ID NOS: 126786, 126787, 126788, 126789, 126790, 126791, 126792, 126793, 126794, 126795, 126796 were selected from the ORFs having the nucleotide sequence represented by SEQ ID NO: 79278.

The probes having the nucleotide sequence represented by SEQ ID NOS: 126797, 126798, 126799, 126800, 126801, 126802, 126803, 126804, 126805, 126806, 126807 were selected from the ORFs having the nucleotide sequence represented by SEQ ID NO: 79279.

The probes having the nucleotide sequence represented by SEQ ID NOS: 126808, 126809, 126810, 126811, 126812, 126813, 126814, 126815, 126816, 126817, 126818 were selected from the ORFs having the nucleotide sequence represented by SEQ ID NO: 79280.

The probes having the nucleotide sequence represented by SEQ ID NOS: 126819, 126820, 126821, 126822, 126823, 126824, 126825, 126826, 126827, 126828, 126829 were selected from the ORFs having the nucleotide sequence represented by SEQ ID NO: 79281.

The probes having the nucleotide sequence represented by SEQ ID NOS: 126830, 126831, 126832, 126833, 126834, 126835, 126836, 126837, 126838, 126839, 126840 were selected from the ORFs having the nucleotide sequence represented by SEQ ID NO: 79282.

The probes having the nucleotide sequence represented by SEQ ID NOS: 126841, 126842, 126843, 126844, 126845, 126846, 126847, 126848, 126849, 126850, 126851, 126852, 126853, 126854, 126855, 126856, 126857, 126858, 126859, 126860, 126861, 126862 were selected from the ORFs having the nucleotide sequence represented by SEQ ID NO: 79283.

The probes having the nucleotide sequence represented by SEQ ID NOS: 126863, 126864, 126865, 126866, 126867, 126868, 126869, 126870, 126871, 126872, 126873 were selected from the ORFs having the nucleotide sequence represented by SEQ ID NO: 79284.

The probes having the nucleotide sequence represented by SEQ ID NOS: 126874, 126875, 126876, 126877, 126878, 126879, 126880, 126881, 126882, 126883, 126884 were selected from the ORFs having the nucleotide sequence represented by SEQ ID NO: 79285.

The probes having the nucleotide sequence represented by SEQ ID NOS: 126885, 126886, 126887, 126888, 126889, 126890, 126891, 126892, 126893, 126894, 126895 were selected from the ORFs having the nucleotide sequence represented by SEQ ID NO: 79286.

The probes having the nucleotide sequence represented by SEQ ID NOS: 126896, 126897, 126898, 126899, 126900, 126901, 126902, 126903, 126904, 126905, 126906 were selected from the ORFs having the nucleotide sequence represented by SEQ ID NO: 79287.

The probes having the nucleotide sequence represented by SEQ ID NOS: 126907, 126908, 126909, 126910, 126911, 126912, 126913, 126914, 126915, 126916, 126917 were selected from the ORFs having the nucleotide sequence represented by SEQ ID NO: 79288.

The probes having the nucleotide sequence represented by SEQ ID NOS: 126918, 126919, 126920, 126921, 126922, 126923, 126924, 126925, 126926, 126927, 126928 were selected from the ORFs having the nucleotide sequence represented by SEQ ID NO: 79289.

The probes having the nucleotide sequence represented by SEQ ID NOS: 126929, 126930, 126931, 126932, 126933, 126934, 126935, 126936, 126937, 126938, 126939 were selected from the ORFs having the nucleotide sequence represented by SEQ ID NO: 79290.

The probes having the nucleotide sequence represented by SEQ ID NOS: 126940, 126941, 126942, 126943, 126944, 126945, 126946, 126947, 126948, 126949, 126950 were selected from the ORFs having the nucleotide sequence represented by SEQ ID NO: 79291.

The probes having the nucleotide sequence represented by SEQ ID NOS: 126951, 126952, 126953, 126954, 126955, 126956, 126957, 126958, 126959, 126960, 126961 were selected from the ORFs having the nucleotide sequence represented by SEQ ID NO: 79292.

The probes having the nucleotide sequence represented by SEQ ID NOS: 126962, 126963, 126964, 126965, 126966, 126967, 126968, 126969, 126970, 126971, 126972 were selected from the ORFs having the nucleotide sequence represented by SEQ ID NO: 79293.

The probes having the nucleotide sequence represented by SEQ ID NOS: 126973, 126974, 126975, 126976, 126977, 126978, 126979, 126980, 126981, 126982, 126983 were selected from the ORFs having the nucleotide sequence represented by SEQ ID NO: 79294.

The probes having the nucleotide sequence represented by SEQ ID NOS: 126984, 126985, 126986, 126987, 126988, 126989, 126990, 126991, 126992, 126993, 126994 were selected from the ORFs having the nucleotide sequence represented by SEQ ID NO: 79295.

The probes having the nucleotide sequence represented by SEQ ID NOS: 126995, 126996, 126997, 126998, 126999, 127000, 127001, 127002, 127003, 127004, 127005 were selected from the ORFs having the nucleotide sequence represented by SEQ ID NO: 79296.

The probes having the nucleotide sequence represented by SEQ ID NOS: 127006, 127007, 127008, 127009, 127010, 127011, 127012, 127013, 127014, 127015, 127016 were selected from the ORFs having the nucleotide sequence represented by SEQ ID NO: 79297.

The probes having the nucleotide sequence represented by SEQ ID NOS: 127017, 127018, 127019, 127020, 127021, 127022, 127023, 127024, 127025, 127026, 127027 were selected from the ORFs having the nucleotide sequence represented by SEQ ID NO: 79298.

The probes having the nucleotide sequence represented by SEQ ID NOS: 127028, 127029, 127030, 127031, 127032, 127033, 127034, 127035, 127036, 127037, 127038 were selected from the ORFs having the nucleotide sequence represented by SEQ ID NO: 79299.

The probes having the nucleotide sequence represented by SEQ ID NOS: 127039, 127040, 127041, 127042, 127043, 127044, 127045, 127046, 127047, 127048, 127049 were selected from the ORFs having the nucleotide sequence represented by SEQ ID NO: 79300.

The probes having the nucleotide sequence represented by SEQ ID NOS: 127050, 127051, 127052, 127053, 127054, 127055, 127056, 127057, 127058, 127059, 127060 were selected from the ORFs having the nucleotide sequence represented by SEQ ID NO: 79301.

The probes having the nucleotide sequence represented by SEQ ID NOS: 127061, 127062, 127063, 127064, 127065, 127066, 127067, 127068, 127069, 127070, 127071 were selected from the ORFs having the nucleotide sequence represented by SEQ ID NO: 79302.

The probes having the nucleotide sequence represented by SEQ ID NOS: 127072, 127073, 127074, 127075, 127076, 127077, 127078, 127079, 127080, 127081, 127082 were selected from the ORFs having the nucleotide sequence represented by SEQ ID NO: 79303.

The probes having the nucleotide sequence represented by SEQ ID NOS: 127083, 127084, 127085, 127086, 127087, 127088, 127089, 127090, 127091, 127092, 127093 were selected from the ORFs having the nucleotide sequence represented by SEQ ID NO: 79304.

The probes having the nucleotide sequence represented by SEQ ID NOS: 127094, 127095, 127096, 127097, 127098, 127099, 127100, 127101, 127102, 127103, 127104 were selected from the ORFs having the nucleotide sequence represented by SEQ ID NO: 79305.

The probes having the nucleotide sequence represented by SEQ ID NOS: 127105, 127106, 127107, 127108, 127109, 127110, 127111, 127112, 127113, 127114, 127115 were selected from the ORFs having the nucleotide sequence represented by SEQ ID NO: 79306.

The probes having the nucleotide sequence represented by SEQ ID NOS: 127116, 127117, 127118, 127119, 127120, 127121, 127122, 127123, 127124, 127125, 127126 were selected from the ORFs having the nucleotide sequence represented by SEQ ID NO: 79307.

The probes having the nucleotide sequence represented by SEQ ID NOS: 127127, 127128, 127129, 127130, 127131, 127132, 127133, 127134, 127135, 127136, 127137 were selected from the ORFs having the nucleotide sequence represented by SEQ ID NO: 79308.

The probes having the nucleotide sequence represented by SEQ ID NOS: 127138, 127139, 127140, 127141, 127142, 127143, 127144, 127145, 127146, 127147, 127148 were selected from the ORFs having the nucleotide sequence represented by SEQ ID NO: 79309.

The probes having the nucleotide sequence represented by SEQ ID NOS: 127149, 127150, 127151, 127152, 127153, 127154, 127155, 127156, 127157, 127158, 127159 were selected from the ORFs having the nucleotide sequence represented by SEQ ID NO: 79310.

The probes having the nucleotide sequence represented by SEQ ID NOS: 127160, 127161, 127162, 127163, 127164, 127165, 127166, 127167, 127168, 127169, 127170 were selected from the ORFs having the nucleotide sequence represented by SEQ ID NO: 79311.

The probes having the nucleotide sequence represented by SEQ ID NOS: 127171, 127172, 127173, 127174, 127175, 127176, 127177, 127178, 127179, 127180, 127181 were selected from the ORFs having the nucleotide sequence represented by SEQ ID NO: 79312.

The probes having the nucleotide sequence represented by SEQ ID NOS: 127182, 127183, 127184, 127185, 127186, 127187, 127188, 127189, 127190, 127191, 127192 were selected from the ORFs having the nucleotide sequence represented by SEQ ID NO: 79313.

The probes having the nucleotide sequence represented by SEQ ID NOS: 127193, 127194, 127195, 127196, 127197, 127198, 127199, 127200, 127201, 127202, 127203 were selected from the ORFs having the nucleotide sequence represented by SEQ ID NO: 79314.

The probes having the nucleotide sequence represented by SEQ ID NOS: 127204, 127205, 127206, 127207, 127208, 127209, 127210, 127211, 127212, 127213, 127214 were selected from the ORFs having the nucleotide sequence represented by SEQ ID NO: 79315.

The probes having the nucleotide sequence represented by SEQ ID NOS: 127215, 127216, 127217, 127218, 127219, 127220, 127221, 127222, 127223, 127224, 127225 were selected from the ORFs having the nucleotide sequence represented by SEQ ID NO: 79316.

The probes having the nucleotide sequence represented by SEQ ID NOS: 127226, 127227, 127228, 127229, 127230, 127231, 127232, 127233, 127234, 127235, 127236 were selected from the ORFs having the nucleotide sequence represented by SEQ ID NO: 79317.

The probes having the nucleotide sequence represented by SEQ ID NOS: 127237, 127238, 127239, 127240, 127241, 127242, 127243, 127244, 127245, 127246, 127247 were selected from the ORFs having the nucleotide sequence represented by SEQ ID NO: 79318.

The probes having the nucleotide sequence represented by SEQ ID NOS: 127248, 127249, 127250, 127251, 127252, 127253, 127254, 127255, 127256, 127257, 127258 were selected from the ORFs having the nucleotide sequence represented by SEQ ID NO: 79319.

The probes having the nucleotide sequence represented by SEQ ID NOS: 127259, 127260, 127261, 127262, 127263, 127264, 127265, 127266, 127267, 127268, 127269 were selected from the ORFs having the nucleotide sequence represented by SEQ ID NO: 79320.

The probes having the nucleotide sequence represented by SEQ ID NOS: 127270, 127271, 127272, 127273, 127274, 127275, 127276, 127277, 127278, 127279, 127280 were selected from the ORFs having the nucleotide sequence represented by SEQ ID NO: 79321.

The probes having the nucleotide sequence represented by SEQ ID NOS: 127281, 127282, 127283, 127284, 127285, 127286, 127287, 127288, 127289, 127290, 127291 were selected from the ORFs having the nucleotide sequence represented by SEQ ID NO: 79322.

The probes having the nucleotide sequence represented by SEQ ID NOS: 127292, 127293, 127294, 127295, 127296, 127297, 127298, 127299, 127300, 127301, 127302 were selected from the ORFs having the nucleotide sequence represented by SEQ ID NO: 79323.

The probes having the nucleotide sequence represented by SEQ ID NOS: 127303, 127304, 127305, 127306, 127307, 127308, 127309, 127310, 127311, 127312, 127313 were selected from the ORFs having the nucleotide sequence represented by SEQ ID NO: 79324.

The probes having the nucleotide sequence represented by SEQ ID NOS: 127314, 127315, 127316, 127317, 127318, 127319, 127320, 127321, 127322, 127323, 127324 were selected from the ORFs having the nucleotide sequence represented by SEQ ID NO: 79325.

The probes having the nucleotide sequence represented by SEQ ID NOS: 127325, 127326, 127327, 127328, 127329, 127330, 127331, 127332, 127333, 127334, 127335 were selected from the ORFs having the nucleotide sequence represented by SEQ ID NO: 79326.

The probes having the nucleotide sequence represented by SEQ ID NOS: 127336, 127337, 127338, 127339, 127340, 127341, 127342, 127343, 127344, 127345, 127346 were selected from the ORFs having the nucleotide sequence represented by SEQ ID NO: 79327.

The probes having the nucleotide sequence represented by SEQ ID NOS: 127347, 127348, 127349, 127350, 127351, 127352, 127353, 127354, 127355, 127356, 127357 were selected from the ORFs having the nucleotide sequence represented by SEQ ID NO: 79328.

The probes having the nucleotide sequence represented by SEQ ID NOS: 127358, 127359, 127360, 127361, 127362, 127363, 127364, 127365, 127366, 127367, 127368 were selected from the ORFs having the nucleotide sequence represented by SEQ ID NO: 79329.

The probes having the nucleotide sequence represented by SEQ ID NOS: 127369, 127370, 127371, 127372, 127373, 127374, 127375, 127376, 127377, 127378, 127379 were selected from the ORFs having the nucleotide sequence represented by SEQ ID NO: 79330.

The probes having the nucleotide sequence represented by SEQ ID NOS: 127380, 127381, 127382, 127383, 127384, 127385, 127386, 127387, 127388, 127389, 127390 were selected from the ORFs having the nucleotide sequence represented by SEQ ID NO: 79331.

The probes having the nucleotide sequence represented by SEQ ID NOS: 127391, 127392, 127393, 127394, 127395, 127396, 127397, 127398, 127399, 127400, 127401 were selected from the ORFs having the nucleotide sequence represented by SEQ ID NO: 79332.

The probes having the nucleotide sequence represented by SEQ ID NOS: 127402, 127403, 127404, 127405, 127406, 127407, 127408, 127409, 127410, 127411, 127412 were selected from the ORFs having the nucleotide sequence represented by SEQ ID NO: 79333.

The probes having the nucleotide sequence represented by SEQ ID NOS: 127413, 127414, 127415, 127416, 127417, 127418, 127419, 127420, 127421, 127422, 127423 were selected from the ORFs having the nucleotide sequence represented by SEQ ID NO: 79334.

The probes having the nucleotide sequence represented by SEQ ID NOS: 127424, 127425, 127426, 127427, 127428, 127429, 127430, 127431, 127432, 127433, 127434 were selected from the ORFs having the nucleotide sequence represented by SEQ ID NO: 79335.

The probes having the nucleotide sequence represented by SEQ ID NOS: 127435, 127436, 127437, 127438, 127439, 127440, 127441, 127442, 127443, 127444, 127445 were selected from the ORFs having the nucleotide sequence represented by SEQ ID NO: 79336.

The probes having the nucleotide sequence represented by SEQ ID NOS: 127446, 127447, 127448, 127449, 127450, 127451, 127452, 127453, 127454, 127455, 127456 were selected from the ORFs having the nucleotide sequence represented by SEQ ID NO: 79337.

The probes having the nucleotide sequence represented by SEQ ID NOS: 127457, 127458, 127459, 127460, 127461, 127462, 127463, 127464, 127465, 127466, 127467 were selected from the ORFs having the nucleotide sequence represented by SEQ ID NO: 79338.

The probes having the nucleotide sequence represented by SEQ ID NOS: 127468, 127469, 127470, 127471, 127472, 127473, 127474, 127475, 127476, 127477, 127478 were selected from the ORFs having the nucleotide sequence represented by SEQ ID NO: 79339.

The probes having the nucleotide sequence represented by SEQ ID NOS: 127479, 127480, 127481, 127482, 127483, 127484, 127485, 127486, 127487, 127488, 127489 were selected from the ORFs having the nucleotide sequence represented by SEQ ID NO: 79340.

The probes having the nucleotide sequence represented by SEQ ID NOS: 127490, 127491, 127492, 127493, 127494, 127495, 127496, 127497, 127498, 127499, 127500 were selected from the ORFs having the nucleotide sequence represented by SEQ ID NO: 79341.

The probes having the nucleotide sequence represented by SEQ ID NOS: 127501, 127502, 127503, 127504, 127505, 127506, 127507, 127508, 127509, 127510, 127511 were selected from the ORFs having the nucleotide sequence represented by SEQ ID NO: 79342.

The probes having the nucleotide sequence represented by SEQ ID NOS: 127512, 127513, 127514, 127515, 127516, 127517, 127518, 127519, 127520, 127521, 127522 were selected from the ORFs having the nucleotide sequence represented by SEQ ID NO: 79343.

The probes having the nucleotide sequence represented by SEQ ID NOS: 127523, 127524, 127525, 127526, 127527, 127528, 127529, 127530, 127531, 127532, 127533 were selected from the ORFs having the nucleotide sequence represented by SEQ ID NO: 79344.

The probes having the nucleotide sequence represented by SEQ ID NOS: 127534, 127535, 127536, 127537, 127538, 127539, 127540, 127541, 127542, 127543, 127544 were selected from the ORFs having the nucleotide sequence represented by SEQ ID NO: 79345.

The probes having the nucleotide sequence represented by SEQ ID NOS: 127545, 127546, 127547, 127548, 127549, 127550, 127551, 127552, 127553, 127554, 127555 were selected from the ORFs having the nucleotide sequence represented by SEQ ID NO: 79346.

The probes having the nucleotide sequence represented by SEQ ID NOS: 127556, 127557, 127558, 127559, 127560, 127561, 127562, 127563, 127564, 127565, 127566 were selected from the ORFs having the nucleotide sequence represented by SEQ ID NO: 79347.

The probes having the nucleotide sequence represented by SEQ ID NOS: 127567, 127568, 127569, 127570, 127571, 127572, 127573, 127574, 127575, 127576, 127577 were selected from the ORFs having the nucleotide sequence represented by SEQ ID NO: 79348.

The probes having the nucleotide sequence represented by SEQ ID NOS: 127578, 127579, 127580, 127581, 127582, 127583, 127584, 127585, 127586, 127587, 127588 were selected from the ORFs having the nucleotide sequence represented by SEQ ID NO: 79349.

The probes having the nucleotide sequence represented by SEQ ID NOS: 127589, 127590, 127591, 127592, 127593, 127594, 127595, 127596, 127597, 127598, 127599 were selected from the ORFs having the nucleotide sequence represented by SEQ ID NO: 79350.

The probes having the nucleotide sequence represented by SEQ ID NOS: 127600, 127601, 127602, 127603, 127604, 127605, 127606, 127607, 127608, 127609, 127610, 127611, 127612, 127613, 127614, 127615, 127616, 127617, 127618, 127619, 127620, 127621 were selected from the ORFs having the nucleotide sequence represented by SEQ ID NO: 79351.

The probes having the nucleotide sequence represented by SEQ ID NOS: 127622, 127623, 127624, 127625, 127626, 127627, 127628, 127629, 127630, 127631, 127632 were selected from the ORFs having the nucleotide sequence represented by SEQ ID NO: 79352.

The probes having the nucleotide sequence represented by SEQ ID NOS: 127633, 127634, 127635, 127636, 127637, 127638, 127639, 127640, 127641, 127642, 127643 were selected from the ORFs having the nucleotide sequence represented by SEQ ID NO: 79353.

The probes having the nucleotide sequence represented by SEQ ID NOS: 127644, 127645, 127646, 127647, 127648, 127649, 127650, 127651, 127652, 127653, 127654 were selected from the ORFs having the nucleotide sequence represented by SEQ ID NO: 79354.

The probes having the nucleotide sequence represented by SEQ ID NOS: 127655, 127656, 127657, 127658, 127659, 127660, 127661, 127662, 127663, 127664, 127665 were selected from the ORFs having the nucleotide sequence represented by SEQ ID NO: 79355.

The probes having the nucleotide sequence represented by SEQ ID NOS: 127666, 127667, 127668, 127669, 127670, 127671, 127672, 127673, 127674, 127675, 127676 were selected from the ORFs having the nucleotide sequence represented by SEQ ID NO: 79356.

The probes having the nucleotide sequence represented by SEQ ID NOS: 127677, 127678, 127679, 127680, 127681, 127682, 127683, 127684, 127685, 127686, 127687 were selected from the ORFs having the nucleotide sequence represented by SEQ ID NO: 79357.

The probes having the nucleotide sequence represented by SEQ ID NOS: 127688, 127689, 127690, 127691, 127692, 127693, 127694, 127695, 127696, 127697, 127698 were selected from the ORFs having the nucleotide sequence represented by SEQ ID NO: 79358.

The probes having the nucleotide sequence represented by SEQ ID NOS: 127699, 127700, 127701, 127702, 127703, 127704, 127705, 127706, 127707, 127708, 127709 were selected from the ORFs having the nucleotide sequence represented by SEQ ID NO: 79359.

The probes having the nucleotide sequence represented by SEQ ID NOS: 127710, 127711, 127712, 127713, 127714, 127715, 127716, 127717, 127718, 127719, 127720 were selected from the ORFs having the nucleotide sequence represented by SEQ ID NO: 79360.

The probes having the nucleotide sequence represented by SEQ ID NOS: 127721, 127722, 127723, 127724, 127725, 127726, 127727, 127728, 127729, 127730, 127731 were selected from the ORFs having the nucleotide sequence represented by SEQ ID NO: 79361.

The probes having the nucleotide sequence represented by SEQ ID NOS: 127732, 127733, 127734, 127735, 127736, 127737, 127738, 127739, 127740, 127741, 127742 were selected from the ORFs having the nucleotide sequence represented by SEQ ID NO: 79362.

The probes having the nucleotide sequence represented by SEQ ID NOS: 127743, 127744, 127745, 127746, 127747, 127748, 127749, 127750, 127751, 127752, 127753 were selected from the ORFs having the nucleotide sequence represented by SEQ ID NO: 79363.

The probes having the nucleotide sequence represented by SEQ ID NOS: 127754, 127755, 127756, 127757, 127758, 127759, 127760, 127761, 127762, 127763, 127764 were selected from the ORFs having the nucleotide sequence represented by SEQ ID NO: 79364.

The probes having the nucleotide sequence represented by SEQ ID NOS: 127765, 127766, 127767, 127768, 127769, 127770, 127771, 127772, 127773, 127774, 127775 were selected from the ORFs having the nucleotide sequence represented by SEQ ID NO: 79365.

The probes having the nucleotide sequence represented by SEQ ID NOS: 127776, 127777, 127778, 127779, 127780, 127781, 127782, 127783, 127784, 127785, 127786 were selected from the ORFs having the nucleotide sequence represented by SEQ ID NO: 79366.

The probes having the nucleotide sequence represented by SEQ ID NOS: 127787, 127788, 127789, 127790, 127791, 127792, 127793, 127794, 127795, 127796, 127797 were selected from the ORFs having the nucleotide sequence represented by SEQ ID NO: 79367.

The probes having the nucleotide sequence represented by SEQ ID NOS: 127798, 127799, 127800, 127801, 127802, 127803, 127804, 127805, 127806, 127807, 127808 were selected from the ORFs having the nucleotide sequence represented by SEQ ID NO: 79368.

The probes having the nucleotide sequence represented by SEQ ID NOS: 127809, 127810, 127811, 127812, 127813, 127814, 127815, 127816, 127817, 127818, 127819 were selected from the ORFs having the nucleotide sequence represented by SEQ ID NO: 79369.

The probes having the nucleotide sequence represented by SEQ ID NOS: 127820, 127821, 127822, 127823, 127824, 127825, 127826, 127827, 127828, 127829, 127830 were selected from the ORFs having the nucleotide sequence represented by SEQ ID NO: 79370.

The probes having the nucleotide sequence represented by SEQ ID NOS: 127831, 127832, 127833, 127834, 127835, 127836, 127837, 127838, 127839, 127840, 127841 were selected from the ORFs having the nucleotide sequence represented by SEQ ID NO: 79371.

The probes having the nucleotide sequence represented by SEQ ID NOS: 127842, 127843, 127844, 127845, 127846, 127847, 127848, 127849, 127850, 127851, 127852 were selected from the ORFs having the nucleotide sequence represented by SEQ ID NO: 79372.

The probes having the nucleotide sequence represented by SEQ ID NOS: 127853, 127854, 127855, 127856, 127857, 127858, 127859, 127860, 127861, 127862, 127863 were selected from the ORFs having the nucleotide sequence represented by SEQ ID NO: 79373.

The probes having the nucleotide sequence represented by SEQ ID NOS: 127864, 127865, 127866, 127867, 127868, 127869, 127870, 127871, 127872, 127873, 127874 were selected from the ORFs having the nucleotide sequence represented by SEQ ID NO: 79374.

The probes having the nucleotide sequence represented by SEQ ID NOS: 127875, 127876, 127877, 127878, 127879, 127880, 127881, 127882, 127883, 127884, 127885 were selected from the ORFs having the nucleotide sequence represented by SEQ ID NO: 79375.

The probes having the nucleotide sequence represented by SEQ ID NOS: 127886, 127887, 127888, 127889, 127890, 127891, 127892, 127893, 127894, 127895, 127896 were selected from the ORFs having the nucleotide sequence represented by SEQ ID NO: 79376.

The probes having the nucleotide sequence represented by SEQ ID NOS: 127897, 127898, 127899, 127900, 127901, 127902, 127903, 127904, 127905, 127906, 127907 were selected from the ORFs having the nucleotide sequence represented by SEQ ID NO: 79377.

The probes having the nucleotide sequence represented by SEQ ID NOS: 127908, 127909, 127910, 127911, 127912, 127913, 127914, 127915, 127916, 127917, 127918 were selected from the ORFs having the nucleotide sequence represented by SEQ ID NO: 79378.

The probes having the nucleotide sequence represented by SEQ ID NOS: 127919, 127920, 127921, 127922, 127923, 127924, 127925, 127926, 127927, 127928, 127929 were selected from the ORFs having the nucleotide sequence represented by SEQ ID NO: 79379.

The probes having the nucleotide sequence represented by SEQ ID NOS: 127930, 127931, 127932, 127933, 127934, 127935, 127936, 127937, 127938, 127939, 127940 were selected from the ORFs having the nucleotide sequence represented by SEQ ID NO: 79380.

The probes having the nucleotide sequence represented by SEQ ID NOS: 127941, 127942, 127943, 127944, 127945, 127946, 127947, 127948, 127949, 127950, 127951 were selected from the ORFs having the nucleotide sequence represented by SEQ ID NO: 79381.

The probes having the nucleotide sequence represented by SEQ ID NOS: 127952, 127953, 127954, 127955, 127956, 127957, 127958, 127959, 127960, 127961, 127962, 127963, 127964, 127965, 127966, 127967, 127968, 127969, 127970, 127971, 127972, 127973 were selected from the ORFs having the nucleotide sequence represented by SEQ ID NO: 79382.

The probes having the nucleotide sequence represented by SEQ ID NOS: 127974, 127975, 127976, 127977, 127978, 127979, 127980, 127981, 127982, 127983, 127984 were selected from the ORFs having the nucleotide sequence represented by SEQ ID NO: 79383.

The probes having the nucleotide sequence represented by SEQ ID NOS: 127985, 127986, 127987, 127988, 127989, 127990, 127991, 127992, 127993, 127994, 127995 were selected from the ORFs having the nucleotide sequence represented by SEQ ID NO: 79384.

The probes having the nucleotide sequence represented by SEQ ID NOS: 127996, 127997, 127998, 127999, 128000, 128001, 128002, 128003, 128004, 128005, 128006 were selected from the ORFs having the nucleotide sequence represented by SEQ ID NO: 79385.

The probes having the nucleotide sequence represented by SEQ ID NOS: 128007, 128008, 128009, 128010, 128011, 128012, 128013, 128014, 128015, 128016, 128017 were selected from the ORFs having the nucleotide sequence represented by SEQ ID NO: 79386.

The probes having the nucleotide sequence represented by SEQ ID NOS: 128018, 128019, 128020, 128021, 128022, 128023, 128024, 128025, 128026, 128027, 128028 were selected from the ORFs having the nucleotide sequence represented by SEQ ID NO: 79387.

The probes having the nucleotide sequence represented by SEQ ID NOS: 128029, 128030, 128031, 128032, 128033, 128034, 128035, 128036, 128037, 128038, 128039 were selected from the ORFs having the nucleotide sequence represented by SEQ ID NO: 79388.

The probes having the nucleotide sequence represented by SEQ ID NOS: 128040, 128041, 128042, 128043, 128044, 128045, 128046, 128047, 128048, 128049, 128050 were selected from the ORFs having the nucleotide sequence represented by SEQ ID NO: 79389.

The probes having the nucleotide sequence represented by SEQ ID NOS: 128051, 128052, 128053, 128054, 128055, 128056, 128057, 128058, 128059, 128060, 128061 were selected from the ORFs having the nucleotide sequence represented by SEQ ID NO: 79390.

The probes having the nucleotide sequence represented by SEQ ID NOS: 128062, 128063, 128064, 128065, 128066, 128067, 128068, 128069, 128070, 128071, 128072 were selected from the ORFs having the nucleotide sequence represented by SEQ ID NO: 79391.

The probes having the nucleotide sequence represented by SEQ ID NOS: 128073, 128074, 128075, 128076, 128077, 128078, 128079, 128080, 128081, 128082, 128083 were selected from the ORFs having the nucleotide sequence represented by SEQ ID NO: 79392.

The probes having the nucleotide sequence represented by SEQ ID NOS: 128084, 128085, 128086, 128087, 128088, 128089, 128090, 128091, 128092, 128093, 128094 were selected from the ORFs having the nucleotide sequence represented by SEQ ID NO: 79393.

The probes having the nucleotide sequence represented by SEQ ID NOS: 128095, 128096, 128097, 128098, 128099, 128100, 128101, 128102, 128103, 128104, 128105 were selected from the ORFs having the nucleotide sequence represented by SEQ ID NO: 79394.

The probes having the nucleotide sequence represented by SEQ ID NOS: 128106, 128107, 128108, 128109, 128110, 128111, 128112, 128113, 128114, 128115 were selected from the ORFs having the nucleotide sequence represented by SEQ ID NO: 79395.

The probes having the nucleotide sequence represented by SEQ ID NOS: 128116, 128117, 128118, 128119, 128120, 128121, 128122, 128123, 128124, 128125, 128126 were selected from the ORFs having the nucleotide sequence represented by SEQ ID NO: 79396.

The probes having the nucleotide sequence represented by SEQ ID NOS: 128127, 128128, 128129, 128130, 128131, 128132, 128133, 128134, 128135, 128136, 128137 were selected from the ORFs having the nucleotide sequence represented by SEQ ID NO: 79397.

The probes having the nucleotide sequence represented by SEQ ID NOS: 128138, 128139, 128140, 128141, 128142, 128143, 128144, 128145, 128146, 128147, 128148 were selected from the ORFs having the nucleotide sequence represented by SEQ ID NO: 79398.

The probes having the nucleotide sequence represented by SEQ ID NOS: 128149, 128150, 128151, 128152, 128153, 128154, 128155, 128156, 128157, 128158, 128159 were selected from the ORFs having the nucleotide sequence represented by SEQ ID NO: 79399.

The probes having the nucleotide sequence represented by SEQ ID NOS: 128160, 128161, 128162, 128163, 128164, 128165, 128166, 128167, 128168, 128169, 128170 were selected from the ORFs having the nucleotide sequence represented by SEQ ID NO: 79400.

The probes having the nucleotide sequence represented by SEQ ID NOS: 128171, 128172, 128173, 128174, 128175, 128176, 128177, 128178, 128179, 128180, 128181 were selected from the ORFs having the nucleotide sequence represented by SEQ ID NO: 79401.

The probes having the nucleotide sequence represented by SEQ ID NOS: 128182, 128183, 128184, 128185, 128186, 128187, 128188, 128189, 128190, 128191, 128192 were selected from the ORFs having the nucleotide sequence represented by SEQ ID NO: 79402.

The probes having the nucleotide sequence represented by SEQ ID NOS: 128193, 128194, 128195, 128196, 128197, 128198, 128199, 128200, 128201, 128202, 128203 were selected from the ORFs having the nucleotide sequence represented by SEQ ID NO: 79403.

The probes having the nucleotide sequence represented by SEQ ID NOS: 128204, 128205, 128206, 128207, 128208, 128209, 128210, 128211, 128212, 128213, 128214 were selected from the ORFs having the nucleotide sequence represented by SEQ ID NO: 79404.

The probes having the nucleotide sequence represented by SEQ ID NOS: 128215, 128216, 128217, 128218, 128219, 128220, 128221, 128222, 128223, 128224, 128225 were selected from the ORFs having the nucleotide sequence represented by SEQ ID NO: 79405.

The probes having the nucleotide sequence represented by SEQ ID NOS: 128226, 128227, 128228, 128229, 128230, 128231, 128232, 128233, 128234, 128235, 128236 were selected from the ORFs having the nucleotide sequence represented by SEQ ID NO: 79406.

The probes having the nucleotide sequence represented by SEQ ID NOS: 128237, 128238, 128239, 128240, 128241, 128242, 128243, 128244, 128245, 128246, 128247 were selected from the ORFs having the nucleotide sequence represented by SEQ ID NO: 79407.

The probes having the nucleotide sequence represented by SEQ ID NOS: 128248, 128249, 128250, 128251, 128252, 128253, 128254, 128255, 128256, 128257, 128258 were selected from the ORFs having the nucleotide sequence represented by SEQ ID NO: 79408.

The probes having the nucleotide sequence represented by SEQ ID NOS: 128259, 128260, 128261, 128262, 128263, 128264, 128265, 128266, 128267, 128268, 128269 were selected from the ORFs having the nucleotide sequence represented by SEQ ID NO: 79409.

The probes having the nucleotide sequence represented by SEQ ID NOS: 128270, 128271, 128272, 128273, 128274, 128275, 128276, 128277, 128278, 128279, 128280 were selected from the ORFs having the nucleotide sequence represented by SEQ ID NO: 79410.

The probes having the nucleotide sequence represented by SEQ ID NOS: 128281, 128282, 128283, 128284, 128285, 128286, 128287, 128288, 128289, 128290, 128291 were selected from the ORFs having the nucleotide sequence represented by SEQ ID NO: 79411.

The probes having the nucleotide sequence represented by SEQ ID NOS: 128292, 128293, 128294, 128295, 128296, 128297, 128298, 128299, 128300, 128301, 128302 were selected from the ORFs having the nucleotide sequence represented by SEQ ID NO: 79412.

The probes having the nucleotide sequence represented by SEQ ID NOS: 128303, 128304, 128305, 128306, 128307, 128308, 128309, 128310, 128311, 128312, 128313 were selected from the ORFs having the nucleotide sequence represented by SEQ ID NO: 79413.

The probes having the nucleotide sequence represented by SEQ ID NOS: 128314, 128315, 128316, 128317, 128318, 128319, 128320, 128321, 128322, 128323, 128324 were selected from the ORFs having the nucleotide sequence represented by SEQ ID NO: 79414.

The probes having the nucleotide sequence represented by SEQ ID NOS: 128325, 128326, 128327, 128328, 128329, 128330, 128331, 128332, 128333, 128334, 128335 were selected from the ORFs having the nucleotide sequence represented by SEQ ID NO: 79415.

The probes having the nucleotide sequence represented by SEQ ID NOS: 128336, 128337, 128338, 128339, 128340, 128341, 128342, 128343, 128344, 128345, 128346 were selected from the ORFs having the nucleotide sequence represented by SEQ ID NO: 79416.

The probes having the nucleotide sequence represented by SEQ ID NOS: 128347, 128348, 128349, 128350, 128351, 128352, 128353, 128354, 128355 were selected from the ORFs having the nucleotide sequence represented by SEQ ID NO: 79417.

The probes having the nucleotide sequence represented by SEQ ID NOS: 128356, 128357, 128358, 128359, 128360, 128361, 128362, 128363, 128364, 128365, 128366 were selected from the ORFs having the nucleotide sequence represented by SEQ ID NO: 79418.

The probes having the nucleotide sequence represented by SEQ ID NOS: 128367, 128368, 128369, 128370, 128371, 128372, 128373, 128374, 128375, 128376, 128377 were selected from the ORFs having the nucleotide sequence represented by SEQ ID NO: 79419.

The probes having the nucleotide sequence represented by SEQ ID NOS: 128378, 128379, 128380, 128381, 128382, 128383, 128384, 128385, 128386, 128387, 128388 were selected from the ORFs having the nucleotide sequence represented by SEQ ID NO: 79420.

The probes having the nucleotide sequence represented by SEQ ID NOS: 128389, 128390, 128391, 128392, 128393, 128394, 128395, 128396, 128397, 128398, 128399 were selected from the ORFs having the nucleotide sequence represented by SEQ ID NO: 79421.

The probes having the nucleotide sequence represented by SEQ ID NOS: 128400, 128401, 128402, 128403, 128404, 128405, 128406, 128407, 128408, 128409, 128410 were selected from the ORFs having the nucleotide sequence represented by SEQ ID NO: 79422.

The probes having the nucleotide sequence represented by SEQ ID NOS: 128411, 128412, 128413, 128414, 128415, 128416, 128417, 128418, 128419, 128420, 128421 were selected from the ORFs having the nucleotide sequence represented by SEQ ID NO: 79423.

The probes having the nucleotide sequence represented by SEQ ID NOS: 128422, 128423, 128424, 128425, 128426, 128427, 128428, 128429, 128430, 128431, 128432 were selected from the ORFs having the nucleotide sequence represented by SEQ ID NO: 79424.

The probes having the nucleotide sequence represented by SEQ ID NOS: 128433, 128434, 128435, 128436, 128437, 128438, 128439, 128440, 128441, 128442, 128443 were selected from the ORFs having the nucleotide sequence represented by SEQ ID NO: 79425.

The probes having the nucleotide sequence represented by SEQ ID NOS: 128444, 128445, 128446, 128447, 128448, 128449, 128450, 128451, 128452, 128453, 128454, 128455, 128456, 128457, 128458, 128459, 128460, 128461, 128462, 128463, 128464, 128465 were selected from the ORFs having the nucleotide sequence represented by SEQ ID NO: 79426.

The probes having the nucleotide sequence represented by SEQ ID NOS: 128466, 128467, 128468, 128469, 128470, 128471, 128472, 128473, 128474, 128475, 128476, 128477, 128478, 128479, 128480, 128481, 128482, 128483, 128484, 128485, 128486, 128487 were selected from the ORFs having the nucleotide sequence represented by SEQ ID NO: 79427.

The probes having the nucleotide sequence represented by SEQ ID NOS: 128488, 128489, 128490, 128491, 128492, 128493, 128494, 128495, 128496, 128497, 128498 were selected from the ORFs having the nucleotide sequence represented by SEQ ID NO: 79428.

The probes having the nucleotide sequence represented by SEQ ID NOS: 128499, 128500, 128501, 128502, 128503, 128504, 128505, 128506, 128507, 128508, 128509 were selected from the ORFs having the nucleotide sequence represented by SEQ ID NO: 79429.

The probes having the nucleotide sequence represented by SEQ ID NOS: 128510, 128511, 128512, 128513, 128514, 128515, 128516, 128517, 128518, 128519, 128520 were selected from the ORFs having the nucleotide sequence represented by SEQ ID NO: 79430.

The probes having the nucleotide sequence represented by SEQ ID NOS: 128521, 128522, 128523, 128524, 128525, 128526, 128527, 128528, 128529, 128530, 128531 were selected from the ORFs having the nucleotide sequence represented by SEQ ID NO: 79431.

The probes having the nucleotide sequence represented by SEQ ID NOS: 128532, 128533, 128534, 128535, 128536, 128537, 128538, 128539, 128540, 128541, 128542 were selected from the ORFs having the nucleotide sequence represented by SEQ ID NO: 79432.

The probes having the nucleotide sequence represented by SEQ ID NOS: 128543, 128544, 128545, 128546, 128547, 128548, 128549, 128550, 128551, 128552, 128553 were selected from the ORFs having the nucleotide sequence represented by SEQ ID NO: 79433.

The probes having the nucleotide sequence represented by SEQ ID NOS: 128554, 128555, 128556, 128557, 128558, 128559, 128560, 128561, 128562, 128563, 128564 were selected from the ORFs having the nucleotide sequence represented by SEQ ID NO: 79434.

The probes having the nucleotide sequence represented by SEQ ID NOS: 128565, 128566, 128567, 128568, 128569, 128570, 128571, 128572, 128573, 128574, 128575 were selected from the ORFs having the nucleotide sequence represented by SEQ ID NO: 79435.

The probes having the nucleotide sequence represented by SEQ ID NOS: 128576, 128577, 128578, 128579, 128580, 128581, 128582, 128583, 128584, 128585, 128586, 128587, 128588, 128589, 128590, 128591, 128592, 128593, 128594, 128595, 128596, 128597 were selected from the ORFs having the nucleotide sequence represented by SEQ ID NO: 79436.

The probes having the nucleotide sequence represented by SEQ ID NOS: 128598, 128599, 128600, 128601, 128602, 128603, 128604, 128605, 128606, 128607, 128608 were selected from the ORFs having the nucleotide sequence represented by SEQ ID NO: 79437.

The probes having the nucleotide sequence represented by SEQ ID NOS: 128609, 128610, 128611, 128612, 128613, 128614, 128615, 128616, 128617, 128618, 128619 were selected from the ORFs having the nucleotide sequence represented by SEQ ID NO: 79438.

The probes having the nucleotide sequence represented by SEQ ID NOS: 128620, 128621, 128622, 128623, 128624, 128625, 128626, 128627, 128628, 128629, 128630 were selected from the ORFs having the nucleotide sequence represented by SEQ ID NO: 79439.

The probes having the nucleotide sequence represented by SEQ ID NOS: 128631, 128632, 128633, 128634, 128635, 128636, 128637, 128638, 128639, 128640, 128641 were selected from the ORFs having the nucleotide sequence represented by SEQ ID NO: 79440.

The probes having the nucleotide sequence represented by SEQ ID NOS: 128642, 128643, 128644, 128645, 128646, 128647, 128648, 128649, 128650, 128651, 128652 were selected from the ORFs having the nucleotide sequence represented by SEQ ID NO: 79441.

The probes having the nucleotide sequence represented by SEQ ID NOS: 128653, 128654, 128655, 128656, 128657, 128658, 128659, 128660, 128661, 128662, 128663 were selected from the ORFs having the nucleotide sequence represented by SEQ ID NO: 79442.

The probes having the nucleotide sequence represented by SEQ ID NOS: 128664, 128665, 128666, 128667, 128668, 128669, 128670, 128671, 128672, 128673, 128674 were selected from the ORFs having the nucleotide sequence represented by SEQ ID NO: 79443.

The probes having the nucleotide sequence represented by SEQ ID NOS: 128675, 128676, 128677, 128678, 128679, 128680, 128681, 128682, 128683, 128684, 128685 were selected from the ORFs having the nucleotide sequence represented by SEQ ID NO: 79444.

The probes having the nucleotide sequence represented by SEQ ID NOS: 128686, 128687, 128688, 128689, 128690, 128691, 128692, 128693, 128694, 128695, 128696 were selected from the ORFs having the nucleotide sequence represented by SEQ ID NO: 79445.

The probes having the nucleotide sequence represented by SEQ ID NOS: 128697, 128698, 128699, 128700, 128701, 128702, 128703, 128704, 128705, 128706, 128707 were selected from the ORFs having the nucleotide sequence represented by SEQ ID NO: 79446.

The probes having the nucleotide sequence represented by SEQ ID NOS: 128708, 128709, 128710, 128711, 128712, 128713, 128714, 128715, 128716, 128717, 128718 were selected from the ORFs having the nucleotide sequence represented by SEQ ID NO: 79447.

The probes having the nucleotide sequence represented by SEQ ID NOS: 128719, 128720, 128721, 128722, 128723, 128724, 128725, 128726, 128727, 128728, 128729 were selected from the ORFs having the nucleotide sequence represented by SEQ ID NO: 79448.

The probes having the nucleotide sequence represented by SEQ ID NOS: 128730, 128731, 128732, 128733, 128734, 128735, 128736, 128737, 128738, 128739, 128740 were selected from the ORFs having the nucleotide sequence represented by SEQ ID NO: 79449.

The probes having the nucleotide sequence represented by SEQ ID NOS: 128741, 128742, 128743, 128744, 128745, 128746, 128747, 128748, 128749, 128750, 128751, 128752, 128753, 128754, 128755, 128756, 128757, 128758, 128759, 128760, 128761, 128762 were selected from the ORFs having the nucleotide sequence represented by SEQ ID NO: 79450.

The probes having the nucleotide sequence represented by SEQ ID NOS: 128763, 128764, 128765, 128766, 128767, 128768, 128769, 128770, 128771, 128772, 128773 were selected from the ORFs having the nucleotide sequence represented by SEQ ID NO: 79451.

The probes having the nucleotide sequence represented by SEQ ID NOS: 128774, 128775, 128776, 128777, 128778, 128779, 128780, 128781, 128782, 128783, 128784 were selected from the ORFs having the nucleotide sequence represented by SEQ ID NO: 79452.

The probes having the nucleotide sequence represented by SEQ ID NOS: 128785, 128786, 128787, 128788, 128789, 128790, 128791, 128792, 128793, 128794, 128795 were selected from the ORFs having the nucleotide sequence represented by SEQ ID NO: 79453.

The probes having the nucleotide sequence represented by SEQ ID NOS: 128796, 128797, 128798, 128799, 128800, 128801, 128802, 128803, 128804, 128805, 128806 were selected from the ORFs having the nucleotide sequence represented by SEQ ID NO: 79454.

The probes having the nucleotide sequence represented by SEQ ID NOS: 128807, 128808, 128809, 128810, 128811, 128812, 128813, 128814, 128815, 128816, 128817 were selected from the ORFs having the nucleotide sequence represented by SEQ ID NO: 79455.

The probes having the nucleotide sequence represented by SEQ ID NOS: 128818, 128819, 128820, 128821, 128822, 128823, 128824, 128825, 128826, 128827, 128828 were selected from the ORFs having the nucleotide sequence represented by SEQ ID NO: 79456.

The probes having the nucleotide sequence represented by SEQ ID NOS: 128829, 128830, 128831, 128832, 128833, 128834, 128835, 128836, 128837, 128838, 128839 were selected from the ORFs having the nucleotide sequence represented by SEQ ID NO: 79457.

The probes having the nucleotide sequence represented by SEQ ID NOS: 128840, 128841, 128842, 128843, 128844, 128845, 128846, 128847, 128848, 128849, 128850 were selected from the ORFs having the nucleotide sequence represented by SEQ ID NO: 79458.

The probes having the nucleotide sequence represented by SEQ ID NOS: 128851, 128852, 128853, 128854, 128855, 128856, 128857, 128858, 128859, 128860, 128861 were selected from the ORFs having the nucleotide sequence represented by SEQ ID NO: 79459.

The probes having the nucleotide sequence represented by SEQ ID NOS: 128862, 128863, 128864, 128865, 128866, 128867, 128868, 128869, 128870, 128871, 128872 were selected from the ORFs having the nucleotide sequence represented by SEQ ID NO: 79460.

The probes having the nucleotide sequence represented by SEQ ID NOS: 128873, 128874, 128875, 128876, 128877, 128878, 128879, 128880, 128881, 128882, 128883 were selected from the ORFs having the nucleotide sequence represented by SEQ ID NO: 79461.

The probes having the nucleotide sequence represented by SEQ ID NOS: 128884, 128885, 128886, 128887, 128888, 128889, 128890, 128891, 128892, 128893, 128894 were selected from the ORFs having the nucleotide sequence represented by SEQ ID NO: 79462.

The probes having the nucleotide sequence represented by SEQ ID NOS: 128895, 128896, 128897, 128898, 128899, 128900, 128901, 128902, 128903, 128904, 128905 were selected from the ORFs having the nucleotide sequence represented by SEQ ID NO: 79463.

The probes having the nucleotide sequence represented by SEQ ID NOS: 128906, 128907, 128908, 128909, 128910, 128911, 128912, 128913, 128914, 128915, 128916 were selected from the ORFs having the nucleotide sequence represented by SEQ ID NO: 79464.

The probes having the nucleotide sequence represented by SEQ ID NOS: 128917, 128918, 128919, 128920, 128921, 128922, 128923, 128924, 128925, 128926, 128927 were selected from the ORFs having the nucleotide sequence represented by SEQ ID NO: 79465.

The probes having the nucleotide sequence represented by SEQ ID NOS: 128928, 128929, 128930, 128931, 128932, 128933, 128934, 128935, 128936, 128937, 128938 were selected from the ORFs having the nucleotide sequence represented by SEQ ID NO: 79466.

The probes having the nucleotide sequence represented by SEQ ID NOS: 128939, 128940, 128941, 128942, 128943, 128944, 128945, 128946, 128947, 128948, 128949 were selected from the ORFs having the nucleotide sequence represented by SEQ ID NO: 79467.

The probes having the nucleotide sequence represented by SEQ ID NOS: 128950, 128951, 128952, 128953, 128954, 128955, 128956, 128957, 128958, 128959, 128960 were selected from the ORFs having the nucleotide sequence represented by SEQ ID NO: 79468.

The probes having the nucleotide sequence represented by SEQ ID NOS: 128961, 128962, 128963, 128964, 128965, 128966, 128967, 128968, 128969, 128970, 128971 were selected from the ORFs having the nucleotide sequence represented by SEQ ID NO: 79469.

The probes having the nucleotide sequence represented by SEQ ID NOS: 128972, 128973, 128974, 128975, 128976, 128977, 128978, 128979, 128980, 128981, 128982 were selected from the ORFs having the nucleotide sequence represented by SEQ ID NO: 79470.

The probes having the nucleotide sequence represented by SEQ ID NOS: 128983, 128984, 128985, 128986, 128987, 128988, 128989, 128990, 128991, 128992, 128993 were selected from the ORFs having the nucleotide sequence represented by SEQ ID NO: 79471.

The probes having the nucleotide sequence represented by SEQ ID NOS: 128994, 128995, 128996, 128997, 128998, 128999, 129000, 129001, 129002, 129003, 129004 were selected from the ORFs having the nucleotide sequence represented by SEQ ID NO: 79472.

The probes having the nucleotide sequence represented by SEQ ID NOS: 129005, 129006, 129007, 129008, 129009, 129010, 129011, 129012, 129013, 129014, 129015 were selected from the ORFs having the nucleotide sequence represented by SEQ ID NO: 79473.

The probes having the nucleotide sequence represented by SEQ ID NOS: 129016, 129017, 129018, 129019, 129020, 129021, 129022, 129023, 129024, 129025, 129026 were selected from the ORFs having the nucleotide sequence represented by SEQ ID NO: 79474.

The probes having the nucleotide sequence represented by SEQ ID NOS: 129027, 129028, 129029, 129030, 129031, 129032, 129033, 129034, 129035, 129036, 129037 were selected from the ORFs having the nucleotide sequence represented by SEQ ID NO: 79475.

The probes having the nucleotide sequence represented by SEQ ID NOS: 129038, 129039, 129040, 129041, 129042, 129043, 129044, 129045, 129046, 129047, 129048 were selected from the ORFs having the nucleotide sequence represented by SEQ ID NO: 79476.

The probes having the nucleotide sequence represented by SEQ ID NOS: 129049, 129050, 129051, 129052, 129053, 129054, 129055, 129056, 129057, 129058, 129059 were selected from the ORFs having the nucleotide sequence represented by SEQ ID NO: 79477.

The probes having the nucleotide sequence represented by SEQ ID NOS: 129060, 129061, 129062, 129063, 129064, 129065, 129066, 129067, 129068, 129069, 129070, 129071, 129072, 129073, 129074, 129075, 129076, 129077, 129078, 129079, 129080, 129081 were selected from the ORFs having the nucleotide sequence represented by SEQ ID NO: 79478.

The probes having the nucleotide sequence represented by SEQ ID NOS: 129082, 129083, 129084, 129085, 129086, 129087, 129088, 129089, 129090, 129091, 129092 were selected from the ORFs having the nucleotide sequence represented by SEQ ID NO: 79479.

The probes having the nucleotide sequence represented by SEQ ID NOS: 129093, 129094, 129095, 129096, 129097, 129098, 129099, 129100, 129101, 129102, 129103 were selected from the ORFs having the nucleotide sequence represented by SEQ ID NO: 79480.

The probes having the nucleotide sequence represented by SEQ ID NOS: 129104, 129105, 129106, 129107, 129108, 129109, 129110, 129111, 129112, 129113, 129114 were selected from the ORFs having the nucleotide sequence represented by SEQ ID NO: 79481.

The probes having the nucleotide sequence represented by SEQ ID NOS: 129115, 129116, 129117, 129118, 129119, 129120, 129121, 129122, 129123, 129124, 129125 were selected from the ORFs having the nucleotide sequence represented by SEQ ID NO: 79482.

The probes having the nucleotide sequence represented by SEQ ID NOS: 129126, 129127, 129128, 129129, 129130, 129131, 129132, 129133, 129134, 129135, 129136 were selected from the ORFs having the nucleotide sequence represented by SEQ ID NO: 79483.

The probes having the nucleotide sequence represented by SEQ ID NOS: 129137, 129138, 129139, 129140, 129141, 129142, 129143, 129144, 129145, 129146, 129147 were selected from the ORFs having the nucleotide sequence represented by SEQ ID NO: 79484.

The probes having the nucleotide sequence represented by SEQ ID NOS: 129148, 129149, 129150, 129151, 129152, 129153, 129154, 129155, 129156, 129157, 129158 were selected from the ORFs having the nucleotide sequence represented by SEQ ID NO: 79485.

The probes having the nucleotide sequence represented by SEQ ID NOS: 129159, 129160, 129161, 129162, 129163, 129164, 129165, 129166, 129167, 129168, 129169 were selected from the ORFs having the nucleotide sequence represented by SEQ ID NO: 79486.

The probes having the nucleotide sequence represented by SEQ ID NOS: 129170, 129171, 129172, 129173, 129174, 129175, 129176, 129177, 129178, 129179, 129180 were selected from the ORFs having the nucleotide sequence represented by SEQ ID NO: 79487.

The probes having the nucleotide sequence represented by SEQ ID NOS: 129181, 129182, 129183, 129184, 129185, 129186, 129187, 129188, 129189, 129190, 129191 were selected from the ORFs having the nucleotide sequence represented by SEQ ID NO: 79488.

The probes having the nucleotide sequence represented by SEQ ID NOS: 129192, 129193, 129194, 129195, 129196, 129197, 129198, 129199, 129200, 129201, 129202 were selected from the ORFs having the nucleotide sequence represented by SEQ ID NO: 79489.

The probes having the nucleotide sequence represented by SEQ ID NOS: 129203, 129204, 129205, 129206, 129207, 129208, 129209, 129210, 129211, 129212, 129213 were selected from the ORFs having the nucleotide sequence represented by SEQ ID NO: 79490.

The probes having the nucleotide sequence represented by SEQ ID NOS: 129214, 129215, 129216, 129217, 129218, 129219, 129220, 129221, 129222, 129223, 129224 were selected from the ORFs having the nucleotide sequence represented by SEQ ID NO: 79491.

The probes having the nucleotide sequence represented by SEQ ID NOS: 129225, 129226, 129227, 129228, 129229, 129230, 129231, 129232, 129233, 129234, 129235 were selected from the ORFs having the nucleotide sequence represented by SEQ ID NO: 79492.

The probes having the nucleotide sequence represented by SEQ ID NOS: 129236, 129237, 129238, 129239, 129240, 129241, 129242, 129243, 129244, 129245, 129246 were selected from the ORFs having the nucleotide sequence represented by SEQ ID NO: 79493.

The probes having the nucleotide sequence represented by SEQ ID NOS: 129247, 129248, 129249, 129250, 129251, 129252, 129253, 129254, 129255, 129256, 129257 were selected from the ORFs having the nucleotide sequence represented by SEQ ID NO: 79494.

The probes having the nucleotide sequence represented by SEQ ID NOS: 129258, 129259, 129260, 129261, 129262, 129263, 129264, 129265, 129266, 129267, 129268 were selected from the ORFs having the nucleotide sequence represented by SEQ ID NO: 79495.

The probes having the nucleotide sequence represented by SEQ ID NOS: 129269, 129270, 129271, 129272, 129273, 129274, 129275, 129276, 129277, 129278, 129279 were selected from the ORFs having the nucleotide sequence represented by SEQ ID NO: 79496.

The probes having the nucleotide sequence represented by SEQ ID NOS: 129280, 129281, 129282, 129283, 129284, 129285, 129286, 129287, 129288, 129289, 129290 were selected from the ORFs having the nucleotide sequence represented by SEQ ID NO: 79497.

The probes having the nucleotide sequence represented by SEQ ID NOS: 129291, 129292, 129293, 129294, 129295, 129296, 129297, 129298, 129299, 129300, 129301 were selected from the ORFs having the nucleotide sequence represented by SEQ ID NO: 79498.

The probes having the nucleotide sequence represented by SEQ ID NOS: 129302, 129303, 129304, 129305, 129306, 129307, 129308, 129309, 129310, 129311, 129312 were selected from the ORFs having the nucleotide sequence represented by SEQ ID NO: 79499.

The probes having the nucleotide sequence represented by SEQ ID NOS: 129313, 129314, 129315, 129316, 129317, 129318, 129319, 129320, 129321, 129322, 129323 were selected from the ORFs having the nucleotide sequence represented by SEQ ID NO: 79500.

The probes having the nucleotide sequence represented by SEQ ID NOS: 129324, 129325, 129326, 129327, 129328, 129329, 129330, 129331, 129332, 129333, 129334 were selected from the ORFs having the nucleotide sequence represented by SEQ ID NO: 79501.

The probes having the nucleotide sequence represented by SEQ ID NOS: 129335, 129336, 129337, 129338, 129339, 129340, 129341, 129342, 129343, 129344, 129345 were selected from the ORFs having the nucleotide sequence represented by SEQ ID NO: 79502.

The probes having the nucleotide sequence represented by SEQ ID NOS: 129346, 129347, 129348, 129349, 129350, 129351, 129352, 129353, 129354, 129355, 129356 were selected from the ORFs having the nucleotide sequence represented by SEQ ID NO: 79503.

The probes having the nucleotide sequence represented by SEQ ID NOS: 129357, 129358, 129359, 129360, 129361, 129362, 129363, 129364, 129365, 129366, 129367 were selected from the ORFs having the nucleotide sequence represented by SEQ ID NO: 79504.

The probes having the nucleotide sequence represented by SEQ ID NOS: 129368, 129369, 129370, 129371, 129372, 129373, 129374, 129375, 129376, 129377, 129378 were selected from the ORFs having the nucleotide sequence represented by SEQ ID NO: 79505.

The probes having the nucleotide sequence represented by SEQ ID NOS: 129379, 129380, 129381, 129382, 129383, 129384, 129385, 129386, 129387, 129388, 129389 were selected from the ORFs having the nucleotide sequence represented by SEQ ID NO: 79506.

The probes having the nucleotide sequence represented by SEQ ID NOS: 129390, 129391, 129392, 129393, 129394, 129395, 129396, 129397, 129398, 129399, 129400 were selected from the ORFs having the nucleotide sequence represented by SEQ ID NO: 79507.

The probes having the nucleotide sequence represented by SEQ ID NOS: 129401, 129402, 129403, 129404, 129405, 129406, 129407, 129408, 129409, 129410, 129411 were selected from the ORFs having the nucleotide sequence represented by SEQ ID NO: 79508.

The probes having the nucleotide sequence represented by SEQ ID NOS: 129412, 129413, 129414, 129415, 129416, 129417, 129418, 129419, 129420, 129421, 129422 were selected from the ORFs having the nucleotide sequence represented by SEQ ID NO: 79509.

The probes having the nucleotide sequence represented by SEQ ID NOS: 129423, 129424, 129425, 129426, 129427, 129428, 129429, 129430, 129431, 129432, 129433 were selected from the ORFs having the nucleotide sequence represented by SEQ ID NO: 79510.

The probes having the nucleotide sequence represented by SEQ ID NOS: 129434, 129435, 129436, 129437, 129438, 129439, 129440, 129441, 129442, 129443, 129444 were selected from the ORFs having the nucleotide sequence represented by SEQ ID NO: 79511.

The probes having the nucleotide sequence represented by SEQ ID NOS: 129445, 129446, 129447, 129448, 129449, 129450, 129451, 129452, 129453, 129454, 129455 were selected from the ORFs having the nucleotide sequence represented by SEQ ID NO: 79512.

The probes having the nucleotide sequence represented by SEQ ID NOS: 129456, 129457, 129458, 129459, 129460, 129461, 129462, 129463, 129464, 129465, 129466 were selected from the ORFs having the nucleotide sequence represented by SEQ ID NO: 79513.

The probes having the nucleotide sequence represented by SEQ ID NOS: 129467, 129468, 129469, 129470, 129471, 129472, 129473, 129474, 129475, 129476, 129477 were selected from the ORFs having the nucleotide sequence represented by SEQ ID NO: 79514.

The probes having the nucleotide sequence represented by SEQ ID NOS: 129478, 129479, 129480, 129481, 129482, 129483, 129484, 129485, 129486, 129487, 129488 were selected from the ORFs having the nucleotide sequence represented by SEQ ID NO: 79515.

The probes having the nucleotide sequence represented by SEQ ID NOS: 129489, 129490, 129491, 129492, 129493, 129494, 129495, 129496, 129497, 129498, 129499 were selected from the ORFs having the nucleotide sequence represented by SEQ ID NO: 79516.

The probes having the nucleotide sequence represented by SEQ ID NOS: 129500, 129501, 129502, 129503, 129504, 129505, 129506, 129507, 129508, 129509, 129510 were selected from the ORFs having the nucleotide sequence represented by SEQ ID NO: 79517.

The probes having the nucleotide sequence represented by SEQ ID NOS: 129511, 129512, 129513, 129514, 129515, 129516, 129517, 129518, 129519, 129520, 129521 were selected from the ORFs having the nucleotide sequence represented by SEQ ID NO: 79518.

The probes having the nucleotide sequence represented by SEQ ID NOS: 129522, 129523, 129524, 129525, 129526, 129527, 129528, 129529, 129530, 129531, 129532 were selected from the ORFs having the nucleotide sequence represented by SEQ ID NO: 79519.

The probes having the nucleotide sequence represented by SEQ ID NOS: 129533, 129534, 129535, 129536, 129537, 129538, 129539, 129540, 129541, 129542, 129543 were selected from the ORFs having the nucleotide sequence represented by SEQ ID NO: 79520.

The probes having the nucleotide sequence represented by SEQ ID NOS: 129544, 129545, 129546, 129547, 129548, 129549, 129550, 129551, 129552, 129553, 129554 were selected from the ORFs having the nucleotide sequence represented by SEQ ID NO: 79521.

The probes having the nucleotide sequence represented by SEQ ID NOS: 129555, 129556, 129557, 129558, 129559, 129560, 129561, 129562, 129563, 129564, 129565 were selected from the ORFs having the nucleotide sequence represented by SEQ ID NO: 79522.

The probes having the nucleotide sequence represented by SEQ ID NOS: 129566, 129567, 129568, 129569, 129570, 129571, 129572, 129573, 129574, 129575, 129576 were selected from the ORFs having the nucleotide sequence represented by SEQ ID NO: 79523.

The probes having the nucleotide sequence represented by SEQ ID NOS: 129577, 129578, 129579, 129580, 129581, 129582, 129583, 129584, 129585, 129586, 129587 were selected from the ORFs having the nucleotide sequence represented by SEQ ID NO: 79524.

The probes having the nucleotide sequence represented by SEQ ID NOS: 129588, 129589, 129590, 129591, 129592, 129593, 129594, 129595, 129596, 129597, 129598 were selected from the ORFs having the nucleotide sequence represented by SEQ ID NO: 79525.

The probes having the nucleotide sequence represented by SEQ ID NOS: 129599, 129600, 129601, 129602, 129603, 129604, 129605, 129606, 129607, 129608, 129609 were selected from the ORFs having the nucleotide sequence represented by SEQ ID NO: 79526.

The probes having the nucleotide sequence represented by SEQ ID NOS: 129610, 129611, 129612, 129613, 129614, 129615, 129616, 129617, 129618, 129619, 129620 were selected from the ORFs having the nucleotide sequence represented by SEQ ID NO: 79527.

The probes having the nucleotide sequence represented by SEQ ID NOS: 129621, 129622, 129623, 129624, 129625, 129626, 129627, 129628, 129629, 129630, 129631, 129632, 129633, 129634, 129635, 129636, 129637, 129638, 129639, 129640, 129641, 129642 were selected from the ORFs having the nucleotide sequence represented by SEQ ID NO: 79528.

The probes having the nucleotide sequence represented by SEQ ID NOS: 129643, 129644, 129645, 129646, 129647, 129648, 129649, 129650, 129651, 129652, 129653 were selected from the ORFs having the nucleotide sequence represented by SEQ ID NO: 79529.

The probes having the nucleotide sequence represented by SEQ ID NOS: 129654, 129655, 129656, 129657, 129658, 129659, 129660, 129661, 129662, 129663, 129664, 129665, 129666, 129667, 129668, 129669, 129670, 129671, 129672, 129673, 129674, 129675 were selected from the ORFs having the nucleotide sequence represented by SEQ ID NO: 79530.

The probes having the nucleotide sequence represented by SEQ ID NOS: 129676, 129677, 129678, 129679, 129680, 129681, 129682, 129683, 129684, 129685, 129686 were selected from the ORFs having the nucleotide sequence represented by SEQ ID NO: 79531.

The probes having the nucleotide sequence represented by SEQ ID NOS: 129687, 129688, 129689, 129690, 129691, 129692, 129693, 129694, 129695, 129696, 129697 were selected from the ORFs having the nucleotide sequence represented by SEQ ID NO: 79532.

The probes having the nucleotide sequence represented by SEQ ID NOS: 129698, 129699, 129700, 129701, 129702, 129703, 129704, 129705, 129706, 129707, 129708 were selected from the ORFs having the nucleotide sequence represented by SEQ ID NO: 79533.

The probes having the nucleotide sequence represented by SEQ ID NOS: 129709, 129710, 129711, 129712, 129713, 129714, 129715, 129716, 129717, 129718, 129719 were selected from the ORFs having the nucleotide sequence represented by SEQ ID NO: 79534.

The probes having the nucleotide sequence represented by SEQ ID NOS: 129720, 129721, 129722, 129723, 129724, 129725, 129726, 129727, 129728, 129729, 129730 were selected from the ORFs having the nucleotide sequence represented by SEQ ID NO: 79535.

The probes having the nucleotide sequence represented by SEQ ID NOS: 129731, 129732, 129733, 129734, 129735, 129736, 129737, 129738, 129739, 129740, 129741 were selected from the ORFs having the nucleotide sequence represented by SEQ ID NO: 79536.

The probes having the nucleotide sequence represented by SEQ ID NOS: 129742, 129743, 129744, 129745, 129746, 129747, 129748, 129749, 129750, 129751, 129752 were selected from the ORFs having the nucleotide sequence represented by SEQ ID NO: 79537.

The probes having the nucleotide sequence represented by SEQ ID NOS: 129753, 129754, 129755, 129756, 129757, 129758, 129759, 129760, 129761, 129762, 129763 were selected from the ORFs having the nucleotide sequence represented by SEQ ID NO: 79538.

The probes having the nucleotide sequence represented by SEQ ID NOS: 129764, 129765, 129766, 129767, 129768, 129769, 129770, 129771, 129772, 129773, 129774 were selected from the ORFs having the nucleotide sequence represented by SEQ ID NO: 79539.

The probes having the nucleotide sequence represented by SEQ ID NOS: 129775, 129776, 129777, 129778, 129779, 129780, 129781, 129782, 129783, 129784, 129785 were selected from the ORFs having the nucleotide sequence represented by SEQ ID NO: 79540.

The probes having the nucleotide sequence represented by SEQ ID NOS: 129786, 129787, 129788, 129789, 129790, 129791, 129792, 129793, 129794, 129795, 129796 were selected from the ORFs having the nucleotide sequence represented by SEQ ID NO: 79541.

The probes having the nucleotide sequence represented by SEQ ID NOS: 129797, 129798, 129799, 129800, 129801, 129802, 129803, 129804, 129805, 129806, 129807 were selected from the ORFs having the nucleotide sequence represented by SEQ ID NO: 79542.

The probes having the nucleotide sequence represented by SEQ ID NOS: 129808, 129809, 129810, 129811, 129812, 129813, 129814, 129815, 129816, 129817, 129818 were selected from the ORFs having the nucleotide sequence represented by SEQ ID NO: 79543.

The probes having the nucleotide sequence represented by SEQ ID NOS: 129819, 129820, 129821, 129822, 129823, 129824, 129825, 129826, 129827, 129828, 129829 were selected from the ORFs having the nucleotide sequence represented by SEQ ID NO: 79544.

The probes having the nucleotide sequence represented by SEQ ID NOS: 129830, 129831, 129832, 129833, 129834, 129835, 129836, 129837, 129838, 129839, 129840 were selected from the ORFs having the nucleotide sequence represented by SEQ ID NO: 79545.

The probes having the nucleotide sequence represented by SEQ ID NOS: 129841, 129842, 129843, 129844, 129845, 129846, 129847, 129848, 129849, 129850, 129851 were selected from the ORFs having the nucleotide sequence represented by SEQ ID NO: 79546.

The probes having the nucleotide sequence represented by SEQ ID NOS: 129852, 129853, 129854, 129855, 129856, 129857, 129858, 129859, 129860, 129861, 129862 were selected from the ORFs having the nucleotide sequence represented by SEQ ID NO: 79547.

The probes having the nucleotide sequence represented by SEQ ID NOS: 129863, 129864, 129865, 129866, 129867, 129868, 129869, 129870, 129871, 129872, 129873 were selected from the ORFs having the nucleotide sequence represented by SEQ ID NO: 79548.

The probes having the nucleotide sequence represented by SEQ ID NOS: 129874, 129875, 129876, 129877, 129878, 129879, 129880, 129881, 129882, 129883, 129884 were selected from the ORFs having the nucleotide sequence represented by SEQ ID NO: 79549.

The probes having the nucleotide sequence represented by SEQ ID NOS: 129885, 129886, 129887, 129888, 129889, 129890, 129891, 129892, 129893, 129894, 129895 were selected from the ORFs having the nucleotide sequence represented by SEQ ID NO: 79550.

The probes having the nucleotide sequence represented by SEQ ID NOS: 129896, 129897, 129898, 129899, 129900, 129901, 129902, 129903, 129904, 129905, 129906 were selected from the ORFs having the nucleotide sequence represented by SEQ ID NO: 79551.

The probes having the nucleotide sequence represented by SEQ ID NOS: 129907, 129908, 129909, 129910, 129911, 129912, 129913, 129914, 129915, 129916, 129917 were selected from

The ORFs having the nucleotide sequence represented by SEQ ID NO: 79552.

The probes having the nucleotide sequence represented by SEQ ID NOS: 129918, 129919, 129920, 129921, 129922, 129923, 129924, 129925, 129926, 129927, 129928 were selected from the ORFs having the nucleotide sequence represented by SEQ ID NO: 79553.

The probes having the nucleotide sequence represented by SEQ ID NOS: 129929, 129930, 129931, 129932, 129933, 129934, 129935, 129936, 129937, 129938, 129939 were selected from the ORFs having the nucleotide sequence represented by SEQ ID NO: 79554.

The probes having the nucleotide sequence represented by SEQ ID NOS: 129940, 129941, 129942, 129943, 129944, 129945, 129946, 129947, 129948, 129949, 129950 were selected from the ORFs having the nucleotide sequence represented by SEQ ID NO: 79555.

The probes having the nucleotide sequence represented by SEQ ID NOS: 129951, 129952, 129953, 129954, 129955, 129956, 129957, 129958, 129959, 129960, 129961 were selected from the ORFs having the nucleotide sequence represented by SEQ ID NO: 79556.

The probes having the nucleotide sequence represented by SEQ ID NOS: 129962, 129963, 129964, 129965, 129966, 129967, 129968, 129969, 129970, 129971, 129972 were selected from the ORFs having the nucleotide sequence represented by SEQ ID NO: 79557.

The probes having the nucleotide sequence represented by SEQ ID NOS: 129973, 129974, 129975, 129976, 129977, 129978, 129979, 129980, 129981, 129982, 129983 were selected from the ORFs having the nucleotide sequence represented by SEQ ID NO: 79558.

The probes having the nucleotide sequence represented by SEQ ID NOS: 129984, 129985, 129986, 129987, 129988, 129989, 129990, 129991, 129992, 129993, 129994 were selected from the ORFs having the nucleotide sequence represented by SEQ ID NO: 79559.

The probes having the nucleotide sequence represented by SEQ ID NOS: 129995, 129996, 129997, 129998, 129999, 130000, 130001, 130002, 130003, 130004, 130005 were selected from the ORFs having the nucleotide sequence represented by SEQ ID NO: 79560.

The probes having the nucleotide sequence represented by SEQ ID NOS: 130006, 130007, 130008, 130009, 130010, 130011, 130012, 130013, 130014, 130015, 130016 were selected from the ORFs having the nucleotide sequence represented by SEQ ID NO: 79561.

The probes having the nucleotide sequence represented by SEQ ID NOS: 130017, 130018, 130019, 130020, 130021, 130022, 130023, 130024, 130025, 130026, 130027 were selected from the ORFs having the nucleotide sequence represented by SEQ ID NO: 79562.

The probes having the nucleotide sequence represented by SEQ ID NOS: 130028, 130029, 130030, 130031, 130032, 130033, 130034, 130035, 130036, 130037, 130038 were selected from the ORFs having the nucleotide sequence represented by SEQ ID NO: 79563.

The probes having the nucleotide sequence represented by SEQ ID NOS: 130039, 130040, 130041, 130042, 130043, 130044, 130045, 130046, 130047, 130048, 130049 were selected from the ORFs having the nucleotide sequence represented by SEQ ID NO: 79564.

The probes having the nucleotide sequence represented by SEQ ID NOS: 130050, 130051, 130052, 130053, 130054, 130055, 130056, 130057, 130058, 130059, 130060 were selected from the ORFs having the nucleotide sequence represented by SEQ ID NO: 79565.

The probes having the nucleotide sequence represented by SEQ ID NOS: 130061, 130062, 130063, 130064, 130065, 130066, 130067, 130068, 130069, 130070, 130071 were selected from the ORFs having the nucleotide sequence represented by SEQ ID NO: 79566.

The probes having the nucleotide sequence represented by SEQ ID NOS: 130072, 130073, 130074, 130075, 130076, 130077, 130078, 130079, 130080, 130081, 130082 were selected from the ORFs having the nucleotide sequence represented by SEQ ID NO: 79567.

The probes having the nucleotide sequence represented by SEQ ID NOS: 130083, 130084, 130085, 130086, 130087, 130088, 130089, 130090, 130091, 130092, 130093 were selected from the ORFs having the nucleotide sequence represented by SEQ ID NO: 79568.

The probes having the nucleotide sequence represented by SEQ ID NOS: 130094, 130095, 130096, 130097, 130098, 130099, 130100, 130101, 130102, 130103, 130104 were selected from the ORFs having the nucleotide sequence represented by SEQ ID NO: 79569.

The probes having the nucleotide sequence represented by SEQ ID NOS: 130105, 130106, 130107, 130108, 130109, 130110, 130111, 130112, 130113, 130114, 130115 were selected from the ORFs having the nucleotide sequence represented by SEQ ID NO: 79570.

The probes having the nucleotide sequence represented by SEQ ID NOS: 130116, 130117, 130118, 130119, 130120, 130121, 130122, 130123, 130124, 130125, 130126 were selected from the ORFs having the nucleotide sequence represented by SEQ ID NO: 79571.

The probes having the nucleotide sequence represented by SEQ ID NOS: 130127, 130128, 130129, 130130, 130131, 130132, 130133, 130134, 130135, 130136, 130137 were selected from the ORFs having the nucleotide sequence represented by SEQ ID NO: 79572.

The probes having the nucleotide sequence represented by SEQ ID NOS: 130138, 130139, 130140, 130141, 130142, 130143, 130144, 130145, 130146, 130147, 130148 were selected from the ORFs having the nucleotide sequence represented by SEQ ID NO: 79573.

The probes having the nucleotide sequence represented by SEQ ID NOS: 130149, 130150, 130151, 130152, 130153, 130154, 130155, 130156, 130157, 130158, 130159 were selected from the ORFs having the nucleotide sequence represented by SEQ ID NO: 79574.

The probes having the nucleotide sequence represented by SEQ ID NOS: 130160, 130161, 130162, 130163, 130164, 130165, 130166, 130167, 130168, 130169, 130170 were selected from the ORFs having the nucleotide sequence represented by SEQ ID NO: 79575.

The probes having the nucleotide sequence represented by SEQ ID NOS: 130171, 130172, 130173, 130174, 130175, 130176, 130177, 130178, 130179, 130180, 130181 were selected from the ORFs having the nucleotide sequence represented by SEQ ID NO: 79576.

The probes having the nucleotide sequence represented by SEQ ID NOS: 130182, 130183, 130184, 130185, 130186, 130187, 130188, 130189, 130190, 130191, 130192 were selected from the ORFs having the nucleotide sequence represented by SEQ ID NO: 79577.

The probes having the nucleotide sequence represented by SEQ ID NOS: 130193, 130194, 130195, 130196, 130197, 130198, 130199, 130200, 130201, 130202, 130203 were selected from the ORFs having the nucleotide sequence represented by SEQ ID NO: 79578.

The probes having the nucleotide sequence represented by SEQ ID NOS: 130204, 130205, 130206, 130207, 130208, 130209, 130210, 130211, 130212, 130213, 130214 were selected from the ORFs having the nucleotide sequence represented by SEQ ID NO: 79579.

The probes having the nucleotide sequence represented by SEQ ID NOS: 130215, 130216, 130217, 130218, 130219, 130220, 130221, 130222, 130223, 130224, 130225 were selected from the ORFs having the nucleotide sequence represented by SEQ ID NO: 79580.

The probes having the nucleotide sequence represented by SEQ ID NOS: 130226, 130227, 130228, 130229, 130230, 130231, 130232, 130233, 130234, 130235, 130236 were selected from the ORFs having the nucleotide sequence represented by SEQ ID NO: 79581.

The probes having the nucleotide sequence represented by SEQ ID NOS: 130237, 130238, 130239, 130240, 130241, 130242, 130243, 130244, 130245, 130246, 130247 were selected from the ORFs having the nucleotide sequence represented by SEQ ID NO: 79582.

The probes having the nucleotide sequence represented by SEQ ID NOS: 130248, 130249, 130250, 130251, 130252, 130253, 130254, 130255, 130256, 130257, 130258 were selected from the ORFs having the nucleotide sequence represented by SEQ ID NO: 79583.

The probes having the nucleotide sequence represented by SEQ ID NOS: 130259, 130260, 130261, 130262, 130263, 130264, 130265, 130266, 130267, 130268, 130269 were selected from the ORFs having the nucleotide sequence represented by SEQ ID NO: 79584.

The probes having the nucleotide sequence represented by SEQ ID NOS: 130270, 130271, 130272, 130273, 130274, 130275, 130276, 130277, 130278, 130279, 130280 were selected from the ORFs having the nucleotide sequence represented by SEQ ID NO: 79585.

The probes having the nucleotide sequence represented by SEQ ID NOS: 130281, 130282, 130283, 130284, 130285, 130286, 130287, 130288, 130289, 130290, 130291 were selected from the ORFs having the nucleotide sequence represented by SEQ ID NO: 79586.

The probes having the nucleotide sequence represented by SEQ ID NOS: 130292, 130293, 130294, 130295, 130296, 130297, 130298, 130299, 130300, 130301, 130302 were selected from the ORFs having the nucleotide sequence represented by SEQ ID NO: 79587.

The probes having the nucleotide sequence represented by SEQ ID NOS: 130303, 130304, 130305, 130306, 130307, 130308, 130309, 130310, 130311, 130312, 130313 were selected from the ORFs having the nucleotide sequence represented by SEQ ID NO: 79588.

The probes having the nucleotide sequence represented by SEQ ID NOS: 130314, 130315, 130316, 130317, 130318, 130319, 130320, 130321, 130322, 130323, 130324 were selected from the ORFs having the nucleotide sequence represented by SEQ ID NO: 79589.

The probes having the nucleotide sequence represented by SEQ ID NOS: 130325, 130326, 130327, 130328, 130329, 130330, 130331, 130332, 130333, 130334, 130335 were selected from the ORFs having the nucleotide sequence represented by SEQ ID NO: 79590.

The probes having the nucleotide sequence represented by SEQ ID NOS: 130336, 130337, 130338, 130339, 130340, 130341, 130342, 130343, 130344, 130345, 130346 were selected from the ORFs having the nucleotide sequence represented by SEQ ID NO: 79591.

The probes having the nucleotide sequence represented by SEQ ID NOS: 130347, 130348, 130349, 130350, 130351, 130352, 130353, 130354, 130355, 130356, 130357 were selected from the ORFs having the nucleotide sequence represented by SEQ ID NO: 79592.

The probes having the nucleotide sequence represented by SEQ ID NOS: 130358, 130359, 130360, 130361, 130362, 130363, 130364, 130365, 130366, 130367, 130368 were selected from the ORFs having the nucleotide sequence represented by SEQ ID NO: 79593.

The probes having the nucleotide sequence represented by SEQ ID NOS: 130369, 130370, 130371, 130372, 130373, 130374, 130375, 130376, 130377, 130378, 130379 were selected from the ORFs having the nucleotide sequence represented by SEQ ID NO: 79594.

The probes having the nucleotide sequence represented by SEQ ID NOS: 130380, 130381, 130382, 130383, 130384, 130385, 130386, 130387, 130388, 130389, 130390 were selected from the ORFs having the nucleotide sequence represented by SEQ ID NO: 79595.

The probes having the nucleotide sequence represented by SEQ ID NOS: 130391, 130392, 130393, 130394, 130395, 130396, 130397, 130398, 130399, 130400, 130401 were selected from the ORFs having the nucleotide sequence represented by SEQ ID NO: 79596.

The probes having the nucleotide sequence represented by SEQ ID NOS: 130402, 130403, 130404, 130405, 130406, 130407, 130408, 130409, 130410, 130411, 130412 were selected from the ORFs having the nucleotide sequence represented by SEQ ID NO: 79597.

The probes having the nucleotide sequence represented by SEQ ID NOS: 130413, 130414, 130415, 130416, 130417, 130418, 130419, 130420, 130421, 130422, 130423 were selected from the ORFs having the nucleotide sequence represented by SEQ ID NO: 79598.

The probes having the nucleotide sequence represented by SEQ ID NOS: 130424, 130425, 130426, 130427, 130428, 130429, 130430, 130431, 130432, 130433, 130434 were selected from the ORFs having the nucleotide sequence represented by SEQ ID NO: 79599.

The probes having the nucleotide sequence represented by SEQ ID NOS: 130435, 130436, 130437, 130438, 130439, 130440, 130441, 130442, 130443, 130444, 130445 were selected from the ORFs having the nucleotide sequence represented by SEQ ID NO: 79600.

The probes having the nucleotide sequence represented by SEQ ID NOS: 130446, 130447, 130448, 130449, 130450, 130451, 130452, 130453, 130454, 130455, 130456 were selected from the ORFs having the nucleotide sequence represented by SEQ ID NO: 79601.

The probes having the nucleotide sequence represented by SEQ ID NOS: 130457, 130458, 130459, 130460, 130461, 130462, 130463, 130464, 130465, 130466, 130467 were selected from the ORFs having the nucleotide sequence represented by SEQ ID NO: 79602.

The probes having the nucleotide sequence represented by SEQ ID NOS: 130468, 130469, 130470, 130471, 130472, 130473, 130474, 130475, 130476, 130477, 130478 were selected from the ORFs having the nucleotide sequence represented by SEQ ID NO: 79603.

The probes having the nucleotide sequence represented by SEQ ID NOS: 130479, 130480, 130481, 130482, 130483, 130484, 130485, 130486, 130487, 130488, 130489 were selected from the ORFs having the nucleotide sequence represented by SEQ ID NO: 79604.

The probes having the nucleotide sequence represented by SEQ ID NOS: 130490, 130491, 130492, 130493, 130494, 130495, 130496, 130497, 130498, 130499, 130500 were selected from the ORFs having the nucleotide sequence represented by SEQ ID NO: 79605.

The probes having the nucleotide sequence represented by SEQ ID NOS: 130501, 130502, 130503, 130504, 130505, 130506, 130507, 130508, 130509, 130510, 130511 were selected from the ORFs having the nucleotide sequence represented by SEQ ID NO: 79606.

The probes having the nucleotide sequence represented by SEQ ID NOS: 130512, 130513, 130514, 130515, 130516, 130517, 130518, 130519, 130520, 130521, 130522 were selected from the ORFs having the nucleotide sequence represented by SEQ ID NO: 79607.

The probes having the nucleotide sequence represented by SEQ ID NOS: 130523, 130524, 130525, 130526, 130527, 130528, 130529, 130530, 130531, 130532, 130533 were selected from the ORFs having the nucleotide sequence represented by SEQ ID NO: 79608.

The probes having the nucleotide sequence represented by SEQ ID NOS: 130534, 130535, 130536, 130537, 130538, 130539, 130540, 130541, 130542, 130543, 130544 were selected from the ORFs having the nucleotide sequence represented by SEQ ID NO: 79609.

The probes having the nucleotide sequence represented by SEQ ID NOS: 130545, 130546, 130547, 130548, 130549, 130550, 130551, 130552, 130553, 130554, 130555 were selected from the ORFs having the nucleotide sequence represented by SEQ ID NO: 79610.

The probes having the nucleotide sequence represented by SEQ ID NOS: 130556, 130557, 130558, 130559, 130560, 130561, 130562, 130563, 130564, 130565, 130566 were selected from the ORFs having the nucleotide sequence represented by SEQ ID NO: 79611.

The probes having the nucleotide sequence represented by SEQ ID NOS: 130567, 130568, 130569, 130570, 130571, 130572, 130573, 130574, 130575, 130576, 130577 were selected from the ORFs having the nucleotide sequence represented by SEQ ID NO: 79612.

The probes having the nucleotide sequence represented by SEQ ID NOS: 130578, 130579, 130580, 130581, 130582, 130583, 130584, 130585, 130586, 130587, 130588 were selected from the ORFs having the nucleotide sequence represented by SEQ ID NO: 79613.

The probes having the nucleotide sequence represented by SEQ ID NOS: 130589, 130590, 130591, 130592, 130593, 130594, 130595, 130596, 130597, 130598, 130599 were selected from the ORFs having the nucleotide sequence represented by SEQ ID NO: 79614.

The probes having the nucleotide sequence represented by SEQ ID NOS: 130600, 130601, 130602, 130603, 130604, 130605, 130606, 130607, 130608, 130609, 130610 were selected from the ORFs having the nucleotide sequence represented by SEQ ID NO: 79615.

The probes having the nucleotide sequence represented by SEQ ID NOS: 130611, 130612, 130613, 130614, 130615, 130616, 130617, 130618, 130619, 130620, 130621 were selected from the ORFs having the nucleotide sequence represented by SEQ ID NO: 79616.

The probes having the nucleotide sequence represented by SEQ ID NOS: 130622, 130623, 130624, 130625, 130626, 130627, 130628, 130629, 130630, 130631, 130632 were selected from the ORFs having the nucleotide sequence represented by SEQ ID NO: 79617.

The probes having the nucleotide sequence represented by SEQ ID NOS: 130633, 130634, 130635, 130636, 130637, 130638, 130639, 130640, 130641, 130642, 130643 were selected from the ORFs having the nucleotide sequence represented by SEQ ID NO: 79618.

The probes having the nucleotide sequence represented by SEQ ID NOS: 130644, 130645, 130646, 130647, 130648, 130649, 130650, 130651, 130652, 130653, 130654 were selected from the ORFs having the nucleotide sequence represented by SEQ ID NO: 79619.

The probes having the nucleotide sequence represented by SEQ ID NOS: 130655, 130656, 130657, 130658, 130659, 130660, 130661, 130662, 130663, 130664, 130665 were selected from the ORFs having the nucleotide sequence represented by SEQ ID NO: 79620.

The probes having the nucleotide sequence represented by SEQ ID NOS: 130666, 130667, 130668, 130669, 130670, 130671, 130672, 130673, 130674, 130675, 130676 were selected from the ORFs having the nucleotide sequence represented by SEQ ID NO: 79621.

The probes having the nucleotide sequence represented by SEQ ID NOS: 130677, 130678, 130679, 130680, 130681, 130682, 130683, 130684, 130685, 130686, 130687 were selected from the ORFs having the nucleotide sequence represented by SEQ ID NO: 79622.

The probes having the nucleotide sequence represented by SEQ ID NOS: 130688, 130689, 130690, 130691, 130692, 130693, 130694, 130695, 130696, 130697, 130698 were selected from the ORFs having the nucleotide sequence represented by SEQ ID NO: 79623.

The probes having the nucleotide sequence represented by SEQ ID NOS: 130699, 130700, 130701, 130702, 130703, 130704, 130705, 130706, 130707, 130708, 130709 were selected from the ORFs having the nucleotide sequence represented by SEQ ID NO: 79624.

The probes having the nucleotide sequence represented by SEQ ID NOS: 130710, 130711, 130712, 130713, 130714, 130715, 130716, 130717, 130718, 130719, 130720 were selected from the ORFs having the nucleotide sequence represented by SEQ ID NO: 79625.

The probes having the nucleotide sequence represented by SEQ ID NOS: 130721, 130722, 130723, 130724, 130725, 130726, 130727, 130728, 130729, 130730, 130731 were selected from the ORFs having the nucleotide sequence represented by SEQ ID NO: 79626.

The probes having the nucleotide sequence represented by SEQ ID NOS: 130732, 130733, 130734, 130735, 130736, 130737, 130738, 130739, 130740, 130741, 130742 were selected from the ORFs having the nucleotide sequence represented by SEQ ID NO: 79627.

The probes having the nucleotide sequence represented by SEQ ID NOS: 130743, 130744, 130745, 130746, 130747, 130748, 130749, 130750, 130751, 130752, 130753 were selected from the ORFs having the nucleotide sequence represented by SEQ ID NO: 79628.

The probes having the nucleotide sequence represented by SEQ ID NOS: 130754, 130755, 130756, 130757, 130758, 130759, 130760, 130761, 130762, 130763, 130764 were selected from the ORFs having the nucleotide sequence represented by SEQ ID NO: 79629.

The probes having the nucleotide sequence represented by SEQ ID NOS: 130765, 130766, 130767, 130768, 130769, 130770, 130771, 130772, 130773, 130774, 130775 were selected from the ORFs having the nucleotide sequence represented by SEQ ID NO: 79630.

The probes having the nucleotide sequence represented by SEQ ID NOS: 130776, 130777, 130778, 130779, 130780, 130781, 130782, 130783, 130784, 130785, 130786 were selected from the ORFs having the nucleotide sequence represented by SEQ ID NO: 79631.

The probes having the nucleotide sequence represented by SEQ ID NOS: 130787, 130788, 130789, 130790, 130791, 130792, 130793, 130794, 130795, 130796, 130797 were selected from the ORFs having the nucleotide sequence represented by SEQ ID NO: 79632.

The probes having the nucleotide sequence represented by SEQ ID NOS: 130798, 130799, 130800, 130801, 130802, 130803, 130804, 130805, 130806, 130807, 130808 were selected from the ORFs having the nucleotide sequence represented by SEQ ID NO: 79633.

The probes having the nucleotide sequence represented by SEQ ID NOS: 130809, 130810, 130811, 130812, 130813, 130814, 130815, 130816, 130817, 130818, 130819 were selected from the ORFs having the nucleotide sequence represented by SEQ ID NO: 79634.

The probes having the nucleotide sequence represented by SEQ ID NOS: 130820, 130821, 130822, 130823, 130824, 130825, 130826, 130827, 130828, 130829, 130830 were selected from the ORFs having the nucleotide sequence represented by SEQ ID NO: 79635.

The probes having the nucleotide sequence represented by SEQ ID NOS: 130831, 130832, 130833, 130834, 130835, 130836, 130837, 130838, 130839, 130840, 130841 were selected from the ORFs having the nucleotide sequence represented by SEQ ID NO: 79636.

The probes having the nucleotide sequence represented by SEQ ID NOS: 130842, 130843, 130844, 130845, 130846, 130847, 130848, 130849, 130850, 130851, 130852 were selected from the ORFs having the nucleotide sequence represented by SEQ ID NO: 79637.

The probes having the nucleotide sequence represented by SEQ ID NOS: 130853, 130854, 130855, 130856, 130857, 130858, 130859, 130860, 130861, 130862, 130863 were selected from the ORFs having the nucleotide sequence represented by SEQ ID NO: 79638.

The probes having the nucleotide sequence represented by SEQ ID NOS: 130864, 130865, 130866, 130867, 130868, 130869, 130870, 130871, 130872, 130873, 130874 were selected from the ORFs having the nucleotide sequence represented by SEQ ID NO: 79639.

The probes having the nucleotide sequence represented by SEQ ID NOS: 130875, 130876, 130877, 130878, 130879, 130880, 130881, 130882, 130883, 130884, 130885 were selected from the ORFs having the nucleotide sequence represented by SEQ ID NO: 79640.

The probes having the nucleotide sequence represented by SEQ ID NOS: 130886, 130887, 130888, 130889, 130890, 130891, 130892, 130893, 130894, 130895, 130896 were selected from the ORFs having the nucleotide sequence represented by SEQ ID NO: 79641.

The probes having the nucleotide sequence represented by SEQ ID NOS: 130897, 130898, 130899, 130900, 130901, 130902, 130903, 130904, 130905, 130906, 130907 were selected from the ORFs having the nucleotide sequence represented by SEQ ID NO: 79642.

The probes having the nucleotide sequence represented by SEQ ID NOS: 130908, 130909, 130910, 130911, 130912, 130913, 130914, 130915, 130916, 130917, 130918 were selected from the ORFs having the nucleotide sequence represented by SEQ ID NO: 79643.

The probes having the nucleotide sequence represented by SEQ ID NOS: 130919, 130920, 130921, 130922, 130923, 130924, 130925, 130926, 130927, 130928, 130929 were selected from the ORFs having the nucleotide sequence represented by SEQ ID NO: 79644.

The probes having the nucleotide sequence represented by SEQ ID NOS: 130930, 130931, 130932, 130933, 130934, 130935, 130936, 130937, 130938, 130939, 130940 were selected from the ORFs having the nucleotide sequence represented by SEQ ID NO: 79645.

The probes having the nucleotide sequence represented by SEQ ID NOS: 130941, 130942, 130943, 130944, 130945, 130946, 130947, 130948, 130949, 130950, 130951 were selected from the ORFs having the nucleotide sequence represented by SEQ ID NO: 79646.

The probes having the nucleotide sequence represented by SEQ ID NOS: 130952, 130953, 130954, 130955, 130956, 130957, 130958, 130959, 130960, 130961, 130962 were selected from the ORFs having the nucleotide sequence represented by SEQ ID NO: 79647.

The probes having the nucleotide sequence represented by SEQ ID NOS: 130963, 130964, 130965, 130966, 130967, 130968, 130969, 130970, 130971, 130972, 130973 were selected from the ORFs having the nucleotide sequence represented by SEQ ID NO: 79648.

The probes having the nucleotide sequence represented by SEQ ID NOS: 130974, 130975, 130976, 130977, 130978, 130979, 130980, 130981, 130982, 130983, 130984 were selected from the ORFs having the nucleotide sequence represented by SEQ ID NO: 79649.

The probes having the nucleotide sequence represented by SEQ ID NOS: 130985, 130986, 130987, 130988, 130989, 130990, 130991, 130992, 130993, 130994, 130995 were selected from the ORFs having the nucleotide sequence represented by SEQ ID NO: 79650.

The probes having the nucleotide sequence represented by SEQ ID NOS: 130996, 130997, 130998, 130999, 131000, 131001, 131002, 131003, 131004, 131005, 131006 were selected from the ORFs having the nucleotide sequence represented by SEQ ID NO: 79651.

The probes having the nucleotide sequence represented by SEQ ID NOS: 131007, 131008, 131009, 131010, 131011, 131012, 131013, 131014, 131015, 131016, 131017 were selected from the ORFs having the nucleotide sequence represented by SEQ ID NO: 79652.

The probes having the nucleotide sequence represented by SEQ ID NOS: 131018, 131019, 131020, 131021, 131022, 131023, 131024, 131025, 131026, 131027, 131028 were selected from the ORFs having the nucleotide sequence represented by SEQ ID NO: 79653.

The probes having the nucleotide sequence represented by SEQ ID NOS: 131029, 131030, 131031, 131032, 131033, 131034, 131035, 131036, 131037, 131038, 131039 were selected from the ORFs having the nucleotide sequence represented by SEQ ID NO: 79654.

The probes having the nucleotide sequence represented by SEQ ID NOS: 131040, 131041, 131042, 131043, 131044, 131045, 131046, 131047, 131048, 131049, 131050 were selected from the ORFs having the nucleotide sequence represented by SEQ ID NO: 79655.

The probes having the nucleotide sequence represented by SEQ ID NOS: 131051, 131052, 131053, 131054, 131055, 131056, 131057, 131058, 131059, 131060, 131061 were selected from the ORFs having the nucleotide sequence represented by SEQ ID NO: 79656.

The probes having the nucleotide sequence represented by SEQ ID NOS: 131062, 131063, 131064, 131065, 131066, 131067, 131068, 131069, 131070, 131071, 131072 were selected from the ORFs having the nucleotide sequence represented by SEQ ID NO: 79657.

The probes having the nucleotide sequence represented by SEQ ID NOS: 131073, 131074, 131075, 131076, 131077, 131078, 131079, 131080, 131081, 131082, 131083, 131084, 131085, 131086, 131087, 131088, 131089, 131090, 131091, 131092, 131093, 131094 were selected from the ORFs having the nucleotide sequence represented by SEQ ID NO: 79658.

The probes having the nucleotide sequence represented by SEQ ID NOS: 131095, 131096, 131097, 131098, 131099, 131100, 131101, 131102, 131103, 131104, 131105, 131106, 131107, 131108, 131109, 131110, 131111, 131112, 131113, 131114, 131115, 131116 were selected from the ORFs having the nucleotide sequence represented by SEQ ID NO: 79659.

The probes having the nucleotide sequence represented by SEQ ID NOS: 131117, 131118, 131119, 131120, 131121, 131122, 131123, 131124, 131125, 131126, 131127 were selected from the ORFs having the nucleotide sequence represented by SEQ ID NO: 79660.

The probes having the nucleotide sequence represented by SEQ ID NOS: 131128, 131129, 131130, 131131, 131132, 131133, 131134, 131135, 131136, 131137, 131138 were selected from the ORFs having the nucleotide sequence represented by SEQ ID NO: 79661.

The probes having the nucleotide sequence represented by SEQ ID NOS: 131139, 131140, 131141, 131142, 131143, 131144, 131145, 131146, 131147, 131148, 131149 were selected from the ORFs having the nucleotide sequence represented by SEQ ID NO: 79662.

The probes having the nucleotide sequence represented by SEQ ID NOS: 131150, 131151, 131152, 131153, 131154, 131155, 131156, 131157, 131158, 131159, 131160 were selected from the ORFs having the nucleotide sequence represented by SEQ ID NO: 79663.

The probes having the nucleotide sequence represented by SEQ ID NOS: 131161, 131162, 131163, 131164, 131165, 131166, 131167, 131168, 131169, 131170, 131171 were selected from the ORFs having the nucleotide sequence represented by SEQ ID NO: 79664.

The probes having the nucleotide sequence represented by SEQ ID NOS: 131172, 131173, 131174, 131175, 131176, 131177, 131178, 131179, 131180, 131181, 131182 were selected from the ORFs having the nucleotide sequence represented by SEQ ID NO: 79665.

The probes having the nucleotide sequence represented by SEQ ID NOS: 131183, 131184, 131185, 131186, 131187, 131188, 131189, 131190, 131191, 131192, 131193 were selected from the ORFs having the nucleotide sequence represented by SEQ ID NO: 79666.

The probes having the nucleotide sequence represented by SEQ ID NOS: 131194, 131195, 131196, 131197, 131198, 131199, 131200, 131201, 131202, 131203, 131204 were selected from the ORFs having the nucleotide sequence represented by SEQ ID NO: 79667.

The probes having the nucleotide sequence represented by SEQ ID NOS: 131205, 131206, 131207, 131208, 131209, 131210, 131211, 131212, 131213, 131214, 131215 were selected from the ORFs having the nucleotide sequence represented by SEQ ID NO: 79668.

The probes having the nucleotide sequence represented by SEQ ID NOS: 131216, 131217, 131218, 131219, 131220, 131221, 131222, 131223, 131224, 131225, 131226 were selected from the ORFs having the nucleotide sequence represented by SEQ ID NO: 79669.

The probes having the nucleotide sequence represented by SEQ ID NOS: 131227, 131228, 131229, 131230, 131231, 131232, 131233, 131234, 131235, 131236, 131237 were selected from the ORFs having the nucleotide sequence represented by SEQ ID NO: 79670.

The probes having the nucleotide sequence represented by SEQ ID NOS: 131238, 131239, 131240, 131241, 131242, 131243, 131244, 131245, 131246, 131247, 131248 were selected from the ORFs having the nucleotide sequence represented by SEQ ID NO: 79671.

The probes having the nucleotide sequence represented by SEQ ID NOS: 131249, 131250, 131251, 131252, 131253, 131254, 131255, 131256, 131257, 131258, 131259 were selected from the ORFs having the nucleotide sequence represented by SEQ ID NO: 79672.

The probes having the nucleotide sequence represented by SEQ ID NOS: 131260, 131261, 131262, 131263, 131264, 131265, 131266, 131267, 131268, 131269, 131270 were selected from the ORFs having the nucleotide sequence represented by SEQ ID NO: 79673.

The probes having the nucleotide sequence represented by SEQ ID NOS: 131271, 131272, 131273, 131274, 131275, 131276, 131277, 131278, 131279, 131280, 131281 were selected from the ORFs having the nucleotide sequence represented by SEQ ID NO: 79674.

The probes having the nucleotide sequence represented by SEQ ID NOS: 131282, 131283, 131284, 131285, 131286, 131287, 131288, 131289, 131290, 131291, 131292 were selected from the ORFs having the nucleotide sequence represented by SEQ ID NO: 79675.

The probes having the nucleotide sequence represented by SEQ ID NOS: 131293, 131294, 131295, 131296, 131297, 131298, 131299, 131300, 131301, 131302, 131303 were selected from the ORFs having the nucleotide sequence represented by SEQ ID NO: 79676.

The probes having the nucleotide sequence represented by SEQ ID NOS: 131304, 131305, 131306, 131307, 131308, 131309, 131310, 131311, 131312, 131313, 131314 were selected from the ORFs having the nucleotide sequence represented by SEQ ID NO: 79677.

The probes having the nucleotide sequence represented by SEQ ID NOS: 131315, 131316, 131317, 131318, 131319, 131320, 131321, 131322, 131323, 131324, 131325 were selected from the ORFs having the nucleotide sequence represented by SEQ ID NO: 79678.

The probes having the nucleotide sequence represented by SEQ ID NOS: 131326, 131327, 131328, 131329, 131330, 131331, 131332, 131333, 131334, 131335, 131336 were selected from the ORFs having the nucleotide sequence represented by SEQ ID NO: 79679.

The probes having the nucleotide sequence represented by SEQ ID NOS: 131337, 131338, 131339, 131340, 131341, 131342, 131343, 131344, 131345, 131346, 131347 were selected from the ORFs having the nucleotide sequence represented by SEQ ID NO: 79680.

The probes having the nucleotide sequence represented by SEQ ID NOS: 131348, 131349, 131350, 131351, 131352, 131353, 131354, 131355, 131356, 131357, 131358 were selected from the ORFs having the nucleotide sequence represented by SEQ ID NO: 79681.

The probes having the nucleotide sequence represented by SEQ ID NOS: 131359, 131360, 131361, 131362, 131363, 131364, 131365, 131366, 131367, 131368, 131369 were selected from the ORFs having the nucleotide sequence represented by SEQ ID NO: 79682.

The probes having the nucleotide sequence represented by SEQ ID NOS: 131370, 131371, 131372, 131373, 131374, 131375, 131376, 131377, 131378, 131379, 131380 were selected from the ORFs having the nucleotide sequence represented by SEQ ID NO: 79683.

The probes having the nucleotide sequence represented by SEQ ID NOS: 131381, 131382, 131383, 131384, 131385, 131386, 131387, 131388, 131389, 131390, 131391 were selected from the ORFs having the nucleotide sequence represented by SEQ ID NO: 79684.

The probes having the nucleotide sequence represented by SEQ ID NOS: 131392, 131393, 131394, 131395, 131396, 131397, 131398, 131399, 131400, 131401, 131402 were selected from the ORFs having the nucleotide sequence represented by SEQ ID NO: 79685.

The probes having the nucleotide sequence represented by SEQ ID NOS: 131403, 131404, 131405, 131406, 131407, 131408, 131409, 131410, 131411, 131412, 131413 were selected from the ORFs having the nucleotide sequence represented by SEQ ID NO: 79686.

The probes having the nucleotide sequence represented by SEQ ID NOS: 131414, 131415, 131416, 131417, 131418, 131419, 131420, 131421, 131422, 131423, 131424 were selected from the ORFs having the nucleotide sequence represented by SEQ ID NO: 79687.

The probes having the nucleotide sequence represented by SEQ ID NOS: 131425, 131426, 131427, 131428, 131429, 131430, 131431, 131432, 131433, 131434, 131435 were selected from the ORFs having the nucleotide sequence represented by SEQ ID NO: 79688.

The probes having the nucleotide sequence represented by SEQ ID NOS: 131436, 131437, 131438, 131439, 131440, 131441, 131442, 131443, 131444, 131445, 131446 were selected from the ORFs having the nucleotide sequence represented by SEQ ID NO: 79689.

The probes having the nucleotide sequence represented by SEQ ID NOS: 131447, 131448, 131449, 131450, 131451, 131452, 131453, 131454, 131455, 131456, 131457 were selected from the ORFs having the nucleotide sequence represented by SEQ ID NO: 79690.

The probes having the nucleotide sequence represented by SEQ ID NOS: 131458, 131459, 131460, 131461, 131462, 131463, 131464, 131465, 131466, 131467, 131468 were selected from the ORFs having the nucleotide sequence represented by SEQ ID NO: 79691.

The probes having the nucleotide sequence represented by SEQ ID NOS: 131469, 131470, 131471, 131472, 131473, 131474, 131475, 131476, 131477, 131478, 131479 were selected from the ORFs having the nucleotide sequence represented by SEQ ID NO: 79692.

The probes having the nucleotide sequence represented by SEQ ID NOS: 131480, 131481, 131482, 131483, 131484, 131485, 131486, 131487, 131488, 131489, 131490 were selected from the ORFs having the nucleotide sequence represented by SEQ ID NO: 79693.

The probes having the nucleotide sequence represented by SEQ ID NOS: 131491, 131492, 131493, 131494, 131495, 131496, 131497, 131498, 131499, 131500, 131501 were selected from the ORFs having the nucleotide sequence represented by SEQ ID NO: 79694.

The probes having the nucleotide sequence represented by SEQ ID NOS: 131502, 131503, 131504, 131505, 131506, 131507, 131508, 131509, 131510, 131511, 131512 were selected from the ORFs having the nucleotide sequence represented by SEQ ID NO: 79695.

The probes having the nucleotide sequence represented by SEQ ID NOS: 131513, 131514, 131515, 131516, 131517, 131518, 131519, 131520, 131521, 131522, 131523 were selected from the ORFs having the nucleotide sequence represented by SEQ ID NO: 79696.

The probes having the nucleotide sequence represented by SEQ ID NOS: 131524, 131525, 131526, 131527, 131528, 131529, 131530, 131531, 131532, 131533, 131534 were selected from the ORFs having the nucleotide sequence represented by SEQ ID NO: 79697.

The probes having the nucleotide sequence represented by SEQ ID NOS: 131535, 131536, 131537, 131538, 131539, 131540, 131541, 131542, 131543, 131544, 131545 were selected from the ORFs having the nucleotide sequence represented by SEQ ID NO: 79698.

The probes having the nucleotide sequence represented by SEQ ID NOS: 131546, 131547, 131548, 131549, 131550, 131551, 131552, 131553, 131554, 131555, 131556 were selected from the ORFs having the nucleotide sequence represented by SEQ ID NO: 79699.

The probes having the nucleotide sequence represented by SEQ ID NOS: 131557, 131558, 131559, 131560, 131561, 131562, 131563, 131564, 131565, 131566, 131567 were selected from the ORFs having the nucleotide sequence represented by SEQ ID NO: 79700.

The probes having the nucleotide sequence represented by SEQ ID NOS: 131568, 131569, 131570, 131571, 131572, 131573, 131574, 131575, 131576, 131577, 131578 were selected from the ORFs having the nucleotide sequence represented by SEQ ID NO: 79701.

The probes having the nucleotide sequence represented by SEQ ID NOS: 131579, 131580, 131581, 131582, 131583, 131584, 131585, 131586, 131587, 131588, 131589 were selected from the ORFs having the nucleotide sequence represented by SEQ ID NO: 79702.

The probes having the nucleotide sequence represented by SEQ ID NOS: 131590, 131591, 131592, 131593, 131594, 131595, 131596, 131597, 131598, 131599, 131600 were selected from the ORFs having the nucleotide sequence represented by SEQ ID NO: 79703.

The probes having the nucleotide sequence represented by SEQ ID NOS: 131601, 131602, 131603, 131604, 131605, 131606, 131607, 131608, 131609, 131610, 131611 were selected from the ORFs having the nucleotide sequence represented by SEQ ID NO: 79704.

The probes having the nucleotide sequence represented by SEQ ID NOS: 131612, 131613, 131614, 131615, 131616, 131617, 131618, 131619, 131620, 131621, 131622 were selected from the ORFs having the nucleotide sequence represented by SEQ ID NO: 79705.

The probes having the nucleotide sequence represented by SEQ ID NOS: 131623, 131624, 131625, 131626, 131627, 131628, 131629, 131630, 131631, 131632, 131633 were selected from the ORFs having the nucleotide sequence represented by SEQ ID NO: 79706.

The probes having the nucleotide sequence represented by SEQ ID NOS: 131634, 131635, 131636, 131637, 131638, 131639, 131640, 131641, 131642, 131643, 131644 were selected from the ORFs having the nucleotide sequence represented by SEQ ID NO: 79707.

The probes having the nucleotide sequence represented by SEQ ID NOS: 131645, 131646, 131647, 131648, 131649, 131650, 131651, 131652, 131653, 131654, 131655 were selected from the ORFs having the nucleotide sequence represented by SEQ ID NO: 79708.

The probes having the nucleotide sequence represented by SEQ ID NOS: 131656, 131657, 131658, 131659, 131660, 131661, 131662, 131663, 131664, 131665, 131666 were selected from the ORFs having the nucleotide sequence represented by SEQ ID NO: 79709.

The probes having the nucleotide sequence represented by SEQ ID NOS: 131667, 131668, 131669, 131670, 131671, 131672, 131673, 131674, 131675, 131676, 131677 were selected from the ORFs having the nucleotide sequence represented by SEQ ID NO: 79710.

The probes having the nucleotide sequence represented by SEQ ID NOS: 131678, 131679, 131680, 131681, 131682, 131683, 131684, 131685, 131686, 131687, 131688 were selected from the ORFs having the nucleotide sequence represented by SEQ ID NO: 79711.

The probes having the nucleotide sequence represented by SEQ ID NOS: 131689, 131690, 131691, 131692, 131693, 131694, 131695, 131696, 131697, 131698, 131699 were selected from the ORFs having the nucleotide sequence represented by SEQ ID NO: 79712.

The probes having the nucleotide sequence represented by SEQ ID NOS: 131700, 131701, 131702, 131703, 131704, 131705, 131706, 131707, 131708, 131709, 131710 were selected from the ORFs having the nucleotide sequence represented by SEQ ID NO: 79713.

The probes having the nucleotide sequence represented by SEQ ID NOS: 131711, 131712, 131713, 131714, 131715, 131716, 131717, 131718, 131719, 131720, 131721 were selected from the ORFs having the nucleotide sequence represented by SEQ ID NO: 79714.

The probes having the nucleotide sequence represented by SEQ ID NOS: 131722, 131723, 131724, 131725, 131726, 131727, 131728, 131729, 131730, 131731, 131732 were selected from the ORFs having the nucleotide sequence represented by SEQ ID NO: 79715.

The probes having the nucleotide sequence represented by SEQ ID NOS: 131733, 131734, 131735, 131736, 131737, 131738, 131739, 131740, 131741, 131742, 131743 were selected from the ORFs having the nucleotide sequence represented by SEQ ID NO: 79716.

The probes having the nucleotide sequence represented by SEQ ID NOS: 131744, 131745, 131746, 131747, 131748, 131749, 131750, 131751, 131752, 131753, 131754 were selected from the ORFs having the nucleotide sequence represented by SEQ ID NO: 79717.

The probes having the nucleotide sequence represented by SEQ ID NOS: 131755, 131756, 131757, 131758, 131759, 131760, 131761, 131762, 131763, 131764, 131765 were selected from the ORFs having the nucleotide sequence represented by SEQ ID NO: 79718.

The probes having the nucleotide sequence represented by SEQ ID NOS: 131766, 131767, 131768, 131769, 131770, 131771, 131772, 131773, 131774, 131775, 131776 were selected from the ORFs having the nucleotide sequence represented by SEQ ID NO: 79719.

The probes having the nucleotide sequence represented by SEQ ID NOS: 131777, 131778, 131779, 131780, 131781, 131782, 131783, 131784, 131785, 131786, 131787 were selected from the ORFs having the nucleotide sequence represented by SEQ ID NO: 79720.

The probes having the nucleotide sequence represented by SEQ ID NOS: 131788, 131789, 131790, 131791, 131792, 131793, 131794, 131795, 131796, 131797, 131798 were selected from the ORFs having the nucleotide sequence represented by SEQ ID NO: 79721.

The probes having the nucleotide sequence represented by SEQ ID NOS: 131799, 131800, 131801, 131802, 131803, 131804, 131805, 131806, 131807, 131808, 131809 were selected from the ORFs having the nucleotide sequence represented by SEQ ID NO: 79722.

The probes having the nucleotide sequence represented by SEQ ID NOS: 131810, 131811, 131812, 131813, 131814, 131815, 131816, 131817, 131818, 131819, 131820 were selected from the ORFs having the nucleotide sequence represented by SEQ ID NO: 79723.

The probes having the nucleotide sequence represented by SEQ ID NOS: 131821, 131822, 131823, 131824, 131825, 131826, 131827, 131828, 131829, 131830, 131831 were selected from the ORFs having the nucleotide sequence represented by SEQ ID NO: 79724.

The probes having the nucleotide sequence represented by SEQ ID NOS: 131832, 131833, 131834, 131835, 131836, 131837, 131838, 131839, 131840, 131841, 131842 were selected from the ORFs having the nucleotide sequence represented by SEQ ID NO: 79725.

The probes having the nucleotide sequence represented by SEQ ID NOS: 131843, 131844, 131845, 131846, 131847, 131848, 131849, 131850, 131851, 131852, 131853 were selected from the ORFs having the nucleotide sequence represented by SEQ ID NO: 79726.

The probes having the nucleotide sequence represented by SEQ ID NOS: 131854, 131855, 131856, 131857, 131858, 131859, 131860, 131861, 131862, 131863, 131864 were selected from the ORFs having the nucleotide sequence represented by SEQ ID NO: 79727.

The probes having the nucleotide sequence represented by SEQ ID NOS: 131865, 131866, 131867, 131868, 131869, 131870, 131871, 131872, 131873, 131874, 131875 were selected from the ORFs having the nucleotide sequence represented by SEQ ID NO: 79728.

The probes having the nucleotide sequence represented by SEQ ID NOS: 131876, 131877, 131878, 131879, 131880, 131881, 131882, 131883, 131884, 131885, 131886 were selected from the ORFs having the nucleotide sequence represented by SEQ ID NO: 79729.

The probes having the nucleotide sequence represented by SEQ ID NOS: 131887, 131888, 131889, 131890, 131891, 131892, 131893, 131894, 131895, 131896, 131897 were selected from the ORFs having the nucleotide sequence represented by SEQ ID NO: 79730.

The probes having the nucleotide sequence represented by SEQ ID NOS: 131898, 131899, 131900, 131901, 131902, 131903, 131904, 131905, 131906, 131907, 131908 were selected from the ORFs having the nucleotide sequence represented by SEQ ID NO: 79731.

The probes having the nucleotide sequence represented by SEQ ID NOS: 131909, 131910, 131911, 131912, 131913, 131914, 131915, 131916, 131917, 131918, 131919 were selected from the ORFs having the nucleotide sequence represented by SEQ ID NO: 79732.

The probes having the nucleotide sequence represented by SEQ ID NOS: 131920, 131921, 131922, 131923, 131924, 131925, 131926, 131927, 131928, 131929, 131930 were selected from the ORFs having the nucleotide sequence represented by SEQ ID NO: 79733.

The probes having the nucleotide sequence represented by SEQ ID NOS: 131931, 131932, 131933, 131934, 131935, 131936, 131937, 131938, 131939, 131940, 131941 were selected from the ORFs having the nucleotide sequence represented by SEQ ID NO: 79734.

The probes having the nucleotide sequence represented by SEQ ID NOS: 131942, 131943, 131944, 131945, 131946, 131947, 131948, 131949, 131950, 131951, 131952 were selected from the ORFs having the nucleotide sequence represented by SEQ ID NO: 79735.

The probes having the nucleotide sequence represented by SEQ ID NOS: 131953, 131954, 131955, 131956, 131957, 131958, 131959, 131960, 131961, 131962, 131963 were selected from the ORFs having the nucleotide sequence represented by SEQ ID NO: 79736.

The probes having the nucleotide sequence represented by SEQ ID NOS: 131964, 131965, 131966, 131967, 131968, 131969, 131970, 131971, 131972, 131973, 131974 were selected from the ORFs having the nucleotide sequence represented by SEQ ID NO: 79737.

The probes having the nucleotide sequence represented by SEQ ID NOS: 131975, 131976, 131977, 131978, 131979, 131980, 131981, 131982, 131983, 131984, 131985 were selected from the ORFs having the nucleotide sequence represented by SEQ ID NO: 79738.

The probes having the nucleotide sequence represented by SEQ ID NOS: 131986, 131987, 131988, 131989, 131990, 131991, 131992, 131993, 131994, 131995, 131996 were selected from the ORFs having the nucleotide sequence represented by SEQ ID NO: 79739.

The probes having the nucleotide sequence represented by SEQ ID NOS: 131997, 131998, 131999, 132000, 132001, 132002, 132003, 132004, 132005, 132006, 132007 were selected from the ORFs having the nucleotide sequence represented by SEQ ID NO: 79740.

The probes having the nucleotide sequence represented by SEQ ID NOS: 132008, 132009, 132010, 132011, 132012, 132013, 132014, 132015, 132016, 132017, 132018 were selected from the ORFs having the nucleotide sequence represented by SEQ ID NO: 79741.

The probes having the nucleotide sequence represented by SEQ ID NOS: 132019, 132020, 132021, 132022, 132023, 132024, 132025, 132026, 132027, 132028, 132029 were selected from the ORFs having the nucleotide sequence represented by SEQ ID NO: 79742.

The probes having the nucleotide sequence represented by SEQ ID NOS: 132030, 132031, 132032, 132033, 132034, 132035, 132036, 132037, 132038, 132039, 132040 were selected from the ORFs having the nucleotide sequence represented by SEQ ID NO: 79743.

The probes having the nucleotide sequence represented by SEQ ID NOS: 132041, 132042, 132043, 132044, 132045, 132046, 132047, 132048, 132049, 132050, 132051 were selected from the ORFs having the nucleotide sequence represented by SEQ ID NO: 79744.

The probes having the nucleotide sequence represented by SEQ ID NOS: 132052, 132053, 132054, 132055, 132056, 132057, 132058, 132059, 132060, 132061, 132062 were selected from the ORFs having the nucleotide sequence represented by SEQ ID NO: 79745.

The probes having the nucleotide sequence represented by SEQ ID NOS: 132063, 132064, 132065, 132066, 132067, 132068, 132069, 132070, 132071, 132072, 132073 were selected from the ORFs having the nucleotide sequence represented by SEQ ID NO: 79746.

The probes having the nucleotide sequence represented by SEQ ID NOS: 132074, 132075, 132076, 132077, 132078, 132079, 132080, 132081, 132082, 132083, 132084 were selected from the ORFs having the nucleotide sequence represented by SEQ ID NO: 79747.

The probes having the nucleotide sequence represented by SEQ ID NOS: 132085, 132086, 132087, 132088, 132089, 132090, 132091, 132092, 132093, 132094, 132095 were selected from the ORFs having the nucleotide sequence represented by SEQ ID NO: 79748.

The probes having the nucleotide sequence represented by SEQ ID NOS: 132096, 132097, 132098, 132099, 132100, 132101, 132102, 132103, 132104, 132105, 132106 were selected from the ORFs having the nucleotide sequence represented by SEQ ID NO: 79749.

The probes having the nucleotide sequence represented by SEQ ID NOS: 132107, 132108, 132109, 132110, 132111, 132112, 132113, 132114, 132115, 132116, 132117 were selected from the ORFs having the nucleotide sequence represented by SEQ ID NO: 79750.

The probes having the nucleotide sequence represented by SEQ ID NOS: 132118, 132119, 132120, 132121, 132122, 132123, 132124, 132125, 132126, 132127, 132128 were selected from the ORFs having the nucleotide sequence represented by SEQ ID NO: 79751.

The probes having the nucleotide sequence represented by SEQ ID NOS: 132129, 132130, 132131, 132132, 132133, 132134, 132135, 132136, 132137, 132138, 132139 were selected from the ORFs having the nucleotide sequence represented by SEQ ID NO: 79752.

The probes having the nucleotide sequence represented by SEQ ID NOS: 132140, 132141, 132142, 132143, 132144, 132145, 132146, 132147, 132148, 132149, 132150 were selected from the ORFs having the nucleotide sequence represented by SEQ ID NO: 79753.

The probes having the nucleotide sequence represented by SEQ ID NOS: 132151, 132152, 132153, 132154, 132155, 132156, 132157, 132158, 132159, 132160, 132161 were selected from the ORFs having the nucleotide sequence represented by SEQ ID NO: 79754.

The probes having the nucleotide sequence represented by SEQ ID NOS: 132162, 132163, 132164, 132165, 132166, 132167, 132168, 132169, 132170, 132171, 132172 were selected from the ORFs having the nucleotide sequence represented by SEQ ID NO: 79755.

The probes having the nucleotide sequence represented by SEQ ID NOS: 132173, 132174, 132175, 132176, 132177, 132178, 132179, 132180, 132181, 132182, 132183 were selected from the ORFs having the nucleotide sequence represented by SEQ ID NO: 79756.

The probes having the nucleotide sequence represented by SEQ ID NOS: 132184, 132185, 132186, 132187, 132188, 132189, 132190, 132191, 132192, 132193, 132194 were selected from the ORFs having the nucleotide sequence represented by SEQ ID NO: 79757.

The probes having the nucleotide sequence represented by SEQ ID NOS: 132195, 132196, 132197, 132198, 132199, 132200, 132201, 132202, 132203, 132204, 132205 were selected from the ORFs having the nucleotide sequence represented by SEQ ID NO: 79758.

The probes having the nucleotide sequence represented by SEQ ID NOS: 132206, 132207, 132208, 132209, 132210, 132211, 132212, 132213, 132214, 132215, 132216 were selected from the ORFs having the nucleotide sequence represented by SEQ ID NO: 79759.

The probes having the nucleotide sequence represented by SEQ ID NOS: 132217, 132218, 132219, 132220, 132221, 132222, 132223, 132224, 132225, 132226, 132227 were selected from the ORFs having the nucleotide sequence represented by SEQ ID NO: 79760.

The probes having the nucleotide sequence represented by SEQ ID NOS: 132228, 132229, 132230, 132231, 132232, 132233, 132234, 132235, 132236, 132237, 132238 were selected from the ORFs having the nucleotide sequence represented by SEQ ID NO: 79761.

The probes having the nucleotide sequence represented by SEQ ID NOS: 132239, 132240, 132241, 132242, 132243, 132244, 132245, 132246, 132247, 132248, 132249, 132250, 132251, 132252, 132253, 132254, 132255, 132256, 132257, 132258, 132259, 132260 were selected from the ORFs having the nucleotide sequence represented by SEQ ID NO: 79762.

The probes having the nucleotide sequence represented by SEQ ID NOS: 132261, 132262, 132263, 132264, 132265, 132266, 132267, 132268, 132269, 132270, 132271 were selected from the ORFs having the nucleotide sequence represented by SEQ ID NO: 79763.

The probes having the nucleotide sequence represented by SEQ ID NOS: 132272, 132273, 132274, 132275, 132276, 132277, 132278, 132279, 132280, 132281, 132282 were selected from the ORFs having the nucleotide sequence represented by SEQ ID NO: 79764.

The probes having the nucleotide sequence represented by SEQ ID NOS: 132283, 132284, 132285, 132286, 132287, 132288, 132289, 132290, 132291, 132292, 132293 were selected from the ORFs having the nucleotide sequence represented by SEQ ID NO: 79765.

The probes having the nucleotide sequence represented by SEQ ID NOS: 132294, 132295, 132296, 132297, 132298, 132299, 132300, 132301, 132302, 132303, 132304 were selected from the ORFs having the nucleotide sequence represented by SEQ ID NO: 79766.

The probes having the nucleotide sequence represented by SEQ ID NOS: 132305, 132306, 132307, 132308, 132309, 132310, 132311, 132312, 132313, 132314, 132315 were selected from the ORFs having the nucleotide sequence represented by SEQ ID NO: 79767.

The probes having the nucleotide sequence represented by SEQ ID NOS: 132316, 132317, 132318, 132319, 132320, 132321, 132322, 132323, 132324, 132325, 132326 were selected from the ORFs having the nucleotide sequence represented by SEQ ID NO: 79768.

The probes having the nucleotide sequence represented by SEQ ID NOS: 132327, 132328, 132329, 132330, 132331, 132332, 132333, 132334, 132335, 132336, 132337 were selected from the ORFs having the nucleotide sequence represented by SEQ ID NO: 79769.

The probes having the nucleotide sequence represented by SEQ ID NOS: 132338, 132339, 132340, 132341, 132342, 132343, 132344, 132345, 132346, 132347, 132348 were selected from the ORFs having the nucleotide sequence represented by SEQ ID NO: 79770.

The probes having the nucleotide sequence represented by SEQ ID NOS: 132349, 132350, 132351, 132352, 132353, 132354, 132355, 132356, 132357, 132358, 132359 were selected from the ORFs having the nucleotide sequence represented by SEQ ID NO: 79771.

The probes having the nucleotide sequence represented by SEQ ID NOS: 132360, 132361, 132362, 132363, 132364, 132365, 132366, 132367, 132368, 132369, 132370 were selected from the ORFs having the nucleotide sequence represented by SEQ ID NO: 79772.

The probes having the nucleotide sequence represented by SEQ ID NOS: 132371, 132372, 132373, 132374, 132375, 132376, 132377, 132378, 132379, 132380, 132381 were selected from the ORFs having the nucleotide sequence represented by SEQ ID NO: 79773.

The probes having the nucleotide sequence represented by SEQ ID NOS: 132382, 132383, 132384, 132385, 132386, 132387, 132388, 132389, 132390, 132391, 132392 were selected from the ORFs having the nucleotide sequence represented by SEQ ID NO: 79774.

The probes having the nucleotide sequence represented by SEQ ID NOS: 132393, 132394, 132395, 132396, 132397, 132398, 132399, 132400, 132401, 132402, 132403 were selected from the ORFs having the nucleotide sequence represented by SEQ ID NO: 79775.

The probes having the nucleotide sequence represented by SEQ ID NOS: 132404, 132405, 132406, 132407, 132408, 132409, 132410, 132411, 132412, 132413, 132414 were selected from the ORFs having the nucleotide sequence represented by SEQ ID NO: 79776.

The probes having the nucleotide sequence represented by SEQ ID NOS: 132415, 132416, 132417, 132418, 132419, 132420, 132421, 132422, 132423, 132424, 132425 were selected from the ORFs having the nucleotide sequence represented by SEQ ID NO: 79777.

The probes having the nucleotide sequence represented by SEQ ID NOS: 132426, 132427, 132428, 132429, 132430, 132431, 132432, 132433, 132434, 132435, 132436 were selected from the ORFs having the nucleotide sequence represented by SEQ ID NO: 79778.

The probes having the nucleotide sequence represented by SEQ ID NOS: 132437, 132438, 132439, 132440, 132441, 132442, 132443, 132444, 132445, 132446, 132447 were selected from the ORFs having the nucleotide sequence represented by SEQ ID NO: 79779.

The probes having the nucleotide sequence represented by SEQ ID NOS: 132448, 132449, 132450, 132451, 132452, 132453, 132454, 132455, 132456, 132457, 132458 were selected from the ORFs having the nucleotide sequence represented by SEQ ID NO: 79780.

The probes having the nucleotide sequence represented by SEQ ID NOS: 132459, 132460, 132461, 132462, 132463, 132464, 132465, 132466, 132467, 132468, 132469 were selected from the ORFs having the nucleotide sequence represented by SEQ ID NO: 79781.

The probes having the nucleotide sequence represented by SEQ ID NOS: 132470, 132471, 132472, 132473, 132474, 132475, 132476, 132477, 132478, 132479, 132480 were selected from the ORFs having the nucleotide sequence represented by SEQ ID NO: 79782.

The probes having the nucleotide sequence represented by SEQ ID NOS: 132481, 132482, 132483, 132484, 132485, 132486, 132487, 132488, 132489, 132490, 132491 were selected from the ORFs having the nucleotide sequence represented by SEQ ID NO: 79783.

The probes having the nucleotide sequence represented by SEQ ID NOS: 132492, 132493, 132494, 132495, 132496, 132497, 132498, 132499, 132500, 132501, 132502 were selected from the ORFs having the nucleotide sequence represented by SEQ ID NO: 79784.

The probes having the nucleotide sequence represented by SEQ ID NOS: 132503, 132504, 132505, 132506, 132507, 132508, 132509, 132510, 132511, 132512, 132513 were selected from the ORFs having the nucleotide sequence represented by SEQ ID NO: 79785.

The probes having the nucleotide sequence represented by SEQ ID NOS: 132514, 132515, 132516, 132517, 132518, 132519, 132520, 132521, 132522, 132523, 132524 were selected from the ORFs having the nucleotide sequence represented by SEQ ID NO: 79786.

The probes having the nucleotide sequence represented by SEQ ID NOS: 132525, 132526, 132527, 132528, 132529, 132530, 132531, 132532, 132533, 132534, 132535 were selected from the ORFs having the nucleotide sequence represented by SEQ ID NO: 79787.

The probes having the nucleotide sequence represented by SEQ ID NOS: 132536, 132537, 132538, 132539, 132540, 132541, 132542, 132543, 132544, 132545, 132546 were selected from the ORFs having the nucleotide sequence represented by SEQ ID NO: 79788.

The probes having the nucleotide sequence represented by SEQ ID NOS: 132547, 132548, 132549, 132550, 132551, 132552, 132553, 132554, 132555, 132556, 132557, 132558, 132559, 132560, 132561, 132562, 132563, 132564, 132565, 132566, 132567, 132568 were selected from the ORFs having the nucleotide sequence represented by SEQ ID NO: 79789.

The probes having the nucleotide sequence represented by SEQ ID NOS: 132569, 132570, 132571, 132572, 132573, 132574, 132575, 132576, 132577, 132578, 132579 were selected from the ORFs having the nucleotide sequence represented by SEQ ID NO: 79790.

The probes having the nucleotide sequence represented by SEQ ID NOS: 132580, 132581, 132582, 132583, 132584, 132585, 132586, 132587, 132588, 132589, 132590 were selected from the ORFs having the nucleotide sequence represented by SEQ ID NO: 79791.

The probes having the nucleotide sequence represented by SEQ ID NOS: 132591, 132592, 132593, 132594, 132595, 132596, 132597, 132598, 132599, 132600, 132601 were selected from the ORFs having the nucleotide sequence represented by SEQ ID NO: 79792.

The probes having the nucleotide sequence represented by SEQ ID NOS: 132602, 132603, 132604, 132605, 132606, 132607, 132608, 132609, 132610, 132611, 132612 were selected from the ORFs having the nucleotide sequence represented by SEQ ID NO: 79793.

The probes having the nucleotide sequence represented by SEQ ID NOS: 132613, 132614, 132615, 132616, 132617, 132618, 132619, 132620, 132621, 132622, 132623 were selected from the ORFs having the nucleotide sequence represented by SEQ ID NO: 79794.

The probes having the nucleotide sequence represented by SEQ ID NOS: 132624, 132625, 132626, 132627, 132628, 132629, 132630, 132631, 132632, 132633, 132634, 132635, 132636, 132637, 132638, 132639, 132640, 132641, 132642, 132643, 132644, 132645 were selected from the ORFs having the nucleotide sequence represented by SEQ ID NO: 79795.

The probes having the nucleotide sequence represented by SEQ. ID NOS: 132646, 132647, 132648, 132649, 132650, 132651, 132652, 132653, 132654, 132655, 132656 were selected from the ORFs having the nucleotide sequence represented by SEQ ID NO: 79796.

The probes having the nucleotide sequence represented by SEQ ID NOS: 132657, 132658, 132659, 132660, 132661, 132662, 132663, 132664, 132665, 132666, 132667 were selected from the ORFs having the nucleotide sequence represented by SEQ ID NO: 79797.

The probes having the nucleotide sequence represented by SEQ ID NOS: 132668, 132669, 132670, 132671, 132672, 132673, 132674, 132675, 132676, 132677, 132678 were selected from the ORFs having the nucleotide sequence represented by SEQ ID NO: 79798.

The probes having the nucleotide sequence represented by SEQ ID NOS: 132679, 132680, 132681, 132682, 132683, 132684, 132685, 132686, 132687, 132688, 132689 were selected from the ORFs having the nucleotide sequence represented by SEQ ID NO: 79799.

The probes having the nucleotide sequence represented by SEQ ID NOS: 132690, 132691, 132692, 132693, 132694, 132695, 132696, 132697, 132698, 132699, 132700 were selected from the ORFs having the nucleotide sequence represented by SEQ ID NO: 79800.

The probes having the nucleotide sequence represented by SEQ ID NOS: 132701, 132702, 132703, 132704, 132705, 132706, 132707, 132708, 132709, 132710, 132711 were selected from the ORFs having the nucleotide sequence represented by SEQ ID NO: 79801.

The probes having the nucleotide sequence represented by SEQ ID NOS: 132712, 132713, 132714, 132715, 132716, 132717, 132718, 132719, 132720, 132721, 132722 were selected from the ORFs having the nucleotide sequence represented by SEQ ID NO: 79802.

The probes having the nucleotide sequence represented by SEQ ID NOS: 132723, 132724, 132725, 132726, 132727, 132728, 132729, 132730, 132731, 132732, 132733 were selected from the ORFs having the nucleotide sequence represented by SEQ ID NO: 79803.

The probes having the nucleotide sequence represented by SEQ ID NOS: 132734, 132735, 132736, 132737, 132738, 132739, 132740, 132741, 132742, 132743, 132744 were selected from the ORFs having the nucleotide sequence represented by SEQ ID NO: 79804.

The probes having the nucleotide sequence represented by SEQ ID NOS: 132745, 132746, 132747, 132748, 132749, 132750, 132751, 132752, 132753, 132754, 132755 were selected from the ORFs having the nucleotide sequence represented by SEQ ID NO: 79805.

The probes having the nucleotide sequence represented by SEQ ID NOS: 132756, 132757, 132758, 132759, 132760, 132761, 132762, 132763, 132764, 132765, 132766 were selected from the ORFs having the nucleotide sequence represented by SEQ ID NO: 79806.

The probes having the nucleotide sequence represented by SEQ ID NOS: 132767, 132768, 132769, 132770, 132771, 132772, 132773, 132774, 132775, 132776, 132777 were selected from the ORFs having the nucleotide sequence represented by SEQ ID NO: 79807.

The probes having the nucleotide sequence represented by SEQ ID NOS: 132778, 132779, 132780, 132781, 132782, 132783, 132784, 132785, 132786, 132787, 132788 were selected from the ORFs having the nucleotide sequence represented by SEQ ID NO: 79808.

The probes having the nucleotide sequence represented by SEQ ID NOS: 132789, 132790, 132791, 132792, 132793, 132794, 132795, 132796, 132797, 132798, 132799 were selected from the ORFs having the nucleotide sequence represented by SEQ ID NO: 79809.

The probes having the nucleotide sequence represented by SEQ ID NOS: 132800, 132801, 132802, 132803, 132804, 132805, 132806, 132807, 132808, 132809, 132810 were selected from the ORFs having the nucleotide sequence represented by SEQ ID NO: 79810.

The probes having the nucleotide sequence represented by SEQ ID NOS: 132811, 132812, 132813, 132814, 132815, 132816, 132817, 132818, 132819, 132820, 132821 were selected from the ORFs having the nucleotide sequence represented by SEQ ID NO: 79811.

The probes having the nucleotide sequence represented by SEQ ID NOS: 132822, 132823, 132824, 132825, 132826, 132827, 132828, 132829, 132830, 132831, 132832 were selected from the ORFs having the nucleotide sequence represented by SEQ ID NO: 79812.

The probes having the nucleotide sequence represented by SEQ ID NOS: 132833, 132834, 132835, 132836, 132837, 132838, 132839, 132840, 132841, 132842, 132843 were selected from the ORFs having the nucleotide sequence represented by SEQ ID NO: 79813.

The probes having the nucleotide sequence represented by SEQ ID NOS: 132844, 132845, 132846, 132847, 132848, 132849, 132850, 132851, 132852, 132853, 132854 were selected from the ORFs having the nucleotide sequence represented by SEQ ID NO: 79814.

The probes having the nucleotide sequence represented by SEQ ID NOS: 132855, 132856, 132857, 132858, 132859, 132860, 132861, 132862, 132863, 132864, 132865 were selected from the ORFs having the nucleotide sequence represented by SEQ ID NO: 79815.

The probes having the nucleotide sequence represented by SEQ ID NOS: 132866, 132867, 132868, 132869, 132870, 132871, 132872, 132873, 132874, 132875, 132876 were selected from the ORFs having the nucleotide sequence represented by SEQ ID NO: 79816.

The probes having the nucleotide sequence represented by SEQ ID NOS: 132877, 132878, 132879, 132880, 132881, 132882, 132883, 132884, 132885, 132886, 132887, 132888, 132889, 132890, 132891, 132892, 132893, 132894, 132895, 132896, 132897, 132898 were selected from the ORFs having the nucleotide sequence represented by SEQ ID NO: 79817.

The probes having the nucleotide sequence represented by SEQ ID NOS: 132899, 132900, 132901, 132902, 132903, 132904, 132905, 132906, 132907, 132908, 132909 were selected from the ORFs having the nucleotide sequence represented by SEQ ID NO: 79818.

The probes having the nucleotide sequence represented by SEQ ID NOS: 132910, 132911, 132912, 132913, 132914, 132915, 132916, 132917, 132918, 132919, 132920 were selected from the ORFs having the nucleotide sequence represented by SEQ ID NO: 79819.

The probes having the nucleotide sequence represented by SEQ ID NOS: 132921, 132922, 132923, 132924, 132925, 132926, 132927, 132928, 132929, 132930, 132931 were selected from the ORFs having the nucleotide sequence represented by SEQ ID NO: 79820.

The probes having the nucleotide sequence represented by SEQ ID NOS: 132932, 132933, 132934, 132935, 132936, 132937, 132938, 132939, 132940, 132941, 132942 were selected from the ORFs having the nucleotide sequence represented by SEQ ID NO: 79821.

The probes having the nucleotide sequence represented by SEQ ID NOS: 132943, 132944, 132945, 132946, 132947, 132948, 132949, 132950, 132951, 132952, 132953 were selected from the ORFs having the nucleotide sequence represented by SEQ ID NO: 79822.

The probes having the nucleotide sequence represented by SEQ ID NOS: 132954, 132955, 132956, 132957, 132958, 132959, 132960, 132961, 132962, 132963, 132964 were selected from the ORFs having the nucleotide sequence represented by SEQ ID NO: 79823.

The probes having the nucleotide sequence represented by SEQ ID NOS: 132965, 132966, 132967, 132968, 132969, 132970, 132971, 132972, 132973, 132974, 132975 were selected from the ORFs having the nucleotide sequence represented by SEQ ID NO: 79824.

The probes having the nucleotide sequence represented by SEQ ID NOS: 132976, 132977, 132978, 132979, 132980, 132981, 132982, 132983, 132984, 132985, 132986 were selected from the ORFs having the nucleotide sequence represented by SEQ ID NO: 79825.

The probes having the nucleotide sequence represented by SEQ ID NOS: 132987, 132988, 132989, 132990, 132991, 132992, 132993, 132994 were selected from the ORFs having the nucleotide sequence represented by SEQ ID NO: 79826.

The probes having the nucleotide sequence represented by SEQ ID NOS: 132995, 132996, 132997, 132998, 132999, 133000, 133001, 133002, 133003, 133004, 133005 were selected from the ORFs having the nucleotide sequence represented by SEQ ID NO: 79827.

The probes having the nucleotide sequence represented by SEQ ID NOS: 133006, 133007, 133008, 133009, 133010, 133011, 133012, 133013, 133014, 133015, 133016 were selected from the ORFs having the nucleotide sequence represented by SEQ ID NO: 79828.

The probes having the nucleotide sequence represented by SEQ ID NOS: 133017, 133018, 133019, 133020, 133021, 133022, 133023, 133024, 133025, 133026, 133027 were selected from the ORFs having the nucleotide sequence represented by SEQ ID NO: 79829.

The probes having the nucleotide sequence represented by SEQ ID NOS: 133028, 133029, 133030, 133031, 133032, 133033, 133034, 133035, 133036, 133037, 133038 were selected from the ORFs having the nucleotide sequence represented by SEQ ID NO: 79830.

The probes having the nucleotide sequence represented by SEQ ID NOS: 133039, 133040, 133041, 133042, 133043, 133044, 133045, 133046, 133047, 133048, 133049 were selected from the ORFs having the nucleotide sequence represented by SEQ ID NO: 79831.

The probes having the nucleotide sequence represented by SEQ ID NOS: 133050, 133051, 133052, 133053, 133054, 133055, 133056, 133057, 133058, 133059, 133060 were selected from the ORFs having the nucleotide sequence represented by SEQ ID NO: 79832.

The probes having the nucleotide sequence represented by SEQ ID NOS: 133061, 133062, 133063, 133064, 133065, 133066, 133067, 133068, 133069, 133070, 133071 were selected from the ORFs having the nucleotide sequence represented by SEQ ID NO: 79833.

The probes having the nucleotide sequence represented by SEQ ID NOS: 133072, 133073, 133074, 133075, 133076, 133077, 133078, 133079, 133080, 133081, 133082 were selected from the ORFs having the nucleotide sequence represented by SEQ ID NO: 79834.

The probes having the nucleotide sequence represented by SEQ ID NOS: 133083, 133084, 133085, 133086, 133087, 133088, 133089, 133090, 133091, 133092, 133093 were selected from the ORFs having the nucleotide sequence represented by SEQ ID NO: 79835.

The probes having the nucleotide sequence represented by SEQ ID NOS: 133094, 133095, 133096, 133097, 133098, 133099, 133100, 133101, 133102, 133103, 133104 were selected from the ORFs having the nucleotide sequence represented by SEQ ID NO: 79836.

The probes having the nucleotide sequence represented by SEQ ID NOS: 133105, 133106, 133107, 133108, 133109, 133110, 133111, 133112, 133113, 133114, 133115 were selected from the ORFs having the nucleotide sequence represented by SEQ ID NO: 79837.

The probes having the nucleotide sequence represented by SEQ ID NOS: 133116, 133117, 133118, 133119, 133120, 133121, 133122, 133123, 133124, 133125, 133126 were selected from the ORFs having the nucleotide sequence represented by SEQ ID NO: 79838.

The probes having the nucleotide sequence represented by SEQ ID NOS: 133127, 133128, 133129, 133130, 133131, 133132, 133133, 133134, 133135, 133136, 133137 were selected from the ORFs having the nucleotide sequence represented by SEQ ID NO: 79839.

The probes having the nucleotide sequence represented by SEQ ID NOS: 133138, 133139, 133140, 133141, 133142, 133143, 133144, 133145, 133146, 133147, 133148 were selected from the ORFs having the nucleotide sequence represented by SEQ ID NO: 79840.

The probes having the nucleotide sequence represented by SEQ ID NOS: 133149, 133150, 133151, 133152, 133153, 133154, 133155, 133156, 133157, 133158, 133159 were selected from the ORFs having the nucleotide sequence represented by SEQ ID NO: 79841.

The probes having the nucleotide sequence represented by SEQ ID NOS: 133160, 133161, 133162, 133163, 133164, 133165, 133166, 133167, 133168, 133169, 133170 were selected from the ORFs having the nucleotide sequence represented by SEQ ID NO: 79842.

The probes having the nucleotide sequence represented by SEQ ID NOS: 133171, 133172, 133173, 133174, 133175, 133176, 133177, 133178, 133179, 133180, 133181 were selected from the ORFs having the nucleotide sequence represented by SEQ ID NO: 79843.

The probes having the nucleotide sequence represented by SEQ ID NOS: 133182, 133183, 133184, 133185, 133186, 133187, 133188, 133189, 133190, 133191, 133192 were selected from the ORFs having the nucleotide sequence represented by SEQ ID NO: 79844.

The probes having the nucleotide sequence represented by SEQ ID NOS: 133193, 133194, 133195, 133196, 133197, 133198, 133199, 133200, 133201, 133202, 133203 were selected from the ORFs having the nucleotide sequence represented by SEQ ID NO: 79845.

The probes having the nucleotide sequence represented by SEQ ID NOS: 133204,133205, 133206, 133207, 133208, 133209, 133210, 133211, 133212, 133213, 133214 were selected from the ORFs having the nucleotide sequence represented by SEQ ID NO: 79846.

The probes having the nucleotide sequence represented by SEQ ID NOS: 133215, 133216, 133217, 133218, 133219, 133220, 133221, 133222, 133223, 133224, 133225 were selected from the ORFs having the nucleotide sequence represented by SEQ ID NO: 79847.

The probes having the nucleotide sequence represented by SEQ ID NOS: 133226, 133227, 133228, 133229, 133230, 133231, 133232, 133233, 133234, 133235, 133236 were selected from the ORFs having the nucleotide sequence represented by SEQ ID NO: 79848.

The probes having the nucleotide sequence represented by SEQ ID NOS: 133237, 133238, 133239, 133240, 133241, 133242, 133243, 133244, 133245, 133246, 133247 were selected from the ORFs having the nucleotide sequence represented by SEQ ID NO: 79849.

The probes having the nucleotide sequence represented by SEQ ID NOS: 133248, 133249, 133250, 133251, 133252, 133253, 133254, 133255, 133256, 133257, 133258 were selected from the ORFs having the nucleotide sequence represented by SEQ ID NO: 79850.

The probes having the nucleotide sequence represented by SEQ ID NOS: 133259, 133260, 133261, 133262, 133263, 133264, 133265, 133266, 133267, 133268, 133269 were selected from the ORFs having the nucleotide sequence represented by SEQ ID NO: 79851.

The probes having the nucleotide sequence represented by SEQ ID NOS: 133270, 133271, 133272, 133273, 133274, 133275, 133276, 133277, 133278, 133279, 133280 were selected from the ORFs having the nucleotide sequence represented by SEQ ID NO: 79852.

The probes having the nucleotide sequence represented by SEQ ID NOS: 133281, 133282, 133283, 133284, 133285, 133286, 133287, 133288, 133289, 133290, 133291 were selected from the ORFs having the nucleotide sequence represented by SEQ ID NO: 79853.

The probes having the nucleotide sequence represented by SEQ ID NOS: 133292, 133293, 133294, 133295, 133296, 133297, 133298, 133299, 133300, 133301, 133302 were selected from the ORFs having the nucleotide sequence represented by SEQ ID NO: 79854.

The probes having the nucleotide sequence represented by SEQ ID NOS: 133303, 133304, 133305, 133306, 133307, 133308, 133309, 133310, 133311, 133312, 133313 were selected from the ORFs having the nucleotide sequence represented by SEQ ID NO: 79855.

The probes having the nucleotide sequence represented by SEQ ID NOS: 133314, 133315, 133316, 133317, 133318, 133319, 133320, 133321, 133322, 133323, 133324 were selected from the ORFs having the nucleotide sequence represented by SEQ ID NO: 79856.

The probes having the nucleotide sequence represented by SEQ ID NOS: 133325, 133326, 133327, 133328, 133329, 133330, 133331, 133332, 133333, 133334, 133335 were selected from the ORFs having the nucleotide sequence represented by SEQ ID NO: 79857.

The probes having the nucleotide sequence represented by SEQ ID NOS: 133336, 133337, 133338, 133339, 133340, 133341, 133342, 133343, 133344, 133345, 133346 were selected from the ORFs having the nucleotide sequence represented by SEQ ID NO: 79858.

The probes having the nucleotide sequence represented by SEQ ID NOS: 133347, 133348, 133349, 133350, 133351, 133352, 133353, 133354, 133355, 133356, 133357 were selected from the ORFs having the nucleotide sequence represented by SEQ ID NO: 79859.

The probes having the nucleotide sequence represented by SEQ ID NOS: 133358, 133359, 133360, 133361, 133362, 133363, 133364, 133365, 133366, 133367, 133368 were selected from the ORFs having the nucleotide sequence represented by SEQ ID NO: 79860.

The probes having the nucleotide sequence represented by SEQ ID NOS: 133369, 133370, 133371, 133372, 133373, 133374, 133375, 133376, 133377, 133378, 133379 were selected from the ORFs having the nucleotide sequence represented by SEQ ID NO: 79861.

The probes having the nucleotide sequence represented by SEQ ID NOS: 133380, 133381, 133382, 133383, 133384, 133385, 133386, 133387, 133388, 133389, 133390 were selected from the ORFs having the nucleotide sequence represented by SEQ ID NO: 79862.

The probes having the nucleotide sequence represented by SEQ ID NOS: 133391, 133392, 133393, 133394, 133395, 133396, 133397, 133398, 133399, 133400, 133401 were selected from the ORFs having the nucleotide sequence represented by SEQ ID NO: 79863.

The probes having the nucleotide sequence represented by SEQ ID NOS: 133402, 133403, 133404, 133405, 133406, 133407, 133408, 133409, 133410, 133411, 133412 were selected from the ORFs having the nucleotide sequence represented by SEQ ID NO: 79864.

The probes having the nucleotide sequence represented by SEQ ID NOS: 133413, 133414, 133415, 133416, 133417, 133418, 133419, 133420, 133421, 133422, 133423 were selected from the ORFs having the nucleotide sequence represented by SEQ ID NO: 79865.

The probes having the nucleotide sequence represented by SEQ ID NOS: 133424, 133425, 133426, 133427, 133428, 133429, 133430, 133431, 133432, 133433, 133434 were selected from the ORFs having the nucleotide sequence represented by SEQ ID NO: 79866.

The probes having the nucleotide sequence represented by SEQ ID NOS: 133435, 133436, 133437, 133438, 133439, 133440, 133441, 133442, 133443, 133444, 133445 were selected from the ORFs having the nucleotide sequence represented by SEQ ID NO: 79867.

The probes having the nucleotide sequence represented by SEQ ID NOS: 133446, 133447, 133448, 133449, 133450, 133451, 133452, 133453, 133454, 133455, 133456 were selected from the ORFs having the nucleotide sequence represented by SEQ ID NO: 79868.

The probes having the nucleotide sequence represented by SEQ ID NOS: 133457, 133458, 133459, 133460, 133461, 133462, 133463, 133464, 133465, 133466, 133467 were selected from the ORFs having the nucleotide sequence represented by SEQ ID NO: 79869.

The probes having the nucleotide sequence represented by SEQ ID NOS: 133468, 133469, 133470, 133471, 133472, 133473, 133474, 133475, 133476, 133477, 133478 were selected from the ORFs having the nucleotide sequence represented by SEQ ID NO: 79870.

The probes having the nucleotide sequence represented by SEQ ID NOS: 133479, 133480, 133481, 133482, 133483, 133484, 133485, 133486, 133487, 133488, 133489 were selected from the ORFs having the nucleotide sequence represented by SEQ ID NO: 79871.

The probes having the nucleotide sequence represented by SEQ ID NOS: 133490, 133491, 133492, 133493, 133494, 133495, 133496, 133497, 133498, 133499, 133500 were selected from the ORFs having the nucleotide sequence represented by SEQ ID NO: 79872.

The probes having the nucleotide sequence represented by SEQ ID NOS: 133501, 133502, 133503, 133504, 133505, 133506, 133507, 133508, 133509, 133510, 133511 were selected from the ORFs having the nucleotide sequence represented by SEQ ID NO: 79873.

The probes having the nucleotide sequence represented by SEQ ID NOS: 133512, 133513, 133514, 133515, 133516, 133517, 133518, 133519, 133520, 133521, 133522 were selected from the ORFs having the nucleotide sequence represented by SEQ ID NO: 79874.

The probes having the nucleotide sequence represented by SEQ ID NOS: 133523, 133524, 133525, 133526, 133527, 133528, 133529, 133530, 133531, 133532, 133533 were selected from the ORFs having the nucleotide sequence represented by SEQ ID NO: 79875.

The probes having the nucleotide sequence represented by SEQ ID NOS: 133534, 133535, 133536, 133537, 133538, 133539, 133540, 133541, 133542, 133543, 133544 were selected from the ORFs having the nucleotide sequence represented by SEQ ID NO: 79876.

The probes having the nucleotide sequence represented by SEQ ID NOS: 133567, 133568, 133569, 133570, 133571, 133572, 133573, 133574, 133575, 133576, 133577 were selected from the ORFs having the nucleotide sequence represented by SEQ ID NO: 79877.

The probes having the nucleotide sequence represented by SEQ ID NOS: 133545, 133546, 133547, 133548, 133549, 133550, 133551, 133552, 133553, 133554, 133555 were selected from the ORFs having the nucleotide sequence represented by SEQ ID NO: 79878.

The probes having the nucleotide sequence represented by SEQ ID NOS: 133556, 133557, 133558, 133559, 133560, 133561, 133562, 133563, 133564, 133565, 133566 were selected from the ORFs having the nucleotide sequence represented by SEQ ID NO: 79879.

The probes having the nucleotide sequence represented by SEQ ID NOS: 133578, 133579, 133580, 133581, 133582, 133583, 133584, 133585, 133586, 133587, 133588 were selected from the ORFs having the nucleotide sequence represented by SEQ ID NO: 79880.

The probes having the nucleotide sequence represented by SEQ ID NOS: 133589, 133590, 133591, 133592, 133593, 133594, 133595, 133596, 133597, 133598, 133599 were selected from the ORFs having the nucleotide sequence represented by SEQ ID NO: 79881.

The probes having the nucleotide sequence represented by SEQ ID NOS: 133600, 133601, 133602, 133603, 133604, 133605, 133606, 133607, 133608, 133609, 133610 were selected from the ORFs having the nucleotide sequence represented by SEQ ID NO: 79882.

The probes having the nucleotide sequence represented by SEQ ID NOS: 133611, 133612, 133613, 133614, 133615, 133616, 133617, 133618, 133619, 133620, 133621 were selected from the ORFs having the nucleotide sequence represented by SEQ ID NO: 79883.

The probes having the nucleotide sequence represented by SEQ ID NOS: 133622, 133623, 133624, 133625, 133626, 133627, 133628, 133629, 133630, 133631, 133632 were selected from the ORFs having the nucleotide sequence represented by SEQ ID NO: 79884.

The probes having the nucleotide sequence represented by SEQ ID NOS: 133633, 133634, 133635, 133636, 133637, 133638, 133639, 133640, 133641, 133642, 133643 were selected from the ORFs having the nucleotide sequence represented by SEQ ID NO: 79885.

The probes having the nucleotide sequence represented by SEQ ID NOS: 133644, 133645, 133646, 133647, 133648, 133649, 133650, 133651, 133652, 133653, 133654 were selected from the ORFs having the nucleotide sequence represented by SEQ ID NO: 79886.

The probes having the nucleotide sequence represented by SEQ ID NOS: 133655, 133656, 133657, 133658, 133659, 133660, 133661, 133662, 133663, 133664, 133665 were selected from the ORFs having the nucleotide sequence represented by SEQ ID NO: 79887.

The probes having the nucleotide sequence represented by SEQ ID NOS: 133666, 133667, 133668, 133669, 133670, 133671, 133672, 133673, 133674, 133675, 133676 were selected from the ORFs having the nucleotide sequence represented by SEQ ID NO: 79888.

The probes having the nucleotide sequence represented by SEQ ID NOS: 133677, 133678, 133679, 133680, 133681, 133682, 133683, 133684, 133685, 133686, 133687 were selected from the ORFs having the nucleotide sequence represented by SEQ ID NO: 79889.

The probes having the nucleotide sequence represented by SEQ ID NOS: 133688, 133689, 133690, 133691, 133692, 133693, 133694, 133695, 133696, 133697, 133698 were selected from the ORFs having the nucleotide sequence represented by SEQ ID NO: 79890.

The probes having the nucleotide sequence represented by SEQ ID NOS: 133699, 133700, 133701, 133702, 133703, 133704, 133705, 133706, 133707, 133708, 133709 were selected from the ORFs having the nucleotide sequence represented by SEQ ID NO: 79891.

The probes having the nucleotide sequence represented by SEQ ID NOS: 133710, 133711, 133712, 133713, 133714, 133715, 133716, 133717, 133718, 133719, 133720 were selected from the ORFs having the nucleotide sequence represented by SEQ ID NO: 79892.

The probes having the nucleotide sequence represented by SEQ ID NOS: 133721, 133722, 133723, 133724, 133725, 133726, 133727, 133728, 133729, 133730, 133731 were selected from the ORFs having the nucleotide sequence represented by SEQ ID NO: 79893.

The probes having the nucleotide sequence represented by SEQ ID NOS: 133732, 133733, 133734, 133735, 133736, 133737, 133738, 133739, 133740, 133741, 133742 were selected from the ORFs having the nucleotide sequence represented by SEQ ID NO: 79894.

The probes having the nucleotide sequence represented by SEQ ID NOS: 133743, 133744, 133745, 133746, 133747, 133748, 133749, 133750, 133751, 133752, 133753 were selected from the ORFs having the nucleotide sequence represented by SEQ ID NO: 79895.

The probes having the nucleotide sequence represented by SEQ ID NOS: 133754, 133755, 133756, 133757, 133758, 133759, 133760, 133761, 133762, 133763, 133764 were selected from the ORFs having the nucleotide sequence represented by SEQ ID NO: 79896.

The probes having the nucleotide sequence represented by SEQ ID NOS: 133765, 133766, 133767, 133768, 133769, 133770, 133771, 133772, 133773, 133774, 133775 were selected from the ORFs having the nucleotide sequence represented by SEQ ID NO: 79897.

The probes having the nucleotide sequence represented by SEQ ID NOS: 133776, 133777, 133778, 133779, 133780, 133781, 133782, 133783, 133784, 133785, 133786 were selected from the ORFs having the nucleotide sequence represented by SEQ ID NO: 79898.

The probes having the nucleotide sequence represented by SEQ ID NOS: 133787, 133788, 133789, 133790, 133791, 133792, 133793, 133794, 133795, 133796, 133797 were selected from the ORFs having the nucleotide sequence represented by SEQ ID NO: 79899.

The probes having the nucleotide sequence represented by SEQ ID NOS: 133798, 133799, 133800, 133801, 133802, 133803, 133804, 133805, 133806, 133807, 133808 were selected from the ORFs having the nucleotide sequence represented by SEQ ID NO: 79900.

The probes having the nucleotide sequence represented by SEQ ID NOS: 133809, 133810, 133811, 133812, 133813, 133814, 133815, 133816, 133817, 133818, 133819 were selected from the ORFs having the nucleotide sequence represented by SEQ ID NO: 79901.

The probes having the nucleotide sequence represented by SEQ ID NOS: 133820, 133821, 133822, 133823, 133824, 133825, 133826, 133827, 133828, 133829, 133830 were selected from the ORFs having the nucleotide sequence represented by SEQ ID NO: 79902.

The probes having the nucleotide sequence represented by SEQ ID NOS: 133831, 133832, 133833, 133834, 133835, 133836, 133837, 133838, 133839, 133840, 133841 were selected from the ORFs having the nucleotide sequence represented by SEQ ID NO: 79903.

The probes having the nucleotide sequence represented by SEQ ID NOS: 133842, 133843, 133844, 133845, 133846, 133847, 133848, 133849, 133850, 133851, 133852 were selected from the ORFs having the nucleotide sequence represented by SEQ ID NO: 79904.

The probes having the nucleotide sequence represented by SEQ ID NOS: 133853, 133854, 133855, 133856, 133857, 133858, 133859, 133860, 133861, 133862, 133863 were selected from the ORFs having the nucleotide sequence represented by SEQ ID NO: 79905.

The probes having the nucleotide sequence represented by SEQ ID NOS: 133864, 133865, 133866, 133867, 133868, 133869, 133870, 133871, 133872, 133873, 133874 were selected from the ORFs having the nucleotide sequence represented by SEQ ID NO: 79906.

The probes having the nucleotide sequence represented by SEQ ID NOS: 133875, 133876, 133877, 133878, 133879, 133880, 133881, 133882, 133883, 133884, 133885 were selected from the ORFs having the nucleotide sequence represented by SEQ ID NO: 79907.

The probes having the nucleotide sequence represented by SEQ ID NOS: 133886, 133887, 133888, 133889, 133890, 133891, 133892, 133893, 133894, 133895, 133896 were selected from the ORFs having the nucleotide sequence represented by SEQ ID NO: 79908.

The probes having the nucleotide sequence represented by SEQ ID NOS: 133897, 133898, 133899, 133900, 133901, 133902, 133903, 133904, 133905, 133906, 133907 were selected from the ORFs having the nucleotide sequence represented by SEQ ID NO: 79909.

The probes having the nucleotide sequence represented by SEQ ID NOS: 133908, 133909, 133910, 133911, 133912, 133913, 133914, 133915, 133916, 133917, 133918 were selected from the ORFs having the nucleotide sequence represented by SEQ ID NO: 79910.

The probes having the nucleotide sequence represented by SEQ ID NOS: 133919, 133920, 133921, 133922, 133923, 133924, 133925, 133926, 133927, 133928, 133929 were selected from the ORFs having the nucleotide sequence represented by SEQ ID NO: 79911.

The probes having the nucleotide sequence represented by SEQ ID NOS: 133930, 133931, 133932, 133933, 133934, 133935, 133936, 133937, 133938, 133939, 133940 were selected from the ORFs having the nucleotide sequence represented by SEQ ID NO: 79912.

The probes having the nucleotide sequence represented by SEQ ID NOS: 133941, 13394.2, 133943, 133944, 133945, 133946, 133947, 133948, 133949, 133950, 133951 were selected from the ORFs having the nucleotide sequence represented by SEQ ID NO: 79913.

The probes having the nucleotide sequence represented by SEQ ID NOS: 133952, 133953, 133954, 133955, 133956, 133957, 133958, 133959, 133960, 133961, 133962 were selected from the ORFs having the nucleotide sequence represented by SEQ ID NO: 79914.

The probes having the nucleotide sequence represented by SEQ ID NOS: 133963, 133964, 133965, 133966, 133967, 133968, 133969, 133970, 133971, 133972, 133973 were selected from the ORFs having the nucleotide sequence represented by SEQ ID NO: 79915.

The probes having the nucleotide sequence represented by SEQ ID NOS: 133974, 133975, 133976, 133977, 133978, 133979, 133980, 133981, 133982, 133983, 133984 were selected from the ORFs having the nucleotide sequence represented by SEQ ID NO: 79916.

The probes having the nucleotide sequence represented by SEQ ID NOS: 133985, 133986, 133987, 133988, 133989, 133990, 133991, 133992, 133993, 133994, 133995 were selected from the ORFs having the nucleotide sequence represented by SEQ ID NO: 79917.

The probes having the nucleotide sequence represented by SEQ ID NOS: 133996, 133997, 133998, 133999, 134000, 134001, 134002, 134003, 134004, 134005, 134006 were selected from the ORFs having the nucleotide sequence represented by SEQ ID NO: 79918.

The probes having the nucleotide sequence represented by SEQ ID NOS: 134007, 134008, 134009, 134010, 134011, 134012, 134013, 134014, 134015, 134016, 134017 were selected from the ORFs having the nucleotide sequence represented by SEQ ID NO: 79919.

The probes having the nucleotide sequence represented by SEQ ID NOS: 134018, 134019, 134020, 134021, 134022, 134023, 134024, 134025, 134026, 134027, 134028 were selected from the ORFs having the nucleotide sequence represented by SEQ ID NO: 79920.

The probes having the nucleotide sequence represented by SEQ ID NOS: 134029, 134030, 134031, 134032, 134033, 134034, 134035, 134036, 134037, 134038, 134039 were selected from the ORFs having the nucleotide sequence represented by SEQ ID NO: 79921.

The probes having the nucleotide sequence represented by SEQ ID NOS: 134040, 134041, 134042, 134043, 134044, 134045, 134046, 134047, 134048, 134049, 134050 were selected from the ORFs having the nucleotide sequence represented by SEQ ID NO: 79922.

The probes having the nucleotide sequence represented by SEQ ID NOS: 134051, 134052, 134053, 134054, 134055, 134056, 134057, 134058, 134059, 134060, 134061 were selected from the ORFs having the nucleotide sequence represented by SEQ ID NO: 79923.

The probes having the nucleotide sequence represented by SEQ ID NOS: 134062, 134063, 134064, 134065, 134066, 134067, 134068, 134069, 134070, 134071, 134072 were selected from the ORFs having the nucleotide sequence represented by SEQ ID NO: 79924.

The probes having the nucleotide sequence represented by SEQ ID NOS: 134073, 134074, 134075, 134076, 134077, 134078, 134079, 134080, 134081, 134082, 134083 were selected from the ORFs having the nucleotide sequence represented by SEQ ID NO: 79925.

The probes having the nucleotide sequence represented by SEQ ID NOS: 134084, 134085, 134086, 134087, 134088, 134089, 134090, 134091, 134092, 134093, 134094 were selected from the ORFs having the nucleotide sequence represented by SEQ ID NO: 79926.

The probes having the nucleotide sequence represented by SEQ ID NOS: 134095, 134096, 134097, 134098, 134099, 134100, 134101, 134102, 134103, 134104, 134105 were selected from the ORFs having the nucleotide sequence represented by SEQ ID NO: 79927.

The probes having the nucleotide sequence represented by SEQ ID NOS: 134106, 134107, 134108, 134109, 134110, 134111, 134112, 134113, 134114, 134115, 134116 were selected from the ORFs having the nucleotide sequence represented by SEQ ID NO: 79928.

The probes having the nucleotide sequence represented by SEQ ID NOS: 134117, 134118, 134119, 134120, 134121, 134122, 134123, 134124, 134125, 134126, 134127 were selected from the ORFs having the nucleotide sequence represented by SEQ ID NO: 79929.

The probes having the nucleotide sequence represented by SEQ ID NOS: 134128, 134129, 134130, 134131, 134132, 134133, 134134, 134135, 134136, 134137, 134138 were selected from the ORFs having the nucleotide sequence represented by SEQ ID NO: 79930.

The probes having the nucleotide sequence represented by SEQ ID NOS: 134139, 134140, 134141, 134142, 134143, 134144, 134145, 134146, 134147, 134148, 134149 were selected from the ORFs having the nucleotide sequence represented by SEQ ID NO: 79931.

The probes having the nucleotide sequence represented by SEQ ID NOS: 134150, 134151, 134152, 134153, 134154, 134155, 134156, 134157, 134158, 134159, 134160 were selected from the ORFs having the nucleotide sequence represented by SEQ ID NO: 79932.

The probes having the nucleotide sequence represented by SEQ ID NOS: 134161, 134162, 134163, 134164, 134165, 134166, 134167, 134168, 134169, 134170, 134171 were selected from the ORFs having the nucleotide sequence represented by SEQ ID NO: 79933.

The probes having the nucleotide sequence represented by SEQ ID NOS: 134172, 134173, 134174, 134175, 134176, 134177, 134178, 134179, 134180, 134181, 134182 were selected from the ORFs having the nucleotide sequence represented by SEQ ID NO: 79934.

The probes having the nucleotide sequence represented by SEQ ID NOS: 134183, 134184, 134185, 134186, 134187, 134188, 134189, 134190, 134191, 134192, 134193 were selected from the ORFs having the nucleotide sequence represented by SEQ ID NO: 79935.

The probes having the nucleotide sequence represented by SEQ ID NOS: 134194, 134195, 134196, 134197, 134198, 134199, 134200, 134201, 134202, 134203, 134204 were selected from the ORFs having the nucleotide sequence represented by SEQ ID NO: 79936.

The probes having the nucleotide sequence represented by SEQ ID NOS: 134205, 134206, 134207, 134208, 134209, 134210, 134211, 134212, 134213, 134214, 134215 were selected from the ORFs having the nucleotide sequence represented by SEQ ID NO: 79937.

The probes having the nucleotide sequence represented by SEQ ID NOS: 134216, 134217, 134218, 134219, 134220, 134221, 134222, 134223, 134224, 134225, 134226 were selected from the ORFs having the nucleotide sequence represented by SEQ ID NO: 79938.

The probes having the nucleotide sequence represented by SEQ ID NOS: 134227, 134228, 134229, 134230, 134231, 134232, 134233, 134234, 134235, 134236, 134237 were selected from the ORFs having the nucleotide sequence represented by SEQ ID NO: 79939.

The probes having the nucleotide sequence represented by SEQ ID NOS: 134238, 134239, 134240, 134241, 134242, 134243, 134244, 134245, 134246, 134247, 134248 were selected from the ORFs having the nucleotide sequence represented by SEQ ID NO: 79940.

The probes having the nucleotide sequence represented by SEQ ID NOS: 134249, 134250, 134251, 134252, 134253, 134254, 134255, 134256, 134257, 134258, 134259 were selected from the ORFs having the nucleotide sequence represented by SEQ ID NO: 79941.

The probes having the nucleotide sequence represented by SEQ ID NOS: 134260, 134261, 134262, 134263, 134264, 134265, 134266, 134267, 134268, 134269, 134270 were selected from the ORFs having the nucleotide sequence represented by SEQ ID NO: 79942.

The probes having the nucleotide sequence represented by SEQ ID NOS: 134271, 134272, 134273, 134274, 134275, 134276, 134277, 134278, 134279, 134280, 134281 were selected from the ORFs having the nucleotide sequence represented by SEQ ID NO: 79943.

The probes having the nucleotide sequence represented by SEQ ID NOS: 134282, 134283, 134284, 134285, 134286, 134287, 134288, 134289, 134290, 134291, 134292 were selected from the ORFs having the nucleotide sequence represented by SEQ ID NO: 79944.

The probes having the nucleotide sequence represented by SEQ ID NOS: 134293, 134294, 134295, 134296, 134297, 134298, 134299, 134300, 134301, 134302, 134303 were selected from the ORFs having the nucleotide sequence represented by SEQ ID NO: 79945.

The probes having the nucleotide sequence represented by SEQ ID NOS: 134304, 134305, 134306, 134307, 134308, 134309, 134310, 134311, 134312, 134313, 134314 were selected from the ORFs having the nucleotide sequence represented by SEQ ID NO: 79946.

The probes having the nucleotide sequence represented by SEQ ID NOS: 134315, 134316, 134317, 134318, 134319, 134320, 134321, 134322, 134323, 134324, 134325 were selected from the ORFs having the nucleotide sequence represented by SEQ ID NO: 79947.

The probes having the nucleotide sequence represented by SEQ ID NOS: 134326, 134327, 134328, 134329, 134330, 134331, 134332, 134333, 134334, 134335, 134336 were selected from the ORFs having the nucleotide sequence represented by SEQ ID NO: 79948.

The probes having the nucleotide sequence represented by SEQ ID NOS: 134337, 134338, 134339, 134340, 134341, 134342, 134343, 134344, 134345, 134346, 134347 were selected from the ORFs having the nucleotide sequence represented by SEQ ID NO: 79949.

The probes having the nucleotide sequence represented by SEQ ID NOS: 134348, 134349, 134350, 134351, 134352, 134353, 134354, 134355, 134356, 134357, 134358 were selected from the ORFs having the nucleotide sequence represented by SEQ ID NO: 79950.

The probes having the nucleotide sequence represented by SEQ ID NOS: 134359, 134360, 134361, 134362, 134363, 134364, 134365, 134366, 134367, 134368, 134369 were selected from the ORFs having the nucleotide sequence represented by SEQ ID NO: 79951.

The probes having the nucleotide sequence represented by SEQ ID NOS: 134370, 134371, 134372, 134373, 134374, 134375, 134376, 134377, 134378, 134379, 134380 were selected from the ORFs having the nucleotide sequence represented by SEQ ID NO: 79952.

The probes having the nucleotide sequence represented by SEQ ID NOS: 134381, 134382, 134383, 134384, 134385, 134386, 134387, 134388, 134389, 134390, 134391 were selected from the ORFs having the nucleotide sequence represented by SEQ ID NO: 79953.

The probes having the nucleotide sequence represented by SEQ ID NOS: 134392, 134393, 134394, 134395, 134396, 134397, 134398, 134399, 134400, 134401, 134402 were selected from the ORFs having the nucleotide sequence represented by SEQ ID NO: 79954.

The probes having the nucleotide sequence represented by SEQ ID NOS: 134403, 134404, 134405, 134406, 134407, 134408, 134409, 134410, 134411, 134412, 134413 were selected from the ORFs having the nucleotide sequence represented by SEQ ID NO: 79955.

The probes having the nucleotide sequence represented by SEQ ID NOS: 134414, 134415, 134416, 134417, 134418, 134419, 134420, 134421, 134422, 134423, 134424 were selected from the ORFs having the nucleotide sequence represented by SEQ ID NO: 79956.

The probes having the nucleotide sequence represented by SEQ ID NOS: 134425, 134426, 134427, 134428, 134429, 134430, 134431, 134432, 134433, 134434, 134435 were selected from the ORFs having the nucleotide sequence represented by SEQ ID NO: 79957.

The probes having the nucleotide sequence represented by SEQ ID NOS: 134436, 134437, 134438, 134439, 134440, 134441, 134442, 134443, 134444, 134445, 134446 were selected from the ORFs having the nucleotide sequence represented by SEQ ID NO: 79958.

The probes having the nucleotide sequence represented by SEQ ID NOS: 134447, 134448, 134449, 134450, 134451, 134452, 134453, 134454, 134455, 134456, 134457 were selected from the ORFs having the nucleotide sequence represented by SEQ ID NO: 79959.

The probes having the nucleotide sequence represented by SEQ ID NOS: 134458, 134459, 134460, 134461, 134462, 134463, 134464, 134465, 134466, 134467, 134468 were selected from the ORFs having the nucleotide sequence represented by SEQ ID NO: 79960.

The probes having the nucleotide sequence represented by SEQ ID NOS: 134469, 134470, 134471, 134472, 134473, 134474, 134475, 134476, 134477, 134478, 134479 were selected from the ORFs having the nucleotide sequence represented by SEQ ID NO: 79961.

The probes having the nucleotide sequence represented by SEQ ID NOS: 134480, 134481, 134482, 134483, 134484, 134485, 134486, 134487, 134488, 134489, 134490 were selected from the ORFs having the nucleotide sequence represented by SEQ ID NO: 79962.

The probes having the nucleotide sequence represented by SEQ ID NOS: 134491, 134492, 134493, 134494, 134495, 134496, 134497, 134498, 134499, 134500, 134501 were selected from the ORFs having the nucleotide sequence represented by SEQ ID NO: 79963.

The probes having the nucleotide sequence represented by SEQ ID NOS: 134502, 134503, 134504, 134505, 134506, 134507, 134508, 134509, 134510, 134511, 134512 were selected from the ORFs having the nucleotide sequence represented by SEQ ID NO: 79964.

The probes having the nucleotide sequence represented by SEQ ID NOS: 134513, 134514, 134515, 134516, 134517, 134518, 134519, 134520, 134521, 134522, 134523 were selected from the ORFs having the nucleotide sequence represented by SEQ ID NO: 79965.

The probes having the nucleotide sequence represented by SEQ ID NOS: 134524, 134525, 134526, 134527, 134528, 134529, 134530, 134531, 134532, 134533, 134534 were selected from the ORFs having the nucleotide sequence represented by SEQ ID NO: 79966.

The probes having the nucleotide sequence represented by SEQ ID NOS: 134535, 134536, 134537, 134538, 134539, 134540, 134541, 134542, 134543, 134544, 134545 were selected from the ORFs having the nucleotide sequence represented by SEQ ID NO: 79967.

The probes having the nucleotide sequence represented by SEQ ID NOS: 134546, 134547, 134548, 134549, 134550, 134551, 134552, 134553, 134554, 134555, 134556 were selected from the ORFs having the nucleotide sequence represented by SEQ ID NO: 79968.

The probes having the nucleotide sequence represented by SEQ ID NOS: 134557, 134558, 134559, 134560, 134561, 134562, 134563, 134564, 134565, 134566, 134567 were selected from the ORFs having the nucleotide sequence represented by SEQ ID NO: 79969.

The probes having the nucleotide sequence represented by SEQ ID NOS: 134568, 134569, 134570, 134571, 134572, 134573, 134574, 134575, 134576, 134577, 134578 were selected from the ORFs having the nucleotide sequence represented by SEQ ID NO: 79970.

The probes having the nucleotide sequence represented by SEQ ID NOS: 134579, 134580, 134581, 134582, 134583, 134584, 134585, 134586, 134587, 134588, 134589 were selected from the ORFs having the nucleotide sequence represented by SEQ ID NO: 79971.

The probes having the nucleotide sequence represented by SEQ ID NOS: 134590, 134591, 134592, 134593, 134594, 134595, 134596, 134597, 134598, 134599, 134600 were selected from the ORFs having the nucleotide sequence represented by SEQ ID NO: 79972.

The probes having the nucleotide sequence represented by SEQ ID NOS: 134601, 134602, 134603, 134604, 134605, 134606, 134607, 134608, 134609, 134610, 134611 were selected from the ORFs having the nucleotide sequence represented by SEQ ID NO: 79973.

The probes having the nucleotide sequence represented by SEQ ID NOS: 134612, 134613, 134614, 134615, 134616, 134617, 134618, 134619, 134620, 134621, 134622 were selected from the ORFs having the nucleotide sequence represented by SEQ ID NO: 79974.

The probes having the nucleotide sequence represented by SEQ ID NOS: 134623, 134624, 134625, 134626, 134627, 134628, 134629, 134630, 134631, 134632, 134633 were selected from the ORFs having the nucleotide sequence represented by SEQ ID NO: 79975.

The probes having the nucleotide sequence represented by SEQ ID NOS: 134634, 134635, 134636, 134637, 134638, 134639, 134640, 134641, 134642, 134643, 134644 were selected from the ORFs having the nucleotide sequence represented by SEQ ID NO: 79976.

The probes having the nucleotide sequence represented by SEQ ID NOS: 134645, 134646, 134647, 134648, 134649, 134650, 134651, 134652, 134653, 134654, 134655 were selected from the ORFs having the nucleotide sequence represented by SEQ ID NO: 79977.

The probes having the nucleotide sequence represented by SEQ ID NOS: 134656, 134657, 134658, 134659, 134660, 134661, 134662, 134663, 134664, 134665, 134666 were selected from the ORFs having the nucleotide sequence represented by SEQ ID NO: 79978.

The probes having the nucleotide sequence represented by SEQ ID NOS: 134667, 134668, 134669, 134670, 134671, 134672, 134673, 134674, 134675, 134676, 134677 were selected from the ORFs having the nucleotide sequence represented by SEQ ID NO: 79979.

The probes having the nucleotide sequence represented by SEQ ID NOS: 134678, 134679, 134680, 134681, 134682, 134683, 134684, 134685, 134686, 134687, 134688 were selected from the ORFs having the nucleotide sequence represented by SEQ ID NO: 79980.

The probes having the nucleotide sequence represented by SEQ ID NOS: 134689, 134690, 134691, 134692, 134693, 134694, 134695, 134696, 134697, 134698, 134699 were selected from the ORFs having the nucleotide sequence represented by SEQ ID NO: 79981.

The probes having the nucleotide sequence represented by SEQ ID NOS: 134700, 134701, 134702, 134703, 134704, 134705, 134706, 134707, 134708, 134709, 134710 were selected from the ORFs having the nucleotide sequence represented by SEQ ID NO: 79982.

The probes having the nucleotide sequence represented by SEQ ID NOS: 134711, 134712, 134713, 134714, 134715, 134716, 134717, 134718, 134719, 134720, 134721 were selected from the ORFs having the nucleotide sequence represented by SEQ ID NO: 79983.

The probes having the nucleotide sequence represented by SEQ ID NOS: 134722, 134723, 134724, 134725, 134726, 134727, 134728, 134729, 134730, 134731, 134732 were selected from the ORFs having the nucleotide sequence represented by SEQ ID NO: 79984.

The probes having the nucleotide sequence represented by SEQ ID NOS: 134733, 134734, 134735, 134736, 134737, 134738, 134739, 134740, 134741, 134742, 134743 were selected from the ORFs having the nucleotide sequence represented by SEQ ID NO: 79985.

The probes having the nucleotide sequence represented by SEQ ID NOS: 134744, 134745, 134746, 134747, 134748, 134749, 134750, 134751, 134752, 134753, 134754 were selected from the ORFs having the nucleotide sequence represented by SEQ ID NO: 79986.

The probes having the nucleotide sequence represented by SEQ ID NOS: 134755, 134756, 134757, 134758, 134759, 134760, 134761, 134762, 134763, 134764, 134765 were selected from the ORFs having the nucleotide sequence represented by SEQ ID NO: 79987.

The probes having the nucleotide sequence represented by SEQ ID NOS: 134766, 134767, 134768, 134769, 134770, 134771, 134772, 134773, 134774, 134775, 134776 were selected from the ORFs having the nucleotide sequence represented by SEQ ID NO: 79988.

The probes having the nucleotide sequence represented by SEQ ID NOS: 134777, 134778, 134779, 134780, 134781, 134782, 134783, 134784, 134785, 134786, 134787 were selected from the ORFs having the nucleotide sequence represented by SEQ ID NO: 79989.

The probes having the nucleotide sequence represented by SEQ ID NOS: 134788, 134789, 134790, 134791, 134792, 134793, 134794, 134795, 134796, 134797, 134798 were selected from the ORFs having the nucleotide sequence represented by SEQ ID NO: 79990.

The probes having the nucleotide sequence represented by SEQ ID NOS: 134799, 134800, 134801, 134802, 134803, 134804, 134805, 134806, 134807, 134808, 134809 were selected from the ORFs having the nucleotide sequence represented by SEQ ID NO: 79991.

The probes having the nucleotide sequence represented by SEQ ID NOS: 134810, 134811, 134812, 134813, 134814, 134815, 134816, 134817, 134818, 134819, 134820 were selected from the ORFs having the nucleotide sequence represented by SEQ ID NO: 79992.

The probes having the nucleotide sequence represented by SEQ ID NOS: 134821, 134822, 134823, 134824, 134825, 134826, 134827, 134828, 134829, 134830, 134831 were selected from the ORFs having the nucleotide sequence represented by SEQ ID NO: 79993.

The probes having the nucleotide sequence represented by SEQ ID NOS: 134832, 134833, 134834, 134835, 134836, 134837, 134838, 134839, 134840, 134841, 134842 were selected from the ORFs having the nucleotide sequence represented by SEQ ID NO: 79994.

The probes having the nucleotide sequence represented by SEQ ID NOS: 134843, 134844, 134845, 134846, 134847, 134848, 134849, 134850, 134851, 134852, 134853 were selected from the ORFs having the nucleotide sequence represented by SEQ ID NO: 79995.

The probes having the nucleotide sequence represented by SEQ ID NOS: 134854, 134855, 134856, 134857, 134858, 134859, 134860, 134861, 134862, 134863, 134864 were selected from the ORFs having the nucleotide sequence represented by SEQ ID NO: 79996.

The probes having the nucleotide sequence represented by SEQ ID NOS: 134865, 134866, 134867, 134868, 134869, 134870, 134871, 134872, 134873, 134874, 134875 were selected from the ORFs having the nucleotide sequence represented by SEQ ID NO: 79997.

The probes having the nucleotide sequence represented by SEQ ID NOS: 134876, 134877, 134878, 134879, 134880, 134881, 134882, 134883, 134884, 134885, 134886 were selected from the ORFs having the nucleotide sequence represented by SEQ ID NO: 79998.

The probes having the nucleotide sequence represented by SEQ ID NOS: 134887, 134888, 134889, 134890, 134891, 134892, 134893, 134894, 134895, 134896, 134897 were selected from the ORFs having the nucleotide sequence represented by SEQ ID NO: 79999.

The probes having the nucleotide sequence represented by SEQ ID NOS: 134898, 134899, 134900, 134901, 134902, 134903, 134904, 134905, 134906, 134907, 134908 were selected from the ORFs having the nucleotide sequence represented by SEQ ID NO: 80000.

The probes having the nucleotide sequence represented by SEQ ID NOS: 134909, 134910, 134911, 134912, 134913, 134914, 134915, 134916, 134917, 134918, 134919 were selected from the ORFs having the nucleotide sequence represented by SEQ ID NO: 80001.

The probes having the nucleotide sequence represented by SEQ ID NOS: 134920, 134921, 134922, 134923, 134924, 134925, 134926, 134927, 134928, 134929, 134930 were selected from the ORFs having the nucleotide sequence represented by SEQ ID NO: 80002.

The probes having the nucleotide sequence represented by SEQ ID NOS: 134931, 134932, 134933, 134934, 134935, 134936, 134937, 134938, 134939, 134940, 134941 were selected from the ORFs having the nucleotide sequence represented by SEQ ID NO: 80003.

The probes having the nucleotide sequence represented by SEQ ID NOS: 134942, 134943, 134944, 134945, 134946, 134947, 134948, 134949, 134950, 134951, 134952 were selected from the ORFs having the nucleotide sequence represented by SEQ ID NO: 80004.

The probes having the nucleotide sequence represented by SEQ ID NOS: 134953, 134954, 134955, 134956, 134957, 134958, 134959, 134960, 134961, 134962, 134963 were selected from the ORFs having the nucleotide sequence represented by SEQ ID NO: 80005.

The probes having the nucleotide sequence represented by SEQ ID NOS: 134964, 134965, 134966, 134967, 134968, 134969, 134970, 134971, 134972, 134973, 134974 were selected from the ORFs having the nucleotide sequence represented by SEQ ID NO: 80006.

The probes having the nucleotide sequence represented by SEQ ID NOS: 134975, 134976, 134977, 134978, 134979, 134980, 134981, 134982, 134983, 134984, 134985 were selected from the ORFs having the nucleotide sequence represented by SEQ ID NO: 80007.

The probes having the nucleotide sequence represented by SEQ ID NOS: 134986, 134987, 134988, 134989, 134990, 134991, 134992, 134993, 134994, 134995, 134996 were selected from the ORFs having the nucleotide sequence represented by SEQ ID NO: 80008.

The probes having the nucleotide sequence represented by SEQ ID NOS: 134997, 134998, 134999, 135000, 135001, 135002, 135003, 135004, 135005, 135006, 135007 were selected from the ORFs having the nucleotide sequence represented by SEQ ID NO: 80009.

The probes having the nucleotide sequence represented by SEQ ID NOS: 135008, 135009, 135010, 135011, 135012, 135013, 135014, 135015, 135016, 135017, 135018 were selected from the ORFs having the nucleotide sequence represented by SEQ ID NO: 80010.

The probes having the nucleotide sequence represented by SEQ ID NOS: 135019, 135020, 135021, 135022, 135023, 135024, 135025, 135026, 135027, 135028, 135029 were selected from the ORFs having the nucleotide sequence represented by SEQ ID NO: 80011.

The probes having the nucleotide sequence represented by SEQ ID NOS: 135030, 135031, 135032, 135033, 135034, 135035, 135036, 135037, 135038, 135039, 135040 were selected from the ORFs having the nucleotide sequence represented by SEQ ID NO: 80012.

The probes having the nucleotide sequence represented by SEQ ID NOS: 135041, 135042, 135043, 135044, 135045, 135046, 135047, 135048, 135049, 135050, 135051 were selected from the ORFs having the nucleotide sequence represented by SEQ ID NO: 80013.

The probes having the nucleotide sequence represented by SEQ ID NOS: 135052, 135053, 135054, 135055, 135056, 135057, 135058, 135059, 135060, 135061, 135062 were selected from the ORFs having the nucleotide sequence represented by SEQ ID NO: 80014.

The probes having the nucleotide sequence represented by SEQ ID NOS: 135063, 135064, 135065, 135066, 135067, 135068, 135069, 135070, 135071, 135072, 135073 were selected from the ORFs having the nucleotide sequence represented by SEQ ID NO: 80015.

The probes having the nucleotide sequence represented by SEQ ID NOS: 135074, 135075, 135076, 135077, 135078, 135079, 135080, 135081, 135082, 135083, 135084 were selected from the ORFs having the nucleotide sequence represented by SEQ ID NO: 80016.

The probes having the nucleotide sequence represented by SEQ ID NOS: 135085, 135086, 135087, 135088, 135089, 135090, 135091, 135092, 135093, 135094, 135095 were selected from the ORFs having the nucleotide sequence represented by SEQ ID NO: 80017.

The probes having the nucleotide sequence represented by SEQ ID NOS: 135096, 135097, 135098, 135099, 135100, 135101, 135102, 135103, 135104, 135105, 135106 were selected from the ORFs having the nucleotide sequence represented by SEQ ID NO: 80018.

The probes having the nucleotide sequence represented by SEQ ID NOS: 135107, 135108, 135109, 135110, 135111, 135112, 135113, 135114, 135115, 135116, 135117 were selected from the ORFs having the nucleotide sequence represented by SEQ ID NO: 80019.

The probes having the nucleotide sequence represented by SEQ ID NOS: 135118, 135119, 135120, 135121, 135122, 135123, 135124, 135125, 135126, 135127, 135128 were selected from the ORFs having the nucleotide sequence represented by SEQ ID NO: 80020.

The probes having the nucleotide sequence represented by SEQ ID NOS: 135129, 135130, 135131, 135132, 135133, 135134, 135135, 135136, 135137, 135138, 135139 were selected from the ORFs having the nucleotide sequence represented by SEQ ID NO: 80021.

The probes having the nucleotide sequence represented by SEQ ID NOS: 135140, 135141, 135142, 135143, 135144, 135145, 135146, 135147, 135148, 135149, 135150 were selected from the ORFs having the nucleotide sequence represented by SEQ ID NO: 80022.

The probes having the nucleotide sequence represented by SEQ ID NOS: 135151, 135152, 135153, 135154, 135155, 135156, 135157, 135158, 135159, 135160, 135161 were selected from the ORFs having the nucleotide sequence represented by SEQ ID NO: 80023.

The probes having the nucleotide sequence represented by SEQ ID NOS: 135162, 135163, 135164, 135165, 135166, 135167, 135168, 135169, 135170, 135171, 135172 were selected from the ORFs having the nucleotide sequence represented by SEQ ID NO: 80024.

The probes having the nucleotide sequence represented by SEQ ID NOS: 135173, 135174, 135175, 135176, 135177, 135178, 135179, 135180, 135181, 135182, 135183 were selected from the ORFs having the nucleotide sequence represented by SEQ ID NO: 80025.

The probes having the nucleotide sequence represented by SEQ ID NOS: 135184, 135185, 135186, 135187, 135188, 135189, 135190, 135191, 135192, 135193, 135194 were selected from the ORFs having the nucleotide sequence represented by SEQ ID NO: 80026.

The probes having the nucleotide sequence represented by SEQ ID NOS: 135195, 135196, 135197, 135198, 135199, 135200, 135201, 135202, 135203, 135204, 135205 were selected from the ORFs having the nucleotide sequence represented by SEQ ID NO: 80027.

The probes having the nucleotide sequence represented by SEQ ID NOS: 135206, 135207, 135208, 135209, 135210, 135211, 135212, 135213, 135214, 135215, 135216 were selected from the ORFs having the nucleotide sequence represented by SEQ ID NO: 80028.

The probes having the nucleotide sequence represented by SEQ ID NOS: 135217, 135218, 135219, 135220, 135221, 135222, 135223, 135224, 135225, 135226, 135227 were selected from the ORFs having the nucleotide sequence represented by SEQ ID NO: 80029.

The probes having the nucleotide sequence represented by SEQ ID NOS: 135228, 135229, 135230, 135231, 135232, 135233, 135234, 135235, 135236, 135237, 135238 were selected from the ORFs having the nucleotide sequence represented by SEQ ID NO: 80030.

The probes having the nucleotide sequence represented by SEQ ID NOS: 135239, 135240, 135241, 135242, 135243, 135244, 135245, 135246, 135247, 135248, 135249 were selected from the ORFs having the nucleotide sequence represented by SEQ ID NO: 80031.

The probes having the nucleotide sequence represented by SEQ ID NOS: 135250, 135251, 135252, 135253, 135254, 135255, 135256, 135257, 135258, 135259, 135260 were selected from the ORFs having the nucleotide sequence represented by SEQ ID NO: 80032.

The probes having the nucleotide sequence represented by SEQ ID NOS: 135261, 135262, 135263, 135264, 135265, 135266, 135267, 135268, 135269, 135270, 135271 were selected from the ORFs having the nucleotide sequence represented by SEQ ID NO: 80033.

The probes having the nucleotide sequence represented by SEQ ID NOS: 135272, 135273, 135274, 135275, 135276, 135277, 135278, 135279, 135280, 135281, 135282 were selected from the ORFs having the nucleotide sequence represented by SEQ ID NO: 80034.

The probes having the nucleotide sequence represented by SEQ ID NOS: 135283, 135284, 135285, 135286, 135287, 135288, 135289, 135290, 135291, 135292, 135293 were selected from the ORFs having the nucleotide sequence represented by SEQ ID NO: 80035.

The probes having the nucleotide sequence represented by SEQ ID NOS: 135294, 135295, 135296, 135297, 135298, 135299, 135300, 135301, 135302, 135303, 135304 were selected from the ORFs having the nucleotide sequence represented by SEQ ID NO: 80036.

The probes having the nucleotide sequence represented by SEQ ID NOS: 135305, 135306, 135307, 135308, 135309, 135310, 135311, 135312, 135313, 135314, 135315 were selected from the ORFs having the nucleotide sequence represented by SEQ ID NO: 80037.

The probes having the nucleotide sequence represented by SEQ ID NOS: 135316, 135317, 135318, 135319, 135320, 135321, 135322, 135323, 135324, 135325, 135326 were selected from the ORFs having the nucleotide sequence represented by SEQ ID NO: 80038.

The probes having the nucleotide sequence represented by SEQ ID NOS: 135327, 135328, 135329, 135330, 135331, 135332, 135333, 135334, 135335, 135336, 135337 were selected from the ORFs having the nucleotide sequence represented by SEQ ID NO: 80039.

The probes having the nucleotide sequence represented by SEQ ID NOS: 135338, 135339, 135340, 135341, 135342, 135343, 135344, 135345, 135346, 135347, 135348 were selected from the ORFs having the nucleotide sequence represented by SEQ ID NO: 80040.

The probes having the nucleotide sequence represented by SEQ ID NOS: 135349, 135350, 135351, 135352, 135353, 135354, 135355, 135356, 135357, 135358, 135359 were selected from the ORFs having the nucleotide sequence represented by SEQ ID NO: 80041.

The probes having the nucleotide sequence represented by SEQ ID NOS: 135360, 135361, 135362, 135363, 135364, 135365, 135366, 135367, 135368, 135369, 135370 were selected from the ORFs having the nucleotide sequence represented by SEQ ID NO: 80042.

The probes having the nucleotide sequence represented by SEQ ID NOS: 135371, 135372, 135373, 135374, 135375, 135376, 135377, 135378, 135379, 135380, 135381 were selected from the ORFs having the nucleotide sequence represented by SEQ ID NO: 80043.

The probes having the nucleotide sequence represented by SEQ ID NOS: 135382, 135383, 135384, 135385, 135386, 135387, 135388, 135389, 135390, 135391, 135392 were selected from the ORFs having the nucleotide sequence represented by SEQ ID NO: 80044.

The probes having the nucleotide sequence represented by SEQ ID NOS: 135393, 135394, 135395, 135396, 135397, 135398, 135399, 135400, 135401, 135402, 135403 were selected from the ORFs having the nucleotide sequence represented by SEQ ID NO: 80045.

The probes having the nucleotide sequence represented by SEQ ID NOS: 135404, 135405, 135406, 135407, 135408, 135409, 135410, 135411, 135412, 135413, 135414 were selected from the ORFs having the nucleotide sequence represented by SEQ ID NO: 80046.

The probes having the nucleotide sequence represented by SEQ ID NOS: 135415, 135416, 135417, 135418, 135419, 135420, 135421, 135422, 135423, 135424, 135425 were selected from the ORFs having the nucleotide sequence represented by SEQ ID NO: 80047.

The probes having the nucleotide sequence represented by SEQ ID NOS: 135426, 135427, 135428, 135429, 135430, 135431, 135432, 135433, 135434, 135435, 135436 were selected from the ORFs having the nucleotide sequence represented by SEQ ID NO: 80048.

The probes having the nucleotide sequence represented by SEQ ID NOS: 135437, 135438, 135439, 135440, 135441, 135442, 135443, 135444, 135445, 135446, 135447 were selected from the ORFs having the nucleotide sequence represented by SEQ ID NO: 80049.

The probes having the nucleotide sequence represented by SEQ ID NOS: 135448, 135449, 135450, 135451, 135452, 135453, 135454, 135455, 135456, 135457, 135458 were selected from the ORFs having the nucleotide sequence represented by SEQ ID NO: 80050.

The probes having the nucleotide sequence represented by SEQ ID NOS: 135459, 135460, 135461, 135462, 135463, 135464, 135465, 135466, 135467, 135468, 135469 were selected from the ORFs having the nucleotide sequence represented by SEQ ID NO: 80051.

The probes having the nucleotide sequence represented by SEQ ID NOS: 135470, 135471, 135472, 135473, 135474, 135475, 135476, 135477, 135478, 135479, 135480 were selected from the ORFs having the nucleotide sequence represented by SEQ ID NO: 80052.

The probes having the nucleotide sequence represented by SEQ ID NOS: 135481, 135482, 135483, 135484, 135485, 135486, 135487, 135488, 135489, 135490, 135491 were selected from the ORFs having the nucleotide sequence represented by SEQ ID NO: 80053.

The probes having the nucleotide sequence represented by SEQ ID NOS: 135492, 135493, 135494, 135495, 135496, 135497, 135498, 135499, 135500, 135501, 135502 were selected from the ORFs having the nucleotide sequence represented by SEQ ID NO: 80054.

The probes having the nucleotide sequence represented by SEQ ID NOS: 135503, 135504, 135505, 135506, 135507, 135508, 135509, 135510, 135511, 135512, 135513 were selected from the ORFs having the nucleotide sequence represented by SEQ ID NO: 80055.

The probes having the nucleotide sequence represented by SEQ ID NOS: 135514, 135515, 135516, 135517, 135518, 135519, 135520, 135521, 135522, 135523, 135524 were selected from the ORFs having the nucleotide sequence represented by SEQ ID NO: 80056.

The probes having the nucleotide sequence represented by SEQ ID NOS: 135525, 135526, 135527, 135528, 135529, 135530, 135531, 135532, 135533, 135534, 135535 were selected from the ORFs having the nucleotide sequence represented by SEQ ID NO: 80057.

The probes having the nucleotide sequence represented by SEQ ID NOS: 135536, 135537, 135538, 135539, 135540, 135541, 135542, 135543, 135544, 135545, 135546 were selected from the ORFs having the nucleotide sequence represented by SEQ ID NO: 80058.

The probes having the nucleotide sequence represented by SEQ ID NOS: 135547, 135548, 135549, 135550, 135551, 135552, 135553, 135554, 135555, 135556, 135557 were selected from the ORFs having the nucleotide sequence represented by SEQ ID NO: 80059.

The probes having the nucleotide sequence represented by SEQ ID NOS: 135558, 135559, 135560, 135561, 135562, 135563, 135564, 135565, 135566, 135567, 135568 were selected from the ORFs having the nucleotide sequence represented by SEQ ID NO: 80060.

The probes having the nucleotide sequence represented by SEQ ID NOS: 135569, 135570, 135571, 135572, 135573, 135574, 135575, 135576, 135577, 135578, 135579 were selected from the ORFs having the nucleotide sequence represented by SEQ ID NO: 80061.

The probes having the nucleotide sequence represented by SEQ ID NOS: 135580, 135581, 135582, 135583, 135584, 135585, 135586, 135587, 135588, 135589, 135590 were selected from the ORFs having the nucleotide sequence represented by SEQ ID NO: 80062.

The probes having the nucleotide sequence represented by SEQ ID NOS: 135591, 135592, 135593, 135594, 135595, 135596, 135597, 135598, 135599, 135600, 135601 were selected from the ORFs having the nucleotide sequence represented by SEQ ID NO: 80063.

The probes having the nucleotide sequence represented by SEQ ID NOS: 135602, 135603, 135604, 135605, 135606, 135607, 135608, 135609, 135610, 135611, 135612 were selected from the ORFs having the nucleotide sequence represented by SEQ ID NO: 80064.

The probes having the nucleotide sequence represented by SEQ ID NOS: 135613, 135614, 135615, 135616, 135617, 135618, 135619, 135620, 135621, 135622, 135623 were selected from the ORFs having the nucleotide sequence represented by SEQ ID NO: 80065.

The probes having the nucleotide sequence represented by SEQ ID NOS: 135624, 135625, 135626, 135627, 135628, 135629, 135630, 135631, 135632, 135633, 135634 were selected from the ORFs having the nucleotide sequence represented by SEQ ID NO: 80066.

The probes having the nucleotide sequence represented by SEQ ID NOS: 135635, 135636, 135637, 135638, 135639, 135640, 135641, 135642, 135643, 135644, 135645 were selected from the ORFs having the nucleotide sequence represented by SEQ ID NO: 80067.

The probes having the nucleotide sequence represented by SEQ ID NOS: 135646, 135647, 135648, 135649, 135650, 135651, 135652, 135653, 135654, 135655, 135656 were selected from the ORFs having the nucleotide sequence represented by SEQ ID NO: 80068.

The probes having the nucleotide sequence represented by SEQ ID NOS: 135657, 135658, 135659, 135660, 135661, 135662, 135663, 135664, 135665, 135666, 135667 were selected from the ORFs having the nucleotide sequence represented by SEQ ID NO: 80069.

The probes having the nucleotide sequence represented by SEQ ID NOS: 135668, 135669, 135670, 135671, 135672, 135673, 135674, 135675, 135676, 135677, 135678 were selected from the ORFs having the nucleotide sequence represented by SEQ ID NO: 80070.

The probes having the nucleotide sequence represented by SEQ ID NOS: 135679, 135680, 135681, 135682, 135683, 135684, 135685, 135686, 135687, 135688, 135689 were selected from the ORFs having the nucleotide sequence represented by SEQ ID NO: 80071.

The probes having the nucleotide sequence represented by SEQ ID NOS: 135690, 135691, 135692, 135693, 135694, 135695, 135696, 135697, 135698, 135699, 135700 were selected from the ORFs having the nucleotide sequence represented by SEQ ID NO: 80072.

The probes having the nucleotide sequence represented by SEQ ID NOS: 135701, 135702, 135703, 135704, 135705, 135706, 135707, 135708, 135709, 135710, 135711 were selected from the ORFs having the nucleotide sequence represented by SEQ ID NO: 80073.

The probes having the nucleotide sequence represented by SEQ ID NOS: 135712, 135713, 135714, 135715, 135716, 135717, 135718, 135719, 135720, 135721, 135722 were selected from the ORFs having the nucleotide sequence represented by SEQ ID NO: 80074.

The probes having the nucleotide sequence represented by SEQ ID NOS: 135723, 135724, 135725, 135726, 135727, 135728, 135729, 135730, 135731, 135732, 135733 were selected from the ORFs having the nucleotide sequence represented by SEQ ID NO: 80075.

The probes having the nucleotide sequence represented by SEQ ID NOS: 135734, 135735, 135736, 135737, 135738, 135739, 135740, 135741, 135742, 135743, 135744 were selected from the ORFs having the nucleotide sequence represented by SEQ ID NO: 80076.

The probes having the nucleotide sequence represented by SEQ ID NOS: 135745, 135746, 135747, 135748, 135749, 135750, 135751, 135752, 135753, 135754, 135755 were selected from the ORFs having the nucleotide sequence represented by SEQ ID NO: 80077.

The probes having the nucleotide sequence represented by SEQ ID NOS: 135756, 135757, 135758, 135759, 135760, 135761, 135762, 135763, 135764, 135765, 135766 were selected from the ORFs having the nucleotide sequence represented by SEQ ID NO: 80078.

The probes having the nucleotide sequence represented by SEQ ID NOS: 135767, 135768, 135769, 135770, 135771, 135772, 135773, 135774, 135775, 135776, 135777 were selected from the ORFs having the nucleotide sequence represented by SEQ ID NO: 80079.

The probes having the nucleotide sequence represented by SEQ ID NOS: 135778, 135779, 135780, 135781, 135782, 135783, 135784, 135785, 135786, 135787, 135788 were selected from the ORFs having the nucleotide sequence represented by SEQ ID NO: 80080.

The probes having the nucleotide sequence represented by SEQ ID NOS: 135789, 135790, 135791, 135792, 135793, 135794, 135795, 135796, 135797, 135798, 135799 were selected from the ORFs having the nucleotide sequence represented by SEQ ID NO: 80081.

The probes having the nucleotide sequence represented by SEQ ID NOS: 135800, 135801, 135802, 135803, 135804, 135805, 135806, 135807, 135808, 135809, 135810 were selected from the ORFs having the nucleotide sequence represented by SEQ ID NO: 80082.

The probes having the nucleotide sequence represented by SEQ ID NOS: 135811, 135812, 135813, 135814, 135815, 135816, 135817, 135818, 135819, 135820, 135821 were selected from the ORFs having the nucleotide sequence represented by SEQ ID NO: 80083.

The probes having the nucleotide sequence represented by SEQ ID NOS: 135822, 135823, 135824, 135825, 135826, 135827, 135828, 135829, 135830, 135831, 135832 were selected from the ORFs having the nucleotide sequence represented by SEQ ID NO: 80084.

The probes having the nucleotide sequence represented by SEQ ID NOS: 135833, 135834, 135835, 135836, 135837, 135838, 135839, 135840, 135841, 135842, 135843 were selected from the ORFs having the nucleotide sequence represented by SEQ ID NO: 80085.

The probes having the nucleotide sequence represented by SEQ ID NOS: 135844, 135845, 135846, 135847, 135848, 135849, 135850, 135851, 135852, 135853, 135854, 135855, 135856, 135857, 135858, 135859, 135860, 135861, 135862, 135863, 135864, 135865 were selected from the ORFs having the nucleotide sequence represented by SEQ ID NO: 80086.

The probes having the nucleotide sequence represented by SEQ ID NOS: 135866, 135867, 135868, 135869, 135870, 135871, 135872, 135873, 135874, 135875, 135876 were selected from the ORFs having the nucleotide sequence represented by SEQ ID NO: 80087.

The probes having the nucleotide sequence represented by SEQ ID NOS: 135877, 135878, 135879, 135880, 135881, 135882, 135883, 135884, 135885, 135886, 135887 were selected from the ORFs having the nucleotide sequence represented by SEQ ID NO: 80088.

The probes having the nucleotide sequence represented by SEQ ID NOS: 135888, 135889, 135890, 135891, 135892, 135893, 135894, 135895, 135896, 135897, 135898 were selected from the ORFs having the nucleotide sequence represented by SEQ ID NO: 80089.

The probes having the nucleotide sequence represented by SEQ ID NOS: 135899, 135900, 135901, 135902, 135903, 135904, 135905, 135906, 135907, 135908, 135909 were selected from the ORFs having the nucleotide sequence represented by SEQ ID NO: 80090.

The probes having the nucleotide sequence represented by SEQ ID NOS: 135910, 135911, 135912, 135913, 135914, 135915, 135916, 135917, 135918, 135919, 135920 were selected from the ORFs having the nucleotide sequence represented by SEQ ID NO: 80091.

The probes having the nucleotide sequence represented by SEQ ID NOS: 135921, 135922, 135923, 135924, 135925, 135926, 135927, 135928, 135929, 135930, 135931 were selected from the ORFs having the nucleotide sequence represented by SEQ ID NO: 80092.

The probes having the nucleotide sequence represented by SEQ ID NOS: 135932, 135933, 135934, 135935, 135936, 135937, 135938, 135939, 135940, 135941, 135942 were selected from the ORFs having the nucleotide sequence represented by SEQ ID NO: 80093.

The probes having the nucleotide sequence represented by SEQ ID NOS: 135943, 135944, 135945, 135946, 135947, 135948, 135949, 135950, 135951, 135952, 135953 were selected from the ORFs having the nucleotide sequence represented by SEQ ID NO: 80094.

The probes having the nucleotide sequence represented by SEQ ID NOS: 135954, 135955, 135956, 135957, 135958, 135959, 135960, 135961, 135962, 135963, 135964 were selected from the ORFs having the nucleotide sequence represented by SEQ ID NO: 80095.

The probes having the nucleotide sequence represented by SEQ ID NOS: 135965, 135966, 135967, 135968, 135969, 135970, 135971, 135972, 135973, 135974, 135975 were selected from the ORFs having the nucleotide sequence represented by SEQ ID NO: 80096.

The probes having the nucleotide sequence represented by SEQ ID NOS: 135976, 135977, 135978, 135979, 135980, 135981, 135982, 135983, 135984, 135985, 135986 were selected from the ORFs having the nucleotide sequence represented by SEQ ID NO: 80097.

The probes having the nucleotide sequence represented by SEQ ID NOS: 135987, 135988, 135989, 135990, 135991, 135992, 135993, 135994, 135995, 135996, 135997 were selected from the ORFs having the nucleotide sequence represented by SEQ ID NO: 80098.

The probes having the nucleotide sequence represented by SEQ ID NOS: 135998, 135999, 136000, 136001, 136002, 136003, 136004, 136005, 136006, 136007, 136008 were selected from the ORFs having the nucleotide sequence represented by SEQ ID NO: 80099.

The probes having the nucleotide sequence represented by SEQ ID NOS: 136009, 136010, 136011, 136012, 136013, 136014, 136015, 136016, 136017, 136018, 136019 were selected from the ORFs having the nucleotide sequence represented by SEQ ID NO: 80100.

The probes having the nucleotide sequence represented by SEQ ID NOS: 136020, 136021, 136022, 136023, 136024, 136025, 136026, 136027, 136028, 136029, 136030 were selected from the ORFs having the nucleotide sequence represented by SEQ ID NO: 80101.

The probes having the nucleotide sequence represented by SEQ ID NOS: 136031, 136032, 136033, 136034, 136035, 136036, 136037, 136038, 136039, 136040, 136041 were selected from the ORFs having the nucleotide sequence represented by SEQ ID NO: 80102.

The probes having the nucleotide sequence represented by SEQ ID NOS: 136042, 136043, 136044, 136045, 136046, 136047, 136048, 136049, 136050, 136051, 136052 were selected from the ORFs having the nucleotide sequence represented by SEQ ID NO: 80103.

The probes having the nucleotide sequence represented by SEQ ID NOS: 136053, 136054, 136055, 136056, 136057, 136058, 136059, 136060, 136061, 136062, 136063 were selected from the ORFs having the nucleotide sequence represented by SEQ ID NO: 80104.

The probes having the nucleotide sequence represented by SEQ ID NOS: 136064, 136065, 136066, 136067, 136068, 136069, 136070, 136071, 136072, 136073, 136074 were selected from the ORFs having the nucleotide sequence represented by SEQ ID NO: 80105.

The probes having the nucleotide sequence represented by SEQ ID NOS: 136075, 136076, 136077, 136078, 136079, 136080, 136081, 136082, 136083, 136084, 136085 were selected from the ORFs having the nucleotide sequence represented by SEQ ID NO: 80106.

The probes having the nucleotide sequence represented by SEQ ID NOS: 136086, 136087, 136088, 136089, 136090, 136091, 136092, 136093, 136094, 136095, 136096 were selected from the ORFs having the nucleotide sequence represented by SEQ ID NO: 80107.

The probes having the nucleotide sequence represented by SEQ ID NOS: 136097, 136098, 136099, 136100, 136101, 136102, 136103, 136104, 136105, 136106, 136107 were selected from the ORFs having the nucleotide sequence represented by SEQ ID NO: 80108.

The probes having the nucleotide sequence represented by SEQ ID NOS: 136108, 136109, 136110, 136111, 136112, 136113, 136114, 136115, 136116, 136117, 136118, 136119, 136120, 136121, 136122, 136123, 136124, 136125, 136126, 136127, 136128, 136129 were selected from the ORFs having the nucleotide sequence represented by SEQ ID NO: 80109.

The probes having the nucleotide sequence represented by SEQ ID NOS: 136130, 136131, 136132, 136133, 136134, 136135, 136136, 136137, 136138, 136139, 136140 were selected from the ORFs having the nucleotide sequence represented by SEQ ID NO: 80110.

The probes having the nucleotide sequence represented by SEQ ID NOS: 136141, 136142, 136143, 136144, 136145, 136146, 136147, 136148, 136149, 136150, 136151 were selected from the ORFs having the nucleotide sequence represented by SEQ ID NO: 80111.

The probes having the nucleotide sequence represented by SEQ ID NOS: 136152, 136153, 136154, 136155, 136156, 136157, 136158, 136159, 136160, 136161, 136162 were selected from the ORFs having the nucleotide sequence represented by SEQ ID NO: 80112.

The probes having the nucleotide sequence represented by SEQ ID NOS: 136163, 136164, 136165, 136166, 136167, 136168, 136169, 136170, 136171, 136172, 136173 were selected from the ORFs having the nucleotide sequence represented by SEQ ID NO: 80113.

The probes having the nucleotide sequence represented by SEQ ID NOS: 136174, 136175, 136176, 136177, 136178, 136179, 136180, 136181, 136182, 136183, 136184 were selected from the ORFs having the nucleotide sequence represented by SEQ ID NO: 80114.

The probes having the nucleotide sequence represented by SEQ ID NOS: 136185, 136186, 136187, 136188, 136189, 136190, 136191, 136192, 136193, 136194, 136195 were selected from the ORFs having the nucleotide sequence represented by SEQ ID NO: 80115.

The probes having the nucleotide sequence represented by SEQ ID NOS: 136196, 136197, 136198, 136199, 136200, 136201, 136202, 136203, 136204, 136205, 136206 were selected from the ORFs having the nucleotide sequence represented by SEQ ID NO: 80116.

The probes having the nucleotide sequence represented by SEQ ID NOS: 136207, 136208, 136209, 136210, 136211, 136212, 136213, 136214, 136215, 136216, 136217 were selected from the ORFs having the nucleotide sequence represented by SEQ ID NO: 80117.

The probes having the nucleotide sequence represented by SEQ ID NOS: 136218, 136219, 136220, 136221, 136222, 136223, 136224, 136225, 136226, 136227, 136228 were selected from the ORFs having the nucleotide sequence represented by SEQ ID NO: 80118.

The probes having the nucleotide sequence represented by SEQ ID NOS: 136229, 136230, 136231, 136232, 136233, 136234, 136235, 136236, 136237, 136238, 136239 were selected from the ORFs having the nucleotide sequence represented by SEQ ID NO: 80119.

The probes having the nucleotide sequence represented by SEQ ID NOS: 136240, 136241, 136242, 136243, 136244, 136245, 136246, 136247, 136248, 136249, 136250 were selected from the ORFs having the nucleotide sequence represented by SEQ ID NO: 80120.

The probes having the nucleotide sequence represented by SEQ ID NOS: 136251, 136252, 136253, 136254, 136255, 136256, 136257, 136258, 136259, 136260, 136261 were selected from the ORFs having the nucleotide sequence represented by SEQ ID NO: 80121.

The probes having the nucleotide sequence represented by SEQ ID NOS: 136262, 136263, 136264, 136265, 136266, 136267, 136268, 136269, 136270, 136271, 136272 were selected from the ORFs having the nucleotide sequence represented by SEQ ID NO: 80122.

The probes having the nucleotide sequence represented by SEQ ID NOS: 136273, 136274, 136275, 136276, 136277, 136278, 136279, 136280, 136281, 136282, 136283 were selected from the ORFs having the nucleotide sequence represented by SEQ ID NO: 80123.

The probes having the nucleotide sequence represented by SEQ ID NOS: 136284, 136285, 136286, 136287, 136288, 136289, 136290, 136291, 136292, 136293, 136294 were selected from the ORFs having the nucleotide sequence represented by SEQ ID NO: 80124.

The probes having the nucleotide sequence represented by SEQ ID NOS: 136295, 136296, 136297, 136298, 136299, 136300, 136301, 136302, 136303, 136304, 136305 were selected from the ORFs having the nucleotide sequence represented by SEQ ID NO: 80125.

The probes having the nucleotide sequence represented by SEQ ID NOS: 136306, 136307, 136308, 136309, 136310, 136311, 136312, 136313, 136314, 136315, 136316 were selected from the ORFs having the nucleotide sequence represented by SEQ ID NO: 80126.

The probes having the nucleotide sequence represented by SEQ ID NOS: 136317, 136318, 136319, 136320, 136321, 136322, 136323, 136324, 136325, 136326, 136327 were selected from the ORFs having the nucleotide sequence represented by SEQ ID NO: 80127.

The probes having the nucleotide sequence represented by SEQ ID NOS: 136328, 136329, 136330, 136331, 136332, 136333, 136334, 136335, 136336, 136337; 136338, 136339, 136340, 136341, 136342, 136343, 136344, 136345, 136346, 136347, 136348, 136349 were selected from the ORFs having the nucleotide sequence represented by SEQ ID NO: 80128.

The probes having the nucleotide sequence represented by SEQ ID NOS: 136350, 136351, 136352, 136353, 136354, 136355, 136356, 136357, 136358, 136359, 136360, 136361, 136362, 136363, 136364, 136365, 136366, 136367, 136368, 136369, 136370, 136371 were selected from the ORFs having the nucleotide sequence represented by SEQ ID NO: 80129.

The probes having the nucleotide sequence represented by SEQ ID NOS: 136372, 136373, 136374, 136375, 136376, 136377, 136378, 136379, 136380, 136381, 136382 were selected from the ORFs having the nucleotide sequence represented by SEQ ID NO: 80130.

The probes having the nucleotide sequence represented by SEQ ID NOS: 136383, 136384, 136385, 136386, 136387, 136388, 136389, 136390, 136391, 136392, 136393 were selected from the ORFs having the nucleotide sequence represented by SEQ ID NO: 80131.

The probes having the nucleotide sequence represented by SEQ ID NOS: 136394, 136395, 136396, 136397, 136398, 136399, 136400, 136401, 136402, 136403, 136404 were selected from the ORFs having the nucleotide sequence represented by SEQ ID NO: 80132.

The probes having the nucleotide sequence represented by SEQ ID NOS: 136405, 136406, 136407, 136408, 136409, 136410, 136411, 136412, 136413, 136414, 136415 were selected from the ORFs having the nucleotide sequence represented by SEQ ID NO: 80133.

The probes having the nucleotide sequence represented by SEQ ID NOS: 136416, 136417, 136418, 136419, 136420, 136421, 136422, 136423, 136424, 136425, 136426 were selected from the ORFs having the nucleotide sequence represented by SEQ ID NO: 80134.

The probes having the nucleotide sequence represented by SEQ ID NOS: 136427, 136428, 136429, 136430, 136431, 136432, 136433, 136434, 136435, 136436, 136437 were selected from the ORFs having the nucleotide sequence represented by SEQ ID NO: 80135.

The probes having the nucleotide sequence represented by SEQ ID NOS: 136438, 136439, 136440, 136441, 136442, 136443, 136444, 136445, 136446, 136447, 136448 were selected from the ORFs having the nucleotide sequence represented by SEQ ID NO: 80136.

The probes having the nucleotide sequence represented by SEQ ID NOS: 136449, 136450, 136451, 136452, 136453, 136454, 136455, 136456, 136457, 136458, 136459 were selected from the ORFs having the nucleotide sequence represented by SEQ ID NO: 80137.

The probes having the nucleotide sequence represented by SEQ ID NOS: 136460, 136461, 136462, 136463, 136464, 136465, 136466, 136467, 136468, 136469, 136470, 136471, 136472, 136473, 136474, 136475, 136476, 136477, 136478, 136479, 136480, 136481 were selected from the ORFs having the nucleotide sequence represented by SEQ ID NO: 80138.

The probes having the nucleotide sequence represented by SEQ ID NOS: 136482, 136483, 136484, 136485, 136486, 136487, 136488, 136489, 136490, 136491, 136492 were selected from the ORFs having the nucleotide sequence represented by SEQ ID NO: 80139.

The probes having the nucleotide sequence represented by SEQ ID NOS: 136493, 136494, 136495, 136496, 136497, 136498, 136499, 136500, 136501, 136502, 136503 were selected from the ORFs having the nucleotide sequence represented by SEQ ID NO: 80140.

The probes having the nucleotide sequence represented by SEQ ID NOS: 136504, 136505, 136506, 136507, 136508, 136509, 136510, 136511, 136512, 136513, 136514 were selected from the ORFs having the nucleotide sequence represented by SEQ ID NO: 80141.

The probes having the nucleotide sequence represented by SEQ ID NOS: 136515, 136516, 136517, 136518, 136519, 136520, 136521, 136522, 136523, 136524, 136525 were selected from the ORFs having the nucleotide sequence represented by SEQ ID NO: 80142.

The probes having the nucleotide sequence represented by SEQ ID NOS: 136526, 136527, 136528, 136529, 136530, 136531, 136532, 136533, 136534, 136535 were selected from the ORFs having the nucleotide sequence represented by SEQ ID NO: 80143.

The probes having the nucleotide sequence represented by SEQ ID NOS: 136536, 136537, 136538, 136539, 136540, 136541, 136542, 136543, 136544, 136545, 136546 were selected from the ORFs having the nucleotide sequence represented by SEQ ID NO: 80144.

The probes having the nucleotide sequence represented by SEQ ID NOS: 136547, 136548, 136549, 136550, 136551, 136552, 136553, 136554, 136555, 136556, 136557 were selected from the ORFs having the nucleotide sequence represented by SEQ ID NO: 80145.

The probes having the nucleotide sequence represented by SEQ ID NOS: 136558, 136559, 136560, 136561, 136562, 136563, 136564, 136565, 136566, 136567, 136568 were selected from the ORFs having the nucleotide sequence represented by SEQ ID NO: 80146.

The probes having the nucleotide sequence represented by SEQ ID NOS: 136569, 136570, 136571, 136572, 136573, 136574, 136575, 136576, 136577, 136578, 136579 were selected from the ORFs having the nucleotide sequence represented by SEQ ID NO: 80147.

The probes having the nucleotide sequence represented by SEQ ID NOS: 136580, 136581, 136582, 136583, 136584, 136585, 136586, 136587, 136588, 136589, 136590 were selected from the ORFs having the nucleotide sequence represented by SEQ ID NO: 80148.

The probes having the nucleotide sequence represented by SEQ ID NOS: 136591, 136592, 136593, 136594, 136595, 136596, 136597, 136598, 136599, 136600, 136601 were selected from the ORFs having the nucleotide sequence represented by SEQ ID NO: 80149.

The probes having the nucleotide sequence represented by SEQ ID NOS: 136602, 136603, 136604, 136605, 136606, 136607, 136608, 136609, 136610, 136611, 136612 were selected from the ORFs having the nucleotide sequence represented by SEQ ID NO: 80150.

The probes having the nucleotide sequence represented by SEQ ID NOS: 136613, 136614, 136615, 136616, 136617, 136618, 136619, 136620, 136621, 136622, 136623 were selected from the ORFs having the nucleotide sequence represented by SEQ ID NO: 80151.

The probes having the nucleotide sequence represented by SEQ ID NOS: 136624, 136625, 136626, 136627, 136628, 136629, 136630, 136631, 136632, 136633, 136634 were selected from the ORFs having the nucleotide sequence represented by SEQ ID NO: 80152.

The probes having the nucleotide sequence represented by SEQ ID NOS: 136635, 136636, 136637, 136638, 136639, 136640, 136641, 136642, 136643, 136644, 136645 were selected from the ORFs having the nucleotide sequence represented by SEQ ID NO: 80153.

The probes having the nucleotide sequence represented by SEQ ID NOS: 136646, 136647, 136648, 136649, 136650, 136651, 136652, 136653, 136654, 136655, 136656 were selected from the ORFs having the nucleotide sequence represented by SEQ ID NO: 80154.

The probes having the nucleotide sequence represented by SEQ ID NOS: 136657, 136658, 136659, 136660, 136661, 136662, 136663, 136664, 136665, 136666, 136667 were selected from the ORFs having the nucleotide sequence represented by SEQ ID NO: 80155.

The probes having the nucleotide sequence represented by SEQ ID NOS: 136668, 136669, 136670, 136671, 136672, 136673, 136674, 136675, 136676, 136677, 136678 were selected from the ORFs having the nucleotide sequence represented by SEQ ID NO: 80156.

The probes having the nucleotide sequence represented by SEQ ID NOS: 136679, 136680, 136681, 136682, 136683, 136684, 136685, 136686, 136687, 136688, 136689 were selected from the ORFs having the nucleotide sequence represented by SEQ ID NO: 80157.

The probes having the nucleotide sequence represented by SEQ ID NOS: 136690, 136691, 136692, 136693, 136694, 136695, 136696, 136697, 136698, 136699, 136700 were selected from the ORFs having the nucleotide sequence represented by SEQ ID NO: 80158.

The probes having the nucleotide sequence represented by SEQ ID NOS: 136701, 136702, 136703, 136704, 136705, 136706, 136707, 136708, 136709, 136710, 136711 were selected from the ORFs having the nucleotide sequence represented by SEQ ID NO: 80159.

The probes having the nucleotide sequence represented by SEQ ID NOS: 136712, 136713, 136714, 136715, 136716, 136717, 136718, 136719, 136720, 136721, 136722 were selected from the ORFs having the nucleotide sequence represented by SEQ ID NO: 80160.

The probes having the nucleotide sequence represented by SEQ ID NOS: 136723, 136724, 136725, 136726, 136727, 136728, 136729, 136730, 136731, 136732, 136733 were selected from the ORFs having the nucleotide sequence represented by SEQ ID NO: 80161.

The probes having the nucleotide sequence represented by SEQ ID NOS: 136734, 136735, 136736, 136737, 136738, 136739, 136740, 136741, 136742, 136743, 136744 were selected from the ORFs having the nucleotide sequence represented by SEQ ID NO: 80162.

The probes having the nucleotide sequence represented by SEQ ID NOS: 136745, 136746, 136747, 136748, 136749, 136750, 136751, 136752, 136753, 136754, 136755 were selected from the ORFs having the nucleotide sequence represented by SEQ ID NO: 80163.

The probes having the nucleotide sequence represented by SEQ ID NOS: 136756, 136757, 136758, 136759, 136760, 136761, 136762, 136763, 136764, 136765, 136766 were selected from the ORFs having the nucleotide sequence represented by SEQ ID NO: 80164.

The probes having the nucleotide sequence represented by SEQ ID NOS: 136767, 136768, 136769, 136770, 136771, 136772, 136773, 136774, 136775, 136776, 136777 were selected from the ORFs having the nucleotide sequence represented by SEQ ID NO: 80165.

The probes having the nucleotide sequence represented by SEQ ID NOS: 136778, 136779, 136780, 136781, 136782, 136783, 136784, 136785, 136786, 136787, 136788 were selected from the ORFs having the nucleotide sequence represented by SEQ ID NO: 80166.

The probes having the nucleotide sequence represented by SEQ ID NOS: 136789, 136790, 136791, 136792, 136793, 136794, 136795, 136796, 136797, 136798, 136799 were selected from the ORFs having the nucleotide sequence represented by SEQ ID NO: 80167.

The probes having the nucleotide sequence represented by SEQ ID NOS: 136800, 136801, 136802, 136803, 136804, 136805, 136806, 136807, 136808, 136809, 136810 were selected from the ORFs having the nucleotide sequence represented by SEQ ID NO: 80168.

The probes having the nucleotide sequence represented by SEQ ID NOS: 136811, 136812, 136813, 136814, 136815, 136816, 136817, 136818, 136819, 136820, 136821 were selected from the ORFs having the nucleotide sequence represented by SEQ ID NO: 80169.

The probes having the nucleotide sequence represented by SEQ ID NOS: 136822, 136823, 136824, 136825, 136826, 136827, 136828, 136829, 136830, 136831, 136832 were selected from the ORFs having the nucleotide sequence represented by SEQ ID NO: 80170.

The probes having the nucleotide sequence represented by SEQ ID NOS: 136833, 136834, 136835, 136836, 136837, 136838, 136839, 136840, 136841, 136842, 136843 were selected from the ORFs having the nucleotide sequence represented by SEQ ID NO: 80171.

The probes having the nucleotide sequence represented by SEQ ID NOS: 136844, 136845, 136846, 136847, 136848, 136849, 136850, 136851, 136852, 136853, 136854 were selected from the ORFs having the nucleotide sequence represented by SEQ ID NO: 80172.

The probes having the nucleotide sequence represented by SEQ ID NOS: 136855, 136856, 136857, 136858, 136859, 136860, 136861, 136862, 136863, 136864, 136865 were selected from the ORFs having the nucleotide sequence represented by SEQ ID NO: 80173.

The probes having the nucleotide sequence represented by SEQ ID NOS: 136866, 136867, 136868, 136869, 136870, 136871, 136872, 136873, 136874, 136875, 136876 were selected from the ORFs having the nucleotide sequence represented by SEQ ID NO: 80174.

The probes having the nucleotide sequence represented by SEQ ID NOS: 136877, 136878, 136879, 136880, 136881, 136882, 136883, 136884, 136885, 136886, 136887 were selected from the ORFs having the nucleotide sequence represented by SEQ ID NO: 80175.

The probes having the nucleotide sequence represented by SEQ ID NOS: 136888, 136889, 136890, 136891, 136892, 136893, 136894, 136895, 136896, 136897, 136898 were selected from the ORFs having the nucleotide sequence represented by SEQ ID NO: 80176.

The probes having the nucleotide sequence represented by SEQ ID NOS: 136899, 136900, 136901, 136902, 136903, 136904, 136905, 136906, 136907, 136908, 136909 were selected from the ORFs having the nucleotide sequence represented by SEQ ID NO: 80177.

The probes having the nucleotide sequence represented by SEQ ID NOS: 136910, 136911, 136912, 136913, 136914, 136915, 136916, 136917, 136918, 136919, 136920 were selected from the ORFs having the nucleotide sequence represented by SEQ ID NO: 80178.

The probes having the nucleotide sequence represented by SEQ ID NOS: 136921, 136922, 136923, 136924, 136925, 136926, 136927, 136928, 136929, 136930, 136931 were selected from the ORFs having the nucleotide sequence represented by SEQ ID NO: 80179.

The probes having the nucleotide sequence represented by SEQ ID NOS: 136932, 136933, 136934, 136935, 136936, 136937, 136938, 136939, 136940, 136941, 136942 were selected from the ORFs having the nucleotide sequence represented by SEQ ID NO: 80180.

The probes having the nucleotide sequence represented by SEQ ID NOS: 136943, 136944, 136945, 136946, 136947, 136948, 136949, 136950, 136951, 136952, 136953 were selected from the ORFs having the nucleotide sequence represented by SEQ ID NO: 80181.

The probes having the nucleotide sequence represented by SEQ ID NOS: 136954, 136955, 136956, 136957, 136958, 136959, 136960, 136961, 136962, 136963, 136964 were selected from the ORFs having the nucleotide sequence represented by SEQ ID NO: 80182.

The probes having the nucleotide sequence represented by SEQ ID NOS: 136965, 136966, 136967, 136968, 136969, 136970, 136971, 136972, 136973, 136974, 136975 were selected from the ORFs having the nucleotide sequence represented by SEQ ID NO: 80183.

The probes having the nucleotide sequence represented by SEQ ID NOS: 136976, 136977, 136978, 136979, 136980, 136981, 136982, 136983, 136984, 136985, 136986 were selected from the ORFs having the nucleotide sequence represented by SEQ ID NO: 80184.

The probes having the nucleotide sequence represented by SEQ ID NOS: 136987, 136988, 136989, 136990, 136991, 136992, 136993, 136994, 136995, 136996, 136997 were selected from the ORFs having the nucleotide sequence represented by SEQ ID NO: 80185.

The probes having the nucleotide sequence represented by SEQ ID NOS: 136998, 136999, 137000, 137001, 137002, 137003, 137004, 137005, 137006, 137007, 137008 were selected from the ORFs having the nucleotide sequence represented by SEQ ID NO: 80186.

The probes having the nucleotide sequence represented by SEQ ID NOS: 137009, 137010, 137011, 137012, 137013, 137014, 137015, 137016, 137017, 137018, 137019 were selected from the ORFs having the nucleotide sequence represented by SEQ ID NO: 80187.

The probes having the nucleotide sequence represented by SEQ ID NOS: 137020, 137021, 137022, 137023, 137024, 137025, 137026, 137027, 137028, 137029, 137030 were selected from the ORFs having the nucleotide sequence represented by SEQ ID NO: 80188.

The probes having the nucleotide sequence represented by SEQ ID NOS: 137031, 137032, 137033, 137034, 137035, 137036, 137037, 137038, 137039, 137040, 137041 were selected from the ORFs having the nucleotide sequence represented by SEQ ID NO: 80189.

The probes having the nucleotide sequence represented by SEQ ID NOS: 137042, 137043, 137044, 137045, 137046, 137047, 137048, 137049, 137050, 137051, 137052 were selected from the ORFs having the nucleotide sequence represented by SEQ ID NO: 80190.

The probes having the nucleotide sequence represented by SEQ ID NOS: 137053, 137054, 137055, 137056, 137057, 137058, 137059, 137060, 137061, 137062, 137063 were selected from the ORFs having the nucleotide sequence represented by SEQ ID NO: 80191.

The probes having the nucleotide sequence represented by SEQ ID NOS: 137064, 137065, 137066, 137067, 137068, 137069, 137070, 137071, 137072, 137073, 137074 were selected from the ORFs having the nucleotide sequence represented by SEQ ID NO: 80192.

The probes having the nucleotide sequence represented by SEQ ID NOS: 137075, 137076, 137077, 137078, 137079, 137080, 137081, 137082, 137083, 137084, 137085, 137086, 137087, 137088, 137089, 137090, 137091, 137092, 137093, 137094, 137095, 137096 were selected from the ORFs having the nucleotide sequence represented by SEQ ID NO: 80193.

The probes having the nucleotide sequence represented by SEQ ID NOS: 137097, 137098, 137099, 137100, 137101, 137102, 137103, 137104, 137105, 137106, 137107 were selected from the ORFs having the nucleotide sequence represented by SEQ ID NO: 80194.

The probes having the nucleotide sequence represented by SEQ ID NOS: 137108, 137109, 137110, 137111, 137112, 137113, 137114, 137115, 137116, 137117, 137118 were selected from the ORFs having the nucleotide sequence represented by SEQ ID NO: 80195.

The probes having the nucleotide sequence represented by SEQ ID NOS: 137119, 137120, 137121, 137122, 137123, 137124, 137125, 137126, 137127, 137128, 137129 were selected from the ORFs having the nucleotide sequence represented by SEQ ID NO: 80196.

The probes having the nucleotide sequence represented by SEQ ID NOS: 137130, 137131, 137132, 137133, 137134, 137135, 137136, 137137, 137138, 137139, 137140 were selected from the ORFs having the nucleotide sequence represented by SEQ ID NO: 80197.

The probes having the nucleotide sequence represented by SEQ ID NOS: 137141, 137142, 137143, 137144, 137145, 137146, 137147, 137148, 137149, 137150, 137151 were selected from the ORFs having the nucleotide sequence represented by SEQ ID NO: 80198.

The probes having the nucleotide sequence represented by SEQ ID NOS: 137152, 137153, 137154, 137155, 137156, 137157, 137158, 137159, 137160, 137161, 137162 were selected from the ORFs having the nucleotide sequence represented by SEQ ID NO: 80199.

The probes having the nucleotide sequence represented by SEQ ID NOS: 137163, 137164, 137165, 137166, 137167, 137168, 137169, 137170, 137171, 137172, 137173 were selected from the ORFs having the nucleotide sequence represented by SEQ ID NO: 80200.

The probes having the nucleotide sequence represented by SEQ ID NOS: 137174, 137175, 137176, 137177, 137178, 137179, 137180, 137181, 137182, 137183, 137184 were selected from the ORFs having the nucleotide sequence represented by SEQ ID NO: 80201.

The probes having the nucleotide sequence represented by SEQ ID NOS: 137185, 137186, 137187, 137188, 137189, 137190, 137191, 137192, 137193, 137194, 137195 were selected from the ORFs having the nucleotide sequence represented by SEQ ID NO: 80202.

The probes having the nucleotide sequence represented by SEQ ID NOS: 137196, 137197, 137198, 137199, 137200, 137201, 137202, 137203, 137204, 137205, 137206 were selected from the ORFs having the nucleotide sequence represented by SEQ ID NO: 80203.

The probes having the nucleotide sequence represented by SEQ ID NOS: 137207, 137208, 137209, 137210, 137211, 137212, 137213, 137214, 137215, 137216, 137217 were selected from the ORFs having the nucleotide sequence represented by SEQ ID NO: 80204.

The probes having the nucleotide sequence represented by SEQ ID NOS: 137218, 137219, 137220, 137221, 137222, 137223, 137224, 137225, 137226, 137227, 137228 were selected from the ORFs having the nucleotide sequence represented by SEQ ID NO: 80205.

The probes having the nucleotide sequence represented by SEQ ID NOS: 137229, 137230, 137231, 137232, 137233, 137234, 137235, 137236, 137237, 137238, 137239 were selected from the ORFs having the nucleotide sequence represented by SEQ ID NO: 80206.

The probes having the nucleotide sequence represented by SEQ ID NOS: 137240, 137241, 137242, 137243, 137244, 137245, 137246, 137247, 137248, 137249, 137250 were selected from the ORFs having the nucleotide sequence represented by SEQ ID NO: 80207.

The probes having the nucleotide sequence represented by SEQ ID NOS: 137251, 137252, 137253, 137254, 137255, 137256, 137257, 137258, 137259, 137260, 137261 were selected from the ORFs having the nucleotide sequence represented by SEQ ID NO: 80208.

The probes having the nucleotide sequence represented by SEQ ID NOS: 137262, 137263, 137264, 137265, 137266, 137267, 137268, 137269, 137270, 137271, 137272 were selected from the ORFs having the nucleotide sequence represented by SEQ ID NO: 80209.

The probes having the nucleotide sequence represented by SEQ ID NOS: 137273, 137274, 137275, 137276, 137277, 137278, 137279, 137280, 137281, 137282, 137283 were selected from the ORFs having the nucleotide sequence represented by SEQ ID NO: 80210.

The probes having the nucleotide sequence represented by SEQ ID NOS: 137284, 137285, 137286, 137287, 137288, 137289, 137290, 137291, 137292, 137293, 137294 were selected from the ORFs having the nucleotide sequence represented by SEQ ID NO: 80211.

The probes having the nucleotide sequence represented by SEQ ID NOS: 137295, 137296, 137297, 137298, 137299, 137300, 137301, 137302, 137303, 137304, 137305 were selected from the ORFs having the nucleotide sequence represented by SEQ ID NO: 80212.

The probes having the nucleotide sequence represented by SEQ ID NOS: 137306, 137307, 137308, 137309, 137310, 137311, 137312, 137313, 137314, 137315, 137316 were selected from the ORFs having the nucleotide sequence represented by SEQ ID NO: 80213.

The probes having the nucleotide sequence represented by SEQ ID NOS: 137317, 137318, 137319, 137320, 137321, 137322, 137323, 137324, 137325, 137326, 137327, 137328, 137329, 137330, 137331, 137332, 137333, 137334, 137335, 137336, 137337, 137338 were selected from the ORFs having the nucleotide sequence represented by SEQ ID NO: 80214.

The probes having the nucleotide sequence represented by SEQ ID NOS: 137339, 137340, 137341, 137342, 137343, 137344, 137345, 137346, 137347, 137348, 137349 were selected from the ORFs having the nucleotide sequence represented by SEQ ID NO: 80215.

The probes having the nucleotide sequence represented by SEQ ID NOS: 137350, 137351, 137352, 137353, 137354, 137355, 137356, 137357, 137358, 137359, 137360 were selected from the ORFs having the nucleotide sequence represented by SEQ ID NO: 80216.

The probes having the nucleotide sequence represented by SEQ ID NOS: 137361, 137362, 137363, 137364, 137365, 137366, 137367, 137368, 137369, 137370, 137371 were selected from the ORFs having the nucleotide sequence represented by SEQ ID NO: 80217.

The probes having the nucleotide sequence represented by SEQ ID NOS: 137372, 137373, 137374, 137375, 137376, 137377, 137378, 137379, 137380, 137381, 137382 were selected from the ORFs having the nucleotide sequence represented by SEQ ID NO: 80218.

The probes having the nucleotide sequence represented by SEQ ID NOS: 137383, 137384, 137385, 137386, 137387, 137388, 137389, 137390, 137391, 137392, 137393 were selected from the ORFs having the nucleotide sequence represented by SEQ ID NO: 80219.

The probes having the nucleotide sequence represented by SEQ ID NOS: 137394, 137395, 137396, 137397, 137398, 137399, 137400, 137401, 137402, 137403, 137404 were selected from the ORFs having the nucleotide sequence represented by SEQ ID NO: 80220.

The probes having the nucleotide sequence represented by SEQ ID NOS: 137405, 137406, 137407, 137408, 137409, 137410, 137411, 137412, 137413, 137414, 137415 were selected from the ORFs having the nucleotide sequence represented by SEQ ID NO: 80221.

The probes having the nucleotide sequence represented by SEQ ID NOS: 137416, 137417, 137418, 137419, 137420, 137421, 137422, 137423, 137424, 137425, 137426 were selected from the ORFs having the nucleotide sequence represented by SEQ ID NO: 80222.

The probes having the nucleotide sequence represented by SEQ ID NOS: 137427, 137428, 137429, 137430, 137431, 137432, 137433, 137434, 137435, 137436, 137437 were selected from the ORFs having the nucleotide sequence represented by SEQ ID NO: 80223.

The probes having the nucleotide sequence represented by SEQ ID NOS: 137438, 137439, 137440, 137441, 137442, 137443, 137444, 137445, 137446, 137447, 137448, 137449, 137450, 137451, 137452, 137453, 137454, 137455, 137456, 137457, 137458, 137459 were selected from the ORFs having the nucleotide sequence represented by SEQ ID NO: 80224.

The probes having the nucleotide sequence represented by SEQ ID NOS: 137460, 137461, 137462, 137463, 137464, 137465, 137466, 137467, 137468, 137469, 137470 were selected from the ORFs having the nucleotide sequence represented by SEQ ID NO: 80225.

The probes having the nucleotide sequence represented by SEQ ID NOS: 137471, 137472, 137473, 137474, 137475, 137476, 137477, 137478, 137479, 137480, 137481 were selected from the ORFs having the nucleotide sequence represented by SEQ ID NO: 80226.

The probes having the nucleotide sequence represented by SEQ ID NOS: 137482, 137483, 137484, 137485, 137486, 137487, 137488, 137489, 137490, 137491, 137492 were selected from the ORFs having the nucleotide sequence represented by SEQ ID NO: 80227.

The probes having the nucleotide sequence represented by SEQ ID NOS: 137493, 137494, 137495, 137496, 137497, 137498, 137499, 137500, 137501, 137502, 137503, 137504, 137505, 137506, 137507, 137508, 137509, 137510, 137511, 137512, 137513, 137514 were selected from the ORFs having the nucleotide sequence represented by SEQ ID NO: 80228.

The probes having the nucleotide sequence represented by SEQ ID NOS: 137515, 137516, 137517, 137518, 137519, 137520, 137521, 137522, 137523, 137524, 137525 were selected from the ORFs having the nucleotide sequence represented by SEQ ID NO: 80229.

The probes having the nucleotide sequence represented by SEQ ID NOS: 137526, 137527, 137528, 137529, 137530, 137531, 137532, 137533, 137534, 137535, 137536 were selected from the ORFs having the nucleotide sequence represented by SEQ ID NO: 80230.

The probes having the nucleotide sequence represented by SEQ ID NOS: 137537, 137538, 137539, 137540, 137541, 137542, 137543, 137544, 137545, 137546, 137547 were selected from the ORFs having the nucleotide sequence represented by SEQ ID NO: 80231.

The probes having the nucleotide sequence represented by SEQ ID NOS: 137548, 137549, 137550, 137551, 137552, 137553, 137554, 137555, 137556, 137557, 137558 were selected from the ORFs having the nucleotide sequence represented by SEQ ID NO: 80232.

The probes having the nucleotide sequence represented by SEQ ID NOS: 137559, 137560, 137561, 137562, 137563, 137564, 137565, 137566, 137567, 137568, 137569 were selected from the ORFs having the nucleotide sequence represented by SEQ ID NO: 80233.

The probes having the nucleotide sequence represented by SEQ ID NOS: 137570, 137571, 137572, 137573, 137574, 137575, 137576, 137577, 137578, 137579, 137580 were selected from the ORFs having the nucleotide sequence represented by SEQ ID NO: 80234.

The probes having the nucleotide sequence represented by SEQ ID NOS: 137581, 137582, 137583, 137584, 137585, 137586, 137587, 137588, 137589, 137590, 137591 were selected from the ORFs having the nucleotide sequence represented by SEQ ID NO: 80235.

The probes having the nucleotide sequence represented by SEQ ID NOS: 137592, 137593, 137594, 137595, 137596, 137597, 137598, 137599, 137600, 137601, 137602 were selected from the ORFs having the nucleotide sequence represented by SEQ ID NO: 80236.

The probes having the nucleotide sequence represented by SEQ ID NOS: 137603, 137604, 137605, 137606, 137607, 137608, 137609, 137610, 137611, 137612, 137613 were selected from the ORFs having the nucleotide sequence represented by SEQ ID NO: 80237.

The probes having the nucleotide sequence represented by SEQ ID NOS: 137614, 137615, 137616, 137617, 137618, 137619, 137620, 137621, 137622, 137623, 137624 were selected from the ORFs having the nucleotide sequence represented by SEQ ID NO: 80238.

The probes having the nucleotide sequence represented by SEQ ID NOS: 137625, 137626, 137627, 137628, 137629, 137630, 137631, 137632, 137633, 137634, 137635 were selected from the ORFs having the nucleotide sequence represented by SEQ ID NO: 80239.

The probes having the nucleotide sequence represented by SEQ ID NOS: 137636, 137637, 137638, 137639, 137640, 137641, 137642, 137643, 137644, 137645, 137646 were selected from the ORFs having the nucleotide sequence represented by SEQ ID NO: 80240.

The probes having the nucleotide sequence represented by SEQ ID NOS: 137647, 137648, 137649, 137650, 137651, 137652, 137653, 137654, 137655, 137656, 137657 were selected from the ORFs having the nucleotide sequence represented by SEQ ID NO: 80241.

The probes having the nucleotide sequence represented by SEQ ID NOS: 137658, 137659, 137660, 137661, 137662, 137663, 137664, 137665, 137666, 137667, 137668 were selected from the ORFs having the nucleotide sequence represented by SEQ ID NO: 80242.

The probes having the nucleotide sequence represented by SEQ ID NOS: 137669, 137670, 137671, 137672, 137673, 137674, 137675, 137676, 137677, 137678, 137679 were selected from the ORFs having the nucleotide sequence represented by SEQ ID NO: 80243.

The probes having the nucleotide sequence represented by SEQ ID NOS: 137680, 137681, 137682, 137683, 137684, 137685, 137686, 137687, 137688, 137689, 137690 were selected from the ORFs having the nucleotide sequence represented by SEQ ID NO: 80244.

The probes having the nucleotide sequence represented by SEQ ID NOS: 137691, 137692, 137693, 137694, 137695, 137696, 137697, 137698, 137699, 137700, 137701 were selected from the ORFs having the nucleotide sequence represented by SEQ ID NO: 80245.

The probes having the nucleotide sequence represented by SEQ ID NOS: 137702, 137703, 137704, 137705, 137706, 137707, 137708, 137709, 137710, 137711, 137712, 137713, 137714, 137715, 137716, 137717, 137718, 137719, 137720, 137721, 137722, 137723 were selected from the ORFs having the nucleotide sequence represented by SEQ ID NO: 80246.

The probes having the nucleotide sequence represented by SEQ ID NOS: 137724, 137725, 137726, 137727, 137728, 137729, 137730, 137731, 137732, 137733, 137734 were selected from the ORFs having the nucleotide sequence represented by SEQ ID NO: 80247.

The probes having the nucleotide sequence represented by SEQ ID NOS: 137735, 137736, 137737, 137738, 137739, 137740, 137741, 137742, 137743, 137744, 137745 were selected from the ORFs having the nucleotide sequence represented by SEQ ID NO: 80248.

The probes having the nucleotide sequence represented by SEQ ID NOS: 137746, 137747, 137748, 137749, 137750, 137751, 137752, 137753, 137754, 137755, 137756 were selected from the ORFs having the nucleotide sequence represented by SEQ ID NO: 80249.

The probes having the nucleotide sequence represented by SEQ ID NOS: 137757, 137758, 137759, 137760, 137761, 137762, 137763, 137764, 137765, 137766, 137767 were selected from the ORFs having the nucleotide sequence represented by SEQ ID NO: 80250.

The probes having the nucleotide sequence represented by SEQ ID NOS: 137768, 137769, 137770, 137771, 137772, 137773, 137774, 137775, 137776, 137777, 137778 were selected from the ORFs having the nucleotide sequence represented by SEQ ID NO: 80251.

The probes having the nucleotide sequence represented by SEQ ID NOS: 137779, 137780, 137781, 137782, 137783, 137784, 137785, 137786, 137787, 137788, 137789 were selected from the ORFs having the nucleotide sequence represented by SEQ ID NO: 80252.

The probes having the nucleotide sequence represented by SEQ ID NOS: 137790, 137791, 137792, 137793, 137794, 137795, 137796, 137797, 137798, 137799, 137800 were selected from the ORFs having the nucleotide sequence represented by SEQ ID NO: 80253.

The probes having the nucleotide sequence represented by SEQ ID NOS: 137801, 137802, 137803, 137804, 137805, 137806, 137807, 137808, 137809, 137810, 137811 were selected from the ORFs having the nucleotide sequence represented by SEQ ID NO: 80254.

The probes having the nucleotide sequence represented by SEQ ID NOS: 137812, 137813, 137814, 137815, 137816, 137817, 137818, 137819, 137820, 137821, 137822 were selected from the ORFs having the nucleotide sequence represented by SEQ ID NO: 80255.

The probes having the nucleotide sequence represented by SEQ ID NOS: 137823, 137824, 137825, 137826, 137827, 137828, 137829, 137830, 137831, 137832, 137833 were selected from the ORFs having the nucleotide sequence represented by SEQ ID NO: 80256.

The probes having the nucleotide sequence represented by SEQ ID NOS: 137834, 137835, 137836, 137837, 137838, 137839, 137840, 137841, 137842, 137843, 137844 were selected from the ORFs having the nucleotide sequence represented by SEQ ID NO: 80257.

The probes having the nucleotide sequence represented by SEQ ID NOS: 137845, 137846, 137847, 137848, 137849, 137850, 137851, 137852, 137853, 137854, 137855 were selected from the ORFs having the nucleotide sequence represented by SEQ ID NO: 80258.

The probes having the nucleotide sequence represented by SEQ ID NOS: 137856, 137857, 137858, 137859, 137860, 137861, 137862, 137863, 137864, 137865, 137866 were selected from the ORFs having the nucleotide sequence represented by SEQ ID NO: 80259.

The probes having the nucleotide sequence represented by SEQ ID NOS: 137867, 137868, 137869, 137870, 137871, 137872, 137873, 137874, 137875, 137876, 137877 were selected from the ORFs having the nucleotide sequence represented by SEQ ID NO: 80260.

The probes having the nucleotide sequence represented by SEQ ID NOS: 137878, 137879, 137880, 137881, 137882, 137883, 137884, 137885, 137886, 137887, 137888 were selected from the ORFs having the nucleotide sequence represented by SEQ ID NO: 80261.

The probes having the nucleotide sequence represented by SEQ ID NOS: 137889, 137890, 137891, 137892, 137893, 137894, 137895, 137896, 137897, 137898, 137899 were selected from the ORFs having the nucleotide sequence represented by SEQ ID NO: 80262.

The probes having the nucleotide sequence represented by SEQ ID NOS: 137900, 137901, 137902, 137903, 137904, 137905, 137906, 137907, 137908, 137909, 137910 were selected from the ORFs having the nucleotide sequence represented by SEQ ID NO: 80263.

The probes having the nucleotide sequence represented by SEQ ID NOS: 137911, 137912, 137913, 137914, 137915, 137916, 137917, 137918, 137919, 137920, 137921, 137922, 137923, 137924, 137925, 137926, 137927, 137928, 137929, 137930, 137931, 137932 were selected from the ORFs having the nucleotide sequence represented by SEQ ID NO: 80264.

The probes having the nucleotide sequence represented by SEQ ID NOS: 137933, 137934, 137935, 137936, 137937, 137938, 137939, 137940, 137941, 137942, 137943 were selected from the ORFs having the nucleotide sequence represented by SEQ ID NO: 80265.

The probes having the nucleotide sequence represented by SEQ ID NOS: 137944, 137945, 137946, 137947, 137948, 137949, 137950, 137951, 137952, 137953, 137954 were selected from the ORFs having the nucleotide sequence represented by SEQ ID NO: 80266.

The probes having the nucleotide sequence represented by SEQ ID NOS: 137955, 137956, 137957, 137958, 137959, 137960, 137961, 137962, 137963, 137964, 137965 were selected from the ORFs having the nucleotide sequence represented by SEQ ID NO: 80267.

The probes having the nucleotide sequence represented by SEQ ID NOS: 137966, 137967, 137968, 137969, 137970, 137971, 137972, 137973, 137974, 137975, 137976 were selected from the ORFs having the nucleotide sequence represented by SEQ ID NO: 80268.

The probes having the nucleotide sequence represented by SEQ ID NOS: 137977, 137978, 137979, 137980, 137981, 137982, 137983, 137984, 137985, 137986, 137987 were selected from the ORFs having the nucleotide sequence represented by SEQ ID NO: 80269.

The probes having the nucleotide sequence represented by SEQ ID NOS: 137988, 137989, 137990, 137991, 137992, 137993, 137994, 137995, 137996, 137997, 137998 were selected from the ORFs having the nucleotide sequence represented by SEQ ID NO: 80270.

The probes having the nucleotide sequence represented by SEQ ID NOS: 137999, 138000, 138001, 138002, 138003, 138004, 138005, 138006, 138007, 138008, 138009 were selected from the ORFs having the nucleotide sequence represented by SEQ ID NO: 80271.

The probes having the nucleotide sequence represented by SEQ ID NOS: 138010, 138011, 138012, 138013, 138014, 138015, 138016, 138017, 138018, 138019, 138020 were selected from the ORFs having the nucleotide sequence represented by SEQ ID NO: 80272.

The probes having the nucleotide sequence represented by SEQ ID NOS: 138021, 138022, 138023, 138024, 138025, 138026, 138027, 138028, 138029, 138030, 138031 were selected from the ORFs having the nucleotide sequence represented by SEQ ID NO: 80273.

The probes having the nucleotide sequence represented by SEQ ID NOS: 138032, 138033, 138034, 138035, 138036, 138037, 138038, 138039, 138040, 138041, 138042 were selected from the ORFs having the nucleotide sequence represented by SEQ ID NO: 80274.

The probes having the nucleotide sequence represented by SEQ ID NOS: 138043, 138044, 138045, 138046, 138047, 138048, 138049, 138050, 138051, 138052, 138053 were selected from the ORFs having the nucleotide sequence represented by SEQ ID NO: 80275.

The probes having the nucleotide sequence represented by SEQ ID NOS: 138054, 138055, 138056, 138057, 138058, 138059, 138060, 138061, 138062, 138063, 138064 were selected from the ORFs having the nucleotide sequence represented by SEQ ID NO: 80276.

The probes having the nucleotide sequence represented by SEQ ID NOS: 138065, 138066, 138067, 138068, 138069, 138070, 138071, 138072, 138073, 138074, 138075 were selected from the ORFs having the nucleotide sequence represented by SEQ ID NO: 80277.

The probes having the nucleotide sequence represented by SEQ ID NOS: 138076, 138077, 138078, 138079, 138080, 138081, 138082, 138083, 138084, 138085, 138086 were selected from the ORFs having the nucleotide sequence represented by SEQ ID NO: 80278.

The probes having the nucleotide sequence represented by SEQ ID NOS: 138087, 138088, 138089, 138090, 138091, 138092, 138093, 138094, 138095, 138096, 138097 were selected from the ORFs having the nucleotide sequence represented by SEQ ID NO: 80279.

The probes having the nucleotide sequence represented by SEQ ID NOS: 138098, 138099, 138100, 138101, 138102, 138103, 138104, 138105, 138106, 138107, 138108 were selected from the ORFs having the nucleotide sequence represented by SEQ ID NO: 80280.

The probes having the nucleotide sequence represented by SEQ ID NOS: 138109, 138110, 138111, 138112, 138113, 138114, 138115, 138116, 138117, 138118, 138119 were selected from the ORFs having the nucleotide sequence represented by SEQ ID NO: 80281.

The probes having the nucleotide sequence represented by SEQ ID NOS: 138120, 138121, 138122, 138123, 138124, 138125, 138126, 138127, 138128, 138129, 138130 were selected from the ORFs having the nucleotide sequence represented by SEQ ID NO: 80282.

The probes having the nucleotide sequence represented by SEQ ID NOS: 138131, 138132, 138133, 138134, 138135, 138136, 138137, 138138, 138139, 138140, 138141 were selected from the ORFs having the nucleotide sequence represented by SEQ ID NO: 80283.

The probes having the nucleotide sequence represented by SEQ ID NOS: 138142, 138143, 138144, 138145, 138146, 138147, 138148, 138149, 138150, 138151, 138152 were selected from the ORFs having the nucleotide sequence represented by SEQ ID NO: 80284.

The probes having the nucleotide sequence represented by SEQ ID NOS: 138153, 138154, 138155, 138156, 138157, 138158, 138159, 138160, 138161, 138162, 138163 were selected from the ORFs having the nucleotide sequence represented by SEQ ID NO: 80285.

The probes having the nucleotide sequence represented by SEQ ID NOS: 138164, 138165, 138166, 138167, 138168, 138169, 138170, 138171, 138172, 138173, 138174 were selected from the ORFs having the nucleotide sequence represented by SEQ ID NO: 80286.

The probes having the nucleotide sequence represented by SEQ ID NOS: 138175, 138176, 138177, 138178, 138179, 138180, 138181, 138182, 138183, 138184, 138185 were selected from the ORFs having the nucleotide sequence represented by SEQ ID NO: 80287.

The probes having the nucleotide sequence represented by SEQ ID NOS: 138186, 138187, 138188, 138189, 138190, 138191, 138192, 138193, 138194, 138195, 138196 were selected from the ORFs having the nucleotide sequence represented by SEQ ID NO: 80288.

The probes having the nucleotide sequence represented by SEQ ID NOS: 138197, 138198, 138199, 138200, 138201, 138202, 138203, 138204, 138205, 138206, 138207 were selected from the ORFs having the nucleotide sequence represented by SEQ ID NO: 80289.

The probes having the nucleotide sequence represented by SEQ ID NOS: 138208, 138209, 138210, 138211, 138212, 138213, 138214, 138215, 138216, 138217, 138218 were selected from the ORFs having the nucleotide sequence represented by SEQ ID NO: 80290.

The probes having the nucleotide sequence represented by SEQ ID NOS: 138219, 138220, 138221, 138222, 138223, 138224, 138225, 138226, 138227, 138228, 138229 were selected from the ORFs having the nucleotide sequence represented by SEQ ID NO: 80291.

The probes having the nucleotide sequence represented by SEQ ID NOS: 138230, 138231, 138232, 138233, 138234, 138235, 138236, 138237, 138238, 138239, 138240 were selected from the ORFs having the nucleotide sequence represented by SEQ ID NO: 80292.

The probes having the nucleotide sequence represented by SEQ ID NOS: 138241, 138242, 138243, 138244, 138245, 138246, 138247, 138248, 138249, 138250, 138251 were selected from the ORFs having the nucleotide sequence represented by SEQ ID NO: 80293.

The probes having the nucleotide sequence represented by SEQ ID NOS: 138252, 138253, 138254, 138255, 138256, 138257, 138258, 138259, 138260, 138261, 138262 were selected from the ORFs having the nucleotide sequence represented by SEQ ID NO: 80294.

The probes having the nucleotide sequence represented by SEQ ID NOS: 138263, 138264, 138265, 138266, 138267, 138268, 138269, 138270, 138271, 138272, 138273 were selected from the ORFs having the nucleotide sequence represented by SEQ ID NO: 80295.

The probes having the nucleotide sequence represented by SEQ ID NOS: 138274, 138275, 138276, 138277, 138278, 138279, 138280, 138281, 138282, 138283, 138284 were selected from the ORFs having the nucleotide sequence represented by SEQ ID NO: 80296.

The probes having the nucleotide sequence represented by SEQ ID NOS: 138285, 138286, 138287, 138288, 138289, 138290, 138291, 138292, 138293, 138294, 138295 were selected from the ORFs having the nucleotide sequence represented by SEQ ID NO: 80297.

The probes having the nucleotide sequence represented by SEQ ID NOS: 138296, 138297, 138298, 138299, 138300, 138301, 138302, 138303, 138304, 138305, 138306 were selected from the ORFs having the nucleotide sequence represented by SEQ ID NO: 80298.

The probes having the nucleotide sequence represented by SEQ ID NOS: 138307, 138308, 138309, 138310, 138311, 138312, 138313, 138314, 138315, 138316, 138317 were selected from the ORFs having the nucleotide sequence represented by SEQ ID NO: 80299.

The probes having the nucleotide sequence represented by SEQ ID NOS: 138318, 138319, 138320, 138321, 138322, 138323, 138324, 138325, 138326, 138327, 138328 were selected from the ORFs having the nucleotide sequence represented by SEQ ID NO: 80300.

The probes having the nucleotide sequence represented by SEQ ID NOS: 138329, 138330, 138331, 138332, 138333, 138334, 138335, 138336, 138337, 138338, 138339 were selected from the ORFs having the nucleotide sequence represented by SEQ ID NO: 80301.

The probes having the nucleotide sequence represented by SEQ ID NOS: 138340, 138341, 138342, 138343, 138344, 138345, 138346, 138347, 138348, 138349, 138350 were selected from the ORFs having the nucleotide sequence represented by SEQ ID NO: 80302.

The probes having the nucleotide sequence represented by SEQ ID NOS: 138351, 138352, 138353, 138354, 138355, 138356, 138357, 138358, 138359, 138360, 138361 were selected from the ORFs having the nucleotide sequence represented by SEQ ID NO: 80303.

The probes having the nucleotide sequence represented by SEQ ID NOS: 138362, 138363, 138364, 138365, 138366, 138367, 138368, 138369, 138370, 138371, 138372 were selected from the ORFs having the nucleotide sequence represented by SEQ ID NO: 80304.

The probes having the nucleotide sequence represented by SEQ ID NOS: 138373, 138374, 138375, 138376, 138377, 138378, 138379, 138380, 138381, 138382, 138383 were selected from the ORFs having the nucleotide sequence represented by SEQ ID NO: 80305.

The probes having the nucleotide sequence represented by SEQ ID NOS: 138384, 138385, 138386, 138387, 138388, 138389, 138390, 138391, 138392, 138393, 138394 were selected from the ORFs having the nucleotide sequence represented by SEQ ID NO: 80306.

The probes having the nucleotide sequence represented by SEQ ID NOS: 138395, 138396, 138397, 138398, 138399, 138400, 138401, 138402, 138403, 138404, 138405 were selected from the ORFs having the nucleotide sequence represented by SEQ ID NO: 80307.

The probes having the nucleotide sequence represented by SEQ ID NOS: 138406, 138407, 138408, 138409, 138410, 138411, 138412, 138413, 138414, 138415, 138416 were selected from the ORFs having the nucleotide sequence represented by SEQ ID NO: 80308.

The probes having the nucleotide sequence represented by SEQ ID NOS: 138417, 138418, 138419, 138420, 138421, 138422, 138423, 138424, 138425, 138426, 138427 were selected from the ORFs having the nucleotide sequence represented by SEQ ID NO: 80309.

The probes having the nucleotide sequence represented by SEQ ID NOS: 138428, 138429, 138430, 138431, 138432, 138433, 138434, 138435, 138436, 138437, 138438 were selected from the ORFs having the nucleotide sequence represented by SEQ ID NO: 80310.

The probes having the nucleotide sequence represented by SEQ ID NOS: 138439, 138440, 138441, 138442, 138443, 138444, 138445, 138446, 138447, 138448, 138449 were selected from the ORFs having the nucleotide sequence represented by SEQ ID NO: 80311.

The probes having the nucleotide sequence represented by SEQ ID NOS: 138450, 138451, 138452, 138453, 138454, 138455, 138456, 138457, 138458, 138459, 138460 were selected from the ORFs having the nucleotide sequence represented by SEQ ID NO: 80312.

The probes having the nucleotide sequence represented by SEQ ID NOS: 138461, 138462, 138463, 138464, 138465, 138466, 138467, 138468, 138469, 138470, 138471 were selected from the ORFs having the nucleotide sequence represented by SEQ ID NO: 80313.

The probes having the nucleotide sequence represented by SEQ ID NOS: 138472, 138473, 138474, 138475, 138476, 138477, 138478, 138479, 138480, 138481, 138482 were selected from the ORFs having the nucleotide sequence represented by SEQ ID NO: 80314.

The probes having the nucleotide sequence represented by SEQ ID NOS: 138483, 138484, 138485, 138486, 138487, 138488, 138489, 138490, 138491, 138492, 138493 were selected from the ORFs having the nucleotide sequence represented by SEQ ID NO: 80315.

The probes having the nucleotide sequence represented by SEQ ID NOS: 138494, 138495, 138496, 138497, 138498, 138499, 138500, 138501, 138502, 138503, 138504 were selected from the ORFs having the nucleotide sequence represented by SEQ ID NO: 80316.

The probes having the nucleotide sequence represented by SEQ ID NOS: 138505, 138506, 138507, 138508, 138509, 138510, 138511, 138512, 138513, 138514, 138515 were selected from the ORFs having the nucleotide sequence represented by SEQ ID NO: 80317.

The probes having the nucleotide sequence represented by SEQ ID NOS: 138516, 138517, 138518, 138519, 138520, 138521, 138522, 138523, 138524, 138525, 138526 were selected from the ORFs having the nucleotide sequence represented by SEQ ID NO: 80318.

The probes having the nucleotide sequence represented by SEQ ID NOS: 138527, 138528, 138529, 138530, 138531, 138532, 138533, 138534, 138535, 138536, 138537 were selected from the ORFs having the nucleotide sequence represented by SEQ ID NO: 80319.

The probes having the nucleotide sequence represented by SEQ ID NOS: 138538, 138539, 138540, 138541, 138542, 138543, 138544, 138545, 138546, 138547, 138548 were selected from the ORFs having the nucleotide sequence represented by SEQ ID NO: 80320.

The probes having the nucleotide sequence represented by SEQ ID NOS: 138549, 138550, 138551, 138552, 138553, 138554, 138555, 138556, 138557, 138558, 138559 were selected from the ORFs having the nucleotide sequence represented by SEQ ID NO: 80321.

The probes having the nucleotide sequence represented by SEQ ID NOS: 138560, 138561, 138562, 138563, 138564, 138565, 138566, 138567, 138568, 138569, 138570 were selected from the ORFs having the nucleotide sequence represented by SEQ ID NO: 80322.

The probes having the nucleotide sequence represented by SEQ ID NOS: 138571, 138572, 138573, 138574, 138575, 138576, 138577, 138578, 138579, 138580, 138581 were selected from the ORFs having the nucleotide sequence represented by SEQ ID NO: 80323.

The probes having the nucleotide sequence represented by SEQ ID NOS: 138582, 138583, 138584, 138585, 138586, 138587, 138588, 138589, 138590, 138591, 138592 were selected from the ORFs having the nucleotide sequence represented by SEQ ID NO: 80324.

The probes having the nucleotide sequence represented by SEQ ID NOS: 138593, 138594, 138595, 138596, 138597, 138598, 138599, 138600, 138601, 138602, 138603 were selected from the ORFs having the nucleotide sequence represented by SEQ ID NO: 80325.

The probes having the nucleotide sequence represented by SEQ ID NOS: 138604, 138605, 138606, 138607, 138608, 138609, 138610, 138611, 138612, 138613, 138614 were selected from the ORFs having the nucleotide sequence represented by SEQ ID NO: 80326.

The probes having the nucleotide sequence represented by SEQ ID NOS: 138615, 138616, 138617, 138618, 138619, 138620, 138621, 138622, 138623, 138624, 138625 were selected from the ORFs having the nucleotide sequence represented by SEQ ID NO: 80327.

The probes having the nucleotide sequence represented by SEQ ID NOS: 138626, 138627, 138628, 138629, 138630, 138631, 138632, 138633, 138634, 138635, 138636 were selected from the ORFs having the nucleotide sequence represented by SEQ ID NO: 80328.

The probes having the nucleotide sequence represented by SEQ ID NOS: 138637, 138638, 138639, 138640, 138641, 138642, 138643, 138644, 138645, 138646, 138647 were selected from the ORFs having the nucleotide sequence represented by SEQ ID NO: 80329.

The probes having the nucleotide sequence represented by SEQ ID NOS: 138648, 138649, 138650, 138651, 138652, 138653, 138654, 138655, 138656, 138657, 138658 were selected from the ORFs having the nucleotide sequence represented by SEQ ID NO: 80330.

The probes having the nucleotide sequence represented by SEQ ID NOS: 138659, 138660, 138661, 138662, 138663, 138664, 138665, 138666, 138667, 138668, 138669 were selected from the ORFs having the nucleotide sequence represented by SEQ ID NO: 80331.

The probes having the nucleotide sequence represented by SEQ ID NOS: 138670, 138671, 138672, 138673, 138674, 138675, 138676, 138677, 138678, 138679, 138680 were selected from the ORFs having the nucleotide sequence represented by SEQ ID NO: 80332.

The probes having the nucleotide sequence represented by SEQ ID NOS: 138681, 138682, 138683, 138684, 138685, 138686, 138687, 138688, 138689, 138690, 138691 were selected from the ORFs having the nucleotide sequence represented by SEQ ID NO: 80333.

The probes having the nucleotide sequence represented by SEQ ID NOS: 138692, 138693, 138694, 138695, 138696, 138697, 138698, 138699, 138700, 138701, 138702 were selected from the ORFs having the nucleotide sequence represented by SEQ ID NO: 80334.

The probes having the nucleotide sequence represented by SEQ ID NOS: 138703, 138704, 138705, 138706, 138707, 138708, 138709, 138710, 138711, 138712, 138713 were selected from the ORFs having the nucleotide sequence represented by SEQ ID NO: 80335.

The probes having the nucleotide sequence represented by SEQ ID NOS: 138714, 138715, 138716, 138717, 138718, 138719, 138720, 138721, 138722, 138723, 138724 were selected from the ORFs having the nucleotide sequence represented by SEQ ID NO: 80336.

The probes having the nucleotide sequence represented by SEQ ID NOS: 138725, 138726, 138727, 138728, 138729, 138730, 138731, 138732, 138733, 138734, 138735 were selected from the ORFs having the nucleotide sequence represented by SEQ ID NO: 80337.

The probes having the nucleotide sequence represented by SEQ ID NOS: 138736, 138737, 138738, 138739, 138740, 138741, 138742, 138743, 138744, 138745, 138746 were selected from the ORFs having the nucleotide sequence represented by SEQ ID NO: 80338.

The probes having the nucleotide sequence represented by SEQ ID NOS: 138747, 138748, 138749, 138750, 138751, 138752, 138753, 138754, 138755, 138756, 138757 were selected from the ORFs having the nucleotide sequence represented by SEQ ID NO: 80339.

The probes having the nucleotide sequence represented by SEQ ID NOS: 138758, 138759, 138760, 138761, 138762, 138763, 138764, 138765, 138766, 138767, 138768 were selected from the ORFs having the nucleotide sequence represented by SEQ ID NO: 80340.

The probes having the nucleotide sequence represented by SEQ ID NOS: 138769, 138770, 138771, 138772, 138773, 138774, 138775, 138776, 138777, 138778, 138779, 138780, 138781, 138782, 138783, 138784, 138785, 138786, 138787, 138788, 138789, 138790 were selected from the ORFs having the nucleotide sequence represented by SEQ ID NO: 80341.

The probes having the nucleotide sequence represented by SEQ ID NOS: 138791, 138792, 138793, 138794, 138795, 138796, 138797, 138798, 138799, 138800, 138801 were selected from the ORFs having the nucleotide sequence represented by SEQ ID NO: 80342.

The probes having the nucleotide sequence represented by SEQ ID NOS: 138802, 138803, 138804, 138805, 138806, 138807, 138808, 138809, 138810, 138811, 138812 were selected from the ORFs having the nucleotide sequence represented by SEQ ID NO: 80343.

The probes having the nucleotide sequence represented by SEQ ID NOS: 138813, 138814, 138815, 138816, 138817, 138818, 138819, 138820, 138821, 138822, 138823 were selected from the ORFs having the nucleotide sequence represented by SEQ ID NO: 80344.

The probes having the nucleotide sequence represented by SEQ ID NOS: 138824, 138825, 138826, 138827, 138828, 138829, 138830, 138831, 138832, 138833, 138834 were selected from the ORFs having the nucleotide sequence represented by SEQ ID NO: 80345.

The probes having the nucleotide sequence represented by SEQ ID NOS: 138835, 138836, 138837, 138838, 138839, 138840, 138841, 138842, 138843, 138844, 138845 were selected from the ORFs having the nucleotide sequence represented by SEQ ID NO: 80346.

The probes having the nucleotide sequence represented by SEQ ID NOS: 138846, 138847, 138848, 138849, 138850, 138851, 138852, 138853, 138854, 138855, 138856 were selected from the ORFs having the nucleotide sequence represented by SEQ ID NO: 80347.

The probes having the nucleotide sequence represented by SEQ ID NOS: 138857, 138858, 138859, 138860, 138861, 138862, 138863, 138864, 138865, 138866, 138867 were selected from the ORFs having the nucleotide sequence represented by SEQ ID NO: 80348.

The probes having the nucleotide sequence represented by SEQ ID NOS: 138868, 138869, 138870, 138871, 138872, 138873, 138874, 138875, 138876, 138877, 138878 were selected from the ORFs having the nucleotide sequence represented by SEQ ID NO: 80349.

The probes having the nucleotide sequence represented by SEQ ID NOS: 138879, 138880, 138881, 138882, 138883, 138884, 138885, 138886, 138887, 138888, 138889 were selected from the ORFs having the nucleotide sequence represented by SEQ ID NO: 80350.

The probes having the nucleotide sequence represented by SEQ ID NOS: 138890, 138891, 138892, 138893, 138894, 138895, 138896, 138897, 138898, 138899, 138900 were selected from the ORFs having the nucleotide sequence represented by SEQ ID NO: 80351.

The probes having the nucleotide sequence represented by SEQ ID NOS: 138901, 138902, 138903, 138904, 138905, 138906, 138907, 138908, 138909, 138910, 138911 were selected from the ORFs having the nucleotide sequence represented by SEQ ID NO: 80352.

The probes having the nucleotide sequence represented by SEQ ID NOS: 138912, 138913, 138914, 138915, 138916, 138917, 138918, 138919, 138920, 138921, 138922 were selected from the ORFs having the nucleotide sequence represented by SEQ ID NO: 80353.

The probes having the nucleotide sequence represented by SEQ ID NOS: 138923, 138924, 138925, 138926, 138927, 138928, 138929, 138930, 138931, 138932, 138933 were selected from the ORFs having the nucleotide sequence represented by SEQ ID NO: 80354.

The probes having the nucleotide sequence represented by SEQ ID NOS: 138934, 138935, 138936, 138937, 138938, 138939, 138940, 138941, 138942, 138943, 138944 were selected from the ORFs having the nucleotide sequence represented by SEQ ID NO: 80355.

The probes having the nucleotide sequence represented by SEQ ID NOS: 138945, 138946, 138947, 138948, 138949, 138950, 138951, 138952, 138953, 138954, 138955 were selected from the ORFs having the nucleotide sequence represented by SEQ ID NO: 80356.

The probes having the nucleotide sequence represented by SEQ ID NOS: 138956, 138957, 138958, 138959, 138960, 138961, 138962, 138963, 138964, 138965, 138966 were selected from the ORFs having the nucleotide sequence represented by SEQ ID NO: 80357.

The probes having the nucleotide sequence represented by SEQ ID NOS: 138967, 138968, 138969, 138970, 138971, 138972, 138973, 138974, 138975, 138976, 138977 were selected from the ORFs having the nucleotide sequence represented by SEQ ID NO: 80358.

The probes having the nucleotide sequence represented by SEQ ID NOS: 138978, 138979, 138980, 138981, 138982, 138983, 138984, 138985, 138986, 138987, 138988 were selected from the ORFs having the nucleotide sequence represented by SEQ ID NO: 80359.

The probes having the nucleotide sequence represented by SEQ ID NOS: 138989, 138990, 138991, 138992, 138993, 138994, 138995, 138996, 138997, 138998, 138999 were selected from the ORFs having the nucleotide sequence represented by SEQ ID NO: 80360.

The probes having the nucleotide sequence represented by SEQ ID NOS: 139000, 139001, 139002, 139003, 139004, 139005, 139006, 139007, 139008, 139009, 139010 were selected from the ORFs having the nucleotide sequence represented by SEQ ID NO: 80361.

The probes having the nucleotide sequence represented by SEQ ID NOS: 139011, 139012, 139013, 139014, 139015, 139016, 139017, 139018, 139019, 139020, 139021 were selected from the ORFs having the nucleotide sequence represented by SEQ ID NO: 80362.

The probes having the nucleotide sequence represented by SEQ ID NOS: 139022, 139023, 139024, 139025, 139026, 139027, 139028, 139029, 139030, 139031, 139032 were selected from the ORFs having the nucleotide sequence represented by SEQ ID NO: 80363.

The probes having the nucleotide sequence represented by SEQ ID NOS: 139033, 139034, 139035, 139036, 139037, 139038, 139039, 139040, 139041, 139042, 139043 were selected from the ORFs having the nucleotide sequence represented by SEQ ID NO: 80364.

The probes having the nucleotide sequence represented by SEQ ID NOS: 139044, 139045, 139046, 139047, 139048, 139049, 139050, 139051, 139052, 139053, 139054 were selected from the ORFs having the nucleotide sequence represented by SEQ ID NO: 80365.

The probes having the nucleotide sequence represented by SEQ ID NOS: 139055, 139056, 139057, 139058, 139059, 139060, 139061, 139062, 139063, 139064, 139065 were selected from the ORFs having the nucleotide sequence represented by SEQ ID NO: 80366.

The probes having the nucleotide sequence represented by SEQ ID NOS: 139066, 139067, 139068, 139069, 139070, 139071, 139072, 139073, 139074, 139075, 139076 were selected from the ORFs having the nucleotide sequence represented by SEQ ID NO: 80367.

The probes having the nucleotide sequence represented by SEQ ID NOS: 139077, 139078, 139079, 139080, 139081, 139082, 139083, 139084, 139085, 139086, 139087 were selected from the ORFs having the nucleotide sequence represented by SEQ ID NO: 80368.

The probes having the nucleotide sequence represented by SEQ ID NOS: 139088, 139089, 139090, 139091, 139092, 139093, 139094, 139095, 139096, 139097, 139098 were selected from the ORFs having the nucleotide sequence represented by SEQ ID NO: 80369.

The probes having the nucleotide sequence represented by SEQ ID NOS: 139099, 139100, 139101, 139102, 139103, 139104, 139105, 139106, 139107, 139108, 139109 were selected from the ORFs having the nucleotide sequence represented by SEQ ID NO: 80370.

The probes having the nucleotide sequence represented by SEQ ID NOS: 139110, 139111, 139112, 139113, 139114, 139115, 139116, 139117, 139118, 139119, 139120 were selected from the ORFs having the nucleotide sequence represented by SEQ ID NO: 80371.

The probes having the nucleotide sequence represented by SEQ ID NOS: 139121, 139122, 139123, 139124, 139125, 139126, 139127, 139128, 139129, 139130, 139131 were selected from the ORFs having the nucleotide sequence represented by SEQ ID NO: 80372.

The probes having the nucleotide sequence represented by SEQ ID NOS: 139132, 139133, 139134, 139135, 139136, 139137, 139138, 139139, 139140, 139141, 139142 were selected from the ORFs having the nucleotide sequence represented by SEQ ID NO: 80373.

The probes having the nucleotide sequence represented by SEQ ID NOS: 139143, 139144, 139145, 139146, 139147, 139148, 139149, 139150, 139151, 139152, 139153 were selected from the ORFs having the nucleotide sequence represented by SEQ ID NO: 80374.

The probes having the nucleotide sequence represented by SEQ ID NOS: 139154, 139155, 139156, 139157, 139158, 139159, 139160, 139161, 139162, 139163, 139164 were selected from the ORFs having the nucleotide sequence represented by SEQ ID NO: 80375.

The probes having the nucleotide sequence represented by SEQ ID NOS: 139165, 139166, 139167, 139168, 139169, 139170, 139171, 139172, 139173, 139174, 139175 were selected from the ORFs having the nucleotide sequence represented by SEQ ID NO: 80376.

The probes having the nucleotide sequence represented by SEQ ID NOS: 139176, 139177, 139178, 139179, 139180, 139181, 139182, 139183, 139184, 139185, 139186 were selected from the ORFs having the nucleotide sequence represented by SEQ ID NO: 80377.

The probes having the nucleotide sequence represented by SEQ ID NOS: 139187, 139188, 139189, 139190, 139191, 139192, 139193, 139194, 139195, 139196, 139197 were selected from the ORFs having the nucleotide sequence represented by SEQ ID NO: 80378.

The probes having the nucleotide sequence represented by SEQ ID NOS: 139198, 139199, 139200, 139201, 139202, 139203, 139204, 139205, 139206, 139207, 139208 were selected from the ORFs having the nucleotide sequence represented by SEQ ID NO: 80379.

The probes having the nucleotide sequence represented by SEQ ID NOS: 139209, 139210, 139211, 139212, 139213, 139214, 139215, 139216, 139217, 139218, 139219 were selected from the ORFs having the nucleotide sequence represented by SEQ ID NO: 80380.

The probes having the nucleotide sequence represented by SEQ ID NOS: 139220, 139221, 139222, 139223, 139224, 139225, 139226, 139227, 139228, 139229, 139230 were selected from the ORFs having the nucleotide sequence represented by SEQ ID NO: 80381.

The probes having the nucleotide sequence represented by SEQ ID NOS: 139231, 139232, 139233, 139234, 139235, 139236, 139237, 139238, 139239, 139240, 139241 were selected from the ORFs having the nucleotide sequence represented by SEQ ID NO: 80382.

The probes having the nucleotide sequence represented by SEQ ID NOS: 139242, 139243, 139244, 139245, 139246, 139247, 139248, 139249, 139250, 139251, 139252 were selected from the ORFs having the nucleotide sequence represented by SEQ ID NO: 80383.

The probes having the nucleotide sequence represented by SEQ ID NOS: 139253, 139254, 139255, 139256, 139257, 139258, 139259, 139260, 139261, 139262, 139263 were selected from the ORFs having the nucleotide sequence represented by SEQ ID NO: 80384.

The probes having the nucleotide sequence represented by SEQ ID NOS: 139264, 139265, 139266, 139267, 139268, 139269, 139270, 139271, 139272, 139273, 139274 were selected from the ORFs having the nucleotide sequence represented by SEQ ID NO: 80385.

The probes having the nucleotide sequence represented by SEQ ID NOS: 139275, 139276, 139277, 139278, 139279, 139280, 139281, 139282, 139283, 139284, 139285 were selected from the ORFs having the nucleotide sequence represented by SEQ ID NO: 80386.

The probes having the nucleotide sequence represented by SEQ ID NOS: 139286, 139287, 139288, 139289, 139290, 139291, 139292, 139293, 139294, 139295, 139296 were selected from the ORFs having the nucleotide sequence represented by SEQ ID NO: 80387.

The probes having the nucleotide sequence represented by SEQ ID NOS: 139297, 139298, 139299, 139300, 139301, 139302, 139303, 139304, 139305, 139306, 139307, 139308, 139309, 139310, 139311, 139312, 139313, 139314, 139315, 139316, 139317, 139318 were selected from the ORFs having the nucleotide sequence represented by SEQ ID NO: 80388.

The probes having the nucleotide sequence represented by SEQ ID NOS: 139319, 139320, 139321, 139322, 139323, 139324, 139325, 139326, 139327, 139328, 139329 were selected from the ORFs having the nucleotide sequence represented by SEQ ID NO: 80389.

The probes having the nucleotide sequence represented by SEQ ID NOS: 139330, 139331, 139332, 139333, 139334, 139335, 139336, 139337, 139338, 139339, 139340 were selected from the ORFs having the nucleotide sequence represented by SEQ ID NO: 80390.

The probes having the nucleotide sequence represented by SEQ ID NOS: 139341, 139342, 139343, 139344, 139345, 139346, 139347, 139348, 139349, 139350, 139351 were selected from the ORFs having the nucleotide sequence represented by SEQ ID NO: 80391.

The probes having the nucleotide sequence represented by SEQ ID NOS: 139352, 139353, 139354, 139355, 139356, 139357, 139358, 139359, 139360, 139361, 139362 were selected from the ORFs having the nucleotide sequence represented by SEQ ID NO: 80392.

The probes having the nucleotide sequence represented by SEQ ID NOS: 139363, 139364, 139365, 139366, 139367, 139368, 139369, 139370, 139371, 139372, 139373 were selected from the ORFs having the nucleotide sequence represented by SEQ ID NO: 80393.

The probes having the nucleotide sequence represented by SEQ ID NOS: 139374, 139375, 139376, 139377, 139378, 139379, 139380, 139381, 139382, 139383, 139384 were selected from the ORFs having the nucleotide sequence represented by SEQ ID NO: 80394.

The probes having the nucleotide sequence represented by SEQ ID NOS: 139385, 139386, 139387, 139388, 139389, 139390, 139391, 139392, 139393, 139394, 139395 were selected from the ORFs having the nucleotide sequence represented by SEQ ID NO: 80395.

The probes having the nucleotide sequence represented by SEQ ID NOS: 139396, 139397, 139398, 139399, 139400, 139401, 139402, 139403, 139404, 139405, 139406 were selected from the ORFs having the nucleotide sequence represented by SEQ ID NO: 80396.

The probes having the nucleotide sequence represented by SEQ ID NOS: 139407, 139408, 139409, 139410, 139411, 139412, 139413, 139414, 139415, 139416, 139417 were selected from the ORFs having the nucleotide sequence represented by SEQ ID NO: 80397.

The probes having the nucleotide sequence represented by SEQ ID NOS: 139418, 139419, 139420, 139421, 139422, 139423, 139424, 139425, 139426, 139427, 139428 were selected from the ORFs having the nucleotide sequence represented by SEQ ID NO: 80398.

The probes having the nucleotide sequence represented by SEQ ID NOS: 139429, 139430, 139431, 139432, 139433, 139434, 139435, 139436, 139437, 139438, 139439 were selected from the ORFs having the nucleotide sequence represented by SEQ ID NO: 80399.

The probes having the nucleotide sequence represented by SEQ ID NOS: 139440, 139441, 139442, 139443, 139444, 139445, 139446, 139447, 139448, 139449, 139450 were selected from the ORFs having the nucleotide sequence represented by SEQ ID NO: 80400.

The probes having the nucleotide sequence represented by SEQ ID NOS: 139451, 139452, 139453, 139454, 139455, 139456, 139457, 139458, 139459, 139460, 139461 were selected from the ORFs having the nucleotide sequence represented by SEQ ID NO: 80401.

The probes having the nucleotide sequence represented by SEQ ID NOS: 139462, 139463, 139464, 139465, 139466, 139467, 139468, 139469, 139470, 139471, 139472 were selected from the ORFs having the nucleotide sequence represented by SEQ ID NO: 80402.

The probes having the nucleotide sequence represented by SEQ ID NOS: 139473, 139474, 139475, 139476, 139477, 139478, 139479, 139480, 139481, 139482, 139483 were selected from the ORFs having the nucleotide sequence represented by SEQ ID NO: 80403.

The probes having the nucleotide sequence represented by SEQ ID NOS: 139484, 139485, 139486, 139487, 139488, 139489, 139490, 139491, 139492, 139493, 139494 were selected from the ORFs having the nucleotide sequence represented by SEQ ID NO: 80404.

The probes having the nucleotide sequence represented by SEQ ID NOS: 139495, 139496, 139497, 139498, 139499, 139500, 139501, 139502, 139503, 139504, 139505 were selected from the ORFs having the nucleotide sequence represented by SEQ ID NO: 80405.

The probes having the nucleotide sequence represented by SEQ ID NOS: 139506, 139507, 139508, 139509, 139510, 139511, 139512, 139513, 139514, 139515, 139516 were selected from the ORFs having the nucleotide sequence represented by SEQ ID NO: 80406.

The probes having the nucleotide sequence represented by SEQ ID NOS: 139517, 139518, 139519, 139520, 139521, 139522, 139523, 139524, 139525, 139526, 139527 were selected from the ORFs having the nucleotide sequence represented by SEQ ID NO: 80407.

The probes having the nucleotide sequence represented by SEQ ID NOS: 139528, 139529, 139530, 139531, 139532, 139533, 139534, 139535, 139536, 139537, 139538 were selected from the ORFs having the nucleotide sequence represented by SEQ ID NO: 80408.

The probes having the nucleotide sequence represented by SEQ ID NOS: 139539, 139540, 139541, 139542, 139543, 139544, 139545, 139546, 139547, 139548, 139549 were selected from the ORFs having the nucleotide sequence represented by SEQ ID NO: 80409.

The probes having the nucleotide sequence represented by SEQ ID NOS: 139550, 139551, 139552, 139553, 139554, 139555, 139556, 139557, 139558, 139559, 139560 were selected from the ORFs having the nucleotide sequence represented by SEQ ID NO: 80410.

The probes having the nucleotide sequence represented by SEQ ID NOS: 139561, 139562, 139563, 139564, 139565, 139566, 139567, 139568, 139569, 139570, 139571 were selected from the ORFs having the nucleotide sequence represented by SEQ ID NO: 80411.

The probes having the nucleotide sequence represented by SEQ ID NOS: 139572, 139573, 139574, 139575, 139576, 139577, 139578, 139579, 139580, 139581, 139582 were selected from the ORFs having the nucleotide sequence represented by SEQ ID NO: 80412.

The probes having the nucleotide sequence represented by SEQ ID NOS: 139583, 139584, 139585, 139586, 139587, 139588, 139589, 139590, 139591, 139592, 139593 were selected from the ORFs having the nucleotide sequence represented by SEQ ID NO: 80413.

The probes having the nucleotide sequence represented by SEQ ID NOS: 139594, 139595, 139596, 139597, 139598, 139599, 139600, 139601, 139602, 139603, 139604 were selected from the ORFs having the nucleotide sequence represented by SEQ ID NO: 80414.

The probes having the nucleotide sequence represented by SEQ ID NOS: 139605, 139606, 139607, 139608, 139609, 139610, 139611, 139612, 139613, 139614, 139615 were selected from the ORFs having the nucleotide sequence represented by SEQ ID NO: 80415.

The probes having the nucleotide sequence represented by SEQ ID NOS: 139616, 139617, 139618, 139619, 139620, 139621, 139622, 139623, 139624, 139625, 139626 were selected from the ORFs having the nucleotide sequence represented by SEQ ID NO: 80416.

The probes having the nucleotide sequence represented by SEQ ID NOS: 139627, 139628, 139629, 139630, 139631, 139632, 139633, 139634, 139635, 139636, 139637 were selected from the ORFs having the nucleotide sequence represented by SEQ ID NO: 80417.

The probes having the nucleotide sequence represented by SEQ ID NOS: 139638, 139639, 139640, 139641, 139642, 139643, 139644, 139645, 139646, 139647, 139648 were selected from the ORFs having the nucleotide sequence represented by SEQ ID NO: 80418.

The probes having the nucleotide sequence represented by SEQ ID NOS: 139649, 139650, 139651, 139652, 139653, 139654, 139655, 139656, 139657, 139658, 139659 were selected from the ORFs having the nucleotide sequence represented by SEQ. ID NO: 80419.

The probes having the nucleotide sequence represented by SEQ ID NOS: 139660, 139661, 139662, 139663, 139664, 139665, 139666, 139667, 139668, 139669, 139670 were selected from the ORFs having the nucleotide sequence represented by SEQ ID NO: 80420.

The probes having the nucleotide sequence represented by SEQ ID NOS: 139671, 139672, 139673, 139674, 139675, 139676, 139677, 139678, 139679, 139680, 139681 were selected from the ORFs having the nucleotide sequence represented by SEQ ID NO: 80421.

The probes having the nucleotide sequence represented by SEQ ID NOS: 139682, 139683, 139684, 139685, 139686, 139687, 139688, 139689, 139690, 139691, 139692 were selected from the ORFs having the nucleotide sequence represented by SEQ ID NO: 80422.

The probes having the nucleotide sequence represented by SEQ ID NOS: 139693, 139694, 139695, 139696, 139697, 139698, 139699, 139700, 139701, 139702, 139703 were selected from the ORFs having the nucleotide sequence represented by SEQ ID NO: 80423.

The probes having the nucleotide sequence represented by SEQ ID NOS: 139704, 139705, 139706, 139707, 139708, 139709, 139710, 139711, 139712, 139713, 139714 were selected from the ORFs having the nucleotide sequence represented by SEQ ID NO: 80424.

The probes having the nucleotide sequence represented by SEQ ID NOS: 139715, 139716, 139717, 139718, 139719, 139720, 139721, 139722, 139723, 139724, 139725, 139726, 139727, 139728, 139729, 139730, 139731, 139732, 139733, 139734, 139735, 139736 were selected from the ORFs having the nucleotide sequence represented by SEQ ID NO: 80425.

The probes having the nucleotide sequence represented by SEQ ID NOS: 139737, 139738, 139739, 139740, 139741, 139742, 139743, 139744, 139745, 139746, 139747 were selected from the ORFs having the nucleotide sequence represented by SEQ ID NO: 80426.

The probes having the nucleotide sequence represented by SEQ ID NOS: 139748, 139749, 139750, 139751, 139752, 139753, 139754, 139755, 139756, 139757, 139758 were selected from the ORFs having the nucleotide sequence represented by SEQ ID NO: 80427.

The probes having the nucleotide sequence represented by SEQ ID NOS: 139759, 139760, 139761, 139762, 139763, 139764, 139765, 139766, 139767, 139768, 139769 were selected from the ORFs having the nucleotide sequence represented by SEQ ID NO: 80428.

The probes having the nucleotide sequence represented by SEQ ID NOS: 139770, 139771, 139772, 139773, 139774, 139775, 139776, 139777, 139778, 139779, 139780 were selected from the ORFs having the nucleotide sequence represented by SEQ ID NO: 80429.

The probes having the nucleotide sequence represented by SEQ ID NOS: 139781, 139782, 139783, 139784, 139785, 139786, 139787, 139788, 139789, 139790, 139791 were selected from the ORFs having the nucleotide sequence represented by SEQ ID NO: 80430.

The probes having the nucleotide sequence represented by SEQ ID NOS: 139792, 139793, 139794, 139795, 139796, 139797, 139798, 139799, 139800, 139801, 139802 were selected from the ORFs having the nucleotide sequence represented by SEQ ID NO: 80431.

The probes having the nucleotide sequence represented by SEQ ID NOS: 139803, 139804, 139805, 139806, 139807, 139808, 139809, 139810, 139811, 139812, 139813 were selected from the ORFs having the nucleotide sequence represented by SEQ ID NO: 80432.

The probes having the nucleotide sequence represented by SEQ ID NOS: 139814, 139815, 139816, 139817, 139818, 139819, 139820, 139821, 139822, 139823, 139824 were selected from the ORFs having the nucleotide sequence represented by SEQ ID NO: 80433.

The probes having the nucleotide sequence represented by SEQ ID NOS: 139825, 139826, 139827, 139828, 139829, 139830, 139831, 139832, 139833, 139834, 139835 were selected from the ORFs having the nucleotide sequence represented by SEQ ID NO: 80434.

The probes having the nucleotide sequence represented by SEQ ID NOS: 139836, 139837, 139838, 139839, 139840, 139841, 139842, 139843, 139844, 139845, 139846, 139847, 139848, 139849, 139850, 139851, 139852, 139853, 139854, 139855, 139856, 139857 were selected from the ORFs having the nucleotide sequence represented by SEQ ID NO: 80435.

The probes having the nucleotide sequence represented by SEQ ID NOS: 139858, 139859, 139860, 139861, 139862, 139863, 139864, 139865, 139866, 139867, 139868 were selected from the ORFs having the nucleotide sequence represented by SEQ ID NO: 80436.

The probes having the nucleotide sequence represented by SEQ ID NOS: 139869, 139870, 139871, 139872, 139873, 139874, 139875, 139876, 139877, 139878, 139879 were selected from the ORFs having the nucleotide sequence represented by SEQ ID NO: 80437.

The probes having the nucleotide sequence represented by SEQ ID NOS: 139880, 139881, 139882, 139883, 139884, 139885, 139886, 139887, 139888, 139889, 139890 were selected from the ORFs having the nucleotide sequence represented by SEQ ID NO: 80438.

The probes having the nucleotide sequence represented by SEQ ID NOS: 139891, 139892, 139893, 139894, 139895, 139896, 139897, 139898, 139899, 139900, 139901 were selected from the ORFs having the nucleotide sequence represented by SEQ ID NO: 80439.

The probes having the nucleotide sequence represented by SEQ ID NOS: 139902, 139903, 139904, 139905, 139906, 139907, 139908, 139909, 139910, 139911, 139912 were selected from the ORFs having the nucleotide sequence represented by SEQ ID NO: 80440.

The probes having the nucleotide sequence represented by SEQ ID NOS: 139913, 139914, 139915, 139916, 139917, 139918, 139919, 139920, 139921, 139922, 139923 were selected from the ORFs having the nucleotide sequence represented by SEQ ID NO: 80441.

The probes having the nucleotide sequence represented by SEQ ID NOS: 139924, 139925, 139926, 139927, 139928, 139929, 139930, 139931, 139932, 139933, 139934 were selected from the ORFs having the nucleotide sequence represented by SEQ ID NO: 80442.

The probes having the nucleotide sequence represented by SEQ ID NOS: 139935, 139936, 139937, 139938, 139939, 139940, 139941, 139942, 139943, 139944, 139945 were selected from the ORFs having the nucleotide sequence represented by SEQ ID NO: 80443.

The probes having the nucleotide sequence represented by SEQ ID NOS: 139946, 139947, 139948, 139949, 139950, 139951, 139952, 139953, 139954, 139955, 139956 were selected from the ORFs having the nucleotide sequence represented by SEQ ID NO: 80444.

The probes having the nucleotide sequence represented by SEQ ID NOS: 139957, 139958, 139959, 139960, 139961, 139962, 139963, 139964, 139965, 139966, 139967 were selected from the ORFs having the nucleotide sequence represented by SEQ ID NO: 80445.

The probes having the nucleotide sequence represented by SEQ ID NOS: 139968, 139969, 139970, 139971, 139972, 139973, 139974, 139975, 139976, 139977, 139978 were selected from the ORFs having the nucleotide sequence represented by SEQ ID NO: 80446.

The probes having the nucleotide sequence represented by SEQ ID NOS: 139979, 139980, 139981, 139982, 139983, 139984, 139985, 139986, 139987, 139988, 139989 were selected from the ORFs having the nucleotide sequence represented by SEQ ID NO: 80447.

The probes having the nucleotide sequence represented by SEQ ID NOS: 139990, 139991, 139992, 139993, 139994, 139995, 139996, 139997, 139998, 139999, 140000 were selected from the ORFs having the nucleotide sequence represented by SEQ ID NO: 80448.

The probes having the nucleotide sequence represented by SEQ ID NOS: 140001, 140002, 140003, 140004, 140005, 140006, 140007, 140008, 140009, 140010, 140011 were selected from the ORFs having the nucleotide sequence represented by SEQ ID NO: 80449.

The probes having the nucleotide sequence represented by SEQ ID NOS: 140012, 140013, 140014, 140015, 140016, 140017, 140018, 140019, 140020, 140021, 140022 were selected from the ORFs having the nucleotide sequence represented by SEQ ID NO: 80450.

The probes having the nucleotide sequence represented by SEQ ID NOS: 140023, 140024, 140025, 140026, 140027, 140028, 140029, 140030, 140031, 140032, 140033 were selected from the ORFs having the nucleotide sequence represented by SEQ ID NO: 80451.

The probes having the nucleotide sequence represented by SEQ ID NOS: 140034, 140035, 140036, 140037, 140038, 140039, 140040, 140041, 140042, 140043, 140044 were selected from the ORFs having the nucleotide sequence represented by SEQ ID NO: 80452.

The probes having the nucleotide sequence represented by SEQ ID NOS: 140045, 140046, 140047, 140048, 140049, 140050, 140051, 140052, 140053, 140054, 140055 were selected from the ORFs having the nucleotide sequence represented by SEQ ID NO: 80453.

The probes having the nucleotide sequence represented by SEQ ID NOS: 140056, 140057, 140058, 140059, 140060, 140061, 140062, 140063, 140064, 140065, 140066 were selected from the ORFs having the nucleotide sequence represented by SEQ ID NO: 80454.

The probes having the nucleotide sequence represented by SEQ ID NOS: 140067, 140068, 140069, 140070, 140071, 140072, 140073, 140074, 140075, 140076, 140077 were selected from the ORFs having the nucleotide sequence represented by SEQ ID NO: 80455.

The probes having the nucleotide sequence represented by SEQ ID NOS: 140078, 140079, 140080, 140081, 140082, 140083, 140084, 140085, 140086, 140087, 140088 were selected from the ORFs having the nucleotide sequence represented by SEQ ID NO: 80456.

The probes having the nucleotide sequence represented by SEQ ID NOS: 140089, 140090, 140091, 140092, 140093, 140094, 140095, 140096, 140097, 140098, 140099 were selected from the ORFs having the nucleotide sequence represented by SEQ ID NO: 80457

The probes having the nucleotide sequence represented by SEQ ID NOS: 140100, 140101, 140102, 140103, 140104, 140105, 140106, 140107, 140108, 140109, 140110 were selected from the ORFs having the nucleotide sequence represented by SEQ ID NO: 80458

The probes having the nucleotide sequence represented by SEQ ID NOS: 140111, 140112, 140113, 140114, 140115, 140116, 140117, 140118, 140119, 140120, 140121 were selected from the ORFs having the nucleotide sequence represented by SEQ ID NO: 80459

The probes having the nucleotide sequence represented by SEQ ID NOS: 140122, 140123, 140124, 140125, 140126, 140127, 140128, 140129, 140130, 140131, 140132 were selected from the ORFs having the nucleotide sequence represented by SEQ ID NO: 80460

The probes having the nucleotide sequence represented by SEQ ID NOS: 140133, 140134, 140135, 140136, 140137, 140138, 140139, 140140, 140141, 140142, 140143 were selected from the ORFs having the nucleotide sequence represented by SEQ ID NO: 80461

The probes having the nucleotide sequence represented by SEQ ID NOS: 140144, 140145, 140146, 140147, 140148, 140149, 140150, 140151, 140152, 140153, 140154 were selected from the ORFs having the nucleotide sequence represented by SEQ ID NO: 80462.

The probes having the nucleotide sequence represented by SEQ ID NOS: 140155, 140156, 140157, 140158, 140159, 140160, 140161, 140162, 140163, 140164, 140165 were selected from the ORFs having the nucleotide sequence represented by SEQ ID NO: 80463

The probes having the nucleotide sequence represented by SEQ ID NOS: 140166, 140167, 140168, 140169, 140170, 140171, 140172, 140173, 140174, 140175, 140176 were selected from the ORFs having the nucleotide sequence represented by SEQ ID NO: 80464

The probes having the nucleotide sequence represented by SEQ ID NOS: 140177, 140178, 140179, 140180, 140181, 140182, 140183, 140184, 140185, 140186, 140187 were selected from the ORFs having the nucleotide sequence represented by SEQ ID NO: 80465

The probes having the nucleotide sequence represented by SEQ ID NOS: 140188, 140189, 140190, 140191, 140192, 140193, 140194, 140195, 140196, 140197, 140198 were selected from the ORFs having the nucleotide sequence represented by SEQ ID NO: 80466

The probes having the nucleotide sequence represented by SEQ ID NOS: 140199, 140200, 140201, 140202, 140203, 140204, 140205, 140206, 140207, 140208, 140209 were selected from the ORFs having the nucleotide sequence represented by SEQ ID NO: 80467

The probes having the nucleotide sequence represented by SEQ ID NOS: 140210, 140211, 140212, 140213, 140214, 140215, 140216, 140217, 140218, 140219, 140220 were selected from the ORFs having the nucleotide sequence represented by SEQ ID NO: 80468

The probes having the nucleotide sequence represented by SEQ ID NOS: 140221, 140222, 140223, 140224, 140225, 140226, 140227, 140228, 140229, 140230, 140231 were selected from the ORFs having the nucleotide sequence represented by SEQ ID NO: 80469

The probes having the nucleotide sequence represented by SEQ ID NOS: 140232, 140233, 140234, 140235, 140236, 140237, 140238, 140239, 140240, 140241, 140242 were selected from the ORFs having the nucleotide sequence represented by SEQ ID NO: 80470

The probes having the nucleotide sequence represented by SEQ ID NOS: 140243, 140244, 140245, 140246, 140247, 140248, 140249, 140250, 140251, 140252, 140253 were selected from the ORFs having the nucleotide sequence represented by SEQ ID NO: 80471

The probes having the nucleotide sequence represented by SEQ ID NOS: 140254, 140255, 140256, 140257, 140258, 140259, 140260, 140261, 140262, 140263, 140264 were selected from the ORFs having the nucleotide sequence represented by SEQ ID NO: 80472

The probes having the nucleotide sequence represented by SEQ ID NOS: 140265, 140266, 140267, 140268, 140269, 140270, 140271, 140272, 140273, 140274, 140275 were selected from the ORFs having the nucleotide sequence represented by SEQ ID NO: 80473.

The probes having the nucleotide sequence represented by SEQ ID NOS: 140276, 140277, 140278, 140279, 140280, 140281, 140282, 140283, 140284, 140285, 140286 were selected from the ORFs having the nucleotide sequence represented by SEQ ID NO: 80474

The probes having the nucleotide sequence represented by SEQ ID NOS: 140287, 140288, 140289, 140290, 140291, 140292, 140293, 140294, 140295, 140296, 140297 were selected from the ORFs having the nucleotide sequence represented by SEQ ID NO: 80475

The probes having the nucleotide sequence represented by SEQ ID NOS: 140298, 140299, 140300, 140301, 140302, 140303, 140304, 140305, 140306, 140307, 140308 were selected from the ORFs having the nucleotide sequence represented by SEQ ID NO: 80476

The probes having the nucleotide sequence represented by SEQ ID NOS: 140309, 140310, 140311, 140312, 140313, 140314, 140315, 140316, 140317, 140318, 140319 were selected from the ORFs having the nucleotide sequence represented by SEQ ID NO: 80477

The probes having the nucleotide sequence represented by SEQ ID NOS: 140320, 140321, 140322, 140323, 140324, 140325, 140326, 140327, 140328, 140329, 140330 were selected from the ORFs having the nucleotide sequence represented by SEQ ID NO: 80478

The probes having the nucleotide sequence represented by SEQ ID NOS: 140331, 140332, 140333, 140334, 140335, 140336, 140337, 140338, 140339, 140340, 140341 were selected from the ORFs having the nucleotide sequence represented by SEQ ID NO: 80479

The probes having the nucleotide sequence represented by SEQ ID NOS: 140342, 140343, 140344, 140345, 140346, 140347, 140348, 140349, 140350, 140351, 140352 were selected from the ORFs having the nucleotide sequence represented by SEQ ID NO: 80480

The probes having the nucleotide sequence represented by SEQ ID NOS: 140353, 140354, 140355, 140356, 140357, 140358, 140359, 140360, 140361, 140362, 140363 were selected from the ORFs having the nucleotide sequence represented by SEQ ID NO: 80481

The probes having the nucleotide sequence represented by SEQ ID NOS: 140364, 140365, 140366, 140367, 140368, 140369, 140370, 140371, 140372, 140373, 140374 were selected from the ORFs having the nucleotide sequence represented by SEQ ID NO: 80482

The probes having the nucleotide sequence represented by SEQ ID NOS: 140375, 140376, 140377, 140378, 140379, 140380, 140381, 140382, 140383, 140384, 140385 were selected from the ORFs having the nucleotide sequence represented by SEQ ID NO: 80483

The probes having the nucleotide sequence represented by SEQ ID NOS: 140386, 140387, 140388, 140389, 140390, 140391, 140392, 140393, 140394, 140395, 140396 were selected from the ORFs having the nucleotide sequence represented by SEQ ID NO: 80484.

The probes having the nucleotide sequence represented by SEQ ID NOS: 140397, 140398, 140399, 140400, 140401, 140402, 140403, 140404, 140405, 140406, 140407 were selected from the ORFs having the nucleotide sequence represented by SEQ ID NO: 80485

The probes having the nucleotide sequence represented by SEQ ID NOS: 140408, 140409, 140410, 140411, 140412, 140413, 140414, 140415, 140416, 140417, 140418 were selected from the ORFs having the nucleotide sequence represented by SEQ ID NO: 80486

The probes having the nucleotide sequence represented by SEQ ID NOS: 140419, 140420, 140421, 140422, 140423, 140424, 140425, 140426, 140427, 140428, 140429 were selected from the ORFs having the nucleotide sequence represented by SEQ ID NO: 80487

The probes having the nucleotide sequence represented by SEQ ID NOS: 140430, 140431, 140432, 140433, 140434, 140435, 140436, 140437, 140438, 140439, 140440 were selected from the ORFs having the nucleotide sequence represented by SEQ ID NO: 80488

The probes having the nucleotide sequence represented by SEQ ID NOS: 140441, 140442, 140443, 140444, 140445, 140446, 140447, 140448, 140449, 140450, 140451 were selected from the ORFs having the nucleotide sequence represented by SEQ ID NO: 80489

The probes having the nucleotide sequence represented by SEQ ID NOS: 140452, 140453, 140454, 140455, 140456, 140457, 140458, 140459, 140460, 140461, 140462 were selected from the ORFs having the nucleotide sequence represented by SEQ ID NO: 80490

The probes having the nucleotide sequence represented by SEQ ID NOS: 140463, 140464, 140465, 140466, 140467, 140468, 140469, 140470, 140471, 140472, 140473 were selected from the ORFs having the nucleotide sequence represented by SEQ ID NO: 80491

The probes having the nucleotide sequence represented by SEQ ID NOS: 140474, 140475, 140476, 140477, 140478, 140479, 140480, 140481, 140482, 140483, 140484 were selected from the ORFs having the nucleotide sequence represented by SEQ ID NO: 80492

The probes having the nucleotide sequence represented by SEQ ID NOS: 140485, 140486, 140487, 140488, 140489, 140490, 140491, 140492, 140493, 140494, 140495 were selected from the ORFs having the nucleotide sequence represented by SEQ ID NO: 80493

The probes having the nucleotide sequence represented by SEQ ID NOS: 140496, 140497, 140498, 140499, 140500, 140501, 140502, 140503, 140504, 140505, 140506 were selected from the ORFs having the nucleotide sequence represented by SEQ ID NO: 80494

The probes having the nucleotide sequence represented by SEQ ID NOS: 140507, 140508, 140509, 140510, 140511, 140512, 140513, 140514, 140515, 140516, 140517 were selected from the ORFs having the nucleotide sequence represented by SEQ ID NO: 80495.

The probes having the nucleotide sequence represented by SEQ ID NOS: 140518, 140519, 140520, 140521, 140522, 140523, 140524, 140525, 140526, 140527, 140528 were selected from the ORFs having the nucleotide sequence represented by SEQ ID NO: 80496

The probes having the nucleotide sequence represented by SEQ ID NOS: 140529, 140530, 140531, 140532, 140533, 140534, 140535, 140536, 140537, 140538, 140539 were selected from the ORFs having the nucleotide sequence represented by SEQ ID NO: 80497

The probes having the nucleotide sequence represented by SEQ ID NOS: 140540, 140541, 140542, 140543, 140544, 140545, 140546, 140547, 140548, 140549, 140550 were selected from the ORFs having the nucleotide sequence represented by SEQ ID NO: 80498

The probes having the nucleotide sequence represented by SEQ ID NOS: 140551, 140552, 140553, 140554, 140555, 140556, 140557, 140558, 140559, 140560, 140561 were selected from the ORFs having the nucleotide sequence represented by SEQ ID NO: 80499

The probes having the nucleotide sequence represented by SEQ ID NOS: 140562, 140563, 140564, 140565, 140566, 140567, 140568, 140569, 140570, 140571, 140572 were selected from the ORFs having the nucleotide sequence represented by SEQ ID NO: 80500

The probes having the nucleotide sequence represented by SEQ ID NOS: 140573, 140574, 140575, 140576, 140577, 140578, 140579, 140580, 140581, 140582, 140583 were selected from the ORFs having the nucleotide sequence represented by SEQ ID NO: 80501

The probes having the nucleotide sequence represented by SEQ ID NOS: 140584, 140585, 140586, 140587, 140588, 140589, 140590, 140591, 140592, 140593, 140594 were selected from the ORFs having the nucleotide sequence represented by SEQ ID NO: 80502

The probes having the nucleotide sequence represented by SEQ ID NOS: 140595, 140596, 140597, 140598, 140599, 140600, 140601, 140602, 140603, 140604, 140605 were selected from the ORFs having the nucleotide sequence represented by SEQ ID NO: 80503

The probes having the nucleotide sequence represented by SEQ ID NOS: 140606, 140607, 140608, 140609, 140610, 140611, 140612, 140613, 140614, 140615, 140616 were selected from the ORFs having the nucleotide sequence represented by SEQ ID NO: 80504

The probes having the nucleotide sequence represented by SEQ ID NOS: 140617, 140618, 140619, 140620, 140621, 140622, 140623, 140624, 140625, 140626, 140627 were selected from the ORFs having the nucleotide sequence represented by SEQ ID NO: 80505

The probes having the nucleotide sequence represented by SEQ ID NOS: 140628, 140629, 140630, 140631, 140632, 140633, 140634, 140635, 140636, 140637, 140638 were selected from the ORFs having the nucleotide sequence represented by SEQ ID NO: 80506.

The probes having the nucleotide sequence represented by SEQ ID NOS: 140639, 140640, 140641, 140642, 140643, 140644, 140645, 140646, 140647, 140648, 140649 were selected from the ORFs having the nucleotide sequence represented by SEQ ID NO: 80507

The probes having the nucleotide sequence represented by SEQ ID NOS: 140650, 140651, 140652, 140653, 140654, 140655, 140656, 140657, 140658, 140659, 140660 were selected from the ORFs having the nucleotide sequence represented by SEQ ID NO: 80508

The probes having the nucleotide sequence represented by SEQ ID NOS: 140661, 140662, 140663, 140664, 140665, 140666, 140667, 140668, 140669, 140670, 140671 were selected from the ORFs having the nucleotide sequence represented by SEQ ID NO: 80509

The probes having the nucleotide sequence represented by SEQ ID NOS: 140672, 140673, 140674, 140675, 140676, 140677, 140678, 140679, 140680, 140681, 140682 were selected from the ORFs having the nucleotide sequence represented by SEQ ID NO: 80510

The probes having the nucleotide sequence represented by SEQ ID NOS: 140683, 140684, 140685, 140686, 140687, 140688, 140689, 140690, 140691, 140692, 140693 were selected from the ORFs having the nucleotide sequence represented by SEQ ID NO: 80511

The probes having the nucleotide sequence represented by SEQ ID NOS: 140694, 140695, 140696, 140697, 140698, 140699, 140700, 140701, 140702, 140703, 140704 were selected from the ORFs having the nucleotide sequence represented by SEQ ID NO: 80512

The probes having the nucleotide sequence represented by SEQ ID NOS: 140705, 140706, 140707, 140708, 140709, 140710, 140711, 140712, 140713, 140714, 140715 were selected from the ORFs having the nucleotide sequence represented by SEQ ID NO: 80513

The probes having the nucleotide sequence represented by SEQ ID NOS: 140716, 140717, 140718, 140719, 140720, 140721, 140722, 140723, 140724, 140725, 140726 were selected from the ORFs having the nucleotide sequence represented by SEQ ID NO: 80514

The probes having the nucleotide sequence represented by SEQ ID NOS: 140727, 140728, 140729, 140730, 140731, 140732, 140733, 140734, 140735, 140736, 140737 were selected from the ORFs having the nucleotide sequence represented by SEQ ID NO: 80515

The probes having the nucleotide sequence represented by SEQ ID NOS: 140738, 140739, 140740, 140741, 140742, 140743, 140744, 140745, 140746, 140747, 140748 were selected from the ORFs having the nucleotide sequence represented by SEQ ID NO: 80516.

The probes having the nucleotide sequence represented by SEQ ID NOS: 140749, 140750, 140751, 140752, 140753, 140754, 140755, 140756, 140757, 140758, 140759 were selected from the ORFs having the nucleotide sequence represented by SEQ ID NO: 80517.

The probes having the nucleotide sequence represented by SEQ ID NOS: 140760, 140761, 140762, 140763, 140764, 140765, 140766, 140767, 140768, 140769, 140770 were selected from the ORFs having the nucleotide sequence represented by SEQ ID NO: 80518

The probes having the nucleotide sequence represented by SEQ ID NOS: 140771, 140772, 140773, 140774, 140775, 140776, 140777, 140778, 140779, 140780, 140781 were selected from the ORFs having the nucleotide sequence represented by SEQ ID NO: 80519

The probes having the nucleotide sequence represented by SEQ ID NOS: 140782, 140783, 140784, 140785, 140786, 140787, 140788, 140789, 140790, 140791, 140792 were selected from the ORFs having the nucleotide sequence represented by SEQ ID NO: 80520

The probes having the nucleotide sequence represented by SEQ ID NOS: 140793, 140794, 140795, 140796, 140797, 140798, 140799, 140800, 140801, 140802, 140803 were selected from the ORFs having the nucleotide sequence represented by SEQ ID NO: 80521

The probes having the nucleotide sequence represented by SEQ ID NOS: 140804, 140805, 140806, 140807, 140808, 140809, 140810, 140811, 140812, 140813, 140814 were selected from the ORFs having the nucleotide sequence represented by SEQ ID NO: 80522

The probes having the nucleotide sequence represented by SEQ ID NOS: 140815, 140816, 140817, 140818, 140819, 140820, 140821, 140822, 140823, 140824, 140825 were selected from the ORFs having the nucleotide sequence represented by SEQ ID NO: 80523

The probes having the nucleotide sequence represented by SEQ ID NOS: 140826, 140827, 140828, 140829, 140830, 140831, 140832, 140833, 140834, 140835, 140836 were selected from the ORFs having the nucleotide sequence represented by SEQ ID NO: 80524

The probes having the nucleotide sequence represented by SEQ ID NOS: 140837, 140838, 140839, 140840, 140841, 140842, 140843, 140844, 140845, 140846, 140847 were selected from the ORFs having the nucleotide sequence represented by SEQ ID NO: 80525

The probes having the nucleotide sequence represented by SEQ ID NOS: 140848, 140849, 140850, 140851, 140852, 140853, 140854, 140855, 140856, 140857, 140858 were selected from the ORFs having the nucleotide sequence represented by SEQ ID NO: 80526

The probes having the nucleotide sequence represented by SEQ ID NOS: 140859, 140860, 140861, 140862, 140863, 140864, 140865, 140866, 140867, 140868, 140869 were selected from the ORFs having the nucleotide sequence represented by SEQ ID NO: 80527

The probes having the nucleotide sequence represented by SEQ ID NOS: 140870, 140871, 140872, 140873, 140874, 140875, 140876, 140877, 140878, 140879, 140880 were selected from the ORFs having the nucleotide sequence represented by SEQ ID NO: 80528.

The probes having the nucleotide sequence represented by SEQ ID NOS: 140881, 140882, 140883, 140884, 140885, 140886, 140887, 140888, 140889, 140890, 140891 were selected from the ORFs having the nucleotide sequence represented by SEQ ID NO: 80529

The probes having the nucleotide sequence represented by SEQ ID NOS: 140892, 140893, 140894, 140895, 140896, 140897, 140898, 140899, 140900, 140901, 140902 were selected from the ORFs having the nucleotide sequence represented by SEQ ID NO: 80530

The probes having the nucleotide sequence represented by SEQ ID NOS: 140903, 140904, 140905, 140906, 140907, 140908, 140909, 140910, 140911, 140912, 140913 were selected from the ORFs having the nucleotide sequence represented by SEQ ID NO: 80531

The probes having the nucleotide sequence represented by SEQ ID NOS: 140914, 140915, 140916, 140917, 140918, 140919, 140920, 140921, 140922, 140923, 140924 were selected from the ORFs having the nucleotide sequence represented by SEQ ID NO: 80532

The probes having the nucleotide sequence represented by SEQ ID NOS: 140925, 140926, 140927, 140928, 140929, 140930, 140931, 140932, 140933, 140934, 140935 were selected from the ORFs having the nucleotide sequence represented by SEQ ID NO: 80533

The probes having the nucleotide sequence represented by SEQ ID NOS: 140936, 140937, 140938, 140939, 140940, 140941, 140942, 140943, 140944, 140945, 140946 were selected from the ORFs having the nucleotide sequence represented by SEQ ID NO: 80534

The probes having the nucleotide sequence represented by SEQ ID NOS: 140947, 140948, 140949, 140950, 140951, 140952, 140953, 140954, 140955, 140956, 140957 were selected from the ORFs having the nucleotide sequence represented by SEQ ID NO: 80535

The probes having the nucleotide sequence represented by SEQ ID NOS: 140958, 140959, 140960, 140961, 140962, 140963, 140964, 140965, 140966, 140967, 140968 were selected from the ORFs having the nucleotide sequence represented by SEQ ID NO: 80536

The probes having the nucleotide sequence represented by SEQ ID NOS: 140969, 140970, 140971, 140972, 140973, 140974, 140975, 140976, 140977, 140978, 140979 were selected from the ORFs having the nucleotide sequence represented by SEQ ID NO: 80537

The probes having the nucleotide sequence represented by SEQ ID NOS: 140980, 140981, 140982, 140983, 140984, 140985, 140986, 140987, 140988, 140989, 140990 were selected from the ORFs having the nucleotide sequence represented by SEQ ID NO: 80538

The probes having the nucleotide sequence represented by SEQ ID NOS: 140991, 140992, 140993, 140994, 140995, 140996, 140997, 140998, 140999, 141000, 141001 were selected from the ORFs having the nucleotide sequence represented by SEQ ID NO: 80539.

The probes having the nucleotide sequence represented by SEQ ID NOS: 141002, 141003, 141004, 141005, 141006, 141007, 141008, 141009, 141010, 141011, 141012 were selected from the ORFs having the nucleotide sequence represented by SEQ ID NO: 80540

The probes having the nucleotide sequence represented by SEQ ID NOS: 141013, 141014, 141015, 141016, 141017, 141018, 141019, 141020, 141021, 141022, 141023 were selected from the ORFs having the nucleotide sequence represented by SEQ ID NO: 80541

The probes having the nucleotide sequence represented by SEQ ID NOS: 141024, 141025, 141026, 141027, 141028, 141029, 141030, 141031, 141032, 141033, 141034 were selected from the ORFs having the nucleotide sequence represented by SEQ ID NO: 80542

The probes having the nucleotide sequence represented by SEQ ID NOS: 141035, 141036, 141037, 141038, 141039, 141040, 141041, 141042, 141043, 141044, 141045 were selected from the ORFs having the nucleotide sequence represented by SEQ ID NO: 80543

The probes having the nucleotide sequence represented by SEQ ID NOS: 141046, 141047, 141048, 141049, 141050, 141051, 141052, 141053, 141054, 141055, 141056 were selected from the ORFs having the nucleotide sequence represented by SEQ ID NO: 80544

The probes having the nucleotide sequence represented by SEQ ID NOS: 141057, 141058, 141059, 141060, 141061, 141062, 141063, 141064, 141065, 141066, 141067 were selected from the ORFs having the nucleotide sequence represented by SEQ ID NO: 80545

The probes having the nucleotide sequence represented by SEQ ID NOS: 141068, 141069, 141070, 141071, 141072, 141073, 141074, 141075, 141076, 141077, 141078 were selected from the ORFs having the nucleotide sequence represented by SEQ ID NO: 80546

The probes having the nucleotide sequence represented by SEQ ID NOS: 141079, 141080, 141081, 141082, 141083, 141084, 141085, 141086, 141087, 141088, 141089 were selected from the ORFs having the nucleotide sequence represented by SEQ ID NO: 80547

The probes having the nucleotide sequence represented by SEQ ID NOS: 141090, 141091, 141092, 141093, 141094, 141095, 141096, 141097, 141098, 141099, 141100 were selected from the ORFs having the nucleotide sequence represented by SEQ ID NO: 80548

The probes having the nucleotide sequence represented by SEQ ID NOS: 141101, 141102, 141103, 141104, 141105, 141106, 141107, 141108, 141109, 141110, 141111 were selected from the ORFs having the nucleotide sequence represented by SEQ ID NO: 80549

The probes having the nucleotide sequence represented by SEQ ID NOS: 141112, 141113, 141114, 141115, 141116, 141117, 141118, 141119, 141120, 141121, 141122 were selected from the ORFs having the nucleotide sequence represented by SEQ ID NO: 80550.

The probes having the nucleotide sequence represented by SEQ ID NOS: 141123, 141124, 141125, 141126, 141127, 141128, 141129, 141130, 141131, 141132, 141133 were selected from the ORFs having the nucleotide sequence represented by SEQ ID NO: 80551

The probes having the nucleotide sequence represented by SEQ ID NOS: 141134, 141135, 141136, 141137, 141138, 141139, 141140, 141141, 141142, 141143, 141144 were selected from the ORFs having the nucleotide sequence represented by SEQ ID NO: 80552

The probes having the nucleotide sequence represented by SEQ ID NOS: 141145, 141146, 141147, 141148, 141149, 141150, 141151, 141152, 141153, 141154, 141155 were selected from the ORFs having the nucleotide sequence represented by SEQ ID NO: 80553

The probes having the nucleotide sequence represented by SEQ ID NOS: 141156, 141157, 141158, 141159, 141160, 141161, 141162, 141163, 141164, 141165, 141166 were selected from the ORFs having the nucleotide sequence represented by SEQ ID NO: 80554

The probes having the nucleotide sequence represented by SEQ ID NOS: 141167, 141168, 141169, 141170, 141171, 141172, 141173, 141174, 141175, 141176, 141177 were selected from the ORFs having the nucleotide sequence represented by SEQ ID NO: 80555

The probes having the nucleotide sequence represented by SEQ ID NOS: 141178, 141179, 141180, 141181, 141182, 141183, 141184, 141185, 141186, 141187, 141188 were selected from the ORFs having the nucleotide sequence represented by SEQ. ID NO: 80556

The probes having the nucleotide sequence represented by. SEQ ID NOS: 141189, 141190, 141191, 141192, 141193, 141194, 141195, 141196, 141197, 141198, 141199 were selected from the ORFs having the nucleotide sequence represented by SEQ ID NO: 80557

The probes having the nucleotide sequence represented by SEQ ID NOS: 141200, 141201, 141202, 141203, 141204, 141205, 141206, 141207, 141208, 141209, 141210 were selected from the ORFs having the nucleotide sequence represented by SEQ ID NO: 80558

The probes having the nucleotide sequence represented by SEQ ID NOS: 141211, 141212, 141213, 141214, 141215, 141216, 141217, 141218, 141219, 141220, 141221 were selected from the ORFs having the nucleotide sequence represented by SEQ ID NO: 80559

The probes having the nucleotide sequence represented by SEQ ID NOS: 141222, 141223, 141224, 141225, 141226, 141227, 141228, 141229, 141230, 141231, 141232 were selected from the ORFs having the nucleotide sequence represented by SEQ ID NO: 80560

The probes having the nucleotide sequence represented by SEQ ID NOS: 141233, 141234, 141235, 141236, 141237, 141238, 141239, 141240, 141241, 141242, 141243, 141244, 141245, 141246, 141247, 141248, 141249, 141250, 141251, 141252, 141253, 141254 were selected from the ORFs having the nucleotide sequence represented by SEQ ID NO: 80561.

The probes having the nucleotide sequence represented by SEQ ID NOS: 141255, 141256, 141257, 141258, 141259, 141260, 141261, 141262, 141263, 141264, 141265 were selected from the ORFs having the nucleotide sequence represented by SEQ ID NO: 80562

The probes having the nucleotide sequence represented by SEQ ID NOS: 141266, 141267, 141268, 141269, 141270, 141271, 141272, 141273, 141274, 141275, 141276 were selected from the ORFs having the nucleotide sequence represented by SEQ ID NO: 80563

The probes having the nucleotide sequence represented by SEQ ID NOS: 141277, 141278, 141279, 141280, 141281, 141282, 141283, 141284, 141285, 141286, 141287 were selected from the ORFs having the nucleotide sequence represented by SEQ ID NO: 80564

The probes having the nucleotide sequence represented by SEQ ID NOS: 141288, 141289, 141290, 141291, 141292, 141293, 141294, 141295, 141296, 141297, 141298 were selected from the ORFs having the nucleotide sequence represented by SEQ ID NO: 80565

The probes having the nucleotide sequence represented by SEQ ID NOS: 141299, 141300, 141301, 141302, 141303, 141304, 141305, 141306, 141307, 141308, 141309 were selected from the ORFs having the nucleotide sequence represented by SEQ ID NO: 80566

The probes having the nucleotide sequence represented by SEQ ID NOS: 141310, 141311, 141312, 141313, 141314, 141315, 141316, 141317, 141318, 141319, 141320 were selected from the ORFs having the nucleotide sequence represented by SEQ ID NO: 80567

The probes having the nucleotide sequence represented by SEQ ID NOS: 141321, 141322, 141323, 141324, 141325, 141326, 141327, 141328, 141329, 141330, 141331 were selected from the ORFs having the nucleotide sequence represented by SEQ ID NO: 80568

The probes having the nucleotide sequence represented by SEQ ID NOS: 141332, 141333, 141334, 141335, 141336, 141337, 141338, 141339, 141340, 141341, 141342 were selected from the ORFs having the nucleotide sequence represented by SEQ ID NO: 80569

The probes having the nucleotide sequence represented by SEQ ID NOS: 141343, 141344, 141345, 141346, 141347, 141348, 141349, 141350, 141351, 141352, 141353 were selected from the ORFs having the nucleotide sequence represented by SEQ ID NO: 80570.

The probes having the nucleotide sequence represented by SEQ ID NOS: 141354, 141355, 141356, 141357, 141358, 141359, 141360, 141361, 141362, 141363, 141364 were selected from the ORFs having the nucleotide sequence represented by SEQ ID NO: 80571

The probes having the nucleotide sequence represented by SEQ ID NOS: 141365, 141366, 141367, 141368, 141369, 141370, 141371, 141372, 141373, 141374, 141375 were selected from the ORFs having the nucleotide sequence represented by SEQ ID NO: 80572.

The probes having the nucleotide sequence represented by SEQ ID NOS: 141376, 141377, 141378, 141379, 141380, 141381, 141382, 141383, 141384, 141385, 141386 were selected from the ORFs having the nucleotide sequence represented by SEQ ID NO: 80573

The probes having the nucleotide sequence represented by SEQ ID NOS: 141387, 141388, 141389, 141390, 141391, 141392, 141393, 141394, 141395, 141396, 141397 were selected from the ORFs having the nucleotide sequence represented by SEQ ID NO: 80574

The probes having the nucleotide sequence represented by SEQ ID NOS: 141398, 141399, 141400, 141401, 141402, 141403, 141404, 141405, 141406, 141407, 141408 were selected from the ORFs having the nucleotide sequence represented by SEQ ID NO: 80575

The probes having the nucleotide sequence represented by SEQ ID NOS: 141409, 141410, 141411, 141412, 141413, 141414, 141415, 141416, 141417, 141418, 141419 were selected from the ORFs having the nucleotide sequence represented by SEQ ID NO: 80576

The probes having the nucleotide sequence represented by SEQ ID NOS: 141420, 141421, 141422, 141423, 141424, 141425, 141426, 141427, 141428, 141429, 141430 were selected from the ORFs having the nucleotide sequence represented by SEQ ID NO: 80577

The probes having the nucleotide sequence represented by SEQ ID NOS: 141431, 141432, 141433, 141434, 141435, 141436, 141437, 141438, 141439, 141440, 141441 were selected from the ORFs having the nucleotide sequence represented by SEQ ID NO: 80578

The probes having the nucleotide sequence represented by SEQ ID NOS: 141442, 141443, 141444, 141445, 141446, 141447, 141448, 141449, 141450, 141451, 141452 were selected from the ORFs having the nucleotide sequence represented by SEQ ID NO: 80579

The probes having the nucleotide sequence represented by SEQ ID NOS: 141453, 141454, 141455, 141456, 141457, 141458, 141459, 141460, 141461, 141462, 141463 were selected from the ORFs having the nucleotide sequence represented by SEQ ID NO: 80580

The probes having the nucleotide sequence represented by SEQ ID NOS: 141464, 141465, 141466, 141467, 141468, 141469, 141470, 141471, 141472, 141473, 141474 were selected from the ORFs having the nucleotide sequence represented by SEQ ID NO: 80581

The probes having the nucleotide sequence represented by SEQ ID NOS: 141475, 141476, 141477, 141478, 141479, 141480, 141481, 141482, 141483, 141484, 141485 were selected from the ORFs having the nucleotide sequence represented by SEQ ID NO: 80582

The probes having the nucleotide sequence represented by SEQ ID NOS: 141486, 141487, 141488, 141489, 141490, 141491, 141492, 141493, 141494, 141495, 141496 were selected from the ORFs having the nucleotide sequence represented by SEQ ID NO: 80583.

The probes having the nucleotide sequence represented by SEQ ID NOS: 141497, 141498, 141499, 141500, 141501, 141502, 141503, 141504, 141505, 141506, 141507 were selected from the ORFs having the nucleotide sequence represented by SEQ ID NO: 80584

The probes having the nucleotide sequence represented by SEQ ID NOS: 141508, 141509, 141510, 141511, 141512, 141513, 141514, 141515, 141516, 141517, 141518 were selected from the ORFs having the nucleotide sequence represented by SEQ ID NO: 80585

The probes having the nucleotide sequence represented by SEQ ID NOS: 141519, 141520, 141521, 141522, 141523, 141524, 141525, 141526, 141527, 141528, 141529 were selected from the ORFs having the nucleotide sequence represented by SEQ ID NO: 80586

The probes having the nucleotide sequence represented by SEQ ID NOS: 141530, 141531, 141532, 141533, 141534, 141535, 141536, 141537, 141538, 141539, 141540 were selected from the ORFs having the nucleotide sequence represented by SEQ ID NO: 80587

The probes having the nucleotide sequence represented by SEQ ID NOS: 141541, 141542, 141543, 141544, 141545, 141546, 141547, 141548, 141549, 141550, 141551 were selected from the ORFs having the nucleotide sequence represented by SEQ ID NO: 80588

The probes having the nucleotide sequence represented by SEQ ID NOS: 141552, 141553, 141554, 141555, 141556, 141557, 141558, 141559, 141560, 141561, 141562 were selected from the ORFs having the nucleotide sequence represented by SEQ ID NO: 80589

The probes having the nucleotide sequence represented by SEQ ID NOS: 141563, 141564, 141565, 141566, 141567, 141568, 141569, 141570, 141571, 141572, 141573 were selected from the ORFs having the nucleotide sequence represented by SEQ ID NO: 80590

The probes having the nucleotide sequence represented by SEQ ID NOS: 141574, 141575, 141576, 141577, 141578, 141579, 141580, 141581, 141582, 141583, 141584 were selected from the ORFs having the nucleotide sequence represented by SEQ ID NO: 80591

The probes having the nucleotide sequence represented by SEQ ID NOS: 141585, 141586, 141587, 141588, 141589, 141590, 141591, 141592, 141593, 141594, 141595 were selected from the ORFs having the nucleotide sequence represented by SEQ ID NO: 80592

The probes having the nucleotide sequence represented by SEQ ID NOS: 141596, 141597, 141598, 141599, 141600, 141601, 141602, 141603, 141604, 141605, 141606 were selected from the ORFs having the nucleotide sequence represented by SEQ ID NO: 80593

The probes having the nucleotide sequence represented by SEQ ID NOS: 141607, 141608, 141609, 141610, 141611, 141612, 141613, 141614, 141615, 141616, 141617 were selected from the ORFs having the nucleotide sequence represented by SEQ ID NO: 80594.

The probes having the nucleotide sequence represented by SEQ ID NOS: 141618, 141619, 141620, 141621, 141622, 141623, 141624, 141625, 141626, 141627, 141628 were selected from the ORFs having the nucleotide sequence represented by SEQ ID NO: 80595

The probes having the nucleotide sequence represented by SEQ ID NOS: 141629, 141630, 141631, 141632, 141633, 141634, 141635, 141636, 141637, 141638; 141639 were selected from the ORFs having the nucleotide sequence represented by SEQ ID NO: 80596

The probes having the nucleotide sequence represented by SEQ ID NOS: 141640, 141641, 141642, 141643, 141644, 141645, 141646, 141647, 141648, 141649, 141650 were selected from the ORFs having the nucleotide sequence represented by SEQ ID NO: 80597

The probes having the nucleotide sequence represented by SEQ ID NOS: 141651, 141652, 141653, 141654, 141655, 141656, 141657, 141658, 141659, 141660, 141661 were selected from the ORFs having the nucleotide sequence represented by SEQ ID NO: 80598

The probes having the nucleotide sequence represented by SEQ ID NOS: 141662, 141663, 141664, 141665, 141666, 141667, 141668, 141669, 141670, 141671, 141672 were selected from the ORFs having the nucleotide sequence represented by SEQ ID NO: 80599

The probes having the nucleotide sequence represented by SEQ ID NOS: 141673, 141674, 141675, 141676, 141677, 141678, 141679, 141680, 141681, 141682, 141683 were selected from the ORFs having the nucleotide sequence represented by SEQ ID NO: 80600

The probes having the nucleotide sequence represented by SEQ ID NOS: 141684, 141685, 141686, 141687, 141688, 141689, 141690, 141691, 141692, 141693, 141694 were selected from the ORFs having the nucleotide sequence represented by SEQ ID NO: 80601

The probes having the nucleotide sequence represented by SEQ ID NOS: 141695, 141696, 141697, 141698, 141699, 141700, 141701, 141702, 141703, 141704, 141705 were selected from the ORFs having the nucleotide sequence represented by SEQ ID NO: 80602

The probes having the nucleotide sequence represented by SEQ ID NOS: 141706, 141707, 141708, 141709, 141710, 141711, 141712, 141713, 141714, 141715, 141716 were selected from the ORFs having the nucleotide sequence represented by SEQ ID NO: 80603

The probes having the nucleotide sequence represented by SEQ ID NOS: 141717, 141718, 141719, 141720, 141721, 141722, 141723, 141724, 141725, 141726, 141727 were selected from the ORFs having the nucleotide sequence represented by SEQ ID NO: 80604

The probes having the nucleotide sequence represented by SEQ ID NOS: 141728, 141729, 141730, 141731, 141732, 141733, 141734, 141735, 141736, 141737, 141738 were selected from the ORFs having the nucleotide sequence represented by SEQ ID NO: 80605.

The probes having the nucleotide sequence represented by SEQ ID NOS: 141739, 141740, 141741, 141742, 141743, 141744, 141745, 141746, 141747, 141748, 141749 were selected from the ORFs having the nucleotide sequence represented by SEQ ID NO: 80606

The probes having the nucleotide sequence represented by SEQ ID NOS: 141750, 141751, 141752, 141753, 141754, 141755, 141756, 141757, 141758, 141759, 141760 were selected from the ORFs having the nucleotide sequence represented by SEQ ID NO: 80607

The probes having the nucleotide sequence represented by SEQ ID NOS: 141761, 141762, 141763, 141764, 141765, 141766, 141767, 141768, 141769, 141770, 141771 were selected from the ORFs having the nucleotide sequence represented by SEQ ID NO: 80608

The probes having the nucleotide sequence represented by SEQ ID NOS: 141772, 141773, 141774, 141775, 141776, 141777, 141778, 141779, 141780, 141781, 141782 were selected from the ORFs having the nucleotide sequence represented by SEQ ID NO: 80609

The probes having the nucleotide sequence represented by SEQ ID NOS: 141783, 141784, 141785, 141786, 141787, 141788, 141789, 141790, 141791, 141792, 141793 were selected from the ORFs having the nucleotide sequence represented by SEQ ID NO: 80610

The probes having the nucleotide sequence represented by SEQ ID NOS: 141794, 141795, 141796, 141797, 141798, 141799, 141800, 141801, 141802, 141803, 141804 were selected from the ORFs having the nucleotide sequence represented by SEQ ID NO: 80611

The probes having the nucleotide sequence represented by SEQ ID NOS: 141805, 141806, 141807, 141808, 141809, 141810, 141811, 141812, 141813, 141814, 141815 were selected from the ORFs having the nucleotide sequence represented by SEQ ID NO: 80612

The probes having the nucleotide sequence represented by SEQ ID NOS: 141816, 141817, 141818, 141819, 141820, 141821, 141822, 141823, 141824, 141825, 141826 were selected from the ORFs having the nucleotide sequence represented by SEQ ID NO: 80613

The probes having the nucleotide sequence represented by SEQ ID NOS: 141827, 141828, 141829, 141830, 141831, 141832, 141833, 141834, 141835, 141836, 141837 were selected from the ORFs having the nucleotide sequence represented by SEQ ID NO: 80614

The probes having the nucleotide sequence represented by SEQ ID NOS: 141838, 141839, 141840, 141841, 141842, 141843, 141844, 141845, 141846, 141847, 141848 were selected from the ORFs having the nucleotide sequence represented by SEQ ID NO: 80615

The probes having the nucleotide sequence represented by SEQ ID NOS: 141849, 141850, 141851, 141852, 141853, 141854, 141855, 141856, 141857, 141858, 141859, 141860, 141861, 141862, 141863, 141864, 141865, 141866, 141867, 141868, 141869, 141870 were selected from the ORFs having the nucleotide sequence represented by SEQ ID NO: 80616.

The probes having the nucleotide sequence represented by SEQ ID NOS: 141871, 141872, 141873, 141874, 141875, 141876, 141877, 141878, 141879, 141880, 141881 were selected from the ORFs having the nucleotide sequence represented by SEQ ID NO: 80617

The probes having the nucleotide sequence represented by SEQ ID NOS: 141882, 141883, 141884, 141885, 141886, 141887, 141888, 141889, 141890, 141891, 141892 were selected from the ORFs having the nucleotide sequence represented by SEQ ID NO: 80618

The probes having the nucleotide sequence represented by SEQ ID NOS: 141893, 141894, 141895, 141896, 141897, 141898, 141899, 141900, 141901, 141902, 141903 were selected from the ORFs having the nucleotide sequence represented by SEQ ID NO: 80619

The probes having the nucleotide sequence represented by SEQ ID NOS: 141904, 141905, 141906, 141907, 141908, 141909, 141910, 141911, 141912, 141913, 141914 were selected from the ORFs having the nucleotide sequence represented by SEQ ID NO: 80620

The probes having the nucleotide sequence represented by SEQ ID NOS: 141915, 141916, 141917, 141918, 141919, 141920, 141921, 141922, 141923, 141924, 141925 were selected from the ORFs having the nucleotide sequence represented by SEQ ID NO: 80621

The probes having the nucleotide sequence represented by SEQ ID NOS: 141926, 141927, 141928, 141929, 141930, 141931, 141932, 141933, 141934, 141935, 141936 were selected from the ORFs having the nucleotide sequence represented by SEQ ID NO: 80622

The probes having the nucleotide sequence represented by SEQ ID NOS: 141937, 141938, 141939, 141940, 141941, 141942, 141943, 141944, 141945, 141946, 141947 were selected from the ORFs having the nucleotide sequence represented by SEQ ID NO: 80623

The probes having the nucleotide sequence represented by SEQ ID NOS: 141948, 141949, 141950, 141951, 141952, 141953, 141954, 141955, 141956, 141957, 141958 were selected from the ORFs having the nucleotide sequence represented by SEQ ID NO: 80624

The probes having the nucleotide sequence represented by SEQ ID NOS: 141959, 141960, 141961, 141962, 141963, 141964, 141965, 141966, 141967, 141968, 141969 were selected from the ORFs having the nucleotide sequence represented by SEQ ID NO: 80625

The probes having the nucleotide sequence represented by SEQ ID NOS: 141970, 141971, 141972, 141973, 141974, 141975, 141976, 141977, 141978, 141979, 141980 were selected from the ORFs having the nucleotide sequence represented by SEQ ID NO: 80626

The probes having the nucleotide sequence represented by SEQ ID NOS: 141981, 141982, 141983, 141984, 141985, 141986, 141987, 141988, 141989, 141990, 141991 were selected from the ORFs having the nucleotide sequence represented by SEQ ID NO: 80627.

The probes having the nucleotide sequence represented by SEQ ID NOS: 141992, 141993, 141994, 141995, 141996, 141997, 141998, 141999, 142000, 142001, 142002 were selected from the ORFs having the nucleotide sequence represented by SEQ ID NO: 80628

The probes having the nucleotide sequence represented by SEQ ID NOS: 142003, 142004, 142005, 142006, 142007, 142008, 142009, 142010, 142011, 142012, 142013 were selected from the ORFs having the nucleotide sequence represented by SEQ ID NO: 80629

The probes having the nucleotide sequence represented by SEQ ID NOS: 142014, 142015, 142016, 142017, 142018, 142019, 142020, 142021, 142022, 142023, 142024 were selected from the ORFs having the nucleotide sequence represented by SEQ ID NO: 80630

The probes having the nucleotide sequence represented by SEQ ID NOS: 142025, 142026, 142027, 142028, 142029, 142030, 142031, 142032, 142033, 142034, 142035 were selected from the ORFs having the nucleotide sequence represented by SEQ ID NO: 80631

The probes having the nucleotide sequence represented by SEQ ID NOS: 142036, 142037, 142038, 142039, 142040, 142041, 142042, 142043, 142044, 142045, 142046 were selected from the ORFs having the nucleotide sequence represented by SEQ ID NO: 80632

The probes having the nucleotide sequence represented by SEQ ID NOS: 142047, 142048, 142049, 142050, 142051, 142052, 142053, 142054, 142055, 142056, 142057 were selected from the ORFs having the nucleotide sequence represented by SEQ ID NO: 80633

The probes having the nucleotide sequence represented by SEQ ID NOS: 142058, 142059, 142060, 142061, 142062, 142063, 142064, 142065, 142066, 142067, 142068 were selected from the ORFs having the nucleotide sequence represented by SEQ ID NO: 80634

The probes having the nucleotide sequence represented by SEQ ID NOS: 142069, 142070, 142071, 142072, 142073, 142074, 142075, 142076, 142077, 142078, 142079 were selected from the ORFs having the nucleotide sequence represented by SEQ ID NO: 80635

The probes having the nucleotide sequence represented by SEQ ID NOS: 142080, 142081, 142082, 142083, 142084, 142085, 142086, 142087, 142088, 142089, 142090 were selected from the ORFs having the nucleotide sequence represented by SEQ ID NO: 80636

The probes having the nucleotide sequence represented by SEQ ID NOS: 142091, 142092, 142093, 142094, 142095, 142096, 142097, 142098, 142099, 142100, 142101 were selected from the ORFs having the nucleotide sequence represented by SEQ ID NO: 80637

The probes having the nucleotide sequence represented by SEQ ID NOS: 142102, 142103, 142104, 142105, 142106, 142107, 142108, 142109, 142110, 142111, 142112 were selected from the ORFs having the nucleotide sequence represented by SEQ ID NO: 80638.

The probes having the nucleotide sequence represented by SEQ ID NOS: 142113, 142114, 142115, 142116, 142117, 142118, 142119, 142120, 142121, 142122, 142123 were selected from the ORFs having the nucleotide sequence represented by SEQ ID NO: 80639

The probes having the nucleotide sequence represented by SEQ ID NOS: 142124, 142125, 142126, 142127, 142128, 142129, 142130, 142131, 142132, 142133, 142134 were selected from the ORFs having the nucleotide sequence represented by SEQ ID NO: 80640

The probes having the nucleotide sequence represented by SEQ ID NOS: 142135, 142136, 142137, 142138, 142139, 142140, 142141, 142142, 142143, 142144, 142145 were selected from the ORFs having the nucleotide sequence represented by SEQ ID NO: 80641

The probes having the nucleotide sequence represented by SEQ ID NOS: 142146, 142147, 142148, 142149, 142150, 142151, 142152, 142153, 142154, 142155, 142156 were selected from the ORFs having the nucleotide sequence represented by SEQ ID NO: 80642

The probes having the nucleotide sequence represented by SEQ ID NOS: 142157, 142158, 142159, 142160, 142161, 142162, 142163, 142164, 142165, 142166, 142167 were selected from the ORFs having the nucleotide sequence represented by SEQ ID NO: 80643

The probes having the nucleotide sequence represented by SEQ ID NOS: 142168, 142169, 142170, 142171, 142172, 142173, 142174, 142175, 142176, 142177, 142178 were selected from the ORFs having the nucleotide sequence represented by SEQ ID NO: 80644

The probes having the nucleotide sequence represented by SEQ ID NOS: 142179, 142180, 142181, 142182, 142183, 142184, 142185, 142186, 142187, 142188, 142189 were selected from the ORFs having the nucleotide sequence represented by SEQ ID NO: 80645

The probes having the nucleotide sequence represented by SEQ ID NOS: 142190, 142191, 142192, 142193, 142194, 142195, 142196, 142197, 142198, 142199, 142200 were selected from the ORFs having the nucleotide sequence represented by SEQ ID NO: 80646

The probes having the nucleotide sequence represented by SEQ ID NOS: 142201, 142202, 142203, 142204, 142205, 142206, 142207, 142208, 142209, 142210, 142211 were selected from the ORFs having the nucleotide sequence represented by SEQ ID NO: 80647

The probes having the nucleotide sequence represented by SEQ ID NOS: 142212, 142213, 142214, 142215, 142216, 142217, 142218, 142219, 142220, 142221, 142222 were selected from the ORFs having the nucleotide sequence represented by SEQ ID NO: 80648

The probes having the nucleotide sequence represented by SEQ ID NOS: 142223, 142224, 142225, 142226, 142227, 142228, 142229, 142230, 142231, 142232, 142233 were selected from the ORFs having the nucleotide sequence represented by SEQ ID NO: 80649.

The probes having the nucleotide sequence represented by SEQ ID NOS: 142234, 142235, 142236, 142237, 142238, 142239, 142240, 142241, 142242, 142243, 142244 were selected from the ORFs having the nucleotide sequence represented by SEQ ID NO: 80650

The probes having the nucleotide sequence represented by SEQ ID NOS: 142245, 142246, 142247, 142248, 142249, 142250, 142251, 142252, 142253, 142254, 142255 were selected from the ORFs having the nucleotide sequence represented by SEQ ID NO: 80651

The probes having the nucleotide sequence represented by SEQ ID NOS: 142256, 142257, 142258, 142259, 142260, 142261, 142262, 142263, 142264, 142265, 142266 were selected from the ORFs having the nucleotide sequence represented by SEQ ID NO: 80652

The probes having the nucleotide sequence represented by SEQ ID NOS: 142267, 142268, 142269, 142270, 142271, 142272, 142273, 142274, 142275, 142276, 142277 were selected from the ORFs having the nucleotide sequence represented by SEQ ID NO: 80653

The probes having the nucleotide sequence represented by SEQ ID NOS: 142278, 142279, 142280, 142281, 142282, 142283, 142284, 142285, 142286, 142287, 142288 were selected from the ORFs having the nucleotide sequence represented by SEQ ID NO: 80654

The probes having the nucleotide sequence represented by SEQ ID NOS: 142289, 142290, 142291, 142292, 142293, 142294, 142295, 142296, 142297, 142298, 142299 were selected from the ORFs having the nucleotide sequence represented by SEQ ID NO: 80655

The probes having the nucleotide sequence represented by SEQ ID NOS: 142300, 142301, 142302, 142303, 142304, 142305, 142306, 142307, 142308, 142309, 142310 were selected from the ORFs having the nucleotide sequence represented by SEQ ID NO: 80656

The probes having the nucleotide sequence represented by SEQ ID NOS: 142311, 142312, 142313, 142314, 142315, 142316, 142317, 142318, 142319, 142320, 142321 were selected from the ORFs having the nucleotide sequence represented by SEQ ID NO: 80657

The probes having the nucleotide sequence represented by SEQ ID NOS: 142322, 142323, 142324, 142325, 142326, 142327, 142328, 142329, 142330, 142331, 142332, 142333, 142334, 142335, 142336, 142337, 142338, 142339, 142340, 142341, 142342, 142343, 142344, 142345, 142346, 142347, 142348, 142349, 142350, 142351, 142352, 142353, 142354 were selected from the ORFs having the nucleotide sequence represented by SEQ ID NO: 80658

The probes having the nucleotide sequence represented by SEQ ID NOS: 142355, 142356, 142357, 142358, 142359, 142360, 142361, 142362, 142363, 142364, 142365 were selected from the ORFs having the nucleotide sequence represented by SEQ ID NO: 80659

The probes having the nucleotide sequence represented by SEQ ID NOS: 142366, 142367, 142368, 142369, 142370, 142371, 142372, 142373, 142374, 142375, 142376 were selected from the ORFs having the nucleotide sequence represented by SEQ ID NO: 80660

The probes having the nucleotide sequence represented by SEQ ID NOS: 142377, 142378, 142379, 142380, 142381, 142382, 142383, 142384, 142385, 142386, 142387 were selected from the ORFs having the nucleotide sequence represented by SEQ ID NO: 80661

The probes having the nucleotide sequence represented by SEQ ID NOS: 142388, 142389, 142390, 142391, 142392, 142393, 142394, 142395, 142396, 142397, 142398 were selected from the ORFs having the nucleotide sequence represented by SEQ ID NO: 80662

The probes having the nucleotide sequence represented by SEQ ID NOS: 142399, 142400, 142401, 142402, 142403, 142404, 142405, 142406, 142407, 142408, 142409 were selected from the ORFs having the nucleotide sequence represented by SEQ ID NO: 80663

The probes having the nucleotide sequence represented by SEQ ID NOS: 142410, 142411, 142412, 142413, 142414, 142415, 142416, 142417, 142418, 142419, 142420 were selected from the ORFs having the nucleotide sequence represented by SEQ ID NO: 80664

The probes having the nucleotide sequence represented by SEQ ID NOS: 142421, 142422, 142423, 142424, 142425, 142426, 142427, 142428, 142429, 142430, 142431 were selected from the ORFs having the nucleotide sequence represented by SEQ ID NO: 80665.

The probes having the nucleotide sequence represented by SEQ ID NOS: 142432, 142433, 142434, 142435, 142436, 142437, 142438, 142439, 142440, 142441, 142442 were selected from the ORFs having the nucleotide sequence represented by SEQ ID NO: 80666

The probes having the nucleotide sequence represented by SEQ ID NOS: 142443, 142444, 142445, 142446, 142447, 142448, 142449, 142450, 142451, 142452, 142453 were selected from the ORFs having the nucleotide sequence represented by SEQ ID NO: 80667

The probes having the nucleotide sequence represented by SEQ ID NOS: 142454, 142455, 142456, 142457, 142458, 142459, 142460, 142461, 142462, 142463, 142464 were selected from the ORFs having the nucleotide sequence represented by SEQ ID NO: 80668

The probes having the nucleotide sequence represented by SEQ ID NOS: 142465, 142466, 142467, 142468, 142469, 142470, 142471, 142472, 142473, 142474, 142475 were selected from the ORFs having the nucleotide sequence represented by SEQ ID NO: 80669

The probes having the nucleotide sequence represented by SEQ ID NOS: 142476, 142477, 142478, 142479, 142480, 142481, 142482, 142483, 142484, 142485, 142486 were selected from the ORFs having the nucleotide sequence represented by SEQ ID NO: 80670

The probes having the nucleotide sequence represented by SEQ ID NOS: 142487, 142488, 142489, 142490, 142491, 142492, 142493, 142494, 142495, 142496, 142497 were selected from the ORFs having the nucleotide sequence represented by SEQ ID NO: 80671

The probes having the nucleotide sequence represented by SEQ ID NOS: 142498, 142499, 142500, 142501, 142502, 142503, 142504, 142505, 142506, 142507, 142508 were selected from the ORFs having the nucleotide sequence represented by SEQ ID NO: 80672

The probes having the nucleotide sequence represented by SEQ ID NOS: 142509, 142510, 142511, 142512, 142513, 142514, 142515, 142516, 142517, 142518, 142519 were selected from the ORFs having the nucleotide sequence represented by SEQ ID NO: 80673

The probes having the nucleotide sequence represented by SEQ ID NOS: 142520, 142521, 142522, 142523, 142524, 142525, 142526, 142527, 142528, 142529, 142530 were selected from the ORFs having the nucleotide sequence represented by SEQ ID NO: 80674

The probes having the nucleotide sequence represented by SEQ ID NOS: 142531, 142532, 142533, 142534, 142535, 142536, 142537, 142538, 142539, 142540, 142541 were selected from the ORFs having the nucleotide sequence represented by SEQ ID NO: 80675

The probes having the nucleotide sequence represented by SEQ ID NOS: 142542, 142543, 142544, 142545, 142546, 142547, 142548, 142549, 142550, 142551, 142552 were selected from the ORFs having the nucleotide sequence represented by SEQ ID NO: 80676.

The probes having the nucleotide sequence represented by SEQ ID NOS: 142553, 142554, 142555, 142556, 142557, 142558, 142559, 142560, 142561, 142562, 142563 were selected from the ORFs having the nucleotide sequence represented by SEQ ID NO: 80677

The probes having the nucleotide sequence represented by SEQ ID NOS: 142564, 142565, 142566, 142567, 142568, 142569, 142570, 142571, 142572, 142573, 142574 were selected from the ORFs having the nucleotide sequence represented by SEQ ID NO: 80678

The probes having the nucleotide sequence represented by SEQ ID NOS: 142575, 142576, 142577, 142578, 142579, 142580, 142581, 142582, 142583, 142584, 142585 were selected from the ORFs having the nucleotide sequence represented by SEQ ID NO: 80679

The probes having the nucleotide sequence represented by SEQ ID NOS: 142586, 142587, 142588, 142589, 142590, 142591, 142592, 142593, 142594, 142595, 142596 were selected from the ORFs having the nucleotide sequence represented by SEQ ID NO: 80680

The probes having the nucleotide sequence represented by SEQ ID NOS: 142597, 142598, 142599, 142600, 142601, 142602, 142603, 142604, 142605, 142606, 142607 were selected from the ORFs having the nucleotide sequence represented by SEQ ID NO: 80681

The probes having the nucleotide sequence represented by SEQ ID NOS: 142608, 142609, 142610, 142611, 142612, 142613, 142614, 142615, 142616, 142617, 142618 were selected from the ORFs having the nucleotide sequence represented by SEQ ID NO: 80682

The probes having the nucleotide sequence represented by SEQ ID NOS: 142619, 142620, 142621, 142622, 142623, 142624, 142625, 142626, 142627, 142628, 142629 were selected from the ORFs having the nucleotide sequence represented by SEQ ID NO: 80683

The probes having the nucleotide sequence represented by SEQ ID NOS: 142630, 142631, 142632, 142633, 142634, 142635, 142636, 142637, 142638, 142639, 142640 were selected from the ORFs having the nucleotide sequence represented by SEQ ID NO: 80684

The probes having the nucleotide sequence represented by SEQ ID NOS: 142641, 142642, 142643, 142644, 142645, 142646, 142647, 142648, 142649, 142650, 142651 were selected from the ORFs having the nucleotide sequence represented by SEQ ID NO: 80685

The probes having the nucleotide sequence represented by SEQ ID NOS: 142652, 142653, 142654, 142655, 142656, 142657, 142658, 142659, 142660, 142661, 142662 were selected from the ORFs having the nucleotide sequence represented by SEQ ID NO: 80686

The probes having the nucleotide sequence represented by SEQ ID NOS: 142663, 142664, 142665, 142666, 142667, 142668, 142669, 142670, 142671, 142672, 142673 were selected from the ORFs having the nucleotide sequence represented by SEQ ID NO: 80687.

The probes having the nucleotide sequence represented by SEQ ID NOS: 142674, 142675, 142676, 142677, 142678, 142679, 142680, 142681, 142682, 142683, 142684 were selected from the ORFs having the nucleotide sequence represented by SEQ ID NO: 80688

The probes having the nucleotide sequence represented by SEQ ID NOS: 142685, 142686, 142687, 142688, 142689, 142690, 142691, 142692, 142693, 142694, 142695 were selected from the ORFs having the nucleotide sequence represented by SEQ ID NO: 80689

The probes having the nucleotide sequence represented by SEQ ID NOS: 142696, 142697, 142698, 142699, 142700, 142701, 142702, 142703, 142704, 142705, 142706 were selected from the ORFs having the nucleotide sequence represented by SEQ ID NO: 80690

The probes having the nucleotide sequence represented by SEQ ID NOS: 142707, 142708, 142709, 142710, 142711, 142712, 142713, 142714, 142715, 142716, 142717 were selected from the ORFs having the nucleotide sequence represented by SEQ ID NO: 80691

The probes having the nucleotide sequence represented by SEQ ID NOS: 142718, 142719, 142720, 142721, 142722, 142723, 142724, 142725, 142726, 142727, 142728 were selected from the ORFs having the nucleotide sequence represented by SEQ ID NO: 80692

The probes having the nucleotide sequence represented by SEQ ID NOS: 142729, 142730, 142731, 142732, 142733, 142734, 142735, 142736, 142737, 142738, 142739 were selected from the ORFs having the nucleotide sequence represented by SEQ ID NO: 80693

The probes having the nucleotide sequence represented by SEQ ID NOS: 142740, 142741, 142742, 142743, 142744, 142745, 142746, 142747, 142748, 142749, 142750 were selected from the ORFs having the nucleotide sequence represented by SEQ ID NO: 80694

The probes having the nucleotide sequence represented by SEQ ID NOS: 142751, 142752, 142753, 142754, 142755, 142756, 142757, 142758, 142759, 142760, 142761 were selected from the ORFs having the nucleotide sequence represented by SEQ ID NO: 80695

The probes having the nucleotide sequence represented by SEQ ID NOS: 142762, 142763, 142764, 142765, 142766, 142767, 142768, 142769, 142770, 142771, 142772 were selected from the ORFs having the nucleotide sequence represented by SEQ ID NO: 80696

The probes having the nucleotide sequence represented by SEQ ID NOS: 142773, 142774, 142775, 142776, 142777, 142778, 142779, 142780, 142781, 142782, 142783 were selected from the ORFs having the nucleotide sequence represented by SEQ ID NO: 80697

The probes having the nucleotide sequence represented by SEQ ID NOS: 142784, 142785, 142786, 142787, 142788, 142789, 142790, 142791, 142792, 142793, 142794 were selected from the ORFs having the nucleotide sequence represented by SEQ ID NO: 80698.

The probes having the nucleotide sequence represented by SEQ ID NOS: 142795, 142796, 142797, 142798, 142799, 142800, 142801, 142802, 142803, 142804, 142805 were selected from the ORFs having the nucleotide sequence represented by SEQ ID NO: 80699

The probes having the nucleotide sequence represented by SEQ ID NOS: 142806, 142807, 142808, 142809, 142810, 142811, 142812, 142813, 142814, 142815, 142816 were selected from the ORFs having the nucleotide sequence represented by SEQ ID NO: 80700

The probes having the nucleotide sequence represented by SEQ ID NOS: 142817, 142818, 142819, 142820, 142821, 142822, 142823, 142824, 142825, 142826, 142827 were selected from the ORFs having the nucleotide sequence represented by SEQ ID NO: 80701

The probes having the nucleotide sequence represented by SEQ ID NOS: 142828, 142829, 142830, 142831, 142832, 142833, 142834, 142835, 142836, 142837, 142838 were selected from the ORFs having the nucleotide sequence represented by SEQ ID NO: 80702

The probes having the nucleotide sequence represented by SEQ ID NOS: 142839, 142840, 142841, 142842, 142843, 142844, 142845, 142846, 142847, 142848, 142849 were selected from the ORFs having the nucleotide sequence represented by SEQ ID NO: 80703

The probes having the nucleotide sequence represented by SEQ ID NOS: 142850, 142851, 142852, 142853, 142854, 142855, 142856, 142857, 142858, 142859, 142860 were selected from the ORFs having the nucleotide sequence represented by SEQ ID NO: 80704

The probes having the nucleotide sequence represented by SEQ ID NOS: 142861, 142862, 142863, 142864, 142865, 142866, 142867, 142868, 142869, 142870, 142871 were selected from the ORFs having the nucleotide sequence represented by SEQ ID NO: 80705

The probes having the nucleotide sequence represented by SEQ ID NOS: 142872, 142873, 142874, 142875, 142876, 142877, 142878, 142879, 142880, 142881, 142882 were selected from the ORFs having the nucleotide sequence represented by SEQ ID NO: 80706

The probes having the nucleotide sequence represented by SEQ ID NOS: 142883, 142884, 142885, 142886, 142887, 142888, 142889, 142890, 142891, 142892, 142893 were selected from the ORFs having the nucleotide sequence represented by SEQ ID NO: 80707

The probes having the nucleotide sequence represented by SEQ ID NOS: 142894, 142895, 142896, 142897, 142898, 142899, 142900, 142901, 142902, 142903, 142904 were selected from the ORFs having the nucleotide sequence represented by SEQ ID NO: 80708

The probes having the nucleotide sequence represented by SEQ ID NOS: 142905, 142906, 142907, 142908, 142909, 142910, 142911, 142912, 142913, 142914, 142915 were selected from the ORFs having the nucleotide sequence represented by SEQ ID NO: 80709.

The probes having the nucleotide sequence represented by SEQ ID NOS: 142916, 142917, 142918, 142919, 142920, 142921, 142922, 142923, 142924, 142925, 142926 were selected from the ORFs having the nucleotide sequence represented by SEQ ID NO: 80710

The probes having the nucleotide sequence represented by SEQ ID NOS: 142927, 142928, 142929, 142930, 142931, 142932, 142933, 142934, 142935, 142936, 142937 were selected from the ORFs having the nucleotide sequence represented by SEQ ID NO: 80711

The probes having the nucleotide sequence represented by SEQ ID NOS: 142938, 142939, 142940, 142941, 142942, 142943, 142944, 142945, 142946, 142947, 142948 were selected from the ORFs having the nucleotide sequence represented by SEQ ID NO: 80712

The probes having the nucleotide sequence represented by SEQ ID NOS: 142949, 142950, 142951, 142952, 142953, 142954, 142955, 142956, 142957, 142958, 142959 were selected from the ORFs having the nucleotide sequence represented by SEQ ID NO: 80713

The probes having the nucleotide sequence represented by SEQ ID NOS: 142960, 142961, 142962, 142963, 142964, 142965, 142966, 142967, 142968, 142969, 142970 were selected from the ORFs having the nucleotide sequence represented by SEQ ID NO: 80714

The probes having the nucleotide sequence represented by SEQ ID NOS: 142971, 142972, 142973, 142974, 142975, 142976, 142977, 142978, 142979, 142980, 142981 were selected from the ORFs having the nucleotide sequence represented by SEQ ID NO: 80715

The probes having the nucleotide sequence represented by SEQ ID NOS: 142982, 142983, 142984, 142985, 142986, 142987, 142988, 142989, 142990, 142991, 142992 were selected from the ORFs having the nucleotide sequence represented by SEQ ID NO: 80716

The probes having the nucleotide sequence represented by SEQ ID NOS: 142993, 142994, 142995, 142996, 142997, 142998, 142999, 143000, 143001, 143002, 143003 were selected from the ORFs having the nucleotide sequence represented by SEQ ID NO: 80717

The probes having the nucleotide sequence represented by SEQ ID NOS: 143004, 143005, 143006, 143007, 143008, 143009, 143010, 143011, 143012, 143013, 143014 were selected from the ORFs having the nucleotide sequence represented by SEQ ID NO: 80718

The probes having the nucleotide sequence represented by SEQ ID NOS: 143015, 143016, 143017, 143018, 143019, 143020, 143021, 143022, 143023, 143024, 143025 were selected from the ORFs having the nucleotide sequence represented by SEQ ID NO: 80719

The probes having the nucleotide sequence represented by SEQ ID NOS: 143026, 143027, 143028, 143029, 143030, 143031, 143032, 143033, 143034, 143035, 143036 were selected from the ORFs having the nucleotide sequence represented by SEQ ID NO: 80720.

The probes having the nucleotide sequence represented by SEQ ID NOS: 143037, 143038, 143039, 143040, 143041, 143042, 143043, 143044, 143045, 143046, 143047 were selected from the ORFs having the nucleotide sequence represented by SEQ ID NO: 80721

The probes having the nucleotide sequence represented by SEQ ID NOS: 143048, 143049, 143050, 143051, 143052, 143053, 143054, 143055, 143056, 143057, 143058 were selected from the ORFs having the nucleotide sequence represented by SEQ ID NO: 80722

The probes having the nucleotide sequence represented by SEQ ID NOS: 143059, 143060, 143061, 143062, 143063, 143064, 143065, 143066, 143067, 143068, 143069 were selected from the ORFs having the nucleotide sequence represented by SEQ ID NO: 80723

The probes having the nucleotide sequence represented by SEQ ID NOS: 143070, 143071, 143072, 143073, 143074, 143075, 143076, 143077, 143078, 143079, 143080 were selected from the ORFs having the nucleotide sequence represented by SEQ ID NO: 80724

The probes having the nucleotide sequence represented by SEQ ID NOS: 143081, 143082, 143083, 143084, 143085, 143086, 143087, 143088, 143089, 143090, 143091 were selected from the ORFs having the nucleotide sequence represented by SEQ ID NO: 80725

The probes having the nucleotide sequence represented by SEQ ID NOS: 143092, 143093, 143094, 143095, 143096, 143097, 143098, 143099, 143100, 143101, 143102 were selected from the ORFs having the nucleotide sequence represented by SEQ ID NO: 80726

The probes having the nucleotide sequence represented by SEQ ID NOS: 143103, 143104, 143105, 143106, 143107, 143108, 143109, 143110, 143111, 143112, 143113 were selected from the ORFs having the nucleotide sequence represented by SEQ ID NO: 80727

The probes having the nucleotide sequence represented by SEQ ID NOS: 143114, 143115, 143116, 143117, 143118, 143119, 143120, 143121, 143122, 143123, 143124 were selected from the ORFs having the nucleotide sequence represented by SEQ ID NO: 80728

The probes having the nucleotide sequence represented by SEQ ID NOS: 143125, 143126, 143127, 143128, 143129, 143130, 143131, 143132, 143133, 143134, 143135 were selected from the ORFs having the nucleotide sequence represented by SEQ ID NO: 80729

The probes having the nucleotide sequence represented by SEQ ID NOS: 143136, 143137, 143138, 143139, 143140, 143141, 143142, 143143, 143144, 143145, 143146 were selected from the ORFs having the nucleotide sequence represented by SEQ ID NO: 80730

The probes having the nucleotide sequence represented by SEQ ID NOS: 143147, 143148, 143149, 143150, 143151, 143152, 143153, 143154, 143155, 143156, 143157 were selected from the ORFs having the nucleotide sequence represented by SEQ ID NO: 80731.

The probes having the nucleotide sequence represented by SEQ ID NOS: 143158, 143159, 143160, 143161, 143162, 143163, 143164, 143165, 143166, 143167, 143168 were selected from the ORFs having the nucleotide sequence represented by SEQ ID NO: 80732

The probes having the nucleotide sequence represented by SEQ ID NOS: 143169, 143170, 143171, 143172, 143173, 143174, 143175, 143176, 143177, 143178, 143179 were selected from the ORFs having the nucleotide sequence represented by SEQ ID NO: 80733

The probes having the nucleotide sequence represented by SEQ ID NOS: 143180, 143181, 143182, 143183, 143184, 143185, 143186, 143187, 143188, 143189, 143190 were selected from the ORFs having the nucleotide sequence represented by SEQ ID NO: 80734

The probes having the nucleotide sequence represented by SEQ ID NOS: 143191, 143192, 143193, 143194, 143195, 143196, 143197, 143198, 143199, 143200, 143201 were selected from the ORFs having the nucleotide sequence represented by SEQ ID NO: 80735

The probes having the nucleotide sequence represented by SEQ ID NOS: 143202, 143203, 143204, 143205, 143206, 143207, 143208, 143209, 143210, 143211, 143212 were selected from the ORFs having the nucleotide sequence represented by SEQ ID NO: 80736

The probes having the nucleotide sequence represented by SEQ ID NOS: 143213, 143214, 143215, 143216, 143217, 143218, 143219, 143220, 143221, 143222, 143223 were selected from the ORFs having the nucleotide sequence represented by SEQ ID NO: 80737

The probes having the nucleotide sequence represented by SEQ ID NOS: 143224, 143225, 143226, 143227, 143228, 143229, 143230, 143231, 143232, 143233, 143234 were selected from the ORFs having the nucleotide sequence represented by SEQ ID NO: 80738

The probes having the nucleotide sequence represented by SEQ ID NOS: 143235, 143236, 143237, 143238, 143239, 143240, 143241, 143242; 143243, 143244, 143245 were selected from the ORFs having the nucleotide sequence represented by SEQ ID NO: 80739

The probes having the nucleotide sequence represented by SEQ ID NOS: 143246, 143247, 143248, 143249, 143250, 143251, 143252, 143253, 143254, 143255, 143256 were selected from the ORFs having the nucleotide sequence represented by SEQ ID NO: 80740

The probes having the nucleotide sequence represented by SEQ ID NOS: 143257, 143258, 143259, 143260, 143261, 143262, 143263, 143264, 143265, 143266, 143267 were selected from the ORFs having the nucleotide sequence represented by SEQ ID NO: 80741

The probes having the nucleotide sequence represented by SEQ ID NOS: 143268, 143269, 143270, 143271, 143272, 143273, 143274, 143275, 143276, 143277, 143278 were selected from the ORFs having the nucleotide sequence represented by SEQ ID NO: 80742.

The probes having the nucleotide sequence represented by SEQ ID NOS: 143279, 143280, 143281, 143282, 143283, 143284, 143285, 143286, 143287, 143288, 143289 were selected from the ORFs having the nucleotide sequence represented by SEQ ID NO: 80743

The probes having the nucleotide sequence represented by SEQ ID NOS: 143290, 143291, 143292, 143293, 143294, 143295, 143296, 143297, 143298, 143299, 143300 were selected from the ORFs having the nucleotide sequence represented by SEQ ID NO: 80744

The probes having the nucleotide sequence represented by SEQ ID NOS: 143301, 143302, 143303, 143304, 143305, 143306, 143307, 143308, 143309, 143310, 143311 were selected from the ORFs having the nucleotide sequence represented by SEQ ID NO: 80745

The probes having the nucleotide sequence represented by SEQ ID NOS: 143312, 143313, 143314, 143315, 143316, 143317, 143318, 143319, 143320, 143321, 143322 were selected from the ORFs having the nucleotide sequence represented by SEQ ID NO: 80746

The probes having the nucleotide sequence represented by SEQ ID NOS: 143323, 143324, 143325, 143326, 143327, 143328, 143329, 143330, 143331, 143332, 143333 were selected from the ORFs having the nucleotide sequence represented by SEQ ID NO: 80747

The probes having the nucleotide sequence represented by SEQ ID NOS: 143334, 143335, 143336, 143337, 143338, 143339, 143340, 143341, 143342, 143343, 143344 were selected from the ORFs having the nucleotide sequence represented by SEQ ID NO: 80748

The probes having the nucleotide sequence represented by SEQ ID NOS: 143345, 143346, 143347, 143348, 143349, 143350, 143351, 143352, 143353, 143354, 143355 were selected from the ORFs having the nucleotide sequence represented by SEQ ID NO: 80749

The probes having the nucleotide sequence represented by SEQ ID NOS: 143356, 143357, 143358, 143359, 143360, 143361, 143362, 143363, 143364, 143365, 143366 were selected from the ORFs having the nucleotide sequence represented by SEQ ID NO: 80750

The probes having the nucleotide sequence represented by SEQ ID NOS: 143367, 143368, 143369, 143370, 143371, 143372, 143373, 143374, 143375, 143376, 143377 were selected from the ORFs having the nucleotide sequence represented by SEQ ID NO: 80751

The probes having the nucleotide sequence represented by SEQ ID NOS: 143378, 143379, 143380, 143381, 143382, 143383, 143384, 143385, 143386, 143387, 143388 were selected from the ORFs having the nucleotide sequence represented by SEQ ID NO: 80752

The probes having the nucleotide sequence represented by SEQ ID NOS: 143389, 143390, 143391, 143392, 143393, 143394, 143395, 143396, 143397, 143398, 143399 were selected from the ORFs having the nucleotide sequence represented by SEQ ID NO: 80753.

The probes having the nucleotide sequence represented by SEQ ID NOS: 143400, 143401, 143402, 143403, 143404, 143405, 143406, 143407, 143408, 143409, 143410 were selected from the ORFs having the nucleotide sequence represented by SEQ ID NO: 80754

The probes having the nucleotide sequence represented by SEQ ID NOS: 143411, 143412, 143413, 143414, 143415, 143416, 143417, 143418, 143419, 143420, 143421, 143422, 143423, 143424, 143425, 143426, 143427, 143428, 143429, 143430, 143431, 143432 were selected from the ORFs having the nucleotide sequence represented by SEQ ID NO: 80755

The probes having the nucleotide sequence represented by SEQ ID NOS: 143433, 143434, 143435, 143436, 143437, 143438, 143439, 143440, 143441, 143442, 143443 were selected from the ORFs having the nucleotide sequence represented by SEQ ID NO: 80756

The probes having the nucleotide sequence represented by SEQ ID NOS: 143444, 143445, 143446, 143447, 143448, 143449, 143450, 143451, 143452, 143453, 143454 were selected from the ORFs having the nucleotide sequence represented by SEQ ID NO: 80757

The probes having the nucleotide sequence represented by SEQ ID NOS: 143455, 143456, 143457, 143458, 143459, 143460, 143461, 143462, 143463, 143464, 143465 were selected from the ORFs having the nucleotide sequence represented by SEQ ID NO: 80758

The probes having the nucleotide sequence represented by SEQ ID NOS: 143466, 143467, 143468, 143469, 143470, 143471, 143472, 143473, 143474, 143475, 143476 were selected from the ORFs having the nucleotide sequence represented by SEQ ID NO: 80759

The probes having the nucleotide sequence represented by SEQ ID NOS: 143477, 143478, 143479, 143480, 143481, 143482, 143483, 143484, 143485, 143486, 143487 were selected from the ORFs having the nucleotide sequence represented by SEQ ID NO: 80760

The probes having the nucleotide sequence represented by SEQ ID NOS: 143488, 143489, 143490, 143491, 143492, 143493, 143494, 143495, 143496, 143497, 143498 were selected from the ORFs having the nucleotide sequence represented by SEQ ID NO: 80761

The probes having the nucleotide sequence represented by SEQ ID NOS: 143499, 143500, 143501, 143502, 143503, 143504, 143505, 143506, 143507, 143508, 143509 were selected from the ORFs having the nucleotide sequence represented by SEQ ID NO: 80762

The probes having the nucleotide sequence represented by SEQ ID NOS: 143510, 143511, 143512, 143513, 143514, 143515, 143516, 143517, 143518, 143519, 143520 were selected from the ORFs having the nucleotide sequence represented by SEQ ID NO: 80763

The probes having the nucleotide sequence represented by SEQ ID NOS: 143521, 143522, 143523, 143524, 143525, 143526, 143527, 143528, 143529, 143530, 143531 were selected from the ORFs having the nucleotide sequence represented by SEQ ID NO: 80764.

The probes having the nucleotide sequence represented by SEQ ID NOS: 143532, 143533, 143534, 143535, 143536, 143537, 143538, 143539, 143540, 143541, 143542 were selected from the ORFs having the nucleotide sequence represented by SEQ ID NO: 80765

The probes having the nucleotide sequence represented by SEQ ID NOS: 143543, 143544, 143545, 143546, 143547, 143548, 143549, 143550, 143551, 143552, 143553 were selected from the ORFs having the nucleotide sequence represented by SEQ ID NO: 80766

The probes having the nucleotide sequence represented by SEQ ID NOS: 143554, 143555, 143556, 143557, 143558, 143559, 143560, 143561, 143562, 143563, 143564 were selected from the ORFs having the nucleotide sequence represented by SEQ ID NO: 80767

The probes having the nucleotide sequence represented by SEQ ID NOS: 143565, 143566, 143567, 143568, 143569, 143570, 143571, 143572, 143573, 143574, 143575 were selected from the ORFs having the nucleotide sequence represented by SEQ ID NO: 80768

The probes having the nucleotide sequence represented by SEQ ID NOS: 143576, 143577, 143578, 143579, 143580, 143581, 143582, 143583, 143584, 143585, 143586 were selected from the ORFs having the nucleotide sequence represented by SEQ ID NO: 80769

The probes having the nucleotide sequence represented by SEQ ID NOS: 143587, 143588, 143589, 143590, 143591, 143592, 143593, 143594, 143595, 143596, 143597 were selected from the ORFs having the nucleotide sequence represented by SEQ ID NO: 80770

The probes having the nucleotide sequence represented by SEQ ID NOS: 143598, 143599, 143600, 143601, 143602, 143603, 143604, 143605, 143606, 143607, 143608 were selected from the ORFs having the nucleotide sequence represented by SEQ ID NO: 80771

The probes having the nucleotide sequence represented by SEQ ID NOS: 143609, 143610, 143611, 143612, 143613, 143614, 143615, 143616, 143617, 143618, 143619 were selected from the ORFs having the nucleotide sequence represented by SEQ ID NO: 80772

The probes having the nucleotide sequence represented by SEQ ID NOS: 143620, 143621, 143622, 143623, 143624, 143625, 143626, 143627, 143628, 143629, 143630 were selected from the ORFs having the nucleotide sequence represented by SEQ ID NO: 80773

The probes having the nucleotide sequence represented by SEQ ID NOS: 143631, 143632, 143633, 143634, 143635, 143636, 143637, 143638, 143639, 143640, 143641 were selected from the ORFs having the nucleotide sequence represented by SEQ ID NO: 80774

The probes having the nucleotide sequence represented by SEQ ID NOS: 143642, 143643, 143644, 143645, 143646, 143647, 143648, 143649, 143650, 143651, 143652 were selected from the ORFs having the nucleotide sequence represented by SEQ ID NO: 80775.

The probes having the nucleotide sequence represented by SEQ ID NOS: 143653, 143654, 143655, 143656, 143657, 143658, 143659, 143660, 143661, 143662, 143663, 143664, 143665, 143666, 143667, 143668, 143669, 143670, 143671, 143672, 143673, 143674 were selected from the ORFs having the nucleotide sequence represented by SEQ ID NO: 80776

The probes having the nucleotide sequence represented by SEQ ID NOS: 143675, 143676, 143677, 143678, 143679, 143680, 143681, 143682, 143683, 143684, 143685 were selected from the ORFs having the nucleotide sequence represented by SEQ ID NO: 80777

The probes having the nucleotide sequence represented by SEQ ID NOS: 143686, 143687, 143688, 143689, 143690, 143691, 143692, 143693, 143694, 143695, 143696 were selected from the ORFs having the nucleotide sequence represented by SEQ ID NO: 80778

The probes having the nucleotide sequence represented by SEQ ID NOS: 143697, 143698, 143699, 143700, 143701, 143702, 143703, 143704, 143705, 143706, 143707 were selected from the ORFs having the nucleotide sequence represented by SEQ ID NO: 80779

The probes having the nucleotide sequence represented by SEQ ID NOS: 143708, 143709, 143710, 143711, 143712, 143713, 143714, 143715, 143716, 143717, 143718 were selected from the ORFs having the nucleotide sequence represented by SEQ ID NO: 80780

The probes having the nucleotide sequence represented by SEQ ID NOS: 143719, 143720, 143721, 143722, 143723, 143724, 143725, 143726, 143727, 143728, 143729 were selected from the ORFs having the nucleotide sequence represented by SEQ ID NO: 80781

The probes having the nucleotide sequence represented by SEQ ID NOS: 143730, 143731, 143732, 143733, 143734, 143735, 143736, 143737, 143738, 143739, 143740 were selected from the ORFs having the nucleotide sequence represented by SEQ ID NO: 80782

The probes having the nucleotide sequence represented by SEQ ID NOS: 143741, 143742, 143743, 143744, 143745, 143746, 143747, 143748, 143749, 143750, 143751 were selected from the ORFs having the nucleotide sequence represented by SEQ ID NO: 80783

The probes having the nucleotide sequence represented by SEQ ID NOS: 143752, 143753, 143754, 143755, 143756, 143757, 143758, 143759, 143760, 143761, 143762 were selected from the ORFs having the nucleotide sequence represented by SEQ ID NO: 80784

The probes having the nucleotide sequence represented by SEQ ID NOS: 143763, 143764, 143765, 143766, 143767, 143768, 143769, 143770, 143771, 143772, 143773 were selected from the ORFs having the nucleotide sequence represented by SEQ ID NO: 80785

The probes having the nucleotide sequence represented by SEQ ID NOS: 143774, 143775, 143776, 143777, 143778, 143779, 143780, 143781, 143782, 143783, 143784 were selected from the ORFs having the nucleotide sequence represented by SEQ ID NO: 80786.

The probes having the nucleotide sequence represented by SEQ ID NOS: 143785, 143786, 143787, 143788, 143789, 143790, 143791, 143792, 143793, 143794, 143795 were selected from the ORFs having the nucleotide sequence represented by SEQ ID NO: 80787

The probes having the nucleotide sequence represented by SEQ ID NOS: 143796, 143797, 143798, 143799, 143800, 143801, 143802, 143803, 143804, 143805, 143806, 143807, 143808, 143809, 143810, 143811, 143812, 143813, 143814, 143815, 143816, 143817 were selected from the ORFs having the nucleotide sequence represented by SEQ ID NO: 80788

The probes having the nucleotide sequence represented by SEQ ID NOS: 143818, 143819, 143820, 143821, 143822, 143823, 143824, 143825, 143826, 143827, 143828 were selected from the ORFs having the nucleotide sequence represented by SEQ ID NO: 80789

The probes having the nucleotide sequence represented by SEQ ID NOS: 143829, 143830, 143831, 143832, 143833, 143834, 143835, 143836, 143837, 143838, 143839 were selected from the ORFs having the nucleotide sequence represented by SEQ ID NO: 80790

The probes having the nucleotide sequence represented by SEQ ID NOS: 143840, 143841, 143842, 143843, 143844, 143845, 143846, 143847, 143848, 143849, 143850 were selected from the ORFs having the nucleotide sequence represented by SEQ ID NO: 80791

The probes having the nucleotide sequence represented by SEQ ID NOS: 143851, 143852, 143853, 143854, 143855, 143856, 143857, 143858, 143859, 143860, 143861 were selected from the ORFs having the nucleotide sequence represented by SEQ ID NO: 80792

The probes having the nucleotide sequence represented by SEQ ID NOS: 143862, 143863, 143864, 143865, 143866, 143867, 143868, 143869, 143870, 143871, 143872 were selected from the ORFs having the nucleotide sequence represented by SEQ ID NO: 80793

The probes having the nucleotide sequence represented by SEQ ID NOS: 143873, 143874, 143875, 143876, 143877, 143878, 143879, 143880, 143881, 143882, 143883 were selected from the ORFs having the nucleotide sequence represented by SEQ ID NO: 80794

The probes having the nucleotide sequence represented by SEQ ID NOS: 143884, 143885, 143886, 143887, 143888, 143889, 143890, 143891, 143892, 143893, 143894 were selected from the ORFs having the nucleotide sequence represented by SEQ ID NO: 80795

The probes having the nucleotide sequence represented by SEQ ID NOS: 143895, 143896, 143897, 143898, 143899, 143900, 143901, 143902, 143903, 143904, 143905 were selected from the ORFs having the nucleotide sequence represented by SEQ ID NO: 80796

The probes having the nucleotide sequence represented by SEQ ID NOS: 143906, 143907, 143908, 143909, 143910, 143911, 143912, 143913, 143914, 143915, 143916 were selected from the ORFs having the nucleotide sequence represented by SEQ ID NO: 80797.

The probes having the nucleotide sequence represented by SEQ ID NOS: 143917, 143918, 143919, 143920, 143921, 143922, 143923, 143924, 143925, 143926, 143927 were selected from the ORFs having the nucleotide sequence represented by SEQ ID NO: 80798

The probes having the nucleotide sequence represented by SEQ ID NOS: 143928, 143929, 143930, 143931, 143932, 143933, 143934, 143935, 143936, 143937, 143938 were selected from the ORFs having the nucleotide sequence represented by SEQ ID NO: 80799

The probes having the nucleotide sequence represented by SEQ ID NOS: 143939, 143940, 143941, 143942, 143943, 143944, 143945, 143946, 143947, 143948, 143949 were selected from the ORFs having the nucleotide sequence represented by SEQ ID NO: 80800

The probes having the nucleotide sequence represented by SEQ ID NOS: 143950, 143951, 143952, 143953, 143954, 143955, 143956, 143957, 143958, 143959, 143960 were selected from the ORFs having the nucleotide sequence represented by SEQ ID NO: 80801

The probes having the nucleotide sequence represented by SEQ ID NOS: 143961, 143962, 143963, 143964, 143965, 143966, 143967, 143968, 143969, 143970, 143971 were selected from the ORFs having the nucleotide sequence represented by SEQ ID NO: 80802

The probes having the nucleotide sequence represented by SEQ ID NOS: 143972, 143973, 143974, 143975, 143976, 143977, 143978, 143979, 143980, 143981, 143982 were selected from the ORFs having the nucleotide sequence represented by SEQ ID NO: 80803

The probes having the nucleotide sequence represented by SEQ ID NOS: 143983, 143984, 143985, 143986, 143987, 143988, 143989, 143990, 143991, 143992, 143993 were selected from the ORFs having the nucleotide sequence represented by SEQ ID NO: 80804

The probes having the nucleotide sequence represented by SEQ ID NOS: 143994, 143995, 143996, 143997, 143998, 143999, 144000, 144001, 144002, 144003, 144004 were selected from the ORFs having the nucleotide sequence represented by SEQ ID NO: 80805

The probes having the nucleotide sequence represented by SEQ ID NOS: 144005, 144006, 144007, 144008, 144009, 144010, 144011, 144012, 144013, 144014, 144015 were selected from the ORFs having the nucleotide sequence represented by SEQ ID NO: 80806

The probes having the nucleotide sequence represented by SEQ ID NOS: 144016, 144017, 144018, 144019, 144020, 144021, 144022, 144023, 144024, 144025, 144026 were selected from the ORFs having the nucleotide sequence represented by SEQ ID NO: 80807

The probes having the nucleotide sequence represented by SEQ ID NOS: 144027, 144028, 144029, 144030, 144031, 144032, 144033, 144034, 144035, 144036, 144037 were selected from the ORFs having the nucleotide sequence represented by SEQ ID NO: 80808.

The probes having the nucleotide sequence represented by SEQ ID NOS: 144038, 144039, 144040, 144041, 144042, 144043, 144044, 144045, 144046, 144047, 144048 were selected from the ORFs having the nucleotide sequence represented by SEQ ID NO: 80809

The probes having the nucleotide sequence represented by SEQ ID NOS: 144049, 144050, 144051, 144052, 144053, 144054, 144055, 144056, 144057, 144058, 144059 were selected from the ORFs having the nucleotide sequence represented by SEQ ID NO: 80810

The probes having the nucleotide sequence represented by SEQ ID NOS: 144060, 144061, 144062, 144063, 144064, 144065, 144066, 144067, 144068, 144069, 144070 were selected from the ORFs having the nucleotide sequence represented by SEQ ID NO: 80811

The probes having the nucleotide sequence represented by SEQ ID NOS: 144071, 144072, 144073, 144074, 144075, 144076, 144077, 144078, 144079, 144080, 144081 were selected from the ORFs having the nucleotide sequence represented by SEQ ID NO: 80812

The probes having the nucleotide sequence represented by SEQ ID NOS: 144082, 144083, 144084, 144085, 144086, 144087, 144088, 144089, 144090, 144091, 144092 were selected from the ORFs having the nucleotide sequence represented by SEQ ID NO: 80813

The probes having the nucleotide sequence represented by SEQ ID NOS: 144093, 144094, 144095, 144096, 144097, 144098, 144099, 144100, 144101, 144102, 144103 were selected from the ORFs having the nucleotide sequence represented by SEQ ID NO: 80814

The probes having the nucleotide sequence represented by SEQ ID NOS: 144104, 144105, 144106, 144107, 144108, 144109, 144110, 144111, 144112, 144113, 144114 were selected from the ORFs having the nucleotide sequence represented by SEQ ID NO: 80815

The probes having the nucleotide sequence represented by SEQ ID NOS: 144115, 144116, 144117, 144118, 144119, 144120, 144121, 144122, 144123, 144124, 144125 were selected from the ORFs having the nucleotide sequence represented by SEQ ID NO: 80816

The probes having the nucleotide sequence represented by SEQ ID NOS: 144126, 144127, 144128, 144129, 144130, 144131, 144132, 144133, 144134, 144135, 144136 were selected from the ORFs having the nucleotide sequence represented by SEQ ID NO: 80817

The probes having the nucleotide sequence represented by SEQ ID NOS: 144137, 144138, 144139, 144140, 144141, 144142, 144143, 144144, 144145, 144146, 144147 were selected from the ORFs having the nucleotide sequence represented by SEQ ID NO: 80818

The probes having the nucleotide sequence represented by SEQ ID NOS: 144148, 144149, 144150, 144151, 144152, 144153, 144154, 144155, 144156, 144157, 144158, 144159, 144160, 144161, 144162, 144163, 144164, 144165, 144166, 144167, 144168, 144169 were selected from the ORFs having the nucleotide sequence represented by SEQ ID NO: 80819.

The probes having the nucleotide sequence represented by SEQ ID NOS: 144170, 144171, 144172, 144173, 144174, 144175, 144176, 144177, 144178, 144179, 144180 were selected from the ORFs having the nucleotide sequence represented by SEQ ID NO: 80820

The probes having the nucleotide sequence represented by SEQ ID NOS: 144181, 144182, 144183, 144184, 144185, 144186, 144187, 144188, 144189, 144190, 144191 were selected from the ORFs having the nucleotide sequence represented by SEQ ID NO: 80821

The probes having the nucleotide sequence represented by SEQ ID NOS: 144192, 144193, 144194, 144195, 144196, 144197, 144198, 144199, 144200, 144201, 144202 were selected from the ORFs having the nucleotide sequence represented by SEQ ID NO: 80822

The probes having the nucleotide sequence represented by SEQ ID NOS: 144203, 144204, 144205, 144206, 144207, 144208, 144209, 144210, 144211, 144212, 144213 were selected from the ORFs having the nucleotide sequence represented by SEQ ID NO: 80823

The probes having the nucleotide sequence represented by SEQ ID NOS: 144214, 144215, 144216, 144217, 144218, 144219, 144220, 144221, 144222, 144223, 144224 were selected from the ORFs having the nucleotide sequence represented by SEQ ID NO: 80824

The probes having the nucleotide sequence represented by SEQ ID NOS: 144225, 144226, 144227, 144228, 144229, 144230, 144231, 144232, 144233, 144234, 144235 were selected from the ORFs having the nucleotide sequence represented by SEQ ID NO: 80825

The probes having the nucleotide sequence represented by SEQ ID NOS: 144236, 144237, 144238, 144239, 144240, 144241, 144242, 144243, 144244, 144245, 144246 were selected from the ORFs having the nucleotide sequence represented by SEQ ID NO: 80826

The probes having the nucleotide sequence represented by SEQ ID NOS: 144247, 144248, 144249, 144250, 144251, 144252, 144253, 144254, 144255, 144256, 144257 were selected from the ORFs having the nucleotide sequence represented by SEQ ID NO: 80827

The probes having the nucleotide sequence represented by SEQ ID NOS: 144258, 144259, 144260, 144261, 144262, 144263, 144264, 144265, 144266, 144267, 144268 were selected from the ORFs having the nucleotide sequence represented by SEQ ID NO: 80828

The probes having the nucleotide sequence represented by SEQ ID NOS: 144269, 144270, 144271, 144272, 144273, 144274, 144275, 144276, 144277, 144278, 144279 were selected from the ORFs having the nucleotide sequence represented by SEQ ID NO: 80829

The probes having the nucleotide sequence represented by SEQ ID NOS: 144280, 144281, 144282, 144283, 144284, 144285, 144286, 144287, 144288, 144289, 144290 were selected from the ORFs having the nucleotide sequence represented by SEQ ID NO: 80830.

The probes having the nucleotide sequence represented by SEQ ID NOS: 144291, 144292, 144293, 144294, 144295, 144296, 144297, 144298, 144299, 144300, 144301 were selected from the ORFs having the nucleotide sequence represented by SEQ ID NO: 80831

The probes having the nucleotide sequence represented by SEQ ID NOS: 144302, 144303, 144304, 144305, 144306, 144307, 144308, 144309, 144310, 144311, 144312 were selected from the ORFs having the nucleotide sequence represented by SEQ ID NO: 80832

The probes having the nucleotide sequence represented by SEQ ID NOS: 144313, 144314, 144315, 144316, 144317, 144318, 144319, 144320, 144321, 144322, 144323 were selected from the ORFs having the nucleotide sequence represented by SEQ ID NO: 80833

The probes having the nucleotide sequence represented by SEQ ID NOS: 144324, 144325, 144326, 144327, 144328, 144329, 144330, 144331, 144332, 144333, 144334 were selected from the ORFs having the nucleotide sequence represented by SEQ ID NO: 80834

The probes having the nucleotide sequence represented by SEQ ID NOS: 144335, 144336, 144337, 144338, 144339, 144340, 144341, 144342, 144343, 144344, 144345 were selected from the ORFs having the nucleotide sequence represented by SEQ ID NO: 80835

The probes having the nucleotide sequence represented by SEQ ID NOS: 144346, 144347, 144348, 144349, 144350, 144351, 144352, 144353, 144354, 144355, 144356 were selected from the ORFs having the nucleotide sequence represented by SEQ ID NO: 80836

The probes having the nucleotide sequence represented by SEQ ID NOS: 144357, 144358, 144359, 144360, 144361, 144362, 144363, 144364, 144365, 144366, 144367 were selected from the ORFs having the nucleotide sequence represented by SEQ ID NO: 80837

The probes having the nucleotide sequence represented by SEQ ID NOS: 144368, 144369, 144370, 144371, 144372, 144373, 144374, 144375, 144376, 144377, 144378 were selected from the ORFs having the nucleotide sequence represented by SEQ ID NO: 80838

The probes having the nucleotide sequence represented by SEQ ID NOS: 144379, 144380, 144381, 144382, 144383, 144384, 144385, 144386, 144387, 144388, 144389 were selected from the ORFs having the nucleotide sequence represented by SEQ ID NO: 80839

The probes having the nucleotide sequence represented by SEQ ID NOS: 144390, 144391, 144392, 144393, 144394, 144395, 144396, 144397, 144398, 144399, 144400 were selected from the ORFs having the nucleotide sequence represented by SEQ ID NO: 80840

The probes having the nucleotide sequence represented by SEQ ID NOS: 144401, 144402, 144403, 144404, 144405, 144406, 144407, 144408, 144409, 144410, 144411 were selected from the ORFs having the nucleotide sequence represented by SEQ ID NO: 80841.

The probes having the nucleotide sequence represented by SEQ ID NOS: 144412, 144413, 144414, 144415, 144416, 144417, 144418, 144419, 144420, 144421, 144422 were selected from the ORFs having the nucleotide sequence represented by SEQ ID NO: 80842

The probes having the nucleotide sequence represented by SEQ ID NOS: 144423, 144424, 144425, 144426, 144427, 144428, 144429, 144430, 144431, 144432, 144433 were selected from the ORFs having the nucleotide sequence represented by SEQ ID NO: 80843

The probes having the nucleotide sequence represented by SEQ ID NOS: 144434, 144435, 144436, 144437, 144438, 144439, 144440, 144441, 144442, 144443, 144444 were selected from the ORFs having the nucleotide sequence represented by SEQ ID NO: 80844

The probes having the nucleotide sequence represented by SEQ ID NOS: 144445, 144446, 144447, 144448, 144449, 144450, 144451, 144452, 144453, 144454, 144455 were selected from the ORFs having the nucleotide sequence represented by SEQ ID NO: 80845

The probes having the nucleotide sequence represented by SEQ ID NOS: 144456, 144457, 144458, 144459, 144460, 144461, 144462, 144463, 144464, 144465, 144466 were selected from the ORFs having the nucleotide sequence represented by SEQ ID NO: 80846

The probes having the nucleotide sequence represented by SEQ ID NOS: 144467, 144468, 144469, 144470, 144471, 144472, 144473, 144474, 144475, 144476, 144477 were selected from the ORFs having the nucleotide sequence represented by SEQ ID NO: 80847

The probes having the nucleotide sequence represented by SEQ ID NOS: 144478, 144479, 144480, 144481, 144482, 144483, 144484, 144485, 144486, 144487, 144488 were selected from the ORFs having the nucleotide sequence represented by SEQ ID NO: 80848

The probes having the nucleotide sequence represented by SEQ ID NOS: 144489, 144490, 144491, 144492, 144493, 144494, 144495, 144496, 144497, 144498, 144499 were selected from the ORFs having the nucleotide sequence represented by SEQ ID NO: 80849

The probes having the nucleotide sequence represented by SEQ ID NOS: 144500, 144501, 144502, 144503, 144504, 144505, 144506, 144507, 144508, 144509, 144510 were selected from the ORFs having the nucleotide sequence represented by SEQ ID NO: 80850

The probes having the nucleotide sequence represented by SEQ ID NOS: 144511, 144512, 144513, 144514, 144515, 144516, 144517, 144518, 144519, 144520, 144521 were selected from the ORFs having the nucleotide sequence represented by SEQ ID NO: 80851

The probes having the nucleotide sequence represented by SEQ ID NOS: 144522, 144523, 144524, 144525, 144526, 144527, 144528, 144529, 144530, 144531, 144532 were selected from the ORFs having the nucleotide sequence represented by SEQ ID NO: 80852.

The probes having the nucleotide sequence represented by SEQ ID NOS: 144533, 144534, 144535, 144536, 144537, 144538, 144539, 144540, 144541, 144542, 144543 were selected from the ORFs having the nucleotide sequence represented by SEQ ID NO: 80853.

The probes having the nucleotide sequence represented by SEQ ID NOS: 144544, 144545, 144546, 144547, 144548, 144549, 144550, 144551, 144552, 144553, 144554 were selected from the ORFs having the nucleotide sequence represented by SEQ ID NO: 80854.

The probes having the nucleotide sequence represented by SEQ ID NOS: 144555, 144556, 144557, 144558, 144559, 144560, 144561, 144562, 144563, 144564, 144565 were selected from the ORFs having the nucleotide sequence represented by SEQ ID NO: 80855.

The probes having the nucleotide sequence represented by SEQ ID NOS: 144566, 144567, 144568, 144569, 144570, 144571, 144572, 144573, 144574, 144575, 144576 were selected from the ORFs having the nucleotide sequence represented by SEQ ID NO: 80856.

The probes having the nucleotide sequence represented by SEQ ID NOS: 144577, 144578, 144579, 144580, 144581, 144582, 144583, 144584, 144585, 144586, 144587 were selected from the ORFs having the nucleotide sequence represented by SEQ ID NO: 80857.

The probes having the nucleotide sequence represented by SEQ ID NOS: 144588, 144589, 144590, 144591, 144592, 144593, 144594, 144595, 144596, 144597, 144598 were selected from the ORFs having the nucleotide sequence represented by SEQ ID NO: 80858.

The probes having the nucleotide sequence represented by SEQ ID NOS: 144599, 144600, 144601, 144602, 144603, 144604, 144605, 144606, 144607, 144608, 144609 were selected from the ORFs having the nucleotide sequence represented by SEQ ID NO: 80859.

The probes having the nucleotide sequence represented by SEQ ID NOS: 144610, 144611, 144612, 144613, 144614, 144615, 144616, 144617, 144618, 144619, 144620 were selected from the ORFs having the nucleotide sequence represented by SEQ ID NO: 80860.

The probes having the nucleotide sequence represented by SEQ ID NOS: 144621, 144622, 144623, 144624, 144625, 144626, 144627, 144628, 144629, 144630, 144631 were selected from the ORFs having the nucleotide sequence represented by SEQ ID NO: 80861.

The probes having the nucleotide sequence represented by SEQ ID NOS: 144632, 144633, 144634, 144635, 144636, 144637, 144638, 144639, 144640, 144641, 144642 were selected from the ORFs having the nucleotide sequence represented by SEQ ID NO: 80862.

The probes having the nucleotide sequence represented by SEQ ID NOS: 144643, 144644, 144645, 144646, 144647, 144648, 144649, 144650, 144651, 144652, 144653 were selected from the ORFs having the nucleotide sequence represented by SEQ ID NO: 80863.

The probes having the nucleotide sequence represented by SEQ ID NOS: 144654, 144655, 144656, 144657, 144658, 144659, 144660, 144661, 144662, 144663, 144664 were selected from the ORFs having the nucleotide sequence represented by SEQ ID NO: 80864.

The probes having the nucleotide sequence represented by SEQ ID NOS: 144665, 144666, 144667, 144668, 144669, 144670, 144671, 144672, 144673, 144674, 144675 were selected from the ORFs having the nucleotide sequence represented by SEQ ID NO: 80865.

The probes having the nucleotide sequence represented by SEQ ID NOS: 144676, 144677, 144678, 144679, 144680, 144681, 144682, 144683, 144684, 144685, 144686 were selected from the ORFs having the nucleotide sequence represented by SEQ ID NO: 80866.

The probes having the nucleotide sequence represented by SEQ ID NOS: 144687, 144688, 144689, 144690, 144691, 144692, 144693, 144694, 144695, 144696, 144697 were selected from the ORFs having the nucleotide sequence represented by SEQ ID NO: 80867.

The probes having the nucleotide sequence represented by SEQ ID NOS: 144698, 144699, 144700, 144701, 144702, 144703, 144704, 144705, 144706, 144707, 144708 were selected from the ORFs having the nucleotide sequence represented by SEQ ID NO: 80868.

The probes having the nucleotide sequence represented by SEQ ID NOS: 144709, 144710, 144711, 144712, 144713, 144714, 144715, 144716, 144717, 144718, 144719 were selected from the ORFs having the nucleotide sequence represented by SEQ ID NO: 80869.

The probes having the nucleotide sequence represented by SEQ ID NOS: 144720, 144721, 144722, 144723, 144724, 144725, 144726, 144727, 144728, 144729, 144730 were selected from the ORFs having the nucleotide sequence represented by SEQ ID NO: 80870.

The probes having the nucleotide sequence represented by SEQ ID NOS: 144731, 144732, 144733, 144734, 144735, 144736, 144737, 144738, 144739, 144740, 144741 were selected from the ORFs having the nucleotide sequence represented by SEQ ID NO: 80871.

The probes having the nucleotide sequence represented by SEQ ID NOS: 144742, 144743, 144744, 144745, 144746, 144747, 144748, 144749, 144750, 144751, 144752 were selected from the ORFs having the nucleotide sequence represented by SEQ ID NO: 80872.

The probes having the nucleotide sequence represented by SEQ ID NOS: 144753, 144754, 144755, 144756, 144757, 144758, 144759, 144760, 144761, 144762, 144763 were selected from the ORFs having the nucleotide sequence represented by SEQ ID NO: 80873.

The probes having the nucleotide sequence represented by SEQ ID NOS: 144764, 144765, 144766, 144767, 144768, 144769, 144770, 144771, 144772, 144773, 144774 were selected from the ORFs having the nucleotide sequence represented by SEQ ID NO: 80874.

The probes having the nucleotide sequence represented by SEQ ID NOS: 144775, 144776, 144777, 144778, 144779, 144780, 144781, 144782, 144783, 144784, 144785 were selected from the ORFs having the nucleotide sequence represented by SEQ ID NO: 80875.

The probes having the nucleotide sequence represented by SEQ ID NOS: 144786, 144787, 144788, 144789, 144790, 144791, 144792, 144793, 144794, 144795, 144796 were selected from the ORFs having the nucleotide sequence represented by SEQ ID NO: 80876.

The probes having the nucleotide sequence represented by SEQ ID NOS: 144797, 144798, 144799, 144800, 144801, 144802, 144803, 144804, 144805, 144806, 144807 were selected from the ORFs having the nucleotide sequence represented by SEQ ID NO: 80877.

The probes having the nucleotide sequence represented by SEQ ID NOS: 144808, 144809, 144810, 144811, 144812, 144813, 144814, 144815, 144816, 144817, 144818 were selected from the ORFs having the nucleotide sequence represented by SEQ ID NO: 80878.

The probes having the nucleotide sequence represented by SEQ ID NOS: 144819, 144820, 144821, 144822, 144823, 144824, 144825, 144826, 144827, 144828, 144829 were selected from the ORFs having the nucleotide sequence represented by SEQ ID NO: 80879.

The probes having the nucleotide sequence represented by SEQ ID NOS: 144830, 144831, 144832, 144833, 144834, 144835, 144836, 144837, 144838, 144839, 144840 were selected from the ORFs having the nucleotide sequence represented by SEQ ID NO: 80880.

The probes having the nucleotide sequence represented by SEQ ID NOS: 144841, 144842, 144843, 144844, 144845, 144846, 144847, 144848, 144849, 144850, 144851 were selected from the ORFs having the nucleotide sequence represented by SEQ ID NO: 80881.

The probes having the nucleotide sequence represented by SEQ ID NOS: 144852, 144853, 144854, 144855, 144856, 144857, 144858, 144859, 144860, 144861, 144862 were selected from the ORFs having the nucleotide sequence represented by SEQ ID NO: 80882.

The probes having the nucleotide sequence represented by SEQ ID NOS: 144863, 144864, 144865, 144866, 144867, 144868, 144869, 144870, 144871, 144872, 144873 were selected from the ORFs having the nucleotide sequence represented by SEQ ID NO: 80883.

The probes having the nucleotide sequence represented by SEQ ID NOS: 144874, 144875, 144876, 144877, 144878, 144879, 144880, 144881, 144882, 144883, 144884 were selected from the ORFs having the nucleotide sequence represented by SEQ ID NO: 80884.

The probes having the nucleotide sequence represented by SEQ ID NOS: 144885, 144886, 144887, 144888, 144889, 144890, 144891, 144892, 144893, 144894, 144895 were selected from the ORFs having the nucleotide sequence represented by SEQ ID NO: 80885.

The probes having the nucleotide sequence represented by SEQ ID NOS: 144896, 144897, 144898, 144899, 144900, 144901, 144902, 144903, 144904, 144905, 144906 were selected from the ORFs having the nucleotide sequence represented by SEQ ID NO: 80886.

The probes having the nucleotide sequence represented by SEQ ID NOS: 144907, 144908, 144909, 144910, 144911, 144912, 144913, 144914, 144915, 144916, 144917 were selected from the ORFs having the nucleotide sequence represented by SEQ ID NO: 80887.

The probes having the nucleotide sequence represented by SEQ ID NOS: 144918, 144919, 144920, 144921, 144922, 144923, 144924, 144925, 144926, 144927, 144928 were selected from the ORFs having the nucleotide sequence represented by SEQ ID NO: 80888.

The probes having the nucleotide sequence represented by SEQ ID NOS: 144929, 144930, 144931, 144932, 144933, 144934, 144935, 144936, 144937, 144938, 144939 were selected from the ORFs having the nucleotide sequence represented by SEQ ID NO: 80889.

The probes having the nucleotide sequence represented by SEQ ID NOS: 144940, 144941, 144942, 144943, 144944, 144945, 144946, 144947, 144948, 144949, 144950 were selected from the ORFs having the nucleotide sequence represented by SEQ ID NO: 80890.

The probes having the nucleotide sequence represented by SEQ ID NOS: 144951, 144952, 144953, 144954, 144955, 144956, 144957, 144958, 144959, 144960, 144961 were selected from the ORFs having the nucleotide sequence represented by SEQ ID NO: 80891.

The probes having the nucleotide sequence represented by SEQ ID NOS: 144962, 144963, 144964, 144965, 144966, 144967, 144968, 144969, 144970, 144971, 144972 were selected from the ORFs having the nucleotide sequence represented by SEQ ID NO: 80892.

The probes having the nucleotide sequence represented by SEQ ID NOS: 144973, 144974, 144975, 144976, 144977, 144978, 144979, 144980, 144981, 144982, 144983 were selected from the ORFs having the nucleotide sequence represented by SEQ ID NO: 80893.

The probes having the nucleotide sequence represented by SEQ ID NOS: 144984, 144985, 144986, 144987, 144988, 144989, 144990, 144991, 144992, 144993, 144994, 144995, 144996, 144997, 144998, 144999, 145000, 145001, 145002, 145003, 145004, 145005 were selected from the ORFs having the nucleotide sequence represented by SEQ ID NO: 80894.

The probes having the nucleotide sequence represented by SEQ ID NOS: 145006, 145007, 145008, 145009, 145010, 145011, 145012, 145013, 145014, 145015, 145016 were selected from the ORFs having the nucleotide sequence represented by SEQ ID NO: 80895.

The probes having the nucleotide sequence represented by SEQ ID NOS: 145017, 145018, 145019, 145020, 145021, 145022, 145023, 145024, 145025, 145026, 145027 were selected from the ORFs having the nucleotide sequence represented by SEQ ID NO: 80896.

The probes having the nucleotide sequence represented by SEQ ID NOS: 145028, 145029, 145030, 145031, 145032, 145033, 145034, 145035, 145036, 145037, 145038 were selected from the ORFs having the nucleotide sequence represented by SEQ ID NO: 80897.

The probes having the nucleotide sequence represented by SEQ ID NOS: 145039, 145040, 145041, 145042, 145043, 145044, 145045, 145046, 145047, 145048, 145049 were selected from the ORFs having the nucleotide sequence represented by SEQ ID NO: 80898.

The probes having the nucleotide sequence represented by SEQ ID NOS: 145050, 145051, 145052, 145053, 145054, 145055, 145056, 145057, 145058, 145059, 145060 were selected from the ORFs having the nucleotide sequence represented by SEQ ID NO: 80899.

The probes having the nucleotide sequence represented by SEQ ID NOS: 145061, 145062, 145063, 145064, 145065, 145066, 145067, 145068, 145069, 145070, 145071 were selected from the ORFs having the nucleotide sequence represented by SEQ ID NO: 80900.

The probes having the nucleotide sequence represented by SEQ ID NOS: 145072, 145073, 145074, 145075, 145076, 145077, 145078, 145079, 145080, 145081, 145082 were selected from the ORFs having the nucleotide sequence represented by SEQ ID NO: 80901.

The probes having the nucleotide sequence represented by SEQ ID NOS: 145083, 145084, 145085, 145086, 145087, 145088, 145089, 145090, 145091, 145092, 145093 were selected from the ORFs having the nucleotide sequence represented by SEQ ID NO: 80902.

The probes having the nucleotide sequence represented by SEQ ID NOS: 145094, 145095, 145096, 145097, 145098, 145099, 145100, 145101, 145102, 145103, 145104 were selected from the ORFs having the nucleotide sequence represented by SEQ ID NO: 80903.

The probes having the nucleotide sequence represented by SEQ ID NOS: 14510.5, 145106, 145107, 145108, 145109, 145110, 145111, 145112, 145113, 145114, 145115 were selected from the ORFs having the nucleotide sequence represented by SEQ ID NO: 80904.

The probes having the nucleotide sequence represented by SEQ ID NOS: 145116, 145117, 145118, 145119, 145120, 145121, 145122, 145123, 145124, 145125, 145126 were selected from the ORFs having the nucleotide sequence represented by SEQ ID NO: 80905.

The probes having the nucleotide sequence represented by SEQ ID NOS: 145127, 145128, 145129, 145130, 145131, 145132, 145133, 145134, 145135, 145136, 145137 were selected from the ORFs having the nucleotide sequence represented by SEQ ID NO: 80906.

The probes having the nucleotide sequence represented by SEQ ID NOS: 145138, 145139, 145140, 145141, 145142, 145143, 145144, 145145, 145146, 145147, 145148 were selected from the ORFs having the nucleotide sequence represented by SEQ ID NO: 80907.

The probes having the nucleotide sequence represented by SEQ ID NOS: 145149, 145150, 145151, 145152, 145153, 145154, 145155, 145156, 145157, 145158, 145159 were selected from the ORFs having the nucleotide sequence represented by SEQ ID NO: 80908.

The probes having the nucleotide sequence represented by SEQ ID NOS: 145160, 145161, 145162, 145163, 145164, 145165, 145166, 145167, 145168, 145169, 145170 were selected from the ORFs having the nucleotide sequence represented by SEQ ID NO: 80909.

The probes having the nucleotide sequence represented by SEQ ID NOS: 145171, 145172, 145173, 145174, 145175, 145176, 145177, 145178, 145179, 145180, 145181 were selected from the ORFs having the nucleotide sequence represented by SEQ ID NO: 80910.

The probes having the nucleotide sequence represented by SEQ ID NOS: 145182, 145183, 145184, 145185, 145186, 145187, 145188, 145189, 145190, 145191, 145192 were selected from the ORFs having the nucleotide sequence represented by SEQ ID NO: 80911.

The probes having the nucleotide sequence represented by SEQ ID NOS: 145193, 145194, 145195, 145196, 145197, 145198, 145199, 145200, 145201, 145202, 145203 were selected from the ORFs having the nucleotide sequence represented by SEQ ID NO: 80912.

The probes having the nucleotide sequence represented by SEQ ID NOS: 145204, 145205, 145206, 145207, 145208, 145209, 145210, 145211, 145212, 145213, 145214 were selected from the ORFs having the nucleotide sequence represented by SEQ ID NO: 80913.

The probes having the nucleotide sequence represented by SEQ ID NOS: 145215, 145216, 145217, 145218, 145219, 145220, 145221, 145222, 145223, 145224, 145225 were selected from the ORFs having the nucleotide sequence represented by SEQ ID NO: 80914.

The probes having the nucleotide sequence represented by SEQ ID NOS: 145226, 145227, 145228, 145229, 145230, 145231, 145232, 145233, 145234, 145235, 145236 were selected from the ORFs having the nucleotide sequence represented by SEQ ID NO: 80915.

The probes having the nucleotide sequence represented by SEQ ID NOS: 145237, 145238, 145239, 145240, 145241, 145242, 145243, 145244, 145245, 145246, 145247 were selected from the ORFs having the nucleotide sequence represented by SEQ ID NO: 80916.

The probes having the nucleotide sequence represented by SEQ ID NOS: 145248, 145249, 145250, 145251, 145252, 145253, 145254, 145255, 145256, 145257, 145258 were selected from the ORFs having the nucleotide sequence represented by SEQ ID NO: 80917.

The probes having the nucleotide sequence represented by SEQ ID NOS: 145259, 145260, 145261, 145262, 145263, 145264, 145265, 145266, 145267, 145268, 145269 were selected from the ORFs having the nucleotide sequence represented by SEQ ID NO: 80918.

The probes having the nucleotide sequence represented by SEQ ID NOS: 145270, 145271, 145272, 145273, 145274, 145275, 145276, 145277, 145278, 145279, 145280 were selected from the ORFs having the nucleotide sequence represented by SEQ ID NO: 80919.

The probes having the nucleotide sequence represented by SEQ ID NOS: 145281, 145282, 145283, 145284, 145285, 145286, 145287, 145288, 145289, 145290, 145291 were selected from the ORFs having the nucleotide sequence represented by SEQ ID NO: 80920.

The probes having the nucleotide sequence represented by SEQ ID NOS: 145292, 145293, 145294, 145295, 145296, 145297, 145298, 145299, 145300, 145301, 145302 were selected from the ORFs having the nucleotide sequence represented by SEQ ID NO: 80921.

The probes having the nucleotide sequence represented by SEQ ID NOS: 145303, 145304, 145305, 145306, 145307, 145308, 145309, 145310, 145311, 145312, 145313 were selected from the ORFs having the nucleotide sequence represented by SEQ ID NO: 80922.

The probes having the nucleotide sequence represented by SEQ ID NOS: 145314, 145315, 145316, 145317, 145318, 145319, 145320, 145321, 145322, 145323, 145324 were selected from the ORFs having the nucleotide sequence represented by SEQ ID NO: 80923.

The probes having the nucleotide sequence represented by SEQ ID NOS: 145325, 145326, 145327, 145328, 145329, 145330, 145331, 145332, 145333, 145334, 145335 were selected from the ORFs having the nucleotide sequence represented by SEQ ID NO: 80924.

The probes having the nucleotide sequence represented by SEQ ID NOS: 145336, 145337, 145338, 145339, 145340, 145341, 145342, 145343, 145344, 145345, 145346 were selected from the ORFs having the nucleotide sequence represented by SEQ ID NO: 80925.

The probes having the nucleotide sequence represented by SEQ ID NOS: 145347, 145348, 145349, 145350, 145351, 145352, 145353, 145354, 145355, 145356, 145357 were selected from the ORFs having the nucleotide sequence represented by SEQ ID NO: 80926.

The probes having the nucleotide sequence represented by SEQ ID NOS: 145358, 145359, 145360, 145361, 145362, 145363, 145364, 145365, 145366, 145367, 145368 were selected from the ORFs having the nucleotide sequence represented by SEQ ID NO: 80927.

The probes having the nucleotide sequence represented by SEQ ID NOS: 145369, 145370, 145371, 145372, 145373, 145374, 145375, 145376, 145377, 145378, 145379 were selected from the ORFs having the nucleotide sequence represented by SEQ ID NO: 80928.

The probes having the nucleotide sequence represented by SEQ ID NOS: 145380, 145381, 145382, 145383, 145384, 145385, 145386, 145387, 145388, 145389, 145390 were selected from the ORFs having the nucleotide sequence represented by SEQ ID NO: 80929.

The probes having the nucleotide sequence represented by SEQ ID NOS: 145391, 145392, 145393, 145394, 145395, 145396, 145397, 145398, 145399, 145400, 145401 were selected from the ORFs having the nucleotide sequence represented by SEQ ID NO: 80930.

The probes having the nucleotide sequence represented by SEQ ID NOS: 145402, 145403, 145404, 145405, 145406, 145407, 145408, 145409, 145410, 145411, 145412 were selected from the ORFs having the nucleotide sequence represented by SEQ ID NO: 80931.

The probes having the nucleotide sequence represented by SEQ ID NOS: 145413, 145414, 145415, 145416, 145417, 145418, 145419, 145420, 145421, 145422, 145423 were selected from the ORFs having the nucleotide sequence represented by SEQ ID NO: 80932.

The probes having the nucleotide sequence represented by SEQ ID NOS: 145424, 145425, 145426, 145427, 145428, 145429, 145430, 145431, 145432, 145433, 145434 were selected from the ORFs having the nucleotide sequence represented by SEQ ID NO: 80933.

The probes having the nucleotide sequence represented by SEQ ID NOS: 145435, 145436, 145437, 145438, 145439, 145440, 145441, 145442, 145443, 145444, 145445 were selected from the ORFs having the nucleotide sequence represented by SEQ ID NO: 80934.

The probes having the nucleotide sequence represented by SEQ ID NOS: 145446, 145447, 145448, 145449, 145450, 145451, 145452, 145453, 145454, 145455, 145456 were selected from the ORFs having the nucleotide sequence represented by SEQ ID NO: 80935.

The probes having the nucleotide sequence represented by SEQ ID NOS: 145457, 145458, 145459, 145460, 145461, 145462, 145463, 145464, 145465, 145466, 145467 were selected from the ORFs having the nucleotide sequence represented by SEQ ID NO: 80936.

The probes having the nucleotide sequence represented by SEQ ID NOS: 145468, 145469, 145470, 145471, 145472, 145473, 145474, 145475, 145476, 145477, 145478 were selected from the ORFs having the nucleotide sequence represented by SEQ ID NO: 80937.

The probes having the nucleotide sequence represented by SEQ ID NOS: 145479, 145480, 145481, 145482, 145483, 145484, 145485, 145486, 145487, 145488, 145489 were selected from the ORFs having the nucleotide sequence represented by SEQ ID NO: 80938.

The probes having the nucleotide sequence represented by SEQ ID NOS: 145490, 145491, 145492, 145493, 145494, 145495, 145496, 145497, 145498, 145499, 145500 were selected from the ORFs having the nucleotide sequence represented by SEQ ID NO: 80939.

The probes having the nucleotide sequence represented by SEQ ID NOS: 145501, 145502, 145503, 145504, 145505, 145506, 145507, 145508, 145509, 145510, 145511 were selected from the ORFs having the nucleotide sequence represented by SEQ ID NO: 80940.

The probes having the nucleotide sequence represented by SEQ ID NOS: 145512, 145513, 145514, 145515, 145516, 145517, 145518, 145519, 145520, 145521, 145522 were selected from the ORFs having the nucleotide sequence represented by SEQ ID NO: 80941.

The probes having the nucleotide sequence represented by SEQ ID NOS: 145523, 145524, 145525, 145526, 145527, 145528, 145529, 145530, 145531, 145532, 145533 were selected from the ORFs having the nucleotide sequence represented by SEQ ID NO: 80942.

The probes having the nucleotide sequence represented by SEQ ID NOS: 145534, 145535, 145536, 145537, 145538, 145539, 145540, 145541, 145542, 145543, 145544 were selected from the ORFs having the nucleotide sequence represented by SEQ ID NO: 80943.

The probes having the nucleotide sequence represented by SEQ ID NOS: 145545, 145546, 145547, 145548, 145549, 145550, 145551, 145552, 145553, 145554, 145555 were selected from the ORFs having the nucleotide sequence represented by SEQ ID NO: 80944.

The probes having the nucleotide sequence represented by SEQ ID NOS: 145556, 145557, 145558, 145559, 145560, 145561, 145562, 145563, 145564, 145565, 145566 were selected from the ORFs having the nucleotide sequence represented by SEQ ID NO: 80945.

The probes having the nucleotide sequence represented by SEQ ID NOS: 145567, 145568, 145569, 145570, 145571, 145572, 145573, 145574, 145575, 145576, 145577 were selected from the ORFs having the nucleotide sequence represented by SEQ ID NO: 80946.

The probes having the nucleotide sequence represented by SEQ ID NOS: 145578, 145579, 145580, 145581, 145582, 145583, 145584, 145585, 145586, 145587, 145588 were selected from the ORFs having the nucleotide sequence represented by SEQ ID NO: 80947.

The probes having the nucleotide sequence represented by SEQ ID NOS: 145589, 145590, 145591, 145592, 145593, 145594, 145595, 145596, 145597, 145598, 145599 were selected from the ORFs having the nucleotide sequence represented by SEQ ID NO: 80948.

The probes having the nucleotide sequence represented by SEQ ID NOS: 145600, 145601, 145602, 145603, 145604, 145605, 145606, 145607, 145608, 145609, 145610 were selected from the ORFs having the nucleotide sequence represented by SEQ ID NO: 80949.

The probes having the nucleotide sequence represented by SEQ ID NOS: 145611, 145612, 145613, 145614, 145615, 145616, 145617, 145618, 145619, 145620, 145621 were selected from the ORFs having the nucleotide sequence represented by SEQ ID NO: 80950.

The probes having the nucleotide sequence represented by SEQ ID NOS: 145622, 145623, 145624, 145625, 145626, 145627, 145628, 145629, 145630, 145631, 145632 were selected from the ORFs having the nucleotide sequence represented by SEQ ID NO: 80951.

The probes having the nucleotide sequence represented by SEQ ID NOS: 145633, 145634, 145635, 145636, 145637, 145638, 145639, 145640, 145641, 145642, 145643 were selected from the ORFs having the nucleotide sequence represented by SEQ ID NO: 80952.

The probes having the nucleotide sequence represented by SEQ ID NOS: 145644, 145645, 145646, 145647, 145648, 145649, 145650, 145651, 145652, 145653, 145654 were selected from the ORFs having the nucleotide sequence represented by SEQ ID NO: 80953.

The probes having the nucleotide sequence represented by SEQ ID NOS: 145655, 145656, 145657, 145658, 145659, 145660, 145661, 145662, 145663, 145664, 145665 were selected from the ORFs having the nucleotide sequence represented by SEQ ID NO: 80954.

The probes having the nucleotide sequence represented by SEQ ID NOS: 145666, 145667, 145668, 145669, 145670, 145671, 145672, 145673, 145674, 145675, 145676 were selected from the ORFs having the nucleotide sequence represented by SEQ ID NO: 80955.

The probes having the nucleotide sequence represented by SEQ ID NOS: 145677, 145678, 145679, 145680, 145681, 145682, 145683, 145684, 145685, 145686, 145687 were selected from the ORFs having the nucleotide sequence represented by SEQ ID NO: 80956.

The probes having the nucleotide sequence represented by SEQ ID NOS: 145688, 145689, 145690, 145691, 145692, 145693, 145694, 145695, 145696, 145697, 145698 were selected from the ORFs having the nucleotide sequence represented by SEQ ID NO: 80957.

The probes having the nucleotide sequence represented by SEQ ID NOS: 145699, 145700, 145701, 145702, 145703, 145704, 145705, 145706, 145707, 145708, 145709 were selected from the ORFs having the nucleotide sequence represented by SEQ ID NO: 80958.

The probes having the nucleotide sequence represented by SEQ ID NOS: 145710, 145711, 145712, 145713, 145714, 145715, 145716, 145717, 145718, 145719, 145720 were selected from the ORFs having the nucleotide sequence represented by SEQ ID NO: 80959.

The probes having the nucleotide sequence represented by SEQ ID NOS: 145721, 145722, 145723, 145724, 145725, 145726, 145727, 145728, 145729, 145730, 145731 were selected from the ORFs having the nucleotide sequence represented by SEQ ID NO: 80960.

The probes having the nucleotide sequence represented by SEQ ID NOS: 145732, 145733, 145734, 145735, 145736, 145737, 145738, 145739, 145740, 145741, 145742 were selected from the ORFs having the nucleotide sequence represented by SEQ ID NO: 80961.

The probes having the nucleotide sequence represented by SEQ ID NOS: 145743, 145744, 145745, 145746, 145747, 145748, 145749, 145750, 145751, 145752, 145753 were selected from the ORFs having the nucleotide sequence represented by SEQ ID NO: 80962.

The probes having the nucleotide sequence represented by SEQ ID NOS: 145754, 145755, 145756, 145757, 145758, 145759, 145760, 145761, 145762, 145763, 145764 were selected from the ORFs having the nucleotide sequence represented by SEQ ID NO: 80963.

The probes having the nucleotide sequence represented by SEQ ID NOS: 145765, 145766, 145767, 145768, 145769, 145770, 145771, 145772, 145773, 145774, 145775 were selected from the ORFs having the nucleotide sequence represented by SEQ ID NO: 80964.

The probes having the nucleotide sequence represented by SEQ ID NOS: 145776, 145777, 145778, 145779, 145780, 145781, 145782, 145783, 145784, 145785, 145786 were selected from the ORFs having the nucleotide sequence represented by SEQ ID NO: 80965.

The probes having the nucleotide sequence represented by SEQ ID NOS: 145787, 145788, 145789, 145790, 145791, 145792, 145793, 145794, 145795, 145796, 145797 were selected from the ORFs having the nucleotide sequence represented by SEQ ID NO: 80966.

The probes having the nucleotide sequence represented by SEQ ID NOS: 145798, 145799, 145800, 145801, 145802, 145803, 145804, 145805, 145806, 145807, 145808 were selected from the ORFs having the nucleotide sequence represented by SEQ ID NO: 80967.

The probes having the nucleotide sequence represented by SEQ ID NOS: 145809, 145810, 145811, 145812, 145813, 145814, 145815, 145816, 145817, 145818, 145819 were selected from the ORFs having the nucleotide sequence represented by SEQ ID NO: 80968.

The probes having the nucleotide sequence represented by SEQ ID NOS: 145820, 145821, 145822, 145823, 145824, 145825, 145826, 145827, 145828, 145829, 145830 were selected from the ORFs having the nucleotide sequence represented by SEQ ID NO: 80969.

The probes having the nucleotide sequence represented by SEQ ID NOS: 145831, 145832, 145833, 145834, 145835, 145836, 145837, 145838, 145839, 145840, 145841 were selected from the ORFs having the,nucleotide sequence represented by SEQ ID NO: 80970.

The probes having the nucleotide sequence represented by SEQ ID NOS: 145842, 145843, 145844, 145845, 145846, 145847, 145848, 145849, 145850 were selected from the ORFs having the nucleotide sequence represented by SEQ ID NO: 80971.

The probes having the nucleotide sequence represented by SEQ ID NOS: 145851, 145852, 145853, 145854, 145855, 145856, 145857, 145858, 145859, 145860 were selected from the ORFs having the nucleotide sequence represented by SEQ ID NO: 80972.

The probes having the nucleotide sequence represented by SEQ ID NOS: 145861, 145862, 145863, 145864, 145865, 145866, 145867, 145868, 145869, 145870, 145871 were selected from the ORFs having the nucleotide sequence represented by SEQ ID NO: 80973.

The probes having the nucleotide sequence represented by SEQ ID NOS: 145872, 145873, 145874, 145875, 145876, 145877, 145878, 145879, 145880, 145881, 145882 were selected from the ORFs having the nucleotide sequence represented by SEQ ID NO: 80974.

The probes having the nucleotide sequence represented by SEQ ID NOS: 145883, 145884, 145885, 145886, 145887, 145888, 145889, 145890, 145891, 145892, 145893 were selected from the ORFs having the nucleotide sequence represented by SEQ ID NO: 80975.

The probes having the nucleotide sequence represented by SEQ ID NOS: 145894, 145895, 145896, 145897, 145898, 145899, 145900, 145901, 145902, 145903, 145904 were selected from the ORFs having the nucleotide sequence represented by SEQ ID NO: 80976.

The probes having the nucleotide sequence represented by SEQ ID NOS: 145905, 145906, 145907, 145908, 145909, 145910, 145911, 145912, 145913, 145914, 145915 were selected from the ORFs having the nucleotide sequence represented by SEQ ID NO: 80977.

The probes having the nucleotide sequence represented by SEQ ID NOS: 145916, 145917, 145918, 145919, 145920, 145921, 145922, 145923, 145924, 145925, 145926 were selected from the ORFs having the nucleotide sequence represented by SEQ ID NO: 80978.

The probes having the nucleotide sequence represented by SEQ ID NOS: 145927, 145928, 145929, 145930, 145931, 145932, 145933, 145934, 145935, 145936, 145937 were selected from the ORFs having the nucleotide sequence represented by SEQ ID NO: 80979.

The probes having the nucleotide sequence represented by SEQ ID NOS: 145938, 145939, 145940, 145941, 145942, 145943, 145944, 145945, 145946, 145947, 145948 were selected from the ORFs having the nucleotide sequence represented by SEQ ID NO: 80980.

The probes having the nucleotide sequence represented by SEQ ID NOS: 145949, 145950, 145951, 145952, 145953, 145954, 145955, 145956, 145957, 145958, 145959 were selected from the ORFs having the nucleotide sequence represented by SEQ ID NO: 80981.

The probes having the nucleotide sequence represented by SEQ ID NOS: 145960, 145961, 145962, 145963, 145964, 145965, 145966, 145967, 145968, 145969, 145970 were selected from the ORFs having the nucleotide sequence represented by SEQ ID NO: 80982.

The probes having the nucleotide sequence represented by SEQ ID NOS: 145971, 145972, 145973, 145974, 145975, 145976, 145977, 145978, 145979, 145980, 145981 were selected from the ORFs having the nucleotide sequence represented by SEQ ID NO: 80983.

The probes having the nucleotide sequence represented by SEQ ID NOS: 145982, 145983, 145984, 145985, 145986, 145987, 145988, 145989, 145990, 145991, 145992 were selected from the ORFs having the nucleotide sequence represented by SEQ ID NO: 80984.

The probes having the nucleotide sequence represented by SEQ ID NOS: 145993, 145994, 145995, 145996, 145997, 145998, 145999, 146000, 146001, 146002, 146003 were selected from the ORFs having the nucleotide sequence represented by SEQ ID NO: 80985.

The probes having the nucleotide sequence represented by SEQ ID NOS: 146004, 146005, 146006, 146007, 146008, 146009, 146010, 146011, 146012, 146013, 146014 were selected from the ORFs having the nucleotide sequence represented by SEQ ID NO: 80986.

The probes having the nucleotide sequence represented by SEQ ID NOS: 146015, 146016, 146017, 146018, 146019, 146020, 146021, 146022, 146023, 146024, 146025, 146026, 146027, 146028, 146029, 146030, 146031, 146032, 146033, 146034, 146035, 146036 were selected from the ORFs having the nucleotide sequence represented by SEQ ID NO: 80987.

The probes having the nucleotide sequence represented by SEQ ID NOS: 146037, 146038, 146039, 146040, 146041, 146042, 146043, 146044, 146045, 146046, 146047 were selected from the ORFs having the nucleotide sequence represented by SEQ ID NO: 80988.

The probes having the nucleotide sequence represented by SEQ ID NOS: 146048, 146049, 146050, 146051, 146052, 146053, 146054, 146055, 146056, 146057, 146058 were selected from the ORFs having the nucleotide sequence represented by SEQ ID NO: 80989.

The probes having the nucleotide sequence represented by SEQ ID NOS: 146059, 146060, 146061, 146062, 146063, 146064, 146065, 146066, 146067, 146068, 146069 were selected from the ORFs having the nucleotide sequence represented by SEQ ID NO: 80990.

The probes having the nucleotide sequence represented by SEQ ID NOS: 146070, 146071, 146072, 146073, 146074, 146075, 146076, 146077, 146078, 146079, 146080 were selected from the ORFs having the nucleotide sequence represented by SEQ ID NO: 80991.

The probes having the nucleotide sequence represented by SEQ ID NOS: 146081, 146082, 146083, 146084, 146085, 146086, 146087, 146088, 146089, 146090, 146091 were selected from the ORFs having the nucleotide sequence represented by SEQ ID NO: 80992.

The probes having the nucleotide sequence represented by SEQ ID NOS: 146092, 146093, 146094, 146095, 146096, 146097, 146098, 146099, 146100, 146101, 146102 were selected from the ORFs having the nucleotide sequence represented by SEQ ID NO: 80993.

The probes having the nucleotide sequence represented by SEQ ID NOS: 146103, 146104, 146105, 146106, 146107, 146108, 146109, 146110, 146111, 146112, 146113 were selected from the ORFs having the nucleotide sequence represented by SEQ ID NO: 80994.

The probes having the nucleotide sequence represented by SEQ ID NOS: 146114, 146115, 146116, 146117, 146118, 146119, 146120, 146121, 146122, 146123, 146124 were selected from the ORFs having the nucleotide sequence represented by SEQ ID NO: 80995.

The probes having the nucleotide sequence represented by SEQ ID NOS: 146125, 146126, 146127, 146128, 146129, 146130, 146131, 146132, 146133, 146134, 146135 were selected from the ORFs having the nucleotide sequence represented by SEQ ID NO: 80996.

The probes having the nucleotide sequence represented by SEQ ID NOS: 146136, 146137, 146138, 146139, 146140, 146141, 146142, 146143, 146144, 146145, 146146 were selected from the ORFs having the nucleotide sequence represented by SEQ ID NO: 80997.

The probes having the nucleotide sequence represented by SEQ ID NOS: 146147, 146148, 146149, 146150, 146151, 146152, 146153, 146154, 146155, 146156, 146157 were selected from the ORFs having the nucleotide sequence represented by SEQ ID NO: 80998.

The probes having the nucleotide sequence represented by SEQ ID NOS: 146158, 146159, 146160, 146161, 146162, 146163, 146164, 146165, 146166, 146167, 146168 were selected from the ORFs having the nucleotide sequence represented by SEQ ID NO: 80999.

The probes having the nucleotide sequence represented by SEQ ID NOS: 146169, 146170, 146171, 146172, 146173, 146174, 146175, 146176, 146177, 146178, 146179 were selected from the ORFs having the nucleotide sequence represented by SEQ ID NO: 81000.

The probes having the nucleotide sequence represented by SEQ ID NOS: 146180, 146181, 146182, 146183, 146184, 146185, 146186, 146187, 146188, 146189, 146190 were selected from the ORFs having the nucleotide sequence represented by SEQ ID NO: 81001.

The probes having the nucleotide sequence represented by SEQ ID NOS: 146191, 146192, 146193, 146194, 146195, 146196, 146197, 146198, 146199, 146200, 146201 were selected from the ORFs having the nucleotide sequence represented by SEQ ID NO: 81002.

The probes having the nucleotide sequence represented by SEQ ID NOS: 146202, 146203, 146204, 146205, 146206, 146207, 146208, 146209, 146210, 146211, 146212 were selected from the ORFs having the nucleotide sequence represented by SEQ ID NO: 81003.

The probes having the nucleotide sequence represented by SEQ ID NOS: 146213, 146214, 146215, 146216, 146217, 146218, 146219, 146220, 146221, 146222, 146223 were selected from the ORFs having the nucleotide sequence represented by SEQ ID NO: 81004.

The probes having the nucleotide sequence represented by SEQ ID NOS: 146224, 146225, 146226, 146227, 146228, 146229, 146230, 146231, 146232, 146233, 146234 were selected from the ORFs having the nucleotide sequence represented by SEQ ID NO: 81005.

The probes having the nucleotide sequence represented by SEQ ID NOS: 146235, 146236, 146237, 146238, 146239, 146240, 146241, 146242, 146243, 146244, 146245 were selected from the ORFs having the nucleotide sequence represented by SEQ ID NO: 81006.

The probes having the nucleotide sequence represented by SEQ ID NOS: 146246, 146247, 146248, 146249, 146250, 146251, 146252, 146253, 146254, 146255, 146256 were selected from the ORFs having the nucleotide sequence represented by SEQ ID NO: 81007.

The probes having the nucleotide sequence represented by SEQ ID NOS: 146257, 146258, 146259, 146260, 146261, 146262, 146263, 146264, 146265, 146266, 146267 were selected from the ORFs having the nucleotide sequence represented by SEQ ID NO: 81008.

The probes having the nucleotide sequence represented by SEQ ID NOS: 146268, 146269, 146270, 146271, 146272, 146273, 146274, 146275, 146276, 146277, 146278 were selected from the ORFs having the nucleotide sequence represented by SEQ ID NO: 81009.

The probes having the nucleotide sequence represented by SEQ ID NOS: 146279, 146280, 146281, 146282, 146283, 146284, 146285, 146286, 146287, 146288, 146289 were selected from the ORFs having the nucleotide sequence represented by SEQ ID NO: 81010.

The probes having the nucleotide sequence represented by SEQ ID NOS: 146290, 146291, 146292, 146293, 146294, 146295, 146296, 146297, 146298, 146299, 146300 were selected from the ORFs having the nucleotide sequence represented by SEQ ID NO: 81011.

The probes having the nucleotide sequence represented by SEQ ID NOS: 146301, 146302, 146303, 146304, 146305, 146306, 146307, 146308, 146309, 146310, 146311 were selected from the ORFs having the nucleotide sequence represented by SEQ ID NO: 81012.

The probes having the nucleotide sequence represented by SEQ ID NOS: 146312, 146313, 146314, 146315, 146316, 146317, 146318, 146319, 146320, 146321, 146322 were selected from the ORFs having the nucleotide sequence represented by SEQ ID NO: 81013.

The probes having the nucleotide sequence represented by SEQ ID NOS: 146323, 146324, 146325, 146326, 146327, 146328, 146329, 146330, 146331, 146332, 146333 were selected from the ORFs having the nucleotide sequence represented by SEQ ID NO: 81014.

The probes having the nucleotide sequence represented by SEQ ID NOS: 146334, 146335, 146336, 146337, 146338, 146339, 146340, 146341, 146342, 146343, 146344 were selected from the ORFs having the nucleotide sequence represented by SEQ ID NO: 81015.

The probes having the nucleotide sequence represented by SEQ ID NOS: 146345, 146346, 146347, 146348, 146349, 146350, 146351, 146352, 146353, 146354, 146355 were selected from the ORFs having the nucleotide sequence represented by SEQ ID NO: 81016.

The probes having the nucleotide sequence represented by SEQ ID NOS: 146356, 146357, 146358, 146359, 146360, 146361, 146362, 146363, 146364, 146365, 146366 were selected from the ORFs having the nucleotide sequence represented by SEQ ID NO: 81017.

The probes having the nucleotide sequence represented by SEQ ID NOS: 146367, 146368, 146369, 146370, 146371, 146372, 146373, 146374, 146375, 146376, 146377 were selected from the ORFs having the nucleotide sequence represented by SEQ ID NO: 81018.

The probes having the nucleotide sequence represented by SEQ ID NOS: 146378, 146379, 146380, 146381, 146382, 146383, 146384, 146385, 146386, 146387, 146388 were selected from the ORFs having the nucleotide sequence represented by SEQ ID NO: 81019.

The probes having the nucleotide sequence represented by SEQ ID NOS: 146389, 146390, 146391, 146392, 146393, 146394, 146395, 146396, 146397, 146398, 146399 were selected from the ORFs having the nucleotide sequence represented by SEQ ID NO: 81020.

The probes having the nucleotide sequence represented by SEQ ID NOS: 146400, 146401, 146402, 146403, 146404, 146405, 146406, 146407, 146408, 146409, 146410 were selected from the ORFs having the nucleotide sequence represented by SEQ ID NO: 81021.

The probes having the nucleotide sequence represented by SEQ ID NOS: 146411, 146412, 146413, 146414, 146415, 146416, 146417, 146418, 146419, 146420, 146421 were selected from the ORFs having the nucleotide sequence represented by SEQ ID NO: 81022.

The probes having the nucleotide sequence represented by SEQ ID NOS: 146422, 146423, 146424, 146425, 146426, 146427, 146428, 146429, 146430, 146431, 146432 were selected from the ORFs having the nucleotide sequence represented by SEQ ID NO: 81023.

The probes having the nucleotide sequence represented by SEQ ID NOS: 146433, 146434, 146435, 146436, 146437, 146438, 146439, 146440, 146441, 146442, 146443 were selected from the ORFs having the nucleotide sequence represented by SEQ ID NO: 81024.

The probes having the nucleotide sequence represented by SEQ ID NOS: 146444, 146445, 146446, 146447, 146448, 146449, 146450, 146451, 146452, 146453, 146454 were selected from the ORFs having the nucleotide sequence represented by SEQ ID NO: 81025.

The probes having the nucleotide sequence represented by SEQ ID NOS: 146455, 146456, 146457, 146458, 146459, 146460, 146461, 146462, 146463, 146464, 146465 were selected from the ORFs having the nucleotide sequence represented by SEQ ID NO: 81026.

The probes having the nucleotide sequence represented by SEQ ID NOS: 146466, 146467, 146468, 146469, 146470, 146471, 146472, 146473, 146474, 146475, 146476 were selected from the ORFs having the nucleotide sequence represented by SEQ ID NO: 81027.

The probes having the nucleotide sequence represented by SEQ ID NOS: 146477, 146478, 146479, 146480, 146481, 146482, 146483, 146484, 146485, 146486, 146487 were selected from the ORFs having the nucleotide sequence represented by SEQ ID NO: 81028.

The probes having the nucleotide sequence represented by SEQ ID NOS: 146488, 146489, 146490, 146491, 146492, 146493, 146494, 146495, 146496, 146497, 146498 were selected from the ORFs having the nucleotide sequence represented by SEQ ID NO: 81029.

The probes having the,nucleotide sequence represented by SEQ ID NOS: 146499, 146500, 146501, 146502, 146503, 146504, 146505, 146506, 146507, 146508, 146509 were selected from the ORFs having the nucleotide sequence represented by SEQ ID NO: 81030.

The probes having the nucleotide sequence represented by SEQ ID NOS: 146510, 146511, 146512, 146513, 146514, 146515, 146516, 146517, 146518, 146519, 146520 were selected from the ORFs having the nucleotide sequence represented by SEQ ID NO: 81031.

The probes having the nucleotide sequence represented by SEQ ID NOS: 146521, 146522, 146523, 146524, 146525, 146526, 146527, 146528, 146529, 146530, 146531 were selected from the ORFs having the nucleotide sequence represented by SEQ ID NO: 81032.

The probes having the nucleotide sequence represented by SEQ ID NOS: 146532, 146533, 146534, 146535, 146536, 146537, 146538, 146539, 146540, 146541, 146542 were selected from the ORFs having the nucleotide sequence represented by SEQ ID NO: 81033.

The probes having the nucleotide sequence represented by SEQ ID NOS: 146543, 146544, 146545, 146546, 146547, 146548, 146549, 146550, 146551, 146552, 146553 were selected from the ORFs having the nucleotide sequence represented by SEQ ID NO: 81034.

The probes having the nucleotide sequence represented by SEQ ID NOS: 146554, 146555, 146556, 146557, 146558, 146559, 146560, 146561, 146562, 146563, 146564 were selected from the ORFs having the nucleotide sequence represented by SEQ ID NO: 81035.

The probes having the nucleotide sequence represented by SEQ ID NOS: 146565, 146566, 146567, 146568, 146569, 146570, 146571, 146572, 146573, 146574, 146575 were selected from the ORFs having the nucleotide sequence represented by SEQ ID NO: 81036.

The probes having the nucleotide sequence represented by SEQ ID NOS: 146576, 146577, 146578, 146579, 146580, 146581, 146582, 146583, 146584, 146585, 146586 were selected from the ORFs having the nucleotide sequence represented by SEQ ID NO: 81037.

The probes having the nucleotide sequence represented by SEQ ID NOS: 146587, 146588, 146589, 146590, 146591, 146592, 146593, 146594, 146595, 146596, 146597 were selected from the ORFs having the nucleotide sequence represented by SEQ ID NO: 81038.

The probes having the nucleotide sequence represented by SEQ ID NOS: 146598, 146599, 146600, 146601, 146602, 146603, 146604, 146605, 146606, 146607, 146608 were selected from the ORFs having the nucleotide sequence represented by SEQ ID NO: 81039.

The probes having the nucleotide sequence represented by SEQ ID NOS: 146609, 146610, 146611, 146612, 146613, 146614, 146615, 146616, 146617, 146618, 146619 were selected from the ORFs having the nucleotide sequence represented by SEQ ID NO: 81040.

The probes having the nucleotide sequence represented by SEQ ID NOS: 146620, 146621, 146622, 146623, 146624, 146625, 146626, 146627, 146628, 146629, 146630 were selected from the ORFs having the nucleotide sequence represented by SEQ ID NO: 81041.

The probes having the nucleotide sequence represented by SEQ ID NOS: 146631, 146632, 146633, 146634, 146635, 146636, 146637, 146638, 146639, 146640, 146641 were selected from the ORFs having the nucleotide sequence represented by SEQ ID NO: 81042.

The probes having the nucleotide sequence represented by SEQ ID NOS: 146642, 146643, 146644, 146645, 146646, 146647, 146648, 146649, 146650, 146651, 146652 were selected from the ORFs having the nucleotide sequence represented by SEQ ID NO: 81043.

The probes having the nucleotide sequence represented by SEQ ID NOS: 146653, 146654, 146655, 146656, 146657, 146658, 146659, 146660, 146661, 146662, 146663 were selected from the ORFs having the nucleotide sequence represented by SEQ ID NO: 81044.

The probes having the nucleotide sequence represented by SEQ ID NOS: 146664, 146665, 146666, 146667, 146668, 146669, 146670, 146671, 146672, 146673, 146674 were selected from the ORFs having the nucleotide sequence represented by SEQ ID NO: 81045.

The probes having the nucleotide sequence represented by SEQ ID NOS: 146675, 146676, 146677, 146678, 146679, 146680, 146681, 146682, 146683, 146684, 146685 were selected from the ORFs having the nucleotide sequence represented by SEQ ID NO: 81046.

The probes having the nucleotide sequence represented by SEQ ID NOS: 146686, 146687, 146688, 146689, 146690, 146691, 146692, 146693, 146694, 146695, 146696 were selected from the ORFs having the nucleotide sequence represented by SEQ ID NO: 81047.

The probes having the nucleotide sequence represented by SEQ ID NOS: 146697, 146698, 146699, 146700, 146701, 146702, 146703, 146704, 146705, 146706, 146707 were selected from the ORFs having the nucleotide sequence represented by SEQ ID NO: 81048.

The probes having the nucleotide sequence represented by SEQ ID NOS: 146708, 146709, 146710, 146711, 146712, 146713, 146714, 146715, 146716, 146717, 146718 were selected from the ORFs having the nucleotide sequence represented by SEQ ID NO: 81049.

The probes having the nucleotide sequence represented by SEQ ID NOS: 146719, 146720, 146721, 146722, 146723, 146724, 146725, 146726, 146727, 146728, 146729 were selected from the ORFs having the nucleotide sequence represented by SEQ ID NO: 81050.

The probes having the nucleotide sequence represented by SEQ ID NOS: 146730, 146731, 146732, 146733, 146734, 146735, 146736, 146737, 146738, 146739, 146740 were selected from the ORFs having the nucleotide sequence represented by SEQ ID NO: 81051.

The probes having the nucleotide sequence represented by SEQ ID NOS: 146741, 146742, 146743, 146744, 146745, 146746, 146747, 146748, 146749, 146750, 146751 were selected from the ORFs having the nucleotide sequence represented by SEQ ID NO: 81052.

The probes having the nucleotide sequence represented by SEQ ID NOS: 146752, 146753, 146754, 146755, 146756, 146757, 146758, 146759, 146760, 146761, 146762 were selected from the ORFs having the nucleotide sequence represented by SEQ ID NO: 81053.

The probes having the nucleotide sequence represented by SEQ ID NOS: 146763, 146764, 146765, 146766, 146767, 146768, 146769, 146770, 146771, 146772, 146773 were selected from the ORFs having the nucleotide sequence represented by SEQ ID NO: 81054.

The probes having the nucleotide sequence represented by SEQ ID NOS: 146774, 146775, 146776, 146777, 146778, 146779, 146780, 146781, 146782, 146783, 146784 were selected from the ORFs having the nucleotide sequence represented by SEQ ID NO: 81055.

The probes having the nucleotide sequence represented by SEQ ID NOS: 146785, 146786, 146787, 146788, 146789, 146790, 146791, 146792, 146793, 146794, 146795 were selected from the ORFs having the nucleotide sequence represented by SEQ ID NO: 81056.

The probes having the nucleotide sequence represented by SEQ ID NOS: 146796, 146797, 146798, 146799, 146800, 146801, 146802, 146803, 146804, 146805, 146806 were selected from the ORFs having the nucleotide sequence represented by SEQ ID NO: 81057.

The probes having the nucleotide sequence represented by SEQ ID NOS: 146807, 146808, 146809, 146810, 146811, 146812, 146813, 146814, 146815, 146816, 146817 were selected from the ORFs having the nucleotide sequence represented by SEQ ID NO: 81058.

The probes having the nucleotide sequence represented by SEQ ID NOS: 146818, 146819, 146820, 146821, 146822, 146823, 146824, 146825, 146826, 146827, 146828 were selected from the ORFs having the nucleotide sequence represented by SEQ ID NO: 81059.

The probes having the nucleotide sequence represented by SEQ ID NOS: 146829, 146830, 146831, 146832, 146833, 146834, 146835, 146836, 146837, 146838, 146839 were selected from the ORFs having the nucleotide sequence represented by SEQ ID NO: 81060.

The probes having the nucleotide sequence represented by SEQ ID NOS: 146840, 146841, 146842, 146843, 146844, 146845, 146846, 146847, 146848, 146849, 146850 were selected from the ORFs having the nucleotide sequence represented by SEQ ID NO: 81061.

The probes having the nucleotide sequence represented by SEQ ID NOS: 146851, 146852, 146853, 146854, 146855, 146856, 146857, 146858, 146859, 146860, 146861 were selected from the ORFs having the nucleotide sequence represented by SEQ ID NO: 81062.

The probes having the nucleotide sequence represented by SEQ ID NOS: 146862, 146863, 146864, 146865, 146866, 146867, 146868, 146869, 146870, 146871, 146872 were selected from the ORFs having the nucleotide sequence represented by SEQ ID NO: 81063.

The probes having the nucleotide sequence represented by SEQ ID NOS: 146873, 146874, 146875, 146876, 146877, 146878, 146879, 146880, 146881, 146882, 146883 were selected from the ORFs having the nucleotide sequence represented by SEQ ID NO: 81064.

The probes having the nucleotide sequence represented by SEQ ID NOS: 146884, 146885, 146886, 146887, 146888, 146889, 146890, 146891, 146892, 146893, 146894 were selected from the ORFs having the nucleotide sequence represented by SEQ ID NO: 81065.

The probes having the nucleotide sequence represented by SEQ ID NOS: 146895, 146896, 146897, 146898, 146899, 146900, 146901, 146902, 146903, 146904, 146905 were selected from the ORFs having the nucleotide sequence represented by SEQ ID NO: 81066.

The probes having the nucleotide sequence represented by SEQ ID NOS: 146906, 146907, 146908, 146909, 146910, 146911, 146912, 146913, 146914, 146915, 146916 were selected from the ORFs having the nucleotide sequence represented by SEQ ID NO: 81067.

The probes having the nucleotide sequence represented by SEQ ID NOS: 146917, 146918, 146919, 146920, 146921, 146922, 146923, 146924, 146925, 146926, 146927 were selected from the ORFs having the nucleotide sequence represented by SEQ ID NO: 81068.

The probes having the nucleotide sequence represented by SEQ ID NOS: 146928, 146929, 146930, 146931, 146932, 146933, 146934, 146935, 146936, 146937, 146938 were selected from the ORFs having the nucleotide sequence represented by SEQ ID NO: 81069.

The probes having the nucleotide sequence represented by SEQ ID NOS: 146939, 146940, 146941, 146942, 146943, 146944, 146945, 146946, 146947, 146948, 146949 were selected from the ORFs having the nucleotide sequence represented by SEQ ID NO: 81070.

The probes having the nucleotide sequence represented by SEQ ID NOS: 146950, 146951, 146952, 146953, 146954, 146955, 146956, 146957, 146958, 146959, 146960 were selected from the ORFs having the nucleotide sequence represented by SEQ ID NO: 81071.

The probes having the nucleotide sequence represented by SEQ ID NOS: 146961, 146962, 146963, 146964, 146965, 146966, 146967, 146968, 146969, 146970, 146971 were selected from the ORFs having the nucleotide sequence represented by SEQ ID NO: 81072.

The probes having the nucleotide sequence represented by SEQ ID NOS: 146972, 146973, 146974, 146975, 146976, 146977, 146978, 146979, 146980, 146981, 146982 were selected from the ORFs having the nucleotide sequence represented by SEQ ID NO: 81073.

The probes having the nucleotide sequence represented by SEQ ID NOS: 146983, 146984, 146985, 146986, 146987, 146988, 146989, 146990, 146991, 146992, 146993 were selected from the ORFs having the nucleotide sequence represented by SEQ ID NO: 81074.

The probes having the nucleotide sequence represented by SEQ ID NOS: 146994, 146995, 146996, 146997, 146998, 146999, 147000, 147001, 147002, 147003, 147004 were selected from the ORFs having the nucleotide sequence represented by SEQ ID NO: 81075.

The probes having the nucleotide sequence represented by SEQ ID NOS: 147005, 147006, 147007, 147008, 147009, 147010, 147011, 147012, 147013, 147014, 147015 were selected from the ORFs having the nucleotide sequence represented by SEQ ID NO: 81076.

The probes having the nucleotide sequence represented by SEQ ID NOS: 147016, 147017, 147018, 147019, 147020, 147021, 147022, 147023, 147024, 147025, 147026 were selected from the ORFs having the nucleotide sequence represented by SEQ ID NO: 81077.

The probes having the nucleotide sequence represented by SEQ ID NOS: 147027, 147028, 147029, 147030, 147031, 147032, 147033, 147034, 147035, 147036, 147037 were selected from the ORFs having the nucleotide sequence represented by SEQ ID NO: 81078.

The probes having the nucleotide sequence represented by SEQ ID NOS: 147038, 147039, 147040, 147041, 147042, 147043, 147044, 147045, 147046, 147047, 147048 were selected from the ORFs having the nucleotide sequence represented by SEQ ID NO: 81079.

The probes having the nucleotide sequence represented by SEQ ID NOS: 147049, 147050, 147051, 147052, 147053, 147054, 147055, 147056, 147057, 147058, 147059 were selected from the ORFs having the nucleotide sequence represented by SEQ ID NO: 81080.

The probes having the nucleotide sequence represented by SEQ ID NOS: 147060, 147061, 147062, 147063, 147064, 147065, 147066, 147067, 147068, 147069, 147070 were selected from the ORFs having the nucleotide sequence represented by SEQ ID NO: 81081.

The probes having the nucleotide sequence represented by SEQ ID NOS: 147071, 147072, 147073, 147074, 147075, 147076, 147077, 147078, 147079, 147080, 147081 were selected from the ORFs having the nucleotide sequence represented by SEQ ID NO: 81082.

The probes having the nucleotide sequence represented by SEQ ID NOS: 147082, 147083, 147084, 147085, 147086, 147087, 147088, 147089, 147090, 147091, 147092 were selected from the ORFs having the nucleotide sequence represented by SEQ ID NO: 81083.

The probes having the nucleotide sequence represented by SEQ ID NOS: 147093, 147094, 147095, 147096, 147097, 147098, 147099, 147100, 147101, 147102, 147103 were selected from the ORFs having the nucleotide sequence represented by SEQ ID NO: 81084.

The probes having the nucleotide sequence represented by SEQ ID NOS: 147104, 147105, 147106, 147107, 147108, 147109, 147110, 147111, 147112, 147113, 147114 were selected from the ORFs having the nucleotide sequence represented by SEQ ID NO: 81085.

The probes having the nucleotide sequence represented by SEQ ID NOS: 147115, 147116, 147117, 147118, 147119, 147120, 147121, 147122, 147123, 147124, 147125 were selected from the ORFs having the nucleotide sequence represented by SEQ ID NO: 81086.

The probes having the nucleotide sequence represented by SEQ ID NOS: 147126, 147127, 147128, 147129, 147130, 147131, 147132, 147133, 147134, 147135, 147136 were selected from the ORFs having the nucleotide sequence represented by SEQ ID NO: 81087.

The probes having the nucleotide sequence represented by SEQ ID NOS: 147137, 147138, 147139, 147140, 147141, 147142, 147143, 147144, 147145, 147146, 147147 were selected from the ORFs having the nucleotide sequence represented by SEQ ID NO: 81088.

The probes having the nucleotide sequence represented by SEQ ID NOS: 147148, 147149, 147150, 147151, 147152, 147153, 147154, 147155, 147156, 147157, 147158 were selected from the ORFs having the nucleotide sequence represented by SEQ ID NO: 81089.

The probes having the nucleotide sequence represented by SEQ ID NOS: 147159, 147160, 147161, 147162, 147163, 147164, 47165, 147166, 147167, 147168, 147169 were selected from the ORFs having the nucleotide sequence represented by SEQ ID NO: 81090.

The probes having the nucleotide sequence represented by SEQ ID NOS: 147170, 147171, 147172, 147173, 147174, 147175, 147176, 147177, 147178, 147179, 147180 were selected from the ORFs having the nucleotide sequence represented by SEQ ID NO: 81091.

The probes having the nucleotide sequence represented by SEQ ID NOS: 147181, 147182, 147183, 147184, 147185, 147186, 147187, 147188, 147189, 147190, 147191 were selected from the ORFs having the nucleotide sequence represented by SEQ ID NO: 81092.

The probes having the nucleotide sequence represented by SEQ ID NOS: 147192, 147193, 147194, 147195, 147196, 147197, 147198, 147199, 147200, 147201, 147202 were selected from the ORFs having the nucleotide sequence represented by SEQ ID NO: 81093.

The probes having the nucleotide sequence represented by SEQ ID NOS: 147203, 147204, 147205, 147206, 147207, 147208, 147209, 147210, 147211, 147212, 147213 were selected from the ORFs having the nucleotide sequence represented by SEQ ID NO: 81094.

The probes having the nucleotide sequence represented by SEQ ID NOS: 147214, 147215, 147216, 147217, 147218, 147219, 147220, 147221, 147222, 147223, 147224 were selected from the ORFs having the nucleotide sequence represented by SEQ ID NO: 81095.

The probes having the nucleotide sequence represented by SEQ ID NOS: 147225, 147226, 147227, 147228, 147229, 147230, 147231, 147232, 147233, 147234, 147235 were selected from the ORFs having the nucleotide sequence represented by SEQ ID NO: 81096.

The probes having the nucleotide sequence represented by SEQ ID NOS: 147236, 147237, 147238, 147239, 147240, 147241, 147242, 147243, 147244, 147245, 147246 were selected from the ORFs having the nucleotide sequence represented by SEQ ID NO: 81097.

The probes having the nucleotide sequence represented by SEQ ID NOS: 147247, 147248, 147249, 147250, 147251, 147252, 147253, 147254, 147255, 147256, 147257 were selected from the ORFs having the nucleotide sequence represented by SEQ ID NO: 81098.

The probes having the nucleotide sequence represented by SEQ ID NOS: 147258, 147259, 147260, 147261, 147262, 147263, 147264, 147265, 147266, 147267, 147268, 147269, 147270, 147271, 147272, 147273, 147274, 147275, 147276, 147277, 147278, 147279 were selected from the ORFs having the nucleotide sequence represented by SEQ ID NO: 81099.

The probes having the nucleotide sequence represented by SEQ ID NOS: 147280, 147281, 147282, 147283, 147284, 147285, 147286, 147287, 147288, 147289, 147290 were selected from the ORFs having the nucleotide sequence represented by SEQ ID NO: 81100.

The probes having the nucleotide sequence represented by SEQ ID NOS: 147291, 147292, 147293, 147294, 147295, 147296, 147297, 147298, 147299, 147300, 147301 were selected from the ORFs having the nucleotide sequence represented by SEQ ID NO: 81101.

The probes having the nucleotide sequence represented by SEQ ID NOS: 147302, 147303, 147304, 147305, 147306, 147307, 147308, 147309, 147310, 147311, 147312 were selected from the ORFs having the nucleotide sequence represented by SEQ ID NO: 81102.

The probes having the nucleotide sequence represented by SEQ ID NOS: 147313, 147314, 147315, 147316, 147317, 147318, 147319, 147320, 147321, 147322, 147323 were selected from the ORFs having the nucleotide sequence represented by SEQ ID NO: 81103.

The probes having the nucleotide sequence represented by SEQ ID NOS: 147324, 147325, 147326, 147327, 147328, 147329, 147330, 147331, 147332, 147333, 147334 were selected from the ORFs having the nucleotide sequence represented by SEQ ID NO: 81104.

The probes having the nucleotide sequence represented by SEQ ID NOS: 147335, 147336, 147337, 147338, 147339, 147340, 147341, 147342, 147343, 147344, 147345 were selected from the ORFs having the nucleotide sequence represented by SEQ ID NO: 81105.

The probes having the nucleotide sequence represented by SEQ ID NOS: 147346, 147347, 147348, 147349, 147350, 147351, 147352, 147353, 147354, 147355, 147356 were selected from the ORFs having the nucleotide sequence represented by SEQ ID NO: 81106.

The probes having the nucleotide sequence represented by SEQ ID NOS: 147357, 147358, 147359, 147360, 147361, 147362, 147363, 147364, 147365, 147366, 147367 were selected from the ORFs having the nucleotide sequence represented by SEQ ID NO: 81107.

The probes having the nucleotide sequence represented by SEQ ID NOS: 147368, 147369, 147370, 147371, 147372, 147373, 147374, 147375, 147376, 147377, 147378 were selected from the ORFs having the nucleotide sequence represented by SEQ ID NO: 81108.

The probes having the nucleotide sequence represented by SEQ ID NOS: 147379, 147380, 147381, 147382, 147383, 147384, 147385, 147386, 147387, 147388, 147389 were selected from the ORFs having the nucleotide sequence represented by SEQ ID NO: 81109.

The probes having the nucleotide sequence represented by SEQ ID NOS: 147390, 147391, 147392, 147393, 147394, 147395, 147396, 147397, 147398, 147399, 147400 were selected from the ORFs having the nucleotide sequence represented by SEQ ID NO: 81110.

The probes having the nucleotide sequence represented by SEQ ID NOS: 147401, 147402, 147403, 147404, 147405, 147406, 147407, 147408, 147409, 147410, 147411 were selected from the ORFs having the nucleotide sequence represented by SEQ ID NO: 81111.

The probes having the nucleotide sequence represented by SEQ ID NOS: 147412, 147413, 147414, 147415, 147416, 147417, 147418, 147419, 147420, 147421, 147422 were selected from the ORFs having the nucleotide sequence represented by SEQ ID NO: 81112.

The probes having the nucleotide sequence represented by SEQ ID NOS: 147423, 147424, 147425, 147426, 147427, 147428, 147429, 147430, 147431, 147432, 147433 were selected from the ORFs having the nucleotide sequence represented by SEQ ID NO: 81113.

The probes having the nucleotide sequence represented by SEQ ID NOS: 147434, 147435, 147436, 147437, 147438, 147439, 147440, 147441, 147442, 147443, 147444 were selected from the ORFs having the nucleotide sequence represented by SEQ ID NO: 81114.

The probes having the nucleotide sequence represented by SEQ ID NOS: 147445, 147446, 147447, 147448, 147449, 147450, 147451, 147452, 147453, 147454, 147455 were selected from the ORFs having the nucleotide sequence represented by SEQ ID NO: 81115.

The probes having the nucleotide sequence represented by SEQ ID NOS: 147456, 147457, 147458, 147459, 147460, 147461, 147462, 147463, 147464, 147465, 147466 were selected from the ORFs having the nucleotide sequence represented by SEQ ID NO: 81116.

The probes having the nucleotide sequence represented by SEQ ID NOS: 147467, 147468, 147469, 147470, 147471, 147472, 147473, 147474, 147475, 147476, 147477 were selected from the ORFs having the nucleotide sequence represented by SEQ ID NO: 81117.

The probes having the nucleotide sequence represented by SEQ ID NOS: 147478, 147479, 147480, 147481, 147482, 147483, 147484, 147485, 147486, 147487, 147488 were selected from the ORFs having the nucleotide sequence represented by SEQ ID NO: 81118.

The probes having the nucleotide sequence represented by SEQ ID NOS: 147489, 147490, 147491, 147492, 147493, 147494, 147495, 147496, 147497, 147498, 147499 were selected from the ORFs having the nucleotide sequence represented by SEQ ID NO: 81119.

The probes having the nucleotide sequence represented by SEQ ID NOS: 147500, 147501, 147502, 147503, 147504, 147505, 147506, 147507, 147508, 147509, 147510 were selected from the ORFs having the nucleotide sequence represented by SEQ ID NO: 81120.

The probes having the nucleotide sequence represented by SEQ ID NOS: 147511, 147512, 147513, 147514, 147515, 147516, 147517, 147518, 147519, 147520, 147521 were selected from the ORFs having the nucleotide sequence represented by SEQ ID NO: 81121.

The probes having the nucleotide sequence represented by SEQ ID NOS: 147522, 147523, 147524, 147525, 147526, 147527, 147528, 147529, 147530, 147531, 147532 were selected from the ORFs having the nucleotide sequence represented by SEQ ID NO: 81122.

The probes having the nucleotide sequence represented by SEQ ID NOS: 147533, 147534, 147535, 147536, 147537, 147538, 147539, 147540, 147541, 147542, 147543 were selected from the ORFs having the nucleotide sequence represented by SEQ ID NO: 81123.

The probes having the nucleotide sequence represented by SEQ ID NOS: 147544, 147545, 147546, 147547, 147548, 147549, 147550, 147551, 147552, 147553, 147554 were selected from the ORFs having the nucleotide sequence represented by SEQ ID NO: 81124.

The probes having the nucleotide sequence represented by SEQ ID NOS: 147555, 147556, 147557, 147558, 147559, 147560, 147561, 147562, 147563, 147564, 147565 were selected from the ORFs having the nucleotide sequence represented by SEQ ID NO: 81125.

The probes having the nucleotide sequence represented by SEQ ID NOS: 147566, 147567, 147568, 147569, 147570, 147571, 147572, 147573, 147574, 147575, 147576 were selected from the ORFs having the nucleotide sequence represented by SEQ ID NO: 81126.

The probes having the nucleotide sequence represented by SEQ ID NOS: 147577, 147578, 147579, 147580, 147581, 147582, 147583, 147584, 147585, 147586, 147587 were selected from the ORFs having the nucleotide sequence represented by SEQ ID NO: 81127.

The probes having the nucleotide sequence represented by SEQ ID NOS: 147588, 147589, 147590, 147591, 147592, 147593, 147594, 147595, 147596, 147597, 147598 were selected from the ORFs having the nucleotide sequence represented by SEQ ID NO: 81128.

The probes having the nucleotide sequence represented by SEQ ID NOS: 147599, 147600, 147601, 147602, 147603, 147604, 147605, 147606, 147607, 147608, 147609 were selected from the ORFs having the nucleotide sequence represented by SEQ ID NO: 81129.

The probes having the nucleotide sequence represented by SEQ ID NOS: 147610, 147611, 147612, 147613, 147614, 147615, 147616, 147617, 147618, 147619, 147620 were selected from the ORFs having the nucleotide sequence represented by SEQ ID NO: 81130.

The probes having the nucleotide sequence represented by SEQ ID NOS: 147621, 147622, 147623, 147624, 147625, 147626, 147627, 147628, 147629, 147630, 147631 were selected from the ORFs having the nucleotide sequence represented by SEQ ID NO: 81131.

The probes having the nucleotide sequence represented by SEQ ID NOS: 147632, 147633, 147634, 147635, 147636, 147637, 147638, 147639, 147640, 147641, 147642 were selected from the ORFs having the nucleotide sequence represented by SEQ ID NO: 81132.

The probes having the nucleotide sequence represented by SEQ ID NOS: 147643, 147644, 147645, 147646, 147647, 147648, 147649, 147650, 147651, 147652, 147653, 147654, 147655, 147656, 147657, 147658, 147659, 147660, 147661, 147662, 147663, 147664 were selected from the ORFs having the nucleotide sequence represented by SEQ ID NO: 81133.

The probes having the nucleotide sequence represented by SEQ ID NOS: 147665, 147666, 147667, 147668, 147669, 147670, 147671, 147672, 147673, 147674, 147675 were selected from the ORFs having the nucleotide sequence represented by SEQ ID NO: 81134.

The probes having the nucleotide sequence represented by SEQ ID NOS: 147676, 147677, 147678, 147679, 147680, 147681, 147682, 147683, 147684, 147685, 147686 were selected from the ORFs having the nucleotide sequence represented by SEQ ID NO: 81135.

The probes having the nucleotide sequence represented by SEQ ID NOS: 147687, 147688, 147689, 147690, 147691, 147692, 147693, 147694, 147695, 147696, 147697 were selected from the ORFs having the nucleotide sequence represented by SEQ ID NO: 81136.

The probes having the nucleotide sequence represented by SEQ ID NOS: 147698, 147699, 147700, 147701, 147702, 147703, 147704, 147705, 147706, 147707, 147708 were selected from the ORFs having the nucleotide sequence represented by SEQ ID NO: 81137.

The probes having the nucleotide sequence represented by SEQ ID NOS: 147709, 147710, 147711, 147712, 147713, 147714, 147715, 147716, 147717, 147718, 147719 were selected from the ORFs having the nucleotide sequence represented by SEQ ID NO: 81138.

The probes having the nucleotide sequence represented by SEQ ID NOS: 147720, 147721, 147722, 147723, 147724, 147725, 147726, 147727, 147728, 147729, 147730 were selected from the ORFs having the nucleotide sequence represented by SEQ ID NO: 81139.

The probes having the nucleotide sequence represented by SEQ ID NOS: 147731, 147732, 147733, 147734, 147735, 147736, 147737, 147738, 147739, 147740, 147741 were selected from the ORFs having the nucleotide sequence represented by SEQ ID NO: 81140.

The probes having the nucleotide sequence represented by SEQ ID NOS: 147742, 147743, 147744, 147745, 147746, 147747, 147748, 147749, 147750, 147751, 147752 were selected from the ORFs having the nucleotide sequence represented by SEQ ID NO: 81141.

The probes having the nucleotide sequence represented by SEQ ID NOS: 147753, 147754, 147755, 147756, 147757, 147758, 147759, 147760, 147761, 147762, 147763 were selected from the ORFs having the nucleotide sequence represented by SEQ ID NO: 81142.

The probes having the nucleotide sequence represented by SEQ ID NOS: 147764, 147765, 147766, 147767, 147768, 147769, 147770, 147771, 147772, 147773, 147774 were selected from the ORFs having the nucleotide sequence represented by SEQ ID NO: 81143.

The probes having the nucleotide sequence represented by SEQ ID NOS: 147775, 147776, 147777, 147778, 147779, 147780, 147781, 147782, 147783, 147784, 147785 were selected from the ORFs having the nucleotide sequence represented by SEQ ID NO: 81144.

The probes having the nucleotide sequence represented by SEQ ID NOS: 147786, 147787, 147788, 147789, 147790, 147791, 147792, 147793, 147794, 147795, 147796 were selected from the ORFs having the nucleotide sequence represented by SEQ ID NO: 81145.

The probes having the nucleotide sequence represented by SEQ ID NOS: 147797, 147798, 147799, 147800, 147801, 147802, 147803, 147804, 147805, 147806, 147807 were selected from the ORFs having the nucleotide sequence represented by SEQ ID NO: 81146.

The probes having the nucleotide sequence represented by SEQ ID NOS: 147808, 147809, 147810, 147811, 147812, 147813, 147814, 147815, 147816, 147817, 147818 were selected from the ORFs having the nucleotide sequence represented by SEQ ID NO: 81147.

The probes having the nucleotide sequence represented by SEQ ID NOS: 147819, 147820, 147821, 147822, 147823, 147824, 147825, 147826, 147827, 147828, 147829 were selected from the ORFs having the nucleotide sequence represented by SEQ ID NO: 81148.

The probes having the nucleotide sequence represented by SEQ ID NOS: 147830, 147831, 147832, 147833, 147834, 147835, 147836, 147837, 147838, 147839, 147840 were selected from the ORFs having the nucleotide sequence represented by SEQ ID NO: 81149.

The probes having the nucleotide sequence represented by SEQ ID NOS: 147841, 147842, 147843, 147844, 147845, 147846, 147847, 147848, 147849, 147850, 147851 were selected from the ORFs having the nucleotide sequence represented by SEQ ID NO: 81150.

The probes having the nucleotide sequence represented by SEQ ID NOS: 147852, 147853, 147854, 147855, 147856, 147857, 147858, 147859, 147860, 147861, 147862 were selected from the ORFs having the nucleotide sequence represented by SEQ ID NO: 81151.

The probes having the nucleotide sequence represented by SEQ ID NOS: 147863, 147864, 147865, 147866, 147867, 147868, 147869, 147870, 147871, 147872, 147873 were selected from the ORFs having the nucleotide sequence represented by SEQ ID NO: 81152.

The probes having the nucleotide sequence represented by SEQ ID NOS: 147874, 147875, 147876, 147877, 147878, 147879, 147880, 147881, 147882, 147883, 147884 were selected from the ORFs having the nucleotide sequence represented by SEQ ID NO: 81153.

The probes having the nucleotide sequence represented by SEQ ID NOS: 147885, 147886, 147887, 147888, 147889, 147890, 147891, 147892, 147893, 147894, 147895 were selected from the ORFs having the nucleotide sequence represented by SEQ ID NO: 81154.

The probes having the nucleotide sequence represented by SEQ ID NOS: 147896, 147897, 147898, 147899, 147900, 147901, 147902, 147903, 147904, 147905, 147906 were selected from the ORFs having the nucleotide sequence represented by SEQ ID NO: 81155.

The probes having the nucleotide sequence represented by SEQ ID NOS: 147907, 147908, 147909, 147910, 147911, 147912, 147913, 147914, 147915, 147916, 147917 were selected from the ORFs having the nucleotide sequence represented by SEQ ID NO: 81156.

The probes having the nucleotide sequence represented by SEQ ID NOS: 147918, 147919, 147920, 147921, 147922, 147923, 147924, 147925, 147926, 147927, 147928 were selected from the ORFs having the nucleotide sequence represented by SEQ ID NO: 81157.

The probes having the nucleotide sequence represented by SEQ ID NOS: 147929, 147930, 147931, 147932, 147933, 147934, 147935, 147936, 147937, 147938, 147939 were selected from the ORFs having the nucleotide sequence represented by SEQ ID NO: 81158.

The probes having the nucleotide sequence represented by SEQ ID NOS: 147940, 147941, 147942, 147943, 147944, 147945, 147946, 147947, 147948, 147949, 147950 were selected from the ORFs having the nucleotide sequence represented by SEQ ID NO: 81159.

The probes having the nucleotide sequence represented by SEQ ID NOS: 147951, 147952, 147953, 147954, 147955, 147956, 147957, 147958, 147959, 147960, 147961 were selected from the ORFs having the nucleotide sequence represented by SEQ ID NO: 81160.

The probes having the nucleotide sequence represented by SEQ ID NOS: 147962, 147963, 147964, 147965, 147966, 147967, 147968, 147969, 147970, 147971, 147972 were selected from the ORFs having the nucleotide sequence represented by SEQ ID NO: 81161.

The probes having the nucleotide sequence represented by SEQ ID NOS: 147973, 147974, 147975, 147976, 147977, 147978, 147979, 147980, 147981, 147982, 147983 were selected from the ORFs having the nucleotide sequence represented by SEQ ID NO: 81162.

The probes having the nucleotide sequence represented by SEQ ID NOS: 147984, 147985, 147986, 147987, 147988, 147989, 147990, 147991, 147992, 147993, 147994 were selected from the ORFs having the nucleotide sequence represented by SEQ ID NO: 81163.

The probes having the nucleotide sequence represented by SEQ ID NOS: 147995, 147996, 147997, 147998, 147999, 148000, 148001, 148002, 148003, 148004, 148005 were selected from the ORFs having the nucleotide sequence represented by SEQ ID NO: 81164.

The probes having the nucleotide sequence represented by SEQ ID NOS: 148006, 148007, 148008, 148009, 148010, 148011, 148012, 148013, 148014, 148015, 148016 were selected from the ORFs having the nucleotide sequence represented by SEQ ID NO: 81165.

The probes having the nucleotide sequence represented by SEQ ID NOS: 148017, 148018, 148019, 148020, 148021, 148022, 148023, 148024, 148025, 148026, 148027 were selected from the ORFs having the nucleotide sequence represented by SEQ ID NO: 81166.

The probes having the nucleotide sequence represented by SEQ ID NOS: 148028, 148029, 148030, 148031, 148032, 148033, 148034, 148035, 148036, 148037, 148038 were selected from the ORFs having the nucleotide sequence represented by SEQ ID NO: 81167.

The probes having the nucleotide sequence represented by SEQ ID NOS: 148039, 148040, 148041, 148042, 148043, 148044, 148045, 148046, 148047, 148048, 148049 were selected from the ORFs having the nucleotide sequence represented by SEQ ID NO: 81168.

The probes having the nucleotide sequence represented by SEQ ID NOS: 148050, 148051, 148052, 148053, 148054, 148055, 148056, 148057, 148058, 148059, 148060 were selected from the ORFs having the nucleotide sequence represented by SEQ ID NO: 81169.

The probes having the nucleotide sequence represented by SEQ ID NOS: 148061, 148062, 148063, 148064, 148065, 148066, 148067, 148068, 148069, 148070, 148071 were selected from the ORFs having the nucleotide sequence represented by SEQ ID NO: 81170.

The probes having the nucleotide sequence represented by SEQ ID NOS: 148072, 148073, 148074, 148075, 148076, 148077, 148078, 148079, 148080, 148081, 148082 were selected from the ORFs having the nucleotide sequence represented by SEQ ID NO: 81171.

The probes having the nucleotide sequence represented by SEQ ID NOS: 148083, 148084, 148085, 148086, 148087, 148088, 148089, 148090, 148091, 148092, 148093 were selected from the ORFs having the nucleotide sequence represented by SEQ ID NO: 81172.

The probes having the nucleotide sequence represented by SEQ ID NOS: 148094, 148095, 148096, 148097, 148098, 148099, 148100, 148101, 148102, 148103, 148104 were selected from the ORFs having the nucleotide sequence represented by SEQ ID NO: 81173.

The probes having the nucleotide sequence represented by SEQ ID NOS: 148105, 148106, 148107, 148108, 148109, 148110, 148111, 148112, 148113, 148114, 148115 were selected from the ORFs having the nucleotide sequence represented by SEQ ID NO: 81174.

The probes having the nucleotide sequence represented by SEQ ID NOS: 148116, 148117, 148118, 148119, 148120, 148121, 148122, 148123, 148124, 148125, 148126 were selected from the ORFs having the nucleotide sequence represented by SEQ ID NO: 81175.

The probes having the nucleotide sequence represented by SEQ ID NOS: 148127, 148128, 148129, 148130, 148131, 148132, 148133, 148134, 148135, 148136, 148137 were selected from the ORFs having the nucleotide sequence represented by SEQ ID NO: 81176.

The probes having the nucleotide sequence represented by SEQ ID NOS: 148138, 148139, 148140, 148141, 148142, 148143, 148144, 148145, 148146, 148147, 148148 were selected from the ORFs having the nucleotide sequence represented by SEQ ID NO: 81177.

The probes having the nucleotide sequence represented by SEQ ID NOS: 148149, 148150, 148151, 148152, 148153, 148154, 148155, 148156, 148157, 148158, 148159 were selected from the ORFs having the nucleotide sequence represented by SEQ ID NO: 81178.

The probes having the nucleotide sequence represented by SEQ ID NOS: 148160, 148161, 148162, 148163, 148164, 148165, 148166, 148167, 148168, 148169, 148170 were selected from the ORFs having the nucleotide sequence represented by SEQ ID NO: 81179.

The probes having the nucleotide sequence represented by SEQ ID NOS: 148171, 148172, 148173, 148174, 148175, 148176, 148177, 148178, 148179, 148180, 148181 were selected from the ORFs having the nucleotide sequence represented by SEQ ID NO: 81180.

The probes having the nucleotide sequence represented by SEQ ID NOS: 148182, 148183, 148184, 148185, 148186, 148187, 148188, 148189, 148190, 148191, 148192 were selected from the ORFs having the nucleotide sequence represented by SEQ ID NO: 81181.

The probes having the nucleotide sequence represented by SEQ ID NOS: 148193, 148194, 148195, 148196, 148197, 148198, 148199, 148200, 148201, 148202, 148203 were selected from the ORFs having the nucleotide sequence represented by SEQ ID NO: 81182.

The probes having the nucleotide sequence represented by SEQ ID NOS: 148204, 148205, 148206, 148207, 148208, 148209, 148210, 148211, 148212, 148213, 148214 were selected from the ORFs having the nucleotide sequence represented by SEQ ID NO: 81183.

The probes having the nucleotide sequence represented by SEQ ID NOS: 148215, 148216, 148217, 148218, 148219, 148220, 148221, 148222, 148223, 148224, 148225 were selected from the ORFs having the nucleotide sequence represented by SEQ ID NO: 81184.

The probes having the nucleotide sequence represented by SEQ ID NOS: 148226, 148227, 148228, 148229, 148230, 148231, 148232, 148233, 148234, 148235, 148236 were selected from the ORFs having the nucleotide sequence represented by SEQ ID NO: 81185.

The probes having the nucleotide sequence represented by SEQ ID NOS: 148237, 148238, 148239, 148240, 148241, 148242, 148243, 148244, 148245, 148246, 148247 were selected from the ORFs having the nucleotide sequence represented by SEQ ID NO: 81186.

The probes having the nucleotide sequence represented by SEQ ID NOS: 148248, 148249, 148250, 148251, 148252, 148253, 148254, 148255, 148256, 148257, 148258 were selected from the ORFs having the nucleotide sequence represented by SEQ ID NO: 81187.

The probes having the nucleotide sequence represented by SEQ ID NOS: 148259, 148260, 148261, 148262, 148263, 148264, 148265, 148266, 148267, 148268, 148269 were selected from the ORFs having the nucleotide sequence represented by SEQ ID NO: 81188.

The probes having the nucleotide sequence represented by SEQ ID NOS: 148270, 148271, 148272, 148273, 148274, 148275, 148276, 148277, 148278, 148279, 148280 were selected from the ORFs having the nucleotide sequence represented by SEQ ID NO: 81189.

The probes having the nucleotide sequence represented by SEQ ID NOS: 148281, 148282, 148283, 148284, 148285, 148286, 148287, 148288, 148289, 148290, 148291 were selected from the ORFs having the nucleotide sequence represented by SEQ ID NO: 81190.

The probes having the nucleotide sequence represented by SEQ ID NOS: 148292, 148293, 148294, 148295, 148296, 148297, 148298, 148299, 148300, 148301, 148302 were selected from the ORFs having the nucleotide sequence represented by SEQ ID NO: 81191.

The probes having the nucleotide sequence represented by SEQ ID NOS: 148303, 148304, 148305, 148306, 148307, 148308, 148309, 148310, 148311, 148312, 148313 were selected from the ORFs having the nucleotide sequence represented by SEQ ID NO: 81192.

The probes having the nucleotide sequence represented by SEQ ID NOS: 148314, 148315, 148316, 148317, 148318, 148319, 148320, 148321, 148322, 148323, 148324 were selected from the ORFs having the nucleotide sequence represented by SEQ ID NO: 81193.

The probes having the nucleotide sequence represented by SEQ ID NOS: 148325, 148326, 148327, 148328, 148329, 148330, 148331, 148332, 148333, 148334, 148335 were selected from the ORFs having the nucleotide sequence represented by SEQ ID NO: 81194.

The probes having the nucleotide sequence represented by SEQ ID NOS: 148336, 148337, 148338, 148339, 148340, 148341, 148342, 148343, 148344, 148345, 148346 were selected from the ORFs having the nucleotide sequence represented by SEQ ID NO: 81195.

The probes having the nucleotide sequence represented by SEQ ID NOS: 148347, 148348, 148349, 148350, 148351, 148352, 148353, 148354, 148355, 148356, 148357 were selected from the ORFs having the nucleotide sequence represented by SEQ ID NO: 81196.

The probes having the nucleotide sequence represented by SEQ ID NOS: 148358, 148359, 148360, 148361, 148362, 148363, 148364, 148365, 148366, 148367, 148368 were selected from the ORFs having the nucleotide sequence represented by SEQ ID NO: 81197.

The probes having the nucleotide sequence represented by SEQ ID NOS: 148369, 148370, 148371, 148372, 148373, 148374, 148375, 148376, 148377, 148378, 148379 were selected from the ORFs having the nucleotide sequence represented by SEQ ID NO: 81198.

The probes having the nucleotide sequence represented by SEQ ID NOS: 148380, 148381, 148382, 148383, 148384, 148385, 148386, 148387, 148388, 148389, 148390 were selected from the ORFs having the nucleotide sequence represented by SEQ ID NO: 81199.

The probes having the nucleotide sequence represented by SEQ ID NOS: 148391, 148392, 148393, 148394, 148395, 148396, 148397, 148398, 148399, 148400, 148401 were selected from the ORFs having the nucleotide sequence represented by SEQ ID NO: 81200.

The probes having the nucleotide sequence represented by SEQ ID NOS: 148402, 148403, 148404, 148405, 148406, 148407, 148408, 148409, 148410, 148411, 148412 were selected from the ORFs having the nucleotide sequence represented by SEQ ID NO: 81201.

The probes having the nucleotide sequence represented by SEQ ID NOS: 148413, 148414, 148415, 148416, 148417, 148418, 148419, 148420, 148421, 148422, 148423 were selected from the ORFs having the nucleotide sequence represented by SEQ ID NO: 81202.

The probes having the nucleotide sequence represented by SEQ ID NOS: 148424, 148425, 148426, 148427, 148428, 148429, 148430, 148431, 148432, 148433, 148434 were selected from the ORFs having the nucleotide sequence represented by SEQ ID NO: 81203.

The probes having the nucleotide sequence represented by SEQ ID NOS: 148435, 148436, 148437, 148438, 148439, 148440, 148441, 148442, 148443, 148444, 148445 were selected from the ORFs having the nucleotide sequence represented by SEQ ID NO: 81204.

The probes having the nucleotide sequence represented by SEQ ID NOS: 148446, 148447, 148448, 148449, 148450, 148451, 148452, 148453, 148454, 148455, 148456 were selected from the ORFs having the nucleotide sequence represented by SEQ ID NO: 81205.

The probes having the nucleotide sequence represented by SEQ ID NOS: 148457, 148458, 148459, 148460, 148461, 148462, 148463, 148464, 148465, 148466, 148467 were selected from the ORFs having the nucleotide sequence represented by SEQ ID NO: 81206.

The probes having the nucleotide sequence represented by SEQ ID NOS: 148468, 148469, 148470, 148471, 148472, 148473, 148474, 148475, 148476, 148477, 148478 were selected from the ORFs having the nucleotide sequence represented by SEQ ID NO: 81207.

The probes having the nucleotide sequence represented by SEQ ID NOS: 148479, 148480, 148481, 148482, 148483, 148484, 148485, 148486, 148487, 148488, 148489 were selected from the ORFs having the nucleotide sequence represented by SEQ ID NO: 81208.

The probes having the nucleotide sequence represented by SEQ ID NOS: 148490, 148491, 148492, 148493, 148494, 148495, 148496, 148497, 148498, 148499, 148500 were selected from the ORFs having the nucleotide sequence represented by SEQ ID NO: 81209.

The probes having the nucleotide sequence represented by SEQ ID NOS: 148501, 148502, 148503, 148504, 148505, 148506, 148507, 148508, 148509, 148510, 148511 were selected from the ORFs having the nucleotide sequence represented by SEQ ID NO: 81210.

The probes having the nucleotide sequence represented by SEQ ID NOS: 148512, 148513, 148514, 148515, 148516, 148517, 148518, 148519, 148520, 148521, 148522 were selected from the ORFs having the nucleotide sequence represented by SEQ ID NO: 81211.

The probes having the nucleotide sequence represented by SEQ ID NOS: 148523, 148524, 148525, 148526, 148527, 148528, 148529, 148530, 148531, 148532, 148533 were selected from the ORFs having the nucleotide sequence represented by SEQ ID NO: 81212.

The probes having the nucleotide sequence represented by SEQ ID NOS: 148534, 148535, 148536, 148537, 148538, 148539, 148540, 148541, 148542, 148543, 148544 were selected from the ORFs having the nucleotide sequence represented by SEQ ID NO: 81213.

The probes having the nucleotide sequence represented by SEQ ID NOS: 148545, 148546, 148547, 148548, 148549, 148550, 148551, 148552, 148553, 148554, 148555 were selected from the ORFs having the nucleotide sequence represented by SEQ ID NO: 81214.

The probes having the nucleotide sequence represented by SEQ ID NOS: 148556, 148557, 148558, 148559, 148560, 148561, 148562, 148563, 148564, 148565, 148566 were selected from the ORFs having the nucleotide sequence represented by SEQ ID NO: 81215.

The probes having the nucleotide sequence represented by SEQ ID NOS: 148567, 148568, 148569, 148570, 148571, 148572, 148573, 148574, 148575, 148576, 148577 were selected from the ORFs having the nucleotide sequence represented by SEQ ID NO: 81216.

The probes having the nucleotide sequence represented by SEQ ID NOS: 148578, 148579, 148580, 148581, 148582, 148583, 148584, 148585, 148586, 148587, 148588 were selected from the ORFs having the nucleotide sequence represented by SEQ ID NO: 81217.

The probes having the nucleotide sequence represented by SEQ ID NOS: 148589, 148590, 148591, 148592, 148593, 148594, 148595, 148596, 148597, 148598, 148599 were selected from the ORFs having the nucleotide sequence represented by SEQ ID NO: 81218.

The probes having the nucleotide sequence represented by SEQ ID NOS: 148600, 148601, 148602, 148603, 148604, 148605, 148606, 148607, 148608, 148609, 148610 were selected from the ORFs having the nucleotide sequence represented by SEQ ID NO: 81219.

The probes having the nucleotide sequence represented by SEQ ID NOS: 148611, 148612, 148613, 148614, 148615, 148616, 148617, 148618, 148619, 148620, 148621 were selected from the ORFs having the nucleotide sequence represented by SEQ ID NO: 81220.

The probes having the nucleotide sequence represented by SEQ ID NOS: 148622, 148623, 148624, 148625, 148626, 148627, 148628, 148629, 148630, 148631, 148632 were selected from the ORFs having the nucleotide sequence represented by SEQ ID NO: 81221.

The probes having the nucleotide sequence represented by SEQ ID NOS: 148633, 148634, 148635, 148636, 148637, 148638, 148639, 148640, 148641, 148642, 148643 were selected from the ORFs having the nucleotide sequence represented by SEQ ID NO: 81222.

The probes having the nucleotide sequence represented by SEQ ID NOS: 148644, 148645, 148646, 148647, 148648, 148649, 148650, 148651, 148652, 148653, 148654 were selected from the ORFs having the nucleotide sequence represented by SEQ ID NO: 81223.

The probes having the nucleotide sequence represented by SEQ ID NOS: 148655, 148656, 148657, 148658, 148659, 148660, 148661, 148662, 148663, 148664, 148665 were selected from the ORFs having the nucleotide sequence represented by SEQ ID NO: 81224.

The probes having the nucleotide sequence represented by SEQ ID NOS: 148666, 148667, 148668, 148669, 148670, 148671, 148672, 148673, 148674, 148675, 148676 were selected from the ORFs having the nucleotide sequence represented by SEQ ID NO: 81225.

The probes having the nucleotide sequence represented by SEQ ID NOS: 148677, 148678, 148679, 148680, 148681, 148682, 148683, 148684, 148685, 148686, 148687 were selected from the ORFs having the nucleotide sequence represented by SEQ ID NO: 81226.

The probes having the nucleotide sequence represented by SEQ ID NOS: 148688, 148689, 148690, 148691, 148692, 148693, 148694, 148695, 148696, 148697, 148698 were selected from the ORFs having the nucleotide sequence represented by SEQ ID NO: 81227.

The probes having the nucleotide sequence represented by SEQ ID NOS: 148699, 148700, 148701, 148702, 148703, 148704, 148705, 148706, 148707, 148708, 148709 were selected from the ORFs having the nucleotide sequence represented by SEQ ID NO: 81228.

The probes having the nucleotide sequence represented by SEQ ID NOS: 148710, 148711, 148712, 148713, 148714, 148715, 148716, 148717, 148718, 148719, 148720 were selected from the ORFs having the nucleotide sequence represented by SEQ ID NO: 81229.

The probes having the nucleotide sequence represented by SEQ ID NOS: 148721, 148722, 148723, 148724, 148725, 148726, 148727, 148728, 148729, 148730, 148731 were selected from the ORFs having the nucleotide sequence represented by SEQ ID NO: 81230.

The probes having the nucleotide sequence represented by SEQ ID NOS: 148732, 148733, 148734, 148735, 148736, 148737, 148738, 148739, 148740, 148741, 148742 were selected from the ORFs having the nucleotide sequence represented by SEQ ID NO: 81231.

The probes having the nucleotide sequence represented by SEQ ID NOS: 148743, 148744, 148745, 148746, 148747, 148748, 148749, 148750, 148751, 148752, 148753 were selected from the ORFs having the nucleotide sequence represented by SEQ ID NO: 81232.

The probes having the nucleotide sequence represented by SEQ ID NOS: 148754, 148755, 148756, 148757, 148758, 148759, 148760, 148761, 148762, 148763, 148764 were selected from the ORFs having the nucleotide sequence represented by SEQ ID NO: 81233.

The probes having the nucleotide sequence represented by SEQ ID NOS: 148765, 148766, 148767, 148768, 148769, 148770, 148771, 148772, 148773, 148774, 148775 were selected from the ORFs having the nucleotide sequence represented by SEQ ID NO: 81234.

The probes having the nucleotide sequence represented by SEQ ID NOS: 148776, 148777, 148778, 148779, 148780, 148781, 148782, 148783, 148784, 148785, 148786 were selected from the ORFs having the nucleotide sequence represented by SEQ ID NO: 81235.

The probes having the nucleotide sequence represented by SEQ ID NOS: 148787, 148788, 148789, 148790, 148791, 148792, 148793, 148794, 148795, 148796, 148797 were selected from the ORFs having the nucleotide sequence represented by SEQ ID NO: 81236.

The probes having the nucleotide sequence represented by SEQ ID NOS: 148798, 148799, 148800, 148801, 148802, 148803, 148804, 148805, 148806, 148807, 148808 were selected from the ORFs having the nucleotide sequence represented by SEQ ID NO: 81237.

The probes having the nucleotide sequence represented by SEQ ID NOS: 148809, 148810, 148811, 148812, 148813, 148814, 148815, 148816, 148817, 148818, 148819 were selected from the ORFs having the nucleotide sequence represented by SEQ ID NO: 81238.

The probes having the nucleotide sequence represented by SEQ ID NOS: 148820, 148821, 148822, 148823, 148824, 148825, 148826, 148827, 148828, 148829, 148830 were selected from the ORFs having the nucleotide sequence represented by SEQ ID NO: 81239.

The probes having the nucleotide sequence represented by SEQ ID NOS: 148831, 148832, 148833, 148834, 148835, 148836, 148837, 148838, 148839, 148840, 148841 were selected from the ORFs having the nucleotide sequence represented by SEQ ID NO: 81240.

The probes having the nucleotide sequence represented by SEQ ID NOS: 148842, 148843, 148844, 148845, 148846, 148847, 148848, 148849, 148850, 148851, 148852 were selected from the ORFs having the nucleotide sequence represented by SEQ ID NO: 81241.

The probes having the nucleotide sequence represented by SEQ ID NOS: 148853, 148854, 148855, 148856, 148857, 148858, 148859, 148860, 148861, 148862, 148863 were selected from the ORFs having the nucleotide sequence represented by SEQ ID NO: 81242.

The probes having the nucleotide sequence represented by SEQ ID NOS: 148864, 148865, 148866, 148867, 148868, 148869, 148870, 148871, 148872, 148873, 148874 were selected from the ORFs having the nucleotide sequence represented by SEQ ID NO: 81243.

The probes having the nucleotide sequence represented by SEQ ID NOS: 148875, 148876, 148877, 148878, 148879, 148880, 148881, 148882, 148883, 148884, 148885 were selected from the ORFs having the nucleotide sequence represented by SEQ ID NO: 81244.

The probes having the nucleotide sequence represented by SEQ ID NOS: 148886, 148887, 148888, 148889, 148890, 148891, 148892, 148893, 148894, 148895, 148896 were selected from the ORFs having the nucleotide sequence represented by SEQ ID NO: 81245.

The probes having the nucleotide sequence represented by SEQ ID NOS: 148897, 148898, 148899, 148900, 148901, 148902, 148903, 148904, 148905, 148906, 148907 were selected from the ORFs having the nucleotide sequence represented by SEQ ID NO: 81246.

The probes having the nucleotide sequence represented by SEQ ID NOS: 148908, 148909, 148910, 148911, 148912, 148913, 148914, 148915, 148916, 148917, 148918 were selected from the ORFs having the nucleotide sequence represented by SEQ ID NO: 81247.

The probes having the nucleotide sequence represented by SEQ ID NOS: 148919, 148920, 148921, 148922, 148923, 148924, 148925, 148926, 148927, 148928, 148929 were selected from the ORFs having the nucleotide sequence represented by SEQ ID NO: 81248.

The probes having the nucleotide sequence represented by SEQ ID NOS: 148930, 148931, 148932, 148933, 148934, 148935, 148936, 148937, 148938, 148939, 148940 were selected from the ORFs having the nucleotide sequence represented by SEQ ID NO: 81249.

The probes having the nucleotide sequence represented by SEQ ID NOS: 148941, 148942, 148943, 148944, 148945, 148946, 148947, 148948, 148949, 148950, 148951 were selected from the ORFs having the nucleotide sequence represented by SEQ ID NO: 81250.

The probes having the nucleotide sequence represented by SEQ ID NOS: 148952, 148953, 148954, 148955, 148956, 148957, 148958, 148959, 148960, 148961, 148962 were selected from the ORFs having the nucleotide sequence represented by SEQ ID NO: 81251.

The probes having the nucleotide sequence represented by SEQ ID NOS: 148963, 148964, 148965, 148966, 148967, 148968, 148969, 148970, 148971, 148972, 148973 were selected from the ORFs having the nucleotide sequence represented by SEQ ID NO: 81252.

The probes having the nucleotide sequence represented by SEQ ID NOS: 148974, 148975, 148976, 148977, 148978, 148979, 148980, 148981, 148982, 148983, 148984 were selected from the ORFs having the nucleotide sequence represented by SEQ ID NO: 81253.

The probes having the nucleotide sequence represented by SEQ ID NOS: 148985, 148986, 148987, 148988, 148989, 148990, 148991, 148992, 148993, 148994, 148995 were selected from the ORFs having the nucleotide sequence represented by SEQ ID NO: 81254.

The probes having the nucleotide sequence represented by SEQ ID NOS: 148996, 148997, 148998, 148999, 149000, 149001, 149002, 149003, 149004, 149005, 149006 were selected from the ORFs having the nucleotide sequence represented by SEQ ID NO: 81255.

The probes having the nucleotide sequence represented by SEQ ID NOS: 149007, 149008, 149009, 149010, 149011, 149012, 149013, 149014, 149015, 149016, 149017 were selected from the ORFs having the nucleotide sequence represented by SEQ ID NO: 81256.

The probes having the nucleotide sequence represented by SEQ ID NOS: 149018, 149019, 149020, 149021, 149022, 149023, 149024, 149025, 149026, 149027, 149028 were selected from the ORFs having the nucleotide sequence represented by SEQ ID NO: 81257.

The probes having the nucleotide sequence represented by SEQ ID NOS: 149029, 149030, 149031, 149032, 149033, 149034, 149035, 149036, 149037, 149038, 149039 were selected from the ORFs having the nucleotide sequence represented by SEQ ID NO: 81258.

The probes having the nucleotide sequence represented by SEQ ID NOS: 149040, 149041, 149042, 149043, 149044, 149045, 149046, 149047, 149048, 149049, 149050 were selected from the ORFs having the nucleotide sequence represented by SEQ ID NO: 81259.

The probes having the nucleotide sequence represented by SEQ ID NOS: 149051, 149052, 149053, 149054, 149055, 149056, 149057, 149058, 149059, 149060, 149061 were selected from the ORFs having the nucleotide sequence represented by SEQ ID NO: 81260.

The probes having the nucleotide sequence represented by SEQ ID NOS: 149062, 149063, 149064, 149065, 149066, 149067, 149068, 149069, 149070, 149071 were selected from the ORFs having the nucleotide sequence represented by SEQ ID NO: 81261.

The probes having the nucleotide sequence represented by SEQ ID NOS: 149072, 149073, 149074, 149075, 149076, 149077, 149078, 149079, 149080, 149081, 149082 were selected from the ORFs having the nucleotide sequence represented by SEQ ID NO: 81262.

The probes having the nucleotide sequence represented by SEQ ID NOS: 149083, 149084, 149085, 149086, 149087, 149088, 149089, 149090, 149091, 149092, 149093 were selected from the ORFs having the nucleotide sequence represented by SEQ ID NO: 81263.

The probes having the nucleotide sequence represented by SEQ ID NOS: 149094, 149095, 149096, 149097, 149098, 149099, 149100, 149101, 149102, 149103, 149104 were selected from the ORFs having the nucleotide sequence represented by SEQ ID NO: 81264.

The probes having the nucleotide sequence represented by SEQ ID NOS: 149105, 149106, 149107, 149108, 149109, 149110, 149111, 149112, 149113, 149114, 149115 were selected from the ORFs having the nucleotide sequence represented by SEQ ID NO: 81265.

The probes having the nucleotide sequence represented by SEQ ID NOS: 149116, 149117, 149118, 149119, 149120, 149121, 149122, 149123, 149124, 149125, 149126 were selected from the ORFs having the nucleotide sequence represented by SEQ ID NO: 81266.

The probes having the nucleotide sequence represented by SEQ ID NOS: 149127, 149128, 149129, 149130, 149131, 149132, 149133, 149134, 149135, 149136, 149137 were selected from the ORFs having the nucleotide sequence represented by SEQ ID NO: 81267.

The probes having the nucleotide sequence represented by SEQ ID NOS: 149138, 149139, 149140, 149141, 149142, 149143, 149144, 149145, 149146, 149147, 149148 were selected from the ORFs having the nucleotide sequence represented by SEQ ID NO: 81268.

The probes having the nucleotide sequence represented by SEQ ID NOS: 149149, 149150, 149151, 149152, 149153, 149154, 149155, 149156, 149157, 149158, 149159 were selected from the ORFs having the nucleotide sequence represented by SEQ ID NO: 81269.

The probes having the nucleotide sequence represented by SEQ ID NOS: 149160, 149161, 149162, 149163, 149164, 149165, 149166, 149167, 149168, 149169, 149170 were selected from the ORFs having the nucleotide sequence represented by SEQ ID NO: 81270.

The probes having the nucleotide sequence represented by SEQ ID NOS: 149171, 149172, 149173, 149174, 149175, 149176, 149177, 149178, 149179, 149180, 149181 were selected from the ORFs having the nucleotide sequence represented by SEQ ID NO: 81271.

The probes having the nucleotide sequence represented by SEQ ID NOS: 149182, 149183, 149184, 149185, 149186, 149187, 149188, 149189, 149190, 149191, 149192 were selected from the ORFs having the nucleotide sequence represented by SEQ ID NO: 81272.

The probes having the nucleotide sequence represented by SEQ ID NOS: 149193, 149194, 149195, 149196, 149197, 149198, 149199, 149200, 149201, 149202, 149203 were selected from the ORFs having the nucleotide sequence represented by SEQ ID NO: 81273.

The probes having the nucleotide sequence represented by SEQ ID NOS: 149204, 149205, 149206, 149207, 149208, 149209, 149210, 149211, 149212, 149213, 149214 were selected from the ORFs having the nucleotide sequence represented by SEQ ID NO: 81274.

The probes having the nucleotide sequence represented by SEQ ID NOS: 149215, 149216, 149217, 149218, 149219, 149220, 149221, 149222, 149223, 149224, 149225 were selected from the ORFs having the nucleotide sequence represented by SEQ ID NO: 81275.

The probes having the nucleotide sequence represented by SEQ ID NOS: 149226, 149227, 149228, 149229, 149230, 149231, 149232, 149233, 149234, 149235, 149236 were selected from the ORFs having the nucleotide sequence represented by SEQ ID NO: 81276.

The probes having the nucleotide sequence represented by SEQ ID NOS: 149237, 149238, 149239, 149240, 149241, 149242, 149243, 149244, 149245, 149246, 149247 were selected from the ORFs having the nucleotide sequence represented by SEQ ID NO: 81277.

The probes having the nucleotide sequence represented by SEQ ID NOS: 149248, 149249, 149250, 149251, 149252, 149253, 149254, 149255, 149256, 149257, 149258 were selected from the ORFs having the nucleotide sequence represented by SEQ ID NO: 81278.

The probes having the nucleotide sequence represented by SEQ ID NOS: 149259, 149260, 149261, 149262, 149263, 149264, 149265, 149266, 149267, 149268, 149269 were selected from the ORFs having the nucleotide sequence represented by SEQ ID NO: 81279.

The probes having the nucleotide sequence represented by SEQ ID NOS: 149270, 149271, 149272, 149273, 149274, 149275, 149276, 149277, 149278, 149279, 149280 were selected from the ORFs having the nucleotide sequence represented by SEQ ID NO: 81280.

The probes having the nucleotide sequence represented by SEQ ID NOS: 149281, 149282, 149283, 149284, 149285, 149286, 149287, 149288, 149289, 149290, 149291 were selected from the ORFs having the nucleotide sequence represented by SEQ ID NO: 81281.

The probes having the nucleotide sequence represented by SEQ ID NOS: 149292, 149293, 149294, 149295, 149296, 149297, 149298, 149299, 149300, 149301, 149302 were selected from the ORFs having the nucleotide sequence represented by SEQ ID NO: 81282.

The probes having the nucleotide sequence represented by SEQ ID NOS: 149303, 149304, 149305, 149306, 149307, 149308, 149309, 149310, 149311, 149312, 149313 were selected from the ORFs having the nucleotide sequence represented by SEQ ID NO: 81283.

The probes having the nucleotide sequence represented by SEQ ID NOS: 149314, 149315, 149316, 149317, 149318, 149319, 149320, 149321, 149322, 149323, 149324 were selected from the ORFs having the nucleotide sequence represented by SEQ ID NO: 81284.

The probes having the nucleotide sequence represented by SEQ ID NOS: 149325, 149326, 149327, 149328, 149329, 149330, 149331, 149332, 149333, 149334, 149335 were selected from the ORFs having the nucleotide sequence represented by SEQ ID NO: 81285.

The probes having the nucleotide sequence represented by SEQ ID NOS: 149336, 149337, 149338, 149339, 149340, 149341, 149342, 149343, 149344, 149345, 149346 were selected from the ORFs having the nucleotide sequence represented by SEQ ID NO: 81286.

The probes having the nucleotide sequence represented by SEQ ID NOS: 149347, 149348, 149349, 149350, 149351, 149352, 149353, 149354, 149355, 149356, 149357 were selected from the ORFs having the nucleotide sequence represented by SEQ ID NO: 81287.

The probes having the nucleotide sequence represented by SEQ ID NOS: 149358, 149359, 149360, 149361, 149362, 149363, 149364, 149365, 149366, 149367, 149368 were selected from the ORFs having the nucleotide sequence represented by SEQ ID NO: 81288.

The probes having the nucleotide sequence represented by SEQ ID NOS: 149369, 149370, 149371, 149372, 149373, 149374, 149375, 149376, 149377, 149378, 149379 were selected from the ORFs having the nucleotide sequence represented by SEQ ID NO: 81289.

The probes having the nucleotide sequence represented by SEQ ID NOS: 149380, 149381, 149382, 149383, 149384, 149385, 149386, 149387, 149388, 149389, 149390 were selected from the ORFs having the nucleotide sequence represented by SEQ ID NO: 81290.

The probes having the nucleotide sequence represented by SEQ ID NOS: 149391, 149392, 149393, 149394, 149395, 149396, 149397, 149398, 149399, 149400, 149401 were selected from the ORFs having the nucleotide sequence represented by SEQ ID NO: 81291.

The probes having the nucleotide sequence represented by SEQ ID NOS: 149402, 149403, 149404, 149405, 149406, 149407, 149408, 149409, 149410, 149411, 149412 were selected from the ORFs having the nucleotide sequence represented by SEQ ID NO: 81292.

The probes having the nucleotide sequence represented by SEQ ID NOS: 149413, 149414, 149415, 149416, 149417, 149418, 149419, 149420, 149421, 149422, 149423 were selected from the ORFs having the nucleotide sequence represented by SEQ ID NO: 81293.

The probes having the nucleotide sequence represented by SEQ ID NOS: 149424, 149425, 149426, 149427, 149428, 149429, 149430, 149431, 149432, 149433, 149434 were selected from the ORFs having the nucleotide sequence represented by SEQ ID NO: 81294.

The probes having the nucleotide sequence represented by SEQ ID NOS: 149435, 149436, 149437, 149438, 149439, 149440, 149441, 149442, 149443, 149444, 149445 were selected from the ORFs having the nucleotide sequence represented by SEQ ID NO: 81295.

The probes having the nucleotide sequence represented by SEQ ID NOS: 149446, 149447, 149448, 149449, 149450, 149451, 149452, 149453, 149454, 149455, 149456 were selected from the ORFs having the nucleotide sequence represented by SEQ ID NO: 81296.

The probes having the nucleotide sequence represented by SEQ ID NOS: 149457, 149458, 149459, 149460, 149461, 149462, 149463, 149464, 149465, 149466, 149467 were selected from the ORFs having the nucleotide sequence represented by SEQ ID NO: 81297.

The probes having the nucleotide sequence represented by SEQ ID NOS: 149468, 149469, 149470, 149471, 149472, 149473, 149474, 149475, 149476, 149477, 149478 were selected from the ORFs having the nucleotide sequence represented by SEQ ID NO: 81298.

The probes having the nucleotide sequence represented by SEQ ID NOS: 149479, 149480, 149481, 149482, 149483, 149484, 149485, 149486, 149487, 149488, 149489 were selected from the ORFs having the nucleotide sequence represented by SEQ ID NO: 81299.

The probes having the nucleotide sequence represented by SEQ ID NOS: 149490, 149491, 149492, 149493, 149494, 149495, 149496, 149497, 149498, 149499, 149500 were selected from the ORFs having the nucleotide sequence represented by SEQ ID NO: 81300.

The probes having the nucleotide sequence represented by SEQ ID NOS: 149501, 149502, 149503, 149504, 149505, 149506, 149507, 149508, 149509, 149510, 149511 were selected from the ORFs having the nucleotide sequence represented by SEQ ID NO: 81301.

The probes having the nucleotide sequence represented by SEQ ID NOS: 149512, 149513, 149514, 149515, 149516, 149517, 149518, 149519, 149520, 149521, 149522 were selected from the ORFs having the nucleotide sequence represented by SEQ ID NO: 81302.

The probes having the nucleotide sequence represented by SEQ ID NOS: 149523, 149524, 149525, 149526, 149527, 149528, 149529, 149530, 149531, 149532, 149533 were selected from the ORFs having the nucleotide sequence represented by SEQ ID NO: 81303.

The probes having the nucleotide sequence represented by SEQ ID NOS: 149534, 149535, 149536, 149537, 149538, 149539, 149540, 149541, 149542, 149543, 149544 were selected from the ORFs having the nucleotide sequence represented by SEQ ID NO: 81304.

The probes having the nucleotide sequence represented by SEQ ID NOS: 149545, 149546, 149547, 149548, 149549, 149550, 149551, 149552, 149553, 149554, 149555 were selected from the ORFs having the nucleotide sequence represented by SEQ ID NO: 81305.

The probes having the nucleotide sequence represented by SEQ ID NOS: 149556, 149557, 149558, 149559, 149560, 149561, 149562, 149563, 149564, 149565, 149566 were selected from the ORFs having the nucleotide sequence represented by SEQ ID NO: 81306.

The probes having the nucleotide sequence represented by SEQ ID NOS: 149567, 149568, 149569, 149570, 149571, 149572, 149573, 149574, 149575, 149576, 149577 were selected from the ORFs having the nucleotide sequence represented by SEQ ID NO: 81307.

The probes having the nucleotide sequence represented by SEQ ID NOS: 149578, 149579, 149580, 149581, 149582, 149583, 149584, 149585, 149586, 149587, 149588 were selected from the ORFs having the nucleotide sequence represented by SEQ ID NO: 81308.

The probes having the nucleotide sequence represented by SEQ ID NOS: 149589, 149590, 149591, 149592, 149593, 149594, 149595, 149596, 149597, 149598, 149599 were selected from the ORFs having the nucleotide sequence represented by SEQ ID NO: 81309.

The probes having the nucleotide sequence represented by SEQ ID NOS: 149600, 149601, 149602, 149603, 149604, 149605, 149606, 149607, 149608, 149609, 149610 were selected from the ORFs having the nucleotide sequence represented by SEQ ID NO: 81310.

The probes having the nucleotide sequence represented by SEQ ID NOS: 149611, 149612, 149613, 149614, 149615, 149616, 149617, 149618, 149619, 149620, 149621 were selected from the ORFs having the nucleotide sequence represented by SEQ ID NO: 81311.

The probes having the nucleotide sequence represented by SEQ ID NOS: 149622, 149623, 149624, 149625, 149626, 149627, 149628, 149629, 149630, 149631, 149632 were selected from the ORFs having the nucleotide sequence represented by SEQ ID NO: 81312.

The probes having the nucleotide sequence represented by SEQ ID NOS: 149633, 149634, 149635, 149636, 149637, 149638, 149639, 149640, 149641, 149642, 149643 were selected from the ORFs having the nucleotide sequence represented by SEQ ID NO: 81313.

The probes having the nucleotide sequence represented by SEQ ID NOS: 149644, 149645, 149646, 149647, 149648, 149649, 149650, 149651, 149652, 149653, 149654 were selected from the ORFs having the nucleotide sequence represented by SEQ ID NO: 81314.

The probes having the nucleotide sequence represented by SEQ ID NOS: 149655, 149656, 149657, 149658, 149659, 149660, 149661, 149662, 149663, 149664, 149665 were selected from the ORFs having the nucleotide sequence represented by SEQ ID NO: 81315.

The probes having the nucleotide sequence represented by SEQ ID NOS: 149666, 149667, 149668, 149669, 149670, 149671, 149672, 149673, 149674, 149675, 149676 were selected from the ORFs having the nucleotide sequence represented by SEQ ID NO: 81316.

The probes having the nucleotide sequence represented by SEQ ID NOS: 149677, 149678, 149679, 149680, 149681, 149682, 149683, 149684, 149685, 149686, 149687 were selected from the ORFs having the nucleotide sequence represented by SEQ ID NO: 81317.

The probes having the nucleotide sequence represented by SEQ ID NOS: 149688, 149689, 149690, 149691, 149692, 149693, 149694, 149695, 149696, 149697, 149698 were selected from the ORFs having the nucleotide sequence represented by SEQ ID NO: 81318.

The probes having the nucleotide sequence represented by SEQ ID NOS: 149699, 149700, 149701, 149702, 149703, 149704, 149705, 149706, 149707, 149708, 149709 were selected from the ORFs having the nucleotide sequence represented by SEQ ID NO: 81319.

The probes having the nucleotide sequence represented by SEQ ID NOS: 149710, 149711, 149712, 149713, 149714, 149715, 149716, 149717, 149718, 149719, 149720 were selected from the ORFs having the nucleotide sequence represented by SEQ ID NO: 81320.

The probes having the nucleotide sequence represented by SEQ ID NOS: 149721, 149722, 149723, 149724, 149725, 149726, 149727, 149728, 149729, 149730, 149731 were selected from the ORFs having the nucleotide sequence represented by SEQ ID NO: 81321.

The probes having the nucleotide sequence represented by SEQ ID NOS: 149732, 149733, 149734, 149735, 149736, 149737, 149738, 149739, 149740, 149741, 149742 were selected from the ORFs having the nucleotide sequence represented by SEQ ID NO: 81322.

The probes having the nucleotide sequence represented by SEQ ID NOS: 149743, 149744, 149745, 149746, 149747, 149748, 149749, 149750, 149751, 149752, 149753 were selected from the ORFs having the nucleotide sequence represented by SEQ ID NO: 81323.

The probes having the nucleotide sequence represented by SEQ ID NOS: 149754, 149755, 149756, 149757, 149758, 149759, 149760, 149761, 149762, 149763, 149764 were selected from the ORFs having the nucleotide sequence represented by SEQ ID NO: 81324.

The probes having the nucleotide sequence represented by SEQ ID NOS: 149765, 149766, 149767, 149768, 149769, 149770, 149771, 149772, 149773, 149774, 149775 were selected from the ORFs having the nucleotide sequence represented by SEQ ID NO: 81325.

The probes having the nucleotide sequence represented by SEQ ID NOS: 149776, 149777, 149778, 149779, 149780, 149781, 149782, 149783, 149784, 149785, 149786 were selected from the ORFs having the nucleotide sequence represented by SEQ ID NO: 81326.

The probes having the nucleotide sequence represented by SEQ ID NOS: 149787, 149788, 149789, 149790, 149791, 149792, 149793, 149794, 149795, 149796, 149797 were selected from the ORFs having the nucleotide sequence represented by SEQ ID NO: 81327.

The probes having the nucleotide sequence represented by SEQ ID NOS: 149798, 149799, 149800, 149801, 149802, 149803, 149804, 149805, 149806, 149807, 149808 were selected from the ORFs having the nucleotide sequence represented by SEQ ID NO: 81328.

The probes having the nucleotide sequence represented by SEQ ID NOS: 149809, 149810, 149811, 149812, 149813, 149814, 149815, 149816, 149817, 149818, 149819 were selected from the ORFs having the nucleotide sequence represented by SEQ ID NO: 81329.

The probes having the nucleotide sequence represented by SEQ ID NOS: 149820, 149821, 149822, 149823, 149824, 149825, 149826, 149827, 149828, 149829, 149830 were selected from the ORFs having the nucleotide sequence represented by SEQ ID NO: 81330.

The probes having the nucleotide sequence represented by SEQ ID NOS: 149831, 149832, 149833, 149834, 149835, 149836, 149837, 149838, 149839, 149840, 149841 were selected from the ORFs having the nucleotide sequence represented by SEQ ID NO: 81331.

The probes having the nucleotide sequence represented by SEQ ID NOS: 149842, 149843, 149844, 149845, 149846, 149847, 149848, 149849, 149850, 149851, 149852 were selected from the ORFs having the nucleotide sequence represented by SEQ ID NO: 81332.

The probes having the nucleotide sequence represented by SEQ ID NOS: 149853, 149854, 149855, 149856, 149857, 149858, 149859, 149860, 149861, 149862, 149863 were selected from the ORFs having the nucleotide sequence represented by SEQ ID NO: 81333.

The probes having the nucleotide sequence represented by SEQ ID NOS: 149864, 149865, 149866, 149867, 149868, 149869, 149870, 149871, 149872, 149873, 149874 were selected from the ORFs having the nucleotide sequence represented by SEQ ID NO: 81334.

The probes having the nucleotide sequence represented by SEQ ID NOS: 149875, 149876, 149877, 149878, 149879, 149880, 149881, 149882, 149883, 149884, 149885 were selected from the ORFs having the nucleotide sequence represented by SEQ ID NO: 81335.

The probes having the nucleotide sequence represented by SEQ ID NOS: 149886, 149887, 149888, 149889, 149890, 149891, 149892, 149893, 149894, 149895, 149896 were selected from the ORFs having the nucleotide sequence represented by SEQ ID NO: 81336.

The probes having the nucleotide sequence represented by SEQ ID NOS: 149897, 149898, 149899, 149900, 149901, 149902, 149903, 149904, 149905, 149906, 149907 were selected from the ORFs having the nucleotide sequence represented by SEQ ID NO: 81337.

The probes having the nucleotide sequence represented by SEQ ID NOS: 149908, 149909, 149910, 149911, 149912, 149913, 149914, 149915, 149916, 149917, 149918 were selected from the ORFs having the nucleotide sequence represented by SEQ ID NO: 81338.

The probes having the nucleotide sequence represented by SEQ ID NOS: 149919, 149920, 149921, 149922, 149923, 149924, 149925, 149926, 149927, 149928, 149929 were selected from the ORFs having the nucleotide sequence represented by SEQ ID NO: 81339.

The probes having the nucleotide sequence represented by SEQ ID NOS: 149930, 149931, 149932, 149933, 149934, 149935, 149936, 149937, 149938, 149939, 149940 were selected from the ORFs having the nucleotide sequence represented by SEQ ID NO: 81340.

The probes having the nucleotide sequence represented by SEQ ID NOS: 149941, 149942, 149943, 149944, 149945, 149946, 149947, 149948, 149949, 149950, 149951 were selected from the ORFs having the nucleotide sequence represented by SEQ ID NO: 81341.

The probes having the nucleotide sequence represented by SEQ ID NOS: 149952, 149953, 149954, 149955, 149956, 149957, 149958, 149959, 149960, 149961, 149962 were selected from the ORFs having the nucleotide sequence represented by SEQ ID NO: 81342.

The probes having the nucleotide sequence represented by SEQ ID NOS: 149963, 149964, 149965, 149966, 149967, 149968, 149969, 149970, 149971, 149972, 149973 were selected from the ORFs having the nucleotide sequence represented by SEQ ID NO: 81343.

The probes having the nucleotide sequence represented by SEQ ID NOS: 149974, 149975, 149976, 149977, 149978, 149979, 149980, 149981, 149982, 149983, 149984 were selected from the ORFs having the nucleotide sequence represented by SEQ ID NO: 81344.

The probes having the nucleotide sequence represented by SEQ ID NOS: 149985, 149986, 149987, 149988, 149989, 149990, 149991, 149992, 149993, 149994, 149995 were selected from the ORFs having the nucleotide sequence represented by SEQ ID NO: 81345.

The probes having the nucleotide sequence represented by SEQ ID NOS: 149996, 149997, 149998, 149999, 150000, 150001, 150002, 150003, 150004, 150005, 150006 were selected from the ORFs having the nucleotide sequence represented by SEQ ID NO: 81346.

The probes having the nucleotide sequence represented by SEQ ID NOS: 150007, 150008, 150009, 150010, 150011, 150012, 150013, 150014, 150015, 150016, 150017 were selected from the ORFs having the nucleotide sequence represented by SEQ ID NO: 81347.

The probes having the nucleotide sequence represented by SEQ ID NOS: 150018, 150019, 150020, 150021, 150022, 150023, 150024, 150025, 150026, 150027, 150028 were selected from the ORFs having the nucleotide sequence represented by SEQ ID NO: 81348.

The probes having the nucleotide sequence represented by SEQ ID NOS: 150029, 150030, 150031, 150032, 150033, 150034, 150035, 150036, 150037, 150038, 150039 were selected from the ORFs having the nucleotide sequence represented by SEQ ID NO: 81349.

The probes having the nucleotide sequence represented by SEQ ID NOS: 150040, 150041, 150042, 150043, 150044, 150045, 150046, 150047, 150048, 150049, 150050 were selected from the ORFs having the nucleotide sequence represented by SEQ ID NO: 81350.

The probes having the nucleotide sequence represented by SEQ ID NOS: 150051, 150052, 150053, 150054, 150055, 150056, 150057, 150058, 150059, 150060, 150061 were selected from the ORFs having the nucleotide sequence represented by SEQ ID NO: 81351.

The probes having the nucleotide sequence represented by SEQ ID NOS: 150062, 150063, 150064, 150065, 150066, 150067, 150068, 150069, 150070, 150071, 150072 were selected from the ORFs having the nucleotide sequence represented by SEQ ID NO: 81352.

The probes having the nucleotide sequence represented by SEQ ID NOS: 150073, 150074, 150075, 150076, 150077, 150078, 150079, 150080, 150081, 150082, 150083 were selected from the ORFs having the nucleotide sequence represented by SEQ ID NO: 81353.

The probes having the nucleotide sequence represented by SEQ ID NOS: 150084, 150085, 150086, 150087, 150088, 150089, 150090, 150091, 150092, 150093, 150094 were selected from the ORFs having the nucleotide sequence represented by SEQ ID NO: 81354.

The probes having the nucleotide sequence represented by SEQ ID NOS: 150095, 150096, 150097, 150098, 150099, 150100, 150101, 150102, 150103, 150104, 150105 were selected from the ORFs having the nucleotide sequence represented by SEQ ID NO: 81355.

The probes having the nucleotide sequence represented by SEQ ID NOS: 150106, 150107, 150108, 150109, 150110, 150111, 150112, 150113, 150114, 150115, 150116 were selected from the ORFs having the nucleotide sequence represented by SEQ ID NO: 81356.

The probes having the nucleotide sequence represented by SEQ ID NOS: 150117, 150118, 150119, 150120, 150121, 150122, 150123, 150124, 150125, 150126, 150127 were selected from the ORFs having the nucleotide sequence represented by SEQ ID NO: 81357.

The probes having the nucleotide sequence represented by SEQ ID NOS: 150128, 150129, 150130, 150131, 150132, 150133, 150134, 150135, 150136, 150137, 150138 were selected from the ORFs having the nucleotide sequence represented by SEQ ID NO: 81358.

The probes having the nucleotide sequence represented by SEQ ID NOS: 150139, 150140, 150141, 150142, 150143, 150144, 150145, 150146, 150147, 150148, 150149 were selected from the ORFs having the nucleotide sequence represented by SEQ ID NO: 81359.

The probes having the nucleotide sequence represented by SEQ ID NOS: 150150, 150151, 150152, 150153, 150154, 150155, 150156, 150157, 150158, 150159, 150160 were selected from the ORFs having the nucleotide sequence represented by SEQ ID NO: 81360.

The probes having the nucleotide sequence represented by SEQ ID NOS: 150161, 150162, 150163, 150164, 150165, 150166, 150167, 150168, 150169, 150170, 150171 were selected from the ORFs having the nucleotide sequence represented by SEQ ID NO: 81361.

The probes having the nucleotide sequence represented by SEQ ID NOS: 150172, 150173, 150174, 150175, 150176, 150177, 150178, 150179, 150180, 150181, 150182 were selected from the ORFs having the nucleotide sequence represented by SEQ ID NO: 81362.

The probes having the nucleotide sequence represented by SEQ ID NOS: 150183, 150184, 150185, 150186, 150187, 150188, 150189, 150190, 150191, 150192, 150193 were selected from the ORFs having the nucleotide sequence represented by SEQ ID NO: 81363.

The probes having the nucleotide sequence represented by SEQ ID NOS: 150194, 150195, 150196, 150197, 150198, 150199, 150200, 150201, 150202, 150203, 150204 were selected from the ORFs having the nucleotide sequence represented by SEQ ID NO: 81364.

The probes having the nucleotide sequence represented by SEQ ID NOS: 150205, 150206, 150207, 150208, 150209, 150210, 150211, 150212, 150213, 150214, 150215 were selected from the ORFs having the nucleotide sequence represented by SEQ ID NO: 81365.

The probes having the nucleotide sequence represented by SEQ ID NOS: 150216, 150217, 150218, 150219, 150220, 150221, 150222, 150223, 150224, 150225, 150226 were selected from the ORFs having the nucleotide sequence represented by SEQ ID NO: 81366.

The probes having the nucleotide sequence represented by SEQ ID NOS: 150227, 150228, 150229, 150230, 150231, 150232, 150233, 150234, 150235, 150236, 150237 were selected from the ORFs having the nucleotide sequence represented by SEQ ID NO: 81367.

The probes having the nucleotide sequence represented by SEQ ID NOS: 150238, 150239, 150240, 150241, 150242, 150243, 150244, 150245, 150246, 150247, 150248 were selected from the ORFs having the nucleotide sequence represented by SEQ ID NO: 81368.

The probes having the nucleotide sequence represented by SEQ ID NOS: 150249, 150250, 150251, 150252, 150253, 150254, 150255, 150256, 150257, 150258, 150259 were selected from the ORFs having the nucleotide sequence represented by SEQ ID NO: 81369.

The probes having the nucleotide sequence represented by SEQ ID NOS: 150260, 150261, 150262, 150263, 150264, 150265, 150266, 150267, 150268, 150269, 150270 were selected from the ORFs having the nucleotide sequence represented by SEQ ID NO: 81370.

The probes having the nucleotide sequence represented by SEQ ID NOS: 150271, 150272, 150273, 150274, 150275, 150276, 150277, 150278, 150279, 150280, 150281 were selected from the ORFs having the nucleotide sequence represented by SEQ ID NO: 81371.

The probes having the nucleotide sequence represented by SEQ ID NOS: 150282, 150283, 150284, 150285, 150286, 150287, 150288, 150289, 150290, 150291, 150292 were selected from the ORFs having the nucleotide sequence represented by SEQ ID NO: 81372.

The probes having the nucleotide sequence represented by SEQ ID NOS: 150293, 150294, 150295, 150296, 150297, 150298, 150299, 150300, 150301, 150302, 150303 were selected from the ORFs having the nucleotide sequence represented by SEQ ID NO: 81373.

The probes having the nucleotide sequence represented by SEQ ID NOS: 150304, 150305, 150306, 150307, 150308, 150309, 150310, 150311, 150312, 150313, 150314 were selected from the ORFs having the nucleotide sequence represented by SEQ ID NO: 81374.

The probes having the nucleotide sequence represented by SEQ ID NOS: 150315, 150316, 150317, 150318, 150319, 150320, 150321, 150322, 150323, 150324, 150325 were selected from the ORFs having the nucleotide sequence represented by SEQ ID NO: 81375.

The probes having the nucleotide sequence represented by SEQ ID NOS: 150326, 150327, 150328, 150329, 150330, 150331, 150332, 150333, 150334, 150335, 150336 were selected from the ORFs having the nucleotide sequence represented by SEQ ID NO: 81376.

The probes having the nucleotide sequence represented by SEQ ID NOS: 150337, 150338, 150339, 150340, 150341, 150342, 150343, 150344, 150345, 150346, 150347 were selected from the ORFs having the nucleotide sequence represented by SEQ ID NO: 81377.

The probes having the nucleotide sequence represented by SEQ ID NOS: 150348, 150349, 150350, 150351, 150352, 150353, 150354, 150355, 150356, 150357, 150358 were selected from the ORFs having the nucleotide sequence represented by SEQ ID NO: 81378.

The probes having the nucleotide sequence represented by SEQ ID NOS: 150359, 150360, 150361, 150362, 150363, 150364, 150365, 150366, 150367, 150368, 150369 were selected from the ORFs having the nucleotide sequence represented by SEQ ID NO: 81379.

The probes having the nucleotide sequence represented by SEQ ID NOS: 150370, 150371, 150372, 150373, 150374, 150375, 150376, 150377, 150378, 150379, 150380 were selected from the ORFs having the nucleotide sequence represented by SEQ ID NO: 81380.

The probes having the nucleotide sequence represented by SEQ ID NOS: 150381, 150382, 150383, 150384, 150385, 150386, 150387, 150388, 150389, 150390, 150391 were selected from the ORFs having the nucleotide sequence represented by SEQ ID NO: 81381.

The probes having the nucleotide sequence represented by SEQ ID NOS: 150392, 150393, 150394, 150395, 150396, 150397, 150398, 150399, 150400, 150401, 150402 were selected from the ORFs having the nucleotide sequence represented by SEQ ID NO: 81382.

The probes having the nucleotide sequence represented by SEQ ID NOS: 150403, 150404, 150405, 150406, 150407, 150408, 150409, 150410, 150411, 150412, 150413 were selected from the ORFs having the nucleotide sequence represented by SEQ ID NO: 81383.

The probes having the nucleotide sequence represented by SEQ ID NOS: 150414, 150415, 150416, 150417, 150418, 150419, 150420, 150421, 150422, 150423, 150424 were selected from the ORFs having the nucleotide sequence represented by SEQ ID NO: 81384.

The probes having the nucleotide sequence represented by SEQ ID NOS: 150425, 150426, 150427, 150428, 150429, 150430, 150431, 150432, 150433, 150434, 150435 were selected from the ORFs having the nucleotide sequence represented by SEQ ID NO: 81385.

The probes having the nucleotide sequence represented by SEQ ID NOS: 150436, 150437, 150438, 150439, 150440, 150441, 150442, 150443, 150444, 150445, 150446 were selected from the ORFs having the nucleotide sequence represented by SEQ ID NO: 81386.

The probes having the nucleotide sequence represented by SEQ ID NOS: 150447, 150448, 150449, 150450, 150451, 150452, 150453, 150454, 150455, 150456, 150457 were selected from the ORFs having the nucleotide sequence represented by SEQ ID NO: 81387.

The probes having the nucleotide sequence represented by SEQ ID NOS: 150458, 150459, 150460, 150461, 150462, 150463, 150464, 150465, 150466, 150467, 150468 were selected from the ORFs having the nucleotide sequence represented by SEQ ID NO: 81388.

The probes having the nucleotide sequence represented by SEQ ID NOS: 150469, 150470, 150471, 150472, 150473, 150474, 150475, 150476, 150477, 150478, 150479 were selected from the ORFs having the nucleotide sequence represented by SEQ ID NO: 81389.

The probes having the nucleotide sequence represented by SEQ ID NOS: 150480, 150481, 150482, 150483, 150484, 150485, 150486, 150487, 150488, 150489, 150490 were selected from the ORFs having the nucleotide sequence represented by SEQ ID NO: 81390.

The probes having the nucleotide sequence represented by SEQ ID NOS: 150491, 150492, 150493, 150494, 150495, 150496, 150497, 150498, 150499, 150500, 150501 were selected from the ORFs having the nucleotide sequence represented by SEQ ID NO: 81391.

The probes having the nucleotide sequence represented by SEQ ID NOS: 150502, 150503, 150504, 150505, 150506, 150507, 150508, 150509, 150510, 150511, 150512 were selected from the ORFs having the nucleotide sequence represented by SEQ ID NO: 81392.

The probes having the nucleotide sequence represented by SEQ ID NOS: 150513, 150514, 150515, 150516, 150517, 150518, 150519, 150520, 150521, 150522, 150523 were selected from the ORFs having the nucleotide sequence represented by SEQ ID NO: 81393.

The probes having the nucleotide sequence represented by SEQ ID NOS: 150524, 150525, 150526, 150527, 150528, 150529, 150530, 150531, 150532, 150533, 150534 were selected from the ORFs having the nucleotide sequence represented by SEQ ID NO: 81394.

The probes having the nucleotide sequence represented by SEQ ID NOS: 150535, 150536, 150537, 150538, 150539, 150540, 150541, 150542, 150543, 150544, 150545 were selected from the ORFs having the nucleotide sequence represented by SEQ ID NO: 81395.

The probes having the nucleotide sequence represented by SEQ ID NOS: 150546, 150547, 150548, 150549, 150550, 150551, 150552, 150553, 150554, 150555, 150556 were selected from the ORFs having the nucleotide sequence represented by SEQ ID NO: 81396.

The probes having the nucleotide sequence represented by SEQ ID NOS: 150557, 150558, 150559, 150560, 150561, 150562, 150563, 150564, 150565, 150566, 150567 were selected from the ORFs having the nucleotide sequence represented by SEQ ID NO: 81397.

The probes having the nucleotide sequence represented by SEQ ID NOS: 150568, 150569, 150570, 150571, 150572, 150573, 150574, 150575, 150576, 150577, 150578 were selected from the ORFs having the nucleotide sequence represented by SEQ ID NO: 81398.

The probes having the nucleotide sequence represented by SEQ ID NOS: 150579, 150580, 150581, 150582, 150583, 150584, 150585, 150586, 150587, 150588, 150589 were selected from the ORFs having the nucleotide sequence represented by SEQ ID NO: 81399.

The probes having the nucleotide sequence represented by SEQ ID NOS: 150590, 150591, 150592, 150593, 150594, 150595, 150596, 150597, 150598, 150599, 150600 were selected from the ORFs having the nucleotide sequence represented by SEQ ID NO: 81400.

The probes having the nucleotide sequence represented by SEQ ID NOS: 150601, 150602, 150603, 150604, 150605, 150606, 150607, 150608, 150609, 150610, 150611 were selected from the ORFs having the nucleotide sequence represented by SEQ ID NO: 81401.

The probes having the nucleotide sequence represented by SEQ ID NOS: 150612, 150613, 150614, 150615, 150616, 150617, 150618, 150619, 150620, 150621, 150622 were selected from the ORFs having the nucleotide sequence represented by SEQ ID NO: 81402.

The probes having the nucleotide sequence represented by SEQ ID NOS: 150623, 150624, 150625, 150626, 150627, 150628, 150629, 150630, 150631, 150632, 150633 were selected from the ORFs having the nucleotide sequence represented by SEQ ID NO: 81403.

The probes having the nucleotide sequence represented by SEQ ID NOS: 150634, 150635, 150636, 150637, 150638, 150639, 150640, 150641, 150642, 150643, 150644 were selected from the ORFs having the nucleotide sequence represented by SEQ ID NO: 81404.

The probes having the nucleotide sequence represented by SEQ ID NOS: 150645, 150646, 150647, 150648, 150649, 150650, 150651, 150652, 150653, 150654, 150655 were selected from the ORFs having the nucleotide sequence represented by SEQ ID NO: 81405.

The probes having the nucleotide sequence represented by SEQ ID NOS: 150656, 150657, 150658, 150659, 150660, 150661, 150662, 150663, 150664, 150665, 150666 were selected from the ORFs having the nucleotide sequence represented by SEQ ID NO: 81406.

The probes having the nucleotide sequence represented by SEQ ID NOS: 150667, 150668, 150669, 150670, 150671, 150672, 150673, 150674, 150675, 150676, 150677 were selected from the ORFs having the nucleotide sequence represented by SEQ ID NO: 81407.

The probes having the nucleotide sequence represented by SEQ ID NOS: 150678, 150679, 150680, 150681, 150682, 150683, 150684, 150685, 150686, 150687, 150688 were selected from the ORFs having the nucleotide sequence represented by SEQ ID NO: 81408.

The probes having the nucleotide sequence represented by SEQ ID NOS: 150689, 150690, 150691, 150692, 150693, 150694, 150695, 150696, 150697, 150698, 150699, 150700, 150701, 150702, 150703, 150704, 150705, 150706, 150707, 150708, 150709, 150710 were selected from the ORFs having the nucleotide sequence represented by SEQ ID NO: 81409.

The probes having the nucleotide sequence represented by SEQ ID NOS: 150711, 150712, 150713, 150714, 150715, 150716, 150717, 150718, 150719, 150720, 150721 were selected from the ORFs having the nucleotide sequence represented by SEQ ID NO: 81410.

The probes having the nucleotide sequence represented by SEQ ID NOS: 150722, 150723, 150724, 150725, 150726, 150727, 150728, 150729, 150730, 150731, 150732 were selected from the ORFs having the nucleotide sequence represented by SEQ ID NO: 81411.

The probes having the nucleotide sequence represented by SEQ ID NOS: 150733, 150734, 150735, 150736, 150737, 150738, 150739, 150740, 150741, 150742, 150743 were selected from the ORFs having the nucleotide sequence represented by SEQ ID NO: 81412.

The probes having the nucleotide sequence represented by SEQ ID NOS: 150744, 150745, 150746, 150747, 150748, 150749, 150750, 150751, 150752, 150753, 150754 were selected from the ORFs having the nucleotide sequence represented by SEQ ID NO: 81413.

The probes having the nucleotide sequence represented by SEQ ID NOS: 150755, 150756, 150757, 150758, 150759, 150760, 150761, 150762, 150763, 150764, 150765 were selected from the ORFs having the nucleotide sequence represented by SEQ ID NO: 81414.

The probes having the nucleotide sequence represented by SEQ ID NOS: 150766, 150767, 150768, 150769, 150770, 150771, 150772, 150773, 150774, 150775, 150776 were selected from the ORFs having the nucleotide sequence represented by SEQ ID NO: 81415.

The probes having the nucleotide sequence represented by SEQ ID NOS: 150777, 150778, 150779, 150780, 150781, 150782, 150783, 150784, 150785, 150786, 150787 were selected from the ORFs having the nucleotide sequence represented by SEQ ID NO: 81416.

The probes having the nucleotide sequence represented by SEQ ID NOS: 150788, 150789, 150790, 150791, 150792, 150793, 150794, 150795, 150796, 150797, 150798, 150799, 150800, 150801, 150802, 150803, 150804, 150805, 150806, 150807, 150808, 150809 were selected from the ORFs having the nucleotide sequence represented by SEQ ID NO: 81417.

The probes having the nucleotide sequence represented by SEQ ID NOS: 150810, 150811, 150812, 150813, 150814, 150815, 150816, 150817, 150818, 150819, 150820 were selected from the ORFs having the nucleotide sequence represented by SEQ ID NO: 81418.

The probes having the nucleotide sequence represented by SEQ ID NOS: 150821, 150822, 150823, 150824, 150825, 150826, 150827, 150828, 150829, 150830, 150831 were selected from the ORFs having the nucleotide sequence represented by SEQ ID NO: 81419.

The probes having the nucleotide sequence represented by SEQ ID NOS: 150832, 150833, 150834, 150835, 150836, 150837, 150838, 150839, 150840, 150841, 150842 were selected from the ORFs having the nucleotide sequence represented by SEQ ID NO: 81420.

The probes having the nucleotide sequence represented by SEQ ID NOS: 150843, 150844, 150845, 150846, 150847, 150848, 150849, 150850, 150851, 150852, 150853 were selected from the ORFs having the nucleotide sequence represented by SEQ ID NO: 81421.

The probes having the nucleotide sequence represented by SEQ ID NOS: 150854, 150855, 150856, 150857, 150858, 150859, 150860, 150861, 150862, 150863, 150864 were selected from the ORFs having the nucleotide sequence represented by SEQ ID NO: 81422.

The probes having the nucleotide sequence represented by SEQ ID NOS: 150865, 150866, 150867, 150868, 150869, 150870, 150871, 150872, 150873, 150874, 150875 were selected from the ORFs having the nucleotide sequence represented by SEQ ID NO: 81423.

The probes having the nucleotide sequence represented by SEQ ID NOS: 150876, 150877, 150878, 150879, 150880, 150881, 150882, 150883, 150884, 150885, 150886 were selected from the ORFs having the nucleotide sequence represented by SEQ ID NO: 81424.

The probes having the nucleotide sequence represented by SEQ ID NOS: 150887, 150888, 150889, 150890, 150891, 150892, 150893, 150894, 150895, 150896, 150897 were selected from the ORFs having the nucleotide sequence represented by SEQ ID NO: 81425.

The probes having the nucleotide sequence represented by SEQ ID NOS: 150898, 150899, 150900, 150901, 150902, 150903, 150904, 150905, 150906, 150907, 150908 were selected from the ORFs having the nucleotide sequence represented by SEQ ID NO: 81426.

The probes having the nucleotide sequence represented by SEQ ID NOS: 150909, 150910, 150911, 150912, 150913, 150914, 150915, 150916, 150917, 150918, 150919 were selected from the ORFs having the nucleotide sequence represented by SEQ ID NO: 81427.

The probes having the nucleotide sequence represented by SEQ ID NOS: 150920, 150921, 150922, 150923, 150924, 150925, 150926, 150927, 150928, 150929, 150930 were selected from the ORFs having the nucleotide sequence represented by SEQ ID NO: 81428.

The probes having the nucleotide sequence represented by SEQ ID NOS: 150931, 150932, 150933, 150934, 150935, 150936, 150937, 150938, 150939, 150940, 150941 were selected from the ORFs having the nucleotide sequence represented by SEQ ID NO: 81429.

The probes having the nucleotide sequence represented by SEQ ID NOS: 150942, 150943, 150944, 150945, 150946, 150947, 150948, 150949, 150950, 150951, 150952, 150953, 150954, 150955, 150956, 150957, 150958, 150959, 150960, 150961, 150962, 150963 were selected from the ORFs having the nucleotide sequence represented by SEQ ID NO: 81430.

The probes having the nucleotide sequence represented by SEQ ID NOS: 150964, 150965, 150966, 150967, 150968, 150969, 150970, 150971, 150972, 150973, 150974 were selected from the ORFs having the nucleotide sequence represented by SEQ ID NO: 81431.

The probes having the nucleotide sequence represented by SEQ ID NOS: 150975, 150976, 150977, 150978, 150979, 150980, 150981, 150982, 150983, 150984, 150985 were selected from the ORFs having the nucleotide sequence represented by SEQ ID NO: 81432.

The probes having the nucleotide sequence represented by SEQ ID NOS: 150986, 150987, 150988, 150989, 150990, 150991, 150992, 150993, 150994, 150995, 150996 were selected from the ORFs having the nucleotide sequence represented by SEQ ID NO: 81433.

The probes having the nucleotide sequence represented by SEQ ID NOS: 150997, 150998, 150999, 151000, 151001, 151002, 151003, 151004, 151005, 151006, 151007 were selected from the ORFs having the nucleotide sequence represented by SEQ ID NO: 81434.

The probes having the nucleotide sequence represented by SEQ ID NOS: 151008, 151009, 151010, 151011, 151012, 151013, 151014, 151015, 151016, 151017, 151018 were selected from the ORFs having the nucleotide sequence represented by SEQ ID NO: 81435.

The probes having the nucleotide sequence represented by SEQ ID NOS: 151019, 151020, 151021, 151022, 151023, 151024, 151025, 151026, 151027, 151028, 151029 were selected from the ORFs having the nucleotide sequence represented by SEQ ID NO: 81436.

The probes having the nucleotide sequence represented by SEQ ID NOS: 151030, 151031, 151032, 151033, 151034, 151035, 151036, 151037, 151038, 151039, 151040 were selected from the ORFs having the nucleotide sequence represented by SEQ ID NO: 81437.

The probes having the nucleotide sequence represented by SEQ ID NOS: 151041, 151042, 151043, 151044, 151045, 151046, 151047, 151048, 151049, 151050, 151051 were selected from the ORFs having the nucleotide sequence represented by SEQ ID NO: 81438.

The probes having the nucleotide sequence represented by SEQ ID NOS: 151052, 151053, 151054, 151055, 151056, 151057, 151058, 151059, 151060, 151061, 151062 were selected from the ORFs having the nucleotide sequence represented by SEQ ID NO: 81439.

The probes having the nucleotide sequence represented by SEQ ID NOS: 151063, 151064, 151065, 151066, 151067, 151068, 151069, 151070, 151071, 151072, 151073 were selected from the ORFs having the nucleotide sequence represented by SEQ ID NO: 81440.

The probes having the nucleotide sequence represented by SEQ ID NOS: 151074, 151075, 151076, 151077, 151078, 151079, 151080, 151081, 151082, 151083, 151084 were selected from the ORFs having the nucleotide sequence represented by SEQ ID NO: 81441.

The probes having the nucleotide sequence represented by SEQ ID NOS: 151085, 151086, 151087, 151088, 151089, 151090, 151091, 151092, 151093, 151094, 151095 were selected from the ORFs having the nucleotide sequence represented by SEQ ID NO: 81442.

The probes having the nucleotide sequence represented by SEQ ID NOS: 151096, 151097, 151098, 151099, 151100, 151101, 151102, 151103, 151104, 151105, 151106 were selected from the ORFs having the nucleotide sequence represented by SEQ ID NO: 81443.

The probes having the nucleotide sequence represented by SEQ ID NOS: 151107, 151108, 151109, 151110, 151111, 151112, 151113, 151114, 151115, 151116, 151117 were selected from the ORFs having the nucleotide sequence represented by SEQ ID NO: 81444.

The probes having the nucleotide sequence represented by SEQ ID NOS: 151118, 151119, 151120, 151121, 151122, 151123, 151124, 151125, 151126, 151127, 151128 were selected from the ORFs having the nucleotide sequence represented by SEQ ID NO: 81445.

The probes having the nucleotide sequence represented by SEQ ID NOS: 151129, 151130, 151131, 151132, 151133, 151134, 151135, 151136, 151137, 151138, 151139 were selected from the ORFs having the nucleotide sequence represented by SEQ ID NO: 81446.

The probes having the nucleotide sequence represented by SEQ ID NOS: 151140, 151141, 151142, 151143, 151144, 151145, 151146, 151147, 151148, 151149, 151150 were selected from the ORFs having the nucleotide sequence represented by SEQ ID NO: 81447.

The probes having the nucleotide sequence represented by SEQ ID NOS: 151151, 151152, 151153, 151154, 151155, 151156, 151157, 151158, 151159, 151160, 151161 were selected from the ORFs having the nucleotide sequence represented by SEQ ID NO: 81448.

The probes having the nucleotide sequence represented by SEQ ID NOS: 151162, 151163, 151164, 151165, 151166, 151167, 151168, 151169, 151170, 151171, 151172 were selected from the ORFs having the nucleotide sequence represented by SEQ ID NO: 81449.

The probes having the nucleotide sequence represented by SEQ ID NOS: 151173, 151174, 151175, 151176, 151177, 151178, 151179, 151180, 151181, 151182, 151183 were selected from the ORFs having the nucleotide sequence represented by SEQ ID NO: 81450.

The probes having the nucleotide sequence represented by SEQ ID NOS: 151184, 151185, 151186, 151187, 151188, 151189, 151190, 151191, 151192, 151193, 151194 were selected from the ORFs having the nucleotide sequence represented by SEQ ID NO: 81451.

The probes having the nucleotide sequence represented by SEQ ID NOS: 151195, 151196, 151197, 151198, 151199, 151200, 151201, 151202, 151203, 151204, 151205 were selected from the ORFs having the nucleotide sequence represented by SEQ ID NO: 81452.

The probes having the nucleotide sequence represented by SEQ ID NOS: 151206, 151207, 151208, 151209, 151210, 151211, 151212, 151213, 151214, 151215, 151216 were selected from the ORFs having the nucleotide sequence represented by SEQ ID NO: 81453.

The probes having the nucleotide sequence represented by SEQ ID NOS: 151217, 151218, 151219, 151220, 151221, 151222, 151223, 151224, 151225, 151226, 151227 were selected from the ORFs having the nucleotide sequence represented by SEQ ID NO: 81454.

The probes having the nucleotide sequence represented by SEQ ID NOS: 151228, 151229, 151230, 151231, 151232, 151233, 151234, 151235, 151236, 151237, 151238 were selected from the ORFs having the nucleotide sequence represented by SEQ ID NO: 81455.

The probes having the nucleotide sequence represented by SEQ ID NOS: 151239, 151240, 151241, 151242, 151243, 151244, 151245, 151246, 151247, 151248, 151249 were selected from the ORFs having the nucleotide sequence represented by SEQ ID NO: 81456.

The probes having the nucleotide sequence represented by SEQ ID NOS: 151250, 151251, 151252, 151253, 151254, 151255, 151256, 151257, 151258, 151259, 151260 were selected from the ORFs having the nucleotide sequence represented by SEQ ID NO: 81457.

The probes having the nucleotide sequence represented by SEQ ID NOS: 151261, 151262, 151263, 151264, 151265, 151266, 151267, 151268, 151269, 151270, 151271 were selected from the ORFs having the nucleotide sequence represented by SEQ ID NO: 81458.

The probes having the nucleotide sequence represented by SEQ ID NOS: 151272, 151273, 151274, 151275, 151276, 151277, 151278, 151279, 151280, 151281, 151282 were selected from the ORFs having the nucleotide sequence represented by SEQ ID NO: 81459.

The probes having the nucleotide sequence represented by SEQ ID NOS: 151283, 151284, 151285, 151286, 151287, 151288, 151289, 151290, 151291, 151292, 151293 were selected from the ORFs having the nucleotide sequence represented by SEQ ID NO: 81460.

The probes having the nucleotide sequence represented by SEQ ID NOS: 151294, 151295, 151296, 151297, 151298, 151299, 151300, 151301, 151302, 151303, 151304 were selected from the ORFs having the nucleotide sequence represented by SEQ ID NO: 81461.

The probes having the nucleotide sequence represented by SEQ ID NOS: 151305, 151306, 151307, 151308, 151309, 151310, 151311, 151312, 151313, 151314, 151315 were selected from the ORFs having the nucleotide sequence represented by SEQ ID NO: 81462.

The probes having the nucleotide sequence represented by SEQ ID NOS: 151316, 151317, 151318, 151319, 151320, 151321, 151322, 151323, 151324, 151325, 151326 were selected from the ORFs having the nucleotide sequence represented by SEQ ID NO: 81463.

The probes having the nucleotide sequence represented by SEQ ID NOS: 151327, 151328, 151329, 151330, 151331, 151332, 151333, 151334, 151335, 151336, 151337 were selected from the ORFs having the nucleotide sequence represented by SEQ ID NO: 81464.

The probes having the nucleotide sequence represented by SEQ ID NOS: 151338, 151339, 151340, 151341, 151342, 151343, 151344, 151345, 151346, 151347, 151348 were selected from the ORFs having the nucleotide sequence represented by SEQ ID NO: 81465.

The probes having the nucleotide sequence represented by SEQ ID NOS: 151349, 151350, 151351, 151352, 151353, 151354, 151355, 151356, 151357, 151358, 151359 were selected from the ORFs having the nucleotide sequence represented by SEQ ID NO: 81466.

The probes having the nucleotide sequence represented by SEQ ID NOS: 151360, 151361, 151362, 151363, 151364, 151365, 151366, 151367, 151368, 151369, 151370 were selected from the ORFs having the nucleotide sequence represented by SEQ ID NO: 81467.

The probes having the nucleotide sequence represented by SEQ ID NOS: 151371, 151372, 151373, 151374, 151375, 151376, 151377, 151378, 151379, 151380, 151381 were selected from the ORFs having the nucleotide sequence represented by SEQ ID NO: 81468.

The probes having the nucleotide sequence represented by SEQ ID NOS: 151382, 151383, 151384, 151385, 151386, 151387, 151388, 151389, 151390, 151391, 151392 were selected from the ORFs having the nucleotide sequence represented by SEQ ID NO: 81469.

The probes having the nucleotide sequence represented by SEQ ID NOS: 151393, 151394, 151395, 151396, 151397, 151398, 151399, 151400, 151401, 151402, 151403 were selected from the ORFs having the nucleotide sequence represented by SEQ ID NO: 81470.

The probes having the nucleotide sequence represented by SEQ ID NOS: 151404, 151405, 151406, 151407, 151408, 151409, 151410, 151411, 151412, 151413, 151414 were selected from the ORFs having the nucleotide sequence represented by SEQ ID NO: 81471.

The probes having the nucleotide sequence represented by SEQ ID NOS: 151415, 151416, 151417, 151418, 151419, 151420, 151421, 151422, 151423, 151424, 151425 were selected from the ORFs having the nucleotide sequence represented by SEQ ID NO: 81472.

The probes having the nucleotide sequence represented by SEQ ID NOS: 151426, 151427, 151428, 151429, 151430, 151431, 151432, 151433, 151434, 151435, 151436 were selected from the ORFs having the nucleotide sequence represented by SEQ ID NO: 81473.

The probes having the nucleotide sequence represented by SEQ ID NOS: 151437, 151438, 151439, 151440, 151441, 151442, 151443, 151444, 151445, 151446, 151447 were selected from the ORFs having the nucleotide sequence represented by SEQ ID NO: 81474.

The probes having the nucleotide sequence represented by SEQ ID NOS: 151448, 151449, 151450, 151451, 151452, 151453, 151454, 151455, 151456, 151457, 151458 were selected from the ORFs having the nucleotide sequence represented by SEQ ID NO: 81475.

The probes having the nucleotide sequence represented by SEQ ID NOS: 151459, 151460, 151461, 151462, 151463, 151464, 151465, 151466, 151467, 151468, 151469, 151470, 151471, 151472, 151473, 151474, 151475, 151476, 151477, 151478, 151479, 151480 were selected from the ORFs having the nucleotide sequence represented by SEQ ID NO: 81476.

The probes having the nucleotide sequence represented by SEQ ID NOS: 151481, 151482, 151483, 151484, 151485, 151486, 151487, 151488, 151489, 151490, 151491 were selected from the ORFs having the nucleotide sequence represented by SEQ ID NO: 81477.

The probes having the nucleotide sequence represented by SEQ ID NOS: 151492, 151493, 151494, 151495, 151496, 151497, 151498, 151499, 151500, 151501, 151502 were selected from the ORFs having the nucleotide sequence represented by SEQ ID NO: 81478.

The probes having the nucleotide sequence represented by SEQ ID NOS: 151503, 151504, 151505, 151506, 151507, 151508, 151509, 151510, 151511, 151512, 151513 were selected from the ORFs having the nucleotide sequence represented by SEQ ID NO: 81479.

The probes having the nucleotide sequence represented by SEQ ID NOS: 151514, 151515, 151516, 151517, 151518, 151519, 151520, 151521, 151522, 151523, 151524 were selected from the ORFs having the nucleotide sequence represented by SEQ ID NO: 81480.

The probes having the nucleotide sequence represented by SEQ ID NOS: 151525, 151526, 151527, 151528, 151529, 151530, 151531, 151532, 151533, 151534, 151535 were selected from the ORFs having the nucleotide sequence represented by SEQ ID NO: 81481.

The probes having the nucleotide sequence represented by SEQ ID NOS: 151536, 151537, 151538, 151539, 151540, 151541, 151542, 151543, 151544, 151545, 151546 were selected from the ORFs having the nucleotide sequence represented by SEQ ID NO: 81482.

The probes having the nucleotide sequence represented by SEQ ID NOS: 151547, 151548, 151549, 151550, 151551, 151552, 151553, 151554, 151555, 151556, 151557 were selected from the ORFs having the nucleotide sequence represented by SEQ ID NO: 81483.

The probes having the nucleotide sequence represented by SEQ ID NOS: 151558, 151559, 151560, 151561, 151562, 151563, 151564, 151565, 151566, 151567, 151568 were selected from the ORFs having the nucleotide sequence represented by SEQ ID NO: 81484.

The probes having the nucleotide sequence represented by SEQ ID NOS: 151569, 151570, 151571, 151572, 151573, 151574, 151575, 151576, 151577, 151578, 151579 were selected from the ORFs having the nucleotide sequence represented by SEQ ID NO: 81485.

The probes having the nucleotide sequence represented by SEQ ID NOS: 151580, 151581, 151582, 151583, 151584, 151585, 151586, 151587, 151588, 151589, 151590 were selected from the ORFs having the nucleotide sequence represented by SEQ ID NO: 81486.

The probes having the nucleotide sequence represented by SEQ ID NOS: 151591, 151592, 151593, 151594, 151595, 151596, 151597, 151598, 151599, 151600, 151601 were selected from the ORFs having the nucleotide sequence represented by SEQ ID NO: 81487.

The probes having the nucleotide sequence represented by SEQ ID NOS: 151602, 151603, 151604, 151605, 151606, 151607, 151608, 151609, 151610, 151611, 151612 were selected from the ORFs having the nucleotide sequence represented by SEQ ID NO: 81488.

The probes having the nucleotide sequence represented by SEQ ID NOS: 151613, 151614, 151615, 151616, 151617, 151618, 151619, 151620, 151621, 151622, 151623 were selected from the ORFs having the nucleotide sequence represented by SEQ ID NO: 81489.

The probes having the nucleotide sequence represented by SEQ ID NOS: 151624, 151625, 151626, 151627, 151628, 151629, 151630, 151631, 151632, 151633, 151634 were selected from the ORFs having the nucleotide sequence represented by SEQ ID NO: 81490.

The probes having the nucleotide sequence represented by SEQ ID NOS: 151635, 151636, 151637, 151638, 151639, 151640, 151641, 151642, 151643, 151644, 151645 were selected from the ORFs having the nucleotide sequence represented by SEQ ID NO: 81491.

The probes having the nucleotide sequence represented by SEQ ID NOS: 151646, 151647, 151648, 151649, 151650, 151651, 151652, 151653, 151654, 151655, 151656 were selected from the ORFs having the nucleotide sequence represented by SEQ ID NO: 81492.

The probes having the nucleotide sequence represented by SEQ ID NOS: 151657, 151658, 151659, 151660, 151661, 151662, 151663, 151664, 151665, 151666, 151667 were selected from the ORFs having the nucleotide sequence represented by SEQ ID NO: 81493.

The probes having the nucleotide sequence represented by SEQ ID NOS: 151668, 151669, 151670, 151671, 151672, 151673, 151674, 151675, 151676, 151677, 151678 were selected from the ORFs having the nucleotide sequence represented by SEQ ID NO: 81494.

The probes having the nucleotide sequence represented by SEQ ID NOS: 151679, 151680, 151681, 151682, 151683, 151684, 151685, 151686, 151687, 151688, 151689 were selected from the ORFs having the nucleotide sequence represented by SEQ ID NO: 81495.

The probes having the nucleotide sequence represented by SEQ ID NOS: 151690, 151691, 151692, 151693, 151694, 151695, 151696, 151697, 151698, 151699, 151700 were selected from the ORFs having the nucleotide sequence represented by SEQ ID NO: 81496.

The probes having the nucleotide sequence represented by SEQ ID NOS: 151701, 151702, 151703, 151704, 151705, 151706, 151707, 151708, 151709, 151710, 151711 were selected from the ORFs having the nucleotide sequence represented by SEQ ID NO: 81497.

The probes having the nucleotide sequence represented by SEQ ID NOS: 151712, 151713, 151714, 151715, 151716, 151717, 151718, 151719, 151720, 151721, 151722 were selected from the ORFs having the nucleotide sequence represented by SEQ ID NO: 81498.

The probes having the nucleotide sequence represented by SEQ ID NOS: 151723, 151724, 151725, 151726, 151727, 151728, 151729, 151730, 151731, 151732, 151733 were selected from the ORFs having the nucleotide sequence represented by SEQ ID NO: 81499.

The probes having the nucleotide sequence represented by SEQ ID NOS: 151734, 151735, 151736, 151737, 151738, 151739, 151740, 151741, 151742, 151743, 151744 were selected from the ORFs having the nucleotide sequence represented by SEQ ID NO: 81500.

The probes having the nucleotide sequence represented by SEQ ID NOS: 151745, 151746, 151747, 151748, 151749, 151750, 151751, 151752, 151753, 151754, 151755 were selected from the ORFs having the nucleotide sequence represented by SEQ ID NO: 81501.

The probes having the nucleotide sequence represented by SEQ ID NOS: 151756, 151757, 151758, 151759, 151760, 151761, 151762, 151763, 151764, 151765, 151766 were selected from the ORFs having the nucleotide sequence represented by SEQ ID NO: 81502.

The probes having the nucleotide sequence represented by SEQ ID NOS: 151767, 151768, 151769, 151770, 151771, 151772, 151773, 151774, 151775, 151776, 151777 were selected from the ORFs having the nucleotide sequence represented by SEQ ID NO: 81503.

The probes having the nucleotide sequence represented by SEQ ID NOS: 151778, 151779, 151780, 151781, 151782, 151783, 151784, 151785, 151786, 151787, 151788 were selected from the ORFs having the nucleotide sequence represented by SEQ ID NO: 81504.

The probes having the nucleotide sequence represented by SEQ ID NOS: 151789, 151790, 151791, 151792, 151793, 151794, 151795, 151796, 151797, 151798, 151799 were selected from the ORFs having the nucleotide sequence represented by SEQ ID NO: 81505.

The probes having the nucleotide sequence represented by SEQ ID NOS: 151800, 151801, 151802, 151803, 151804, 151805, 151806, 151807, 151808, 151809, 151810 were selected from the ORFs having the nucleotide sequence represented by SEQ ID NO: 81506.

The probes having the nucleotide sequence represented by SEQ ID NOS: 151811, 151812, 151813, 151814, 151815, 151816, 151817, 151818, 151819, 151820, 151821 were selected from the ORFs having the nucleotide sequence represented by SEQ ID NO: 81507.

The probes having the nucleotide sequence represented by SEQ ID NOS: 151822, 151823, 151824, 151825, 151826, 151827, 151828, 151829, 151830, 151831, 151832 were selected from the ORFs having the nucleotide sequence represented by SEQ ID NO: 81508.

The probes having the nucleotide sequence represented by SEQ ID NOS: 151833, 151834, 151835, 151836, 151837, 151838, 151839, 151840, 151841, 151842, 151843 were selected from the ORFs having the nucleotide sequence represented by SEQ ID NO: 81509.

The probes having the nucleotide sequence represented by SEQ ID NOS: 151844, 151845, 151846, 151847, 151848, 151849, 151850, 151851, 151852, 151853, 151854 were selected from the ORFs having the nucleotide sequence represented by SEQ ID NO: 81510.

The probes having the nucleotide sequence represented by SEQ ID NOS: 151855, 151856, 151857, 151858, 151859, 151860, 151861, 151862, 151863, 151864, 151865 were selected from the ORFs having the nucleotide sequence represented by SEQ ID NO: 81511.

The probes having the nucleotide sequence represented by SEQ ID NOS: 151866, 151867, 151868, 151869, 151870, 151871, 151872, 151873, 151874, 151875, 151876 were selected from the ORFs having the nucleotide sequence represented by SEQ ID NO: 81512.

The probes having the nucleotide sequence represented by SEQ ID NOS: 151877, 151878, 151879, 151880, 151881, 151882, 151883, 151884, 151885, 151886, 151887 were selected from the ORFs having the nucleotide sequence represented by SEQ ID NO: 81513.

The probes having the nucleotide sequence represented by SEQ ID NOS: 151888, 151889, 151890, 151891, 151892, 151893, 151894, 151895, 151896, 151897, 151898 were selected from the ORFs having the nucleotide sequence represented by SEQ ID NO: 81514.

The probes having the nucleotide sequence represented by SEQ ID NOS: 151899, 151900, 151901, 151902, 151903, 151904, 151905, 151906, 151907, 151908, 151909 were selected from the ORFs having the nucleotide sequence represented by SEQ ID NO: 81515.

The probes having the nucleotide sequence represented by SEQ ID NOS: 151910, 151911, 151912, 151913, 151914, 151915, 151916, 151917, 151918, 151919, 151920 were selected from the ORFs having the nucleotide sequence represented by SEQ ID NO: 81516.

The probes having the nucleotide sequence represented by SEQ ID NOS: 151921, 151922, 151923, 151924, 151925, 151926, 151927, 151928, 151929, 151930, 151931 were selected from the ORFs having the nucleotide sequence represented by SEQ ID NO: 81517.

The probes having the nucleotide sequence represented by SEQ ID NOS: 151932, 151933, 151934, 151935, 151936, 151937, 151938, 151939, 151940, 151941, 151942, 151943, 151944, 151945, 151946, 151947, 151948, 151949, 151950, 151951, 151952, 151953 were selected from the ORFs having the nucleotide sequence represented by SEQ ID NO: 81518.

The probes having the nucleotide sequence represented by SEQ ID NOS: 151954, 151955, 151956, 151957, 151958, 151959, 151960, 151961, 151962, 151963, 151964 were selected from the ORFs having the nucleotide sequence represented by SEQ ID NO: 81519.

The probes having the nucleotide sequence represented by SEQ ID NOS: 151965, 151966, 151967, 151968, 151969, 151970, 151971, 151972, 151973, 151974, 151975 were selected from the ORFs having the nucleotide sequence represented by SEQ ID NO: 81520.

The probes having the nucleotide sequence represented by SEQ ID NOS: 151976, 151977, 151978, 151979, 151980, 151981, 151982, 151983, 151984, 151985, 151986 were selected from the ORFs having the nucleotide sequence represented by SEQ ID NO: 81521.

The probes having the nucleotide sequence represented by SEQ ID NOS: 151987, 151988, 151989, 151990, 151991, 151992, 151993, 151994, 151995, 151996, 151997 were selected from the ORFs having the nucleotide sequence represented by SEQ ID NO: 81522.

The probes having the nucleotide sequence represented by SEQ ID NOS: 151998, 151999, 152000, 152001, 152002, 152003, 152004, 152005, 152006, 152007, 152008 were selected from the ORFs having the nucleotide sequence represented by SEQ ID NO: 81523.

The probes having the nucleotide sequence represented by SEQ ID NOS: 152009, 152010, 152011, 152012, 152013, 152014, 152015, 152016, 152017, 152018, 152019 were selected from the ORFs having the nucleotide sequence represented by SEQ ID NO: 81524.

The probes having the nucleotide sequence represented by SEQ ID NOS: 152020, 152021, 152022, 152023, 152024, 152025, 152026, 152027, 152028, 152029, 152030 were selected from the ORFs having the nucleotide sequence represented by SEQ ID NO: 81525.

The probes having the nucleotide sequence represented by SEQ ID NOS: 152031, 152032, 152033, 152034, 152035, 152036, 152037, 152038, 152039, 152040, 152041 were selected from the ORFs having the nucleotide sequence represented by SEQ ID NO: 81526.

The probes having the nucleotide sequence represented by SEQ ID NOS: 152042, 152043, 152044, 152045, 152046, 152047, 152048, 152049, 152050, 152051, 152052 were selected from the ORFs having the nucleotide sequence represented by SEQ ID NO: 81527.

The probes having the nucleotide sequence represented by SEQ ID NOS: 152053, 152054, 152055, 152056, 152057, 152058, 152059, 152060, 152061, 152062, 152063 were selected from the ORFs having the nucleotide sequence represented by SEQ ID NO: 81528.

The probes having the nucleotide sequence represented by SEQ ID NOS: 152064, 152065, 152066, 152067, 152068, 152069, 152070, 152071, 152072, 152073, 152074 were selected from the ORFs having the nucleotide sequence represented by SEQ ID NO: 81529.

The probes having the nucleotide sequence represented by SEQ ID NOS: 152075, 152076, 152077, 152078, 152079, 152080, 152081, 152082, 152083, 152084, 152085 were selected from the ORFs having the nucleotide sequence represented by SEQ ID NO: 81530.

The probes having the nucleotide sequence represented by SEQ ID NOS: 152086, 152087, 152088, 152089, 152090, 152091, 152092, 152093, 152094, 152095, 152096 were selected from the ORFs having the nucleotide sequence represented by SEQ ID NO: 81531.

The probes having the nucleotide sequence represented by SEQ ID NOS: 152097, 152098, 152099, 152100, 152101, 152102, 152103, 152104, 152105, 152106, 152107 were selected from the ORFs having the nucleotide sequence represented by SEQ ID NO: 81532.

The probes having the nucleotide sequence represented by SEQ ID NOS: 152108, 152109, 152110, 152111, 152112, 152113, 152114, 152115, 152116, 152117, 152118 were selected from the ORFs having the nucleotide sequence represented by SEQ ID NO: 81533.

The probes having the nucleotide sequence represented by SEQ ID NOS: 152119, 152120, 152121, 152122, 152123, 152124, 152125, 152126, 152127, 152128, 152129 were selected from the ORFs having the nucleotide sequence represented by SEQ ID NO: 81534.

The probes having the nucleotide sequence represented by SEQ ID NOS: 152130, 152131, 152132, 152133, 152134, 152135, 152136, 152137, 152138, 152139, 152140 were selected from the ORFs having the nucleotide sequence represented by SEQ ID NO: 81535.

The probes having the nucleotide sequence represented by SEQ ID NOS: 152141, 152142, 152143, 152144, 152145, 152146, 152147, 152148, 152149, 152150, 152151 were selected from the ORFs having the nucleotide sequence represented by SEQ ID NO: 81536.

The probes having the nucleotide sequence represented by SEQ ID NOS: 152152, 152153, 152154, 152155, 152156, 152157, 152158, 152159, 152160, 152161, 152162 were selected from the ORFs having the nucleotide sequence represented by SEQ ID NO: 81537.

The probes having the nucleotide sequence represented by SEQ ID NOS: 152163, 152164, 152165, 152166, 152167, 152168, 152169, 152170, 152171, 152172, 152173 were selected from the ORFs having the nucleotide sequence represented by SEQ ID NO: 81538.

The probes having the nucleotide sequence represented by SEQ ID NOS: 152174, 152175, 152176, 152177, 152178, 152179, 152180, 152181, 152182, 152183, 152184 were selected from the ORFs having the nucleotide sequence represented by SEQ ID NO: 81539.

The probes having the nucleotide sequence represented by SEQ ID NOS: 152185, 152186, 152187, 152188, 152189, 152190, 152191, 152192, 152193, 152194, 152195 were selected from the ORFs having the nucleotide sequence represented by SEQ ID NO: 81540.

The probes having the nucleotide sequence represented by SEQ ID NOS: 152196, 152197, 152198, 152199, 152200, 152201, 152202, 152203, 152204, 152205, 152206 were selected from the ORFs having the nucleotide sequence represented by SEQ ID NO: 81541.

The probes having the nucleotide sequence represented by SEQ ID NOS: 152207, 152208, 152209, 152210, 152211, 152212, 152213, 152214, 152215, 152216, 152217 were selected from the ORFs having the nucleotide sequence represented by SEQ ID NO: 81542.

The probes having the nucleotide sequence represented by SEQ ID NOS: 152218, 152219, 152220, 152221, 152222, 152223, 152224, 152225, 152226, 152227, 152228 were selected from the ORFs having the nucleotide sequence represented by SEQ ID NO: 81543.

The probes having the nucleotide sequence represented by SEQ ID NOS: 152229, 152230, 152231, 152232, 152233, 152234, 152235, 152236, 152237, 152238, 152239 were selected from the ORFs having the nucleotide sequence represented by SEQ ID NO: 81544.

The probes having the nucleotide sequence represented by SEQ ID NOS: 152240, 152241, 152242, 152243, 152244, 152245, 152246, 152247, 152248, 152249, 152250 were selected from the ORFs having the nucleotide sequence represented by SEQ ID NO: 81545.

The probes having the nucleotide sequence represented by SEQ ID NOS: 152251, 152252, 152253, 152254, 152255, 152256, 152257, 152258, 152259, 152260, 152261 were selected from the ORFs having the nucleotide sequence represented by SEQ ID NO: 81546.

The probes having the nucleotide sequence represented by SEQ ID NOS: 152262, 152263, 152264, 152265, 152266, 152267, 152268, 152269, 152270, 152271, 152272 were selected from the ORFs having the nucleotide sequence represented by SEQ ID NO: 81547.

The probes having the nucleotide sequence represented by SEQ ID NOS: 152273, 152274, 152275, 152276, 152277, 152278, 152279, 152280, 152281, 152282, 152283 were selected from the ORFs having the nucleotide sequence represented by SEQ ID NO: 81548.

The probes having the nucleotide sequence represented by SEQ ID NOS: 152284, 152285, 152286, 152287, 152288, 152289, 152290, 152291, 152292, 152293, 152294 were selected from the ORFs having the nucleotide sequence represented by SEQ ID NO: 81549.

The probes having the nucleotide sequence represented by SEQ ID NOS: 152295, 152296, 152297, 152298, 152299, 152300, 152301, 152302, 152303, 152304, 152305 were selected from the ORFs having the nucleotide sequence represented by SEQ ID NO: 81550.

The probes having the nucleotide sequence represented by SEQ ID NOS: 152306, 152307, 152308, 152309, 152310, 152311, 152312, 152313, 152314, 152315, 152316 were selected from the ORFs having the nucleotide sequence represented by SEQ ID NO: 81551.

The probes having the nucleotide sequence represented by SEQ ID NOS: 152317, 152318, 152319, 152320, 152321, 152322, 152323, 152324, 152325, 152326, 152327 were selected from the ORFs having the nucleotide sequence represented by SEQ ID NO: 81552.

The probes having the nucleotide sequence represented by SEQ ID NOS: 152328, 152329, 152330, 152331, 152332, 152333, 152334, 152335, 152336, 152337, 152338 were selected from the ORFs having the nucleotide sequence represented by SEQ ID NO: 81553.

The probes having the nucleotide sequence represented by SEQ ID NOS: 152339, 152340, 152341, 152342, 152343, 152344, 152345, 152346, 152347, 152348, 152349 were selected from the ORFs having the nucleotide sequence represented by SEQ ID NO: 81554.

The probes having the nucleotide sequence represented by SEQ ID NOS: 152350, 152351, 152352, 152353, 152354, 152355, 152356, 152357, 152358, 152359, 152360 were selected from the ORFs having the nucleotide sequence represented by SEQ ID NO: 81555.

The probes having the nucleotide sequence represented by SEQ ID NOS: 152361, 152362, 152363, 152364, 152365, 152366, 152367, 152368, 152369, 152370, 152371 were selected from the ORFs having the nucleotide sequence represented by SEQ ID NO: 81556.

The probes having the nucleotide sequence represented by SEQ ID NOS: 152372, 152373, 152374, 152375, 152376, 152377, 152378, 152379, 152380, 152381, 152382 were selected from the ORFs having the nucleotide sequence represented by SEQ ID NO: 81557.

The probes having the nucleotide sequence represented by SEQ ID NOS: 152383, 152384, 152385, 152386, 152387, 152388, 152389, 152390, 152391, 152392, 152393 were selected from the ORFs having the nucleotide sequence represented by SEQ ID NO: 81558.

The probes having the nucleotide sequence represented by SEQ ID NOS: 152394, 152395, 152396, 152397, 152398, 152399, 152400, 152401, 152402, 152403, 152404 were selected from the ORFs having the nucleotide sequence represented by SEQ ID NO: 81559.

The probes having the nucleotide sequence represented by SEQ ID NOS: 152405, 152406, 152407, 152408, 152409, 152410, 152411, 152412, 152413, 152414, 152415 were selected from the ORFs having the nucleotide sequence represented by SEQ ID NO: 81560.

The probes having the nucleotide sequence represented by SEQ ID NOS: 152416, 152417, 152418, 152419, 152420, 152421, 152422, 152423, 152424, 152425, 152426 were selected from the ORFs having the nucleotide sequence represented by SEQ ID NO: 81561.

The probes having the nucleotide sequence represented by SEQ ID NOS: 152427, 152428, 152429, 152430, 152431, 152432, 152433, 152434, 152435, 152436, 152437 were selected from the ORFs having the nucleotide sequence represented by SEQ ID NO: 81562.

The probes having the nucleotide sequence represented by SEQ ID NOS: 152438, 152439, 152440, 152441, 152442, 152443, 152444, 152445, 152446, 152447, 152448 were selected from the ORFs having the nucleotide sequence represented by SEQ ID NO: 81563.

The probes having the nucleotide sequence represented by SEQ ID NOS: 152449, 152450, 152451, 152452, 152453, 152454, 152455, 152456, 152457, 152458, 152459 were selected from the ORFs having the nucleotide sequence represented by SEQ ID NO: 81564.

The probes having the nucleotide sequence represented by SEQ ID NOS: 152460, 152461, 152462, 152463, 152464, 152465, 152466, 152467, 152468, 152469, 152470, 152471, 152472, 152473, 152474, 152475, 152476, 152477, 152478, 152479, 152480, 152481 were selected from the ORFs having the nucleotide sequence represented by SEQ ID NO: 81565.

The probes having the nucleotide sequence represented by SEQ ID NOS: 152482, 152483, 152484, 152485, 152486, 152487, 152488, 152489, 152490, 152491, 152492 were selected from the ORFs having the nucleotide sequence represented by SEQ ID NO: 81566.

The probes having the nucleotide sequence represented by SEQ ID NOS: 152493, 152494, 152495, 152496, 152497, 152498, 152499, 152500, 152501, 152502, 152503 were selected from the ORFs having the nucleotide sequence represented by SEQ ID NO: 81567.

The probes having the nucleotide sequence represented by SEQ ID NOS: 152504, 152505, 152506, 152507, 152508, 152509, 152510, 152511, 152512, 152513, 152514 were selected from the ORFs having the nucleotide sequence represented by SEQ ID NO: 81568.

The probes having the nucleotide sequence represented by SEQ ID NOS: 152515, 152516, 152517, 152518, 152519, 152520, 152521, 152522, 152523, 152524, 152525 were selected from the ORFs having the nucleotide sequence represented by SEQ ID NO: 81569.

The probes having the nucleotide sequence represented by SEQ ID NOS: 152526, 152527, 152528, 152529, 152530, 152531, 152532, 152533, 152534, 152535, 152536 were selected from the ORFs having the nucleotide sequence represented by SEQ ID NO: 81570.

The probes having the nucleotide sequence represented by SEQ ID NOS: 152537, 152538, 152539, 152540, 152541, 152542, 152543, 152544, 152545, 152546, 152547 were selected from the ORFs having the nucleotide sequence represented by SEQ ID NO: 81571.

The probes having the nucleotide sequence represented by SEQ ID NOS: 152548, 152549, 152550, 152551, 152552, 152553, 152554, 152555, 152556, 152557, 152558 were selected from the ORFs having the nucleotide sequence represented by SEQ ID NO: 81572.

The probes having the nucleotide sequence represented by SEQ ID NOS: 152559, 152560, 152561, 152562, 152563, 152564, 152565, 152566, 152567, 152568, 152569 were selected from the ORFs having the nucleotide sequence represented by SEQ ID NO: 81573.

The probes having the nucleotide sequence represented by SEQ ID NOS: 152570, 152571, 152572, 152573, 152574, 152575, 152576, 152577, 152578, 152579, 152580 were selected from the ORFs having the nucleotide sequence represented by SEQ ID NO: 81574.

The probes having the nucleotide sequence represented by SEQ ID NOS: 152581, 152582, 152583, 152584, 152585, 152586, 152587, 152588, 152589, 152590, 152591were selected from the ORFs having the nucleotide sequence represented by SEQ ID NO: 81575.

The probes having the nucleotide sequence represented by SEQ ID NOS: 152592, 152593, 152594, 152595, 152596, 152597, 152598, 152599, 152600, 152601, 152602 were selected from the ORFs having the nucleotide sequence represented by SEQ ID NO: 81576.

The probes having the nucleotide sequence represented by SEQ ID NOS: 152603, 152604, 152605, 152606, 152607, 152608, 152609, 152610, 152611, 152612, 152613 were selected from the ORFs having the nucleotide sequence represented by SEQ ID NO: 81577.

The probes having the nucleotide sequence represented by SEQ ID NOS: 152614, 152615, 152616, 152617, 152618, 152619, 152620, 152621, 152622, 152623, 152624 were selected from the ORFs having the nucleotide sequence represented by SEQ ID NO: 81578.

The probes having the nucleotide sequence represented by SEQ ID NOS: 152625, 152626, 152627, 152628, 152629, 152630, 152631, 152632, 152633, 152634, 152635 were selected from the ORFs having the nucleotide sequence represented by SEQ ID NO: 81579.

The probes having the nucleotide sequence represented by SEQ ID NOS: 152636, 152637, 152638, 152639, 152640, 152641, 152642, 152643, 152644, 152645, 152646 were selected from the ORFs having the nucleotide sequence represented by SEQ ID NO: 81580.

The probes having the nucleotide sequence represented by SEQ ID NOS: 152647, 152648, 152649, 152650, 152651, 152652, 152653, 152654, 152655, 152656 were selected from the ORFs having the nucleotide sequence represented by SEQ ID NO: 81581.

The probes having the nucleotide sequence represented by SEQ ID NOS: 152657, 152658, 152659, 152660, 152661, 152662, 152663, 152664, 152665, 152666, 152667 were selected from the ORFs having the nucleotide sequence represented by SEQ ID NO: 81582.

The probes having the nucleotide sequence represented by SEQ ID NOS: 152668, 152669, 152670, 152671, 152672, 152673, 152674, 152675, 152676, 152677, 152678 were selected from the ORFs having the nucleotide sequence represented by SEQ ID NO: 81583.

The probes having the nucleotide sequence represented by SEQ ID NOS: 152679, 152680, 152681, 152682, 152683, 152684, 152685, 152686, 152687, 152688, 152689 were selected from the ORFs having the nucleotide sequence represented by SEQ ID NO: 81584.

The probes having the nucleotide sequence represented by SEQ ID NOS: 152690, 152691, 152692, 152693, 152694, 152695, 152696, 152697, 152698, 152699, 152700 were selected from the ORFs having the nucleotide sequence represented by SEQ ID NO: 81585.

The probes having the nucleotide sequence represented by SEQ ID NOS: 152701, 152702, 152703, 152704, 152705, 152706, 152707, 152708, 152709, 152710, 152711 were selected from the ORFs having the nucleotide sequence represented by SEQ ID NO: 81586.

The probes having the nucleotide sequence represented by SEQ ID NOS: 152712, 152713, 152714, 152715, 152716, 152717, 152718, 152719, 152720, 152721, 152722 were selected from the ORFs having the nucleotide sequence represented by SEQ ID NO: 81587.

The probes having the nucleotide sequence represented by SEQ ID NOS: 152723, 152724, 152725, 152726, 152727, 152728, 152729, 152730, 152731, 152732, 152733 were selected from the ORFs having the nucleotide sequence represented by SEQ ID NO: 81588.

The probes having the nucleotide sequence represented by SEQ ID NOS: 152734, 152735, 152736, 152737, 152738, 152739, 152740, 152741, 152742, 152743, 152744 were selected from the ORFs having the nucleotide sequence represented by SEQ ID NO: 81589.

The probes having the nucleotide sequence represented by SEQ ID NOS: 152745, 152746, 152747, 152748, 152749, 152750, 152751, 152752, 152753, 152754, 152755 were selected from the ORFs having the nucleotide sequence represented by SEQ ID NO: 81590.

The probes having the nucleotide sequence represented by SEQ ID NOS: 152756, 152757, 152758, 152759, 152760, 152761, 152762, 152763, 152764, 152765, 152766 were selected from the ORFs having the nucleotide sequence represented by SEQ ID NO: 81591.

The probes having the nucleotide sequence represented by SEQ ID NOS: 152767, 152768, 152769, 152770, 152771, 152772, 152773, 152774, 152775, 152776, 152777 were selected from the ORFs having the nucleotide sequence represented by SEQ ID NO: 81592.

The probes having the nucleotide sequence represented by SEQ ID NOS: 152778, 152779, 152780, 152781, 152782, 152783, 152784, 152785, 152786, 152787, 152788 were selected from the ORFs having the nucleotide sequence represented by SEQ ID NO: 81593.

The probes having the nucleotide sequence represented by SEQ ID NOS: 152789, 152790, 152791, 152792, 152793, 152794, 152795, 152796, 152797, 152798, 152799 were selected from the ORFs having the nucleotide sequence represented by SEQ ID NO: 81594.

The probes having the nucleotide sequence represented by SEQ ID NOS: 152800, 152801, 152802, 152803, 152804, 152805, 152806, 152807, 152808, 152809, 152810 were selected from the ORFs having the nucleotide sequence represented by SEQ ID NO: 81595.

The probes having the nucleotide sequence represented by SEQ ID NOS: 152811, 152812, 152813, 152814, 152815, 152816, 152817, 152818, 152819, 152820, 152821 were selected from the ORFs having the nucleotide sequence represented by SEQ ID NO: 81596.

The probes having the nucleotide sequence represented by SEQ ID NOS: 152822, 152823, 152824, 152825, 152826, 152827, 152828, 152829, 152830, 152831, 152832 were selected from the ORFs having the nucleotide sequence represented by SEQ ID NO: 81597.

The probes having the nucleotide sequence represented by SEQ ID NOS: 152833, 152834, 152835, 152836, 152837, 152838, 152839, 152840, 152841, 152842, 152843 were selected from the ORFs having the nucleotide sequence represented by SEQ ID NO: 81598.

The probes having the nucleotide sequence represented by SEQ ID NOS: 152844, 152845, 152846, 152847, 152848, 152849, 152850, 152851, 152852, 152853, 152854 were selected from the ORFs having the nucleotide sequence represented by SEQ ID NO: 81599.

The probes having the nucleotide sequence represented by SEQ ID NOS: 152855, 152856, 152857, 152858, 152859, 152860, 152861, 152862, 152863, 152864, 152865 were selected from the ORFs having the nucleotide sequence represented by SEQ ID NO: 81600.

The probes having the nucleotide sequence represented by SEQ ID NOS: 152866, 152867, 152868, 152869, 152870, 152871, 152872, 152873, 152874, 152875, 152876 were selected from the ORFs having the nucleotide sequence represented by SEQ ID NO: 81601.

The probes having the nucleotide sequence represented by SEQ ID NOS: 152877, 152878, 152879, 152880, 152881, 152882, 152883, 152884, 152885, 152886, 152887 were selected from the ORFs having the nucleotide sequence represented by SEQ ID NO: 81602.

The probes having the nucleotide sequence represented by SEQ ID NOS: 152888, 152889, 152890, 152891, 152892, 152893, 152894, 152895, 152896, 152897, 152898 were selected from the ORFs having the nucleotide sequence represented by SEQ ID NO: 81603.

The probes having the nucleotide sequence represented by SEQ ID NOS: 152899, 152900, 152901, 152902, 152903, 152904, 152905, 152906, 152907, 152908, 152909 were selected from the ORFs having the nucleotide sequence represented by SEQ ID NO: 81604.

The probes having the nucleotide sequence represented by SEQ ID NOS: 152910, 152911, 152912, 152913, 152914, 152915, 152916, 152917, 152918, 152919, 152920 were selected from the ORFs having the nucleotide sequence represented by SEQ ID NO: 81605.

The probes having the nucleotide sequence represented by SEQ ID NOS: 152921, 152922, 152923, 152924, 152925, 152926, 152927, 152928, 152929, 152930, 152931 were selected from the ORFs having the nucleotide sequence represented by SEQ ID NO: 81606.

The probes having the nucleotide sequence represented by SEQ ID NOS: 152932, 152933, 152934, 152935, 152936, 152937, 152938, 152939, 152940, 152941, 152942 were selected from the ORFs having the nucleotide sequence represented by SEQ ID NO: 81607.

The probes having the nucleotide sequence represented by SEQ ID NOS: 152943, 152944, 152945, 152946, 152947, 152948, 152949, 152950, 152951, 152952, 152953 were selected from the ORFs having the nucleotide sequence represented by SEQ ID NO: 81608.

The probes having the nucleotide sequence represented by SEQ ID NOS: 152954, 152955, 152956, 152957, 152958, 152959, 152960, 152961, 152962, 152963, 152964 were selected from the ORFs having the nucleotide sequence represented by SEQ ID NO: 81609.

The probes having the nucleotide sequence represented by SEQ ID NOS: 152965, 152966, 152967, 152968, 152969, 152970, 152971, 152972, 152973, 152974, 152975 were selected from the ORFs having the nucleotide sequence represented by SEQ ID NO: 81610.

The probes having the nucleotide sequence represented by SEQ ID NOS: 152976, 152977, 152978, 152979, 152980, 152981, 152982, 152983, 152984, 152985, 152986 were selected from the ORFs having the nucleotide sequence represented by SEQ ID NO: 81611.

The probes having the nucleotide sequence represented by SEQ ID NOS: 152987, 152988, 152989, 152990, 152991, 152992, 152993, 152994, 152995, 152996, 152997 were selected from the ORFs having the nucleotide sequence represented by SEQ ID NO: 81612.

The probes having the nucleotide sequence represented by SEQ ID NOS: 152998, 152999, 153000, 153001, 153002, 153003, 153004, 153005, 153006, 153007, 153008 were selected from the ORFs having the nucleotide sequence represented by SEQ ID NO: 81613.

The probes having the nucleotide sequence represented by SEQ ID NOS: 153009, 153010, 153011, 153012, 153013, 153014, 153015, 153016, 153017, 153018, 153019 were selected from the ORFs having the nucleotide sequence represented by SEQ ID NO: 81614.

The probes having the nucleotide sequence represented by SEQ ID NOS: 153020, 153021, 153022, 153023, 153024, 153025, 153026, 153027, 153028, 153029, 153030 were selected from the ORFs having the nucleotide sequence represented by SEQ ID NO: 81615.

The probes having the nucleotide sequence represented by SEQ ID NOS: 153031, 153032, 153033, 153034, 153035, 153036, 153037, 153038, 153039, 153040, 153041 were selected from the ORFs having the nucleotide sequence represented by SEQ ID NO: 81616.

The probes having the nucleotide sequence represented by SEQ ID NOS: 153042, 153043, 153044, 153045, 153046, 153047, 153048, 153049, 153050, 153051, 153052 were selected from the ORFs having the nucleotide sequence represented by SEQ ID NO: 81617.

The probes having the nucleotide sequence represented by SEQ ID NOS: 153053, 153054, 153055, 153056, 153057, 153058, 153059, 153060, 153061, 153062, 153063 were selected from the ORFs having the nucleotide sequence represented by SEQ ID NO: 81618.

The probes having the nucleotide sequence represented by SEQ ID NOS: 153064, 153065, 153066, 153067, 153068, 153069, 153070, 153071, 153072, 153073, 153074, 153075, 153076, 153077, 153078, 153079, 153080, 153081, 153082, 153083, 153084, 153085 were selected from the ORFs having the nucleotide sequence represented by SEQ ID NO: 81619.

The probes having the nucleotide sequence represented by SEQ ID NOS: 153086, 153087, 153088, 153089, 153090, 153091, 153092, 153093, 153094, 153095, 153096 were selected from the ORFs having the nucleotide sequence represented by SEQ ID NO: 81620.

The probes having the nucleotide sequence represented by SEQ ID NOS: 153097, 153098, 153099, 153100, 153101, 153102, 153103, 153104, 153105, 153106, 153107 were selected from the ORFs having the nucleotide sequence represented by SEQ ID NO: 81621.

The probes having the nucleotide sequence represented by SEQ ID NOS: 153108, 153109, 153110, 153111, 153112, 153113, 153114, 153115, 153116, 153117, 153118 were selected from the ORFs having the nucleotide sequence represented by SEQ ID NO: 81622.

The probes having the nucleotide sequence represented by SEQ ID NOS: 153119, 153120, 153121, 153122, 153123, 153124, 153125, 153126, 153127, 153128, 153129 were selected from the ORFs having the nucleotide sequence represented by SEQ ID NO: 81623.

The probes having the nucleotide sequence represented by SEQ ID NOS: 153130, 153131, 153132, 153133, 153134, 153135, 153136, 153137, 153138, 153139, 153140 were selected from the ORFs having the nucleotide sequence represented by SEQ ID NO: 81624.

The probes having the nucleotide sequence represented by SEQ ID NOS: 153141, 153142, 153143, 153144, 153145, 153146, 153147, 153148, 153149, 153150, 153151 were selected from the ORFs having the nucleotide sequence represented by SEQ ID NO: 81625.

The probes having the nucleotide sequence represented by SEQ ID NOS: 153152, 153153, 153154, 153155, 153156, 153157, 153158, 153159, 153160, 153161, 153162 were selected from the ORFs having the nucleotide sequence represented by SEQ ID NO: 81626.

The probes having the nucleotide sequence represented by SEQ ID NOS: 153163, 153164, 153165, 153166, 153167, 153168, 153169, 153170, 153171, 153172, 153173 were selected from the ORFs having the nucleotide sequence represented by SEQ ID NO: 81627.

The probes having the nucleotide sequence represented by SEQ ID NOS: 153174, 153175, 153176, 153177, 153178, 153179, 153180, 153181, 153182, 153183, 153184 were selected from the ORFs having the nucleotide sequence represented by SEQ ID NO: 81628.

The probes having the nucleotide sequence represented by SEQ ID NOS: 153185, 153186, 153187, 153188, 153189, 153190, 153191, 153192, 153193, 153194, 153195 were selected from the ORFs having the nucleotide sequence represented by SEQ ID NO: 81629.

The probes having the nucleotide sequence represented by SEQ ID NOS: 153196, 153197, 153198, 153199, 153200, 153201, 153202, 153203, 153204, 153205, 153206 were selected from the ORFs having the nucleotide sequence represented by SEQ ID NO: 81630.

The probes having the nucleotide sequence represented by SEQ ID NOS: 153207, 153208, 153209, 153210, 153211, 153212, 153213, 153214, 153215, 153216, 153217 were selected from the ORFs having the nucleotide sequence represented by SEQ ID NO: 81631.

The probes having the nucleotide sequence represented by SEQ ID NOS: 153218, 153219, 153220, 153221, 153222, 153223, 153224, 153225, 153226, 153227, 153228 were selected from the ORFs having the nucleotide sequence represented by SEQ ID NO: 81632.

The probes having the nucleotide sequence represented by SEQ ID NOS: 153229, 153230, 153231, 153232, 153233, 153234, 153235, 153236, 153237, 153238, 153239 were selected from the ORFs having the nucleotide sequence represented by SEQ ID NO: 81633.

The probes having the nucleotide sequence represented by SEQ ID NOS: 153240, 153241, 153242, 153243, 153244, 153245, 153246, 153247, 153248, 153249, 153250 were selected from the ORFs having the nucleotide sequence represented by SEQ ID NO: 81634.

The probes having the nucleotide sequence represented by SEQ ID NOS: 153251, 153252, 153253, 153254, 153255, 153256, 153257, 153258, 153259, 153260, 153261 were selected from the ORFs having the nucleotide sequence represented by SEQ ID NO: 81635.

The probes having the nucleotide sequence represented by SEQ ID NOS: 153262, 153263, 153264, 153265, 153266, 153267, 153268, 153269, 153270, 153271, 153272 were selected from the ORFs having the nucleotide sequence represented by SEQ ID NO: 81636.

The probes having the nucleotide sequence represented by SEQ ID NOS: 153273, 153274, 153275, 153276, 153277, 153278, 153279, 153280, 153281, 153282, 153283 were selected from the ORFs having the nucleotide sequence represented by SEQ ID NO: 81637.

The probes having the nucleotide sequence represented by SEQ ID NOS: 153284, 153285, 153286, 153287, 153288, 153289, 153290, 153291, 153292, 153293, 153294 were selected from the ORFs having the nucleotide sequence represented by SEQ ID NO: 81638.

The probes having the nucleotide sequence represented by SEQ ID NOS: 153295, 153296, 153297, 153298, 153299, 153300, 153301, 153302, 153303, 153304, 153305 were selected from the ORFs having the nucleotide sequence represented by SEQ ID NO: 81639.

The probes having the nucleotide sequence represented by SEQ ID NOS: 153306, 153307, 153308, 153309, 153310, 153311, 153312, 153313, 153314, 153315, 153316 were selected from the ORFs having the nucleotide sequence represented by SEQ ID NO: 81640.

The probes having the nucleotide sequence represented by SEQ ID NOS: 153317, 153318, 153319, 153320, 153321, 153322, 153323, 153324, 153325, 153326, 153327 were selected from the ORFs having the nucleotide sequence represented by SEQ ID NO: 81641.

The probes having the nucleotide sequence represented by SEQ ID NOS: 153328, 153329, 153330, 153331, 153332, 153333, 153334, 153335, 153336, 153337, 153338 were selected from the ORFs having the nucleotide sequence represented by SEQ ID NO: 81642.

The probes having the nucleotide sequence represented by SEQ ID NOS: 153339, 153340, 153341, 153342, 153343, 153344, 153345, 153346, 153347, 153348, 153349 were selected from the ORFs having the nucleotide sequence represented by SEQ ID NO: 81643.

The probes having the nucleotide sequence represented by SEQ ID NOS: 153350, 153351, 153352, 153353, 153354, 153355, 153356, 153357, 153358, 153359, 153360 were selected from the ORFs having the nucleotide sequence represented by SEQ ID NO: 81644.

The probes having the nucleotide sequence represented by SEQ ID NOS: 153361, 153362, 153363, 153364, 153365, 153366, 153367, 153368, 153369, 153370, 153371 were selected from the ORFs having the nucleotide sequence represented by SEQ ID NO: 81645.

The probes having the nucleotide sequence represented by SEQ ID NOS: 153372, 153373, 153374, 153375, 153376, 153377, 153378, 153379, 153380, 153381, 153382 were selected from the ORFs having the nucleotide sequence represented by SEQ ID NO: 81646.

The probes having the nucleotide sequence represented by SEQ ID NOS: 153383, 153384, 153385, 153386, 153387, 153388, 153389, 153390, 153391, 153392, 153393 were selected from the ORFs having the nucleotide sequence represented by SEQ ID NO: 81647.

The probes having the nucleotide sequence represented by SEQ ID NOS: 153394, 153395, 153396, 153397, 153398, 153399, 153400, 153401, 153402, 153403, 153404 were selected from the ORFs having the nucleotide sequence represented by SEQ ID NO: 81648.

The probes having the nucleotide sequence represented by SEQ ID NOS: 153405, 153406, 153407, 153408, 153409, 153410, 153411, 153412, 153413, 153414, 153415 were selected from the ORFs having the nucleotide sequence represented by SEQ ID NO: 81649.

The probes having the nucleotide sequence represented by SEQ ID NOS: 153416, 153417, 153418, 153419, 153420, 153421, 153422, 153423, 153424, 153425, 153426 were selected from the ORFs having the nucleotide sequence represented by SEQ ID NO: 81650.

The probes having the nucleotide sequence represented by SEQ ID NOS: 153427, 153428, 153429, 153430, 153431, 153432, 153433, 153434, 153435, 153436, 153437 were selected from the ORFs having the nucleotide sequence represented by SEQ ID NO: 81651.

The probes having the nucleotide sequence represented by SEQ ID NOS: 153438, 153439, 153440, 153441, 153442, 153443, 153444, 153445, 153446, 153447, 153448 were selected from the ORFs having the nucleotide sequence represented by SEQ ID NO: 81652.

The probes having the nucleotide sequence represented by SEQ ID NOS: 153449, 153450, 153451, 153452, 153453, 153454, 153455, 153456, 153457, 153458, 153459 were selected from the ORFs having the nucleotide sequence represented by SEQ ID NO: 81653.

The probes having the nucleotide sequence represented by SEQ ID NOS: 153460, 153461, 153462, 153463, 153464, 153465, 153466, 153467, 153468, 153469, 153470 were selected from the ORFs having the nucleotide sequence represented by SEQ ID NO: 81654.

The probes having the nucleotide sequence represented by SEQ ID NOS: 153471, 153472, 153473, 153474, 153475, 153476, 153477, 153478, 153479, 153480, 153481 were selected from the ORFs having the nucleotide sequence represented by SEQ ID NO: 81655.

The probes having the nucleotide sequence represented by SEQ ID NOS: 153482, 153483, 153484, 153485, 153486, 153487, 153488, 153489, 153490, 153491, 153492 were selected from the ORFs having the nucleotide sequence represented by SEQ ID NO: 81656.

The probes having the nucleotide sequence represented by SEQ ID NOS: 153493, 153494, 153495, 153496, 153497, 153498, 153499, 153500, 153501, 153502, 153503 were selected from the ORFs having the nucleotide sequence represented by SEQ ID NO: 81657.

The probes having the nucleotide sequence represented by SEQ ID NOS: 153504, 153505, 153506, 153507, 153508, 153509, 153510, 153511, 153512, 153513, 153514 were selected from the ORFs having the nucleotide sequence represented by SEQ ID NO: 81658.

The probes having the nucleotide sequence represented by SEQ ID NOS: 153515, 153516, 153517, 153518, 153519, 153520, 153521, 153522, 153523, 153524, 153525 were selected from the ORFs having the nucleotide sequence represented by SEQ ID NO: 81659.

The probes having the nucleotide sequence represented by SEQ ID NOS: 153526, 153527, 153528, 153529, 153530, 153531, 153532, 153533, 153534, 153535, 153536 were selected from the ORFs having the nucleotide sequence represented by SEQ ID NO: 81660.

The probes having the nucleotide sequence represented by SEQ ID NOS: 153537, 153538, 153539, 153540, 153541, 153542, 153543, 153544, 153545, 153546, 153547 were selected from the ORFs having the nucleotide sequence represented by SEQ ID NO: 81661.

The probes having the nucleotide sequence represented by SEQ ID NOS: 153548, 153549, 153550, 153551, 153552, 153553, 153554, 153555, 153556, 153557, 153558 were selected from the ORFs having the nucleotide sequence represented by SEQ ID NO: 81662.

The probes having the nucleotide sequence represented by SEQ ID NOS: 153559, 153560, 153561, 153562, 153563, 153564, 153565, 153566, 153567, 153568, 153569 were selected from the ORFs having the nucleotide sequence represented by SEQ ID NO: 81663.

The probes having the nucleotide sequence represented by SEQ ID NOS: 153570, 153571, 153572, 153573, 153574, 153575, 153576, 153577, 153578, 153579, 153580 were selected from the ORFs having the nucleotide sequence represented by SEQ ID NO: 81664.

The probes having the nucleotide sequence represented by SEQ ID NOS: 153581, 153582, 153583, 153584, 153585, 153586, 153587, 153588, 153589, 153590, 153591 were selected from the ORFs having the nucleotide sequence represented by SEQ ID NO: 81665.

The probes having the nucleotide sequence represented by SEQ ID NOS: 153592, 153593, 153594, 153595, 153596, 153597, 153598, 153599, 153600, 153601, 153602 were selected from the ORFs having the nucleotide sequence represented by SEQ ID NO: 81666.

The probes having the nucleotide sequence represented by SEQ ID NOS: 153603, 153604, 153605, 153606, 153607, 153608, 153609, 153610, 153611, 153612, 153613 were selected from the ORFs having the nucleotide sequence represented by SEQ ID NO: 81667.

The probes having the nucleotide sequence represented by SEQ ID NOS: 153614, 153615, 153616, 153617, 153618, 153619, 153620, 153621, 153622, 153623, 153624 were selected from the ORFs having the nucleotide sequence represented by SEQ ID NO: 81668.

The probes having the nucleotide sequence represented by SEQ ID NOS: 153625, 153626, 153627, 153628, 153629, 153630, 153631, 153632, 153633, 153634, 153635 were selected from the ORFs having the nucleotide sequence represented by SEQ ID NO: 81669.

The probes having the nucleotide sequence represented by SEQ ID NOS: 153636, 153637, 153638, 153639, 153640, 153641, 153642, 153643, 153644, 153645, 153646 were selected from the ORFs having the nucleotide sequence represented by SEQ ID NO: 81670.

The probes having the nucleotide sequence represented by SEQ ID NOS: 153647, 153648, 153649, 153650, 153651, 153652, 153653, 153654, 153655, 153656, 153657 were selected from the ORFs having the nucleotide sequence represented by SEQ ID NO: 81671.

The probes having the nucleotide sequence represented by SEQ ID NOS: 153658, 153659, 153660, 153661, 153662, 153663, 153664, 153665, 153666, 153667, 153668 were selected from the ORFs having the nucleotide sequence represented by SEQ ID NO: 81672.

The probes having the nucleotide sequence represented by SEQ ID NOS: 153669, 153670, 153671, 153672, 153673, 153674, 153675, 153676, 153677, 153678, 153679 were selected from the ORFs having the nucleotide sequence represented by SEQ ID NO: 81673.

The probes having the nucleotide sequence represented by SEQ ID NOS: 153680, 153681, 153682, 153683, 153684, 153685, 153686, 153687, 153688, 153689, 153690 were selected from the ORFs having the nucleotide sequence represented by SEQ ID NO: 81674.

The probes having the nucleotide sequence represented by SEQ ID NOS: 153691, 153692, 153693, 153694, 153695, 153696, 153697, 153698, 153699, 153700, 153701 were selected from the ORFs having the nucleotide sequence represented by SEQ ID NO: 81675.

The probes having the nucleotide sequence represented by SEQ ID NOS: 153702, 153703, 153704, 153705, 153706, 153707, 153708, 153709, 153710, 153711, 153712 were selected from the ORFs having the nucleotide sequence represented by SEQ ID NO: 81676.

The probes having the nucleotide sequence represented by SEQ ID NOS: 153713, 153714, 153715, 153716, 153717, 153718, 153719, 153720, 153721, 153722, 153723 were selected from the ORFs having the nucleotide sequence represented by SEQ ID NO: 81677.

The probes having the nucleotide sequence represented by SEQ ID NOS: 153724, 153725, 153726, 153727, 153728, 153729, 153730, 153731, 153732, 153733, 153734 were selected from the ORFs having the nucleotide sequence represented by SEQ ID NO: 81678.

The probes having the nucleotide sequence represented by SEQ ID NOS: 153735, 153736, 153737, 153738, 153739, 153740, 153741, 153742, 153743, 153744, 153745 were selected from the ORFs having the nucleotide sequence represented by SEQ ID NO: 81679.

The probes having the nucleotide sequence represented by SEQ ID NOS: 153746, 153747, 153748, 153749, 153750, 153751, 153752, 153753, 153754, 153755, 153756 were selected from the ORFs having the nucleotide sequence represented by SEQ ID NO: 81680.

The probes having the nucleotide sequence represented by SEQ ID NOS: 153757, 153758, 153759, 153760, 153761, 153762, 153763, 153764, 153765, 153766, 153767 were selected from the ORFs having the nucleotide sequence represented by SEQ ID NO: 81681.

The probes having the nucleotide sequence represented by SEQ ID NOS: 153768, 153769, 153770, 153771, 153772, 153773, 153774, 153775, 153776, 153777, 153778 were selected from the ORFs having the nucleotide sequence represented by SEQ ID NO: 81682.

The probes having the nucleotide sequence represented by SEQ ID NOS: 153779, 153780, 153781, 153782, 153783, 153784, 153785, 153786, 153787, 153788, 153789 were selected from the ORFs having the nucleotide sequence represented by SEQ ID NO: 81683.

The probes having the nucleotide sequence represented by SEQ ID NOS: 153790, 153791, 153792, 153793, 153794, 153795, 153796, 153797, 153798, 153799, 153800 were selected from the ORFs having the nucleotide sequence represented by SEQ ID NO: 81684.

The probes having the nucleotide sequence represented by SEQ ID NOS: 153801, 153802, 153803, 153804, 153805, 153806, 153807, 153808, 153809, 153810, 153811 were selected from the ORFs having the nucleotide sequence represented by SEQ ID NO: 81685.

The probes having the nucleotide sequence represented by SEQ ID NOS: 153812, 153813, 153814, 153815, 153816, 153817, 153818, 153819, 153820, 153821, 153822 were selected from the ORFs having the nucleotide sequence represented by SEQ ID NO: 81686.

The probes having the nucleotide sequence represented by SEQ ID NOS: 153823, 153824, 153825, 153826, 153827, 153828, 153829, 153830, 153831, 153832, 153833 were selected from the ORFs having the nucleotide sequence represented by SEQ ID NO: 81687.

The probes having the nucleotide sequence represented by SEQ ID NOS: 153834, 153835, 153836, 153837, 153838, 153839, 153840, 153841, 153842, 153843, 153844 were selected from the ORFs having the nucleotide sequence represented by SEQ ID NO: 81688.

The probes having the nucleotide sequence represented by SEQ ID NOS: 153845, 153846, 153847, 153848, 153849, 153850, 153851, 153852, 153853, 153854, 153855 were selected from the ORFs having the nucleotide sequence represented by SEQ ID NO: 81689.

The probes having the nucleotide sequence represented by SEQ ID NOS: 153856, 153857, 153858, 153859, 153860, 153861, 153862, 153863, 153864, 153865, 153866 were selected from the ORFs having the nucleotide sequence represented by SEQ ID NO: 81690.

The probes having the nucleotide sequence represented by SEQ ID NOS: 153867, 153868, 153869, 153870, 153871, 153872, 153873, 153874, 153875, 153876, 153877 were selected from the ORFs having the nucleotide sequence represented by SEQ ID NO: 81691.

The probes having the nucleotide sequence represented by SEQ ID NOS: 153878, 153879, 153880, 153881, 153882, 153883, 153884, 153885, 153886 were selected from the ORFs having the nucleotide sequence represented by SEQ ID NO: 81692.

The probes having the nucleotide sequence represented by SEQ ID NOS: 153887, 153888, 153889, 153890, 153891, 153892, 153893, 153894, 153895, 153896, 153897 were selected from the ORFs having the nucleotide sequence represented by SEQ ID NO: 81693.

The probes having the nucleotide sequence represented by SEQ ID NOS: 153898, 153899, 153900, 153901, 153902, 153903, 153904, 153905, 153906, 153907, 153908 were selected from the ORFs having the nucleotide sequence represented by SEQ ID NO: 81694.

The probes having the nucleotide sequence represented by SEQ ID NOS: 153909, 153910, 153911, 153912, 153913, 153914, 153915, 153916, 153917, 153918, 153919 were selected from the ORFs having the nucleotide sequence represented by SEQ ID NO: 81695.

The probes having the nucleotide sequence represented by SEQ ID NOS: 153920, 153921, 153922, 153923, 153924, 153925, 153926, 153927, 153928, 153929, 153930 were selected from the ORFs having the nucleotide sequence represented by SEQ ID NO: 81696.

The probes having the nucleotide sequence represented by SEQ ID NOS: 153931, 153932, 153933, 153934, 153935, 153936, 153937, 153938, 153939, 153940, 153941 were selected from the ORFs having the nucleotide sequence represented by SEQ ID NO: 81697.

The probes having the nucleotide sequence represented by SEQ ID NOS: 153942, 153943, 153944, 153945, 153946, 153947, 153948, 153949, 153950, 153951, 153952 were selected from the ORFs having the nucleotide sequence represented by SEQ ID NO: 81698.

The probes having the nucleotide sequence represented by SEQ ID NOS: 153953, 153954, 153955, 153956, 153957, 153958, 153959, 153960, 153961, 153962, 153963 were selected from the ORFs having the nucleotide sequence represented by SEQ ID NO: 81699.

The probes having the nucleotide sequence represented by SEQ ID NOS: 153964, 153965, 153966, 153967, 153968, 153969, 153970, 153971, 153972, 153973, 153974 were selected from the ORFs having the nucleotide sequence represented by SEQ ID NO: 81700.

The probes having the nucleotide sequence represented by SEQ ID NOS: 153975, 153976, 153977, 153978, 153979, 153980, 153981, 153982, 153983, 153984, 153985 were selected from the ORFs having the nucleotide sequence represented by SEQ ID NO: 81701.

The probes having the nucleotide sequence represented by SEQ ID NOS: 153986, 153987, 153988, 153989, 153990, 153991, 153992, 153993, 153994, 153995, 153996 were selected from the ORFs having the nucleotide sequence represented by SEQ ID NO: 81702.

The probes having the nucleotide sequence represented by SEQ ID NOS: 153997, 153998, 153999, 154000, 154001, 154002, 154003, 154004, 154005, 154006, 154007 were selected from the ORFs having the nucleotide sequence represented by SEQ ID NO: 81703.

The probes having the nucleotide sequence represented by SEQ ID NOS: 154008, 154009, 154010, 154011, 154012, 154013, 154014, 154015, 154016, 154017, 154018 were selected from the ORFs having the nucleotide sequence represented by SEQ ID NO: 81704.

The probes having the nucleotide sequence represented by SEQ ID NOS: 154019, 154020, 154021, 154022, 154023, 154024, 154025, 154026, 154027, 154028, 154029 were selected from the ORFs having the nucleotide sequence represented by SEQ ID NO: 81705.

The probes having the nucleotide sequence represented by SEQ ID NOS: 154030, 154031, 154032, 154033, 154034, 154035, 154036, 154037, 154038, 154039, 154040 were selected from the ORFs having the nucleotide sequence represented by SEQ ID NO: 81706.

The probes having the nucleotide sequence represented by SEQ ID NOS: 154041, 154042, 154043, 154044, 154045, 154046, 154047, 154048, 154049, 154050, 154051 were selected from the ORFs having the nucleotide sequence represented by SEQ ID NO: 81707.

The probes having the nucleotide sequence represented by SEQ ID NOS: 154052, 154053, 154054, 154055, 154056, 154057, 154058, 154059, 154060, 154061, 154062 were selected from the ORFs having the nucleotide sequence represented by SEQ ID NO: 81708.

The probes having the nucleotide sequence represented by SEQ ID NOS: 154063, 154064, 154065, 154066, 154067, 154068, 154069, 154070, 154071, 154072, 154073 were selected from the ORFs having the nucleotide sequence represented by SEQ ID NO: 81709.

The probes having the nucleotide sequence represented by SEQ ID NOS: 154074, 154075, 154076, 154077, 154078, 154079, 154080, 154081, 154082, 154083, 154084 were selected from the ORFs having the nucleotide sequence represented by SEQ ID NO: 81710.

The probes having the nucleotide sequence represented by SEQ ID NOS: 154085, 154086, 154087, 154088, 154089, 154090, 154091, 154092, 154093, 154094, 154095 were selected from the ORFs having the nucleotide sequence represented by SEQ ID NO: 81711.

The probes having the nucleotide sequence represented by SEQ ID NOS: 154096, 154097, 154098, 154099, 154100, 154101, 154102, 154103, 154104, 154105, 154106 were selected from the ORFs having the nucleotide sequence represented by SEQ ID NO: 81712.

The probes having the nucleotide sequence represented by SEQ ID NOS: 154107, 154108, 154109, 154110, 154111, 154112, 154113, 154114, 154115, 154116, 154117 were selected from the ORFs having the nucleotide sequence represented by SEQ ID NO: 81713.

The probes having the nucleotide sequence represented by SEQ ID NOS: 154118, 154119, 154120, 154121, 154122, 154123, 154124, 154125, 154126, 154127, 154128 were selected from the ORFs having the nucleotide sequence represented by SEQ ID NO: 81714.

The probes having the nucleotide sequence represented by SEQ ID NOS: 154129, 154130, 154131, 154132, 154133, 154134, 154135, 154136, 154137, 154138, 154139 were selected from the ORFs having the nucleotide sequence represented by SEQ ID NO: 81715.

The probes having the nucleotide sequence represented by SEQ ID NOS: 154140, 154141, 154142, 154143, 154144, 154145, 154146, 154147, 154148, 154149, 154150 were selected from the ORFs having the nucleotide sequence represented by SEQ ID NO: 81716.

The probes having the nucleotide sequence represented by SEQ ID NOS: 154151, 154152, 154153, 154154, 154155, 154156, 154157, 1541,58, 154159, 154160, 154161 were selected from the ORFs having the nucleotide sequence represented by SEQ ID NO: 81717.

The probes having the nucleotide sequence represented by SEQ ID NOS: 154162, 154163, 154164, 154165, 154166, 154167, 154168, 154169, 154170, 154171, 154172 were selected from the ORFs having the nucleotide sequence represented by SEQ ID NO: 81718.

The probes having the nucleotide sequence represented by SEQ ID NOS: 154173, 154174, 154175, 154176, 154177, 154178, 154179, 154180, 154181, 154182, 154183 were selected from the ORFs having the nucleotide sequence represented by SEQ ID NO: 81719.

The probes having the nucleotide sequence represented by SEQ ID NOS: 154184, 154185, 154186, 154187, 154188, 154189, 154190, 154191, 154192, 154193, 154194 were selected from the ORFs having the nucleotide sequence represented by SEQ ID NO: 81720.

The probes having the nucleotide sequence represented by SEQ ID NOS: 154195, 154196, 154197, 154198, 154199, 154200, 154201, 154202, 154203, 154204, 154205 were selected from the ORFs having the nucleotide sequence represented by SEQ ID NO: 81721.

The probes having the nucleotide sequence represented by SEQ ID NOS: 154206, 154207, 154208, 154209, 154210, 154211, 154212, 154213, 154214, 154215, 154216 were selected from the ORFs having the nucleotide sequence represented by SEQ ID NO: 81722.

The probes having the nucleotide sequence represented by SEQ ID NOS: 154217, 154218, 154219, 154220, 154221, 154222, 154223, 154224, 154225, 154226, 154227 were selected from the ORFs having the nucleotide sequence represented by SEQ ID NO: 81723.

The probes having the nucleotide sequence represented by SEQ ID NOS: 154228, 154229, 154230, 154231, 154232, 154233, 154234, 154235, 154236, 154237, 154238 were selected from the ORFs having the nucleotide sequence represented by SEQ ID NO: 81724.

The probes having the nucleotide sequence represented by SEQ ID NOS: 154239, 154240, 154241, 154242, 154243, 154244, 154245, 154246, 154247, 154248, 154249 were selected from the ORFs having the nucleotide sequence represented by SEQ ID NO: 81725.

The probes having the nucleotide sequence represented by SEQ ID NOS: 154250, 154251, 154252, 154253, 154254, 154255, 154256, 154257, 154258, 154259, 154260 were selected from the ORFs having the nucleotide sequence represented by SEQ ID NO: 81726.

The probes having the nucleotide sequence represented by SEQ ID NOS: 154261, 154262, 154263, 154264, 154265, 154266, 154267, 154268, 154269, 154270, 154271 were selected from the ORFs having the nucleotide sequence represented by SEQ ID NO: 81727.

The probes having the nucleotide sequence represented by SEQ ID NOS: 154272, 154273, 154274, 154275, 154276, 154277, 154278, 154279, 154280, 154281, 154282 were selected from the ORFs having the nucleotide sequence represented by SEQ ID NO: 81728.

The probes having the nucleotide sequence represented by SEQ ID NOS: 154283, 154284, 154285, 154286, 154287, 154288, 154289, 154290, 154291, 154292, 154293 were selected from the ORFs having the nucleotide sequence represented by SEQ ID NO: 81729.

The probes having the nucleotide sequence represented by SEQ ID NOS: 154294, 154295, 154296, 154297, 154298, 154299, 154300, 154301, 154302, 154303, 154304 were selected from the ORFs having the nucleotide sequence represented by SEQ ID NO: 81730.

The probes having the nucleotide sequence represented by SEQ ID NOS: 154305, 154306, 154307, 154308, 154309, 154310, 154311, 154312, 154313, 154314, 154315 were selected from the ORFs having the nucleotide sequence represented by SEQ ID NO: 81731.

The probes having the nucleotide sequence represented by SEQ ID NOS: 154316, 154317, 154318, 154319, 154320, 154321, 154322, 154323, 154324, 154325, 154326 were selected from the ORFs having the nucleotide sequence represented by SEQ ID NO: 81732.

The probes having the nucleotide sequence represented by SEQ ID NOS: 154327, 154328, 154329, 154330, 154331, 154332, 154333, 154334, 154335, 154336, 154337 were selected from the ORFs having the nucleotide sequence represented by SEQ ID NO: 81733.

The probes having the nucleotide sequence represented by SEQ ID NOS: 154338, 154339, 154340, 154341, 154342, 154343, 154344, 154345, 154346, 154347, 154348 were selected from the ORFs having the nucleotide sequence represented by SEQ ID NO: 81734.

The probes having the nucleotide sequence represented by SEQ ID NOS: 154349, 154350, 154351, 154352, 154353, 154354, 154355, 154356, 154357, 154358, 154359, 154360, 154361, 154362, 154363, 154364, 154365, 154366, 154367, 154368, 154369, 154370 were selected from the ORFs having the nucleotide sequence represented by SEQ ID NO: 81735.

The probes having the nucleotide sequence represented by SEQ ID NOS: 154371, 154372, 154373, 154374, 154375, 154376, 154377, 154378, 154379, 154380, 154381 were selected from the ORFs having the nucleotide sequence represented by SEQ ID NO: 81736.

The probes having the nucleotide sequence represented by SEQ ID NOS: 154382, 154383, 154384, 154385, 154386, 154387, 154388, 154389, 154390, 154391, 154392 were selected from the ORFs having the nucleotide sequence represented by SEQ ID NO: 81737.

The probes having the nucleotide sequence represented by SEQ ID NOS: 154393, 154394, 154395, 154396, 154397, 154398, 154399, 154400, 154401, 154402, 154403, 154404, 154405, 154406, 154407, 154408, 154409, 154410, 154411, 154412, 154413, 154414 were selected from the ORFs having the nucleotide sequence represented by SEQ ID NO: 81738.

The probes having the nucleotide sequence represented by SEQ ID NOS: 154415, 154416, 154417, 154418, 154419, 154420, 154421, 154422, 154423, 154424, 154425, 154426, 154427, 154428, 154429, 154430, 154431, 154432, 154433, 154434, 154435, 154436 were selected from the ORFs having the nucleotide sequence represented by SEQ ID NO: 81739.

The probes having the nucleotide sequence represented by SEQ ID NOS: 154437, 154438, 154439, 154440, 154441, 154442, 154443, 154444, 154445, 154446, 154447 were selected from the ORFs having the nucleotide sequence represented by SEQ ID NO: 81740.

The probes having the nucleotide sequence represented by SEQ ID NOS: 154448, 154449, 154450, 154451, 154452, 154453, 154454, 154455, 154456, 154457, 154458 were selected from the ORFs having the nucleotide sequence represented by SEQ ID NO: 81741.

The probes having the nucleotide sequence represented by SEQ ID NOS: 154459, 154460, 154461, 154462, 154463, 154464, 154465, 154466, 154467, 154468, 154469 were selected from the ORFs having the nucleotide sequence represented by SEQ ID NO: 81742.

The probes having the nucleotide sequence represented by SEQ ID NOS: 154470, 154471, 154472, 154473, 154474, 154475, 154476, 154477, 154478, 154479, 154480 were selected from the ORFs having the nucleotide sequence represented by SEQ ID NO: 81743.

The probes having the nucleotide sequence represented by SEQ ID NOS: 154481, 154482, 154483, 154484, 154485, 154486, 154487, 154488, 154489, 154490, 154491 were selected from the ORFs having the nucleotide sequence represented by SEQ ID NO: 81744.

The probes having the nucleotide sequence represented by SEQ ID NOS: 154492, 154493, 154494, 154495, 154496, 154497, 154498, 154499, 154500, 154501, 154502 were selected from the ORFs having the nucleotide sequence represented by SEQ ID NO: 81745.

The probes having the nucleotide sequence represented by SEQ ID NOS: 154503, 154504, 154505, 154506, 154507, 154508, 154509, 154510, 154511, 154512, 154513 were selected from the ORFs having the nucleotide sequence represented by SEQ ID NO: 81746.

The probes having the nucleotide sequence represented by SEQ ID NOS: 154514, 154515, 154516, 154517, 154518, 154519, 154520, 154521, 154522, 154523, 154524 were selected from the ORFs having the nucleotide sequence represented by SEQ ID NO: 81747.

The probes having the nucleotide sequence represented by SEQ ID NOS: 154525, 154526, 154527, 154528, 154529, 154530, 154531, 154532, 154533, 154534, 154535 were selected from the ORFs having the nucleotide sequence represented by SEQ ID NO: 81748.

The probes having the nucleotide sequence represented by SEQ ID NOS: 154536, 154537, 154538, 154539, 154540, 154541, 154542, 154543, 154544, 154545, 154546 were selected from the ORFs having the nucleotide sequence represented by SEQ ID NO: 81749.

The probes having the nucleotide sequence represented by SEQ ID NOS: 154547, 154548, 154549, 154550, 154551, 154552, 154553, 154554, 154555, 154556, 154557 were selected from the ORFs having the nucleotide sequence represented by SEQ ID NO: 81750.

The probes having the nucleotide sequence represented by SEQ ID NOS: 154558, 154559, 154560, 154561, 154562, 154563, 154564, 154565, 154566, 154567, 154568 were selected from the ORFs having the nucleotide sequence represented by SEQ ID NO: 81751.

The probes having the nucleotide sequence represented by SEQ ID NOS: 154569, 154570, 154571, 154572, 154573, 154574, 154575, 154576, 154577, 154578, 154579 were selected from the ORFs having the nucleotide sequence represented by SEQ ID NO: 81752.

The probes having the nucleotide sequence represented by SEQ ID NOS: 154580, 154581, 154582, 154583, 154584, 154585, 154586, 154587, 154588, 154589, 154590 were selected from the ORFs having the nucleotide sequence represented by SEQ ID NO: 81753.

The probes having the nucleotide sequence represented by SEQ ID NOS: 154591, 154592, 154593, 154594, 154595, 154596, 154597, 154598, 154599, 154600, 154601 were selected from the ORFs having the nucleotide sequence represented by SEQ ID NO: 81754.

The probes having the nucleotide sequence represented by SEQ ID NOS: 154602, 154603, 154604, 154605, 154606, 154607, 154608, 154609, 154610, 154611, 154612 were selected from the ORFs having the nucleotide sequence represented by SEQ ID NO: 81755.

The probes having the nucleotide sequence represented by SEQ ID NOS: 154613, 154614, 154615, 154616, 154617, 154618, 154619, 154620, 154621, 154622, 154623 were selected from the ORFs having the nucleotide sequence represented by SEQ ID NO: 81756.

The probes having the nucleotide sequence represented by SEQ ID NOS: 154624, 154625, 154626, 154627, 154628, 154629, 154630, 154631, 154632, 154633, 154634 were selected from the ORFs having the nucleotide sequence represented by SEQ ID NO: 81757.

The probes having the nucleotide sequence represented by SEQ ID NOS: 154635, 154636, 154637, 154638, 154639, 154640, 154641, 154642, 154643, 154644, 154645 were selected from the ORFs having the nucleotide sequence represented by SEQ ID NO: 81758.

The probes having the nucleotide sequence represented by SEQ ID NOS: 154646, 154647, 154648, 154649, 154650, 154651, 154652, 154653, 154654, 154655, 154656 were selected from the ORFs having the nucleotide sequence represented by SEQ ID NO: 81759.

The probes having the nucleotide sequence represented by SEQ ID NOS: 154657, 154658, 154659, 154660, 154661, 154662, 154663, 154664, 154665, 154666, 154667 were selected from the ORFs having the nucleotide sequence represented by SEQ ID NO: 81760.

The probes having the nucleotide sequence represented by SEQ ID NOS: 154668, 154669, 154670, 154671, 154672, 154673, 154674, 154675, 154676, 154677, 154678 were selected from the ORFs having the nucleotide sequence represented by SEQ ID NO: 81761.

The probes having the nucleotide sequence represented by SEQ ID NOS: 154679, 154680, 154681, 154682, 154683, 154684, 154685, 154686, 154687, 154688, 154689 were selected from the ORFs having the nucleotide sequence represented by SEQ ID NO: 81762.

The probes having the nucleotide sequence represented by SEQ ID NOS: 154690, 154691, 154692, 154693, 154694, 154695, 154696, 154697, 154698, 154699, 154700 were selected from the ORFs having the nucleotide sequence represented by SEQ ID NO: 81763.

The probes having the nucleotide sequence represented by SEQ ID NOS: 154701, 154702, 154703, 154704, 154705, 154706, 154707, 154708, 154709, 154710, 154711 were selected from the ORFs having the nucleotide sequence represented by SEQ ID NO: 81764.

The probes having the nucleotide sequence represented by SEQ ID NOS: 154712, 154713, 154714, 154715, 154716, 154717, 154718, 154719, 154720, 154721, 154722 were selected from the ORFs having the nucleotide sequence represented by SEQ ID NO: 81765.

The probes having the nucleotide sequence represented by SEQ ID NOS: 154723, 154724, 154725, 154726, 154727, 154728, 154729, 154730, 154731, 154732, 154733 were selected from the ORFs having the nucleotide sequence represented by SEQ ID NO: 81766.

The probes having the nucleotide sequence represented by SEQ ID NOS: 154734, 154735, 154736, 154737, 154738, 154739, 154740, 154741, 154742, 154743, 154744 were selected from the ORFs having the nucleotide sequence represented by SEQ ID NO: 81767.

The probes having the nucleotide sequence represented by SEQ ID NOS: 154745, 154746, 154747, 154748, 154749, 154750, 154751, 154752, 154753, 154754, 154755 were selected from the ORFs having the nucleotide sequence represented by SEQ ID NO: 81768.

The probes having the nucleotide sequence represented by SEQ ID NOS: 154756, 154757, 154758, 154759, 154760, 154761, 154762, 154763, 154764, 154765, 154766 were selected from the ORFs having the nucleotide sequence represented by SEQ ID NO: 81769.

The probes having the nucleotide sequence represented by SEQ ID NOS: 154767, 154768, 154769, 154770, 154771, 154772, 154773, 154774, 154775, 154776, 154777 were selected from the ORFs having the nucleotide sequence represented by SEQ ID NO: 81770.

The probes having the nucleotide sequence represented by SEQ ID NOS: 154778, 154779, 154780, 154781, 154782, 154783, 154784, 154785, 154786, 154787, 154788 were selected from the ORFs having the nucleotide sequence represented by SEQ ID NO: 81771.

The probes having the nucleotide sequence represented by SEQ ID NOS: 154789, 154790, 154791, 154792, 154793, 154794, 154795, 154796, 154797, 154798, 154799 were selected from the ORFs having the nucleotide sequence represented by SEQ ID NO: 81772.

The probes having the nucleotide sequence represented by SEQ ID NOS: 154800, 154801, 154802, 154803, 154804, 154805, 154806, 154807, 154808, 154809, 154810 were selected from the ORFs having the nucleotide sequence represented by SEQ ID NO: 81773.

The probes having the nucleotide sequence represented by SEQ ID NOS: 154811, 154812, 154813, 154814, 154815, 154816, 154817, 154818, 154819, 154820, 154821 were selected from the ORFs having the nucleotide sequence represented by SEQ ID NO: 81774.

The probes having the nucleotide sequence represented by SEQ ID NOS: 154822, 154823, 154824, 154825, 154826, 154827, 154828, 154829, 154830, 154831, 154832 were selected from the ORFs having the nucleotide sequence represented by SEQ ID NO: 81775.

The probes having the nucleotide sequence represented by SEQ ID NOS: 154833, 154834, 154835, 154836, 154837, 154838, 154839, 154840, 154841, 154842, 154843 were selected from the ORFs having the nucleotide sequence represented by SEQ ID NO: 81776.

The probes having the nucleotide sequence represented by SEQ ID NOS: 154844, 154845, 154846, 154847, 154848, 154849, 154850, 154851, 154852, 154853, 154854 were selected from the ORFs having the nucleotide sequence represented by SEQ ID NO: 81777.

The probes having the nucleotide sequence represented by SEQ ID NOS: 154855, 154856, 154857, 154858, 154859, 154860, 154861, 154862, 154863, 154864, 154865 were selected from the ORFs having the nucleotide sequence represented by SEQ ID NO: 81778.

The probes having the nucleotide sequence represented by SEQ ID NOS: 154866, 154867, 154868, 154869, 154870, 154871, 154872, 154873, 154874, 154875, 154876 were selected from the ORFs having the nucleotide sequence represented by SEQ ID NO: 81779.

The probes having the nucleotide sequence represented by SEQ ID NOS: 154877, 154878, 154879, 154880, 154881, 154882, 154883, 154884, 154885, 154886, 154887 were selected from the ORFs having the nucleotide sequence represented by SEQ ID NO: 81780.

The probes having the nucleotide sequence represented by SEQ ID NOS: 154888, 154889, 154890, 154891, 154892, 154893, 154894, 154895, 154896, 154897, 154898 were selected from the ORFs having the nucleotide sequence represented by SEQ ID NO: 81781.

The probes having the nucleotide sequence represented by SEQ ID NOS: 154899, 154900, 154901, 154902, 154903, 154904, 154905, 154906, 154907, 154908, 154909 were selected from the ORFs having the nucleotide sequence represented by SEQ ID NO: 81782.

The probes having the nucleotide sequence represented by SEQ ID NOS: 154910, 154911, 154912, 154913, 154914, 154915, 154916, 154917, 154918, 154919, 154920 were selected from the ORFs having the nucleotide sequence represented by SEQ ID NO: 81783.

The probes having the nucleotide sequence represented by SEQ ID NOS: 154921, 154922, 154923, 154924, 154925, 154926, 154927, 154928, 154929, 154930, 154931 were selected from the ORFs having the nucleotide sequence represented by SEQ ID NO: 81784.

The probes having the nucleotide sequence represented by SEQ ID NOS: 154932, 154933, 154934, 154935, 154936, 154937, 154938, 154939, 154940, 154941, 154942 were selected from the ORFs having the nucleotide sequence represented by SEQ ID NO: 81785.

The probes having the nucleotide sequence represented by SEQ ID NOS: 154943, 154944, 154945, 154946, 154947, 154948, 154949, 154950, 154951, 154952, 154953 were selected from the ORFs having the nucleotide sequence represented by SEQ ID NO: 81786.

The probes having the nucleotide sequence represented by SEQ ID NOS: 154954, 154955, 154956, 154957, 154958, 154959, 154960, 154961, 154962, 154963, 154964 were selected from the ORFs having the nucleotide sequence represented by SEQ ID NO: 81787.

The probes having the nucleotide sequence represented by SEQ ID NOS: 154965, 154966, 154967, 154968, 154969, 154970, 154971, 154972, 154973, 154974, 154975 were selected from the ORFs having the nucleotide sequence represented by SEQ ID NO: 81788.

The probes having the nucleotide sequence represented by SEQ ID NOS: 154976, 154977, 154978, 154979, 154980, 154981, 154982, 154983, 154984, 154985, 154986 were selected from the ORFs having the nucleotide sequence represented by SEQ ID NO: 81789.

The probes having the nucleotide sequence represented by SEQ ID NOS: 154987, 154988, 154989, 154990, 154991, 154992, 154993, 154994, 154995, 154996, 154997 were selected from the ORFs having the nucleotide sequence represented by SEQ ID NO: 81790.

The probes having the nucleotide sequence represented by SEQ ID NOS: 154998, 154999, 155000, 155001, 155002, 155003, 155004, 155005, 155006, 155007, 155008 were selected from the ORFs having the nucleotide sequence represented by SEQ ID NO: 81791.

The probes having the nucleotide sequence represented by SEQ ID NOS: 155009, 155010, 155011, 155012, 155013, 155014, 155015, 155016, 155017, 155018, 155019 were selected from the ORFs having the nucleotide sequence represented by SEQ ID NO: 81792.

The probes having the nucleotide sequence represented by SEQ ID NOS: 155020, 155021, 155022, 155023, 155024, 155025, 155026, 155027, 155028, 155029, 155030 were selected from the ORFs having the nucleotide sequence represented by SEQ ID NO: 81793.

The probes having the nucleotide sequence represented by SEQ ID NOS: 155031, 155032, 155033, 155034, 155035, 155036, 155037, 155038, 155039, 155040, 155041 were selected from the ORFs having the nucleotide sequence represented by SEQ ID NO: 81794.

The probes having the nucleotide sequence represented by SEQ ID NOS: 155042, 155043, 155044, 155045, 155046, 155047, 155048, 155049, 155050, 155051, 155052 were selected from the ORFs having the nucleotide sequence represented by SEQ ID NO: 81795.

The probes having the nucleotide sequence represented by SEQ ID NOS: 155053, 155054, 155055, 155056, 155057, 155058, 155059, 155060, 155061, 155062, 155063 were selected from the ORFs having the nucleotide sequence represented by SEQ ID NO: 81796.

The probes having the nucleotide sequence represented by SEQ ID NOS: 155064, 155065, 155066, 155067, 155068, 155069, 155070, 155071, 155072, 155073, 155074 were selected from the ORFs having the nucleotide sequence represented by SEQ ID NO: 81797.

The probes having the nucleotide sequence represented by SEQ ID NOS: 155075, 155076, 155077, 155078, 155079, 155080, 155081, 155082, 155083, 155084, 155085 were selected from the ORFs having the nucleotide sequence represented by SEQ ID NO: 81798.

The probes having the nucleotide sequence represented by SEQ ID NOS: 155086, 155087, 155088, 155089, 155090, 155091, 155092, 155093, 155094, 155095, 155096 were selected from the ORFs having the nucleotide sequence represented by SEQ ID NO: 81799.

The probes having the nucleotide sequence represented by SEQ ID NOS: 155097, 155098, 155099, 155100, 155101, 155102, 155103, 155104, 155105, 155106, 155107 were selected from the ORFs having the nucleotide sequence represented by SEQ ID NO: 81800.

The probes having the nucleotide sequence represented by SEQ ID NOS: 155108, 155109, 155110, 155111, 155112, 155113, 155114, 155115, 155116, 155117, 155118 were selected from the ORFs having the nucleotide sequence represented by SEQ ID NO: 81801.

The probes having the nucleotide sequence represented by SEQ ID NOS: 155119, 155120, 155121, 155122, 155123, 155124, 155125, 155126, 155127, 155128, 155129 were selected from the ORFs having the nucleotide sequence represented by SEQ ID NO: 81802.

The probes having the nucleotide sequence represented by SEQ ID NOS: 155130, 155131, 155132, 155133, 155134, 155135, 155136, 155137, 155138, 155139, 155140 were selected from the ORFs having the nucleotide sequence represented by SEQ ID NO: 81803.

The probes having the nucleotide sequence represented by SEQ ID NOS: 155141, 155142, 155143, 155144, 155145, 155146, 155147, 155148, 155149, 155150, 155151 were selected from the ORFs having the nucleotide sequence represented by SEQ ID NO: 81804.

The probes having the nucleotide sequence represented by SEQ ID NOS: 155152, 155153, 155154, 155155, 155156, 155157, 155158, 155159, 155160, 155161, 155162 were selected from the ORFs having the nucleotide sequence represented by SEQ ID NO: 81805.

The probes having the nucleotide sequence represented by SEQ ID NOS: 155163, 155164, 155165, 155166, 155167, 155168, 155169, 155170, 155171, 155172, 155173 were selected from the ORFs having the nucleotide sequence represented by SEQ ID NO: 81806.

The probes having the nucleotide sequence represented by SEQ ID NOS: 155174, 155175, 155176, 155177, 155178, 155179, 155180, 155181, 155182, 155183, 155184 were selected from the ORFs having the nucleotide sequence represented by SEQ ID NO: 81807.

The probes having the nucleotide sequence represented by SEQ ID NOS: 155185, 155186, 155187, 155188, 155189, 155190, 155191, 155192, 155193, 155194, 155195 were selected from the ORFs having the nucleotide sequence represented by SEQ ID NO: 81808.

The probes having the nucleotide sequence represented by SEQ ID NOS: 155196, 155197, 155198, 155199, 155200, 155201, 155202, 155203, 155204, 155205, 155206 were selected from the ORFs having the nucleotide sequence represented by SEQ ID NO: 81809.

The probes having the nucleotide sequence represented by SEQ ID NOS: 155207, 155208, 155209, 155210, 155211, 155212, 155213, 155214, 155215, 155216, 155217 were selected from the ORFs having the nucleotide sequence represented by SEQ ID NO: 81810.

The probes having the nucleotide sequence represented by SEQ ID NOS: 155218, 155219, 155220, 155221, 155222, 155223, 155224, 155225, 155226, 155227, 155228 were selected from the ORFs having the nucleotide sequence represented by SEQ ID NO: 81811.

The probes having the nucleotide sequence represented by SEQ ID NOS: 155229, 155230, 155231, 155232, 155233, 155234, 155235, 155236, 155237, 155238, 155239 were selected from the ORFs having the nucleotide sequence represented by SEQ ID NO: 81812.

The probes having the nucleotide sequence represented by SEQ ID NOS: 155240, 155241, 155242, 155243, 155244, 155245, 155246, 155247, 155248, 155249, 155250 were selected from the ORFs having the nucleotide sequence represented by SEQ ID NO: 81813.

The probes having the nucleotide sequence represented by SEQ ID NOS: 155251, 155252, 155253, 155254, 155255, 155256, 155257, 155258, 155259, 155260, 155261 were selected from the ORFs having the nucleotide sequence represented by SEQ ID NO: 81814.

The probes having the nucleotide sequence represented by SEQ ID NOS: 155262, 155263, 155264, 155265, 155266, 155267, 155268, 155269, 155270, 155271, 155272 were selected from the ORFs having the nucleotide sequence represented by SEQ ID NO: 81815.

The probes having the nucleotide sequence represented by SEQ ID NOS: 155273, 155274, 155275, 155276, 155277, 155278, 155279, 155280, 155281, 155282, 155283 were selected from the ORFs having the nucleotide sequence represented by SEQ ID NO: 81816.

The probes having the nucleotide sequence represented by SEQ ID NOS: 155284, 155285, 155286, 155287, 155288, 155289, 155290, 155291, 155292, 155293, 155294 were selected from the ORFs having the nucleotide sequence represented by SEQ ID NO: 81817.

The probes having the nucleotide sequence represented by SEQ ID NOS: 155295, 155296, 155297, 155298, 155299, 155300, 155301, 155302, 155303, 155304, 155305 were selected from the ORFs having the nucleotide sequence represented by SEQ ID NO: 81818.

The probes having the nucleotide sequence represented by SEQ ID NOS: 155306, 155307, 155308, 155309, 155310, 155311, 155312, 155313, 155314, 155315, 155316 were selected from the ORFs having the nucleotide sequence represented by SEQ ID NO: 81819.

The probes having the nucleotide sequence represented by SEQ ID NOS: 155317, 155318, 155319, 155320, 155321, 155322, 155323, 155324, 155325, 155326, 155327 were selected from the ORFs having the nucleotide sequence represented by SEQ ID NO: 81820.

The probes having the nucleotide sequence represented by SEQ ID NOS: 155328, 155329, 155330, 155331, 155332, 155333, 155334, 155335, 155336, 155337, 155338 were selected from the ORFs having the nucleotide sequence represented by SEQ ID NO: 81821.

The probes having the nucleotide sequence represented by SEQ ID NOS: 155339, 155340, 155341, 155342, 155343, 155344, 155345, 155346, 155347, 155348, 155349 were selected from the ORFs having the nucleotide sequence represented by SEQ ID NO: 81822.

The probes having the nucleotide sequence represented by SEQ ID NOS: 155350, 155351, 155352, 155353, 155354, 155355, 155356, 155357, 155358, 155359, 155360 were selected from the ORFs having the nucleotide sequence represented by SEQ ID NO: 81823.

The probes having the nucleotide sequence represented by SEQ ID NOS: 155361, 155362, 155363, 155364, 155365, 155366, 155367, 155368, 155369, 155370, 155371 were selected from the ORFs having the nucleotide sequence represented by SEQ ID NO: 81824.

The probes having the nucleotide sequence represented by SEQ ID NOS: 155372, 155373, 155374, 155375, 155376, 155377, 155378, 155379, 155380, 155381, 155382 were selected from the ORFs having the nucleotide sequence represented by SEQ ID NO: 81825.

The probes having the nucleotide sequence represented by SEQ ID NOS: 155383, 155384, 155385, 155386, 155387, 155388, 155389, 155390, 155391, 155392, 155393 were selected from the ORFs having the nucleotide sequence represented by SEQ ID NO: 81826.

The probes having the nucleotide sequence represented by SEQ ID NOS: 155394, 155395, 155396, 155397, 155398, 155399, 155400, 155401, 155402, 155403, 155404 were selected from the ORFs having the nucleotide sequence represented by SEQ ID NO: 81827.

The probes having the nucleotide sequence represented by SEQ ID NOS: 155405, 155406, 155407, 155408, 155409, 155410, 155411, 155412, 155413, 155414, 155415 were selected from the ORFs having the nucleotide sequence represented by SEQ ID NO: 81828.

The probes having the nucleotide sequence represented by SEQ ID NOS: 155416, 155417, 155418, 155419, 155420, 155421, 155422, 155423, 155424, 155425, 155426 were selected from the ORFs having the nucleotide sequence represented by SEQ ID NO: 81829.

The probes having the nucleotide sequence represented by SEQ ID NOS: 155427, 155428, 155429, 155430, 155431, 155432, 155433, 155434 were selected from the ORFs having the nucleotide sequence represented by SEQ ID NO: 81830.

The probes having the nucleotide sequence represented by SEQ ID NOS: 155435, 155436, 155437, 155438, 155439, 155440, 155441, 155442, 155443, 155444, 155445 were selected from the ORFs having the nucleotide sequence represented by SEQ ID NO: 81831.

The probes having the nucleotide sequence represented by SEQ ID NOS: 155446, 155447, 155448, 155449, 155450, 155451, 155452, 155453, 155454, 155455, 155456 were selected from the ORFs having the nucleotide sequence represented by SEQ ID NO: 81832.

The probes having the nucleotide sequence represented by SEQ ID NOS: 155457, 155458, 155459, 155460, 155461, 155462, 155463, 155464, 155465, 155466, 155467 were selected from the ORFs having the nucleotide sequence represented by SEQ ID NO: 81833.

The probes having the nucleotide sequence represented by SEQ ID NOS: 155468, 155469, 155470, 155471, 155472, 155473, 155474, 155475, 155476, 155477, 155478 were selected from the ORFs having the nucleotide sequence represented by SEQ ID NO: 81834.

The probes having the nucleotide sequence represented by SEQ ID NOS: 155479, 155480, 155481, 155482, 155483, 155484, 155485, 155486, 155487, 155488, 155489 were selected from the ORFs having the nucleotide sequence represented by SEQ ID NO: 81835.

The probes having the nucleotide sequence represented by SEQ ID NOS: 155490, 155491, 155492, 155493, 155494, 155495, 155496, 155497, 155498, 155499, 155500 were selected from the ORFs having the nucleotide sequence represented by SEQ ID NO: 81836.

The probes having the nucleotide sequence represented by SEQ ID NOS: 155501, 155502, 155503, 155504, 155505, 155506, 155507, 155508, 155509, 155510, 155511 were selected from the ORFs having the nucleotide sequence represented by SEQ ID NO: 81837.

The probes having the nucleotide sequence represented by SEQ ID NOS: 155512, 155513, 155514, 155515, 155516, 155517, 155518, 155519, 155520, 155521, 155522 were selected from the ORFs having the nucleotide sequence represented by SEQ ID NO: 81838.

The probes having the nucleotide sequence represented by SEQ ID NOS: 155523, 155524, 155525, 155526, 155527, 155528, 155529, 155530, 155531, 155532, 155533 were selected from the ORFs having the nucleotide sequence represented by SEQ ID NO: 81839.

The probes having the nucleotide sequence represented by SEQ ID NOS: 155534, 155535, 155536, 155537, 155538, 155539, 155540, 155541, 155542, 155543, 155544 were selected from the ORFs having the nucleotide sequence represented by SEQ ID NO: 81840.

The probes having the nucleotide sequence represented by SEQ ID NOS: 155545, 155546, 155547, 155548, 155549, 155550, 155551, 155552, 155553, 155554, 155555 were selected from the ORFs having the nucleotide sequence represented by SEQ ID NO: 81841.

The probes having the nucleotide sequence represented by SEQ ID NOS: 155556, 155557, 155558, 155559, 155560, 155561, 155562, 155563, 155564, 155565, 155566 were selected from the ORFs having the nucleotide sequence represented by SEQ ID NO: 81842.

The probes having the nucleotide sequence represented by SEQ ID NOS: 155567, 155568, 155569, 155570, 155571, 155572, 155573, 155574, 155575, 155576, 155577 were selected from the ORFs having the nucleotide sequence represented by SEQ ID NO: 81843.

The probes having the nucleotide sequence represented by SEQ ID NOS: 155578, 155579, 155580, 155581, 155582, 155583, 155584, 155585, 155586, 155587, 155588 were selected from the ORFs having the nucleotide sequence represented by SEQ ID NO: 81844.

The probes having the nucleotide sequence represented by SEQ ID NOS: 155589, 155590, 155591, 155592, 155593, 155594, 155595, 155596, 155597, 155598, 155599 were selected from the ORFs having the nucleotide sequence represented by SEQ ID NO: 81845.

The probes having the nucleotide sequence represented by SEQ ID NOS: 155600, 155601, 155602, 155603, 155604, 155605, 155606, 155607, 155608, 155609, 155610 were selected from the ORFs having the nucleotide sequence represented by SEQ ID NO: 81846.

The probes having the nucleotide sequence represented by SEQ ID NOS: 155611, 155612, 155613, 155614, 155615, 155616, 155617, 155618, 155619, 155620, 155621 were selected from the ORFs having the nucleotide sequence represented by SEQ ID NO: 81847.

The probes having the nucleotide sequence represented by SEQ ID NOS: 155622, 155623, 155624, 155625, 155626, 155627, 155628, 155629, 155630, 155631, 155632 were selected from the ORFs having the nucleotide sequence represented by SEQ ID NO: 81848.

The probes having the nucleotide sequence represented by SEQ ID NOS: 155633, 155634, 155635, 155636, 155637, 155638, 155639, 155640, 155641, 155642, 155643 were selected from the ORFs having the nucleotide sequence represented by SEQ ID NO: 81849.

The probes having the nucleotide sequence represented by SEQ ID NOS: 155644, 155645, 155646, 155647, 155648, 155649, 155650, 155651, 155652, 155653, 155654 were selected from the ORFs having the nucleotide sequence represented by SEQ ID NO: 81850.

The probes having the nucleotide sequence represented by SEQ ID NOS: 155655, 155656, 155657, 155658, 155659, 155660, 155661, 155662, 155663, 155664, 155665 were selected from the ORFs having the nucleotide sequence represented by SEQ ID NO: 81851.

The probes having the nucleotide sequence represented by SEQ ID NOS: 155666, 155667, 155668, 155669, 155670, 155671, 155672, 155673, 155674, 155675, 155676 were selected from the ORFs having the nucleotide sequence represented by SEQ ID NO: 81852.

The probes having the nucleotide sequence represented by SEQ ID NOS: 155677, 155678, 155679, 155680, 155681, 155682, 155683, 155684, 155685, 155686, 155687 were selected from the ORFs having the nucleotide sequence represented by SEQ ID NO: 81853.

The probes having the nucleotide sequence represented by SEQ ID NOS: 155688, 155689, 155690, 155691, 155692, 155693, 155694, 155695, 155696, 155697, 155698 were selected from the ORFs having the nucleotide sequence represented by SEQ ID NO: 81854.

The probes having the nucleotide sequence represented by SEQ ID NOS: 155699, 155700, 155701, 155702, 155703, 155704, 155705, 155706, 155707, 155708, 155709 were selected from the ORFs having the nucleotide sequence represented by SEQ ID NO: 81855.

The probes having the nucleotide sequence represented by SEQ ID NOS: 155710, 155711, 155712, 155713, 155714, 155715, 155716, 155717, 155718, 155719, 155720 were selected from the ORFs having the nucleotide sequence represented by SEQ ID NO: 81856.

The probes having the nucleotide sequence represented by SEQ ID NOS: 155721, 155722, 155723, 155724, 155725, 155726, 155727, 155728, 155729, 155730, 155731 were selected from the ORFs having the nucleotide sequence represented by SEQ ID NO: 81857.

The probes having the nucleotide sequence represented by SEQ ID NOS: 155732, 155733, 155734, 155735, 155736, 155737, 155738, 155739, 155740, 155741, 155742 were selected from the ORFs having the nucleotide sequence represented by SEQ ID NO: 81858.

The probes having the nucleotide sequence represented by SEQ ID NOS: 155743, 155744, 155745, 155746, 155747, 155748, 155749, 155750, 155751, 155752, 155753 were selected from the ORFs having the nucleotide sequence represented by SEQ ID NO: 81859.

The probes having the nucleotide sequence represented by SEQ ID NOS: 155754, 155755, 155756, 155757, 155758, 155759, 155760, 155761, 155762, 155763, 155764 were selected from the ORFs having the nucleotide sequence represented by SEQ ID NO: 81860.

The probes having the nucleotide sequence represented by SEQ ID NOS: 155765, 155766, 155767, 155768, 155769, 155770, 155771, 155772, 155773, 155774, 155775 were selected from the ORFs having the nucleotide sequence represented by SEQ ID NO: 81861.

The probes having the nucleotide sequence represented by SEQ ID NOS: 155776, 155777, 155778, 155779, 155780, 155781, 155782, 155783, 155784, 155785, 155786 were selected from the ORFs having the nucleotide sequence represented by SEQ ID NO: 81862.

The probes having the nucleotide sequence represented by SEQ ID NOS: 155787, 155788, 155789, 155790, 155791, 155792, 155793, 155794, 155795, 155796, 155797 were selected from the ORFs having the nucleotide sequence represented by SEQ ID NO: 81863.

The probes having the nucleotide sequence represented by SEQ ID NOS: 155798, 155799, 155800, 155801, 155802, 155803, 155804, 155805, 155806, 155807, 155808 were selected from the ORFs having the nucleotide sequence represented by SEQ ID NO: 81864.

The probes having the nucleotide sequence represented by SEQ ID NOS: 155809, 155810, 155811, 155812, 155813, 155814, 155815, 155816, 155817, 155818, 155819 were selected from the ORFs having the nucleotide sequence represented by SEQ ID NO: 81865.

The probes having the nucleotide sequence represented by SEQ ID NOS: 155820, 155821, 155822, 155823, 155824, 155825, 155826, 155827, 155828, 155829, 155830 were selected from the ORFs having the nucleotide sequence represented by SEQ ID NO: 81866.

The probes having the nucleotide sequence represented by SEQ ID NOS: 155831, 155832, 155833, 155834, 155835, 155836, 155837, 155838, 155839, 155840, 155841 were selected from the ORFs having the nucleotide sequence represented by SEQ ID NO: 81867.

The probes having the nucleotide sequence represented by SEQ ID NOS: 155842, 155843, 155844, 155845, 155846, 155847, 155848, 155849, 155850, 155851, 155852 were selected from the ORFs having the nucleotide sequence represented by SEQ ID NO: 81868.

The probes having the nucleotide sequence represented by SEQ ID NOS: 155853, 155854, 155855, 155856, 155857, 155858, 155859, 155860, 155861, 155862, 155863 were selected from the ORFs having the nucleotide sequence represented by SEQ ID NO: 81869.

The probes having the nucleotide sequence represented by SEQ ID NOS: 155864, 155865, 155866, 155867, 155868, 155869, 155870, 155871, 155872, 155873, 155874 were selected from the ORFs having the nucleotide sequence represented by SEQ ID NO: 81870.

The probes having the nucleotide sequence represented by SEQ ID NOS: 155875, 155876, 155877, 155878, 155879, 155880, 155881, 155882, 155883, 155884, 155885 were selected from the ORFs having the nucleotide sequence represented by SEQ ID NO: 81871.

The probes having the nucleotide sequence represented by SEQ ID NOS: 155886, 155887, 155888, 155889, 155890, 155891, 155892, 155893, 155894, 155895, 155896 were selected from the ORFs having the nucleotide sequence represented by SEQ ID NO: 81872.

The probes having the nucleotide sequence represented by SEQ ID NOS: 155897, 155898, 155899, 155900, 155901, 155902, 155903, 155904, 155905, 155906, 155907 were selected from the ORFs having the nucleotide sequence represented by SEQ ID NO: 81873.

The probes having the nucleotide sequence represented by SEQ ID NOS: 155908, 155909, 155910, 155911, 155912, 155913, 155914, 155915, 155916, 155917, 155918 were selected from the ORFs having the nucleotide sequence represented by SEQ ID NO: 81874.

The probes having the nucleotide sequence represented by SEQ ID NOS: 155919, 155920, 155921, 155922, 155923, 155924, 155925, 155926, 155927, 155928, 155929 were selected from the ORFs having the nucleotide sequence represented by SEQ ID NO: 81875.

The probes having the nucleotide sequence represented by SEQ ID NOS: 155930, 155931, 155932, 155933, 155934, 155935, 155936, 155937, 155938, 155939, 155940 were selected from the ORFs having the nucleotide sequence represented by SEQ ID NO: 81876.

The probes having the nucleotide sequence represented by SEQ ID NOS: 155941, 155942, 155943, 155944, 155945, 155946, 155947, 155948, 155949, 155950, 155951 were selected from the ORFs having the nucleotide sequence represented by SEQ ID NO: 81877.

The probes having the nucleotide sequence represented by SEQ ID NOS: 155952, 155953, 155954, 155955, 155956, 155957, 155958, 155959, 155960, 155961, 155962 were selected from the ORFs having the nucleotide sequence represented by SEQ ID NO: 81878.

The probes having the nucleotide sequence represented by SEQ ID NOS: 155963, 155964, 155965, 155966, 155967, 155968, 155969, 155970, 155971, 155972, 155973 were selected from the ORFs having the nucleotide sequence represented by SEQ ID NO: 81879.

The probes having the nucleotide sequence represented by SEQ ID NOS: 155974, 155975, 155976, 155977, 155978, 155979, 155980, 155981, 155982, 155983, 155984 were selected from the ORFs having the nucleotide sequence represented by SEQ ID NO: 81880.

The probes having the nucleotide sequence represented by SEQ ID NOS: 155985, 155986, 155987, 155988, 155989, 155990, 155991, 155992, 155993, 155994, 155995 were selected from the ORFs having the nucleotide sequence represented by SEQ ID NO: 81881.

The probes having the nucleotide sequence represented by SEQ ID NOS: 155996, 155997, 155998, 155999, 156000, 156001, 156002, 156003, 156004, 156005, 156006 were selected from the ORFs having the nucleotide sequence represented by SEQ ID NO: 81882.

The probes having the nucleotide sequence represented by SEQ ID NOS: 156007, 156008, 156009, 156010, 156011, 156012, 156013, 156014, 156015, 156016, 156017 were selected from the ORFs having the nucleotide sequence represented by SEQ ID NO: 81883.

The probes having the nucleotide sequence represented by SEQ ID NOS: 156018, 156019, 156020, 156021, 156022, 156023, 156024, 156025, 156026, 156027, 156028 were selected from the ORFs having the nucleotide sequence represented by SEQ ID NO: 81884.

The probes having the nucleotide sequence represented by SEQ ID NOS: 156029, 156030, 156031, 156032, 156033, 156034, 156035, 156036, 156037, 156038, 156039 were selected from the ORFs having the nucleotide sequence represented by SEQ ID NO: 81885.

The probes having the nucleotide sequence represented by SEQ ID NOS: 156040, 156041, 156042, 156043, 156044, 156045, 156046, 156047, 156048, 156049, 156050 were selected from the ORFs having the nucleotide sequence represented by SEQ ID NO: 81886.

The probes having the nucleotide sequence represented by SEQ ID NOS: 156051, 156052, 156053, 156054, 156055, 156056, 156057, 156058, 156059, 156060, 156061 were selected from the ORFs having the nucleotide sequence represented by SEQ ID NO: 81887.

The probes having the nucleotide sequence represented by SEQ ID NOS: 156062, 156063, 156064, 156065, 156066, 156067, 156068, 156069, 156070, 156071, 156072 were selected from the ORFs having the nucleotide sequence represented by SEQ ID NO: 81888.

The probes having the nucleotide sequence represented by SEQ ID NOS: 156073, 156074, 156075, 156076, 156077, 156078, 156079, 156080, 156081, 156082, 156083 were selected from the ORFs having the nucleotide sequence represented by SEQ ID NO: 81889.

The probes having the nucleotide sequence represented by SEQ ID NOS: 156084, 156085, 156086, 156087, 156088, 156089, 156090, 156091, 156092, 156093, 156094 were selected from the ORFs having the nucleotide sequence represented by SEQ ID NO: 81890.

The probes having the nucleotide sequence represented by SEQ ID NOS: 156095, 156096, 156097, 156098, 156099, 156100, 156101, 156102, 156103, 156104, 156105 were selected from the ORFs having the nucleotide sequence represented by SEQ ID NO: 81891.

The probes having the nucleotide sequence represented by SEQ ID NOS: 156106, 156107, 156108, 156109, 156110, 156111, 156112, 156113, 156114, 156115, 156116 were selected from the ORFs having the nucleotide sequence represented by SEQ ID NO: 81892.

The probes having the nucleotide sequence represented by SEQ ID NOS: 156117, 156118, 156119, 156120, 156121, 156122, 156123, 156124, 156125, 156126, 156127 were selected from the ORFs having the nucleotide sequence represented by SEQ ID NO: 81893.

The probes having the nucleotide sequence represented by SEQ ID NOS: 156128, 156129, 156130, 156131, 156132, 156133, 156134, 156135, 156136, 156137, 156138, 156139, 156140, 156141, 156142, 156143, 156144, 156145, 156146, 156147, 156148, 156149 were selected from the ORFs having the nucleotide sequence represented by SEQ ID NO: 81894.

The probes having the nucleotide sequence represented by SEQ ID NOS: 156150, 156151, 156152, 156153, 156154, 156155, 156156, 156157, 156158, 156159, 156160 were selected from the ORFs having the nucleotide sequence represented by SEQ ID NO: 81895.

The probes having the nucleotide sequence represented by SEQ ID NOS: 156161, 156162, 156163, 156164, 156165, 156166, 156167, 156168, 156169, 156170, 156171 were selected from the ORFs having the nucleotide sequence represented by SEQ ID NO: 81896.

The probes having the nucleotide sequence represented by SEQ ID NOS: 156172, 156173, 156174, 156175, 156176, 156177, 56178, 156179, 156180, 156181, 156182 were selected from the ORFs having the nucleotide sequence represented by SEQ ID NO: 81897.

The probes having the nucleotide sequence represented by SEQ ID NOS: 156183, 156184, 156185, 156186, 156187, 156188, 156189, 156190, 156191, 156192, 156193 were selected from the ORFs having the nucleotide sequence represented by SEQ ID NO: 81898.

The probes having the nucleotide sequence represented by SEQ ID NOS: 156194, 156195, 156196, 156197, 156198, 156199, 156200, 156201, 156202, 156203, 156204 were selected from the ORFs having the nucleotide sequence represented by SEQ ID NOS: 81899.

The probes having the nucleotide sequence represented by SEQ ID NOS: 156205, 156206, 156207, 156208, 156209, 156210, 156211, 156212, 156213, 156214, 156215 were selected from the ORFs having the nucleotide sequence represented by SEQ ID NO: 81900.

The probes having the nucleotide sequence represented by SEQ ID NOS: 156216, 156217, 156218, 156219, 156220, 156221, 156222, 156223, 156224, 156225, 156226 were selected from the ORFs having the nucleotide sequence represented by SEQ ID NO: 81901.

The probes having the nucleotide sequence represented by SEQ ID NOS: 156227, 156228, 156229, 156230, 156231, 156232, 156233, 156234, 156235, 156236, 156237 were selected from the ORFs having the nucleotide sequence represented by SEQ ID NO: 81902.

The probes having the nucleotide sequence represented by SEQ ID NOS: 156238, 156239, 156240, 156241, 156242, 156243, 156244, 156245, 156246, 156247, 156248 were selected from the ORFs having the nucleotide sequence represented by SEQ ID NO: 81903.

The probes having the nucleotide sequence represented by SEQ ID NOS: 156249, 156250, 156251, 156252, 156253, 156254, 156255, 156256, 156257, 156258, 156259 were selected from the ORFs having the nucleotide sequence represented by SEQ ID NO: 81904.

The probes having the nucleotide sequence represented by SEQ ID NOS: 156260, 156261, 156262, 156263, 156264, 156265, 156266, 156267, 156268, 156269, 156270 were selected from the ORFs having the nucleotide sequence represented by SEQ ID NO: 81905.

The probes having the nucleotide sequence represented by SEQ ID NOS: 156271, 156272, 156273, 156274, 156275, 156276, 156277, 156278, 156279, 156280, 156281 were selected from the ORFs having the nucleotide sequence represented by SEQ ID NO: 81906.

The probes having the nucleotide sequence represented by SEQ ID NOS: 156282, 156283, 156284, 156285, 156286, 156287, 156288, 156289, 156290, 156291, 156292 were selected from the ORFs having the nucleotide sequence represented by SEQ ID NO: 81907.

The probes having the nucleotide sequence represented by SEQ ID NOS: 156293, 156294, 156295, 156296, 156297, 156298, 156299, 156300, 156301, 156302, 156303 were selected from the ORFs having the nucleotide sequence represented by SEQ ID NO: 81908.

The probes having the nucleotide sequence represented by SEQ ID NOS: 156304, 156305, 156306, 156307, 156308, 156309, 156310, 156311, 156312, 156313, 156314 were selected from the ORFs having the nucleotide sequence represented by SEQ ID NO: 81909.

The probes having the nucleotide sequence represented by SEQ ID NOS: 156315, 156316, 156317, 156318, 156319, 156320, 156321, 156322, 156323, 156324, 156325 were selected from the ORFs having the nucleotide sequence represented by SEQ ID NO: 81910.

The probes having the nucleotide sequence represented by SEQ ID NOS: 156326, 156327, 156328, 156329, 156330, 156331, 156332, 156333, 156334, 156335, 156336 were selected from the ORFs having the nucleotide sequence represented by SEQ ID NO: 81911.

The probes having the nucleotide sequence represented by SEQ ID NOS: 156337, 156338, 156339, 156340, 156341, 156342, 156343, 156344, 156345, 156346, 156347 were selected from the ORFs having the nucleotide sequence represented by SEQ ID NO: 81912.

The probes having the nucleotide sequence represented by SEQ ID NOS: 156348, 156349, 156350, 156351, 156352, 156353, 156354, 156355, 156356, 156357, 156358 were selected from the ORFs having the nucleotide sequence represented by SEQ ID NO: 81913.

The probes having the nucleotide sequence represented by SEQ ID NOS: 156359, 156360, 156361, 156362, 156363, 156364, 156365, 156366, 156367, 156368, 156369, 156370, 156371, 156372, 156373, 156374, 156375, 156376, 156377, 156378, 156379, 156380 were selected from the ORFs having the nucleotide sequence represented by SEQ ID NO: 81914.

The probes having the nucleotide sequence represented by SEQ ID NOS: 156381, 156382, 156383, 156384, 156385, 156386, 156387, 156388, 156389, 156390, 156391 were selected from the ORFs having the nucleotide sequence represented by SEQ ID NO: 81915.

The probes having the nucleotide sequence represented by SEQ ID NOS: 156392, 156393, 156394, 156395, 156396, 156397, 156398, 156399, 156400, 156401, 156402 were selected from the ORFs having the nucleotide sequence represented by SEQ ID NO: 81916.

The probes having the nucleotide sequence represented by SEQ ID NOS: 156403, 156404, 156405, 156406, 156407, 156408, 156409, 156410, 156411, 156412, 156413 were selected from the ORFs having the nucleotide sequence represented by SEQ ID NO: 81917.

The probes having the nucleotide sequence represented by SEQ ID NOS: 156414, 156415, 156416, 156417, 156418, 156419, 156420, 156421, 156422, 156423, 156424 were selected from the ORFs having the nucleotide sequence represented by SEQ ID NO: 81918.

The probes having the nucleotide sequence represented by SEQ ID NOS: 156425, 156426, 156427, 156428, 156429, 156430, 156431, 156432, 156433, 156434, 156435 were selected from the ORFs having the nucleotide sequence represented by SEQ ID NO: 81919.

The probes having the nucleotide sequence represented by SEQ ID NOS: 156436, 156437, 156438, 156439, 156440, 156441, 156442, 156443, 156444, 156445, 156446 were selected from the ORFs having the nucleotide sequence represented by SEQ ID NO: 81920.

The probes having the nucleotide sequence represented by SEQ ID NOS: 156447, 156448, 156449, 156450, 156451, 156452, 156453, 156454, 156455, 156456, 156457 were selected from the ORFs having the nucleotide sequence represented by SEQ ID NO: 81921.

The probes having the nucleotide sequence represented by SEQ ID NOS: 156458, 156459, 156460, 156461, 156462, 156463, 156464, 156465, 156466, 156467, 156468 were selected from the ORFs having the nucleotide sequence represented by SEQ ID NO: 81922.

The probes having the nucleotide sequence represented by SEQ ID NOS: 156469, 156470, 156471, 156472, 156473, 156474, 156475, 156476, 156477, 156478, 156479 were selected from the ORFs having the nucleotide sequence represented by SEQ ID NO: 81923.

The probes having the nucleotide sequence represented by SEQ ID NOS: 156480, 156481, 156482, 156483, 156484, 156485, 156486, 156487, 156488, 156489, 156490 were selected from the ORFs having the nucleotide sequence represented by SEQ ID NO: 81924.

The probes having the nucleotide sequence represented by SEQ ID NOS: 156491, 156492, 156493, 156494, 156495, 156496, 156497, 156498, 156499, 156500, 156501 were selected from the ORFs having the nucleotide sequence represented by SEQ ID NO: 81925.

The probes having the nucleotide sequence represented by SEQ ID NOS: 156502, 156503, 156504, 156505, 156506, 156507, 156508, 156509, 156510, 156511, 156512 were selected from the ORFs having the nucleotide sequence represented by SEQ ID NO: 81926.

The probes having the nucleotide sequence represented by SEQ ID NOS: 156513, 156514, 156515, 156516, 156517, 156518, 156519, 156520, 156521, 156522, 156523 were selected from the ORFs having the nucleotide sequence represented by SEQ ID NO: 81927.

The probes having the nucleotide sequence represented by SEQ ID NOS: 156524, 156525, 156526, 156527, 156528, 156529, 156530, 156531, 156532, 156533, 156534 were selected from the ORFs having the nucleotide sequence represented by SEQ ID NO: 81928.

The probes having the nucleotide sequence represented by SEQ ID NOS: 156535, 156536, 156537, 156538, 156539, 156540, 156541, 156542, 156543, 156544, 156545 were selected from the ORFs having the nucleotide sequence represented by SEQ ID NO: 81929.

The probes having the nucleotide sequence represented by SEQ ID NOS: 156546, 156547, 156548, 156549, 156550, 156551, 156552, 156553, 156554, 156555, 156556 were selected from the ORFs having the nucleotide sequence represented by SEQ ID NO: 81930.

The probes having the nucleotide sequence represented by SEQ ID NOS: 156557, 156558, 156559, 156560, 156561, 156562, 56563, 156564, 156565, 156566, 156567 were selected from the ORFs having the nucleotide sequence represented by SEQ ID NO: 81931.

The probes having the nucleotide sequence represented by SEQ ID NOS: 156568, 156569, 156570, 156571, 156572, 156573, 156574, 156575, 156576, 156577, 156578 were selected from the ORFs having the nucleotide sequence represented by SEQ ID NO: 81932.

The probes having the nucleotide sequence represented by SEQ ID NOS: 156579, 156580, 156581, 156582, 156583, 156584, 156585, 156586, 156587, 156588, 156589 were selected from the ORFs having the nucleotide sequence represented by SEQ ID NO: 81933.

The probes having the nucleotide sequence represented by SEQ ID NOS: 156590, 156591, 156592, 156593, 156594, 156595, 156596, 156597, 156598, 156599, 156600 were selected from the ORFs having the nucleotide sequence represented by SEQ ID NO: 81934.

The probes having the nucleotide sequence represented by SEQ ID NOS: 156601, 156602, 156603, 156604, 156605, 156606, 156607, 156608, 156609, 156610, 156611 were selected from the ORFs having the nucleotide sequence represented by SEQ ID NO: 81935.

The probes having the nucleotide sequence represented by SEQ ID NOS: 156612, 156613, 156614, 156615, 156616, 156617, 156618, 156619, 156620, 156621, 156622 were selected from the ORFs having the nucleotide sequence represented by SEQ ID NO: 81936.

The probes having the nucleotide sequence represented by SEQ ID NOS: 156623, 156624, 156625, 156626, 156627, 156628, 156629, 156630, 156631, 156632, 156633 were selected from the ORFs having the nucleotide sequence represented by SEQ ID NO: 81937.

The probes having the nucleotide sequence represented by SEQ ID NOS: 156634, 156635, 156636, 156637, 156638, 156639, 156640, 156641, 156642, 156643, 156644 were selected from the ORFs having the nucleotide sequence represented by SEQ ID NO: 81938.

The probes having the nucleotide sequence represented by SEQ ID NOS: 156645, 156646, 156647, 156648, 156649, 156650, 156651, 156652, 156653, 156654, 156655 were selected from the ORFs having the nucleotide sequence represented by SEQ ID NO: 81939.

The probes having the nucleotide sequence represented by SEQ ID NOS: 156656, 156657, 156658, 156659, 156660, 156661, 156662, 156663, 156664, 156665, 156666 were selected from the ORFs having the nucleotide sequence represented by SEQ ID NO: 81940.

The probes having the nucleotide sequence represented by SEQ ID NOS: 156667, 156668, 156669, 156670, 156671, 156672, 156673, 156674, 156675, 156676, 156677 were selected from the ORFs having the nucleotide sequence represented by SEQ ID NO: 81941.

The probes having the nucleotide sequence represented by SEQ ID NOS: 156678, 156679, 156680, 156681, 156682, 156683, 156684, 156685, 156686, 156687, 156688 were selected from the ORFs having the nucleotide sequence represented by SEQ ID NO: 81942.

The probes having the nucleotide sequence represented by SEQ ID NOS: 156689, 156690, 156691, 156692, 156693, 156694, 156695, 156696, 156697, 156698, 156699 were selected from the ORFs having the nucleotide sequence represented by SEQ ID NO: 81943.

The probes having the nucleotide sequence represented by SEQ ID NOS: 156700, 156701, 156702, 156703, 156704, 156705, 156706, 156707, 156708, 156709, 156710 were selected from the ORFs having the nucleotide sequence represented by SEQ ID NO: 81944.

The probes having the nucleotide sequence represented by SEQ ID NOS: 156711, 156712, 156713, 156714, 156715, 156716, 156717, 156718, 156719, 156720, 156721 were selected from the ORFs having the nucleotide sequence represented by SEQ ID NO: 81945.

The probes having the nucleotide sequence represented by SEQ ID NOS: 156722, 156723, 156724, 156725, 156726, 156727, 156728, 156729, 156730, 156731, 156732 were selected from the ORFs having the nucleotide sequence represented by SEQ ID NO: 81946.

The probes having the nucleotide sequence represented by SEQ ID NOS: 156733, 156734, 156735, 156736, 156737, 156738, 156739, 156740, 156741, 156742, 156743 were selected from the ORFs having the nucleotide sequence represented by SEQ ID NO: 81947.

The probes having the nucleotide sequence represented by SEQ ID NOS: 156744, 156745, 156746, 156747, 156748, 156749, 156750, 156751, 156752, 156753, 156754 were selected from the ORFs having the nucleotide sequence represented by SEQ ID NO: 81948.

The probes having the nucleotide sequence represented by SEQ ID NOS: 156755, 156756, 156757, 156758, 156759, 156760, 156761, 156762, 156763, 156764, 156765 were selected from the ORFs having the nucleotide sequence represented by SEQ ID NO: 81949.

The probes having the nucleotide sequence represented by SEQ ID NOS: 156766, 156767, 156768, 156769, 156770, 156771, 156772, 156773, 156774, 156775, 156776 were selected from the ORFs having the nucleotide sequence represented by SEQ ID NO: 81950.

The probes having the nucleotide sequence represented by SEQ ID NOS: 156777, 156778, 156779, 156780, 156781, 156782, 156783, 156784, 156785, 156786, 156787 were selected from the ORFs having the nucleotide sequence represented by SEQ ID NO: 81951.

The probes having the nucleotide sequence represented by SEQ ID NOS: 156788, 156789, 156790, 156791, 156792, 156793, 156794, 156795, 156796, 156797, 156798 were selected from the ORFs having the nucleotide sequence represented by SEQ ID NO: 81952.

The probes having the nucleotide sequence represented by SEQ ID NOS: 156799, 156800, 156801, 156802, 156803, 156804, 156805, 156806, 156807, 156808, 156809 were selected from the ORFs having the nucleotide sequence represented by SEQ ID NO: 81953.

The probes having the nucleotide sequence represented by SEQ ID NOS: 156810, 156811, 156812, 156813, 156814, 156815, 156816, 156817, 156818, 156819, 156820 were selected from the ORFs having the nucleotide sequence represented by SEQ ID NO: 81954.

The probes having the nucleotide sequence represented by SEQ ID NOS: 156821, 156822, 156823, 156824, 156825, 156826, 156827, 156828, 156829, 156830, 156831 were selected from the ORFs having the nucleotide sequence represented by SEQ ID NO: 81955.

The probes having the nucleotide sequence represented by SEQ ID NOS: 156832, 156833, 156834, 156835, 156836, 156837, 156838, 156839, 156840, 156841, 156842 were selected from the ORFs having the nucleotide sequence represented by SEQ ID NO: 81956.

The probes having the nucleotide sequence represented by SEQ ID NOS: 156843, 156844, 156845, 156846, 156847, 156848, 156849, 156850, 156851, 156852, 156853 were selected from the ORFs having the nucleotide sequence represented by SEQ ID NO: 81957.

The probes having the nucleotide sequence represented by SEQ ID NOS: 156854, 156855, 156856, 156857, 156858, 156859, 156860, 156861, 156862, 156863, 156864 were selected from the ORFs having the nucleotide sequence represented by SEQ ID NO: 81958.

The probes having the nucleotide sequence represented by SEQ ID NOS: 156865, 156866, 156867, 156868, 156869, 156870, 156871, 156872, 156873, 156874, 156875 were selected from the ORFs having the nucleotide sequence represented by SEQ ID NO: 81959.

The probes having the nucleotide sequence represented by SEQ ID NOS: 156876, 156877, 156878, 156879, 156880, 156881, 156882, 156883, 156884, 156885, 156886 were selected from the ORFs having the nucleotide sequence represented by SEQ ID NO: 81960.

The probes having the nucleotide sequence represented by SEQ ID NOS: 156887, 156888, 156889, 156890, 156891, 156892, 156893, 156894, 156895, 156896, 156897 were selected from the ORFs having the nucleotide sequence represented by SEQ ID NO: 81961.

The probes having the nucleotide sequence represented by SEQ ID NOS: 156898, 156899, 156900, 156901, 156902, 156903, 156904, 156905, 156906, 156907, 156908 were selected from the ORFs having the nucleotide sequence represented by SEQ ID NO: 81962.

The probes having the nucleotide sequence represented by SEQ ID NOS: 156909, 156910, 156911, 156912, 156913, 156914, 156915, 156916, 156917, 156918, 156919 were selected from the ORFs having the nucleotide sequence represented by SEQ ID NO: 81963.

The probes having the nucleotide sequence represented by SEQ ID NOS: 156920, 156921, 156922, 156923, 156924, 156925, 156926, 156927, 156928, 156929, 156930 were selected from the ORFs having the nucleotide sequence represented by SEQ ID NO: 81964.

The probes having the nucleotide sequence represented by SEQ ID NOS: 156931, 156932, 156933, 156934, 156935, 156936, 156937, 156938, 156939, 156940, 156941 were selected from the ORFs having the nucleotide sequence represented by SEQ ID NO: 81965.

The probes having the nucleotide sequence represented by SEQ ID NOS: 156942, 156943, 156944, 156945, 156946, 156947, 156948, 156949, 156950, 156951, 156952 were selected from the ORFs having the nucleotide sequence represented by SEQ ID NO: 81966.

The probes having the nucleotide sequence represented by SEQ ID NOS: 156953, 156954, 156955, 156956, 156957, 156958, 156959, 156960, 156961, 156962, 156963 were selected from the ORFs having the nucleotide sequence represented by SEQ ID NO: 81967.

The probes having the nucleotide sequence represented by SEQ ID NOS: 156964, 156965, 156966, 156967, 156968, 156969, 156970, 156971, 156972, 156973, 156974 were selected from the ORFs having the nucleotide sequence represented by SEQ ID NO: 81968.

The probes having the nucleotide sequence represented by SEQ ID NOS: 156975, 156976, 156977, 156978, 156979, 156980, 156981, 156982, 156983, 156984, 156985 were selected from the ORFs having the nucleotide sequence represented by SEQ ID NO: 81969.

The probes having the nucleotide sequence represented by SEQ ID NOS: 156986, 156987, 156988, 156989, 156990, 156991, 156992, 156993, 156994, 156995, 156996 were selected from the ORFs having the nucleotide sequence represented by SEQ ID NO: 81970.

The probes having the nucleotide sequence represented by SEQ ID NOS: 156997, 156998, 156999, 157000, 157001, 157002, 157003, 157004, 157005, 157006, 157007 were selected from the ORFs having the nucleotide sequence represented by SEQ ID NO: 81971.

The probes having the nucleotide sequence represented by SEQ ID NOS: 157008, 157009, 157010, 157011, 157012, 157013, 157014, 157015, 157016, 157017, 157018 were selected from the ORFs having the nucleotide sequence represented by SEQ ID NO: 81972.

The probes having the nucleotide sequence represented by SEQ ID NOS: 157019, 157020, 157021, 157022, 157023, 157024, 157025, 157026, 157027, 157028, 157029 were selected from the ORFs having the nucleotide sequence represented by SEQ ID NO: 81973.

The probes having the nucleotide sequence represented by SEQ ID NOS: 157030, 157031, 157032, 157033, 157034, 157035, 157036, 157037, 157038, 157039, 157040 were selected from the ORFs having the nucleotide sequence represented by SEQ ID NO: 81974.

The probes having the nucleotide sequence represented by SEQ ID NOS: 157041, 157042, 157043, 157044, 157045, 157046, 157047, 157048, 157049, 157050, 157051 were selected from the ORFs having the nucleotide sequence represented by SEQ ID NO: 81975.

The probes having the nucleotide sequence represented by SEQ ID NOS: 157052, 157053, 157054, 157055, 157056, 157057, 157058, 157059 were selected from the ORFs having the nucleotide sequence represented by SEQ ID NO: 81976.

The probes having the nucleotide sequence represented by SEQ ID NOS: 157060, 157061, 157062, 157063, 157064, 157065, 157066, 157067, 157068, 157069, 157070 were selected from the ORFs having the nucleotide sequence represented by SEQ ID NO: 81977.

The probes having the nucleotide sequence represented by SEQ ID NOS: 157071, 157072, 157073, 157074, 157075, 157076, 157077, 157078, 157079, 157080, 157081 were selected from the ORFs having the nucleotide sequence represented by SEQ ID NO: 81978.

The probes having the nucleotide sequence represented by SEQ ID NOS: 157082, 157083, 157084, 157085, 157086, 157087, 157088, 157089, 157090, 157091, 157092 were selected from the ORFs having the nucleotide sequence represented by SEQ ID NO: 81979.

The probes having the nucleotide sequence represented by SEQ ID NOS: 157093, 157094, 157095, 157096, 157097, 157098, 157099, 157100, 157101, 157102, 157103 were selected from the ORFs having the nucleotide sequence represented by SEQ ID NO: 81980.

The probes having the nucleotide sequence represented by SEQ ID NOS: 157104, 157105, 157106, 157107, 157108, 157109, 157110, 157111, 157112, 157113, 157114 were selected from the ORFs having the nucleotide sequence represented by SEQ ID NO: 81981.

The probes having the nucleotide sequence represented by SEQ ID NOS: 157115, 157116, 157117, 157118, 157119, 157120, 157121, 157122, 157123, 157124, 157125 were selected from the ORFs having the nucleotide sequence represented by SEQ ID NO: 81982.

The probes having the nucleotide sequence represented by SEQ ID NOS: 157126, 157127, 157128, 157129, 157130, 157131, 157132, 157133, 157134, 157135, 157136 were selected from the ORFs having the nucleotide sequence represented by SEQ ID NO: 81983.

The probes having the nucleotide sequence represented by SEQ ID NOS: 157137, 157138, 157139, 157140, 157141, 157142, 157143, 157144, 157145, 157146, 157147 were selected from the ORFs having the nucleotide sequence represented by SEQ ID NO: 81984.

The probes having the nucleotide sequence represented by SEQ ID NOS: 157148, 157149, 157150, 157151, 157152, 157153, 157154, 157155, 157156, 157157, 157158 were selected from the ORFs having the nucleotide sequence represented by SEQ ID NO: 81985.

The probes having the nucleotide sequence represented by SEQ ID NOS: 157159, 157160, 157161, 157162, 157163, 157164, 157165, 157166, 157167, 157168, 157169 were selected from the ORFs having the nucleotide sequence represented by SEQ ID NO: 81986.

The probes having the nucleotide sequence represented by SEQ ID NOS: 157170, 157171, 157172, 157173, 157174, 157175, 157176, 157177, 157178, 157179, 157180 were selected from the ORFs having the nucleotide sequence represented by SEQ ID NO: 81987.

The probes having the nucleotide sequence represented by SEQ ID NOS: 157181, 157182, 157183, 157184, 157185, 157186, 157187, 157188, 157189, 157190, 157191 were selected from the ORFs having the nucleotide sequence represented by SEQ ID NO: 81988.

The probes having the nucleotide sequence represented by SEQ ID NOS: 157192, 157193, 157194, 157195, 157196, 157197, 157198, 157199, 157200, 157201, 157202 were selected from the ORFs having the nucleotide sequence represented by SEQ ID NO: 81989.

The probes having the nucleotide sequence represented by SEQ ID NOS: 157203, 157204, 157205, 157206, 157207, 157208, 157209, 157210, 157211, 157212, 157213 were selected from the ORFs having the nucleotide sequence represented by SEQ ID NO: 81990.

The probes having the nucleotide sequence represented by SEQ ID NOS: 157214, 157215, 157216, 157217, 157218, 157219, 157220, 157221, 157222, 157223, 157224 were selected from the ORFs having the nucleotide sequence represented by SEQ ID NO: 81991.

The probes having the nucleotide sequence represented by SEQ ID NOS: 157225, 157226, 157227, 157228, 157229, 157230, 157231, 157232, 157233, 157234, 157235 were selected from the ORFs having the nucleotide sequence represented by SEQ ID NO: 81992.

The probes having the nucleotide sequence represented by SEQ ID NOS: 157236, 157237, 157238, 157239, 157240, 157241, 157242, 157243, 157244, 157245, 157246 were selected from the ORFs having the nucleotide sequence represented by SEQ ID NO: 81993.

The probes having the nucleotide sequence represented by SEQ ID NOS: 157247, 157248, 157249, 157250, 157251, 157252, 157253, 157254, 157255, 157256, 157257 were selected from the ORFs having the nucleotide sequence represented by SEQ ID NO: 81994.

The probes having the nucleotide sequence represented by SEQ ID NOS: 157258, 157259, 157260, 157261, 157262, 157263, 157264, 157265, 157266, 157267, 157268 were selected from the ORFs having the nucleotide sequence represented by SEQ ID NO: 81995.

The probes having the nucleotide sequence represented by SEQ ID NOS: 157269, 157270, 157271, 157272, 157273, 157274, 157275, 157276, 157277, 157278, 157279 were selected from the ORFs having the nucleotide sequence represented by SEQ ID NO: 81996.

The probes having the nucleotide sequence represented by SEQ ID NOS: 157280, 157281, 157282, 157283, 157284, 157285, 157286, 157287, 157288, 157289, 157290 were selected from the ORFs having the nucleotide sequence represented by SEQ ID NO: 81997.

The probes having the nucleotide sequence represented by SEQ ID NOS: 157291, 157292, 157293, 157294, 157295, 157296, 157297, 157298, 157299, 157300, 157301 were selected from the ORFs having the nucleotide sequence represented by SEQ ID NO: 81998.

The probes having the nucleotide sequence represented by SEQ ID NOS: 157302, 157303, 157304, 157305, 157306, 157307, 157308, 157309, 157310, 157311, 157312 were selected from the ORFs having the nucleotide sequence represented by SEQ ID NO: 81999.

The probes having the nucleotide sequence represented by SEQ ID NOS: 157313, 157314, 157315, 157316, 157317, 157318, 157319, 157320, 157321, 157322, 157323 were selected from the ORFs having the nucleotide sequence represented by SEQ ID NO: 82000.

The probes having the nucleotide sequence represented by SEQ ID NOS: 157324, 157325, 157326, 157327, 157328, 157329, 157330, 157331, 157332, 157333, 157334 were selected from the ORFs having the nucleotide sequence represented by SEQ ID NO: 82001.

The probes having the nucleotide sequence represented by SEQ ID NOS: 157335, 157336, 157337, 157338, 157339, 157340, 157341, 157342, 157343, 157344, 157345 were selected from the ORFs having the nucleotide sequence represented by SEQ ID NO: 82002.

The probes having the nucleotide sequence represented by SEQ ID NOS: 157346, 157347, 157348, 157349, 157350, 157351, 157352, 157353, 157354, 157355, 157356 were selected from the ORFs having the nucleotide sequence represented by SEQ ID NO: 82003.

The probes having the nucleotide sequence represented by SEQ ID NOS: 157357, 157358, 157359, 157360, 157361, 157362, 157363, 157364, 157365, 157366, 157367 were selected from the ORFs having the nucleotide sequence represented by SEQ ID NO: 82004.

The probes having the nucleotide sequence represented by SEQ ID NOS: 157368, 157369, 157370, 157371, 157372, 157373, 157374, 157375, 157376, 157377, 157378 were selected from the ORFs having the nucleotide sequence represented by SEQ ID NO: 82005.

The probes having the nucleotide sequence represented by SEQ ID NOS: 157379, 157380, 157381, 157382, 157383, 157384, 157385, 157386, 157387, 157388, 157389 were selected from the ORFs having the nucleotide sequence represented by SEQ ID NO: 82006.

The probes having the nucleotide sequence represented by SEQ ID NOS: 157390, 157391, 157392, 157393, 157394, 157395, 157396, 157397, 157398, 157399, 157400 were selected from the ORFs having the nucleotide sequence represented by SEQ ID NO: 82007.

The probes having the nucleotide sequence represented by SEQ ID NOS: 157401, 157402, 157403, 157404, 157405, 157406, 157407, 157408, 157409, 157410, 157411 were selected from the ORFs having the nucleotide sequence represented by SEQ ID NO: 82008.

The probes having the nucleotide sequence represented by SEQ ID NOS: 157412, 157413, 157414, 157415, 157416, 157417, 157418, 157419, 157420, 157421, 157422 were selected from the ORFs having the nucleotide sequence represented by SEQ ID NO: 82009.

The probes having the nucleotide sequence represented by SEQ ID NOS: 157423, 157424, 157425, 157426, 157427, 157428, 157429, 157430, 157431, 157432, 157433 were selected from the ORFs having the nucleotide sequence represented by SEQ ID NO: 82010.

The probes having the nucleotide sequence represented by SEQ ID NOS: 157434, 157435, 157436, 157437, 157438, 157439, 157440, 157441, 157442, 157443, 157444 were selected from the ORFs having the nucleotide sequence represented by SEQ ID NO: 82011.

The probes having the nucleotide sequence represented by SEQ ID NOS: 157445, 157446, 157447, 157448, 157449, 157450, 157451, 157452, 157453, 157454, 157455 were selected from the ORFs having the nucleotide sequence represented by SEQ ID NO: 82012.

The probes having the nucleotide sequence represented by SEQ ID NOS: 157456, 157457, 157458, 157459, 157460, 157461, 157462, 157463, 157464, 157465, 157466 were selected from the ORFs having the nucleotide sequence represented by SEQ ID NO: 82013.

The probes having the nucleotide sequence represented by SEQ ID NOS: 157467, 157468, 157469, 157470, 157471, 157472, 157473, 157474, 157475, 157476, 157477 were selected from the ORFs having the nucleotide sequence represented by SEQ ID NO: 82014.

The probes having the nucleotide sequence represented by SEQ ID NOS: 157478, 157479, 157480, 157481, 157482, 157483, 157484, 157485, 157486, 157487, 157488 were selected from the ORFs having the nucleotide sequence represented by SEQ ID NO: 82015.

The probes having the nucleotide sequence represented by SEQ ID NOS: 157489, 157490, 157491, 157492, 157493, 157494, 157495, 157496, 157497, 157498, 157499were selected from the ORFs having the nucleotide sequence represented by SEQ ID NO: 82016.

The probes having the nucleotide sequence represented by SEQ ID NOS: 157500, 157501, 157502, 157503, 157504, 157505, 157506, 157507, 157508, 157509, 157510 were selected from the ORFs having the nucleotide sequence represented by SEQ ID NO: 82017.

The probes having the nucleotide sequence represented by SEQ ID NOS: 157511, 157512, 157513, 157514, 157515, 157516, 157517, 157518, 157519, 157520, 157521 were selected from the ORFs having the nucleotide sequence represented by SEQ ID NO: 82018.

The probes having the nucleotide sequence represented by SEQ ID NOS: 157522, 157523, 157524, 157525, 157526, 157527, 157528, 157529, 157530, 157531, 157532 were selected from the ORFs having the nucleotide sequence represented by SEQ ID NO: 82019.

The probes having the nucleotide sequence represented by SEQ ID NOS: 157533, 157534, 157535, 157536, 157537, 157538, 157539, 157540, 157541, 157542, 157543 were selected from the ORFs having the nucleotide sequence represented by SEQ ID NO: 82020.

The probes having the nucleotide sequence represented by SEQ ID NOS: 157544, 157545, 157546, 157547, 157548, 157549, 157550, 157551, 157552, 157553, 157554 were selected from the ORFs having the nucleotide sequence represented by SEQ ID NO: 82021.

The probes having the nucleotide sequence represented by SEQ ID NOS: 157555, 157556, 157557, 157558, 157559, 157560 157561, 157562, 157563, 157564, 157565 were selected from the ORFs having the nucleotide sequence represented by SEQ ID NO: 82022.

The probes having the nucleotide sequence represented by SEQ ID NOS: 157566, 157567, 157568, 157569, 157570, 157571, 157572, 157573, 157574, 157575, 157576 were selected from the ORFs having the nucleotide sequence represented by SEQ ID NO: 82023.

The probes having the nucleotide sequence represented by SEQ ID NOS: 157577, 157578, 157579, 157580, 157581, 157582, 157583, 157584, 157585, 157586, 157587 were selected from the ORFs having the nucleotide sequence represented by SEQ ID NO: 82024.

The probes having the nucleotide sequence represented by SEQ ID NOS: 157588, 157589, 157590, 157591, 157592, 157593, 157594, 157595, 157596, 157597, 157598 were selected from the ORFs having the nucleotide sequence represented by SEQ ID NO: 82025.

The probes having the nucleotide sequence represented by SEQ ID NOS: 157599, 157600, 157601, 157602, 157603, 157604, 157605, 157606, 157607, 157608, 157609 were selected from the ORFs having the nucleotide sequence represented by SEQ ID NO: 82026.

The probes having the nucleotide sequence represented by SEQ ID NOS: 157610, 157611, 157612, 157613, 157614, 157615, 157616, 157617, 157618, 157619, 157620 were selected from the ORFs having the nucleotide sequence represented by SEQ ID NO: 82027.

The probes having the nucleotide sequence represented by SEQ ID NOS: 157621, 157622, 157623, 157624, 157625, 157626, 157627, 157628, 157629, 157630, 157631 were selected from the ORFs having the nucleotide sequence represented by SEQ ID NO: 82028.

The probes having the nucleotide sequence represented by SEQ ID NOS: 157632, 157633, 157634, 157635, 157636, 157637, 157638, 157639, 157640, 157641, 157642 were selected from the ORFs having the nucleotide sequence represented by SEQ ID NO: 82029.

The probes having the nucleotide sequence represented by SEQ ID NOS: 157643, 157644, 157645, 157646, 157647, 157648, 157649, 157650, 157651, 157652, 157653 were selected from the ORFs having the nucleotide sequence represented by SEQ ID NO: 82030.

The probes having the nucleotide sequence represented by SEQ ID NOS: 157654, 157655, 157656, 157657, 157658, 157659, 157660, 157661, 157662, 157663, 157664 were selected from the ORFs having the nucleotide sequence represented by SEQ ID NO: 82031.

The probes having the nucleotide sequence represented by SEQ ID NOS: 157665, 157666, 157667, 157668, 157669, 157670, 157671, 157672, 157673, 157674, 157675 were selected from the ORFs having the nucleotide sequence represented by SEQ ID NO: 82032.

The probes having the nucleotide sequence represented by SEQ ID NOS: 157676, 157677, 157678, 157679, 157680, 157681, 157682, 157683, 157684, 157685, 157686 were selected from the ORFs having the nucleotide sequence represented by SEQ ID NO: 82033.

The probes having the nucleotide sequence represented by SEQ ID NOS: 157687, 157688, 157689, 157690, 157691, 157692, 157693, 157694, 157695, 157696, 157697 were selected from the ORFs having the nucleotide sequence represented by SEQ ID NO: 82034.

The probes having the nucleotide sequence represented by SEQ ID NOS: 157698, 157699, 157700, 157701, 157702, 157703, 157704, 157705, 157706, 157707, 157708 were selected from the ORFs having the nucleotide sequence represented by SEQ ID NO: 82035.

The probes having the nucleotide sequence represented by SEQ ID NOS: 157709, 157710, 157711, 157712, 157713, 157714, 157715, 157716, 157717, 157718, 157719 were selected from the ORFs having the nucleotide sequence represented by SEQ ID NO: 82036.

The probes having the nucleotide sequence represented by SEQ ID NOS: 157720, 157721, 157722, 157723, 157724, 157725, 157726, 157727, 157728, 157729, 157730 were selected from the ORFs having the nucleotide sequence represented by SEQ ID NO: 82037.

The probes having the nucleotide sequence represented by SEQ ID NOS: 157731, 157732, 157733, 157734, 157735, 157736, 157737, 157738, 157739, 157740, 157741 were selected from the ORFs having the nucleotide sequence represented by SEQ ID NO: 82038.

The probes having the nucleotide sequence represented by SEQ ID NOS: 157742, 157743, 157744, 157745, 157746, 157747, 157748, 157749, 157750, 157751, 157752 were selected from the ORFs having the nucleotide sequence represented by SEQ ID NO: 82039.

The probes having the nucleotide sequence represented by SEQ ID NOS: 157753, 157754, 157755, 157756, 157757, 157758, 157759, 157760, 157761, 157762, 157763 were selected from the ORFs having the nucleotide sequence represented by SEQ ID NO: 82040.

The probes having the nucleotide sequence represented by SEQ ID NOS: 157764, 157765, 157766, 157767, 157768, 157769, 157770, 157771, 157772, 157773, 157774 were selected from the ORFs having the nucleotide sequence represented by SEQ ID NO: 82041.

The probes having the nucleotide sequence represented by SEQ ID NOS: 157775, 157776, 157777, 157778, 157779, 157780, 157781, 157782, 157783, 157784, 157785 were selected from the ORFs having the nucleotide sequence represented by SEQ ID NO: 82042.

The probes having the nucleotide sequence represented by SEQ ID NOS: 157786, 157787, 157788, 157789, 157790, 157791, 157792, 157793, 157794, 157795, 157796 were selected from the ORFs having the nucleotide sequence represented by SEQ ID NO: 82043.

The probes having the nucleotide sequence represented by SEQ ID NOS: 157797, 157798, 157799, 157800, 157801, 157802, 157803, 157804, 157805, 157806, 157807 were selected from the ORFs having the nucleotide sequence represented by SEQ ID NO: 82044.

The probes having the nucleotide sequence represented by SEQ ID NOS: 157808, 157809, 157810, 157811, 157812, 157813, 157814, 157815, 157816, 157817, 157818 were selected from the ORFs having the nucleotide sequence represented by SEQ ID NO: 82045.

The probes having the nucleotide sequence represented by SEQ ID NOS: 157819, 157820, 157821, 157822, 157823, 157824, 157825, 157826, 157827, 157828, 157829 were selected from the ORFs having the nucleotide sequence represented by SEQ ID NO: 82046.

The probes having the nucleotide sequence represented by SEQ ID NOS: 157830, 157831, 157832, 157833, 157834, 157835, 157836, 157837, 157838, 157839, 157840 were selected from the ORFs having the nucleotide sequence represented by SEQ ID NO: 82047.

The probes having the nucleotide sequence represented by SEQ ID NOS: 157841, 157842, 157843, 157844, 157845, 157846, 157847, 157848, 157849, 157850, 157851 were selected from the ORFs having the nucleotide sequence represented by SEQ ID NO: 82048.

The probes having the nucleotide sequence represented by SEQ ID NOS: 157852, 157853, 157854, 157855, 157856, 157857, 157858, 157859, 157860, 157861, 157862 were selected from the ORFs having the nucleotide sequence represented by SEQ ID NO: 82049.

The probes having the nucleotide sequence represented by SEQ ID NOS: 157863, 157864, 157865, 157866, 157867, 157868, 157869, 157870, 157871, 157872, 157873 were selected from the ORFs having the nucleotide sequence represented by SEQ ID NO: 82050.

The probes having the nucleotide sequence represented by SEQ ID NOS: 157874, 157875, 157876, 157877, 157878, 157879, 157880, 157881, 157882, 157883, 157884 were selected from the ORFs having the nucleotide sequence represented by SEQ ID NO: 82051.

The probes having the nucleotide sequence represented by SEQ ID NOS: 157885, 157886, 157887, 157888, 157889, 157890, 157891, 157892, 157893, 157894, 157895 were selected from the ORFs having the nucleotide sequence represented by SEQ ID NO: 82052.

The probes having the nucleotide sequence represented by SEQ ID NOS: 157896, 157897, 157898, 157899, 157900, 157901, 157902, 157903, 157904, 157905, 157906 were selected from the ORFs having the nucleotide sequence represented by SEQ ID NO: 82053.

The probes having the nucleotide sequence represented by SEQ ID NOS: 157907, 157908, 157909, 157910, 157911, 157912, 157913, 157914, 157915, 157916, 157917 were selected from the ORFs having the nucleotide sequence represented by SEQ ID NO: 82054.

The probes having the nucleotide sequence represented by SEQ ID NOS: 157918, 157919, 157920, 157921, 157922, 157923, 157924, 157925, 157926, 157927, 157928 were selected from the ORFs having the nucleotide sequence represented by SEQ ID NO: 82055.

The probes having the nucleotide sequence represented by SEQ ID NOS: 157929, 157930, 157931, 157932, 157933, 157934, 157935, 157936, 157937, 157938, 157939 were selected from the ORFs having the nucleotide sequence represented by SEQ ID NO: 82056.

The probes having the nucleotide sequence represented by SEQ ID NOS: 157940, 157941, 157942, 157943, 157944, 157945, 157946, 157947, 157948, 157949, 157950 were selected from the ORFs having the nucleotide sequence represented by SEQ ID NO: 82057.

The probes having the nucleotide sequence represented by SEQ ID NOS: 157951, 157952, 157953, 157954, 157955, 157956, 157957, 157958, 157959, 157960, 157961 were selected from the ORFs having the nucleotide sequence represented by SEQ ID NO: 82058.

The probes having the nucleotide sequence represented by SEQ ID NOS: 157962, 157963, 157964, 157965, 157966, 157967, 157968, 157969, 157970, 157971, 157972 were selected from the ORFs having the nucleotide sequence represented by SEQ ID NO: 82059.

The probes having the nucleotide sequence represented by SEQ ID NOS: 157973, 157974, 157975, 157976, 157977, 157978, 157979, 157980, 157981, 157982, 157983 were selected from the ORFs having the nucleotide sequence represented by SEQ ID NO: 82060.

The probes having the nucleotide sequence represented by SEQ ID NOS: 157984, 157985, 157986, 157987, 157988, 157989, 157990, 157991, 157992, 157993, 157994 were selected from the ORFs having the nucleotide sequence represented by SEQ ID NO: 82061.

The probes having the nucleotide sequence represented by SEQ ID NOS: 157995, 157996, 157997, 157998, 157999, 158000, 158001, 158002, 158003, 158004, 158005 were selected from the ORFs having the nucleotide sequence represented by SEQ ID NO: 82062.

The probes having the nucleotide sequence represented by SEQ ID NOS: 158006, 158007, 158008, 158009, 158010, 158011, 158012, 158013, 158014, 158015, 158016 were selected from the ORFs having the nucleotide sequence represented by SEQ ID NO: 82063.

The probes having the nucleotide sequence represented by SEQ ID NOS: 158017, 158018, 158019, 158020, 158021, 158022, 158023, 158024, 158025, 158026, 158027 were selected from the ORFs having the nucleotide sequence represented by SEQ ID NO: 82064.

The probes having the nucleotide sequence represented by SEQ ID NOS: 158028, 158029, 158030, 158031, 158032, 158033, 158034, 158035, 158036, 158037, 158038 were selected from the ORFs having the nucleotide sequence represented by SEQ ID NO: 82065.

The probes having the nucleotide sequence represented by SEQ ID NOS: 158039, 158040, 158041, 158042, 158043, 158044, 158045, 158046, 158047, 158048, 158049 were selected from the ORFs having the nucleotide sequence represented by SEQ ID NO: 82066.

The probes having the nucleotide sequence represented by SEQ ID NOS: 158050, 158051, 158052, 158053, 158054, 158055, 158056, 158057, 158058, 158059, 158060 were selected from the ORFs having the nucleotide sequence represented by SEQ ID NO: 82067.

The probes having the nucleotide sequence represented by SEQ ID NOS: 158061, 158062, 158063, 158064, 158065, 158066, 158067, 158068, 158069, 158070, 158071 were selected from the ORFs having the nucleotide sequence represented by SEQ ID NO: 82068.

The probes having the nucleotide sequence represented by SEQ ID NOS: 158072, 158073, 158074, 158075, 158076, 158077, 158078, 158079, 158080, 158081, 158082 were selected from the ORFs having the nucleotide sequence represented by SEQ ID NO: 82069.

The probes having the nucleotide sequence represented by SEQ ID NOS: 158083, 158084, 158085, 158086, 158087, 158088, 158089, 158090, 158091, 158092, 158093were selected from the ORFs having the nucleotide sequence represented by SEQ ID NO: 82070.

The probes having the nucleotide sequence represented by SEQ ID NOS: 158094, 158095, 158096, 158097, 158098, 158099, 158100, 158101, 158102, 158103, 158104 were selected from the ORFs having the nucleotide sequence represented by SEQ ID NO: 82071.

The probes having the nucleotide sequence represented by SEQ ID NOS: 158105, 158106, 158107, 158108, 158109, 158110, 158111, 158112, 158113, 158114, 158115 were selected from the ORFs having the nucleotide sequence represented by SEQ ID NO: 82072.

The probes having the nucleotide sequence represented by SEQ ID NOS: 158116, 158117, 158118, 158119, 158120, 158121, 158122, 158123, 158124, 158125, 158126 were selected from the ORFs having the nucleotide sequence represented by SEQ ID NO: 82073.

The probes having the nucleotide sequence represented by SEQ ID NOS: 158127, 158128, 158129, 158130, 158131, 158132, 158133, 158134, 158135, 158136, 158137, 158138, 158139, 158140, 158141, 158142, 158143, 158144, 158145, 158146, 158147 were selected from the ORFs having the nucleotide sequence represented by SEQ ID NO: 82074.

The probes having the nucleotide sequence represented by SEQ ID NOS: 158148, 158149, 158150, 158151, 158152, 158153, 158154, 158155, 158156, 158157, 158158 were selected from the ORFs having the nucleotide sequence represented by SEQ ID NO: 82075.

The probes having the nucleotide sequence represented by SEQ ID NOS: 158159, 158160, 158161, 158162, 158163, 158164, 158165, 158166, 158167, 158168, 158169 were selected from the ORFs having the nucleotide sequence represented by SEQ ID NO: 82076.

The probes having the nucleotide sequence represented by SEQ ID NOS: 158170, 158171, 158172, 158173, 158174, 158175, 158176, 158177, 158178, 158179, 158180 were selected from the ORFs having the nucleotide sequence represented by SEQ ID NO: 82077.

The probes having the nucleotide sequence represented by SEQ ID NOS: 158181, 158182, 158183, 158184, 158185, 158186, 158187, 158188, 158189, 158190, 158191 were selected from the ORFs having the nucleotide sequence represented by SEQ ID NO: 82078.

The probes having the nucleotide sequence represented by SEQ ID NOS: 158192, 158193, 158194, 158195, 158196, 158197, 158198, 158199, 158200, 158201, 158202 were selected from the ORFs having the nucleotide sequence represented by SEQ ID NO: 82079.

The probes having the nucleotide sequence represented by SEQ ID NOS: 158203, 158204, 158205, 158206, 158207, 158208, 158209, 158210, 158211, 158212, 158213 were selected from the ORFs having the nucleotide sequence represented by SEQ ID NO: 82080.

The probes having the nucleotide sequence represented by SEQ ID NOS: 158214, 158215, 158216, 158217, 158218, 158219, 158220, 158221, 158222, 158223, 158224 were selected from the ORFs having the nucleotide sequence represented by SEQ ID NO: 82081.

The probes having the nucleotide sequence represented by SEQ ID NOS: 158225, 158226, 158227, 158228, 158229, 158230, 158231, 158232, 158233, 158234, 158235 were selected from the ORFs having the nucleotide sequence represented by SEQ ID NO: 82082.

The probes having the nucleotide sequence represented by SEQ ID NOS: 158236, 158237, 158238, 158239, 158240, 158241, 158242, 158243, 158244, 158245, 158246 were selected from the ORFs having the nucleotide sequence represented by SEQ ID NO: 82083.

The probes having the nucleotide sequence represented by SEQ ID NOS: 158247, 158248, 158249, 158250, 158251, 158252, 158253, 158254, 158255, 158256, 158257 were selected from the ORFs having the nucleotide sequence represented by SEQ ID NO: 82084.

The probes having the nucleotide sequence represented by SEQ ID NOS: 158258, 158259, 158260, 158261, 158262, 158263, 158264, 158265, 158266, 158267, 158268 were selected from the ORFs having the nucleotide sequence represented by SEQ ID NO: 82085.

The probes having the nucleotide sequence represented by SEQ ID NOS: 158269, 158270, 158271, 158272, 158273, 158274, 158275, 158276, 158277, 158278, 158279 were selected from the ORFs having the nucleotide sequence represented by SEQ ID NO: 82086.

The probes having the nucleotide sequence represented by SEQ ID NOS: 158280, 158281, 158282, 158283, 158284, 158285, 158286, 158287, 158288, 158289, 158290 were selected from the ORFs having the nucleotide sequence represented by SEQ ID NO: 82087.

The probes having the nucleotide sequence represented by SEQ ID NOS: 158291, 158292, 158293, 158294, 158295, 158296, 158297, 158298, 158299, 158300, 158301 were selected from the ORFs having the nucleotide sequence represented by SEQ ID NO: 82088.

The probes having the nucleotide sequence represented by SEQ ID NOS: 158302, 158303, 158304, 158305, 158306, 158307, 158308, 158309, 158310, 158311, 158312 were selected from the ORFs having the nucleotide sequence represented by SEQ ID NO: 82089.

The probes having the nucleotide sequence represented by SEQ ID NOS: 158313, 158314, 158315, 158316, 158317, 158318, 158319, 158320, 158321, 158322, 158323 were selected from the ORFs having the nucleotide sequence represented by SEQ ID NO: 82090.

The probes having the nucleotide sequence represented by SEQ ID NOS: 158324, 158325, 158326, 158327, 158328, 158329, 158330, 158331, 158332, 158333, 158334, 158335, 158336, 158337, 158338, 158339, 158340, 158341, 158342, 158343, 158344, 158345 were selected from the ORFs having the nucleotide sequence represented by SEQ ID NO: 82091.

The probes having the nucleotide sequence represented by SEQ ID NOS: 158346, 158347, 158348, 158349, 158350, 158351, 158352, 158353, 158354, 158355, 158356 were selected from the ORFs having the nucleotide sequence represented by SEQ ID NO: 82092.

The probes having the nucleotide sequence represented by SEQ ID NOS: 158357, 158358, 158359, 158360, 158361, 158362, 158363, 158364, 158365, 158366, 158367 were selected from the ORFs having the nucleotide sequence represented by SEQ ID NO: 82093.

The probes having the nucleotide sequence represented by SEQ ID NOS: 158368, 158369, 158370, 158371, 158372, 158373, 158374, 158375, 158376, 158377, 158378 were selected from the ORFs having the nucleotide sequence represented by SEQ ID NO: 82094.

The probes having the nucleotide sequence represented by SEQ ID NOS: 158379, 158380, 158381, 158382, 158383, 158384, 158385, 158386, 158387, 158388, 158389 were selected from the ORFs having the nucleotide sequence represented by SEQ ID NO: 82095.

The probes having the nucleotide sequence represented by SEQ ID NOS: 158390, 158391, 158392, 158393, 158394, 158395, 158396, 158397, 158398, 158399, 158400, 158401, 158402, 158403, 158404, 158405, 158406, 158407, 158408, 158409, 158410, 158411 were selected from the ORFs having the nucleotide sequence represented by SEQ ID NO: 82096.

The probes having the nucleotide sequence represented by SEQ ID NOS: 158412, 158413, 158414, 158415, 158416, 158417, 158418, 158419, 158420, 158421, 158422 were selected from the ORFs having the nucleotide sequence represented by SEQ ID NO: 82097.

The probes having the nucleotide sequence represented by SEQ ID NOS: 158423, 158424, 158425, 158426, 158427, 158428, 158429, 158430, 158431, 158432, 158433 were selected from the ORFs having the nucleotide sequence represented by SEQ ID NO: 82098.

The probes having the nucleotide sequence represented by SEQ ID NOS: 158434, 158435, 158436, 158437, 158438, 158439, 158440, 158441, 158442, 158443, 158444 were selected from the ORFs having the nucleotide sequence represented by SEQ ID NO: 82099.

The probes having the nucleotide sequence represented by SEQ ID NOS: 158445, 158446, 158447, 158448, 158449, 158450, 158451, 158452, 158453, 158454, 158455 were selected from the ORFs having the nucleotide sequence represented by SEQ ID NO: 82100.

The probes having the nucleotide sequence represented by SEQ ID NOS: 158456, 158457, 158458, 158459, 158460, 158461, 158462, 158463, 158464, 158465, 158466 were selected from the ORFs having the nucleotide sequence represented by SEQ ID NO: 82101.

The probes having the nucleotide sequence represented by SEQ ID NOS: 158467, 158468, 158469, 158470, 158471, 158472, 158473, 158474, 158475, 158476, 158477 were selected from the ORFs having the nucleotide sequence represented by SEQ ID NO: 82102.

The probes having the nucleotide sequence represented by SEQ ID NOS: 158478, 158479, 158480, 158481, 158482, 158483, 158484, 158485, 158486, 158487, 158488 were selected from the ORFs having the nucleotide sequence represented by SEQ ID NO: 82103.

The probes having the nucleotide sequence represented by SEQ ID NOS: 158489, 158490, 158491, 158492, 158493, 158494, 158495, 158496, 158497, 158498, 158499 were selected from the ORFs having the nucleotide sequence represented by SEQ ID NO: 82104.

The probes having the nucleotide sequence represented by SEQ ID NOS: 158500, 158501, 158502, 158503, 158504, 158505, 158506, 158507, 158508, 158509, 158510 were selected from the ORFs having the nucleotide sequence represented by SEQ ID NO: 82105.

The probes having the nucleotide sequence represented by SEQ ID NOS: 158511, 158512, 158513, 158514, 158515, 158516, 158517, 158518, 158519, 158520, 158521 were selected from the ORFs having the nucleotide sequence represented by SEQ ID NO: 82106.

The probes having the nucleotide sequence represented by SEQ ID NOS: 158522, 158523, 158524, 158525, 158526, 158527, 158528, 158529, 158530, 158531, 158532 were selected from the ORFs having the nucleotide sequence represented by SEQ ID NO: 82107.

The probes having the nucleotide sequence represented by SEQ ID NOS: 158533, 158534, 158535, 158536, 158537, 158538, 158539, 158540, 158541, 158542, 158543 were selected from the ORFs having the nucleotide sequence represented by SEQ ID NO: 82108.

The probes having the nucleotide sequence represented by SEQ ID NOS: 158544, 158545, 158546, 158547, 158548, 158549, 158550, 158551, 158552, 158553, 158554 were selected from the ORFs having the nucleotide sequence represented by SEQ ID NO: 82109.

The probes having the nucleotide sequence represented by SEQ ID NOS: 158555, 158556, 158557, 158558, 158559, 158560, 158561, 158562, 158563, 158564, 158565 were selected from the ORFs having the nucleotide sequence represented by SEQ ID NO: 82110.

The probes having the nucleotide sequence represented by SEQ ID NOS: 158566, 158567, 158568, 158569, 158570, 158571, 158572, 158573, 158574, 158575, 158576 were selected from the ORFs having the nucleotide sequence represented by SEQ ID NO: 82111.

The probes having the nucleotide sequence represented by SEQ ID NOS: 158577, 158578, 158579, 158580, 158581, 158582, 158583, 158584, 158585, 158586, 158587 were selected from the ORFs having the nucleotide sequence represented by SEQ ID NO: 82112.

The probes having the nucleotide sequence represented by SEQ ID NOS: 158588, 158589, 158590, 158591, 158592, 158593, 158594, 158595, 158596, 158597, 158598 were selected from the ORFs having the nucleotide sequence represented by SEQ ID NO: 82113.

The probes having the nucleotide sequence represented by SEQ ID NOS: 158599, 158600, 158601, 158602, 158603, 158604, 158605, 158606, 158607, 158608, 158609 were selected from the ORFs having the nucleotide sequence represented by SEQ ID NO: 82114.

The probes having the nucleotide sequence represented by SEQ ID NOS: 158610, 158611, 158612, 158613, 158614, 158615, 158616, 158617, 158618, 158619, 158620 were selected from the ORFs having the nucleotide sequence represented by SEQ ID NO: 82115.

The probes having the nucleotide sequence represented by SEQ ID NOS: 158621, 158622, 158623, 158624, 158625, 158626, 158627, 158628, 158629, 158630, 158631 were selected from the ORFs having the nucleotide sequence represented by SEQ ID NO: 82116.

The probes having the nucleotide sequence represented by SEQ ID NOS: 158632, 158633, 158634, 158635, 158636, 158637, 158638, 158639, 158640, 158641, 158642 were selected from the ORFs having the nucleotide sequence represented by SEQ ID NO: 82117.

The probes having the nucleotide sequence represented by SEQ ID NOS: 158643, 158644, 158645, 158646, 158647, 158648, 158649, 158650, 158651, 158652, 158653 were selected from the ORFs having the nucleotide sequence represented by SEQ ID NO: 82118.

The probes having the nucleotide sequence represented by SEQ ID NOS: 158654, 158655, 158656, 158657, 158658, 158659, 158660, 158661, 158662, 158663, 158664 were selected from the ORFs having the nucleotide sequence represented by SEQ ID NO: 82119.

The probes having the nucleotide sequence represented by SEQ ID NOS: 158665, 158666, 158667, 158668, 158669, 158670, 158671, 158672, 158673, 158674, 158675 were selected from the ORFs having the nucleotide sequence represented by SEQ ID NO: 82120.

The probes having the nucleotide sequence represented by SEQ ID NOS: 158676, 158677, 158678, 158679, 158680, 158681, 158682, 158683, 158684, 158685, 158686 were selected from the ORFs having the nucleotide sequence represented by SEQ ID NO: 82121.

The probes having the nucleotide sequence represented by SEQ ID NOS: 158687, 158688, 158689, 158690, 158691, 158692, 158693, 158694, 158695, 158696, 158697 were selected from the ORFs having the nucleotide sequence represented by SEQ ID NO: 82122.

The probes having the nucleotide sequence represented by SEQ ID NOS: 158698, 158699, 158700, 158701, 158702, 158703, 158704, 158705, 158706, 158707, 158708 were selected from the ORFs having the nucleotide sequence represented by SEQ ID NO: 82123.

The probes having the nucleotide sequence represented by SEQ ID NOS: 158709, 158710, 158711, 158712, 158713, 158714, 158715, 158716, 158717, 158718, 158719 were selected from the ORFs having the nucleotide sequence represented by SEQ ID NO: 82124.

The probes having the nucleotide sequence represented by SEQ ID NOS: 158720, 158721, 158722, 158723, 158724, 158725, 158726, 158727, 158728, 158729, 158730 were selected from the ORFs having the nucleotide sequence represented by SEQ ID NO: 82125.

The probes having the nucleotide sequence represented by SEQ ID NOS: 158731, 158732, 158733, 158734, 158735, 158736, 158737, 158738, 158739, 158740, 158741 were selected from the ORFs having the nucleotide sequence represented by SEQ ID NO: 82126.

The probes having the nucleotide sequence represented by SEQ ID NOS: 158742, 158743, 158744, 158745, 158746, 158747, 158748, 158749, 158750, 158751, 158752 were selected from the ORFs having the nucleotide sequence represented by SEQ ID NO: 82127.

The probes having the nucleotide sequence represented by SEQ ID NOS: 158753, 158754, 158755, 158756, 158757, 158758, 158759, 158760, 158761, 158762, 158763 were selected from the ORFs having the nucleotide sequence represented by SEQ ID NO: 82128.

The probes having the nucleotide sequence represented by SEQ ID NOS: 158764, 158765, 158766, 158767, 158768, 158769, 158770, 158771, 158772, 158773, 158774 were selected from the ORFs having the nucleotide sequence represented by SEQ ID NO: 82129.

The probes having the nucleotide sequence represented by SEQ ID NOS: 158775, 158776, 158777, 158778, 158779, 158780, 158781, 158782, 158783, 158784, 158785 were selected from the ORFs having the nucleotide sequence represented by SEQ ID NO: 82130.

The probes having the nucleotide sequence represented by SEQ ID NOS: 158786, 158787, 158788, 158789, 158790, 158791, 158792, 158793, 158794, 158795, 158796 were selected from the ORFs having the nucleotide sequence represented by SEQ ID NO: 82131.

The probes having the nucleotide sequence represented by SEQ ID NOS: 158797, 158798, 158799, 158800, 158801, 158802, 158803, 158804, 158805, 158806, 158807 were selected from the ORFs having the nucleotide sequence represented by SEQ ID NO: 82132.

The probes having the nucleotide sequence represented by SEQ ID NOS: 158808, 158809, 158810, 158811, 158812, 158813, 158814, 158815, 158816, 158817, 158818 were selected from the ORFs having the nucleotide sequence represented by SEQ ID NO: 82133.

The probes having the nucleotide sequence represented by SEQ ID NOS: 158819, 158820, 158821, 158822, 158823, 158824, 158825, 158826, 158827, 158828, 158829 were selected from the ORFs having the nucleotide sequence represented by SEQ ID NO: 82134.

The probes having the nucleotide sequence represented by SEQ ID NOS: 158830, 158831, 158832, 158833, 158834, 158835, 158836, 158837, 158838, 158839, 158840 were selected from the ORFs having the nucleotide sequence represented by SEQ ID NO: 82135.

The probes having the nucleotide sequence represented by SEQ ID NOS: 158841, 158842, 158843, 158844, 158845, 158846, 158847, 158848, 158849, 158850, 158851 were selected from the ORFs having the nucleotide sequence represented by SEQ ID NO: 82136.

The probes having the nucleotide sequence represented by SEQ ID NOS: 158852, 158853, 158854, 158855, 158856, 158857, 158858, 158859, 158860, 158861, 158862 were selected from the ORFs having the nucleotide sequence represented by SEQ ID NO: 82137.

The probes having the nucleotide sequence represented by SEQ ID NOS: 158863, 158864, 158865, 158866, 158867, 158868, 158869, 158870, 158871, 158872, 158873 were selected from the ORFs having the nucleotide sequence represented by SEQ ID NO: 82138.

The probes having the nucleotide sequence represented by SEQ ID NOS: 158874, 158875, 158876, 158877, 158878, 158879, 158880, 158881, 158882, 158883, 158884 were selected from the ORFs having the nucleotide sequence represented by SEQ ID NO: 82139.

The probes having the nucleotide sequence represented by SEQ ID NOS: 158885, 158886, 158887, 158888, 158889, 158890, 158891, 158892, 158893, 158894, 158895 were selected from the ORFs having the nucleotide sequence represented by SEQ ID NO: 82140.

The probes having the nucleotide sequence represented by SEQ ID NOS: 158896, 158897, 158898, 158899, 158900, 158901, 158902, 158903, 158904, 158905, 158906 were selected from the ORFs having the nucleotide sequence represented by SEQ ID NO: 82141.

The probes having the nucleotide sequence represented by SEQ ID NOS: 158907, 158908, 158909, 158910, 158911, 158912, 158913, 158914, 158915, 158916, 158917 were selected from the ORFs having the nucleotide sequence represented by SEQ ID NO: 82142.

The probes having the nucleotide sequence represented by SEQ ID NOS: 158918, 158919, 158920, 158921, 158922, 158923, 158924, 158925, 158926, 158927, 158928 were selected from the ORFs having the nucleotide sequence represented by SEQ ID NO: 82143.

The probes having the nucleotide sequence represented by SEQ ID NOS: 158929, 158930, 158931, 158932, 158933, 158934, 158935, 158936, 158937, 158938, 158939 were selected from the ORFs having the nucleotide sequence represented by SEQ ID NO: 82144.

The probes having the nucleotide sequence represented by SEQ ID NOS: 158940, 158941, 158942, 158943, 158944, 158945, 158946, 158947, 158948, 158949, 158950 were selected from the ORFs having the nucleotide sequence represented by SEQ ID NO: 82145.

The probes having the nucleotide sequence represented by SEQ ID NOS: 158951, 158952, 158953, 158954, 158955, 158956, 158957, 158958, 158959, 158960, 158961 were selected from the ORFs having the nucleotide sequence represented by SEQ ID NO: 82146.

The probes having the nucleotide sequence represented by SEQ ID NOS: 158962, 158963, 158964, 158965, 158966, 158967, 158968, 158969, 158970, 158971, 158972 were selected from the ORFs having the nucleotide sequence represented by SEQ ID NO: 82147.

The probes having the nucleotide sequence represented by SEQ ID NOS: 158973, 158974, 158975, 158976, 158977, 158978, 158979, 158980, 158981, 158982, 158983 were selected from the ORFs having the nucleotide sequence represented by SEQ ID NO: 82148.

The probes having the nucleotide sequence represented by SEQ ID NOS: 158984, 158985, 158986, 158987, 158988, 158989, 158990, 158991, 158992, 158993, 158994 were selected from the ORFs having the nucleotide sequence represented by SEQ ID NO: 82149.

The probes having the nucleotide sequence represented by SEQ ID NOS: 158995, 158996, 158997, 158998, 158999, 159000, 159001, 159002, 159003, 159004, 159005 were selected from the ORFs having the nucleotide sequence represented by SEQ ID NO: 82150.

The probes having the nucleotide sequence represented by SEQ ID NOS: 159006, 159007, 159008, 159009, 159010, 159011, 159012, 159013, 159014, 159015, 159016 were selected from the ORFs having the nucleotide sequence represented by SEQ ID NO: 82151.

The probes having the nucleotide sequence represented by SEQ ID NOS: 159017, 159018, 159019, 159020, 159021, 159022, 159023, 159024, 159025, 159026, 159027 were selected from the ORFs having the nucleotide sequence represented by SEQ ID NO: 82152.

The probes having the nucleotide sequence represented by SEQ ID NOS: 159028, 159029, 159030, 159031, 159032, 159033, 159034, 159035, 159036, 159037, 159038 were selected from the ORFs having the nucleotide sequence represented by SEQ ID NO: 82153.

The probes having the nucleotide sequence represented by SEQ ID NOS: 159039, 159040, 159041, 159042, 159043, 159044, 159045, 159046, 159047, 159048, 159049 were selected from the ORFs having the nucleotide sequence represented by SEQ ID NO: 82154.

The probes having the nucleotide sequence represented by SEQ ID NOS: 159050, 159051, 159052, 159053, 159054, 159055, 159056, 159057, 159058, 159059, 159060 were selected from the ORFs having the nucleotide sequence represented by SEQ ID NO: 82155.

The probes having the nucleotide sequence represented by SEQ ID NOS: 159061, 159062, 159063, 159064, 159065, 159066, 159067, 159068, 159069, 159070, 159071 were selected from the ORFs having the nucleotide sequence represented by SEQ ID NO: 82156.

The probes having the nucleotide sequence represented by SEQ ID NOS: 159072, 159073, 159074, 159075, 159076, 159077, 159078, 159079, 159080, 159081, 159082 were selected from the ORFs having the nucleotide sequence represented by SEQ ID NO: 82157.

The probes having the nucleotide sequence represented by SEQ ID NOS: 159083, 159084, 159085, 159086, 159087, 159088, 159089, 159090, 159091, 159092, 159093 were selected from the ORFs having the nucleotide sequence represented by SEQ ID NO: 82158.

The probes having the nucleotide sequence represented by SEQ ID NOS: 159094, 159095, 159096, 159097, 159098, 159099, 159100, 159101, 159102, 159103, 159104 were selected from the ORFs having the nucleotide sequence represented by SEQ ID NO: 82159.

The probes having the nucleotide sequence represented by SEQ ID NOS: 159105, 159106, 159107, 159108, 159109, 159110, 159111, 159112, 159113, 159114, 159115 were selected from the ORFs having the nucleotide sequence represented by SEQ ID NO: 82160.

The probes having the nucleotide sequence represented by SEQ ID NOS: 159116, 159117, 159118, 159119, 159120, 159121, 159122, 159123, 159124, 159125, 159126 were selected from the ORFs having the nucleotide sequence represented by SEQ ID NO: 82161.

The probes having the nucleotide sequence represented by SEQ ID NOS: 159127, 159128, 159129, 159130, 159131, 159132, 159133, 159134, 159135, 159136, 159137 were selected from the ORFs having the nucleotide sequence represented by SEQ ID NO: 82162.

The probes having the nucleotide sequence represented by SEQ ID NOS: 159138, 159139, 159140, 159141, 159142, 159143, 159144, 159145, 159146, 159147, 159148 were selected from the ORFs having the nucleotide sequence represented by SEQ ID NO: 82163.

The probes having the nucleotide sequence represented by SEQ ID NOS: 159149, 159150, 159151, 159152, 159153, 159154, 159155, 159156, 159157, 159158, 159159 were selected from the ORFs having the nucleotide sequence represented by SEQ ID NO: 82164.

The probes having the nucleotide sequence represented by SEQ ID NOS: 159160, 159161, 159162, 159163, 159164, 159165, 159166, 159167, 159168, 159169, 159170 were selected from the ORFs having the nucleotide sequence represented by SEQ ID NO: 82165.

The probes having the nucleotide sequence represented by SEQ ID NOS: 159171, 159172, 159173, 159174, 159175, 159176, 159177, 159178, 159179, 159180, 159181 were selected from the ORFs having the nucleotide sequence represented by SEQ ID NO: 82166.

The probes having the nucleotide sequence represented by SEQ ID NOS: 159182, 159183, 159184, 159185, 159186, 159187, 159188, 159189, 159190, 159191, 159192 were selected from the ORFs having the nucleotide sequence represented by SEQ ID NO: 82167.

The probes having the nucleotide sequence represented by SEQ ID NOS: 159193, 159194, 159195, 159196, 159197, 159198, 159199, 159200, 159201, 159202, 159203 were selected from the ORFs having the nucleotide sequence represented by SEQ ID NO: 82168.

The probes having the nucleotide sequence represented by SEQ ID NOS: 159204, 159205, 159206, 159207, 159208, 159209, 159210, 159211, 159212, 159213, 159214 were selected from the ORFs having the nucleotide sequence represented by SEQ ID NO: 82169.

The probes having the nucleotide sequence represented by SEQ ID NOS: 159215, 159216, 159217, 159218, 159219, 159220, 159221, 159222, 159223, 159224, 159225, 159226, 159227, 159228, 159229, 159230, 159231, 159232, 159233 were selected from the ORFs having the nucleotide sequence represented by SEQ ID NO: 82170.

The probes having the nucleotide sequence represented by SEQ ID NOS: 159234, 159235, 159236, 159237, 159238, 159239, 159240, 159241, 159242, 159243, 159244 were selected from the ORFs having the nucleotide sequence represented by SEQ ID NO: 82171.

The probes having the nucleotide sequence represented by SEQ ID NOS: 159245, 159246, 159247, 159248, 159249, 159250, 159251, 159252, 159253, 159254, 159255 were selected from the ORFs having the nucleotide sequence represented by SEQ ID NO: 82172.

The probes having the nucleotide sequence represented by SEQ ID NOS: 159256, 159257, 159258, 159259, 159260, 159261, 159262, 159263, 159264, 159265, 159266 were selected from the ORFs having the nucleotide sequence represented by SEQ ID NO: 82173.

The probes having the nucleotide sequence represented by SEQ ID NOS: 159267, 159268, 159269, 159270, 159271, 159272, 159273, 159274, 159275, 159276, 159277 were selected from the ORFs having the nucleotide sequence represented by SEQ ID NO: 82174.

The probes having the nucleotide sequence represented by SEQ ID NOS: 159278, 159279, 159280, 159281, 159282, 159283, 159284, 159285, 159286, 159287, 159288 were selected from the ORFs having the nucleotide sequence represented by SEQ ID NO: 82175.

The probes having the nucleotide sequence represented by SEQ ID NOS: 159289, 159290, 159291, 159292, 159293, 159294, 159295, 159296, 159297, 159298, 159299 were selected from the ORFs having the nucleotide sequence represented by SEQ ID NO: 82176.

The probes having the nucleotide sequence represented by SEQ ID NOS: 159300, 159301, 159302, 159303, 159304, 159305, 159306, 159307, 159308, 159309, 159310 were selected from the ORFs having the nucleotide sequence represented by SEQ ID NO: 82177.

The probes having the nucleotide sequence represented by SEQ ID NOS: 159311, 159312, 159313, 159314, 159315, 159316, 159317, 159318, 159319, 159320, 159321, 159322, 159323, 159324, 159325, 159326, 159327, 159328, 159329, 159330, 159331, 159332 were selected from the ORFs having the nucleotide sequence represented by SEQ ID NO: 82178.

The probes having the nucleotide sequence represented by SEQ ID NOS: 159333, 159334, 159335, 159336, 159337, 159338, 159339, 159340, 159341, 159342, 159343 were selected from the ORFs having the nucleotide sequence represented by SEQ ID NO: 82179.

The probes having the nucleotide sequence represented by SEQ ID NOS: 159344, 159345, 159346, 159347, 159348, 159349, 159350, 159351, 159352, 159353, 159354 were selected from the ORFs having the nucleotide sequence represented by SEQ ID NO: 82180.

The probes having the nucleotide sequence represented by SEQ ID NOS: 159355, 159356, 159357, 159358, 159359, 159360, 159361, 159362, 159363, 159364, 159365 were selected from the ORFs having the nucleotide sequence represented by SEQ ID NO: 82181.

The probes having the nucleotide sequence represented by SEQ ID NOS: 159366, 159367, 159368, 159369, 159370, 159371, 159372, 159373, 159374, 159375, 159376 were selected from the ORFs having the nucleotide sequence represented by SEQ ID NO: 82182.

The probes having the nucleotide sequence represented by SEQ ID NOS: 159377, 159378, 159379, 159380, 159381, 159382, 159383, 159384, 159385, 159386, 159387 were selected from the ORFs having the nucleotide sequence represented by SEQ ID NO: 82183.

The probes having the nucleotide sequence represented by SEQ ID NOS: 159388, 159389, 159390, 159391, 159392, 159393, 159394, 159395, 159396, 159397, 159398 were selected from the ORFs having the nucleotide sequence represented by SEQ ID NO: 82184.

The probes having the nucleotide sequence represented by SEQ ID NOS: 159399, 159400, 159401, 159402, 159403, 159404, 159405, 159406, 159407, 159408, 159409 were selected from the ORFs having the nucleotide sequence represented by SEQ ID NO: 82185.

The probes having the nucleotide sequence represented by SEQ ID NOS: 159410, 159411, 159412, 159413, 159414, 159415, 159416, 159417, 159418, 159419, 159420 were selected from the ORFs having the nucleotide sequence represented by SEQ ID NO: 82186.

The probes having the nucleotide sequence represented by SEQ ID NOS: 159421, 159422, 159423, 159424, 159425, 159426, 159427, 159428, 159429, 159430, 159431 were selected from the ORFs having the nucleotide sequence represented by SEQ ID NO: 82187.

The probes having the nucleotide sequence represented by SEQ ID NOS: 159432, 159433, 159434, 159435, 159436, 159437, 159438, 159439, 159440, 159441, 159442 were selected from the ORFs having the nucleotide sequence represented by SEQ ID NO: 82188.

The probes having the nucleotide sequence represented by SEQ ID NOS: 159443, 159444, 159445, 159446, 159447, 159448, 159449, 159450, 159451, 159452, 159453, 159454, 159455, 159456, 159457, 159458, 159459, 159460, 159461, 159462, 159463, 159464 were selected from the ORFs having the nucleotide sequence represented by SEQ ID NO: 82189.

The probes having the nucleotide sequence represented by SEQ ID NOS: 159465, 159466, 159467, 159468, 159469, 159470, 159471, 159472, 159473 were selected from the ORFs having the nucleotide sequence represented by SEQ ID NO: 82190.

The probes having the nucleotide sequence represented by SEQ ID NOS: 159474, 159475, 159476, 159477, 159478, 159479, 159480, 159481, 159482, 159483, 159484 were selected from the ORFs having the nucleotide sequence represented by SEQ ID NO: 82191.

The probes having the nucleotide sequence represented by SEQ ID NOS: 159485, 159486, 159487, 159488, 159489, 159490, 159491, 159492, 159493, 159494, 159495 were selected from the ORFs having the nucleotide sequence represented by SEQ ID NO: 82192.

The probes having the nucleotide sequence represented by SEQ ID NOS: 159496, 159497, 159498, 159499, 159500, 159501, 159502, 159503, 159504, 159505, 159506, 159507, 159508, 159509, 159510, 159511, 159512, 159513, 159514, 159515, 159516, 159517 were selected from the ORFs having the nucleotide sequence represented by SEQ ID NO: 82193.

The probes having the nucleotide sequence represented by SEQ ID NOS: 159518, 159519, 159520, 159521, 159522, 159523, 159524, 159525, 159526, 159527, 159528 were selected from the ORFs having the nucleotide sequence represented by SEQ ID NO: 82194.

The probes having the nucleotide sequence represented by SEQ ID NOS: 159529, 159530, 159531, 159532, 159533, 159534, 159535, 159536, 159537, 159538, 159539 were selected from the ORFs having the nucleotide sequence represented by SEQ ID NO: 82195.

The probes having the nucleotide sequence represented by SEQ ID NOS: 159540, 159541, 159542, 159543, 159544, 159545, 159546, 159547, 159548, 159549, 159550 were selected from the ORFs having the nucleotide sequence represented by SEQ ID NO: 82196.

The probes having the nucleotide sequence represented by SEQ ID NOS: 159551, 159552, 159553, 159554, 159555, 159556, 159557, 159558, 159559, 159560, 159561 were selected from the ORFs having the nucleotide sequence represented by SEQ ID NO: 82197.

The probes having the nucleotide sequence represented by SEQ ID NOS: 159562, 159563, 159564, 159565, 159566, 159567, 159568, 159569, 159570, 159571, 159572 were selected from the ORFs having the nucleotide sequence represented by SEQ ID NO: 82198.

The probes having the nucleotide sequence represented by SEQ ID NOS: 159573, 159574, 159575, 159576, 159577, 159578, 159579, 159580, 159581, 159582, 159583 were selected from the ORFs having the nucleotide sequence represented by SEQ ID NO: 82199.

The probes having the nucleotide sequence represented by SEQ ID NOS: 159584, 159585, 159586, 159587, 159588, 159589, 159590, 159591, 159592, 159593, 159594 were selected from the ORFs having the nucleotide sequence represented by SEQ ID NO: 82200.

The probes having the nucleotide sequence represented by SEQ ID NOS: 159595, 159596, 159597, 159598, 159599, 159600, 159601, 159602, 159603, 159604, 159605 were selected from the ORFs having the nucleotide sequence represented by SEQ ID NO: 82201.

The probes having the nucleotide sequence represented by SEQ ID NOS: 159606, 159607, 159608, 159609, 159610, 159611, 159612, 159613, 159614, 159615, 159616 were selected from the ORFs having the nucleotide sequence represented by SEQ ID NO: 82202.

The probes having the nucleotide sequence represented by SEQ ID NOS: 159617, 159618, 159619, 159620, 159621, 159622, 159623, 159624, 159625, 159626, 159627 were selected from the ORFs having the nucleotide sequence represented by SEQ ID NO: 82203.

The probes having the nucleotide sequence represented by SEQ ID NOS: 159628, 159629, 159630, 159631, 159632, 159633, 159634, 159635, 159636, 159637, 159638 were selected from the ORFs having the nucleotide sequence represented by SEQ ID NO: 82204.

The probes having the nucleotide sequence represented by SEQ ID NOS: 159639, 159640, 159641, 159642, 159643, 159644, 159645, 159646, 159647, 159648, 159649 were selected from the ORFs having the nucleotide sequence represented by SEQ ID NO: 82205.

The probes having the nucleotide sequence represented by SEQ ID NOS: 159650, 159651, 159652, 159653, 159654, 159655, 159656, 159657, 159658, 159659, 159660 were selected from the ORFs having the nucleotide sequence represented by SEQ ID NO: 82206.

The probes having the nucleotide sequence represented by SEQ ID NOS: 159661, 159662, 159663, 159664, 159665, 159666, 159667, 159668, 159669, 159670, 159671 were selected from the ORFs having the nucleotide sequence represented by SEQ ID NO: 82207.

The probes having the nucleotide sequence represented by SEQ ID NOS: 159672, 159673, 159674, 159675, 159676, 159677, 159678, 159679, 159680, 159681, 159682 were selected from the ORFs having the nucleotide sequence represented by SEQ ID NO: 82208.

The probes having the nucleotide sequence represented by SEQ ID NOS: 159683, 159684, 159685, 159686, 159687, 159688, 159689, 159690, 159691, 159692, 159693 were selected from the ORFs having the nucleotide sequence represented by SEQ ID NO: 82209.

The probes having the nucleotide sequence represented by SEQ ID NOS: 159694, 159695, 159696, 159697, 159698, 159699, 159700, 159701, 159702, 159703, 159704 were selected from the ORFs having the nucleotide sequence represented by SEQ ID NO: 82210.

The probes having the nucleotide sequence represented by SEQ ID NOS: 159705, 159706, 159707, 159708, 159709, 159710, 159711, 159712, 159713, 159714, 159715 were selected from the ORFs having the nucleotide sequence represented by SEQ ID NO: 82211.

The probes having the nucleotide sequence represented by SEQ ID NOS: 159716, 159717, 159718, 159719, 159720, 159721, 159722, 159723, 159724, 159725, 159726 were selected from the ORFs having the nucleotide sequence represented by SEQ ID NO: 82212.

The probes having the nucleotide sequence represented by SEQ ID NOS: 159727, 159728, 159729, 159730, 159731, 159732, 159733, 159734, 159735, 159736, 159737 were selected from the ORFs having the nucleotide sequence represented by SEQ ID NO: 82213.

The probes having the nucleotide sequence represented by SEQ ID NOS: 159738, 159739, 159740, 159741, 159742, 159743, 159744, 159745, 159746, 159747, 159748, 159749, 159750, 159751, 159752, 159753, 159754, 159755, 159756, 159757, 159758, 159759 were selected from the ORFs having the nucleotide sequence represented by SEQ ID NO: 82214.

The probes having the nucleotide sequence represented by SEQ ID NOS: 159760, 159761, 159762, 159763, 159764, 159765, 159766, 159767, 159768, 159769, 159770 were selected from the ORFs having the nucleotide sequence represented by SEQ ID NO: 82215.

The probes having the nucleotide sequence represented by SEQ ID NOS: 159771, 159772, 159773, 159774, 159775, 159776, 159777, 159778, 159779, 159780, 159781 were selected from the ORFs having the nucleotide sequence represented by SEQ ID NO: 82216.

The probes having the nucleotide sequence represented by SEQ ID NOS: 159782, 159783, 159784, 159785, 159786, 159787, 159788, 159789, 159790, 159791, 159792 were selected from the ORFs having the nucleotide sequence represented by SEQ ID NO: 82217.

The probes having the nucleotide sequence represented by SEQ ID NOS: 159793, 159794, 159795, 159796, 159797, 159798, 159799, 159800, 159801, 159802, 159803 were selected from the ORFs having the nucleotide sequence represented by SEQ ID NO: 82218.

The probes having the nucleotide sequence represented by SEQ ID NOS: 159804, 159805, 159806, 159807, 159808, 159809, 159810, 159811, 159812, 159813, 159814 were selected from the ORFs having the nucleotide sequence represented by SEQ ID NO: 82219.

The probes having the nucleotide sequence represented by SEQ ID NOS: 159815, 159816, 159817, 159818, 159819, 159820, 159821, 159822, 159823, 159824, 159825 were selected from the ORFs having the nucleotide sequence represented by SEQ ID NO: 82220.

The probes having the nucleotide sequence represented by SEQ ID NOS: 159826, 159827, 159828, 159829, 159830, 159831, 159832, 159833, 159834, 159835, 159836 were selected from the ORFs having the nucleotide sequence represented by SEQ ID NO: 82221.

The probes having the nucleotide sequence represented by SEQ ID NOS: 159837, 159838, 159839, 159840, 159841, 159842, 159843, 159844, 159845, 159846, 159847 were selected from the ORFs having the nucleotide sequence represented by SEQ ID NO: 82222.

The probes having the nucleotide sequence represented by SEQ ID NOS: 159848, 159849, 159850, 159851, 159852, 159853, 159854, 159855, 159856, 159857, 159858 were selected from the ORFs having the nucleotide sequence represented by SEQ ID NO: 82223.

The probes having the nucleotide sequence represented by SEQ ID NOS: 159859, 159860, 159861, 159862, 159863, 159864, 159865, 159866, 159867, 159868, 159869 were selected from the ORFs having the nucleotide sequence represented by SEQ ID NO: 82224.

The probes having the nucleotide sequence represented by SEQ ID NOS: 159870, 159871, 159872, 159873, 159874, 159875, 159876, 159877, 159878, 159879, 159880 were selected from the ORFs having the nucleotide sequence represented by SEQ ID NO: 82225.

The probes having the nucleotide sequence represented by SEQ ID NOS: 159881, 159882, 159883, 159884, 159885, 159886, 159887, 159888, 159889, 159890, 159891 were selected from the ORFs having the nucleotide sequence represented by SEQ ID NO: 82226.

The probes having the nucleotide sequence represented by SEQ ID NOS: 159892, 159893, 159894, 159895, 159896, 159897, 159898, 159899, 159900, 159901, 159902 were selected from the ORFs having the nucleotide sequence represented by SEQ ID NO: 82227.

The probes having the nucleotide sequence represented by SEQ ID NOS: 159903, 159904, 159905, 159906, 159907, 159908, 159909, 159910, 159911, 159912, 159913 were selected from the ORFs having the nucleotide sequence represented by SEQ ID NO: 82228.

The probes having the nucleotide sequence represented by SEQ ID NOS: 159914, 159915, 159916, 159917, 159918, 159919, 159920, 159921, 159922, 159923, 159924 were selected from the ORFs having the nucleotide sequence represented by SEQ ID NO: 82229.

The probes having the nucleotide sequence represented by SEQ ID NOS: 159925, 159926, 159927, 159928, 159929, 159930, 159931, 159932, 159933, 159934, 159935 were selected from the ORFs having the nucleotide sequence represented by SEQ ID NO: 82230.

The probes having the nucleotide sequence represented by SEQ ID NOS: 159936, 159937, 159938, 159939, 159940, 159941, 159942, 159943, 159944, 159945, 159946 were selected from the ORFs having the nucleotide sequence represented by SEQ ID NO: 82231.

The probes having the nucleotide sequence represented by SEQ ID NOS: 159947, 159948, 159949, 159950, 159951, 159952, 159953, 159954, 159955, 159956, 159957 were selected from the ORFs having the nucleotide sequence represented by SEQ ID NO: 82232.

The probes having the nucleotide sequence represented by SEQ ID NOS: 159958, 159959, 159960, 159961, 159962, 159963, 159964, 159965, 159966, 159967, 159968 were selected from the ORFs having the nucleotide sequence represented by SEQ ID NO: 82233.

The probes having the nucleotide sequence represented by SEQ ID NOS: 159969, 159970, 159971, 159972, 159973, 159974, 159975, 159976, 159977, 159978, 159979 were selected from the ORFs having the nucleotide sequence represented by SEQ ID NO: 82234.

The probes having the nucleotide sequence represented by SEQ ID NOS: 159980, 159981, 159982, 159983, 159984, 159985, 159986, 159987, 159988, 159989, 159990 were selected from the ORFs having the nucleotide sequence represented by SEQ ID NO: 82235.

The probes having the nucleotide sequence represented by SEQ ID NOS: 159991, 159992, 159993, 159994, 159995, 159996, 159997, 159998, 159999, 160000, 160001 were selected from the ORFs having the nucleotide sequence represented by SEQ ID NO: 82236.

The probes having the nucleotide sequence represented by SEQ ID NOS: 160002, 160003, 160004, 160005, 160006, 160007, 160008, 160009, 160010, 160011, 160012 were selected from the ORFs having the nucleotide sequence represented by SEQ ID NO: 82237.

The probes having the nucleotide sequence represented by SEQ ID NOS: 160013, 160014, 160015, 160016, 160017, 160018, 160019, 160020, 160021, 160022, 160023 were selected from the ORFs having the nucleotide sequence represented by SEQ ID NO: 82238.

The probes having the nucleotide sequence represented by SEQ ID NOS: 160024, 160025, 160026, 160027, 160028, 160029, 160030, 160031, 160032, 160033, 160034 were selected from the ORFs having the nucleotide sequence represented by SEQ ID NO: 82239.

The probes having the nucleotide sequence represented by SEQ ID NOS: 160035, 160036, 160037, 160038, 160039, 160040, 160041, 160042, 160043, 160044, 160045 were selected from the ORFs having the nucleotide sequence represented by SEQ ID NO: 82240.

The probes having the nucleotide sequence represented by SEQ ID NOS: 160046, 160047, 160048, 160049, 160050, 160051, 160052, 160053, 160054, 160055, 160056 were selected from the ORFs having the nucleotide sequence represented by SEQ ID NO: 82241.

The probes having the nucleotide sequence represented by SEQ ID NOS: 160057, 160058, 160059, 160060, 160061, 160062, 160063, 160064, 160065, 160066, 160067 were selected from the ORFs having the nucleotide sequence represented by SEQ. ID NO: 82242.

The probes having the nucleotide sequence represented by SEQ ID NOS: 160068, 160069, 160070, 160071, 160072, 160073, 160074, 160075, 160076, 160077, 160078 were selected from the ORFs having the nucleotide sequence represented by SEQ ID NO: 82243.

The probes having the nucleotide sequence represented by SEQ ID NOS: 160079, 160080, 160081, 160082, 160083, 160084, 160085, 160086, 160087, 160088, 160089 were selected from the ORFs having the nucleotide sequence represented by SEQ ID NO: 82244.

The probes having the nucleotide sequence represented by SEQ ID NOS: 160090, 160091, 160092, 160093, 160094, 160095, 160096, 160097, 160098, 160099, 160100 were selected from the ORFs having the nucleotide sequence represented by SEQ ID NO: 82245.

The probes having the nucleotide sequence represented by SEQ ID NOS: 160101, 160102, 160103, 160104, 160105, 160106, 160107, 160108, 160109, 160110, 160111 were selected from the ORFs having the nucleotide sequence represented by SEQ ID NO: 82246.

The probes having the nucleotide sequence represented by SEQ ID NOS: 160112, 160113, 160114, 160115, 160116, 160117, 160118, 160119, 160120, 160121, 160122 were selected from the ORFs having the nucleotide sequence represented by SEQ ID NO: 82247.

The probes having the nucleotide sequence represented by SEQ ID NOS: 160123, 160124, 160125, 160126, 160127, 160128, 160129, 160130, 160131, 160132, 160133 were selected from the ORFs having the nucleotide sequence represented by SEQ ID NO: 82248.

The probes having the nucleotide sequence represented by SEQ ID NOS: 160134, 160135, 160136, 160137, 160138, 160139, 160140, 160141, 160142, 160143, 160144 were selected from the ORFs having the nucleotide sequence represented by SEQ ID NO: 82249.

The probes having the nucleotide sequence represented by SEQ ID NOS: 160145, 160146, 160147, 160148, 160149, 160150, 160151, 160152, 160153, 160154, 160155 were selected from the ORFs having the nucleotide sequence represented by SEQ ID NO: 82250.

The probes having the nucleotide sequence represented by SEQ ID NOS: 160156, 160157, 160158, 160159, 160160, 160161, 160162, 160163, 160164, 160165, 160166 were selected from the ORFs having the nucleotide sequence represented by SEQ ID NO: 82251.

The probes having the nucleotide sequence represented by SEQ ID NOS: 160167, 160168, 160169, 160170, 160171, 160172, 160173, 160174, 160175, 160176, 160177 were selected from the ORFs having the nucleotide sequence represented by SEQ ID NO: 82252.

The probes having the nucleotide sequence represented by SEQ ID NOS: 160178, 160179, 160180, 160181, 160182, 160183, 160184, 160185, 160186, 160187, 160188 were selected from the ORFs having the nucleotide sequence represented by SEQ ID NO: 82253.

The probes having the nucleotide sequence represented by SEQ ID NOS: 160189, 160190, 160191, 160192, 160193, 160194, 160195, 160196, 160197, 160198, 160199 were selected from the ORFs having the nucleotide sequence represented by SEQ ID NO: 82254.

The probes having the nucleotide sequence represented by SEQ ID NOS: 160200, 160201, 160202, 160203, 160204, 160205, 160206, 160207, 160208, 160209, 160210 were selected from the ORFs having the nucleotide sequence represented by SEQ ID NO: 82255.

The probes having the nucleotide sequence represented by SEQ ID NOS: 160211, 160212, 160213, 160214, 160215, 160216, 160217, 160218, 160219, 160220, 160221 were selected from the ORFs having the nucleotide sequence represented by SEQ ID NO: 82256.

The probes having the nucleotide sequence represented by SEQ ID NOS: 160222, 160223, 160224, 160225, 160226, 160227, 160228, 160229, 160230, 160231, 160232 were selected from the ORFs having the nucleotide sequence represented by SEQ ID NO: 82257.

The probes having the nucleotide sequence represented by SEQ ID NOS: 160233, 160234, 160235, 160236, 160237, 160238, 160239, 160240, 160241, 160242, 160243 were selected from the ORFs having the nucleotide sequence represented by SEQ ID NO: 82258.

The probes having the nucleotide sequence represented by SEQ ID NOS: 160244, 160245, 160246, 160247, 160248, 160249, 160250, 160251, 160252, 160253, 160254 were selected from the ORFs having the nucleotide sequence represented by SEQ ID NO: 82259.

The probes having the nucleotide sequence represented by SEQ ID NOS: 160255, 160256, 160257, 160258, 160259, 160260, 160261, 160262, 160263, 160264, 160265 were selected from the ORFs having the nucleotide sequence represented by SEQ ID NO: 82260.

The probes having the nucleotide sequence represented by SEQ ID NOS: 160266, 160267, 160268, 160269, 160270, 160271, 160272, 160273, 160274, 160275, 160276 were selected from the ORFs having the nucleotide sequence represented by SEQ ID NO: 82261.

The probes having the nucleotide sequence represented by SEQ ID NOS: 160277, 160278, 160279, 160280, 160281, 160282, 160283, 160284, 160285, 160286, 160287 were selected from the ORFs having the nucleotide sequence represented by SEQ ID NO: 82262.

The probes having the nucleotide sequence represented by SEQ ID NOS: 160288, 160289, 160290, 160291, 160292, 160293, 160294, 160295, 160296, 160297, 160298 were selected from the ORFs having the nucleotide sequence represented by SEQ ID NO: 82263.

The probes having the nucleotide sequence represented by SEQ ID NOS: 160299, 160300, 160301, 160302, 160303, 160304, 160305, 160306, 160307, 160308, 160309 were selected from the ORFs having the nucleotide sequence represented by SEQ ID NO: 82264.

The probes having the nucleotide sequence represented by SEQ ID NOS: 160310, 160311, 160312, 160313, 160314, 160315, 160316, 160317, 160318, 160319, 160320 were selected from the ORFs having the nucleotide sequence represented by SEQ ID NO: 82265.

The probes having the nucleotide sequence represented by SEQ ID NOS: 160321, 160322, 160323, 160324, 160325, 160326, 160327, 160328, 160329, 160330, 160331 were selected from the ORFs having the nucleotide sequence represented by SEQ ID NO: 82266.

The probes having the nucleotide sequence represented by SEQ ID NOS: 160332, 160333, 160334, 160335, 160336, 160337, 160338, 160339, 160340, 160341, 160342 were selected from the ORFs having the nucleotide sequence represented by SEQ ID NO: 82267.

The probes having the nucleotide sequence represented by SEQ ID NOS: 160343, 160344, 160345, 160346, 160347, 160348, 160349, 160350, 160351, 160352, 160353 were selected from the ORFs having the nucleotide sequence represented by SEQ ID NO: 82268.

The probes having the nucleotide sequence represented by SEQ ID NOS: 160354, 160355, 160356, 160357, 160358, 160359, 160360, 160361, 160362, 160363, 160364 were selected from the ORFs having the nucleotide sequence represented by SEQ ID NO: 82269.

The probes having the nucleotide sequence represented by SEQ ID NOS: 160365, 160366, 160367, 160368, 160369, 160370, 160371, 160372, 160373, 160374, 160375 were selected from the ORFs having the nucleotide sequence represented by SEQ ID NO: 82270.

The probes having the nucleotide sequence represented by SEQ ID NOS: 160376, 160377, 160378, 160379, 160380, 160381, 160382, 160383, 160384, 160385, 160386 were selected from the ORFs having the nucleotide sequence represented by SEQ ID NO: 82271.

The probes having the nucleotide sequence represented by SEQ ID NOS: 160387, 160388, 160389, 160390, 160391, 160392, 160393, 160394, 160395, 160396, 160397 were selected from the ORFs having the nucleotide sequence represented by SEQ ID NO: 82272.

The probes having the nucleotide sequence represented by SEQ ID NOS: 160398, 160399, 160400, 160401, 160402, 160403, 160404, 160405, 160406, 160407, 160408 were selected from the ORFs having the nucleotide sequence represented by SEQ ID NO: 82273.

The probes having the nucleotide sequence represented by SEQ ID NOS: 160409, 160410, 160411, 160412, 160413, 160414, 160415, 160416, 160417, 160418, 160419 were selected from the ORFs having the nucleotide sequence represented by SEQ ID NO: 82274.

The probes having the nucleotide sequence represented by SEQ ID NOS: 160420, 160421, 160422, 160423, 160424, 160425, 160426, 160427, 160428, 160429, 160430 were selected from the ORFs having the nucleotide sequence represented by SEQ ID NO: 82275.

The probes having the nucleotide sequence represented by SEQ ID NOS: 160431, 160432, 160433, 160434, 160435, 160436, 160437, 160438, 160439, 160440, 160441 were selected from the ORFs having the nucleotide sequence represented by SEQ ID NO: 82276.

The probes having the nucleotide sequence represented by SEQ ID NOS: 160442, 160443, 160444, 160445, 160446, 160447, 160448, 160449, 160450, 160451, 160452 were selected from the ORFs having the nucleotide sequence represented by SEQ ID NO: 82277.

The probes having the nucleotide sequence represented by SEQ ID NOS: 160453, 160454, 160455, 160456, 160457, 160458, 160459, 160460, 160461, 160462, 160463 were selected from the ORFs having the nucleotide sequence represented by SEQ ID NO: 82278.

The probes having the nucleotide sequence represented by SEQ ID NOS: 160464, 160465, 160466, 160467, 160468, 160469, 160470, 160471, 160472, 160473, 160474 were selected from the ORFs having the nucleotide sequence represented by SEQ ID NO: 82279.

The probes having the nucleotide sequence represented by SEQ ID NOS: 160475, 160476, 160477, 160478, 160479, 160480, 160481, 160482, 160483, 160484, 160485 were selected from the ORFs having the nucleotide sequence represented by SEQ ID NO: 82280.

The probes having the nucleotide sequence represented by SEQ ID NOS: 160486, 160487, 160488, 160489, 160490, 160491, 160492, 160493, 160494, 160495, 160496 were selected from the ORFs having the nucleotide sequence represented by SEQ ID NO: 82281.

The probes having the nucleotide sequence represented by SEQ ID NOS: 160497, 160498, 160499, 160500, 160501, 160502, 160503, 160504, 160505, 160506, 160507 were selected from the ORFs having the nucleotide sequence represented by SEQ ID NO: 82282.

The probes having the nucleotide sequence represented by SEQ ID NOS: 160508, 160509, 160510, 160511, 160512, 160513, 160514, 160515, 160516, 160517, 160518 were selected from the ORFs having the nucleotide sequence represented by SEQ ID NO: 82283.

The probes having the nucleotide sequence represented by SEQ ID NOS: 160519, 160520, 160521, 160522, 160523, 160524, 160525, 160526, 160527, 160528, 160529 were selected from the ORFs having the nucleotide sequence represented by SEQ ID NO: 82284.

The probes having the nucleotide sequence represented by SEQ ID NOS: 160530, 160531, 160532, 160533, 160534, 160535, 160536, 160537, 160538, 160539, 160540 were selected from the ORFs having the nucleotide sequence represented by SEQ ID NO: 82285.

The probes having the nucleotide sequence represented by SEQ ID NOS: 160541, 160542, 160543, 160544, 160545, 160546, 160547, 160548, 160549, 160550, 160551 were selected from the ORFs having the nucleotide sequence represented by SEQ ID NO: 82286.

The probes having the nucleotide sequence represented by SEQ ID NOS: 160552, 160553, 160554, 160555, 160556, 160557, 160558, 160559, 160560, 160561, 160562 were selected from the ORFs having the nucleotide sequence represented by SEQ ID NO: 82287.

The probes having the nucleotide sequence represented by SEQ ID NOS: 160563, 160564, 160565, 160566, 160567, 160568, 160569, 160570, 160571, 160572, 160573 were selected from the ORFs having the nucleotide sequence represented by SEQ ID NO: 82288.

The probes having the nucleotide sequence represented by SEQ ID NOS: 160574, 160575, 160576, 160577, 160578, 160579, 160580, 160581, 160582, 160583, 160584 were selected from the ORFs having the nucleotide sequence represented by SEQ ID NO: 82289.

The probes having the nucleotide sequence represented by SEQ ID NOS: 160585, 160586, 160587, 160588, 160589, 160590, 160591, 160592, 160593, 160594, 160595 were selected from the ORFs having the nucleotide sequence represented by SEQ ID NO: 82290.

The probes having the nucleotide sequence represented by SEQ ID NOS: 160596, 160597, 160598, 160599, 160600, 160601, 160602, 160603, 160604, 160605, 160606 were selected from the ORFs having the nucleotide sequence represented by SEQ ID NO: 82291.

The probes having the nucleotide sequence represented by SEQ ID NOS: 160607, 160608, 160609, 160610, 160611, 160612, 160613, 160614, 160615, 160616, 160617 were selected from the ORFs having the nucleotide sequence represented by SEQ ID NO: 82292.

The probes having the nucleotide sequence represented by SEQ ID NOS: 160618, 160619, 160620, 160621, 160622, 160623, 160624, 160625, 160626, 160627, 160628 were selected from the ORFs having the nucleotide sequence represented by SEQ ID NO: 82293.

The probes having the nucleotide sequence represented by SEQ ID NOS: 160629, 160630, 160631, 160632, 160633, 160634, 160635, 160636, 160637, 160638, 160639 were selected from the ORFs having the nucleotide sequence represented by SEQ ID NO: 82294.

The probes having the nucleotide sequence represented by SEQ ID NOS: 160640, 160641, 160642, 160643, 160644, 160645, 160646, 160647, 160648, 160649, 160650 were selected from the ORFs having the nucleotide sequence represented by SEQ ID NO: 82295.

The probes having the nucleotide sequence represented by SEQ ID NOS: 160651,. 160652, 160653, 160654, 160655, 160656, 160657, 160658, 160659, 160660, 160661 were selected from the ORFs having the nucleotide sequence represented by SEQ ID NO: 82296.

The probes having the nucleotide sequence represented by SEQ ID NOS: 160662, 160663, 160664, 160665, 160666, 160667, 160668, 160669, 160670, 160671, 160672 were selected from the ORFs having the nucleotide sequence represented by SEQ ID NO: 82297.

The probes having the nucleotide sequence represented by SEQ ID NOS: 160673, 160674, 160675, 160676, 160677, 160678, 160679, 160680, 160681, 160682, 160683 were selected from the ORFs having the nucleotide sequence represented by SEQ ID NO: 82298.

The probes having the nucleotide sequence represented by SEQ ID NOS: 160684, 160685, 160686, 160687, 160688, 160689, 160690, 160691, 160692, 160693, 160694 were selected from the ORFs having the nucleotide sequence represented by SEQ ID NO: 82299.

The probes having the nucleotide sequence represented by SEQ ID NOS: 160695, 160696, 160697, 160698, 160699, 160700, 160701, 160702, 160703, 160704, 160705 were selected from the ORFs having the nucleotide sequence represented by SEQ ID NO: 82300.

The probes having the nucleotide sequence represented by SEQ ID NOS: 160706, 160707, 160708, 160709, 160710, 160711, 160712, 160713, 160714, 160715, 160716 were selected from the ORFs having the nucleotide sequence represented by SEQ ID NO: 82301.

The probes having the nucleotide sequence represented by SEQ ID NOS: 160717, 160718, 160719, 160720, 160721, 160722, 160723, 160724, 160725, 160726, 160727 were selected from the ORFs having the nucleotide sequence represented by SEQ ID NO: 82302.

The probes having the nucleotide sequence represented by SEQ ID NOS: 160728, 160729, 160730, 160731, 160732, 160733, 160734, 160735, 160736, 160737, 160738 were selected from the ORFs having the nucleotide sequence represented by SEQ ID NO: 82303.

The probes having the nucleotide sequence represented by SEQ ID NOS: 160739, 160740, 160741, 160742, 160743, 160744, 160745, 160746, 160747, 160748, 160749 were selected from the ORFs having the nucleotide sequence represented by SEQ ID NO: 82304.

The probes having the nucleotide sequence represented by SEQ ID NOS: 160750, 160751, 160752, 160753, 160754, 160755, 160756, 160757, 160758, 160759, 160760 were selected from the ORFs having the nucleotide sequence represented by SEQ ID NO: 82305.

The probes having the nucleotide sequence represented by SEQ ID NOS: 160761, 160762, 160763, 160764, 160765, 160766, 160767, 160768, 160769, 160770, 160771 were selected from the ORFs having the nucleotide sequence represented by SEQ ID NO: 82306.

The probes having the nucleotide sequence represented by SEQ ID NOS: 160772, 160773, 160774, 160775, 160776, 160777, 160778, 160779, 160780, 160781, 160782 were selected from the ORFs having the nucleotide sequence represented by SEQ ID NO: 82307.

The probes having the nucleotide sequence represented by SEQ ID NOS: 160783, 160784, 160785, 160786, 160787, 160788, 160789, 160790, 160791, 160792, 160793 were selected from the ORFs having the nucleotide sequence represented by SEQ ID NO: 82308.

The probes having the nucleotide sequence represented by SEQ ID NOS: 160794, 160795, 160796, 160797, 160798, 160799, 160800, 160801, 160802, 160803, 160804 were selected from the ORFs having the nucleotide sequence represented by SEQ ID NO: 82309.

The probes having the nucleotide sequence represented by SEQ ID NOS: 160805, 160806, 160807, 160808, 160809, 160810, 160811, 160812, 160813, 160814, 160815 were selected from the ORFs having the nucleotide sequence represented by SEQ ID NO: 82310.

The probes having the nucleotide sequence represented by SEQ ID NOS: 160816, 160817, 160818, 160819, 160820, 160821, 160822, 160823, 160824, 160825, 160826 were selected from the ORFs having the nucleotide sequence represented by SEQ ID NO: 82311.

The probes having the nucleotide sequence represented by SEQ ID NOS: 160827, 160828, 160829, 160830, 160831, 160832, 160833, 160834, 160835, 160836, 160837 were selected from the ORFs having the nucleotide sequence represented by SEQ ID NO: 82312.

The probes having the nucleotide sequence represented by SEQ ID NOS: 160838, 160839, 160840, 160841, 160842, 160843, 160844, 160845, 160846, 160847, 160848 were selected from the ORFs having the nucleotide sequence represented by SEQ ID NO: 82313.

The probes having the nucleotide sequence represented by SEQ ID NOS: 160849, 160850, 160851, 160852, 160853, 160854, 160855, 160856, 160857, 160858, 160859 were selected from the ORFs having the nucleotide sequence represented by SEQ ID NO: 82314.

The probes having the nucleotide sequence represented by SEQ ID NOS: 160860, 160861, 160862, 160863, 160864, 160865, 160866, 160867, 160868, 160869, 160870 were selected from the ORFs having the nucleotide sequence represented by SEQ ID NO: 82315.

The probes having the nucleotide sequence represented by SEQ ID NOS: 160871, 160872, 160873, 160874, 160875, 160876, 160877, 160878, 160879, 160880, 160881 were selected from the ORFs having the nucleotide sequence represented by SEQ ID NO: 82316.

The probes having the nucleotide sequence represented by SEQ ID NOS: 160882, 160883, 160884, 160885, 160886, 160887, 160888, 160889, 160890, 160891, 160892 were selected from the ORFs having the nucleotide sequence represented by SEQ ID NO: 82317.

The probes having the nucleotide sequence represented by SEQ ID NOS: 160893, 160894, 160895, 160896, 160897, 160898, 160899, 160900, 160901, 160902, 160903 were selected from the ORFs having the nucleotide sequence represented by SEQ ID NO: 82318.

The probes having the nucleotide sequence represented by SEQ ID NOS: 160904, 160905, 160906, 160907, 160908, 160909, 160910, 160911, 160912, 160913, 160914 were selected from the ORFs having the nucleotide sequence represented by SEQ ID NO: 82319.

The probes having the nucleotide sequence represented by SEQ ID NOS: 160915, 160916, 160917, 160918, 160919, 160920, 160921, 160922, 160923, 160924, 160925 were selected from the ORFs having the nucleotide sequence represented by SEQ ID NO: 82320.

The probes having the nucleotide sequence represented by SEQ ID NOS: 160926, 160927, 160928, 160929, 160930, 160931, 160932, 160933, 160934, 160935, 160936 were selected from the ORFs having the nucleotide sequence represented by SEQ ID NO: 82321.

The probes having the nucleotide sequence represented by SEQ ID NOS: 160937, 160938, 160939, 160940, 160941, 160942, 160943, 160944, 160945, 160946, 160947 were selected from the ORFs having the nucleotide sequence represented by SEQ ID NO: 82322.

The probes having the nucleotide sequence represented by SEQ ID NOS: 160948, 160949, 160950, 160951, 160952, 160953, 160954, 160955, 160956, 160957, 160958 were selected from the ORFs having the nucleotide sequence represented by SEQ ID NO: 82323.

The probes having the nucleotide sequence represented by SEQ ID NOS: 160959, 160960, 160961, 160962, 160963, 160964, 160965, 160966, 160967, 160968, 160969 were selected from the ORFs having the nucleotide sequence represented by SEQ ID NO: 82324.

The probes having the nucleotide sequence represented by SEQ ID NOS: 160970, 160971, 160972, 160973, 160974, 160975, 160976, 160977, 160978, 160979, 160980 were selected from the ORFs having the nucleotide sequence represented by SEQ ID NO: 82325.

The probes having the nucleotide sequence represented by SEQ ID NOS: 160981, 160982, 160983, 160984, 160985, 160986, 160987, 160988, 160989, 160990, 160991 were selected from the ORFs having the nucleotide sequence represented by SEQ ID NO: 82326.

The probes having the nucleotide sequence represented by SEQ ID NOS: 160992, 160993, 160994, 160995, 160996, 160997, 160998, 160999, 161000, 161001, 161002 were selected from the ORFs having the nucleotide sequence represented by SEQ ID NO: 82327.

The probes having the nucleotide sequence represented by SEQ ID NOS: 161003, 161004, 161005, 161006, 161007, 161008, 161009, 161010, 161011, 161012, 161013 were selected from the ORFs having the nucleotide sequence represented by SEQ ID NO: 82328.

The probes having the nucleotide sequence represented by SEQ ID NOS: 161014, 161015, 161016, 161017, 161018, 161019, 161020, 161021, 161022, 161023, 161024 were selected from the ORFs having the nucleotide sequence represented by SEQ ID NO: 82329.

The probes having the nucleotide sequence represented by SEQ ID NOS: 161025, 161026, 161027, 161028, 161029, 161030, 161031, 161032, 161033, 161034, 161035 were selected from the ORFs having the nucleotide sequence represented by SEQ ID NO: 82330.

The probes having the nucleotide sequence represented by SEQ ID NOS: 161036, 161037, 161038, 161039, 161040, 161041, 161042, 161043, 161044, 161045, 161046, 161047, 161048, 161049, 161050, 161051, 161052, 161053, 161054, 161055, 161056, 161057 were selected from the ORFs having the nucleotide sequence represented by SEQ ID NO: 82331.

The probes having the nucleotide sequence represented by SEQ ID NOS: 161058, 161059, 161060, 161061, 161062, 161063, 161064, 161065, 161066, 161067, 161068 were selected from the ORFs having the nucleotide sequence represented by SEQ ID NO: 82332.

The probes having the nucleotide sequence represented by SEQ ID NOS: 161069, 161070, 161071, 161072, 161073, 161074, 161075, 161076, 161077, 161078, 161079 were selected from the ORFs having the nucleotide sequence represented by SEQ ID NO: 82333.

The probes having the nucleotide sequence represented by SEQ ID NOS: 161080, 161081, 161082, 161083, 161084, 161085, 161086, 161087, 161088, 161089, 161090 were selected from the ORFs having the nucleotide sequence represented by SEQ ID NO: 82334.

The probes having the nucleotide sequence represented by SEQ ID NOS: 161091, 161092, 161093, 161094, 161095, 161096, 161097, 161098, 161099, 161100, 161101 were selected from the ORFs having the nucleotide sequence represented by SEQ ID NO: 82335.

The probes having the nucleotide sequence represented by SEQ ID NOS: 161102, 161103, 161104, 161105, 161106, 161107, 161108, 161109, 161110, 161111, 161112 were selected from the ORFs having the nucleotide sequence represented by SEQ ID NO: 82336.

The probes having the nucleotide sequence represented by SEQ ID NOS: 161113, 161114, 161115, 161116, 161117, 161118, 161119, 161120, 161121, 161122, 161123, 161124, 161125, 161126, 161127, 161128, 161129, 161130, 161131, 161132, 161133, 161134 were selected from the ORFs having the nucleotide sequence represented by SEQ ID NO: 82337.

The probes having the nucleotide sequence represented by SEQ ID NOS: 161135, 161136, 161137, 161138, 161139, 161140, 161141, 161142, 161143, 161144, 161145 were selected from the ORFs having the nucleotide sequence represented by SEQ ID NO: 82338.

The probes having the nucleotide sequence represented by SEQ ID NOS: 161146, 161147, 161148, 161149, 161150, 161151, 161152, 161153, 161154, 161155, 161156 were selected from the ORFs having the nucleotide sequence represented by SEQ ID NO: 82339.

The probes having the nucleotide sequence represented by SEQ ID NOS: 161157, 161158, 161159, 161160, 161161, 161162, 161163, 161164, 161165, 161166, 161167 were selected from the ORFs having the nucleotide sequence represented by SEQ ID NO: 82340.

The probes having the nucleotide sequence represented by SEQ ID NOS: 161168, 161169, 161170, 161171, 161172, 161173, 161174, 161175, 161176, 161177, 161178 were selected from the ORFs having the nucleotide sequence represented by SEQ ID NO: 82341.

The probes having the nucleotide sequence represented by SEQ ID NOS: 161179, 161180, 161181, 161182, 161183, 161184, 161185, 161186, 161187, 161188, 161189 were selected from the ORFs having the nucleotide sequence represented by SEQ ID NO: 82342.

The probes having the nucleotide sequence represented by SEQ ID NOS: 161190, 161191, 161192, 161193, 161194, 161195, 161196, 161197, 161198, 161199, 161200 were selected from the ORFs having the nucleotide sequence represented by SEQ ID NO: 82343.

The probes having the nucleotide sequence represented by SEQ ID NOS: 161201, 161202, 161203, 161204, 161205, 161206, 161207, 161208, 161209, 161210, 161211 were selected from the ORFs having the nucleotide sequence represented by SEQ ID NO: 82344.

The probes having the nucleotide sequence represented by SEQ ID NOS: 161212, 161213, 161214, 161215, 161216, 161217, 161218, 161219, 161220, 161221, 161222 were selected from the ORFs having the nucleotide sequence represented by SEQ ID NO: 82345.

The probes having the nucleotide sequence represented by SEQ ID NOS: 161223, 161224, 161225, 161226, 161227, 161228, 161229, 161230, 161231, 161232, 161233 were selected from the ORFs having the nucleotide sequence represented by SEQ ID NO: 82346.

The probes having the nucleotide sequence represented by SEQ ID NOS: 161234, 161235, 161236, 161237, 161238, 161239, 161240, 161241, 161242, 161243, 161244 were selected from the ORFs having the nucleotide sequence represented by SEQ ID NO: 82347.

The probes having the nucleotide sequence represented by SEQ ID NOS: 161245, 161246, 161247, 161248, 161249, 161250, 161251, 161252, 161253, 161254, 161255 were selected from the ORFs having the nucleotide sequence represented by SEQ ID NO: 82348.

The probes having the nucleotide sequence represented by SEQ ID NOS: 161256, 161257, 161258, 161259, 161260, 161261, 161262, 161263, 161264, 161265, 161266 were selected from the ORFs having the nucleotide sequence represented by SEQ ID NO: 82349.

The probes having the nucleotide sequence represented by SEQ ID NOS: 161267, 161268, 161269, 161270, 161271, 161272, 161273, 161274, 161275, 161276, 161277 were selected from the ORFs having the nucleotide sequence represented by SEQ ID NO: 82350.

The probes having the nucleotide sequence represented by SEQ ID NOS: 161278, 161279, 161280, 161281, 161282, 161283, 161284, 161285, 161286, 161287, 161288 were selected from the ORFs having the nucleotide sequence represented by SEQ ID NO: 82351.

The probes having the nucleotide sequence represented by SEQ ID NOS: 161289, 161290, 161291, 161292, 161293, 161294, 161295, 161296, 161297, 161298, 161299, 161300, 161301, 161302, 161303, 161304, 161305, 161306, 161307, 161308, 161309, 161310 were selected from the ORFs having the nucleotide sequence represented by SEQ ID NO: 82352.

The probes having the nucleotide sequence represented by SEQ ID NOS: 161311, 161312, 161313, 161314, 161315, 161316, 161317, 161318, 161319, 161320, 161321 were selected from the ORFs having the nucleotide sequence represented by SEQ ID NO: 82353.

The probes having the nucleotide sequence represented by SEQ ID NOS: 161322, 161323, 161324, 161325, 161326, 161327, 161328, 161329, 161330, 161331, 161332 were selected from the ORFs having the nucleotide sequence represented by SEQ ID NO: 82354.

The probes having the nucleotide sequence represented by SEQ ID NOS: 161333, 161334, 161335, 161336, 161337, 161338, 161339, 161340, 161341, 161342, 161343 were selected from the ORFs having the nucleotide sequence represented by SEQ ID NO: 82355.

The probes having the nucleotide sequence represented by SEQ ID NOS: 161344, 161345, 161346, 161347, 161348, 161349, 161350, 161351, 161352, 161353, 161354 were selected from the ORFs having the nucleotide sequence represented by SEQ ID NO: 82356.

The probes having the nucleotide sequence represented by SEQ ID NOS: 161355, 161356, 161357, 161358, 161359, 161360, 161361, 161362, 161363 were selected from the ORFs having the nucleotide sequence represented by SEQ ID NO: 82357.

The probes having the nucleotide sequence represented by SEQ ID NOS: 161364, 161365, 161366, 161367, 161368, 161369, 161370, 161371, 161372, 161373, 161374 were selected from the ORFs having the nucleotide sequence represented by SEQ ID NO: 82358.

The probes having the nucleotide sequence represented by SEQ ID NOS: 161375, 161376, 161377, 161378, 161379, 161380, 161381, 161382, 161383, 161384, 161385 were selected from the ORFs having the nucleotide sequence represented by SEQ ID NO: 82359.

The probes having the nucleotide sequence represented by SEQ ID NOS: 161386, 161387, 161388, 161389, 161390, 161391, 161392, 161393, 161394, 161395, 161396 were selected from the ORFs having the nucleotide sequence represented by SEQ ID NO: 82360.

The probes having the nucleotide sequence represented by SEQ ID NOS: 161397, 161398, 161399, 161400, 161401, 161402, 161403, 161404, 161405, 161406, 161407 were selected from the ORFs having the nucleotide sequence represented by SEQ ID NO: 82361.

The probes having the nucleotide sequence represented by SEQ ID NOS: 161408, 161409, 161410, 161411, 161412, 161413, 161414, 161415, 161416, 161417, 161418, 161419, 161420, 161421, 161422, 161423, 161424, 161425, 161426, 161427, 161428, 161429 were selected from the ORFs having the nucleotide sequence represented by SEQ ID NO: 82362.

The probes having the nucleotide sequence represented by SEQ ID NOS: 161430, 161431, 161432, 161433, 161434, 161435, 161436, 161437, 161438, 161439, 161440 were selected from the ORFs having the nucleotide sequence represented by SEQ ID NO: 82363.

The probes having the nucleotide sequence represented by SEQ ID NOS: 161441, 161442, 161443, 161444, 161445, 161446, 161447, 161448, 161449, 161450, 161451 were selected from the ORFs having the nucleotide sequence represented by SEQ ID NO: 82364.

The probes having the nucleotide sequence represented by SEQ ID NOS: 161452, 161453, 161454, 161455, 161456, 161457, 161458, 161459, 161460, 161461, 161462 were selected from the ORFs having the nucleotide sequence represented by SEQ ID NO: 82365.

The probes having the nucleotide sequence represented by SEQ ID NOS: 161463, 161464, 161465, 161466, 161467, 161468, 161469, 161470, 161471, 161472, 161473 were selected from the ORFs having the nucleotide sequence represented by SEQ ID NO: 82366.

The probes having the nucleotide sequence represented by SEQ ID NOS: 161474, 161475, 161476, 161477, 161478, 161479, 161480, 161481, 161482, 161483, 161484 were selected from the ORFs having the nucleotide sequence represented by SEQ ID NO: 82367.

The probes having the nucleotide sequence represented by SEQ ID NOS: 161485, 161486, 161487, 161488, 161489, 161490, 161491, 161492, 161493, 161494, 161495 were selected from the ORFs having the nucleotide sequence represented by SEQ ID NO: 82368.

The probes having the nucleotide sequence represented by SEQ ID NOS: 161496, 161497, 161498, 161499, 161500, 161501, 161502, 161503, 161504, 161505, 161506 were selected from the ORFs having the nucleotide sequence represented by SEQ ID NO: 82369.

The probes having the nucleotide sequence represented by SEQ ID NOS: 161507, 161508, 161509, 161510, 161511, 161512, 161513, 161514, 161515, 161516, 161517 were selected from the ORFs having the nucleotide sequence represented by SEQ ID NO: 82370.

The probes having the nucleotide sequence represented by SEQ ID NOS: 161518, 161519, 161520, 161521, 161522, 161523, 161524, 161525, 161526, 161527, 161528 were selected from the ORFs having the nucleotide sequence represented by SEQ ID NO: 82371.

The probes having the nucleotide sequence represented by SEQ ID NOS: 161529, 161530, 161531, 161532, 161533, 161534, 161535, 161536, 161537, 161538, 161539 were selected from the ORFs having the nucleotide sequence represented by SEQ ID NO: 82372.

The probes having the nucleotide sequence represented by SEQ ID NOS: 161540, 161541, 161542, 161543, 161544, 161545, 161546, 161547, 161548, 161549, 161550 were selected from the ORFs having the nucleotide sequence represented by SEQ ID NO: 82373.

The probes having the nucleotide sequence represented by SEQ ID NOS: 161551, 161552, 161553, 161554, 161555, 161556, 161557, 161558, 161559, 161560, 161561 were selected from the ORFs having the nucleotide sequence represented by SEQ ID NO: 82374.

The probes having the nucleotide sequence represented by SEQ ID NOS: 161562, 161563, 161564, 161565, 161566, 161567, 161568, 161569, 161570, 161571, 161572 were selected from the ORFs having the nucleotide sequence represented by SEQ ID NO: 82375.

The probes having the nucleotide sequence represented by SEQ ID NOS: 161573, 161574, 161575, 161576, 161577, 161578, 161579, 161580, 161581, 161582, 161583 were selected from the ORFs having the nucleotide sequence represented by SEQ ID NO: 82376.

The probes having the nucleotide sequence represented by SEQ ID NOS: 161584, 161585, 161586, 161587, 161588, 161589, 161590, 161591, 161592, 161593, 161594 were selected from the ORFs having the nucleotide sequence represented by SEQ ID NO: 82377.

The probes having the nucleotide sequence represented by SEQ ID NOS: 161595, 161596, 161597, 161598, 161599, 161600, 161601, 161602, 161603, 161604, 161605 were selected from the ORFs having the nucleotide sequence represented by SEQ ID NO: 82378.

The probes having the nucleotide sequence represented by SEQ ID NOS: 161606, 161607, 161608, 161609, 161610, 161611, 161612, 161613, 161614, 161615, 161616 were selected from the ORFs having the nucleotide sequence represented by SEQ ID NO: 82379.

The probes having the nucleotide sequence represented by SEQ ID NOS: 161617, 161618, 161619, 161620, 161621, 161622, 161623, 161624, 161625, 161626, 161627 were selected from the ORFs having the nucleotide sequence represented by SEQ ID NO: 82380.

The probes having the nucleotide sequence represented by SEQ ID NOS: 161628, 161629, 161630, 161631, 161632, 161633, 161634, 161635, 161636, 161637, 161638 were selected from the ORFs having the nucleotide sequence represented by SEQ ID NO: 82381.

The probes having the nucleotide sequence represented by SEQ ID NOS: 161639, 161640, 161641, 161642, 161643, 161644, 161645, 161646, 161647, 161648, 161649 were selected from the ORFs having the nucleotide sequence represented by SEQ ID NO: 82382.

The probes having the nucleotide sequence represented by SEQ ID NOS: 161650, 161651, 161652, 161653, 161654, 161655, 161656, 161657, 161658, 161659, 161660 were selected from the ORFs having the nucleotide sequence represented by SEQ ID NO: 82383.

The probes having the nucleotide sequence represented by SEQ ID NOS: 161661, 161662, 161663, 161664, 161665, 161666, 161667, 161668, 161669, 161670, 161671 were selected from the ORFs having the nucleotide sequence represented by SEQ ID NO: 82384.

The probes having the nucleotide sequence represented by SEQ ID NOS: 161672, 161673, 161674, 161675, 161676, 161677, 161678, 161679, 161680, 161681, 161682 were selected from the ORFs having the nucleotide sequence represented by SEQ ID NO: 82385.

The probes having the nucleotide sequence represented by SEQ ID NOS: 161683, 161684, 161685, 161686, 161687, 161688, 161689, 161690, 161691, 161692, 161693 were selected from the ORFs having the nucleotide sequence represented by SEQ ID NO: 82386.

The probes having the nucleotide sequence represented by SEQ ID NOS: 161694, 161695, 161696, 161697, 161698, 161699, 161700, 161701, 161702, 161703, 161704 were selected from the ORFs having the nucleotide sequence represented by SEQ ID NO: 82387.

The probes having the nucleotide sequence represented by SEQ ID NOS: 161705, 161706, 161707, 161708, 161709, 161710, 161711, 161712, 161713, 161714, 161715 were selected from the ORFs having the nucleotide sequence represented by SEQ ID NO: 82388.

The probes having the nucleotide sequence represented by SEQ ID NOS: 161716, 161717, 161718, 161719, 161720, 161721, 161722, 161723, 161724, 161725, 161726 were selected from the ORFs having the nucleotide sequence represented by SEQ ID NO: 82389.

The probes having the nucleotide sequence represented by SEQ ID NOS: 161727, 161728, 161729, 161730, 161731, 161732, 161733, 161734, 161735, 161736, 161737 were selected from the ORFs having the nucleotide sequence represented by SEQ ID NO: 82390.

The probes having the nucleotide sequence represented by SEQ ID NOS: 161738, 161739, 161740, 161741, 161742, 161743, 161744, 161745, 161746, 161747, 161748 were selected from the ORFs having the nucleotide sequence represented by SEQ ID NO: 82391.

The probes having the nucleotide sequence represented by SEQ ID NOS: 161749, 161750, 161751, 161752, 161753, 161754, 161755, 161756, 161757, 161758, 161759 were selected from the ORFs having the nucleotide sequence represented by SEQ ID NO: 82392.

The probes having the nucleotide sequence represented by SEQ ID NOS: 161760, 161761, 161762, 161763, 161764, 161765, 161766, 161767, 161768, 161769, 161770 were selected from the ORFs having the nucleotide sequence represented by SEQ ID NO: 82393.

The probes having the nucleotide sequence represented by SEQ ID NOS: 161771, 161772, 161773, 161774, 161775, 161776, 161777, 161778, 161779, 161780, 161781, 161782, 161783, 161784, 161785, 161786, 161787, 161788, 161789, 161790, 161791, 161792 were selected from the ORFs having the nucleotide sequence represented by SEQ ID NO: 82394.

The probes having the nucleotide sequence represented by SEQ ID NOS: 161793, 161794, 161795, 161796, 161797, 161798, 161799, 161800, 161801, 161802, 161803 were selected from the ORFs having the nucleotide sequence represented by SEQ ID NO: 82395.

The probes having the nucleotide sequence represented by SEQ ID NOS: 161804, 161805, 161806, 161807, 161808, 161809, 161810, 161811, 161812, 161813, 161814 were selected from the ORFs having the nucleotide sequence represented by SEQ ID NO: 82396.

The probes having the nucleotide sequence represented by SEQ ID NOS: 161815, 161816, 161817, 161818, 161819, 161820, 161821, 161822, 161823, 161824, 161825 were selected from the ORFs having the nucleotide sequence represented by SEQ ID NO: 82397.

The probes having the nucleotide sequence represented by SEQ ID NOS: 161826, 161827, 161828, 161829, 161830, 161831, 161832, 161833, 161834, 161835, 161836 were selected from the ORFs having the nucleotide sequence represented by SEQ ID NO: 82398.

The probes having the nucleotide sequence represented by SEQ ID NOS: 161837, 161838, 161839, 161840, 161841, 161842, 161843, 161844, 161845, 161846, 161847 were selected from the ORFs having the nucleotide sequence represented by SEQ ID NO: 82399.

The probes having the nucleotide sequence represented by SEQ ID NOS: 161848, 161849, 161850, 161851, 161852, 161853, 161854, 161855, 161856, 161857, 161858 were selected from the ORFs having the nucleotide sequence represented by SEQ ID NO: 82400.

The probes having the nucleotide sequence represented by SEQ ID NOS: 161859, 161860, 161861, 161862, 161863, 161864, 161865, 161866, 161867, 161868, 161869 were selected from the ORFs having the nucleotide sequence represented by SEQ ID NO: 82401.

The probes having the nucleotide sequence represented by SEQ ID NOS: 161870, 161871, 161872, 161873, 161874, 161875, 161876, 161877, 161878, 161879, 161880 were selected from the ORFs having the nucleotide sequence represented by SEQ ID NO: 82402.

The probes having the nucleotide sequence represented by SEQ ID NOS: 161881, 161882, 161883, 161884, 161885, 161886, 161887, 161888, 161889, 161890, 161891 were selected from the ORFs having the nucleotide sequence represented by SEQ ID NO: 82403.

The probes having the nucleotide sequence represented by SEQ ID NOS: 161892, 161893, 161894, 161895, 161896, 161897, 161898, 161899, 161900, 161901, 161902 were selected from the ORFs having the nucleotide sequence represented by SEQ ID NO: 82404.

The probes having the nucleotide sequence represented by SEQ ID NOS: 161903, 161904, 161905, 161906, 161907, 161908, 161909, 161910, 161911, 161912, 161913 were selected from the ORFs having the nucleotide sequence represented by SEQ ID NO: 82405.

The probes having the nucleotide sequence represented by SEQ ID NOS: 161914, 161915, 161916, 161917, 161918, 161919, 161920, 161921, 161922, 161923, 161924 were selected from the ORFs having the nucleotide sequence represented by SEQ ID NO: 82406.

The probes having the nucleotide sequence represented by SEQ ID NOS:. 161925, 161926, 161927, 161928, 161929, 161930, 161931, 161932, 161933, 161934, 161935 were selected from the ORFs having the nucleotide sequence represented by SEQ ID NO: 82407.

The probes having the nucleotide sequence represented by SEQ ID NOS: 161936, 161937, 161938, 161939, 161940, 161941, 161942, 161943, 161944, 161945, 161946 were selected from the ORFs having the nucleotide sequence represented by SEQ ID NO: 82408.

The probes having the nucleotide sequence represented by SEQ ID NOS: 161947, 161948, 161949, 161950, 161951, 161952, 161953, 161954, 161955, 161956, 161957 were selected from the ORFs having the nucleotide sequence represented by SEQ ID NO: 82409.

The probes having the nucleotide sequence represented by SEQ ID NOS: 161958, 161959, 161960, 161961, 161962, 161963, 161964, 161965, 161966, 161967, 161968 were selected from the ORFs having the nucleotide sequence represented by SEQ ID NO: 82410.

The probes having the nucleotide sequence represented by SEQ ID NOS: 161969, 161970, 161971, 161972, 161973, 161974, 161975, 161976, 161977, 161978, 161979 were selected from the ORFs having the nucleotide sequence represented by SEQ ID NO: 82411.

The probes having the nucleotide sequence represented by SEQ ID NOS: 161980, 161981, 161982, 161983, 161984, 161985, 161986, 161987, 161988, 161989, 161990 were selected from the ORFs having the nucleotide sequence represented by SEQ ID NO: 82412.

The probes having the nucleotide sequence represented by SEQ ID NOS: 161991, 161992, 161993, 161994, 161995, 161996, 161997, 161998, 161999, 162000, 162001 were selected from the ORFs having the nucleotide sequence represented by SEQ ID NO: 82413.

The probes having the nucleotide sequence represented by SEQ ID NOS: 162002, 162003, 162004, 162005, 162006, 162007, 162008, 162009, 162010, 162011, 162012 were selected from the ORFs having the nucleotide sequence represented by SEQ ID NO: 82414.

The probes having the nucleotide sequence represented by SEQ ID NOS: 162013, 162014, 162015, 162016, 162017, 162018, 162019, 162020, 162021, 162022, 162023 were selected from the ORFs having the nucleotide sequence represented by SEQ ID NO: 82415.

The probes having the nucleotide sequence represented by SEQ ID NOS: 162024, 162025, 162026, 162027, 162028, 162029, 162030, 162031, 162032, 162033, 162034 were selected from the ORFs having the nucleotide sequence represented by SEQ ID NO: 82416.

The probes having the nucleotide sequence represented by SEQ ID NOS: 162035, 162036, 162037, 162038, 162039, 162040, 162041, 162042, 162043, 162044, 162045 were selected from the ORFs having the nucleotide sequence represented by SEQ ID NO: 82417.

The probes having the nucleotide sequence represented by SEQ ID NOS: 162046, 162047, 162048, 162049, 162050, 162051, 162052, 162053, 162054, 162055, 162056 were selected from the ORFs having the nucleotide sequence represented by SEQ ID NO: 82418.

The probes having the nucleotide sequence represented by SEQ ID NOS: 162057, 162058, 162059, 162060, 162061, 162062, 162063, 162064, 162065, 162066, 162067 were selected from the ORFs having the nucleotide sequence represented by SEQ ID NO: 82419.

The probes having the nucleotide sequence represented by SEQ ID NOS: 162068, 162069, 162070, 162071, 162072, 162073, 162074, 162075, 162076, 162077, 162078 were selected from the ORFs having the nucleotide sequence represented by SEQ ID NO: 82420.

The probes having the nucleotide sequence represented by SEQ ID NOS: 162079, 162080, 162081, 162082, 162083, 162084, 162085, 162086, 162087, 162088, 162089 were selected from the ORFs having the nucleotide sequence represented by SEQ ID NO: 82421.

The probes having the nucleotide sequence represented by SEQ ID NOS: 162090, 162091, 162092, 162093, 162094, 162095, 162096, 162097, 162098, 162099, 162100 were selected from the ORFs having the nucleotide sequence represented by SEQ ID NO: 82422.

The probes having the nucleotide sequence represented by SEQ ID NOS: 162101, 162102, 162103, 162104, 162105, 162106, 162107, 162108, 162109, 162110,: 162111 were selected from the ORFs having the nucleotide sequence represented by SEQ ID NO: 82423.

The probes having the nucleotide sequence represented by SEQ ID NOS: 162112, 162113, 162114, 162115, 162116, 162117, 162118, 162119, 162120, 162121, 162122 were selected from the ORFs having the nucleotide sequence represented by SEQ ID NO: 82424.

The probes having the nucleotide sequence represented by SEQ ID NOS: 162123, 162124, 162125, 162126, 162127, 162128, 162129, 162130, 162131, 162132, 162133 were selected from the ORFs having the nucleotide sequence represented by SEQ ID NO: 82425.

The probes having the nucleotide sequence represented by SEQ ID NOS: 162134, 162135, 162136, 162137, 162138, 162139, 162140, 162141, 162142, 162143, 162144 were selected from the ORFs having the nucleotide sequence represented by SEQ ID NO: 82426.

The probes having the nucleotide sequence represented by SEQ ID NOS: 162145, 162146, 162147, 162148, 162149, 162150, 162151, 162152, 162153, 162154, 162155 were selected from the ORFs having the nucleotide sequence represented by SEQ ID NO: 82427.

The probes having the nucleotide sequence represented by SEQ ID NOS: 162156, 162157, 162158, 162159, 162160, 162161, 162162, 162163, 162164, 162165, 162166 were selected from the ORFs having the nucleotide sequence represented by SEQ ID NO: 82428.

The probes having the nucleotide sequence represented by SEQ ID NOS: 162167, 162168, 162169, 162170, 162171, 162172, 162173, 162174, 162175, 162176, 162177 were selected from the ORFs having the nucleotide sequence represented by SEQ ID NO: 82429.

The probes having the nucleotide sequence represented by SEQ ID NOS: 162178, 162179, 162180, 162181, 162182, 162183, 162184, 162185, 162186, 162187, 162188 were selected from the ORFs having the nucleotide sequence represented by SEQ ID NO: 82430.

The probes having the nucleotide sequence represented by SEQ ID NOS: 162189, 162190, 162191, 162192, 162193, 162194, 162195, 162196, 162197, 162198, 162199 were selected from the ORFs having the nucleotide sequence represented by SEQ ID NO: 82431.

The probes having the nucleotide sequence represented by SEQ ID NOS: 162200, 162201, 162202, 162203, 162204, 162205, 162206, 162207, 162208, 162209, 162210 were selected from the ORFs having the nucleotide sequence represented by SEQ ID NO: 82432.

The probes having the nucleotide sequence represented by SEQ ID NOS: 162211, 162212, 162213, 162214, 162215, 162216, 162217, 162218, 162219, 162220, 162221 were selected from the ORFs having the nucleotide sequence represented by SEQ ID NO: 82433.

The probes having the nucleotide sequence represented by SEQ ID NOS: 162222, 162223, 162224, 162225, 162226, 162227, 162228, 162229, 162230, 162231, 162232 were selected from the ORFs having the nucleotide sequence represented by SEQ ID NO: 82434.

The probes having the nucleotide sequence represented by SEQ ID NOS: 162233, 162234, 162235, 162236, 162237, 162238, 162239, 162240, 162241, 162242, 162243 were selected from the ORFs having the nucleotide sequence represented by SEQ ID NO: 82435.

The probes having the nucleotide sequence represented by SEQ ID NOS: 162244, 162245, 162246, 162247, 162248, 162249, 162250, 162251, 162252, 162253, 162254 were selected from the ORFs having the nucleotide sequence represented by SEQ ID NO: 82436.

The probes having the nucleotide sequence represented by SEQ ID NOS: 162255, 162256, 162257, 162258, 162259, 162260, 162261, 162262, 162263, 162264, 162265 were selected from the ORFs having the nucleotide sequence represented by SEQ ID NO: 82437.

The probes having the nucleotide sequence represented by SEQ ID NOS: 162266, 162267, 162268, 162269, 162270, 162271, 162272, 162273, 162274, 162275, 162276 were selected from the ORFs having the nucleotide sequence represented by SEQ ID NO: 82438.

The probes having the nucleotide sequence represented by SEQ ID NOS: 162277, 162278, 162279, 162280, 162281, 162282, 162283, 162284, 162285, 162286, 162287 were selected from the ORFs having the nucleotide sequence represented by SEQ ID NO: 82439.

The probes having the nucleotide sequence represented by SEQ ID NOS: 162288, 162289, 162290, 162291, 162292, 162293, 162294, 162295, 162296, 162297, 162298 were selected from the ORFs having the nucleotide sequence represented by SEQ ID NO: 82440.

The probes having the nucleotide sequence represented by SEQ ID NOS: 162299, 162300, 162301, 162302, 162303, 162304, 162305, 162306, 162307, 162308, 162309 were selected from the ORFs having the nucleotide sequence represented by SEQ ID NO: 82441.

The probes having the nucleotide sequence represented by SEQ ID NOS: 162310, 162311, 162312, 162313, 162314, 162315, 162316, 162317, 162318, 162319, 162320 were selected from the ORFs having the nucleotide sequence represented by SEQ ID NO: 82442.

The probes having the nucleotide sequence represented by SEQ ID NOS: 162321, 162322, 162323, 162324, 162325, 162326, 162327, 162328, 162329, 162330, 162331 were selected from the ORFs having the nucleotide sequence represented by SEQ ID NO: 82443.

The probes having the nucleotide sequence represented by SEQ ID NOS: 162332, 162333, 162334, 162335, 162336, 162337, 162338, 162339, 162340, 162341, 162342, 162343, 162344, 162345, 162346, 162347, 162348, 162349, 162350, 162351, 162352, 162353 were selected from the ORFs having the nucleotide sequence represented by SEQ ID NO: 82444.

The probes having the nucleotide sequence represented by SEQ ID NOS: 162354, 162355, 162356, 162357, 162358, 162359, 162360, 162361, 162362, 162363, 162364 were selected from the ORFs having the nucleotide sequence represented by SEQ ID NO: 82445.

The probes having the nucleotide sequence represented by SEQ ID NOS: 162365, 162366, 162367, 162368, 162369, 162370, 162371, 162372, 162373, 162374, 162375 were selected from the ORFs having the nucleotide sequence represented by SEQ ID NO: 82446.

The probes having the nucleotide sequence represented by SEQ ID NOS: 162376, 162377, 162378, 162379, 162380, 162381, 162382, 162383, 162384, 162385, 162386 were selected from the ORFs having the nucleotide sequence represented by SEQ ID NO: 82447.

The probes having the nucleotide sequence represented by SEQ ID NOS: 162387, 162388, 162389, 162390, 162391, 162392, 162393, 162394, 162395, 162396, 162397 were selected from the ORFs having the nucleotide sequence represented by SEQ ID NO: 82448.

The probes having the nucleotide sequence represented by SEQ ID NOS: 162398, 162399, 162400, 162401, 162402, 162403, 162404, 162405, 162406, 162407, 162408 were selected from the ORFs having the nucleotide sequence represented by SEQ ID NO: 82449.

The probes having the nucleotide sequence represented by SEQ ID NOS: 162409, 162410, 162411, 162412, 162413, 162414, 162415, 162416, 162417, 162418, 162419 were selected from the ORFs having the nucleotide sequence represented by SEQ ID NO: 82450.

The probes having the nucleotide sequence represented by SEQ ID NOS: 162420, 162421, 162422, 162423, 162424, 162425, 162426, 162427, 162428, 162429, 162430 were selected from the ORFs having the nucleotide sequence represented by SEQ ID NO: 82451.

The probes having the nucleotide sequence represented by SEQ ID NOS: 162431, 162432, 162433, 162434, 162435, 162436, 162437, 162438, 162439, 162440, 162441 were selected from the ORFs having the nucleotide sequence represented by SEQ ID NO: 82452.

The probes having the nucleotide sequence represented by SEQ ID NOS: 162442, 162443, 162444, 162445, 162446, 162447, 162448, 162449, 162450, 162451, 162452 were selected from the ORFs having the nucleotide sequence represented by SEQ ID NO: 82453.

The probes having the nucleotide sequence represented by SEQ ID NOS: 162453, 162454, 162455, 162456, 162457, 162458, 162459, 162460, 162461, 162462, 162463 were selected from the ORFs having the nucleotide sequence represented by SEQ ID NO: 82454.

The probes having the nucleotide sequence represented by SEQ ID NOS: 162464, 162465, 162466, 162467, 162468, 162469, 162470, 162471, 162472, 162473, 162474 were selected from the ORFs having the nucleotide sequence represented by SEQ ID NO: 82455.

The probes having the nucleotide sequence represented by SEQ ID NOS: 162475, 162476, 162477, 162478, 162479, 162480, 162481, 162482, 162483, 162484, 162485 were selected from the ORFs having the nucleotide sequence represented by SEQ ID NO: 82456.

The probes having the nucleotide sequence represented by SEQ ID NOS: 162486, 162487, 162488, 162489, 162490, 162491, 162492, 162493, 162494, 162495, 162496 were selected from the ORFs having the nucleotide sequence represented by SEQ ID NO: 82457.

The probes having the nucleotide sequence represented by SEQ ID NOS: 162497, 162498, 162499, 162500, 162501, 162502, 162503, 162504, 162505, 162506, 162507 were selected from the ORFs having the nucleotide sequence represented by SEQ ID NO: 82458.

The probes having the nucleotide sequence represented by SEQ ID NOS: 162508, 162509, 162510, 162511, 162512, 162513, 162514, 162515 were selected from the ORFs having the nucleotide sequence represented by SEQ ID NO: 82459.

The probes having the nucleotide sequence represented by SEQ ID NOS: 162516, 162517, 162518, 162519, 162520, 162521, 162522, 162523, 162524, 162525, 162526 were selected from the ORFs having the nucleotide sequence represented by SEQ ID NO: 82460.

The probes having the nucleotide sequence represented by SEQ ID NOS: 162527, 162528, 162529, 162530, 162531, 162532, 162533, 162534, 162535, 162536, 162537 were selected from the ORFs having the nucleotide sequence represented by SEQ ID NO: 82461.

The probes having the nucleotide sequence represented by SEQ ID NOS: 162538, 162539, 162540, 162541, 162542, 162543, 162544, 162545, 162546, 162547, 162548 were selected from the ORFs having the nucleotide sequence represented by SEQ ID NO: 82462.

The probes having the nucleotide sequence represented by SEQ ID NOS: 162549, 162550, 162551, 162552, 162553, 162554, 162555, 162556, 162557, 162558, 162559 were selected from the ORFs having the nucleotide sequence represented by SEQ ID NO: 82463.

The probes having the nucleotide sequence represented by SEQ ID NOS: 162560, 162561, 162562, 162563, 162564, 162565, 162566, 162567, 162568, 162569, 162570 were selected from the ORFs having the nucleotide sequence represented by SEQ ID NO: 82464.

The probes having the nucleotide sequence represented by SEQ ID NOS: 162571, 162572, 162573, 162574, 162575, 162576, 162577, 162578, 162579, 162580, 162581 were selected from the ORFs having the nucleotide sequence represented by SEQ ID NO: 82465.

The probes having the nucleotide sequence represented by SEQ ID NOS: 162582, 162583, 162584, 162585, 162586, 162587, 162588, 162589, 162590, 162591, 162592 were selected from the ORFs having the nucleotide sequence represented by SEQ ID NO: 82466.

The probes having the nucleotide sequence represented by SEQ ID NOS: 162593, 162594, 162595, 162596, 162597, 162598, 162599, 162600, 162601, 162602, 162603 were selected from the ORFs having the nucleotide sequence represented by SEQ ID NO: 82467.

The probes having the nucleotide sequence represented by SEQ ID NOS: 162604, 162605, 162606, 162607, 162608, 162609, 162610, 162611, 162612, 162613, 162614 were selected from the ORFs having the nucleotide sequence represented by SEQ ID NO: 82468.

The probes having the nucleotide sequence represented by SEQ ID NOS: 162615, 162616, 162617, 162618, 162619, 162620, 162621, 162622, 162623, 162624, 162625 were selected from the ORFs having the nucleotide sequence represented by SEQ ID NO: 82469.

The probes having the nucleotide sequence represented by SEQ ID NOS: 162626, 162627, 162628, 162629, 162630, 162631, 162632, 162633, 162634, 162635, 162636 were selected from the ORFs having the nucleotide sequence represented by SEQ ID NO: 82470.

The probes having the nucleotide sequence represented by SEQ ID NOS: 162637, 162638, 162639, 162640, 162641, 162642, 162643, 162644, 162645, 162646, 162647 were selected from the ORFs having the nucleotide sequence represented by SEQ ID NO: 82471.

The probes having the nucleotide sequence represented by SEQ ID NOS: 162648, 162649, 162650, 162651, 162652, 162653, 162654, 162655, 162656, 162657, 162658 were selected from the ORFs having the nucleotide sequence represented by SEQ ID NO: 82472.

The probes having the nucleotide sequence represented by SEQ ID NOS: 162659, 162660, 162661, 162662, 162663, 162664, 162665, 162666, 162667, 162668, 162669 were selected from the ORFs having the nucleotide sequence represented by SEQ ID NO: 82473.

The probes having the nucleotide sequence represented by SEQ ID NOS: 162670, 162671, 162672, 162673, 162674, 162675, 162676, 162677, 162678, 162679, 162680 were selected from the ORFs having the nucleotide sequence represented by SEQ ID NO: 82474.

The probes having the nucleotide sequence represented by SEQ ID NOS: 162681, 162682, 162683, 162684, 162685, 162686, 162687, 162688, 162689, 162690, 162691 were selected from the ORFs having the nucleotide sequence represented by SEQ ID NO: 82475.

The probes having the nucleotide sequence represented by SEQ ID NOS: 162692, 162693, 162694, 162695, 162696, 162697, 162698, 162699, 162700, 162701, 162702 were selected from the ORFs having the nucleotide sequence represented by SEQ ID NO: 82476.

The probes having the nucleotide sequence represented by SEQ ID NOS: 162703, 162704, 162705, 162706, 162707, 162708, 162709, 162710, 162711, 162712, 162713 were selected from the ORFs having the nucleotide sequence represented by SEQ ID NO: 82477.

The probes having the nucleotide sequence represented by SEQ ID NOS: 162714, 162715, 162716, 162717, 162718, 162719, 162720, 162721, 162722, 162723, 162724 were selected from the ORFs having the nucleotide sequence represented by SEQ ID NO: 82478.

The probes having the nucleotide sequence represented by SEQ ID NOS: 162725, 162726, 162727, 162728, 162729, 162730, 162731, 162732, 162733, 162734, 162735 were selected from the ORFs having the nucleotide sequence represented by SEQ ID NO: 82479.

The probes having the nucleotide sequence represented by SEQ ID NOS: 162736, 162737, 162738, 162739, 162740, 162741, 162742, 162743, 162744, 162745, 162746 were selected from the ORFs having the nucleotide sequence represented by SEQ ID NO: 82480.

The probes having the nucleotide sequence represented by SEQ ID NOS: 162747, 162748, 162749, 162750, 162751, 162752, 162753, 162754, 162755, 162756, 162757 were selected from the ORFs having the nucleotide sequence represented by SEQ ID NO: 82481.

The probes having the nucleotide sequence represented by SEQ ID NOS: 162758, 162759, 162760, 162761, 162762, 162763, 162764, 162765, 162766, 162767, 162768 were selected from the ORFs having the nucleotide sequence represented by SEQ ID NO: 82482.

The probes having the nucleotide sequence represented by SEQ ID NOS: 162769, 162770, 162771, 162772, 162773, 162774, 162775, 162776, 162777, 162778, 162779 were selected from the ORFs having the nucleotide sequence represented by SEQ ID NO: 82483.

The probes having the nucleotide sequence represented by SEQ ID NOS: 162780, 162781, 162782, 162783, 162784, 162785, 162786, 162787, 162788, 162789, 162790 were selected from the ORFs having the nucleotide sequence represented by SEQ ID NO: 82484.

The probes having the nucleotide sequence represented by SEQ ID NOS: 162791, 162792, 162793, 162794, 162795, 162796, 162797, 162798, 162799, 162800, 162801 were selected from the ORFs having the nucleotide sequence represented by SEQ ID NO: 82485.

The probes having the nucleotide sequence represented by SEQ ID NOS: 162802, 162803, 162804, 162805, 162806, 162807, 162808, 162809, 162810, 162811, 162812 were selected from the ORFs having the nucleotide sequence represented by SEQ ID NO: 82486.

The probes having the nucleotide sequence represented by SEQ ID NOS: 162813, 162814, 162815, 162816, 162817, 162818, 162819, 162820, 162821, 162822, 162823 were selected from the ORFs having the nucleotide sequence represented by SEQ ID NO: 82487.

The probes having the nucleotide sequence represented by SEQ ID NOS: 162824, 162825, 162826, 162827, 162828, 162829, 162830, 162831, 162832, 162833, 162834 were selected from the ORFs having the nucleotide sequence represented by SEQ ID NO: 82488.

The probes having the nucleotide sequence represented by SEQ ID NOS: 162835, 162836, 162837, 162838, 162839, 162840, 162841, 162842, 162843, 162844, 162845 were selected from the ORFs having the nucleotide sequence represented by SEQ ID NO: 82489.

The probes having the nucleotide sequence represented by SEQ ID NOS: 162846, 162847, 162848, 162849, 162850, 162851, 162852, 162853, 162854, 162855, 162856 were selected from the ORFs having the nucleotide sequence represented by SEQ ID NO: 82490.

The probes having the nucleotide sequence represented by SEQ ID NOS: 162857, 162858, 162859, 162860, 162861, 162862, 162863, 162864, 162865, 162866, 162867 were selected from the ORFs having the nucleotide sequence represented by SEQ ID NO: 82491.

The probes having the nucleotide sequence represented by SEQ ID NOS: 162868, 162869, 162870, 162871, 162872, 162873, 162874, 162875, 162876, 162877, 162878 were selected from the ORFs having the nucleotide sequence represented by SEQ ID NO: 82492.

The probes having the nucleotide sequence represented by SEQ ID NOS: 162879, 162880, 162881, 162882, 162883, 162884, 162885, 162886, 162887, 162888, 162889 were selected from the ORFs having the nucleotide sequence represented by SEQ ID NO: 82493.

The probes having the nucleotide sequence represented by SEQ ID NOS: 162890, 162891, 162892, 162893, 162894, 162895, 162896, 162897, 162898, 162899, 162900 were selected from the ORFs having the nucleotide sequence represented by SEQ ID NO: 82494.

The probes having the nucleotide sequence represented by SEQ ID NOS: 162901, 162902, 162903, 162904, 162905, 162906, 162907, 162908, 162909, 162910, 162911 were selected from the ORFs having the nucleotide sequence represented by SEQ ID NO: 82495.

The probes having the nucleotide sequence represented by SEQ ID NOS: 162912, 162913, 162914, 162915, 162916, 162917, 162918, 162919, 162920, 162921, 162922 were selected from the ORFs having the nucleotide sequence represented by SEQ ID NO: 82496.

The probes having the nucleotide sequence represented by SEQ ID NOS: 162923, 162924, 162925, 162926, 162927, 162928, 162929, 162930, 162931, 162932, 162933 were selected from the ORFs having the nucleotide sequence represented by SEQ ID NO: 82497.

The probes having the nucleotide sequence represented by SEQ ID NOS: 162934, 162935, 162936, 162937, 162938, 162939, 162940, 162941, 162942, 162943, 162944 were selected from the ORFs having the nucleotide sequence represented by SEQ ID NO: 82498.

The probes having the nucleotide sequence represented by SEQ ID NOS: 162945, 162946, 162947, 162948, 162949, 162950, 162951, 162952, 162953, 162954, 162955 were selected from the ORFs having the nucleotide sequence represented by SEQ ID NO: 82499.

The probes having the nucleotide sequence represented by SEQ ID NOS: 162956, 162957, 162958, 162959, 162960, 162961, 162962, 162963, 162964, 162965, 162966 were selected from the ORFs having the nucleotide sequence represented by SEQ ID NO: 82500.

The probes having the nucleotide sequence represented by SEQ ID NOS: 162967, 162968, 162969, 162970, 162971, 162972, 162973, 162974, 162975, 162976, 162977 were selected from the ORFs having the nucleotide sequence represented by SEQ ID NO: 82501.

The probes having the nucleotide sequence represented by SEQ ID NOS: 162978, 162979, 162980, 162981, 162982, 162983, 162984, 162985, 162986, 162987, 162988 were selected from the ORFs having the nucleotide sequence represented by SEQ ID NO: 82502.

The probes having the nucleotide sequence represented by SEQ ID NOS: 162989, 162990, 162991, 162992, 162993, 162994, 162995, 162996, 162997, 162998, 162999 were selected from the ORFs having the nucleotide sequence represented by SEQ ID NO: 82503.

The probes having the nucleotide sequence represented by SEQ ID NOS: 163000, 163001, 163002, 163003, 163004, 163005, 163006, 163007, 163008, 163009, 163010 were selected from the ORFs having the nucleotide sequence represented by SEQ ID NO: 82504.

The probes having the nucleotide sequence represented by SEQ ID NOS: 163011, 163012, 163013, 163014, 163015, 163016, 163017, 163018, 163019, 163020, 163021 were selected from the ORFs having the nucleotide sequence represented by SEQ ID NO: 82505.

The probes having the nucleotide sequence represented by SEQ ID NOS: 163022, 163023, 163024, 163025, 163026, 163027, 163028, 163029, 163030, 163031, 163032 were selected from the ORFs having the nucleotide sequence represented by SEQ ID NO: 82506.

The probes having the nucleotide sequence represented by SEQ ID NOS: 163033, 163034, 163035, 163036, 163037, 163038, 163039, 163040, 163041, 163042, 163043 were selected from the ORFs having the nucleotide sequence represented by SEQ ID NO: 82507.

The probes having the nucleotide sequence represented by SEQ ID NOS: 163044, 163045, 163046, 163047, 163048, 163049, 163050, 163051, 163052, 163053, 163054 were selected from the ORFs having the nucleotide sequence represented by SEQ ID NO: 82508.

The probes having the nucleotide sequence represented by SEQ ID NOS: 163055, 163056, 163057, 163058, 163059, 163060, 163061, 163062, 163063, 163064, 163065 were selected from the ORFs having the nucleotide sequence represented by SEQ ID NO: 82509.

The probes having the nucleotide sequence represented by SEQ ID NOS: 163066, 163067, 163068, 163069, 163070, 163071, 163072, 163073, 163074, 163075, 163076 were selected from the ORFs having the nucleotide sequence represented by SEQ ID NO: 82510.

The probes having the nucleotide sequence represented by SEQ ID NOS: 163077, 163078, 163079, 163080, 163081, 163082, 163083, 163084, 163085, 163086, 163087 were selected from the ORFs having the nucleotide sequence represented by SEQ ID NO: 82511.

The probes having the nucleotide sequence represented by SEQ ID NOS: 163088, 163089, 163090, 163091, 163092, 163093, 163094, 163095, 163096, 163097, 163098 were selected from the ORFs having the nucleotide sequence represented by SEQ ID NO: 82512.

The probes having the nucleotide sequence represented by SEQ ID NOS: 163099, 163100, 163101, 163102, 163103, 163104, 163105, 163106, 163107, 163108, 163109 were selected from the ORFs having the nucleotide sequence represented by SEQ ID NO: 82513.

The probes having the nucleotide sequence represented by SEQ ID NOS: 163110, 163111, 163112, 163113, 163114, 163115, 163116, 163117, 163118, 163119, 163120 were selected from the ORFs having the nucleotide sequence represented by SEQ ID NO: 82514.

The probes having the nucleotide sequence represented by SEQ ID NOS: 163121, 163122, 163123, 163124, 163125, 163126, 163127, 163128, 163129, 163130, 163131 were selected from the ORFs having the nucleotide sequence represented by SEQ ID NO: 82515.

The probes having the nucleotide sequence represented by SEQ ID NOS: 163132, 163133, 163134, 163135, 163136, 163137, 163138, 163139, 163140, 163141, 163142 were selected from the ORFs having the nucleotide sequence represented by SEQ ID NO: 82516.

The probes having the nucleotide sequence represented by SEQ ID NOS: 163143, 163144, 163145, 163146, 163147, 163148, 163149, 163150, 163151, 163152, 163153 were selected from the ORFs having the nucleotide sequence represented by SEQ ID NO: 82517.

The probes having the nucleotide sequence represented by SEQ ID NOS: 163154, 163155, 163156, 163157, 163158, 163159, 163160, 163161, 163162, 163163, 163164 were selected from the ORFs having the nucleotide sequence represented by SEQ ID NO: 82518.

The probes having the nucleotide sequence represented by SEQ ID NOS: 163165, 163166, 163167, 163168, 163169, 163170, 163171, 163172, 163173, 163174, 163175 were selected from the ORFs having the nucleotide sequence represented by SEQ ID NO: 82519.

The probes having the nucleotide sequence represented by SEQ ID NOS: 163176, 163177, 163178, 163179, 163180, 163181, 163182, 163183, 163184, 163185, 163186 were selected from the ORFs having the nucleotide sequence represented by SEQ ID NO: 82520.

The probes having the nucleotide sequence represented by SEQ ID NOS: 163187, 163188, 163189, 163190, 163191, 163192, 163193, 163194, 163195, 163196, 163197 were selected from the ORFs having the nucleotide sequence represented by SEQ ID NO: 82521.

The probes having the nucleotide sequence represented by SEQ ID NOS: 163198, 163199, 163200, 163201, 163202, 163203, 163204, 163205, 163206, 163207, 163208 were selected from the ORFs having the nucleotide sequence represented by SEQ ID NO: 82522.

The probes having the nucleotide sequence represented by SEQ ID NOS: 163209, 163210, 163211, 163212, 163213, 163214, 163215, 163216, 163217, 163218, 163219 were selected from the ORFs having the nucleotide sequence represented by SEQ ID NO: 82523.

The probes having the nucleotide sequence represented by SEQ ID NOS: 163220, 163221, 163222, 163223, 163224, 163225, 163226, 163227, 163228, 163229, 163230 were selected from the ORFs having the nucleotide sequence represented by SEQ ID NO: 82524.

The probes having the nucleotide sequence represented by SEQ ID NOS: 163231, 163232, 163233, 163234, 163235, 163236, 163237, 163238, 163239, 163240, 163241 were selected from the ORFs having the nucleotide sequence represented by SEQ ID NO: 82525.

The probes having the nucleotide sequence represented by SEQ ID NOS: 163242, 163243, 163244, 163245, 163246, 163247, 163248, 163249, 163250, 163251, 163252 were selected from the ORFs having the nucleotide sequence represented by SEQ ID NO: 82526.

The probes having the nucleotide sequence represented by SEQ ID NOS: 163253, 163254, 163255, 163256, 163257, 163258, 163259, 163260, 163261, 163262, 163263 were selected from the ORFs having the nucleotide sequence represented by SEQ ID NO: 82527.

The probes having the nucleotide sequence represented by SEQ ID NOS: 163264, 163265, 163266, 163267, 163268, 163269, 163270, 163271, 163272, 163273, 163274 were selected from the ORFs having the nucleotide sequence represented by SEQ ID NO: 82528.

The probes having the nucleotide sequence represented by SEQ ID NOS: 163275, 163276, 163277, 163278, 163279, 163280, 163281, 163282, 163283, 163284, 163285 were selected from the ORFs having the nucleotide sequence represented by SEQ ID NO: 82529.

The probes having the nucleotide sequence represented by SEQ ID NOS: 163286, 163287, 163288, 163289, 163290, 163291, 163292, 163293, 163294, 163295, 163296 were selected from the ORFs having the nucleotide sequence represented by SEQ ID NO: 82530.

The probes having the nucleotide sequence represented by SEQ ID NOS: 163297, 163298, 163299, 163300, 163301, 163302, 163303, 163304, 163305, 163306, 163307 were selected from the ORFs having the nucleotide sequence represented by SEQ ID NO: 82531.

The probes having the nucleotide sequence represented by SEQ ID NOS: 163308, 163309, 163310, 163311, 163312, 163313, 163314, 163315, 163316, 163317, 163318 were selected from the ORFs having the nucleotide sequence represented by SEQ ID NO: 82532.

The probes having the nucleotide sequence represented by SEQ ID NOS: 163319, 163320, 163321, 163322, 163323, 163324, 163325, 163326, 163327, 163328, 163329 were selected from the ORFs having the nucleotide sequence represented by SEQ ID NO: 82533.

The probes having the nucleotide sequence represented by SEQ ID NOS: 163330, 163331, 163332, 163333, 163334, 163335, 163336, 163337, 163338, 163339, 163340 were selected from the ORFs having the nucleotide sequence represented by SEQ ID NO: 82534.

The probes having the nucleotide sequence represented by SEQ ID NOS: 163341, 163342, 163343, 163344, 163345, 163346, 163347, 163348, 163349, 163350, 163351 were selected from the ORFs having the nucleotide sequence represented by SEQ ID NO: 82535.

The probes having the nucleotide sequence represented by SEQ ID NOS: 163352, 163353, 163354, 163355, 163356, 163357, 163358, 163359, 163360, 163361, 163362 were selected from the ORFs having the nucleotide sequence represented by SEQ ID NO: 82536.

The probes having the nucleotide sequence represented by SEQ ID NOS: 163363, 163364, 163365, 163366, 163367, 163368, 163369, 163370, 163371, 163372, 163373 were selected from the ORFs having the nucleotide sequence represented by SEQ ID NO: 82537.

The probes having the nucleotide sequence represented by SEQ ID NOS: 163374, 163375, 163376, 163377, 163378, 163379, 163380, 163381, 163382, 163383, 163384 were selected from the ORFs having the nucleotide sequence represented by SEQ ID NO: 82538.

The probes having the nucleotide sequence represented by SEQ ID NOS: 163385, 163386, 163387, 163388, 163389, 163390, 163391, 163392, 163393, 163394, 163395 were selected from the ORFs having the nucleotide sequence represented by SEQ ID NO: 82539.

The probes having the nucleotide sequence represented by SEQ ID NOS: 163396, 163397, 163398, 163399, 163400, 163401, 163402, 163403, 163404, 163405, 163406 were selected from the ORFs having the nucleotide sequence represented by SEQ ID NO: 82540.

The probes having the nucleotide sequence represented by SEQ ID NOS: 163407, 163408, 163409, 163410, 163411, 163412, 163413, 163414, 163415, 163416, 163417 were selected from the ORFs having the nucleotide sequence represented by SEQ ID NO: 82541.

The probes having the nucleotide sequence represented by SEQ ID NOS: 163418, 163419, 163420, 163421, 163422, 163423, 163424, 163425, 163426, 163427, 163428 were selected from the ORFs having the nucleotide sequence represented by SEQ ID NO: 82542.

The probes having the nucleotide sequence represented by SEQ ID NOS: 163429, 163430, 163431, 163432, 163433, 163434, 163435, 163436, 163437, 163438, 163439 were selected from the ORFs having the nucleotide sequence represented by SEQ ID NO: 82543.

The probes having the nucleotide sequence represented by SEQ ID NOS: 163440, 163441, 163442, 163443, 163444, 163445, 163446, 163447, 163448, 163449, 163450 were selected from the ORFs having the nucleotide sequence represented by SEQ ID NO: 82544.

The probes having the nucleotide sequence represented by SEQ ID NOS: 163451, 163452, 163453, 163454, 163455, 163456, 163457, 163458, 163459, 163460, 163461 were selected from the ORFs having the nucleotide sequence represented by SEQ ID NO: 82545.

The probes having the nucleotide sequence represented by SEQ ID NOS: 163462, 163463, 163464, 163465, 163466, 163467, 163468, 163469, 163470, 163471, 163472 were selected from the ORFs having the nucleotide sequence represented by SEQ ID NO: 82546.

The probes having the nucleotide sequence represented by SEQ ID NOS: 163473, 163474, 163475, 163476, 163477, 163478, 163479, 163480, 163481, 163482, 163483 were selected from the ORFs having the nucleotide sequence represented by SEQ ID NO: 82547.

The probes having the nucleotide sequence represented by SEQ ID NOS: 163484, 163485, 163486, 163487, 163488, 163489, 163490, 163491, 163492, 163493, 163494 were selected from the ORFs having the nucleotide sequence represented by SEQ ID NO: 82548.

The probes having the nucleotide sequence represented by SEQ ID NOS: 163495, 163496, 163497, 163498, 163499, 163500, 163501, 163502, 163503, 163504, 163505 were selected from the ORFs having the nucleotide sequence represented by SEQ ID NO: 82549.

The probes having the nucleotide sequence represented by SEQ ID NOS: 163506, 163507, 163508, 163509, 163510, 163511, 163512, 163513, 163514, 163515, 163516 were selected from the ORFs having the nucleotide sequence represented by SEQ ID NO: 82550.

The probes having the nucleotide sequence represented by SEQ ID NOS: 163517, 163518, 163519, 163520, 163521, 163522, 163523, 163524, 163525, 163526, 163527 were selected from the ORFs having the nucleotide sequence represented by SEQ ID NO: 82551.

The probes having the nucleotide sequence represented by SEQ ID NOS: 163528, 163529, 163530, 163531, 163532, 163533, 163534, 163535, 163536, 163537, 163538 were selected from the ORFs having the nucleotide sequence represented by SEQ ID NO: 82552.

The probes having the nucleotide sequence represented by SEQ ID NOS: 163539, 163540, 163541, 163542, 163543, 163544, 163545, 163546, 163547, 163548, 163549, 163550, 163551, 163552, 163553, 163554, 163555, 163556, 163557, 163558, 163559, 163560 were selected from the ORFs having the nucleotide sequence represented by SEQ ID NO: 82553.

The probes having the nucleotide sequence represented by SEQ ID NOS: 163561, 163562, 163563, 163564, 163565, 163566, 163567, 163568, 163569, 163570, 163571 were selected from the ORFs having the nucleotide sequence represented by SEQ ID NO: 82554.

The probes having the nucleotide sequence represented by SEQ ID NOS: 163572, 163573, 163574, 163575, 163576, 163577, 163578, 163579, 163580, 163581, 163582 were selected from the ORFs having the nucleotide sequence represented by SEQ ID NO: 82555.

The probes having the nucleotide sequence represented by SEQ ID NOS: 163583, 163584, 163585, 163586, 163587, 163588, 163589, 163590, 163591, 163592, 163593 were selected from the ORFs having the nucleotide sequence represented by SEQ ID NO: 82556.

The probes having the nucleotide sequence represented by SEQ ID NOS: 163594, 163595, 163596, 163597, 163598, 163599, 163600, 163601, 163602, 163603, 163604 were selected from the ORFs having the nucleotide sequence represented by SEQ ID NO: 82557.

The probes having the nucleotide sequence represented by SEQ ID NOS: 163605, 163606, 163607, 163608, 163609, 163610, 163611, 163612, 163613, 163614, 163615 were selected from the ORFs having the nucleotide sequence represented by SEQ ID NO: 82558.

The probes having the nucleotide sequence represented by SEQ ID NOS: 163616, 163617, 163618, 163619, 163620, 163621, 163622, 163623, 163624, 163625, 163626 were selected from the ORFs having the nucleotide sequence represented by SEQ ID NO: 82559.

The probes having the nucleotide sequence represented by SEQ ID NOS: 163627, 163628, 163629, 163630, 163631, 163632, 163633, 163634, 163635, 163636, 163637 were selected from the ORFs having the nucleotide sequence represented by SEQ ID NO: 82560.

The probes having the nucleotide sequence represented by SEQ ID NOS: 163638, 163639, 163640, 163641, 163642, 163643, 163644, 163645, 163646, 163647, 163648 were selected from the ORFs having the nucleotide sequence represented by SEQ ID NO: 82561.

The probes having the nucleotide sequence represented by SEQ ID NOS: 163649, 163650, 163651, 163652, 163653, 163654, 163655, 163656, 163657, 163658, 163659 were selected from the ORFs having the nucleotide sequence represented by SEQ ID NO: 82562.

The probes having the nucleotide sequence represented by SEQ ID NOS: 163660, 163661, 163662, 163663, 163664, 163665, 163666, 163667, 163668, 163669, 163670 were selected from the ORFs having the nucleotide sequence represented by SEQ ID NO: 82563.

The probes having the nucleotide sequence represented by SEQ ID NOS: 163671, 163672, 163673, 163674, 163675, 163676, 163677, 163678, 163679, 163680, 163681 were selected from the ORFs having the nucleotide sequence represented by SEQ ID NO: 82564.

The probes having the nucleotide sequence represented by SEQ ID NOS: 163682, 163683, 163684, 163685, 163686, 163687, 163688, 163689, 163690 were selected from the ORFs having the nucleotide sequence represented by SEQ ID NO: 82565.

The probes having the nucleotide sequence represented by SEQ ID NOS: 163691, 163692, 163693, 163694, 163695, 163696, 163697, 163698, 163699, 163700, 163701 were selected from the ORFs having the nucleotide sequence represented by SEQ ID NO: 82566.

The probes having the nucleotide sequence represented by SEQ ID NOS: 163702, 163703, 163704, 163705, 163706, 163707, 163708, 163709, 163710, 163711, 163712 were selected from the ORFs having the nucleotide sequence represented by SEQ ID NO: 82567.

The probes having the nucleotide sequence represented by SEQ ID NOS: 163713, 163714, 163715, 163716, 163717, 163718, 163719, 163720, 163721, 163722, 163723 were selected from the ORFs having the nucleotide sequence represented by SEQ ID NO: 82568.

The probes having the nucleotide sequence represented by SEQ ID NOS: 163724, 163725, 163726, 163727, 163728, 163729, 163730, 163731, 163732, 163733, 163734 were selected from the ORFs having the nucleotide sequence represented by SEQ ID NO: 82569.

The probes having the nucleotide sequence represented by SEQ ID NOS: 163735, 163736, 163737, 163738, 163739, 163740, 163741, 163742, 163743, 163744, 163745 were selected from the ORFs having the nucleotide sequence represented by SEQ ID NO: 82570.

The probes having the nucleotide sequence represented by SEQ ID NOS: 163746, 163747, 163748, 163749, 163750, 163751, 163752, 163753, 163754, 163755, 163756 were selected from the ORFs having the nucleotide sequence represented by SEQ ID NO: 82571.

The probes having the nucleotide sequence represented by SEQ ID NOS: 163757, 163758, 163759, 163760, 163761, 163762, 163763, 163764, 163765, 163766, 163767 were selected from the ORFs having the nucleotide sequence represented by SEQ ID NO: 82572.

The probes having the nucleotide sequence represented by SEQ ID NOS: 163768, 163769, 163770, 163771, 163772, 163773, 163774, 163775, 163776, 163777, 163778 were selected from the ORFs having the nucleotide sequence represented by SEQ ID NO: 82573.

The probes having the nucleotide sequence represented by SEQ ID NOS: 163779, 163780, 163781, 163782, 163783, 163784, 163785, 163786, 163787, 163788, 163789 were selected from the ORFs having the nucleotide sequence represented by SEQ ID NO: 82574.

The probes having the nucleotide sequence represented by SEQ ID NOS: 163790, 163791, 163792, 163793, 163794, 163795, 163796, 163797, 163798, 163799, 163800 were selected from the ORFs having the nucleotide sequence represented by SEQ ID NO: 82575.

The probes having the nucleotide sequence represented by SEQ ID NOS: 163801, 163802, 163803, 163804, 163805, 163806, 163807, 163808, 163809, 163810, 163811 were selected from the ORFs having the nucleotide sequence represented by SEQ ID NO: 82576.

The probes having the nucleotide sequence represented by SEQ ID NOS: 163812, 163813, 163814, 163815, 163816, 163817, 163818, 163819, 163820, 163821, 163822 were selected from the ORFs having the nucleotide sequence represented by SEQ ID NO: 82577.

The probes having the nucleotide sequence represented by SEQ ID NOS: 163823, 163824, 163825, 163826, 163827, 163828, 163829, 163830, 163831, 163832, 163833 were selected from the ORFs having the nucleotide sequence represented by SEQ ID NO: 82578.

The probes having the nucleotide sequence represented by SEQ ID NOS: 163834, 163835, 163836, 163837, 163838, 163839, 163840, 163841, 163842, 163843, 163844 were selected from the ORFs having the nucleotide sequence represented by SEQ ID NO: 82579.

The probes having the nucleotide sequence represented by SEQ ID NOS: 163845, 163846, 163847, 163848, 163849, 163850, 163851, 163852, 163853, 163854, 163855 were selected from the ORFs having the nucleotide sequence represented by SEQ ID NO: 82580.

The probes having the nucleotide sequence represented by SEQ ID NOS: 163856, 163857, 163858, 163859, 163860, 163861, 163862, 163863, 163864, 163865, 163866 were selected from the ORFs having the nucleotide sequence represented by SEQ ID NO: 82581.

The probes having the nucleotide sequence represented by SEQ ID NOS: 163867, 163868, 163869, 163870, 163871, 163872, 163873, 163874, 163875, 163876, 163877 were selected from the ORFs having the nucleotide sequence represented by SEQ ID NO: 82582.

The probes having the nucleotide sequence represented by SEQ ID NOS: 163878, 163879, 163880, 163881, 163882, 163883, 163884, 163885, 163886, 163887, 163888 were selected from the ORFs having the nucleotide sequence represented by SEQ ID NO: 82583.

The probes having the nucleotide sequence represented by SEQ ID NOS: 163889, 163890, 163891, 163892, 163893, 163894, 163895, 163896, 163897, 163898, 163899 were selected from the ORFs having the nucleotide sequence represented by SEQ ID NO: 82584.

The probes having the nucleotide sequence represented by SEQ ID NOS: 163900, 163901, 163902, 163903, 163904, 163905, 163906, 163907, 163908, 163909, 163910 were selected from the ORFs having the nucleotide sequence represented by SEQ ID NO: 82585.

The probes having the nucleotide sequence represented by SEQ ID NOS: 163911, 163912, 163913, 163914, 163915, 163916, 163917, 163918, 163919, 163920, 163921 were selected from the ORFs having the nucleotide sequence represented by SEQ ID NO: 82586.

The probes having the nucleotide sequence represented by SEQ ID NOS: 163922, 163923, 163924, 163925, 163926, 163927, 163928, 163929, 163930, 163931, 163932 were selected from the ORFs having the nucleotide sequence represented by SEQ ID NO: 82587.

The probes having the nucleotide sequence represented by SEQ ID NOS: 163933, 163934, 163935, 163936, 163937, 163938, 163939, 163940, 163941, 163942, 163943 were selected from the ORFs having the nucleotide sequence represented by SEQ ID NO: 82588.

The probes having the nucleotide sequence represented by SEQ ID NOS: 163944, 163945, 163946, 163947, 163948, 163949, 163950, 163951, 163952, 163953, 163954 were selected from the ORFs having the nucleotide sequence represented by SEQ ID NO: 82589.

The probes having the nucleotide sequence represented by SEQ ID NOS: 163955, 163956, 163957, 163958, 163959, 163960, 163961, 163962, 163963, 163964, 163965 were selected from the ORFs having the nucleotide sequence represented by SEQ ID NO: 82590.

The probes having the nucleotide sequence represented by SEQ ID NOS: 163966, 163967, 163968, 163969, 163970, 163971, 163972, 163973, 163974, 163975, 163976, 163977, 163978, 163979, 163980, 163981, 163982, 163983, 163984, 163985, 163986, 163987 were selected from the ORFs having the nucleotide sequence represented by SEQ ID NO: 82591.

The probes having the nucleotide sequence represented by SEQ ID NOS: 163988, 163989, 163990, 163991, 163992, 163993, 163994, 163995, 163996, 163997, 163998 were selected from the ORFs having the nucleotide sequence represented by SEQ ID NO: 82592.

The probes having the nucleotide sequence represented by SEQ ID NOS: 163999, 164000, 164001, 164002, 164003, 164004, 164005, 164006, 164007, 164008, 164009 were selected from the ORFs having the nucleotide sequence represented by SEQ ID NO: 82593.

The probes having the nucleotide sequence represented by SEQ ID NOS: 164010, 164011, 164012, 164013, 164014, 164015, 164016, 164017, 164018, 164019, 164020 were selected from the ORFs having the nucleotide sequence represented by SEQ ID NO: 82594.

The probes having the nucleotide sequence represented by SEQ ID NOS: 164021, 164022, 164023, 164024, 164025, 164026, 164027, 164028, 164029, 164030, 164031 were selected from the ORFs having the nucleotide sequence represented by SEQ ID NO: 82595.

The probes having the nucleotide sequence represented by SEQ ID NOS: 164032, 164033, 164034, 164035, 164036, 164037, 164038, 164039, 164040, 164041, 164042 were selected from the ORFs having the nucleotide sequence represented by SEQ ID NO: 82596.

The probes having the nucleotide sequence represented by SEQ ID NOS: 164043, 164044, 164045, 164046, 164047, 164048, 164049, 164050, 164051, 164052, 164053 were selected from the ORFs having the nucleotide sequence represented by SEQ ID NO: 82597.

The probes having the nucleotide sequence represented by SEQ ID NOS: 164054, 164055, 164056, 164057, 164058, 164059, 164060, 164061, 164062, 164063, 164064 were selected from the ORFs having the nucleotide sequence represented by SEQ ID NO: 82598.

The probes having the nucleotide sequence represented by SEQ ID NOS: 164065, 164066, 164067, 164068, 164069, 164070, 164071, 164072, 164073, 164074, 164075 were selected from the ORFs having the nucleotide sequence represented by SEQ ID NO: 82599.

The probes having the nucleotide sequence represented by SEQ ID NOS: 164076, 164077, 164078, 164079, 164080, 164081, 164082, 164083, 164084, 164085, 164086 were selected from the ORFs having the nucleotide sequence represented by SEQ ID NO: 82600.

The probes having the nucleotide sequence represented by SEQ ID NOS: 164087, 164088, 164089, 164090, 164091, 164092, 164093, 164094, 164095, 164096, 164097 were selected from the ORFs having the nucleotide sequence represented by SEQ ID NO: 82601.

The probes having the nucleotide sequence represented by SEQ ID NOS: 164098, 164099, 164100, 164101, 164102, 164103, 164104, 164105, 164106, 164107, 164108 were selected from the ORFs having the nucleotide sequence represented by SEQ ID NO: 82602.

The probes having the nucleotide sequence represented by SEQ ID NOS: 164109, 164110, 164111, 164112, 164113, 164114, 164115, 164116, 164117, 164118, 164119 were selected from the ORFs having the nucleotide sequence represented by SEQ ID NO: 82603.

The probes having the nucleotide sequence represented by SEQ ID NOS: 164120, 164121, 164122, 164123, 164124, 164125, 164126, 164127, 164128, 164129, 164130 were selected from the ORFs having the nucleotide sequence represented by SEQ ID NO: 82604.

The probes having the nucleotide sequence represented by SEQ ID NOS: 164131, 164132, 164133, 164134, 164135, 164136, 164137, 164138, 164139, 164140, 164141 were selected from the ORFs having the nucleotide sequence represented by SEQ ID NO: 82605.

The probes having the nucleotide sequence represented by SEQ ID NOS: 164142, 164143, 164144, 164145, 164146, 164147, 164148, 164149, 164150, 164151, 164152 were selected from the ORFs having the nucleotide sequence represented by SEQ ID NO: 82606.

The probes having the nucleotide sequence represented by SEQ ID NOS: 164153, 164154, 164155, 164156, 164157, 164158, 164159, 164160, 164161, 164162, 164163 were selected from the ORFs having the nucleotide sequence represented by SEQ ID NO: 82607.

The probes having the nucleotide sequence represented by SEQ ID NOS: 164164, 164165, 164166, 164167, 164168, 164169, 164170, 164171, 164172, 164173, 164174 were selected from the ORFs having the nucleotide sequence represented by SEQ ID NO: 82608.

The probes having the nucleotide sequence represented by SEQ ID NOS: 164175, 164176, 164177, 164178, 164179, 164180, 164181, 164182, 164183, 164184, 164185 were selected from the ORFs having the nucleotide sequence represented by SEQ ID NO: 82609.

The probes having the nucleotide sequence represented by SEQ ID NOS: 164186, 164187, 164188, 164189, 164190, 164191, 164192, 164193, 164194, 164195, 164196 were selected from the ORFs having the nucleotide sequence represented by SEQ ID NO: 82610.

The probes having the nucleotide sequence represented by SEQ ID NOS: 164197, 164198, 164199, 164200, 164201, 164202, 164203, 164204, 164205, 164206, 164207 were selected from the ORFs having the nucleotide sequence represented by SEQ ID NO: 82611.

The probes having the nucleotide sequence represented by SEQ ID NOS: 164208, 164209, 164210, 164211, 164212, 164213, 164214, 164215, 164216, 164217, 164218 were selected from the ORFs having the nucleotide sequence represented by SEQ ID NO: 82612.

The probes having the nucleotide sequence represented by SEQ ID NOS: 164219, 164220, 164221, 164222, 164223, 164224, 164225, 164226, 164227, 164228, 164229 were selected from the ORFs having the nucleotide sequence represented by SEQ ID NO: 82613.

The probes having the nucleotide sequence represented by SEQ ID NOS: 164230, 164231, 164232, 164233, 164234, 164235, 164236, 164237, 164238, 164239, 164240 were selected from the ORFs having the nucleotide sequence represented by SEQ ID NO: 82614.

The probes having the nucleotide sequence represented by SEQ ID NOS: 164241, 164242, 164243, 164244, 164245, 164246, 164247, 164248, 164249, 164250, 164251 were selected from the ORFs having the nucleotide sequence represented by SEQ ID NO: 82615.

The probes having the nucleotide sequence represented by SEQ ID NOS: 164252, 164253, 164254, 164255, 164256, 164257, 164258, 164259, 164260, 164261, 164262 were selected from the ORFs having the nucleotide sequence represented by SEQ ID NO: 82616.

The probes having the nucleotide sequence represented by SEQ ID NOS: 164263, 164264, 164265, 164266, 164267, 164268, 164269, 164270, 164271, 164272, 164273 were selected from the ORFs having the nucleotide sequence represented by SEQ ID NO: 82617.

The probes having the nucleotide sequence represented by SEQ ID NOS: 164274, 164275, 164276, 164277, 164278, 164279, 164280, 164281, 164282, 164283, 164284 were selected from the ORFs having the nucleotide sequence represented by SEQ ID NO: 82618.

The probes having the nucleotide sequence represented by SEQ ID NOS: 164285, 164286, 164287, 164288, 164289, 164290, 164291, 164292, 164293, 164294, 164295 were selected from the ORFs having the nucleotide sequence represented by SEQ ID NO: 82619.

The probes having the nucleotide sequence represented by SEQ ID NOS: 164296, 164297, 164298, 164299, 164300, 164301, 164302, 164303, 164304, 164305, 164306 were selected from the ORFs having the nucleotide sequence represented by SEQ ID NO: 82620.

The probes having the nucleotide sequence represented by SEQ ID NOS: 164307, 164308, 164309, 164310, 164311, 164312, 164313, 164314, 164315, 164316, 164317 were selected from the ORFs having the nucleotide sequence represented by SEQ ID NO: 82621.

The probes having the nucleotide sequence represented by SEQ ID NOS: 164318, 164319, 164320, 164321, 164322, 164323, 164324, 164325, 164326, 164327, 164328 were selected from the ORFs having the nucleotide sequence represented by SEQ ID NO: 82622.

The probes having the nucleotide sequence represented by SEQ ID NOS: 164329, 164330, 164331, 164332, 164333, 164334, 164335, 164336, 164337, 164338, 164339 were selected from the ORFs having the nucleotide sequence represented by SEQ ID NO: 82623.

The probes having the nucleotide sequence represented by SEQ ID NOS: 164340, 164341, 164342, 164343, 164344, 164345, 164346, 164347, 164348, 164349, 164350 were selected from the ORFs having the nucleotide sequence represented by SEQ ID NO: 82624.

The probes having the nucleotide sequence represented by SEQ ID NOS: 164351, 164352, 164353, 164354, 164355, 164356, 164357, 164358, 164359, 164360, 164361 were selected from the ORFs having the nucleotide sequence represented by SEQ ID NO: 82625.

The probes having the nucleotide sequence represented by SEQ ID NOS: 164362, 164363, 164364, 164365, 164366, 164367, 164368, 164369, 164370, 164371, 164372 were selected from the ORFs having the nucleotide sequence represented by SEQ ID NO: 82626.

The probes having the nucleotide sequence represented by SEQ ID NOS: 164373, 164374, 164375, 164376, 164377, 164378, 164379, 164380, 164381, 164382, 164383 were selected from the ORFs having the nucleotide sequence represented by SEQ ID NO: 82627.

The probes having the nucleotide sequence represented by SEQ ID NOS: 164384, 164385, 164386, 164387, 164388, 164389, 164390, 164391, 164392, 164393, 164394 were selected from the ORFs having the nucleotide sequence represented by SEQ ID NO: 82628.

The probes having the nucleotide sequence represented by SEQ ID NOS: 164395, 164396, 164397, 164398, 164399, 164400, 164401, 164402, 164403, 164404, 164405 were selected from the ORFs having the nucleotide sequence represented by SEQ ID NO: 82629.

The probes having the nucleotide sequence represented by SEQ ID NOS: 164406, 164407, 164408, 164409, 164410, 164411, 164412, 164413, 164414, 164415, 164416 were selected from the ORFs having the nucleotide sequence represented by SEQ ID NO: 82630.

The probes having the nucleotide sequence represented by SEQ ID NOS: 164417, 164418, 164419, 164420, 164421, 164422, 164423, 164424, 164425, 164426, 164427 were selected from the ORFs having the nucleotide sequence represented by SEQ ID NO: 82631.

The probes having the nucleotide sequence represented by SEQ ID NOS: 164428, 164429, 164430, 164431, 164432, 164433, 164434, 164435, 164436, 164437, 164438 were selected from the ORFs having the nucleotide sequence represented by SEQ ID NO: 82632.

The probes having the nucleotide sequence represented by SEQ ID NOS: 164439, 164440, 164441, 164442, 164443, 164444, 164445, 164446, 164447, 164448, 164449 were selected from the ORFs having the nucleotide sequence represented by SEQ ID NO: 82633.

The probes having the nucleotide sequence represented by SEQ ID NOS: 164450, 164451, 164452, 164453, 164454, 164455, 164456, 164457, 164458, 164459, 164460 were selected from the ORFs having the nucleotide sequence represented by SEQ ID NO: 82634.

The probes having the nucleotide sequence represented by SEQ ID NOS: 164461, 164462, 164463, 164464, 164465, 164466, 164467, 164468, 164469, 164470, 164471 were selected from the ORFs having the nucleotide sequence represented by SEQ ID NO: 82635.

The probes having the nucleotide sequence represented by SEQ ID NOS: 164472, 164473, 164474, 164475, 164476, 164477, 164478, 164479, 164480, 164481, 164482 were selected from the ORFs having the nucleotide sequence represented by SEQ ID NO: 82636.

The probes having the nucleotide sequence represented by SEQ ID NOS: 164483, 164484, 164485, 164486, 164487, 164488, 164489, 164490, 164491, 164492, 164493 were selected from the ORFs having the nucleotide sequence represented by SEQ ID NO: 82637.

The probes having the nucleotide sequence represented by SEQ ID NOS: 164494, 164495, 164496, 164497, 164498, 164499, 164500, 164501, 164502, 164503, 164504 were selected from the ORFs having the nucleotide sequence represented by SEQ ID NO: 82638.

The probes having the nucleotide sequence represented by SEQ ID NOS: 164505, 164506, 164507, 164508, 164509, 164510, 164511, 164512, 164513, 164514, 164515 were selected from the ORFs having the nucleotide sequence represented by SEQ ID NO: 82639.

The probes having the nucleotide sequence represented by SEQ ID NOS: 164516, 164517, 164518, 164519, 164520, 164521, 164522, 164523, 164524, 164525, 164526 were selected from the ORFs having the nucleotide sequence represented by SEQ ID NO: 82640.

The probes having the nucleotide sequence represented by SEQ ID NOS: 164527, 164528, 164529, 164530, 164531, 164532, 164533, 164534, 164535, 164536, 164537 were selected from the ORFs having the nucleotide sequence represented by SEQ ID NO: 82641.

The probes having the nucleotide sequence represented by SEQ ID NOS: 164538, 164539, 164540, 164541, 164542, 164543, 164544, 164545, 164546, 164547, 164548 were selected from the ORFs having the nucleotide sequence represented by SEQ ID NO: 82642.

The probes having the nucleotide sequence represented by SEQ ID NOS: 164549, 164550, 164551, 164552, 164553, 164554, 164555, 164556, 164557, 164558, 164559 were selected from the ORFs having the nucleotide sequence represented by SEQ ID NO: 82643.

The probes having the nucleotide sequence represented by SEQ ID NOS: 164560, 164561, 164562, 164563, 164564, 164565, 164566, 164567, 164568, 164569, 164570 were selected from the ORFs having the nucleotide sequence represented by SEQ ID NO: 82644.

The probes having the nucleotide sequence represented by SEQ ID NOS: 164571, 164572, 164573, 164574, 164575, 164576, 164577, 164578, 164579, 164580, 164581 were selected from the ORFs having the nucleotide sequence represented by SEQ ID NO: 82645.

The probes having the nucleotide sequence represented by SEQ ID NOS: 164582, 164583, 164584, 164585, 164586, 164587, 164588, 164589, 164590, 164591, 164592 were selected from the ORFs having the nucleotide sequence represented by SEQ ID NO: 82646.

The probes having the nucleotide sequence represented by SEQ ID NOS: 164593, 164594, 164595, 164596, 164597, 164598, 164599, 164600, 164601, 164602, 164603 were selected from the ORFs having the nucleotide sequence represented by SEQ ID NO: 82647.

The probes having the nucleotide sequence represented by SEQ ID NOS: 164604, 164605, 164606, 164607, 164608, 164609, 164610, 164611, 164612, 164613, 164614 were selected from the ORFs having the nucleotide sequence represented by SEQ ID NO: 82648.

The probes having the nucleotide sequence represented by SEQ ID NOS: 164615, 164616, 164617, 164618, 164619, 164620, 164621, 164622, 164623, 164624, 164625 were selected from the ORFs having the nucleotide sequence represented by SEQ ID NO: 82649.

The probes having the nucleotide sequence represented by SEQ ID NOS: 164626, 164627, 164628, 164629, 164630, 164631, 164632, 164633, 164634, 164635, 164636 were selected from the ORFs having the nucleotide sequence represented by SEQ ID NO: 82650.

The probes having the nucleotide sequence represented by SEQ ID NOS: 164637, 164638, 164639, 164640, 164641, 164642, 164643, 164644, 164645, 164646, 164647 were selected from the ORFs having the nucleotide sequence represented by SEQ ID NO: 82651.

The probes having the nucleotide sequence represented by SEQ ID NOS: 164648, 164649, 164650, 164651, 164652, 164653, 164654, 164655, 164656, 164657, 164658 were selected from the ORFs having the nucleotide sequence represented by SEQ ID NO: 82652.

The probes having the nucleotide sequence represented by SEQ ID NOS: 164659, 164660, 164661, 164662, 164663, 164664, 164665, 164666, 164667, 164668, 164669 were selected from the ORFs having the nucleotide sequence represented by SEQ ID NO: 82653.

The probes having the nucleotide sequence represented by SEQ ID NOS: 164670, 164671, 164672, 164673, 164674, 164675, 164676, 164677, 164678, 164679, 164680 were selected from the ORFs having the nucleotide sequence represented by SEQ ID NO: 82654.

The probes having the nucleotide sequence represented by SEQ ID NOS: 164681, 164682, 164683, 164684, 164685, 164686, 164687, 164688, 164689, 164690, 164691 were selected from the ORFs having the nucleotide sequence represented by SEQ ID NO: 82655.

The probes having the nucleotide sequence represented by SEQ ID NOS: 164692, 164693, 164694, 164695, 164696, 164697, 164698, 164699, 164700, 164701, 164702 were selected from the ORFs having the nucleotide sequence represented by SEQ ID NO: 82656.

The probes having the nucleotide sequence represented by SEQ ID NOS: 164703, 164704, 164705, 164706, 164707, 164708, 164709, 164710, 164711, 164712, 164713 were selected from the ORFs having the nucleotide sequence represented by SEQ ID NO: 82657.

The probes having the nucleotide sequence represented by SEQ ID NOS: 164714, 164715, 164716, 164717, 164718, 164719, 164720, 164721, 164722, 164723, 164724 were selected from the ORFs having the nucleotide sequence represented by SEQ ID NO: 82658.

The probes having the nucleotide sequence represented by SEQ ID NOS: 164725, 164726, 164727, 164728, 164729, 164730, 164731, 164732, 164733, 164734, 164735 were selected from the ORFs having the nucleotide sequence represented by SEQ ID NO: 82659.

The probes having the nucleotide sequence represented by SEQ ID NOS: 164736, 164737, 164738, 164739, 164740, 164741, 164742, 164743, 164744, 164745, 164746 were selected from the ORFs having the nucleotide sequence represented by SEQ ID NO: 82660.

The probes having the nucleotide sequence represented by SEQ ID NOS: 164747, 164748, 164749, 164750, 164751, 164752, 164753, 164754, 164755, 164756, 164757 were selected from the ORFs having the nucleotide sequence represented by SEQ ID NO: 82661.

The probes having the nucleotide sequence represented by SEQ ID NOS: 164758, 164759, 164760, 164761, 164762, 164763, 164764, 164765, 164766, 164767, 164768 were selected from the ORFs having the nucleotide sequence represented by SEQ ID NO: 82662.

The probes having the nucleotide sequence represented by SEQ ID NOS: 164769, 164770, 164771, 164772, 164773, 164774, 164775, 164776, 164777, 164778, 164779 were selected from the ORFs having the nucleotide sequence represented by SEQ ID NO: 82663.

The probes having the nucleotide sequence represented by SEQ ID NOS: 164780, 164781, 164782, 164783, 164784, 164785, 164786, 164787, 164788, 164789, 164790 were selected from the ORFs having the nucleotide sequence represented by SEQ ID NO: 82664.

The probes having the nucleotide sequence represented by SEQ ID NOS: 164791, 164792, 164793, 164794, 164795, 164796, 164797, 164798, 164799, 164800, 164801 were selected from the ORFs having the nucleotide sequence represented by SEQ ID NO: 82665.

The probes having the nucleotide sequence represented by SEQ ID NOS: 164802, 164803, 164804, 164805, 164806, 164807, 164808, 164809, 164810, 164811, 164812 were selected from the ORFs having the nucleotide sequence represented by SEQ ID NO: 82666.

The probes having the nucleotide sequence represented by SEQ ID NOS: 164813, 164814, 164815, 164816, 164817, 164818, 164819, 164820, 164821, 164822, 164823 were selected from the ORFs having the nucleotide sequence represented by SEQ ID NO: 82667.

The probes having the nucleotide sequence represented by SEQ ID NOS: 164824, 164825, 164826, 164827, 164828, 164829, 164830, 164831, 164832, 164833, 164834 were selected from the ORFs having the nucleotide sequence represented by SEQ ID NO: 82668.

The probes having the nucleotide sequence represented by SEQ ID NOS: 164835, 164836, 164837, 164838, 164839, 164840, 164841, 164842, 164843, 164844, 164845 were selected from the ORFs having the nucleotide sequence represented by SEQ ID NO: 82669.

The probes having the nucleotide sequence represented by SEQ ID NOS: 164846, 164847, 164848, 164849, 164850, 164851, 164852, 164853, 164854, 164855, 164856 were selected from the ORFs having the nucleotide sequence represented by SEQ ID NO: 82670.

The probes having the nucleotide sequence represented by SEQ ID NOS: 164857, 164858, 164859, 164860, 164861, 164862, 164863, 164864, 164865, 164866, 164867 were selected from the ORFs having the nucleotide sequence represented by SEQ ID NO: 82671.

The probes having the nucleotide sequence represented by SEQ ID NOS: 164868, 164869, 164870, 164871, 164872, 164873, 164874, 164875, 164876, 164877, 164878 were selected from the ORFs having the nucleotide sequence represented by SEQ ID NO: 82672.

The probes having the nucleotide sequence represented by SEQ ID NOS: 164879, 164880, 164881, 164882, 164883, 164884, 164885, 164886, 164887, 164888, 164889 were selected from the ORFs having the nucleotide sequence represented by SEQ ID NO: 82673.

The probes having the nucleotide sequence represented by SEQ ID NOS: 164890, 164891, 164892, 164893, 164894, 164895, 164896, 164897, 164898, 164899, 164900 were selected from the ORFs having the nucleotide sequence represented by SEQ ID NO: 82674.

The probes having the nucleotide sequence represented by SEQ ID NOS: 164901, 164902, 164903, 164904, 164905, 164906, 164907, 164908, 164909, 164910, 164911 were selected from the ORFs having the nucleotide sequence represented by SEQ ID NO: 82675.

The probes having the nucleotide sequence represented by SEQ ID NOS: 164912, 164913, 164914, 164915, 164916, 164917, 164918, 164919, 164920, 164921, 164922 were selected from the ORFs having the nucleotide sequence represented by SEQ ID NO: 82676.

The probes having the nucleotide sequence represented by SEQ ID NOS: 164923, 164924, 164925, 164926, 164927, 164928, 164929, 164930, 164931, 164932, 164933 were selected from the ORFs having the nucleotide sequence represented by SEQ ID NO: 82677.

The probes having the nucleotide sequence represented by SEQ ID NOS: 164934, 164935, 164936, 164937, 164938, 164939, 164940, 164941, 164942, 164943, 164944 were selected from the ORFs having the nucleotide sequence represented by SEQ ID NO: 82678.

The probes having the nucleotide sequence represented by SEQ ID NOS: 164945, 164946, 164947, 164948, 164949, 164950, 164951, 164952, 164953, 164954, 164955 were selected from the ORFs having the nucleotide sequence represented by SEQ ID NO: 82679.

The probes having the nucleotide sequence represented by SEQ ID NOS: 164956, 164957, 164958, 164959, 164960, 164961, 164962, 164963, 164964, 164965, 164966 were selected from the ORFs having the nucleotide sequence represented by SEQ ID NO: 82680.

The probes having the nucleotide sequence represented by SEQ ID NOS: 164967, 164968, 164969, 164970, 164971, 164972, 164973, 164974, 164975, 164976, 164977 were selected from the ORFs having the nucleotide sequence represented by SEQ ID NO: 82681.

The probes having the nucleotide sequence represented by SEQ ID NOS: 164978, 164979, 164980, 164981, 164982, 164983, 164984, 164985, 164986, 164987, 164988 were selected from the ORFs having the nucleotide sequence represented by SEQ ID NO: 82682.

The probes having the nucleotide sequence represented by SEQ ID NOS: 164989, 164990, 164991, 164992, 164993, 164994, 164995, 164996, 164997, 164998, 164999 were selected from the ORFs having the nucleotide sequence represented by SEQ ID NO: 82683.

The probes having the nucleotide sequence represented by SEQ ID NOS: 165000, 165001, 165002, 165003, 165004, 165005, 165006, 165007, 165008, 165009, 165010 were selected from the ORFs having the nucleotide sequence represented by SEQ ID NO: 82684.

The probes having the nucleotide sequence represented by SEQ ID NOS: 165011, 165012, 165013, 165014, 165015, 165016, 165017, 165018, 165019, 165020, 165021 were selected from the ORFs having the nucleotide sequence represented by SEQ ID NO: 82685.

The probes having the nucleotide sequence represented by SEQ ID NOS: 165022, 165023, 165024, 165025, 165026, 165027, 165028, 165029, 165030, 165031, 165032 were selected from the ORFs having the nucleotide sequence represented by SEQ ID NO: 82686.

The probes having the nucleotide sequence represented by SEQ ID NOS: 165033, 165034, 165035, 165036, 165037, 165038, 165039, 165040, 165041, 165042, 165043 were selected from the ORFs having the nucleotide sequence represented by SEQ ID NO: 82687.

The probes having the nucleotide sequence represented by SEQ ID NOS: 165044, 165045, 165046, 165047, 165048, 165049, 165050, 165051, 165052, 165053, 165054 were selected from the ORFs having the nucleotide sequence represented by SEQ ID NO: 82688.

The probes having the nucleotide sequence represented by SEQ ID NOS: 165055, 165056, 165057, 165058, 165059, 165060, 165061, 165062, 165063, 165064, 165065 were selected from the ORFs having the nucleotide sequence represented by SEQ ID NO: 82689.

The probes having the nucleotide sequence represented by SEQ ID NOS: 165066, 165067, 165068, 165069, 165070, 165071, 165072, 165073, 165074, 165075, 165076 were selected from the ORFs having the nucleotide sequence represented by SEQ ID NO: 82690.

The probes having the nucleotide sequence represented by SEQ ID NOS: 165077, 165078, 165079, 165080, 165081, 165082, 165083, 165084, 165085, 165086, 165087 were selected from the ORFs having the nucleotide sequence represented by SEQ ID NO: 82691.

The probes having the nucleotide sequence represented by SEQ ID NOS: 165088, 165089, 165090, 165091, 165092, 165093, 165094, 165095, 165096, 165097, 165098 were selected from the ORFs having the nucleotide sequence represented by SEQ ID NO: 82692.

The probes having the nucleotide sequence represented by SEQ ID NOS: 165099, 165100, 165101, 165102, 165103, 165104, 165105, 165106, 165107, 165108, 165109 were selected from the ORFs having the nucleotide sequence represented by SEQ ID NO: 82693.

The probes having the nucleotide sequence represented by SEQ ID NOS: 165110, 165111, 165112, 165113, 165114, 165115,. 165116, 165117, 165118, 165119, 165120 were selected from the ORFs having the nucleotide sequence represented by SEQ ID NO: 82694.

The probes having the nucleotide sequence represented by SEQ ID NOS: 165121, 165122, 165123, 165124, 165125, 165126, 165127, 165128, 165129, 165130, 165131 were selected from the ORFs having the nucleotide sequence represented by SEQ ID NO: 82695.

The probes having the nucleotide sequence represented by SEQ ID NOS: 165132, 165133, 165134, 165135, 165136, 165137, 165138, 165139, 165140, 165141, 165142 were selected from the ORFs having the nucleotide sequence represented by SEQ ID NO: 82696.

The probes having the nucleotide sequence represented by SEQ ID NOS: 165143, 165144, 165145, 165146, 165147, 165148, 165149, 165150, 165151, 165152, 165153 were selected from the ORFs having the nucleotide sequence represented by SEQ ID NO: 82697.

The probes having the nucleotide sequence represented by SEQ ID NOS: 165154, 165155, 165156, 165157, 165158, 165159, 165160, 165161, 165162, 165163, 165164 were selected from the ORFs having the nucleotide sequence represented by SEQ ID NO: 82698.

The probes having the nucleotide sequence represented by SEQ ID NOS: 165165, 165166, 165167, 165168, 165169, 165170, 165171, 165172, 165173, 165174, 165175 were selected from the ORFs having the nucleotide sequence represented by SEQ ID NO: 82699.

The probes having the nucleotide sequence represented by SEQ ID NOS: 165176, 165177, 165178, 165179, 165180, 165181, 165182, 165183, 165184, 165185, 165186 were selected from the ORFs having the nucleotide sequence represented by SEQ ID NO: 82700.

The probes having the nucleotide sequence represented by SEQ ID NOS: 165187, 165188, 165189, 165190, 165191, 165192, 165193, 165194, 165195, 165196, 165197 were selected from the ORFs having the nucleotide sequence represented by SEQ ID NO: 82701.

The probes having the nucleotide sequence represented by SEQ ID NOS: 165198, 165199, 165200, 165201, 165202, 165203, 165204, 165205, 165206, 165207, 165208 were selected from the ORFs having the nucleotide sequence represented by SEQ ID NO: 82702.

The probes having the nucleotide sequence represented by SEQ ID NOS: 165209, 165210, 165211, 165212, 165213, 165214, 165215, 165216, 165217, 165218, 1.65219, 165220, 165221, 165222, 165223, 165224, 165225, 165226, 165227, 165228, 165229, 165230 were selected from the ORFs having the nucleotide sequence represented by SEQ ID NO: 82703.

The probes having the nucleotide sequence represented by SEQ ID NOS: 165231, 165232, 165233, 165234, 165235, 165236, 165237, 165238, 165239, 165240, 165241 were selected from the ORFs having the nucleotide sequence represented by SEQ ID NO: 82704.

The probes having the nucleotide sequence represented by SEQ ID NOS: 165242, 165243, 165244, 165245, 165246, 165247, 165248, 165249, 165250, 165251, 165252, 165253, 165254, 165255, 165256, 165257, 165258, 165259, 165260, 165261, 165262, 165263 were selected from the ORFs having the nucleotide sequence represented by SEQ ID NO: 82705.

The probes having the nucleotide sequence represented by SEQ ID NOS: 165264, 165265, 165266, 165267, 165268, 165269, 165270, 165271, 165272, 165273, 165274 were selected from the ORFs having the nucleotide sequence represented by SEQ ID NO: 82706.

The probes having the nucleotide sequence represented by SEQ ID NOS: 165275, 165276, 165277, 165278, 165279, 165280, 165281, 165282, 165283, 165284, 165285 were selected from the ORFs having the nucleotide sequence represented by SEQ ID NO: 82707.

The probes having the nucleotide sequence represented by SEQ ID NOS: 165286, 165287, 165288, 165289, 165290, 165291, 165292, 165293, 165294, 165295, 165296 were selected from the ORFs having the nucleotide sequence represented by SEQ ID NO: 82708.

The probes having the nucleotide sequence represented by SEQ ID NOS: 165297, 165298, 165299, 165300, 165301, 165302, 165303, 165304, 165305, 165306, 165307 were selected from the ORFs having the nucleotide sequence represented by SEQ ID NO: 82709.

The probes having the nucleotide sequence represented by SEQ ID NOS: 165308, 165309, 165310, 165311, 165312, 165313, 165314, 165315, 165316, 165317, 165318 were selected from the ORFs having the nucleotide sequence represented by SEQ ID NO: 82710.

The probes having the nucleotide sequence represented by SEQ ID NOS: 165319, 165320, 165321, 165322, 165323, 165324, 165325, 165326, 165327, 165328, 165329 were selected from the ORFs having the nucleotide sequence represented by SEQ ID NO: 82711.

The probes having the nucleotide sequence represented by SEQ ID NOS: 165330, 165331, 165332, 165333, 165334, 165335, 165336, 165337, 165338, 165339, 165340 were selected from the ORFs having the nucleotide sequence represented by SEQ ID NO: 82712.

The probes having the nucleotide sequence represented by SEQ ID NOS: 165341, 165342, 165343, 165344, 165345, 165346, 165347, 165348, 165349, 165350, 165351 were selected from the ORFs having the nucleotide sequence represented by SEQ ID NO: 82713.

The probes having the nucleotide sequence represented by SEQ ID NOS: 165352, 165353, 165354, 165355, 165356, 165357, 165358, 165359, 165360, 165361, 165362 were selected from the ORFs having the nucleotide sequence represented by SEQ ID NO: 82714.

The probes having the nucleotide sequence represented by SEQ ID NOS: 165363, 165364, 165365, 165366, 165367, 165368, 165369, 165370, 165371, 165372, 165373 were selected from the ORFs having the nucleotide sequence represented by SEQ ID NO: 82715.

The probes having the nucleotide sequence represented by SEQ ID NOS: 165374, 165375, 165376, 165377, 165378, 165379, 165380, 165381, 165382, 165383 were selected from the ORFs having the nucleotide sequence represented by SEQ ID NO: 82716.

The probes having the nucleotide sequence represented by SEQ ID NOS: 165384, 165385, 165386, 165387, 165388, 165389, 165390, 165391, 165392, 165393, 165394 were selected from the ORFs having the nucleotide sequence represented by SEQ ID NO: 82717.

The probes having the nucleotide sequence represented by SEQ ID NOS: 165395, 165396, 165397, 165398, 165399, 165400, 165401, 165402, 165403, 165404, 165405 were selected from the ORFs having the nucleotide sequence represented by SEQ ID NO: 82718.

The probes having the nucleotide sequence represented by SEQ ID NOS: 165406, 165407, 165408, 165409, 165410, 165411, 165412, 165413, 165414, 165415, 165416 were selected from the ORFs having the nucleotide sequence represented by SEQ ID NO: 82719.

The probes having the nucleotide sequence represented by SEQ ID NOS: 165417, 165418, 165419, 165420, 165421, 165422, 165423, 165424, 165425, 165426, 165427 were selected from the ORFs having the nucleotide sequence represented by SEQ ID NO: 82720.

The probes having the nucleotide sequence represented by SEQ ID NOS: 165428, 165429, 165430, 165431, 165432, 165433, 165434, 165435, 165436, 165437, 165438 were selected from the ORFs having the nucleotide sequence represented by SEQ ID NO: 82721.

The probes having the nucleotide sequence represented by SEQ ID NOS: 165439, 165440, 165441, 165442, 165443, 165444, 165445, 165446, 165447, 165448, 165449 were selected from the ORFs having the nucleotide sequence represented by SEQ ID NO: 82722.

The probes having the nucleotide sequence represented by SEQ ID NOS: 165450, 165451, 165452, 165453, 165454, 165455, 165456, 165457, 165458, 165459, 165460 were selected from the ORFs having the nucleotide sequence represented by SEQ ID NO: 82723.

The probes having the nucleotide sequence represented by SEQ ID NOS: 165461, 165462, 165463, 165464, 165465, 165466, 165467, 165468, 165469, 165470, 165471 were selected from the ORFs having the nucleotide sequence represented by SEQ ID NO: 82724.

The probes having the nucleotide sequence represented by SEQ ID NOS: 165472, 165473, 165474, 165475, 165476, 165477, 165478, 165479, 165480, 165481, 165482 were selected from the ORFs having the nucleotide sequence represented by SEQ ID NO: 82725.

The probes having the nucleotide sequence represented by SEQ ID NOS: 165483, 165484, 165485, 165486, 165487, 165488, 165489, 165490, 165491, 165492, 165493 were selected from the ORFs having the nucleotide sequence represented by SEQ ID NO: 82726.

The probes having the nucleotide sequence represented by SEQ ID NOS: 165494, 165495, 165496, 165497, 165498, 165499, 165500, 165501, 165502, 165503, 165504 were selected from the ORFs having the nucleotide sequence represented by SEQ ID NO: 82727.

The probes having the nucleotide sequence represented by SEQ ID NOS: 165505, 165506, 165507, 165508, 165509, 165510, 165511, 165512, 165513, 165514, 165515 were selected from the ORFs having the nucleotide sequence represented by SEQ ID NO: 82728.

The probes having the nucleotide sequence represented by SEQ ID NOS: 165516, 165517, 165518, 165519, 165520, 165521, 165522, 165523, 165524, 165525, 165526 were selected from the ORFs having the nucleotide sequence represented by SEQ ID NO: 82729.

The probes having the nucleotide sequence represented by SEQ ID NOS: 165527, 165528, 165529, 165530, 165531, 165532, 165533, 165534, 165535, 165536, 165537 were selected from the ORFs having the nucleotide sequence represented by SEQ ID NO: 82730.

The probes having the nucleotide sequence represented by SEQ ID NOS: 165538, 165539, 165540, 165541, 165542, 165543, 165544, 165545, 165546, 165547, 165548 were selected from the ORFs having the nucleotide sequence represented by SEQ ID NO: 82731.

The probes having the nucleotide sequence represented by SEQ ID NOS: 165549, 165550, 165551, 165552, 165553, 165554, 165555, 165556, 165557, 165558, 165559 were selected from the ORFs having the nucleotide sequence represented by SEQ ID NO: 82732.

The probes having the nucleotide sequence represented by SEQ ID NOS: 165560, 165561, 165562, 165563, 165564, 165565, 165566, 165567, 165568, 165569, 165570 were selected from the ORFs having the nucleotide sequence represented by SEQ ID NO: 82733.

The probes having the nucleotide sequence represented by SEQ ID NOS: 165571, 165572, 165573, 165574, 165575, 165576, 165577, 165578, 165579, 165580, 165581 were selected from the ORFs having the nucleotide sequence represented by SEQ ID NO: 82734.

The probes having the nucleotide sequence represented by SEQ ID NOS: 165582, 165583, 165584, 165585, 165586, 165587, 165588, 165589, 165590, 165591, 165592 were selected from the ORFs having the nucleotide sequence represented by SEQ ID NO: 82735.

The probes having the nucleotide sequence represented by SEQ ID NOS: 165593, 165594, 165595, 165596, 165597, 165598, 165599, 165600, 165601, 165602, 165603 were selected from the ORFs having the nucleotide sequence represented by SEQ ID NO: 82736.

The probes having the nucleotide sequence represented by SEQ ID NOS: 165604, 165605, 165606, 165607, 165608, 165609, 165610, 165611, 165612, 165613, 165614 were selected from the ORFs having the nucleotide sequence represented by SEQ ID NO: 82737.

The probes having the nucleotide sequence represented by SEQ ID NOS: 165615, 165616, 165617, 165618, 165619, 165620, 165621, 165622, 165623, 165624, 165625 were selected from the ORFs having the nucleotide sequence represented by SEQ ID NO: 82738.

The probes having the nucleotide sequence represented by SEQ ID NOS: 165626, 165627, 165628, 165629, 165630, 165631, 165632, 165633, 165634, 165635, 165636 were selected from the ORFs having the nucleotide sequence represented by SEQ ID NO: 82739.

The probes having the nucleotide sequence represented by SEQ ID NOS: 165637, 165638, 165639, 165640, 165641, 165642, 165643, 165644, 165645, 165646, 165647 were selected from the ORFs having the nucleotide sequence represented by SEQ ID NO: 82740.

The probes having the nucleotide sequence represented by SEQ ID NOS: 165648, 165649, 165650, 165651, 165652, 165653, 165654, 165655, 165656, 165657, 165658 were selected from the ORFs having the nucleotide sequence represented by SEQ ID NO: 82741.

The probes having the nucleotide sequence represented by SEQ ID NOS: 165659, 165660, 165661, 165662, 165663, 165664, 165665, 165666, 165667, 165668, 165669 were selected from the ORFs having the nucleotide sequence represented by SEQ ID NO: 82742.

The probes having the nucleotide sequence represented by SEQ ID NOS: 165670, 165671, 165672, 165673, 165674, 165675, 165676, 165677, 165678, 165679, 165680 were selected from the ORFs having the nucleotide sequence represented by SEQ ID NO: 82743.

The probes having the nucleotide sequence represented by SEQ ID NOS: 165681, 165682, 165683, 165684, 165685, 165686, 165687, 165688, 165689, 165690, 165691 were selected from the ORFs having the nucleotide sequence represented by SEQ ID NO: 82744.

The probes having the nucleotide sequence represented by SEQ ID NOS: 165692, 165693, 165694, 165695, 165696, 165697, 165698, 165699, 165700, 165701, 165702 were selected from the ORFs having the nucleotide sequence represented by SEQ ID NO: 82745.

The probes having the nucleotide sequence represented by SEQ ID NOS: 165703, 165704, 165705, 165706, 165707, 165708, 165709, 165710, 165711, 165712, 165713 were selected from the ORFs having the nucleotide sequence represented by SEQ ID NO: 82746.

The probes having the nucleotide sequence represented by SEQ ID NOS: 165714, 165715, 165716, 165717, 165718, 165719, 165720, 165721, 165722, 165723, 165724 were selected from the ORFs having the nucleotide sequence represented by SEQ ID NO: 82747.

The probes having the nucleotide sequence represented by SEQ ID NOS: 165725, 165726, 165727, 165728, 165729, 165730, 165731, 165732, 165733, 165734, 165735 were selected from the ORFs having the nucleotide sequence represented by SEQ ID NO: 82748.

The probes having the nucleotide sequence represented by SEQ ID NOS: 165736, 165737, 165738, 165739, 165740, 165741, 165742, 165743, 165744, 165745, 165746 were selected from the ORFs having the nucleotide sequence represented by SEQ ID NO: 82749.

The probes having the nucleotide sequence represented by SEQ ID NOS: 165747, 165748, 165749, 165750, 165751, 165752, 165753, 165754, 165755, 165756, 165757 were selected from the ORFs having the nucleotide sequence represented by SEQ ID NO: 82750.

The probes having the nucleotide sequence represented by SEQ ID NOS: 165758, 165759, 165760, 165761, 165762, 165763, 165764, 165765, 165766, 165767, 165768 were selected from the ORFs having the nucleotide sequence represented by SEQ ID NO: 82751.

The probes having the nucleotide sequence represented by SEQ ID NOS: 165769, 165770, 165771, 165772, 165773, 165774, 165775, 165776, 165777, 165778, 165779 were selected from the ORFs having the nucleotide sequence represented by SEQ ID NO: 82752.

The probes having the nucleotide sequence represented by SEQ ID NOS: 165780, 165781, 165782, 165783, 165784, 165785, 165786, 165787, 165788, 165789, 165790 were selected from the ORFs having the nucleotide sequence represented by SEQ ID NO: 82753.

The probes having the nucleotide sequence represented by SEQ ID NOS: 165791, 165792, 165793, 165794, 165795, 165796, 165797, 165798, 165799, 165800, 165801 were selected from the ORFs having the nucleotide sequence represented by SEQ ID NO: 82754.

The probes having the nucleotide sequence represented by SEQ ID NOS: 165802, 165803, 165804, 165805, 165806, 165807, 165808, 165809, 165810, 165811, 165812 were selected from the ORFs having the nucleotide sequence represented by SEQ ID NO: 82755.

The probes having the nucleotide sequence represented by SEQ ID NOS: 165813, 165814, 165815, 165816, 165817, 165818, 165819, 165820, 165821, 165822, 165823 were selected from the ORFs having the nucleotide sequence represented by SEQ ID NO: 82756.

The probes having the nucleotide sequence represented by SEQ ID NOS: 165824, 165825, 165826, 165827, 165828, 165829, 165830, 165831, 165832, 165833, 165834 were selected from the ORFs having the nucleotide sequence represented by SEQ ID NO: 82757.

The probes having the nucleotide sequence represented by SEQ ID NOS: 165835, 165836, 165837, 165838, 165839, 165840, 165841, 165842, 165843, 165844, 165845, 165846, 165847, 165848, 165849, 165850, 165851, 165852, 165853, 165854, 165855, 165856 were selected from the ORFs having the nucleotide sequence represented by SEQ ID NO: 82758.

The probes having the nucleotide sequence represented by SEQ ID NOS: 165857, 165858, 165859, 165860, 165861, 165862, 165863, 165864, 165865, 165866, 165867 were selected from the ORFs having the nucleotide sequence represented by SEQ ID NO: 82759.

The probes having the nucleotide sequence represented by SEQ ID NOS: 165868, 165869, 165870, 165871, 165872, 165873, 165874, 165875, 165876, 165877, 165878 were selected from the ORFs having the nucleotide sequence represented by SEQ ID NO: 82760.

The probes having the nucleotide sequence represented by SEQ ID NOS: 165879, 165880, 165881, 165882, 165883, 165884, 165885, 165886, 165887, 165888, 165889 were selected from the ORFs having the nucleotide sequence represented by SEQ ID NO: 82761.

The probes having the nucleotide sequence represented by SEQ ID NOS: 165890, 165891, 165892, 165893, 165894, 165895, 165896, 165897, 165898, 165899, 165900 were selected from the ORFs having the nucleotide sequence represented by SEQ ID NO: 82762.

The probes having the nucleotide sequence represented by SEQ ID NOS: 165901, 165902, 165903, 165904, 165905, 165906, 165907, 165908, 165909, 165910, 165911, 165912, 165913, 165914, 165915, 165916, 165917, 165918, 165919, 165920, 165921, 165922 were selected from the ORFs having the nucleotide sequence represented by SEQ ID NO: 82763.

The probes having the nucleotide sequence represented by SEQ ID NOS: 165923, 165924, 165925, 165926, 165927, 165928, 165929, 165930, 165931, 165932, 165933 were selected from the ORFs having the nucleotide sequence represented by SEQ ID NO: 82764.

The probes having the nucleotide sequence represented by SEQ ID NOS: 165934, 165935, 165936, 165937, 165938, 165939, 165940, 165941, 165942, 165943, 165944 were selected from the ORFs having the nucleotide sequence represented by SEQ ID NO: 82765.

The probes having the nucleotide sequence represented by SEQ ID NOS: 165945, 165946, 165947, 165948, 165949, 165950, 165951, 165952, 165953, 165954, 165955 were selected from the ORFs having the nucleotide sequence represented by SEQ ID NO: 82766.

The probes having the nucleotide sequence represented by SEQ ID NOS: 165956, 165957, 165958, 165959, 165960, 165961, 165962, 165963, 165964, 165965, 165966 were selected from the ORFs having the nucleotide sequence represented by SEQ ID NO: 82767.

The probes having the nucleotide sequence represented by SEQ ID NOS: 165967, 165968, 165969, 165970, 165971, 165972, 165973, 165974, 165975, 165976, 165977 were selected from the ORFs having the nucleotide sequence represented by SEQ ID NO: 82768.

The probes having the nucleotide sequence represented by SEQ ID NOS: 165978, 165979, 165980, 165981, 165982, 165983, 165984, 165985, 165986, 165987, 165988 were selected from the ORFs having the nucleotide sequence represented by SEQ ID NO: 82769.

The probes having the nucleotide sequence represented by SEQ ID NOS: 165989, 165990, 165991, 165992, 165993, 165994, 165995, 165996, 165997, 165998, 165999 were selected from the ORFs having the nucleotide sequence represented by SEQ ID NO: 82770.

The probes having the nucleotide sequence represented by SEQ ID NOS: 166000, 166001, 166002, 166003, 166004, 166005, 166006, 166007, 166008, 166009, 166010 were selected from the ORFs having the nucleotide sequence represented by SEQ ID NO: 82771.

The probes having the nucleotide sequence represented by SEQ ID NOS: 166011, 166012, 166013, 166014, 166015, 166016, 166017, 166018, 166019, 166020, 166021 were selected from the ORFs having the nucleotide sequence represented by SEQ ID NO: 82772.

The probes having the nucleotide sequence represented by SEQ ID NOS: 166022, 166023, 166024, 166025, 166026, 166027, 166028, 166029, 166030, 166031, 166032 were selected from the ORFs having the nucleotide sequence represented by SEQ ID NO: 82773.

The probes having the nucleotide sequence represented by SEQ ID NOS: 166033, 166034, 166035, 166036, 166037, 166038, 166039, 166040, 166041, 166042, 166043 were selected from the ORFs having the nucleotide sequence represented by SEQ ID NO: 82774.

The probes having the nucleotide sequence represented by SEQ ID NOS: 166044, 166045, 166046, 166047, 166048, 166049, 166050, 166051, 166052, 166053, 166054 were selected from the ORFs having the nucleotide sequence represented by SEQ ID NO: 82775.

The probes having the nucleotide sequence represented by SEQ ID NOS: 166055, 166056, 166057, 166058, 166059, 166060, 166061, 166062, 166063, 166064, 166065 were selected from the ORFs having the nucleotide sequence represented by SEQ ID NO: 82776.

The probes having the nucleotide sequence represented by SEQ ID NOS: 166066, 166067, 166068, 166069, 166070, 166071, 166072, 166073, 166074, 166075, 166076 were selected from the ORFs having the nucleotide sequence represented by SEQ ID NO: 82777.

The probes having the nucleotide sequence represented by SEQ ID NOS: 166077, 166078, 166079, 166080, 166081, 166082, 166083, 166084, 166085, 166086, 166087 were selected from the ORFs having the nucleotide sequence represented by SEQ ID NO: 82778.

The probes having the nucleotide sequence represented by SEQ ID NOS: 166088, 166089, 166090, 166091, 166092, 166093, 166094, 166095, 166096, 166097, 166098 were selected from the ORFs having the nucleotide sequence represented by SEQ ID NO: 82779.

The probes having the nucleotide sequence represented by SEQ ID NOS: 166099, 166100, 166101, 166102, 166103, 166104, 166105, 166106, 166107, 166108, 166109 were selected from the ORFs having the nucleotide sequence represented by SEQ ID NO: 82780.

The probes having the nucleotide sequence represented by SEQ ID NOS: 166110, 166111, 166112, 166113, 166114, 166115, 166116, 166117, 166118, 166119, 166120 were selected from the ORFs having the nucleotide sequence represented by SEQ ID NO: 82781.

The probes having the nucleotide sequence represented by SEQ ID NOS: 166121, 166122, 166123, 166124, 166125, 166126, 166127, 166128, 166129, 166130, 166131 were selected from the ORFs having the nucleotide sequence represented by SEQ ID NO: 82782.

The probes having the nucleotide sequence represented by SEQ ID NOS: 166132, 166133, 166134, 166135, 166136, 166137, 166138, 166139, 166140, 166141, 166142 were selected from the ORFs having the nucleotide sequence represented by SEQ ID NO: 82783.

The probes having the nucleotide sequence represented by SEQ ID NOS: 166143, 166144, 166145, 166146, 166147, 166148, 166149, 166150, 166151, 166152, 166153 were selected from the ORFs having the nucleotide sequence represented by SEQ ID NO: 82784.

The probes from 28 DNA sequences of mitochondrial ORFs described as above (1-C) were as follows:

The probes having the nucleotide sequence represented by SEQ ID NOS: 166182, 166183, 166184, 166185, 166186, 166187, 166188, 166189, 166190, 166191, 166192 were selected from the ORFs having the nucleotide sequence represented by SEQ ID NO: 166154.

The probes having the nucleotide sequence represented by SEQ ID NOS: 166193, 166194, 166195, 166196, 166197, 166198, 166199, 166200, 166201, 166202, 166203 were selected from the ORFs having the nucleotide sequence represented by SEQ ID NO: 166155.

The probes having the nucleotide sequence represented by SEQ ID NOS: 166204, 166205, 166206, 166207, 166208, 166209, 166210, 166211, 166212, 166213, 166214 were selected from the ORFs having the nucleotide sequence represented by SEQ ID NO: 166156.

The probes having the nucleotide sequence represented by SEQ ID NOS: 166215, 166216, 166217, 166218, 166219, 166220, 166221, 166222, 166223, 166224, 166225 were selected from the ORFs having the nucleotide sequence represented by SEQ ID NO: 166157.

The probes having the nucleotide sequence represented by SEQ ID NOS: 166226, 166227, 166228, 166229, 166230, 166231, 166232, 166233, 166234, 166235, 166236 were selected from the ORFs having the nucleotide sequence represented by SEQ ID NO: 166158.

The probes having the nucleotide sequence represented by SEQ ID NOS: 166237, 166238, 166239, 166240, 166241, 166242, 166243, 166244, 166245, 166246, 166247 were selected from the ORFs having the nucleotide sequence represented by SEQ ID NO: 166159.

The probes having the nucleotide sequence represented by SEQ ID NOS: 166248, 166249, 166250, 166251, 166252, 166253, 166254, 166255, 166256, 166257, 166258 were selected from the ORFs having the nucleotide sequence represented by SEQ ID NO: 166160.

The probes having the nucleotide sequence represented by SEQ ID NOS: 166259, 166260, 166261, 166262, 166263, 166264,

166265, 166266, 166267, 166268, 166269 were selected from the ORFs having the nucleotide sequence represented by SEQ ID NO: 166161.

The probes having the nucleotide sequence represented by SEQ ID NOS: 166270, 166271, 166272, 166273, 166274, 166275, 166276, 166277, 166278, 166279, 166280 were selected from the ORFs having the nucleotide sequence represented by SEQ ID NO: 166162.

The probes having the nucleotide sequence represented by SEQ ID NOS: 166281, 166282, 166283, 166284, 166285, 166286, 166287, 166288, 166289, 166290, 166291 were selected from the ORFs having the nucleotide sequence represented by SEQ ID NO: 166163.

The probes having the nucleotide sequence represented by SEQ ID NOS: 166292, 166293, 166294, 166295, 166296, 166297, 166298, 166299, 166300, 166301, 166302 were selected from the ORFs having the nucleotide sequence represented by SEQ ID NO: 166164.

The probes having the nucleotide sequence represented by SEQ ID NOS: 166303, 166304, 166305, 166306, 166307, 166308, 166309, 166310, 166311, 166312, 166313 were selected from the ORFs having the nucleotide sequence represented by SEQ ID NO: 166165.

The probes having the nucleotide sequence represented by SEQ ID NOS: 166314, 166315, 166316, 166317, 166318, 166319, 166320, 166321, 166322, 166323, 166324 were selected from the ORFs having the nucleotide sequence represented by SEQ ID NO: 166166.

The probes having the nucleotide sequence represented by SEQ ID NOS: 166325, 166326, 166327, 166328, 166329, 166330, 166331, 166332, 166333, 166334, 166335 were selected from the ORFs having the nucleotide sequence represented by SEQ ID NO: 166167.

The probes having the nucleotide sequence represented by SEQ ID NOS: 166336, 166337, 166338, 166339, 166340, 166341, 166342, 166343, 166344, 166345, 166346 were selected from the ORFs having the nucleotide sequence represented by SEQ ID NO: 166168.

The probes having the nucleotide sequence represented by SEQ ID NOS: 166347, 166348, 166349, 166350, 166351, 166352, 166353, 166354, 166355, 166356, 166357 were selected from the ORFs having the nucleotide sequence represented by SEQ ID NO: 166169.

The probes having the nucleotide sequence represented by SEQ ID NOS: 166358, 166359, 166360, 166361, 166362, 166363, 166364, 166365, 166366, 166367, 166368 were selected from the ORFs having the nucleotide sequence represented by SEQ ID NO: 166170.

The probes having the nucleotide sequence represented by SEQ ID NOS: 166369, 166370, 166371, 166372, 166373, 166374, 166375, 166376, 166377, 166378, 166379 were selected from the ORFs having the nucleotide sequence represented by SEQ ID NO: 166171.

The probes having the nucleotide sequence represented by SEQ ID NOS: 166380, 166381, 166382, 166383, 166384, 166385, 166386, 166387, 166388, 166389, 166390 were selected from the ORFs having the nucleotide sequence represented by SEQ ID NO: 166172.

The probes having the nucleotide sequence represented by SEQ ID NOS: 166391, 166392, 166393, 166394, 166395, 166396, 166397, 166398, 166399, 166400, 166401 were selected from the ORFs having the nucleotide sequence represented by SEQ ID NO: 166173

The probes having the nucleotide sequence represented by SEQ ID NOS: 166402, 166403, 166404, 166405, 166406, 166407, 166408, 166409, 166410, 166411, 166412 were selected from the ORFs having the nucleotide sequence represented by SEQ ID NO: 166174.

The probes having the nucleotide sequence represented by SEQ ID NOS: 166413, 166414, 166415, 166416, 166417, 166418, 166419, 166420, 166421, 166422, 166423 were selected from the ORFs having the nucleotide sequence represented by SEQ ID NO: 166175.

The probes having the nucleotide sequence represented by SEQ ID NOS: 166424, 166425, 166426, 166427, 166428, 166429, 166430, 166431, 166432, 166433, 166434 were selected from the ORFs having the nucleotide sequence represented by SEQ ID NO: 166176.

The probes having the nucleotide sequence represented by SEQ ID NOS: 166435, 166436, 166437, 166438, 166439, 166440, 166441, 166442, 166443, 166444, 166445 were selected from the ORFs having the nucleotide sequence represented by SEQ ID NO: 166177.

The probes having the nucleotide sequence represented by SEQ ID NOS: 166446, 166447, 166448, 166449, 166450, 166451, 166452, 166453, 166454, 166455, 166456 were selected from the ORFs having the nucleotide sequence represented by SEQ ID NO: 166178.

The probes having the nucleotide sequence represented by SEQ ID NOS: 166457, 166458, 166459, 166460, 166461, 166462, 166463, 166464, 166465, 166466, 166467 were selected from the ORFs having the nucleotide sequence represented by SEQ ID NO: 166179.

The probes having the nucleotide sequence represented by SEQ ID NOS: 166468, 166469, 166470, 166471, 166472, 166473, 166474, 166475, 166476, 166477, 166478 were selected from the ORFs having the nucleotide sequence represented by SEQ ID NO: 166180.

The probes having the nucleotide sequence represented by SEQ ID NOS: 166479, 166480, 166481, 166482, 166483, 166484, 166485, 166486, 166487, 166488, 166489 were selected from the ORFs having the nucleotide sequence represented by SEQ ID NO: 166181.

The probes from 553 DNA sequences described as above (1-D) were as follows:

The probes having the nucleotide sequence represented by SEQ ID NOS: 167043, 167044, 167045, 167046, 167047, 167048, 167049, 167050, 167051, 167052, 167053 were selected from the ORFs having the nucleotide sequence represented by SEQ ID NO: 166490.

The probes having the nucleotide sequence represented by SEQ ID NOS: 167054, 167055, 167056, 167057, 167058, 167059, 167060, 167061, 167062, 167063, 167064 were selected from the ORFs having the nucleotide sequence represented by SEQ ID NO: 16649.

The probes having the nucleotide sequence represented by SEQ ID NOS: 167065, 167066, 167067, 167068, 167069, 167070, 167071, 167072, 167073, 167074, 167075 were selected from the ORFs having the nucleotide sequence represented by SEQ ID NO: 166492.

The probes having the nucleotide sequence represented by SEQ ID NOS: 167076, 167077, 167078, 167079, 167080, 167081, 167082, 167083, 167084, 167085, 167086 were selected from the ORFs having the nucleotide sequence represented by SEQ ID NO: 166493.

The probes having the nucleotide sequence represented by SEQ ID NOS: 167087, 167088, 167089, 167090, 167091, 167092, 167093, 167094, 167095, 167096, 167097 were selected from the ORFs having the nucleotide sequence represented by SEQ ID NO: 166494.

The probes having the nucleotide sequence represented by SEQ ID NOS: 167098, 167099, 167100, 167101, 167102, 167103, 167104, 167105, 167106, 167107, 167108 were selected from the ORFs having the nucleotide sequence represented by SEQ ID NO: 166495.

The probes having the nucleotide sequence represented by SEQ ID NOS: 167109, 167110, 167111, 167112, 167113, 167114, 167115, 167116, 167117, 167118, 167119 were selected from the ORFs having the nucleotide sequence represented by SEQ ID NO: 166496.

The probes having the nucleotide sequence represented by SEQ ID NOS: 167120, 167121, 167122, 167123, 167124, 167125, 167126, 167127, 167128, 167129, 167130 were selected from the ORFs having the nucleotide sequence represented by SEQ ID NO: 166497.

The probes having the nucleotide sequence represented by SEQ ID NOS: 167131, 167132, 167133, 167134, 167135, 167136, 167137, 167138, 167139, 167140, 167141 were selected from the ORFs having the nucleotide sequence represented by SEQ ID NO: 166498.

The probes having the nucleotide sequence represented by SEQ ID NOS: 167142, 167143, 167144, 167145, 167146, 167147, 167148, 167149, 167150, 167151, 167152 were selected from the ORFs having the nucleotide sequence represented by SEQ ID NO: 166499.

The probes having the nucleotide sequence represented by SEQ ID NOS: 167153, 167154, 167155, 167156, 167157, 167158, 167159, 167160, 167161, 167162, 167163 were selected from the ORFs having the nucleotide sequence represented by SEQ ID NO: 166500.

The probes having the nucleotide sequence represented by SEQ ID NOS: 167164, 167165, 167166, 167167, 167168, 167169, 167170, 167171, 167172, 167173, 167174 were selected from the ORFs having the nucleotide sequence represented by SEQ ID NO: 166501.

The probes having the nucleotide sequence represented by SEQ ID NOS: 167175, 167176, 167177, 167178, 167179, 167180, 167181, 167182, 167183, 167184, 167185 were selected from the ORFs having the nucleotide sequence represented by SEQ ID NO: 166502.

The probes having the nucleotide sequence represented by SEQ ID NOS: 167186, 167187, 167188, 167189, 167190, 167191, 167192, 167193, 167194, 167195, 167196 were selected from the ORFs having the nucleotide sequence represented by SEQ ID NO: 166503.

The probes having the nucleotide sequence represented by SEQ ID NOS: 167197, 167198, 167199, 167200, 167201, 167202, 167203, 167204, 167205, 167206, 167207 were selected from the ORFs having the nucleotide sequence represented by SEQ ID NO: 166504.

The probes having the nucleotide sequence represented by SEQ ID NOS: 167208, 167209, 167210, 167211, 167212, 167213, 167214, 167215, 167216, 167217, 167218 were selected from the ORFs having the nucleotide sequence represented by SEQ ID NO: 166505.

The probes having the nucleotide sequence represented by SEQ ID NOS: 167219, 167220, 167221, 167222, 167223, 167224, 167225, 167226, 167227, 167228, 167229 were selected from the ORFs having the nucleotide sequence represented by SEQ ID NO: 166506.

The probes having the nucleotide sequence represented by SEQ ID NOS: 167230, 167231, 167232, 167233, 167234, 167235, 167236, 167237, 167238, 167239, 167240 were selected from the ORFs having the nucleotide sequence represented by SEQ ID NO: 166507.

The probes having the nucleotide sequence represented by SEQ ID NOS: 167241, 167242, 167243, 167244, 167245, 167246, 167247, 167248, 167249, 167250, 167251 were selected from the ORFs having the nucleotide sequence represented by SEQ ID NO: 166508.

The probes having the nucleotide sequence represented by SEQ ID NOS: 167252, 167253, 167254, 167255, 167256, 167257, 167258, 167259, 167260, 167261, 167262 were selected from the ORFs having the nucleotide sequence represented by SEQ ID NO: 166509.

The probes having the nucleotide sequence represented by SEQ ID NOS: 167263, 167264, 167265, 167266, 167267, 167268, 167269, 167270, 167271, 167272, 167273 were selected from the ORFs having the nucleotide sequence represented by SEQ ID NO: 166510.

The probes having the nucleotide sequence represented by SEQ ID NOS: 167274, 167275, 167276, 167277, 167278, 167279, 167280, 167281, 167282, 167283, 167284 were selected from the ORFs having the nucleotide sequence represented by SEQ ID NO: 166511.

The probes having the nucleotide sequence represented by SEQ ID NOS: 167285, 167286, 167287, 167288, 167289, 167290, 167291, 167292, 167293, 167294, 167295 were selected from the ORFs having the nucleotide sequence represented by SEQ ID NO: 166512.

The probes having the nucleotide sequence represented by SEQ ID NOS: 167296, 167297, 167298, 167299, 167300, 167301, 167302, 167303, 167304, 167305, 167306 were selected from the ORFs having the nucleotide sequence represented by SEQ ID NO: 166513.

The probes having the nucleotide sequence represented by SEQ ID NOS: 167307, 167308, 167309, 167310, 167311, 167312, 167313, 167314, 167315, 167316, 167317 were selected from the ORFs having the nucleotide sequence represented by SEQ ID NO: 166514.

The probes having the nucleotide sequence represented by SEQ ID NOS: 167318, 167319, 167320, 167321, 167322, 167323, 167324, 167325, 167326, 167327, 167328 were selected from the ORFs having the nucleotide sequence represented by SEQ ID NO: 166515.

The probes having the nucleotide sequence represented by SEQ ID NOS: 167329, 167330, 167331, 167332, 167333, 167334, 167335, 167336, 167337, 167338, 167339 were selected from the ORFs having the nucleotide sequence represented by SEQ ID NO: 166516.

The probes having the nucleotide sequence represented by SEQ ID NOS: 167340, 167341, 167342, 167343, 167344, 167345, 167346, 167347, 167348, 167349, 167350 were selected from the ORFs having the nucleotide sequence represented by SEQ ID NO: 166517.

The probes having the nucleotide sequence represented by SEQ ID NOS: 167351, 167352, 167353, 167354, 167355, 167356, 167357, 167358, 167359, 167360, 167361 were selected from the ORFs having the nucleotide sequence represented by SEQ ID NO: 166518.

The probes having the nucleotide sequence represented by SEQ ID NOS: 167362, 167363, 167364, 167365, 167366, 167367, 167368, 167369, 167370, 167371, 167372 were selected from the ORFs having the nucleotide sequence represented by SEQ ID NO: 166519.

The probes having the nucleotide sequence represented by SEQ ID NOS: 167373, 167374, 167375, 167376, 167377, 167378, 167379, 167380, 167381, 167382, 167383 were selected from the ORFs having the nucleotide sequence represented by SEQ ID NO: 166520.

The probes having the nucleotide sequence represented by SEQ ID NOS: 167384, 167385, 167386, 167387, 167388, 167389, 167390, 167391, 167392, 167393, 167394 were selected from the ORFs having the nucleotide sequence represented by SEQ ID NO: 166521.

The probes having the nucleotide sequence represented by SEQ ID NOS: 167395, 167396, 167397, 167398, 167399, 167400, 167401, 167402, 167403, 167404, 167405 were selected from the ORFs having the nucleotide sequence represented by SEQ ID NO: 166522.

The probes having the nucleotide sequence represented by SEQ ID NOS: 167406, 167407, 167408, 167409, 167410, 167411, 167412, 167413, 167414, 167415, 167416 were selected from the ORFs having the nucleotide sequence represented by SEQ ID NO: 166523.

The probes having the nucleotide sequence represented by SEQ ID NOS: 167417, 167418, 167419, 167420, 167421, 167422, 167423, 167424, 167425, 167426, 167427 were selected from the ORFs having the nucleotide sequence represented by SEQ ID NO: 166524.

The probes having the nucleotide sequence represented by SEQ ID NOS: 167428, 167429, 167430, 167431, 167432, 167433, 167434, 167435, 167436, 167437, 167438 were selected from the ORFs having the nucleotide sequence represented by SEQ ID NO: 166525.

The probes having the nucleotide sequence represented by SEQ ID NOS: 167439, 167440, 167441, 167442, 167443, 167444, 167445, 167446, 167447, 167448, 167449 were selected from the ORFs having the nucleotide sequence represented by SEQ ID NO: 166526.

The probes having the nucleotide sequence represented by SEQ ID NOS: 167450, 167451, 167452, 167453, 167454, 167455, 167456, 167457, 167458, 167459, 167460 were selected from the ORFs having the nucleotide sequence represented by SEQ ID NO: 166527.

The probes having the nucleotide sequence represented by SEQ ID NOS: 167461, 167462, 167463, 167464, 167465, 167466, 167467, 167468, 167469, 167470, 167471 were selected from the ORFs having the nucleotide sequence represented by SEQ ID NO: 166528.

The probes having the nucleotide sequence represented by SEQ ID NOS: 167472, 167473, 167474, 167475, 167476, 167477, 167478, 167479, 167480, 167481, 167482 were selected from the ORFs having the nucleotide sequence represented by SEQ ID NO: 166529.

The probes having the nucleotide sequence represented by SEQ ID NOS: 167483, 167484, 167485, 167486, 167487, 167488, 167489, 167490, 167491, 167492, 167493 were selected from the ORFs having the nucleotide sequence represented by SEQ ID NO: 166530.

The probes having the nucleotide sequence represented by SEQ ID NOS: 167494, 167495, 167496, 167497, 167498, 167499, 167500, 167501, 167502, 167503, 167504 were selected from the ORFs having the nucleotide sequence represented by SEQ ID NO: 166531.

The probes having the nucleotide sequence represented by SEQ ID NOS: 167505, 167506, 167507, 167508, 167509, 167510, 167511, 167512, 167513, 167514, 167515 were selected from the ORFs having the nucleotide sequence represented by SEQ ID NO: 166532.

The probes having the nucleotide sequence represented by SEQ ID NOS: 167516, 167517, 167518, 167519, 167520, 167521, 167522, 167523, 167524, 167525, 167526 were selected from the ORFs having the nucleotide sequence represented by SEQ ID NO: 166533.

The probes having the nucleotide sequence represented by SEQ ID NOS: 167527, 167528, 167529, 167530, 167531, 167532, 167533, 167534, 167535, 167536, 167537 were selected from the ORFs having the nucleotide sequence represented by SEQ ID NO: 166534.

The probes having the nucleotide sequence represented by SEQ ID NOS: 167538, 167539, 167540, 167541, 167542, 167543, 167544, 167545, 167546, 167547, 167548 were selected from the ORFs having the nucleotide sequence represented by SEQ ID NO: 166535.

The probes having the nucleotide sequence represented by SEQ ID NOS: 167549, 167550, 167551, 167552, 167553, 167554, 167555, 167556, 167557, 167558, 167559 were selected from the ORFs having the nucleotide sequence represented by SEQ ID NO: 166536.

The probes having the nucleotide sequence represented by SEQ ID NOS: 167560, 167561, 167562, 167563, 167564, 167565, 167566, 167567, 167568, 167569, 167570 were selected from the ORFs having the nucleotide sequence represented by SEQ ID NO: 166537.

The probes having the nucleotide sequence represented by SEQ ID NOS: 167571, 167572, 167573, 167574, 167575, 167576, 167577, 167578, 167579, 167580, 167581 were selected from the ORFs having the nucleotide sequence represented by SEQ ID NO: 166538.

The probes having the nucleotide sequence represented by SEQ ID NOS: 167582, 167583, 167584, 167585, 167586, 167587, 167588, 167589, 167590, 167591, 167592 were selected from the ORFs having the nucleotide sequence represented by SEQ ID NO: 166539.

The probes having the nucleotide sequence represented by SEQ ID NOS: 167593, 167594, 167595, 167596, 167597, 167598, 167599, 167600, 167601, 167602, 167603 were selected from the ORFs having the nucleotide sequence represented by SEQ ID NO: 166540.

The probes having the nucleotide sequence represented by SEQ ID NOS: 167604, 167605, 167606, 167607, 167608, 167609, 167610, 167611, 167612, 167613, 167614 were selected from the ORFs having the nucleotide sequence represented by SEQ ID NO: 166541.

The probes having the nucleotide sequence represented by SEQ ID NOS: 167615, 167616, 167617, 167618, 167619, 167620, 167621, 167622, 167623, 167624, 167625 were selected from the ORFs having the nucleotide sequence represented by SEQ ID NO: 166542.

The probes having the nucleotide sequence represented by SEQ ID NOS: 167626, 167627, 167628, 167629, 167630, 167631, 167632, 167633, 167634, 167635, 167636 were selected from the ORFs having the nucleotide sequence represented by SEQ ID NO: 166543.

The probes having the nucleotide sequence represented by SEQ ID NOS: 167637, 167638, 167639, 167640, 167641, 167642, 167643, 167644, 167645, 167646, 167647 were selected from the ORFs having the nucleotide sequence represented by SEQ ID NO: 166544.

The probes having the nucleotide sequence represented by SEQ ID NOS: 167648, 167649, 167650, 167651, 167652, 167653, 167654, 167655, 167656, 167657, 167658 were selected from the ORFs having the nucleotide sequence represented by SEQ ID NO: 166545.

The probes having the nucleotide sequence represented by SEQ ID NOS: 167659, 167660, 167661, 167662, 167663, 167664, 167665, 167666, 167667, 167668, 167669 were selected from the ORFs having the nucleotide sequence represented by SEQ ID NO: 166546.

The probes having the nucleotide sequence represented by SEQ ID NOS: 167670, 167671, 167672, 167673, 167674, 167675, 167676, 167677, 167678, 167679, 167680 were selected from the ORFs having the nucleotide sequence represented by SEQ ID NO: 166547.

The probes having the nucleotide sequence represented by SEQ ID NOS: 167681, 167682, 167683, 167684, 167685, 167686, 167687, 167688, 167689, 167690, 167691 were selected from the ORFs having the nucleotide sequence represented by SEQ ID NO: 166548.

The probes having the nucleotide sequence represented by SEQ ID NOS: 167692, 167693, 167694, 167695, 167696, 167697, 167698, 167699, 167700, 167701, 167702 were selected from the ORFs having the nucleotide sequence represented by SEQ ID NO: 166549.

The probes having the nucleotide sequence represented by SEQ ID NOS: 167703, 167704, 167705, 167706, 167707, 167708, 167709, 167710, 167711, 167712, 167713 were selected from the ORFs having the nucleotide sequence represented by SEQ ID NO: 166550.

The probes having the nucleotide sequence represented by SEQ ID NOS: 167714, 167715, 167716, 167717, 167718, 167719, 167720, 167721, 167722, 167723, 167724 were selected from the ORFs having the nucleotide sequence represented by SEQ ID NO: 166551.

The probes having the nucleotide sequence represented by SEQ ID NOS: 167725, 167726, 167727, 167728, 167729, 167730, 167731, 167732, 167733, 167734, 167735 were selected from the ORFs having the nucleotide sequence represented by SEQ ID NO: 166552.

The probes having the nucleotide sequence represented by SEQ ID NOS: 167736, 167737, 167738, 167739, 167740, 167741, 167742, 167743, 167744, 167745, 167746 were selected from the ORFs having the nucleotide sequence represented by SEQ ID NO: 166553.

The probes having the nucleotide sequence represented by SEQ ID NOS: 167747, 167748, 167749, 167750, 167751, 167752, 167753, 167754, 167755, 167756, 167757 were selected from the ORFs having the nucleotide sequence represented by SEQ ID NO: 166554.

The probes having the nucleotide sequence represented by SEQ ID NOS: 167758, 167759, 167760, 167761, 167762, 167763, 167764, 167765, 167766, 167767, 167768 were selected from the ORFs having the nucleotide sequence represented by SEQ ID NO: 166555.

The probes having the nucleotide sequence represented by SEQ ID NOS: 167769, 167770, 167771, 167772, 167773, 167774, 167775, 167776, 167777, 167778, 167779 were selected from the ORFs having the nucleotide sequence represented by SEQ ID NO: 166556.

The probes having the nucleotide sequence represented by SEQ ID NOS: 167780, 167781, 167782, 167783, 167784, 167785, 167786, 167787, 167788, 167789, 167790 were selected from the ORFs having the nucleotide sequence represented by SEQ ID NO: 166557.

The probes having the nucleotide sequence represented by SEQ ID NOS: 167791, 167792, 167793, 167794, 167795, 167796, 167797, 167798, 167799, 167800, 167801 were selected from the ORFs having the nucleotide sequence represented by SEQ ID NO: 166558.

The probes having the nucleotide sequence represented by SEQ ID NOS: 167802, 167803, 167804, 167805, 167806, 167807, 167808, 167809, 167810, 167811, 167812 were selected from the ORFs having the nucleotide sequence represented by SEQ ID NO: 166559.

The probes having the nucleotide sequence represented by SEQ ID NOS: 167813, 167814, 167815, 167816, 167817, 167818, 167819, 167820, 167821, 167822, 167823 were selected from the ORFs having the nucleotide sequence represented by SEQ ID NO: 166560.

The probes having the nucleotide sequence represented by SEQ ID NOS: 167824, 167825, 167826, 167827, 167828, 167829, 167830, 167831, 167832, 167833, 167834 were selected from the ORFs having the nucleotide sequence represented by SEQ ID NO: 166561.

The probes having the nucleotide sequence represented by SEQ ID NOS: 167835, 167836, 167837, 167838, 167839, 167840, 167841, 167842, 167843, 167844, 167845 were selected from the ORFs having the nucleotide sequence represented by SEQ ID NO: 166562.

The probes having the nucleotide sequence represented by SEQ ID NOS: 167846, 167847, 167848, 167849, 167850, 167851, 167852, 167853, 167854, 167855, 167856 were selected from the ORFs having the nucleotide sequence represented by SEQ ID NO: 166563.

The probes having the nucleotide sequence represented by SEQ ID NOS: 167857, 167858, 167859, 167860, 167861, 167862, 167863, 167864, 167865, 167866, 167867 were selected from the ORFs having the nucleotide sequence represented by SEQ ID NO: 166564.

The probes having the nucleotide sequence represented by SEQ ID NOS: 167868, 167869, 167870, 167871, 167872, 167873, 167874, 167875, 167876, 167877, 167878 were selected from the ORFs having the nucleotide sequence represented by SEQ ID NO: 166565.

The probes having the nucleotide sequence represented by SEQ ID NOS: 167879, 167880, 167881, 167882, 167883, 167884, 167885, 167886, 167887, 167888, 167889 were selected from the ORFs having the nucleotide sequence represented by SEQ ID NO: 166566.

The probes having the nucleotide sequence represented by SEQ ID NOS: 167890, 167891, 167892, 167893, 167894, 167895, 167896, 167897, 167898, 167899, 167900 were selected from the ORFs having the nucleotide sequence represented by SEQ ID NO: 166567.

The probes having the nucleotide sequence represented by SEQ ID NOS: 167901, 167902, 167903, 167904, 167905, 167906, 167907, 167908, 167909, 167910, 167911 were selected from the ORFs having the nucleotide sequence represented by SEQ ID NO: 166568.

The probes having the nucleotide sequence represented by SEQ ID NOS: 167912, 167913, 167914, 167915, 167916, 167917, 167918, 167919, 167920, 167921, 167922 were selected from the ORFs having the nucleotide sequence represented by SEQ ID NO: 166569.

The probes having the nucleotide sequence represented by SEQ ID NOS: 167923, 167924, 167925, 167926, 167927, 167928, 167929, 167930, 167931, 167932, 167933 were selected from the ORFs having the nucleotide sequence represented by SEQ ID NO: 166570.

The probes having the nucleotide sequence represented by SEQ ID NOS: 167934, 167935, 167936, 167937, 167938, 167939, 167940, 167941, 167942, 167943, 167944 were selected from the ORFs having the nucleotide sequence represented by SEQ ID NO: 166571.

The probes having the nucleotide sequence represented by SEQ ID NOS: 167945, 167946, 167947, 167948, 167949, 167950, 167951, 167952, 167953, 167954, 167955 were selected from the ORFs having the nucleotide sequence represented by SEQ ID NO: 166572.

The probes having the nucleotide sequence represented by SEQ ID NOS: 167956, 167957, 167958, 167959, 167960, 167961, 167962, 167963, 167964, 167965, 167966 were selected from the ORFs having the nucleotide sequence represented by SEQ ID NO: 166573.

The probes having the nucleotide sequence represented by SEQ ID NOS: 167967, 167968, 167969, 167970, 167971, 167972, 167973, 167974, 167975, 167976, 167977 were selected from the ORFs having the nucleotide sequence represented by SEQ ID NO: 166574.

The probes having the nucleotide sequence represented by SEQ ID NOS: 167978, 167979, 167980, 167981, 167982, 167983, 167984, 167985, 167986, 167987, 167988 were selected from the ORFs having the nucleotide sequence represented by SEQ ID NO: 166575.

The probes having the nucleotide sequence represented by SEQ ID NOS: 167989, 167990, 167991, 167992, 167993, 167994, 167995, 167996, 167997, 167998, 167999 were selected from the ORFs having the nucleotide sequence represented by SEQ ID NO: 166576.

The probes having the nucleotide sequence represented by SEQ ID NOS: 168000, 168001, 168002, 168003, 168004, 168005, 168006, 168007, 168008, 168009, 168010 were selected from the ORFs having the nucleotide sequence represented by SEQ ID NO: 166577.

The probes having the nucleotide sequence represented by SEQ ID NOS: 168011, 168012, 168013, 168014, 168015, 168016, 168017, 168018, 168019, 168020, 168021 were selected from the ORFs having the nucleotide sequence represented by SEQ ID NO: 166578.

The probes having the nucleotide sequence represented by SEQ ID NOS: 168022, 168023, 168024, 168025, 168026, 168027, 168028, 168029, 168030, 168031, 168032 were selected from the ORFs having the nucleotide sequence represented by SEQ ID NO: 166579.

The probes having the nucleotide sequence represented by SEQ ID NOS: 168033, 168034, 168035, 168036, 168037, 168038, 168039, 168040, 168041, 168042, 168043 were selected from the ORFs having the nucleotide sequence represented by SEQ ID NO: 166580.

The probes having the nucleotide sequence represented by SEQ ID NOS: 168044, 168045, 168046, 168047, 168048, 168049, 168050, 168051, 168052, 168053, 168054 were selected from the ORFs having the nucleotide sequence represented by SEQ ID NO: 166581.

The probes having the nucleotide sequence represented by SEQ ID NOS: 168055, 168056, 168057, 168058, 168059, 168060, 168061, 168062, 168063, 168064, 168065 were selected from the ORFs having the nucleotide sequence represented by SEQ ID NO: 166582.

The probes having the nucleotide sequence represented by SEQ ID NOS: 168066, 168067, 168068, 168069, 168070, 168071, 168072, 168073, 168074, 168075, 168076 were selected from the ORFs having the nucleotide sequence represented by SEQ ID NO: 166583.

The probes having the nucleotide sequence represented by SEQ ID NOS: 168077, 168078, 168079, 168080, 168081, 168082, 168083, 168084, 168085, 168086, 168087 were selected from the ORFs having the nucleotide sequence represented by SEQ ID NO: 166584.

The probes having the nucleotide sequence represented by SEQ ID NOS: 168088, 168089, 168090, 168091, 168092, 168093, 168094, 168095, 168096, 168097, 168098 were selected from the ORFs having the nucleotide sequence represented by SEQ ID NO: 166585.

The probes having the nucleotide sequence represented by SEQ ID NOS: 168099, 168100, 168101, 168102, 168103, 168104, 168105, 168106, 168107, 168108, 168109 were selected from the ORFs having the nucleotide sequence represented by SEQ ID NO: 166586.

The probes having the nucleotide sequence represented by SEQ ID NOS: 168110, 168111, 168112, 168113, 168114, 168115, 168116, 168117, 168118, 168119, 168120 were selected from the ORFs having the nucleotide sequence represented by SEQ ID NO: 166587.

The probes having the nucleotide sequence represented by SEQ ID NOS: 168121, 168122, 168123, 168124, 168125, 168126, 168127, 168128, 168129, 168130, 168131 were selected from the ORFs having the nucleotide sequence represented by. SEQ ID NO: 166588.

The probes having the nucleotide sequence represented by SEQ ID NOS: 168132, 168133, 168134, 168135, 168136, 168137, 168138, 168139, 168140, 168141, 168142 were selected from the ORFs having the nucleotide sequence represented by SEQ ID NO: 166589.

The probes having the nucleotide sequence represented by SEQ ID NOS: 168143, 168144, 168145, 168146, 168147, 168148, 168149, 168150, 168151, 168152, 168153 were selected from the ORFs having the nucleotide sequence represented by SEQ ID NO: 166590.

The probes having the nucleotide sequence represented by SEQ ID NOS: 168154, 168155, 168156, 168157, 168158, 168159, 168160, 168161, 168162, 168163, 168164 were selected from he ORFs having the nucleotide sequence represented by SEQ ID NO: 166591.

The probes having the nucleotide sequence represented by SEQ ID NOS: 168165, 168166, 168167, 168168, 168169, 168170, 168171, 168172, 168173, 168174, 168175 were selected from the ORFs having the nucleotide sequence represented by SEQ ID NO: 166592.

The probes having the nucleotide sequence represented by SEQ ID NOS: 168176, 168177, 168178, 168179, 168180, 168181, 168182, 168183, 168184, 168185, 168186 were selected from the ORFs having the nucleotide sequence represented by SEQ ID NO: 166593.

The probes having the nucleotide sequence represented by SEQ ID NOS: 168187, 168188, 168189, 168190, 168191, 168192, 168193, 168194, 168195, 168196, 168197 were selected from the ORFs having the nucleotide sequence represented by SEQ ID NO: 166594.

The probes having the nucleotide sequence represented by SEQ ID NOS: 168198, 168199, 168200, 168201, 168202, 168203, 168204, 168205, 168206, 168207, 168208 were selected from the ORFs having the nucleotide sequence represented by SEQ ID NO: 166595.

The probes having the nucleotide sequence represented by SEQ ID NOS: 168209, 168210, 168211, 168212, 168213, 168214, 168215, 168216, 168217, 168218, 168219 were selected from the ORFs having the nucleotide sequence represented by SEQ ID NO: 166596.

The probes having the nucleotide sequence represented by SEQ ID NOS: 168220, 168221, 168222, 168223, 168224, 168225, 168226, 168227, 168228, 168229, 168230 were selected from the ORFs having the nucleotide sequence represented by SEQ ID NO: 166597.

The probes having the nucleotide sequence represented by SEQ ID NOS: 168231, 168232, 168233, 168234, 168235, 168236, 168237, 168238, 168239, 168240, 168241 were selected from the ORFs having the nucleotide sequence represented by SEQ ID NO: 166598.

The probes having the nucleotide sequence represented by SEQ ID NOS: 168242, 168243, 168244, 168245, 168246, 168247, 168248, 168249, 168250, 168251, 168252 were selected from the ORFs having the nucleotide sequence represented by SEQ ID NO: 166599.

The probes having the nucleotide sequence represented by SEQ ID NOS: 168253, 168254, 168255, 168256, 168257, 168258, 168259, 168260, 168261, 168262, 168263 were selected from the ORFs having the nucleotide sequence represented by SEQ ID NO: 166600.

The probes having the nucleotide sequence represented by SEQ ID NOS: 168264, 168265, 168266, 168267, 168268, 168269, 168270, 168271, 168272, 168273, 168274 were selected from the ORFs having the nucleotide sequence represented by SEQ ID NO: 166601.

The probes having the nucleotide sequence represented by SEQ ID NOS: 168275, 168276, 168277, 168278, 168279, 168280, 168281, 168282, 168283, 168284, 168285 were selected from the ORFs having the nucleotide sequence represented by SEQ ID NO: 166602.

The probes having the nucleotide sequence represented by SEQ ID NOS: 168286, 168287, 168288, 168289, 168290, 168291, 168292, 168293, 168294, 168295, 168296 were selected from the ORFs having the nucleotide sequence represented by SEQ ID NO: 166603.

The probes having the nucleotide sequence represented by SEQ ID NOS: 168297, 168298, 168299, 168300, 168301, 168302, 168303, 168304, 168305, 168306, 168307 were selected from the ORFs having the nucleotide sequence represented by SEQ ID NO: 166604.

The probes having the nucleotide sequence represented by SEQ ID NOS: 168308, 168309, 168310, 168311, 168312, 168313, 168314, 168315, 168316, 168317, 168318 were selected from the ORFs having the nucleotide sequence represented by SEQ ID NO: 166605.

The probes having the nucleotide sequence represented by SEQ ID NOS: 168319, 168320, 168321, 168322, 168323, 168324, 168325, 168326, 168327, 168328, 168329 were selected from the ORFs having the nucleotide sequence represented by SEQ ID NO: 166606.

The probes having the nucleotide sequence represented by SEQ ID NOS: 168330, 168331, 168332, 168333, 168334, 168335, 168336, 168337, 168338, 168339, 168340 were selected from the ORFs having the nucleotide sequence represented by SEQ ID NO: 166607.

The probes having the nucleotide sequence represented by SEQ ID NOS: 168341, 168342, 168343, 168344, 168345, 168346, 168347, 168348, 168349, 168350, 168351 were selected from the ORFs having the nucleotide sequence represented by SEQ ID NO: 166608.

The probes having the nucleotide sequence represented by SEQ ID NOS: 168352, 168353, 168354, 168355, 168356, 168357, 168358, 168359, 168360, 168361, 168362 were selected from the ORFs having the nucleotide sequence represented by SEQ ID NO: 166609.

The probes having the nucleotide sequence represented by SEQ ID NOS: 168363, 168364, 168365, 168366, 168367, 168368, 168369, 168370, 168371, 168372, 168373 were selected from the ORFs having the nucleotide sequence represented by SEQ ID NO: 166610.

The probes having the nucleotide sequence represented by SEQ ID NOS: 168374, 168375, 168376, 168377, 168378, 168379, 168380, 168381, 168382, 168383, 168384 were selected from the ORFs having the nucleotide sequence represented by SEQ ID NO: 166611.

The probes having the nucleotide sequence represented by SEQ ID NOS: 168385, 168386, 168387, 168388, 168389, 168390, 168391, 168392, 168393, 168394, 168395 were selected from the ORFs having the nucleotide sequence represented by SEQ ID NO: 166612.

The probes having the nucleotide sequence represented by SEQ ID NOS: 168396, 168397, 168398, 168399, 168400, 168401, 168402, 168403, 168404, 168405, 168406 were selected from the ORFs having the nucleotide sequence represented by SEQ ID NO: 166613.

The probes having the nucleotide sequence represented by SEQ ID NOS: 168407, 168408, 168409, 168410, 168411, 168412, 168413, 168414, 168415, 168416, 168417 were selected from the ORFs having the nucleotide sequence represented by SEQ ID NO: 166614.

The probes having the nucleotide sequence represented by SEQ ID NOS: 168418, 168419, 168420, 168421, 168422, 168423, 168424, 168425, 168426, 168427, 168428 were selected from the ORFs having the nucleotide sequence represented by SEQ ID NO: 166615.

The probes having the nucleotide sequence represented by SEQ ID NOS: 168429, 168430, 168431, 168432, 168433, 168434, 168435, 168436, 168437, 168438, 168439 were selected from the ORFs having the nucleotide sequence represented by SEQ ID NO: 166616.

The probes having the nucleotide sequence represented by SEQ ID NOS: 168440, 168441, 168442, 168443, 168444, 168445, 168446, 168447, 168448, 168449, 168450 were selected from the ORFs having the nucleotide sequence represented by SEQ ID NO: 166617.

The probes having the nucleotide sequence represented by SEQ ID NOS: 168451, 168452, 168453, 168454, 168455, 168456, 168457, 168458, 168459, 168460, 168461 were selected from the ORFs having the nucleotide sequence represented by SEQ ID NO: 166618.

The probes having the nucleotide sequence represented by SEQ ID NOS: 168462, 168463, 168464, 168465, 168466, 168467, 168468, 168469, 168470, 168471, 168472 were selected from the ORFs having the nucleotide sequence represented by SEQ ID NO: 166619.

The probes having the nucleotide sequence represented by SEQ ID NOS: 168473, 168474, 168475, 168476, 168477, 168478, 168479, 168480, 168481, 168482, 168483 were selected from the ORFs having the nucleotide sequence represented by SEQ ID NO: 166620.

The probes having the nucleotide sequence represented by SEQ ID NOS: 168484, 168485, 168486, 168487, 168488, 168489, 168490, 168491, 168492, 168493, 168494 were selected from the ORFs having the nucleotide sequence represented by SEQ ID NO: 166621.

The probes having the nucleotide sequence represented by SEQ ID NOS: 168495, 168496, 168497, 168498, 168499, 168500, 168501, 168502, 168503, 168504, 168505 were selected from the ORFs having the nucleotide sequence represented by SEQ ID NO: 166622.

The probes having the nucleotide sequence represented by SEQ ID NOS: 168506, 168507, 168508, 168509, 168510, 168511, 168512, 168513, 168514, 168515, 168516 were selected from the ORFs having the nucleotide sequence represented by SEQ ID NO: 166623.

The probes having the nucleotide sequence represented by SEQ ID NOS: 168517, 168518, 168519, 168520, 168521, 168522, 168523, 168524, 168525, 168526, 168527 were selected from the ORFs having the nucleotide sequence represented by SEQ ID NO: 166624.

The probes having the nucleotide sequence represented by SEQ ID NOS: 168528, 168529, 168530, 168531, 168532, 168533, 168534, 168535, 168536, 168537, 168538 were selected from the ORFs having the nucleotide sequence represented by SEQ ID NO: 166625.

The probes having the nucleotide sequence represented by SEQ ID NOS: 168539, 168540, 168541, 168542, 168543, 168544, 168545, 168546, 168547, 168548, 168549 were selected from the ORFs having the nucleotide sequence represented by SEQ ID NO: 166626.

The probes having the nucleotide sequence represented by SEQ ID NOS: 168550, 168551, 168552, 168553, 168554, 168555, 168556, 168557, 168558, 168559, 168560 were selected from the ORFs having the nucleotide sequence represented by SEQ ID NO: 166627.

The probes having the nucleotide sequence represented by SEQ ID NOS: 168561, 168562, 168563, 168564, 168565, 168566, 168567, 168568, 168569, 168570, 168571 were selected from the ORFs having the nucleotide sequence represented by SEQ ID NO: 166628.

The probes having the nucleotide sequence represented by SEQ ID NOS: 168572, 168573, 168574, 168575, 168576, 168577, 168578, 168579, 168580, 168581, 168582 were selected from the ORFs having the nucleotide sequence represented by SEQ ID NO: 166629.

The probes having the nucleotide sequence represented by SEQ ID NOS: 168583, 168584, 168585, 168586, 168587, 168588, 168589, 168590, 168591, 168592, 168593 were selected from the ORFs having the nucleotide sequence represented by SEQ ID NO: 166630.

The probes having the nucleotide sequence represented by SEQ ID NOS: 168594, 168595, 168596, 168597, 168598, 168599, 168600, 168601, 168602, 168603, 168604 were selected from the ORFs having the nucleotide sequence represented by SEQ ID NO: 166631.

The probes having the nucleotide sequence represented by SEQ ID NOS: 168605, 168606, 168607, 168608, 168609, 168610, 168611, 168612, 168613, 168614, 168615 were selected from the ORFs having the nucleotide sequence represented by SEQ ID NO: 166632.

The probes having the nucleotide sequence represented by SEQ ID NOS: 168616, 168617, 168618, 168619, 168620, 168621, 168622, 168623, 168624, 168625, 168626 were selected from the ORFs having the nucleotide sequence represented by SEQ ID NO: 166633.

The probes having the nucleotide sequence represented by SEQ ID NOS: 168627, 168628, 168629, 168630, 168631, 168632, 168633, 168634, 168635, 168636, 168637 were selected from the ORFs having the nucleotide sequence represented by SEQ ID NO: 166634.

The probes having the nucleotide sequence represented by SEQ ID NOS: 168638, 168639, 168640, 168641, 168642, 168643, 168644, 168645, 168646, 168647, 168648 were selected from the ORFs having the nucleotide sequence represented by SEQ ID NO: 166635.

The probes having the nucleotide sequence represented by SEQ ID NOS: 168649, 168650, 168651, 168652, 168653, 168654, 168655, 168656, 168657, 168658, 168659 were selected from the ORFs having the nucleotide sequence represented by SEQ ID NO: 166636.

The probes having the nucleotide sequence represented by SEQ ID NOS: 168660, 168661, 168662, 168663, 168664, 168665, 168666, 168667, 168668, 168669, 168670 were selected from the ORFs having the nucleotide sequence represented by SEQ ID NO: 166637.

The probes having the nucleotide sequence represented by SEQ ID NOS: 168671, 168672, 168673, 168674, 168675, 168676, 168677, 168678, 168679, 168680, 168681 were selected from the ORFs having the nucleotide sequence represented by SEQ ID NO: 166638.

The probes having the nucleotide sequence represented by SEQ ID NOS: 168682, 168683, 168684, 168685, 168686, 168687, 168688, 168689, 168690, 168691, 168692 were selected from the ORFs having the nucleotide sequence represented by SEQ ID NO: 166639.

The probes having the nucleotide sequence represented by SEQ ID NOS: 168693, 168694, 168695, 168696, 168697, 168698, 168699, 168700, 168701, 168702, 168703 were selected from the ORFs having the nucleotide sequence represented by SEQ ID NO: 166640.

The probes having the nucleotide sequence represented by SEQ ID NOS: 168704, 168705, 168706, 168707, 168708, 168709, 168710, 168711, 168712, 168713, 168714 were selected from the ORFs having the nucleotide sequence represented by SEQ ID NO: 166641.

The probes having the nucleotide sequence represented by SEQ ID NOS: 168715, 168716, 168717, 168718, 168719, 168720, 168721, 168722, 168723, 168724, 168725 were selected from the ORFs having the nucleotide sequence represented by SEQ ID NO: 166642.

The probes having the nucleotide sequence represented by SEQ ID NOS: 168726, 168727, 168728, 168729, 168730, 168731, 168732, 168733, 168734, 168735, 168736 were selected from the ORFs having the nucleotide sequence represented by SEQ ID NO: 166643.

The probes having the nucleotide sequence represented by SEQ ID NOS: 168737, 168738, 168739, 168740, 168741, 168742, 168743, 168744, 168745, 168746, 168747 were selected from the ORFs having the nucleotide sequence represented by SEQ ID NO: 166644.

The probes having the nucleotide sequence represented by SEQ ID NOS: 168748., 168749, 168750, 168751, 168752, 168753, 168754, 168755, 168756, 168757, 168758 were selected from the ORFs having the nucleotide sequence represented by SEQ ID NO: 166645.

The probes having the nucleotide sequence represented by SEQ ID NOS: 168759, 168760, 168761, 168762, 168763, 168764, 168765, 168766, 168767, 168768, 168769 were selected from the ORFs having the nucleotide sequence represented by SEQ ID NO: 166646.

The probes having the nucleotide sequence represented by SEQ ID NOS: 168770, 168771, 168772, 168773, 168774, 168775, 168776, 168777, 168778, 168779, 168780 were selected from the ORFs having the nucleotide sequence represented by SEQ ID NO: 166647.

The probes having the nucleotide sequence represented by SEQ ID NOS: 168781, 168782, 168783, 168784, 168785, 168786, 168787, 168788, 168789, 168790, 168791 were selected from the ORFs having the nucleotide sequence represented by SEQ ID NO: 166648.

The probes having the nucleotide sequence represented by SEQ ID NOS: 168792, 168793, 168794, 168795, 168796, 168797, 168798, 168799, 168800, 168801, 168802 were selected from the ORFs having the nucleotide sequence represented by SEQ ID NO: 166649.

The probes having the nucleotide sequence represented by SEQ ID NOS: 168803, 168804, 168805, 168806, 168807, 168808, 168809, 168810, 168811, 168812, 168813 were selected from the ORFs having the nucleotide sequence represented by SEQ ID NO: 166650.

The probes having the nucleotide sequence represented by SEQ ID NOS: 168814, 168815, 168816, 168817, 168818, 168819, 168820, 168821, 168822, 168823, 168824 were selected from the ORFs having the nucleotide sequence represented by SEQ ID NO: 166651.

The probes having the nucleotide sequence represented by SEQ ID NOS: 168825, 168826, 168827, 168828, 168829, 168830, 168831, 168832, 168833, 168834, 168835 were selected from the ORFs having the nucleotide sequence represented by SEQ ID NO: 166652.

The probes having the nucleotide sequence represented by SEQ ID NOS: 168836, 168837, 168838, 168839, 168840, 168841, 168842, 168843, 168844, 168845, 168846 were selected from the ORFs having the nucleotide sequence represented by SEQ ID NO: 166653.

The probes having the nucleotide sequence represented by SEQ ID NOS: 168847, 168848, 168849, 168850, 168851, 168852, 168853, 168854, 168855, 168856, 168857 were selected from the ORFs having the nucleotide sequence represented by SEQ ID NO: 166654.

The probes having the nucleotide sequence represented by SEQ ID NOS: 168858, 168859, 168860, 168861, 168862, 168863, 168864, 168865, 168866, 168867, 168868 were selected from the ORFs having the nucleotide sequence represented by SEQ ID NO: 166655.

The probes having the nucleotide sequence represented by SEQ ID NOS: 168869, 168870, 168871, 168872, 168873, 168874, 168875, 168876, 168877, 168878, 168879 were selected from the ORFs having the nucleotide sequence represented by SEQ ID NO: 166656.

The probes having the nucleotide sequence represented by SEQ ID NOS: 168880, 168881, 168882, 168883, 168884, 168885, 168886, 168887, 168888, 168889, 168890 were selected from the ORFs having the nucleotide sequence represented by SEQ ID NO: 166657.

The probes having the nucleotide sequence represented by SEQ ID NOS: 168891, 168892, 168893, 168894, 168895, 168896, 168897, 168898, 168899, 168900, 168901 were selected from the ORFs having the nucleotide sequence represented by SEQ ID NO: 166658.

The probes having the nucleotide sequence represented by SEQ ID NOS: 168902, 168903, 168904, 168905, 168906, 168907, 168908, 168909, 168910, 168911, 168912 were selected from the ORFs having the nucleotide sequence represented by SEQ ID NO: 166659.

The probes having the nucleotide sequence represented by SEQ ID NOS: 168913, 168914, 168915, 168916, 168917, 168918, 168919, 168920, 168921, 168922, 168923 were selected from the ORFs having the nucleotide sequence represented by SEQ ID NO: 166660.

The probes having the nucleotide sequence represented by SEQ ID NOS: 168924, 168925, 168926, 168927, 168928, 168929, 168930, 168931, 168932, 168933, 168934 were selected from the ORFs having the nucleotide sequence represented by SEQ ID NO: 166661.

The probes having the nucleotide sequence represented by SEQ ID NOS: 168935, 168936, 168937, 168938, 168939, 168940, 168941, 168942, 168943, 168944, 168945 were selected from the ORFs having the nucleotide sequence represented by SEQ ID NO: 166662.

The probes having the nucleotide sequence represented by SEQ ID NOS: 168946, 168947, 168948, 168949, 168950, 168951, 168952, 168953, 168954, 168955, 168956 were selected from the ORFs having the nucleotide sequence represented by SEQ ID NO: 166663.

The probes having the nucleotide sequence represented by SEQ ID NOS: 168957, 168958, 168959, 168960, 168961, 168962, 168963, 168964, 168965, 168966, 168967 were selected from the ORFs having the nucleotide sequence represented by SEQ ID NO: 166664.

The probes having the nucleotide sequence represented by SEQ ID NOS: 168968, 168969, 168970, 168971, 168972, 168973, 168974, 168975, 168976, 168977, 168978 were selected from the ORFs having the nucleotide sequence represented by SEQ ID NO: 166665.

The probes having the nucleotide sequence represented by SEQ ID NOS: 168979, 168980, 168981, 168982, 168983, 168984, 168985, 168986, 168987, 168988, 168989 were selected from the ORFs having the nucleotide sequence represented by SEQ ID NO: 166666.

The probes having the nucleotide sequence represented by SEQ ID NOS: 168990, 168991, 168992, 168993, 168994, 168995, 168996, 168997, 168998, 168999, 169000 were selected from the ORFs having the nucleotide sequence represented by SEQ ID NO: 166667.

The probes having the nucleotide sequence represented by SEQ ID NOS: 169001, 169002, 169003, 169004, 169005, 169006, 169007, 169008, 169009, 169010, 169011 were selected from the ORFs having the nucleotide sequence represented by SEQ ID NO: 166668.

The probes having the nucleotide sequence represented by SEQ ID NOS: 169012, 169013, 169014, 169015, 169016, 169017, 169018, 169019, 169020, 169021, 169022 were selected from the ORFs having the nucleotide sequence represented by SEQ ID NO: 166669.

The probes having the nucleotide sequence represented by SEQ ID NOS: 169023, 169024, 169025, 169026, 169027, 169028, 169029, 169030, 169031, 169032, 169033 were selected from the ORFs having the nucleotide sequence represented by SEQ ID NO: 166670.

The probes having the nucleotide sequence represented by SEQ ID NOS: 169034, 169035, 169036, 169037, 169038, 169039, 169040, 169041, 169042, 169043, 169044 were selected from the ORFs having the nucleotide sequence represented by SEQ ID NO: 166671.

The probes having the nucleotide sequence represented by SEQ ID NOS: 169045, 169046, 169047, 169048, 169049, 169050, 169051, 169052, 169053, 169054, 169055 were selected from the ORFs having the nucleotide sequence represented by SEQ ID NO: 166672.

The probes having the nucleotide sequence represented by SEQ ID NOS: 169056, 169057, 169058, 169059, 169060, 169061, 169062, 169063, 169064, 169065, 169066 were selected from the ORFs having the nucleotide sequence represented by SEQ ID NO: 166673.

The probes having the nucleotide sequence represented by SEQ ID NOS: 169067, 169068, 169069, 169070, 169071, 169072, 169073, 169074, 169075, 169076, 169077 were selected from the ORFs having the nucleotide sequence represented by SEQ ID NO: 166674.

The probes having the nucleotide sequence represented by SEQ ID NOS: 169078, 169079, 169080, 169081, 169082, 169083, 169084, 169085, 169086, 169087, 169088 were selected from the ORFs having the nucleotide sequence represented by SEQ ID NO: 166675.

The probes having the nucleotide sequence represented by SEQ ID NOS: 169089, 169090, 169091, 169092, 169093, 169094, 169095, 169096, 169097, 169098, 169099 were selected from the ORFs having the nucleotide sequence represented by SEQ ID NO: 166676.

The probes having the nucleotide sequence represented by SEQ ID NOS: 169100, 169101, 169102, 169103, 169104, 169105, 169106, 169107, 169108, 169109, 169110 were selected from the ORFs having the nucleotide sequence represented by SEQ ID NO: 166677.

The probes having the nucleotide sequence represented by SEQ ID NOS: 169111, 169112, 169113, 169114, 169115, 169116, 169117, 169118, 169119, 169120, 169121 were selected from the ORFs having the nucleotide sequence represented by SEQ ID NO: 166678.

The probes having the nucleotide sequence represented by SEQ ID NOS: 169122, 169123, 169124, 169125, 169126, 169127, 169128, 169129, 169130, 169131, 169132 were selected from the ORFs having the nucleotide sequence represented by SEQ ID NO: 166679.

The probes having the nucleotide sequence represented by SEQ ID NOS: 169133, 169134, 169135, 169136, 169137, 169138, 169139, 169140, 169141, 169142, 169143 were selected from the ORFs having the nucleotide sequence represented by SEQ ID NO: 166680.

The probes having the nucleotide sequence represented by SEQ ID NOS: 169144, 169145, 169146, 169147, 169148, 169149, 169150, 169151, 169152, 169153, 169154 were selected from the ORFs having the nucleotide sequence represented by SEQ ID NO: 166681.

The probes having the nucleotide sequence represented by SEQ ID NOS: 169155, 169156, 169157, 169158, 169159, 169160, 169161, 169162, 169163, 169164, 169165 were selected from the ORFs having the nucleotide sequence represented by SEQ ID NO: 166682.

The probes having the nucleotide sequence represented by SEQ ID NOS: 169166, 169167, 169168, 169169, 169170, 169171, 169172, 169173, 169174, 169175, 169176 were selected from the ORFs having the nucleotide sequence represented by SEQ ID NO: 166683.

The probes having the nucleotide sequence represented by SEQ ID NOS: 169177, 169178, 169179, 169180, 169181, 169182, 169183, 169184, 169185, 169186, 169187 were selected from the ORFs having the nucleotide sequence represented by SEQ ID NO: 166684.

The probes having the nucleotide sequence represented by SEQ ID NOS: 169188, 169189, 169190, 169191, 169192, 169193, 169194, 169195, 169196, 169197, 169198 were selected from the ORFs having the nucleotide sequence represented by SEQ ID NO: 166685.

The probes having the nucleotide sequence represented by SEQ ID NOS: 169199, 169200, 169201, 169202, 169203, 169204, 169205, 169206, 169207, 169208, 169209 were selected from the ORFs having the nucleotide sequence represented by SEQ ID NO: 166686.

The probes having the nucleotide sequence represented by SEQ ID NOS: 169210, 169211, 169212, 169213, 169214, 169215, 169216, 169217, 169218, 169219, 169220 were selected from the ORFs having the nucleotide sequence represented by SEQ ID NO: 166687.

The probes having the nucleotide sequence represented by SEQ ID NOS: 169221, 169222, 169223, 169224, 169225, 169226, 169227, 169228, 169229, 169230, 169231 were selected from the ORFs having the nucleotide sequence represented by SEQ ID NO: 166688.

The probes having the nucleotide sequence represented by SEQ ID NOS: 169232, 169233, 169234, 169235, 169236, 169237, 169238, 169239, 169240, 169241, 169242 were selected from the ORFs having the nucleotide sequence represented by SEQ ID NO: 166689.

The probes having the nucleotide sequence represented by SEQ ID NOS: 169243, 169244, 169245, 169246, 169247, 169248, 169249, 169250, 169251, 169252, 169253 were selected from the ORFs having the nucleotide sequence represented by SEQ ID NO: 166690.

The probes having the nucleotide sequence represented by SEQ ID NOS: 169254, 169255, 169256, 169257, 169258, 169259, 169260, 169261, 169262, 169263, 169264 were selected from the ORFs having the nucleotide sequence represented by SEQ ID NO: 166691.

The probes having the nucleotide sequence represented by SEQ ID NOS: 169265, 169266, 169267, 169268, 169269, 169270, 169271, 169272, 169273, 169274, 169275 were selected from the ORFs having the nucleotide sequence represented by SEQ ID NO: 166692.

The probes having the nucleotide sequence represented by SEQ ID NOS: 169276, 169277, 169278, 169279, 169280, 169281, 169282, 169283, 169284, 169285, 169286 were selected from the ORFs having the nucleotide sequence represented by SEQ ID NO: 166693.

The probes having the nucleotide sequence represented by SEQ ID NOS: 169287, 169288, 169289, 169290, 169291, 169292, 169293, 169294, 169295, 169296, 169297 were selected from the ORFs having the nucleotide sequence represented by SEQ ID NO: 166694.

The probes having the nucleotide sequence represented by SEQ ID NOS: 169298, 169299, 169300, 169301, 169302, 169303, 169304, 169305, 169306, 169307, 169308 were selected from the ORFs having the nucleotide sequence represented by SEQ ID NO: 166695.

The probes having the nucleotide sequence represented by SEQ ID NOS: 169309, 169310, 169311, 169312, 169313, 169314, 169315, 169316, 169317, 169318, 169319 were selected from the ORFs having the nucleotide sequence represented by SEQ ID NO: 166696.

The probes having the nucleotide sequence represented by SEQ ID NOS: 169320, 169321, 169322, 169323, 169324, 169325, 169326, 169327, 169328, 169329, 169330 were selected from the ORFs having the nucleotide sequence represented by SEQ ID NO: 166697.

The probes having the nucleotide sequence represented by SEQ ID NOS: 169331, 169332, 169333, 169334, 169335, 169336, 169337, 169338, 169339, 169340, 169341 were selected from the ORFs having the nucleotide sequence represented by SEQ ID NO: 166698.

The probes having the nucleotide sequence represented by SEQ ID NOS: 169342, 169343, 169344, 169345, 169346, 169347, 169348, 169349, 169350, 169351, 169352 were selected from the ORFs having the nucleotide sequence represented by SEQ ID NO: 166699.

The probes having the nucleotide sequence represented by SEQ ID NOS: 169353, 169354, 169355, 169356, 169357, 169358, 169359, 169360, 169361, 169362, 169363 were selected from the ORFs having the nucleotide sequence represented by SEQ ID NO: 166700.

The probes having the nucleotide sequence represented by SEQ ID NOS: 169364, 169365, 169.366, 169367, 169368, 169369, 169370, 169371, 169372, 169373, 169374 were selected from the ORFs having the nucleotide sequence represented by SEQ ID NO: 166701.

The probes having the nucleotide sequence represented by SEQ ID NOS: 169375, 169376, 169377, 169378, 169379, 169380, 169381, 169382, 169383, 169384, 169385 were selected from the ORFs having the nucleotide sequence represented by SEQ ID NO: 166702.

The probes having the nucleotide sequence represented by SEQ ID NOS: 169386, 169387, 169388, 169389, 169390, 169391, 169392, 169393, 169394, 169395, 169396 were selected from the ORFs having the nucleotide sequence represented by SEQ ID NO: 166703.

The probes having the nucleotide sequence represented by SEQ ID NOS: 169397, 169398, 169399, 169400, 169401, 169402, 169403, 169404, 169405, 169406, 169407 were selected from the ORFs having the nucleotide sequence represented by SEQ ID NO: 166704.

The probes having the nucleotide sequence represented by SEQ ID NOS: 169408, 169409, 169410, 169411, 169412, 169413, 169414, 169415, 169416, 169417, 169418 were selected from the ORFs having the nucleotide sequence represented by SEQ ID NO: 166705.

The probes having the nucleotide sequence represented by SEQ ID NOS: 169419, 169420, 169421, 169422, 169423, 169424, 169425, 169426, 169427, 169428, 169429 were selected from the ORFs having the nucleotide sequence represented by SEQ ID NO: 166706.

The probes having the nucleotide sequence represented by SEQ ID NOS: 169430, 169431, 169432, 169433, 169434, 169435, 169436, 169437, 169438, 169439, 169440 were selected from the ORFs having the nucleotide sequence represented by SEQ ID NO: 166707.

The probes having the nucleotide sequence represented by SEQ ID NOS: 169441, 169442, 169443, 169444, 169445, 169446, 169447, 169448, 169449, 169450, 169451 were selected from the ORFs having the nucleotide sequence represented by SEQ ID NO: 166708.

The probes having the nucleotide sequence represented by SEQ ID NOS: 169452, 169453, 169454, 169455, 169456, 169457, 169458, 169459, 169460, 169461, 169462 were selected from the ORFs having the nucleotide sequence represented by SEQ ID NO: 166709.

The probes having the nucleotide sequence represented by SEQ ID NOS: 169463, 169464, 169465, 169466, 169467, 169468, 169469, 169470, 169471, 169472, 169473 were selected from the ORFs having the nucleotide sequence represented by SEQ ID NO: 166710.

The probes having the nucleotide sequence represented by SEQ ID NOS: 169474, 169475, 169476, 169477, 169478, 169479, 169480, 169481, 169482, 169483, 169484 were selected from the ORFs having the nucleotide sequence represented by SEQ ID NO: 166711.

The probes having the nucleotide sequence represented by SEQ ID NOS: 169485, 169486, 169487, 169488, 169489, 169490, 169491, 169492, 169493, 169494, 169495 were selected from the ORFs having the nucleotide sequence represented by SEQ ID NO: 166712.

The probes having the nucleotide sequence represented by SEQ ID NOS: 169496, 169497, 169498, 169499, 169500, 169501, 169502, 169503, 169504, 169505, 169506 were selected from the ORFs having the nucleotide sequence represented by SEQ ID NO: 166713.

The probes having the nucleotide sequence represented by SEQ ID NOS: 169507, 169508, 169509, 169510, 169511, 169512, 169513, 169514, 169515, 169516, 169517 were selected from the ORFs having the nucleotide sequence represented by SEQ ID NO: 166714.

The probes having the nucleotide sequence represented by SEQ ID NOS: 169518, 169519, 169520, 169521, 169522, 169523, 169524, 169525, 169526, 169527, 169528 were selected from the ORFs having the nucleotide sequence represented by SEQ ID NO: 166715.

The probes having the nucleotide sequence represented by SEQ ID NOS: 169529., 169530, 169531, 169532, 169533, 169534, 169535, 169536, 169537, 169538, 169539 were selected from the ORFs having the nucleotide sequence represented by SEQ ID NO: 166716.

The probes having the nucleotide sequence represented by SEQ ID NOS: 169540, 169541, 169542, 169543, 169544, 169545, 169546, 169547, 169548, 169549, 169550 were selected from the ORFs having the nucleotide sequence represented by SEQ ID NO: 166717.

The probes having the nucleotide sequence represented by SEQ ID NOS: 169551, 169552, 169553, 169554, 169555, 169556, 169557, 169558, 169559, 169560, 169561 were selected from the ORFs having the nucleotide sequence represented by SEQ ID NO: 166718.

The probes having the nucleotide sequence represented by SEQ ID NOS: 169562, 169563, 169564, 169565, 169566, 169567, 169568, 169569, 169570, 169571, 169572 were selected from the ORFs having the nucleotide sequence represented by SEQ ID NO: 166719.

The probes having the nucleotide sequence represented by SEQ ID NOS: 169573, 169574, 169575, 169576, 169577, 169578, 169579, 169580, 169581, 169582, 169583 were selected from the ORFs having the nucleotide sequence represented by SEQ ID NO: 166720.

The probes having the nucleotide sequence represented by SEQ ID NOS: 169584, 169585, 169586, 169587, 169588, 169589, 169590, 169591, 169592, 169593, 169594 were selected from the ORFs having the nucleotide sequence represented by SEQ ID NO: 166721.

The probes having the nucleotide sequence represented by SEQ ID NOS: 169595, 169596, 169597, 169598, 169599, 169600, 169601, 169602, 169603, 169604, 169605 were selected from the ORFs having the nucleotide sequence represented by SEQ ID NO: 166722.

The probes having the nucleotide sequence represented by SEQ ID NOS: 169606, 169607, 169608, 169609, 169610, 169611, 169612, 169613, 169614, 169615, 169616 were selected from the ORFs having the nucleotide sequence represented by SEQ ID NO: 166723.

The probes having the nucleotide sequence represented by SEQ ID NOS: 169617, 169618, 169619, 169620, 169621, 169622, 169623, 169624, 169625, 169626, 169627 were selected from the ORFs having the nucleotide sequence represented by SEQ ID NO: 166724.

The probes having the nucleotide sequence represented by SEQ ID NOS: 169628, 169629, 169630, 169631, 169632, 169633, 169634, 169635, 169636, 169637, 169638 were selected from the ORFs having the nucleotide sequence represented by SEQ ID NO: 166725.

The probes having the nucleotide sequence represented by SEQ ID NOS: 169639, 169640, 169641, 169642, 169643, 169644, 169645, 169646, 169647, 169648, 169649 were selected from the ORFs having the nucleotide sequence represented by SEQ ID NO: 166726.

The probes having the nucleotide sequence represented by SEQ ID NOS: 169650, 169651, 169652, 169653, 169654, 169655, 169656, 169657, 169658, 169659, 169660 were selected from the ORFs having the nucleotide sequence represented by SEQ ID NO: 166727.

The probes having the nucleotide sequence represented by SEQ ID NOS: 169661, 169662, 169663, 169664, 169665, 169666, 169667, 169668, 169669, 169670, 169671 were selected from the ORFs having the nucleotide sequence represented by SEQ ID NO: 166728.

The probes having the nucleotide sequence represented by SEQ ID NOS: 169672, 169673, 169674, 169675, 169676, 169677, 169678, 169679, 169680, 169681, 169682 were selected from the ORFs having the nucleotide sequence represented by SEQ ID NO: 166729.

The probes having the nucleotide sequence represented by SEQ ID NOS: 169683, 169684, 169685, 169686, 169687, 169688, 169689, 169690, 169691, 169692, 169693 were selected from the ORFs having the nucleotide sequence represented by SEQ ID NO: 166730.

The probes having the nucleotide sequence represented by SEQ ID NOS: 169694, 169695, 169696, 169697, 169698, 169699, 169700, 169701, 169702, 169703, 169704 were selected from the ORFs having the nucleotide sequence represented by SEQ ID NO: 166731.

The probes having the nucleotide sequence represented by SEQ ID NOS: 169705, 169706, 169707, 169708, 169709, 169710, 169711, 169712, 169713, 169714, 169715 were selected from the ORFs having the nucleotide sequence represented by SEQ ID NO: 166732.

The probes having the nucleotide sequence represented by SEQ ID NOS: 169716, 169717, 169718, 169719, 169720, 169721, 169722, 169723, 169724, 169725, 169726 were selected from the ORFs having the nucleotide sequence represented by SEQ ID NO: 166733.

The probes having the nucleotide sequence represented by SEQ ID NOS: 169727, 169728, 169729, 169730, 169731, 169732, 169733, 169734, 169735, 169736, 169737 were selected from the ORFs having the nucleotide sequence represented by SEQ ID NO: 166734.

The probes having the nucleotide sequence represented by SEQ ID NOS: 169738, 169739, 169740, 169741, 169742, 169743, 169744, 169745, 169746, 169747, 169748 were selected from the ORFs having the nucleotide sequence represented by SEQ ID NO: 166735.

The probes having the nucleotide sequence represented by SEQ ID NOS: 169749, 169750, 169751, 169752, 169753, 169754, 169755, 169756, 169757, 169758, 169759 were selected from the ORFs having the nucleotide sequence represented by SEQ ID NO: 166736.

The probes having the nucleotide sequence represented by SEQ. ID NOS: 169760, 169761, 169762, 169763, 169764, 169765, 169766, 169767, 169768, 169769, 169770 were selected from the ORFs having the nucleotide sequence represented by SEQ ID NO: 166737.

The probes having the nucleotide sequence represented by SEQ ID NOS: 169771, 169772, 169773, 169774, 169775, 169776, 169777, 169778, 169779, 169780, 169781 were selected from the ORFs having the nucleotide sequence represented by SEQ ID NO: 166738.

The probes having the nucleotide sequence represented by SEQ ID NOS: 169782, 169783, 169784, 169785, 169786, 169787, 169788, 169789, 169790, 169791, 169792 were selected from the ORFs having the nucleotide sequence represented by SEQ ID NO: 166739.

The probes having the nucleotide sequence represented by SEQ ID NOS: 169793, 169794, 169795, 169796, 169797, 169798, 169799, 169800, 169801, 169802, 169803 were selected from the ORFs having the nucleotide sequence represented by SEQ ID NO: 166740.

The probes having the nucleotide sequence represented by SEQ ID NOS: 169804, 169805, 169806, 169807, 169808, 169809, 169810, 169811, 169812, 169813, 169814 were selected from the ORFs having the nucleotide sequence represented by SEQ ID NO: 166741.

The probes having the nucleotide sequence represented by SEQ ID NOS: 169815, 169816, 169817, 169818, 169819, 169820, 169821, 169822, 169823, 169824, 169825 were selected from the ORFs having the nucleotide sequence represented by SEQ ID NO: 166742.

The probes having the nucleotide sequence represented by SEQ ID NOS: 169826, 169827, 169828, 169829, 169830, 169831, 169832, 169833, 169834, 169835, 169836 were selected from the ORFs having the nucleotide sequence represented by SEQ ID NO: 166743.

The probes having the nucleotide sequence represented by SEQ ID NOS: 169837, 169838, 169839, 169840, 169841, 169842, 169843, 169844, 169845, 169846, 169847 were selected from the ORFs having the nucleotide sequence represented by SEQ ID NO: 166744.

The probes having the nucleotide sequence represented by SEQ ID NOS: 169848, 169849, 169850, 169851, 169852, 169853, 169854, 169855, 169856, 169857, 169858 were selected from the ORFs having the nucleotide sequence represented by SEQ ID NO: 166745.

The probes having the nucleotide sequence represented by SEQ ID NOS: 169859, 169860, 169861, 169862, 169863, 169864, 169865, 169866, 169867, 169868, 169869 were selected from the ORFs having the nucleotide sequence represented by SEQ ID NO: 166746.

The probes having the nucleotide sequence represented by SEQ ID NOS: 169870, 169871, 169872, 169873, 169874, 169875, 169876, 169877, 169878, 169879, 169880 were selected from the ORFs having the nucleotide sequence represented by SEQ ID NO: 166747.

The probes having the nucleotide sequence represented by SEQ ID NOS: 169881, 169882, 169883, 169884, 169885, 169886, 169887, 169888, 169889, 169890, 169891 were selected from the ORFs having the nucleotide sequence represented by SEQ ID NO: 166748.

The probes having the nucleotide sequence represented by SEQ ID NOS: 169892, 169893, 169894, 169895, 169896, 169897, 169898, 169899, 169900, 169901, 169902 were selected from the ORFs having the nucleotide sequence represented by SEQ ID NO: 166749.

The probes having the nucleotide sequence represented by SEQ ID NOS: 169903, 169904, 169905, 169906, 169907, 169908, 169909, 169910, 169911, 169912, 169913 were selected from the ORFs having the nucleotide sequence represented by SEQ ID NO: 166750.

The probes having the nucleotide sequence represented by SEQ ID NOS: 169914, 169915, 169916, 169917, 169918, 169919, 169920, 169921, 169922, 169923, 169924 were selected from the ORFs having the nucleotide sequence represented by SEQ ID NO: 166751.

The probes having the nucleotide sequence represented by SEQ ID NOS: 169925, 169926, 169927, 169928, 169929, 169930, 169931, 169932, 169933, 169934, 169935 were selected from the ORFs having the nucleotide sequence represented by SEQ ID NO: 166752.

The probes having the nucleotide sequence represented by SEQ ID NOS: 169936, 169937, 169938, 169939, 169940, 169941, 169942, 169943, 169944, 169945, 169946 were selected from the ORFs having the nucleotide sequence represented by SEQ ID NO: 166753.

probes having the nucleotide sequence represented by SEQ ID NOS: 169947, 169948, 169949, 169950, 169951, 169952, 169953, 169954, 169955, 169956, 169957 were selected from the ORFs having the nucleotide sequence represented by SEQ ID NO: 166754.

The probes having the nucleotide sequence represented by SEQ ID NOS: 169958, 169959, 169960, 169961, 169962, 169963, 169964, 169965, 169966, 169967, 169968 were selected from the ORFs having the nucleotide sequence represented by SEQ ID NO: 166755.

The probes having the nucleotide sequence represented by SEQ ID NOS: 169969, 169970, 169971, 169972, 169973, 169974, 169975, 169976, 169977, 169978, 169979 were selected from the ORFs having the nucleotide sequence represented by SEQ ID NO: 166756.

The probes having the nucleotide sequence represented by SEQ ID NOS: 169980, 169981, 169982, 169983, 169984, 169985, 169986, 169987, 169988, 169989, 169990 were selected from the ORFs having the nucleotide sequence represented by SEQ ID NO: 166757.

The probes having the nucleotide sequence represented by SEQ ID NOS: 169991, 169992, 169993, 169994, 169995, 169996, 169997, 169998, 169999, 170000, 170001 were selected from the ORFs having the nucleotide sequence represented by SEQ ID NO: 166758.

The probes having the nucleotide sequence represented by SEQ ID NOS: 170002, 170003, 170004, 170005, 170006, 170007, 170008, 170009, 170010, 170011, 170012 were selected from the ORFs having the nucleotide sequence represented by SEQ ID NO: 166759.

The probes having the nucleotide sequence represented by SEQ ID NOS: 170013, 170014, 170015, 170016, 170017, 170018, 170019, 170020, 170021, 170022, 170023 were selected from the ORFs having the nucleotide sequence represented by SEQ ID NO: 166760.

The probes having the nucleotide sequence represented by SEQ ID NOS: 170024, 170025, 170026, 170027, 170028, 170029, 170030, 170031, 170032, 170033, 170034 were selected from the ORFs having the nucleotide sequence represented by SEQ ID NO: 166761.

The probes having the nucleotide sequence represented by SEQ ID NOS: 170035, 170036, 170037, 170038, 170039, 170040, 170041, 170042, 170043, 170044, 170045 were selected from the ORFs having the nucleotide sequence represented by SEQ ID NO: 166762.

The probes having the nucleotide sequence represented by SEQ ID NOS: 170046, 170047, 170048, 170049, 170050, 170051, 170052, 170053, 170054, 170055, 170056 were selected from the ORFs having the nucleotide sequence represented by SEQ ID NO: 166763.

The probes having the nucleotide sequence represented by SEQ ID NOS: 170057, 170058, 170059, 170060, 170061, 170062, 170063, 170064, 170065, 170066, 170067 were selected from the ORFs having the nucleotide sequence represented by SEQ ID NO: 166764.

The probes having the nucleotide sequence represented by SEQ ID NOS: 170068, 170069, 170070, 170071, 170072, 170073, 170074, 170075, 170076, 170077, 170078 were selected from the ORFs having the nucleotide sequence represented by SEQ ID NO: 166765.

The probes having the nucleotide sequence represented by SEQ ID NOS: 170079, 170080, 170081, 170082, 170083, 170084, 170085, 170086, 170087, 170088, 170089 were selected from the ORFs having the nucleotide sequence represented by SEQ ID NO: 166766.

The probes having the nucleotide sequence represented by SEQ ID NOS: 170090, 170091, 170092, 170093, 170094, 170095, 170096, 170097, 170098, 170099, 170100 were selected from the ORFs having the nucleotide sequence represented by SEQ ID NO: 166767.

The probes having the nucleotide sequence represented by SEQ ID NOS: 170101, 170102, 170103, 170104, 170105, 170106, 170107, 170108, 170109, 170110, 170111 were selected from the ORFs having the nucleotide sequence represented by SEQ ID NO: 166768.

The probes having the nucleotide sequence represented by SEQ ID NOS: 170112, 170113, 170114, 170115, 170116, 170117, 170118, 170119, 170120, 170121, 170122 were selected from the ORFs having the nucleotide sequence represented by SEQ ID NO: 166769.

The probes having the nucleotide sequence represented by SEQ ID NOS: 170123, 170124, 170125, 170126, 170127, 170128, 170129, 170130, 170131, 170132, 170133 were selected from the ORFs having the nucleotide sequence represented by SEQ ID NO: 166770.

The probes having the nucleotide sequence represented by SEQ ID NOS: 170134, 170135, 170136, 170137, 170138, 170139, 170140, 170141, 170142, 170143, 170144 were selected from the ORFs having the nucleotide sequence represented by SEQ ID NO: 166771.

The probes having the nucleotide sequence represented by SEQ ID NOS: 170145, 170146, 170147, 170148, 170149, 170150, 170151, 170152, 170153, 170154, 170155 were selected from the ORFs having the nucleotide sequence represented by SEQ ID NO: 166772.

The probes having the nucleotide sequence represented by SEQ ID NOS: 170156, 170157, 170158, 170159, 170160, 170161, 170162, 170163, 170164, 170165, 170166 were selected from the ORFs having the nucleotide sequence represented by SEQ ID NO: 166773.

The probes having the nucleotide sequence represented by SEQ ID NOS: 170167, 170168, 170169, 170170, 170171, 170172, 170173, 170174, 170175, 170176, 170177 were selected from the ORFs having the nucleotide sequence represented by SEQ ID NO: 166774.

The probes having the nucleotide sequence represented by SEQ ID NOS: 170178, 170179, 170180, 170181, 170182, 170183, 170184, 170185, 170186, 170187, 170188 were selected from the ORFs having the nucleotide sequence represented by SEQ ID NO: 166775.

The probes having the nucleotide sequence represented by SEQ ID NOS: 170189, 170190, 170191, 170192, 170193, 170194, 170195, 170196, 170197, 170198, 170199 were selected from the ORFs having the nucleotide sequence represented by SEQ ID NO: 166776.

The probes having the nucleotide sequence represented by SEQ ID NOS: 170200, 170201, 170202, 170203, 170204, 170205, 170206, 170207, 170208, 170209, 170210 were selected from the ORFs having the nucleotide sequence represented by SEQ ID NO: 166777.

The probes having the nucleotide sequence represented by SEQ ID NOS: 170211, 170212, 170213, 170214, 170215, 170216, 170217, 170218, 170219, 170220, 170221 were selected from the ORFs having the nucleotide sequence represented by SEQ ID NO: 166778.

The probes having the nucleotide sequence represented by SEQ ID NOS: 170222, 170223, 170224, 170225, 170226, 170227, 170228, 170229, 170230, 170231, 170232 were selected from the ORFs having the nucleotide sequence represented by SEQ ID NO: 166779.

The probes having the nucleotide sequence represented by SEQ ID NOS: 170233, 170234, 170235, 170236, 170237, 170238, 170239, 170240, 170241, 170242, 170243 were selected from the ORFs having the nucleotide sequence represented by SEQ ID NO: 166780.

The probes having the nucleotide sequence represented by SEQ ID NOS: 170244, 170245, 170246, 170247, 170248, 170249, 170250, 170251, 170252, 170253, 170254 were selected from the ORFs having the nucleotide sequence represented by SEQ ID NO: 166781.

The probes having the nucleotide sequence represented by SEQ ID NOS: 170255, 170256, 170257, 170258, 170259, 170260, 170261, 170262, 170263, 170264, 170265 were selected from the ORFs having the nucleotide sequence represented by SEQ ID NO: 166782.

The probes having the nucleotide sequence represented by SEQ ID NOS: 170266, 170267, 170268, 170269, 170270, 170271, 170272, 170273, 170274, 170275, 170276 were selected from the ORFs having the nucleotide sequence represented by SEQ ID NO: 166783.

The probes having the nucleotide sequence represented by SEQ ID NOS: 170277, 170278, 170279, 170280, 170281, 170282, 170283, 170284, 170285, 170286, 170287 were selected from the ORFs having the nucleotide sequence represented by SEQ ID NO: 166784.

The probes having the nucleotide sequence represented by SEQ ID NOS: 170288, 170289, 170290, 170291, 170292, 170293, 170294, 170295, 170296, 170297, 170298 were selected from the ORFs having the nucleotide sequence represented by SEQ ID NO: 166785.

The probes having the nucleotide sequence represented by SEQ ID NOS: 170299, 170300, 170301, 170302, 170303, 170304, 170305, 170306, 170307, 170308, 170309 were selected from the ORFs having the nucleotide sequence represented by SEQ ID NO: 166786.

The probes having the nucleotide sequence represented by SEQ ID NOS: 170310, 170311, 170312, 170313, 170314, 170315, 170316, 170317, 170318, 170319, 170320 were selected from the ORFs having the nucleotide sequence represented by SEQ ID NO: 166787.

The probes having the nucleotide sequence represented by SEQ ID NOS: 170321, 170322, 170323, 170324, 170325, 170326, 170327, 170328, 170329, 170330, 170331 were selected from the ORFs having the nucleotide sequence represented by SEQ ID NO: 166788.

The probes having the nucleotide sequence represented by SEQ ID NOS: 170332, 170333, 170334, 170335, 170336, 170337, 170338, 170339, 170340, 170341, 170342 were selected from the ORFs having the nucleotide sequence represented by SEQ ID NO: 166789.

The probes having the nucleotide sequence represented by SEQ ID NOS: 170343, 170344, 170345, 170346, 170347, 170348, 170349, 170350, 170351, 170352, 170353 were selected from the ORFs having the nucleotide sequence represented by SEQ ID NO: 166790.

The probes having the nucleotide sequence represented by SEQ ID NOS: 170354, 170355, 170356, 170357, 170358, 170359, 170360, 170361, 170362, 170363, 170364 were selected from the ORFs having the nucleotide sequence represented by SEQ ID NO: 166791.

The probes having the nucleotide sequence represented by SEQ ID NOS: 170365, 170366, 170367, 170368, 170369, 170370, 170371, 170372, 170373, 170374, 170375 were selected from the ORFs having the nucleotide sequence represented by SEQ ID NO: 166792.

The probes having the nucleotide sequence represented by SEQ ID NOS: 170376, 170377, 170378, 170379, 170380, 170381, 170382, 170383, 170384, 170385, 170386 were selected from the ORFs having the nucleotide sequence represented by SEQ ID NO: 166793.

The probes having the nucleotide sequence represented by SEQ ID NOS: 170387, 170388, 170389, 170390, 170391, 170392, 170393, 170394, 170395, 170396, 170397 were selected from the ORFs having the nucleotide sequence represented by SEQ ID NO: 166794.

The probes having the nucleotide sequence represented by SEQ ID NOS: 170398, 170399, 170400, 170401, 170402, 170403, 170404, 170405, 170406, 170407, 170408 were selected from the ORFs having the nucleotide sequence represented by SEQ ID NO: 166795.

The probes having the nucleotide sequence represented by SEQ ID NOS: 170409, 170410, 170411, 170412, 170413, 170414, 170415, 170416, 170417, 170418, 170419 were selected from the ORFs having the nucleotide sequence represented by SEQ ID NO: 166796.

The probes having the nucleotide sequence represented by SEQ ID NOS: 170420, 170421, 170422, 170423, 170424, 170425, 170426, 170427, 170428, 170429, 170430 were selected from the ORFs having the nucleotide sequence represented by SEQ ID NO: 166797.

The probes having the nucleotide sequence represented by SEQ ID NOS: 170431, 170432, 170433, 170434, 170435, 170436, 170437, 170438, 170439, 170440, 170441 were selected from the ORFs having the nucleotide sequence represented by SEQ ID NO: 166798.

The probes having the nucleotide sequence represented by SEQ ID NOS: 170442, 170443, 170444, 170445, 170446, 170447, 170448, 170449, 170450, 170451, 170452 were selected from the ORFs having the nucleotide sequence represented by SEQ ID NO: 166799.

The probes having the nucleotide sequence represented by SEQ ID NOS: 170453, 170454, 170455, 170456, 170457, 170458, 170459, 170460, 170461, 170462, 170463 were selected from the ORFs having the nucleotide sequence represented by SEQ ID NO: 166800.

The probes having the nucleotide sequence represented by SEQ ID NOS: 170464, 170465, 170466, 170467, 170468, 170469, 170470, 170471, 170472, 170473, 170474 were selected from the ORFs having the nucleotide sequence represented by SEQ ID NO: 166801.

The probes having the nucleotide sequence represented by SEQ ID NOS: 170475, 170476, 170477, 170478, 170479, 170480, 170481, 170482, 170483, 170484, 170485 were selected from the ORFs having the nucleotide sequence represented by SEQ ID NO: 166802.

The probes having the nucleotide sequence represented by SEQ ID NOS: 170486, 170487, 170488, 170489, 170490, 170491, 170492, 170493, 170494, 170495, 170496 were selected from the ORFs having the nucleotide sequence represented by SEQ ID NO: 166803.

The probes having the nucleotide sequence represented by SEQ ID NOS: 170497, 170498, 170499, 170500, 170501, 170502, 170503, 170504, 170505, 170506, 170507 were selected from the ORFs having the nucleotide sequence represented by SEQ ID NO: 166804.

The probes having the nucleotide sequence represented by SEQ ID NOS: 170508, 170509, 170510, 170511, 170512, 170513, 170514, 170515, 170516, 170517, 170518 were selected from the ORFs having the nucleotide sequence represented by SEQ ID NO: 166805.

The probes having the nucleotide sequence represented by SEQ ID NOS: 170519, 170520, 170521, 170522, 170523, 170524, 170525, 170526, 170527, 170528, 170529 were selected from the ORFs having the nucleotide sequence represented by SEQ ID NO: 166806.

The probes having the nucleotide sequence represented by SEQ ID NOS: 170530, 170531, 170532, 170533, 170534, 170535, 170536, 170537, 170538, 170539, 170540 were selected from the ORFs having the nucleotide sequence represented by SEQ ID NO: 166807.

The probes having the nucleotide sequence represented by SEQ ID NOS: 170541, 170542, 170543, 170544, 170545, 170546, 170547, 170548, 170549, 170550, 170551 were selected from the ORFs having the nucleotide sequence represented by SEQ ID NO: 166808.

The probes having the nucleotide sequence represented by SEQ ID NOS: 170552, 170553, 170554, 170555, 170556, 170557, 170558, 170559, 170560, 170561, 170562 were selected from the ORFs having the nucleotide sequence represented by SEQ ID NO: 166809.

The probes having the nucleotide sequence represented by SEQ ID NOS: 170563, 170564, 170565, 170566, 170567, 170568, 170569, 170570, 170571, 170572, 170573 were selected from the ORFs having the nucleotide sequence represented by SEQ ID NO: 166810.

The probes having the nucleotide sequence represented by SEQ ID NOS: 170574, 170575, 170576, 170577, 170578, 170579, 170580, 170581, 170582, 170583, 170584 were selected from the ORFs having the nucleotide sequence represented by SEQ ID NO: 166811.

The probes having the nucleotide sequence represented by SEQ ID NOS: 170585, 170586, 170587, 170588, 170589, 170590, 170591, 170592, 170593, 170594, 170595 were selected from the ORFs having the nucleotide sequence represented by SEQ ID NO: 166812.

The probes having the nucleotide sequence represented by SEQ ID NOS: 170596, 170597, 170598, 170599, 170600, 170601, 170602, 170603, 170604, 170605, 170606 were selected from the ORFs having the nucleotide sequence represented by SEQ ID NO: 166813.

The probes having the nucleotide sequence represented by SEQ ID NOS: 170607, 170608, 170609, 170610, 170611, 170612, 170613, 170614, 170615, 170616, 170617 were selected from the ORFs having the nucleotide sequence represented by SEQ ID NO: 166814.

The probes having the nucleotide sequence represented by SEQ ID NOS: 170618, 170619, 170620, 170621, 170622, 170623, 170624, 170625, 170626, 170627, 170628 were selected from the ORFs having the nucleotide sequence represented by SEQ ID NO: 166815.

The probes having the nucleotide sequence represented by SEQ ID NOS: 170629, 170630, 170631, 170632, 170633, 170634, 170635, 170636, 170637, 170638, 170639 were selected from the ORFs having the nucleotide sequence represented by SEQ ID NO: 166816.

The probes having the nucleotide sequence represented by SEQ ID NOS: 170640, 170641, 170642, 170643, 170644, 170645, 170646, 170647, 170648, 170649, 170650 were selected from the ORFs having the nucleotide sequence represented by SEQ ID NO: 166817.

The probes having the nucleotide sequence represented by SEQ ID NOS: 1706.51, 170652, 170653, 170654, 170655, 170656, 170657, 170658, 170659, 170660, 170661 were selected from the ORFs having the nucleotide sequence represented by SEQ ID NO: 166818.

The probes having the nucleotide sequence represented by SEQ ID NOS: 170662, 170663, 170664, 170665, 170666, 170667, 170668, 170669, 170670, 170671, 170672 were selected from the ORFs having the nucleotide sequence represented by SEQ ID NO: 166819.

The probes having the nucleotide sequence represented by SEQ ID NOS: 170673, 170674, 170675, 170676, 170677, 170678, 170679, 170680, 170681, 170682, 170683 were selected from the ORFs having the nucleotide sequence represented by SEQ ID NO: 166820.

The probes having the nucleotide sequence represented by SEQ ID NOS: 170684, 170685, 170686, 170687, 170688, 170689, 170690, 170691, 170692, 170693, 170694 were selected from the ORFs having the nucleotide sequence represented by SEQ ID NO: 166821.

The probes having the nucleotide sequence represented by SEQ ID NOS: 170695, 170696, 170697, 170698, 170699, 170700, 170701, 170702, 170703, 170704, 170705 were selected from the ORFs having the nucleotide sequence represented by SEQ ID NO: 166822.

The probes having the nucleotide sequence represented by SEQ ID NOS: 170706, 170707, 170708, 170709, 170710, 170711, 170712, 170713, 170714, 170715, 170716 were selected from the ORFs having the nucleotide sequence represented by SEQ ID NO: 166823.

The probes having the nucleotide sequence represented by SEQ ID NOS: 170717, 170718, 170719, 170720, 170721, 170722, 170723, 170724, 170725, 170726, 170727 were selected from the ORFs having the nucleotide sequence represented by SEQ ID NO: 166824.

The probes having the nucleotide sequence represented by SEQ ID NOS: 170728, 170729, 170730, 170731, 170732, 170733, 170734, 170735, 170736, 170737, 170738 were selected from the ORFs having the nucleotide sequence represented by SEQ ID NO: 166825.

The probes having the nucleotide sequence represented by SEQ ID NOS: 170739, 170740, 170741, 170742, 170743, 170744, 170745, 170746, 170747, 170748, 170749 were selected from the ORFs having the nucleotide sequence represented by SEQ ID NO: 166826.

The probes having the nucleotide sequence represented by SEQ ID NOS: 170750, 170751, 170752, 170753, 170754, 170755, 170756, 170757, 170758, 170759, 170760 were selected from the ORFs having the nucleotide sequence represented by SEQ ID NO: 166827.

The probes having the nucleotide sequence represented by SEQ ID NOS: 170761, 170762, 170763, 170764, 170765, 170766, 170767, 170768, 170769, 170770, 170771 were selected from the ORFs having the nucleotide sequence represented by SEQ ID NO: 166828.

The probes having the nucleotide sequence represented by SEQ ID NOS: 170772, 170773, 170774, 170775, 170776, 170777, 170778, 170779, 170780, 170781, 170782 were selected from the ORFs having the nucleotide sequence represented by SEQ ID NO: 166829.

The probes having the nucleotide sequence represented by SEQ ID NOS: 170783, 170784, 170785, 170786, 170787, 170788, 170789, 170790, 170791, 170792, 170793 were selected from the ORFs having the nucleotide sequence represented by SEQ ID NO: 166830.

The probes having the nucleotide sequence represented by SEQ ID NOS: 170794, 170795, 170796, 170797, 170798, 170799, 170800, 170801, 170802, 170803, 170804 were selected from the ORFs having the nucleotide sequence represented by SEQ ID NO: 166831.

The probes having the nucleotide sequence represented by SEQ ID NOS: 170805, 170806, 170807, 170808, 170809, 170810, 170811, 170812, 170813, 170814, 170815 were selected from the ORFs having the nucleotide sequence represented by SEQ ID NO: 166832.

The probes having the nucleotide sequence represented by SEQ ID NOS: 170816, 170817, 170818, 170819, 170820, 170821, 170822, 170823, 170824, 170825, 170826 were selected from the ORFs having the nucleotide sequence represented by SEQ ID NO: 166833.

The probes having the nucleotide sequence represented by SEQ ID NOS: 170827, 170828, 170829, 170830, 170831, 170832, 170833, 170834, 170835, 170836, 170837 were selected from the ORFs having the nucleotide sequence represented by SEQ ID NO: 166834.

The probes having the nucleotide sequence represented by SEQ ID NOS: 170838, 170839, 170840, 170841, 170842, 170843, 170844, 170845, 170846, 170847, 170848 were selected from the ORFs having the nucleotide sequence represented by SEQ ID NO: 166835.

The probes having the nucleotide sequence represented by SEQ ID NOS: 170849, 170850, 170851, 170852, 170853, 170854, 170855, 170856, 170857, 170858, 170859 were selected from the ORFs having the nucleotide sequence represented by SEQ ID NO: 166836.

The probes having the nucleotide sequence represented by SEQ ID NOS: 170860, 170861, 170862, 170863, 170864, 170865, 170866, 170867, 170868, 170869, 1i0870 were selected from the ORFs having the nucleotide sequence represented by SEQ ID NO: 166837.

The probes having the nucleotide sequence represented by SEQ ID NOS: 170871, 170872, 170873, 170874, 170875, 170876, 170877, 170878, 170879, 170880, 170881 were selected from the ORFs having the nucleotide sequence represented by SEQ ID NO: 166838.

The probes having the nucleotide sequence represented by SEQ ID NOS: 170882, 170883, 170884, 170885, 170886, 170887, 170888, 170889, 170890, 170891, 170892 were selected from the ORFs having the nucleotide sequence represented by SEQ ID NO: 166839.

The probes having the nucleotide sequence represented by SEQ ID NOS: 170893, 170894, 170895, 170896, 170897, 170898, 170899, 170900, 170901, 170902, 170903 were selected from the ORFs having the nucleotide sequence represented by SEQ ID NO: 166840.

The probes having the nucleotide sequence represented by SEQ ID NOS: 170904, 170905, 170906, 170907, 170908, 170909, 170910, 170911, 170912, 170913, 170914 were selected from the ORFs having the nucleotide sequence represented by SEQ ID NO: 166841.

The probes having the nucleotide sequence represented by SEQ ID NOS: 170915, 170916, 170917, 170918, 170919, 170920, 170921, 170922, 170923, 170924, 170925 were selected from the ORFs having the nucleotide sequence represented by SEQ ID NO: 166842.

The probes having the nucleotide sequence represented by SEQ ID NOS: 170926, 170927, 170928, 170929, 170930, 170931, 170932, 170933, 170934, 170935, 170936 were selected from the ORFs having the nucleotide sequence represented by SEQ ID NO: 166843.

The probes having the nucleotide sequence represented by SEQ ID NOS: 170937, 170938, 170939, 170940, 170941, 170942, 170943, 170944, 170945, 170946, 170947 were selected from the ORFs having the nucleotide sequence represented by SEQ ID NO: 166844.

The probes having the nucleotide sequence represented by SEQ ID NOS: 170948, 170949, 170950, 170951, 170952, 170953, 170954, 170955, 170956, 170957, 170958 were selected from the ORFs having the nucleotide sequence represented by SEQ ID NO: 166845.

The probes having the nucleotide sequence represented by SEQ ID NOS: 170959, 170960, 170961, 170962, 170963, 170964, 170965, 170966, 170967, 170968, 170969 were selected from the ORFs having the nucleotide sequence represented by SEQ ID NO: 166846.

The probes having the nucleotide sequence represented by SEQ ID NOS: 170970, 170971, 170972, 170973, 170974, 170975, 170976, 170977, 170978, 170979, 170980 were selected from the ORFs having the nucleotide sequence represented by SEQ ID NO: 166847.

The probes having the nucleotide sequence represented by SEQ ID NOS: 170981, 170982, 170983, 170984, 170985, 170986, 170987, 170988, 170989, 170990, 170991 were selected from the ORFs having the nucleotide sequence represented by SEQ ID NO: 166848.

The probes having the nucleotide sequence represented by SEQ ID NOS: 170992, 170993, 170994, 170995, 170996, 170997, 170998, 170999, 171000, 171001, 171002 were selected from the ORFs having the nucleotide sequence represented by SEQ ID NO: 166849.

The probes having the nucleotide sequence represented by SEQ ID NOS: 171003, 171004, 171005, 171006, 171007, 171008, 171009, 171010, 171011, 171012, 171013 were selected from the ORFs having the nucleotide sequence represented by SEQ ID NO: 166850.

The probes having the nucleotide sequence represented by SEQ ID NOS: 171014, 171015, 171016, 171017, 171018, 171019, 171020, 171021, 171022, 171023, 171024 were selected from the ORFs having the nucleotide sequence represented by SEQ ID NO: 166851.

The probes having the nucleotide sequence represented by SEQ ID NOS: 171025, 171026, 171027, 171028, 171029, 171030, 171031, 171032, 171033, 171034, 171035 were selected from the ORFs having the nucleotide sequence represented by SEQ ID NO: 166852.

The probes having the nucleotide sequence represented by SEQ ID NOS: 171036, 171037, 171038, 171039, 171040, 171041, 171042, 171043, 171044, 171045, 171046 were selected from the ORFs having the nucleotide sequence represented by SEQ ID NO: 166853.

The probes having the nucleotide sequence represented by SEQ ID NOS: 171047, 171048, 171049, 171050, 171051, 171052, 171053, 171054, 171055, 171056, 171057 were selected from the ORFs having the nucleotide sequence represented by SEQ ID NO: 166854.

The probes having the nucleotide sequence represented by SEQ ID NOS: 171058, 171059, 171060, 171061, 171062, 171063, 171064, 171065, 171066, 171067, 171068 were selected from the ORFs having the nucleotide sequence represented by SEQ ID NO: 166855.

The probes having the nucleotide sequence represented by SEQ ID NOS: 171069, 171070, 171071, 171072, 171073, 171074, 171075, 171076, 171077, 171078, 171079 were selected from the ORFs having the nucleotide sequence represented by SEQ ID NO: 166856.

The probes having the nucleotide sequence represented by SEQ ID NOS: 171080, 171081, 171082, 171083, 171084, 171085, 171086, 171087, 171088, 171089, 171090 were selected from the ORFs having the nucleotide sequence represented by SEQ ID NO: 166857.

The probes having the nucleotide sequence represented by SEQ ID NOS: 171091, 171092, 171093, 171094, 171095, 171096, 171097, 171098, 171099, 171100, 171101 were selected from the ORFs having the nucleotide sequence represented by SEQ ID NO: 166858.

The probes having the nucleotide sequence represented by SEQ ID NOS: 171102, 171103, 171104, 171105, 171106, 171107, 171108, 171109, 171110, 171111, 171112 were selected from the ORFs having the nucleotide sequence represented by SEQ ID NO: 166859.

The probes having the nucleotide sequence represented by SEQ ID NOS: 171113, 171114, 171115, 171116, 171117, 171118, 171119, 171120, 171121, 171122, 171123 were selected from the ORFs having the nucleotide sequence represented by SEQ ID NO: 166860.

The probes having the nucleotide sequence represented by SEQ ID NOS: 171124, 171125, 171126, 171127, 171128, 171129, 171130, 171131, 171132, 171133, 171134 were selected from the ORFs having the nucleotide sequence represented by SEQ ID NO: 166861.

The probes having the nucleotide sequence represented by SEQ ID NOS: 171135, 171136, 171137, 171138, 171139, 171140, 171141, 171142, 171143, 171144, 171145 were selected from the ORFs having the nucleotide sequence represented by SEQ ID NO: 166862.

The probes having the nucleotide sequence represented by SEQ ID NOS: 171146, 171147, 171148, 171149, 171150, 171151, 171152, 171153, 171154, 171155, 171156 were selected from the ORFs having the nucleotide sequence represented by SEQ ID NO: 166863

The probes having the nucleotide sequence represented by SEQ ID NOS: 171157, 171158, 171159, 171160, 171161, 171162, 171163, 171164, 171165, 171166, 171167 were selected from the ORFs having the nucleotide sequence represented by SEQ ID NO: 166864.

The probes having the nucleotide sequence represented by SEQ ID NOS: 171168, 171169, 171170, 171171, 171172, 171173, 171174, 171175, 171176, 171177, 171178 were selected from the ORFs having the nucleotide sequence represented by SEQ ID NO: 166865.

The probes having the nucleotide sequence represented by SEQ ID NOS: 171179, 171180, 171181, 171182, 171183, 171184, 171185, 171186, 171187, 171188, 171189 were selected from the ORFs having the nucleotide sequence represented by SEQ ID NO: 166866.

The probes having the nucleotide sequence represented by SEQ ID NOS: 171190, 171191, 171192, 171193, 171194, 171195, 171196, 171197, 171198, 171199, 171200 were selected from the ORFs having the nucleotide sequence represented by SEQ ID NO: 166867.

The probes having the nucleotide sequence represented by SEQ ID NOS: 171201, 171202, 171203, 171204, 171205, 171206, 171207, 171208, 171209, 171210, 171211 were selected from the ORFs having the nucleotide sequence represented by SEQ ID NO: 166868.

The probes having the nucleotide sequence represented by SEQ ID NOS: 171212, 171213, 171214, 171215, 171216, 171217, 171218, 171219, 171220, 171221, 171222 were selected from the ORFs having the nucleotide sequence represented by SEQ ID NO: 166869.

The probes having the nucleotide sequence represented by SEQ ID NOS: 171223, 171224, 171225, 171226, 171227, 171228, 171229, 171230, 171231, 171232, 171233 were selected from the ORFs having the nucleotide sequence represented by SEQ ID NO: 166870.

The probes having the nucleotide sequence represented by SEQ ID NOS: 171234, 171235, 171236, 171237, 171238, 171239, 171240, 171241, 171242, 171243, 171244 were selected from the ORFs having the nucleotide sequence represented by SEQ ID NO: 166871.

The probes having the nucleotide sequence represented by SEQ ID NOS: 171245, 171246, 171247, 171248, 171249, 171250, 171251, 171252, 171253, 171254, 171255 were selected from the ORFs having the nucleotide sequence represented by SEQ ID NO: 166872.

The probes having the nucleotide sequence represented by SEQ ID NOS: 171256, 171257, 171258, 171259, 171260, 171261, 171262, 171263, 171264, 171265, 171266 were selected from the ORFs having the nucleotide sequence represented by SEQ ID NO: 166873.

The probes having the nucleotide sequence represented by SEQ ID NOS: 171267, 171268, 171269, 171270, 171271, 171272, 171273, 171274, 171275, 171276, 171277 were selected from the ORFs having the nucleotide sequence represented by SEQ ID NO: 166874.

The probes having the nucleotide sequence represented by SEQ ID NOS: 171278, 171279, 171280, 171281, 171282, 171283, 171284, 171285, 171286, 171287, 171288 were selected from the ORFs having the nucleotide sequence represented by SEQ ID NO: 166875.

The probes having the nucleotide sequence represented by SEQ ID NOS: 171289, 171290, 171291, 171292, 171293, 171294, 171295, 171296, 171297, 171298, 171299 were selected from the ORFs having the nucleotide sequence represented by SEQ ID NO: 166876.

The probes having the nucleotide sequence represented by SEQ ID NOS: 171300, 171301, 171302, 171303, 171304, 171305, 171306, 171307, 171308, 171309, 171310 were selected from the ORFs having the nucleotide sequence represented by SEQ ID NO: 166877.

The probes having the nucleotide sequence represented by SEQ ID NOS: 171311, 171312, 171313, 171314, 171315, 171316, 171317, 171318, 171319, 171320, 171321 were selected from the ORFs having the nucleotide sequence represented by SEQ ID NO: 166878.

The probes having the nucleotide sequence represented by SEQ ID NOS: 171322, 171323, 171324, 171325, 171326, 171327, 171328, 171329, 171330, 171331, 171332 were selected from the ORFs having the nucleotide sequence represented by SEQ ID NO: 166879.

The probes having the nucleotide sequence represented by SEQ ID NOS: 171333, 171334, 171335, 171336, 171337, 171338, 171339, 171340, 171341, 171342, 171343 were selected from the ORFs having the nucleotide sequence represented by SEQ ID NO: 166880.

The probes having the nucleotide sequence represented by SEQ ID NOS: 171344, 171345, 171346, 171347, 171348, 171349, 171350, 171351, 171352, 171353 were selected from the ORFs having the nucleotide sequence represented by SEQ ID NO: 166881.

The probes having the nucleotide sequence represented by SEQ ID NOS: 171354, 171355, 171356, 171357, 171358, 171359, 171360, 171361, 171362, 171363, 171364 were selected from the ORFs having the nucleotide sequence represented by SEQ ID NO: 166882.

The probes having the nucleotide sequence represented by SEQ ID NOS: 171365, 171366, 171367, 171368, 171369, 171370, 171371, 171372, 171373, 171374, 171375 were selected from the ORFs having the nucleotide sequence represented by SEQ ID NO: 166883.

The probes having the nucleotide sequence represented by SEQ ID NOS: 171376, 171377, 171378, 171379, 171380, 171381, 171382, 171383, 171384, 171385, 171386 were selected from the ORFs having the nucleotide sequence represented by SEQ ID NO: 166884.

The probes having the nucleotide sequence represented by SEQ ID NOS: 171387, 171388, 171389, 171390, 171391, 171392, 171393, 171394, 171395, 171396, 171397 were selected from the ORFs having the nucleotide sequence represented by SEQ ID NO: 166885.

The probes having the nucleotide sequence represented by SEQ ID NOS: 171398, 171399, 171400, 171401, 171402, 171403, 171404, 171405, 171406, 171407, 171408 were selected from the ORFs having the nucleotide sequence represented by SEQ ID NO: 166886.

The probes having the nucleotide sequence represented by SEQ ID NOS: 171409, 171410, 171411, 171412, 171413, 171414, 171415, 171416, 171417, 171418, 171419 were selected from the ORFs having the nucleotide sequence represented by SEQ ID NO: 166887.

The probes having the nucleotide sequence represented by SEQ ID NOS: 171420, 171421, 171422, 171423, 171424, 171425, 171426, 171427, 171428, 171429, 171430 were selected from the ORFs having the nucleotide sequence represented by SEQ ID NO: 166888.

The probes having the nucleotide sequence represented by SEQ ID NOS: 171431, 171432, 171433, 171434, 171435, 171436, 171437, 171438, 171439, 171440, 171441 were selected from the ORFs having the nucleotide sequence represented by SEQ ID NO: 166889.

The probes having the nucleotide sequence represented by SEQ ID NOS: 171442, 171443, 171444, 171445, 171446, 171447, 171448, 171449, 171450, 171451, 171452 were selected from the ORFs having the nucleotide sequence represented by SEQ ID NO: 166890.

The probes having the nucleotide sequence represented by SEQ ID NOS: 171453, 171454, 171455, 171456, 171457, 171458, 171459, 171460, 171461, 171462, 171463 were selected from the ORFs having the nucleotide sequence represented by SEQ ID NO: 166891.

The probes having the nucleotide sequence represented by SEQ ID NOS: 171464, 171465, 171466, 171467, 171468, 171469, 171470, 171471, 171472, 171473, 171474 were selected from the ORFs having the nucleotide sequence represented by SEQ ID NO: 166892.

The probes having the nucleotide sequence represented by SEQ ID NOS: 171475, 171476, 171477, 171478, 171479, 171480, 171481, 171482, 171483, 171484, 171485 were selected from the ORFs having the nucleotide sequence represented by SEQ ID NO: 166893.

The probes having the nucleotide sequence represented by SEQ ID NOS: 171486, 171487, 171488, 171489, 171490, 171491, 171492, 171493, 171494, 171495, 171496 were selected from the ORFs having the nucleotide sequence represented by SEQ ID NO: 166894.

The probes having the nucleotide sequence represented by SEQ ID NOS: 171497, 171498, 171499, 171500, 171501, 171502, 171503, 171504, 171505, 171506, 171507 were selected from the ORFs having the nucleotide sequence represented by SEQ ID NO: 166895.

The probes having the nucleotide sequence represented by SEQ ID NOS: 171508, 171509, 171510, 171511, 171512, 171513, 171514, 171515, 171516, 171517, 171518 were selected from the ORFs having the nucleotide sequence represented by SEQ ID NO: 166896.

The probes having the nucleotide sequence represented by SEQ ID NOS: 171519, 171520, 171521, 171522, 171523, 171524, 171525, 171526, 171527, 171528, 171529 were selected from the ORFs having the nucleotide sequence represented by SEQ ID NO: 166897.

The probes having the nucleotide sequence represented by SEQ ID NOS: 171530, 171531, 171532, 171533, 171534, 171535, 171536, 171537, 171538, 171539, 171540 were selected from the ORFs having the nucleotide sequence represented by SEQ ID NO: 166898.

The probes having the nucleotide sequence represented by SEQ ID NOS: 171541, 171542, 171543, 171544, 171545, 171546, 171547, 171548, 171549, 171550, 171551 were selected from the ORFs having the nucleotide sequence represented by SEQ ID NO: 166899.

The probes having the nucleotide sequence represented by SEQ ID NOS: 171552, 171553, 171554, 171555, 171556, 171557, 171558, 171559, 171560, 171561, 171562 were selected from the ORFs having the nucleotide sequence represented by SEQ ID NO: 166900.

The probes having the nucleotide sequence represented by SEQ ID NOS: 171563, 171564, 171565, 171566, 171567, 171568, 171569, 171570, 171571, 171572, 171573 were selected from the ORFs having the nucleotide sequence represented by SEQ ID NO: 166901.

The probes having the nucleotide sequence represented by SEQ ID NOS: 171574, 171575, 171576, 171577, 171578, 171579, 171580, 171581, 171582, 171583, 171584 were selected from the ORFs having the nucleotide sequence represented by SEQ ID NO: 166902.

The probes having the nucleotide sequence represented by SEQ ID NOS: 171585, 171586, 171587, 171588, 171589, 171590, 171591, 171592, 171593, 171594, 171595 were selected from the ORFs having the nucleotide sequence represented by SEQ ID NO: 166903.

The probes having the nucleotide sequence represented by SEQ ID NOS: 171596, 171597, 171598, 171599, 171600, 171601, 171602, 171603, 171604, 171605, 171606 were selected from the ORFs having the nucleotide sequence represented by SEQ ID NO: 166904.

The probes having the nucleotide sequence represented by SEQ ID NOS: 171607, 171608, 171609, 171610, 171611, 171612, 171613, 171614, 171615, 171616, 171617 were selected from the ORFs having the nucleotide sequence represented by SEQ ID NO: 166905.

The probes having the nucleotide sequence represented by SEQ ID NOS: 171618, 171619, 171620, 171621, 171622, 171623, 171624, 171625, 171626, 171627, 171628 were selected from the ORFs having the nucleotide sequence represented by SEQ ID NO: 166906.

The probes having the nucleotide sequence represented by SEQ ID NOS: 171629, 171630, 171631, 171632, 171633, 171634, 171635, 171636, 171637, 171638, 171639 were selected from the ORFs having the nucleotide sequence represented by SEQ ID NO: 166907.

The probes having the nucleotide sequence represented by SEQ ID NOS: 171640, 171641, 171642, 171643, 171644, 171645, 171646, 171647, 171648, 171649, 171650 were selected from the ORFs having the nucleotide sequence represented by SEQ ID NO: 166908.

The probes having the nucleotide sequence represented by SEQ ID NOS: 171651, 171652, 171653, 171654, 171655, 171656, 171657, 171658, 171659, 171660, 171661 were selected from the ORFs having the nucleotide sequence represented by SEQ ID NO: 166909.

The probes having the nucleotide sequence represented by SEQ ID NOS: 171662, 171663, 171664, 171665, 171666, 171667, 171668, 171669, 171670, 171671, 171672 were selected from the ORFs having the nucleotide sequence represented by SEQ ID NO: 166910.

The probes having the nucleotide sequence represented by SEQ ID NOS: 171673, 171674, 171675, 171676, 171677, 171678, 171679, 171680, 171681, 171682, 171683 were selected from the ORFs having the nucleotide sequence represented by SEQ ID NO: 166911.

The probes having the nucleotide sequence represented by SEQ ID NOS: 171684, 171685, 171686, 171687, 171688, 171689, 171690, 171691, 171692, 171693, 171694 were selected from the ORFs having the nucleotide sequence represented by SEQ ID NO: 166912.

The probes having the nucleotide sequence represented by SEQ ID NOS: 171695, 171696, 171697, 171698, 171699, 171700, 171701, 171702, 171703, 171704, 171705 were selected from the ORFs having the nucleotide sequence represented by SEQ. ID NO: 166913.

The probes having the nucleotide sequence represented by SEQ ID NOS: 171706, 171707, 171708, 171709, 171710, 171711, 171712, 171713, 171714, 171715, 171716 were selected from the ORFs having the nucleotide sequence represented by SEQ ID NO: 166914.

The probes having the nucleotide sequence represented by SEQ ID NOS: 171717, 171718, 171719, 171720, 171721, 171722, 171723, 171724, 171725, 171726, 171727 were selected from the ORFs having the nucleotide sequence represented by SEQ ID NO: 166915.

The probes having the nucleotide sequence represented by SEQ ID NOS: 171728, 171729, 171730, 171731, 171732, 171733, 171734, 171735, 171736, 171737, 171738 were selected from the ORFs having the nucleotide sequence represented by SEQ ID NO: 166916.

The probes having the nucleotide sequence represented by SEQ ID NOS: 171739, 171740, 171741, 171742, 171743, 171744, 171745, 171746, 171747, 171748, 171749 were selected from the ORFs having the nucleotide sequence represented by SEQ ID NO: 166917.

The probes having the nucleotide sequence represented by SEQ ID NOS: 171750, 171751, 171752, 171753, 171754, 171755, 171756, 171757, 171758, 171759, 171760 were selected from the ORFs having the nucleotide sequence represented by SEQ ID NO: 166918.

The probes having the nucleotide sequence represented by SEQ ID NOS: 171761, 171762, 171763, 171764, 171765, 171766, 171767, 171768, 171769, 171770, 171771 were selected from the ORFs having the nucleotide sequence represented by SEQ ID NO: 166919.

The probes having the nucleotide sequence represented by SEQ ID NOS: 171772, 171773, 171774, 171775, 171776, 171777, 171778, 171779, 171780, 171781, 171782 were selected from the ORFs having the nucleotide sequence represented by SEQ ID NO: 166920.

The probes having the nucleotide sequence represented by SEQ ID NOS: 171783, 171784, 171785, 171786, 171787, 171788, 171789, 171790, 171791, 171792, 171793 were selected from the ORFs having the nucleotide sequence represented by SEQ ID NO: 166921.

The probes having the nucleotide sequence represented by SEQ ID NOS: 171794, 171795, 171796, 171797, 171798, 171799, 171800, 171801, 171802, 171803, 171804 were selected from the ORFs having the nucleotide sequence represented by SEQ ID NO: 166922.

The probes having the nucleotide sequence represented by SEQ ID NOS: 171805, 171806, 171807, 171808, 171809, 171810, 171811, 171812, 171813, 171814, 171815 were selected from the ORFs having the nucleotide sequence represented by SEQ ID NO: 166923.

The probes having the nucleotide sequence represented by SEQ ID NOS: 171816, 171817, 171818, 171819, 171820, 171821, 171822, 171823, 171824, 171825, 171826 were selected from the ORFs having the nucleotide sequence represented by SEQ ID NO: 166924.

The probes having the nucleotide sequence represented by SEQ ID NOS: 171827, 171828, 171829, 171830, 171831, 171832, 171833, 171834, 171835, 171836, 171837 were selected from the ORFs having the nucleotide sequence represented by SEQ ID NO: 166925.

The probes having the nucleotide sequence represented by SEQ ID NOS: 171838, 171839, 171840, 171841, 171842, 171843, 171844, 171845, 171846, 171847, 171848 were selected from the ORFs having the nucleotide sequence represented by SEQ ID NO: 166926.

The probes having the nucleotide sequence represented by SEQ ID NOS: 171849, 171850, 171851, 171852, 171853, 171854, 171855, 171856, 171857, 171858, 171859 were selected from the ORFs having the nucleotide sequence represented by SEQ ID NO: 166927.

The probes having the nucleotide sequence represented by SEQ ID NOS: 171860, 171861, 171862, 171863, 171864, 171865, 171866, 171867, 171868, 171869, 171870 were selected from the ORFs having the nucleotide sequence represented by SEQ ID NO: 166928. The probes having the nucleotide sequence represented by SEQ ID NOS: 171871, 171872, 171873, 171874, 171875, 171876, 171877, 171878, 171879, 171880, 171881 were selected from the ORFs having the nucleotide sequence represented by SEQ ID NO: 166929.

The probes having the nucleotide sequence represented by SEQ ID NOS: 171882, 171883, 171884, 171885, 171886, 171887, 171888, 171889, 171890, 171891, 171892 were selected from the ORFs having the nucleotide sequence represented by SEQ ID NO: 166930.

The probes having the nucleotide sequence represented by SEQ ID NOS: 171893, 171894, 171895, 171896, 171897, 171898, 171899, 171900, 171901, 171902, 171903 were selected from the ORFs having the nucleotide sequence represented by SEQ ID NO: 166931.

The probes having the nucleotide sequence represented by SEQ ID NOS: 171904, 171905, 171906, 171907, 171908, 171909, 171910, 171911, 171912, 171913, 171914 were selected from the ORFs having the nucleotide sequence represented by SEQ ID NO: 166932.

The probes having the nucleotide sequence represented by SEQ ID NOS: 171915, 171916, 171917, 171918, 171919, 171920, 171921, 171922, 171923, 171924, 171925 were selected from the ORFs having the nucleotide sequence represented by SEQ ID NO: 166933.

The probes having the nucleotide sequence represented by SEQ ID NOS: 171926, 171927, 171928, 171929, 171930, 171931, 171932, 171933, 171934, 171935, 171936 were selected from the ORFs having the nucleotide sequence represented by SEQ ID NO: 166934.

The probes having the nucleotide sequence represented by SEQ ID NOS: 171937, 171938, 171939, 171940, 171941, 171942, 171943, 171944, 171945, 171946, 171947 were selected from the ORFs having the nucleotide sequence represented by SEQ ID NO: 166935.

The probes having the nucleotide sequence represented by SEQ ID NOS: 171948, 171949, 171950, 171951, 171952, 171953, 171954, 171955, 171956, 171957, 171958 were selected from the ORFs having the nucleotide sequence represented by SEQ ID NO: 166936.

The probes having the nucleotide sequence represented by SEQ ID NOS: 171959, 171960, 171961, 171962, 171963, 171964, 171965, 171966, 171967, 171968, 171969 were selected from the ORFs having the nucleotide sequence represented by SEQ ID NO: 166937.

The probes having the nucleotide sequence represented by SEQ ID NOS: 171970, 171971, 171972, 171973, 171974, 171975, 171976, 171977, 171978, 171979, 171980 were selected from the ORFs having the nucleotide sequence represented by SEQ ID NO: 166938.

The probes having the nucleotide sequence represented by SEQ ID NOS: 171981, 171982, 171983, 171984, 171985, 171986, 171987, 171988, 171989, 171990, 171991 were selected from the ORFs having the nucleotide sequence represented by SEQ ID NO: 166939.

The probes having the nucleotide sequence represented by SEQ ID NOS: 171992, 171993, 171994, 171995, 171996, 171997, 171998, 171999, 172000, 172001, 172002 were selected from the ORFs having the nucleotide sequence represented by SEQ ID NO: 166940.

The probes having the nucleotide sequence represented by SEQ ID NOS: 172003, 172004, 172005, 172006, 172007, 172008, 172009, 172010, 172011, 172012, 172013 were selected from the ORFs having the nucleotide sequence represented by SEQ ID NO: 166941.

The probes having the nucleotide sequence represented by SEQ ID NOS: 172014, 172015, 172016, 172017, 172018, 172019, 172020, 172021, 172022, 172023, 172024 were selected from the ORFs having the nucleotide sequence represented by SEQ ID NO: 166942.

The probes having the nucleotide sequence represented by SEQ ID NOS: 172025, 172026, 172027, 172028, 172029, 172030, 172031, 172032, 172033, 172034, 172035 were selected from the ORFs having the nucleotide sequence represented by SEQ ID NO: 166943.

The probes having the nucleotide sequence represented by SEQ ID NOS: 172036, 172037, 172038, 172039, 172040, 172041, 172042, 172043, 172044, 172045, 172046 were selected from the ORFs having the nucleotide sequence represented by SEQ ID NO: 166944.

The probes having the nucleotide sequence represented by SEQ ID NOS: 172047, 172048, 172049, 172050, 172051, 172052, 172053, 172054, 172055, 172056, 172057 were selected from the ORFs having the nucleotide sequence represented by SEQ ID NO: 166945.

The probes having the nucleotide sequence represented by SEQ ID NOS: 172058, 172059, 172060, 172061, 172062, 172063, 172064, 172065, 172066, 172067, 172068 were selected from the ORFs having the nucleotide sequence represented by SEQ ID NO: 166946.

The probes having the nucleotide sequence represented by SEQ ID NOS: 172069, 172070, 172071, 172072, 172073, 172074, 172075, 172076, 172077, 172078, 172079 were selected from the ORFs having the nucleotide sequence represented by SEQ ID NO: 166947.

The probes having the nucleotide sequence represented by SEQ ID NOS: 172080, 172081, 172082, 172083, 172084, 172085, 172086, 172087, 172088, 172089, 172090 were selected from the ORFs having the nucleotide sequence represented by SEQ ID NO: 166948.

The probes having the nucleotide sequence represented by SEQ ID NOS: 172091, 172092, 172093, 172094, 172095, 172096, 172097, 172098, 172099, 172100, 172101 were selected from the ORFs having the nucleotide sequence represented by SEQ ID NO: 166949.

The probes having the nucleotide sequence represented by SEQ ID NOS: 172102, 172103, 172104, 172105, 172106, 172107, 172108, 172109, 172110, 172111, 172112 were selected from the ORFs having the nucleotide sequence represented by SEQ ID NO: 166950.

The probes having the nucleotide sequence represented by SEQ ID NOS: 172113, 172114, 172115, 172116, 172117, 172118, 172119, 172120, 172121, 172122, 172123 were selected from the ORFs having the nucleotide sequence represented by SEQ ID NO: 166951.

The probes having the nucleotide sequence represented by SEQ ID NOS: 172124, 172125, 172126, 172127, 172128, 172129, 172130, 172131, 172132, 172133, 172134 were selected from the ORFs having the nucleotide sequence represented by SEQ ID NO: 166952.

The probes having the nucleotide sequence represented by SEQ ID NOS: 172135, 172136, 172137, 172138, 172139, 172140, 172141, 172142, 172143, 172144, 172145 were selected from the ORFs having the nucleotide sequence represented by SEQ ID NO: 166953.

The probes having the nucleotide sequence represented by SEQ ID NOS: 172146, 172147, 172148, 172149, 172150, 172151, 172152, 172153, 172154, 172155, 172156 were selected from the ORFs having the nucleotide sequence represented by SEQ ID NO: 166954.

The probes having the nucleotide sequence represented by SEQ ID NOS: 172157, 172158, 172159, 172160, 172161, 172162, 172163, 172164, 172165, 172166, 172167 were selected from the ORFs having the nucleotide sequence represented by SEQ ID NO: 166955.

The probes having the nucleotide sequence represented by SEQ ID NOS: 172168, 172169, 172170, 172171, 172172, 172173, 172174, 172175, 172176, 172177, 172178 were selected from the ORFs having the nucleotide sequence represented by SEQ ID NO: 166956.

The probes having the nucleotide sequence represented by SEQ ID NOS: 172179, 172180, 172181, 172182, 172183, 172184, 172185, 172186, 172187, 172188, 172189 were selected from the ORFs having the nucleotide sequence represented by SEQ ID NO: 166957.

The probes having the nucleotide sequence represented by SEQ ID NOS: 172190, 172191, 172192, 172193, 172194, 172195, 172196, 172197, 172198, 172199, 172200 were selected from the ORFs having the nucleotide sequence represented by SEQ ID NO: 166958.

The probes having the nucleotide sequence represented by SEQ ID NOS: 172201, 172202, 172203, 172204, 172205, 172206, 172207, 172208, 172209, 172210, 172211 were selected from the ORFs having the nucleotide sequence represented by SEQ ID NO: 166959.

The probes having the nucleotide sequence represented by SEQ ID NOS: 172212, 172213, 172214, 172215, 172216, 172217, 172218, 172219, 172220, 172221, 172222 were selected from the ORFs having the nucleotide sequence represented by SEQ ID NO: 166960.

The probes having the nucleotide sequence represented by SEQ ID NOS: 172223, 172224, 172225, 172226, 172227, 172228, 172229, 172230, 172231, 172232, 172233 were selected from the ORFs having the nucleotide sequence represented by SEQ ID NO: 166961.

The probes having the nucleotide sequence represented by SEQ ID NOS: 172234, 172235, 172236, 172237, 172238, 172239, 172240, 172241, 172242, 172243, 172244 were selected from the ORFs having the nucleotide sequence represented by SEQ ID NO: 166962.

The probes having the nucleotide sequence represented by SEQ ID NOS: 172245, 172246, 172247, 172248, 172249, 172250, 172251, 172252, 172253, 172254, 172255 were selected from the ORFs having the nucleotide sequence represented by SEQ ID NO: 166963.

The probes having the nucleotide sequence represented by SEQ ID NOS: 172256, 172257, 172258, 172259, 172260, 172261, 172262, 172263, 172264, 172265, 172266 were selected from the ORFs having the nucleotide sequence represented by SEQ ID NO: 166964.

The probes having the nucleotide sequence represented by SEQ ID NOS: 172267, 172268, 172269, 172270, 172271, 172272, 172273, 172274, 172275, 172276, 172277 were selected from the ORFs having the nucleotide sequence represented by SEQ ID NO: 166965.

The probes having the nucleotide sequence represented by SEQ ID NOS: 172278, 172279, 172280, 172281, 172282, 172283, 172284, 172285, 172286, 172287, 172288 were selected from the ORFs having the nucleotide sequence represented by SEQ ID NO: 166966.

The probes having the nucleotide sequence represented by SEQ ID NOS: 172289, 172290, 172291, 172292, 172293, 172294, 172295, 172296, 172297, 172298, 172299 were selected from the ORFs having the nucleotide sequence represented by SEQ ID NO: 166967.

The probes having the nucleotide sequence represented by SEQ ID NOS: 172300, 172301, 172302, 172303, 172304, 172305, 172306, 172307, 172308, 172309, 172310 were selected from the ORFs having the nucleotide sequence represented by SEQ ID NO: 166968.

The probes having the nucleotide sequence represented by SEQ ID NOS: 172311, 172312, 172313, 172314, 172315, 172316, 172317, 172318, 172319, 172320, 172321 were selected from the ORFs having the nucleotide sequence represented by SEQ ID NO: 166969.

The probes having the nucleotide sequence represented by SEQ ID NOS: 172322, 172323, 172324, 172325, 172326, 172327, 172328, 172329, 172330, 172331, 172332 were selected from the ORFs having the nucleotide sequence represented by SEQ ID NO: 166970.

The probes having the nucleotide sequence represented by SEQ ID NOS: 172333, 172334, 172335, 172336, 172337, 172338, 172339, 172340, 172341, 172342, 172343 were selected from the ORFs having the nucleotide sequence represented by SEQ ID NO: 166971.

The probes having the nucleotide sequence represented by SEQ ID NOS: 172344, 172345, 172346, 172347, 172348, 172349, 172350, 172351, 172352, 172353, 172354 were selected from the ORFs having the nucleotide sequence represented by SEQ ID NO: 166972.

The probes having the nucleotide sequence represented by SEQ ID NOS: 172355, 172356, 172357, 172358, 172359, 172360, 172361, 172362, 172363, 172364, 172365 were selected from the ORFs having the nucleotide sequence represented by SEQ ID NO: 166973.

The probes having the nucleotide sequence represented by SEQ ID NOS: 172366, 172367, 172368, 172369, 172370, 172371, 172372, 172373, 172374, 172375, 172376 were selected from the ORFs having the nucleotide sequence represented by SEQ ID NO: 166974.

The probes having the nucleotide sequence represented by SEQ ID NOS: 172377, 172378, 172379, 172380, 172381, 172382, 172383, 172384, 172385, 172386, 172387 were selected from the ORFs having the nucleotide sequence represented by SEQ ID NO: 166975.

The probes having the nucleotide sequence represented by SEQ ID NOS: 172388, 172389, 172390, 172391, 172392, 172393, 172394, 172395, 172396, 172397, 172398 were selected from the ORFs having the nucleotide sequence represented by SEQ ID NO: 166976.

The probes having the nucleotide sequence represented by SEQ ID NOS: 172399, 172400, 172401, 172402, 172403, 172404, 172405, 172406, 172407, 172408, 172409 were selected from the ORFs having the nucleotide sequence represented by SEQ ID NO: 166977.

The probes having the nucleotide sequence represented by SEQ ID NOS: 172410, 172411, 172412, 172413, 172414, 172415, 172416, 172417, 172418, 172419, 172420 were selected from the ORFs having the nucleotide sequence represented by SEQ ID NO: 166978.

The probes having the nucleotide sequence represented by SEQ ID NOS: 172421, 172422, 172423, 172424, 172425, 172426, 172427, 172428, 172429, 172430, 172431 were selected from the ORFs having the nucleotide sequence represented by SEQ ID NO: 166979.

The probes having the nucleotide sequence represented by SEQ ID NOS: 172432, 172433, 172434, 172435, 172436, 172437, 172438, 172439, 172440, 172441, 172442 were selected from the ORFs having the nucleotide sequence represented by SEQ ID NO: 166980.

The probes having the nucleotide sequence represented by SEQ ID NOS: 172443, 172444, 172445, 172446, 172447, 172448, 172449, 172450, 172451, 172452, 172453 were selected from the ORFs having the nucleotide sequence represented by SEQ ID NO: 166981.

The probes having the nucleotide sequence represented by SEQ ID NOS: 172454, 172455, 172456, 172457, 172458, 172459, 172460, 172461, 172462, 172463, 172464 were selected from the ORFs having the nucleotide sequence represented by SEQ ID NO: 166982.

The probes having the nucleotide sequence represented by SEQ ID NOS: 172465, 172466, 172467, 172468, 172469, 172470, 172471, 172472, 172473, 172474, 172475 were selected from the ORFs having the nucleotide sequence represented by SEQ ID NO: 166983.

The probes having the nucleotide sequence represented by SEQ ID NOS: 172476, 172477, 172478, 172479, 172480, 172481, 172482, 172483, 172484, 172485, 172486 were selected from the ORFs having the nucleotide sequence represented by SEQ ID NO: 166984.

The probes having the nucleotide sequence represented by SEQ ID NOS: 172487, 172488, 172489, 172490, 1724.91, 172492, 172493, 172494, 172495, 172496, 172497 were selected from the ORFs having the nucleotide sequence represented by SEQ ID NO: 166985.

The probes having the nucleotide sequence represented by SEQ ID NOS: 172498, 172499, 172500, 172501, 172502, 172503, 172504, 172505, 172506, 172507, 172508 were selected from the ORFs having the nucleotide sequence represented by SEQ ID NO: 166986.

The probes having the nucleotide sequence represented by SEQ ID NOS: 172509, 172510, 172511, 172512, 172513, 172514, 172515, 172516, 172517, 172518, 172519 were selected from the ORFs having the nucleotide sequence represented by SEQ ID NO: 166987.

The probes having the nucleotide sequence represented by SEQ ID NOS: 172520, 172521, 172522, 172523, 172524, 172525, 172526, 172527, 172528, 172529, 172530 were selected from the ORFs having the nucleotide sequence represented by SEQ ID NO: 166988.

The probes having the nucleotide sequence represented by SEQ ID NOS: 172531, 172532, 172533, 172534, 172535, 172536, 172537, 172538, 172539, 172540, 172541 were selected from the ORFs having the nucleotide sequence represented by SEQ ID NO: 166989.

The probes having the nucleotide sequence represented by SEQ ID NOS: 172542, 172543, 172544, 172545, 172546, 172547, 172548, 172549, 172550, 172551, 172552 were selected from the ORFs having the nucleotide sequence represented by SEQ ID NO: 166990.

The probes having the nucleotide sequence represented by SEQ ID NOS: 172553, 172554, 172555, 172556, 172557, 172558, 172559, 172560, 172561, 172562, 172563 were selected from the ORFs having the nucleotide sequence represented by SEQ ID NO: 166991.

The probes having the nucleotide sequence represented by SEQ ID NOS: 172564, 172565, 172566, 172567, 172568, 172569, 172570, 172571, 172572, 172573, 172574 were selected from the ORFs having the nucleotide sequence represented by SEQ ID NO: 166992.

The probes having the nucleotide sequence represented by SEQ ID NOS: 172575, 172576, 172577, 172578, 172579, 172580, 172581, 172582, 172583, 172584, 172585 were selected from the ORFs having the nucleotide sequence represented by SEQ ID NO: 166993.

The probes having the nucleotide sequence represented by SEQ ID NOS: 172586, 172587, 172588, 172589, 172590, 172591, 172592, 172593, 172594, 172595, 172596 were selected from the ORFs having the nucleotide sequence represented by SEQ ID NO: 166994.

The probes having the nucleotide sequence represented by SEQ ID NOS: 172597, 172598, 172599, 172600, 172601, 172602, 172603, 172604, 172605, 172606, 172607 were selected from the ORFs having the nucleotide sequence represented by SEQ ID NO: 166995.

The probes having the nucleotide sequence represented by SEQ ID NOS: 172608, 172609, 172610, 172611, 172612, 172613, 172614, 172615, 172616, 172617, 172618 were selected from the ORFs having the nucleotide sequence represented by SEQ ID NO: 166996.

The probes having the nucleotide sequence represented by SEQ ID NOS: 172619, 172620, 172621, 172622, 172623, 172624, 172625, 172626, 172627, 172628, 172629 were selected from the ORFs having the nucleotide sequence represented by SEQ ID NO: 166997.

The probes having the nucleotide sequence represented by SEQ ID NOS: 172630, 172631, 172632, 172633, 172634, 172635, 172636, 172637, 172638, 172639, 172640 were selected from the ORFs having the nucleotide sequence represented by SEQ ID NO: 166998.

The probes having the nucleotide sequence represented by SEQ ID NOS: 172641, 172642, 172643, 172644, 172645, 172646, 172647, 172648, 172649, 172650, 172651 were selected from the ORFs having the nucleotide sequence represented by SEQ ID NO: 166999.

The probes having the nucleotide sequence represented by SEQ ID NOS: 172652, 172653, 172654, 172655, 172656, 172657, 172658, 172659, 172660, 172661, 172662 were selected from the ORFs having the nucleotide sequence represented by SEQ ID NO: 167000.

The probes having the nucleotide sequence represented by SEQ ID NOS: 172663, 172664, 172665, 172666, 172667, 172668, 172669, 172670, 172671, 172672, 172673 were selected from the ORFs having the nucleotide sequence represented by SEQ ID NO: 167001.

The probes having the nucleotide sequence represented by SEQ ID NOS: 172674, 172675, 172676, 172677, 172678, 172679, 172680, 172681, 172682, 172683, 172684 were selected from the ORFs having the nucleotide sequence represented by SEQ ID NO: 167002.

The probes having the nucleotide sequence represented by SEQ ID NOS: 172685, 172686, 172687, 172688, 172689, 172690, 172691, 172692, 172693, 172694, 172695 were selected from the ORFs having the nucleotide sequence represented by SEQ ID NO: 167003.

The probes having the nucleotide sequence represented by SEQ ID NOS: 172696, 172697, 172698, 172699, 172700, 172701, 172702, 172703, 172704, 172705, 172706 were selected from the ORFs having the nucleotide sequence represented by SEQ ID NO: 167004.

The probes having the nucleotide sequence represented by SEQ ID NOS: 172707, 172708, 172709, 172710, 172711, 172712, 172713, 172714, 172715, 172716, 172717 were selected from the ORFs having the nucleotide sequence represented by SEQ ID NO: 167005.

The probes having the nucleotide sequence represented by SEQ ID NOS: 172718, 172719, 172720, 172721, 172722, 172723, 172724, 172725, 172726, 172727, 172728 were selected from the ORFs having the nucleotide sequence represented by SEQ ID NO: 167006.

The probes having the nucleotide sequence represented by SEQ ID NOS: 172729, 172730, 172731, 172732, 172733, 172734, 172735, 172736, 172737, 172738, 172739 were selected from the ORFs having the nucleotide sequence represented by SEQ ID NO: 167007.

The probes having the nucleotide sequence represented by SEQ ID NOS: 172740, 172741, 172742, 172743, 172744, 172745, 172746, 172747, 172748, 172749, 172750 were selected from the ORFs having the nucleotide sequence represented by SEQ ID NO: 167008.

The probes having the nucleotide sequence represented by SEQ ID NOS: 172751, 172752, 172753, 172754, 172755, 172756, 172757, 172758, 172759, 172760, 172761 were selected from the ORFs having the nucleotide sequence represented by SEQ ID NO: 167009.

The probes having the nucleotide sequence represented by SEQ ID NOS: 172762, 172763, 172764, 172765, 172766, 172767, 172768, 172769, 172770, 172771, 172772 were selected from the ORFs having the nucleotide sequence represented by SEQ ID NO: 167010.

The probes having the nucleotide sequence represented by SEQ ID NOS: 172773, 172774, 172775, 172776, 172777, 172778, 172779, 172780, 172781, 172782, 172783 were selected from the ORFs having the nucleotide sequence represented by SEQ ID NO: 167011.

The probes having the nucleotide sequence represented by SEQ ID NOS: 172784, 172785, 172786, 172787, 172788, 172789, 172790, 172791, 172792, 172793, 172794 were selected from the ORFs having the nucleotide sequence represented by SEQ ID NO: 167012.

The probes having the nucleotide sequence represented by SEQ ID NOS: 172795, 172796, 172797, 172798, 172799, 172800, 172801, 172802,. 172803, 172804, 172805 were selected from the ORFs having the nucleotide sequence represented by SEQ ID NO: 167013.

The probes having the nucleotide sequence represented by SEQ ID NOS: 172806, 172807, 172808, 172809, 172810, 172811, 172812, 172813, 172814, 172815, 172816 were selected from the ORFs having the nucleotide sequence represented by SEQ ID NO: 167014.

The probes having the nucleotide sequence represented by SEQ ID NOS: 172817, 172818, 172819, 172820, 172821, 172822, 172823, 172824, 172825, 172826, 172827 were selected from the ORFs having the nucleotide sequence represented by SEQ ID NO: 167015.

The probes having the nucleotide sequence represented by SEQ ID NOS: 172828, 172829, 172830, 172831, 172832, 172833, 172834, 172835, 172836, 172837, 172838 were selected from the ORFs having the nucleotide sequence represented by SEQ ID NO: 167016.

The probes having the nucleotide sequence represented by SEQ ID NOS: 172839, 172840, 172841, 172842, 172843, 172844, 172845, 172846, 172847, 172848, 172849 were selected from the ORFs having the nucleotide sequence represented by SEQ ID NO: 167017.

The probes having the nucleotide sequence represented by SEQ ID NOS: 172850, 172851, 172852, 172853, 172854, 172855, 172856, 172857, 172858, 172859, 172860 were selected from the ORFs having the nucleotide sequence represented by SEQ ID NO: 167018.

The probes having the nucleotide sequence represented by SEQ ID NOS: 172861, 172862, 172863, 172864, 172865, 172866, 172867, 172868, 172869, 172870, 172871 were selected from the ORFs having the nucleotide sequence represented by SEQ ID NO: 167019.

The probes having the nucleotide sequence represented by SEQ ID NOS: 172872, 172873, 172874, 172875, 172876, 172877, 172878, 172879, 172880, 172881, 172882 were selected from the ORFs having the nucleotide sequence represented by SEQ ID NO: 167020.

The probes having the nucleotide sequence represented by SEQ ID NOS: 172883, 172884, 172885, 172886, 172887, 172888, 172889, 172890, 172891, 172892, 172893 were selected from the ORFs having the nucleotide sequence represented by SEQ ID NO: 167021.

The probes having the nucleotide sequence represented by SEQ ID NOS: 172894, 172895, 172896, 172897, 172898, 172899, 172900, 172901, 172902, 172903, 172904 were selected from the ORFs having the nucleotide sequence represented by SEQ ID NO: 167022.

The probes having the nucleotide sequence represented by SEQ ID NOS: 172905, 172906, 172907, 172908, 172909, 172910, 172911, 172912, 172913, 172914, 172915 were selected from the ORFs having the nucleotide sequence represented by SEQ ID NO: 167023.

The probes having the nucleotide sequence represented by SEQ ID NOS: 172916, 172917, 172918, 172919, 172920, 172921, 172922, 172923, 172924, 172925, 172926 were selected from the ORFs having the nucleotide sequence represented by SEQ ID NO: 167024.

The probes having the nucleotide sequence represented by SEQ ID NOS: 172927, 172928, 172929, 172930, 172931, 172932, 172933, 172934, 172935, 172936, 172937 were selected from the ORFs having the nucleotide sequence represented by SEQ ID NO: 167025.

The probes having the nucleotide sequence represented by SEQ ID NOS: 172938, 172939, 172940, 172941, 172942, 172943, 172944, 172945, 172946, 172947, 172948 were selected from the ORFs having the nucleotide sequence represented by SEQ ID NO: 167026.

The probes having the nucleotide sequence represented by SEQ ID NOS: 172949, 172950, 172951, 172952, 172953, 172954, 172955, 172956, 172957, 172958, 172959 were selected from the ORFs having the nucleotide sequence represented by SEQ ID NO: 167027.

The probes having the nucleotide sequence represented by SEQ ID NOS: 172960, 172961, 172962, 172963, 172964, 172965, 172966, 172967, 172968, 172969, 172970 were selected from the ORFs having the nucleotide sequence represented by SEQ ID NO: 167028.

The probes having the nucleotide sequence represented by SEQ ID NOS: 172971, 172972, 172973, 172974, 172975, 172976, 172977, 172978, 172979, 172980, 172981 were selected from the ORFs having the nucleotide sequence represented by SEQ ID NO: 167029.

The probes having the nucleotide sequence represented by SEQ ID NOS: 172982, 172983, 172984, 172985, 172986, 172987, 172988, 172989, 172990, 172991, 172992 were selected from the ORFs having the nucleotide sequence represented by SEQ ID NO: 167030.

The probes having the nucleotide sequence represented by SEQ ID NOS: 172993, 172994, 172995, 172996, 172997, 172998, 172999, 173000, 173001, 173002, 173003 were selected from the ORFs having the nucleotide sequence represented by SEQ ID NO: 167031.

The probes having the nucleotide sequence represented by SEQ ID NOS: 173004, 173005, 173006, 173007, 173008, 173009, 173010, 173011, 173012, 173013, 173014 were selected from the ORFs having the nucleotide sequence represented by SEQ ID NO: 167032.

The probes having the nucleotide sequence represented by SEQ ID NOS: 173015, 173016, 173017, 173018, 173019, 173020, 173021, 173022, 173023, 173024, 173025 were selected from the ORFs having the nucleotide sequence represented by SEQ ID NO: 167033.

The probes having the nucleotide sequence represented by SEQ ID NOS: 173026, 173027, 173028, 173029, 173030, 173031, 173032, 173033, 173034, 173035, 173036 were selected from the ORFs having the nucleotide sequence represented by SEQ ID NO: 167034.

The probes having the nucleotide sequence represented by SEQ ID NOS: 173037, 173038, 173039, 173040, 173041, 173042, 173043, 173044, 173045, 173046, 173047 were selected from the ORFs having the nucleotide sequence represented by SEQ ID NO: 167035.

The probes having the nucleotide sequence represented by SEQ ID NOS: 173048, 173049, 173050, 173051, 173052, 173053, 173054, 173055, 173056, 173057, 173058 were selected from the ORFs having the nucleotide sequence represented by SEQ ID NO: 167036.

The probes having the nucleotide sequence represented by SEQ ID NOS: 173059, 173060, 173061, 173062, 173063, 173064, 173065, 173066, 173067, 173068, 173069 were selected from the ORFs having the nucleotide sequence represented by SEQ ID NO: 167037.

The probes having the nucleotide sequence represented by SEQ ID NOS: 173070, 173071, 173072, 173073, 173074, 173075, 173076, 173077, 173078, 173079, 173080 were selected from the ORFs having the nucleotide sequence represented by SEQ ID NO: 167038.

The probes having the nucleotide sequence represented by SEQ ID NOS: 173081, 173082, 173083, 173084, 173085, 173086, 173087, 173088, 173089, 173090, 173091 were selected from the ORFs having the nucleotide sequence represented by SEQ ID NO: 167039.

The probes having the nucleotide sequence represented by SEQ ID NOS: 173092, 173093, 173094, 173095, 173096, 173097, 173098, 173099, 173100, 173101, 173102 were selected from the ORFs having the nucleotide sequence represented by SEQ ID NO: 167040.

The probes having the nucleotide sequence represented by SEQ ID NOS: 173103, 173104, 173105, 173106, 173107, 173108, 173109, 173110, 173111, 173112, 173113 were selected from the ORFs having the nucleotide sequence represented by SEQ ID NO: 167041.

The probes having the nucleotide sequence represented by SEQ ID NOS: 173114, 173115, 173116, 173117, 173118, 173119, 173120, 173121, 173122, 173123, 173124 were selected from the ORFs having the nucleotide sequence represented by SEQ ID NO: 167042.

The probes from 1479 ORF sequences described as above (1-E) were as follows:

The probes having the nucleotide sequence represented by SEQ ID NOS: 174604, 174605, 174606, 174607, 174608, 174609, 174610, 174611, 174612, 174613, 174614 were selected from the ORFs having the nucleotide sequence represented by SEQ ID NO: 173125.

The probes having the nucleotide sequence represented by SEQ ID NOS: 174615, 174616, 174617, 174618, 174619, 174620, 174621, 174622, 174623, 174624, 174625 were selected from the ORFs having the nucleotide sequence represented by SEQ ID NO: 173126.

The probes having the nucleotide sequence represented by SEQ ID NOS: 174626, 174627, 174628, 174629, 174630, 174631, 174632, 174633, 174634, 174635, 174636 were selected from the ORFs having the nucleotide sequence represented by SEQ ID NO: 173127.

The probes having the nucleotide sequence represented by SEQ ID NOS: 174637, 174638, 174639, 174640, 174641, 174642, 174643, 174644, 174645, 174646, 174647 were selected from the ORFs having the nucleotide sequence represented by SEQ ID NO: 173128.

The probes having the nucleotide sequence represented by SEQ ID NOS: 174648, 174649, 174650, 174651, 174652, 174653, 174654, 174655, 174656, 174657, 174658 were selected from the ORFs having the nucleotide sequence represented by SEQ ID NO: 173129.

The probes having the nucleotide sequence represented by SEQ ID NOS: 174659, 174660, 174661, 174662, 174663, 174664, 174665, 174666, 174667, 174668 were selected from the ORFs having the nucleotide sequence represented by SEQ ID NO: 173130.

The probes having the nucleotide sequence represented by SEQ ID NOS: 174669, 174670, 174671, 174672, 174673, 174674, 174675, 174676, 174677, 174678, 174679 were selected from the ORFs having the nucleotide sequence represented by SEQ ID NO: 173131.

The probes having the nucleotide sequence represented by SEQ ID NOS: 174680, 174681, 174682, 174683, 174684, 174685, 174686, 174687, 174688, 174689, 174690 were selected from the ORFs having the nucleotide sequence represented by SEQ ID NO: 173132.

The probes having the nucleotide sequence represented by SEQ ID NOS: 174691, 174692, 174693, 174694, 174695, 174696, 174697, 174698, 174699, 174700, 174701 were selected from the ORFs having the nucleotide sequence represented by SEQ ID NO: 173133.

The probes having the nucleotide sequence represented by SEQ ID NOS: 174702, 174703, 174704, 174705, 174706, 174707, 174708, 174709, 174710, 174711, 174712 were selected from the ORFs having the nucleotide sequence represented by SEQ ID NO: 173134.

The probes having the nucleotide sequence represented by SEQ ID NOS: 174713, 174714, 174715, 174716, 174717, 174718, 174719, 174720, 174721, 174722, 174723 were selected from the ORFs having the nucleotide sequence represented by SEQ ID NO: 173135.

The probes having the nucleotide sequence represented by SEQ ID NOS: 174724, 174725, 174726, 174727, 174728, 174729, 174730, 174731, 174732, 174733, 174734 were selected from the ORFs having the nucleotide sequence represented by SEQ ID NO: 173136.

The probes having the nucleotide sequence represented by SEQ ID NOS: 174735, 174736, 174737, 174738, 174739, 174740, 174741, 174742, 174743, 174744 were selected from the ORFs having the nucleotide sequence represented by SEQ ID NO: 173137.

The probes having the nucleotide sequence represented by SEQ ID NOS: 174745, 174746, 174747, 174748, 174749, 174750, 174751, 174752, 174753, 174754, 174755 were selected from the ORFs having the nucleotide sequence represented by SEQ ID NO: 173138.

The probes having the nucleotide sequence represented by SEQ ID NOS: 174756, 174757, 174758, 174759, 174760, 174761, 174762, 174763, 174764, 174765, 174766 were selected from the ORFs having the nucleotide sequence represented by SEQ ID NO: 173139.

The probes having the nucleotide sequence represented by SEQ ID NOS: 174767, 174768, 174769, 174770, 174771, 174772, 174773, 174774, 174775, 174776, 174777 were selected from the ORFs having the nucleotide sequence represented by SEQ ID NO: 173140.

The probes having the nucleotide sequence represented by SEQ ID NOS: 174778, 174779, 174780, 174781, 174782, 174783, 174784, 174785, 174786, 174787, 174788 were selected from the ORFs having the nucleotide sequence represented by SEQ ID NO: 173141.

The probes having the nucleotide sequence represented by SEQ ID NOS: 174789, 174790, 174791, 174792, 174793, 174794, 174795, 174796, 174797, 174798, 174799 were selected from the ORFs having the nucleotide sequence represented by SEQ ID NO: 173142.

The probes having the nucleotide sequence represented by SEQ ID NOS: 174800, 174801, 174802, 174803, 174804, 174805, 174806, 174807, 174808, 174809, 174810 were selected from the ORFs having the nucleotide sequence represented by SEQ ID NO: 173143.

The probes having the nucleotide sequence represented by SEQ ID NOS: 174811, 174812, 174813, 174814, 174815, 174816, 174817, 174818, 174819, 174820, 174821 were selected from the ORFs having the nucleotide sequence represented by SEQ ID NO: 173144.

The probes having the nucleotide sequence represented by SEQ ID NOS: 174822, 174823, 174824, 174825, 174826, 174827, 174828, 174829, 174830, 174831, 174832 were selected from the ORFs having the nucleotide sequence represented by SEQ ID NO: 173145.

The probes having the nucleotide sequence represented by SEQ ID NOS: 174833, 174834, 174835, 174836, 174837, 174838, 174839, 174840, 174841, 174842, 174843 were selected from the ORFs having the nucleotide sequence represented by SEQ ID NO: 173146.

The probes having the nucleotide sequence represented by SEQ ID NOS: 174844, 174845, 174846, 174847, 174848, 174849, 174850, 174851, 174852, 174853, 174854 were selected from the ORFs having the nucleotide sequence represented by SEQ ID NO: 173147.

The probes having the nucleotide sequence represented by SEQ ID NOS: 174855, 174856, 174857, 174858, 174859, 174860, 174861, 174862, 174863, 174864, 174865 were selected from the ORFs having the nucleotide sequence represented by SEQ ID NO: 173148.

The probes having the nucleotide sequence represented by SEQ ID NOS: 174866, 174867, 174868, 174869, 174870, 174871, 174872, 174873, 174874, 174875, 174876 were selected from the ORFs having the nucleotide sequence represented by SEQ ID NO: 173149.

The probes having the nucleotide sequence represented by SEQ ID NOS: 174877, 174878, 174879, 174880, 174881, 174882, 174883, 174884, 174885, 174886, 174887 were selected from the ORFs having the nucleotide sequence represented by SEQ ID NO: 173150.

The probes having the nucleotide sequence represented by SEQ ID NOS: 174888, 174889, 174890, 174891, 174892, 174893, 174894, 174895, 174896, 174897, 174898 were selected from the ORFs having the nucleotide sequence represented by SEQ ID NO: 173151.

The probes having the nucleotide sequence represented by SEQ ID NOS: 174899, 174900, 174901, 174902, 174903, 174904, 174905, 174906, 174907, 174908, 174909 were selected from the ORFs having the nucleotide sequence represented by SEQ ID NO: 173152.

The probes having the nucleotide sequence represented by SEQ ID NOS: 174910, 174911, 174912, 174913, 174914, 174915, 174916, 174917 were selected from the ORFs having the nucleotide sequence represented by SEQ ID NO: 173153.

The probes having the nucleotide sequence represented by SEQ ID NOS: 174918, 174919, 174920, 174921, 174922, 174923, 174924, 174925, 174926, 174927, 174928 were selected from the ORFs having the nucleotide sequence represented by SEQ ID NO: 173154.

The probes having the nucleotide sequence represented by SEQ ID NOS: 174929, 174930, 174931, 174932, 174933, 174934, 174935, 174936, 174937, 174938, 174939 were selected from the ORFs having the nucleotide sequence represented by SEQ ID NO: 173155.

The probes having the nucleotide sequence represented by SEQ ID NOS: 174940, 174941, 174942, 174943, 174944, 174945, 174946, 174947, 174948, 174949, 174950 were selected from the ORFs having the nucleotide sequence represented by SEQ ID NO: 173156.

The probes having the nucleotide sequence represented by SEQ ID NOS: 174951, 174952, 174953, 174954, 174955, 174956, 174957, 174958, 174959, 174960, 174961 were selected from the ORFs having the nucleotide sequence represented by SEQ ID NO: 173157.

The probes having the nucleotide sequence represented by SEQ ID NOS: 174962, 174963, 174964, 174965, 174966, 174967, 174968, 174969, 174970, 174971, 174972 were selected from the ORFs having the nucleotide sequence represented by SEQ ID NO: 173158.

The probes having the nucleotide sequence represented by SEQ ID NOS: 174973, 174974, 174975, 174976, 174977, 174978, 174979, 174980, 174981, 174982, 174983 were selected from the ORFs having the nucleotide sequence represented by SEQ ID NO: 173159.

The probes having the nucleotide sequence represented by SEQ ID NOS: 174984, 174985, 174986, 174987, 174988, 174989, 174990, 174991, 174992, 174993, 174994 were selected from the ORFs having the nucleotide sequence represented by SEQ ID NO: 173160.

The probes having the nucleotide sequence represented by SEQ ID NOS: 174995, 174996, 174997, 174998, 174999, 175000, 175001, 175002, 175003, 175004, 175005 were selected from the ORFs having the nucleotide sequence represented by SEQ ID NO: 173161.

The probes having the nucleotide sequence represented by SEQ ID NOS: 175006, 175007, 175008, 175009, 175010, 175011, 175012, 175013, 175014, 175015 were selected from the ORFs having the nucleotide sequence represented by SEQ ID NO: 173162.

The probes having the nucleotide sequence represented by SEQ ID NOS: 175016, 175017, 175018, 175019, 175020, 175021, 175022, 175023, 175024, 175025, 175026 were selected from the ORFs having the nucleotide sequence represented by SEQ ID NO: 173163.

The probes having the nucleotide sequence represented by SEQ ID NOS: 175027, 175028, 175029, 175030, 175031, 175032, 175033, 175034, 175035, 175036, 175037 were selected from the ORFs having the nucleotide sequence represented by SEQ ID NO: 173164.

The probes having the nucleotide sequence represented by SEQ ID NOS: 175038, 175039, 175040, 175041, 175042, 175043, 175044, 175045, 175046, 175047, 175048 were selected from the ORFs having the nucleotide sequence represented by SEQ ID NO: 173165.

The probes having the nucleotide sequence represented by SEQ ID NOS: 175049, 175050, 175051, 175052, 175053, 175054, 175055, 175056, 175057, 175058, 175059 were selected from the ORFs having the nucleotide sequence represented by SEQ ID NO: 173166.

The probes having the nucleotide sequence represented by SEQ ID NOS: 175060, 175061, 175062, 175063, 175064, 175065, 175066, 175067, 175068, 175069, 175070 were selected from the ORFs having the nucleotide sequence represented by SEQ ID NO: 173167.

The probes having the nucleotide sequence represented by SEQ ID NOS: 175071, 175072, 175073, 175074, 175075, 175076, 175077, 175078, 175079, 175080, 175081 were selected from the ORFs having the nucleotide sequence represented by SEQ ID NO: 173168.

The probes having the nucleotide sequence represented by SEQ ID NOS: 175082, 175083, 175084, 175085, 175086, 175087, 175088, 175089, 175090, 175091, 175092 were selected from the ORFs having the nucleotide sequence represented by SEQ ID NO: 173169.

The probes having the nucleotide sequence represented by SEQ ID NOS: 175093, 175094, 175095, 175096, 175097, 175098, 175099, 175100, 175101, 175102, 175103 were selected from the ORFs having the nucleotide sequence represented by SEQ ID NO: 173170.

The probes having the nucleotide sequence represented by SEQ ID NOS: 175104, 175105, 175106, 175107, 175108, 175109, 175110, 175111, 175112, 175113, 175114 were selected from the ORFs having the nucleotide sequence represented by SEQ ID NO: 173171.

The probes having the nucleotide sequence represented by SEQ ID NOS: 175115, 175116, 175117, 175118, 175119, 175120, 175121, 175122, 175123, 175124, 175125 were selected from the ORFs having the nucleotide sequence represented by SEQ ID NO: 173172.

The probes having the nucleotide sequence represented by SEQ ID NOS: 175126, 175127, 175128, 175129, 175130, 175131, 175132, 175133, 175134, 175135, 175136 were selected from the ORFs having the nucleotide sequence represented by SEQ ID NO: 173173.

The probes having the nucleotide sequence represented by SEQ ID NOS: 175137, 175138, 175139, 175140, 175141, 175142, 175143, 175144, 175145 were selected from the ORFs having the nucleotide sequence represented by SEQ ID NO: 173174.

The probes having the nucleotide sequence represented by SEQ ID NOS: 175146, 175147, 175148, 175149, 175150, 175151, 175152, 175153, 175154, 175155, 175156 were selected from the ORFs having the nucleotide sequence represented by SEQ ID NO: 173175.

The probes having the nucleotide sequence represented by SEQ ID NOS: 175157, 175158, 175159, 175160, 175161, 175162, 175163, 175164, 175165, 175166, 175167 were selected from the ORFs having the nucleotide sequence represented by SEQ ID NO: 173176.

The probes having the nucleotide sequence represented by SEQ ID NOS: 175168, 175169, 175170, 175171, 175172, 175173, 175174, 175175, 175176, 175177, 175178 were selected from the ORFs having the nucleotide sequence represented by SEQ ID NO: 173177.

The probes having the nucleotide sequence represented by SEQ ID NOS: 175179, 175180, 175181, 175182, 175183, 175184, 175185, 175186, 175187, 175188, 175189 were selected from the ORFs having the nucleotide sequence represented by SEQ ID NO: 173178.

The probes having the nucleotide sequence represented by SEQ ID NOS: 175190, 175191, 175192, 175193, 175194, 175195, 175196, 175197, 175198, 175199, 175200 were selected from the ORFs having the nucleotide sequence represented by SEQ ID NO: 173179.

The probes having the nucleotide sequence represented by SEQ ID NOS: 175201, 175202, 175203, 175204, 175205, 175206, 175207, 175208, 175209, 175210, 175211 were selected from the ORFs having the nucleotide sequence represented by SEQ ID NO: 173180.

The probes having the nucleotide sequence represented by SEQ ID NOS: 175212, 175213, 175214, 175215, 175216, 175217, 175218, 175219, 175220, 175221, 175222 were selected from the ORFs having the nucleotide sequence represented by SEQ ID NO: 173181.

The probes having the nucleotide sequence represented by SEQ ID NOS: 175223, 175224, 175225, 175226, 175227, 175228, 175229, 175230, 175231, 175232, 175233 were selected from the ORFs having the nucleotide sequence represented by SEQ ID NO: 173182.

The probes having the nucleotide sequence represented by SEQ ID NOS: 175234, 175235, 175236, 175237, 175238, 175239, 175240, 175241, 175242, 175243, 175244 were selected from the ORFs having the nucleotide sequence represented by SEQ ID NO: 173183.

The probes having the nucleotide sequence represented by SEQ ID NOS: 175245, 175246, 175247, 175248, 175249, 175250, 175251, 175252, 175253, 175254 were selected from the ORFs having the nucleotide sequence represented by SEQ ID NO: 173184.

The probes having the nucleotide sequence represented by SEQ ID NOS: 175255, 175256, 175257, 175258, 175259, 175260, 175261, 175262, 175263, 175264, 175265 were selected from the ORFs having the nucleotide sequence represented by SEQ ID NO: 173185.

The probes having the nucleotide sequence represented by SEQ ID NOS: 175266, 175267, 175268, 175269, 175270, 175271, 175272, 175273, 175274, 175275, 175276 were selected from the ORFs having the nucleotide sequence represented by SEQ ID NO: 173186.

The probes having the nucleotide sequence represented by SEQ ID NOS: 175277, 175278, 175279, 175280, 175281, 175282, 175283, 175284, 175285, 175286, 175287 were selected from the ORFs having the nucleotide sequence represented by SEQ ID NO: 173187.

The probes having the nucleotide sequence represented by SEQ ID NOS: 175288, 175289, 175290, 175291, 175292, 175293, 175294, 175295, 175296, 175297, 175298 were selected from the ORFs having the nucleotide sequence represented by SEQ ID NO: 173188.

The probes having the nucleotide sequence represented by SEQ ID NOS: 175299, 175300, 175301, 175302, 175303, 175304, 175305, 175306, 175307, 175308, 175309 were selected from the ORFs having the nucleotide sequence represented by SEQ ID NO: 173189.

The probes having the nucleotide sequence represented by SEQ ID NOS: 175310, 175311, 175312, 175313, 175314, 175315, 175316, 175317, 175318, 175319, 175320 were selected from the ORFs having the nucleotide sequence represented by SEQ ID NO: 173190.

The probes having the nucleotide sequence represented by SEQ ID NOS: 175321, 175322, 175323, 175324, 175325, 175326, 175327, 175328, 175329, 175330, 175331 were selected from the ORFs having the nucleotide sequence represented by SEQ ID NO: 173191.

The probes having the nucleotide sequence represented by SEQ ID NOS: 175332, 175333, 175334, 175335, 175336, 175337, 175338, 175339, 175340, 175341, 175342 were selected from the ORFs having the nucleotide sequence represented by SEQ ID NO: 173192.

The probes having the nucleotide sequence represented by SEQ ID NOS: 175343, 175344, 175345, 175346, 175347, 175348, 175349, 175350, 175351, 175352, 175353 were selected from the ORFs having the nucleotide sequence represented by SEQ ID NO: 173193.

The probes having the nucleotide sequence represented by SEQ ID NOS: 175354, 175355, 175356, 175357, 175358, 175359, 175360, 175361, 175362, 175363, 175364 were selected from the ORFs having the nucleotide sequence represented by SEQ ID NO: 173194.

The probes having the nucleotide sequence represented by SEQ ID NOS: 175365, 175366, 175367, 175368, 175369, 175370, 175371, 175372, 175373, 175374, 175375 were selected from the ORFs having the nucleotide sequence represented by SEQ ID NO: 173195.

The probes having the nucleotide sequence represented by SEQ ID NOS: 175376, 175377, 175378, 175379, 175380, 175381, 175382, 175383, 175384, 175385, 175386 were selected from the ORFs having the nucleotide sequence represented by SEQ ID NO: 173196.

The probes having the nucleotide sequence represented by SEQ ID NOS: 175387, 175388, 175389, 175390, 175391, 175392, 175393, 175394, 175395, 175396, 175397 were selected from the ORFs having the nucleotide sequence represented by SEQ ID NO: 173197.

The probes having the nucleotide sequence represented by SEQ ID NOS: 175398, 175399, 175400, 175401, 175402, 175403, 175404, 175405, 175406, 175407, 175408 were selected from the ORFs having the nucleotide sequence represented by SEQ ID NO: 173198.

The probes having the nucleotide sequence represented by SEQ ID NOS: 175409, 175410, 175411, 175412, 175413, 175414, 175415, 175416, 175417, 175418, 175419 were selected from the ORFs having the nucleotide sequence represented by SEQ ID NO: 173199.

The probes having the nucleotide sequence represented by SEQ ID NOS: 175420, 175421, 175422, 175423, 175424, 175425, 175426, 175427, 175428, 175429, 175430 were selected from the ORFs having the nucleotide sequence represented by SEQ ID NO: 173200.

The probes having the nucleotide sequence represented by SEQ ID NOS: 175431, 175432, 175433, 175434, 175435, 175436, 175437, 175438, 175439, 175440, 175441 were selected from the ORFs having the nucleotide sequence represented by SEQ ID NO: 173201.

The probes having the nucleotide sequence represented by SEQ ID NOS: 175442, 175443, 175444, 175445, 175446, 175447, 175448, 175449, 175450, 175451, 175452 were selected from the ORFs having the nucleotide sequence represented by SEQ ID NO: 173202.

The probes having the nucleotide sequence represented by SEQ ID NOS: 175453, 175454, 175455, 175456, 175457, 175458, 175459, 175460, 175461, 175462, 175463 were selected from the ORFs having the nucleotide sequence represented by SEQ ID NO: 173203.

The probes having the nucleotide sequence represented by SEQ ID NOS: 175464, 175465, 175466, 175467, 175468, 175469, 175470, 175471, 175472, 175473, 175474 were selected from the ORFs having the nucleotide sequence represented by SEQ ID NO: 173204.

The probes having the nucleotide sequence represented by SEQ ID NOS: 175475, 175476, 175477, 175478, 175479, 175480, 175481, 175482, 175483, 175484, 175485 were selected from the ORFs having the nucleotide sequence represented by SEQ ID NO: 173205.

The probes having the nucleotide sequence represented by SEQ ID NOS: 175486, 175487, 175488, 175489, 175490, 175491, 175492, 175493, 175494, 175495, 175496 were selected from the ORFs having the nucleotide sequence represented by SEQ ID NO: 173206.

The probes having the nucleotide sequence represented by SEQ ID NOS: 175497, 175498, 175499, 175500, 175501, 175502, 175503, 175504, 175505, 175506, 175507 were selected from the ORFs having the nucleotide sequence represented by SEQ ID NO: 173207.

The probes having the nucleotide sequence represented by SEQ ID NOS: 175508, 175509, 175510, 175511, 175512, 175513, 175514, 175515, 175516, 175517, 175518 were selected from the ORFs having the nucleotide sequence represented by SEQ ID NO: 173208.

The probes having the nucleotide sequence represented by SEQ ID NOS: 175519, 175520, 175521, 175522, 175523, 175524, 175525, 175526, 175527, 175528, 175529 were selected from the ORFs having the nucleotide sequence represented by SEQ ID NO: 173209.

The probes having the nucleotide sequence represented by SEQ ID NOS: 175530, 175531, 175532, 175533, 175534, 175535, 175536, 175537, 175538, 175539, 175540 were selected from the ORFs having the nucleotide sequence represented by SEQ ID NO: 173210.

The probes having the nucleotide sequence represented by SEQ ID NOS: 175541, 175542, 175543, 175544, 175545, 175546, 175547, 175548, 175549, 175550, 175551 were selected from the ORFs having the nucleotide sequence represented by SEQ ID NO: 173211.

The probes having the nucleotide sequence represented by SEQ ID NOS: 175552, 175553, 175554, 175555, 175556, 175557, 175558, 175559, 175560, 175561, 175562 were selected from the ORFs having the nucleotide sequence represented by SEQ ID NO: 173212.

The probes having the nucleotide sequence represented by SEQ ID NOS: 175563, 175564, 175565, 175566, 175567, 175568, 175569, 175570, 175571, 175572, 175573 were selected from the ORFs having the nucleotide sequence represented by SEQ ID NO: 173213.

The probes having the nucleotide sequence represented by SEQ ID NOS: 175574, 175575, 175576, 175577, 175578, 175579, 175580, 175581, 175582, 175583, 175584 were selected from the ORFs having the nucleotide sequence represented by SEQ ID NO: 173214.

The probes having the nucleotide sequence represented by SEQ ID NOS: 175585, 175586, 175587, 175588, 175589, 175590, 175591, 175592, 175593, 175594, 175595 were selected from the ORFs having the nucleotide sequence represented by SEQ ID NO: 173215.

The probes having the nucleotide sequence represented by SEQ ID NOS: 175596, 175597, 175598, 175599, 175600, 175601, 175602, 175603, 175604, 175605, 175606 were selected from the ORFs having the nucleotide sequence represented by SEQ ID NO: 173216.

The probes having the nucleotide sequence represented by SEQ ID NOS: 175607, 175608, 175609, 175610, 175611, 175612, 175613, 175614, 175615, 175616, 175617 were selected from the ORFs having the nucleotide sequence represented by SEQ ID NO: 173217.

The probes having the nucleotide sequence represented by SEQ ID NOS: 175618, 175619, 175620, 175621, 175622, 175623, 175624, 175625, 175626, 175627, 175628 were selected from the ORFs having the nucleotide sequence represented by SEQ ID NO: 173218.

The probes having the nucleotide sequence represented by SEQ ID NOS: 175629, 175630, 175631, 175632, 175633, 175634, 175635, 175636, 175637, 175638, 175639 were selected from the ORFs having the nucleotide sequence represented by SEQ ID NO: 173219.

The probes having the nucleotide sequence represented by SEQ ID NOS: 175640, 175641, 175642, 175643, 175644, 175645, 175646, 175647, 175648, 175649, 175650 were selected from the ORFs having the nucleotide sequence represented by SEQ ID NO: 173220.

The probes having the nucleotide sequence represented by SEQ ID NOS: 175651, 175652, 175653, 175654, 175655, 175656, 175657, 175658, 175659, 175660, 175661 were selected from the ORFs having the nucleotide sequence represented by SEQ ID NO: 173221.

The probes having the nucleotide sequence represented by SEQ ID NOS: 175662, 175663, 175664, 175665, 175666, 175667, 175668, 175669, 175670, 175671, 175672 were selected from the ORFs having the nucleotide sequence represented by SEQ ID NO: 173222.

The probes having the nucleotide sequence represented by SEQ ID NOS: 175673, 175674, 175675, 175676, 175677, 175678, 175679, 175680, 175681, 175682, 175683 were selected from the ORFs having the nucleotide sequence represented by SEQ ID NO: 173223.

The probes having the nucleotide sequence represented by SEQ ID NOS: 175684, 175685, 175686, 175687, 175688, 175689, 175690, 175691, 175692, 175693, 175694 were selected from the ORFs having the nucleotide sequence represented by SEQ ID NO: 173224.

The probes having the nucleotide sequence represented by SEQ ID NOS: 175695, 175696, 175697, 175698, 175699, 175700, 175701, 175702, 175703, 175704, 175705 were selected from the ORFs having the nucleotide sequence represented by SEQ ID NO: 173225.

The probes having the nucleotide sequence represented by SEQ ID NOS: 175706, 175707, 175708, 175709, 175710, 175711, 175712, 175713, 175714, 175715, 175716 were selected from the ORFs having the nucleotide sequence represented by SEQ ID NO: 173226.

The probes having the nucleotide sequence represented by SEQ ID NOS: 175717, 175718, 175719, 175720, 175721, 175722, 175723, 175724, 175725, 175726, 175727 were selected from the ORFs having the nucleotide sequence represented by SEQ ID NO: 173227.

The probes having the nucleotide sequence represented by SEQ ID NOS: 175728, 175729, 175730, 175731, 175732, 175733, 175734, 175735, 175736, 175737, 175738 were selected from he ORFs having the nucleotide sequence represented by SEQ ID NO: 173228.

The probes having the nucleotide sequence represented by SEQ ID NOS: 175739, 175740, 175741, 175742, 175743, 175744, 175745, 175746 were selected from the ORFs having the nucleotide sequence represented by SEQ ID NO: 173229.

The probes having the nucleotide sequence represented by SEQ ID NOS: 175747, 175748, 175749, 175750, 175751, 175752, 175753, 175754, 175755, 175756, 175757 were selected from the ORFs having the nucleotide sequence represented by SEQ ID NO: 173230.

The probes having the nucleotide sequence represented by SEQ ID NOS: 175758, 175759, 175760, 175761, 175762, 175763, 175764, 175765, 175766, 175767, 175768 were selected from the ORFs having the nucleotide sequence represented by SEQ ID NO: 173231.

The probes having the nucleotide sequence represented by SEQ ID NOS: 175769, 175770, 175771, 175772, 175773, 175774, 175775, 175776, 175777, 175778, 175779 were selected from the ORFs having the nucleotide sequence represented by SEQ ID NO: 173232.

The probes having the nucleotide sequence represented by SEQ ID NOS: 175780, 175781, 175782, 175783, 175784, 175785, 175786, 175787, 175788, 175789, 175790 were selected from the ORFs having the nucleotide sequence represented by SEQ ID NO: 173233.

The probes having the nucleotide sequence represented by SEQ ID NOS: 175791, 175792, 175793, 175794, 175795, 175796, 175797, 175798, 175799, 175800, 175801 were selected from the ORFs having the nucleotide sequence represented by SEQ ID NO: 173234.

The probes having the nucleotide sequence represented by SEQ ID NOS: 175802, 175803, 175804, 175805, 175806, 175807, 175808, 175809, 175810, 175811, 175812 were selected from the ORFs having the nucleotide sequence represented by SEQ ID NO: 173235.

The probes having the nucleotide sequence represented by SEQ ID NOS: 175813, 175814, 175815, 175816, 175817, 175818, 175819, 175820, 175821, 175822, 175823 were selected from the ORFs having the nucleotide sequence represented by SEQ ID NO: 173236.

The probes having the nucleotide sequence represented by SEQ ID NOS: 175824, 175825, 175826, 175827, 175828, 175829, 175830, 175831, 175832, 175833, 175834 were selected from the ORFs having the nucleotide sequence represented by SEQ ID NO: 173237.

The probes having the nucleotide sequence represented by SEQ ID NOS: 175835, 175836, 175837, 175838, 175839, 175840, 175841, 175842, 175843, 175844, 175845 were selected from the ORFs having the nucleotide sequence represented by SEQ ID NO: 173238.

The probes having the nucleotide sequence represented by SEQ ID NOS: 175846, 175847, 175848, 175849, 175850, 175851, 175852, 175853, 175854, 175855, 175856 were selected from the ORFs having the nucleotide sequence represented by SEQ ID NO: 173239.

The probes having the nucleotide sequence represented by SEQ ID NOS: 175857, 175858, 175859, 175860, 175861, 175862, 175863, 175864, 175865, 175866, 175867 were selected from the ORFs having the nucleotide sequence represented by SEQ ID NO: 173240.

The probes having the nucleotide sequence represented by SEQ ID NOS: 175868, 175869, 175870, 175871, 175872, 175873, 175874, 175875, 175876, 175877, 175878 were selected from the ORFs having the nucleotide sequence represented by SEQ ID NO: 173241.

The probes having the nucleotide sequence represented by SEQ ID NOS: 175879, 175880, 175881, 175882, 175883, 175884, 175885, 175886, 175887, 175888, 175889 were selected from the ORFs having the nucleotide sequence represented by SEQ ID NO: 173242.

The probes having the nucleotide sequence represented by SEQ ID NOS: 175890, 175891, 175892, 175893, 175894, 175895, 175896, 175897, 175898, 175899, 175900 were selected from the ORFs having the nucleotide sequence represented by SEQ ID NO: 173243.

The probes having the nucleotide sequence represented by SEQ ID NOS: 175901, 175902, 175903, 175904, 175905, 175906, 175907, 175908, 175909, 175910, 175911 were selected from he ORFs having the nucleotide sequence represented by SEQ D NO: 173244.

The probes having the nucleotide sequence represented by SEQ ID NOS: 175912, 175913, 175914, 175915, 175916, 175917, 175918, 175919, 175920, 175921, 175922 were selected from the ORFs having the nucleotide sequence represented by SEQ ID NO: 173245.

The probes having the nucleotide sequence represented by SEQ ID NOS: 175923, 175924, 175925, 175926, 175927, 175928, 175929, 175930, 175931, 175932 were selected from the ORFs having the nucleotide sequence represented by SEQ ID NO: 173246.

The probes having the nucleotide sequence represented by SEQ ID NOS: 175933, 175934, 175935, 175936, 175937, 175938, 175939, 175940, 175941, 175942, 175943 were selected from the ORFs having the nucleotide sequence represented by SEQ ID NO: 173247.

The probes having the nucleotide sequence represented by SEQ ID NOS: 175944, 175945, 175946, 175947, 175948, 175949, 175950, 175951, 175952, 175953, 175954 were selected from the ORFs having the nucleotide sequence represented by SEQ ID NO: 173248.

The probes having the nucleotide sequence represented by SEQ ID NOS: 175955, 175956, 175957, 175958, 175959, 175960, 175961, 175962, 175963, 175964, 175965 were selected from the ORFs having the nucleotide sequence represented by SEQ ID NO: 173249.

The probes having the nucleotide sequence represented by SEQ ID NOS: 175966, 175967, 175968, 175969, 175970, 175971, 175972, 175973, 175974, 175975, 175976 were selected from the ORFs having the nucleotide sequence represented by SEQ ID NO: 173250.

The probes having the nucleotide sequence represented by SEQ ID NOS: 175977, 175978, 175979, 175980, 175981, 175982, 175983, 175984, 175985, 175986, 175987 were selected from the ORFs having the nucleotide sequence represented by SEQ ID NO: 173251.

The probes having the nucleotide sequence represented by SEQ ID NOS: 175988, 175989, 175990, 175991, 175992, 175993, 175994, 175995, 175996, 175997, 175998 were selected from the ORFs having the nucleotide sequence represented by SEQ ID NO: 173252.

The probes having the nucleotide sequence represented by SEQ ID NOS: 175999, 176000, 176001, 176002, 176003, 176004, 176005, 176006, 176007, 176008, 176009 were selected from the ORFs having the nucleotide sequence represented by SEQ ID NO: 173253.

The probes having the nucleotide sequence represented by SEQ ID NOS: 176010, 176011, 176012, 176013, 176014, 176015, 176016, 176017, 176018, 176019, 176020 were selected from the ORFs having the nucleotide sequence represented by SEQ ID NO: 173254.

The probes having the nucleotide sequence represented by SEQ ID NOS: 176021, 176022, 176023, 176024, 176025, 176026, 176027, 176028, 176029, 176030, 176031 were selected from the ORFs having the nucleotide sequence represented by SEQ ID NO: 173255.

The probes having the nucleotide sequence represented by SEQ ID NOS: 176032, 176033, 176034, 176035, 176036, 176037, 176038, 176039, 176040, 176041, 176042 were selected from the ORFs having the nucleotide sequence represented by SEQ ID NO: 173256.

The probes having the nucleotide sequence represented by SEQ ID NOS: 176043, 176044, 176045, 176046, 176047, 176048, 176049, 176050, 176051, 176052, 176053 were selected from the ORFs having the nucleotide sequence represented by SEQ ID NO: 173257.

The probes having the nucleotide sequence represented by SEQ ID NOS: 176054, 176055, 176056, 176057, 176058, 176059, 176060, 176061, 176062, 176063, 176064 were selected from the ORFs having the nucleotide sequence represented by SEQ ID NO: 173258.

The probes having the nucleotide sequence represented by SEQ ID NOS: 176065, 176066, 176067, 176068, 176069, 176070, 176071, 176072, 176073, 176074, 176075 were selected from the ORFs having the nucleotide sequence represented by SEQ ID NO: 173259.

The probes having the nucleotide sequence represented by SEQ ID NOS: 176076, 176077, 176078, 176079, 176080, 176081, 176082, 176083, 176084, 176085, 176086 were selected from the ORFs having the nucleotide sequence represented by SEQ ID NO: 173260.

The probes having the nucleotide sequence represented by SEQ ID NOS: 176087, 176088, 176089, 176090, 176091, 176092, 176093, 176094, 176095, 176096, 176097 were selected from the ORFs having the nucleotide sequence represented by SEQ ID NO: 173261.

The probes having the nucleotide sequence represented by SEQ ID NOS: 176098, 176099, 176100, 176101, 176102, 176103, 176104, 176105, 176106, 176107, 176108 were selected from the ORFs having the nucleotide sequence represented by SEQ ID NO: 173262.

The probes having the nucleotide sequence represented by SEQ ID NOS: 176109, 176110, 176111, 176112, 176113, 176114, 176115, 176116, 176117, 176118, 176119 were selected from the ORFs having the nucleotide sequence represented by SEQ ID NO: 173263.

The probes having the nucleotide sequence represented by SEQ ID NOS: 176120, 176121, 176122, 176123, 176124, 176125, 176126, 176127, 176128, 176129, 176130 were selected from the ORFs having the nucleotide sequence represented by SEQ ID NO: 173264.

The probes having the nucleotide sequence represented by SEQ ID NOS: 176131, 176132, 176133, 176134, 176135, 176136, 176137, 176138, 176139, 176140, 176141 were selected from the ORFs having the nucleotide sequence represented by SEQ ID NO: 173265.

The probes having the nucleotide sequence represented by SEQ ID NOS: 176142, 176143, 176144, 176145, 176146, 176147, 176148, 176149, 176150, 176151, 176152 were selected from the ORFs having the nucleotide sequence represented by SEQ ID NO: 173266.

The probes having the nucleotide sequence represented by SEQ ID NOS: 176153, 176154, 176155, 176156, 176157, 176158, 176159, 176160, 176161, 176162, 176163 were selected from the ORFs having the nucleotide sequence represented by SEQ ID NO: 173267.

The probes having the nucleotide sequence represented by SEQ ID NOS: 176164, 176165, 176166, 176167, 176168, 176169, 176170, 176171, 176172, 176173, 176174 were selected from the ORFs having the nucleotide sequence represented by SEQ ID NO: 173268.

The probes having the nucleotide sequence represented by SEQ ID NOS: 176175, 176176, 176177, 176178, 176179, 176180, 176181, 176182, 176183, 176184, 176185 were selected from the ORFs having the nucleotide sequence represented by SEQ ID NO: 173269.

The probes having the nucleotide sequence represented by SEQ ID NOS: 176186, 176187, 176188, 176189, 176190, 176191, 176192, 176193, 176194, 176195, 176196 were selected from the ORFs having the nucleotide sequence represented by SEQ ID NO: 173270.

The probes having the nucleotide sequence represented by SEQ ID NOS: 176197, 176198, 176199, 176200, 176201, 176202, 176203, 176204, 176205, 176206, 176207 were selected from the ORFs having the nucleotide sequence represented by SEQ ID NO: 173271.

The probes having the nucleotide sequence represented by SEQ ID NOS: 176208, 176209, 176210, 176211, 176212, 176213, 176214, 176215, 176216, 176217, 176218 were selected from the ORFs having the nucleotide sequence represented by SEQ ID NO: 173272.

The probes having the nucleotide sequence represented by SEQ ID NOS: 176219, 176220, 176221, 176222, 176223, 176224, 176225, 176226, 176227, 176228, 176229 were selected from the ORFs having the nucleotide sequence represented by SEQ ID NO: 173273.

The probes having the nucleotide sequence represented by SEQ ID NOS: 176230, 176231, 176232, 176233, 176234, 176235, 176236, 176237, 176238, 176239, 176240 were selected from the ORFs having the nucleotide sequence represented by SEQ ID NO: 173274.

The probes having the nucleotide sequence represented by SEQ ID NOS: 176241, 176242, 176243, 176244, 176245, 176246, 176247, 176248, 176249, 176250, 176251 were selected from the ORFs having the nucleotide sequence represented by SEQ ID NO: 173275.

The probes having the nucleotide sequence represented by SEQ ID NOS: 176252, 176253, 176254, 176255, 176256, 176257, 176258, 176259, 176260, 176261, 176262 were selected from the ORFs having the nucleotide sequence represented by SEQ ID NO: 173276.

The probes having the nucleotide sequence represented by SEQ ID NOS: 176263, 176264, 176265, 176266, 176267, 176268, 176269, 176270, 176271, 176272, 176273 were selected from the ORFs having the nucleotide sequence represented by SEQ ID NO: 173277.

The probes having the nucleotide sequence represented by SEQ ID NOS: 176274, 176275, 176276, 176277, 176278, 176279, 176280, 176281, 176282, 176283, 176284 were selected from the ORFs having the nucleotide sequence represented by SEQ ID NO: 173278.

The probes having the nucleotide sequence represented by SEQ ID NOS: 176285, 176286, 176287, 176288, 176289, 176290, 176291, 176292, 176293, 176294, 176295 were selected from the ORFs having the nucleotide sequence represented by SEQ ID NO: 173279.

The probes having the nucleotide sequence represented by SEQ ID NOS: 176296, 176297, 176298, 176299, 176300, 176301, 176302, 176303, 176304, 176305, 176306 were selected from the ORFs having the nucleotide sequence represented by SEQ ID NO: 173280.

The probes having the nucleotide sequence represented by SEQ ID NOS: 176307, 176308, 176309, 176310, 176311, 176312, 176313, 176314, 176315, 176316, 176317 were selected from the ORFs having the nucleotide sequence represented by SEQ ID NO: 173281.

The probes having the nucleotide sequence represented by SEQ ID NOS: 176318, 176319, 176320, 176321, 176322, 176323, 176324, 176325, 176326, 176327, 176328 were selected from the ORFs having the nucleotide sequence represented by SEQ ID NO: 173282.

The probes having the nucleotide sequence represented by SEQ ID NOS: 176329, 176330, 176331, 176332, 176333, 176334, 176335, 176336, 176337, 176338, 176339 were selected from the ORFs having the nucleotide sequence represented by SEQ ID NO: 173283.

The probes having the nucleotide sequence represented by SEQ ID NOS: 176340, 176341, 176342, 176343, 176344, 176345, 176346, 176347, 176348, 176349, 176350 were selected from the ORFs having the nucleotide sequence represented by SEQ ID NO: 173284.

The probes having the nucleotide sequence represented by SEQ ID NOS: 176351, 176352, 176353, 176354, 176355, 176356, 176357, 176358, 176359, 176360, 176361 were selected from the ORFs having the nucleotide sequence represented by SEQ ID NO: 173285.

The probes having the nucleotide sequence represented by SEQ ID NOS: 176362, 176363, 176364, 176365, 176366, 176367, 176368, 176369, 176370, 176371, 176372 were selected from the ORFs having the nucleotide sequence represented by SEQ ID NO: 173286.

The probes having the nucleotide sequence represented by SEQ ID NOS: 176373, 176374, 176375, 176376, 176377, 176378, 176379, 176380, 176381, 176382, 176383 were selected from the ORFs having the nucleotide sequence represented by SEQ ID NO: 173287.

The probes having the nucleotide sequence represented by SEQ ID NOS: 176384, 176385, 176386, 176387, 176388, 176389, 176390, 176391, 176392, 176393, 176394 were selected from the ORFs having the nucleotide sequence represented by SEQ ID NO: 173288.

The probes having the nucleotide sequence represented by SEQ ID NOS: 176395, 176396, 176397, 176398, 176399, 176400, 176401, 176402, 176403, 176404, 176405 were selected from the ORFs having the nucleotide sequence represented by SEQ ID NO: 173289.

The probes having the nucleotide sequence represented by SEQ ID NOS: 176406, 176407, 176408, 176409, 176410, 176411, 176412, 176413 were selected from the ORFs having the nucleotide sequence represented by SEQ ID NO: 173290.

The probes having the nucleotide sequence represented by SEQ ID NOS: 176414, 176415, 176416, 176417, 176418, 176419, 176420, 176421, 176422, 176423, 176424 were selected from the ORFs having the nucleotide sequence represented by SEQ ID NO: 173291.

The probes having the nucleotide sequence represented by SEQ ID NOS: 176425, 176426, 176427, 176428, 176429, 176430, 176431, 176432, 176433, 176434, 176435 were selected from the ORFs having the nucleotide sequence represented by SEQ ID NO: 173292.

The probes having the nucleotide sequence represented by SEQ ID NOS: 176436, 176437, 176438, 176439, 176440, 176441, 176442, 176443, 176444, 176445, 176446 were selected from the ORFs having the nucleotide sequence represented by SEQ ID NO: 173293.

The probes having the nucleotide sequence represented by SEQ ID NOS: 176447, 176448, 176449, 176450, 176451, 176452, 176453, 176454, 176455, 176456, 176457 were selected from the ORFs having the nucleotide sequence represented by SEQ ID NO: 173294.

The probes having the nucleotide sequence represented by SEQ ID NOS: 176458, 176459, 176460, 176461, 176462, 176463, 176464, 176465, 176466, 176467, 176468 were selected from the ORFs having the nucleotide sequence represented by SEQ ID NO: 173295.

The probes having the nucleotide sequence represented by SEQ ID NOS: 176469, 176470, 176471, 176472, 176473, 176474, 176475, 176476, 176477, 176478, 176479 were selected from the ORFs having the nucleotide sequence represented by SEQ ID NO: 173296.

The probes having the nucleotide sequence represented by SEQ ID NOS: 176480, 176481, 176482, 176483, 176484, 176485, 176486, 176487, 176488, 176489, 176490 were selected from the ORFs having the nucleotide sequence represented by SEQ ID NO: 173297.

The probes having the nucleotide sequence represented by SEQ ID NOS: 176491, 176492, 176493, 176494, 176495, 176496, 176497, 176498, 176499, 176500, 176501 were selected from the ORFs having the nucleotide sequence represented by SEQ ID NO: 173298.

The probes having the nucleotide sequence represented by SEQ ID NOS: 176502, 176503, 176504, 176505, 176506, 176507, 176508, 176509, 176510, 176511, 176512 were selected from the ORFs having the nucleotide sequence represented by SEQ ID NO: 173299.

The probes having the nucleotide sequence represented by SEQ ID NOS: 176513, 176514, 176515, 176516, 176517, 176518, 176519, 176520, 176521, 176522, 176523 were selected from the ORFs having the nucleotide sequence represented by SEQ ID NO: 173300.

The probes having the nucleotide sequence represented by SEQ ID NOS: 176524, 176525, 176526, 176527, 176528, 176529, 176530, 176531, 176532, 176533, 176534 were selected from the ORFs having the nucleotide sequence represented by SEQ ID NO: 173301.

The probes having the nucleotide sequence represented by SEQ ID NOS: 176535, 176536, 176537, 176538, 176539, 176540, 176541, 176542, 176543, 176544, 176545 were selected from the ORFs having the nucleotide sequence represented by SEQ ID NO: 173302.

The probes having the nucleotide sequence represented by SEQ ID NOS: 176546, 176547, 176548, 176549, 176550, 176551, 176552, 176553, 176554, 176555, 176556 were selected from the ORFs having the nucleotide sequence represented by SEQ ID NO: 173303.

The probes having the nucleotide sequence represented by SEQ ID NOS: 176557, 176558, 176559, 176560, 176561, 176562, 176563, 176564, 176565, 176566, 176567 were selected from the ORFs having the nucleotide sequence represented by SEQ ID NO: 173304.

The probes having the nucleotide sequence represented by SEQ ID NOS: 176568, 176569, 176570, 176571, 176572, 176573, 176574, 176575, 176576, 176577, 176578 were selected from the ORFs having the nucleotide sequence represented by SEQ ID NO: 173305.

The probes having the nucleotide sequence represented by SEQ ID NOS: 176579, 176580, 176581, 176582, 176583, 176584, 176585, 176586, 176587, 176588, 176589 were selected from the ORFs having the nucleotide sequence represented by SEQ ID NO: 173306.

The probes having the nucleotide sequence represented by SEQ ID NOS: 176590, 176591, 176592, 176593, 176594, 176595, 176596, 176597, 176598, 176599, 176600 were selected from the ORFs having the nucleotide sequence represented by SEQ ID NO: 173307.

The probes having the nucleotide sequence represented by SEQ ID NOS: 176601, 176602, 176603, 176604, 176605, 176606, 176607, 176608 were selected from the ORFs having the nucleotide sequence represented by SEQ ID NO: 173308.

The probes having the nucleotide sequence represented by SEQ ID NOS: 176609, 176610, 176611, 176612, 176613, 176614, 176615, 176616, 176617, 176618, 176619 were selected from the ORFs having the nucleotide sequence represented by SEQ ID NO: 173309.

The probes having the nucleotide sequence represented by SEQ ID NOS: 176620, 176621, 176622, 176623, 176624, 176625, 176626, 176627, 176628, 176629, 176630 were selected from the ORFs having the nucleotide sequence represented by SEQ ID NO: 173310.

The probes having the nucleotide sequence represented by SEQ ID NOS: 176631, 176632, 176633, 176634, 176635, 176636, 176637, 176638, 176639, 176640, 176641 were selected from the ORFs having the nucleotide sequence represented by SEQ ID NO: 173311.

The probes having the nucleotide sequence represented by SEQ ID NOS: 176642, 176643, 176644, 176645, 176646, 176647, 176648, 176649, 176650, 176651, 176652 were selected from the ORFs having the nucleotide sequence represented by SEQ ID NO: 173312.

The probes having the nucleotide sequence represented by SEQ ID NOS: 176653, 176654, 176655, 176656, 176657, 176658, 176659, 176660, 176661, 176662, 176663 were selected from the ORFs having the nucleotide sequence represented by SEQ ID NO: 173313.

The probes having the nucleotide sequence represented by SEQ ID NOS: 176664, 176665, 176666, 176667, 176668, 176669, 176670, 176671, 176672, 176673, 176674 were selected from the ORFs having the nucleotide sequence represented by SEQ ID NO: 173314.

The probes having the nucleotide sequence represented by SEQ ID NOS: 176675, 176676, 176677, 176678, 176679, 176680, 176681, 176682, 176683, 176684, 176685 were selected from the ORFs having the nucleotide sequence represented by SEQ ID NO: 173315.

The probes having the nucleotide sequence represented by SEQ ID NOS: 176686, 176687, 176688, 176689, 176690, 176691, 176692, 176693, 176694, 176695, 176696 were selected from the ORFs having the nucleotide sequence represented by SEQ ID NO: 173316.

The probes having the nucleotide sequence represented by SEQ ID NOS: 176697, 176698, 176699, 176700, 176701, 176702, 176703, 176704, 176705, 176706, 176707 were selected from the ORFs having the nucleotide sequence represented by SEQ ID NO: 173317.

The probes having the nucleotide sequence represented by SEQ ID NOS: 176708, 176709, 176710, 176711, 176712, 176713, 176714, 176715, 176716, 176717, 176718 were selected from the ORFs having the nucleotide sequence represented by SEQ ID NO: 173318.

The probes having the nucleotide sequence represented by SEQ ID NOS: 176719, 176720, 176721, 176722, 176723, 176724, 176725, 176726, 176727, 176728, 176729 were selected from the ORFs having the nucleotide sequence represented by SEQ ID NO: 173319.

The probes having the nucleotide sequence represented by SEQ ID NOS: 176730, 176731, 176732, 176733, 176734, 176735, 176736, 176737, 176738, 176739, 176740 were selected from the ORFs having the nucleotide sequence represented by SEQ ID NO: 173320.

The probes having the nucleotide sequence represented by SEQ ID NOS: 176741, 176742, 176743, 176744, 176745, 176746, 176747, 176748, 176749, 176750, 176751 were selected from the ORFs having the nucleotide sequence represented by SEQ ID NO: 173321.

The probes having the nucleotide sequence represented by SEQ ID NOS: 176752, 176753, 176754, 176755, 176756, 176757, 176758, 176759, 176760, 176761, 176762 were selected from the ORFs having the nucleotide sequence represented by SEQ ID NO: 173322.

The probes having the nucleotide sequence represented by SEQ ID NOS: 176763, 176764, 176765, 176766, 176767, 176768, 176769, 176770, 176771, 176772, 176773 were selected from the ORFs having the nucleotide sequence represented by SEQ ID NO: 173323.

The probes having the nucleotide sequence represented by SEQ ID NOS: 176774, 176775, 176776, 176777, 176778, 176779, 176780, 176781, 176782, 176783, 176784 were selected from the ORFs having the nucleotide sequence represented by SEQ ID NO: 173324.

The probes having the nucleotide sequence represented by SEQ ID NOS: 176785, 176786, 176787, 176788, 176789, 176790, 176791, 176792, 176793, 176794, 176795 were selected from the ORFs having the nucleotide sequence represented by SEQ ID NO: 173325.

The probes having the nucleotide sequence represented by SEQ ID NOS: 176796, 176797, 176798, 176799, 176800, 176801, 176802, 176803, 176804, 176805, 176806 were selected from the ORFs having the nucleotide sequence represented by SEQ ID NO: 173326.

The probes having the nucleotide sequence represented by SEQ ID NOS: 176807, 176808, 176809, 176810, 176811, 176812, 176813, 176814, 176815, 176816, 176817 were selected from the ORFs having the nucleotide sequence represented by SEQ ID NO: 173327.

The probes having the nucleotide sequence represented by SEQ ID NOS: 176818, 176819, 176820, 176821, 176822, 176823, 176824, 176825, 176826, 176827, 176828 were selected from the ORFs having the nucleotide sequence represented by SEQ ID NO: 173328.

The probes having the nucleotide sequence represented by SEQ ID NOS: 176829, 176830, 176831, 176832, 176833, 176834, 176835, 176836, 176837, 176838, 176839 were selected from the ORFs having the nucleotide sequence represented by SEQ ID NO: 173329.

The probes having the nucleotide sequence represented by SEQ ID NOS: 176840, 176841, 176842, 176843, 176844, 176845, 176846, 176847, 176848, 176849, 176850 were selected from the ORFs having the nucleotide sequence represented by SEQ ID NO: 173330.

The probes having the nucleotide sequence represented by SEQ ID NOS: 176851, 176852, 176853, 176854, 176855, 176856, 176857, 176858, 176859, 176860, 176861 were selected from the ORFs having the nucleotide sequence represented by SEQ ID NO: 173331.

The probes having the nucleotide sequence represented by SEQ ID NOS: 176862, 176863, 176864, 176865, 176866, 176867, 176868, 176869, 176870, 176871, 176872 were selected from the ORFs having the nucleotide sequence represented by SEQ ID NO: 173332.

The probes having the nucleotide sequence represented by SEQ ID NOS: 176873, 176874, 176875, 176876, 176877, 176878, 176879, 176880, 176881, 176882, 176883 were selected from the ORFs having the nucleotide sequence represented by SEQ ID NO: 173333.

The probes having the nucleotide sequence represented by SEQ ID NOS: 176884, 176885, 176886, 176887, 176888, 176889, 176890, 176891, 176892, 176893, 176894 were selected from the ORFs having the nucleotide sequence represented by SEQ ID NO: 173334.

The probes having the nucleotide sequence represented by SEQ ID NOS: 176895, 176896, 176897, 176898, 176899, 176900, 176901, 176902., 176903, 176904, 176905 were selected from the ORFs having the nucleotide sequence represented by SEQ ID NO: 173335.

The probes having the nucleotide sequence represented by SEQ ID NOS: 176906, 176907, 176908, 176909, 176910, 176911, 176912, 176913, 176914, 176915, 176916 were selected from the ORFs having the nucleotide sequence represented by SEQ ID NO: 173336.

The probes having the nucleotide sequence represented by SEQ ID NOS: 176917, 176918, 176919, 176920, 176921, 176922, 176923, 176924 were selected from the ORFs having the nucleotide sequence represented by SEQ ID NO: 173337.

The probes having the nucleotide sequence represented by SEQ ID NOS: 176925, 176926, 176927, 176928, 176929, 176930, 176931, 176932, 176933, 176934, 176935 were selected from the ORFs having the nucleotide sequence represented by SEQ ID NO: 173338.

The probes having the nucleotide sequence represented by SEQ ID NOS: 176936, 176937, 176938, 176939, 176940, 176941, 176942, 176943, 176944, 176945, 176946 were selected from the ORFs having the nucleotide sequence represented by SEQ ID NO: 173339.

The probes having the nucleotide sequence represented by SEQ ID NOS: 176947, 176948, 176949, 176950, 176951, 176952, 176953, 176954, 176955, 176956, 176957 were selected from the ORFs having the nucleotide sequence represented by SEQ ID NO: 173340.

The probes having the,nucleotide sequence represented by SEQ ID NOS: 176958, 176959, 176960, 176961, 176962, 176963, 176964, 176965, 176966, 176967, 176968 were selected from the ORFs having the nucleotide sequence represented by SEQ ID NO: 173341.

The probes having the nucleotide sequence represented by SEQ ID NOS: 176969, 176970, 176971, 176972, 176973, 176974, 176975, 176976, 176977, 176978, 176979 were selected from the ORFs having the nucleotide sequence represented by SEQ ID NO: 173342.

The probes having the nucleotide sequence represented by SEQ ID NOS: 176980, 176981, 176982, 176983, 176984, 176985, 176986, 176987, 176988, 176989, 176990 were selected from the ORFs having the nucleotide sequence represented by SEQ ID NO: 173343.

The probes having the nucleotide sequence represented by SEQ ID NOS: 176991, 176992, 176993, 176994, 176995, 176996, 176997, 176998, 176999, 177000, 177001 were selected from the ORFs having the nucleotide sequence represented by SEQ ID NO: 173344.

The probes having the nucleotide sequence represented by SEQ ID NOS: 177002, 177003, 177004, 177005, 177006, 177007, 177008, 177009, 177010, 177011, 177012 were selected from the ORFs having the nucleotide sequence represented by SEQ ID NO: 173345.

The probes having the nucleotide sequence represented by SEQ ID NOS: 177013, 177014, 177015, 177016, 177017, 177018, 177019, 177020, 177021, 177022, 177023 were selected from the ORFs having the nucleotide sequence represented by SEQ ID NO: 173346.

The probes having the nucleotide sequence represented by SEQ ID NOS: 177024, 177025, 177026, 177027, 177028, 177029, 177030, 177031, 177032, 177033, 177034 were selected from the ORFs having the nucleotide sequence represented by SEQ ID NO: 173347.

The probes having the nucleotide sequence represented by SEQ ID NOS: 177035, 177036, 177037, 177038, 177039, 177040, 177041, 177042, 177043, 177044, 177045 were selected from the ORFs having the nucleotide sequence represented by SEQ ID NO: 173348.

The probes having the nucleotide sequence represented by SEQ ID NOS: 177046, 177047, 177048, 177049, 177050, 177051, 177052, 177053, 177054, 177055, 177056 were selected from the ORFs having the nucleotide sequence represented by SEQ ID NO: 173349.

The probes having the nucleotide sequence represented by SEQ ID NOS: 177057, 177058, 177059, 177060, 177061, 177062, 177063, 177064, 177065, 177066, 177067 were selected from the ORFs having the nucleotide sequence represented by SEQ ID NO: 173350.

The probes having the nucleotide sequence represented by SEQ ID NOS: 177068, 177069, 177070, 177071, 177072, 177073, 177074, 177075, 177076, 177077, 177078 were selected from the ORFs having the nucleotide sequence represented by SEQ ID NO: 173351.

The probes having the nucleotide sequence represented by SEQ ID NOS: 177079, 177080, 177081, 177082, 177083, 177084, 177085, 177086, 177087, 177088, 177089 were selected from the ORFs having the nucleotide sequence represented by SEQ ID NO: 173352.

The probes having the nucleotide sequence represented by SEQ ID NOS: 177090, 177091, 177092, 177093, 177094, 177095, 177096, 177097, 177098, 177099, 177100 were selected from the ORFs having the nucleotide sequence represented by SEQ ID NO: 173353.

The probes having the nucleotide sequence represented by SEQ ID NOS: 177101, 177102, 177103, 177104, 177105, 177106, 177107, 177108, 177109, 177110, 177111 were selected from the ORFs having the nucleotide sequence represented by SEQ ID NO: 173354.

The probes having the nucleotide sequence represented by SEQ ID NOS: 177112, 177113, 177114, 177115, 177116, 177117, 177118, 177119, 177120, 177121, 177122 were selected from the ORFs having the nucleotide sequence represented by SEQ ID NO: 173355.

The probes having the nucleotide sequence represented by SEQ ID NOS: 177123, 177124, 177125, 177126, 177127, 177128, 177129, 177130, 177131, 177132, 177133 were selected from the ORFs having the nucleotide sequence represented by SEQ ID NO: 173356.

The probes having the nucleotide sequence represented by SEQ ID NOS: 177134, 177135, 177136, 177137, 177138, 177139, 177140, 177141, 177142, 177143, 177144 were selected from the ORFs having the nucleotide sequence represented by SEQ ID NO: 13357.

The probes having the nucleotide sequence represented by SEQ ID NOS: 177145, 177146, 177147, 177148, 177149, 177150, 177151, 177152, 177153, 177154, 177155 were selected from the ORFs having the nucleotide sequence represented by SEQ ID NO: 173358.

The probes having the nucleotide sequence represented by SEQ ID NOS: 177156, 177157, 177158, 177159, 177160, 177161, 177162, 177163, 177164, 177165, 177166 were selected from the ORFs having the nucleotide sequence represented by SEQ ID NO: 173359.

The probes having the nucleotide sequence represented by SEQ ID NOS: 177167, 177168, 177169, 177170, 177171, 177172, 177173, 177174, 177175, 177176, 177177 were selected from the ORFs having the nucleotide sequence represented by SEQ ID NO: 173360.

The probes having the nucleotide sequence represented by SEQ ID NOS: 177178, 177179, 177180, 177181, 177182, 177183, 177184, 177185, 177186, 177187, 177188 were selected from the ORFs having the nucleotide sequence represented by SEQ ID NO: 173361.

The probes having the nucleotide sequence represented by SEQ ID NOS: 177189, 177190, 177191, 177192, 177193, 177194, 177195, 177196, 177197, 177198, 177199 were selected from the ORFs having the nucleotide sequence represented by SEQ ID NO: 173362.

The probes having the nucleotide sequence represented by SEQ ID NOS: 177200, 177201, 177202, 177203, 177204, 177205, 177206, 177207, 177208, 177209, 177210 were selected from the ORFs having the nucleotide sequence represented by SEQ ID NO: 173363.

The probes having the nucleotide sequence represented by SEQ ID NOS: 177211, 177212, 177213, 177214, 177215, 177216, 177217, 177218, 177219, 177220, 177221 were selected from the ORFs having the nucleotide sequence represented by SEQ ID NO: 173364.

The probes having the nucleotide sequence represented by SEQ ID NOS: 177222, 177223, 177224, 177225, 177226, 177227, 177228, 177229, 177230, 177231, 177232 were selected from the ORFs having the nucleotide sequence represented by SEQ ID NO: 173365.

The probes having the nucleotide sequence represented by SEQ ID NOS: 177233, 177234, 177235, 177236, 177237, 177238, 177239, 177240, 177241, 177242, 177243 were selected from the ORFs having the nucleotide sequence represented by SEQ ID NO: 173366.

The probes having the nucleotide sequence represented by SEQ ID NOS: 177244, 177245, 177246, 177247, 177248, 177249, 177250, 177251, 177252, 177253, 177254 were selected from the ORFs having the nucleotide sequence represented by SEQ ID NO: 173367.

The probes having the nucleotide sequence represented by SEQ ID NOS: 177255, 177256, 177257, 177258, 177259, 177260, 177261, 177262, 177263, 177264, 177265 were selected from the ORFs having the nucleotide sequence represented by SEQ ID NO: 173368.

The probes having the nucleotide sequence represented by SEQ ID NOS: 177266, 177267, 177268, 177269, 177270, 177271, 177272, 177273, 177274, 177275, 177276 were selected from the ORFs having the nucleotide sequence represented by SEQ ID NO: 173369.

The probes having the nucleotide sequence represented by SEQ ID NOS: 177277, 177278, 177279, 177280, 177281, 177282, 177283, 177284, 177285, 177286, 177287 were selected from the ORFs having the nucleotide sequence represented by SEQ ID NO: 173370.

The probes having the nucleotide sequence represented by SEQ ID NOS: 177288, 177289, 177290, 177291, 177292, 177293, 177294, 177295, 177296, 177297, 177298 were selected from the ORFs having the nucleotide sequence represented by SEQ ID NO: 173371.

The probes having the nucleotide sequence represented by SEQ ID NOS: 177299, 177300, 177301, 177302, 177303, 177304, 177305, 177306, 177307, 177308, 177309 were selected from the ORFs having the nucleotide sequence represented by SEQ ID NO: 173372.

The probes having the nucleotide sequence represented by SEQ ID NOS: 177310, 177311, 177312, 177313, 177314, 177315, 177316, 177317, 177318, 177319, 177320 were selected from the ORFs having the nucleotide sequence represented by SEQ ID NO: 173373.

The probes having the nucleotide sequence represented by SEQ ID NOS: 177321, 177322, 177323, 177324, 177325, 177326, 177327, 177328, 177329, 177330, 177331 were selected from the ORFs having the nucleotide sequence represented by SEQ ID NO: 173374.

The probes having the nucleotide sequence represented by SEQ ID NOS: 177332, 177333, 177334, 177335, 177336, 177337, 177338, 177339, 177340, 177341, 177342 were selected from the ORFs having the nucleotide sequence represented by SEQ ID NO: 173375.

The probes having the nucleotide sequence represented by SEQ ID NOS: 177343, 177344, 177345, 177346, 177347, 177348, 177349, 177350, 177351, 177352, 177353 were selected from the ORFs having the nucleotide sequence represented by SEQ ID NO: 173376.

The probes having the nucleotide sequence represented by SEQ ID NOS: 177354, 177355, 177356, 177357, 177358, 177359, 177360, 177361, 177362, 177363, 177364 were selected from the ORFs having the nucleotide sequence represented by SEQ ID NO: 173377.

The probes having the nucleotide sequence represented by SEQ ID NOS: 177365, 177366, 177367, 177368, 177369, 177370, 177371, 177372, 177373, 177374, 177375 were selected from the ORFs having the nucleotide sequence represented by SEQ ID NO: 173378.

The probes having the nucleotide sequence represented by SEQ ID NOS: 177376, 177377, 177378, 177379, 177380, 177381, 177382, 177383, 177384, 177385, 177386 were selected from the ORFs having the nucleotide sequence represented by SEQ ID NO: 173379.

The probes having the nucleotide sequence represented by SEQ ID NOS: 177387, 177388, 177389, 177390, 177391, 177392, 177393, 177394, 177395, 177396, 177397 were selected from the ORFs having the nucleotide sequence represented by SEQ ID NO: 173380.

The probes having the nucleotide sequence represented by SEQ ID NOS: 177398, 177399, 177400, 177401, 177402, 177403, 177404, 177405, 177406, 177407, 177408 were selected from the ORFs having the nucleotide sequence represented by SEQ ID NO: 173381.

The probes having the nucleotide sequence represented by SEQ ID NOS: 177409, 177410, 177411, 177412, 177413, 177414, 177415, 177416, 177417, 177418, 177419 were selected from the ORFs having the nucleotide sequence represented by SEQ ID NO: 173382.

The probes having the nucleotide sequence represented by SEQ ID NOS: 177420, 177421, 177422, 177423, 177424, 177425, 177426, 177427, 177428, 177429, 177430 were selected from the ORFs having the nucleotide sequence represented by SEQ ID NO: 173383.

The probes having the nucleotide sequence represented by SEQ ID NOS: 177431, 177432, 177433, 177434, 177435, 177436, 177437, 177438, 177439, 177440, 177441 were selected from the ORFs having the nucleotide sequence represented by SEQ ID NO: 173384.

The probes having the nucleotide sequence represented by SEQ ID NOS: 177442, 177443, 177444, 177445, 177446, 177447, 177448, 177449, 177450, 177451, 177452 were selected from the ORFs having the nucleotide sequence represented by SEQ ID NO: 173385.

The probes having the nucleotide sequence represented by SEQ ID NOS: 177453, 177454, 177455, 177456, 177457, 177458, 177459, 177460, 177461, 177462, 177463 were selected from the ORFs having the nucleotide sequence represented by SEQ ID NO: 173386.

The probes having the nucleotide sequence represented by SEQ ID NOS: 177464, 177465, 177466, 177467, 177468, 177469, 177470, 177471, 177472, 177473, 177474 were selected from the ORFs having the nucleotide sequence represented by SEQ ID NO: 173387.

The probes having the nucleotide sequence represented by SEQ ID NOS: 177475, 177476, 177477, 177478, 177479, 177480, 177481, 177482, 177483, 177484, 177485 were selected from the ORFs having the nucleotide sequence represented by SEQ ID NO: 173388.

The probes having the nucleotide sequence represented by SEQ ID NOS: 177486, 177487, 177488, 177489, 177490, 177491, 177492, 177493, 177494, 177495, 177496 were selected from the ORFs having the nucleotide sequence represented by SEQ ID NO: 173389.

The probes having the nucleotide sequence represented by SEQ ID NOS: 177497, 177498, 177499, 177500, 177501, 177502, 177503, 177504, 177505, 177506, 177507 were selected from the ORFs having the nucleotide sequence represented by SEQ ID NO: 173390.

The probes having the nucleotide sequence represented by SEQ ID NOS: 177508, 177509, 177510, 177511, 177512, 177513, 177514, 177515, 177516, 177517, 177518 were selected from the ORFs having the nucleotide sequence represented by SEQ ID NO: 173391.

The probes having the nucleotide sequence represented by SEQ ID NOS: 177519, 177520, 177521, 177522, 177523, 177524, 177525, 177526, 177527, 177528, 177529 were selected from the ORFs having the nucleotide sequence represented by SEQ ID NO: 173392.

The probes having the nucleotide sequence represented by SEQ ID NOS: 177530, 177531, 177532, 177533, 177534, 177535, 177536, 177537, 177538, 177539, 177540 were selected from the ORFs having the nucleotide sequence represented by SEQ ID NO: 173393.

The probes having the nucleotide sequence represented by SEQ ID NOS: 177541, 177542, 177543, 177544, 177545, 177546, 177547, 177548, 177549, 177550, 177551 were selected from the ORFs having the nucleotide sequence represented by SEQ ID NO: 173394.

The probes having the nucleotide sequence represented by SEQ ID NOS: 177552, 177553, 177554, 177555, 177556, 177557, 177558, 177559, 177560, 177561, 177562 were selected from the ORFs having the nucleotide sequence represented by SEQ ID NO: 173395.

The probes having the nucleotide sequence represented by SEQ ID NOS: 177563, 177564, 177565, 177566, 177567, 177568, 177569, 177570, 177571, 177572, 177573 were selected from the ORFs having the nucleotide sequence represented by SEQ ID NO: 173396.

The probes having the nucleotide sequence represented by SEQ ID NOS: 177574, 177575, 177576, 177577, 177578, 177579, 177580, 177581, 177582, 177583, 177584 were selected from the ORFs having the nucleotide sequence represented by SEQ ID NO: 173397.

The probes having the nucleotide sequence represented by SEQ ID NOS: 177585, 177586, 177587, 177588, 177589, 177590, 177591, 177592, 177593, 177594, 177595 were selected from the ORFs having the nucleotide sequence represented by SEQ ID NO: 173398.

The probes having the nucleotide sequence represented by SEQ ID NOS: 177596, 177597, 177598, 177599, 177600, 177601, 177602, 177603, 177604, 177605, 177606 were selected from the ORFs having the nucleotide sequence represented by SEQ ID NO: 173399.

The probes having the nucleotide sequence represented by SEQ ID NOS: 177607, 177608, 177609, 177610, 177611, 177612, 177613, 177614, 177615, 177616, 177617 were selected from the ORFs having the nucleotide sequence represented by SEQ ID NO: 173400.

The probes having the nucleotide sequence represented by SEQ ID NOS: 177618, 177619, 177620, 177621, 177622, 177623, 177624, 177625, 177626, 177627, 177628 were selected from the ORFs having the nucleotide sequence represented by SEQ ID NO: 173401.

The probes having the nucleotide sequence represented by SEQ ID NOS: 177629, 177630, 177631, 177632, 177633, 177634, 177635, 177636, 177637, 177638, 177639 were selected from the ORFs having the nucleotide sequence represented by SEQ ID NO: 173402.

The probes having the nucleotide sequence represented by SEQ ID NOS: 177640, 177641, 177642, 177643, 177644, 177645, 177646, 177647, 177648, 177649, 177650 were selected from the ORFs having the nucleotide sequence represented by SEQ ID NO: 173403.

The probes having the nucleotide sequence represented by SEQ ID NOS: 177651, 177652, 177653, 177654, 177655, 177656, 177657, 177658, 177659, 177660, 177661 were selected from the ORFs having the nucleotide sequence represented by SEQ ID NO: 173404.

The probes having the nucleotide sequence represented by SEQ ID NOS: 177662, 177663, 177664, 177665, 177666, 177667, 177668, 177669, 177670, 177671, 177672 were selected from the ORFs having the nucleotide sequence represented by SEQ ID NO: 173405.

The probes having the nucleotide sequence represented by SEQ ID NOS: 177673, 177674, 177675, 177676, 177677, 177678, 177679, 177680, 177681, 177682, 177683 were selected from the ORFs having the nucleotide sequence represented by SEQ ID NO: 173406.

The probes having the nucleotide sequence represented by SEQ ID NOS: 177684, 177685, 177686, 177687, 177688, 177689, 177690, 177691, 177692, 177693, 177694 were selected from the ORFs having the nucleotide sequence represented by SEQ ID NO: 173407.

The probes having the nucleotide sequence represented by SEQ ID NOS: 177695, 177696, 177697, 177698, 177699, 177700, 177701, 177702, 177703, 177704, 177705 were selected from the ORFs having the nucleotide sequence represented by SEQ ID NO: 173408.

The probes having the nucleotide sequence represented by SEQ ID NOS: 177706, 177707, 177708, 177709, 177710, 177711, 177712, 177713, 177714, 177715, 177716 were selected from the ORFs having the nucleotide sequence represented by SEQ ID NO: 173409.

The probes having the nucleotide sequence represented by SEQ ID NOS: 177717, 177718, 177719, 177720, 177721, 177722, 177723, 177724, 177725 were selected from the ORFs having the nucleotide sequence represented by SEQ ID NO: 173410.

The probes having the nucleotide sequence represented by SEQ ID NOS: 177726, 177727, 177728, 177729, 177730, 177731, 177732, 177733, 177734, 177735, 177736 were selected from the ORFs having the nucleotide sequence represented by SEQ ID NO: 173411.

The probes having the nucleotide sequence represented by SEQ ID NOS: 177737, 177738, 177739, 177740, 177741, 177742, 177743, 177744, 177745 were selected from the ORFs having the nucleotide sequence represented by SEQ ID NO: 173412.

The probes having the nucleotide sequence represented by SEQ ID NOS: 177746, 177747, 177748, 177749, 177750, 177751, 177752, 177753, 177754, 177755, 177756 were selected from the ORFs having the nucleotide sequence represented by SEQ ID NO: 173413.

The probes having the nucleotide sequence represented by SEQ ID NOS: 177757, 177758, 177759, 177760, 177761, 177762, 177763, 177764, 177765, 177766, 177767 were selected from the ORFs having the nucleotide sequence represented by SEQ ID NO: 173414.

The probes having the nucleotide sequence represented by SEQ ID NOS: 177768, 177769, 177770, 177771, 177772, 177773, 177774, 177775, 177776, 177777, 177778 were selected from the ORFs having the nucleotide sequence represented by SEQ ID NO: 173415.

The probes having the nucleotide sequence represented by SEQ ID NOS: 177779, 177780, 177781, 177782, 177783, 177784, 177785, 177786, 177787, 177788, 177789 were selected from the ORFs having the nucleotide sequence represented by SEQ ID NO: 173416.

The probes having the nucleotide sequence represented by SEQ ID NOS: 177790, 177791, 177792, 177793, 177794, 177795, 177796, 177797, 177798, 177799, 177800 were selected from the ORFs having the nucleotide sequence represented by SEQ ID NO: 173417.

The probes having the nucleotide sequence represented by SEQ ID NOS: 177801, 177802, 177803, 177804, 177805, 177806, 177807, 177808, 177809, 177810, 177811 were selected from the ORFs having the nucleotide sequence represented by SEQ ID NO: 173418.

The probes having the nucleotide sequence represented by SEQ ID NOS: 177812, 177813, 177814, 177815, 177816, 177817, 177818, 177819, 177820, 177821, 177822 were selected from the ORFs having the nucleotide sequence represented by SEQ ID NO: 173419.

The probes having the nucleotide sequence represented by SEQ ID NOS: 177823, 177824, 177825, 177826, 177827, 177828, 177829, 177830, 177831, 177832, 177833 were selected from the ORFs having the nucleotide sequence represented by SEQ ID NO: 173420.

The probes having the nucleotide sequence represented by SEQ ID NOS: 177834, 177835, 177836, 177837, 177838, 177839, 177840, 177841, 177842, 177843, 177844 were selected from the ORFs having the nucleotide sequence represented by SEQ ID NO: 173421.

The probes having the nucleotide sequence represented by SEQ ID NOS: 177845, 177846, 177847, 177848, 177849, 177850, 177851, 177852, 177853, 177854, 177855 were selected from the ORFs having the nucleotide sequence represented by SEQ ID NO: 173422.

The probes having the nucleotide sequence represented by SEQ ID NOS: 177856, 177857, 177858, 177859, 177860, 177861, 177862, 177863, 177864, 177865, 177866 were selected from the ORFs having the nucleotide sequence represented by SEQ ID NO: 173423.

The probes having the nucleotide sequence represented by SEQ ID NOS: 177867, 177868, 177869, 177870, 177871, 177872, 177873, 177874, 177875, 177876, 177877 were selected from the ORFs having the nucleotide sequence represented by SEQ ID NO: 173424.

The probes having the nucleotide sequence represented by SEQ ID NOS: 177878, 177879, 177880, 177881, 177882, 177883, 177884, 177885, 177886, 177887, 177888 were selected from the ORFs having the nucleotide sequence represented by SEQ ID NO: 173425.

The probes having the nucleotide sequence represented by SEQ ID NOS: 177889, 177890, 177891, 177892, 177893, 177894, 177895, 177896, 177897, 177898, 177899 were selected from the ORFs having the nucleotide sequence represented by SEQ ID NO: 173426.

The probes having the nucleotide sequence represented by SEQ ID NOS: 177900, 177901, 177902, 177903, 177904, 177905, 177906, 177907, 177908, 177909, 177910 were selected from the ORFs having the nucleotide sequence represented by SEQ ID NO: 173427.

The probes having the nucleotide sequence represented by SEQ ID NOS: 177911, 177912, 177913, 177914, 177915, 177916, 177917, 177918, 177919, 177920, 177921 were selected from the ORFs having the nucleotide sequence represented by SEQ ID NO: 173428.

The probes having the nucleotide sequence represented by SEQ ID NOS: 177922, 177923, 177924, 177925, 177926, 177927, 177928, 177929, 177930, 177931, 177932 were selected from the ORFs having the nucleotide sequence represented by SEQ ID NO: 173429.

The probes having the nucleotide sequence represented by SEQ ID NOS: 177933, 177934, 177935, 177936, 177937, 177938, 177939, 177940, 177941, 177942, 177943 were selected from the ORFs having the nucleotide sequence represented by SEQ ID NO: 173430.

The probes having the nucleotide sequence represented by SEQ ID NOS: 177944, 177945, 177946, 177947, 177948, 177949, 177950, 177951, 177952, 177953, 177954 were selected from the ORFs having the nucleotide sequence represented by SEQ ID NO: 173431.

The probes having the nucleotide sequence represented by SEQ ID NOS: 177955, 177956, 177957, 177958, 177959, 177960, 177961, 177962, 177963, 177964, 177965 were selected from the ORFs having the nucleotide sequence represented by SEQ ID NO: 173432.

The probes having the nucleotide sequence represented by SEQ ID NOS: 177966, 177967, 177968, 177969, 177970, 177971, 177972, 177973, 177974, 177975, 177976 were selected from the ORFs having the nucleotide sequence represented by SEQ ID NO: 173433.

The probes having the nucleotide sequence represented by SEQ ID NOS: 177977, 177978, 177979, 177980, 177981, 177982, 177983, 177984 were selected from the ORFs having the nucleotide sequence represented by SEQ ID NO: 173434.

The probes having the nucleotide sequence represented by SEQ ID NOS: 177985, 177986, 177987; 177988, 177989, 177990, 177991, 177992, 177993, 177994, 177995 were selected from the ORFs having the nucleotide sequence represented by SEQ ID NO: 173435.

The probes having the nucleotide sequence represented by SEQ ID NOS: 177996, 177997, 177998, 177999, 178000, 178001, 178002, 178003, 178004, 178005, 178006 were selected from the ORFs having the nucleotide sequence represented by SEQ ID NO: 173436.

The probes having the nucleotide sequence represented by SEQ ID NOS: 178007, 178008, 178009, 178010, 178011, 178012, 178013, 178014, 178015, 178016, 178017 were selected from the ORFs having the nucleotide sequence represented by SEQ ID NO: 173437.

The probes having the nucleotide sequence represented by SEQ ID NOS: 178018, 178019, 178020, 178021, 178022, 178023, 178024, 178025, 178026, 178027, 178028 were selected from the ORFs having the nucleotide sequence represented by SEQ ID NO: 173438.

The probes having the nucleotide sequence represented by SEQ ID NOS: 178029, 178030, 178031, 178032, 178033, 178034, 178035, 178036, 178037, 178038, 178039 were selected from the ORFs having the nucleotide sequence represented by SEQ ID NO: 173439.

The probes having the nucleotide sequence represented by SEQ ID NOS: 178040, 178041, 178042, 178043, 178044, 178045, 178046, 178047, 178048, 178049, 178050 were selected from the ORFs having the nucleotide sequence represented by SEQ ID NO: 173440.

The probes having the nucleotide sequence represented by SEQ ID NOS: 178051, 178052, 178053, 178054, 178055, 178056, 178057, 178058, 178059, 178060, 178061 were selected from the ORFs having the nucleotide sequence represented by SEQ ID NO: 173441.

The probes having the nucleotide sequence represented by SEQ ID NOS: 178062, 178063, 178064, 178065, 178066, 178067, 178068, 178069, 178070, 178071, 178072 were selected from the ORFs having the nucleotide sequence represented by SEQ ID NO: 173442.

The probes having the nucleotide sequence represented by SEQ ID NOS: 178073, 178074, 178075, 178076, 178077, 178078, 178079, 178080, 178081, 178082, 178083 were selected from the ORFs having the nucleotide sequence represented by SEQ ID NO: 173443.

The probes having the nucleotide sequence represented by SEQ ID NOS: 178084, 178085, 178086, 178087, 178088, 178089, 178090, 178091, 178092, 178093, 178094 were selected from the ORFs having the nucleotide sequence represented by SEQ ID NO: 173444.

The probes having the nucleotide sequence represented by SEQ ID NOS: 178095, 178096, 178097, 178098, 178099, 178100, 178101, 178102, 178103, 178104, 178105 were selected from the ORFs having the nucleotide sequence represented by SEQ ID NO: 173445.

The probes having the nucleotide sequence represented by SEQ ID NOS: 178106, 178107, 178108, 178109, 178110, 178111, 178112, 178113, 178114, 178115, 178116 were selected from the ORFs having the nucleotide sequence represented by SEQ ID NO: 173446.

The probes having the nucleotide sequence represented by SEQ ID NOS: 178117, 178118, 178119, 178120, 178121, 178122, 178123, 178124, 178125, 178126, 178127 were selected from the ORFs having the nucleotide sequence represented by SEQ ID NO: 173447.

The probes having the nucleotide sequence represented by SEQ ID NOS: 178128, 178129, 178130, 178131, 178132, 178133, 178134, 178135, 178136, 178137, 178138 were selected from the ORFs having the nucleotide sequence represented by SEQ ID NO: 173448.

The probes having the nucleotide sequence represented by SEQ ID NOS: 178139, 178140, 178141, 178142, 178143, 178144, 178145, 178146, 178147, 178148, 178149 were selected from the ORFs having the nucleotide sequence represented by SEQ ID NO: 173449.

The probes having the nucleotide sequence represented by SEQ ID NOS: 178150, 178151, 178152, 178153, 178154, 178155, 178156, 178157, 178158, 178159, 178160 were selected from the ORFs having the nucleotide sequence represented by SEQ ID NO: 173450.

The probes having the nucleotide sequence represented by SEQ ID NOS: 178161, 178162, 178163, 178164, 178165, 178166, 178167, 178168, 178169, 178170, 178171 were selected from the ORFs having the nucleotide sequence represented by SEQ ID NO: 173451.

The probes having the nucleotide sequence represented by SEQ ID NOS: 178172, 178173, 178174, 178175, 178176, 178177, 178178, 178179, 178180, 178181, 178182 were selected from the ORFs having the nucleotide sequence represented by SEQ ID NO: 173452.

The probes having the nucleotide sequence represented by SEQ ID NOS: 178183, 178184, 178185, 178186, 178187, 178188, 178189, 178190, 178191, 178192, 178193 were selected from the ORFs having the nucleotide sequence represented by SEQ ID NO: 173453.

The probes having the nucleotide sequence represented by SEQ ID NOS: 178194, 178195, 178196, 178197, 178198, 178199, 178200, 178201, 178202, 178203, 178204 were selected from the ORFs having the nucleotide sequence represented by SEQ ID NO: 173454.

The probes having the nucleotide sequence represented by SEQ ID NOS: 178205, 178206, 178207, 178208, 178209, 178210, 178211, 178212, 178213, 178214, 178215 were selected from the ORFs having the nucleotide sequence represented by SEQ ID NO: 173455.

The probes having the nucleotide sequence represented by SEQ ID NOS: 178216, 178217, 178218, 178219, 178220, 178221, 178222, 178223, 178224, 178225, 178226 were selected from the ORFs having the nucleotide sequence represented by SEQ ID NO: 173456.

The probes having the nucleotide sequence represented by SEQ ID NOS: 178227, 178228, 178229, 178230, 178231, 178232, 178233, 178234, 178235, 178236, 178237 were selected from the ORFs having the nucleotide sequence represented by SEQ ID NO: 173457.

The probes having the nucleotide sequence represented by SEQ ID NOS: 178238, 178239, 178240, 178241, 178242, 178243, 178244, 178245, 178246, 178247, 178248 were selected from the ORFs having the nucleotide sequence represented by SEQ ID NO: 173458.

The probes having the nucleotide sequence represented by SEQ ID NOS: 178249, 178250, 178251, 178252, 178253, 178254, 178255, 178256, 178257, 178258, 178259 were selected from the ORFs having the nucleotide sequence represented by SEQ ID NO: 173459.

The probes having the nucleotide sequence represented by SEQ ID NOS: 178260, 178261, 178262, 178263, 178264, 178265, 178266, 178267, 178268, 178269, 178270 were selected from the ORFs having the nucleotide sequence represented by SEQ ID NO: 173460.

The probes having the nucleotide sequence represented by SEQ ID NOS: 178271, 178272, 178273, 178274, 178275, 178276, 178277, 178278, 178279, 178280, 178281 were selected from the ORFs having the nucleotide sequence represented by SEQ ID NO: 173461.

The probes having the nucleotide sequence represented by SEQ ID NOS: 178282, 178283, 178284, 178285, 178286, 178287, 178288, 178289, 178290, 178291, 178292 were selected from the ORFs having the nucleotide sequence represented by SEQ ID NO: 173462.

The probes having the nucleotide sequence represented by SEQ ID NOS: 178293, 178294, 178295, 178296, 178297, 178298, 178299, 178300, 178301, 178302, 178303 were selected from the ORFs having the nucleotide sequence represented by SEQ ID NO: 173463.

The probes having the nucleotide sequence represented by SEQ ID NOS: 178304, 178305, 178306, 178307, 178308, 178309, 178310, 178311, 178312, 178313, 178314 were selected from the ORFs having the nucleotide sequence represented by SEQ ID NO: 173464.

The probes having the nucleotide sequence represented by SEQ ID NOS: 178315, 178316, 178317, 178318, 178319, 178320, 178321, 178322, 178323, 178324, 178325 were selected from the ORFs having the nucleotide sequence represented by SEQ ID NO: 173465.

The probes having the nucleotide sequence represented by SEQ ID NOS: 178326, 178327, 178328, 178329, 178330, 178331, 178332, 178333, 178334, 178335, 178336 were selected from the ORFs having the nucleotide sequence represented by SEQ ID NO: 173466.

The probes having the nucleotide sequence represented by SEQ ID NOS: 178337, 178338, 178339, 178340, 178341, 178342, 178343, 178344, 178345, 178346, 178347 were selected from the ORFs having the nucleotide sequence represented by SEQ ID NO: 173467.

The probes having the nucleotide sequence represented by SEQ ID NOS: 178348, 178349, 178350, 178351, 178352, 178353, 178354, 178355, 178356, 178357, 178358 were selected from the ORFs having the nucleotide sequence represented by SEQ ID NO: 173468.

The probes having the nucleotide sequence represented by SEQ ID NOS: 178359, 178360, 178361, 178362, 178363, 178364, 178365, 178366, 178367, 178368, 178369 were selected from the ORFs having the nucleotide sequence represented by SEQ ID NO: 173469.

The probes having the nucleotide sequence represented by SEQ ID NOS: 178370, 178371, 178372, 178373, 178374, 178375, 178376, 178377, 178378, 178379, 178380 were selected from the ORFs having the nucleotide sequence represented by SEQ ID NO: 173470.

The probes having the nucleotide sequence represented by SEQ ID NOS: 178381, 178382, 178383, 178384, 178385, 178386, 178387, 178388, 178389, 178390, 178391 were selected from the ORFs having the nucleotide sequence represented by SEQ ID NO: 173471.

The probes having the nucleotide sequence represented by SEQ ID NOS: 178392, 178393, 178394, 178395, 178396, 178397, 178398, 178399, 178400, 178401, 178402 were selected from the ORFs having the nucleotide sequence represented by SEQ ID NO: 173472.

The probes having the nucleotide sequence represented by SEQ ID NOS: 178403, 178404, 178405, 178406, 178407, 178408, 178409, 178410, 178411, 178412, 178413 were selected from the ORFs having the nucleotide sequence represented by SEQ ID NO: 173473.

The probes having the nucleotide sequence represented by SEQ ID NOS: 178414, 178415, 178416, 178417, 178418, 178419, 178420, 178421, 178422, 178423, 178424 were selected from the ORFs having the nucleotide sequence represented by SEQ ID NO: 173474.

The probes having the nucleotide sequence represented by SEQ ID NOS: 178425, 178426, 178427, 178428, 178429, 178430, 178431, 178432, 178433, 178434, 178435 were selected from the ORFs having the nucleotide sequence represented by SEQ ID NO: 173475.

The probes having the nucleotide sequence represented by SEQ ID NOS: 178436, 178437, 178438, 178439, 178440, 178441, 178442, 178443, 178444, 178445, 178446 were selected from the ORFs having the nucleotide sequence represented by SEQ ID NO: 173476.

The probes having the nucleotide sequence represented by SEQ ID NOS: 178447, 178448, 178449, 178450, 178451, 178452, 178453, 178454, 178455, 178456, 178457 were selected from the ORFs having the nucleotide sequence represented by SEQ ID NO: 173477.

The probes having the nucleotide sequence represented by SEQ ID NOS: 178458, 178459, 178460, 178461, 178462, 178463, 178464, 178465, 178466, 178467, 178468 were selected from the ORFs having the nucleotide sequence represented by SEQ ID NO: 173478.

The probes having the nucleotide sequence represented by SEQ ID NOS: 178469, 178470, 178471, 178472, 178473, 178474, 178475, 178476, 178477, 178478, 178479 were selected from the ORFs having the nucleotide sequence represented by SEQ ID NO: 173479.

The probes having the nucleotide sequence represented by SEQ ID NOS: 178480, 178481, 178482, 178483, 178484, 178485, 178486, 178487, 178488, 178489, 178490 were selected from the ORFs having the nucleotide sequence represented by SEQ ID NO: 173480.

The probes having the nucleotide sequence represented by SEQ ID NOS: 178491, 178492, 178493, 178494, 178495, 178496, 178497, 178498, 178499, 178500, 178501 were selected from the ORFs having the nucleotide sequence represented by SEQ ID NO: 173481.

The probes having the nucleotide sequence represented by SEQ ID NOS: 178502, 178503, 178504, 178505, 178506, 178507, 178508, 178509, 178510, 178511, 178512 were selected from the ORFs having the nucleotide sequence represented by SEQ ID NO: 173482.

The probes having the nucleotide sequence represented by SEQ ID NOS: 178513, 178514, 178515, 178516, 178517, 178518, 178519, 178520, 178521, 178522, 178523 were selected from the ORFs having the nucleotide sequence represented by SEQ ID NO: 173483.

The probes having the nucleotide sequence represented by SEQ ID NOS: 178524, 178525, 178526, 178527, 178528, 178529, 178530, 178531, 178532, 178533, 178534 were selected from the ORFs having the nucleotide sequence represented by SEQ ID NO: 173484.

The probes having the nucleotide sequence represented by SEQ ID NOS: 178535, 178536, 178537, 178538, 178539, 178540, 178541, 178542, 178543, 178544, 178545 were selected from the ORFs having the nucleotide sequence represented by SEQ ID NO: 173485.

The probes having the nucleotide sequence represented by SEQ ID NOS: 178546, 178547, 178548, 178549, 178550, 178551, 178552, 178553, 178554, 178555, 178556 were selected from the ORFs having the nucleotide sequence represented by SEQ ID NO: 173486.

The probes having the nucleotide sequence represented by SEQ ID NOS: 178557, 178558, 178559, 178560, 178561, 178562, 178563, 178564, 178565, 178566, 178567 were selected from the ORFs having the nucleotide sequence represented by SEQ ID NO: 173487.

The probes having the nucleotide sequence represented by SEQ ID NOS: 178568, 178569, 178570, 178571, 178572, 178573, 178574, 178575, 178576, 178577, 178578 were selected from the ORFs having the nucleotide sequence represented by SEQ ID NO: 173488.

The probes having the nucleotide sequence represented by SEQ ID NOS: 178579, 178580, 178581, 178582, 178583, 178584, 178585, 178586, 178587, 178588, 178589 were selected from the ORFs having the nucleotide sequence represented by SEQ ID NO: 173489.

The probes having the nucleotide sequence represented by SEQ ID NOS: 178590, 178591, 178592, 178593, 178594, 178595, 178596, 178597, 178598, 178599, 178600 were selected from the ORFs having the nucleotide sequence represented by SEQ ID NO: 173490.

The probes having the nucleotide sequence represented by SEQ ID NOS: 178601, 178602, 178603, 178604, 178605, 178606, 178607, 178608, 178609, 178610, 178611 were selected from the ORFs having the nucleotide sequence represented by SEQ ID NO: 173491.

The probes having the nucleotide sequence represented by SEQ ID NOS: 178612, 178613, 178614, 178615, 178616, 178617, 178618, 178619, 178620, 178621, 178622 were selected from the ORFs having the nucleotide sequence represented by SEQ ID NO: 173492.

The probes having the nucleotide sequence represented by SEQ ID NOS: 178623, 178624, 178625, 178626, 178627, 178628, 178629, 178630, 178631, 178632, 178633 were selected from the ORFs having the nucleotide sequence represented by SEQ ID NO: 173493.

The probes having the nucleotide sequence represented by SEQ ID NOS: 178634, 178635, 178636, 178637, 178638, 178639, 178640, 178641, 178642, 178643, 178644 were selected from the ORFs having the nucleotide sequence represented by SEQ ID NO: 173494.

The probes having the nucleotide sequence represented by SEQ ID NOS: 178645, 178646, 178647, 178648, 178649, 178650, 178651, 178652, 178653, 178654, 178655 were selected from the ORFs having the nucleotide sequence represented by SEQ ID NO: 173495.

The probes having the nucleotide sequence represented by SEQ ID NOS: 178656, 178657, 178658, 178659, 178660, 178661, 178662, 178663, 178664, 178665, 178666 were selected from the ORFs having the nucleotide sequence represented by SEQ ID NO: 173496.

The probes having the nucleotide sequence represented by SEQ ID NOS: 178667, 178668, 178669, 178670, 178671, 178672, 178673, 178674, 178675, 178676, 178677 were selected from the ORFs having the nucleotide sequence represented by SEQ ID NO: 173497.

The probes having the nucleotide sequence represented by SEQ ID NOS: 178678, 178679, 178680, 178681, 178682, 178683, 178684, 178685, 178686, 178687, 178688 were selected from the ORFs having the nucleotide sequence represented by SEQ ID NO: 173498.

The probes having the nucleotide sequence represented by SEQ ID NOS: 178689, 178690, 178691, 178692, 178693, 178694, 178695, 178696, 178697, 178698, 178699 were selected from the ORFs having the nucleotide sequence represented by SEQ ID NO: 173499.

The probes having the nucleotide sequence represented by SEQ ID NOS: 178700, 178701, 178702, 178703, 178704, 178705, 178706, 178707, 178708, 178709, 178710 were selected from the ORFs having the nucleotide sequence represented by SEQ ID NO: 173500.

The probes having the nucleotide sequence represented by SEQ ID NOS: 178711, 178712, 178713, 178714, 178715, 178716, 178717, 178718, 178719, 178720, 178721 were selected from the ORFs having the nucleotide sequence represented by SEQ ID NO: 173501.

The probes having the nucleotide sequence represented by SEQ ID NOS: 178722, 178723, 178724, 178725, 178726, 178727, 178728, 178729, 178730, 178731, 178732 were selected from the ORFs having the nucleotide sequence represented by SEQ ID NO: 173502.

The probes having the nucleotide sequence represented by SEQ ID NOS: 178733, 178734, 178735, 178736, 178737, 178738, 178739, 178740, 178741, 178742, 178743 were selected from the ORFs having the nucleotide sequence represented by SEQ ID NO: 173503.

The probes having the nucleotide sequence represented by SEQ ID NOS: 178744, 178745, 178746, 178747, 178748, 178749, 178750, 178751, 178752, 178753, 178754 were selected from the ORFs having the nucleotide sequence represented by SEQ ID NO: 173504.

The probes having the nucleotide sequence represented by SEQ ID NOS: 178755, 178756, 178757, 178758, 178759, 178760, 178761, 178762, 178763, 178764, 178765 were selected from the ORFs having the nucleotide sequence represented by SEQ ID NO: 173505.

The probes having the nucleotide sequence represented by SEQ ID NOS: 178766, 178767, 178768, 178769, 178770, 178771, 178772, 178773, 178774, 178775, 178776 were selected from the ORFs having the nucleotide sequence represented by SEQ ID NO: 173506.

The probes having the nucleotide sequence represented by SEQ ID NOS: 178777, 178778, 178779, 178780, 178781, 178782, 178783, 178784, 178785, 178786, 178787 were selected from the ORFs having the nucleotide sequence represented by SEQ ID NO: 173507.

The probes having the nucleotide sequence represented by SEQ ID NOS: 178788, 178789, 178790, 178791, 178792, 178793, 178794, 178795, 178796, 178797, 178798 were selected from the ORFs having the nucleotide sequence represented by SEQ ID NO: 173508.

The probes having the nucleotide sequence represented by SEQ ID NOS: 178799, 178800, 178801, 178802, 178803, 178804, 178805, 178806, 178807, 178808, 178809 were selected from the ORFs having the nucleotide sequence represented by SEQ ID NO: 173509.

The probes having the nucleotide sequence represented by SEQ ID NOS: 178810, 178811, 178812, 178813, 178814, 178815, 178816, 178817, 178818, 178819, 178820 were selected from the ORFs having the nucleotide sequence represented by SEQ ID NO: 173510.

The probes having the nucleotide sequence represented by SEQ ID NOS: 178821, 178822, 178823, 178824, 178825, 178826, 178827, 178828, 178829, 178830, 178831 were selected from the ORFs having the nucleotide sequence represented by SEQ ID NO: 173511.

The probes having the nucleotide sequence represented by SEQ ID NOS: 178832, 178833, 178834, 178835, 178836, 178837, 178838, 178839, 178840, 178841, 178842 were selected from the ORFs having the nucleotide sequence represented by SEQ ID NO: 173512.

The probes having the nucleotide sequence represented by SEQ ID NOS: 178843, 178844, 178845, 178846, 178847, 178848, 178849, 178850, 178851, 178852, 178853 were selected from the ORFs having the nucleotide sequence represented by SEQ ID NO: 173513.

The probes having the nucleotide sequence represented by SEQ ID NOS: 178854, 178855, 178856, 178857, 178858, 178859, 178860, 178861, 178862, 178863, 178864 were selected from the ORFs having the nucleotide sequence represented by SEQ ID NO: 173514.

The probes having the nucleotide sequence represented by SEQ ID NOS: 178865, 178866, 178867, 178868, 178869, 178870, 178871, 178872, 178873, 178874, 178875 were selected from the ORFs having the nucleotide sequence represented by SEQ ID NO: 173515.

The probes having the nucleotide sequence represented by SEQ ID NOS: 178876, 178877, 178878, 178879, 178880, 178881, 178882, 178883, 178884, 178885, 178886 were selected from the ORFs having the nucleotide sequence represented by SEQ ID NO: 173516.

The probes having the nucleotide sequence represented by SEQ ID NOS: 178887, 178888, 178889, 178890, 178891, 178892, 178893, 178894, 178895, 178896, 178897 were selected from the ORFs having the nucleotide sequence represented by SEQ ID NO: 173517.

The probes having the nucleotide sequence represented by SEQ ID NOS: 178898, 178899, 178900, 178901, 178902, 178903, 178904, 178905, 178906, 178907, 178908 were selected from the ORFs having the nucleotide sequence represented by SEQ ID NO: 173518.

The probes having the nucleotide sequence represented by SEQ ID NOS: 178909, 178910, 178911, 178912, 178913, 178914, 178915, 178916, 178917, 178918, 178919 were selected from the ORFs having the nucleotide sequence represented by SEQ ID NO: 173519.

The probes having the nucleotide sequence represented by SEQ ID NOS: 178920, 178921, 178922, 178923, 178924, 178925, 178926, 178927, 178928, 178929, 178930 were selected from the ORFs having the nucleotide sequence represented by SEQ ID NO: 173520.

The probes having the nucleotide sequence represented by SEQ ID NOS: 178931, 178932, 178933, 178934, 178935, 178936, 178937, 178938, 178939, 178940, 178941 were selected from the ORFs having the nucleotide sequence represented by SEQ ID NO: 173521.

The probes having the nucleotide sequence represented by SEQ ID NOS: 178942, 178943, 178944, 178945, 178946, 178947, 178948, 178949, 178950, 178951, 178952 were selected from the ORFs having the nucleotide sequence represented by SEQ ID NO: 173522.

The probes having the nucleotide sequence represented by SEQ ID NOS: 178953, 178954, 178955, 178956, 178957, 178958, 178959, 178960, 178961, 178962, 178963 were selected from the ORFs having the nucleotide sequence represented by SEQ ID NO: 173523.

The probes having the nucleotide sequence represented by SEQ ID NOS: 178964, 178965, 178966, 178967, 178968, 178969, 178970, 178971, 178972, 178973, 178974 were selected from the ORFs having the nucleotide sequence represented by SEQ ID NO: 173524.

The probes having the nucleotide sequence represented by SEQ ID NOS: 178975, 178976, 178977, 178978, 178979, 178980, 178981, 178982, 178983, 178984, 178985 were selected from the ORFs having the nucleotide sequence represented by SEQ ID NO: 173525.

The probes having the nucleotide sequence represented by SEQ ID NOS: 178986, 178987, 178988, 178989, 178990, 178991, 178992, 178993, 178994, 178995, 178996 were selected from the ORFs having the nucleotide sequence represented by SEQ ID NO: 173526.

The probes having the nucleotide sequence represented by SEQ ID NOS: 178997, 178998, 178999, 179000, 179001, 179002, 179003, 179004, 179005, 179006, 179007 were selected from the ORFs having the nucleotide sequence represented by SEQ ID NO: 173527.

The probes having the nucleotide sequence represented by SEQ ID NOS: 179008, 179009, 179010, 179011, 179012, 179013, 179014, 179015, 179016, 179017, 179018 were selected from the ORFs having the nucleotide sequence represented by SEQ ID NO: 173528.

The probes having the nucleotide sequence represented by SEQ ID NOS: 179019, 179020, 179021, 179022, 179023, 179024, 179025, 179026, 179027, 179028, 179029 were selected from the ORFs having the nucleotide sequence represented by SEQ ID NO: 173529.

The probes having the nucleotide sequence represented by SEQ ID NOS: 179030, 179031, 179032, 179033, 179034, 179035, 179036, 179037, 179038, 179039, 179040 were selected from the ORFs having the nucleotide sequence represented by SEQ ID NO: 173530.

The probes having the nucleotide sequence represented by SEQ ID NOS: 179041, 179042, 179043, 179044, 179045, 179046, 179047, 179048, 179049, 179050, 179051 were selected from the ORFs having the nucleotide sequence represented by SEQ ID NO: 173531.

The probes having the nucleotide sequence represented by SEQ ID NOS: 179052, 179053, 179054, 179055, 179056, 179057, 179058, 179059, 179060, 179061, 179062 were selected from the ORFs having the nucleotide sequence represented by SEQ ID NO: 173532.

The probes having the nucleotide sequence represented by SEQ ID NOS: 179063, 179064, 179065, 179066, 179067, 179068, 179069, 179070, 179071, 179072, 179073 were selected from the ORFs having the nucleotide sequence represented by SEQ ID NO: 173533.

The probes having the nucleotide sequence represented by SEQ ID NOS: 179074, 179075, 179076, 179077, 179078, 179079, 179080, 179081, 179082, 179083, 179084 were selected from the ORFs having the nucleotide sequence represented by SEQ ID NO: 173534.

The probes having the nucleotide sequence represented by SEQ ID NOS: 179085, 179086, 179087, 179088, 179089, 179090, 179091, 179092, 179093, 179094, 179095 were selected from the ORFs having the nucleotide sequence represented by SEQ ID NO: 173535.

The probes having the nucleotide sequence represented by SEQ ID NOS: 179096, 179097, 179098, 179099, 179100, 179101, 179102, 179103, 179104, 179105, 179106 were selected from the ORFs having the nucleotide sequence represented by SEQ ID NO: 173536.

The probes having the nucleotide sequence represented by SEQ ID NOS: 179107, 179108, 179109, 179110, 179111, 179112, 179113, 179114, 179115, 179116, 179117 were selected from the ORFs having the nucleotide sequence represented by SEQ ID NO: 173537.

The probes having the nucleotide sequence represented by SEQ ID NOS: 179118, 179119, 179120, 179121, 179122, 179123, 179124, 179125, 179126, 179127, 179128 were selected from the ORFs having the nucleotide sequence represented by SEQ ID NO: 173538.

The probes having the nucleotide sequence represented by SEQ ID NOS: 179129, 179130, 179131, 179132, 179133, 179134, 179135, 179136, 179137, 179138, 179139 were selected from the ORFs having the nucleotide sequence represented by SEQ ID NO: 173539.

The probes having the nucleotide sequence represented by SEQ ID NOS: 179140, 179141, 179142, 179143, 179144, 179145, 179146, 179147, 179148, 179149, 179150 were selected from the ORFs having the nucleotide sequence represented by SEQ ID NO: 173540.

The probes having the nucleotide sequence represented by SEQ ID NOS: 179151, 179152, 179153, 179154, 179155, 179156, 179157, 179158, 179159, 179160, 179161 were selected from the ORFs having the nucleotide sequence represented by SEQ ID NO: 173541.

The probes having the nucleotide sequence represented by SEQ ID NOS: 179162, 179163, 179164, 179165, 179166, 179167, 179168, 179169, 179170, 179171, 179172 were selected from the ORFs having the nucleotide sequence represented by SEQ ID NO: 173542.

The probes having the nucleotide sequence represented by SEQ ID NOS: 179173, 179174, 179175, 179176, 179177, 179178, 179179, 179180, 179181, 179182, 179183 were selected from the ORFs having the nucleotide sequence represented by SEQ ID NO: 173543.

The probes having the nucleotide sequence represented by SEQ ID NOS: 179184, 179185, 179186, 179187, 179188, 179189, 179190, 179191, 179192, 179193, 179194 were selected from the ORFs having the nucleotide sequence represented by SEQ ID NO: 173544.

The probes having the nucleotide sequence represented by SEQ ID NOS: 179195, 179196, 179197, 179198, 179199, 179200, 179201, 179202, 179203, 179204, 179205 were selected from the ORFs having the nucleotide sequence represented by SEQ ID NO: 173545.

The probes having the nucleotide sequence represented by SEQ ID NOS: 179206, 179207, 179208, 179209, 179210, 179211, 179212, 179213, 179214, 179215, 179216 were selected from the ORFs having the nucleotide sequence represented by SEQ ID NO: 173546.

The probes having the nucleotide sequence represented by SEQ ID NOS: 179217, 179218, 179219, 179220, 179221, 179222, 179223, 179224, 179225, 179226, 179227 were selected from the ORFs having the nucleotide sequence represented by SEQ ID NO: 173547.

The probes having the nucleotide sequence represented by SEQ ID NOS: 179228, 179229, 179230, 179231, 179232, 179233, 179234, 179235, 179236, 179237, 179238 were selected from the ORFs having the nucleotide sequence represented by SEQ ID NO: 173548.

The probes having the nucleotide sequence represented by SEQ ID NOS: 179239, 179240, 179241, 179242, 179243, 179244, 179245, 179246, 179247, 179248, 179249 were selected from the ORFs having the nucleotide sequence represented by SEQ ID NO: 173549.

The probes having the nucleotide sequence represented by SEQ ID NOS: 179250, 179251, 179252, 179253, 179254, 179255, 179256, 179257, 179258, 179259, 179260 were selected from the ORFs having the nucleotide sequence represented by SEQ ID NO: 173550.

The probes having the nucleotide sequence represented by SEQ ID NOS: 179261, 179262, 179263, 179264, 179265, 179266, 179267, 179268, 179269, 179270, 179271 were selected from the ORFs having the nucleotide sequence represented by SEQ ID NO: 173551.

The probes having the nucleotide sequence represented by SEQ ID NOS: 179272, 179273, 179274, i79275, 179276, 179277, 179278, 179279, 179280, 179281, 179282 were selected from the ORFs having the nucleotide sequence represented by SEQ ID NO: 173552.

The probes having the nucleotide sequence represented by SEQ ID NOS: 179283, 179284, 179285, 179286, 179287, 179288, 179289, 179290, 179291, 179292, 179293 were selected from the ORFs having the nucleotide sequence represented by SEQ ID NO: 173553.

The probes having the nucleotide sequence represented by SEQ ID NOS: 179294, 179295, 179296, 179297, 179298, 179299, 179300, 179301, 179302, 179303, 179304 were selected from the ORFs having the nucleotide sequence represented by SEQ ID NO: 173554.

The probes having the nucleotide sequence represented by SEQ ID NOS: 179305, 179306, 179307, 179308, 179309, 179310, 179311, 179312, 179313, 179314, 179315 were selected from the ORFs having the nucleotide sequence represented by SEQ ID NO: 173555.

The probes having the nucleotide sequence represented by SEQ ID NOS: 179316, 179317, 179318, 179319, 179320, 179321, 179322, 179323, 179324, 179325, 179326 were selected from the ORFs having the nucleotide sequence represented by SEQ ID NO: 173556.

The probes having the nucleotide sequence represented by SEQ ID NOS: 179327, 179328, 179329, 179330, 179331, 179332, 179333, 179334, 179335, 179336, 179337 were selected from the ORFs having the nucleotide sequence represented by SEQ ID NO: 173557.

The probes having the nucleotide sequence represented by SEQ ID NOS: 179338, 179339, 179340, 179341, 179342, 179343, 179344, 179345, 179346, 179347, 179348 were selected from the ORFs having the nucleotide sequence represented by SEQ ID NO: 173558.

The probes having the nucleotide sequence represented by SEQ ID NOS: 179349, 179350, 179351, 179352, 179353, 179354, 179355, 179356, 179357, 179358, 179359 were selected from the ORFs having the nucleotide sequence represented by SEQ ID NO: 173559.

The probes having the nucleotide sequence represented by SEQ ID NOS: 179360, 179361, 179362, 179363, 179364, 179365, 179366, 179367, 179368, 179369, 179370 were selected from the ORFs having the nucleotide sequence represented by SEQ ID NO: 173560.

The probes having the nucleotide sequence represented by SEQ. ID NOS: 179371, 179372, 179373, 179374, 179375, 179376, 179377, 179378, 179379, 179380, 179381 were selected from the ORFs having the nucleotide sequence represented by SEQ ID NO: 173561.

The probes having the nucleotide sequence represented by SEQ ID NOS: 179382, 179383, 179384, 179385, 179386, 179387, 179388, 179389, 179390, 179391, 179392 were selected from the ORFs having the nucleotide sequence represented by SEQ ID NO: 173562.

The probes having the nucleotide sequence represented by SEQ ID NOS: 179393, 179394, 179395, 179396, 179397, 179398, 179399, 179400, 179401, 179402, 179403 were selected from the ORFs having the nucleotide sequence represented by SEQ ID NO: 173563.

The probes having the nucleotide sequence represented by SEQ ID NOS: 179404, 179405, 179406, 179407, 179408, 179409, 179410, 179411, 179412, 179413, 179414 were selected from the ORFs having the nucleotide sequence represented by SEQ ID NO: 173564.

The probes having the nucleotide sequence represented by SEQ ID NOS: 179415, 179416, 179417, 179418, 179419, 179420, 179421, 179422, 179423, 179424, 179425 were selected from the ORFs having the nucleotide sequence represented by SEQ ID NO: 173565.

The probes having the nucleotide sequence represented by SEQ ID NOS: 179426, 179427, 179428, 179429, 179430, 179431, 179432, 179433, 179434, 179435, 179436 were selected from the ORFs having the nucleotide sequence represented by SEQ ID NO: 173566.

The probes having the nucleotide sequence represented by SEQ ID NOS: 179437, 179438, 179439, 179440, 179441, 179442, 179443, 179444, 179445, 179446, 179447 were selected from the ORFs having the nucleotide sequence represented by SEQ ID NO: 173567.

The probes having the nucleotide sequence represented by SEQ ID NOS: 179448, 179449, 179450, 179451, 179452, 179453, 179454, 179455, 179456, 179457, 179458 were selected from the ORFs having the nucleotide sequence represented by SEQ ID NO: 173568.

The probes having the nucleotide sequence represented by SEQ ID NOS: 179459, 179460, 179461, 179462, 179463, 179464, 179465, 179466, 179467, 179468, 179469 were selected from the ORFs having the nucleotide sequence represented by SEQ ID NO: 173569.

The probes having the nucleotide sequence represented by SEQ ID NOS: 179470, 179471, 179472, 179473, 179474, 179475, 179476, 179477, 179478, 179479, 179480 were selected from the ORFs having the nucleotide sequence represented by SEQ ID NO: 173570.

The probes having the nucleotide sequence represented by SEQ ID NOS: 179481, 179482, 179483, 179484, 179485, 179486, 179487, 179488, 179489, 179490, 179491 were selected from the ORFs having the nucleotide sequence represented by SEQ ID NO: 173571.

The probes having the nucleotide sequence represented by SEQ ID NOS: 179492, 179493, 179494, 179495, 179496, 179497, 179498, 179499, 179500, 179501, 179502 were selected from the ORFs having the nucleotide sequence represented by SEQ ID NO: 173572.

The probes having the nucleotide sequence represented by SEQ ID NOS: 179503, 179504, 179505, 179506, 179507, 179508, 179509, 179510, 179511, 179512, 179513 were selected from the ORFs having the nucleotide sequence represented by SEQ ID NO: 173573.

The probes having the nucleotide sequence represented by SEQ ID NOS: 179514, 179515, 179516, 179517, 179518, 179519, 179520, 179521, 179522, 179523, 179524 were selected from the ORFs having the nucleotide sequence represented by SEQ ID NO: 173574.

The probes having the nucleotide sequence represented by SEQ ID NOS: 179525, 179526, 179527, 179528, 179529, 179530, 179531, 179532, 179533, 179534, 179535 were selected from the ORFs having the nucleotide sequence represented by SEQ ID NO: 173575.

The probes having the nucleotide sequence represented by SEQ ID NOS: 179536, 179537, 179538, 179539, 179540, 179541, 179542, 179543, 179544, 179545, 179546 were selected from the ORFs having the nucleotide sequence represented by SEQ ID NO: 173576.

The probes having the nucleotide sequence represented by SEQ ID NOS: 179547, 179548, 179549, 179550, 179551, 179552, 179553, 179554, 179555, 179556, 179557 were selected from the ORFs having the nucleotide sequence represented by SEQ ID NO: 173577.

The probes having the nucleotide sequence represented by SEQ ID NOS: 179558, 179559, 179560, 179561, 179562, 179563, 179564, 179565, 179566, 179567, 179568 were selected from the ORFs having the nucleotide sequence represented by SEQ ID NO: 173578.

The probes having the nucleotide sequence represented by SEQ ID NOS: 179569, 179570, 179571, 179572, 179573, 179574, 179575, 179576, 179577, 179578, 179579 were selected from the ORFs having the nucleotide sequence represented by SEQ ID NO: 173579.

The probes having the nucleotide sequence represented by SEQ ID NOS: 179580, 179581, 179582, 179583, 179584, 179585, 179586, 179587, 179588, 179589, 179590 were selected from the ORFs having the nucleotide sequence represented by SEQ ID NO: 173580.

The probes having the nucleotide sequence represented by SEQ ID NOS: 179591, 179592, 179593, 179594, 179595, 179596, 179597, 179598, 179599, 179600, 179601 were selected from the ORFs having the nucleotide sequence represented by SEQ ID NO: 173581.

The probes having the nucleotide sequence represented by SEQ ID NOS: 179602, 179603, 179604, 179605, 179606, 179607, 179608, 179609, 179610, 179611, 179612 were selected from the ORFs having the nucleotide sequence represented by SEQ ID NO: 173582.

The probes having the nucleotide sequence represented by SEQ ID NOS: 179613, 179614, 179615, 179616, 179617, 179618, 179619, 179620, 179621, 179622, 179623 were selected from the ORFs having the nucleotide sequence represented by SEQ ID NO: 173583.

The probes having the nucleotide sequence represented by SEQ ID NOS: 179624, 179625, 179626, 179627, 179628, 179629, 179630, 179631, 179632., 179633, 179634 were selected from the ORFs having the nucleotide sequence represented by SEQ ID NO: 173584.

The probes having the nucleotide sequence represented by SEQ ID NOS: 179635, 179636, 179637, 179638, 179639, 179640, 179641, 179642, 179643, 179644, 179645 were selected from the ORFs having the nucleotide sequence represented by SEQ ID NO: 173585.

The probes having the nucleotide sequence represented by SEQ ID NOS: 179646, 179647, 179648, 179649, 179650, 179651, 179652, 179653, 179654, 179655, 179656 were selected from the ORFs having the nucleotide sequence represented by SEQ ID NO: 173586.

The probes having the nucleotide sequence represented by SEQ ID NOS: 179657, 179658, 179659, 179660, 179661, 179662, 179663, 179664, 179665, 179666, 179667 were selected from the ORFs having the nucleotide sequence represented by SEQ ID NO: 173587.

The probes having the nucleotide sequence represented by SEQ ID NOS: 179668, 179669, 179670, 179671, 179672, 179673, 179674, 179675, 179676, 179677, 179678 were selected from the ORFs having the nucleotide sequence represented by SEQ ID NO: 173588.

The probes having the nucleotide sequence represented by SEQ ID NOS: 179679, 179680, 179681, 179682, 179683, 179684, 179685, 179686, 179687, 179688, 179689 were selected from the ORFs having the nucleotide sequence represented by SEQ ID NO: 173589.

The probes having the nucleotide sequence represented by SEQ ID NOS: 179690, 179691, 179692, 179693, 179694, 179695, 179696, 179697, 179698, 179699, 179700 were selected from the ORFs having the nucleotide sequence represented by SEQ ID NO: 173590.

The probes having the nucleotide sequence represented by SEQ ID NOS: 179701, 179702, 179703, 179704, 179705, 179706, 179707, 179708, 179709, 179710, 179711 were selected from the ORFs having the nucleotide sequence represented by SEQ ID NO: 173591.

The probes having the nucleotide sequence represented by SEQ ID NOS: 179712, 179713, 179714, 179715, 179716, 179717, 179718, 179719, 179720, 179721, 179722 were selected from the ORFs having the nucleotide sequence represented by SEQ ID NO: 173592.

The probes having the nucleotide sequence represented by SEQ ID NOS: 179723, 179724, 179725, 179726, 179727, 179728, 179729, 179730, 179731, 179732, 179733 were selected from the ORFs having the nucleotide sequence represented by SEQ ID NO: 173593.

The probes having the nucleotide sequence represented by SEQ ID NOS: 179734, 179735, 179736, 179737, 179738, 179739, 179740, 179741, 179742, 179743, 179744 were selected from the ORFs having the nucleotide sequence represented by SEQ ID NO: 173594.

The probes having the nucleotide sequence represented by SEQ ID NOS: 179745, 179746, 179747, 179748, 179749, 179750, 179751, 179752, 179753, 179754, 179755 were selected from the ORFs having the nucleotide sequence represented by SEQ ID NO: 173595.

The probes having the nucleotide sequence represented by SEQ ID NOS: 179756, 179757, 179758, 179759, 179760, 179761, 179762, 179763, 179764, 179765, 179766 were selected from the ORFs having the nucleotide sequence represented by SEQ ID NO: 173596.

The probes having the nucleotide sequence represented by SEQ ID NOS: 179767, 179768, 179769, 179770, 179771, 179772, 179773, 179774, 179775, 179776, 179777 were selected from the ORFs having the nucleotide sequence represented by SEQ ID NO: 173597.

The probes having the nucleotide sequence represented by SEQ ID NOS: 179778, 179779, 179780, 179781, 179782, 179783, 179784, 179785, 179786, 179787, 179788 were selected from the ORFs having the nucleotide sequence represented by SEQ ID NO: 173598.

The probes having the nucleotide sequence represented by SEQ ID NOS: 179789, 179790, 179791, 179792, 179793, 179794, 179795, 179796, 179797, 179798, 179799 were selected from the ORFs having the nucleotide sequence represented by SEQ ID NO: 173599.

The probes having the nucleotide sequence represented by SEQ ID NOS: 179800, 179801, 179802, 179803, 179804, 179805, 179806, 179807, 179808, 179809, 179810 were selected from the ORFs having the nucleotide sequence represented by SEQ ID NO: 173600.

The probes having the nucleotide sequence represented by SEQ ID NOS: 179811, 179812, 179813, 179814, 179815, 179816, 179817, 179818, 179819, 179820, 179821 were selected from the ORFs having the nucleotide sequence represented by SEQ ID NO: 173601.

The probes having the nucleotide sequence represented by SEQ ID NOS: 179822, 179823, 179824, 179825, 179826, 179827, 179828, 179829, 179830, 179831, 179832 were selected from the ORFs having the nucleotide sequence represented by SEQ ID NO: 173602.

The probes having the nucleotide sequence represented by SEQ ID NOS: 179833, 179834, 179835, 179836, 179837, 179838, 179839, 179840, 179841, 179842, 179843 were selected from the ORFs having the nucleotide sequence represented by SEQ ID NO: 173603.

The probes having the nucleotide sequence represented by SEQ ID NOS: 179844, 179845, 179846, 179847, 179848, 179849, 179850, 179851, 179852, 179853, 179854 were selected from the ORFs having the nucleotide sequence represented by SEQ ID NO: 173604.

The probes having the nucleotide sequence represented by SEQ ID NOS: 179855, 179856, 179857, 179858, 179859, 179860, 179861, 179862, 179863, 179864, 179865 were selected from the ORFs having the nucleotide sequence represented by SEQ ID NO: 173605.

The probes having the nucleotide sequence represented by SEQ ID NOS: 179866, 179867, 179868, 179869, 179870, 179871, 179872, 179873, 179874, 179875, 179876 were selected from the ORFs having the nucleotide sequence represented by SEQ ID NO: 173606.

The probes having the nucleotide sequence represented by SEQ ID NOS: 179877, 179878, 179879, 179880, 179881, 179882, 179883, 179884, 179885, 179886, 179887 were selected from the ORFs having the nucleotide sequence represented by SEQ ID NO: 173607.

The probes having the nucleotide sequence represented by SEQ ID NOS: 179888, 179889, 179890, 179891, 179892, 179893, 179894, 179895, 179896, 179897, 179898 were selected from the ORFs having the nucleotide sequence represented by SEQ ID NO: 173608.

The probes having the nucleotide sequence represented by SEQ ID NOS: 179899, 179900, 179901, 179902, 179903, 179904, 179905, 179906, 179907, 179908, 179909 were selected from the ORFs having the nucleotide sequence represented by SEQ ID NO: 173609.

The probes having the nucleotide sequence represented by SEQ ID NOS: 179910, 179911, 179912, 179913, 179914, 179915, 179916, 179917, 179918, 179919, 179920 were selected from the ORFs having the nucleotide sequence represented by SEQ ID NO: 173610.

The probes having the nucleotide sequence represented by SEQ ID NOS: 179921, 179922, 179923, 179924, 179925, 179926, 179927, 179928, 179929, 179930, 179931 were selected from the ORFs having the nucleotide sequence represented by SEQ ID NO: 173611.

The probes having the nucleotide sequence represented by SEQ ID NOS: 179932, 179933, 179934, 179935, 179936, 179937, 179938, 179939, 179940, 179941, 179942 were selected from the ORFs having the nucleotide sequence represented by SEQ ID NO: 173612.

The probes having the nucleotide sequence represented by SEQ ID NOS: 179943, 179944, 179945, 179946, 179947, 179948, 179949, 179950, 179951, 179952, 179953 were selected from the ORFs having the nucleotide sequence represented by SEQ ID NO: 173613.

The probes having the nucleotide sequence represented by SEQ ID NOS: 179954, 179955, 179956, 179957, 179958, 179959, 179960, 179961, 179962, 179963, 179964 were selected from the ORFs having the nucleotide sequence represented by SEQ ID NO: 173614.

The probes having the nucleotide sequence represented by SEQ ID NOS: 179965, 179966, 179967, 179968, 179969, 179970, 179971, 179972, 179973, 179974, 179975 were selected from the ORFs having the nucleotide sequence represented by SEQ ID NO: 173615.

The probes having the nucleotide sequence represented by SEQ ID NOS: 179976, 179977, 179978, 179979, 179980, 179981, 179982, 179983, 179984, 179985, 179986 were selected from the ORFs having the nucleotide sequence represented by SEQ ID NO: 173616.

The probes having the nucleotide sequence represented by SEQ ID NOS: 179987, 179988, 179989, 179990, 179991, 179992, 179993, 179994, 179995, 179996, 179997 were selected from the ORFs having the nucleotide sequence represented by SEQ ID NO: 173617.

The probes having the nucleotide sequence represented by SEQ ID NOS: 179998, 179999, 180000, 180001, 180002, 180003, 180004, 180005, 180006, 180007, 180008 were selected from the ORFs having the nucleotide sequence represented by SEQ ID NO: 173618.

The probes having the nucleotide sequence represented by SEQ ID NOS: 180009, 180010, 180011, 180012, 180013, 180014, 180015, 180016, 180017, 180018, 180019 were selected from the ORFs having the nucleotide sequence represented by SEQ ID NO: 173619.

The probes having the nucleotide sequence represented by SEQ ID NOS: 180020, 180021, 180022, 180023, 180024, 180025, 180026, 180027, 180028, 180029, 180030 were selected from the ORFs having the nucleotide sequence represented by SEQ ID NO: 173620.

The probes having the nucleotide sequence represented by SEQ ID NOS: 180031, 180032, 180033, 180034, 180035, 180036, 180037, 180038, 180039, 180040, 180041 were selected from the ORFs having the nucleotide sequence represented by SEQ ID NO: 173621.

The probes having the nucleotide sequence represented by SEQ ID NOS: 180042, 180043, 180044, 180045, 180046, 180047, 180048, 180049, 180050, 180051, 180052 were selected from the ORFs having the nucleotide sequence represented by SEQ ID NO: 173622.

The probes having the nucleotide sequence represented by SEQ ID NOS: 180053, 180054, 180055, 180056, 180057, 180058, 180059, 180060, 180061, 180062, 180063 were selected from the ORFs having the nucleotide sequence represented by SEQ ID NO: 173623.

The probes having the nucleotide sequence represented by SEQ ID NOS: 180064, 180065, 180066, 180067, 180068, 180069, 180070, 180071, 180072, 180073, 180074 were selected from the ORFs having the nucleotide sequence represented by SEQ ID NO: 173624.

The probes having the nucleotide sequence represented by SEQ ID NOS: 180075, 180076, 180077, 180078, 180079, 180080, 180081, 180082, 180083, 180084, 180085 were selected from the ORFs having the nucleotide sequence represented by SEQ ID NO: 173625.

The probes having the nucleotide sequence represented by SEQ ID NOS: 180086, 180087, 180088, 180089, 180090, 180091, 180092, 180093, 180094 were selected from the ORFs having the nucleotide sequence represented by SEQ ID NO: 173626.

The probes having the nucleotide sequence represented by SEQ ID NOS: 180095, 180096, 180097, 180098, 180099, 180100, 180101, 180102, 180103, 180104, 180105 were selected from the ORFs having the nucleotide sequence represented by SEQ ID NO: 173627.

The probes having the nucleotide sequence represented by SEQ ID NOS: 180106, 180107, 180108, 180109, 180110, 180111, 180112, 180113, 180114, 180115, 180116 were selected from the ORFs having the nucleotide sequence represented by SEQ ID NO: 173628.

The probes having the nucleotide sequence represented by SEQ ID NOS: 180117, 180118, 180119, 180120, 180121, 180122, 180123, 180124, 180125, 180126, 180127 were selected from the ORFs having the nucleotide sequence represented by SEQ ID NO: 173629.

The probes having the nucleotide sequence represented by SEQ ID NOS: 180128, 180129, 180130, 180131, 180132, 180133, 180134, 180135, 180136, 180137, 180138 were selected from the ORFs having the nucleotide sequence represented by SEQ ID NO: 173630.

The probes having the nucleotide sequence represented by SEQ ID NOS: 180139, 180140, 180141, 180142, 180143, 180144, 180145, 180146, 180147, 180148, 180149 were selected from the ORFs having the nucleotide sequence represented by SEQ ID NO: 173631.

The probes having the nucleotide sequence represented by SEQ ID NOS: 180150, 180151, 180152, 180153, 180154, 180155, 180156, 180157, 180158, 180159, 180160 were selected from the ORFs having the nucleotide sequence represented by SEQ ID NO: 173632.

The probes having the nucleotide sequence represented by SEQ ID NOS: 180161, 180162, 180163, 180164, 180165, 180166, 180167, 180168, 180169, 180170, 180171 were selected from the ORFs having the nucleotide sequence represented by SEQ ID NO: 173633.

The probes having the nucleotide sequence represented by SEQ ID NOS: 180172, 180173, 180174, 180175, 180176, 180177, 180178, 180179, 180180, 180181, 180182 were selected from the ORFs having the nucleotide sequence represented by SEQ ID NO: 173634.

The probes having the nucleotide sequence represented by SEQ ID NOS: 180183, 180184, 180185, 180186, 180187, 180188, 180189, 180190, 180191, 180192, 180193 were selected from the ORFs having the nucleotide sequence represented by SEQ ID NO: 173635.

The probes having the nucleotide sequence represented by SEQ ID NOS: 180194, 180195, 180196, 180197, 180198, 180199, 180200, 180201, 180202, 180203, 180204 were selected from the ORFs having the nucleotide sequence represented by SEQ ID NO: 173636.

The probes having the nucleotide sequence represented by SEQ ID NOS: 180205, 180206, 180207, 180208, 180209, 180210, 180211, 180212, 180213, 180214, 180215 were selected from the ORFs having the nucleotide sequence represented by SEQ ID NO: 173637.

The probes having the nucleotide sequence represented by SEQ ID NOS: 180216, 180217, 180218, 180219, 180220, 180221, 180222, 180223, 180224, 180225, 180226 were selected from the ORFs having the nucleotide sequence represented by SEQ ID NO: 173638.

The probes having the nucleotide sequence represented by SEQ ID NOS: 180227, 180228, 180229, 180230, 180231, 180232, 180233, 180234, 180235, 180236, 180237 were selected from the ORFs having the nucleotide sequence represented by SEQ ID NO: 173639.

The probes having the nucleotide sequence represented by SEQ ID NOS: 180238, 180239, 180240, 180241, 180242, 180243, 180244, 180245, 180246, 180247, 180248 were selected from the ORFs having the nucleotide sequence represented by SEQ ID NO: 173640.

The probes having the nucleotide sequence represented by SEQ ID NOS: 180249, 180250, 180251, 180252, 180253, 180254, 180255, 180256, 180257, 180258, 180259 were selected from the ORFs having the nucleotide sequence represented by SEQ ID NO: 173641.

The probes having the nucleotide sequence represented by SEQ ID NOS: 180260, 180261, 180262, 180263, 180264, 180265, 180266, 180267, 180268, 180269, 180270 were selected from the ORFs having the nucleotide sequence represented by SEQ ID NO: 173642.

The probes having the nucleotide sequence represented by SEQ ID NOS: 180271, 180272, 180273, 180274, 180275, 180276, 180277, 180278, 180279, 180280, 180281 were selected from the ORFs having the nucleotide sequence represented by SEQ ID NO: 173643.

The probes having the nucleotide sequence represented by SEQ ID NOS: 180282, 180283, 180284, 180285, 180286, 180287, 180288, 180289, 180290, 180291, 180292 were selected from the ORFs having the nucleotide sequence represented by SEQ ID NO: 173644.

The probes having the nucleotide sequence represented by SEQ ID NOS: 180293, 180294, 180295, 180296, 180297, 180298, 180299, 180300, 180301, 180302, 180303 were selected from the ORFs having the nucleotide sequence represented by SEQ ID NO: 173645.

The probes having the nucleotide sequence represented by SEQ ID NOS: 180304, 180305, 180306, 180307, 180308, 180309, 180310, 180311 were selected from the ORFs having the nucleotide sequence represented by SEQ ID NO: 173646.

The probes having the nucleotide sequence represented by SEQ ID NOS: 180312, 180313, 180314, 180315, 180316, 180317, 180318, 180319, 180320, 180321, 180322 were selected from the ORFs having the nucleotide sequence represented by SEQ ID NO: 173647.

The probes having the nucleotide sequence represented by SEQ ID NOS: 180323, 180324, 180325, 180326, 180327, 180328, 180329, 180330, 180331, 180332, 180333 were selected from the ORFs having the nucleotide sequence represented by SEQ ID NO: 173648.

The probes having the nucleotide sequence represented by SEQ ID NOS: 180334, 180335, 180336, 180337, 180338, 180339, 180340, 180341, 180342, 180343, 180344 were selected from the ORFs having the nucleotide sequence represented by SEQ ID NO: 173649.

The probes having the nucleotide sequence represented by SEQ ID NOS: 180345, 180346, 180347, 180348, 180349, 180350, 180351, 180352, 180353, 180354, 180355 were selected from the ORFs having the nucleotide sequence represented by SEQ ID NO: 173650.

The probes having the nucleotide sequence represented by SEQ ID NOS: 180356, 180357, 180358, 180359, 180360, 180361, 180362, 180363, 180364, 180365, 180366 were selected from the ORFs having the nucleotide sequence represented by SEQ ID NO: 173651.

The probes having the nucleotide sequence represented by SEQ ID NOS: 180367, 180368, 180369, 180370, 180371, 180372, 180373, 180374, 180375, 180376, 180377 were selected from the ORFs having the nucleotide sequence represented by SEQ ID NO: 173652.

The probes having the nucleotide sequence represented by SEQ ID NOS: 180378, 180379, 180380, 180381, 180382, 180383, 180384, 180385, 180386, 180387, 180388 were selected from the ORFs having the nucleotide sequence represented by SEQ ID NO: 173653.

The probes having the nucleotide sequence represented by SEQ ID NOS: 180389, 180390, 180391, 180392, 180393, 180394, 180395, 180396, 180397, 180398, 180399 were selected from the ORFs having the nucleotide sequence represented by SEQ ID NO: 173654.

The probes having the nucleotide sequence represented by SEQ ID NOS: 180400, 180401, 180402, 180403, 180404, 180405, 180406, 180407, 180408, 180409, 180410 were selected from the ORFs having the nucleotide sequence represented by SEQ ID NO: 173655.

The probes having the nucleotide sequence represented by SEQ ID NOS: 180411, 180412, 180413, 180414, 180415, 180416, 180417, 180418, 180419, 180420, 180421 were selected from the ORFs having the nucleotide sequence represented by SEQ ID NO: 173656.

The probes having the nucleotide sequence represented by SEQ ID NOS: 180422, 180423, 180424, 180425, 180426, 180427, 180428, 180429, 180430, 180431, 180432 were selected from the ORFs having the nucleotide sequence represented by SEQ ID NO: 173657.

The probes having the nucleotide sequence represented by SEQ ID NOS: 180433, 180434, 180435, 180436, 180437, 180438, 180439, 180440, 180441, 180442, 180443 were selected from the ORFs having the nucleotide sequence represented by SEQ ID NO: 173658.

The probes having the nucleotide sequence represented by SEQ ID NOS: 180444, 180445, 180446, 180447, 180448, 180449, 180450, 180451, 180452, 180453, 180454 were selected from the ORFs having the nucleotide sequence represented by SEQ ID NO: 173659.

The probes having the nucleotide sequence represented by SEQ ID NOS: 180455, 180456, 180457, 180458, 180459, 180460, 180461, 180462, 180463, 180464, 180465 were selected from the ORFs having the nucleotide sequence represented by SEQ ID NO: 173660.

The probes having the nucleotide sequence represented by SEQ ID NOS: 180466, 180467, 180468, 180469, 180470, 180471, 180472, 180473, 180474, 180475, 180476 were selected from the ORFs having the nucleotide sequence represented by SEQ ID NO: 173661.

The probes having the nucleotide sequence represented by SEQ ID NOS: 180477, 180478, 180479, 180480, 180481, 180482, 180483, 180484, 180485, 180486, 180487 were selected from the ORFs having the nucleotide sequence represented by SEQ ID NO: 173662.

The probes having the nucleotide sequence represented by SEQ ID NOS: 180488, 180489, 180490, 180491, 180492, 180493, 180494, 180495, 180496, 180497, 180498 were selected from the ORFs having the nucleotide sequence represented by SEQ ID NO: 173663.

The probes having the nucleotide sequence represented by SEQ ID NOS: 180499, 180500, 180501, 180502, 180503, 180504, 180505, 180506, 180507, 180508, 180509 were selected from the ORFs having the nucleotide sequence represented by SEQ ID NO: 173664.

The probes having the nucleotide sequence represented by SEQ ID NOS: 180510, 180511, 180512, 180513, 180514, 180515, 180516, 180517, 180518, 180519, 180520 were selected from the ORFs having the nucleotide sequence represented by SEQ ID NO: 173665.

The probes having the nucleotide sequence represented by SEQ ID NOS: 180521, 180522, 180523, 180524, 180525, 180526, 180527, 180528, 180529, 180530, 180531 were selected from the ORFs having the nucleotide sequence represented by SEQ ID NO: 173666.

The probes having the nucleotide sequence represented by SEQ ID NOS: 180532, 180533, 180534, 180535, 180536, 180537, 180538, 180539, 180540, 180541, 180542 were selected from the ORFs having the nucleotide sequence represented by SEQ ID NO: 173667.

The probes having the nucleotide sequence represented by SEQ ID NOS: 180543, 180544, 180545, 180546, 180547, 180548, 180549, 180550, 180551, 180552, 180553 were selected from the ORFs having the nucleotide sequence represented by SEQ ID NO: 173668.

The probes having the nucleotide sequence represented by SEQ ID NOS: 180554, 180555, 180556, 180557, 180558, 180559, 180560, 180561, 180562, 180563, 180564 were selected from the ORFs having the nucleotide sequence represented by SEQ ID NO: 173669.

The probes having the nucleotide sequence represented by SEQ ID NOS: 180565, 180566, 180567, 180568, 180569, 180570, 180571, 180572, 180573, 180574, 180575 were selected from the ORFs having the nucleotide sequence represented by SEQ ID NO: 173670.

The probes having the nucleotide sequence represented by SEQ ID NOS: 180576, 180577, 180578, 180579, 180580, 180581, 180582, 180583, 180584, 180585, 180586 were selected from the ORFs having the nucleotide sequence represented by SEQ ID NO: 173671.

The probes having the nucleotide sequence represented by SEQ ID NOS: 180587, 180588, 180589, 180590, 180591, 180592, 180593, 180594, 180595, 180596, 180597 were selected from the ORFs having the nucleotide sequence represented by SEQ ID NO: 173672.

The probes having the nucleotide sequence represented by SEQ ID NOS: 180598, 180599, 180600, 180601, 180602, 180603, 180604, 180605, 180606, 180607, 180608 were selected from the ORFs having the nucleotide sequence represented by SEQ ID NO: 173673.

The probes having the nucleotide sequence represented by SEQ ID NOS: 180609, 180610, 180611, 180612, 180613, 180614, 180615, 180616, 180617, 180618, 180619 were selected from the ORFs having the nucleotide sequence represented by SEQ ID NO: 173674.

The probes having the nucleotide sequence represented by SEQ ID NOS: 180620, 180621, 180622, 180623, 180624, 180625, 180626, 180627, 180628, 180629, 180630 were selected from the ORFs having the nucleotide sequence represented by SEQ ID NO: 173675.

The probes having the nucleotide sequence represented by SEQ ID NOS: 180631, 180632, 180633, 180634, 180635, 180636, 180637, 180638, 180639, 180640, 180641 were selected from the ORFs having the nucleotide sequence represented by SEQ ID NO: 173676.

The probes having the nucleotide sequence represented by SEQ ID NOS: 180642, 180643, 180644, 180645, 180646, 180647, 180648, 180649, 180650, 180651, 180652 were selected from the ORFs having the nucleotide sequence represented by SEQ ID NO: 173677.

The probes having the nucleotide sequence represented by SEQ ID NOS: 180653, 180654, 180655, 180656, 180657, 180658, 180659, 180660, 180661, 180662, 180663 were selected from the ORFs having the nucleotide sequence represented by SEQ ID NO: 173678.

The probes having the nucleotide sequence represented by SEQ ID NOS: 180664, 180665, 180666, 180667, 180668, 180669, 180670, 180671 were selected from the ORFs having the nucleotide sequence represented by SEQ ID NO: 173679.

The probes having the nucleotide sequence represented by SEQ ID NOS: 180672, 180673, 180674, 180675, 180676, 180677, 180678, 180679, 180680, 180681, 180682 were selected from the ORFs having the nucleotide sequence represented by SEQ ID NO: 173680.

The probes having the nucleotide sequence represented by SEQ ID NOS: 180683, 180684, 180685, 180686, 180687, 180688, 180689, 180690, 180691, 180692, 180693 were selected from the ORFs having the nucleotide sequence represented by SEQ ID NO: 173681.

The probes having the nucleotide sequence represented by SEQ ID NOS: 180694, 180695, 180696, 180697, 180698, 180699, 180700, 180701, 180702, 180703, 180704 were selected from the ORFs having the nucleotide sequence represented by SEQ ID NO: 173682.

The probes having the nucleotide sequence represented by SEQ ID NOS: 180705, 180706, 180707, 180708, 180709, 180710, 180711, 180712, 180713, 180714, 180715 were selected from the ORFs having the nucleotide sequence represented by SEQ ID NO: 173683.

The probes having the nucleotide sequence represented by SEQ ID NOS: 180716, 180717, 180718, 180719, 180720, 180721, 180722, 180723, 180724, 180725, 180726 were selected from the ORFs having the nucleotide sequence represented by SEQ ID NO: 173684.

The probes having the nucleotide sequence represented by SEQ ID NOS: 180727, 180728, 180729, 180730, 180731, 180732, 180733, 180734, 180735, 180736, 180737 were selected from the ORFs having the nucleotide sequence represented by SEQ ID NO: 173685.

The probes having the nucleotide sequence represented by SEQ ID NOS: 180738, 180739, 180740, 180741, 180742, 180743, 180744, 180745, 180746, 180747, 180748 were selected from the ORFs having the nucleotide sequence represented by SEQ ID NO: 173686.

The probes having the nucleotide sequence represented by SEQ ID NOS: 180749, 180750, 180751, 180752, 180753, 180754, 180755, 180756, 180757, 180758, 180759 were selected from the ORFs having the nucleotide sequence represented by SEQ ID NO: 173687.

The probes having the nucleotide sequence represented by SEQ ID NOS: 180760, 180761, 180762, 180763, 180764, 180765, 180766, 180767, 180768, 180769, 180770 were selected from the ORFs having the nucleotide sequence represented by SEQ ID NO: 173688.

The probes having the nucleotide sequence represented by SEQ ID NOS: 180771, 180772, 180773, 180774, 180775, 180776, 180777, 180778, 180779, 180780, 180781 were selected from the ORFs having the nucleotide sequence represented by SEQ ID NO: 173689.

The probes having the nucleotide sequence represented by SEQ ID NOS: 180782, 180783, 180784, 180785, 180786, 180787, 180788, 180789, 180790, 180791, 180792 were selected from the ORFs having the nucleotide sequence represented by SEQ ID NO: 173690.

The probes having the nucleotide sequence represented by SEQ ID NOS: 180793, 180794, 180795, 180796, 180797, 180798, 180799, 180800, 180801, 180802, 180803 were selected from the ORFs having the nucleotide sequence represented by SEQ ID NO: 173691.

The probes having the nucleotide sequence represented by SEQ ID NOS: 180804, 180805, 180806, 180807, 180808, 180809, 180810, 180811, 180812, 180813, 180814 were selected from the ORFs having the nucleotide sequence represented by SEQ ID NO: 173692.

The probes having the nucleotide sequence represented by SEQ ID NOS: 180815, 180816, 180817, 180818, 180819, 180820, 180821, 180822, 180823, 180824, 180825 were selected from the ORFs having the nucleotide sequence represented by SEQ ID NO: 173693.

The probes having the nucleotide sequence represented by SEQ ID NOS: 180826, 180827, 180828, 180829, 180830, 180831, 180832, 180833, 180834, 180835, 180836 were selected from the ORFs having the nucleotide sequence represented by SEQ ID NO: 173694.

The probes having the nucleotide sequence represented by SEQ ID NOS: 180837, 180838, 180839, 180840, 180841, 180842, 180843, 180844, 180845, 180846 were selected from the ORFs having the nucleotide sequence represented by SEQ ID NO: 173695.

The probes having the nucleotide sequence represented by SEQ ID NOS: 180847, 180848, 180849, 180850, 180851, 180852, 186853, 180854, 180855, 180856, 180857 were selected from the ORFs having the nucleotide sequence represented by SEQ ID NO: 173696.

The probes having the nucleotide sequence represented by SEQ ID NOS: 180858, 180859, 180860, 180861, 180862, 180863, 180864, 180865, 180866, 180867, 180868 were selected from the ORFs having the nucleotide sequence represented by SEQ ID NO: 173697.

The probes having the nucleotide sequence represented by SEQ ID NOS: 180869, 180870, 180871, 180872, 180873, 180874, 180875, 180876, 180877, 180878, 180879 were selected from the ORFs having the nucleotide sequence represented by SEQ ID NO: 173698.

The probes having the nucleotide sequence represented by SEQ ID NOS: 180880, 180881, 180882, 180883, 180884, 180885, 180886, 180887, 180888, 180889, 180890 were selected from the ORFs having the nucleotide sequence represented by SEQ ID NO: 173699.

The probes having the nucleotide sequence represented by SEQ ID NOS: 180891, 180892, 180893, 180894, 180895, 180896, 180897, 180898, 180899, 180900, 180901 were selected from the ORFs having the nucleotide sequence represented by SEQ ID NO: 173700.

The probes having the nucleotide sequence represented by SEQ ID NOS: 180902, 180903, 180904, 180905, 180906, 180907, 180908, 180909, 180910, 180911, 180912 were selected from the ORFs having the nucleotide sequence represented by SEQ ID NO: 173701.

The probes having the nucleotide sequence represented by SEQ ID NOS: 180913, 180914, 180915, 180916, 180917, 180918, 180919, 180920, 180921, 180922, 180923 were selected from the ORFs having the nucleotide sequence represented by SEQ ID NO: 173702.

The probes having the nucleotide sequence represented by SEQ ID NOS: 180924, 180925, 180926, 180927, 180928, 180929, 180930, 180931, 180932, 180933, 180934 were selected from the ORFs having the nucleotide sequence represented by SEQ ID NO: 173703.

The probes having the nucleotide sequence represented by SEQ ID NOS: 180935, 180936, 180937, 180938, 180939, 180940, 180941, 180942, 180943, 180944, 180945 were selected from the ORFs having the nucleotide sequence represented by SEQ ID NO: 173704.

The probes having the nucleotide sequence represented by SEQ ID NOS: 180946, 180947, 180948, 180949, 180950, 180951, 180952, 180953, 180954, 180955, 180956 were selected from the ORFs having the nucleotide sequence represented by SEQ ID NO: 173705.

The probes having the nucleotide sequence represented by SEQ ID NOS: 180957, 180958, 180959, 180960, 180961, 180962, 180963, 180964, 180965, 180966, 180967 were selected from the ORFs having the nucleotide sequence represented by SEQ ID NO: 173706.

The probes having the nucleotide sequence represented by SEQ ID NOS: 180968, 180969, 180970, 180971, 180972, 180973, 180974, 180975, 180976, 180977, 180978 were selected from the ORFs having the nucleotide sequence represented by SEQ ID NO: 173707.

The probes having the nucleotide sequence represented by SEQ ID NOS: 180979, 180980, 180981, 180982, 180983, 180984, 180985, 180986, 180987, 180988, 180989 were selected from the ORFs having the nucleotide sequence represented by SEQ ID NO: 173708.

The probes having the nucleotide sequence represented by SEQ ID NOS: 180990, 180991, 180992, 180993, 180994, 180995, 180996, 180997, 180998, 180999, 181000 were selected from the ORFs having the nucleotide sequence represented by SEQ ID NO: 173709.

The probes having the nucleotide sequence represented by SEQ ID NOS: 181001, 181002, 181003, 181004, 181005, 181006, 181007, 181008, 181009, 181010, 181011 were selected from the ORFs having the nucleotide sequence represented by SEQ ID NO: 173710.

The probes having the nucleotide sequence represented by SEQ ID NOS: 181012, 181013, 181014, 181015, 181016, 181017, 181018, 181019, 181020, 181021, 181022 were selected from the ORFs having the nucleotide sequence represented by SEQ ID NO: 173711.

The probes having the nucleotide sequence represented by SEQ ID NOS: 181023, 181024, 181025, 181026, 181027, 181028, 181029, 181030, 181031, 181032, 181033 were selected from the ORFs having the nucleotide sequence represented by SEQ ID NO: 173712.

The probes having the nucleotide sequence represented by SEQ ID NOS: 181034, 181035, 181036, 181037, 181038, 181039, 181040, 181041, 181042, 181043, 181044 were selected from the ORFs having the nucleotide sequence represented by SEQ ID NO: 173713.

The probes having the nucleotide sequence represented by SEQ ID NOS: 181045, 181046, 181047, 181048, 181049, 181050, 181051, 181052, 181053, 181054, 181055 were selected from the ORFs having the nucleotide sequence represented by SEQ ID NO: 173714.

The probes having the nucleotide sequence represented by SEQ ID NOS: 181056, 181057, 181058, 181059, 181060, 181061, 181062, 181063, 181064, 181065, 181066 were selected from the ORFs having the nucleotide sequence represented by SEQ ID NO: 173715.

The probes having the nucleotide sequence represented by SEQ ID NOS: 181067, 181068, 181069, 181070, 181071, 181072, 181073, 181074, 181075, 181076, 181077 were selected from the ORFs having the nucleotide sequence represented by SEQ ID NO: 173716.

The probes having the nucleotide sequence represented by SEQ ID NOS: 181078, 181079, 181080, 181081, 181082, 181083, 181084, 181085, 181086, 181087, 181088 were selected from the ORFs having the nucleotide sequence represented by SEQ ID NO: 173717.

The probes having the nucleotide sequence represented by SEQ ID NOS: 181089, 181090, 181091, 181092, 181093, 181094, 181095, 181096, 181097, 181098, 181099 were selected from the ORFs having the nucleotide sequence represented by SEQ ID NO: 173718.

The probes having the nucleotide sequence represented by SEQ ID NOS: 181100, 181101, 181102, 181103, 181104, 181105, 181106, 181107, 181108, 181109, 181110 were selected from the ORFs having the nucleotide sequence represented by SEQ ID NO: 173719.

The probes having the nucleotide sequence represented by SEQ ID NOS: 181111, 181112, 181113, 181114, 181115, 181116, 181117, 181118, 181119, 181120, 181121 were selected from the ORFs having the nucleotide sequence represented by SEQ ID NO: 173720.

The probes having the nucleotide sequence represented by SEQ ID NOS: 181122, 181123, 181124, 181125, 181126, 181127, 181128, 181129, 181130, 181131, 181132 were selected from the ORFs having the nucleotide sequence represented by SEQ ID NO: 173721.

The probes having the nucleotide sequence represented by SEQ ID NOS: 181133, 181134, 181135, 181136, 181137, 181138, 181139, 181140, 181141, 181142, 181143 were selected from the ORFs having the nucleotide sequence represented by SEQ ID NO: 173722.

The probes having the nucleotide sequence represented by SEQ ID NOS: 181144, 181145, 181146, 181147, 181148, 181149, 181150, 181151, 181152, 181153, 181154 were selected from the ORFs having the nucleotide sequence represented by SEQ ID NO: 173723.

The probes having the nucleotide sequence represented by SEQ ID NOS: 181155, 181156, 181157, 181158, 181159, 181160, 181161, 181162, 181163, 181164, 181165 were selected from the ORFs having the nucleotide sequence represented by SEQ ID NO: 173724.

The probes having the nucleotide sequence represented by SEQ ID NOS: 181166, 181167, 181168, 181169, 181170, 181171, 181172, 181173, 181174, 181175, 181176 were selected from the ORFs having the nucleotide sequence represented by SEQ ID NO: 173725.

The probes having the nucleotide sequence represented by SEQ ID NOS: 181177, 181178, 181179, 181180, 181181, 181182, 181183, 181184, 181185, 181186, 181187 were selected from the ORFs having the nucleotide sequence represented by SEQ ID NO: 173726.

The probes having the nucleotide sequence represented by SEQ ID NOS: 181188, 181189, 181190, 181191, 181192, 181193, 181194, 181195, 181196, 181197, 181198 were selected from the ORFs having the nucleotide sequence represented by SEQ ID NO: 173727.

The probes having the nucleotide sequence represented by SEQ ID NOS: 181199, 181200, 181201, 181202, 181203, 181204, 181205, 181206, 181207, 181208, 181209 were selected from the ORFs having the nucleotide sequence represented by SEQ ID NO: 173728.

The probes having the nucleotide sequence represented by SEQ ID NOS: 181210, 181211, 181212, 181213, 181214, 181215, 181216, 181217, 181218, 181219, 181220 were selected from the ORFs having the nucleotide sequence represented by SEQ ID NO: 173729.

The probes having the nucleotide sequence represented by SEQ ID NOS: 181221, 181222, 181223, 181224, 181225, 181226, 181227, 181228, 181229, 181230, 181231 were selected from the ORFs having the nucleotide sequence represented by SEQ ID NO: 173730.

The probes having the nucleotide sequence represented by SEQ ID NOS: 181232, 181233, 181234, 181235, 181236, 181237, 181238, 181239, 181240, 181241, 181242 were selected from the ORFs having the nucleotide sequence represented by SEQ ID NO: 173731.

The probes having the nucleotide sequence represented by SEQ ID NOS: 181243, 181244, 181245, 181246, 181247, 181248, 181249, 181250, 181251, 181252, 181253 were selected from the ORFs having the nucleotide sequence represented by SEQ ID NO: 173732.

The probes having the nucleotide sequence represented by SEQ ID NOS: 181254, 181255, 181256, 181257, 181258, 181259, 181260, 181261, 181262, 181263, 181264 were selected from the ORFs having the nucleotide sequence represented by SEQ ID NO: 173733.

The probes having the nucleotide sequence represented by SEQ ID NOS: 181265, 181266, 181267, 181268, 181269, 181270, 181271, 181272, 181273, 181274, 181275 were selected from the ORFs having the nucleotide sequence represented by SEQ ID NO: 173734.

The probes having the nucleotide sequence represented by SEQ ID NOS: 181276, 181277, 181278, 181279, 181280, 181281, 181282, 181283, 181284, 181285, 181286 were selected from the ORFs having the nucleotide sequence represented by SEQ ID NO: 173735.

The probes having the nucleotide sequence represented by SEQ ID NOS: 181287, 181288, 181289, 181290, 181291, 181292, 181293, 181294, 181295, 181296, 181297 were selected from the ORFs having the nucleotide sequence represented by SEQ ID NO: 173736.

The probes having the nucleotide sequence represented by SEQ ID NOS: 181298, 181299, 181300, 181301, 181302, 181303, 181304, 181305, 181306, 181307, 181308 were selected from the ORFs having the nucleotide sequence represented by SEQ ID NO: 173737.

The probes having the nucleotide sequence represented by SEQ ID NOS: 181309, 181310, 181311, 181312, 181313, 181314, 181315, 181316, 181317, 181318, 181319 were selected from the ORFs having the nucleotide sequence represented by SEQ ID NO: 173738.

The probes having the nucleotide sequence represented by SEQ ID NOS: 181320, 181321, 181322, 181323, 181324, 181325, 181326, 181327, 181328, 181329, 181330 were selected from the ORFs having the nucleotide sequence represented by SEQ ID NO: 173739.

The probes having the nucleotide sequence represented by SEQ ID NOS: 181331, 181332, 181333, 181334, 181335, 181336, 181337, 181338, 181339, 181340, 181341 were selected from the ORFs having the nucleotide sequence represented by SEQ ID NO: 173740.

The probes having the nucleotide sequence represented by SEQ ID NOS: 181342, 181343, 181344, 181345, 181346, 181347, 181348, 181349, 181350, 181351, 181352 were selected from the ORFs having the nucleotide sequence represented by SEQ ID NO: 173741.

The probes having the nucleotide sequence represented by SEQ ID NOS: 181353, 181354, 181355, 181356, 181357, 181358, 181359, 181360, 181361, 181362, 181363 were selected from the ORFs having the nucleotide sequence represented by SEQ ID NO: 173742.

The probes having the nucleotide sequence represented by SEQ ID NOS: 181364, 181365, 181366, 181367, 181368, 181369, 181370, 181371, 181372, 181373, 181374 were selected from the ORFs having the nucleotide sequence represented by SEQ ID NO: 173743.

The probes having the nucleotide sequence represented by SEQ ID NOS: 181375, 181376, 181377, 181378, 181379, 181380, 181381, 181382, 181383, 181384, 181385 were selected from the ORFs having the nucleotide sequence represented by SEQ ID NO: 173744.

The probes having the nucleotide sequence represented by SEQ ID NOS: 181386, 181387, 181388, 181389, 181390, 181391, 181392, 181393, 181394, 181395, 181396 were selected from the ORFs having the nucleotide sequence represented by SEQ ID NO: 173745.

The probes having the nucleotide sequence represented by SEQ ID NOS: 181397, 181398, 181399, 181400, 181401, 181402, 181403, 181404, 181405, 181406, 181407 were selected from the ORFs having the nucleotide sequence represented by SEQ ID NO: 173746.

The probes having the nucleotide sequence represented by SEQ ID NOS: 181408, 181409, 181410, 181411, 181412, 181413, 181414, 181415, 181416, 181417, 181418 were selected from the ORFs having the nucleotide sequence represented by SEQ ID NO: 173747.

The probes having the nucleotide sequence represented by SEQ ID NOS: 181419, 181420, 181421, 181422, 181423, 181424, 181425, 181426, 181427, 181428, 181429 were selected from the ORFs having the nucleotide sequence represented by SEQ ID NO: 173748.

The probes having the nucleotide sequence represented by SEQ ID NOS: 181430, 181431, 181432, 181433, 181434, 181435, 181436, 181437, 181438, 181439, 181440 were selected from the ORFs having the nucleotide sequence represented by SEQ ID NO: 173749.

The probes having the nucleotide sequence represented by SEQ ID NOS: 181441, 181442, 181443, 181444, 181445, 181446, 181447, 181448, 181449, 181450, 181451 were selected from the ORFs having the nucleotide sequence represented by SEQ ID NO: 173750.

The probes having the nucleotide sequence represented by SEQ ID NOS: 181452, 181453, 181454, 181455, 181456, 181457, 181458, 181459, 181460, 181461, 181462 were selected from the ORFs having the nucleotide sequence represented by SEQ ID NO: 173751.

The probes having the nucleotide sequence represented by SEQ ID NOS: 181463, 181464, 181465, 181466, 181467, 181468, 181469, 181470, 181471, 181472, 181473 were selected from the ORFs having the nucleotide sequence represented by SEQ ID NO: 173752.

The probes having the nucleotide sequence represented by SEQ ID NOS: 181474, 181475, 181476, 181477, 181478, 181479, 181480, 181481, 181482, 181483, 181484 were selected from the ORFs having the nucleotide sequence represented by SEQ ID NO: 173753.

The probes having the nucleotide sequence represented by SEQ ID NOS: 181485, 181486, 181487, 181488, 181489, 181490, 181491, 181492, 181493, 181494, 181495 were selected from the ORFs having the nucleotide sequence represented by SEQ ID NO: 173754.

The probes having the nucleotide sequence represented by SEQ ID NOS: 181496, 181497, 181498, 181499, 181500, 181501, 181502, 181503, 181504, 181505, 181506 were selected from the ORFs having the nucleotide sequence represented by SEQ ID NO: 173755.

The probes having the nucleotide sequence represented by SEQ ID NOS: 181507, 181508, 181509, 181510, 181511, 181512, 181513, 181514, 181515, 181516, 181517 were selected from the ORFs having the nucleotide sequence represented by SEQ ID NO: 173756.

The probes having the nucleotide sequence represented by SEQ ID NOS: 181518, 181519, 181520, 181521, 181522, 181523, 181524, 181525, 181526, 181527, 181528 were selected from the ORFs having the nucleotide sequence represented by SEQ ID NO: 173757.

The probes having the nucleotide sequence represented by SEQ ID NOS: 181529, 181530, 181531, 181532, 181533, 181534, 181535, 181536, 181537, 181538, 181539 were selected from the ORFs having the nucleotide sequence represented by SEQ ID NO: 173758.

The probes having the nucleotide sequence represented by SEQ ID NOS: 181540, 181541, 181542, 181543, 181544, 181545, 181546, 181547, 181548, 181549, 181550 were selected from the ORFs having the nucleotide sequence represented by SEQ ID NO: 173759.

The probes having the nucleotide sequence represented by SEQ ID NOS: 181551, 181552, 181553, 181554, 181555, 181556, 181557, 181558, 181559, 181560, 181561 were selected from the ORFs having the nucleotide sequence represented by SEQ ID NO: 173760.

The probes having the nucleotide sequence represented by SEQ ID NOS: 181562, 181563, 181564, 181565, 181566, 181567, 181568, 181569, 181570, 181571, 181572 were selected from the ORFs having the nucleotide sequence represented by SEQ ID NO: 173761.

The probes having the nucleotide sequence represented by SEQ ID NOS: 181573, 181574, 181575, 181576, 181577, 181578, 181579, 181580, 181581, 181582, 181583 were selected from the ORFs having the nucleotide sequence represented by SEQ ID NO: 173762.

The probes having the nucleotide sequence represented by SEQ ID NOS: 181584, 181585, 181586, 181587, 181588, 181589, 181590, 181591, 181592, 181593, 181594 were selected from the ORFs having the nucleotide sequence represented by SEQ ID NO: 173763.

The probes having the nucleotide sequence represented by SEQ ID NOS: 181595, 181596, 181597, 181598, 181599, 181600, 181601, 181602, 181603, 181604, 181605 were selected from the ORFs having the nucleotide sequence represented by SEQ ID NO: 173764.

The probes having the nucleotide sequence represented by SEQ ID NOS: 181606, 181607, 181608, 181609, 181610, 181611, 181612, 181613, 181614, 181615, 181616 were selected from the ORFs having the nucleotide sequence represented by SEQ ID NO: 173765.

The probes having the nucleotide sequence represented by SEQ ID NOS: 181617, 181618, 181619, 181620, 181621, 181622, 181623, 181624, 181625, 181626, 181627 were selected from the ORFs having the nucleotide sequence represented by SEQ ID NO: 173766.

The probes having the nucleotide sequence represented by SEQ ID NOS: 181628, 181629, 181630, 181631, 181632, 181633, 181634, 181635, 181636, 181637, 181638 were selected from the ORFs having the nucleotide sequence represented by SEQ ID NO: 173767.

The probes having the nucleotide sequence represented by SEQ ID NOS: 181639, 181640, 181641, 181642, 181643, 181644, 181645, 181646, 181647, 181648, 181649 were selected from the ORFs having the nucleotide sequence represented by SEQ ID NO: 173768.

The probes having the nucleotide sequence represented by SEQ ID NOS: 181650, 181651, 181652, 181653, 181654, 181655, 181656, 181657, 181658, 181659, 181660 were selected from the ORFs having the nucleotide sequence represented by SEQ ID NO: 173769.

The probes having the nucleotide sequence represented by SEQ ID NOS: 181661, 181662, 181663, 181664, 181665, 181666, 181667, 181668, 181669, 181670, 181671 were selected from the ORFs having the nucleotide sequence represented by SEQ ID NO: 173770.

The probes having the nucleotide sequence represented by SEQ ID NOS: 181672, 181673, 181674, 181675, 181676, 181677, 181678, 181679, 181680, 181681, 181682 were selected from the ORFs having the nucleotide sequence represented by SEQ ID NO: 173771.

The probes having the nucleotide sequence represented by SEQ ID NOS: 181683, 181684, 181685, 181686, 181687, 181688, 181689, 181690, 181691, 181692, 181693 were selected from the ORFs having the nucleotide sequence represented by SEQ ID NO: 173772.

The probes having the nucleotide sequence represented by SEQ ID NOS: 181694, 181695, 181696, 181697, 181698, 181699, 181700, 181701, 181702, 181703, 181704 were selected from the ORFs having the nucleotide sequence represented by SEQ ID NO: 173773.

The probes having the nucleotide sequence represented by SEQ ID NOS: 181705, 181706, 181707, 181708, 181709, 181710, 181711, 181712, 181713, 181714, 181715 were selected from the ORFs having the nucleotide sequence represented by SEQ ID NO: 173774.

The probes having the nucleotide sequence represented by SEQ ID NOS: 181716, 181717, 181718, 181719, 181720, 181721, 181722, 181723, 181724, 181725, 181726 were selected from the ORFs having the nucleotide sequence represented by SEQ ID NO: 173775.

The probes having the nucleotide sequence represented by SEQ ID NOS: 181727, 181728, 181729, 181730, 181731, 181732, 181733, 181734, 181735, 181736, 181737 were selected from the ORFs having the nucleotide sequence represented by SEQ ID NO: 173776.

The probes having the nucleotide sequence represented by SEQ ID NOS: 181738, 181739, 181740, 181741, 181742, 181743, 181744, 181745, 181746, 181747, 181748 were selected from the ORFs having the nucleotide sequence represented by SEQ ID NO: 173777.

The probes having the nucleotide sequence represented by SEQ ID NOS: 181749, 181750, 181751, 181752, 181753, 181754, 181755, 181756, 181757, 181758, 181759 were selected from the ORFs having the nucleotide sequence represented by SEQ ID NO: 173778.

The probes having the nucleotide sequence represented by SEQ ID NOS: 181760, 181761, 181762, 181763, 181764, 181765, 181766, 181767, 181768, 181769, 181770 were selected from the ORFs having the nucleotide sequence represented by SEQ ID NO: 173779.

The probes having the nucleotide sequence represented by SEQ ID NOS: 181771, 181772, 181773, 181774, 181775, 181776, 181777, 181778, 181779, 181780, 181781 were selected from the ORFs having the nucleotide sequence represented by SEQ ID NO: 173780.

The probes having the nucleotide sequence represented by SEQ ID NOS: 181782, 181783, 181784, 181785, 181786, 181787, 181788, 181789, 181790, 181791, 181792 were selected from the ORFs having the nucleotide sequence represented by SEQ ID NO: 173781.

The probes having the nucleotide sequence represented by SEQ ID NOS: 181793, 181794, 181795, 181796, 181797, 181798, 181799, 181800, 181801, 181802, 181803 were selected from the ORFs having the nucleotide sequence represented by SEQ ID NO: 173782.

The probes having the nucleotide sequence represented by SEQ ID NOS: 181804, 181805, 181806, 181807, 181808, 181809, 181810, 181811, 181812, 181813, 181814 were selected from the ORFs having the nucleotide sequence represented by SEQ ID NO: 173783.

The probes having the nucleotide sequence represented by SEQ ID NOS: 181815, 181816, 181817, 181818, 181819, 181820, 181821, 181822, 181823, 181824, 181825 were selected from the ORFs having the nucleotide sequence represented by SEQ ID NO: 173784.

The probes having the nucleotide sequence represented by SEQ ID NOS: 181826, 181827, 181828, 181829, 181830, 181831, 181832, 181833, 181834, 181835, 181836 were selected from the ORFs having the nucleotide sequence represented by SEQ ID NO: 173785.

The probes having the nucleotide sequence represented by SEQ ID NOS: 181837, 181838, 181839, 181840, 181841, 181842, 181843, 181844, 181845, 181846, 181847 were selected from the ORFs having the nucleotide sequence represented by SEQ ID NO: 173786.

The probes having the nucleotide sequence represented by SEQ ID NOS: 181848, 181849, 181850, 181851, 181852, 181853, 181854, 181855, 181856, 181857, 181858 were selected from the ORFs having the nucleotide sequence represented by SEQ ID NO: 173787.

The probes having the nucleotide sequence represented by SEQ ID NOS: 181859, 181860, 181861, 181862, 181863, 181864, 181865, 181866, 181867, 181868, 181869 were selected from the ORFs having the nucleotide sequence represented by SEQ ID NO: 173788.

The probes having the nucleotide sequence represented by SEQ ID NOS: 181870, 181871, 181872, 181873, 181874, 181875, 181876, 181877, 181878, 181879, 181880 were selected from the ORFs having the nucleotide sequence represented by SEQ ID NO: 173789.

The probes having the nucleotide sequence represented by SEQ ID NOS: 181881, 181882, 181883, 181884, 181885, 181886, 181887, 181888, 181889, 181890, 181891 were selected from the ORFs having the nucleotide sequence represented by SEQ ID NO: 173790.

The probes having the nucleotide sequence represented by SEQ ID NOS: 181892, 181893, 181894, 181895, 181896, 181897, 181898, 181899, 181900, 181901, 181902 were selected from the ORFs having the nucleotide sequence represented by SEQ ID NO: 173791.

The probes having the nucleotide sequence represented by SEQ ID NOS: 181903, 181904, 181905, 181906, 181907, 181908, 181909, 181910, 181911, 181912, 181913 were selected from the ORFs having the nucleotide sequence represented by SEQ ID NO: 173792.

The probes having the nucleotide sequence represented by SEQ ID NOS: 181914, 181915, 181916, 181917, 181918, 181919, 181920, 181921, 181922, 181923, 181924 were selected from the ORFs having the nucleotide sequence represented by SEQ ID NO: 173793.

The probes having the nucleotide sequence represented by SEQ ID NOS: 181925, 181926, 181927, 181928, 181929, 181930, 181931, 181932, 181933, 181934, 181935 were selected from the ORFs having the nucleotide sequence represented by SEQ ID NO: 173794.

The probes having the nucleotide sequence represented by SEQ ID NOS: 181936, 181937, 181938, 181939, 181940, 181941, 181942, 181943, 181944, 181945, 181946 were selected from the ORFs having the nucleotide sequence represented by SEQ ID NO: 173795.

The probes having the nucleotide sequence represented by SEQ ID NOS: 181947, 181948, 181949, 181950, 181951, 181952, 181953, 181954, 181955, 181956, 181957 were selected from the ORFs having the nucleotide sequence represented by SEQ ID NO: 173796.

The probes having the nucleotide sequence represented by SEQ ID NOS: 181958, 181959, 181960, 181961, 181962, 181963, 181964, 181965, 181966, 181967, 181968 were selected from the ORFs having the nucleotide sequence represented by SEQ ID NO: 173797.

The probes having the nucleotide sequence represented by SEQ ID NOS: 181969, 181970, 181971, 181972, 181973, 181974, 181975, 181976, 181977, 181978 were selected from the ORFs having the nucleotide sequence represented by SEQ ID NO: 173798.

The probes having the nucleotide sequence represented by SEQ ID NOS: 181979, 181980, 181981, 181982, 181983, 181984, 181985, 181986, 181987, 181988, 181989 were selected from the ORFs having the nucleotide sequence represented by SEQ ID NO: 173799.

The probes having the nucleotide sequence represented by SEQ ID NOS: 181990, 181991, 181992, 181993, 181994, 181995, 181996, 181997, 181998, 181999, 182000 were selected from the ORFs having the nucleotide sequence represented by SEQ ID NO: 173800.

The probes having the nucleotide sequence represented by SEQ ID NOS: 182001, 182002, 182003, 182004, 182005, 182006, 182007, 182008, 182009, 182010, 182011 were selected from the ORFs having the nucleotide sequence represented by SEQ ID NO: 173801.

The probes having the nucleotide sequence represented by SEQ ID NOS: 182012, 182013, 182014, 182015, 182016, 182017, 182018, 182019, 182020, 182021, 182022 were selected from the ORFs having the nucleotide sequence represented by SEQ ID NO: 173802.

The probes having the nucleotide sequence represented by SEQ ID NOS: 182023, 182024, 182025, 182026, 182027, 182028, 182029, 182030, 182031, 182032, 182033 were selected from the ORFs having the nucleotide sequence represented by SEQ ID NO: 173803.

The probes having the nucleotide sequence represented by SEQ ID NOS: 182034, 182035, 182036, 182037, 182038, 182039, 182040, 182041, 182042, 182043, 182044 were selected from the ORFs having the nucleotide sequence represented by SEQ ID NO: 173804.

The probes having the nucleotide sequence represented by SEQ ID NOS: 182045, 182046, 182047, 182048, 182049, 182050, 182051, 182052, 182053, 182054, 182055 were selected from the ORFs having the nucleotide sequence represented by SEQ ID NO: 173805.

The probes having the nucleotide sequence represented by SEQ ID NOS: 182056, 182057, 182058, 182059, 182060, 182061, 182062, 182063, 182064, 182065, 182066 were selected from the ORFs having the nucleotide sequence represented by SEQ ID NO: 173806.

The probes having the nucleotide sequence represented by SEQ ID NOS: 182067, 182068, 182069, 182070, 182071, 182072, 182073, 182074, 182075, 182076, 182077 were selected from the ORFs having the nucleotide sequence represented by SEQ ID NO: 173807.

The probes having the nucleotide sequence represented by SEQ ID NOS: 182078, 182079, 182080, 182081, 182082, 182083, 182084, 182085, 182086, 182087, 182088 were selected from the ORFs having the nucleotide sequence represented by SEQ ID NO: 173808.

The probes having the nucleotide sequence represented by SEQ ID NOS: 182089, 182090, 182091, 182092, 182093, 182094, 182095, 182096, 182097, 182098, 182099 were selected from the ORFs having the nucleotide sequence represented by SEQ ID NO: 173809.

The probes having the nucleotide sequence represented by SEQ ID NOS: 182100, 182101, 182102, 182103, 182104, 182105, 182106, 182107, 182108, 182109, 182110 were selected from the ORFs having the nucleotide sequence represented by SEQ ID NO: 173810.

The probes having the nucleotide sequence represented by SEQ ID NOS: 182111, 182112, 182113, 182114, 182115, 182116, 182117, 182118, 182119, 182120, 182121 were selected from the ORFs having the nucleotide sequence represented by SEQ ID NO: 173811.

The probes having the nucleotide sequence represented by SEQ ID NOS: 182122, 182123, 182124, 182125, 182126, 182127, 182128, 182129, 182130, 182131, 182132 were selected from the ORFs having the nucleotide sequence represented by SEQ ID NO: 173812.

The probes having the nucleotide sequence represented by SEQ ID NOS: 182133, 182134, 182135, 182136, 182137, 182138, 182139, 182140, 182141, 182142, 182143 were selected from the ORFs having the nucleotide sequence represented by SEQ ID NO: 173813.

The probes having the nucleotide sequence represented by SEQ ID NOS: 182144, 182145, 182146, 182147, 182148, 182149, 182150, 182151, 182152, 182153, 182154 were selected from the ORFs having the nucleotide sequence represented by SEQ ID NO: 173814.

The probes having the nucleotide sequence represented by SEQ ID NOS: 182155, 182156, 182157, 182158, 182159, 182160, 182161, 182162, 182163, 182164, 182165 were selected from the ORFs having the nucleotide sequence represented by SEQ ID NO: 173815.

The probes having the nucleotide sequence represented by SEQ ID NOS: 182166, 182167, 182168, 182169, 182170, 182171, 182172, 182173, 182174, 182175, 182176 were selected from the ORFs having the nucleotide sequence represented by SEQ ID NO: 173816.

The probes having the nucleotide sequence represented by SEQ ID NOS: 182177, 182178, 182179, 182180, 182181, 182182, 182183, 182184, 182185, 182186, 182187 were selected from the ORFs having the nucleotide sequence represented by SEQ ID NO: 173817.

The probes having the nucleotide sequence represented by SEQ ID NOS: 182188, 182189, 182190, 182191, 182192, 182193, 182194, 182195, 182196, 182197, 182198 were selected from the ORFs having the nucleotide sequence represented by SEQ ID NO: 173818.

The probes having the nucleotide sequence represented by SEQ ID NOS: 182199, 182200, 182201, 182202, 182203, 182204, 182205, 182206, 182207, 182208, 182209 were selected from the ORFs having the nucleotide sequence represented by SEQ ID NO: 173819.

The probes having the nucleotide sequence represented by SEQ ID NOS: 182210, 182211, 182212, 182213, 182214, 182215, 182216, 182217, 182218, 182219, 182220 were selected from the ORFs having the nucleotide sequence represented by SEQ ID NO: 173820.

The probes having the nucleotide sequence represented by SEQ ID NOS: 182221, 182222, 182223, 182224, 182225, 182226, 182227, 182228, 182229, 182230, 182231 were selected from the ORFs having the nucleotide sequence represented by SEQ ID NO: 173821.

The probes having the nucleotide sequence represented by SEQ ID NOS: 182232, 182233, 182234, 182235, 182236, 182237, 182238, 182239, 182240, 182241, 182242 were selected from the ORFs having the nucleotide sequence represented by SEQ ID NO: 173822.

The probes having the nucleotide sequence represented by SEQ ID NOS: 182243, 182244, 182245, 182246, 182247, 182248, 182249, 182250, 182251, 182252, 182253 were selected from the ORFs having the nucleotide sequence represented by SEQ ID NO: 173823.

The probes having the nucleotide sequence represented by SEQ ID NOS: 182254, 182255, 182256, 182257, 182258, 182259, 182260, 182261, 182262, 182263, 182264 were selected from the ORFs having the nucleotide sequence represented by SEQ ID NO: 173824.

The probes having the nucleotide sequence represented by SEQ ID NOS: 182265, 182266, 182267, 182268, 182269, 182270, 182271, 182272, 182273, 182274, 182275 were selected from the ORFs having the nucleotide sequence represented by SEQ ID NO: 173825.

The probes having the nucleotide sequence represented by SEQ ID NOS: 182276, 182277, 182278, 182279, 182280, 182281, 182282, 182283, 182284, 182285, 182286 were selected from the ORFs having the nucleotide sequence represented by SEQ ID NO: 173826.

The probes having the nucleotide sequence represented by SEQ ID NOS: 182287, 182288, 182289, 182290, 182291, 182292, 182293, 182294, 182295, 182296, 182297 were selected from the ORFs having the nucleotide sequence represented by SEQ ID NO: 173827.

The probes having the nucleotide sequence represented by SEQ ID NOS: 182298, 182299, 182300, 182301, 182302, 182303, 182304, 182305, 182306, 182307, 182308 were selected from the ORFs having the nucleotide sequence represented by SEQ ID NO: 173828.

The probes having the nucleotide sequence represented by SEQ ID NOS: 182309, 182310, 182311, 182312, 182313, 182314, 182315, 182316, 182317, 182318, 182319 were selected from the ORFs having the nucleotide sequence represented by SEQ ID NO: 173829.

The probes having the nucleotide sequence represented by SEQ ID NOS: 182320, 182321, 182322, 182323, 182324, 182325, 182326, 182327, 182328, 182329, 182330 were selected from the ORFs having the nucleotide sequence represented by SEQ ID NO: 173830.

The probes having the nucleotide sequence represented by SEQ ID NOS: 182331, 182332, 182333, 182334, 182335, 182336, 182337, 182338, 182339, 182340, 182341 were selected from the ORFs having the nucleotide sequence represented by SEQ ID NO: 173831.

The probes having the nucleotide sequence represented by SEQ ID NOS: 182342, 182343, 182344, 182345, 182346, 182347, 182348, 182349, 182350, 182351, 182352 were selected from the ORFs having the nucleotide sequence represented by SEQ ID NO: 173832.

The probes having the nucleotide sequence represented by SEQ ID NOS: 182353, 182354, 182355, 182356, 182357, 182358, 182359, 182360, 182361, 182362, 182363 were selected from the ORFs having the nucleotide sequence represented by SEQ ID NO: 173833.

The probes having the nucleotide sequence represented by SEQ ID NOS: 182364, 182365, 182366, 182367, 182368, 182369, 182370, 182371, 182372, 182373, 182374 were selected from the ORFs having the nucleotide sequence represented by SEQ ID NO: 173834.

The probes having the nucleotide sequence represented by SEQ ID NOS: 182375, 182376, 182377, 182378, 182379, 182380, 182381, 182382, 182383, 182384, 182385 were selected from the ORFs having the nucleotide sequence represented by SEQ ID NO: 173835.

The probes having the nucleotide sequence represented by SEQ ID NOS: 182386, 182387, 182388, 182389, 182390, 182391, 182392, 182393, 182394, 182395, 182396 were selected from the ORFs having the nucleotide sequence represented by SEQ ID NO: 173836.

The probes having the nucleotide sequence represented by SEQ ID NOS: 182397, 182398, 182399, 182400, 182401, 182402, 182403, 182404, 182405, 182406, 182407 were selected from the ORFs having the nucleotide sequence represented by SEQ ID NO: 173837.

The probes having the nucleotide sequence represented by SEQ ID NOS: 182408, 182409, 182410, 182411, 182412, 182413, 182414, 182415, 182416, 182417, 182418 were selected from the ORFs having the nucleotide sequence represented by SEQ ID NO: 173838.

The probes having the nucleotide sequence represented by SEQ ID NOS: 182419, 182420, 182421, 182422, 182423, 182424, 182425, 182426, 182427, 182428, 182429 were selected from the ORFs having the nucleotide sequence represented by SEQ ID NO: 173839.

The probes having the nucleotide sequence represented by SEQ ID NOS: 182430, 182431, 182432, 182433, 182434, 182435, 182436, 182437, 182438, 182439, 182440 were selected from the ORFs having the nucleotide sequence represented by SEQ ID NO: 173840.

The probes having the nucleotide sequence represented by SEQ ID NOS: 182441, 182442, 182443, 182444, 182445, 182446, 182447, 182448, 182449, 182450, 182451 were selected from the ORFs having the nucleotide sequence represented by SEQ ID NO: 173841.

The probes having the nucleotide sequence represented by SEQ ID NOS: 182452, 182453, 182454, 182455, 182456, 182457, 182458, 182459, 182460, 182461, 182462 were selected from the ORFs having the nucleotide sequence represented by SEQ ID NO: 173842.

The probes having the nucleotide sequence represented by SEQ ID NOS: 182463, 182464, 182465, 182466, 182467, 182468, 182469, 182470, 182471, 182472, 182473 were selected from the ORFs having the nucleotide sequence represented by SEQ ID NO: 173843.

The probes having the nucleotide sequence represented by SEQ ID NOS: 182474, 182475, 182476, 182477, 182478, 182479, 182480, 182481, 182482, 182483, 182484 were selected from the ORFs having the nucleotide sequence represented by SEQ ID NO: 173844.

The probes having the nucleotide sequence represented by SEQ ID NOS: 182485, 182486, 182487, 182488, 182489, 182490, 182491, 182492, 182493, 182494, 182495 were selected from the ORFs having the nucleotide sequence represented by SEQ ID NO: 173845.

The probes having the nucleotide sequence represented by SEQ ID NOS: 182496, 182497, 182498, 182499, 182500, 182501, 182502, 182503, 182504, 182505, 182506 were selected from the ORFs having the nucleotide sequence represented by SEQ ID NO: 173846.

The probes having the nucleotide sequence represented by SEQ ID NOS: 182507, 182508, 182509, 182510, 182511, 182512, 182513, 182514, 182515, 182516, 182517 were selected from the ORFs having the nucleotide sequence represented by SEQ ID NO: 173847.

The probes having the nucleotide sequence represented by SEQ ID NOS: 182518, 182519, 182520, 182521, 182522, 182523, 182524, 182525, 182526, 182527, 182528 were selected from the ORFs having the nucleotide sequence represented by SEQ ID NO: 173848.

The probes having the nucleotide sequence represented by SEQ ID NOS: 182529, 182530, 182531, 182532, 182533, 182534, 182535, 182536, 182537, 182538, 182539 were selected from the ORFs having the nucleotide sequence represented by SEQ ID NO: 173849.

The probes having the nucleotide sequence represented by SEQ ID NOS: 182540, 182541, 182542, 182543, 182544, 182545, 182546, 182547, 182548, 182549, 182550 were selected from the ORFs having the nucleotide sequence represented by SEQ ID NO: 173850.

The probes having the nucleotide sequence represented by SEQ ID NOS: 182551, 182552, 182553, 182554, 182555, 182556, 182557, 182558, 182559, 182560, 182561 were selected from the ORFs having the nucleotide sequence represented by SEQ ID NO: 173851.

The probes having the nucleotide sequence represented by SEQ ID NOS: 182562, 182563, 182564, 182565, 182566, 182567, 182568, 182569, 182570, 182571, 182572 were selected from the ORFs having the nucleotide sequence represented by SEQ ID NO: 173852.

The probes having the nucleotide sequence represented by SEQ ID NOS: 182573, 182574, 182575, 182576, 182577, 182578, 182579, 182580, 182581, 182582, 182583 were selected from the ORFs having the nucleotide sequence represented by SEQ ID NO: 173853.

The probes having the nucleotide sequence represented by SEQ ID NOS: 182584, 182585, 182586, 182587, 182588, 182589, 182590, 182591, 182592, 182593, 182594 were selected from the ORFs having the nucleotide sequence represented by SEQ ID NO: 173854.

The probes having the nucleotide sequence represented by SEQ ID NOS: 182595, 182596, 182597, 182598, 182599, 182600, 182601, 182602, 182603, 182604, 182605 were selected from the ORFs having the nucleotide sequence represented by SEQ ID NO: 173855.

The probes having the nucleotide sequence represented by SEQ ID NOS: 182606, 182607, 182608, 182609, 182610, 182611, 182612, 182613, 182614, 182615, 182616 were selected from the ORFs having the nucleotide sequence represented by SEQ ID NO: 173856.

The probes having the nucleotide sequence represented by SEQ ID NOS: 182617, 182618, 182619, 182620, 182621, 182622, 182623, 182624, 182625, 182626, 182627 were selected from the ORFs having the nucleotide sequence represented by SEQ ID NO: 173857.

The probes having the nucleotide sequence represented by SEQ ID NOS: 182628, 182629, 182630, 182631, 182632, 182633, 182634, 182635, 182636, 182637, 182638 were selected from the ORFs having the nucleotide sequence represented by SEQ ID NO: 173858.

The probes having the nucleotide sequence represented by SEQ ID NOS: 182639, 182640, 182641, 182642, 182643, 182644, 182645, 182646, 182647, 182648, 182649 were selected from the ORFs having the nucleotide sequence represented by SEQ ID NO: 173859.

The probes having the nucleotide sequence represented by SEQ ID NOS: 182650, 182651, 182652, 182653, 182654, 182655, 182656, 182657, 182658, 182659, 182660 were selected from the ORFs having the nucleotide sequence represented by SEQ ID NO: 173860.

The probes having the nucleotide sequence represented by SEQ ID NOS: 182661, 182662, 182663, 182664, 182665, 182666, 182667, 182668, 182669, 182670, 182671 were selected from the ORFs having the nucleotide sequence represented by SEQ ID NO: 173861.

The probes having the nucleotide sequence represented by SEQ ID NOS: 182672, 182673, 182674, 182675, 182676, 182677, 182678, 182679, 182680, 182681, 182682 were selected from the ORFs having the nucleotide sequence represented by SEQ ID NO: 173862.

The probes having the nucleotide sequence represented by SEQ ID NOS: 182683, 182684, 182685, 182686, 182687, 182688, 182689, 182690, 182691, 182692, 182693 were selected from the ORFs having the nucleotide sequence represented by SEQ ID NO: 173863.

The probes having the nucleotide sequence represented by SEQ ID NOS: 182694, 182695, 182696, 182697, 182698, 182699, 182700, 182701, 182702, 182703, 182704 were selected from the ORFs having the nucleotide sequence represented by SEQ ID NO: 173864.

The probes having the nucleotide sequence represented by SEQ ID NOS: 182705, 182706, 182707, 182708, 182709, 182710, 182711, 182712, 182713, 182714, 182715 were selected from the ORFs having the nucleotide sequence represented by SEQ ID NO: 173865.

The probes having the nucleotide sequence represented by SEQ ID NOS: 182716, 182717, 182718, 182719, 182720, 182721, 182722, 182723, 182724, 182725, 182726 were selected from the ORFs having the nucleotide sequence represented by SEQ ID NO: 173866.

The probes having the nucleotide sequence represented by SEQ ID NOS: 182727, 182728, 182729, 182730, 182731, 182732, 182733, 182734, 182735, 182736, 182737 were selected from the ORFs having the nucleotide sequence represented by SEQ ID NO: 173867.

The probes having the nucleotide sequence represented by SEQ ID NOS: 182738, 182739, 182740, 182741, 182742, 182743, 182744, 182745, 182746, 182747, 182748 were selected from the ORFs having the nucleotide sequence represented by SEQ ID NO: 173868.

The probes having the nucleotide sequence represented by SEQ ID NOS: 182749, 182750, 182751, 182752, 182753, 182754, 182755, 182756, 182757, 182758, 182759 were selected from the ORFs having the nucleotide sequence represented by SEQ ID NO: 173869.

The probes having the nucleotide sequence represented by SEQ ID NOS: 182760, 182761, 182762, 182763, 182764, 182765, 182766, 182767, 182768, 182769, 182770 were selected from the ORFs having the nucleotide sequence represented by SEQ ID NO: 173870.

The probes having the nucleotide sequence represented by SEQ ID NOS: 182771, 182772, 182773, 182774, 182775, 182776, 182777, 182778, 182779, 182780, 182781 were selected from the ORFs having the nucleotide sequence represented by SEQ ID NO: 173871.

The probes having the nucleotide sequence represented by SEQ ID NOS: 182782, 182783, 182784, 182785, 182786, 182787, 182788, 182789, 182790, 182791, 182792 were selected from the ORFs having the nucleotide sequence represented by SEQ ID NO: 173872.

The probes having the nucleotide sequence represented by SEQ ID NOS: 182793, 182794, 182795, 182796, 182797, 182798, 182799, 182800, 182801, 182802, 182803 were selected from the ORFs having the nucleotide sequence represented by SEQ ID NO: 173873.

The probes having the nucleotide sequence represented by SEQ ID NOS: 182804, 182805, 182806, 182807, 182808, 182809, 182810, 182811, 182812, 182813, 182814 were selected from the ORFs having the nucleotide sequence represented by SEQ ID NO: 173874.

The probes having the nucleotide sequence represented by SEQ ID NOS: 182815, 182816, 182817, 182818, 182819, 182820, 182821, 182822, 182823, 182824, 182825 were selected from the ORFs having the nucleotide sequence represented by SEQ ID NO: 173875.

The probes having the nucleotide sequence represented by SEQ ID NOS: 182826, 182827, 182828, 182829, 182830, 182831, 182832, 182833, 182834, 182835, 182836 were selected from the ORFs having the nucleotide sequence represented by SEQ ID NO: 173876.

The probes having the nucleotide sequence represented by SEQ ID NOS: 182837, 182838, 182839, 182840, 182841, 182842, 182843, 182844, 182845, 182846, 182847 were selected from the ORFs having the nucleotide sequence represented by SEQ ID NO: 173877.

The probes having the nucleotide sequence represented by SEQ ID NOS: 182848, 182849, 182850, 182851, 182852, 182853, 182854, 182855, 182856, 182857, 182858 were selected from the ORFs having the nucleotide sequence represented by SEQ ID NO: 173878.

The probes having the nucleotide sequence represented by SEQ ID NOS: 182859, 182860, 182861, 182862, 182863, 182864, 182865, 182866, 182867, 182868, 182869 were selected from the ORFs having the nucleotide sequence represented by SEQ ID NO: 173879.

The probes having the nucleotide sequence represented by SEQ ID NOS: 182870, 182871, 182872, 182873, 182874, 182875, 182876, 182877, 182878, 182879, 182880 were selected from the ORFs having the nucleotide sequence represented by SEQ ID NO: 173880.

The probes having the nucleotide sequence represented by SEQ ID NOS: 182881, 182882, 182883, 182884, 182885, 182886, 182887, 182888, 182889, 182890, 182891 were selected from the ORFs having the nucleotide sequence represented by SEQ ID NO: 173881.

The probes having the nucleotide sequence represented by SEQ ID NOS: 182892, 182893, 182894, 182895, 182896, 182897, 182898, 182899, 182900, 182901, 182902 were selected from the ORFs having the nucleotide sequence represented by SEQ ID NO: 173882.

The probes having the nucleotide sequence represented by SEQ ID NOS: 182903, 182904, 182905, 182906, 182907, 182908, 182909, 182910, 182911, 182912, 182913 were selected from the ORFs having the nucleotide sequence represented by SEQ ID NO: 173883.

The probes having the nucleotide sequence represented by SEQ ID NOS: 182914, 182915, 182916, 182917, 182918, 182919, 182920, 182921, 182922, 182923, 182924 were selected from the ORFs having the nucleotide sequence represented by SEQ ID NO: 173884.

The probes having the nucleotide sequence represented by SEQ ID NOS: 182925, 182926, 182927, 182928, 182929, 182930, 182931, 182932, 182933, 182934, 182935 were selected from the ORFs having the nucleotide sequence represented by SEQ ID NO: 173885.

The probes having the nucleotide sequence represented by SEQ ID NOS: 182936, 182937, 182938, 182939, 182940, 182941, 182942, 182943, 182944, 182945, 182946 were selected from the ORFs having the nucleotide sequence represented by SEQ ID NO: 173886.

The probes having the nucleotide sequence represented by SEQ ID NOS: 182947, 182948, 182949, 182950, 182951, 182952, 182953, 182954, 182955, 182956, 182957 were selected from the ORFs having the nucleotide sequence represented by SEQ ID NO: 173887.

The probes having the nucleotide sequence represented by SEQ ID NOS: 182958, 182959, 182960, 182961, 182962, 182963, 182964, 182965, 182966, 182967, 182968 were selected from the ORFs having the nucleotide sequence represented by SEQ ID NO: 173888.

The probes having the nucleotide sequence represented by SEQ ID NOS: 182969, 182970, 182971, 182972, 182973, 182974, 182975, 182976, 182977, 182978, 182979 were selected from the ORFs having the nucleotide sequence represented by SEQ ID NO: 173889.

The probes having the nucleotide sequence represented by SEQ ID NOS: 182980, 182981, 182982, 182983, 182984, 182985, 182986, 182987, 182988, 182989, 182990 were selected from the ORFs having the nucleotide sequence represented by SEQ ID NO: 173890.

The probes having the nucleotide sequence represented by SEQ ID NOS: 182991, 182992, 182993, 182994, 182995, 182996, 182997, 182998, 182999, 183000, 183001 were selected from the ORFs having the nucleotide sequence represented by SEQ ID NO: 173891.

The probes having the nucleotide sequence represented by SEQ ID NOS: 183002, 183003, 183004, 183005, 183006, 183007, 183008, 183009, 183010, 183011, 183012 were selected from the ORFs having the nucleotide sequence represented by SEQ ID NO: 173892.

The probes having the nucleotide sequence represented by SEQ ID NOS: 183013, 183014, 183015, 183016, 183017, 183018, 183019, 183020, 183021, 183022, 183023 were selected from the ORFs having the nucleotide sequence represented by SEQ ID NO: 173893.

The probes having the nucleotide sequence represented by SEQ ID NOS: 183024, 183025, 183026, 183027, 183028, 183029, 183030, 183031, 183032, 183033, 183034 were selected from the ORFs having the nucleotide sequence represented by SEQ ID NO: 173894.

The probes having the nucleotide sequence represented by SEQ ID NOS: 183035, 183036, 183037, 183038, 183039, 183040, 183041, 183042, 183043, 183044, 183045 were selected from the ORFs having the nucleotide sequence represented by SEQ ID NO: 173895.

The probes having the nucleotide sequence represented by SEQ ID NOS: 183046, 183047, 183048, 183049, 183050, 183051, 183052, 183053, 183054, 183055, 183056 were selected from the ORFs having the nucleotide sequence represented by SEQ ID NO: 173896.

The probes having the nucleotide sequence represented by SEQ ID NOS: 183057, 183058, 183059, 183060, 183061, 183062, 183063, 183064, 183065, 183066, 183067 were selected from the ORFs having the nucleotide sequence represented by SEQ ID NO: 173897.

The probes having the nucleotide sequence represented by SEQ ID NOS: 183068, 183069, 183070, 183071, 183072, 183073, 183074, 183075, 183076, 183077, 183078 were selected from the ORFs having the nucleotide sequence represented by SEQ ID NO: 173898.

The probes having the nucleotide sequence represented by SEQ ID NOS: 183079, 183080, 183081, 183082, 183083, 183084, 183085, 183086, 183087, 183088, 183089 were selected from the ORFs having the nucleotide sequence represented by SEQ ID NO: 173899.

The probes having the nucleotide sequence represented by SEQ ID NOS: 183090, 183091, 183092, 183093, 183094, 183095, 183096, 183097, 183098, 183099, 183100 were selected from the ORFs having the nucleotide sequence represented by SEQ ID NO: 173900.

The probes having the nucleotide sequence represented by SEQ ID NOS: 183101, 183102, 183103, 183104, 183105, 183106, 183107, 183108, 183109, 183110, 183111 were selected from the ORFs having the nucleotide sequence represented by SEQ ID NO: 173901.

The probes having the nucleotide sequence represented by SEQ ID NOS: 183112, 183113, 183114, 183115, 183116, 183117, 183118, 183119, 183120, 183121, 183122 were selected from the ORFs having the nucleotide sequence represented by SEQ ID NO: 173902.

The probes having the nucleotide sequence represented by SEQ ID NOS: 183123, 183124, 183125, 183126, 183127, 183128, 183129, 183130, 183131, 183132, 183133 were selected from the ORFs having the nucleotide sequence represented by SEQ ID NO: 173903.

The probes having the nucleotide sequence represented by SEQ ID NOS: 183134, 183135, 183136, 183137, 183138, 183139, 183140, 183141, 183142, 183143, 183144 were selected from the ORFs having the nucleotide sequence represented by SEQ ID NO: 173904.

The probes having the nucleotide sequence represented by SEQ ID NOS: 183145, 183146, 183147, 183148, 183149, 183150, 183151, 183152, 183153, 183154, 183155 were selected from the ORFs having the nucleotide sequence represented by SEQ ID NO: 173905.

The probes having the nucleotide sequence represented by SEQ ID NOS: 183156, 183157, 183158, 183159, 183160, 183161, 183162, 183163, 183164, 183165, 183166 were selected from the ORFs having the nucleotide sequence represented by SEQ ID NO: 173906.

The probes having the nucleotide sequence represented by SEQ ID NOS: 183167, 183168, 183169, 183170, 183171, 183172, 183173, 183174, 183175, 183176, 183177 were selected from the ORFs having the nucleotide sequence represented by SEQ ID NO: 173907.

The probes having the nucleotide sequence represented by SEQ ID NOS: 183178, 183179, 183180, 183181, 183182, 183183, 183184, 183185, 183186, 183187, 183188 were selected from the ORFs having the nucleotide sequence represented by SEQ ID NO: 173908.

The probes having the nucleotide sequence represented by SEQ ID NOS: 183189, 183190, 183191, 183192, 183193, 183194, 183195, 183196, 183197, 183198, 183199 were selected from the ORFs having the nucleotide sequence represented by SEQ ID NO: 173909.

The probes having the nucleotide sequence represented by SEQ ID NOS: 183200, 183201, 183202, 183203, 183204, 183205, 183206, 183207, 183208, 183209, 183210 were selected from the ORFs having the nucleotide sequence represented by SEQ ID NO: 173910.

The probes having the nucleotide sequence represented by SEQ ID NOS: 183211, 183212, 183213, 183214, 183215, 183216, 183217, 183218, 183219, 183220, 183221 were selected from the ORFs having the nucleotide sequence represented by SEQ ID NO: 173911.

The probes having the nucleotide sequence represented by SEQ ID NOS: 183222, 183223, 183224, 183225, 183226, 183227, 183228, 183229, 183230, 183231, 183232 were selected from the ORFs having the nucleotide sequence represented by SEQ ID NO: 173912.

The probes having the nucleotide sequence represented by SEQ ID NOS: 183233, 183234, 183235, 183236, 183237, 183238, 183239, 183240, 183241, 183242, 183243 were selected from the ORFs having the nucleotide sequence represented by SEQ ID NO: 173913.

The probes having the nucleotide sequence represented by SEQ ID NOS: 183244, 183245, 183246, 183247, 183248, 183249, 183250, 183251, 183252, 183253, 183254 were selected from the ORFs having the nucleotide sequence represented by SEQ ID NO: 173914.

The probes having the nucleotide sequence represented by SEQ ID NOS: 183255, 183256, 183257, 183258, 183259, 183260, 183261, 183262, 183263, 183264, 183265 were selected from the ORFs having the nucleotide sequence represented by SEQ ID NO: 173915.

The probes having the nucleotide sequence represented by SEQ ID NOS: 183266, 183267, 183268, 183269, 183270, 183271, 183272, 183273, 183274, 183275, 183276 were selected from the ORFs having the nucleotide sequence represented by SEQ ID NO: 173916.

The probes having the nucleotide sequence represented by SEQ ID NOS: 183277, 183278, 183279, 183280, 183281, 183282, 183283, 183284, 183285, 183286, 183287 were selected from the ORFs having the nucleotide sequence represented by SEQ ID NO: 173917.

The probes having the nucleotide sequence represented by SEQ ID NOS: 183288, 183289, 183290, 183291, 183292, 183293, 183294, 183295, 183296, 183297, 183298 were selected from the ORFs having the nucleotide sequence represented by SEQ ID NO: 173918.

The probes having the nucleotide sequence represented by SEQ ID NOS: 183299, 183300, 183301, 183302, 183303, 183304, 183305, 183306, 183307, 183308, 183309 were selected from the ORFs having the nucleotide sequence represented by SEQ ID NO: 173919.

The probes having the nucleotide sequence represented by SEQ ID NOS: 183310, 183311, 183312, 183313, 183314, 183315, 183316, 183317, 183318, 183319, 183320 were selected from the ORFs having the nucleotide sequence represented by SEQ ID NO: 173920.

The probes having the nucleotide sequence represented by SEQ ID NOS: 183321, 183322, 183323, 183324, 183325, 183326, 183327, 183328, 183329, 183330, 183331 were selected from the ORFs having the nucleotide sequence represented by SEQ ID NO: 173921.

The probes having the nucleotide sequence represented by SEQ ID NOS: 183332, 183333, 183334, 183335, 183336, 183337, 183338, 183339, 183340, 183341, 183342 were selected from the ORFs having the nucleotide sequence represented by SEQ ID NO: 173922.

The probes having the nucleotide sequence represented by SEQ ID NOS: 183343, 183344, 183345, 183346, 183347, 183348, 183349, 183350, 183351, 183352, 183353 were selected from the ORFs having the nucleotide sequence represented by SEQ ID NO: 173923.

The probes having the nucleotide sequence represented by SEQ ID NOS: 183354, 183355, 183356, 183357, 183358, 183359, 183360, 183361, 183362, 183363, 183364 were selected from the ORFs having the nucleotide sequence represented by SEQ ID NO: 173924.

The probes having the nucleotide sequence represented by SEQ ID NOS: 183365, 183366, 183367, 183368, 183369, 183370, 183371, 183372, 183373, 183374, 183375 were selected from the ORFs having the nucleotide sequence represented by SEQ ID NO: 173925.

The probes having the nucleotide sequence represented by SEQ ID NOS: 183376, 183377, 183378, 183379, 183380, 183381, 183382, 183383, 183384, 183385, 183386 were selected from the ORFs having the nucleotide sequence represented by SEQ ID NO: 173926.

The probes having the nucleotide sequence represented by SEQ ID NOS: 183387, 183388, 183389, 183390, 183391, 183392, 183393, 183394, 183395, 183396, 183397 were selected from the ORFs having the nucleotide sequence represented by SEQ ID NO: 173927.

The probes having the nucleotide sequence represented by SEQ ID NOS: 183398, 183399, 183400, 183401, 183402, 183403, 183404, 183405, 183406, 183407, 183408 were selected from the ORFs having the nucleotide sequence represented by SEQ ID NO: 173928.

The probes having the nucleotide sequence represented by SEQ ID NOS: 183409, 183410, 183411, 183412, 183413, 183414, 183415, 183416, 183417, 183418, 183419 were selected from the ORFs having the nucleotide sequence represented by SEQ ID NO: 173929.

The probes having the nucleotide sequence represented by SEQ ID NOS: 183420, 183421, 183422, 183423, 183424, 183425, 183426, 183427, 183428, 183429, 183430 were selected from the ORFs having the nucleotide sequence represented by SEQ ID NO: 173930.

The probes having the nucleotide sequence represented by SEQ ID NOS: 183431, 183432, 183433, 183434, 183435, 183436, 183437, 183438, 183439, 183440, 183441 were selected from the ORFs having the nucleotide sequence represented by SEQ ID NO: 173931.

The probes having the nucleotide sequence represented by SEQ ID NOS: 183442, 183443, 183444, 183445, 183446, 183447, 183448, 183449, 183450, 183451, 183452 were selected from the ORFs having the nucleotide sequence represented by SEQ ID NO: 173932.

The probes having the nucleotide sequence represented by SEQ ID NOS: 183453, 183454, 183455, 183456, 183457, 183458, 183459, 183460 were selected from the ORFs having the nucleotide sequence represented by SEQ ID NO: 173933.

The probes having the nucleotide sequence represented by SEQ ID NOS: 183461, 183462, 183463, 183464, 183465, 183466, 183467, 183468, 183469, 183470, 183471 were selected from the ORFs having the nucleotide sequence represented by SEQ ID NO: 173934.

The probes having the nucleotide sequence represented by SEQ ID NOS: 183472, 183473, 183474, 183475, 183476, 183477, 183478, 183479, 183480, 183481, 183482 were selected from the ORFs having the nucleotide sequence represented by SEQ ID NO: 173935.

The probes having the nucleotide sequence represented by SEQ ID NOS: 183483, 183484, 183485, 183486, 183487, 183488, 183489, 183490, 183491, 183492, 183493 were selected from the ORFs having the nucleotide sequence represented by SEQ ID NO: 173936.

The probes having the nucleotide sequence represented by SEQ ID NOS: 183494, 183495, 183496, 183497, 183498, 183499, 183500, 183501, 183502, 183503, 183504 were selected from the ORFs having the nucleotide sequence represented by SEQ ID NO: 173937.

The probes having the nucleotide sequence represented by SEQ ID NOS: 183505, 183506, 183507, 183508, 183509, 183510, 183511, 183512, 183513, 183514, 183515 were selected from the ORFs having the nucleotide sequence represented by SEQ ID NO: 173938.

The probes having the nucleotide sequence represented by SEQ ID NOS: 183516, 183517, 183518, 183519, 183520, 183521, 183522, 183523, 183524, 183525, 183526 were selected from the ORFs having the nucleotide sequence represented by SEQ ID NO: 173939.

The probes having the nucleotide sequence represented by SEQ ID NOS: 183527, 183528, 183529, 183530, 183531, 183532, 183533, 183534, 183535, 183536, 183537 were selected from the ORFs having the nucleotide sequence represented by SEQ ID NO: 173940.

The probes having the nucleotide sequence represented by SEQ ID NOS: 183538, 183539, 183540, 183541, 183542, 183543, 183544, 183545, 183546, 183547, 183548 were selected from the ORFs having the nucleotide sequence represented by SEQ ID NO: 173941.

The probes having the nucleotide sequence represented by SEQ ID NOS: 183549, 183550, 183551, 183552, 183553, 183554, 183555, 183556, 183557, 183558, 183559 were selected from the ORFs having the nucleotide sequence represented by SEQ ID NO: 173942.

The probes having the nucleotide sequence represented by SEQ ID NOS: 183560, 183561, 183562, 183563, 183564, 183565, 183566, 183567, 183568, 183569, 183570 were selected from the ORFs having the nucleotide sequence represented by SEQ ID NO: 173943.

The probes having the nucleotide sequence represented by SEQ ID NOS: 183571, 183572, 183573, 183574, 183575, 183576, 183577, 183578, 183579, 183580, 183581 were selected from the ORFs having the nucleotide sequence represented by SEQ ID NO: 173944.

The probes having the nucleotide sequence represented by SEQ ID NOS: 183582, 183583, 183584, 183585, 183586, 183587, 183588, 183589, 183590, 183591, 183592 were selected from the ORFs having the nucleotide sequence represented by SEQ ID NO: 173945.

The probes having the nucleotide sequence represented by SEQ ID NOS: 183593, 183594, 183595, 183596, 183597, 183598, 183599, 183600, 183601, 183602, 183603 were selected from the ORFs having the nucleotide sequence represented by SEQ ID NO: 173946.

The probes having the nucleotide sequence represented by SEQ ID NOS: 183604, 183605, 183606, 183607, 183608, 183609, 183610, 183611, 183612, 183613, 183614 were selected from the ORFs having the nucleotide sequence represented by SEQ ID NO: 173947.

The probes having the nucleotide sequence represented by SEQ ID NOS: 183615, 183616, 183617, 183618, 183619, 183620, 183621, 183622, 183623, 183624, 183625 were selected from the ORFs having the nucleotide sequence represented by SEQ ID NO: 173948.

The probes having the nucleotide sequence represented by SEQ ID NOS: 183626, 183627, 183628, 183629, 183630, 183631, 183632, 183633, 183634, 183635, 183636 were selected from the ORFs having the nucleotide sequence represented by SEQ ID NO: 173949.

The probes having the nucleotide sequence represented by SEQ ID NOS: 183637, 183638, 183639, 183640, 183641, 183642, 183643, 183644, 183645, 183646, 183647 were selected from the ORFs having the nucleotide sequence represented by SEQ ID NO: 173950.

The probes having the nucleotide sequence represented by SEQ ID NOS: 183648, 183649, 183650, 183651, 183652, 183653, 183654, 183655, 183656, 183657, 183658 were selected from the ORFs having the nucleotide sequence represented by SEQ ID NO: 173951.

The probes having the nucleotide sequence represented by SEQ ID NOS: 183659, 183660, 183661, 183662, 183663, 183664, 183665, 183666, 183667, 183668, 183669 were selected from the ORFs having the nucleotide sequence represented by SEQ ID NO: 173952.

The probes having the nucleotide sequence represented by SEQ ID NOS: 183670, 183671, 183672, 183673, 183674, 183675, 183676, 183677, 183678, 183679, 183680 were selected from the ORFs having the nucleotide sequence represented by SEQ ID NO: 173953.

The probes having the nucleotide sequence represented by SEQ ID NOS: 183681, 183682, 183683, 183684, 183685, 183686, 183687, 183688, 183689, 183690, 183691 were selected from the ORFs having the nucleotide sequence represented by SEQ ID NO: 173954.

The probes having the nucleotide sequence represented by SEQ ID NOS: 183692, 183693, 183694, 183695, 183696, 183697, 183698, 183699, 183700, 183701, 183702 were selected from the ORFs having the nucleotide sequence represented by SEQ ID NO: 173955.

The probes having the nucleotide sequence represented by SEQ ID NOS: 183703, 183704, 183705, 183706, 183707, 183708, 183709, 183710, 183711, 183712, 183713 were selected from the ORFs having the nucleotide sequence represented by SEQ ID NO: 173956.

The probes having the nucleotide sequence represented by SEQ ID NOS: 183714, 183715, 183716, 183717, 183718, 183719, 183720, 183721, 183722, 183723, 183724 were selected from the ORFs having the nucleotide sequence represented by SEQ ID NO: 173957.

The probes having the nucleotide sequence represented by SEQ ID NOS: 183725, 183726, 183727, 183728, 183729, 183730, 183731, 183732, 183733, 183734, 183735 were selected from the ORFs having the nucleotide sequence represented by SEQ ID NO: 173958.

The probes having the nucleotide sequence represented by SEQ ID NOS: 183736, 183737, 183738, 183739, 183740, 183741, 183742, 183743, 183744, 183745, 183746 were selected from the ORFs having the nucleotide sequence represented by SEQ ID NO: 173959.

The probes having the nucleotide sequence represented by SEQ ID NOS: 183747, 183748, 183749, 183750, 183751, 183752, 183753, 183754, 183755, 183756, 183757 were selected from the ORFs having the nucleotide sequence represented by SEQ ID NO: 173960.

The probes having the nucleotide sequence represented by SEQ ID NOS: 183758, 183759, 183760, 183761, 183762, 183763, 183764, 183765, 183766, 183767, 183768 were selected from the ORFs having the nucleotide sequence represented by SEQ ID NO: 173961.

The probes having the nucleotide sequence represented by SEQ ID NOS: 183769, 183770, 183771, 183772, 183773, 183774, 183775, 183776, 183777, 183778, 183779 were selected from the ORFs having the nucleotide sequence represented by SEQ ID NO: 173962.

The probes having the nucleotide sequence represented by SEQ ID NOS: 183780, 183781, 183782, 183783, 183784, 183785, 183786, 183787, 183788, 183789, 183790 were selected from the ORFs having the nucleotide sequence represented by SEQ ID NO: 173963.

The probes having the nucleotide sequence represented by SEQ ID NOS: 183791, 183792, 183793, 183794, 183795, 183796, 183797, 183798, 183799, 183800, 183801 were selected from the ORFs having the nucleotide sequence represented by SEQ ID NO: 173964.

The probes having the nucleotide sequence represented by SEQ ID NOS: 183802, 183803, 183804, 183805, 183806, 183807, 183808, 183809, 183810, 183811, 183812 were selected from the ORFs having the nucleotide sequence represented by SEQ ID NO: 173965.

The probes having the nucleotide sequence represented by SEQ ID NOS: 183813, 183814, 183815, 183816, 183817, 183818, 183819, 183820, 183821, 183822, 183823 were selected from the ORFs having the nucleotide sequence represented by SEQ ID NO: 173966.

The probes having the nucleotide sequence represented by SEQ ID NOS: 183824, 183825, 183826, 183827, 183828, 183829, 183830, 183831, 183832, 183833, 183834 were selected from the ORFs having the nucleotide sequence represented by SEQ ID NO: 173967.

The probes having the nucleotide sequence represented by SEQ ID NOS: 183835, 183836, 183837, 183838, 183839, 183840, 183841, 183842, 183843, 183844, 183845 were selected from the ORFs having the nucleotide sequence represented by SEQ ID NO: 173968.

The probes having the nucleotide sequence represented by SEQ ID NOS: 183846, 183847, 183848,. 183849, 183850, 183851, 183852, 183853, 183854, 183855, 183856 were selected from the ORFs having the nucleotide sequence represented by SEQ ID NO: 173969.

The probes having the nucleotide sequence represented by SEQ ID NOS: 183857, 183858, 183859, 183860, 183861, 183862, 183863, 183864, 183865, 183866, 183867 were selected from the ORFs having the nucleotide sequence represented by SEQ ID NO: 173970.

The probes having the nucleotide sequence represented by SEQ ID NOS: 183868, 183869, 183870, 183871, 183872, 183873, 183874, 183875, 183876, 183877, 183878 were selected from the ORFs having the nucleotide sequence represented by SEQ ID NO: 173971.

The probes having the nucleotide sequence represented by SEQ ID NOS: 183879, 183880, 183881, 183882, 183883, 183884, 183885, 183886, 183887, 183888, 183889 were selected from the ORFs having the nucleotide sequence represented by SEQ ID NO: 173972.

The probes having the nucleotide sequence represented by SEQ ID NOS: 183890, 183891, 183892, 183893, 183894, 183895, 183896, 183897, 183898, 183899, 183900 were selected from the ORFs having the nucleotide sequence represented by SEQ ID NO: 173973.

The probes having the nucleotide sequence represented by SEQ ID NOS: 183901, 183902, 183903, 183904, 183905, 183906, 183907, 183908, 183909, 183910, 183911 were selected from the ORFs having the nucleotide sequence represented by SEQ ID NO: 173974.

The probes having the nucleotide sequence represented by SEQ ID NOS: 183912, 183913, 183914, 183915, 183916, 183917, 183918, 183919, 183920, 183921, 183922 were selected from the ORFs having the nucleotide sequence represented by SEQ ID NO: 173975.

The probes having the nucleotide sequence represented by SEQ ID NOS: 183923, 183924, 183925, 183926, 183927, 183928, 183929, 183930, 183931, 183932, 183933 were selected from the ORFs having the nucleotide sequence represented by SEQ ID NO: 173976.

The probes having the nucleotide sequence represented by SEQ ID NOS: 183934, 183935, 183936, 183937, 183938, 183939, 183940, 183941, 183942, 183943, 183944 were selected from the ORFs having the nucleotide sequence represented by SEQ ID NO: 173977.

The probes having the nucleotide sequence represented by SEQ ID NOS: 183945, 183946, 183947, 183948, 183949, 183950, 183951, 183952, 183953, 183954, 183955 were selected from the ORFs having the nucleotide sequence represented by SEQ ID NO: 173978.

The probes having the nucleotide sequence represented by SEQ ID NOS: 183956, 183957, 183958, 183959, 183960, 183961, 183962, 183963, 183964, 183965, 183966 were selected from the ORFs having the nucleotide sequence represented by SEQ ID NO: 173979.

The probes having the nucleotide sequence represented by SEQ ID NOS: 183967, 183968, 183969, 183970, 183971, 183972, 183973, 183974, 183975, 183976, 183977 were selected from the ORFs having the nucleotide sequence represented by SEQ ID NO: 173980.

The probes having the nucleotide sequence represented by SEQ ID NOS: 183978, 183979, 183980, 183981, 183982, 183983, 183984, 183985, 183986, 183987, 183988 were selected from the ORFs having the nucleotide sequence represented by SEQ ID NO: 173981.

The probes having the nucleotide sequence represented by SEQ ID NOS: 183989, 183990, 183991, 183992, 183993, 183994, 183995, 183996, 183997, 183998, 183999 were selected from the ORFs having the nucleotide sequence represented by SEQ ID NO: 173982.

The probes having the nucleotide sequence represented by SEQ ID NOS: 184000, 184001, 184002, 184003, 184004, 184005, 184006, 184007, 184008, 184009, 184010 were selected from the ORFs having the nucleotide sequence represented by SEQ ID NO: 173983.

The probes having the nucleotide sequence represented by SEQ ID NOS: 184011, 184012, 184013, 184014, 184015, 184016, 184017, 184018, 184019, 184020, 184021 were selected from the ORFs having the nucleotide sequence represented by SEQ ID NO: 173984.

The probes having the nucleotide sequence represented by SEQ ID NOS: 184022, 184023, 184024, 184025, 184026, 184027, 184028, 184029, 184030, 184031, 184032 were selected from the ORFs having the nucleotide sequence represented by SEQ ID NO: 173985.

The probes having the nucleotide sequence represented by SEQ ID NOS: 184033, 184034, 184035, 184036, 184037, 184038, 184039, 184040, 184041, 184042, 184043 were selected from the ORFs having the nucleotide sequence represented by SEQ ID NO: 173986.

The probes having the nucleotide sequence represented by SEQ ID NOS: 184044, 184045, 184046, 184047, 184048, 184049, 184050, 184051, 184052, 184053, 184054 were selected from the ORFs having the nucleotide sequence represented by SEQ ID NO: 173987.

The probes having the nucleotide sequence represented by SEQ ID NOS: 184055, 184056, 184057, 184058, 184059, 184060, 184061, 184062, 184063, 184064, 184065 were selected from the ORFs having the nucleotide sequence represented by SEQ ID NO: 173988.

The probes having the nucleotide sequence represented by SEQ ID NOS: 184066, 184067, 184068, 184069, 184070, 184071, 184072, 184073, 184074, 184075, 184076 were selected from the ORFs having the nucleotide sequence represented by SEQ ID NO: 173989.

The probes having the nucleotide sequence represented by SEQ ID NOS: 184077, 184078, 184079, 184080, 184081, 184082, 184083, 184084, 184085, 184086, 184087 were selected from the ORFs having the nucleotide sequence represented by SEQ ID NO: 173990.

The probes having the nucleotide sequence represented by SEQ ID NOS: 184088, 184089, 184090, 184091, 184092, 184093, 184094, 184095, 184096, 184097, 184098 were selected from the ORFs having the nucleotide sequence represented by SEQ ID NO: 173991.

The probes having the nucleotide sequence represented by SEQ ID NOS: 184099, 184100, 184101, 184102, 184103, 184104, 184105, 184106, 184107, 184108, 184109 were selected from the ORFs having the nucleotide sequence represented by SEQ ID NO: 173992.

The probes having the nucleotide sequence represented by SEQ ID NOS: 184110, 184111, 184112, 184113, 184114, 184115, 184116, 184117, 184118, 184119, 184120 were selected from the ORFs having the nucleotide sequence represented by SEQ ID NO: 173993.

The probes having the nucleotide sequence represented by SEQ ID NOS: 184121, 184122, 184123, 184124, 184125, 184126, 184127, 184128, 184129, 184130, 184131 were selected from the ORFs having the nucleotide sequence represented by SEQ ID NO: 173994.

The probes having the nucleotide sequence represented by SEQ ID NOS: 184132, 184133, 184134, 184135, 184136, 184137, 184138, 184139, 184140, 184141, 184142 were selected from the ORFs having the nucleotide sequence represented by SEQ ID NO: 173995.

The probes having the nucleotide sequence represented by SEQ ID NOS: 184143, 184144, 184145, 184146, 184147, 184148, 184149, 184150, 184151, 184152, 184153 were selected from the ORFs having the nucleotide sequence represented by SEQ ID NO: 173996.

The probes having the nucleotide sequence represented by SEQ ID NOS: 184154, 184155, 184156, 184157, 184158, 184159, 184160, 184161, 184162, 184163, 184164 were selected from the ORFs having the nucleotide sequence represented by SEQ ID NO: 173997.

The probes having the nucleotide sequence represented by SEQ ID NOS: 184165, 184166, 184167, 184168, 184169, 184170, 184171, 184172, 184173, 184174, 184175 were selected from the ORFs having the nucleotide sequence represented by SEQ ID NO: 173998.

The probes having the nucleotide sequence represented by SEQ ID NOS: 184176, 184177, 184178, 184179, 184180, 184181, 184182, 184183, 184184, 184185, 184186 were selected from the ORFs having the nucleotide sequence represented by SEQ ID NO: 173999.

The probes having the nucleotide sequence represented by SEQ ID NOS: 184187, 184188, 184189, 184190, 184191, 184192, 184193, 184194, 184195, 184196, 184197 were selected from the ORFs having the nucleotide sequence represented by SEQ ID NO: 174000.

The probes having the nucleotide sequence represented by SEQ ID NOS: 184198, 184199, 184200, 184201, 184202, 184203, 184204, 184205, 184206, 184207, 184208 were selected from the ORFs having the nucleotide sequence represented by SEQ ID NO: 174001.

The probes having the nucleotide sequence represented by SEQ ID NOS: 184209, 184210, 184211, 184212, 184213, 184214, 184215, 184216, 184217, 184218, 184219 were selected from the ORFs having the nucleotide sequence represented by SEQ ID NO: 174002.

The probes having the nucleotide sequence represented by SEQ ID NOS: 184220, 184221, 184222, 184223, 184224, 184225, 184226, 184227, 184228, 184229, 184230 were selected from the ORFs having the nucleotide sequence represented by SEQ ID NO: 174003.

The probes having the nucleotide sequence represented by SEQ ID NOS: 184231, 184232, 184233, 184234, 184235, 184236, 184237, 184238, 184239, 184240, 184241 were selected from the ORFs having the nucleotide sequence represented by SEQ ID NO: 174004.

The probes having the nucleotide sequence represented by SEQ ID NOS: 184242, 184243, 184244, 184245, 184246, 184247, 184248, 184249, 184250, 184251, 184252 were selected from the ORFs having the nucleotide sequence represented by SEQ ID NO: 174005.

The probes having the nucleotide sequence represented by SEQ ID NOS: 184253, 184254, 184255, 184256, 184257, 184258, 184259, 184260, 184261, 184262, 184263 were selected from the ORFs having the nucleotide sequence represented by SEQ ID NO: 174006.

The probes having the nucleotide sequence represented by SEQ ID NOS: 184264, 184265, 184266, 184267, 184268, 184269, 184270, 184271, 184272, 184273, 184274 were selected from the ORFs having the nucleotide sequence represented by SEQ ID NO: 174007.

The probes having the nucleotide sequence represented by SEQ ID NOS: 184275, 184276, 184277, 184278, 184279, 184280, 184281, 184282, 184283, 184284, 184285 were selected from the ORFs having the nucleotide sequence represented by SEQ ID NO: 174008.

The probes having the nucleotide sequence represented by SEQ ID NOS: 184286, 184287, 184288, 184289, 184290, 184291, 184292, 184293, 184294, 184295, 184296 were selected from the ORFs having the nucleotide sequence represented by SEQ ID NO: 174009.

The probes having the nucleotide sequence represented by SEQ ID NOS: 184297, 184298, 184299, 184300, 184301, 184302, 184303, 184304, 184305, 184306, 184307 were selected from the ORFs having the nucleotide sequence represented by SEQ ID NO: 174010.

The probes having the nucleotide sequence represented by SEQ ID NOS: 184308, 184309, 184310, 184311, 184312, 184313, 184314, 184315, 184316, 184317, 184318 were selected from the ORFs having the nucleotide sequence represented by SEQ ID NO: 174011.

The probes having the nucleotide sequence represented by SEQ ID NOS: 184319, 184320, 184321, 184322, 184323, 184324, 184325, 184326, 184327, 184328, 184329 were selected from the ORFs having the nucleotide sequence represented by SEQ ID NO: 174012.

The probes having the nucleotide sequence represented by SEQ ID NOS: 184330, 184331, 184332, 184333, 184334, 184335, 184336, 184337, 184338, 184339, 184340 were selected from the ORFs having the nucleotide sequence represented by SEQ ID NO: 174013.

The probes having the nucleotide sequence represented by SEQ ID NOS: 184341, 184342, 184343, 184344, 184345, 184346, 184347, 184348, 184349, 184350, 184351 were selected from the ORFs having the nucleotide sequence represented by SEQ ID NO: 174014.

The probes having the nucleotide sequence represented by SEQ ID NOS: 184352, 184353, 184354, 184355, 184356, 184357, 184358, 184359, 184360, 184361, 184362 were selected from the ORFs having the nucleotide sequence represented by SEQ ID NO: 174015.

The probes having the nucleotide sequence represented by SEQ ID NOS: 184363, 184364, 184365, 184366, 184367, 184368, 184369, 184370, 184371, 184372, 184373 were selected from the ORFs having the nucleotide sequence represented by SEQ ID NO: 174016.

The probes having the nucleotide sequence represented by SEQ ID NOS: 184374, 184375, 184376, 184377, 184378, 184379, 184380, 184381, 184382, 184383, 184384 were selected from the ORFs having the nucleotide sequence represented by SEQ ID NO: 174017.

The probes having the nucleotide sequence represented by SEQ ID NOS: 184385, 184386, 184387, 184388, 184389, 184390, 184391, 184392, 184393, 184394, 184395 were selected from the ORFs having the nucleotide sequence represented by SEQ ID NO: 174018.

The probes having the nucleotide sequence represented by SEQ ID NOS: 184396, 184397, 184398, 184399, 184400, 184401, 184402, 184403, 184404, 184405, 184406 were selected from the ORFs having the nucleotide sequence represented by SEQ ID NO: 174019.

The probes having the nucleotide sequence represented by SEQ ID NOS: 184407, 184408, 184409, 184410, 184411, 184412, 184413, 184414, 184415, 184416, 184417 were selected from the ORFs having the nucleotide sequence represented by SEQ ID NO: 174020.

The probes having the nucleotide sequence represented by SEQ ID NOS: 184418, 184419, 184420, 184421, 184422, 184423, 184424, 184425, 184426, 184427, 184428 were selected from the ORFs having the nucleotide sequence represented by SEQ ID NO: 174021.

The probes having the nucleotide sequence represented by SEQ ID NOS: 184429, 184430, 184431, 184432, 184433, 184434, 184435, 184436, 184437, 184438, 184439 were selected from the ORFs having the nucleotide sequence represented by SEQ ID NO: 174022.

The probes having the nucleotide sequence represented by SEQ ID NOS: 184440, 184441, 184442, 184443, 184444, 184445, 184446, 184447, 184448, 184449, 184450 were selected from the ORFs having the nucleotide sequence represented by SEQ ID NO: 174023.

The probes having the nucleotide sequence represented by SEQ ID NOS: 184451, 184452, 184453, 184454, 184455, 184456, 184457, 184458, 184459, 184460, 184461 were selected from the ORFs having the nucleotide sequence represented by SEQ ID NO: 174024.

The probes having the nucleotide sequence represented by SEQ ID NOS: 184462, 184463, 184464, 184465, 184466, 184467, 184468, 184469, 184470, 184471, 184472 were selected from the ORFs having the nucleotide sequence represented by SEQ ID NO: 174025.

The probes having the nucleotide sequence represented by SEQ ID NOS: 184473, 184474, 184475, 184476, 184477, 184478, 184479, 184480, 184481, 184482, 184483 were selected from the ORFs having the nucleotide sequence represented by SEQ ID NO: 174026.

The probes having the nucleotide sequence represented by SEQ ID NOS: 184484, 184485, 184486, 184487, 184488, 184489, 184490, 184491, 184492, 184493, 184494 were selected from the ORFs having the nucleotide sequence represented by SEQ ID NO: 174027.

The probes having the nucleotide sequence represented by SEQ ID NOS: 184495, 184496, 184497, 184498, 184499, 184500, 184501, 184502, 184503, 184504, 184505 were selected from the ORFs having the nucleotide sequence represented by SEQ ID NO: 174028.

The probes having the nucleotide sequence represented by SEQ ID NOS: 184506, 184507, 184508, 184509, 184510, 184511, 184512, 184513, 184514, 184515, 184516 were selected from the ORFs having the nucleotide sequence represented by SEQ ID NO: 174029.

The probes having the nucleotide sequence represented by SEQ ID NOS: 184517, 184518, 184519, 184520, 184521, 184522, 184523, 184524, 184525, 184526, 184527 were selected from the ORFs having the nucleotide sequence represented by SEQ ID NO: 174030.

The probes having the nucleotide sequence represented by SEQ ID NOS: 184528, 184529, 184530, 184531, 184532, 184533, 184534, 184535, 184536, 184537, 184538 were selected from the ORFs having the nucleotide sequence represented by SEQ ID NO: 174031.

The probes having the nucleotide sequence represented by SEQ ID NOS: 184539, 184540, 184541, 184542, 184543, 184544, 184545, 184546, 184547, 184548, 184549 were selected from the ORFs having the nucleotide sequence represented by SEQ ID NO: 174032.

The probes having the nucleotide sequence represented by SEQ ID NOS: 184550, 184551, 184552, 184553, 184554, 184555, 184556, 184557, 184558, 184559, 184560 were selected from the ORFs having the nucleotide sequence represented by SEQ ID NO: 174033.

The probes having the nucleotide sequence represented by SEQ ID NOS: 184561, 184562, 184563, 184564, 184565, 184566, 184567, 184568, 184569, 184570, 184571 were selected from the ORFs having the nucleotide sequence represented by SEQ ID NO: 174034.

The probes having the nucleotide sequence represented by SEQ ID NOS: 184572, 184573, 184574, 184575, 184576, 184577, 184578, 184579, 184580, 184581, 184582 were selected from the ORFs having the nucleotide sequence represented by SEQ ID NO: 174035.

The probes having the nucleotide sequence represented by SEQ ID NOS: 184583, 184584, 184585, 184586, 184587, 184588, 184589, 184590, 184591, 184592, 184593 were selected from the ORFs having the nucleotide sequence represented by SEQ ID NO: 174036.

The probes having the nucleotide sequence represented by SEQ ID NOS: 184594, 184595, 184596, 184597, 184598, 184599, 184600, 184601, 184602, 184603, 184604 were selected from the ORFs having the nucleotide sequence represented by SEQ ID NO: 174037.

The probes having the nucleotide sequence represented by SEQ ID NOS: 184605, 184606, 184607, 184608, 184609, 184610, 184611, 184612, 184613, 184614, 184615 were selected from the ORFs having the nucleotide sequence represented by SEQ ID NO: 174038.

The probes having the nucleotide sequence represented by SEQ ID NOS: 184616, 184617, 184618, 184619, 184620, 184621, 184622, 184623, 184624, 184625, 184626 were selected from the ORFs having the nucleotide sequence represented by SEQ ID NO: 174039.

The probes having the nucleotide sequence represented by SEQ ID NOS: 184627, 184628, 184629, 184630, 184631, 184632, 184633, 184634, 184635, 184636, 184637 were selected from the ORFs having the nucleotide sequence represented by SEQ ID NO: 174040.

The probes having the nucleotide sequence represented by SEQ ID NOS: 184638, 184639, 184640, 184641, 184642, 184643, 184644, 184645, 184646, 184647, 184648 were selected from the ORFs having the nucleotide sequence represented by SEQ ID NO: 174041.

The probes having the nucleotide sequence represented by SEQ ID NOS: 184649, 184650, 184651, 184652, 184653, 184654, 184655, 184656, 184657, 184658, 184659 were selected from the ORFs having the nucleotide sequence represented by SEQ ID NO: 174042.

The probes having the nucleotide sequence represented by SEQ ID NOS: 184660, 184661, 184662, 184663, 184664, 184665, 184666, 184667, 184668, 184669, 184670 were selected from the ORFs having the nucleotide sequence represented by SEQ ID NO: 174043.

The probes having the nucleotide sequence represented by SEQ ID NOS: 184671, 184672, 184673, 184674, 184675, 184676, 184677, 184678, 184679, 184680, 184681 were selected from the ORFs having the nucleotide sequence represented by SEQ ID NO: 174044.

The probes having the nucleotide sequence represented by SEQ ID NOS: 184682, 184683, 184684, 184685, 184686, 184687, 184688, 184689, 184690, 184691, 184692 were selected from the ORFs having the nucleotide sequence represented by SEQ ID NO: 174045.

The probes having the nucleotide sequence represented by SEQ ID NOS: 184693, 184694, 184695, 184696, 184697, 184698, 184699, 184700, 184701, 184702, 184703 were selected from the ORFs having the nucleotide sequence represented by SEQ ID NO: 174046.

The probes having the nucleotide sequence represented by SEQ ID NOS: 184704, 184705, 184706, 184707, 184708, 184709, 184710, 184711, 184712 were selected from the ORFs having the nucleotide sequence represented by SEQ ID NO: 174047.

The probes having the nucleotide sequence represented by SEQ ID NOS: 184713, 184714, 184715, 184716, 184717, 184718, 184719, 184720, 184721, 184722, 184723 were selected from the ORFs having the nucleotide sequence represented by SEQ ID NO: 174048.

The probes having the nucleotide sequence represented by SEQ ID NOS: 184724, 184725, 184726, 184727, 184728, 184729, 184730, 184731, 184732, 184733, 184734 were selected from the ORFs having the nucleotide sequence represented by SEQ ID NO: 174049.

The probes having the nucleotide sequence represented by SEQ ID NOS: 184735, 184736, 184737, 184738, 184739, 184740, 184741, 184742, 184743, 184744, 184745 were selected from the ORFs having the nucleotide sequence represented by SEQ ID NO: 174050.

The probes having the nucleotide sequence represented by SEQ ID NOS: 184746, 184747, 184748, 184749, 184750, 184751, 184752, 184753, 184754, 184755, 184756 were selected from the ORFs having the nucleotide sequence represented by SEQ ID NO: 174051.

The probes having the nucleotide sequence represented by SEQ ID NOS: 184757, 184758, 184759, 184760, 184761, 184762, 184763, 184764, 184765, 184766, 184767 were selected from the ORFs having the nucleotide sequence represented by SEQ ID NO: 174052.

The probes having the nucleotide sequence represented by SEQ ID NOS: 184768, 184769, 184770, 184771, 184772, 184773, 184774, 184775, 184776, 184777, 184778 were selected from the ORFs having the nucleotide sequence represented by SEQ ID NO: 174053.

The probes having the nucleotide sequence represented by SEQ ID NOS: 184779, 184780, 184781, 184782, 184783, 184784, 184785, 184786, 184787, 184788, 184789 were selected from the ORFs having the nucleotide sequence represented by SEQ ID NO: 174054.

The probes having the nucleotide sequence represented by SEQ ID NOS: 184790, 184791, 184792, 184793, 184794, 184795, 184796, 184797, 184798, 184799, 184800 were selected from the ORFs having the nucleotide sequence represented by SEQ ID NO: 174055.

The probes having the nucleotide sequence represented by SEQ ID NOS: 184801, 184802, 184803, 184804, 184805, 184806, 184807, 184808, 184809, 184810, 184811 were selected from the ORFs having the nucleotide sequence represented by SEQ ID NO: 174056.

The probes having the nucleotide sequence represented by SEQ ID NOS: 184812, 184813, 184814, 184815, 184816, 184817, 184818, 184819, 184820, 184821, 184822 were selected from the ORFs having the nucleotide sequence represented by SEQ ID NO: 174057.

The probes having the nucleotide sequence represented by SEQ ID NOS: 184823, 184824, 184825, 184826, 184827, 184828, 184829, 184830, 184831, 184832, 184833 were selected from the ORFs having the nucleotide sequence represented by SEQ ID NO: 174058.

The probes having the nucleotide sequence represented by SEQ ID NOS: 184834, 184835, 184836, 184837, 184838, 184839, 184840, 184841, 184842, 184843, 184844 were selected from the ORFs having the nucleotide sequence represented by SEQ ID NO: 174059.

The probes having the nucleotide sequence represented by SEQ ID NOS: 184845, 184846, 184847, 184848, 184849, 184850, 184851, 184852, 184853, 184854, 184855 were selected from the ORFs having the nucleotide sequence represented by SEQ ID NO: 174060.

The probes having the nucleotide sequence represented by SEQ ID NOS: 184856, 184857, 184858, 184859, 184860, 184861, 184862, 184863, 184864, 184865, 184866 were selected from the ORFs having the nucleotide sequence represented by SEQ ID NO: 174061.

The probes having the nucleotide sequence represented by SEQ ID NOS: 184867, 184868, 184869, 184870, 184871, 184872, 184873, 184874, 184875, 184876, 184877 were selected from the ORFs having the nucleotide sequence represented by SEQ ID NO: 174062.

The probes having the nucleotide sequence represented by SEQ ID NOS: 184878, 184879, 184880, 184881, 184882, 184883, 184884, 184885, 184886, 184887, 184888 were selected from the ORFs having the nucleotide sequence represented by SEQ ID NO: 174063.

The probes having the nucleotide sequence represented by SEQ ID NOS: 184889, 184890, 184891, 184892, 184893, 184894, 184895, 184896, 184897, 184898, 184899 were selected from the ORFs having the nucleotide sequence represented by SEQ ID NO: 174064.

The probes having the nucleotide sequence represented by SEQ ID NOS: 184900, 184901, 184902, 184903, 184904, 184905, 184906, 184907, 184908, 184909, 184910 were selected from the ORFs having the nucleotide sequence represented by SEQ ID NO: 174065.

The probes having the nucleotide sequence represented by SEQ ID NOS: 184911, 184912, 184913, 184914, 184915, 184916, 184917, 184918, 184919, 184920, 184921 were selected from the ORFs having the nucleotide sequence represented by SEQ ID NO: 174066.

The probes having the nucleotide sequence represented by SEQ ID NOS: 184922, 184923, 184924, 184925, 184926, 184927, 184928, 184929, 184930, 184931, 184932 were selected from the ORFs having the nucleotide sequence represented by SEQ ID NO: 174067.

The probes having the nucleotide sequence represented by SEQ ID NOS: 184933, 184934, 184935, 184936, 184937, 184938, 184939, 184940, 184941, 184942, 184943 were selected from the ORFs having the nucleotide sequence represented by SEQ ID NO: 174068.

The probes having the nucleotide sequence represented by SEQ ID NOS: 184944, 184945, 184946, 184947, 184948, 184949, 184950, 184951, 184952, 184953, 184954 were selected from the ORFs having the nucleotide sequence represented by SEQ ID NO: 174069.

The probes having the nucleotide sequence represented by SEQ ID NOS: 184955, 184956, 184957, 184958, 184959, 184960, 184961, 184962, 184963, 184964, 184965 were selected from the ORFs having the nucleotide sequence represented by SEQ ID NO: 174070.

The probes having the nucleotide sequence represented by SEQ ID NOS: 184966, 184967, 184968, 184969, 184970, 184971, 184972, 184973, 184974, 184975, 184976 were selected from the ORFs having the nucleotide sequence represented by SEQ ID NO: 174071.

The probes having the nucleotide sequence represented by SEQ ID NOS: 184977, 184978, 184979, 184980, 184981, 184982, 184983, 184984, 184985, 184986, 184987 were selected from the ORFs having the nucleotide sequence represented by SEQ ID NO: 174072.

The probes having the nucleotide sequence represented by SEQ ID NOS: 184988, 184989, 184990, 184991, 184992, 184993, 184994, 184995, 184996, 184997, 184998 were selected from the ORFs having the nucleotide sequence represented by SEQ ID NO: 174073.

The probes having the nucleotide sequence represented by SEQ ID NOS: 184999, 185000, 185001, 185002, 185003, 185004, 185005, 185006, 185007, 185008, 185009 were selected from the ORFs having the nucleotide sequence represented by SEQ ID NO: 174074.

The probes having the nucleotide sequence represented by SEQ ID NOS: 185010, 185011, 185012, 185013, 185014, 185015, 185016, 185017, 185018, 185019, 185020 were selected from the ORFs having the nucleotide sequence represented by SEQ ID NO: 174075.

The probes having the nucleotide sequence represented by SEQ ID NOS: 185021, 185022, 185023, 185024, 185025, 185026, 185027, 185028, 185029, 185030, 185031 were selected from the ORFs having the nucleotide sequence represented by SEQ ID NO: 174076.

The probes having the nucleotide sequence represented by SEQ ID NOS: 185032, 185033, 185034, 185035, 185036, 185037, 185038, 185039, 185040, 185041, 185042 were selected from the ORFs having the nucleotide sequence represented by SEQ ID NO: 174077.

The probes having the nucleotide sequence represented by SEQ ID NOS: 185043, 185044, 185045, 185046, 185047, 185048, 185049, 185050, 185051, 185052, 185053 were selected from the ORFs having the nucleotide sequence represented by SEQ ID NO: 174078.

The probes having the nucleotide sequence represented by SEQ ID NOS: 185054, 185055, 185056, 185057, 185058, 185059, 185060, 185061, 185062, 185063, 185064 were selected from the ORFs having the nucleotide sequence represented by SEQ ID NO: 174079.

The probes having the nucleotide sequence represented by SEQ ID NOS: 185065, 185066, 185067, 185068, 185069, 185070, 185071, 185072, 185073, 185074, 185075 were selected from the ORFs having the nucleotide sequence represented by SEQ ID NO: 174080.

The probes having the nucleotide sequence represented by SEQ ID NOS: 185076, 185077, 185078, 185079, 185080, 185081, 185082, 185083, 185084, 185085, 185086 were selected from the ORFs having the nucleotide sequence represented by SEQ ID NO: 174081.

The probes having the nucleotide sequence represented by SEQ ID NOS: 185087, 185088, 185089, 185090, 185091, 185092, 185093, 185094, 185095, 185096, 185097 were selected from the ORFs having the nucleotide sequence represented by SEQ ID NO: 174082.

The probes having the nucleotide sequence represented by SEQ ID NOS: 185098, 185099, 185100, 185101, 185102, 185103, 185104, 185105, 185106, 185107, 185108 were selected from the ORFs having the nucleotide sequence represented by SEQ ID NO: 174083.

The probes having the nucleotide sequence represented by SEQ ID NOS: 185109, 185110, 185111, 185112, 185113, 185114, 185115, 185116, 185117, 185118, 185119 were selected from the ORFs having the nucleotide sequence represented by SEQ ID NO: 174084.

The probes having the nucleotide sequence represented by SEQ ID NOS: 185120, 185121, 185122, 185123, 185124, 185125, 185126, 185127, 185128, 185129, 185130 were selected from the ORFs having the nucleotide sequence represented by SEQ ID NO: 174085.

The probes having the nucleotide sequence represented by SEQ ID NOS: 185131, 185132, 185133, 185134, 185135, 185136, 185137, 185138, 185139, 185140, 185141 were selected from the ORFs having the nucleotide sequence represented by SEQ ID NO: 174086.

The probes having the nucleotide sequence represented by SEQ ID NOS: 185142, 185143, 185144, 185145, 185146, 185147, 185148, 185149, 185150, 185151, 185152 were selected from the ORFs having the nucleotide sequence represented by SEQ ID NO: 174087.

The probes having the nucleotide sequence represented by SEQ ID NOS: 185153, 185154, 185155, 185156, 185157, 185158, 185159, 185160, 185161, 185162, 185163 were selected from the ORFs having the nucleotide sequence represented by SEQ ID NO: 174088.

The probes having the nucleotide sequence represented by SEQ ID NOS: 185164, 185165, 185166, 185167, 185168, 185169, 185170, 185171, 185172, 185173, 185174 were selected from the ORFs having the nucleotide sequence represented by SEQ ID NO: 174089.

The probes having the nucleotide sequence represented by SEQ ID NOS: 185175, 185176, 185177, 185178, 185179, 185180, 185181, 185182, 185183, 185184, 185185 were selected from the ORFs having the nucleotide sequence represented by SEQ ID NO: 174090.

The probes having the nucleotide sequence represented by SEQ ID NOS: 185186, 185187, 185188, 185189, 185190, 185191, 185192, 185193, 185194, 185195, 185196 were selected from the ORFs having the nucleotide sequence represented by SEQ ID NO: 174091.

The probes having the nucleotide sequence represented by SEQ ID NOS: 185197, 185198, 185199, 185200, 185201, 185202, 185203, 185204, 185205, 185206, 185207 were selected from the ORFs having the nucleotide sequence represented by SEQ ID NO: 174092.

The probes having the nucleotide sequence represented by SEQ ID NOS: 185208, 185209, 185210, 185211, 185212, 185213, 185214, 185215, 185216, 185217, 185218 were selected from the ORFs having the nucleotide sequence represented by SEQ ID NO: 174093.

The probes having the nucleotide sequence represented by SEQ ID NOS: 185219, 185220, 185221, 185222, 185223, 185224, 185225, 185226, 185227, 185228, 185229 were selected from the ORFs having the nucleotide sequence represented by SEQ ID NO: 174094.

The probes having the nucleotide sequence represented by SEQ ID NOS: 185230, 185231, 185232, 185233, 185234, 185235, 185236, 185237, 185238, 185239, 185240 were selected from the ORFs having the nucleotide sequence represented by SEQ ID NO: 174095.

The probes having the nucleotide sequence represented by SEQ ID NOS: 185241, 185242, 185243, 185244, 185245, 185246, 185247, 185248, 185249, 185250, 185251 were selected from the ORFs having the nucleotide sequence represented by SEQ ID NO: 174096.

The probes having the nucleotide sequence represented by SEQ ID NOS: 185252, 185253, 185254, 185255, 185256, 185257, 185258, 185259, 185260, 185261, 185262 were selected from the ORFs having the nucleotide sequence represented by SEQ ID NO: 174097.

The probes having the nucleotide sequence represented by SEQ ID NOS: 185263, 185264, 185265, 185266, 185267, 185268, 185269, 185270, 185271, 185272, 185273 were selected from the ORFs having the nucleotide sequence represented by SEQ ID NO: 174098.

The probes having the nucleotide sequence represented by SEQ ID NOS: 185274, 185275, 185276, 185277, 185278, 185279, 185280, 185281, 185282, 185283, 185284 were selected from the ORFs having the nucleotide sequence represented by SEQ ID NO: 174099.

The probes having the nucleotide sequence represented by SEQ ID NOS: 185285, 185286, 185287, 185288, 185289, 185290, 185291, 185292, 185293, 185294, 185295 were selected from the ORFs having the nucleotide sequence represented by SEQ ID NO: 174100.

The probes having the nucleotide sequence represented by SEQ ID NOS: 185296, 185297, 185298, 185299, 185300, 185301, 185302, 185303, 185304, 185305, 185306 were selected from the ORFs having the nucleotide sequence represented by SEQ ID NO: 174101.

The probes having the nucleotide sequence represented by SEQ ID NOS: 185307, 185308, 185309, 185310, 185311, 185312, 185313, 185314, 185315, 185316, 185317 were selected from the ORFs having the nucleotide sequence represented by SEQ ID NO: 174102.

The probes having the nucleotide sequence represented by SEQ ID NOS: 185318, 185319, 185320, 185321, 185322, 185323, 185324, 185325, 185326, 185327, 185328 were selected from the ORFs having the nucleotide sequence represented by SEQ ID NO: 174103.

The probes having the nucleotide sequence represented by SEQ ID NOS: 185329, 185330, 185331, 185332, 185333, 185334, 185335, 185336, 185337, 185338, 185339 were selected from the ORFs having the nucleotide sequence represented by SEQ ID NO: 174104.

The probes having the nucleotide sequence represented by SEQ ID NOS: 185340, 185341, 185342, 185343, 185344, 185345, 185346, 185347, 185348, 185349, 185350 were selected from the ORFs having the nucleotide sequence represented by SEQ ID NO: 174105.

The probes having the nucleotide sequence represented by SEQ ID NOS: 185351, 185352, 185353, 185354, 185355, 185356, 185357, 185358, 185359, 185360, 185361 were selected from the ORFs having the nucleotide sequence represented by SEQ ID NO: 174106.

The probes having the nucleotide sequence represented by SEQ ID NOS: 185362, 185363, 185364, 185365, 185366, 185367, 185368, 185369, 185370, 185371, 185372 were selected from the ORFs having the nucleotide sequence represented by SEQ ID NO: 174107.

The probes having the nucleotide sequence represented by SEQ ID NOS: 185373, 185374, 185375, 185376, 185377, 185378, 185379, 185380, 185381, 185382, 185383 were selected from the ORFs having the nucleotide sequence represented by SEQ ID NO: 174108.

The probes having the nucleotide sequence represented by SEQ ID NOS: 185384, 185385, 185386, 185387, 185388, 185389, 185390, 185391, 185392, 185393, 185394 were selected from the ORFs having the nucleotide sequence represented by SEQ ID NO: 174109.

The probes having the nucleotide sequence represented by SEQ ID NOS: 185395, 185396, 185397, 185398, 185399, 185400, 185401, 185402, 185403, 185404, 185405 were selected from the ORFs having the nucleotide sequence represented by SEQ ID NO: 174110.

The probes having the nucleotide sequence represented by SEQ ID NOS: 185406, 185407, 185408, 185409, 185410, 185411, 185412, 185413, 185414, 185415, 185416 were selected from the ORFs having the nucleotide sequence represented by SEQ ID NO: 174111.

The probes having the nucleotide sequence represented by SEQ ID NOS: 185417, 185418, 185419, 185420, 185421, 185422, 185423, 185424, 185425, 185426, 185427 were selected from the ORFs having the nucleotide sequence represented by SEQ ID NO: 174112.

The probes having the nucleotide sequence represented by SEQ ID NOS: 185428, 185429, 185430, 185431, 185432, 185433, 185434, 185435, 185436, 185437, 185438 were selected from the ORFs having the nucleotide sequence represented by SEQ ID NO: 174113.

The probes having the nucleotide sequence represented by SEQ ID NOS: 185439, 185440, 185441, 185442, 185443, 185444, 185445, 185446, 185447, 185448, 185449 were selected from the ORFs having the nucleotide sequence represented by SEQ ID NO: 174114.

The probes having the nucleotide sequence represented by SEQ ID NOS: 185450, 185451, 185452, 185453, 185454, 185455, 185456, 185457, 185458, 185459, 185460 were selected from the ORFs having the nucleotide sequence represented by SEQ ID NO: 174115.

The probes having the nucleotide sequence represented by SEQ ID NOS: 185461, 185462, 185463, 185464, 185465, 185466, 185467, 185468, 185469, 185470, 185471 were selected from the ORFs having the nucleotide sequence represented by SEQ ID NO: 174116.

The probes having the nucleotide sequence represented by SEQ ID NOS: 185472, 185473, 185474, 185475, 185476, 185477, 185478, 185479, 185480, 185481, 185482 were selected from the ORFs having the nucleotide sequence represented by SEQ ID NO: 174117.

The probes having the nucleotide sequence represented by SEQ ID NOS: 185483, 185484, 185485, 185486, 185487, 185488, 185489, 185490, 185491, 185492, 185493 were selected from the ORFs having the nucleotide sequence represented by SEQ ID NO: 174118.

The probes having the nucleotide sequence represented by SEQ ID NOS: 185494, 185495, 185496, 185497, 185498, 185499, 185500, 185501, 185502, 185503, 185504 were selected from the ORFs having the nucleotide sequence represented by SEQ ID NO: 174119.

The probes having the nucleotide sequence represented by SEQ ID NOS: 185505, 185506, 185507, 185508, 185509, 185510, 185511, 185512, 185513, 185514, 185515 were selected from the ORFs having the nucleotide sequence represented by SEQ ID NO: 174120.

The probes having the nucleotide sequence represented by SEQ ID NOS: 185516, 185517, 185518, 185519, 185520, 185521, 185522, 185523, 185524, 185525, 185526 were selected from the ORFs having the nucleotide sequence represented by SEQ ID NO: 174121.

The probes having the nucleotide sequence represented by SEQ ID NOS: 185527, 185528, 185529, 185530, 185531, 185532, 185533, 185534, 185535, 185536, 185537 were selected from the ORFs having the nucleotide sequence represented by SEQ ID NO: 174122.

The probes having the nucleotide sequence represented by SEQ ID NOS: 185538, 185539, 185540, 185541, 185542, 185543, 185544, 185545, 185546, 185547, 185548 were selected from the ORFs having the nucleotide sequence represented by SEQ ID NO: 174123.

The probes having the nucleotide sequence represented by SEQ ID NOS: 185549, 185550, 185551, 185552, 185553, 185554, 185555, 185556, 185557, 185558, 185559 were selected from the ORFs having the nucleotide sequence represented by SEQ ID NO: 174124.

The probes having the nucleotide sequence represented by SEQ ID NOS: 185560, 185561, 185562, 185563, 185564, 185565, 185566, 185567, 185568, 185569, 185570 were selected from the ORFs having the nucleotide sequence represented by SEQ ID NO: 174125.

The probes having the nucleotide sequence represented by SEQ ID NOS: 185571, 185572, 185573, 185574, 185575, 185576, 185577, 185578, 185579, 185580, 185581 were selected from the ORFs having the nucleotide sequence represented by SEQ ID NO: 174126.

The probes having the nucleotide sequence represented by SEQ ID NOS: 185582, 185583, 185584, 185585, 185586, 185587, 185588, 185589, 185590, 185591, 185592 were selected from the ORFs having the nucleotide sequence represented by SEQ ID NO: 174127.

The probes having the nucleotide sequence represented by SEQ ID NOS: 185593, 185594, 185595, 185596, 185597, 185598, 185599, 185600, 185601, 185602, 185603 were selected from the ORFs having the nucleotide sequence represented by SEQ ID NO: 174128.

The probes having the nucleotide sequence represented by SEQ ID NOS: 185604, 185605, 185606, 185607, 185608, 185609, 185610, 185611, 185612, 185613, 185614 were selected from the ORFs having the nucleotide sequence represented by SEQ ID NO: 174129.

The probes having the nucleotide sequence represented by SEQ ID NOS: 185615, 185616, 185617, 185618, 185619, 185620, 185621, 185622, 185623, 185624, 185625 were selected from the ORFs having the nucleotide sequence represented by SEQ ID NO: 174130.

The probes having the nucleotide sequence represented by SEQ ID NOS: 185626, 185627, 185628, 185629, 185630, 185631, 185632, 185633, 185634, 185635, 185636 were selected from the ORFs having the nucleotide sequence represented by SEQ ID NO: 174131.

The probes having the nucleotide sequence represented by SEQ ID NOS: 185637, 185638, 185639, 185640, 185641, 185642, 185643, 185644, 185645, 185646, 185647 were selected from the ORFs having the nucleotide sequence represented by SEQ ID NO: 174132.

The probes having the nucleotide sequence represented by SEQ ID NOS: 185648, 185649, 185650, 185651, 185652, 185653, 185654, 185655, 185656, 185657, 185658 were selected from the ORFs having the nucleotide sequence represented by SEQ ID NO: 174133.

The probes having the nucleotide sequence represented by SEQ ID NOS: 185659, 185660, 185661, 185662, 185663, 185664, 185665, 185666, 185667 were selected from the ORFs having the nucleotide sequence represented by SEQ ID NO: 174134.

The probes having the nucleotide sequence represented by SEQ ID NOS: 185668, 185669, 185670, 185671, 185672, 185673, 185674, 185675, 185676, 185677, 185678 were selected from the ORFs having the nucleotide sequence represented by SEQ ID NO: 174135.

The probes having the nucleotide sequence represented by SEQ ID NOS: 185679, 185680, 185681, 185682, 185683, 185684, 185685, 185686, 185687, 185688, 185689 were selected from the ORFs having the nucleotide sequence represented by SEQ ID NO: 174136.

The probes having the nucleotide sequence represented by SEQ ID NOS: 185690, 185691, 185692, 185693, 185694, 185695, 185696, 185697, 185698, 185699, 185700 were selected from the ORFs having the nucleotide sequence represented by SEQ ID NO: 174137.

The probes having the nucleotide sequence represented by SEQ ID NOS: 185701, 185702, 185703, 185704, 185705, 185706, 185707, 185708, 185709, 185710, 185711 were selected from the ORFs having the nucleotide sequence represented by SEQ ID NO: 174138.

The probes having the nucleotide sequence represented by SEQ ID NOS: 185712, 185713, 185714, 185715, 185716, 185717, 185718, 185719, 185720, 185721, 185722 were selected from the ORFs having the nucleotide sequence represented by SEQ ID NO: 174139.

The probes having the nucleotide sequence represented by SEQ ID NOS: 185723, 185724, 185725, 185726, 185727, 185728, 185729, 185730, 185731, 185732, 185733 were selected from the ORFs having the nucleotide sequence represented by SEQ ID NO: 174140.

The probes having the nucleotide sequence represented by SEQ ID NOS: 185734, 185735, 185736, 185737, 185738, 185739, 185740, 185741, 185742, 185743, 185744 were selected from the ORFs having the nucleotide sequence represented by SEQ ID NO: 174141.

The probes having the nucleotide sequence represented by SEQ ID NOS: 185745, 185746, 185747, 185748, 185749, 185750, 185751, 185752 were selected from the ORFs having the nucleotide sequence represented by SEQ ID NO: 174142.

The probes having the nucleotide sequence represented by SEQ ID NOS: 185753, 185754, 185755, 185756, 185757, 185758, 185759, 185760, 185761, 185762, 185763 were selected from the ORFs having the nucleotide sequence represented by SEQ ID NO: 174143.

The probes having the nucleotide sequence represented by SEQ ID NOS: 185764, 185765, 185766, 185767, 185768, 185769, 185770, 185771, 185772, 185773, 185774 were selected from the ORFs having the nucleotide sequence represented by SEQ ID NO: 174144.

The probes having the nucleotide sequence represented by SEQ ID NOS: 185775, 185776, 185777, 185778, 185779, 185780, 185781, 185782, 185783, 185784, 185785 were selected from the ORFs having the nucleotide sequence represented by SEQ ID NO: 174145.

The probes having the nucleotide sequence represented by SEQ ID NOS: 185786, 185787, 185788, 185789, 185790, 185791, 185792, 185793, 185794, 185795, 185796 were selected from the ORFs having the nucleotide sequence represented by SEQ ID NO: 174146.

The probes having the nucleotide sequence represented by SEQ ID NOS: 185797, 185798, 185799, 185800, 185801, 185802, 185803, 185804, 185805, 185806, 185807 were selected from the ORFs having the nucleotide sequence represented by SEQ ID NO: 174147.

The probes having the nucleotide sequence represented by SEQ ID NOS: 185808, 185809, 185810, 185811, 185812, 185813, 185814, 185815, 185816, 185817, 185818 were selected from the ORFs having the nucleotide sequence represented by SEQ ID NO: 174148.

The probes having the nucleotide sequence represented by SEQ ID NOS: 185819, 185820, 185821, 185822, 185823, 185824, 185825, 185826, 185827, 185828, 185829 were selected from the ORFs having the nucleotide sequence represented by SEQ ID NO: 174149.

The probes having the nucleotide sequence represented by SEQ ID NOS: 185830, 185831, 185832, 185833, 185834, 185835, 185836, 185837, 185838, 185839, 185840 were selected from the ORFs having the nucleotide sequence represented by SEQ ID NO: 174150.

The probes having the nucleotide sequence represented by SEQ ID NOS: 185841, 185842, 185843, 185844, 185845, 185846, 185847, 185848, 185849, 185850, 185851 were selected from the ORFs having the nucleotide sequence represented by SEQ ID NO: 174151.

The probes having the nucleotide sequence represented by SEQ ID NOS: 185852, 185853, 185854, 185855, 185856, 185857, 185858, 185859, 185860, 185861, 185862 were selected from the ORFs having the nucleotide sequence represented by SEQ ID NO: 174152.

The probes having the nucleotide sequence represented by SEQ ID NOS: 185863, 185864, 185865, 185866, 185867, 185868, 185869, 185870, 185871, 185872, 185873 were selected from the ORFs having the nucleotide sequence represented by SEQ ID NO: 174153.

The probes having the nucleotide sequence represented by SEQ ID NOS: 185874, 185875, 185876, 185877, 185878, 185879, 185880, 185881, 185882, 185883, 185884 were selected from the ORFs having the nucleotide sequence represented by SEQ ID NO: 174154.

The probes having the nucleotide sequence represented by SEQ ID NOS: 185885, 185886, 185887, 185888, 185889, 185890, 185891, 185892, 185893, 185894, 185895 were selected from the ORFs having the nucleotide sequence represented by SEQ ID NO: 174155.

The probes having the nucleotide sequence represented by SEQ ID NOS: 185896, 185897, 185898, 185899, 185900, 185901, 185902, 185903, 185904, 185905, 185906 were selected from the ORFs having the nucleotide sequence represented by SEQ ID NO: 174156.

The probes having the nucleotide sequence represented by SEQ ID NOS: 185907, 185908, 185909, 185910, 185911, 185912, 185913, 185914, 185915, 185916, 185917 were selected from the ORFs having the nucleotide sequence represented by SEQ ID NO: 174157.

The probes having the nucleotide sequence represented by SEQ ID NOS: 185918, 185919, 185920, 185921, 185922, 185923, 185924, 185925, 185926, 185927, 185928 were selected from the ORFs having the nucleotide sequence represented by SEQ ID NO: 174158.

The probes having the nucleotide sequence represented by SEQ ID NOS: 185929, 185930, 185931, 185932, 185933, 185934, 185935, 185936, 185937, 185938, 185939 were selected from the ORFs having the nucleotide sequence represented by SEQ ID NO: 174159.

The probes having the nucleotide sequence represented by SEQ ID NOS: 185940, 185941, 185942, 185943, 185944, 185945, 185946, 185947, 185948, 185949, 185950 were selected from the ORFs having the nucleotide sequence represented by SEQ ID NO: 174160.

The probes having the nucleotide sequence represented by SEQ ID NOS: 185951, 185952, 185953, 185954, 185955, 185956, 185957, 185958, 185959, 185960, 185961 were selected from the ORFs having the nucleotide sequence represented by SEQ ID NO: 174161.

The probes having the nucleotide sequence represented by SEQ ID NOS: 185962, 185963, 185964, 185965, 185966, 185967, 185968, 185969, 185970, 185971, 185972 were selected from the ORFs having the nucleotide sequence represented by SEQ ID NO: 174162.

The probes having the nucleotide sequence represented by SEQ ID NOS: 185973, 185974, 185975, 185976, 185977, 185978, 185979, 185980, 185981, 185982, 185983 were selected from the ORFs having the nucleotide sequence represented by SEQ ID NO: 174163.

The probes having the nucleotide sequence represented by SEQ ID NOS: 185984, 185985, 185986, 185987, 185988, 185989, 185990, 185991, 185992, 185993, 185994 were selected from the ORFs having the nucleotide sequence represented by SEQ ID NO: 174164.

The probes having the nucleotide sequence represented by SEQ ID NOS: 185995, 185996, 185997, 185998, 185999, 186000, 186001, 186002, 186003, 186004, 186005 were selected from the ORFs having the nucleotide sequence represented by SEQ ID NO: 174165.

The probes having the nucleotide sequence represented by SEQ ID NOS: 186006, 186007, 186008, 186009, 186010, 186011, 186012, 186013, 186014, 186015, 186016 were selected from the ORFs having the nucleotide sequence represented by SEQ ID NO: 174166.

The probes having the nucleotide sequence represented by SEQ ID NOS: 186017, 186018, 186019, 186020, 186021, 186022, 186023, 186024, 186025, 186026, 186027 were selected from the ORFs having the nucleotide sequence represented by SEQ ID NO: 174167.

The probes having the nucleotide sequence represented by SEQ ID NOS: 186028, 186029, 186030, 186031, 186032, 186033, 186034, 186035, 186036, 186037, 186038 were selected from the ORFs having the nucleotide sequence represented by SEQ ID NO: 174168.

The probes having the nucleotide sequence represented by SEQ ID NOS: 186039, 186040, 186041, 186042, 186043, 186044, 186045, 186046, 186047, 186048, 186049 were selected from the ORFs having the nucleotide sequence represented by SEQ ID NO: 174169.

The probes having the nucleotide sequence represented by SEQ ID NOS: 186050, 186051, 186052, 186053, 186054, 186055, 186056, 186057, 186058, 186059, 186060 were selected from the ORFs having the nucleotide sequence represented by SEQ ID NO: 174170.

The probes having the nucleotide sequence represented by SEQ ID NOS: 186061, 186062, 186063, 186064, 186065, 186066, 186067, 186068, 186069, 186070, 186071 were selected from the ORFs having the nucleotide sequence represented by SEQ ID NO: 174171.

The probes having the nucleotide sequence represented by SEQ ID NOS: 186072, 186073, 186074, 186075, 186076, 186077, 186078, 186079, 186080, 186081, 186082 were selected from the ORFs having the nucleotide sequence represented by SEQ ID NO: 174172.

The probes having the nucleotide sequence represented by SEQ ID NOS: 186083, 186084, 186085, 186086, 186087, 186088, 186089, 186090, 186091, 186092, 186093 were selected from the ORFs having the nucleotide sequence represented by SEQ ID NO: 174173.

The probes having the nucleotide sequence represented by SEQ ID NOS: 186094, 186095, 186096, 186097, 186098, 186099, 186100, 186101, 186102, 186103, 186104 were selected from the ORFs having the nucleotide sequence represented by SEQ ID NO: 174174.

The probes having the nucleotide sequence represented by SEQ ID NOS: 186105, 186106, 186107, 186108, 186109, 186110, 186111, 186112, 186113, 186114, 186115 were selected from the ORFs having the nucleotide sequence represented by SEQ ID NO: 174175.

The probes having the nucleotide sequence represented by SEQ ID NOS: 186116, 186117, 186118, 186119, 186120, 186121, 186122, 186123, 186124, 186125, 186126 were selected from the ORFs having the nucleotide sequence represented by SEQ ID NO: 174176.

The probes having the nucleotide sequence represented by SEQ ID NOS: 186127, 186128, 186129, 186130, 186131, 186132, 186133, 186134, 186135, 186136, 186137 were selected from the ORFs having the nucleotide sequence represented by SEQ ID NO: 174177.

The probes having the nucleotide sequence represented by SEQ ID NOS: 186138, 186139, 186140, 186141, 186142, 186143, 186144, 186145, 186146, 186147, 186148 were selected from the ORFs having the nucleotide sequence represented by SEQ ID NO: 174178.

The probes having the nucleotide sequence represented by SEQ ID NOS: 186149, 186150, 186151, 186152, 186153, 186154, 186155, 186156, 186157, 186158, 186159 were selected from the ORFs having the nucleotide sequence represented by SEQ ID NO: 174179.

The probes having the nucleotide sequence represented by SEQ ID NOS: 186160, 186161, 186162, 186163, 186164, 186165, 186166, 186167, 186168, 186169, 186170 were selected from the ORFs having the nucleotide sequence represented by SEQ ID NO: 174180.

The probes having the nucleotide sequence represented by SEQ ID NOS: 186171, 186172, 186173, 186174, 186175, 186176, 186177, 186178, 186179 were selected from the ORFs having the nucleotide sequence represented by SEQ ID NO: 174181.

The probes having the nucleotide sequence represented by SEQ ID NOS: 186180, 186181, 186182, 186183, 186184, 186185, 186186, 186187, 186188, 186189, 186190 were selected from the ORFs having the nucleotide sequence represented by SEQ ID NO: 174182.

The probes having the nucleotide sequence represented by SEQ ID NOS: 186191, 186192, 186193, 186194, 186195, 186196, 186197, 186198, 186199, 186200, 186201 were selected from the ORFs having the nucleotide sequence represented by SEQ ID NO: 174183.

The probes having the nucleotide sequence represented by SEQ ID NOS: 186202, 186203, 186204, 186205, 186206, 186207, 186208, 186209, 186210, 186211, 186212 were selected from the ORFs having the nucleotide sequence represented by SEQ ID NO: 174184.

The probes having the nucleotide sequence represented by SEQ ID NOS: 186213, 186214, 186215, 186216, 186217, 186218, 186219, 186220, 186221, 186222, 186223 were selected from the ORFs having the nucleotide sequence represented by SEQ ID NO: 174185.

The probes having the nucleotide sequence represented by SEQ ID NOS: 186224, 186225, 186226, 186227, 186228, 186229, 186230, 186231, 186232, 186233, 186234 were selected from the ORFs having the nucleotide sequence represented by SEQ ID NO: 174186.

The probes having the nucleotide sequence represented by SEQ ID NOS: 186235, 186236, 186237, 186238, 186239, 186240, 186241, 186242, 186243, 186244, 186245 were selected from the ORFs having the nucleotide sequence represented by SEQ ID NO: 174187.

The probes having the nucleotide sequence represented by SEQ ID NOS: 186246, 186247, 186248, 186249, 186250, 186251, 186252, 186253, 186254, 186255, 186256 were selected from the ORFs having the nucleotide sequence represented by SEQ ID NO: 174188.

The probes having the nucleotide sequence represented by SEQ ID NOS: 186257, 186258, 186259, 186260, 186261, 186262, 186263, 186264, 186265, 186266, 186267 were selected from the ORFs having the nucleotide sequence represented by SEQ ID NO: 174189.

The probes having the nucleotide sequence represented by SEQ ID NOS: 186268, 186269, 186270, 186271, 186272, 186273, 186274, 186275, 186276, 186277, 186278 were selected from the ORFs having the nucleotide sequence represented by SEQ ID NO: 174190.

The probes having the nucleotide sequence represented by SEQ ID NOS: 186279, 186280, 186281, 186282, 186283, 186284, 186285, 186286, 186287, 186288, 186289 were selected from the ORFs having the nucleotide sequence represented by SEQ ID NO: 174191.

The probes having the nucleotide sequence represented by SEQ ID NOS: 186290, 186291, 186292, 186293, 186294, 186295, 186296, 186297, 186298, 186299, 186300 were selected from the ORFs having the nucleotide sequence represented by SEQ ID NO: 174192.

The probes having the nucleotide sequence represented by SEQ ID NOS: 186301, 186302, 186303, 186304, 186305, 186306, 186307, 186308, 186309, 186310, 186311 were selected from the ORFs having the nucleotide sequence represented by SEQ ID NO: 174193.

The probes having the nucleotide sequence represented by SEQ ID NOS: 186312, 186313, 186314, 186315, 186316, 186317, 186318, 186319, 186320, 186321, 186322 were selected from the ORFs having the nucleotide sequence represented by SEQ ID NO: 174194.

The probes having the nucleotide sequence represented by SEQ ID NOS: 186323, 186324, 186325, 186326, 186327, 186328, 186329, 186330, 186331, 186332, 186333 were selected from the ORFs having the nucleotide sequence represented by SEQ ID NO: 174195.

The probes having the nucleotide sequence represented by SEQ ID NOS: 186334, 186335, 186336, 186337, 186338, 186339, 186340, 186341 were selected from the ORFs having the nucleotide sequence represented by SEQ ID NO: 174196.

The probes having the nucleotide sequence represented by SEQ ID NOS: 186342, 186343, 186344, 186345, 186346, 186347, 186348, 186349, 186350, 186351, 186352 were selected from the ORFs having the nucleotide sequence represented by SEQ ID NO: 174197.

The probes having the nucleotide sequence represented by SEQ ID NOS: 186353, 186354, 186355, 186356, 186357, 186358, 186359, 186360, 186361, 186362, 186363 were selected from the ORFs having the nucleotide sequence represented by SEQ ID NO: 174198.

The probes having the nucleotide sequence represented by SEQ ID NOS: 186364, 186365, 186366, 186367, 186368, 186369, 186370, 186371, 186372, 186373, 186374 were selected from the ORFs having the nucleotide sequence represented by SEQ ID NO: 174199.

The probes having the nucleotide sequence represented by SEQ ID NOS: 186375, 186376, 186377, 186378, 186379, 186380, 186381, 186382, 186383, 186384, 186385 were selected from the ORFs having the nucleotide sequence represented by SEQ ID NO: 174200.

The probes having the nucleotide sequence represented by SEQ ID NOS: 186386, 186387, 186388, 186389, 186390, 186391, 186392, 186393, 186394, 186395, 186396 were selected from the ORFs having the nucleotide sequence represented by SEQ ID NO: 174201.

The probes having the nucleotide sequence represented by SEQ ID NOS: 186397, 186398, 186399, 186400, 186401, 186402, 186403, 186404, 186405, 186406, 186407 were selected from the ORFs having the nucleotide sequence represented by SEQ ID NO: 174202.

The probes having the nucleotide sequence represented by SEQ ID NOS: 186408, 186409, 186410, 186411, 186412, 186413, 186414, 186415, 186416, 186417, 186418 were selected from the ORFs having the nucleotide sequence represented by SEQ ID NO: 174203.

The probes having the nucleotide sequence represented by SEQ ID NOS: 186419, 186420, 186421, 186422, 186423, 186424, 186425, 186426, 186427, 186428, 186429 were selected from the ORFs having the nucleotide sequence represented by SEQ ID NO: 174204.

The probes having the nucleotide sequence represented by SEQ ID NOS: 186430, 186431, 186432, 186433, 186434, 186435, 186436, 186437, 186438, 186439, 186440 were selected from the ORFs having the nucleotide sequence represented by SEQ ID NO: 174205.

The probes having the nucleotide sequence represented by SEQ ID NOS: 186441, 186442, 186443, 186444, 186445, 186446, 186447, 186448, 186449, 186450, 186451 were selected from the ORFs having the nucleotide sequence represented by SEQ ID NO: 174206.

The probes having the nucleotide sequence represented by SEQ ID NOS: 186452, 186453, 186454, 186455, 186456, 186457, 186458, 186459, 186460, 186461, 186462 were selected from the ORFs having the nucleotide sequence represented by SEQ ID NO: 174207.

The probes having the nucleotide sequence represented by SEQ ID NOS: 186463, 186464, 186465, 186466, 186467, 186468, 186469, 186470, 186471, 186472, 186473 were selected from the ORFs having the nucleotide sequence represented by SEQ ID NO: 174208.

The probes having the nucleotide sequence represented by SEQ ID NOS: 186474, 186475, 186476, 186477, 186478, 186479, 186480, 186481, 186482, 186483, 186484 were selected from the ORFs having the nucleotide sequence represented by SEQ ID NO: 174209.

The probes having the nucleotide sequence represented by SEQ ID NOS: 186485, 186486, 186487, 186488, 186489, 186490, 186491, 186492, 186493, 186494, 186495 were selected from the ORFs having the nucleotide sequence represented by SEQ ID NO: 174210.

The probes having the nucleotide sequence represented by SEQ ID NOS: 186496, 186497, 186498, 186499, 186500, 186501, 186502, 186503, 186504, 186505, 186506 were selected from the ORFs having the nucleotide sequence represented by SEQ ID NO: 174211.

The probes having the nucleotide sequence represented by SEQ ID NOS: 186507, 186508, 186509, 186510, 186511, 186512, 186513, 186514, 186515, 186516, 186517 were selected from the ORFs having the nucleotide sequence represented by SEQ ID NO: 174212.

The probes having the nucleotide sequence represented by SEQ ID NOS: 186518, 186519, 186520, 186521, 186522, 186523, 186524, 186525, 186526, 186527, 186528 were selected from the ORFs having the nucleotide sequence represented by SEQ ID NO: 174213.

The probes having the nucleotide sequence represented by SEQ ID NOS: 186529, 186530, 186531, 186532, 186533, 186534, 186535, 186536, 186537, 186538, 186539 were selected from the ORFs having the nucleotide sequence represented by SEQ ID NO: 174214.

The probes having the nucleotide sequence represented by SEQ ID NOS: 186540, 186541, 186542, 186543, 186544, 186545, 186546, 186547, 186548, 186549, 186550 were selected from the ORFs having the nucleotide sequence represented by SEQ ID NO: 174215.

The probes having the nucleotide sequence represented by SEQ ID NOS: 186551, 186552, 186553, 186554, 186555, 186556, 186557, 186558, 186559, 186560, 186561 were selected from the ORFs having the nucleotide sequence represented by SEQ ID NO: 174216.

The probes having the nucleotide sequence represented by SEQ ID NOS: 186562, 186563, 186564, 186565, 186566, 186567, 186568, 186569, 186570, 186571, 186572 were selected from the ORFs having the nucleotide sequence represented by SEQ ID NO: 174217.

The probes having the nucleotide sequence represented by SEQ ID NOS: 186573, 186574, 186575, 186576, 186577, 186578, 186579, 186580, 186581, 186582, 186583 were selected from the ORFs having the nucleotide sequence represented by SEQ ID NO: 174218.

The probes having the nucleotide sequence represented by SEQ ID NOS: 186584, 186585, 186586, 186587, 186588, 186589, 186590, 186591, 186592, 186593, 186594 were selected from the ORFs having the nucleotide sequence represented by SEQ ID NO: 174219.

The probes having the nucleotide sequence represented by SEQ ID NOS: 186595, 186596, 186597, 186598, 186599, 186600, 186601, 186602, 186603, 186604, 186605 were selected from the ORFs having the nucleotide sequence represented by SEQ ID NO: 174220.

The probes having the nucleotide sequence represented by SEQ ID NOS: 186606, 186607, 186608, 186609, 186610, 186611, 186612, 186613, 186614, 186615, 186616 were selected from the ORFs having the nucleotide sequence represented by SEQ ID NO: 174221.

The probes having the nucleotide sequence represented by SEQ ID NOS: 186617, 186618, 186619, 186620, 186621, 186622, 186623, 186624, 186625, 186626, 186627 were selected from the ORFs having the nucleotide sequence represented by SEQ ID NO: 174222.

The probes having the nucleotide sequence represented by SEQ ID NOS: 186628, 186629, 186630, 186631, 186632, 186633, 186634, 186635, 186636, 186637, 186638 were selected from the ORFs having the nucleotide sequence represented by SEQ ID NO: 174223.

The probes having the nucleotide sequence represented by SEQ ID NOS: 186639, 186640, 186641, 186642, 186643, 186644, 186645, 186646, 186647, 186648, 186649 were selected from the ORFs having the nucleotide sequence represented by SEQ ID NO: 174224.

The probes having the nucleotide sequence represented by SEQ ID NOS: 186650, 186651, 186652, 186653, 186654, 186655, 186656, 186657, 186658, 186659, 186660 were selected from the ORFs having the nucleotide sequence represented by SEQ ID NO: 174225.

The probes having the nucleotide sequence represented by SEQ ID NOS: 186661, 186662, 186663, 186664, 186665, 186666, 186667, 186668, 186669, 186670, 186671 were selected from the ORFs having the nucleotide sequence represented by SEQ ID NO: 174226.

The probes having the nucleotide sequence represented by SEQ ID NOS: 186672, 186673, 186674, 186675, 186676, 186677, 186678, 186679, 186680, 186681, 186682 were selected from the ORFs having the nucleotide sequence represented by SEQ ID NO: 174227.

The probes having the nucleotide sequence represented by SEQ ID NOS: 186683, 186684, 186685, 186686, 186687, 186688, 186689, 186690, 186691, 186692, 186693 were selected from the ORFs having the nucleotide sequence represented by SEQ ID NO: 174228.

The probes having the nucleotide sequence represented by SEQ ID NOS: 186694, 186695, 186696, 186697, 186698, 186699, 186700, 186701, 186702, 186703, 186704 were selected from the ORFs having the nucleotide sequence represented by SEQ ID NO: 174229.

The probes having the nucleotide sequence represented by SEQ ID NOS: 186705, 186706, 186707, 186708, 186709, 186710, 186711, 186712, 186713, 186714, 186715 were selected from the ORFs having the nucleotide sequence represented by SEQ ID NO: 174230.

The probes having the nucleotide sequence represented by SEQ ID NOS: 186716, 186717, 186718, 186719, 186720, 186721, 186722, 186723, 186724, 186725, 186726 were selected from the ORFs having the nucleotide sequence represented by SEQ ID NO: 174231.

The probes having the nucleotide sequence represented by SEQ ID NOS: 186727, 186728, 186729, 186730, 186731, 186732, 186733, 186734, 186735, 186736, 186737 were selected from the ORFs having the nucleotide sequence represented by SEQ ID NO: 174232.

The probes having the nucleotide sequence represented by SEQ ID NOS: 186738, 186739, 186740, 186741, 186742, 186743, 186744, 186745, 186746, 186747, 186748 were selected from the ORFs having the nucleotide sequence represented by SEQ ID NO: 174233.

The probes having the nucleotide sequence represented by SEQ ID NOS: 186749, 186750, 186751, 186752, 186753, 186754, 186755, 186756, 186757, 186758, 186759 were selected from the ORFs having the nucleotide sequence represented by SEQ ID NO: 174234.

The probes having the nucleotide sequence represented by SEQ ID NOS: 186760, 186761, 186762, 186763, 186764, 186765, 186766, 186767, 186768, 186769, 186770 were selected from the ORFs having the nucleotide sequence represented by SEQ ID NO: 174235.

The probes having the nucleotide sequence represented by SEQ ID NOS: 186771, 186772, 186773, 186774, 186775, 186776, 186777, 186778, 186779, 186780, 186781 were selected from the ORFs having the nucleotide sequence represented by SEQ ID NO: 174236.

The probes having the nucleotide sequence represented by SEQ ID NOS: 186782, 186783, 186784, 186785, 186786, 186787, 186788, 186789, 186790, 186791, 186792 were selected from the ORFs having the nucleotide sequence represented by SEQ ID NO: 174237.

The probes having the nucleotide sequence represented by SEQ ID NOS: 186793, 186794, 186795, 186796, 186797, 186798, 186799, 186800, 186801, 186802, 186803 were selected from the ORFs having the nucleotide sequence represented by SEQ ID NO: 174238.

The probes having the nucleotide sequence represented by SEQ ID NOS: 186804, 186805, 186806, 186807, 186808, 186809, 186810, 186811, 186812, 186813, 186814 were selected from the ORFs having the nucleotide sequence represented by SEQ ID NO: 174239.

The probes having the nucleotide sequence represented by SEQ ID NOS: 186815, 186816, 186817, 186818, 186819, 186820, 186821, 186822, 186823, 186824, 186825 were selected from the ORFs having the nucleotide sequence represented by SEQ ID NO: 174240.

The probes having the nucleotide sequence represented by SEQ ID NOS: 186826, 186827, 186828, 186829, 186830, 186831, 186832, 186833, 186834, 186835, 186836 were selected from the ORFs having the nucleotide sequence represented by SEQ ID NO: 174241.

The probes having the nucleotide sequence represented by SEQ ID NOS: 186837, 186838, 186839, 186840, 186841, 186842, 186843, 186844, 186845, 186846, 186847 were selected from the ORFs having the nucleotide sequence represented by SEQ ID NO: 174242.

The probes having the nucleotide sequence represented by SEQ ID NOS: 186848, 186849, 186850, 186851, 186852, 186853, 186854, 186855, 186856, 186857, 186858 were selected from the ORFs having the nucleotide sequence represented by SEQ ID NO: 174243.

The probes having the nucleotide sequence represented by SEQ ID NOS: 186859, 186860, 186861, 186862, 186863, 186864, 186865, 186866, 186867, 186868, 186869 were selected from the ORFs having the nucleotide sequence represented by SEQ ID NO: 174244.

The probes having the nucleotide sequence represented by SEQ ID NOS: 186870, 186871, 186872, 186873, 186874, 186875, 186876, 186877, 186878, 186879, 186880 were selected from the ORFs having the nucleotide sequence represented by SEQ ID NO: 174245.

The probes having the nucleotide sequence represented by SEQ ID NOS: 186881, 186882, 186883, 186884, 186885, 186886, 186887, 186888, 186889, 186890, 186891 were selected from the ORFs having the nucleotide sequence represented by SEQ ID NO: 174246.

The probes having the nucleotide sequence represented by SEQ ID NOS: 186892, 186893, 186894, 186895, 186896, 186897, 186898, 186899, 186900, 186901, 186902 were selected from the ORFs having the nucleotide sequence represented by SEQ ID NO: 174247.

The probes having the nucleotide sequence represented by SEQ ID NOS: 186903, 186904, 186905, 186906, 186907, 186908, 186909, 186910, 186911, 186912, 186913 were selected from the ORFs having the nucleotide sequence represented by SEQ ID NO: 174248.

The probes having the nucleotide sequence represented by SEQ ID NOS: 186914, 186915, 186916, 186917, 186918, 186919, 186920, 186921, 186922, 186923, 186924 were selected from the ORFs having the nucleotide sequence represented by SEQ ID NO: 174249.

The probes having the nucleotide sequence represented by SEQ ID NOS: 186925, 186926, 186927, 186928, 186929, 186930, 186931, 186932, 186933, 186934, 186935 were selected from the ORFs having the nucleotide sequence represented by SEQ ID NO: 174250.

The probes having the nucleotide sequence represented by SEQ ID NOS: 186936, 186937, 186938, 186939, 186940, 186941, 186942, 186943, 186944 were selected from the ORFs having the nucleotide sequence represented by SEQ ID NO: 174251.

The probes having the nucleotide sequence represented by SEQ ID NOS: 186945, 186946, 186947, 186948, 186949, 186950, 186951, 186952, 186953, 186954, 186955 were selected from the ORFs having the nucleotide sequence represented by SEQ ID NO: 174252.

The probes having the nucleotide sequence represented by SEQ ID NOS: 186956, 186957, 186958, 186959, 186960, 186961, 186962, 186963, 186964, 186965, 186966 were selected from the ORFs having the nucleotide sequence represented by SEQ ID NO: 174253.

The probes having the nucleotide sequence represented by SEQ ID NOS: 186967, 186968, 186969, 186970, 186971, 186972, 186973, 186974, 186975, 186976, 186977 were selected from the ORFs having the nucleotide sequence represented by SEQ ID NO: 174254.

The probes having the nucleotide sequence represented by SEQ ID NOS: 186978, 186979, 186980, 186981, 186982, 186983, 186984, 186985, 186986, 186987, 186988 were selected from the ORFs having the nucleotide sequence represented by SEQ ID NO: 174255.

The probes having the nucleotide sequence represented by SEQ ID NOS: 186989, 186990, 186991, 186992, 186993, 186994, 186995, 186996, 186997, 186998, 186999 were selected from the ORFs having the nucleotide sequence represented by SEQ ID NO: 174256.

The probes having the nucleotide sequence represented by SEQ ID NOS: 187000, 187001, 187002, 187003, 187004, 187005, 187006, 187007, 187008, 187009, 187010 were selected from the ORFs having the nucleotide sequence represented by SEQ ID NO: 174257.

The probes having the nucleotide sequence represented by SEQ ID NOS: 187011, 187012, 187013, 187014, 187015, 187016, 187017, 187018, 187019, 187020, 187021 were selected from the ORFs having the nucleotide sequence represented by SEQ ID NO: 174258.

The probes having the nucleotide sequence represented by SEQ ID NOS: 187022, 187023, 187024, 187025, 187026, 187027, 187028, 187029, 187030, 187031, 187032 were selected from the ORFs having the nucleotide sequence represented by SEQ ID NO: 174259.

The probes having the nucleotide sequence represented by SEQ ID NOS: 187033, 187034, 187035, 187036, 187037, 187038, 187039, 187040, 187041, 187042, 187043 were selected from the ORFs having the nucleotide sequence represented by SEQ ID NO: 174260.

The probes having the nucleotide sequence represented by SEQ ID NOS: 187044, 187045, 187046, 187047, 187048, 187049, 187050, 187051, 187052, 187053, 187054 were selected from the ORFs having the nucleotide sequence represented by SEQ ID NO: 174261.

The probes having the nucleotide sequence represented by SEQ ID NOS: 187055, 187056, 187057, 187058, 187059, 187060, 187061, 187062, 187063, 187064, 187065 were selected from the ORFs having the nucleotide sequence represented by SEQ ID NO: 174262.

The probes having the nucleotide sequence represented by SEQ ID NOS: 187066, 187067, 187068, 187069, 187070, 187071, 187072, 187073, 187074, 187075, 187076 were selected from the ORFs having the nucleotide sequence represented by SEQ. ID NO: 174263.

The probes having the nucleotide sequence represented by SEQ ID NOS: 187077, 187078, 187079, 187080, 187081, 187082, 187083, 187084, 187085, 187086, 187087 were selected from the ORFs having the nucleotide sequence represented by SEQ ID NO: 174264.

The probes having the nucleotide sequence represented by SEQ ID NOS: 187088, 187089, 187090, 187091, 187092, 187093, 187094, 187095, 187096, 187097, 187098 were selected from the ORFs having the nucleotide sequence represented by SEQ ID NO: 174265.

The probes having the nucleotide sequence represented by SEQ ID NOS: 187099, 187100, 187101, 187102, 187103, 187104, 187105, 187106, 187107, 187108, 187109 were selected from the ORFs having the nucleotide sequence represented by SEQ ID NO: 174266.

The probes having the nucleotide sequence represented by SEQ ID NOS: 187110, 187111, 187112, 187113, 187114, 187115, 187116, 187117, 187118, 187119, 187120 were selected from the ORFs having the nucleotide sequence represented by SEQ ID NO: 174267.

The probes having the nucleotide sequence represented by SEQ ID NOS: 187121, 187122, 187123, 187124, 187125, 187126, 187127, 187128, 187129, 187130, 187131 were selected from the ORFs having the nucleotide sequence represented by SEQ ID NO: 174268.

The probes having the nucleotide sequence represented by SEQ ID NOS: 187132, 187133, 187134, 187135, 187136, 187137, 187138, 187139, 187140, 187141, 187142 were selected from the ORFs having the nucleotide sequence represented by SEQ ID NO: 174269.

The probes having the nucleotide sequence represented by SEQ ID NOS: 187143, 187144, 187145, 187146, 187147, 187148, 187149, 187150, 187151, 187152, 187153 were selected from the ORFs having the nucleotide sequence represented by SEQ ID NO: 174270.

The probes having the nucleotide sequence represented by SEQ ID NOS: 187154, 187155, 187156, 187157, 187158, 187159, 187160, 187161, 187162, 187163, 187164 were selected from the ORFs having the nucleotide sequence represented by SEQ ID NO: 174271.

The probes having the nucleotide sequence represented by SEQ ID NOS: 187165, 187166, 187167, 187168, 187169, 187170, 187171, 187172, 187173, 187174, 187175 were selected from the ORFs having the nucleotide sequence represented by SEQ ID NO: 174272.

The probes having the nucleotide sequence represented by SEQ ID NOS: 187176, 187177, 187178, 187179, 187180, 187181, 187182, 187183, 187184, 187185, 187186 were selected from the ORFs having the nucleotide sequence represented by SEQ ID NO: 174273.

The probes having the nucleotide sequence represented by SEQ ID NOS: 187187, 187188, 187189, 187190, 187191, 187192, 187193, 187194, 187195, 187196, 187197 were selected from the ORFs having the nucleotide sequence represented by SEQ ID NO: 174274.

The probes having the nucleotide sequence represented by SEQ ID NOS: 187198, 187199, 187200, 187201, 187202, 187203, 187204, 187205, 187206, 187207, 187208 were selected from the ORFs having the nucleotide sequence represented by SEQ ID NO: 174275.

The probes having the nucleotide sequence represented by SEQ ID NOS: 187209, 187210, 187211, 187212, 187213, 187214, 187215, 187216, 187217, 187218, 187219 were selected from the ORFs having the nucleotide sequence represented by SEQ ID NO: 174276.

The probes having the nucleotide sequence represented by SEQ ID NOS: 187220, 187221, 187222, 187223, 187224, 187225, 187226, 187227, 187228, 187229, 187230 were selected from the ORFs having the nucleotide sequence represented by SEQ ID NO: 174277.

The probes having the nucleotide sequence represented by SEQ ID NOS: 187231, 187232, 187233, 187234, 187235, 187236, 187237, 187238, 187239, 187240, 187241 were selected from the ORFs having the nucleotide sequence represented by SEQ ID NO: 174278.

The probes having the nucleotide sequence represented by SEQ ID NOS: 187242, 187243, 187244, 187245, 187246, 187247, 187248, 187249, 187250, 187251, 187252 were selected from the ORFs having the nucleotide sequence represented by SEQ ID NO: 174279.

The probes having the nucleotide sequence represented by SEQ ID NOS: 187253, 187254, 187255, 187256, 187257, 187258, 187259, 187260, 187261, 187262, 187263 were selected from the ORFs having the nucleotide sequence represented by SEQ ID NO: 174280.

The probes having the nucleotide sequence represented by SEQ ID NOS: 187264, 187265, 187266, 187267, 187268, 187269, 187270, 187271, 187272, 187273, 187274 were selected from the ORFs having the nucleotide sequence represented by SEQ ID NO: 174281.

The probes having the nucleotide sequence represented by SEQ ID NOS: 187275, 187276, 187277, 187278, 187279, 187280, 187281, 187282, 187283, 187284, 187285 were selected from the ORFs having the nucleotide sequence represented by SEQ ID NO: 174282.

The probes having the nucleotide sequence represented by SEQ ID NOS: 187286, 187287, 187288, 187289, 187290, 187291, 187292, 187293, 187294, 187295, 187296 were selected from the ORFs having the nucleotide sequence represented by SEQ ID NO: 174283.

The probes having the nucleotide sequence represented by SEQ ID NOS: 187297, 187298, 187299, 187300, 187301, 187302, 187303, 187304, 187305, 187306, 187307 were selected from the ORFs having the nucleotide sequence represented by SEQ ID NO: 174284.

The probes having the nucleotide sequence represented by SEQ ID NOS: 187308, 187309, 187310, 187311, 187312, 187313, 187314, 187315, 187316, 187317, 187318 were selected from the ORFs having the nucleotide sequence represented by SEQ ID NO: 174285.

The probes having the nucleotide sequence represented by SEQ ID NOS: 187319, 187320, 187321, 187322, 187323, 187324, 187325, 187326, 187327, 187328, 187329 were selected from the ORFs having the nucleotide sequence represented by SEQ ID NO: 174286.

The probes having the nucleotide sequence represented by SEQ ID NOS: 187330, 187331, 187332, 187333, 187334, 187335, 187336, 187337, 187338, 187339, 187340 were selected from the ORFs having the nucleotide sequence represented by SEQ ID NO: 174287.

The probes having the nucleotide sequence represented by SEQ ID NOS: 187341, 187342, 187343, 187344, 187345, 187346, 187347, 187348, 187349, 187350, 187351 were selected from the ORFs having the nucleotide sequence represented by SEQ ID NO: 174288.

The probes having the nucleotide sequence represented by SEQ ID NOS: 187352, 187353, 187354, 187355, 187356, 187357, 187358, 187359, 187360, 187361, 187362 were selected from the ORFs having the nucleotide sequence represented by SEQ ID NO: 174289.

The probes having the nucleotide sequence represented by SEQ ID NOS: 187363, 187364, 187365, 187366, 187367, 187368, 187369, 187370, 187371, 187372, 187373 were selected from the ORFs having the nucleotide sequence represented by SEQ ID NO: 174290.

The probes having the nucleotide sequence represented by SEQ ID NOS: 187374, 187375, 187376, 187377, 187378, 187379, 187380, 187381, 187382, 187383, 187384 were selected from the ORFs having the nucleotide sequence represented by SEQ ID NO: 174291.

The probes having the nucleotide sequence represented by SEQ ID NOS: 187385, 187386, 187387, 187388, 187389, 187390, 187391, 187392, 187393, 187394, 187395 were selected from the ORFs having the nucleotide sequence represented by SEQ ID NO: 174292.

The probes having the nucleotide sequence represented by SEQ ID NOS: 187396, 187397, 187398, 187399, 187400, 187401, 187402, 187403, 187404, 187405, 187406 were selected from the ORFs having the nucleotide sequence represented by SEQ ID NO: 174293.

The probes having the nucleotide sequence represented by SEQ ID NOS: 187407, 187408, 187409, 187410, 187411, 187412, 187413, 187414, 187415, 187416, 187417 were selected from the ORFs having the nucleotide sequence represented by SEQ ID NO: 174294.

The probes having the nucleotide sequence represented by SEQ ID NOS: 187418, 187419, 187420, 187421, 187422, 187423, 187424, 187425, 187426, 187427, 187428 were selected from the ORFs having the nucleotide sequence represented by SEQ ID NO: 174295.

The probes having the nucleotide sequence represented by SEQ ID NOS: 187429, 187430, 187431, 187432, 187433, 187434, 187435, 187436, 187437, 187438, 187439 were selected from the ORFs having the nucleotide sequence represented by SEQ ID NO: 174296.

The probes having the nucleotide sequence represented by SEQ ID NOS: 187440, 187441, 187442, 187443, 187444, 187445, 187446, 187447, 187448, 187449, 187450 were selected from the ORFs having the nucleotide sequence represented by SEQ ID NO: 174297.

The probes having the nucleotide sequence represented by SEQ ID NOS: 187451, 187452, 187453, 187454, 187455, 187456, 187457, 187458, 187459, 187460, 187461 were selected from the ORFs having the nucleotide sequence represented by SEQ ID NO: 174298.

The probes having the nucleotide sequence represented by SEQ ID NOS: 187462, 187463, 187464, 187465, 187466, 187467, 187468, 187469, 187470, 187471, 187472 were selected from the ORFs having the nucleotide sequence represented by SEQ ID NO: 174299.

The probes having the nucleotide sequence represented by SEQ ID NOS: 187473, 187474, 187475, 187476, 187477, 187478, 187479, 187480, 187481, 187482, 187483 were selected from the ORFs having the nucleotide sequence represented by SEQ ID NO: 174300.

The probes having the nucleotide sequence represented by SEQ ID NOS: 187484, 187485, 187486, 187487, 187488, 187489, 187490, 187491, 187492, 187493, 187494 were selected from the ORFs having the nucleotide sequence represented by SEQ ID NO: 174301.

The probes having the nucleotide sequence represented by SEQ ID NOS: 187495, 187496, 187497, 187498, 187499, 187500, 187501, 187502, 187503, 187504, 187505 were selected from the ORFs having the nucleotide sequence represented by SEQ ID NO: 174302.

The probes having the nucleotide sequence represented by SEQ ID NOS: 187506, 187507, 187508, 187509, 187510, 187511, 187512, 187513, 187514, 187515, 187516 were selected from the ORFs having the nucleotide sequence represented by SEQ ID NO: 174303.

The probes having the nucleotide sequence represented by SEQ ID NOS: 187517, 187518, 187519, 187520, 187521, 187522, 187523, 187524, 187525, 187526, 187527 were selected from the ORFs having the nucleotide sequence represented by SEQ ID NO: 174304.

The probes having the nucleotide sequence represented by SEQ ID NOS: 187528, 187529, 187530, 187531, 187532, 187533, 187534, 187535, 187536, 187537, 187538 were selected from the ORFs having the nucleotide sequence represented by SEQ ID NO: 174305.

The probes having the nucleotide sequence represented by SEQ ID NOS: 187539, 187540, 187541, 187542, 187543, 187544, 187545, 187546, 187547, 187548, 187549 were selected from the ORFs having the nucleotide sequence represented by SEQ ID NO: 174306.

The probes having the nucleotide sequence represented by SEQ ID NOS: 187550, 187551, 187552, 187553, 187554, 187555, 187556, 187557, 187558, 187559, 187560 were selected from the ORFs having the nucleotide sequence represented by SEQ ID NO: 174307.

The probes having the nucleotide sequence represented by SEQ ID NOS: 187561, 187562, 187563, 187564, 187565, 187566, 187567, 187568, 187569, 187570, 187571 were selected from the ORFs having the nucleotide sequence represented by SEQ ID NO: 174308.

The probes having the nucleotide sequence represented by SEQ ID NOS: 187572, 187573, 187574, 187575, 187576, 187577, 187578, 187579, 187580, 187581, 187582 were selected from the ORFs having the nucleotide sequence represented by SEQ ID NO: 174309.

The probes having the nucleotide sequence represented by SEQ ID NOS: 187583, 187584, 187585, 187586, 187587, 187588, 187589, 187590, 187591, 187592, 187593 were selected from the ORFs having the nucleotide sequence represented by SEQ ID NO: 174310.

The probes having the nucleotide sequence represented by SEQ ID NOS: 187594, 187595, 187596, 187597, 187598, 187599, 187600, 187601, 187602, 187603, 187604 were selected from the ORFs having the nucleotide sequence represented by SEQ ID NO: 174311.

The probes having the nucleotide sequence represented by SEQ ID NOS: 187605, 187606, 187607, 187608, 187609, 187610, 187611, 187612, 187613, 187614, 187615 were selected from the ORFs having the nucleotide sequence represented by SEQ ID NO: 174312.

The probes having the nucleotide sequence represented by SEQ ID NOS: 187616, 187617, 187618, 187619, 187620, 187621, 187622, 187623, 187624, 187625, 187626 were selected from the ORFs having the nucleotide sequence represented by SEQ ID NO: 174313.

The probes having the nucleotide sequence represented by SEQ ID NOS: 187627, 187628, 187629, 187630, 187631, 187632, 187633, 187634, 187635, 187636, 187637 were selected from the ORFs having the nucleotide sequence represented by SEQ ID NO: 174314.

The probes having the nucleotide sequence represented by SEQ ID NOS: 187638, 187639, 187640, 187641, 187642, 187643, 187644, 187645, 187646, 187647, 187648 were selected from the ORFs having the nucleotide sequence represented by SEQ ID NO: 174315.

The probes having the nucleotide sequence represented by SEQ ID NOS: 187649, 187650, 187651, 187652, 187653, 187654, 187655, 187656, 187657, 187658, 187659 were selected from the ORFs having the nucleotide sequence represented by SEQ ID NO: 174316.

The probes having the nucleotide sequence represented by SEQ ID NOS: 187660, 187661, 187662, 187663, 187664, 187665, 187666, 187667, 187668, 187669, 187670 were selected from the ORFs having the nucleotide sequence represented by SEQ ID NO: 174317.

The probes having the nucleotide sequence represented by SEQ ID NOS: 187671, 187672, 187673, 187674, 187675, 187676, 187677, 187678, 187679, 187680, 187681 were selected from the ORFs having the nucleotide sequence represented by SEQ ID NO: 174318.

The probes having the nucleotide sequence represented by SEQ ID NOS: 187682, 187683, 187684, 187685, 187686, 187687, 187688, 187689, 187690, 187691, 187692 were selected from the ORFs having the nucleotide sequence represented by SEQ ID NO: 174319.

The probes having the nucleotide sequence represented by SEQ ID NOS: 187693, 187694, 187695, 187696, 187697, 187698, 187699, 187700, 187701, 187702, 187703 were selected from the ORFs having the nucleotide sequence represented by SEQ ID NO: 174320.

The probes having the nucleotide sequence represented by SEQ ID NOS: 187704, 187705, 187706, 187707, 187708, 187709, 187710, 187711, 187712, 187713, 187714 were selected from the ORFs having the nucleotide sequence represented by SEQ ID NO: 174321.

The probes having the nucleotide sequence represented by SEQ ID NOS: 187715, 187716, 187717, 187718, 187719, 187720, 187721, 187722, 187723, 187724, 187725 were selected from the ORFs having the nucleotide sequence represented by SEQ ID NO: 174322.

The probes having the nucleotide sequence represented by SEQ ID NOS: 187726, 187727, 187728, 187729, 187730, 187731, 187732, 187733, 187734, 187735, 187736 were selected from the ORFs having the nucleotide sequence represented by SEQ ID NO: 174323.

The probes having the nucleotide sequence represented by SEQ ID NOS: 187737, 187738, 187739, 187740, 187741, 187742, 187743, 187744, 187745, 187746, 187747 were selected from the ORFs having the nucleotide sequence represented by SEQ ID NO: 174324.

The probes having the nucleotide sequence represented by SEQ ID NOS: 187748, 187749, 187750, 187751, 187752, 187753, 187754, 187755, 187756, 187757, 187758 were selected from the ORFs having the nucleotide sequence represented by SEQ ID NO: 174325.

The probes having the nucleotide sequence represented by SEQ ID NOS: 187759, 187760, 187761, 187762, 187763, 187764, 187765, 187766, 187767, 187768, 187769 were selected from the ORFs having the nucleotide sequence represented by SEQ ID NO: 174326.

The probes having the nucleotide sequence represented by SEQ ID NOS: 187770, 187771, 187772, 187773, 187774, 187775, 187776, 187777, 187778, 187779, 187780 were selected from the ORFs having the nucleotide sequence represented by SEQ ID NO: 174327.

The probes having the nucleotide sequence represented by SEQ ID NOS: 187781, 187782, 187783, 187784, 187785, 187786, 187787, 187788, 187789, 187790, 187791 were selected from the ORFs having the nucleotide sequence represented by SEQ ID NO: 174328.

The probes having the nucleotide sequence represented by SEQ ID NOS: 187792, 187793, 187794, 187795, 187796, 187797, 187798, 187799, 187800, 187801 were selected from the ORFs having the nucleotide sequence represented by SEQ ID NO: 174329.

The probes having the nucleotide sequence represented by SEQ ID NOS: 187802, 187803, 187804, 187805, 187806, 187807, 187808, 187809, 187810, 187811, 187812 were selected from the ORFs having the nucleotide sequence represented by SEQ ID NO: 174330.

The probes having the nucleotide sequence represented by SEQ ID NOS: 187813, 187814, 187815, 187816, 187817, 187818, 187819, 187820, 187821, 187822, 187823 were selected from the ORFs having the nucleotide sequence represented by SEQ ID NO: 174331.

The probes having the nucleotide sequence represented by SEQ ID NOS: 187824, 187825, 187826, 187827, 187828, 187829, 187830, 187831, 187832, 187833, 187834 were selected from the ORFs having the nucleotide sequence represented by SEQ ID NO: 174332.

The probes having the nucleotide sequence represented by SEQ ID NOS: 187835, 187836, 187837, 187838, 187839, 187840, 187841, 187842, 187843, 187844, 187845 were selected from the ORFs having the nucleotide sequence represented by SEQ ID NO: 174333.

The probes having the nucleotide sequence represented by SEQ ID NOS: 187846, 187847, 187848, 187849, 187850, 187851, 187852, 187853, 187854, 187855, 187856 were selected from the ORFs having the nucleotide sequence represented by SEQ ID NO: 174334.

The probes having the nucleotide sequence represented by SEQ ID NOS: 187857, 187858, 187859, 187860, 187861, 187862, 187863, 187864, 187865, 187866, 187867 were selected from the ORFs having the nucleotide sequence represented by SEQ ID NO: 174335.

The probes having the nucleotide sequence represented by SEQ ID NOS: 187868, 187869, 187870, 187871, 187872, 187873, 187874, 187875, 187876, 187877 were selected from the ORFs having the nucleotide sequence represented by SEQ ID NO: 174336.

The probes having the nucleotide sequence represented by SEQ ID NOS: 187878, 187879, 187880, 187881, 187882, 187883, 187884, 187885, 187886, 187887, 187888 were selected from the ORFs having the nucleotide sequence represented by SEQ ID NO: 174337.

The probes having the nucleotide sequence represented by SEQ ID NOS: 187889, 187890, 187891, 187892, 187893, 187894, 187895, 187896, 187897, 187898, 187899 were selected from the ORFs having the nucleotide sequence represented by SEQ ID NO: 174338.

The probes having the nucleotide sequence represented by SEQ ID NOS: 187900, 187901, 187902, 187903, 187904, 187905, 187906, 187907, 187908, 187909, 187910 were selected from the ORFs having the nucleotide sequence represented by SEQ ID NO: 174339.

The probes having the nucleotide sequence represented by SEQ ID NOS: 187911, 187912, 187913, 187914, 187915, 187916, 187917, 187918, 187919, 187920, 187921 were selected from the ORFs having the nucleotide sequence represented by SEQ ID NO: 174340.

The probes having the nucleotide sequence represented by SEQ ID NOS: 187922, 187923, 187924, 187925, 187926, 187927, 187928, 187929, 187930, 187931, 187932 were selected from the ORFs having the nucleotide sequence represented by SEQ ID NO: 174341.

The probes having the nucleotide sequence represented by SEQ ID NOS: 187933, 187934, 187935, 187936, 187937, 187938, 187939, 187940, 187941, 187942, 187943 were selected from the ORFs having the nucleotide sequence represented by SEQ ID NO: 174342.

The probes having the nucleotide sequence represented by SEQ ID NOS: 187944, 187945, 187946, 187947, 187948, 187949, 187950, 187951, 187952, 187953, 187954 were selected from the ORFs having the nucleotide sequence represented by SEQ ID NO: 174343.

The probes having the nucleotide sequence represented by SEQ ID NOS: 187955, 187956, 187957, 187958, 187959, 187960, 187961, 187962, 187963, 187964, 187965 were selected from the ORFs having the nucleotide sequence represented by SEQ ID NO: 174344.

The probes having the nucleotide sequence represented by SEQ ID NOS: 187966, 187967, 187968, 187969, 187970, 187971, 187972, 187973, 187974, 187975, 187976 were selected from the ORFs having the nucleotide sequence represented by SEQ ID NO: 174345.

The probes having the nucleotide sequence represented by SEQ ID NOS: 187977, 187978, 187979, 187980, 187981, 187982, 187983, 187984, 187985, 187986, 187987 were selected from the ORFs having the nucleotide sequence represented by SEQ ID NO: 174346.

The probes having the nucleotide sequence represented by SEQ ID NOS: 187988, 187989, 187990, 187991, 187992, 187993, 187994, 187995, 187996, 187997, 187998 were selected from the ORFs having the nucleotide sequence represented by SEQ ID NO: 174347.

The probes having the nucleotide sequence represented by SEQ ID NOS: 187999, 188000, 188001, 188002, 188003, 188004, 188005, 188006, 188007, 188008, 188009 were selected from the ORFs having the nucleotide sequence represented by SEQ ID NO: 174348.

The probes having the nucleotide sequence represented by SEQ ID NOS: 188010, 188011, 188012, 188013, 188014, 188015, 188016, 188017, 188018, 188019, 188020 were selected from the ORFs having the nucleotide sequence represented by SEQ ID NO: 174349.

The probes having the nucleotide sequence represented by SEQ ID NOS: 188021, 188022, 188023, 188024, 188025, 188026, 188027, 188028, 188029, 188030, 188031 were selected from the ORFs having the nucleotide sequence represented by SEQ ID NO: 174350.

The probes having the nucleotide sequence represented by SEQ ID NOS: 188032, 188033, 188034, 188035, 188036, 188037, 188038, 188039, 188040, 188041, 188042 were selected from the ORFs having the nucleotide sequence represented by SEQ ID NO: 174351.

The probes having the nucleotide sequence represented by SEQ ID NOS: 188043, 188044, 188045, 188046, 188047, 188048, 188049, 188050, 188051, 188052, 188053 were selected from the ORFs having the nucleotide sequence represented by SEQ ID NO: 174352.

The probes having the nucleotide sequence represented by SEQ ID NOS: 188054, 188055, 188056, 188057, 188058, 188059, 188060, 188061, 188062, 188063, 188064 were selected from the ORFs having the nucleotide sequence represented by SEQ ID NO: 174353.

The probes having the nucleotide sequence represented by SEQ ID NOS: 188065, 188066, 188067, 188068, 188069, 188070, 188071, 188072, 188073, 188074, 188075 were selected from the ORFs having the nucleotide sequence represented by SEQ ID NO: 174354.

The probes having the nucleotide sequence represented by SEQ ID NOS: 188076, 188077, 188078, 188079, 188080, 188081, 188082, 188083, 188084, 188085, 188086 were selected from the ORFs having the nucleotide sequence represented by SEQ ID NO: 174355.

The probes having the nucleotide sequence represented by SEQ ID NOS: 188087, 188088, 188089, 188090, 188091, 188092, 188093, 188094, 188095, 188096, 188097 were selected from the ORFs having the nucleotide sequence represented by SEQ ID NO: 174356.

The probes having the nucleotide sequence represented by SEQ ID NOS: 188098, 188099, 188100, 188101, 188102, 188103, 188104, 188105, 188106, 188107, 188108 were selected from the ORFs having the nucleotide sequence represented by SEQ ID NO: 174357.

The probes having the nucleotide sequence represented by SEQ ID NOS: 188109, 188110, 188111, 188112, 188113, 188114, 188115, 188116, 188117, 188118, 188119 were selected from the ORFs having the nucleotide sequence represented by SEQ ID NO: 174358.

The probes having the nucleotide sequence represented by SEQ ID NOS: 188120, 188121, 188122, 188123, 188124, 188125, 188126, 188127, 188128, 188129, 188130 were selected from the ORFs having the nucleotide sequence represented by SEQ ID NO: 174359.

The probes having the nucleotide sequence represented by SEQ ID NOS: 188131, 188132, 188133, 188134, 188135, 188136, 188137, 188138, 188139, 188140, 188141 were selected from the ORFs having the nucleotide sequence represented by SEQ ID NO: 174360.

The probes having the nucleotide sequence represented by SEQ ID NOS: 188142, 188143, 188144, 188145, 188146, 188147, 188148, 188149, 188150, 188151, 188152 were selected from the ORFs having the nucleotide sequence represented by SEQ ID NO: 174361.

The probes having the nucleotide sequence represented by SEQ ID NOS: 188153, 188154, 188155, 188156, 188157, 188158, 188159, 188160, 188161, 188162, 188163 were selected from the ORFs having the nucleotide sequence represented by SEQ ID NO: 174362.

The probes having the nucleotide sequence represented by SEQ ID NOS: 188164, 188165, 188166, 188167, 188168, 188169, 188170, 188171, 188172, 188173, 188174 were selected from the ORFs having the nucleotide sequence represented by SEQ ID NO: 174363.

The probes having the nucleotide sequence represented by SEQ ID NOS: 188175, 188176, 188177, 188178, 188179, 188180, 188181, 188182, 188183, 188184, 188185 were selected from the ORFs having the nucleotide sequence represented by SEQ ID NO: 174364.

The probes having the nucleotide sequence represented by SEQ ID NOS: 188186, 188187, 188188, 188189, 188190, 188191, 188192, 188193, 188194, 188195, 188196 were selected from the ORFs having the nucleotide sequence represented by SEQ ID NO: 174365.

The probes having the nucleotide sequence represented by SEQ ID NOS: 188197, 188198, 188199, 188200, 188201, 188202, 188203, 188204, 188205, 188206, 188207 were selected from the ORFs having the nucleotide sequence represented by SEQ ID NO: 174366.

The probes having the nucleotide sequence represented by SEQ ID NOS: 188208, 188209, 188210, 188211, 188212, 188213, 188214, 188215, 188216, 188217, 188218 were selected from the ORFs having the nucleotide sequence represented by SEQ ID NO: 174367.

The probes having the nucleotide sequence represented by SEQ ID NOS: 188219, 188220, 188221, 188222, 188223, 188224, 188225, 188226, 188227, 188228, 188229 were selected from the ORFs having the nucleotide sequence represented by SEQ ID NO: 174368.

The probes having the nucleotide sequence represented by SEQ ID NOS: 188230, 188231, 188232, 188233, 188234, 188235, 188236, 188237, 188238, 188239, 188240 were selected from the ORFs having the nucleotide sequence represented by SEQ ID NO: 174369.

The probes having the nucleotide sequence represented by SEQ ID NOS: 188241, 188242, 188243, 188244, 188245, 188246, 188247, 188248, 188249, 188250, 188251 were selected from the ORFs having the nucleotide sequence represented by SEQ ID NO: 174370.

The probes having the nucleotide sequence represented by SEQ ID NOS: 188252, 188253, 188254, 188255, 188256, 188257, 188258, 188259, 188260, 188261, 188262 were selected from the ORFs having the nucleotide sequence represented by SEQ ID NO: 174371.

The probes having the nucleotide sequence represented by SEQ ID NOS: 188263, 188264, 188265, 188266, 188267, 188268, 188269, 188270, 188271, 188272, 188273 were selected from the ORFs having the nucleotide sequence represented by SEQ ID NO: 174372.

The probes having the nucleotide sequence represented by SEQ ID NOS: 188274, 188275, 188276, 188277, 188278, 188279, 188280, 188281, 188282, 188283, 188284 were selected from the ORFs having the nucleotide sequence represented by SEQ ID NO: 174373.

The probes having the nucleotide sequence represented by SEQ ID NOS: 188285, 188286, 188287, 188288, 188289, 188290, 188291, 188292, 188293, 188294, 188295 were selected from the ORFs having the nucleotide sequence represented by SEQ ID NO: 174374.

The probes having the nucleotide sequence represented by SEQ ID NOS: 188296, 188297, 188298, 188299, 188300, 188301, 188302, 188303, 188304, 188305, 188306 were selected from the ORFs having the nucleotide sequence represented by SEQ ID NO: 174375.

The probes having the nucleotide sequence represented by SEQ ID NOS: 188307, 188308, 188309, 188310, 188311, 188312, 188313, 188314, 188315, 188316, 188317 were selected from the ORFs having the nucleotide sequence represented by SEQ ID NO: 174376.

The probes having the nucleotide sequence represented by SEQ ID NOS: 188318, 188319, 188320, 188321, 188322, 188323, 188324, 188325, 188326, 188327, 188328 were selected from the ORFs having the nucleotide sequence represented by SEQ ID NO: 174377.

The probes having the nucleotide sequence represented by SEQ ID NOS: 188329, 188330, 188331, 188332, 188333, 188334, 188335, 188336, 188337, 188338, 188339 were selected from the ORFs having the nucleotide sequence represented by SEQ ID NO: 174378.

The probes having the nucleotide sequence represented by SEQ ID NOS: 188340, 188341, 188342, 188343, 188344, 188345, 188346, 188347, 188348, 188349, 188350 were selected from the ORFs having the nucleotide sequence represented by SEQ ID NO: 174379.

The probes having the nucleotide sequence represented by SEQ ID NOS: 188351, 188352, 188353, 188354, 188355, 188356, 188357, 188358, 188359, 188360, 188361 were selected from the ORFs having the nucleotide sequence represented by SEQ ID NO: 174380.

The probes having the nucleotide sequence represented by SEQ ID NOS: 188362, 188363, 188364, 188365, 188366, 188367, 188368, 188369 were selected from the ORFs having the nucleotide sequence represented by SEQ ID NO: 174381.

The probes having the nucleotide sequence represented by SEQ ID NOS: 188370, 188371, 188372, 188373, 188374, 188375, 188376, 188377, 188378, 188379, 188380 were selected from the ORFs having the nucleotide sequence represented by SEQ ID NO: 174382.

The probes having the nucleotide sequence represented by SEQ ID NOS: 188381, 188382, 188383, 188384, 188385, 188386, 188387, 188388, 188389, 188390, 188391 were selected from the ORFs having the nucleotide sequence represented by SEQ ID NO: 174383.

The probes having the nucleotide sequence represented by SEQ ID NOS: 188392, 188393, 188394, 188395, 188396, 188397, 188398, 188399, 188400, 188401, 188402 were selected from the ORFs having the nucleotide sequence represented by SEQ ID NO: 174384.

The probes having the nucleotide sequence represented by SEQ ID NOS: 188403, 188404, 188405, 188406, 188407, 188408, 188409, 188410, 188411, 188412, 188413 were selected from the ORFs having the nucleotide sequence represented by SEQ ID NO: 174385.

The probes having the nucleotide sequence represented by SEQ ID NOS: 188414, 188415, 188416, 188417, 188418, 188419, 188420, 188421, 188422, 188423, 188424 were selected from the ORFs having the nucleotide sequence represented by SEQ ID NO: 174386.

The probes having the nucleotide sequence represented by SEQ ID NOS: 188425, 188426, 188427, 188428, 188429, 188430, 188431, 188432, 188433, 188434, 188435 were selected from the ORFs having the nucleotide sequence represented by SEQ ID NO: 174387.

The probes having the nucleotide sequence represented by SEQ ID NOS: 188436, 188437, 188438, 188439, 188440, 188441, 188442, 188443, 188444, 188445, 188446 were selected from the ORFs having the nucleotide sequence represented by SEQ ID NO: 174388.

The probes having the nucleotide sequence represented by SEQ ID NOS: 188447, 188448, 188449, 188450, 188451, 188452, 188453, 188454, 188455, 188456, 188457 were selected from the ORFs having the nucleotide sequence represented by SEQ ID NO: 174389.

The probes having the nucleotide sequence represented by SEQ ID NOS: 188458, 188459, 188460, 188461, 188462, 188463, 188464, 188465, 188466, 188467, 188468 were selected from the ORFs having the nucleotide sequence represented by SEQ ID NO: 174390.

The probes having the nucleotide sequence represented by SEQ ID NOS: 188469, 188470, 188471, 188472, 188473, 188474, 188475, 188476, 188477, 188478, 188479 were selected from the ORFs having the nucleotide sequence represented by SEQ ID NO: 174391.

The probes having the nucleotide sequence represented by SEQ ID NOS: 188480, 188481, 188482, 188483, 188484, 188485, 188486, 188487, 188488, 188489, 188490 were selected from the ORFs having the nucleotide sequence represented by SEQ ID NO: 174392.

The probes having the nucleotide sequence represented by SEQ ID NOS: 188491, 188492, 188493, 188494, 188495, 188496, 188497, 188498, 188499, 188500, 188501 were selected from the ORFs having the nucleotide sequence represented by SEQ ID NO: 174393.

The probes having the nucleotide sequence represented by SEQ ID NOS: 188502, 188503, 188504, 188505, 188506, 188507, 188508, 188509, 188510, 188511, 188512 were selected from the ORFs having the nucleotide sequence represented by SEQ ID NO: 174394.

The probes having the nucleotide sequence represented by SEQ ID NOS: 188513, 188514, 188515, 188516, 188517, 188518, 188519, 188520, 188521, 188522, 188523 were selected from the ORFs having the nucleotide sequence represented by SEQ ID NO: 174395.

The probes having the nucleotide sequence represented by SEQ ID NOS: 188524, 188525, 188526, 188527, 188528, 188529, 188530, 188531, 188532, 188533, 188534 were selected from the ORFs having the nucleotide sequence represented by SEQ ID NO: 174396.

The probes having the nucleotide sequence represented by SEQ ID NOS: 188535, 188536, 188537, 188538, 188539, 188540, 188541, 188542, 188543, 188544, 188545 were selected from the ORFs having the nucleotide sequence represented by SEQ ID NO: 174397.

The probes having the nucleotide sequence represented by SEQ ID NOS: 188546, 188547, 188548, 188549, 188550, 188551, 188552, 188553, 188554, 188555, 188556 were selected from the ORFs having the nucleotide sequence represented by SEQ ID NO: 174398.

The probes having the nucleotide sequence represented by SEQ ID NOS: 188557, 188558, 188559, 188560, 188561, 188562, 188563, 188564, 188565, 188566, 188567 were selected from the ORFs having the nucleotide sequence represented by SEQ ID NO: 174399.

The probes having the nucleotide sequence represented by SEQ ID NOS: 188568, 188569, 188570, 188571, 188572, 188573, 188574, 188575, 188576, 188577, 188578 were selected from the ORFs having the nucleotide sequence represented by SEQ ID NO: 174400.

The probes having the nucleotide sequence represented by SEQ ID NOS: 188579, 188580, 188581, 188582, 188583, 188584, 188585, 188586, 188587, 188588, 188589 were selected from the ORFs having the nucleotide sequence represented by SEQ ID NO: 174401.

The probes having the nucleotide sequence represented by SEQ ID NOS: 188590, 188591, 188592, 188593, 188594, 188595, 188596, 188597, 188598, 188599, 188600 were selected from the ORFs having the nucleotide sequence represented by SEQ ID NO: 174402.

The probes having the nucleotide sequence represented by SEQ ID NOS: 188601, 188602, 188603, 188604, 188605, 188606, 188607, 188608, 188609, 188610, 188611 were selected from the ORFs having the nucleotide sequence represented by SEQ ID NO: 174403.

The probes having the nucleotide sequence represented by SEQ ID NOS: 188612, 188613, 188614, 188615, 188616, 188617, 188618, 188619, 188620, 188621, 188622 were selected from the ORFs having the nucleotide sequence represented by SEQ ID NO: 174404.

The probes having the nucleotide sequence represented by SEQ ID NOS: 188623, 188624, 188625, 188626, 188627, 188628, 188629, 188630, 188631, 188632, 188633 were selected from the ORFs having the nucleotide sequence represented by SEQ ID NO: 174405.

The probes having the nucleotide sequence represented by SEQ ID NOS: 188634, 188635, 188636, 188637, 188638, 188639, 188640, 188641, 188642, 188643, 188644 were selected from the ORFs having the nucleotide sequence represented by SEQ ID NO: 174406.

The probes having the nucleotide sequence represented by SEQ ID NOS: 188645, 188646, 188647, 188648, 188649, 188650, 188651, 188652, 188653, 188654, 188655 were selected from the ORFs having the nucleotide sequence represented by SEQ ID NO: 174407.

The probes having the nucleotide sequence represented by SEQ ID NOS: 188656, 188657, 188658, 188659, 188660, 188661, 188662, 188663, 188664, 188665, 188666 were selected from the ORFs having the nucleotide sequence represented by SEQ ID NO: 174408.

The probes having the nucleotide sequence represented by SEQ ID NOS: 188667, 188668, 188669, 188670, 188671, 188672, 188673, 188674, 188675, 188676, 188677 were selected from the ORFs having the nucleotide sequence represented by SEQ ID NO: 174409.

The probes having the nucleotide sequence represented by SEQ ID NOS: 188678, 188679, 188680, 188681, 188682, 188683, 188684, 188685, 188686, 188687, 188688 were selected from the ORFs having the nucleotide sequence represented by SEQ ID NO: 174410.

The probes having the nucleotide sequence represented by SEQ ID NOS: 188689, 188690, 188691, 188692, 188693, 188694, 188695, 188696, 188697, 188698, 188699 were selected from the ORFs having the nucleotide sequence represented by SEQ ID NO: 174411.

The probes having the nucleotide sequence represented by SEQ ID NOS: 188700, 188701, 188702, 188703, 188704, 188705, 188706, 188707, 188708, 188709, 188710 were selected from the ORFs having the nucleotide sequence represented by SEQ ID NO: 174412.

The probes having the nucleotide sequence represented by SEQ ID NOS: 188711, 188712, 188713, 188714, 188715, 188716, 188717, 188718, 188719, 188720, 188721 were selected from the ORFs having the nucleotide sequence represented by SEQ ID NO: 174413.

The probes having the nucleotide sequence represented by SEQ ID NOS: 188722, 188723, 188724, 188725, 188726, 188727, 188728, 188729, 188730, 188731, 188732 were selected from the ORFs having the nucleotide sequence represented by SEQ ID NO: 174414.

The probes having the nucleotide sequence represented by SEQ ID NOS: 188733, 188734, 188735, 188736, 188737, 188738, 188739, 188740, 188741, 188742, 188743 were selected from the ORFs having the nucleotide sequence represented by SEQ ID NO: 174415.

The probes having the nucleotide sequence represented by SEQ ID NOS: 188744, 188745, 188746, 188747, 188748, 188749, 188750, 188751, 188752, 188753, 188754 were selected from the ORFs having the nucleotide sequence represented by SEQ ID NO: 174416.

The probes having the nucleotide sequence represented by SEQ ID NOS: 188755, 188756, 188757, 188758, 188759, 188760, 188761, 188762, 188763, 188764, 188765 were selected from the ORFs having the nucleotide sequence represented by SEQ ID NO: 174417.

The probes having the nucleotide sequence represented by SEQ ID NOS: 188766, 188767, 188768, 188769, 188770, 188771, 188772, 188773, 188774, 188775, 188776 were selected from the ORFs having the nucleotide sequence represented by SEQ ID NO: 174418.

The probes having the nucleotide sequence represented by SEQ ID NOS: 188777, 188778, 188779, 188780, 188781, 188782, 188783, 188784, 188785, 188786, 188787 were selected from the ORFs having the nucleotide sequence represented by SEQ ID NO: 174419.

The probes having the nucleotide sequence represented by SEQ ID NOS: 188788, 188789, 188790, 188791, 188792, 188793, 188794, 188795, 188796, 188797, 188798 were selected from the ORFs having the nucleotide sequence represented by SEQ ID NO: 174420.

The probes having the nucleotide sequence represented by SEQ ID NOS: 188799, 188800, 188801, 188802, 188803, 188804, 188805, 188806, 188807, 188808, 188809 were selected from the ORFs having the nucleotide sequence represented by SEQ ID NO: 174421.

The probes having the nucleotide sequence represented by SEQ ID NOS: 188810, 188811, 188812, 188813, 188814, 188815, 188816, 188817, 188818, 188819, 188820 were selected from the ORFs having the nucleotide sequence represented by SEQ ID NO: 174422.

The probes having the nucleotide sequence represented by SEQ ID NOS: 188821, 188822, 188823, 188824, 188825, 188826, 188827, 188828, 188829, 188830, 188831 were selected from the ORFs having the nucleotide sequence represented by SEQ ID NO: 174423.

The probes having the nucleotide sequence represented by SEQ ID NOS: 188832, 188833, 188834, 188835, 188836, 188837, 188838, 188839, 188840, 188841, 188842 were selected from the ORFs having the nucleotide sequence represented by SEQ ID NO: 174424.

The probes having the nucleotide sequence represented by SEQ ID NOS: 188843, 188844, 188845, 188846, 188847, 188848, 188849, 188850, 188851, 188852, 188853 were selected from the ORFs having the nucleotide sequence represented by SEQ ID NO: 174425.

The probes having the nucleotide sequence represented by SEQ ID NOS: 188854, 188855, 188856, 188857, 188858, 188859, 188860, 188861, 188862, 188863, 188864 were selected from the ORFs having the nucleotide sequence represented by SEQ ID NO: 174426.

The probes having the nucleotide sequence represented by SEQ ID NOS: 188865, 188866, 188867, 188868, 188869, 188870, 188871, 188872, 188873, 188874, 188875 were selected from the ORFs having the nucleotide sequence represented by SEQ ID NO: 174427.

The probes having the nucleotide sequence represented by SEQ ID NOS: 188876, 188877, 188878, 188879, 188880, 188881, 188882, 188883, 188884, 188885, 188886 were selected from the ORFs having the nucleotide sequence represented by SEQ ID NO: 174428.

The probes having the nucleotide sequence represented by SEQ ID NOS: 188887, 188888, 188889, 188890, 188891, 188892, 188893, 188894, 188895, 188896, 188897 were selected from the ORFs having the nucleotide sequence represented by SEQ ID NO: 174429.

The probes having the nucleotide sequence represented by SEQ ID NOS: 188898, 188899, 188900, 188901, 188902, 188903, 188904, 188905, 188906, 188907, 188908 were selected from the ORFs having the nucleotide sequence represented by SEQ ID NO: 174430.

The probes having the nucleotide sequence represented by SEQ ID NOS: 188909, 188910, 188911, 188912, 188913, 188914, 188915, 188916, 188917, 188918, 188919 were selected from the ORFs having the nucleotide sequence represented by SEQ ID NO: 174431.

The probes having the nucleotide sequence represented by SEQ ID NOS: 188920, 188921, 188922, 188923, 188924, 188925, 188926, 188927, 188928, 188929, 188930 were selected from the ORFs having the nucleotide sequence represented by SEQ ID NO: 174432.

The probes having the nucleotide sequence represented by SEQ ID NOS: 188931, 188932, 188933, 188934, 188935, 188936, 188937, 188938, 188939, 188940, 188941 were selected from the ORFs having the nucleotide sequence represented by SEQ ID NO: 174433.

The probes having the nucleotide sequence represented by SEQ ID NOS: 188942, 188943, 188944, 188945, 188946, 188947, 188948, 188949, 188950, 188951, 188952 were selected from the ORFs having the nucleotide sequence represented by SEQ ID NO: 174434.

The probes having the nucleotide sequence represented by SEQ ID NOS: 188953, 188954, 188955, 188956, 188957, 188958, 188959, 188960, 188961, 188962, 188963 were selected from the ORFs having the nucleotide sequence represented by SEQ ID NO: 174435.

The probes having the nucleotide sequence represented by SEQ ID NOS: 188964, 188965, 188966, 188967, 188968, 188969, 188970, 188971, 188972, 188973, 188974 were selected from the ORFs having the nucleotide sequence represented by SEQ ID NO: 174436.

The probes having the nucleotide sequence represented by SEQ ID NOS: 188975, 188976, 188977, 188978, 188979, 188980, 188981, 188982, 188983, 188984, 188985 were selected from the ORFs having the nucleotide sequence represented by SEQ ID NO: 174437.

The probes having the nucleotide sequence represented by SEQ ID NOS: 188986, 188987, 188988, 188989, 188990, 188991, 188992, 188993, 188994, 188995, 188996 were selected from the ORFs having the nucleotide sequence represented by SEQ ID NO: 174438.

The probes having the nucleotide sequence represented by SEQ ID NOS: 188997, 188998, 188999, 189000, 189001, 189002, 189003, 189004, 189005, 189006, 189007 were selected from the ORFs having the nucleotide sequence represented by SEQ ID NO: 174439.

The probes having the nucleotide sequence represented by SEQ ID NOS: 189008, 189009, 189010, 189011, 189012, 189013, 189014, 189015, 189016, 189017, 189018 were selected from the ORFs having the nucleotide sequence represented by SEQ ID NO: 174440.

The probes having the nucleotide sequence represented by SEQ ID NOS: 189019, 189020, 189021, 189022, 189023, 189024, 189025, 189026, 189027, 189028, 189029 were selected from the ORFs having the nucleotide sequence represented by SEQ ID NO: 174441.

The probes having the nucleotide sequence represented by SEQ ID NOS: 189030, 189031, 189032, 189033, 189034, 189035, 189036, 189037, 189038, 189039, 189040 were selected from the ORFs having the nucleotide sequence represented by SEQ ID NO: 174442.

The probes having the nucleotide sequence represented by SEQ ID NOS: 189041, 189042, 189043, 189044, 189045, 189046, 189047, 189048, 189049, 189050, 189051 were selected from the ORFs having the nucleotide sequence represented by SEQ ID NO: 174443.

The probes having the nucleotide sequence represented by SEQ ID NOS: 189052, 189053, 189054, 189055, 189056, 189057, 189058, 189059, 189060, 189061, 189062 were selected from the ORFs having the nucleotide sequence represented by SEQ ID NO: 174444.

The probes having the nucleotide sequence represented by SEQ ID NOS: 189063, 189064, 189065, 189066, 189067, 189068, 189069, 189070, 189071, 189072, 189073 were selected from the ORFs having the nucleotide sequence represented by SEQ ID NO: 174445.

The probes having the nucleotide sequence represented by SEQ ID NOS: 189074, 189075, 189076, 189077, 189078, 189079, 189080, 189081, 189082, 189083, 189084 were selected from the ORFs having the nucleotide sequence represented by SEQ ID NO: 174446.

The probes having the nucleotide sequence represented by SEQ ID NOS: 189085, 189086, 189087, 189088, 189089, 189090, 189091, 189092, 189093, 189094, 189095 were selected from the ORFs having the nucleotide sequence represented by SEQ ID NO: 174447.

The probes having the nucleotide sequence represented by SEQ ID NOS: 189096, 189097, 189098, 189099, 189100, 189101, 189102, 189103, 189104, 189105, 189106 were selected from the ORFs having the nucleotide sequence represented by SEQ ID NO: 174448.

The probes having the nucleotide sequence represented by SEQ ID NOS: 189107, 189108, 189109, 189110, 189111, 189112, 189113, 189114, 189115, 189116, 189117 were selected from the ORFs having the nucleotide sequence represented by SEQ ID NO: 174449.

The probes having the nucleotide sequence represented by SEQ ID NOS: 189118, 189119, 189120, 189121, 189122, 189123, 189124, 189125, 189126, 189127, 189128 were selected from the ORFs having the nucleotide sequence represented by SEQ ID NO: 174450.

The probes having the nucleotide sequence represented by SEQ ID NOS: 189129, 189130, 189131, 189132, 189133, 189134, 189135, 189136, 189137, 189138, 189139 were selected from the ORFs having the nucleotide sequence represented by SEQ ID NO: 174451.

The probes having the nucleotide sequence represented by SEQ ID NOS:. 189140, 189141, 189142, 189143, 189144, 189145, 189146, 189147, 189148, 189149, 189150 were selected from the ORFs having the nucleotide sequence represented by SEQ ID NO: 174452.

The probes having the nucleotide sequence represented by SEQ ID NOS: 189151, 189152, 189153, 189154, 189155, 189156, 189157, 189158, 189159, 189160, 189161 were selected from the ORFs having the nucleotide sequence represented by SEQ ID NO: 174453.

The probes having the nucleotide sequence represented by SEQ ID NOS: 189162, 189163, 189164, 189165, 189166, 189167, 189168, 189169, 189170, 189171, 189172 were selected from the ORFs having the nucleotide sequence represented by SEQ ID NO: 174454.

The probes having the nucleotide sequence represented by SEQ ID NOS: 189173, 189174, 189175, 189176, 189177, 189178, 189179, 189180, 189181, 189182, 189183 were selected from the ORFs having the nucleotide sequence represented by SEQ ID NO: 174455.

The probes having the nucleotide sequence represented by SEQ ID NOS: 189184, 189185, 189186, 189187, 189188, 189189, 189190, 189191, 189192, 189193, 189194 were selected from the ORFs having the nucleotide sequence represented by SEQ ID NO: 174456.

The probes having the nucleotide sequence represented by SEQ ID NOS: 189195, 189196, 189197, 189198, 189199, 189200, 189201, 189202, 189203, 189204, 189205 were selected from the ORFs having the nucleotide sequence represented by SEQ ID NO: 174457.

The probes having the nucleotide sequence represented by SEQ ID NOS: 189206, 189207, 189208, 189209, 189210, 189211, 189212, 189213, 189214, 189215, 189216 were selected from the ORFs having the nucleotide sequence represented by SEQ ID NO: 174458.

The probes having the nucleotide sequence represented by SEQ ID NOS: 189217, 189218, 189219, 189220, 189221, 189222, 189223, 189224, 189225, 189226, 189227 were selected from the ORFs having the nucleotide sequence represented by SEQ ID NO: 174459.

The probes having the nucleotide sequence represented by SEQ ID NOS: 189228, 189229, 189230, 189231, 189232, 189233, 189234, 189235, 189236, 189237, 189238 were selected from the ORFs having the nucleotide sequence represented by SEQ ID NO: 174460.

The probes having the nucleotide sequence represented by SEQ ID NOS: 189239, 189240, 189241, 189242, 189243, 189244, 189245, 189246, 189247, 189248, 189249 were selected from the ORFs having the nucleotide sequence represented by SEQ ID NO: 174461.

The probes having the nucleotide sequence represented by SEQ ID NOS: 189250, 189251, 189252, 189253, 189254, 189255, 189256, 189257, 189258, 189259, 189260 were selected from the ORFs having the nucleotide sequence represented by SEQ ID NO: 174462.

The probes having the nucleotide sequence represented by SEQ ID NOS: 189261, 189262, 189263, 189264, 189265, 189266, 189267, 189268, 189269, 189270, 189271 were selected from the ORFs having the nucleotide sequence represented by SEQ ID NO: 174463.

The probes having the nucleotide sequence represented by SEQ ID NOS: 189272, 189273, 189274, 189275, 189276, 189277, 189278, 189279, 189280, 189281, 189282 were selected from the ORFs having the nucleotide sequence represented by SEQ ID NO: 174464.

The probes having the nucleotide sequence represented by SEQ ID NOS: 189283, 189284, 189285, 189286, 189287, 189288, 189289, 189290, 189291, 189292, 189293 were selected from the ORFs having the nucleotide sequence represented by SEQ ID NO: 174465.

The probes having the nucleotide sequence represented by SEQ ID NOS: 189294, 189295, 189296, 189297, 189298, 189299, 189300, 189301, 189302, 189303, 189304 were selected from the ORFs having the nucleotide sequence represented by SEQ ID NO: 174466.

The probes having the nucleotide sequence represented by SEQ ID NOS: 189305, 189306, 189307, 189308, 189309, 189310, 189311, 189312, 189313, 189314, 189315 were selected from the ORFs having the nucleotide sequence represented by SEQ ID NO: 174467.

The probes having the nucleotide sequence represented by SEQ ID NOS: 189316, 189317, 189318, 189319, 189320, 189321, 189322, 189323, 189324, 189325, 189326 were selected from the ORFs having the nucleotide sequence represented by SEQ ID NO: 174468.

The probes having the nucleotide sequence represented by SEQ ID NOS: 189327, 189328, 189329, 189330, 189331, 189332, 189333, 189334, 189335, 189336, 189337 were selected from the ORFs having the nucleotide sequence represented by SEQ ID NO: 174469.

The probes having the nucleotide sequence represented by SEQ ID NOS: 189338, 189339, 189340, 189341, 189342, 189343, 189344, 189345, 189346, 189347, 189348 were selected from the ORFs having the nucleotide sequence represented by SEQ ID NO: 174470.

The probes having the nucleotide sequence represented by SEQ ID NOS: 189349, 189350, 189351, 189352, 189353, 189354, 189355, 189356, 189357, 189358, 189359 were selected from the ORFs having the nucleotide sequence represented by SEQ ID NO: 174471.

The probes having the nucleotide sequence represented by SEQ ID NOS: 189360, 189361, 189362, 189363, 189364, 189365, 189366, 189367, 189368, 189369, 189370 were selected from the ORFs having the nucleotide sequence represented by SEQ ID NO: 174472.

The probes having the nucleotide sequence represented by SEQ ID NOS: 189371, 189372, 189373, 189374, 189375, 189376, 189377, 189378, 189379, 189380, 189381 were selected from the ORFs having the nucleotide sequence represented by SEQ ID NO: 174473.

The probes having the nucleotide sequence represented by SEQ ID NOS: 189382, 189383, 189384, 189385, 189386, 189387, 189388, 189389, 189390, 189391, 189392 were selected from the ORFs having the nucleotide sequence represented by SEQ ID NO: 174474.

The probes having the nucleotide sequence represented by SEQ ID NOS: 189393, 189394, 189395, 189396, 189397, 189398, 189399, 189400, 189401, 189402, 189403 were selected from the ORFs having the nucleotide sequence represented by SEQ ID NO: 174475.

The probes having the nucleotide sequence represented by SEQ ID NOS: 189404, 189405, 189406, 189407, 189408, 189409, 189410, 189411, 189412, 189413, 189414 were selected from the ORFs having the nucleotide sequence represented by SEQ ID NO: 174476.

The probes having the nucleotide sequence represented by SEQ ID NOS: 189415, 189416, 189417, 189418, 189419, 189420, 189421, 189422, 189423, 189424, 189425 were selected from the ORFs having the nucleotide sequence represented by SEQ ID NO: 174477.

The probes having the nucleotide sequence represented by SEQ ID NOS: 189426, 189427, 189428, 189429, 189430, 189431, 189432, 189433, 189434, 189435, 189436 were selected from the ORFs having the nucleotide sequence represented by SEQ ID NO: 174478.

The probes having the nucleotide sequence represented by SEQ ID NOS: 189437, 189438, 189439, 189440, 189441, 189442, 189443, 189444, 189445, 189446 were selected from the ORFs having the nucleotide sequence represented by SEQ ID NO: 174479.

The probes having the nucleotide sequence represented by SEQ ID NOS: 189447, 189448, 189449, 189450, 189451, 189452, 189453, 189454, 189455, 189456, 189457 were selected from the ORFs having the nucleotide sequence represented by SEQ ID NO: 174480.

The probes having the nucleotide sequence represented by SEQ ID NOS: 189458, 189459, 189460, 189461, 189462, 189463, 189464, 189465, 189466, 189467, 189468 were selected from the ORFs having the nucleotide sequence represented by SEQ ID NO: 174481.

The probes having the nucleotide sequence represented by SEQ ID NOS: 189469, 189470, 189471, 189472, 189473, 189474, 189475, 189476, 189477, 189478, 189479 were selected from the ORFs having the nucleotide sequence represented by SEQ ID NO: 174482.

The probes having the nucleotide sequence represented by SEQ ID NOS: 189480, 189481, 189482, 189483, 189484, 189485, 189486, 189487, 189488, 189489, 189490 were selected from the ORFs having the nucleotide sequence represented by SEQ ID NO: 174483.

The probes having the nucleotide sequence represented by SEQ ID NOS: 189491, 189492, 189493, 189494, 189495, 189496, 189497, 189498, 189499, 189500, 189501 were selected from the ORFs having the nucleotide sequence represented by SEQ ID NO: 174484.

The probes having the nucleotide sequence represented by SEQ ID NOS: 189502, 189503, 189504, 189505, 189506, 189507, 189508, 189509, 189510, 189511, 189512 were selected from the ORFs having the nucleotide sequence represented by SEQ ID NO: 174485.

The probes having the nucleotide sequence represented by SEQ ID NOS: 189513, 189514, 189515, 189516, 189517, 189518, 189519, 189520, 189521, 189522, 189523 were selected from the ORFs having the nucleotide sequence represented by SEQ ID NO: 174486.

The probes having the nucleotide sequence represented by SEQ ID NOS: 189524, 189525, 189526, 189527, 189528, 189529, 189530, 189531, 189532, 189533, 189534 were selected from the ORFs having the nucleotide sequence represented by SEQ ID NO: 174487.

The probes having the nucleotide sequence represented by SEQ ID NOS: 189535, 189536, 189537, 189538, 189539, 189540, 189541, 189542, 189543, 189544, 189545 were selected from the ORFs having the nucleotide sequence represented by SEQ ID NO: 174488.

The probes having the nucleotide sequence represented by SEQ ID NOS: 189546, 189547, 189548, 189549, 189550, 189551, 189552, 189553, 189554, 189555, 189556 were selected from the ORFs having the nucleotide sequence represented by SEQ ID NO: 174489.

The probes having the nucleotide sequence represented by SEQ ID NOS: 189557, 189558, 189559, 189560, 189561, 189562, 189563, 189564, 189565, 189566, 189567 were selected from the ORFs having the nucleotide sequence represented by SEQ ID NO: 174490.

The probes having the nucleotide sequence represented by SEQ ID NOS: 189568, 189569, 189570, 189571, 189572, 189573, 189574, 189575, 189576, 189577, 189578 were selected from the ORFs having the nucleotide sequence represented by SEQ ID NO: 174491.

The probes having the nucleotide sequence represented by SEQ ID NOS: 189579, 189580, 189581, 189582, 189583, 189584, 189585, 189586, 189587, 189588, 189589 were selected from the ORFs having the nucleotide sequence represented by SEQ ID NO: 174492.

The probes having the nucleotide sequence represented by SEQ ID NOS: 189590, 189591, 189592, 189593, 189594, 189595, 189596, 189597, 189598, 189599, 189600 were selected from the ORFs having the nucleotide sequence represented by SEQ ID NO: 174493.

The probes having the nucleotide sequence represented by SEQ ID NOS: 189601, 189602, 189603, 189604, 189605, 189606, 189607, 189608, 189609, 189610, 189611 were selected from the ORFs having the nucleotide sequence represented by SEQ ID NO: 174494.

The probes having the nucleotide sequence represented by SEQ ID NOS: 189612, 189613, 189614, 189615, 189616, 189617, 189618, 189619, 189620, 189621, 189622 were selected from the ORFs having the nucleotide sequence represented by SEQ ID NO: 174495.

The probes having the nucleotide sequence represented by SEQ ID NOS: 189623, 189624, 189625, 189626, 189627, 189628, 189629, 189630, 189631, 189632, 189633 were selected from the ORFs having the nucleotide sequence represented by SEQ ID NO: 174496.

The probes having the nucleotide sequence represented by SEQ ID NOS: 189634, 189635, 189636, 189637, 189638, 189639, 189640, 189641, 189642, 189643, 189644 were selected from the ORFs having the nucleotide sequence represented by SEQ ID NO: 174497.

The probes having the nucleotide sequence represented by SEQ ID NOS: 189645, 189646, 189647, 189648, 189649, 189650, 189651, 189652, 189653, 189654, 189655 were selected from the ORFs having the nucleotide sequence represented by SEQ ID NO: 174498.

The probes having the nucleotide sequence represented by SEQ ID NOS: 189656, 189657, 189658, 189659, 189660, 189661, 189662, 189663, 189664, 189665, 189666 were selected from the ORFs having the nucleotide sequence represented by SEQ ID NO: 174499.

The probes having the nucleotide sequence represented by SEQ ID NOS: 189667, 189668, 189669, 189670, 189671, 189672, 189673, 189674, 189675, 189676, 189677 were selected from the ORFs having the nucleotide sequence represented by SEQ ID NO: 174500.

The probes having the nucleotide sequence represented by SEQ ID NOS: 189678, 189679, 189680, 189681, 189682, 189683, 189684, 189685, 189686, 189687, 189688 were selected from the ORFs having the nucleotide sequence represented by SEQ ID NO: 174501.

The probes having the nucleotide sequence represented by SEQ ID NOS: 189689, 189690, 189691, 189692, 189693, 189694, 189695, 189696, 189697, 189698, 189699 were selected from the ORFs having the nucleotide sequence represented by SEQ ID NO: 174502.

The probes having the nucleotide sequence represented by SEQ ID NOS: 189700, 189701, 189702, 189703, 189704, 189705, 189706, 189707, 189708, 189709, 189710 were selected from the ORFs having the nucleotide sequence represented by SEQ ID NO: 174503.

The probes having the nucleotide sequence represented by SEQ ID NOS: 189711, 189712, 189713, 189714, 189715, 189716, 189717, 189718, 189719, 189720, 189721 were selected from the ORFs having the nucleotide sequence represented by SEQ ID NO: 174504.

The probes having the nucleotide sequence represented by SEQ ID NOS: 189722, 189723, 189724, 189725, 189726, 189727, 189728, 189729, 189730, 189731, 189732 were selected from the ORFs having the nucleotide sequence represented by SEQ ID NO: 174505.

The probes having the nucleotide sequence represented by SEQ ID NOS: 189733, 189734, 189735, 189736, 189737, 189738, 189739, 189740, 189741, 189742, 189743 were selected from the ORFs having the nucleotide sequence represented by SEQ ID NO: 174506.

The probes having the nucleotide sequence represented by SEQ ID NOS: 189744, 189745, 189746, 189747, 189748, 189749, 189750, 189751, 189752, 189753, 189754 were selected from the ORFs having the nucleotide sequence represented by SEQ ID NO: 174507.

The probes having the nucleotide sequence represented by SEQ ID NOS: 189755, 189756, 189757, 189758, 189759, 189760, 189761, 189762, 189763, 189764, 189765 were selected from the ORFs having the nucleotide sequence represented by SEQ ID NO: 174508.

The probes having the nucleotide sequence represented by SEQ ID NOS: 189766, 189767, 189768, 189769, 189770, 189771, 189772, 189773, 189774, 189775, 189776 were selected from the ORFs having the nucleotide sequence represented by SEQ ID NO: 174509.

The probes having the nucleotide sequence represented by SEQ ID NOS: 189777, 189778, 189779, 189780, 189781, 189782, 189783, 189784, 189785, 189786, 189787 were selected from the ORFs having the nucleotide sequence represented by SEQ ID NO: 174510.

The probes having the nucleotide sequence represented by SEQ ID NOS: 189788, 189789, 189790, 189791, 189792, 189793, 189794, 189795, 189796, 189797, 189798 were selected from the ORFs having the nucleotide sequence represented by SEQ ID NO: 174511.

The probes having the nucleotide sequence represented by SEQ ID NOS: 189799, 189800, 189801, 189802, 189803, 189804, 189805, 189806, 189807, 189808, 189809 were selected from the ORFs having the nucleotide sequence represented by SEQ ID NO: 174512.

The probes having the nucleotide sequence represented by SEQ ID NOS: 189810, 189811, 189812, 189813, 189814, 189815, 189816, 189817, 189818, 189819, 189820 were selected from the ORFs having the nucleotide sequence represented by SEQ ID NO: 174513.

The probes having the nucleotide sequence represented by SEQ ID NOS: 189821, 189822, 189823, 189824, 189825, 189826, 189827, 189828, 189829, 189830, 189831 were selected from the ORFs having the nucleotide sequence represented by SEQ ID NO: 174514.

The probes having the nucleotide sequence represented by SEQ ID NOS: 189832, 189833, 189834, 189835, 189836, 189837, 189838, 189839, 189840, 189841, 189842 were selected from the ORFs having the nucleotide sequence represented by SEQ ID NO: 174515.

The probes having the nucleotide sequence represented by SEQ ID NOS: 189843, 189844, 189845, 189846, 189847, 189848, 189849, 189850, 189851, 189852, 189853 were selected from the ORFs having the nucleotide sequence represented by SEQ ID NO: 174516.

The probes having the nucleotide sequence represented by SEQ ID NOS: 189854, 189855, 189856, 189857, 189858, 189859, 189860, 189861, 189862, 189863, 189864 were selected from the ORFs having the nucleotide sequence represented by SEQ ID NO: 174517.

The probes having the nucleotide sequence represented by SEQ ID NOS: 189865, 189866, 189867, 189868, 189869, 189870, 189871, 189872, 189873, 189874, 189875 were selected from the ORFs having the nucleotide sequence represented by SEQ ID NO: 174518.

The probes having the nucleotide sequence represented by SEQ ID NOS: 189876, 189877, 189878, 189879, 189880, 189881, 189882, 189883, 189884, 189885, 189886 were selected from the ORFs having the nucleotide sequence represented by SEQ ID NO: 174519.

The probes having the nucleotide sequence represented by SEQ ID NOS: 189887, 189888, 189889, 189890, 189891, 189892, 189893, 189894, 189895, 189896, 189897 were selected from the ORFs having the nucleotide sequence represented by SEQ ID NO: 174520.

The probes having the nucleotide sequence represented by SEQ ID NOS: 189898, 189899, 189900, 189901, 189902, 189903, 189904, 189905, 189906, 189907, 189908 were selected from the ORFs having the nucleotide sequence represented by SEQ ID NO: 174521.

The probes having the nucleotide sequence represented by SEQ ID NOS: 189909, 189910, 189911, 189912, 189913, 189914, 189915, 189916, 189917, 189918, 189919 were selected from the ORFs having the nucleotide sequence represented by SEQ ID NO: 174522.

The probes having the nucleotide sequence represented by SEQ ID NOS: 189920, 189921, 189922, 189923, 189924, 189925, 189926, 189927, 189928, 189929, 189930 were selected from the ORFs having the nucleotide sequence represented by SEQ ID NO: 174523.

The probes having the nucleotide sequence represented by SEQ ID NOS: 189931, 189932, 189933, 189934, 189935, 189936, 189937, 189938, 189939, 189940, 189941 were selected from the ORFs having the nucleotide sequence represented by SEQ ID NO: 174524.

The probes having the nucleotide sequence represented by SEQ ID NOS: 189942, 189943, 189944, 189945, 189946, 189947, 189948, 189949, 189950, 189951, 189952 were selected from the ORFs having the nucleotide sequence represented by SEQ ID NO: 174525.

The probes having the nucleotide sequence represented by SEQ ID NOS: 189953, 189954, 189955, 189956, 189957, 189958, 189959, 189960, 189961, 189962, 189963 were selected from the ORFs having the nucleotide sequence represented by SEQ ID NO: 174526.

The probes having the nucleotide sequence represented by SEQ ID NOS: 189964, 189965, 189966, 189967, 189968, 189969, 189970, 189971, 189972, 189973, 189974 were selected from the ORFs having the nucleotide sequence represented by SEQ ID NO: 174527.

The probes having the nucleotide sequence represented by SEQ ID NOS: 189975, 189976, 189977, 189978, 189979, 189980, 189981, 189982, 189983, 189984, 189985 were selected from the ORFs having the nucleotide sequence represented by SEQ ID NO: 174528.

The probes having the nucleotide sequence represented by SEQ ID NOS: 189986, 189987, 189988, 189989, 189990, 189991, 189992, 189993, 189994, 189995, 189996 were selected from the ORFs having the nucleotide sequence represented by SEQ ID NO: 174529.

The probes having the nucleotide sequence represented by SEQ ID NOS: 189997, 189998, 189999, 190000, 190001, 190002, 190003, 190004, 190005, 190006, 190007 were selected from the ORFs having the nucleotide sequence represented by SEQ ID NO: 174530.

The probes having the nucleotide sequence represented by SEQ ID NOS: 190008, 190009, 190010, 190011, 190012, 190013, 190014, 190015, 190016, 190017, 190018 were selected from the ORFs having the nucleotide sequence represented by SEQ ID NO: 174531.

The probes having the nucleotide sequence represented by SEQ ID NOS: 190019, 190020, 190021, 190022, 190023, 190024, 190025, 190026, 190027, 190028, 190029 were selected from the ORFs having the nucleotide sequence represented by SEQ ID NO: 174532.

The probes having the nucleotide sequence represented by SEQ ID NOS: 190030, 190031, 190032, 190033, 190034, 190035, 190036, 190037, 190038, 190039, 190040 were selected from the ORFs having the nucleotide sequence represented by SEQ ID NO: 174533.

The probes having the nucleotide sequence represented by SEQ ID NOS: 190041, 190042, 190043, 190044, 190045, 190046, 190047, 190048, 190049, 190050, 190051 were selected from the ORFs having the nucleotide sequence represented by SEQ ID NO: 174534.

The probes having the nucleotide sequence represented by SEQ ID NOS: 190052, 190053, 190054, 190055, 190056, 190057, 190058, 190059, 190060, 190061, 190062 were selected from the ORFs having the nucleotide sequence represented by SEQ ID NO: 174535.

The probes having the nucleotide sequence represented by SEQ ID NOS: 190063, 190064, 190065, 190066, 190067, 190068, 190069, 190070, 190071, 190072, 190073 were selected from the ORFs having the nucleotide sequence represented by SEQ ID NO: 174536.

The probes having the nucleotide sequence represented by SEQ ID NOS: 190074, 190075, 190076, 190077, 190078, 190079, 190080, 190081, 190082, 190083, 190084 were selected from the ORFs having the nucleotide sequence represented by SEQ ID NO: 174537.

The probes having the nucleotide sequence represented by SEQ ID NOS: 190085, 190086, 190087, 190088, 190089, 190090, 190091, 190092, 190093, 190094, 190095 were selected from the ORFs having the nucleotide sequence represented by SEQ ID NO: 174538.

The probes having the nucleotide sequence represented by SEQ ID NOS: 190096, 190097, 190098, 190099, 190100, 190101, 190102, 190103, 190104, 190105, 190106 were selected from the ORFs having the nucleotide sequence represented by SEQ ID NO: 174539.

The probes having the nucleotide sequence represented by SEQ ID NOS: 190107, 190108, 190109, 190110, 190111, 190112, 190113, 190114, 190115, 190116, 190117 were selected from the ORFs having the nucleotide sequence represented by SEQ ID NO: 174540.

The probes having the nucleotide sequence represented by SEQ ID NOS: 190118, 190119, 190120, 190121, 190122, 190123, 190124, 190125, 190126, 190127, 190128 were selected from the ORFs having the nucleotide sequence represented by SEQ ID NO: 174541.

The probes having the nucleotide sequence represented by SEQ ID NOS: 190129, 190130, 190131, 190132, 190133, 190134, 190135, 190136, 190137, 190138, 190139 were selected from the ORFs having the nucleotide sequence represented by SEQ ID NO: 174542.

The probes having the nucleotide sequence represented by SEQ ID NOS: 190140, 190141, 190142, 190143, 190144, 190145, 190146, 190147, 190148, 190149, 190150 were selected from the ORFs having the nucleotide sequence represented by SEQ ID NO: 174543.

The probes having the nucleotide sequence represented by SEQ ID NOS: 190151, 190152, 190153, 190154, 190155, 190156, 190157, 190158, 190159, 190160, 190161 were selected from the ORFs having the nucleotide sequence represented by SEQ ID NO: 174544.

The probes having the nucleotide sequence represented by SEQ ID NOS: 190162, 190163, 190164, 190165, 190166, 190167, 190168, 190169, 190170, 190171, 190172 were selected from the ORFs having the nucleotide sequence represented by SEQ ID NO: 174545.

The probes having the nucleotide sequence represented by SEQ ID NOS: 190173, 190174, 190175, 190176, 190177, 190178, 190179, 190180, 190181, 190182, 190183 were selected from the ORFs having the nucleotide sequence represented by SEQ ID NO: 174546.

The probes having the nucleotide sequence represented by SEQ ID NOS: 190184, 190185, 190186, 190187, 190188, 190189, 190190, 190191, 190192, 190193, 190194 were selected from the ORFs having the nucleotide sequence represented by SEQ ID NO: 174547.

The probes having the nucleotide sequence represented by SEQ ID NOS: 190195, 190196, 190197, 190198, 190199, 190200, 190201, 190202, 190203, 190204, 190205 were selected from the ORFs having the nucleotide sequence represented by SEQ ID NO: 174548.

The probes having the nucleotide sequence represented by SEQ ID NOS: 190206, 190207, 190208, 190209, 190210, 190211, 190212, 190213, 190214, 190215, 190216 were selected from the ORFs having the nucleotide sequence represented by SEQ ID NO: 174549.

The probes having the nucleotide sequence represented by SEQ ID NOS: 190217, 190218, 190219, 190220, 190221, 190222, 190223, 190224, 190225, 190226, 190227 were selected from the ORFs having the nucleotide sequence represented by SEQ ID NO: 174550.

The probes having the nucleotide sequence represented by SEQ ID NOS: 190228, 190229, 190230, 190231, 190232, 190233, 190234, 190235, 190236, 190237, 190238 were selected from the ORFs having the nucleotide sequence represented by SEQ ID NO: 174551.

The probes having the nucleotide sequence represented by SEQ ID NOS: 190239, 190240, 190241, 190242, 190243, 190244, 190245, 190246, 190247, 190248, 190249 were selected from the ORFs having the nucleotide sequence represented by SEQ ID NO: 174552.

The probes having the nucleotide sequence represented by SEQ ID NOS: 190250, 190251, 190252, 190253, 190254, 190255, 190256, 190257, 190258, 190259, 190260 were selected from the ORFs having the nucleotide sequence represented by SEQ ID NO: 174553.

The probes having the nucleotide sequence represented by SEQ ID NOS: 190261, 190262, 190263, 190264, 190265, 190266, 190267, 190268, 190269, 190270, 190271 were selected from the ORFs having the nucleotide sequence represented by SEQ ID NO: 174554.

The probes having the nucleotide sequence represented by SEQ ID NOS: 190272, 190273, 190274, 190275, 190276, 190277, 190278, 190279, 190280, 190281, 190282 were selected from the ORFs having the nucleotide sequence represented by SEQ ID NO: 174555.

The probes having the nucleotide sequence represented by SEQ ID NOS: 190283, 190284, 190285, 190286, 190287, 190288, 190289, 190290, 190291, 190292, 190293 were selected from the ORFs having the nucleotide sequence represented by SEQ ID NO: 174556.

The probes having the nucleotide sequence represented by SEQ ID NOS: 190294, 190295, 190296, 190297, 190298, 190299, 190300, 190301, 190302, 190303, 190304 were selected from the ORFs having the nucleotide sequence represented by SEQ ID NO: 174557.

The probes having the nucleotide sequence represented by SEQ ID NOS: 190305, 190306, 190307, 190308, 190309, 190310, 190311, 190312, 190313, 190314, 190315 were selected from the ORFs having the nucleotide sequence represented by SEQ ID NO: 174558.

The probes having the nucleotide sequence represented by SEQ ID NOS: 190316, 190317, 190318, 190319, 190320, 190321, 190322, 190323, 190324, 190325, 190326 were selected from the ORFs having the nucleotide sequence represented by SEQ ID NO: 174559.

The probes having the nucleotide sequence represented by SEQ ID NOS: 190327, 190328, 190329, 190330, 190331, 190332, 190333, 190334, 190335, 190336, 190337 were selected from the ORFs having the nucleotide sequence represented by SEQ ID NO: 174560.

The probes having the nucleotide sequence represented by SEQ ID NOS: 190338, 190339, 190340, 190341, 190342, 190343, 190344, 190345, 190346, 190347, 190348 were selected from the ORFs having the nucleotide sequence represented by SEQ ID NO: 174561.

The probes having the nucleotide sequence represented by SEQ ID NOS: 190349, 190350, 190351, 190352, 190353, 190354, 190355, 190356, 190357, 190358, 190359 were selected from the ORFs having the nucleotide sequence represented by SEQ ID NO: 174562.

The probes having the nucleotide sequence represented by SEQ ID NOS: 190360, 190361, 190362, 190363, 190364, 190365, 190366, 190367, 190368, 190369, 190370 were selected from the ORFs having the nucleotide sequence represented by SEQ ID NO: 174563.

The probes having the nucleotide sequence represented by SEQ ID NOS: 190371, 190372, 190373, 190374, 190375, 190376, 190377, 190378, 190379, 190380, 190381 were selected from the ORFs having the nucleotide sequence represented by SEQ ID NO: 174564.

The probes having the nucleotide sequence represented by SEQ ID NOS: 190382, 190383, 190384, 190385, 190386, 190387, 190388, 190389, 190390, 190391, 190392 were selected from the ORFs having the nucleotide sequence represented by SEQ ID NO: 174565.

The probes having the nucleotide sequence represented by SEQ ID NOS: 190393, 190394, 190395, 190396, 190397, 190398, 190399, 190400, 190401, 190402, 190403 were selected from the ORFs having the nucleotide sequence represented by SEQ ID NO: 174566.

The probes having the nucleotide sequence represented by SEQ ID NOS: 190404, 190405, 190406, 190407, 190408, 190409, 190410, 190411, 190412, 190413, 190414 were selected from the ORFs having the nucleotide sequence represented by SEQ ID NO: 174567.

The probes having the nucleotide sequence represented by SEQ ID NOS: 190415, 190416, 190417, 190418, 190419, 190420, 190421, 190422, 190423, 190424, 190425 were selected from the ORFs having the nucleotide sequence represented by SEQ ID NO: 174568.

The probes having the nucleotide sequence represented by SEQ ID NOS: 190426, 190427, 190428, 190429, 190430, 190431, 190432, 190433, 190434, 190435, 190436 were selected from the ORFs having the nucleotide sequence represented by SEQ ID NO: 174569.

The probes having the nucleotide sequence represented by SEQ ID NOS: 190437, 190438, 190439, 190440, 190441, 190442, 190443, 190444, 190445, 190446, 190447 were selected from the ORFs having the nucleotide sequence represented by SEQ ID NO: 174570.

The probes having the nucleotide sequence represented by SEQ ID NOS: 190448, 190449, 190450, 190451, 190452, 190453, 190454, 190455, 190456, 190457, 190458 were selected from the ORFs having the nucleotide sequence represented by SEQ ID NO: 174571.

The probes having the nucleotide sequence represented by SEQ ID NOS: 190459, 190460, 190461, 190462, 190463, 190464, 190465, 190466, 190467, 190468, 190469 were selected from the ORFs having the nucleotide sequence represented by SEQ ID NO: 174572.

The probes having the nucleotide sequence represented by SEQ ID NOS: 190470, 190471, 190472, 190473, 190474, 190475, 190476, 190477, 190478, 190479, 190480 were selected from the ORFs having the nucleotide sequence represented by SEQ ID NO: 174573.

The probes having the nucleotide sequence represented by SEQ ID NOS: 190481, 190482, 190483, 190484, 190485, 190486, 190487, 190488, 190489, 190490, 190491 were selected from the ORFs having the nucleotide sequence represented by SEQ ID NO: 174574.

The probes having the nucleotide sequence represented by SEQ ID NOS: 190492, 190493, 190494, 190495, 190496, 190497, 190498, 190499, 190500, 190501, 190502 were selected from the ORFs having the nucleotide sequence represented by SEQ ID NO: 174575.

The probes having the nucleotide sequence represented by SEQ ID NOS: 190503, 190504, 190505, 190506, 190507, 190508, 190509, 190510, 190511, 190512, 190513 were selected from the ORFs having the nucleotide sequence represented by SEQ ID NO: 174576.

The probes having the nucleotide sequence represented by SEQ ID NOS: 190514, 190515, 190516, 190517, 190518, 190519, 190520, 190521, 190522, 190523, 190524 were selected from the ORFs having the nucleotide sequence represented by SEQ ID NO: 174577.

The probes having the nucleotide sequence represented by SEQ ID NOS: 190525, 190526, 190527, 190528, 190529, 190530, 190531, 190532, 190533, 190534, 190535 were selected from the ORFs having the nucleotide sequence represented by SEQ ID NO: 174578.

The probes having the nucleotide sequence represented by SEQ ID NOS: 190536, 190537, 190538, 190539, 190540, 190541, 190542, 190543, 190544, 190545, 190546 were selected from the ORFs having the nucleotide sequence represented by SEQ ID NO: 174579.

The probes having the nucleotide sequence represented by SEQ ID NOS: 190547, 190548, 190549, 190550, 190551, 190552, 190553, 190554, 190555, 190556, 190557 were selected from the ORFs having the nucleotide sequence represented by SEQ ID NO: 174580.

The probes having the nucleotide sequence represented by SEQ ID NOS: 190558, 190559, 190560, 190561, 190562, 190563, 190564, 190565, 190566, 190567, 190568 were selected from the ORFs having the nucleotide sequence represented by SEQ ID NO: 174581.

The probes having the nucleotide sequence represented by SEQ ID NOS: 190569, 190570, 190571, 190572, 190573, 190574, 190575, 190576, 190577, 190578, 190579 were selected from the ORFs having the nucleotide sequence represented by SEQ ID NO: 174582.

The probes having the nucleotide sequence represented by SEQ ID NOS: 190580, 190581, 190582, 190583, 190584, 190585, 190586, 190587, 190588, 190589, 190590 were selected from the ORFs having the nucleotide sequence represented by SEQ ID NO: 174583.

The probes having the nucleotide sequence represented by SEQ ID NOS: 190591, 190592, 190593, 190594, 190595, 190596, 190597, 190598, 190599, 190600, 190601 were selected from the ORFs having the nucleotide sequence represented by SEQ ID NO: 174584.

The probes having the nucleotide sequence represented by SEQ ID NOS: 190602, 190603, 190604, 190605, 190606, 190607, 190608, 190609, 190610, 190611, 190612 were selected from the ORFs having the nucleotide sequence represented by SEQ ID NO: 174585.

The probes having the nucleotide sequence represented by SEQ ID NOS: 190613, 190614, 190615, 190616, 190617, 190618, 190619, 190620, 190621, 190622, 190623 were selected from the ORFs having the nucleotide sequence represented by SEQ ID NO: 174586.

The probes having the nucleotide sequence represented by SEQ ID NOS: 190624, 190625, 190626, 190627, 190628, 190629, 190630, 190631, 190632, 190633, 190634 were selected from the ORFs having the nucleotide sequence represented by SEQ ID NO: 174587.

The probes having the nucleotide sequence represented by SEQ ID NOS: 190635, 190636, 190637, 190638, 190639, 190640, 190641, 190642, 190643, 190644, 190645 were selected from the ORFs having the nucleotide sequence represented by SEQ ID NO: 174588.

The probes having the nucleotide sequence represented by SEQ ID NOS: 190646, 190647, 190648, 190649, 190650, 190651, 190652, 190653, 190654, 190655, 190656 were selected from the ORFs having the nucleotide sequence represented by SEQ ID NO: 174589.

The probes having the nucleotide sequence represented by SEQ ID NOS: 190657, 190658, 190659, 190660, 190661, 190662, 190663, 190664, 190665, 190666, 190667 were selected from the ORFs having the nucleotide sequence represented by SEQ ID NO: 174590.

The probes having the nucleotide sequence represented by SEQ ID NOS: 190668, 190669, 190670, 190671, 190672, 190673, 190674, 190675, 190676, 190677, 190678 were selected from the ORFs having the nucleotide sequence represented by SEQ ID NO: 174591.

The probes having the nucleotide sequence represented by SEQ ID NOS: 190679, 190680, 190681, 190682, 190683, 190684, 190685, 190686, 190687, 190688, 190689 were selected from the ORFs having the nucleotide sequence represented by SEQ ID NO: 174592.

The probes having the nucleotide sequence represented by SEQ ID NOS: 190690, 190691, 190692, 190693, 190694, 190695, 190696, 190697, 190698, 190699, 190700 were selected from the ORFs having the nucleotide sequence represented by SEQ ID NO: 174593.

The probes having the nucleotide sequence represented by SEQ ID NOS: 190701, 190702, 190703, 190704, 190705, 190706, 190707, 190708, 190709, 190710, 190711 were selected from the ORFs having the nucleotide sequence represented by SEQ ID NO: 174594.

The probes having the nucleotide sequence represented by SEQ ID NOS: 190712, 190713, 190714, 190715, 190716, 190717, 190718, 190719, 190720, 190721, 190722 were selected from the ORFs having the nucleotide sequence represented by SEQ ID NO: 174595.

The probes having the nucleotide sequence represented by SEQ ID NOS: 190723, 190724, 190725, 190726, 190727, 190728, 190729, 190730, 190731, 190732, 190733 were selected from the ORFs having the nucleotide sequence represented by SEQ ID NO: 174596.

The probes having the nucleotide sequence represented by SEQ ID NOS: 190734, 190735, 190736, 190737, 190738, 190739, 190740, 190741, 190742, 190743, 190744 were selected from the ORFs having the nucleotide sequence represented by SEQ ID NO: 174597.

The probes having the nucleotide sequence represented by SEQ ID NOS: 190745, 190746, 190747, 190748, 190749, 190750, 190751, 190752, 190753, 190754, 190755 were selected from the ORFs having the nucleotide sequence represented by SEQ ID NO: 174598.

The probes having the nucleotide sequence represented by SEQ ID NOS: 190756, 190757, 190758, 190759, 190760, 190761, 190762, 190763, 190764, 190765, 190766 were selected from the ORFs having the nucleotide sequence represented by SEQ ID NO: 174599.

The probes having the nucleotide sequence represented by SEQ ID NOS: 190767, 190768, 190769, 190770, 190771, 190772, 190773, 190774, 190775, 190776, 190777 were selected from the ORFs having the nucleotide sequence represented by SEQ ID NO: 174600.

The probes having the nucleotide sequence represented by SEQ ID NOS: 190778, 190779, 190780, 190781, 190782, 190783, 190784, 190785, 190786, 190787, 190788 were selected from the ORFs having the nucleotide sequence represented by SEQ ID NO: 174601.

The probes having the nucleotide sequence represented by SEQ ID NOS: 190789, 190790, 190791, 190792, 190793, 190794, 190795, 190796, 190797, 190798, 190799 were selected from the ORFs having the nucleotide sequence represented by SEQ ID NO: 174602.

The probes having the nucleotide sequence represented by SEQ ID NOS: 190800, 190801, 190802, 190803, 190804, 190805, 190806, 190807, 190808, 190809, 190810 were selected from the ORFs having the nucleotide sequence represented by SEQ ID NO: 174603.

The probes from 403 S. cerevisiae ORFs described as above (2) were as follows:

The probes having the nucleotide sequence represented by SEQ ID NOS: 191214, 191215, 191216, 191217, 191218, 191219, 191220, 191221, 191222, 191223, 191224 were selected from the ORFs having the nucleotide sequence represented by SEQ ID NO: 190811.

The probes having the nucleotide sequence represented by SEQ ID NOS: 191225, 191226, 191227, 191228, 191229, 191230, 191231, 191232, 191233, 191234, 191235 were selected from the ORFs having the nucleotide sequence represented by SEQ ID NO: 190812.

The probes having the nucleotide sequence represented by SEQ ID NOS: 191236, 191237, 191238, 191239, 191240, 191241, 191242, 191243, 191244, 191245, 191246 were selected from the ORFs having the nucleotide sequence represented by SEQ ID NO: 190813.

The probes having the nucleotide sequence represented by SEQ ID NOS: 191247, 191248, 191249, 191250, 191251, 191252, 191253, 191254, 191255, 191256, 191257 were selected from the ORFs having the nucleotide sequence represented by SEQ ID NO: 190814.

The probes having the nucleotide sequence represented by SEQ ID NOS: 191258, 191259, 191260, 191261, 191262, 191263, 191264, 191265, 191266, 191267, 191268 were selected from the ORFs having the nucleotide sequence represented by SEQ ID NO: 190815.

The probes having the nucleotide sequence represented by SEQ ID NOS: 191269, 191270, 191271, 191272, 191273, 191274, 191275, 191276, 191277, 191278, 191279 were selected from the ORFs having the nucleotide sequence represented by SEQ ID NO: 190816.

The probes having the nucleotide sequence represented by SEQ ID NOS: 191280, 191281, 191282, 191283, 191284, 191285, 191286, 191287, 191288, 191289, 191290 were selected from the ORFs having the nucleotide sequence represented by SEQ ID NO: 190817.

The probes having the nucleotide sequence represented by SEQ ID NOS: 191291, 191292, 191293, 191294, 191295, 191296, 191297, 191298, 191299, 191300, 191301 were selected from the ORFs having the nucleotide sequence represented by SEQ ID NO: 190818.

The probes having the nucleotide sequence represented by SEQ ID NOS: 191302, 191303, 191304, 191305, 191306, 191307, 191308, 191309, 191310, 191311, 191312 were selected from the ORFs having the nucleotide sequence represented by SEQ ID NO: 190819.

The probes having the nucleotide sequence represented by SEQ ID NOS: 191313, 191314, 191315, 191316, 191317, 191318, 191319, 191320, 191321, 191322, 191323 were selected from the ORFs having the nucleotide sequence represented by SEQ ID NO: 190820.

The probes having the nucleotide sequence represented by SEQ ID NOS: 191324, 191325, 191326, 191327, 191328, 191329, 191330, 191331, 191332, 191333, 191334 were selected from the ORFs having the nucleotide sequence represented by SEQ ID NO: 190821.

The probes having the nucleotide sequence represented by SEQ ID NOS: 191335, 191336, 191337, 191338, 191339, 191340, 191341, 191342, 191343, 191344, 191345 were selected from the ORFs having the nucleotide sequence represented by SEQ ID NO: 190822.

The probes having the nucleotide sequence represented by SEQ ID NOS: 191346, 191347, 191348, 191349, 191350, 191351, 191352, 191353, 191354, 191355, 191356 were selected from the ORFs having the nucleotide sequence represented by SEQ ID NO: 190823.

The probes having the nucleotide sequence represented by SEQ ID NOS: 191357, 191358, 191359, 191360, 191361, 191362, 191363, 191364, 191365, 191366, 191367 were selected from the ORFs having the nucleotide sequence represented by SEQ ID NO: 190824.

The probes having the nucleotide sequence represented by SEQ ID NOS: 191368, 191369, 191370, 191371, 191372, 191373, 191374, 191375, 191376, 191377, 191378 were selected from the ORFs having the nucleotide sequence represented by SEQ ID NO: 190825.

The probes having the nucleotide sequence represented by SEQ ID NOS: 191379, 191380, 191381, 191382, 191383, 191384, 191385, 191386, 191387, 191388, 191389 were selected from the ORFs having the nucleotide sequence represented by SEQ ID NO: 190826.

The probes having the nucleotide sequence represented by SEQ ID NOS: 191390, 191391, 191392, 191393, 191394, 191395, 191396, 191397, 191398, 191399, 191400 were selected from the ORFs having the nucleotide sequence represented by SEQ ID NO: 190827.

The probes having the nucleotide sequence represented by SEQ ID NOS: 191401, 191402, 191403, 191404, 191405, 191406, 191407, 191408, 191409, 191410, 191411 were selected from the ORFs having the nucleotide sequence represented by SEQ ID NO: 190828.

The probes having the nucleotide sequence represented by SEQ ID NOS: 191412, 191413, 191414, 191415, 191416, 191417, 191418, 191419, 191420, 191421, 191422 were selected from the ORFs having the nucleotide sequence represented by SEQ ID NO: 190829.

The probes having the nucleotide sequence represented by SEQ ID NOS: 191423, 191424, 191425, 191426, 191427, 191428, 191429, 191430, 191431, 191432, 191433 were selected from the ORFs having the nucleotide sequence represented by SEQ ID NO: 190830.

The probes having the nucleotide sequence represented by SEQ ID NOS: 191434, 191435, 191436, 191437, 191438, 191439, 191440, 191441, 191442, 191443, 191444 were selected from the ORFs having the nucleotide sequence represented by SEQ ID NO: 190831.

The probes having the nucleotide sequence represented by SEQ ID NOS: 191445, 191446, 191447, 191448, 191449, 191450, 191451, 191452, 191453, 191454, 191455 were selected from the ORFs having the nucleotide sequence represented by SEQ ID NO: 190832.

The probes having the nucleotide sequence represented by SEQ ID NOS: 191456, 191457, 191458, 191459, 191460, 191461, 191462, 191463, 191464, 191465, 191466 were selected from the ORFs having the nucleotide sequence represented by SEQ ID NO: 190833.

The probes having the nucleotide sequence represented by SEQ ID NOS: 191467, 191468, 191469, 191470, 191471, 191472, 191473, 191474, 191475, 191476, 191477 were selected from the ORFs having the nucleotide sequence represented by SEQ ID NO: 190834.

The probes having the nucleotide sequence represented by SEQ ID NOS: 191478, 191479, 191480, 191481, 191482, 191483, 191484, 191485 were selected from the ORFs having the nucleotide sequence represented by SEQ ID NO: 190835.

The probes having the nucleotide sequence represented by SEQ ID NOS: 191486, 191487, 191488, 191489, 191490, 191491, 191492, 191493, 191494, 191495, 191496 were selected from the ORFs having the nucleotide sequence represented by SEQ ID NO: 190836.

The probes having the nucleotide sequence represented by SEQ ID NOS: 191497, 191498, 191499, 191500, 191501, 191502, 191503, 191504, 191505, 191506, 191507 were selected from the ORFs having the nucleotide sequence represented by SEQ ID NO: 190837.

The probes having the nucleotide sequence represented by SEQ ID NOS: 191508, 191509, 191510, 191511, 191512, 191513, 191514, 191515, 191516, 191517, 191518 were selected from the ORFs having the nucleotide sequence represented by SEQ ID NO: 190838.

The probes having the nucleotide sequence represented by SEQ ID NOS: 191519, 191520, 191521, 191522, 191523, 191524, 191525, 191526, 191527, 191528, 191529 were selected from the ORFs having the nucleotide sequence represented by SEQ ID NO: 190839.

The probes having the nucleotide sequence represented by SEQ ID NOS: 191530, 191531, 191532, 191533, 191534, 191535, 191536, 191537, 191538, 191539, 191540 were selected from the ORFs having the nucleotide sequence represented by SEQ ID NO: 190840.

The probes having the nucleotide sequence represented by SEQ ID NOS: 191541, 191542, 191543, 191544, 191545, 191546, 191547, 191548 were selected from the ORFs having the nucleotide sequence represented by SEQ ID NO: 190841.

The probes having the nucleotide sequence represented by SEQ ID NOS: 191549, 191550, 191551, 191552, 191553, 191554, 191555, 191556, 191557, 191558, 191559 were selected from the ORFs having the nucleotide sequence represented by SEQ ID NO: 190842.

The probes having the nucleotide sequence represented by SEQ ID NOS: 191560, 191561, 191562, 191563, 191564, 191565, 191566, 191567 were selected from the ORFs having the nucleotide sequence represented by SEQ ID NO: 190843.

The probes having the nucleotide sequence represented by SEQ ID NOS: 191568, 191569, 191570, 191571, 191572, 191573, 191574, 191575, 191576, 191577, 191578 were selected from the ORFs having the nucleotide sequence represented by SEQ ID NO: 190844.

The probes having the nucleotide sequence represented by SEQ ID NOS: 191579, 191580, 191581, 191582, 191583, 191584, 191585, 191586, 191587, 191588, 191589 were selected from the ORFs having the nucleotide sequence represented by SEQ ID NO: 190845.

The probes having the nucleotide sequence represented by SEQ ID NOS: 191590, 191591, 191592, 191593, 191594, 191595, 191596, 191597, 191598, 191599, 191600 were selected from the ORFs having the nucleotide sequence represented by SEQ ID NO: 190846.

The probes having the nucleotide sequence represented by SEQ ID NOS: 191601, 191602, 191603, 191604, 191605, 191606, 191607, 191608, 191609, 191610, 191611 were selected from the ORFs having the nucleotide sequence represented by SEQ ID NO: 190847.

The probes having the nucleotide sequence represented by SEQ ID NOS: 191612, 191613, 191614, 191615, 191616, 191617, 191618, 191619, 191620, 191621, 191622 were selected from the ORFs having the nucleotide sequence represented by SEQ ID NO: 190848.

The probes having the nucleotide sequence represented by SEQ ID NOS: 191623, 191624, 191625, 191626, 191627, 191628, 191629, 191630, 191631, 191632, 191633 were selected from the ORFs having the nucleotide sequence represented by SEQ ID NO: 190849.

The probes having the nucleotide sequence represented by SEQ ID NOS: 191634, 191635, 191636, 191637, 191638, 191639, 191640, 191641, 191642, 191643, 191644 were selected from the ORFs having the nucleotide sequence represented by SEQ ID NO: 190850.

The probes having the nucleotide sequence represented by SEQ ID NOS: 191645, 191646, 191647, 191648, 191649, 191650, 191651, 191652, 191653, 191654, 191655 were selected from the ORFs having the nucleotide sequence represented by SEQ ID NO: 190851.

The probes having the nucleotide sequence represented by SEQ ID NOS: 191656, 191657, 191658, 191659, 191660, 191661, 191662, 191663, 191664, 191665, 191666 were selected from the ORFs having the nucleotide sequence represented by SEQ ID NO: 190852.

The probes having the nucleotide sequence represented by SEQ ID NOS: 191667, 191668, 191669, 191670, 191671, 191672, 191673, 191674, 191675, 191676, 191677 were selected from the ORFs having the nucleotide sequence represented by SEQ ID NO: 190853.

The probes having the nucleotide sequence represented by SEQ ID NOS: 191678, 191679, 191680, 191681, 191682, 191683, 191684, 191685, 191686, 191687, 191688 were selected from the ORFs having the nucleotide sequence represented by SEQ ID NO: 190854.

The probes having the nucleotide sequence represented by SEQ ID NOS: 191689, 191690, 191691, 191692, 191693, 191694, 191695, 191696, 191697, 191698, 191699 were selected from the ORFs having the nucleotide sequence represented by SEQ ID NO: 190855.

The probes having the nucleotide sequence represented by SEQ ID NOS: 191700, 191701, 191702, 191703, 191704, 191705, 191706, 191707, 191708, 191709, 191710 were selected from the ORFs having the nucleotide sequence represented by SEQ ID NO: 190856.

The probes having the nucleotide sequence represented by SEQ ID NOS: 191711, 191712, 191713, 191714, 191715, 191716, 191717, 191718, 191719, 191720, 191721 were selected from the ORFs having the nucleotide sequence represented by SEQ ID NO: 190857.

The probes having the nucleotide sequence represented by SEQ ID NOS: 191722, 191723, 191724, 191725, 191726, 191727, 191728, 191729, 191730, 191731, 191732 were selected from the ORFs having the nucleotide sequence represented by SEQ ID NO: 190858.

The probes having the nucleotide sequence represented by SEQ ID NOS: 191733, 191734, 191735, 191736, 191737, 191738, 191739, 191740, 191741, 191742, 191743 were selected from the ORFs having the nucleotide sequence represented by SEQ ID NO: 190859.

The probes having the nucleotide sequence represented by SEQ ID NOS: 191744, 191745, 191746, 191747, 191748, 191749, 191750, 191751, 191752, 191753, 191754 were selected from the ORFs having the nucleotide sequence represented by SEQ ID NO: 190860.

The probes having the nucleotide sequence represented by SEQ ID NOS: 191755, 191756, 191757, 191758, 191759, 191760, 191761, 191762, 191763, 191764, 191765 were selected from the ORFs having the nucleotide sequence represented by SEQ ID NO: 190861.

The probes having the nucleotide sequence represented by SEQ ID NOS: 191766, 191767, 191768, 191769, 191770, 191771, 191772, 191773, 191774, 191775, 191776 were selected from the ORFs having the nucleotide sequence represented by SEQ ID NO: 190862.

The probes having the nucleotide sequence represented by SEQ ID NOS: 191777, 191778, 191779, 191780, 191781, 191782, 191783, 191784, 191785, 191786, 191787 were selected from the ORFs having the nucleotide sequence represented by SEQ ID NO: 190863.

The probes having the nucleotide sequence represented by SEQ ID NOS: 191788, 191789, 191790, 191791, 191792, 191793, 191794, 191795, 191796, 191797, 191798 were selected from the ORFs having the nucleotide sequence represented by SEQ ID NO: 190864.

The probes having the nucleotide sequence represented by SEQ ID NOS: 191799, 191800, 191801, 191802, 191803, 191804, 191805, 191806, 191807, 191808, 191809 were selected from the ORFs having the nucleotide sequence represented by SEQ ID NO: 190865.

The probes having the nucleotide sequence represented by SEQ ID NOS: 191810, 191811, 191812, 191813, 191814, 191815, 191816, 191817, 191818, 191819, 191820 were selected from the ORFs having the nucleotide sequence represented by SEQ ID NO: 190866.

The probes having the nucleotide sequence represented by SEQ ID NOS: 191821, 191822, 191823, 191824, 191825, 191826, 191827, 191828, 191829, 191830, 191831 were selected from the ORFs having the nucleotide sequence represented by SEQ ID NO: 190867.

The probes having the nucleotide sequence represented by SEQ ID NOS: 191832, 191833, 191834, 191835, 191836, 191837, 191838, 191839, 191840, 191841, 191842 were selected from the ORFs having the nucleotide sequence represented by SEQ ID NO: 190868.

The probes having the nucleotide sequence represented by SEQ ID NOS: 191843, 191844, 191845, 191846, 191847, 191848, 191849, 191850, 191851, 191852, 191853 were selected from the ORFs having the nucleotide sequence represented by SEQ ID NO: 190869.

The probes having the nucleotide sequence represented by SEQ ID NOS: 191854, 191855, 191856, 191857, 191858, 191859, 191860, 191861, 191862, 191863, 191864 were selected from the ORFs having the nucleotide sequence represented by SEQ ID NO: 190870.

The probes having the nucleotide sequence represented by SEQ ID NOS: 191865, 191866, 191867, 191868, 191869, 191870, 191871, 191872, 191873, 191874, 191875 were selected from the ORFs having the nucleotide sequence represented by SEQ ID NO: 190871.

The probes having the nucleotide sequence represented by SEQ ID NOS: 191876, 191877, 191878, 191879, 191880, 191881, 191882, 191883, 191884, 191885, 191886 were selected from the ORFs having the nucleotide sequence represented by SEQ ID NO: 190872.

The probes having the nucleotide sequence represented by SEQ ID NOS: 191887, 191888, 191889, 191890, 191891, 191892, 191893, 191894, 191895, 191896, 191897 were selected from the ORFs having the nucleotide sequence represented by SEQ ID NO: 190873.

The probes having the nucleotide sequence represented by SEQ ID NOS: 191898, 191899, 191900, 191901, 191902, 191903, 191904, 191905, 191906, 191907, 191908 were selected from the ORFs having the nucleotide sequence represented by SEQ ID NO: 190874.

The probes having the nucleotide sequence represented by SEQ ID NOS: 191909, 191910, 191911, 191912, 191913, 191914, 191915, 191916, 191917, 191918, 191919 were selected from the ORFs having the nucleotide sequence represented by SEQ ID NO: 190875.

The probes having the nucleotide sequence represented by SEQ ID NOS: 191920, 191921, 191922, 191923, 191924, 191925, 191926, 191927, 191928, 191929, 191930 were selected from the ORFs having the nucleotide sequence represented by SEQ ID NO: 190876.

The probes having the nucleotide sequence represented by SEQ ID NOS: 191931, 191932, 191933, 191934, 191935, 191936, 191937, 191938, 191939, 191940, 191941 were selected from the ORFs having the nucleotide sequence represented by SEQ ID NO: 190877.

The probes having the nucleotide sequence represented by SEQ ID NOS: 191942, 191943, 191944, 191945, 191946, 191947, 191948, 191949, 191950, 191951, 191952 were selected from the ORFs having the nucleotide sequence represented by SEQ ID NO: 190878.

The probes having the nucleotide sequence represented by SEQ ID NOS: 191953, 191954, 191955, 191956, 191957, 191958, 191959, 191960, 191961, 191962, 191963 were selected from the ORFs having the nucleotide sequence represented by SEQ ID NO: 190879.

The probes having the nucleotide sequence represented by SEQ ID NOS: 191964, 191965, 191966, 191967, 191968, 191969, 191970, 191971 were selected from the ORFs having the nucleotide sequence represented by SEQ ID NO: 190880.

The probes having the nucleotide sequence represented by SEQ ID NOS: 191972, 191973, 191974, 191975, 191976, 191977, 191978, 191979, 191980, 191981, 191982 were selected from the ORFs having the nucleotide sequence represented by SEQ ID NO: 190881.

The probes having the nucleotide sequence represented by SEQ ID NOS: 191983, 191984, 191985, 191986, 191987, 191988, 191989, 191990, 191991, 191992, 191993 were selected from the ORFs having the nucleotide sequence represented by SEQ ID NO: 190882.

The probes having the nucleotide sequence represented by SEQ ID NOS: 191994, 191995, 191996, 191997, 191998, 191999, 192000, 192001 were selected from the ORFs having the nucleotide sequence represented by SEQ ID NO: 190883.

The probes having the nucleotide sequence represented by SEQ ID NOS: 192002, 192003, 192004, 192005, 192006, 192007, 192008, 192009, 192010, 192011, 192012 were selected from the ORFs having the nucleotide sequence represented by SEQ ID NO: 190884.

The probes having the nucleotide sequence represented by SEQ ID NOS: 192013, 192014, 192015, 192016, 192017, 192018, 192019, 192020 were selected from the ORFs having the nucleotide sequence represented by SEQ ID NO:. 190885.

The probes having the nucleotide sequence represented by SEQ ID NOS: 192021, 192022, 192023, 192024, 192025, 192026, 192027, 192028 were selected from the ORFs having the nucleotide sequence represented by SEQ ID NO: 190886.

The probes having the nucleotide sequence represented by SEQ ID NOS: 192029, 192030, 192031, 192032, 192033, 192034, 192035, 192036, 192037, 192038, 192039 were selected from the ORFs having the nucleotide sequence represented by SEQ ID NO: 190887.

The probes having the nucleotide sequence represented by SEQ ID NOS: 192040, 192041, 192042, 192043, 192044, 192045, 192046, 192047, 192048, 192049, 192050 were selected from the ORFs having the nucleotide sequence represented by SEQ ID NO: 190888.

The probes having the nucleotide sequence represented by SEQ ID NOS: 192051, 192052, 192053, 192054, 192055, 192056, 192057, 192058, 192059, 192060, 192061 were selected from the ORFs having the nucleotide sequence represented by SEQ ID NO: 190889.

The probes having the nucleotide sequence represented by SEQ ID NOS: 192062, 192063, 192064, 192065, 192066, 192067, 192068, 192069, 192070, 192071, 192072 were selected from the ORFs having the nucleotide sequence represented by SEQ ID NO: 190890.

The probes having the nucleotide sequence represented by SEQ ID NOS: 192073, 192074, 192075, 192076, 192077, 192078, 192079, 192080, 192081, 192082, 192083 were selected from the ORFs having the nucleotide sequence represented by SEQ ID NO: 190891.

The probes having the nucleotide sequence represented by SEQ ID NOS: 192084, 192085, 192086, 192087, 192088, 192089, 192090, 192091, 192092, 192093, 192094 were selected from the ORFs having the nucleotide sequence represented by SEQ ID NO: 190892.

The probes having the nucleotide sequence represented by SEQ ID NOS: 192095, 192096, 192097, 192098, 192099, 192100, 192101, 192102, 192103, 192104, 192105 were selected from the ORFs having the nucleotide sequence represented by SEQ ID NO: 190893.

The probes having the nucleotide sequence represented by SEQ ID NOS: 192106, 192107, 192108, 192109, 192110, 192111, 192112, 192113, 192114, 192115, 192116 were selected from the ORFs having the nucleotide sequence represented by SEQ ID NO: 190894.

The probes having the nucleotide sequence represented by SEQ ID NOS: 192117, 192118, 192119, 192120, 192121, 192122, 192123, 192124, 192125, 192126, 192127 were selected from the ORFs having the nucleotide sequence represented by SEQ ID NO: 190895.

The probes having the nucleotide sequence represented by SEQ ID NOS: 192128, 192129, 192130, 192131, 192132, 192133, 192134, 192135, 192136, 192137, 192138 were selected from the ORFs having the nucleotide sequence represented by SEQ ID NO: 190896.

The probes having the nucleotide sequence represented by SEQ ID NOS: 192139, 192140, 192141, 192142, 192143, 192144, 192145, 192146, 192147, 192148, 192149 were selected from the ORFs having the nucleotide sequence represented by SEQ ID NO: 190897.

The probes having the nucleotide sequence represented by SEQ ID NOS: 192150, 192151, 192152, 192153, 192154, 192155, 192156, 192157, 192158, 192159, 192160 were selected from the ORFs having the nucleotide sequence represented by SEQ ID NO: 190898.

The probes having the nucleotide sequence represented by SEQ ID NOS: 192161, 192162, 192163, 192164, 192165, 192166, 192167, 192168 were selected from the ORFs having the nucleotide sequence represented by SEQ ID NO: 190899.

The probes having the nucleotide sequence represented by SEQ ID NOS: 192169, 192170, 192171, 192172, 192173, 192174, 192175, 192176, 192177, 192178, 192179 were selected from the ORFs having the nucleotide sequence represented by SEQ ID NO: 190900.

The probes having the nucleotide sequence represented by SEQ ID NOS: 192180, 192181, 192182, 192183, 192184, 192185, 192186, 192187, 192188, 192189, 192190 were selected from the ORFs having the nucleotide sequence represented by SEQ ID NO: 190901.

The probes having the nucleotide sequence represented by SEQ ID NOS: 192191, 192192, 192193, 192194, 192195, 192196, 192197, 192198, 192199, 192200, 192201 were selected from the ORFs having the nucleotide sequence represented by SEQ ID NO: 190902.

The probes having the nucleotide sequence represented by SEQ ID NOS: 192202, 192203, 192204, 192205, 192206, 192207, 192208, 192209, 192210, 192211, 192212 were selected from the ORFs having the nucleotide sequence represented by SEQ ID NO: 190903.

The probes having the nucleotide sequence represented by SEQ ID NOS: 192213, 192214, 192215, 192216, 192217, 192218, 192219, 192220, 192221, 192222, 192223 were selected from the ORFs having the nucleotide sequence represented by SEQ ID NO: 190904.

The probes having the nucleotide sequence represented by SEQ ID NOS: 192224, 192225, 192226, 192227, 192228, 192229, 192230, 192231, 192232, 192233, 192234 were selected from the ORFs having the nucleotide sequence represented by SEQ ID NO: 190905.

The probes having the nucleotide sequence represented by SEQ ID NOS: 192235, 192236, 192237, 192238, 192239, 192240, 192241, 192242, 192243, 192244, 192245 were selected from the ORFs having the nucleotide sequence represented by SEQ ID NO: 190906.

The probes having the nucleotide sequence represented by SEQ ID NOS: 192246, 192247, 192248, 192249, 192250, 192251, 192252, 192253 were selected from the ORFs having the nucleotide sequence represented by SEQ ID NO: 190907.

The probes having the nucleotide sequence represented by SEQ ID NOS: 192254, 192255, 192256, 192257, 192258, 192259, 192260, 192261, 192262, 192263, 192264 were selected from the ORFs having the nucleotide sequence represented by SEQ ID NO: 190908.

The probes having the nucleotide sequence represented by SEQ ID NOS: 192265, 192266, 192267, 192268, 192269, 192270, 192271, 192272, 192273, 192274, 192275 were selected from the ORFs having the nucleotide sequence represented by SEQ ID NO: 190909.

The probes having the nucleotide sequence represented by SEQ ID NOS: 192276, 192277, 192278, 192279, 192280, 192281, 192282, 192283, 192284, 192285, 192286 were selected from the ORFs having the nucleotide sequence represented by SEQ ID NO: 190910.

The probes having the nucleotide sequence represented by SEQ ID NOS: 192287, 192288, 192289, 192290, 192291, 192292, 192293, 192294, 192295, 192296, 192297 were selected from the ORFs having the nucleotide sequence represented by SEQ ID NO: 190911.

The probes having the nucleotide sequence represented by SEQ ID NOS: 192298, 192299, 192300, 192301, 192302, 192303, 192304, 192305, 192306, 192307, 192308 were selected from the ORFs having the nucleotide sequence represented by SEQ ID NO: 190912.

The probes having the nucleotide sequence represented by SEQ ID NOS: 192309, 192310, 192311, 192312, 192313, 192314, 192315, 192316, 192317, 192318, 192319 were selected from the ORFs having the nucleotide sequence represented by SEQ ID NO: 190913.

The probes having the nucleotide sequence represented by SEQ ID NOS: 192320, 192321, 192322, 192323, 192324, 192325, 192326, 192327, 192328, 192329, 192330 were selected from the ORFs having the nucleotide sequence represented by SEQ ID NO: 190914.

The probes having the nucleotide sequence represented by SEQ ID NOS: 192331, 192332, 192333, 192334, 192335, 192336, 192337, 192338, 192339, 192340, 192341 were selected from the ORFs having the nucleotide sequence represented by SEQ ID NO: 190915.

The probes having the nucleotide sequence represented by SEQ ID NOS: 192342, 192343, 192344, 192345, 192346, 192347, 192348, 192349, 192350, 192351, 192352 were selected from the ORFs having the nucleotide sequence represented by SEQ ID NO: 190916.

The probes having the nucleotide sequence represented by SEQ ID NOS: 192353, 192354, 192355, 192356, 192357, 192358, 192359, 192360 were selected from the ORFs having the nucleotide sequence represented by SEQ ID NO: 190917.

The probes having the nucleotide sequence represented by SEQ ID NOS: 192361, 192362, 192363, 192364, 192365, 192366, 192367, 192368, 192369, 192370, 192371 were selected from the ORFs having the nucleotide sequence represented by SEQ, ID NO: 190918.

The probes having the nucleotide sequence represented by SEQ ID NOS: 192372, 192373, 192374, 192375, 192376, 192377, 192378, 192379, 192380, 192381, 192382 were selected from the ORFs having the nucleotide sequence represented by SEQ ID NO: 190919.

The probes having the nucleotide sequence represented by SEQ ID NOS: 192383, 192384, 192385, 192386, 192387, 192388, 192389, 192390, 192391, 192392, 192393 were selected from the ORFs having the nucleotide sequence represented by SEQ ID NO: 190920.

The probes having the nucleotide sequence represented by SEQ ID NOS: 192394, 192395, 192396, 192397, 192398, 192399, 192400, 192401, 192402, 192403, 192404 were selected from the ORFs having the nucleotide sequence represented by SEQ ID NO: 190921.

The probes having the nucleotide sequence represented by SEQ ID NOS: 192405, 192406, 192407, 192408, 192409, 192410, 192411, 192412, 192413, 192414, 192415 were selected from the ORFs having the nucleotide sequence represented by SEQ ID NO: 190922.

The probes having the nucleotide sequence represented by SEQ ID NOS: 192416, 192417, 192418, 192419, 192420, 192421, 192422, 192423, 192424, 192425, 192426 were selected from the ORFs having the nucleotide sequence represented by SEQ ID NO: 190923.

The probes having the nucleotide sequence represented by SEQ ID NOS: 192427, 192428, 192429, 192430, 192431, 192432, 192433, 192434, 192435, 192436, 192437 were selected from the ORFs having the nucleotide sequence represented by SEQ ID NO: 190924.

The probes having the nucleotide sequence represented by SEQ ID NOS: 192438, 192439, 192440, 192441, 192442, 192443, 192444, 192445, 192446, 192447, 192448 were selected from the ORFs having the nucleotide sequence represented by SEQ ID NO: 190925.

The probes having the nucleotide sequence represented by SEQ ID NOS: 192449, 192450, 192451, 192452, 192453, 192454, 192455, 192456, 192457, 192458, 192459 were selected from the ORFs having the nucleotide sequence represented by SEQ ID NO: 190926.

The probes having the nucleotide sequence represented by SEQ ID NOS: 192460, 192461, 192462, 192463, 192464, 192465, 192466, 192467, 192468, 192469, 192470 were selected from the ORFs having the nucleotide sequence represented by SEQ ID NO: 190927.

The probes having the nucleotide sequence represented by SEQ ID NOS: 192471, 192472, 192473, 192474, 192475, 192476, 192477, 192478, 192479, 192480, 192481 were selected from the ORFs having the nucleotide sequence represented by SEQ ID NO: 190928.

The probes having the nucleotide sequence represented by SEQ ID NOS: 192482, 192483, 192484, 192485, 192486, 192487, 192488, 192489, 192490, 192491, 192492 were selected from the ORFs having the nucleotide sequence represented by SEQ ID NO: 190929.

The probes having the nucleotide sequence represented by SEQ ID NOS: 192493, 192494, 192495, 192496, 192497, 192498, 192499, 192500, 192501, 192502, 192503 were selected from the ORFs having the nucleotide sequence represented by SEQ ID NO: 190930.

The probes having the nucleotide sequence represented by SEQ ID NOS: 192504, 192505, 192506, 192507, 192508, 192509, 192510, 192511, 192512, 192513, 192514 were selected from the ORFs having the nucleotide sequence represented by SEQ ID NO: 190931.

The probes having the nucleotide sequence represented by SEQ ID NOS: 192515, 192516, 192517, 192518, 192519, 192520, 192521, 192522, 192523, 192524, 192525 were selected from the ORFs having the nucleotide sequence represented by SEQ ID NO: 190932.

The probes having the nucleotide sequence represented by SEQ ID NOS: 192526, 192527, 192528, 192529, 192530, 192531, 192532, 192533, 192534, 192535 were selected from the ORFs having the nucleotide sequence represented by SEQ ID NO: 190933.

The probes having the nucleotide sequence represented by SEQ ID NOS: 192536, 192537, 192538, 192539, 192540, 192541, 192542, 192543, 192544, 192545, 192546 were selected from the ORFs having the nucleotide sequence represented by SEQ ID NO: 190934.

The probes having the nucleotide sequence represented by SEQ ID NOS: 192547, 192548, 192549, 192550, 192551, 192552, 192553, 192554, 192555, 192556, 192557 were selected from the ORFs having the nucleotide sequence represented by SEQ ID NO: 190935.

The probes having the nucleotide sequence represented by SEQ ID NOS: 192558, 192559, 192560, 192561, 192562, 192563, 192564, 192565, 192566, 192567, 192568 were selected from the ORFs having the nucleotide sequence represented by SEQ ID NO: 190936.

The probes having the nucleotide sequence represented by SEQ ID NOS: 192569, 192570, 192571, 192572, 192573, 192574, 192575, 192576, 192577, 192578, 192579 were selected from the ORFs having the nucleotide sequence represented by SEQ ID NO: 190937.

The probes having the nucleotide sequence represented by SEQ ID NOS: 192580, 192581, 192582, 192583, 192584, 192585, 192586, 192587, 192588, 192589, 192590 were selected from the ORFs having the nucleotide sequence represented by SEQ ID NO: 190938.

The probes having the nucleotide sequence represented by SEQ ID NOS: 192591, 192592, 192593, 192594, 192595, 192596, 192597, 192598, 192599, 192600, 192601 were selected from the ORFs having the nucleotide sequence represented by SEQ ID NO: 190939.

The probes having the nucleotide sequence represented by SEQ ID NOS: 192602, 192603, 192604, 192605, 192606, 192607, 192608, 192609, 192610, 192611, 192612 were selected from the ORFs having the nucleotide sequence represented by SEQ ID NO: 190940.

The probes having the nucleotide sequence represented by SEQ ID NOS: 192613, 192614, 192615, 192616, 192617, 192618, 192619, 192620, 192621, 192622, 192623 were selected from the ORFs having the nucleotide sequence represented by SEQ ID NO: 190941.

The probes having the nucleotide sequence represented by SEQ ID NOS: 192624, 192625, 192626, 192627, 192628, 192629, 192630, 192631, 192632, 192633, 192634 were selected from the ORFs having the nucleotide sequence represented by SEQ ID NO: 190942.

The probes having the nucleotide sequence represented by SEQ ID NOS: 192635, 192636, 192637, 192638, 192639, 192640, 192641, 192642, 192643, 192644, 192645 were selected from the ORFs having the nucleotide sequence represented by SEQ ID NO: 190943.

The probes having the nucleotide sequence represented by SEQ ID NOS: 192646, 192647, 192648, 192649, 192650, 192651, 192652, 192653 were selected from the ORFs having the nucleotide sequence represented by SEQ ID NO: 190944.

The probes having the nucleotide sequence represented by SEQ ID NOS: 192654, 192655, 192656, 192657, 192658, 192659, 192660, 192661, 192662, 192663, 192664 were selected from the ORFs having the nucleotide sequence represented by SEQ ID NO: 190945.

The probes having the nucleotide sequence represented by SEQ ID NOS: 192665, 192666, 192667, 192668, 192669, 192670, 192671, 192672, 192673, 192674, 192675 were selected from the ORFs having the nucleotide sequence represented by SEQ ID NO: 190946.

The probes having the nucleotide sequence represented by SEQ ID NOS: 192676, 192677, 192678, 192679, 192680, 192681, 192682, 192683, 192684, 192685, 192686 were selected from the ORFs having the nucleotide sequence represented by SEQ ID NO: 190947.

The probes having the nucleotide sequence represented by SEQ ID NOS: 192687, 192688, 192689, 192690, 192691, 192692, 192693, 192694, 192695, 192696, 192697 were selected from the ORFs having the nucleotide sequence represented by SEQ ID NO: 190948.

The probes having the nucleotide sequence represented by SEQ ID NOS: 192698, 192699, 192700, 192701, 192702, 192703, 192704, 192705, 192706, 192707, 192708 were selected from the ORFs having the nucleotide sequence represented by SEQ ID NO: 190949.

The probes having the nucleotide sequence represented by SEQ ID NOS: 192709, 192710, 192711, 192712, 192713, 192714, 192715, 192716, 192717, 192718, 192719 were selected from the ORFs having the nucleotide sequence represented by SEQ ID NO: 190950.

The probes having the nucleotide sequence represented by SEQ ID NOS: 192720, 192721, 192722, 192723, 192724, 192725, 192726, 192727 were selected from the ORFs having the nucleotide sequence represented by SEQ ID NO: 190951.

The probes having the nucleotide sequence represented by SEQ ID NOS: 192728, 192729, 192730, 192731, 192732, 192733, 192734, 192735, 192736, 192737, 192738 were selected from the ORFs having the nucleotide sequence represented by SEQ ID NO: 190952.

The probes having the nucleotide sequence represented by SEQ ID NOS: 192739, 192740, 192741, 192742, 192743, 192744, 192745, 192746, 192747, 192748, 192749 were selected from the ORFs having the nucleotide sequence represented by SEQ ID NO: 190953.

The probes having the nucleotide sequence represented by SEQ ID NOS: 192750, 192751, 192752, 192753, 192754, 192755, 192756, 192757, 192758, 192759, 192760 were selected from the ORFs having the nucleotide sequence represented by SEQ ID NO: 190954.

The probes having the nucleotide sequence represented by SEQ ID NOS: 192761, 192762, 192763, 192764, 192765, 192766, 192767, 192768, 192769, 192770, 192771 were selected from the ORFs having the nucleotide sequence represented by SEQ ID NO: 190955.

The probes having the nucleotide sequence represented by SEQ ID NOS: 192772, 192773, 192774, 192775, 192776, 192777, 192778, 192779, 192780, 192781, 192782 were selected from the ORFs having the nucleotide sequence represented by SEQ ID NO: 190956.

The probes having the nucleotide sequence represented by SEQ ID NOS: 192783, 192784, 192785, 192786, 192787, 192788, 192789, 192790, 192791, 192792, 192793 were selected from the ORFs having the nucleotide sequence represented by SEQ ID NO: 190957.

The probes having the nucleotide sequence represented by SEQ ID NOS: 192794, 192795, 192796, 192797, 192798, 192799, 192800, 192801, 192802, 192803, 192804 were selected from the ORFs having the nucleotide sequence represented by SEQ ID NO: 190958.

The probes having the nucleotide sequence represented by SEQ ID NOS: 192805, 192806, 192807, 192808, 192809, 192810, 192811, 192812, 192813, 192814, 192815 were selected from the ORFs having the nucleotide sequence represented by SEQ ID NO: 190959.

The probes having the nucleotide sequence represented by SEQ ID NOS: 192816, 192817, 192818, 192819, 192820, 192821, 192822, 192823, 192824, 192825, 192826 were selected from the ORFs having the nucleotide sequence represented by SEQ ID NO: 190960.

The probes having the nucleotide sequence represented by SEQ ID NOS: 192827, 192828, 192829, 192830, 192831, 192832, 192833, 192834, 192835, 192836, 192837 were selected from the ORFs having the nucleotide sequence represented by SEQ ID NO: 190961.

The probes having the nucleotide sequence represented by SEQ ID NOS: 192838, 192839, 192840, 192841, 192842, 192843, 192844, 192845, 192846, 192847, 192848 were selected from the ORFs having the nucleotide sequence represented by SEQ ID NO: 190962.

The probes having the nucleotide sequence represented by SEQ ID NOS: 192849, 192850, 192851, 192852, 192853, 192854, 192855, 192856, 192857, 192858, 192859 were selected from the ORFs having the nucleotide sequence represented by SEQ ID NO: 190963.

The probes having the nucleotide sequence represented by SEQ ID NOS: 192860, 192861, 192862, 192863, 192864, 192865, 192866, 192867, 192868, 192869, 192870 were selected from the ORFs having the nucleotide sequence represented by SEQ ID NO: 190964.

The probes having the nucleotide sequence represented by SEQ ID NOS: 192871, 192872, 192873, 192874, 192875, 192876, 192877, 192878, 192879, 192880, 192881 were selected from the ORFs having the nucleotide sequence represented by SEQ ID NO: 190965.

The probes having the nucleotide sequence represented by SEQ ID NOS: 192882, 192883, 192884, 192885, 192886, 192887, 192888, 192889, 192890, 192891, 192892 were selected from the ORFs having the nucleotide sequence represented by SEQ ID NO: 190966.

The probes having the nucleotide sequence represented by SEQ ID NOS: 192893, 192894, 192895, 192896, 192897, 192898, 192899, 192900, 192901, 192902, 192903 were selected from the ORFs having the nucleotide sequence represented by SEQ ID NO: 190967.

The probes having the nucleotide sequence represented by SEQ ID NOS: 192904, 192905, 192906, 192907, 192908, 192909, 192910, 192911, 192912, 192913, 192914 were selected from the ORFs having the nucleotide sequence represented by SEQ ID NO: 190968.

The probes having the nucleotide sequence represented by SEQ ID NOS: 192915, 192916, 192917, 192918, 192919, 192920, 192921, 192922, 192923, 192924, 192925 were selected from the ORFs having the nucleotide sequence represented by SEQ ID NO: 190969.

The probes having the nucleotide sequence represented by SEQ ID NOS: 192926, 192927, 192928, 192929, 192930, 192931, 192932, 192933, 192934, 192935, 192936 were selected from the ORFs having the nucleotide sequence represented by SEQ ID NO: 190970.

The probes having the nucleotide sequence represented by SEQ ID NOS: 192937, 192938, 192939, 192940, 192941, 192942, 192943, 192944, 192945, 192946, 192947 were selected from the ORFs having the nucleotide sequence represented by SEQ ID NO: 190971.

The probes having the nucleotide sequence represented by SEQ ID NOS: 192948, 192949, 192950, 192951, 192952, 192953, 192954, 192955, 192956, 192957, 192958 were selected from the ORFs having the nucleotide sequence represented by SEQ ID NO: 190972.

The probes having the nucleotide sequence represented by SEQ ID NOS: 192959, 192960, 192961, 192962, 192963, 192964, 192965, 192966, 192967, 192968, 192969 were selected from the ORFs having the nucleotide sequence represented by SEQ ID NO: 190973.

The probes having the nucleotide sequence represented by SEQ ID NOS: 192970, 192971, 192972, 192973, 192974, 192975, 192976, 192977, 192978, 192979, 192980 were selected from the ORFs having the nucleotide sequence represented by SEQ ID NO: 190974.

The probes having the nucleotide sequence represented by SEQ ID NOS: 192981, 192982, 192983, 192984, 192985, 192986, 192987, 192988, 192989, 192990, 192991 were selected from the ORFs having the nucleotide sequence represented by SEQ ID NO: 190975.

The probes having the nucleotide sequence represented by SEQ ID NOS: 192992, 192993, 192994, 192995, 192996, 192997, 192998, 192999, 193000, 193001, 193002 were selected from the ORFs having the nucleotide sequence represented by SEQ ID NO: 190976.

The probes having the nucleotide sequence represented by SEQ ID NOS: 193003, 193004, 193005, 193006, 193007, 193008, 193009, 193010, 193011, 193012, 193013 were selected from the ORFs having the nucleotide sequence represented by SEQ ID NO: 190977.

The probes having the nucleotide sequence represented by SEQ ID NOS: 193014, 193015, 193016, 193017, 193018, 193019, 193020, 193021, 193022, 193023, 193024 were selected from the ORFs having the nucleotide sequence represented by SEQ ID NO: 190978.

The probes having the nucleotide sequence represented by SEQ ID NOS: 193036, 193037, 193038, 193039, 193040, 193041, 193042, 193043, 193044, 193045, 193046 were selected from the ORFs having the nucleotide sequence represented by SEQ ID NO: 190979.

The probes having the nucleotide sequence represented by SEQ ID NOS: 193025, 193026, 193027, 193028, 193029, 193030, 193031, 193032, 193033, 193034, 193035 were selected from the ORFs having the nucleotide sequence represented by SEQ ID NO: 190980.

The probes having the nucleotide sequence represented by SEQ ID NOS: 193047, 193048, 193049, 193050, 193051, 193052, 193053, 193054, 193055, 193056, 193057 were selected from the ORFs having the nucleotide sequence represented by SEQ ID NO: 190981.

The probes having the nucleotide sequence represented by SEQ ID NOS: 193058, 193059, 193060, 193061, 193062, 193063, 193064, 193065, 193066, 193067, 193068 were selected from the ORFs having the nucleotide sequence represented by SEQ ID NO: 190982.

The probes having the nucleotide sequence represented by SEQ ID NOS: 193069, 193070, 193071, 193072, 193073, 193074, 193075, 193076, 193077, 193078, 193079 were selected from the ORFs having the nucleotide sequence represented by SEQ ID NO: 190983.

The probes having the nucleotide sequence represented by SEQ ID NOS: 193080, 193081, 193082, 193083, 193084, 193085, 193086, 193087, 193088, 193089, 193090 were selected from the ORFs having the nucleotide sequence represented by SEQ ID NO: 190984.

The probes having the nucleotide sequence represented by SEQ ID NOS: 193091, 193092, 193093, 193094, 193095, 193096, 193097, 193098, 193099, 193100, 193101 were selected from the ORFs having the nucleotide sequence represented by SEQ ID NO: 190985.

The probes having the nucleotide sequence represented by SEQ ID NOS: 193102, 193103, 193104, 193105, 193106, 193107, 193108, 193109, 193110, 193111, 193112 were selected from the ORFs having the nucleotide sequence represented by SEQ ID NO: 190986.

The probes having the nucleotide sequence represented by SEQ ID NOS: 193113, 193114, 193115, 193116, 193117, 193118, 193119, 193120, 193121, 193122, 193123 were selected from the ORFs having the nucleotide sequence represented by SEQ ID NO: 190987.

The probes having the nucleotide sequence represented by SEQ ID NOS: 193124, 193125, 193126, 193127, 193128, 193129, 193130, 193131, 193132, 193133, 193134 were selected from the ORFs having the nucleotide sequence represented by SEQ ID NO: 190988.

The probes having the nucleotide sequence represented by SEQ ID NOS: 193135, 193136, 193137, 193138, 193139, 193140, 193141, 193142, 193143, 193144, 193145 were selected from the ORFs having the nucleotide sequence represented by SEQ ID NO: 190989.

The probes having the nucleotide sequence represented by SEQ ID NOS: 193146, 193147, 193148, 193149, 193150, 193151, 193152, 193153, 193154, 193155, 193156 were selected from the ORFs having the nucleotide sequence represented by SEQ ID NO: 190990.

The probes having the nucleotide sequence represented by SEQ ID NOS: 193157, 193158, 193159, 193160, 193161, 193162, 193163, 193164, 193165, 193166, 193167 were selected from the ORFs having the nucleotide sequence represented by SEQ ID NO: 190991.

The probes having the nucleotide sequence represented by SEQ ID NOS: 193168, 193169, 193170, 193171, 193172, 193173, 193174, 193175, 193176, 193177, 193178 were selected from the ORFs having the nucleotide sequence represented by SEQ ID NO: 190992.

The probes having the nucleotide sequence represented by SEQ ID NOS: 193179, 193180, 193181, 193182, 193183, 193184, 193185, 193186, 193187, 193188, 193189 were selected from the ORFs having the nucleotide sequence represented by SEQ ID NO: 190993.

The probes having the nucleotide sequence represented by SEQ ID NOS: 193190, 193191, 193192, 193193, 193194, 193195, 193196, 193197, 193198, 193199, 193200 were selected from the ORFs having the nucleotide sequence represented by SEQ ID NO: 190994.

The probes having the nucleotide sequence represented by SEQ ID NOS: 193201, 193202, 193203, 193204, 193205, 193206, 193207, 193208, 193209, 193210, 193211 were selected from the ORFs having the nucleotide sequence represented by SEQ ID NO: 190995.

The probes having the nucleotide sequence represented by SEQ ID NOS: 193212, 193213, 193214, 193215, 193216, 193217, 193218, 193219, 193220, 193221, 193222 were selected from the ORFs having the nucleotide sequence represented by SEQ ID NO: 190996.

The probes having the nucleotide sequence represented by SEQ ID NOS: 193223, 193224, 193225, 193226, 193227, 193228, 193229, 193230, 193231, 193232, 193233 were selected from the ORFs having the nucleotide sequence represented by SEQ ID NO: 190997.

The probes having the nucleotide sequence represented by SEQ ID NOS: 193234, 193235, 193236, 193237, 193238, 193239, 193240, 193241, 193242, 193243, 193244 were selected from the ORFs having the nucleotide sequence represented by SEQ ID NO: 190998.

The probes having the nucleotide sequence represented by SEQ ID NOS: 193245, 193246, 193247, 193248, 193249, 193250, 193251, 193252, 193253, 193254, 193255 were selected from the ORFs having the nucleotide sequence represented by SEQ ID NO: 190999.

The probes having the nucleotide sequence represented by SEQ ID NOS: 193256, 193257, 193258, 193259, 193260, 193261, 193262, 193263, 193264, 193265, 193266 were selected from the ORFs having the nucleotide sequence represented by SEQ ID NO: 191000.

The probes having the nucleotide sequence represented by SEQ ID NOS: 193267, 193268, 193269, 193270, 193271, 193272, 193273, 193274, 193275, 193276, 193277 were selected from the ORFs having the nucleotide sequence represented by SEQ ID NO: 191001.

The probes having the nucleotide sequence represented by SEQ ID NOS: 193278, 193279, 193280, 193281, 193282, 193283, 193284, 193285, 193286, 193287, 193288 were selected from the ORFs having the nucleotide sequence represented by SEQ ID NO: 191002.

The probes having the nucleotide sequence represented by SEQ ID NOS: 193289, 193290, 193291, 193292, 193293, 193294, 193295, 193296, 193297, 193298, 193299 were selected from the ORFs having the nucleotide sequence represented by SEQ ID NO: 191003.

The probes having the nucleotide sequence represented by SEQ ID NOS: 193300, 193301, 193302, 193303, 193304, 193305, 193306, 193307, 193308, 193309, 193310 were selected from the ORFs having the nucleotide sequence represented by SEQ ID NO: 191004.

The probes having the nucleotide sequence represented by SEQ ID NOS: 193311, 193312, 193313, 193314, 193315, 193316, 193317, 193318, 193319, 193320, 193321 were selected from the ORFs having the nucleotide sequence represented by SEQ ID NO: 191005.

The probes having the nucleotide sequence represented by SEQ ID NOS: 193322, 193323, 193324, 193325, 193326, 193327, 193328, 193329, 193330, 193331, 193332 were selected from the ORFs having the nucleotide sequence represented by SEQ ID NO: 191006.

The probes having the nucleotide sequence represented by SEQ ID NOS: 193333, 193334, 193335, 193336, 193337, 193338, 193339, 193340, 193341, 193342, 193343 were selected from the ORFs having the nucleotide sequence represented by SEQ ID NO: 191007.

The probes having the nucleotide sequence represented by SEQ ID NOS: 193344, 193345, 193346, 193347, 193348, 193349, 193350, 193351, 193352, 193353, 193354 were selected from the ORFs having the nucleotide sequence represented by SEQ ID NO: 191008.

The probes having the nucleotide sequence represented by SEQ ID NOS: 193355, 193356, 193357, 193358, 193359, 193360, 193361, 193362, 193363, 193364, 193365 were selected from the ORFs having the nucleotide sequence represented by SEQ ID NO: 191009.

The probes having the nucleotide sequence represented by SEQ ID NOS: 193366, 193367, 193368, 193369, 193370, 193371, 193372, 193373, 193374, 193375, 193376 were selected from the ORFs having the nucleotide sequence represented by SEQ ID NO: 191010.

The probes having the nucleotide sequence represented by SEQ ID NOS: 193377, 193378, 193379, 193380, 193381, 193382, 193383, 193384, 193385, 193386, 193387 were selected from the ORFs having the nucleotide sequence represented by SEQ ID NO: 191011.

The probes having the nucleotide sequence represented by SEQ ID NOS: 193388, 193389, 193390, 193391, 193392, 193393, 193394, 193395, 193396, 193397, 193398 were selected from the ORFs having the nucleotide sequence represented by SEQ ID NO: 191012.

The probes having the nucleotide sequence represented by SEQ ID NOS: 193399, 193400, 193401, 193402, 193403, 193404, 193405, 193406, 193407, 193408, 193409 were selected from the ORFs having the nucleotide sequence represented by SEQ ID NO: 191013.

The probes having the nucleotide sequence represented by SEQ ID NOS: 193410, 193411, 193412, 193413, 193414, 193415, 193416, 193417, 193418, 193419, 193420 were selected from the ORFs having the nucleotide sequence represented by SEQ ID NO: 191014.

The probes having the nucleotide sequence represented by SEQ ID NOS: 193421, 193422, 193423, 193424, 193425, 193426, 193427, 193428, 193429, 193430, 193431 were selected from the ORFs having the nucleotide sequence represented by SEQ ID NO: 191015.

The probes having the nucleotide sequence represented by SEQ ID NOS: 193432, 193433, 193434, 193435, 193436, 193437, 193438, 193439, 193440, 193441, 193442 were selected from the ORFs having the nucleotide sequence represented by SEQ ID NO: 191016.

The probes having the nucleotide sequence represented by SEQ ID NOS: 193443, 193444, 193445, 193446, 193447, 193448, 193449, 193450, 193451, 193452, 193453 were selected from the ORFs having the nucleotide sequence represented by SEQ ID NO: 191017.

The probes having the nucleotide sequence represented by SEQ ID NOS: 193454, 193455, 193456, 193457, 193458, 193459, 193460, 193461, 193462, 193463, 193464 were selected from the ORFs having the nucleotide sequence represented by SEQ ID NO: 191018.

The probes having the nucleotide sequence represented by SEQ ID NOS: 193465, 193466, 193467, 193468, 193469, 193470, 193471, 193472, 193473, 193474, 193475 were selected from the ORFs having the nucleotide sequence represented by SEQ ID NO: 191019.

The probes having the nucleotide sequence represented by SEQ ID NOS: 193476, 193477, 193478, 193479, 193480, 193481, 193482, 193483, 193484, 193485, 193486 were selected from the ORFs having the nucleotide sequence represented by SEQ ID NO: 191020.

The probes having the nucleotide sequence represented by SEQ ID NOS: 193487, 193488, 193489, 193490, 193491, 193492, 193493, 193494, 193495, 193496, 193497 were selected from the ORFs having the nucleotide sequence represented by SEQ ID NO: 191021.

The probes having the nucleotide sequence represented by SEQ ID NOS: 193498, 193499, 193500, 193501, 193502, 193503, 193504, 193505, 193506, 193507, 193508 were selected from the ORFs having the nucleotide sequence represented by SEQ ID NO: 191022.

The probes having the nucleotide sequence represented by SEQ ID NOS: 193509, 193510, 193511, 193512, 193513, 193514, 193515, 193516, 193517, 193518, 193519 were selected from the ORFs having the nucleotide sequence represented by SEQ ID NO: 191023.

The probes having the nucleotide sequence represented by SEQ ID NOS: 193520, 193521, 193522, 193523, 193524, 193525, 193526, 193527, 193528, 193529, 193530 were selected from the ORFs having the nucleotide sequence represented by SEQ ID NO: 191024.

The probes having the nucleotide sequence represented by SEQ ID NOS: 193531, 193532, 193533, 193534, 193535, 193536, 193537, 193538, 193539, 193540, 193541 were selected from the ORFs having the nucleotide sequence represented by SEQ ID NO: 191025.

The probes having the nucleotide sequence represented by SEQ ID NOS: 193542, 193543, 193544, 193545, 193546, 193547, 193548, 193549, 193550, 193551, 193552 were selected from the ORFs having the nucleotide sequence represented by SEQ ID NO: 191026.

The probes having the nucleotide sequence represented by SEQ ID NOS: 193553, 193554, 193555, 193556, 193557, 193558, 193559, 193560, 193561, 193562, 193563 were selected from the ORFs having the nucleotide sequence represented by SEQ ID NO: 191027.

The probes having the nucleotide sequence represented by SEQ ID NOS: 193564, 193565, 193566, 193567, 193568, 193569, 193570, 193571, 193572, 193573, 193574 were selected from the ORFs having the nucleotide sequence represented by SEQ ID NO: 191028.

The probes having the nucleotide sequence represented by SEQ ID NOS: 193575, 193576, 193577, 193578, 193579, 193580, 193581, 193582, 193583, 193584, 193585 were selected from the ORFs having the nucleotide sequence represented by SEQ ID NO: 191029.

The probes having the nucleotide sequence represented by SEQ ID NOS: 193586, 193587, 193588, 193589, 193590, 193591, 193592, 193593, 193594, 193595, 193596 were selected from the ORFs having the nucleotide sequence represented by SEQ ID NO: 191030.

The probes having the nucleotide sequence represented by SEQ ID NOS: 193597, 193598, 193599, 193600, 193601, 193602, 193603, 193604, 193605, 193606, 193607 were selected from the ORFs having the nucleotide sequence represented by SEQ ID NO: 191031.

The probes having the nucleotide sequence represented by SEQ ID NOS: 193608, 193609, 193610, 193611, 193612, 193613, 193614, 193615, 193616, 193617, 193618 were selected from the ORFs having the nucleotide sequence represented by SEQ ID NO: 191032.

The probes having the nucleotide sequence represented by SEQ ID NOS: 193619, 193620, 193621, 193622, 193623, 193624, 193625, 193626, 193627, 193628, 193629 were selected from the ORFs having the nucleotide sequence represented by SEQ ID NO: 191033.

The probes having the nucleotide sequence represented by SEQ ID NOS: 193630, 193631, 193632, 193633, 193634, 193635, 193636, 193637, 193638, 193639, 193640 were selected from the ORFs having the nucleotide sequence represented by SEQ ID NO: 191034.

The probes having the nucleotide sequence represented by SEQ ID NOS: 193641, 193642, 193643, 193644, 193645, 193646, 193647, 193648, 193649, 193650, 193651 were selected from the ORFs having the nucleotide sequence represented by SEQ ID NO: 191035.

The probes having the nucleotide sequence represented by SEQ ID NOS: 193652, 193653, 193654, 193655, 193656, 193657, 193658, 193659, 193660, 193661, 193662 were selected from the ORFs having the nucleotide sequence represented by SEQ ID NO: 191036.

The probes having the nucleotide sequence represented by SEQ ID NOS: 193663, 193664, 193665, 193666, 193667, 193668, 193669, 193670, 193671, 193672, 193673 were selected from the ORFs having the nucleotide sequence represented by SEQ ID NO: 191037.

The probes having the nucleotide sequence represented by SEQ ID NOS: 193674, 193675, 193676, 193677, 193678, 193679, 193680, 193681, 193682, 193683, 193684 were selected from the ORFs having the nucleotide sequence represented by SEQ ID NO: 191038.

The probes having the nucleotide sequence represented by SEQ ID NOS: 193685, 193686, 193687, 193688, 193689, 193690, 193691, 193692, 193693, 193694, 193695 were selected from the ORFs having the nucleotide sequence represented by SEQ ID NO: 191039.

The probes having the nucleotide sequence represented by SEQ ID NOS: 193696, 193697, 193698, 193699, 193700, 193701, 193702, 193703, 193704, 193705, 193706 were selected from the ORFs having the nucleotide sequence represented by SEQ ID NO: 191040.

The probes having the nucleotide sequence represented by SEQ ID NOS: 193707, 193708, 193709, 193710, 193711, 193712, 193713, 193714, 193715, 193716, 193717 were selected from the ORFs having the nucleotide sequence represented by SEQ ID NO: 191041.

The probes having the nucleotide sequence represented by SEQ ID NOS: 193718, 193719, 193720, 193721, 193722, 193723, 193724, 193725, 193726, 193727, 193728 were selected from the ORFs having the nucleotide sequence represented by SEQ ID NO: 191042.

The probes having the nucleotide sequence represented by SEQ ID NOS: 193729, 193730, 193731, 193732, 193733, 193734, 193735, 193736, 193737, 193738, 193739 were selected from the ORFs having the nucleotide sequence represented by SEQ ID NO: 191043.

The probes having the nucleotide sequence represented by SEQ ID NOS: 193740, 193741, 193742, 193743, 193744, 193745, 193746, 193747, 193748, 193749, 193750 were selected from the ORFs having the nucleotide sequence represented by SEQ ID NO: 191044.

The probes having the nucleotide sequence represented by SEQ ID NOS: 193751, 193752, 193753, 193754, 193755, 193756, 193757, 193758, 193759, 193760, 193761 were selected from the ORFs having the nucleotide sequence represented by SEQ ID NO: 191045.

The probes having the nucleotide sequence represented by SEQ ID NOS: 193762, 193763, 193764, 193765, 193766, 193767, 193768, 193769, 193770, 193771, 193772 were selected from the ORFs having the nucleotide sequence represented by SEQ ID NO: 191046.

The probes having the nucleotide sequence represented by SEQ ID NOS: 193773, 193774, 193775, 193776, 193777, 193778, 193779, 193780, 193781, 193782, 193783 were selected from the ORFs having the nucleotide sequence represented by SEQ ID NO: 191047.

The probes having the nucleotide sequence represented by SEQ ID NOS: 193784, 193785, 193786, 193787, 193788, 193789, 193790, 193791, 193792, 193793, 193794 were selected from the ORFs having the nucleotide sequence represented by SEQ ID NO: 191048.

The probes having the nucleotide sequence represented by SEQ ID NOS: 193795, 193796, 193797, 193798, 193799, 193800, 193801, 193802, 193803, 193804, 193805 were selected from the ORFs having the nucleotide sequence represented by SEQ ID NO: 191049.

The probes having the nucleotide sequence represented by SEQ ID NOS: 193806, 193807, 193808, 193809, 193810, 193811, 193812, 193813, 193814, 193815, 193816 were selected from the ORFs having the nucleotide sequence represented by SEQ ID NO: 191050.

The probes having the nucleotide sequence represented by SEQ ID NOS: 193817, 193818, 193819, 193820, 193821, 193822, 193823, 193824, 193825, 193826, 193827 were selected from the ORFs having the nucleotide sequence represented by SEQ ID NO: 191051.

The probes having the nucleotide sequence represented by SEQ ID NOS: 193828, 193829, 193830, 193831, 193832, 193833, 193834, 193835, 193836, 193837, 193838 were selected from the ORFs having the nucleotide sequence represented by SEQ ID NO: 191052.

The probes having the nucleotide sequence represented by SEQ ID NOS: 193839, 193840, 193841, 193842, 193843, 193844, 193845, 193846, 193847, 193848, 193849 were selected from the ORFs having the nucleotide sequence represented by SEQ ID NO: 191053.

The probes having the nucleotide sequence represented by SEQ ID NOS: 193850, 193851, 193852, 193853, 193854, 193855, 193856, 193857, 193858, 193859, 193860 were selected from the ORFs having the nucleotide sequence represented by SEQ ID NO: 191054.

The probes having the nucleotide sequence represented by SEQ ID NOS: 193861, 193862, 193863, 193864, 193865, 193866, 193867, 193868, 193869, 193870, 193871 were selected from the ORFs having the nucleotide sequence represented by SEQ ID NO: 191055.

The probes having the nucleotide sequence represented by SEQ ID NOS: 193872, 193873, 193874, 193875, 193876, 193877, 193878, 193879, 193880, 193881, 193882 were selected from the ORFs having the nucleotide sequence represented by SEQ ID NO: 191056.

The probes having the nucleotide sequence represented by SEQ ID NOS: 193883, 193884, 193885, 193886, 193887, 193888, 193889, 193890, 193891, 193892 were selected from the ORFs having the nucleotide sequence represented by SEQ ID NO: 191057.

The probes having the nucleotide sequence represented by SEQ ID NOS: 193893, 193894, 193895, 193896, 193897, 193898, 193899, 193900, 193901, 193902, 193903 were selected from the ORFs having the nucleotide sequence represented by SEQ ID NO: 191058.

The probes having the nucleotide sequence represented by SEQ ID NOS: 193904, 193905, 193906, 193907, 193908, 193909, 193910, 193911, 193912, 193913, 193914 were selected from the ORFs having. the nucleotide sequence represented by SEQ ID NO: 191059.

The probes having the nucleotide sequence represented by SEQ ID NOS: 193915, 193916, 193917, 193918, 193919, 193920, 193921, 193922, 193923, 193924, 193925 were selected from the ORFs having the nucleotide sequence represented by SEQ ID NO: 191060.

The probes having the nucleotide sequence represented by SEQ ID NOS: 193926, 193927, 193928, 193929, 193930, 193931, 193932, 193933, 193934, 193935, 193936 were selected from the ORFs having the nucleotide sequence represented by SEQ ID NO: 191061.

The probes having the nucleotide sequence represented by SEQ ID NOS: 193937, 193938, 193939, 193940, 193941, 193942, 193943, 193944, 193945, 193946, 193947 were selected from the ORFs having the nucleotide sequence represented by SEQ ID NO: 191062.

The probes having the nucleotide sequence represented by SEQ ID NOS: 193948, 193949, 193950, 193951, 193952, 193953, 193954, 193955, 193956, 193957, 193958 were selected from the ORFs having the nucleotide sequence represented by SEQ ID NO: 191063.

The probes having the nucleotide sequence represented by SEQ ID NOS: 193959, 193960, 193961, 193962, 193963, 193964, 193965, 193966, 193967, 193968, 193969 were selected from the ORFs having the nucleotide sequence represented by SEQ ID NO: 191064.

The probes having the nucleotide sequence represented by SEQ ID NOS: 193970, 193971, 193972, 193973, 193974, 193975, 193976, 193977, 193978, 193979, 193980 were selected from the ORFs having the nucleotide sequence represented by SEQ ID NO: 191065.

The probes having the nucleotide sequence represented by SEQ ID NOS: 193981, 193982, 193983, 193984, 193985, 193986, 193987, 193988, 193989, 193990, 193991 were selected from the ORFs having the nucleotide sequence represented by SEQ ID NO: 191066.

The probes having the nucleotide sequence represented by SEQ ID NOS: 193992, 193993, 193994, 193995, 193996, 193997, 193998, 193999, 194000, 194001, 194002 were selected from the ORFs having the nucleotide sequence represented by SEQ ID NO: 191067.

The probes having the nucleotide sequence represented by SEQ ID NOS: 194003, 194004, 194005, 194006, 194007, 194008, 194009, 194010, 194011, 194012, 194013 were selected from the ORFs having the nucleotide sequence represented by SEQ ID NO: 191068.

The probes having the nucleotide sequence represented by SEQ ID NOS: 194014, 194015, 194016, 194017, 194018, 194019, 194020, 194021, 194022, 194023, 194024 were selected from the ORFs having the nucleotide sequence represented by SEQ ID NO: 191069.

The probes having the nucleotide sequence represented by SEQ ID NOS: 194025, 194026, 194027, 194028, 194029, 194030, 194031, 194032, 194033, 194034, 194035 were selected from the ORFs having the nucleotide sequence represented by SEQ ID NO: 191070.

The probes having the nucleotide sequence represented by SEQ ID NOS: 194036, 194037, 194038, 194039, 194040, 194041, 194042, 194043, 194044, 194045, 194046 were selected from the ORFs having the nucleotide sequence represented by SEQ ID NO: 191071.

The probes having the nucleotide sequence represented by SEQ ID NOS: 194047, 194048, 194049, 194050, 194051, 194052, 194053, 194054, 194055, 194056, 194057 were selected from the ORFs having the nucleotide sequence represented by SEQ ID NO: 191072.

The probes having the nucleotide sequence represented by SEQ ID NOS: 194058, 194059, 194060, 194061, 194062, 194063, 194064, 194065, 194066, 194067, 194068 were selected from the ORFs having the nucleotide sequence represented by SEQ ID NO: 191073.

The probes having the nucleotide sequence represented by SEQ ID NOS: 194069, 194070, 194071, 194072, 194073, 194074, 194075, 194076, 194077, 194078, 194079 were selected from the ORFs having the nucleotide sequence represented by SEQ ID NO: 191074.

The probes having the nucleotide sequence represented by SEQ ID NOS: 194080, 194081, 194082, 194083, 194084, 194085, 194086, 194087, 194088, 194089, 194090 were selected from the ORFs having the nucleotide sequence represented by SEQ ID NO: 191075.

The probes having the nucleotide sequence represented by SEQ ID NOS: 194091, 194092, 194093, 194094, 194095, 194096, 194097, 194098, 194099, 194100, 194101 were selected from the ORFs having the nucleotide sequence represented by SEQ ID NO: 191076.

The probes having the nucleotide sequence represented by SEQ ID NOS: 194102, 194103, 194104, 194105, 194106, 194107, 194108, 194109, 194110, 194111, 194112 were selected from the ORFs having the nucleotide sequence represented by SEQ ID NO: 191077.

The probes having the nucleotide sequence represented by SEQ ID NOS: 194113, 194114, 194115, 194116, 194117, 194118, 194119, 194120, 194121, 194122, 194123 were selected from the ORFs having the nucleotide sequence represented by SEQ ID NO: 191078.

The probes having the nucleotide sequence represented by SEQ ID NOS: 194124, 194125, 194126, 194127, 194128, 194129, 194130, 194131, 194132, 194133, 194134 were selected from the ORFs having the nucleotide sequence represented by SEQ ID NO: 191079.

The probes having the nucleotide sequence represented by SEQ ID NOS: 194135, 194136, 194137, 194138, 194139, 194140, 194141, 194142, 194143, 194144, 194145 were selected from the ORFs having the nucleotide sequence represented by SEQ ID NO: 191080.

The probes having the nucleotide sequence represented by SEQ ID NOS: 194146, 194147, 194148, 194149, 194150, 194151, 194152, 194153, 194154, 194155, 194156 were selected from the ORFs having the nucleotide sequence represented by SEQ ID NO: 191081.

The probes having the nucleotide sequence represented by SEQ ID NOS: 194157, 194158, 194159, 194160, 194161, 194162, 194163, 194164 were selected from the ORFs having the nucleotide sequence represented by SEQ ID NO: 191082.

The probes having the nucleotide sequence represented by SEQ ID NOS: 194165, 194166, 194167, 194168, 194169, 194170, 194171, 194172, 194173, 194174, 194175 were selected from the ORFs having the nucleotide sequence represented by SEQ ID NO: 191083.

The probes having the nucleotide sequence represented by SEQ ID NOS: 194176, 194177, 194178, 194179, 194180, 194181, 194182, 194183, 194184, 194185, 194186 were selected from the ORFs having the nucleotide sequence represented by SEQ ID NO: 191084.

The probes having the nucleotide sequence represented by SEQ ID NOS: 194187, 194188, 194189, 194190, 194191, 194192, 194193, 194194, 194195, 194196, 194197 were selected from the ORFs having the nucleotide sequence represented by SEQ ID NO: 191085.

The probes having the nucleotide sequence represented by SEQ ID NOS: 194198, 194199, 194200, 194201, 194202, 194203, 194204, 194205, 194206, 194207, 194208 were selected from the ORFs having the nucleotide sequence represented by SEQ ID NO: 191086.

The probes having the nucleotide sequence represented by SEQ ID NOS: 194209, 194210, 194211, 194212, 194213, 194214, 194215, 194216, 194217, 194218, 194219 were selected from the ORFs having the nucleotide sequence represented by SEQ ID NO: 191087.

The probes having the nucleotide sequence represented by SEQ ID NOS: 194220, 194221, 194222, 194223, 194224, 194225, 194226, 194227, 194228, 194229, 194230 were selected from the ORFs having the nucleotide sequence represented by SEQ ID NO: 191088.

The probes having the nucleotide sequence represented by SEQ ID NOS: 194231, 194232, 194233, 194234, 194235, 194236, 194237, 194238, 194239, 194240, 194241 were selected from the ORFs having the nucleotide sequence represented by SEQ ID NO: 191089.

The probes having the nucleotide sequence represented by SEQ ID NOS: 194242, 194243, 194244, 194245, 194246, 194247, 194248, 194249, 194250, 194251, 194252 were selected from the ORFs having the nucleotide sequence represented by SEQ ID NO: 191090.

The probes having the nucleotide sequence represented by SEQ ID NOS: 194253, 194254, 194255, 194256, 194257, 194258, 194259, 194260, 194261, 194262, 194263 were selected from the ORFs having the nucleotide sequence represented by SEQ ID NO: 191091.

The probes having the nucleotide sequence represented by SEQ ID NOS: 194264, 194265, 194266, 194267, 194268, 194269, 194270, 194271, 194272, 194273, 194274 were selected from the ORFs having the nucleotide sequence represented by SEQ ID NO: 191092.

The probes having the nucleotide sequence represented by SEQ ID NOS: 194275, 194276, 194277, 194278, 194279, 194280, 194281, 194282, 194283, 194284, 194285 were selected from the ORFs having the nucleotide sequence represented by SEQ ID NO: 191093.

The probes having the nucleotide sequence represented by SEQ ID NOS: 194286, 194287, 194288, 194289, 194290, 194291, 194292, 194293, 194294, 194295, 194296 were selected from the ORFs having the nucleotide sequence represented by SEQ ID NO: 191094.

The probes having the nucleotide sequence represented by SEQ ID NOS: 194297, 194298, 194299, 194300, 194301, 194302, 194303, 194304, 194305, 194306, 194307 were selected from the ORFs having the nucleotide sequence represented by SEQ ID NO: 191095.

The probes having the nucleotide sequence represented by SEQ ID NOS: 194308, 194309, 194310, 194311, 194312, 194313, 194314, 194315, 194316, 194317, 194318 were selected from the ORFs having the nucleotide sequence represented by SEQ ID NO: 191096.

The probes having the nucleotide sequence represented by SEQ ID NOS: 194319, 194320, 194321, 194322, 194323, 194324, 194325, 194326, 194327, 194328, 194329 were selected from the ORFs having the nucleotide sequence represented by SEQ ID NO: 191097.

The probes having the nucleotide sequence represented by SEQ ID NOS: 194330, 194331, 194332, 194333, 194334, 194335, 194336, 194337, 194338, 194339, 194340 were selected from the ORFs having the nucleotide sequence represented by SEQ ID NO: 191098.

The probes having the nucleotide sequence represented by SEQ ID NOS: 194341, 194342, 194343, 194344, 194345, 194346, 194347, 194348, 194349, 194350, 194351 were selected from the ORFs having the nucleotide sequence represented by SEQ ID NO: 191099.

The probes having the nucleotide sequence represented by SEQ ID NOS: 194352, 194353, 194354, 194355, 194356, 194357, 194358, 194359, 194360, 194361, 194362 were selected from the ORFs having the nucleotide sequence represented by SEQ ID NO: 191100.

The probes having the nucleotide sequence represented by SEQ ID NOS: 194363, 194364, 194365, 194366, 194367, 194368, 194369, 194370, 194371, 194372, 194373 were selected from the ORFs having the nucleotide sequence represented by SEQ ID NO: 191101.

The probes having the nucleotide sequence represented by SEQ ID NOS: 194374, 194375, 194376, 194377, 194378, 194379, 194380, 194381, 194382, 194383, 194384 were selected from the ORFs having the nucleotide sequence represented by SEQ ID NO: 191102.

The probes having the nucleotide sequence represented by SEQ ID NOS: 194385, 194386, 194387, 194388, 194389, 194390, 194391, 194392, 194393, 194394, 194395 were selected from the ORFs having the nucleotide sequence represented by SEQ ID NO: 191103.

The probes having the nucleotide sequence represented by SEQ ID NOS: 194396, 194397, 194398, 194399, 194400, 194401, 194402, 194403 were selected from the ORFs having the nucleotide sequence represented by SEQ ID NO: 191104.

The probes having the nucleotide sequence represented by SEQ ID NOS: 194404, 194405, 194406, 194407, 194408, 194409, 194410, 194411, 194412, 194413, 194414 were selected from the ORFs having the nucleotide sequence represented by SEQ ID NO: 191105.

The probes having the nucleotide sequence represented by SEQ ID NOS: 194415, 194416, 194417, 194418, 194419, 194420, 194421, 194422, 194423, 194424, 194425 were selected from the ORFs having the nucleotide sequence represented by SEQ ID NO: 191106.

The probes having the nucleotide sequence represented by SEQ ID NOS: 194426, 194427, 194428, 194429, 194430, 194431, 194432, 194433, 194434, 194435, 194436 were selected from the ORFs having the nucleotide sequence represented by SEQ ID NO: 191107.

The probes having the nucleotide sequence represented by SEQ ID NOS: 194437, 194438, 194439, 194440, 194441, 194442, 194443, 194444, 194445, 194446, 194447 were selected from the ORFs having the nucleotide sequence represented by SEQ ID NO: 191108.

The probes having the nucleotide sequence represented by SEQ ID NOS: 194448, 194449, 194450, 194451, 194452, 194453, 194454, 194455, 194456, 194457, 194458 were selected from the ORFs having the nucleotide sequence represented by SEQ ID NO: 191109.

The probes having the nucleotide sequence represented by SEQ ID NOS: 194459, 194460, 194461, 194462, 194463, 194464, 194465, 194466, 194467, 194468, 194469 were selected from the ORFs having the nucleotide sequence represented by SEQ ID NO: 191110.

The probes having the nucleotide sequence represented by SEQ ID NOS: 194470, 194471, 194472, 194473, 194474, 194475, 194476, 194477, 194478, 194479, 194480 were selected from the ORFs having the nucleotide sequence represented by SEQ ID NO: 191111.

The probes having the nucleotide sequence represented by SEQ ID NOS: 194481, 194482, 194483, 194484, 194485, 194486, 194487, 194488 were selected from the ORFs having the nucleotide sequence represented by SEQ ID NO: 191112.

The probes having the nucleotide sequence represented by SEQ ID NOS: 194489, 194490, 194491, 194492, 194493, 194494, 194495, 194496, 194497, 194498, 194499 were selected from the ORFs having the nucleotide sequence represented by SEQ ID NO: 191113.

The probes having the nucleotide sequence represented by SEQ ID NOS: 194500, 194501, 194502, 194503, 194504, 194505, 194506, 194507, 194508, 194509, 194510 were selected from the ORFs having the nucleotide sequence represented by SEQ ID NO: 191114.

The probes having the nucleotide sequence represented by SEQ ID NOS: 194511, 194512, 194513, 194514, 194515, 194516, 194517, 194518, 194519, 194520, 194521 were selected from the ORFs having the nucleotide sequence represented by SEQ ID NO: 191115.

The probes having the nucleotide sequence represented by SEQ ID NOS: 194522, 194523, 194524, 194525, 194526, 194527, 194528, 194529, 194530, 194531, 194532 were selected from the ORFs having the nucleotide sequence represented by SEQ ID NO: 191116.

The probes having the nucleotide sequence represented by SEQ ID NOS: 194533, 194534, 194535, 194536, 194537, 194538, 194539, 194540, 194541, 194542, 194543 were selected from the ORFs having the nucleotide sequence represented by SEQ ID NO: 191117.

The probes having the nucleotide sequence represented by SEQ ID NOS: 194544, 194545, 194546, 194547, 194548, 194549, 194550, 194551, 194552, 194553, 194554 were selected from the ORFs having the nucleotide sequence represented by SEQ ID NO: 191118.

The probes having the nucleotide sequence represented by SEQ ID NOS: 194555, 194556, 194557, 194558, 194559, 194560, 194561, 194562, 194563, 194564, 194565 were selected from the ORFs having the nucleotide sequence represented by SEQ ID NO: 191119.

The probes having the nucleotide sequence represented by SEQ ID NOS: 194566, 194567, 194568, 194569, 194570, 194571, 194572, 194573, 194574, 194575, 194576 were selected from the ORFs having the nucleotide sequence represented by SEQ ID NO: 191120.

The probes having the nucleotide sequence represented by SEQ ID NOS: 194577, 194578, 194579, 194580, 194581, 194582, 194583, 194584, 194585, 194586, 194587 were selected from the ORFs having the nucleotide sequence represented by SEQ ID NO: 191121.

The probes having the nucleotide sequence represented by SEQ ID NOS: 194588, 194589, 194590, 194591, 194592, 194593, 194594, 194595, 194596, 194597, 194598 were selected from the ORFs having the nucleotide sequence represented by SEQ ID NO: 191122.

The probes having the nucleotide sequence represented by SEQ ID NOS: 194599, 194600, 194601, 194602, 194603, 194604, 194605, 194606, 194607, 194608, 194609 were selected from the ORFs having the nucleotide sequence represented by SEQ ID NO: 191123.

The probes having the nucleotide sequence represented by SEQ ID NOS: 194610, 194611, 194612, 194613, 194614, 194615, 194616, 194617, 194618, 194619, 194620 were selected from the ORFs having the nucleotide sequence represented by SEQ ID NO: 191124.

The probes having the nucleotide sequence represented by SEQ ID NOS: 194621, 194622, 194623, 194624, 194625, 194626, 194627, 194628, 194629, 194630, 194631 were selected from the ORFs having the nucleotide sequence represented by SEQ ID NO: 191125.

The probes having the nucleotide sequence represented by SEQ ID NOS: 194632, 194633, 194634, 194635, 194636, 194637, 194638, 194639, 194640, 194641, 194642 were selected from the ORFs having the nucleotide sequence represented by SEQ ID NO: 191126.

The probes having the nucleotide sequence represented by SEQ ID NOS: 194643, 194644, 194645, 194646, 194647, 194648, 194649, 194650 were selected from the ORFs having the nucleotide sequence represented by SEQ ID NO: 191127.

The probes having the nucleotide sequence represented by SEQ ID NOS: 194651, 194652, 194653, 194654, 194655, 194656, 194657, 194658, 194659, 194660, 194661 were selected from the ORFs having the nucleotide sequence represented by SEQ ID NO: 191128.

The probes having the nucleotide sequence represented by SEQ ID NOS: 194662, 194663, 194664, 194665, 194666, 194667, 194668, 194669, 194670, 194671, 194672 were selected from the ORFs having the nucleotide sequence represented by SEQ ID NO: 191129.

The probes having the nucleotide sequence represented by SEQ ID NOS: 194673, 194674, 194675, 194676, 194677, 194678, 194679, 194680, 194681, 194682, 194683 were selected from the ORFs having the nucleotide sequence represented by SEQ ID NO: 191130.

The probes having the nucleotide sequence represented by SEQ ID NOS: 194684, 194685, 194686, 194687, 194688, 194689, 194690, 194691, 194692, 194693, 194694 were selected from the ORFs having the nucleotide sequence represented by SEQ ID NO: 191131.

The probes having the nucleotide sequence represented by SEQ ID NOS: 194695, 194696, 194697, 194698, 194699, 194700, 194701, 194702, 194703, 194704, 194705 were selected from the ORFs having the nucleotide sequence represented by SEQ ID NO: 191132.

The probes having the nucleotide sequence represented by SEQ ID NOS: 194706, 194707, 194708, 194709, 194710, 194711, 194712, 194713, 194714, 194715, 194716 were selected from the ORFs having the nucleotide sequence represented by SEQ ID NO: 191133.

The probes having the nucleotide sequence represented by SEQ ID NOS: 194717, 194718, 194719, 194720, 194721, 194722, 194723, 194724, 194725, 194726, 194727 were selected from the ORFs having the nucleotide sequence represented by SEQ ID NO: 191134.

The probes having the nucleotide sequence represented by SEQ ID NOS: 194728, 194729, 194730, 194731, 194732, 194733, 194734, 194735, 194736, 194737, 194738 were selected from the ORFs having the nucleotide sequence represented by SEQ ID NO: 191135.

The probes having the nucleotide sequence represented by SEQ ID NOS: 194739, 194740, 194741, 194742, 194743, 194744, 194745, 194746, 194747, 194748, 194749 were selected from the ORFs having the nucleotide sequence represented by SEQ ID NO: 191136.

The probes having the nucleotide sequence represented by SEQ ID NOS: 194750, 194751, 194752, 194753, 194754, 194755, 194756, 194757, 194758, 194759, 194760 were selected from the ORFs having the nucleotide sequence represented by SEQ ID NO: 191137.

The probes having the nucleotide sequence represented by SEQ ID NOS: 194761, 194762, 194763, 194764, 194765, 194766, 194767, 194768, 194769, 194770, 194771 were selected from the ORFs having the nucleotide sequence represented by SEQ ID NO: 191138.

The probes having the nucleotide sequence represented by SEQ ID NOS: 194783, 194784, 194785, 194786, 194787, 194788, 194789, 194790, 194791, 194792, 194793 were selected from the ORFs having the nucleotide sequence represented by SEQ ID NO: 191139.

The probes having the nucleotide sequence represented by SEQ ID NOS: 194772, 194773, 194774, 194775, 194776, 194777, 194778, 194779, 194780, 194781, 194782 were selected from the ORFs having the nucleotide sequence represented by SEQ ID NO: 191140.

The probes having the nucleotide sequence represented by SEQ ID NOS: 194794, 194795, 194796, 194797, 194798, 194799, 194800, 194801, 194802, 194803, 194804 were selected from the ORFs having the nucleotide sequence represented by SEQ ID NO: 191141.

The probes having the nucleotide sequence represented by SEQ ID NOS: 194805, 194806, 194807, 194808, 194809, 194810, 194811, 194812, 194813, 194814, 194815 were selected from the ORFs having the nucleotide sequence represented by SEQ ID NO: 191142.

The probes having the nucleotide sequence represented by SEQ ID NOS: 194816, 194817, 194818, 194819, 194820, 194821, 194822, 194823, 194824, 194825, 194826 were selected from the ORFs having the nucleotide sequence represented by SEQ ID NO: 191143.

The probes having the nucleotide sequence represented by SEQ ID NOS: 194827, 194828, 194829, 194830, 194831, 194832, 194833, 194834, 194835, 194836, 194837 were selected from the ORFs having the nucleotide sequence represented by SEQ ID NO: 191144.

The probes having the nucleotide sequence represented by SEQ ID NOS: 194838, 194839, 194840, 194841, 194842, 194843, 194844, 194845, 194846, 194847, 194848 were selected from the ORFs having the nucleotide sequence represented by SEQ ID NO: 191145.

The probes having the nucleotide sequence represented by SEQ ID NOS: 194849, 194850, 194851, 194852, 194853, 194854, 194855, 194856, 194857, 194858, 194859 were selected from the ORFs having the nucleotide sequence represented by SEQ ID NO: 191146.

The probes having the nucleotide sequence represented by SEQ ID NOS: 194860, 194861, 194862, 194863, 194864, 194865, 194866, 194867 were selected from the ORFs having the nucleotide sequence represented by SEQ ID NO: 191147.

The probes having the nucleotide sequence represented by SEQ ID NOS: 194868, 194869, 194870, 194871, 194872, 194873, 194874, 194875, 194876, 194877, 194878 were selected from the ORFs having the nucleotide sequence represented by SEQ ID NO: 191148.

The probes having the nucleotide sequence represented by SEQ ID NOS: 194879, 194880, 194881, 194882, 194883, 194884, 194885, 194886, 194887, 194888, 194889 were selected from the ORFs having the nucleotide sequence represented by SEQ ID NO: 191149.

The probes having the nucleotide sequence represented by SEQ ID NOS: 194890, 194891, 194892, 194893, 194894, 194895, 194896, 194897, 194898, 194899, 194900 were selected from the ORFs having the nucleotide sequence represented by SEQ ID NO: 191150.

The probes having the nucleotide sequence represented by SEQ ID NOS: 194901, 194902, 194903, 194904, 194905, 194906, 194907, 194908 were selected from the ORFs having the nucleotide sequence represented by SEQ ID NO: 191151.

The probes having the nucleotide sequence represented by SEQ ID NOS: 194909, 194910, 194911, 194912, 194913, 194914, 194915, 194916, 194917, 194918, 194919 were selected from the ORFs having the nucleotide sequence represented by SEQ ID NO: 191152.

The probes having the nucleotide sequence represented by SEQ ID NOS: 194920, 194921, 194922, 194923, 194924, 194925, 194926, 194927, 194928, 194929, 194930 were selected from the ORFs having the nucleotide sequence represented by SEQ ID NO: 191153.

The probes having the nucleotide sequence represented by SEQ ID NOS: 194931, 194932, 194933, 194934, 194935, 194936, 194937, 194938, 194939, 194940, 194941 were selected from the ORFs having the nucleotide sequence represented by SEQ ID NO: 191154.

The probes having the nucleotide sequence represented by SEQ ID NOS: 194942, 194943, 194944, 194945, 194946, 194947, 194948, 194949, 194950, 194951, 194952 were selected from the ORFs having the nucleotide sequence represented by SEQ ID NO: 191155.

The probes having the nucleotide sequence represented by SEQ ID NOS: 194953, 194954, 194955, 194956, 194957, 194958, 194959, 194960, 194961, 194962, 194963 were selected from the ORFs having the nucleotide sequence represented by SEQ ID NO: 191156.

The probes having the nucleotide sequence represented by SEQ ID NOS: 194964, 194965, 194966, 194967, 194968, 194969, 194970, 194971, 194972, 194973, 194974 were selected from the ORFs having the nucleotide sequence represented by SEQ ID NO: 191157.

The probes having the nucleotide sequence represented by SEQ ID NOS: 194975, 194976, 194977, 194978, 194979, 194980, 194981, 194982, 194983, 194984, 194985 were selected from the ORFs having the nucleotide sequence represented by SEQ ID NO: 191158.

The probes having the nucleotide sequence represented by SEQ ID NOS: 194986, 194987, 194988, 194989, 194990, 194991, 194992, 194993, 194994, 194995, 194996 were selected from the ORFs having the nucleotide sequence represented by SEQ ID NO: 191159.

The probes having the nucleotide sequence represented by SEQ ID NOS: 194997, 194998, 194999, 195000, 195001, 195002, 195003, 195004, 195005, 195006, 195007 were selected from the ORFs having the nucleotide sequence represented by SEQ ID NO: 191160.

The probes having the nucleotide sequence represented by SEQ ID NOS: 195008, 195009, 195010, 195011, 195012, 195013, 195014, 195015, 195016, 195017, 195018 were selected from the ORFs having the nucleotide sequence represented by SEQ ID NO: 191161.

The probes having the nucleotide sequence represented by SEQ ID NOS: 195019, 195020, 195021, 195022, 195023, 195024, 195025, 195026, 195027, 195028, 195029 were selected from the ORFs having the nucleotide sequence represented by SEQ ID NO: 191162.

The probes having the nucleotide sequence represented by SEQ ID NOS: 195030, 195031, 195032, 195033, 195034, 195035, 195036, 195037, 195038, 195039, 195040 were selected from the ORFs having the nucleotide sequence represented by SEQ ID NO: 191163.

The probes having the nucleotide sequence represented by SEQ ID NOS: 195041, 195042, 195043, 195044, 195045, 195046, 195047, 195048, 195049, 195050, 195051 were selected from the ORFs having the nucleotide sequence represented by SEQ ID NO: 191164.

The probes having the nucleotide sequence represented by SEQ ID NOS: 195052, 195053, 195054, 195055, 195056, 195057, 195058, 195059, 195060, 195061, 195062 were selected from the ORFs having the nucleotide sequence represented by SEQ ID NO: 191165.

The probes having the nucleotide sequence represented by SEQ ID NOS: 195063, 195064, 195065, 195066, 195067, 195068, 195069, 195070, 195071, 195072, 195073 were selected from the ORFs having the nucleotide sequence represented by SEQ ID NO: 191166.

The probes having the nucleotide sequence represented by SEQ ID NOS: 195074, 195075, 195076, 195077, 195078, 195079, 195080, 195081, 195082, 195083, 195084 were selected from the ORFs having the nucleotide sequence represented by SEQ ID NO: 191167.

The probes having the nucleotide sequence represented by SEQ ID NOS: 195085, 195086, 195087, 195088, 195089, 195090, 195091, 195092, 195093, 195094, 195095 were selected from the ORFs having the nucleotide sequence represented by SEQ ID NO: 191168.

The probes having the nucleotide sequence represented by SEQ ID NOS: 195096, 195097, 195098, 195099, 195100, 195101, 195102, 195103, 195104, 195105, 195106 were selected from the ORFs having the nucleotide sequence represented by SEQ ID NO: 191169.

The probes having the nucleotide sequence represented by SEQ ID NOS: 195107, 195108, 195109, 195110, 195111, 195112, 195113, 195114, 195115, 195116, 195117 were selected from the ORFs having the nucleotide sequence represented by SEQ ID NO: 191170.

The probes having the nucleotide sequence represented by SEQ ID NOS: 195118, 195119, 195120, 195121, 195122, 195123, 195124, 195125, 195126, 195127, 195128 were selected from the ORFs having the nucleotide sequence represented by SEQ ID NO: 191171.

The probes having the nucleotide sequence represented by SEQ ID NOS: 195129, 195130, 195131, 195132, 195133, 195134, 195135, 195136, 195137, 195138, 195139 were selected from the ORFs having the nucleotide sequence represented by SEQ ID NO: 191172.

The probes having the nucleotide sequence represented by SEQ ID NOS: 195140, 195141, 195142, 195143, 195144, 195145, 195146, 195147, 195148, 195149, 195150 were selected from the ORFs having the nucleotide sequence represented by SEQ ID NO: 191173.

The probes having the nucleotide sequence represented by SEQ ID NOS: 195151, 195152, 195153, 195154, 195155, 195156, 195157, 195158, 195159, 195160, 195161 were selected from the ORFs having the nucleotide sequence represented by SEQ ID NO: 191174.

The probes having the nucleotide sequence represented by SEQ ID NOS: 195162, 195163, 195164, 195165, 195166, 195167, 195168, 195169, 195170, 195171, 195172 were selected from the ORFs having the nucleotide sequence represented by SEQ ID NO: 191175.

The probes having the nucleotide sequence represented by SEQ ID NOS: 195173, 195174, 195175, 195176, 195177, 195178, 195179, 195180, 195181, 195182, 195183 were selected from the ORFs having the nucleotide sequence represented by SEQ ID NO: 191176.

The probes having the nucleotide sequence represented by SEQ ID NOS: 195184, 195185, 195186, 195187, 195188, 195189, 195190, 195191, 195192, 195193, 195194 were selected from the ORFs having the nucleotide sequence represented by SEQ ID NO: 191177.

The probes having the nucleotide sequence represented by SEQ ID NOS: 195195, 195196, 195197, 195198, 195199, 195200, 195201, 195202, 195203, 195204, 195205 were selected from the ORFs having the nucleotide sequence represented by SEQ ID NO: 191178.

The probes having the nucleotide sequence represented by SEQ ID NOS: 195206, 195207, 195208, 195209, 195210, 195211, 195212, 195213, 195214, 195215, 195216 were selected from the ORFs having the nucleotide sequence represented by SEQ ID NO: 191179.

The probes having the nucleotide sequence represented by SEQ ID NOS: 195217, 195218, 195219, 195220, 195221, 195222, 195223, 195224, 195225, 195226, 195227 were selected from the ORFs having the nucleotide sequence represented by SEQ ID NO: 191180.

The probes having the nucleotide sequence represented by SEQ ID NOS: 195228, 195229, 195230, 195231, 195232, 195233, 195234, 195235 were selected from the ORFs having the nucleotide sequence represented by SEQ ID NO: 191181.

The probes having the nucleotide sequence represented by SEQ ID NOS: 195236, 195237, 195238, 195239, 195240, 195241, 195242, 195243, 195244, 195245, 195246 were selected from the ORFs having the nucleotide sequence represented by SEQ ID NO: 191182.

The probes having the nucleotide sequence represented by SEQ ID NOS: 195247, 195248, 195249, 195250, 195251, 195252, 195253, 195254, 195255, 195256, 195257 were selected from the ORFs having the nucleotide sequence represented by SEQ ID NO: 191183.

The probes having the nucleotide sequence represented by SEQ ID NOS: 195258, 195259, 195260, 195261, 195262, 195263, 195264, 195265, 195266, 195267, 195268 were selected from the ORFs having the nucleotide sequence represented by SEQ ID NO: 191184.

The probes having the nucleotide sequence represented by SEQ ID NOS: 195269, 195270, 195271, 195272, 195273, 195274, 195275, 195276, 195277, 195278, 195279 were selected from the ORFs having the nucleotide sequence represented by SEQ ID NO: 191185.

The probes having the nucleotide sequence represented by SEQ ID NOS: 195280, 195281, 195282, 195283, 195284, 195285, 195286, 195287, 195288, 195289, 195290 were selected from the ORFs having the nucleotide sequence represented by SEQ ID NO: 191186.

The probes having the nucleotide sequence represented by SEQ ID NOS: 195291, 195292, 195293, 195294, 195295, 195296, 195297, 195298, 195299, 195300, 195301 were selected from the ORFs having the nucleotide sequence represented by SEQ ID NO: 191187.

The probes having the nucleotide sequence represented by SEQ ID NOS: 195302, 195303, 195304, 195305, 195306, 195307, 195308, 195309, 195310, 195311, 195312 were selected from the ORFs having the nucleotide sequence represented by SEQ ID NO: 191188.

The probes having the nucleotide sequence represented by SEQ ID NOS: 195313, 195314, 195315, 195316, 195317, 195318, 195319, 195320, 195321, 195322, 195323 were selected from the ORFs having the nucleotide sequence represented by SEQ ID NO: 191189.

The probes having the nucleotide sequence represented by SEQ ID NOS: 195335, 195336, 195337, 195338, 195339, 195340, 195341, 195342, 195343, 195344, 195345 were selected from the ORFs having the nucleotide sequence represented by SEQ ID NO: 191190.

The probes having the nucleotide sequence represented by SEQ ID NOS: 195324, 195325, 195326, 195327, 195328, 195329, 195330, 195331, 195332, 195333, 195334 were selected from the ORFs having the nucleotide sequence represented by SEQ ID NO: 191191.

The probes having the nucleotide sequence represented by SEQ ID NOS: 195346, 195347, 195348, 195349, 195350, 195351, 195352, 195353, 195354, 195355, 195356 were selected from the ORFs having the nucleotide sequence represented by SEQ ID NO: 191192.

The probes having the nucleotide sequence represented by SEQ ID NOS: 195357, 195358, 195359, 195360, 195361, 195362, 195363, 195364, 195365, 195366, 195367 were selected from the ORFs having the nucleotide sequence represented by SEQ ID NO: 191193.

The probes having the nucleotide sequence represented by SEQ ID NOS: 195368, 195369, 195370, 195371, 195372, 195373, 195374, 195375, 195376, 195377, 195378 were selected from the ORFs having the nucleotide sequence represented by SEQ ID NO: 191194.

The probes having the nucleotide sequence represented by SEQ ID NOS: 195379, 195380, 195381, 195382, 195383, 195384, 195385, 195386, 195387, 195388, 195389 were selected from the ORFs having the nucleotide sequence represented by SEQ ID NO: 191195.

The probes having the nucleotide sequence represented by SEQ ID NOS: 195390, 195391, 195392, 195393, 195394, 195395, 195396, 195397, 195398, 195399, 195400 were selected from the ORFs having the nucleotide sequence represented by SEQ ID NO: 191196.

The probes having the nucleotide sequence represented by SEQ ID NOS: 195401, 195402, 195403, 195404, 195405, 195406, 195407, 195408, 195409, 195410, 195411 were selected from the ORFs having the nucleotide sequence represented by SEQ ID NO: 191197.

The probes having the nucleotide sequence represented by SEQ ID NOS: 195412, 195413, 195414, 195415, 195416, 195417, 195418, 195419, 195420, 195421, 195422 were selected from the ORFs having the nucleotide sequence represented by SEQ ID NO: 191198.

The probes having the nucleotide sequence represented by SEQ ID NOS: 195423, 195424, 195425, 195426, 195427, 195428, 195429, 195430, 195431, 195432, 195433 were selected from the ORFs having the nucleotide sequence represented by SEQ ID NO: 191199.

The probes having the nucleotide sequence represented by SEQ ID NOS: 195434, 195435, 195436, 195437, 195438, 195439, 195440, 195441, 195442, 195443, 195444 were selected from the ORFs having the nucleotide sequence represented by SEQ ID NO: 191200.

The probes having the nucleotide sequence represented by SEQ ID NOS: 195445, 195446, 195447, 195448, 195449, 195450, 195451, 195452, 195453, 195454, 195455 were selected from the ORFs having the nucleotide sequence represented by SEQ ID NO: 191201.

The probes having the nucleotide sequence represented by SEQ ID NOS: 195456, 195457, 195458, 195459, 195460, 195461, 195462, 195463, 195464, 195465, 195466 were selected from the ORFs having the nucleotide sequence represented by SEQ ID NO: 191202.

The probes having the nucleotide sequence represented by SEQ ID NOS: 195467, 195468, 195469, 195470, 195471, 195472, 195473, 195474, 195475, 195476, 195477 were selected from the ORFs having the nucleotide sequence represented by SEQ ID NO: 191203.

The probes having the nucleotide sequence represented by SEQ ID NOS: 195478, 195479, 195480, 195481, 195482, 195483, 195484, 195485, 195486, 195487, 195488 were selected from the ORFs having the nucleotide sequence represented by SEQ ID NO: 191204.

The probes having the nucleotide sequence represented by SEQ ID NOS: 195489, 195490, 195491, 195492, 195493, 195494, 195495, 195496, 195497, 195498, 195499 were selected from the ORFs having the nucleotide sequence represented by SEQ ID NO: 191205.

The probes having the nucleotide sequence represented by SEQ ID NOS: 195500, 195501, 195502, 195503, 195504, 195505, 195506, 195507, 195508, 195509, 195510 were selected from the ORFs having the nucleotide sequence represented by SEQ ID NO: 191206.

The probes having the nucleotide sequence represented by SEQ ID NOS: 195511, 195512, 195513, 195514, 195515, 195516, 195517, 195518, 195519, 195520, 195521 were selected from the ORFs having the nucleotide sequence represented by SEQ ID NO: 191207.

The probes having the nucleotide sequence represented by SEQ ID NOS: 195522, 195523, 195524, 195525, 195526, 195527, 195528, 195529, 195530, 195531, 195532 were selected from the ORFs having the nucleotide sequence represented by SEQ ID NO: 191208.

The probes having the nucleotide sequence represented by SEQ ID NOS: 195533, 195534, 195535, 195536, 195537, 195538, 195539, 195540, 195541, 195542, 195543 were selected from the ORFs having the nucleotide sequence represented by SEQ ID NO: 191209.

The probes having the nucleotide sequence represented by SEQ ID NOS: 195544, 195545, 195546, 195547, 195548, 195549, 195550, 195551, 195552, 195553, 195554 were selected from the ORFs having the nucleotide sequence represented by SEQ ID NO: 191210.

The probes having the nucleotide sequence represented by SEQ ID NOS: 195555, 195556, 195557, 195558, 195559, 195560, 195561, 195562, 195563, 195564, 195565 were selected from the ORFs having the nucleotide sequence represented by SEQ ID NO: 191211.

The probes having the nucleotide sequence represented by SEQ ID NOS: 195566, 195567, 195568, 195569, 195570, 195571, 195572, 195573, 195574, 195575, 195576 were selected from the ORFs having the nucleotide sequence represented by SEQ ID NO: 191212.

The probes having the nucleotide sequence represented by SEQ ID NOS: 195577, 195578, 195579, 195580, 195581, 195582, 195583, 195584, 195585, 195586, 195587 were selected from the ORFs having the nucleotide sequence represented by SEQ ID NO: 191213.

The probes from 64 DNA sequences of genes used as internal standard described as above (3) were as follows: The probes having the nucleotide sequence represented by SEQ ID NOS: 195652, 195653, 195654, 195655, 195656, 195657, 195658, 195659, 195660, 195661, 195662, 195663, 195664, 195665, 195666, 195667, 195668, 195669, 195670, 195671, 195672 were selected from the ORFs having the nucleotide sequence represented by SEQ ID NO: 195588.

The probes having the nucleotide sequence represented by SEQ ID NOS: 195673, 195674, 195675, 195676, 195677, 195678, 195679, 195680, 195681, 195682, 195683, 195684, 195685, 195686, 195687, 195688, 195689, 195690, 195691, 195692 were selected from the ORFs having the nucleotide sequence represented by SEQ ID NO: 195589.

The probes having the nucleotide sequence represented by SEQ ID NOS: 195693, 195694, 195695, 195696, 195697, 195698, 195699, 195700, 195701, 195702, 195703, 195704, 195705, 195706, 195707, 195708, 195709, 195710, 195711, 195712, 195713 were selected from the ORFs having the nucleotide sequence represented by SEQ ID NO: 195590.

The probes having the nucleotide sequence represented by SEQ ID NOS: 195714, 195715, 195716, 195717, 195718, 195719, 195720, 195721, 195722, 195723, 195724, 195725, 195726, 195727, 195728, 195729, 195730, 195731, 195732, 195733 were selected from the ORFs having the nucleotide sequence represented by SEQ ID NO: 195591.

The probes having the nucleotide sequence represented by SEQ ID NOS: 195734, 195735, 195736, 195737, 195738, 195739, 195740, 195741, 195742, 195743, 195744, 195745, 195746, 195747, 195748, 195749, 195750, 195751, 195752, 195753 were selected from the ORFs having the nucleotide sequence represented by SEQ ID NO: 195592.

The probes having the nucleotide sequence represented by SEQ ID NOS: 195754, 195755, 195756, 195757, 195758, 195759, 195760, 195761, 195762, 195763, 195764, 195765, 195766, 195767, 195768, 195769, 195770, 195771, 195772, 195773, 195774 were selected from the ORFs having the nucleotide sequence represented by SEQ ID NO: 195593.

The probes having the nucleotide sequence represented by SEQ ID NOS: 195775, 195776, 195777, 195778, 195779, 195780, 195781, 195782, 195783, 195784, 195785, 195786, 195787, 195788, 195789, 195790, 195791, 195792, 195793, 195794 were selected from the ORFs having the nucleotide sequence represented by SEQ ID NO: 195594.

The probes having the nucleotide sequence represented by SEQ ID NOS: 195795, 195796, 195797, 195798, 195799, 195800, 195801, 195802, 195803, 195804, 195805, 195806, 195807, 195808, 195809, 195810, 195811, 195812, 195813, 195814, 195815 were selected from the ORFs having the nucleotide sequence represented by SEQ ID NO: 195595.

The probes having the nucleotide sequence represented by SEQ ID NOS: 195816, 195817, 195818, 195819, 195820, 195821, 195822, 195823, 195824, 195825, 195826, 195827, 195828, 195829, 195830, 195831, 195832, 195833, 195834, 195835 were selected from the ORFs having the nucleotide sequence represented by SEQ ID NO: 195596.

The probes having the nucleotide sequence represented by SEQ ID NOS: 195836, 195837, 195838, 195839, 195840, 195841, 195842, 195843, 195844, 195845, 195846, 195847, 195848, 195849, 195850, 195851, 195852, 195853, 195854, 195855, 195856 were selected from the ORFs having the nucleotide sequence represented by SEQ ID NO: 195597.

The probes having the nucleotide sequence represented by SEQ ID NOS: 195857, 195858, 195859, 195860, 195861, 195862, 95863, 195864, 195865, 195866, 195867, 195868, 195869, 95870, 195871, 195872, 195873, 195874, 195875, 195876 were elected from the ORFs having the nucleotide sequence represented by SEQ ID NO: 195598.

The probes having the nucleotide sequence represented by SEQ ID NOS: 195877, 195878, 195879, 195880, 195881, 195882, 195883, 195884, 195885, 195886, 195887, 195888, 195889, 195890, 195891, 195892, 195893, 195894, 195895, 195896 were selected from the ORFs having the nucleotide sequence represented by SEQ ID NO: 195599.

The probes having the nucleotide sequence represented by SEQ ID NOS: 195897, 195898, 195899, 195900, 195901, 195902, 195903, 195904, 195905, 195906, 195907, 195908, 195909, 195910, 195911, 195912, 195913, 195914, 195915, 195916 were selected from the ORFs having the nucleotide sequence represented by SEQ ID NO: 195600.

The probes having the nucleotide sequence represented by SEQ ID NOS: 195917, 195918, 195919, 195920, 195921, 195922, 195923, 195924, 195925, 195926, 195927, 195928, 195929, 195930, 195931, 195932, 195933, 195934, 195935, 195936 were selected from the ORFs having the nucleotide sequence represented by SEQ ID NO: 195601.

The probes having the nucleotide sequence represented by SEQ ID NOS: 195937, 195938, 195939, 195940, 195941, 195942, 195943, 195944, 195945, 195946, 195947, 195948, 195949, 195950, 195951, 195952, 195953, 195954, 195955, 195956 were selected from the ORFs having the nucleotide sequence represented by SEQ ID NO: 195602.

The probes having the nucleotide sequence represented by SEQ ID NOS: 195957, 195958, 195959, 195960, 195961, 195962, 95963, 195964, 195965, 195966, 195967, 195968, 195969, 195970, 195971, 195972, 195973, 195974, 195975, 195976 were selected from the ORFs having the nucleotide sequence represented by SEQ ID NO: 195603.

The probes having the nucleotide sequence represented by SEQ ID NOS: 195977, 195978, 195979, 195980, 195981, 195982, 195983, 195984, 195985, 195986, 195987, 195988, 195989, 195990, 195991, 195992, 195993, 195994, 195995, 195996 were selected from the ORFs having the nucleotide sequence represented by SEQ ID NO: 195604.

The probes having the nucleotide sequence represented by SEQ ID NOS: 195997, 195998, 195999, 196000, 196001, 196002, 196003, 196004, 196005, 196006, 196007, 196008, 196009, 196010, 196011, 196012, 196013, 196014, 196015, 196016 were selected from the ORFs having the nucleotide sequence represented by SEQ ID NO: 195605.

The probes having the nucleotide sequence represented by SEQ ID NOS: 196017, 196018, 196019, 196020, 196021, 196022, 196023, 196024, 196025, 196026, 196027, 196028, 196029, 196030, 196031, 196032, 196033, 196034, 196035, 196036 were selected from the ORFs having the nucleotide sequence represented by SEQ ID NO: 195606.

The probes having the nucleotide sequence represented by SEQ ID NOS: 196037, 196038, 196039, 196040, 196041, 196042, 196043, 196044, 196045, 196046, 196047, 196048, 196049, 196050, 196051, 196052, 196053, 196054, 196055, 196056 were selected from the ORFs having the nucleotide sequence represented by SEQ ID NO: 195607.

The probes having the nucleotide sequence represented by SEQ ID NOS: 196057, 196058, 196059, 196060, 196061, 196062, 196063, 196064, 196065, 196066, 196067, 196068, 196069, 196070, 196071, 196072, 196073, 196074, 196075, 196076 were selected from the ORFs having the nucleotide sequence represented by SEQ ID NO: 195608.

The probes having the nucleotide sequence represented by SEQ ID NOS: 196077, 196078, 196079, 196080, 196081, 196082, 196083, 196084, 196085, 196086, 196087, 196088, 196089, 196090, 196091, 196092, 196093, 196094, 196095, 196096 were selected from the ORFs having the nucleotide sequences represented by SEQ ID NO: 195609.

The probes having the nucleotide sequence represented by SEQ ID NOS: 196097, 196098, 196099, 196100, 196101, 196102, 196103, 196104, 196105, 196106, 196107, 196108, 196109, 196110, 196111, 196112, 196113, 196114, 196115, 196116 were selected from the ORFs having the nucleotide sequence represented by SEQ ID NO: 195610.

The probes having the nucleotide sequence represented by SEQ ID NOS: 196117, 196118, 196119, 196120, 196121, 196122, 196123, 196124, 196125, 196126, 196127, 196128, 196129, 196130, 196131, 196132, 196133, 196134, 196135, 196136 were selected from the ORFs having the nucleotide sequence represented by SEQ ID NO: 195611.

The probes having the nucleotide sequence represented by SEQ ID NOS: 196137, 196138, 196139, 196140, 196141, 196142, 196143, 196144, 196145, 196146, 196147, 196148, 196149, 196150, 196151, 196152, 196153, 196154, 196155, 196156 were selected from the ORFs having the nucleotide sequence represented by SEQ ID NO: 195612.

The probes having the nucleotide sequence represented by SEQ ID NOS: 196157, 196158, 196159, 196160, 196161, 196162, 196163, 196164, 196165, 196166, 196167, 196168, 196169, 196170, 196171, 196172, 196173, 196174, 196175, 196176 were selected from the ORFs having the nucleotide sequence represented by SEQ ID NO: 195613.

The probes having the nucleotide sequence represented by SEQ ID NOS: 196177, 196178, 196179, 196180, 196181, 196182, 196183, 196184, 196185, 196186, 196187, 196188, 196189, 196190, 196191, 196192, 196193, 196194, 196195, 196196 were selected from the ORFs having the nucleotide sequence represented by SEQ ID NO: 195614.

The probes having the nucleotide sequence represented by SEQ ID NOS: 196197, 196198, 196199, 196200, 196201, 196202, 196203, 196204, 196205, 196206, 196207, 196208, 196209, 196210, 196211, 196212, 196213, 196214, 196215, 196216 were selected from the ORFs having the nucleotide sequence represented by SEQ ID NO: 195615.

The probes having the nucleotide sequence represented by SEQ ID NOS: 196217, 196218, 196219, 196220, 196221, 196222, 196223, 196224, 196225, 196226, 196227, 196228, 196229, 196230, 196231, 196232, 196233, 196234, 196235, 196236 were selected from the ORFs having the nucleotide sequence represented by SEQ ID NO: 195616.

The probes having the nucleotide sequence represented by SEQ ID NOS: 196237, 196238, 196239, 196240, 196241, 196242, 196243, 196244, 196245, 196246, 196247, 196248, 196249, 196250, 196251, 196252, 196253, 196254, 196255, 196256 were selected from the ORFs having the nucleotide sequence represented by SEQ ID NO: 195617.

The probes having the nucleotide sequence represented by SEQ ID NOS: 196257, 196258, 196259, 196260, 196261, 196262, 196263, 196264, 196265, 196266, 196267, 196268, 196269, 196270, 196271, 196272, 196273, 196274, 196275, 196276 were selected from the ORFs having the nucleotide sequence represented by SEQ ID NO: 195618.

The probes having the nucleotide sequence represented by SEQ ID NOS: 196277, 196278, 196279, 196280, 196281, 196282, 196283, 196284, 196285, 196286, 196287, 196288, 196289, 196290, 196291, 196292, 196293, 196294, 196295, 196296 were selected from the ORFs having the nucleotide sequence represented by SEQ ID NO: 195619.

The probes having the nucleotide sequence represented by SEQ ID NOS: 196297, 196298, 196299, 196300, 196301, 196302, 196303, 196304, 196305, 196306, 196307, 196308, 196309, 196310, 196311, 196312, 196313, 196314, 196315, 196316 were selected from the ORFs having the nucleotide sequence represented by SEQ ID NO: 195620.

The probes having the nucleotide sequence represented by SEQ ID NOS: 196317, 196318, 196319, 196320, 196321, 196322, 196323, 196324, 196325, 196326, 196327, 196328, 196329, 196330, 196331, 196332, 196333, 196334, 196335, 196336 were selected from the ORFs having the nucleotide sequence represented by SEQ ID NO: 195621.

The probes having the nucleotide sequence represented by SEQ ID NOS: 196337, 196338, 196339, 196340, 196341, 196342, 196343, 196344, 196345, 196346, 196347, 196348, 196349, 196350, 196351, 196352, 196353,. 196354, 196355, 196356, 196357, 196358, 196359, 196360, 196361, 196362, 196363, 196364, 196365, 196366, 196367, 196368, 196369, 196370, 196371, 196372, 196373, 196374, 196375, 196376, 196377, 196378, 196379, 196380, 196381, 196382, 196383, 196384, 196385, 196386 were selected from the ORFs having the nucleotide sequence represented by SEQ ID NO: 195622.

The probes having the nucleotide sequence represented by SEQ ID NOS: 196387, 196388, 196389, 196390, 196391, 196392, 196393, 196394, 196395, 196396, 196397, 196398, 196399, 196400, 196401, 196402, 196403, 196404, 196405, 196406, 196407, 196408, 196409, 196410, 196411, 196412, 196413, 196414, 196415, 196416, 196417, 196418, 196419, 196420, 196421, 196422, 196423, 196424, 196425, 196426, 196427, 196428, 196429, 196430, 196431, 196432, 196433, 196434, 196435, 196436, 196437 were selected from the ORFs having the nucleotide sequence represented by SEQ ID NO: 195623.

The probes having the nucleotide sequence represented by SEQ ID NOS: 196438, 196439, 196440, 196441, 196442, 196443, 25 196444, 196445, 196446, 196447, 196448, 196449, 196450, 196451, 196452, 196453, 196454, 196455, 196456, 196457, 196458, 196459, 196460, 196461, 196462, 196463, 196464, 196465, 196466, 196467, 196468, 196469, 196470, 196471, 96472, 196473, 196474, 196475, 196476, 196477, 196478, 196479, 196480, 196481, 196482, 196483, 196484, 196485, 196486, 196487 were selected from the ORFs having the nucleotide sequence represented by SEQ ID NO: 195624.

The probes having the nucleotide sequence represented by SEQ ID NOS: 196488, 196489, 196490, 196491, 196492, 196493, 196494, 196495, 196496, 196497, 196498, 196499, 196500, 196501, 196502, 196503, 196504,. 196505, 196506, 196507, 196508, 196509, 196510, 196511, 196512, 196513, 196514, 196515, 196516, 196517, 196518, 196519, 196520, 196521, 196522, 196523, 196524, 196525, 196526, 196527, 196528, 196529, 196530, 196531, 196532, 196533, 196534, 196535, 196536, 196537 were selected from the ORFs having the nucleotide sequence represented by SEQ ID NO: 195625.

The probes having the nucleotide sequence represented by SEQ ID NOS: 196538, 196539, 196540, 196541, 196542, 196543, 196544, 196545, 196546, 196547, 196548, 196549, 196550, 196551, 196552, 196553, 196554, 196555, 196556, 196557, 196558, 196559, 196560, 196561, 196562, 196563, 196564, 196565, 196566, 196567, 196568, 196569, 196570, 196571, 196572, 196573, 196574, 196575, 196576, 196577, 196578, 196579, 196580, 196581, 196582, 196583, 196584, 196585, 196586, 196587, 196588 were selected from the ORFs having the nucleotide sequence represented by SEQ ID NO: 195626.

The probes having the nucleotide sequence represented by SEQ ID NOS: 196589, 196590, 196591, 196592, 196593, 196594, 196595, 196596, 196597, 196598, 196599, 196600, 196601, 196602, 196603, 196604, 196605, 196606, 196607, 196608, 196609, 196610, 196611, 196612, 196613, 196614, 196615, 196616, 196617, 196618, 196619, 196620, 196621, 196622, 196623, 196624, 196625, 196626, 196627, 196628, 196629, 196630, 196631, 196632, 196633, 196634, 196635, 196636, 196637, 196638 were selected from the ORFs having the nucleotide sequence represented by SEQ ID NO: 195627.

The probes having the nucleotide sequence represented by SEQ ID NOS: 196639, 196640, 196641, 196642, 196643, 196644, 196645, 196646, 196647, 196648, 196649, 196650, 196651, 196652, 196653, 196654, 196655, 196656, 196657, 196658, 196659, 196660, 196661, 196662, 196663, 196664, 196665, 196666, 196667, 196668, 196669, 196670, 196671, 196672, 196673, 196674, 196675, 196676, 196677, 196678, 196679, 196680, 196681, 196682, 196683, 196684, 196685, 196686, 196687, 196688, 196689 were selected from the ORFs having the nucleotide sequence represented by SEQ ID NO: 195628.

The probes having the nucleotide sequence represented by SEQ ID NOS: 196690, 196691, 196692, 196693, 196694, 196695, 196696, 196697, 196698, 196699, 196700, 196701, 196702, 196703, 196704, 196705, 196706, 196707, 196708, 196709, 196710, 196711, 196712, 196713, 196714, 196715, 196716, 196717, 196718, 196719, 196720, 196721, 196722, 196723, 196724, 196725, 196726, 196727, 196728, 196729, 196730, 196731, 196732, 196733, 196734, 196735, 196736, 196737, 196738, 196739, 196740 were selected from the ORFs having the nucleotide sequence represented by SEQ ID NO: 195629.

The probes having the nucleotide sequence represented by SEQ ID NOS: 196741, 196742, 196743, 196744, 196745, 196746, 196747, 196748, 196749, 196750, 196751, 196752, 196753, 196754, 196755, 196756, 196757, 196758, 196759, 196760, 196761, 196762, 196763, 196764, 196765, 196766, 196767, 196768, 196769, 196770, 196771, 196772, 196773, 196774, 196775, 196776, 196777, 196778, 196779, 196780, 196781, 196782, 196783, 196784, 196785, 196786, 196787, 196788, 196789, 196790 were selected from the ORFs having the nucleotide sequence represented by SEQ ID NO: 195630.

The probes having the nucleotide sequence represented by SEQ ID NOS: 196791, 196792, 196793, 196794, 196795, 196796, 196797, 196798, 196799, 196800, 196801 were selected from the ORFs having the nucleotide sequence represented by SEQ ID NO: 195631.

The probes having the nucleotide sequence represented by SEQ ID NOS: 196802, 196803, 196804, 196805, 196806, 196807, 196808, 196809, 196810, 196811, 196812 were selected from the ORFs having the nucleotide sequence represented by SEQ ID NO: 195632.

The probes having the nucleotide sequence represented by SEQ ID NOS: 196813, 196814, 196815, 196816, 196817, 196818, 196819, 196820, 196821, 196822, 196823 were selected from the ORFs having the nucleotide sequence represented by SEQ ID NO: 195633.

The probes having the nucleotide sequence represented by SEQ ID NOS: 196824, 196825, 196826, 196827, 196828, 196829, 196830, 196831, 196832, 196833, 196834 were selected from the ORFs having the nucleotide sequence represented by SEQ ID NO: 195634.

The probes having the nucleotide sequence represented by SEQ ID NOS: 196835, 196836, 196837, 196838, 196839, 196840, 196841, 196842, 196843, 196844, 196845 were selected from the ORFs having the nucleotide sequence represented by SEQ ID NO: 195635.

The probes having the nucleotide sequence represented by SEQ ID NOS: 196846, 196847, 196848, 196849, 196850, 196851, 196852, 196853, 196854, 196855, 196856 were selected from the ORFs having the nucleotide sequence represented by SEQ ID NO: 195636.

The probes having the nucleotide sequence represented by SEQ ID NOS: 196857, 196858, 196859, 196860, 196861, 196862, 196863, 196864, 196865, 196866, 196867 were selected from the ORFs having the nucleotide sequence represented by SEQ ID NO: 195637.

The probes having the nucleotide sequence represented by SEQ ID NOS: 196868, 196869, 196870, 196871, 196872, 196873, 196874, 196875, 196876, 196877, 196878 were selected from the ORFs having the nucleotide sequence represented by SEQ ID NO: 195638.

The probes having the nucleotide sequence represented by SEQ ID NOS: 196879, 196880, 196881, 196882, 196883, 196884, 196885, 196886, 196887, 196888, 196889 were selected from the ORFs having the nucleotide sequence represented by SEQ ID NO: 195639.

The probes having the nucleotide sequence represented by SEQ ID NOS: 196890, 196891, 196892, 196893, 196894, 196895, 196896, 196897, 196898, 196899, 196900 were selected from the ORFs having the nucleotide sequence represented by SEQ ID NO: 195640.

The probes having the nucleotide sequence represented by SEQ ID NOS: 196901, 196902, 196903, 196904, 196905, 196906, 196907, 196908, 196909, 196910, 196911 were selected from the ORFs having the nucleotide sequence represented by SEQ ID NO: 195641.

The probes having the nucleotide sequence represented by SEQ ID NOS: 196912, 196913, 196914, 196915, 196916, 196917, 196918, 196919, 196920, 196921, 196922 were selected from the ORFs having the nucleotide sequence represented by SEQ ID NO: 195642.

The probes having the nucleotide sequence represented by SEQ ID NOS: 196923, 196924, 196925, 196926, 196927, 196928, 196929, 196930, 196931, 196932, 196933 were selected from the ORFs having the nucleotide sequence represented by SEQ ID NO: 195643.

The probes having the nucleotide sequence represented by SEQ ID NOS: 196934, 196935, 196936, 196937, 196938, 196939, 196940, 196941, 196942, 196943, 196944 were selected from the ORFs having the nucleotide sequence represented by SEQ ID NO: 195644.

The probes having the nucleotide sequence represented by SEQ ID NOS: 196945, 196946, 196947, 196948, 196949, 196950, 196951, 196952, 196953, 196954, 196955 were selected from the ORFs having the nucleotide sequence represented by SEQ ID NO: 195645.

The probes having the nucleotide sequence represented by SEQ ID NOS: 196956, 196957, 196958, 196959, 196960, 196961, 196962, 196963, 196964, 196965, 196966 were selected from the ORFs having the nucleotide sequence represented by SEQ ID NO: 195646.

The probes having the nucleotide sequence represented by SEQ ID NOS: 196967, 196968, 196969, 196970, 196971, 196972, 196973, 196974, 196975, 196976, 196977 were selected from the ORFs having the nucleotide sequence represented by SEQ ID NO: 195647.

The probes having the nucleotide sequence represented by SEQ ID NOS: 196978, 196979, 196980, 196981, 196982, 196983, 196984, 196985, 196986, 196987, 196988 were selected from the ORFs having the nucleotide sequence represented by SEQ ID NO: 195648.

The probes having the nucleotide sequence represented by SEQ ID NOS: 196989, 196990, 196991, 196992, 196993, 196994, 196995, 196996, 196997, 196998, 196999 were selected from the ORFs having the nucleotide sequence represented by SEQ ID NO: 195649.

The probes having the nucleotide sequence represented by SEQ ID NOS: 197000, 197001, 197002, 197003, 197004, 197005, 197006, 197007, 197008, 197009, 197010 were selected from the ORFs having the nucleotide sequence represented by SEQ ID NO: 195650.

The probes having the nucleotide sequence represented by SEQ ID NOS: 197011, 197012, 197013, 197014, 197015, 197016, 197017, 197018, 197019, 197020, 197021 were selected from the ORFs having the nucleotide sequence represented by SEQ ID NO: 195651.

### Example 18

### Identification Molecular Markers that are Highly Inducible in Zinc Depleted Condition

1. Preparation of mRNA

Strain 34/70 was grown overnight in LZMM medium+40 μM zinc sulfate at 30° C. with shaking. LZMM medium contains 0.17% yeast nitrogen base w/o amino acids (manufactured by DIFCO), 0.5% ammonium sulfate, 20 mM sodium citrate (pH 4.2), 125 μM MnCl2, 10 μM FeCl2, 2% maltose, 10 mM EDTA (pH 8.0). Cells were harvested and washed three times with sterile distilled water before inoculation to an optical density (OD600) of 0.25 in 500 ml of 1) zinc depleted medium (LZMM medium), 2) zinc replete medium (LZMM+40 μM zinc sulfate), 3) oxidative stress medium (LZMM+40 μM zinc sulfate+2 mM H₂O₂), 4) carbon starvation medium (deleting maltose from above LZMM+40 μM zinc sulfate). Cells were grown at 30° C. for 6 hours and harvested for RNA preparation.

Preparation of total RNA was carried out using RNeasy® Mini Kit (manufactured by QIAGEN) according to a manual. Preparation of Poly(A)+mRNA from total RNA was carried out using Oligotex Direct mRNA kit (manufactured by QIAGEN) according to a manual.

2. Synthesis of Biotin-Labeled cRNA

Synthesis of Biotin-Labeled cRNA was carried out using BioArray HighYield RNA Transcript Labeling Kit (manufactured by Affymetrix) according to a manual.

3. Hybridization

5 μg of Biotin-Labeled cRNA, 1.7 μl of 3 nM Control Oligonucleotide B2 (manufactured by Affymetrix), 5 μl of 20× Eukaryotic Hybridization Controls (manufactured by Affymetrix), 1 μl of 10 mg/ml Herring Sperm DNA (manufactured by Affymetrix), 1 μl of 50 mg/ml Acetylated BSA (manufactured by Affymetrix), 50 μl of 2× Hybridization buffer (manufactured by Affymetrix), and water (manufactured by Affymetrix) to give final volume of 100 μl were mixed and hybridized to the DNA array according to a Technical Manual of Affymetrix.

After 16 hours of hybridization, hybridization cocktail was removed and the DNA array was washed using the GeneChip® Fludics Station (manufactured by Affymetrix), and stained with 600 μl of Streptavidin Phycoerythrin (300 μl of 2× MES Stain Buffer (manufactured by Affymetrix), 24 μl of 50 mg/ml acetylated BSA (manufactured by Affymetrix), 6 μl of 1 mg/ml StreptAvidin-Phycoerythrin (manufactured by Affymetrix), 270 μl of water (manufactured by Affymetrix) according to a Technical Manual of Affymetrix.

4. Data analysis

Detection of the signal intensity of the DNA array was carried out using Gene Chip Analysis Basic System and analysis software (GCOS; GeneChip Operating Software 1.0) according to a Technical Manual of Affymetrix.

Normalization was carried out using the All Probe Sets method in GCOS to adjust a signal in comparison analysis. The comparison files of gene expression which were compared (1) zinc depleted condition to zinc replete condition, (2) oxidative stress condition to zinc replete condition and (3) carbon starvation condition to zinc replete condition were created using GCOS, respectively. The genes whose expressions were increased by more than 0.3 at signal log ratio only in above comparison (1) were shown in Table 4.

Sc-1159-1_at, Sc-1161-1_at, Sc-5030-1_at, Sc-2123-1_at is Sc YGL258W, Sc YGL256W, Sc YOL154W, Sc YKL175W, respectively. And it is known that these genes are transcriptionally induced in zinc depleted condition (Higgins, V. J. et al., Appl. Environ. Microbiol., 69: 7535-7540(2003)). Lg-4216-1_s_at was designed to correspond to Non-Sc YKL175W whose function was assigned zinc ion transporter activity. It is known that zinc ion transporter is transcriptionally induced in zinc depleted condition.

In conclusion, it is shown that the molecular markers that are highly induced in zinc depleted condition can be identified using the bottom fermenting yeast DNA array.

TABLE 4

| | (1) zinc deplete/zinc replete | | | (2) oxidative stress/zinc replete | | | | | | | |
|---|---|---|---|---|---|---|---|---|---|---|---|
| | Signal | | | Signal | | | (3) carbon starvation/zinc replete | | | annotation | |
| probe set | Log Ratio | Detection | Change | Log Ratio | Detection | Change | Signal Log Ratio | Detection | Change | Gene name | Type |
| Sc-1159-1_at | 3.1 | P | I | −0.6 | A | NC | −0.5 | A | NC | YGL258W | Sc |
| Lg-4570-1_at | 1.2 | P | I | 0.1 | P | NC | −0.7 | A | D | YNL254C | Non-Sc |
| Sc-1161-1_at | 1.1 | P | I | −1.1 | P | D | −1.2 | P | D | YGL256W | Sc |
| Lg-3847-1_at | 0.9 | P | I | −0.6 | P | D | −1.1 | P | D | YGL256W | Non-Sc |
| Sc-2889-1_at | 0.7 | P | I | −0.5 | P | D | −1.8 | P | D | YNL254C | Sc |
| Lg-4216-1_s_at | 0.6 | P | I | −0.6 | P | D | −0.4 | P | D | YKL175W | Non-Sc |
| Sc-5030-1_at | 0.5 | P | I | −3.6 | P | D | −3.8 | P | D | YOL154W | Sc |
| Sc-1160-1_at | 0.4 | P | I | −1.1 | P | D | −0.9 | P | D | YGL257C | Sc |
| Lg-1751-1_at | 0.4 | P | I | −1 | P | D | −0.7 | P | D | YLR209C | Non-Sc |
| Sc-3567-1_at | 0.4 | P | I | 0.2 | P | NC | −0.4 | P | NC | YPL148C | Sc |
| Lg-3161-1_at | 0.4 | P | I | −0.8 | P | D | −0.9 | P | D | YMR020W | Non-Sc |
| Sc-3984-1_x_at | 0.4 | P | I | 0.2 | P | NC | −1.3 | P | D | YDL150W | Sc |
| Lg-4390-2_x_at | 0.4 | P | I | 0.3 | P | NC | −0.5 | P | NC | YLR339C | Non-Sc |
| Sc-4798-1_at | 0.4 | P | I | 0.2 | P | NC | −2.3 | P | D | YLR435W | Sc |
| Lg-5145-1_s_at | 0.4 | P | I | 0.2 | P | NC | −2.3 | P | D | YDR312W | Non-Sc |
| Lg-139-1_at | 0.3 | P | I | −0.3 | P | NC | −4.1 | A | D | YBR104W | Non-Sc |
| Lg-467-1_at | 0.3 | P | I | 0.1 | P | NC | −2 | P | D | YDR161W | Non-Sc |
| Sc-1412-1_at | 0.3 | P | I | 0 | P | NC | −2 | P | D | YGR081C | Sc |
| Lg-961-1_at | 0.3 | P | I | 0.2 | P | NC | −1.5 | P | D | YGR103W | Non-Sc |
| Sc-2122-1_at | 0.3 | P | I | −0.3 | P | NC | 0.1 | P | NC | YKL176C | Sc |
| Sc-2123-1_at | 0.3 | P | I | −0.5 | P | D | −1.1 | P | D | YKL175W | Sc |
| Sc-2209-1_at | 0.3 | P | I | 0.2 | P | NC | −1.7 | P | D | YKL072W | Sc |
| Sc-2356-1_at | 0.3 | P | I | −0.1 | P | NC | −2.4 | P | D | YLR129W | Sc |
| Lg-1955-1_at | 0.3 | P | I | −0.1 | P | NC | 0 | P | NC | YMR096W | Non-Sc |
| Sc-2890-1_at | 0.3 | P | I | −0.3 | P | NC | −1.2 | P | D | YNL253W | Sc |
| Lg-2100-1_at | 0.3 | P | I | −0.8 | P | D | −1.7 | P | D | YNL217W | Non-Sc |
| Sc-2920-1_at | 0.3 | P | I | 0.3 | P | MI | −2.4 | P | D | YNL207W | Sc |
| Lg-2258-1_at | 0.3 | P | I | 0.1 | P | NC | −1.8 | P | D | YOL125W | Non-Sc |
| Sc-3203-1_at | 0.3 | P | I | 0.1 | P | NC | −0.8 | P | D | YOL022C | Sc |
| Sc-3651-1_s_at | 0.3 | P | I | 0.1 | P | NC | −0.1 | P | NC | YPR044C | Sc |
| Lg-2648-1_at | 0.3 | P | I | 0.1 | P | NC | −2.1 | P | D | YPR048W | Non-Sc |
| Lg-3014-1_at | 0.3 | P | I | −0.7 | P | D | −0.3 | P | NC | YJL055W | Non-Sc |
| Sc-4034-1_at | 0.3 | P | I | 0.1 | P | NC | 0.3 | P | NC | YDR017C | Sc |
| Lg-3620-1_at | 0.3 | P | I | 0.4 | P | NC | −2.1 | P | D | YDR087C | Non-Sc |
| Sc-4163-1_at | 0.3 | P | I | 0.1 | P | NC | −2.5 | M | D | YDR449C | Sc |
| Sc-4365-1_at | 0.3 | P | I | 0.3 | P | NC | −1.4 | P | D | YGR145W | Sc |
| Sc-4454-1_at | 0.3 | P | I | 0.4 | P | NC | −1.8 | P | D | YHR197W | Sc |
| Lg-4608-2_at | 0.3 | P | I | −0.3 | P | D | −1.9 | P | D | YNL112W | Non-Sc |
| Lg-4622-1_at | 0.3 | P | I | 0.1 | P | NC | −2.5 | P | D | YNL062C | Non-Sc |
| Sc-5321-1_at | 0.3 | P | I | 0.4 | P | NC | −1.1 | P | D | YGR272C | Sc |
| Lg-5125-1_at | 0.3 | P | I | 0.2 | P | NC | −2.4 | P | D | YDR101C | Non-Sc |

Signal Log Ratio (2n) indicates the magnitude and direction of a transcript when two arrays were compared. Detection indicates whether a transcript was reliably detected (P; Present) or not detected (A; Absent) based on the Detection p-value calculated by Detection Algorithm with default paramater in GCOS. Change indicates whether a transcript was reliably increased (I; Increase) or decreased (D; Decrease) or not changed (NC; No Change) based on the Change p-value calculated by Change Algorithm with default paramater in GCOS. Gene name indicates where the corresponding probe set was designed. Type indicates whether a gene is Sc ORF (Sc) or Non-Sc ORF (Non-Sc).

### Example 19

### Gene Expression Analysis of Brewing Yeast Under Beer Fermenting Condition

Fermentation test using strain 34/70 was carried out under the following condition.

Original extract: 12.84%

Fermentation scale: 2 liters

Dissolved oxygen concentration: about 9 ppm

Fermentation temperature: 15° C.

Pitching rate: 10 g of wet yeast cells/2 L of wort

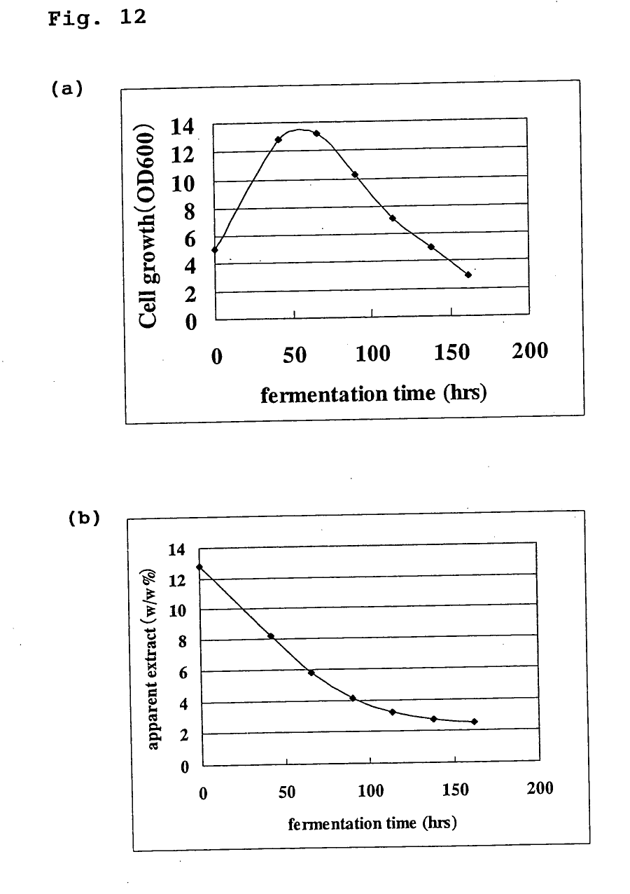

Wort is periodically sampled and monitored in cell growth (OD 600) (FIG. 12-(a)) and apparent extract (FIG. 12-b)). mRNA was extracted from the cells withdrawn from the fermentation tubes 42 hours after inoculation, biotin labeled and hybridized to the bottom fermenting yeast DNA array as described in example 18. Detection of the signal intensity was carried out using a Gene Chip Analysis Basic System and analysis soft ware (GCOS; GeneChip Operating Software 1.0) manufactured by Affymetrix.

There were more than a few genes whose Sc type probe sets and non-Sc type probe sets showed quite different signal intensities. Examples of SSU1 genes and MET14 genes, which are related to sulfite production during beer fermentation are shown in Table 5. In the case of SSU1 genes and MET14 genes of strain 34/70, the expressions of non-Sc type were higher than those of Sc type, by 3.4-fold and 7-fold, respectively.

In order to confirm this difference is due to neither the difference of hybridization efficiency of each probe set nor cross hybridization between Sc and non-Sc type probe sets, comparative genomic hybridization with the bottom fermenting yeast DNA array was carried out using strain 34/70, a laboratory strain (S. cerevisiae) S288C and S. carlsbergensis strain IF011023. The preparation of genomic DNA, hybridization to DNA array and detection of the signal intensities were carried out with the method described before. As shown in Table 6, the ratio of signal intensity of non-Sc type to that of Sc type was 1.0 for SSU1 genes and 1.3 for MET14 genes in strain 34/70. This result shows that hybridization efficiencies of Sc and non-Sc probe sets were almost same.

Furthermore, strain S288C, which does not have non-Sc type genes, showed very low signal intensities to non-Sc type probe sets, and strain IF011023, which does not have Sc type SSU1 gene and Sc type MET14, showed very low signal intensities to Sc type SSU1 and Sc type MET14 probe sets. These results clearly show that cross hybridization did not occur between Sc and non-Sc type probe sets.

From these results, in strain 34/70, the expressions of non-Sc SSU1 and non-Sc MET14 were significantly higher than those of Sc SSU1 and Sc MET14, respectively. These genes are thought to be candidates which contribute to the high sulfite production ability of bottom fermenting yeast.

In conclusion, it was revealed that gene expression analysis of brewing yeast strains using the bottom fermenting yeast DNA array was useful for the selection of gene(s) for functional analysis.

TABLE 5

| | gene | | | |
|---|---|---|---|---|
| | Sc_SSU1 | non_Sc_SSU1 | Sc_MET14 | non_Sc_MET14 |
| | probe set | | | |
| | Sc-3594-1 at | Lg-3333-1 at | Sc-2246-1 at | Lg-1564-1 at |
| signal | 145.2 | 490.4 | 177.3 | 1245.8 |
| inten- | | | | |
| sity | | | | |

TABLE 6

| | gene | Sc_SSUI | non_Sc_SSUI | Sc_MET14 | non_Sc_MET14 |
|---|---|---|---|---|---|
| strains | probe set | Sc-3594-1_at | Lg-3333-1_at | Sc-2246-1_at | Lg-1564-1_at |
| 34/70 | signal | 360.9 | 356.8 | 244.2 | 324.8 |
| S288C | intensity | 516.2 | 6.5 | 405.3 | 13.4 |
| S. carlsbergensis IFO11023 | | 8.5 | 746.9 | 6.8 | 508.4 |

### Example 20

### Classification of Brewing Strains by Comparative Genomic Hybridization with the Bottom Fermenting Yeast DNA Array

Preparation of yeast genomic DNA and hybridization to the DNA array was carried out as described in (Example 8). Detection of the signal intensity of the DNA array was carried out using a Gene Chip Analysis Basic System and analysis soft ware (GCOS; GeneChip Operating Software 1.0) manufactured by Affymetrix. The percentage of probes, which the DNA of brewing yeast hybridized was calculated and the identity between strain 34/70 and the tested strain was estimated as shown in Table 7. Strains BH225, BH232 and BH235 hybridized to more than 99% of both Sc type and non-Sc type probes of the bottom fermenting yeast DNA array. It suggests that these strains are very close to strain 34/70, and that this array is useful for the gene expression analysis of these strains. On the other hand, strain BH212 showed relatively low (97.8 and 97.7% for Sc type and non-Sc type probe, respectively) percentage of hybridization, which means this strain is a little bit different from strain 34/70. From these results, relationship among lager brewing strains can be estimated and classification of lager brewing strains can be carried out.

From the result of analysis of strain BH212, some loci which showed very low signal intensities were found. They may be lost in strain BH212 or their sequences may be different from those of strain 34/70. In contrast, some loci which showed high signal intensities were also found. They may be high in copy number in strain BH212. Such loci can be selected for functional analysis because they may contribute to the difference of fermentation character between strain BH212 and strain 34/70.

TABLE 7
percentage of hybridized probes

| | Strain No. | | | | |
|---|---|---|---|---|---|
| | 34/70 | BH225 | BH232 | BH235 | BH212 |
| Sc type | 99.6 | 99.8 | 99.8 | 99.8 | 97.8 |
| Non-Sc | 99.5 | 99.9 | 99.9 | 99.6 | 97.7 |
| type | | | | | |

### Example 21

### Detection of Nucleotide Polymorphism

Furthermore, (single) nucleotide polymorphism was detectable by the analysis of comparative genomic hybridization. The sets of oligonucleotides for each probe consist of Perfect Match oligonucleotide (PM) which is identical to the sequence of strain 34/70 and MisMatch oligonucleotide (MM) which contains a single base mismatch in the central position of the oligonucleotide. Genomic DNA of a laboratory strain S288C was hybridized to the bottom fermenting yeast DNA array. As shown in Table 8, probes which showed higher (more than 5-fold) signal in MM than in PM had single nucleotide polymorphism.

TABLE 8

| | signal of | signal of MM | | |
|---|---|---|---|---|
| probe | PM probe | probe | | |
| Mt6s at 653 337 | 11238 | 63439 | DNA sequence of PM | GAATCAATTAACTTATGGTTTCTTA |
| | | | | \|\|\|\|\|\|\|\|\|\|\|\| \|\|\|\|\|\|\|\|\|\|\|\|\| |
| | | | DNA sequence of tested strain | GAATCAATTAACATATGGTTTCTTA |
| Sc-21 33-1 _at 59 _283 | 4725 | 27825 | DNA sequence of PM | CCCCTTAGTGGCGAAGATATCTTTT |
| | | | | \| \|\|\| \|\|\| \| \| \|\|\|\| \|\|\|\| |
| | | | DNA sequence of tested strain | GCTCTTGGTGACCAGGATACTTTTT |

### INDUSTRIAL APPLICABILITY

A database compiling the data of the whole genome sequences of an industrial yeast or, particularly, of a brewing yeast used for the production of alcoholic beverages such as beer is prepared. Using such a database, genes of brewing yeast are selected, and functions of the genes are analyzed by disruption or overexpression in yeast cells, and find genes participating in the desired brewing character. Furthermore, it is possible to breed yeast strains by controlling the expression of the said genes, and produce an alcohol or an alcoholic beverage where productivity and quality are improved, such as alcoholic beverages with high concentration of sulfite which shows antioxidant activity in the product, excellent flavor stability and a longer quality preservation period.

Based on the database compiling the data of the whole genome sequences of an industrial yeast or, particularly, of a brewing yeast, a DNA array is produced. Using the DNA array, it is possible to analyze functions of the genes, classify industrial yeasts and detect nucleotide polymorphism and so on.

1. A method for analyzing gene of an industrial yeast comprising
(a) determining nucleotide sequence of the whole genome sequence of the industrial yeast; and
(c-1) selecting a gene of the industrial yeast encoding an amino acid sequence having 70 to 97% identity to an amino acid sequence encoded by the gene of Saccharomyces cerevisiae, or
(c-2) selecting a gene of the industrial yeast consisting of a nucleotide sequence having 60 to 94% identity to the nucleotide sequence of the gene of Saccharomyces cerevisiae.

2. The analyzing method according to claim 1, comprising,
(b) comparing the genome sequence with the whole genome sequence of Saccharomyces cerevisiae after the step (a) and before the step (c-1) or (c-2).

3. The analyzing method according to claim 1, comprising
(d) carrying out functional analysis of the selected gene
after the step (c-1) or (c-2).

4. The analyzing method according to claim 3, which comprises screening genes participating in increase in productivity and/or improvement in flavor in the production of an alcohol or an alcoholic beverage by the functional analysis of the step (d).

5. The analyzing method according to claim 3, wherein functional analysis is performed by disruption of a gene or overexpression of a gene.

6. The analyzing method according to claim 1, wherein the industrial yeast is a brewing yeast.

7. The analyzing method according to claim 6, wherein the brewing yeast is a beer yeast.

8. The analyzing method according to claim 7, wherein the beer yeast is a bottom fermenting yeast.

9. A gene of the industrial yeast encoding an amino acid sequence having 70 to 97% identity to an amino acid sequence encoded by the gene of Saccharomyces cerevisiae, or consisting of a nucleotide sequence having 60 to 94% identity to the nucleotide sequence of the gene of Saccharomyces cerevisiae, which is obtained by the analyzing method according to any one of claims 1-8.

10. A gene library comprising one or more of genes of the industrial yeast each of which encodes an amino acid sequence having 70 to 97% identity to an amino acid sequence encoded by the gene of Saccharomyces cerevisiae, or consists of a nucleotide sequence having 60 to 94% identity to the nucleotide sequence of the gene of Saccharomyces cerevisiae, which is obtained by the analyzing method according to any one of claim 1.

11. The gene library according to claim 10, wherein the gene of the industrial yeast is comprised in, consists of or has any nucleotide sequence selected from SEQ ID NOs:33 to 6236, SEQ ID NOs:166154 to 166181, SEQ ID NOs:166490 to 167042 and SEQ ID NOs:173125 to 174603.

12. A DNA array comprising one or more of DNAs, wherein each DNA is selected from at least one group of (1) to (4):
(1) DNA consisting of a nucleotide sequence of an open reading frame of the genome sequence of an industrial yeast which encodes an amino acid sequence having 70 to 97% identity to an amino acid sequence encoded by the gene of Saccharomyces cerevisiae, or a nucleotide sequence complementary to the above nucleotide sequence, or a nucleotide sequence of continuous 10 or more nucleotides selected from the above nucleotide sequences;
(2) DNA consisting of a nucleotide sequence of the genome sequence of an industrial yeast other than from open reading frames which consists of a nucleotide sequence having 60 to 94% identity to the nucleotide sequence of the gene of Saccharomyces cerevisiae, or a nucleotide sequence complementary to the above nucleotide sequence, or a nucleotide sequence of continuous 10 or more nucleotides selected from the above nucleotide sequences;
(3) DNA having a nucleotide sequence of any of SEQ ID NOs:166490 to 167042, or DNA having a nucleotide sequence of any of SEQ ID NOs:167043 to 173124; and
(4) DNA having a nucleotide sequence of any of SEQ ID NOs:173125 to 174603, or DNA having a nucleotide sequence of any of SEQ ID NOs:174604 to 190810.

13. The DNA array according to claim 12 comprising DNAs wherein at least one DNA is selected from every group of (1) to (4).

14. The DNA array according to claim 12, wherein the DNAs of (1) is comprised in, consists of or has any nucleotide sequence selected from SEQ ID NOs:33 to 6236 and SEQ ID NOs:166154 to 166181, and SEQ ID NOs:6237 to 75336 and SEQ ID NOs:166182 to 166489.

15. The DNA array according to any one of claim 12, which further comprises, in addition to at least one group of DNAs selected from (1) to (4), DNAs of the following (5) and/or (6):
(5) DNA consisting of a nucleotide sequence of an open reading frame of the genome sequence of an industrial yeast which encodes an amino acid sequence having identity of more than 97% to an amino acid sequence encoded by the gene of Saccharomyces cerevisiae, or a nucleotide sequence complementary to the above nucleotide sequence, or a nucleotide sequence of continuous 10 or more nucleotides selected from the above nucleotide sequences; and
(6) DNA consisting of a nucleotide sequence of the genome sequence of an industrial yeast other than from open reading frames which consists of a nucleotide sequence having identity of more than 94% to the nucleotide sequence of the gene of Saccharomyces cerevisiae, or a nucleotide sequence complementary to the above nucleotide sequence, or a nucleotide sequence of continuous 10 or more nucleotides selected from the above nucleotide sequences, and optionally DNAs of the following (5′) and/or (6′):
(5′) DNA consisting of a nucleotide sequence which contains mismatch of 1 or more base(s) to the DNA of (5), or a nucleotide sequence complementary to the above nucleotide sequence; and
(6′) DNA consisting of a nucleotide sequence which contains mismatch of 1 or more base(s) to the DNA of (6), or a nucleotide sequence complementary to the above nucleotide sequence.

16. The DNA array according to claim 15, wherein the DNAs of (5) has a nucleotide sequence of any one of SEQ ID NOs:75337 to 82784, or SEQ ID NOs:82785 to 166153.

17. The DNA array according to any one of claim 12, which comprises, in addition to at least one group of DNAs selected from (1) to (4), DNAs selected from at least one group of (7) to (10):
(7) DNA consisting of a nucleotide sequence which contains mismatch of 1 or more base(s) to the DNA of (1), or a nucleotide sequence complementary to the above nucleotide sequence;
(8) DNA consisting of a nucleotide sequence which contains mismatch of 1 or more base(s) to the DNA of (2), or a nucleotide sequence complementary to the above nucleotide sequence;
(9) DNA consisting of a nucleotide sequence which contains mismatch of 1 or more base(s) to the DNA of (3), or a nucleotide sequence complementary to the above nucleotide sequence; and
(10) DNA consisting of a nucleotide sequence which contains mismatch of 1 or more base(s) to the DNA of (4), or a nucleotide sequence complementary to the above nucleotide sequence.

18. The DNA array according to claim 17, which comprises, in addition to at least one group of DNAs elected from (1) to (4), DNAs selected from at least one group of (7-1) to (10-1):
(7-1) DNA consisting of a nucleotide sequence which contains mismatch of 1 base to the DNA of (1), or a nucleotide sequence complementary to the above nucleotide sequence;
(8-1) DNA consisting of a nucleotide sequence which contains mismatch of 1 base to the DNA of (2), or a nucleotide sequence complementary to the above nucleotide sequence;
(9-1) DNA consisting of a nucleotide sequence which contains mismatch of 1 base to the DNA of (3), or a nucleotide sequence complementary to the above nucleotide sequence; and
(10-1) DNA consisting of a nucleotide sequence which contains mismatch of 1 base to the DNA of (4), or a nucleotide sequence complementary to the above nucleotide sequence.

19. A DNA array comprising:
I) at least one group of DNAs selected from at least one group of (1) to (4):
(1) DNA consisting of a nucleotide sequence of an open reading frame of the genome sequence of an industrial yeast which encodes an amino acid sequence having 70 to 97% identity to an amino acid sequence encoded by the gene of Saccharomyces cerevisiae, or a nucleotide sequence complementary to the above nucleotide sequence, or a nucleotide sequence of continuous 10 or more nucleotides selected from the above nucleotide sequences;
(2) DNA consisting of a nucleotide sequence of the genome sequence of an industrial yeast other than from open reading frames which consists of a nucleotide sequence having 60 to 94% identity to the nucleotide sequence of the gene of Saccharomyces cerevisiae, or a nucleotide sequence complementary to the above nucleotide sequence, or a nucleotide sequence of continuous 10 or more nucleotides selected from the above nucleotide sequences;
(3) DNA having a nucleotide sequence of any of SEQ ID NOs:166490 to 167042, or DNA having a nucleotide sequence of any of SEQ ID NOs:167043 to 173124; and
(4) DNA having a nucleotide sequence of any of SEQ ID NOs:173125 to 174603, or DNA having a nucleotide sequence of any of SEQ ID NOs:174604 to 190810:
II) at least one group of DNAs selected from at least one group of (7) to (10):
(7) DNA consisting of a nucleotide sequence which contains mismatch of 1 or more base(s) to the DNA of (1), or a nucleotide sequence complementary to the above nucleotide sequence;
(8) DNA consisting of a nucleotide sequence which contains mismatch of 1 or more base(s) to the DNA of (2), or a nucleotide sequence complementary to the above nucleotide sequence;
(9) DNA consisting of a nucleotide sequence which contains mismatch of 1 or more base(s) to the DNA of (3), or a nucleotide sequence complementary to the above nucleotide sequence; and
(10) DNA consisting of a nucleotide sequence which contains mismatch of 1 or more base(s) to the DNA of (4), or a nucleotide sequence complementary to the above nucleotide sequence:
III) DNAs of the following (5) and/or (6):
(5) DNA consisting of a nucleotide sequence of an open reading frame of the genome sequence of an industrial yeast which encodes an amino said sequence having identity of more than 97% to an amino acid sequence encoded by the gene of Saccharomyces cerevisiae, or a nucleotide sequence complementary to the above nucleotide sequence, or a nucleotide sequence of continuous 10 or more nucleotide selected from the above nucleotide sequences; and
(6) DNA consisting of a nucleotide sequence of the genome sequence of an industrial yeast other than from open reading frames which consists of a nucleotide sequence having identity of more than 94% to the nucleotide sequence of the gene of Saccharomyces cerevisiae, or a nucleotide sequence complementary to the above nucleotide sequence, or a nucleotide sequence of continuous 10 or more nucleotides selected from the above nucleotide sequences; and
IV) DNAs of the following (5′) and/or (6′):
(5′) DNA consisting of a nucleotide sequence which contains mismatch of 1 or more base(s) to the DNA of (5), or a nucleotide sequence complementary to the above nucleotide sequence; and
(6′) DNA consisting of a nucleotide sequence which contains mismatch of 1 or more base(s) to the DNA of (6), r a nucleotide sequence complementary to the above nucleotide sequence.

20. The DNA array according to claim 12 or 19, wherein the DNAs are oligonucleotides consisting of 10-30 nucleotides.

21. A method for analyzing genes of an industrial yeast comprising hybridizing genes of the industrial yeast to the DNA array according to claim 12 or 19.

22. A method for classifying an industrial yeast comprising
(a) hybridizing genomic DNA prepared from the industrial yeast to the DNA array of claim 15 or 19; and
(b) calculating percentage to hybridize DNAs of any of (1) to (4), and percentage to hybridize DNAs of (5) and/or (6).

23. The method for classification according to claim 22,
wherein the DNA array comprises at least one group of DNAs selected from (1) to (4), at least one group of DNAs selected from (7) to (10), DNAs of (5) and/or (6) and DNAs of (5′) and/or (6′), and
percentage of genes which hybridize to DNAs of (1) and/or (2), but not to DNAs of (7) and/or (8) is determined as percentage to hybridize DNAs of any of (1) to (4), and percentage of genes which hybridize to DNAs of (5) and/or (6), but not to DNAs of (5′) and/or (6′) is determined as percentage to hybridize DNAs of any of (5) and/or (6).

24. A method for detecting nucleotide polymorphism in genome of an industrial yeast comprising
(a) hybridizing genomic DNA prepared from the industrial yeast to the DNA array of any one of claims 17 or 19; and
(b) selecting a gene wherein hybridization intensity to the DNAs of any of (7) to (10) is higher than hybridization intensity to the DNAs of any of (1) to (4).

25. A method for screening a useful strain of industrial yeast, comprising
(a) hybridizing genomic DNA prepared from the industrial yeast strain to the DNA array of claim 12 or 19; and
(b) determining the industrial yeast strain as useful when the strain contain 1 or more genes wherein hybridization intensity thereof is 1.5 times higher, or ⅔ or less compared to an average hybridization intensity to the DNAs of any of (1) to (4).

26. A method for screening a gene of an industrial yeast, comprising
(a) hybridizing genomic DNA prepared from the industrial yeast strain to the DNA array of claim 12 or 19; and
(b) selecting a gene wherein hybridization intensity thereof is 1.5 times higher, or ⅔ or less compared to an average hybridization intensity to the DNAs of any of (1) to (4).

27. A method for screening a gene of an industrial yeast, comprising
(a-1) hybridizing genomic DNA, cDNA or cRNA prepared from the industrial yeast to a DNA array comprising one or more of DNAs, wherein each DNA is selected from at least one group of (1) to (4):
(1) DNA consisting of a nucleotide sequence of an open reading frame of the genome sequence or an industrial yeast which encodes an amino acid sequence having 70 to 97% identity to an amino acid sequence encoded by the gene of Saccharomyces cerevisiae, or a nucleotide sequence complementary to the above nucleotide sequence, or a nucleotide sequence of continuous 10 or more nucleotides selected from the above nucleotide sequences;
(2) DNA consisting of a nucleotide sequence of the genome sequence of an industrial yeast other than from open reading frames which consists of a nucleotide sequence of having 60 to 94% identity to the nucleotide sequence of the gene of Saccharomyces cerevisiae, or a nucleotide sequence complementary to the above nucleotide sequence, or a nucleotide sequence of continuous 10 or more nucleotides selected from the above nucleotide sequences;
(3) DNA having a nucleotide sequence of any of SEQ ID Nos: 166490 to 167042, or DNA having a nucleotide sequence of any of SEQ ID NOs: 167043 to 173124; and
(4) DNA having a nucleotide sequence of any of SEQ ID NOs: 173125 to 174603, or DNA having a nucleotide sequence of any of SEQ ID NOs: 174604 to 190810:
(a-2) independently from the step of (a-1), hybridizing genomic DNA, cDNA or cRNA prepared from the industrial yeast of the strain used for preparing the DNA array to the DNA array comprising one or more of DNAs, wherein each DNA is selected from at least one group of (1) to (4):
(1) DNA consisting of a nucleotide sequence of an open reading frame of the genome sequence of an industrial yeast which encodes an amino acid sequence having 70 to 97% identity to an amino acid sequence encoded by the gene of Saccharomyces cerevisiae, or a nucleotide sequence complementary to the above nucleotide sequence, or a nucleotide sequence of continuous 10 or more nucleotides selected from the above nucleotide sequences;
(2) DNA consisting of a nucleotide sequence of the genome sequence of an industrial yeast other than from pen reading frames which consists of a nucleotide sequence having 60 to 94% identity to the nucleotide sequence of the gene of Saccharomyces cerevisiae, or a nucleotide sequence complementary to the above nucleotide sequences;
(3) DNA having a nucleotide sequence of any of SEQ ID NOs: 166490 to 167042, or DNA having a nucleotide sequence of any of SEQ ID NOs: 167043 to 173124; and
(4) DNA having a nucleotide sequence of any of SEQ ID NOs: 173125 to 174603, or DNA having a nucleotide sequence of any of SEQ ID NOs: 174604 to 190810; and
(b) selecting a gene wherein hybridization intensity thereof to the DNAs of any of (1) to (4) in the step (a-1) is significantly different from hybridization intensity thereof in the step (a-2).

28. A method for screening a gene of an industrial yeast, comprising
(a-1) hybridizing cDNA or cRNA prepared from the industrial yeast to a DNA array comprising one or more of DNAs, wherein each DNA is selected from at least one group of (1) to (4):
(1) DNA consisting of a nucleotide sequence of an open reading frame of the genome sequence of an industrial yeast which encodes an amino acid sequence having 70 to 97% identity to an amino acid sequence encoded by the gene of Saccharomyces cerevisiae, or a nucleotide sequence complementary to the above nucleotide sequence, or a nucleotide sequence of continuous 10 or more nucleotides selected from the above nucleotide sequences;
(2) DNA consisting of a nucleotide sequence of the genome sequence of an industrial yeast other than from open reading frames which consists of a nucleotide sequence having 60 to 94% identity to the nucleotide sequence of the gene of Saccharomyces cerevisiae, or a nucleotide sequence complementary to the above nucleotide sequence, or a nucleotide sequence of continuous 10 or more nucleotides selected from the above nucleotide sequences;
(3) DNA having a nucleotide sequence of any of SEQ ID NOs: 166490 to 167042, or DNA having a nucleotide sequence of any of SEQ ID NOs: 167043 to 173124; and
(4) DNA having a nucleotide sequence of any of SEQ ID NOs: 173125 to 174603, or DNA having a nucleotide sequence of any of SEQ ID NOs: 174604 to 190810;
(a-2) independently from the step of (a-1), hybridizing cDNA or cRNA prepared from another industrial yeast to a DNA array comprising one or more of DNAs, wherein each DNA is selected from at least one group of (1) to (4):
(1) DNA consisting of a nucleotide sequence of an open reading frame of the genome sequence of an industrial yeast which encodes an amino acid sequence having 70 to 97% identity to an amino acid sequence encoded by the gene of Saccharomyces cerevisiae, or a nucleotide sequence complementary to the above nucleotide sequence, or a nucleotide sequence of continuous 10 or more nucleotides selected from the above nucleotide sequences;
(2) DNA consisting of a nucleotide sequence of the genome sequence of an industrial yeast other than from open reading frames which consists of a nucleotide sequence having 60 to 94% identity to the nucleotide sequence of the gene of Saccharomyces cerevisiae, or a nucleotide sequence complementary to the above nucleotide sequence, or a nucleotide sequence of continuous 10 or more nucleotides selected from the above nucleotide sequences;
(3) DNA having a nucleotide sequence of any of SEQ ID NOs: 166490 to 167042, or DNA having a nucleotide sequence of any of SEQ ID NOs: 167043 to 173124; and
(4) DNA having a nucleotide sequence of any of SEQ ID NOs: 173125 to 174603, or DNA having a nucleotide sequence of any of SEQ ID NOs: 174604 to 190810;
wherein the industrial yeast of (a-2) has been cultured in a different condition from the culture condition for the industrial yeast of (a-1); and
(b) selecting a gene wherein hybridization intensity thereof to the DNAs of any of (1) to (4) in the step (a-1) is significantly different from hybridization intensity thereof in the step (a-2).

29. A gene obtained by the screening method of claim 26.

30. The gene according to claim 9, characterized in that concentration of sulfite in a culture medium of an industrial yeast increases when the gene is expressed in the yeast.

31. A nucleic acid encoding a polypeptide of any one of the following i) and ii):
i) a polypeptide having the amino acid sequence represented by SEQ ID NO:3, and
ii) a polypeptide having an amino acid sequence wherein one or more amino acid residue(s) is deleted, substituted and/or added to the amino acid sequence represented by SEQ ID NO:3, and having an activity to increase concentration of sulfite in a culture medium of an industrial yeast when the gene is expressed in the yeast.

32. The nucleic acid according to claim 31, which is selected from the following a) and b):
(a) a nucleic acid having the nucleotide sequence represented by SEQ ID NO:1; and
(b) a nucleic acid having a nucleotide sequence which hybridizes to the nucleotide sequence complementary to the nucleotide sequence represented by SEQ ID NO:1 under a stringent condition, and encodes a polypeptide having an activity to increase concentration of sulfite in a culture medium of an industrial yeast when the gene is expressed in the yeast.

33. A recombinant vector containing the gene of claim 9.

34. A transformant comprising the recombinant vector of claim 33.

35. The transformant according to claim 34, which is yeast of genus Saccharomyces.

36. A polypeptide of any one of the following i) and ii):
i) a polypeptide having the amino acid sequence represented by SEQ ID NO:3; and
ii) a polypaptide having an amino acid sequence wherein one or more amino acid residue(s) is deleted, substituted and/or added to the amino acid sequence represented by SEQ ID N:3, and having an activity to increase concentration of sulfite in a culture medium of an industrial yeast when the gene is expressed in the yeast.

37. The polypeptide according to claim 36, which is encoded by a nucleic acid selected from the following a) and b):
(a) a nucleic acid having the nucleotide sequence represented by SEQ ID NO:1; and
(b) a nucleic acid having a nucleotide sequence which hybridizes to the nucleotide sequence complementary to the nucleotide sequence represented by SEQ ID NO:1 under a stringent condition, and encodes a polypeptide having an activity to increase concentration of sulfite in a culture medium of an industrial yeast when the gene is expressed in the yeast.

38. A method for producing an alcohol or an alcoholic beverage, comprising culturing the transformant of claim 34.

39. A breeding method of yeast which is suitable for the production of an alcohol or an alcoholic beverage, characterized in that, expression of the gene of claim 9 is controlled.

40. The breeding method according to claim 39, wherein the yeast belongs to the genus Saccharomyces.

41. Yeast obtained by the breeding method of claim 39.

42. A method for producing an alcohol or an alcoholic beverage comprising culturing the yeast of claim 41.

43. An alcohol or an alcoholic beverage which is produced by the producing method of claim 42.

44. The gene according to claim 29 characterized in that concentration of sulfite in a culture medium of an industrial yeast increases when the gene is expressed in the yeast.

45. A recombinant vector containing the gene of claim 29.

46. A recombinant vector containing the nucleic acid of claim 21.

47. A breeding method of yeast which is suitable for the production of an alcohol or alcoholic beverage, characterized in that, expression of the gene of claim 29 is controlled.

48. A breeding method of yeast which is suitable for the production of an alcohol or an alcoholic beverage, characterized in that, expression of the nuclec acid of claim 31 is controlled.
